(19) United States
(12) Patent Application Publication
(10) Pub. No.: US 2024/0239752 A1
(43) Pub. Date: Jul. 18, 2024

(54) BICYCLIC HETEROAROMATIC INHIBITORS OF KLK5

(71) Applicant: BioCryst Pharmaceuticals, Inc., Durham, NC (US)

(72) Inventors: Pravin L. Kotian, Hoover, AL (US); Yarlagadda S. Babu, Birmingham, AL (US); Weihe Zhang, Vestavia Hills, AL (US); Peng-Cheng Lu, Vestavia Hills, AL (US); Zhao Dang, Vestavia Hills, AL (US); Krishnan Raman, Birmingham, AL (US)

(21) Appl. No.: 18/286,615

(22) PCT Filed: Apr. 14, 2022
(86) PCT No.: PCT/US22/24805

§ 371 Date: Oct. 12, 2023

Related U.S. Application Data
(60) Provisional application No. 63/174,860, filed on Apr. 14, 2021.

(51) Int. Cl. C07D 231/56; A61K 31/343; A61K 31/352; A61K 31/416; A61K 31/4178; A61K 31/4184; A61K 31/4196; A61K 31/423; A61K 31/427; A61K 31/437; A61K 31/4375; A61K 31/443; A61K 31/4439; A61K 31/47; A61K 31/4709; A61K 31/4725; A61K 31/496; A61K 31/5025; A61K 31/517; A61K 31/519; C07D 217/22; C07D 307/81; C07D 311/96; C07D 401/04; C07D 401/14; C07D 403/04; C07D 403/10; C07D 405/04; C07D 405/10; C07D 405/12; C07D 405/14; C07D 413/04; C07D 417/12; C07D 471/04; C07D 487/04

(52) U.S. Cl. CPC C07D 231/56; A61K 31/343; A61K 31/352; A61K 31/416; A61K 31/4178; A61K 31/4184; A61K 31/4196; A61K 31/423; A61K 31/427; A61K 31/437; A61K 31/4375; A61K 31/443; A61K 31/4439; A61K 31/47; A61K 31/4709; A61K 31/4725; A61K 31/496; A61K 31/5025; A61K 31/517; A61K 31/519; C07D 217/22; C07D 307/81; C07D 311/96; C07D 401/04; C07D 401/14; C07D 403/04; C07D 403/10; C07D 405/04; C07D 405/10; C07D 405/12; C07D 405/14; C07D 413/04; C07D 417/12; C07D 471/04; C07D 487/04

(57) ABSTRACT

Disclosed are compounds of formulae (I)-(IV), and pharmaceutically acceptable salts thereof, which are inhibitors of kallikrein-related peptidase 5 (KLK5). Also provided are pharmaceutical compositions comprising such a compound, and methods of using the compounds and compositions in the treatment or prevention of a disease or condition characterized by aberrant KLK5 activity, such as Netherton Syndrome.

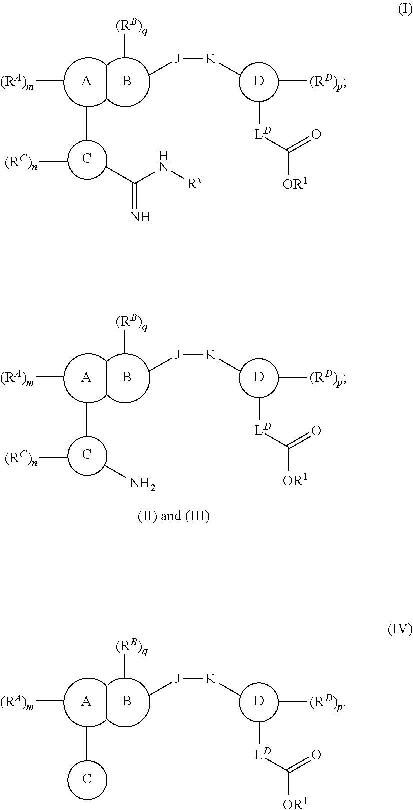

### RELATED APPLICATIONS

This application claims the benefit of priority to U.S. Provisional Patent Application Ser. No. 63/174,860, filed Apr. 14, 2021.

### BACKGROUND OF THE INVENTION

Tissue kallikreins (KLKs) are a family of 15 trypsin- and chymotrypsin-like serine proteases serine proteases. KLKs are secreted as pro-enzymes, requiring the removal of terminal peptide portions through specific amino-terminal proteolysis for activation. Some KLKs are reliant on activation by other KLKs or other proteases, while some KLKs, such as KLK5, are capable of self-activation. As such, KLKs function through proteolytic cascades in the body. KLKs are widely expressed in a diverse range of tissues including the kidney, brain, and respiratory, gastrointestinal, epidermis, and reproductive tracts. KLKs regulate a number of essential physiological functions generally in a cell-specific manner, including modulating immunity and carcinogenesis.

In addition to other roles. KLKs play an important role in the epidermis, such as maintaining the integrity of the skin barrier, and in preventing skin peeling (desquamation) and inflammation. In addition to KLK5, KLK7, and KLK14, 5 additional KLKs are expressed in the skin. KLK5 is considered to be the most important KLK family member and is responsible for initiating the KLK activation cascades. KLK5 hyperactivity leads to the degradation of components of the adhesion complexes which attach the last living layer of the epidermis to the stratum corneum, resulting in cleavage of these structures and premature detachment of the stratum corneum. KLK5 hyperactivity also activates protease activated receptor-2 (PAR-2) resulting in the production of pro-inflammatory cytokines leading to the generation of a pro-inflammatory environment, activation of Langerhans cells, and induction of pro-allergic Th2 cells.

While KLKs play an important role in normal physiology, dysregulation of KLK expression and/or activity can damage healthy cells and tissues. The inappropriate activation of KLKs, including KLK5, is implicated in number of human diseases and conditions (Paliouras, M, Biol Chem. 2006, Vol 387, pages 643-652). Accordingly, there exists a need to develop further KLK inhibitors, which have therapeutic potential in the treatment of numerous disorders.

### SUMMARY OF THE INVENTION

In certain aspects, the invention provides compounds having the structure of formula (I), and pharmaceutically acceptable salts thereof:

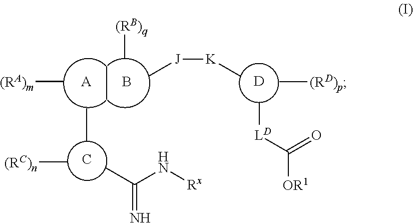

wherein:
ring

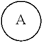

is arylene or heteroarylene;
ring

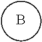

is arylene or heteroarylene;
ring

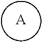

is fused to ring

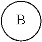

at two and only two adjacent positions;
ring

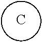

is aryl or heteroaryl;
ring

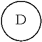

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or

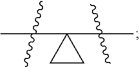 [SMILES: *C1(*)CC1]

K represents a bond, —O—, —NH—, —C(O)—, —CH₂—, —S—, —S(O)—, —SO₂—, —N(alkyl)-, —CH(alkyl)-, or —CH(cycloalkyl)-;
   wherein at least one of J and K is a bond, —C(O)—, —CH₂-, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl), —C(O)O(alkyl), —C(O)NH(optionally substituted alkyl), C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{x} is H or OH;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl. (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

In certain aspects, the invention provides compounds having the structure of formula (II), and pharmaceutically acceptable salts thereof:

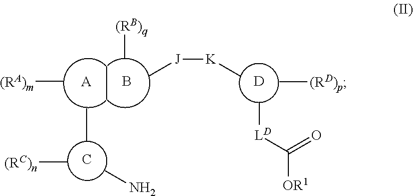

wherein:
ring

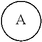

is arylene or heteroarylene;
ring

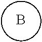

is arylene or heteroarylene;
ring

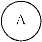

is fused to ring

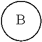

at two and only two adjacent positions;
ring

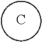

is a bicyclic ring system, where the ring attached to ring

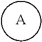

is aryl or heteroaryl;
ring

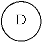

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)-, —CH(cycloalkyl)-, or

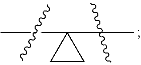

K represents a bond, —O—, —NH—, —C(O)—, —CH₂—, —S—, —S(O)—, —SO₂—, —N(alkyl)-, —CH(alkyl)-, or —CH(cycloalkyl)-;
   wherein at least one of J and K is a bond, —C(O)—, —CH₂—, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—. —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl). —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl). —C(O)O(alkyl). —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl). —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl. (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

In certain aspects, the invention provides compounds having the structure of formula (III), and pharmaceutically acceptable salts thereof:

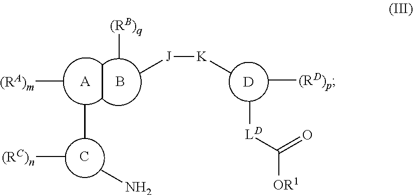

wherein:
ring

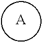

is arylene or heteroarylene;
ring

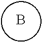

is cycloalkylene, heterocycloalkylene, cycloalkenylene, or heterocycloalkenylene;
ring

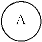

is fused to ring

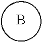

at two and only two adjacent positions;
ring

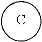

is a bicyclic ring system, where the ring attached to ring

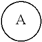

is aryl or heteroaryl;
ring

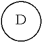

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or

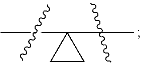

K represents a bond, —O—, —NH—, —C(O)—, —CH₂—, —S—, —S(O)—, —SO₂—, —N(alkyl)-, —CH(alkyl)-, or —CH(cycloalkyl)-;
wherein at least one of J and K is a bond, —C(O)—, —CH₂—, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—. —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl). —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl). —C(O)O(alkyl). —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl). —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two adjacent occurrences of R^{B} taken together with the intervening atoms form an aromatic ring; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl. (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

In certain aspects, the invention provides compounds having the structure of formula (IV), and pharmaceutically acceptable salts thereof:

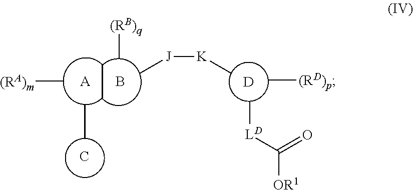

wherein:
ring

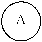

is arylene or heteroarylene;
ring

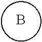

is arylene or heteroarylene;
ring

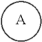

is fused to ring

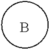

at two and only two adjacent positions;
ring

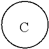

is selected from the group consisting of

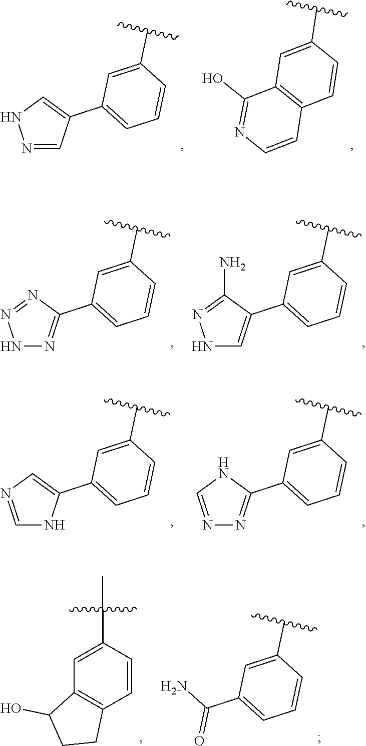

ring

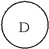

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or

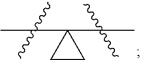 [SMILES: *C1(*)CC1]

K represents a bond, —O—, —NH—, —C(O)—, —CH₂—, —S—, —S(O)—, —SO₂—, —N(alkyl)-, —CH(alkyl)-, or —CH(cycloalkyl)-;
   wherein at least one of J and K is a bond, —C(O)—, —CH₂—, —CH₂CH₂—, —CH(alkyl)-, or —CH(aryl)-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl). —CH₂O(optionally substituted aryl), —C(O)O(alkyl), —C(O)NH(optionally substituted alkyl), —C(O)NH((cycloalkyl)alkyl), —NHC(O)Oalkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, p, and q are each independently 0, 1, or 2.

In certain aspects, the invention provides a pharmaceutical composition, comprising a compound of the invention, or a pharmaceutically acceptable salt thereof; and a pharmaceutically acceptable carrier.

In certain aspects, the invention provides methods of treating a disease or condition characterized by aberrant kallikrein activity, such as aberrant kallikrein-related peptidase 5 (KLK5) activity.

### DETAILED DESCRIPTION

Inhibitors of the kallikrein-related peptidase 5 (KLK5) are useful in therapeutic methods and compositions suitable for use in treating or preventing a disease or condition characterized by aberrant kallikrein activity, e.g., aberrant KLK5 activity. Provided herein are compounds of formulae (I)-(IV) and pharmaceutically acceptable salts thereof that are useful in treating or preventing a disease or condition characterized by aberrant kallikrein activity, e.g., aberrant KLK5 activity. Such a disease or condition includes, but is not limited to, a skin disease, such as, but not limited to, Netherton Syndrome.

### Definitions

The articles “a” and “an” are used herein to refer to one or to more than one (i.e., to at least one) of the grammatical object of the article. By way of example, “an element” means one element or more than one element.

The term “heteroatom” is art-recognized and refers to an atom of any element other than carbon or hydrogen. Illustrative heteroatoms include boron, nitrogen, oxygen, phosphorus, sulfur and selenium, and alternatively oxygen, nitrogen or sulfur.

The term “alkyl” as used herein is a term of art and refers to saturated aliphatic groups, including straight-chain alkyl groups, branched-chain alkyl groups, cycloalkyl groups, alkyl substituted cycloalkyl groups, and (cycloalkyl)alkyl groups. In certain embodiments, a straight-chain or branched-chain alkyl has about 30 or fewer carbon atoms in its backbone (e.g., C₁-C₃₀ for straight chain, C₃-C₃₀ for branched chain), and alternatively, about 20 or fewer, or 10 or fewer. In certain embodiments, the term “alkyl” refers to a C₁-C₁₀ alkyl group. In certain embodiments, the term “alkyl” refers to a C₁-C₆ alkyl group, for example a C₁-C₆ straight-chain alkyl group. In certain embodiments, the term “alkyl” refers to a C₃-C₁₂ branched-chain alkyl group. In certain embodiments, the term “alkyl” refers to a C₃-C₈ branched-chain alkyl group. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “cycloalkyl” means mono- or bicyclic saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇)cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged monocyclic rings and fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocycloalkyl, a monocyclic heterocycloalkenyl, or a monocyclic heteroaryl. The bridged or fused bicyclic cycloalkyl is attached to the parent molecular moiety through any carbon atom contained within the monocyclic cycloalkyl ring. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocycloalkyl, a 5 or 6 membered monocyclic heterocycloalkenyl, or a 5 or 6 membered monocyclic heteroaryl, wherein the fused bicyclic cycloalkyl is optionally substituted.

The term “spirocycloalkyl” as used herein refers to a bicyclic cycloalkyl ring system in which the two rings are linked by a common atom, such as a quaternary carbon atom. The spirocycloalkyl is attached to the parent molecular moiety through any carbon atom contained within the spirocycloalkyl ring system. Suitable spirocycloalkyl groups include, but are not limited to, spiro[2.2]pentane, spiro[3.3]heptane, spiro[4.4.]nonane, spiro[2.3]hexane, and spiro[3.4]octane.

The term “cycloalkylene” as used herein refers to a divalent cycloalkyl group. In some embodiments, a cycloalkylene may be fused to an arylene or heteroarylene group. i.e., a cycloalkylene may be bonded at two adjacent positions to an arylene or heteroarylene group. In such embodiments, the cycloalkylene is saturated at all atoms except the atoms that are fused to the arylene group.

The term “(cycloalkyl)alkyl” as used herein refers to an alkyl group substituted with one or more cycloalkyl groups. An example of cycloalkylalkyl is cyclohexylmethyl group.

The term “cycloalkenyl” as used herein refers to a cycloalkyl group as defined above that additionally contains at least one carbon-carbon double bond. In certain embodiments, the cycloalkenyl is a mono- or bicyclic carbocyclic ring having at least one carbon-carbon double bond and containings from 3 to 12 carbon atoms. For avoidance of doubt, a cycloalkenyl group is not aromatic.

The term “cycloalkynyl” as used herein refers to a cycloalkyl group as defined above that additionally contains at least one carbon-carbon triple bond. In certain embodiments, the cycloalkynyl is a mono- or bicyclic carbocyclic ring having at least one carbon-carbon triple bond and containing from 3 to 12 carbon atoms. For avoidance of doubt, a cycloalkynyl group is not aromatic.

The term “cycloalkenylene” as used herein refers to a divalent cycloalkenyl group. In some embodiments, a cycloalkenylene may be fused to an arylene or heteroarylene group; i.e., a cycloalkenylene may be bonded at two adjacent positions to an arylene or heteroarylene group. In such embodiments, the cycloalkenylene contains at least one saturated carbon atom and at least one carbon-carbon double bond in addition to the atoms that are fused to the arylene group.

The term “heterocycloalkyl” as used herein refers to a radical of a non-aromatic ring system, including, but not limited to, monocyclic, bicyclic, and tricyclic rings, which can be completely saturated or which can contain one or more units of unsaturation, wherein for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiiranyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, ditbictanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, and 2-azobicyclo[3.1.0]hexane. A heterocycloalkyl group may be optionally substituted by one or more substituents as described below.

The term “spiroheterocycloalkyl” as used herein refers to a bicyclic heterocycloalkyl ring system in which the two rings are linked by a common atom, such as a quaternary carbon atom. The spiroheterocycloalkyl is attached to the parent molecular moiety through any carbon atom contained within the spiroheterocycloalkyl ring system.

The term “heterocycloalkylene” as used herein refers to a divalent heterocycloalkyl group. In some embodiments, a heterocycloalkylene may be fused to an arylene or heteroarylene group; i.e., a heterocycloalkylene may be bonded at two adjacent positions to an arylene or heteroarylene group. In such embodiments, the heterocycloalkylene is saturated at all atoms except the atoms that are fused to the arylene group.

The term “(heterocycloalkyl)alkyl” as used herein refers to an alkyl group substituted with one or more heterocycloalkyl (i.e., heterocyclyl) groups.

The term “heterocycloalkenyl” as used herein refers to a heterocycloalkyl group, as defined above, that additionally contains at least one carbon-carbon double bond. For avoidance of doubt, a heterocycloalkenyl group is not aromatic.

The term “heterocycloalkynyl” as used herein refers to a heterocycloalkyl group, as defined above, that additionally contains at least one carbon-carbon triple bond. For avoidance of doubt, a heterocycloalkynyl group is not aromatic.

The term “heterocycloalkenylene” as used herein refers to a divalent heterocycloalkenyl group. In some embodiments, a heterocycloalkenylene may be fused to an arylene or heteroarylene group; i.e., a heterocycloalkenylene may be bonded at two adjacent positions to an arylene or heteroarylene group. In such embodiments, the heterocycloalkenylene contains at least one carbon-carbon double bond in addition to the atoms that are fused to the arylene group.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as those described below. That is, in one embodiment, a “substituted alkyl” is an “alkylene”.

The term “amino” is a term of art and as used herein refers to both unsubstituted and substituted amines, e.g., a moiety that may be represented by the general formulas:

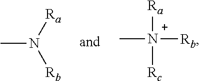

wherein Rₐ, R_{b}, and R_{c} each independently represent a hydrogen, —(CH₂)ₓ-R_{d}, —C(O)-alkyl, —C(O)-alkenyl, where the alkyl or alkenyl may be optionally substituted, or optionally substituted alkyl, alkenyl, alkynyl, aryl, heteroaryl, cycloalkyl, cycloalkenyl, heterocycloalkyl, heterocycloalkenyl, (cycloalkyl)alkyl, (heterocycloalkyl)alkyl, arylalkyl, heteroarylalkyl, alkoxyalkyl, or haloalkyl, or Rₐ and R_{b}, taken together with the N atom to which they are attached form a heterocycle having from 4 to 8 atoms in the ring structure, which may be optionally substituted; R_{d} represents optionally substituted aryl, heteroaryl, cycloalkyl, cycloalkenyl, heterocycloalkyl or polycyclyl; and x is zero or an integer in the range of 1 to 8. In certain embodiments, only one of Rₐ or R_{b} contains a carbonyl adjacent to the N atom, e.g., Rₐ, R_{b}, and the nitrogen together do not form an imide. In other embodiments, Rₐ and R_{b} (and optionally R_{c}) each independently represent hydrogen, optionally substituted alkyl, optionally substituted alkenyl, or —(CH₂)ₓ—R_{d}. In certain embodiments, the term “amino” refers to —NH₂.

In certain embodiments, the term “alkylamino” refers to —NH(alkyl).

In certain embodiments, the term “dialkylamino” refers to —N(alkyl)₂.

The term “amido”, as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “acyl” is a term of art and as used herein refers to any group or radical of the form R^{C}(O)— where R is any organic group, e.g., alkyl, aryl, heteroaryl, arylalkyl, and heteroarylalkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group, i.e., —CH₂NH₂.

The term “aminoacyl” is a term of art and as used herein refers to an acyl group substituted with one or more amino groups.

The term “aminothienyl” is a term of art and as used herein refers to any group or radical of the form RC(S)—, wherein R is any organic group, e.g., alkyl, aryl, heteroaryl, arylalkyl, and heteroarylalkyl.

The term “phosphoryl” is a term of art and as used herein may in general be represented by the formula:

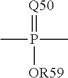

wherein Q50 represents S or O, and R59 represents hydrogen, optionally substituted (C₁-C₆)alkyl or optionally substituted aryl; for example, —P(O)(OMe)- or —P(O)(OH)₂. When used to substitute, e.g., an alkyl, the phosphoryl group of the phosphorylalkyl may be represented by the general formulas:

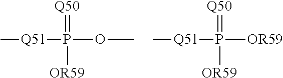

wherein Q50 and R59, each independently, are defined above, and Q51 represents O, S or N; for example, —O—P(O)(OH)OMe or —NH—P(O)(OH)₂. When Q50 is S, the phosphoryl moiety is a “phosphorothioate.”

The term “aminophosphoryl” as used herein refers to a phosphoryl group substituted with at least one amino group, as defined herein; for example, —P(O)(OH)NMe₂.

The term “azide” or “azido”, as used herein, means an —N₃ group.

The term “carbonyl” as used herein refers to —C(═O)—.

The term “thiocarbonyl” as used herein refers to —C(═S)—.

The term “alkylphosphonyl” as used herein refers to a phosphoryl group substituted with at least one alkyl group, as defined herein; for example, —P(O)(OH)Me.

The term “alkylthio” as used herein refers to alkyl-S—. The term “(alkylthio)alkyl” refers to an alkyl group substituted by an alkylthio group.

The term “carboxy”, as used herein, means a —CO₂H group.

The term “aryl” is a term of art and as used herein refers to includes monocyclic, bicyclic and polycyclic aromatic hydrocarbon groups, for example, benzene, naphthalene, anthracene, and pyrene. Typically, an aryl group contains from 6-10 carbon ring atoms (i.e., (C₆-C₁₀)aryl). The aromatic ring may be optionally substituted at one or more ring positions with one or more substituents as described below. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, heterocycloalkyls, heterocycloalkenyls, and/or heterocycloalkynyls. In certain embodiments, the term “aryl” refers to a phenyl group.

The term “arylene” as used herein pertains to a diradical obtained by removing two hydrogen atoms of an aryl group, as defined above. Arylene includes, without limitation, 1,2-phenylene, 1,3-phenylene, and 1,4-phenylene, as depicted below:

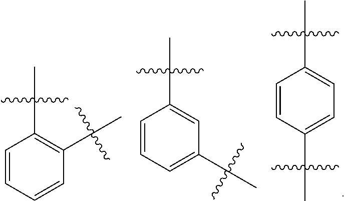 [SMILES: *c1ccccc1* ; *c1cccc(*)c1 ; *c1ccc(*)cc1]

Arylene groups may be optionally substituted at one or more ring positions with one or more substituents, valency permitting, such as the exemplary substituents described below. To provide an exemplary illustration: in certain embodiments of Formula (I), ring

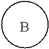

may be arylene, e.g., phenylene. In certain such embodiments, the phenylene is fused to ring

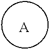

at two and only two adjacent positions, and the ring

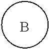

phenylene is additionally bonded to -J- and additionally any present occurrences of R^{B}. The arylene at ring

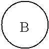

could therefore be represented as follows:

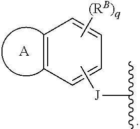

The term “heteroaryl” is a term of art and as used herein refers to a monocyclic, bicyclic, and polycyclic aromatic group having 3 to 12 total atoms including one or more heteroatoms such as nitrogen, oxygen, or sulfur in the ring structure. Exemplary heteroaryl groups include azaindolyl, benzo(b)thienyl, benzimidazolyl, benzofuranyl, benzoxazolyl, benzothiazolyl, benzothiadiazolyl, benzotriazolyl, benzoxadiazolyl, furanyl, 1,3-dihydro-2H-imidazol-2-one, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl, and the like. The “heteroaryl” may be optionally substituted at one or more ring positions with one or more substituents as described below. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, heterocycloalkyls, heterocycloalkenyls, and/or heterocycloalkynyls.

The term “heteroarylene” as used herein pertains to a diradical obtained by removing two hydrogen atoms of a heteroaryl group, as defined above. Heteroarylene includes, without limitation, the divalent heteroarylene groups depicted below:

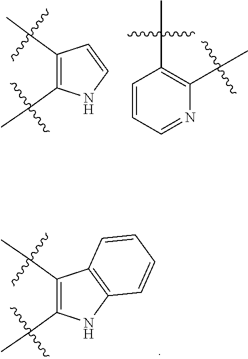

Heteroarylene groups may be optionally substituted at one or more ring positions with one or more substituents, valency permitting, such as the exemplary substituents described below.

The term “aralkyl” or “arylalkyl” is a term of art and as used herein refers to an alkyl group substituted with an aryl group, wherein the moiety is appended to the parent molecule through the alkyl group.

The term “heteroaralkyl” or “heteroarylalkyl” is a term of art and as used herein refers to an alkyl group, as defined herein, substituted with a heteroaryl group, as defined herein, wherein the moiety is appended to the parent molecular moiety through the alkyl group.

The term “alkoxy” as used herein refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “haloalkoxy” as used herein refers to an alkoxy group, as defined herein, wherein some or all of the hydrogens of the alkyl group are replaced with halogen atoms, as defined herein. Representative examples of haloalkoxy include, but are not limited to, —OCF₃.

The term “alkoxyalkyl” as used herein refers to an alkyl group, as defined herein, substituted by an alkoxy group as defined herein.

The term “alkoxycarbonyl” as used herein means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkylcarbonyl”, as used herein, means an alkyl group, as defined herein, appended to the parent molecular moiety through a carbonyl group, represented by —C(═O)—, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “arylcarbonyl”, as used herein, means an aryl group, as defined herein, appended to the parent molecular moiety through a carbonyl group, represented by —C(═O)—, as defined herein. Representative examples of arylcarbonyl include, but are not limited to, benzoyl and (2-pyridinyl)carbonyl.

The term “alkylcarbonyloxy” and “arylcarbonyloxy”, as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkenoxy” or “alkenoxyl” means an alkenyl group, as defined herein, appended to the parent molecular moiety through an oxygen atom. Representative examples of alkenoxyl include, but are not limited to, 2-propen-1-oxyl (i.e., CH₂═CH—CH₂—O—) and vinyloxy (i.e., CH₂═CH—O—).

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl. I-cyclohexenyl and 2-cyclopentenylmethyl.

The term “cyano” is a term of art and as used herein refers to —CN.

The term “halo” is a term of art and as used herein refers to —F, —Cl, —Br, or —I.

The term “haloalkyl” as used herein refers to an alkyl group, as defined herein, wherein some or all of the hydrogens are replaced with halogen atoms, as defined herein. Representative examples of haloalkyl include, but are not limited to, trifluoromethyl and fluoroethyl.

The term “hydroxy” is a term of art and as used herein refers to —OH.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “silyl”, as used herein, includes hydrocarbyl derivatives of the silyl (H₃Si—) group (i.e., (hydrocarbyl)₃Si—), wherein hydrocarbyl groups are univalent groups formed by removing a hydrogen atom from a hydrocarbon. e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy”, as used herein, means a silyl group, as defined herein, is appended to the parent molecule through an oxygen atom.

Certain compounds contained in compositions of the present invention may exist in particular geometric or stereoisomeric forms. In addition, compounds of the present invention may also be optically active. The present invention contemplates all such compounds, including cis- and trans-isomers, (R)- and (S)-enantiomers, diastereoisomers, (D)-isomers. (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

In certain embodiments, the optional substituents contemplated in this invention include halogen, azide, alkyl, alkenyl, alkynyl, aryl, arylalkyl, heteroaryl, heteroarylalkyl, cycloalkyl, cycloalkenyl, cycloalkynyl, (cycloalkyl)alkyl, heterocycloalkyl, heterocycloalkenyl, heterocycloalkynyl, (heterocycloalkyl)alkyl, hydroxyl, alkoxy, amino, aminoalkyl, nitro, sulfhydryl, imino, amido, phosphonate, phosphinate, carbonyl, carboxyl, carboxylalkyl (e.g., -alkylene-(COOH)), silyl, ether (e.g., -alkylene-O(alkyl)), alkylthio, sulfonyl (e.g., —S(O)₂alkyl), sulfonamido, Boc (—C(O)—O—C(CH₃)₃), ketone (e.g., —CO(alkyl)), aldehyde (—C(O)H), ester (e.g., —COO(alkyl)), haloalkyl, hydroxyalkyl, alkoxyalkyl, haloalkoxy, haloalkoxyalkyl, and cyano.

As used herein, the term “optionally substituted” or “substituted or unsubstituted” when it precedes a list of chemical moieties means that the list of chemical moieties that follow are each substituted or unsubstituted. For example, “substituted or unsubstituted aryl, heteroaryl, and cycloalkyl” or “optionally substituted aryl, heteroaryl, and cycloalkyl” means substituted or unsubstituted aryl, substituted or unsubstituted heteroaryl, and substituted or unsubstituted cycloalkyl.

The phrase “protecting group”, as used herein, means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts. P. G. M. Protective Groups in Organic Synthesis, 2^{nd} ed.; Wiley: New York, 1991). Protected forms of the inventive compounds are included within the scope of this invention.

For purposes of the invention, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker. S., 1985), McGraw-Hill, San Francisco, incorporated herein by reference). Unless otherwise defined, all technical and scientific terms used herein have the same meaning as commonly understood by one of ordinary skill in the art to which this invention pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula I. As another example, the salt may comprise less than one inorganic or organic acid molecule per molecule of base, such as two molecules of compound of Formula I per molecule of tartaric acid.

The term “prodrug” as used herein refers to a compound that can be metabolized in vivo to provide a compound of formula I, II, III, or IV. Thus prodrugs include compounds that can be prepared by modifying one or more functional groups in a compound of formula I, II, III, or IV to provide a corresponding compound that can be metabolized in vivo to provide a compound of formula I, II, III, or IV. Such modifications are known in the art. For example, one or more hydroxyl groups or amine groups in a compound of formula I, II, III, or IV can be acylated with alkyl-C(═O), groups or with residues from amino acids to provide a prodrug.

Prodrug forms of a compound bearing various nitrogen-containing functional groups (amino, hydroxyamino, amide, etc.) may include the following types of derivatives, where each Rₚ group individually may be hydrogen, substituted or unsubstituted alkyl, aryl, alkenyl, alkynyl, heterocycle, alkylaryl, arylalkyl, aralkenyl, aralkynyl, cycloalkyl or cycloalkenyl.
(a) Carboxamides, represented as —NHC(O)Rₚ
(b) Carbamates, represented as —NHC(O)ORₚ
(c) (Acyloxy)alkyl Carbamates, represented as NHC(O)OROC(O)Rₚ
(d) Enamines, represented as —NHCR(═CHCO₂Rₚ) or —NHCR(═CHCONRₚRₚ)
(e) Schiff Bases, represented as —N═CRₚRₚ
(f) Mannich Bases (from carboxamide compounds), represented as RCONHCH₂NRₚRₚ

Preparations of such prodrug derivatives are discussed in various literature sources (examples are: Alexander et al, J. Med. Chem. 1988, 31, 318; Aligas-Martin et al., PCT WO0041531, p. 30).

Prodrug forms of carboxyl-bearing compounds include esters (—CO₂Rₘ), where the Rₘ group corresponds to any alcohol whose release in the body through enzymatic or hydrolytic processes would be at pharmaceutically acceptable levels. Another prodrug derived from a carboxylic acid form of the disclosure may be a quaternary salt type of structure described by Bodor et al., J. Med. Chem. 1980, 23, 469

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” as used herein means prevent, halt or slow the progression of, or eliminate a disease or condition in a subject. In one embodiment “treat” means halt or slow the progression of, or eliminate a disease or condition in a subject. In one embodiment, “treat” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments “inhibit” means decrease by at least 5, 10, 20, 30, 40, 50, 60, 70, 80, 90, or 95 percent compared to relevant control. In one embodiment “inhibit” means decrease 100 percent, i.e., halt or eliminate.

The term “subject” as used herein refers to a mammal. In various embodiments, a subject is a mouse, rat, rabbit, cat, dog, pig, sheep, horse, cow, or non-human primate. In one embodiment, a subject is a human.

### Compounds

The present invention provides compounds having the structure of Formula (I), and pharmaceutically acceptable salts thereof:

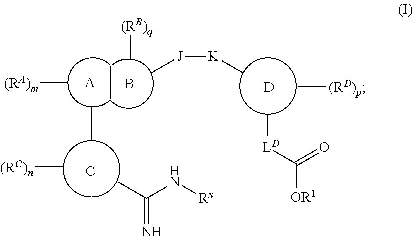

wherein:
ring

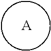

is arylene or heteroarylene;
ring

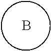

is arylene or heteroarylene;
ring

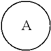

is fused to ring

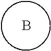

at two and only two adjacent positions;
ring

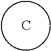

is aryl or heteroaryl;
ring

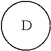

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-. —C(alkyl)₂-, —CH(cycloalkyl)-, or

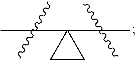

K represents a bond, —O—, —NH—, —C(O)—, —CH₂—, —S—, —S(O)—, —SO₂—, —N(alkyl)-, —CH(alkyl)-, or —CH(cycloalkyl)-;
wherein at least one of J and K is a bond, —C(O)—, —CH₂—, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-. —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl). —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O((optionally substituted aryl), —C(O)O(alkyl), —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH. —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{x} is H or OH;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl. (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

In certain embodiments, in the compound of Formula (I):
ring

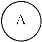

is arylene or heteroarylene;
ring

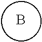

is arylene or heteroarylene;
ring

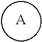

is fused to ring

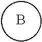

at two and only two adjacent positions;
ring

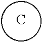

is aryl or heteroaryl;
ring

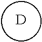

is aryl;
J represents —CH₂— or —C(O)—;
K represents —O—, —NH—, or a bond;
L^{D} represents —CH₂—, —CH(OH)—, or a bond;
R^{A}, independently for each occurrence, represents H, optionally substituted alkoxy, or optionally substituted (heteroaryl)alkoxy;
R^{B}, independently for each occurrence, represents H, oxo, or is optionally substituted alkyl, aryl, cycloalkyl, (cycloalkyl)alkyl, or heterocycloalkyl;
R^{C}, independently for each occurrence, represents H, halo, —OH, or alkoxy;
R^{x} is H or OH;
R^{D}, independently for each occurrence, represents H, or is optionally substituted alkyl or alkoxy;
R¹ represents H; and
m, n, p, and q are each independently 0, 1, or 2.

In certain embodiments, the compound of Formula (I) has the structure of formula (Ia):

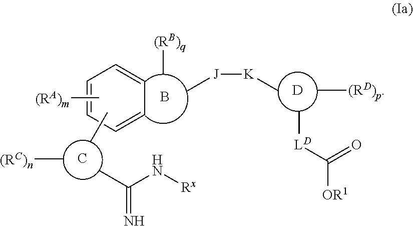

For example, the compound may have the structure of formula (Ia-1):

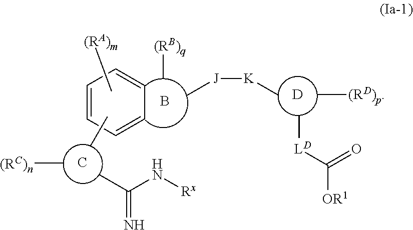

In certain embodiments, the compound of Formula (I) has the structure of formula (Ib)

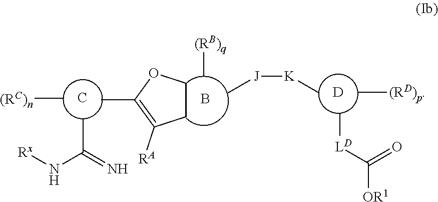

In some embodiments, the compound of Formula (I) has the structure of formula (Ic):

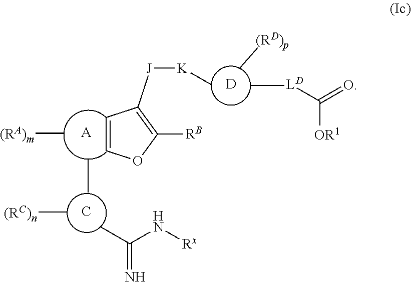

In further embodiments, the compound of Formula (I) has the structure of formula (Id):

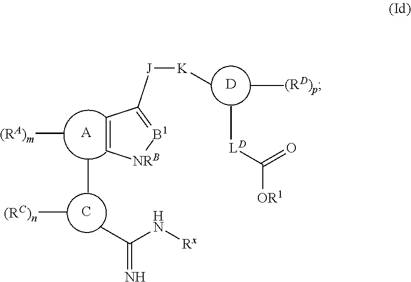

wherein B¹ represents N or CH.

In further embodiments, the compound of Formula (I) has the structure of formula (Ie):

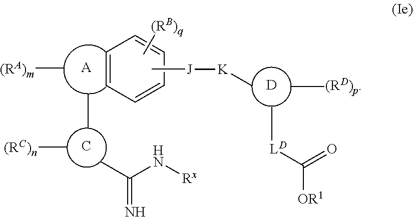

In further embodiments, the compound of Formula (I) has the structure of formula (If):

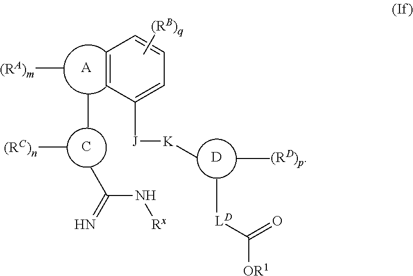

In farther embodiments, the compound of Formula (I) has the structure of any one of formulae (Ig), (Ih), (Ij), or (Ik), depicted below:

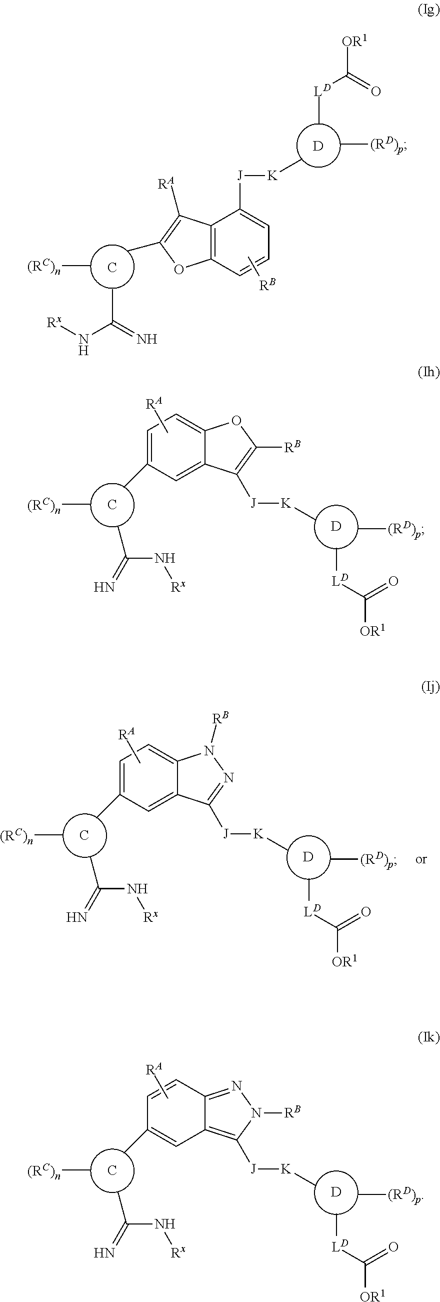

In certain embodiments, the compound of formula (I) is a compound of formula (Ij) or (Ik), wherein:
ring

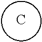

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or
K represents a bond, —O—, —NH—, —C(O)—, —CH₂—, —S—, —S(O)—, —SO₂—, —N(alkyl)-, —CH(alkyl)-, or —CH(cycloalkyl)-;
   wherein at least one of J and K is a bond, —C(O)—, —CH₂—, —CH₂CH₂—, —CH(alkyl)-, or —CH(aryl)₂-;

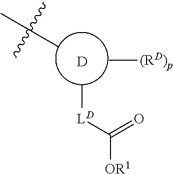

represents

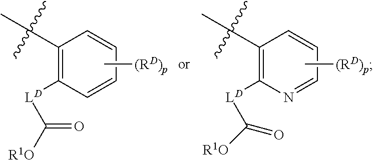

L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R¹ represents H or optionally substituted alkyl;
R^{x} is H or OH
R^{A} is H;
R^{B} is optionally substituted aryl, cycloalkyl, (cycloalkyl)alkyl, heterocycloalkyl, or alkyl;
R^{C} is alkoxy, hydroxy, or halogen;
R^{D} is H, alkoxy or optionally substituted alkyl; and
n and p are each independently 0 or 1.

In certain embodiments, the compound of formula (I) is a compound of the formula (Ij) or (Ik), wherein R^{A} is H, n is 1, and p is 0. In certain embodiments, the compound of formula (I) is a compound of the formula (Ij) or (Ik), wherein R^{A} is H, n is 1, and p is 0, R^{C} is hydroxy, halogen, or an optionally substituted C₁-C₆ alkoxy, and R^{B} is optionally substituted C₁-C₆ alkyl, C₃-C₆ cycloalkyl, C₃-C₆ heterocycloalkyl, C₃-C₆ (cycloalkyl)alkyl.

In certain embodiments, the compound of formula (I) is a compound of the formula (Ij) or (Ik), wherein R^{A} is H, n is 0, and p is 1. In certain embodiments, the compound of formula (I) is a compound of the formula (Ij) or (Ik), wherein R^{A} is H, n is 0, and p is 1, R^{D} is optionally substituted C₁-C₆ alkyl or C₁-C₆ alkoxy, and R^{B} is optionally substituted C₁-C₆ alkyl, C₃-C₆ cycloalkyl, C₃-C₆ heterocycloalkyl. C₃-C₆ (cycloalkyl)alkyl.

In any of the foregoing embodiments of formula (Ij) or (Ik): J is —CH₂— and K is —O—; L^{D} is —CH₂—; R¹ is H; and R^{x} is H;

In any of the foregoing embodiments, the compound of formula (I) is a compound of formula (Ij).

In any of the foregoing embodiments, ring

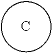

may represent a 6-membered aryl or heteroaryl.

For example, in certain embodiments,

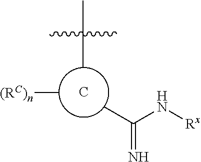

represents

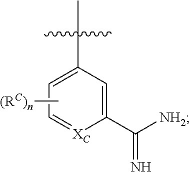

and X_{C} represents CH or N. In further embodiments,

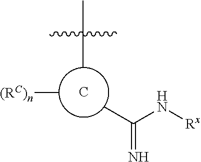

represents

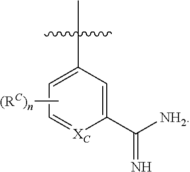

In certain embodiments. R^{C} represents H. Alternatively, R^{C} may represent alkoxy (e.g., methoxy).

In certain embodiments, J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

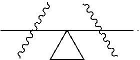 [SMILES: *C1(*)CC1]

In certain embodiments, -J-K- represents —CH₂—O— or —C(O)—NH—.

In any of the foregoing embodiments, ring

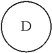

may represent aryl.

For example, in certain embodiments,

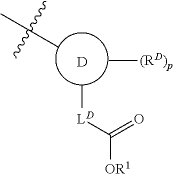

represents

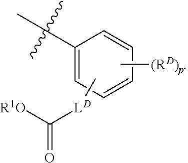

More specifically, in some embodiments,

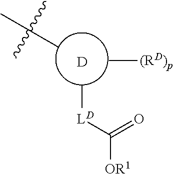

represents

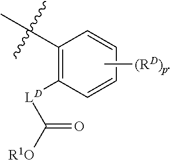

In certain embodiments, ring

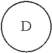

is bicyclic heteroaryl. For example, in some embodiments,

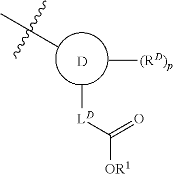

represents

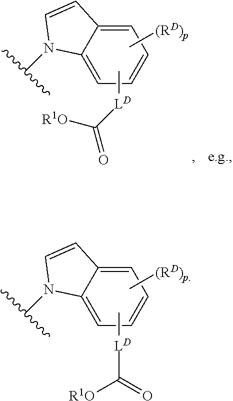

In certain embodiments, m, n, p, and q are each independently 0 or 1.

In certain embodiments, R¹ is H.

In certain embodiments, LP represents —CH₂— or a bond. Preferably, L^{D} represents —CH₂—.

In certain embodiments, R^{D}, independently for each occurrence, represents H, alkyl, or alkoxy. Preferably, R^{D} represents H.

In certain embodiments, R^{B}, independently for each occurrence, represents H, alkyl, cycloalkyl, (cycloalkyl)alkyl, or heterocycloalkyl. In certain embodiments, two geminal occurrences of RE taken together with the atom to which they are attached form an optionally substituted spirocycloalkyl or a spiroheterocycloalkyl. In other embodiments, R^{B} may represent H. Alternatively, R^{B}, independently for each occurrence, may represent alkyl or cycloalkyl.

In certain embodiments, R^{A}, independently for each occurrence, represents H, optionally substituted alkoxy, or optionally substituted (heteroaryl)alkoxy. In some embodiments, R^{A} represents H.

In certain embodiments, the compound of formula (I) is selected from the following table of compounds, and pharmaceutically acceptable salts thereof:

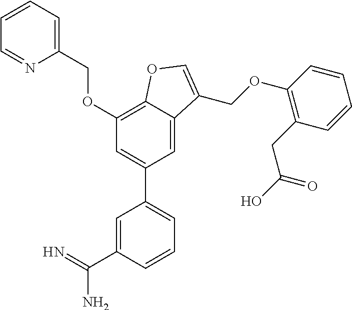 [SMILES: N=C(N)c1cccc(-c2cc(OCc3ccccn3)c3occ(COc4ccccc4CC(=O)O)c3c2)c1]

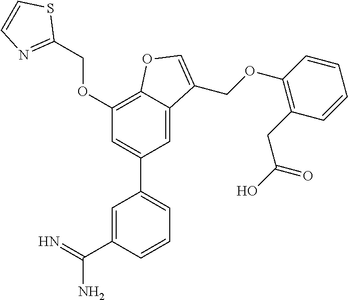 [SMILES: N=C(N)c1cccc(-c2cc(OCc3nccs3)c3occ(COc4ccccc4CC(=O)O)c3c2)c1]

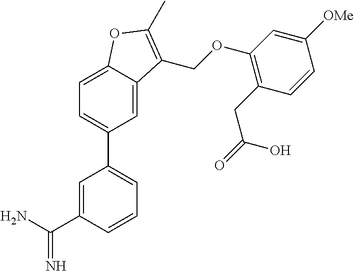 [SMILES: COc1ccc(CC(=O)O)c(OCc2c(C)oc3ccc(-c4cccc(C(=N)N)c4)cc23)c1]

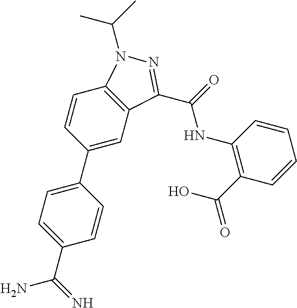 [SMILES: CC(C)n1nc(C(=O)Nc2ccccc2C(=O)O)c2cc(-c3ccc(C(=N)N)cc3)ccc21]

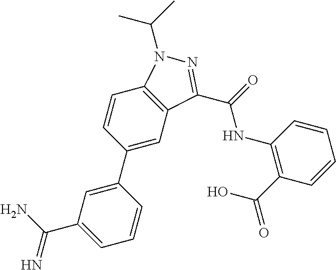 [SMILES: CC(C)n1nc(C(=O)Nc2ccccc2C(=O)O)c2cc(-c3cccc(C(=N)N)c3)ccc21]

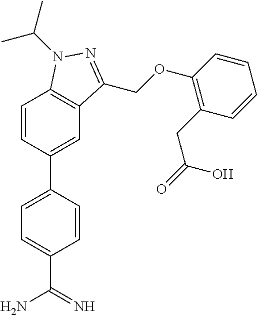 [SMILES: CC(C)n1nc(COc2ccccc2CC(=O)O)c2cc(-c3ccc(C(=N)N)cc3)ccc21]

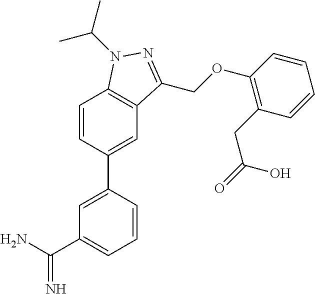 [SMILES: CC(C)n1nc(COc2ccccc2CC(=O)O)c2cc(-c3cccc(C(=N)N)c3)ccc21]

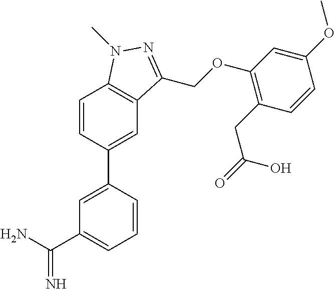 [SMILES: COc1ccc(CC(=O)O)c(OCc2nn(C)c3ccc(-c4cccc(C(=N)N)c4)cc23)c1]

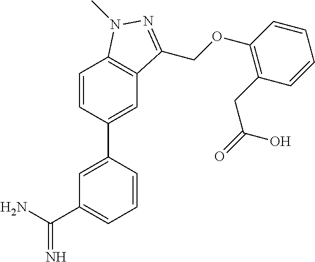 [SMILES: Cn1nc(COc2ccccc2CC(=O)O)c2cc(-c3cccc(C(=N)N)c3)ccc21]

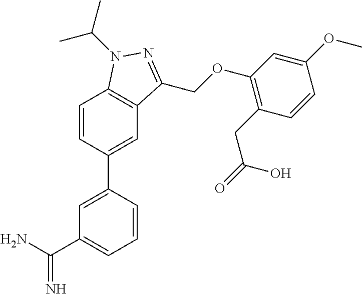 [SMILES: COc1ccc(CC(=O)O)c(OCc2nn(C(C)C)c3ccc(-c4cccc(C(=N)N)c4)cc23)c1]

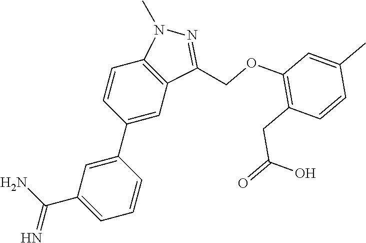 [SMILES: Cc1ccc(CC(=O)O)c(OCc2nn(C)c3ccc(-c4cccc(C(=N)N)c4)cc23)c1]

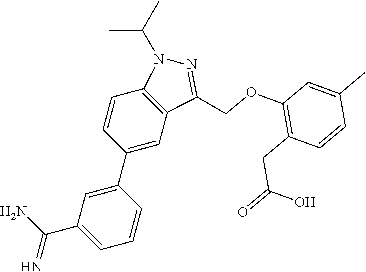 [SMILES: Cc1ccc(CC(=O)O)c(OCc2nn(C(C)C)c3ccc(-c4cccc(C(=N)N)c4)cc23)c1]

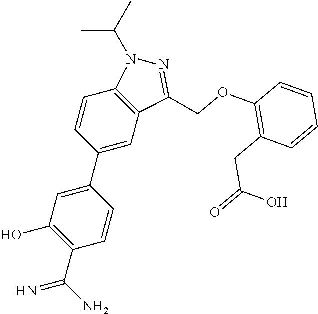 [SMILES: CC(C)n1nc(COc2ccccc2CC(=O)O)c2cc(-c3ccc(C(=N)N)c(O)c3)ccc21]

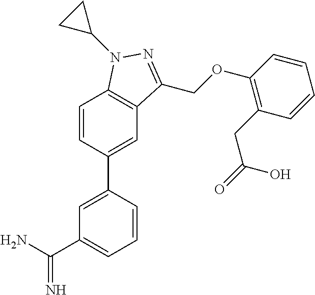 [SMILES: N=C(N)c1cccc(-c2ccc3c(c2)c(COc2ccccc2CC(=O)O)nn3C2CC2)c1]

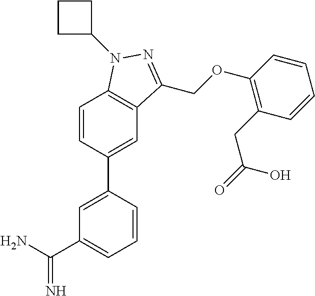 [SMILES: N=C(N)c1cccc(-c2ccc3c(c2)c(COc2ccccc2CC(=O)O)nn3C2CCC2)c1]

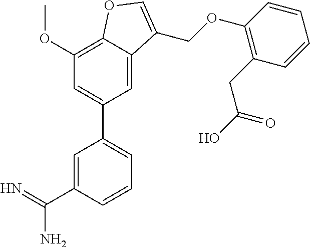 [SMILES: COc1cc(-c2cccc(C(=N)N)c2)cc2c(COc3ccccc3CC(=O)O)coc12]

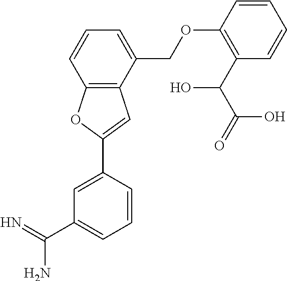 [SMILES: N=C(N)c1cccc(-c2cc3c(COc4ccccc4C(O)C(=O)O)cccc3o2)c1]

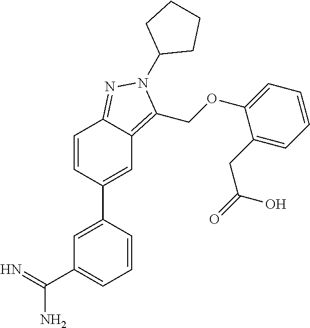 [SMILES: N=C(N)c1cccc(-c2ccc3nn(C4CCCC4)c(COc4ccccc4CC(=O)O)c3c2)c1]

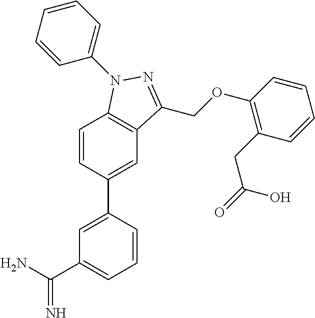 [SMILES: N=C(N)c1cccc(-c2ccc3c(c2)c(COc2ccccc2CC(=O)O)nn3-c2ccccc2)c1]

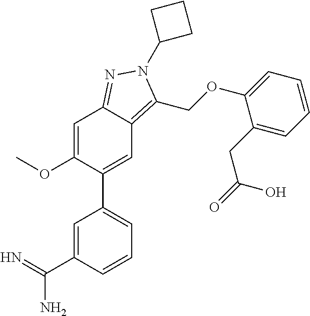 [SMILES: COc1cc2nn(C3CCC3)c(COc3ccccc3CC(=O)O)c2cc1-c1cccc(C(=N)N)c1]

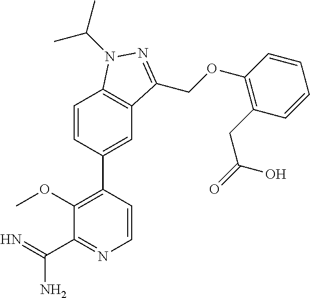 [SMILES: COc1c(-c2ccc3c(c2)c(COc2ccccc2CC(=O)O)nn3C(C)C)ccnc1C(=N)N]

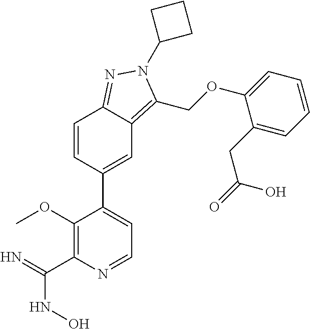 [SMILES: COc1c(-c2ccc3nn(C4CCC4)c(COc4ccccc4CC(=O)O)c3c2)ccnc1C(=N)NO]

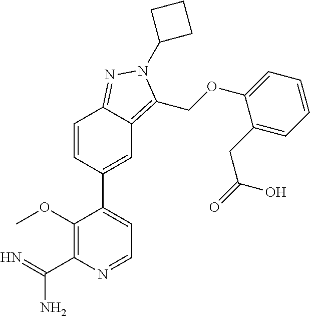 [SMILES: COc1c(-c2ccc3nn(C4CCC4)c(COc4ccccc4CC(=O)O)c3c2)ccnc1C(=N)N]

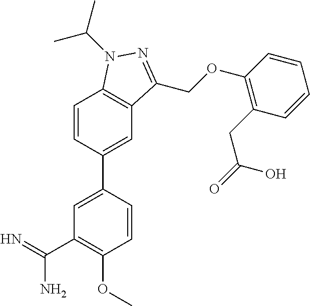 [SMILES: COc1ccc(-c2ccc3c(c2)c(COc2ccccc2CC(=O)O)nn3C(C)C)cc1C(=N)N]

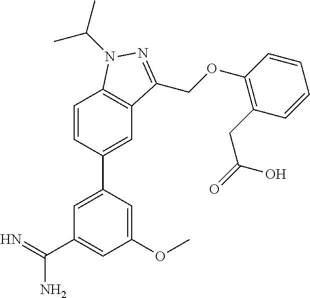 [SMILES: COc1cc(C(=N)N)cc(-c2ccc3c(c2)c(COc2ccccc2CC(=O)O)nn3C(C)C)c1]

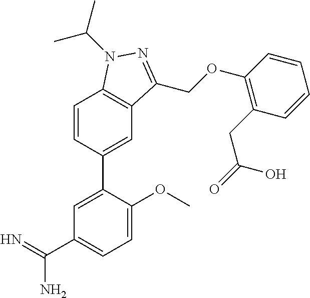 [SMILES: COc1ccc(C(=N)N)cc1-c1ccc2c(c1)c(COc1ccccc1CC(=O)O)nn2C(C)C]

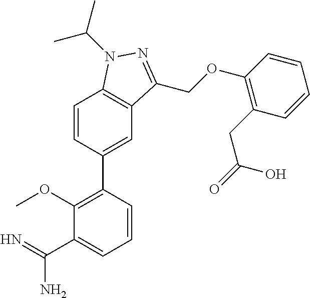 [SMILES: COc1c(C(=N)N)cccc1-c1ccc2c(c1)c(COc1ccccc1CC(=O)O)nn2C(C)C]

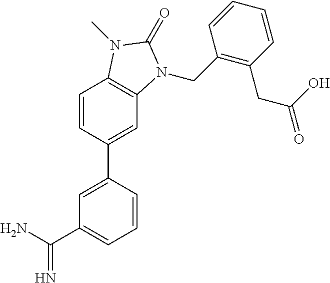 [SMILES: Cn1c(=O)n(Cc2ccccc2CC(=O)O)c2cc(-c3cccc(C(=N)N)c3)ccc21]

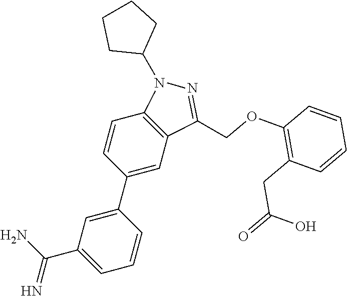 [SMILES: N=C(N)c1cccc(-c2ccc3c(c2)c(COc2ccccc2CC(=O)O)nn3C2CCCC2)c1]

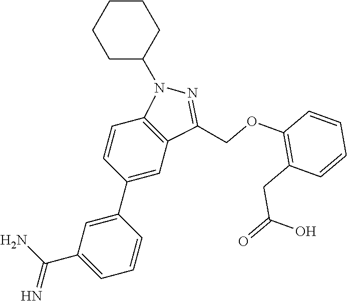 [SMILES: N=C(N)c1cccc(-c2ccc3c(c2)c(COc2ccccc2CC(=O)O)nn3C2CCCCC2)c1]

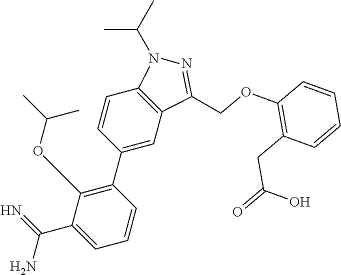 [SMILES: CC(C)Oc1c(C(=N)N)cccc1-c1ccc2c(c1)c(COc1ccccc1CC(=O)O)nn2C(C)C]

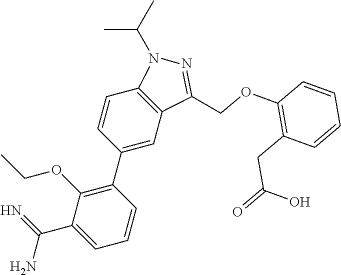 [SMILES: CCOc1c(C(=N)N)cccc1-c1ccc2c(c1)c(COc1ccccc1CC(=O)O)nn2C(C)C]

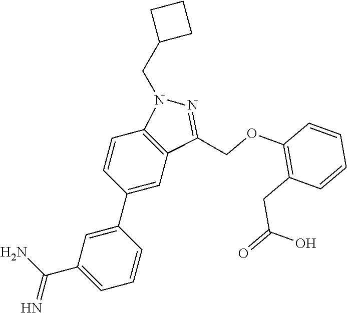 [SMILES: N=C(N)c1cccc(-c2ccc3c(c2)c(COc2ccccc2CC(=O)O)nn3CC2CCC2)c1]

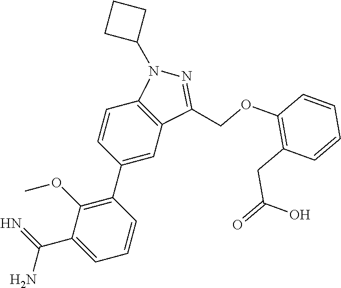 [SMILES: COc1c(C(=N)N)cccc1-c1ccc2c(c1)c(COc1ccccc1CC(=O)O)nn2C1CCC1]

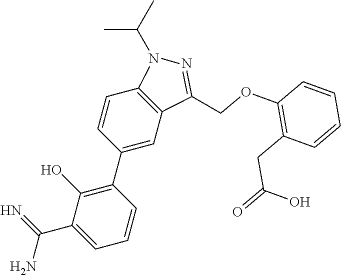 [SMILES: CC(C)n1nc(COc2ccccc2CC(=O)O)c2cc(-c3cccc(C(=N)N)c3O)ccc21]

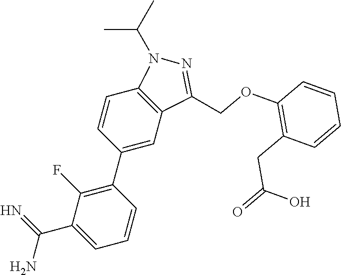 [SMILES: CC(C)n1nc(COc2ccccc2CC(=O)O)c2cc(-c3cccc(C(=N)N)c3F)ccc21]

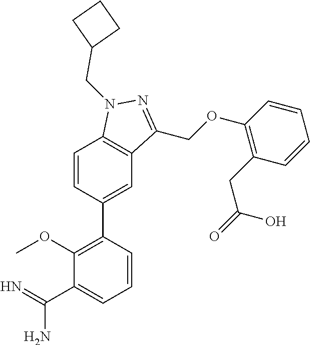 [SMILES: COc1c(C(=N)N)cccc1-c1ccc2c(c1)c(COc1ccccc1CC(=O)O)nn2CC1CCC1]

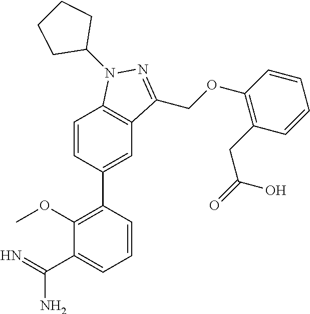 [SMILES: COc1c(C(=N)N)cccc1-c1ccc2c(c1)c(COc1ccccc1CC(=O)O)nn2C1CCCC1]

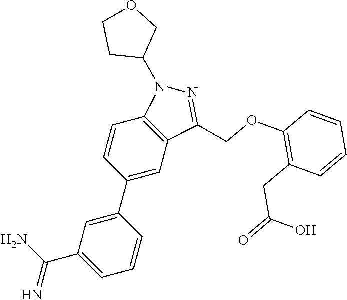 [SMILES: N=C(N)c1cccc(-c2ccc3c(c2)c(COc2ccccc2CC(=O)O)nn3C2CCOC2)c1]

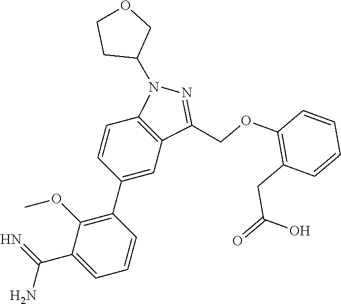 [SMILES: COc1c(C(=N)N)cccc1-c1ccc2c(c1)c(COc1ccccc1CC(=O)O)nn2C1CCOC1]

The present invention provides also compounds having the structure of Formula (II), and pharmaceutically acceptable salts thereof:

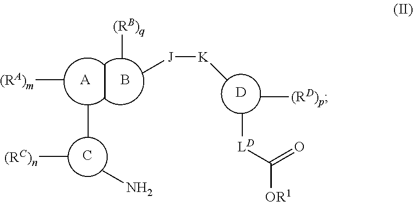

wherein:
ring

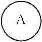

is arylene or heteroarylene;
ring

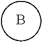

is arylene or heteroarylene;
ring

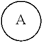

is fused to ring

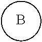

at two and only two adjacent positions;
ring

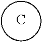

is a bicyclic ring system, where the ring attached to ring

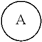

is aryl or heteroaryl;
ring

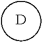

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or

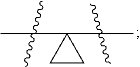 [SMILES: *C1(*)CC1]

K represents a bond, —O—, —NH—, —C(O)—, —CH₂—, —S—, —S(O)—, —SO₂—, —N(alkyl)-, —CH(alkyl)-, or —CH(cycloalkyl)-;
   wherein at least one of J and K is a bond, —C(O)—, —CH₂—, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))-. —CH(NH(cycloalkyl))-, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy. (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl). —C(O)O(alkyl). —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl). —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

In certain embodiments, in the compound of Formula (II):
ring

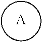

is arylene or heteroarylene;
ring

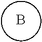

is arylene or heteroarylene;
ring

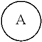

is fused to ring

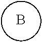

at two and only two adjacent positions;
ring

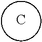

is a bicyclic ring system, where the ring attached to ring

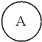

is aryl or heteroaryl;
ring

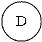

is aryl;
J represents —CH₂— or —C(O)—;
K represents —O— or —NH—;
L^{D} represents —CH₂— or —CH(alkyl)-;
R^{A}, independently for each occurrence, represents H, cyano, alkyl, optionally substituted alkoxy, optionally substituted heteroaryl, optionally substituted aminoalkyl, —C(O)NH₂, or —NH((cycloalkyl)alkyl);
R^{B}, independently for each occurrence, represents H, oxo, alkoxyalkyl, or haloalkyl, or is optionally substituted alkyl, aryl, cycloalkyl, (cycloalkyl)alkyl, spirocycloalkyl, heterocycloalkyl, or (heterocycloalkyl)alkyl;
R^{C}, independently for each occurrence, represents H or halo, or is optionally substituted alkoxy or alkyl;
R^{D}, independently for each occurrence, represents H, halo, cyano, or —C(O)OH, or is optionally substituted alkyl, cycloalkyl, alkoxy, haloalkoxy, or haloalkyl;
R¹ represents H or optionally substituted alkyl.

In certain embodiments, the compound of Formula (II) has the structure of formula (IIa):

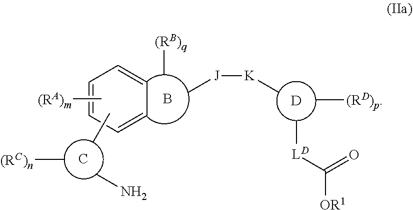

For example, the compound of Formula (II) may have the structure of formula (IIa-1):

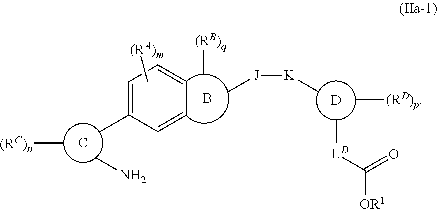

In certain embodiments, the compound of Formula (II) has the structure of formula (IIb):

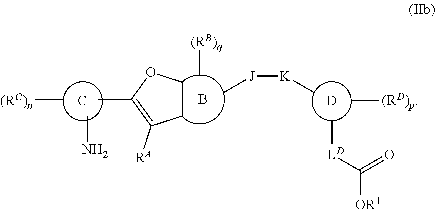

In certain embodiments, the compound of Formula (II) has the structure of formula (IIc):

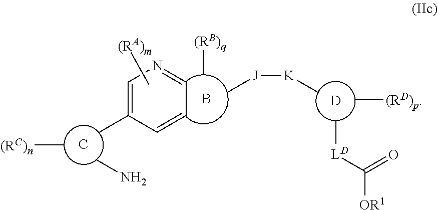

In certain embodiments, the compound of Formula (II) has the structure of formula (IId):

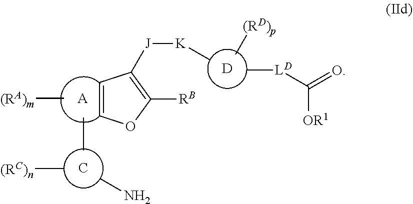

In certain embodiments, the compound of Formula (II) has the structure of formula (Ie):

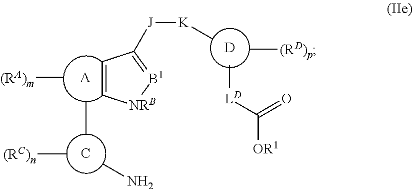

wherein B¹ represents N or CH.

In certain embodiments, the compound of Formula (II) has the structure of formula (IIf):

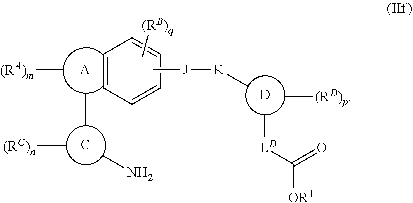

For example, the compound may have the structure of formula (IIf-1):

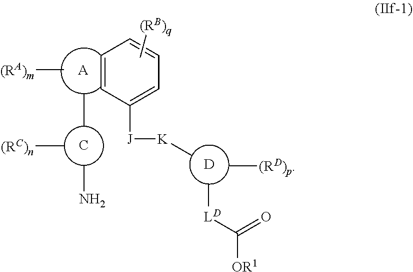

In certain embodiments, the compound of Formula (II) has the structure of formula (IIg), (IIh), (IIj), (IIk), (IIm), (IIn), or (IIo):

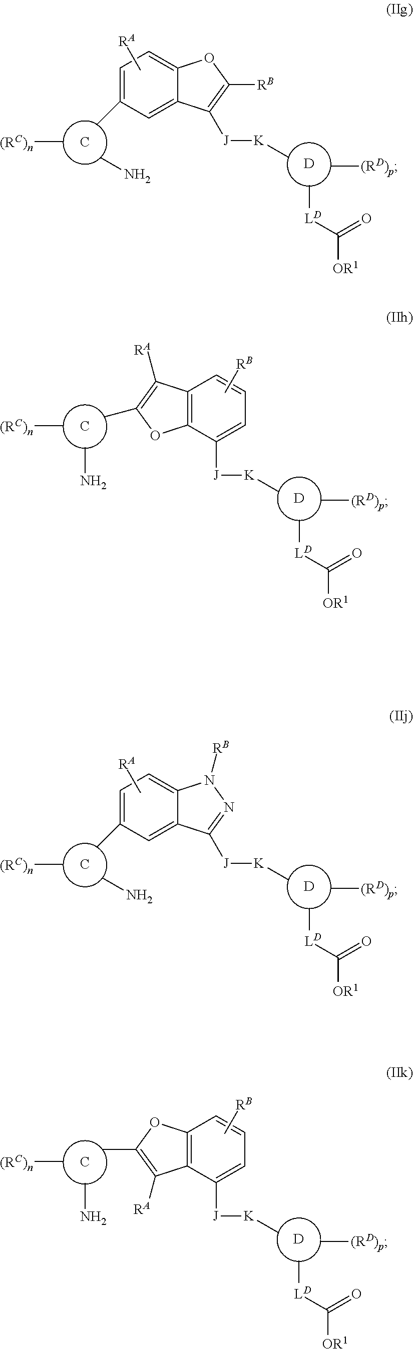

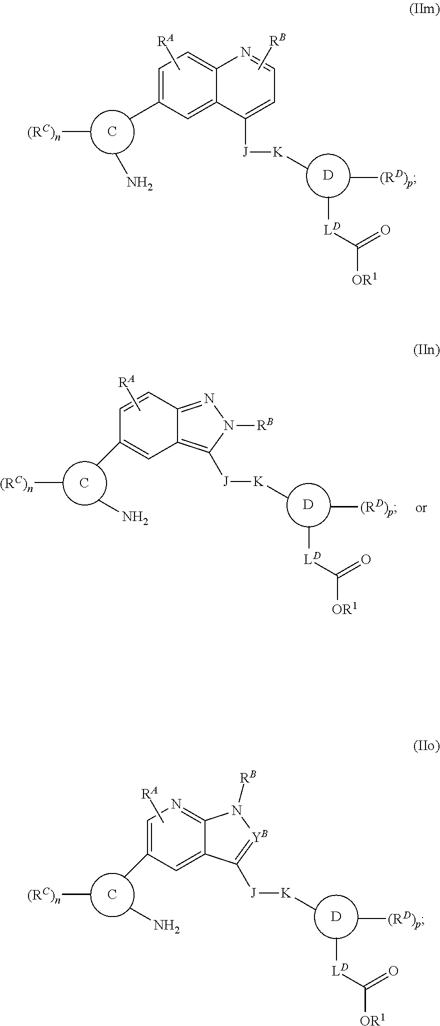

wherein Y^{B} is H or CH.

In some embodiments, ring

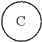

is a bicyclic ring system, where the ring attached to ring

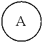

is aryl. Alternatively, ring

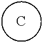

may be a bicyclic ring system, where the ring attached to ring

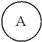

is heteroaryl.

In certain embodiments,

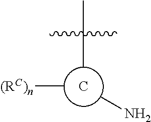

represents

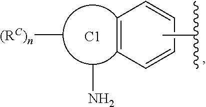

wherein ring

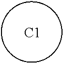

represents aryl, heteroaryl, or heterocycloalkyl. In certain embodiments, ring

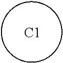

represents heteroaryl, e.g., 5- or 6-membered heteroaryl containing at least one nitrogen atom. In exemplary embodiments,

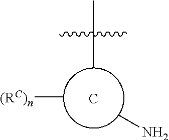

represents

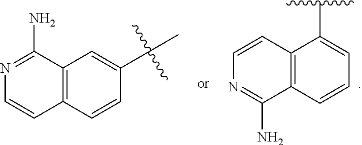 [SMILES: *c1ccc2ccnc(N)c2c1 ; *c1cccc2c(N)nccc12]

Alternatively,

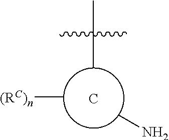

may represent

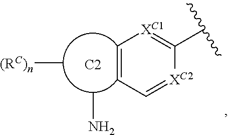

wherein ring

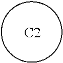

represents aryl, heteroaryl, or heterocycloalkyl; and X^{C1} and X^{C2} each independently represent CH or N. In certain such embodiments, ring

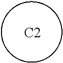

represents aryl or heteroaryl.

In exemplary embodiments,

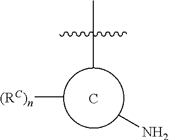

represents

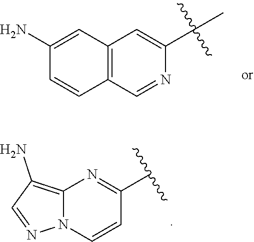 [SMILES: *c1cc2cc(N)ccc2cn1 ; *c1ccn2ncc(N)c2n1]

In certain embodiments, R^{C} represents H. Alternatively, R^{C}, independently for each occurrence, represents alkoxy (e.g., methoxy) or alkyl (e.g., methyl).

In certain embodiments, J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

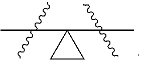 [SMILES: *C1(*)CC1]

In certain embodiments, -J-K- represents —CH₂—O— or —C(O)—NH—.

In any of the foregoing embodiments, ring

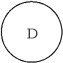

may represent aryl.

For example, in certain embodiments,

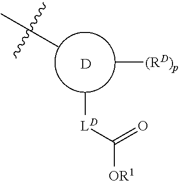

represents

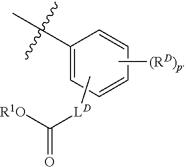

More specifically, in some embodiments,

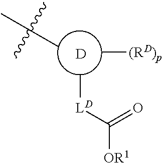

represents

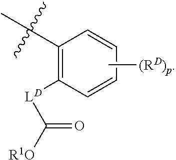

In certain embodiments, ring

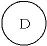

is bicyclic heteroaryl. For example, in some embodiments,

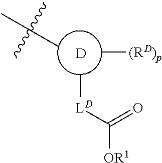

represents

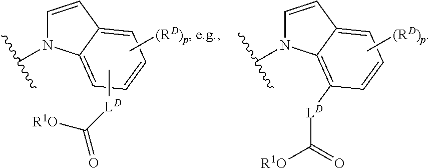

In certain embodiments, m, n, p, and q are each independently 0 or 1.

In certain embodiments, R¹ is H.

In certain embodiments, L^{D} represents —CH₂— or —CH(alkyl)- (e.g., —CH(methyl)-).

Preferably, L^{D} represents —CH₂—.

In certain embodiments, R^{D}, independently for each occurrence, represents H, alkyl, or alkoxy. Preferably, R^{D} represents H.

In certain embodiments, R^{B}, independently for each occurrence, represents H, alkoxyalkyl, or haloalkyl, or is optionally substituted alkyl, aryl, cycloalkyl, (cycloalkyl)alkyl, heterocycloalkyl, or (heterocycloalkyl)alkyl. In certain embodiments, two geminal occurrences of R^{B} taken together with the atom to which they are attached form an optionally substituted spirocycloalkyl or a spiroheterocycloalkyl. In other embodiments. R^{B}, independently for each occurrence, may represent optionally substituted alkyl, cycloalkyl, or heterocycloalkyl, e.g., heterocycloalkyl substituted with alkyl, hydroxyalkyl, alkoxyalkyl. In certain embodiments. R^{B} may represent unsubstituted alkyl.

In certain embodiments, R^{A}, independently for each occurrence, represents H, cyano, alkyl, optionally substituted alkoxy, optionally substituted heteroaryl, or —NH((cycloalkyl)alkyl). In some embodiments, R^{A} represents H.

In certain embodiments, the compound of formula (II) is selected from the following table of compounds, and pharmaceutically acceptable salts thereof:

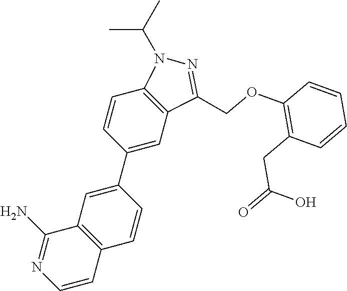 [SMILES: CC(C)n1nc(COc2ccccc2CC(=O)O)c2cc(-c3ccc4ccnc(N)c4c3)ccc21]

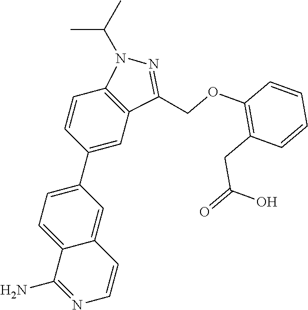 [SMILES: CC(C)n1nc(COc2ccccc2CC(=O)O)c2cc(-c3ccc4c(N)nccc4c3)ccc21]

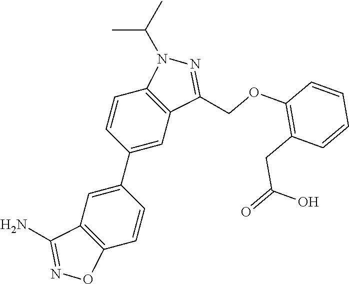 [SMILES: CC(C)n1nc(COc2ccccc2CC(=O)O)c2cc(-c3ccc4onc(N)c4c3)ccc21]

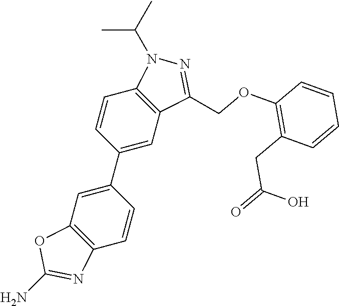 [SMILES: CC(C)n1nc(COc2ccccc2CC(=O)O)c2cc(-c3ccc4nc(N)oc4c3)ccc21]

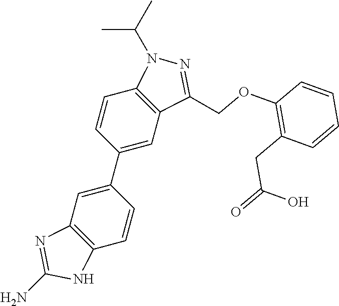 [SMILES: CC(C)n1nc(COc2ccccc2CC(=O)O)c2cc(-c3ccc4[nH]c(N)nc4c3)ccc21]

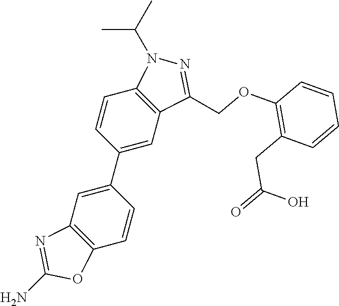 [SMILES: CC(C)n1nc(COc2ccccc2CC(=O)O)c2cc(-c3ccc4oc(N)nc4c3)ccc21]

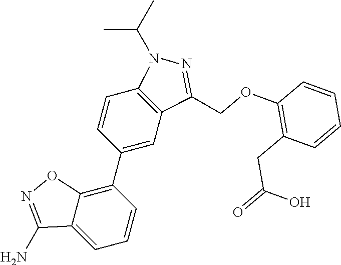 [SMILES: CC(C)n1nc(COc2ccccc2CC(=O)O)c2cc(-c3cccc4c(N)noc34)ccc21]

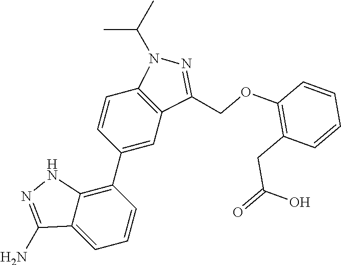 [SMILES: CC(C)n1nc(COc2ccccc2CC(=O)O)c2cc(-c3cccc4c(N)n[nH]c34)ccc21]

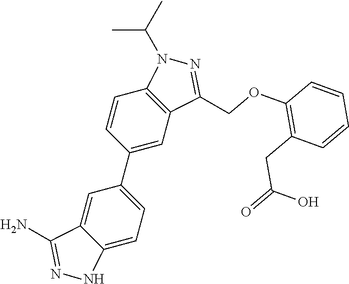 [SMILES: CC(C)n1nc(COc2ccccc2CC(=O)O)c2cc(-c3ccc4[nH]nc(N)c4c3)ccc21]

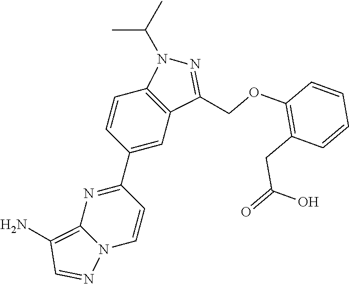 [SMILES: CC(C)n1nc(COc2ccccc2CC(=O)O)c2cc(-c3ccn4ncc(N)c4n3)ccc21]

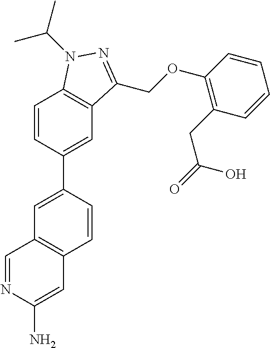 [SMILES: CC(C)n1nc(COc2ccccc2CC(=O)O)c2cc(-c3ccc4cc(N)ncc4c3)ccc21]

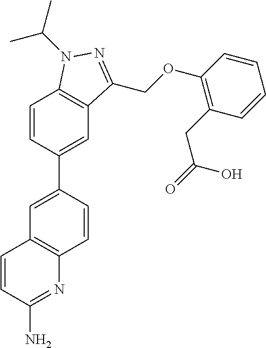 [SMILES: CC(C)n1nc(COc2ccccc2CC(=O)O)c2cc(-c3ccc4nc(N)ccc4c3)ccc21]

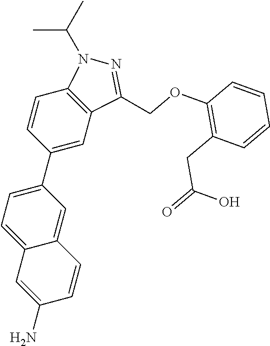 [SMILES: CC(C)n1nc(COc2ccccc2CC(=O)O)c2cc(-c3ccc4cc(N)ccc4c3)ccc21]

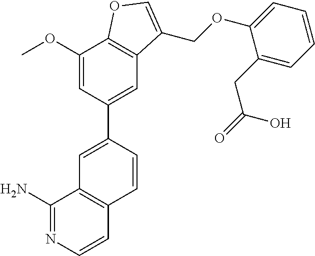 [SMILES: COc1cc(-c2ccc3ccnc(N)c3c2)cc2c(COc3ccccc3CC(=O)O)coc12]

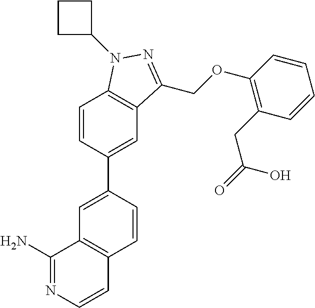 [SMILES: Nc1nccc2ccc(-c3ccc4c(c3)c(COc3ccccc3CC(=O)O)nn4C3CCC3)cc12]

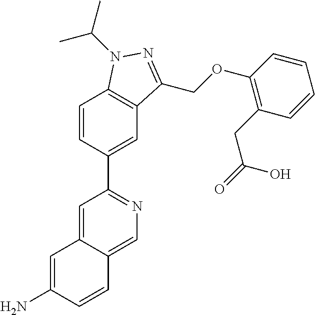 [SMILES: CC(C)n1nc(COc2ccccc2CC(=O)O)c2cc(-c3cc4cc(N)ccc4cn3)ccc21]

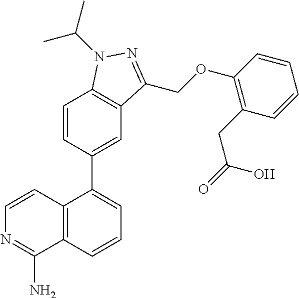 [SMILES: CC(C)n1nc(COc2ccccc2CC(=O)O)c2cc(-c3cccc4c(N)nccc34)ccc21]

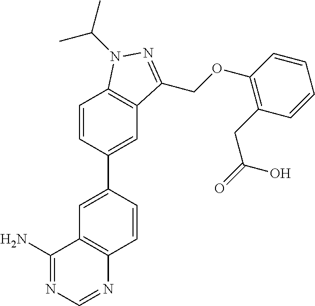 [SMILES: CC(C)n1nc(COc2ccccc2CC(=O)O)c2cc(-c3ccc4ncnc(N)c4c3)ccc21]

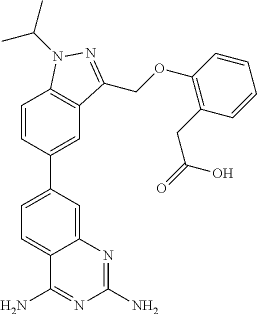 [SMILES: CC(C)n1nc(COc2ccccc2CC(=O)O)c2cc(-c3ccc4c(N)nc(N)nc4c3)ccc21]

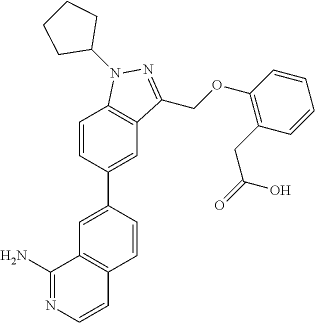 [SMILES: Nc1nccc2ccc(-c3ccc4c(c3)c(COc3ccccc3CC(=O)O)nn4C3CCCC3)cc12]

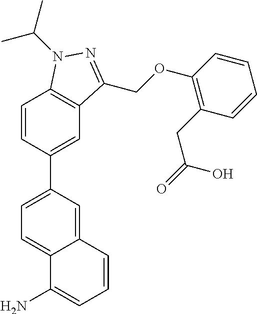 [SMILES: CC(C)n1nc(COc2ccccc2CC(=O)O)c2cc(-c3ccc4c(N)cccc4c3)ccc21]

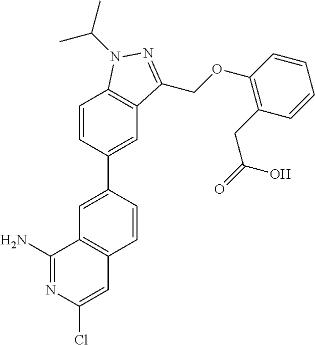 [SMILES: CC(C)n1nc(COc2ccccc2CC(=O)O)c2cc(-c3ccc4cc(Cl)nc(N)c4c3)ccc21]

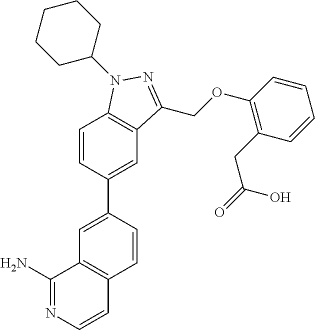 [SMILES: Nc1nccc2ccc(-c3ccc4c(c3)c(COc3ccccc3CC(=O)O)nn4C3CCCCC3)cc12]

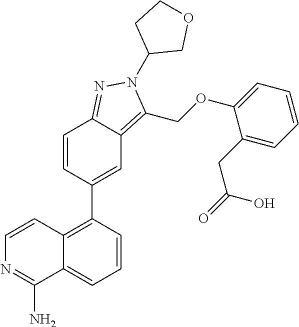 [SMILES: Nc1nccc2c(-c3ccc4nn(C5CCOC5)c(COc5ccccc5CC(=O)O)c4c3)cccc12]

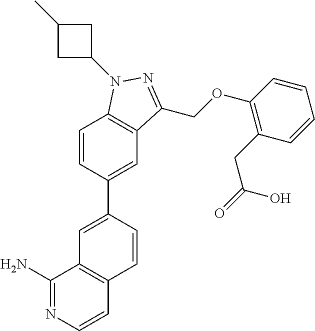 [SMILES: CC1CC(n2nc(COc3ccccc3CC(=O)O)c3cc(-c4ccc5ccnc(N)c5c4)ccc32)C1]

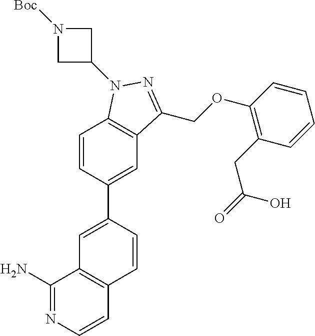 [SMILES: CC(C)(C)OC(=O)N1CC(n2nc(COc3ccccc3CC(=O)O)c3cc(-c4ccc5ccnc(N)c5c4)ccc32)C1]

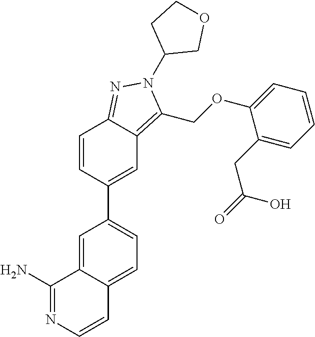 [SMILES: Nc1nccc2ccc(-c3ccc4nn(C5CCOC5)c(COc5ccccc5CC(=O)O)c4c3)cc12]

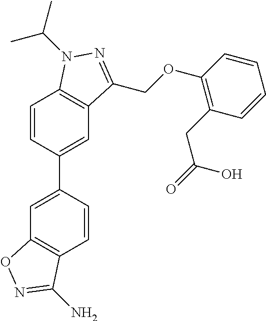 [SMILES: CC(C)n1nc(COc2ccccc2CC(=O)O)c2cc(-c3ccc4c(N)noc4c3)ccc21]

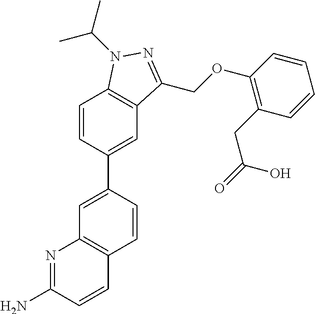 [SMILES: CC(C)n1nc(COc2ccccc2CC(=O)O)c2cc(-c3ccc4ccc(N)nc4c3)ccc21]

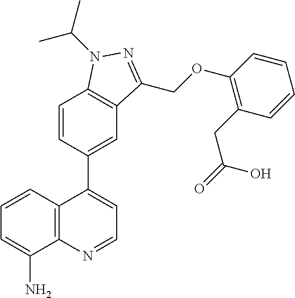 [SMILES: CC(C)n1nc(COc2ccccc2CC(=O)O)c2cc(-c3ccnc4c(N)cccc34)ccc21]

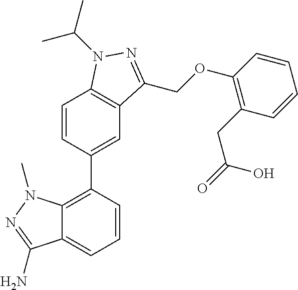 [SMILES: CC(C)n1nc(COc2ccccc2CC(=O)O)c2cc(-c3cccc4c(N)nn(C)c34)ccc21]

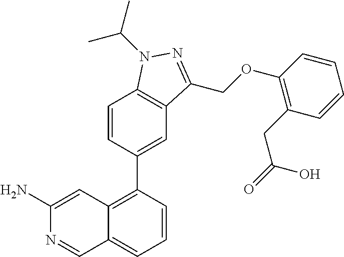 [SMILES: CC(C)n1nc(COc2ccccc2CC(=O)O)c2cc(-c3cccc4cnc(N)cc34)ccc21]

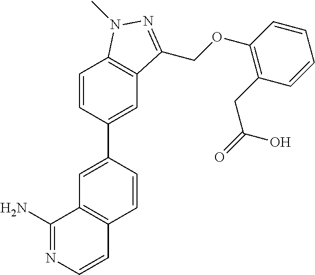 [SMILES: Cn1nc(COc2ccccc2CC(=O)O)c2cc(-c3ccc4ccnc(N)c4c3)ccc21]

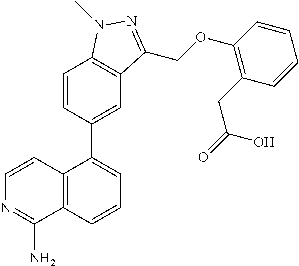 [SMILES: Cn1nc(COc2ccccc2CC(=O)O)c2cc(-c3cccc4c(N)nccc34)ccc21]

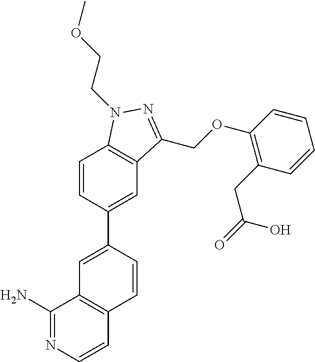 [SMILES: COCCn1nc(COc2ccccc2CC(=O)O)c2cc(-c3ccc4ccnc(N)c4c3)ccc21]

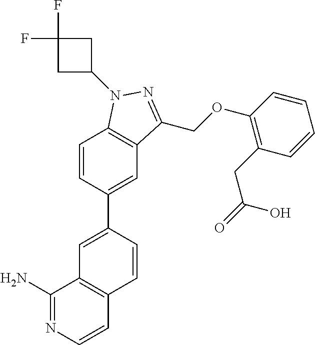 [SMILES: Nc1nccc2ccc(-c3ccc4c(c3)c(COc3ccccc3CC(=O)O)nn4C3CC(F)(F)C3)cc12]

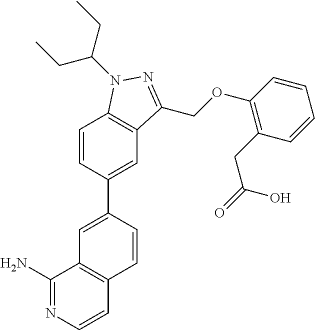 [SMILES: CCC(CC)n1nc(COc2ccccc2CC(=O)O)c2cc(-c3ccc4ccnc(N)c4c3)ccc21]

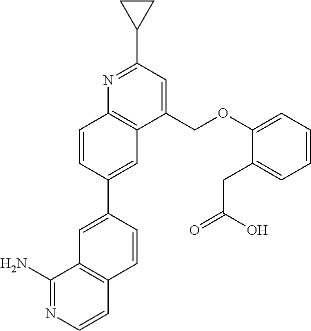 [SMILES: Nc1nccc2ccc(-c3ccc4nc(C5CC5)cc(COc5ccccc5CC(=O)O)c4c3)cc12]

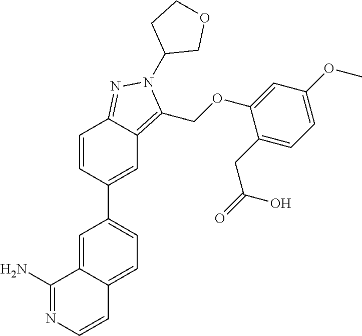 [SMILES: COc1ccc(CC(=O)O)c(OCc2c3cc(-c4ccc5ccnc(N)c5c4)ccc3nn2C2CCOC2)c1]

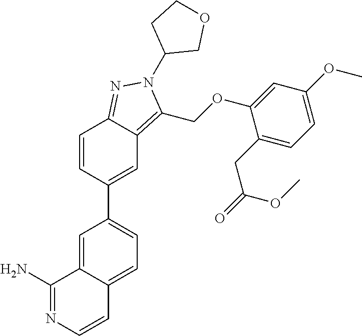 [SMILES: COC(=O)Cc1ccc(OC)cc1OCc1c2cc(-c3ccc4ccnc(N)c4c3)ccc2nn1C1CCOC1]

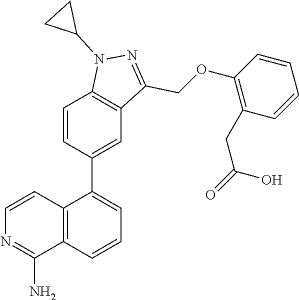 [SMILES: Nc1nccc2c(-c3ccc4c(c3)c(COc3ccccc3CC(=O)O)nn4C3CC3)cccc12]

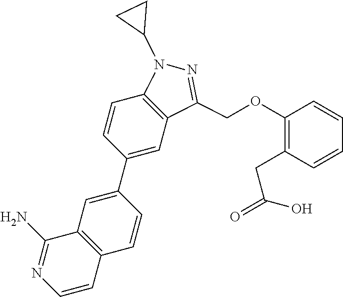 [SMILES: Nc1nccc2ccc(-c3ccc4c(c3)c(COc3ccccc3CC(=O)O)nn4C3CC3)cc12]

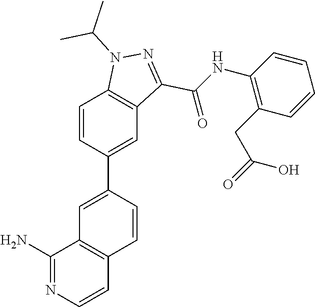 [SMILES: CC(C)n1nc(C(=O)Nc2ccccc2CC(=O)O)c2cc(-c3ccc4ccnc(N)c4c3)ccc21]

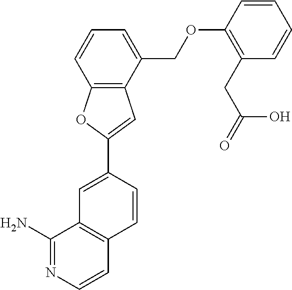 [SMILES: Nc1nccc2ccc(-c3cc4c(COc5ccccc5CC(=O)O)cccc4o3)cc12]

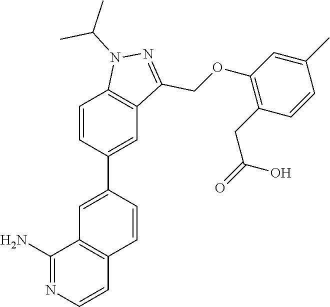 [SMILES: Cc1ccc(CC(=O)O)c(OCc2nn(C(C)C)c3ccc(-c4ccc5ccnc(N)c5c4)cc23)c1]

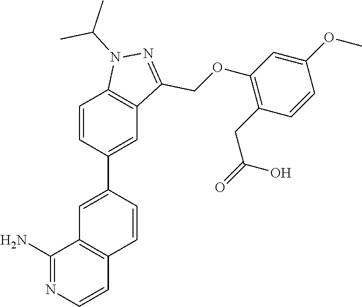 [SMILES: COc1ccc(CC(=O)O)c(OCc2nn(C(C)C)c3ccc(-c4ccc5ccnc(N)c5c4)cc23)c1]

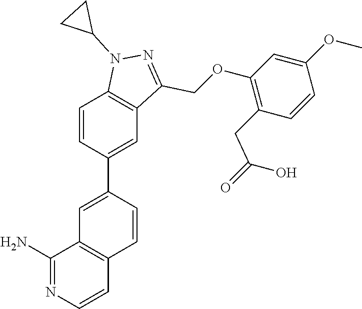 [SMILES: COc1ccc(CC(=O)O)c(OCc2nn(C3CC3)c3ccc(-c4ccc5ccnc(N)c5c4)cc23)c1]

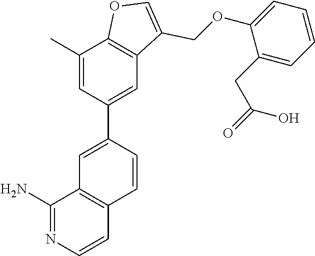 [SMILES: Cc1cc(-c2ccc3ccnc(N)c3c2)cc2c(COc3ccccc3CC(=O)O)coc12]

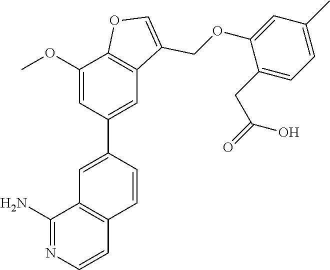 [SMILES: COc1cc(-c2ccc3ccnc(N)c3c2)cc2c(COc3cc(C)ccc3CC(=O)O)coc12]

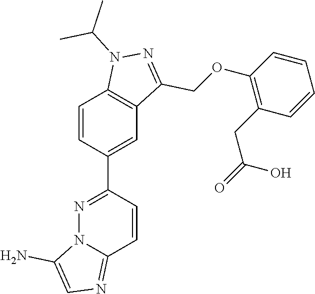 [SMILES: CC(C)n1nc(COc2ccccc2CC(=O)O)c2cc(-c3ccc4ncc(N)n4n3)ccc21]

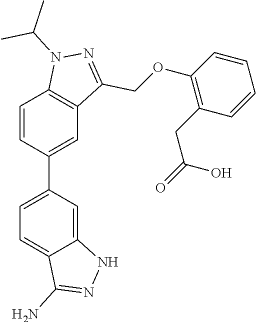 [SMILES: CC(C)n1nc(COc2ccccc2CC(=O)O)c2cc(-c3ccc4c(N)n[nH]c4c3)ccc21]

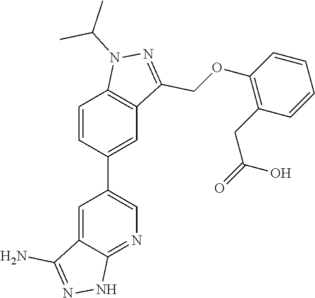 [SMILES: CC(C)n1nc(COc2ccccc2CC(=O)O)c2cc(-c3cnc4[nH]nc(N)c4c3)ccc21]

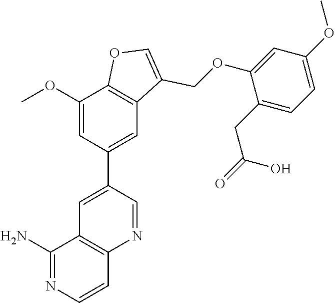 [SMILES: COc1ccc(CC(=O)O)c(OCc2coc3c(OC)cc(-c4cnc5ccnc(N)c5c4)cc23)c1]

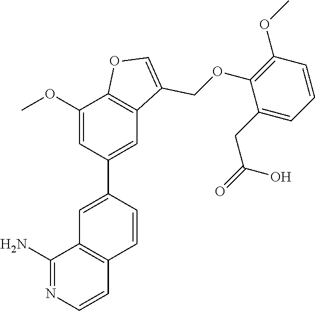 [SMILES: COc1cccc(CC(=O)O)c1OCc1coc2c(OC)cc(-c3ccc4ccnc(N)c4c3)cc12]

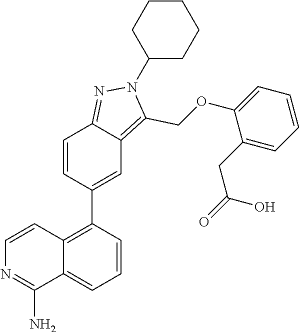 [SMILES: Nc1nccc2c(-c3ccc4nn(C5CCCCC5)c(COc5ccccc5CC(=O)O)c4c3)cccc12]

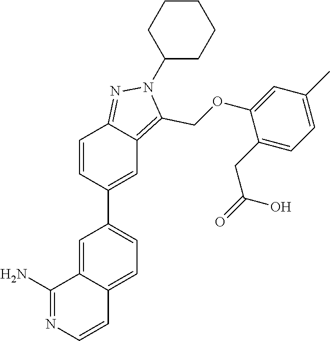 [SMILES: Cc1ccc(CC(=O)O)c(OCc2c3cc(-c4ccc5ccnc(N)c5c4)ccc3nn2C2CCCCC2)c1]

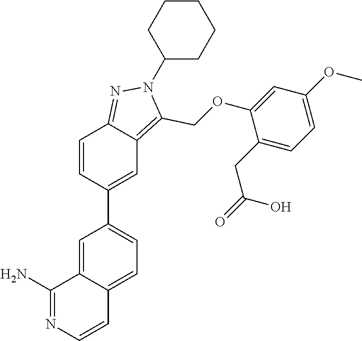 [SMILES: COc1ccc(CC(=O)O)c(OCc2c3cc(-c4ccc5ccnc(N)c5c4)ccc3nn2C2CCCCC2)c1]

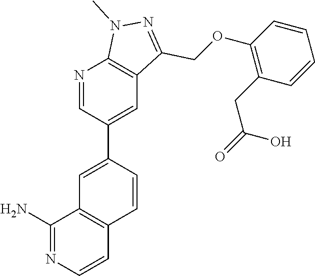 [SMILES: Cn1nc(COc2ccccc2CC(=O)O)c2cc(-c3ccc4ccnc(N)c4c3)cnc21]

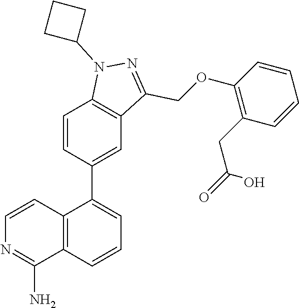 [SMILES: Nc1nccc2c(-c3ccc4c(c3)c(COc3ccccc3CC(=O)O)nn4C3CCC3)cccc12]

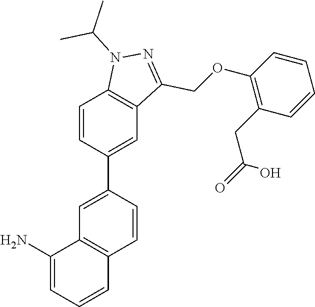 [SMILES: CC(C)n1nc(COc2ccccc2CC(=O)O)c2cc(-c3ccc4cccc(N)c4c3)ccc21]

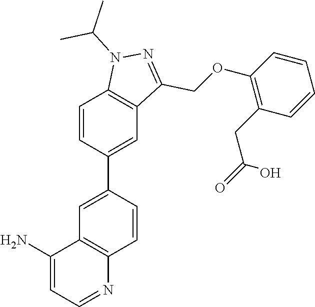 [SMILES: CC(C)n1nc(COc2ccccc2CC(=O)O)c2cc(-c3ccc4nccc(N)c4c3)ccc21]

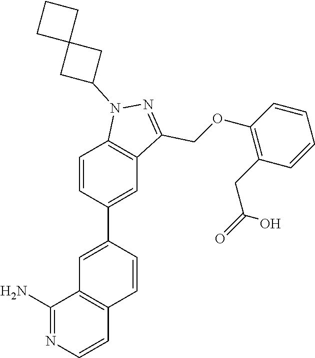 [SMILES: Nc1nccc2ccc(-c3ccc4c(c3)c(COc3ccccc3CC(=O)O)nn4C3CC4(CCC4)C3)cc12]

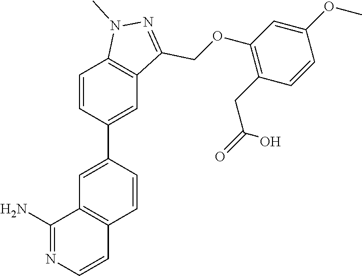 [SMILES: COc1ccc(CC(=O)O)c(OCc2nn(C)c3ccc(-c4ccc5ccnc(N)c5c4)cc23)c1]

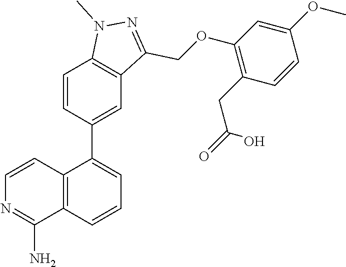 [SMILES: COc1ccc(CC(=O)O)c(OCc2nn(C)c3ccc(-c4cccc5c(N)nccc45)cc23)c1]

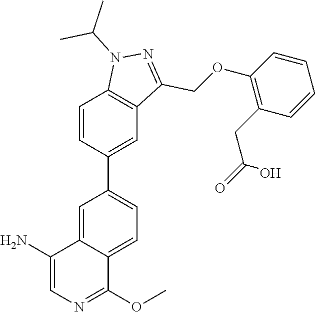 [SMILES: COc1ncc(N)c2cc(-c3ccc4c(c3)c(COc3ccccc3CC(=O)O)nn4C(C)C)ccc12]

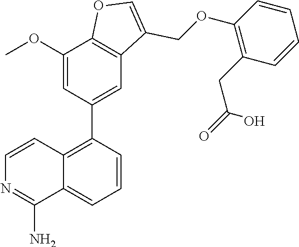 [SMILES: COc1cc(-c2cccc3c(N)nccc23)cc2c(COc3ccccc3CC(=O)O)coc12]

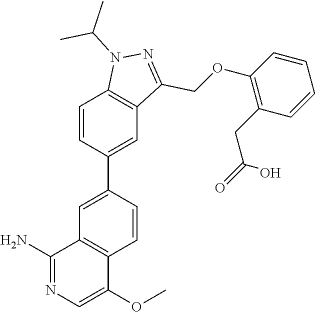 [SMILES: COc1cnc(N)c2cc(-c3ccc4c(c3)c(COc3ccccc3CC(=O)O)nn4C(C)C)ccc12]

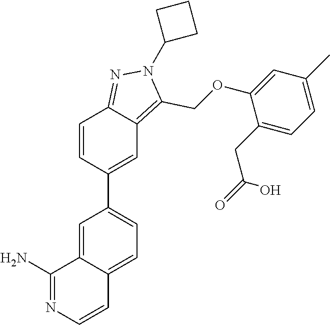 [SMILES: Cc1ccc(CC(=O)O)c(OCc2c3cc(-c4ccc5ccnc(N)c5c4)ccc3nn2C2CCC2)c1]

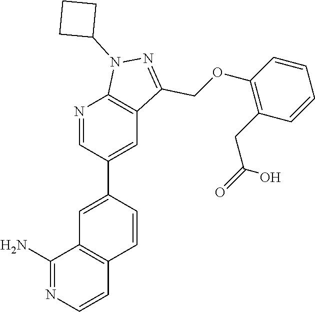 [SMILES: Nc1nccc2ccc(-c3cnc4c(c3)c(COc3ccccc3CC(=O)O)nn4C3CCC3)cc12]

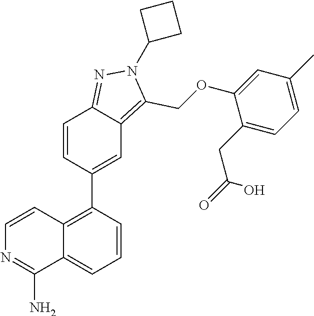 [SMILES: Cc1ccc(CC(=O)O)c(OCc2c3cc(-c4cccc5c(N)nccc45)ccc3nn2C2CCC2)c1]

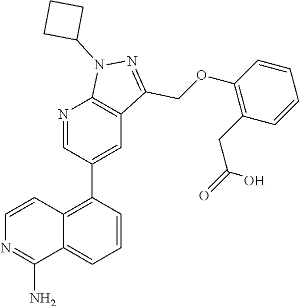 [SMILES: Nc1nccc2c(-c3cnc4c(c3)c(COc3ccccc3CC(=O)O)nn4C3CCC3)cccc12]

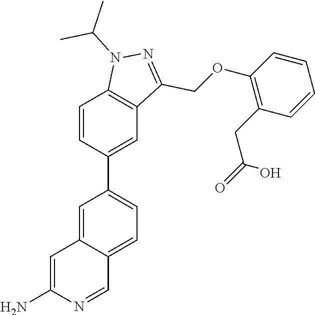 [SMILES: CC(C)n1nc(COc2ccccc2CC(=O)O)c2cc(-c3ccc4cnc(N)cc4c3)ccc21]

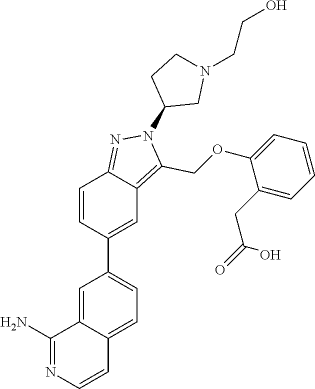 [SMILES: Nc1nccc2ccc(-c3ccc4nn([C@H]5CCN(CCO)C5)c(COc5ccccc5CC(=O)O)c4c3)cc12]

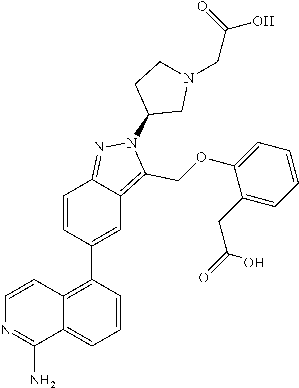 [SMILES: Nc1nccc2c(-c3ccc4nn([C@H]5CCN(CC(=O)O)C5)c(COc5ccccc5CC(=O)O)c4c3)cccc12]

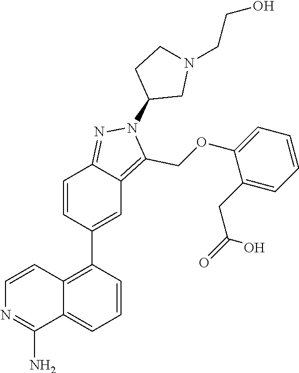 [SMILES: Nc1nccc2c(-c3ccc4nn([C@H]5CCN(CCO)C5)c(COc5ccccc5CC(=O)O)c4c3)cccc12]

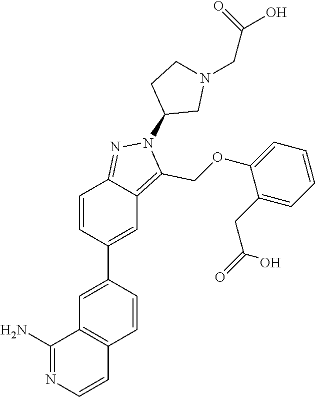 [SMILES: Nc1nccc2ccc(-c3ccc4nn([C@H]5CCN(CC(=O)O)C5)c(COc5ccccc5CC(=O)O)c4c3)cc12]

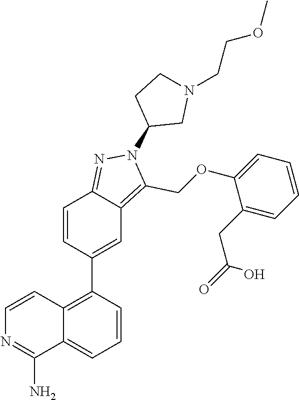 [SMILES: COCCN1CC[C@H](n2nc3ccc(-c4cccc5c(N)nccc45)cc3c2COc2ccccc2CC(=O)O)C1]

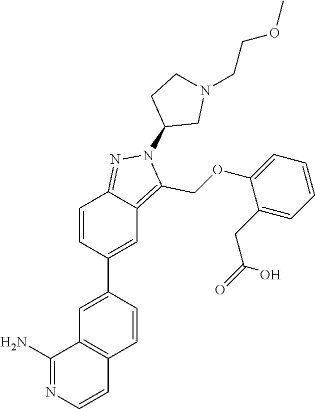 [SMILES: COCCN1CC[C@H](n2nc3ccc(-c4ccc5ccnc(N)c5c4)cc3c2COc2ccccc2CC(=O)O)C1]

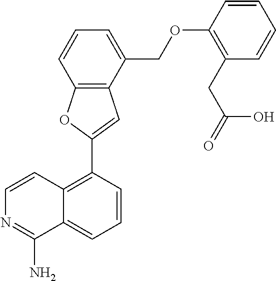 [SMILES: Nc1nccc2c(-c3cc4c(COc5ccccc5CC(=O)O)cccc4o3)cccc12]

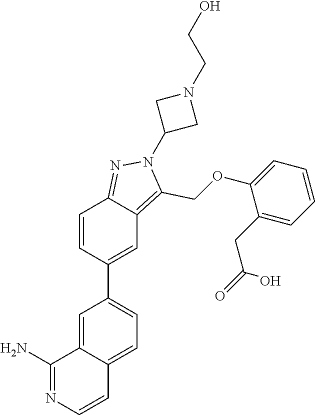 [SMILES: Nc1nccc2ccc(-c3ccc4nn(C5CN(CCO)C5)c(COc5ccccc5CC(=O)O)c4c3)cc12]

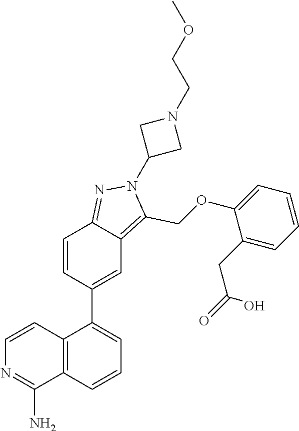 [SMILES: COCCN1CC(n2nc3ccc(-c4cccc5c(N)nccc45)cc3c2COc2ccccc2CC(=O)O)C1]

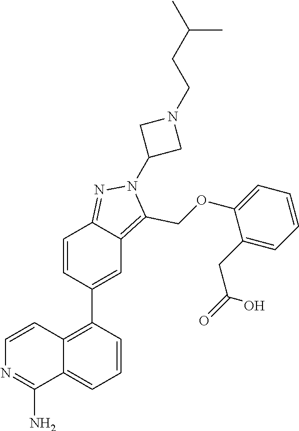 [SMILES: CC(C)CCN1CC(n2nc3ccc(-c4cccc5c(N)nccc45)cc3c2COc2ccccc2CC(=O)O)C1]

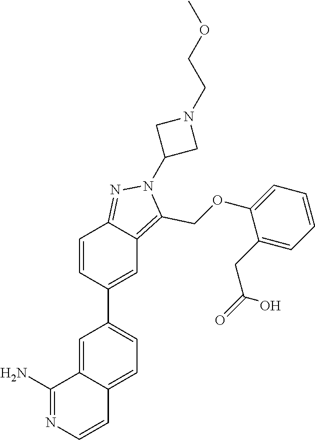 [SMILES: COCCN1CC(n2nc3ccc(-c4ccc5ccnc(N)c5c4)cc3c2COc2ccccc2CC(=O)O)C1]

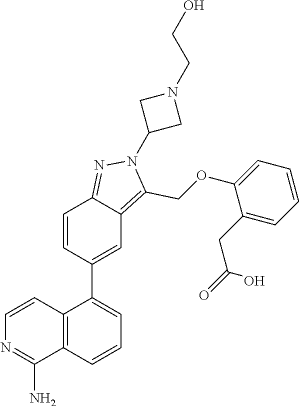 [SMILES: Nc1nccc2c(-c3ccc4nn(C5CN(CCO)C5)c(COc5ccccc5CC(=O)O)c4c3)cccc12]

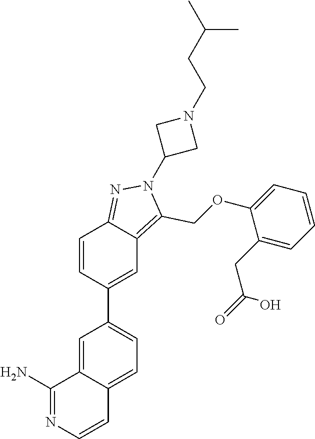 [SMILES: CC(C)CCN1CC(n2nc3ccc(-c4ccc5ccnc(N)c5c4)cc3c2COc2ccccc2CC(=O)O)C1]

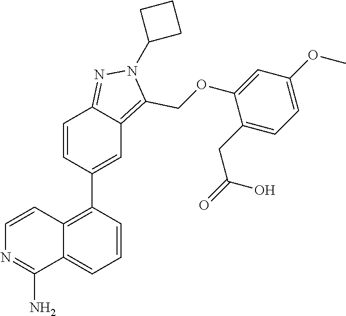 [SMILES: COc1ccc(CC(=O)O)c(OCc2c3cc(-c4cccc5c(N)nccc45)ccc3nn2C2CCC2)c1]

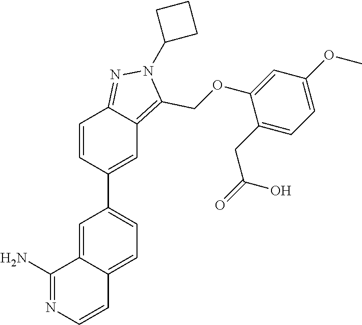 [SMILES: COc1ccc(CC(=O)O)c(OCc2c3cc(-c4ccc5ccnc(N)c5c4)ccc3nn2C2CCC2)c1]

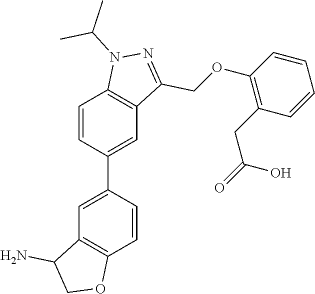 [SMILES: CC(C)n1nc(COc2ccccc2CC(=O)O)c2cc(-c3ccc4c(c3)C(N)CO4)ccc21]

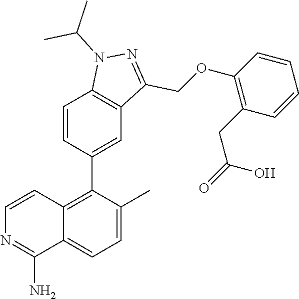 [SMILES: Cc1ccc2c(N)nccc2c1-c1ccc2c(c1)c(COc1ccccc1CC(=O)O)nn2C(C)C]

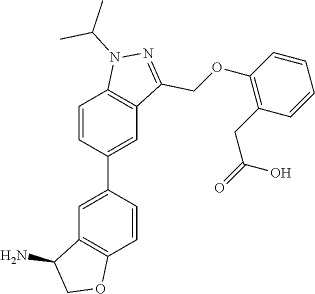 [SMILES: CC(C)n1nc(COc2ccccc2CC(=O)O)c2cc(-c3ccc4c(c3)[C@H](N)CO4)ccc21]

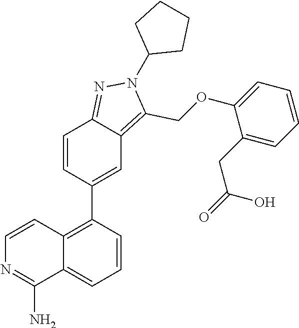 [SMILES: Nc1nccc2c(-c3ccc4nn(C5CCCC5)c(COc5ccccc5CC(=O)O)c4c3)cccc12]

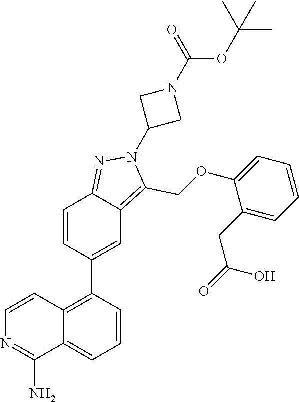 [SMILES: CC(C)(C)OC(=O)N1CC(n2nc3ccc(-c4cccc5c(N)nccc45)cc3c2COc2ccccc2CC(=O)O)C1]

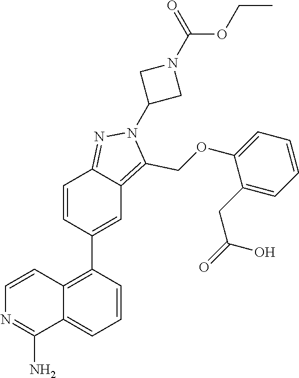 [SMILES: CCOC(=O)N1CC(n2nc3ccc(-c4cccc5c(N)nccc45)cc3c2COc2ccccc2CC(=O)O)C1]

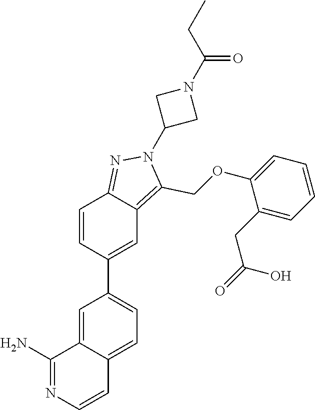 [SMILES: CCC(=O)N1CC(n2nc3ccc(-c4ccc5ccnc(N)c5c4)cc3c2COc2ccccc2CC(=O)O)C1]

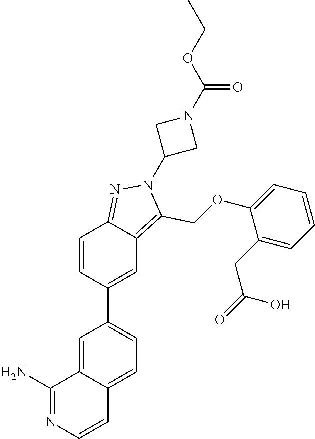 [SMILES: CCOC(=O)N1CC(n2nc3ccc(-c4ccc5ccnc(N)c5c4)cc3c2COc2ccccc2CC(=O)O)C1]

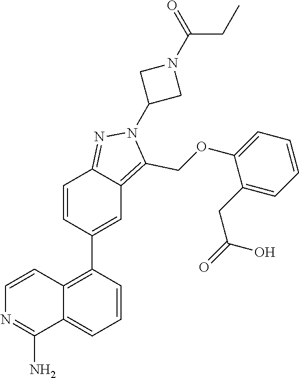 [SMILES: CCC(=O)N1CC(n2nc3ccc(-c4cccc5c(N)nccc45)cc3c2COc2ccccc2CC(=O)O)C1]

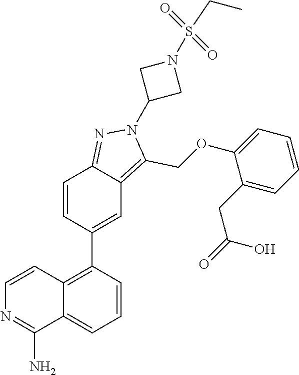 [SMILES: CCS(=O)(=O)N1CC(n2nc3ccc(-c4cccc5c(N)nccc45)cc3c2COc2ccccc2CC(=O)O)C1]

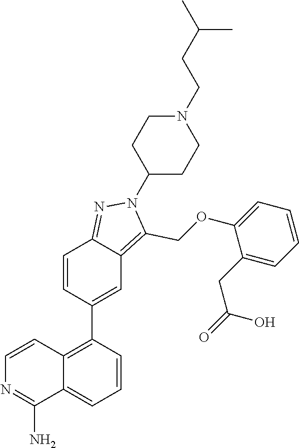 [SMILES: CC(C)CCN1CCC(n2nc3ccc(-c4cccc5c(N)nccc45)cc3c2COc2ccccc2CC(=O)O)CC1]

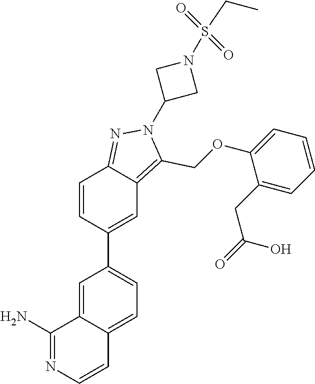 [SMILES: CCS(=O)(=O)N1CC(n2nc3ccc(-c4ccc5ccnc(N)c5c4)cc3c2COc2ccccc2CC(=O)O)C1]

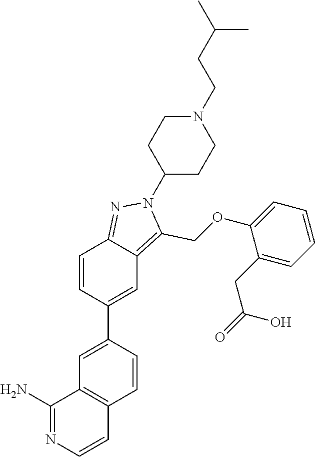 [SMILES: CC(C)CCN1CCC(n2nc3ccc(-c4ccc5ccnc(N)c5c4)cc3c2COc2ccccc2CC(=O)O)CC1]

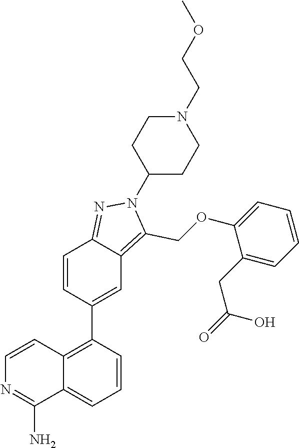 [SMILES: COCCN1CCC(n2nc3ccc(-c4cccc5c(N)nccc45)cc3c2COc2ccccc2CC(=O)O)CC1]

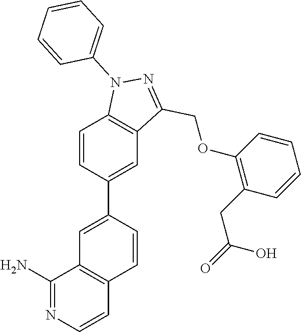 [SMILES: Nc1nccc2ccc(-c3ccc4c(c3)c(COc3ccccc3CC(=O)O)nn4-c3ccccc3)cc12]

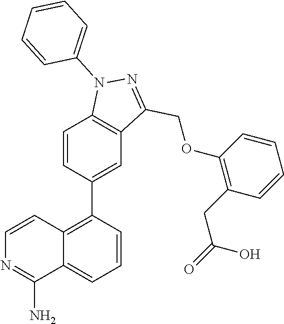 [SMILES: Nc1nccc2c(-c3ccc4c(c3)c(COc3ccccc3CC(=O)O)nn4-c3ccccc3)cccc12]

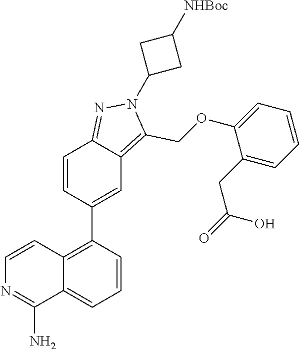 [SMILES: CC(C)(C)OC(=O)NC1CC(n2nc3ccc(-c4cccc5c(N)nccc45)cc3c2COc2ccccc2CC(=O)O)C1]

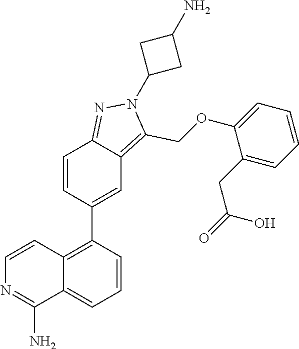 [SMILES: Nc1nccc2c(-c3ccc4nn(C5CC(N)C5)c(COc5ccccc5CC(=O)O)c4c3)cccc12]

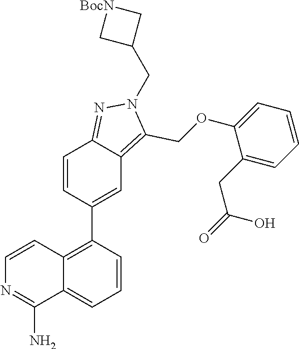 [SMILES: CC(C)(C)OC(=O)N1CC(Cn2nc3ccc(-c4cccc5c(N)nccc45)cc3c2COc2ccccc2CC(=O)O)C1]

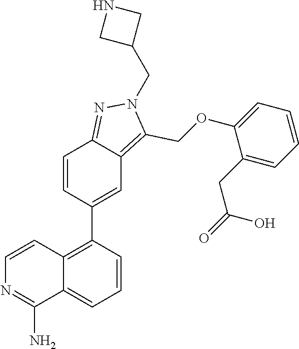 [SMILES: Nc1nccc2c(-c3ccc4nn(CC5CNC5)c(COc5ccccc5CC(=O)O)c4c3)cccc12]

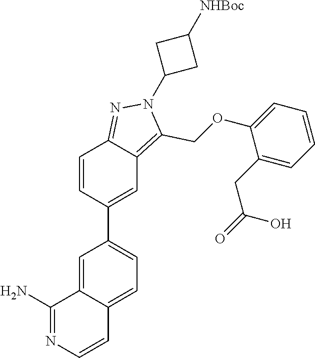 [SMILES: CC(C)(C)OC(=O)NC1CC(n2nc3ccc(-c4ccc5ccnc(N)c5c4)cc3c2COc2ccccc2CC(=O)O)C1]

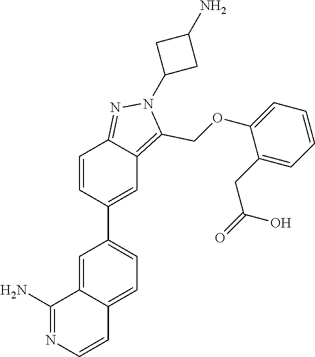 [SMILES: Nc1nccc2ccc(-c3ccc4nn(C5CC(N)C5)c(COc5ccccc5CC(=O)O)c4c3)cc12]

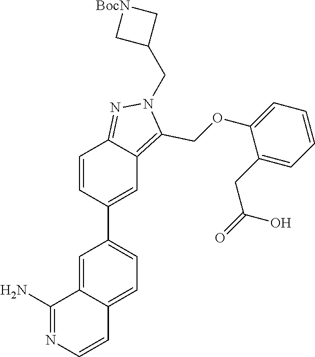 [SMILES: CC(C)(C)OC(=O)N1CC(Cn2nc3ccc(-c4ccc5ccnc(N)c5c4)cc3c2COc2ccccc2CC(=O)O)C1]

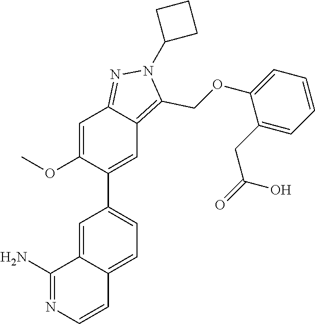 [SMILES: COc1cc2nn(C3CCC3)c(COc3ccccc3CC(=O)O)c2cc1-c1ccc2ccnc(N)c2c1]

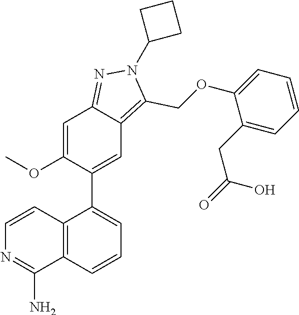 [SMILES: COc1cc2nn(C3CCC3)c(COc3ccccc3CC(=O)O)c2cc1-c1cccc2c(N)nccc12]

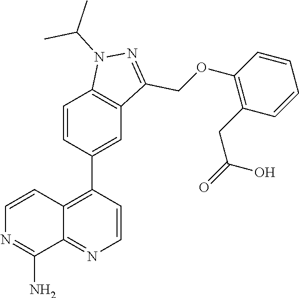 [SMILES: CC(C)n1nc(COc2ccccc2CC(=O)O)c2cc(-c3ccnc4c(N)nccc34)ccc21]

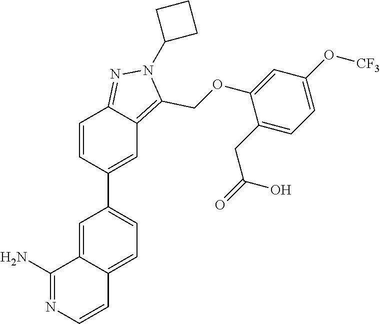 [SMILES: Nc1nccc2ccc(-c3ccc4nn(C5CCC5)c(COc5cc(OC(F)(F)F)ccc5CC(=O)O)c4c3)cc12]

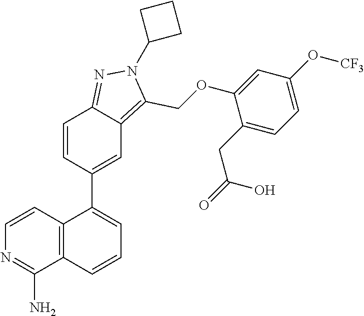 [SMILES: Nc1nccc2c(-c3ccc4nn(C5CCC5)c(COc5cc(OC(F)(F)F)ccc5CC(=O)O)c4c3)cccc12]

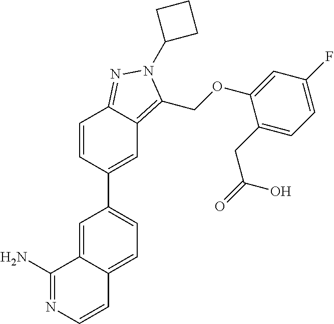 [SMILES: Nc1nccc2ccc(-c3ccc4nn(C5CCC5)c(COc5cc(F)ccc5CC(=O)O)c4c3)cc12]

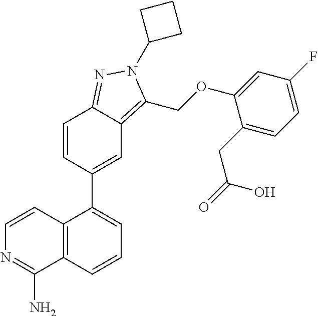 [SMILES: Nc1nccc2c(-c3ccc4nn(C5CCC5)c(COc5cc(F)ccc5CC(=O)O)c4c3)cccc12]

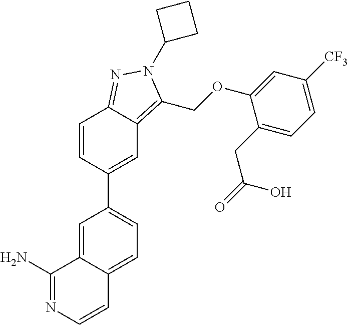 [SMILES: Nc1nccc2ccc(-c3ccc4nn(C5CCC5)c(COc5cc(C(F)(F)F)ccc5CC(=O)O)c4c3)cc12]

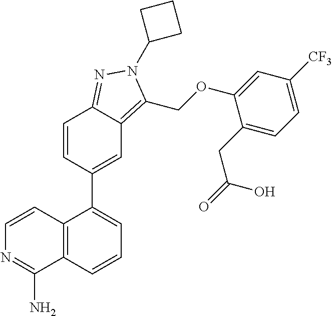 [SMILES: Nc1nccc2c(-c3ccc4nn(C5CCC5)c(COc5cc(C(F)(F)F)ccc5CC(=O)O)c4c3)cccc12]

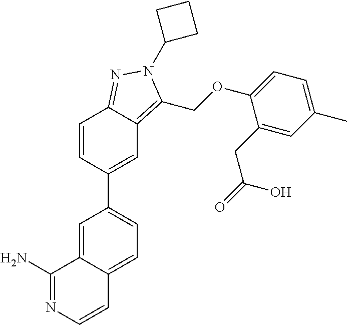 [SMILES: Cc1ccc(OCc2c3cc(-c4ccc5ccnc(N)c5c4)ccc3nn2C2CCC2)c(CC(=O)O)c1]

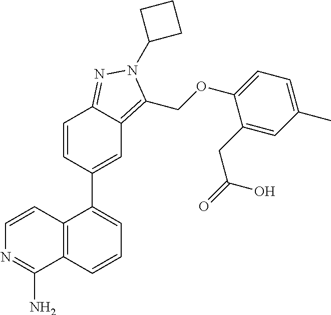 [SMILES: Cc1ccc(OCc2c3cc(-c4cccc5c(N)nccc45)ccc3nn2C2CCC2)c(CC(=O)O)c1]

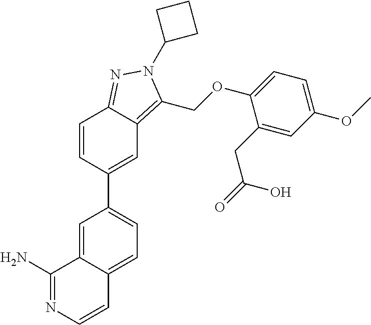 [SMILES: COc1ccc(OCc2c3cc(-c4ccc5ccnc(N)c5c4)ccc3nn2C2CCC2)c(CC(=O)O)c1]

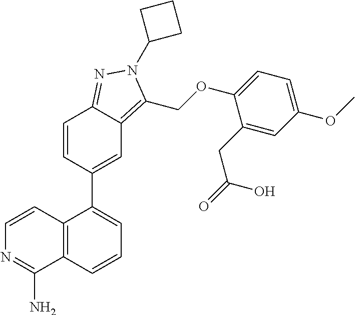 [SMILES: COc1ccc(OCc2c3cc(-c4cccc5c(N)nccc45)ccc3nn2C2CCC2)c(CC(=O)O)c1]

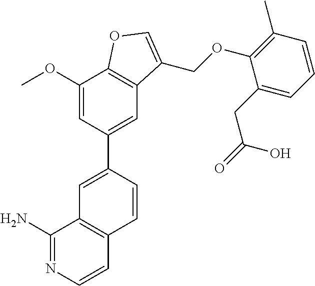 [SMILES: COc1cc(-c2ccc3ccnc(N)c3c2)cc2c(COc3c(C)cccc3CC(=O)O)coc12]

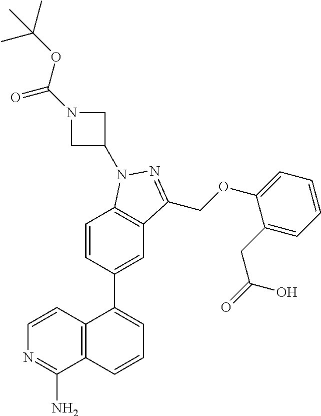 [SMILES: CC(C)(C)OC(=O)N1CC(n2nc(COc3ccccc3CC(=O)O)c3cc(-c4cccc5c(N)nccc45)ccc32)C1]

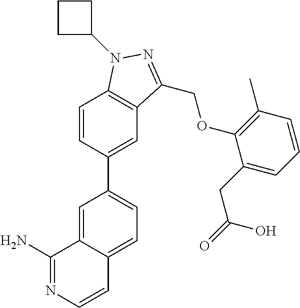 [SMILES: Cc1cccc(CC(=O)O)c1OCc1nn(C2CCC2)c2ccc(-c3ccc4ccnc(N)c4c3)cc12]

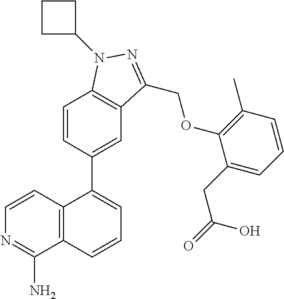 [SMILES: Cc1cccc(CC(=O)O)c1OCc1nn(C2CCC2)c2ccc(-c3cccc4c(N)nccc34)cc12]

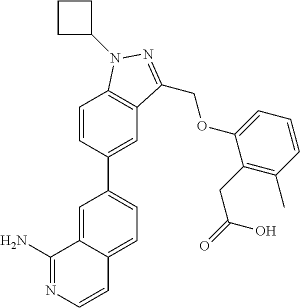 [SMILES: Cc1cccc(OCc2nn(C3CCC3)c3ccc(-c4ccc5ccnc(N)c5c4)cc23)c1CC(=O)O]

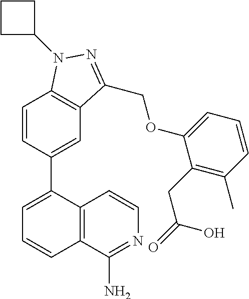 [SMILES: Cc1cccc(OCc2nn(C3CCC3)c3ccc(-c4cccc5c(N)nccc45)cc23)c1CC(=O)O]

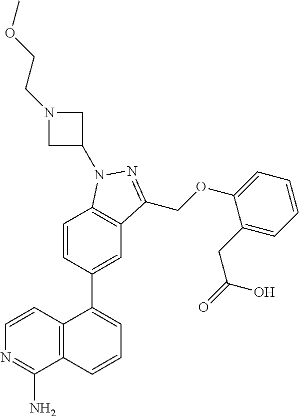 [SMILES: COCCN1CC(n2nc(COc3ccccc3CC(=O)O)c3cc(-c4cccc5c(N)nccc45)ccc32)C1]

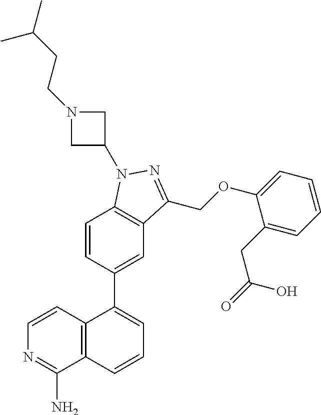 [SMILES: CC(C)CCN1CC(n2nc(COc3ccccc3CC(=O)O)c3cc(-c4cccc5c(N)nccc45)ccc32)C1]

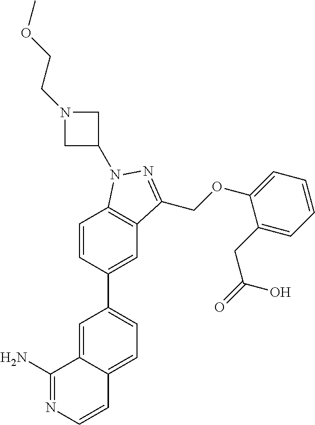 [SMILES: COCCN1CC(n2nc(COc3ccccc3CC(=O)O)c3cc(-c4ccc5ccnc(N)c5c4)ccc32)C1]

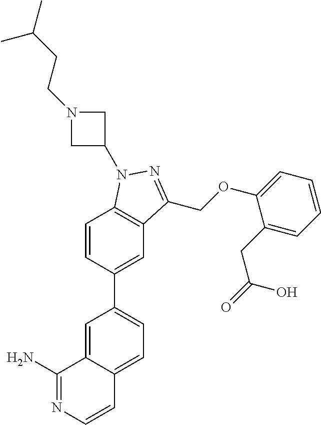 [SMILES: CC(C)CCN1CC(n2nc(COc3ccccc3CC(=O)O)c3cc(-c4ccc5ccnc(N)c5c4)ccc32)C1]

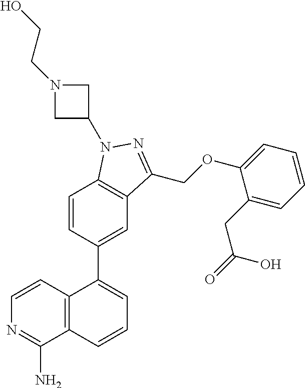 [SMILES: Nc1nccc2c(-c3ccc4c(c3)c(COc3ccccc3CC(=O)O)nn4C3CN(CCO)C3)cccc12]

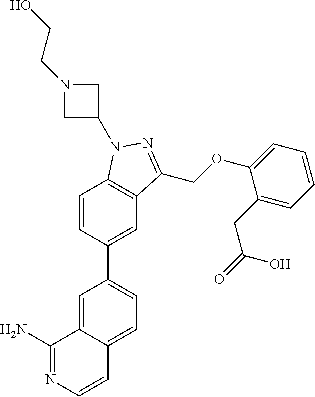 [SMILES: Nc1nccc2ccc(-c3ccc4c(c3)c(COc3ccccc3CC(=O)O)nn4C3CN(CCO)C3)cc12]

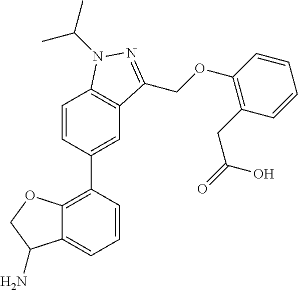 [SMILES: CC(C)n1nc(COc2ccccc2CC(=O)O)c2cc(-c3cccc4c3OCC4N)ccc21]

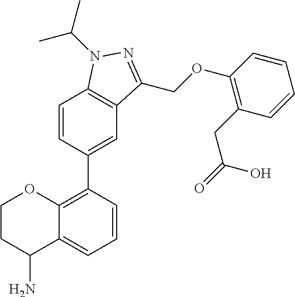 [SMILES: CC(C)n1nc(COc2ccccc2CC(=O)O)c2cc(-c3cccc4c3OCCC4N)ccc21]

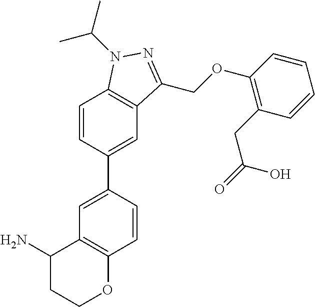 [SMILES: CC(C)n1nc(COc2ccccc2CC(=O)O)c2cc(-c3ccc4c(c3)C(N)CCO4)ccc21]

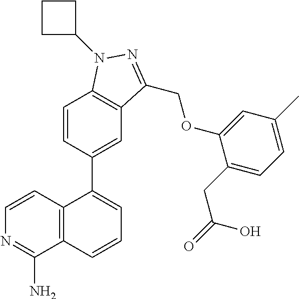 [SMILES: Cc1ccc(CC(=O)O)c(OCc2nn(C3CCC3)c3ccc(-c4cccc5c(N)nccc45)cc23)c1]

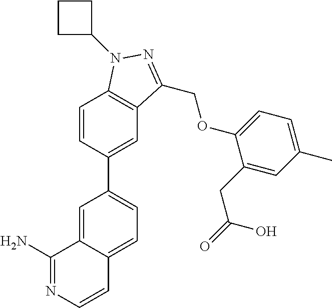 [SMILES: Cc1ccc(OCc2nn(C3CCC3)c3ccc(-c4ccc5ccnc(N)c5c4)cc23)c(CC(=O)O)c1]

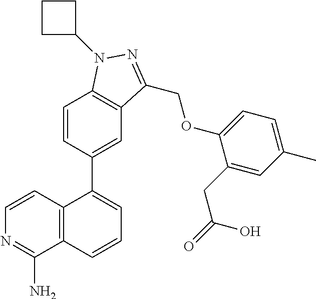 [SMILES: Cc1ccc(OCc2nn(C3CCC3)c3ccc(-c4cccc5c(N)nccc45)cc23)c(CC(=O)O)c1]

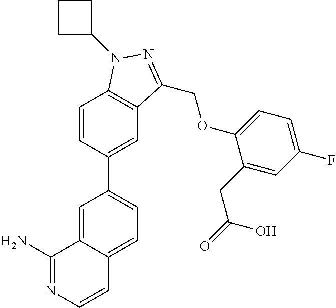 [SMILES: Nc1nccc2ccc(-c3ccc4c(c3)c(COc3ccc(F)cc3CC(=O)O)nn4C3CCC3)cc12]

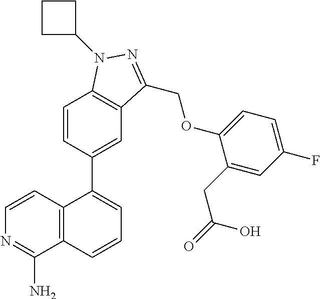 [SMILES: Nc1nccc2c(-c3ccc4c(c3)c(COc3ccc(F)cc3CC(=O)O)nn4C3CCC3)cccc12]

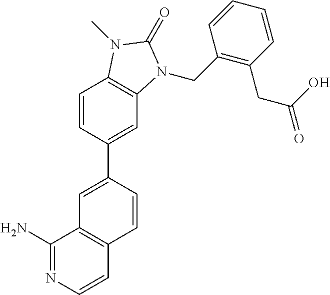 [SMILES: Cn1c(=O)n(Cc2ccccc2CC(=O)O)c2cc(-c3ccc4ccnc(N)c4c3)ccc21]

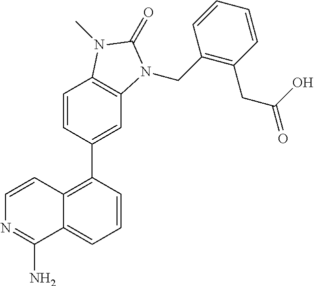 [SMILES: Cn1c(=O)n(Cc2ccccc2CC(=O)O)c2cc(-c3cccc4c(N)nccc34)ccc21]

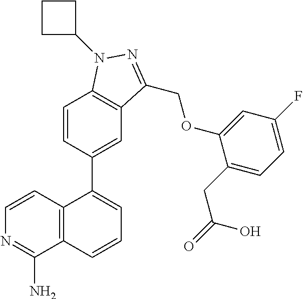 [SMILES: Nc1nccc2c(-c3ccc4c(c3)c(COc3cc(F)ccc3CC(=O)O)nn4C3CCC3)cccc12]

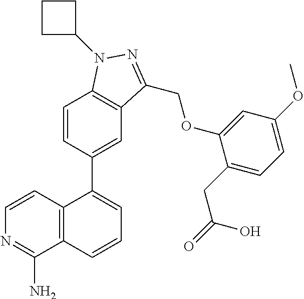 [SMILES: COc1ccc(CC(=O)O)c(OCc2nn(C3CCC3)c3ccc(-c4cccc5c(N)nccc45)cc23)c1]

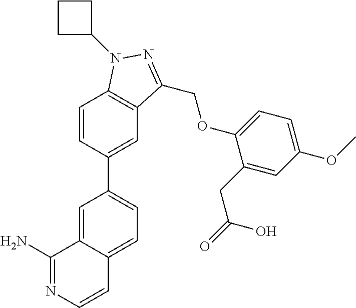 [SMILES: COc1ccc(OCc2nn(C3CCC3)c3ccc(-c4ccc5ccnc(N)c5c4)cc23)c(CC(=O)O)c1]

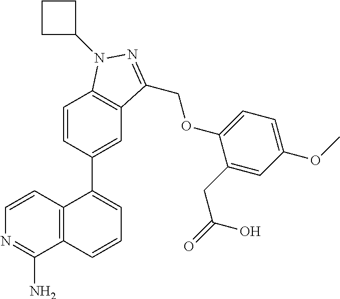 [SMILES: COc1ccc(OCc2nn(C3CCC3)c3ccc(-c4cccc5c(N)nccc45)cc23)c(CC(=O)O)c1]

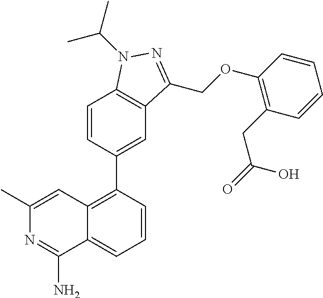 [SMILES: Cc1cc2c(-c3ccc4c(c3)c(COc3ccccc3CC(=O)O)nn4C(C)C)cccc2c(N)n1]

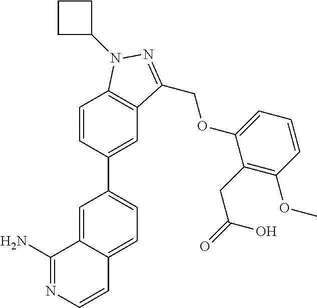 [SMILES: COc1cccc(OCc2nn(C3CCC3)c3ccc(-c4ccc5ccnc(N)c5c4)cc23)c1CC(=O)O]

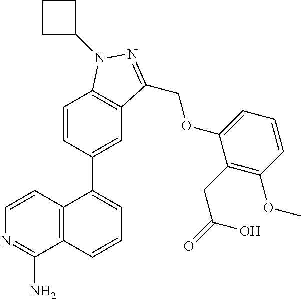 [SMILES: COc1cccc(OCc2nn(C3CCC3)c3ccc(-c4cccc5c(N)nccc45)cc23)c1CC(=O)O]

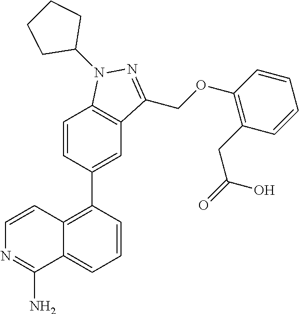 [SMILES: Nc1nccc2c(-c3ccc4c(c3)c(COc3ccccc3CC(=O)O)nn4C3CCCC3)cccc12]

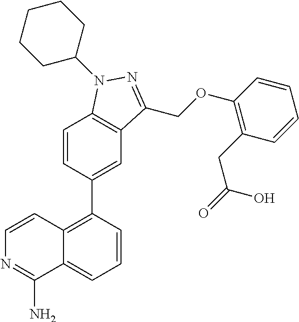 [SMILES: Nc1nccc2c(-c3ccc4c(c3)c(COc3ccccc3CC(=O)O)nn4C3CCCCC3)cccc12]

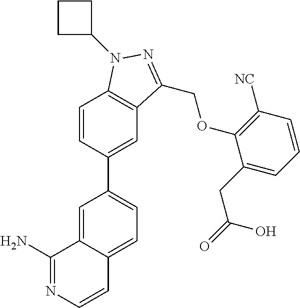 [SMILES: N#Cc1cccc(CC(=O)O)c1OCc1nn(C2CCC2)c2ccc(-c3ccc4ccnc(N)c4c3)cc12]

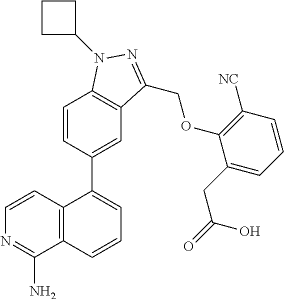 [SMILES: N#Cc1cccc(CC(=O)O)c1OCc1nn(C2CCC2)c2ccc(-c3cccc4c(N)nccc34)cc12]

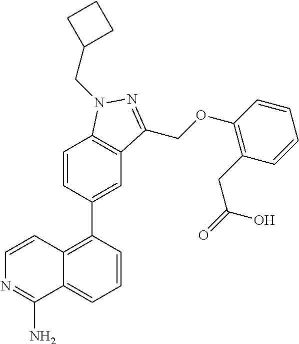 [SMILES: Nc1nccc2c(-c3ccc4c(c3)c(COc3ccccc3CC(=O)O)nn4CC3CCC3)cccc12]

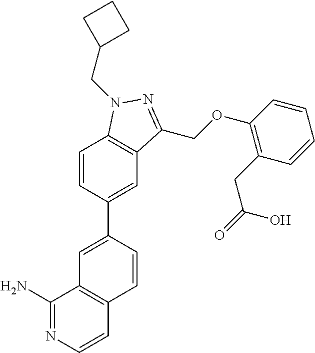 [SMILES: Nc1nccc2ccc(-c3ccc4c(c3)c(COc3ccccc3CC(=O)O)nn4CC3CCC3)cc12]

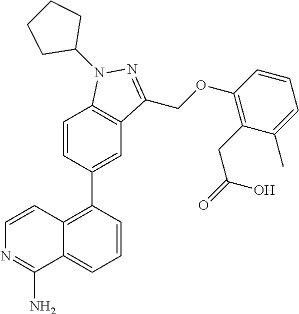 [SMILES: Cc1cccc(OCc2nn(C3CCCC3)c3ccc(-c4cccc5c(N)nccc45)cc23)c1CC(=O)O]

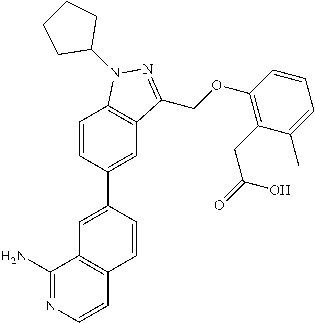 [SMILES: Cc1cccc(OCc2nn(C3CCCC3)c3ccc(-c4ccc5ccnc(N)c5c4)cc23)c1CC(=O)O]

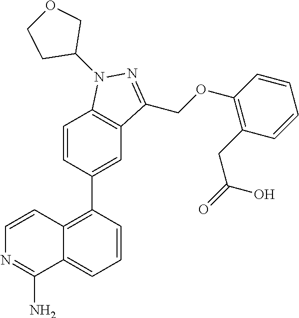 [SMILES: Nc1nccc2c(-c3ccc4c(c3)c(COc3ccccc3CC(=O)O)nn4C3CCOC3)cccc12]

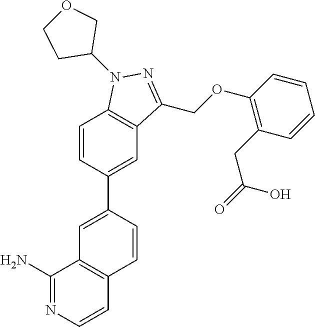 [SMILES: Nc1nccc2ccc(-c3ccc4c(c3)c(COc3ccccc3CC(=O)O)nn4C3CCOC3)cc12]

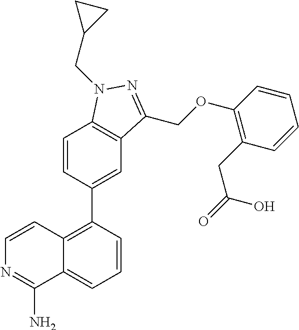 [SMILES: Nc1nccc2c(-c3ccc4c(c3)c(COc3ccccc3CC(=O)O)nn4CC3CC3)cccc12]

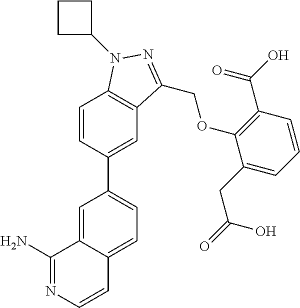 [SMILES: Nc1nccc2ccc(-c3ccc4c(c3)c(COc3c(CC(=O)O)cccc3C(=O)O)nn4C3CCC3)cc12]

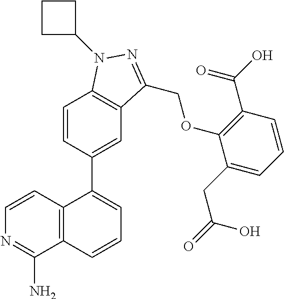 [SMILES: Nc1nccc2c(-c3ccc4c(c3)c(COc3c(CC(=O)O)cccc3C(=O)O)nn4C3CCC3)cccc12]

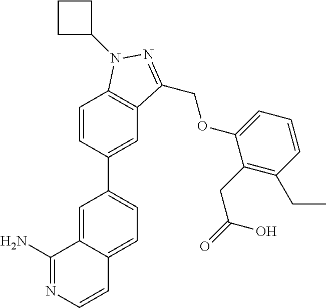 [SMILES: CCc1cccc(OCc2nn(C3CCC3)c3ccc(-c4ccc5ccnc(N)c5c4)cc23)c1CC(=O)O]

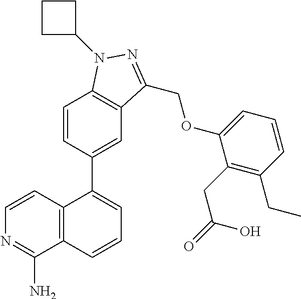 [SMILES: CCc1cccc(OCc2nn(C3CCC3)c3ccc(-c4cccc5c(N)nccc45)cc23)c1CC(=O)O]

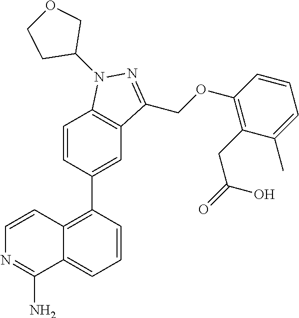 [SMILES: Cc1cccc(OCc2nn(C3CCOC3)c3ccc(-c4cccc5c(N)nccc45)cc23)c1CC(=O)O]

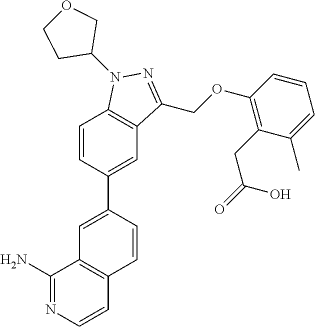 [SMILES: Cc1cccc(OCc2nn(C3CCOC3)c3ccc(-c4ccc5ccnc(N)c5c4)cc23)c1CC(=O)O]

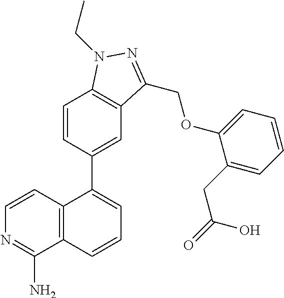 [SMILES: CCn1nc(COc2ccccc2CC(=O)O)c2cc(-c3cccc4c(N)nccc34)ccc21]

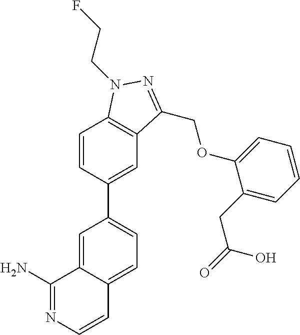 [SMILES: Nc1nccc2ccc(-c3ccc4c(c3)c(COc3ccccc3CC(=O)O)nn4CCF)cc12]

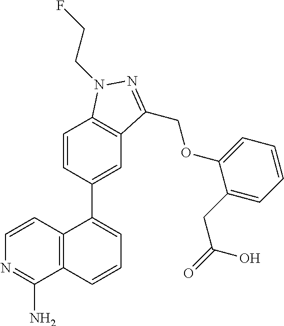 [SMILES: Nc1nccc2c(-c3ccc4c(c3)c(COc3ccccc3CC(=O)O)nn4CCF)cccc12]

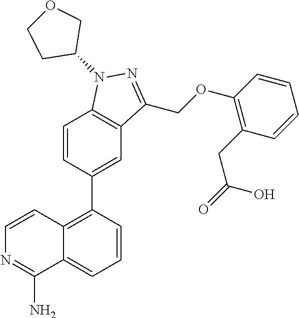 [SMILES: Nc1nccc2c(-c3ccc4c(c3)c(COc3ccccc3CC(=O)O)nn4[C@@H]3CCOC3)cccc12]

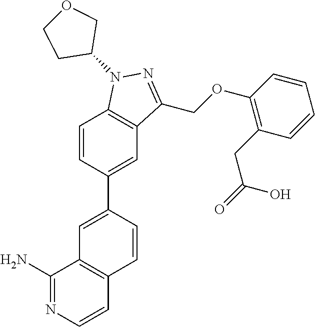 [SMILES: Nc1nccc2ccc(-c3ccc4c(c3)c(COc3ccccc3CC(=O)O)nn4[C@@H]3CCOC3)cc12]

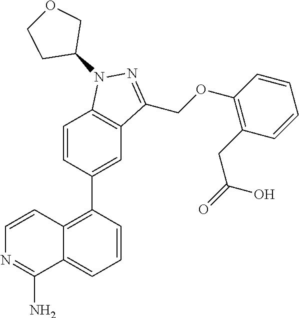 [SMILES: Nc1nccc2c(-c3ccc4c(c3)c(COc3ccccc3CC(=O)O)nn4[C@H]3CCOC3)cccc12]

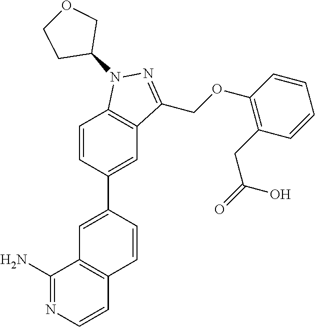 [SMILES: Nc1nccc2ccc(-c3ccc4c(c3)c(COc3ccccc3CC(=O)O)nn4[C@H]3CCOC3)cc12]

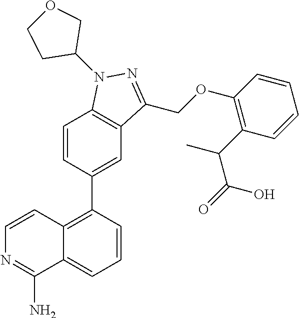 [SMILES: CC(C(=O)O)c1ccccc1OCc1nn(C2CCOC2)c2ccc(-c3cccc4c(N)nccc34)cc12]

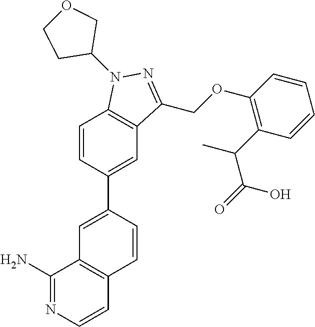 [SMILES: CC(C(=O)O)c1ccccc1OCc1nn(C2CCOC2)c2ccc(-c3ccc4ccnc(N)c4c3)cc12]

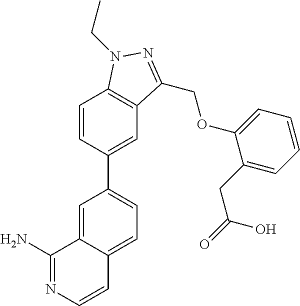 [SMILES: CCn1nc(COc2ccccc2CC(=O)O)c2cc(-c3ccc4ccnc(N)c4c3)ccc21]

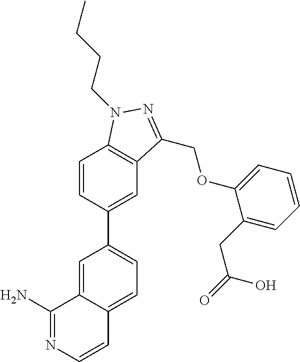 [SMILES: CCCCn1nc(COc2ccccc2CC(=O)O)c2cc(-c3ccc4ccnc(N)c4c3)ccc21]

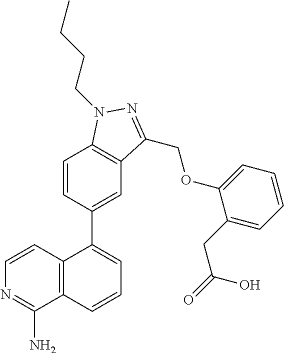 [SMILES: CCCCn1nc(COc2ccccc2CC(=O)O)c2cc(-c3cccc4c(N)nccc34)ccc21]

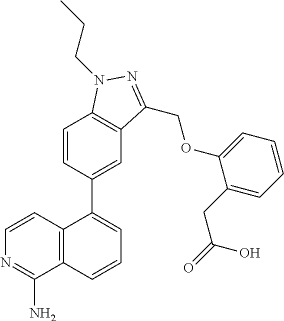 [SMILES: CCCn1nc(COc2ccccc2CC(=O)O)c2cc(-c3cccc4c(N)nccc34)ccc21]

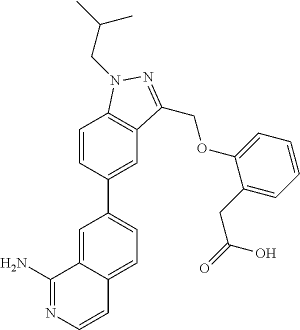 [SMILES: CC(C)Cn1nc(COc2ccccc2CC(=O)O)c2cc(-c3ccc4ccnc(N)c4c3)ccc21]

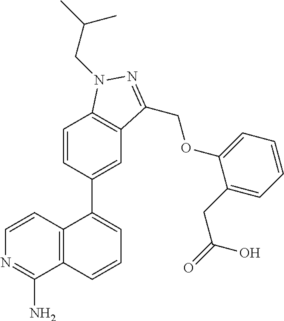 [SMILES: CC(C)Cn1nc(COc2ccccc2CC(=O)O)c2cc(-c3cccc4c(N)nccc34)ccc21]

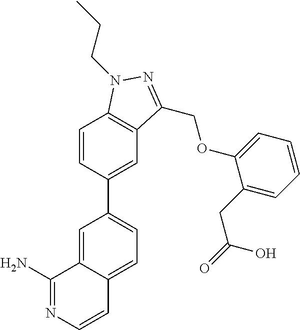 [SMILES: CCCn1nc(COc2ccccc2CC(=O)O)c2cc(-c3ccc4ccnc(N)c4c3)ccc21]

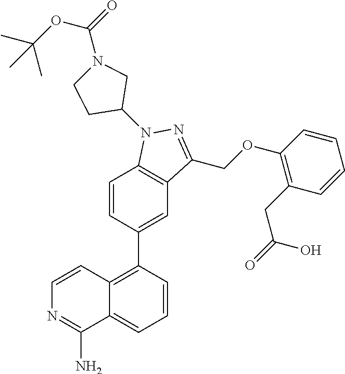 [SMILES: CC(C)(C)OC(=O)N1CCC(n2nc(COc3ccccc3CC(=O)O)c3cc(-c4cccc5c(N)nccc45)ccc32)C1]

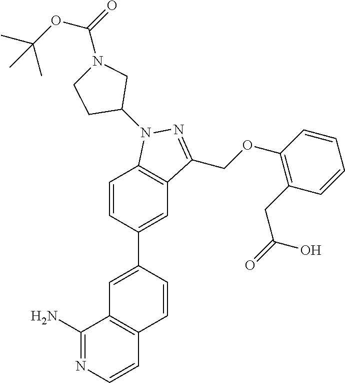 [SMILES: CC(C)(C)OC(=O)N1CCC(n2nc(COc3ccccc3CC(=O)O)c3cc(-c4ccc5ccnc(N)c5c4)ccc32)C1]

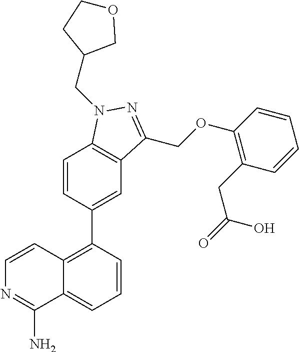 [SMILES: Nc1nccc2c(-c3ccc4c(c3)c(COc3ccccc3CC(=O)O)nn4CC3CCOC3)cccc12]

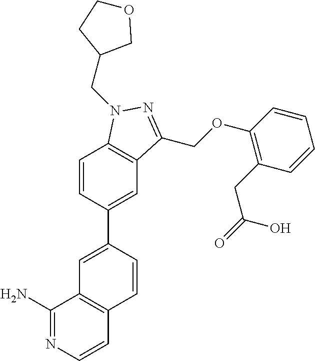 [SMILES: Nc1nccc2ccc(-c3ccc4c(c3)c(COc3ccccc3CC(=O)O)nn4CC3CCOC3)cc12]

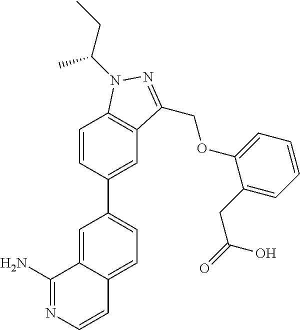 [SMILES: CC[C@@H](C)n1nc(COc2ccccc2CC(=O)O)c2cc(-c3ccc4ccnc(N)c4c3)ccc21]

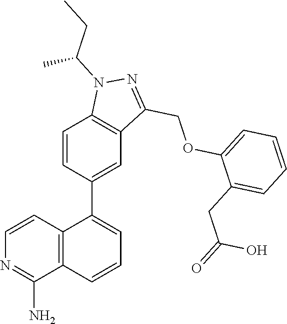 [SMILES: CC[C@@H](C)n1nc(COc2ccccc2CC(=O)O)c2cc(-c3cccc4c(N)nccc34)ccc21]

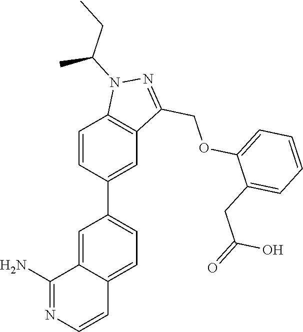 [SMILES: CC[C@H](C)n1nc(COc2ccccc2CC(=O)O)c2cc(-c3ccc4ccnc(N)c4c3)ccc21]

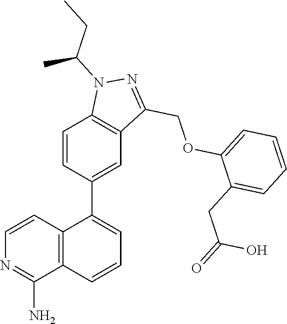 [SMILES: CC[C@H](C)n1nc(COc2ccccc2CC(=O)O)c2cc(-c3cccc4c(N)nccc34)ccc21]

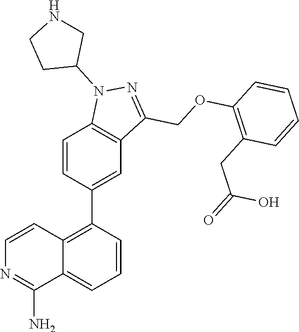 [SMILES: Nc1nccc2c(-c3ccc4c(c3)c(COc3ccccc3CC(=O)O)nn4C3CCNC3)cccc12]

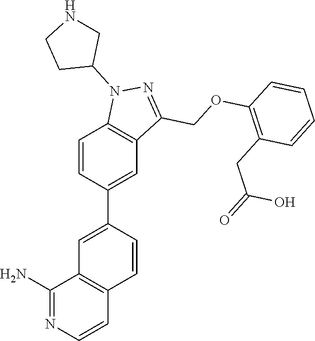 [SMILES: Nc1nccc2ccc(-c3ccc4c(c3)c(COc3ccccc3CC(=O)O)nn4C3CCNC3)cc12]

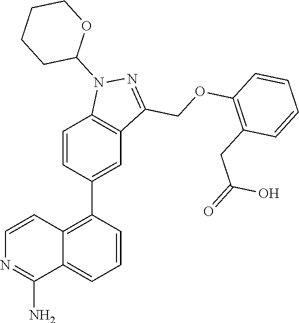 [SMILES: Nc1nccc2c(-c3ccc4c(c3)c(COc3ccccc3CC(=O)O)nn4C3CCCCO3)cccc12]

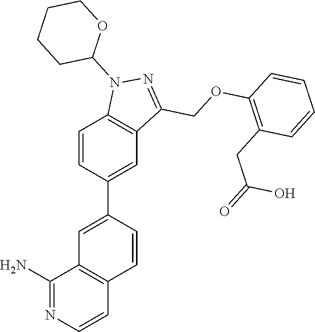 [SMILES: Nc1nccc2ccc(-c3ccc4c(c3)c(COc3ccccc3CC(=O)O)nn4C3CCCCO3)cc12]

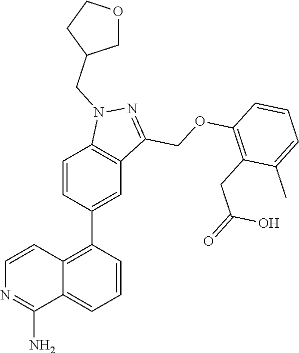 [SMILES: Cc1cccc(OCc2nn(CC3CCOC3)c3ccc(-c4cccc5c(N)nccc45)cc23)c1CC(=O)O]

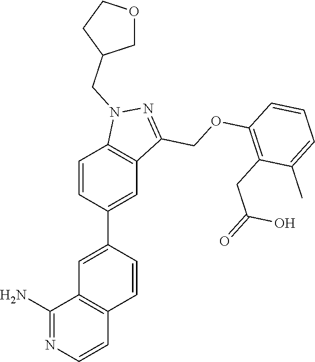 [SMILES: Cc1cccc(OCc2nn(CC3CCOC3)c3ccc(-c4ccc5ccnc(N)c5c4)cc23)c1CC(=O)O]

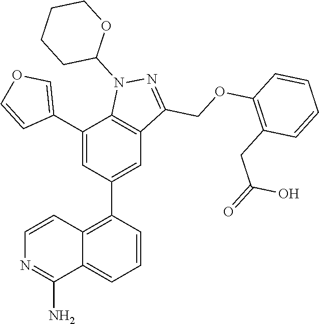 [SMILES: Nc1nccc2c(-c3cc(-c4ccoc4)c4c(c3)c(COc3ccccc3CC(=O)O)nn4C3CCCCO3)cccc12]

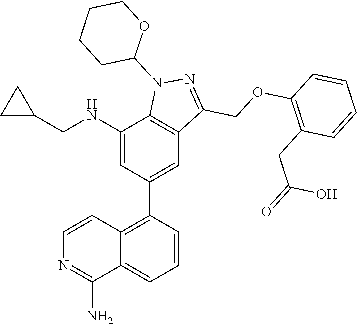 [SMILES: Nc1nccc2c(-c3cc(NCC4CC4)c4c(c3)c(COc3ccccc3CC(=O)O)nn4C3CCCCO3)cccc12]

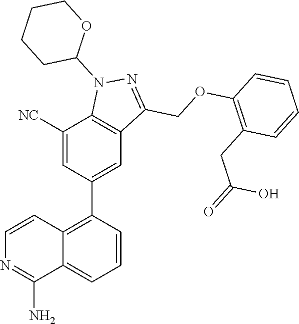 [SMILES: N#Cc1cc(-c2cccc3c(N)nccc23)cc2c(COc3ccccc3CC(=O)O)nn(C3CCCCO3)c12]

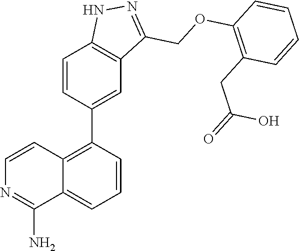 [SMILES: Nc1nccc2c(-c3ccc4[nH]nc(COc5ccccc5CC(=O)O)c4c3)cccc12]

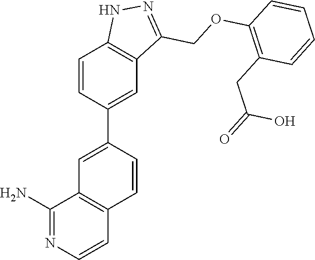 [SMILES: Nc1nccc2ccc(-c3ccc4[nH]nc(COc5ccccc5CC(=O)O)c4c3)cc12]

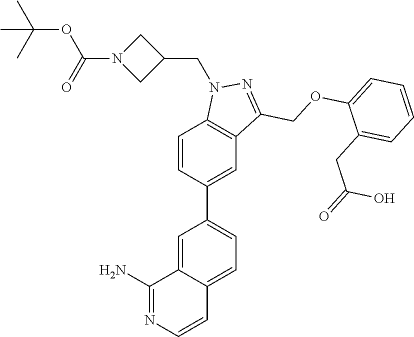 [SMILES: CC(C)(C)OC(=O)N1CC(Cn2nc(COc3ccccc3CC(=O)O)c3cc(-c4ccc5ccnc(N)c5c4)ccc32)C1]

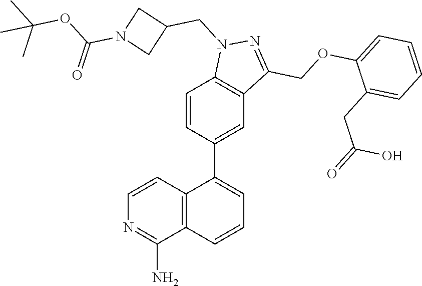 [SMILES: CC(C)(C)OC(=O)N1CC(Cn2nc(COc3ccccc3CC(=O)O)c3cc(-c4cccc5c(N)nccc45)ccc32)C1]

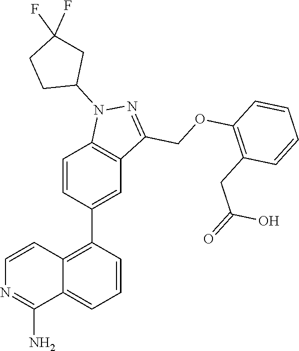 [SMILES: Nc1nccc2c(-c3ccc4c(c3)c(COc3ccccc3CC(=O)O)nn4C3CCC(F)(F)C3)cccc12]

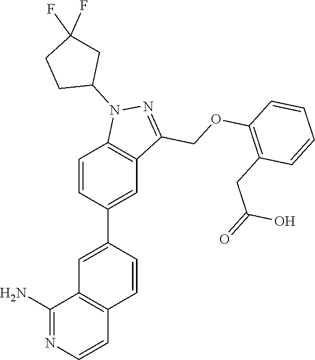 [SMILES: Nc1nccc2ccc(-c3ccc4c(c3)c(COc3ccccc3CC(=O)O)nn4C3CCC(F)(F)C3)cc12]

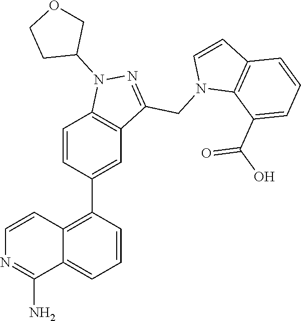 [SMILES: Nc1nccc2c(-c3ccc4c(c3)c(Cn3ccc5cccc(C(=O)O)c53)nn4C3CCOC3)cccc12]

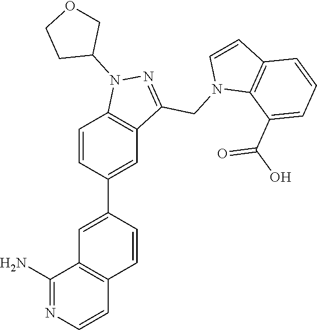 [SMILES: Nc1nccc2ccc(-c3ccc4c(c3)c(Cn3ccc5cccc(C(=O)O)c53)nn4C3CCOC3)cc12]

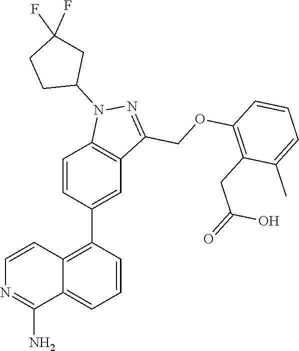 [SMILES: Cc1cccc(OCc2nn(C3CCC(F)(F)C3)c3ccc(-c4cccc5c(N)nccc45)cc23)c1CC(=O)O]

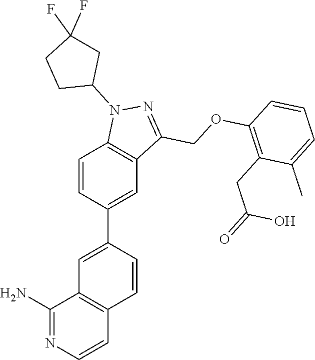 [SMILES: Cc1cccc(OCc2nn(C3CCC(F)(F)C3)c3ccc(-c4ccc5ccnc(N)c5c4)cc23)c1CC(=O)O]

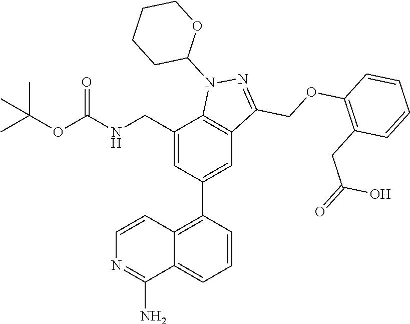 [SMILES: CC(C)(C)OC(=O)NCc1cc(-c2cccc3c(N)nccc23)cc2c(COc3ccccc3CC(=O)O)nn(C3CCCCO3)c12]

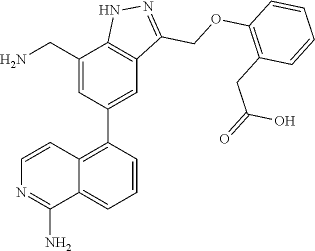 [SMILES: NCc1cc(-c2cccc3c(N)nccc23)cc2c(COc3ccccc3CC(=O)O)n[nH]c12]

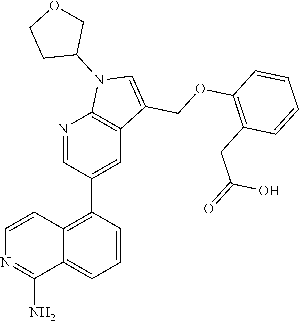 [SMILES: Nc1nccc2c(-c3cnc4c(c3)c(COc3ccccc3CC(=O)O)cn4C3CCOC3)cccc12]

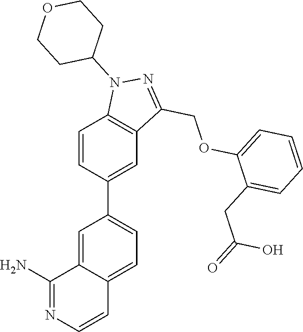 [SMILES: Nc1nccc2ccc(-c3ccc4c(c3)c(COc3ccccc3CC(=O)O)nn4C3CCOCC3)cc12]

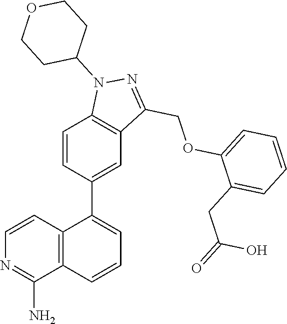 [SMILES: Nc1nccc2c(-c3ccc4c(c3)c(COc3ccccc3CC(=O)O)nn4C3CCOCC3)cccc12]

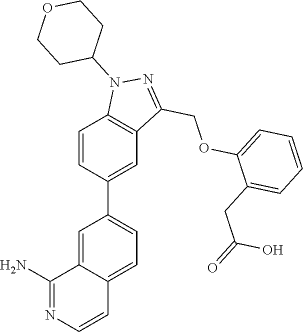 [SMILES: Nc1nccc2ccc(-c3ccc4c(c3)c(COc3ccccc3CC(=O)O)nn4C3CCOCC3)cc12]

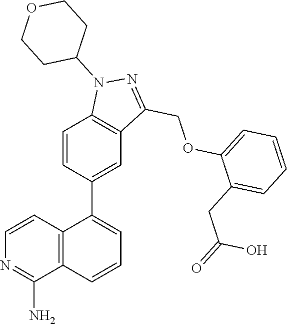 [SMILES: Nc1nccc2c(-c3ccc4c(c3)c(COc3ccccc3CC(=O)O)nn4C3CCOCC3)cccc12]

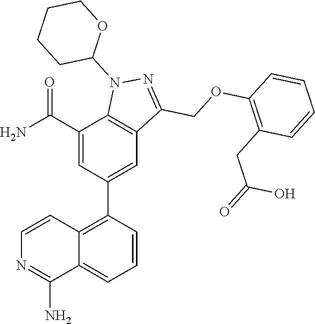 [SMILES: NC(=O)c1cc(-c2cccc3c(N)nccc23)cc2c(COc3ccccc3CC(=O)O)nn(C3CCCCO3)c12]

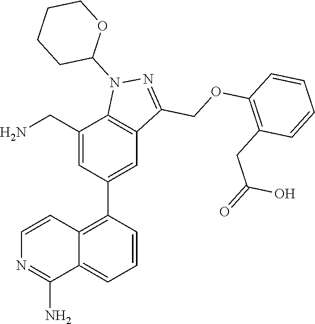 [SMILES: NCc1cc(-c2cccc3c(N)nccc23)cc2c(COc3ccccc3CC(=O)O)nn(C3CCCCO3)c12]

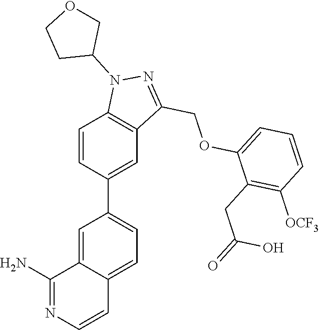 [SMILES: Nc1nccc2ccc(-c3ccc4c(c3)c(COc3cccc(OC(F)(F)F)c3CC(=O)O)nn4C3CCOC3)cc12]

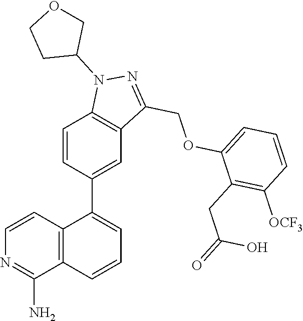 [SMILES: Nc1nccc2c(-c3ccc4c(c3)c(COc3cccc(OC(F)(F)F)c3CC(=O)O)nn4C3CCOC3)cccc12]

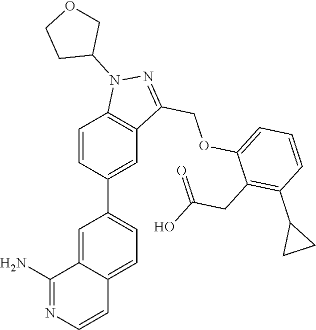 [SMILES: Nc1nccc2ccc(-c3ccc4c(c3)c(COc3cccc(C5CC5)c3CC(=O)O)nn4C3CCOC3)cc12]

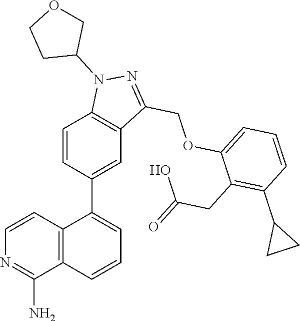 [SMILES: Nc1nccc2c(-c3ccc4c(c3)c(COc3cccc(C5CC5)c3CC(=O)O)nn4C3CCOC3)cccc12]

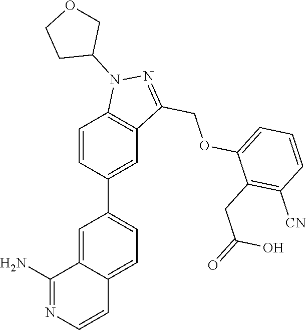 [SMILES: N#Cc1cccc(OCc2nn(C3CCOC3)c3ccc(-c4ccc5ccnc(N)c5c4)cc23)c1CC(=O)O]

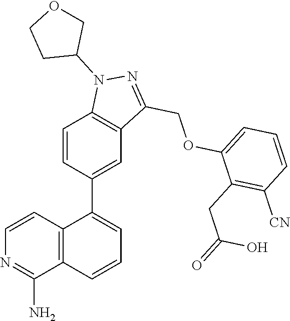 [SMILES: N#Cc1cccc(OCc2nn(C3CCOC3)c3ccc(-c4cccc5c(N)nccc45)cc23)c1CC(=O)O]

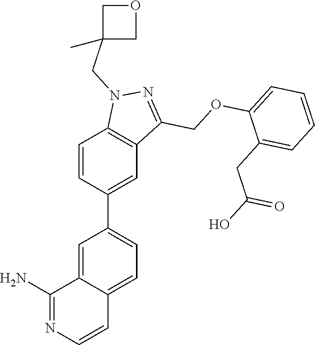 [SMILES: CC1(Cn2nc(COc3ccccc3CC(=O)O)c3cc(-c4ccc5ccnc(N)c5c4)ccc32)COC1]

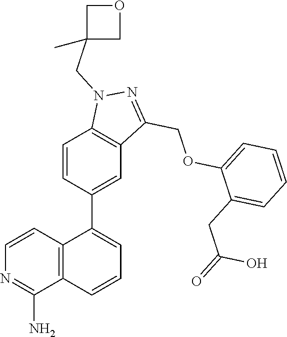 [SMILES: CC1(Cn2nc(COc3ccccc3CC(=O)O)c3cc(-c4cccc5c(N)nccc45)ccc32)COC1]

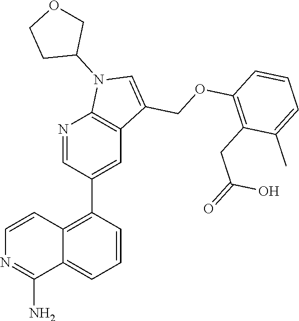 [SMILES: Cc1cccc(OCc2cn(C3CCOC3)c3ncc(-c4cccc5c(N)nccc45)cc23)c1CC(=O)O]

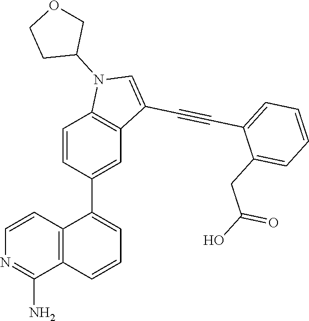 [SMILES: Nc1nccc2c(-c3ccc4c(c3)c(C#Cc3ccccc3CC(=O)O)cn4C3CCOC3)cccc12]

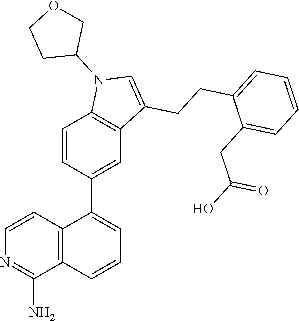 [SMILES: Nc1nccc2c(-c3ccc4c(c3)c(CCc3ccccc3CC(=O)O)cn4C3CCOC3)cccc12]

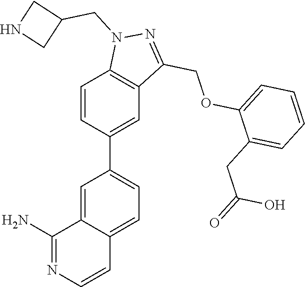 [SMILES: Nc1nccc2ccc(-c3ccc4c(c3)c(COc3ccccc3CC(=O)O)nn4CC3CNC3)cc12]

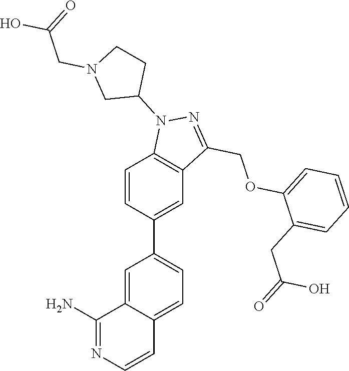 [SMILES: Nc1nccc2ccc(-c3ccc4c(c3)c(COc3ccccc3CC(=O)O)nn4C3CCN(CC(=O)O)C3)cc12]

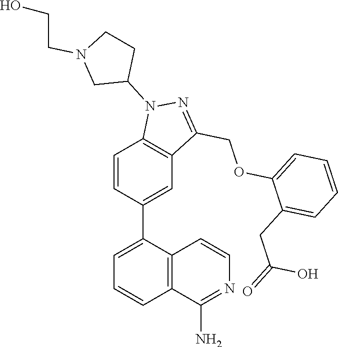 [SMILES: Nc1nccc2c(-c3ccc4c(c3)c(COc3ccccc3CC(=O)O)nn4C3CCN(CCO)C3)cccc12]

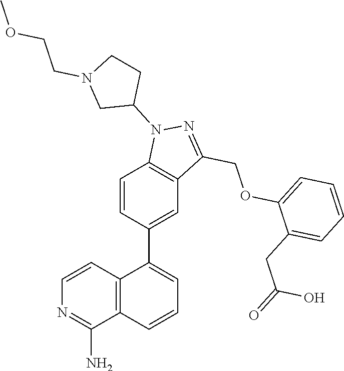 [SMILES: COCCN1CCC(n2nc(COc3ccccc3CC(=O)O)c3cc(-c4cccc5c(N)nccc45)ccc32)C1]

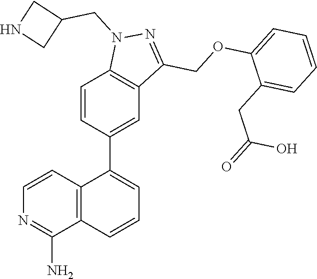 [SMILES: Nc1nccc2c(-c3ccc4c(c3)c(COc3ccccc3CC(=O)O)nn4CC3CNC3)cccc12]

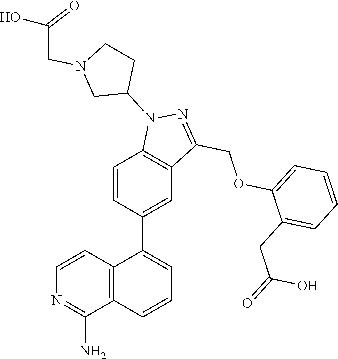 [SMILES: Nc1nccc2c(-c3ccc4c(c3)c(COc3ccccc3CC(=O)O)nn4C3CCN(CC(=O)O)C3)cccc12]

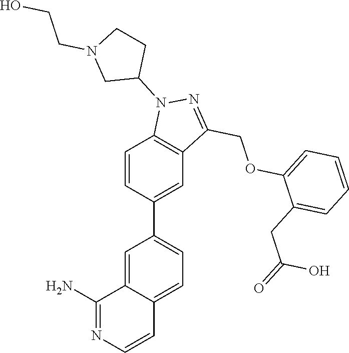 [SMILES: Nc1nccc2ccc(-c3ccc4c(c3)c(COc3ccccc3CC(=O)O)nn4C3CCN(CCO)C3)cc12]

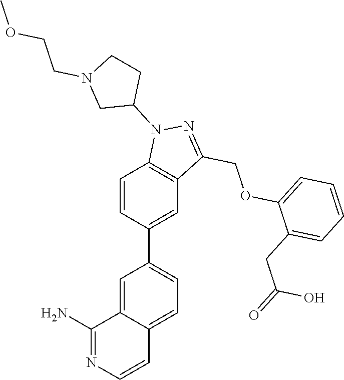 [SMILES: COCCN1CCC(n2nc(COc3ccccc3CC(=O)O)c3cc(-c4ccc5ccnc(N)c5c4)ccc32)C1]

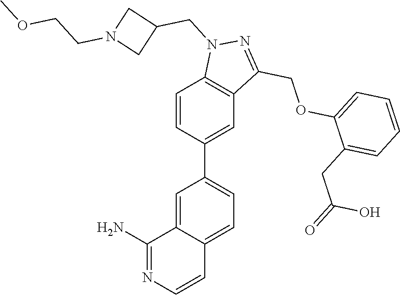 [SMILES: COCCN1CC(Cn2nc(COc3ccccc3CC(=O)O)c3cc(-c4ccc5ccnc(N)c5c4)ccc32)C1]

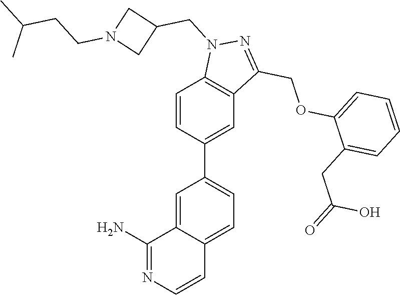 [SMILES: CC(C)CCN1CC(Cn2nc(COc3ccccc3CC(=O)O)c3cc(-c4ccc5ccnc(N)c5c4)ccc32)C1]

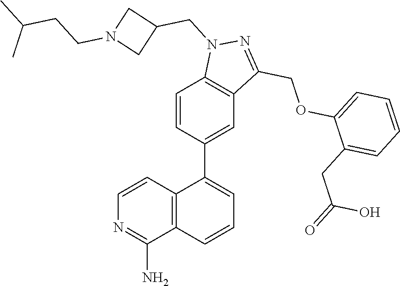 [SMILES: CC(C)CCN1CC(Cn2nc(COc3ccccc3CC(=O)O)c3cc(-c4cccc5c(N)nccc45)ccc32)C1]

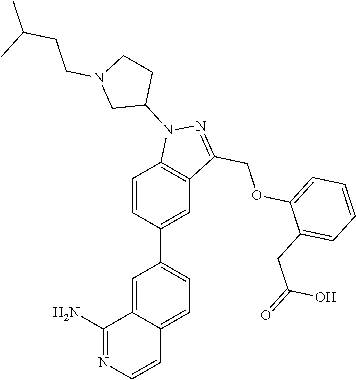 [SMILES: CC(C)CCN1CCC(n2nc(COc3ccccc3CC(=O)O)c3cc(-c4ccc5ccnc(N)c5c4)ccc32)C1]

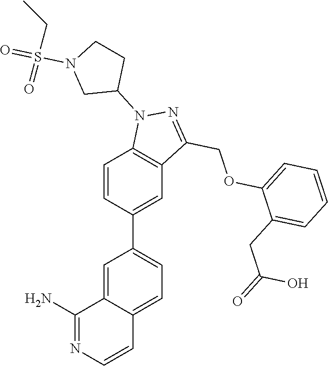 [SMILES: CCS(=O)(=O)N1CCC(n2nc(COc3ccccc3CC(=O)O)c3cc(-c4ccc5ccnc(N)c5c4)ccc32)C1]

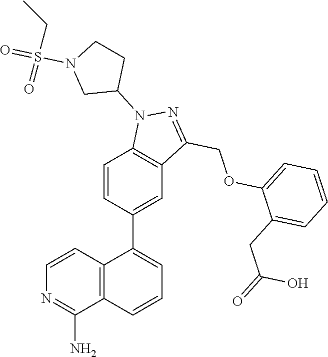 [SMILES: CCS(=O)(=O)N1CCC(n2nc(COc3ccccc3CC(=O)O)c3cc(-c4cccc5c(N)nccc45)ccc32)C1]

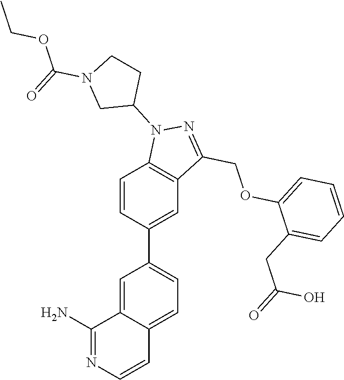 [SMILES: CCOC(=O)N1CCC(n2nc(COc3ccccc3CC(=O)O)c3cc(-c4ccc5ccnc(N)c5c4)ccc32)C1]

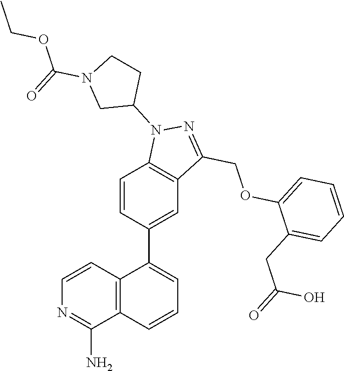 [SMILES: CCOC(=O)N1CCC(n2nc(COc3ccccc3CC(=O)O)c3cc(-c4cccc5c(N)nccc45)ccc32)C1]

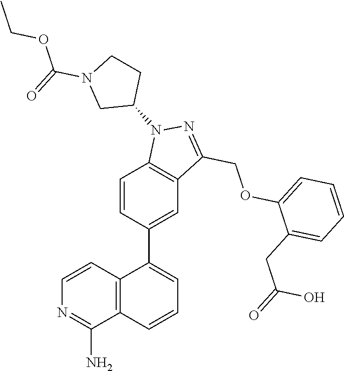 [SMILES: CCOC(=O)N1CC[C@H](n2nc(COc3ccccc3CC(=O)O)c3cc(-c4cccc5c(N)nccc45)ccc32)C1]

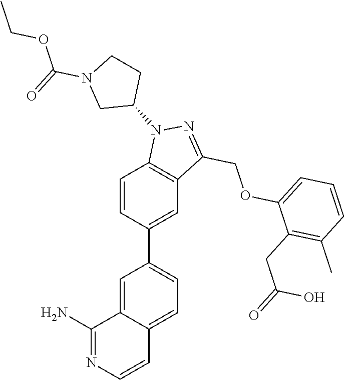 [SMILES: CCOC(=O)N1CC[C@H](n2nc(COc3cccc(C)c3CC(=O)O)c3cc(-c4ccc5ccnc(N)c5c4)ccc32)C1]

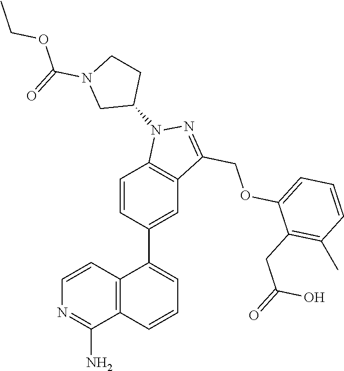 [SMILES: CCOC(=O)N1CC[C@H](n2nc(COc3cccc(C)c3CC(=O)O)c3cc(-c4cccc5c(N)nccc45)ccc32)C1]

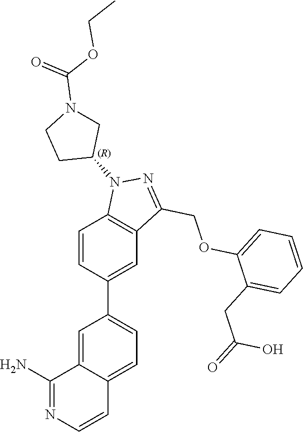

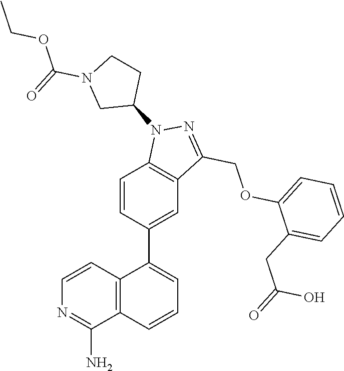 [SMILES: CCOC(=O)N1CC[C@@H](n2nc(COc3ccccc3CC(=O)O)c3cc(-c4cccc5c(N)nccc45)ccc32)C1]

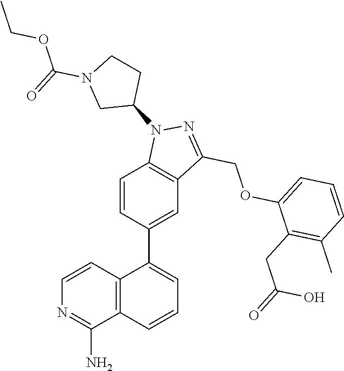 [SMILES: CCOC(=O)N1CC[C@@H](n2nc(COc3cccc(C)c3CC(=O)O)c3cc(-c4cccc5c(N)nccc45)ccc32)C1]

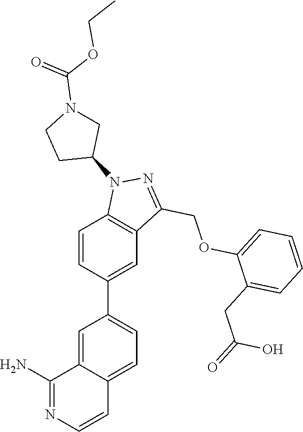 [SMILES: CCOC(=O)N1CC[C@H](n2nc(COc3ccccc3CC(=O)O)c3cc(-c4ccc5ccnc(N)c5c4)ccc32)C1]

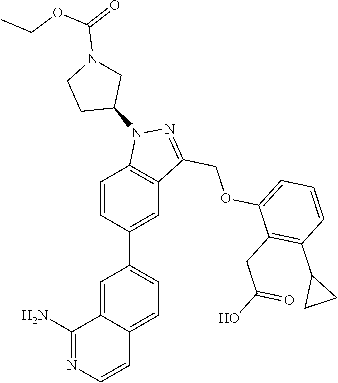 [SMILES: CCOC(=O)N1CC[C@H](n2nc(COc3cccc(C4CC4)c3CC(=O)O)c3cc(-c4ccc5ccnc(N)c5c4)ccc32)C1]

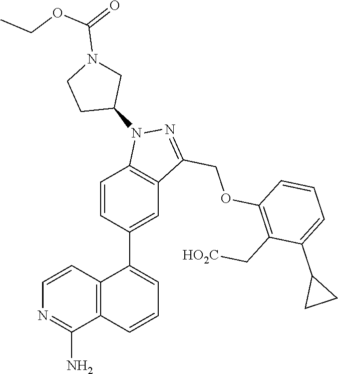 [SMILES: CCOC(=O)N1CC[C@H](n2nc(COc3cccc(C4CC4)c3CC(=O)O)c3cc(-c4cccc5c(N)nccc45)ccc32)C1]

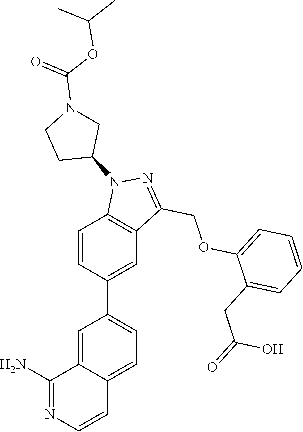 [SMILES: CC(C)OC(=O)N1CC[C@H](n2nc(COc3ccccc3CC(=O)O)c3cc(-c4ccc5ccnc(N)c5c4)ccc32)C1]

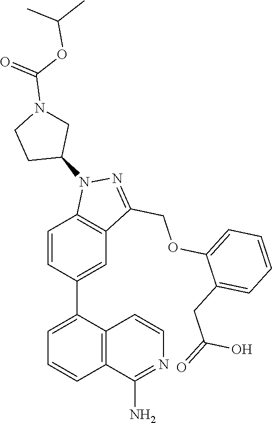 [SMILES: CC(C)OC(=O)N1CC[C@H](n2nc(COc3ccccc3CC(=O)O)c3cc(-c4cccc5c(N)nccc45)ccc32)C1]

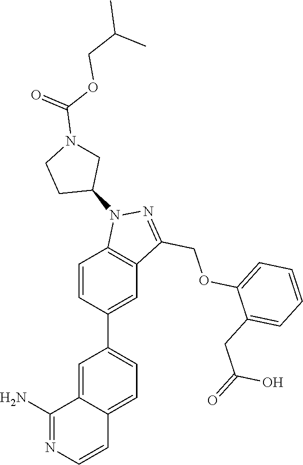 [SMILES: CC(C)COC(=O)N1CC[C@H](n2nc(COc3ccccc3CC(=O)O)c3cc(-c4ccc5ccnc(N)c5c4)ccc32)C1]

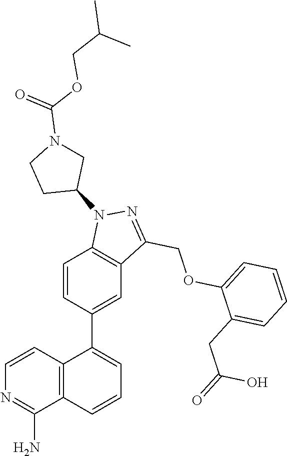 [SMILES: CC(C)COC(=O)N1CC[C@H](n2nc(COc3ccccc3CC(=O)O)c3cc(-c4cccc5c(N)nccc45)ccc32)C1]

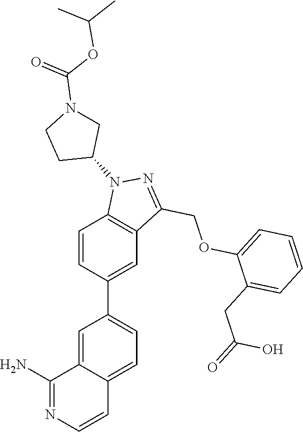 [SMILES: CC(C)OC(=O)N1CC[C@@H](n2nc(COc3ccccc3CC(=O)O)c3cc(-c4ccc5ccnc(N)c5c4)ccc32)C1]

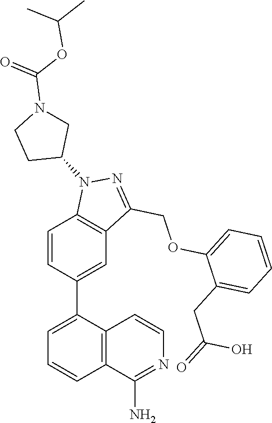 [SMILES: CC(C)OC(=O)N1CC[C@@H](n2nc(COc3ccccc3CC(=O)O)c3cc(-c4cccc5c(N)nccc45)ccc32)C1]

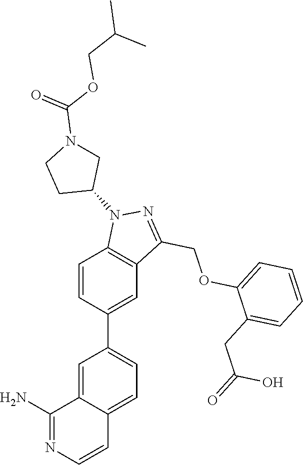 [SMILES: CC(C)COC(=O)N1CC[C@@H](n2nc(COc3ccccc3CC(=O)O)c3cc(-c4ccc5ccnc(N)c5c4)ccc32)C1]

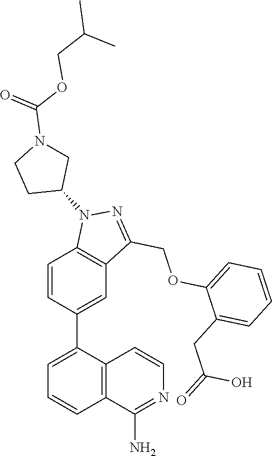 [SMILES: CC(C)COC(=O)N1CC[C@@H](n2nc(COc3ccccc3CC(=O)O)c3cc(-c4cccc5c(N)nccc45)ccc32)C1]

The present invention provides also compounds having the structure of Formula (III), and pharmaceutically acceptable salts thereof:

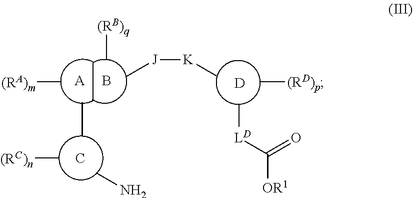

wherein
ring

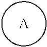

is arylene or heteroarylene;
ring

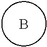

is cycloalkylene, heterocycloalkylene, cycloalkenylene, of heterocycloalkenylene;
ring

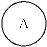

is fused to ring

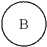

at two and only two adjacent positions;
ring

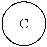

is a bicyclic ring system, where the ring attached to ring

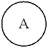

is aryl or heteroaryl;
ring

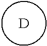

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-. —C(alkyl)₂-, —CH(cycloalkyl)-, or

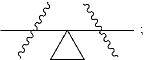 [SMILES: *C1(*)CC1]

K represents a bond. —O—, —NH—, —C(O)—, —CH₂—, —S—, —S(O)—, —SO—, —N(alkyl)-, —CH(alkyl)-, or —CH(cycloalkyl)-:
   wherein at least one of J and K is a bond, —C(O)—, —CH₂—, —CH₂CH₂—, —CH(alkyl)-, or —CH(aryl)₂-;
L^{D} represents —CH₂—, —CH₂CH₂—. —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl). —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl. —NH(heteroarylalkyl), —CH₂O(optionally substituted aryl), —C(O)O(alkyl), —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two adjacent occurrences of R^{B} taken together with the intervening atoms form an aromatic ring; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{D}, independently for each occurrence, represents H, halo, hydroxyl, cyano. —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

In certain embodiments, ring

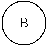

is cycloalkylene or heterocycloalkylene. In certain embodiments, ring

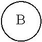

is cycloalkylene. In certain embodiments, ring

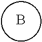

is cycloalkenylene or heterocycloalkenylene. In certain embodiments, ring

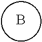

is cycloalkenylene.

In certain embodiments, in the compound having the structure of Formula (III),
ring

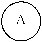

is arylene;
ring

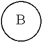

is cycloalkylene or cycloalkenylene;
ring

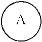

is fused to ring

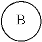

at two and only two adjacent positions;
ring

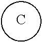

is a bicyclic ring system, where the ring attached to ring

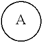

is aryl;
ring

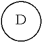

is aryl;
J represents —O—;
K represents a bond;
L^{D} represents —CH₂—;
R^{A}, independently for each occurrence, represents H, halo, alkyl, optionally substituted aryl, optionally substituted heteroaryl, or optionally substituted cycloalkyl;
R^{B}, independently for each occurrence, represents H, or two adjacent occurrences of R^{B} taken together with the intervening atoms form an aromatic ring; or two geminal occurrences of R^{B} taken together with the atom to which they are attached form an optionally substituted spirocycloalkyl or spiroheterocycloalkyl;
R^{C} represents H;
R^{D}, independently for each occurrence, represents H or optionally substituted alkyl;
R¹ represents H.

In certain embodiments, ring

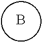

is cycloalkylene. In certain embodiments, ring

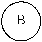

is cycloalkenylene.

In certain embodiments, the compound of Formula (III) has the structure of formula (IIIa):

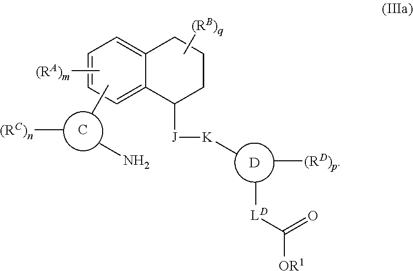

For example, the compound of Formula (III) may have the structure of formula (IIIa-1):

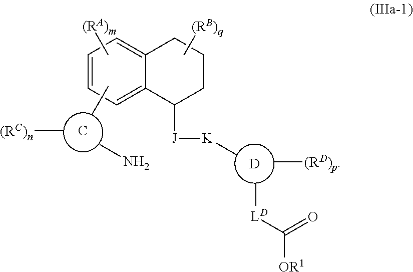

In certain embodiments, the compound of Formula (III) has the structure of formula (IIIb):

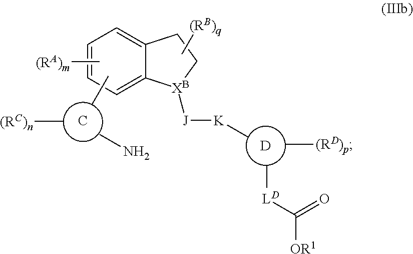

wherein X^{B} represents CH or N; in certain embodiments, X^{B} represents CH.

For example, the compound of Formula (III) may have the structure of formula (IIIb-1):

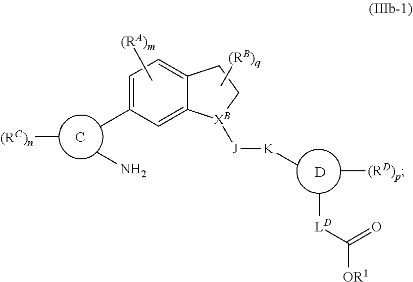

wherein X^{B} represents CH or N; in certain embodiments, X^{B} represents CH.

In certain embodiments, the compound of Formula (III) has the structure of formula (IIIc):

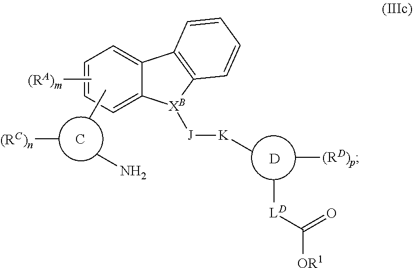

wherein X^{B} represents CH or N; in certain embodiments, X^{B} represents CH.

For example, the compound of Formula (III) may have the structure of formula (IIIc-1):

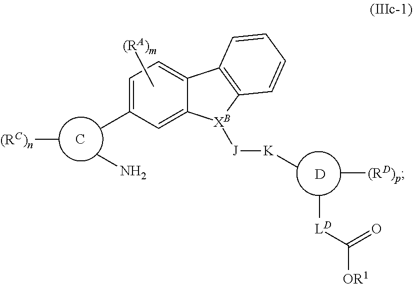

wherein X^{B} represents CH or N.

In certain embodiments, the compound of Formula (III) has the structure of formula (IIId):

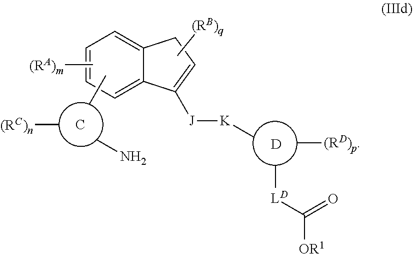

For example, the compound of Formula (III) may have the structure of formula (IIId-1):

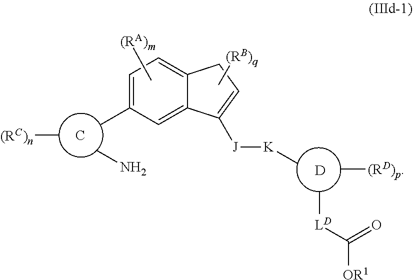

In some embodiments, ring

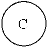

is a bicyclic ring system, where the ring attached to ring

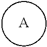

is aryl.

In certain embodiments, represents

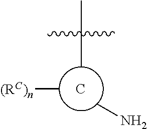

represents

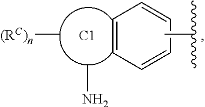

wherein ring

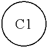

represents heteroaryl. In exemplary embodiments,

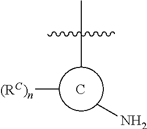

represents

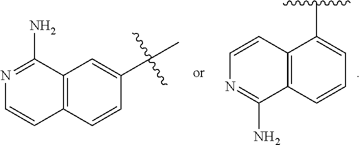 [SMILES: *c1ccc2ccnc(N)c2c1 ; *c1cccc2c(N)nccc12]

In certain embodiments, R^{C} represents H.

In certain embodiments, J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

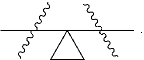 [SMILES: *C1(*)CC1]

In certain embodiments, -J-K- represents —O—.

In any of the foregoing embodiments, ring

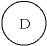

may represent aryl.

In certain embodiments,

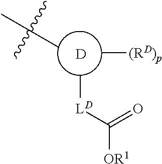

represents

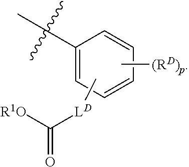

For example,

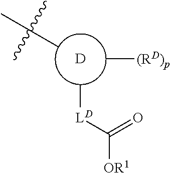

may represent

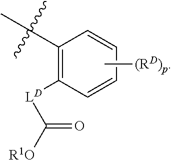

In certain embodiments, ring

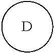

is bicyclic heteroaryl. For example, in some embodiments,

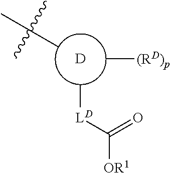

represents

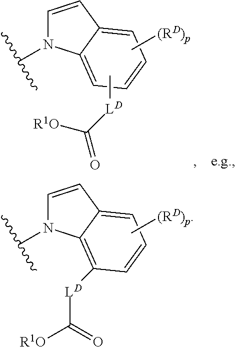

In certain embodiments, m, n, p, and q are each independently 0 or 1.

In certain embodiments, R¹ is H.

In certain embodiments, LF represents —CH₂—.

In certain embodiments, R^{D}, independently for each occurrence, represents H or alkyl.

In certain embodiments, R^{B} represents H.

In certain embodiments, RE represents optionally substituted cycloalkyl or optionally substituted heterocycloalkyl.

In certain embodiments, two geminal occurrences of R^{B} taken together with the atom to which they are attached form an optionally substituted spirocycloalkyl or a spiroheterocycloalkyl. In certain such embodiments, two geminal occurrences of R^{B} taken together with the atom to which they are attached form a substituted spiroheterocycloalkyl.

In certain embodiments. R^{A}, independently for each occurrence, represents H, halo, alkyl aryl, heteroaryl, or cycloalkyl. In some embodiments. R^{A} represents H.

In certain embodiments, the compound of formula (III) is selected from the following table of compounds, and pharmaceutically acceptable salts thereof:

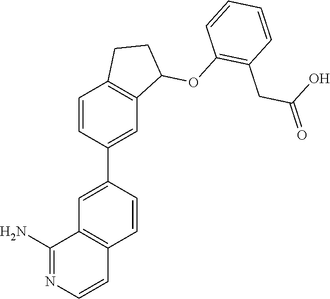 [SMILES: Nc1nccc2ccc(-c3ccc4c(c3)C(Oc3ccccc3CC(=O)O)CC4)cc12]

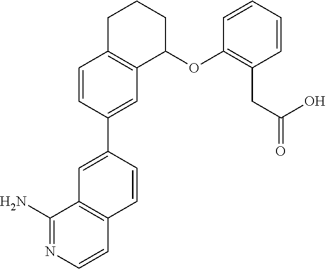 [SMILES: Nc1nccc2ccc(-c3ccc4c(c3)C(Oc3ccccc3CC(=O)O)CCC4)cc12]

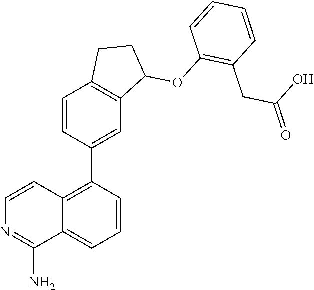 [SMILES: Nc1nccc2c(-c3ccc4c(c3)C(Oc3ccccc3CC(=O)O)CC4)cccc12]

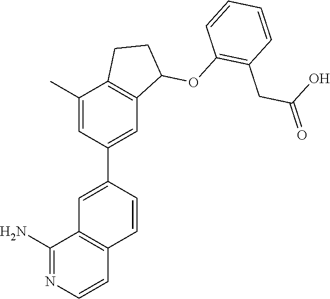 [SMILES: Cc1cc(-c2ccc3ccnc(N)c3c2)cc2c1CCC2Oc1ccccc1CC(=O)O]

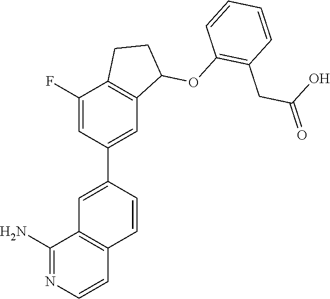 [SMILES: Nc1nccc2ccc(-c3cc(F)c4c(c3)C(Oc3ccccc3CC(=O)O)CC4)cc12]

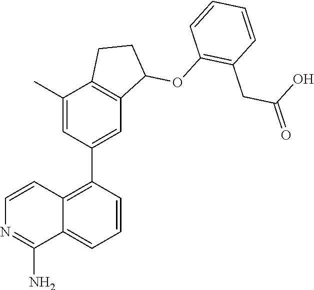 [SMILES: Cc1cc(-c2cccc3c(N)nccc23)cc2c1CCC2Oc1ccccc1CC(=O)O]

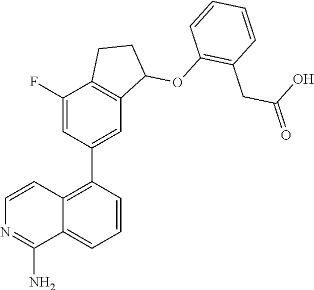 [SMILES: Nc1nccc2c(-c3cc(F)c4c(c3)C(Oc3ccccc3CC(=O)O)CC4)cccc12]

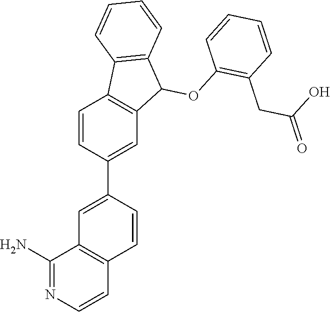 [SMILES: Nc1nccc2ccc(-c3ccc4c(c3)C(Oc3ccccc3CC(=O)O)c3ccccc3-4)cc12]

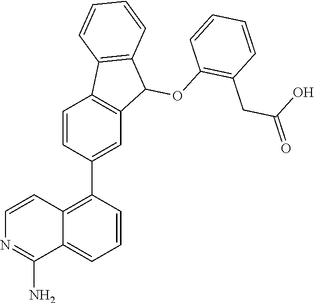 [SMILES: Nc1nccc2c(-c3ccc4c(c3)C(Oc3ccccc3CC(=O)O)c3ccccc3-4)cccc12]

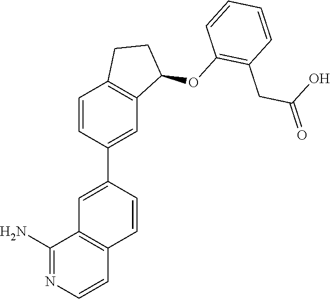 [SMILES: Nc1nccc2ccc(-c3ccc4c(c3)[C@H](Oc3ccccc3CC(=O)O)CC4)cc12]

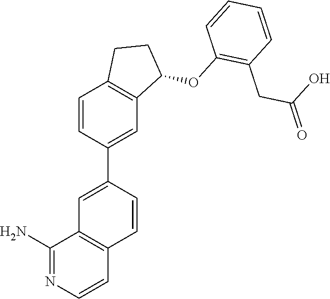 [SMILES: Nc1nccc2ccc(-c3ccc4c(c3)[C@@H](Oc3ccccc3CC(=O)O)CC4)cc12]

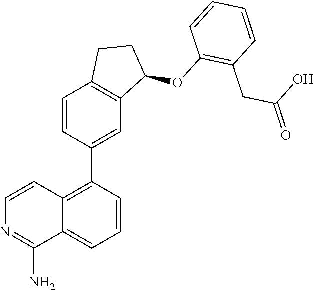 [SMILES: Nc1nccc2c(-c3ccc4c(c3)[C@H](Oc3ccccc3CC(=O)O)CC4)cccc12]

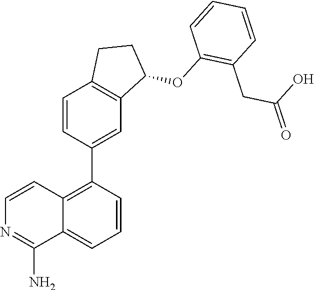 [SMILES: Nc1nccc2c(-c3ccc4c(c3)[C@@H](Oc3ccccc3CC(=O)O)CC4)cccc12]

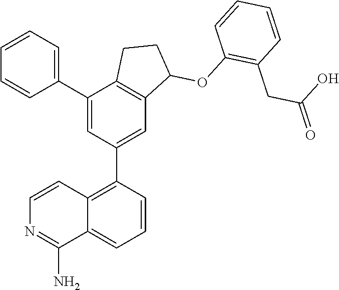 [SMILES: Nc1nccc2c(-c3cc(-c4ccccc4)c4c(c3)C(Oc3ccccc3CC(=O)O)CC4)cccc12]

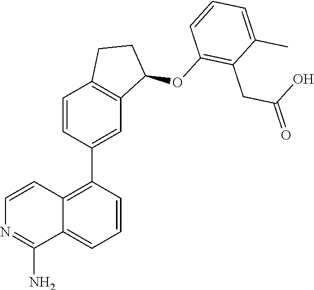 [SMILES: Cc1cccc(O[C@@H]2CCc3ccc(-c4cccc5c(N)nccc45)cc32)c1CC(=O)O]

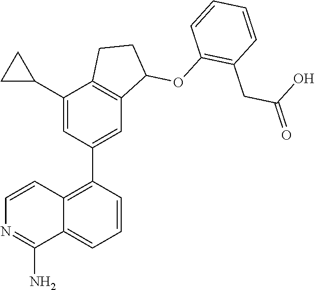 [SMILES: Nc1nccc2c(-c3cc(C4CC4)c4c(c3)C(Oc3ccccc3CC(=O)O)CC4)cccc12]

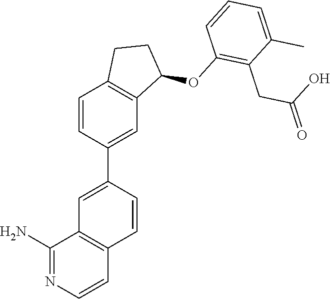 [SMILES: Cc1cccc(O[C@@H]2CCc3ccc(-c4ccc5ccnc(N)c5c4)cc32)c1CC(=O)O]

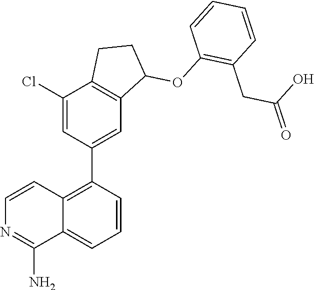 [SMILES: Nc1nccc2c(-c3cc(Cl)c4c(c3)C(Oc3ccccc3CC(=O)O)CC4)cccc12]

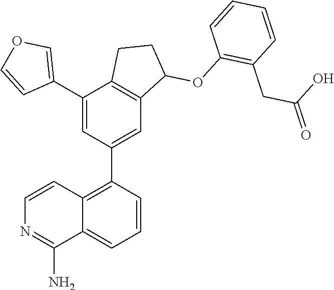 [SMILES: Nc1nccc2c(-c3cc(-c4ccoc4)c4c(c3)C(Oc3ccccc3CC(=O)O)CC4)cccc12]

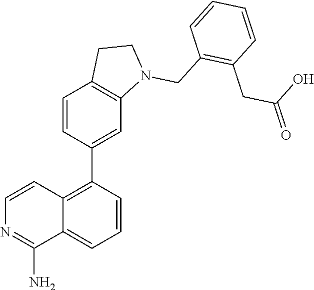 [SMILES: Nc1nccc2c(-c3ccc4c(c3)N(Cc3ccccc3CC(=O)O)CC4)cccc12]

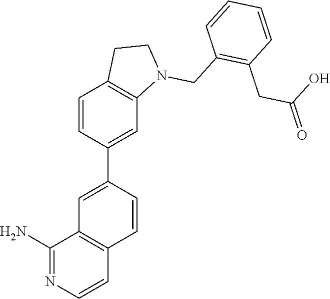 [SMILES: Nc1nccc2ccc(-c3ccc4c(c3)N(Cc3ccccc3CC(=O)O)CC4)cc12]

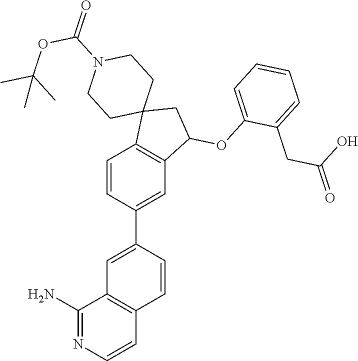 [SMILES: CC(C)(C)OC(=O)N1CCC2(CC1)CC(Oc1ccccc1CC(=O)O)c1cc(-c3ccc4ccnc(N)c4c3)ccc12]

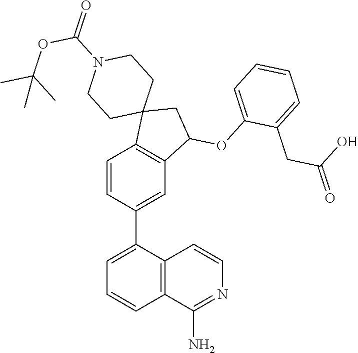 [SMILES: CC(C)(C)OC(=O)N1CCC2(CC1)CC(Oc1ccccc1CC(=O)O)c1cc(-c3cccc4c(N)nccc34)ccc12]

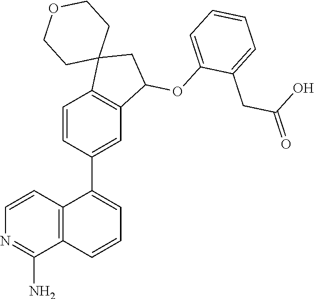 [SMILES: Nc1nccc2c(-c3ccc4c(c3)C(Oc3ccccc3CC(=O)O)CC43CCOCC3)cccc12]

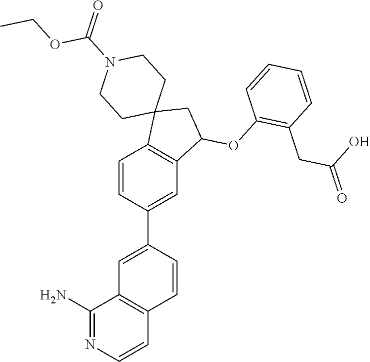 [SMILES: CCOC(=O)N1CCC2(CC1)CC(Oc1ccccc1CC(=O)O)c1cc(-c3ccc4ccnc(N)c4c3)ccc12]

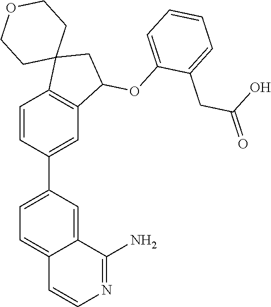 [SMILES: Nc1nccc2ccc(-c3ccc4c(c3)C(Oc3ccccc3CC(=O)O)CC43CCOCC3)cc12]

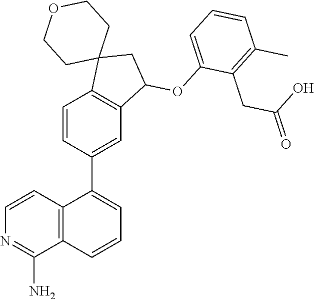 [SMILES: Cc1cccc(OC2CC3(CCOCC3)c3ccc(-c4cccc5c(N)nccc45)cc32)c1CC(=O)O]

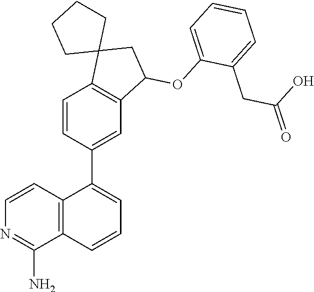 [SMILES: Nc1nccc2c(-c3ccc4c(c3)C(Oc3ccccc3CC(=O)O)CC43CCCC3)cccc12]

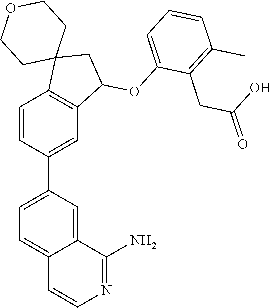 [SMILES: Cc1cccc(OC2CC3(CCOCC3)c3ccc(-c4ccc5ccnc(N)c5c4)cc32)c1CC(=O)O]

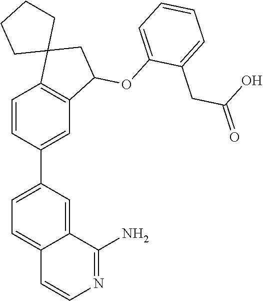 [SMILES: Nc1nccc2ccc(-c3ccc4c(c3)C(Oc3ccccc3CC(=O)O)CC43CCCC3)cc12]

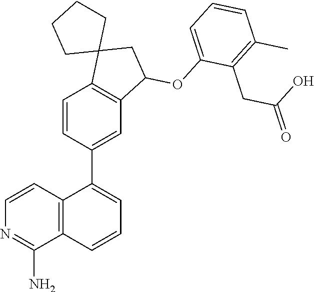 [SMILES: Cc1cccc(OC2CC3(CCCC3)c3ccc(-c4cccc5c(N)nccc45)cc32)c1CC(=O)O]

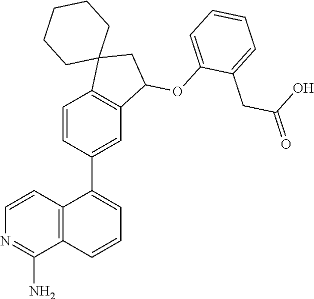 [SMILES: Nc1nccc2c(-c3ccc4c(c3)C(Oc3ccccc3CC(=O)O)CC43CCCCC3)cccc12]

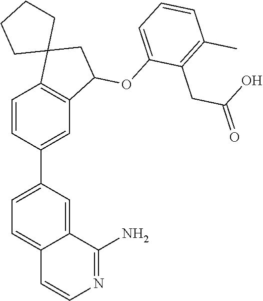 [SMILES: Cc1cccc(OC2CC3(CCCC3)c3ccc(-c4ccc5ccnc(N)c5c4)cc32)c1CC(=O)O]

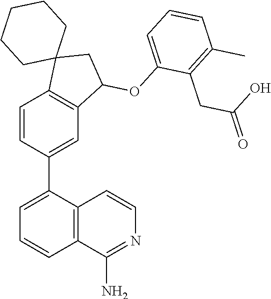 [SMILES: Cc1cccc(OC2CC3(CCCCC3)c3ccc(-c4cccc5c(N)nccc45)cc32)c1CC(=O)O]

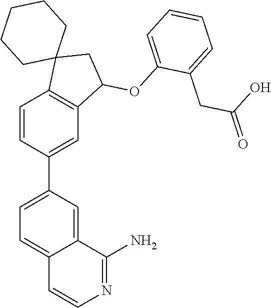 [SMILES: Nc1nccc2ccc(-c3ccc4c(c3)C(Oc3ccccc3CC(=O)O)CC43CCCCC3)cc12]

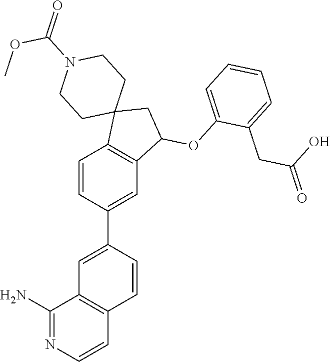 [SMILES: COC(=O)N1CCC2(CC1)CC(Oc1ccccc1CC(=O)O)c1cc(-c3ccc4ccnc(N)c4c3)ccc12]

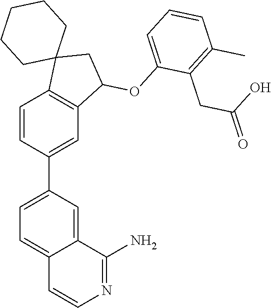 [SMILES: Cc1cccc(OC2CC3(CCCCC3)c3ccc(-c4ccc5ccnc(N)c5c4)cc32)c1CC(=O)O]

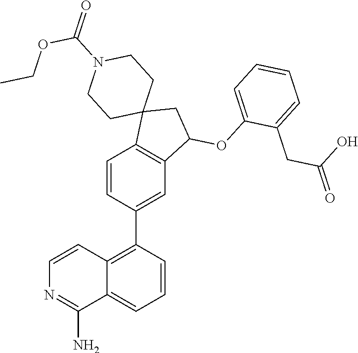 [SMILES: CCOC(=O)N1CCC2(CC1)CC(Oc1ccccc1CC(=O)O)c1cc(-c3cccc4c(N)nccc34)ccc12]

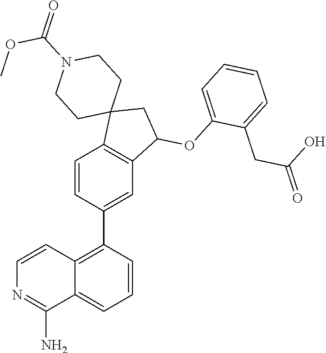 [SMILES: COC(=O)N1CCC2(CC1)CC(Oc1ccccc1CC(=O)O)c1cc(-c3cccc4c(N)nccc34)ccc12]

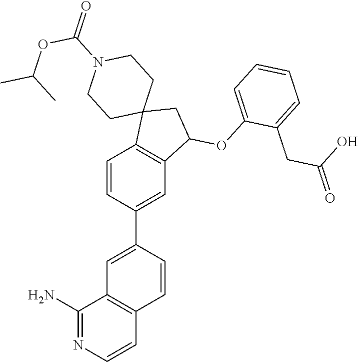 [SMILES: CC(C)OC(=O)N1CCC2(CC1)CC(Oc1ccccc1CC(=O)O)c1cc(-c3ccc4ccnc(N)c4c3)ccc12]

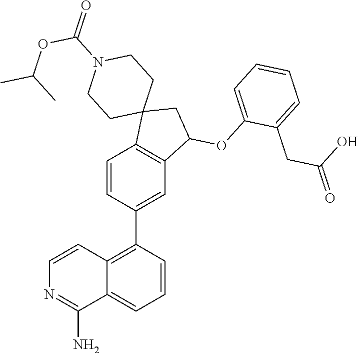 [SMILES: CC(C)OC(=O)N1CCC2(CC1)CC(Oc1ccccc1CC(=O)O)c1cc(-c3cccc4c(N)nccc34)ccc12]

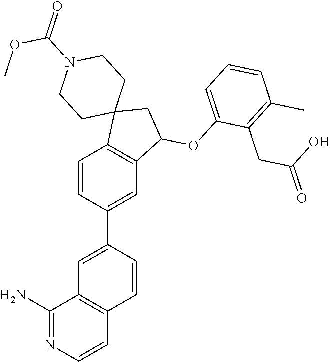 [SMILES: COC(=O)N1CCC2(CC1)CC(Oc1cccc(C)c1CC(=O)O)c1cc(-c3ccc4ccnc(N)c4c3)ccc12]

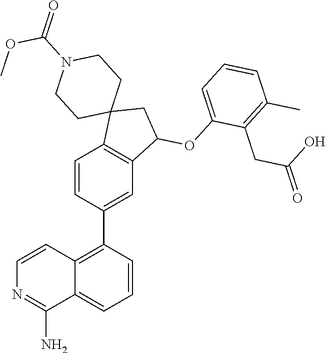 [SMILES: COC(=O)N1CCC2(CC1)CC(Oc1cccc(C)c1CC(=O)O)c1cc(-c3cccc4c(N)nccc34)ccc12]

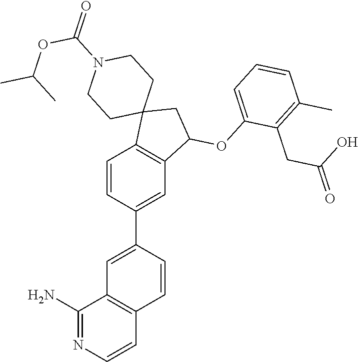 [SMILES: Cc1cccc(OC2CC3(CCN(C(=O)OC(C)C)CC3)c3ccc(-c4ccc5ccnc(N)c5c4)cc32)c1CC(=O)O]

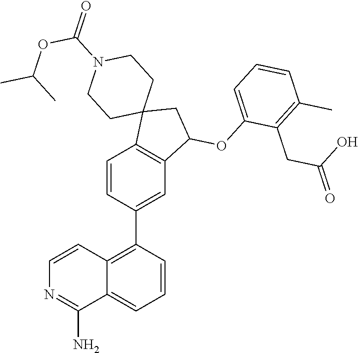 [SMILES: Cc1cccc(OC2CC3(CCN(C(=O)OC(C)C)CC3)c3ccc(-c4cccc5c(N)nccc45)cc32)c1CC(=O)O]

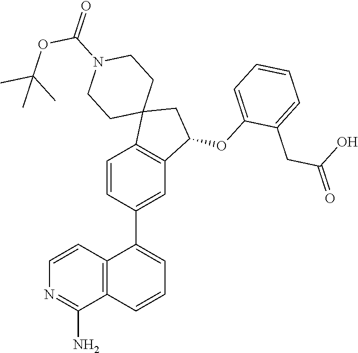 [SMILES: CC(C)(C)OC(=O)N1CCC2(CC1)C[C@H](Oc1ccccc1CC(=O)O)c1cc(-c3cccc4c(N)nccc34)ccc12]

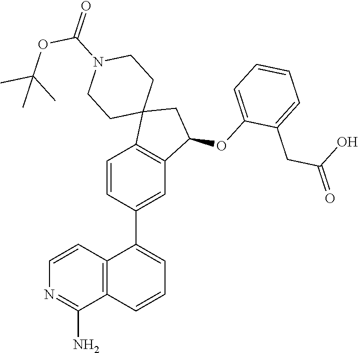 [SMILES: CC(C)(C)OC(=O)N1CCC2(CC1)C[C@@H](Oc1ccccc1CC(=O)O)c1cc(-c3cccc4c(N)nccc34)ccc12]

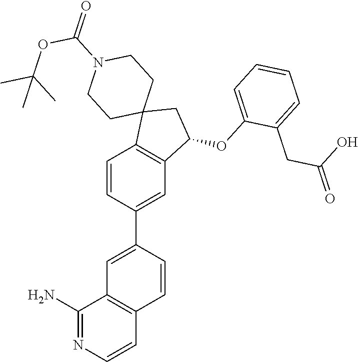 [SMILES: CC(C)(C)OC(=O)N1CCC2(CC1)C[C@H](Oc1ccccc1CC(=O)O)c1cc(-c3ccc4ccnc(N)c4c3)ccc12]

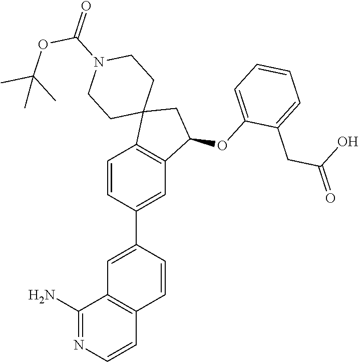 [SMILES: CC(C)(C)OC(=O)N1CCC2(CC1)C[C@@H](Oc1ccccc1CC(=O)O)c1cc(-c3ccc4ccnc(N)c4c3)ccc12]

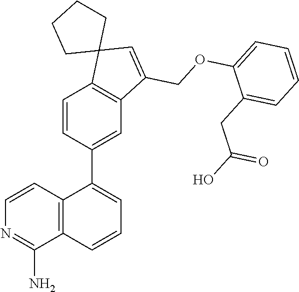 [SMILES: Nc1nccc2c(-c3ccc4c(c3)C(COc3ccccc3CC(=O)O)=CC43CCCC3)cccc12]

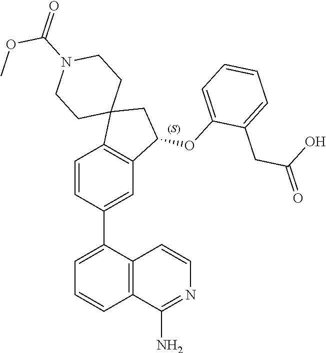

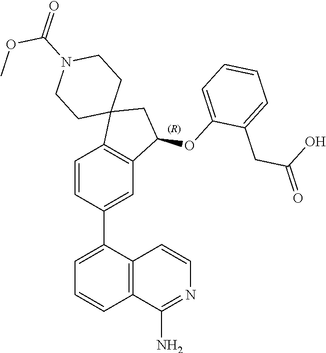

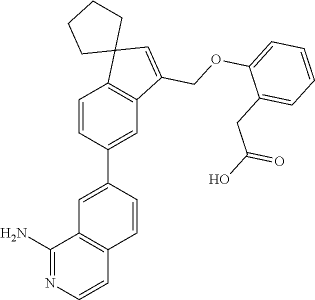 [SMILES: Nc1nccc2ccc(-c3ccc4c(c3)C(COc3ccccc3CC(=O)O)=CC43CCCC3)cc12]

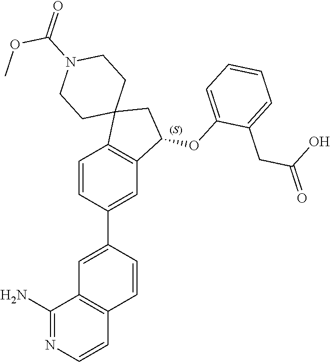

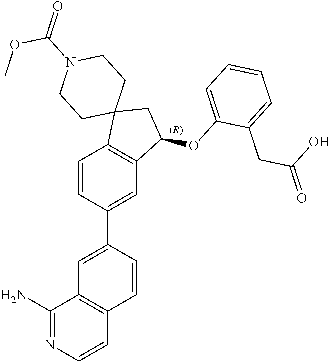

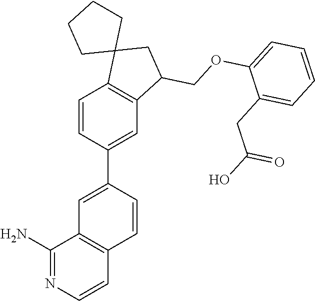 [SMILES: Nc1nccc2ccc(-c3ccc4c(c3)C(COc3ccccc3CC(=O)O)CC43CCCC3)cc12]

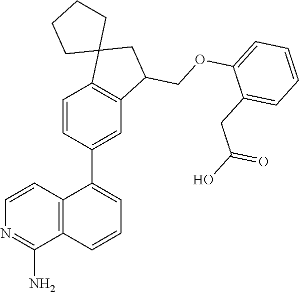 [SMILES: Nc1nccc2c(-c3ccc4c(c3)C(COc3ccccc3CC(=O)O)CC43CCCC3)cccc12]

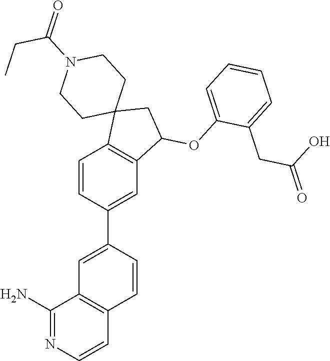 [SMILES: CCC(=O)N1CCC2(CC1)CC(Oc1ccccc1CC(=O)O)c1cc(-c3ccc4ccnc(N)c4c3)ccc12]

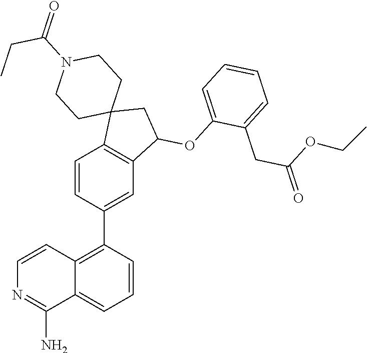 [SMILES: CCOC(=O)Cc1ccccc1OC1CC2(CCN(C(=O)CC)CC2)c2ccc(-c3cccc4c(N)nccc34)cc21]

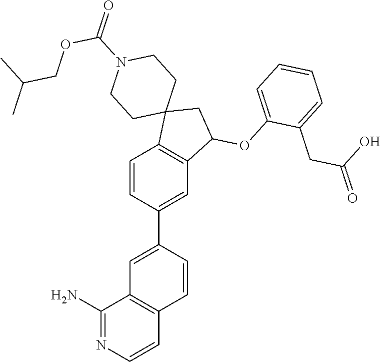 [SMILES: CC(C)COC(=O)N1CCC2(CC1)CC(Oc1ccccc1CC(=O)O)c1cc(-c3ccc4ccnc(N)c4c3)ccc12]

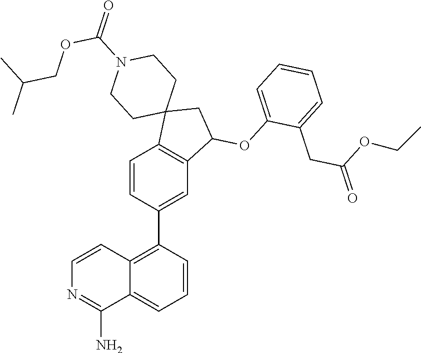 [SMILES: CCOC(=O)Cc1ccccc1OC1CC2(CCN(C(=O)OCC(C)C)CC2)c2ccc(-c3cccc4c(N)nccc34)cc21]

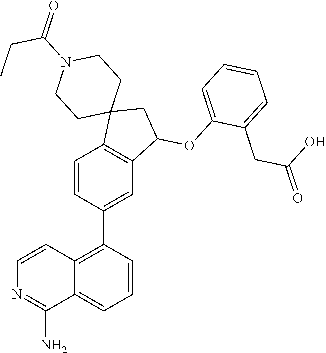 [SMILES: CCC(=O)N1CCC2(CC1)CC(Oc1ccccc1CC(=O)O)c1cc(-c3cccc4c(N)nccc34)ccc12]

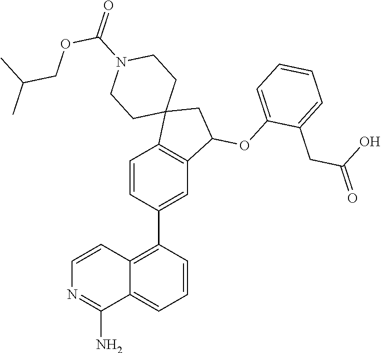 [SMILES: CC(C)COC(=O)N1CCC2(CC1)CC(Oc1ccccc1CC(=O)O)c1cc(-c3cccc4c(N)nccc34)ccc12]

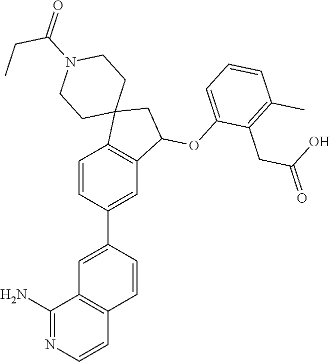 [SMILES: CCC(=O)N1CCC2(CC1)CC(Oc1cccc(C)c1CC(=O)O)c1cc(-c3ccc4ccnc(N)c4c3)ccc12]

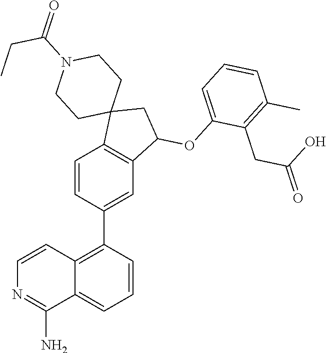 [SMILES: CCC(=O)N1CCC2(CC1)CC(Oc1cccc(C)c1CC(=O)O)c1cc(-c3cccc4c(N)nccc34)ccc12]

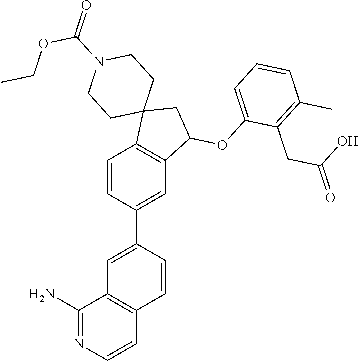 [SMILES: CCOC(=O)N1CCC2(CC1)CC(Oc1cccc(C)c1CC(=O)O)c1cc(-c3ccc4ccnc(N)c4c3)ccc12]

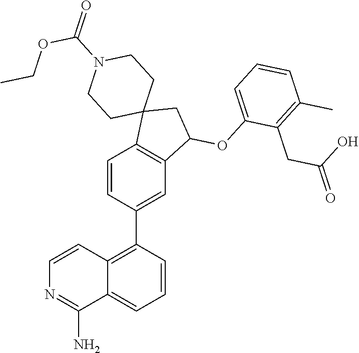 [SMILES: CCOC(=O)N1CCC2(CC1)CC(Oc1cccc(C)c1CC(=O)O)c1cc(-c3cccc4c(N)nccc34)ccc12]

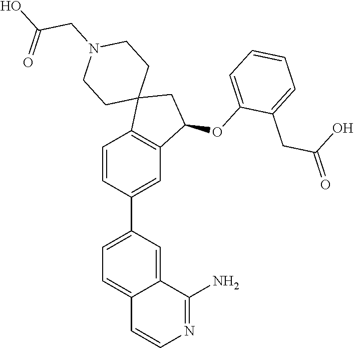 [SMILES: Nc1nccc2ccc(-c3ccc4c(c3)[C@H](Oc3ccccc3CC(=O)O)CC43CCN(CC(=O)O)CC3)cc12]

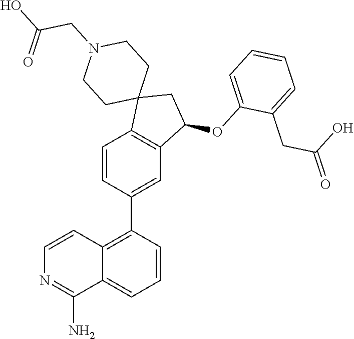 [SMILES: Nc1nccc2c(-c3ccc4c(c3)[C@H](Oc3ccccc3CC(=O)O)CC43CCN(CC(=O)O)CC3)cccc12]

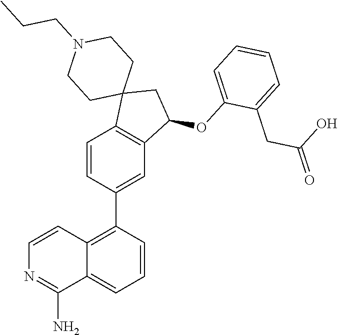 [SMILES: CCCN1CCC2(CC1)C[C@@H](Oc1ccccc1CC(=O)O)c1cc(-c3cccc4c(N)nccc34)ccc12]

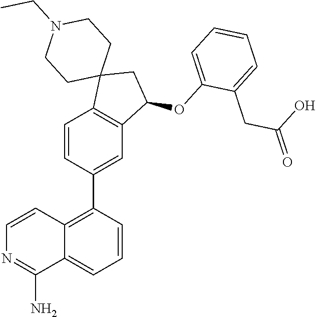 [SMILES: CCN1CCC2(CC1)C[C@@H](Oc1ccccc1CC(=O)O)c1cc(-c3cccc4c(N)nccc34)ccc12]

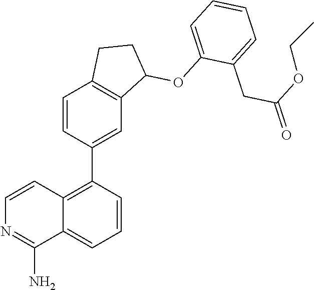 [SMILES: CCOC(=O)Cc1ccccc1OC1CCc2ccc(-c3cccc4c(N)nccc34)cc21]

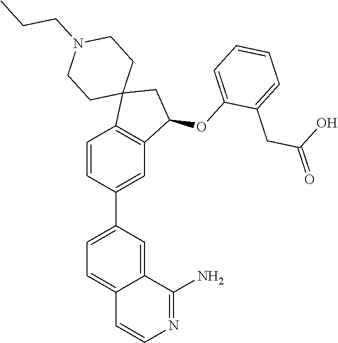 [SMILES: CCCN1CCC2(CC1)C[C@@H](Oc1ccccc1CC(=O)O)c1cc(-c3ccc4ccnc(N)c4c3)ccc12]

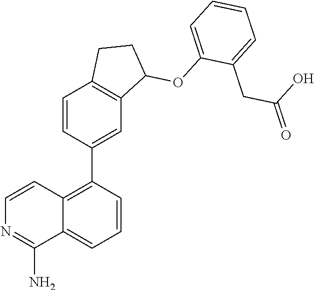 [SMILES: Nc1nccc2c(-c3ccc4c(c3)C(Oc3ccccc3CC(=O)O)CC4)cccc12]

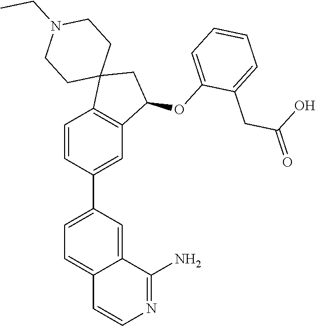 [SMILES: CCN1CCC2(CC1)C[C@@H](Oc1ccccc1CC(=O)O)c1cc(-c3ccc4ccnc(N)c4c3)ccc12]

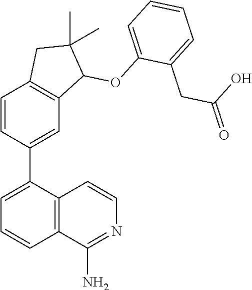 [SMILES: CC1(C)Cc2ccc(-c3cccc4c(N)nccc34)cc2C1Oc1ccccc1CC(=O)O]

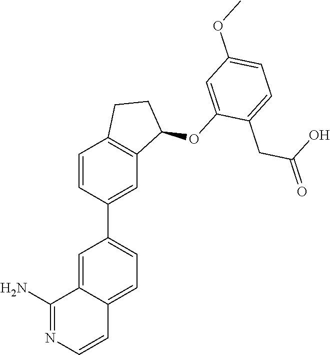 [SMILES: COc1ccc(CC(=O)O)c(O[C@@H]2CCc3ccc(-c4ccc5ccnc(N)c5c4)cc32)c1]

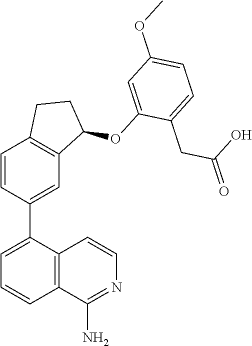 [SMILES: COc1ccc(CC(=O)O)c(O[C@@H]2CCc3ccc(-c4cccc5c(N)nccc45)cc32)c1]

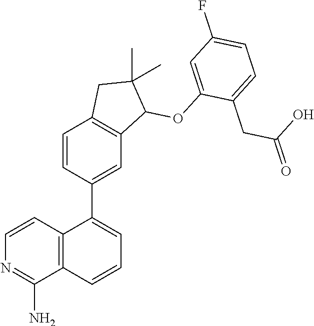 [SMILES: CC1(C)Cc2ccc(-c3cccc4c(N)nccc34)cc2C1Oc1cc(F)ccc1CC(=O)O]

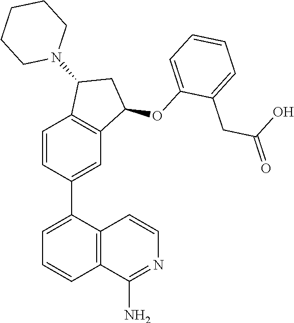 [SMILES: Nc1nccc2c(-c3ccc4c(c3)[C@H](Oc3ccccc3CC(=O)O)C[C@H]4N3CCCCC3)cccc12]

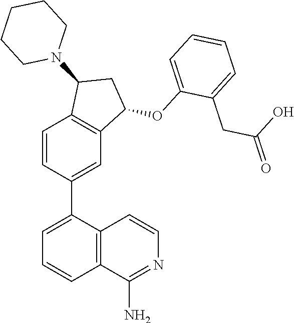 [SMILES: Nc1nccc2c(-c3ccc4c(c3)[C@@H](Oc3ccccc3CC(=O)O)C[C@@H]4N3CCCCC3)cccc12]

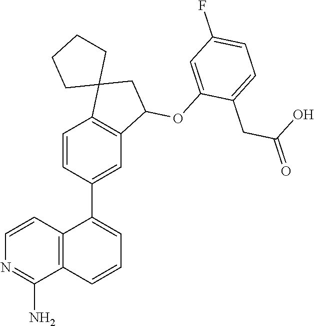 [SMILES: Nc1nccc2c(-c3ccc4c(c3)C(Oc3cc(F)ccc3CC(=O)O)CC43CCCC3)cccc12]

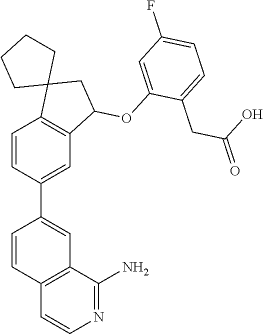 [SMILES: Nc1nccc2ccc(-c3ccc4c(c3)C(Oc3cc(F)ccc3CC(=O)O)CC43CCCC3)cc12]

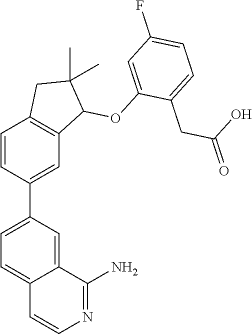 [SMILES: CC1(C)Cc2ccc(-c3ccc4ccnc(N)c4c3)cc2C1Oc1cc(F)ccc1CC(=O)O]

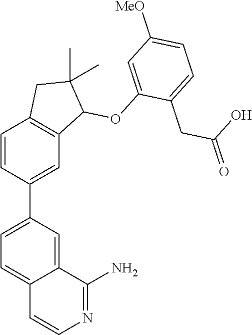 [SMILES: COc1ccc(CC(=O)O)c(OC2c3cc(-c4ccc5ccnc(N)c5c4)ccc3CC2(C)C)c1]

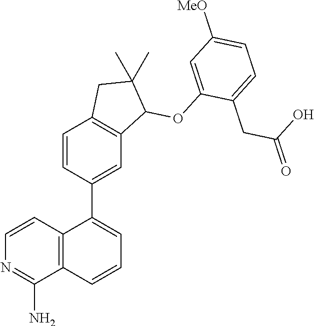 [SMILES: COc1ccc(CC(=O)O)c(OC2c3cc(-c4cccc5c(N)nccc45)ccc3CC2(C)C)c1]

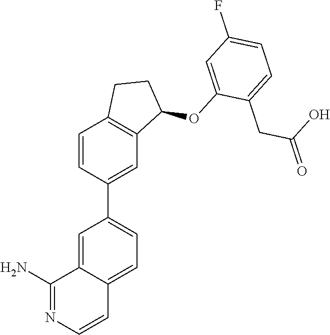 [SMILES: Nc1nccc2ccc(-c3ccc4c(c3)[C@H](Oc3cc(F)ccc3CC(=O)O)CC4)cc12]

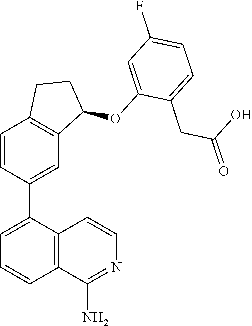 [SMILES: Nc1nccc2c(-c3ccc4c(c3)[C@H](Oc3cc(F)ccc3CC(=O)O)CC4)cccc12]

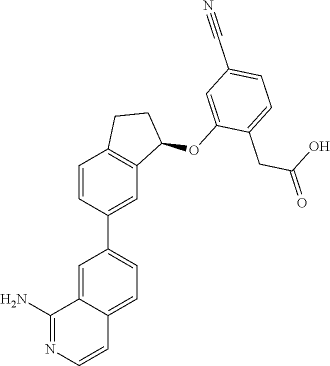 [SMILES: N#Cc1ccc(CC(=O)O)c(O[C@@H]2CCc3ccc(-c4ccc5ccnc(N)c5c4)cc32)c1]

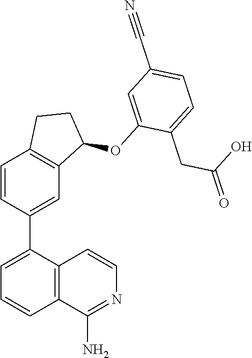 [SMILES: N#Cc1ccc(CC(=O)O)c(O[C@@H]2CCc3ccc(-c4cccc5c(N)nccc45)cc32)c1]

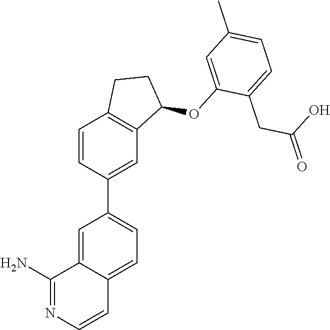 [SMILES: Cc1ccc(CC(=O)O)c(O[C@@H]2CCc3ccc(-c4ccc5ccnc(N)c5c4)cc32)c1]

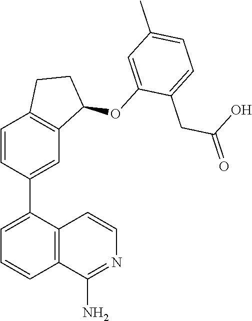 [SMILES: Cc1ccc(CC(=O)O)c(O[C@@H]2CCc3ccc(-c4cccc5c(N)nccc45)cc32)c1]

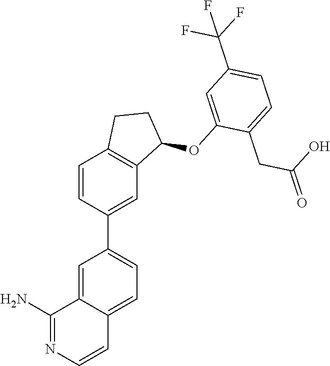 [SMILES: Nc1nccc2ccc(-c3ccc4c(c3)[C@H](Oc3cc(C(F)(F)F)ccc3CC(=O)O)CC4)cc12]

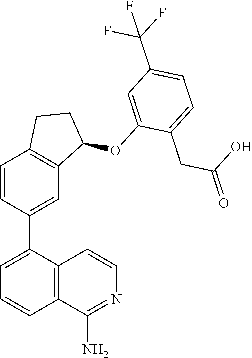 [SMILES: Nc1nccc2c(-c3ccc4c(c3)[C@H](Oc3cc(C(F)(F)F)ccc3CC(=O)O)CC4)cccc12]

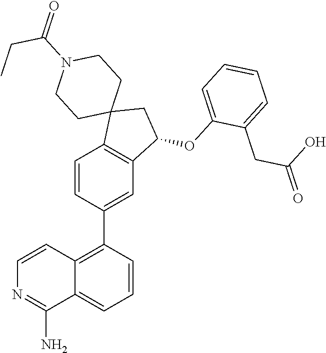 [SMILES: CCC(=O)N1CCC2(CC1)C[C@H](Oc1ccccc1CC(=O)O)c1cc(-c3cccc4c(N)nccc34)ccc12]

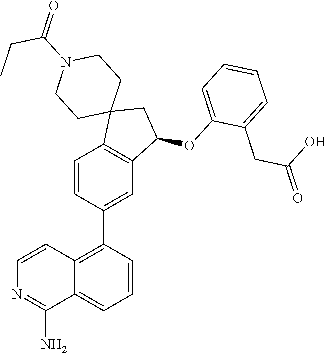 [SMILES: CCC(=O)N1CCC2(CC1)C[C@@H](Oc1ccccc1CC(=O)O)c1cc(-c3cccc4c(N)nccc34)ccc12]

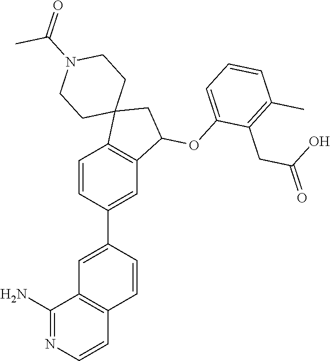 [SMILES: CC(=O)N1CCC2(CC1)CC(Oc1cccc(C)c1CC(=O)O)c1cc(-c3ccc4ccnc(N)c4c3)ccc12]

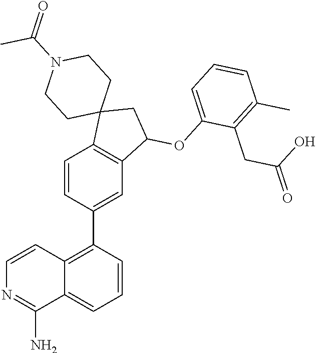 [SMILES: CC(=O)N1CCC2(CC1)CC(Oc1cccc(C)c1CC(=O)O)c1cc(-c3cccc4c(N)nccc34)ccc12]

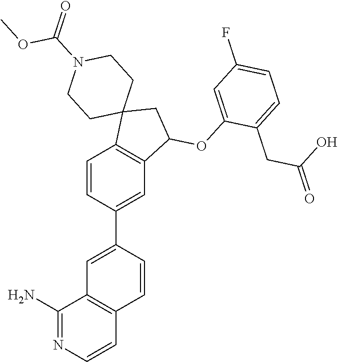 [SMILES: COC(=O)N1CCC2(CC1)CC(Oc1cc(F)ccc1CC(=O)O)c1cc(-c3ccc4ccnc(N)c4c3)ccc12]

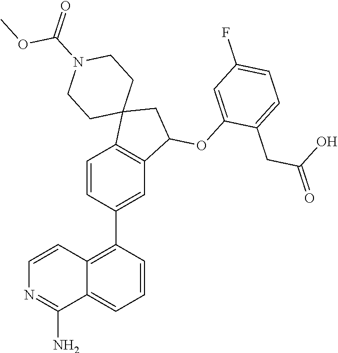 [SMILES: COC(=O)N1CCC2(CC1)CC(Oc1cc(F)ccc1CC(=O)O)c1cc(-c3cccc4c(N)nccc34)ccc12]

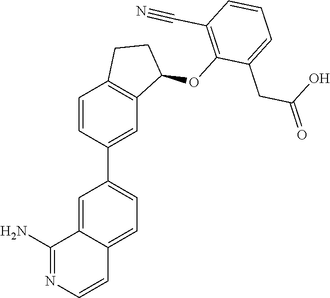 [SMILES: N#Cc1cccc(CC(=O)O)c1O[C@@H]1CCc2ccc(-c3ccc4ccnc(N)c4c3)cc21]

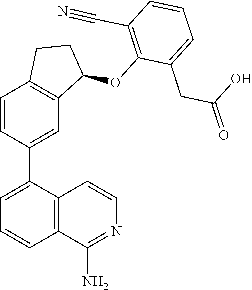 [SMILES: N#Cc1cccc(CC(=O)O)c1O[C@@H]1CCc2ccc(-c3cccc4c(N)nccc34)cc21]

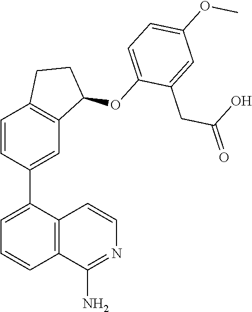 [SMILES: COc1ccc(O[C@@H]2CCc3ccc(-c4cccc5c(N)nccc45)cc32)c(CC(=O)O)c1]

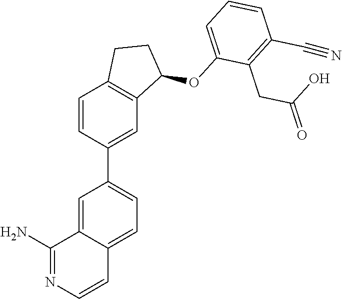 [SMILES: N#Cc1cccc(O[C@@H]2CCc3ccc(-c4ccc5ccnc(N)c5c4)cc32)c1CC(=O)O]

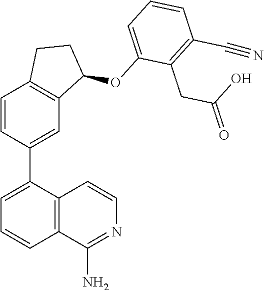 [SMILES: N#Cc1cccc(O[C@@H]2CCc3ccc(-c4cccc5c(N)nccc45)cc32)c1CC(=O)O]

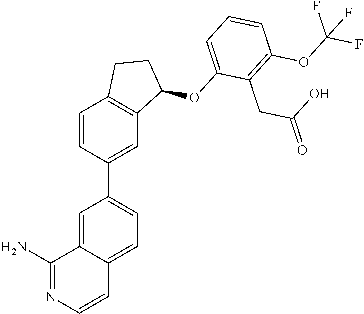 [SMILES: Nc1nccc2ccc(-c3ccc4c(c3)[C@H](Oc3cccc(OC(F)(F)F)c3CC(=O)O)CC4)cc12]

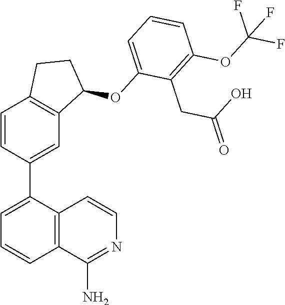 [SMILES: Nc1nccc2c(-c3ccc4c(c3)[C@H](Oc3cccc(OC(F)(F)F)c3CC(=O)O)CC4)cccc12]

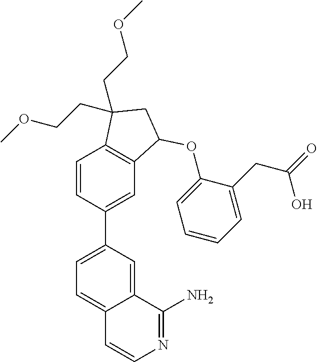 [SMILES: COCCC1(CCOC)CC(Oc2ccccc2CC(=O)O)c2cc(-c3ccc4ccnc(N)c4c3)ccc21]

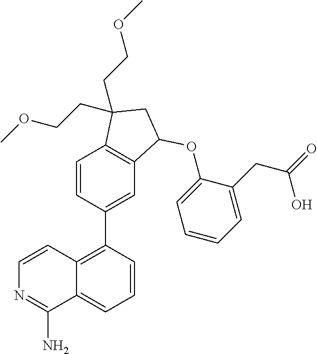 [SMILES: COCCC1(CCOC)CC(Oc2ccccc2CC(=O)O)c2cc(-c3cccc4c(N)nccc34)ccc21]

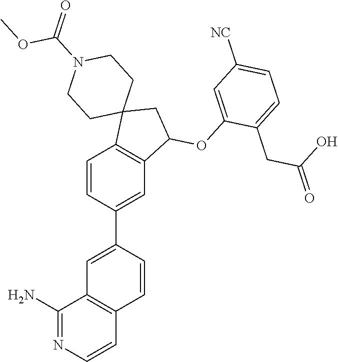 [SMILES: COC(=O)N1CCC2(CC1)CC(Oc1cc(C#N)ccc1CC(=O)O)c1cc(-c3ccc4ccnc(N)c4c3)ccc12]

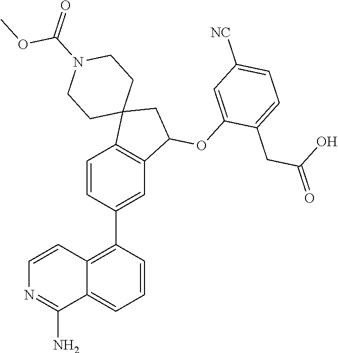 [SMILES: COC(=O)N1CCC2(CC1)CC(Oc1cc(C#N)ccc1CC(=O)O)c1cc(-c3cccc4c(N)nccc34)ccc12]

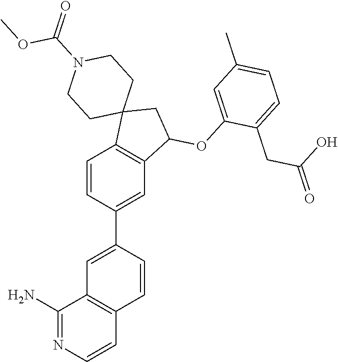 [SMILES: COC(=O)N1CCC2(CC1)CC(Oc1cc(C)ccc1CC(=O)O)c1cc(-c3ccc4ccnc(N)c4c3)ccc12]

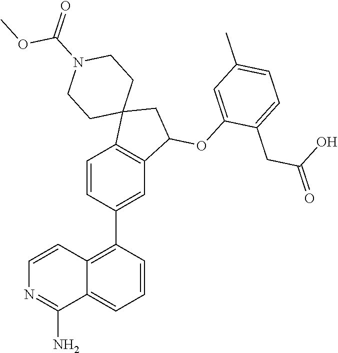 [SMILES: COC(=O)N1CCC2(CC1)CC(Oc1cc(C)ccc1CC(=O)O)c1cc(-c3cccc4c(N)nccc34)ccc12]

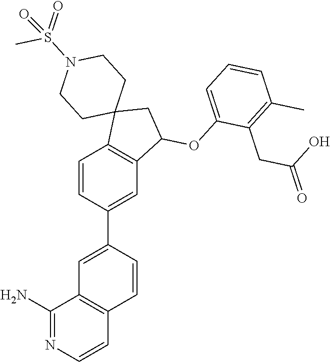 [SMILES: Cc1cccc(OC2CC3(CCN(S(C)(=O)=O)CC3)c3ccc(-c4ccc5ccnc(N)c5c4)cc32)c1CC(=O)O]

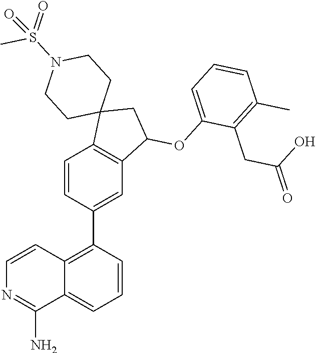 [SMILES: Cc1cccc(OC2CC3(CCN(S(C)(=O)=O)CC3)c3ccc(-c4cccc5c(N)nccc45)cc32)c1CC(=O)O]

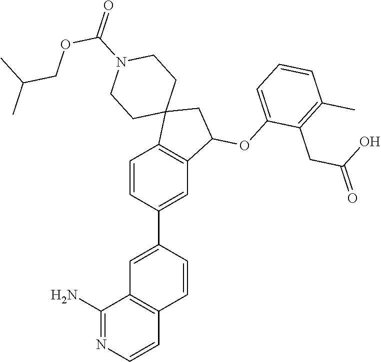 [SMILES: Cc1cccc(OC2CC3(CCN(C(=O)OCC(C)C)CC3)c3ccc(-c4ccc5ccnc(N)c5c4)cc32)c1CC(=O)O]

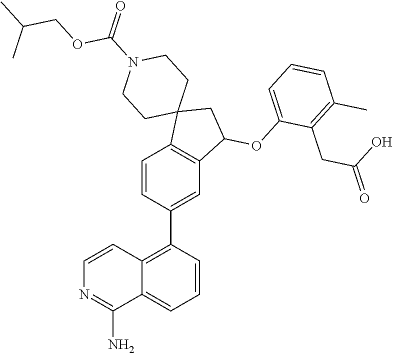 [SMILES: Cc1cccc(OC2CC3(CCN(C(=O)OCC(C)C)CC3)c3ccc(-c4cccc5c(N)nccc45)cc32)c1CC(=O)O]

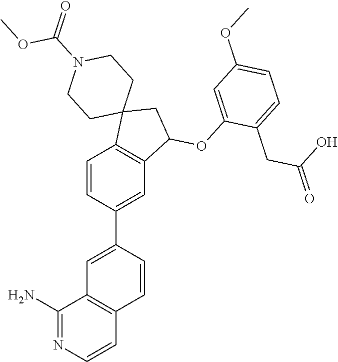 [SMILES: COC(=O)N1CCC2(CC1)CC(Oc1cc(OC)ccc1CC(=O)O)c1cc(-c3ccc4ccnc(N)c4c3)ccc12]

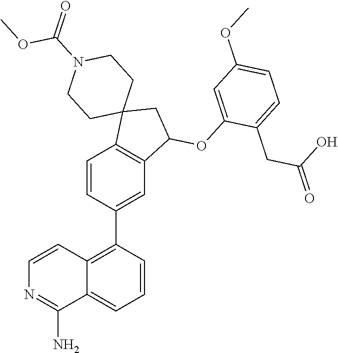 [SMILES: COC(=O)N1CCC2(CC1)CC(Oc1cc(OC)ccc1CC(=O)O)c1cc(-c3cccc4c(N)nccc34)ccc12]

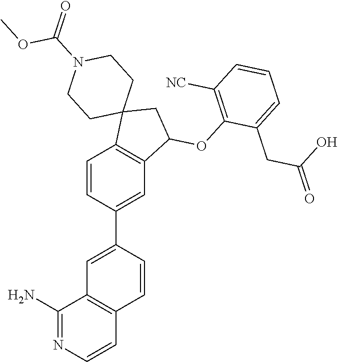 [SMILES: COC(=O)N1CCC2(CC1)CC(Oc1c(C#N)cccc1CC(=O)O)c1cc(-c3ccc4ccnc(N)c4c3)ccc12]

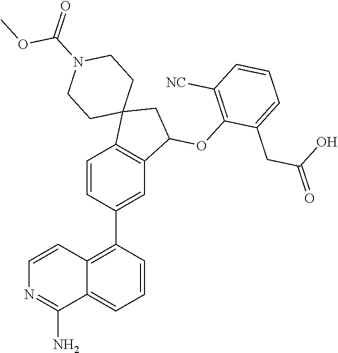 [SMILES: COC(=O)N1CCC2(CC1)CC(Oc1c(C#N)cccc1CC(=O)O)c1cc(-c3cccc4c(N)nccc34)ccc12]

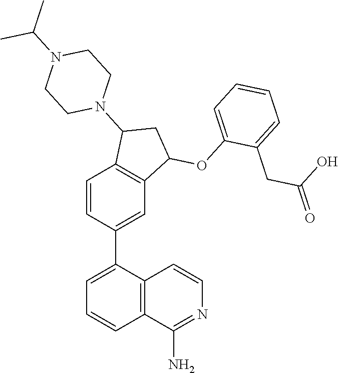 [SMILES: CC(C)N1CCN(C2CC(Oc3ccccc3CC(=O)O)c3cc(-c4cccc5c(N)nccc45)ccc32)CC1]

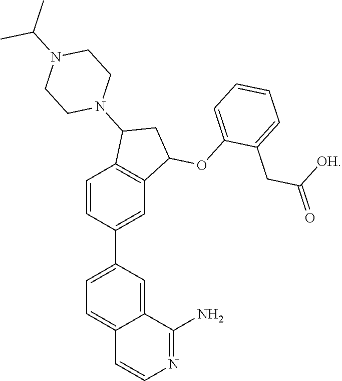 [SMILES: CC(C)N1CCN(C2CC(Oc3ccccc3CC(=O)O)c3cc(-c4ccc5ccnc(N)c5c4)ccc32)CC1]

The present invention provides also compounds having the structure of Formula (IV), and pharmaceutically acceptable salts thereof.

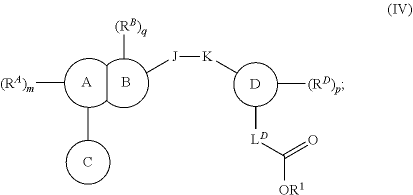

wherein:
ring

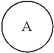

is arylene or heteroarylene;
ring

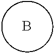

is arylene or heteroarylene;
ring

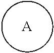

is fused to ring

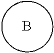

at two and only two adjacent positions;
ring

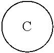

is selected from the group consisting of

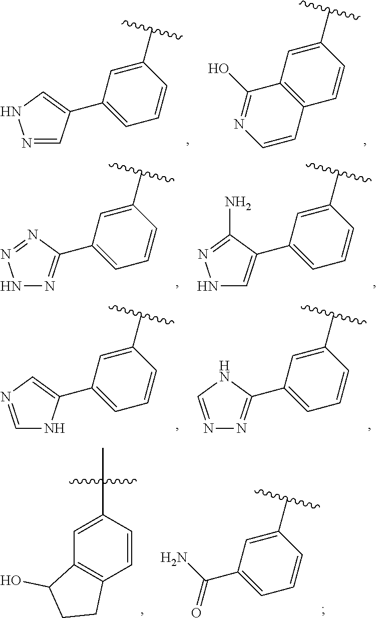

ring

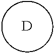

is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or

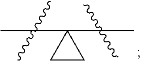 [SMILES: *C1(*)CC1]

K represents a bond, —O—, —NH—, —C(O)—, —CH₂—, —S—, —S(O)—, —SO₂—, —N(alkyl)-, —CH(alkyl)-, or —CH(cycloalkyl)-;
   wherein at least one of J and K is a bond, —C(O)—, —CH₂—, —CH₂CH₂, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl). —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl). —C(O)O(alkyl). —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl). —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{D}, independently for each occurrence, represents H, halo, hydroxyl, cyano, —NH₂. —NH(Ac), —NH(alkyl), —N(alkyl)₂, —NH(CO)(alkyl), —CH₂NH₂, —CH₂NHC(O)(alkyl). —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, p, and q are each independently 0, 1, or 2.

In certain embodiments, in the compound having the structure of Formula (IV):
ring

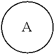

is arylene;
ring

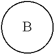

is heteroarylene;
ring

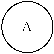

is fused to ring

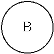

at two and only two adjacent positions;
ring

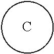

is selected from the group consisting of

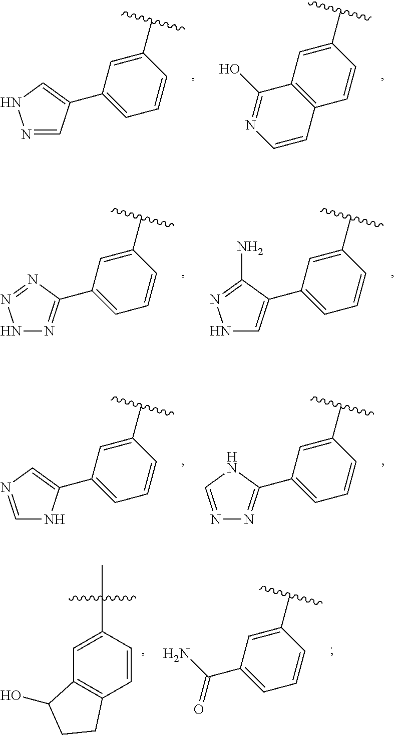

ring

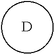

is aryl;
J represents —CH₂;
K represents a bond or —O—;
L^{D} represents —CH₂—;
R^{A} represents H;
R^{B}, independently for each occurrence, represents H, oxo, or alkyl;
R^{D} represents H;
R¹ represents H; and
m, p, and q are each independently 0, 1, or 2.

In some embodiments, the compound having the structure of Formula (IV) has the structure of formula (IVa):

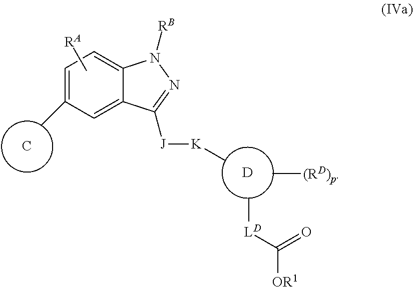

In certain embodiments, J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

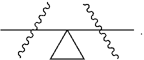 [SMILES: *C1(*)CC1]

In certain embodiments, -J-K- represents —CH₂—O— or —CH₂—.

In certain embodiments,

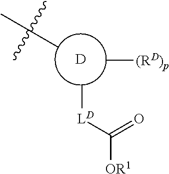

represents

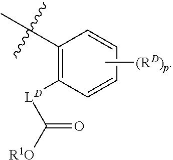

In certain embodiments, ring

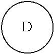

is bicyclic heteroaryl. For example, in some embodiments,

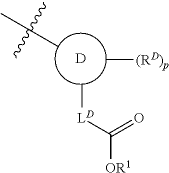

represents

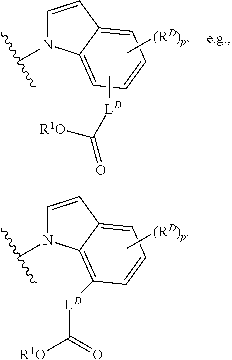

In certain embodiments, m, p, and q are each independently 0 or 1.

In certain embodiments, R¹ is H.

In certain embodiments, L^{D} represents —CH₂—.

In certain embodiments, R^{D} represents H.

In certain embodiments, R^{B}, independently for each occurrence, represents H, oxo, or alkyl. In certain embodiments, R^{B} represents cycloalkyl or heterocycloalkyl. In further embodiments, two geminal occurrences of R^{B} taken together with the atom to which they are attached form an optionally substituted spirocycloalkyl or a spiroheterocycloalkyl.

In certain embodiments, R^{A} represents H.

In certain embodiments, the compound of formula (IV) is selected from the following table of compounds, and pharmaceutically acceptable salts thereof:

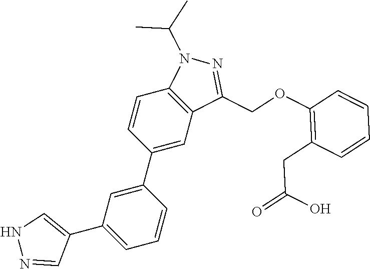 [SMILES: CC(C)n1nc(COc2ccccc2CC(=O)O)c2cc(-c3cccc(-c4cn[nH]c4)c3)ccc21]

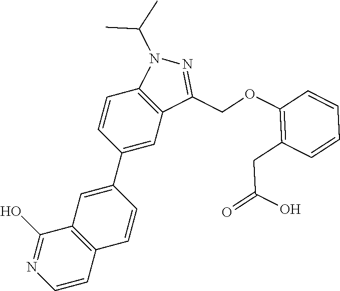 [SMILES: CC(C)n1nc(COc2ccccc2CC(=O)O)c2cc(-c3ccc4ccnc(O)c4c3)ccc21]

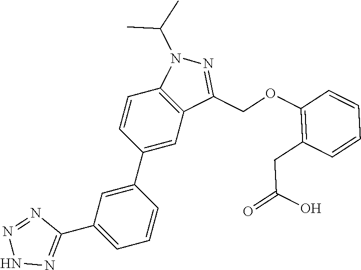 [SMILES: CC(C)n1nc(COc2ccccc2CC(=O)O)c2cc(-c3cccc(-c4nn[nH]n4)c3)ccc21]

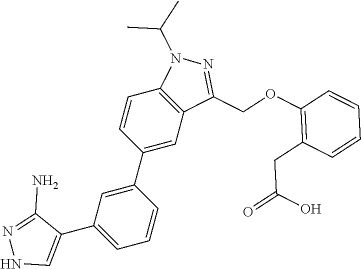 [SMILES: CC(C)n1nc(COc2ccccc2CC(=O)O)c2cc(-c3cccc(-c4c[nH]nc4N)c3)ccc21]

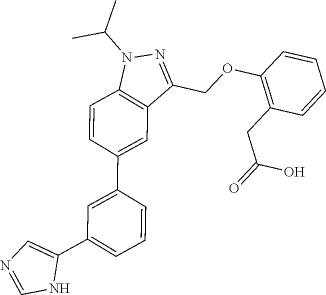 [SMILES: CC(C)n1nc(COc2ccccc2CC(=O)O)c2cc(-c3cccc(-c4cnc[nH]4)c3)ccc21]

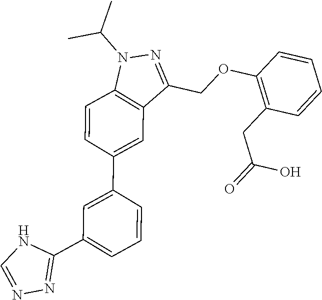 [SMILES: CC(C)n1nc(COc2ccccc2CC(=O)O)c2cc(-c3cccc(-c4nnc[nH]4)c3)ccc21]

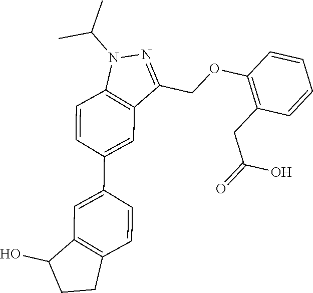 [SMILES: CC(C)n1nc(COc2ccccc2CC(=O)O)c2cc(-c3ccc4c(c3)C(O)CC4)ccc21]

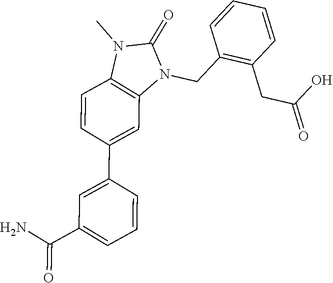 [SMILES: Cn1c(=O)n(Cc2ccccc2CC(=O)O)c2cc(-c3cccc(C(N)=O)c3)ccc21]

### Pharmaceutical Compositions

The invention provides pharmaceutical compositions, each comprising one or more compounds of the invention, or pharmaceutically acceptable salts thereof, and a pharmaceutically acceptable carrier. In certain embodiments, the pharmaceutical composition comprises a compound of the invention, or a pharmaceutically acceptable salt thereof, and a pharmaceutically acceptable carrier. In certain embodiments, the pharmaceutical composition comprises a plurality of compounds of the invention, which may include pharmaceutically acceptable salts thereof, and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of a disease or condition characterized by aberrant kallikrein activity.

Pharmaceutical compositions of the invention can be prepared by combining one or more compounds of the invention with a pharmaceutically acceptable carrier and, optionally, one or more additional pharmaceutically active agents.

### Methods of Use

The present invention provides compounds, and pharmaceutically acceptable salts thereof, that are useful for treating or preventing a disease or condition characterized by aberrant kallikrein activity.

In certain aspects, the invention provides a compound of the invention, or a pharmaceutically acceptable salt thereof, for use as a medicament.

In certain aspects, the invention provides methods of treating or preventing a disease or condition characterized by aberrant kallikrein activity. The method includes the step of administering to a subject in need thereof a therapeutically effective amount of a compound of the invention, or a pharmaceutically acceptable salt thereof, thereby treating or preventing the disease or condition characterized by aberrant kallikrein activity. By reducing kallikrein activity in the subject, the disease or condition characterized by aberrant kallikrein activity is treated.

Alternatively, in certain aspects, the invention provides a compound of the invention, or a pharmaceutically acceptable salt thereof, for treatment of a disease or condition characterized by aberrant kallikrein activity.

Alternatively, in certain aspects, the invention provides the use of a compound of the invention, or a pharmaceutically acceptable salt thereof, for the manufacture of a medicament for use in treatment of a disease or condition characterized by aberrant kallikrein activity.

As used herein, a “disease or condition characterized by aberrant kallikrein activity” refers to any disease or condition in which it is desirable to reduce kallikrein activity. For example, it may be desirable to reduce kallikrein activity in the setting of inappropriate activation or hyperactivation of kallikrein.

In certain embodiments, the disease or condition characterized by aberrant kallikrein activity is characterized by aberrant kallikrein-related peptidase 5 (KLK5) activity.

In certain embodiments, the disease or condition characterized by aberrant kallikrein activity is a skin disease.

In certain embodiments, the skin disease is eczema, atopic eczema, atopic dermatitis, ichthyosiform (scaly) erythroderma (IE), rosacea, or a skin infection.

In certain embodiments, the disease or condition is Netherton Syndrome.

In certain embodiments, the disease or condition characterized by aberrant kallikrein activity is selected from the group consisting of hypersensitivity of the immune system (atopy), hyper IgE syndrome, allergies (including allergies to food and airborne agents), asthma, allergic asthma, chronic inflammation, rhinitis, conjunctivitis, angioedema, eosinophilia, eosinophilic esophagitis, growth delay, failure to thrive, trichorrhexis invaginata (TI), respiratory tract infections, systemic infections and gastrointestinal disorders.

In certain embodiments, the disease or condition is cancer. The cancer may be selected from ovarian cancer, breast cancer, prostate cancer, bladder cancer, cervical cancer, glioblastoma multiforme, and neuroblastoma.

In certain aspects, the invention provides methods of treating or preventing a disease or condition characterized by aberrant kallikrein activity comprising administering to a subject in need thereof a therapeutically effective amount of a compound, or a pharmaceutically acceptable salt thereof; wherein the compound is selected from the group consisting of:

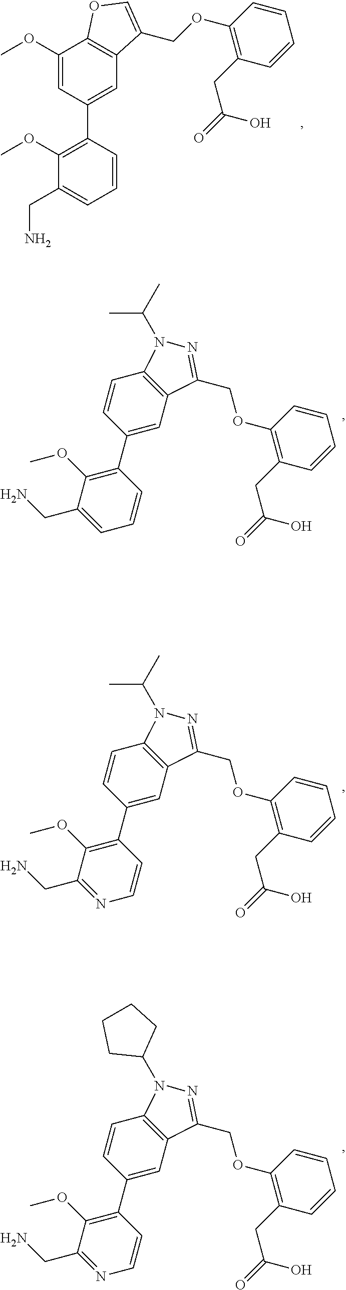 [SMILES: COc1c(CN)cccc1-c1cc(OC)c2occ(COc3ccccc3CC(=O)O)c2c1 ; COc1c(CN)cccc1-c1ccc2c(c1)c(COc1ccccc1CC(=O)O)nn2C(C)C ; COc1c(-c2ccc3c(c2)c(COc2ccccc2CC(=O)O)nn3C(C)C)ccnc1CN ; COc1c(-c2ccc3c(c2)c(COc2ccccc2CC(=O)O)nn3C2CCCC2)ccnc1CN]

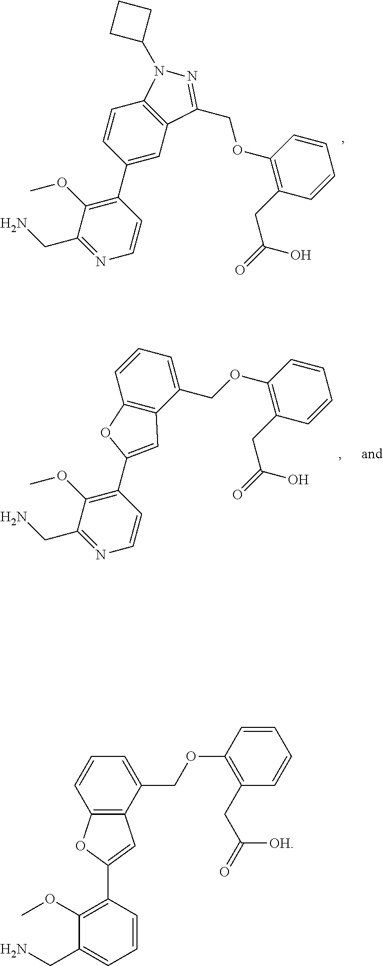 [SMILES: COc1c(-c2ccc3c(c2)c(COc2ccccc2CC(=O)O)nn3C2CCC2)ccnc1CN ; COc1c(-c2cc3c(COc4ccccc4CC(=O)O)cccc3o2)ccnc1CN ; COc1c(CN)cccc1-c1cc2c(COc3ccccc3CC(=O)O)cccc2o1]

In certain such embodiments, the disease or condition characterized by aberrant kallikrein activity is characterized by aberrant kallikrein-related peptidase 5 (KLK5) activity.

In certain embodiments, the disease or condition characterized by aberrant kallikrein activity is a skin disease.

In certain embodiments, the skin disease is eczema, atopic eczema, atopic dermatitis, ichthyosiform (scaly) erythroderma (IE), rosacea, or a skin infection.

In certain embodiments, the disease or condition is Netherton Syndrome.

In certain embodiments, the disease or condition characterized by aberrant kallikrein activity is selected from the group consisting of rhinitis, conjunctivitis, angioedema, cosinophilia, eosinophilic esophagitis, growth delay, failure to thrive, trichorrhexis invaginata (TI), respiratory tract infections, systemic infections and gastrointestinal disorders.

In certain embodiments, the disease or condition characterized by aberrant kallikrein activity is selected from the group consisting of hypersensitivity of the immune system (atopy), hyper IgE syndrome, allergies (including allergies to food and airborne agents), asthma, allergic asthma, and chronic inflammation.

In certain embodiments, the disease or condition is cancer. The cancer may be selected from ovarian cancer, breast cancer, prostate cancer, bladder cancer, cervical cancer, glioblastoma multiforme, and neuroblastoma.

### Formulations, Routes of Administration, and Dosing

The compounds of the invention, and pharmaceutically acceptable salts thereof, can be formulated as pharmaceutical compositions and administered to a mammalian host, such as a human patient, in a variety of forms adapted to the chosen route of administration, e.g., orally or parenterally, by intravenous, intraperitoneal, intramuscular, topical, or subcutaneous routes. Additional routes of administration are also contemplated by the invention.

Thus, the compounds of the invention may be systemically administered, e.g., orally, in combination with a pharmaceutically acceptable carrier such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more pharmaceutically acceptable carriers and used in the form of ingestible tablets, buccal tablets, troches, pills, capsules, pastes, creams, ointments, elixirs, suspensions, syrups, wafers, and the like. Such compositions and preparations should contain at least 0.1% of active compound. The percentage of the active compound in the compositions and preparations may, of course, be varied and may conveniently be between about 2% to about 60% of the weight of a given unit dosage form. The amount of active compound in such therapeutically useful compositions is such that an effective dosage level will be obtained, such as after administration to a human subject.

The tablets, troches, pills, capsules, and the like may also contain the following pharmaceutically acceptable carriers: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds of the invention may also be administered intravenously or intraperitoneally by infusion or injection. Solutions of can be prepared in water or physiologically acceptable aqueous solution, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising a compound of the invention which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating a compound of the invention in the required amount in the appropriate solvent with various of the other ingredients enumerated above, as required, followed by filter sterilization. In the case of sterile powders for the preparation of sterile injectable solutions, methods of preparation can include vacuum drying and the freeze drying techniques, which yield a powder of the active ingredient plus any additional desired ingredient present in the previously sterile-filtered solutions.

For topical administration, compounds of the invention may be applied in pure form, i.e., when they are liquids. However, it will generally be desirable to administer them to the skin as compositions or formulations, in combination with a pharmaceutically acceptable carrier for use in dermatological applications, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are known in the art; for example, see Jacquet et al. (U.S. Pat. No. 4,608,392; incorporated herein by reference), Geria (U.S. Pat. No. 4,992,478; incorporated herein by reference), Smith et al. (U.S. Pat. No. 4,559,157; incorporated herein by reference), and Wortzman (U.S. Pat. No. 4,820,508; incorporated herein by reference).

Useful dosages of the compounds of the invention can be determined, at least initially, by comparing their in vitro activity and in vivo activity in animal models. Methods for the extrapolation of effective dosages in mice, and other animals, to humans are known in the art; for example, see U.S. Pat. No. 4,938,949 (incorporated herein by reference).

The amount of a compound of the invention, or pharmaceutically acceptable salt thereof, required for use in treatment will vary not only with the particular compound or salt selected but also with the route of administration, the nature of the condition being treated, and the age and condition of the patient and will be ultimately at the discretion of the attendant physician or clinician.

In general, however, a suitable dose will be in the range of from about 0.5 to about 100 mg/kg body weight of the recipient per day, e.g., from about 3 to about 90 mg/kg of body weight per day, from about 6 to about 75 mg per kilogram of body weight per day, from about of 10 to about 60 mg/kg of body weight per day, or from about 15 to about 50 mg/kg of body weight per day.

Compounds of the invention, or pharmaceutically acceptable salts thereof, can be conveniently formulated in unit dosage form; for example, containing 5 to 1000 mg, 10 to 750 mg, or 50 to 500 mg of active ingredient per unit dosage form. In one embodiment, the invention provides a composition comprising a compound of the invention, or pharmaceutically acceptable salts thereof, formulated in such a unit dosage form. The desired dose may conveniently be presented in a single dose or as divided doses to be administered at appropriate intervals, for example, as two, three, four or more sub-doses per day. The sub-dose itself may be further divided. e.g., into a number of discrete loosely spaced administrations.

Compounds of the invention, or pharmaceutically acceptable salts thereof, can also be administered in combination with other therapeutic agents, for example, other agents that are useful for treating or preventing a disease or condition characterized by aberrant kallikrein activity. Compounds of the invention, or pharmaceutically acceptable salts thereof, can also be administered in combination with other therapeutic agents, for example, other agents that are useful for treating or preventing a disease or condition characterized by aberrant KLK5 activity. In certain embodiments, compounds of the invention, or pharmaceutically acceptable salts thereof, can also be administered in combination with one or more other therapeutic agents that are useful for treating or preventing Netherton Syndrome.

Other delivery systems can include time-release, delayed release, or sustained release delivery systems such as are well-known in the art. Such systems can avoid repeated administrations of the active compound, increasing convenience to the subject and the physician. Many types of release delivery systems are available and known to those of ordinary skill in the art. Use of a long-term sustained release implant may be desirable. Long-term release, as used herein, means that the delivery system or is implant constructed and arranged to deliver therapeutic levels of the active ingredient for at least 30 days, and preferably 60 days.

In certain embodiments, a compound of the invention is formulated for intraocular administration, for example direct injection or insertion with or in association with an intraocular medical device. In certain embodiments, a compound of the invention is formulated as an ophthalmic solution. In certain embodiments, a compound of the invention can be administered via ocular delivery, for example, by local ocular administration, including topical, intravitreal, periocular, transscleral, retrobulbar, juxtascleral, suprachoroidal, or sub-tenon administration. A compound of the invention can be administered via ocular delivery either alone or in combination with one or more additional therapeutic agents.

The compounds of the invention may be formulated for depositing into a medical device, which may include any of a variety of conventional grafts, stents, including stent grafts, catheters, balloons, baskets, or other device that can be deployed or permanently implanted within a body lumen. As a particular example, it would be desirable to have devices and methods which can deliver compounds of the invention to the region of a body which has been treated by interventional technique.

In exemplary embodiment, a compound of the invention may be deposited within a medical device, such as a stent, and delivered to the treatment site for treatment of a portion of the body.

Stents have been used as delivery vehicles for therapeutic agents (i.e., drugs). Intravascular stents are generally permanently implanted in coronary or peripheral vessels. Stent designs include those of U.S. Pat. No. 4,733,655 (Palmaz), U.S. Pat. No. 4,800,882 (Gianturco), or U.S. Pat. No. 4,886,062 (Wiktor). Such designs include both metal and polymeric stents, as well as self-expanding and balloon-expandable stents. Stents may also be used to deliver a drug at the site of contact with the vasculature, as disclosed in U.S. Pat. No. 5,102,417 (Palmaz). U.S. Pat. No. 5,419,760 (Narciso, Jr.), U.S. Pat. No. 5,429,634 (Narciso. Jr.), and in International Patent Application Nos. WO 91/12779 (Medtronic, Inc.) and WO 90/13332 (Cedars-Sanai Medical Center), for example.

The term “deposited” means that the compound is coated, adsorbed, placed, or otherwise incorporated into the device by methods known in the art. For example, the compound may be embedded and released from within (“matrix type”) or surrounded by and released through (“reservoir type”) polymer materials that coat or span the medical device. In the latter example, the compound may be entrapped within the polymer materials or coupled to the polymer materials using one or more the techniques for generating such materials known in the art. In other formulations, the compound may be linked to the surface of the medical device without the need for a coating, for example by means of detachable bonds, and release with time or can be removed by active mechanical or chemical processes. In other formulations, the compound may be in a permanently immobilized form that presents the compound at the implantation site.

In certain embodiments, the compounds of the invention may be incorporated with polymer compositions during the formation of biocompatible coatings for medical devices, such as stents. The coatings produced from these components are typically homogeneous and are useful for coating a number of devices designed for implantation.

The polymer may be either a biostable or a bioabsorbable polymer depending on the desired rate of release or the desired degree of polymer stability, but frequently a bioabsorbable polymer is preferred for this embodiment since, unlike a biostable polymer, it will not be present long after implantation to cause any adverse, chronic local response. Bioabsorbable polymers that could be used include, but are not limited to, poly(L-lactic acid), polycaprolactone, polyglycolide (PGA), poly(lactide-co-glycolide) (PLLA/PGA), poly(hydroxybutyrate), poly(hydroxybutyrate-co-valerate), polydioxanone, polyorthoester, polyanhydride, poly(glycolic acid), poly(D-lactic acid), poly(L-lactic acid), poly(D, L-lactic acid), poly(D, L-lactide) (PLA), poly (L-lactide) (PLLA), poly(glycolic acid-co-trimethylene carbonate) (PGA/PTMC), polyethylene oxide (PEO), polydioxanone (PDS), polyphosphoester, polyphosphoester urethane, poly(amino acids), cyanoacrylates, poly(trimethylene carbonate), poly(iminocarbonate), copoly(ether-esters) (e.g., PEO/PLA), polvalkylene oxalates, polyphosphazenes and biomolecules such as fibrin, fibrinogen, cellulose, starch, collagen and hyaluronic acid, polyepsilon caprolactone, polyhydroxy butyric acid, polyorthoesters, polyacetals, polydihydropyrans, polycyanoacrylates, cross linked or amphipathic block copolymers of hydrogels, and other suitable bioabsorbable polymers known in the art. Also, biostable polymers with a relatively low chronic tissue response such as polyurethanes, silicones, and polyesters could be used, and other polymers could also be used if they can be dissolved and cured or polymerized on the medical device such as polyolefins, polyisobutylene and ethylene-alphaolefin copolymers; acrylic polymers and copolymers, vinyl halide polymers and copolymers, such as polyvinyl chloride; polyvinylpyrrolidone; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers. ABS resins, and ethylene-vinyl acetate copolymers; pyran copolymer; polyhydroxy-propyl-methacrylamide-phenol; polyhydroxyethyl-aspartamide-phenol, polyethyleneoxide-polylysine substituted with palmitoyl residues; polyamides, such as Nylon 66 and polycaprolactam; alkyd resins, polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Polymers and semipermeable polymer matrices may be formed into shaped articles, such as valves, stents, tubing, prostheses and the like.

In certain embodiments of the invention, the compound of the invention is coupled to a polymer or semipermeable polymer matrix that is formed as a stent or stent-graft device.

Typically, polymers are applied to the surface of an implantable device by spin coating, dipping, or spraying. Additional methods known in the art can also be utilized for this purpose. Methods of spraying include traditional methods as well as microdeposition techniques with an inkjet type of dispenser. Additionally, a polymer can be deposited on an implantable device using photo-patterning to place the polymer on only specific portions of the device. This coating of the device provides a uniform layer around the device which allows for improved diffusion of various analytes through the device coating.

In certain embodiments of the invention, the compounds of the invention are formulated for release from the polymer coating into the environment in which the medical device is placed. Preferably, the compound is released in a controlled manner over an extended time frame (e.g., months) using at least one of several well-known techniques involving polymer carriers or layers to control elution. Some of these techniques are described in U.S. Patent Application 2004/0243225A1, the entire disclosure of which is incorporated herein in its entirety.

Moreover, as described for example in U.S. Pat. No. 6,770,729, which is incorporated herein in its entirety, the reagents and reaction conditions of the polymer compositions can be manipulated so that the release of the compound from the polymer coating can be controlled. For example, the diffusion coefficient of the one or more polymer coatings can be modulated to control the release of the compound from the polymer coating. In a variation on this theme, the diffusion coefficient of the one or more polymer coatings can be controlled to modulate the ability of an analyte that is present in the environment in which the medical device is placed (e.g., an analyte that facilitates the breakdown or hydrolysis of some portion of the polymer) to access one or more components within the polymer composition (and for example, thereby modulate the release of the compound from the polymer coating). Yet another embodiment of the invention includes a device having a plurality of polymer coatings, each having a plurality of diffusion coefficients. In such embodiments of the invention, the release of the compound from the polymer coating can be modulated by the plurality of polymer coatings.

In yet another embodiment of the invention, the release of the compound from the polymer coating is controlled by modulating one or more of the properties of the polymer composition, such as the presence of one or more endogenous or exogenous compounds, or alternatively, the pH of the polymer composition. For example, certain polymer compositions can be designed to release a compound in response to a decrease in the pH of the polymer composition.

### Kits

The invention also provides a kit, comprising a compound of the invention, or a pharmaceutically acceptable salt thereof, packaging material, and instructions for administering the compound of the invention, or the pharmaceutically acceptable salt thereof, to a mammal to treat or prevent a disease or condition characterized by aberrant kallikrein activity. In one embodiment, the mammal is a human. In one embodiment, the disease or condition is Netherton's Syndrome.

The invention also provides a kit, comprising a compound of the invention, or a pharmaceutically acceptable salt thereof, at least one other therapeutic agent, packaging material, and instructions for administering the compound of the invention or the pharmaceutically acceptable salt thereof and the other therapeutic agent or agents to a mammal to treat or prevent a disease or condition characterized by aberrant kallikrein activity. In one embodiment, the mammal is a human. In certain embodiments, the disease or condition is Netherton's Syndrome.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof.

### EXAMPLES

Having now described the present invention in detail, the same will be more clearly understood by reference to the following examples, which are included herewith for purposes of illustration only and are not intended to be limiting of the invention.

### List of Purification Methods Used in the Following Schemes

The purification methods used in the schemes described herein are as follows.

### Method-A

Normal phase flash column chromatography [silica gel (120 g), eluting with EtOAc in hexane from 0-50%]

### Method-B

Normal phase flash column chromatography [silica gel (24 g), eluting with EtOAc in hexane from 0-40%]

### Method-C

Normal phase flash column chromatography [silica gel (12 g), eluting with EtOAc in hexane from 0-40%]

### Method-D

Normal phase flash column chromatography [silica gel (24 g), eluting with MeOH in DCM from 0-100%]

### Method-E

Normal phase flash column chromatography [silica gel (24 g), eluting with DMA-80 (dichloromethane 80%, methanol 18%, and conc ammonium hydroxide 2%) in DCM from 0-70%]

### Method-F

Normal phase flash column chromatography [silica gel (24 g), eluting with DMA-80 in DCM from 0-100%]

### Method-G

Reverse-phase column chromatography [C-18 column (50 g), eluting with ACN in water (containing 0.1% HCl) from 0-100%]

### Method-H

Normal phase flash column chromatography [silica gel (24 g), eluting with EtOAc in hexane from 0-80%]

### Method-I

Normal phase flash column chromatography [silica gel (80 g), eluting with EtOAc in hexane from 0-100%]

### Method-J

Normal phase flash column chromatography [silica gel (24 g), eluting with EtOAc in hexane from 0-100%]

### Method-K

Normal phase flash column chromatography [silica gel (40 g), eluting with EtOAc in hexane from 0-50%]

### Method-L

Normal phase flash column chromatography [silica gel (24 g), eluting with EtOAc in hexane from 0-40%]

### Method-M

Reverse-phase column chromatography [C-18 column (30 g), eluting with ACN in water (containing 0.1% HCl) from 0-100%]

### Method-N

Normal phase flash column chromatography [silica gel (40 g), eluting with EtOAc in hexane from 0-60%]

### Method-O

Normal phase flash column chromatography [silica gel (40 g), eluting with EtOAc in hexane from 0-100%]

### Method-P

Normal phase flash column chromatography [silica gel (24 g), eluting with EtOAc in hexane from 0-50%]

### Method-Q

Normal phase flash column chromatography [silica gel (80 g), eluting with EtOAc in hexane from 0-40%]

### Method-R

Normal phase flash column chromatography [silica gel (12 g), eluting with DMA-80 in DCM from 0-100%]

### Method-S

Normal phase flash column chromatography [silica gel (24 g), eluting with EtOAc in hexane from 0-30%]

### Method-T

Normal phase flash column chromatography [silica gel (12 g), eluting with DMA-80 in DCM from 0-50%]

### Method-U

Normal phase flash column chromatography [silica gel (12 g), eluting with EtOAc in hexane from 0-100%]

### Method-V

Normal phase flash column chromatography [silica gel (12 g), eluting with EtOAc in hexane from 0-50%]

### Method-W

Normal phase flash column chromatography [silica gel (80 g), eluting with DMA-80 in DCM from 0-100%]

### Method-X

Normal phase flash column chromatography [silica gel (24 g), eluting with EtOAc in hexane from 0-60%]

### Method-Y

Normal phase flash column chromatography [silica gel (12 g), eluting with MeOH in DCM from 0-50%]

### Method-Z

Reverse-phase column chromatography [C-18 column (120 g), eluting with ACN in water from 0-60% then 60-10%]

### Method-AA

Normal phase flash column chromatography [silica gel (120 g), eluting with EtOAc in hexane from 0-40%]

### Method-AB

Normal phase flash column chromatography [silica gel (40 g), eluting with EtOAc in hexane from 0-80%]

### Method-AC

Normal phase flash column chromatography [silica gel (24 g), eluting with EtOAc in hexane from 0-70%]

### Method-AD

Normal phase flash column chromatography [silica gel (120 g), eluting with EtOAc in hexane from 0-40%] followed by reverse-phase column chromatography [C-18 column (120 g), eluting with ACN in water from 0-100%]

### Method-AE

Normal phase flash column chromatography [silica gel (12 g), eluting with DMA-80 in DCM from 0-70%]

### Method-AF

Normal phase flash column chromatography [silica gel (40 g), eluting with EtOAc in hexane from 0-40%]

### Method-AG

Normal phase flash column chromatography [silica gel (12 g), eluting with EtOAc in hexane from 0-70%]

### Method-AH

Normal phase flash column chromatography [silica gel (12 g), eluting with DCM in EtOAc/MeOH (9:1) from 0-60%]

### Method-AI

Normal phase flash column chromatography [silica gel (24 g), eluting with DMA-50 (dichloromethane 50%, methanol 40%, and conc ammonium hydroxide 10%) in DCM from 0-70%]

### Method-AJ

Normal phase flash column chromatography [silica gel (120 g), eluting with EtOAc in hexane from 0-100%]

### Method-AK

Normal phase flash column chromatography [silica gel (24 g), eluting with DMA-50 in DCM from 0-50%]

### Method-AL

Normal phase flash column chromatography [silica gel (120 g), eluting with EtOAc in hexane from 0-70%]

### Method-AM

Normal phase flash column chromatography [silica gel (24 g), eluting with DMA-80 in DCM from 0-50%]

### Method-AN

Normal phase flash column chromatography [silica gel (120 g), eluting with EtOAc in n-heptane from 0% to 10%]

### Method-AO

Normal phase flash column chromatography [silica gel (80 g), eluting with EtOAc in hexane from 0-50%]

### Method-AP

Normal phase flash column chromatography [silica gel (12 g), eluting with DMA-80 in DCM from 0-60%]

### Method-AQ

Normal phase flash column chromatography [silica gel (12 g), eluting with EtOAc in hexane from 0-60%]

### Method-AR

Normal phase flash column chromatography [silica gel (120 g), eluting with EtOAc in hexane from 0-60%]

### Method-AS

Normal phase flash column chromatography [silica gel (80 g), eluting with EtOAc in hexane from 0-60%]

### Method-AT

Normal phase flash column chromatography [silica gel (40 g), eluting with MeOH in DCM from 0-30%]

### Method-AU

Normal phase flash column chromatography [silica gel (120 g), eluting with EtOAc in hexane from 0-25%]

### Method-AV

Normal phase flash column chromatography [silica gel (40 g), eluting with EtOAc in hexane from 0-25%]

### Method-AW

Normal phase flash column chromatography [silica gel (40 g), eluting with EtOAc/MeOH (9:1) in hexane from 0-50%]

### Method-AX

Normal phase flash column chromatography [silica gel (12 g), eluting with EtOAc in hexane from 0-25%]

### Method-AY

Normal phase flash column chromatography [silica gel (120 g), eluting with EtOAc in n-heptane from 0% to 15%]

### Method-AZ

Normal phase flash column chromatography [silica gel (120 g), eluting with EtOAc in n-heptane from 0% to 5%]

### Method-BA

Normal phase flash column chromatography [silica gel (120 g), eluting with EtOAc in n-heptane from 0% to 8%]

### Method-BB

Normal phase flash column chromatography [silica gel (40 g), eluting with EtOAc in hexane from 0-50%]

### Method-BC

Normal phase flash column chromatography [silica gel (40 g), eluting with EtOAc/MeOH (9:1) in hexane from 0-100%]

### Method-BD

Normal Phase flash column chromatography [silica gel (24 g), eluting with EtOAc in hexane from 0-25%]

### Method-BE

Normal phase flash column chromatography [silica gel (12 g), eluting with EtOAc in hexane from 0-20%]

### Method-BF

Normal phase flash column chromatography [silica gel (12 g), eluting with EtOAc in hexane from 0-30%]

### Method-BG

Normal phase flash column chromatography [silica gel (12 g), eluting with EtOAc/MeOH (9:1) in hexane from 0-100%]

### Method-BH

Normal phase flash column chromatography [silica gel (24 g), eluting with EtOAc/MeOH (9:1) in hexane from 0-50%]

### Method-BI

Normal phase flash column chromatography [silica gel (24 g), eluting with DMA-80 in DCM from 0-60%]

### Method-BJ

Normal phase flash column chromatography [silica gel (120 g), eluting with EtOAc in n-heptane from 0% to 30%]

### Method-BK

Normal phase flash column chromatography [silica gel (4 g), eluting with DMA-80 in DCM from 0-70%]

### Method-BL

Normal phase flash column chromatography [silica gel (12 g), eluting with DMA-80 in DCM from 0-30%]

### Method-BM

Normal phase flash column chromatography [silica gel (12 g), eluting with MeOH in DCM from 0-20%]

### Method-BN

Normal phase flash column chromatography [silica gel (80 g), eluting with DMA-80 in DCM from 0-70%]

### Method-BQ

Reverse-phase column chromatography [C-18 column (40 g), eluting with ACN in water (containing 0.1% HCl) from 0-100%]

### Method-BP

Normal phase flash column chromatography [silica gel (12 g), eluting with DMA-80 in DCM from 0-80%]

### Method-BQ

Normal phase flash column chromatography [silica gel (120 g), eluting with EtOAc/MeOH (9:1) in hexane from 0-50%]

### Method-BR

Normal phase flash column chromatography [silica gel (40 g), eluting with EtOAc in hexane from 0-30%]

### Method-BS

Normal phase flash column chromatography [silica gel (24 g), eluting with DCM in hexane from 0-50%]

### Method-BT

Normal phase flash column chromatography [silica gel (80 g), eluting with EtOAc in hexane from 0-20%]

### Method-BU

Normal phase flash column chromatography [silica gel (40 g), eluting with MeOH in DCM from 0-10%]

### Method-BV

Reverse-phase column chromatography [C-18 column (50 g), eluting with ACN in water (containing 0.1% HCl) from 0-100%]

### Method-BW

Normal phase flash column chromatography [silica gel (40 g), eluting with DMA-80 in DCM from 0-100%]

### Method-BX

Normal phase flash column chromatography [silica gel (25 g), eluting with EtOAc in hexane from 0-30%]

### Method-BY

Normal phase flash column chromatography [silica gel, eluting with MeOH in DCM from 1-5%]

### Method-BZ

Normal phase flash column chromatography [silica gel, eluting with MeOH in DCM from 1-10%]

### Method-CA

Preparative HPLC purification; Gilson preparative HPLC [Waters X-Select CSH 250 mm×30 mm, 5 m, Mobile phase A: 0.1% TFA in water, Mobile phase B: Acetonitrile, Gradient time/% of B: 0/05, 3/05, 8/15, 15/25, 20/30, 25/35, 26/95, 32/95, 33/05, 40/05. Flow rate: 25 mL/min, Diluent: H₂O+ACN]

### Method-CB

Preparative HPLC purification; Shimadzu preparative HPLC [Waters X-Select CSH 250 mm×30 mm, 5 m, Mobile phase A: 0.1% TFA in water, Mobile phase B: Acetonitrile, Gradient time/% of B: 0/05, 3/05, 8 8/12, 15/18, 20/23, 25/28, 30/35, 35/40, 36/95, 42/95, 43/05, 50/05, Flowrate: 25 mL/min, Diluent: DMSO+THF+Acetonitrile]

### Method-CC

Reverse-phase column chromatography [C-18 column (50 g), eluting with ACN in water (containing 0.1% HCl) from 0-100%]

### Method-CD

Normal phase flash column chromatography [silica gel, eluting with EtOAc in heptane from 5-30%]

### Method-CE

Normal phase flash column chromatography [silica gel, eluting with EtOAc in heptane from 20-80%]

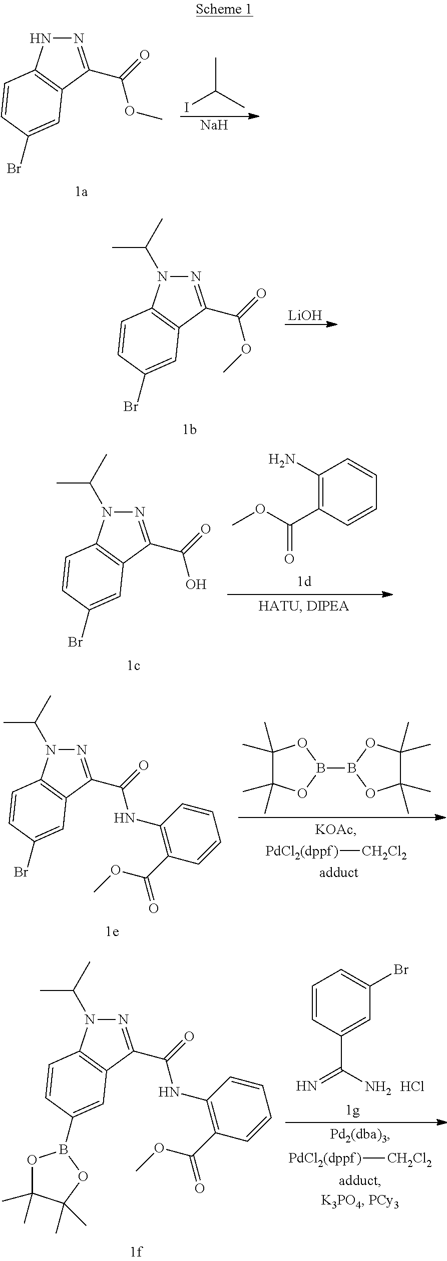

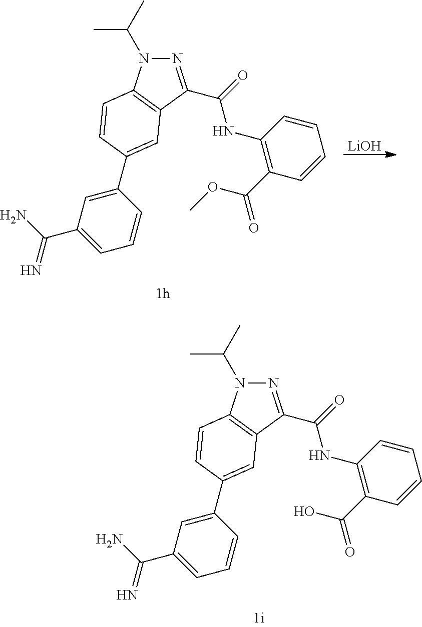

### Preparation of 2-(5-(3-carbamimidoylphenyl)-1-isopropyl-1H-indazole-3-carboxamido)benzoic acid (1i)

### Step-1: Preparation of methyl 5-bromo-1-isopropyl-1H-indazole-3-carboxylate (1b)

To a solution of methyl 5-bromo-1H-indazole-3-carboxylate (1a) (6 g, 23.52 mmol; CAS #78155-74-5) in DMF (50 mL) was added sodium hydride (1.129 g, 28.2 mmol) at 0° C., stirred for 5 mins followed by the addition of 2-iodopropane (7.12 mL, 70.6 mmol) at 0° C. The reaction mixture was allowed to warm to room temperature over a 15 h period and poured into EtOAc. The organic layer was separated, washed with water, brine, dried, filtered and concentrated in vacuum. The residue obtained was purified using method-A, to afford methyl 5-bromo-1-isopropyl-1H-indazole-3-carboxylate (1b) (2.8 g, 40% yield) as a white solid; ¹H NMR (300 MHz, DMSO-d₆) δ 8.20 (dd, J=1.9, 0.7 Hz, 1H), 7.92-7.83 (m, 1H), 7.61 (dd, J=9.0, 1.9 Hz, 1H), 5.21-5.07 (m, 1H), 3.92 (s, 3H), 1.50 (d, J=6.6 Hz, 6H); MS (ES+): 297.20 (M+1).

### Step-2: Preparation of 5-bromo-1-isopropyl-1H-indazole-3-carboxylic acid (1c)

To a solution of methyl 5-bromo-1-isopropyl-1H-indazole-3-carboxylate (1b) (1.4 g, 4.71 mmol) in THE/MeOH (10 mL; 1:1) was added a solution of LiOH·H₂O (0.593 g, 14.13 mmol) in water (3 mL) and stirred at RT for 15 h. The reaction mixture was concentrated in vacuum to dryness and the residue obtained was dissolved in water and acidified to pH˜6. The solid obtained was purified using method-G, to afford 5-bromo-1-isopropyl-1H-indazole-3-carboxylic acid (1c) (1.2 g, 90% yield) as a white solid; ¹H NMR (300 MHz, DMSO-d₆) δ 8.32 (d, J=1.9 Hz, 1H), 7.76 (d, J=8.9 Hz, 1H), 7.52 (dd, J=8.9, 1.9 Hz, 1H), 5.05 (hept, J=6.5 Hz, 1H), 1.48 (d, J=6.6 Hz, 6H); MS (ES+): 283.00 & 285.00 (M+1).

### Step-3: Preparation of methyl 2-(5-bromo-1-isopropyl-1H-indazole-3-carboxamido)benzoate (1e)

To a solution of 5-bromo-1-isopropyl-1H-indazole-3-carboxylic acid (1c) (500 mg, 1.766 mmol) in DMF (10 mL) was added methyl 2-aminobenzoate (1d) (0.251 mL, 1.943 mmol; CAS #134-20-3), N-ethyl-N-isopropylpropan-2-amine (DIPEA, 1.23 mL, 7.06 mmol), 2-(3H-[1,2,3]triazolo[4,5-b]pyridin-3-yl)-1,1,3,3-tetramethylisouronium hexafluorophosphate(V) (HATU, 1007 mg, 2.65 mmol) and the mixture was stirred at RT for 16 h. The reaction mixture was diluted with ethyl acetate, washed with water, brine, dried, filtered and concentrated in vacuum. The residue obtained was purified using method-D, to afford methyl 2-(5-bromo-1-isopropyl-1H-indazole-3-carboxamido)benzoate (1e) (600 mg, 82% yield) as a white solid; 1H NMR (300 MHz, DMSO-d₆) δ 12.27 (s, 1H), 8.82 (dd, J=8.5, 1.2 Hz, 1H), 8.38 (d, J=1.8 Hz, 1H), 8.07 (dd, J=8.0, 1.6 Hz, 1H), 7.92 (d, J=9.0 Hz, 1H), 7.75-7.61 (m, 2H), 7.23 (td, J=7.6, 1.2 Hz, 1H), 5.18 (p. J=6.6 Hz, 1H), 3.95 (s, 3H), 1.59 (d, J=6.6 Hz, 6H); MS (ES+): 416.00 & 418.00 (M+1).

### Step-4: Preparation of methyl 2-(1-isopropyl-5-(4,4,5,5-tetramethyl-1,3,2-dioxaborolan-2-yl)-1H-indazole-3-carboxamido)benzoate (1f)

To a degassed mixture of methyl 2-(5-bromo-1-isopropyl-1H-indazole-3-carboxamido)benzoate (1e) (0.38 g, 0.913 mmol), BISPIN (0.464 g, 1.826 mmol) and potassium acetate (0.224 g, 2.282 mmol) in anhydrous dioxane (10 mL) was added PdCl₂(dppf)-CH₂Cl₂ adduct (0.045 g, 0.055 mmol). The resulting mixture was degassed and filled with argon and heated at 100° C. for 3 b. The reaction mixture was diluted with EtOAc, washed with water (100 mL) and aqueous layer was extracted with EtOAc. The combined organic layers were washed with water, brine, dried, filtered and concentrated in vacuum. The obtained residue was purified using method-B, to give methyl 2-(1-isopropyl-5-(4,4,5,5-tetramethyl-1,3,2-dioxaborolan-2-yl)-1H-indazole-3-carboxamido)benzoate (1f) (0.3 g, 71% yield) as a clear oil; 1H NMR (300 MHz, DMSO-d₆) δ 12.31 (s, 1H), 8.85 (dd, J=8.5, 1.1 Hz, 1H), 8.69 (d, J=1.0 Hz, 1H), 8.08 (dd, J=8.0, 1.6 Hz, 1H), 7.88 (dd, J=8.7, 1.0 Hz, 1H), 7.73 (dt, J=8.3, 1.4 Hz, 2H), 7.24 (td, J=7.7, 1.2 Hz, 1H), 5.19 (p, J=6.6 Hz, 1H), 3.96 (s, 3H), 1.61 (d, J=6.6 Hz, 6H), 1.35 (s, 12H); MS (ES+): 464.20 (M+1); MS (ES+): 464.20 (M+1).

### Step-5: Preparation of methyl 2-(5-(3-carbamimidoylphenyl)-1-isopropyl-1H-indazole-3-carboxamido)benzoate (1h)

To a degassed mixture of methyl 2-(1-isopropyl-5-(4,4,5,5-tetramethyl-1,3,2-dioxaborolan-2-yl)-1H-indazole-3-carboxamido)benzoate (1f) (150 mg, 0.324 mmol) in dioxane/THF (4 mL; ratio 1:1) was added 3-bromobenzimidamide hydrochloride (1g) (152 mg, 0.647 mmol; CAS #16796-52-4), a 2M aqueous solution of tripotassium phosphate (0.647 mL, 1.295 mmol), tricyclohexylphosphine (18.16 mg, 0.065 mmol), Pd₂(dba)₃ (29.6 mg, 0.032 mmol) and PdCl₂(dppf)-CH₂Cl₂ adduct (26.4 mg, 0.032 mmol). The mixture was degassed and filled with Ar and heated at 100° ° C. for 2 h. The mixture was diluted with EtOAc and washed with water and brine. The organic layer was dried, filtered and concentrated in vacuum. The residue obtained was purified using method-E, to give methyl 2-(5-(3-carbamimidoylphenyl)-1-isopropyl-1H-indazole-3-carboxamido)benzoate (1h) (50 mg, 34% yield) as a clear oil; MS (ES+): 456.20 (M+1).

### Step-6: Preparation of 2-(5-(3-carbamimidoylphenyl)-1-isopropyl-1H-indazole-3-carboxamido)benzoic acid (1i)

Compound 1i was prepared according to procedure reported in step-2 of this scheme from methyl 2-(5-(3-carbamimidoylphenyl)-1-isopropyl-1H-indazole-3-carboxamido)benzoate (1h) (50 mg, 0.11 mmol) in THE/MeOH (4 mL; ratio 1:1) using a solution of LiOH·H₂O (27.6 mg, 0.659 mmol) in water (1 mL) to afford after workup and purification using method-G, 2-(5-(3-carbamimidoylphenyl)-1-isopropyl-1H-indazole-3-carboxamido)benzoic acid (1i) (8 mg, 17% yield) HCl salt as a white solid; ¹H NMR (300 MHz, DMSO-d₆) δ 13.80 (s, 1H, D₂O exchangeable), 12.86 (s, 1H, D₂O exchangeable), 9.51 (s, 2H, D₂O exchangeable), 9.11 (s, 2H, D₂O exchangeable), 8.90 (d, J=8.4 Hz, 1H), 8.62 (s, 1H), 8.17 (s, 1H), 8.14-8.01 (m, 3H), 7.95 (d, J=9.0, 1.7 Hz, 1H), 7.82 (d, J=7.6 Hz, 1H), 7.75 (t, J=7.7 Hz, 1H), 7.67 (t, J=8.0 Hz, 1H), 7.20 (t, J=7.6 Hz, 1H), 5.30-5.12 (m, 1H), 1.61 (d, J=6.5 Hz, 6H); MS (ES+): 442.20 (M+1); (ES−): 440.10 (M−1).

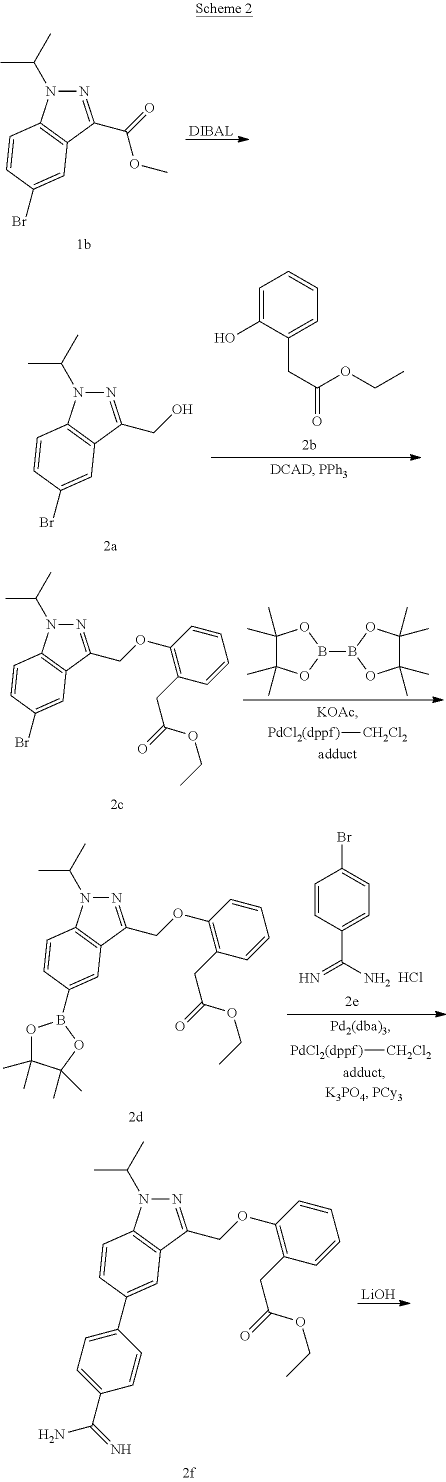

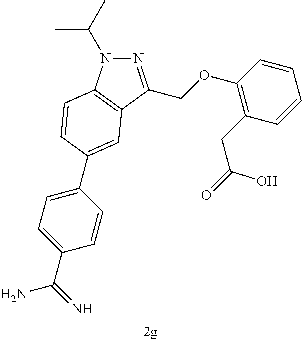 [SMILES: CC(C)n1nc(COc2ccccc2CC(=O)O)c2cc(-c3ccc(C(=N)N)cc3)ccc21]

### Preparation of 2-(2-((5-(4-carbamimidoylphenyl)-1-isopropyl-1H-indazol-3-yl)methoxy)phenyl)acetic acid (2g)

### Step-1: Preparation of (5-bromo-1-isopropyl-1H-indazol-3-yl)methanol (2a)

To a solution of methyl 5-bromo-1-isopropyl-1H-indazole-3-carboxylate (1b) (1.4 g, 4.71 mmol) in THF (20 mL) cooled to −78° C. was added diisobutylaluminum hydride (DIBAL, 1M solution in THF, 11.78 mL, 11.78 mmol) and stirred at −78° C. for 30 min. The reaction was quenched carefully by adding water (15 mL). The mixture was diluted with DCM (100 mL) and stirred vigorously for 1 h. The layers were separated, and the aqueous layer was extracted with DCM (100 mL). The combined organic layers were washed with brine, dried and concentrated in vacuum. The residue obtained was purified using flash column chromatography [silica gel (24 g), eluting with DMA-80 in DCM from 0-100%] to afford (5-bromo-1-isopropyl-1H-indazol-3-yl)methanol (2a) (1.1 g, 87% yield) as a light yellow oil; MS (ES+): 269.00 & 271.00 (M+1).

### Step-2: Preparation of ethyl 2-(2-((5-bromo-1-isopropyl-1H-indazol-3-yl)methoxy)phenyl)acetate (2c)

To a solution of (5-bromo-1-isopropyl-1H-indazol-3-yl)methanol (2a) (550 mg, 2.044 mmol), ethyl 2-(2-hydroxyphenyl)acetate (2b) (387 mg, 2.146 mmol; CAS #41873-65-8) and triphenylphosphine (643 mg, 2.452 mmol) in DCM (10 mL) was added drop wise at 0° C. a solution of (E)-bis(4-chlorobenzyl) diazene-1,2-dicarboxylate (DCAD, 900 mg, 2.452 mmol) in DCM (10 mL). The resulting mixture was stirred for 60 min at room temperature and filtered to remove the insoluble solid. The filtrate was evaporated to dryness and the residue obtained was purified by flash column chromatography [silica gel (24 g), eluting with EtOAc in hexane from 0-60%] to furnish ethyl 2-(2-((5-bromo-1-isopropyl-1H-indazol-3-yl)methoxy)phenyl)acetate (2c) (570 mg, 65% yield) as a white solid; MS (ES+): 431.10 & 433.10 (M+1); MS (ES−): 429.10 & 431.05 (M−1).

### Step-3: Preparation of ethyl 2-(2-((1-isopropyl-5-(4,4,5,5-tetramethyl-1,3,2-dioxaborolan-2-yl)-1H-indazol-3-yl)methoxy)phenyl)acetate (2d)

Compound 2d was prepared according to the procedure reported in step-4 of scheme 1, from ethyl 2-(2-((5-bromo-1-isopropyl-1H-indazol-3-yl)methoxy)phenyl)acetate (2c) (570 mg, 1.322 mmol) in anhydrous dioxane (10 mL) using BISPIN (671 mg, 2.64 mmol), potassium acetate (324 mg, 3.30 mmol), PdCl₂(dppf)-CH₂Cl₂ adduct (64.8 mg, 0.079 mmol) and stirring at 100° C. for 3 h. This gave after work up and purification using flash column chromatography [silica gel (12 g), eluting with EtOAc in hexane from 0-40%] ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 79% yield) as a clear oil; MS (ES+): 479.30 (M+1); 501.30 (M+Na); MS (ES−): 477.15 (M−1).

### Step-4: Preparation of ethyl 2-(2-((5-(4-carbamimidoylphenyl)-1-isopropyl-1H-indazol-3-yl)methoxy)phenyl)acetate (2f)

Compound 2f was prepared according to the procedure reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (250 mg, 0.523 mmol) in dioxane (2 mL) and THF (2 mL) using 4-bromobenzimidamide hydrochloride (2e) (246 mg, 1.045 mmol; CAS #55368-42-8), 2 M solution of K₃PO₄ (1.045 mL, 2.09 mmol), tricyclohexylphosphine (29.3 mg, 0.105 mmol). Pd₂(dba)₃ (47.9 mg, 0.052 mmol), PdCl₂(dppf)-CH₂Cl₂ adduct (42.7 mg, 0.052 mmol) and heating at 100° C. for 2 h. This gave after workup and purification using method-E, ethyl 2-(2-((5-(4-carbamimidoylphenyl)-1-isopropyl-1H-indazol-3-yl)methoxy)phenyl)acetate (21) (110 mg, 45% yield) as a clear oil; MS (ES+): 471.25 (M+1).

### Step-5: Preparation of 2-(2-((5-(4-carbamimidoylphenyl)-1-isopropyl-1H-indazol-3-yl)methoxy)phenyl)acetic acid (2g)

To a solution of ethyl 2-(2-((5-(4-carbamimidoylphenyl)-1-isopropyl-1H-indazol-3-yl)methoxy)phenyl)acetate (2f) (100 mg, 0.213 mmol) in THE/MeOH (4 mL, ratio 1:1) was added a solution of LiOH·H₂O (53.5 mg, 1.275 mmol) in water (1 mL) and stirred at 50° C. for 5 h. This gave after workup as reported in step-2 of scheme 1 and purification using method-G, 2-(2-((5-(4-carbamimidoylphenyl)-1-isopropyl-1H-indazol-3-yl)methoxy)phenyl)acetic acid (2g) (35 mg, 37% yield) HCl salt as a white solid; 1H NMR (300 MHz, DMSO-d₆) δ 9.47-9.32 (m, 2H, D₂O exchangeable), 9.14 (s, 2H, D₂O exchangeable), 8.19 (s, 1H), 8.02-7.91 (m, 4H), 7.89-7.79 (m, 2H), 7.32-7.23 (m, 2H), 7.23-7.16 (m, 1H), 6.95-6.87 (m, 1H), 5.48 (s, 2H), 5.14-4.94 (m, 1H), 3.53 (s, 2H), 1.52 (d, J=6.5 Hz, 6H); MS (ES+): 443.20 (M+1); MS (ES−): 441.20 (M−1).

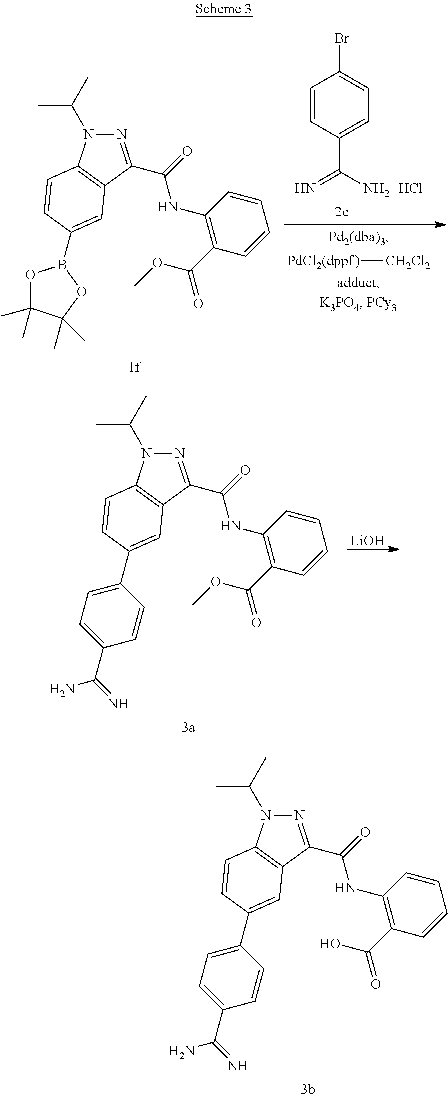

### Preparation of 2-(5-(4-carbamimidoylphenyl)-1-isopropyl-1H-indazole-3-carboxamido)benzoic acid (3b)

### Step-1: Preparation of methyl 2-(5-(4-carbamimidoylphenyl)-1-isopropyl-1H-indazole-3-carboxamido)benzoate (3a)

Compound 3a was prepared according to the procedure reported in step-5 of scheme 1, from methyl 2-(1-isopropyl-5-(4,4,5,5-tetramethyl-1,3,2-dioxaborolan-2-yl)-1H-indazole-3-carboxamido)benzoate (1f) (150 mg, 0.324 mmol) in dioxane (2 mL) and THF (2 mL) using 4-bromobenzimidamide hydrochloride (2e) (152 mg, 0.647 mmol), 2 M solution of K₃PO₄ (0.647 mL, 1.295 mmol), tricyclohexylphosphine (18.16 mg, 0.065 mmol), Pd₂(dba)₃ (29.6 mg, 0.032 mmol), PdCl₂(dppf)-CH₂Cl₂ adduct (26.4 mg, 0.032 mmol) and heating at 100° C. for 2 h. This gave after workup and purification using method-E, methyl 2-(5-(4-carbamimidoylphenyl)-1-isopropyl-1H-indazole-3-carboxamido)benzoate (3a) (35 mg, 24% yield) as a clear oil; MS (ES+): 456.20 (M+1).

### Step-2: Preparation of 2-(5-(4-carbamimidoylphenyl)-1-isopropyl-1H-indazole-3-carboxamido)benzoic acid (3b)

Compound 3b was prepared according to the procedure reported in step-2 of scheme 1, from methyl 2-(5-(4-carbamimidoylphenyl)-1-isopropyl-1H-indazole-3-carboxamido)benzoate (3a) (35 mg, 0.077 mmol) in THF (2 mL) and MeOH (2 mL) using a solution of LiOH·H₂O (19.35 mg, 0.461 mmol) in water (I mL) to afford after workup and purification using method-G, 2-(5-(4-carbamimidoylphenyl)-1-isopropyl-1H-indazole-3-carboxamido)benzoic acid (3b) (12 mg, 35% yield) HCl salt as a white solid; ¹H NMR (300 MHz, DMSO-d₆) δ 12.84 (s, 1H, D₂O exchangeable), 9.39 (s, 2H, D₂O exchangeable), 9.00 (s, 2H, D₂O exchangeable), 8.90 (d, J=8.5 Hz, 1H), 8.58 (s, 1H), 8.12-7.99 (m, 4H), 7.99-7.89 (m, 3H), 7.67 (t, J=7.9 Hz, 1H), 7.20 (t, J=7.6 Hz, 1H), 5.36-5.07 (m, 1H), 1.61 (d, J=6.5 Hz, 6H); MS (ES+): 442.20 (M+1); (ES−): 440.15 (M−1).

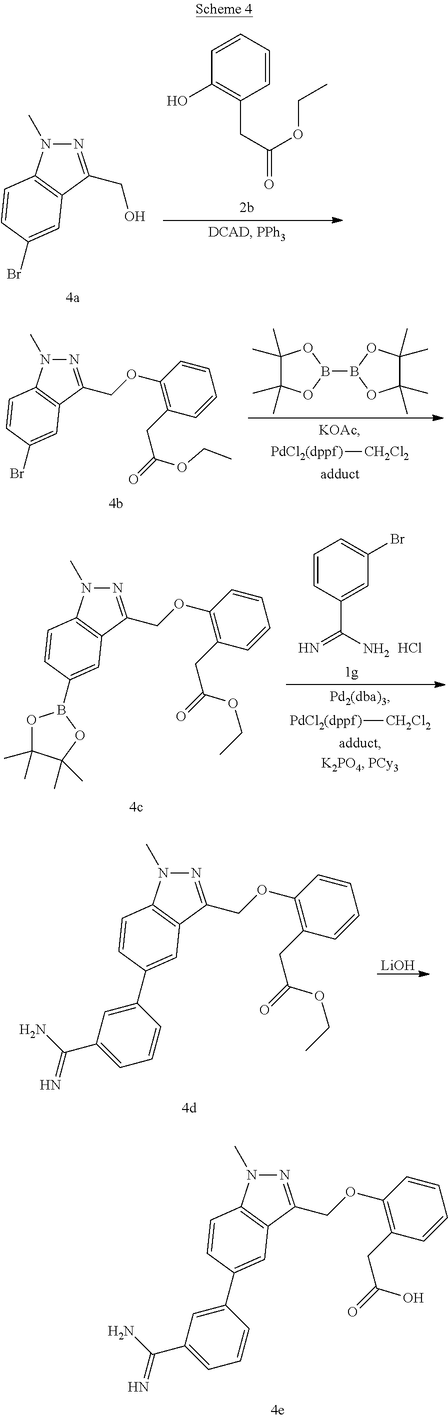

### Preparation of 2-(2-((5-(3-carbamimidoylphenyl)-1-methyl-1H-indazol-3-yl)methoxy)phenyl)acetic acid (4e)

### Step-1: Preparation of ethyl 2-(2-((5-bromo-1-methyl-1H-indazol-3-yl)methoxy)phenyl)acetate (4b)

Compound 4b was prepared according to the procedure reported in step-2 of scheme 2, from (5-bromo-1-methyl-1H-indazol-3-yl)methanol (4a) (500 mg, 2.074 mmol; CAS #1352494-93-9) in DCM (20 mL), using ethyl 2-(2-hydroxyphenyl)acetate (2b) (411 mg, 2.281 mmol), triphenylphosphine (PPh₃) (653 mg, 2.489 mmol), a solution of (E)-bis(4-chlorobenzyl) diazene-1,2-dicarboxylate (DCAD) (914 mg, 2.489 mmol) in DCM (10 mL) to afford after workup using method-H, ethyl 2-(2-((5-bromo-1-methyl-1H-indazol-3-yl)methoxy)phenyl)acetate (4b) (400 mg, 48% yield) as a white solid; MS (ES+): 403.10 & 405.10 (M+1); 425.10 & 427.00 (M+Na); MS (ES−): 401.00 & 403.10 (M−1).

### Step-2: Preparation of ethyl 2-(2-((1-methyl-5-(4,4,5,5-tetramethyl-1,3,2-dioxaborolan-2-yl)-1H-indazol-3-yl)methoxy)phenyl)acetate (4c)

Compound 4c was prepared according to the procedure reported in step-4 of scheme 1, from ethyl 2-(2-((5-bromo-1-methyl-1H-indazol-3-yl)methoxy)phenyl)acetate (4b) (400 mg, 0.992 mmol) in anhydrous dioxane (20 mL), using BISPIN (504 mg, 1.984 mmol), KOAc (243 mg, 2.480 mmol), and PdCl₂(dppf)-CH₂Cl₂ adduct (48.6 mg, 0.06 mmol) to afford after workup and purification using method-C, ethyl 2-(2-((1-methyl-5-(4,4,5,5-tetramethyl-1,3,2-dioxaborolan-2-yl)-1H-indazol-3-yl)methoxy)phenyl)acetate (4c) (320 mg, 72% yield) as a clear oil; 1H NMR (300 MHz, DMSO-d₆) δ 8.17 (d, J=1.0 Hz, 1H), 7.71-7.60 (m, 2H), 7.34-7.24 (m, 2H), 7.20 (d, J=7.3 Hz, 1H), 6.98-6.88 (m, 1H), 5.39 (s, 2H), 4.06 (s, 3H), 3.86 (q, J=7.1 Hz, 2H), 3.50 (s, 2H), 1.31 (s, 12H), 0.89 (t, J=7.1 Hz, 3H); MS (ES+): 451.30 (M+1); 473.20 (M+Na).

### Step-3: Preparation of ethyl 2-(2-((5-(3-carbamimidoylphenyl)-1-methyl-1H-indazol-3-yl)methoxy)phenyl)acetate (4d)

Compound 4d was prepared according to the procedure reported in step-5 of scheme 1, from ethyl 2-(2-((1-methyl-5-(4,4,5,5-tetramethyl-1,3,2-dioxaborolan-2-yl)-1H-indazol-3-yl)methoxy)phenyl)acetate (4c) (320 mg, 0.711 mmol) in dioxane/THF (6 mL, ratio 1:1) using 3-bromobenzimidamide hydrochloride (1g) (335 mg, 1.421 mmol), a 2M solution of K₃PO₄ (1.421 mL, 2.84 mmol), tricyclohexylphosphine (39.9 mg, 0.142 mmol), PdCl₂(dppf)-CH₂Cl₂ adduct (58 mg, 0.071 mmol) and Pd₂(dba)₃ (65.1 mg, 0.071 mmol) to afford after workup and purification using method-F, ethyl 2-(2-((5-(3-carbamimidoylphenyl)-1-methyl-1H-indazol-3-yl)methoxy)phenyl)acetate (4d) (200 mg, 64% yield) as a clear oil; 1H NMR (300 MHz, DMSO-d₆) δ 8.67 (s, 3H), 8.18-8.08 (m, 2H), 8.00 (d, J=7.8 Hz, 1H), 7.87 (d, J=8.8 Hz, 1H), 7.84-7.69 (m, 2H), 7.63 (t, J=7.7 Hz, 1H), 7.28 (q, J=5.7, 4.2 Hz, 2H), 7.19 (d, J=7.4 Hz, 1H), 7.00-6.80 (m, 1H), 5.43 (s, 2H), 4.09 (s, 3H), 3.73-3.62 (m, 2H), 3.52 (s, 2H), 0.87-0.61 (m, 3H); MS (ES+): 443.20 (M+1).

### Step-4: Preparation of 2-(2-((5-(3-carbamimidoylphenyl)-1-methyl-1H-indazol-3-yl)methoxy)phenyl)acetic acid (4e)

Compound 4e was prepared according to the procedure reported in step-5 of scheme 2, from ethyl 2-(2-((5-(3-carbamimidoylphenyl)-1-methyl-1H-indazol-3-yl)methoxy)phenyl)acetate (4d) (200 mg, 0.452 mmol) in THE/MeOH (4 mL, 1:1) using a solution of LiOH·H₂O (114 mg, 2.71 mmol) in water (1 mL) to afford after workup and purification using method-G, 2-(2-((5-(3-carbamimidoylphenyl)-1-methyl-1H-indazol-3-yl)methoxy)phenyl)acetic acid (4e) (80 mg, 43% yield) HCl salt as a white solid; 1H NMR (300 MHz, DMSO-d₆) δ 12.08 (s, 1H, D₂O exchangeable), 9.45 (s, 2H, D₂O exchangeable), 9.18 (s, 2H, D₂O exchangeable), 8.21 (s, 1H), 8.16 (t, 1H), 8.10 (dt, J=7.6, 1.5 Hz, 1H), 7.91 (dd, J=8.8, 1.6 Hz, 1H), 7.85-7.74 (m, 2H), 7.70 (t, J=7.7 Hz, 1H), 7.31-7.23 (m, 2H), 7.23-7.16 (m, 1H), 6.98-6.86 (m, 1H), 5.44 (s, 2H), 4.10 (s, 3H), 3.51 (s, 2H); MS (ES+): 415.15 (M+1); (ES−): 413.15 (M−1).

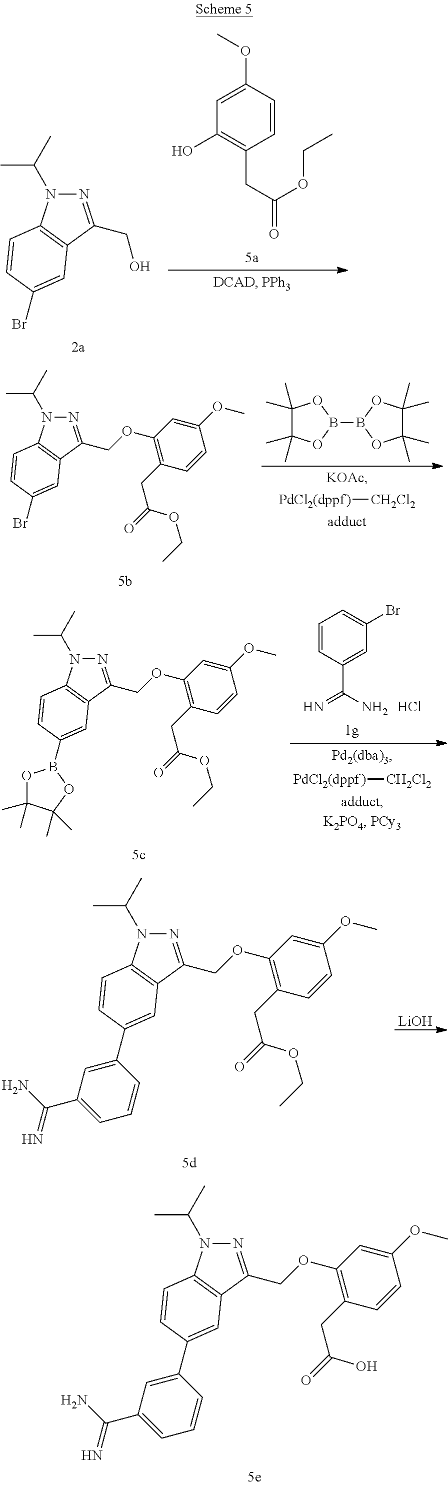

### Preparation of 2-(2-((5-(3-carbamimidoylphenyl)-1-isopropyl-1H-indazol-3-yl)methoxy)-4-methoxyphenyl)acetic acid (5e)

### Step-1: Preparation of ethyl 2-(2-((5-bromo-1-isopropyl-1H-indazol-3-yl)methoxy)-4-methoxyphenyl)acetate (5b)

Compound 5b was prepared according to the procedure reported in step-2 of scheme 2, from (5-bromo-1-isopropyl-1H-indazol-3-yl)methanol (2a) (500 mg, 1.858 mmol) in DCM (20 mL), using ethyl 2-(2-hydroxy-4-methoxyphenyl)acetate (5a) (430 mg, 2.044 mmol; prepared according to the procedure reported by Kotian, Pravin L. et al. PCT Int. Appl. (2019), WO 2019195720 A1 20191010), triphenylphosphine (585 mg, 2.229 mmol), a solution of (E)-bis(4-chlorobenzyl) diazene-1,2-dicarboxylate (DCAD) (819 mg, 2.229 mmol) in DCM (10 mL) to afford after workup and purification using method-H, ethyl 2-(2-((5-bromo-1-isopropyl-1H-indazol-3-yl)methoxy)-4-methoxyphenyl)acetate (5b) (470 mg, 55% yield) as a white solid; MS (ES+): 461.10 & 463.10 (M+1); MS (ES−): 459.00 & 461.05 (M−1).

### Step-2: Preparation of ethyl 2-(2-((1-isopropyl-5-(4,4,5,5-tetramethyl-1,3,2-dioxaborolan-2-yl)-1H-indazol-3-yl)methoxy)-4-methoxyphenyl)acetate (5c)

Compound 5c was prepared according to the procedure reported in step-4 of scheme 1, from ethyl 2-(2-((5-bromo-1-isopropyl-1H-indazol-3-yl)methoxy)-4-methoxyphenyl)acetate (5b) in anhydrous dioxane (20 mL), using BISPIN (517 mg, 2.038 mmol), KOAc (250 mg, 2.55 mmol) and PdCl₂(dppf)-CH₂Cl₂ adduct (49.9 mg, 0.061 mmol) to afford after workup and purification using method-C, ethyl 2-(2-((1-isopropyl-5-(4,4,5,5-tetramethyl-1,3,2-dioxaborolan-2-yl)-1H-indazol-3-yl)methoxy)-4-methoxyphenyl)acetate (5c) (380 mg, 73%) yield) as a clear oil; MS (ES+): 509.30 (M+1).

### Step-3: Preparation of ethyl 2-(2-((5-(3-carbamimidoylphenyl)-1-isopropyl-1H-indazol-3-yl)methoxy)-4-methoxyphenyl)acetate (5d)

Compound 5d was prepared according to the procedure reported in step-5 of scheme 1, from ethyl 2-(2-((1-isopropyl-5-(4,4,5,5-tetramethyl-1,3,2-dioxaborolan-2-yl)-1H-indazol-3-yl)methoxy)-4-methoxyphenyl)acetate (5c) (380 mg, 0.747 mmol) in dioxane/THF (6 mL, 1:1) using 3-bromobenzimidamide hydrochloride (1g) (352 mg, 1.495 mmol), K₃PO₄ (2M aqueous solution, 1.495 mL, 2.99 mmol), PdCl₂(dppf)-CH₂Cl₂ adduct (61 mg, 0.075 mmol), tricyclohexylphosphine (41.9 mg, 0.149 mmol) and Pd₂(dba)₃ (68.4 mg, 0.075 mmol) to afford after workup and purification using method-F, ethyl 2-(2-((5-(3-carbamimidoylphenyl)-1-isopropyl-1H-indazol-3-yl)methoxy)-4-methoxyphenyl)acetate (5d) (200 mg, 54% yield) as a clear oil; 1H NMR (300 MHz, DMSO-d₆) δ 8.36 (s, 2H), 8.08 (s, 2H), 7.93 (d, J=7.7 Hz, 1H), 7.84 (s, 2H), 7.74 (d, J=7.8 Hz, 1H), 7.59 (t, J=7.7 Hz, 1H), 7.07 (d, J=8.3 Hz, 1H), 6.88 (d, J=2.4 Hz, 1H), 6.48 (dd, J=8.3, 2.3 Hz, 1H), 5.44 (s, 2H), 5.06 (p. J=6.5 Hz, 1H), 3.76 (s, 3H), 3.66 (d, J=7.3 Hz, 2H), 3.48 (s, 2H), 3.45 (s, 2H), 1.51 (d, J=6.5 Hz, 6H), 0.78 (t, J=7.1 Hz, 3H); MS (ES+): 501.30 (M+1).

### Step-4: Preparation of 2-(2-((5-(3-carbamimidoylphenyl)-1-isopropyl-1H-indazol-3-yl)methoxy)-4-methoxyphenyl)acetic acid (5e)

Compound 5e was prepared according to the procedure reported in step-6 of scheme 1, from ethyl 2-(2-((5-(3-carbamimidoylphenyl)-1-isopropyl-1H-indazol-3-yl)methoxy)-4-methoxyphenyl)acetate (5d) (200 mg, 0.4 mmol) in THE/MeOH (4 mL, 1:1) using a solution of LiOH·H₂O (101 mg, 2.397 mmol) in water (1 mL) to afford after workup and purification using method-G, 2-(2-((5-(3-carbamimidoylphenyl)-1-isopropyl-1H-indazol-3-yl)methoxy)-4-methoxyphenyl)acetic acid (5e) (35 mg, 19% yield) HCl salt as a white solid; 1H NMR (300 MHz, DMSO-d₆) δ 12.00 (s, 1H, D₂O exchangeable), 9.46 (s, 2H, D₂O exchangeable), 9.19 (s, 2H, D₂O exchangeable), 8.17 (dt, J=9.1, 1.6 Hz, 2H), 8.09 (dt, J=7.6, 1.5 Hz, 1H), 7.87 (s, 2H), 7.81-7.74 (m, 1H), 7.70 (t, J=7.7 Hz, 1H), 7.08 (d, J=8.3 Hz, 1H), 6.89 (d, J=2.4 Hz, 1H), 6.49 (dd, J=8.3, 2.4 Hz, 1H), 5.46 (s, 2H), 5.19-4.94 (m, 1H), 3.75 (s, 3H), 3.44 (s, 2H), 1.52 (d, J=6.5 Hz, 6H); MS (ES+): 473.20 (M+1); MS (ES−): 471.20 (M−1); Analysis calculated for C₂₇H₂₈N₄O₄·HCl·2H₂O: C, 59.50; H, 6.10; Cl, 6.50; N, 10.28; Found: C, 59.32; H, 5.98; Cl, 6.77; N, 10.33.

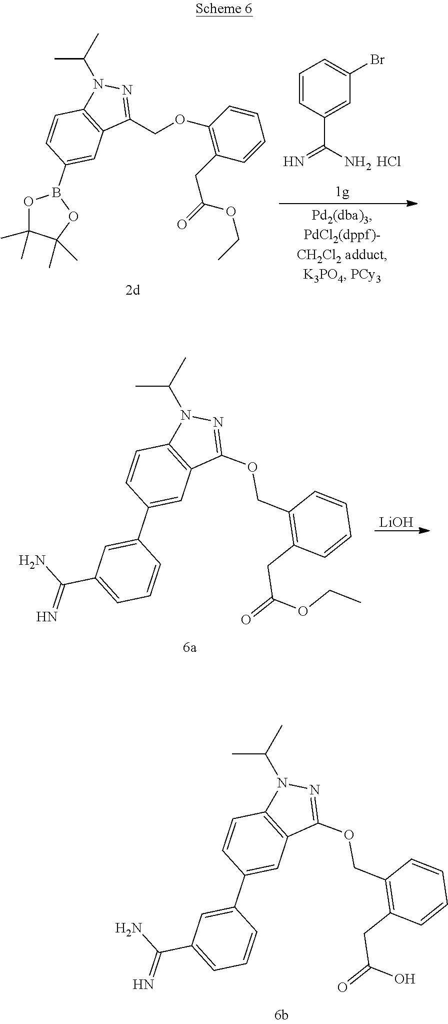

### Preparation of 2-(2-((5-(3-carbamimidoylphenyl)-1-isopropyl-1H-indazol-3-yl)methoxy)phenyl)acetic acid (6b)

### Step-1: Preparation of ethyl 2-(2-((5-(3-carbamimidoylphenyl)-1-isopropyl-1H-indazol-3-yl)methoxy)phenyl)acetate (6a)

Compound 6a was prepared according to the procedure reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (250 mg, 0.523 mmol) in dioxane/THF (4 mL, 1:1) using 3-bromobenzimidamide hydrochloride (1g) (246 mg, 1.045 mmol), K₃PO₄ (2M aqueous solution, 1.045 mL, 2.090 mmol), tricyclohexylphosphine (29.3 mg, 0.105 mmol), PdCl₂(dppf)-CH₂Cl₂ adduct (42.5 mg, 0.052 mmol) and Pd₂(dba)₃ (47.9 mg, 0.052 mmol) to afford after workup and purification using method-E, ethyl 2-(2-((5-(3-carbamimidoylphenyl)-1-isopropyl-1H-indazol-3-yl)methoxy)phenyl)acetate (6a) (70 mg, 29% yield) as a clear oil; MS (ES+): 471.30 (M+1).

### Step-2: Preparation of 2-(2-((5-(3-carbamimidoylphenyl)-I-isopropyl-1H-indazol-3-yl)methoxy)phenyl)acetic acid (6b)

Compound 6b was prepared according to the procedure reported in step-5 of scheme 2, from ethyl 2-(2-((5-(3-carbamimidoylphenyl)-1-isopropyl-1H-indazol-3-yl)methoxy)phenyl)acetate (6a) (70 mg, 0.149 mmol) in THF/MeOH (4 mL, 1:1) using a solution of LiOH·H₂O (37.5 mg, 0.893 mmol) in water (1 mL) to afford after workup and purification using method-G, 2-(2-((5-(3-carbamimidoylphenyl)-1-isopropyl-1H-indazol-3-yl)methoxy)phenyl)acetic acid (6b) (22 mg, 33% yield) HCl salt as a white solid; ¹H NMR (300 MHz, DMSO-d₆) δ 9.46 (s, 2H, D₂O exchangeable), 9.20 (s, 2H, D₂O exchangeable), 8.22-8.13 (m, 2H), 8.09 (d, J=7.8 Hz, 1H), 7.88 (s, 2H), 7.78 (d, J=7.8 Hz, 1H), 7.70 (t, J=7.7 Hz, 1H), 7.31-7.24 (m, 2H), 7.22-7.16 (m, 1H), 6.96-6.87 (m, 1H), 5.46 (s, 2H), 5.11-5.00 (m, 1H), 3.53 (s, 2H), 1.52 (d, J=6.5 Hz, 6H); MS (ES+): 443.20 (M+1); (ES−): 441.20 (M−1).

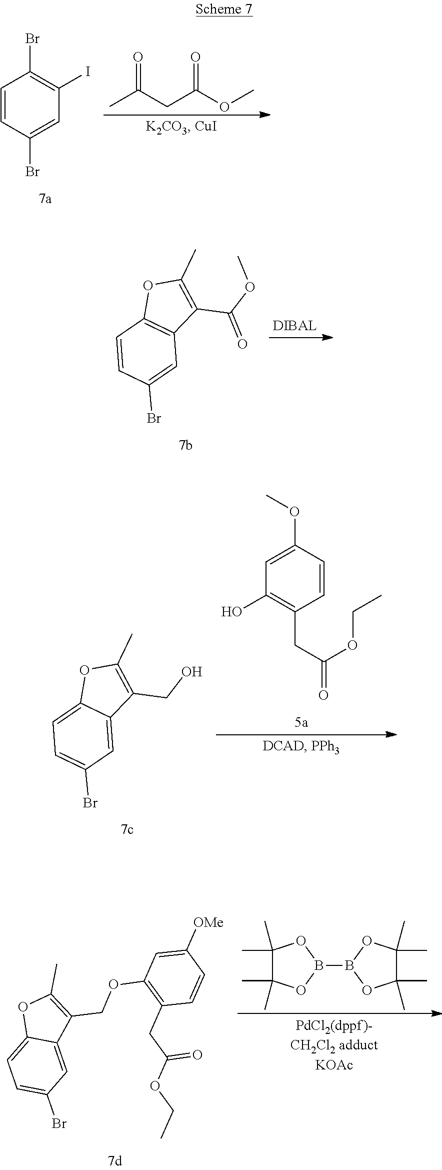

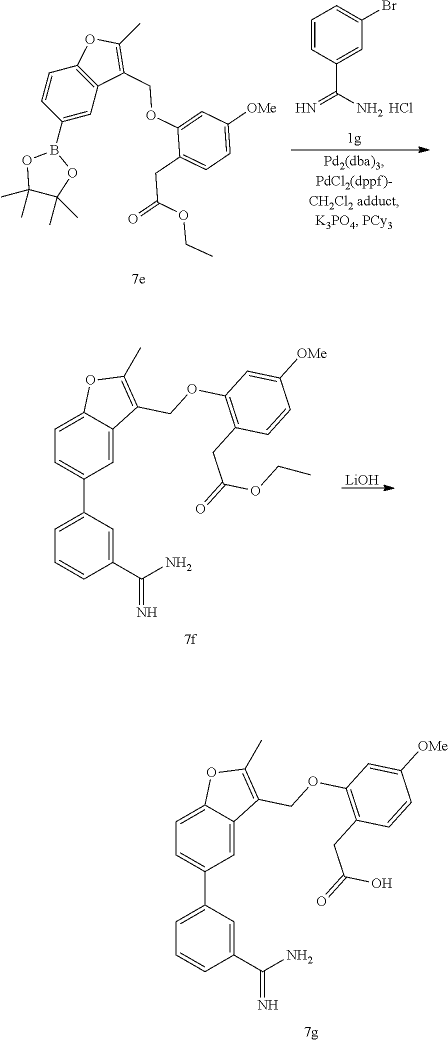

### Preparation of 2-(2-((5-(3-carbamimidoylphenyl)-2-methylbenzofuran-3-yl)methoxy)-4-methoxyphenyl)acetic acid (7g)

### Step-1: Preparation of methyl 5-bromo-2-methylbenzofuran-3-carboxylate (7b)

To a stirred solution of copper(1) iodide (1.369 g, 7.19 mmol) and potassium carbonate (19.10 g, 138 mmol) in THF (50 ml) was added 1,4-dibromo-2-iodobenzene (7a) (20 g, 55.3 mmol; CAS #89284-52-6), methyl acetoacetate (8.95 mL, 83 mmol) under an argon atmosphere and heated at 90° C. for 4 days. The reaction mixture was cooled to room temperature, diluted with EtOAc and extracted with water. The organic layer was dried, filtered and concentrated in vacuum. The obtained residue was purified using method-I to give methyl 5-bromo-2-methylbenzofuran-3-carboxylate (7b) (4.2 g, 28% yield) as a white solid; ¹H NMR (300 MHz, DMSO-d₆) δ 7.96 (d, J=2.1 Hz, 1H), 7.60 (d, J=8.7 Hz, 1H), 7.49 (dd, J=8.7, 2.1 Hz, 1H), 3.89 (s, 3H), 2.73 (s, 3H).

### Step-2: Preparation of (5-bromo-2-methylbenzofuran-3-yl)methanol (7c)

To a solution of methyl 5-bromo-2-methylbenzofuran-3-carboxylate (7b) (2 g, 7.43 mmol) in DCM (20 mL) was added drop wise at 0° ° C. under nitrogen DIBAL (1.0 M in DCM, 18.58 mL, 18.58 mmol) and stirred at 0° C. for 2 h. The reaction mixture was diluted with EtOAc (500 mL) and quenched by successive addition at 0° C. of H₂O (0.74 mL), 15% aqueous NaOH (0.74 mL), and H₂O (1.86 mL). The mixture was then stirred for 15 min at RT, followed by addition of anhydrous MgSO₄ (10 g). After additional 15-min stirring, the cloudy white mixture was filtered. The residue was washed with EtOAc (200 mL). The filtrate was concentrated in vacuum and purified using method-J, to give (5-bromo-2-methylbenzofuran-3-yl)methanol (7c) (1.48 g, 83% yield) as a white solid; 1H NMR (300 MHz, DMSO-d₆) δ 7.79 (d, J=2.0 Hz, 1H), 7.45 (d, J=8.6 Hz, 1H), 7.36 (dd, J=8.6, 2.1 Hz, 1H), 5.06 (t, J=5.6 Hz, 1H), 4.55 (d, J=5.6 Hz, 2H), 2.43 (s, 3H).

### Step-3: Preparation of ethyl 2-(2-((5-bromo-2-methylbenzofuran-3-yl)methoxy)-4-methoxyphenyl)acetate (7d)

Compound 7d was prepared according to the procedure reported in step-2 of scheme 2 from (5-bromo-2-methylbenzofuran-3-yl)methanol (7c) (1 g, 4.15 mmol) in DCM (20 mL), using triphenylphosphine (1.197 g, 4.56 mmol), ethyl 2-(2-hydroxy-4-methoxyphenyl)acetate (5a) (0.959 g, 4.56 mmol), a solution of bis(4-chlorobenzyl) diazene-1,2-dicarboxylate (DCAD) (1.675 g, 4.56 mmol) in DCM (10 mL) to afford after workup and purification using method-K, ethyl 2-(2-((5-bromo-2-methylbenzofuran-3-yl)methoxy)-4-methoxyphenyl)acetate (7d) (875 mg, 49% yield) as a clear oil; 1H NMR (300 MHz, DMSO-d₆) δ 7.75 (d, J=2.0 Hz, 1H), 7.51 (d, J=8.7 Hz, 1H), 7.41 (dd, J=8.7, 2.0 Hz, 1H), 7.09 (d, J=8.3 Hz, 1H), 6.79 (d, J=2.4 Hz, 1H), 6.50 (dd, J=8.3, 2.4 Hz, 1H), 5.18 (s, 2H), 3.91 (q. J=7.1 Hz, 2H), 3.78 (s, 3H), 3.43 (s, 2H), 2.51 (s, 3H), 0.96 (t, J=7.1 Hz, 3H); MS (ES+) 454.6 (M+Na).

### Step-4: Preparation of ethyl 2-(4-methoxy-2-((2-methyl-5-(4,4,5,5-tetramethyl-1,3,2-dioxaborolan-2-yl)benzofuran-3-yl)methoxy)phenyl)acetate (7e)

Compound 7e was prepared according to the procedure reported in step-3 of scheme 2 from ethyl 2-(2-((5-bromo-2-methylbenzofuran-3-yl)methoxy)-4-methoxyphenyl)acetate (7d) (870 mg, 2.008 mmol), in anhydrous dioxane (50 mL), using BISPIN (1020 mg, 4.02 mmol), KOAc (690 mg, 7.03 mmol), PdCl₂(dppf)-CH₂Cl₂ adduct (246 mg, 0.301 mmol) and heating at 6 h at 90° C. A second loading BISPIN (510 mg, 2.008 mmol) and PdCl₂(dppf)-CH₂Cl₂ adduct (164 mg, 0.201 mmol) was added and stirred for overnight for reaction completion. This gave after workup and purification using method-L, ethyl 2-(4-methoxy-2-((2-methyl-5-(4,4,5,5-tetramethyl-1,3,2-dioxaborolan-2-yl)benzofuran-3-yl)methoxy)phenyl)acetate (7e) (750 mg, 78% yield) as a clear oil; 1H NMR (300 MHz, DMSO-d₆) δ 7.94 (s, 1H), 7.58 (dd, J=8.2, 1.2 Hz, 1H), 7.50 (d, J=8.2 Hz, 1H), 7.11-7.05 (m, 1H), 6.83 (d, J=2.3 Hz, 1H), 6.50 (dd, J=8.3, 2.3 Hz, 1H), 5.20 (d, J=8.4 Hz, 2H), 3.85 (q. J=7.1 Hz, 2H), 3.78 (s, 3H), 3.42 (s, 2H), 2.50 (s, 3H), 1.30 (s, 12H), 0.90 (t, J=7.1 Hz, 3H).

### Step-5: Preparation of ethyl 2-(2-((5-(3-carbamimidoylphenyl)-2-methylbenzofuran-3-yl)methoxy)-4-methoxyphenyl)acetate (7f)

Compound 7f was prepared according to the procedure reported in step-5 of scheme 1, from ethyl 2-(4-methoxy-2-((2-methyl-5-(4,4,5,5-tetramethyl-1,3,2-dioxaborolan-2-yl)benzofuran-3-yl)methoxy)phenyl)acetate (7e) (300 mg, 0.625 mmol) in dioxane (20 mL) using 3-bromobenzimidamide (1g) (249 mg, 1.249 mmol). K₃PO₄ (4M aqueous solution, 0.625 mL, 2.498 mmol), tricyclohexylphosphine (52.5 mg, 0.187 mmol), Pd₂(dba)₃ (114 mg, 0.125 mmol) and PdCl₂(dppf)-CH₂Cl₂ adduct (102 mg, 0.125 mmol) to afford after workup and purification using method-E, ethyl 2-(2-((5-(3-carbamimidoylphenyl)-2-methylbenzofuran-3-yl)methoxy)-4-methoxyphenyl)acetate (7f) (97 mg, 0.205 mmol, 32.9% yield) as a clear oil; MS (ES+) 473.2 (M+1).

### Step-6: Preparation of 2-(2-((5-(3-carbamimidoylphenyl)-2-methylbenzofuran-3-yl)methoxy)-4-methoxyphenyl)acetic acid (7g)

Compound 7g was prepared according to procedure reported in step-2 of scheme 1 from ethyl 2-(2-((5-(3-carbamimidoylphenyl)-2-methylbenzofuran-3-yl)methoxy)-4-methoxyphenyl)acetate (7f) (97 mg, 0.205 mmol) in THE (3 mL) using a solution of LiOH·H₂O (105 mg, 2.498 mmol) in water (1 mL) to afford after workup and purification using method-M, 2-(2-((5-(3-carbamimidoylphenyl)-2-methylbenzofuran-3-yl)methoxy)-4-methoxyphenyl)acetic acid (7g) (22 mg, 8% yield) HCl salt as a white solid; ¹H NMR (300 MHz, DMSO-d₆) δ 12.03 (s, 1H, D₂O exchangeable), 9.48 (s, 2H, D₂O exchangeable), 9.20 (s, 2H, D₂O exchangeable), 8.15 (s, 1H), 8.12-8.02 (m, 2H), 7.79 (d, J=7.8 Hz, 1H), 7.76-7.61 (m, 3H), 7.08 (d, J=8.3 Hz, 1H), 6.84 (d, J=2.4 Hz, 1H), 6.50 (dd, J=8.3, 2.4 Hz, 1H), 5.28 (s, 2H), 3.76 (s, 3H), 3.41 (s, 2H), 2.54 (s, 3H); MS (ES+): 445.2 (M+1); (ES−): 443.1 (M−1); Analysis calculated for C₂₆H₂₄N₂O₅·HCl·0.75H₂O: C, 63.16; H, 5.40; N, 5.67; Found: C, 63.02; H, 5.28; N, 5.84.

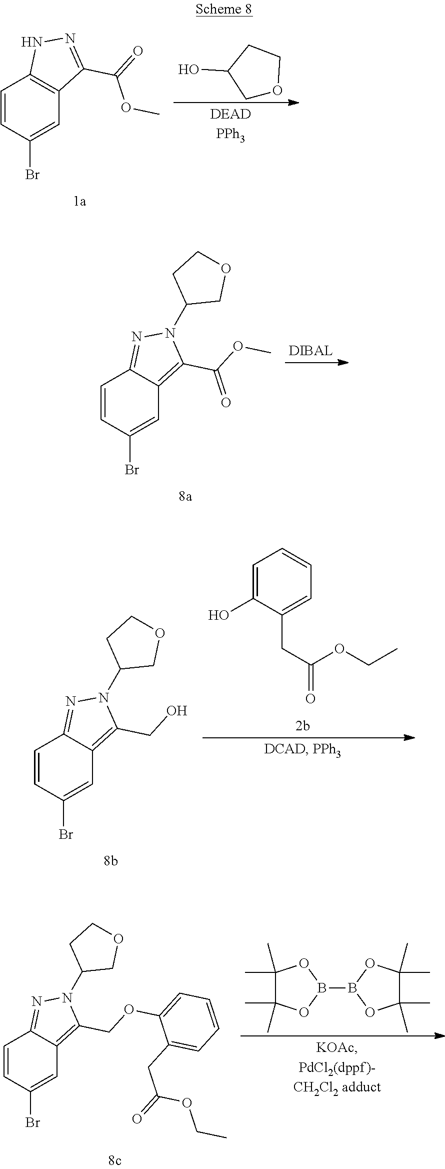

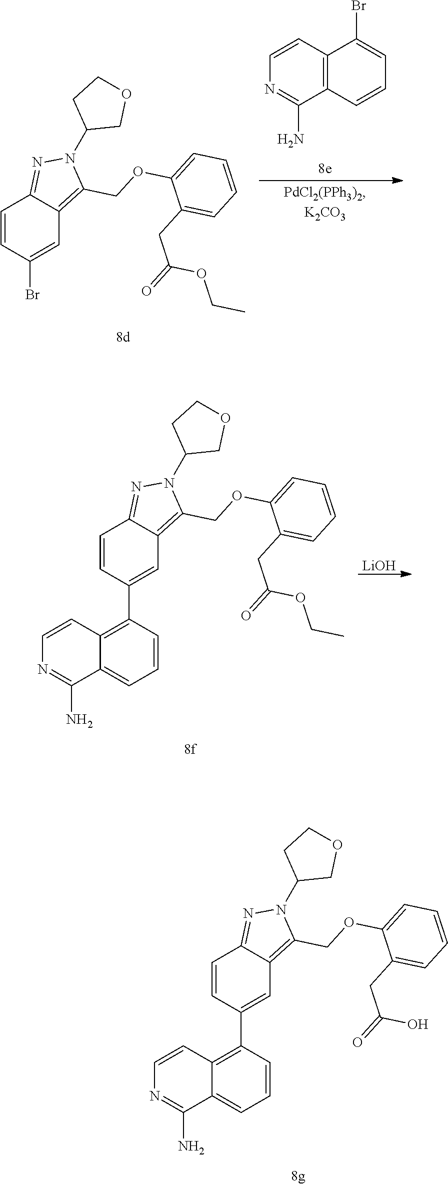 [SMILES: CCOC(=O)Cc1ccccc1OCc1c2cc(Br)ccc2nn1C1CCOC1 ; Nc1nccc2c(Br)cccc12 ; CCOC(=O)Cc1ccccc1OCc1c2cc(-c3cccc4c(N)nccc34)ccc2nn1C1CCOC1 ; "LiOH" ; Nc1nccc2c(-c3ccc4nn(C5CCOC5)c(COc5ccccc5CC(=O)O)c4c3)cccc12]

### Preparation of 2-(2-((5-(1-aminoisoquinolin-5-yl)-2-(tetrahydrofuran-3-yl)-2H-indazol-3-yl)methoxy)phenyl)acetic acid (8g)

### Step-1: Preparation of methyl 5-bromo-2-(tetrahydrofuran-3-yl)-2H-indazole-3-carboxylate (8a)

(E)-diethyl diazene-1,2-dicarboxylate (DEAD) (5.46 g, 31.4 mmol) was added drop wisely to a reaction mixture of triphenylphosphine (8.23 g, 31.4 mmol), tetrahydrofuran-3-ol (2.76 g, 31.4 mmol) and methyl 5-bromo-1H-indazole-3-carboxylate (1a) (4 g, 15.68 mmol) in THF at 0° C., and stirred at 0° C. for 10 min. Solvent was removed under vacuum and the residue obtained was purified using method-N, to give methyl 5-bromo-2-(tetrahydrofuran-3-yl)-2H-indazole-3-carboxylate (8a) (3.6 g, 71% yield) as a white solid; 1H NMR (300 MHz, DMSO-d6) δ 8.17-8.12 (m, 1H), 7.81 (dd, J=9.1, 0.8 Hz, 1H), 7.49 (dd, J=9.1, 1.9 Hz, 1H), 6.22-6.11 (m, 1H), 4.18-4.01 (m, 3H), 3.99 (s, 3H), 3.94-3.86 (m, 1H), 2.58-2.50 (m, 2H).

### Step-2: Preparation of (5-bromo-2-(tetrahydrofuran-3-yl)-2H-indazol-3-yl)methanol (8b)

Compound 8b was prepared according to the procedure reported in step-2 of scheme 7 from methyl 5-bromo-2-(tetrahydrofuran-3-yl)-2H-indazole-3-carboxylate (8a) (3.6 g, 11.07 mmol) in DCM (30 mL) using diisobutylaluminum hydride (1M solution in Dichloromethane, 27.7 mL, 27.7 mmol) to afford after workup and purification using method-O, (5-bromo-2-(tetrahydrofuran-3-yl)-2H-indazol-3-yl)methanol (8b) (2.5 g, 76% yield) as a white solid; 1H NMR (300 MHz, DMSO-d₆) δ 8.04 (d, J=1.9 Hz, 1H), 7.58 (d, J=9.1 Hz, 1H), 7.31 (dd, J=9.1, 1.9 Hz, 1H), 5.50 (td, J=6.4, 5.9, 4.1 Hz, 2H), 4.98-4.89 (m, 2H), 4.21-4.07 (m, 2H), 3.97-3.85 (m, 2H), 2.49-2.38 (m, 2H).

### Step-3: Preparation of ethyl 2-(2-((5-bromo-2-(tetrahydrofuran-3-yl)-2H-indazol-3-yl)methoxy)phenyl)acetate (8c)

Compound 8c was prepared according to the procedure reported in step-2 of scheme 2, from (5-bromo-1-(tetrahydrofuran-3-yl)-2H-indazol-3-yl)methanol (8b) (890 mg, 3.00 mmol) in DCM (20 mL), using PPh₃ (864 mg, 3.29 mmol), ethyl 2-(2-hydroxyphenyl)acetate (2b) (594 mg, 3.29 mmol), and a solution of (E)-bis(4-chlorobenzyl) diazene-1,2-dicarboxylate (DCAD) (1210 mg, 3.29 mmol) in DCM (20 mL) to afford after workup and purification using method-P, ethyl 2-(2-((5-bromo-2-(tetrahydrofuran-3-yl)-2H-indazol-3-yl)methoxy)phenyl)acetate (8c) (480 mg, 35% yield) as a white solid; 1H NMR (300 MHZ, DMSO-d₆) δ 8.14 (d, J=1.9 Hz, 1H), 7.65 (d, J=9.1 Hz, 1H), 7.36 (dd, J=9.2, 1.9 Hz, 2H), 7.32-7.27 (m, 1H), 7.23 (dd, J=7.5, 1.6 Hz, 1H), 6.98 (td, J=7.2, 1.4 Hz, 1H), 5.61 (s, 2H), 5.56-5.46 (m, 1H), 4.21-4.09 (m, 2H), 3.99-3.86 (m, 2H), 3.78 (q. J=7.1 Hz, 2H), 3.52 (s, 2H), 2.48-2.39 (m, 2H), 0.87 (t, J=7.1 Hz, 3H); MS (ES+): 459.1 and 461.1 (M+1); (ES−): 457.1 and 459.0 (M−1).

### Step-4: Preparation of ethyl 2-(2-((2-(tetrahydrofuran-3-yl)-5-(4,4,5,5-tetramethyl-1,3,2-dioxaborolan-2-yl)-2H-indazol-3-yl)methoxy)phenyl)acetate (8d)

Compound 8d was prepared according to the procedure reported in step-4 of scheme 1 from ethyl 2-(2-((5-bromo-2-(tetrahydrofuran-3-yl)-2H-indazol-3-yl)methoxy)phenyl)acetate (8c) (480 mg, 1.045 mmol), in anhydrous dioxane (50 mL), using BISPIN (531 mg, 2.090 mmol), KOAc (256 mg, 2.61 mmol), PdCl₂(dppf)-CH₂Cl₂ adduct (51.2 mg, 0.063 mmol) and heating for 3 h at 100° ° C. This gave after workup and purification using method-Q, ethyl 2-(2-((2-(tetrahydrofuran-3-yl)-5-(4,4,5,5-tetramethyl-1,3,2-dioxaborolan-2-yl)-2H-indazol-3-yl)methoxy)phenyl)acetate (8d) (250 mg, 47% yield) as a clear oil, 1H NMR (300 MHZ, DMSO-d₆) δ 8.24 (t, J=1.0 Hz, 1H), 7.62 (dd, J=8.7, 1.0 Hz, 1H), 7.49 (dd, J=8.7, 1.0 Hz, 1H), 7.36-7.32 (m, 2H), 7.23 (d, J=7.4 Hz, 1H), 7.02-6.94 (m, 1H), 5.65 (s, 2H), 5.57-5.47 (m, 1H), 4.20-4.10 (m, 2H), 3.96-3.93 (m, 2H), 3.73 (q, J=7.1 Hz, 2H), 3.50 (s, 2H), 2.45 (q. J=6.7 Hz, 2H), 1.30 (s, 12H), 0.83 (t, J=7.1 Hz, 3H).

### Step-5: Preparation of ethyl 2-(2-((5-(1-aminoisoquinolin-5-yl)-2-(tetrahydrofuran-3-yl)-2H-indazol-3-yl)methoxy)phenyl)acetate (8f)

To a degassed solution of ethyl 2-(2-((2-(tetrahydrofuran-3-yl)-5-(4,4,5,5-tetramethyl-1,3,2-dioxaborolan-2-yl)-2H-indazol-3-yl)methoxy)phenyl)acetate (8d) (250 mg, 0.494 mmol) in dioxanes (5 mL) was added 5-bromoisoquinolin-1-amine (5e) (165 mg, 0.741 mmol; CAS #852570-80-0), bis(triphenylphosphine)palladium(II) chloride (69.3 mg, 0.099 mmol) and potassium carbonate (205 mg, 1.481 mmol) in water (0.5 mL). The mixture was degassed and filled with Ar and heated at 100° C. for 5 h. The reaction mixture was cooled to RT and diluted with EtOAc, washed with water, brine, dried, filtered and concentrated. The obtained residue was purified using method-C to give ethyl 2-(2-((5-(1-aminoisoquinolin-5-yl)-2-(tetrahydrofuran-3-yl)-2H-indazol-3-yl)methoxy)phenyl)acetate (8f) (96 mg, 37% yield) as a yellow oil; MS (ES+) 523.2 (M+1).

### Step-6: Preparation of 2-(2-((5-(1-aminoisoquinolin-5-yl)-2-(tetrahydrofuran-3-yl)-2H-indazol-3-yl)methoxy)phenyl)acetic acid (8g)

Compound 8g was prepared according to the procedure and method of purification reported in step-2 of scheme 1 from, ethyl 2-(2-((5-(1-aminoisoquinolin-5-yl)-2-(tetrahydrofuran-3-yl)-2H-indazol-3-yl)methoxy)phenyl)acetate (8f) (96 mg, 0.184 mmol) in THF (3 mL) using a solution of LiOH·H₂O (20.72 mg, 0.494 mmol) in water (1 mL) to afford after workup and purification using method-M, 2-(2-((5-(1-aminoisoquinolin-5-yl)-2-(tetrahydrofuran-3-yl)-2H-indazol-3-yl)methoxy)phenyl)acetic acid (8g) (56 mg, 23% yield) HCl salt as a white solid; ¹H NMR (300 MHz, DMSO-d₆) δ 13.53 (s, 1H, D₂O exchangeable), 9.31 (s, 2H, D₂O exchangeable), 8.66 (d, J=8.3 Hz, 1H), 8.02-7.93 (m, 2H), 7.90-7.77 (m, 2H), 7.65 (d, J=7.2 Hz, 1H), 7.36-7.26 (m, 3H), 7.21 (d, J=7.3 Hz, 1H), 7.00-6.91 (m, 2H), 5.66 (d, J=2.1 Hz, 2H), 5.60-5.50 (m, 1H), 4.25-4.14 (m, 2H), 4.03-3.88 (m, 2H), 3.48 (s, 2H), 2.58-2.53 (m, 1H), 2.48-2.37 (m, 1H); MS (ES+): 495.2 (M+1); (ES−): 493.2 (M−1).

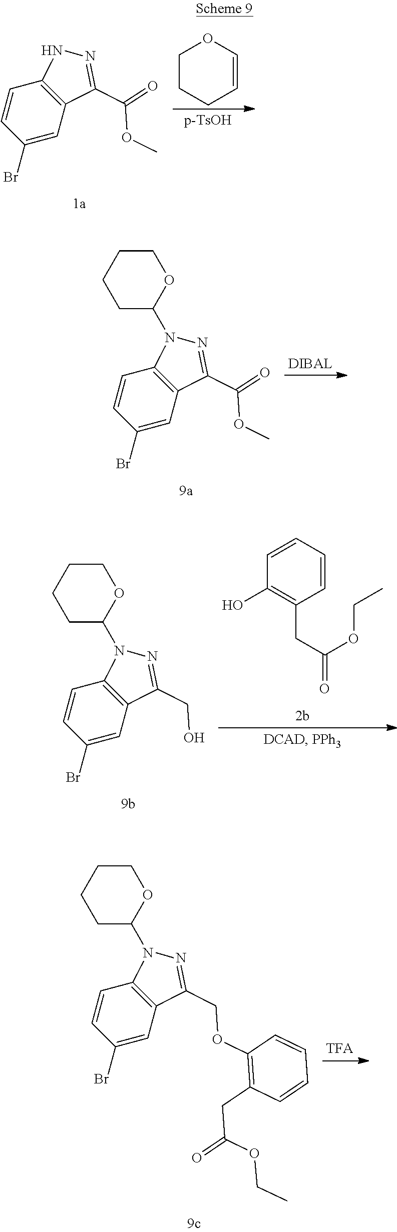

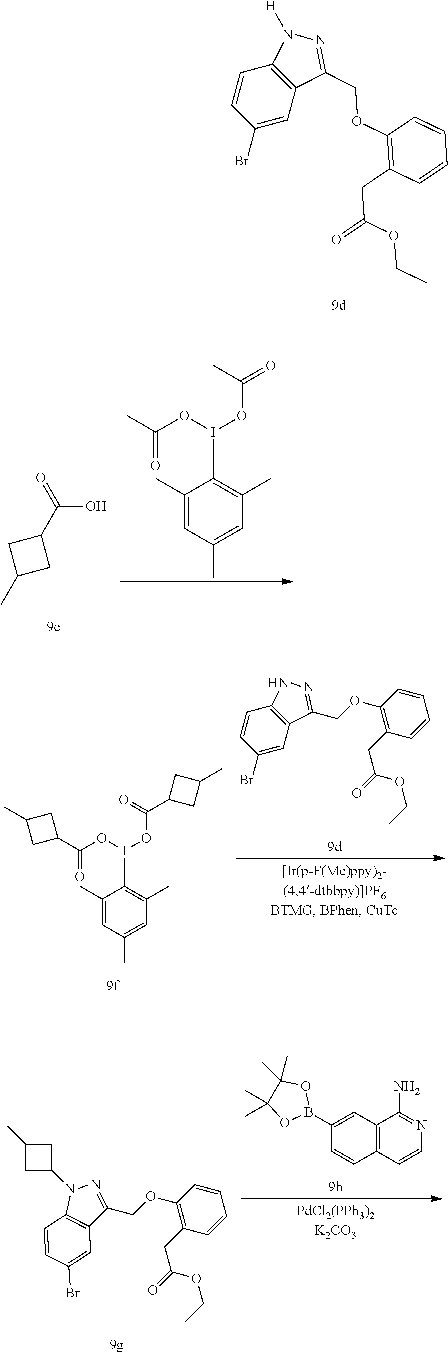

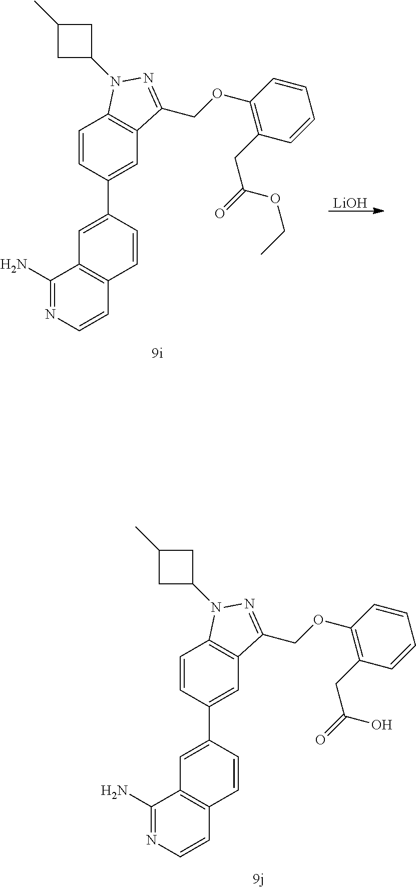 [SMILES: CCOC(=O)Cc1ccccc1OCc1nn(C2CC(C)C2)c2ccc(-c3ccc4ccnc(N)c4c3)cc12 ; "LiOH" ; CC1CC(n2nc(COc3ccccc3CC(=O)O)c3cc(-c4ccc5ccnc(N)c5c4)ccc32)C1]

### Preparation of 2-(2-((5-(1-aminoisoquinolin-7-yl)-1-(3-methylcyclobutyl)-1H-indazol-3-yl)methoxy)phenyl)acetic acid (9))

### Step-1: Preparation of methyl 5-bromo-1-(tetrahydro-2H-pyran-2-yl)-1H-indazole-3-carboxylate (9a)

To a solution of methyl 5-bromo-1H-indazole-3-carboxylate (1a) (3 g, 11.76 mmol; CAS #78155-74-5) in DCM (20 mL) and THF (20 mL) was added p-toluenesulphonic acid (p-TsOH) (0.304 g, 1.764 mmol), 3,4-dihydro-2H-pyran (1.610 mL, 17.64 mmol) and stirred for 4 h at room temperature. The solvent was removed, and the residue obtained was dissolved in methylene chloride (100 mL), washed with a sat. aqueous solution of Na₂CO₃, water and brine. The methylene chloride solution was dried, filtered and concentrated. The resulting oil was purified using method-I, to give methyl 5-bromo-1-(tetrahydro-2H-pyran-2-yl)-1H-indazole-3-carboxylate (9a) (3.6 g, 90% yield) as a white solid; ¹H NMR (300 MHz, DMSO-d6) δ 8.22 (d, J=1.9 Hz, 1H), 7.89 (d, J=9.0 Hz, 1H), 7.67 (dd, J=9.0, 1.9 Hz, 1H), 6.02 (dd, J=9.5, 2.4 Hz, 1H), 3.94 (s, 3H), 3.88 (dt, J=12.3, 3.7 Hz, 1H), 3.77 (ddd, J=11.4, 7.5, 5.6 Hz, 1H), 2.35 (tdd, J=13.0, 9.6, 4.6 Hz, 1H), 2.02 (dd, J=9.4, 4.2 Hz, 2H), 1.73 (td, J=14.2, 13.7, 7.4 Hz, 1H), 1.60 (h, J=4.0 Hz, 2H); MS (ES+): 361.00 & 363.00 (M+Na).

### Step-2: Preparation of (5-bromo-1-(tetrahydro-2H-pyran-2-yl)-1H-indazol-3-yl)methanol (9b)

Compound 9b was prepared according to the procedure reported in step-2 of scheme 7, from methyl 5-bromo-1-(tetrahydro-2H-pyran-2-yl)-1H-indazole-3-carboxylate (9a) (0.6 g, 1.769 mmol) using DIBAL (1.0 M in DCM, 4.42 mL, 4.42 mmol) to afford after workup and purification using method-R, (5-bromo-1-(tetrahydro-2H-pyran-2-yl)-1H-indazol-3-yl)methanol (9b) (0.35 g, 64% yield) as a light yellow oil; 1H NMR (300 MHz, DMSO-d₆) δ 8.07 (d, J=1.8 Hz, 1H), 7.69 (d, J=8.9 Hz, 1H), 7.53 (dd, J=8.9, 1.9 Hz, 1H), 5.80 (dd, J=9.8, 2.4 Hz, 1H), 5.39 (t, J=5.8 Hz, 1H), 4.75 (d, J=5.3 Hz, 2H), 3.92-3.81 (m, 1H), 3.81-3.62 (m, 1H), 2.45-2.25 (m, 1H), 2.12-1.86 (m, 2H), 1.84-1.63 (m, 1H), 1.63-1.45 (m, 2H); MS (ES+): 333.00 (M+Na).

### Step-3: Preparation of ethyl 2-(2-((5-bromo-1-(tetrahydro-2H-pyran-2-yl)-1H-indazol-3-yl)methoxy)phenyl)acetate (9c)

Compound 9c was prepared according to the procedure reported in step-2 of scheme 2, from (5-bromo-1-(tetrahydro-2H-pyran-2-yl)-1H-indazol-3-yl)methanol (9b) (2 g, 6.43 mmol) in DCM (40 mL), using triphenylphosphine (3.37 g, 12.85 mmol), ethyl 2-(2-hydroxyphenyl)acetate (2b) (1.39 g, 7.71 mmol) and a solution of bis(4-chlorobenzyl) diazene-1,2-dicarboxylate (DCAD) (4.72 g, 12.85 mmol) in DCM (20 mL) to afford after workup and purification using method-S, ethyl 2-(2-((5-bromo-1-(tetrahydro-2H-pyran-2-yl)-1H-indazol-3-yl)methoxy)phenyl)acetate (9c) (2.24 g, 74% yield) as a white oil; MS (ES+): 473.10 (M+1).

### Step-4: Preparation of ethyl 2-(2-((5-bromo-1H-indazol-3-yl)methoxy)phenyl)acetate (9d)

To a stirred solution of ethyl 2-(2-((5-bromo-1-(tetrahydro-2H-pyran-2-yl)-1H-indazol-3-yl)methoxy)phenyl)acetate (9c) (2 g, 4.23 mmol) in DCM (30 mL) was added 2,2,2-trifluoroacetic acid (TFA) (3.24 mL, 42.3 mmol) and stirred for 2 h at RT. The reaction mixture was concentrated in vacuum to dryness and the residue obtained was purified using method-T, to afford ethyl 2-(2-((5-bromo-1H-indazol-3-yl)methoxy)phenyl)acetate (9d) (1.44 g, 88% yield) as a yellow oil; ¹H NMR (300 MHz, DMSO-d₆) δ 13.29 (s, 1H), 7.95 (d, J=1.7 Hz, 1H), 7.56-7.51 (m, 1H), 7.48 (dd, J=8.8, 1.7 Hz, 1H), 7.31-7.23 (m, 2H), 7.23-7.16 (m, 1H), 6.92 (td, J=7.1, 1.7 Hz, 1H), 5.38 (s, 2H), 3.91 (q, J=7.1 Hz, 2H), 3.53 (s, 2H), 0.95 (t, J=7.1 Hz, 3H); MS (ES+): 389.05 (M+1).

### Step-5: Preparation of iodomesitylene bis(3-methylcyclobutanecarboxylate) (9f)

To a solution of iodomesitylene diacetate (500 mg, 1.373 mmol) in toluene (12 mL) was added 3-methylcyclobutanecarboxylic acid (9e) (329 mg, 2.88 mmol; CAS #57252-83-2) and the solvent was removed on rotavapor at 55° C. The residue was taken up with toluene (10 mL), then concentrated, and the cycle was repeated for 3 times to give iodomesitylene bis(3-methylcyclobutanecarboxylate) (9f) (640 mg, 99% yield), which was used the next step without further purification.

### Step-6: Preparation of ethyl 2-(2-((5-bromo-1-(3-methylcyclobutyl)-1H-indazol-3-yl)methoxy)phenyl)acetate (9g)

To a solution of ethyl 2-(2-((5-bromo-1H-indazol-3-yl)methoxy)phenyl)acetate (9d) (264 mg, 0.677 mmol) in 1,4-dioxane (10 mL) was added iodomesitylene bis(3-methylcyclobutanccarboxylate) (9f) (640 mg, 1.355 mmol), [Ir(p-F(Mc)ppy)₂-(4,4′-dtbbpy)]PF₆ (6.63 mg, 6.77 μmol), 2-tert-butyl-1,1,3,3-tetramethylguanidine (BTMG, 232 mg, 1.355 mmol), 4,7-diphenyl-1,10-phenanthroline (Bphen) (67.6 mg, 0.203 mmol) and copper(I) thiophene-2-carboxylate (CuTC) (25.8 mg, 0.135 mmol). The solution was sonicated for 1-3 min until it became homogeneous. The mixture was stirred in the photoreactor for 30 min. The reaction mixture was removed from the light, cooled to ambient temperature, diluted with water (15 mL) and EtOAc (25 mL). The aqueous layer was separated and extracted with three portions of EtOAc (25 mL×3). The combined organic layers were washed with brine, dried, filtered, and concentrated. The residue obtained was purified using method-O, to give ethyl 2-(2-((5-bromo-1-(3-methylcyclobutyl)-1H-indazol-3-yl)methoxy)phenyl)acetate (9g) (220 mg, 71% yield) as a clear oil; MS (ES+): 457.1 and 459.1 (M+1).

### Step-7: Preparation of ethyl 2-(2-((5-(1-aminoisoquinolin-7-yl)-1-(3-methylcyclobutyl)-1H-indazol-3-yl)methoxy)phenyl)acetate (9i)

Compound 9i was prepared according to the procedure reported in step-5 of scheme 8 from, ethyl 2-(2-((5-bromo-1-(3-methylcyclobutyl)-1H-indazol-3-yl)methoxy)phenyl)acetate (9g) (220 mg, 0.481 mmol) in dioxanes (5 mL) using 7-(4,4,5,5-tetramethyl-1,3,2-dioxaborolan-2-yl)isoquinolin-1-amine (9h) (195 mg, 0.722 mmol; CAS #1210048-27-3), bis(triphenylphosphine)palladium(II) chloride (67.5 mg, 0.096 mmol) and a solution of K₂CO₃ (199 mg, 1.443 mmol) in water (0.5 mL) to afford after workup and purification using method-C, ethyl 2-(2-((5-(1-aminoisoquinolin-7-yl)-1-(3-methylcyclobutyl)-1H-indazol-3-yl)methoxy)phenyl)acetate (9i) (69 mg, 28% yield) as a yellow oil; MS (ES+): 521.2 (M+1).

### Step-8: Preparation of 2-(2-((5-(1-aminoisoquinolin-7-yl)-1-(3-methylcyclobutyl)-1H-indazol-3-yl)methoxy)phenyl)acetic acid (9j)

Compound 9j was prepared according to the procedure reported in step-2 of scheme 1, from ethyl 2-(2-((5-(1-aminoisoquinolin-7-yl)-1-(3-methylcyclobutyl)-1H-indazol-3-yl)methoxy)phenyl)acetate (9i) (69 mg, 0.133 mmol) in THF (3 mL) using a solution of LiOH·H₂O (60.6 mg, 1.443 mmol) in water (I mL) to afford after workup and purification using method-M, 2-(2-((5-(1-aminoisoquinolin-7-yl)-1-(3-methylcyclobutyl)-1H-indazol-3-yl)methoxy)phenyl)acetic acid (9j) (25 mg, 11% yield) HCl salt as a white solid; ¹H NMR (300 MHz, DMSO-d₆) (a mixture of two rotamers) δ 13.09 (s, 1H, D₂O exchangeable), 12.16 (s, 1H, D₂O exchangeable), 9.10 (s, 2H, D₂O exchangeable), 8.94 (s, 1H), 8.44-8.36 (m, 1H), 8.30 (s, 1H), 8.08-7.97 (m, 2H), 7.89 (t, J=8.1 Hz, 1H), 7.69 (d, J=6.9 Hz, 1H), 7.35-7.24 (m, 3H), 7.24-7.16 (m, 1H), 6.98-6.88 (m, 1H), 5.63-5.52 and 5.24-5.11 (2 m, 1H), 5.50 (s, 2H), 3.53 (s, 2H), 2.88-2.74 (m, 1H), 2.74-2.52 (m, 1H), 2.37-2.09 (m, 3H), 1.32 and 1.20 (2d, J=5.2 Hz, 3H); MS (ES+): 493.2 (M+1); (ES−): 491.2 (M−1).

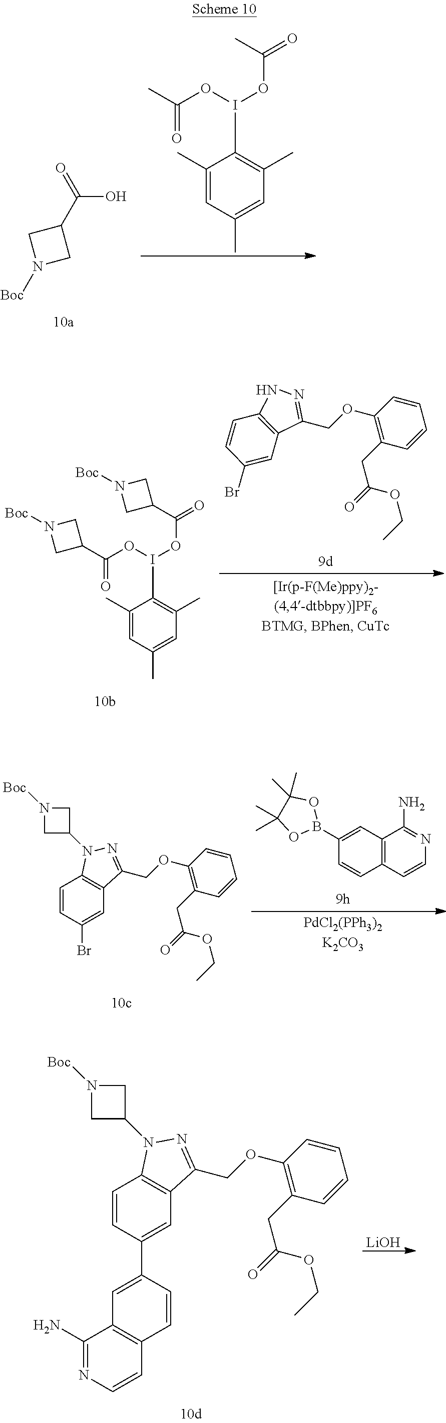

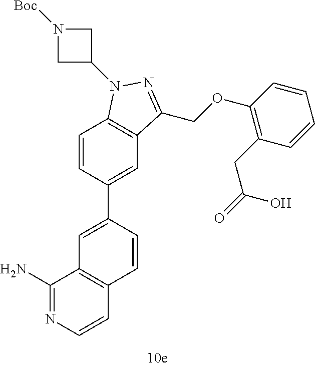 [SMILES: CC(C)(C)OC(=O)N1CC(n2nc(COc3ccccc3CC(=O)O)c3cc(-c4ccc5ccnc(N)c5c4)ccc32)C1]

### Preparation of 2-(2-((5-(1-aminoisoquinolin-7-yl)-1-(1-(tert-butoxycarbonyl)azetidin-3-yl)-1H-indazol-3-yl)methoxy)phenyl)acetic acid (10e)

### Step-1: Preparation of iodomesitylene bis(1-(tert-butoxycarbonyl)azetidine-3-carboxylate) (10b)

Compound 10b was prepared according to the procedure reported in step-5 of scheme 9, from iodomesitylene diacetate (500 mg, 1.373 mmol) in toluene (12 mL) and 1-(tert-butoxycarbonyl)azetidine-3-carboxylic acid (10a) (580 mg, 2.88 mmol, CAS #: 142253-55-2) to afford after workup iodomesitylene bis(1-(tert-butoxycarbonyl)azetidine-3-carboxylate) (10b) (880 mg, 1.361 mmol, 99% yield), which was used in the next step without further purification.

### Step-2: Preparation of tert-butyl 3-(5-bromo-3-((2-(2-ethoxy-2-oxoethyl)phenoxy)methyl)-1H-indazol-1-yl)azetidine-1-carboxylate (10c)

Compound 10c was prepared according to the procedure reported in step-6 of scheme 9, from ethyl 2-(2-((5-bromo-1H-indazol-3-yl)methoxy)phenyl)acetate (9d) (241 mg, 0.619 mmol) in 1,4-dioxane (10 mL) using iodomesitylene bis(1-(tert-butoxycarbonyl)azetidine-3-carboxylate) (10b) (880 mg, 1.361 mmol), [Ir(p-F(Me)ppy)2-(4,4′-dtbbpy)]PF₆ (6.05 mg, 6.19 μmol), 2-tert-butyl-1,1,3,3-tetramethylguanidine (BTMG) (212 mg, 1.237 mmol), 4,7-diphenyl-1,10-phenanthroline (BPhen) (61.7 mg, 0.186 mmol) and copper(I) thiophene-2-carboxylate (CuTC) (23.60 mg, 0.124 mmol) to afford after workup and purification using method-O, tert-butyl 3-(5-bromo-3-((2-(2-ethoxy-2-oxoethyl)phenoxy)methyl)-1H-indazol-1-yl)azetidine-1-carboxylate (10c) (220 mg, 0.404 mmol, 65.3% yield) as a clear oil; MS (ES+): 566.1 and 568.1 (M+Na).

### Step-3: Preparation of tert-butyl 3-(5-(1-aminoisoquinolin-7-yl)-3-((2-(2-ethoxy-2-oxoethyl)phenoxy)methyl)-1H-indazol-1-yl)azetidine-1-carboxylate (10d)

Compound 10d was prepared according to the procedure reported in step-5 of scheme 8, from tert-butyl 3-(5-bromo-3-((2-(2-ethoxy-2-oxoethyl)phenoxy)methyl)-1H-indazol-1-yl)azetidine-1-carboxylate (10c) (220 mg, 0.404 mmol) in Dioxanes (5 mL) using 7-(4,4,5,5-tetramethyl-1,3,2-dioxaborolan-2-yl)isoquinolin-1-amine (9h) (164 mg, 0.606 mmol), PdCl₂(PPh₃)₂ (56.7 mg, 0.081 mmol) and a solution of K₂CO₃ (168 mg, 1.212 mmol) in water (0.5 mL) to afford after workup and purification using method-C, tert-butyl 3-(5-(1-aminoisoquinolin-7-yl)-3-((2-(2-ethoxy-2-oxoethyl)phenoxy)methyl)-1H-indazol-1-yl)azetidine-1-carboxylate (10d) (89 mg, 36.2% yield) as a yellow oil; MS (ES+): 608.3 (M+1).

### Step-4: Preparation of 2-(2-((5-(1-aminoisoquinolin-7-yl)-1-(1-(tert-butoxycarbonyl)azetidin-3-yl)-1H-indazol-3-yl)methoxy)phenyl)acetic acid (10e)

Compound 10e was prepared according to the procedure reported in step-2 of scheme 1, from tert-butyl 3-(5-(1-aminoisoquinolin-7-yl)-3-((2-(2-ethoxy-2-oxoethyl)phenoxy)methyl)-1H-indazol-1-yl)azetidine-1-carboxylate (10d) (89 mg, 0.146 mmol) in THF (3 mL) using a solution of LiOH·H₂O (50.9 mg, 1.212 mmol) in water (1 mL) to afford after workup and purification using method-M, 2-(2-((5-(1-aminoisoquinolin-7-yl)-1-(1-(tert-butoxycarbonyl)azetidin-3-yl)-1H-indazol-3-yl)methoxy)phenyl)acetic acid (10e) (28 mg, 12% yield) HCl salt as a white solid; ¹H NMR (300 MHz, DMSO-d₆) δ 13.11 (s, 1H, D₂O exchangeable), 12.13 (s, 1H, D₂O exchangeable), 9.10 (s, 2H, D₂O exchangeable), 8.96 (s, 1H), 8.47-8.37 (m, 1H), 8.35 (d, J=1.7 Hz, 1H), 8.11-8.01 (m, 2H), 7.89 (d, J=8.9 Hz, 1H), 7.69 (d, J=6.9 Hz, 1H), 7.35-7.25 (m, 3H), 7.22 (d, J=7.4 Hz, 1H), 6.99-6.89 (m, 1H), 5.86-5.72 (m, 1H), 5.54 (s, 2H), 4.44 (t, J=8.5 Hz, 2H), 4.30 (s, 2H), 3.54 (s, 2H), 1.45 (s, 9H); MS (ES+): 580.3 (M+1); (ES−): 578.2 (M−1).

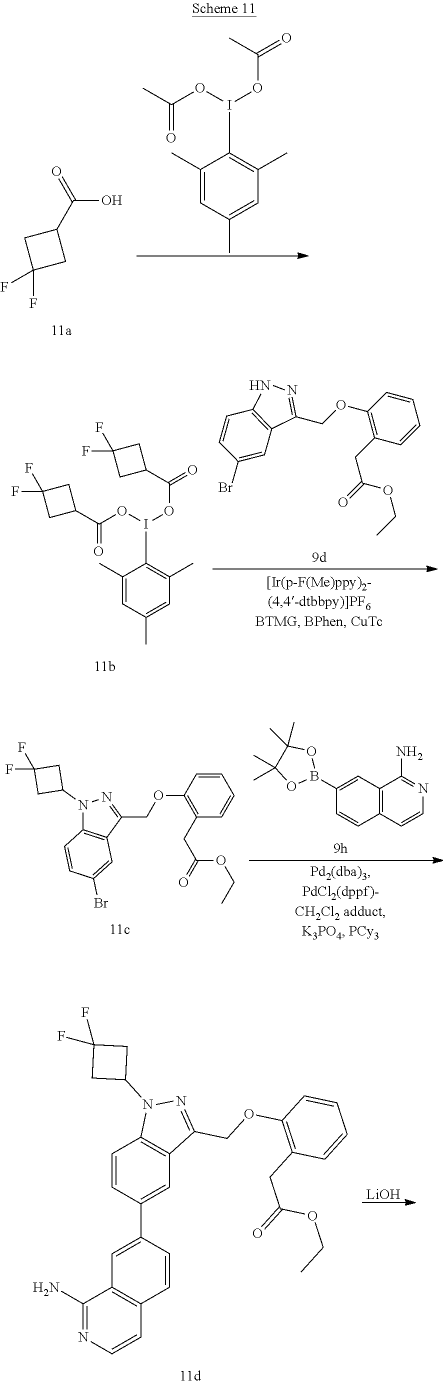

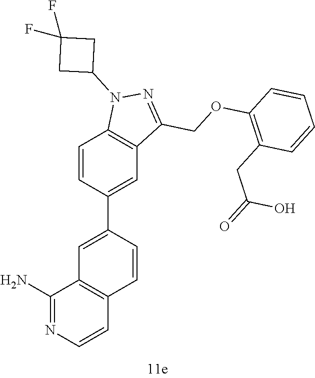 [SMILES: Nc1nccc2ccc(-c3ccc4c(c3)c(COc3ccccc3CC(=O)O)nn4C3CC(F)(F)C3)cc12]

### Preparation of 2-(2-((5-(1-aminoisoquinolin-7-yl)-1-(3,3-difluorocyclobutyl)-1H-indazol-3-yl)methoxy)phenyl)acetic acid (11e)

### Step-1: Preparation of iodomesitylene bis(3,3-difluorocyclobutanecarboxylate) (11b)

Compound 11b was prepared according to the procedure reported in step-5 of scheme 9, from iodomesitylene diacetate (500 mg, 1.373 mmol) in toluene (12 mL) and 3,3-difluorocyclobutanecarboxylic acid (11a) (392 mg, 2.88 mmol), to afford after workup iodomesitylene bis(3,3-difluorocyclobutanecarboxylate) (11b) (705 mg, 99% yield), which was used in the next step without further purification.

### Step-2: Preparation of ethyl 2-(2-((5-bromo-1-(3,3-difluorocyclobutyl)-1H-indazol-3-yl)methoxy)phenyl)acetate (11c)

Compound 11c was prepared according to the procedure reported in step-6 of scheme 9, from ethyl 2-(2-((5-bromo-1H-indazol-3-yl)methoxy)phenyl)acetate (9d) (266 mg, 0.683 mmol) in 1,4-dioxane (10 mL) using iodomesitylene bis(3,3-difluorocyclobutanccarboxylate) (11b) (705 mg, 1.366 mmol), [Ir(p-F(Me)ppy)2-(4,4′-dtbbpy)]PF₆ (6.68 mg, 6.83 μmol), 2-tert-butyl-1,1,3,3-tetramethylguanidine (BTMG) (234 mg, 1.366 mmol), 4,7-diphenyl-1,10-phenanthroline (BPhen) (68.1 mg, 0.205 mmol) and copper(1) thiophene-2-carboxylate (CuTC) (26.0 mg, 0.137 mmol) to afford after workup and purification using method-O, ethyl 2-(2-((5-bromo-1-(3,3-difluorocyclobutyl)-1H-indazol-3-yl)methoxy)phenyl)acetate (11c) (122.9 mg, 37.6% yield) as a clear oil; MS (ES+): 479.1 and 481.1 (M+1).

### Step-3: Preparation of ethyl 2-(2-((5-(1-aminoisoquinolin-7-yl)-1-(3,3-difluorocyclobutyl)-1H-indazol-3-yl)methoxy)phenyl)acetate (11d)

Compound 11d was prepared according to the procedure reported in step-5 of scheme 1, from ethyl 2-(2-((5-bromo-1-(3,3-difluorocyclobutyl)-1H-indazol-3-yl)methoxy)phenyl)acetate (11c) (122 mg, 0.255 mmol) in dioxane/2-MeTHF (4 mL) using 7-(4,4,5,5-tetramethyl-1,3,2-dioxaborolan-2-yl)isoquinolin-1-amine (9h) (103 mg, 0.382 mmol), K₃PO₄ (2M aqueous solution, 0.509 mL, 1.018 mmol), tricyclohexylphosphine (14.28 mg, 0.051 mmol), Pd₂(dba)₃ (23.31 mg, 0.025 mmol), PdCl₂(dppf)-CH₂Cl₂ adduct (20.8 mg, 0.025 mmol) and heating at 100° C. for 5 h on oil bath. This gave after workup and purification using method-U, ethyl 2-(2-((5-(1-aminoisoquinolin-7-yl)-1-(3,3-difluorocyclobutyl)-1H-indazol-3-yl)methoxy)phenyl)acetate (11d) (43 mg, 31% yield) as a clear oil; MS (ES+): 543.2 (M+1).

### Step-4: Preparation of 2-(2-((5-(1-aminoisoquinolin-7-yl)-1-(3,3-difluorocyclobutyl)-1H-indazol-3-yl)methoxy)phenyl)acetic acid (11e)

Compound 11e was prepared according to the procedure reported in step-2 of scheme 1, from ethyl 2-(2-((5-(1-aminoisoquinolin-7-yl)-1-(3,3-difluorocyclobutyl)-1H-indazol-3-yl)methoxy)phenyl)acetate (11d) (43 mg, 0.079 mmol) in THF (3 mL) using a solution of LiOH·H₂O (32.0 mg, 0.764 mmol) in water (1 mL) to afford after workup and purification using method-M, 2-(2-((5-(1-aminoisoquinolin-7-yl)-1-(3,3-difluorocyclobutyl)-1H-indazol-3-yl)methoxy)phenyl)acetic acid (11e) (14 mg, 11% yield) HCl salt as a white solid; 1H NMR (300 MHz, DMSO-d₆) δ 13.02 (s, 1H, D₂O exchangeable), 12.08 (s, 1H, DO exchangeable), 9.04 (s, 2H, D₂O exchangeable), 8.94 (s, 1H), 8.42 (dd, J=8.5, 1.6 Hz, 1H), 8.33 (s, 1H), 8.04 (d, J=8.7 Hz, 2H), 7.97 (d, J=8.8 Hz, 1H), 7.68 (d, J=6.9 Hz, 1H), 7.33-7.23 (m, 3H), 7.23-7.15 (m, 1H), 6.97-6.87 (m, 1H), 5.52 (s, 2H), 5.50-5.41 (m, 1H), 3.53 (s, 2H), 3.32-3.19 (m, 4H); ¹⁹F NMR (282 MHz, DMSO-d₆) δ −81.45, −82.15, −97.33, −98.02; MS (ES+): 515.2 (M+1); (ES−): 513.2 (M−1).

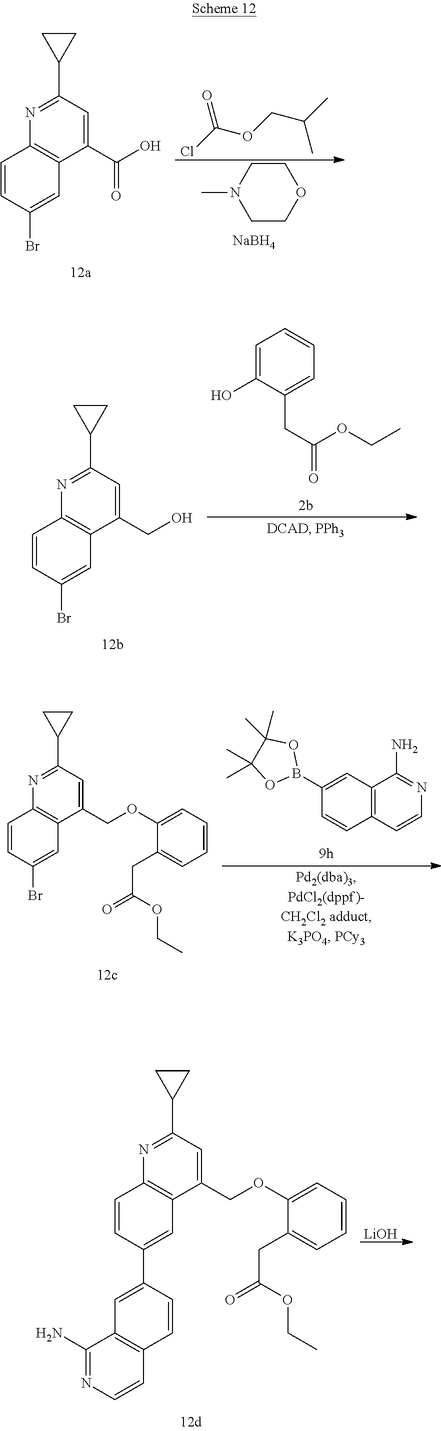

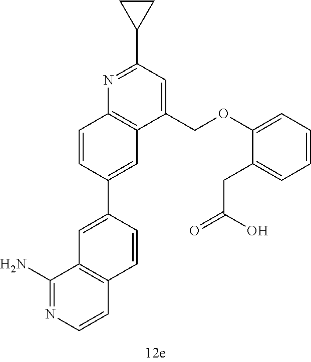 [SMILES: Nc1nccc2ccc(-c3ccc4nc(C5CC5)cc(COc5ccccc5CC(=O)O)c4c3)cc12]

### Preparation of 2-(2-((6-(1-aminoisoquinolin-7-yl)-2-cyclopropylquinolin-4-yl)methoxy)phenyl)acetic acid (12e)

### Step-1: Preparation of (6-bromo-2-cyclopropylquinolin-4-yl)methanol (12b)

To a solution of 6-bromo-2-cyclopropylquinoline-4-carboxylic acid (12a) (800 mg, 2.74 mmol; CAS #313241-16-6) and N-methylmorpholine (0.361 mL, 3.29 mmol) in THF (100 mL) was added isobutyl chloroformate (0.432 mL, 3.29 mmol) at −5° C. After 10 min of stirring the mixture was filtered through a pad of Celite and the precipitate was washed with THF (3×20 mL). The combined organics were cooled to 0° C. and a solution of NaBH₄ (311 mg, 8.22 mmol) in water (5 mL) was added carefully (a gas was released rapidly). The reaction mixture was diluted with water (20 mL), and the solution was washed with EtOAc (3×). The organic layers were collected, dried, filtered and concentrated in vacuum. The residue obtained was purified using method-J to give (6-bromo-2-cyclopropylquinolin-4-yl)methanol (12b) (620 mg, 81% yield) as a white solid; 1H NMR (300 MHz, DMSO-d₆) δ 8.17 (t, J=1.3 Hz, 1H), 7.79 (d, J=1.5 Hz, 2H), 7.49 (s, 1H), 5.59 (t, J=5.5 Hz, 1H), 4.95 (dd, J=5.5, 1.1 Hz, 2H), 2.30 (p, J=6.5 Hz, 1H), 1.13-0.99 (m, 4H).

### Step-2: Preparation of ethyl 2-(2-((6-bromo-2-cyclopropylquinolin-4-yl)methoxy)phenyl)acetate (12c)

Compound 12c was prepared according to the procedure reported in step-2 of scheme 2, from (6-bromo-2-cyclopropylquinolin-4-yl)methanol (12b) (620 mg, 2.229 mmol) in DCM (10 mL), using PPh₃ (1286 mg, 4.90 mmol), ethyl 2-(2-hydroxyphenyl)acetate (2b) (884 mg, 4.90 mmol) and a solution of DCAD (1801 mg, 4.90 mmol) in DCM (20 mL) to afford after workup and purification using method-V, ethyl 2-(2-((6-bromo-2-cyclopropylquinolin-4-yl)methoxy)phenyl)acetate (12c) (450 mg, 46% yield) as a yellow oil; MS (ES+): 440.1 (M+1); (ES−): 442.1 (M−1).

### Step-3: Preparation of ethyl 2-(2-((6-(1-aminoisoquinolin-7-yl)-2-cyclopropylquinolin-4-yl)methoxy)phenyl)acetate (12d)

Compound 12d was prepared according to the procedure reported in step-5 of scheme 1, from ethyl 2-(2-((6-bromo-2-cyclopropylquinolin-4-yl)methoxy)phenyl)acetate (12c) (200 mg, 0.454 mmol) in dioxane/2-MeTHF (4 mL, 3:1) using 7-(4,4,5,5-tetramethyl-1,3,2-dioxaborolan-2-yl)isoquinolin-1-amine (9h) (184 mg, 0.681 mmol), K₃PO₄ (2M aqueous solution, 0.908 mL, 1.817 mmol), tricyclohexylphosphine (25.5 mg, 0.091 mmol), Pd₂(dba)₃ (41.6 mg, 0.045 mmol), PdCl₂(dppf)-CH₂Cl₂ adduct (37.1 mg, 0.045 mmol) to afford after workup and purification using method-U, ethyl 2-(2-((6-(1-aminoisoquinolin-7-yl)-2-cyclopropylquinolin-4-yl)methoxy)phenyl)acetate (12d) (76 mg, 33% yield) as a clear oil; MS (ES+): 504.2 (M+1).

### Step-4: Preparation of 2-(2-((6-(1-aminoisoquinolin-7-yl)-2-cyclopropylquinolin-4-yl)methoxy)phenyl)acetic acid (12e)

Compound 12e was prepared according to the procedure reported in step-2 of scheme 1, from ethyl 2-(2-((6-(1-aminoisoquinolin-7-yl)-2-cyclopropylquinolin-4-yl)methoxy)phenyl)acetate (12d) (76 mg, 0.151 mmol, 33.2% yield) in THF (3 mL) using a solution of LiOH·H₂O (57.2 mg, 1.363 mmol) in water (1 mL) to afford after workup and purification using method-M, 2-(2-((6-(1-aminoisoquinolin-7-yl)-2-cyclopropylquinolin-4-yl)methoxy)phenyl)acetic acid (12e) (32 mg, 15% yield) HCl salt as a white solid; 1H NMR (300 MHz, DMSO-d₆) δ 13.34 (s, 1H, D₂O exchangeable), 9.41 (s, 2H, 1H D₂O exchangeable), 8.77 (s, 1H), 8.62 (dd, J=8.5, 1.6 Hz, 1H), 8.57-8.46 (m, 1H), 8.43-8.28 (m, 1H), 8.14 (d, J=8.5 Hz, 1H), 7.74 (d, J=7.0 Hz, 1H), 7.64 (s, 1H), 7.46 (d, J=8.2 Hz, 1H), 7.36-7.26 (m, 3H), 6.98 (t, J=7.4 Hz, 1H), 6.01 (s, 2H), 3.71 (s, 2H), 2.69-2.54 (m, 1H), 1.50-1.27 (m, 4H); MS (ES+): 476.2 (M+1); (ES−): 474.1 (M−1).

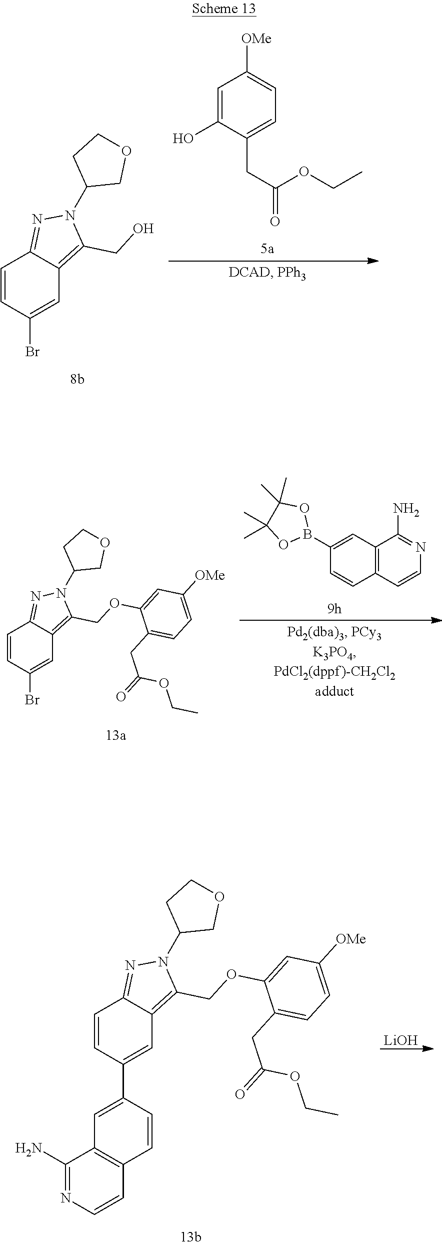 [SMILES: OCc1c2cc(Br)ccc2nn1C1CCOC1 ; CCOC(=O)Cc1ccc(OC)cc1O ; CCOC(=O)Cc1ccc(OC)cc1OCc1c2cc(Br)ccc2nn1C1CCOC1 ; CC1(C)OB(c2ccc3ccnc(N)c3c2)OC1(C)C ; CCOC(=O)Cc1ccc(OC)cc1OCc1c2cc(-c3ccc4ccnc(N)c4c3)ccc2nn1C1CCOC1 ; "LiOH"]

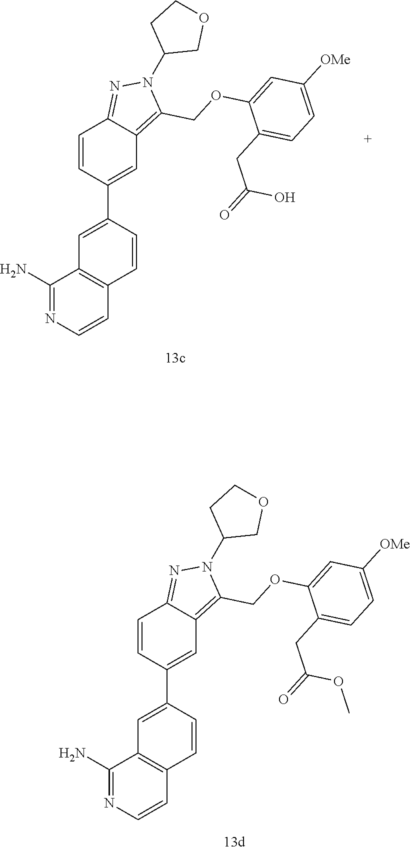 [SMILES: COc1ccc(CC(=O)O)c(OCc2c3cc(-c4ccc5ccnc(N)c5c4)ccc3nn2C2CCOC2)c1 ; COC(=O)Cc1ccc(OC)cc1OCc1c2cc(-c3ccc4ccnc(N)c4c3)ccc2nn1C1CCOC1]

### Preparation of 2-(2-((5-(1-aminoisoquinolin-7-yl)-2-(tetrahydrofuran-3-yl)-2H-indazol-3-yl)methoxy)-4-methoxyphenyl)acetic acid (13c) and methyl 2-(2-((5-(1-aminoisoquinolin-7-yl)-2-(tetrahydrofuran-3-yl)-2H-indazol-3-yl)methoxy)-4-methoxyphenyl)acetate (13d)

### Step-1: Preparation of ethyl 2-(2-((5-bromo-2-(tetrahydrofuran-3-yl)-2H-indazol-3-yl)methoxy)-4-methoxyphenyl)acetate (13a)

Compound 13a was prepared according to the procedure reported in step-2 of scheme 2, from (5-bromo-2-(tetrahydrofuran-3-yl)-2H-indazol-3-yl)methanol (8b) (850 mg, 2.86 mmol) in DCM (20 mL), using PPh₃ (825 mg, 3.15 mmol), ethyl 2-(2-hydroxy-4-methoxyphenyl)acetate (5a) (661 mg, 3.15 mmol) and a solution of DCAD (1155 mg, 3.15 mmol) in DCM (20 mL) to afford after workup and purification using method-J, ethyl 2-(2-((5-bromo-2-(tetrahydrofuran-3-yl)-2H-indazol-3-yl)methoxy)-4-methoxyphenyl)acetate (13a) (630 mg, 45% yield) as a white solid; MS (ES+): 489.1 and 491.1 (M+1).

### Step-2: Preparation of ethyl 2-(2-((5-(1-aminoisoquinolin-7-yl)-2-(tetrahydrofuran-3-yl)-2H-indazol-3-yl)methoxy)-4-methoxyphenyl)acetate (13b)

Compound 13b was prepared according to the procedure reported in step-5 of scheme 1, from ethyl 2-(2-((5-bromo-2-(tetrahydrofuran-3-yl)-2H-indazol-3-yl)methoxy)-4-methoxyphenyl)acetate (13a) (200 mg, 0.409 mmol) in dioxane/2-MeTHF (4 mL, 3:1), using 7-(4,4,5,5-tetramethyl-1,3,2-dioxaborolan-2-yl)isoquinolin-1-amine (9h) (166 mg, 0.613 mmol), K₃PO₄ (2M aqueous solution, 0.817 mL, 1.635 mmol), PCy₃ (22.92 mg, 0.082 mmol), Pd₂(dba)₃ (37.4 mg, 0.041 mmol), PdCl₂(dppf)-CH₂Cl₂ adduct (33.4 mg, 0.041 mmol) to afford after workup and purification using method-U, ethyl 2-(2-((5-(1-aminoisoquinolin-7-yl)-2-(tetrahydrofuran-3-yl)-2H-indazol-3-yl)methoxy)-4-methoxyphenyl)acetate (13b) (42 mg, 19% yield) as a clear oil; MS (ES+): 553.2 (M+1); (ES−): 587.1 (M+Cl).

### Step-3: Preparation of 2-(2-((5-(1-aminoisoquinolin-7-yl)-2-(tetrahydrofuran-3-yl)-2H-indazol-3-yl)methoxy)-4-methoxyphenyl)acetic acid (13c) and methyl 2-(2-((5-(1-aminoisoquinolin-7-yl)-2-(tetrahydrofuran-3-yl)-2H-indazol-3-yl)methoxy)-4-methoxyphenyl)acetate (13d)

Compounds 13c and 13d were prepared according to the procedure reported in step-2 of scheme 1, from ethyl 2-(2-((5-(1-aminoisoquinolin-7-yl)-2-(tetrahydrofuran-3-yl)-2H-indazol-3-yl)methoxy)-4-methoxyphenyl)acetate (13b) (42 mg, 0.076 mmol) in MeOH/THF (3 mL, 1:1) using a solution of LiOH·H₂O (51.5 mg, 1.226 mmol) in water (1 mL) to afford after workup and purification using method-M, 2-(2-((5-(1-aminoisoquinolin-7-yl)-2-(tetrahydrofuran-3-yl)-2H-indazol-3-yl)methoxy)-4-methoxyphenyl)acetic acid (13c) (4 mg, 1.9% yield) HCl salt as a white solid; 1H NMR (300 MHz, DMSO-d₆) δ 13.09 (s, 1H, D₂O exchangeable), 12.19 (s, 1H, D₂O exchangeable), 9.21 (s, 1H, D₂O exchangeable), 8.97 (s, 1H), 8.52-8.40 (m, 2H), 8.06 (d, J=8.6 Hz, 1H), 7.95-7.82 (m, 2H), 7.68 (d, J=6.9 Hz, 1H), 7.28 (d, J=6.9 Hz, 1H), 7.12 (d, J=8.3 Hz, 1H), 6.94 (d, J=2.4 Hz, 1H), 6.54 (dd, J=8.3, 2.4 Hz, 1H), 5.78-5.65 (m, 2H), 5.58-5.44 (m, 1H), 4.24-4.13 (m, 2H), 4.01-3.88 (m, 2H), 3.77 (s, 3H), 3.32 (s, 2H), 2.48-2.34 (m, 2H); MS (ES+): 525.2 (M+1); (ES−): 523.2 (M−1) and methyl 2-(2-((5-(1-aminoisoquinolin-7-yl)-2-(tetrahydrofuran-3-yl)-2H-indazol-3-yl)methoxy)-4-methoxyphenyl)acetate (13d) (11 mg, 5.00% yield) HCl salt as a white solid; ¹H NMR (300 MHz, DMSO-d₆) δ 13.20 (s, 1H, DO exchangeable), 9.21 (s, 1H, D₂O exchangeable), 8.99 (s, 1H), 8.53-8.42 (m, 2H), 8.06 (d, J=8.5 Hz, 1H), 7.97-7.82 (m, 2H), 7.68 (d, J=6.9 Hz, 1H), 7.27 (d, J=7.0 Hz, 1H), 7.12 (d, J=8.3 Hz, 1H), 6.95 (d, J=2.4 Hz, 1H), 6.55 (dd, J=8.3, 2.4 Hz, 1H), 5.72 (s, 2H), 5.57-5.46 (m, 1H), 4.23-4.12 (m, 2H), 4.01-3.88 (m, 2H), 3.78 (s, 3H), 3.48 (s, 2H), 3.32 (s, 3H), 2.48-2.39 (m, 2H); MS (ES+): 539.2 (M+1); (ES−): 537.0 (M−1); 573.2 (M+Cl).

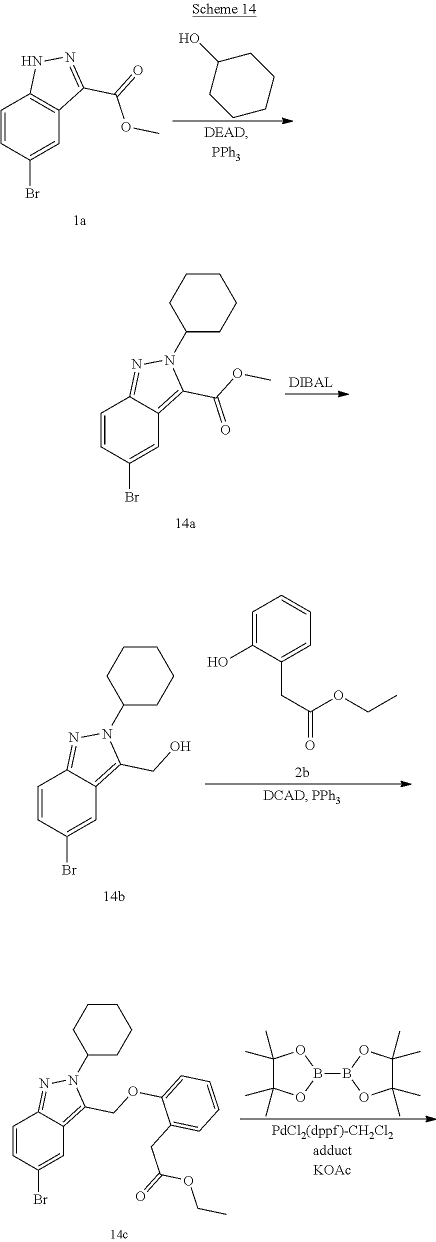

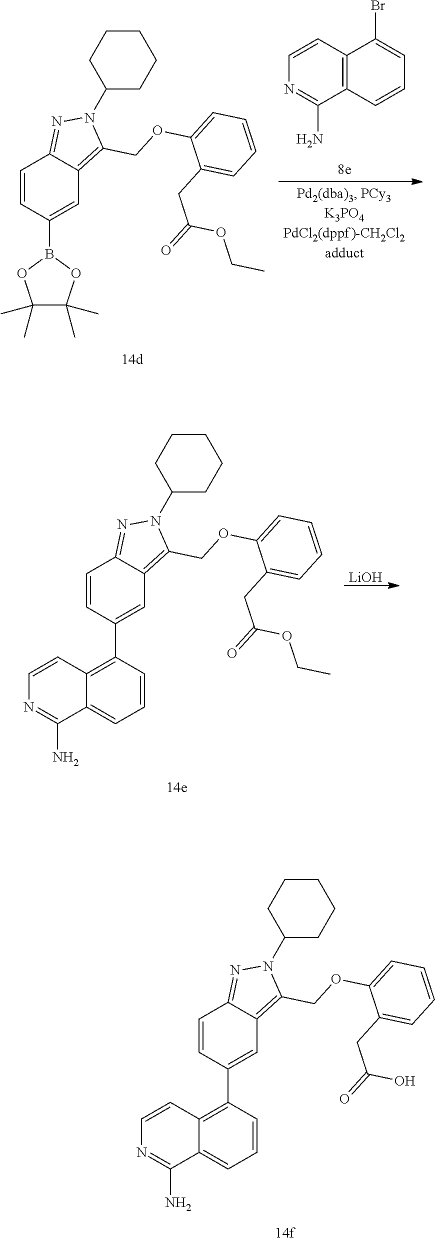

### Preparation of 2-(2-((5-(1-aminoisoquinolin-5-yl)-2-cyclohexyl-2H-indazol-3-yl)methoxy)phenyl)acetic acid (14f)

### Step-1: Preparation of methyl 5-bromo-2-cyclohexyl-2H-indazole-3-carboxylate (14a)

Compound 14a was prepared according to the procedure reported in step-1 of scheme 8, from methyl 5-bromo-1H-indazole-3-carboxylate (1a) (I g, 3.92 mmol) in THE, using DEAD (1.366 g, 7.84 mmol), PPh₃ (2.057 g, 7.84 mmol), and cyclohexanol (0.785 g, 7.84 mmol) to afford after workup and purification using method-N, methyl 5-bromo-2-cyclohexyl-2H-indazole-3-carboxylate (14a) (1.16 g, 3.44 mmol, 88% yield) as a white solid; ¹H NMR (300 MHz, DMSO-d₆) δ 8.14 (d, J=1.9 Hz, 1H), 7.78 (d, J=9.1 Hz, 1H), 7.47 (dd, J=9.1, 1.9 Hz, 1H), 5.44 (tt, J=11.1, 3.9 Hz, 1H), 3.98 (s, 3H), 2.13-1.78 (m, 6H), 1.71 (d, J=12.6 Hz, 1H), 1.52-1.33 (m, 2H), 1.33-1.15 (m, 1H); MS (ES+): 337.05 & 339.10 (M+1).

### Step-2: Preparation of (5-bromo-2-cyclohexyl-2H-indazol-3-yl)methanol (14b)

Compound 14b was prepared according to the procedure reported in step-2 of scheme 7, from methyl 5-bromo-2-cyclohexyl-2H-indazole-3-carboxylate (14a) (4 g, 11.86 mmol) in DCM (50 mL) using diisobutylaluminum hydride (1M solution in Dichloromethane, 29.7 mL, 29.7 mmol) to afford after workup and purification using method-W, (5-bromo-2-cyclohexyl-2H-indazol-3-yl)methanol (14b) (3.0 g, 82% yield) as a white solid; 3H NMR (300 MHz, DMSO-d₆) δ 8.01 (d, J=1.8 Hz, 1H), 7.56 (d, J=9.1 Hz, 1H), 7.28 (dd, J=9.1, 1.9 Hz, 1H), 5.45 (t, J=5.6 Hz, 1H), 4.89 (d, J=5.6 Hz, 2H), 4.66-4.51 (m, 1H), 2.03-1.91 (m, 4H), 1.90-1.78 (m, 2H), 1.76-1.65 (m, 1H), 1.56-1.35 (m, 2H), 1.35-1.15 (m, 1H); MS (ES+): 309.10 & 311.05 (M+1).

### Step-3: Preparation of ethyl 2-(2-((5-bromo-2-cyclohexyl-2H-indazol-3-yl)methoxy)phenyl)acetate (14c)

Compound 14c was prepared according to the procedure reported in step-2 of scheme 2, from (5-bromo-2-cyclohexyl-2H-indazol-3-yl)methanol (14b) (500 mg, 1.617 mmol) in DCM (10 mL), using ethyl 2-(2-hydroxyphenyl)acetate (2b) (306 mg, 1.698 mmol), PPh₃ (509 mg, 1.940 mmol) and a solution of DCAD (713 mg, 1.94 mmol) in DCM (10 mL) to afford after workup and purification using method-X, ethyl 2-(2-((5-bromo-2-cyclohexyl-2H-indazol-3-yl)methoxy)phenyl)acetate (14c) (320 mg, 42.0% yield) as a white oil; 1H NMR (300 MHZ, DMSO-d₆) δ 8.13 (dd, J=2.0, 0.7 Hz, 1H), 7.62 (d, J=9.1 Hz, 1H), 7.34 (dd, J=4.2, 1.8 Hz, 1H), 7.24-7.18 (m, 1H), 7.11-7.02 (m, 1H), 7.00-6.93 (m, 1H), 6.81-6.69 (m, 1H), 5.59 (s, 2H), 4.61 (q, J=7.7 Hz, 1H), 3.81 (q, J=7.1 Hz, 2H), 3.50 (s, 2H), 2.02-1.65 (m, 8H), 1.53-1.40 (m, 2H), 0.89 (t, J=7.1 Hz, 3H); MS (ES+): 471.10 & 473.15 (M+1); MS (ES−): 469.10 & 471.00 (M−1).

### Step-4: Preparation of ethyl 2-(2-((2-cyclohexyl-5-(4,4,5,5-tetramethyl-1,3,2-dioxaborolan-2-yl)-2H-indazol-3-yl)methoxy)phenyl)acetate (14d)

Compound 14d was prepared according to the procedure and method of purification reported in step-4 of scheme 1, from ethyl 2-(2-((5-bromo-2-cyclohexyl-2H-indazol-3-yl)methoxy)phenyl)acetate (14c) (330 mg, 0.7 mmol) in anhydrous dioxane (15 mL), using BISPIN (356 mg, 1.400 mmol), KOAc (172 mg, 1.750 mmol) and PdCl₂(dppf)-CH₂Cl₂ adduct (34.3 mg, 0.042 mmol) to afford after workup and purification using method-C, ethyl 2-(2-((2-cyclohexyl-5-(4,4,5,5-tetramethyl-1,3,2-dioxaborolan-2-yl)-2H-indazol-3-yl)methoxy)phenyl)acetate (14d) (200 mg, 55.1% yield) as a clear oil; 1H NMR (300 MHZ, DMSO-d₆) δ 8.23 (t, J=1.0 Hz, 1H), 7.59 (dd, J=8.7, 1.0 Hz, 1H), 7.46 (dd, J=8.7, 1.0 Hz, 1H), 7.38-7.29 (m, 2H), 7.25-7.16 (m, 1H), 7.02-6.92 (m, 1H), 5.62 (s, 2H), 4.69-4.52 (m, 1H), 3.76 (q, J=7.1 Hz, 2H), 3.48 (s, 2H), 2.11-1.91 (m, 4H), 1.91-1.77 (m, 2H), 1.77-1.64 (m, 1H), 1.53-1.32 (m, 3H), 1.29 (s, 12H), 0.85 (t, J=7.1 Hz, 3H); MS (ES+): 519.30 (M+1).

### Step-5: Preparation of ethyl 2-(2-((5-(1-aminoisoquinolin-5-yl)-2-cyclohexyl-2H-indazol-3-yl)methoxy)phenyl)acetate (14e)

Compound 14e was prepared according to the procedure and method of purification reported in step-5 of scheme 1, from ethyl 2-(2-((2-cyclohexyl-5-(4,4,5,5-tetramethyl-1,3,2-dioxaborolan-2-yl)-2H-indazol-3-yl)methoxy)phenyl)acetate (14d) (200 mg, 0.386 mmol) in dioxane/THF (4 mL, 1:1) using 5-bromoisoquinolin-1-amine (8e) (95 mg, 0.424 mmol), K₃PO₄ (2M aqueous solution, 0.772 mL, 1.543 mmol), PCy₃ (21.64 mg, 0.077 mmol), Pd₂(dba)₃ (35.3 mg, 0.039 mmol) and PdCl₂(dppf)-CH₂Cl₂ adduct (31.5 mg, 0.039 mmol) to afford after workup and purification using method-E, ethyl 2-(2-((5-(1-aminoisoquinolin-5-yl)-2-cyclohexyl-2H-indazol-3-yl)methoxy)phenyl)acetate (14e) (120 mg, 0.224 mmol, 58.2% yield) as a clear oil; 1H NMR (300 MHz, DMSO-d₆) δ 8.21 (d, J=7.5 Hz, 1H), 7.88 (s, 1H), 7.73 (d, J=8.8 Hz, 2H), 7.58-7.50 (m, 2H), 7.35-7.27 (m, 3H), 7.20 (d, J=7.3 Hz, 1H), 7.08-7.00 (m, 1H), 6.94 (t, J=7.0 Hz, 1H), 6.88 (s, 1H), 6.79 (s, 1H), 5.63 (s, 2H), 4.71-4.58 (m, 1H), 3.76 (q, J=7.1 Hz, 2H), 3.51 (s, 2H), 2.06-2.01 (m, 2H), 1.92-1.82 (m, 2H), 1.79-1.66 (m, 2H), 1.57-1.38 (m, 4H), 0.87 (t, J=7.1 Hz, 3H); MS (ES+): 535.30 (M+1).

### Step-6: Preparation of 2-(2-((5-(1-aminoisoquinolin-5-yl)-2-cyclohexyl-2H-indazol-3-yl)methoxy)phenyl)acetic acid (141)

Compound 14f was prepared according to the procedure and method of purification reported in step-2 of scheme 1, from ethyl 2-(2-((5-(1-aminoisoquinolin-5-yl)-2-cyclohexyl-2H-indazol-3-yl)methoxy)phenyl)acetate (14e) (120 mg, 0.224 mmol) in THE/MeOH (4 mL, 1:1) using a solution of LiOH·H₂O (56.5 mg, 1.347 mmol) in water (1 mL) to afford after workup and purification using method-G, 2-(2-((5-(1-aminoisoquinolin-5-yl)-2-cyclohexyl-2H-indazol-3-yl)methoxy)phenyl)acetic acid (14f) (75 mg, 66.0% yield) HCl salt as a white solid; 1H NMR (300 MHz, DMSO-d₆) δ 13.53 (s, 1H, D₂O exchangeable), 9.31 (s, 2H, D₂O exchangeable), 8.66 (d, J=8.3 Hz, 1H), 8.04-7.90 (m, 2H), 7.85 (t, J=7.8 Hz, 1H), 7.79 (d, J=8.9 Hz, 1H), 7.65 (d, J=7.2 Hz, 1H), 7.29 (m, 3H), 7.20 (dd, J=7.4, 1.5 Hz, 1H), 7.02-6.88 (m, 2H), 5.64 (s, 2H), 4.75-4.55 (m, 1H), 3.48 (s, 2H), 2.15-1.94 (m, 4H), 1.94-1.79 (m, 2H), 1.79-1.62 (m, 1H), 1.59-1.37 (m, 2H), 1.37-1.13 (m, 1H); MS (ES+): 507.20 (M+1); MS (ES−): 505.20 (M−1).

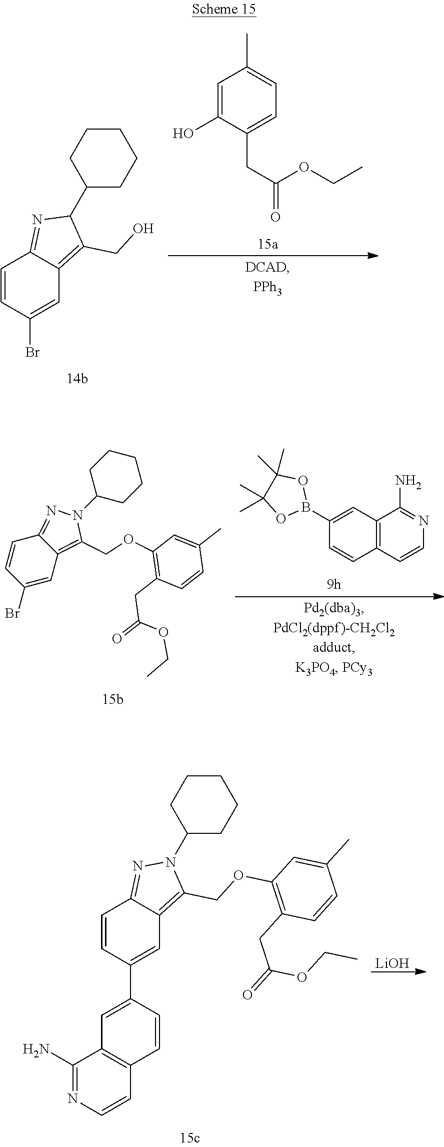

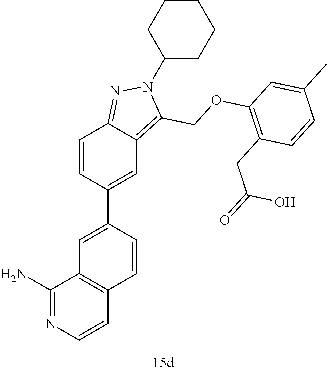 [SMILES: Cc1ccc(CC(=O)O)c(OCc2c3cc(-c4ccc5ccnc(N)c5c4)ccc3nn2C2CCCCC2)c1]

### Preparation of 2-(2-((5-(1-aminoisoquinolin-7-yl)-2-cyclohexyl-2H-indazol-3-yl)methoxy)-4-methylphenyl)acetic acid (15d)

### Step-1: Preparation of ethyl 2-(2-((5-bromo-2-cyclohexyl-2H-indazol-3-yl)methoxy)-4-methylphenyl)acetate (15b)

Compound 15b was prepared according to the procedure reported in step-2 of scheme 2, from (5-bromo-2-cyclohexyl-2H-indazol-3-yl)methanol (14b) (800 mg, 2.59 mmol) in DCM (10 mL), using ethyl 2-(2-hydroxy-4-methylphenyl)acetate (15a) (528 mg, 2.72 mmol), PPh₃ (814 mg, 3.10 mmol) and a solution of DCAD (1140 mg, 3.10 mmol) in DCM (10 mL) to afford after workup and purification using method-N, ethyl 2-(2-((5-bromo-2-cyclohexyl-2H-indazol-3-yl)methoxy)-4-methylphenyl)acetate (15b) (500 mg, 40% yield) as a white oil; 1H NMR (300 MHz, DMSO-d₆) δ 8.15 (dd, J=1.9, 0.7 Hz, 1H), 7.61 (dd, J=9.1, 0.7 Hz, 1H), 7.33 (dd, J=9.1, 1.9 Hz, 1H), 7.16 (d, J=1.6 Hz, 1H), 7.07 (d, J=7.5 Hz, 1H), 6.76 (d, J=7.5 Hz, 1H), 5.57 (s, 2H), 4.59 (p, J=7.9 Hz, 1H), 3.81 (q, J=7.1 Hz, 2H), 3.44 (s, 2H), 2.33 (s, 3H), 2.06-1.61 (m, 8H), 1.54-1.35 (m, 2H), 0.90 (t, J=7.1 Hz, 3H); MS (ES+): 485.10 & 487.10 (M+1); MS (ES−): 483.10 & 485.10 (M−1).

### Step-2: Preparation of ethyl 2-(2-((5-(1-aminoisoquinolin-7-yl)-2-cyclohexyl-2H-indazol-3-yl)methoxy)-4-methylphenyl)acetate (15c)

Compound 15c was prepared according to the procedure reported in step-5 of scheme 1, from ethyl 2-(2-((5-bromo-2-cyclohexyl-2H-indazol-3-yl)methoxy)-4-methylphenyl)acetate (15b) (240 mg, 0.494 mmol) in dioxane/THF (4 mL, 1:1), using 7-(4,4,5,5-tetramethyl-1,3,2-dioxaborolan-2-yl)isoquinolin-1-amine (9h) (200 mg, 0.742 mmol), K₃PO₄ (2M aqueous solution, 0.989 mL, 1.978 mmol), PCy₃ (27.7 mg, 0.099 mmol), PdCl₂(dppf)-CH₂Cl₂ adduct (40.4 mg, 0.049 mmol) and Pd₂(dba)₃ (45.3 mg, 0.049 mmol) to afford after workup and purification using method-Y, ethyl 2-(2-((5-(1-aminoisoquinolin-7-yl)-2-cyclohexyl-2H-indazol-3-yl)methoxy)-4-methylphenyl)acetate (15c) (120 mg, 44% yield) as a clear oil; MS (ES+): 549.30 (M+1).

### Step-3: Preparation of 2-(2-((5-(1-aminoisoquinolin-7-yl)-2-cyclohexyl-2H-indazol-3-yl)methoxy)-4-methylphenyl)acetic acid (15d)

Compound 15d was prepared according to the procedure reported in step-2 of scheme 1, from ethyl 2-(2-((5-(1-aminoisoquinolin-7-yl)-2-cyclohexyl-2H-indazol-3-yl)methoxy)-4-methylphenyl)acetate (15c) (120 mg, 0.219 mmol) in THE/MeOH (4 mL, 1:1) using a solution of LiOH·H₂O (55.1 mg, 1.312 mmol) in water (1 mL)) to afford after workup and purification using method-G, 2-(2-((5-(1-aminoisoquinolin-7-yl)-2-cyclohexyl-2H-indazol-3-yl)methoxy)-4-methylphenyl)acetic acid (15d) (15 mg, 13% yield) HCl salt as a white solid; ¹H NMR (300 MHz, DMSO-d₆) δ 13.20 (s, 1H, DO exchangeable), 9.05 (s, 2H, D₂O exchangeable), 8.98 (s, 1H), 8.53-8.41 (m, 2H), 8.05 (d, J=8.6 Hz, 1H), 7.92-7.78 (m, 2H), 7.68 (d, J=7.0 Hz, 1H), 7.31-7.19 (m, 2H), 7.08 (d, 3=7.5 Hz, 1H), 6.77 (d, J=7.6 Hz, 1H), 5.67 (s, 2H), 4.69-4.50 (m, 1H), 3.52 (s, 2H), 2.33 (s, 3H), 2.07-1.97 (m, 4H), 1.92-1.81 (m, 2H), 1.74-1.66 (m, 1H), 1.50-1.38 (m, 2H), 1.36-1.22 (m, 1H); MS (ES+): 521.30 (M+1); MS (ES−): 519.20 (M−1).

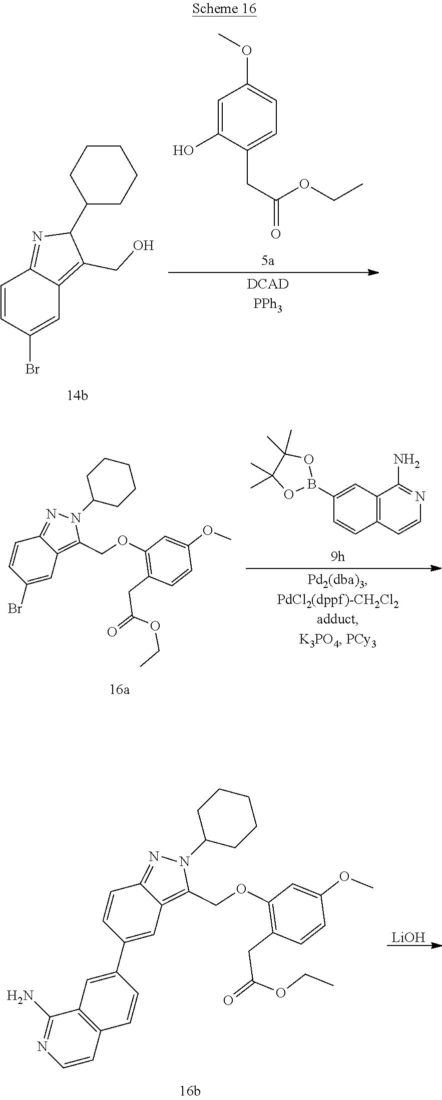 [SMILES: OCC1=c2cc(Br)ccc2=NC1C1CCCCC1 ; CCOC(=O)Cc1ccc(OC)cc1O ; CCOC(=O)Cc1ccc(OC)cc1OCc1c2cc(Br)ccc2nn1C1CCCCC1 ; CC1(C)OB(c2ccc3ccnc(N)c3c2)OC1(C)C ; CCOC(=O)Cc1ccc(OC)cc1OCc1c2cc(-c3ccc4ccnc(N)c4c3)ccc2nn1C1CCCCC1 ; "LiOH"]

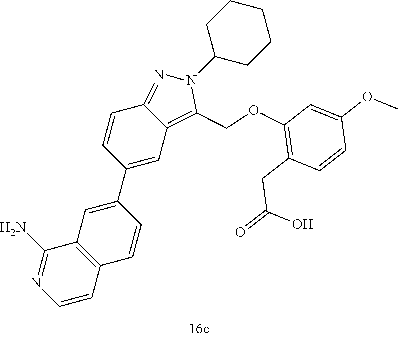 [SMILES: COc1ccc(CC(=O)O)c(OCc2c3cc(-c4ccc5ccnc(N)c5c4)ccc3nn2C2CCCCC2)c1]

### Preparation of 2-(2-((5-(1-aminoisoquinolin-7-yl)-2-cyclohexyl-2H-indazol-3-yl)methoxy)-4-methoxyphenyl)acetic acid (16c)

### Step-1: Preparation of ethyl 2-(2-((5-bromo-2-cyclohexyl-2H-indazol-3-yl)methoxy)-4-methoxyphenyl)acetate (16a)

Compound 16a was prepared according to the procedure and method of purification reported in step-2 of scheme 2, from (5-bromo-2-cyclohexyl-2H-indazol-3-yl)methanol (14b) (800 mg, 2.59 mmol) in DCM (10 mL), using ethyl 2-(2-hydroxy-4-methoxyphenyl)acetate (5a) (571 mg, 2.72 mmol), triphenylphosphine (814 mg, 3.10 mmol) and a solution of DCAD (1140 mg, 3.10 mmol) in DCM (10 mL) to afford after workup and purification using method-N, ethyl 2-(2-((5-bromo-2-cyclohexyl-2H-indazol-3-yl)methoxy)-4-methoxyphenyl)acetate (16a) (440 mg, 0.878 mmol, 33.9% yield) as a white oil; 1H NMR (300 MHz, DMSO-d₆) δ 8.18 (dd, J=1.9, 0.7 Hz, 1H), 7.65-7.56 (m, 1H), 7.36-7.28 (m, 1H), 7.09 (d, J=8.3 Hz, 1H), 6.89 (d, J=2.4 Hz, 1H), 6.52 (dd, J=8.3, 2.4 Hz, 1H), 5.61 (s, 2H), 4.59 (p, J=8.6, 8.0 Hz, 1H), 3.84 (t, J=7.1 Hz, 2H), 3.78 (s, 3H), 3.41 (s, 2H), 2.02-1.68 (m, 8H), 1.53-1.36 (m, 2H), 0.92 (t, J=7.1 Hz, 3H); MS (ES+): 501.10 & 503.10 (M+1); MS (ES−): 499.10 & 501.10 (M−1).

### Step-2: Preparation of ethyl 2-(2-((5-(1-aminoisoquinolin-7-yl)-2-cyclohexyl-2H-indazol-3-yl)methoxy)-4-methoxyphenyl)acetate (16b)

Compound 16b was prepared according to the procedure and method of purification reported in step-5 of scheme 1 from, ethyl 2-(2-((5-bromo-2-cyclohexyl-2H-indazol-3-yl)methoxy)-4-methoxyphenyl)acetate (16a) (220 mg, 0.439 mmol) in dioxane/THF (4 mL, 1:1), using 7-(4,4,5,5-tetramethyl-1,3,2-dioxaborolan-2-yl)isoquinolin-1-amine (9h) (178 mg, 0.658 mmol), K₃PO₄ (2M aqueous solution, 0.878 mL, 1.755 mmol), PCy₃ (24.61 mg, 0.088 mmol), Pd₂(dba)₃ (40.2 mg, 0.044 mmol), PdCl₂(dppf)-CH₂Cl₂ adduct (35.8 mg, 0.044 mmol) and heating at 100° ° C. for 2 h on oil bath. This gave after workup and purification using method-Y, ethyl 2-(2-((5-(1-aminoisoquinolin-7-yl)-2-cyclohexyl-2H-indazol-3-yl)methoxy)-4-methoxyphenyl)acetate (16b) (120 mg, 48.4% yield) as a clear oil; MS (ES+): 565.30 (M+1).

### Step-3: Preparation of 2-(2-((5-(1-aminoisoquinolin-7-yl)-2-cyclohexyl-2H-indazol-3-yl)methoxy)-4-methoxyphenyl)acetic acid (16c)

Compound 16c was prepared according to the procedure and method of purification reported in step-2 of scheme 1, from ethyl 2-(2-((5-(1-aminoisoquinolin-7-yl)-2-cyclohexyl-2H-indazol-3-yl)methoxy)-4-methoxyphenyl)acetate (16b) (120 mg, 0.213 mmol) in THF/MeOH (4 mL, 1:1) using a solution of LiOH·H₂O (53.5 mg, 1.275 mmol) in water (I mL) to afford after workup and purification using method-G, 2-(2-((5-(1-aminoisoquinolin-7-yl)-2-cyclohexyl-2H-indazol-3-yl)methoxy)-4-methoxyphenyl)acetic acid (16c) (18 mg, 0.034 mmol, 15.78% yield) HCl salt as a white solid; 1H NMR (300 MHz, DMSO-d₆) δ 13.29 (s, 1H, D₂O exchangeable), 9.21 (s, 2H, D₂O exchangeable), 9.01 (s, 1H), 8.61-8.51 (m, 1H), 8.45 (dd, J=8.6, 1.6 Hz, 1H), 8.05 (d, J=8.5 Hz, 1H), 7.94-7.78 (m, 2H), 7.68 (d, J=6.9 Hz, 1H), 7.27 (d, J=6.9 Hz, 1H), 7.15-7.06 (m, 1H), 6.96 (d, J=2.4 Hz, 1H), 6.53 (dd, J=8.3, 2.4 Hz, 1H), 5.71 (s, 2H), 4.68-4.52 (m, 1H), 3.76 (s, 3H), 3.40 (s, 2H), 2.10-1.94 (m, 4H), 1.91-1.83 (m, 2H), 1.74-1.67 (m, 1H), 1.51-1.39 (m, 2H), 1.35-1.23 (m, 1H); MS (ES+): 537.30 (M+1); MS (ES−): 535.25 (M−1).

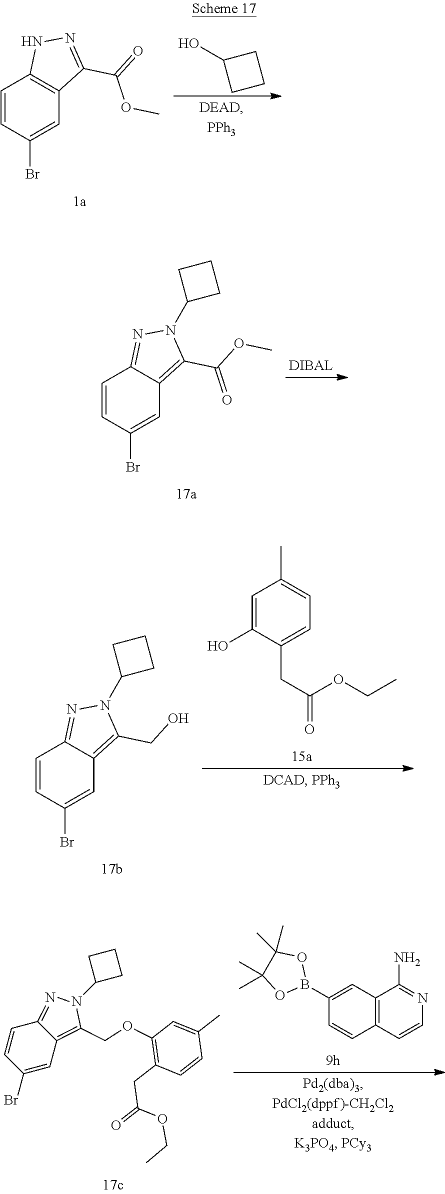

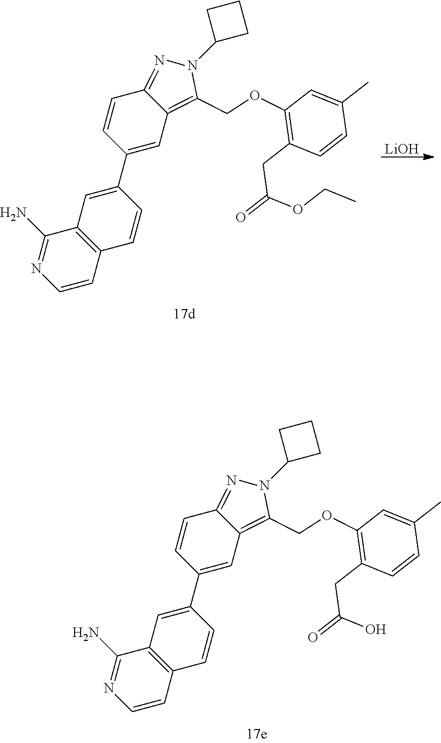

### Preparation of 2-(2-((5-(1-aminoisoquinolin-7-yl)-2-cyclobutyl-2H-indazol-3-yl)methoxy)-4-methylphenyl)acetic acid (17e)

### Step-1: Preparation of methyl 5-bromo-2-cyclobutyl-2H-indazole-3-carboxylate (17a)

Compound 17a was prepared according to the procedure and method of purification reported in step-1 of scheme 8, from methyl 5-bromo-1H-indazole-3-carboxylate (1a) (3 g, 11.76 mmol) in THF, using DEAD (4.10 g, 23.52 mmol), PPh₃ (6.17 g, 23.52 mmol), and cyclobutanol (1.696 g, 23.52 mmol) to afford after workup and purification using method-N, methyl 5-bromo-2-cyclobutyl-2H-indazole-3-carboxylate (17a) (1.6 g, 5.18 mmol, 44.0% yield) as a white solid; 1H NMR (300 MHz, DMSO-d₆) δ 8.14 (d, J=1.9 Hz, 1H), 7.84 (d, J=9.0 Hz, 1H), 7.50 (dd, J=9.1, 1.9 Hz, 1H), 5.93 (p, J=8.2 Hz, 1H), 3.98 (s, 3H), 2.79-2.60 (m, 2H), 2.58-2.51 (m, 2H), 1.98-1.79 (m, 2H); MS (ES+): 309.00 & 311.00 (M+1).

### Step-2: Preparation of (5-bromo-2-cyclobutyl-2H-indazol-3-yl)methanol (17b)

Compound 17b was prepared according to the procedure and method of purification reported in step-2 of scheme 7, from methyl 5-bromo-2-cyclobutyl-2H-indazole-3-carboxylate (17a) (1.6 g, 5.18 mmol) in DCM (20 mL) using diisobutylaluminum hydride (1M solution in DCM, 12.94 mL, 12.94 mmol) to afford after workup and purification using method-O, (5-bromo-2-cyclobutyl-2H-indazol-3-yl)methanol (17b) (1.05 g, 72.2% yield) as a white solid; 1H NMR (300 MHz, DMSO-d₆) δ 8.01 (d, J=1.8 Hz, 1H), 7.59 (d, J=9.1 Hz, 1H), 7.30 (dd, J=9.1, 1.9 Hz, 1H), 5.44 (t, J=5.6 Hz, 1H), 5.28 (p, J=8.2 Hz, 1H), 4.85 (d, J=5.6 Hz, 2H), 2.78-2.60 (m, 2H), 2.47-2.37 (m, 2H), 1.95-1.80 (m, 2H); MS (ES+): 281.00 & 283.00 (M+1).

### Step-3: Preparation of ethyl 2-(2-((5-bromo-2-cyclobutyl-2H-indazol-3-yl)methoxy)-4-methylphenyl)acetate (17c)

Compound 17c was prepared according to the procedure and method of purification reported in step-2 of scheme 2, from (5-bromo-2-cyclobutyl-2H-indazol-3-yl)methanol (17b) (500 mg, 1.778 mmol) in DCM (10 mL), using ethyl 2-(2-hydroxy-4-methylphenyl)acetate (15a) (363 mg, 1.867 mmol), PPh₃ (560 mg, 2.134 mmol) and a solution of DCAD (784 mg, 2.134 mmol) in DCM (10 mL) to afford after workup and purification using method-N, ethyl 2-(2-((5-bromo-2-cyclobutyl-2H-indazol-3-yl)methoxy)-4-methylphenyl)acetate (17c) (200 mg, 24.59% yield) as a white oil; MS (ES+): 457.10 (M+1); MS (ES−): 454.90 (M−1).

### Step-4: Preparation of ethyl 2-(2-((5-(1-aminoisoquinolin-7-yl)-2-cyclobutyl-2H-indazol-3-yl)methoxy)-4-methylphenyl)acetate (17d)

Compound 17d was prepared according to the procedure and method of purification reported in step-5 of scheme 1, from ethyl 2-(2-((5-bromo-2-cyclobutyl-2H-indazol-3-yl)methoxy)-4-methylphenyl)acetate (17c) (100 mg, 0.219 mmol) in dioxane/THF (4 mL, 1:1) using 7-(4,4,5,5-tetramethyl-1,3,2-dioxaborolan-2-yl)isoquinolin-1-amine (9h) (70.9 mg, 0.262 mmol), K₃PO₄ (2M aqueous solution, 0.437 mL, 0.875 mmol), PCy₃ (12.26 mg, 0.044 mmol), Pd₂(dba)₃ (20.02 mg, 0.022 mmol) and PdCl₂(dppf)-CH₂Cl₂ adduct (17.7 mg, 0.022 mmol) to afford after workup and purification using method-Y, ethyl 2-(2-((5-(1-aminoisoquinolin-7-yl)-2-cyclobutyl-2H-indazol-3-yl)methoxy)-4-methylphenyl)acetate (17d) (80 mg, 70.3% yield) as a clear oil; MS (ES+): 521.30 (M+1).

### Step-5: Preparation of 2-(2-((5-(1-aminoisoquinolin-7-yl)-2-cyclobutyl-2H-indazol-3-yl)methoxy)-4-methylphenyl)acetic acid (17e)

Compound 17e was prepared according to the procedure and method of purification reported in step-2 of scheme 1, from ethyl 2-(2-((5-(1-aminoisoquinolin-7-yl)-2-cyclobutyl-2H-indazol-3-yl)methoxy)-4-methylphenyl)acetate (17d) (80 mg, 0.154 mmol) in MeOH/THF (6 mL, 1:1) using a solution of LiOH·H₂O (38.7 mg, 0.922 mmol) in water (2 mL) to afford after workup and purification using method-G, 2-(2-((5-(1-aminoisoquinolin-7-yl)-2-cyclobutyl-2H-indazol-3-yl)methoxy)-4-methylphenyl)acetic acid (17e) (20 mg, 26.4% yield) HCl salt as a white solid; 1H NMR (300 MHz, DMSO-d₆) δ 13.34 (s, 1H, D₂O exchangeable), 9.25 (s, 2H, D₂O exchangeable), 9.01 (s, 1H), 8.52-8.41 (m, 2H), 8.05 (d, J=8.5 Hz, 1H), 7.95-7.83 (m, 2H), 7.74-7.62 (m, 1H), 7.27 (d, J=6.9 Hz, 1H), 7.21 (s, 1H), 7.09 (d, J=7.6 Hz, 1H), 6.77 (d, J=7.6 Hz, 1H), 5.62 (s, 2H), 5.41-5.22 (m, 1H), 3.43 (s, 2H), 2.86-2.66 (m, 2H), 2.59-2.52 (m, 2H), 2.33 (s, 3H), 1.97-1.81 (m, 2H); MS (ES+): 493.20 (M+1); MS (ES−): 491.20 (M−1).

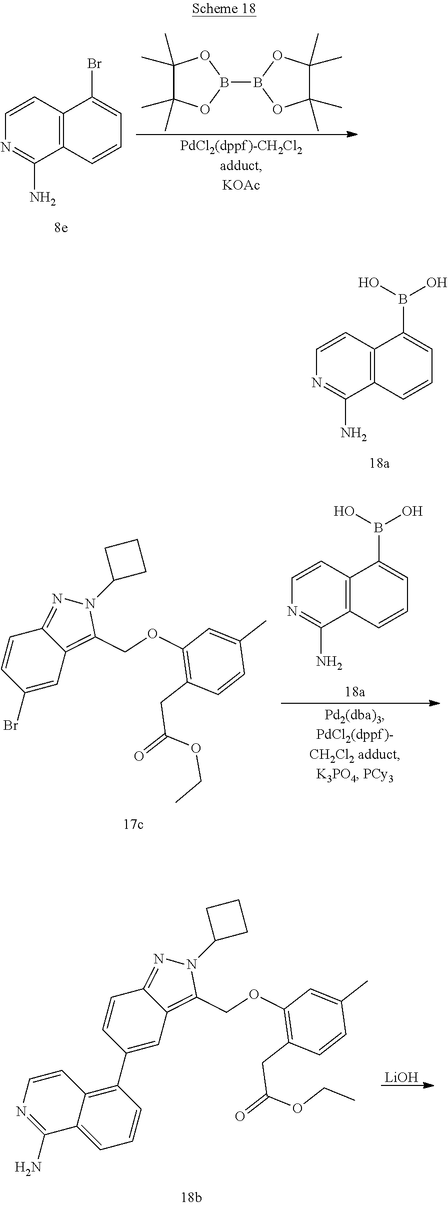

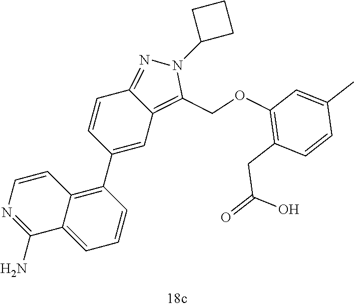 [SMILES: Cc1ccc(CC(=O)O)c(OCc2c3cc(-c4cccc5c(N)nccc45)ccc3nn2C2CCC2)c1]

### Preparation of 2-(2-((5-(1-aminoisoquinolin-5-yl)-2-cyclobutyl-2H-indazol-3-yl)methoxy)-4-methylphenyl)acetic acid (18c)

### Step-1: Preparation of (1-aminoisoquinolin-5-yl)boronic acid (18a)

Compound 18a was prepared according to the procedure and method of purification reported in step-4 of scheme 1, from 5-bromoisoquinolin-1-amine (8e) (5 g, 22.41 mmol; CAS #852570-80-0) in anhydrous dioxane (100 ml), using BISPIN (11.38 g, 44.8 mmol) and potassium acetate (5.50 g, 56.0 mmol), PdCl₂(dppf)-CH₂Cl₂ adduct (1.098 g, 1.345 mmol) and heating for 15 h at 100° C. This gave after workup and purification using method-Z, (1-aminoisoquinolin-5-yl)boronic acid (18a) (3.6 g, 85% yield), which was used as such for next step.

### Step-2: Preparation of ethyl 2-(2-((5-(1-aminoisoquinolin-5-yl)-2-cyclobutyl-2H-indazol-3-yl)methoxy)-4-methylphenyl)acetate (18b)

Compound 18b was prepared according to the procedure and method of purification reported in step-5 of scheme 1, from ethyl 2-(2-((5-bromo-2-cyclobutyl-2H-indazol-3-yl)methoxy)-4-methylphenyl)acetate (17c) (100 mg, 0.219 mmol) in dioxane/THF (4 mL, 1:1), using (1-aminoisoquinolin-5-yl)boronic acid (18a) (61.7 mg, 0.328 mmol), K₃PO₄ (2M aqueous solution, 0.437 mL, 0.875 mmol), PCy₃ (12.26 mg, 0.044 mmol), Pd₂(dba)₃ (20.02 mg, 0.022 mmol) and PdCl₂(dppf)-CH₂Cl₂ adduct (17.86 mg, 0.022 mmol) to afford after workup and purification using method-Y, ethyl 2-(2-((5-(1-aminoisoquinolin-5-yl)-2-cyclobutyl-2H-indazol-3-yl)methoxy)-4-methylphenyl)acetate (18b) (80 mg, 70% yield) as a clear oil; MS (ES+): 521.30 (M+1).

### Step-3: Preparation of 2-(2-((5-(1-aminoisoquinolin-5-yl)-2-cyclobutyl-2H-indazol-3-yl)methoxy)-4-methylphenyl)acetic acid (18c)

Compound 18c was prepared according to the procedure and method of purification reported in step-2 of scheme 1, from ethyl 2-(2-((5-(1-aminoisoquinolin-5-yl)-2-cyclobutyl-2H-indazol-3-yl)methoxy)-4-methylphenyl)acetate (18b) (80 mg, 0.154 mmol) in MeOH/THF (6 mL, 1:1) using a solution of LiOH·H₂O (38.7 mg, 0.922 mmol) in water (2 mL) to afford after workup and purification using method-G, 2-(2-((5-(1-aminoisoquinolin-5-yl)-2-cyclobutyl-2H-indazol-3-yl)methoxy)-4-methylphenyl)acetic acid (18c) (8 mg, 10.57% yield) HCl salt as a white solid; 1H NMR (300 MHz, DMSO-d₆) δ 13.18 (s, 1H, D₂O exchangeable), 9.16 (s, 2H, D₂O exchangeable), 8.61 (d, J=8.3 Hz, 1H), 7.96 (d, J=8.0 Hz, 2H), 7.85 (m, 2H), 7.63 (d, J=7.3 Hz, 1H), 7.32 (dd, J=8.8, 1.6 Hz, 1H), 7.11 (s, 1H), 7.07 (d, J=7.5 Hz, 1H), 6.98 (d, J=7.2 Hz, 1H), 6.75 (d, J=7.6 Hz, 1H), 5.57 (s, 2H), 5.42-5.26 (m, 1H), 3.42 (s, 2H), 2.83-2.71 (m, 2H), 2.57-2.53 (m, 2H), 2.28 (s, 3H), 1.99-1.86 (m, 2H); MS (ES+): 493.20 (M+1); MS (ES−): 491.20 (M−1).

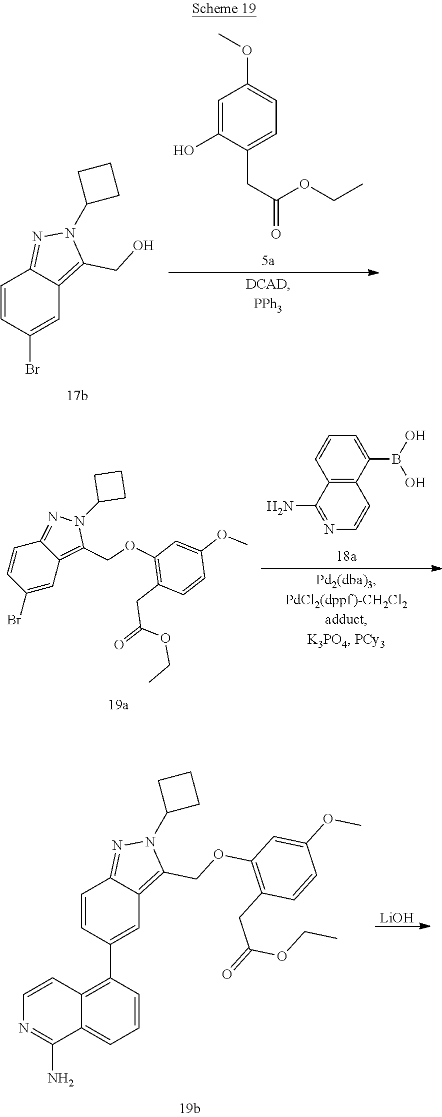 [SMILES: OCc1c2cc(Br)ccc2nn1C1CCC1 ; CCOC(=O)Cc1ccc(OC)cc1O ; CCOC(=O)Cc1ccc(OC)cc1OCc1c2cc(Br)ccc2nn1C1CCC1 ; Nc1nccc2c(B(O)O)cccc12 ; CCOC(=O)Cc1ccc(OC)cc1OCc1c2cc(-c3cccc4c(N)nccc34)ccc2nn1C1CCC1 ; "LiOH"]

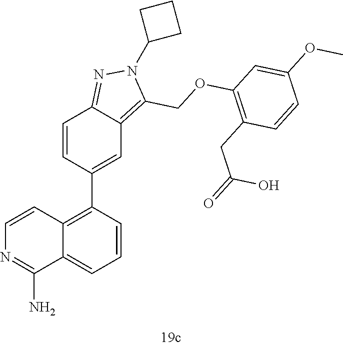 [SMILES: COc1ccc(CC(=O)O)c(OCc2c3cc(-c4cccc5c(N)nccc45)ccc3nn2C2CCC2)c1]

### Preparation of 2-(2-((5-(1-aminoisoquinolin-5-yl)-2-cyclobutyl-2H-indazol-3-yl)methoxy)-4-methoxyphenyl)acetic acid (19c)

### Step-1: Preparation of ethyl 2-(2-((5-bromo-2-cyclobutyl-2H-indazol-3-yl)methoxy)-4-methoxyphenyl)acetate (19a)

Compound 19a was prepared according to the procedure and method of purification reported in step-2 of scheme 2, from (5-bromo-2-cyclobutyl-2H-indazol-3-yl)methanol (17b) (500 mg, 1.778 mmol) in DCM (10 mL), using ethyl 2-(2-hydroxy-4-methoxyphenyl)acetate (5a) (393 mg, 1.867 mmol), PPh₃ (560 mg, 2.134 mmol) and a solution of DCAD (784 mg, 2.134 mmol) in DCM (10 mL) to afford after workup and purification using method-N, ethyl 2-(2-((5-bromo-2-cyclobutyl-2H-indazol-3-yl)methoxy)-4-methoxyphenyl)acetate (19a) (250 mg, 29.7% yield) as a white oil; MS (ES+): 473.10 & 475.10 (M+1); MS (ES−): 471.00 & 473.10 (M−1).

### Step-2: Preparation of ethyl 2-(2-((5-(1-aminoisoquinolin-5-yl)-2-cyclobutyl-2H-indazol-3-yl)methoxy)-4-methoxyphenyl)acetate (19b)

Compound 19b was prepared according to the procedure and method of purification reported in step-5 of scheme 1, from ethyl 2-(2-((5-bromo-2-cyclobutyl-2H-indazol-3-yl)methoxy)-4-methoxyphenyl)acetate (19a) (100 mg, 0.211 mmol) in dioxane/THF (4 mL, Ratio: 1:1), using (1-aminoisoquinolin-5-yl)boronic acid (18a) (59.6 mg, 0.317 mmol). K₃PO₄ (2M aqueous solution, 0.423 mL, 0.845 mmol), PCy₃ (11.85 mg, 0.042 mmol), Pd₂(dba)₃ (19.35 mg, 0.021 mmol) and PdCl₂(dppf)-CH₂Cl₂ adduct (17.3 mg, 0.021 mmol) to afford after workup and purification using method-Y, ethyl 2-(2-((5-(1-aminoisoquinolin-5-yl)-2-cyclobutyl-2H-indazol-3-yl)methoxy)-4-methoxyphenyl)acetate (19b) (60 mg, 0.112 mmol, 52.9% yield) as a clear oil.

MS (ES+): 537.25 (M+1).

### Step-3: Preparation of 2-(2-((5-(1-aminoisoquinolin-5-yl)-2-cyclobutyl-2H-indazol-3-yl)methoxy)-4-methoxyphenyl)acetic acid (19c)

Compound 19c was prepared according to the procedure and method of purification reported in step-2 of scheme 1, from ethyl 2-(2-((5-(1-aminoisoquinolin-5-yl)-2-cyclobutyl-2H-indazol-3-yl)methoxy)-4-methoxyphenyl)acetate (19b) (60 mg, 0.112 mmol) in MeOH/THF (6 mL, 1:1) using a solution of LiOH·H₂O (28.2 mg, 0.671 mmol) in water (2 mL) to afford after workup and purification using method-G, 2-(2-((5-(1-aminoisoquinolin-5-yl)-2-cyclobutyl-2H-indazol-3-yl)methoxy)-4-methoxyphenyl)acetic acid (19c) (20 mg, 35.2% yield) HCl salt as a white solid; 1H NMR (300 MHz, DMSO-d₆) δ 13.38 (s, 1H, D₂O exchangeable), 9.25 (s, 2H, DO exchangeable), 8.64 (d, J=8.3 Hz, 1H), 8.03-7.90 (m, 2H), 7.90-7.78 (m, 2H), 7.64 (d, J=7.3 Hz, 1H), 7.32 (dd, J=8.9, 1.6 Hz, 1H), 7.09 (d, J=8.3 Hz, 1H), 6.97 (d, J=7.2 Hz, 1H), 6.85 (d, J=2.3 Hz, 1H), 6.51 (dd, J=8.3, 2.3 Hz, 1H), 5.60 (s, 2H), 5.42-5.25 (m, 1H), 3.71 (s, 3H), 3.39 (s, 2H), 2.86-2.65 (m, 2H), 2.54-2.51 (m, 2H), 2.01-1.83 (m, 2H); MS (ES+): 509.20 (M+1); MS (ES−): 507.20 (M−1).

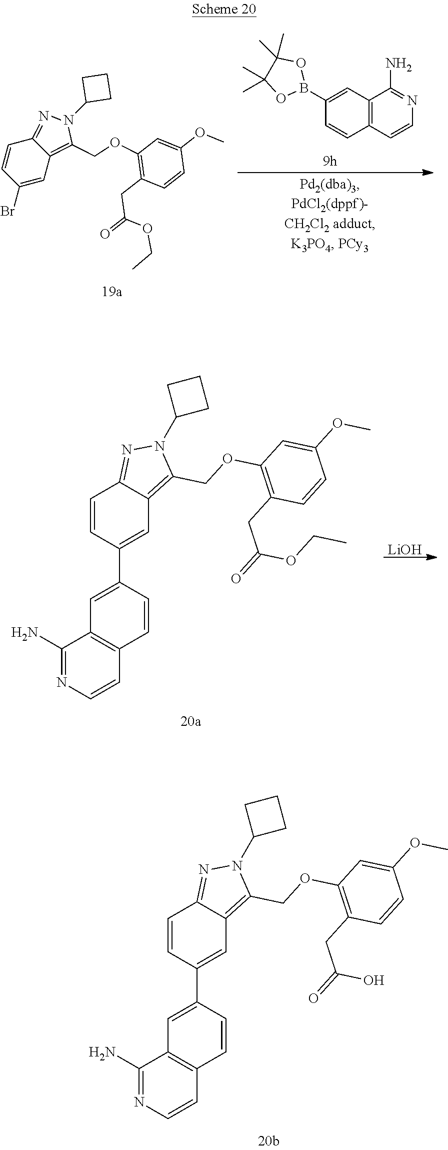

### Preparation of 2-(2-((5-(1-aminoisoquinolin-7-yl)-2-cyclobutyl-2H-indazol-3-yl)methoxy)-4-methoxyphenyl)acetic acid (20b)

### Step-1: Preparation of ethyl 2-(2-((5-(1-aminoisoquinolin-7-yl)-2-cyclobutyl-2H-indazol-3-yl)methoxy)-4-methoxyphenyl)acetate (20a)

Compound 20a was prepared according to the procedure and method of purification reported in step-5 of scheme 1, from ethyl 2-(2-((5-bromo-2-cyclobutyl-2H-indazol-3-yl)methoxy)-4-methoxyphenyl)acetate (19a) (100 mg, 0.211 mmol) in dioxane/THF (4 mL, 1:1) using 7-(4,4,5,5-tetramethyl-1,3,2-dioxaborolan-2-yl)isoquinolin-1-amine (9h) (68.5 mg, 0.254 mmol), K₃PO₄ (2M aqueous solution, 0.423 mL, 0.845 mmol), PCy₃ (11.85 mg, 0.042 mmol), Pd₂(dba)₃ (19.35 mg, 0.021 mmol) and PdCl₂(dppf)-CH₂Cl₂ adduct to afford after workup and purification using method-Y, ethyl 2-(2-((5-(1-aminoisoquinolin-7-yl)-2-cyclobutyl-2H-indazol-3-yl)methoxy)-4-methoxyphenyl)acetate (20a) (60 mg, 52.9% yield) as a clear oil; MS (ES+): 537.30 (M+1).

### Step-2: Preparation of 2-(2-((5-(1-aminoisoquinolin-7-yl)-2-cyclobutyl-2H-indazol-3-yl)methoxy)-4-methoxyphenyl)acetic acid (20b)

Compound 20b was prepared according to the procedure and method of purification reported in step-2 of scheme 1, from ethyl 2-(2-((5-(1-aminoisoquinolin-7-yl)-2-cyclobutyl-2H-indazol-3-yl)methoxy)-4-methoxyphenyl)acetate (20a) (60 mg, 0.112 mmol) in MeOH/THF (6 mL, 1:1) using a solution of LiOH·H₂O (28.2 mg, 0.671 mmol) in water (2 mL) to afford after workup and purification using method-G, 2-(2-((5-(1-aminoisoquinolin-7-yl)-2-cyclobutyl-2H-indazol-3-yl)methoxy)-4-methoxyphenyl)acetic acid (20b) (20 mg, 35.2% yield) HCl salt as a white solid; ¹H NMR (300 MHz, DMSO-d₆) δ 13.18 (s, 1H, D₂O) exchangeable), 9.17 (s, 2H, D₂O exchangeable), 8.98 (s, 1H), 8.54-8.39 (m, 2H), 8.06 (d, J=8.5 Hz, 1H), 7.95-7.82 (m, 2H), 7.68 (d, J=7.2 Hz, 1H), 7.27 (d, J=6.9 Hz, 1H), 7.11 (d, J=8.3 Hz, 1H), 6.93 (d, J=2.4 Hz, 1H), 6.53 (dd, J=8.3, 2.3 Hz, 1H), 5.65 (s, 2H), 5.39-5.24 (m, 1H), 3.76 (s, 3H), 3.41 (s, 2H), 2.82-2.70 (m, 2H), 2.57-2.53 (m, 2H), 1.98-1.83 (m, 2H); MS (ES+): 509.20 (M+1); MS (ES−): 507.20 (M−1).

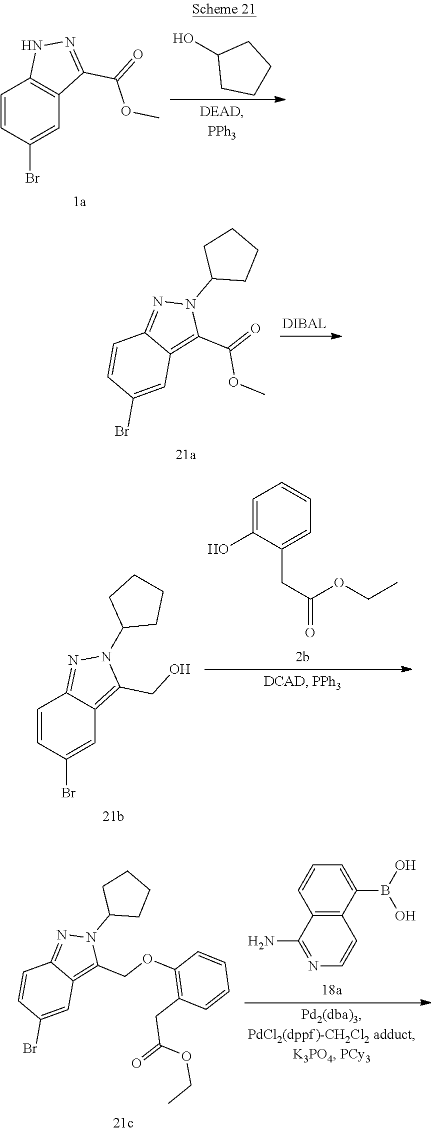

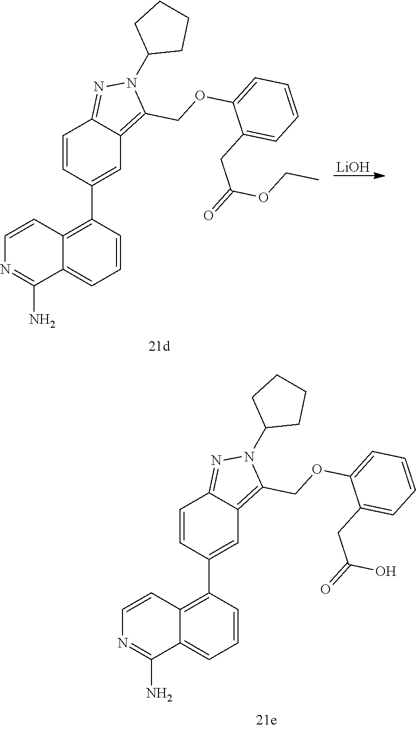

### Preparation of 2-(2-((5-(1-aminoisoquinolin-5-yl)-2-cyclopentyl-2H-indazol-3-yl)methoxy)phenyl)acetic acid (21e)

### Step-1: Preparation of methyl 5-bromo-2-cyclopentyl-2H-indazole-3-carboxylate (21a)

Compound 21a was prepared according to the procedure and method of purification reported in step-1 of scheme 8, from methyl 5-bromo-1H-indazole-3-carboxylate (1a) (3 g, 11.76 mmol) in THF, using DEAD (4.10 g, 23.52 mmol), PPh₃ (6.17 g, 23.52 mmol) and cyclopentanol (2.026 g, 23.52 mmol) to afford after workup and purification using method-N, methyl 5-bromo-2-cyclopentyl-2H-indazole-3-carboxylate (21a) (2.7 g, 71.0% yield) as a white solid; 1H NMR (300 MHz, DMSO-d₆) δ 8.14 (dd, J=2.0, 0.7 Hz, 1H), 7.79 (dd, J=9.1, 0.7 Hz, 1H), 7.47 (dd, J=9.0, 1.9 Hz, 1H), 5.96 (tt, J=7.8, 6.0 Hz, 1H), 3.98 (s, 3H), 2.27-2.01 (m, 4H), 1.99-1.82 (m, 2H), 1.79-1.61 (m, 2H); MS (ES+): 323.00 & 325.00 (M+1).

### Step-2: Preparation of (5-bromo-2-cyclopentyl-2H-indazol-3-yl)methanol (21b)

Compound 21b was prepared according to the procedure and method of purification reported in step-2 of scheme 7, from methyl 5-bromo-2-cyclopentyl-2H-indazole-3-carboxylate (21a) (2.7 g, 8.35 mmol) in DCM (100 mL) using diisobutylaluminum hydride (1M solution in DCM, 20.89 mL, 20.89 mmol) to afford after workup and purification using method-W, (5-bromo-2-cyclopentyl-2H-indazol-3-yl)methanol (21b) (2.1 g, 7.11 mmol, 85% yield) as a light yellow oil; MS (ES+): 295.00 & 297.00 (M+1).

### Step-3: Preparation of ethyl 2-(2-((5-bromo-2-cyclopentyl-2H-indazol-3-yl)methoxy)phenyl)acetate (21c)

Compound 21c was prepared according to the procedure and method of purification reported in step-2 of scheme 2 from, (5-bromo-2-cyclopentyl-2H-indazol-3-yl)methanol (21b) (1 g, 3.39 mmol), using ethyl 2-(2-hydroxyphenyl)acetate (2b) (0.672 g, 3.73 mmol), PPh₃ (1.066 g, 4.07 mmol) and a solution of DCAD (1.493 g, 4.07 mmol) in DCM (10 mL) to afford after workup and purification using method-N, ethyl 2-(2-((5-bromo-2-cyclopentyl-2H-indazol-3-yl)methoxy)phenyl)acetate (21c) (560 mg, 36.1% yield) as a white oil; ¹H NMR (300 MHZ, DMSO-d₆) δ 8.11 (d, J=1.8 Hz, 1H), 7.62 (d, J=9.1 Hz, 1H), 7.38-7.26 (m, 3H), 7.22 (dd. J=7.4, 1.5 Hz, 1H), 7.01-6.91 (m, 1H), 5.57 (s, 2H), 5.17 (p, J=7.2 Hz, 1H), 3.80 (q, J=7.1 Hz, 2H), 3.51 (s, 2H), 2.21-2.06 (m, 4H), 1.97-1.85 (m, 2H), 1.74-1.58 (m, 2H), 0.88 (t, J=7.1 Hz, 3H); MS (ES+): 457.10 & 459.05 (M+1); MS (ES−): 455.00 & 457.10 (M−1).

### Step-4: Preparation of ethyl 2-(2-((5-(1-aminoisoquinolin-5-yl)-2-cyclopentyl-2H-indazol-3-yl)methoxy)phenyl)acetate (21d)

Compound 21d was prepared according to the procedure and method of purification reported in step-5 of scheme 1, from ethyl 2-(2-((5-bromo-2-cyclopentyl-2H-indazol-3-yl)methoxy)phenyl)acetate (21c) (200 mg, 0.437 mmol) in dioxane/THF (4 mL), using (1-aminoisoquinolin-5-yl)boronic acid (18a) (123 mg, 0.656 mmol), K₃PO₄ (2M aqueous solution, 0.875 mL, 1.749 mmol), PCy₃ (24.53 mg, 0.087 mmol). Pd₂(dba)₃ (40.0 mg, 0.044 mmol) and PdCl₂(dppf)-CH₂Cl₂ adduct (35.7 mg, 0.044 mmol) to afford after workup and purification using method-Y, ethyl 2-(2-((5-(1-aminoisoquinolin-5-yl)-2-cyclopentyl-2H-indazol-3-yl)methoxy)phenyl)acetate (21d) (150 mg, 65.9% yield) as a clear oil; MS (ES+): 521.20 (M+1).

### Step-5: Preparation of 2-(2-((5-(1-aminoisoquinolin-5-yl)-2-cyclopentyl-2H-indazol-3-yl)methoxy)phenyl)acetic acid (21e)

Compound 21e was prepared according to the procedure and method of purification reported in step-2 of scheme 1, from ethyl 2-(2-((5-(1-aminoisoquinolin-5-yl)-2-cyclopentyl-2H-indazol-3-yl)methoxy)phenyl)acetate (21d) (150 mg, 0.288 mmol) in MeOH/THF (6 mL, 1:1) using a solution of LiOH·H₂O (72.5 mg, 1.729 mmol) in water (2 mL) to afford after workup and purification using method-G, 2-(2-((5-(1-aminoisoquinolin-5-yl)-2-cyclopentyl-2H-indazol-3-yl)methoxy)phenyl)acetic acid (21e) (35 mg, 24.66% yield) HCl salt as a white solid; 1H NMR (300 MHz, DMSO-d₆) δ 13.19 (s, 1H, D₂O exchangeable), 12.15 (s, 1H, D₂O) exchangeable), 9.16 (s, 2H, D₂O exchangeable), 8.61 (d, J=8.3 Hz, 1H), 7.96 (d, J=5.9 Hz, 2H), 7.86 (t, J=7.8 Hz, 1H), 7.80 (d, J=8.8 Hz, 1H), 7.63 (d, J=7.3 Hz, 1H), 7.35-7.24 (m, 3H), 7.21 (d, J=7.4 Hz, 1H), 7.03-6.90 (m, 2H), 5.63 (s, 2H), 5.36-5.07 (m, 1H), 3.49 (s, 2H), 2.31-2.17 (m, 2H), 2.17-2.06 (m, 2H), 2.04-1.90 (m, 2H), 1.80-1.62 (m, 2H); MS (ES+): 493.20 (M+1); MS (ES−): 491.20 (M−1).

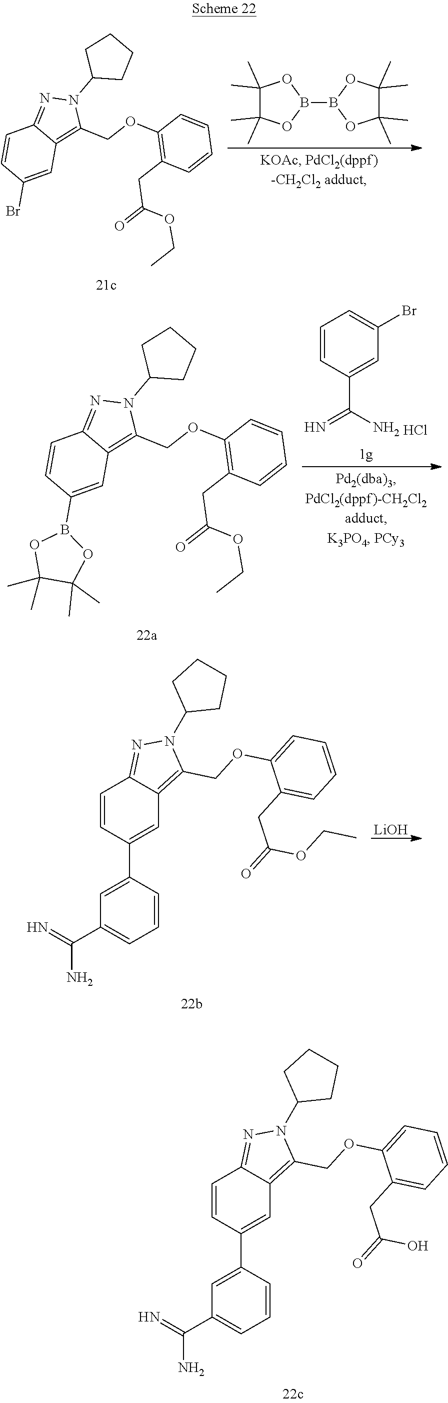

### Preparation of 2-(2-((5-(3-carbamimidoylphenyl)-2-cyclopentyl-2H-indazol-3-yl)methoxy)phenyl)acetic acid (22c)

### Step-1: Preparation of ethyl 2-(2-((2-cyclopentyl-5-(4,4,5,5-tetramethyl-1,3,2-dioxaborolan-2-yl)-2H-indazol-3-yl)methoxy)phenyl)acetate (22a)

Compound 22a was prepared according to the procedure and method of purification reported in step-4 of scheme 1, from ethyl 2-(2-((5-bromo-2-cyclopentyl-2H-indazol-3-yl)methoxy)phenyl)acetate (21c) (450 mg, 0.984 mmol) in anhydrous dioxane (20 mL), using BISPIN (500 mg, 1.968 mmol), KOAc (241 mg, 2.460 mmol), PdCl₂(dppf)-CH₂Cl₂ adduct (48.2 mg, 0.059 mmol) and heating for 3 h at 100° ° C. This gave after workup and purification using method-AA, ethyl 2-(2-((2-cyclopentyl-5-(4,4,5,5-tetramethyl-1,3,2-dioxaborolan-2-yl)-2H-indazol-3-yl)methoxy)phenyl)acetate (22a) (180 mg, 36.3% yield) as a clear oil; MS (ES+): 505.30 (M+1); MS (ES−): 503.05 (M−1).

### Step-2: Preparation of ethyl 2-(2-((5-(3-carbamimidoylphenyl)-2-cyclopentyl-2H-indazol-3-yl)methoxy)phenyl)acetate (22b)

Compound 22b was prepared according to the procedure and method of purification reported in step-5 of scheme 1, from ethyl 2-(2-((2-cyclopentyl-5-(4,4,5,5-tetramethyl-1,3,2-dioxaborolan-2-yl)-2H-indazol-3-yl)methoxy)phenyl)acetate (22a) (180 mg, 0.357 mmol) in dioxane/THF (4 mL) using 3-bromobenzimidamide hydrochloride (1g) (168 mg, 0.714 mmol), K₃PO₄ (2M aqueous solution, 0.714 mL, 1.427 mmol), PCy₃ (20.01 mg, 0.071 mmol), Pd₂(dba)₃ (32.7 mg, 0.036 mmol) and PdCl₂(dppf)-CH₂Cl₂ adduct (39.1 mg, 0.036 mmol) to afford after workup and purification using method-F, ethyl 2-(2-((5-(3-carbamimidoylphenyl)-2-cyclopentyl-2H-indazol-3-yl)methoxy)phenyl)acetate (22b) (120 mg, 67.7% yield) as a clear oil; ¹H NMR (300 MHz, DMSO-d₆) δ 8.70 (s, 3H), 8.24 (s, 1H), 8.11 (s, 1H), 8.01 (d, J=7.8 Hz, 1H), 7.82-7.69 (m, 4H), 7.64 (t, J=7.7 Hz, 1H), 7.34 (d, J=4.2 Hz, 2H), 7.23 (d, J=7.4 Hz, 1H), 7.02-6.90 (m, 1H), 5.65 (s, 2H), 5.27-5.11 (m, 1H), 3.76 (q, J=7.1 Hz, 2H), 3.53 (s, 2H), 2.22-2.10 (m, 4H), 2.00-1.92 (m, 2H), 1.73-1.66 (m, 2H), 0.86 (t, J=7.1 Hz, 3H); MS (ES+): 497.20 (M+1); MS (ES−): 495.10 (M−1).

### Step-3: Preparation of 2-(2-((5-(3-carbamimidoylphenyl)-2-cyclopentyl-2H-indazol-3-yl)methoxy)phenyl)acetic acid (22c)

Compound 22c was prepared according to the procedure and method of purification reported in step-2 of scheme 1, from ethyl 2-(2-((5-(3-carbamimidoylphenyl)-2-cyclopentyl-2H-indazol-3-yl)methoxy)phenyl)acetate (22b) (120 mg, 0.242 mmol) in MeOH/THF (6 mL, 1:1) using a solution of LiOH·H₂O (60.8 mg, 1.450 mmol) in water (2 mL) to afford after workup and purification using method-G, 2-(2-((5-(3-carbamimidoylphenyl)-2-cyclopentyl-2H-indazol-3-yl)methoxy)phenyl)acetic acid (22c) (30 mg, 26.5% yield) HCl salt as a white solid; ¹H NMR (300 MHz, DMSO-d₆) δ 9.47 (s, 2H, D₂O exchangeable), 9.15 (s, 2H, D₂O exchangeable), 8.34 (s, 1H), 8.17 (s, 1H), 8.11 (d, J=7.6 Hz, 1H), 7.83-7.75 (m, 3H), 7.74-7.64 (m, 1H), 7.39-7.26 (m, 2H), 7.22 (d, J=7.3 Hz, 1H), 6.96 (t, J=7.2 Hz, 1H), 5.65 (s, 2H), 5.31-5.08 (m, 1H), 3.46 (s, 2H), 2.31-2.15 (m, 2H), 2.15-2.03 (m, 2H), 2.01-1.87 (m, 2H), 1.78-1.63 (m, 2H); MS (ES+): 469.20 (M+1); MS (ES−): 467.10 (M−1).

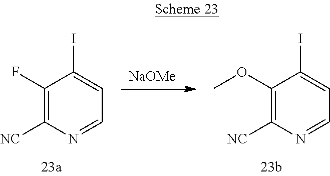

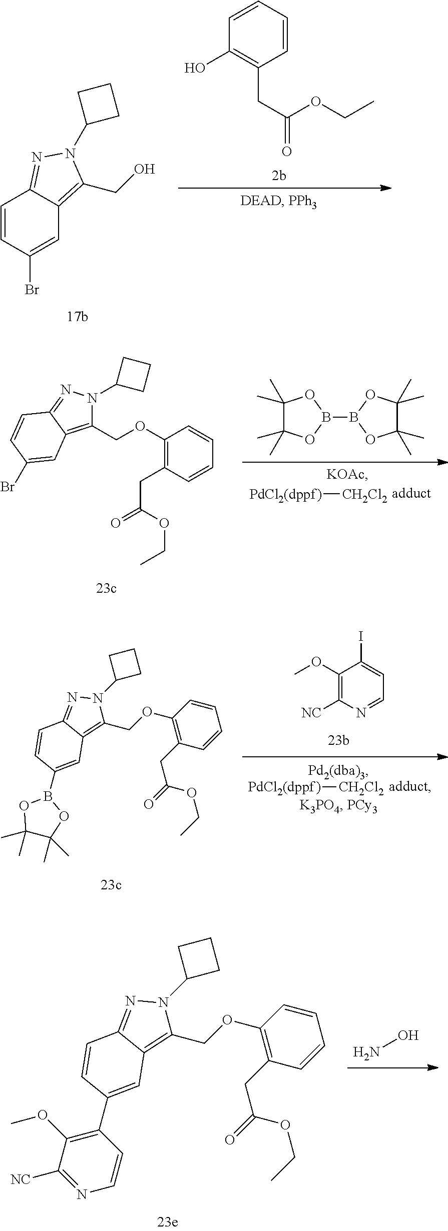

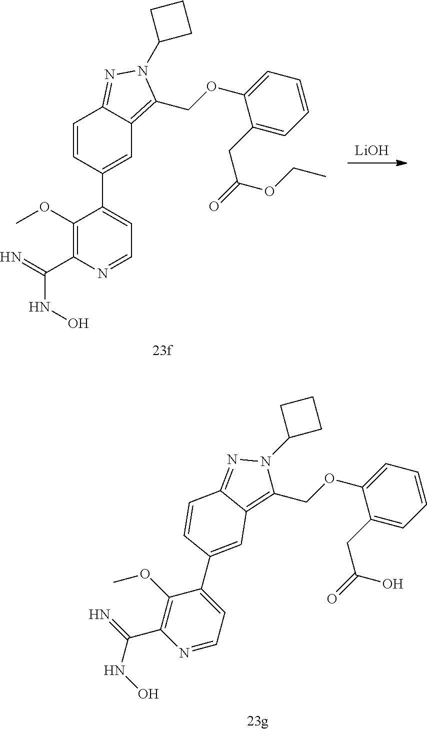

### Preparation of 2-(2-((2-cyclobutyl-5-(2-(N-hydroxycarbamimidoyl)-3-methoxypyridin-4-yl)-2H-indazol-3-yl)methoxy)phenyl)acetic acid (23g)

### Step-1: Preparation of 4-iodo-3-methoxypicolinonitrile (23b)

A solution of 3-fluoro-4-iodopicolinonitrile (23a) (1 g, 4.03 mmol; CAS #669066-35-7) in sodium methoxide (25 wt % solution in methanol) (4.49 mL, 20.16 mmol) was stirred for 2 hours at 0° C. and 2 hours at ambient temperature. The reaction was diluted with ethyl acetate washed with water, brine dried and concentrated in vacuum. The obtained residue was purified using method-AB, to give 4-iodo-3-methoxypicolinonitrile (23b) (380 mg, 36.2% yield) as a white solid; 1H NMR (300 MHz, DMSO-d₆) δ 8.28 (d, J=4.9 Hz, 1H), 8.13 (d, J=4.9 Hz, 1H), 4.03 (s, 3H); MS (ES+): 260.90 (M+1).

### Step-2: Preparation of ethyl 2-(2-((5-bromo-2-cyclobutyl-2H-indazol-3-yl)methoxy)phenyl)acetate (23c)

Compound 23c was prepared according to the procedure and method of purification reported in step-1 of scheme 8, from (5-bromo-2-cyclobutyl-2H-indazol-3-yl)methanol (17b) (820 mg, 2.92 mmol) in THF (30 mL), using PPh₃ (1530 mg, 5.83 mmol), ethyl 2-(2-hydroxyphenyl)acetate (2b) (788 mg, 4.37 mmol) and DEAD (1016 mg, 5.83 mmol) to afford after workup and purification using method-N, ethyl 2-(2-((5-bromo-2-cyclobutyl-2H-indazol-3-yl)methoxy)phenyl)acetate (23c) (430 mg, 33.3% yield) as a yellow solid; 1H NMR (300 MHz, DMSO-d₆) δ 7.94 (d, J=1.8 Hz, 1H), 7.70 (d, J=8.9 Hz, 1H), 7.52 (dd, J=9.0, 1.8 Hz, 1H), 7.34-7.14 (m, 3H), 6.92 (td, J=7.1, 1.7 Hz, 1H), 5.39 (s, 2H), 5.29 (p. J=8.3 Hz, 1H), 3.91 (q, J=7.1 Hz, 2H), 3.53 (s, 2H), 2.70-2.53 (m, 2H), 2.48-2.38 (m, 2H), 1.95-1.77 (m, 2H), 0.93 (t, J=7.1 Hz, 3H); MS (ES+): 443.10 & 445.10 (M+1); MS (ES+): 465.05 & 467.10 (M+Na).

### Step-3: Preparation of ethyl 2-(2-((2-cyclobutyl-5-(4,4,5,5-tetramethyl-1,3,2-dioxaborolan-2-yl)-2H-indazol-3-yl)methoxy)phenyl)acetate (23d)

Compound 23d was prepared according to the procedure and method of purification reported in step-4 of scheme 1, from ethyl 2-(2-((5-bromo-2-cyclobutyl-2H-indazol-3-yl)methoxy)phenyl)acetate (23c) (430 mg, 0.970 mmol) in anhydrous dioxane (15 mL), using BISPIN (493 mg, 1.940 mmol), KOAc (238 mg, 2.425 mmol) and PdCl₂(dppf)-CH₂Cl₂ adduct (47.5 mg, 0.058 mmol) to afford after workup and purification using method-B, ethyl 2-(2-((2-cyclobutyl-5-(4,4,5,5-tetramethyl-1,3,2-dioxaborolan-2-yl)-2H-indazol-3-yl)methoxy)phenyl)acetate (23d) (280 mg, 58.9% yield) as a clear oil; ¹H NMR (300 MHz. DMSO-d₆) δ 8.22 (s, 1H), 7.63 (d, J=8.7 Hz, 1H), 7.48 (d, J=8.7 Hz, 1H), 7.32 (d, J=4.6 Hz, 2H), 7.21 (d, J=7.4 Hz, 1H), 7.01-6.89 (m, 1H), 5.57 (s, 2H), 5.30 (p, J=8.2 Hz, 1H), 3.77 (q. J=7.1 Hz, 2H), 3.49 (s, 2H), 2.85-2.66 (m, 2H), 2.48-2.38 (m, 2H), 1.97-1.79 (m, 2H), 1.29 (s, 12H), 0.86 (t, J=7.1 Hz, 3H); MS (ES+): 491.30 (M+1).

### Step-4: Preparation of ethyl 2-(2-((5-(2-cyano-3-methoxypyridin-4-yl)-2-cyclobutyl-2H-indazol-3-yl)methoxy)phenyl)acetate (23e)

Compound 23e was prepared according to the procedure and method of purification reported in step-5 of scheme 1, from ethyl 2-(2-((2-cyclobutyl-5-(4,4,5,5-tetramethyl-1,3,2-dioxaborolan-2-yl)-2H-indazol-3-yl)methoxy)phenyl)acetate (23d) (280 mg, 0.571 mmol) in dioxane/THF (4 mL, 1:1), using 4-iodo-3-methoxypicolinonitrile (23b) (178 mg, 0.685 mmol). K₃PO₄ (2M aqueous solution, 1.142 mL, 2.284 mmol). PCy₃ (32.0 mg, 0.114 mmol). Pd₂(dba)₃ (52.3 mg, 0.057 mmol) and PdCl₂(dppf)-CH₂CL adduct (46.6 mg, 0.057 mmol) to afford after workup and purification using method-AC, ethyl 2-(2-((5-(2-cyano-3-methoxypyridin-4-yl)-2-cyclobutyl-2H-indazol-3-yl)methoxy)phenyl)acetate (23e) (280 mg, 99% yield) as a clear oil; 1H NMR (300 MHz, DMSO-d₆) δ 8.55 (d, J=4.9 Hz, 1H), 8.18 (s, 1H), 7.87-7.76 (m, 2H), 7.55 (d, J=9.1 Hz, 1H), 7.32 (d, J=6.6 Hz, 2H), 7.22 (d, J=7.4 Hz, 1H), 6.96 (t, J=7.0 Hz, 1H), 5.60 (s, 2H), 5.34 (p, J=8.3 Hz, 1H), 3.76 (q, J=7.1 Hz. 2H), 3.63 (s, 3H), 3.52 (s, 2H), 2.88-2.69 (m, 2H), 2.50-2.45 (m, 2H), 1.96-1.84 (m, 2H), 0.87 (t, J=7.1 Hz, 3H); MS (ES+): 497.20 (M+1).

### Step-5: Preparation of ethyl 2-(2-((2-cyclobutyl-5-(2-(N-hydroxycarbamimidoyl)-3-methoxypyridin-4-yl)-2H-indazol-3-yl)methoxy)phenyl)acetate (23f)

To a solution of ethyl 2-(2-((5-(2-cyano-3-methoxypyridin-4-yl)-2-cyclobutyl-2H-indazol-3-yl)methoxy)phenyl)acetate (23e) (280 mg, 0.564 mmol) in EtOH (10 mL) was added hydroxylamine (0.373 mL, 5.64 mmol) and heated at reflux for 3 h. Solvent was removed and the residue obtained was purified using method-E, to afford ethyl 2-(2-((2-cyclobutyl-5-(2-(N-hydroxycarbamimidoyl)-3-methoxypyridin-4-yl)-2H-indazol-3-yl)methoxy)phenyl)acetate (23f) (280 mg, 0.529 mmol, 94% yield) as a white solid; 1H NMR (300 MHz, DMSO-d₆) δ 9.68 (s, 1H), 8.39 (d, J=4.9 Hz, 1H), 8.08 (s, 1H), 7.78 (d, J=8.9 Hz, 1H), 7.53 (d, J=9.0 Hz, 1H), 7.47 (d, J=4.9 Hz, 1H), 7.31 (s, 2H), 7.22 (d, J=7.5 Hz, 1H), 6.96 (s, 1H), 5.60 (s, 2H), 5.41-5.25 (m, 1H), 3.77 (q, J=7.1 Hz, 2H), 3.53 (s, 2H), 3.45 (s, 3H), 2.85-2.69 (m, 2H), 2.51-2.46 (m, 2H), 1.98-1.83 (m, 2H), 0.88 (t, J=7.1 Hz, 3H); MS (ES+): 530.20 (M+1).

### Step-6: Preparation of 2-(2-((2-cyclobutyl-5-(2-(N-hydroxycarbamimidoyl)-3-methoxypyridin-4-yl)-2H-indazol-3-yl)methoxy)phenyl)acetic acid (23g)

Compound 23g was prepared according to the procedure and method of purification reported in step-2 of scheme 1, from ethyl 2-(2-((2-cyclobutyl-5-(2-(N-hydroxycarbamimidoyl)-3-methoxypyridin-4-yl)-2H-indazol-3-yl)methoxy)phenyl)acetate (23f) (100 mg, 0.189 mmol) in MeOH/THF (6 mL, 1:1) using a solution of LiOH·H₂O (47.5 mg, 1.133 mmol) in water (2 mL) to afford after workup and purification using method-G, 2-(2-((2-cyclobutyl-5-(2-(N-hydroxycarbamimidoyl)-3-methoxypyridin-4-yl)-2H-indazol-3-yl)methoxy)phenyl)acetic acid (23g) (15 mg, 15.84% yield) HCl salt as a white solid; ¹H NMR (300 MHz, DMSO-d₆) δ 9.32 (s, 2H, D₂O exchangeable), 8.54 (d, J=4.9 Hz, 1H), 8.22 (s, 1H), 7.88-7.71 (m, 2H), 7.57 (d, J=9.0 Hz, 1H), 7.36-7.26 (m, 2H), 7.22 (d, J=7.4 Hz, 1H), 7.00-6.90 (m, 1H), 5.61 (s, 2H), 5.40-5.29 (m, 1H), 3.49 (s, 2H), 3.47 (s, 3H), 2.90-2.64 (m, 2H), 2.58-2.51 (m, 2H), 2.02-1.77 (m, 2H); MS (ES+): 502.20 (M+1); MS (ES−): 500.10 (M−1).

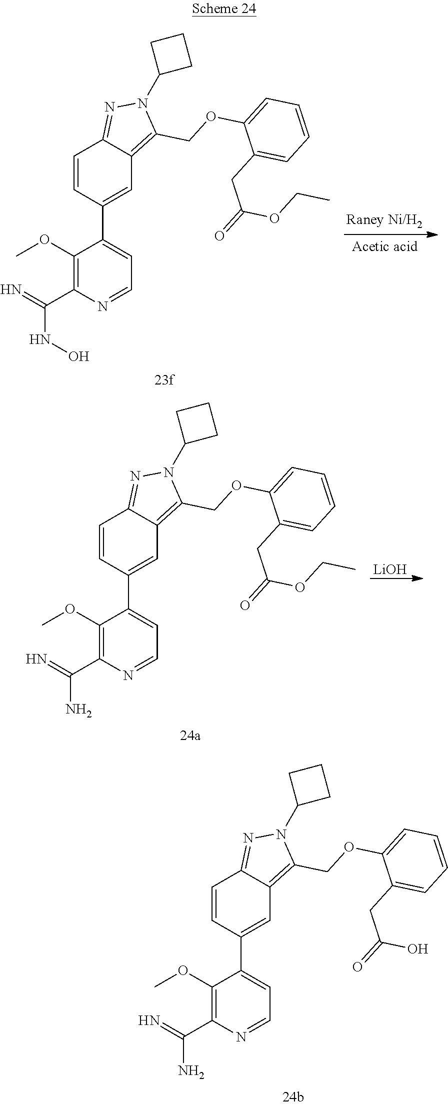

### Preparation of 2-(2-((5-(2-carbamimidoyl-3-methoxypyridin-4-yl)-2-cyclobutyl-2H-indazol-3-yl)methoxy)phenyl)acetic acid (24b)

### Step-1: Preparation of ethyl 2-(2-((5-(2-carbamimidoyl-3-methoxypyridin-4-yl)-2-cyclobutyl-2H-indazol-3-yl)methoxy)phenyl)acetate (24a)

To a solution of ethyl 2-(2-((2-cyclobutyl-5-(2-(N-hydroxycarbamimidoyl)-3-methoxypyridin-4-yl)-2H-indazol-3-yl)methoxy)phenyl)acetate (23f) (280 mg, 0.529 mmol) in acetic acid (0.030 mL, 0.529 mmol) and ethanol (5 mL) was added Raney nickel (0.529 mmol) and stirred under hydrogen atmosphere (balloon, 1 atm) for 16 h. The reaction mixture was filtered through a pad of Celite, washed with ethanol and the filtrate was concentrated in vacuo. The residue obtained was purified using method-E, to give ethyl 2-(2-((5-(2-carbamimidoyl-3-methoxypyridin-4-yl)-2-cyclobutyl-2H-indazol-3-yl)methoxy)phenyl)acetate (24a) (200 mg, 74% yield) as a colorless oil; 1H NMR (300 MHz, DMSO-d₆) δ 8.43 (d, J=4.8 Hz, 1H), 8.11 (s, 1H), 7.80 (d, J=9.0 Hz, 1H), 7.58-7.51 (m, 2H), 7.31 (d, J=5.1 Hz, 2H), 7.22 (d, J=7.4 Hz, 1H), 6.97 (d, J=7.2 Hz, 1H), 5.59 (s, 2H), 5.34 (p. J=8.2 Hz, 1H), 3.76 (q, J=7.1 Hz, 2H), 3.52 (s, 2H), 3.43 (s, 3H), 2.87-2.67 (m, 2H), 2.51-2.48 (m, 2H), 2.01-1.81 (m, 2H), 0.87 (t, J=7.1 Hz, 3H); MS (ES+): 514.20 (M+1).

### Step-2: Preparation of 2-(2-((5-(2-carbamimidoyl-3-methoxypyridin-4-yl)-2-cyclobutyl-2H-indazol-3-yl)methoxy)phenyl)acetic acid (24b)

Compound 24b was prepared according to the procedure and method of purification reported in step-2 of scheme 1, from ethyl 2-(2-((5-(2-carbamimidoyl-3-methoxypyridin-4-yl)-2-cyclobutyl-2H-indazol-3-yl)methoxy)phenyl)acetate (24a) (200 mg, 0.389 mmol) in MeOH/THF (6 mL) using a solution of LiOH·H₂O (98 mg, 2.337 mmol) in water (2 mL) to afford after workup and purification using method-G, 2-(2-((5-(2-carbamimidoyl-3-methoxypyridin-4-yl)-2-cyclobutyl-2H-indazol-3-yl)methoxy)phenyl)acetic acid (24b) (35 mg, 18.51% yield) HCl salt as a white solid; ¹H NMR (300 MHz, DMSO-d₆) δ 9.60-9.31 (m, 4H, D₂O exchangeable), 8.57 (d, J=4.8 Hz, 1H), 8.21 (s, 1H), 7.91-7.74 (m, 2H), 7.57 (d, J=9.0 Hz, 1H), 7.37-7.26 (m, 2H), 7.22 (d, J=7.4 Hz, 1H), 7.03-6.86 (m, 1H), 5.61 (s, 2H), 5.42-5.22 (m, 1H), 3.49 (s, 2H), 3.47 (s, 3H), 2.85-2.66 (m, 2H), 2.57-2.51 (m, 2H), 1.99-1.80 (m, 2H); MS (ES+): 486.20 (M+1); MS (ES−): 484.10 (M−1).

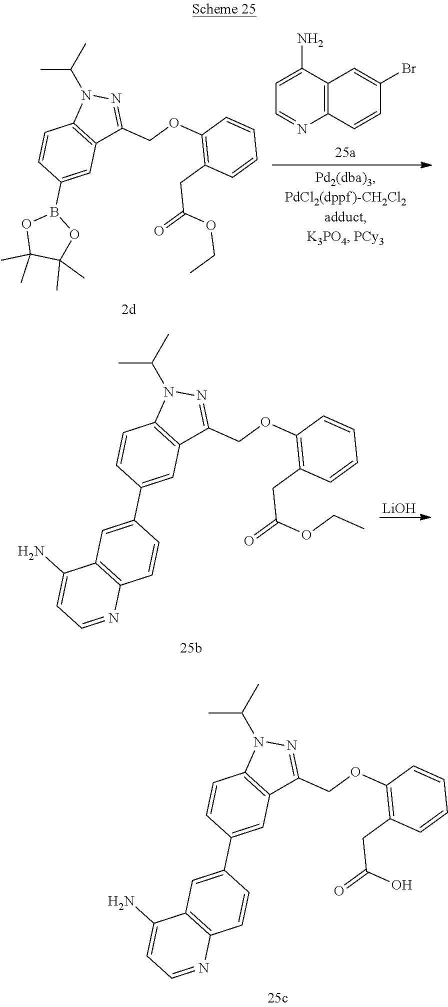

### Preparation of 2-(2-((5-(4-aminoquinolin-6-yl)-1-isopropyl-1H-indazol-3-yl)methoxy)phenyl)acetic acid (25c)

### Step-1: Preparation of ethyl 2-(2-((5-(4-aminoquinolin-6-yl)-1-isopropyl-1H-indazol-3-yl)methoxy)phenyl)acetate (25b)

Compound 25b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL) using added 6-bromoquinolin-4-amine (25a) (84 mg, 0.376 mmol; CAS #65340-73-0), K₃PO₄ (2M aqueous solution, 0.627 mL, 1.254 mmol). PCy₃ (17.59 mg, 0.063 mmol), Pd₂(dba)₃ (28.7 mg, 0.031 mmol) and PdCl₂(dppf)-CH₂Cl₂ adduct (25.6 mg, 0.031 mmol) to afford after workup and purification using method-E, ethyl 2-(2-((5-(4-aminoquinolin-6-yl)-1-isopropyl-1H-indazol-3-yl)methoxy)phenyl)acetate (25b) (100 mg, 64.5% yield) as a clear oil; ¹H NMR (300 MHz, DMSO-d₆) δ 8.48 (d, J=2.0 Hz, 1H), 8.29 (d, J=5.2 Hz, 1H), 8.14 (d, J=1.6 Hz, 1H), 8.01 (dd, J=8.9, 1.9 Hz, 1H), 7.94 (dd, J=8.9, 1.7 Hz, 1H), 7.83 (dd, J=14.6, 8.8 Hz, 2H), 7.32-7.28 (m, 2H), 7.19 (d, J=7.3 Hz, 1H), 6.98 (s, 2H), 6.95-6.87 (m, 1H), 6.56 (d, J=5.3 Hz, 1H), 5.45 (s, 2H), 5.07 (p. J=6.6 Hz, 1H), 3.63 (q, J=7.1 Hz, 2H), 3.53 (s, 2H), 1.53 (d, J=6.6 Hz, 6H), 0.72 (t, J=7.1 Hz, 3H); MS (ES+): 495.20 (M+1); MS (ES−): 493.10 (M−1).

### Step-2: Preparation of 2-(2-((5-(4-aminoquinolin-6-yl)-1-isopropyl-1H-indazol-3-yl)methoxy)phenyl)acetic acid (25c)

Compound 25c was prepared according to the procedure and method of purification reported in step-2 of scheme 1, from ethyl 2-(2-((5-(4-aminoquinolin-6-yl)-1-isopropyl-1H-indazol-3-yl)methoxy)phenyl)acetate (25b) (100 mg, 0.202 mmol) in THE/MeOH (4 mL) using a solution of LiOH·H₂O) (50.9 mg, 1.213 mmol) in water (1 mL) to afford after workup and purification using method-G, 2-(2-((5-(4-aminoquinolin-6-yl)-1-isopropyl-1H-indazol-3-yl)methoxy)phenyl)acetic acid (25c) (85 mg, 90% yield) HCl salt as a white solid; ¹H NMR (300 MHz, DMSO-d₆/D₂O) δ 8.78 (d, J=1.9 Hz, 1H), 8.45-8.32 (m, 2H), 8.23 (s, 1H), 8.05-7.82 (m, 3H), 7.33-7.24 (m, 2H), 7.19 (d, J=7.3 Hz, 1H), 6.97-6.87 (m, 1H), 6.82 (d, J=6.9 Hz, 1H), 5.46 (s, 2H), 5.15-4.97 (m, 1H), 3.52 (s, 2H), 1.52 (d, J=6.6 Hz, 6H); MS (ES+): 467.20 (M+1); MS (ES−): 465.15 (M−1).

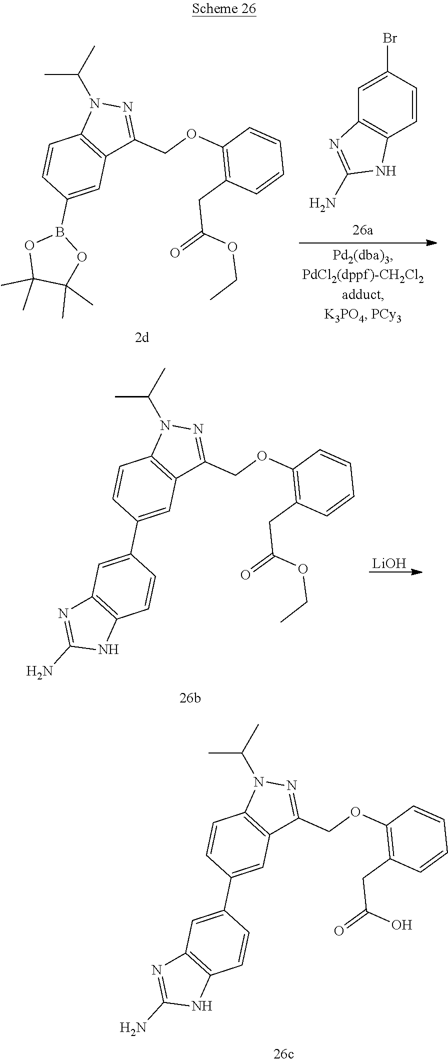 [SMILES: CCOC(=O)Cc1ccccc1OCc1nn(C(C)C)c2ccc(B3OC(C)(C)C(C)(C)O3)cc12 ; Nc1nc2cc(Br)ccc2[nH]1 ; CCOC(=O)Cc1ccccc1OCc1nn(C(C)C)c2ccc(-c3ccc4[nH]c(N)nc4c3)cc12 ; "LiOH" ; CC(C)n1nc(COc2ccccc2CC(=O)O)c2cc(-c3ccc4[nH]c(N)nc4c3)ccc21]

### Preparation of 2-(2-((5-(2-amino-1H-benzo[d]imidazol-5-yl)-1-isopropyl-1H-indazol-3-yl)methoxy)phenyl)acetic acid (26c)

### Step-1: Preparation of ethyl 2-(2-((5-(2-amino-1H-benzo[d]imidazol-5-yl)-1-isopropyl-1H-indazol-3-yl)methoxy)phenyl)acetate (26b)

Compound 26b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL) using 5-bromo-1H-benzo[d]imidazol-2-amine (26a) (177 mg, 0.836 mmol; CAS #791595-74-9), K₃PO₄ (2M aqueous solution, 0.836 mL, 1.672 mmol), PCy₃ (23.45 mg, 0.084 mmol). Pd₂(dba)₃ (38.3 mg, 0.042 mmol) and PdCl₂(dppf)-CH₂Cl₂ adduct (34.1 mg, 0.042 mmol) to afford after workup and purification using method-U, ethyl 2-(2-((5-(2-amino-1H-benzo[d]imidazol-5-yl)-1-isopropyl-1H-indazol-3-yl)methoxy)phenyl)acetate (26b) (50 mg, 24.73% yield) as a clear oil; MS (ES+): 484.20 (M+1); (ES−): 482.20 (M−1).

### Step-2: Preparation of 2-(2-((5-(2-amino-1H-benzo[d]imidazol-5-yl)-1-isopropyl-1H-indazol-3-yl)methoxy)phenyl)acetic acid (26c)

Compound 26c was prepared according to the procedure and method of purification reported in step-5 of scheme 2, from ethyl 2-(2-((5-(2-amino-1H-benzo[d]imidazol-5-yl)-1-isopropyl-1H-indazol-3-yl)methoxy)phenyl)acetate (26b) (50 mg, 0.103 mmol) in THF/MeOH (4 mL) using a solution of LiOH·H₂O (26.0 mg, 0.620 mmol) in water (1 mL) to afford after workup and purification using method-G, 2-(2-((5-(2-amino-1H-benzo[d]imidazol-5-yl)-1-isopropyl-1H-indazol-3-yl)methoxy)phenyl)acetic acid (26c) (7 mg, 14.86% yield) HCl salt as a white solid; 1H NMR (300 MHz, DMSO-d₆) δ 8.43 (s, 2H, D₂O exchangeable), 7.99 (s, 1H), 7.81 (d, J=8.9 Hz, 1H), 7.69 (dd, J=8.9, 1.6 Hz, 1H), 7.60 (s, 1H), 7.58-7.50 (m, 1H), 7.42 (d, J=8.3 Hz, 1H), 7.32-7.23 (m, 2H), 7.20 (d, J=7.4 Hz, 1H), 6.97-6.85 (m, 1H), 5.46 (s, 2H), 5.11-4.92 (m, 1H), 3.52 (s, 2H), 1.52 (d, J=6.5 Hz, 6H); MS (ES+): 456.20 (M+1); (ES−): 454.10 (M−1).

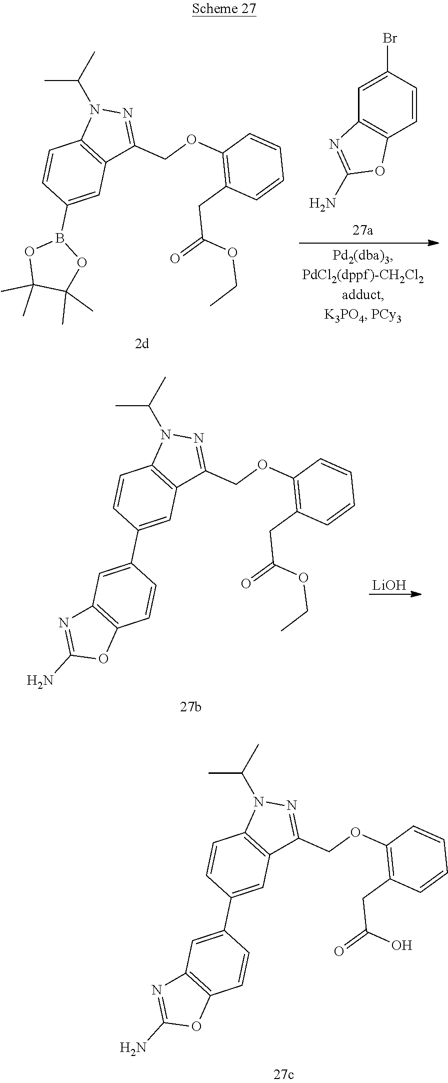 [SMILES: CCOC(=O)Cc1ccccc1OCc1nn(C(C)C)c2ccc(B3OC(C)(C)C(C)(C)O3)cc12 ; Nc1nc2cc(Br)ccc2o1 ; CCOC(=O)Cc1ccccc1OCc1nn(C(C)C)c2ccc(-c3ccc4oc(N)nc4c3)cc12 ; "LiOH" ; CC(C)n1nc(COc2ccccc2CC(=O)O)c2cc(-c3ccc4oc(N)nc4c3)ccc21]

### Preparation of 2-(2-((5-(2-aminobenzo[d]oxazol-5-yl)-1-isopropyl-1H-indazol-3-yl)methoxy)phenyl)acetic acid (27c)

### Step-1: Preparation of ethyl 2-(2-((5-(2-aminobenzo[d]oxazol-5-yl)-1-isopropyl-1H-indazol-3-yl)methoxy)phenyl)acetate (27b)

Compound 27b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL) using 5-bromobenzo[d]oxazol-2-amine (27a) (178 mg, 0.836 mmol; CAS #64037-07-6), K₃PO₄ (2M aqueous solution, 0.836 mL, 1.672 mmol). PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-U, ethyl 2-(2-((5-(2-aminobenzo[d]oxazol-5-yl)-1-isopropyl-1H-indazol-3-yl)methoxy)phenyl)acetate (27b) (150 mg, 74.0% yield) as a clear oil; ¹H NMR (300 MHz, DMSO-d₆) δ 7.93 (d, J=1.5 Hz, 1H), 7.76 (d, J=8.8 Hz, 1H), 7.69 (dd, J=8.8, 1.6 Hz, 1H), 7.48 (d, J=1.7 Hz, 2H), 7.38 (d, J=8.2 Hz, 1H), 7.28 (dd, J=3.5, 1.9 Hz, 2H), 7.19 (d, J=7.3 Hz, 1H), 6.91 (dt, J=7.8, 4.2 Hz, 1H), 5.42 (s, 2H), 5.02 (p, J=6.6 Hz, 1H), 3.67 (q, J=7.1 Hz, 2H), 3.52 (s, 2H), 1.51 (d, J=6.6 Hz, 6H), 0.77 (t, J=7.1 Hz, 3H); MS (ES+): 485.20 (M+1); 507.20 (M+Na); (ES−): 483.20 (M−1).

### Step-2: Preparation of 2-(2-((5-(2-aminobenzo[d]oxazol-5-yl)-1-isopropyl-1H-indazol-3-yl)methoxy)phenyl)acetic acid (27c)

Compound 27c was prepared according to the procedure and method of purification reported in step-2 of scheme 1, from ethyl 2-(2-((5-(2-aminobenzo[d]oxazol-5-yl)-1-isopropyl-1H-indazol-3-yl)methoxy)phenyl)acetate (27b) (150 mg, 0.310 mmol) in THF/MeOH (4 mL) using a solution of LiOH·H₂O (78 mg, 1.857 mmol) in water (1 mL) to afford after workup and purification using method-G, 2-(2-((5-(2-aminobenzo[d]oxazol-5-yl)-1-isopropyl-1H-indazol-3-yl)methoxy)phenyl)acetic acid (27c) (60 mg, 0.131 mmol, 42.5% yield) HCl salt as a white solid; ¹H NMR (300 MHz, DMSO-d₆) δ 8.55 (s, 2H, D₂O exchangeable), 8.01 (s, 1H), 7.79 (d, J=8.9 Hz, 1H), 7.70 (dd, J=8.9, 1.6 Hz, 1H), 7.60-7.47 (m, 2H), 7.47-7.37 (m, 1H), 7.34-7.22 (m, 2H), 7.22-7.15 (m, 1H), 6.97-6.85 (m, 1H), 5.46 (s, 2H), 5.10-4.95 (m, 1H), 3.52 (s, 2H), 1.51 (d, J=6.6 Hz, 6H); MS (ES+): 457.20 (M+1); (ES−): 455.10 (M−1).

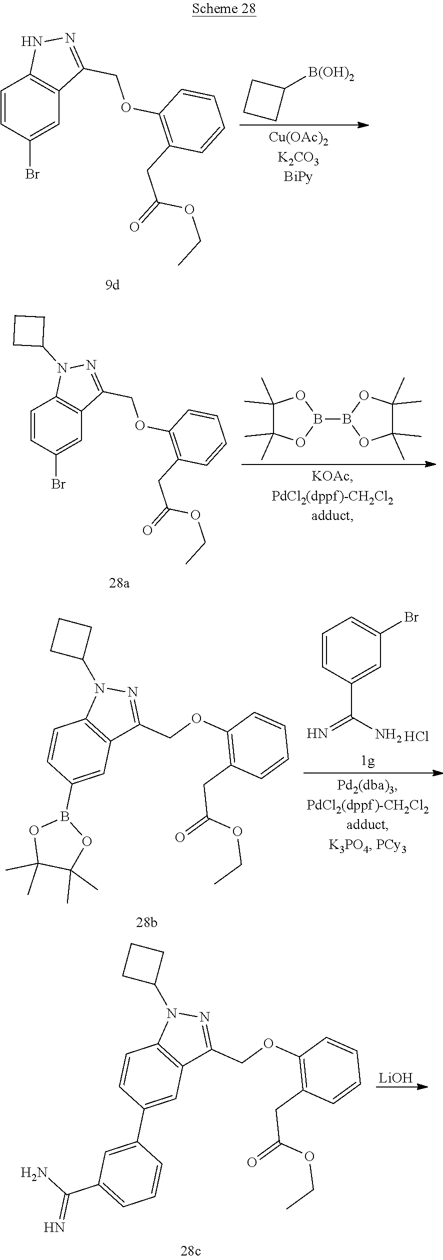

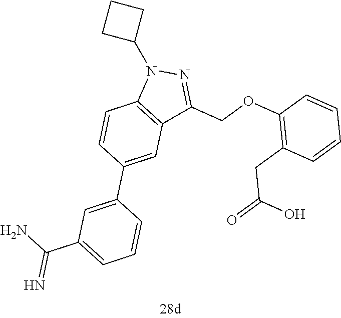 [SMILES: N=C(N)c1cccc(-c2ccc3c(c2)c(COc2ccccc2CC(=O)O)nn3C2CCC2)c1]

### Preparation of 2-(2-((5-(3-carbamimidoylphenyl)-1-cyclobutyl-1H-indazol-3-yl)methoxy)phenyl)acetic acid (28d)

### Step-1: Preparation of ethyl 2-(2-((5-bromo-1-cyclobutyl-1H-indazol-3-yl)methoxy)phenyl)acetate (28a)

To a solution of ethyl 2-(2-((5-bromo-1H-indazol-3-yl)methoxy)phenyl)acetate (9d) (1 g, 2.57 mmol) in DCE (20 mL) was added cyclobutyl boronic acid (0.385 g, 3.85 mmol; CAS #849052-26-2), 2,2′-bipyridine (0.100 g, 0.642 mmol), copper (II) acetate (0.467 g, 2.57 mmol) and potassium carbonate (0.71 g, 5.14 mmol) and heated at 80° ° C. for 3 h. The reaction mixture was filtered through a pad of celite and the filtrate was concentrated in vacuo. The residue obtained was purified using Method-P, to give ethyl 2-(2-((5-bromo-1-cyclobutyl-1H-indazol-3-yl)methoxy)phenyl)acetate (28a) (400 mg, 35.1% yield) as a clear oil; 1H NMR (300 MHz, DMSO-d₆) δ 7.94 (d, J=1.8 Hz, 1H), 7.70 (d, J=8.9 Hz, 1H), 7.52 (dd, J=8.9, 1.9 Hz, 1H), 7.34-7.22 (m, 2H), 7.20 (dd, J=7.3, 1.5 Hz, 1H), 6.92 (td, J=7.1, 1.7 Hz, 1H), 5.39 (s, 2H), 5.29 (p, J=8.3 Hz, 1H), 3.91 (q. J=7.1 Hz, 2H), 3.53 (s, 2H), 2.62 (pd, J=9.4, 2.7 Hz, 2H), 2.48-2.36 (m, 2H), 1.87 (m, 2H), 0.93 (t, J=7.1 Hz, 3H); MS (ES+): 443.10 & 445.10 (M+1); (ES−): 441.00 (M−1).

### Step-2: Preparation of ethyl 2-(2-((1-cyclobutyl-5-(4,4,5,5-tetramethyl-1,3,2-dioxaborolan-2-yl)-1H-indazol-3-yl)methoxy)phenyl)acetate (28b)

Compound 28b was prepared according to the procedure and method of purification reported in step-4 of scheme 1, from ethyl 2-(2-((5-bromo-1-cyclobutyl-1H-indazol-3-yl)methoxy)phenyl)acetate (28a) (400 mg, 0.902 mmol) in anhydrous dioxane (20 mL), using BISPIN (458 mg, 1.805 mmol), KOAc (221 mg, 2.256 mmol) and PdCl₂dppf)-CH₂Cl₂ adduct (44.2 mg, 0.054 mmol) to afford after workup and purification using method-C, ethyl 2-(2-((1-cyclobutyl-5-(4,4,5,5-tetramethyl-1,3,2-dioxaborolan-2-yl)-1H-indazol-3-yl)methoxy)phenyl)acetate (28b) (350 mg, 79% yield) as a clear oil; 1H NMR (300 MHz, DMSO-d₆) δ 8.15 (d, J=1.0 Hz, 1H), 7.77-7.58 (m, 2H), 7.34-7.23 (m, 2H), 7.23-7.15 (m, 1H), 6.93 (ddt, J=7.4, 5.2, 3.1 Hz, 1H), 5.42 (s, 2H), 5.29 (p, J=8.3 Hz, 1H), 3.85 (q. J=7.1 Hz, 2H), 3.53 (s, 2H), 2.63 (b, J=6.7, 4.4 Hz, 2H), 2.49-2.39 (m, 2H), 1.88 (td, J=10.7, 10.1, 4.1 Hz, 2H), 1.29 (s, 9H), 1.16 (s, 3H), 0.87 (t, J=7.1 Hz, 3H); MS (ES+): 491.30 (M+1); 513.30 (M+Na); (ES−): 489.00 (M−1).

### Step-3: Preparation of ethyl 2-(2-((5-(3-carbamimidoylphenyl)-1-cyclobutyl-1H-indazol-3-yl)methoxy)phenyl)acetate (28c)

Compound 28c was prepared according to the procedure and method of purification reported in step-5 of scheme 1, from 2-(2-((1-cyclobutyl-5-(4,4,5,5-tetramethyl-1,3,2-dioxaborolan-2-yl)-1H-indazol-3-yl)methoxy)phenyl)acetate (28b) (350 mg, 0.714 mmol) in dioxane/THF (6 mL, 1:1) using 3-bromobenzimidamide hydrochloride (1g) (336 mg, 1.427 mmol), K₃PO₄ (2M aqueous solution, 1.427 mL, 2.85 mmol), PCy₃ (40.0 mg, 0.143 mmol), Pd₂(dba)₃ (65.4 mg, 0.071 mmol) and PdCl₂(dppf)-CH₂Cl₂ adduct (58.3 mg, 0.071 mmol) to afford after workup and purification using method-F, ethyl 2-(2-((5-(3-carbamimidoylphenyl)-1-cyclobutyl-1H-indazol-3-yl)methoxy)phenyl)acetate (28c) (200 mg, 58.1% yield) as a clear oil; MS (ES+): 483.30 (M+1).

### Step-4: Preparation of 2-(2-((5-(3-carbamimidoylphenyl)-1-cyclobutyl-1H-indazol-3-yl)methoxy)phenyl)acetic acid (28d)

Compound 28d was prepared according to the procedure and method of purification reported in step-2 of scheme 1, from ethyl 2-(2-((5-(3-carbamimidoylphenyl)-1-cyclobutyl-1H-indazol-3-yl)methoxy)phenyl)acetate (28c) (200 mg, 0.414 mmol) in THE/MeOH (4 mL) using a solution of LiOH·H₂O (104 mg, 2.487 mmol) in water (1 mL) to afford after workup and purification using method-G, 2-(2-((5-(3-carbamimidoylphenyl)-1-cyclobutyl-1H-indazol-3-yl)methoxy)phenyl)acetic acid (28d) (36 mg, 19.11% yield) HCl salt as a white solid; ¹H NMR (300 MHz, DMSO-d₆) δ 12.07 (s, 1H, D₂O exchangeable), 9.44 (s, 2H, D₂O exchangeable), 9.15 (s, 2H, D₂O exchangeable), 8.19 (s, 1H), 8.17-8.12 (m, 1H), 8.12-8.04 (m, 1H), 7.93-7.81 (m, 2H), 7.80-7.74 (m, 1H), 7.70 (t, J=7.7 Hz, 1H), 7.34-7.23 (m, 2H), 7.23-7.16 (m, 1H), 6.97-6.87 (m, 1H), 5.48 (s, 2H), 5.42-5.27 (m, 1H), 3.53 (s, 2H), 2.78-2.57 (m, 2H), 2.49-2.44 (m, 2H), 2.00-1.80 (m, 2H); MS (ES+): 455.20 (M+1); (ES−): 453.20 (M−1); Analysis calculated for C₂/H₂₆NO₃·HCl·1.75H₂O: C, 62.06; H, 5.88; N, 10.72; Found: C, 62.16; H, 5.71; N, 10.76.

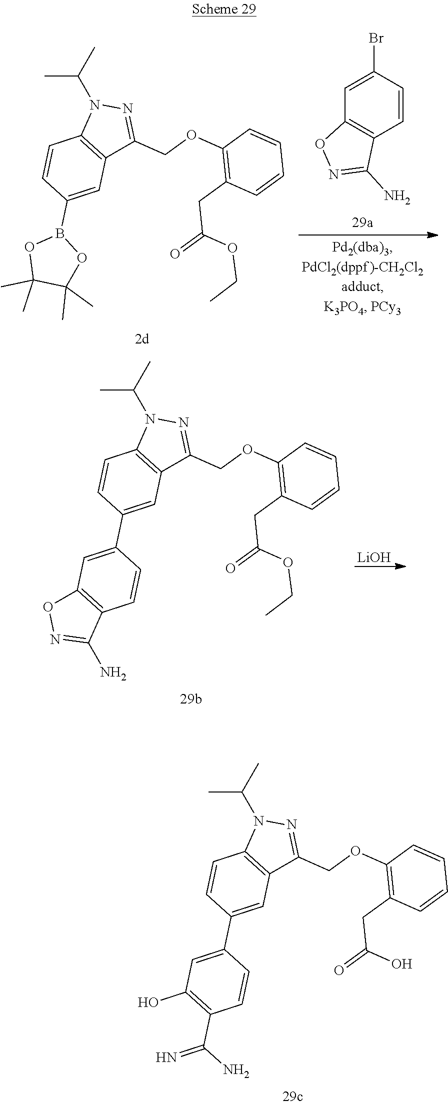

### Preparation of 2-(2-((5-(4-carbamimidoyl-3-hydroxyphenyl)-1-isopropyl-1H-indazol-3-yl)methoxy)phenyl)acetic acid (29c)

### Step-1: Preparation of ethyl 2-(2-((5-(3-aminobenzo[d]isoxazol-6-yl)-1-isopropyl-1H-indazol-3-yl)methoxy)phenyl)acetate (29b)

Compound 29b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 6-bromobenzo[d]isoxazol-3-amine (29a) (178 mg, 0.836 mmol), K₃PO₄ (2M aqueous solution, 0.836 mL, 1.672 mmol), PCy₃ (23.45 mg, 0.084 mmol), Pd₂(dba)₃ (38.3 mg, 0.042 mmol), and PdCl₂(dppf)-CH₂Cl₂ adduct (34.1 mg, 0.042 mmol) to afford after workup and purification using method-U, ethyl 2-(2-((5-(3-aminobenzo[d]isoxazol-6-yl)-1-isopropyl-1H-indazol-3-yl)methoxy)phenyl)acetate (29b) as a clear oil; MS (ES+): 485.20 (M+1); 507.20 (M+Na); (ES−): 483.05 (M−1).

### Step-2: Preparation of 2-(2-((5-(4-carbamimidoyl-3-hydroxyphenyl)-1-isopropyl-1H-indazol-3-yl)methoxy)phenyl)acetic acid (29c)

Compound 29c was prepared according to the procedure and method of purification reported in step-5 of scheme 2, from ethyl 2-(2-((5-(3-aminobenzo[d]isoxazol-6-yl)-1-isopropyl-1H-indazol-3-yl)methoxy)phenyl)acetate (29b) (100 mg, 0.206 mmol) in THE/MeOH (4 mL) using a solution of LiOH·H₂O (52.0 mg, 1.238 mmol) in water (1 mL) to afford after workup and purification using method-G, 2-(2-((5-(4-carbamimidoyl-3-hydroxyphenyl)-1-isopropyl-1H-indazol-3-yl)methoxy)phenyl)acetic acid (29c) (12 mg, 0.026 mmol, 12.68% yield) HCl salt as a white solid; 1H NMR (300 MHz, DMSO-d₆) δ 12.06 (s, 1H, D₂O exchangeable), 11.21 (s, 1H, D₂O exchangeable), 9.04 (s, 2H, D₂O exchangeable), 8.83 (s, 2H, D₂O exchangeable), 8.06 (d, J=1.6 Hz, 1H), 7.85 (d, J=8.9 Hz, 1H), 7.71-7.61 (m, 2H), 7.40-7.31 (m, 2H), 7.31-7.24 (m, 2H), 7.20 (d, J=7.3 Hz, 1H), 6.97-6.87 (m, 1H), 5.47 (s, 2H), 5.16-4.92 (m, 1H), 3.51 (s, 2H), 1.51 (d, J=6.6 Hz, 6H); MS (ES+): 459.20 (M+1); (ES−): 457.20 (M−1).

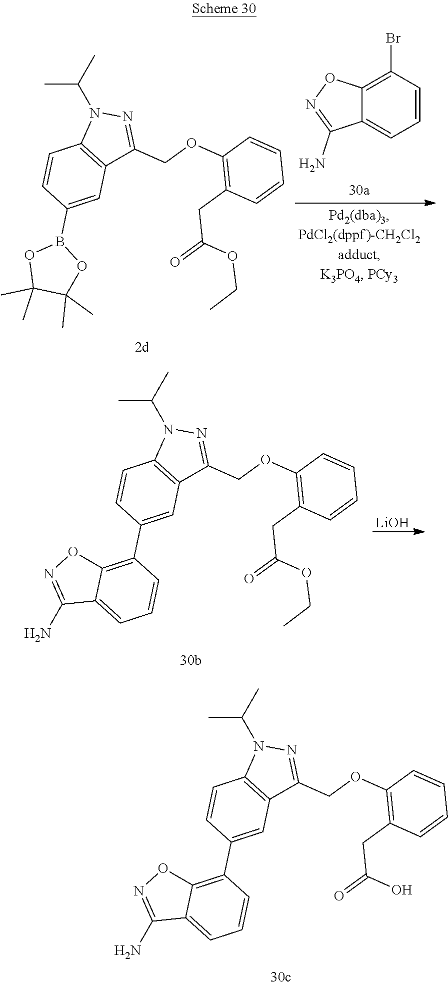

### Preparation of 2-(2-((5-(3-aminobenzo[d]isoxazol-7-yl)-1-isopropyl-1H-indazol-3-yl)methoxy)phenyl)acetic acid (30c)

### Step-1: Preparation of ethyl 2-(2-((5-(3-aminobenzo[d]isoxazol-7-yl)-1-isopropyl-1H-indazol-3-yl)methoxy)phenyl)acetate (30b)

Compound 30b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 7-bromobenzo[d]isoxazol-3-amine (30a) (178 mg, 0.836 mmol; CAS #1260860-32-9), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-U, ethyl 2-(2-((5-(3-aminobenzo[d]isoxazol-7-yl)-1-isopropyl-1H-indazol-3-yl)methoxy)phenyl)acetate (30b) (50 mg, 24.68% yield) as a clear oil; ¹H NMR (300 MHz, DMSO-d₆) δ 8.28-8.12 (m, 1H), 7.94-7.85 (m, 2H), 7.85-7.76 (m, 2H), 7.35 (t, J=7.6 Hz, 1H), 7.31-7.24 (m, 2H), 7.18 (d, J=7.3 Hz, 1H), 6.97-6.82 (m, 1H), 6.48 (s, 2H), 5.45 (s, 2H), 5.06 (p, J=6.5 Hz, 1H), 3.60 (q, J=7.1 Hz, 2H), 3.55 (s, 2H), 1.53 (d, J=6.6 Hz, 6H), 0.72 (t, J=7.1 Hz, 3H); MS (ES+): 485.20 (M+1); 507.20 (M+Na); (ES−): 483.20 (M−1).

### Step-2: Preparation of 2-(2-((5-(3-aminobenzo[d]isoxazol-7-yl)-1-isopropyl-1H-indazol-3-yl)methoxy)phenyl)acetic acid (30c)

Compound 30c was prepared according to the procedure and method of purification reported in step-2 of scheme 1, from ethyl 2-(2-((5-(3-aminobenzo[d]isoxazol-7-yl)-1-isopropyl-1H-indazol-3-yl)methoxy)phenyl)acetate (30b) (50 mg, 0.103 mmol) in THF/MeOH (4 mL, 1:1) using a solution of LiOH·H₂O (26.0 mg, 0.619 mmol) in water (1 mL) to afford after workup and purification using method-G, 2-(2-((5-(3-aminobenzo[d]isoxazol-7-yl)-1-isopropyl-1H-indazol-3-yl)methoxy)phenyl)acetic acid (30c) (8 mg, 16.98% yield) HCl salt as a white solid; ¹H NMR (300 MHz, DMSO-d₆) δ 8.28 (d, J=1.5 Hz, 1H), 7.93 (dd, J=8.9, 1.6 Hz, JH), 7.88-7.76 (m, 3H), 7.36 (t, J=7.6 Hz. JH), 7.31-7.22 (m, 2H), 7.22-7.14 (m, 1H), 6.96-6.85 (m, 1H), 5.47 (s, 2H), 5.18-4.90 (m, 1H), 3.56 (s, 2H), 1.53 (d, J=6.6 Hz, 6H); MS (ES+): 457.20 (M+1); (ES−): 455.10 (M−1).

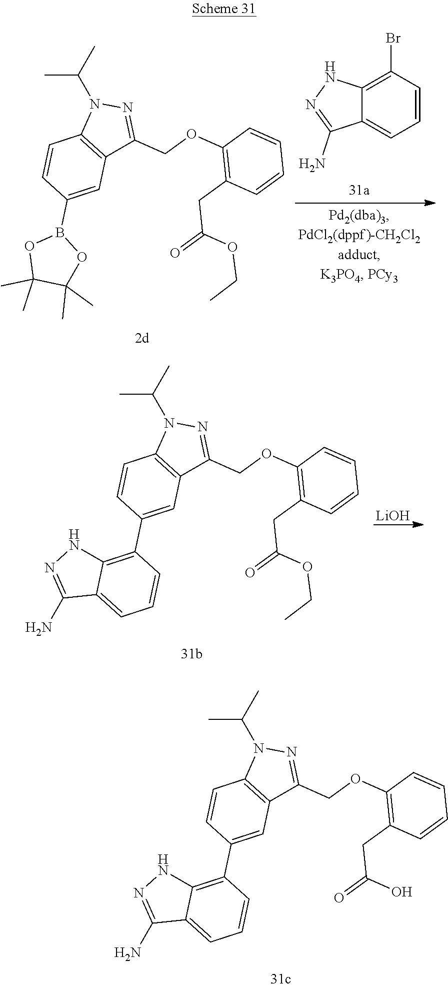

### Preparation of 2-(2-((3′-amino-1-isopropyl-1H, 1′H-[5,7′-biindazol]-3-yl)methoxy)phenyl)acetic acid (31c)

### Step-1: Preparation of ethyl 2-(2-((3′-amino-1-isopropyl-1H, 1′H-[5,7′-biindazol]-3-yl)methoxy)phenyl)acetate (31b)

Compound 31b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 7-bromo-1H-indazol-3-amine (31a) (177 mg, 0.836 mmol; CAS #1234616-28-4), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-R, ethyl 2-(2-((3′-amino-1-isopropyl-1H, 1H-[5,7′-biindazol]-3-yl)methoxy)phenyl)acetate (31b) (150 mg, 74.2% yield) as a clear oil; ¹H NMR (300 MHz, DMSO-d₆) δ 11.76 (s, 1H), 11.52 (s, 1H), 8.03 (s, 1H), 7.82 (d, J=8.8 Hz, 1H), 7.75-7.71 (m, 1H), 7.48-7.41 (m, 1H), 7.38-7.32 (m, 1H), 7.31-7.26 (m, 2H), 7.19-7.12 (m, 1H), 7.02 (t, J=7.5 Hz, 1H), 6.84 (t, J=7.8 Hz, 1H), 5.46 (s, 2H), 5.05 (p. J=6.6 Hz, 1H), 3.95 (s, 2H), 3.63-3.57 (m, 2H), 1.53 (d, J=6.6 Hz, 6H), 0.67 (t, J=7.1 Hz, 3H); MS (ES+): 484.20 (M+1); (ES−): 482.10 (M−1).

### Step-2: Preparation of 2-(2-((3′-amino-1-isopropyl-1H, 1′H-[5,7′-biindazol]-3-yl)methoxy)phenyl)acetic acid (31c)

Compound 31c was prepared according to the procedure and method of purification reported in step-2 of scheme 1, from ethyl 2-(2-((3′-amino-1-isopropyl-1H, 1H-[5,7′-biindazol]-3-yl)methoxy)phenyl)acetate (31b) (150 mg, 0.310 mmol) in THE/MeOH (4 mL, 1:1) using a solution of LiOH·H₂O (78 mg, 1.861 mmol) in water (1 mL) to afford after workup and purification using method-G, 2-(2-((3′-amino-1-isopropyl-1H, 1′H-[5,7′-biindazol]-3-yl)methoxy)phenyl)acetic acid (31c) (65 mg, 0.143 mmol, 46.0% yield) HCl salt as a white solid; 1H NMR (300 MHz, DMSO-d₆) δ 8.10 (s, 1H), 8.00 (d, J=8.1 Hz, 1H), 7.88 (d, J=8.8 Hz, 1H), 7.76-7.62 (m, 2H), 7.36-7.23 (m, 3H), 7.23-7.13 (m, 1H), 6.90 (td, J=7.2, 1.6 Hz, 1H), 5.48 (s, 2H), 5.21-4.87 (m, 1H), 3.49 (s, 2H), 1.53 (d, J=6.5 Hz, 6H); MS (ES+): 456.15 (M+1); (ES−): 454.15 (M−1).

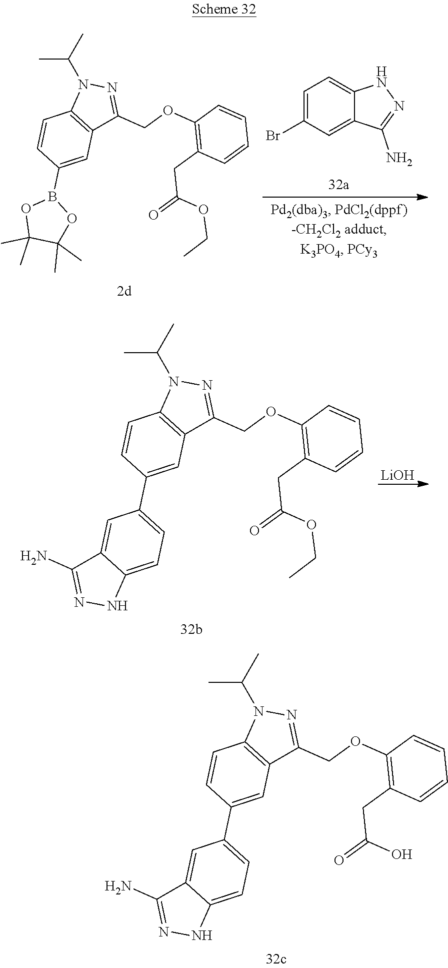

### Preparation of 2-(2-((3′-amino-1-isopropyl-1H, 1′H-[5,5′-biindazol]-3-yl)methoxy)phenyl)acetic acid (32c)

### Step-1: Preparation of ethyl 2-(2-((3′-amino-1-isopropyl-1H, 1′H-[5,5′-biindazol]-3-yl)methoxy)phenyl)acetate (32b)

Compound 32b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 5-bromo-1H-indazol-3-amine (32a) (177 mg, 0.836 mmol; CAS #61272-71-7), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-R, ethyl 2-(2-((3′-amino-1-isopropyl-1H, 1H-[5,5′-biindazol]-3-yl)methoxy)phenyl)acetate (160 mg, 79% yield) as a clear oil; JH NMR (300 MHz, DMSO-d₆) δ 11.58 (s, 1H), 11.46-11.32 (m, 1H), 8.06-7.89 (m, 2H), 7.84-7.51 (m, 2H), 7.35-7.25 (m, 4H), 7.24-7.16 (m, 2H), 6.92 (d, J=7.6 Hz, 1H), 5.42 (s, 2H), 5.01 (q, J=6.7 Hz, 1H), 3.95 (s, 2H), 3.68-3.55 (m, 2H), 1.52 (d, J=6.5 Hz, 6H), 0.74 (t, J=7.1 Hz, 3H); MS (ES+): 484.20 (M+1).

### Step-2: Preparation of 2-(2-((3′-amino-1-isopropyl-1H, l′H-[5,5′-biindazol]-3-yl)methoxy)phenyl)acetic acid (32c)

Compound 32c was prepared according to the procedure and method of purification reported in step-2 of scheme 1, from ethyl 2-(2-((3′-amino-1-isopropyl-1H, 1H-[5,5′-biindazol]-3-yl)methoxy)phenyl)acetate (32b) (160 mg, 0.331 mmol) in THF/MeOH (4 mL) using a solution of LiOH·H₂O (83 mg, 1.985 mmol) in water (1 mL) to afford after workup and purification using method-G, 2-(2-((3′-amino-1-isopropyl-1H, 1′H-[5,5′-biindazol]-3-yl)methoxy)phenyl)acetic acid (32c) (35 mg, 0.077 mmol, 23.22% yield) HCl salt as a white solid; 1H NMR (300 MHz, DMSO-d₆) δ 8.23 (s, 1H), 8.03 (s, 1H), 7.93-7.78 (m, 2H), 7.72 (dd, J=8.8, 1.7 Hz, 1H), 7.49 (d, J=8.8 Hz, 1H), 7.32-7.23 (m, 2H), 7.20 (d, J=7.3 Hz, 1H), 6.96-6.88 (m, 1H), 5.45 (s, 2H), 5.11-4.94 (m, 1H), 3.52 (s, 2H), 1.52 (d, J=6.6 Hz, 6H); MS (ES+): 456.20 (M+1); (ES−): 454.20 (M−1).

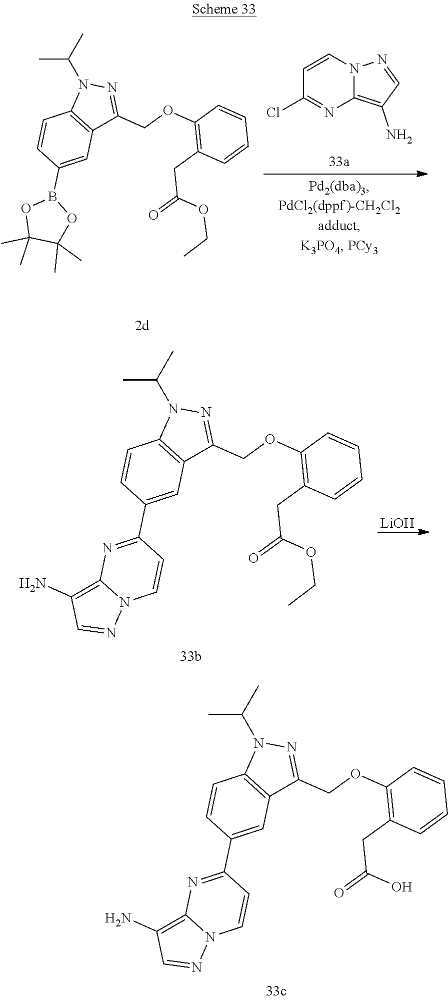

### Preparation of 2-(2-((5-(3-aminopyrazolo[1,5-a]pyrimidin-5-yl)-1-isopropyl-1H-indazol-3-yl)methoxy)phenyl)acetic acid (33c)

### Step-1: Preparation of ethyl 2-(2-((5-(3-aminopyrazolo[1,5-a]pyrimidin-5-yl)-1-isopropyl-1H-indazol-3-yl)methoxy)phenyl)acetate (33b)

Compound 33b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 5-chloropyrazolo[1,5-a]pyrimidin-3-amine (33a) (141 mg, 0.836 mmol; CAS #1234616-50-2), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-R, ethyl 2-(2-((5-(3-aminopyrazolo[1,5-a]pyrimidin-5-yl)-1-isopropyl-1H-indazol-3-yl)methoxy)phenyl)acetate (33b) (120 mg, 59.2% yield) as a clear oil; ¹H NMR (300 MHz, DMSO-d₆) δ 8.82 (d, J=7.5 Hz, 1H), 8.54 (s, 1H), 8.36 (dd, J=9.0, 1.6 Hz, 1H), 7.85 (d, J=9.0 Hz, 1H), 7.71 (s, 1H), 7.44 (d, J=7.6 Hz, 1H), 7.30 (d, J=3.9 Hz, 2H), 7.21 (d, J=7.3 Hz, 1H), 6.98-6.87 (m, 1H), 5.45 (s, 2H), 5.06 (p. J=6.6 Hz, 1H), 4.42 (s, 2H), 3.76 (q, J=7.1 Hz, 2H), 3.56 (s, 2H), 1.52 (d, J=6.6 Hz, 6H), 0.77 (t, J=7.1 Hz, 3H); MS (ES+): 485.20 (M+1); 507.25 (M+Na).

### Step-2: Preparation of 2-(2-((5-(3-aminopyrazolo[1,5-a]pyrimidin-5-yl)-1-isopropyl-1H-indazol-3-yl)methoxy)phenyl)acetic acid (33c)

Compound 33c was prepared according to the procedure and method of purification reported in step-2 of scheme 1, from ethyl 2-(2-((5-(3-aminopyrazolo[1,5-a]pyrimidin-5-yl)-1-isopropyl-1H-indazol-3-yl)methoxy)phenyl)acetate (33b) (120 mg, 0.248 mmol) in THF/MeOH (4 mL) using a solution of LiOH·H₂O (62.4 mg, 1.486 mmol) in water (1 mL) to afford after workup and purification using method-G, 2-(2-((5-(3-aminopyrazolo[1,5-a]pyrimidin-5-yl)-1-isopropyl-1H-indazol-3-yl)methoxy)phenyl)acetic acid (33c) HCl salt as a white solid; ¹H NMR (300 MHz, DMSO-d₆) ¿ 10.55 (s, 3H, D₂O exchangeable), 9.16 (d, J=7.5 Hz, 1H), 8.71 (d, J=1.6 Hz, 1H), 8.43 (dd, J=9.0, 1.6 Hz, 1H), 8.27 (s, 1H), 7.93 (d, J=9.0 Hz, 1H), 7.84 (d, J=7.6 Hz, 1H), 7.32-7.25 (m, 2H), 7.22 (d, J=7.3 Hz, 1H), 6.99-6.86 (m, 1H), 5.48 (s, 2H), 5.25-4.80 (m, 1H), 3.55 (s, 2H), 1.53 (d, J=6.6 Hz, 6H); MS (ES+): 457.20 (M+1); (ES−): 455.20 (M−1).

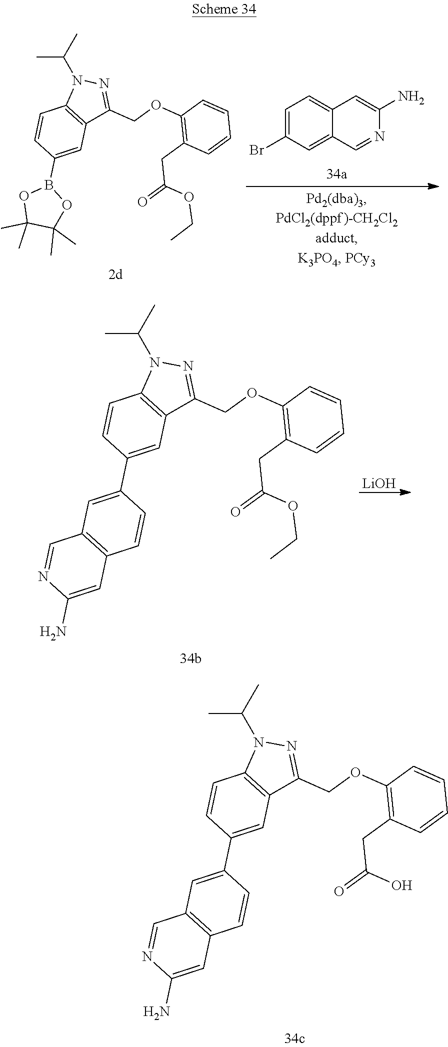

### Preparation of 2-(2-((5-(3-aminoisoquinolin-7-yl)-1-isopropyl-1H-indazol-3-yl)methoxy)phenyl)acetic acid (34c)

### Step-1: Preparation of ethyl 2-(2-((5-(3-aminoisoquinolin-7-yl)-1-isopropyl-1H-indazol-3-yl)methoxy)phenyl)acetate (34b)

Compound 34b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 7-bromoisoquinolin-3-amine (34a) (187 mg, 0.836 mmol; CAS #1192815-02-3), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-R, ethyl 2-(2-((5-(3-aminoisoquinolin-7-yl)-1-isopropyl-1H-indazol-3-yl)methoxy)phenyl)acetate (34b) (150 mg, 72.5% yield) as a clear oil; 1H NMR (300 MHz, DMSO-d₆) δ 8.88 (s, 1H), 8.16-8.01 (m, 2H), 7.90-7.78 (m, 3H), 7.61 (d, J=8.7 Hz, 1H), 7.33-7.26 (m, 2H), 7.23-7.13 (m, 1H), 6.98-6.85 (m, 1H), 6.64 (s, 1H), 5.98 (s, 2H), 5.44 (s, 2H), 5.04 (p, J=6.6 Hz, 1H), 3.68-3.62 (m, 2H), 3.54 (s, 2H), 1.52 (d, J=6.6 Hz, 6H), 0.73 (t, J=7.1 Hz, 3H); MS (ES+): 495.25 (M+1).

### Step-2: Preparation of 2-(2-((5-(3-aminoisoquinolin-7-yl)-1-isopropyl-1H-indazol-3-yl)methoxy)phenyl)acetic acid (34c)

Compound 34c was prepared according to the procedure and method of purification reported in step-2 of scheme 1, from ethyl 2-(2-((5-(3-aminoisoquinolin-7-yl)-1-isopropyl-1H-indazol-3-yl)methoxy)phenyl)acetate (34b) (150 mg, 0.303 mmol) in THE/MeOH (4 mL, 1:1) using a solution of LiOH·H₂O (76 mg, 1.82 mmol) in water (1 mL) to afford after workup and purification using method-G, 2-(2-((5-(3-aminoisoquinolin-7-yl)-1-isopropyl-1H-indazol-3-yl)methoxy)phenyl)acetic acid (34c) HCl salt as a white solid; ¹H NMR (300 MHz, DMSO-d₆) δ 9.13 (s, 1H), 8.34 (s, 1H), 8.20-8.13 (m, 2H), 7.91-7.82 (m, 3H), 7.29-7.26 (m, 2H), 7.23-7.21 (m, 2H), 6.97-6.87 (m, 1H), 5.47 (s, 2H), 5.11-4.98 (m, 1H), 3.55 (s, 2H), 1.52 (d, J=6.6 Hz, 6H); MS (ES+): 467.20 (M+1); (ES−): 465.10 (M−1).

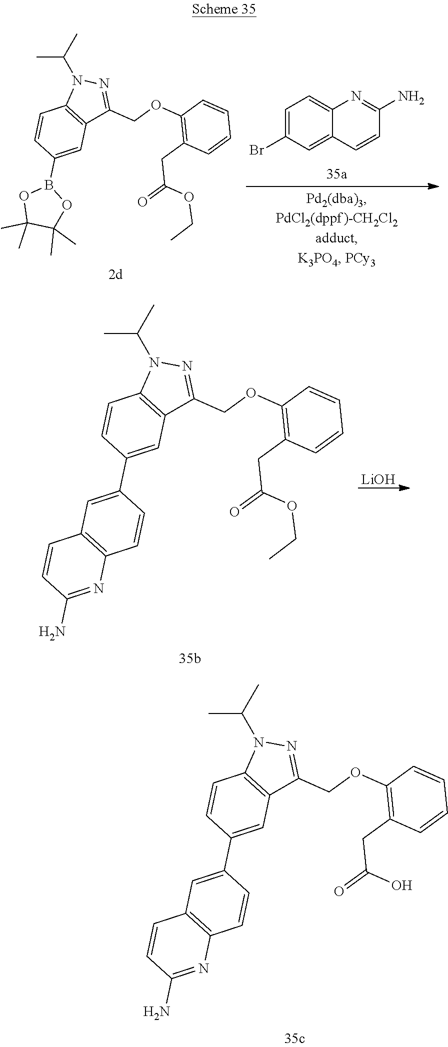

### Preparation of 2-(2-((5-(2-aminoquinolin-6-yl)-1-isopropyl-1H-indazol-3-yl)methoxy)phenyl)acetic acid (35c)

### Step-1: Preparation of ethyl 2-(2-((5-(2-aminoquinolin-6-yl)-1-isopropyl-1H-indazol-3-yl)methoxy)phenyl)acetate (35b)

Compound 35b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 6-bromoquinolin-2-amine (35a) (187 mg, 0.836 mmol; CAS #791626-58-9), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-R, ethyl 2-(2-((5-(2-aminoquinolin-6-yl)-1-isopropyl-1H-indazol-3-yl)methoxy)phenyl)acetate (35b) (150 mg, 72.5% yield) as a clear oil; 1H NMR (300 MHz, DMSO-d₆) δ 8.02 (d, J=1.3 Hz, 1H), 7.85 (dd, J=8.7, 2.2 Hz, 1H), 7.79 (d, J=1.2 Hz, 2H), 7.51 (d, J=8.7 Hz, 1H), 7.31-7.27 (m, 2H), 7.19 (d, J=7.3 Hz, 1H), 6.92 (dt, J=7.4, 4.4 Hz, 1H), 6.78 (d, J=9.0 Hz, 1H), 6.50 (s, 2H), 5.43 (s, 2H), 5.03 (p, J=6.6 Hz, 1H), 3.68-3.61 (m, 2H), 3.54 (s, 2H), 1.51 (d, J=6.6 Hz, 6H), 0.73 (t, J=7.1 Hz, 3H); MS (ES+): 495.20 (M+1).

### Step-2: Preparation of 2-(2-((5-(2-aminoquinolin-6-yl)-1-isopropyl-1H-indazol-3-yl)methoxy)phenyl)acetic acid (35c)

Compound 35c was prepared according to the procedure and method of purification reported in step-2 of scheme 1, from ethyl 2-(2-((5-(2-aminoquinolin-6-yl)-1-isopropyl-1H-indazol-3-yl)methoxy)phenyl)acetate (35b) (150 mg, 0.303 mmol) in THE/MeOH (4 mL, 1:1) using a solution of LiOH·H₂O (76 mg, 1.82 mmol) in water (1 mL) to afford after workup and purification using method-G, 2-(2-((5-(2-aminoquinolin-6-yl)-1-isopropyl-1H-indazol-3-yl)methoxy)phenyl)acetic acid (35c) (85 mg, 60.1% yield) HCl salt as a white solid; ¹H NMR (300 MHz, DMSO-d₆) δ 14.17 (s, 1H, D₂O exchangeable), 12.12 (s, 1H, D₂O exchangeable), 8.41 (d, J=9.3 Hz, 1H), 8.28 (d, J=2.0 Hz, 1H), 8.19-8.10 (m, 2H), 7.89-7.75 (m, 3H), 7.31-7.25 (m, 2H), 7.23-7.18 (m, 1H), 7.13 (d, J=9.3 Hz, 1H), 6.96-6.88 (m, 1H), 5.46 (s, 2H), 5.17-4.87 (m, 1H), 3.54 (s, 2H), 1.52 (d, J=6.6 Hz, 6H); MS (ES+): 467.20 (M+1); (ES−): 465.20 (M−1).

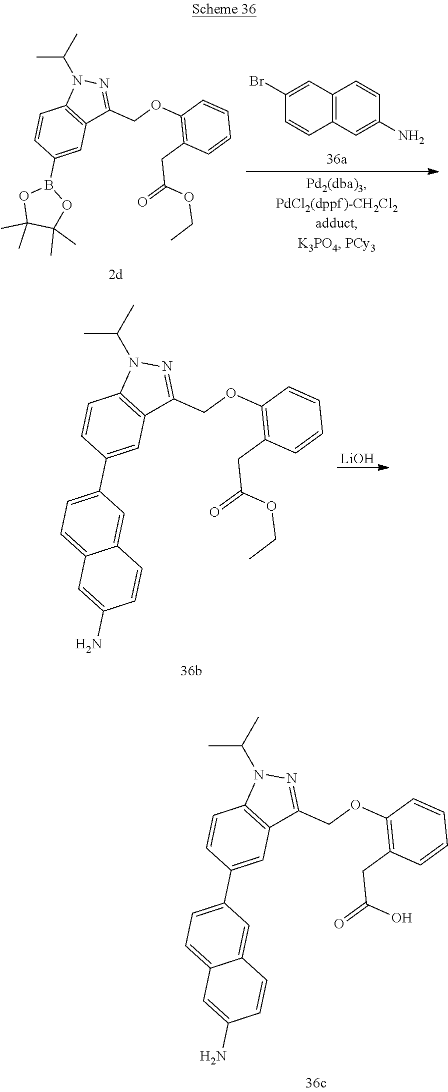

### Preparation of 2-(2-((5-(6-aminonaphthalen-2-yl)-1-isopropyl-1H-indazol-3-yl)methoxy)phenyl)acetic acid (36c)

### Step-1: Preparation of ethyl 2-(2-((5-(6-aminonaphthalen-2-yl)-1-isopropyl-1H-indazol-3-yl)methoxy)phenyl)acetate (36b)

Compound 36b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 6-bromonaphthalen-2-amine (36a) (186 mg, 0.836 mmol; CAS #7499-66-3), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(6-aminonaphthalen-2-yl)-1-isopropyl-1H-indazol-3-yl)methoxy)phenyl)acetate (36b) (160 mg, 78% yield) as a clear oil; ¹H NMR (300 MHz, DMSO-d₆) δ 8.01 (t, J=1.2 Hz, 1H), 7.95 (d, J=1.7 Hz, 1H), 7.79 (d, J=1.2 Hz, 2H), 7.69-7.64 (m, 2H), 7.58 (d, J=8.6 Hz, 1H), 7.30 (d, J=4.1 Hz, 2H), 7.19 (d, J=7.0 Hz, 2H), 6.96 (dd, J=6.5, 2.2 Hz, 1H), 6.83 (d, J=2.1 Hz, 1H), 5.44 (s, 2H), 5.03 (p, J=6.5 Hz, 1H), 3.69-3.62 (m, 2H), 3.54 (s, 2H), 1.52 (d, J=6.6 Hz, 6H), 0.73 (t, J=7.1 Hz, 3H); MS (ES+): 494.20 (M+); $16.20 (M+Na).

### Step-2: Preparation of 2-(2-((5-(6-aminonaphthalen-2-yl)-1-isopropyl-1H-indazol-3-yl)methoxy)phenyl)acetic acid (36c)

Compound 36c was prepared according to the procedure and method of purification reported in step-2 of scheme 1, from ethyl 2-(2-((5-(6-aminonaphthalen-2-yl)-1-isopropyl-1H-indazol-3-yl)methoxy)phenyl)acetate (36b) (160 mg, 0.324 mmol) in THE/MeOH (4 mL) using a solution of LiOH·H₂O (82 mg, 1.945 mmol) in water (1 mL) to afford after workup and purification using method-G, 2-(2-((5-(6-aminonaphthalen-2-yl)-1-isopropyl-1H-indazol-3-yl)methoxy)phenyl)acetic acid (36c) (75 mg, 0.161 mmol, 49.7% yield) HCl salt as a white solid; ¹H NMR (300 MHz, DMSO-d₆) δ 9.51 (s, 2H, D₂O exchangeable), 8.26 (s, 1H), 8.18 (s, 1H), 8.04 (d, J=8.8 Hz, 1H), 8.01-7.91 (m, 2H), 7.91-7.82 (m, 2H), 7.69 (s, 1H), 7.39 (dd, J=8.7, 2.2 Hz, 1H), 7.32-7.25 (m, 2H), 7.24-7.18 (m, 1H), 6.99-6.87 (m, 1H), 5.48 (s, 2H), 5.14-4.95 (m, 1H), 3.56 (s, 2H), 1.53 (d, J=6.6 Hz, 6H); MS (ES+): 466.20 (M+1); (ES−): 464.20 (M−1).

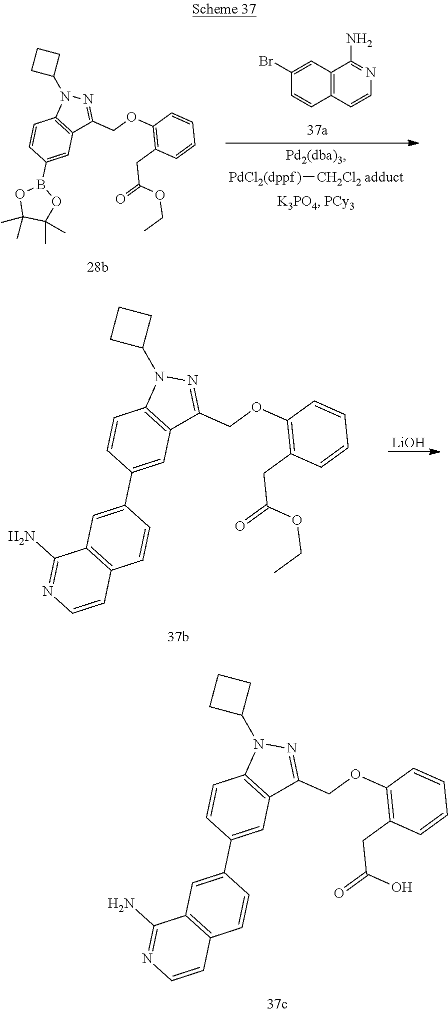

### Preparation of 2-(2-((5-(1-aminoisoquinolin-7-yl)-1-cyclobutyl-1H-indazol-3-yl)methoxy)phenyl)acetic acid (37c)

### Step-1: Preparation of ethyl 2-(2-((5-(1-aminoisoquinolin-7-yl)-1-cyclobutyl-1H-indazol-3-yl)methoxy)phenyl)acetate (37b)

Compound 37b was prepared according to the procedure and method of purification reported in step-5 of scheme 1, from ethyl 2-(2-((1-cyclobutyl-5-(4,4,5,5-tetramethyl-1,3,2-dioxaborolan-2-yl)-1H-indazol-3-yl)methoxy)phenyl)acetate (280 mg, 0.571 mmol) in dioxane/THF (4 mL, 1:1), using 7-bromoisoquinolin-1-amine (37a) (255 mg, 1.142 mmol; CAS #215453-53-5), K₃PO₄ (2M aqueous solution, 1.142 mL, 2.284 mmol), PCy₃ (32.0 mg, 0.114 mmol), Pd₂(dba)₃ (52.3 mg, 0.057 mmol) and PdCl₂(dppf)-CH₂Cl₂ adduct (46.6 mg, 0.057 mmol) to afford after workup and purification using method-E, ethyl 2-(2-((5-(1-aminoisoquinolin-7-yl)-1-cyclobutyl-1H-indazol-3-yl)methoxy)phenyl)acetate (37b) (200 mg, 69.1% yield) as a clear oil; 1H NMR (300 MHz, DMSO-d₆) δ 8.58-8.44 (m, 1H), 8.16 (d, J=1.6 Hz, 1H), 8.03 (dd, J=8.5, 1.6 Hz, 1H), 7.95 (dd, J=8.8, 1.6 Hz, 1H), 7.83 (d, J=8.5 Hz, 1H), 7.78 (dd, J=7.2, 4.5 Hz, 2H), 7.31 (dd, J=4.7, 1.6 Hz, 2H), 7.19 (d, J=7.3 Hz, 1H), 6.95-6.88 (m, 4H), 5.47 (s, 2H), 5.35 (p, J=8.4 Hz, 1H), 3.67-3.61 (m, 2H), 3.53 (s, 2H), 2.79-2.58 (m, 2H), 1.98-1.84 (m, 2H), 1.24-1.17 (m, 2H), 0.72 (t, J=7.1 Hz, 3H); MS (ES+): 507.20 (M+1); (ES−): 505.15 (M−1).

### Step-2: Preparation of 2-(2-((5-(1-aminoisoquinolin-7-yl)-1-cyclobutyl-1H-indazol-3-yl)methoxy)phenyl)acetic acid (37c)

Compound 37c was prepared according to the procedure and method of purification reported in step-2 of scheme 1, from ethyl 2-(2-((5-(1-aminoisoquinolin-7-yl)-1-cyclobutyl-1H-indazol-3-yl)methoxy)phenyl)acetate (37b) (200 mg, 0.395 mmol) in THE/MeOH (4 mL) using a solution of LiOH·H₂O (99 mg, 2.369 mmol) in water (1 mL) to afford after workup and purification using method-G, 2-(2-((5-(1-aminoisoquinolin-7-yl)-1-cyclobutyl-1H-indazol-3-yl)methoxy)phenyl)acetic acid (37c) (95 mg, 50.3% yield) HCl salt as a white solid; 1H NMR (300 MHz, DMSO-d₆) δ 9.17 (s, 2H, D₂O exchangeable), 8.96 (s, 1H), 8.41 (d, J=8.6, 1.6 Hz, 1H), 8.31 (s, 1H), 8.08-7.94 (m, 2H), 7.89 (d, J=8.9 Hz, 1H), 7.69 (d, J=6.9 Hz, 1H), 7.34-7.24 (m, 3H), 7.24-7.15 (m, 1H), 6.98-6.86 (m, 1H), 5.50 (s, 2H), 5.43-5.28 (m, 1H), 3.53 (s, 2H), 2.76-2.58 (m, 2H), 2.57-2.51 (m, 2H), 1.98-1.79 (m, 2H); MS (ES+): 479.20 (M+1); (ES−): 477.20 (M−1); Analysis calculated for C₂₉H₂₆N₄O₃·HCl·2H₂O: C, 63.21; H, 5.67; Cl, 6.43; N, 10.17; Found: C, 63.23; H, 5.69; Cl, 6.86; N, 10.00.

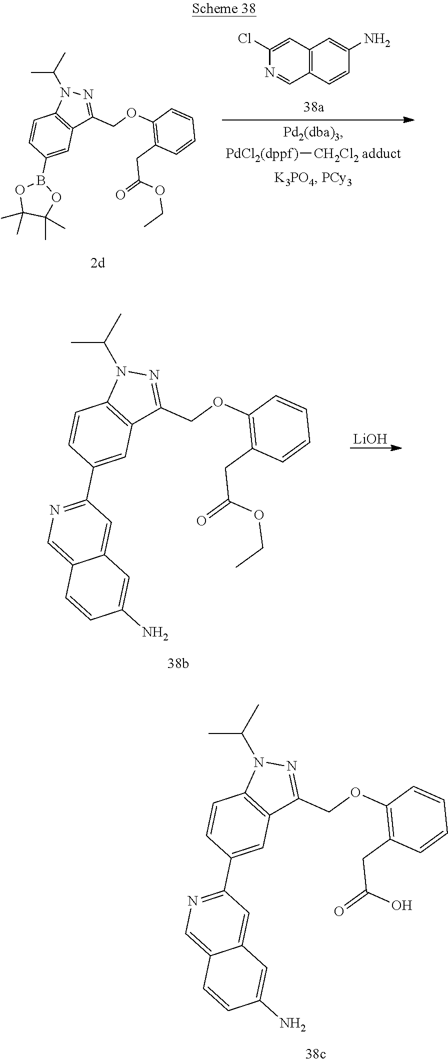

### Preparation of 2-(2-((5-(6-aminoisoquinolin-3-yl)-1-isopropyl-1H-indazol-3-yl)methoxy)phenyl)acetic acid (38c)

### Step-1: Preparation of ethyl 2-(2-((5-(6-aminoisoquinolin-3-yl)-1-isopropyl-1H-indazol-3-yl)methoxy)phenyl)acetate (38b)

Compound 38b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 3-chloroisoquinolin-6-amine (38a) (149 mg, 0.836 mmol; CAS #1374652-51-3), K₃PO₄ (2M aqueous solution, 0.836 mL, 1.672 mmol), PCy₃ (23.45 mg, 0.084 mmol). Pd₂(dba)₃ (38.3 mg, 0.042 mmol) and PdCl₂(dppf)-CH₂Cl₂ adduct (34.1 mg, 0.042 mmol) to afford after workup and purification using method-AC, ethyl 2-(2-((5-(6-aminoisoquinolin-3-yl)-1-isopropyl-1H-indazol-3-yl)methoxy)phenyl)acetate (38b) (80 mg, 38.7% yield) as a clear oil; ¹H NMR (300 MHz, DMSO-d₆) & 8.93 (s, 1H), 8.50 (d, J=1.6 Hz, 1H), 8.24 (dd, J=9.0, 1.6 Hz, 1H), 7.96 (s, 1H), 7.76 (t, J=9.5 Hz, 2H), 7.36-7.24 (m, 2H), 7.20 (d, J=7.3 Hz, 1H), 6.99 (d, J=2.1 Hz, JH), 6.96 (d, J=2.0 Hz, 1H), 6.79 (d, J=2.1 Hz, 1H), 6.00 (s, 2H), 5.45 (s, 2H), 5.04 (p, J=6.6 Hz, 1H), 3.73 (q, J=7.1 Hz, 2H), 3.55 (s, 2H), 1.52 (d, J=6.6 Hz, 6H), 0.76 (t, J=7.1 Hz, 3H); MS (ES+): 495.20 (M+1).

### Step-2: Preparation of 2-(2-((5-(6-aminoisoquinolin-3-yl)-1-isopropyl-1H-indazol-3-yl)methoxy)phenyl)acetic acid (38c)

Compound 38c was prepared according to the procedure and method of purification reported in step-2 of scheme 1, from ethyl 2-(2-((5-(6-aminoisoquinolin-3-yl)-1-isopropyl-1H-indazol-3-yl)methoxy)phenyl)acetate ((38b) (80 mg, 0.162 mmol) in THE/MeOH (4 mL, 1:1) using a solution of LiOH·H₂O (40.7 mg, 0.971 mmol) in water (I mL) to afford after workup and purification using method-G, 2-(2-((5-(6-aminoisoquinolin-3-yl)-1-isopropyl-1H-indazol-3-yl)methoxy)phenyl)acetic acid (38c) (20 mg, 26.5% yield) HCl salt as a yellow solid; ¹H NMR (300 MHz, DMSO-d₆) δ 9.17 (s, 1H), 8.44 (s, 1H), 8.19 (s, 1H), 8.13 (d, J=9.0 Hz, 1H), 8.07-7.93 (m, 2H), 7.37 (s, 2H, D₂O exchangeable), 7.31-7.18 (m, 4H), 6.99-6.88 (m, 2H), 5.48 (s, 2H), 5.18-4.99 (m, 1H), 3.54 (s, 2H), 1.54 (d, J=6.5 Hz, 6H); MS (ES+): 467.20 (M+1); (ES−): 465.20 (M−1).

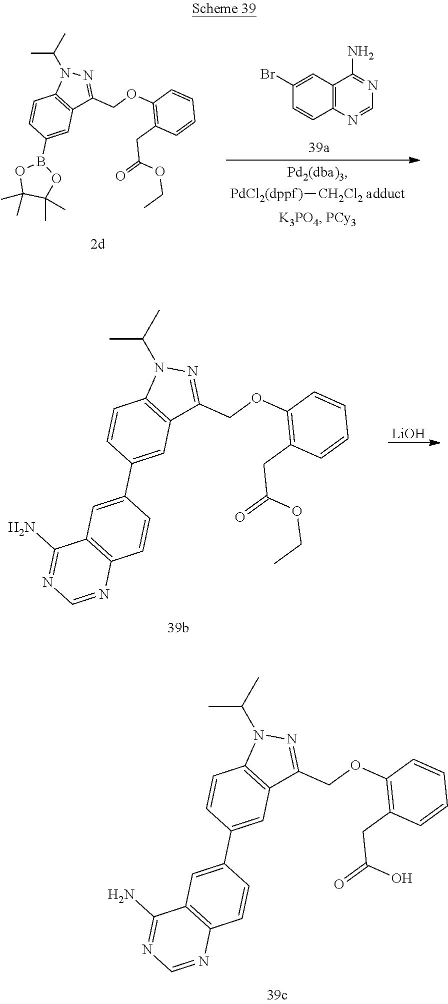

### Preparation of 2-(2-((5-(4-aminoquinazolin-6-yl)-1-isopropyl-1H-indazol-3-yl)methoxy)phenyl)acetic acid (39c)

### Step-1: Preparation of ethyl 2-(2-((5-(4-aminoquinazolin-6-yl)-1-isopropyl-1H-indazol-3-yl)methoxy)phenyl)acetate (39b)

Compound 39b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 6-bromoquinazolin-4-amine (39a) (187 mg, 0.836 mmol; CAS #21419-48-7), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(4-aminoquinazolin-6-yl)-1-isopropyl-1H-indazol-3-yl)methoxy)phenyl)acetate (39b) (150 mg, 72.4% yield) as a clear oil; ¹H NMR (300 MHz, DMSO-d₆) δ 8.57 (d, J=1.9 Hz, 1H), 8.38 (s, 1H), 8.17 (d, J=10.6 Hz, 2H), 7.92-7.86 (m, 2H), 7.73 (d, J=8.7 Hz, 1H), 7.30 (d, J=4.2 Hz, 2H), 7.19 (d, J=7.3 Hz, 1H), 6.93 (dq, J=8.0, 4.2 Hz, 1H), 5.44 (s, 2H), 5.06 (p, J=6.6 Hz, 1H), 3.65-3.60 (m, 2H), 3.53 (s, 2H), 1.52 (d, J=6.5 Hz, 6H), 0.71 (t, J=7.1 Hz, 3H); MS (ES+): 496.25 (M+1); (ES−): 494.20 (M−1).

### Step-2: Preparation of 2-(2-(5-(4-aminoquinazolin-6-yl)-1-isopropyl-1H-indazol-3-yl)methoxy)phenyl)acetic acid (39c)

Compound 39c was prepared according to the procedure and method of purification reported in step-2 of scheme 1, from ethyl 2-(2-((5-(4-aminoquinazolin-6-yl)-1-isopropyl-1H-indazol-3-yl)methoxy)phenyl)acetate (39b) (150 mg, 0.303 mmol) in THE/MeOH (4 mL, 1:1) using a solution of LiOH·H₂O (76 mg, 1.816 mmol) in water (1 mL) to afford after workup and purification using method-G, 2-(2-((5-(4-aminoquinazolin-6-yl)-1-isopropyl-1H-indazol-3-yl)methoxy)phenyl)acetic acid (39c) (95 mg, 0.203 mmol, 67.1% yield) HCl salt as a white solid; ¹H NMR (300 MHz, DMSO-d₆) δ 14.84 (s, 1H, D₂O) exchangeable), 12.13 (s, 1H, D₂O exchangeable), 9.93 (s, 1H, D₂O exchangeable), 9.76 (s, 1H, D₂O exchangeable), 8.89 (d, J=1.9 Hz, 1H), 8.82 (s, 1H), 8.48 (dd, J=8.8, 1.8 Hz, 1H), 8.27 (s, 1H), 8.02-7.86 (m, 3H), 7.33-7.24 (m, 2H), 7.24-7.15 (m, 1H), 6.99-6.86 (m, 1H), 5.47 (s, 2H), 5.15-4.09 (m, 1H), 3.54 (s, 2H), 1.53 (d, J=6.6 Hz, 6H); MS (ES+): 468.20 (M+1); (ES−): 466.15 (M−1).

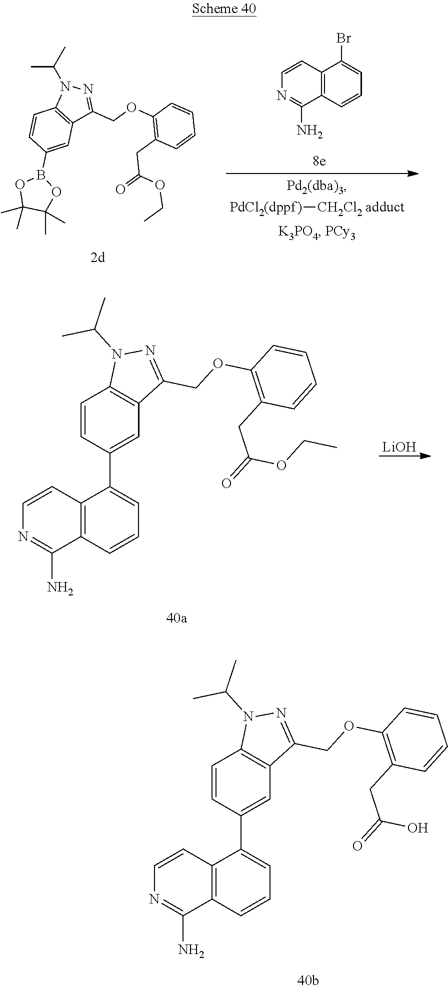

### Preparation of 2-(2-((5-(1-aminoisoquinolin-5-yl)-1-isopropyl-1H-indazol-3-yl)methoxy)phenyl)acetic acid (40b)

### Step-1: Preparation of ethyl 2-(2-((5-(1-aminoisoquinolin-5-yl)-1-isopropyl-1H-indazol-3-yl)methoxy)phenyl)acetate (40a)

Compound 40a was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 5-bromoisoquinolin-1-amine (5e) (187 mg, 0.836 mmol), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(1-aminoisoquinolin-5-yl)-1-isopropyl-1H-indazol-3-yl)methoxy)phenyl)acetate (40a) (150 mg, 72.5% yield) as a clear oil; ¹H NMR (300 MHz, DMSO-d₆) δ 8.21 (d, J=8.1 Hz, 1H), 7.83 (d, J=8.7 Hz, 1H), 7.80-7.73 (m, 2H), 7.62-7.53 (m, 2H), 7.53-7.44 (m, 2H), 7.26 (d, J=3.9 Hz, 2H), 7.16 (d, J=7.4 Hz, 1H), 6.92-6.85 (m, 2H), 6.83 (d, J=6.1 Hz, 1H), 5.42 (s, 2H), 5.07 (p, J=6.6 Hz, 1H), 3.58-3.51 (m, 2H), 3.50 (d, J=2.2 Hz, 2H), 1.54 (d, J=6.6 Hz, 6H), 0.69 (t, J=7.1 Hz, 3H); MS (ES+): 495.20 (M+1); (ES−): 493.05 (M−1).

### Step-2: Preparation of 2-(2-((5-(1-aminoisoquinolin-5-yl)-1-isopropyl-1H-indazol-3-yl)methoxy)phenyl)acetic acid (40b)

Compound 40b was prepared according to the procedure and method of purification reported in step-2 of scheme 1, from ethyl 2-(2-((5-(1-aminoisoquinolin-5-yl)-1-isopropyl-1H-indazol-3-yl)methoxy)phenyl)acetate (40a) (150 mg, 0.303 mmol) in THF/MeOH (4 mL, 1:1) using a solution of LiOH·H₂O (76 mg, 1.820 mmol) in water (1 mL) to afford after workup and purification using method-G, 2-(2-((5-(1-aminoisoquinolin-5-yl)-1-isopropyl-1H-indazol-3-yl)methoxy)phenyl)acetic acid (40b) (75 mg, 53.0% yield) HCl salt as a white solid; 3H NMR (300 MHz, DMSO-d₆) δ 13.33 (s, 1H, D₂O exchangeable), 12.03 (s, 1H, D₂O exchangeable), 9.21 (s, 2H, D₂O exchangeable), 8.64 (d, J=8.3 Hz, 1H), 7.97-7.89 (m, 1H), 7.87 (d, J=1.3 Hz, 2H), 7.83 (d, J=7.7 Hz, 1H), 7.63 (d, J=7.2 Hz, 1H), 7.46 (dd, J=8.6, 1.7 Hz, 1H), 7.30-7.20 (m, 2H), 7.20-7.12 (m, 1H), 6.99 (d, J=7.2 Hz, 1H), 6.94-6.84 (m, 1H), 5.45 (s, 2H), 5.17-4.97 (m, 1H), 3.48 (s, 2H), 1.54 (d, J=6.6 Hz, 6H); MS (ES+): 467.20 (M+1); (ES−): 465.10 (M−1).

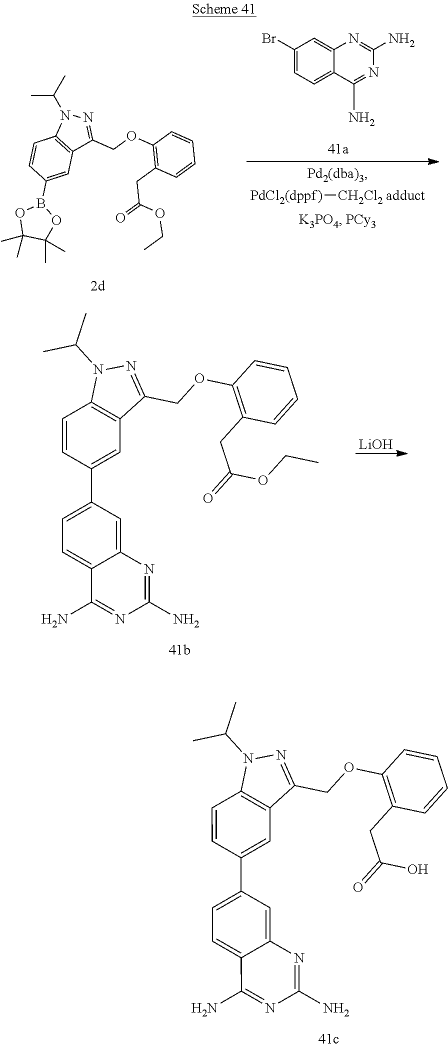

### Preparation of 2-(2-((5-(2,4-diaminoquinazolin-7-yl)-1-isopropyl-1H-indazol-3-yl)methoxy)phenyl)acetic acid (41c)

### Step-1: Preparation of ethyl 2-(2-((5-(2,4-diaminoquinazolin-7-yl)-1-isopropyl-1H-indazol-3-yl)methoxy)phenyl)acetate (41b)

Compound 41b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 7-bromoquinazoline-2,4-diamine (41a) (200 mg, 0.836 mmol; CAS #137553-43-6), K₃PO₄ (2M aqueous solution, 0.836 mL, 1.672 mmol), PCy₃ (23.45 mg, 0.084 mmol). Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(2,4-diaminoquinazolin-7-yl)-1-isopropyl-1H-indazol-3-yl)methoxy)phenyl)acetate (41b) as a clear oil; 1H NMR (300 MHz, DMSO-d₆) δ 8.09-8.01 (m, 2H), 7.82-7.76 (m, 2H), 7.47 (d, J=1.8 Hz, 1H), 7.38 (d, J=8.0 Hz, 2H), 7.29 (d, J=4.1 Hz, 1H), 7.19 (d, J=7.3 Hz, 1H), 6.92 (dt, J=8.0, 4.2 Hz, 1H), 6.18 (s, 2H), 6.02 (s, 2H), 5.44 (s, 2H), 5.11-4.95 (m, 1H), 3.66 (q, J=7.1 Hz, 2H), 3.52 (s, 2H), 1.52 (d, J=6.6 Hz, 6H), 0.74 (t, J=7.1 Hz, 3H); MS (ES+): 511.25 (M+1); (ES−): 509.20 (M−1).

### Step-2: Preparation of 2-(2-((5-(2,4-diaminoquinazolin-7-yl)-1-isopropyl-1H-indazol-3-yl)methoxy)phenyl)acetic acid (41c)

Compound 41c was prepared according to the procedure and method of purification reported in step-2 of scheme 1, from ethyl 2-(2-((5-(2,4-diaminoquinazolin-7-yl)-1-isopropyl-1H-indazol-3-yl)methoxy)phenyl)acetate (41b) (150 mg, 0.294 mmol) in THF/MeOH (4 mL, 1:1) using a solution of LiOH·H₂O (74.0 mg, 1.763 mmol) in water (I mL) to afford after workup and purification using method-G, 2-(2-((5-(2,4-diaminoquinazolin-7-yl)-1-isopropyl-1H-indazol-3-yl)methoxy)phenyl)acetic acid (41c) (80 mg, 56.4% yield) HCl salt as a white solid; ¹H NMR (300 MHz, DMSO-d₆) δ 9.04 (s, 1H, D₂O exchangeable), 8.80 (s, 1H, D₂O exchangeable), 8.33 (d, J=8.6 Hz, 1H), 8.20 (d, J=1.6 Hz, 1H), 7.89 (d, J=8.9 Hz, 1H), 7.80 (dd, J=8.7, 1.7 Hz, 2H), 7.72 (d, J=1.7 Hz, 1H), 7.32-7.23 (m, 2H), 7.20 (d, J=7.4 Hz, 1H), 6.98-6.84 (m, 1H), 5.47 (s, 2H), 5.13-4.92 (m, 1H), 3.53 (s, 2H), 1.52 (d, J=6.5 Hz, 6H); MS (ES+): 483.20 (M+1); (ES−): 481.20 (M−1).

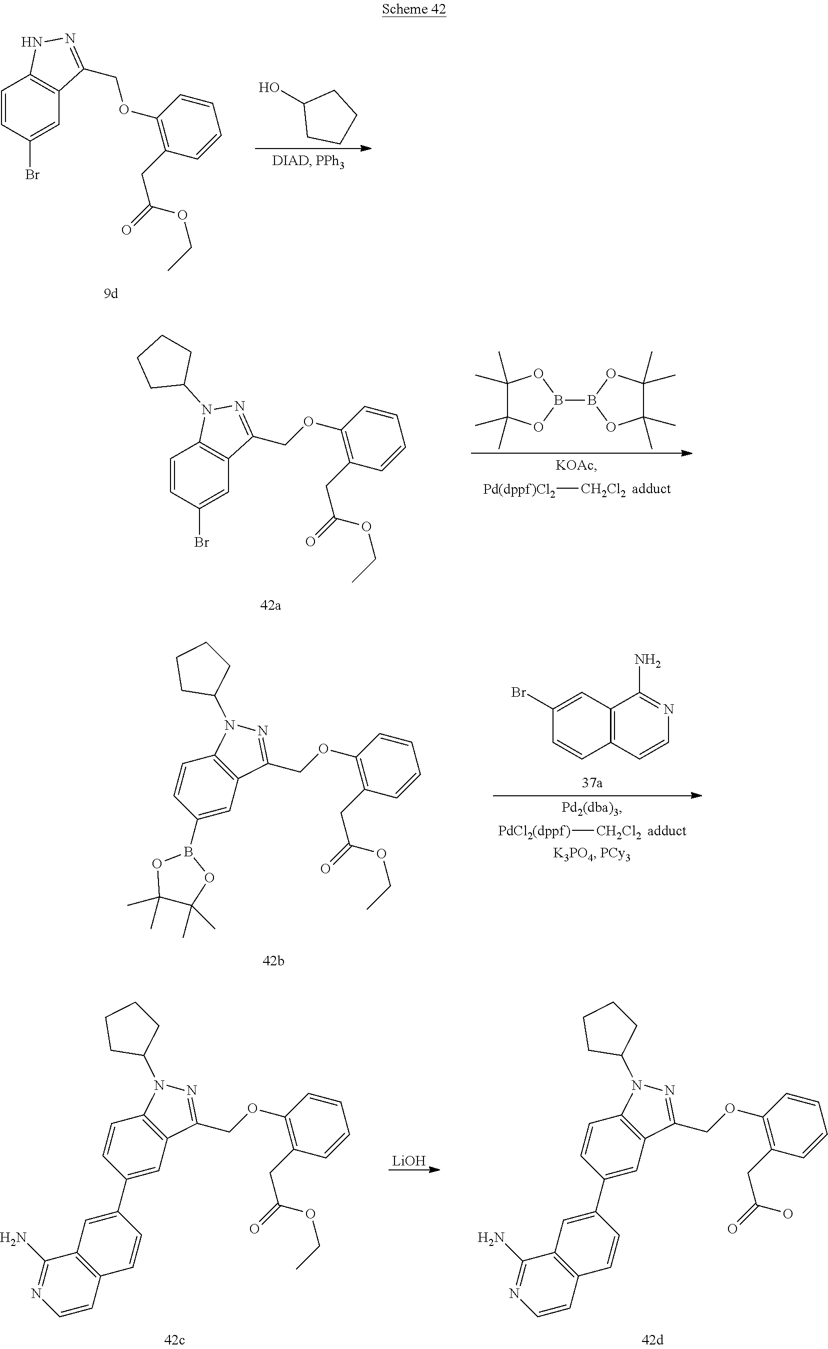

### Preparation of 2-(2-((5-(1-aminoisoquinolin-7-yl)-1-cyclopentyl-1H-indazol-3-yl)methoxy)phenyl)acetic acid (42d)

### Step-1: Preparation of ethyl 2-(2-((5-bromo-1-cyclopentyl-1H-indazol-3-yl)methoxy)phenyl)acetate (42a)

To a solution of triphenylphosphine (606 mg, 2.312 mmol), cyclopentanol (199 mg, 2.312 mmol) and ethyl 2-(2-((5-bromo-1H-indazol-3-yl)methoxy)phenyl)acetate (9d) (500 mg, 1.285 mmol) in THE was added drop wise at 0° C. diisopropyl azodicarboxylate (DIAD) (0.450 mL, 2.312 mmol) and allowed to warm to room temperature overnight. Additional cyclopentanol (3.6 eq.), DIAD (1.8 eq.) and TPP (1.8 eq.) were added to drive reaction to completion. The reaction was concentrated in vacuum and purified using method-X, to afford ethyl 2-(2-((5-bromo-1-cyclopentyl-1H-indazol-3-yl)methoxy)phenyl)acetate (42a) (200 mg, 34.0% yield) as a clear oil; JH NMR (300 MHz, DMSO-d₆) δ 7.94 (d, J=1.8 Hz, 1H), 7.72 (d, J=9.0 Hz, 1H), 7.52 (dd, J=8.9, 1.9 Hz, 1H), 7.31-7.15 (m, 3H), 6.97-6.84 (m, 1H), 5.36 (s, 2H), 5.24-5.08 (m, 1H), 3.91 (q, J=7.1 Hz, 2H), 3.53 (s, 2H), 2.14 (dt, J=13.1, 6.5 Hz, 2H), 2.04-1.82 (m, 4H), 1.76-1.61 (m, 2H), 0.94 (t, J=7.1 Hz, 3H); MS (ES+): 457.10 & 459.10 (M+1); 479.10 & 481.10 (M+Na).

### Step-2: Preparation of ethyl 2-(2-((1-cyclopentyl-5-(4,4,5,5-tetramethyl-1,3,2-dioxaborolan-2-yl)-1H-indazol-3-yl)methoxy)phenyl)acetate (42b)

Compound 42b was prepared according to the procedure and method of purification reported in step-4 of scheme 1, from ethyl 2-(2-((5-bromo-1-cyclopentyl-1H-indazol-3-yl)methoxy)phenyl)acetate (42a) (650 mg, 1.421 mmol) in anhydrous dioxane (20 mL), using BISPIN (722 mg, 2.84 mmol), KOAc (349 mg, 3.55 mmol), PdCl₂(dppf)-CH₂Cl₂ adduct (69.6 mg, 0.085 mmol) and heating for 15 h at 100° ° C. The reaction mixture was then diluted with EtOAc (100 mL) and washed with water (100 mL). This gave after workup and purification using method-C, ethyl 2-(2-((1-cyclopentyl-5-(4,4,5,5-tetramethyl-1,3,2-dioxaborolan-2-yl)-1H-indazol-3-yl)methoxy)phenyl)acetate (42b) (580 mg, 81% yield) as a clear oil; ¹H NMR (300 MHz, DMSO-d₆) δ 8.15 (d, J=1.0 Hz, 1H), 7.74-7.60 (m, 2H), 7.33-7.23 (m, 2H), 7.19 (d, J=7.3 Hz, 1H), 6.97-6.85 (m, 1H), 5.39 (s, 2H), 5.17 (p, J=6.9 Hz, 1H), 3.86 (q. J=7.1 Hz, 2H), 3.50 (s, 2H), 2.20-2.07 (m, 2H), 1.99 (dt, J=13.7, 6.8 Hz, 2H), 1.85 (dd, J=14.0, 7.8 Hz, 2H), 1.78-1.63 (m, 2H), 1.29 (s, 12H), 0.88 (t, J=7.1 Hz, 3H); MS (ES+): 505.30 (M+1).

### Step-3: Preparation of ethyl 2-(2-((5-(1-aminoisoquinolin-7-yl)-1-cyclopentyl-1H-indazol-3-yl)methoxy)phenyl)acetate (42c)

Compound 42c was prepared according to the procedure and method of purification reported in step-5 of scheme 1, from ethyl 2-(2-((1-cyclopentyl-5-(4,4,5,5-tetramethyl-1,3,2-dioxaborolan-2-yl)-1H-indazol-3-yl)methoxy)phenyl)acetate (42b) (250 mg, 0.496 mmol) in dioxane/THF (4 mL, 1:1), using 7-bromoisoquinolin-1-amine (37a) (166 mg, 0.743 mmol), K₃PO₄ (2M aqueous solution, 0.991 mL, 1.982 mmol), PCy₃ (27.8 mg, 0.099 mmol), Pd₂(dba)₃ (45.4 mg, 0.050 mmol), PdCl₂(dppf)-CH₂Cl₂ adduct (40.5 mg, 0.050 mmol) and beating at 100° ° C. for 2 h on oil bath. This gave after workup and purification using method-E, ethyl 2-(2-((5-(1-aminoisoquinolin-7-yl)-1-cyclopentyl-1H-indazol-3-yl)methoxy)phenyl)acetate (42c) as a clear oil; ¹H NMR (300 MHz, DMSO-d₆) δ 8.53 (s, 1H), 8.16 (d, J=1.6 Hz, 1H), 8.03 (dd, J=8.5, 1.7 Hz, 1H), 7.95 (dd, J=8.9, 1.7 Hz, 1H), 7.84 (d, J=8.9 Hz, 1H), 7.80-7.74 (m, 2H), 7.31-7.27 (m, 2H), 7.22-7.15 (m, 1H), 6.96-6.87 (m, 4H), 5.44 (s, 2H), 5.23 (p, J=7.0 Hz, 1H), 3.63 (q, J=7.1 Hz, 2H), 3.53 (s, 2H), 2.17 (q, J=5.6 Hz, 2H), 2.04 (h, J=6.8 Hz, 2H), 1.93-1.86 (m, 2H), 1.75-1.70 (m, 2H), 0.72 (t, J=7.1 Hz, 3H); MS (ES+): 521.35 (M+1).

### Step-4: Preparation of 2-(2-((5-(1-aminoisoquinolin-7-yl)-1-cyclopentyl-1H-indazol-3-yl)methoxy)phenyl)acetic acid (42d)

Compound 42d was prepared according to the procedure and method of purification reported in step-2 of scheme 1, from ethyl 2-(2-((5-(1-aminoisoquinolin-7-yl)-1-cyclopentyl-1H-indazol-3-yl)methoxy)phenyl)acetate (42c) (150 mg, 0.288 mmol) in THE/MeOH (4 mL, 1:1) using a solution of LiOH·H₂O (72.5 mg, 1.729 mmol) in water (1 mL) to afford after workup and purification using method-G, 2-(2-((5-(1-aminoisoquinolin-7-yl)-1-cyclopentyl-1H-indazol-3-yl)methoxy)phenyl)acetic acid (42d) (105 mg, 74.0% yield) HCl salt as a white solid; ¹H NMR (300 MHz, DMSO-d₆) δ 9.12 (s, 2H, D₂O exchangeable), 8.95 (s, 1H), 8.40 (dd, J=8.6, 1.6 Hz, 1H), 8.30 (s, 1H), 8.06-7.95 (m, 2H), 7.90 (d, J=8.9 Hz, 1H), 7.69 (d, J=6.9 Hz, 1H), 7.33-7.22 (m, 3H), 7.22-7.15 (m, 1H), 6.92 (td, J=7.0, 1.9 Hz, 1H), 5.48 (s, 2H), 5.32-5.17 (m, 1H), 3.53 (s, 2H), 2.25-2.12 (m, 2H), 2.12-1.98 (m, 2H), 1.96-1.84 (m, 2H), 1.79-1.67 (m, 2H); MS (ES+): 493.20 (M+1); (ES−): 491.20 (M−1).

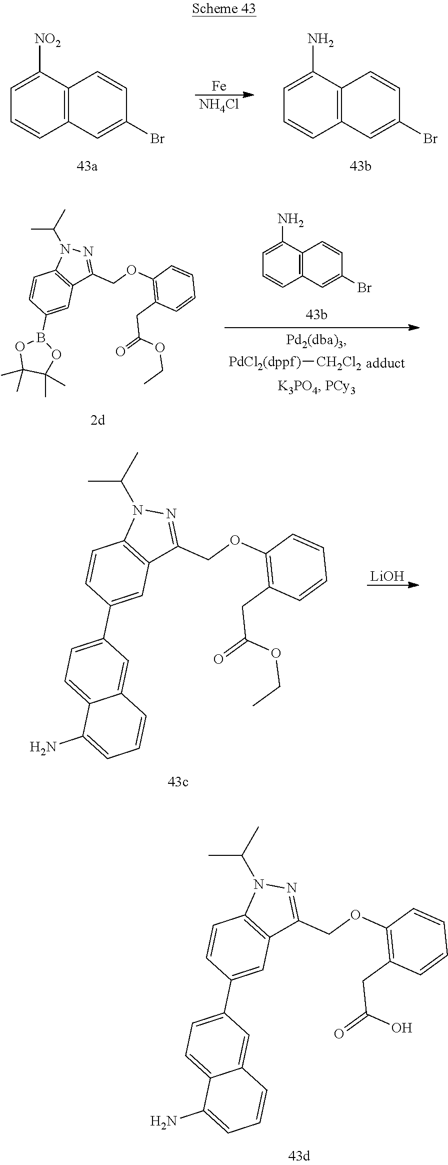

### Preparation of 2-(2-((5-(5-aminonaphthalen-2-yl)-1-isopropyl-1H-indazol-3-yl)methoxy)phenyl)acetic acid (43d)

### Step-1: Preparation of 6-bromonaphthalen-1-amine (43b)

To a solution of 6-bromo-1-nitronaphthalene (43a) (400 mg, 1.587 mmol) in ethanol (5 mL) was added an aqueous solution of ammonium chloride (1698 mg, 31.7 mmol) in water (5 mL), iron (886 mg, 15.87 mmol) and heated for 2 hours at 60° C. The mixture was then cooled to 22° C., diluted with EtOAc (30 mL) and stirred for 1 h at 22° C. The mixture was filtered through a pad of Celite and rinsed with EtOAc (3×20 mL). The combined filtrate was concentrated to a volume of ca. 50 mL diluted with water (50 mL) and EtOAc (200 mL) and stirred for 1 h. The reaction mixture was extracted with EtOAc (3×100 mL) and the combined organics were washed with water, brine, dried, filtered and concentrated in vacuum to give 6-bromonaphthalen-1-amine (43b) (280 mg, 79% yield) as a brown solid, which was used in the next step without further purification: 1H NMR (300 MHz, DMSO-d₆) δ 8.02 (d, J=9.0 Hz, 1H), 7.97 (d, J=2.1 Hz, 1H), 7.45 (dd, J=9.0, 2.1 Hz, 1H), 7.24 (t, J=7.8 Hz, 1H), 7.04 (d, J=8.1 Hz, 1H), 6.69 (dd, J=7.5, 1.1 Hz, 1H), 5.86 (s, 2H); MS (ES+): 222.00 & 224.00 (M+1).

### Step-2: Preparation of ethyl 2-(2-((5-(5-aminonaphthalen-2-yl)-1-isopropyl-1H-indazol-3-yl)methoxy)phenyl)acetate (43c)

Compound 43c was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL), using 6-bromonaphthalen-1-amine (43b) (139 mg, 0.627 mmol), K₃PO₄ (2M aqueous solution, 0.836 mL, 1.672 mmol), PCy₃ (23.45 mg, 0.084 mmol), Pd₂(dba)₃ (38.3 mg, 0.042 mmol), PdCl₂(dppf)-CH₂Cl₂ adduct (34.1 mg, 0.042 mmol) to afford after workup and purification using method-AC, ethyl 2-(2-((5-(5-aminonaphthalen-2-yl)-1-isopropyl-1H-indazol-3-yl)methoxy)phenyl)acetate (43c) (150 mg, 72.7% yield) as a clear oil; 1H NMR (300 MHz, DMSO-d₆) δ 8.19-8.08 (m, 2H), 8.04 (d, J=1.9 Hz, 1H), 7.91-7.78 (m, 2H), 7.74 (dd, J=8.8, 1.9 Hz, 1H), 7.34-7.26 (m, 2H), 7.26-7.11 (m, 3H), 6.97-6.87 (m, 1H), 6.65 (dd, J=7.2, 1.3 Hz, 1H), 5.76 (s, 2H), 5.46 (s, 2H), 5.04 (h, J=6.6 Hz, 1H), 3.66 (q, J=7.1 Hz, 2H), 3.55 (s, 2H), 1.52 (d, J=6.6 Hz, 6H), 0.73 (t, J=7.1 Hz, 3H); MS (ES+): 494.20 (M+1); (ES−): 492.20 (M−1).

### Step-3: Preparation of 2-(2-((5-(5-aminonaphthalen-2-yl)-1-isopropyl-1H-indazol-3-yl)methoxy)phenyl)acetic acid (43d)

Compound 43d was prepared according to the procedure and method of purification reported in step-2 of scheme 1, from ethyl 2-(2-((5-(5-aminonaphthalen-2-yl)-1-isopropyl-1H-indazol-3-yl)methoxy)phenyl)acetate (43c) (150 mg, 0.304 mmol) in THE/MeOH (4 mL) using a solution of LiOH·H₂O (77 mg, 1.823 mmol) in water (1 mL) to afford after workup and purification using method-G, 2-(2-((5-(5-aminonaphthalen-2-yl)-1-isopropyl-1H-indazol-3-yl)methoxy)phenyl)acetic acid (43d) (65 mg, 45.9% yield) HCl salt as an orange solid; 1H NMR (300 MHz, DMSO-d₆) δ 8.34 (d, J=1.8 Hz, 1H), 8.24 (s, 1H), 8.16 (d, J=8.9 Hz, 1H), 8.06 (dd, J=8.8, 1.9 Hz, 1H), 7.94-7.83 (m, 3H), 7.53 (t, J=7.7 Hz, 1H), 7.49-7.41 (m, 1H), 7.34-7.25 (m, 2H), 7.25-7.17 (m, 1H), 6.92 (td, J=7.0, 2.0 Hz, 1H), 5.49 (s, 2H), 5.12-4.99 (m, 1H), 3.56 (s, 2H), 1.53 (d, J=6.6 Hz, 6H); MS (ES+): 466.20 (M+1); (ES−): 464.10 (M−1); Analysis calculated for C₂₉H₂₇N₃O₃·HCl·H₂O: C, 66.98; H, 5.82; Cl, 6.82; N, 8.08; Found: C, 66.97; H, 5.73; Cl, 6.67; N, 8.12.

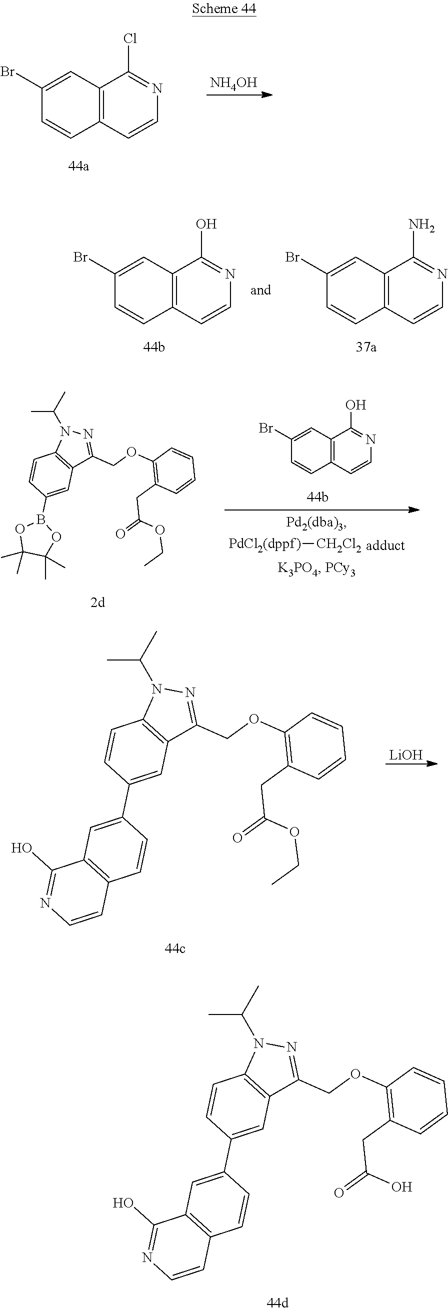

### Preparation of 2-(2-((5-(1-hydroxyisoquinolin-7-yl)-1-isopropyl-1H-indazol-3-yl)methoxy)phenyl)acetic acid (44d)

### Step-1: Preparation of 7-bromoisoquinolin-1-ol (44b)

To a solution of 7-bromo-1-chloroisoquinoline (44a) (5 g, 20.62 mmol; CAS #215453-51-3) in N-methyl-2-pyrrolidinone (40 mL) was added aqueous ammonium hydroxide (16.06 mL, 412 mmol) and heated at 150° ° C. for 3 days. The mixture was cooled to RT poured into water (200 mL) and extracted with EtOAc (500 mL×4). Combined organics were washed with brine (300 mL), dried, concentrated and the residue obtained was purified by using method-AD to give 7-bromoisoquinolin-1-ol (44b) (200 mg, 4.33% yield) as a white solid; ¹H NMR (300 MHz, DMSO-d₆) δ 11.45 (s, 1H), 8.25 (d, J=2.2 Hz, 1H), 7.84 (dd, J=8.5, 2.2 Hz, 1H), 7.64 (d, J=8.5 Hz, 1H), 7.27-7.16 (m, 1H), 6.57 (d, J=7.1 Hz, 1H); MS (ES+): 224.00 (M+1); and 7-bromoisoquinolin-1-amine (37a) (2.3 g, 50.0% yield) as a white solid; 1H NMR (300 MHz, DMSO-d₆) δ 8.48 (d, J=1.9 Hz, 1H), 7.83 (d, J=5.8 Hz, 1H), 7.75-7.71 (m, 1H), 7.66 (d, J=8.7 Hz, 1H), 6.92 (m, 1H), 6.90 (s, 2H); MS (ES+): 222.95 (M+1).

### Step-2: Preparation of ethyl 2-(2-(5-(1-hydroxyisoquinolin-7-yl)-1-isopropyl-1H-indazol-3-yl)methoxy)phenyl)acetate (44c)

Compound 44c was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 7-bromoisoquinolin-1-ol (44b) (141 mg, 0.627 mmol), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(1-hydroxyisoquinolin-7-yl)-1-isopropyl-1H-indazol-3-yl)methoxy)phenyl)acetate (44c) (130 mg, 62.7% yield) as a clear oil; 1H NMR (300 MHz, DMSO-d₆) δ 11.30 (d, J=5.8 Hz, 1H), 8.46 (d, J=2.0 Hz, 1H), 8.12-8.01 (m, 2H), 7.88-7.78 (m, 2H), 7.75 (d, J=8.3 Hz, 1H), 7.33-7.26 (m, 2H), 7.18 (dt, J=7.1, 2.8 Hz, 2H), 6.92 (dt, J=7.3, 4.2 Hz, 1H), 6.59 (d, J=7.0 Hz, 1H), 5.46 (s, 2H), 5.04 (h, J=6.6 Hz, 1H), 3.62 (q, J=7.1 Hz, 2H), 3.52 (s, 2H), 1.52 (d, J=6.5 Hz, 6H), 0.73 (t, J=7.1 Hz, 3H); MS (ES+): 496.20 (M+1); (ES−): 494.20 (M−1).

### Step-3: Preparation of 2-(2-((5-(1-hydroxyisoquinolin-7-yl)-1-isopropyl-1H-indazol-3-yl)methoxy)phenyl)acetic acid (44d)

Compound 44d was prepared according to the procedure and method of purification reported in step-2 of scheme 1, from ethyl 2-(2-((5-(1-hydroxyisoquinolin-7-yl)-1-isopropyl-1H-indazol-3-yl)methoxy)phenyl)acetate (44c) (130 mg, 0.262 mmol) in THE/MeOH (4 mL, 1:1) using a solution of LiOH·H₂O (66.0 mg, 1.574 mmol) in water (1 mL) to afford after workup and purification using method-G, 2-(2-((5-(1-hydroxyisoquinolin-7-yl)-1-isopropyl-1H-indazol-3-yl)methoxy)phenyl)acetic acid (44d) (55 mg, 44.8% yield) HCl salt as a white solid; 1H NMR (300 MHz, DMSO-d₆) δ 12.22 (s, 1H, DO exchangeable), 11.43 (d, J=5.7 Hz, 1H, D₂O exchangeable), 8.51 (d, J=2.0 Hz, 1H), 8.17-8.04 (m, 2H), 7.86-7.80 (m, 2H), 7.77 (d, J=8.4 Hz, 1H), 7.33-7.24 (m, 2H), 7.24-7.15 (m, 2H), 6.96-6.85 (m, 1H), 6.64 (d, J=7.0 Hz, 1H), 5.49 (s, 2H), 5.16-4.90 (m, 1H), 3.56 (s, 2H), 1.52 (d, J=6.5 Hz, 6H); MS (ES+): 468.20 (M+1); (ES−): 466.10 (M−1); Analysis calculated for C₂₈H₂₅N₃O₄·0.1HCl: C, 71.38; H, 5.37; Cl, 0.75; N, 8.92; Found: C, 71.35; H, 5.28; Cl, 1.00; N, 8.88.

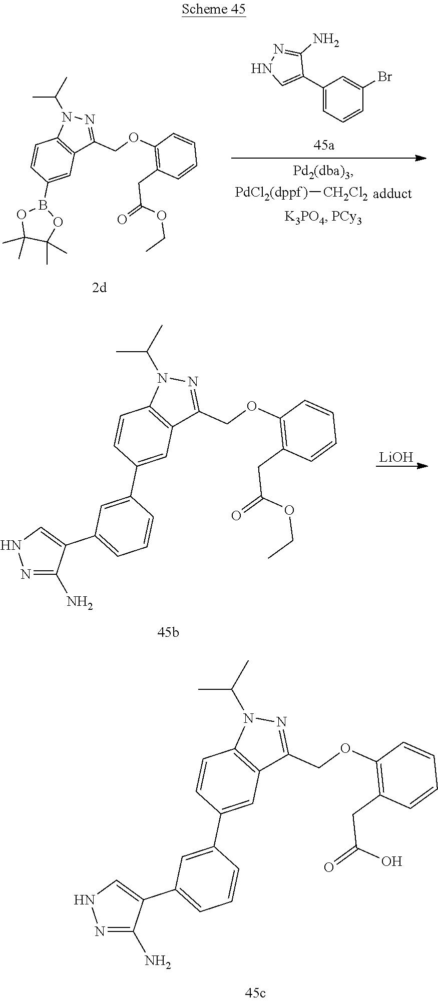

### Preparation of 2-(2-((5-(3-(3-amino-1H-pyrazol-4-yl)phenyl)-1-isopropyl-1H-indazol-3-yl)methoxy)phenyl)acetic acid (45c)

### Step-1: Preparation of ethyl 2-(2-((5-(3-(3-amino-1H-pyrazol-4-yl)phenyl)-1-isopropyl-1H-indazol-3-yl)methoxy)phenyl)acetate (45b)

Compound 45b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 4-(3-bromophenyl)-1H-pyrazol-3-amine (45a) (199 mg, 0.836 mmol; CAS #301373-51-3), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-U, ethyl 2-(2-((5-(3-(3-amino-1H-pyrazol-4-yl)phenyl)-1-isopropyl-1H-indazol-3-yl)methoxy)phenyl)acetate (45b) (100 mg, 46.9% yield) as a clear oil; ¹H NMR (300 MHZ, DMSO-d₆) δ 11.73 (s, 1H), 8.01 (t, J=1.3 Hz, 1H), 7.77 (d, J=10.4 Hz, 3H), 7.59-7.35 (m, 4H), 7.34-7.23 (m, 3H), 7.19 (d, J=7.3 Hz, 1H), 6.91 (dt, J=8.2, 4.2 Hz, 1H), 5.43 (s, 2H), 5.04 (p, J=6.7 Hz, 1H), 3.66 (q, J=7.1 Hz, 2H), 3.54 (s, 2H), 1.51 (d, J=6.6 Hz, 6H), 0.77 (t, J=7.1 Hz, 3H); MS (ES+): 510.25 (M+1); (ES−): 508.20 (M−1).

### Step-2: Preparation of Preparation of 2-(2-((5-(3-(3-amino-1H-pyrazol-4-yl)phenyl)-1-isopropyl-1H-indazol-3-yl)methoxy)phenyl)acetic acid (45c)

Compound 45c was prepared according to the procedure and method of purification reported in step-2 of scheme 1, from ethyl 2-(2-((5-(3-(3-amino-1H-pyrazol-4-yl)phenyl)-1-isopropyl-1H-indazol-3-yl)methoxy)phenyl)acetate (45b) (100 mg, 0.196 mmol) in THE/MeOH (4 mL, 1:1) using a solution of LiOH·H₂O (49.4 mg, 1.177 mmol) in water (1 mL) to afford after workup and purification using method-G, 2-(2-((5-(3-(3-amino-1H-pyrazol-4-yl)phenyl)-1-isopropyl-1H-indazol-3-yl)methoxy)phenyl)acetic acid (45c) (55 mg, 58.2% yield) HCl salt as a white solid; ¹H NMR (300 MHz, DMSO-d₆) δ 8.25 (s, 1H), 8.11 (s, 1H), 7.85 (s, 1H), 7.82 (s, 2H), 7.66-7.59 (m, 1H), 7.51 (d, J=4.9 Hz, 2H), 7.32-7.24 (m, 2H), 7.24-7.16 (m, 1H), 6.91 (td, J=7.0, 1.8 Hz, 1H), 5.46 (s, 2H), 5.12-4.97 (m, 1H), 3.52 (s, 2H), 1.52 (d, J=6.6 Hz, 6H); MS (ES+): 482.20 (M+1); (ES−): 480.20 (M−1); Analysis calculated for C₂₈H₂₇N₅O₃·HCl·1.25H₂O: C, 62.22; H, 5.69; Cl, 6.56; N, 12.96; Found: C, 62.20; H, 5.52; Cl, 6.49; N, 12.88.

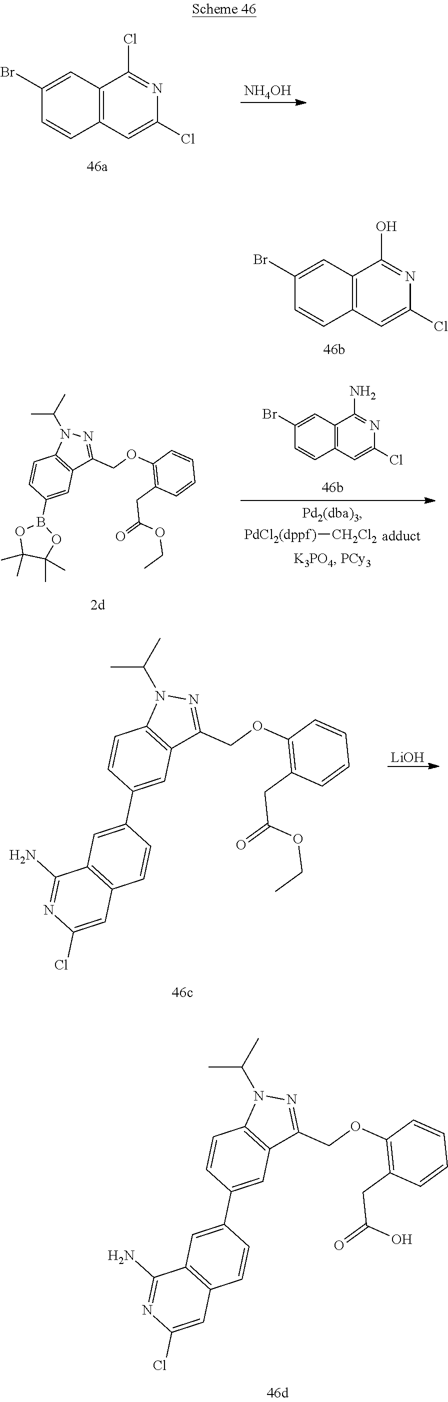

### Preparation of 2-(2-((5-(1-amino-3-chloroisoquinolin-7-yl)-1-isopropyl-1H-indazol-3-yl)methoxy)phenyl)acetic acid (46d)

### Step-1: Preparation of 7-bromo-3-chloroisoquinolin-1-amine (46b)

Compound 46b was prepared according to the procedure and method of purification reported in step-1 of scheme 44, from 7-bromo-1,3-dichloroisoquinoline (46a) (1 g, 3.61 mmol; CAS #924271-40-9) in N-Methyl-2-pyrrolidinone (20 mL) using aqueous NH₄OH (5.62 mL, 144 mmol) and heating at 150ºC for two days. This gave after workup and purification using method-O, 7-bromo-3-chloroisoquinolin-1-amine (46b) (820 mg, 88% yield) as a white solid; 1H NMR (300 MHz, DMSO-d₆) δ 8.49 (d, J=1.9 Hz, 1H), 7.77 (dd, J=8.7, 1.9 Hz, 1H), 7.64 (d, J=8.7 Hz, 1H), 7.45 (s, 2H), 7.00 (s, 1H); MS (ES+): 256.90 & 258.90 (M+1); (ES−): 254.90 (M−1).

### Step-2: Preparation of ethyl 2-(2-((5-(1-amino-3-chloroisoquinolin-7-yl)-1-isopropyl-1H-indazol-3-yl)methoxy)phenyl)acetate (46c)

Compound 46c was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (100 mg, 0.209 mmol) in dioxane/THF (4 mL, 1:1) using 7-bromo-3-chloroisoquinolin-1-amine (46b) (81 mg, 0.314 mmol), K₃PO₄ (2M aqueous solution, 0.418 mL, 0.836 mmol), PCy₃ (11.72 mg, 0.042 mmol), Pd₂(dba)₃ (19.14 mg, 0.021 mmol). PdCl₂(dppf)-CH₂Cl₂ adduct (17.1 mg, 0.021 mmol) and heating at 80° ° C. for 1 h on oil bath. This gave after workup and purification using method-U, ethyl 2-(2-((5-(1-amino-3-chloroisoquinolin-7-yl)-1-isopropyl-1H-indazol-3-yl)methoxy)phenyl)acetate (46c) (60 mg, 54.3% yield) as a clear oil; ¹H NMR (300 MHz, DMSO-d₆) δ 8.54 (d, J=1.6 Hz, 1H), 8.15 (d, J=1.5 Hz, 1H), 8.06 (dd, J=8.6, 1.6 Hz, 1H), 7.93 (dd, J=8.9, 1.6 Hz, 1H), 7.86 (d, J=8.9 Hz, 1H), 7.75 (d, J=8.6 Hz, 1H), 7.44 (s, 2H), 7.30 (d, J=4.0 Hz, 2H), 7.19 (d, J=7.2 Hz, 1H), 7.00 (s, 1H), 6.92 (dt, J=7.3, 4.2 Hz, 1H), 5.44 (s, 2H), 5.07 (p, J=6.6 Hz, 1H), 3.61 (q, J=7.1 Hz, 2H), 3.53 (s, 2H), 1.52 (d, J=6.6 Hz, 6H), 0.71 (t, J=7.1 Hz, 3H); MS (ES+): 529.20 & 531.20 (M+1); (ES−): 527.05 (M−1).

### Step-3: Preparation of 2-(2-((5-(1-amino-3-chloroisoquinolin-7-yl)-1-isopropyl-1H-indazol-3-yl)methoxy)phenyl)acetic acid (46d)

Compound 46d was prepared according to the procedure and method of purification reported in step-2 of scheme 1, from ethyl 2-(2-((5-(1-amino-3-chloroisoquinolin-7-yl)-1-isopropyl-1H-indazol-3-yl)methoxy)phenyl)acetate (46c) (60 mg, 0.113 mmol) in THE/MeOH (4 mL, 1:1) using a solution of LiOH·H₂O (28.6 mg, 0.68 mmol) in water (I mL) to afford after workup and purification using method-G, 2-(2-((5-(1-amino-3-chloroisoquinolin-7-yl)-1-isopropyl-1H-indazol-3-yl)methoxy)phenyl)acetic acid (46d) (26 mg, 45.8% yield) HCl salt as a white solid; ¹H NMR (300 MHz, DMSO-d₆) δ 8.59 (s, 1H), 8.20 (d, J=1.6 Hz, 1H), 8.10 (dd, J=8.6, 1.6 Hz, 1H), 7.94 (dd, J=8.9, 1.6 Hz, 1H), 7.86 (d, J=8.9 Hz, 1H), 7.77 (d, J=8.6 Hz, 1H), 7.27 (dd, J=6.0, 1.7 Hz, 2H), 7.23-7.15 (m, 1H), 7.05 (s, 1H), 6.92 (td, J=6.7, 2.2 Hz, 1H), 5.46 (s, 2H), 5.15-4.95 (m, 1H), 3.53 (s, 2H), 1.52 (d, J=6.6 Hz, 6H); MS (ES+): 501.15 & 503.10 (M+1); (ES−): 499.10 & 501.10 (M−1).

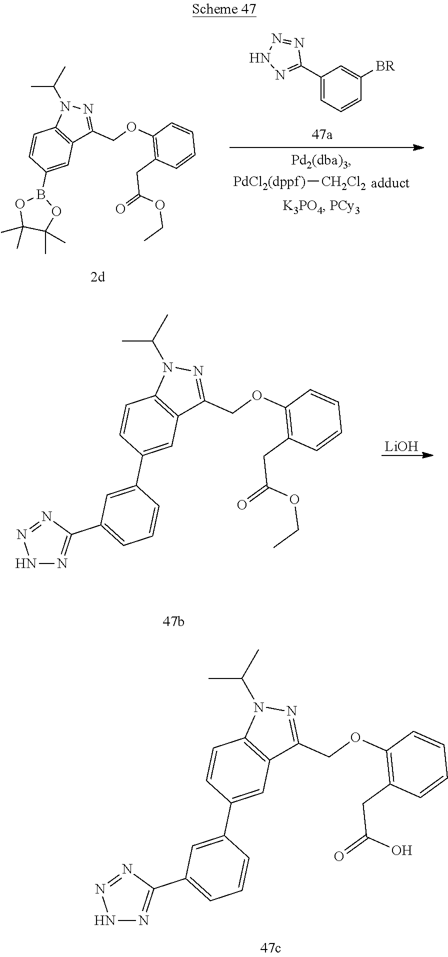

### Preparation of 2-(2-((5-(3-(2H-tetrazol-5-yl)phenyl)-1-isopropyl-1H-indazol-3-yl)methoxy)phenyl)acetic acid (47c)

### Step-1: Preparation of ethyl 2-(2-((5-(3-(2H-tetrazol-5-yl)phenyl)-1-isopropyl-1H-indazol-3-yl)methoxy)phenyl)acetate (47b)

Compound 47b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1), using 5-(3-bromophenyl)-2H-tetrazole (47a) (188 mg, 0.836 mmol; CAS #3440-99-1), K₃PO₄ (2M aqueous solution, 0.836 mL, 1.672 mmol), PCy₃ (23.45 mg, 0.084 mmol). Pd₂(dba)₃ (38.3 mg, 0.042 mmol) and PdCl₂(dppf)-CH₂Cl₂ adduct (34.1 mg, 0.042 mmol) to afford after workup and purification using method-U, ethyl 2-(2-((5-(3-(2H-tetrazol-5-yl)phenyl)-1-isopropyl-1H-indazol-3-yl)methoxy)phenyl)acetate (47b) (100 mg, 48.2% yield) as a clear oil; ¹H NMR (300 MHz, DMSO-d₆) δ 8.37 (d, J=1.7 Hz, 1H), 8.10 (s, 1H), 8.01 (d, J=7.6 Hz, 1H), 7.97-7.84 (m, 2H), 7.84-7.77 (m, 1H), 7.68 (t, J=7.8 Hz, 2H), 7.29 (d, J=4.0 Hz, 2H), 7.19 (d, J=7.4 Hz, 1H), 6.92 (dt, J=7.6, 4.2 Hz, 1H), 5.45 (s, 2H), 5.06 (p, J=6.7 Hz, 1H), 3.62 (q, J=7.1 Hz, 2H), 3.53 (s, 2H), 1.52 (d, J=6.6 Hz, 6H), 0.73 (t, J=7.1 Hz, 3H); MS (ES+): 497.20 (M+1); (ES−): 495.20 (M−1).

### Step-2: Preparation of 2-(2-((5-(3-(2H-tetrazol-5-yl)phenyl)-1-isopropyl-1H-indazol-3-yl)methoxy)phenyl)acetic acid (47c)

Compound 47c was prepared according to the procedure and method of purification reported in step-2 of scheme 1, from ethyl 2-(2-((5-(3-(2H-tetrazol-5-yl)phenyl)-1-isopropyl-1H-indazol-3-yl)methoxy)phenyl)acetate (47b) (100 mg, 0.201 mmol) in THE/MeOH (4 mL, 1:1) using a solution of LiOH·H₂O (50.7 mg, 1.208 mmol) in water (I mL) to afford after workup and purification using method-G, 2-(2-((5-(3-(2H-tetrazol-5-yl)phenyl)˜1-isopropyl-1H-indazol-3-yl)methoxy)phenyl)acetic acid (47c) (15 mg, 15.90% yield) HCl salt as an orange solid; ¹H NMR (300 MHz, DMSO-d₆) δ 8.41-8.34 (m, 1H), 8.15 (s, 1H), 8.04-7.99 (m, 1H), 7.96 (d, J=8.0 Hz, 1H), 7.88 (d, J=8.8 Hz, 1H), 7.81 (dd, J=8.8, 1.7 Hz, 1H), 7.70 (t, J=7.8 Hz, 1H), 7.33-7.23 (m, 2H), 7.23-7.16 (m, 1H), 6.96-6.87 (m, 1H), 5.48 (s, 2H), 5.14-4.96 (m, 1H), 3.53 (s, 2H), 1.53 (d, J=6.6 Hz, 6H); MS (ES+): 469.20 (M+1); (ES−): 467.10 (M−1).

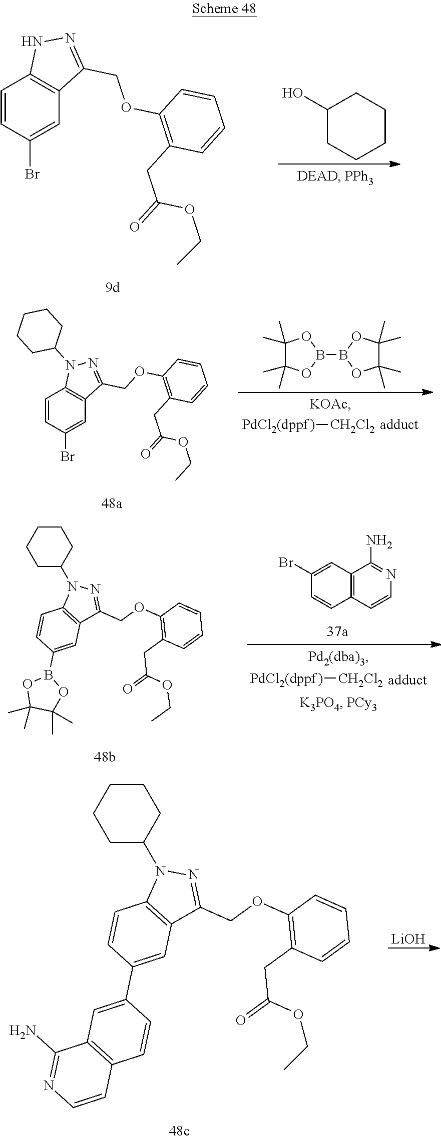

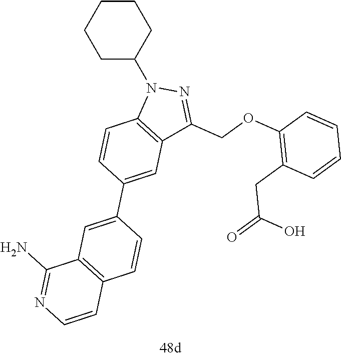 [SMILES: Nc1nccc2ccc(-c3ccc4c(c3)c(COc3ccccc3CC(=O)O)nn4C3CCCCC3)cc12]

### Preparation of 2-(2-((5-(1-aminoisoquinolin-7-yl)-1-cyclohexyl-1H-indazol-3-yl)methoxy)phenyl)acetic acid (48d)

### Step-1: Preparation of ethyl 2-(2-((5-bromo-1-cyclohexyl-1H-indazol-3-yl)methoxy)phenyl)acetate (48a)

Compound 48a was prepared according to the procedure and method of purification reported in step-1 of scheme 8, from ethyl 2-(2-((5-bromo-1H-indazol-3-yl)methoxy)phenyl)acetate (9d) (1000 mg, 2.57 mmol) in THF, using DEAD (895 mg, 5.14 mmol), PPh₃ (1348 mg, 5.14 mmol) and cyclohexanol (515 mg, 5.14 mmol) to afford after workup and purification using method-N, ethyl 2-(2-((5-bromo-1-cyclohexyl-1H-indazol-3-yl)methoxy)phenyl)acetate (48a) (210 mg, 17.34% yield) as a clear oil; 1H NMR (300 MHz, DMSO-d₆) δ 7.93 (d, J=1.8 Hz, 1H), 7.76 (d, J=9.0 Hz, 1H), 7.51 (dd, J=8.9, 1.9 Hz, 1H), 7.34-7.12 (m, 3H), 6.92 (td, J=7.1, 1.5 Hz, 1H), 5.36 (s, 2H), 4.62 (p, J=5.2, 4.8 Hz, 1H), 3.91 (q, J=7.1 Hz, 2H), 3.53 (s, 2H), 2.01-1.78 (m, 6H), 1.78-1.62 (m, 1H), 1.61-1.38 (m, 2H), 1.37-1.11 (m, 1H), 0.94 (t, J=7.1 Hz, 3H); MS (ES+): 471.10 & 473.05 (M+1).

### Step-2: Preparation of ethyl 2-(2-((1-cyclohexyl-5-(4,4,5,5-tetramethyl-1,3,2-dioxaborolan-2-yl)-1H-indazol-3-yl)methoxy)phenyl)acetate (48b)

Compound 48b was prepared according to the procedure and method of purification reported in step-4 of scheme 1, from ethyl 2-(2-((5-bromo-1-cyclohexyl-1H-indazol-3-yl)methoxy)phenyl)acetate (48a) (200 mg, 0.424 mmol) in anhydrous dioxane (10 mL), using BISPIN (215 mg, 0.849 mmol), KOAc (104 mg, 1.061 mmol), PdCl₂(dppf)-CH₂Cl₂ adduct (20.79 mg, 0.025 mmol) and heating for 3h at 100° C. This gave after workup and purification using method-C, ethyl 2-(2-((1-cyclohexyl-5-(4,4,5,5-tetramethyl-1,3,2-dioxaborolan-2-yl)-1H-indazol-3-yl)methoxy)phenyl)acetate (48b) (130 mg, 59.1% yield) as a white oil; 1H NMR (300 MHz, DMSO-d₆) δ 8.15 (d, J=1.0 Hz, 1H), 7.71 (dd, J=8.6, 0.9 Hz, 1H), 7.64 (dd, J=8.6, 1.0 Hz, 1H), 7.34-7.23 (m, 2H), 7.23-7.14 (m, 1H), 6.98-6.86 (m, 1H), 5.38 (s, 2H), 4.60 (dd, J=9.9, 4.7 Hz, 1H), 3.86 (q, J=7.1 Hz, 2H), 3.51 (s, 2H), 1.98-1.78 (m, 6H), 1.78-1.62 (m, 1H), 1.62-1.37 (m, 3H), 1.29 (s, 12H), 0.88 (t, J=7.1 Hz, 3H); MS (ES+): 519.30 (M+1).

### Step-3: Preparation of ethyl 2-(2-((5-(1-aminoisoquinolin-7-yl)-1-cyclohexyl-1H-indazol-3-yl)methoxy)phenyl)acetate (48c)

Compound 48c was prepared according to the procedure and method of purification reported in step-5 of scheme 1, from ethyl 2-(2-((1-cyclohexyl-5-(4,4,5,5-tetramethyl-1,3,2-dioxaborolan-2-yl)-1H-indazol-3-yl)methoxy)phenyl)acetate (48b) (130 mg, 0.21 mmol) in dioxane/THF (4 mL, 1:1), using 7-bromoisoquinolin-1-amine (37a) (67.1 mg, 0.301 mmol), K₃PO₄ (2M aqueous solution, 0.501 mL, 1.003 mmol), PCy₃ (14.06 mg, 0.05 mmol), Pd₂(dba)₃ (22.96 mg, 0.025 mmol) and PdCl₂(dppf)-CH₂Cl₂ adduct (20.5 mg, 0.025 mmol) to afford after workup and purification using method-AE, ethyl 2-(2-((5-(1-aminoisoquinolin-7-yl)-1-cyclohexyl-1H-indazol-3-yl)methoxy)phenyl)acetate (48c) (100 mg, 74.6% yield) as a clear oil; 1H NMR (300 MHz, DMSO-d₆) δ 8.53 (s, 1H), 8.15 (d, J=1.4 Hz, 1H), 8.08-7.99 (m, 1H), 7.98-7.91 (m, 1H), 7.88 (d, J=8.9 Hz, 1H), 7.80-7.74 (m, 2H), 7.30 (d, J=4.1 Hz, 2H), 7.19 (d, J=7.3 Hz, 1H), 6.95 (d, J=5.3 Hz, 2H), 6.91 (q, J=4.3, 3.3 Hz, 2H), 5.44 (s, 2H), 4.68 (t, J=5.4 Hz, 1H), 3.62 (q, J=7.1 Hz, 2H), 3.53 (s, 2H), 1.79-1.74 (m, 6H), 1.27-1.23 (m, 4H), 0.71 (t, J=7.1 Hz, 3H); MS (ES+): 535.30 (M+1).

### Step-4: Preparation of Preparation of 2-(2-((5-(1-aminoisoquinolin-7-yl)-1-cyclohexyl-1H-indazol-3-yl)methoxy)phenyl)acetic acid (48d)

Compound 48d was prepared according to the procedure and method of purification reported in step-2 of scheme 1, from ethyl 2-(2-((5-(1-aminoisoquinolin-7-yl)-1-cyclohexyl-1H-indazol-3-yl)methoxy)phenyl)acetate (48c) (100 mg, 0.187 mmol) in THE/MeOH (4 mL, 1:1) using a solution of LiOH·H₂O (47.1 mg, 1.122 mmol) in water (1 mL) to afford after workup and purification using method-G, 2-(2-((5-(1-aminoisoquinolin-7-yl)-1-cyclohexyl-1H-indazol-3-yl)methoxy)phenyl)acetic acid (48d) (55 mg, 0.109 mmol, 58.0% yield) HCl salt as a light yellow solid; 1H NMR (300 MHz, DMSO-d₆) δ 13.44 (s, 1H, D₂O exchangeable), 9.25 (s, 2H, D₂O exchangeable), 8.98 (s, 1H), 8.41 (dd, J=8.5, 1.6 Hz, 1H), 8.30 (s, 1H), 8.09-7.96 (m, 2H), 7.93 (d, J=8.9 Hz, 1H), 7.69 (d, J=6.9 Hz, 1H), 7.31-7.23 (m, 3H), 7.23-7.16 (m, 1H), 6.97-6.86 (m, 1H), 5.47 (s, 2H), 4.77-4.59 (m, 1H), 3.53 (s, 2H), 2.03-1.81 (m, 6H), 1.79-1.67 (m, 1H), 1.64-1.43 (m, 2H), 1.40-1.16 (m, 1H); MS (ES+): 507.20 (M+1); (ES−): 505.20 (M−1).

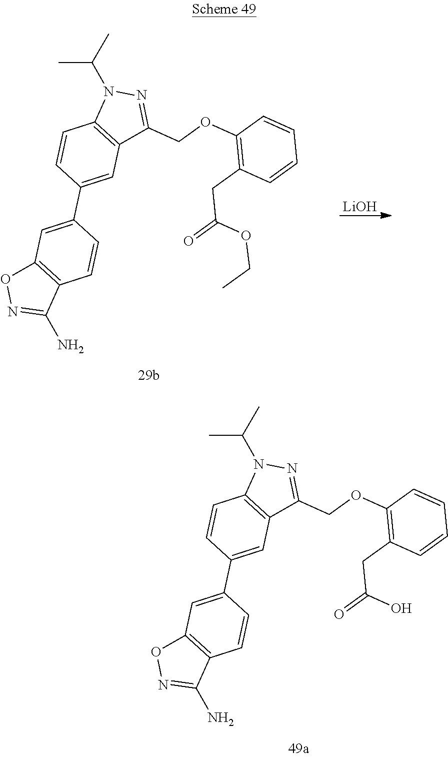 [SMILES: CCOC(=O)Cc1ccccc1OCc1nn(C(C)C)c2ccc(-c3ccc4c(N)noc4c3)cc12 ; "LiOH" ; CC(C)n1nc(COc2ccccc2CC(=O)O)c2cc(-c3ccc4c(N)noc4c3)ccc21]

### Preparation of 2-(2-((5-(3-aminobenzo[d]isoxazol-6-yl)-1-isopropyl-1H-indazol-3-yl)methoxy)phenyl)acetic acid (49a)

Compound 49a was prepared according to the procedure and method of purification reported in step-2 of scheme 1, from ethyl 2-(2-((5-(3-aminobenzo[d]isoxazol-6-yl)-1-isopropyl-1H-indazol-3-yl)methoxy)phenyl)acetate (29b) (20 mg, 0.041 mmol) in THF/MeOH (4 mL, 1:1) using a solution of LiOH·H₂O (5.2 mg, 0.124 mmol) in water (1 mL) and stirring at RT for 15 h. This gave after workup and purification using method-G, 2-(2-((5-(3-aminobenzo[d]isoxazol-6-yl)-1-isopropyl-1H-indazol-3-yl)methoxy)phenyl)acetic acid (49a) (5 mg, 26.5% yield) HCl salt as a white solid; ¹H NMR (300 MHz, DMSO-d₆) δ 8.15 (s, 1H), 7.88 (d, J=8.2 Hz, 1H), 7.81 (s, 2H), 7.76 (s, 1H), 7.62 (dd, J=8.2, 1.4 Hz, 1H), 7.32-7.22 (m, 2H), 7.22-7.16 (m, 1H), 6.96-6.87 (m, 1H), 5.46 (s, 2H), 5.11-4.96 (m, 1H), 3.52 (s, 2H), 1.52 (d, J=6.5 Hz, 6H); MS (ES+): 457.20 (M+1); (ES−): 455.10 (M−1).

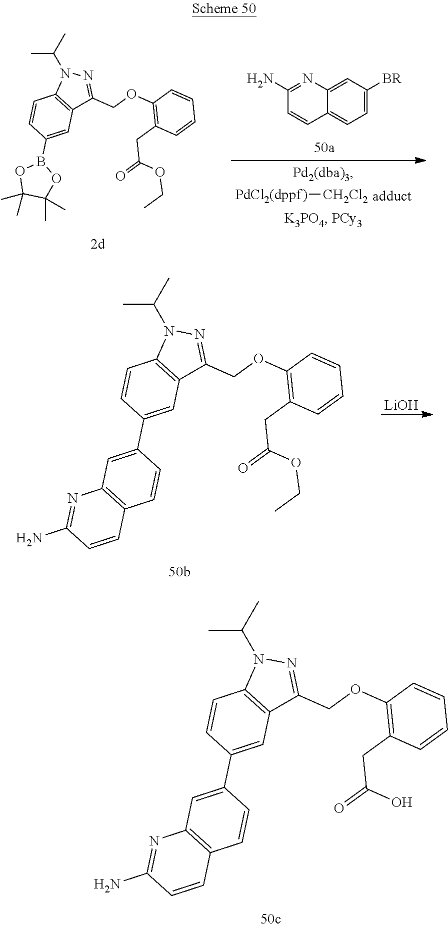

### Preparation of 2-(2-((5-(2-aminoquinolin-7-yl)-1-isopropyl-1H-indazol-3-yl)methoxy)phenyl)acetic acid (50c)

### Step-1: Preparation of ethyl 2-(2-((5-(2-aminoquinolin-7-yl)-1-isopropyl-1H-indazol-3-yl)methoxy)phenyl)acetate (50b)

Compound 50b was prepared according to the procedure and method of purification reported in step-5 of scheme 1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (100 mg, 0.209 mmol) in dioxane/THF (4 mL, 1:1) using 7-bromoquinolin-2-amine (50a) (69.9 mg, 0.314 mmol; CAS #116632-53-2), K₃PO₄ (2M aqueous solution, 0.418 mL, 0.836 mmol), PCy₃ (11.72 mg, 0.042 mmol), Pd₂(dba)₃ (19.14 mg, 0.021 mmol) and PdCl₂(dppf)-CH₂Cl₂ adduct (17.1 mg, 0.021 mmol) to afford after workup and purification using method-U, ethyl 2-(2-((5-(2-aminoquinolin-7-yl)-1-isopropyl-1H-indazol-3-yl)methoxy)phenyl)acetate (50b) (60 mg, 58.0% yield) as a clear oil; MS (ES+): 495.25 (M+1).

### Step-2: Preparation of 2-(2-((5-(2-aminoquinolin-7-yl)-1-isopropyl-1H-indazol-3-yl)methoxy)phenyl)acetic acid (50c)

Compound 50c was prepared according to the procedure and method of purification reported in step-2 of scheme 1, from ethyl 2-(2-((5-(2-aminoquinolin-7-yl)-1-isopropyl-1H-indazol-3-yl)methoxy)phenyl)acetate (50b) (60 mg, 0.121 mmol) in THE/MeOH (4 mL, 1:1) using a solution of LiOH·H₂O (30.5 mg, 0.728 mmol) in water (1 mL) to afford after workup and purification using method-G, 2-(2-((5-(2-aminoquinolin-7-yl)-1-isopropyl-1H-indazol-3-yl)methoxy)phenyl)acetic acid (50c) (52 mg, 0.111 mmol, 92% yield) HCl salt as a white solid; ¹H NMR (300 MHz, DMSO-d₆) δ 8.40 (d, J=9.3 Hz, 1H), 8.19 (s, 1H), 8.04-7.94 (m, 2H), 7.93-7.85 (m, 2H), 7.81 (dd, J=8.9, 1.7 Hz, 1H), 7.31-7.23 (m, 2H), 7.20 (d, J=7.4 Hz, 1H), 7.09 (d, J=9.3 Hz, 1H), 6.97-6.87 (m, 1H), 5.48 (s, 2H), 5.14-4.96 (m, 1H), 3.53 (s, 2H), 1.53 (d, J=6.5 Hz, 6H); MS (ES+): 467.20 (M+1); (ES−): 465.20 (M−1).

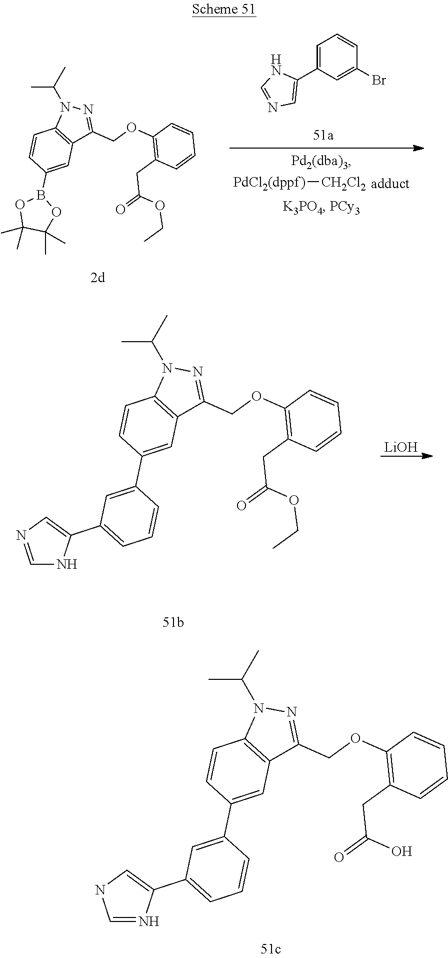

### Preparation of 2-(2-((5-(3-(1H-imidazol-5-yl)phenyl)-1-isopropyl-1H-indazol-3-yl)methoxy)phenyl)acetic acid (51c)

### Step-1: Preparation of ethyl 2-(2-((5-(3-(1H-imidazol-5-yl)phenyl)-1-isopropyl-1H-indazol-3-yl)methoxy)phenyl)acetate (51b)

Compound 51b was prepared according to the procedure and method of purification reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (100 mg, 0.209 mmol) in dioxane/THF (4 mL, 1:1) using 5-(3-bromophenyl)-1H-imidazole (51a) (69.9 mg, 0.314 mmol; CAS #53848-00-3), K₃PO₄ (2M aqueous solution, 0.418 mL, 0.836 mmol), PCy₃ (11.72 mg, 0.042 mmol). Pd₂(dba)₃ (19.14 mg, 0.021 mmol) and Pd(dppf)Cl₂—CH₂Cl₂ adduct (17.1 mg, 0.021 mmol) to afford after workup and purification using method-U, ethyl 2-(2-((5-(3-(1H-imidazol-5-yl)phenyl)-1-isopropyl-1H-indazol-3-yl)methoxy)phenyl)acetate (51b) (60 mg, 58.0% yield) as a clear oil; MS (ES+): 495.20 (M+1); (ES−): 493.20 (M−1).

### Step-2: Preparation of 2-(2-((5-(3-(1H-imidazol-5-yl)phenyl)-1-isopropyl-1H-indazol-3-yl)methoxy)phenyl)acetic acid (51c)

Compound 51c was prepared according to the procedure and method of purification reported in step-2 of scheme-1, from ethyl 2-(2-((5-(3-(1H-imidazol-5-yl)phenyl)-1-isopropyl-1H-indazol-3-yl)methoxy)phenyl)acetate (51b) (60 mg, 0.121 mmol) in THF/MeOH (4 mL, 1:1) using a solution of LiOH·H₂O (30.5 mg, 0.728 mmol) in water (1 mL) to afford after workup and purification using method-G, 2-(2-((5-(3-(1H-imidazol-5-yl)phenyl)-1-isopropyl-1H-indazol-3-yl)methoxy)phenyl)acetic acid (51c) (10 mg, 0.021 mmol, 17.67% yield) HCl salt as a white solid; ¹H NMR (300 MHz, DMSO-d₆) δ 9.13 (s, 1H), 8.28-8.19 (m, 2H), 8.14 (s, 1H), 7.85 (s, 2H), 7.79 (t, J=7.1 Hz, 2H), 7.59 (t, J=7.7 Hz, 1H), 7.32-7.23 (m, 2H), 7.20 (d, J=7.4 Hz, 1H), 6.92 (t, J=7.2 Hz, 1H), 5.46 (s, 2H), 5.17-4.94 (m, 1H), 3.54 (s, 2H), 1.52 (d, J=6.5 Hz, 6H); MS (ES+): 467.20 (M+1); (ES−): 465.15 (M−1).

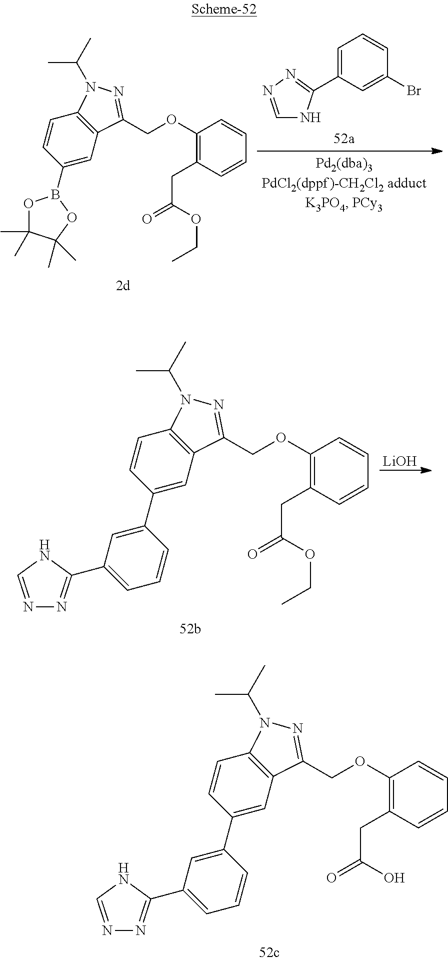

### Preparation of 2-(2-((5-(3-(4H-1,2,4-triazol-3-yl)phenyl)-1-isopropyl-1H-indazol-3-yl)methoxy)phenyl)acetic acid (52c)

### Step-1: Preparation of ethyl 2-(2-((5-(3-(4H-1,2,4-triazol-3-yl)phenyl)-1-isopropyl-1H-indazol-3-yl)methoxy)phenyl)acetate (52b)

Compound 52b was prepared according to the procedure and method of purification reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (100 mg, 0.209 mmol) in dioxane/THF (4 mL, 1:1) using 3-(3-bromophenyl)-4H-1,2,4-triazole (52a) (70.3 mg, 0.314 mmol; CAS #342617-08-7), K₃PO₄ (2M aqueous solution, 0.418 mL, 0.836 mmol), PCy₃ (11.72 mg, 0.042 mmol), Pd₂(dba)₃ (19.14 mg, 0.021 mmol) and Pd(dppf)Cl₂—CH₂Cl₂ adduct (17.1 mg, 0.021 mmol) to afford after workup and purification using method-U, ethyl 2-(2-((5-(3-(4H-1,2,4-triazol-3-yl)phenyl)-1-isopropyl-1H-indazol-3-yl)methoxy)phenyl)acetate (52b) (60 mg, 57.9% yield) as a clear oil; ¹H NMR (300 MHz, DMSO-d₆) δ 8.65 (s, 1H), 8.33 (s, 1H), 8.05 (d, J=8.4 Hz, 2H), 7.80 (d, J=14.9 Hz, 2H), 7.56 (d, J=8.1 Hz, 2H), 7.29 (d, J=4.2 Hz, 2H), 7.19 (d, J=7.4 Hz, 1H), 6.92 (s, TH), 5.45 (s, 2H), 5.02 (s, 1H), 3.64 (d, J=7.0 Hz, 2H), 3.53 (s, 2H), 1.52 (d, J=6.6 Hz, 6H), 0.73 (t, J=7.1 Hz, 3H); MS (ES+): 496.20 (M+1); (ES−): 494.20 (M−1).

### Step-2: Preparation of 2-(2-((5-(3-(4H-1,2,4-triazol-3-yl)phenyl)-1-isopropyl-1H-indazol-3-yl)methoxy)phenyl)acetic acid (52c)

Compound 52c was prepared according to the procedure and method of purification reported in step-2 of scheme-1, from ethyl 2-(2-((5-(3-(4H-1,2,4-triazol-3-yl)phenyl)-1-isopropyl-1H-indazol-3-yl)methoxy)phenyl)acetate (52b) (60 mg, 0.121 mmol) in THE/MeOH (4 mL, 1:1) using a solution of LiOH·H₂O (30.5 mg, 0.726 mmol) in water (1 mL) to afford after workup and purification using method-G, 2-(2-((5-(3-(4H-1,2,4-triazol-3-yl)phenyl)-1-isopropyl-1H-indazol-3-yl)methoxy)phenyl)acetic acid (52c) (4 mg, 7.07% yield) HCl salt as a white solid; ¹H NMR (300 MHz, DMSO-d₆) δ 8.83 (s, 1H, D₂O exchangeable), 8.53 (s, 1H), 8.37 (s, 1H), 8.09 (s, 1H), 7.99 (d, J=7.7 Hz, 1H), 7.88-7.73 (m, 4H), 7.59 (t, J=7.7 Hz, 1H), 7.33-7.24 (m, 2H), 7.21 (d, J=7.4 Hz, 1H), 6.97-6.85 (m, 1H), 5.48 (s, 2H), 5.11-4.96 (m, 1H), 3.56 (s, 2H), 1.53 (d, J=6.5 Hz, 6H); MS (ES+): 468.20 (M+1); (ES−): 466.10 (M−1).

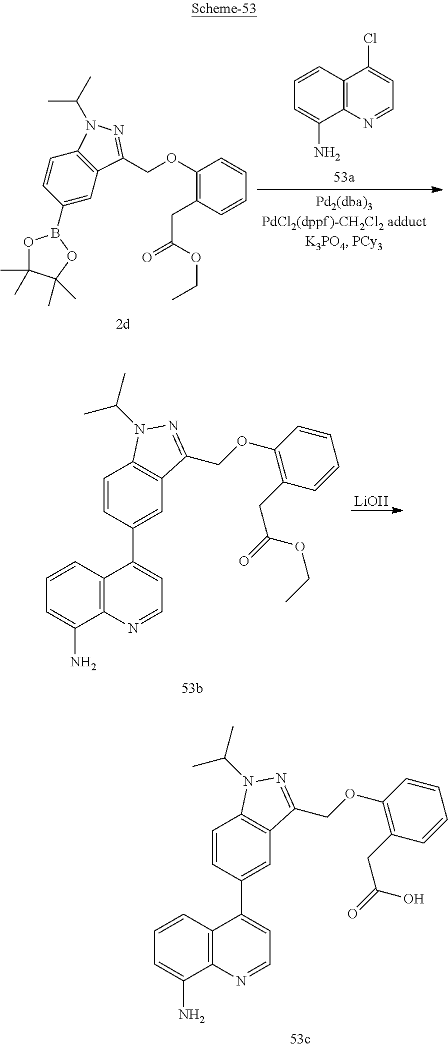

### Preparation of 2-(2-((5-(8-aminoquinolin-4-yl)-1-isopropyl-1H-indazol-3-yl)methoxy)phenyl)acetic acid (53c)

### Step-1: Preparation of ethyl 2-(2-((5-(8-aminoquinolin-4-yl)-1-isopropyl-1H-indazol-3-yl)methoxy)phenyl)acetate (53b)

Compound 53b was prepared according to the procedure and method of purification reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 4-chloroquinolin-8-amine (53a) (84 mg, 0.470 mmol; CAS #81764-16-1), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.7 mg, 0.031 mmol) to afford after workup and purification using method-U, ethyl 2-(2-((5-(8-aminoquinolin-4-yl)-1-isopropyl-1H-indazol-3-yl)methoxy)phenyl)acetate (53b) (100 mg, 64.5% yield) as a clear oil; ¹H NMR (300 MHz, DMSO-d₆) δ 8.76 (d, J=4.3 Hz, 1H), 7.88 (d, J=9.0 Hz, 2H), 7.56 (dd, J=8.6, 1.6 Hz, 1H), 7.42 (d, J=4.4 Hz, 1H), 7.32-7.21 (m, 3H), 7.16 (d, J=7.3 Hz, 1H), 7.05 (dd, J=8.4, 1.2 Hz, 1H), 6.94-6.84 (m, 2H), 6.01 (s, 2H), 5.43 (s, 2H), 5.08 (p, J=6.6 Hz, 1H), 3.58-3.45 (m, 4H), 1.54 (d, J=6.6 Hz, 6H), 0.67 (t, J=7.1 Hz, 3H); MS (ES+): 495.20 (M+1); (ES−): 493.00 (M−1).

### Step-2: Preparation of 2-(2-((5-(8-aminoquinolin-4-yl)-1-isopropyl-1H-indazol-3-yl)methoxy)phenyl)acetic acid (53c)

Compound 53c was prepared according to the procedure and method of purification reported in step-2 of scheme-1, from ethyl 2-(2-((5-(8-aminoquinolin-4-yl)-1-isopropyl-1H-indazol-3-yl)methoxy)phenyl)acetate (53b) (100 mg, 0.202 mmol) in THE/MeOH (4 mL, 1:1) using a solution of LiOH·H₂O (50.9 mg, 1.213 mmol) in water (1 mL) to afford after workup and purification using method-G, 2-(2-((5-(8-aminoquinolin-4-yl)-1-isopropyl-1H-indazol-3-yl)methoxy)phenyl)acetic acid (53c) (45 mg, 47.7% yield) HCl salt as an orange solid; ¹H NMR (300 MHz, DMSO-d₆) δ 8.98 (d, J=4.7 Hz, 1H), 8.03 (s, 1H), 7.93 (d, J=8.7 Hz, 1H), 7.67 (d, J=4.7 Hz, 1H), 7.60 (dd, J=8.8, 1.6 Hz, JH), 7.55-7.48 (m, 2H), 7.43 (dd, J=6.7, 2.4 Hz, 1H), 7.29-7.20 (m, 2H), 7.20-7.12 (m, 1H), 6.95-6.84 (m, 1H), 5.47 (s, 2H), 5.16-5.02 (m, 1H), 3.50 (s, 2H), 1.55 (d, J=6.6 Hz, 6H); MS (ES+): 467.20 (M+1); (ES−): 465.20 (M−1); Analysis calculated for C₂₈H₂₆N₄O₃·HCl·H₂O: C, 64.55; H, 5.61; Cl, 6.80; N, 10.75; Found: C, 64.35; H, 5.44; Cl, 6.62; N, 10.66.

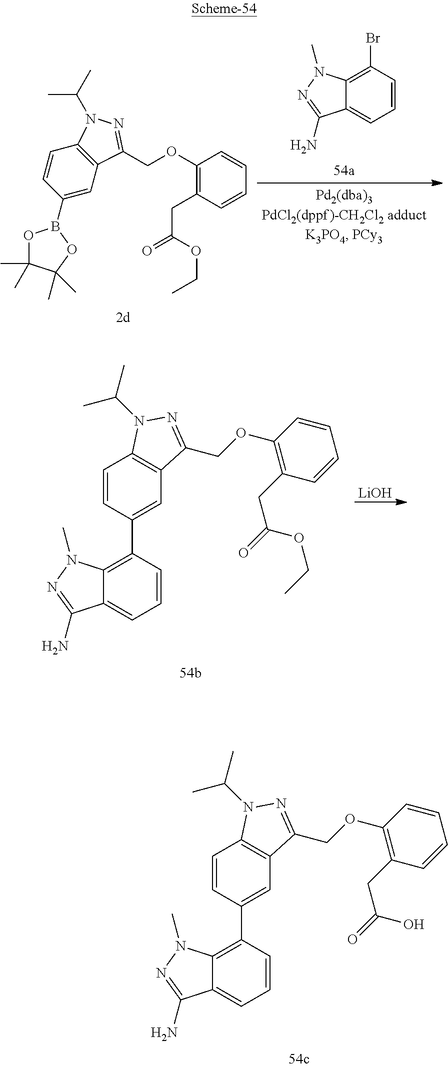

### Preparation of 2-(2-((3′-amino-1-isopropyl-1′-methyl-1H, 1′H-[5,7′-biindazol]-3-yl)methoxy)phenyl)acetic acid (54c)

### Step-1: Preparation of ethyl 2-(2-((3′-amino-1-isopropyl-1′-methyl-1H, 1′H-[5,7′-biindazol]-3-yl)methoxy)phenyl)acetate (54b)

Compound 54b was prepared according to the procedure and method of purification reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 7-bromo-1-methyl-1H-indazol-3-amine (54a) (106 mg, 0.470 mmol; CAS #1626335-95-2), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.7 mg, 0.031 mmol) to afford after workup and purification using method-U, ethyl 2-(2-((3′-amino-1-isopropyl-1′-methyl-1H, 1′H-5,7′-biindazol-3-yl)methoxy)phenyl)acetate (54b) (100 mg, 64.1% yield) as a clear oil; ¹H NMR (300 MHz, DMSO-d₆) δ 7.84-7.74 (m, 2H), 7.70 (dd, J=8.0, 1.1 Hz, 1H), 7.49 (dd, J=8.6, 1.6 Hz, 1H), 7.30-7.22 (m, 2H), 7.18-7.11 (m, 2H), 6.99 (dd, J=8.0, 7.0 Hz, 1H), 6.93-6.84 (m, 1H), 5.50 (s, 2H), 5.43 (s, 2H), 5.06 (p. J=6.5 Hz, 1H), 3.58 (q, J=7.1 Hz, 2H), 3.49 (s, 2H), 3.23 (s, 3H), 1.53 (d, J=6.6 Hz, 6H), 0.74 (t, J=7.1 Hz, 3H); MS (ES+): 498.30 (M+1).

### Step-2: Preparation of 2-(2-((3′-amino-1-isopropyl-1′-methyl-1H, 1′H-[5,7′-biindazol]-3-yl)methoxy)phenyl)acetic acid (54c)

Compound 54c was prepared according to the procedure and method of purification reported in step-2 of scheme-1, from ethyl 2-(2-((3′-amino-1-isopropyl-1′-methyl-1H, 1′H-5,7′-biindazol-3-yl)methoxy)phenyl)acetate (54b) (100 mg, 0.201 mmol) in THF/MeOH (4 mL, 1:1) using a solution of LiOH·H₂O (50.6 mg, 1.206 mmol) in water (1 mL) to afford after workup and purification using method-G, 2-(2-((3′-amino-1-isopropyl-1′-methyl-1H, 1H-[5,7′-biindazol]-3-yl)methoxy)phenyl)acetic acid (54c) (55 mg, 58.3% yield) HCl salt as a white solid; ¹H NMR (300 MHz, DMSO-d₆) δ 7.96-7.87 (m, 2H), 7.83 (d, J=8.7 Hz, 1H), 7.51 (dd, J=8.7, 1.6 Hz, 1H), 7.37 (dd, J=7.0, 1.1 Hz, 1H), 7.30-7.19 (m, 3H), 7.19-7.13 (m, 1H), 6.93-6.85 (m, 1H), 5.45 (s, 2H), 5.12-5.03 (m, 1H), 3.47 (s, 2H), 3.32 (s, 3H), 1.54 (d, J=6.6 Hz, 6H); MS (ES+): 470.20 (M+1); (ES−): 468.20 (M−1); Analysis calculated for C₂₇H₂₇N₅O₃·HCl, 1.25H₂O: C, 61.36; H, 5.82; Cl, 6.71; N, 13.25; Found: C, 61.47; H, 5.83; Cl, 6.38; N, 13.18.

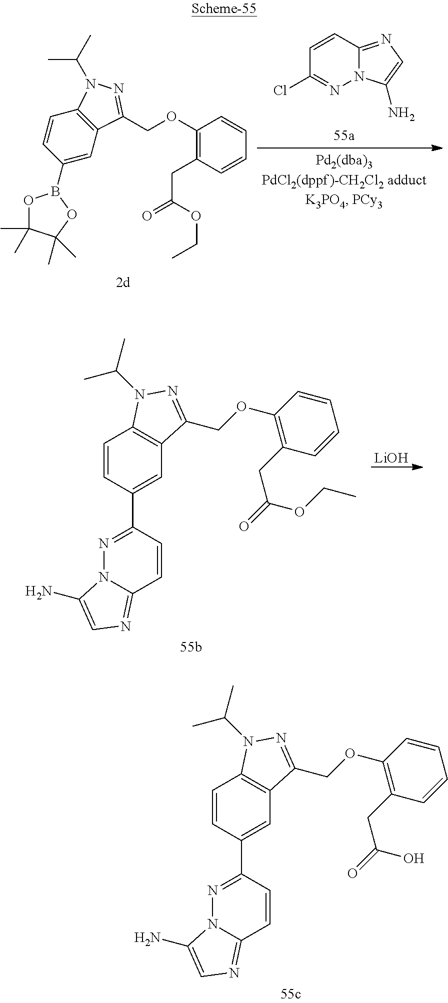

### Preparation of 2-(2-((5-(3-aminoimidazo[1,2-b]pyridazin-6-yl)-1-isopropyl-1H-indazol-3-yl)methoxy)phenyl)acetic acid (55c)

### Step-1: Preparation of ethyl 2-(2-((5-(3-aminoimidazo[1,2-b]pyridazin-6-yl)-1-isopropyl-1H-indazol-3-yl)methoxy)phenyl)acetate (55b)

Compound 55b was prepared according to the procedure and method of purification reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 6-chloroimidazo[1,2-b]pyridazin-3-amine (55a) (79 mg, 0.470 mmol; CAS #166176-45-0), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.7 mg, 0.031 mmol) to afford after workup and purification using method-U, ethyl 2-(2-((5-(3-aminoimidazo[1,2-b]pyridazin-6-yl)-1-isopropyl-1H-indazol-3-yl)methoxy)phenyl)acetate (55b) (120 mg, 79% yield) as a clear oil; ¹H NMR (300 MHz, DMSO-d₆) δ 8.49-8.44 (m, 1H), 8.30 (dd, J=8.9, 1.6 Hz, 1H), 7.97-7.84 (m, 2H), 7.54 (d, J=9.5 Hz, 1H), 7.30 (dd, J=4.6, 1.3 Hz, 2H), 7.20 (d, J=7.3 Hz, 1H), 7.04 (s, 1H), 6.98-6.89 (m, 1H), 5.57 (s, 2H), 5.46 (s, 2H), 5.08 (p, J=6.6 Hz, 1H), 3.73 (q, J=7.1 Hz, 2H), 3.55 (s, 2H), 1.52 (d, J=6.6 Hz, 6H), 0.75 (t, J=7.2 Hz, 3H); MS (ES+): 485.20 (M+1).

### Step-2: Preparation of 2-(2-((5-(3-aminoimidazo[1,2-b]pyridazin-6-yl)-1-isopropyl-1H-indazol-3-yl)methoxy)phenyl)acetic acid (55c)

Compound 55c was prepared according to the procedure and method of purification reported in step-2 of scheme-1, from ethyl 2-(2-((5-(3-aminoimidazo[1,2-b]pyridazin-6-yl)-1-isopropyl-1H-indazol-3-yl)methoxy)phenyl)acetate (55b) (120 mg, 0.248 mmol) in THF/MeOH (4 mL, 1:1) using a solution of LiOH·H₂O (62.4 mg, 1.486 mmol) in water (1 mL) to afford after workup and purification using method-G, 2-(2-((5-(3-aminoimidazo[1,2-b]pyridazin-6-yl)-1-isopropyl-1H-indazol-3-yl)methoxy)phenyl)acetic acid (55c) (30 mg, 26.5% yield) HCl salt as a yellow solid; 1H NMR (300 MHz, DMSO-d₆) δ 8.64 (d, J=1.7 Hz, 1H), 8.45 (dd, J=9.0, 1.7 Hz, 1H), 8.35 (d, J=9.7 Hz, 1H), 8.26 (d, J=9.7 Hz, 1H), 7.94 (d, J=9.1 Hz, 1H), 7.38 (s, 1H), 7.33-7.24 (m, 2H), 7.21 (d, J=7.3 Hz, 1H), 6.98-6.85 (m, 1H), 5.50 (s, 2H), 5.19-5.03 (m, 1H), 3.55 (s, 2H), 1.53 (d, J=6.5 Hz, 6H); MS (ES+): 457.20 (M+1); (ES−): 455.10 (M−1).

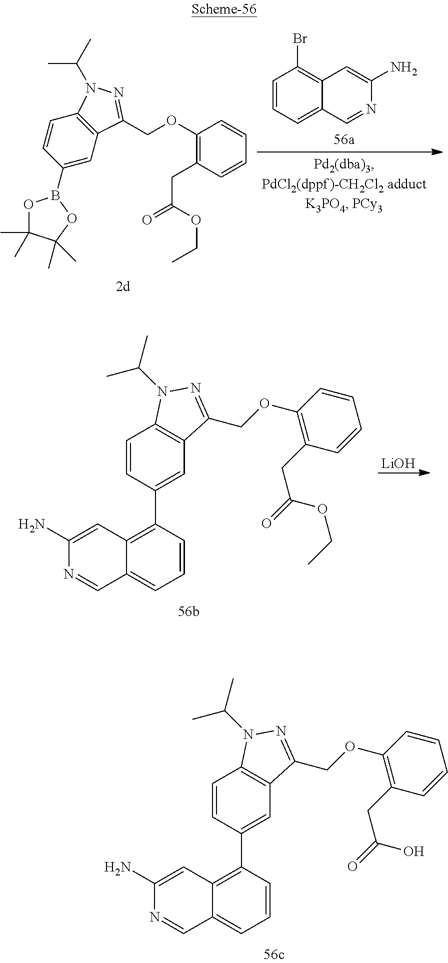

### Preparation of 2-(2-((5-(3-aminoisoquinolin-5-yl)-1-isopropyl-1H-indazol-3-yl)methoxy)phenyl)acetic acid (56c)

### Step-1: Preparation of ethyl 2-(2-((5-(3-aminoisoquinolin-5-yl)-1-isopropyl-1H-indazol-3-yl)methoxy)phenyl)acetate (56b)

Compound 56b was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 5-bromoisoquinolin-3-amine (56a) (105 mg, 0.47 mmol; CAS #1192815-01-2), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.7 mg, 0.031 mmol) to afford after workup and purification using method-U, ethyl 2-(2-((5-(3-aminoisoquinolin-5-yl)-1-isopropyl-1H-indazol-3-yl)methoxy)phenyl)acetate (56b) (120 mg, 77% yield) as a clear oil; 1H NMR (300 MHz, DMSO-d₆) δ 8.87 (s, 1H), 7.88-7.74 (m, 3H), 7.48 (dd, J=8.7, 1.6 Hz, 1H), 7.41 (dd, J=6.9, 1.3 Hz, 1H), 7.30-7.25 (m, 2H), 7.24-7.12 (m, 2H), 6.96-6.84 (m, 1H), 6.61 (s, 1H), 5.88 (s, 2H), 5.42 (s, 2H), 5.08 (p, J=6.6 Hz, 1H), 3.57-3.46 (m, 4H), 1.55 (d, J=6.5 Hz, 6H), 0.70 (t, J=7.1 Hz, 3H); MS (ES+): 495.25 (M+1); (ES−): 493.25 (M−1).

### Step-2: Preparation of 2-(2-((5-(3-aminoisoquinolin-5-yl)-1-isopropyl-1H-indazol-3-yl)methoxy)phenyl)acetic acid (56c)

Compound 56c was prepared according to the procedure reported in step-2 of scheme-1, from ethyl 2-(2-((5-(3-aminoisoquinolin-5-yl)-1-isopropyl-1H-indazol-3-yl)methoxy)phenyl)acetate (56b) (120 mg, 0.243 mmol) in THF/MeOH (4 mL, 1:1) using a solution of LiOH·H₂O (61.1 mg, 1.456 mmol) in water (I mL) to afford after workup and purification using method-G, 2-(2-((5-(3-aminoisoquinolin-5-yl)-1-isopropyl-1H-indazol-3-yl)methoxy)phenyl)acetic acid (56c) (55 mg, 48.6% yield) HCl salt as a yellow solid; ¹H NMR (300 MHz, DMSO-d₆) δ 9.22 (s, 1H), 8.04 (d, J=8.5 Hz, 1H), 7.93-7.85 (m, 2H), 7.68 (dd, J=7.0, 1.2 Hz, 1H), 7.52-7.40 (m, 2H), 7.30-7.22 (m, 2H), 7.19-7.13 (m, 1H), 7.09 (s, 1H), 6.89 (td, J=7.0, 1.9 Hz, 1H), 5.45 (s, 2H), 5.15-5.02 (m, 1H), 3.48 (s, 2H), 1.55 (d, J=6.6 Hz, 6H); MS (ES+): 467.20 (M+1); (ES−): 465.10 (M−1).

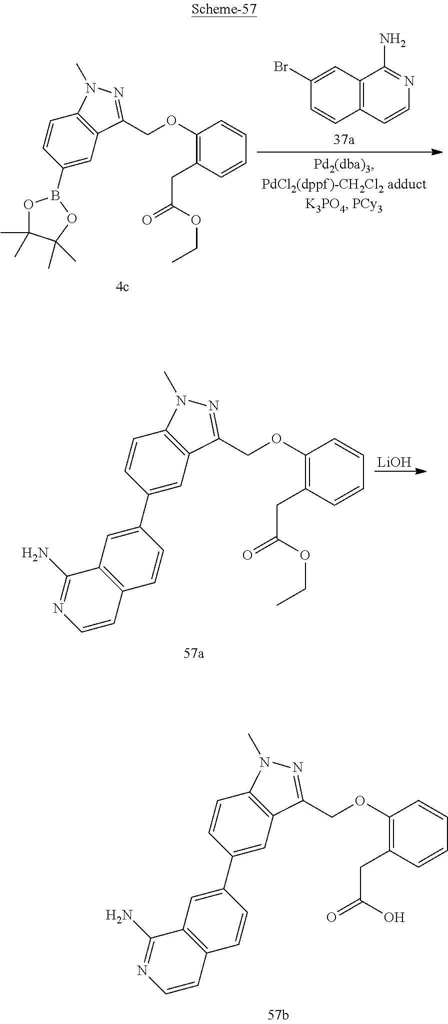

### Preparation of 2-(2-((5-(1-aminoisoquinolin-7-yl)-1-methyl-1H-indazol-3-yl)methoxy)phenyl)acetic acid (57b)

### Step-1: Preparation of ethyl 2-(2-((5-(1-aminoisoquinolin-7-yl)-1-methyl-1H-indazol-3-yl)methoxy)phenyl)acetate (57a)

Compound 57a was prepared according to the procedure reported in step-5 of scheme-1, from ethyl 2-(2-((1-methyl-5-(4,4,5,5-tetramethyl-1,3,2-dioxaborolan-2-yl)-1H-indazol-3-yl)methoxy)phenyl)acetate (4c) (131 mg, 0.586 mmol) in dioxane/THF (4 mL, 1:1) using 7-bromoisoquinolin-1-amine (37a) (131 mg, 0.586 mmol), K₃PO₄ (2M aqueous solution, 0.977 mL, 1.954 mmol), PCy₃ (27.4 mg, 0.098 mmol), Pd₂(dba)₃ (44.7 mg, 0.049 mmol) and Pd(dppf)Cl₂—CH₂Cl₂ adduct (39.9 mg, 0.049 mmol) to afford after workup and purification using method-AE, ethyl 2-(2-((5-(1-aminoisoquinolin-7-yl)-1-methyl-1H-indazol-3-yl)methoxy)phenyl)acetate (57a) (160 mg, 70.2% yield) as a clear oil; 1H NMR (300 MHz. DMSO-d₆) δ 8.58-8.51 (m, 1H), 8.17 (s, 1H), 8.05 (d, J=8.5 Hz, 1H), 7.97 (d, J=8.9 Hz, 1H), 7.81-7.78 (m, 2H), 7.29 (d, J=4.1 Hz, 2H), 7.19 (d, J=7.4 Hz, 1H), 6.95-6.91 (m, 3H), 5.44 (s, 2H), 4.10 (s, 3H), 3.61 (d, J=6.7 Hz, 2H), 3.52 (s, 2H), 0.73 (t, J=7.0 Hz, 3H); MS (ES+): 467.20 (M+1); (ES−): 465.25 (M−1).

### Step-2: Preparation of 2-(2-((5-(1-aminoisoquinolin-7-yl)-1-methyl-1H-indazol-3-yl)methoxy)phenyl)acetic acid (57b)

Compound 57b was prepared according to the procedure reported in step-6 of scheme-1, from ethyl 2-(2-((5-(1-aminoisoquinolin-7-yl)-1-methyl-1H-indazol-3-yl)methoxy)phenyl)acetate (57a) (160 mg, 0.343 mmol) in THE/MeOH (4 mL, 1:1) using a solution of LiOH·H₂O (86 mg, 2.058 mmol) in water (1 mL) to afford after workup and purification using method-G, 2-(2-((5-(1-aminoisoquinolin-7-yl)-1-methyl-1H-indazol-3-yl)methoxy)phenyl)acetic acid (57b) (65 mg, 0.148 mmol, 43.2% yield) HCl salt as a white solid; 1H NMR (300 MHz, DMSO-d₆) δ 13.30 (s, 1H, D₂O exchangeable), 12.10 (s, 1H, D₂O exchangeable), 9.19 (s, 2H, D₂O exchangeable), 8.96 (s, 1H), 8.42 (dd, J=8.5, 1.6 Hz, 1H), 8.32 (d, J=1.6 Hz, 1H), 8.08-7.98 (m, 2H), 7.85 (d, J=8.8 Hz, 1H), 7.69 (d, J=6.9 Hz, 1H), 7.31-7.24 (m, 3H), 7.20 (d, 1H), 6.99-6.88 (m, 1H), 5.47 (s, 2H), 4.11 (s, 3H), 3.52 (s, 2H); MS (ES+): 439.15 (M+1); (ES−): 437.10 (M−1).

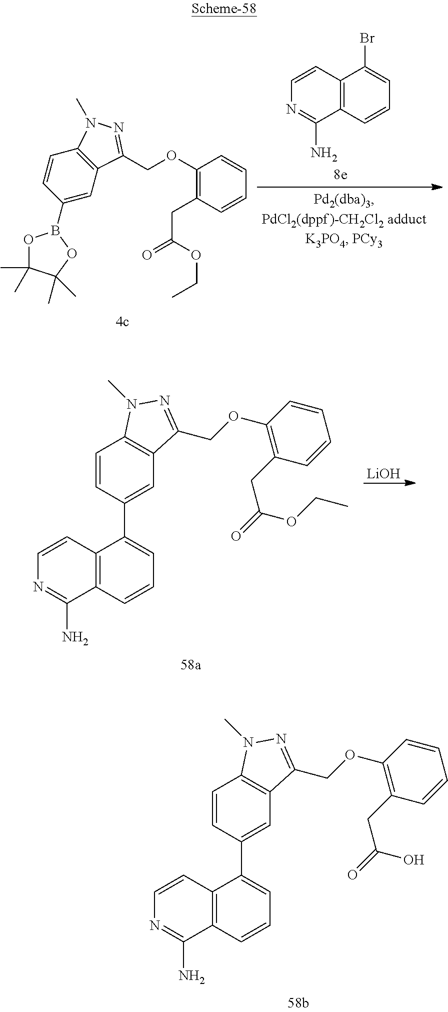

### Preparation of 2-(2-((5-(1-aminoisoquinolin-5-yl)-1-methyl-1H-indazol-3-yl)methoxy)phenyl)acetic acid (58b)

### Step-1: Preparation of ethyl 2-(2-((5-(1-aminoisoquinolin-5-yl)-1-methyl-1H-indazol-3-yl)methoxy)phenyl)acetate (58a)

Compound 58a was prepared according to the procedure reported in step-5 of scheme-1, from ethyl 2-(2-((1-methyl-5-(4,4,5,5-tetramethyl-1,3,2-dioxaborolan-2-yl)-1H-indazol-3-yl)methoxy)phenyl)acetate (4c) (220 mg, 0.489 mmol) in dioxane/THF (4 mL, 1:1) using 5-bromoisoquinolin-1-amine (5e) (131 mg, 0.586 mmol), K₃PO₄ (2M aqueous solution, 0.977 mL, 1.954 mmol), PCy₃ (27.4 mg, 0.098 mmol), Pd₂(dba)₃ (44.7 mg, 0.049 mmol) and Pd(dppf)Cl₂—CH₂Cl₂ adduct (39.9 mg, 0.049 mmol) to afford after workup and purification using method-AE, ethyl 2-(2-((5-(1-aminoisoquinolin-5-yl)-1-methyl-1H-indazol-3-yl)methoxy)phenyl)acetate (58a) (160 mg, 70.2% yield) as a clear oil; ¹H NMR (300 MHz, DMSO-d₆) δ 8.23 (dd, J=8.3, 4.6 Hz, 1H), 7.77 (dd, J=13.2, 5.0 Hz, 3H), 7.63-7.54 (m, 2H), 7.54-7.46 (m, 2H), 7.27-7.23 (m, 2H), 7.16 (d, J=7.4 Hz, 1H), 6.90 (d, J=4.5 Hz, 2H), 6.80 (d, J=6.1 Hz, 1H), 5.41 (s, 2H), 4.11 (s, 3H), 3.58-3.54 (m, 2H), 3.50 (s, 2H), 0.73 (t, J=7.1 Hz, 3H); MS (ES+): 467.20 (M+1); (ES−): 465.10 (M−1).

### Step-2: Preparation of 2-(2-((5-(1-aminoisoquinolin-5-yl)-1-methyl-1H-indazol-3-yl)methoxy)phenyl)acetic acid (58b)

Compound 58b was prepared according to the procedure reported in step-6 of scheme-1, from ethyl 2-(2-((5-(1-aminoisoquinolin-5-yl)-1-methyl-1H-indazol-3-yl)methoxy)phenyl)acetate (58a) (160 mg, 0.343 mmol) in THE/MeOH (4 mL, 1:1) using a solution of LiOH·H₂O (86 mg, 2.058 mmol) in water (1 mL) to afford after workup and purification using method-G, 2-(2-((5-(1-aminoisoquinolin-5-yl)-1-methyl-1H-indazol-3-yl)methoxy)phenyl)acetic acid (58b) (85 mg, 56.5% yield) HCl salt as a white solid; ¹H NMR (300 MHz, DMSO-d₆) δ 13.30 (s, 1H, D₂O exchangeable), 12.03 (s, 1H, D₂O exchangeable), 9.18 (s, 2H, D₂O exchangeable), 8.62 (d, J=8.3 Hz, 1H), 7.96 (dd, J=7.4, 1.1 Hz, 1H), 7.90-7.78 (m, 3H), 7.62 (d, J=7.3 Hz, 1H), 7.49 (dd, J=8.6, 1.6 Hz, 1H), 7.24 (d, J=4.1 Hz, 2H), 7.17 (d, J=7.3 Hz, 1H), 6.97 (d, J=7.2 Hz, 1H), 6.94-6.84 (m, 1H), 5.43 (s, 2H), 4.13 (s, 3H), 3.47 (s, 2H); MS (ES+): 439.20 (M+1); (ES−): 437.10 (M−1); Analysis calculated for C₂₆H₂₂NO₃·HCl·2H₂O: C, 61.12; H, 5.33; Cl, 6.94; N, 10.96; Found: C, 60.78; H, 5.23; Cl, 6.62; N, 10.75.

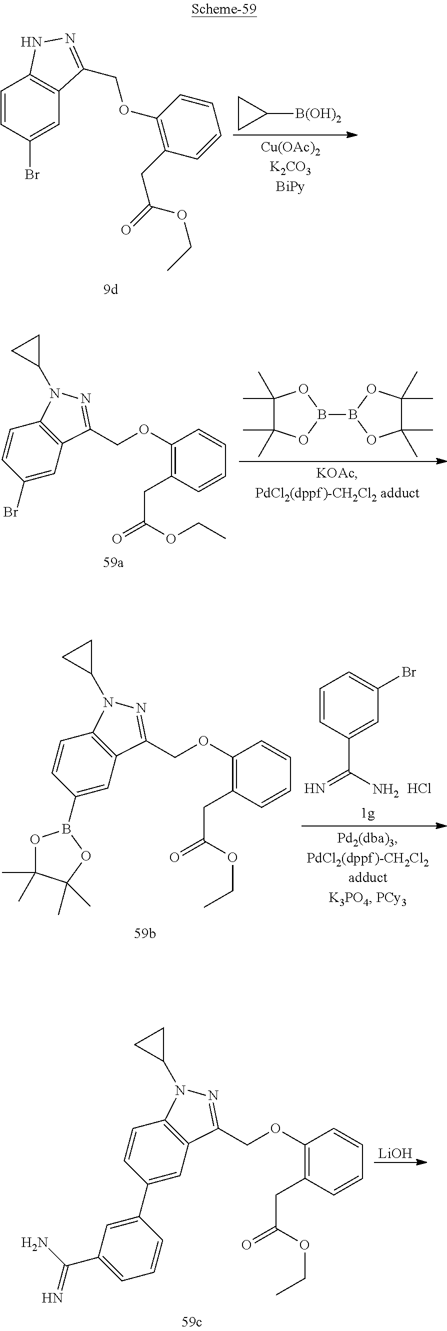

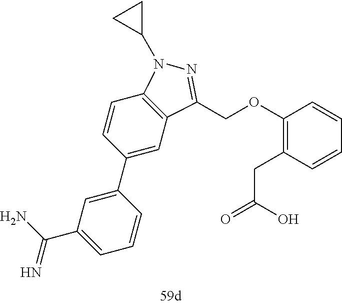 [SMILES: N=C(N)c1cccc(-c2ccc3c(c2)c(COc2ccccc2CC(=O)O)nn3C2CC2)c1]

### Preparation of 2-(2-((5-(3-carbamimidoylphenyl)-1-cyclopropyl-1H-indazol-3-yl)methoxy)phenyl)acetic acid (59d)

### Step-1: Preparation of ethyl 2-(2-((5-bromo-1-cyclopropyl-1H-indazol-3-yl)methoxy)phenyl)acetate (59a)

Compound 59a was prepared according to the procedure reported in step-1 of scheme-28, from ethyl 2-(2-((5-bromo-1H-indazol-3-yl)methoxy)phenyl)acetate (9d) (800 mg, 2.055 mmol) in DCE (20 mL) using cyclopropyl boronic acid (353 mg, 4.11 mmol), 2,2′-bipyridine (321 mg, 2.055 mmol), Cu(OAc)₂ (373 mg, 2.055 mmol) and K₂CO₃ (568 mg, 4.11 mmol) to afford after workup and purification using method-P, ethyl 2-(2-((5-bromo-1-cyclopropyl-1H-indazol-3-yl)methoxy)phenyl)acetate (59a) (450 mg, 51.0% yield) as a clear oil; ¹H NMR (300 MHz, DMSO-d₆) δ 7.95 (d, J=1.8 Hz, 1H), 7.69 (d, J=8.9 Hz, 1H), 7.57 (dd, J=8.9, 1.9 Hz, 1H), 7.32-7.15 (m, 3H), 6.92 (td, J=7.3, 1.3 Hz, 1H), 5.34 (s, 2H), 3.92 (q, J=7.1 Hz, 2H), 3.78 (tt, J=7.2, 4.0 Hz, 1H), 3.55 (s, 2H), 1.21-1.04 (m, 4H), 0.95 (t, J=7.1 Hz, 3H).

### Step-2: Preparation of ethyl 2-(2-((1-cyclopropyl-5-(4,4,5,5-tetramethyl-1,3,2-dioxaborolan-2-yl)-1H-indazol-3-yl)methoxy)phenyl)acetate (59b)

Compound 59b was prepared according to the procedure reported in step-4 of scheme-1, from ethyl 2-(2-((5-bromo-1-cyclopropyl-1H-indazol-3-yl)methoxy)phenyl)acetate (59a) (450 mg, 1.048 mmol) in anhydrous dioxane (20 mL), using BISPIN (532 mg, 2.096 mmol), KOAc (257 mg, 2.62 mmol), PdCl₂(dppf)-CH₂Cl₂Adduct (51.4 mg, 0.063 mmol) and heating for 3 h at 100° C. This gave after workup and purification method-C, ethyl 2-(2-((1-cyclopropyl-5-(4,4,5,5-tetramethyl-1,3,2-dioxaborolan-2-yl)-1H-indazol-3-yl)methoxy)phenyl)acetate) (59b) (170 mg, 34.0% yield) as a clear oil; 1H NMR (300 MHz, DMSO-d₆) δ 8.15 (d, J=1.1 Hz, 1H), 7.69 (d, J=1.2 Hz, 2H), 7.31-7.24 (m, 2H), 7.19 (d, J=7.3 Hz, 1H), 6.92 (td, J=7.2, 6.6, 1.9 Hz, 1H), 5.36 (s, 2H), 3.86 (q, J=7.1 Hz, 2H), 3.77 (dq, J=7.1, 3.5 Hz, 1H), 3.49 (d, J=11.1 Hz, 2H), 1.30 (s, 12H), 1.16-1.08 (m, 4H), 0.88 (t, J=7.1 Hz, 3H); MS (ES+): 477.20 (M+1); (ES−): 475.05 (M−1).

### Step-3: Preparation of ethyl 2-(2-((5-(3-carbamimidoylphenyl)-1-cyclopropyl-1H-indazol-3-yl)methoxy)phenyl)acetate (59c)

Compound 59c was prepared according to the procedure reported in step-5 of scheme-1, from ethyl 2-(2-((1-cyclopropyl-5-(4,4,5,5-tetramethyl-1,3,2-dioxaborolan-2-yl)-1H-indazol-3-yl)methoxy)phenyl)acetate (59b) (170 mg, 0.357 mmol) in dioxane/THF (6 mL, 1:1) using 3-bromobenzimidamide hydrochloride (1g) (168 mg, 0.714 mmol), K₃PO₄ (2M aqueous solution, 0.714 mL, 1.427 mmol), PCy₃ (20.01 mg, 0.071 mmol), Pd₂(dba)₃ (32.7 mg, 0.036 mmol). PdCl₂(dppf)-CH₂Cl₂ adduct (29.1 mg, 0.036 mmol) to afford after workup and purification using method-F, ethyl 2-(2-((5-(3-carbamimidoylphenyl)-1-cyclopropyl-1H-indazol-3-yl)methoxy)phenyl)acetate (59c) (100 mg, 59.8% yield) as a clear oil; MS (ES+): 469.20 (M+1).

### Step-4: Preparation of 2-(2-((5-(3-carbamimidoylphenyl)-1-cyclopropyl-1H-indazol-3-yl)methoxy)phenyl)acetic acid (59d)

Compound 59d was prepared according to the procedure reported in step-6 of scheme-1, from ethyl 2-(2-((5-(3-carbamimidoylphenyl)-1-cyclopropyl-1H-indazol-3-yl)methoxy)phenyl)acetate (59c) (100 mg, 0.213 mmol) in THE/MeOH (4 mL, 1:1) using a solution of LiOH·H₂O (53.7 mg, 1.281 mmol) in water (1 mL) to afford after workup and purification using method-G, 2-(2-((5-(3-carbamimidoylphenyl)-1-cyclopropyl-1H-indazol-3-yl)methoxy)phenyl)acetic acid (59d) (32 mg, 34.0% yield) HCl salt as a white solid; ¹H NMR (300 MHz, DMSO-d₆) δ 12.07 (s, 1H, D₂O exchangeable), 9.41 (s, 2H, D₂O exchangeable), 9.06 (s, 2H, D₂O exchangeable), 8.20 (s, 1H), 8.16-8.04 (m, 2H), 7.96-7.80 (m, 2H), 7.80-7.65 (m, 2H), 7.31-7.24 (m, 2H), 7.24-7.14 (m, 1H), 6.99-6.85 (m, 1H), 5.42 (s, 2H), 3.89-3.75 (m, 1H), 3.52 (s, 2H), 1.20-1.11 (m, 4H); MS (ES+): 441.20 (M+1); (ES−): 439.20 (M−1).

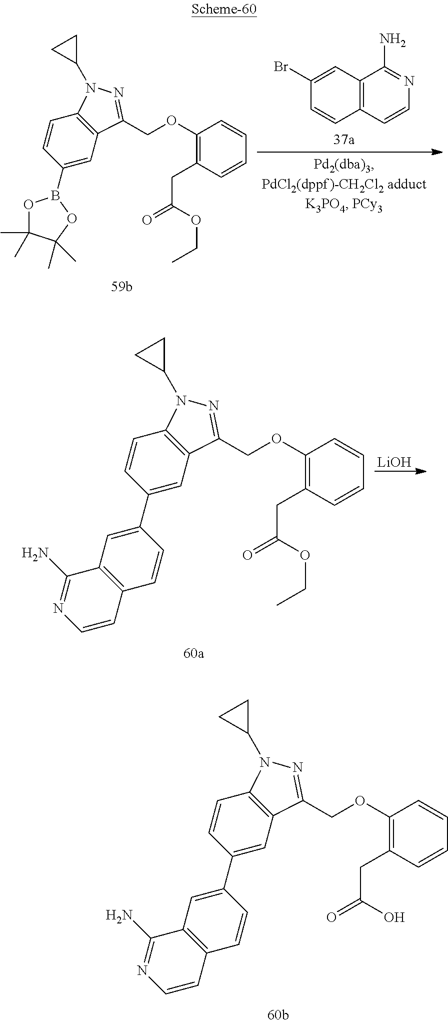

### Preparation of 2-(2-((5-(1-aminoisoquinolin-7-yl)-1-cyclopropyl-1H-indazol-3-yl)methoxy)phenyl)acetic acid (60b)

### Step-1: Preparation of ethyl 2-(2-((5-(1-aminoisoquinolin-7-yl)-1-cyclopropyl-1H-indazol-3-yl)methoxy)phenyl)acetate (60a)

Compound 60a was prepared according to the procedure reported in step-5 of scheme-1, from ethyl 2-(2-((1-cyclopropyl-5-(4,4,5,5-tetramethyl-1,3,2-dioxaborolan-2-yl)-1H-indazol-3-yl)methoxy)phenyl)acetate (59b) (112 mg, 0.504 mmol) in dioxane/THF (4 mL, 1:1) was added 7-bromoisoquinolin-1-amine (37a) (112 mg, 0.504 mmol), K₃PO₄ (2M aqueous solution, 0.84 mL, 1.679 mmol), PCy₃ (23.55 mg, 0.084 mmol), Pd₂(dba)₃ (38.4 mg, 0.042 mmol) and Pd(dppf)Cl₂—CH₂Cl₂ adduct (34.3 mg, 0.042 mmol) to afford after workup and purification using method-AE, ethyl 2-(2-((5-(1-aminoisoquinolin-7-yl)-1-cyclopropyl-1H-indazol-3-yl)methoxy)phenyl)acetate (60a) (150 mg, 72.5% yield) as a clear oil; 1H NMR (300 MHz, DMSO-d₆) δ 8.60 (s, 1H), 8.18 (s, 1H), 8.08 (dd, J=8.6, 1.6 Hz, 1H), 7.99 (dd, J=8.7, 1.6 Hz, 1H), 7.86-7.75 (m, 3H), 7.34-7.22 (m, 4H), 7.18 (d, J=7.4 Hz, 1H), 6.98 (d, J=6.0 Hz, 1H), 6.92 (td, J=6.1, 5.3, 2.7 Hz, 1H), 5.42 (s, 2H), 3.89-3.78 (m, 1H), 3.62 (q, J=7.1 Hz, 2H), 3.52 (s, 2H), 1.18-1.13 (m, 4H), 0.72 (t, J=7.1 Hz, 3H); MS (ES+): 493.20 (M+1); (ES−): 491.10 (M−1).

### Step-2: Preparation of 2-(2-((5-(1-aminoisoquinolin-7-yl)-1-cyclopropyl-1H-indazol-3-yl)methoxy)phenyl)acetic acid (60b)

Compound 60b was prepared according to the procedure reported in step-6 of scheme-1, from ethyl 2-(2-((5-(1-aminoisoquinolin-7-yl)-1-cyclopropyl-1H-indazol-3-yl)methoxy)phenyl)acetate (60a) (150 mg, 0.305 mmol) in THE/MeOH (4 mL, 1:1) using a solution of LiOH·H₂O (77 mg, 1.827 mmol) in water (1 mL) to afford after workup and purification using method-G, 2-(2-((5-(1-aminoisoquinolin-7-yl)-1-cyclopropyl-1H-indazol-3-yl)methoxy)phenyl)acetic acid (60b) (85 mg, 0.183 mmol, 60.1% yield) HCl salt as a white solid; 1H NMR (300 MHz, DMSO-d₆) δ 13.23 (s, 1H, D₂O exchangeable), 12.11 (s, 1H, DO exchangeable), 9.15 (s, 2H, D₂O exchangeable), 8.95 (s, 1H), 8.41 (dd, J=8.5, 1.6 Hz, 1H), 8.31 (s, 1H), 8.10-7.97 (m, 2H), 7.88 (d, J=8.8 Hz, 1H), 7.69 (d, J=6.9 Hz, 1H), 7.32-7.23 (m, 3H), 7.20 (d, J=7.3 Hz, 1H), 6.97-6.87 (m, 1H), 5.44 (s, 2H), 4.00-3.71 (m, 1H), 3.52 (s, 2H), 1.22-1.11 (m, 4H); MS (ES+): 465.20 (M+1); (ES−): 463.10 (M−1).

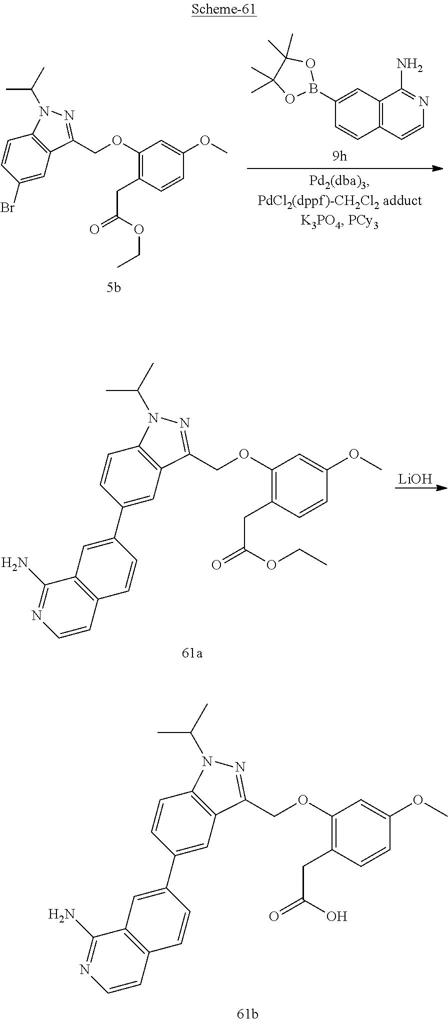

### Preparation of 2-(2-((5-(1-aminoisoquinolin-7-yl)-1-isopropyl-1H-indazol-3-yl)methoxy)-4-methoxyphenyl)acetic acid (61b)

### Step-1: Preparation of ethyl 2-(2-((5-(1-aminoisoquinolin-7-yl)-1-isopropyl-1H-indazol-3-yl)methoxy)-4-methoxyphenyl)acetate (61a)

Compound 61a was prepared according to the procedure reported in step-5 of scheme-1, from ethyl 2-(2-((5-bromo-1-isopropyl-1H-indazol-3-yl)methoxy)-4-methoxyphenyl)acetate (5b) (180 mg, 0.390 mmol) in dioxane/THF (4 mL, 1:1) using 7-(4,4,5,5-tetramethyl-1,3,2-dioxaborolan-2-yl)isoquinolin-1-amine (9h) (158 mg, 0.585 mmol), K₃PO₄ (2M aqueous solution, 0.780 mL, 1.561 mmol), PCy₃ (21.88 mg, 0.078 mmol), Pd₂(dba)₃ (35.7 mg, 0.039 mmol) and Pd(dppf)Cl₂—CH₂Cl₂ adduct (31.9 mg, 0.039 mmol) to afford after workup and purification using method-Y, ethyl 2-(2-((5-(1-aminoisoquinolin-7-yl)-1-isopropyl-1H-indazol-3-yl)methoxy)-4-methoxyphenyl)acetate (61a) (150 mg, 73.3% yield) as a clear oil; 1H NMR (300 MHz, DMSO-d₆) δ 8.52 (s, 1H), 8.14 (s, 1H), 8.06-7.90 (m, 2H), 7.90-7.72 (m, 3H), 7.55 (s, 1H), 7.07 (d, J=8.3 Hz, 1H), 6.97-6.84 (m, 3H), 6.48 (d, J=8.1 Hz, 1H), 5.45 (s, 2H), 5.13-4.97 (m, 1H), 3.77 (s, 3H), 3.62 (m, 2H), 3.45 (s, 2H), 1.52 (d, J=6.5 Hz, 6H), 0.72 (t, J=7.1 Hz, 3H); MS (ES+): 525.30 (M+1).

### Step-2: Preparation of 2-(2-((5-(1-aminoisoquinolin-7-yl)-1-isopropyl-1H-indazol-3-yl)methoxy)-4-methoxyphenyl)acetic acid (61b)

Compound 61b was prepared according to the procedure reported in step-6 of scheme-1, from ethyl 2-(2-((5-(1-aminoisoquinolin-7-yl)-1-isopropyl-1H-indazol-3-yl)methoxy)-4-methoxyphenyl)acetate (61a) (150 mg, 0.286 mmol) in THE/MeOH (4 mL, 1:1) using a solution of LiOH·H₂O (72.0 mg, 1.716 mmol) in water (I mL) to afford after workup and purification using method-G, 2-(2-((5-(1-aminoisoquinolin-7-yl)-1-isopropyl-1H-indazol-3-yl)methoxy)-4-methoxyphenyl)acetic acid (61b) (50 mg, 35.2% yield) HCl salt as a white solid; H NMR (300 MHz, DMSO-d₆) δ 13.45 (s, 1H, D₂O exchangeable), 12.07 (s, 1H, D₂O exchangeable), 9.25 (s, 2H, D₂O exchangeable), 8.98 (s, 1H), 8.41 (dd, J=8.5, 1.6 Hz, 1H), 8.31 (s, 1H), 8.10-7.96 (m, 2H), 7.91 (d, J=8.9 Hz, 1H), 7.69 (d, J=6.9 Hz, 1H), 7.27 (d, J=6.9 Hz, 1H), 7.08 (d, J=8.3 Hz, 1H), 6.90 (d, J=2.4 Hz, 1H), 6.48 (dd, J=8.3, 2.3 Hz, 1H), 5.49 (s, 2H), 5.15-5.00 (m, 1H), 3.76 (s, 3H), 3.45 (s, 2H), 1.53 (d, J=6.5 Hz, 6H); MS (ES+): 497.20 (M+1); (ES−): 495.20 (M−1).

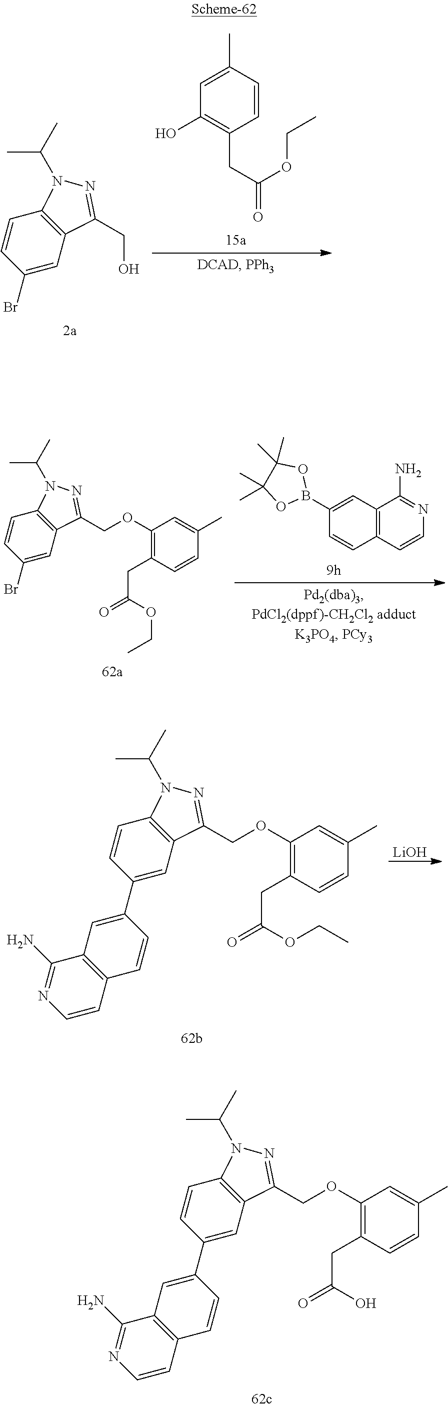

### Preparation of 2-(2-((5-(1-aminoisoquinolin-7-yl)-1-isopropyl-1H-indazol-3-yl)methoxy)-4-methylphenyl)acetic acid (62c)

### Step-1: Preparation of ethyl 2-(2-((5-bromo-1-isopropyl-1H-indazol-3-yl)methoxy)-4-methylphenyl)acetate (62a)

Compound 62a was prepared according to the procedure reported in step-2 of scheme-2, from (5-bromo-1-isopropyl-1H-indazol-3-yl)methanol (2a) (330 mg, 1.226 mmol) in DCM (10 mL), using ethyl 2-(2-hydroxy-4-methylphenyl)acetate (15a) (250 mg, 1.287 mmol) PPh₃ (386 mg, 1.471 mmol) and a solution of DCAD (540 mg, 1.471 mmol) in DCM (10 mL) to afford after workup and purification using method-N, ethyl 2-(2-((5-bromo-1-isopropyl-1H-indazol-3-yl)methoxy)-4-methylphenyl)acetate (62a) (400 mg, 73.3% yield) as a colorless oil; MS (ES+): 445.10 & 447.10 (M+1); (ES−): 443.10 & 445.00 (M−1).

### Step-2: Preparation of ethyl 2-(2-((5-(1-aminoisoquinolin-7-yl)-1-isopropyl-1H-indazol-3-yl)methoxy)-4-methylphenyl)acetate (62b)

Compound 62b was prepared according to the procedure reported in step-5 of scheme-1, from ethyl 2-(2-((5-bromo-1-isopropyl-1H-indazol-3-yl)methoxy)-4-methylphenyl)acetate (62a) (200 mg, 0.449 mmol) in dioxane/THF (4 mL, 1:1) using 7-(4,4,5,5-tetramethyl-1,3,2-dioxaborolan-2-yl)isoquinolin-1-amine (9h) (182 mg, 0.674 mmol). K₃PO₄ (2M aqueous solution, 0.898 mL, 1.796 mmol), PCy₃ (25.2 mg, 0.090 mmol), Pd₂(dba)₃ (41.1 mg, 0.045 mmol) and Pd(dppf)Cl₂—CH₂Cl₂ adduct (36.7 mg, 0.045 mmol) to afford after workup and purification using method-Y, ethyl 2-(2-((5-(1-aminoisoquinolin-7-yl)-1-isopropyl-1H-indazol-3-yl)methoxy)-4-methylphenyl)acetate (62b) (150 mg, 65.7% yield) as a clear oil; H NMR (300 MHz, DMSO-d₆) δ 8.53 (s, 1H), 8.14 (s, 1H), 8.05-7.89 (m, 2H), 7.88-7.72 (m, 3H), 7.15 (s, 1H), 7.04 (d, J=7.4 Hz, 1H), 6.98-6.86 (m, 3H), 6.72 (d, J=7.5 Hz, 1H), 5.44 (s, 2H), 5.12-4.98 (m, 1H), 3.63 (q, J=7.0 Hz, 2H), 3.47 (s, 2H), 2.31 (s, 3H), 1.53 (d, J=6.4 Hz, 6H), 0.73 (t, J=7.0 Hz, 3H); MS (ES+): 509.20 (M+1).

### Step-3: Preparation of 2-(2-((5-(1-aminoisoquinolin-7-yl)-1-isopropyl-1H-indazol-3-yl)methoxy)-4-methylphenyl)acetic acid (62c)

Compound 62c was prepared according to the procedure reported in step-6 of scheme-1, from ethyl 2-(2-((5-(1-aminoisoquinolin-7-yl)-1-isopropyl-1H-indazol-3-yl)methoxy)-4-methylphenyl)acetate (62b) (150 mg, 0.295 mmol) in THE/MeOH (4 mL, 1:1) using a solution of LiOH·H₂O (74.3 mg, 1.770 mmol) in water (1 mL) to afford after workup and purification using method-G, 2-(2-((5-(1-aminoisoquinolin-7-yl)-1-isopropyl-1H-indazol-3-yl)methoxy)-4-methylphenyl)acetic acid (62c) (55 mg, 38.8% yield) HCl salt as a white solid; ¹H NMR (300 MHz, DMSO-d₆) δ 13.47 (s, 1H, D₂O) exchangeable), 12.09 (s, 1H, D₂O exchangeable), 9.25 (s, 2H, D₂O exchangeable), 8.98 (s, 1H), 8.41 (dd, J=8.5, 1.6 Hz, 1H), 8.30 (s, 1H), 8.08-7.95 (m, 2H), 7.90 (d, J=8.9 Hz, 1H), 7.69 (d, J=6.9 Hz, 1H), 7.27 (d, J=6.9 Hz, 1H), 7.15 (s, 1H), 7.05 (d, J=7.5 Hz, 1H), 6.72 (d, J=7.6 Hz, 1H), 5.47 (s, 2H), 5.15-4.99 (m, 1H), 3.47 (s, 2H), 2.30 (s, 3H), 1.53 (d, J=6.5 Hz, 6H); MS (ES+): 481.20 (M+1); (ES−): 479.20 (M−1).

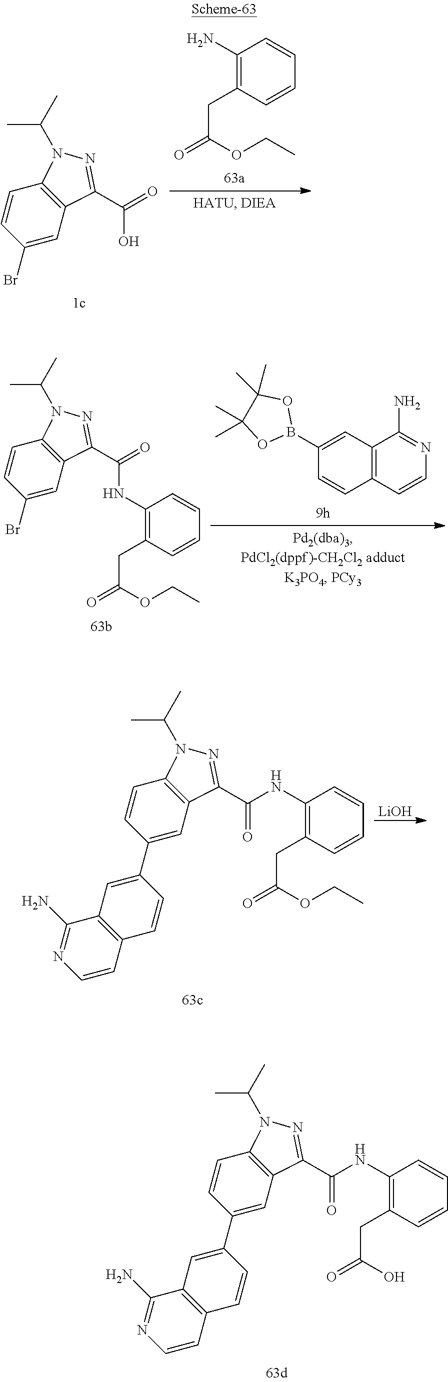

### Preparation of 2-(2-(5-(1-aminoisoquinolin-7-yl)-1-isopropyl-1H-indazole-3-carboxamido)phenyl)acetic acid (63d)

### Step-1: Preparation of ethyl 2-(2-(5-bromo-1-isopropyl-1H-indazole-3-carboxamido)phenyl)acetate (63b)

Compound 63b was prepared according to the procedure reported in step-3 of scheme-1, from 5-bromo-1-isopropyl-1H-indazole-3-carboxylic acid (1c) (400 mg, 1.413 mmol) in DMF (8 mL) using ethyl 2-(2-aminophenyl)acetate (63a) (266 mg, 1.483 mmol; CAS #64460-85-1) DIEA (0.984 mL, 5.65 mmol) HATU (806 mg, 2.119 mmol) to afford after workup and purification using method-X, ethyl 2-(2-(5-bromo-1-isopropyl-1H-indazole-3-carboxamido)phenyl)acetate (63b) (450 mg, 71.7% yield) as a white solid; ¹H NMR (300 MHz, DMSO-d₆) δ 9.87 (s, 1H), 8.32 (d, J=1.9 Hz, 1H), 7.88 (d, J=9.0 Hz, 1H), 7.68 (d, J=7.8 Hz, 1H), 7.61 (dd, J=9.0, 1.9 Hz, 1H), 7.34 (td, J=6.9, 6.2, 1.7 Hz, 2H), 7.28-7.17 (m, 1H), 5.15 (h, J=6.5 Hz, 1H), 4.06-3.98 (m, 2H), 3.79 (s, 2H), 1.56 (d, J=6.6 Hz, 6H), 1.05 (t, J=7.1 Hz, 3H); MS (ES+): 444.10 & 446.10 (M+1); MS (ES−): 442.00 & 444.00 (M−1).

### Step-2: Preparation of ethyl 2-(2-(5-(1-aminoisoquinolin-7-yl)-1-isopropyl-1H-indazole-3-carboxamido)phenyl)acetate (63c)

Compound 63c was prepared according to the procedure reported in step-5 of scheme-1, from ethyl 2-(2-(5-bromo-1-isopropyl-1H-indazole-3-carboxamido)phenyl)acetate (63b) (150 mg, 0.338 mmol) in dioxane/THF (4 mL, 1:1) using 7-(4,4,5,5-tetramethyl-1,3,2-dioxaborolan-2-yl)isoquinolin-1-amine (9h) (137 mg, 0.506 mmol), K₃PO₄ (2M aqueous solution, 0.675 mL, 1.350 mmol), PCy₃ (18.93 mg, 0.068 mmol), Pd₂(dba)₃ (30.9 mg, 0.034 mmol) and Pd(dppf)Cl₂—CH₂Cl₂ adduct (27.6 mg, 0.034 mmol) to afford after workup and purification using method-Y, ethyl 2-(2-(5-(1-aminoisoquinolin-7-yl)-1-isopropyl-1H-indazole-3-carboxamido)phenyl)acetate (63c) (130 mg, 76% yield) as a clear oil; 1H NMR (300 MHz, DMSO-d₆) δ 9.86 (s, 1H), 8.57 (d, J=6.6 Hz, 2H), 8.00 (d, J=7.3 Hz, 3H), 7.86-7.72 (m, 3H), 7.37 (d, J=7.5 Hz, 2H), 7.20 (t, J=7.6 Hz, 1H), 7.05 (s, 2H), 6.95 (d, J=5.8 Hz, 1H), 5.32-5.08 (m, 1H), 4.06 (q. J=7.1 Hz, 2H), 3.95 (s, 2H), 1.61 (d, J=6.5 Hz, 6H), 1.13-1.08 (m, 3H); MS (ES+): 508.20 (M+1); (ES−): 506.20 (M−1).

### Step-3: Preparation of 2-(2-(5-(1-aminoisoquinolin-7-yl)-1-isopropyl-1H-indazole-3-carboxamido)phenyl)acetic acid (63d)

Compound 63d was prepared according to the procedure reported in step-6 of scheme-1, from ethyl 2-(2-(5-(1-aminoisoquinolin-7-yl)-1-isopropyl-1H-indazole-3-carboxamido)phenyl)acetate (63c) (130 mg, 0.256 mmol) in THE/MeOH (4 mL, 1:1) using a solution of LiOH·H₂O (64.5 mg, 1.537 mmol) in water (1 mL) to afford after workup and purification using method-G, 2-(2-(5-(1-aminoisoquinolin-7-yl)-1-isopropyl-1H-indazole-3-carboxamido)phenyl)acetic acid (63d) (40 mg, 32.6% yield) HCl salt as a white solid; 3H NMR (300 MHz, DMSO-d₆) δ 12.97 (s, 1H, D₂O exchangeable), 10.06 (s, 1H, D₂O exchangeable), 9.26 (s, 2H, D₂O exchangeable), 8.99 (s, 1H), 8.64 (s, 1H), 8.37 (dd, J=8.5, 1.6 Hz, 1H), 8.15-7.96 (m, 3H), 7.93-7.81 (m, 1H), 7.71 (d, J=6.9 Hz, 1H), 7.42-7.31 (m, 2H), 7.28 (d, J=6.9 Hz, 1H), 7.24-7.10 (m, 1H), 5.28-5.12 (m, 1H), 3.73 (s, 2H), 1.61 (d, J=6.5 Hz, 6H); MS (ES+): 480.20 (M+1); (ES−): 478.15 (M−1).

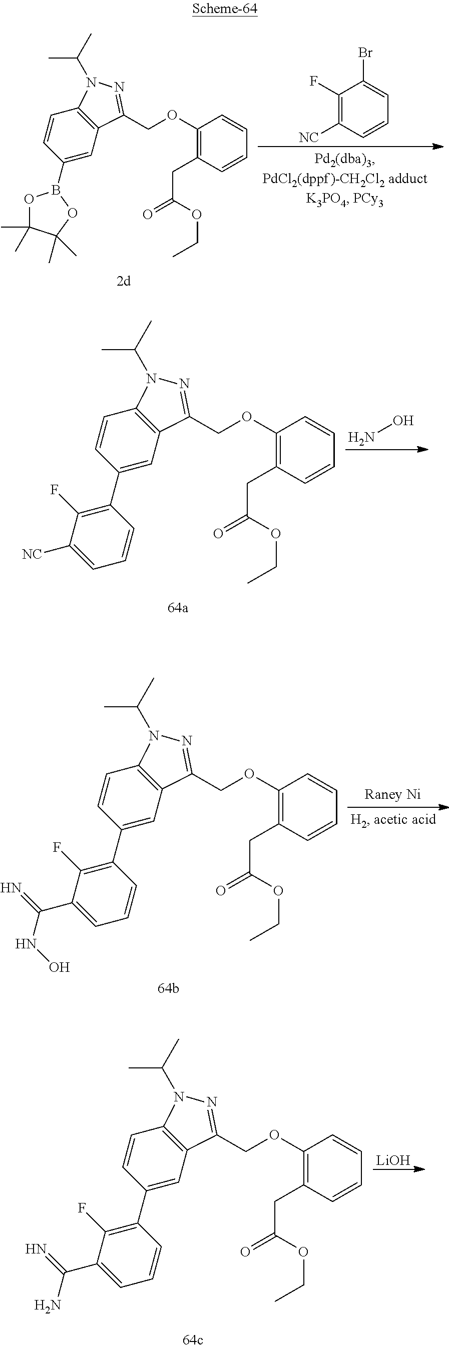

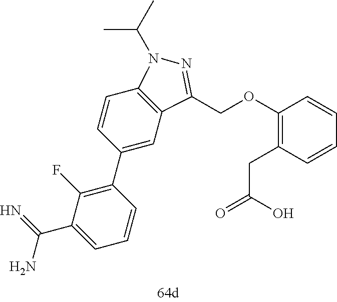 [SMILES: CC(C)n1nc(COc2ccccc2CC(=O)O)c2cc(-c3cccc(C(=N)N)c3F)ccc21]

### Preparation of 2-(2-((5-(3-carbamimidoyl-2-fluorophenyl)-1-isopropyl-1H-indazol-3-yl)methoxy)phenyl)acetic acid (64d)

### Step-1: Preparation of ethyl 2-(2-((5-(3-cyano-2-fluorophenyl)-1-isopropyl-1H-indazol-3-yl)methoxy)phenyl)acetate (64a)

Compound 64a was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (600 mg, 1.254 mmol) in dioxane/THF (6 mL, 1:1) using 3-bromo-2-fluorobenzonitrile (326 mg, 1.630 mmol; CAS #: 840481-82-5), K₃PO₄ (2M aqueous solution, 2.508 mL, 5.02 mmol), PCy₃ (70.3 mg, 0.251 mmol), PdCl₂(dppf)-CH₂Cl₂Adduct (102 mg, 0.125 mmol), and Pd₂(dba)₃ (115 mg, 0.125 mmol) to afford after workup and purification using method-P, ethyl 2-(2-((5-(3-cyano-2-fluorophenyl)-1-isopropyl-1H-indazol-3-yl)methoxy)phenyl)acetate (64a) (390 mg, 65.9% yield) as a clear oil; 1H NMR (300 MHz, DMSO-d₆) δ 8.02-7.89 (m, 3H), 7.89-7.82 (m, 1H), 7.67-7.58 (m, 1H), 7.51 (t, J=7.8 Hz, 1H), 7.29-7.25 (m, 2H), 7.21-7.14 (m, 1H), 6.96-6.84 (m, 1H), 5.43 (s, 2H), 5.06 (p, J=6.6 Hz, 1H), 3.65 (q, J=7.1 Hz, 2H), 3.53 (s, 2H), 1.52 (d, J=6.6 Hz, 6H), 0.81 (t, J=7.1 Hz, 3H); MS (ES+): 472.20 (M+1); 494.10 (M+Na).

### Step-2: Preparation of ethyl 2-(2-((5-(2-fluoro-3-(N-hydroxycarbamimidoyl)phenyl)-1-isopropyl-1H-indazol-3-yl)methoxy)phenyl)acetate (64b)

Compound 64b was prepared according to the procedure reported in step-5 of scheme-23 from ethyl 2-(2-((5-(3-cyano-2-fluorophenyl)-1-isopropyl-1H-indazol-3-yl)methoxy)phenyl)acetate (64a) (390 mg, 0.827 mmol) in EtOH (10 mL) using hydroxylamine (0.546 mL, 8.27 mmol) to afford after workup and purification using method-E, ethyl 2-(2-((5-(2-fluoro-3-(N-hydroxycarbamimidoyl)phenyl)-1-isopropyl-1H-indazol-3-yl)methoxy)phenyl)acetate (64b) (380 mg, 91% yield) as a white solid; MS (ES+): 505.20 (M+1); 527.20 (M+Na).

### Step-3: Preparation of ethyl 2-(2-((5-(3-carbamimidoyl-2-fluorophenyl)-1-isopropyl-1H-indazol-3-yl)methoxy)phenyl)acetate (64c)

Compound 64c was prepared according to the procedure reported in step-1 of scheme-24, from ethyl 2-(2-((5-(2-fluoro-3-(N-hydroxycarbamimidoyl)phenyl)-1-isopropyl-1H-indazol-3-yl)methoxy)phenyl)acetate (64b) (380 mg, 0.753 mmol) in EtOH (10 mL) using AcOH (0.043 mL, 0.753 mmol) and Raney Nickel (0.753 mmol) to afford after workup and purification using method-E, ethyl 2-(2-((5-(3-carbamimidoyl-2-fluorophenyl)-1-isopropyl-1H-indazol-3-yl)methoxy)phenyl)acetate (64c) (300 mg, 82% yield) as a colorless oil; MS (ES+): 489.25 (M+1).

### Step-4: Preparation of 2-(2-((5-(3-carbamimidoyl-2-fluorophenyl)-1-isopropyl-1H-indazol-3-yl)methoxy)phenyl)acetic acid (64d)

Compound 64d was prepared according to the procedure reported in step-6 of scheme-1 from ethyl 2-(2-((5-(3-carbamimidoyl-2-fluorophenyl)-1-isopropyl-1H-indazol-3-yl)methoxy)phenyl)acetate (64c) (300 mg, 0.614 mmol) in ACN/THF (6 mL, ratio: 1:1) using a solution of LiOH·H₂O (155 mg, 3.68 mmol) in water (2 mL) to afford after workup and purification using method-G, 2-(2-((5-(3-carbamimidoyl-2-fluorophenyl)-1-isopropyl-1H-indazol-3-yl)methoxy)phenyl)acetic acid (64d) (100 mg, 35.4% yield) HCl salt as a white solid; ¹H NMR (300 MHz, DMSO-d₆) δ 9.59 (s, 2H, D₂O exchangeable), 9.48 (s, 2H, D₂O exchangeable), 8.01 (s, 1H), 7.96-7.81 (m, 2H), 7.72-7.57 (m, 2H), 7.50 (t, J=7.7 Hz, 1H), 7.31-7.15 (m, 3H), 6.96-6.86 (m, 1H), 5.45 (s, 2H), 5.14-4.96 (m, 1H), 3.52 (s, 2H), 1.52 (d, J=6.6 Hz, 6H); ¹⁹F NMR (282 MHz, DMSO-d₆) δ −118.90 (m); MS (ES+): 461.20 (M+1); (ES−): 459.10 (M−1).

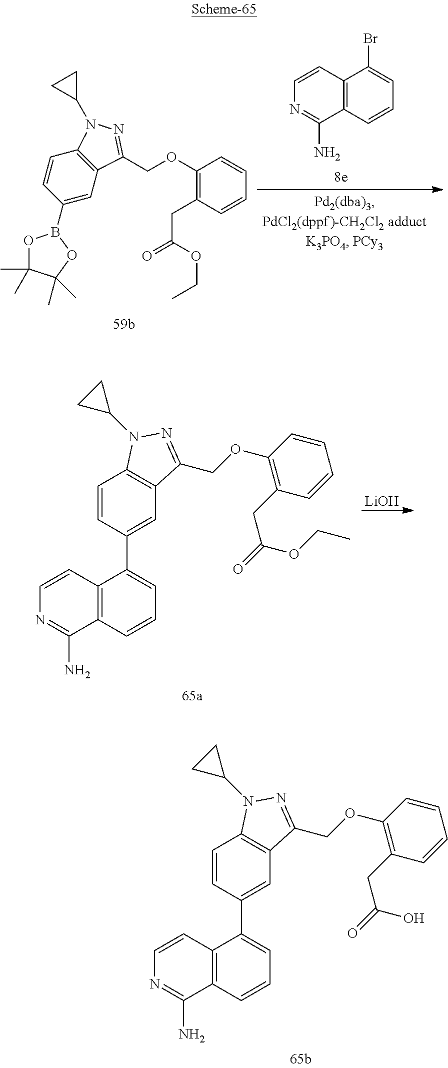

### Preparation of 2-(2-((5-(1-aminoisoquinolin-5-yl)-1-cyclopropyl-1H-indazol-3-yl)methoxy)phenyl)acetic acid (65b)

### Step-1: Preparation of ethyl 2-(2-((5-(1-aminoisoquinolin-5-yl)-1-cyclopropyl-1H-indazol-3-yl)methoxy)phenyl)acetate (65a)

Compound 65a was prepared according to the procedure reported in step-5 of scheme-1, from ethyl 2-(2-((1-cyclopropyl-5-(4,4,5,5-tetramethyl-1,3,2-dioxaborolan-2-yl)-1H-indazol-3-yl)methoxy)phenyl)acetate (59b) (200 mg, 0.420 mmol) in dioxane/THF (4 mL, 1:1) using 5-bromoisoquinolin-1-amine (8e) (112 mg, 0.504 mmol), K₃PO₄ (2M aqueous solution, 0.84 mL, 1.679 mmol), PCy₃ (23.55 mg, 0.084 mmol), Pd₂(dba)₃ (38.4 mg, 0.042 mmol) and Pd(dppf)Cl₂—CH₂Cl₂ adduct (34.3 mg, 0.042 mmol) to afford after workup and purification using method-E, ethyl 2-(2-((5-(1-aminoisoquinolin-5-yl)-1-cyclopropyl-1H-indazol-3-yl)methoxy)phenyl)acetate (65a) (150 mg, 72.5% yield) as a clear oil; ¹H NMR (300 MHz, DMSO-d₆) δ 8.22 (d, J=8.0 Hz, 1H), 7.86-7.77 (m, 2H), 7.75 (d, J=6.1 Hz, 1H), 7.59 (dd, J=7.2, 1.4 Hz, 1H), 7.56-7.48 (m, 2H), 7.25 (dd, J=6.1, 1.7 Hz, 2H), 7.19-7.11 (m, 1H), 6.94-6.85 (m, 3H), 6.80 (d, J=6.1 Hz, 1H), 5.39 (s, 2H), 3.88-3.76 (m, 1H), 3.54 (q, J=7.1 Hz, 2H), 3.49 (s, 2H), 1.21-1.12 (m, 4H), 0.71 (t, J=7.1 Hz, 3H); MS (ES+): 493.20 (M+1).

### Step-2: Preparation of 2-(2-((5-(1-aminoisoquinolin-5-yl)-1-cyclopropyl-1H-indazol-3-yl)methoxy)phenyl)acetic acid (65b)

Compound 65b was prepared according to the procedure reported in step-6 of scheme-1, from ethyl 2-(2-((5-(1-aminoisoquinolin-5-yl)-1-cyclopropyl-1H-indazol-3-yl)methoxy)phenyl)acetate (65a) (150 mg, 0.305 mmol) in THE/MeOH (4 mL, 1:1) using a solution of LiOH·H₂O (77 mg, 1.827 mmol) in water (1 mL) to afford after workup and purification using method-G, 2-(2-((5-(1-aminoisoquinolin-5-yl)-1-cyclopropyl-1H-indazol-3-yl)methoxy)phenyl)acetic acid (65b) (65 mg, 46.0% yield) HCl salt as a white solid; ¹H NMR (300 MHz, DMSO-d₆) δ 13.31 (s, 1H, D₂O exchangeable), 12.03 (s, 1H, D₂O exchangeable), 9.20 (s, 2H, D₂O exchangeable), 8.63 (d, J=8.3 Hz, 1H), 7.95 (d, J=7.2 Hz, 1H), 7.91-7.79 (m, 3H), 7.63 (d, J=7.2 Hz, 1H), 7.51 (dd, J=8.6, 1.7 Hz, 1H), 7.30-7.20 (m, 2H), 7.20-7.13 (m, 1H), 6.97 (d, J=7.2 Hz, 1H), 6.94-6.84 (m, 1H), 5.41 (s, 2H), 3.89-3.78 (m, 1H), 3.47 (s, 2H), 1.25-1.09 (m, 4H); MS (ES+): 465.20 (M+1); (ES−): 463.10 (M−1).

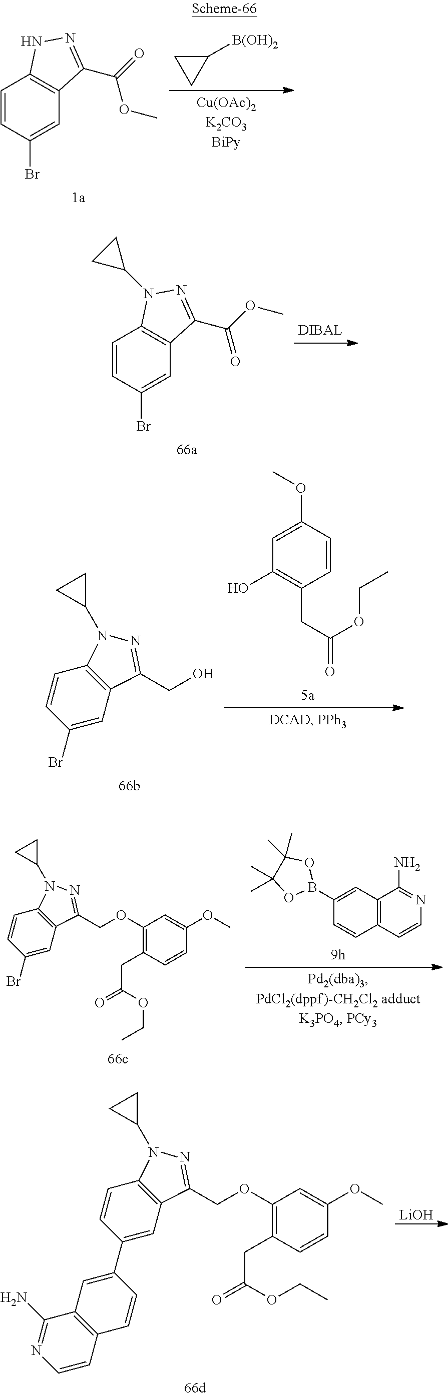

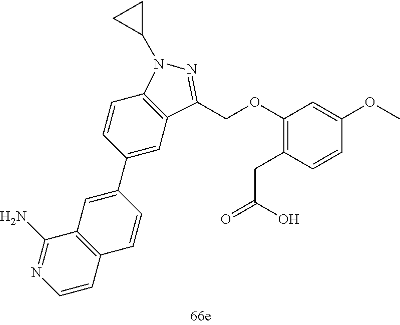 [SMILES: COc1ccc(CC(=O)O)c(OCc2nn(C3CC3)c3ccc(-c4ccc5ccnc(N)c5c4)cc23)c1]

### Preparation of 2-(2-((5-(1-aminoisoquinolin-7-yl)-1-cyclopropyl-1H-indazol-3-yl)methoxy)-4-methoxyphenyl)acetic acid (66e)

### Step-1: Preparation of methyl 5-bromo-1-cyclopropyl-1H-indazole-3-carboxylate (66a)

Compound 66a was prepared according to the procedure reported in step-1 of scheme-28, from methyl 5-bromo-1H-indazole-3-carboxylate (1a) (1 g, 3.92 mmol) in DCE (20 mL) using cyclopropyl boronic acid (0.674 g, 7.84 mmol), 2,2′-bipyridine (0.612 g, 3.92 mmol), Cu(OAc)₂ (0.712 g, 3.92 mmol) and K₂CO₃ (1.084 g, 7.84 mmol) to afford after workup and purification using method-AC, methyl 5-bromo-1-cyclopropyl-1H-indazole-3-carboxylate (66a) (420 mg, 136.3% yield) as a clear oil; MS (ES+): 295.00 & 297.00 (M+1); 317.00 & 319.00 (M+Na).

### Step-2: Preparation of (5-bromo-1-cyclopropyl-1H-indazol-3-yl)methanol (66b)

Compound 66b was prepared according to the procedure reported in step-1 of scheme-2, from methyl 5-bromo-1-cyclopropyl-1H-indazole-3-carboxylate (66a) (500 mg, 1.694 mmol) in DCM (20 mL) using DIBAL (1M solution in DCM, 4.24 mL, 4.24 mmol) to afford after workup and purification using method-J, (5-bromo-1-cyclopropyl-1H-indazol-3-yl)methanol (66b) (320 mg, 70.7% yield) as a light yellow oil; 1H NMR (300 MHz, DMSO-d₆) δ 8.05 (dd, J=1.9, 0.7 Hz, 1H), 7.63 (dd, J=8.9, 0.7 Hz, 1H), 7.52 (dd, J=8.8, 1.9 Hz, 1H), 5.33 (t, J=5.9 Hz, 1H), 4.71 (d, J=5.9 Hz, 2H), 3.75-3.64 (m, 1H), 1.12-1.03 (m, 4H); MS (ES+): 267.00 & 269.00 (M+1).

### Step-3: Preparation of ethyl 2-(2-((5-bromo-1-cyclopropyl-1H-indazol-3-yl)methoxy)-4-methoxyphenyl)acetate (66c)

Compound 66c was prepared according to the procedure reported in step-2 of scheme-2, from (5-bromo-1-cyclopropyl-1H-indazol-3-yl)methanol (66b) (320 mg, 1.198 mmol) in DCM (20 mL) using ethyl 2-(2-hydroxy-4-methoxyphenyl)acetate (5a) (277 mg, 1.318 mmol), PPh₃ (377 mg, 1.438 mmol) a solution of DCAD (528 mg, 1.438 mmol) in DCM (10 mL) to afford after workup and purification using method-P, ethyl 2-(2-((5-bromo-1-cyclopropyl-1H-indazol-3-yl)methoxy)-4-methoxyphenyl)acetate (66c) (280 mg, 50.9% yield) as a white oil; 1H NMR (300 MHz, DMSO-d₆) δ 7.95 (dd, J=1.8, 0.7 Hz, 1H), 7.69 (dd, J=8.9, 0.7 Hz, 1H), 7.57 (dd, J=8.9, 1.8 Hz, 1H), 7.08 (d, J=8.3 Hz, 1H), 6.82 (d, J=2.4 Hz, 1H), 6.49 (dd, J=8.3, 2.4 Hz, 1H), 5.34 (s, 2H), 3.92 (q. J=7.1 Hz, 2H), 3.83-3.77 (m, 1H), 3.76 (s, 3H), 3.44 (s, 2H), 1.17-1.07 (m, 4H), 0.96 (t, J=7.1 Hz, 3H); MS (ES+): 459.10 & 461.10 (M+1);

### Step-4: Preparation of ethyl 2-(2-((5-(1-aminoisoquinolin-7-yl)-1-cyclopropyl-1H-indazol-3-yl)methoxy)-4-methoxyphenyl)acetate (66d)

Compound 66d was prepared according to the procedure reported in step-5 of scheme-1, from ethyl 2-(2-((5-bromo-1-cyclopropyl-1H-indazol-3-yl)methoxy)-4-methoxyphenyl)acetate (66c) (140 mg, 0.305 mmol) in dioxane/THF (4 mL, 1:1) using 7-(4,4,5,5-tetramethyl-1,3,2-dioxaborolan-2-yl)isoquinolin-1-amine (9h) (124 mg, 0.457 mmol), K₃PO₄ (2M aqueous solution, 0.610 mL, 1.219 mmol), PCy₃ (17.09 mg, 0.061 mmol), Pd₂(dba)₃ (27.9 mg, 0.03 mmol) and Pd(dppf)Cl₂—CH₂Cl₂ adduct (24.9 mg, 0.03 mmol) to afford after workup and purification using method-Y, ethyl 2-(2-((5-(1-aminoisoquinolin-7-yl)-1-cyclopropyl-1H-indazol-3-yl)methoxy)-4-methoxyphenyl)acetate (66d) (120 mg, 75% yield) as a clear oil; ¹H NMR (300 MHz, DMSO-d₆) δ 8.53 (s, 1H), 8.16 (s, 1H), 8.00 (m, 2H), 7.88-7.72 (m, 3H), 7.07 (d, J=8.3 Hz, 1H), 7.00-6.79 (m, 4H), 6.48 (d, J=8.1 Hz, 1H), 5.41 (s, 2H), 3.89-3.81 (m, 1H), 3.77 (s, 3H), 3.68-3.56 (m, 2H), 3.43 (s, 2H), 1.69-1.61 (m, 2H), 1.38-1.29 (m, 2H), 0.73 (t, J=7.0 Hz, 3H); MS (ES+): 523.30 (M+1); (ES−): 521.20 (M−1).

### Step-5: Preparation of 2-(2-((5-(1-aminoisoquinolin-7-yl)-1-cyclopropyl-1H-indazol-3-yl)methoxy)-4-methoxyphenyl)acetic acid (66e)

Compound 66e was prepared according to the procedure and method of purification reported in step-6 of scheme-1 from ethyl 2-(2-((5-(1-aminoisoquinolin-7-yl)-1-cyclopropyl-1H-indazol-3-yl)methoxy)-4-methoxyphenyl)acetate (66d) (120 mg, 0.230 mmol) in THE/MeOH (4 mL, 1:1) using a solution of LiOH·H₂O (57.8 mg, 1.378 mmol) in water (1 mL) to afford after workup and purification using method-G, 2-(2-((5-(1-aminoisoquinolin-7-yl)-1-cyclopropyl-1H-indazol-3-yl)methoxy)-4-methoxyphenyl)acetic acid (66e) (50 mg, 0.101 mmol, 44.0% yield) HCl salt as a white solid; 1H NMR (300 MHz, DMSO-d₆) δ 13.36 (s, 1H, D₂O exchangeable), 9.21 (s, 2H, D₂O exchangeable), 8.97 (s, 1H), 8.41 (dd, J=8.5, 1.6 Hz, 1H), 8.31 (s, 1H), 8.09-7.98 (m, 2H), 7.87 (d, J=8.8 Hz, 1H), 7.73-7.65 (m, 1H), 7.27 (d, J=6.9 Hz, 1H), 7.08 (d, J=8.2 Hz, 1H), 6.87 (d, J=2.4 Hz, 1H), 6.49 (dd, J=8.4, 2.3 Hz, 1H), 5.44 (s, 2H), 3.92-3.82 (m, 1H), 3.76 (s, 3H), 3.43 (s, 2H), 1.25-1.12 (m, 4H); MS (ES+): 495.20 (M+1); (ES−): 493.20 (M−1).

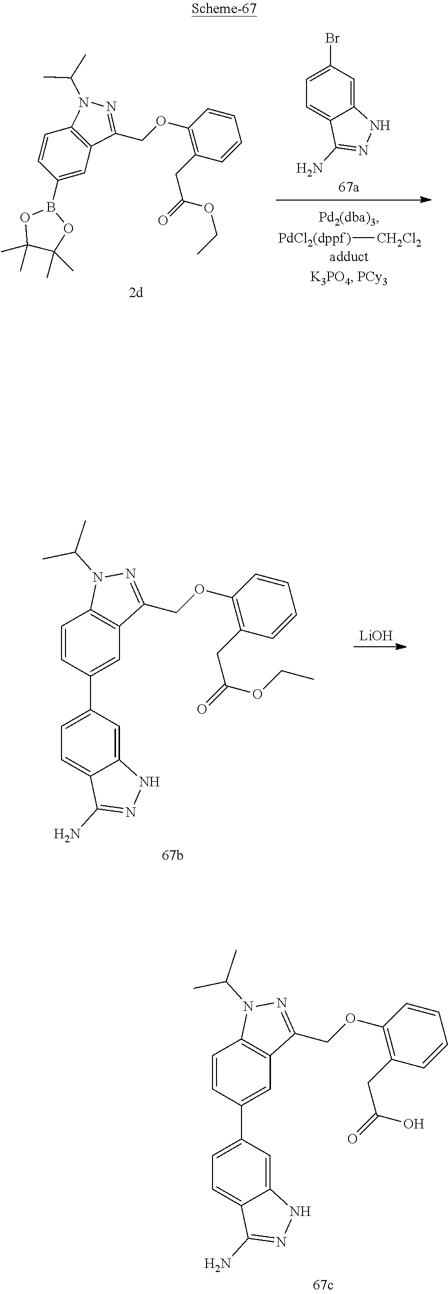

### Preparation of 2-(2-((3′-amino-1-isopropyl-1H, 1′H-[5,6′-biindazol]-3-yl)methoxy)phenyl)acetic acid (67c)

### Step-1: Preparation of ethyl 2-(2-((3′-amino-1-isopropyl-1H, 1′H-[5,6′-biindazol]-3-yl)methoxy)phenyl)acetate (67b)

Compound 67b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 6-bromo-1H-indazol-3-amine (67a) (100 mg, 0.470 mmol; CAS #404827-77-6), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.6 mg, 0.031 mmol) to afford after workup and purification using method-J, ethyl 2-(2-((3′-amino-1-isopropyl-1H, 1′H-[5,6′-biindazol]-3-yl)methoxy)phenyl)acetate (67b) (100 mg, 66.0% yield) as a clear oil; 1H NMR (300 MHz, DMSO-d₆) δ 11.41 (s, 1H), 7.98 (d, J=1.5 Hz, 1H), 7.83-7.70 (m, 3H), 7.45 (s, 1H), 7.28 (qd, J=8.6, 4.3 Hz, 3H), 7.19 (d, J=7.3 Hz, 1H), 6.96-6.88 (m, 1H), 5.44 (s, 2H), 5.37 (s, 2H), 5.03 (p. J=6.6 Hz, 1H), 3.63 (q, J=7.1 Hz, 2H), 3.52 (s, 2H), 1.52 (d, J=6.6 Hz, 6H), 0.74 (t, J=7.1 Hz, 3H); MS (ES+): 484.25 (M+1); (ES−): 482.10 (M−1).

### Step-2: Preparation of 2-(2-((3′-amino-1-isopropyl-1H, 1H-[5,6′-biindazol]-3-yl)methoxy)phenyl)acetic acid (67c)

Compound 67c was prepared according to the procedure reported in step-6 of scheme-1 from ethyl 2-(2-((3′-amino-1-isopropyl-1H, 1′H-[5,6′-biindazol]-3-yl)methoxy)phenyl)acetate (67b) (100 mg, 0.207 mmol) in THF/MeOH (4 mL, 1:1) using a solution of LiOH·H₂O (52.1 mg, 1.241 mmol) in water (1 mL) to afford after workup and purification using method-G, 2-(2-((3′-amino-1-isopropyl-1H, 1′H-[5,6′-biindazol]-3-yl)methoxy)phenyl)acetic acid (67c) (30 mg, 31.8% yield) HCl salt as a white solid; ¹H NMR (300 MHz, DMSO-d₆) δ 8.12 (s, 1H), 8.02 (d, J=8.5 Hz, 1H), 7.88-7.75 (m, 2H), 7.66 (s, 1H), 7.55 (dd, J=8.6, 1.4 Hz, 1H), 7.33-7.22 (m, 2H), 7.22-7.15 (m, 1H), 6.96-6.84 (m, 1H), 5.47 (s, 2H), 5.12-4.96 (m, 1H), 3.52 (s, 2H), 1.52 (d, J=6.6 Hz, 6H); MS (ES+): 456.20 (M+1); (ES−): 454.10 (M−1).

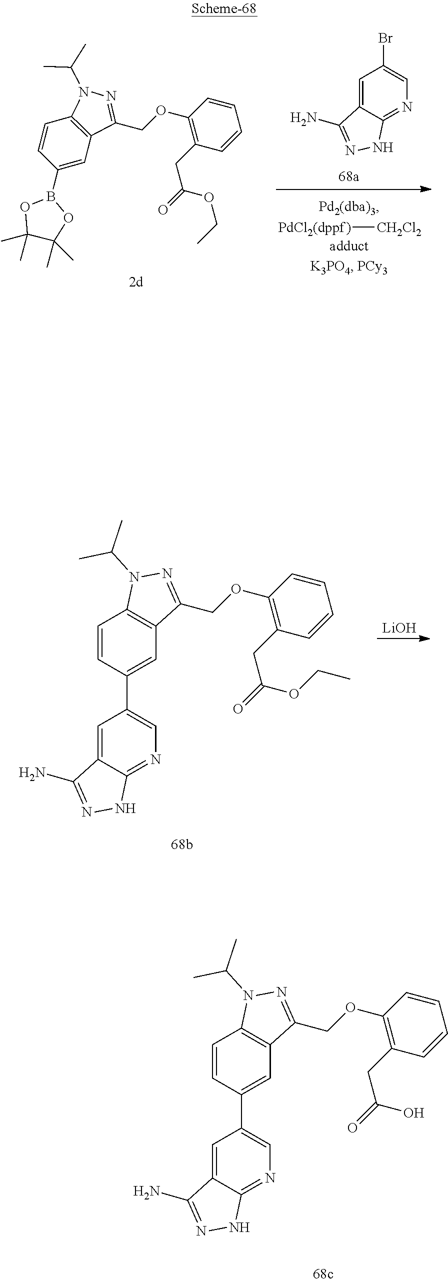

### Preparation of 2-(2-((5-(3-amino-1H-pyrazolo[3,4-b]pyridin-5-yl)-1-isopropyl-1H-indazol-3-yl)methoxy)phenyl)acetic acid (68c)

### Step-1: Preparation of ethyl 2-(2-((5-(3-amino-1H-pyrazolo[3,4-b]pyridin-5-yl)-1-isopropyl-1H-indazol-3-yl)methoxy)phenyl)acetate (68b)

Compound 68b was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, Ratio: 1:1) using 5-bromo-1H-pyrazolo[3,4-b]pyridin-3-amine (68a) (100 mg, 0.470 mmol; CAS #405224-24-0), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.6 mg, 0.031 mmol) to afford after workup and purification using method-J, ethyl 2-(2-((5-(3-amino-1H-pyrazolo[3,4-b]pyridin-5-yl)-1-isopropyl-1H-indazol-3-yl)methoxy)phenyl)acetate (68b) (100 mg, 65.8% yield) as a clear oil; ¹H NMR (300 MHz, DMSO-d₆) δ 11.99 (s, 1H), 8.71 (d, J=2.2 Hz, 1H), 8.41 (d, J=2.2 Hz, 1H), 8.03-7.96 (m, 1H), 7.85 (d, J=8.8 Hz, 1H), 7.72 (dd, J=8.9, 1.6 Hz, 1H), 7.34-7.25 (m, 2H), 7.19 (d, J=7.3 Hz, 1H), 6.91 (tt, J=8.9, 4.1 Hz, 1H), 5.62 (s, 2H), 5.43 (s, 2H), 5.05 (p. J=6.5 Hz, 1H), 3.62 (q, J=7.1 Hz, 2H), 3.53 (s, 2H), 1.52 (d, J=6.6 Hz, 6H), 0.73 (t, J=7.1 Hz, 3H); MS (ES+): 485.20 (M+1); (ES−): 483.20 (M−1).

### Step-2: Preparation of 2-(2-((5-(3-amino-1H-pyrazolo[3,4-b]pyridin-5-yl)-1-isopropyl-1H-indazol-3-yl)methoxy)phenyl)acetic acid (68c)

Compound 68c was prepared according to the procedure reported in step-6 of scheme-1 from ethyl 2-(2-((5-(3-amino-1H-pyrazolo[3,4-b]pyridin-5-yl)-1-isopropyl-1H-indazol-3-yl)methoxy)phenyl)acetate (68b) (100 mg, 0.206 mmol) in THF/MeOH (4 mL, 1:1) using a solution of LiOH·H₂O (52.0 mg, 1.238 mmol) in water (1 mL) to afford after workup and purification using method-G, 2-(2-((5-(3-amino-1H-pyrazolo[3,4-b]pyridin-5-yl)-1-isopropyl-1H-indazol-3-yl)methoxy)phenyl)acetic acid (68c) (20 mg, 0.044 mmol, 21.23% yield) HCl salt as a white solid; ¹H NMR (300 MHz, DMSO-d₆) δ 8.84 (d, J=2.1 Hz, 1H), 8.57 (s, 1H), 8.07 (s, 1H), 7.86 (d, J=8.8 Hz, 1H), 7.73 (dd, J=8.9, 1.7 Hz, 1H), 7.33-7.22 (m, 2H), 7.22-7.15 (m, 1H), 6.96-6.84 (m, 1H), 5.45 (s, 2H), 5.11-4.96 (m, 1H), 3.52 (s, 2H), 1.52 (d, J=6.5 Hz, 6H); MS (ES+): 457.20 (M+1); (ES−): 455.15 (M−1).

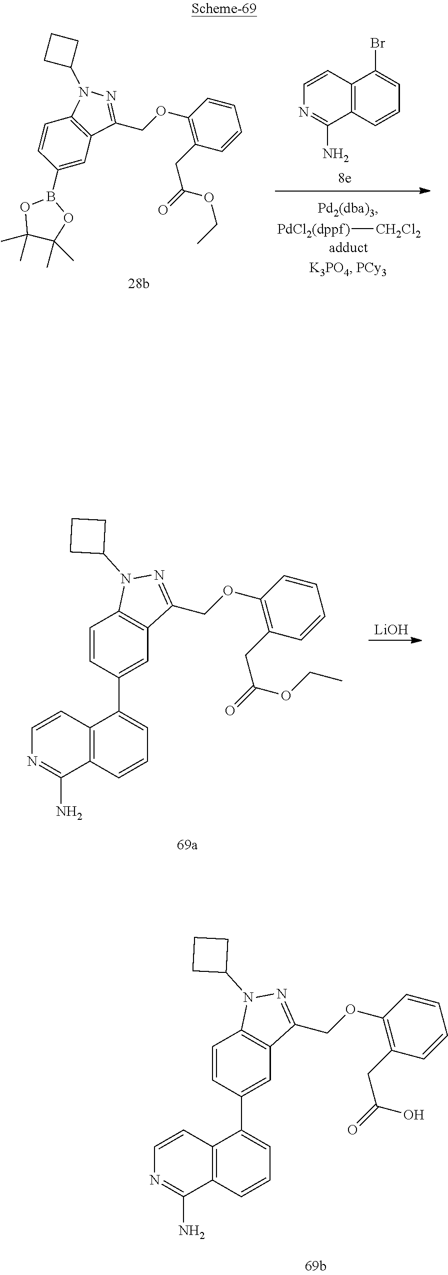

### Preparation of 2-(2-((5-(1-aminoisoquinolin-5-yl)-1-cyclobutyl-1H-indazol-3-yl)methoxy)phenyl)acetic acid (69b)

### Step-1: Preparation of ethyl 2-(2-((5-(1-aminoisoquinolin-5-yl)-1-cyclobutyl-1H-indazol-3-yl)methoxy)phenyl)acetate (69a)

Compound 69a was prepared according to the procedure reported in step-5 of scheme-1 from ethyl 2-(2-((1-cyclobutyl-5-(4,4,5,5-tetramethyl-1,3,2-dioxaborolan-2-yl)-1H-indazol-3-yl)methoxy)phenyl)acetate (28b) (210 mg, 0.428 mmol) in dioxane/THF (4 mL, 1:1) using 5-bromoisoquinolin-1-amine (5e) (105 mg, 0.471 mmol), K₃PO₄ (2M aqueous solution, 0.856 mL, 1.713 mmol), PCy₃ (24.02 mg, 0.086 mmol), Pd₂(dba)₃ (39.2 mg, 0.043 mmol) and Pd(dppf)Cl₂—CH₂Cl₂ adduct (35 mg, 0.043 mmol) to afford after workup and purification using method-E, ethyl 2-(2-((5-(1-aminoisoquinolin-5-yl)-1-cyclobutyl-1H-indazol-3-yl)methoxy)phenyl)acetate (69a) (150 mg, 69.1% yield) as a clear oil; ¹H NMR (300 MHz. DMSO-d₆) δ 8.21 (d, J=7.8 Hz, 1H), 7.84-7.72 (m, 3H), 7.60-7.46 (m, 3H), 7.26 (d, J=4.1 Hz, 2H), 7.16 (d, J=7.3 Hz, 1H), 6.93-6.87 (m, 1H), 6.86 (s, 2H), 6.80 (d, J=6.0 Hz, 1H), 5.45 (s, 2H), 5.35 (p. J=8.3 Hz, 1H), 3.57-3.53 (m, 2H), 3.50 (s, 2H), 2.77-2.60 (m, 2H), 1.97-1.89 (m, 2H), 1.80-1.72 (m, 2H), 0.70 (t, J=7.1 Hz, 3H); MS (ES+): 507.20 (M+1).

### Step-2: Preparation of 2-(2-((5-(1-aminoisoquinolin-5-yl)-1-cyclobutyl-1H-indazol-3-yl)methoxy)phenyl)acetic acid (69b)

Compound 69b was prepared according to the procedure reported in step-6 of scheme-1 from ethyl 2-(2-((5-(1-aminoisoquinolin-5-yl)-1-cyclobutyl-1H-indazol-3-yl)methoxy)phenyl)acetate (69a) (120 mg, 0.237 mmol) in THE/MeOH (4 mL, 1:1) using a solution of LiOH·H₂O (59.6 mg, 1.421 mmol) in water (I mL) to afford after workup and purification using method-G, 2-(2-((5-(1-aminoisoquinolin-5-yl)-1-cyclobutyl-1H-indazol-3-yl)methoxy)phenyl)acetic acid (69b) (95 mg, 0.199 mmol, 84% yield) HCl salt as a white solid; 1H NMR (300 MHz, DMSO-d₆) δ 13.36 (s, 1H, D₂O exchangeable), 12.04 (s, 1H, D₂O exchangeable), 9.22 (s, 2H, D₂O exchangeable), 8.63 (d, J=8.3 Hz, 1H), 7.94 (d, J=7.3 Hz, 1H), 7.91-7.79 (m, 3H), 7.62 (d, J=7.3 Hz, 1H), 7.50-7.40 (m, 1H), 7.30-7.21 (m, 2H), 7.17 (d, J=7.4 Hz, 1H), 6.97 (d, J=7.2 Hz, 1H), 6.93-6.83 (m, 1H), 5.47 (s, 2H), 5.44-5.25 (m, 1H), 3.48 (s, 2H), 2.79-2.58 (m, 2H), 2.58-2.51 (m, 2H), 2.00-1.82 (m, 2H); MS (ES+): 479.20 (M+1); (ES−): 477.15 (M−1); Analysis calculated for C₂₉H₂₆N₄O₃·HCl·1.75H₂O: C, 63.73; H, 5.63; Cl, 6.49; N, 10.25; Found: C, 63.65; H, 5.45; Cl, 6.82; N, 10.15.

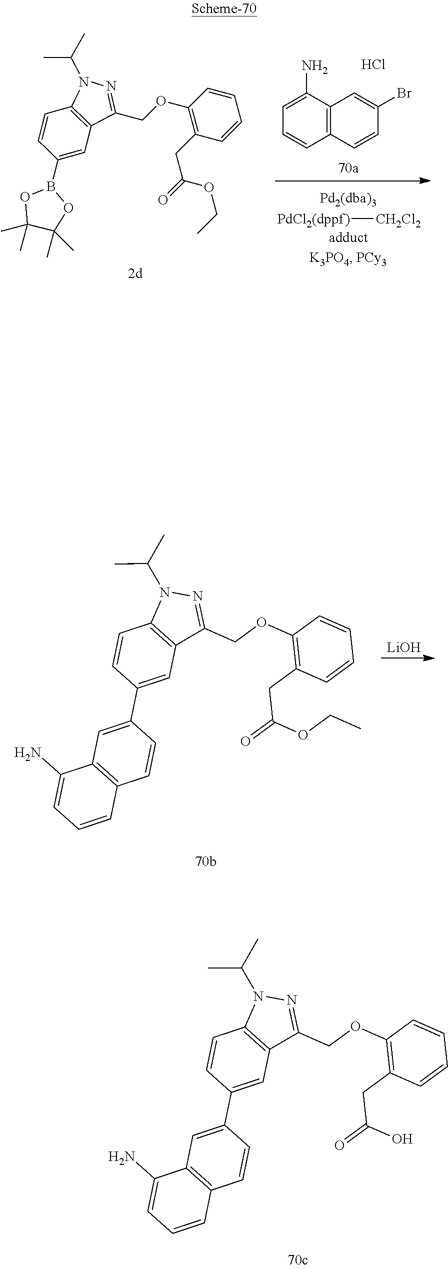

### Preparation of 2-(2-((5-(8-aminonaphthalen-2-yl)-1-isopropyl-1H-indazol-3-yl)methoxy)phenyl)acetic acid (70c)

### Step-1: Preparation of ethyl 2-(2-((5-(8-aminonaphthalen-2-yl)-1-isopropyl-1H-indazol-3-yl)methoxy)phenyl)acetate (70b)

Compound 70b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 7-bromonaphthalen-1-amine hydrochloride (70a) (97 mg, 0.376 mmol; CAS #137466-04-7), K₃PO₄ (2M aqueous solution, 0.627 mL, 1.254 mmol), PCy₃ (17.59 mg, 0.063 mmol), Pd₂(dba)₃ (28.7 mg, 0.031 mmol) and Pd(dppf)Cl₂—CH₂Cl₂ adduct (25.6 mg, 0.031 mmol) to afford after workup and purification using method-AC, ethyl 2-(2-((5-(8-aminonaphthalen-2-yl)-1-isopropyl-1H-indazol-3-yl)methoxy)phenyl)acetate (70b) (120 mg, 78% yield) as a clear oil; 1H NMR (300 MHz, DMSO-d₆) δ 8.38 (s, 1H), 8.13 (s, 1H), 7.95 (dd, J=8.9, 1.7 Hz, 1H), 7.84 (d, J=8.9 Hz, 1H), 7.80 (s, 2H), 7.36-7.25 (m, 2H), 7.19 (dt, J=7.5, 4.0 Hz, 2H), 7.09 (d, J=8.0 Hz, 1H), 6.97-6.86 (m, 1H), 6.68 (d, J=7.4 Hz, 1H), 5.83 (s, 2H), 5.45 (s, 2H), 5.06 (p. J=6.6 Hz, 1H), 3.65 (q, J=7.1 Hz, 2H), 3.54 (s, 2H), 1.53 (d, J=6.6 Hz, 6H), 0.72 (t, J=7.1 Hz, 3H); MS (ES+): 494.20 (M+1); (ES−): 492.15 (M−1).

### Step-2: Preparation of 2-(2-((5-(8-aminonaphthalen-2-yl)-1-isopropyl-1H-indazol-3-yl)methoxy)phenyl)acetic acid (70c)

Compound 70c was prepared according to the procedure reported in step-6 of scheme-1 from ethyl 2-(2-((5-(8-aminonaphthalen-2-yl)-1-isopropyl-1H-indazol-3-yl)methoxy)phenyl)acetate (70b) (120 mg, 0.243 mmol) in THF/MeOH (4 mL, 1:1) using a solution of LiOH·H₂O (61.2 mg, 1.459 mmol) in water (1 mL) to afford after workup and purification using method-G, 2-(2-((5-(8-aminonaphthalen-2-yl)-1-isopropyl-1H-indazol-3-yl)methoxy)phenyl)acetic acid (70c) (65 mg, 57.4% yield) HCl salt as a white solid; ¹H NMR (300 MHz, DMSO-d₆) δ 8.41 (s, 1H), 8.25 (s, 1H), 8.11-7.95 (m, 3H), 7.90 (d, J=8.9 Hz, 1H), 7.87-7.78 (m, 1H), 7.54-7.43 (m, 2H), 7.34-7.23 (m, 2H), 7.23-7.16 (m, 1H), 6.92 (td, 1H), 5.48 (s, 2H), 5.16-4.99 (m, 1H), 3.54 (s, 2H), 1.54 (d, J=6.5 Hz, 6H); MS (ES+): 466.20 (M+1); (ES−): 464.15 (M−1).

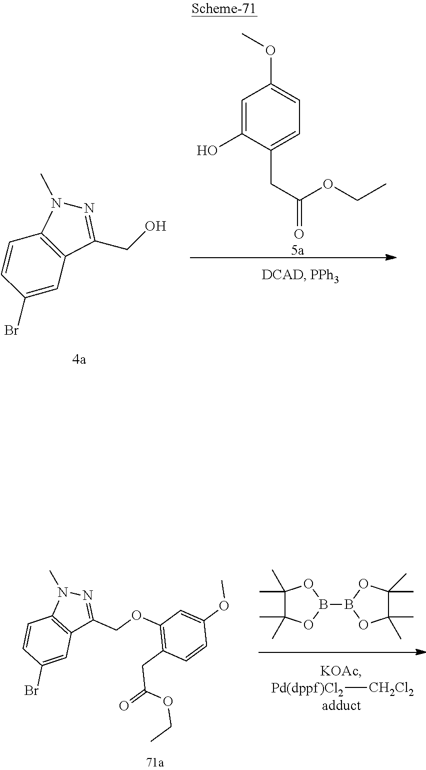

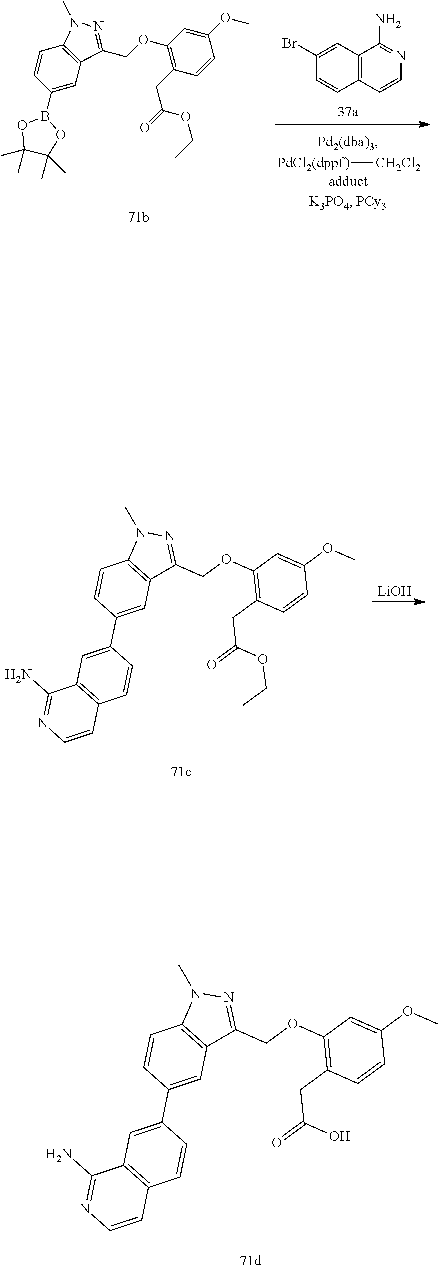

### Preparation of 2-(2-((5-(1-aminoisoquinolin-7-yl)-1-methyl-1H-indazol-3-yl)methoxy)-4-methoxyphenyl)acetic acid (71d)

### Step-1: Preparation of ethyl 2-(2-((5-bromo-1-methyl-1H-indazol-3-yl)methoxy)-4-methoxyphenyl)acetate (71a)

Compound 71a was prepared according to the procedure reported in step-2 of scheme-2 from (5-bromo-1-methyl-1H-indazol-3-yl)methanol (4a) (2.0 g, 8.30 mmol) in DCM (30 mL), using ethyl 2-(2-hydroxy-4-methoxyphenyl)acetate (5a) (1.918 g, 9.13 mmol), PPh₃ (2.61 g, 9.96 mmol) and a solution of DCAD (3.66 g, 9.96 mmol) in DCM (20 mL) to afford after workup and purification using method-H, ethyl 2-(2-((5-bromo-1-methyl-1H-indazol-3-yl)methoxy)-4-methoxyphenyl)acetate (71a) (2.4 g, 66.8% yield) as a white solid; ¹H NMR (300 MHz, DMSO-d₆) δ 7.95 (d, J=1.8 Hz, 1H), 7.66 (d, J=8.9 Hz, 1H), 7.54 (dd, J=8.9, 1.8 Hz, 1H), 7.08 (d, J=8.3 Hz, 1H), 6.83 (d, J=2.4 Hz, 1H), 6.49 (dd, J=8.3, 2.4 Hz, 1H), 5.35 (s, 2H), 5.08 (s, 2H), 4.05 (s, 3H), 3.91 (q. J=7.1 Hz, 2H), 3.76 (s, 3H), 0.95 (t, J=7.1 Hz, 3H); MS (ES+): 433.10 & 435.10 (M+1); (ES−): 431.00 & 433.00 (M−1).

### Step-2: Preparation of ethyl 2-(4-methoxy-2-((1-methyl-5-(4,4,5,5-tetramethyl-1,3,2-dioxaborolan-2-yl)-1H-indazol-3-yl)methoxy)phenyl)acetate (71b)

Compound 71b was prepared according to the procedure and method of purification reported in step-4 of scheme-1 from ethyl 2-(2-((5-bromo-1-methyl-1H-indazol-3-yl)methoxy)-4-methoxyphenyl)acetate (71a) (2 g, 4.62 mmol) in anhydrous dioxane (40 mL) using BISPIN (2.344 g, 9.23 mmol), KOAc (1.133 g, 11.54 mmol), PdCl₂(dppf)-CH₂Cl₂Adduct (0.226 g, 0.277 mmol) to afford after workup and purification using method-AF, ethyl 2-(4-methoxy-2-((1-methyl-5-(4,4,5,5-tetramethyl-1,3,2-dioxaborolan-2-yl)-1H-indazol-3-yl)methoxy)phenyl)acetate (71b) (1.7 g, 77% yield) as a clear oil; 1H NMR (300 MHZ, DMSO-d₆) δ 8.17 (t, J=1.0 Hz. JH), 7.72-7.57 (m, 2H), 7.08 (d, J=8.3 Hz, 1H), 6.88 (d, J=2.4 Hz, 1H), 6.49 (dd, J=8.3, 2.4 Hz, 1H), 5.38 (s, 2H), 4.05 (s, 3H), 3.85 (q, J=7.1 Hz, 2H), 3.77 (s, 3H), 3.41 (s, 2H), 1.30 (s, 12H), 0.89 (t, J=7.1 Hz, 3H); MS (ES+): 481.30 (M+1).

### Step-3: Preparation of ethyl 2-(2-((5-(1-aminoisoquinolin-7-yl)-1-methyl-1H-indazol-3-yl)methoxy)-4-methoxyphenyl)acetate (71c)

Compound 71c was prepared according to the procedure reported in step-5 of scheme-1 from ethyl 2-(4-methoxy-2-((1-methyl-5-(4,4,5,5-tetramethyl-1,3,2-dioxaborolan-2-yl)-1H-indazol-3-yl)methoxy)phenyl)acetate (71b) (200 mg, 0.416 mmol) in dioxane/THF (4 mL, 1:1) using 7-bromoisoquinolin-1-amine (37a) (93 mg, 0.416 mmol), K₃PO₄ (2M aqueous solution, 0.833 mL, 1.665 mmol), PCy₃ (23.35 mg, 0.083 mmol). Pd₂(dba)₃ (38.1 mg, 0.042 mmol), PdCl₂(dppf)-CH₂Cl₂Adduct (0.034 g, 0.042 mmol) to afford after workup and purification using method-AE, ethyl 2-(2-((5-(1-aminoisoquinolin-7-yl)-1-methyl-1H-indazol-3-yl)methoxy)-4-methoxyphenyl)acetate (71c) (150 mg, 0.302 mmol, 72.6% yield) as a clear oil; MS (ES+): 497.20 (M+1).

### Step-4: Preparation of 2-(2-((5-(1-aminoisoquinolin-7-yl)-1-methyl-1H-indazol-3-yl)methoxy)-4-methoxyphenyl)acetic acid (71d)

Compound 71d was prepared according to the procedure reported in step-6 of scheme-1 from ethyl 2-(2-((5-(1-aminoisoquinolin-7-yl)-1-methyl-1H-indazol-3-yl)methoxy)-4-methoxyphenyl)acetate (71c) (150 mg, 0.302 mmol) in MeOH/THF (6 mL, 1:1) using a solution of LiOH·H₂O (76 mg, 1.812 mmol) in water (1 mL) to afford after workup and purification using method-G, 2-(2-((5-(1-aminoisoquinolin-7-yl)-1-methyl-1H-indazol-3-yl)methoxy)-4-methoxyphenyl)acetic acid (71d) (80 mg, 56.5% yield) HCl salt as a white solid; 1H NMR (300 MHz, DMSO-d₆) δ 13.44 (s, 1H, D₂O exchangeable), 12.03 (s, 1H, D₂O exchangeable), 9.25 (s, 2H, D₂O exchangeable), 8.99 (s, 1H), 8.42 (dd, J=8.5, 1.6 Hz, 1H), 8.34 (s, 1H), 8.09-7.98 (m, 2H), 7.85 (d, J=8.8 Hz, 1H), 7.70 (d, J=6.9 Hz, 1H), 7.27 (d, J=6.9 Hz, 1H), 7.09 (d, J=8.3 Hz, 1H), 6.89 (d, J=2.4 Hz, 1H), 6.50 (dd, J=8.3, 2.3 Hz, 1H), 5.47 (s, 2H), 4.12 (s, 3H), 3.77 (s, 3H), 3.44 (s, 2H); MS (ES+): 469.20 (M+1); (ES−): 467.10 (M−1).

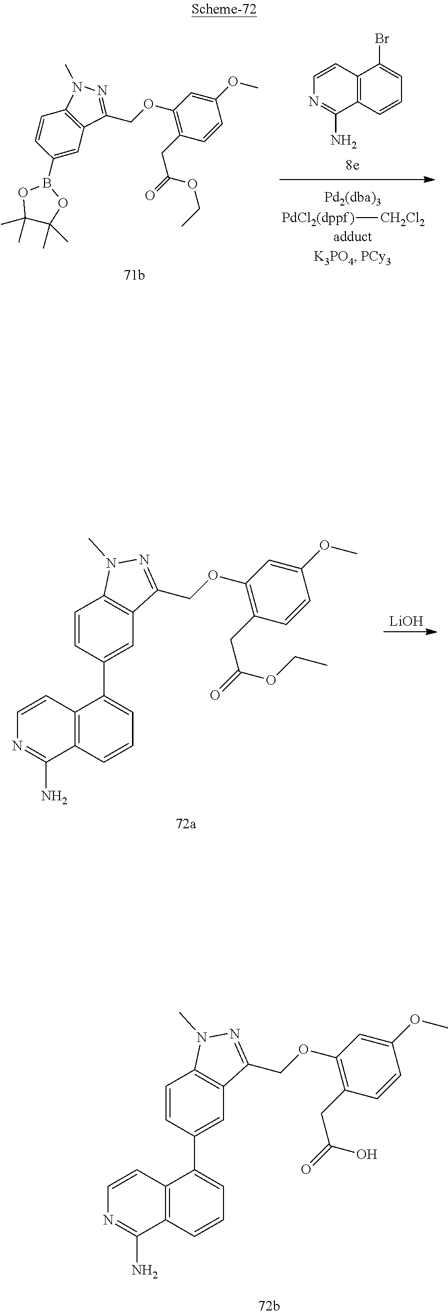

### Preparation of 2-(2-((5-(1-aminoisoquinolin-5-yl)-1-methyl-1H-indazol-3-yl)methoxy)-4-methoxyphenyl)acetic acid (72b)

### Step-1: Preparation of ethyl 2-(2-((5-(1-aminoisoquinolin-5-yl)-1-methyl-1H-indazol-3-yl)methoxy)-4-methoxyphenyl)acetate (72a)

Compound 72a was prepared according to the procedure reported in step-5 of scheme-1 from ethyl 2-(4-methoxy-2-((1-methyl-5-(4,4,5,5-tetramethyl-1,3,2-dioxaborolan-2-yl)-1H-indazol-3-yl)methoxy)phenyl)acetate (71b) (200 mg, 0.416 mmol) in dioxane/THF (4 mL, 1:1) using 5-bromoisoquinolin-1-amine (93 mg, 0.416 mmol), K₃PO₄ (2M aqueous solution, 0.833 mL, 1.665 mmol), PCy₃ (23.35 mg, 0.083 mmol), Pd₂(dba)₃ (38.1 mg, 0.042 mmol) and PdCl₂(dppf)-CH₂Cl₂Adduct (0.034 g, 0.042 mmol) to afford after workup and purification using method-E, ethyl 2-(2-((5-(1-aminoisoquinolin-5-yl)-1-methyl-1H-indazol-3-yl)methoxy)-4-methoxyphenyl)acetate (72a) (150 mg, 72.6% yield) as a clear oil; MS (ES+): 497.20 (M+1).

### Step-2: Preparation of 2-(2-((5-(1-aminoisoquinolin-5-yl)-1-methyl-1H-indazol-3-yl)methoxy)-4-methoxyphenyl)acetic acid (72b)

Compound 72b was prepared according to the procedure reported in step-6 of scheme-1 from ethyl 2-(2-((5-(1-aminoisoquinolin-5-yl)-1-methyl-1H-indazol-3-yl)methoxy)-4-methoxyphenyl)acetate (72a) (150 mg, 0.302 mmol) in MeOH/THF (6 mL, 1:1) using a solution of LiOH·H₂O (76 mg, 1.812 mmol) in water (2 mL) to afford after workup and purification using method-G, 2-(2-((5-(1-aminoisoquinolin-5-yl)-1-methyl-1H-indazol-3-yl)methoxy)-4-methoxyphenyl)acetic acid (72b) (75 mg, 53.0% yield) HCl salt as a white solid; 1H NMR (300 MHz, DMSO-d₆) δ 13.51 (s, 1H, D₂O exchangeable), 9.30 (s, 2H, D₂O exchangeable), 8.66 (d, J=8.3 Hz, 1H), 8.01-7.92 (m, 1H), 7.91-7.78 (m, 3H), 7.63 (d, J=7.3 Hz, 1H), 7.49 (dd, J=8.6, 1.6 Hz, 1H), 7.06 (d, J=8.3 Hz, 1H), 6.96 (d, J=7.2 Hz, 1H), 6.83 (d, J=2.4 Hz, 1H), 6.47 (dd, J=8.3, 2.3 Hz, 1H), 5.43 (s, 2H), 4.12 (s, 3H), 3.73 (s, 3H), 3.38 (s, 2H); MS (ES+): 469.10 (M+1); (ES−): 467.10 (M−1).

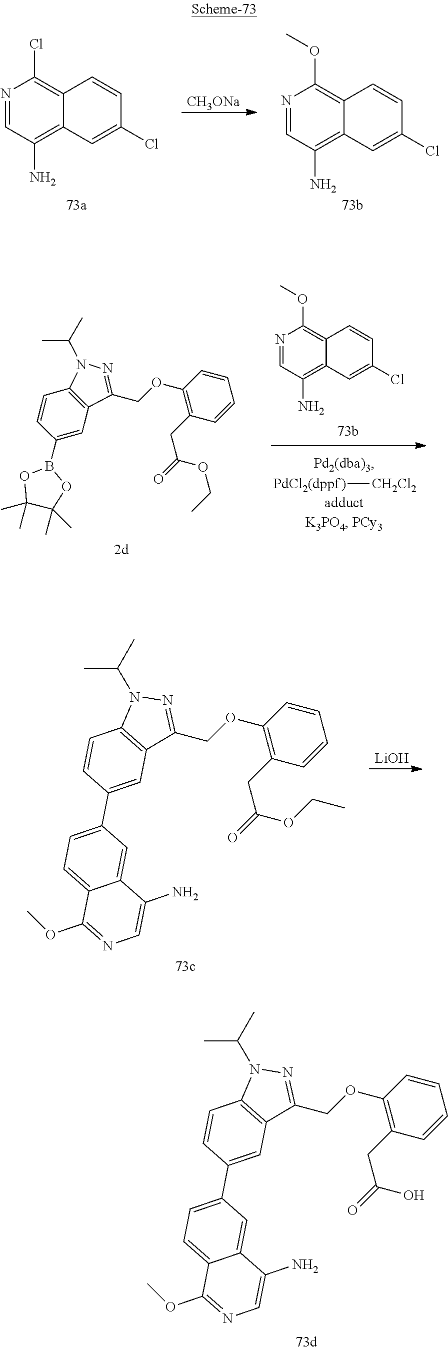

### Preparation of 2-(2-((5-(4-amino-1-methoxyisoquinolin-6-yl)-1-isopropyl-1H-indazol-3-yl)methoxy)phenyl)acetic acid (73d)

### Step-1: Preparation of 6-chloro-1-methoxyisoquinolin-4-amine (73b)

Compound 73b was prepared according to the procedure reported in step-1 of scheme-23 from 1,6-dichloroisoquinolin-4-amine (73a) (300 mg, 1.408 mmol) in MeOH (10 mL) using sodium methoxide (4.83 mL, 21.12 mmol) to afford after workup and purification using method-AG, 6-chloro-1-methoxyisoquinolin-4-amine (73b) (65 mg, 22.13% yield) as a white solid; 1H NMR (300 MHz, DMSO-d₆) δ 8.16 (d, J=2.1 Hz, 1H), 8.09 (d, J=8.8 Hz, 1H), 7.58 (dd, J=8.9, 2.1 Hz, 1H), 7.43 (s, 1H), 5.25 (s, 2H), 3.92 (s, 3H); MS (ES+): 209.00 & 211.00 (M+1).

### Step-2: Preparation of ethyl 2-(2-((5-(4-amino-1-methoxyisoquinolin-6-yl)-1-isopropyl-1H-indazol-3-yl)methoxy)phenyl)acetate (73c)

Compound 73c was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (140 mg, 0.293 mmol) in dioxane/THF (4 mL, 1:1) using 6-chloro-1-methoxyisoquinolin-4-amine (73b) (61.1 mg, 0.293 mmol). K₃PO₄ (2M aqueous solution, 0.585 mL, 1.171 mmol), PCy₃ (16.41 mg, 0.059 mmol), Pd₂(dba)₃ (26.8 mg, 0.029 mmol) and PdCl₂(dppf)-CH₂Cl₂ adduct (0.0239 g, 0.029 mmol) to afford after workup and purification using method-AC, ethyl 2-(2-((5-(4-amino-1-methoxyisoquinolin-6-yl)-1-isopropyl-1H-indazol-3-yl)methoxy)phenyl)acetate (73c) (120 mg, 78% yield) as a clear oil; MS (ES+): 525.20 (M+1).

### Step-3: Preparation of 2-(2-((5-(4-amino-1-methoxyisoquinolin-6-yl)-1-isopropyl-1H-indazol-3-yl)methoxy)phenyl)acetic acid (73d)

Compound 73d was prepared according to the procedure reported in step-6 of scheme-1 from ethyl 2-(2-((5-(4-amino-1-methoxyisoquinolin-6-yl)-1-isopropyl-1H-indazol-3-yl)methoxy)phenyl)acetate (73c) (120 mg, 0.229 mmol) in MeOH/THF (6 mL, 1:1) using a solution of LiOH·H₂O (57.6 mg, 1.372 mmol) in water (2 mL) to afford after workup and purification using method-G, 2-(2-((5-(4-amino-1-methoxyisoquinolin-6-yl)-1-isopropyl-1H-indazol-3-yl)methoxy)phenyl)acetic acid (73d) (25 mg, 22.01% yield) HCl salt as a white solid; 4H NMR (300 MHz, DMSO-d₆) δ 8.35-8.27 (m, 3H), 8.18-8.11 (m, 1H), 7.98-7.91 (m, 3H), 7.31-7.26 (m, 2H), 7.22 (d, J=7.0 Hz, 1H), 6.98-6.89 (m, 1H), 5.49 (s, 2H), 5.18-4.98 (m, 1H), 4.08 (s, 3H), 3.55 (s, 2H), 1.55 (d, J=6.5 Hz, 6H); MS (ES+): 497.20 (M+1); (ES−): 495.20 (M−1).

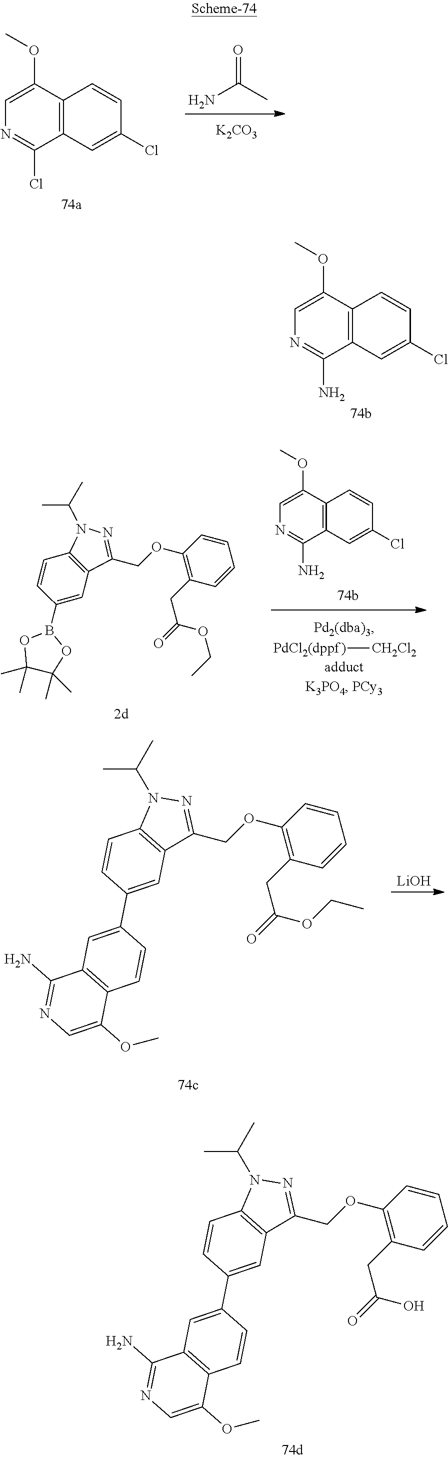

### Preparation of 2-(2-((5-(1-amino-4-methoxyisoquinolin-7-yl)-1-isopropyl-1H-indazol-3-yl)methoxy)phenyl)acetic acid (74d)

### Step-1: Preparation of 7-chloro-4-methoxyisoquinolin-1-amine (74b)

A mixture of 1,7-dichloro-4-methoxyisoquinoline (74a) (100 mg, 0.438 mmol; CAS #630423-36-8), acetamide (518 mg, 8.77 mmol) and K₂CO₃ (182 mg, 1.315 mmol) was heated in a microwave at 150° ° C. for 60 min. After cooling to room temperature, the mixture was dissolved in ethyl acetate, washed with water, brine, dried over MgSO₄ and concentrated. The residue was purified using method-AH, to give 7-chloro-4-methoxyisoquinolin-1-amine (74b) (30 mg, 32.8% yield) as a white solid; MS (ES+): 209.00 & 211.00 (M+1).

### Step-2: Preparation of ethyl 2-(2-((5-(1-amino-4-methoxyisoquinolin-7-yl)-1-isopropyl-1H-indazol-3-yl)methoxy)phenyl)acetate (74c)

Compound 74c was prepared according to the procedure reported in step-5 of scheme-1: to a degassed solution of ethyl 2-(2-((1-isopropyl-5-(4,4,5,5-tetramethyl-1,3,2-dioxaborolan-2-yl)-1H-indazol-3-yl)methoxy)phenyl)acetate (2d) (140 mg, 0.293 mmol) in dioxane/THF (4 mL, 1:1) using 7-chloro-4-methoxyisoquinolin-1-amine (74b) (61.1 mg, 0.293 mmol), K₃PO₄ (2M aqueous solution, 0.585 mL, 1.171 mmol), PCy₃ (16.41 mg, 0.059 mmol), PdCl₂(dppf)-CH₂Cl₂ adduct (0.0239 g, 0.029 mmol) and Pd₂(dba)₃ (26.8 mg, 0.029 mmol) to afford after workup and purification using method-AC, ethyl 2-(2-((5-(1-amino-4-methoxyisoquinolin-7-yl)-1-isopropyl-1H-indazol-3-yl)methoxy)phenyl)acetate (74c) (80 mg, 52.1% yield) as a clear oil; MS (ES+): 525.20 (M+1).

### Step-3: Preparation of 2-(2-((5-(1-amino-4-methoxyisoquinolin-7-yl)-1-isopropyl-1H-indazol-3-yl)methoxy)phenyl)acetic acid (74d)

Compound 74d was prepared according to the procedure reported in step-6 of scheme-1 from ethyl 2-(2-((5-(1-amino-4-methoxyisoquinolin-7-yl)-1-isopropyl-1H-indazol-3-yl)methoxy)phenyl)acetate (74c) (80 mg, 0.152 mmol) in MeOH/THF (6 mL, 1:1) using a solution of LiOH·H₂O (38.4 mg, 0.915 mmol) in water (2 mL) to afford after workup and purification using method-G, 2-(2-((5-(1-amino-4-methoxyisoquinolin-7-yl)-1-isopropyl-1H-indazol-3-yl)methoxy)phenyl)acetic acid (74d) (8 mg, 10.56% yield) HCl salt as a white solid; 1H NMR (300 MHz, DMSO-d₆) δ 13.18 (s, 1H, D₂O exchangeable), 12.13 (s, 1H, D₂O exchangeable), 8.95 (d, J=1.7 Hz, 1H), 8.73 (s, 2H, D₂O exchangeable), 8.46 (dd, J=8.7, 1.6 Hz, 1H), 8.30 (d, J=1.6 Hz, 1H), 8.20 (d, J=8.6 Hz, 1H), 8.00 (dd, J=8.8, 1.7 Hz, 1H), 7.93 (d, J=8.9 Hz, 1H), 7.33-7.25 (m, 3H), 7.21 (d, J=7.1 Hz, 1H), 6.98-6.88 (m, 1H), 5.48 (s, 2H), 5.21-4.96 (m, 1H), 3.95 (s, 3H), 3.54 (s, 2H), 1.53 (d, J=6.6 Hz, 6H); MS (ES+): 497.20 (M+1); (ES−): 495.10 (M−1).

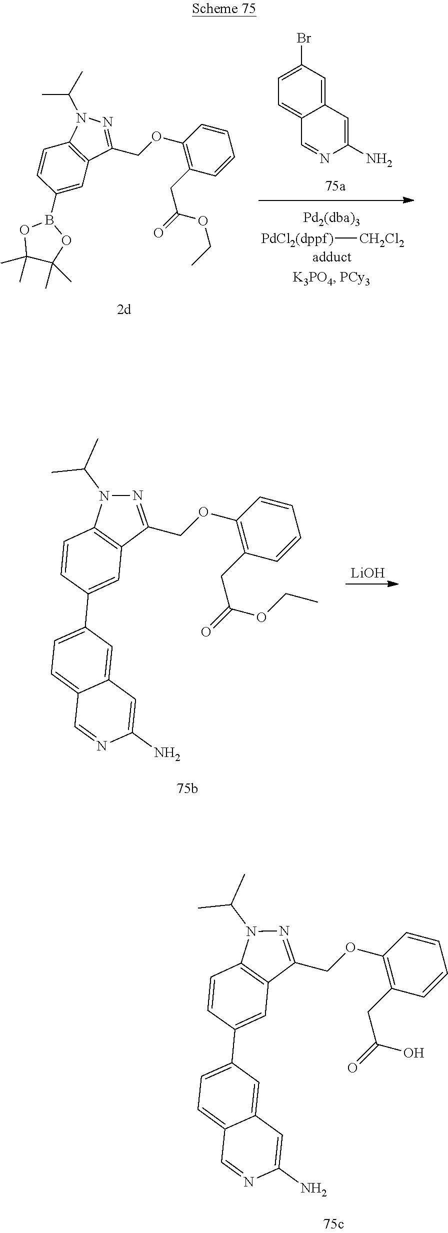

### Preparation of 2-(2-((5-(3-aminoisoquinolin-6-yl)-1-isopropyl-1H-indazol-3-yl)methoxy)phenyl)acetic acid (75c)

### Step-1: Preparation of ethyl 2-(2-((5-(3-aminoisoquinolin-6-yl)-1-isopropyl-1H-indazol-3-yl)methoxy)phenyl)acetate (75b)

Compound 75b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 6-bromoisoquinolin-3-amine (75a) (84 mg, 0.376 mmol; CAS #891785-28-7), K₃PO₄ (2M aqueous solution, 0.627 mL, 1.254 mmol), PCy₃ (17.59 mg, 0.063 mmol), PdCl₂(dppf)-CH₂Cl₂ adduct (0.0256 g, 0.031 mmol) and Pd(dba)₃ (28.7 mg, 0.031 mmol) to afford after workup and purification using method-AC, ethyl 2-(2-((5-(3-aminoisoquinolin-6-yl)-1-isopropyl-1H-indazol-3-yl)methoxy)phenyl)acetate (75b) (120 mg, 77% yield) as a clear oil; MS (ES+): 495.20 (M+1).

### Step-2: Preparation of 2-(2-((5-(3-aminoisoquinolin-6-yl)-1-isopropyl-1H-indazol-3-yl)methoxy)phenyl)acetic acid (75c)

Compound 75c was prepared according to the procedure reported in step-6 of scheme-1 from ethyl 2-(2-((5-(3-aminoisoquinolin-6-yl)-1-isopropyl-1H-indazol-3-yl)methoxy)phenyl)acetate (75b) (120 mg, 0.243 mmol) in MeOH/THF (6 mL, 1:1) using a solution of LiOH·H₂O (61.1 mg, 1.456 mmol) in water (2 mL) to afford after workup and purification twice using method-G, 2-(2-((5-(3-aminoisoquinolin-6-yl)-1-isopropyl-1H-indazol-3-yl)methoxy)phenyl)acetic acid (75c) (8 mg, 7.07% yield) as a yellow solid; ¹H NMR (300 MHz, DMSO-d₆) δ 12.07 (s, 1H, D₂O exchangeable), 8.81 (s, 1H), 8.15 (s, 1H), 7.88-7.82 (m, 4H), 7.53 (m, 1H), 7.28 (m, 2H), 7.23-7.18 (m, 1H), 6.98-6.87 (m, 1H), 6.68 (s, 1H), 5.95 (s, 2H, D₂O exchangeable), 5.48 (s, 2H), 5.13-4.95 (m, 1H), 3.54 (s, 2H), 1.52 (d, J=6.6 Hz, 6H); MS (ES+): 467.20 (M+1); (ES−): 465.10 (M−1).

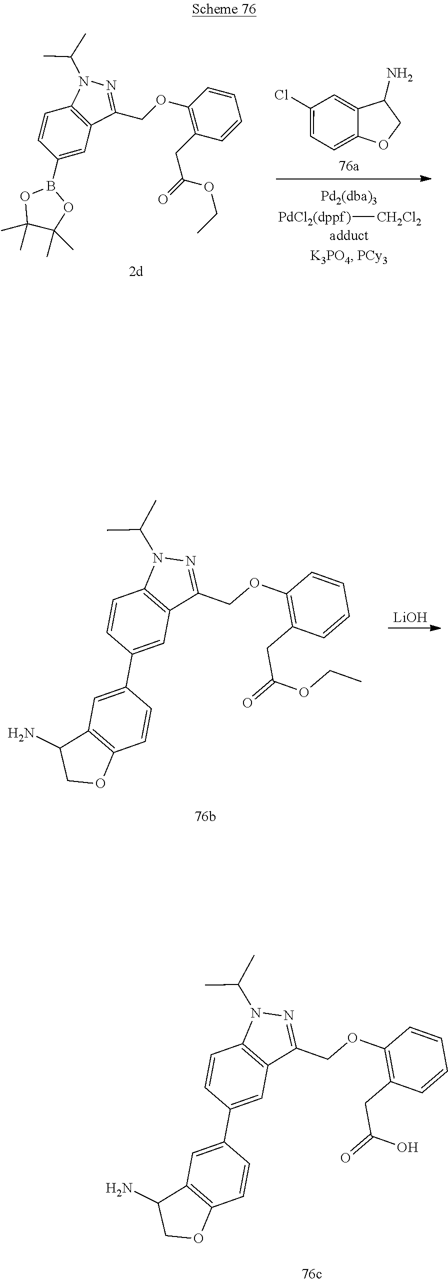

### Preparation of 2-(2-((5-(3-amino-2,3-dihydrobenzofuran-5-yl)-1-isopropyl-1H-indazol-3-yl)methoxy)phenyl)acetic acid (76c)

### Step-1: Preparation of ethyl 2-(2-((5-(3-amino-2,3-dihydrobenzofuran-5-yl)-1-isopropyl-1H-indazol-3-yl)methoxy)phenyl)acetate (76b)

Compound 76b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 5-chloro-2,3-dihydrobenzofuran-3-amine (76a) (63.8 mg, 0.376 mmol; CAS #769-21-1), K₃PO₄ (2M aqueous solution, 0.627 mL, 1.254 mmol), PCy₃ (17.59 mg, 0.063 mmol), PdCl₂(dppf)-CH₂Cl₂ adduct (0.0256 g, 0.031 mmol) and Pd₂(dba)₃ (28.7 mg, 0.031 mmol) to afford after workup and purification using method-AC, ethyl 2-(2-((5-(3-amino-2,3-dihydrobenzofuran-5-yl)-1-isopropyl-1H-indazol-3-yl)methoxy)phenyl)acetate (76b) (80 mg, 52.5% yield) as a clear oil; MS (ES+): 486.20 (M+1); 508.10 (M+Na).

### Step-2: Preparation of 2-(2-((5-(3-amino-2,3-dihydrobenzofuran-5-yl)-1-isopropyl-1H-indazol-3-yl)methoxy)phenyl)acetic acid (76c)

Compound 76c was prepared according to the procedure reported in step-6 of scheme-1 from ethyl 2-(2-((5-(3-amino-2,3-dihydrobenzofuran-5-yl)-1-isopropyl-1H-indazol-3-yl)methoxy)phenyl)acetate (76b) (80 mg, 0.165 mmol) in MeOH/THF (6 mL, 1:1) using a solution of LiOH·H₂O (41.5 mg, 0.989 mmol) in water (2 mL) to afford after workup and purification using method-G, 2-(2-((5-(3-amino-2,3-dihydrobenzofuran-5-yl)-1-isopropyl-1H-indazol-3-yl)methoxy)phenyl)acetic acid (76c) (26 mg, 34.5% yield) HCl salt as a white solid; ¹H NMR (300 MHz, DMSO-d₆) δ 8.80 (d, J=5.1 Hz, 3H, D₂O exchangeable), 8.04-7.91 (m, 2H), 7.81 (d, J=8.9 Hz, 1H), 7.71-7.61 (m, 2H), 7.32-7.23 (m, 2H), 7.20 (d, J=7.3 Hz, 1H), 7.01 (d, J=8.4 Hz, 1H), 6.96-6.87 (m, 1H), 5.43 (s, 2H), 5.15-5.07 (m, 1H), 5.07-4.96 (m, 1H), 4.83-4.68 (m, 1H), 4.65-4.49 (m, 1H), 3.52 (s, 2H), 1.51 (d, J=6.5 Hz, 6H); MS (ES+): 458.20 (M+1); (ES−): 456.10 (M−1).

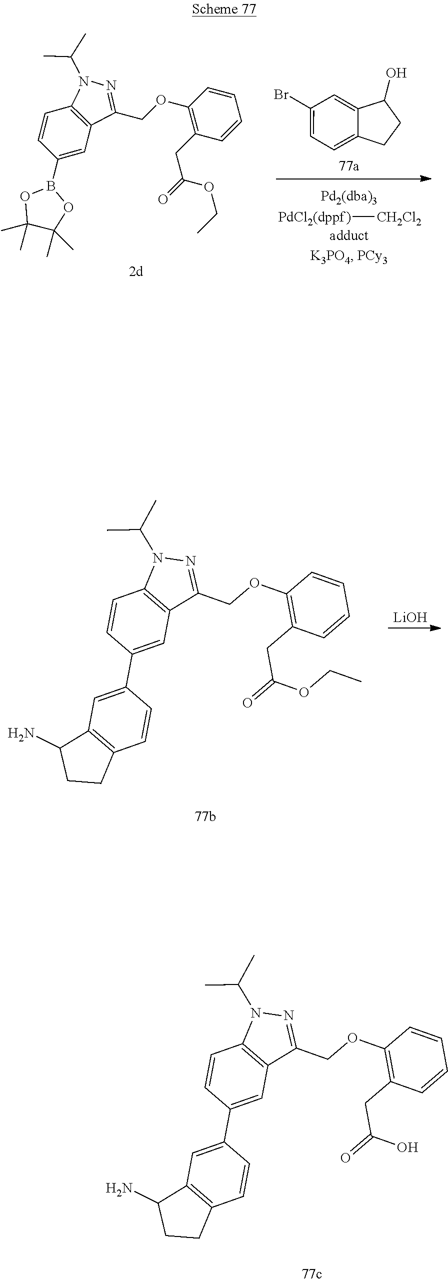

### Preparation of 2-(2-((5-(3-hydroxy-2,3-dihydro-1H-inden-5-yl)-1-isopropyl-1H-indazol-3-yl)methoxy)phenyl)acetic acid (77c)

### Step-1: Preparation of ethyl 2-(2-((5-(3-hydroxy-2,3-dihydro-1H-inden-5-yl)-1-isopropyl-1H-indazol-3-yl)methoxy)phenyl)acetate (77b)

Compound 77b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 6-bromo-2,3-dihydro-1H-inden-1-ol (77a) (80 mg, 0.376 mmol; CAS #75476-86-7), K₃PO₄ (2M aqueous solution, 0.627 mL, 1.254 mmol), PCy₃ (17.59 mg, 0.063 mmol), PdCl₂(dppf)-CH₂Cl₂ adduct (0.0256 g, 0.031 mmol) and Pd₂(dba)₃ (28.7 mg, 0.031 mmol) to afford after workup and purification using method-AC, ethyl 2-(2-((5-(3-hydroxy-2,3-dihydro-1H-inden-5-yl)˜1-isopropyl-1H-indazol-3-yl)methoxy)phenyl)acetate (77b) (80 mg, 52.7% yield) as a clear oil; MS (ES+): 485.20 (M+1); 507.20 (M+Na).

### Step-2: Preparation of 2-(2-((5-(3-hydroxy-2,3-dihydro-1H-inden-5-yl)-1-isopropyl-1H-indazol-3-yl)methoxy)phenyl)acetic acid (77c)

Compound 77c was prepared according to the procedure reported in step-6 of scheme-1 from ethyl 2-(2-((5-(3-hydroxy-2,3-dihydro-1H-inden-5-yl)-1-isopropyl-1H-indazol-3-yl)methoxy)phenyl)acetate (77b) (80 mg, 0.165 mmol) in MeOH/THF (6 mL, 1:1) using a solution of LiOH·H₂O) (41.6 mg, 0.991 mmol) in in water (2 mL) to afford after workup and purification twice using method-G, 2-(2-((5-(3-hydroxy-2,3-dihydro-1H-inden-5-yl)-1-isopropyl-1H-indazol-3-yl)methoxy)phenyl)acetic acid (77c) (38 mg, 50.4% yield) HCl salt as a white solid; H NMR (300 MHz, DMSO-d₆) δ 7.99 (s, 1H), 7.79 (d, J=8.8 Hz, 1H), 7.74-7.66 (m, 1H), 7.64 (s, 1H), 7.54 (dd, J=7.8, 1.8 Hz, 1H), 7.33-7.23 (m, 3H), 7.23-7.15 (m, 1H), 6.96-6.84 (m, 1H), 5.46 (s, 2H), 5.15-5.07 (m, 1H), 5.07-4.95 (m, 1H), 3.52 (s, 2H), 3.01-2.87 (m, 1H), 2.81-2.66 (m, 1H), 2.44-2.30 (m, 1H), 1.90-1.70 (m, 1H), 1.51 (d, J=6.5 Hz, 6H); MS (ES+): 457.20 (M+1); (ES−): 455.20 (M−1).

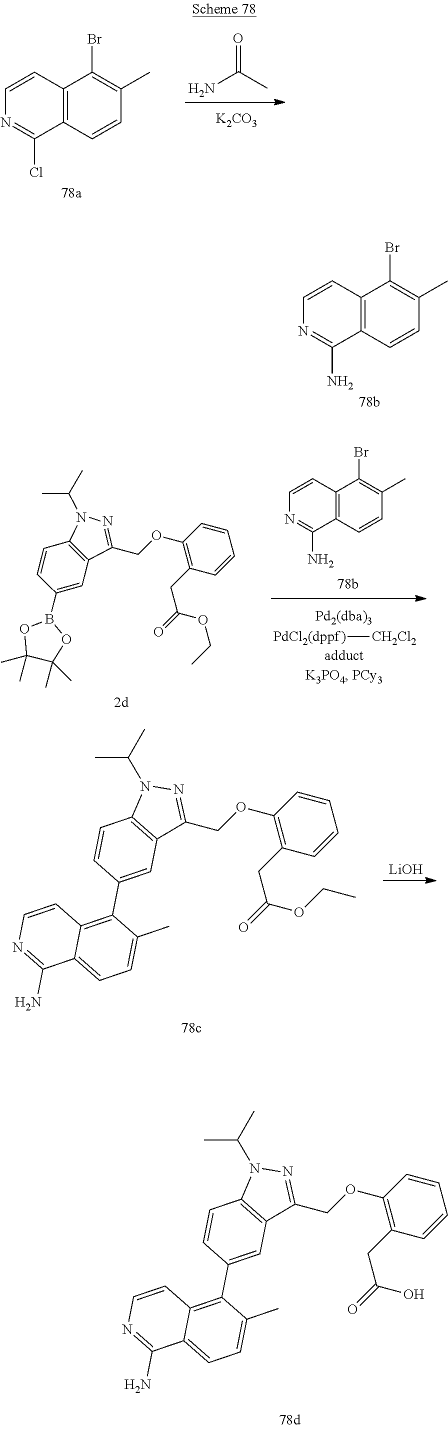

### Preparation of 2-(2-((5-(1-amino-6-methylisoquinolin-5-yl)-1-isopropyl-1H-indazol-3-yl)methoxy)phenyl)acetic acid (78d)

### Step-1: Preparation of 5-bromo-6-methylisoquinolin-1-amine (78b)

Compound 78b was prepared according to the procedure reported in step-1 of scheme-74 from 5-bromo-1-chloro-6-methylisoquinoline (78a) (300 mg, 1.169 mmol; CAS #1245647-25-9), using acetamide (1382 mg, 23.39 mmol), K₂CO₃ (485 mg, 3.51 mmol) and heating at 180° C. for 15 h. This gave after workup 5-bromo-6-methylisoquinolin-1-amine (78b) (277 mg, 100% yield) as a white solid; 1H NMR (300 MHz, DMSO-d₆) δ 8.14 (d, J=8.6 Hz, 1H), 7.89 (d, J=6.1 Hz, 1H), 7.45 (d, J=8.4 Hz, 1H), 7.08 (d, J=6.1 Hz, 1H), 6.95 (s, 2H, D₂O exchangeable), 2.53 (s, 3H); MS (ES+): 237.00 & 239.00 (M+1).

### Step-2: Preparation of ethyl 2-(2-((5-(1-amino-6-methylisoquinolin-5-yl)-1-isopropyl-1H-indazol-3-yl)methoxy)phenyl)acetate (78c)

Compound 78c was prepared according to the procedure reported in step-5 of scheme-1: to ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) was added 5-bromo-6-methylisoquinolin-1-amine (78b) (112 mg, 0.470 mmol), K₃PO₄ (2M aqueous solution, 0.627 mL, 1.254 mmol), PCy₃ (17.59 mg, 0.063 mmol), PdCl₂(dppf)-CH₂Cl₂ adduct (0.0256 g, 0.031 mmol) and Pd₂(dba)₃ (28.7 mg, 0.031 mmol) to afford after workup and purification using method-AC, ethyl 2-(2-((5-(1-amino-6-methylisoquinolin-5-yl)-1-isopropyl-1H-indazol-3-yl)methoxy)phenyl)acetate (78c) (100 mg, 0.197 mmol, 62.7% yield) as a clear oil; MS (ES+): 509.20 (M+1).

### Step-3: Preparation of 2-(2-((5-(1-amino-6-methylisoquinolin-5-yl)-1-isopropyl-1H-indazol-3-yl)methoxy)phenyl)acetic acid (78d)

Compound 78d was prepared according to the procedure reported in step-6 of scheme-1 from ethyl 2-(2-((5-(1-amino-6-methylisoquinolin-5-yl)-1-isopropyl-1H-indazol-3-yl)methoxy)phenyl)acetate (78c) (100 mg, 0.197 mmol) in MeOH/THF (6 mL, 1:1) using a solution of LiOH·H₂O (49.5 mg, 1.180 mmol) in water (2 mL) to afford after workup and purification twice using method-G, 2-(2-((5-(1-amino-6-methylisoquinolin-5-yl)-1-isopropyl-1H-indazol-3-yl)methoxy)phenyl)acetic acid (78d) (30 mg, 31.8% yield) HCl salt as a white solid; ¹H NMR (300 MHz, DMSO-d₆) δ 13.00 (s, 1H, D₂O exchangeable), 11.97 (s, 1H, D₂O exchangeable), 8.97 (s, 2H, D₂O exchangeable), 8.52 (d, J=8.6 Hz, 1H), 7.89 (d, J=8.7 Hz, 1H), 7.78 (d, J=8.6 Hz, 1H), 7.67 (s, 1H), 7.48 (d, J=7.2 Hz, 1H), 7.29-7.17 (m, 3H), 7.14 (d, J=7.3 Hz, 1H), 6.92-6.81 (m, 1H), 6.46 (d, J=7.2 Hz, 1H), 5.43 (s, 2H), 5.16-5.00 (m, 1H), 3.41 (s, 2H), 2.24 (s, 3H), 1.59-1.51 (m, 6H); MS (ES+): 481.20 (M+1); (ES−): 479.20 (M−1).

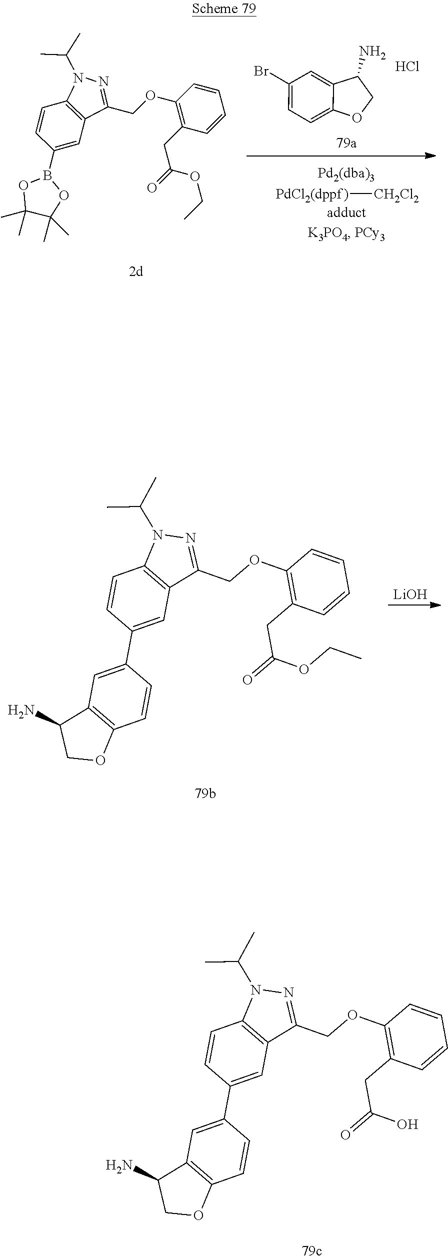

### Preparation of (S)-2-(2-((5-(3-amino-2,3-dihydrobenzofuran-5-yl)-1-isopropyl-1H-indazol-3-yl)methoxy)phenyl)acetic acid (79c)

### Step-1: Preparation of (S)-ethyl 2-(2-((5-(3-amino-2,3-dihydrobenzofuran-5-yl)-1-isopropyl-1H-indazol-3-yl)methoxy)phenyl)acetate (79b)

Compound 79b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using (S)-5-bromo-2,3-dihydrobenzofuran-3-amine hydrochloride (79a) (118 mg, 0.470 mmol; CAS #1965314-59-3), K₃PO₄ (2M aqueous solution, 0.627 mL, 1.254 mmol), PCy₃ (17.59 mg, 0.063 mmol), PdCl₂(dppf)-CH₂Cl₂ adduct (0.0256 g, 0.031 mmol) and Pd₂(dba)₃ (28.7 mg, 0.031 mmol) to afford after workup and purification using method-AC. (S)-ethyl 2-(2-((5-(3-amino-2,3-dihydrobenzofuran-5-yl)-1-isopropyl-1H-indazol-3-yl)methoxy)phenyl)acetate (79b) (100 mg, 65.7% yield) as a clear oil; MS (ES+): 486.10 (M+1); 508.20 (M+Na).

### Step-2: Preparation of (S)-2-(2-((5-(3-amino-2,3-dihydrobenzofuran-5-yl)-1-isopropyl-1H-indazol-3-yl)methoxy)phenyl)acetic acid (79c)

Compound 79c was prepared according to the procedure reported in step-6 of scheme-1 from (S)-ethyl 2-(2-((5-(3-amino-2,3-dihydrobenzofuran-5-yl)-1-isopropyl-1H-indazol-3-yl)methoxy)phenyl)acetate (79b) (100 mg, 0.206 mmol) in MeOH/THF (6 mL, 1:1) using a solution of LiOH·H₂O (51.9 mg, 1.236 mmol) in water (2 mL) to afford after workup and purification twice using method-G, (S)-2-(2-((5-(3-amino-2,3-dihydrobenzofuran-5-yl)-1-isopropyl-1H-indazol-3-yl)methoxy)phenyl)acetic acid (79c) (55 mg, 58.4% yield) HCl salt as a white solid; ¹H NMR (300 MHz, DMSO-d₆) δ 12.12 (s, 1H, D₂O exchangeable), 8.68 (s, 3H, D₂O exchangeable), 7.95 (s, 2H), 7.82 (d, J=8.9 Hz, 1H), 7.72-7.61 (m, 2H), 7.32-7.23 (m, 2H), 7.20 (d, J=7.3 Hz, 1H), 7.02 (d, J=8.4 Hz, 1H), 6.96-6.87 (m, 1H), 5.44 (s, 2H), 5.15-5.06 (m, 1H), 5.06-4.97 (m, 1H), 4.81-4.68 (m, 1H), 4.55 (dd, J=11.0, 3.5 Hz, 1H), 3.52 (s, 2H), 1.51 (d, J=6.5 Hz, 6H); MS (ES+): 458.20 (M+1); (ES−): 456.20 (M−1).

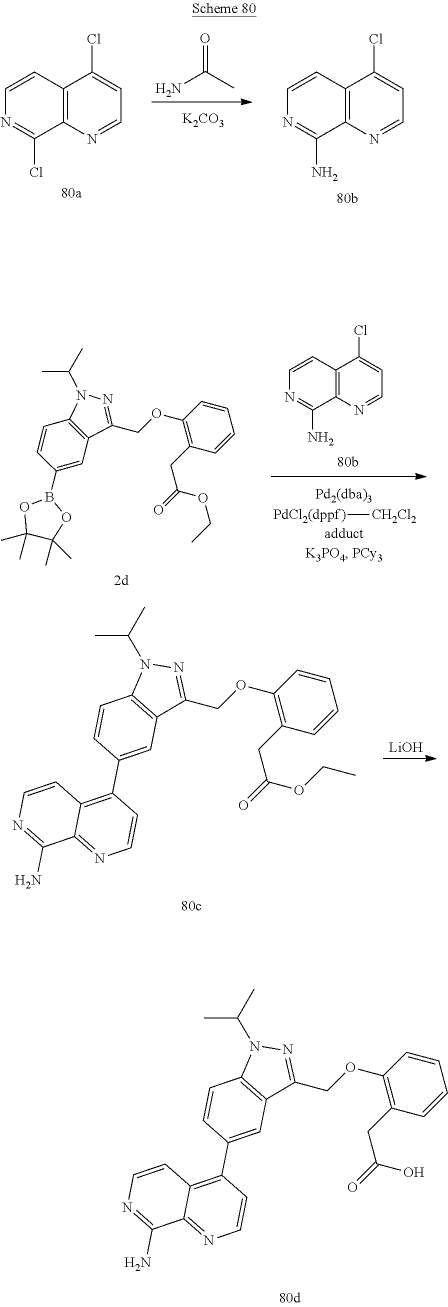

### Preparation of 2-(2-((5-(8-amino-1,7-naphthyridin-4-yl)-1-isopropyl-1H-indazol-3-yl)methoxy)phenyl)acetic acid (80d)

### Step-1: Preparation of 4-chloro-1,7-naphthyridin-8-amine (80b)

Compound 80b was prepared according to the procedure reported in step-1 of scheme-74 from 4,8-dichloro-1,7-naphthyridine (80a) (400 mg, 2.010 mmol; CAS #1279894-03-9), acetamide (2374 mg, 40.2 mmol), K₂CO₃ (833 mg, 6.03 mmol) and heating at 180° ° C. for 15 h. This gave after workup and purification using method-F, 4-chloro-1,7-naphthyridin-8-amine (80b) (80 mg, 22.16% yield) as a yellow solid; ¹H NMR (300 MHz, DMSO-d₆) δ 8.75-8.65 (m, 1H), 7.98 (d, J=5.9 Hz, 1H), 7.89 (dd, J=4.8, 1.2 Hz, 1H), 7.19 (s, 2H), 7.02 (dd, J=5.9, 1.2 Hz, 1H); MS (ES+): 180.00 & 182.10 (M+1); (ES−): 178.90 (M−1).

### Step-2: Preparation of ethyl 2-(2-((5-(8-amino-1,7-naphthyridin-4-yl)-1-isopropyl-1H-indazol-3-yl)methoxy)phenyl)acetate (80c)

Compound 80c was prepared according to the procedure reported in step-5 of scheme-1: to a degassed solution of ethyl 2-(2-((1-isopropyl-5-(4,4,5,5-tetramethyl-1,3,2-dioxaborolan-2-yl)-1H-indazol-3-yl)methoxy)phenyl)acetate (2d) (150 mg, 0.314 mmol) in dioxane/THF (4 mL, 1:1) using 4-chloro-1,7-naphthyridin-8-amine (80b) (84 mg, 0.470 mmol), K₃PO₄ (2M aqueous solution, 0.627 mL, 1.254 mmol), PCy₃ (17.59 mg, 0.063 mmol), PdCl₂(dppf)-CH₂Cl₂ adduct (0.0256 g, 0.031 mmol) and Pd₂(dba)₃ (28.7 mg, 0.031 mmol) to afford after workup and purification using method-AC, ethyl 2-(2-((5-(8-amino-1,7-naphthyridin-4-yl)-1-isopropyl-1H-indazol-3-yl)methoxy)phenyl)acetate (80c) (60 mg, 38.6% yield) as a clear oil; 1H NMR (300 MHz, DMSO-d₆) δ 8.82 (d, J=4.5 Hz, 1H), 7.91 (d, J=8.8 Hz, 2H), 7.84 (d, J=6.0 Hz, 1H), 7.66 (t, J=6.1 Hz, 1H), 7.58 (d, J=8.9 Hz, 1H), 7.31-7.22 (m, 2H), 7.17 (d, J=7.5 Hz, 1H), 7.07-6.99 (m, 2H), 6.96-6.86 (m, 2H), 5.45 (s, 2H), 5.16-5.01 (m, 1H), 4.03 (q, J=7.1 Hz, 2H), 3.52 (s, 2H), 1.54 (d, J=6.4 Hz, 6H), 0.68 (t, J=7.1 Hz, 3H); MS (ES+): 496.20 (M+1).

### Step-3: Preparation of 2-(2-((5-(8-amino-1,7-naphthyridin-4-yl)-1-isopropyl-1H-indazol-3-yl)methoxy)phenyl)acetic acid (80d)

Compound 80d was prepared according to the procedure reported in step-6 of scheme-1 from ethyl 2-(2-((5-(8-amino-1,7-naphthyridin-4-yl)-1-isopropyl-1H-indazol-3-yl)methoxy)phenyl)acetate (80c) (100 mg, 0.202 mmol) in MeOH/THF (6 mL, 1:1) using a solution of LiOH·H₂O (50.8 mg, 1.211 mmol) in water (2 mL) to afford after workup and purification twice using method-G, 2-(2-((5-(8-amino-1,7-naphthyridin-4-yl)-1-isopropyl-1H-indazol-3-yl)methoxy)phenyl)acetic acid (80d) (30 mg, 31.8% yield) HCl salt as a white solid; 1H NMR (300 MHz, DMSO-d₆) δ 13.56 (s, 1H, D₂O exchangeable), 9.58 (s, 1H, D₂O exchangeable), 9.19 (s, 1H, D₂O exchangeable), 9.05 (d, J=4.7 Hz, 1H), 8.03-7.89 (m, 3H), 7.69 (d, J=7.3 Hz, 1H), 7.56 (d, J=8.7 Hz, 1H), 7.25 (m, 2H), 7.18 (d, J=7.4 Hz, 1H), 7.05 (d, J=7.2 Hz, 1H), 6.96-6.86 (m, 1H), 5.46 (s, 2H), 5.11 (m, 1H), 3.49 (s, 2H), 1.55 (d, J=6.5 Hz, 6H); MS (ES+): 468.20 (M+1); (ES−): 466.10 (M−1).

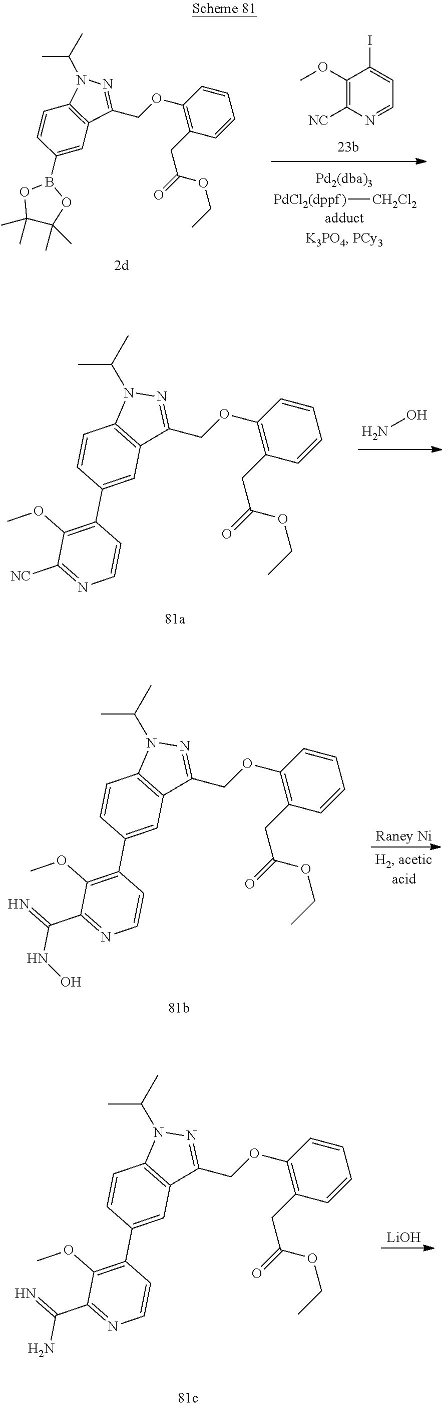

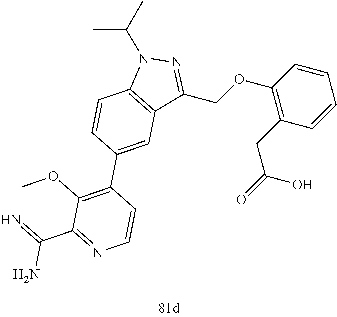 [SMILES: COc1c(-c2ccc3c(c2)c(COc2ccccc2CC(=O)O)nn3C(C)C)ccnc1C(=N)N]

### Preparation of 2-(2-((5-(2-carbamimidoyl-3-methoxypyridin-4-yl)-1-isopropyl-1H-indazol-3-yl)methoxy)phenyl)acetic acid (81d)

### Step-1: Preparation of ethyl 2-(2-((5-(2-cyano-3-methoxypyridin-4-yl)-1-isopropyl-1H-indazol-3-yl)methoxy)phenyl)acetate (81a)

Compound 81a was prepared according to the procedure reported in step-S of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (4 mL, 1:1) using 4-iodo-3-methoxypicolinonitrile (23b) (326 mg, 1.254 mmol), K₃PO₄ (2M aqueous solution, 2.090 mL, 4.18 mmol), PCy₃ (58.6 mg, 0.209 mmol), PdCl₂(dppf)-CH₂Cl₂ adduct (0.085 g, 0.105 mmol) and Pd₂(dba)₃ (96 mg, 0.105 mmol) to afford after workup and purification using method-AC, ethyl 2-(2-((5-(2-cyano-3-methoxypyridin-4-yl)-1-isopropyl-1H-indazol-3-yl)methoxy)phenyl)acetate (81a) (360 mg, 71.1% yield) as a clear oil; ¹H NMR (300 MHz, DMSO-d₆) δ 8.55 (d, J=4.9 Hz, 1H), 8.09 (s, 1H), 7.94-7.80 (m, 2H), 7.70 (dd, J=8.8, 1.6 Hz, 1H), 7.33-7.22 (m, 2H), 7.19 (d, J=7.3 Hz, 1H), 6.97-6.84 (m, 1H), 5.44 (s, 2H), 5.07 (p. J=6.6 Hz, 1H), 3.72-3.61 (m, 5H), 1.52 (d, J=6.5 Hz, 6H), 0.80 (t, J=7.1 Hz, 3H); MS (ES+): 485.20 (M+1); (ES−): 483.05 (M−1).

### Step-2: Preparation of ethyl 2-(2-((5-(2-(N-hydroxycarbamimidoyl)-3-methoxypyridin-4-yl)-1-isopropyl-1H-indazol-3-yl)methoxy)phenyl)acetate (81b)

Compound 81b was prepared according to the procedure reported in step-5 of scheme-23 from ethyl 2-(2-((5-(2-cyano-3-methoxypyridin-4-yl)-1-isopropyl-1H-indazol-3-yl)methoxy)phenyl)acetate (81a) (360 mg, 0.743 mmol) in EtOH (10 mL) using hydroxylamine (0.491 mL, 7.43 mmol) to afford after workup and purification using method-E, ethyl 2-(2-((5-(2-(N-hydroxycarbamimidoyl)-3-methoxypyridin-4-yl)˜1-isopropyl-1H-indazol-3-yl)methoxy)phenyl)acetate (81b) (300 mg, 78% yield) as a white solid; ¹H NMR (300 MHz, DMSO-d₆) δ 9.68 (s, 1H), 8.39 (d, J=4.8 Hz, 1H), 7.99 (s, 1H), 7.83 (d, J=8.8 Hz, 1H), 7.67 (d, J=8.5 Hz, 1H), 7.50 (d, J=4.8 Hz, 1H), 7.28 (d, J=4.1 Hz, 2H), 7.18 (d, J=7.3 Hz, 1H), 6.99-6.83 (m, 1H), 5.43 (s, 2H), 5.05 (p, J=6.5 Hz, 1H), 3.67 (q, J=7.1 Hz, 2H), 3.53 (s, 2H), 3.45 (s, 3H), 1.52 (d, J=6.5 Hz, 6H), 0.79 (t, J=7.1 Hz, 3H); MS (ES+): 518.20 (M+1).

### Step-3: Preparation of ethyl 2-(2-((5-(2-carbamimidoyl-3-methoxypyridin-4-yl)-1-isopropyl-1H-indazol-3-yl)methoxy)phenyl)acetate (81c)

Compound 81c was prepared according to the procedure reported in step-1 of scheme-24 from ethyl 2-(2-((5-(2-(N-hydroxycarbamimidoyl)-3-methoxypyridin-4-yl)-1-isopropyl-1H-indazol-3-yl)methoxy)phenyl)acetate (81b) (300 mg, 0.580 mmol) in EtOH (5 mL) using AcOH (0.033 mL, 0.580 mmol) and Raney Nickel (0.580 mmol) to afford after workup, ethyl 2-(2-((5-(2-carbamimidoyl-3-methoxypyridin-4-yl)-1-isopropyl-1H-indazol-3-yl)methoxy)phenyl)acetate (81c) (200 mg, 68.8% yield) as an off-white solid; MS (ES+): 502.20 (M+1).

### Step-4: Preparation of 2-(2-((5-(2-carbamimidoyl-3-methoxypyridin-4-yl)-1-isopropyl-1H-indazol-3-yl)methoxy)phenyl)acetic acid (81d)

Compound 81d was prepared according to the procedure reported in step-6 of scheme-1 from ethyl 2-(2-((5-(2-carbamimidoyl-3-methoxypyridin-4-yl)-1-isopropyl-1H-indazol-3-yl)methoxy)phenyl)acetate (81c) (200 mg, 0.399 mmol) in MeOH/THF (6 mL, 1:1) using a solution of LiOH·H₂O (100 mg, 2.392 mmol) in water (2 mL) to afford after workup and purification using method-G, 2-(2-((5-(2-carbamimidoyl-3-methoxypyridin-4-yl)-1-isopropyl-1H-indazol-3-yl)methoxy)phenyl)acetic acid (81d) (35 mg, 18.54% yield) HCl salt as a white solid; ¹H NMR (300 MHz, DMSO-d₆) δ 9.50 (s, 2H, D₂O exchangeable), 9.44 (s, 2H, D₂O exchangeable), 8.57 (d, J=4.8 Hz, 1H), 8.13 (s, 1H), 7.91 (d, J=8.8 Hz, 1H), 7.82 (d, J=4.8 Hz, 1H), 7.71 (d, J=8.8 Hz, 1H), 7.26 (d, J=4.1 Hz, 2H), 7.20 (d, J=7.4 Hz, 1H), 6.99-6.85 (m, 1H), 5.46 (s, 2H), 5.18-4.98 (m, 1H), 3.52 (s, 2H), 3.44 (s, 3H), 1.53 (d, J=6.5 Hz, 6H); MS (ES+): 474.20 (M+1); MS (ES−): 472.10 (M−1).

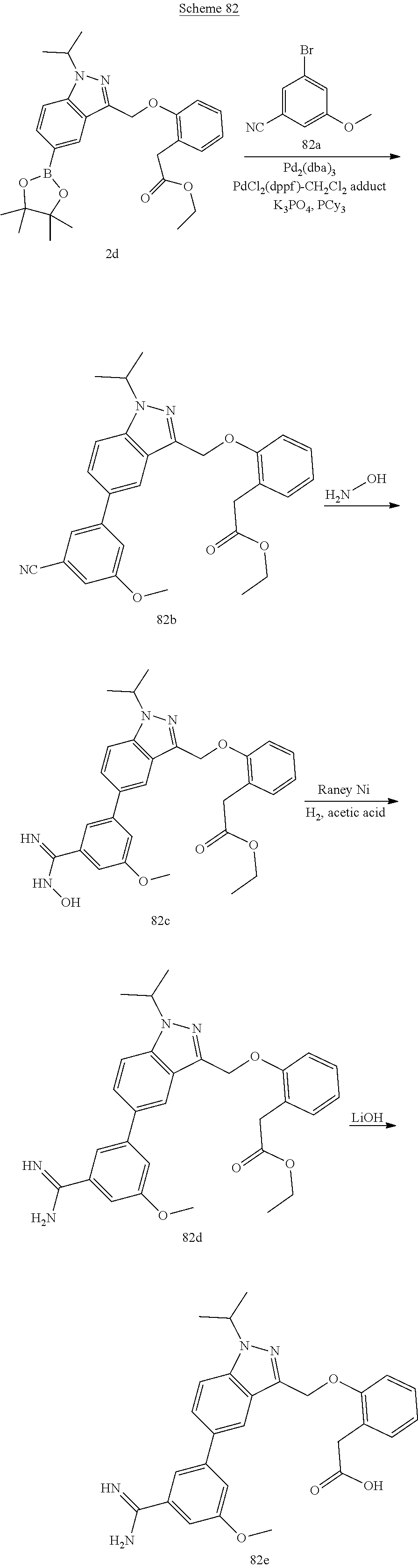

### Preparation of 2-(2-((5-(3-carbamimidoyl-5-methoxyphenyl)-1-isopropyl-1H-indazol-3-yl)methoxy)phenyl)acetic acid (82e)

### Step-1: Preparation of ethyl 2-(2-((5-(3-cyano-5-methoxyphenyl)-1-isopropyl-1H-indazol-3-yl)methoxy)phenyl)acetate (82b)

Compound 82b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (6 mL, 1:1) using 3-bromo-5-methoxybenzonitrile (82a) (332 mg, 1.568 mmol; CAS #867366-91-4), K₃PO₄ (2M aqueous solution, 2.090 mL, 4.18 mmol), PCy₃ (58.6 mg, 0.209 mmol), PdCl₂(dppf)-CH₂Cl₂ adduct (0.085 g, 0.105 mmol) and Pd₂(dba)₃ (96 mg, 0.105 mmol) to afford after workup and purification using method-AC, ethyl 2-(2-((5-(3-cyano-5-methoxyphenyl)-1-isopropyl-1H-indazol-3-yl)methoxy)phenyl)acetate (82b) (340 mg, 67.3% yield) as a clear oil; ¹H NMR (300 MHz, DMSO-d₆) δ 8.10 (s, 1H), 7.87-7.73 (m, 3H), 7.60 (s, 1H), 7.41 (s, 1H), 7.34-7.23 (m, 2H), 7.20 (d, J=7.4 Hz, 1H), 6.96-6.86 (m, 1H), 5.43 (s, 2H), 5.05 (p, J=6.7 Hz, 1H), 3.89 (s, 3H), 3.67 (q. J=7.1 Hz, 2H), 3.54 (s, 2H), 1.51 (d, J=6.5 Hz, 6H), 0.79 (t, J=7.1 Hz, 3H); MS (ES+): 484.20 (M+1).

### Step-2: Preparation of ethyl 2-(2-((5-(3-(N-hydroxycarbamimidoyl)-5-methoxyphenyl)-1-isopropyl-1H-indazol-3-yl)methoxy)phenyl)acetate (82c)

Compound 82c was prepared according to the procedure reported in step-5 of scheme-23 from ethyl 2-(2-((5-(3-cyano-5-methoxyphenyl)-1-isopropyl-1H-indazol-3-yl)methoxy)phenyl)acetate (82b) (340 mg, 0.703 mmol) in EtOH (10 mL) using hydroxylamine (0.464 mL, 7.03 mmol) to afford after workup and purification using method-E, ethyl 2-(2-((5-(3-(N-hydroxycarbamimidoyl)-5-methoxyphenyl)-1-isopropyl-1H-indazol-3-yl)methoxy)phenyl)acetate (82c) (300 mg, 83% yield) as a white solid; MS (ES+): 517.20 (M+1); (ES−): 515.10 (M−1).

### Step-3: Preparation of ethyl 2-(2-((5-(3-carbamimidoyl-5-methoxyphenyl)-1-isopropyl-1H-indazol-3-yl)methoxy)phenyl)acetate (82d)

Compound 82d was prepared according to the procedure reported in step-1 of scheme-24 from ethyl 2-(2-((5-(3-(N-hydroxycarbamimidoyl)-5-methoxyphenyl)-1-isopropyl-1H-indazol-3-yl)methoxy)phenyl)acetate (82c) (250 mg, 0.484 mmol) in EtOH (10 mL) using AcOH (0.028 mL, 0.484 mmol) and Raney Nickel (0.484 mmol) to afford after workup and purification using method-AI, ethyl 2-(2-((5-(3-carbamimidoyl-5-methoxyphenyl)-1-isopropyl-1H-indazol-3-yl)methoxy)phenyl)acetate (82d) (200 mg, 83% yield) as a colorless oil; 1H NMR (300 MHz, DMSO-d₆) δ 8.13 (s, 1H), 7.85 (s, 2H), 7.70 (s, 1H), 7.56 (s, 1H), 7.36-7.23 (m, 3H), 7.19 (d, J=7.4 Hz, 1H), 6.99-6.85 (m, 1H), 5.43 (s, 2H), 5.06 (p. J=6.6 Hz, 1H), 3.91 (s, 3H), 3.66 (q. J=7.2 Hz, 2H), 3.54 (s, 2H), 1.51 (d, J=6.5 Hz, 6H), 0.77 (t, J=7.1 Hz, 3H); MS (ES+): 501.25 (M+1).

### Step-4: Preparation of 2-(2-((5-(3-carbamimidoyl-5-methoxyphenyl)-1-isopropyl-1H-indazol-3-yl)methoxy)phenyl)acetic acid (82e)

Compound 82e was prepared according to the procedure reported in step-6 of scheme-1 from ethyl 2-(2-((5-(3-carbamimidoyl-5-methoxyphenyl)-1-isopropyl-1H-indazol-3-yl)methoxy)phenyl)acetate (82d) (200 mg, 0.400 mmol) in MeOH/THF (6 mL, 1:1) using a solution of LiOH·H₂O (101 mg, 2.397 mmol) in water (2 mL) to afford after workup and purification using method-G, 2-(2-((5-(3-carbamimidoyl-5-methoxyphenyl)-I-isopropyl-1H-indazol-3-yl)methoxy)phenyl)acetic acid (82e) (125 mg, 66.2% yield) HCl salt as a white solid; 1H NMR (300 MHz, DMSO-d₆) δ 12.06 (s, 1H, D₂O exchangeable), 9.41 (s, 2H, D₂O exchangeable), 9.09 (s, 2H, DO exchangeable), 8.19 (s, 1H), 7.86 (s, 2H), 7.72 (s, 1H), 7.63 (s, 1H), 7.34 (s, 1H), 7.31-7.24 (m, 2H), 7.20 (d, J=7.5 Hz, 1H), 6.98-6.85 (m, 1H), 5.46 (s, 2H), 5.16-4.94 (m, 1H), 3.93 (s, 3H), 3.53 (s, 2H), 1.52 (d, J=6.5 Hz, 6H); MS (ES+): 473.20 (M+1); (ES−): 471.10 (M−1).

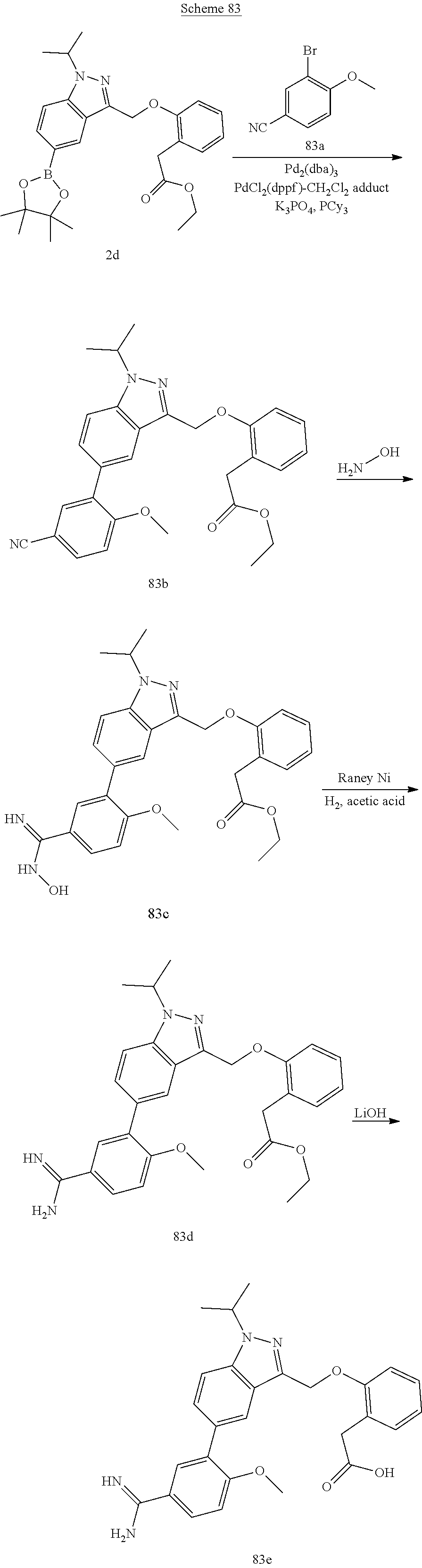

### Preparation of 2-(2-((5-(5-carbamimidoyl-2-methoxyphenyl)-1-isopropyl-1H-indazol-3-yl)methoxy)phenyl)acetic acid (83e)

### Step-1: Preparation of ethyl 2-(2-((5-(5-cyano-2-methoxyphenyl)-1-isopropyl-1H-indazol-3-yl)methoxy)phenyl)acetate (83b)

Compound 83b was prepared according to the procedure reported in step-5 of scheme-1 ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (6 mL, 1:1) using 3-bromo-4-methoxybenzonitrile (83a) (332 mg, 1.568 mmol; CAS #117572-79-9), K₃PO₄ (2M aqueous solution, 2.090 mL, 4.18 mmol), PCy₃ (58.6 mg, 0.209 mmol), PdCl₂(dppf)-CH₂Cl₂ adduct (0.085 g, 0.105 mmol) and Pd₂(dba)₃ (96 mg, 0.105 mmol) to afford after workup and purification using method-AC, ethyl 2-(2-((5-(5-cyano-2-methoxyphenyl)-1-isopropyl-1H-indazol-3-yl)methoxy)phenyl)acetate (83b) (340 mg, 67.3% yield) as a clear oil; ¹H NMR (300 MHz, DMSO-d₆) δ 7.91-7.69 (m, 4H), 7.54 (d, J=8.8 Hz, 1H), 7.38-7.22 (m, 3H), 7.19 (d, J=7.4 Hz, 1H), 7.02-6.80 (m, 1H), 5.41 (s, 2H), 5.03 (p, J=6.6 Hz, 1H), 3.85 (s, 3H), 3.67 (q, J=7.1 Hz, 2H), 3.53 (s, 2H), 1.51 (d, J=6.5 Hz, 6H), 0.83 (t, J=7.1 Hz, 3H); MS (ES+): 484.20 (M+1).

### Step-2: Preparation of ethyl 2-(2-((5-(5-(N-hydroxycarbamimidoyl)-2-methoxyphenyl)-1-isopropyl-1H-indazol-3-yl)methoxy)phenyl)acetate (83c)

Compound 83c was prepared according to the procedure reported in step-5 of scheme-23 from ethyl 2-(2-((5-(5-cyano-2-methoxyphenyl)-1-isopropyl-1H-indazol-3-yl)methoxy)phenyl)acetate (83b) in EtOH (10 mL) using hydroxylamine (0.464 mL, 7.03 mmol) to afford after workup and purification using method-E, ethyl 2-(2-((5-(5-(N-hydroxycarbamimidoyl)-2-methoxyphenyl)-1-isopropyl-1H-indazol-3-yl)methoxy)phenyl)acetate (83c) (300 mg, 83% yield) as a white solid; MS (ES+): 517.20 (M+1).

### Step-3: Preparation of ethyl 2-(2-((5-(5-carbamimidoyl-2-methoxyphenyl)-1-isopropyl-1H-indazol-3-yl)methoxy)phenyl)acetate (83d)

Compound 83d was prepared according to the procedure reported in step-1 of scheme-24 from ethyl 2-(2-((5-(5-(N-hydroxycarbamimidoyl)-2-methoxyphenyl)-1-isopropyl-1H-indazol-3-yl)methoxy)phenyl)acetate (83c) (250 mg, 0.484 mmol) in EtOH (10 mL) using AcOH (0.028 mL, 0.484 mmol) and Raney Nickel (0.484 mmol) to afford after workup and purification using method-AI, ethyl 2-(2-((5-(5-carbamimidoyl-2-methoxyphenyl)-1-isopropyl-1H-indazol-3-yl)methoxy)phenyl)acetate (83d) (200 mg, 83% yield) as a colorless oil; MS (ES+): 501.20 (M+1).

### Step-4: Preparation of 2-(2-((5-(5-carbamimidoyl-2-methoxyphenyl)-1-isopropyl-1H-indazol-3-yl)methoxy)phenyl)acetic acid (83e)

Compound 83e was prepared according to the procedure reported in step-6 of scheme-1 from ethyl 2-(2-((5-(5-carbamimidoyl-2-methoxyphenyl)-1-isopropyl-1H-indazol-3-yl)methoxy)phenyl)acetate (83d) (200 mg, 0.400 mmol) in MeOH/THF (6 mL, 1:1) using a solution of LiOH·H₂O (101 mg, 2.397 mmol) in water (2 mL) to afford after workup and purification using method-G, 2-(2-((5-(5-carbamimidoyl-2-methoxyphenyl)-1-isopropyl-1H-indazol-3-yl)methoxy)phenyl)acetic acid (83e) (70 mg, 37.1% yield) HCl salt as a white solid; ¹H NMR (300 MHz, DMSO-d₆) δ 9.20 (s, 2H, D₂O exchangeable), 8.85 (s, 2H, D₂O exchangeable), 7.95 (s, 1H), 7.91-7.82 (m, 2H), 7.78 (d, J=8.9 Hz, 1H), 7.60 (d, J=8.9 Hz, 1H), 7.34 (d, J=8.9 Hz, 1H), 7.31-7.22 (m, 2H), 7.18 (d, J=7.5 Hz, 1H), 6.97-6.84 (m, 1H), 5.42 (s, 2H), 5.11-4.95 (m, 1H), 3.88 (s, 3H), 3.51 (s, 2H), 1.52 (d, J=6.5 Hz, 6H); MS (ES+): 473.20 (M+1); (ES−): 471.15 (M−1).

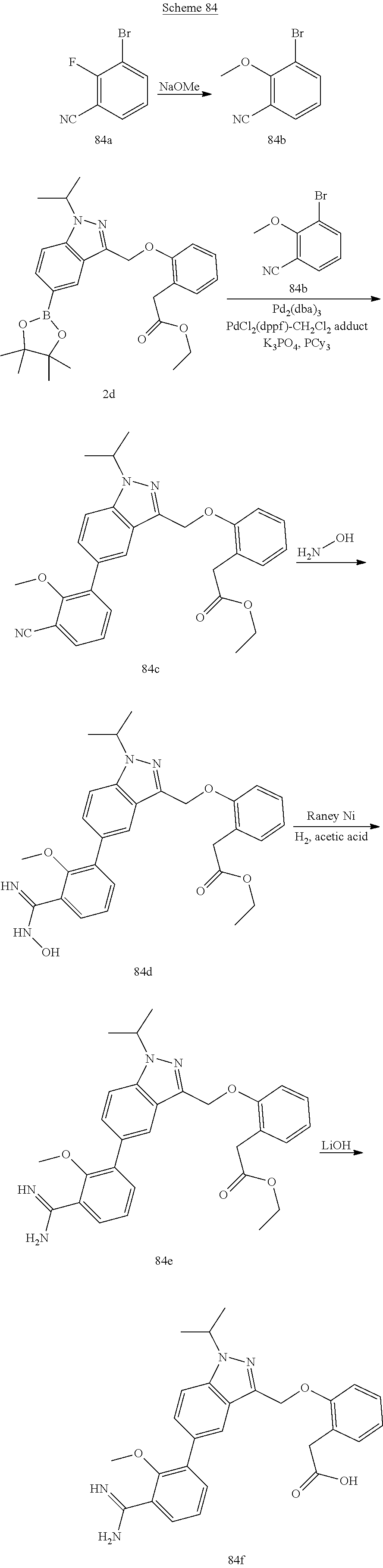

### Preparation of 2-(2-((5-(3-carbamimidoyl-2-methoxyphenyl)-1-isopropyl-1H-indazol-3-yl)methoxy)phenyl)acetic acid (841)

### Step-1: Preparation of 3-bromo-2-methoxybenzonitrile (84b)

Compound 84b was prepared according to the procedure reported in step-1 of scheme-23 from 3-bromo-2-fluorobenzonitrile (84a) (6 g, 30.0 mmol; CAS #840481-82-5) in MeOH (50 mL) using sodium methoxide (25 wt. % solution in methanol, 33.4 mL, 150 mmol) and stirring at 0° C. for 2 h and 2 h at ambient temperature. This gave after workup and purification using method-AB, 3-bromo-2-methoxybenzonitrile (84b) (3.4 g, 53.5% yield) as a white solid; 1H NMR (300 MHz, DMSO-d₆) δ 8.01 (dd, J=8.1, 1.9 Hz, 1H), 7.86 (dt, J=7.8, 1.5 Hz, 1H), 7.26 (td, J=8.0, 1.3 Hz, 1H), 3.97 (s, 3H); MS (ES+): 211.90 & 213.90 (M+1).

### Step-2: Preparation of ethyl 2-(2-((5-(3-cyano-2-methoxyphenyl)-1-isopropyl-1H-indazol-3-yl)methoxy)phenyl)acetate (84c)

Compound 84c was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (6 mL, 1:1) using 3-bromo-2-methoxybenzonitrile (84b) (332 mg, 1.568 mmol), K₃PO₄ (2M aqueous solution, 2.090 mL, 4.18 mmol), PCy₃ (58.6 mg, 0.209 mmol), PdCl₂(dppf)-CH₂Cl₂ adduct (0.085 g, 0.105 mmol) and Pd₂(dba)₃ (96 mg, 0.105 mmol) to afford after workup and purification using method-P, ethyl 2-(2-((5-(3-cyano-2-methoxyphenyl)-1-isopropyl-1H-indazol-3-yl)methoxy)phenyl)acetate (84c) (360 mg, 71.2% yield) as a clear oil; MS (ES+): 484.20 (M+1); 506.20 (M+Na); (ES−): 482.15 (M−1).

### Step-3: Preparation of ethyl 2-(2-((5-(3-(N-hydroxycarbamimidoyl)-2-methoxyphenyl)-1-isopropyl-1H-indazol-3-yl)methoxy)phenyl)acetate (84d)

Compound 84d was prepared according to the procedure reported in step-5 of scheme-23 from ethyl 2-(2-((5-(3-cyano-2-methoxyphenyl)-1-isopropyl-1H-indazol-3-yl)methoxy)phenyl)acetate (84c) (360 mg, 0.744 mmol) in EtOH (10 mL) using hydroxylamine (0.492 mL, 7.44 mmol) to afford after workup and purification using method-E, ethyl 2-(2-((5-(3-(N-hydroxycarbamimidoyl)-2-methoxyphenyl)-1-isopropyl-1H-indazol-3-yl)methoxy)phenyl)acetate (84d) (300 mg, 78% yield) as a white solid; 1H NMR (300 MHz, DMSO-d₆) δ9.49 (s, 1H), 7.88-7.83 (m, 1H), 7.80-7.74 (m, 1H), 7.63-7.57 (m, 1H), 7.46-7.35 (m, 2H), 7.29-7.25 (m, 2H), 7.19 (t, J=7.6 Hz, 2H), 6.95-6.86 (m, 1H), 5.76 (s, 2H), 5.41 (s, 2H), 5.03 (p. J=6.6 Hz, 1H), 3.67 (q, J=7.1 Hz, 2H), 3.53 (s, 2H), 3.39 (s, 3H), 1.52 (d, J=6.6 Hz, 6H), 0.81 (t, J=7.1 Hz, 3H); MS (ES+): 517.20 (M+1).

### Step-4: Preparation of ethyl 2-(2-((5-(3-carbamimidoyl-2-methoxyphenyl)-1-isopropyl-1H-indazol-3-yl)methoxy)phenyl)acetate (84e)

Compound 84e was prepared according to the procedure reported in step-1 of scheme-24 from ethyl 2-(2-((5-(3-(N-hydroxycarbamimidoyl)-2-methoxyphenyl)-1-isopropyl-1H-indazol-3-yl)methoxy)phenyl)acetate (84d) (300 mg, 0.581 mmol) in EtOH (10 mL) using AcOH (0.033 mL, 0.581 mmol) and Raney Nickel (0.581 mmol) to afford after workup and purification using method-AI, ethyl 2-(2-((5-(3-carbamimidoyl-2-methoxyphenyl)-1-isopropyl-1H-indazol-3-yl)methoxy)phenyl)acetate (84e) (250 mg, 86% yield) as a colorless oil; 1H NMR (300 MHz, DMSO-d₆) δ 7.93-7.86 (m, 1H), 7.81 (d, J=8.8 Hz, 1H), 7.65-7.55 (m, 2H), 7.48-7.43 (m, 1H), 7.32 (d, J=7.7 Hz, 1H), 7.30-7.25 (m, 2H), 7.21-7.16 (m, 1H), 6.96-6.86 (m, 1H), 5.41 (s, 2H), 5.04 (p, J=6.6 Hz, 1H), 3.70 (q, J=7.1 Hz, 2H), 3.53 (s, 2H), 3.40 (s, 3H), 1.52 (d, J=6.6 Hz, 6H), 0.80 (t, J=7.1 Hz, 3H); MS (ES+): 501.20 (M+1).

### Step-5: Preparation of 2-(2-((5-(3-carbamimidoyl-2-methoxyphenyl)-1-isopropyl-1H-indazol-3-yl)methoxy)phenyl)acetic acid (84f)

Compound 84f was prepared according to the procedure reported in step-6 of scheme-1 from ethyl 2-(2-((5-(3-carbamimidoyl-2-methoxyphenyl)-1-isopropyl-1H-indazol-3-yl)methoxy)phenyl)acetate (84e) (250 mg, 0.499 mmol) in MeOH/THF (6 mL, 1:1) using a solution of LiOH·H₂O (126 mg, 3.00 mmol) in water (2 mL) to afford after workup and purification using method-G, 2-(2-((5-(3-carbamimidoyl-2-methoxyphenyl)-1-isopropyl-1H-indazol-3-yl)methoxy)phenyl)acetic acid (84f) (70 mg, 29.7% yield) HCl salt as a white solid; 1H NMR (300 MHz, DMSO-d₆) δ 12.07 (s, 1H, D₂O exchangeable), 9.36 (s, 2H, D₂O exchangeable), 9.23-9.06 (m, 2H, D₂O exchangeable), 8.00-7.92 (m, 1H), 7.84 (d, J=8.8 Hz, 1H), 7.70 (dd, J=7.7, 1.8 Hz, 1H), 7.62 (dd, J=8.8, 1.6 Hz, 1H), 7.52 (dd, J=7.7, 1.8 Hz, 1H), 7.40 (t, J=7.7 Hz, 1H), 7.30-7.23 (m, 2H), 7.22-7.16 (m, 1H), 6.97-6.86 (m, 1H), 5.44 (s, 2H), 5.05 (p. J=6.6 Hz, 1H), 3.51 (s, 2H), 3.37 (s, 3H), 1.53 (d, J=6.6 Hz, 6H); MS (ES+): 473.20 (M+1); (ES−): 471.10 (M−1); Analysis calculated for C₂₇H₂₈N₄O₄·1.1HCl·H₂O: C, 61.11; H, 5.91; Cl, 7.35; N, 10.56; Found: C, 61.48; H, 5.95; Cl, 7.55; N, 10.30.

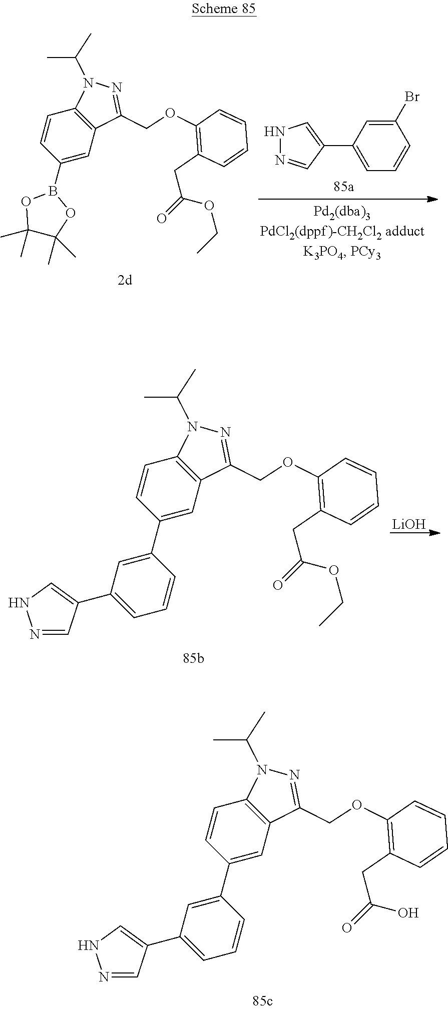

### Preparation of 2-(2-((5-(3-(1H-pyrazol-4-yl)phenyl)-1-isopropyl-1H-indazol-3-yl)methoxy)phenyl)acetic acid (85c)

### Step-1: Preparation of ethyl 2-(2-((5-(3-(1H-pyrazol-4-yl)phenyl)-1-isopropyl-1H-indazol-3-yl)methoxy)phenyl)acetate (85b)

Compound 85b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 4-(3-bromophenyl)-1H-pyrazole (85a) (187 mg, 0.836 mmol; CAS #916792-28-4), K₃PO₄ (2M aqueous solution, 0.836 mL, 1.672 mmol), PCy₃ (23.45 mg, 0.084 mmol). PdCl₂(dppf)-CH₂Cl₂ adduct (0.0341 g, 0.042 mmol) and Pd₂(dba)₃ (38.3 mg, 0.042 mmol) to afford after workup and purification using method-E, ethyl 2-(2-((5-(3-(1H-pyrazol-4-yl)phenyl)-1-isopropyl-1H-indazol-3-yl)methoxy)phenyl)acetate (85b) (25 mg, 12.09% yield) as a clear oil; 1H NMR (300 MHz, DMSO-d₆) δ 12.96 (s, 1H), 8.28 (s, 1H), 8.00 (d, J=12.6 Hz, 2H), 7.92 (d, J=2.0 Hz, 1H), 7.81-7.78 (m, 2H), 7.57 (d, J=7.6 Hz, 1H), 7.51 (d, J=7.9 Hz., 1H), 7.42 (t, J=7.6 Hz, 1H), 7.29 (d, J=4.1 Hz, 2H), 7.20 (d, J=7.3 Hz, 1H), 6.97-6.85 (m, 1H), 5.44 (s, 2H), 5.05 (p. J=6.6 Hz, 1H), 3.65 (q, J=7.1 Hz, 2H), 3.54 (s, 2H), 1.52 (d, J=6.6 Hz, 6H), 0.74 (t, J=7.1 Hz, 3H); MS (ES+): 495.20 (M+1); (ES−): 493.05 (M−1).

### Step-2: Preparation of 2-(2-((5-(3-(1H-pyrazol-4-yl)phenyl)-1-isopropyl-1H-indazol-3-yl)methoxy)phenyl)acetic acid (85c)

Compound 85c was prepared according to the procedure reported in step-6 of scheme-1 from ethyl 2-(2-((5-(3-(1H-pyrazol-4-yl)phenyl)-1-isopropyl-1H-indazol-3-yl)methoxy)phenyl)acetate (85b) (25 mg, 0.051 mmol) in THF/MeOH (4 mL) using a solution of LiOH·H₂O (12.73 mg, 0.303 mmol) in water (1 mL) to afford after workup and purification using method-G, 2-(2-((5-(3-(1H-pyrazol-4-yl)phenyl)-1-isopropyl-1H-indazol-3-yl)methoxy)phenyl)acetic acid (85c) (12 mg, 50.9% yield) HCl salt as a white solid; 1H NMR (300 MHz, DMSO-d₆) δ 8.17 (s, 2H), 8.08 (s, 1H), 7.94 (t, J=1.8 Hz, 1H), 7.84-7.74 (m, 2H), 7.58 (dt, J=7.5, 1.5 Hz, 1H), 7.55-7.47 (m, 1H), 7.43 (t, J=7.6 Hz, 1H), 7.33-7.24 (m, 2H), 7.24-7.16 (m, 1H), 6.91 (td, J=7.0, 1.9 Hz, 1H), 5.46 (s, 2H), 5.11-4.93 (m, 1H), 3.54 (s, 2H), 1.52 (d, J=6.6 Hz, 6H); MS (ES+): 467.20 (M+1); (ES−): 465.20 (M−1).

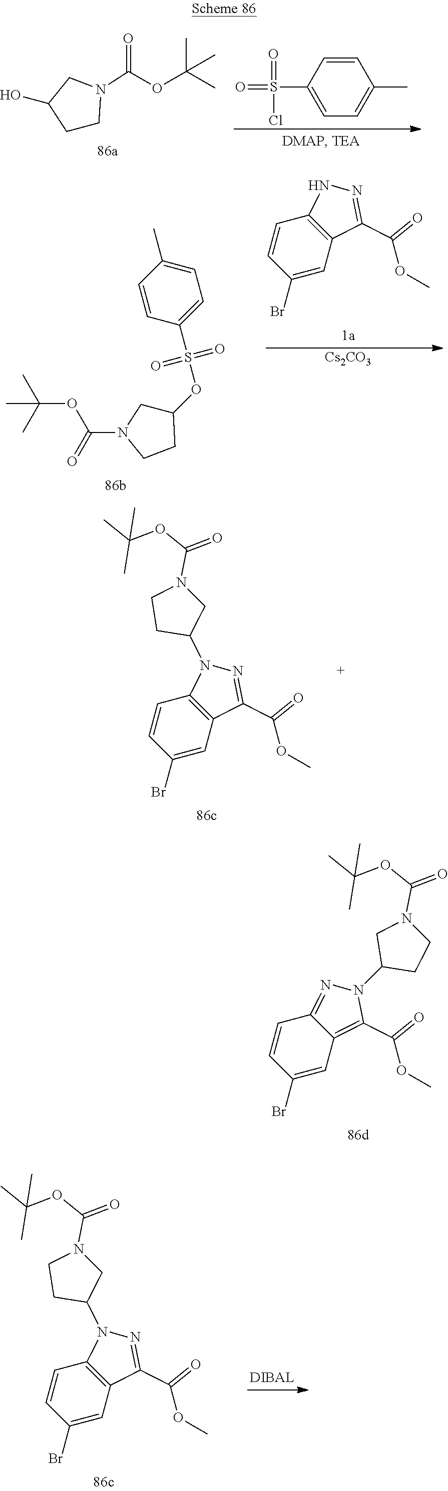

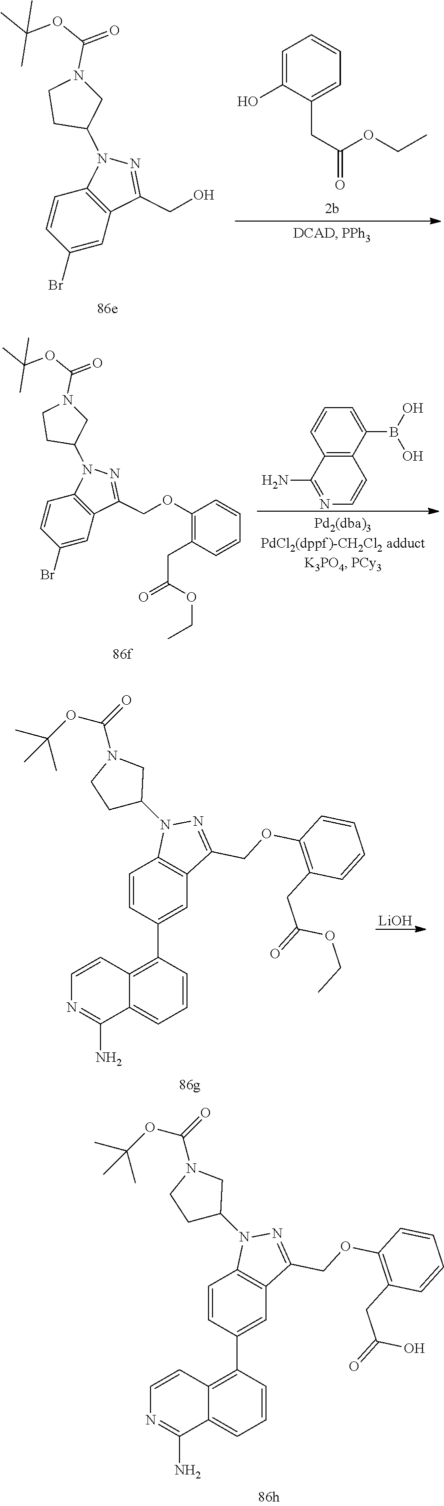

### Preparation of 2-(2-((5-(1-aminoisoquinolin-5-yl)-1-(1-(tert-butoxycarbonyl)pyrrolidin-3-yl)-1H-indazol-3-yl)methoxy)phenyl)acetic acid (86h)

### Step-1: Preparation of tert-butyl 3-(tosyloxy)pyrrolidine-1-carboxylate (86b)

To a mixture of tert-butyl 3-hydroxypyrrolidine-1-carboxylate (86a) (10 g, 53.4 mmol; CAS #: 103057-44-9), DMAP (0.326 g, 2.67 mmol) and TEA (11.17 mL, 80 mmol) in anhydrous DCM (200 mL) was added p-toluenesulfonylchloride (20.36 g, 107 mmol) at 0° C. and was stirred at 20° ° C. for 16 h. The reaction mixture was diluted with brine (20 mL) and water (20 mL). The organic layer was separated, washed with brine (20 mL), dried, filtered and concentrated in vacuum. The residue obtained was purified method-AJ to give tert-butyl 3-(tosyloxy)pyrrolidine-1-carboxylate (86b) (5.4 g, 29.6% yield) as a clear oil; H NMR (300 MHz, DMSO-d₆) δ 7.81 (dd, J=8.3, 1.6 Hz, 2H), 7.49 (d, J=7.9 Hz, 2H), 5.14-4.93 (m, 1H), 3.43-3.09 (m, 4H), 2.42 (s, 3H), 2.13-1.82 (m, 2H), 1.36 (d, J=8.4 Hz, 9H); MS (ES+): 364.10 (M+Na).

### Step-2: Preparation of methyl 5-bromo-1-(1-(tert-butoxycarbonyl)pyrrolidin-3-yl)-1H-indazole-3-carboxylate (86c) and methyl 5-bromo-2-(1-(tert-butoxycarbonyl)pyrrolidin-3-yl)-2H-indazole-3-carboxylate (86d)

To a solution of methyl 5-bromo-1H-indazole-3-carboxylate (1a) (4.03 g, 15.82 mmol) in DMF (60 mL) was added Cs₂CO₃ (10.31 g, 31.6 mmol) followed by tert-butyl 3-(tosyloxy)pyrrolidine-1-carboxylate (86b) (5.4 g, 15.82 mmol) and stirred at 60° C. for 3 h. The mixture was poured into EtOAc and washed with water and brine. The organic layer was dried, filtered and concentrated. The residue obtained was purified by method-A to give methyl 5-bromo-1-(1-(tert-butoxycarbonyl)pyrrolidin-3-yl)-1H-indazole-3-carboxylate (86c) (2.3 g, 34.3% yield) as a white solid; ¹H NMR (300 MHz, DMSO-d₆) ¿ 8.22 (dd, J=1.9, 0.7 Hz, 1H), 7.91 (dd, J=9.0, 0.7 Hz, 1H), 7.67 (dd, J=9.0, 1.9 Hz, 1H), 5.67-5.49 (m, 1H), 3.93 (s, 3H), 3.88-3.76 (m, 1H), 3.66-3.52 (m, 2H), 3.52-3.38 (m, 1H), 2.47-2.27 (m, 2H), 1.40 (d, J=8.0 Hz, 9H); MS (ES+): 446.10 & 448.10 (M+Na) and methyl 5-bromo-2-(1-(tert-butoxycarbonyl)pyrrolidin-3-yl)-2H-indazole-3-carboxylate (86d) (0.52 g, 7.75% yield) as a white solid; 1H NMR (300 MHz, DMSO-d₆) δ 8.17 (dd, J=1.9, 0.8 Hz, 1H), 7.79 (dd, J=9.1, 0.8 Hz, 1H), 7.50 (dd, J=9.1, 1.9 Hz, 1H), 6.17-5.99 (m, 1H), 4.00 (s, 3H), 3.91-3.65 (m, 2H), 3.65-3.40 (m, 2H), 2.47-2.34 (m, 2H), 1.40 (d, J=8.0 Hz, 9H); MS (ES+): 446.10 & 448.00 (M+Na).

### Step-3: Preparation of tert-butyl 3-(5-bromo-3-(hydroxymethyl)-1H-indazol-1-yl)pyrrolidine-1-carboxylate (86e)

Compound 86e was prepared according to the procedure reported in step-1 of scheme-2 from methyl 5-bromo-1-(1-(tert-butoxycarbonyl)pyrrolidin-3-yl)-1H-indazole-3-carboxylate (86c) (2.3 g, 5.42 mmol) in DCM (50 mL) using DIBAL (1M solution in DCM, 13.55 mL, 13.55 mmol) to afford after workup and purification using method-F, tert-butyl 3-(5-bromo-3-(hydroxymethyl)-1H-indazol-1-yl)pyrrolidine-1-carboxylate (86e) (1 g, 46.6% yield) as a light yellow oil; 1H NMR (300 MHz, DMSO-d₆)>8.07 (dd, J=1.8, 0.6 Hz, 1H), 7.69 (dd, J=9.0, 0.7 Hz, 1H), 7.52 (dd, J=8.9, 1.9 Hz, 1H), 5.48-5.37 (m, 1H), 5.34 (t, J=5.9 Hz, 1H, D₂O exchangeable), 4.75 (d, J=5.9 Hz, 2H), 3.81-3.70 (m, 1H), 3.62-3.37 (m, 3H), 2.41-2.19 (m, 2H), 1.40 (d, J=8.6 Hz, 9H); MS (ES+): 418.00 & 420.10 (M+Na).

### Step-4: Preparation of tert-butyl 3-(5-bromo-3-((2-(2-ethoxy-2-oxoethyl)phenoxy)methyl)-1H-indazol-1-yl)pyrrolidine-1-carboxylate (86f)

Compound 86f was prepared according to the procedure reported in step-2 of scheme-2 from tert-butyl 3-(5-bromo-3-(hydroxymethyl)-1H-indazol-1-yl)pyrrolidine-1-carboxylate (86e) (1 g, 2.52 mmol) in DCM (20 mL), using ethyl 2-(2-hydroxyphenyl)acetate (2b) (0.477 g, 2.65 mmol), PPh₃ (0.794 g, 3.03 mmol) and a solution of DCAD (1.112 g, 3.03 mmol) in DCM (20 mL) to afford after workup and purification using method-N, tert-butyl 3-(5-bromo-3-((2-(2-ethoxy-2-oxoethyl)phenoxy)methyl)-1H-indazol-1-yl)pyrrolidine-1-carboxylate (86f) (800 mg, 56.8% yield) as a white oil; ¹H NMR (300 MHz, DMSO-d₆) δ 8.00-7.94 (m, 1H), 7.75 (d, J=8.7 Hz, 1H), 7.56 (dd, J=8.9, 1.8 Hz, 1H), 7.30-7.23 (m, 2H), 7.23-7.14 (m, 1H), 6.96-6.82 (m, 1H), 5.53-5.40 (m, 1H), 5.37 (s, 2H), 3.92 (q. J=7.1 Hz, 2H), 3.84-3.73 (m, 1H), 3.63-3.55 (m, 2H), 3.54 (s, 2H), 3.50-3.36 (m, 1H), 2.44-2.22 (m, 2H), 1.41 (d, J=8.4 Hz, 9H), 0.96 (t, J=7.1 Hz, 3H); MS (ES+): 458.10 & 460.10 (M-Boc+1).

### Step-5: Preparation of tert-butyl 3-(5-(1-aminoisoquinolin-5-yl)-3-((2-(2-ethoxy-2-oxoethyl)phenoxy)methyl)-1H-indazol-1-yl)pyrrolidine-1-carboxy late (86g)

Compound 86g was prepared according to the procedure reported in step-5 of scheme-1 from tert-butyl 3-(5-bromo-3-((2-(2-ethoxy-2-oxoethyl)phenoxy)methyl)-1H-indazol-1-yl)pyrrolidine-1-carboxylate (861) (400 mg, 0.716 mmol) in dioxane/THF (6 mL, 1:1) using (1-aminoisoquinolin-5-yl)boronic acid (18a) (202 mg, 1.074 mmol), K₃PO₄ (2M aqueous solution, 1.433 mL, 2.87 mmol), PCy₃ (40.2 mg, 0.143 mmol), Pd₂(dba)₃ (65.6 mg, 0.072 mmol) and PdCl₂(dppf)-CH₂Cl₂ adduct (58.5 mg, 0.072 mmol) to afford after workup and purification using method-AM, tert-butyl 3-(5-(1-aminoisoquinolin-5-yl)-3-((2-(2-ethoxy-2-oxoethyl)phenoxy)methyl)-1H-indazol-1-yl)pyrrolidine-1-carboxylate (86g) (250 mg, 56.1% yield) as a clear oil; MS (ES+): 622.30 (M+1).

### Step-6: Preparation of 2-(2-((5-(1-aminoisoquinolin-5-yl)-1-(1-(tert-butoxycarbonyl)pyrrolidin-3-yl)-1H-indazol-3-yl)methoxy)phenyl)acetic acid (86h)

Compound 86h was prepared according to the procedure reported in step-6 of scheme-1 from tert-butyl 3-(5-(1-aminoisoquinolin-5-yl)-3-((2-(2-ethoxy-2-oxoethyl)phenoxy)methyl)-1H-indazol-1-yl)pyrrolidine-1-carboxylate (86g) (250 mg, 0.402 mmol) in MeOH/THF (6 mL, 1:1) using a solution of LiOH·H₂O (101 mg, 2.413 mmol) in water (2 mL) to afford after workup and purification using method-G, 2-(2-((5-(1-aminoisoquinolin-5-yl)-1-(1-(tert-butoxycarbonyl)pyrrolidin-3-yl)-1H-indazol-3-yl)methoxy)phenyl)acetic acid (86h) (100 mg, 41.9% yield) HCl salt as a white solid; ¹H NMR (300 MHz, DMSO-d₆) δ 13.22 (s, 1H, D₂O exchangeable), 12.02 (s, 1H, D₂O exchangeable), 9.11 (s, 2H, D₂O exchangeable), 8.61 (d, J=8.3 Hz, 1H), 7.99-7.79 (m, 4H), 7.62 (d, J=7.2 Hz, 1H), 7.55-7.46 (m, 1H), 7.29-7.21 (m, 2H), 7.21-7.13 (m, 1H), 6.98 (d, J=7.2 Hz, 1H), 6.94-6.84 (m, 1H), 5.61-5.48 (m, 1H), 5.45 (s, 2H), 3.91-3.78 (m, 1H), 3.70-3.55 (m, 2H), 3.52-3.41 (m, 3H), 2.47-2.31 (m, 2H), 1.49-1.34 (m, 9H); MS (ES+): 594.30 (M+1); (ES−): 592.20 (M−1).

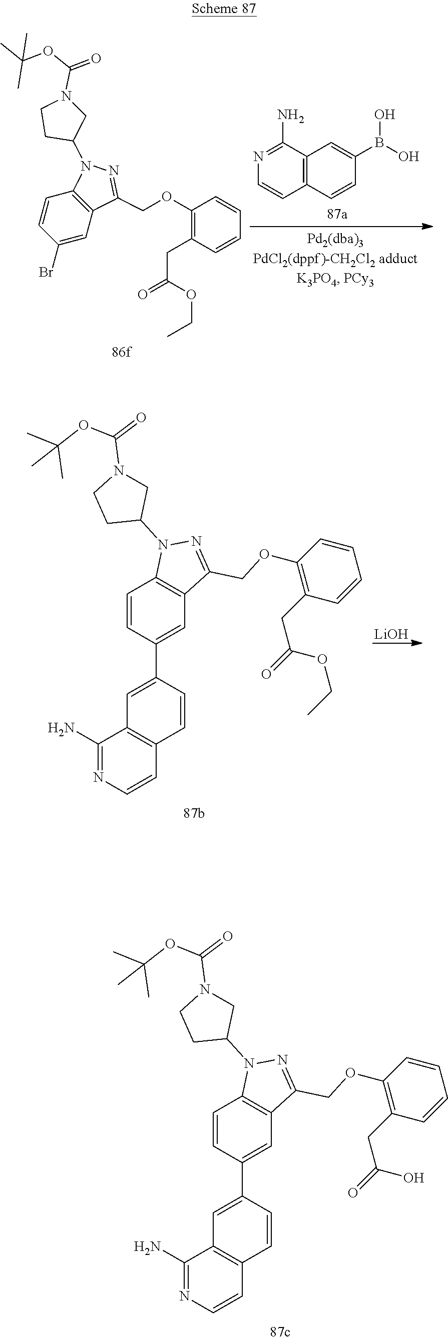

### Preparation of 2-(2-((5-(1-aminoisoquinolin-7-yl)-1-(1-(tert-butoxycarbonyl)pyrrolidin-3-yl)-1H-indazol-3-yl)methoxy)phenyl)acetic acid (87c)

### Step-1: Preparation of tert-butyl 3-(5-(1-aminoisoquinolin-7-yl)-3-((2-(2-ethoxy-2-oxoethyl)phenoxy)methyl)-1H-indazol-1-yl)pyrrolidine-1-carboxylate (87b)

Compound 87b was prepared according to the procedure reported in step-5 of scheme-1 from tert-butyl 3-(5-bromo-3-((2-(2-ethoxy-2-oxoethyl)phenoxy)methyl)-1H-indazol-1-yl)pyrrolidine-1-carboxylate (86f) (400 mg, 0.716 mmol) in dioxane/THF (6 mL, 1:1) using 1-aminoisoquinolin-7-ylboronic acid (87a) (202 mg, 1.074 mmol; CAS #2018335-61-8), K₃PO₄ (2M aqueous solution, 1.433 mL, 2.87 mmol), PCy₃ (40.2 mg, 0.143 mmol), Pd₂(dba)₃ (65.6 mg, 0.072 mmol) and PdCl₂(dppf)-CH₂Cl₂ adduct (58.5 mg, 0.072 mmol) to afford after workup and purification using method-AM, tert-butyl 3-(5-(1-aminoisoquinolin-7-yl)-3-((2-(2-ethoxy-2-oxoethyl)phenoxy)methyl)-1H-indazol-1-yl)pyrrolidine-1-carboxylate (87b) (250 mg, 56.1% yield) as a clear oil; MS (ES+): 622.30 (M+1).

### Step-2: Preparation of 2-(2-((5-(1-aminoisoquinolin-7-yl)-1-(1-(tert-butoxycarbonyl)pyrrolidin-3-yl)-1H-indazol-3-yl)methoxy)phenyl)acetic acid (87c)

Compound 87c was prepared according to the procedure reported in step-6 of scheme-1 from tert-butyl 3-(5-(1-aminoisoquinolin-7-yl)-3-((2-(2-ethoxy-2-oxoethyl)phenoxy)methyl)-1H-indazol-1-yl)pyrrolidine-1-carboxylate (87b) (250 mg, 0.402 mmol) in MeOH/THF (6 mL, 1:1) using a solution of LiOH·H₂O (101 mg, 2.413 mmol) in water (2 mL) to afford after workup and purification using method-G, 2-(2-((5-(1-aminoisoquinolin-7-yl)-1-(1-(tert-butoxycarbonyl)pyrrolidin-3-yl)-1H-indazol-3-yl)methoxy)phenyl)acetic acid (87c) (80 mg, 33.5% yield) HCl salt as a white solid; 1H NMR (300 MHz, DMSO-d₆) δ 13.23 (s, 1H, D₂O exchangeable), 12.11 (s, 1H, D₂O exchangeable), 9.12 (s, 2H, D₂O exchangeable), 8.98-8.91 (m, 1H), 8.47-8.36 (m, 1H), 8.35-8.29 (m, 1H), 8.09-7.99 (m, 2H), 7.95 (d, J=8.9 Hz, 1H), 7.69 (d, J=6.9 Hz, 1H), 7.31-7.22 (m, 3H), 7.22-7.15 (m, 1H), 6.96-6.86 (m, 1H), 5.62-5.50 (m, 1H), 5.48 (s, 2H), 3.91-3.80 (m, 1H), 3.70-3.57 (m, 2H), 3.53 (s, 2H), 3.51-3.42 (m, 1H), 2.45-2.29 (m, 2H), 1.42 (d, J=8.2 Hz, 9H); MS (ES+): 594.30 (M+1); (ES−): 592.20 (M−1).

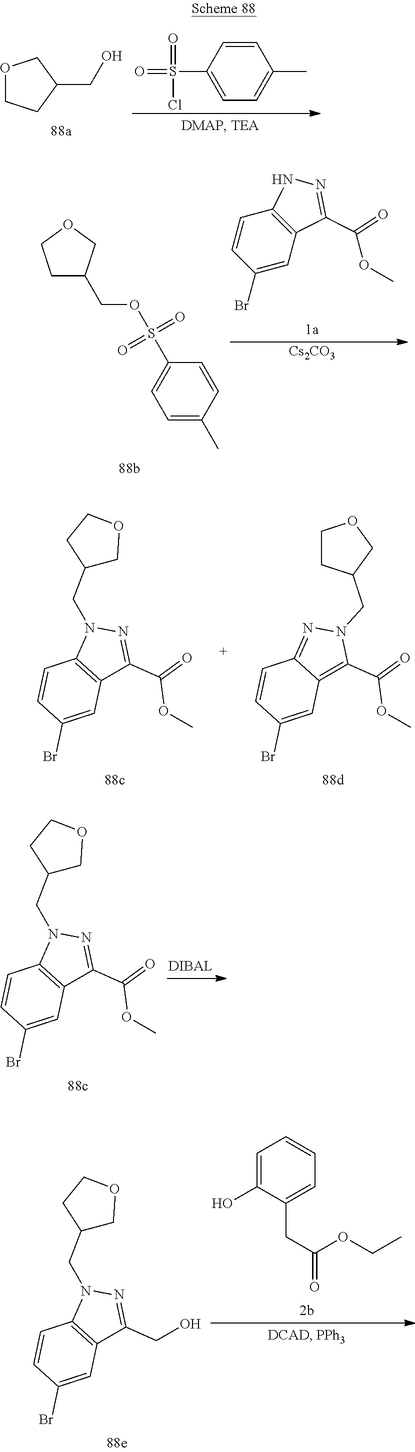

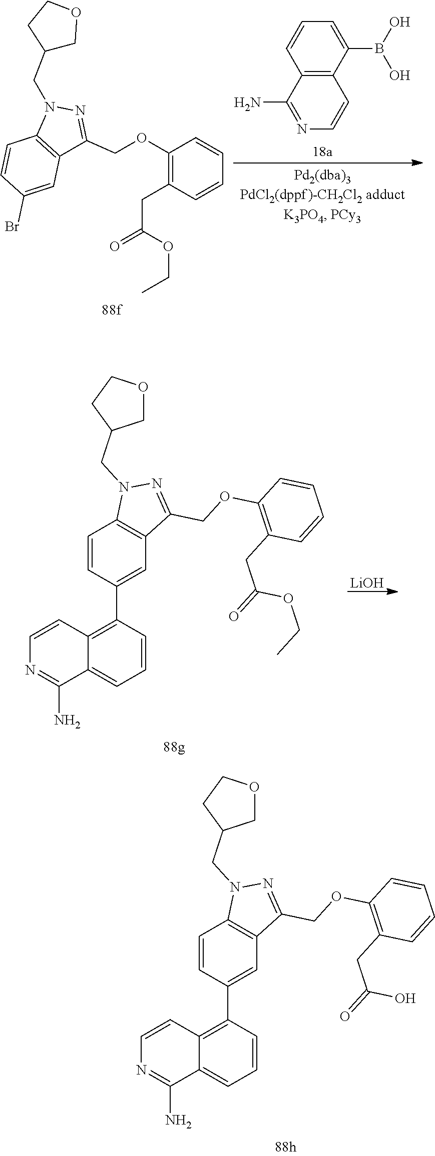 [SMILES: CCOC(=O)Cc1ccccc1OCc1nn(CC2CCOC2)c2ccc(Br)cc12 ; Nc1nccc2c(B(O)O)cccc12 ; CCOC(=O)Cc1ccccc1OCc1nn(CC2CCOC2)c2ccc(-c3cccc4c(N)nccc34)cc12 ; "LiOH" ; Nc1nccc2c(-c3ccc4c(c3)c(COc3ccccc3CC(=O)O)nn4CC3CCOC3)cccc12]

### Preparation of 2-(2-((5-(1-aminoisoquinolin-5-yl)-1-((tetrahydrofuran-3-yl)methyl)-1H-indazol-3-yl)methoxy)phenyl)acetic acid (88h)

### Step-1: Preparation of (tetrahydrofuran-3-yl)methyl 4-methylbenzenesulfonate (88b)

Compound 88b was prepared according to the procedure reported in step-1 of scheme-86 from (tetrahydrofuran-3-yl)methanol (88a) (8 g, 78 mmol; CAS #: 15833-61-1) in anhydrous DCM (200 mL) using DMAP (0.478 g, 3.92 mmol), TEA (16.38 mL, 117 mmol) and p-toluenesulfonylchloride (29.9 g, 157 mmol) to afford after workup and purification using method-AL, (tetrahydrofuran-3-yl)methyl 4-methylbenzenesulfonate (88b) (17 g, 85% yield) as a clear oil; 1H NMR (300 MHz, DMSO-d₆) δ 7.87-7.75 (m, 2H), 7.57-7.42 (m, 2H), 5.17-5.03 (m, 1H), 3.81-3.61 (m, 4H), 2.43 (s, 3H), 2.16-2.00 (m, 1H), 1.95-1.81 (m, 1H); MS (ES+): 257.10 (M+1); 279.10 (M+Na).

### Step-2: Preparation of methyl 5-bromo-1-((tetrahydrofuran-3-yl)methyl)-1H-indazole-3-carboxylate (88c) and methyl 5-bromo-2-((tetrahydrofuran-3-yl)methyl)-2H-indazole-3-carboxylate (88d)

Compound 88d was prepared according to the procedure reported in step-2 of scheme-86 from methyl 5-bromo-1H-indazole-3-carboxylate (1a) (9.95 g, 39.0 mmol) in DMF (100 mL) using Cs₂CO₃ (25.4 g, 78 mmol) and (tetrahydrofuran-3-yl)methyl 4-methylbenzenesulfonate (88b) (10 g, 39.0 mmol) to afford after workup and purification using method-A, methyl 5-bromo-1-((tetrahydrofuran-3-yl)methyl)-1H-indazole-3-carboxylate (88c) (4.9 g, 37.0% yield) as a yellow solid; 1H NMR (300 MHz, DMSO-d₆) δ 8.21 (dd, J=1.9, 0.7 Hz, 1H), 7.98-7.87 (m, 1H), 7.65 (dd, J=9.0, 1.9 Hz, 1H), 4.53 (d, J=7.5 Hz, 2H), 3.93 (s, 3H), 3.86-3.74 (m, 1H), 3.70-3.55 (m, 2H), 3.55-3.41 (m, 1H), 2.88-2.77 (m, 1H), 1.95-1.79 (m, 1H), 1.71-1.54 (m, 1H); MS (ES+): 339.00 & 341.00 (M+1); 361.00 & 363.00 (M+Na) and methyl 5-bromo-2-((tetrahydrofuran-3-yl)methyl)-2H-indazole-3-carboxylate (88d) (2.5 g, 18.89% yield) as a yellow solid; 1H NMR (300 MHz, DMSO-d₆) δ 8.18-8.11 (m, 1H), 7.83-7.75 (m, 1H), 7.53-7.45 (m, 1H), 4.96-4.70 (m, 2H), 3.99 (s, 3H), 3.88-3.72 (m, 1H), 3.69-3.59 (m, 2H), 3.59-3.50 (m, 1H), 2.98-2.76 (m, 1H), 2.01-1.80 (m, 1H), 1.77-1.51 (m, 1H); MS (ES+): 339.00 & 341.00 (M+1).

### Step-3: Preparation of (5-bromo-1-((tetrahydrofuran-3-yl)methyl)-1H-indazol-3-yl)methanol (88e)

Compound 88e was prepared according to the procedure reported in step-1 of scheme-2 from methyl 5-bromo-1-((tetrahydrofuran-3-yl)methyl)-1H-indazole-3-carboxylate (88c) (5.0 g, 14.74 mmol) in DCM (50 mL) using DIBAL (1M solution in DCM, 36.9 mL, 36.9 mmol) to afford after workup and purification using method-F, (5-bromo-1-((tetrahydrofuran-3-yl)methyl)-1H-indazol-3-yl)methanol (88e) (2.2 g, 48.0% yield) as a light yellow oil; ¹H NMR (300 MHz, DMSO-d₆) δ 8.06 (dd, J=1.9, 0.7 Hz, 1H), 7.67 (dd, J=8.9, 0.7 Hz, 1H), 7.49 (dd, J=8.9, 1.9 Hz, 1H), 5.32 (t, J=5.9 Hz, 1H, D₂O exchangeable), 4.75 (d, J=5.9 Hz, 2H), 4.37-4.28 (m, 2H), 3.85-3.72 (m, 1H), 3.69-3.55 (m, 2H), 3.55-3.40 (m, 1H), 2.87-2.66 (m, 1H), 1.94-1.78 (m, 1H), 1.70-1.53 (m, 1H); MS (ES+): 311.00 & 313.05 (M+1); 333.00 & 335.00 (M+Na).

### Step-4: Preparation of ethyl 2-(2-((5-bromo-1-((tetrahydrofuran-3-yl)methyl)-1H-indazol-3-yl)methoxy)phenyl)acetate (881)

Compound 88f was prepared according to the procedure reported in step-2 of scheme-2 from (5-bromo-1-((tetrahydrofuran-3-yl)methyl)-1H-indazol-3-yl)methanol (88e) (1 g, 3.21 mmol) in DCM (20 mL), using ethyl 2-(2-hydroxyphenyl)acetate (2b) (0.608 g, 3.37 mmol), PPh₃ (1.011 g, 3.86 mmol) and a solution of DCAD (1.416 g, 3.86 mmol) in DCM (20 mL) to afford after workup and purification using method-N, ethyl 2-(2-((5-bromo-1-((tetrahydrofuran-3-yl)methyl)-1H-indazol-3-yl)methoxy)phenyl)acetate (88f) (1.1 g, 72.3% yield) as a white oil; 1H NMR (300 MHz, DMSO-d₆) δ 8.00-7.92 (m, 1H), 7.78-7.71 (m, 1H), 7.54 (dd, J=8.9, 1.8 Hz, 1H), 7.30-7.15 (m, 3H), 6.97-6.87 (m, 1H), 5.38 (s, 2H), 4.47-4.36 (m, 2H), 3.91 (q, J=7.1 Hz, 2H), 3.84-3.73 (m, 1H), 3.68-3.56 (m, 2H), 3.53 (s, 2H), 3.52-3.43 (m, 1H), 2.86-2.68 (m, 1H), 1.95-1.79 (m, 1H), 1.72-1.47 (m, 1H), 0.95 (t, J=7.1 Hz, 3H); MS (ES+): 473.10 & 475.10 (M+1); 495.05 & 497.10 (M+Na); (ES−): 471.10 & 473.10 (M−1).

### Step-5: Preparation of ethyl 2-(2-((5-(1-aminoisoquinolin-5-yl)-1-((tetrahydrofuran-3-yl)methyl)-1H-indazol-3-yl)methoxy)phenyl)acetate (88g)

Compound 88g was prepared according to the procedure reported in step-5 of scheme-1 from ethyl 2-(2-((5-bromo-1-((tetrahydrofuran-3-yl)methyl)-1H-indazol-3-yl)methoxy)phenyl)acetate (88f) (400 mg, 0.845 mmol) in dioxane/THF (6 mL, 1:1) using (1-aminoisoquinolin-5-yl)boronic acid (18a) (238 mg, 1.268 mmol), K₃PO₄ (2M aqueous solution, 1.690 mL, 3.38 mmol), PCy₃ (47.4 mg, 0.169 mmol), Pd₂(dba)₃ (77 mg, 0.085 mmol) and PdCl₂(dppf)-CH₂Cl₂ adduct (69 mg, 0.085 mmol) to afford after workup and purification using method-AM, ethyl 2-(2-((5-(1-aminoisoquinolin-5-yl)-1-((tetrahydrofuran-3-yl)methyl)-1H-indazol-3-yl)methoxy)phenyl)acetate (88g) (220 mg, 48.5% yield) as a clear oil; MS (ES+): 537.30 (M+1).

### Step-6: Preparation of 2-(2-((5-(1-aminoisoquinolin-5-yl)-1-((tetrahydrofuran-3-yl)methyl)-1H-indazol-3-yl)methoxy)phenyl)acetic acid (88h)

Compound 88h was prepared according to the procedure reported in step-6 of scheme-1 from ethyl 2-(2-((5-(1-aminoisoquinolin-5-yl)-1-((tetrahydrofuran-3-yl)methyl)-1H-indazol-3-yl)methoxy)phenyl)acetate (88g) (220 mg, 0.410 mmol) in MeOH/THF (6 mL, 1:1) using a solution of LiOH·H₂O (103 mg, 2.46 mmol) in water (2 mL) to afford after workup and purification using method-G, 2-(2-((5-(1-aminoisoquinolin-5-yl)-1-((tetrahydrofuran-3-yl)methyl)-1H-indazol-3-yl)methoxy)phenyl)acetic acid (88h) (65 mg, 31.2% yield) HCl salt as a white solid; ¹H NMR (300 MHz, DMSO-d₆) δ 13.24 (s, 1H, D₂O exchangeable), 11.98 (s, 1H, D₂O exchangeable), 9.17 (s, 2H, D₂O exchangeable), 8.62 (d, J=8.4 Hz, 1H), 8.01-7.81 (m, 4H), 7.62 (d, J=7.3 Hz, 1H), 7.49 (dd, J=8.6, 1.7 Hz, 1H), 7.30-7.19 (m, 2H), 7.19-7.12 (m, 1H), 6.99 (d, J=7.3 Hz, 1H), 6.94-6.84 (m, 1H), 5.46 (s, 2H), 4.48 (d, J=7.4 Hz, 2H), 3.91-3.76 (m, 1H), 3.73-3.60 (m, 2H), 3.60-3.51 (m, 1H), 3.47 (s, 2H), 2.94-2.77 (m, 1H), 2.00-1.85 (m, 1H), 1.79-1.61 (m, 1H); MS (ES+): 509.20 (M+1); (ES−): 507.20 (M−1); Analysis calculated for C₃₀H₂₈N₄O₄·1.25HCl·1.5H₂O: C, 62.00; H, 5.59; Cl, 7.62; N, 9.64; Found: C, 61.78; H, 5.49; Cl, 7.75; N, 9.63.

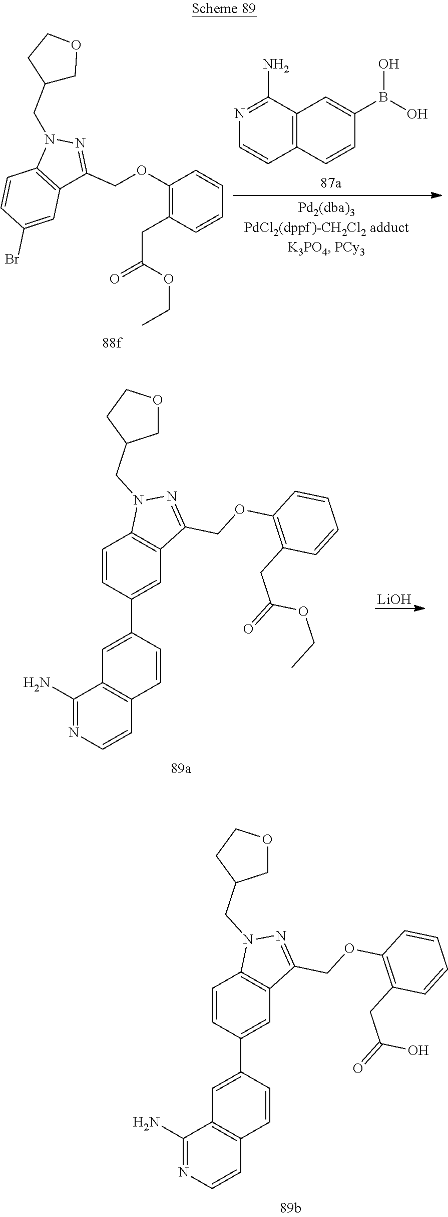

### Preparation of 2-(2-((5-(1-aminoisoquinolin-7-yl)-1-((tetrahydrofuran-3-yl)methyl)-1H-indazol-3-yl)methoxy)phenyl)acetic acid (89b)

### Step-1: Preparation of ethyl 2-(2-((5-(1-aminoisoquinolin-7-yl)-1-((tetrahydrofuran-3-yl)methyl)˜1H-indazol-3-yl)methoxy)phenyl)acetate (89a)

Compound 89a was prepared according to the procedure reported in step-S of scheme-1 from ethyl 2-(2-((5-bromo-1-((tetrahydrofuran-3-yl)methyl)-1H-indazol-3-yl)methoxy)phenyl)acetate (88f) (400 mg, 0.845 mmol) in dioxane/THF (6 ml, 1:1) using 1-aminoisoquinolin-7-ylboronic acid (87a) (238 mg, 1.268 mmol), K₃PO₄ (2M aqueous solution, 1.690 mL, 3.38 mmol), PCy₃ (47.4 mg, 0.169 mmol), Pd₂(dba)₃ (77 mg, 0.085 mmol) and PdCl₂(dppf)-CH₂Cl₂ adduct (69 mg, 0.085 mmol) to afford after workup and purification using method-AM, ethyl 2-(2-((5-(1-aminoisoquinolin-7-yl)-1-((tetrahydrofuran-3-yl)methyl)-1H-indazol-3-yl)methoxy)phenyl)acetate (89a) (220 mg, 48.5% yield) as a clear oil; MS (ES+): 537.30 (M+1).

### Step-2: Preparation of 2-(2-((5-(1-aminoisoquinolin-7-yl)-1-((tetrahydrofuran-3-yl)methyl)-1H-indazol-3-yl)methoxy)phenyl)acetic acid (89b)

Compound 89b was prepared according to the procedure reported in step-6 of scheme-1 from ethyl 2-(2-((5-(1-aminoisoquinolin-7-yl)-1-((tetrahydrofuran-3-yl)methyl)-1H-indazol-3-yl)methoxy)phenyl)acetate (89a) (220 mg, 0.410 mmol) in MeOH/THF (6 mL, 1:1) using a solution of LiOH·H₂O (103 mg, 2.46 mmol) in water (2 mL) to afford after workup and purification using method-G, 2-(2-((5-(1-aminoisoquinolin-7-yl)-1-((tetrahydrofuran-3-yl)methyl)-1H-indazol-3-yl)methoxy)phenyl)acetic acid (89b) (120 mg, 57.6% yield) HCl salt as a white solid; ¹H NMR (300 MHz, DMSO-d₆) δ 13.37 (s, 1H, D₂O exchangeable), 12.24 (s, 1H, D₂O exchangeable), 9.24 (s, 2H, D₂O exchangeable), 9.01-8.94 (m, 1H), 8.41 (dd, 1H), 8.36-8.25 (m, 1H), 8.10-7.97 (m, 2H), 7.92 (d, J=8.9 Hz, 1H), 7.69 (d, J=6.9 Hz, 1H), 7.33-7.16 (m, 4H), 6.96-6.85 (m, 1H), 5.49 (s, 2H), 4.47 (d, J=7.7 Hz, 2H), 3.87-3.76 (m, 1H), 3.71-3.59 (m, 2H), 3.57-3.48 (m, 3H), 2.92-2.74 (m, 1H), 1.98-1.83 (m, 1H), 1.75-1.56 (m, 1H); MS (ES+): 509.20 (M+1); (ES−): 507.10 (M−1); Analysis calculated for C₃₀H₂₈N₄O₄·1.2HCl·H₂O: C, 63.18; H, 5.51; Cl, 7.46; N, 9.82; Found: C, 63.49; H, 5.51; Cl, 7.13; N, 9.85.

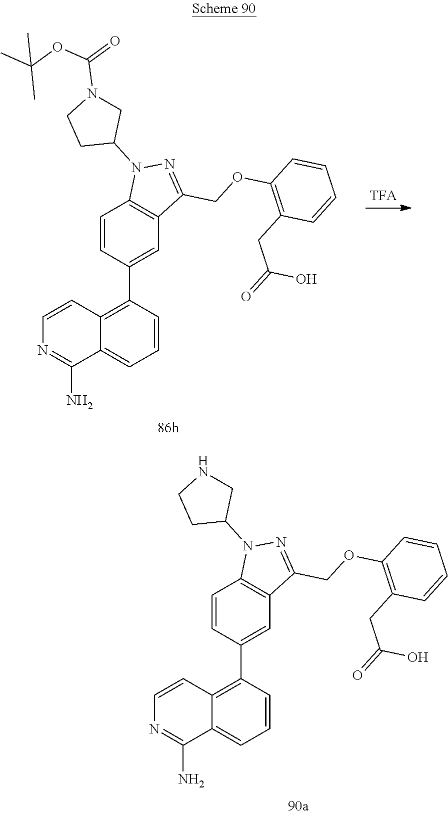 [SMILES: CC(C)(C)OC(=O)N1CCC(n2nc(COc3ccccc3CC(=O)O)c3cc(-c4cccc5c(N)nccc45)ccc32)C1 ; Nc1nccc2c(-c3ccc4c(c3)c(COc3ccccc3CC(=O)O)nn4C3CCNC3)cccc12]

### Preparation of 2-(2-((5-(1-aminoisoquinolin-5-yl)-1-(pyrrolidin-3-yl)-1H-indazol-3-yl)methoxy)phenyl)acetic acid (90a)

Compound 90a was prepared according to the procedure reported in step-4 of scheme-9 from 2-(2-((5-(1-aminoisoquinolin-5-yl)-1-(1-(tert-butoxycarbonyl)pyrrolidin-3-yl)-1H-indazol-3-yl)methoxy)phenyl)acetic acid (86h) (55 mg, 0.093 mmol) in DCM (6 mL) using TFA (0.143 mL, 1.853 mmol) to afford after workup and purification using method-G, 2-(2-((5-(1-aminoisoquinolin-5-yl)-1-(pyrrolidin-3-yl)-1H-indazol-3-yl)methoxy)phenyl)acetic acid (90a) (40 mg, 87% yield) HCl salt as a white solid; ¹H NMR (300 MHz, DMSO-d₆) δ 13.54 (s, 1H, D₂O exchangeable), 12.05 (s, 1H, DO exchangeable), 9.87 (s, 1H, DO exchangeable), 9.51 (s, 1H, D₂O exchangeable), 9.27 (s, 2H, D₂O exchangeable), 8.66 (d, J=8.3 Hz, 1H), 8.02-7.90 (m, 3H), 7.90-7.80 (m, 1H), 7.65 (d, J=7.2 Hz, 1H), 7.59-7.51 (m, 1H), 7.31-7.22 (m, 2H), 7.18 (d, J=7.3 Hz, 1H), 6.98-6.86 (m, 2H), 5.89-5.63 (m, 1H), 5.47 (s, 2H), 3.85-3.71 (m, 1H), 3.71-3.59 (m, 1H), 3.49 (m, 4H), 2.48-2.22 (m, 2H); MS (ES+): 494.20 (M+1); (ES−): 492.20 (M−1).

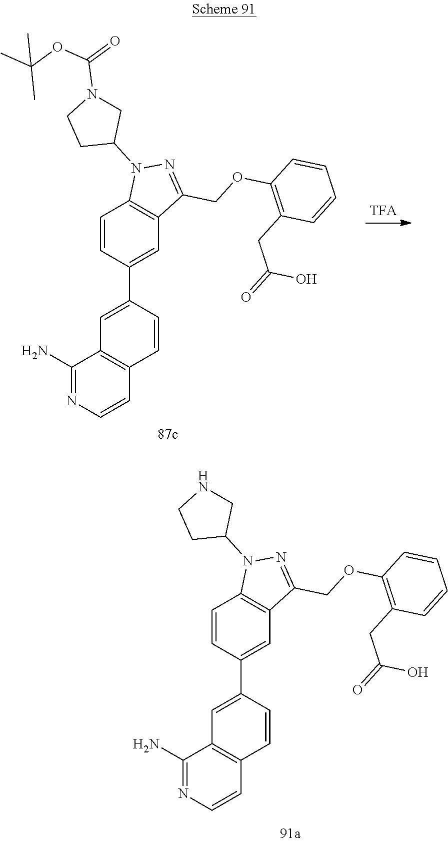 [SMILES: CC(C)(C)OC(=O)N1CCC(n2nc(COc3ccccc3CC(=O)O)c3cc(-c4ccc5ccnc(N)c5c4)ccc32)C1 ; Nc1nccc2ccc(-c3ccc4c(c3)c(COc3ccccc3CC(=O)O)nn4C3CCNC3)cc12]

### Preparation of 2-(2-((5-(1-aminoisoquinolin-7-yl)-1-(pyrrolidin-3-yl)-1H-indazol-3-yl)methoxy)phenyl)acetic acid (91a)

Compound 91a was prepared according to the procedure reported in step-4 of scheme-9 from 2-(2-((5-(1-aminoisoquinolin-7-yl)-1-(1-(tert-butoxycarbonyl)pyrrolidin-3-yl)-1H-indazol-3-yl)methoxy)phenyl)acetic acid (87c) (80 mg, 0.135 mmol) in DCM (6 mL) using TFA (0.208 mL, 2.7 mmol) to afford after workup and purification using method-G, 2-(2-((5-(1-aminoisoquinolin-7-yl)-1-(pyrrolidin-3-yl)-1H-indazol-3-yl)methoxy)phenyl)acetic acid (91a) (60 mg, 0.122 mmol, 90% yield) HCl salt as a white solid; ¹H NMR (300 MHz, DMSO-d₆) δ 13.51 (s, 1H, D₂O exchangeable), 12.13 (s, 1H, D₂O exchangeable), 9.89 (s, 1H, DO exchangeable), 9.50 (s, 2H, D₂O exchangeable), 9.33 (s, 1H, D₂O exchangeable), 9.08-8.99 (m, 1H), 8.49-8.34 (m, 2H), 8.19-8.01 (m, 2H), 8.01-7.93 (m, 1H), 7.70 (d, J=6.9 Hz, 1H), 7.39-7.13 (m, 4H), 6.98-6.86 (m, 1H), 5.79-5.66 (m, 1H), 5.51 (s, 2H), 3.88-3.70 (m, 1H), 3.70-3.59 (m, 1H), 3.59-3.35 (m, 5H), 2.48-2.18 (m, 2H); MS (ES+): 494.20 (M+1); (ES−): 492.10 (M−1).

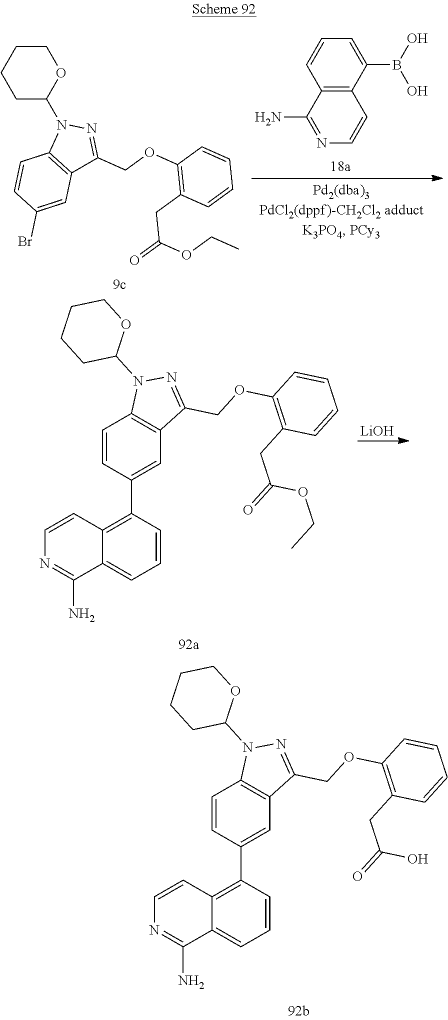

### Preparation of 2-(2-((5-(1-aminoisoquinolin-5-yl)-1-(tetrahydro-2H-pyran-2-yl)-1H-indazol-3-yl)methoxy)phenyl)acetic acid (92b)

### Step-1: Preparation of ethyl 2-(2-((5-(1-aminoisoquinolin-5-yl)-1-(tetrahydro-2H-pyran-2-yl)-1H-indazol-3-yl)methoxy)phenyl)acetate (92a)

Compound 92a was prepared according to the procedure reported in step-5 of scheme-1 from ethyl 2-(2-((5-bromo-1-(tetrahydro-2H-pyran-2-yl)-1H-indazol-3-yl)methoxy)phenyl)acetate (9c) (600 mg, 1.268 mmol) in dioxane/THF (6 mL, 1:1) using 1-aminoisoquinolin-5-ylboronic acid (18a) (357 mg, 1.901 mmol), K₃PO₄ (2M aqueous solution, 2.54 mL, 5.07 mmol), PCy₃ (71.1 mg, 0.254 mmol), Pd₂(dba)₃ (116 mg, 0.127 mmol) and PdCl₂(dppf)-CH₂Cl₂ adduct (104 mg, 0.127 mmol) to afford after workup and purification using method-AM, ethyl 2-(2-((5-(1-aminoisoquinolin-5-yl)-1-(tetrahydro-2H-pyran-2-yl)-1H-indazol-3-yl)methoxy)phenyl)acetate (92a) (650 mg, 96% yield) as a clear oil; MS (ES+): 537.30 (M+1).

### Step-2: Preparation of 2-(2-((5-(1-aminoisoquinolin-5-yl)-1-(tetrahydro-2H-pyran-2-yl)-1H-indazol-3-yl)methoxy)phenyl)acetic acid (92b)

Compound 92b was prepared according to the procedure reported in step-6 of scheme-1 from ethyl 2-(2-((5-(1-aminoisoquinolin-5-yl)-1-(tetrahydro-2H-pyran-2-yl)-1H-indazol-3-yl)methoxy)phenyl)acetate (92a) (650 mg, 1.211 mmol) in MeOH/THF (6 mL, 1:1) using a solution of LiOH·H₂O (305 mg, 7.27 mmol) in water (2 mL) to afford after workup and purification using method-G, 2-(2-((5-(1-aminoisoquinolin-5-yl)-1-(tetrahydro-2H-pyran-2-yl)-1H-indazol-3-yl)methoxy)phenyl)acetic acid (92b) (160 mg, 26.0% yield) HCl salt as a white solid; 1H NMR (300 MHz, DMSO-d₆) δ 13.16 (s, 1H, D₂O exchangeable), 12.02 (s, 1H, D₂O exchangeable), 9.15 (s, 2H, D₂O exchangeable), 8.61 (d, J=8.3 Hz, 1H), 8.01-7.81 (m, 4H), 7.61 (d, J=7.3 Hz, 1H), 7.51 (dd, J=8.6, 1.6 Hz, 1H), 7.30-7.22 (m, 2H), 7.18 (d, J=7.4 Hz, 1H), 6.99 (d, J=7.3 Hz, 1H), 6.95-6.82 (m, 1H), 6.03-5.88 (m, 1H), 5.45 (s, 2H), 3.98-3.86 (m, 1H), 3.85-3.72 (m, 1H), 3.48 (s, 2H), 2.48-2.37 (m, 1H), 2.14-1.98 (m, 2H), 1.89-1.70 (m, 1H), 1.68-1.54 (m, 2H); MS (ES+): 509.20 (M+1); (ES−): 507.20 (M−1); Analysis calculated for C₃₀H₂₈N₄O₄·HCl·1.25H₂O: C, 63.49; H, 5.59; Cl, 6.25; N, 9.87; Found: C, 63.57; H, 5.49; Cl, 6.30; N, 9.71.

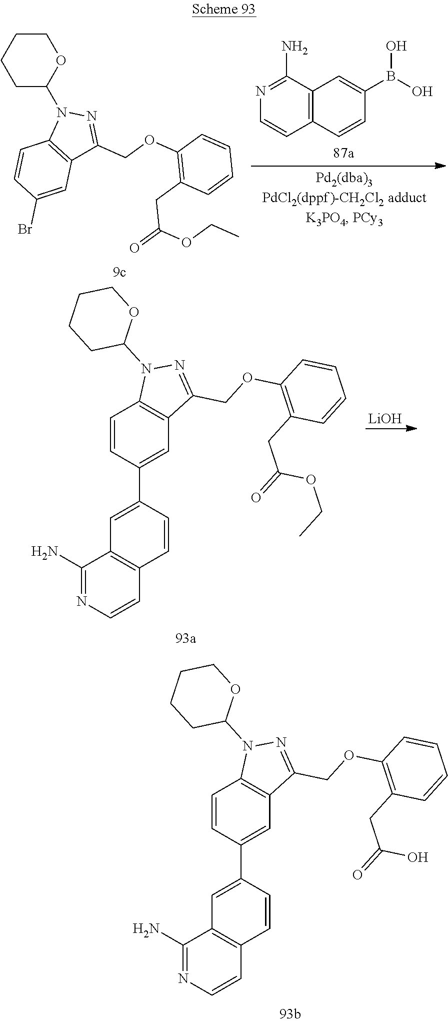

### Preparation of 2-(2-((5-(1-aminoisoquinolin-7-yl)-1-(tetrahydro-2H-pyran-2-yl)-1H-indazol-3-yl)methoxy)phenyl)acetic acid (93b)

### Step-1: Preparation of ethyl 2-(2-((5-(1-aminoisoquinolin-7-yl)-1-(tetrahydro-2H-pyran-2-yl)-1H-indazol-3-yl)methoxy)phenyl)acetate (93a)

Compound 93a was prepared according to the procedure reported in step-5 of scheme-1 from ethyl 2-(2-((5-bromo-1-(tetrahydro-2H-pyran-2-yl)-1H-indazol-3-yl)methoxy)phenyl)acetate (9c) (600 mg, 1.268 mmol) in dioxane/THF (6 mL, 1:1) was added 1-aminoisoquinolin-7-ylboronic acid (87a) (357 mg, 1.901 mmol), K₃PO₄ (2M aqueous solution, 2.54 mL, 5.07 mmol), PCy₃ (71.1 mg, 0.254 mmol), Pd₂(dba)₃ (116 mg, 0.127 mmol) and PdCl₂(dppf)-CH₂Cl₂ adduct (104 mg, 0.127 mmol) to afford after workup and purification using method-AM, ethyl 2-(2-((5-(1-aminoisoquinolin-7-yl)-1-(tetrahydro-2H-pyran-2-yl)-1H-indazol-3-yl)methoxy)phenyl)acetate (93a) (650 mg, 96% yield) as a clear oil; MS (ES+): 537.30 (M+1).

### Step-2: Preparation of 2-(2-((5-(1-aminoisoquinolin-7-yl)-1-(tetrahydro-2H-pyran-2-yl)-1H-indazol-3-yl)methoxy)phenyl)acetic acid (93b)

Compound 93b was prepared according to the procedure reported in step-6 of scheme-1 from ethyl 2-(2-((5-(1-aminoisoquinolin-7-yl)-1-(tetrahydro-2H-pyran-2-yl)-1H-indazol-3-yl)methoxy)phenyl)acetate (93a) (650 mg, 1.211 mmol) in MeOH/THF (6 mL, 1:1) using a solution of LiOH·H₂O (305 mg, 7.27 mmol) in water (2 mL) to afford after workup and purification using method-G, 2-(2-((5-(1-aminoisoquinolin-7-yl)-1-(tetrahydro-2H-pyran-2-yl)-1H-indazol-3-yl)methoxy)phenyl)acetic acid (93b) (200 mg, 32.5% yield) HCl salt as a white solid; ¹H NMR (300 MHz, DMSO-d₆) δ 13.18 (s, 1H, D₂O exchangeable), 12.10 (s, 1H, DO exchangeable), 9.12 (s, 2H, DO exchangeable), 8.95 (s, 1H), 8.42 (dd, 1H), 8.37-8.23 (m, 1H), 8.10-7.98 (m, 2H), 7.94 (d, J=8.8 Hz, 1H), 7.69 (d, J=6.9 Hz, 1H), 7.34-7.25 (m, 3H), 7.21 (d, J=7.3 Hz, 1H), 6.99-6.85 (m, 1H), 6.02-5.87 (m, 1H), 5.48 (s, 2H), 3.98-3.86 (m, 1H), 3.86-3.72 (m, 1H), 3.53 (s, 2H), 2.47-2.35 (m, 1H), 2.14-1.97 (m, 2H), 1.89-1.70 (m, 1H), 1.70-1.52 (m, 2H); MS (ES+): 509.20 (M+1); (ES−): 507.10 (M−1); Analysis calculated for C₃₀H₂₈N₄O₄·HCl·1.25H₂O: C, 63.49; H, 5.59; Cl, 6.25; N, 9.87; Found: C, 63.63; H, 5.55; Cl, 6.46; N, 9.87.

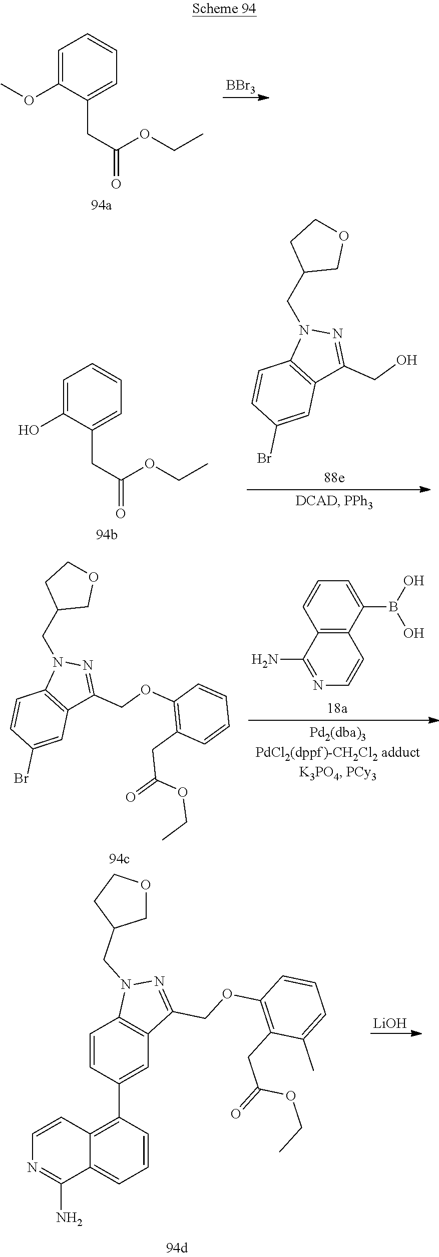

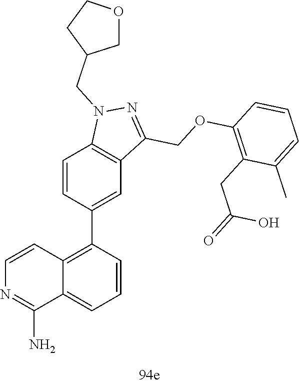 [SMILES: Cc1cccc(OCc2nn(CC3CCOC3)c3ccc(-c4cccc5c(N)nccc45)cc23)c1CC(=O)O]

### Preparation of 2-(2-((5-(1-aminoisoquinolin-5-yl)-1-((tetrahydrofuran-3-yl)methyl)-1H-indazol-3-yl)methoxy)-6-methylphenyl)acetic acid (94e)

### Step-1: Preparation of ethyl 2-(2-hydroxy-6-methylphenyl)acetate (94b)

To a stirred solution of ethyl 2-(2-methoxy-6-methylphenyl)acetate (94a) (8.0 g, 38.41 mmol; CAS #1261453-08-0) in DCM (160 mL) was added dropwise at −78° C. BBr₃ (38.49 g, 153.64 mmol) and stirred at −78 ºC for 3 h. Reaction was quenched by slow addition of ethanol (100 mL) at −20° ° C. to 10° C. and concentrated in vacuum. The residue was treated with water (200 mL), extracted with ethyl acetate (2×100 mL). The combined organic layer was washed with brine (100 mL), dried, filtered and concentrated. The obtained residue was purified by method-AN to give ethyl 2-(2-hydroxy-6-methylphenyl)acetate (94b) (2.5 g. yield 34%) as a liquid; ¹H NMR (300 MHz, DMSO-d₆) δ 9.36 (d, J=1.3 Hz, 1H), 6.94 (t, J=7.7 Hz, 1H), 6.63 (t, J=7.9 Hz, 2H), 4.05 (qd, J=7.0, 1.3 Hz, 2H), 3.59 (s, 2H), 2.16 (s, 3H), 1.17 (td, J=7.1, 1.3 Hz, 3H).

### Step-2: Preparation of ethyl 2-(2-((5-bromo-1-((tetrahydrofuran-3-yl)methyl)-1H-indazol-3-yl)methoxy)-6-methylphenyl)acetate (94c)

Compound 94c was prepared according to the procedure reported in step-2 of scheme-2 from (5-bromo-1-((tetrahydrofuran-3-yl)methyl)-1H-indazol-3-yl)methanol (88e) (1 g, 3.21 mmol) in DCM (20 mL), using ethyl 2-(2-hydroxy-6-methylphenyl)acetate (94b) (0.655 g, 3.37 mmol). PPh₃ (1.011 g, 3.86 mmol) and a solution of DCAD (1.416 g, 3.86 mmol) in DCM (20 mL) to afford after workup and purification using method-N, ethyl 2-(2-((5-bromo-1-((tetrahydrofuran-3-yl)methyl)-1H-indazol-3-yl)methoxy)-6-methylphenyl)acetate (94c) (1.1 g, 70.2% yield) as a colorless oil; 1H NMR (300 MHz, DMSO-d₆) δ 7.95 (d, J=1.7 Hz, 1H), 7.75 (d, J=8.9 Hz, 1H), 7.54 (dd, J=8.9, 1.9 Hz, 1H), 7.19-7.03 (m, 2H), 6.81 (d, J=7.2 Hz, 1H), 5.37 (s, 2H), 4.46-4.37 (m, 2H), 3.95 (q. J=7.1 Hz, 2H), 3.85-3.73 (m, 1H), 3.68-3.55 (m, 4H), 3.51-3.42 (m, 1H), 2.86-2.67 (m, 1H), 2.19 (s, 3H), 1.93-1.77 (m, 1H), 1.70-1.52 (m, 1H), 1.00 (t, J=7.1 Hz, 3H); MS (ES+): 487.10 & 489.10 (M+1).

### Step-3: Preparation of ethyl 2-(2-((5-(1-aminoisoquinolin-5-yl)-1-((tetrahydrofuran-3-yl)methyl)-1H-indazol-3-yl)methoxy)-6-methylphenyl)acetate (94d)

Compound 94d was prepared according to the procedure reported in step-5 of scheme-1 from ethyl 2-(2-((5-bromo-1-((tetrahydrofuran-3-yl)methyl)-1H-indazol-3-yl)methoxy)-6-methylphenyl)acetate (94c) (550 mg, 1.128 mmol) in dioxane/THF (6 mL, 1:1) using 1-aminoisoquinolin-5-ylboronic acid (18a) (318 mg, 1.693 mmol), K₃PO₄ (2M aqueous solution, 2.257 mL, 4.51 mmol), PCy₃ (63.3 mg, 0.226 mmol), Pd₂(dba)₃ (103 mg, 0.113 mmol), and PdCl₂(dppf)-CH₂Cl₂ adduct (92 mg, 0.113 mmol) to afford after workup and purification using method-AM, ethyl 2-(2-((5-(1-aminoisoquinolin-5-yl)-1-((tetrahydrofuran-3-yl)methyl)-1H-indazol-3-yl)methoxy)-6-methylphenyl)acetate (94d) (330 mg, 53.1% yield) as a clear oil; MS (ES+): 551.30 (M+1).

### Step-4: Preparation of 2-(2-((5-(1-aminoisoquinolin-5-yl)-1-((tetrahydrofuran-3-yl)methyl)-1H-indazol-3-yl)methoxy)-6-methylphenyl)acetic acid (94e)

Compound 94e was prepared according to the procedure reported in step-6 of scheme-1 from ethyl 2-(2-((5-(1-aminoisoquinolin-5-yl)-1-((tetrahydrofuran-3-yl)methyl)-1H-indazol-3-yl)methoxy)-6-methylphenyl)acetate (94d) (330 mg, 0.599 mmol) in MeOH/THF (6 mL, 1:1) using a solution of LiOH·H₂O (151 mg, 3.60 mmol) in water (2 mL) to afford after workup and purification using method-G, 2-(2-((5-(1-aminoisoquinolin-5-yl)-1-((tetrahydrofuran-3-yl)methyl)-1H-indazol-3-yl)methoxy)-6-methylphenyl)acetic acid (94e) (150 mg, 47.9% yield) as a white solid; 1H NMR (300 MHz, DMSO-d₆) δ 13.34 (s, 1H, D₂O exchangeable), 12.04 (s, 1H, D₂O exchangeable), 9.20 (s, 2H, D₂O exchangeable), 8.63 (d, J=8.3 Hz, 1H), 7.98-7.80 (m, 4H), 7.63 (d, J=7.3 Hz, 1H), 7.48 (dd, J=8.7, 1.6 Hz, 1H), 7.16-7.04 (m, 2H), 6.97 (d, J=7.3 Hz, 1H), 6.82-6.70 (m, 1H), 5.42 (s, 2H), 4.47 (d, J=7.4 Hz, 2H), 3.90-3.75 (m, 1H), 3.71-3.59 (m, 2H), 3.59-3.48 (m, 3H), 2.93-2.76 (m, 1H), 2.16 (s, 3H), 1.98-1.85 (m, 1H), 1.76-1.60 (m, 1H); MS (ES+): 523.30 (M+1); (ES−): 521.20 (M−1); Analysis calculated for C₃₁H₃₀N₄O₄·HCl·H₂O: C, 64.52; H, 5.76; Cl, 6.14; N, 9.71; Found: C, 64.51; H, 5.69; Cl, 6.31; N, 9.64.

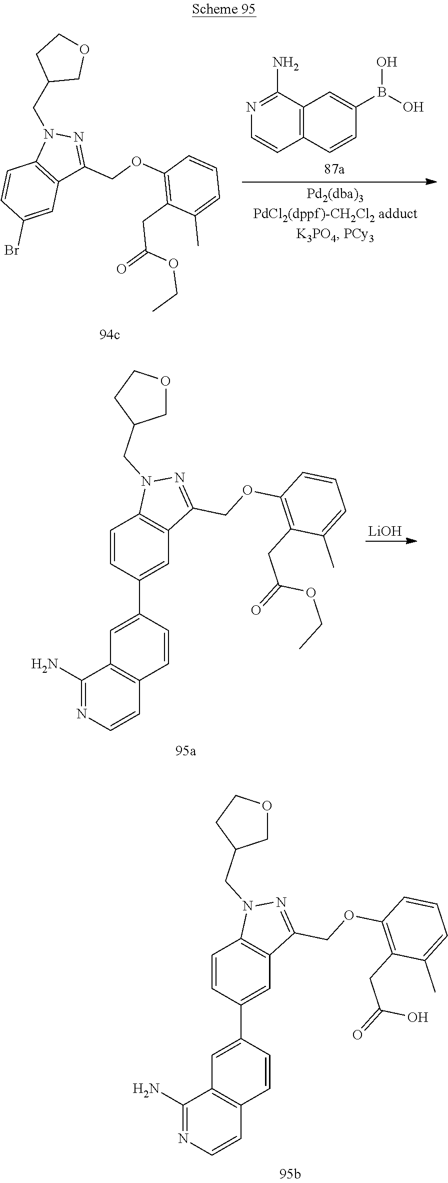

### Preparation of 2-(2-((5-(1-aminoisoquinolin-7-yl)-1-((tetrahydrofuran-3-yl)methyl)-1H-indazol-3-yl)methoxy)-6-methylphenyl)acetic acid (95b)

### Step-1: Preparation of ethyl 2-(2-((5-(1-aminoisoquinolin-7-yl)-1-((tetrahydrofuran-3-yl)methyl)-1H-indazol-3-yl)methoxy)-6-methylphenyl)acetate (95a)

Compound 95a was prepared according to the procedure reported in step-5 of scheme-1 from ethyl 2-(2-((5-bromo-1-((tetrahydrofuran-3-yl)methyl)-1H-indazol-3-yl)methoxy)-6-methylphenyl)acetate (94c) (550 mg, 1.128 mmol) in dioxane/THF (6 mL, 1:1) using 1-aminoisoquinolin-7-ylboronic acid (87a) (318 mg, 1.693 mmol), K₃PO₄ (2M aqueous solution, 2.257 mL, 4.51 mmol), PCy₃ (63.3 mg, 0.226 mmol), Pd₂(dba)₃ (103 mg, 0.113 mmol), and PdCl₂(dppf)-CH₂Cl₂ adduct (92 mg, 0.113 mmol) to afford after workup and purification using method-AM, ethyl 2-(2-((5-(1-aminoisoquinolin-7-yl)-1-((tetrahydrofuran-3-yl)methyl)-1H-indazol-3-yl)methoxy)-6-methylphenyl)acetate (95a) (330 mg, 53.1% yield) as a clear oil; MS (ES+): 551.30 (M+1).

### Step-2: Preparation of 2-(2-((5-(1-aminoisoquinolin-7-yl)-1-((tetrahydrofuran-3-yl)methyl)-1H-indazol-3-yl)methoxy)-6-methylphenyl)acetic acid (95b)

Compound 95b was prepared according to the procedure reported in step-6 of scheme-1 from ethyl 2-(2-((5-(1-aminoisoquinolin-7-yl)-1-((tetrahydrofuran-3-yl)methyl)-1H-indazol-3-yl)methoxy)-6-methylphenyl)acetate (95a) (330 mg, 0.599 mmol) in MeOH/THF (6 mL, 1:1) using a solution of LiOH·H₂O (151 mg, 3.60 mmol) in water (2 mL) to afford after workup and purification using method-G, 2-(2-((5-(1-aminoisoquinolin-7-yl)-1-((tetrahydrofuran-3-yl)methyl)-1H-indazol-3-yl)methoxy)-6-methylphenyl)acetic acid (95b) (150 mg, 47.9% yield) HCl salt as a white solid; 1H NMR (300 MHz, DMSO-d₆) δ 13.34 (s, 1H, D₂O exchangeable), 12.13 (s, 1H, D₂O exchangeable), 9.19 (s, 2H, D₂O exchangeable), 8.99-8.92 (m, 1H), 8.39 (dd, 1H), 8.34-8.26 (m, 1H), 8.11-7.98 (m, 2H), 7.98-7.89 (m, 1H), 7.69 (d, J=6.9 Hz, 1H), 7.27 (d, J=6.9 Hz, 1H), 7.17-7.09 (m, 2H), 6.85-6.74 (m, 1H), 5.47 (s, 2H), 4.48 (d, J=7.4 Hz, 2H), 3.87-3.74 (m, 1H), 3.70-3.60 (m, 2H), 3.58 (s, 2H), 3.56-3.49 (m, 1H), 2.91-2.69 (m, 1H), 2.18 (s, 3H), 2.00-1.81 (m, 1H), 1.74-1.57 (m, 1H); MS (ES+): 523.20 (M+1); (ES−): 521.10 (M−1); Analysis calculated for C₃₁H₃₀N₄O₄·HCl·1.25H₂O: C, 64.02; H, 5.81; Cl, 6.10; N, 9.63; Found: C, 63.97; H, 5.73; Cl, 5.87; N, 9.65.

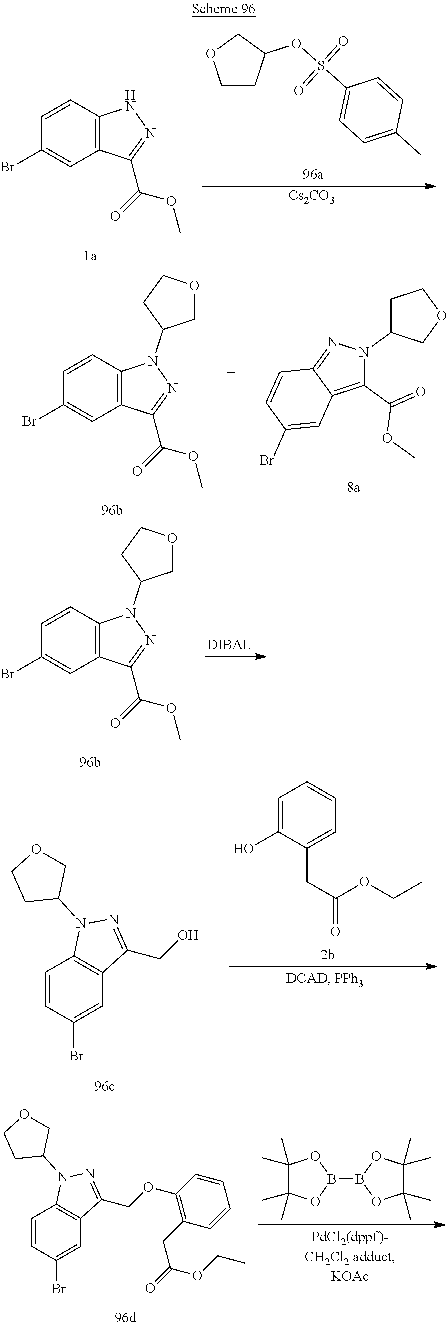

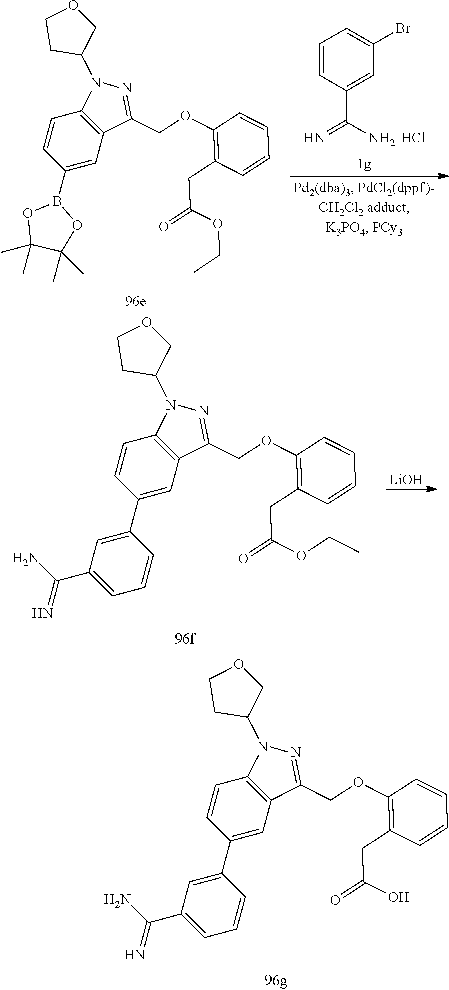

### Preparation of 2-(2-((5-(3-carbamimidoylphenyl)-1-(tetrahydrofuran-3-yl)-1H-indazol-3-yl)methoxy)phenyl)acetic acid (96g)

### Step-1: Preparation of methyl 5-bromo-1-(tetrahydrofuran-3-yl)-1H-indazole-3-carboxylate (96b) and methyl 5-bromo-2-(tetrahydrofuran-3-yl)-2H-indazole-3-carboxylate (8a)

Compounds 96b and 8a were prepared according to the procedure reported in step-2 of scheme-86 from methyl 5-bromo-1H-indazole-3-carboxylate (1a) (6.51 g, 25.5 mmol) in DMF (60 mL) using Cs₂CO₃ (16.63 g, 51.0 mmol) and tetrahydrofuran-3-yl 4-methylbenzenesulfonate (96a) (6.8 g, 28.1 mmol, CAS #13694-84-3) to afford after workup and purification using method-AO, methyl 5-bromo-1-(tetrahydrofuran-3-yl)-1H-indazole-3-carboxylate (96b) (3.7 g, 44.6% yield) as a white solid; 1H NMR (300 MHz, DMSO-d₆) & 8.21 (d, J=1.9 Hz, 1H), 7.90 (d, J=9.0 Hz, 1H), 7.66 (dd, J=9.0, 1.9 Hz, 1H), 5.70-5.57 (m, 1H), 4.18-3.95 (m, 3H), 3.93 (s, 3H), 3.91-3.81 (m, 1H), 2.49-2.43 (m, 1H), 2.40-2.26 (m, 1H); MS (ES+): 325.00 & 327.00 (M+1); 347.00 & 349.00 (M+Na) and methyl 5-bromo-2-(tetrahydrofuran-3-yl)-2H-indazole-3-carboxylate (8a) (0.82 g, 9.88% yield) as a white solid.

### Step-2: Preparation of (5-bromo-1-(tetrahydrofuran-3-yl)-1H-indazol-3-yl)methanol (96c)

Compound 96c was prepared according to the procedure reported in step-1 of scheme-2 from methyl 5-bromo-1-(tetrahydrofuran-3-yl)-1H-indazole-3-carboxylate (96b) (3.7 g, 11.38 mmol) in DCM (50 mL) using DIBAL (1M solution in DCM, 28.4 mL, 28.4 mmol) to afford after workup and purification using method-F, (5-bromo-1-(tetrahydrofuran-3-yl)-1H-indazol-3-yl)methanol (96c) (3.2 g, 95% yield) as a light yellow oil; 1H NMR (300 MHZ, DMSO-d₆) δ 8.07 (dd, J=1.9, 0.7 Hz, 1H), 7.69 (dd, J=9.0, 0.7 Hz, 1H), 7.51 (dd, J=8.9, 1.9 Hz, 1H), 5.51-5.39 (m, 1H), 5.35 (t, J=5.9 Hz, 1H), 4.75 (d, J=5.7 Hz, 2H), 4.14-3.97 (m, 2H), 3.92-3.79 (m, 2H), 2.48-2.33 (m, 1H), 2.33-2.19 (m, 1H); MS (ES+): 297.00 & 299.00 (M+1); 319.00 & 321.00 (M+Na).

### Step-3: Preparation of ethyl 2-(2-((5-bromo-1-(tetrahydrofuran-3-yl)-1H-indazol-3-yl)methoxy)phenyl)acetate (96d)

Compound 96d was prepared according to the procedure reported in step-2 of scheme-2 from (5-bromo-1-(tetrahydrofuran-3-yl)-1H-indazol-3-yl)methanol (1.6 g, 5.38 mmol) (96c) in DCM (20 mL), using ethyl 2-(2-hydroxyphenyl)acetate (2b) (1.019 g, 5.65 mmol) PPh₃ (1.695 g, 6.46 mmol) and a solution of DCAD (2.373 g, 6.46 mmol) in DCM (20 mL) to afford after workup and purification using method-N, ethyl 2-(2-((5-bromo-1-(tetrahydrofuran-3-yl)-1H-indazol-3-yl)methoxy)phenyl)acetate (96d) (2 g, 81% yield) as a colorless oil; 1H NMR (300 MHz, DMSO-d₆) δ 7.96 (d, J=1.8 Hz, 1H), 7.75 (d, J=9.0 Hz, 1H), 7.56 (dd, J=8.9, 1.8 Hz, 1H), 7.48-7.42 (m, 1H), 7.41-7.36 (m, 1H), 7.23-7.17 (m, 1H), 6.97-6.87 (m, 1H), 5.59-5.43 (m, 1H), 5.37 (s, 2H), 4.12-3.99 (m, 2H), 3.95-3.81 (m, 4H), 3.53 (s, 2H), 2.47-2.37 (m, 1H), 2.37-2.20 (m, 1H), 0.94 (t, J=7.1 Hz, 3H); MS (ES+): 459.00 & 461.00 (M+1); 481.00 & 483.00 (M+Na); (ES−): 457.00 & 459.00 (M−1).

### Step-4: Preparation of ethyl 2-(2-((1-(tetrahydrofuran-3-yl)-5-(4,4,5,5-tetramethyl-1,3,2-dioxaborolan-2-yl)-1H-indazol-3-yl)methoxy)phenyl)acetate (96e)

Compound 96e was prepared according to the procedure reported in step-4 of scheme-1, from ethyl 2-(2-((5-bromo-1-(tetrahydrofuran-3-yl)-1H-indazol-3-yl)methoxy)phenyl)acetate (96d) (1.1 g, 2.395 mmol) in anhydrous dioxane (30 mL), using BISPIN (1.216 g, 4.79 mmol), KOAc (0.588 g, 5.99 mmol) PdCl₂(dppf)-CH₂Cl₂ adduct (0.117 g, 0.144 mmol) to afford after workup and purification using method-AA, ethyl 2-(2-((1-(tetrahydrofuran-3-yl)-5-(4,4,5,5-tetramethyl-1,3,2-dioxaborolan-2-yl)-1H-indazol-3-yl)methoxy)phenyl)acetate (96e) (1.1 g, 91% yield) as a clear oil; MS (ES+): 507.25 (M+1).

### Step-5: Preparation of ethyl 2-(2-((5-(3-carbamimidoylphenyl)-1-(tetrahydrofuran-3-yl)-1H-indazol-3-yl)methoxy)phenyl)acetate (96f)

Compound 96f was prepared according to the procedure reported in step-5 of scheme-1, from ethyl 2-(2-((1-(tetrahydrofuran-3-yl)-5-(4,4,5,5-tetramethyl-1,3,2-dioxaborolan-2-yl)-1H-indazol-3-yl)methoxy)phenyl)acetate (96e) (300 mg, 0.592 mmol) in dioxane/THF (4 mL, 1:1) using 3-bromobenzimidamide hydrochloride (1g) (279 mg, 1.185 mmol), K₃PO₄ (2M aqueous solution, 1.185 mL, 2.370 mmol), PCy₃ (33.2 mg, 0.118 mmol), PdCl₂(dppf)-CH₂Cl₂ adduct (48.4 mg, 0.059 mmol) and Pd₂(dba)₃ (54.2 mg, 0.059 mmol) to afford after workup and purification using method-AI, ethyl 2-(2-((5-(3-carbamimidoylphenyl)-1-(tetrahydrofuran-3-yl)-1H-indazol-3-yl)methoxy)phenyl)acetate (96f) (160 mg, 54.2% yield) as a clear oil; MS (ES+): 499.25 (M+1).

### Step-6: Preparation of 2-(2-((5-(3-carbamimidoylphenyl)-1-(tetrahydrofuran-3-yl)-1H-indazol-3-yl)methoxy)phenyl)acetic acid (96g)

Compound 96g was prepared according to the procedure reported in step-6 of scheme-1, from ethyl 2-(2-((5-(3-carbamimidoylphenyl)-1-(tetrahydrofuran-3-yl)-1H-indazol-3-yl)methoxy)phenyl)acetate (96f) (160 mg, 0.321 mmol) in MeOH/THF (6 mL, 1:1) using a solution of LiOH·H₂O (81 mg, 1.925 mmol) in water (2 mL) to afford after workup and purification using method-G, 2-(2-((5-(3-carbamimidoylphenyl)-1-(tetrahydrofuran-3-yl)-1H-indazol-3-yl)methoxy)phenyl)acetic acid (96g) (30 mg, 19.87% yield) HCl salt as a white solid; ¹H NMR (300 MHz, DMSO-d₆) δ 12.09 (s, 1H, D₂O exchangeable), 9.45 (s, 2H, D₂O exchangeable), 9.17 (s, 2H, D₂O exchangeable), 8.24-8.13 (m, 2H), 8.10 (dt, 1H), 7.96-7.86 (m, 2H), 7.85-7.75 (m, 1H), 7.70 (t, J=7.7 Hz, 1H), 7.32-7.23 (m, 2H), 7.23-7.16 (m, 1H), 6.99-6.86 (m, 1H), 5.62-5.50 (m, 1H), 5.46 (s, 2H), 4.20-4.04 (m, 2H), 4.01-3.85 (m, 2H), 3.52 (s, 2H), 2.49-2.26 (m, 2H); MS (ES+): 471.20 (M+1); (ES−): 469.10 (M−1).

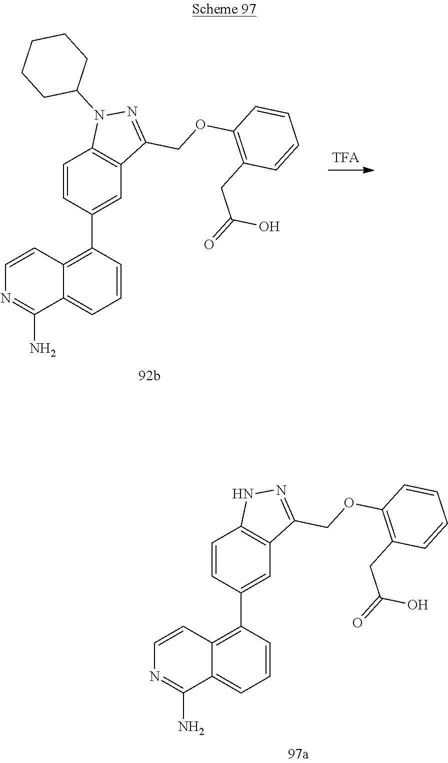 [SMILES: Nc1nccc2c(-c3ccc4c(c3)c(COc3ccccc3CC(=O)O)nn4C3CCCCC3)cccc12 ; Nc1nccc2c(-c3ccc4[nH]nc(COc5ccccc5CC(=O)O)c4c3)cccc12]

### Preparation of 2-(2-((5-(1-aminoisoquinolin-5-yl)-1H-indazol-3-yl)methoxy)phenyl)acetic acid (97a)

Compound 97a was prepared according to the procedure reported in step-4 of scheme-9 from 2-(2-((5-(1-aminoisoquinolin-5-yl)-1-(tetrahydro-2H-pyran-2-yl)-1H-indazol-3-yl)methoxy)phenyl)acetic acid (92b) (120 mg, 0.236 mmol) in DCM (6 mL) using TFA (0.364 mL, 4.72 mmol) to afford after workup and purification using method-G, 2-(2-((5-(1-aminoisoquinolin-5-yl)-1H-indazol-3-yl)methoxy)phenyl)acetic acid (97a) (50 mg, 49.9% yield) HCl salt as a white solid; 1H NMR (300 MHz, DMSO-d₆) δ 13.33 (s, 1H, D₂O exchangeable), 13.24 (s, 1H, D₂O exchangeable), 12.04 (s, 1H, D₂O exchangeable), 9.17 (s, 2H, D₂O exchangeable), 8.61 (d, J=8.3 Hz, 1H), 8.00-7.91 (m, 1H), 7.91-7.80 (m, 2H), 7.74-7.67 (m, 1H), 7.62 (d, J=7.3 Hz, 1H), 7.44 (dd, J=8.6, 1.6 Hz, 1H), 7.29-7.20 (m, 2H), 7.20-7.13 (m, 1H), 6.99 (d, J=7.2 Hz, 1H), 6.95-6.84 (m, 1H), 5.46 (s, 2H), 3.48 (s, 2H); MS (ES+): 425.20 (M+1).

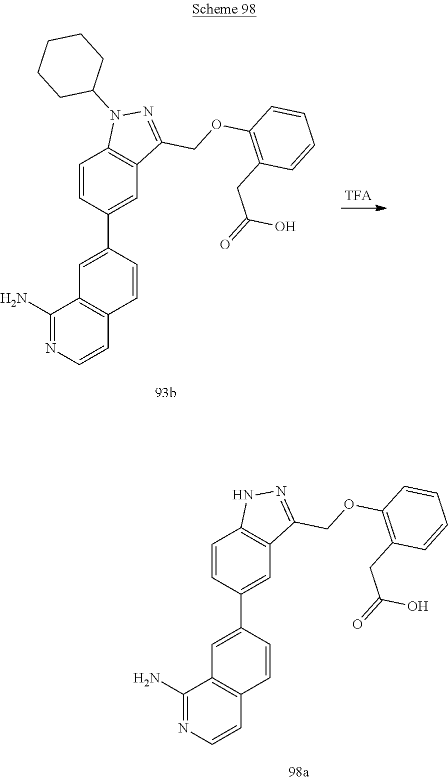 [SMILES: Nc1nccc2ccc(-c3ccc4c(c3)c(COc3ccccc3CC(=O)O)nn4C3CCCCC3)cc12 ; Nc1nccc2ccc(-c3ccc4[nH]nc(COc5ccccc5CC(=O)O)c4c3)cc12]

### Preparation of 2-(2-((5-(1-aminoisoquinolin-7-yl)-1H-indazol-3-yl)methoxy)phenyl)acetic acid (98a)

Compound 98a was prepared according to the procedure reported in step-4 of scheme-9 from 2-(2-((5-(1-aminoisoquinolin-7-yl)-1-(tetrahydro-2H-pyran-2-yl)-1H-indazol-3-yl)methoxy)phenyl)acetic acid (93b) (120 mg, 0.236 mmol) in DCM (6 mL) using TFA (0.364 mL, 4.72 mmol) to afford after workup and purification using method-G, 2-(2-((5-(1-aminoisoquinolin-7-yl)-1H-indazol-3-yl)methoxy)phenyl)acetic acid (98a) (45 mg, 44.9% yield) HCl salt as a white solid; 1H NMR (300 MHz, DMSO-d₆) δ 13.47 (s, 1H, D₂O exchangeable), 13.33 (s, 1H, D₂O exchangeable), 9.25 (s, 2H, D₂O exchangeable), 9.03-8.89 (m, 1H), 8.40 (dd, 1H), 8.36-8.24 (m, 1H), 8.04 (d, J=8.5 Hz, 1H), 8.01-7.89 (m, 1H), 7.77-7.61 (m, 2H), 7.34-7.23 (m, 3H), 7.23-7.14 (m, 1H), 6.97-6.84 (m, 1H), 5.50 (s, 2H), 3.53 (s, 2H); MS (ES+): 425.10 (M+1); Analysis calculated for C₂₅H₂₀N₄O₃·HCl·1.75H₂O: C, 60.98; H, 5.01; Cl, 7.20; N, 11.38; Found: C, 61.10; H, 5.00; Cl, 7.38; N, 11.16.

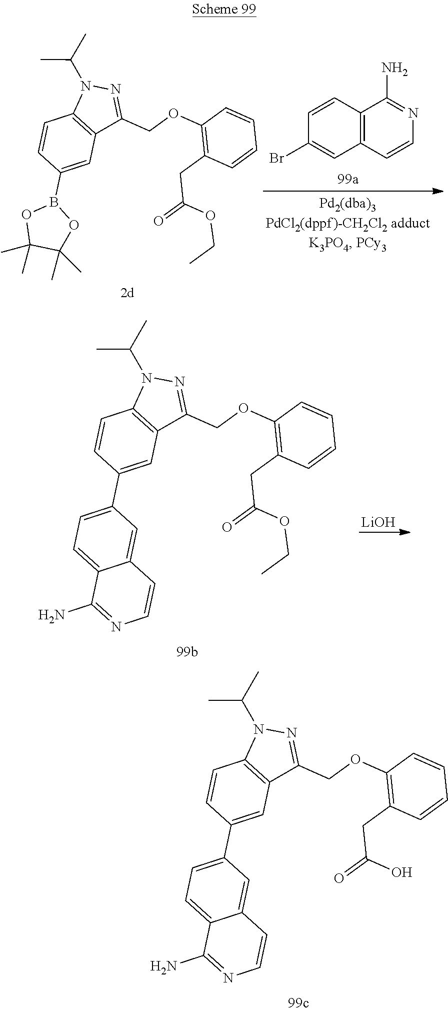

### Preparation of 2-(2-((5-(1-aminoisoquinolin-6-yl)-1-isopropyl-1H-indazol-3-yl)methoxy)phenyl)acetic acid (99c)

### Step-1: Preparation of ethyl 2-(2-((5-(1-aminoisoquinolin-6-yl)-1-isopropyl-1H-indazol-3-yl)methoxy)phenyl)acetate (99b)

Compound 99b was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 6-bromoisoquinolin-1-amine (99a) (187 mg, 0.836 mmol; CAS #215453-26-2), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(1-aminoisoquinolin-6-yl)-1-isopropyl-1H-indazol-3-yl)methoxy)phenyl)acetate (99b) (150 mg, 72.5% yield) as a clear oil; 1H NMR (300 MHz, DMSO-d₆) δ 8.28 (d, J=8.7 Hz, 1H), 8.13 (d, J=8.2 Hz, 2H), 8.02 (s, 1H), 7.97 (d, J=2.0 Hz, 1H), 7.87 (d, J=2.5 Hz, 2H), 7.80 (d, J=6.9 Hz, 1H), 7.59 (dd, J=8.9, 2.1 Hz, 1H), 7.30 (d, J=4.1 Hz, 2H), 7.20 (d, J=7.3 Hz, 1H), 5.45 (s, 2H), 5.15-4.95 (m, 1H), 4.11 (q, J=5.3 Hz, 2H), 3.54 (s, 2H), 1.52 (d, J=6.6 Hz, 6H), 0.72 (t, J=7.1 Hz, 3H); MS (ES+): 495.25 (M+1); (ES−): 493.25 (M−1).

### Step-2: Preparation of 2-(2-((5-(1-aminoisoquinolin-6-yl)-1-isopropyl-1H-indazol-3-yl)methoxy)phenyl)acetic acid (99c)

Compound 99c was prepared according to the procedure reported in step-6 of scheme-1, from ethyl 2-(2-((5-(1-aminoisoquinolin-6-yl)-1-isopropyl-1H-indazol-3-yl)methoxy)phenyl)acetate (99b) (150 mg, 0.303 mmol) in THF/MeOH (4 mL, 1:1) using a solution of LiOH·H₂O (76 mg, 1.820 mmol) in water (1 mL) to afford after workup and purification using method-G, 2-(2-((5-(1-aminoisoquinolin-6-yl)-1-isopropyl-1H-indazol-3-yl)methoxy)phenyl)acetic acid (99c) (60 mg, 42.4% yield) HCl salt as a white solid; ¹H NMR (300 MHz, DMSO-d₆) δ 13.29 (s, 1H, D₂O exchangeable), 12.10 (s, 1H, D₂O exchangeable), 9.14 (s, 2H, D₂O exchangeable), 8.67 (d, J=8.8 Hz, 1H), 8.37-8.28 (m, 2H), 8.20 (dd, J=8.8, 1.9 Hz, 1H), 8.00-7.85 (m, 2H), 7.71 (d, J=7.0 Hz, 1H), 7.35-7.16 (m, 4H), 6.93 (ddd, J=8.0, 6.0, 2.5 Hz, 1H), 5.49 (s, 2H), 5.17-4.94 (m, 1H), 3.55 (s, 2H), 1.53 (d, J=6.6 Hz, 6H); MS (ES+): 467.20 (M+1); (ES−): 465.20 (M−1).

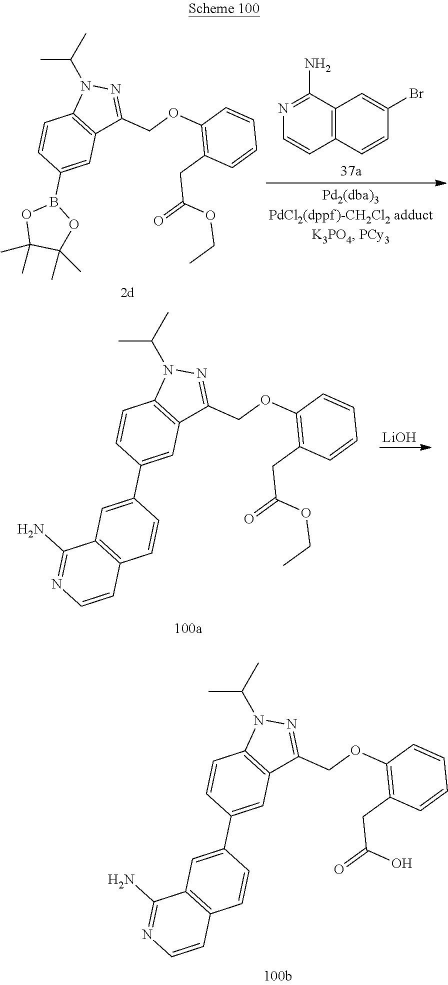

### Preparation of 2-(2-((5-(1-aminoisoquinolin-7-yl)-I-isopropyl-1H-indazol-3-yl)methoxy)phenyl)acetic acid (100b)

### Step-1: Preparation of ethyl 2-(2-((5-(1-aminoisoquinolin-7-yl)-1-isopropyl-1H-indazol-3-yl)methoxy)phenyl)acetate (100a)

Compound 100a was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 7-bromoisoquinolin-1-amine (37a) (187 mg, 0.836 mmol), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(1-aminoisoquinolin-7-yl)-1-isopropyl-1H-indazol-3-yl)methoxy)phenyl)acetate (100a) (150 mg, 72.5% yield) as a clear oil; MS (ES+): 495.20 (M+1); (ES−): 493.05 (M−1).

### Step-2: Preparation of 2-(2-((5-(1-aminoisoquinolin-7-yl)-1-isopropyl-1H-indazol-3-yl)methoxy)phenyl)acetic acid (100b)

Compound 100b was prepared according to the procedure reported in step-6 of scheme-1, from ethyl 2-(2-((5-(1-aminoisoquinolin-7-yl)-1-isopropyl-1H-indazol-3-yl)methoxy)phenyl)acetate (100a) (150 mg, 0.303 mmol) in THF/MeOH (4 mL, 1:1) using a solution of LiOH·H₂O (76 mg, 1.820 mmol) in water (1 mL) to afford after workup and purification using method-G, 2-(2-((5-(1-aminoisoquinolin-7-yl)-1-isopropyl-1H-indazol-3-yl)methoxy)phenyl)acetic acid (100b) (35 mg, 24.74% yield) HCl salt as a white solid; ¹H NMR (300 MHz, DMSO-d₆) δ 13.45 (s, 1H, D₂O exchangeable), 9.24 (s, 2H, D₂O) exchangeable), 8.98 (s, 1H), 8.41 (dd, J=8.5, 1.6 Hz, 1H), 8.31 (s, 1H), 8.09-7.96 (m, 2H), 7.91 (d, J=8.9 Hz, 1H), 7.69 (d, J=6.9 Hz, 1H), 7.34-7.16 (m, 4H), 7.00-6.85 (m, 1H), 5.48 (s, 2H), 5.18-4.96 (m, 1H), 3.54 (s, 2H), 1.53 (d, J=6.5 Hz, 6H); MS (ES+): 467.20 (M+1); (ES−): 465.20 (M−1); Analysis calculated for C₂₈H₂₆N₄O₃·HCl·1.75H₂O: C, 62.92; H, 5.75; N, 10.48; Found: C, 62.83; H, 5.54; N, 10.52.

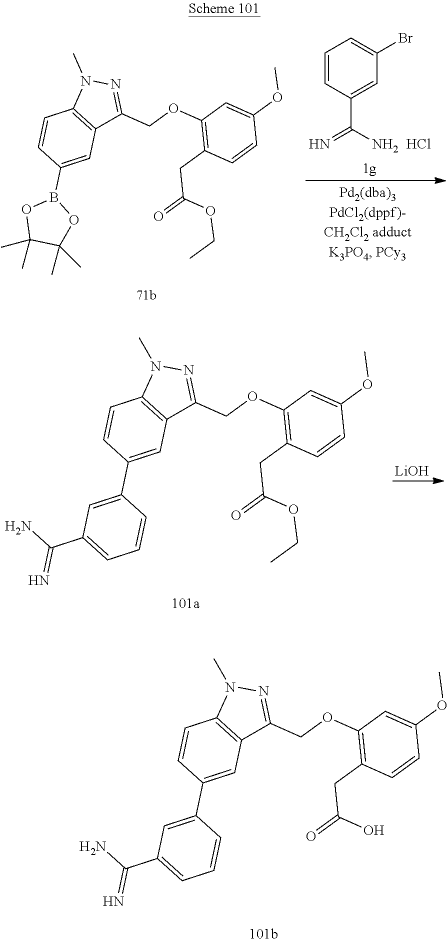 [SMILES: CCOC(=O)Cc1ccc(OC)cc1OCc1nn(C)c2ccc(B3OC(C)(C)C(C)(C)O3)cc12 ; N=C(N)c1cccc(Br)c1 ; Cl ; CCOC(=O)Cc1ccc(OC)cc1OCc1nn(C)c2ccc(-c3cccc(C(=N)N)c3)cc12 ; "LiOH" ; COc1ccc(CC(=O)O)c(OCc2nn(C)c3ccc(-c4cccc(C(=N)N)c4)cc23)c1]

### Preparation of 2-(2-((5-(3-carbamimidoylphenyl)-1-methyl-1H-indazol-3-yl)methoxy)-4-methoxyphenyl)acetic acid (101b)

### Step-1: Preparation of ethyl 2-(2-((5-(3-carbamimidoylphenyl)-1-methyl-1H-indazol-3-yl)methoxy)-4-methoxyphenyl)acetate (101a)

Compound 101a was prepared according to the procedure reported in step-5 of scheme-1, from ethyl 2-(4-methoxy-2-((1-methyl-5-(4,4,5,5-tetramethyl-1,3,2-dioxaborolan-2-yl)-1H-indazol-3-yl)methoxy)phenyl)acetate (71b) (500 mg, 1.041 mmol) in dioxane/THF (6 mL, 1:1) using 3-bromobenzimidamide hydrochloride (1g) (490 mg, 2.082 mmol), K₃PO₄ (2M aqueous solution, 2.082 mL, 4.16 mmol), PCy₃ (58.4 mg, 0.208 mmol), PdCl₂(dppf)-CH₂Cl₂ adduct (0.085 g, 0.104 mmol) and Pd₂(dba)₃ (95 mg, 0.104 mmol) to afford after workup and purification using method-E, ethyl 2-(2-(5-(3-carbamimidoylphenyl)-1-methyl-1H-indazol-3-yl)methoxy)-4-methoxyphenyl)acetate (101a) (160 mg, 32.5% yield) as a clear oil; ¹H NMR (300 MHz, DMSO-d₆) δ 8.97 (d, J=101.9 Hz, 3H), 8.12 (d, J=4.3 Hz, 2H), 8.01 (d, J=7.8 Hz, 1H), 7.88 (d, J=8.9 Hz, 1H), 7.83-7.71 (m, 2H), 7.64 (t, J=7.7 Hz, 1H), 7.08 (d, J=8.3 Hz, 1H), 6.87 (d, J=2.4 Hz, 1H), 6.49 (dd, J=8.3, 2.4 Hz, 1H), 5.42 (s, 2H), 4.09 (s, 3H), 3.76 (s, 3H), 3.66 (q, J=7.1 Hz, 2H), 3.43 (s, 2H), 0.79 (t, J=7.1 Hz, 3H); MS (ES+): 473.20 (M+1); (ES−): 507.10 (M+CI).

### Step-2: Preparation of 2-(2-((5-(3-carbamimidoylphenyl)-1-methyl-1H-indazol-3-yl)methoxy)-4-methoxyphenyl)acetic acid (101b)

Compound 101b was prepared according to the procedure reported in step-6 of scheme-1, from ethyl 2-(2-((5-(3-carbamimidoylphenyl)-1-methyl-1H-indazol-3-yl)methoxy)-4-methoxyphenyl)acetate (101a) (160 mg, 0.339 mmol) in THE/MeOH (4 mL) using a solution of LiOH·H₂O (85 mg, 2.032 mmol) in water (1 mL) to afford after workup and purification using method-G, 2-(2-((5-(3-carbamimidoylphenyl)-1-methyl-1H-indazol-3-yl)methoxy)-4-methoxyphenyl)acetic acid (101b) (50 mg, 33.2% yield) HCl salt as a white solid; ¹H NMR (300 MHz, DMSO-d₆) δ 11.98 (s, 1H, D₂O exchangeable), 9.43 (s, 2H, D₂O exchangeable), 9.12 (s, 2H, D₂O exchangeable), 8.21 (s, 1H), 8.15 (s, 1H), 8.10 (d, J=7.6 Hz, 1H), 7.90 (dd, J=8.9, 1.7 Hz, 1H), 7.85-7.74 (m, 2H), 7.70 (t, J=7.7 Hz, 1H), 7.08 (d, J=8.3 Hz, 1H), 6.87 (d, J=2.5 Hz, 1H), 6.49 (dd, J=8.3, 2.3 Hz, 1H), 5.44 (s, 2H), 4.10 (s, 3H), 3.75 (s, 3H), 3.42 (s, 2H); MS (ES+): 445.20 (M+1); (ES−): 443.15 (M−1); Analysis calculated for C₂₅H₂₄N₄O₄·1.2HCl·1.5H₂O: C, 58.28; H, 5.52; Cl, 8.26; N, 10.87; Found: C, 58.49; H, 5.52; Cl, 7.98; N, 10.75.

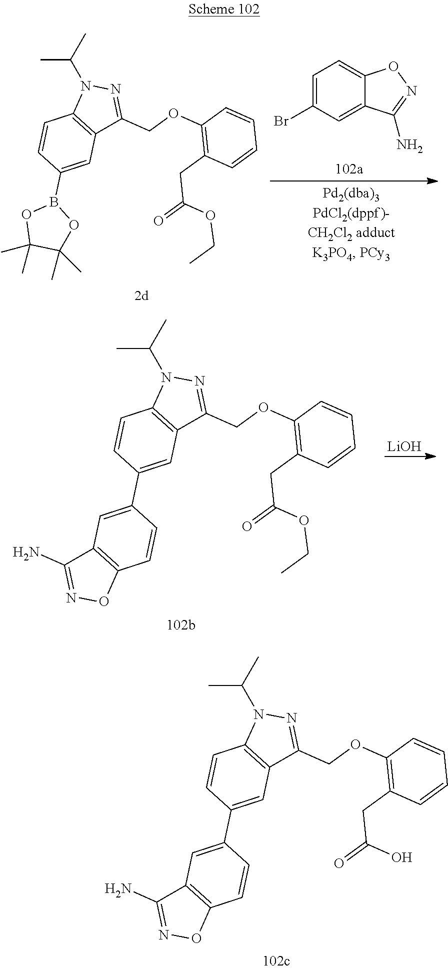

### Preparation of 2-(2-((5-(3-aminobenzo[d]isoxazol-5-yl)-1-isopropyl-1H-indazol-3-yl)methoxy)phenyl)acetic acid (102c)

### Step-1: Preparation of ethyl 2-(2-((5-(3-aminobenzo[d]isoxazol-5-yl)-1-isopropyl-1H-indazol-3-yl)methoxy)phenyl)acetate (102b)

Compound 102b was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, Ratio: 1:1) using 5-bromobenzo[d]isoxazol-3-amine (102a) (178 mg, 0.836 mmol; CAS #455280-00-9), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-E, ethyl 2-(2-((5-(3-aminobenzo[d]isoxazol-5-yl)-1-isopropyl-1H-indazol-3-yl)methoxy)phenyl)acetate (102b) (50 mg, 24.68% yield) as a clear oil; ¹H NMR (300 MHz, DMSO-d₆) δ 8.15 (d, J=1.8 Hz, 1H), 7.99 (d, J=1.6 Hz, 1H), 7.91-7.80 (m, 2H), 7.72 (dd, J=8.8, 1.7 Hz, 1H), 7.52 (d, J=8.7 Hz, 1H), 7.32-7.25 (m, 2H), 7.22-7.16 (m, 1H), 6.96-6.88 (m, 1H), 5.42 (s, 2H), 5.04 (p, J=6.6 Hz, 1H), 3.60 (q, J=7.1 Hz, 2H), 3.52 (s, 2H), 1.52 (d, J=6.6 Hz, 6H), 0.73 (t, J=7.1 Hz, 3H); MS (ES+): 485.20 (M+1); 507.10 (M+Na); (ES−): 483.10 (M−1).

### Step-2: Preparation of 2-(2-((5-(3-aminobenzo[d]isoxazol-5-yl)-1-isopropyl-1H-indazol-3-yl)methoxy)phenyl)acetic acid (102c)

Compound 102c was prepared according to the procedure reported in step-5 of scheme-2 from ethyl 2-(2-((5-(3-aminobenzo[d]isoxazol-5-yl)-1-isopropyl-1H-indazol-3-yl)methoxy)phenyl)acetate (102b) (80 mg, 0.165 mmol) in THF/MeOH (4 mL, 1:1) using a solution of LiOH·H₂O (41.6 mg, 0.991 mmol) in water (I mL) and heating at 50° C. for 1 h. This gave after workup and purification using method-G, 2-(2-((5-(3-aminobenzo[d]isoxazol-5-yl)-1-isopropyl-1H-indazol-3-yl)methoxy)phenyl)acetic acid (102c) (55 mg, 73.0% yield) HCl salt as a white solid; ¹H NMR (300 MHz, DMSO-d₆) δ 8.16 (d, J=1.8 Hz, 1H), 8.04 (d, J=1.6 Hz, 1H), 7.91-7.80 (m, 2H), 7.72 (dd, J=8.8, 1.7 Hz, 1H), 7.52 (d, J=8.7 Hz, 1H), 7.32-7.23 (m, 2H), 7.23-7.16 (m, 1H), 6.96-6.85 (m, 1H), 5.45 (s, 2H), 5.10-4.97 (m, 1H), 3.52 (s, 2H), 1.52 (d, J=6.6 Hz, 6H); MS (ES+): 457.20 (M+1); (ES−): 455.15 (M−1).

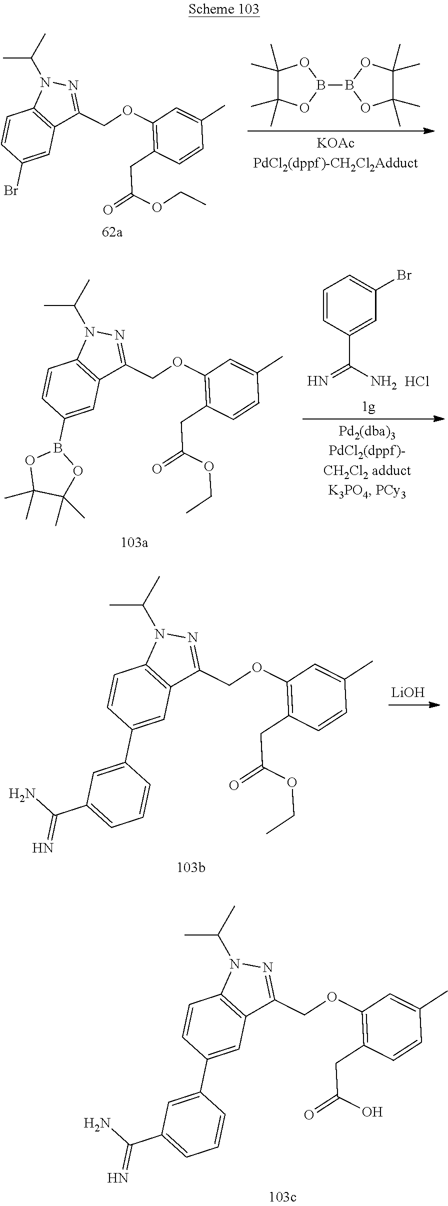

### Preparation of 2-(2-((5-(3-carbamimidoylphenyl)-1-isopropyl-1H-indazol-3-yl)methoxy)-4-methylphenyl)acetic acid (103c)

### Step-1: Preparation of ethyl 2-(2-((1-isopropyl-5-(4,4,5,5-tetramethyl-1,3,2-dioxaborolan-2-yl)-1H-indazol-3-yl)methoxy)-4-methylphenyl)acetate (103a)

Compound 103a was prepared according to the procedure reported in step-4 of scheme-1, from ethyl 2-(2-((5-bromo-1-isopropyl-1H-indazol-3-yl)methoxy)-4-methylphenyl)acetate (62a) (600 mg, 1.347 mmol) in anhydrous dioxane (20 mL), using BISPIN (684 mg, 2.69 mmol), KOAc (331 mg, 3.37 mmol) and PdCl₂(dppf)-CH₂Cl₂ adduct (66.0 mg, 0.081 mmol) to afford after workup and purification using method-AA, ethyl 2-(2-((1-isopropyl-5-(4,4,5,5-tetramethyl-1,3,2-dioxaborolan-2-yl)-1H-indazol-3-yl)methoxy)-4-methylphenyl)acetate (103a) (560 mg, 84% yield) as a clear oil; 1H NMR (300 MHz, DMSO-d₆) δ 8.15 (d, J=1.0 Hz, 1H), 7.13 (s, 1H), 7.04 (d, J=7.5 Hz, 1H), 6.95 (d, J=7.6 Hz, 1H), 6.72 (d, J=7.4 Hz, 1H), 6.59 (s, 1H), 5.38 (s, 2H), 5.00 (p, J=6.6 Hz, 1H), 3.87 (q, J=7.1 Hz, 2H), 3.45 (s, 2H), 2.31 (s, 3H), 1.48 (d, J=6.6 Hz, 6H), 1.29 (s, 12H), 0.90 (t, J=7.1 Hz, 3H); MS (ES+): 493.30 (M+1); 515.30 (M+Na).

### Step-2: Preparation of ethyl 2-(2-((5-(3-carbamimidoylphenyl)-1-isopropyl-1H-indazol-3-yl)methoxy)-4-methylphenyl)acetate (103b)

Compound 103b was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)-4-methylphenyl)acetate (103a) (560 mg, 1.137 mmol) in dioxane/THF (6 mL. Ratio: 1:1) using 3-bromobenzimidamide hydrochloride (1g) (536 mg, 2.275 mmol). K₃PO₄ (2M aqueous solution, 2.275 mL, 4.55 mmol), PCy₃ (63.8 mg, 0.227 mmol), PdCl₂(dppf)-CH₂Cl₂ adduct (0.093 g, 0.114 mmol) and Pd₂(dba)₃ (104 mg, 0.114 mmol) to afford after workup and purification using method-F, ethyl 2-(2-((5-(3-carbamimidoylphenyl)-1-isopropyl-1H-indazol-3-yl)methoxy)-4-methylphenyl)acetate (103b) (170 mg, 30.8% yield) as a clear oil; ¹H NMR (300 MHz, DMSO-d₆) δ 9.86 (m, 2H), 8.12 (s, 2H), 8.07 (d, J=7.4 Hz, 1H), 7.85 (s, 2H), 7.70 (d, J=7.5 Hz, 1H), 7.13 (s, 1H), 7.04 (d, J=7.5 Hz, 1H), 6.82 (d, J=7.5 Hz, 1H), 6.72 (d, J=7.4 Hz, 1H), 6.55-6.40 (m, 2H), 5.42 (s, 2H), 5.06 (p, J=6.5 Hz, 1H), 3.66 (q. J=7.0 Hz, 2H), 3.48 (s, 2H), 2.31 (s, 3H), 1.52 (d, J=6.4 Hz, 6H), 0.78 (t, J=7.0 Hz, 3H); MS (ES+): 485.30 (M+1).

### Step-3: Preparation of 2-(2-((5-(3-carbamimidoylphenyl)-1-isopropyl-1H-indazol-3-yl)methoxy)-4-methylphenyl)acetic acid (103c)

Compound 103c was prepared according to the procedure reported in step-5 of scheme-2, from ethyl 2-(2-((5-(3-carbamimidoylphenyl)-1-isopropyl-1H-indazol-3-yl)methoxy)-4-methylphenyl)acetate (103b) (170 mg, 0.351 mmol) in THF/MeOH (4 mL) using a solution of LiOH. H₂O (88 mg, 2.105 mmol) in water (2 mL) to afford after workup and purification using method-G, 2-(2-((5-(3-carbamimidoylphenyl)-1-isopropyl-1H-indazol-3-yl)methoxy)-4-methylphenyl)acetic acid (103c) (55 mg, 34.3% yield) HCl salt as a white solid; ¹H NMR (300 MHz, DMSO-d₆) δ 12.03 (s, 1H, D₂O exchangeable), 9.43 (s, 2H, D₂O exchangeable), 9.13 (s, 2H, D₂O exchangeable), 8.20-8.03 (m, 3H), 7.87 (s, 2H), 7.81-7.62 (m, 2H), 7.14 (s, 1H), 7.05 (d, J=7.5 Hz, 1H), 6.72 (d, J=7.5 Hz, 1H), 5.45 (s, 2H), 5.23-4.83 (m, 1H), 3.47 (s, 2H), 2.29 (s, 3H), 1.52 (d, J=6.5 Hz, 6H); MS (ES+): 457.25 (M+1); MS (ES−): 455.15 (M−1); Analysis calculated for C₂₇H₂₈N₄O₃·1.2HCl·H₂O: C, 62.57; H, 6.07; Cl, 8.21; N, 10.81; Found: C, 62.61; H, 5.96; Cl, 7.80; N, 10.82.

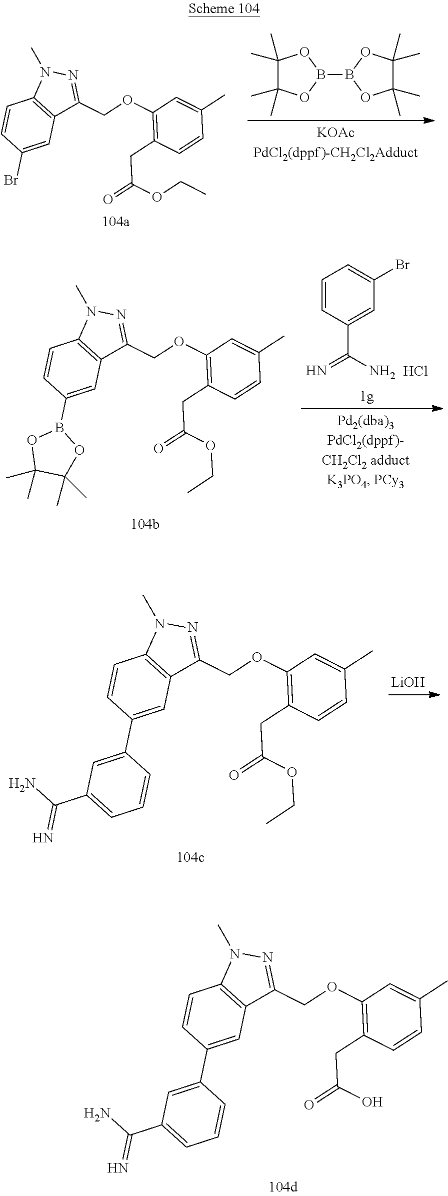

### Preparation of 2-(2-((5-(3-carbamimidoylphenyl)-1-methyl-1H-indazol-3-yl)methoxy)-4-methylphenyl)acetic acid (104d)

### Step-1: Preparation of ethyl 2-(4-methyl-2-((1-methyl-5-(4,4,5,5-tetramethyl-1,3,2-dioxaborolan-2-yl)-1H-indazol-3-yl)methoxy)phenyl)acetate (104b)

Compound 104b was prepared according to the procedure reported in step-4 of scheme-1, from ethyl 2-(2-((5-bromo-1-methyl-1H-indazol-3-yl)methoxy)-4-methylphenyl)acetate (104a) (400 mg, 0.959 mmol) in anhydrous dioxane (20 mL), using BISPIN (487 mg, 1.917 mmol), KOAc (235 mg, 2.396 mmol) and PdCl₂(dppf)-CH₂Cl₂ adduct (47.0 mg, 0.058 mmol) to afford after workup and purification using method-C, ethyl 2-(4-methyl-2-((1-methyl-5-(4,4,5,5-tetramethyl-1,3,2-dioxaborolan-2-yl)-1H-indazol-3-yl)methoxy)phenyl)acetate (104b) (400 mg, 90% yield) as a clear oil; ¹H NMR (300 MHz, DMSO-d₆) δ 8.16 (d, J=1.0 Hz, 1H), 7.71-7.60 (m, 2H), 7.13 (s, 1H), 7.06 (d, J=7.5 Hz, 1H), 6.74 (d, J=7.4 Hz, 1H), 5.37 (s, 2H), 4.06 (s, 3H), 3.84 (q, J=7.1 Hz, 2H), 3.44 (s, 2H), 2.33 (s, 3H), 1.30 (s, 12H), 0.88 (t, J=7.1 Hz, 3H); MS (ES+): 465.30 (M+1); (ES−): 463.15 (M−1).

### Step-2: Preparation of ethyl 2-(2-((5-(3-carbamimidoylphenyl)-1-methyl-1H-indazol-3-yl)methoxy)-4-methylphenyl)acetate (104c)

prepared according to the procedure reported in step-5 of scheme-1, from ethyl 2-(4-methyl-2-((1-methyl-5-(4,4,5,5-tetramethyl-1,3,2-dioxaborolan-2-yl)-1H-indazol-3-yl)methoxy)phenyl)acetate (104b) (400 mg, 0.861 mmol) in dioxane/THF (6 mL, Ratio: 1:1) using 3-bromobenzimidamide hydrochloride (1g) (406 mg, 0.086 mmol), K₃PO₄ (2M aqueous solution, 1.723 mL, 3.45 mmol), PCy₃ (48.3 mg, 0.172 mmol), PdCl₂(dppf)-CH₂Cl₂ adduct (0.0703 g, 0.114 mmol) and Pd₂(dba)₃ (79 mg, 0.086 mmol) to afford after workup and purification using method-F, ethyl 2-(2-((5-(3-carbamimidoylphenyl)-1-methyl-1H-indazol-3-yl)methoxy)-4-methylphenyl)acetate (104c) (130 mg, 33.1% yield) as a clear oil; 1H NMR (300 MHz, DMSO-d₆)>9.17 (s, 3H), 8.14 (d, J=5.5 Hz, 2H), 8.06 (d, J=7.5 Hz, 1H), 7.89 (d, J=8.7 Hz, 1H), 7.84-7.72 (m, 2H), 7.68 (t, J=7.5 Hz, 1H), 7.60-7.32 (m, 2H), 7.13 (s, 1H), 7.05 (d, J=7.6 Hz, 1H), 6.73 (d, J=7.6 Hz, 1H), 5.40 (s, 2H), 4.10 (s, 3H), 3.65 (q, J=7.0 Hz, 2H), 3.46 (s, 2H), 2.32 (s, 3H), 0.78 (t, J=7.1 Hz, 3H); MS (ES+): 457.20 (M+1).

### Step-3: Preparation of 2-(2-((5-(3-carbamimidoylphenyl)-1-methyl-1H-indazol-3-yl)methoxy)-4-methylphenyl)acetic acid (104d)

Compound 104d was prepared according to the procedure reported in step-5 of scheme-2, from ethyl 2-(2-((5-(3-carbamimidoylphenyl)-1-methyl-1H-indazol-3-yl)methoxy)-4-methylphenyl)acetate (104c) (130 mg, 0.285 mmol) in THE/MeOH (4 mL, 1:1) using a solution of LiOH·H₂O (71.7 mg, 1.709 mmol) in water (1 mL) to afford after workup and purification using method-G, 2-(2-((5-(3-carbamimidoylphenyl)-1-methyl-1H-indazol-3-yl)methoxy)-4-methylphenyl)acetic acid (104d) (55 mg, 45.1% yield) HCl salt as a white solid; 1H NMR (300 MHz, DMSO-d₆) δ 12.01 (s, 1H, D₂O exchangeable), 9.42 (s, 2H, D₂O exchangeable), 9.09 (s, 2H, D₂O exchangeable), 8.20 (s, 1H), 8.16-8.06 (m, 2H), 7.90 (dd, J=8.9, 1.6 Hz, 1H), 7.84-7.74 (m, 2H), 7.70 (t, J=7.6 Hz, 1H), 7.13 (s, 1H), 7.06 (d, J=7.6 Hz, 1H), 6.74 (d, J=7.8 Hz, 1H), 5.41 (s, 2H), 4.10 (s, 3H), 3.44 (s, 2H), 2.31 (s, 3H); MS (ES+): 429.20 (M+1); (ES−): 427.20 (M−1); Analysis calculated for C₂₅H₂₄N₄O₃·1.1HCl·2H₂O: C, 59.50; H, 5.81; Cl, 7.73; N, 11.10; Found: C, 59.49; H, 5.47; Cl, 7.96; N, 11.22.

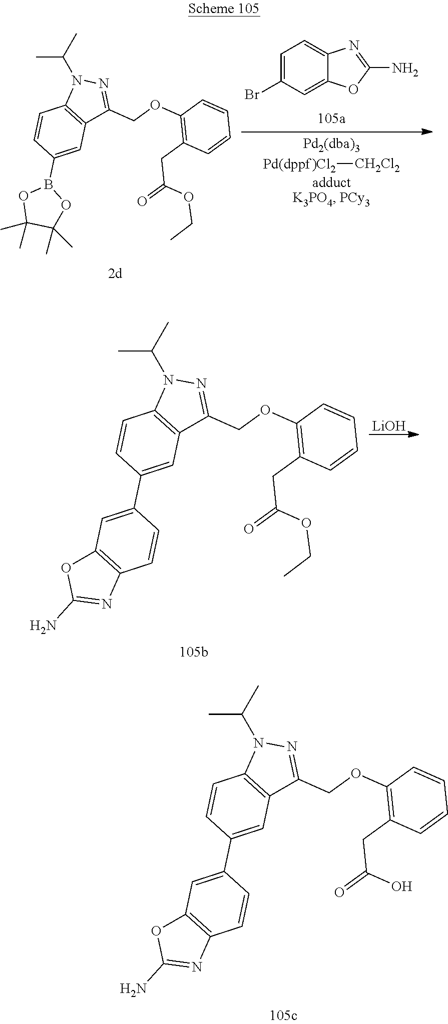

### Preparation of 2-(2-((5-(2-aminobenzo[d]oxazol-6-yl)-1-isopropyl-1H-indazol-3-yl)methoxy)phenyl)acetic acid (105c)

### Step-1: Preparation of ethyl 2-(2-((5-(2-aminobenzo[d]oxazol-6-yl)-I-isopropyl-1H-indazol-3-yl)methoxy)phenyl)acetate (105b)

Compound 105b was prepared according to the procedure reported in step-5 of scheme-1, from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 6-bromobenzo[d]oxazol-2-amine (105a) (178 mg, 0.836 mmol; CAS #52112-66-0), K₃PO₄ (2M aqueous solution, 0.836 mL, 1.672 mmol), PCy₃ (23.45 mg, 0.084 mmol), Pd₂(dba)₃ (38.3 mg, 0.042 mmol) and PdCl₂(dppf)-CH₂Cl₂ adduct (34.1 mg, 0.042 mmol) to afford after workup and purification using method-U, ethyl 2-(2-((5-(2-aminobenzo[d]oxazol-6-yl)-1-isopropyl-1H-indazol-3-yl)methoxy)phenyl)acetate (105b) (140 mg, 69.1% yield) as a clear oil; 1H NMR (300 MHz, DMSO-d₆) δ 7.94 (d, J=1.6 Hz, 1H), 7.80-7.70 (m, 2H), 7.68 (dd, J=8.0, 1.6 Hz, 1H), 7.45 (d, J=6.3 Hz, 2H), 7.28 (dt, J=6.0, 3.2 Hz, 2H), 7.19 (d, J=7.3 Hz, 1H), 6.97-6.89 (m, 1H), 5.42 (s, 2H), 5.01 (h, J=6.6 Hz, 1H), 3.67 (q, J=7.1 Hz, 2H), 3.53 (s, 2H), 1.51 (d, J=6.6 Hz, 6H), 0.77 (t, J=7.1 Hz, 3H); MS (ES+): 485.20 (M+1); (ES−): 483.20 (M−1).

### Step-2: Preparation of 2-(2-((5-(2-aminobenzo[d]oxazol-6-yl)-1-isopropyl-1H-indazol-3-yl)methoxy)phenyl)acetic acid (105c)

Compound 105c was prepared according to the procedure reported in step-5 of scheme-2 from ethyl 2-(2-((5-(2-aminobenzo[d]oxazol-6-yl)-1-isopropyl-1H-indazol-3-yl)methoxy)phenyl)acetate (105b) (140 mg, 0.289 mmol) in THE/MeOH (4 mL, 1:1) using a solution of LiOH·H₂O (72.7 mg, 1.734 mmol) in water (1 mL) and heating at 50° ° C. for 1 h. This gave after workup and purification using method-G, 2-(2-((5-(2-aminobenzo[d]oxazol-6-yl)-1-isopropyl-1H-indazol-3-yl)methoxy)phenyl)acetic acid (105c) (70 mg, 53.1% yield) HCl salt as a white solid; 1H NMR (300 MHz, DMSO-d₆) δ 9.09 (s, 2H, D₂O exchangeable), 8.04 (s, 1H), 7.89 (d, J=1.6 Hz, 1H), 7.85-7.69 (m, 2H), 7.61 (dd, J=8.2, 1.7 Hz, 1H), 7.39 (d, J=8.2 Hz, 1H), 7.33-7.24 (m, 2H), 7.24-7.15 (m, 1H), 6.97-6.86 (m, 1H), 5.45 (s, 2H), 5.03 (p, J=6.6 Hz, 1H), 3.53 (s, 2H), 1.51 (d, J=6.6 Hz, 6H); MS (ES+): 457.20 (M+1); MS (ES−): 455.10 (M−1).

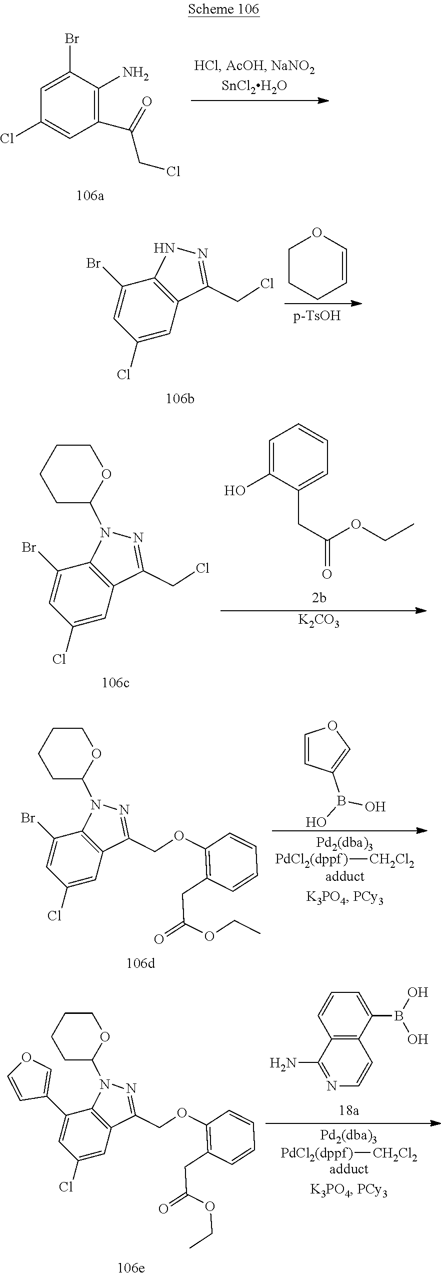

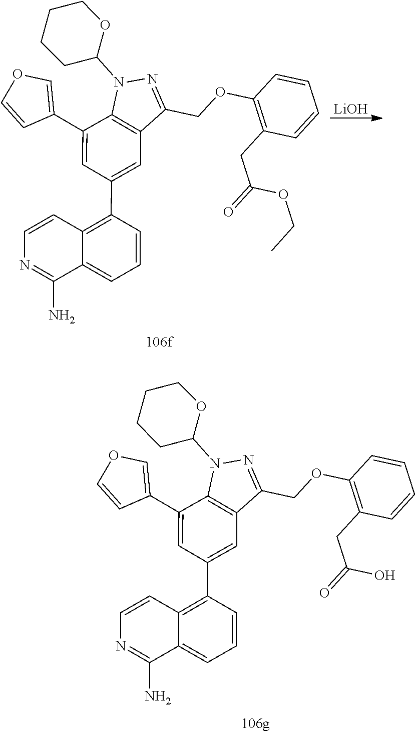

### Preparation of 2-(2-((5-(1-aminoisoquinolin-5-yl)-7-(furan-3-yl)-1-(tetrahydro-2H-pyran-2-yl)-1H-indazol-3-yl)methoxy)phenyl)acetic acid (106g)

### Step-1: Preparation of 7-bromo-5-chloro-3-(chloromethyl)-1H-indazole (106b)

To a stirred suspension of 1-(2-amino-3-bromo-5-chlorophenyl)-2-chloroethan-1-one (106a) (20 g, 70.68 mmol; CAS #1072925-14-4) in conc. HCl (300 mL) and acetic acid (300 mL) was added a solution of sodium nitrite (17.06 g, 247.39 mmol) in water (80 mL) at 0° C. and stirred for 20 minutes. To this, a solution of SnCl₂·2H₂O (79.74 g, 353.42 mmol) in conc. HCl (80 mL) was added and was stirred for 2 h at 0° C. To this mixture was added ice-water (1000 mL) and stirred for 10 minutes. Precipitate was filtered, washed with water, dried and filtered to afford 7-bromo-5-chloro-3-(chloromethyl)-1H-indazole (106b) (12.24 g, 62% yield) as an off white solid; 1H NMR (300 MHz, DMSO-d₆) δ 13.83 (s, 1H), 8.02 (d, J=1.7 Hz, 1H), 7.76 (d, J=1.7 Hz, 1H), 5.13 (s, 2H).

### Step-2: Preparation of 7-bromo-5-chloro-3-(chloromethyl)-1-(tetrahydro-2H-pyran-2-yl)-1H-indazole (106c)

Compound 106c was prepared according to the procedure reported in step-1 of scheme-9 from 7-bromo-5-chloro-3-(chloromethyl)-1H-indazole (106b) (5 g, 17.86 mmol) in DCM (40 mL) and THF (40 mL) using p-toluenesulphonic acid (0.461 g, 2.68 mmol) and 3,4-dihydro-2H-pyran (2.444 mL, 26.8 mmol) to afford after workup and purification using method-AJ, 7-bromo-5-chloro-3-(chloromethyl)-1-(tetrahydro-2H-pyran-2-yl)-1H-indazole (106c) (3.85 g, 59.2% yield) as a white solid; 1H NMR (300 MHz, DMSO-d₆) δ 8.07 (d, J=1.8 Hz, 1H), 7.84 (d, J=1.8 Hz, 1H), 6.33 (dd, J=10.0, 2.2 Hz, 1H), 5.14 (s, 2H), 3.95-3.84 (m, 1H), 3.80-3.66 (m, 1H), 2.47-2.35 (m, 1H), 2.13-1.97 (m, 2H), 1.83-1.61 (m, 1H), 1.61-1.48 (m, 2H); MS (ES+): 384.90 & 386.90 (M+Na).

### Step-3: Preparation of ethyl 2-(2-((7-bromo-5-chloro-1-(tetrahydro-2H-pyran-2-yl)-1H-indazol-3-yl)methoxy)phenyl)acetate (106d)

To a solution of 7-bromo-5-chloro-3-(chloromethyl)-1-(tetrahydro-2H-pyran-2-yl)-1H-indazole (106c) (3.85 g, 10.58 mmol) in DMF (50 mL) was added K₂CO₃ (4.38 g, 31.7 mmol) and ethyl 2-(2-hydroxyphenyl)acetate (2b) (3.81 g, 21.15 mmol). The mixture was degassed and filled with Ar, heated at 70° C. for 15 h. The mixture was poured into EtOAc and washed with water and brine. The organic layer was dried and concentrated, and the residue was purified using method-A to give ethyl 2-(2-((7-bromo-5-chloro-1-(tetrahydro-2H-pyran-2-yl)-1H-indazol-3-yl)methoxy)phenyl)acetate (106d) (2.1 g, 39.1% yield) as a yellow solid; 1H NMR (300 MHz, DMSO-d₆) δ 7.89 (d, J=1.8 Hz, 1H), 7.83 (d, J=1.8 Hz, 1H), 7.34-7.15 (m, 3H), 6.98-6.89 (m, 1H), 6.39-6.30 (m, 1H), 5.39 (s, 2H), 3.95-3.82 (m, 3H), 3.82-3.66 (m, 1H), 3.53 (s, 2H), 2.47-2.38 (m, 1H), 2.13-1.97 (m, 2H), 1.83-1.63 (m, 1H), 1.63-1.43 (m, 2H), 0.95 (t, J=7.1 Hz, 3H); MS (ES+): 529.10 & 531.10 (M+Na).

### Step-4: Preparation of ethyl 2-(2-((5-chloro-7-(furan-3-yl)-1-(tetrahydro-2H-pyran-2-yl)-1H-indazol-3-yl)methoxy)phenyl)acetate (106e)

Compound 106e was prepared according to the procedure reported in step-5 of scheme-1, from ethyl 2-(2-((7-bromo-5-chloro-1-(tetrahydro-2H-pyran-2-yl)-1H-indazol-3-yl)methoxy)phenyl)acetate (106d) (300 mg, 0.591 mmol) in dioxane/THF (4 mL, 1:1) using furan-3-ylboronic acid (99 mg, 0.886 mmol), K₃PO₄ (2M aqueous solution, 1.182 mL, 2.363 mmol), PCy₃ (33.1 mg, 0.118 mmol), PdCl₂(dppf)-CH₂Cl₂ adduct (48.2 mg, 0.059 mmol) and Pd₂(dba)₃ (54.1 mg, 0.059 mmol) to afford after workup and purification using method-AP, ethyl 2-(2-((5-chloro-7-(furan-3-yl)-1-(tetrahydro-2H-pyran-2-yl)-1H-indazol-3-yl)methoxy)phenyl)acetate (106e) (270 mg, 92% yield) as a clear oil; MS (ES+): 517.10 (M+Na).

### Step-5: Preparation of ethyl 2-(2-((5-(1-aminoisoquinolin-5-yl)-7-(furan-3-yl)-1-(tetrahydro-2H-pyran-2-yl)-1H-indazol-3-yl)methoxy)phenyl)acetate (106f)

Compound 106f was prepared according to the procedure reported in step-5 of scheme-1, from ethyl 2-(2-((5-chloro-7-(furan-3-yl)-1-(tetrahydro-2H-pyran-2-yl)-1H-indazol-3-yl)methoxy)phenyl)acetate (106e) (270 mg, 0.545 mmol) in dioxane/THF (4 mL, 1:1) using (1-aminoisoquinolin-5-yl)boronic acid (18a) (154 mg, 0.818 mmol), K₃PO₄ (2M aqueous solution, 1.091 mL, 2.182 mmol), PCy₃ (30.6 mg, 0.109 mmol), PdCl₂(dppf)-CH₂Cl₂ adduct (44.5 mg, 0.055 mmol) and Pd₂(dba)₃ (50.0 mg, 0.055 mmol) to afford after workup and purification using method-AM, ethyl 2-(2-((5-(1-aminoisoquinolin-5-yl)-7-(furan-3-yl)-1-(tetrahydro-2H-pyran-2-yl)-1H-indazol-3-yl)methoxy)phenyl)acetate (106f) (140 mg, 42.6% yield) as a clear oil; MS (ES+): 603.30 (M+1).

### Step-5: Preparation of 2-(2-((5-(1-aminoisoquinolin-5-yl)-7-(furan-3-yl)-1-(tetrahydro-2H-pyran-2-yl)-1H-indazol-3-yl)methoxy)phenyl)acetic acid (106g)

Compound 106g was prepared according to the procedure reported in step-2 of scheme-1 from ethyl 2-(2-((5-(1-aminoisoquinolin-5-yl)-7-(furan-3-yl)-1-(tetrahydro-2H-pyran-2-yl)-1H-indazol-3-yl)methoxy)phenyl)acetate (106f) (140 mg, 0.232 mmol) in MeOH/THF (6 mL, 1:1) using a solution of LiOH·H₂O (58.5 mg, 1.394 mmol) in water (2 mL) to afford after workup and purification using method-G, 2-(2-((5-(1-aminoisoquinolin-5-yl)-7-(furan-3-yl)-1-(tetrahydro-2H-pyran-2-yl)-1H-indazol-3-yl)methoxy)phenyl)acetic acid (106g) (45 mg, 33.7% yield) HCl salt as a white solid; H NMR (300 MHz, DMSO-d₆) δ 13.41 (s, 1H, D₂O exchangeable), 12.05 (s, 1H, D₂O exchangeable), 9.22 (s, 2H, D₂O exchangeable), 8.64 (d, J=8.4 Hz, 1H), 8.08-7.97 (m, 2H), 7.97-7.89 (m, 2H), 7.89-7.80 (m, 1H), 7.63 (d, J=7.2 Hz, 1H), 7.32 (d, J=1.6 Hz, 1H), 7.26 (d, J=4.2 Hz, 2H), 7.19 (d, J=7.3 Hz, 1H), 7.04 (d, J=7.2 Hz, 1H), 6.96-6.84 (m, 2H), 5.53-5.48 (m, 1H), 5.48-5.44 (m, 2H), 3.89-3.77 (m, 1H), 3.49 (s, 2H), 3.15-3.01 (m, 1H), 2.48-2.38 (m, 1H), 2.03-1.86 (m, 2H), 1.57-1.32 (m, 3H); MS (ES+): 575.30 (M+1); (ES−): 573.20 (M−1).

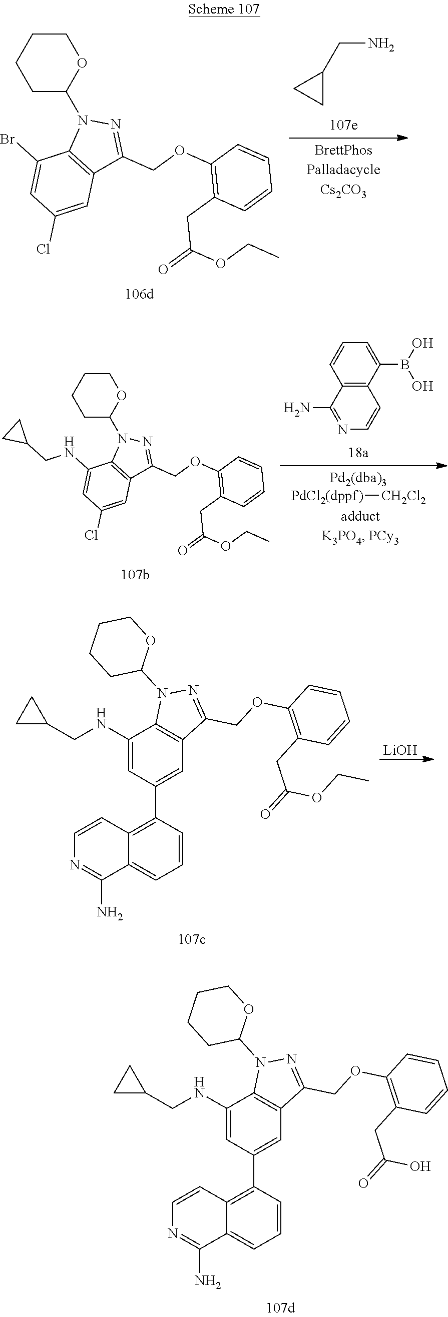

### Preparation of 2-(2-((5-(1-aminoisoquinolin-5-yl)-7-((cyclopropylmethyl)amino)-1-(tetrahydro-2H-pyran-2-yl)-1H-indazol-3-yl)methoxy)phenyl)acetic acid (107d)

### Step-1: Preparation of ethyl 2-(2-((5-chloro-7-(cyclopropylmethylamino)-1-(tetrahydro-2H-pyran-2-yl)-1H-indazol-3-yl)methoxy)phenyl)acetate (107b)

To a degassed solution of ethyl 2-(2-((7-bromo-5-chloro-1-(tetrahydro-2H-pyran-2-yl)-1H-indazol-3-yl)methoxy)phenyl)acetate (106d) (300 mg, 0.591 mmol) in MeCN (5 mL) was added cyclopropylmethanamine (107a) (0.101 mL, 1.182 mmol), BrettPhos Palladacycle (23.57 mg, 0.030 mmol) and Cs₂CO₃ (577 mg, 1.772 mmol). The mixture was degassed and filled with Ar and heated in a microwave at 90° C. for 1 h. The mixture was diluted with EtOAc and washed with water and brine. The organic layer was dried, filtered and concentrated, and the residue was purified using method-AQ to give ethyl 2-(2-((5-chloro-7-(cyclopropylmethylamino)-1-(tetrahydro-2H-pyran-2-yl)-1H-indazol-3-yl)methoxy)phenyl)acetate (107b) (250 mg, 85% yield) as a clear oil; MS (ES+): 521.20 (M+Na).

### Step-2: Preparation of ethyl 2-(2-((5-(1-aminoisoquinolin-5-yl)-7-(cyclopropylmethylamino)-1-(tetrahydro-2H-pyran-2-yl)-1H-indazol-3-yl)methoxy)phenyl)acetate (107c)

Compound 107c was prepared according to the procedure reported in step-5 of scheme-1, from ethyl 2-(2-((5-chloro-7-(cyclopropylmethylamino)-1-(tetrahydro-2H-pyran-2-yl)-1H-indazol-3-yl)methoxy)phenyl)acetate (107b) (250 mg, 0.502 mmol) in dioxane/THF (4 mL, 1:1) using (1-aminoisoquinolin-5-yl)boronic acid (18a) (142 mg, 0.753 mmol), K₃PO₄ (2M aqueous solution, 1.004 mL, 2.008 mmol), PCy₃ (28.2 mg, 0.100 mmol), PdCl₂(dppf)-CH₂Cl₂ adduct (41.0 mg, 0.050 mmol) and Pd₂(dba)₃ (46.0 mg, 0.050 mmol) to afford after workup and purification using method-AM, ethyl 2-(2-((5-(1-aminoisoquinolin-5-yl)-7-(cyclopropylmethylamino)-1-trahydro-2H-pyran-2-yl)-1H-indazol-3-yl)methoxy)phenyl)acetate (107c) (30 mg, 9.87% yield) as a clear oil; MS (ES+): 606.30 (M+1);

### Step-3: Preparation of 2-(2-((5-(1-aminoisoquinolin-5-yl)-7-((cyclopropylmethyl)amino)-1-(tetrahydro-2H-pyran-2-yl)-1H-indazol-3-yl)methoxy)phenyl)acetic acid (107d)

Compound 107d was prepared according to the procedure reported in step-2 of scheme-1 from ethyl 2-(2-((5-(1-aminoisoquinolin-5-yl)-7-(cyclopropylmethylamino)-1-(tetrahydro-2H-pyran-2-yl)-1H-indazol-3-yl)methoxy)phenyl)acetate (107c) (30 mg, 0.050 mmol) in MeOH/THF (6 mL, 1:1) using a solution of LiOH·H₂O (12.47 mg, 0.297 mmol) in water (2 mL) to afford after workup and purification using method-G, 2-(2-((5-(1-aminoisoquinolin-5-yl)-7-((cyclopropylmethyl)amino)-1-(tetrahydro-2H-pyran-2-yl)-1H-indazol-3-yl)methoxy)phenyl)acetic acid (107d) (5 mg, 17.48% yield) HCl salt as a white solid; ¹H NMR (300 MHz, DMSO-d₆) δ 12.99 (s, 1H, D₂O exchangeable), 9.06 (s, 2H, D₂O) exchangeable), 8.56 (d, J=8.4 Hz, 1H), 7.98-7.91 (m, 1H), 7.83 (t, J=7.8 Hz, 1H), 7.58 (d, J=7.4 Hz, 1H), 7.24-7.20 (m, 2H), 7.18-7.14 (m, 1H), 7.12-7.06 (m, 2H), 6.94-6.85 (m, 1H), 6.47-6.42 (m, 1H), 6.20 (m, 1H, D₂O exchangeable), 6.05-5.92 (m, 1H), 5.36 (s, 2H), 4.20-4.10 (m, 1H), 3.97-3.81 (m, 1H), 3.47-3.46 (m, 2H), 3.03-2.91 (m, 2H), 2.38-2.24 (m, 1H), 2.11-1.93 (m, 2H), 1.81-1.64 (m, 3H), 1.29-1.19 (m, 1H), 0.64-0.54 (m, 2H), 0.34-0.24 (m, 2H); MS (ES+): 578.20 (M+1); MS (ES−): 576.10 (M−1).

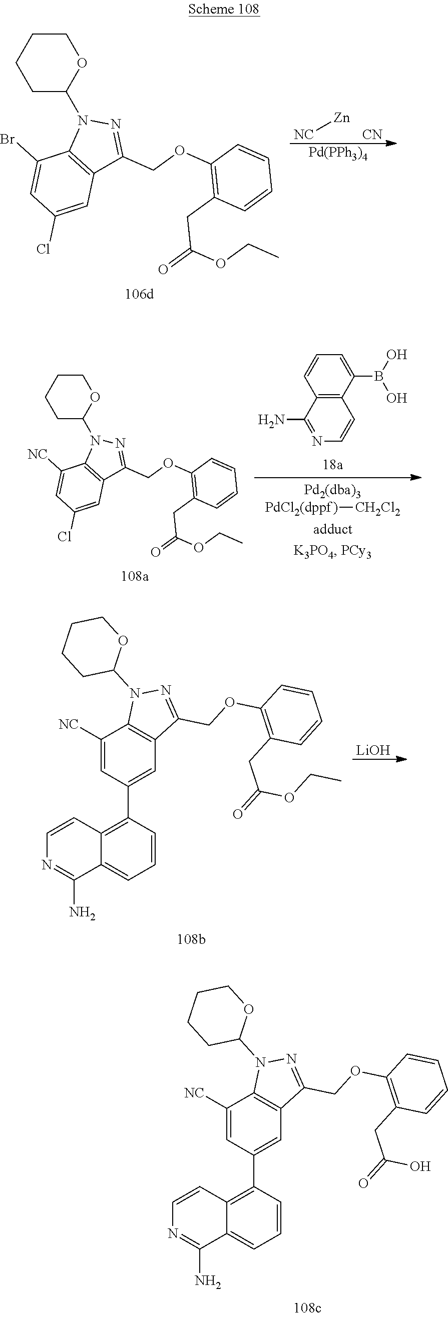

### Preparation of 2-(2-((5-(1-aminoisoquinolin-5-yl)-7-cyano-1-(tetrahydro-2H-pyran-2-yl)-1H-indazol-3-yl)methoxy)phenyl)acetic acid (108c)

### Step-1: Preparation of ethyl 2-(2-((5-chloro-7-cyano-1-(tetrahydro-2H-pyran-2-yl)-1H-indazol-3-yl)methoxy)phenyl)acetate (108a)

A solid mixture of ethyl 2-(2-((7-bromo-5-chloro-1-(tetrahydro-2H-pyran-2-yl)-1H-indazol-3-yl)methoxy)phenyl)acetate (106d) (300 mg, 0.591 mmol), dicyanozine (104 mg, 0.886 mmol), Pd(PPh₃)+(102 mg, 0.089 mmol) was purged with nitrogen for 10 min, and added anhydrous MeCN (5 mL). The mixture was degassed filled with Ar and heated on a microwave reactor at 90° ° C. for 1 h. The reaction mixture was cooled to room temperature, diluted with ethyl acetate (100 mL), and brine (100 mL) and extracted with ethyl acetate (2×50 mL). The combined organics were washed with brine, dried, filtered, evaporated to dryness. The residue obtained was purified using method-J to afford ethyl 2-(2-((5-chloro-7-cyano-1-(tetrahydro-2H-pyran-2-yl)-1H-indazol-3-yl)methoxy)phenyl)acetate (108a) (200 mg, 74.6% yield) as a white solid; ¹H NMR (300 MHz, DMSO-d₆) δ 8.32-8.18 (m, 2H), 7.33-7.15 (m, 3H), 7.03-6.81 (m, 1H), 6.07 (dd, J=9.7, 2.2 Hz, 1H), 5.43 (s, 2H), 3.95-3.67 (m, 4H), 3.54 (s, 2H), 2.46-2.29 (m, 1H), 2.23-1.96 (m, 2H), 1.86-1.43 (m, 3H), 0.95 (t, J=7.1 Hz, 3H); MS (ES+): 476.10 & 478.10 (M+Na).

### Step-2: Preparation of ethyl 2-(2-((5-(1-aminoisoquinolin-5-yl)-7-cyano-1-(tetrahydro-2H-pyran-2-yl)-1H-indazol-3-yl)methoxy)phenyl)acetate (108b)

Compound 108b was prepared according to the procedure reported in step-5 of scheme-1, from ethyl 2-(2-((5-chloro-7-cyano-1-(tetrahydro-2H-pyran-2-yl)-1H-indazol-3-yl)methoxy)phenyl)acetate (108a) (200 mg, 0.441 mmol) in dioxane/THF (4 mL, 1:1) using (1-aminoisoquinolin-5-yl)boronic acid (18a), K₃PO₄ (2M aqueous solution, 0.881 mL, 1.762 mmol), PCy₃ (24.71 mg, 0.088 mmol), PdCl₂(dppf)-CH₂Cl₂ adduct (36.0 mg, 0.044 mmol) and Pd₂(dba)₃ (40.3 mg, 0.044 mmol) to afford after workup and purification using method-AM, ethyl 2-(2-((5-(1-aminoisoquinolin-5-yl)-7-cyano-1-(tetrahydro-2H-pyran-2-yl)-1H-indazol-3-yl)methoxy)phenyl)acetate (108b) (90 mg, 36.4% yield) as a clear oil; MS (ES+): 562.30 (M+1).

### Step-3: Preparation of 2-(2-((5-(1-aminoisoquinolin-5-yl)-7-cyano-1-(tetrahydro-2H-pyran-2-yl)-1H-indazol-3-yl)methoxy)phenyl)acetic acid (108c)

Compound 108c was prepared according to the procedure reported in step-2 of scheme-1 from ethyl 2-(2-((5-(1-aminoisoquinolin-5-yl)-7-cyano-1-(tetrahydro-2H-pyran-2-yl)-1H-indazol-3-yl)methoxy)phenyl)acetate (108b) (90 mg, 0.160 mmol) in MeOH/THF (6 mL, 1:1) using a solution of LiOH·H₂O (40.3 mg, 0.961 mmol) in water (2 mL) to afford after workup and purification using method-G, 2-(2-((5-(1-aminoisoquinolin-5-yl)-7-cyano-1-(tetrahydro-2H-pyran-2-yl)-1H-indazol-3-yl)methoxy)phenyl)acetic acid (108c) (30 mg, 35.1% yield) HCl salt as a white solid; ¹H NMR (300 MHz, DMSO-d₆) δ 13.36 (s, 1H, D₂O exchangeable), 11.99 (s, 1H, D₂O exchangeable), 9.20 (s, 2H, D₂O exchangeable), 8.67 (d, J=8.4 Hz, 1H), 8.31 (d, J=1.6 Hz, 1H), 8.15 (d, J=1.5 Hz, 1H), 8.05-7.97 (m, 1H), 7.87 (t, 1H), 7.64 (d, J=7.2 Hz, 1H), 7.29-7.21 (m, 2H), 7.21-7.12 (m, 1H), 6.98-6.85 (m, 2H), 6.21-6.10 (m, 1H), 5.49 (s, 2H), 4.01-3.88 (m, 1H), 3.88-3.74 (m, 1H), 3.46 (s, 2H), 2.49-2.38 (m, 1H), 2.26-2.03 (m, 2H), 1.89-1.67 (m, 1H), 1.67-1.51 (m, 2H); MS (ES+): 534.20 (M+1); MS (ES−): 532.20 (M−1).

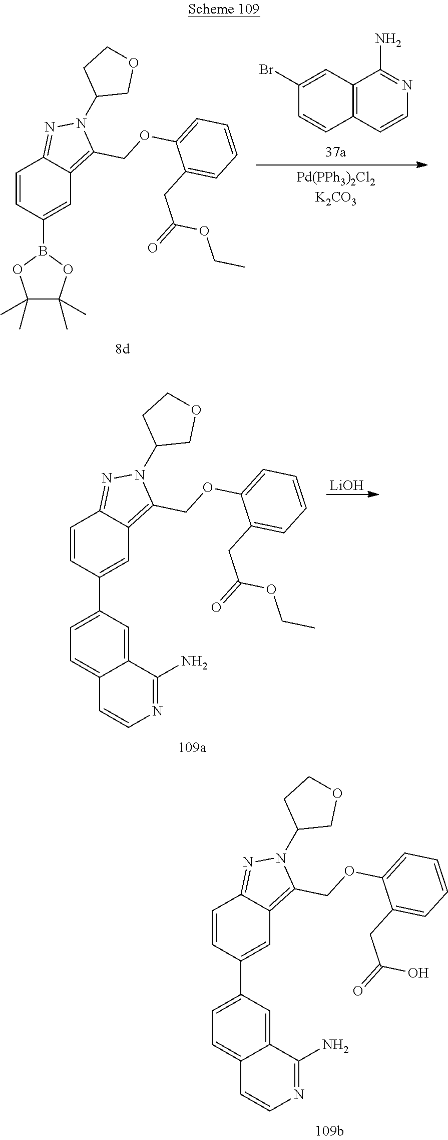

### Preparation of 2-(2-((5-(1-aminoisoquinolin-7-yl)-2-(tetrahydrofuran-3-yl)-2H-indazol-3-yl)methoxy)phenyl)acetic acid (109b)

### Step-1, Preparation of ethyl 2-(2-((5-(1-aminoisoquinolin-7-yl)-2-(tetrahydrofuran-3-yl)-2H-indazol-3-yl)methoxy)phenyl)acetate (109a)

Compound 109a was prepared according to the procedure reported in step-5 of scheme-8 from ethyl 2-(2-((2-(tetrahydrofuran-3-yl)-5-(4,4,5,5-tetramethyl-1,3,2-dioxaborolan-2-yl)-2H-indazol-3-yl)methoxy)phenyl)acetate (8d) in Dioxanes (5 mL) using 7-bromoisoquinolin-1-amine (37a), Pd(PPh₃)₂Cl₂ and a solution of K₂CO₃ in Water (0.5 mL) to afford after workup and purification using method-C, ethyl 2-(2-((5-(1-aminoisoquinolin-7-yl)-2-(tetrahydrofuran-3-yl)-2H-indazol-3-yl)methoxy)phenyl)acetate (109a) (96 mg, 0.184 mmol) as a yellow oil; MS (ES+): 523.2 (M+1), (ES−): 521.2 (M−1).

### Step-2, Preparation of 2-(2-((5-(1-aminoisoquinolin-7-yl)-2-(tetrahydrofuran-3-yl)-2H-indazol-3-yl)methoxy)phenyl)acetic acid (109b)

Compound 109b was prepared according to the procedure reported in step-2 of scheme-1 from ethyl 2-(2-((5-(1-aminoisoquinolin-7-yl)-2-(tetrahydrofuran-3-yl)-2H-indazol-3-yl)methoxy)phenyl)acetate (109a) (96 mg, 0.184 mmol) in THF (3 mL) using a solution of LiOH·H₂O) (3.8 mgs, 0.552 mmol) in water (1 mL) to afford after workup and purification using method-M, 2-(2-((5-(1-aminoisoquinolin-7-yl)-2-(tetrahydrofuran-3-yl)-2H-indazol-3-yl)methoxy)phenyl)acetic acid (109b) (44 mg, 0.089 mmol) HCl salt as a white solid; ¹H NMR (300 MHz, DMSO-d₆) δ 13.52 (s, 1H, D₂O exchangeable), 9.32 (s, 2H, D₂O exchangeable), 9.04 (d, J=1.8 Hz, 1H), 8.52 (s, 1H), 8.44 (dd, J=8.5, 1.6 Hz, 1H), 8.03 (d, J=8.5 Hz, 1H), 7.92 (dd, J=9.2, 1.7 Hz, 1H), 7.83 (d, J=9.0 Hz, 1H), 7.68 (d, J=7.0 Hz, 1H), 7.40-7.28 (m, 2H), 7.24 (dd, J=7.0, 4.9 Hz, 2H), 6.97 (td, J=7.0, 1.8 Hz, 1H), 5.79-5.64 (m, 2H), 5.61-5.43 (m, 1H), 4.24-4.12 (m, 2H), 4.02-3.90 (m, 2H), 3.50 (s, 2H), 2.49-2.36 (m, 2H); MS (ES+): 495.2 (M+1); (ES−): 493.1 (M−1).

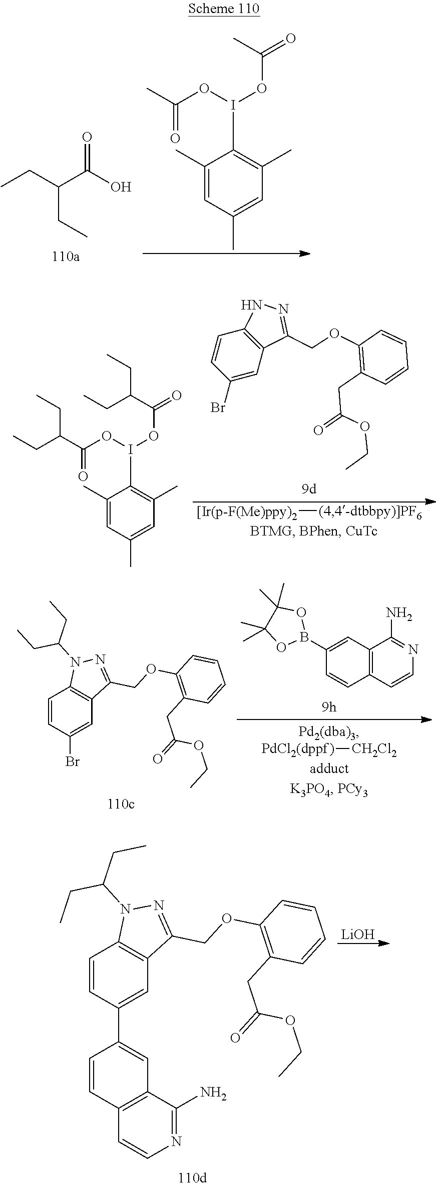

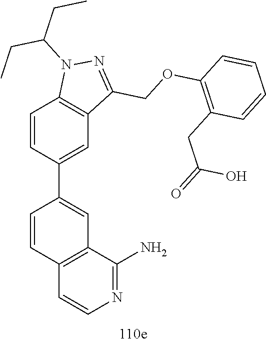 [SMILES: CCC(CC)n1nc(COc2ccccc2CC(=O)O)c2cc(-c3ccc4ccnc(N)c4c3)ccc21]

### Preparation of 2-(2-((5-(1-aminoisoquinolin-7-yl)-1-(pentan-3-yl)-1H-indazol-3-yl)methoxy)phenyl)acetic acid (110e)

### Step-1: Preparation of iodomesitylene bis(2-ethylbutanoate) (110b)

Compound 110b was prepared according to the procedure reported in step-5 of scheme-9 from Iodomesitylene diacetate (1 g, 2.75 mmol) in toluene (12 mL) using 2-ethylbutanoic acid (0.670 g, 5.77 mmol) (110a) to afford after workup iodomesitylene bis(2-ethylbutanoate) (110b) (950 mg, 72.6% yield), which was used the next step without further purification.

### Step-2: Preparation of ethyl 2-(2-((5-bromo-1-(pentan-3-yl)-1H-indazol-3-yl)methoxy)phenyl)acetate (110c)

Compound 110c was prepared according to the procedure reported in step-6 of scheme-9 from ethyl 2-(2-((5-bromo-1H-indazol-3-yl)methoxy)phenyl)acetate (9d) (388 mg, 0.997 mmol) in 1,4-dioxane (10 mL) using iodomesitylene bis(2-ethylbutanoate) (110b) (950 mg, 1.994 mmol), [Ir(p-F(Me)ppy)2-(4,4′-dtbbpy)]PF6 (9.75 mg, 9.97 μmol), 2-tert-butyl-1,1,3,3-tetramethylguanidine (BTMG, 342 mg, 1.994 mmol), 4,7-diphenyl-1,10-phenanthroline (Bphen, 99 mg, 0.299 mmol) and copper(I) thiophene-2-carboxylate (CuTC, 38.0 mg, 0.199 mmol) to afford after workup and purification using method-J, ethyl 2-(2-((5-bromo-1-(pentan-3-yl)-1H-indazol-3-yl)methoxy)phenyl)acetate (110c) (210 mg, 45.8% yield) as a clear oil; MS (ES+): 459.1 and 461.1 (M+1).

### Step-3: Preparation of ethyl 2-(2-((5-(1-aminoisoquinolin-7-yl)-1-(pentan-3-yl)-1H-indazol-3-yl)methoxy)phenyl)acetate (110d)

Compound 110d was prepared according to the procedure reported in step-5 of scheme-7 from ethyl 2-(2-((5-bromo-)-(pentan-3-yl)-1H-indazol-3-yl)methoxy)phenyl)acetate (110c) (210 mg, 0.457 mmol) in dioxane/2-MeTHF (4 mL, 3:1) using 7-(4,4,5,5-tetramethyl-1,3,2-dioxaborolan-2-yl)isoquinolin-1-amine (9h) (185 mg, 0.686 mmol), K₃PO₄ (2M aqueous solution, 0.914 mL, 1.829 mmol), PCy₃ (25.6 mg, 0.091 mmol), Pd₂(dba)₃ (41.9 mg, 0.046 mmol) and PdCl₂(dppf)-CH₂Cl₂ adduct (37.3 mg, 0.046 mmol) to afford after workup and purification using method-U, ethyl 2-(2-((5-(1-aminoisoquinolin-7-yl)-1-(pentan-3-yl)-1H-indazol-3-yl)methoxy)phenyl)acetate (110d) (78 mg, 32.6% yield) as a clear oil; MS (ES+): 523.3 (M+1); (ES−): 557.2 (M+CI).

### Step-4: Preparation of 2-(2-((5-(1-aminoisoquinolin-7-yl)-1-(pentan-3-yl)-1H-indazol-3-yl)methoxy)phenyl)acetic acid (110e)

Compound 110e was prepared according to the procedure reported step-2 of scheme-1 from ethyl 2-(2-((5-(1-aminoisoquinolin-7-yl)-1-(pentan-3-yl)-1H-indazol-3-yl)methoxy)phenyl)acetate (110d) (78 mg, 0.149 mmol) in THF (3 mL) using a solution of LiOH·H₂O (57.5 mg, 1.371 mmol) in water (1 mL) to afford after workup and purification using method-M, 2-(2-((5-(1-aminoisoquinolin-7-yl)-1-(pentan-3-yl)-1H-indazol-3-yl)methoxy)phenyl)acetic acid (110e) (28 mg, 12.38% yield) HCl salt as a white solid; 1H NMR (300 MHz, DMSO-d₆) δ 13.23 (s, 1H, D₂O) exchangeable), 12.14 (s, 1H, D₂O exchangeable), 9.13 (s, 2H, D₂O exchangeable), 8.94 (s, 1H), 8.41 (dd, J=8.5, 1.6 Hz, 1H), 8.29 (s, 1H), 8.05 (d, J=8.6 Hz, 1H), 7.98-7.90 (m, 2H), 7.70 (d, J=6.9 Hz, 1H), 7.32-7.22 (m, 3H), 7.19 (dd, J=7.7, 1.5 Hz, 1H), 6.90 (td, J=7.2, 1.5 Hz, 1H), 5.52 (s, 2H), 4.64-4.50 (m, 1H), 3.53 (s, 2H), 2.04-1.83 (m, 4H), 0.66 (t, J=7.2 Hz, 6H); MS (ES+), 495.3 (M+1); (ES−), 493.2 (M−1).

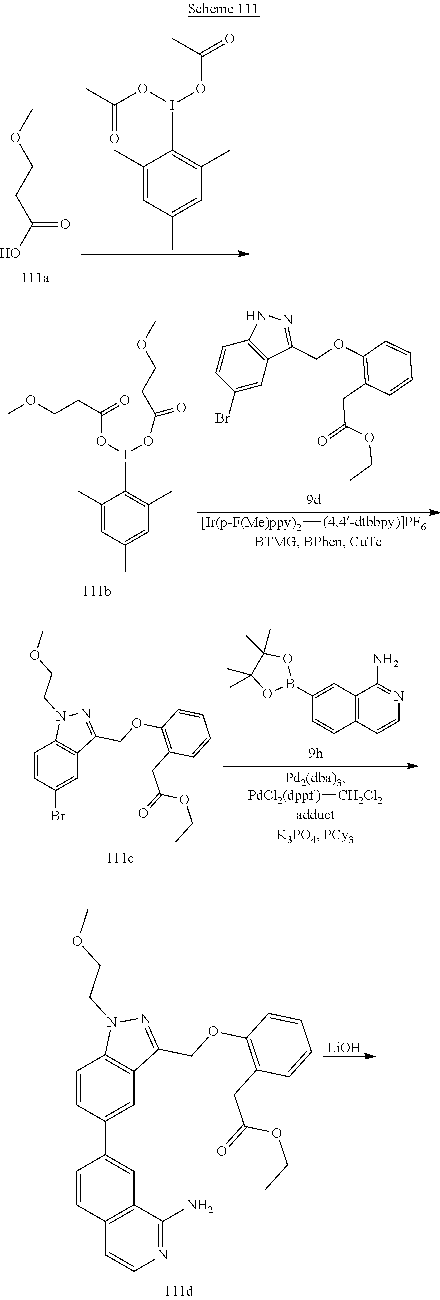

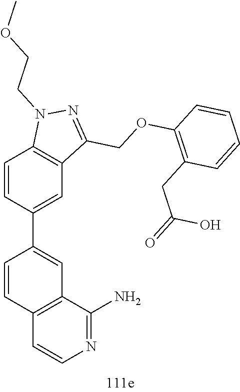 [SMILES: COCCn1nc(COc2ccccc2CC(=O)O)c2cc(-c3ccc4ccnc(N)c4c3)ccc21]

### Preparation of 2-(2-((5-(1-aminoisoquinolin-7-yl)-1-(2-methoxyethyl)-1H-indazol-3-yl)methoxy)phenyl)acetic acid (111e)

### Step-1: Preparation of iodomesitylene bis(3-methoxypropanoate) (111b)

Compound 111b was prepared according to the procedure reported in step-5 of scheme-9 from Iodomesitylene diacetate (500 mg, 1.373 mmol) in toluene (12 mL) using 3-methoxypropanoic acid (111a) (300 mg, 2.88 mmol) to afford after workup iodomesitylene bis(3-methoxypropanoate) (111b) (620 mg, 100% yield), which was used the next step without further purification.

### Step-2: Preparation of ethyl 2-(2-((5-bromo-1-(2-methoxyethyl)-1H-indazol-3-yl)methoxy)phenyl)acetate (111c)

Compound 111c was prepared according to the procedure reported in step-6 of scheme-9 from ethyl 2-(2-((5-bromo-1H-indazol-3-yl)methoxy)phenyl)acetate (9d) (262 mg, 0.674 mmol) in 1,4-dioxane (10 mL) using Iodomesitylene bis(3-methoxypropanoate) (111b) (610 mg, 1.349 mmol), [Ir(p-F(Me)ppy)2-(4,4′-dtbbpy)]PF₆ (6.60 mg, 6.74 μmol), 2-tert-butyl-1,1,3,3-tetramethylguanidine (BTMG, 231 mg, 1.349 mmol), 4,7-diphenyl-1,10-phenanthroline (Bphen, 67.2 mg, 0.202 mmol) and copper(I) thiophene-2-carboxylate (CuTC, 25.7 mg, 0.135 mmol) to afford after workup and purification using method-J, ethyl 2-(2-((5-bromo-1-(2-methoxyethyl)-1H-indazol-3-yl)methoxy)phenyl)acetate (111c) (207.5 mg, 068.8% yield) as a clear oil. MS (ES+): 447.1 and 449.1 (M+1).

### Step-3: Preparation of ethyl 2-(2-((5-(1-aminoisoquinolin-7-yl)-1-(2-methoxyethyl)-1H-indazol-3-yl)methoxy)phenyl)acetate (111d)

Compound 111d was prepared according to the procedure reported in step-5 of scheme-7 from ethyl 2-(2-((5-bromo-1-(2-methoxyethyl)-1H-indazol-3-yl)methoxy)phenyl)acetate (111c) (207 mg, 0.463 mmol) in dioxane/2-MeTHF (4 mL, 3:1) using 7-(4,4,5,5-tetramethyl-1,3,2-dioxaborolan-2-yl)isoquinolin-1-amine (9h) (188 mg, 0.694 mmol), K₃PO₄ (2M aqueous solution, 0.926 mL, 1.851 mmol), PCy₃ (26.0 mg, 0.093 mmol), PdCl₂(dppf)-CH₂Cl₂ adduct (37.8 mg, 0.046 mmol) and Pd₂(dba)₃ (42.4 mg, 0.046 mmol) to afford after workup and purification using method-U, ethyl 2-(2-((5-(1-aminoisoquinolin-7-yl)-1-(2-methoxyethyl)-1H-indazol-3-yl)methoxy)phenyl)acetate (111d) (98 mg, 41.5% yield) as a clear oil; MS (ES+): 511.3 (M+1).

### Step-4: Preparation of 2-(2-((5-(1-aminoisoquinolin-7-yl)-1-(2-methoxyethyl)-1H-indazol-3-yl)methoxy)phenyl)acetic acid (111e)

Compound 111e was prepared according to the procedure reported step-2 of scheme-1 from ethyl 2-(2-((5-(1-aminoisoquinolin-7-yl)-1-(2-methoxyethyl)-1H-indazol-3-yl)methoxy)phenyl)acetate (111d) (98 mg, 0.192 mmol, 41.5% yield) in THF (3 mL) using a solution of LiOH·H₂O (58.3 mg, 1.388 mmol) in water (1 mL) to afford after workup and purification using method-M, 2-(2-((5-(1-aminoisoquinolin-7-yl)-1-(2-methoxyethyl)-1H-indazol-3-yl)methoxy)phenyl)acetic acid (111e) (42 mg, 18.81% yield) HCl salt as an off white solid; ¹H NMR (300 MHz, DMSO-d₆) δ 13.26 (s, 1H, D₂O exchangeable), 12.12 (s, 1H, D₂O exchangeable), 9.14 (s, 2H, D₂O exchangeable), 8.96 (s, 1H), 8.43 (dd, J=8.6, 1.6 Hz, 1H), 8.30 (s, 1H), 8.05 (d, J=8.5 Hz, 1H), 8.00 (dd, J=9.0, 1.7 Hz, 1H), 7.89 (d, J=8.9 Hz, 1H), 7.69 (d, J=6.9 Hz, 1H), 7.33-7.25 (m, 3H), 7.24-7.18 (m, 1H), 7.00-6.88 (m, 1H), 5.49 (s, 2H), 4.65 (t, J=5.2 Hz, 2H), 3.81 (t, J=5.2 Hz, 2H), 3.54 (s, 2H), 3.21 (s, 3H); MS (ES+) 483.2 (M+1); (ES−) 481.3 (M−1); LC RT 1.894 min, 98.18%; Analysis calculated for C₂₈H₂₆N₄O₄·HCl·2H₂O: C, 60.59; H, 5.63; Cl, 6.39; N, 10.09; Found: C, 60.54; H, 5.48; Cl, 6.55; N, 10.23.

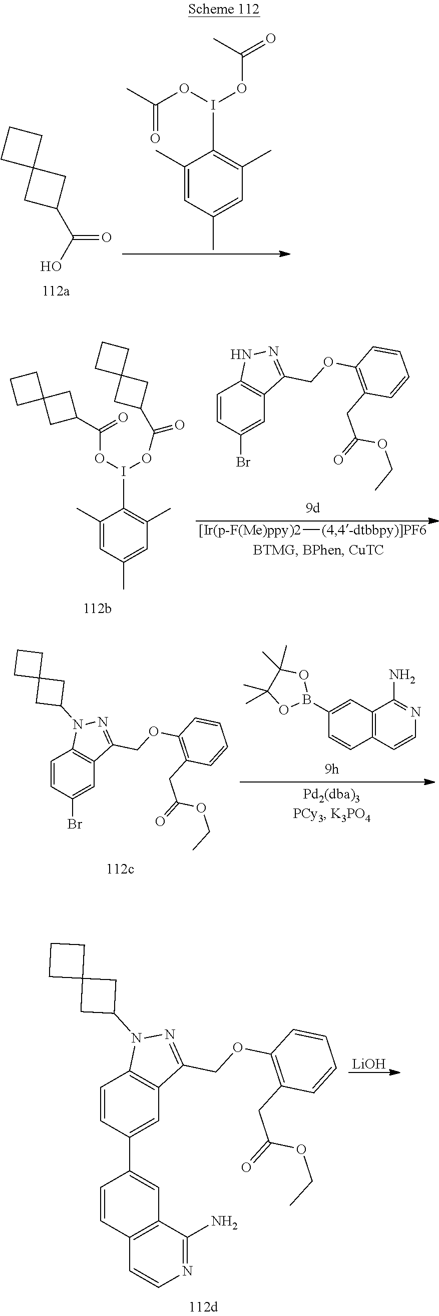

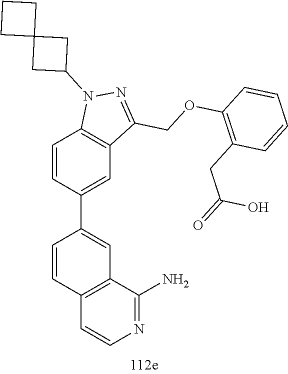 [SMILES: Nc1nccc2ccc(-c3ccc4c(c3)c(COc3ccccc3CC(=O)O)nn4C3CC4(CCC4)C3)cc12]

### Preparation of 2-(2-((5-(1-aminoisoquinolin-7-yl)-1-(spiro[3.3]heptan-2-yl)-1H-indazol-3-yl)methoxy)phenyl)acetic acid (112e)

### Step-1: Preparation of iodomesitylene bis(spiro[3.3]heptane-2-carboxylate) (112b)

Compound 112b was prepared according to the procedure reported in step-5 of scheme-9 from Iodomesitylene diacetate (500 mg, 1.373 mmol) in toluene (12 mL) using spiro[3.3]heptane-2-carboxylic acid (112a) (385 mg, 2.75 mmol) to afford after workup iodomesitylene bis(spiro[3.3]heptane-2-carboxylate) (112b) (705 mg, 1.344 mmol, 98% yield), which was used the next step without further purification.

### Step-2: Preparation of ethyl 2-(2-((5-bromo-1-(spiro[3,3]heptan-2-yl)-1H-indazol-3-yl)methoxy)phenyl)acetate (112c)

Compound 112c was prepared according to the procedure reported in step-6 of scheme-9 from ethyl 2-(2-((5-bromo-1H-indazol-3-yl)methoxy)phenyl)acetate (9d) (262 mg, 0.672 mmol) in 1,4-dioxane (10 mL) using iodomesitylene bis(spiro[3.3]heptane-2-carboxylate) (112b) (705 mg, 1.344 mmol), [Ir(p-F(Me)ppy)2-(4,4′-dtbbpy)]PF₆ (6.57 mg, 6.72 μmol), 2-tert-butyl-1,1,3,3-tetramethylguanidine (BTMG, 230 mg, 1.344 mmol), 4,7-diphenyl-1,10-phenanthroline (Bphen, 67.0 mg, 0.202 mmol) and copper(I) thiophene-2-carboxylate (CuTC, 25.6 mg, 0.134 mmol) to afford after workup and purification using method-J, ethyl 2-(2-((5-bromo-1-(spiro[3.3]heptan-2-yl)-1H-indazol-3-yl)methoxy)phenyl)acetate (112c) (125 mg, 38.5% yield) as a clear oil; MS (ES+): 483.1 and 485.1 (M+1).

### Step-3: Preparation of ethyl 2-(2-((5-(1-aminoisoquinolin-7-yl)-1-(spiro[3.3]heptan-2-yl)-1H-indazol-3-yl)methoxy)phenyl)acetate (112d)

To a degassed solution of ethyl 2-(2-((5-bromo-1-(spiro[3.3]heptan-2-yl)-1H-indazol-3-yl)methoxy)phenyl)acetate (112c) (122 mg, 0.252 mmol) in dioxane/2-MeTHF (4 mL, 3:1) was added 7-(4,4,5,5-tetramethyl-1,3,2-dioxaborolan-2-yl)isoquinolin-1-amine (9h) (102 mg, 0.379 mmol), K₃PO₄ (2M aqueous solution, 0.505 mL, 1.010 mmol), PCy₃ (14.15 mg, 0.050 mmol) and Pd₂(dba)₃ (23.11 mg, 0.025 mmol). The mixture was degassed and filled with Ar, then heated 100° C. for 5 h on oil bath. The mixture was diluted with EtOAc and washed with water and brine. The organic layer was dried and concentrated, the residue was purified using method-U to give ethyl 2-(2-((5-(1-aminoisoquinolin-7-yl)-1-(spiro[3.3]heptan-2-yl)-1H-indazol-3-yl)methoxy)phenyl)acetate (112d) (76 mg, 55.1% yield) as a clear oil; MS (ES+): 547.3 (M+1).

### Step-4: Preparation of 2-(2-((5-(1-aminoisoquinolin-7-yl)-1-(spiro[3.3]heptan-2-yl)-1H-indazol-3-yl)methoxy)phenyl)acetic acid (112e)

Compound 112e was prepared according to the procedure reported step-2 of scheme-1 from ethyl 2-(2-((5-(1-aminoisoquinolin-7-yl)-1-(spiro[3.3]heptan-2-yl)-1H-indazol-3-yl)methoxy)phenyl)acetate (112d) (76 mg, 0.139 mmol) in THF (3 mL) using a solution of LiOH·H₂O (31.8 mg, 0.757 mmol) in water (1 mL) to afford after workup and purification using method-M, 2-(2-((5-(1-aminoisoquinolin-7-yl)-1-(spiro[3.3]heptan-2-yl)-1H-indazol-3-yl)methoxy)phenyl)acetic acid (112e) (22 mg, 16.81% yield) HCl salt as a white solid; 1H NMR (300 MHz, DMSO-d₆) δ 13.10 (s, 1H, D₂O exchangeable), 12.13 (s, 1H, D₂O) exchangeable), 9.22-8.95 (m, 2H, D₂O exchangeable), 8.92 (s, 1H), 8.40 (dd, J=8.5, 1.6 Hz, 1H), 8.29 (s, 1H), 8.07-7.95 (m, 2H), 7.90 (d, J=8.9 Hz, 1H), 7.68 (d, J=6.9 Hz, 1H), 7.36-7.23 (m, 3H), 7.23-7.17 (m, 1H), 6.98-6.88 (m, 1H), 5.49 (s, 2H), 5.31-5.17 (m, 1H), 3.53 (s, 2H), 2.62 (d, J=8.1 Hz, 4H), 2.21 (t, J=7.3 Hz, 2H), 2.13-2.00 (m, 2H), 1.97-1.84 (m, 2H); MS (ES+): 519.2 (M+1); (ES−): 517.2 (M−1).

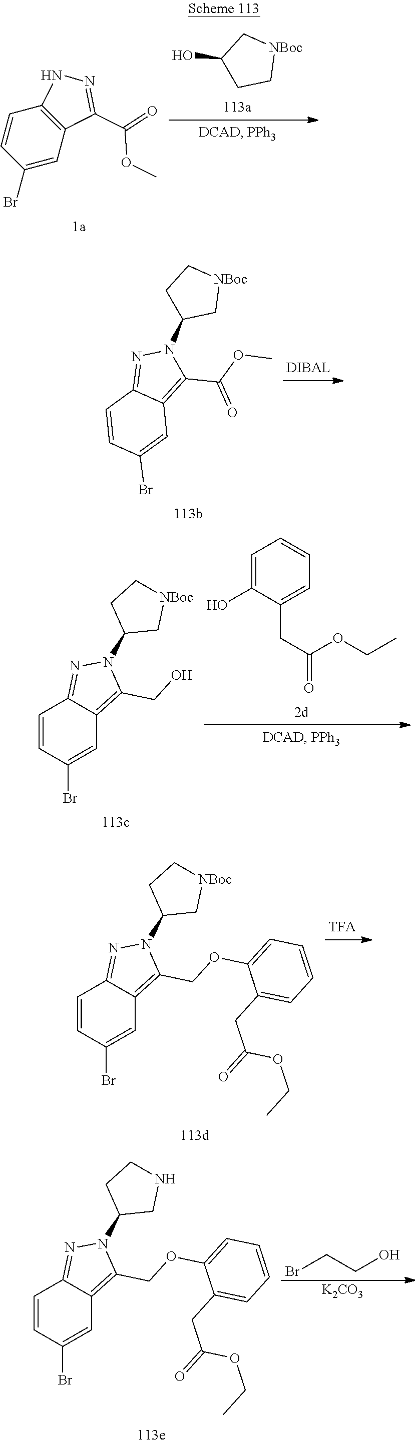

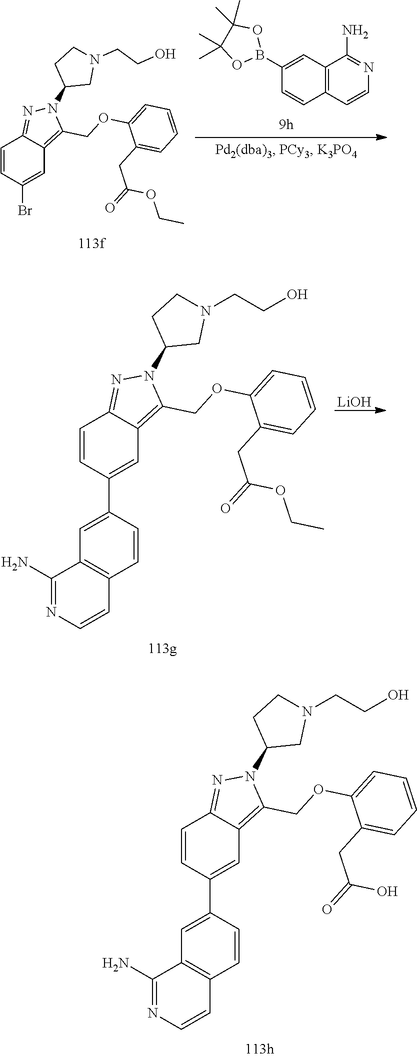

### Preparation of (S)-2-(2-((5-(1-aminoisoquinolin-7-yl)-2-(1-(2-hydroxyethyl)pyrrolidin-3-yl)-2H-indazol-3-yl)methoxy)phenyl)acetic acid (113h)

### Step-1: Preparation of (S)-methyl 5-bromo-2-(1-(tert-butoxycarbonyl)pyrrolidin-3-yl)-2H-indazole-3-carboxylate (113b)

Compound 113b was prepared according to the procedure reported in step-2 of scheme-2, from methyl 5-bromo-1H-indazole-3-carboxylate (1a) (5 g, 19.60 mmol) in THF using triphenylphosphine (10.28 g, 39.2 mmol), (R)-tert-butyl 3-hydroxypyrrolidine-1-carboxylate (113a) (7.34 g, 39.2 mmol; CAS #109431-87-0) and DCAD (6.83 g, 39.2 mmol) to afford after workup and purification using method-AR, (S)-methyl 5-bromo-2-(1-(tert-butoxycarbonyl)pyrrolidin-3-yl)-2H-indazole-3-carboxylate (113b) (7.3 g, 88% yield) as a yellow oil; 1H NMR (300 MHz, DMSO-d₆) δ 8.20-8.16 (m, 1H), 7.81 (dd, J=9.1, 0.7 Hz, 1H), 7.51 (dd, J=9.1, 1.9 Hz, 1H), 6.19-6.04 (m, 1H), 4.00 (s, 3H), 3.89-3.78 (m, 1H), 3.72 (dd, J=11.8, 3.1 Hz, 1H), 3.62-3.40 (m, 2H), 2.49-2.33 (m, 2H), 1.40 (d, J=8.5 Hz, 9H); MS (ES+) 446.9 and 448.9 (M+Na); [α]_{D}=+15.51 [MeOH, 0.245].

### Step-2: Preparation of (S)-tert-butyl 3-(5-bromo-3-(hydroxymethyl)-2H-indazol-2-yl)pyrrolidine-1-carboxylate (113c)

Compound 113e was prepared according to the procedure and method of purification reported in step-2 of scheme-7 from (S)-methyl 5-bromo-2-(1-(tert-butoxycarbonyl)pyrrolidin-3-yl)-2H-indazole-3-carboxylate (113b) (7.3 g, 17.21 mmol) in DCM (50 mL) using DIBAL (1M solution in DCM 43.0 mL, 43.0 mmol) to afford after workup and purification using method-O, (S)-tert-butyl 3-(5-bromo-3-(hydroxymethyl)-2H-indazol-2-yl)pyrrolidine-1-carboxylate (113c) (3.9 g, 9.84 mmol, 57.2% yield) as clear oil; 1H NMR (300 MHz, DMSO-d₆) δ 8.04 (d, J=1.8 Hz, 1H), 7.57 (d, J=9.1 Hz, 1H), 7.31 (dd, J=9.1, 1.9 Hz, 1H), 5.54-5.34 (m, 2H), 4.93 (d, J=5.2 Hz, 2H), 3.87-3.76 (m, 1H), 3.71-3.55 (m, 2H), 3.51-3.38 (m, 1H), 2.46-2.29 (m, 2H), 1.41 (d, J=10.7 Hz, 9H).

### Step-3: Preparation of (S)-tert-butyl 3-(5-bromo-3-((2-(2-ethoxy-2-oxoethyl)phenoxy)methyl)-2H-indazol-2-yl)pyrrolidine-1-carboxylate (113d)

Compound 113d was prepared according to the procedure and method of purification reported in step-2 of scheme-2 from (S)-tert-butyl 3-(5-bromo-3-(hydroxymethyl)-2H-indazol-2-yl)pyrrolidine-1-carboxylate (113c) (3.9 g, 9.84 mmol) DCM (20 mL) using PPh₃ (2.84 g, 10.83 mmol), ethyl 2-(2-hydroxyphenyl)acetate (2b) (1.951 g, 10.83 mmol) and a solution of DCAD (3.98 g, 10.83 mmol) in DCM (20 mL) to afford after workup and purification using method-P, (S)-tert-butyl 3-(5-bromo-3-((2-(2-ethoxy-2-oxoethyl)phenoxy)methyl)-2H-indazol-2-yl)pyrrolidine-1-carboxylate (113d) (3.5 g, 63.7% yield) as yellow oil; MS (ES+): 558.1 and 560.1 (M+1).

### Step-4: Preparation of (S)-ethyl 2-(2-((5-bromo-2-(pyrrolidin-3-yl)-2H-indazol-3-yl)methoxy)phenyl)acetate (113e)

Compound 113e was prepared according to the procedure reported in step-4 of scheme-9 from (S)-tert-butyl 3-(5-bromo-3-((2-(2-ethoxy-2-oxoethyl)phenoxy)methyl)-2H-indazol-2-yl)pyrrolidine-1-carboxylate (113d) (3.5 g, 6.27 mmol) in DCM (20 mL) using TFA (2.414 mL, 31.3 mmol) to afford after workup and purification using method-P, (S)-ethyl 2-(2-((5-bromo-2-(pyrrolidin-3-yl)-2H-indazol-3-yl)methoxy)phenyl)acetate (113e) (2 g, 69.6% yield) as yellow solid; MS (ES+): 458.1 and 460.1 (M+1); (ES−): 456.0 and 458.1 (M−1).

### Step-5: Preparation of (S)-ethyl 2-(2-((5-bromo-2-(1-(2-hydroxyethyl)pyrrolidin-3-yl)-2H-indazol-3-yl)methoxy)phenyl)acetate (113f)

Compound 113f was prepared according to the procedure reported in step-3 of scheme-106 from (S)-ethyl 2-(2-((5-bromo-2-(pyrrolidin-3-yl)-2H-indazol-3-yl)methoxy)phenyl)acetate (113e) (500 mg, 1.091 mmol) in ACN (10 mL) using 2-bromoethanol (164 mg, 1.309 mmol) and K₂CO₃ (377 mg, 2.73 mmol) to afford after workup and purification using method-P, (S)-ethyl 2-(2-((5-bromo-2-(1-(2-hydroxyethyl)pyrrolidin-3-yl)-2H-indazol-3-yl)methoxy)phenyl)acetate (113f) (250 mg, 45.6% yield) as yellow oil; MS (ES+): 502.1 and 504.1 (M+1).

### Step-6: Preparation of (S)-ethyl 2-(2-((5-(1-aminoisoquinolin-7-yl)-2-(1-(2-hydroxyethyl)pyrrolidin-3-yl)-2H-indazol-3-yl)methoxy)phenyl)acetate (113g)

Compound 113g was prepared according to the procedure reported in step-3 of scheme-112 from (S)-ethyl 2-(2-((5-bromo-2-(1-(2-hydroxyethyl)pyrrolidin-3-yl)-2H-indazol-3-yl)methoxy)phenyl)acetate (113f) (125 mg, 0.249 mmol) in dioxane/2-Me THF (4 mL, 3:1) using 7-(4,4,5,5-tetramethyl-1.3,2-dioxaborolan-2-yl)isoquinolin-1-amine (9h). K₃PO₄ (2M aqueous solution, 0.498 mL, 0.995 mmol), PCy₃ (13.95 mg, 0.050 mmol) and Pd₂(dba)₃ (22.78 mg, 0.025 mmol) to afford after workup and purification using method-U, (S)-ethyl 2-(2-((5-(1-aminoisoquinolin-7-yl)-2-(1-(2-hydroxyethyl)pyrrolidin-3-yl)-2H-indazol-3-yl)methoxy)phenyl)acetate (113g) (113 mg, 80% yield) as a clear oil; MS (ES+): 566.2 (M+1).

### Step-7: Preparation of (S)-2-(2-((5-(1-aminoisoquinolin-7-yl)-2-(1-(2-hydroxyethyl)pyrrolidin-3-yl)-2H-indazol-3-yl)methoxy)phenyl)acetic acid (113h)

Compound 113h was prepared according to the procedure reported step-2 of scheme-1 from (S)-ethyl 2-(2-((5-(1-aminoisoquinolin-7-yl)-2-(1-(2-hydroxyethyl)pyrrolidin-3-yl)-2H-indazol-3-yl)methoxy)phenyl)acetate (113g) (113 mg, 0.200 mmol) in THF (3 mL) using a solution of LiOH·H₂O (17.88 mg, 0.746 mmol) in water (1 mL) to afford after workup and purification using method-M. (S)-2-(2-((5-(1-aminoisoquinolin-7-yl)-2-(1-(2-hydroxyethyl)pyrrolidin-3-yl)-2H-indazol-3-yl)methoxy)phenyl)acetic acid (113h) (57 mg, 42.6% yield) HCl salt as a white solid; 1H NMR (300 MHz, DMSO-d₆) δ 13.60 (s, 1H, DO exchangeable), 11.09 (s, 1H, D₂O exchangeable), 9.73-9.31 (m, 2H, D₂O exchangeable), 9.16-9.06 (m, 1H), 8.65-8.54 (m, 1H), 8.44 (dd, J=8.5, 1.6 Hz, 1H), 8.08-7.94 (m, 2H), 7.88-7.78 (m, 1H), 7.71 (dd, J=12.0, 6.9 Hz, 1H), 7.41-7.28 (m, 2H), 7.25 (d, J=7.0 Hz, 2H), 6.97 (td, J=7.1, 1.7 Hz, 1H), 5.85-5.59 (m, 3H), 4.36-4.15 (m, 1H), 3.92-3.73 (m, 4H), 3.69-3.60 (m, 2H), 3.50-3.26 (m, 4H), 2.81-2.56 (m, 1H), 2.48-2.32 (m, 1H); MS (ES+): 538.2 (M+1); (ES−): 536.2 (M−1).

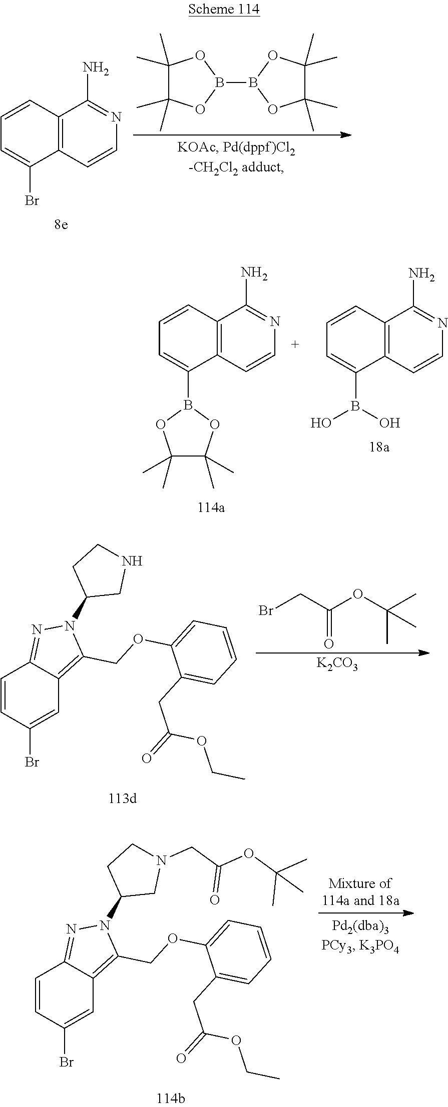

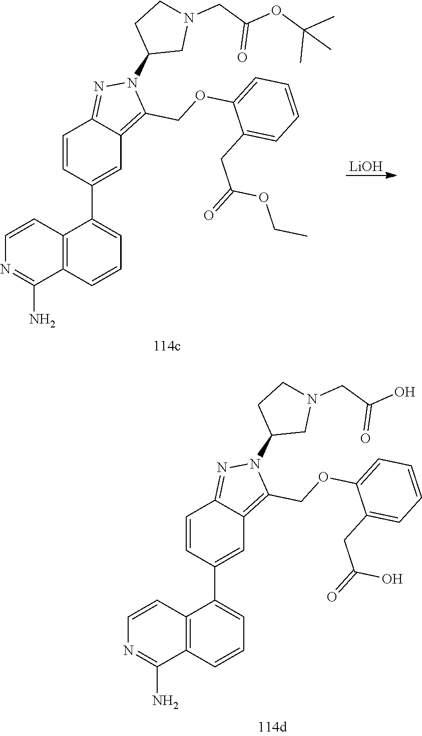 [SMILES: CCOC(=O)Cc1ccccc1OCc1c2cc(-c3cccc4c(N)nccc34)ccc2nn1[C@H]1CCN(CC(=O)OC(C)(C)C)C1 ; "LiOH" ; Nc1nccc2c(-c3ccc4nn([C@H]5CCN(CC(=O)O)C5)c(COc5ccccc5CC(=O)O)c4c3)cccc12]

### Preparation of (S)-2-(2-((5-(1-aminoisoquinolin-5-yl)-2-(1-(carboxymethyl)pyrrolidin-3-yl)-2H-indazol-3-yl)methoxy)phenyl)acetic acid (114d)

### Step-1: Preparation of 5-(4,4,5,5-tetramethyl-1,3,2-dioxaborolan-2-yl)isoquinolin-1-amine (114a)

Compound 114a was prepared according to the procedure reported in step-4 of scheme-1 from 5-bromoisoquinolin-1-amine (8e) (5 g, 22.41 mmol) in anhydrous dioxane (100 mL) using BISPIN (11.38 g, 44.8 mmol) KOAc (5.50 g, 56.0 mmol) and PdCl₂(dppf)-CH₂Cl₂ adduct (1.098 g, 1.345 mmol) to afford after workup and purification using method-Z, 3.6 g white solid as a mixture of 1-aminoisoquinolin-5-ylboronic acid (18a); MS (ES+): 189.1 (M+1), and 5-(4,4,5,5-tetramethyl-1,3,2-dioxaborolan-2-yl)isoquinolin-1-amine (114a); MS (ES+): 271.1 (M+1).

### Step-2: Preparation of (S)-tert-butyl 2-(3-(5-bromo-3-((2-(2-ethoxy-2-oxoethyl)phenoxy)methyl)-2H-indazol-2-yl)pyrrolidin-1-yl)acetate (114b)

Compound 114b was prepared according to the procedure reported in step-3 of scheme-106 from (S)-ethyl 2-(2-((5-bromo-2-(pyrrolidin-3-yl)-2H-indazol-3-yl)methoxy)phenyl)acetate (113e) (500 mg, 1.091 mmol) in ACN (10 mL) using tert-butyl 2-bromoacetate (255 mg, 1.309 mmol) and K₂CO₃ (377 mg, 2.73 mmol) to afford after workup and purification using method-P. (S)-tert-butyl 2-(3-(5-bromo-3-((2-(2-ethoxy-2-oxoethyl)phenoxy)methyl)-2H-indazol-2-yl)pyrrolidin-1-yl)acetate (114b) (132 mg, 21.14% yield) as yellow solid; MS (ES+): 572.1 and 574.2 (M+1).

### Step-3: Preparation of (S)-tert-butyl 2-(3-(5-(1-aminoisoquinolin-5-yl)-3-((2-(2-ethoxy-2-oxoethyl)phenoxy)methyl)-2H-indazol-2-yl)pyrrolidin-1-yl)acetate (114c)

Compound 114c was prepared according to the procedure reported in step-3 of scheme-112 from (S)-tert-butyl 2-(3-(5-bromo-3-((2-(2-ethoxy-2-oxoethyl)phenoxy)methyl)-2H-indazol-2-yl)pyrrolidin-1-yl)acetate (114b) (130 mg, 0.227 mmol) in dioxane/2-MeTHF (4 mL, 3:1) using a mixture of 5-(4,4,5,5-tetramethyl-1,3,2-dioxaborolan-2-yl)isoquinolin-1-amine (114a) and 1-aminoisoquinolin-5-ylboronic acid (18a) (92 mg), K₃PO₄ (2M aqueous solution, 0.170 mL, 0.681 mmol), PCy₃ (25.5 mg, 0.091 mmol) and Pd₂(dba)₃ (41.6 mg, 0.045 mmol) to afford after workup and purification using method-U, (S)-tert-butyl 2-(3-(5-(1-aminoisoquinolin-5-yl)-3-((2-(2-ethoxy-2-oxoethyl)phenoxy)methyl)-2H-indazol-2-yl)pyrrolidin-1-yl)acetate (114c) (68 mg, 47.1% yield) as a clear oil; MS (ES+): 636.3 (M+1).

### Step-4: Preparation of (S)-2-(2-((5-(1-aminoisoquinolin-5-yl)-2-(1-(carboxymethyl)pyrrolidin-3-yl)-2H-indazol-3-yl)methoxy)phenyl)acetic acid (114d)

Compound 114d was prepared according to the procedure reported step-2 of scheme-1 from (S)-tert-butyl 2-(3-(5-(1-aminoisoquinolin-5-yl)-3-((2-(2-ethoxy-2-oxoethyl)phenoxy)methyl)-2H-indazol-2-yl)pyrrolidin-1-ylacetate (114c) (68 mg, 0.107 mmol) in THF (3 mL) using a solution of LiOH·H₂O (38.1 mg, 1.59 mmol) in water (1 mL) to afford after workup and purification using method-M, (S)-2-(2-((5-(1-aminoisoquinolin-5-yl)-2-(1-(carboxymethyl)pyrrolidin-3-yl)-2H-indazol-3-yl)methoxy)phenyl)acetic acid (114d) (22 mg, 17.56% yield) HCl salt as a white solid; ¹H NMR (300 MHz, DMSO-d₆) δ 13.67 (s, 1H, D₂O exchangeable), 11.67 (m, 1H, 1H, D₂O exchangeable), 9.39 (s, 2H, 1H, D₂O exchangeable), 8.71 (d, J=8.4 Hz, 1H), 8.06 (s, 1H), 7.96 (d, J=7.2 Hz, 1H), 7.87 (t, J=8.4 Hz, 2H), 7.68 (d, J=7.1 Hz, 1H), 7.40 (dd, J=8.9, 1.6 Hz, 1H), 7.34-7.27 (m, 2H), 7.23 (d, J=7.4 Hz, 1H), 7.03-6.90 (m, 2H), 5.84-5.73 (m, 1H), 5.70 (s, 2H), 4.50-4.31 (m, 2H), 3.51 (s, 4H), 3.45 (s, 2H), 2.84-2.64 (m, 1H), 2.49-2.39 (m, 1H); MS (ES+) 552.2 (M+1); (ES−) 550.2 (M−1).

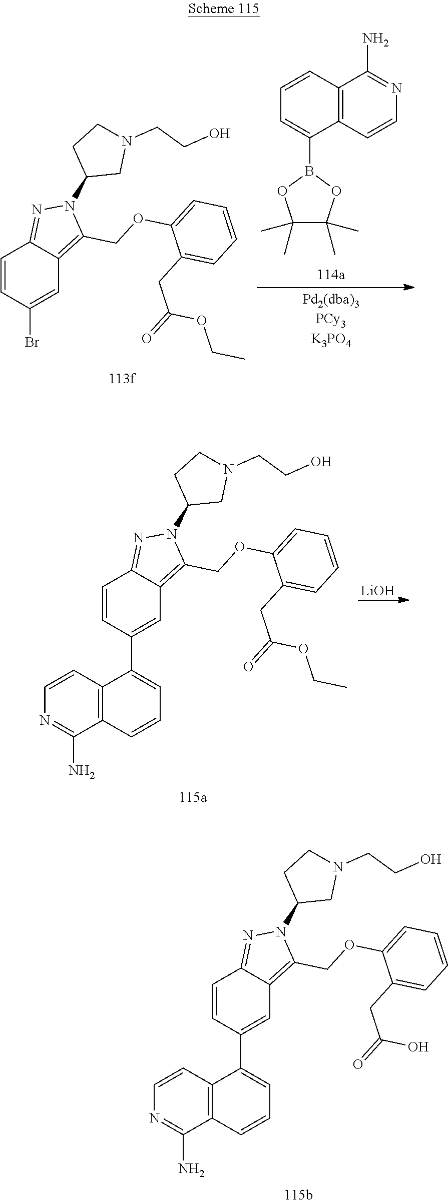

### Preparation of (S)-2-(2-((5-(1-aminoisoquinolin-5-yl)-2-(1-(2-hydroxyethyl)pyrrolidin-3-yl)-2H-indazol-3-yl)methoxy)phenyl)acetic acid (115b)

### Step-1: Preparation of (S)-ethyl 2-(2-((5-(1-aminoisoquinolin-5-yl)-2-(1-(2-hydroxyethyl)pyrrolidin-3-yl)-2H-indazol-3-yl)methoxy)phenyl)acetate (115a)

Compound 115a was prepared according to the procedure reported in step-3 of scheme-112 from (S)-ethyl 2-(2-((5-bromo-2-(1-(2-hydroxyethyl)pyrrolidin-3-yl)-2H-indazol-3-yl)methoxy)phenyl)acetate (113f) (125 mg, 0.249 mmol) in dioxane/2-MeTHF (4 mL, 3:1) using 5-(4,4,5,5-tetramethyl-1,3,2-dioxaborolan-2-yl)isoquinolin-1-amine (114a) (101 mg, 0.373 mmol), K₃PO₄ (2M aqueous solution, 0.187 mL, 0.746 mmol), PCy₃ (20.93 mg, 0.075 mmol) and Pd₂(dba)₃ (45.6 mg, 0.050 mmol) to afford after workup and purification using method-U, (S)-ethyl 2-(2-((5-(1-aminoisoquinolin-5-yl)-2-(1-(2-hydroxyethyl)pyrrolidin-3-yl)-2H-indazol-3-yl)methoxy)phenyl)acetate (115a) (56 mg, 39.8% yield) as a colorless oil; MS (ES+): 566.3 (M+1).

### Step-2: Preparation of (S)-2-(2-((5-(1-aminoisoquinolin-5-yl)-2-(1-(2-hydroxyethyl)pyrrolidin-3-yl)-2H-indazol-3-yl)methoxy)phenyl)acetic acid (115b)

Compound 115b was prepared according to the procedure reported step-2 of scheme-1 from (S)-ethyl 2-(2-((5-(1-aminoisoquinolin-5-yl)-2-(1-(2-hydroxyethyl)pyrrolidin-3-yl)-2H-indazol-3-yl)methoxy)phenyl)acetate (115a) (56 mg, 0.099 mmol) in THF (3 mL) using a solution of LiOH·H₂O (7.4 mgs, 0.297 mmol) in water (I mL) to afford after workup and purification using method-M, (S)-2-(2-((5-(1-aminoisoquinolin-5-yl)-2-(1-(2-hydroxyethyl)pyrrolidin-3-yl)-2H-indazol-3-yl)methoxy)phenyl)acetic acid (115b) (23 mg, 43.43% yield) HCl salt as a white solid; 1H NMR (300 MHz, DMSO-d₆) δ 13.74 (s, 1H, D₂O exchangeable), 10.91 (m, 1H), 9.57 (s, 2H, D₂O exchangeable), 8.73 (d, J=8.1 Hz, 1H), 8.04 (s, 1H), 7.95 (d, J=7.2 Hz, 1H), 7.91-7.77 (m, 2H), 7.67 (d, J=7.2 Hz, 1H), 7.37 (dd, J=8.9, 2.0 Hz, 1H), 7.29 (d, J=4.0 Hz, 2H), 7.22 (d, J=7.3 Hz, 1H), 7.00-6.89 (m, 2H), 5.87-5.60 (m, 3H), 4.33-4.02 (m, 2H), 3.88-3.78 (m, 4H), 3.72-3.53 (m, 4H), 2.85-2.58 (m, 1H), 2.46-2.32 (m, 1H); MS (ES+): 538.2 (M+1); (ES−): 536.2 (M−1).

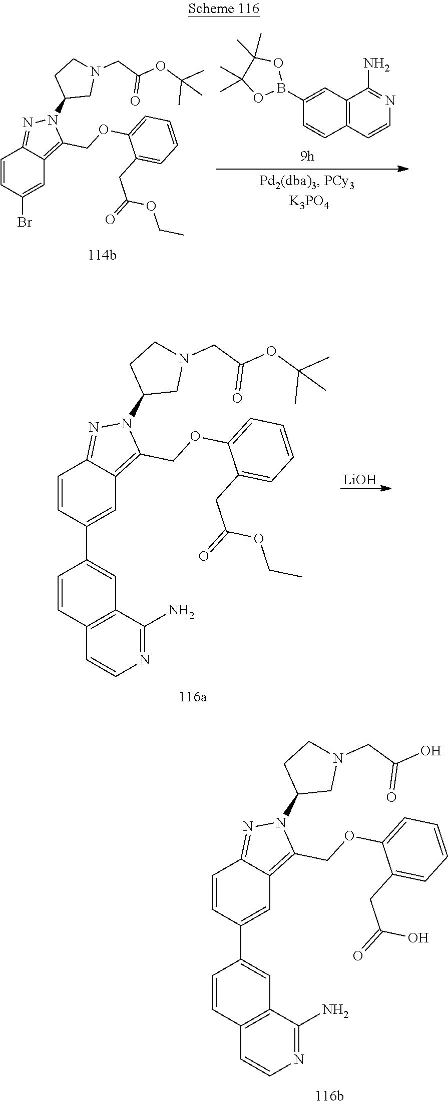

### Preparation of (S)-2-(2-((5-(1-aminoisoquinolin-7-yl)-2-(1-(carboxymethyl)pyrrolidin-3-yl)-2H-indazol-3-yl)methoxy)phenyl)acetic acid (116b)

### Step-1, Preparation of (S)-tert-butyl 2-(3-(5-(1-aminoisoquinolin-7-yl)-3-((2-(2-ethoxy-2-oxoethyl)phenoxy)methyl)-2H-indazol-2-yl)pyrrolidin-1-yl)acetate (116a)

Compound 116a was prepared according to the procedure reported in step-3 of scheme-112 from (S)-tert-butyl 2-(3-(5-bromo-3-((2-(2-ethoxy-2-oxoethyl)phenoxy)methyl)-2H-indazol-2-yl)pyrrolidin-1-yl)acetate (114b) (130 mg, 0.227 mmol) in dioxane/2-MeTHF (4 mL, 3:1) using 7-(4,4,5,5-tetramethyl-1,3,2-dioxaborolan-2-yl)isoquinolin-1-amine (9h) (92 mg, 0.341 mmol), K₃PO₄ (2M aqueous solution, 0.170 mL, 0.681 mmol), PCy₃ (25.5 mg, 0.091 mmol) and Pd₂(dba)₃ (41.6 mg, 0.045 mmol) to afford after workup and purification using method-U, (S)-tert-butyl 2-(3-(5-(1-aminoisoquinolin-7-yl)-3-((2-(2-ethoxy-2-oxoethyl)phenoxy)methyl)-2H-indazol-2-yl)pyrrolidin-1-yl)acetate (116a) (86 mg, 59.6% yield) as a clear oil; MS (ES+): 636.3 (M+1).

### Step-2, Preparation of (S)-2-(2-((5-(1-aminoisoquinolin-7-yl)-2-(1-(carboxymethyl)pyrrolidin-3-yl)-2H-indazol-3-yl)methoxy)phenyl)acetic acid (116b)

Compound 116b was prepared according to the procedure reported step-2 of scheme-1 from (S)-tert-butyl 2-(3-(5-(1-aminoisoquinolin-7-yl)-3-((2-(2-ethoxy-2-oxoethyl)phenoxy)methyl)-2H-indazol-2-yl)pyrrolidin-1-yl)acetate (116a) (86 mg, 0.135 mmol) in THF (3 mL) using a solution of LiOH·H₂O (27.2 mg, 1.135 mmol) in water (1 mL) to afford after workup and purification using method-M, (S)-2-(2-((5-(1-aminoisoquinolin-7-yl)-2-(1-(carboxymethyl)pyrrolidin-3-yl)-2H-indazol-3-yl)methoxy)phenyl)acetic acid (116b) (31 mg, 24.75% yield) HCl salt as a white solid; 1H NMR. (300 MHz, DMSO-d₆) δ 12.52 (brs, 2H, D₂O exchangeable), 9.36 (s, 2H, D₂O exchangeable), 9.09 (s, 1H), 8.60 (s, 1H), 8.45 (dd, J=8.6, 1.6 Hz, 1H), 8.09-7.95 (m, 2H), 7.86 (d, J=9.1 Hz, 1H), 7.70 (d, J=6.9 Hz, 1H), 7.40-7.29 (m, 2H), 7.29-7.20 (m, 2H), 7.02-6.92 (m, 1H), 5.87-5.59 (m, 3H), 4.45-4.20 (m, 2H), 4.11-3.84 (m, 2H), 3.79-3.60 (m, 2H), 3.52 (s, 2H), 2.79-2.63 (m, 1H), 2.48-2.36 (m, 1H); MS (ES+): 552.2 (M+1); (ES−): 550.1 (M−1).

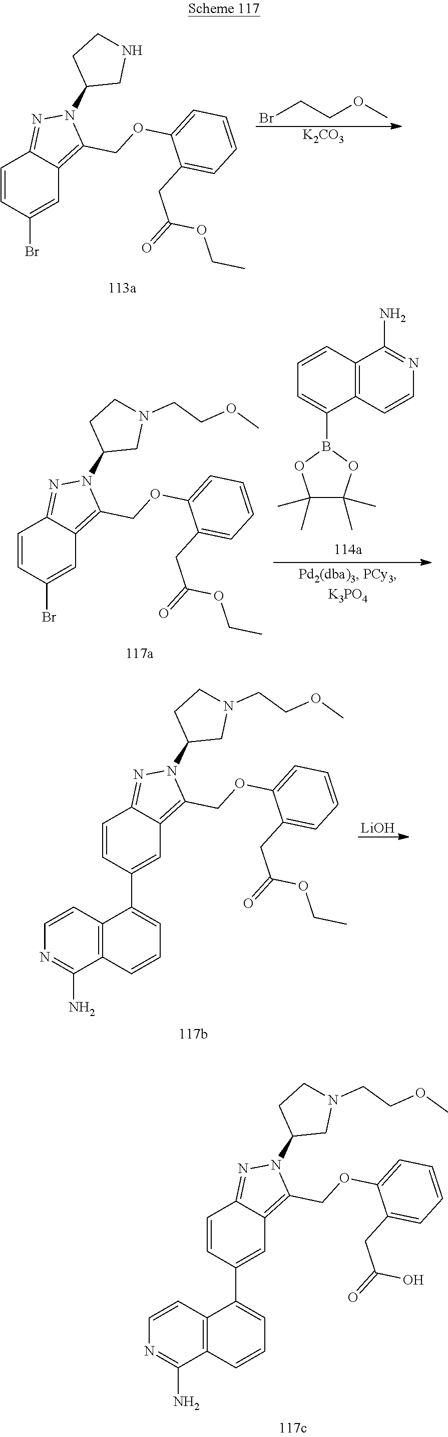

### Preparation of (S)-2-(2-((5-(1-aminoisoquinolin-5-yl)-2-(1-(2-methoxyethyl)pyrrolidin-3-yl)-2H-indazol-3-yl)methoxy)phenyl)acetic acid (117c)

### Step-1: Preparation of (S)-ethyl 2-(2-((5-bromo-2-(1-(2-methoxyethyl)pyrrolidin-3-yl)-2H-indazol-3-yl)methoxy)phenyl)acetate (117a)

Compound 117a was prepared according to the procedure reported in step-3 of scheme-106 from (S)-ethyl 2-(2-((5-bromo-2-(pyrrolidin-3-yl)-2H-indazol-3-yl)methoxy)phenyl)acetate (113e) (500 mg, 1.091 mmol) in ACN (10 mL) using 1-bromo-2-methoxyethane (182 mg, 1.309 mmol) and K₂CO₃ (377 mg, 2.73 mmol) to afford after workup and purification using method-P, (S)-ethyl 2-(2-((5-bromo-2-(1-(2-methoxyethyl)pyrrolidin-3-yl)-2H-indazol-3-yl)methoxy)phenyl)acetate (117a) (130 mg, 23.08% yield) as yellow solid; MS (ES+): 516.1 and 518.1 (M+1).

### Step-2: Preparation of (S)-ethyl 2-(2-((5-(1-aminoisoquinolin-5-yl)-2-(1-(2-methoxyethyl)pyrrolidin-3-yl)-2H-indazol-3-yl)methoxy)phenyl)acetate (117b)

Compound 117b was prepared according to the procedure reported in step-3 of scheme-112 from (S)-ethyl 2-(2-((5-bromo-2-(1-(2-methoxyethyl)pyrrolidin-3-yl)-2H-indazol-3-yl)methoxy)phenyl)acetate (117a) (130 mg, 0.252 mmol) in dioxane/2-MeTHF (4 mL, 3:1) using 5-(4,4,5,5-tetramethyl-1,3,2-dioxaborolan-2-yl)isoquinolin-1-amine (114a) (102 mg, 0.378 mmol), K₃PO₄ (2M aqueous solution, 0.189 mL, 0.755 mmol), PCy₃ (28.2 mg, 0.101 mmol) and Pd₂(dba)₃ (46.1 mg, 0.05 mmol) to afford after workup and purification using method-U. (S)-ethyl 2-(2-((5-(1-aminoisoquinolin-5-yl)-2-(1-(2-methoxyethyl)pyrrolidin-3-yl)-2H-indazol-3-yl)methoxy)phenyl)acetate (117b) (78 mg, 53.5% yield) as a clear oil; MS (ES+): 580.3 (M+1).

### Step-3: Preparation of (S)-2-(2-((5-(1-aminoisoquinolin-5-yl)-2-(1-(2-methoxyethyl)pyrrolidin-3-yl)-2H-indazol-3-yl)methoxy)phenyl)acetic acid (117c)

Compound 117c was prepared according to the procedure reported step-2 of scheme-1 from (S)-ethyl 2-(2-((5-(1-aminoisoquinolin-5-yl)-2-(1-(2-methoxyethyl)pyrrolidin-3-yl)-2H-indazol-3-yl)methoxy)phenyl)acetate (117b) (78 mg, 0.135 mmol, 53.5% yield) in THF (3 mL) using a solution of LiOH·H₂O (18.09 mg, 0.755 mmol) in water (1 mL) to afford after workup and purification using method-M, (S)-2-(2-((5-(1-aminoisoquinolin-5-yl)-2-(1-(2-methoxyethyl)pyrrolidin-3-yl)-2H-indazol-3-yl)methoxy)phenyl)acetic acid (117c) (30 mg, 21.60% yield) HCl salt as a white solid; ¹H NMR (300 MHz, DMSO-d₆) δ 13.51 (s, 1H), 12.16 (s, 1H), 10.85 (d, J=154.5 Hz, 1H), 9.28 (s, 2H), 8.67 (d, J=8.3 Hz, 1H), 8.05 (s, 1H), 7.96 (d, J=7.2 Hz, 1H), 7.91-7.80 (m, 2H), 7.67 (d, J=7.2 Hz, 1H), 7.40 (dd, J=8.9, 1.6 Hz, 1H), 7.33-7.27 (m, 2H), 7.24 (d, J=7.4 Hz, 1H), 7.02-6.92 (m, 2H), 5.83-5.56 (m, 3H), 4.31-3.92 (m, 2H), 3.75 (t, J=4.8 Hz, 4H), 3.61 (d, J=18.7 Hz, 2H), 3.51 (s, 2H), 3.34 (s, 3H), 2.55 (s, 2H); MS (ES+) 552.3 (M+1); (ES−) 550.2 (M−1).

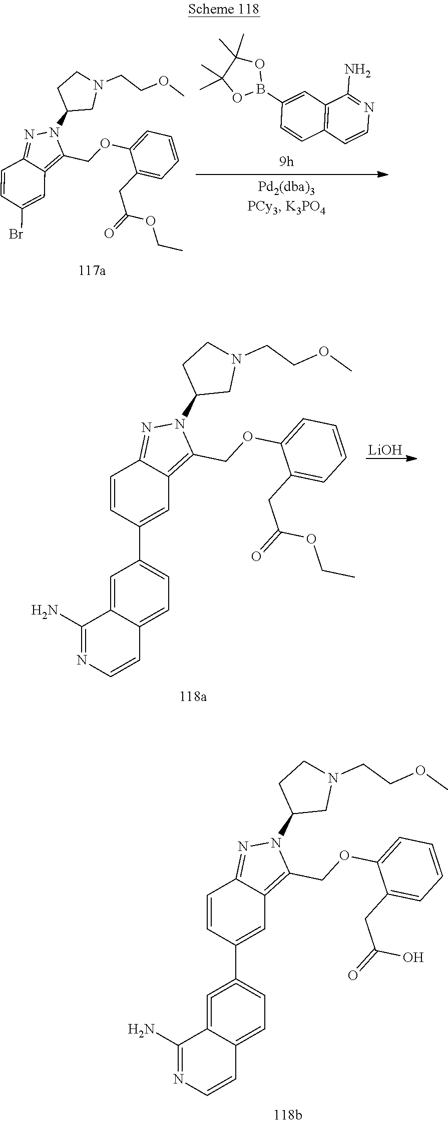

### Preparation of (S)-2-(2-((5-(1-aminoisoquinolin-7-yl)-2-(1-(2-methoxyethyl)pyrrolidin-3-yl)-2H-indazol-3-yl)methoxy)phenyl)acetic acid (118b)

### Step-1: Preparation of (S)-ethyl 2-(2-((5-(1-aminoisoquinolin-7-yl)-2-(1-(2-methoxyethyl)pyrrolidin-3-yl)-2H-indazol-3-yl)methoxy)phenyl)acetate (118a)

Compound 118a was prepared according to the procedure reported in step-3 of scheme-112 from (S)-ethyl 2-(2-((5-bromo-2-(1-(2-methoxyethyl)pyrrolidin-3-yl)-2H-indazol-3-yl)methoxy)phenyl)acetate (117a) (130 mg, 0.252 mmol) in dioxane/2-MeTHF (4 mL, Ratio: 3:1) using 7-(4,4,5,5-tetramethyl-1,3,2-dioxaborolan-2-yl)isoquinolin-1-amine (9h) (102 mg, 0.378 mmol), K₃PO₄ (2M aqueous solution, 0.189 mL, 0.755 mmol), PCy₃ (28.2 mg, 0.101 mmol) and Pd₂(dba)₃ (46.1 mg, 0.050 mmol) to afford after workup and purification using method-U, (S)-ethyl 2-(2-((5-(1-aminoisoquinolin-7-yl)-2-(1-(2-methoxyethyl)pyrrolidin-3-yl)-2H-indazol-3-yl)methoxy)phenyl)acetate (118a) (92 mg, 63.0% yield) as a clear oil; MS (ES+): 580.3 (M+1).

### Step-2: Preparation of (S)-2-(2-((5-(1-aminoisoquinolin-7-yl)-2-(1-(2-methoxyethyl)pyrrolidin-3-yl)-2H-indazol-3-yl)methoxy)phenyl)acetic acid (118b)

Compound 118b was prepared according to the procedure reported step-2 of scheme-1 from (S)-ethyl 2-(2-((5-(1-aminoisoquinolin-7-yl)-2-(1-(2-methoxyethyl)pyrrolidin-3-yl)-2H-indazol-3-yl)methoxy)phenyl)acetate (118a) (92 mg, 0.159 mmol) in THF (3 mL) using a solution of LiOH·H₂O (30.1 mg, 1.259 mmol) in water (1 mL) to afford after workup and purification using method-M. (S)-2-(2-((5-(1-aminoisoquinolin-7-yl)-2-(1-(2-methoxyethyl)pyrrolidin-3-yl)-2H-indazol-3-yl)methoxy)phenyl)acetic acid (118b) (46 mg, 33.1% yield) HCl salt as a white solid; 1H NMR (300 MHz, DMSO-d₆+D₂O) δ 8.96 (s, 1H), 8.47 (s, 1H), 8.43 (dd, J=8.5, 1.8 Hz, 1H), 8.05 (d, J=8.5 Hz, 1H), 7.98-7.92 (m, 1H), 7.90-7.83 (m, 1H), 7.65 (d, J=6.9 Hz, 1H), 7.34 (d, J=4.1 Hz, 2H), 7.31-7.21 (m, 2H), 7.04-6.94 (m, 1H), 5.87-5.58 (m, 3H), 4.26-3.89 (m, 2H), 3.82-3.69 (m, 6H), 3.51 (s, 2H), 3.34 (s, 3H), 2.50-2.37 (m, 2H); MS (ES+): 552.3 (M+1); (ES−): 550.2 (M−1).

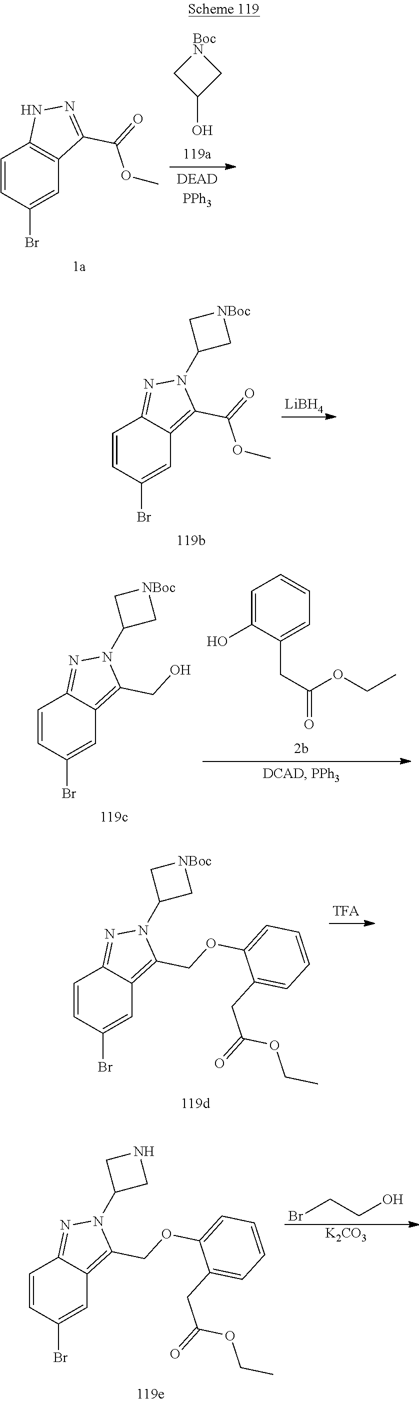

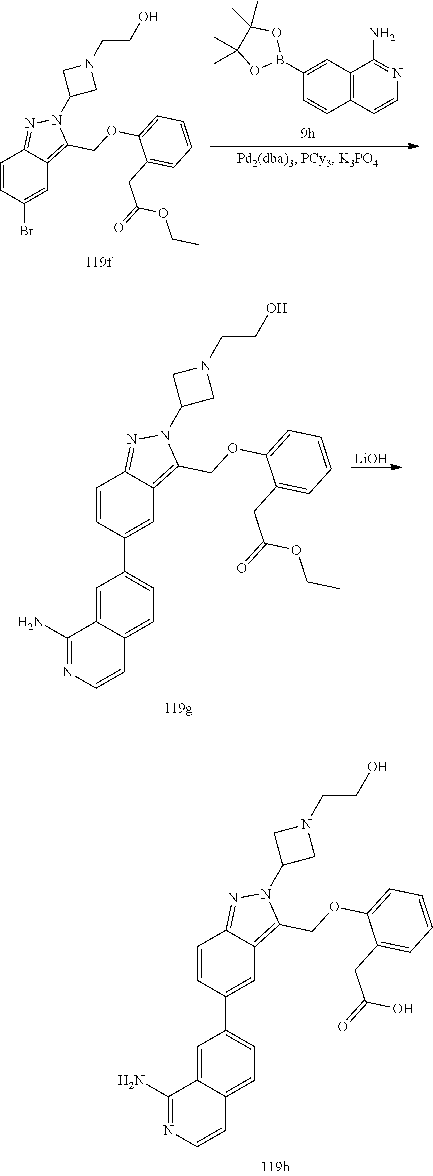

### Preparation of 2-(2-((5-(1-aminoisoquinolin-7-yl)-2-(1-(2-hydroxyethyl)azetidin-3-yl)-2H-indazol-3-yl)methoxy)phenyl)acetic acid (119h)

### Step-1, Preparation of methyl 5-bromo-2-(1-(tert-butoxycarbonyl)azetidin-3-yl)-2H-indazole-3-carboxylate (119b)

Compound 119b was prepared according to the procedure reported in step-1 of scheme-8 from methyl 5-bromo-1H-indazole-3-carboxylate (1a) (10 g, 39.2 mmol) in THF using DEAD (13.66 g, 78 mmol), PPh₃ (20.57 g, 78 mmol) and tert-butyl 3-hydroxyazetidine-1-carboxylate (119a) (13.58 g, 78 mmol) to afford after workup and purification using method-AR, methyl 5-bromo-2-(1-(tert-butoxycarbonyl)azetidin-3-yl)-2H-indazole-3-carboxylate (119b) (12 g, 29.2 mmol, 74.6% yield) as yellow solid; 1H NMR (300 MHz, DMSO-d₆) δ 8.17 (dd, J=1.9, 0.8 Hz, 1H), 7.92-7.84 (m, 1H), 7.54 (dd, J=9.1, 1.9 Hz, 1H), 6.22-6.09 (m, 1H), 4.48-4.27 (m, 4H), 3.99 (s, 3H), 1.42 (s, 9H).

### Step-2: Preparation of tert-butyl 3-(5-bromo-3-(hydroxymethyl)-2H-indazol-2-yl)azetidine-1-carboxylate (119c)

To a cold solution of methyl 5-bromo-2-(1-(tert-butoxycarbonyl)azetidin-3-yl)-2H-indazole-3-carboxylate (119b) (6.4 g, 15.60 mmol) in THF (30 mL) was added LiBH₄ (4M in THF, 11.70 mL, 46.8 mmol) and MeOH (1.893 mL, 46.8 mmol) at −78° C. and allowed to warm to RT over 24 h. The reaction was quenched with NH₄Cl (sat.) and extracted with EtOAc, the combined organics were washed with brine, dried, filtered and concentrated. The residue obtained was purified using method-N, to give tert-butyl 3-(5-bromo-3-(hydroxymethyl)-2H-indazol-2-yl)azetidine-1-carboxylate (119c) (3 g, 50.3% yield) as a white solid; ¹H NMR (300 MHz, DMSO-d₆) δ 8.06 (dd, J=1.9, 0.8 Hz, 1H), 7.65 (dd, J=9.1, 0.8 Hz, 1H), 7.35 (dd, J=9.1, 1.9 Hz, 1H), 5.68-5.57 (m, 1H), 5.48 (t, J=5.6 Hz, 1H), 4.87 (d, J=5.6 Hz, 2H), 4.42-4.22 (m, 4H), 1.43 (s, 9H); MS (ES+): 404.0 and 406.0 (M+Na); (ES−): 416.0 and 418.0 (M+Cl).

### Step-3: Preparation of tert-butyl 3-(5-bromo-3-((2-(2-ethoxy-2-oxoethyl)phenoxy)methyl)-2H-indazol-2-yl)azetidine-1-carboxylate (119d)

Compound 119d was prepared according to the procedure reported in step-2 of scheme-2, from tert-butyl 3-(5-bromo-3-(hydroxymethyl)-2H-indazol-2-yl)azetidine-1-carboxylate (119c) (3 g, 7.85 mmol) in DCM (100 mL) using PPh₃ (2.264 g, 8.63 mmol), ethyl 2-(2-hydroxyphenyl)acetate (2b) (1.556 g, 8.63 mmol) and a solution of DCAD (3.17 g, 8.63 mmol) in DCM (20 mL) dropwise to afford after workup and purification using method-K, tert-butyl 3-(5-bromo-3-((2-(2-ethoxy-2-oxoethyl)phenoxy)methyl)-2H-indazol-2-yl)azetidine-1-carboxylate (119d) (3 g, 70.2% yield) as yellow oil; ¹H NMR (300 MHz. DMSO-d₆) δ 8.18 (d, J=1.8 Hz, 1H), 7.71 (d, J=9.2 Hz, 1H), 7.40 (dd, J=9.2, 1.8 Hz, 1H), 7.36-7.30 (m, 1H), 7.30-7.19 (m, 2H), 6.97 (td, J=7.3, 1.3 Hz, 1H), 5.74-5.61 (m, 1H), 5.57 (s, 2H), 4.48-4.25 (m, 4H), 3.79 (q, J=7.1 Hz, 2H), 3.52 (s, 2H), 1.43 (s, 9H), 0.89 (t, J=7.1 Hz, 3H); MS (ES+): 544.1 and 546.2 (M+1); 566.1 and 568.1 (M+Na); (ES−): 542.1 and 544.1 (M−1).

### Step-4: Preparation of ethyl 2-(2-((2-(azetidin-3-yl)-5-bromo-2H-indazol-3-yl)methoxy)phenyl)acetate (119e)

Compound 119e was prepared according to the procedure reported in step-4 of scheme-9 from tert-butyl 3-(5-bromo-3-((2-(2-ethoxy-2-oxoethyl)phenoxy)methyl)-2H-indazol-2-yl)azetidine-1-carboxylate (119d) (3 g, 5.51 mmol), in DCM (100 mL) using TFA (2.123 mL, 27.6 mmol) in DCM (20 mL) to obtain a suspension, which was filtered to remove the solid, the filtrated was concentrated and purified using method-K, to give ethyl 2-(2-((2-(azetidin-3-yl)-5-bromo-2H-indazol-3-yl)methoxy)phenyl)acetate (119e) (2 g, 4.50 mmol, 82% yield) as yellow oil, which was mixed with the solid and lyophilized with water (containing 0.1% HCl) and ACN to give 119e as a yellow solid; ¹H NMR (300 MHz, DMSO-d₆) δ 9.63 (d, J=27.5 Hz, 2H), 8.21 (d, J=1.9 Hz, 1H), 7.73 (d, J=9.1 Hz, 1H), 7.48-7.41 (m, 1H), 7.39-7.31 (m, 1H), 7.25 (td, J=7.8, 1.4 Hz, 2H), 6.98 (td, J=7.4, 1.2 Hz, 1H), 5.90-5.77 (m, 1H), 5.59 (s, 2H), 4.55-4.43 (m, 4H), 3.75 (q, J=7.1 Hz, 2H), 3.53 (s, 2H), 0.85 (t, J=7.1 Hz, 3H).

### Step-5: Preparation of ethyl 2-(2-((5-bromo-2-(1-(2-hydroxyethyl)azetidin-3-yl)-2H-indazol-3-yl)methoxy)phenyl)acetate (119f)

Compound 119f was prepared according to the procedure reported in step-3 of scheme-106 from ethyl 2-(2-((2-(azetidin-3-yl)-5-bromo-2H-indazol-3-yl)methoxy)phenyl)acetate (119e) (500 mg, 1.125 mmol) in ACN (10 mL) using 2-bromoethanol (169 mg, 1.350 mmol) and K₂CO₃ (389 mg, 2.81 mmol) to afford after workup and purification using method-P, ethyl 2-(2-((5-bromo-2-(1-(2-hydroxyethyl)azetidin-3-yl)-2H-indazol-3-yl)methoxy)phenyl)acetate (119f) (180 mg, 32.8% yield) as yellow oil; MS (ES+): 488.1 and 490.0 (M+1).

### Step-6: Preparation of ethyl 2-(2-((5-(1-aminoisoquinolin-7-yl)-2-(1-(2-hydroxyethyl)azetidin-3-yl)-2H-indazol-3-yl)methoxy)phenyl)acetate (119g)

Compound 119g was prepared according to the procedure reported in step-3 of scheme-112 from ethyl 2-(2-((5-bromo-2-(1-(2-hydroxyethyl)azetidin-3-yl)-2H-indazol-3-yl)methoxy)phenyl)acetate (119f) (90 mg, 0.184 mmol) in dioxane/2-MeTHF (4 mL, 3:1) using 7-(4,4,5,5-tetramethyl-1,3,2-dioxaborolan-2-yl)isoquinolin-1-amine (9h) (74.7 mg, 0.276 mmol), K₃PO₄ (2M aqueous solution, 0.138 mL, 0.553 mmol), PCy₃ (20.67 mg, 0.074 mmol) and Pd₂(dba)₃ (33.8 mg, 0.037 mmol) to afford after workup and purification using method-U, ethyl 2-(2-((5-(1-aminoisoquinolin-7-yl)-2-(1-(2-hydroxyethyl)azetidin-3-yl)-2H-indazol-3-yl)methoxy)phenyl)acetate (119g) (52 mg, 51.2% yield) as a clear oil; MS (ES+): 552.3 (M+1).

### Step-7: Preparation of 2-(2-((5-(1-aminoisoquinolin-7-yl)-2-(1-(2-hydroxyethyl)azetidin-3-yl)-2H-indazol-3-yl)methoxy)phenyl)acetic acid (119h)

Compound 119h was prepared according to the procedure reported step-2 of scheme-1 from ethyl 2-(2-((5-(1-aminoisoquinolin-7-yl)-2-(1-(2-hydroxyethyl)azetidin-3-yl)-2H-indazol-3-yl)methoxy)phenyl)acetate (119g) (52 mg, 0.094 mmol, 51.2% yield) in THF (3 mL) using a solution of LiOH·H₂O (13.24 mg, 0.553 mmol) in water (1 mL) to afford after workup and purification using method-M, 2-(2-((5-(1-aminoisoquinolin-7-yl)-2-(1-(2-hydroxyethyl)azetidin-3-yl)-2H-indazol-3-yl)methoxy)phenyl)acetic acid (119h) (20 mg, 20.73% yield) HCl salt as a white solid; ¹H NMR (300 MHz, DMSO-d₆) δ 13.32 (s, 1H, D₂O exchangeable), 12.13 (s, 1H, D₂O exchangeable), 10.62 (s, 1H, D₂O exchangeable), 9.23 (s, 1H, D₂O exchangeable), 9.05 (s, 1H), 8.57 (s, 1H), 8.46 (d, J=8.5 Hz, 1H), 8.10-7.98 (m, 2H), 7.93 (d, J=9.1 Hz, 1H), 7.71 (d, J=6.9 Hz, 1H), 7.40-7.31 (m, 2H), 7.31-7.23 (m, 2H), 7.05-6.94 (m, 1H), 5.94-5.75 (m, 1H), 5.71 (s, 2H), 5.37-5.24 (m, 1H, D₂O exchangeable), 4.91-4.58 (m, 4H), 3.80-3.68 (m, 2H), 3.61-3.47 (m, 4H); MS (ES+): 524.2 (M+1); (ES−): 522.2 (M−1).

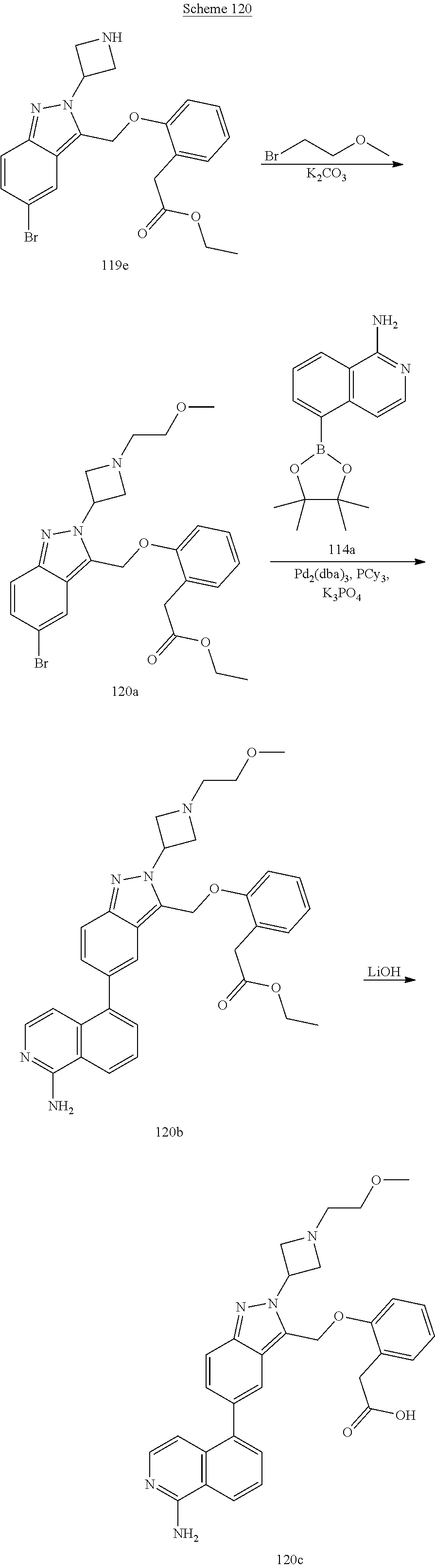

### Preparation of 2-(2-((5-(1-aminoisoquinolin-5-yl)-2-(1-(2-methoxyethyl)azetidin-3-yl)-2H-indazol-3-yl)methoxy)phenyl)acetic acid (120c)

### Step-1: Preparation of ethyl 2-(2-((5-bromo-2-(1-(2-methoxyethyl)azetidin-3-yl)-2H-indazol-3-yl)methoxy)phenyl)acetate (120a)

Compound 120a was prepared according to the procedure reported in step-3 of scheme-106 from ethyl 2-(2-((2-(azetidin-3-yl)-5-bromo-2H-indazol-3-yl)methoxy)phenyl)acetate (119e) (500 mg, 1.125 mmol) in ACN (10 mL) using 1-bromo-2-methoxyethane (188 mg, 1.350 mmol) and K₂CO₃ (389 mg, 2.81 mmol) to afford after workup and purification using method-P, ethyl 2-(2-((5-bromo-2-(1-(2-methoxyethyl)azetidin-3-yl)-2H-indazol-3-yl)methoxy)phenyl)acetate (120a) (260 mg, 46.0% yield) as yellow oil; ¹H NMR (300 MHz, DMSO-d₆) δ 8.14 (d, J=1.8 Hz, 1H), 7.69 (d, J=9.0 Hz, 1H), 7.42-7.18 (m, 4H), 7.04-6.91 (m, 1H), 5.55 (s, 2H), 5.47-5.35 (m, 1H), 3.89-3.76 (m, 4H), 3.64 (t, J=7.1 Hz, 2H), 3.51 (s, 2H), 3.38-3.35 (m, 2H), 3.23 (s, 3H), 2.70 (t, J=5.7 Hz, 2H), 0.91 (t, J=7.1 Hz, 3H); MS (ES+): 502.1 and 504.1 (M+1).

### Step-2: Preparation of ethyl 2-(2-((5-(1-aminoisoquinolin-5-yl)-2-(1-(2-methoxyethyl)azetidin-3-yl)-2H-indazol-3-yl)methoxy)phenyl)acetate (120b)

Compound 120b was prepared according to the procedure reported in step-3 of scheme-112 from ethyl 2-(2-((5-bromo-2-(1-(2-methoxyethyl)azetidin-3-yl)-2H-indazol-3-yl)methoxy)phenyl)acetate (120a) (100 mg, 0.199 mmol) in dioxane/2-MeTHF (4 mL, 3:1) using 5-(4,4,5,5-tetramethyl-1,3,2-dioxaborolan-2-yl)isoquinolin-1-amine (114a) (81 mg, 0.299 mmol), K₃PO₄ (2M aqueous solution, 0.149 mL, 0.597 mmol), PCy₃ (22.33 mg, 0.080 mmol) and Pd₂(dba)₃ (36.5 mg, 0.040 mmol) to afford after workup and purification using method-U, ethyl 2-(2-((5-(1-aminoisoquinolin-5-yl)-2-(1-(2-methoxyethyl)azetidin-3-yl)-2H-indazol-3-yl)methoxy)phenyl)acetate (120b) (58 mg, 51.5% yield) as a clear oil; MS (ES+): 566.3 (M+1).

### Step-3: Preparation of 2-(2-((5-(1-aminoisoquinolin-5-yl)-2-(1-(2-methoxyethyl)azetidin-3-yl)-2H-indazol-3-yl)methoxy)phenyl)acetic acid (120c)

Compound 120c was prepared according to the procedure reported step-2 of scheme-1 from ethyl 2-(2-((5-(1-aminoisoquinolin-5-yl)-2-(1-(2-methoxyethyl)azetidin-3-yl)-2H-indazol-3-yl)methoxy)phenyl)acetate (120b) (58 mg, 0.103 mmol) in THF (3 mL) using a solution of LiOH·H₂O (14.30 mg, 0.597 mmol) in water (1 mL) to afford after workup and purification using method-M, 2-(2-((5-(1-aminoisoquinolin-5-yl)-2-(1-(2-methoxyethyl)azetidin-3-yl)-2H-indazol-3-yl)methoxy)phenyl)acetic acid (120c) (22 mg, 20.56% yield) HCl salt as a white solid; 1H NMR (300 MHz, DMSO-d₆) δ 13.58 (s, 1H, D₂O exchangeable), 11.02 (d, J=64.0 Hz, 1H, D₂O exchangeable), 9.35 (s, 2H, D₂O exchangeable), 8.68 (d, J=8.3 Hz, 1H), 8.11-8.02 (m, 1H), 8.00-7.93 (m, 1H), 7.93-7.83 (m, 2H), 7.67 (d, J=7.2 Hz, 1H), 7.47-7.37 (m, 1H), 7.34-7.18 (m, 3H), 7.00-6.89 (m, 2H), 5.97-5.72 (m, 1H), 5.71-5.59 (m, 2H), 4.91-4.55 (m, 4H), 3.75-3.65 (m, 4H), 3.34-3.32 (m, 2H), 2.54 (s, 3H); MS (ES+): 538.3 (M+1); (ES−): 536.2 (M−1).

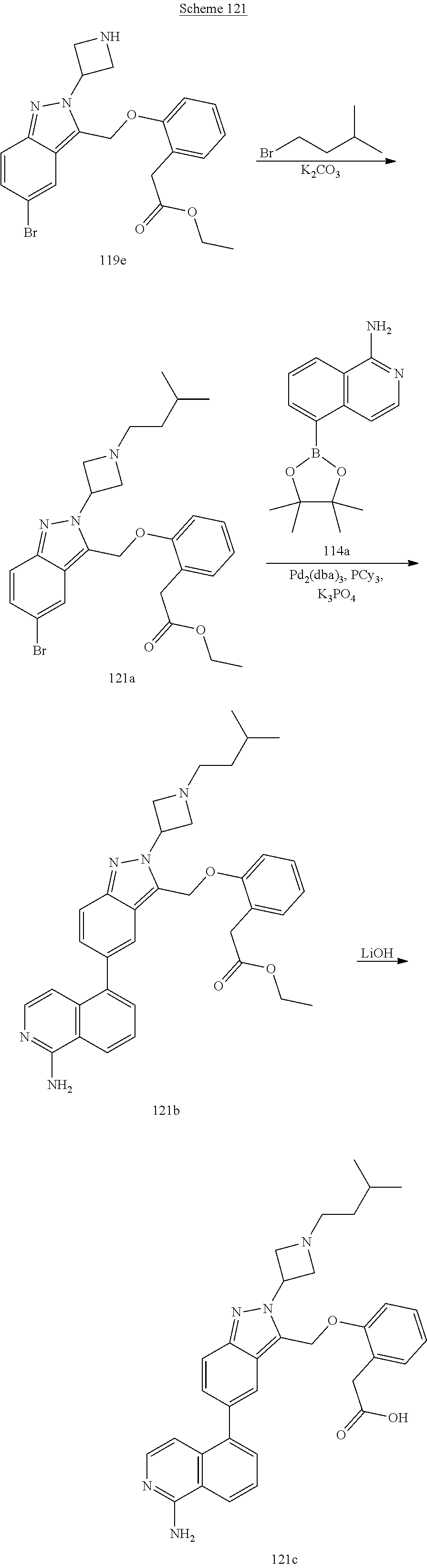

### Preparation of 2-(2-((5-(1-aminoisoquinolin-5-yl)-2-(1-isopentylazetidin-3-yl)-2H-indazol-3-yl)methoxy)phenyl)acetic acid (121c)

### Step-1: Preparation of ethyl 2-(2-((5-bromo-2-(1-isopentylazetidin-3-yl)-2H-indazol-3-yl)methoxy)phenyl)acetate (121a)

Compound 121a was prepared according to the procedure reported in step-3 of scheme-106 from ethyl 2-(2-((2-(azetidin-3-yl)-5-bromo-2H-indazol-3-yl)methoxy)phenyl)acetate (119e) (500 mg, 1.125 mmol) in ACN (10 mL) using 1-bromo-3-methylbutane (204 mg, 1.350 mmol) and K₂CO₃ (389 mg, 2.81 mmol) to afford after workup and purification using method-P, ethyl 2-(2-((5-bromo-2-(1-isopentylazetidin-3-yl)-2H-indazol-3-yl)methoxy)phenyl)acetate (121a) (278 mg, 48.0% yield) as yellow solid; MS (ES+): 514.2 and 516.1 (M+1); (ES−): 512.1 and 524.1 (M−1).

### Step-2: Preparation of ethyl 2-(2-((5-(1-aminoisoquinolin-5-yl)-2-(1-isopentylazetidin-3-yl)-2H-indazol-3-yl)methoxy)phenyl)acetate (121b)

Compound 121b was prepared according to the procedure reported in step-3 of scheme-112 from ethyl 2-(2-((5-bromo-2-(1-isopentylazetidin-3-yl)-2H-indazol-3-yl)methoxy)phenyl)acetate (121a) (135 mg, 0.262 mmol) in dioxane/2-MeTHF (4 mL, 3:1) using 5-(4,4,5,5-tetramethyl-1,3,2-dioxaborolan-2-yl)isoquinolin-1-amine (114a) (106 mg, 0.394 mmol), K₃PO₄ (2M aqueous solution, 0.197 mL, 0.787 mmol), PCy₃ (29.4 mg, 0.105 mmol) and Pd₂(dba)₃ (48.1 mg, 0.052 mmol) to afford after workup and purification using method-U, ethyl 2-(2-((5-(1-aminoisoquinolin-5-yl)-2-(1-isopentylazetidin-3-yl)-2H-indazol-3-yl)methoxy)phenyl)acetate (121b) (66 mg, 43.5% yield) as a clear oil; MS (ES+): 578.3 (M+1).

### Step-3: Preparation of 2-(2-((5-(1-aminoisoquinolin-5-yl)-2-(1-isopentylazetidin-3-yl)-2H-indazol-3-yl)methoxy)phenyl)acetic acid (121c)

Compound 121c was prepared according to the procedure reported step-2 of scheme-1 from ethyl 2-(2-((5-(1-aminoisoquinolin-5-yl)˜2-(1-isopentylazetidin-3-yl)-2H-indazol-3-yl)methoxy)phenyl)acetate (121b) (66 mg, 0.114 mmol) in THF (3 mL) using a solution of LiOH·H₂O (18.85 mg, 0.787 mmol) in water (1 mL) to afford after workup and purification using method-M, 2-(2-((5-(1-aminoisoquinolin-5-yl)-2-(1-isopentylazetidin-3-yl)-2H-indazol-3-yl)methoxy)phenyl)acetic acid (121c) (35 mg, 24.27% yield) HCl salt as a white solid; ¹H NMR (300 MHz, DMSO-d₆) δ 13.50 (s, 1H, D₂O exchangeable), 12.14 (s, 1H, D₂O exchangeable), 10.81 (s, 1H, D₂O exchangeable), 9.23 (s, 2H, D₂O exchangeable), 8.66 (d, J=8.3 Hz, 1H), 8.06 (s, 1H), 7.96 (d, J=7.2 Hz, 1H), 7.93-7.82 (m, 2H), 7.68 (d, J=7.2 Hz, 1H), 7.46-7.39 (m, 1H), 7.35-7.19 (m, 3H), 7.02-6.89 (m, 2H), 6.02-5.79 (m, 1H), 5.66 (s, 2H), 4.90-4.28 (m, 4H), 3.57-3.42 (m, 4H), 1.79-1.60 (m, 1H), 1.59-1.41 (m, 2H), 0.94 (d, J=6.5 Hz, 6H); MS (ES+): 550.3 (M+1); (ES−): 548.2 (M−1).

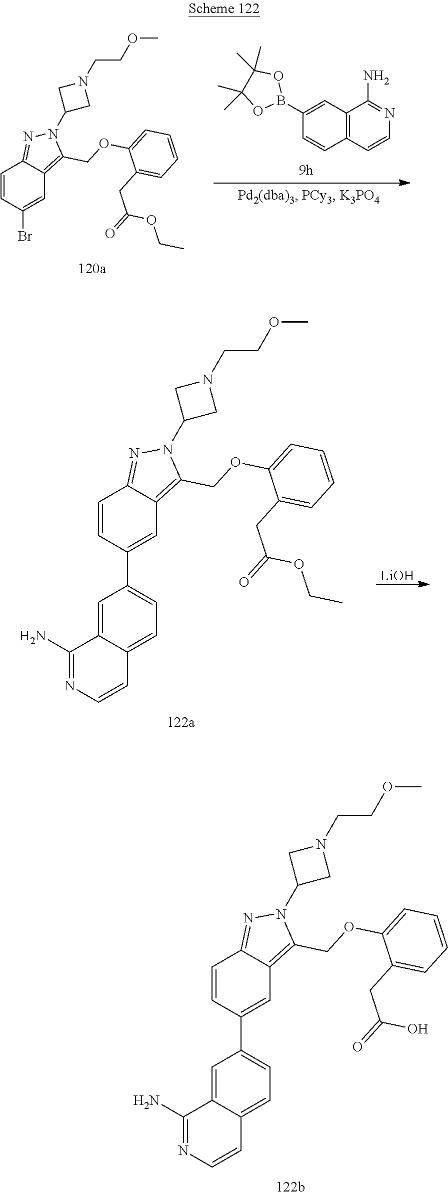

### Preparation of 2-(2-((5-(1-aminoisoquinolin-7-yl)-2-(1-(2-methoxyethyl)azetidin-3-yl)-2H-indazol-3-yl)methoxy)phenyl)acetic acid (122b)

### Step-1: Preparation of ethyl 2-(2-((5-(1-aminoisoquinolin-7-yl)-2-(1-(2-methoxyethyl)azetidin-3-yl)-2H-indazol-3-yl)methoxy)phenyl)acetate (122a)

Compound 122a was prepared according to the procedure reported in step-3 of scheme-112 from ethyl 2-(2-((5-bromo-2-(1-(2-methoxyethyl)azetidin-3-yl)-2H-indazol-3-yl)methoxy)phenyl)acetate (120a) (100 mg, 0.199 mmol) in dioxane/2-MeTHF (4 mL, 3:1) using 7-(4,4,5,5-tetramethyl-1,3,2-dioxaborolan-2-yl)isoquinolin-1-amine (9h) (53.8 mg, 0.199 mmol), K₃PO₄ (2M aqueous solution, 0.149 mL, 0.597 mmol), PCy₃ (22.33 mg, 0.08 mmol) and Pd₂(dba)₃ (36.5 mg, 0.040 mmol) to afford after workup and purification using method-U, ethyl 2-(2-((5-(1-aminoisoquinolin-7-yl)-2-(1-(2-methoxyethyl)azetidin-3-yl)-2H-indazol-3-yl)methoxy)phenyl)acetate (122a) (54 mg, 48.0% yield) as a clear oil; MS (ES+): 566.3 (M+1).

### Step-2: Preparation of 2-(2-((5-(1-aminoisoquinolin-7-yl)-2-(1-(2-methoxyethyl)azetidin-3-yl)-2H-indazol-3-yl)methoxy)phenyl)acetic acid (122b)

Compound 122b was prepared according to the procedure reported step-2 of scheme-1 from ethyl 2-(2-((5-(1-aminoisoquinolin-7-yl)-2-(1-(2-methoxyethyl)azetidin-3-yl)-2H-indazol-3-yl)methoxy)phenyl)acetate (122a) (54 mg, 0.095 mmol) in THF (3 mL) using a solution of LiOH·H₂O (14.3 mg, 0.597 mmol) in water (1 mL) to afford after workup and purification using method-M, 2-(2-((5-(1-aminoisoquinolin-7-yl)-2-(1-(2-methoxyethyl)azetidin-3-yl)-2H-indazol-3-yl)methoxy)phenyl)acetic acid (122b) (27 mg, 25.2% yield) HCl salt as a white solid; ¹H NMR (300 MHz, DMSO-d₆) δ 13.56 (s, 1H, D₂O exchangeable), 12.21 (s, 1H, D₂O exchangeable), 11.05 (m, 1H, D₂O exchangeable), 9.38 (s, 1H, D₂O exchangeable), 9.12 (d, J=7.9 Hz, 1H), 8.62 (d, J=7.3 Hz, 1H), 8.45 (d, J=8.5 Hz, 1H), 8.04 (t, J=8.7 Hz, 2H), 7.91 (d, J=9.1 Hz, 1H), 7.70 (d, J=7.0 Hz, 1H), 7.34 (d, J=3.9 Hz, 2H), 7.30-7.18 (m, 2H), 7.04-6.91 (m, 1H), 5.97-5.63 (m, 3H), 4.89-4.54 (m, 4H), 3.67 (s, 3H), 3.62-3.57 (m, 1H), 3.51 (s, 2H), 3.33-3.29 (m, 3H); MS (ES+) 538.2 (M+1); (ES−) 536.2 (M−1).

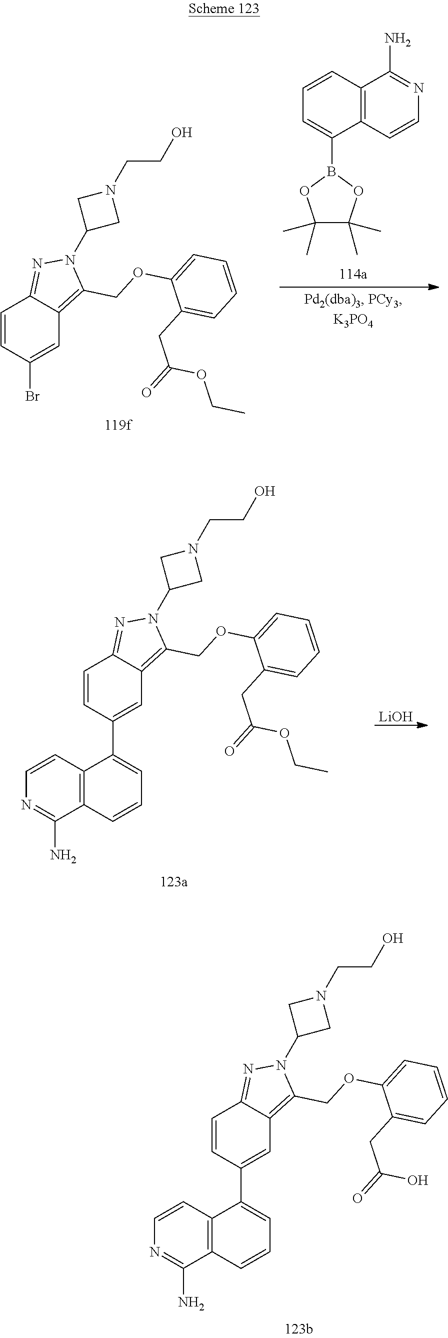 [SMILES: CCOC(=O)Cc1ccccc1OCc1c2cc(Br)ccc2nn1C1CN(CCO)C1 ; CC1(C)OB(c2cccc3c(N)nccc23)OC1(C)C ; CCOC(=O)Cc1ccccc1OCc1c2cc(-c3cccc4c(N)nccc34)ccc2nn1C1CN(CCO)C1 ; "LiOH" ; Nc1nccc2c(-c3ccc4nn(C5CN(CCO)C5)c(COc5ccccc5CC(=O)O)c4c3)cccc12]

### Preparation of 2-(2-((5-(1-aminoisoquinolin-5-yl)-2-(1-(2-hydroxyethyl)azetidin-3-yl)-2H-indazol-3-yl)methoxy)phenyl)acetic acid (123b)

### Step-1: Preparation of ethyl 2-(2-((5-(1-aminoisoquinolin-5-yl)-2-(1-(2-hydroxyethyl)azetidin-3-yl)-2H-indazol-3-yl)methoxy)phenyl)acetate (123a)

Compound 123a was prepared according to the procedure reported in step-3 of scheme-112 from ethyl 2-(2-((5-bromo-2-(1-(2-hydroxyethyl)azetidin-3-yl)-2H-indazol-3-yl)methoxy)phenyl)acetate (119f) (90 mg, 0.184 mmol) in dioxane/2-MeTHF (4 mL, 3:1) using 5-(4,4,5,5-tetramethyl-1,3,2-dioxaborolan-2-yl)isoquinolin-1-amine (114a) (74.7 mg, 0.276 mmol), K₃PO₄ (2M aqueous solution, 0.138 mL, 0.553 mmol), PCy₃ (20.67 mg, 0.074 mmol) and Pd₂(dba)₃ (33.8 mg, 0.037 mmol) to afford after workup and purification using method-U, ethyl 2-(2-((5-(1-aminoisoquinolin-5-yl)-2-(1-(2-hydroxyethyl)azetidin-3-yl)-2H-indazol-3-yl)methoxy)phenyl)acetate (123a) (53 mg, 052.1% yield) as a clear oil; MS (ES+): 552.3 (M+1).

### Step-2: Preparation of 2-(2-((5-(1-aminoisoquinolin-5-yl)-2-(1-(2-hydroxyethyl)azetidin-3-yl)-2H-indazol-3-yl)methoxy)phenyl)acetic acid (123b)

Compound 123b was prepared according to the procedure reported step-2 of scheme-1 from ethyl 2-(2-((5-(1-aminoisoquinolin-5-yl)-2-(1-(2-hydroxyethyl)azetidin-3-yl)-2H-indazol-3-yl)methoxy)phenyl)acetate (123a) (53 mg, 0.096 mmol) in THF (3 mL) using a solution of LiOH·H₂O (13.24 mg, 0.553 mmol) in water (1 mL) to afford after workup and purification using method-M, 2-(2-((5-(1-aminoisoquinolin-S-yl)-2-(1-(2-hydroxyethyl)azetidin-3-yl)-2H-indazol-3-yl)methoxy)phenyl)acetic acid (123b) (20 mg, 20.73% yield) HCl salt as a white solid; ¹H NMR (300 MHz, DMSO-d₆) δ 13.50 (s, 1H, D₂O exchangeable), 12.14 (s, 1H, DO exchangeable), 9.29 (s, 2H, D₂O exchangeable), 8.67 (d, J=8.3 Hz, 1H), 8.06 (s, 1H), 7.97 (d, J=7.2 Hz, 1H), 7.93-7.83 (m, 2H), 7.67 (d, J=7.3 Hz, 1H), 7.42 (d, J=9.0 Hz, 1H), 7.33-7.18 (m, 3H), 7.01-6.88 (m, 2H), 5.97-5.74 (m, 1H), 5.66 (s, 2H), 5.32 (s, 1H, D₂O) exchangeable), 4.89-4.58 (m, 4H), 3.75 (t, J=5.2 Hz, 2H), 3.59-3.46 (m, 4H); MS (ES+) 524.3 (M+1), (ES−) 522.2 (M−1).

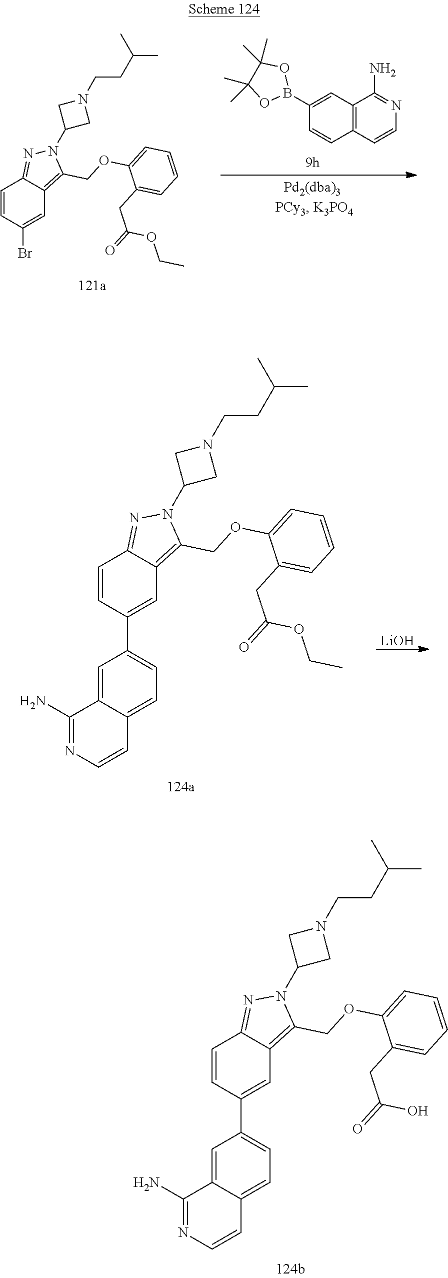

### Preparation of 2-(2-((5-(1-aminoisoquinolin-7-yl)-2-(1-isopentylazetidin-3-yl)-2H-indazol-3-yl)methoxy)phenyl)acetic acid (124b)

### Step-1: Preparation of ethyl 2-(2-((5-(1-aminoisoquinolin-7-yl)-2-(1-isopentylazetidin-3-yl)-2H-indazol-3-yl)methoxy)phenyl)acetate (124a)

Compound 124a was prepared according to the procedure reported in step-3 of scheme-112 from ethyl 2-(2-((5-bromo-2-(1-isopentylazetidin-3-yl)-2H-indazol-3-yl)methoxy)phenyl)acetate (121a) (135 mg, 0.262 mmol) in dioxane/2-MeTHF (4 mL, 3:1) using 7-(4,4,5,5-tetramethyl-1,3,2-dioxaborolan-2-yl)isoquinolin-1-amine (9h) (106 mg, 0.394 mmol), K₃PO₄ (2M aqueous solution, 0.197 mL, 0.787 mmol), PCy₃ (29.4 mg, 0.105 mmol) and Pd₂(dba)₃ (48.1 mg, 0.052 mmol) to afford after workup and purification using method-U, ethyl 2-(2-((5-(1-aminoisoquinolin-7-yl)-2-(1-isopentylazetidin-3-yl)-2H-indazol-3-yl)methoxy)phenyl)acetate (124a) (63 mg, 41.6% yield) as a clear oil; MS (ES+): 578.3 (M+1).

### Step-2: Preparation of 2-(2-((5-(1-aminoisoquinolin-7-yl)-2-(1-isopentylazetidin-3-yl)-2H-indazol-3-yl)methoxy)phenyl)acetic acid (124b)

Compound 124b was prepared according to the procedure reported step-2 of scheme-1 from ethyl 2-(2-((5-(1-aminoisoquinolin-7-yl)-2-(1-isopentylazetidin-3-yl)-2H-indazol-3-yl)methoxy)phenyl)acetate (124a) (63 mg, 0.109 mmol) in THF (3 mL) using a solution of LiOH·H₂O (18.85 mg, 0.787 mmol) in water (1 mL) to afford after workup and purification using method-M, 2-(2-((5-(1-aminoisoquinolin-7-yl)-2-(1-isopentylazetidin-3-yl)-2H-indazol-3-yl)methoxy)phenyl)acetic acid (124b) (38 mg, 26.3% yield) HCl salt as a white solid; 1H NMR (300 MHz, DMSO-d₆) δ 13.42 (s, 1H, D₂O) exchangeable), 12.13 (s, 1H, D₂O exchangeable), 11.00 (d, J=116.5 Hz, 1H, D₂O exchangeable), 9.28 (s, 2H, D₂O exchangeable), 9.07 (s, 1H), 8.59 (s, 1H), 8.45 (d, J=8.5, 1.6 Hz, 1H), 8.09-7.96 (m, 2H), 7.91 (d, J=9.1 Hz, 1H), 7.70 (d, J=6.9 Hz, 1H), 7.34 (d, J=4.2 Hz, 2H), 7.26 (dd, J=7.2, 3.7 Hz, 2H), 7.05-6.91 (m, 1H), 5.93-5.75 (m, 1H), 5.71 (s, 2H), 4.90-4.41 (m, 4H), 3.51 (s, 2H), 1.77-1.57 (m, 1H), 1.57-1.40 (m, 2H), 0.93 (d, J=6.5 Hz, 6H); MS (ES+): 550.3 (M+1), (ES−): 548.3 (M−1).

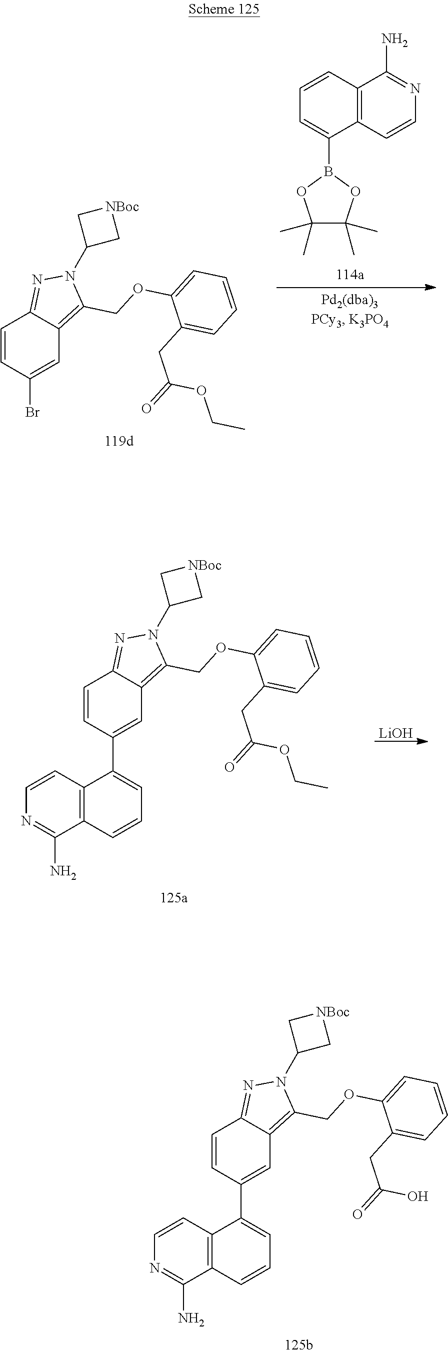 [SMILES: CCOC(=O)Cc1ccccc1OCc1c2cc(Br)ccc2nn1C1CN(C(=O)OC(C)(C)C)C1 ; CC1(C)OB(c2cccc3c(N)nccc23)OC1(C)C ; CCOC(=O)Cc1ccccc1OCc1c2cc(-c3cccc4c(N)nccc34)ccc2nn1C1CN(C(=O)OC(C)(C)C)C1 ; "LiOH" ; CC(C)(C)OC(=O)N1CC(n2nc3ccc(-c4cccc5c(N)nccc45)cc3c2COc2ccccc2CC(=O)O)C1]

### Preparation of 2-(2-((5-(1-aminoisoquinolin-5-yl)-2-(1-(tert-butoxycarbonyl)azetidin-3-yl)-2H-indazol-3-yl)methoxy)phenyl)acetic acid (125b)

### Step-1: Preparation of tert-butyl 3-(5-(1-aminoisoquinolin-5-yl)-3-((2-(2-ethoxy-2-oxoethyl)phenoxy)methyl)-2H-indazol-2-yl)azetidine-1-carboxylate (125a)

Compound 125a was prepared according to the procedure reported in step-3 of scheme-112 from tert-butyl 3-(5-bromo-3-((2-(2-ethoxy-2-oxoethyl)phenoxy)methyl)-2H-indazol-2-yl)azetidine-1-carboxylate (119d) (340 mg, 0.624 mmol) in dioxane (4 mL, 3:1) was added 5-(4,4,5,5-tetramethyl-1,3,2-dioxaborolan-2-yl)isoquinolin-1-amine (114a) (253 mg, 0.937 mmol), K₃PO₄ (2M aqueous solution, 0.468 mL, 1.873 mmol), PCy₃ (70.1 mg, 0.250 mmol) and Pd₂(dba)₃ (114 mg, 0.125 mmol) to afford after workup and purification using method-U, tert-butyl 3-(5-(1-aminoisoquinolin-5-yl)-3-((2-(2-ethoxy-2-oxoethyl)phenoxy)methyl)-2H-indazol-2-yl)azetidine-1-carboxylate (125a) (182 mg, 48.0% yield) as a clear oil; MS (ES+): 608.3 (M+1).

### Step-2: Preparation of 2-(2-((5-(1-aminoisoquinolin-5-yl)-2-(1-(tert-butoxycarbonyl)azetidin-3-yl)-2H-indazol-3-yl)methoxy)phenyl)acetic acid (125b)

Compound 125b was prepared according to the procedure reported step-2 of scheme-1 from tert-butyl 3-(5-(1-aminoisoquinolin-5-yl)-3-((2-(2-ethoxy-2-oxoethyl)phenoxy)methyl)-2H-indazol-2-yl)azetidine-1-carboxylate (125a) (182 mg, 0.299 mmol) in THF (3 mL) using a solution of LiOH·H₂O (44.9 mg, 1.873 mmol) in water (1 mL) to afford after workup and purification using method-M, 2-(2-((5-(1-aminoisoquinolin-5-yl)-2-(1-(tert-butoxycarbonyl)azetidin-3-yl)-2H-indazol-3-yl)methoxy)phenyl)acetic acid (125b) (126 mg, 34.8% yield) HCl salt as a white solid; 1H NMR (300 MHz, DMSO-d₆) δ 13.53 (s, 1H, D₂O exchangeable), 12.17 (s, 1H, D₂O exchangeable), 9.29 (s, 2H, D₂O exchangeable), 8.67 (d, J=8.3 Hz, 1H), 8.01 (s, 1H), 7.96 (d, J=7.2 Hz, 1H), 7.92-7.80 (m, 2H), 7.66 (d, J=7.2 Hz, 1H), 7.37 (dd, J=8.9, 1.6 Hz, 1H), 7.30-7.23 (m, 2H), 7.21 (d, J=7.4 Hz, 1H), 7.02-6.86 (m, 2H), 5.81-5.48 (m, 3H), 4.63-4.17 (m, 4H), 3.47 (s, 2H), 1.44 (s, 9H); MS (ES+) 580.2 (M+1); (ES−) 578.2 (M−1).

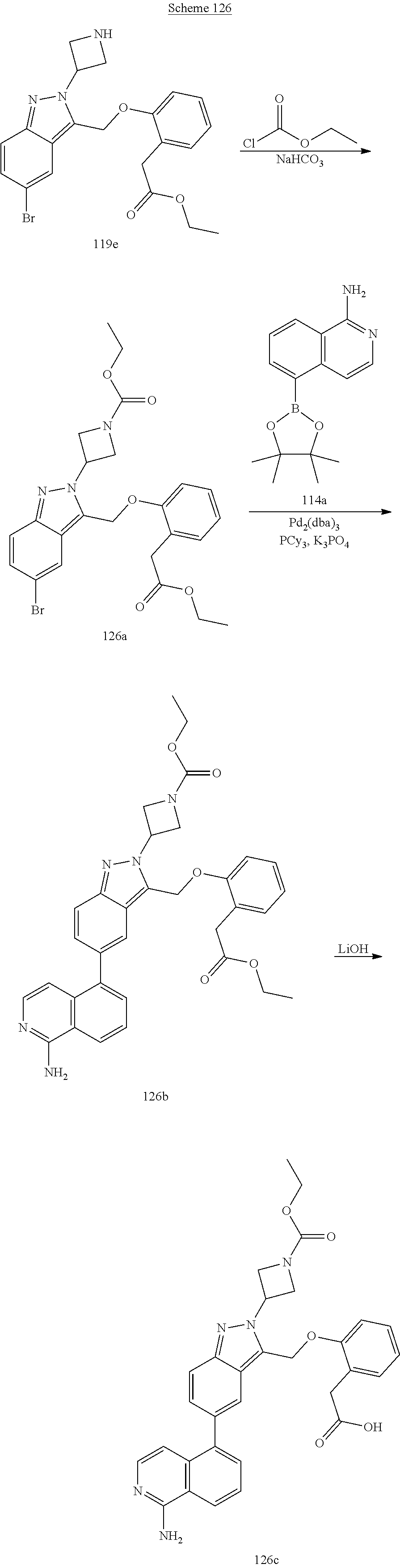

### Preparation of 2-(2-((5-(1-aminoisoquinolin-5-yl)-2-(1-(ethoxycarbonyl)azetidin-3-yl)-2H-indazol-3-yl)methoxy)phenyl)acetic acid (126c)

### Step-1: Preparation of ethyl 3-(5-bromo-3-((2-(2-ethoxy-2-oxoethyl)phenoxy)methyl)-2H-indazol-2-yl)azetidine-1-carboxylate (126a)

To a solution of ethyl 2-(2-((2-(azetidin-3-yl)-5-bromo-2H-indazol-3-yl)methoxy)phenyl)acetate (119e) (450 mg, 1.013 mmol) in THF (3 mL) and water (3 mL) was added sodium bicarbonate (128 mg, 1.519 mmol) and ethyl chloroformate (0.145 mL, 1.519 mmol) at 0° ° C. and stirred at RT overnight. The reaction mixture was diluted with EtOAc (100 mL), washed with water and brine. The organic layer was dried, filtered and concentrated. The residue obtained was purified using method-J to give ethyl 3-(5-bromo-3-((2-(2-ethoxy-2-oxoethyl)phenoxy)methyl)-2H-indazol-2-yl)azetidine-1-carboxylate (126a) (290 mg, 55.5% yield) as a clear oil; 1H NMR (300 MHz, DMSO-d₆) δ 8.18 (d, J=1.9 Hz, 1H), 7.72 (d, J=9.1 Hz, 1H), 7.46-7.38 (m, 1H), 7.36-7.30 (m, 1H), 7.29-7.19 (m, 2H), 7.03-6.89 (m, 1H), 5.76-5.63 (m, 1H), 5.57 (s, 2H), 4.54-4.29 (m, 4H), 4.10-4.04 (m, 2H), 3.78 (q. J=7.1 Hz, 2H), 3.52 (s, 2H), 1.20-1.16 (m, 3H), 0.88 (t, J=7.7, 6.5 Hz, 3H); MS (ES+): 516.1 and 518.1 (M+1); (ES−): 514.0 and 516.1 (M−1).

### Step-2: Preparation of ethyl 3-(5-(1-aminoisoquinolin-5-yl)-3-((2-(2-ethoxy-2-oxoethyl)phenoxy)methyl)-2H-indazol-2-yl)azetidine-1-carboxylate (126b)

Compound 126b was prepared according to the procedure reported in step-3 of scheme-112 from ethyl 3-(5-bromo-3-((2-(2-ethoxy-2-oxoethyl)phenoxy)methyl)-2H-indazol-2-yl)azetidine-1-carboxylate (126a) (145 mg, 0.281 mmol) in dioxane/2-MeTHF (4 mL, 3:1) using 5-(4,4,5,5-tetramethyl-1.3,2-dioxaborolan-2-yl)isoquinolin-1-amine (114a) (114 mg, 0.421 mmol), K₃PO₄ (2M aqueous solution, 0.211 mL, 0.842 mmol), PCy₃ (23.62 mg, 0.084 mmol) and Pd₂(dba)₃ (51.4 mg, 0.056 mmol) to afford after workup and purification using method-U, ethyl 3-(5-(1-aminoisoquinolin-5-yl)-3-((2-(2-ethoxy-2-oxoethyl)phenoxy)methyl)-2H-indazol-2-yl)azetidine-1-carboxylate (126b) (70 mg, 43.0% yield) as a clear oil; MS (ES+): 580.3 (M+1).

### Step-3: Preparation of 2-(2-((5-(1-aminoisoquinolin-5-yl)-2-(1-(ethoxycarbonyl)azetidin-3-yl)-2H-indazol-3-yl)methoxy)phenyl)acetic acid (126c)

Compound 126c was prepared according to the procedure reported step-2 of scheme-1 from ethyl 3-(5-(1-aminoisoquinolin-5-yl)-3-((2-(2-ethoxy-2-oxoethyl)phenoxy)methyl)-2H-indazol-2-yl)azetidine-1-carboxylate (126b) (70 mg, 0.121 mmol) in THF (3 mL) using a solution of LiOH·H₂O) (20.17 mg, 0.842 mmol) in water (1 mL) to afford after workup and purification using method-M, 2-(2-((5-(1-aminoisoquinolin-5-yl)-2-(1-(ethoxycarbonyl)azetidin-3-yl)-2H-indazol-3-yl)methoxy)phenyl)acetic acid (126e) (32 mg, 20.66% yield) as a white solid; ¹H NMR (300 MHz, DMSO-d₆) δ 13.42 (s, 1H, D₂O exchangeable), 12.16 (s, 1H, DO exchangeable), 9.27 (s, 2H, D₂O exchangeable), 8.66 (d, J=8.4 Hz, 1H), 8.02 (s, 1H), 7.97 (d, J=7.3 Hz, 1H), 7.94-7.83 (m, 2H), 7.66 (d, J=7.2 Hz, 1H), 7.43-7.34 (m, 1H), 7.34-7.26 (m, 2H), 7.22 (d, J=7.4 Hz, 1H), 7.05-6.86 (m, 2H), 5.79-5.67 (m, 1H), 5.64 (s, 2H), 4.64-4.31 (m, 4H), 4.10 (q, J=7.1 Hz, 2H), 3.48 (s, 2H), 1.23 (t, J=7.1 Hz, 3H); MS (ES+): 552.2 (M+1); (ES−): 550.1 (M−1).

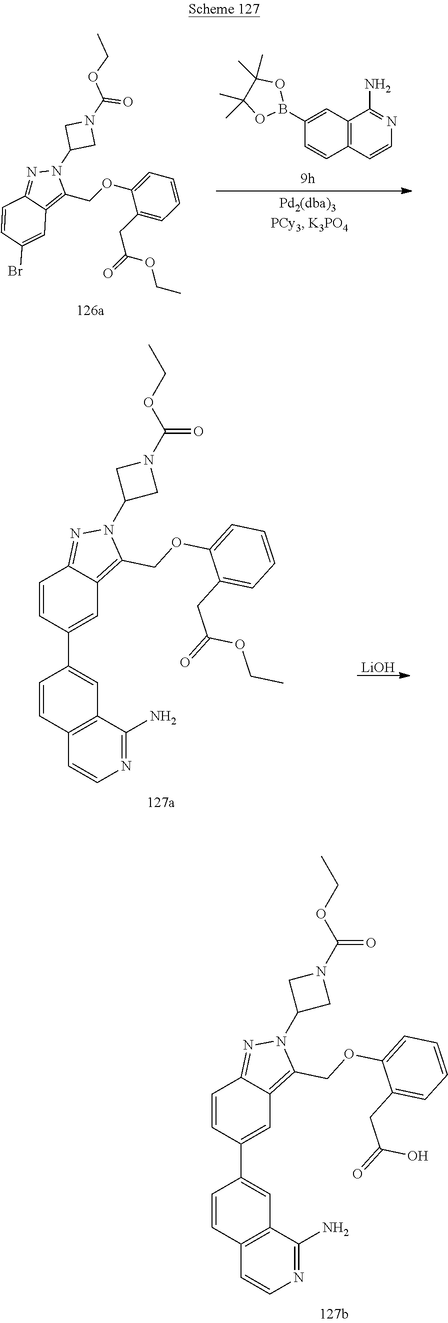 [SMILES: CCOC(=O)Cc1ccccc1OCc1c2cc(Br)ccc2nn1C1CN(C(=O)OCC)C1 ; CC1(C)OB(c2ccc3ccnc(N)c3c2)OC1(C)C ; CCOC(=O)Cc1ccccc1OCc1c2cc(-c3ccc4ccnc(N)c4c3)ccc2nn1C1CN(C(=O)OCC)C1 ; "LiOH" ; CCOC(=O)N1CC(n2nc3ccc(-c4ccc5ccnc(N)c5c4)cc3c2COc2ccccc2CC(=O)O)C1]

### Preparation of 2-(2-((5-(1-aminoisoquinolin-7-yl)-2-(1-(ethoxycarbonyl)azetidin-3-yl)-2H-indazol-3-yl)methoxy)phenyl)acetic acid (127b)

### Step-1: Preparation of ethyl 3-(5-(1-aminoisoquinolin-7-yl)-3-((2-(2-ethoxy-2-oxoethyl)phenoxy)methyl)-2H-indazol-2-yl)azetidine-1-carboxylate (127a)

Compound 127a was prepared according to the procedure reported in step-3 of scheme-112 from ethyl 3-(5-bromo-3-((2-(2-ethoxy-2-oxoethyl)phenoxy)methyl)-2H-indazol-2-yl)azetidine-1-carboxylate (126a) (145 mg, 0.281 mmol) in dioxane/2-MeTHF (4 mL, 3:1) using 7-(4,4,5,5-tetramethyl-1,3,2-dioxaborolan-2-yl)isoquinolin-1-amine (9h) (76 mg, 0.281 mmol), K₃PO₄ (2M aqueous solution, 0.211 mL, 0.842 mmol). PCy₃ (23.62 mg, 0.084 mmol) and Pd₂(dba)₃ (51.4 mg, 0.056 mmol) to afford after workup and purification using method-U, ethyl 3-(5-(1-aminoisoquinolin-7-yl)-3-((2-(2-ethoxy-2-oxoethyl)phenoxy)methyl)-2H-indazol-2-yl)azetidine-1-carboxylate (127a) (69 mg, 42.4% yield) as a clear oil; MS (ES+): 580.3 (M+1).

### Step-2: Preparation of 2-(2-((5-(1-aminoisoquinolin-7-yl)-2-(1-(ethoxycarbonyl)azetidin-3-yl)-2H-indazol-3-yl)methoxy)phenyl)acetic acid (127b)

Compound 127b was prepared according to the procedure reported step-2 of scheme-1 from ethyl 3-(5-(1-aminoisoquinolin-7-yl)-3-((2-(2-ethoxy-2-oxoethyl)phenoxy)methyl)-2H-indazol-2-yl)azetidine-1-carboxylate (127a) (69 mg, 0.119 mmol) in THF (3 mL) using a solution of LiOH·H₂O (20.17 mg, 0.842 mmol) in water (1 mL) to afford after workup and purification using method-M, 2-(2-((5-(1-aminoisoquinolin-7-yl)-2-(1-(ethoxycarbonyl)azetidin-3-yl)-2H-indazol-3-yl)methoxy)phenyl)acetic acid (127b) (24 mg, 15.50% yield) HCl salt as a white solid; ¹H NMR (300 MHz, DMSO-d₆) δ 13.34 (s, 1H), 12.15 (s, 1H), 9.24 (s, 1H), 9.02 (s, 1H), 8.52 (s, 1H), 8.50-8.42 (m, 1H), 8.06 (d, J=8.5 Hz, 1H), 8.01-7.88 (m, 2H), 7.69 (d, J=6.9 Hz, 1H), 7.33 (d, J=4.2 Hz, 2H), 7.31-7.20 (m, 2H), 7.03-6.92 (m, 1H), 5.81-5.61 (m, 3H), 4.62-4.46 (m, 2H), 4.46-4.31 (m, 2H), 4.10 (q, J=7.1 Hz, 2H), 3.50 (s, 2H), 1.22 (t, J=7.1 Hz, 3H); MS (ES+) 552.2 (M+1); (ES−) 550.1 (M−1).

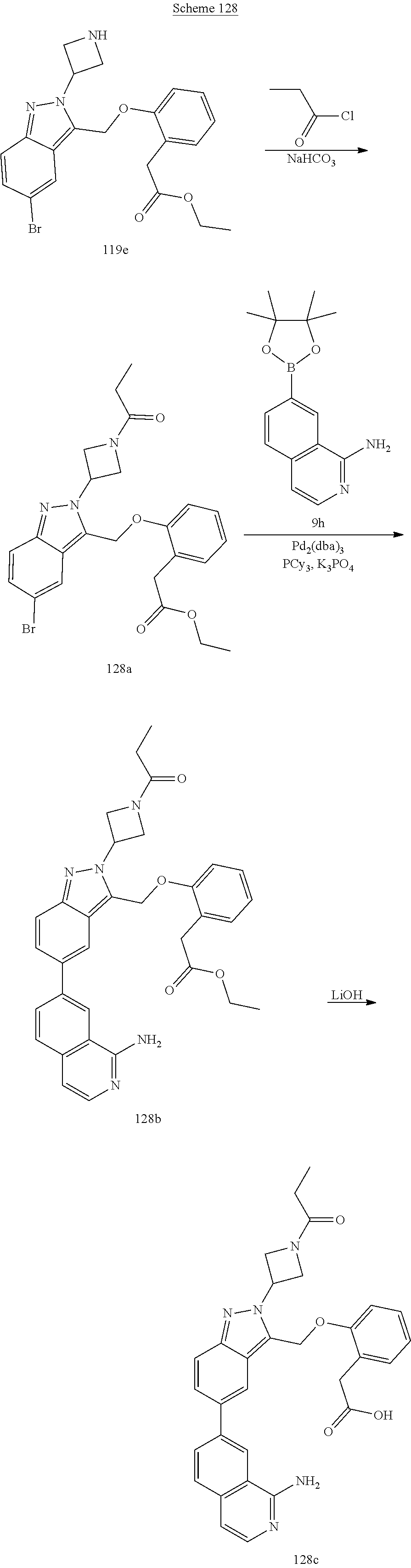

### Preparation of 2-(2-((5-(1-aminoisoquinolin-7-yl)-2-(1-propionylazetidin-3-yl)-2H-indazol-3-yl)methoxy)phenyl)acetic acid (128c)

### Step-1: Preparation of ethyl 2-(2-((5-bromo-2-(1-propionylazetidin-3-yl)-2H-indazol-3-yl)methoxy)phenyl)acetate (128a)

Compound 128a was prepared according to the procedure reported in step-1 of scheme-126 from ethyl 2-(2-((2-(azetidin-3-yl)-5-bromo-2H-indazol-3-yl)methoxy)phenyl)acetate (119e) (450 mg, 1.013 mmol) in THF (3 mL) and water (3 mL) using NaHCO₃ (128 mg, 1.519 mmol) and propionyl chloride (141 mg, 1.519 mmol) to afford after workup and purification using method-J, ethyl 2-(2-((5-bromo-2-(1-propionylazetidin-3-yl)-2H-indazol-3-yl)methoxy)phenyl)acetate (128a) (285 mg, 56.2% yield) as a clear oil; MS (ES+): 500.1 and 502.1 (M+1); (ES−): 498.0 and 500.0 (M−1).

### Step-2: Preparation of ethyl 2-(2-((5-(1-aminoisoquinolin-7-yl)-2-(1-propionylazetidin-3-yl)-2H-indazol-3-yl)methoxy)phenyl)acetate (128b)

Compound 128b was prepared according to the procedure reported in step-3 of scheme-112 from ethyl 2-(2-((5-bromo-2-(1-propionylazetidin-3-yl)-2H-indazol-3-yl)methoxy)phenyl)acetate (128a) (145 mg, 0.29 mmol) in dioxane/2-MeTHF (4 mL, 3:1) using 7-(4,4,5,5-tetramethyl-1,3,2-dioxaborolan-2-yl)isoquinolin-1-amine (9h) (78 mg, 0.29 mmol), K₃PO₄ (2M aqueous solution, 0.217 mL, 0.869 mmol), PCy₃ (24.38 mg, 0.087 mmol) and Pd₂(dba)₃ (53.1 mg, 0.058 mmol) to afford after workup and purification using method-U, ethyl 2-(2-((5-(1-aminoisoquinolin-7-yl)-2-(1-propionylazetidin-3-yl)-2H-indazol-3-yl)methoxy)phenyl)acetate (128b) (68 mg, 41.6% yield) as a clear oil; MS (ES+): 564.3 (M+1).

### Step-3: Preparation of 2-(2-((5-(1-aminoisoquinolin-7-yl)-2-(1-propionylazetidin-3-yl)-2H-indazol-3-yl)methoxy)phenyl)acetic acid (128c)

Compound 128e was prepared according to the procedure reported step-2 of scheme-1 from ethyl 2-(2-((5-(1-aminoisoquinolin-7-yl)-2-(1-propionylazetidin-3-yl)-2H-indazol-3-yl)methoxy)phenyl)acetate (128b) (68 mg, 0.121 mmol) in THF (3 mL) using a solution of LiOH·H₂O (20.82 mg, 0.869 mmol) in water (1 mL) to afford after workup and purification using method-M, 2-(2-((5-(1-aminoisoquinolin-7-yl)-2-(1-propionylazetidin-3-yl)-2H-indazol-3-yl)methoxy)phenyl)acetic acid (128c) (33 mg, 21.26% yield) HCl salt as a white solid; 1H NMR (300 MHz, DMSO-d₆) δ 13.23 (s, 1H), 12.19 (s, 1H), 9.19 (s, 1H), 9.00 (s, 1H), 8.51 (s, 1H), 8.47 (d, J=8.4 Hz, 1H), 8.07 (d, J=8.5 Hz, 1H), 8.02-7.88 (m, 2H), 7.69 (d, J=6.9 Hz, 1H), 7.38-7.31 (m, 2H), 7.31-7.21 (m, 2H), 7.05-6.93 (m, 1H), 5.83-5.54 (m, 3H), 4.70 (t, J=8.4 Hz, 1H), 4.65-4.54 (m, 1H), 4.45 (t, J=8.9 Hz, 1H), 4.40-4.29 (m, 1H), 3.50 (s, 2H), 2.17 (q, J=7.5 Hz, 2H), 1.03 (t, J=7.4 Hz, 3H); MS (ES+): 536.3 (M+1); (ES−): 534.1 (M−1).

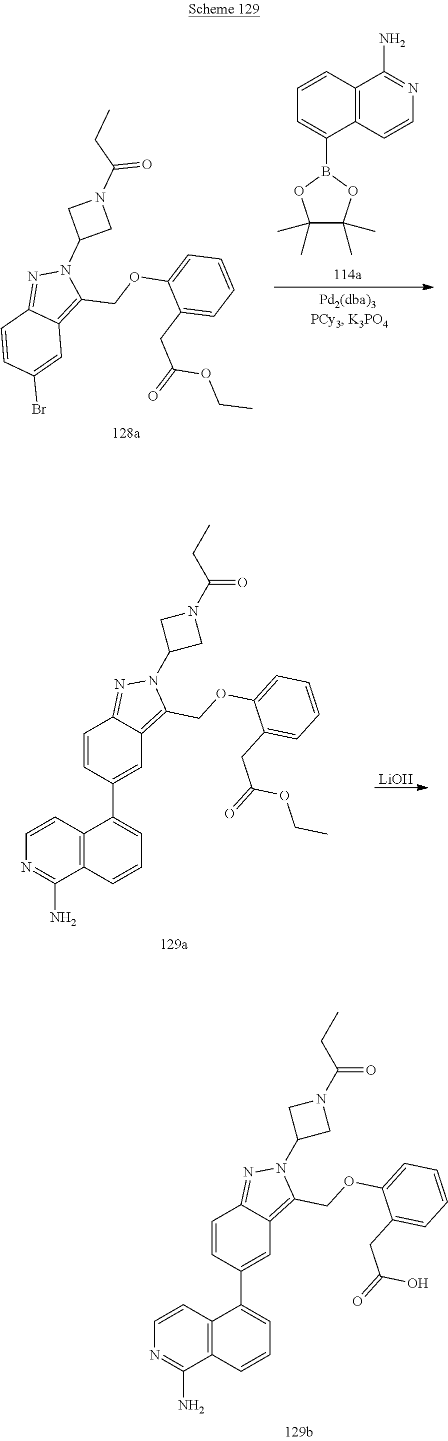 [SMILES: CCOC(=O)Cc1ccccc1OCc1c2cc(Br)ccc2nn1C1CN(C(=O)CC)C1 ; CC1(C)OB(c2cccc3c(N)nccc23)OC1(C)C ; CCOC(=O)Cc1ccccc1OCc1c2cc(-c3cccc4c(N)nccc34)ccc2nn1C1CN(C(=O)CC)C1 ; "LiOH" ; CCC(=O)N1CC(n2nc3ccc(-c4cccc5c(N)nccc45)cc3c2COc2ccccc2CC(=O)O)C1]

### Preparation of 2-(2-((5-(1-aminoisoquinolin-5-yl)-2-(1-propionylazetidin-3-yl)-2H-indazol-3-yl)methoxy)phenyl)acetic acid (129b)

### Step-1: Preparation of ethyl 2-(2-((5-(1-aminoisoquinolin-5-yl)-2-(1-propionylazetidin-3-yl)-2H-indazol-3-yl)methoxy)phenyl)acetate (129a)

Compound 129a was prepared according to the procedure reported in step-3 of scheme-112 from ethyl 2-(2-((5-bromo-2-(1-propionylazetidin-3-yl)-2H-indazol-3-yl)methoxy)phenyl)acetate (128a) (145 mg, 0.290 mmol) in dioxane/2-MeTHF (4 mL, 3:1) using 5-(4,4,5,5-tetramethyl-1,3,2-dioxaborolan-2-yl)isoquinolin-1-amine (114a) (117 mg, 0.435 mmol), K₃PO₄ (2M aqueous solution, 0.217 mL, 0.869 mmol), PCy₃ (24.38 mg, 0.087 mmol) and Pd₂(dba)₃ (53.1 mg, 0.058 mmol) to afford after workup and purification using method-U, ethyl 2-(2-((5-(1-aminoisoquinolin-5-yl)-2-(1-propionylazetidin-3-yl)-2H-indazol-3-yl)methoxy)phenyl)acetate (129a) (66 mg, 40.4% yield) as a clear oil; MS (ES+): 564.3 (M+1).

### Step-2: Preparation of 2-(2-((5-(1-aminoisoquinolin-5-yl)-2-(1-propionylazetidin-3-yl)-2H-indazol-3-yl)methoxy)phenyl)acetic acid (129b)

Compound 129b was prepared according to the procedure reported step-2 of scheme-1 from ethyl 2-(2-((5-(1-aminoisoquinolin-5-yl)-2-(1-propionylazetidin-3-yl)-2H-indazol-3-yl)methoxy)phenyl)acetate (129a) (66 mg, 0.117 mmol) in THF (3 mL) using a solution of LiOH·H₂O (20.82 mg, 0.869 mmol) in water (1 mL) to afford after workup and purification using method-M, 2-(2-((5-(1-aminoisoquinolin-5-yl)-2-(1-propionylazetidin-3-yl)-2H-indazol-3-yl)methoxy)phenyl)acetic acid (129b) (7 mg, 4.51% yield) HCl salt as a white solid; 1H NMR (300 MHz, DMSO-d₆) δ 13.15 (s, 1H, D₂O exchangeable), 12.17 (s, 1H, D₂O exchangeable), 9.09 (s, 2H, D₂O exchangeable), 8.62 (d, J=8.3 Hz, 1H), 8.03 (s, 1H), 7.97 (d, J=7.3 Hz, 1H), 7.93-7.81 (m, 2H), 7.65 (d, J=7.2 Hz, 1H), 7.42-7.34 (m, 1H), 7.33-7.25 (m, 2H), 7.22 (d, J=7.4 Hz, 1H), 7.02-6.89 (m, 2H), 5.80-5.69 (m, 1H), 5.65 (s, 2H), 4.71 (t, J=8.4 Hz, 1H), 4.66-4.55 (m, 1H), 4.46 (t, J=9.0 Hz, 1H), 4.42-4.31 (m, 1H), 3.49 (s, 2H), 2.18 (q, J=7.5 Hz, 2H), 1.03 (t, J=7.5 Hz, 3H); MS (ES+) 536.2 (M+1); (ES−) 534.2 (M−1).

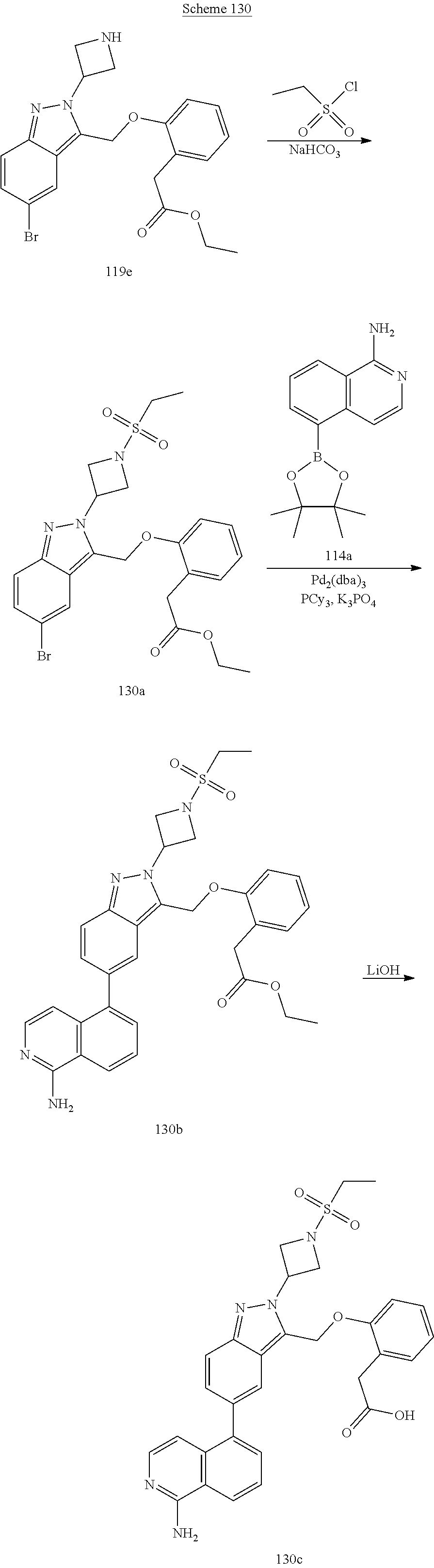

### Preparation of 2-(2-((5-(1-aminoisoquinolin-5-yl)-2-(1-(ethylsulfonyl)azetidin-3-yl)-2H-indazol-3-yl)methoxy)phenyl)acetic acid (130c)

### Step-1: Preparation of ethyl 2-(2-((5-bromo-2-(1-(ethylsulfonyl)azetidin-3-yl)-2H-indazol-3-yl)methoxy)phenyl)acetate (130a)

Compound 130a was prepared according to the procedure reported in step-1 of scheme-126 from ethyl 2-(2-((2-(azetidin-3-yl)-5-bromo-2H-indazol-3-yl)methoxy)phenyl)acetate (119e) (450 mg, 1.013 mmol) in THF (3 mL) and water (3 mL) using NaHCO₃ (128 mg, 1.519 mmol) and ethanesulfonyl chloride (195 mg, 1.519 mmol) to afford after workup and purification using method-J, ethyl 2-(2-((5-bromo-2-(1-(ethylsulfonyl)azetidin-3-yl)-2H-indazol-3-yl)methoxy)phenyl)acetate (130a) (320 mg, 58.9% yield) as a clear oil; MS (ES+): 536.1 and 538.1 (M+1); (ES−): 534.0 and 536.0 (M−1).

### Step-2: Preparation of ethyl 2-(2-((5-(1-aminoisoquinolin-5-yl)-2-(1-(ethylsulfonyl)azetidin-3-yl)-2H-indazol-3-yl)methoxy)phenyl)acetate (130b)

Compound 130b was prepared according to the procedure reported in step-3 of scheme-112 from ethyl 2-(2-((5-bromo-2-(1-(ethylsulfonyl)azetidin-3-yl)-2H-indazol-3-yl)methoxy)phenyl)acetate (130a) (150 mg, 0.280 mmol) in dioxane/2-MeTHF (4 mL, 3:1) using 5-(4,4,5,5-tetramethyl-1,3,2-dioxaborolan-2-yl)isoquinolin-1-amine (114a) (113 mg, 0.419 mmol), K₃PO₄ (2M aqueous solution, 0.21 mL, 0.839 mmol), PCy₃ (23.52 mg, 0.084 mmol) and Pd₂(dba)₃ (51.2 mg, 0.056 mmol) to afford after workup and purification using method-U, ethyl 2-(2-((5-(1-aminoisoquinolin-5-yl)-2-(1-(ethylsulfonyl)azetidin-3-yl)-2H-indazol-3-yl)methoxy)phenyl)acetate (130b) (76 mg, 45.3% yield) as a clear oil; MS (ES+): 600.3 (M+1); (ES−): 598.2 (M−1).

### Step-3: Preparation of 2-(2-((5-(1-aminoisoquinolin-5-yl)-2-(1-(ethylsulfonyl)azetidin-3-yl)-2H-indazol-3-yl)methoxy)phenyl)acetic acid (130c)

Compound 130c was prepared according to the procedure reported step-2 of scheme-1 from ethyl 2-(2-((5-(1-aminoisoquinolin-5-yl)-2-(1-(ethylsulfonyl)azetidin-3-yl)-2H-indazol-3-yl)methoxy)phenyl)acetate (130b) (76 mg, 0.127 mmol) in THF (3 mL) using a solution of LiOH·H₂O (20.09 mg, 0.839 mmol) in water (1 mL) to afford after workup and purification using method-M, 2-(2-((5-(1-aminoisoquinolin-5-yl)-2-(1-(ethylsulfonyl)azetidin-3-yl)-2H-indazol-3-yl)methoxy)phenyl)acetic acid (130c) (37 mg, 23.15% yield) HCl salt as a white solid; 1H NMR (300 MHz, DMSO-d₆) δ 13.39 (s, 1H, D₂O) exchangeable), 12.18 (s, 1H, DO exchangeable), 9.24 (s, 2H, D₂O exchangeable), 8.65 (d, J=8.3 Hz, 1H), 8.02 (s, 1H), 7.97 (d, J=7.3 Hz, 1H), 7.93-7.83 (m, 2H), 7.66 (d, J=7.2 Hz, 1H), 7.45-7.34 (m, 1H), 7.34-7.26 (m, 2H), 7.22 (d, J=7.4 Hz, 1H), 7.02-6.91 (m, 2H), 5.84-5.71 (m, 1H), 5.65 (s, 2H), 4.64-4.50 (m, 2H), 4.41 (t, J=8.1 Hz, 2H), 3.48 (s, 2H), 3.32-3.26 (m, 2H), 1.32 (t, J=7.3 Hz, 3H); MS (ES+): 572.2 (M+1); (ES−): 570.1 (M−1).

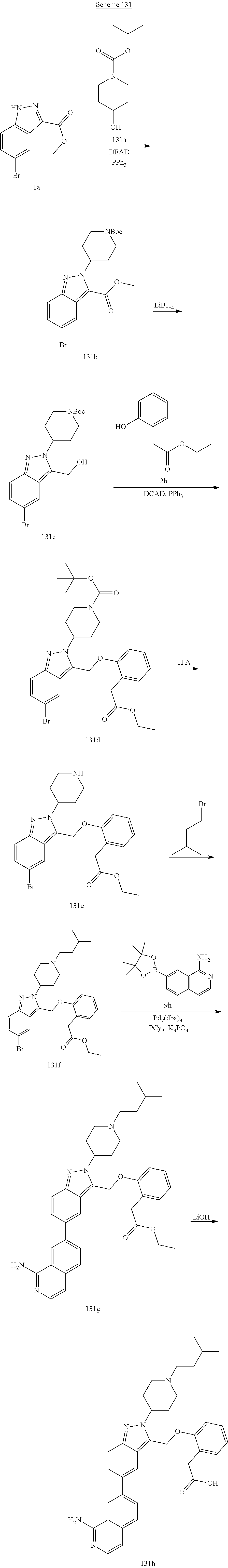

### Preparation of 2-(2-((5-(1-aminoisoquinolin-7-yl)-2-(1-isopentylpiperidin-4-yl)-2H-indazol-3-yl)methoxy)phenyl)acetic acid (131h)

### Step-1: Preparation of methyl 5-bromo-2-(1-(tert-butoxycarbonyl)piperidin-4-yl)-2H-indazole-3-carboxylate (131b)

Compound 131b was prepared according to the procedure reported in step-1 of scheme-8 from methyl 5-bromo-1H-indazole-3-carboxylate (1a) (9 g, 35.3 mmol) in THE using DEAD (9.83 g, 56.5 mmol), PPh₃ (14.81 g, 56.5 mmol) and tert-butyl 4-hydroxypiperidine-1-carboxylate (131a) (11.36 g, 56.5 mmol; CAS #109384-19-2) to afford after workup and purification using method-AR, methyl 5-bromo-2-(1-(tert-butoxycarbonyl)piperidin-4-yl)-2H-indazole-3-carboxylate (131b) (12.75 g, 82% yield) as yellow oil; ¹H NMR (300 MHZ, DMSO-d₆) δ 8.17 (dd, J=1.9, 0.8 Hz, 1H), 7.81 (dd, J=9.1, 0.7 Hz, 1H), 7.50 (dd, J=9.1, 1.9 Hz, 1H), 5.72-5.55 (m, 1H), 4.13 (d, J=13.2 Hz, 2H), 4.00 (s, 3H), 3.07-2.81 (m, 2H), 2.13-1.88 (m, 4H), 1.44 (s, 9H); MS (ES+): 460.0 and 462.1 (M+Na).

### Step-2: Preparation of tert-butyl 4-(5-bromo-3-(hydroxymethyl)-2H-indazol-2-yl)piperidine-1-carboxylate (131c)

Compound 131c was prepared according to the procedure reported in step-2 of scheme-119 from methyl 5-bromo-2-(1-(tert-butoxycarbonyl)piperidin-4-yl)-2H-indazole-3-carboxylate (131b) (6g, 13.69 mmol) in THF (30 mL) using LiBH₄ (4M in THF, 10.27 mL, 41.1 mmol) and MeOH (1.661 mL, 41.1 mmol) to afford after workup and purification using method-AR, tert-butyl 4-(5-bromo-3-(hydroxymethyl)-2H-indazol-2-yl)piperidine-1-carboxylate (131c) (5.5 g, 98% yield) as a clear oil; 1H NMR (300 MHz, DMSO-d₆) δ 8.04 (d, J=1.8 Hz, 1H), 7.57 (d, J=9.1 Hz, 1H), 7.30 (dd, J=9.1, 1.9 Hz, 1H), 5.50 (t, J=5.7 Hz, 1H), 4.93 (d, J=5.7 Hz, 2H), 4.84 (q, J=8.2 Hz, 1H), 4.19-4.06 (m, 2H), 3.10-2.79 (m, 2H), 2.02-1.95 (m, 4H), 1.44 (s, 9H); MS (ES+): 410.1 and 412.1 (M+1); (ES−): 444.0 and 446.0 (M+CI).

### Step-3: Preparation of tert-butyl 4-(5-bromo-3-((2-(2-ethoxy-2-oxoethyl)phenoxy)methyl)-2H-indazol-2-yl)piperidine-1-carboxylate (131d)

Compound 131d was prepared according to the procedure reported in step-2 of scheme-2, from tert-butyl 4-(5-bromo-3-(hydroxymethyl)-2H-indazol-2-yl)piperidine-1-carboxylate (131c) (5.5 g, 13.40 mmol) in DCM (100 mL), using PPh₃ (3.87 g, 14.75 mmol), ethyl 2-(2-hydroxyphenyl)acetate (2b) (2.66 g, 14.75 mmol) and a solution of DCAD (5.41 g, 14.75 mmol) in DCM (20 mL) to afford after workup and purification using method-K, tert-butyl 4-(5-bromo-3-((2-(2-ethoxy-2-oxoethyl)phenoxy)methyl)-2H-indazol-2-yl)piperidine-1-carboxylate (131d) (3.9 g, 50.8% yield) as yellow oil; MS (ES+): 572.2 and 574.2 (M+1), 594.1 and 596.1 (M+Na).

### Step-4: Preparation of ethyl 2-(2-((5-bromo-2-(piperidin-4-yl)-2H-indazol-3-yl)methoxy)phenyl)acetate (131e)

Compound 131e was prepared according to the procedure reported in step-4 of scheme-9 from tert-butyl 4-(5-bromo-3-((2-(2-ethoxy-2-oxoethyl)phenoxy)methyl)-2H-indazol-2-yl)piperidine-1-carboxylate (131d) (3.9 g, 6.81 mmol, 50.8% yield) in DCM (30 mL) using TFA (10.33 mL, 134 mmol) to afford after workup and purification using method-O, ethyl 2-(2-((5-bromo-2-(piperidin-4-yl)-2H-indazol-3-yl)methoxy)phenyl)acetate (131e) (2.2 g, 34.7% yield) as a clear oil; MS (ES+): 472.1 and 474.1 (M+1)

### Step-5: Preparation of ethyl 2-(2-((5-bromo-2-(1-isopentylpiperidin-4-yl)-2H-indazol-3-yl)methoxy)phenyl)acetate (131f)

Compound 131f was prepared according to the procedure reported in step-3 of scheme-106 from ethyl 2-(2-((5-bromo-2-(piperidin-4-yl)-2H-indazol-3-yl)methoxy)phenyl)acetate (131e) (500 mg, 1.058 mmol) in ACN (10 mL) using 1-bromo-3-methylbutane (192 mg, 1.270 mmol) and K₂CO₃ (366 mg, 2.65 mmol) to afford after workup and purification using method-P, ethyl 2-(2-((5-bromo-2-(1-isopentylpiperidin-4-yl)-2H-indazol-3-yl)methoxy)phenyl)acetate (131f) (320 mg, 55.7% yield) as yellow oil; MS (ES+): 542.2 and 544.2 (M+1); (ES−): 540.1 and 542.1 (M−1).

### Step-6: Preparation of ethyl 2-(2-((5-(1-aminoisoquinolin-7-yl)-2-(1-isopentylpiperidin-4-yl)-2H-indazol-3-yl)methoxy)phenyl)acetate (131g)

Compound 131g was prepared according to the procedure reported in step-3 of scheme-112 from ethyl 2-(2-((5-bromo-2-(1-isopentylpiperidin-4-yl)-2H-indazol-3-yl)methoxy)phenyl)acetate (131f) (160 mg, 0.295 mmol) in dioxane/2-MeTHF (4 mL, 3:1) using 7-(4,4,5,5-tetramethyl-1,3,2-dioxaborolan-2-yl)isoquinolin-1-amine (9h) (120 mg, 0.442 mmol), K₃PO₄ (2M aqueous solution, 0.221 mL, 0.885 mmol), PCy₃ (33.1 mg, 0.118 mmol) and Pd₂(dba)₃ (54.0 mg, 0.059 mmol) to afford after workup and purification using method-U, ethyl 2-(2-((5-(1-aminoisoquinolin-7-yl)-2-(1-isopentylpiperidin-4-yl)-2H-indazol-3-yl)methoxy)phenyl)acetate (131g) (76 mg, 42.5% yield) as a clear oil; MS (ES+): 606.3 (M+1); (ES−): 604.3 (M−1).

### Step-7: Preparation of 2-(2-((5-(1-aminoisoquinolin-7-yl)-2-(1-isopentylpiperidin-4-yl)-2H-indazol-3-yl)methoxy)phenyl)acetic acid (131h)

Compound 131h was prepared according to the procedure reported step-2 of scheme-1 from ethyl 2-(2-((5-(1-aminoisoquinolin-7-yl)-2-(1-isopentylpiperidin-4-yl)-2H-indazol-3-yl)methoxy)phenyl)acetate (131g) (76 mg, 0.125 mmol, 42.5% yield) in THF (3 mL) using a solution of LiOH·H₂O (21.19 mg, 0.885 mmol) in water (1 mL) to afford after workup and purification using method-M, 2-(2-((5-(1-aminoisoquinolin-7-yl)-2-(1-isopentylpiperidin-4-yl)-2H-indazol-3-yl)methoxy)phenyl)acetic acid (131h) (36 mg, 21.13% yield) HCl salt as a white solid; ¹H NMR (300 MHz, DMSO-d₆) δ 13.47 (s, 1H, DO exchangeable), 12.16 (s, 1H, D₂O exchangeable), 10.73 (s, 1H, D₂O exchangeable), 9.30 (s, 2H, D₂O exchangeable), 9.05 (s, 1H), 8.55 (s, 1H), 8.46 (d, J=8.5 Hz, 1H), 8.05 (d, J=8.5 Hz, 1H), 7.95 (d, J=9.2 Hz, 1H), 7.88 (d, J=9.0 Hz, 1H), 7.70 (d, J=6.9 Hz, 1H), 7.43-7.34 (m, 2H), 7.31-7.20 (m, 2H), 7.05-6.93 (m, 1H), 5.72 (s, 2H), 5.05-4.84 (m, 1H), 3.74-3.61 (m, 2H), 3.50 (s, 2H), 3.27-3.00 (m, 4H), 2.79-2.61 (m, 2H), 2.31-2.17 (m, 2H), 1.74-1.55 (m, 3H), 0.92 (d, J=5.8 Hz, 6H); MS (ES+): 578.3 (M+1); (ES−): 576.2 (M−1).

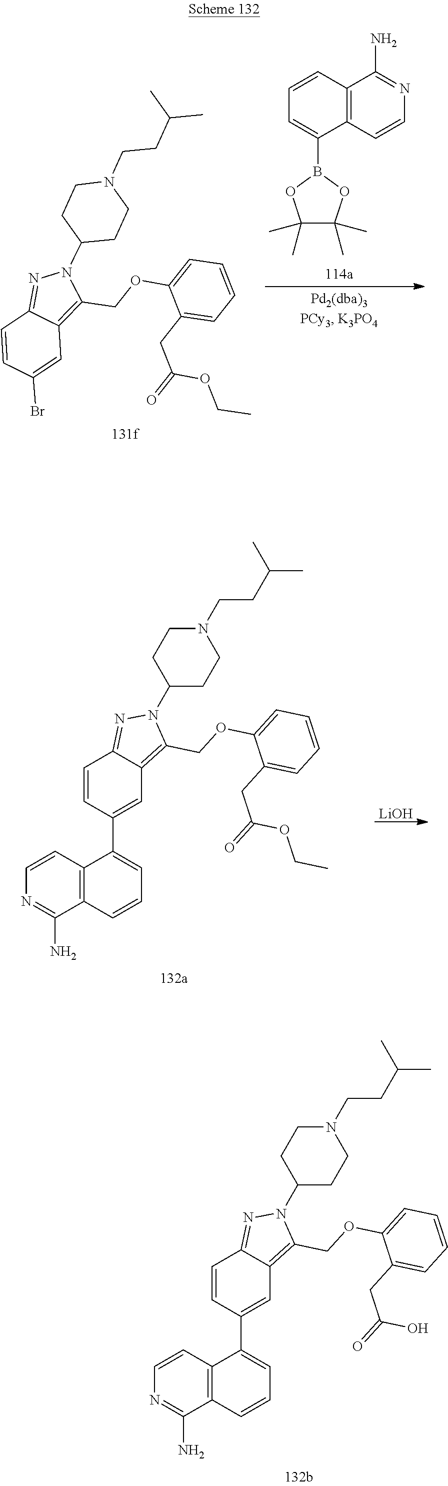 [SMILES: CCOC(=O)Cc1ccccc1OCc1c2cc(Br)ccc2nn1C1CCN(CCC(C)C)CC1 ; CC1(C)OB(c2cccc3c(N)nccc23)OC1(C)C ; CCOC(=O)Cc1ccccc1OCc1c2cc(-c3cccc4c(N)nccc34)ccc2nn1C1CCN(CCC(C)C)CC1 ; "LiOH" ; CC(C)CCN1CCC(n2nc3ccc(-c4cccc5c(N)nccc45)cc3c2COc2ccccc2CC(=O)O)CC1]

### Preparation of 2-(2-((5-(1-aminoisoquinolin-5-yl)-2-(1-isopentylpiperidin-4-yl)-2H-indazol-3-yl)methoxy)phenyl)acetic acid (132b)

### Step-1: Preparation of ethyl 2-(2-((5-(1-aminoisoquinolin-5-yl)-2-(1-isopentylpiperidin-4-yl)-2H-indazol-3-yl)methoxy)phenyl)acetate (132a)

Compound 132a was prepared according to the procedure reported in step-3 of scheme-112 from ethyl 2-(2-((5-bromo-2-(1-isopentylpiperidin-4-yl)-2H-indazol-3-yl)methoxy)phenyl)acetate (131f) (160 mg, 0.295 mmol) in dioxane/2-MeTHF (4 mL, 3:1) using 5-(4,4,5,5-tetramethyl-1,3,2-dioxaborolan-2-yl)isoquinolin-1-amine (114a) (120 mg, 0.442 mmol), K₃PO₄ (2M aqueous solution, 0.221 mL, 0.885 mmol), PCy₃ (33.1 mg, 0.118 mmol) and Pd₂(dba)₃ (54.0 mg, 0.059 mmol) to afford after workup and purification using method-U, ethyl 2-(2-((5-(1-aminoisoquinolin-5-yl)-2-(1-isopentylpiperidin-4-yl)-2H-indazol-3-yl)methoxy)phenyl)acetate (132a) (63 mg, 35.3% yield) as a clear oil; MS (ES+): 606.4 (M+1).

### Step-2: Preparation of 2-(2-((5-(1-aminoisoquinolin-5-yl)-2-(1-isopentylpiperidin-4-yl)-2H-indazol-3-yl)methoxy)phenyl)acetic acid (132b)

Compound 132b was prepared according to the procedure reported step-2 of scheme-1 from ethyl 2-(2-((5-(1-aminoisoquinolin-5-yl)-2-(1-isopentylpiperidin-4-yl)-2H-indazol-3-yl)methoxy)phenyl)acetate (132a) (63 mg, 0.104 mmol) in THF (3 mL) using a solution of LiOH·H₂O (21.19 mg, 0.885 mmol) in water (1 mL) to afford after workup and purification using method-M, 2-(2-((5-(1-aminoisoquinolin-5-yl)-2-(1-isopentylpiperidin-4-yl)-2H-indazol-3-yl)methoxy)phenyl)acetic acid (132b) (15 mg, 8.80% yield) HCl salt as a white solid; JH NMR (300 MHz, DMSO-d₆) δ 13.41 (s, 1H, D₂O exchangeable), 12.15 (s, 1H, D₂O exchangeable), 10.55 (s, 1H, D₂O exchangeable), 9.11 (s, 2H, D₂O exchangeable), 8.63 (d, J=8.3 Hz, 1H), 8.02 (s, 1H), 7.94 (d, J=7.2 Hz, 1H), 7.89-7.79 (m, 2H), 7.66 (d, J=7.1 Hz, 1H), 7.40-7.27 (m, 3H), 7.23 (d, J=7.4 Hz, 1H), 7.03-6.91 (m, 2H), 5.66 (s, 2H), 5.05-4.89 (m, 1H), 3.75-3.59 (m, 2H), 3.49 (s, 2H), 3.24-2.99 (m, 4H), 2.78-2.62 (m, 2H), 2.32-2.18 (m, 2H), 1.75-1.55 (m, 3H), 0.93 (d, J=5.6 Hz, 6H); MS (ES+): 578.3 (M+1); (ES−): 576.3 (M−1).

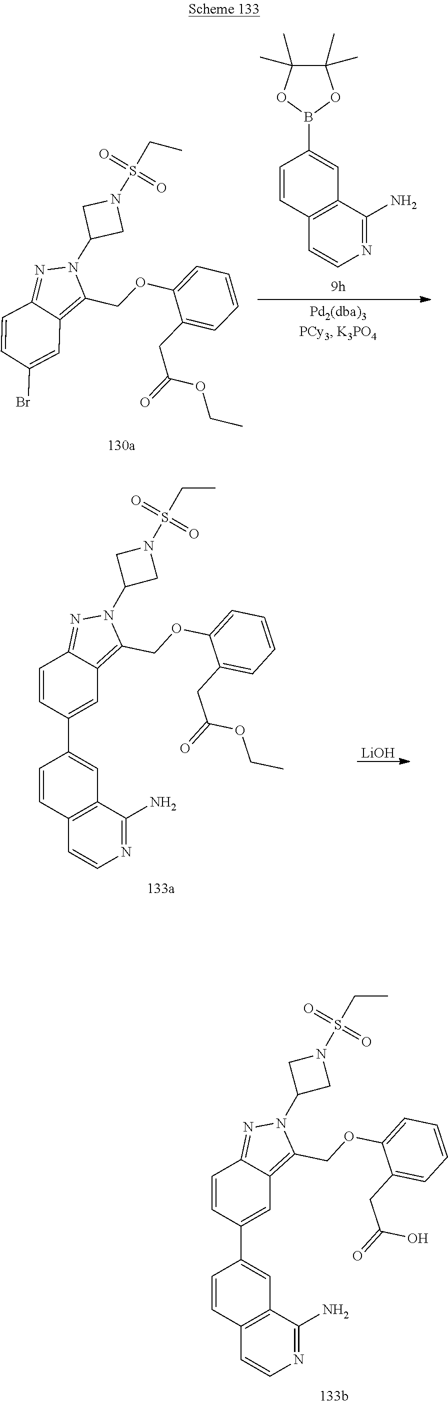 [SMILES: CCOC(=O)Cc1ccccc1OCc1c2cc(Br)ccc2nn1C1CN(S(=O)(=O)CC)C1 ; CC1(C)OB(c2ccc3ccnc(N)c3c2)OC1(C)C ; CCOC(=O)Cc1ccccc1OCc1c2cc(-c3ccc4ccnc(N)c4c3)ccc2nn1C1CN(S(=O)(=O)CC)C1 ; "LiOH" ; CCS(=O)(=O)N1CC(n2nc3ccc(-c4ccc5ccnc(N)c5c4)cc3c2COc2ccccc2CC(=O)O)C1]

### Preparation of 2-(2-((5-(1-aminoisoquinolin-7-yl)-2-(1-(ethylsulfonyl)azetidin-3-yl)-2H-indazol-3-yl)methoxy)phenyl)acetic acid (133b)

### Step-1: Preparation of ethyl 2-(2-((5-(1-aminoisoquinolin-7-yl)-2-(1-(ethylsulfonyl)azetidin-3-yl)-2H-indazol-3-yl)methoxy)phenyl)acetate (133a)

Compound 133a was prepared according to the procedure reported in step-3 of scheme-112 from ethyl 2-(2-((5-bromo-2-(1-(ethylsulfonyl)azetidin-3-yl)-2H-indazol-3-yl)methoxy)phenyl)acetate (130a) (135 mg, 0.252 mmol) in dioxane/2-MeTHF (4 mL, 3:1) using 7-(4,4,5,5-tetramethyl-1,3,2-dioxaborolan-2-yl)isoquinolin-1-amine (9h) (68.0 mg, 0.252 mmol), K₃PO₄ (2M aqueous solution, 0.189 mL, 0.755 mmol), PCy₃ (21.17 mg, 0.075 mmol) and Pd₂(dba)₃ (46.1 mg, 0.05 mmol) to afford after workup and purification using method-U, ethyl 2-(2-((5-(1-aminoisoquinolin-7-yl)-2-(1-(ethylsulfonyl)azetidin-3-yl)-2H-indazol-3-yl)methoxy)phenyl)acetate (133a) (58 mg, 38.4% yield) as a clear oil; MS (ES+): 600.2 (M+1).

### Step-2: Preparation of 2-(2-((5-(1-aminoisoquinolin-7-yl)-2-(1-(ethylsulfonyl)azetidin-3-yl)-2H-indazol-3-yl)methoxy)phenyl)acetic acid (133b)

Compound 133b was prepared according to the procedure reported step-2 of scheme-1 from ethyl 2-(2-((5-(1-aminoisoquinolin-7-yl)-2-(1-(ethylsulfonyl)azetidin-3-yl)-2H-indazol-3-yl)methoxy)phenyl)acetate (133a) (58 mg, 0.097 mmol, 38.4% yield) in THF (3 mL) using a solution of LiOH·H₂O (18.08 mg, 0.755 mmol) in water (1 mL) to afford after workup and purification using method-M, 2-(2-((5-(1-aminoisoquinolin-7-yl)-2-(1-(ethylsulfonyl)azetidin-3-yl)-2H-indazol-3-yl)methoxy)phenyl)acetic acid (133b) (17 mg, 11.82% yield) HCl salt as a white solid; 1H NMR (300 MHz, DMSO-d₆) δ 13.22 (s, 1H, D₂O exchangeable), 12.17 (s, 1H, D₂O exchangeable), 9.13 (s, 1H, D₂O exchangeable), 8.99 (s, 1H), 8.49 (s, 1H), 8.48-8.42 (m, 1H), 8.05 (d, J=8.5 Hz, 1H), 8.01-7.89 (m, 2H), 7.69 (d, J=6.9 Hz, 1H), 7.39-7.30 (m, 2H), 7.25 (t, J=8.0 Hz, 2H), 7.03-6.92 (m, 1H), 5.81-5.71 (m, 1H), 5.68 (s, 2H), 4.54 (t, J=7.2 Hz, 2H), 4.40 (t, J=8.1 Hz, 2H), 3.48 (s, 2H), 3.30-3.24 (m, 2H), 1.31 (t, J=7.3 Hz, 3H); MS (ES+): 572.2 (M+1); (ES−): 570.1 (M−1).

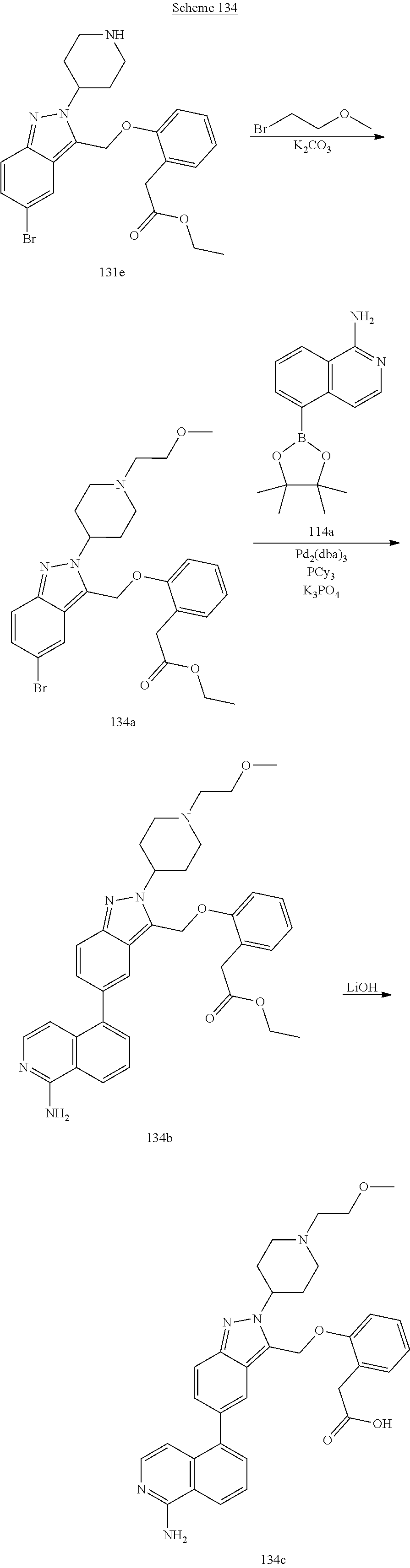

### Preparation of 2-(2-((5-(1-aminoisoquinolin-5-yl)-2-(1-(2-methoxyethyl)piperidin-4-yl)-2H-indazol-3-yl)methoxy)phenyl)acetic acid (134c)

### Step-1: Preparation of ethyl 2-(2-((5-bromo-2-(1-(2-methoxyethyl)piperidin-4-yl)-2H-indazol-3-yl)methoxy)phenyl)acetate (134a)

Compound 134a was prepared according to the procedure reported in step-3 of scheme-106 from ethyl 2-(2-((5-bromo-2-(piperidin-4-yl)-2H-indazol-3-yl)methoxy)phenyl)acetate (131e) (500 mg, 1.058 mmol) in DMF (6 mL) using 1-bromo-2-methoxyethane (177 mg, 1.270 mmol) and K₂CO₃ (366 mg, 2.65 mmol) to afford after workup and purification using method-P, ethyl 2-(2-((5-bromo-2-(1-(2-methoxyethyl)piperidin-4-yl)-2H-indazol-3-yl)methoxy)phenyl)acetate (134a) (96 mg, 17.10% yield) as yellow oil; MS (ES+): 530.2 and 532.2 (M+1).

### Step-2: Preparation of ethyl 2-(2-((5-(1-aminoisoquinolin-5-yl)-2-(1-(2-methoxyethyl)piperidin-4-yl)-2H-indazol-3-yl)methoxy)phenyl)acetate (134b)

Compound 134b was prepared according to the procedure reported in step-3 of scheme-112 from ethyl 2-(2-((5-bromo-2-(1-(2-methoxyethyl)piperidin-4-yl)-2H-indazol-3-yl)methoxy)phenyl)acetate (134a) (96 mg, 0.181 mmol) in dioxane/2-MeTHF (4 mL, 3:1) using 5-(4,4,5,5-tetramethyl-1.3,2-dioxaborolan-2-yl)isoquinolin-1-amine (114a) (73.3 mg, 0.271 mmol), K₃PO₄ (2M aqueous solution, 0.136 mL, 0.543 mmol), PCy₃ (15.23 mg, 0.054 mmol) and Pd₂(dba)₃ (33.1 mg, 0.036 mmol) to afford after workup and purification using method-U, ethyl 2-(2-((5-(1-aminoisoquinolin-5-yl)-2-(1-(2-methoxyethyl)piperidin-4-yl)-2H-indazol-3-yl)methoxy)phenyl)acetate (134b) (65 mg, 60.5% yield) as a clear oil; MS (ES+): 594.3 (M+1).

### Step-3: Preparation of 2-(2-((5-(1-aminoisoquinolin-5-yl)-2-(1-(2-methoxyethyl)piperidin-4-yl)-2H-indazol-3-yl)methoxy)phenyl)acetic acid (134c)

Compound 134c was prepared according to the procedure reported step-2 of scheme-1 from ethyl 2-(2-((5-(1-aminoisoquinolin-5-yl)-2-(1-(2-methoxyethyl)piperidin-4-yl)-2H-indazol-3-yl)methoxy)phenyl)acetate (134b) (65 mg, 0.109 mmol, 60.5% yield) in THF (3 mL) using a solution of LiOH·H₂O (13.0 mg, 0.543 mmol) in water (1 mL) to afford after workup and purification using method-M, 2-(2-((5-(1-aminoisoquinolin-5-yl)-2-(1-(2-methoxyethyl)piperidin-4-yl)-2H-indazol-3-yl)methoxy)phenyl)acetic acid (134c) (18 mg, 17.58% yield) HCl salt as a white solid, 1H NMR (300 MHz, DMSO-d₆) δ 13.42 (s, 1H, D₂O exchangeable), 12.10 (s, 1H, D₂O exchangeable), 10.55 (s, 1H, D₂O exchangeable), 9.26 (s, 2H, D₂O exchangeable), 8.65 (d, J=8.3 Hz, 1H), 8.02 (s, 1H), 7.96 (d, J=7.3 Hz, 1H), 7.92-7.79 (m, 2H), 7.65 (d, J=7.2 Hz, 1H), 7.41-7.26 (m, 3H), 7.23 (d, J=7.4 Hz, 1H), 7.03-6.89 (m, 2H), 5.66 (s, 2H), 5.05-4.88 (m, 1H), 3.77 (t, J=4.6 Hz, 2H), 3.68 (d, J=12.0 Hz, 2H), 3.48 (s, 2H), 3.34-3.32 (m, 5H), 3.30-3.17 (m, 2H), 2.79-2.60 (m, 2H), 2.31-2.17 (m, 2H); MS (ES+): 566.2; (ES−): 564.2 (M−1).

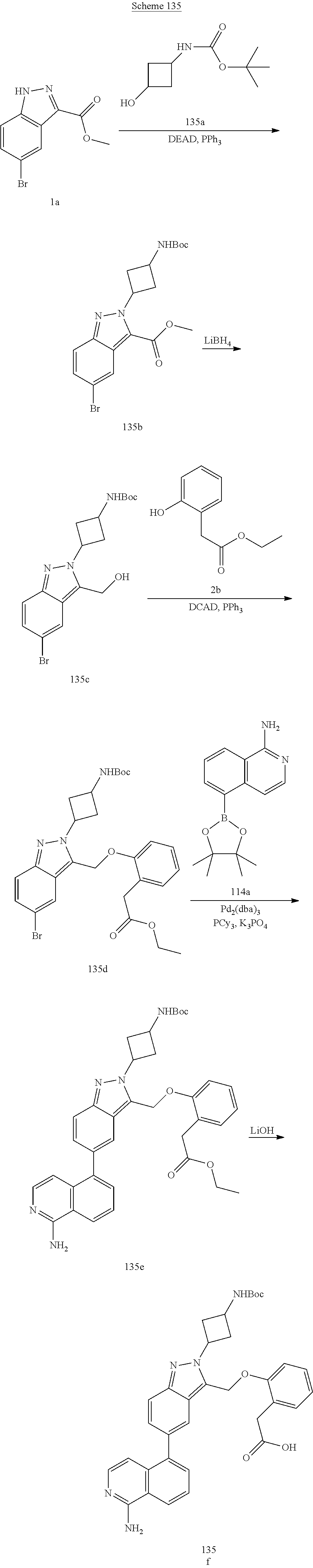

### Preparation of 2-(2-((5-(1-aminoisoquinolin-5-yl)-2-(3-(tert-butoxycarbonylamino)cyclobutyl)-2H-indazol-3-yl)methoxy)phenyl)acetic acid (135f)

### Step-1: Preparation of methyl 5-bromo-2-(3-(tert-butoxycarbonylamino)cyclobutyl)-2H-indazole-3-carboxylate (135b)

Compound 135b was prepared according to the procedure reported in step-1 of scheme-8 from methyl 5-bromo-1H-indazole-3-carboxylate (1a) (8 g, 31.4 mmol) in THF using DEAD (10.92 g, 62.7 mmol), PPh₃ (16.45 g, 62.7 mmol), tert-butyl 3-hydroxycyclobutylcarbamate (135a) (11.75 g, 62.7 mmol; CAS #154748-63-7) to afford after workup and purification using method-AR, methyl 5-bromo-2-(3-(tert-butoxycarbonylamino)cyclobutyl)-2H-indazole-3-carboxylate (135b) (10.5 g, 79% yield) as white solid; MS (ES+): 446.1 and 448.0 (M+Na).

### Step-2: Preparation of tert-butyl 3-(5-bromo-3-(hydroxymethyl)-2H-indazol-2-yl)cyclobutyl carbamate (135c)

Compound 135c was prepared according to the procedure reported in step-2 of scheme-119 from methyl 5-bromo-2-(3-(tert-butoxycarbonylamino)cyclobutyl)-2H-indazole-3-carboxylate (135b) (10.5 g, 24.75 mmol) in THF (100 mL) using LiBH₄ (4M in THF, 18.56 mL, 74.2 mmol) and MeOH (3.00 mL, 74.2 mmol) to afford after workup and purification using method-AS, tert-butyl 3-(5-bromo-3-(hydroxymethyl)-2H-indazol-2-yl)cyclobutyl carbamate (135c) (8 g, 82% yield) as a white solid; MS (ES+): 396.1 and 398.0 (M+1).

### Step-3: Preparation of ethyl 2-(2-((5-bromo-2-(3-(tert-butoxycarbonylamino)cyclobutyl)-2H-indazol-3-yl)methoxy)phenyl)acetate (135d)

Compound 135d was prepared according to the procedure reported in step-2 of scheme-2, from tert-butyl 3-(5-bromo-3-(hydroxymethyl)-2H-indazol-2-yl)cyclobutyl carbamate (135c) (6.5 g, 16.4 mmol) in DCM (100 mL) using PPh₃ (4.73 g, 18.04 mmol), ethyl 2-(2-hydroxyphenyl)acetate (2b) (3.25 g, 18.04 mmol) and a solution of DCAD (6.63 g, 18.04 mmol) in DCM (20 mL) to afford after workup and purification using method-K, ethyl 2-(2-((5-bromo-2-(3-(tert-butoxycarbonylamino)cyclobutyl)-2H-indazol-3-yl)methoxy)phenyl)acetate (135d) (6.1 g, 66.6% yield) as yellow oil; MS (ES+): 558.2 and 560.2 (M+1).

### Step-4: Preparation of ethyl 2-(2-((5-(1-aminoisoquinolin-5-yl)-2-(3-(tert-butoxycarbonylamino)cyclobutyl)-2H-indazol-3-yl)methoxy)phenyl)acetate (135e)

Compound 135e was prepared according to the procedure reported in step-3 of scheme-112 from ethyl 2-(2-((5-bromo-2-(3-(tert-butoxycarbonylamino)cyclobutyl)-2H-indazol-3-yl)methoxy)phenyl)acetate (135d) (250 mg, 0.448 mmol) in dioxane/2-MeTHF (4 mL, 3:1) using 5-(4,4,5,5-tetramethyl-1,3,2-dioxaborolan-2-yl)isoquinolin-1-amine (114a) (181 mg, 0.671 mmol), K₃PO₄ (2M aqueous solution, 0.336 mL, 1.343 mmol), PCy₃ (50.2 mg, 0.179 mmol) and Pd₂(dba)₃ (82 mg, 0.090 mmol) to afford after workup and purification using method-U, ethyl 2-(2-((5-(1-aminoisoquinolin-5-yl)-2-(3-(tert-butoxycarbonylamino)cyclobutyl)-2H-indazol-3-yl)methoxy)phenyl)acetate (135e) (168 mg, 60.4% yield) as a clear oil; MS (ES+): 622.3 (M+1).

### Step-5, Preparation of 2-(2-((5-(1-aminoisoquinolin-5-yl)-2-(3-(tert-butoxycarbonylamino)cyclobutyl)-2H-indazol-3-yl)methoxy)phenyl)acetic acid (135f)

Compound 135f was prepared according to the procedure reported step-2 of scheme-1 from ethyl 2-(2-((5-(1-aminoisoquinolin-5-yl)-2-(3-(tert-butoxycarbonylamino)cyclobutyl)-2H-indazol-3-yl)methoxy)phenyl)acetate (135e) (84 mg, 0.135 mmol) in THF (3 mL) using a solution of LiOH·H₂O (32.2 mg, 1.343 mmol) in water (1 mL) to afford after workup and purification using method-M, 2-(2-((5-(1-aminoisoquinolin-5-yl)-2-(3-(tert-butoxycarbonylamino)cyclobutyl)-2H-indazol-3-yl)methoxy)phenyl)acetic acid (135f) (41 mg, 30% yield) HCl salt as a white solid; 1H NMR (300 MHz, DMSO-d₆) δ 13.35 (s, 1H, D₂O exchangeable), 12.16 (s, 1H, D₂O exchangeable), 9.22 (s, 2H, D₂O exchangeable), 8.63 (d, J=8.3 Hz, 1H), 7.96 (d, J=6.3 Hz, 2H), 7.89-7.80 (m, 2H), 7.65 (d, J=7.2 Hz, 1H), 7.42 (d, J=7.8 Hz, 1H, D₂O exchangeable), 7.33 (d, J=8.9 Hz, 1H), 7.27 (d, J=4.2 Hz, 2H), 7.21 (d, J=7.4 Hz, 1H), 7.03-6.89 (m, 2H), 5.67-5.51 (m, 2H), 5.09-4.94 (m, 1H), 3.99-3.83 (m, 1H), 3.47 (d, J=6.1 Hz, 2H), 2.91-2.57 (m, 4H), 1.40 (s, 9H); MS (ES+) 594.2 (M+1); (ES−) 592.2 (M−1).

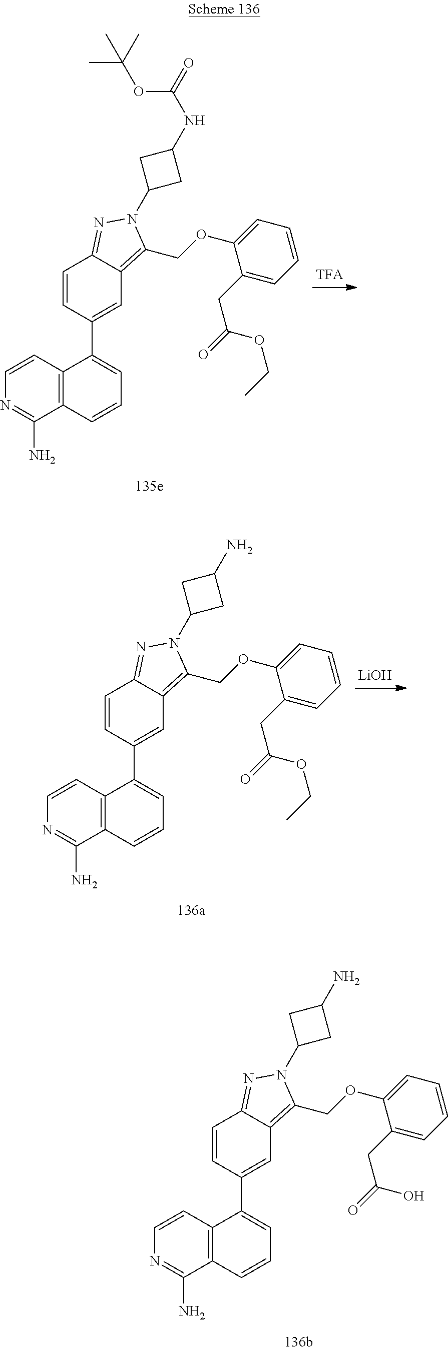

### Preparation of 2-(2-((2-(3-aminocyclobutyl)-5-(1-aminoisoquinolin-5-yl)-2H-indazol-3-yl)methoxy)phenyl)acetic acid (136b)

### Step-1: Preparation of ethyl 2-(2-((2-(3-aminocyclobutyl)-5-(1-aminoisoquinolin-5-yl)-2H-indazol-3-yl)methoxy)phenyl)acetate (136a)

Compound 136a was prepared according to the procedure reported in step-4 of scheme-9 from ethyl 2-(2-((5-(1-aminoisoquinolin-5-yl)-2-(3-(tert-butoxycarbonylamino)cyclobutyl)-2H-indazol-3-yl)methoxy)phenyl)acetate (135e) (84 mg, 0.135 mmol) in DCM (4 mL) using TFA (0.104 mL, 1.351 mmol) to afford after workup ethyl 2-(2-((2-(3-aminocyclobutyl)-5-(1-aminoisoquinolin-5-yl)-2H-indazol-3-yl)methoxy)phenyl)acetate (136a) (68 mg, 96% yield) as a yellow oil, and was used as such for the next step; MS (ES+): 522.2 (M+1).

### Step-2: Preparation of 2-(2-((2-(3-aminocyclobutyl)-5-(1-aminoisoquinolin-5-yl)-2H-indazol-3-yl)methoxy)phenyl)acetic acid (136b)

Compound 136b was prepared according to the procedure reported step-2 of scheme-1 from ethyl 2-(2-((2-(3-aminocyclobutyl)-5-(1-aminoisoquinolin-5-yl)-2H-indazol-3-yl)methoxy)phenyl)acetate (136a) (68 mg, 0.130 mmol) in THF (3 mL) using a solution of LiOH·H₂O) (9.71 mg, 0.405 mmol) in water (1 mL) to afford after workup and purification using method-M, 2-(2-((2-(3-aminocyclobutyl)-5-(1-aminoisoquinolin-5-yl)-2H-indazol-3-yl)methoxy)phenyl)acetic acid (136b) (42 mg, 63.0% yield) HCl salt as a white solid; ¹H NMR (300 MHz, DMSO-d₆) δ 13.74 (s, 1H, D₂O exchangeable), 9.43 (s, 2H, D₂O exchangeable), 8.73 (d, J=8.4 Hz, 1H), 8.70-8.55 (m, 3H, D₂O exchangeable), 8.06-7.90 (m, 2H), 7.90-7.79 (m, 2H), 7.74-7.62 (m, 1H), 7.36 (dd, J=8.9, 1.9 Hz, 1H), 7.28 (d, J=4.4 Hz, 2H), 7.25-7.15 (m, 1H), 7.00-6.90 (m, 2H), 5.60 (d, J=4.2 Hz, 2H), 5.28-5.13 (m, 1H), 3.75-3.59 (m, 1H), 3.49 (d, J=4.7 Hz, 2H), 3.01-2.82 (m, 4H); MS (ES+): 494.2 (M+1); (ES−): 492.1 (M−1).

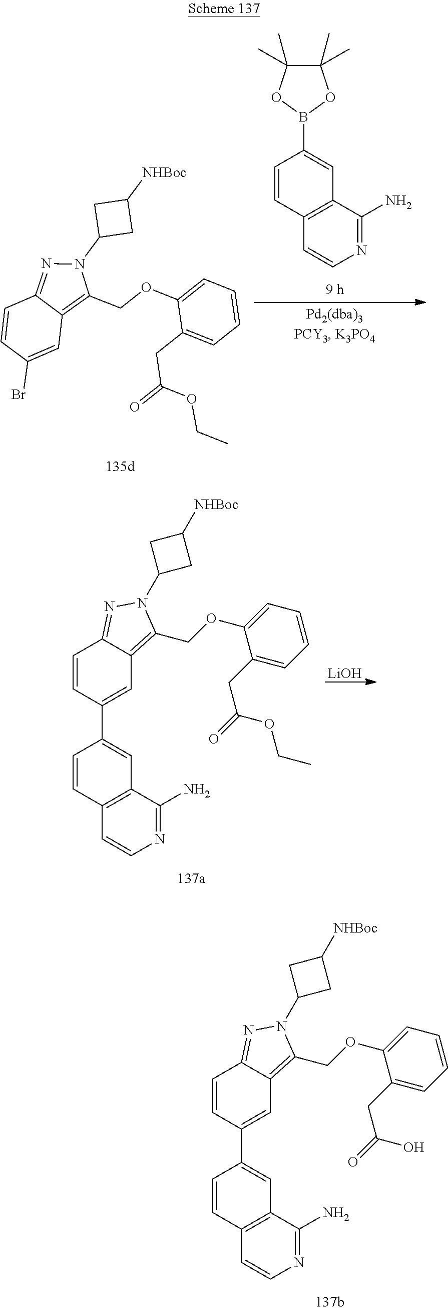

### Preparation of 2-(2-((5-(1-aminoisoquinolin-7-yl)-2-(3-(tert-butoxycarbonylamino)cyclobutyl)-2H-indazol-3-yl)methoxy)phenyl)acetic acid (137b)

### Step-1: Preparation of ethyl 2-(2-((5-(1-aminoisoquinolin-7-yl)-2-(3-(tert-butoxycarbonylamino)cyclobutyl)-2H-indazol-3-yl)methoxy)phenyl)acetate (137a)

Compound 137a was prepared according to the procedure reported in step-3 of scheme-112 from ethyl 2-(2-((5-bromo-2-(3-(tert-butoxycarbonylamino)cyclobutyl)-2H-indazol-3-yl)methoxy)phenyl)acetate (135d) (250 mg, 0.448 mmol) in dioxane/2-MeTHF (4 mL, 3:1) using 7-(4,4,5,5-tetramethyl-1,3,2-dioxaborolan-2-yl)isoquinolin-1-amine (9h) (181 mg, 0.671 mmol), K₃PO₄ (2M aqueous solution, 0.336 mL, 1.343 mmol), PCy₃ (37.7 mg, 0.134 mmol) and Pd₂(dba)₃ (82 mg, 0.09 mmol) to afford after workup and purification using method-U, ethyl 2-(2-((5-(1-aminoisoquinolin-7-yl)-2-(3-(tert-butoxycarbonylamino)cyclobutyl)-2H-indazol-3-yl)methoxy)phenyl)acetate (137a) (160 mg, 57.5% yield) as a clear oil; MS (ES+): 622.3 (M+1).

### Step-2: Preparation of 2-(2-((5-(1-aminoisoquinolin-7-yl)-2-(3-(tert-butoxycarbonylamino)cyclobutyl)-2H-indazol-3-yl)methoxy)phenyl)acetic acid (137b)

Compound 137b was prepared according to the procedure reported step-2 of scheme-1 from ethyl 2-(2-((5-(1-aminoisoquinolin-7-yl)-2-(3-(tert-butoxycarbonylamino)cyclobutyl)-2H-indazol-3-yl)methoxy)phenyl)acetate (137a) (80 mg, 0.129 mmol) in THF (3 mL) using a solution of LiOH·H₂O (32.2 mg, 1.343 mmol) in water (1 mL) to afford after workup and purification using method-M, 2-(2-((5-(1-aminoisoquinolin-7-yl)-2-(3-(tert-butoxycarbonylamino)cyclobutyl)-2H-indazol-3-yl)methoxy)phenyl)acetic acid (137b) (43 mg, 16.18% yield) HCl salt as a white solid; 1H NMR (300 MHz, DMSO-d₆) δ 13.29 (s, 1H, D₂O exchangeable), 12.18 (s, 1H, D₂O exchangeable), 9.21 (s, 2H, DO exchangeable), 8.99 (s, 1H), 8.50-8.41 (m, 2H), 8.05 (d, J=8.5 Hz, 1H), 7.94-7.82 (m, 2H), 7.68 (d, J=6.9 Hz, 1H), 7.41 (d, J=7.8 Hz, 1H), 7.36-7.30 (m, 2H), 7.30-7.21 (m, 2H), 7.03-6.90 (m, 1H), 5.69-5.57 (m, 2H), 5.42-5.32 (m, 1H), 5.07-4.91 (m, 1H), 4.57-4.45 (m, 1H), 3.99-3.83 (m, 1H), 3.51-3.47 (m, 2H), 2.85-2.60 (m, 4H), 1.40 (s, 9H); MS (ES+): 594.3 (M+1); (ES−): 592.2 (M−1).

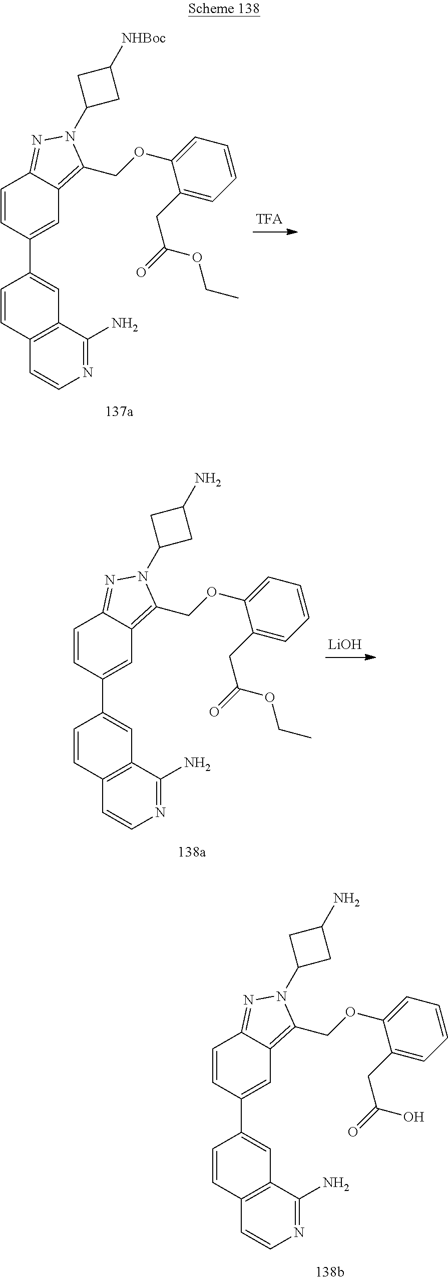

### Preparation of 2-(2-((2-(3-aminocyclobutyl)-5-(1-aminoisoquinolin-7-yl)-2H-indazol-3-yl)methoxy)phenyl)acetic acid (138b)

### Step-1: Preparation of ethyl 2-(2-((2-(3-aminocyclobutyl)-5-(1-aminoisoquinolin-7-yl)-2H-indazol-3-yl)methoxy)phenyl)acetate (138a)

Compound 138a was prepared according to the procedure reported in step-4 of scheme-9 from ethyl 2-(2-((5-(1-aminoisoquinolin-7-yl)-2-(3-(tert-butoxycarbonylamino)cyclobutyl)-2H-indazol-3-yl)methoxy)phenyl)acetate (137a) (80 mg, 0.129 mmol) in DCM (4 mL) using TFA (0.099 mL, 1.287 mmol) to afford after workup ethyl 2-(2-((2-(3-aminocyclobutyl)-5-(1-aminoisoquinolin-7-yl)-2H-indazol-3-yl)methoxy)phenyl)acetate (138a) (48 mg, 71.5% yield) as a yellow oil; MS (ES+): 522.2 (M+1).

### Step-2: Preparation of 2-(2-((2-(3-aminocyclobutyl)-5-(1-aminoisoquinolin-7-yl)-2H-indazol-3-yl)methoxy)phenyl)acetic acid (138b)

Compound 138b was prepared according to the procedure reported step-2 of scheme-1 from ethyl 2-(2-((2-(3-aminocyclobutyl)-5-(1-aminoisoquinolin-7-yl)-2H-indazol-3-yl)methoxy)phenyl)acetate (138a) in THF (3 mL) using a solution of LiOH·H₂O (9.24 mg, 0.386 mmol) in water (1 mL) to afford after workup and purification using method-M, 2-(2-((2-(3-aminocyclobutyl)-5-(1-aminoisoquinolin-7-yl)-2H-indazol-3-yl)methoxy)phenyl)acetic acid (138b) (27 mg, 42.5% yield) HCl salt as a yellow solid; ¹H NMR (300 MHz, DMSO-d₆) δ 13.85-13.35 (m, 1H, D₂O exchangeable), 9.91-9.17 (m, 1H, D₂O exchangeable), 9.16-9.07 (m, 1H), 8.63-8.54 (m, 3H, 2H D₂O exchangeable), 8.45 (d, J=8.5 Hz, 1H), 8.04 (d, J=8.5 Hz, 1H), 8.01-7.93 (m, 1H), 7.85 (t, J=8.1 Hz, 1H), 7.70 (t, J=5.6 Hz, 1H), 7.40-7.28 (m, 2H), 7.27-7.17 (m, 2H), 7.03-6.92 (m, 1H), 5.74-5.60 (m, 2H), 5.26-5.11 (m, 1H), 3.74-3.61 (m, 1H), 3.55-3.49 (m, 2H), 3.00-2.83 (m, 4H); MS (ES+): 494.2 (M+1); (ES−): 492.2 (M−1).

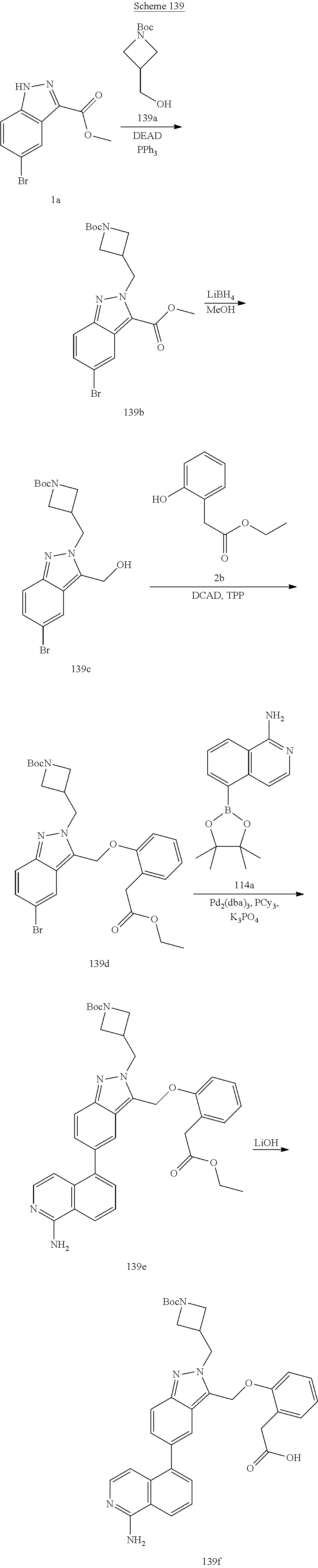

### Preparation of 2-(2-((5-(1-aminoisoquinolin-5-yl)-2-((1-(tert-butoxycarbonyl)azetidin-3-yl)methyl)-2H-indazol-3-yl)methoxy)phenyl)acetic acid (139f)

### Step-1: Preparation of methyl 5-bromo-2-((1-(tert-butoxycarbonyl)azetidin-3-yl)methyl)-2H-indazole-3-carboxylate (139b)

Compound 139b was prepared according to the procedure reported in step-1 of scheme-8 from methyl 5-bromo-1H-indazole-3-carboxylate (1a) (12 g, 47.0 mmol) in THF using DEAD (16.39 g, 94 mmol), PPh₃ (24.68 g, 94 mmol) and tert-butyl 3-(hydroxymethyl)azetidine-1-carboxylate (139a) (17.62 g, 94 mmol; CAS #142253-56-3) to afford after workup and purification using method-AR, methyl 5-bromo-2-((1-(tert-butoxycarbonyl)azetidin-3-yl)methyl)-2H-indazole-3-carboxylate (139b) (17 g, 85% yield) as yellow oil; 1H NMR (300 MHz, DMSO-d₆) δ 8.17 (d, J=1.9 Hz, 1H), 7.81 (d, J=9.0 Hz, 1H), 7.51 (dd, J=9.1, 1.8 Hz, 1H), 5.07 (d, J=7.4 Hz, 2H), 4.02 (s, 3H), 3.91 (t, J=8.3 Hz, 2H), 3.83-3.68 (m, 2H), 3.25-3.08 (m, 1H), 1.37 (s, 9H); MS (ES+): 424.1 and 426.1 (M+1).

### Step-2: Preparation of tert-butyl 3-((5-bromo-3-(hydroxymethyl)-2H-indazol-2-yl)methyl)azetidine-1-carboxylate (139c)

Compound 139c was prepared according to the procedure reported in step-2 of scheme-119 from methyl 5-bromo-2-((1-(tert-butoxycarbonyl)azetidin-3-yl)methyl)-2H-indazole-3-carboxylate (139b) (17 g, 40.1 mmol) in THF (100 mL) using LiBH₄ (4M in THF, 30.1 mL, 120 mmol) and MeOH (4.86 mL, 120 mmol) to afford after workup and purification using method-X, tert-butyl 3-((5-bromo-3-(hydroxymethyl)-2H-indazol-2-yl)methyl)azetidine-1-carboxylate (139c) (13.5 g, 85% yield) as a white solid; ¹H NMR (300 MHz, DMSO-d₆) & 8.03 (d, J=1.9 Hz, 1H), 7.55 (d, J=9.0 Hz, 1H), 7.30 (dd, J=9.1, 1.9 Hz, 1H), 5.52 (t, J=5.6 Hz, 1H), 4.89 (d, J=5.4 Hz, 2H), 4.65 (d, J=7.4 Hz, 2H), 3.96-3.84 (m, 2H), 3.84-3.67 (m, 2H), 3.25-3.07 (m, 1H), 1.37 (s, 9H); MS (ES+): 396.1 and 398.1 (M+1).

### Step-3: Preparation of tert-butyl 3-((5-bromo-3-((2-(2-ethoxy-2-oxoethyl)phenoxy)methyl)-2H-indazol-2-yl)methyl)azetidine-1-carboxylate (139d)

Compound 139d was prepared according to the procedure reported in step-2 of scheme-2, from tert-butyl 3-((5-bromo-3-(hydroxymethyl)-2H-indazol-2-yl)methyl)azetidine-1-carboxylate (139c) (10.5 g, 26.5 mmol) in DCM (100 mL) using PPh₃ (7.64 g, 29.1 mmol), ethyl 2-(2-hydroxyphenyl)acetate (2b) (5.25 g, 29.1 mmol) and a solution of DCAD (10.70 g, 29.1 mmol) in DCM (20 mL) to afford after workup and purification using method-K, tert-butyl 3-((5-bromo-3-((2-(2-ethoxy-2-oxoethyl)phenoxy)methyl)-2H-indazol-2-yl)methyl)azetidine-1-carboxylate (139d) (10 g, 67.6% yield) as yellow oil; MS (ES+): 558.2 and 560.1 (M+1); (ES−): 556.1 and 558.1 (M−1).

### Step-4: Preparation of tert-butyl 3-((5-(1-aminoisoquinolin-5-yl)-3-((2-(2-ethoxy-2-oxoethyl)phenoxy)methyl)-2H-indazol-2-yl)methyl)azetidine-1-carboxylate (139e)

Compound 139e was prepared according to the procedure reported in step-3 of scheme-112 from tert-butyl 3-((5-bromo-3-((2-(2-ethoxy-2-oxoethyl)phenoxy)methyl)-2H-indazol-2-yl)methyl)azetidine-1-carboxylate (139d) (250 mg, 0.448 mmol) in dioxane/2-MeTHF (4 mL, 3:1) using 5-(4,4,5,5-tetramethyl-1,3,2-dioxaborolan-2-yl)isoquinolin-1-amine (114a) (181 mg, 0.671 mmol), K₃PO₄ (2M aqueous solution, 0.336 mL, 1.343 mmol), PCy₃ (50.2 mg, 0.179 mmol) and Pd₂(dba)₃ (82 mg, 0.090 mmol) to afford after workup and purification using method-U, tert-butyl 3-((5-(1-aminoisoquinolin-5-yl)-3-((2-(2-ethoxy-2-oxoethyl)phenoxy)methyl)-2H-indazol-2-yl)methyl)azetidine-1-carboxylate (139e) (152 mg, 54.6% yield) as a clear oil; MS (ES+): 622.3 (M+1).

### Step-5: Preparation of 2-(2-((5-(1-aminoisoquinolin-5-yl)-2-((1-(tert-butoxycarbonyl)azetidin-3-yl)methyl)-2H-indazol-3-yl)methoxy)phenyl)acetic acid (139f)

Compound 139f was prepared according to the procedure reported step-2 of scheme-1 from tert-butyl 3-((5-(1-aminoisoquinolin-5-yl)-3-((2-(2-ethoxy-2-oxoethyl)phenoxy)methyl)-2H-indazol-2-yl)methyl)azetidine-1-carboxylate (139e) (76 mg, 0.122 mmol) in THF (3 mL) using a solution of LiOH·H₂O (32.2 mg, 1.343 mmol) in water (1 mL) to afford after workup and purification using method-M, 2-(2-((5-(1-aminoisoquinolin-5-yl)-2-((1-(tert-butoxycarbonyl)azetidin-3-yl)methyl)-2H-indazol-3-yl)methoxy)phenyl)acetic acid (1391) (44 mg, 33% yield) HCl salt as a white solid; 1H NMR (300 MHz, DMSO-d₆) δ 13.34 (s, 1H, D₂O exchangeable), 12.15 (s, 1H, D₂O exchangeable), 9.19 (s, 2H, D₂O exchangeable), 8.63 (d, J=8.3 Hz, 1H), 8.02-7.91 (m, 2H), 7.90-7.74 (m, 2H), 7.64 (d, J=7.2 Hz, 1H), 7.39-7.25 (m, 3H), 7.21 (d, J=7.4 Hz, 1H), 7.03-6.88 (m, 2H), 5.64 (s, 2H), 4.75 (d, J=7.5 Hz, 2H), 4.04-3.90 (m, 2H), 3.90-3.72 (m, 2H), 3.48 (s, 2H), 3.29-3.15 (m, 1H), 1.38 (s, 9H); MS (ES+): 594.3 (M+1); (ES−): 592.2 (M−1).

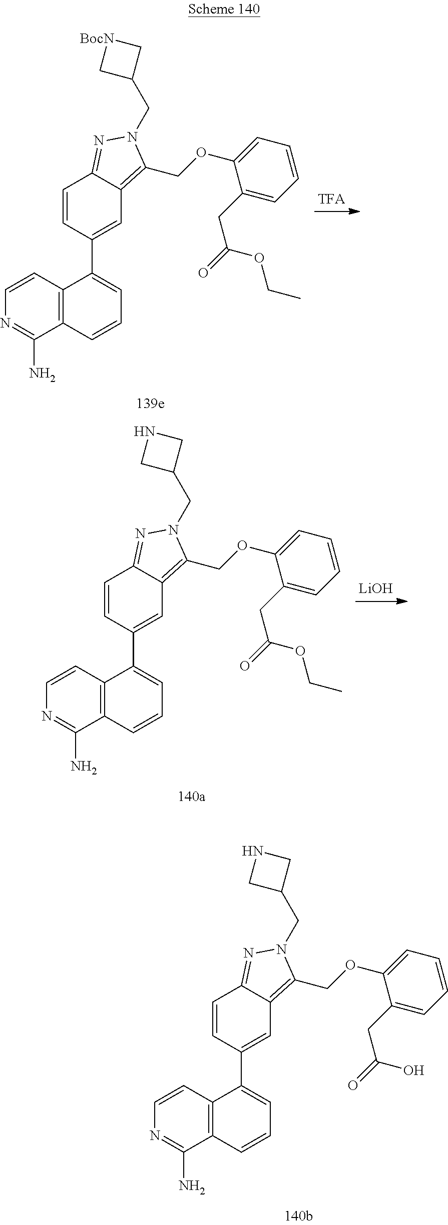

### Preparation of 2-(2-((5-(1-aminoisoquinolin-5-yl)-2-(azetidin-3-ylmethyl)-2H-indazol-3-yl)methoxy)phenyl)acetic acid (140b)

### Step-1: Preparation of ethyl 2-(2-((5-(1-aminoisoquinolin-5-yl)-2-(azetidin-3-ylmethyl)-2H-indazol-3-yl)methoxy)phenyl)acetate (140a)

Compound 140a was prepared according to the procedure reported in step-4 of scheme-9 from tert-butyl 3-((5-(1-aminoisoquinolin-5-yl)-3-((2-(2-ethoxy-2-oxoethyl)phenoxy)methyl)-2H-indazol-2-yl)methyl)azetidine-1-carboxylate (139e) (76 mg, 0.122 mmol) in DCM (4 mL) using TFA (0.094 mL, 1.222 mmol) to afford after workup ethyl 2-(2-((5-(1-aminoisoquinolin-5-yl)-2-(azetidin-3-ylmethyl)-2H-indazol-3-yl)methoxy)phenyl)acetate (140a) (50 mg, 78% yield) as a yellow oil; MS (ES+): 522.2 (M+1).

### Step-2: Preparation of 2-(2-((5-(1-aminoisoquinolin-5-yl)-2-(azetidin-3-ylmethyl)-2H-indazol-3-yl)methoxy)phenyl)acetic acid (140b)

Compound 140b was prepared according to the procedure reported step-2 of scheme-1 from ethyl 2-(2-((5-(1-aminoisoquinolin-5-yl)-2-(azetidin-3-ylmethyl)-2H-indazol-3-yl)methoxy)phenyl)acetate (140a) (50 mg, 0.096 mmol) in THF (3 mL) using a solution of LiOH·H₂O (8.78 mg, 0.367 mmol) in water (1 mL) to afford after workup and purification using method-M, 2-(2-((5-(1-aminoisoquinolin-5-yl)-2-(azetidin-3-ylmethyl)-2H-indazol-3-yl)methoxy)phenyl)acetic acid (140b) (28 mg, 46.4% yield) HCl salt as a yellow solid; ¹H NMR (300 MHz, DMSO-d₆) δ 13.66 (s, 1H, D₂O exchangeable), 9.63-9.10 (m, 4H, D₂O exchangeable), 8.70 (d, J=8.2 Hz, 1H), 8.00 (s, 1H), 7.95 (d, J=7.3 Hz, 1H), 7.85 (t, J=7.8 Hz, 1H), 7.79 (d, J=8.9 Hz, 1H), 7.67 (d, J=7.2 Hz, 1H), 7.38-7.26 (m, 3H), 7.22 (d, J=7.3 Hz, 1H), 7.01-6.91 (m, 2H), 5.66 (s, 2H), 4.85 (d, J=7.3 Hz, 2H), 4.03-3.96 (m, 4H), 3.50 (s, 2H), 3.46-3.38 (m, 1H); MS (ES+): 494.2 (M+1); (ES−): 492.1 (M−1).

### Scheme 141

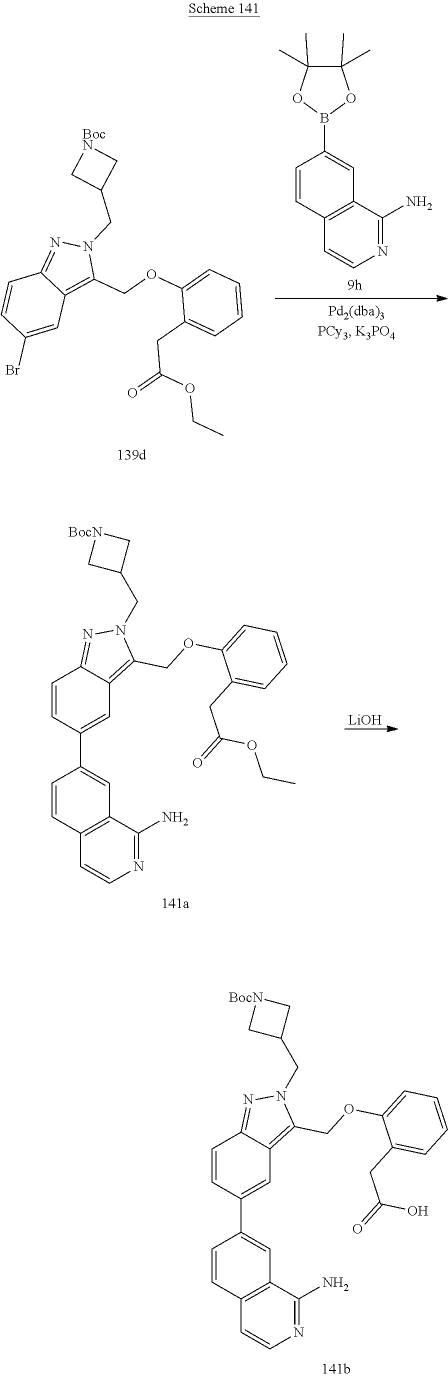 [SMILES: CCOC(=O)Cc1ccccc1OCc1c2cc(Br)ccc2nn1CC1CN(C(=O)OC(C)(C)C)C1 ; CC1(C)OB(c2ccc3ccnc(N)c3c2)OC1(C)C ; CCOC(=O)Cc1ccccc1OCc1c2cc(-c3ccc4ccnc(N)c4c3)ccc2nn1CC1CN(C(=O)OC(C)(C)C)C1 ; "LiOH" ; CC(C)(C)OC(=O)N1CC(Cn2nc3ccc(-c4ccc5ccnc(N)c5c4)cc3c2COc2ccccc2CC(=O)O)C1]

### Preparation of 2-(2-((5-(1-aminoisoquinolin-7-yl)-2-((1-(tert-butoxycarbonyl)azetidin-3-yl)methyl)-2H-indazol-3-yl)methoxy)phenyl)acetic acid (141b)

### Step-1: Preparation of tert-butyl 3-((5-(1-aminoisoquinolin-7-yl)-3-((2-(2-ethoxy-2-oxoethyl)phenoxy)methyl)-2H-indazol-2-yl)methyl)azetidine-1-carboxylate (141a)

Compound 141a was prepared according to the procedure reported in step-3 of scheme-112 from tert-butyl 3-((5-bromo-3-((2-(2-ethoxy-2-oxoethyl)phenoxy)methyl)-2H-indazol-2-yl)methyl)azetidine-1-carboxylate (139d) (250 mg, 0.448 mmol) in dioxane/2-Me THF (4 mL, 3:1) using 7-(4,4,5,5-tetramethyl-1,3,2-dioxaborolan-2-yl)isoquinolin-1-amine (9h) (181 mg, 0.671 mmol), K₃PO₄ (2M aqueous solution, 0.336 mL, 1.343 mmol), PCy₃ (37.7 mg, 0.134 mmol) and Pd₂(dba)₃ (82 mg, 0.090 mmol) to afford after workup and purification using method-U, tert-butyl 3-((5-(1-aminoisoquinolin-7-yl)-3-((2-(2-ethoxy-2-oxoethyl)phenoxy)methyl)-2H-indazol-2-yl)methyl)azetidine-1-carboxylate (141a) (188 mg, 67.5% yield) as a clear oil; MS (ES+): 622.3 (M+1).

### Step-2: Preparation of 2-(2-((5-(1-aminoisoquinolin-7-yl)-2-((1-(tert-butoxycarbonyl)azetidin-3-yl)methyl)-2H-indazol-3-yl)methoxy)phenyl)acetic acid (141b)

Compound 141b was prepared according to the procedure reported step-2 of scheme-1 from tert-butyl 3-((5-(1-aminoisoquinolin-7-yl)˜3-((2-(2-ethoxy-2-oxoethyl)phenoxy)methyl)-2H-indazol-2-yl)methyl)azetidine-1-carboxylate (141a) (188 mg, 0.302 mmol) in THF (3 mL) using a solution of LiOH·H₂O (32.2 mg, 1.343 mmol) in water (1 mL) to afford after workup and purification using method-M, 2-(2-((5-(1-aminoisoquinolin-7-yl)-2-((1-(tert-butoxycarbonyl)azetidin-3-yl)methyl)-2H-indazol-3-yl)methoxy)phenyl)acetic acid (141b) (75 mg, 28.2% yield) HCl salt as a white solid; 1H NMR (300 MHz, DMSO-d₆) δ 13.42 (s, 1H, D₂O exchangeable), 12.17 (s, 1H, D₂O exchangeable), 9.56-9.06 (m, 2H, D₂O exchangeable), 9.01 (s, 1H), 8.49 (s, 1H), 8.44 (dd, J=8.6, 1.7 Hz, 1H), 8.04 (d, J=8.5 Hz, 1H), 7.91 (dd, J=9.1, 1.8 Hz, 1H), 7.82 (d, J=9.0 Hz, 1H), 7.69 (d, J=6.9 Hz, 1H), 7.39-7.31 (m, 2H), 7.30-7.19 (m, 2H), 7.02-6.90 (m, 1H), 5.68 (s, 2H), 4.73 (d, J=7.5 Hz, 2H), 4.05-3.89 (m, 2H), 3.89-3.74 (m, 2H), 3.50 (s, 2H), 3.28-3.12 (m, 1H), 1.37 (s, 9H); MS (ES+): 594.3 (M+1); (ES−): 592.2 (M−1).

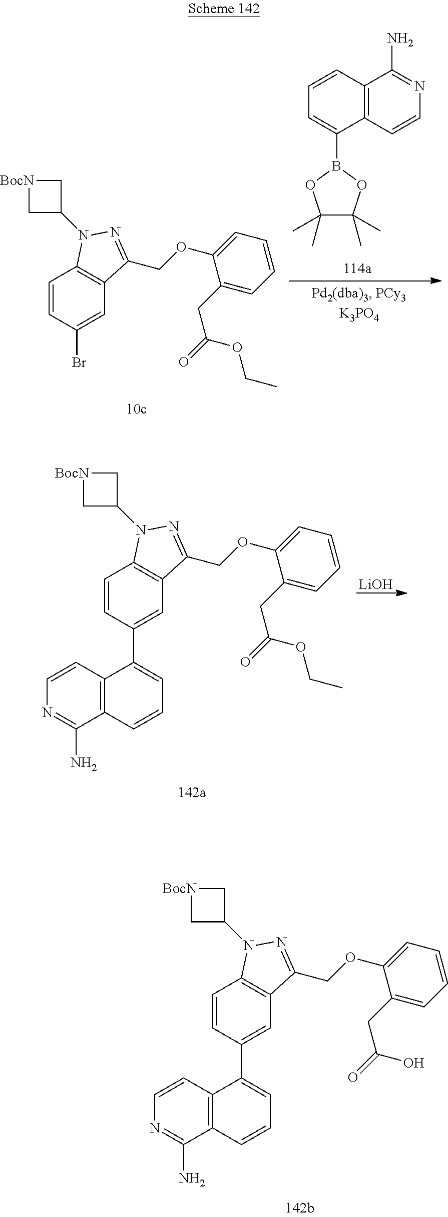

### Preparation of 2-(2-((5-(1-aminoisoquinolin-5-yl)-1-(1-(tert-butoxycarbonyl)azetidin-3-yl)-1H-indazol-3-yl)methoxy)phenyl)acetic acid (142b)

### Step-1: Preparation of tert-butyl 3-(5-(1-aminoisoquinolin-5-yl)-3-((2-(2-ethoxy-2-oxoethyl)phenoxy)methyl)-1H-indazol-1-yl)azetidine-1-carboxylate (142a)

Compound 142a was prepared according to the procedure reported in step-3 of scheme-112 from tert-butyl 3-(5-bromo-3-((2-(2-ethoxy-2-oxoethyl)phenoxy)methyl)-1H-indazol-1-yl)azetidine-1-carboxylate (10c) (250 mg, 0.459 mmol) in dioxane (4 mL) using 5-(4,4,5,5-tetramethyl-1,3,2-dioxaborolan-2-yl)isoquinolin-1-amine (114a) (186 mg, 0.689 mmol), K₃PO₄ (2M aqueous solution, 0.344 mL, 1.378 mmol), PCy₃ (51.5 mg, 0.184 mmol) and Pd₂(dba)₃ (84 mg, 0.092 mmol) to afford after workup and purification using method-U, tert-butyl 3-(5-(1-aminoisoquinolin-5-yl)˜3-((2-(2-ethoxy-2-oxoethyl)phenoxy)methyl)-1H-indazol-1-yl)azetidine-1-carboxylate (142a) (120 mg, 43.0% yield) as a clear oil; MS (ES+): 608.3 (M+1).

### Step-2: Preparation of 2-(2-((5-(1-aminoisoquinolin-5-yl)-1-(1-(tert-butoxycarbonyl)azetidin-3-yl)-1H-indazol-3-yl)methoxy)phenyl)acetic acid (142b)

Compound 142b was prepared according to the procedure reported step-2 of scheme-1 from tert-butyl 3-(5-(1-aminoisoquinolin-5-yl)-3-((2-(2-ethoxy-2-oxoethyl)phenoxy)methyl)-1H-indazol-1-yl)azetidine-1-carboxylate (142a) (120 mg, 0.197 mmol) in THF (3 mL) using a solution of LiOH·H₂O (33.0 mg, 1.378 mmol) in water (1 mL) to afford after workup and purification using method-M, 2-(2-((5-(1-aminoisoquinolin-5-yl)-1-(1-(tert-butoxycarbonyl)azetidin-3-yl)-1H-indazol-3-yl)methoxy)phenyl)acetic acid (142b) (38 mg, 14.28% yield) HCl salt as a white solid; H NMR (300 MHz, DMSO-d₆) δ 13.22 (s, 1H, D₂O exchangeable), 12.02 (s, 1H, D₂O exchangeable), 9.14 (s, 2H, D₂O exchangeable), 8.61 (d, J=8.3 Hz, 1H), 7.99-7.90 (m, 2H), 7.90-7.80 (m, 2H), 7.61 (d, J=7.3 Hz, 1H), 7.52 (d, J=8.7 Hz, 1H), 7.31-7.22 (m, 2H), 7.18 (d, J=7.4 Hz, 1H), 6.99-6.86 (m, 2H), 5.86-5.72 (m, 1H), 5.50 (s, 2H), 4.52-4.38 (m, 2H), 4.38-4.20 (m, 2H), 3.48 (s, 2H), 1.45 (s, 9H); MS (ES+): 580.2 (M+1); (ES−): 578.2 (M−1).

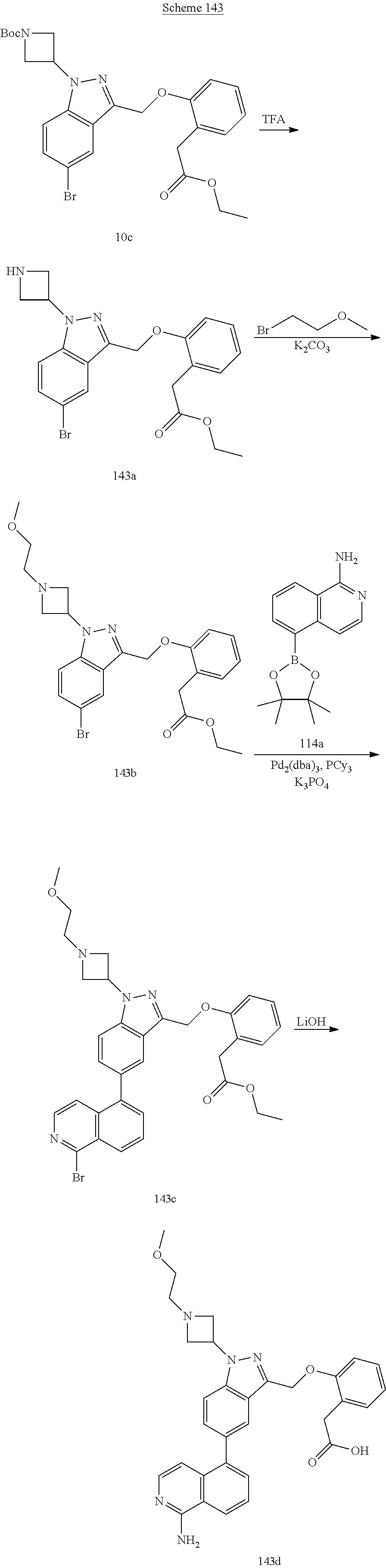

### Preparation of 2-(2-((5-(1-aminoisoquinolin-5-yl)-1-(1-(2-methoxyethyl)azetidin-3-yl)-1H-indazol-3-yl)methoxy)phenyl)acetic acid (143d)

### Step-1: Preparation of ethyl 2-(2-((1-(azetidin-3-yl)-5-bromo-1H-indazol-3-yl)methoxy)phenyl)acetate (143a)

Compound 143a was prepared according to the procedure reported in step-4 of scheme-9 from tert-butyl 3-(5-bromo-3-((2-(2-ethoxy-2-oxoethyl)phenoxy)methyl)-1H-indazol-1-yl)azetidine-1-carboxylate (10c) (1.8 g, 3.31 mmol) in DCM (100 mL) using TFA (2.55 mL, 33.1 mmol) to afford after workup and purification using method-AT, ethyl 2-(2-((1-(azetidin-3-yl)-5-bromo-1H-indazol-3-yl)methoxy)phenyl)acetate (143a) (1.3 g, 88% yield) as a white solid; 1H NMR (300 MHz, DMSO-d₆) δ 9.11 (s, 2H), 8.02 (s, 1H), 7.73 (d, J=8.9 Hz, 1H), 7.63 (d, J=9.0 Hz, 1H), 7.39-7.24 (m, 2H), 7.23 (d, J=7.4 Hz, 1H), 6.96 (t, J=6.8 Hz, 1H), 5.96-5.79 (m, 1H), 5.46 (s, 2H), 4.56-4.38 (m, 4H), 3.94 (q, J=7.1 Hz, 2H), 3.56 (s, 2H), 0.97 (t, J=7.1 Hz, 3H); ¹⁹F NMR (282 MHz, DMSO-d₆) δ −73.44; MS (ES+): 444.0 and 446.1 (M+1); (ES−): 442.0 and 444.0 (M−1).

### Step-2: Preparation of ethyl 2-(2-((5-bromo-1-(1-(2-methoxyethyl)azetidin-3-yl)-1H-indazol-3-yl)methoxy)phenyl)acetate (143b)

Compound 143b was prepared according to the procedure reported in step-3 of scheme-106 from ethyl 2-(2-((1-(azetidin-3-yl)-5-bromo-1H-indazol-3-yl)methoxy)phenyl)acetate (143a) (430 mg, 0.968 mmol) in ACN (10 mL) using 1-bromo-2-methoxyethane (202 mg, 1.452 mmol) and K₂CO₃ (669 mg, 4.84 mmol) to afford after workup and purification using method-P, ethyl 2-(2-((5-bromo-1-(1-(2-methoxyethyl)azetidin-3-yl)-1H-indazol-3-yl)methoxy)phenyl)acetate (143b) (280 mg, 57.6% yield) as yellow solid; MS (ES+): 502.1 and 504.1 (M+1).

### Step-3: Preparation of ethyl 2-(2-((5-(1-aminoisoquinolin-5-yl)-1-(1-(2-methoxyethyl)azetidin-3-yl)-1H-indazol-3-yl)methoxy)phenyl)acetate (143c)

Compound 143c was prepared according to the procedure reported in step-3 of scheme-112 from ethyl 2-(2-((5-bromo-1-(1-(2-methoxyethyl)azetidin-3-yl)-1H-indazol-3-yl)methoxy)phenyl)acetate (143b) (140 mg, 0.279 mmol) in dioxane/2-MeTHF (4 mL, 3:1) using 5-(4,4,5,5-tetramethyl-1,3,2-dioxaborolan-2-yl)isoquinolin-1-amine (114a) (113 mg, 0.418 mmol), K₃PO₄ (2M aqueous solution, 0.209 mL, 0.836 mmol), PCy₃ (31.3 mg, 0.111 mmol) and Pd₂(dba)₃ (51.0 mg, 0.056 mmol) to afford after workup and purification using method-U, ethyl 2-(2-((5-(1-aminoisoquinolin-5-yl)-1-(1-(2-methoxyethyl)azetidin-3-yl)-1H-indazol-3-yl)methoxy)phenyl)acetate (143c) (58 mg, 36.8% yield) as a clear oil; MS (ES+): 566.3 (M+1).

### Step-4: Preparation of 2-(2-((5-(1-aminoisoquinolin-5-yl)-1-(1-(2-methoxyethyl)azetidin-3-yl)-1H-indazol-3-yl)methoxy)phenyl)acetic acid (143d)

Compound 143d was prepared according to the procedure reported step-2 of scheme-1 from ethyl 2-(2-((5-(1-aminoisoquinolin-5-yl)-1-(1-(2-methoxyethyl)azetidin-3-yl)-1H-indazol-3-yl)methoxy)phenyl)acetate (143c) (58 mg, 0.103 mmol) in THF (3 mL) using a solution of LiOH·H₂O (20.02 mg, 0.836 mmol) in water (1 mL) to afford after workup and purification using method-M, 2-(2-((5-(1-aminoisoquinolin-5-yl)-1-(1-(2-methoxyethyl)azetidin-3-yl)-1H-indazol-3-yl)methoxy)phenyl)acetic acid (143d) (5 mg, 3.34% yield) HCl salt as a white solid; 1H NMR (300 MHz, DMSO-d₆) δ 13.66 (s, 1H, D₂O exchangeable), 12.07 (s, 1H, D₂O exchangeable), 11.18 (m, 1H, D₂O exchangeable), 9.37 (s, 2H, D₂O exchangeable), 8.70 (d, J=8.2 Hz, 1H), 8.04-7.93 (m, 2H), 7.93-7.83 (m, 2H), 7.71-7.63 (m, 1H), 7.60-7.51 (m, 1H), 7.32-7.23 (m, 2H), 7.23-7.15 (m, 1H), 7.02-6.86 (m, 2H), 5.98-5.76 (m, 1H), 5.58-5.45 (m, 2H), 4.88-4.70 (m, 2H), 4.63-4.38 (m, 2H), 3.75-3.67 (m, 2H), 3.67-3.57 (m, 2H), 3.51 (s, 2H), 3.33 (s, 3H), 3.28-3.16 (m, 2H); MS (ES+): 538.2 (M+1); (ES−): 536.1 (M−1).

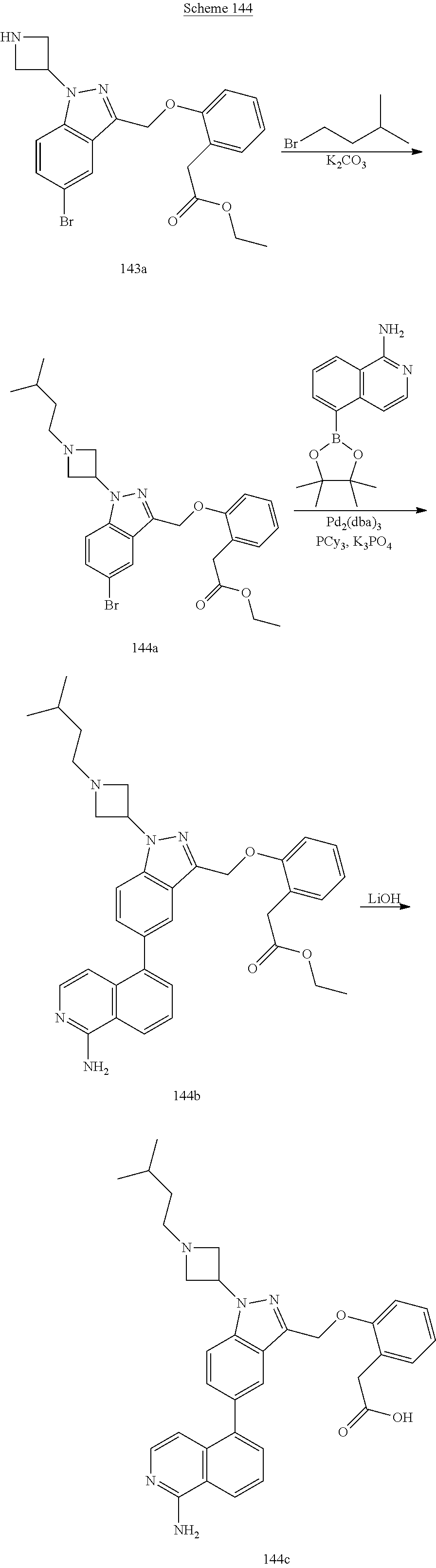

### Preparation of 2-(2-((5-(1-aminoisoquinolin-5-yl)-1-(1-isopentylazetidin-3-yl)-1H-indazol-3-yl)methoxy)phenyl)acetic acid (144c)

### Step-1: Preparation of ethyl 2-(2-((5-bromo-1-(1-isopentylazetidin-3-yl)-1H-indazol-3-yl)methoxy)phenyl)acetate (144a)

Compound 144a was prepared according to the procedure reported in step-3 of scheme-106 from ethyl 2-(2-((1-(azetidin-3-yl)-5-bromo-1H-indazol-3-yl)methoxy)phenyl)acetate (143a) (430 mg, 0.968 mmol) in ACN (10 mL) using 1-bromo-3-methylbutane (219 mg, 1.452 mmol) and K₂CO₃ (669 mg, 4.84 mmol) to afford after workup and purification using method-P, ethyl 2-(2-((5-bromo-1-(1-isopentylazetidin-3-yl)-1H-indazol-3-yl)methoxy)phenyl)acetate (144a) (346 mg, 69.5% yield) as yellow oil; MS (ES+): 514.1 and 516.2 (M+1).

### Step-2: Preparation of ethyl 2-(2-((5-(1-aminoisoquinolin-5-yl)-1-(1-isopentylazetidin-3-yl)-1H-indazol-3-yl)methoxy)phenyl)acetate (144b)

Compound 144b was prepared according to the procedure reported in step-3 of scheme-112 from ethyl 2-(2-((5-bromo-1-(1-isopentylazetidin-3-yl)-1H-indazol-3-yl)methoxy)phenyl)acetate (144a) (160 mg, 0.311 mmol) in dioxane/2-MeTHF (4 mL, 3:1) using 5-(4,4,5,5-tetramethyl-1,3,2-dioxaborolan-2-yl)isoquinolin-1-amine (114a) (126 mg, 0.467 mmol), K₃PO₄ (2M aqueous solution, 0.233 mL, 0.933 mmol), PCy₃ (34.9 mg, 0.124 mmol) and Pd₂(dba)₃ (57.0 mg, 0.062 mmol) to afford after workup and purification using method-U, ethyl 2-(2-((5-(1-aminoisoquinolin-5-yl)-1-(1-isopentylazetidin-3-yl)-1H-indazol-3-yl)methoxy)phenyl)acetate (144b) (63 mg, 35.1% yield) as a clear oil; MS (ES+): 578.3 (M+1).

### Step-3: Preparation of 2-(2-((5-(1-aminoisoquinolin-5-yl)-1-(1-isopentylazetidin-3-yl)-1H-indazol-3-yl)methoxy)phenyl)acetic acid (144c)

Compound 144c was prepared according to the procedure reported step-2 of scheme-1 from ethyl 2-(2-((5-(1-aminoisoquinolin-5-yl)-1-(1-isopentylazetidin-3-yl)-1H-indazol-3-yl)methoxy)phenyl)acetate (144b) (63 mg, 0.109 mmol) in THF (3 mL) using a solution of LiOH·H₂O (22.34 mg, 0.933 mmol) in water (1 mL) to afford after workup and purification using method-M, 2-(2-((5-(1-aminoisoquinolin-5-yl)-1-(1-isopentylazetidin-3-yl)-1H-indazol-3-yl)methoxy)phenyl)acetic acid (144c) (44 mg, 25.7% yield) HCl salt as a white solid; H NMR (300 MHz, DMSO-d₆) δ 13.62 (s, 1H, DO exchangeable), 12.05 (s, 1H, DO exchangeable), 9.34 (s, 2H, D₂O exchangeable), 8.70 (d, J=8.2 Hz, 1H), 7.98-7.81 (m, 4H), 7.67 (d, J=7.2 Hz, 1H), 7.56 (dd, J=8.7, 1.6 Hz, 1H), 7.35-7.24 (m, 2H), 7.24-7.16 (m, 1H), 6.98-6.86 (m, 2H), 6.12-5.74 (m, 1H), 5.54 (s, 2H), 4.88-4.22 (m, 4H), 3.51 (s, 2H), 3.45-3.40 (m, 2H), 1.77-1.62 (m, 1H), 1.59-1.47 (m, 2H), 0.94 (d, J=6.6 Hz, 6H); MS (ES+): 550.3 (M+1); (ES−): 548.2 (M−1).

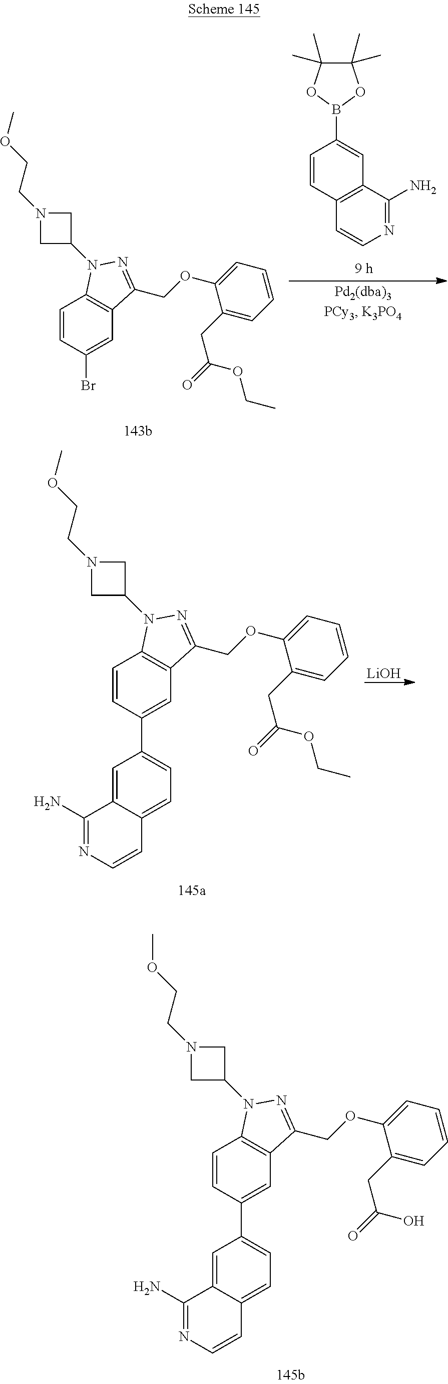

### Preparation of 2-(2-((5-(1-aminoisoquinolin-7-yl)-1-(1-(2-methoxyethyl)azetidin-3-yl)-1H-indazol-3-yl)methoxy)phenyl)acetic acid (145b)

### Step-1: Preparation of ethyl 2-(2-((5-(1-aminoisoquinolin-7-yl)-1-(1-(2-methoxyethyl)azetidin-3-yl)-1H-indazol-3-yl)methoxy)phenyl)acetate (145a)

Compound 145a was prepared according to the procedure reported in step-3 of scheme-112 from ethyl 2-(2-((5-bromo-1-(1-(2-methoxyethyl)azetidin-3-yl)-1H-indazol-3-yl)methoxy)phenyl)acetate (143b) (140 mg, 0.279 mmol) in dioxane (4 mL) using 7-(4,4,5,5-tetramethyl-1,3,2-dioxaborolan-2-yl)isoquinolin-I-amine (9h) (75 mg, 0.279 mmol), K₃PO₄ (2M aqueous solution, 0.209 mL, 0.836 mmol), PCy₃ (31.3 mg, 0.111 mmol) and Pd₂(dba)₃ (51.0 mg, 0.056 mmol) to afford after workup and purification using method-U, ethyl 2-(2-((5-(1-aminoisoquinolin-7-yl)-1-(1-(2-methoxyethyl)azetidin-3-yl)-1H-indazol-3-yl)methoxy)phenyl)acetate (145a) (52 mg, 33.0% yield) as a clear oil; MS (ES+): 566.2 (M+1).

### Step-2: Preparation of 2-(2-((5-(1-aminoisoquinolin-7-yl)-1-(1-(2-methoxyethyl)azetidin-3-yl)-1H-indazol-3-yl)methoxy)phenyl)acetic acid (145b)

Compound 145b was prepared according to the procedure reported step-2 of scheme-1 from ethyl 2-(2-((5-(1-aminoisoquinolin-7-yl)-1-(1-(2-methoxyethyl)azetidin-3-yl)-1H-indazol-3-yl)methoxy)phenyl)acetate (145a) (52 mg, 0.092 mmol) in THF (3 mL) using a solution of LiOH·H₂O (20.02 mg, 0.836 mmol) in water (1 mL) to afford after workup and purification using method-M, 2-(2-((5-(1-aminoisoquinolin-7-yl)-1-(1-(2-methoxyethyl)azetidin-3-yl)-1H-indazol-3-yl)methoxy)phenyl)acetic acid (145b) (15 mg, 10.01% yield) HCl salt as a white solid; ¹H NMR (300 MHz, DMSO-d₆) δ 13.44 (s, 1H, D₂O exchangeable), 12.11 (s, 1H, D₂O exchangeable), 9.30 (s, 2H, D₂O exchangeable), 9.04 (d, J=1.8 Hz, 1H), 8.48-8.39 (m, 2H), 8.15-8.03 (m, 2H), 7.95-7.87 (m, 1H), 7.71 (d, J=6.9 Hz, 1H), 7.34-7.26 (m, 3H), 7.25-7.19 (m, 1H), 7.00-6.90 (m, 1H), 6.15-5.75 (m, 1H), 5.59 (s, 2H), 4.88-4.34 (m, 4H), 3.71-3.65 (m, 2H), 3.65-3.59 (m, 2H), 3.56 (s, 2H), 3.32 (s, 3H); MS (ES+): 538.2 (M+1); (ES−): 536.2 (M−1).

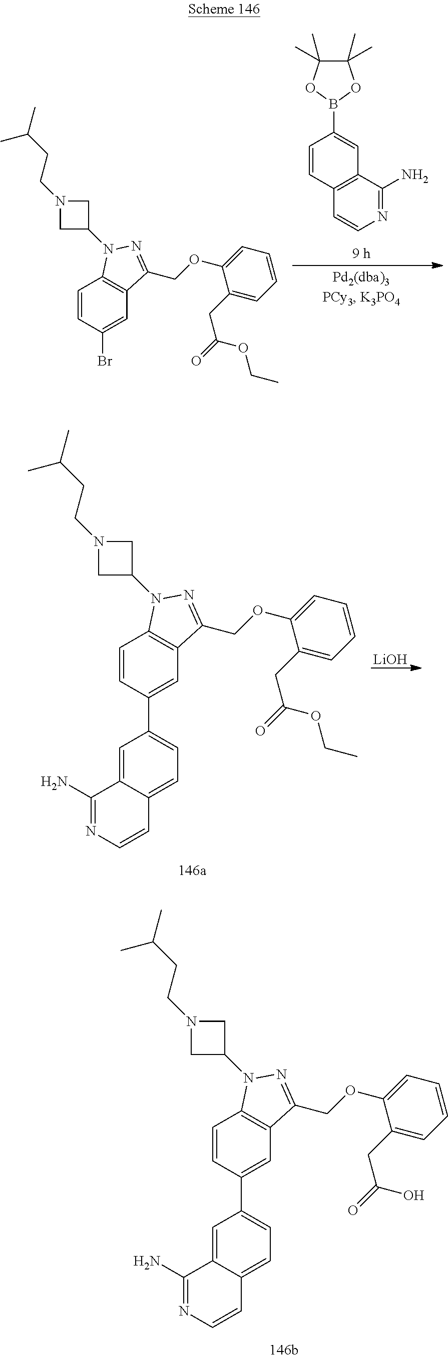

### Preparation of 2-(2-((5-(1-aminoisoquinolin-7-yl)-1-(1-isopentylazetidin-3-yl)-1H-indazol-3-yl)methoxy)phenyl)acetic acid (146b)

### Step-1: Preparation of ethyl 2-(2-((5-(1-aminoisoquinolin-7-yl)-1-(1-isopentylazetidin-3-yl)-1H-indazol-3-yl)methoxy)phenyl)acetate (146a)

Compound 146a was prepared according to the procedure reported in step-3 of scheme-112 from ethyl 2-(2-((5-bromo-1-(1-isopentylazetidin-3-yl)-1H-indazol-3-yl)methoxy)phenyl)acetate (144a) (160 mg, 0.311 mmol) in dioxane (4 mL) using 7-(4,4,5,5-tetramethyl-1,3,2-dioxaborolan-2-yl)isoquinolin-1-amine (9h) (84 mg, 0.311 mmol), K₃PO₄ (2M aqueous solution, 0.233 mL, 0.933 mmol), PCy₃ (34.9 mg, 0.124 mmol) and Pd₂(dba)₃ (57.0 mg, 0.062 mmol) to afford after workup and purification using method-U, ethyl 2-(2-((5-(1-aminoisoquinolin-7-yl)-1-(1-isopentylazetidin-3-yl)-1H-indazol-3-yl)methoxy)phenyl)acetate (146a) (63 mg, 35.1% yield) as a clear oil; MS (ES+): 578.3 (M+1).

### Step-2: Preparation of 2-(2-((5-(1-aminoisoquinolin-7-yl)-1-(1-isopentylazetidin-3-yl)-1H-indazol-3-yl)methoxy)phenyl)acetic acid (146b)

Compound 146b was prepared according to the procedure reported step-2 of scheme-1 from ethyl 2-(2-((5-(1-aminoisoquinolin-7-yl)-1-(1-isopentylazetidin-3-yl)-1H-indazol-3-yl)methoxy)phenyl)acetate (146a) (63 mg, 0.109 mmol) in THF (3 mL) using a solution of LiOH·H₂O (22.34 mg, 0.933 mmol) in water (1 mL) to afford after workup and purification using method-M, 2-(2-((5-(1-aminoisoquinolin-7-yl)-1-(1-isopentylazetidin-3-yl)-1H-indazol-3-yl)methoxy)phenyl)acetic acid (146b) (40 mg, 23.40% yield) HCl salt as a white solid; 1H NMR (300 MHz, DMSO-d₆) δ 13.44 (s, 1H, D₂O exchangeable), 12.12 (s, 1H, D₂O exchangeable), 11.58-10.58 (m, 1H, D₂O) exchangeable), 9.28 (s, 1H, D₂O exchangeable), 9.04 (s, 1H), 8.48-8.37 (m, 2H), 8.17-8.01 (m, 2H), 7.97-7.86 (m, 1H), 7.74-7.67 (m, 1H), 7.38-7.17 (m, 4H), 7.00-6.90 (m, 1H), 6.17-5.76 (m, 1H), 5.67-5.47 (m, 2H), 4.88-4.26 (m, 4H), 3.67-3.51 (m, 2H), 3.46-3.38 (m, 2H), 1.78-1.61 (m, 1H), 1.61-1.41 (m, 2H), 1.07-0.88 (m, 6H); MS (ES+): 550.3 (M+1); (ES−): 548.2 (M−1).

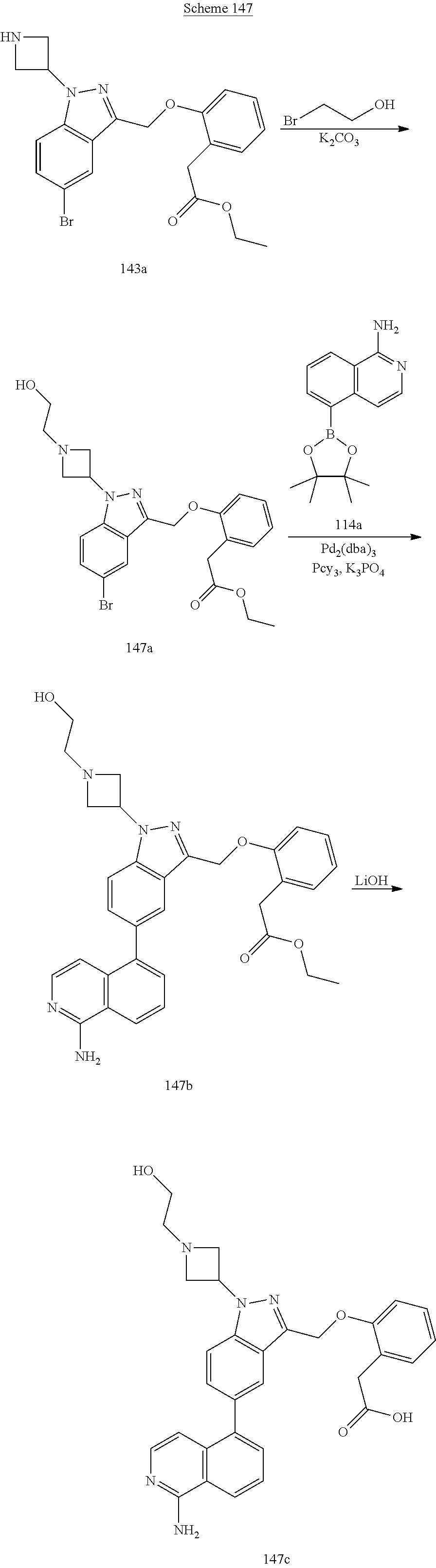

### Preparation of 2-(2-((5-(1-aminoisoquinolin-5-yl)-1-(1-(2-hydroxyethyl)azetidin-3-yl)-1H-indazol-3-yl)methoxy)phenyl)acetic acid (147c)

### Step-1: Preparation of ethyl 2-(2-((5-bromo-1-(1-(2-hydroxyethyl)azetidin-3-yl)-1H-indazol-3-yl)methoxy)phenyl)acetate (147a)

Compound 147a was prepared according to the procedure reported in step-3 of scheme-106 from ethyl 2-(2-((1-(azetidin-3-yl)-5-bromo-1H-indazol-3-yl)methoxy)phenyl)acetate (143a) (430 mg, 0.968 mmol) in ACN (10 mL) using 2-bromoethanol (181 mg, 1.452 mmol) and K₂CO₃ (669 mg, 4.84 mmol) to afford after workup and purification using method-P, ethyl 2-(2-((5-bromo-1-(1-(2-hydroxyethyl)azetidin-3-yl)-1H-indazol-3-yl)methoxy)phenyl)acetate (147a) (350 mg, 74.1% yield) as yellow solid; MS (ES+): 488.1 and 490.1 (M+1).

### Step-2: Preparation of ethyl 2-(2-((5-(1-aminoisoquinolin-5-yl)-1-(1-(2-hydroxyethyl)azetidin-3-yl)-1H-indazol-3-yl)methoxy)phenyl)acetate (147b)

Compound 147b was prepared according to the procedure reported in step-3 of scheme-112 from ethyl 2-(2-((5-bromo-1-(1-(2-hydroxyethyl)azetidin-3-yl)-1H-indazol-3-yl)methoxy)phenyl)acetate (147a) (160 mg, 0.328 mmol) in dioxane/2-MeTHF (4 mL, 3:1) using 5-(4,4,5,5-tetramethyl-1,3,2-dioxaborolan-2-yl)isoquinolin-1-amine (114a) (133 mg, 0.491 mmol), K₃PO₄ (2M aqueous solution, 0.246 mL, 0.983 mmol), PCy₃ (36.7 mg, 0.131 mmol) and Pd₂(dba)₃ (60.0 mg, 0.066 mmol) to afford after workup and purification using method-U, ethyl 2-(2-((5-(1-aminoisoquinolin-5-yl)-1-(1-(2-hydroxyethyl)azetidin-3-yl)-1H-indazol-3-yl)methoxy)phenyl)acetate (147b) (68 mg, 37.6% yield) as a clear oil; MS (ES+): 552.2 (M+1).

### Step-3: Preparation of 2-(2-((5-(1-aminoisoquinolin-5-yl)-1-(1-(2-hydroxyethyl)azetidin-3-yl)-1H-indazol-3-yl)methoxy)phenyl)acetic acid (147c)

Compound 147c was prepared according to the procedure reported step-2 of scheme-1 from ethyl 2-(2-((5-(1-aminoisoquinolin-5-yl)-1-(1-(2-hydroxyethyl)azetidin-3-yl)-1H-indazol-3-yl)methoxy)phenyl)acetate (147b) (68 mg, 0.123 mmol) in THF (3 mL) using a solution of LiOH·H₂O) (23.54 mg, 0.983 mmol) in water (1 mL) to afford after workup and purification using method-M, 2-(2-((5-(1-aminoisoquinolin-5-yl)-1-(1-(2-hydroxyethyl)azetidin-3-yl)-1H-indazol-3-yl)methoxy)phenyl)acetic acid (147c) (46 mg, 26.8% yield) HCl salt as a white solid; 1H NMR (300 MHz, DMSO-d₆) δ 13.53 (s, 1H), 12.05 (s, 1H), 11.18-10.46 (m, 1H), 9.30 (s, 2H), 8.75-8.61 (m, 1H), 8.01-7.81 (m, 4H), 7.73-7.61 (m, 1H), 7.61-7.50 (m, 1H), 7.34-7.24 (m, 2H), 7.24-7.15 (m, 1H), 6.99-6.86 (m, 2H), 6.15-5.77 (m, 1H), 5.66-5.45 (m, 2H), 5.45-5.17 (m, 1H), 4.96-4.29 (m, 4H), 3.82-3.67 (m, 2H), 3.60-3.46 (m, 4H); MS (ES+): 524.2 (M+1); (ES−): 522.1 (M−1).

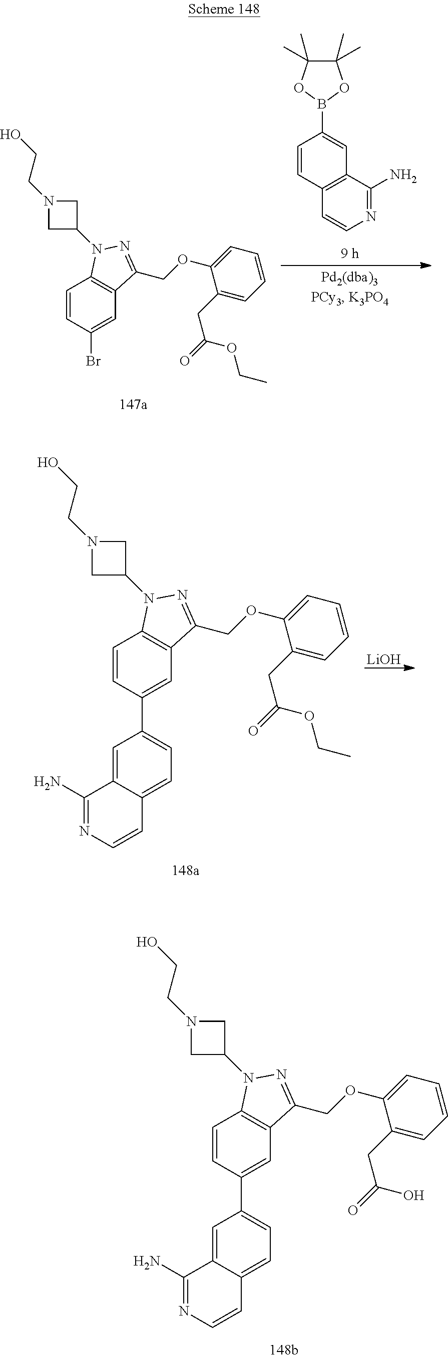

### Preparation of 2-(2-((5-(1-aminoisoquinolin-7-yl)-1-(1-(2-hydroxyethyl)azetidin-3-yl)-1H-indazol-3-yl)methoxy)phenyl)acetic acid (148b)

### Step-1: Preparation of ethyl 2-(2-((5-(1-aminoisoquinolin-7-yl)-1-(1-(2-hydroxyethyl)azetidin-3-yl)-1H-indazol-3-yl)methoxy)phenyl)acetate (148a)

Compound 148a was prepared according to the procedure reported in step-3 of scheme-112 from ethyl 2-(2-((5-bromo-1-(1-(2-hydroxyethyl)azetidin-3-yl)-1H-indazol-3-yl)methoxy)phenyl)acetate (147a) (160 mg, 0.328 mmol) in dioxane (4 mL) using 7-(4,4,5,5-tetramethyl-1,3,2-dioxaborolan-2-yl)isoquinolin-1-amine (9h) (89 mg, 0.328 mmol), K₃PO₄ (2M aqueous solution, 0.246 mL, 0.983 mmol), PCy₃ (36.7 mg, 0.131 mmol) and Pd₂(dba)₃ (60.0 mg, 0.066 mmol) to afford after workup and purification using method-U, ethyl 2-(2-((5-(1-aminoisoquinolin-7-yl)-1-(1-(2-hydroxyethyl)azetidin-3-yl)-1H-indazol-3-yl)methoxy)phenyl)acetate (148a) (54 mg, 29.9% yield) as a clear oil; MS (ES+): 552.3 (M+1).

### Step-2: Preparation of 2-(2-((5-(1-aminoisoquinolin-7-yl)-1-(1-(2-hydroxyethyl)azetidin-3-yl)-1H-indazol-3-yl)methoxy)phenyl)acetic acid (148b)

Compound 148b was prepared according to the procedure reported step-2 of scheme-1 from ethyl 2-(2-((5-(1-aminoisoquinolin-7-yl)-1-(1-(2-hydroxyethyl)azetidin-3-yl)-1H-indazol-3-yl)methoxy)phenyl)acetate (148a) (54 mg, 0.098 mmol) in THF (3 mL) using a solution of LiOH·H₂O (23.54 mg, 0.983 mmol) in water (1 mL) to afford after workup and purification using method-M, 2-(2-((5-(1-aminoisoquinolin-7-yl)-1-(1-(2-hydroxyethyl)azetidin-3-yl)-1H-indazol-3-yl)methoxy)phenyl)acetic acid (148b) (23 mg, 13.41% yield) HCl salt as a white solid; 1H NMR (300 MHz, DMSO-d₆) δ 13.43 (s, 1H, D₂O exchangeable), 12.14 (s, 1H, D₂O exchangeable), 9.31 (s, 2H, D₂O exchangeable), 9.05 (d, J=1.7 Hz, 1H), 8.49-8.40 (m, 2H), 8.19-8.09 (m, 1H), 8.05 (d, J=8.6 Hz, 1H), 7.91 (d, J=8.9 Hz, 1H), 7.71 (d, J=6.9 Hz, 1H), 7.39-7.18 (m, 4H), 6.95 (td, J=7.1, 1.7 Hz, 1H), 6.10-5.75 (m, 1H), 5.58 (s, 2H), 5.43-5.26 (m, 1H, D₂O exchangeable), 4.91-4.41 (m, 4H), 3.81-3.64 (m, 2H), 3.56 (s, 2H), 3.54-3.46 (m, 2H); MS (ES+): 524.2 (M+1); (ES−): 522.2 (M−1).

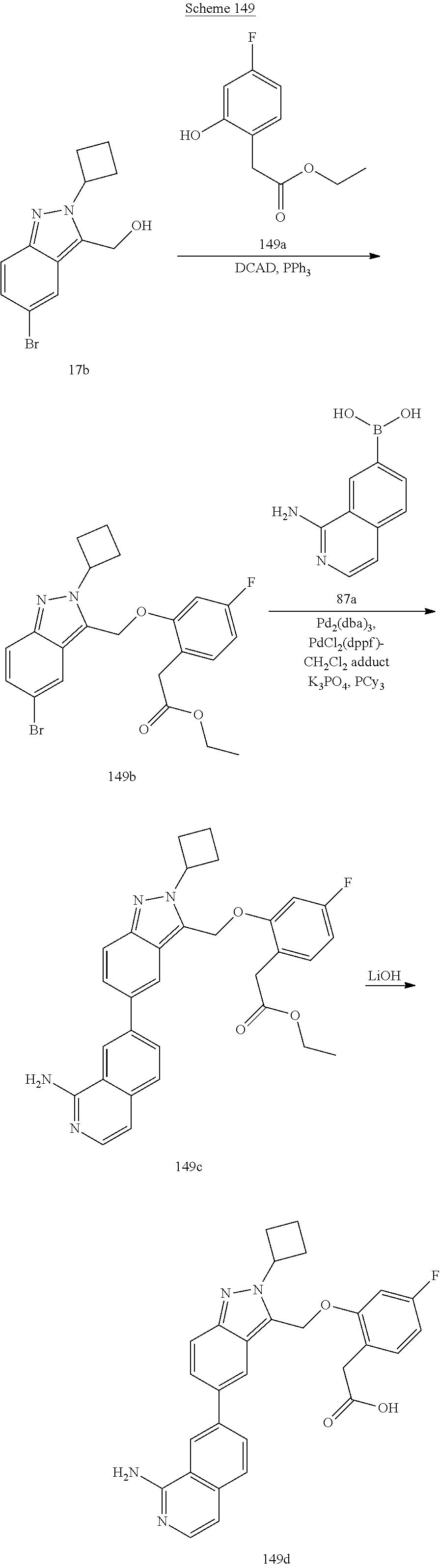

### Preparation of 2-(2-((5-(1-aminoisoquinolin-7-yl)-2-cyclobutyl-2H-indazol-3-yl)methoxy)-4-fluorophenyl)acetic acid (149d)

### Step-1: Preparation of ethyl 2-(2-((5-bromo-2-cyclobutyl-2H-indazol-3-yl)methoxy)-4-fluorophenyl)acetate (149b)

Compound 149b was prepared according to the procedure reported in step-2 of scheme-2, from (5-bromo-2-cyclobutyl-2H-indazol-3-yl)methanol (17b) (650 mg, 2.312 mmol) in DCM, using PPh₃ (667 mg, 2.54 mmol), ethyl 2-(4-fluoro-2-hydroxyphenyl)acetate (149a) (550 mg, 2.77 mmol; CAS #1261751-44-3) and DCAD (934 mg, 2.54 mmol) in DCM (15 mL) to afford after workup and purification using method-AU, ethyl 2-(2-((5-bromo-2-cyclobutyl-2H-indazol-3-yl)methoxy)-4-fluorophenyl)acetate (149b) (681 mg, 63.9% yield) as a white solid; MS (ES+): 461.1 (M+1).

### Step-2: Preparation of ethyl 2-(2-((5-(1-aminoisoquinolin-7-yl)-2-cyclobutyl-2H-indazol-3-yl)methoxy)-4-fluorophenyl)acetate (149c)

Compound 149c was prepared according to the procedure reported in step-5 of scheme-1 from ethyl 2-(2-((5-bromo-2-cyclobutyl-2H-indazol-3-yl)methoxy)-4-fluorophenyl)acetate (149b) (340 mg, 0.737 mmol) in dioxane/THF (12 mL, 1:1) using 1-aminoisoquinolin-7-ylboronic acid (87a) (180 mg, 0.958 mmol), K₃PO₄ (2M aqueous solution, 0.737 mL, 2.95 mmol), PCy₃ (41.3 mg, 0.147 mmol), Pd₂(dba)₃ (67.5 mg, 0.074 mmol) and PdCl₂(dppf)-CH₂Cl₂ adduct (60.2 mg, 0.074 mmol) to afford after workup and purification using method-AE, ethyl 2-(2-((5-(1-aminoisoquinolin-7-yl)-2-cyclobutyl-2H-indazol-3-yl)methoxy)-4-fluorophenyl)acetate (149c) (150 mg, 38.8% yield) as a yellow solid; MS (ES+): 525.2 (M+1).

### Step-3: Preparation of 2-(2-((5-(1-aminoisoquinolin-7-yl)-2-cyclobutyl-2H-indazol-3-yl)methoxy)-4-fluorophenyl)acetic acid (149d)

Compound 149d was prepared according to the procedure reported step-2 of scheme-1 from ethyl 2-(2-((5-(1-aminoisoquinolin-7-yl)-2-cyclobutyl-2H-indazol-3-yl)methoxy)-4-fluorophenyl)acetate (149c) in THF (3 mL) using a solution of LiOH·H₂O (36.0 mg, 0.858 mmol) in water (1 mL) to afford after workup and purification using method-M, 2-(2-((5-(1-aminoisoquinolin-7-yl)-2-cyclobutyl-2H-indazol-3-yl)methoxy)-4-fluorophenyl)acetic acid (149d) (25.7 mg, 18% yield) HCl salt as a white solid; 1H NMR (300 MHz, DMSO-d₆) δ 13.14 (s, 1H, D₂O exchangeable), 12.20 (s, 1H, D₂O exchangeable), 9.12 (s, 1H, D₂O exchangeable), 8.97 (s, 1H), 8.50-8.39 (m, 2H), 8.06 (d, J=8.5 Hz, 1H), 7.89 (s, 2H), 7.68 (d, J=7.0 Hz, 1H), 7.35-7.21 (m, 3H), 6.81 (t, J=8.6 Hz, 1H), 5.66 (s, 2H), 5.38-5.26 (m, 1H), 3.47 (s, 2H), 2.87-2.56 (m, 4H), 1.98-1.82 (m, 2H); ¹⁹F NMR (282 MHz, DMSO-d₆) δ −112.94; MS (ES+): 497.2 (M+1).

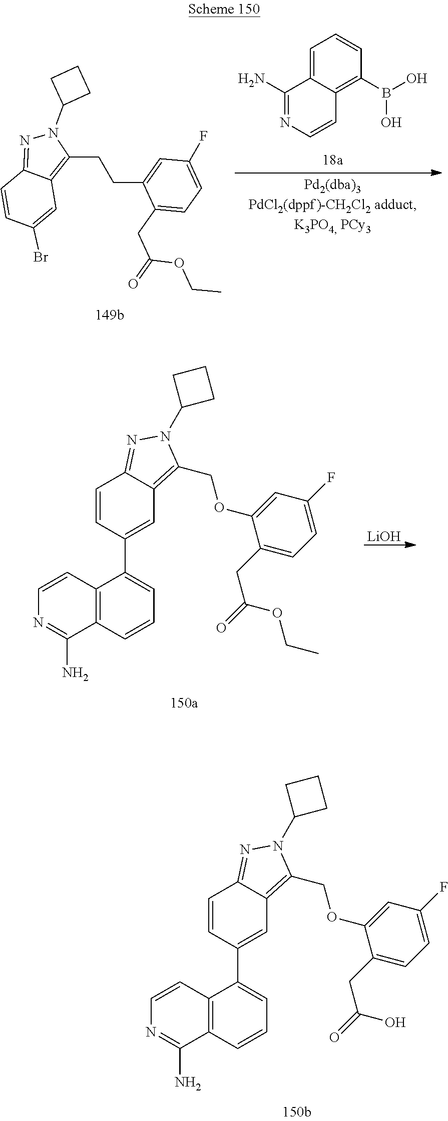

### Preparation of 2-(2-((5-(1-aminoisoquinolin-5-yl)-2-cyclobutyl-2H-indazol-3-yl)methoxy)-4-fluorophenyl)acetic acid (150b)

### Step-1: Preparation of ethyl 2-(2-((5-(1-aminoisoquinolin-5-yl)-2-cyclobutyl-2H-indazol-3-yl)methoxy)-4-fluorophenyl)acetate (150a)

Compound 150a was prepared according to the procedure reported in step-5 of scheme-1 from ethyl 2-(2-((5-bromo-2-cyclobutyl-2H-indazol-3-yl)methoxy)-4-fluorophenyl)acetate (149b) (340 mg, 0.737 mmol) in dioxane/THF (8 mL, 1:1) using (1-aminoisoquinolin-5-yl)boronic acid (18a) (139 mg, 0.737 mmol), K₃PO₄ (2M aqueous solution, 0.737 mL, 2.95 mmol), PCy₃ (41.3 mg, 0.147 mmol), Pd₂(dba)₃ (67.5 mg, 0.074 mmol) and PdCl₂(dppf)-CH₂Cl₂ adduct (60.2 mg, 0.074 mmol) to afford after workup and purification using method-AE, ethyl 2-(2-((5-(1-aminoisoquinolin-5-yl)-2-cyclobutyl-2H-indazol-3-yl)methoxy)-4-fluorophenyl)acetate (150a) (142 mg, 36.7% yield) as a yellow solid; MS (ES+): 525.2 (M+1).

### Step-2: Preparation of 2-(2-((5-(1-aminoisoquinolin-5-yl)-2-cyclobutyl-2H-indazol-3-yl)methoxy)-4-fluorophenyl)acetic acid (150b)

Compound 150b was prepared according to the procedure reported step-2 of scheme-1 from ethyl 2-(2-((5-(1-aminoisoquinolin-5-yl)-2-cyclobutyl-2H-indazol-3-yl)methoxy)-4-fluorophenyl)acetate (150a) in THF (3 mL) using a solution of LiOH·H₂O (34.1 mg, 0.812 mmol) in water (1 mL) to afford after workup and purification using method-M, 2-(2-((5-(1-aminoisoquinolin-5-yl)-2-cyclobutyl-2H-indazol-3-yl)methoxy)-4-fluorophenyl)acetic acid (150b) (32.6 mg, 24% yield) HCl salt as a white solid; ¹H NMR (300 MHz, DMSO-d₆) δ 13.25 (s, 1H, D₂O exchangeable), 12.21 (s, 1H, D₂O exchangeable), 9.19 (s, 2H, D₂O exchangeable), 8.63 (d, J=8.3 Hz, 1H), 8.04-7.93 (m, 2H), 7.93-7.80 (m, 2H), 7.64 (d, J=7.3 Hz, 1H), 7.34 (d, J=8.9 Hz, 1H), 7.29-7.16 (m, 2H), 7.00 (d, J=7.2 Hz, 1H), 6.79 (t, J=8.5 Hz, 1H), 5.63 (s, 2H), 5.42-5.25 (m, 1H), 3.47 (s, 2H), 2.87-2.55 (m, 4H), 2.00-1.83 (m, 2H); ¹⁹F NMR (282 MHz, DMSO-d₆) δ −113.05; MS (ES+) 497.2 (M+1).

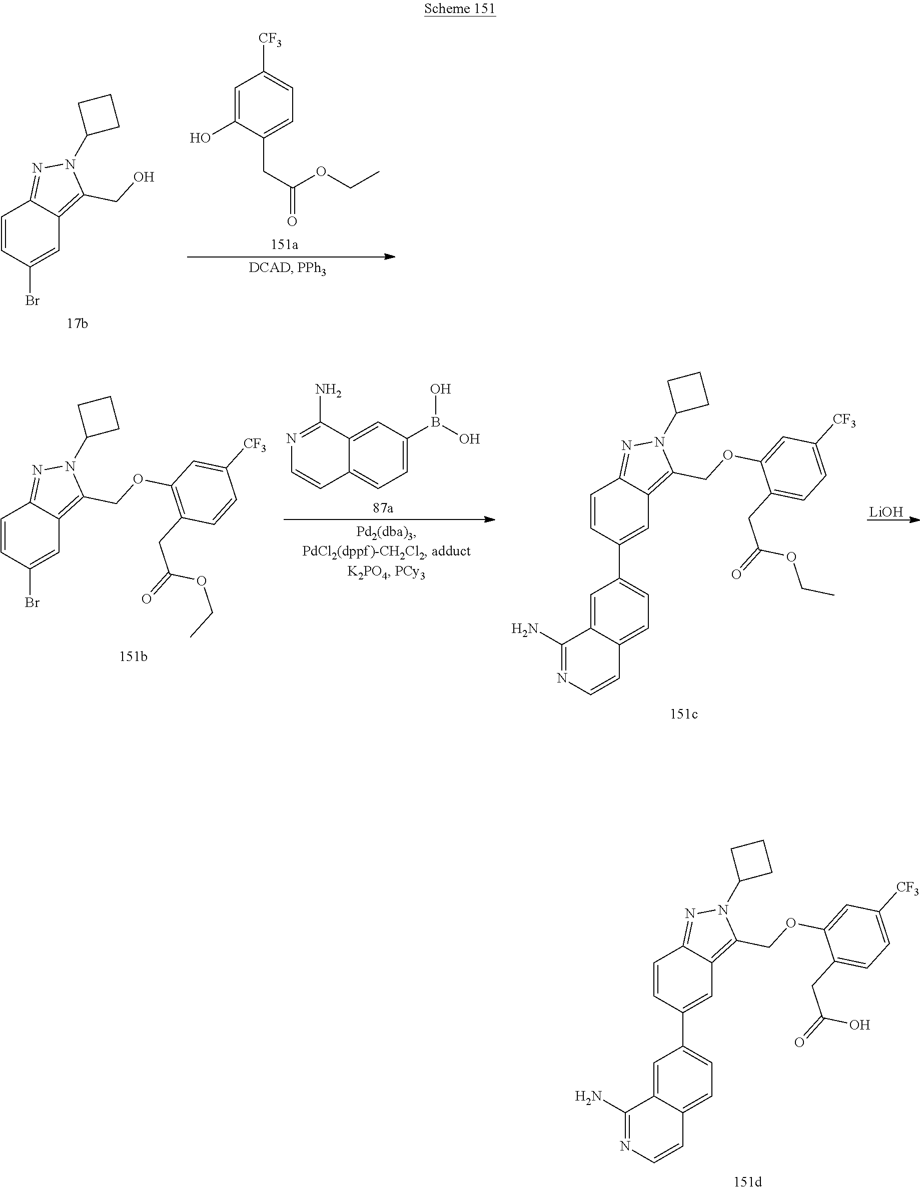

### Preparation of 2-(2-((5-(1-aminoisoquinolin-7-yl)-2-cyclobutyl-2H-indazol-3-yl)methoxy)-4-(trifluoromethyl)phenyl)acetic acid (151d)

### Step-1: Preparation of ethyl 2-(2-((5-bromo-2-cyclobutyl-2H-indazol-3-yl)methoxy)-4-(trifluoromethyl)phenyl)acetate (151b)

Compound 151b was prepared according to the procedure reported in step-2 of scheme 2, from (5-bromo-2-cyclobutyl-2H-indazol-3-yl)methanol (17b) (650 mg, 2.312 mmol) in DCM (20 mL) using PPh₃ (667 mg, 2.54 mmol), ethyl 2-(2-hydroxy-4-(trifluoromethyl)phenyl)acetate (151a) (689 mg, 2.77 mmol) and a solution of DCAD (934 mg, 2.54 mmol) in DCM to afford after workup and purification using method-AU, ethyl 2-(2-((5-bromo-2-cyclobutyl-2H-indazol-3-yl)methoxy)-4-(trifluoromethyl)phenyl)acetate (151b) (576 mg, 1.126 mmol, 48.7% yield) as a white solid; MS (ES+): 511.0 (M+1).

### Step-2: Preparation of ethyl 2-(2-((5-(1-aminoisoquinolin-7-yl)-2-cyclobutyl-2H-indazol-3-yl)methoxy)-4-(trifluoromethyl)phenyl)acetate (151c)

Compound 151c was prepared according to the procedure reported in step-5 of scheme 1 from ethyl 2-(2-((5-bromo-2-cyclobutyl-2H-indazol-3-yl)methoxy)-4-(trifluoromethyl)phenyl)acetate (151b) (235 mg, 0.460 mmol) in dioxane/THF (12 mL, 1:1) using 1-aminoisoquinolin-7-ylboronic acid (87a) (112 mg, 0.597 mmol), K₃PO₄ (2M aqueous solution, 0.460 mL, 1.838 mmol), PCy₃ (25.8 mg, 0.092 mmol), Pd₂(dba)₃ (42.1 mg, 0.046 mmol) and PdCl₂(dppf)-CH₂Cl₂ adduct (37.5 mg, 0.046 mmol) to afford after workup and purification using method-AE, ethyl 2-(2-((5-(1-aminoisoquinolin-7-yl)-2-cyclobutyl-2H-indazol-3-yl)methoxy)-4-(trifluoromethyl)phenyl)acetate (151c) (62 mg, 23.48% yield) as a yellow solid; MS (ES+): 575.2 (M+1).

### Step-3: Preparation of 2-(2-(5-(1-aminoisoquinolin-7-yl)-2-cyclobutyl-2H-indazol-3-yl)methoxy)-4-(trifluoromethyl)phenyl)acetic acid (151d)

Compound 151d was prepared according to the procedure reported step-2 of scheme 1 from ethyl 2-(2-((5-(1-aminoisoquinolin-7-yl)-2-cyclobutyl-2H-indazol-3-yl)methoxy)-4-(trifluoromethyl)phenyl)acetate (151c) (62 mg, 0.108 mmol) in THF (3 mL) using a solution of LiOH·H₂O (13.58 mg, 0.324 mmol) in water (1 mL) to afford after workup and purification using method-M, 2-(2-((5-(1-aminoisoquinolin-7-yl)-2-cyclobutyl-2H-indazol-3-yl)methoxy)-4-(trifluoromethyl)phenyl)acetic acid (151d) (9.7 mg, 16% yield) HCl salt as a white solid; ¹H NMR (300 MHz, DMSO-d₆) δ 13.06 (s, 1H, D₂O exchangeable), 12.32 (s, 1H, D₂O exchangeable), 9.12 (s, 1H, D₂O exchangeable), 8.95 (s, 1H), 8.49 (s, 1H), 8.44 (d, J=8.5 Hz, 1H), 8.06 (d, J=8.6 Hz, 1H), 7.94-7.85 (m, 2H), 7.72-7.63 (m, 2H), 7.47 (d, J=7.9 Hz, 1H), 7.34 (d, J=7.9 Hz, 1H), 7.28 (d, J=6.9 Hz, 1H), 5.78 (s, 2H), 5.40-5.25 (m, 1H), 3.59 (s, 2H), 2.85-2.67 (m, 4H), 1.98-1.79 (m, 2H); ¹⁹F NMR (282 MHz, DMSO-d₆) δ −62.04; MS (ES+): 547.2 (M+1).

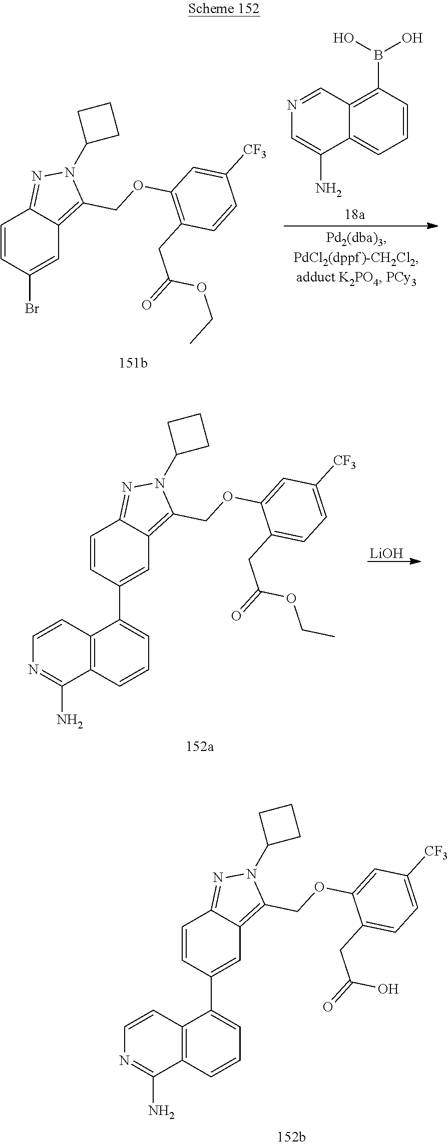

### Preparation of 2-(2-((5-(1-aminoisoquinolin-5-yl)-2-cyclobutyl-2H-indazol-3-yl)methoxy)-4-(trifluoromethyl)phenyl)acetic acid (152b)

### Step-1: Preparation of ethyl 2-(2-((5-(1-aminoisoquinolin-5-yl)-2-cyclobutyl-2H-indazol-3-yl)methoxy)-4-(trifluoromethyl)phenyl)acetate (152a)

Compound 152a was prepared according to the procedure reported in step-5 of scheme 1 from ethyl 2-(2-((5-bromo-2-cyclobutyl-2H-indazol-3-yl)methoxy)-4-(trifluoromethyl)phenyl)acetate (151b) (235 mg, 0.460 mmol) in dioxane/THF (8 mL, 1:1) using (1-aminoisoquinolin-5-yl)boronic acid (18a) (86 mg, 0.460 mmol), K₃PO₄ (2M aqueous solution, 0.460 mL, 1.838 mmol), PCy₃ (25.8 mg, 0.092 mmol), Pd₂(dba)₃ (42.1 mg, 0.046 mmol) and PdCl₂(dppf)-CH₂Cl₂ adduct (37.5 mg, 0.046 mmol) to afford after workup and purification using method-AE, ethyl 2-(2-((5-(1-aminoisoquinolin-5-yl)-2-cyclobutyl-2H-indazol-3-yl)methoxy)-4-(trifluoromethyl)phenyl)acetate (152a) (73 mg, 27.6% yield) as a yellow solid; MS (ES+): 575.2 (M+1).

### Step-2: Preparation of 2-(2-((5-(1-aminoisoquinolin-5-yl)˜2-cyclobutyl-2H-indazol-3-yl)methoxy)-4-(trifluoromethyl)phenyl)acetic acid (152b)

Compound 152b was prepared according to the procedure reported step-2 of scheme 1 from ethyl 2-(2-((5-(1-aminoisoquinolin-5-yl)-2-cyclobutyl-2H-indazol-3-yl)methoxy)-4-(trifluoromethyl)phenyl)acetate (152a) in THF (3 mL) using a solution of LiOH·H₂O (15.99 mg, 0.381 mmol) in water (1 mL) to afford after workup and purification using method-M, 2-(2-((5-(1-aminoisoquinolin-5-yl)-2-cyclobutyl-2H-indazol-3-yl)methoxy)-4-(trifluoromethyl)phenyl)acetic acid (152b) (13.2 mg, 19% yield) HCl salt as a white solid; ¹H NMR (300 MHz, DMSO-d₆) δ 13.05 (s, 1H, D₂O exchangeable), 12.33 (s, 1H, D₂O exchangeable), 9.09 (s, 2H, D₂O exchangeable), 8.60 (d, J=8.3 Hz, 1H), 8.01 (s, 1H), 7.95 (d, J=7.3 Hz, 1H), 7.92-7.80 (m, 2H), 7.66-7.54 (m, 2H), 7.45 (d, J=7.8 Hz, 1H), 7.37-7.28 (m, 2H), 6.97 (d, J=7.3 Hz, 1H), 5.74 (s, 2H), 5.42-5.28 (m, 1H), 3.58 (s, 2H), 2.82-2.68 (m, 4H), 1.98-1.84 (m, 2H); ¹⁹F NMR (282 MHz, DMSO-d₆) δ −60.71; MS (ES+): 547.2 (M+1).

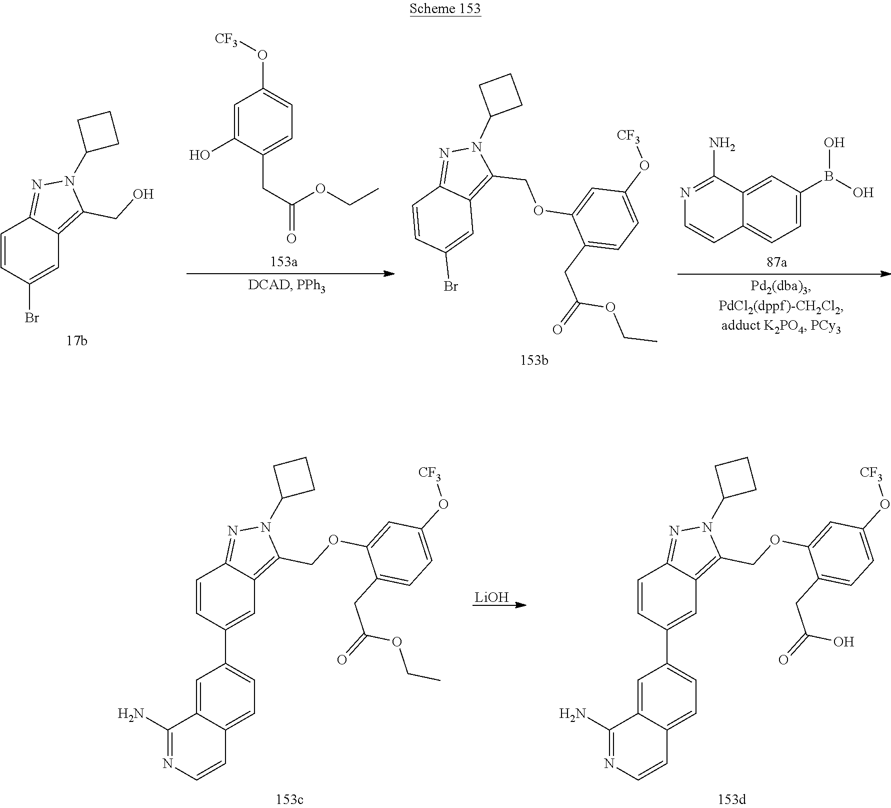

### Preparation of 2-(2-((5-(1-aminoisoquinolin-7-yl)-2-cyclobutyl-2H-indazol-3-yl)methoxy)-4-(trifluoromethoxy)phenyl)acetic acid (153d)

### Step-1: Preparation of ethyl 2-(2-((5-bromo-2-cyclobutyl-2H-indazol-3-yl)methoxy)-4-(trifluoromethoxy)phenyl)acetate (153b)

Compound 153b was prepared according to the procedure reported in step-2 of scheme 2, from (5-bromo-2-cyclobutyl-2H-indazol-3-yl)methanol (17b) (1 g, 3.56 mmol) in DCM (40 mL) using PPh₃ (1.026 g, 3.91 mmol), ethyl 2-(2-hydroxy-4-(trifluoromethoxy)phenyl)acetate (153a) (1.128 g, 4.27 mmol) and a solution of DCAD (1.437 g, 3.91 mmol) in DCM (20 mL) to afford after workup and purification using method-AV, ethyl 2-(2-((5-bromo-2-cyclobutyl-2H-indazol-3-yl)methoxy)-4-(trifluoromethoxy)phenyl)acetate (153b) (925 mg, 49.3% yield) as a white solid; MS (ES+): 527.1 (M+1).

### Step-2: Preparation of ethyl 2-(2-((5-(1-aminoisoquinolin-7-yl)-2-cyclobutyl-2H-indazol-3-yl)methoxy)-4-(trifluoromethoxy)phenyl)acetate (153c)

Compound 153c was prepared according to the procedure reported in step-5 of scheme 1 from ethyl 2-(2-((5-bromo-2-cyclobutyl-2H-indazol-3-yl)methoxy)-4-(trifluoromethoxy)phenyl)acetate (153b) (250 mg, 0.474 mmol) in dioxane/THF (12 mL, 1:1) using 1-aminoisoquinolin-7-ylboronic acid (87a) (116 mg, 0.616 mmol), K₃PO₄ (2M aqueous solution, 0.474 mL, 1.896 mmol), PCy₃ (26.6 mg, 0.095 mmol), Pd₂(dba)₃ (43.4 mg, 0.047 mmol) and PdCl₂(dppf)-CH₂Cl₂ adduct (38.7 mg, 0.047 mmol) to afford after workup and purification using method-AE, ethyl 2-(2-((5-(1-aminoisoquinolin-7-yl)-2-cyclobutyl-2H-indazol-3-yl)methoxy)-4-(trifluoromethoxy)phenyl)acetate (153c) (75 mg, 26.8% yield) as a yellow solid; MS (ES+): 591.2 (M+1).

### Step-3:Preparation of 2-(2-((5-(1-aminoisoquinolin-7-yl)-2-cyclobutyl-2H-indazol-3-yl)methoxy)-4-(trifluoromethoxy)phenyl)acetic acid (153d)

Compound 153d was prepared according to the procedure reported step-2 of scheme 1 from ethyl 2-(2-((5-(1-aminoisoquinolin-7-yl)-2-cyclobutyl-2H-indazol-3-yl)methoxy)-4-(trifluoromethoxy)phenyl)acetate (153c) in THF (3 mL) using a solution of LiOH·H₂O (15.99 mg, 0.381 mmol) in water (1 mL) to afford after workup and purification using method-M, 2-(2-((5-(1-aminoisoquinolin-7-yl)-2-cyclobutyl-2H-indazol-3-yl)methoxy)-4-(trifluoromethoxy)phenyl)acetic acid (153d) (6.4 mg, 9% yield) HCl salt as a white solid; ¹H NMR (300 MHz, DMSO-d₆) δ 13.06 (s, 1H), 12.26 (s, 1H), 9.08 (s, 1H), 8.95 (s, 1H), 8.50-8.40 (m, 2H), 8.06 (d, J=8.6 Hz, 1H), 7.90 (s, 2H), 7.68 (d, J=6.9 Hz, 1H), 7.41-7.33 (m, 2H), 7.29 (d, J=6.9 Hz, 1H), 6.99 (d, J=8.5 Hz, 1H), 5.71 (s, 2H), 5.39-5.26 (m, 1H), 3.53 (s, 2H), 2.86-2.60 (m, 4H), 1.99-1.86 (m, 2H); ¹⁹F NMR (282 MHZ, DMSO-d₆) δ −56.54; MS (ES+): 563.2 (M+1).

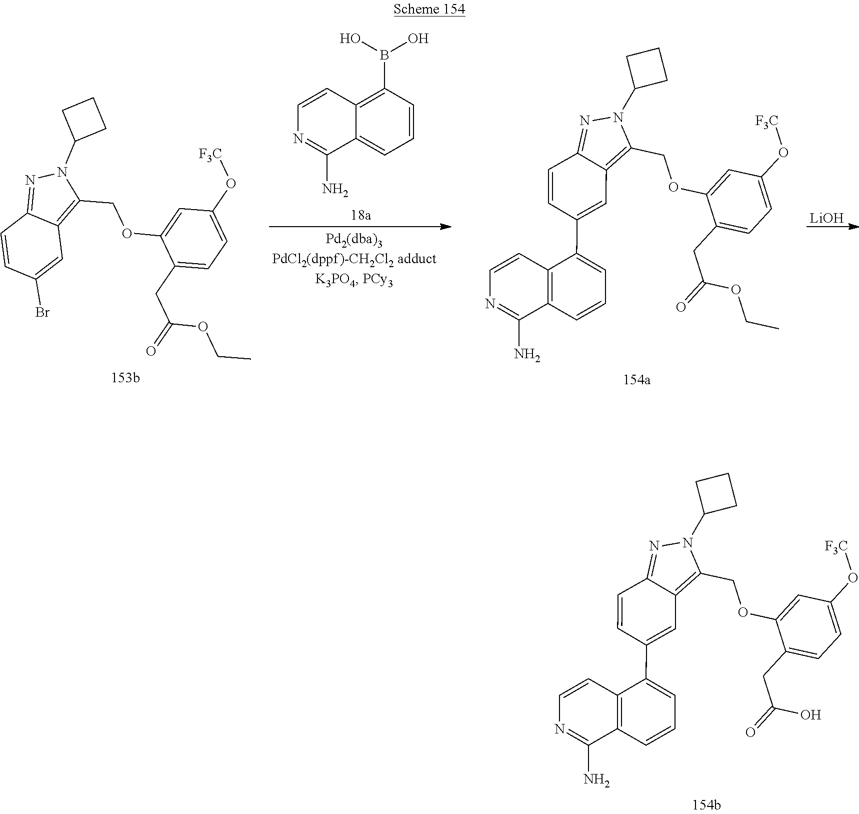

### Preparation of 2-(2-((5-(1-aminoisoquinolin-5-yl)-2-cyclobutyl-2H-indazol-3-yl)methoxy)-4-(trifluoromethoxy)phenyl)acetic acid (154b)

### Step-1: Preparation of ethyl 2-(2-((5-(1-aminoisoquinolin-5-yl)-2-cyclobutyl-2H-indazol-3-yl)methoxy)-4-(trifluoromethoxy)phenyl)acetate (154a)

Compound 154a was prepared according to the procedure reported in step-5 of scheme 1 from ethyl 2-(2-((5-bromo-2-cyclobutyl-2H-indazol-3-yl)methoxy)-4-(trifluoromethoxy)phenyl)acetate (153b) (250 mg, 0.474 mmol) in dioxane/THF (8 mL, 1:1) using (1-aminoisoquinolin-5-yl)boronic acid (18a) (89 mg, 0.474 mmol), K₃PO₄ (2M aqueous solution, 0.474 mL, 1.896 mmol), PCy₃ (26.6 mg, 0.095 mmol), Pd₂(dba)₃ (43.4 mg, 0.047 mmol) and PdCl₂(dppf)-CH₂Cl₂ adduct (38.7 mg, 0.047 mmol) to afford after workup and purification using method-AE, ethyl 2-(2-((5-(1-aminoisoquinolin-5-yl)-2-cyclobutyl-2H-indazol-3-yl)methoxy)-4-(trifluoromethoxy)phenyl)acetate (154a) (92 mg, 32.9% yield) as a yellow solid; MS (ES+): 591.2 (M+1).

### Step-2: Preparation of 2-(2-((5-(1-aminoisoquinolin-5-yl)-2-cyclobutyl-2H-indazol-3-yl)methoxy)-4-(trifluoromethoxy)phenyl)acetic acid (154b)

Compound 154b was prepared according to the procedure reported step-2 of scheme 1 from ethyl 2-(2-((5-(1-aminoisoquinolin-5-yl)-2-cyclobutyl-2H-indazol-3-yl)methoxy)-4-(trifluoromethoxy)phenyl)acetate (154a) in THF (3 mL) using a solution of LiOH·H₂O (19.61 mg, 0.467 mmol) in water (1 mL) to afford after workup and purification using method-M, 2-(2-((5-(1-aminoisoquinolin-5-yl)-2-cyclobutyl-2H-indazol-3-yl)methoxy)-4-(trifluoromethoxy)phenyl)acetic acid (154b) (13.7 mg, 16% yield) HCl salt as a white solid; JH NMR (300 MHz, DMSO-d₆) δ 13.04 (s, 1H, D₂O exchangeable), 12.26 (s, 1H, D₂O exchangeable), 9.04 (s, 2H, D₂O exchangeable), 8.59 (d, J=8.2 Hz, 1H), 7.99 (s, 1H), 7.97-7.91 (m, 1H), 7.91-7.80 (m, 2H), 7.62 (d, J=7.3 Hz, 1H), 7.40-7.25 (m, 3H), 7.02-6.89 (m, 2H), 5.66 (s, 2H), 5.41-5.26 (m, 1H), 3.51 (s, 2H), 2.85-2.67 (m, 4H), 2.01-1.80 (m, 2H); ¹⁹F NMR (282 MHz, DMSO-d₆) δ −56.64; MS (ES+): 563.2 (M+1).

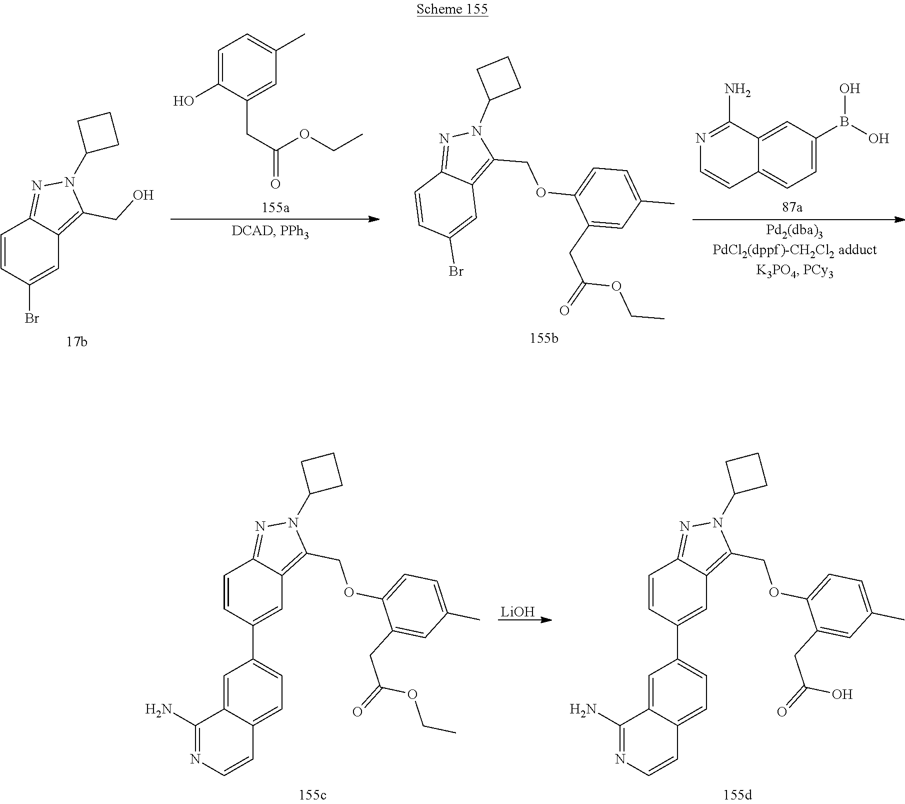

### Preparation of 2-(2-((5-(1-aminoisoquinolin-7-yl)-2-cyclobutyl-2H-indazol-3-yl)methoxy)-5-methylphenyl)acetic acid (155d)

### Step-1: Preparation of ethyl 2-(2-((5-bromo-2-cyclobutyl-2H-indazol-3-yl)methoxy)-5-methylphenyl)acetate (155b)

Compound 155b was prepared according to the procedure reported in step-2 of scheme 2, from (5-bromo-2-cyclobutyl-2H-indazol-3-yl)methanol (17b) (1 g, 3.56 mmol) in DCM (40 mL) using PPh₃ (1.026 g, 3.91 mmol), ethyl 2-(2-hydroxy-5-methylphenyl)acetate (155a) (0.829 g, 4.27 mmol; CAS #41873-67-0) and a solution of DCAD (1.437 g, 3.91 mmol) in DCM (20 mL) to afford after workup and purification using method-AV, ethyl 2-(2-((5-bromo-2-cyclobutyl-2H-indazol-3-yl)methoxy)-5-methylphenyl)acetate (155b) (725 mg, 44.6% yield) as a white solid; MS (ES+): 457.1 (M+1).

### Step-2: Preparation of ethyl 2-(2-((5-(1-aminoisoquinolin-7-yl)-2-cyclobutyl-2H-indazol-3-yl)methoxy)-5-methylphenyl)acetate (155c)

Compound 155c was prepared according to the procedure reported in step-5 of scheme 1 from ethyl 2-(2-((5-bromo-2-cyclobutyl-2H-indazol-3-yl)methoxy)-5-methylphenyl)acetate (155b) (250 mg, 0.547 mmol) in dioxane/THF (12 mL, 1:1) using 1-aminoisoquinolin-7-ylboronic acid (87a) (134 mg, 0.711 mmol), K₃PO₄ (2M aqueous solution, 0.547 mL, 2.186 mmol), PCy₃ (30.7 mg, 0.109 mmol), Pd₂(dba)₃ (50.1 mg, 0.055 mmol) and PdCl₂(dppf)-CH₂Cl₂ adduct (44.6 mg, 0.055 mmol) to afford after workup and purification using method-AE, ethyl 2-(2-((5-(1-aminoisoquinolin-7-yl)-2-cyclobutyl-2H-indazol-3-yl)methoxy)-5-methylphenyl)acetate (155c) (81 mg, 28.5% yield) as a yellow solid; MS (ES+): 521.2 (M+1).

### Step-3: Preparation of 2-(2-((5-(1-aminoisoquinolin-7-yl)-2-cyclobutyl-2H-indazol-3-yl)methoxy)-5-methylphenyl)acetic acid (155d)

Compound 155d was prepared according to the procedure reported step-2 of scheme 1 from ethyl 2-(2-((5-(1-aminoisoquinolin-7-yl)-2-cyclobutyl-2H-indazol-3-yl)methoxy)-5-methylphenyl)acetate (155c) in THF (3 mL) using a solution of LiOH·H₂O (19.59 mg, 0.467 mmol) in water (1 ml) to afford after workup and purification using method-M, 2-(2-(5-(1-aminoisoquinolin-7-yl)-2-cyclobutyl-2H-indazol-3-yl)methoxy)-5-methylphenyl)acetic acid (155d) (12.3 mg, 16% yield) HCl salt as a white solid; ¹H NMR (300 MHz, DMSO-d₆) δ 13.09 (s, 1H, D₂O exchangeable), 12.13 (s, 1H, D₂O exchangeable), 9.12 (s, 2H, D₂O exchangeable), 8.95 (s, 1H), 8.48-8.38 (m, 2H), 8.05 (d, J=8.4 Hz, 1H), 7.88 (s, 2H), 7.68 (d, J=6.9 Hz, 1H), 7.28 (d, J=7.0 Hz, 1H), 7.21 (d, J=8.4 Hz, 1H), 7.11 (d, 1H), 7.03 (s, 1H), 5.58 (s, 2H), 5.38-5.23 (m, 1H), 3.45 (s, 2H), 2.86-2.56 (m, 4H), 2.24 (s, 3H), 1.97-1.84 (m, 2H); MS (ES+): 493.2 (M+1).

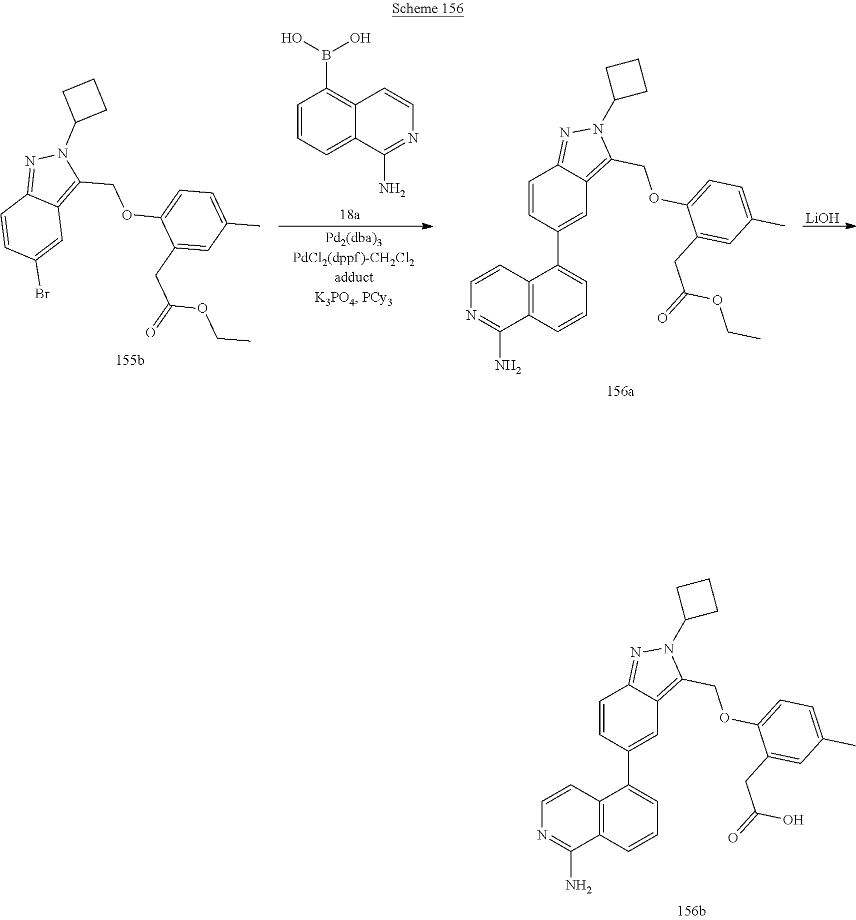

### Preparation of 2-(2-((5-(1-aminoisoquinolin-5-yl)-2-cyclobutyl-2H-indazol-3-yl)methoxy)-5-methylphenyl)acetic acid (156b)

### Step-1: Preparation of ethyl 2-(2-((5-(1-aminoisoquinolin-5-yl)-2-cyclobutyl-2H-indazol-3-yl)methoxy)-5-methylphenyl)acetate (156a)

Compound 156a was prepared according to the procedure reported in step-5 of scheme 1 from ethyl 2-(2-((5-bromo-2-cyclobutyl-2H-indazol-3-yl)methoxy)-5-methylphenyl)acetate (155b) (210 mg, 0.459 mmol) in dioxane/THF (8 mL, 1:1) using (I-aminoisoquinolin-5-yl)boronic acid (18a) (86 mg, 0.459 mmol), K₃PO₄ (2M aqueous solution, 0.459 mL, 1.837 mmol), PCy₃ (25.8 mg, 0.092 mmol), Pd₂(dba)₃ (42.0 mg, 0.046 mmol) and PdCl₂(dppf)-CH₂Cl₂ adduct (37.5 mg, 0.046 mmol) to afford after workup and purification using method-AE, ethyl 2-(2-((5-(1-aminoisoquinolin-5-yl)-2-cyclobutyl-2H-indazol-3-yl)methoxy)-5-methylphenyl)acetate (156a) (41 mg, 17.15% yield) as a yellow solid; MS (ES+): 521.2 (M+1).

### Step-2: Preparation of 2-(2-((5-(1-aminoisoquinolin-5-yl)-2-cyclobutyl-2H-indazol-3-yl)methoxy)-5-methylphenyl)acetic acid (156b)

Compound 156b was prepared according to the procedure reported step-2 of scheme 1 from ethyl 2-(2-(5-(1-aminoisoquinolin-5-yl)-2-cyclobutyl-2H-indazol-3-yl)methoxy)-5-methylphenyl)acetate (156a) (41 mg, 0.079 mmol) in THF (3 mL) using a solution of LiOH·H₂O (9.91 mg, 0.236 mmol) in water (I mL) to afford after workup and purification using method-M, 2-(2-((5-(1-aminoisoquinolin-5-yl)-2-cyclobutyl-2H-indazol-3-yl)methoxy)-5-methylphenyl)acetic acid (156b) (7.2 mg, 19% yield) HCl salt as a white solid; ¹H NMR (300 MHz, DMSO-d₆) (a mixture of rotamers) δ 13.07 (s, 1H, D₂O exchangeable), 12.12 (s, 1H, D₂O exchangeable), 9.09 (s, 2H, D₂O exchangeable), 8.60 (d, J=8.3 Hz, 1H), 7.99-7.92 (m, 2H), 7.92-7.80 (m, 2H), 7.63 (d, J=7.4 Hz, 1H), 7.32 (d, J=8.9 Hz, 1H), 7.19-7.11 (m, 1H), 7.11-7.04 (m, 1H), 7.04-6.95 (m, 2H), 5.58 and 5.54 (2s, 2H), 5.39-5.28 (m, 1H), 3.44 (s, 2H), 2.91-2.57 (m, 4H), 2.29 and 2.23 (2s, 3H), 1.99-1.86 (m, 2H); MS (ES+): 493.2 (M+1).

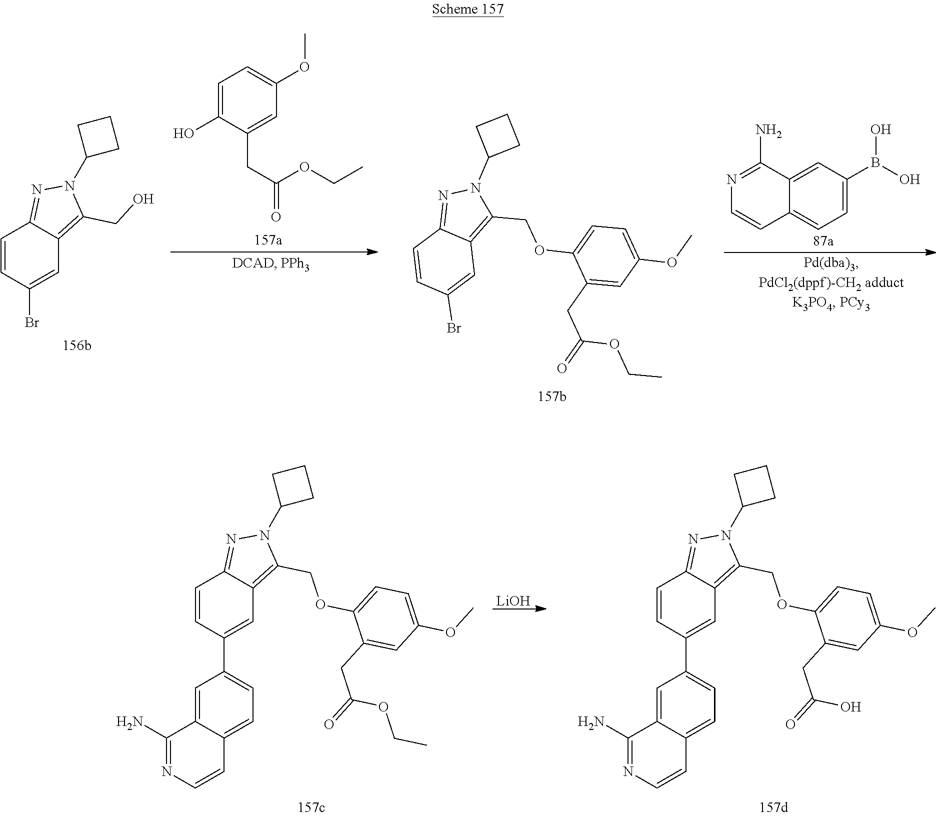

### Preparation of 2-(2-((5-(1-aminoisoquinolin-7-yl)-2-cyclobutyl-2H-indazol-3-yl)methoxy)-5-methoxyphenyl)acetic acid (157d)

### Step-1: Preparation of ethyl 2-(2-((5-bromo-2-cyclobutyl-2H-indazol-3-yl)methoxy)-5-methoxyphenyl)acetate (157b)

Compound 157b was prepared according to the procedure reported in step-2 of scheme 2, from (5-bromo-2-cyclobutyl-2H-indazol-3-yl)methanol (17b) (650 mg, 2.312 mmol) in DCM (15 mL) using PPh₃ (667 mg, 2.54 mmol), ethyl 2-(2-hydroxy-5-methoxyphenyl)acetate (157a) (583 mg, 2.77 mmol) and a solution of DCAD (934 mg, 2.54 mmol) in DCM to afford after workup and purification using method-AU, ethyl 2-(2-((5-bromo-2-cyclobutyl-2H-indazol-3-yl)methoxy)-5-methoxyphenyl)acetate (157b) (687 mg, 62.8% yield) as a white solid; MS (ES+): 473.1 (M+1).

### Step-2: Preparation of ethyl 2-(2-((5-(1-aminoisoquinolin-7-yl)-2-cyclobutyl-2H-indazol-3-yl)methoxy)-5-methoxyphenyl)acetate (157c)

Compound 157c was prepared according to the procedure reported in step-5 of scheme 1 from ethyl 2-(2-((5-bromo-2-cyclobutyl-2H-indazol-3-yl)methoxy)-5-methoxyphenyl)acetate (157b) (200 mg, 0.423 mmol) in dioxane/THF (12 mL, 1:1) using 1-aminoisoquinolin-7-ylboronic acid (87a) (103 mg, 0.549 mmol), K₃PO₄ (2M aqueous solution, 0.423 mL, 1.690 mmol), PCy₃ (23.70 mg, 0.085 mmol), Pd₂(dba)₃ (38.7 mg, 0.042 mmol) and PdCl₂(dppf)-CH₂Cl₂ adduct (34.5 mg, 0.042 mmol) to afford after workup and purification using method-AE, ethyl 2-(2-((5-(1-aminoisoquinolin-7-yl)-2-cyclobutyl-2H-indazol-3-yl)methoxy)-5-methoxyphenyl)acetate (157c) (43 mg, 18.97% yield) as a yellow solid; MS (ES+): 537.3 (M+1).

### Step-3: Preparation of 2-(2-((5-(1-aminoisoquinolin-7-yl)-2-cyclobutyl-2H-indazol-3-yl)methoxy)-5-methoxyphenyl)acetic acid (157d)

Compound 157d was prepared according to the procedure reported step-2 of scheme 1 from ethyl 2-(2-((5-(1-aminoisoquinolin-7-yl)-2-cyclobutyl-2H-indazol-3-yl)methoxy)-5-methoxyphenyl)acetate (157c) in THF (3 mL) using a solution of LiOH·H₂O (10.09 mg, 0.24 mmol) in water (1 mL) to afford after workup and purification using method-M, 2-(2-((5-(1-aminoisoquinolin-7-yl)-2-cyclobutyl-2H-indazol-3-yl)methoxy)-5-methoxyphenyl)acetic acid (157d) (3.2 mg, 8% yield) HCl salt as a white solid; ¹H NMR (300 MHz, DMSO-d₆) δ 13.07 (s, 1H, D₂O exchangeable), 12.20 (s, 1H, D₂O exchangeable), 9.10 (s, 1H, D₂O exchangeable), 8.96 (s, 1H), 8.45 (d, J=8.8 Hz, 1H), 8.40 (s, 1H), 8.06 (d, J=8.6 Hz, 1H), 7.89 (s, 2H), 7.68 (d, J=7.0 Hz, 1H), 7.63-7.50 (m, 1H), 7.27 (dd, J=13.2, 7.7 Hz, 2H), 6.92-6.82 (m, 2H), 5.56 (s, 2H), 5.37-5.27 (m, 1H), 3.71 (s, 3H), 3.49 (s, 2H), 2.86-2.58 (m, 4H), 1.97-1.86 (m, 2H); MS (ES+): 509.2 (M+1).

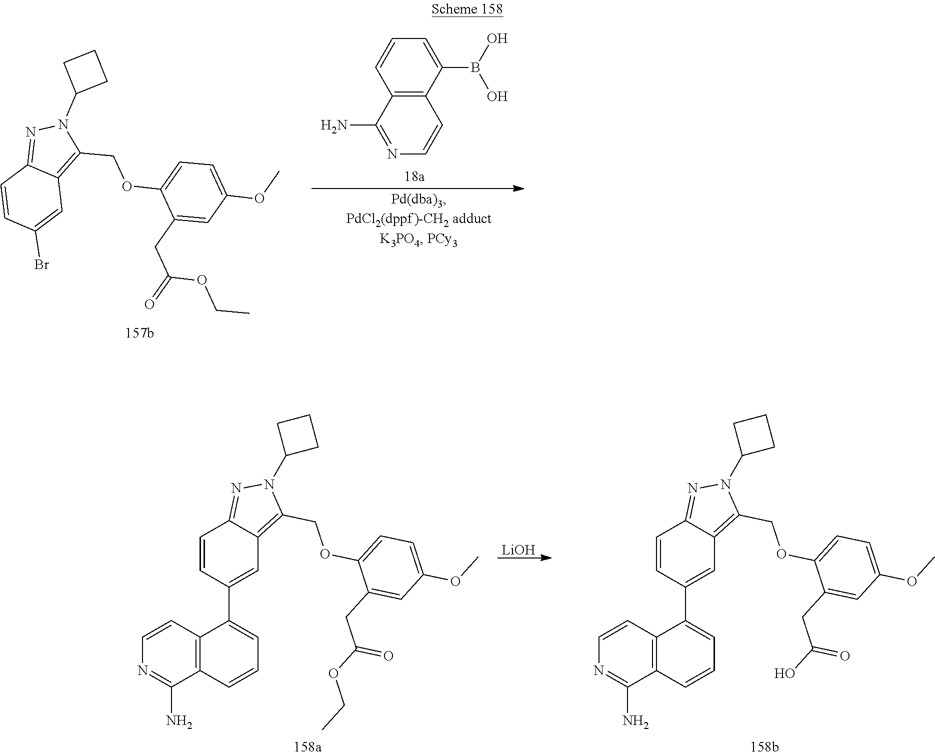

### Preparation of 2-(2-((5-(1-aminoisoquinolin-5-yl)-2-cyclobutyl-2H-indazol-3-yl)methoxy)-5-methoxyphenyl)acetic acid (158b)

### Step-1: Preparation of ethyl 2-(2-((5-(1-aminoisoquinolin-5-yl)-2-cyclobutyl-2H-indazol-3-yl)methoxy)-5-methoxyphenyl)acetate (158a)

Compound 158a was prepared according to the procedure reported in step-5 of scheme 1 from ethyl 2-(2-((5-bromo-2-cyclobutyl-2H-indazol-3-yl)methoxy)-5-methoxyphenyl)acetate (157b) (200 mg, 0.423 mmol) in dioxane/THF (8 mL, 1:1) using (1-aminoisoquinolin-5-yl)boronic acid (18a) (79 mg, 0.423 mmol). K₃PO₄ (2M aqueous solution, 0.423 mL, 1.690 mmol), PCy₃ (23.70 mg, 0.085 mmol), Pd₂(dba)₃ (38.7 mg, 0.042 mmol) and PdCl₂(dppf)-CH₂Cl₂ adduct (34.5 mg, 0.042 mmol) to afford after workup and purification using method-AE, ethyl 2-(2-((5-(1-aminoisoquinolin-5-yl)-2-cyclobutyl-2H-indazol-3-yl)methoxy)-5-methoxyphenyl)acetate (158a) (47 mg, 20.73% yield) as a yellow solid; MS (ES+): 537.3 (M+1).

### Step-2: Preparation of 2-(2-((5-(1-aminoisoquinolin-5-yl)-2-cyclobutyl-2H-indazol-3-yl)methoxy)-5-methoxyphenyl)acetic acid (158b)

Compound 158b was prepared according to the procedure reported step-2 of scheme 1 from ethyl 2-(2-((5-(1-aminoisoquinolin-5-yl)-2-cyclobutyl-2H-indazol-3-yl)methoxy)-5-methoxyphenyl)acetate (158a) (47 mg, 0.088 mmol) in THF (3 mL) using a solution of LiOH·H₂O (11.03 mg, 0.263 mmol) in water (1 mL) to afford after workup and purification using method-M, 2-(2-((5-(1-aminoisoquinolin-5-yl)-2-cyclobutyl-2H-indazol-3-yl)methoxy)-5-methoxyphenyl)acetic acid (158b) (5.2 mg, 11.67% yield) HCl salt as a light tan solid; ¹H NMR (300 MHz, DMSO-d₆) δ 13.15 (s, 1H, DO exchangeable), 12.21 (s, 1H, D₂O exchangeable), 9.15 (s, 2H, D₂O exchangeable), 8.62 (d, J=8.4 Hz, 1H), 8.00-7.79 (m, 4H), 7.63 (d, J=7.3 Hz, 1H), 7.32 (d, J=8.8 Hz, 1H), 7.19 (d, J=9.4 Hz, 1H), 6.98 (d, J=7.2 Hz, 1H), 6.87-6.78 (m, 2H), 5.51 (s, 2H), 5.44-5.30 (m, 1H), 3.70 (s, 3H), 3.45 (s, 2H), 2.91-2.56 (m, 4H), 2.00-1.87 (m, 2H); MS (ES+): 509.2 (M+1); MS (ES−): 507.1 (M−1).

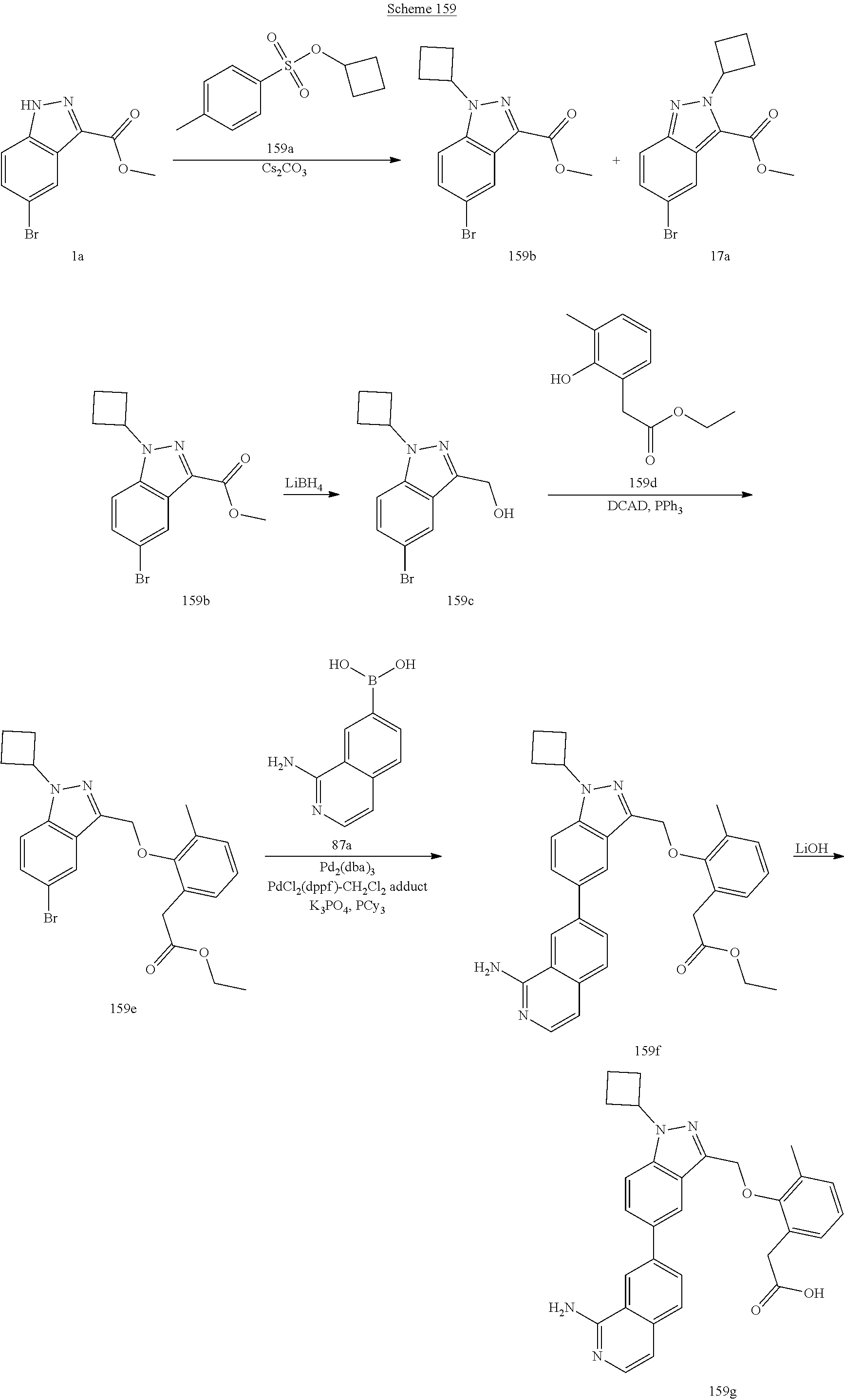

### Preparation of 2-(2-((5-(1-aminoisoquinolin-7-yl)-1-cyclobutyl-1H-indazol-3-yl)methoxy)-3-methylphenyl)acetic acid (159g)

### Step-1: Preparation methyl 5-bromo-1-cyclobutyl-1H-indazole-3-carboxylate (159b)

Compound 159b was prepared according to the procedure reported in step-2 of scheme 86 from methyl 5-bromo-1H-indazole-3-carboxylate (1a) (12.08 g, 47.3 mmol) in DMF (240 mL) using Cs₂CO₃ (30.9 g, 95 mmol) and cyclobutyl 4-methylbenzenesulfonate (159a) (15 g, 66.3 mmol, prepared from cyclobutanol using DMAP, TEA and p-toluenesulfonylchloride as reported in step-1 of scheme 86) to afford after workup and purification using method-A, methyl 5-bromo-1-cyclobutyl-1H-indazole-3-carboxylate (159b) (2.0 g, 13.66% yield) as a white solid and methyl 5-bromo-2-cyclobutyl-2H-indazole-3-carboxylate (17a) as a white solid; MS (ES+): 331.0 (M+Na).

### Step-2: Preparation (5-bromo-1-cyclobutyl-1H-indazol-3-yl)methanol (159c)

Compound 159c was prepared according to the procedure reported in step-2 of scheme 119 from methyl 5-bromo-1-cyclobutyl-1H-indazole-3-carboxylate (159b) (10 g, 32.3 mmol) in THF (25 mL) using LiBH₄ (40.4 mL, 81 mmol) and MeOH (3.27 mL, 81 mmol) to afford after workup and purification using method-AW. (5-bromo-1-cyclobutyl-1H-indazol-3-yl)methanol (159c) (7.7 g, 85% yield) as a clear oil; MS (ES+): 281.0, 283.0 (M+1).

### Step-3: Preparation ethyl 2-(2-((5-bromo-1-cyclobutyl-1H-indazol-3-yl)methoxy)-3-methylphenyl)acetate (159e)

Compound 159e was prepared according to the procedure reported in step-2 of scheme 2, from (5-bromo-1-cyclobutyl-1H-indazol-3-yl)methanol (159c) (500 mg, 1.778 mmol) in DCM (10 mL) using PPh₃ (513 mg, 1.956 mmol), ethyl 2-(2-hydroxy-3-methylphenyl)acetate (159d) (415 mg, 2.134 mmol; CAS #1261620-23-8)) and a solution of DCAD (718 mg, 1.956 mmol) in DCM to afford after workup and purification using method-AX, ethyl 2-(2-((5-bromo-1-cyclobutyl-1H-indazol-3-yl)methoxy)-3-methylphenyl)acetate (159e) (520 mg, 63.9% yield) as a white solid; MS (ES+): 557.1, 459.1 (M+1).

### Step-4: Preparation ethyl 2-(2-((5-(1-aminoisoquinolin-7-yl)-1-cyclobutyl-1H-indazol-3-yl)methoxy)-3-methylphenyl)acetate (159f)

Compound 159f was prepared according to the procedure reported in step-5 of scheme 1 from ethyl 2-(2-((5-bromo-1-cyclobutyl-1H-indazol-3-yl)methoxy)-3-methylphenyl)acetate (159e) (255 mg, 0.558 mmol) in dioxane/MeTHF (10 mL, 1:1) using 1-aminoisoquinolin-7-ylboronic acid (87a) (136 mg, 0.725 mmol), K₃PO₄ (2M aqueous solution, 0.558 mL, 2.230 mmol). PCy₃ (31.3 mg, 0.112 mmol). Pd₂(dba)₃ (51.1 mg, 0.056 mmol) and PdCl₂(dppf)-CH₂Cl₂ adduct (45.5 mg, 0.056 mmol) to afford after workup and purification using method-AE, ethyl 2-(2-((5-(1-aminoisoquinolin-7-yl)-1-cyclobutyl-1H-indazol-3-yl)methoxy)-3-methylphenyl)acetate (159f) (143 mg, 49.3% yield) as a yellow solid; MS (ES+): 521.20 MS (M+1).

### Step-5: Preparation of 2-(2-((5-(1-aminoisoquinolin-7-yl)-1-cyclobutyl-1H-indazol-3-yl)methoxy)-3-methylphenyl)acetic acid (159g)

Compound 159g was prepared according to the procedure reported step-2 of scheme 1 from ethyl 2-(2-((5-(1-aminoisoquinolin-7-yl)-1-cyclobutyl-1H-indazol-3-yl)methoxy)-3-methylphenyl)acetate (159f) (143 mg, 0.275 mmol) in THF (3 mL), using a solution of LiOH·H₂O (34.6 mg, 0.824 mmol) in water (1 mL) to afford after workup and purification using method-M, 2-(2-((5-(1-aminoisoquinolin-7-yl)-1-cyclobutyl-1H-indazol-3-yl)methoxy)-3-methylphenyl)acetic acid (159g) (43.3 mg, 32.0% yield) HCl salt as a white solid; 1H NMR (300 MHz, DMSO-d₆) δ 13.14 (s, 1H, D₂O exchangeable), 12.45 (s, 1H, D₂O exchangeable), 9.08 (s, 2H, D₂O exchangeable), 8.94 (s, 1H), 8.36 (d, J=8.9 Hz, 1H), 8.28 (s, 1H), 8.12-7.96 (m, 2H), 7.96-7.88 (m, 1H), 7.76-7.65 (m, 1H), 7.35-7.22 (m, 1H), 7.18-7.08 (m, 2H), 7.08-6.92 (m, 1H), 5.50-5.32 (m, 1H), 5.26 (s, 2H), 3.67 (s, 2H), 2.79-2.60 (m, 2H), 2.59-2.53 (m, 2H), 2.36-2.21 (m, 3H), 2.02-1.81 (m, 2H); MS (ES+): 493.2 (M+1).

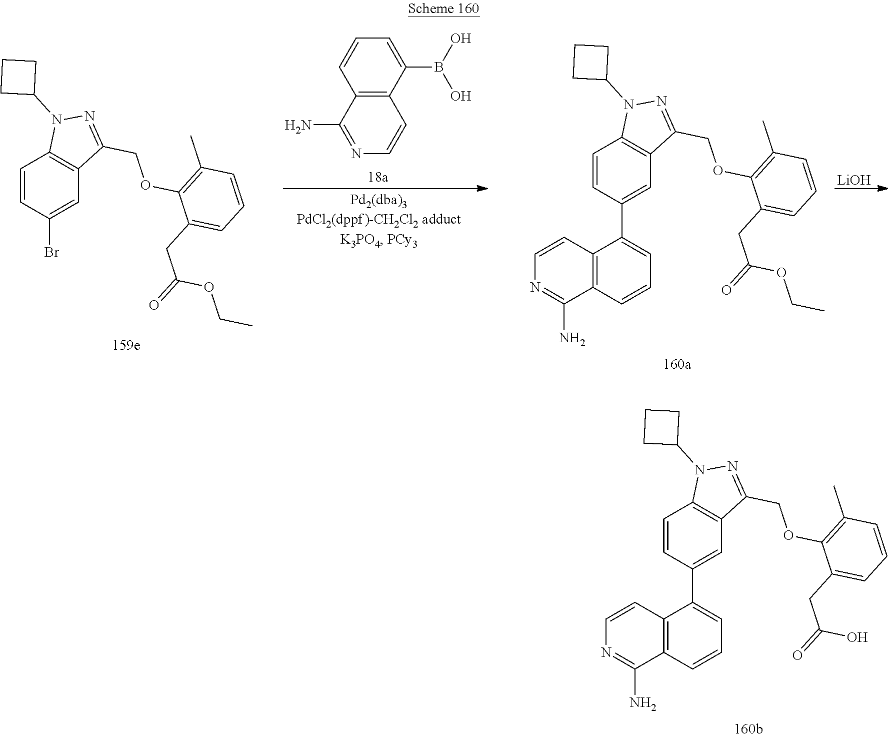

### Preparation of 2-(2-((5-(1-aminoisoquinolin-5-yl)-1-cyclobutyl-1H-indazol-3-yl)methoxy)-3-methylphenyl)acetic acid (160b)

### Step-1: Preparation of ethyl 2-(2-((5-(1-aminoisoquinolin-5-yl)-1-cyclobutyl-1H-indazol-3-yl)methoxy)-3-methylphenyl)acetate (160a)

Compound 160a was prepared according to the procedure reported in step-5 of scheme 1 from ethyl 2-(2-((5-bromo-1-cyclobutyl-1H-indazol-3-yl)methoxy)-3-methylphenyl)acetate (159e) (255 mg, 0.558 mmol) in dioxane/Me THF (25 mL, 1:1) using (1-aminoisoquinolin-5-yl)boronic acid (18a) (105 mg, 0.558 mmol), K₃PO₄ (2M aqueous solution, 0.558 mL, 2.230 mmol), PCy₃ (31.3 mg, 0.112 mmol), Pd₂(dba)₃ (51.1 mg, 0.056 mmol) and PdCl₂(dppf)-CH₂Cl₂ adduct (45.5 mg, 0.056 mmol) to afford after workup and purification using method-AE, ethyl 2-(2-((5-(1-aminoisoquinolin-5-yl)-1-cyclobutyl-1H-indazol-3-yl)methoxy)-3-methylphenyl)acetate (160a) (140 mg, 48.2% yield) as a yellow solid; MS (ES+): 521.20 (M+1).

### Step-2: Preparation of 2-(2-((5-(1-aminoisoquinolin-5-yl)-1-cyclobutyl-1H-indazol-3-yl)methoxy)-3-methylphenyl)acetic acid (160b)

Compound 160b was prepared according to the procedure reported step-2 of scheme 1 from ethyl 2-(2-((5-(1-aminoisoquinolin-5-yl)-1-cyclobutyl-1H-indazol-3-yl)methoxy)-3-methylphenyl)acetate (160a) (140 mg, 0.269 mmol) in THF (3 mL) using a solution of LiOH·H₂O (33.9 mg, 0.807 mmol) in water (1 mL) to afford after workup and purification using method-M, 2-(2-((5-(1-aminoisoquinolin-5-yl)-1-cyclobutyl-1H-indazol-3-yl)methoxy)-3-methylphenyl)acetic acid (160b) (43.3 mg, 0.088 mmol, 32.7% yield) HCl salt as a white solid; ¹H NMR (300 MHz, DMSO-d₆) δ 13.26 (s, 1H, D₂O exchangeable), 12.24 (s, 1H, D₂O exchangeable), 9.04 (s, 2H, D₂O exchangeable), 8.61 (d, J=8.0 Hz, 1H), 8.01-7.77 (m, 4H), 7.74-7.58 (m, 1H), 7.54-7.40 (m, 1H), 7.18-7.04 (m, 2H), 7.04-6.86 (m, 2H), 5.51-5.29 (m, 1H), 5.21 (s, 2H), 3.58 (d, J=3.5 Hz, 2H), 2.82-2.53 (m, 4H), 2.35-2.19 (m, 3H), 2.03-1.80 (m, 2H); MS (ES+): 493.2 (M+1); MS (ES−): 491.1 (M−1).

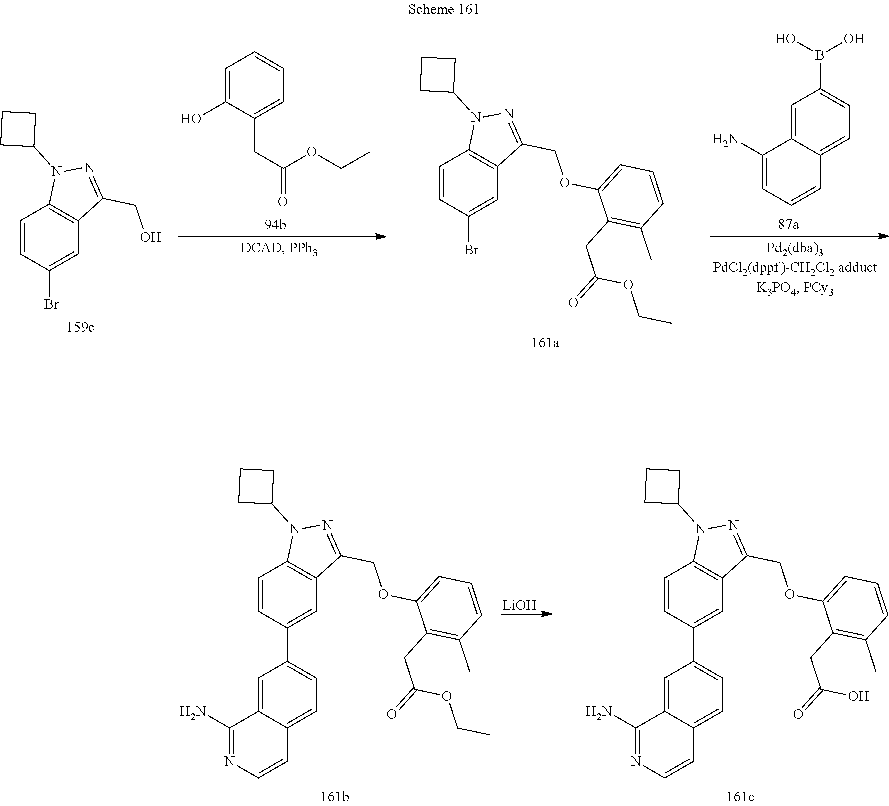

### Preparation of 2-(2-((5-(1-aminoisoquinolin-7-yl)-1-cyclobutyl-1H-indazol-3-yl)methoxy)-6-methylphenyl)acetic acid (161c)

### Step-1: Preparation ethyl 2-(2-((5-bromo-1-cyclobutyl-1H-indazol-3-yl)methoxy)-6-methylphenyl)acetate (161a)

Compound 161a was prepared according to the procedure reported in step-2 of scheme 2, from (5-bromo-1-cyclobutyl-1H-indazol-3-yl)methanol (159c) (700 mg, 2.49 mmol) in DCM (10 mL) using PPh₃ (718 mg, 2.74 mmol), ethyl 2-(2-hydroxy-6-methylphenyl)acetate (94b) (580 mg, 2.99 mmol) and a solution of DCAD (1006 mg, 2.74 mmol) in DCM (10 mL) to afford after workup and purification using method-AX, ethyl 2-(2-((5-bromo-1-cyclobutyl-1H-indazol-3-yl)methoxy)-6-methylphenyl)acetate (161a) (420 mg, 0.918 mmol, 36.9% yield) as a white solid; MS (ES+): 457.1, 459.1 (M+1).

### Step-2: Preparation ethyl 2-(2-((5-(1-aminoisoquinolin-7-yl)-1-cyclobutyl-1H-indazol-3-yl)methoxy)-6-methylphenyl)acetate (161b)

Compound 161b was prepared according to the procedure reported in step-5 of scheme 1 from ethyl 2-(2-((5-bromo-1-cyclobutyl-1H-indazol-3-yl)methoxy)-6-methylphenyl)acetate (161a) (210 mg, 0.459 mmol) in dioxane/MeTHF (10 mL, 1:1) using 1-aminoisoquinolin-7-ylboronic acid (87a) (112 mg, 0.597 mmol), K₃PO₄ (2M aqueous solution, 0.459 mL, 1.837 mmol), PCy₃ (25.8 mg, 0.092 mmol), Pd₂(dba)₃ (42.0 mg, 0.046 mmol) and PdCl₂(dppf)-CH₂Cl₂ adduct (37.5 mg, 0.046 mmol) to afford after workup and purification using method-AE, ethyl 2-(2-((5-(1-aminoisoquinolin-7-yl)-1-cyclobutyl-1H-indazol-3-yl)methoxy)-6-methylphenyl)acetate (161b) (108 mg, 45.2% yield) as a yellow solid; MS (ES+): 521.2 (M+1).

### Step-3: Preparation of 2-(2-((5-(1-aminoisoquinolin-7-yl)-1-cyclobutyl-1H-indazol-3-yl)methoxy)-6-methylphenyl)acetic acid (161c)

Compound 161c was prepared according to the procedure reported step-2 of scheme 1 from ethyl 2-(2-((5-(1-aminoisoquinolin-7-yl)-1-cyclobutyl-1H-indazol-3-yl)methoxy)-6-methylphenyl)acetate (161b) (108 mg, 0.207 mmol) in THF (3 mL) using a solution of LiOH·H₂O (26.1 mg, 0.622 mmol) in water (1 mL) to afford after workup and purification using method-M, 2-(2-((5-(1-aminoisoquinolin-7-yl)-1-cyclobutyl-1H-indazol-3-yl)methoxy)-6-methylphenyl)acetic acid (161c) (32.8 mg, 32.1% yield) HCl salt as a white solid; 1H NMR (300 MHz, DMSO-d₆) δ 12.84 (s, 1H, D₂O exchangeable), 12.19 (s, 1H, D₂O exchangeable), 9.03 (s, 2H, D₂O exchangeable), 8.93 (d, J=3.8 Hz, 1H), 8.44-8.32 (m, 1H), 8.29 (s, 1H), 8.10-7.95 (m, 2H), 7.95-7.86 (m, 1H), 7.76-7.65 (m, 1H), 7.32-7.23 (m, 1H), 7.16 (s, 2H), 6.84 (d, J=5.5 Hz, 1H), 5.49 (d, J=3.4 Hz, 2H), 5.44-5.31 (m, 1H), 3.59 (s, 2H), 2.81-2.54 (m, 4H), 2.27-2.16 (m, 3H), 2.01-1.81 (m, 2H); MS (ES+): 493.2 (M+1); (ES−): 491.2 (M−1).

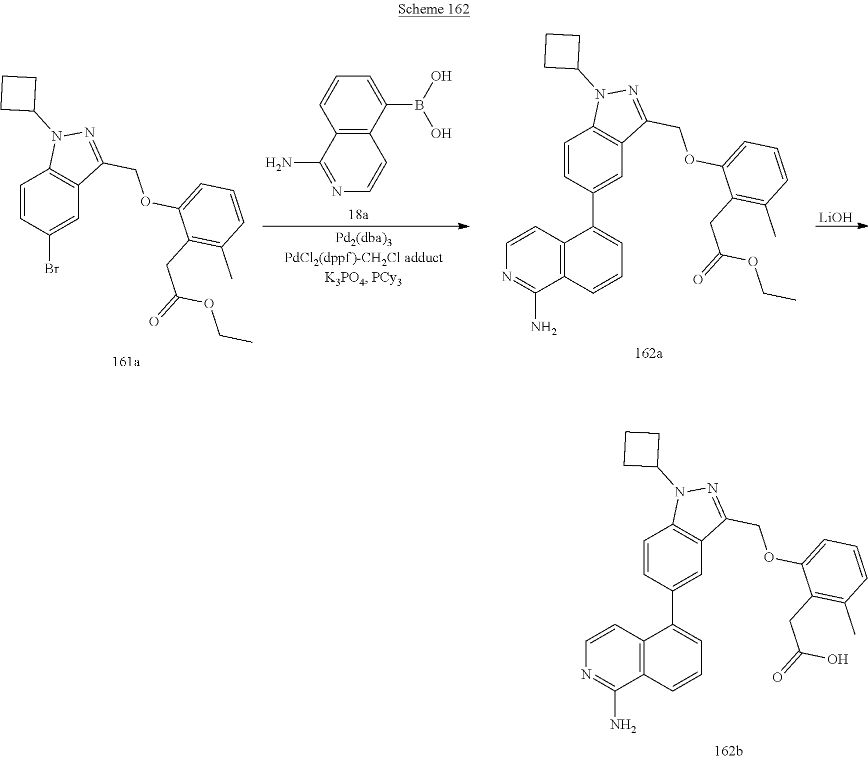

### Preparation of 2-(2-((5-(1-aminoisoquinolin-5-yl)-1-cyclobutyl-1H-indazol-3-yl)methoxy)-6-methylphenyl)acetic acid (162b)

### Step-1: Preparation ethyl 2-(2-((5-(1-aminoisoquinolin-5-yl)-1-cyclobutyl-1H-indazol-3-yl)methoxy)-6-methylphenyl)acetate (162a)

Compound 162a was prepared according to the procedure reported in step-5 of scheme 1 from ethyl 2-(2-((S-bromo-1-cyclobutyl-1H-indazol-3-yl)methoxy)-6-methylphenyl)acetate (161a) (0.21 g, 0.459 mmol) in dioxane/MeTHF (25 mL, 9:1) using (1-aminoisoquinolin-5-yl)boronic acid (18a) (0.086 g, 0.459 mmol), K₃PO₄ (2M aqueous solution, 0.459 mL, 1.837 mmol), PCy₃ (25.8 mg, 0.092 mmol), Pd₂(dba)₃ (42.0 mg, 0.046 mmol) and PdCl₂(dppf)-CH₂Cl₂ adduct (37.5 mg, 0.046 mmol) to afford after workup and purification using method-AE, ethyl 2-(2-((5-(1-aminoisoquinolin-5-yl)-1-cyclobutyl-1H-indazol-3-yl)methoxy)-6-methylphenyl)acetate (162a) (133 mg, 55.6% yield) as a yellow solid; MS (ES+): 521.2 (M+1).

### Step-2: Preparation 2-(2-((5-(1-aminoisoquinolin-5-yl)-1-cyclobutyl-1H-indazol-3-yl)methoxy)-6-methylphenyl)acetic acid (162b)

Compound 162b was prepared according to the procedure reported step-2 of scheme 1 from ethyl 2-(2-((5-(1-aminoisoquinolin-5-yl)-1-cyclobutyl-1H-indazol-3-yl)methoxy)-6-methylphenyl)acetate (162a) (133 mg, 0.255 mmol) in THF (3 mL) using a solution of LiOH·H₂O (32.2 mg, 0.766 mmol) in water (I mL) to afford after workup and purification using method-M, 2-(2-((5-(1-aminoisoquinolin-S-yl)-1-cyclobutyl-1H-indazol-3-yl)methoxy)-6-methylphenyl)acetic acid (162b) (33.6 mg, 26.7% yield) HCl salt as a white solid; ¹H NMR (300 MHz, DMSO-d₆) δ 13.22 (s, 1H, D₂O exchangeable), 12.05 (s, 1H, D₂O exchangeable), 9.06 (s, 2H, D₂O exchangeable), 8.69-8.48 (m, 1H), 8.00-7.76 (m, 4H), 7.70-7.57 (m, 1H), 7.55-7.39 (m, 1H), 7.13 (d, J=4.2 Hz, 2H), 7.02-6.91 (m, 1H), 6.81 (s, 1H), 5.45 (d, J=3.4 Hz, 2H), 5.42-5.29 (m, 1H), 3.55 (s, 2H), 2.82-2.54 (m, 4H), 2.26-2.11 (m, 3H), 2.02-1.82 (m, 2H); MS (ES+): 493.2 (M+1); MS (ES−): 491.1 (M−1)

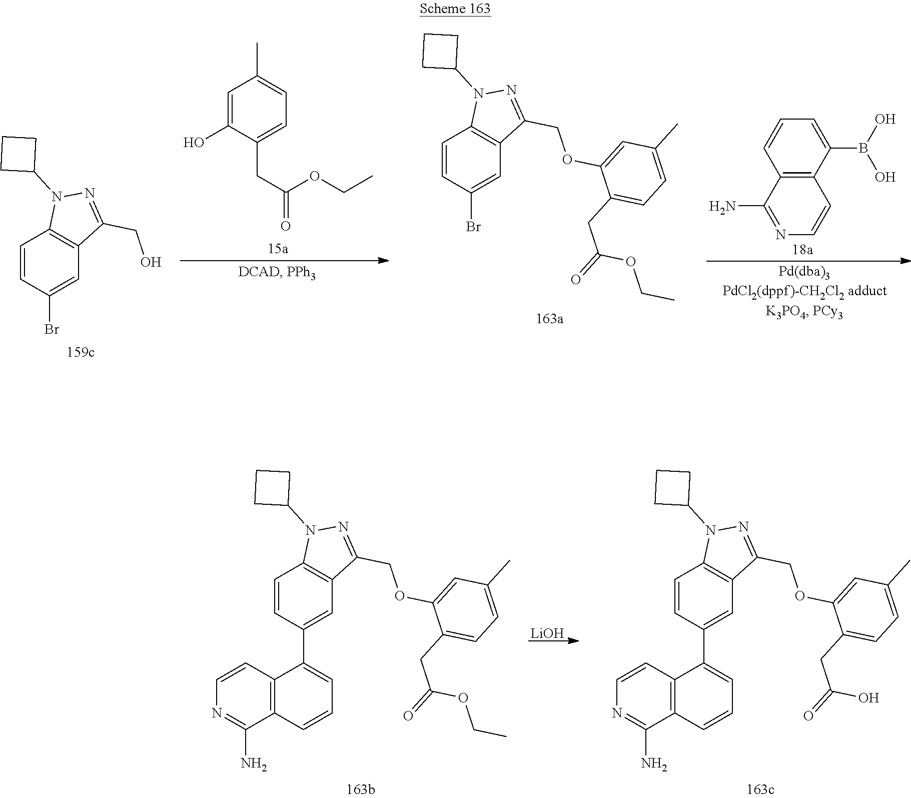

### Preparation of 2-(2-((5-(1-aminoisoquinolin-5-yl)-1-cyclobutyl-1H-indazol-3-yl)methoxy)-4-methylphenyl)acetic acid (163c)

### Step-1: Preparation ethyl 2-(2-((5-bromo-1-cyclobutyl-1H-indazol-3-yl)methoxy)-4-methylphenyl)acetate (163a)

Compound 163a was prepared according to the procedure reported in step-2 of scheme 2, from (5-bromo-1-cyclobutyl-1H-indazol-3-yl)methanol (159c) (500 mg, 1.778 mmol) in DCM (10 mL) using PPh₃ (513 mg, 1.956 mmol), ethyl 2-(2-hydroxy-4-methylphenyl)acetate (15a) (415 mg, 2.134 mmol) and a solution of DCAD (718 mg, 1.956 mmol) in DCM (10 mL) to afford after workup and purification using method-AU, ethyl 2-(2-((5-bromo-1-cyclobutyl-1H-indazol-3-yl)methoxy)-4-methylphenyl)acetate (163a) (525 mg, 64.5% yield) as a white solid; MS (ES+): 457.1, 459.1 (M+1).

### Step-2: Preparation ethyl 2-(2-((5-(1-aminoisoquinolin-5-yl)-1-cyclobutyl-1H-indazol-3-yl)methoxy)-4-methylphenyl)acetate (163b)

Compound 163b was prepared according to the procedure reported in step-5 of scheme 1 from ethyl 2-(2-((5-bromo-1-cyclobutyl-1H-indazol-3-yl)methoxy)-4-methylphenyl)acetate (163a) (255 mg, 0.558 mmol) in dioxane/MeTHF (10 mL, 1:1) using (1-aminoisoquinolin-5-yl)boronic acid (18a) (105 mg, 0.558 mmol), K₃PO₄ (2M aqueous solution, 0.558 mL, 2.230 mmol), PCy₃ (31.3 mg, 0.112 mmol), Pd₂(dba)₃ (51.1 mg, 0.056 mmol) and PdCl₂(dppf)-CH₂Cl₂ adduct (45.5 mg, 0.056 mmol) to afford after workup and purification using method-AE, ethyl 2-(2-((5-(1-aminoisoquinolin-5-yl)-1-cyclobutyl-1H-indazol-3-yl)methoxy)-4-methylphenyl)acetate (163b) (137 mg, 47.2% yield) as a yellow solid; (ES+): 521.2 (M+1).

### Step-3: Preparation of 2-(2-((5-(1-aminoisoquinolin-5-yl)-1-cyclobutyl-1H-indazol-3-yl)methoxy)-4-methylphenyl)acetic acid (163c)

Compound 163c was prepared according to the procedure reported step-2 of scheme 1 from ethyl 2-(2-((5-(1-aminoisoquinolin-5-yl)-1-cyclobutyl-1H-indazol-3-yl)methoxy)-4-methylphenyl)acetate (163b) (137 mg, 0.263 mmol) in THF (3 mL) using a solution of LiOH·H₂O (33.1 mg, 0.789 mmol) in water (1 mL) to afford after workup and purification using method-M, 2-(2-((5-(1-aminoisoquinolin-5-yl)-1-cyclobutyl-1H-indazol-3-yl)methoxy)-4-methylphenyl)acetic acid (163c) (32.1 mg, 24.77% yield) HCl salt as a white solid; ¹H NMR (300 MHz, DMSO-d₆) δ 13.17 (s, 1H, D₂O exchangeable), 11.97 (s, 1H, D₂O exchangeable), 9.08 (s, 2H, D₂O exchangeable), 8.60 (d, J=8.4 Hz, 1H), 7.95 (d, J=7.3 Hz, 1H), 7.92-7.80 (m, 3H), 7.62 (d, J=7.3 Hz, 1H), 7.47 (dd, J=8.8, 1.5 Hz, 1H), 7.13 (d, J=1.5 Hz, 1H), 7.01 (dd, J=16.2, 7.4 Hz, 2H), 6.72 (d, J=7.3 Hz, 1H), 5.46 (s, 2H), 5.43-5.27 (m, 1H), 3.42 (s, 2H), 2.80-2.61 (m, 2H), 2.59-2.53 (m, 2H), 2.29 (s, 3H), 2.03-1.83 (m, 2H); MS (ES+): 493.2 (M+1); MS (ES−): 491.1 (M−1).

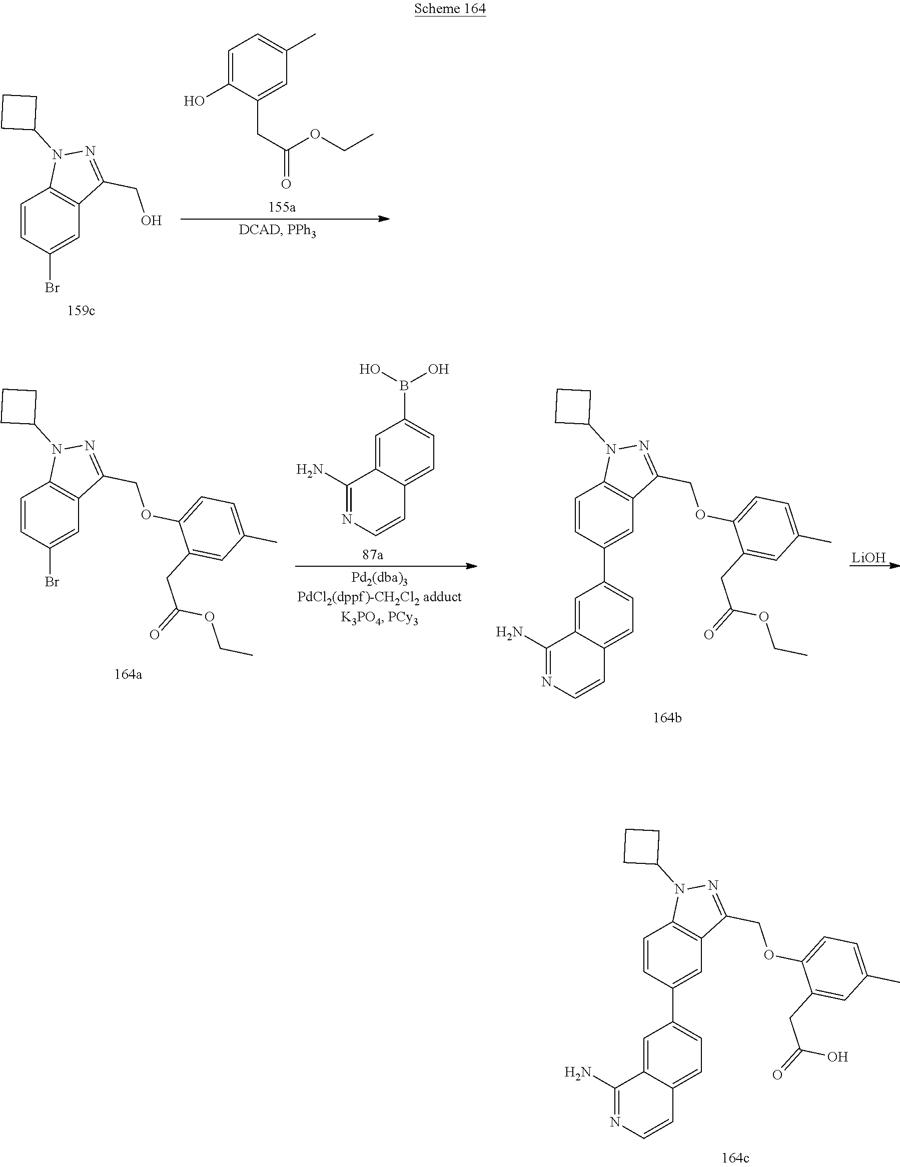

### Preparation of 2-(2-((5-(1-aminoisoquinolin-7-yl)-1-cyclobutyl-1H-indazol-3-yl)methoxy)-5-methylphenyl)acetic acid (164c)

### Step-1: Preparation ethyl 2-(2-((5-bromo-1-cyclobutyl-1H-indazol-3-yl)methoxy)-5-methylphenyl)acetate (164a)

Compound 164a was prepared according to the procedure reported in step-2 of scheme 2, from (5-bromo-1-cyclobutyl-1H-indazol-3-yl)methanol (159c) (500 mg, 1.778 mmol) in DCM (10 mL) using PPh₃ (513 mg, 1.956 mmol), ethyl 2-(2-hydroxy-5-methylphenyl)acetate (155a) (415 mg, 2.134 mmol) and a solution of DCAD (718 mg, 1.956 mmol) in DCM (10 mL) to afford after workup and purification using method-AU, ethyl 2-(2-((5-bromo-1-cyclobutyl-1H-indazol-3-yl)methoxy)-5-methylphenyl)acetate (164a) (650 mg, 80% yield) as a white solid; MS (ES+): 457.1, 459.1 (M+1).

### Step-2: Preparation ethyl 2-(2-((5-(1-aminoisoquinolin-7-yl)-1-cyclobutyl-1H-indazol-3-yl)methoxy)-5-methylphenyl)acetate (164b)

Compound 164b was prepared according to the procedure reported in step-5 of scheme 1 from ethyl 2-(2-((5-bromo-1-cyclobutyl-1H-indazol-3-yl)methoxy)-5-methylphenyl)acetate (164a) (255 mg, 0.558 mmol) in dioxane/MeTHF (10 mL, 1:1) using 1-aminoisoquinolin-7-ylboronic acid (87a) (136 mg, 0.725 mmol), K₃PO₄ (2M aqueous solution, 0.558 mL, 2.230 mmol), PCy₃ (31.3 mg, 0.112 mmol), Pd₂(dba)₃ (51.1 mg, 0.056 mmol) and PdCl₂(dppf)-CH₂Cl₂ adduct (45.5 mg, 0.056 mmol) to afford after workup and purification using method-AE, ethyl 2-(2-((5-(1-aminoisoquinolin-7-yl)-1-cyclobutyl-1H-indazol-3-yl)methoxy)-5-methylphenyl)acetate (164b) (111 mg, 38.2% yield) as a yellow solid; (ES+): 521.2 (M+1).

### Step-3: Preparation of 2-(2-((5-(1-aminoisoquinolin-7-yl)-1-cyclobutyl-1H-indazol-3-yl)methoxy)-5-methylphenyl)acetic acid (164c)

Compound 164c was prepared according to the procedure reported step-2 of scheme 1 from ethyl 2-(2-((5-(1-aminoisoquinolin-7-yl)-1-cyclobutyl-1H-indazol-3-yl)methoxy)-5-methylphenyl)acetate (164b) (111 mg, 0.213 mmol) in THF (3 mL) using a solution of LiOH·H₂O (26.8 mg, 0.640 mmol) in water (1 mL) to afford after workup and purification using method-M, 2-(2-((5-(1-aminoisoquinolin-7-yl)-1-cyclobutyl-1H-indazol-3-yl)methoxy)-5-methylphenyl)acetic acid (164c) (19.7 mg, 18.76% yield) HCl salt as a white solid; ¹H NMR (300 MHz, DMSO-d₆) δ 13.09 (s, 1H, D₂O exchangeable), 12.14 (s, 1H, D₂O) exchangeable), 9.05 (s, 2H, D₂O exchangeable), 8.93 (s, 1H), 8.40 (d, J=8.4 Hz, 1H), 8.28 (s, 1H), 8.10-7.95 (m, 2H), 7.90 (d, J=8.9 Hz, 1H), 7.69 (d, J=6.9 Hz, 1H), 7.28 (d, J=6.9 Hz, 1H), 7.19 (d, J=8.3 Hz, 1H), 7.12-7.04 (m, 1H), 7.01 (d, J=2.2 Hz, 1H), 5.47 (s, 2H), 5.43-5.28 (m, 1H), 3.49 (s, 2H), 2.79-2.60 (m, 2H), 2.60-2.52 (m, 2H), 2.23 (s, 3H), 2.02-1.83 (m, 2H); MS (ES+): 493.2 (M+1); MS (ES−): 491.1 (M−1).

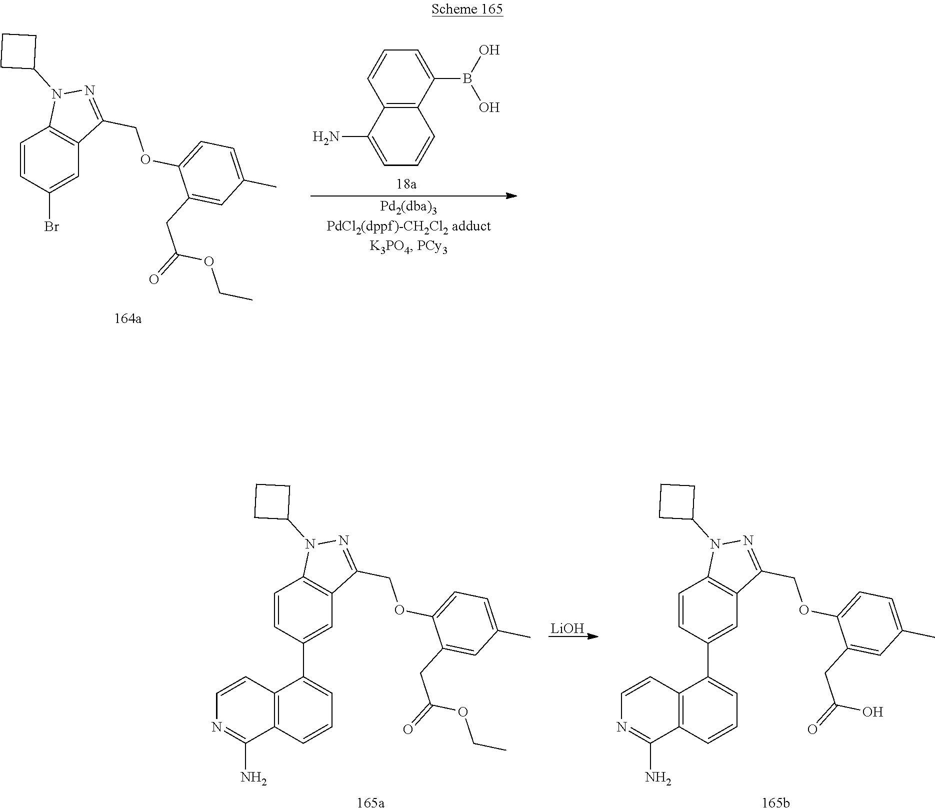

### Preparation of 2-(2-((5-(1-aminoisoquinolin-5-yl)-1-cyclobutyl-1H-indazol-3-yl)methoxy)-5-methylphenyl)acetic acid (165b)

### Step-1: Preparation ethyl 2-(2-((5-(1-aminoisoquinolin-5-yl)-1-cyclobutyl-1H-indazol-3-yl)methoxy)-5-methylphenyl)acetate (165a)

Compound 165a was prepared according to the procedure reported in step-5 of scheme 1 from ethyl 2-(2-((5-bromo-1-cyclobutyl-1H-indazol-3-yl)methoxy)-5-methylphenyl)acetate (164a) (255 mg, 0.558 mmol) in dioxane/MeTHF (10 mL, 1:1) using (1-aminoisoquinolin-5-yl)boronic acid (18a) (105 mg, 0.558 mmol), K₃PO₄ (2M aqueous solution, 0.558 mL, 2.230 mmol), PCy₃ (31.3 mg, 0.112 mmol), Pd₂(dba)₃ (51.1 mg, 0.056 mmol) and PdCl₂(dppf)-CH₂Cl₂ adduct (45.5 mg, 0.056 mmol) to afford after workup and purification using method-AE, ethyl 2-(2-((5-(1-aminoisoquinolin-5-yl)-1-cyclobutyl-1H-indazol-3-yl)methoxy)-5-methylphenyl)acetate (165a) (123 mg, 42.4% yield) as a yellow solid; (ES+): 521.3 (M+1).

### Step-2: Preparation of 2-(2-((5-(1-aminoisoquinolin-5-yl)-1-cyclobutyl-1H-indazol-3-yl)methoxy)-5-methylphenyl)acetic acid (165b)

Compound 165b was prepared according to the procedure reported step-2 of scheme 1 from ethyl 2-(2-((5-(1-aminoisoquinolin-5-yl)-1-cyclobutyl-1H-indazol-3-yl)methoxy)-5-methylphenyl)acetate (165a) (123 mg, 0.236 mmol) in THF (3 mL) using a solution of LiOH·H₂O (29.7 mg, 0.709 mmol) in water (1 mL) to afford after workup and purification using method-M, 2-(2-((5-(1-aminoisoquinolin-5-yl)-1-cyclobutyl-1H-indazol-3-yl)methoxy)-5-methylphenyl)acetic acid (165b) (17.5 mg, 15.04% yield) HCl salt as a white solid; 1H NMR (300 MHz, DMSO-d₆) δ 13.22 (s, 1H, D₂O exchangeable), 12.02 (s, 1H, D₂O exchangeable), 9.09 (s, 2H, DO exchangeable), 8.61 (d, J=8.3 Hz, 1H), 7.94 (dd, J=7.3, 1.2 Hz, 1H), 7.91-7.77 (m, 3H), 7.62 (d, J=7.2 Hz, 1H), 7.47 (dd, J=8.6, 1.7 Hz, 1H), 7.15 (d, J=8.4 Hz, 1H), 7.04 (dd, J=8.3, 2.2 Hz, 1H), 7.00-6.91 (m, 2H), 5.43 (s, 2H), 5.41-5.29 (m, 1H), 3.44 (s, 2H), 2.82-2.60 (m, 2H), 2.58-2.52 (m, 2H), 2.02-1.83 (m, 2H); MS (ES+): 493.2 (M+1); (ES−): 491.1 (M−1).

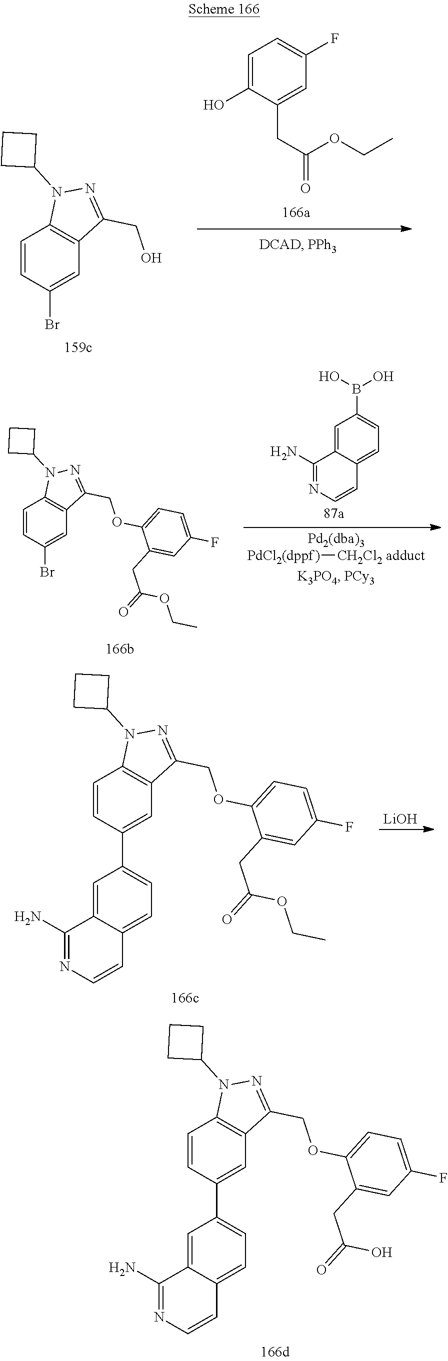

### Preparation of 2-(2-((5-(1-aminoisoquinolin-7-yl)-1-cyclobutyl-1H-indazol-3-yl)methoxy)-5-fluorophenyl)acetic acid (166d)

### Step-1: Preparation ethyl 2-(2-((5-bromo-1-cyclobutyl-1H-indazol-3-yl)methoxy)-5-fluorophenyl)acetate (166b)

Compound 166b was prepared according to the procedure reported in step-2 of scheme 2, from (5-bromo-1-cyclobutyl-1H-indazol-3-yl)methanol (159c) (500 mg, 1.778 mmol) in DCM (10 mL) using PPh₃ (513 mg, 1.956 mmol), ethyl 2-(5-fluoro-2-hydroxyphenyl)acetate (166a) (423 mg, 2.134 mmol; CAS #1261826-26-9) and a solution of DCAD (718 mg, 1.956 mmol) in DCM (10 mL) to afford after workup and purification using method-AU, ethyl 2-(2-((5-bromo-1-cyclobutyl-1H-indazol-3-yl)methoxy)-5-fluorophenyl)acetate (166b) (600 mg, 73.1% yield) as a white solid; MS (ES+): 461.0, 463.0 (M+1).

### Step-2: Preparation ethyl 2-(2-((5-(1-aminoisoquinolin-7-yl)-1-cyclobutyl-1H-indazol-3-yl)methoxy)-5-fluorophenyl)acetate (166c)

Compound 166c was prepared according to the procedure reported in step-5 of scheme 1 from ethyl 2-(2-((5-bromo-1-cyclobutyl-1H-indazol-3-yl)methoxy)-5-fluorophenyl)acetate (166b) (255 mg, 0.553 mmol) in dioxane/MeTHF (10 mL, 1:1) using 1-aminoisoquinolin-7-ylboronic acid (87a) (135 mg, 0.719 mmol), K₃PO₄ (2M aqueous solution, 0.553 mL, 2.211 mmol), PCy₃ (31 mg, 0.111 mmol), Pd₂(dba)₃ (50.6 mg, 0.055 mmol) and PdCl₂(dppf)-CH₂Cl₂ adduct (45.1 mg, 0.055 mmol) to afford after workup and purification using method-AE, ethyl 2-(2-((5-(1-aminoisoquinolin-7-yl)-1-cyclobutyl-1H-indazol-3-yl)metboxy)-5-fluorophenyl)acetate (166c) (95 mg, 32.8% yield) as a yellow solid; (ES+): 525.2 (M+1).

### Step-3: Preparation of 2-(2-((5-(1-aminoisoquinolin-7-yl)-1-cyclobutyl-1H-indazol-3-yl)methoxy)-5-fluorophenyl)acetic acid (166d)

Compound 166d was prepared according to the procedure reported step-2 of scheme 1 from ethyl 2-(2-((5-(1-aminoisoquinolin-7-yl)˜1-cyclobutyl-1H-indazol-3-yl)methoxy)-5-fluorophenyl)acetate (166c) (95 mg, 0.181 mmol) in THF (3 mL) using a solution of LiOH·H₂O (22.8 mg, 0.543 mmol) in water (1 mL) to afford after workup and purification using method-M, 2-(2-((5-(1-aminoisoquinolin-7-yl)-1-cyclobutyl-1H-indazol-3-yl)methoxy)-5-fluorophenyl)acetic acid (166d) (6.5 mg, 7.23% yield) HCl salt as a white solid; 1H NMR (300 MHz, DMSO-d₆) δ 13.12 (s, 1H, D₂O exchangeable), 12.37 (s, 1H, D₂O exchangeable), 9.03 (s, 2H, D₂O exchangeable), 8.93 (s, 1H), 8.42 (d, J=8.5 Hz, 1H), 8.30 (d, J=1.4 Hz, 1H), 8.09-7.96 (m, 2H), 7.91 (d, J=8.9 Hz, 1H), 7.69 (d, J=6.9 Hz, 1H), 7.37-7.24 (m, 2H), 7.18-7.04 (m, 2H), 5.49 (s, 2H), 5.45-5.28 (m, 1H), 3.55 (s, 2H), 2.80-2.61 (m, 2H), 2.60-2.52 (m, 2H), 2.02-1.83 (m, 2H); ¹⁹F NMR (282 MHz, DMSO de) 8-123.75; MS (ES+): 497.2 (M+1); (ES−): 495.1 (M−1).

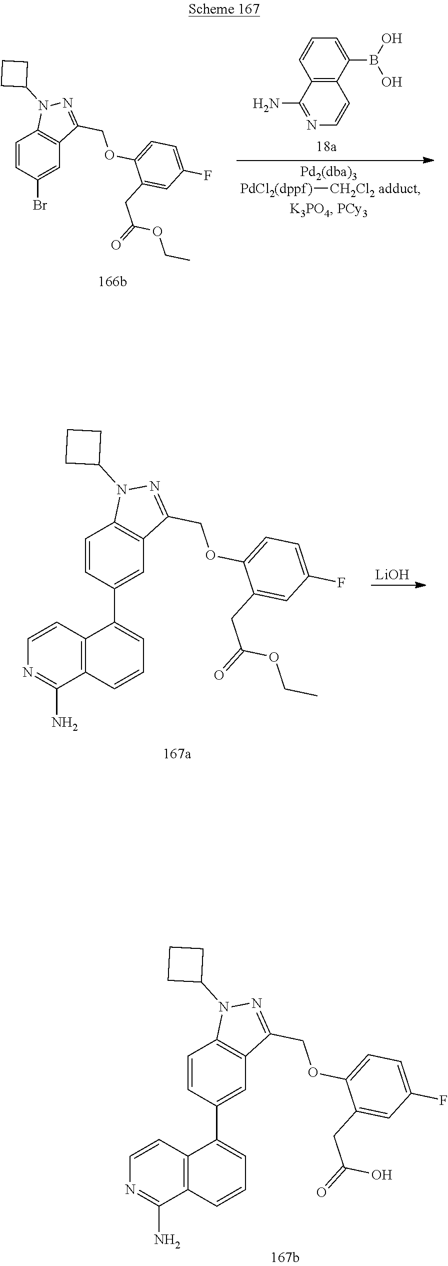

### Preparation of 2-(2-((5-(1-aminoisoquinolin-5-yl)-1-cyclobutyl-1H-indazol-3-yl)methoxy)-5-fluorophenyl)acetic acid (167b)

### Step-1: Preparation ethyl 2-(2-((5-(1-aminoisoquinolin-5-yl)-1-cyclobutyl-1H-indazol-3-yl)methoxy)-5-fluorophenyl)acetate (167a)

Compound 167a was prepared according to the procedure reported in step-5 of scheme 1 from ethyl 2-(2-((5-bromo-1-cyclobutyl-1H-indazol-3-yl)methoxy)-5-fluorophenyl)acetate (166b) (255 mg, 0.553 mmol) in dioxane/MeTHF (10 mL, 1:1) using (I-aminoisoquinolin-5-yl)boronic acid (18a) (104 mg, 0.553 mmol), K₃PO₄ (2M aqueous solution, 0.553 mL, 2.211 mmol), PCy₃ (31 mg, 0.111 mmol), Pd₂(dba)₃ (50.6 mg, 0.055 mmol) and PdCl₂(dppf)-CH₂Cl₂ adduct (45.1 mg, 0.055 mmol) to afford after workup and purification using method-AE, ethyl 2-(2-((5-(1-aminoisoquinolin-5-yl)˜1-cyclobutyl-1H-indazol-3-yl)methoxy)-5-fluorophenyl)acetate (167a) (155 mg, 53.5% yield) as a yellow solid; (ES+): 525.2 (M+1).

### Step-2: Preparation of 2-(2-((5-(1-aminoisoquinolin-5-yl)-1-cyclobutyl-1H-indazol-3-yl)methoxy)-5-fluorophenyl)acetic acid (167b)

Compound 167b was prepared according to the procedure reported step-2 of scheme 1 from ethyl 2-(2-((5-(1-aminoisoquinolin-5-yl)-1-cyclobutyl-1H-indazol-3-yl)methoxy)-5-fluorophenyl)acetate (167a) (155 mg, 0.295 mmol) in THF (3 mL) using a solution of LiOH·H₂O (37.2 mg, 0.886 mmol) in water (I mL) to afford after workup and purification using method-M, 2-(2-((5-(1-aminoisoquinolin-5-yl)-1-cyclobutyl-1H-indazol-3-yl)methoxy)-5-fluorophenyl)acetic acid (167b) (30.1 mg, 20.52% yield) HCl salt as a white solid; ¹H NMR (300 MHz, DMSO-d₆) δ 13.27 (s, 1H, D₂O exchangeable), 12.16 (s, 1H, D₂O exchangeable), 9.15 (s, 2H, D₂O exchangeable), 8.62 (d, J=8.3 Hz, 1H), 7.95 (dd, J=7.4, 1.1 Hz, 1H), 7.92-7.80 (m, 3H), 7.62 (d, J=7.2 Hz, 1H), 7.53-7.40 (m, 1H), 7.35-7.21 (m, 1H), 7.17-7.03 (m, 2H), 6.97 (d, J=7.2 Hz, 1H), 5.46 (s, 2H), 5.42-5.28 (m, 1H), 3.50 (s, 2H), 2.81-2.61 (m, 2H), 2.58-2.52 (m, 2H), 2.01-1.82 (m, 2H); ¹⁹F NMR (282 MHZ, DMSO-d₆) δ −123.70; MS (ES+): 497.2 (M+1); (ES−): 495.1 (M−1).

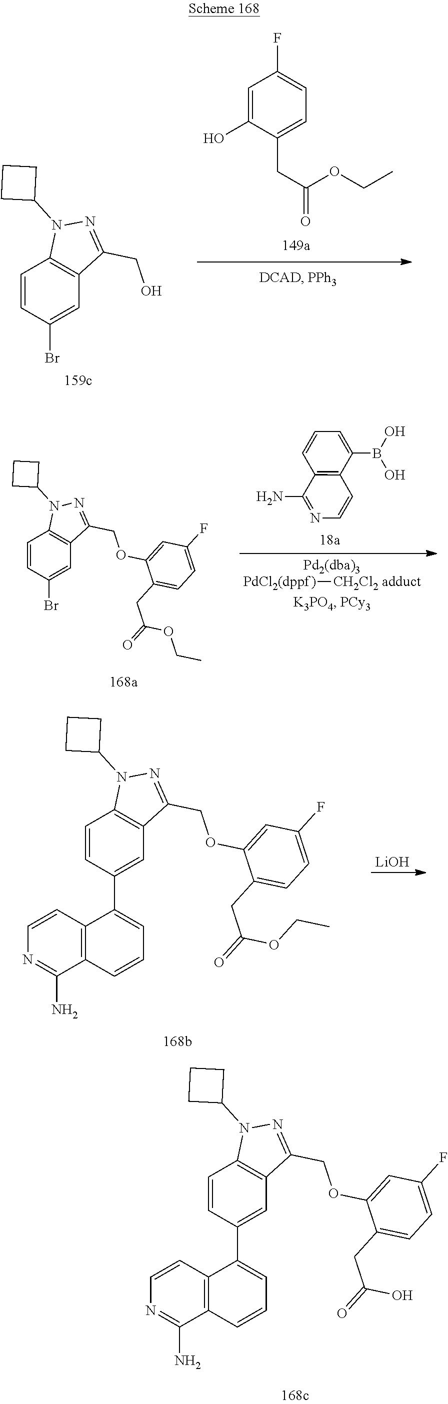

### Preparation of 2-(2-((5-(1-aminoisoquinolin-5-yl)-1-cyclobutyl-1H-indazol-3-yl)methoxy)-4-fluorophenyl)acetic acid (168c)

### Step-1: Preparation ethyl 2-(2-((5-bromo-1-cyclobutyl-1H-indazol-3-yl)methoxy)-4-fluorophenyl)acetate (168a)

Compound 168a was prepared according to the procedure reported in step-2 of scheme 2, from (5-bromo-1-cyclobutyl-1H-indazol-3-yl)methanol (159c) (400 mg, 1.423 mmol) in DCM (10 mL) using PPh₃ (410 mg, 1.565 mmol) and ethyl 2-(4-fluoro-2-hydroxyphenyl)acetate (149a) (338 mg, 1.707 mmol) and a solution of DCAD (575 mg, 1.565 mmol) in DCM (10 mL) to afford after workup and purification using method-AU, ethyl 2-(2-((5-bromo-1-cyclobutyl-1H-indazol-3-yl)methoxy)-4-fluorophenyl)acetate (168a) (245 mg, 0.531 mmol, 37.3% yield) as a white solid; MS (ES+): 461.0, 463.0 (M+1).

### Step-2: Preparation of ethyl 2-(2-((5-(1-aminoisoquinolin-5-yl)-1-cyclobutyl-1H-indazol-3-yl)methoxy)-4-fluorophenyl)acetate (168b)

Compound 168b was prepared according to the procedure reported in step-5 of scheme 1 from ethyl 2-(2-((5-bromo-1-cyclobutyl-1H-indazol-3-yl)methoxy)-4-fluorophenyl)acetate (168a) (110 mg, 0.238 mmol) in dioxane/MeTHF (10 mL, 1:1) using (1-aminoisoquinolin-5-yl)boronic acid (18a) (44.8 mg, 0.238 mmol), K₃PO₄ (2M aqueous solution, 0.238 mL, 0.954 mmol), PCy₃ (13.37 mg, 0.048 mmol), Pd₂(dba)₃ (21.83 mg, 0.024 mmol) and PdCl₂(dppf)-CH₂Cl₂ adduct (19.47 mg, 0.024 mmol) to afford after workup and purification using method-AE, ethyl 2-(2-((5-(1-aminoisoquinolin-5-yl)-1-cyclobutyl-1H-indazol-3-yl)methoxy)-4-fluorophenyl)acetate (168b) (77 mg, 61.6% yield) as a yellow solid; MS (ES+): 525.2 (M+1).

### Step-3: Preparation of 2-(2-((5-(1-aminoisoquinolin-5-yl)-1-cyclobutyl-1H-indazol-3-yl)methoxy)-4-fluorophenyl)acetic acid (168c)

Compound 168c was prepared according to the procedure reported step-2 of scheme 1 from ethyl 2-(2-((5-(1-aminoisoquinolin-5-yl)-1-cyclobutyl-1H-indazol-3-yl)methoxy)-4-fluorophenyl)acetate (168b) (77 mg, 0.147 mmol) in THF (3 mL) using a solution of LiOH·H₂O (18.48 mg, 0.44 mmol) in water (1 mL) to afford after workup and purification using method-M, 2-(2-((5-(1-aminoisoquinolin-5-yl)-1-cyclobutyl-1H-indazol-3-yl)methoxy)-4-fluorophenyl)acetic acid (168c) (20.0 mg, 27.4% yield) HCl salt as a white solid; 1H NMR (300 MHz, DMSO-d₆) δ 12.97 (s, 1H, D₂O exchangeable), 12.04 (s, 1H, D₂O exchangeable), 8.96 (s, 2H, D₂O exchangeable), 8.57 (d, J=8.1 Hz, 1H), 7.94 (d, J=7.3 Hz, 1H), 7.91-7.81 (m, 3H), 7.60 (d, J=7.2 Hz, 1H), 7.47 (dd, J=8.9, 1.5 Hz, 1H), 7.28-7.14 (m, 2H), 6.98 (d, J=7.3 Hz, 1H), 6.73 (td, J=8.4, 2.5 Hz, 1H), 5.49 (s, 2H), 5.46-5.26 (m, 1H), 3.45 (s, 2H), 2.81-2.60 (m, 2H), 2.54-2.46 (m, 2H), 1.99-1.85 (m, 2H); ¹⁹F NMR (282 MHz, DMSO-d₆) δ −113.00; MS (ES+): 497.2 (M+1); (ES−): 495.2 (M−1).

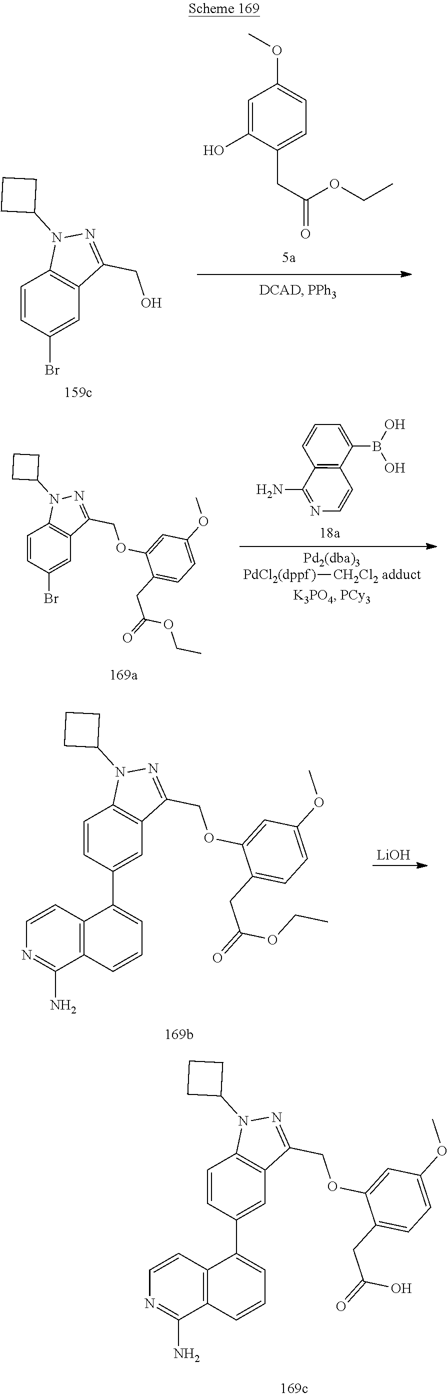

### Preparation of 2-(2-((5-(1-aminoisoquinolin-5-yl)-1-cyclobutyl-1H-indazol-3-yl)methoxy)-4-methoxyphenyl)acetic acid (169c)

### Step-1: Preparation ethyl 2-(2-((5-bromo-1-cyclobutyl-1H-indazol-3-yl)methoxy)-4-methoxyphenyl)acetate (169a)

Compound 169a was prepared according to the procedure reported in step-2 of scheme 2, from (5-bromo-1-cyclobutyl-1H-indazol-3-yl)methanol (159c) (400 mg, 1.423 mmol), in DCM (10 mL) using PPh₃ (410 mg, 1.565 mmol) and ethyl 2-(2-hydroxy-4-methoxyphenyl)acetate (5a) (359 mg, 1.707 mmol) and a solution of DCAD (575 mg, 1.565 mmol) in DCM (10 mL) to afford after workup and purification using method-AU, ethyl 2-(2-((5-bromo-1-cyclobutyl-1H-indazol-3-yl)methoxy)-4-methoxyphenyl)acetate (169a) (395 mg, 58.7% yield) as a white solid; MS (ES+): 473.1, 475.1 (M+1).

### Step-2: Preparation of ethyl 2-(2-((5-(1-aminoisoquinolin-5-yl)-1-cyclobutyl-1H-indazol-3-yl)methoxy)-4-methoxyphenyl)acetate (169b)

Compound 169b was prepared according to the procedure reported in step-5 of scheme 1 from ethyl 2-(2-((5-bromo-1-cyclobutyl-1H-indazol-3-yl)methoxy)-4-methoxyphenyl)acetate (169a) (188 mg, 0.397 mmol) in dioxane/Me THF (10 mL, 1:1) using (1-aminoisoquinolin-5-yl)boronic acid (18a) (74.7 mg, 0.397 mmol), K₃PO₄ (2M aqueous solution, 0.397 mL, 1.589 mmol), PCy₃ (22.28 mg, 0.079 mmol), Pd₂(dba)₃ (36.4 mg, 0.04 mmol) and PdCl₂(dppf)-CH₂Cl₂ adduct (32.4 mg, 0.04 mmol) to afford after workup and purification using method-AE, ethyl 2-(2-((5-(1-aminoisoquinolin-5-yl)-1-cyclobutyl-1H-indazol-3-yl)methoxy)-4-methoxyphenyl)acetate (169b) (90 mg, 42.2% yield) as a yellow solid; (ES+): 537.3 (M+1).

### Step-3: Preparation of 2-(2-((5-(1-aminoisoquinolin-5-yl)-1-cyclobutyl-1H-indazol-3-yl)methoxy)-4-methoxyphenyl)acetic acid (169c)

Compound 169c was prepared according to the procedure reported step-2 of scheme 1 from ethyl 2-(2-((5-(1-aminoisoquinolin-5-yl)-1-cyclobutyl-1H-indazol-3-yl)methoxy)-4-methoxyphenyl)acetate (169b) (90 mg, 0.168 mmol) in THF (3 mL) using a solution of LiOH·H₂O (21.11 mg, 0.503 mmol) in water (1 mL) to afford after workup and purification using method-M, 2-(2-((5-(1-aminoisoquinolin-5-yl)-1-cyclobutyl-1H-indazol-3-yl)methoxy)-4-methoxyphenyl)acetic acid (169c) (22.7 mg, 26.6% yield) HCl salt as a white solid; 1H NMR (300 MHz, DMSO-d₆) δ 12.98 (s, 1H, D₂O exchangeable), 11.93 (s, 1H, D₂O exchangeable), 9.03 (s, 2H, D₂O exchangeable), 8.58 (d, J=8.3 Hz, 1H), 7.94 (d, J=7.2 Hz, 1H), 7.89-7.81 (m, 3H), 7.60 (d, J=7.3 Hz, 1H), 7.46 (dd, J=8.8, 1.5 Hz, 1H), 7.06 (d, J=8.3 Hz, 1H), 6.98 (d, J=7.3 Hz, 1H), 6.87 (d, J=2.4 Hz, 1H), 6.47 (dd, J=8.4, 2.4 Hz, 1H), 5.47 (s, 2H), 5.42-5.33 (m, 1H), 3.74 (s, 3H), 3.39 (s, 2H), 2.74-2.61 (m, 2H), 2.58-2.42 (m, 2H), 1.99-1.86 (m, 2H); MS (ES+): 509.2 (M+1); (ES−): 507.1 (M−1).

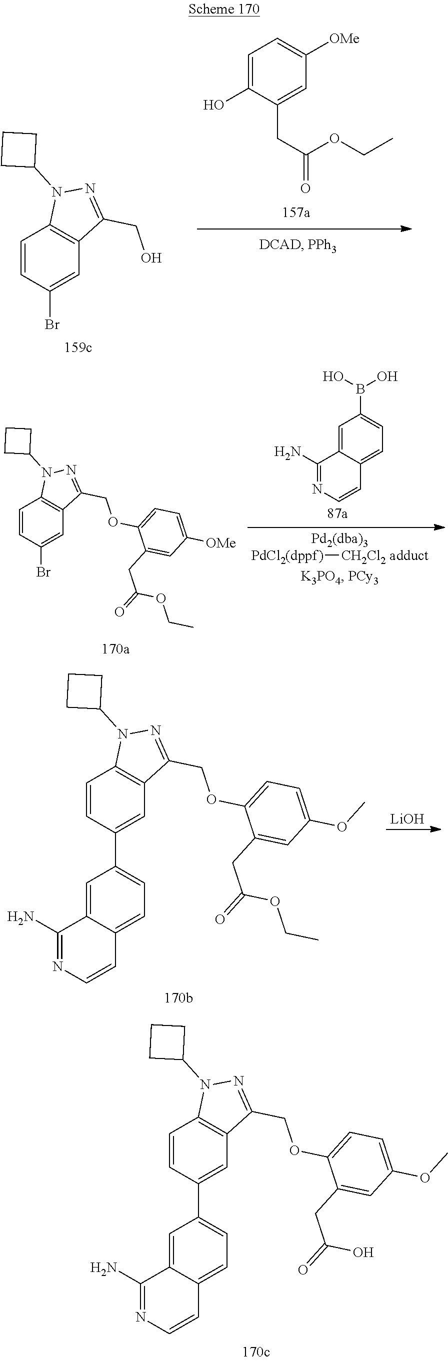

### Preparation of 2-(2-((5-(1-aminoisoquinolin-7-yl)-1-cyclobutyl-1H-indazol-3-yl)methoxy)-5-methoxyphenyl)acetic acid (170c)

### Step-1: Preparation of ethyl 2-(2-((5-bromo-1-cyclobutyl-1H-indazol-3-yl)methoxy)-5-methoxyphenyl)acetate (170a)

Compound 170a was prepared according to the procedure reported in step-2 of scheme 2, from (5-bromo-1-cyclobutyl-1H-indazol-3-yl)methanol (159c) (400 mg, 1.423 mmol), in DCM (10 mL) using PPh₃ (410 mg, 1.565 mmol) and ethyl 2-(2-hydroxy-5-methoxyphenyl)acetate (157a) (359 mg, 1.707 mmol) and a solution of DCAD (575 mg, 1.565 mmol) in DCM (10 mL) to afford after workup and purification using method-AU, ethyl 2-(2-((5-bromo-1-cyclobutyl-1H-indazol-3-yl)methoxy)-5-methoxyphenyl)acetate (170a) (403 mg, 59.8% yield) as a white solid; MS (ES+): 473.0, 475.0 (M+1).

### Step-2: Preparation of ethyl 2-(2-((5-(1-aminoisoquinolin-7-yl)-1-cyclobutyl-1H-indazol-3-yl)methoxy)-5-methoxyphenyl)acetate (170b)

Compound 170b was prepared according to the procedure reported in step-5 of scheme 1 from ethyl 2-(2-((5-bromo-1-cyclobutyl-1H-indazol-3-yl)methoxy)-5-methoxyphenyl)acetate (170a) (192 mg, 0.406 mmol) in dioxane/MeTHF (10 mL, 1:1) using 1-aminoisoquinolin-7-ylboronic acid (87a) (99.5 mg, 0.53 mmol), K₃PO₄ (2M aqueous solution, 0.406 mL, 1.622 mmol), PCy₃ (22.75 mg, 0.081 mmol), Pd₂(dba)₃ (37.1 mg, 0.041 mmol) and PdCl₂(dppf)-CH₂Cl₂ adduct (33.1 mg, 0.041 mmol) to afford after workup and purification using method-AE, ethyl 2-(2-((5-(1-aminoisoquinolin-7-yl)-1-cyclobutyl-1H-indazol-3-yl)methoxy)-5-methoxyphenyl)acetate (170b) (71 mg, 32.6% yield) as a yellow solid; (ES+): 537.3 (M+1).

### Step-3: Preparation of 2-(2-((5-(1-aminoisoquinolin-7-yl)-1-cyclobutyl-1H-indazol-3-yl)methoxy)-5-methoxyphenyl)acetic acid (170c)

Compound 170c was prepared according to the procedure reported step-2 of scheme 1 from ethyl 2-(2-((5-(1-aminoisoquinolin-7-yl)-1-cyclobutyl-1H-indazol-3-yl)methoxy)-5-methoxyphenyl)acetate (170b) (71 mg, 0.132 mmol) in THF (3 mL) using a solution of LiOH·H₂O (16.66 mg, 0.397 mmol) in water (1 mL) to afford after workup and purification using method-M, 2-(2-((5-(1-aminoisoquinolin-7-yl)-1-cyclobutyl-1H-indazol-3-yl)methoxy)-5-methoxyphenyl)acetic acid (170c) (11.9 mg, 17.69% yield) HCl salt as a light tan solid; 1H NMR (300 MHz, DMSO-d₆) δ 13.06 (s, 1H, D₂O exchangeable), 12.14 (s, 1H, D₂O exchangeable), 9.01 (s, 2H, D₂O exchangeable), 8.92 (s, 1H), 8.40 (d, J=8.3 Hz, 1H). 8.28 (s, 1H), 8.07-7.96 (m, 2H), 7.90 (d, J=8.8 Hz, 1H), 7.69 (d, J=6.7 Hz, 1H), 7.28 (d, J=6.9 Hz, 1H), 7.22 (d, J=8.8 Hz, 1H), 6.89-6.84 (m, 1H), 6.82 (s, 1H), 5.44 (s, 2H), 5.41-5.33 (m, 1H), 3.70 (s, 3H), 3.52 (s, 2H), 2.79-2.54 (m, 4H), 2.00-1.82 (m, 2H); MS (ES+): 509.2 (M+1); (ES−): 507.1 (M−1).

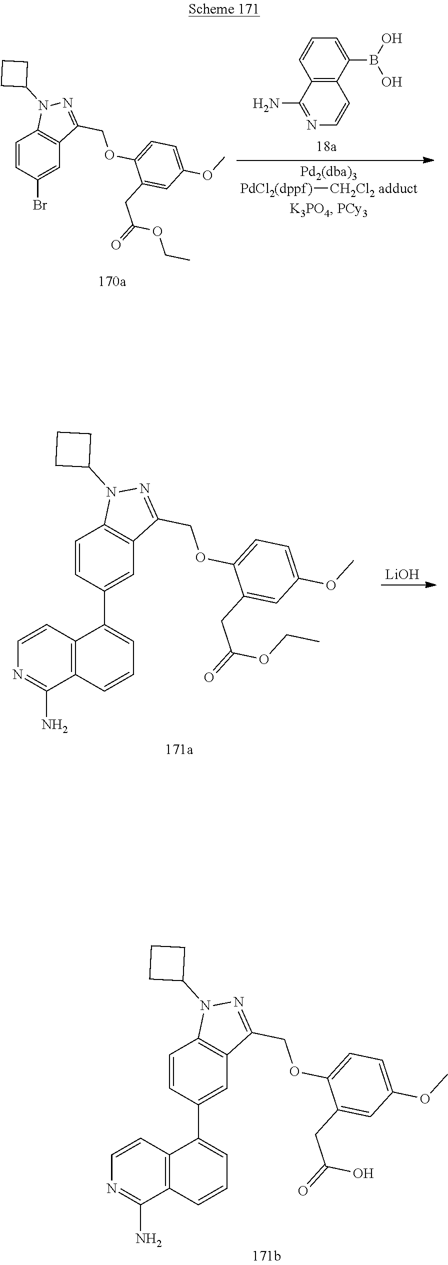

### Preparation of 2-(2-((5-(1-aminoisoquinolin-5-yl)-1-cyclobutyl-1H-indazol-3-yl)methoxy)-5-methoxyphenyl)acetic acid (171b)

### Step-1: Preparation of ethyl 2-(2-((5-(1-aminoisoquinolin-5-yl)-1-cyclobutyl-1H-indazol-3-yl)methoxy)-5-methoxyphenyl)acetate (171a)

Compound 171a was prepared according to the procedure reported in step-2 of scheme 2, from ethyl 2-(2-((5-bromo-1-cyclobutyl-1H-indazol-3-yl)methoxy)-5-methoxyphenyl)acetate (170a) (192 mg, 0.406 mmol) in dioxane/MeTHF (10 mL, 1:1) using (1-aminoisoquinolin-5-yl)boronic acid (18a) (76 mg, 0.406 mmol), K₃PO₄ (2M aqueous solution, 0.406 mL, 1.622 mmol), PCy₃ (22.75 mg, 0.081 mmol), Pd₂(dba)₃ (37.1 mg, 0.041 mmol) and PdCl₂(dppf)-CH₂Cl₂ adduct (33.1 mg, 0.041 mmol) to afford after workup and purification using method-AE, ethyl 2-(2-((5-(1-aminoisoquinolin-5-yl)-1-cyclobutyl-1H-indazol-3-yl)methoxy)-5-methoxyphenyl)acetate (171a) (80 mg, 36.8% yield) as a yellow solid; (ES+): 537.3 (M+1).

### Step-2: Preparation of 2-(2-((5-(1-aminoisoquinolin-5-yl)-1-cyclobutyl-1H-indazol-3-yl)methoxy)-5-methoxyphenyl)acetic acid (171b)

Compound 171b was prepared according to the procedure reported step-2 of scheme 1 from ethyl 2-(2-((5-(1-aminoisoquinolin-5-yl)-1-cyclobutyl-1H-indazol-3-yl)methoxy)-5-methoxyphenyl)acetate (171a) (80 mg, 0.149 mmol) in THF (3 mL) using a solution of LiOH·H₂O (18.77 mg, 0.447 mmol) in water (1 mL) to afford after workup and purification using method-M, 2-(2-((5-(1-aminoisoquinolin-5-yl)-1-cyclobutyl-1H-indazol-3-yl)methoxy)-5-methoxyphenyl)acetic acid (171b) (19.2 mg, 25.3% yield) HCl salt as a white solid; 1H NMR (300 MHz, DMSO-d₆) δ 13.03 (s, 1H, D₂O exchangeable), 12.03 (s, 1H, D₂O exchangeable), 9.03 (s, 2H, D₂O exchangeable), 8.59 (d, J=8.3 Hz, 1H), 7.99-7.91 (m, 1H), 7.91-7.81 (m, 3H), 7.61 (d, J=7.3 Hz, 1H), 7.46 (dd, J=8.7, 1.7 Hz, 1H), 7.18 (dd, 1H), 6.97 (d, J=7.3 Hz, 1H), 6.85-6.74 (m, 2H), 5.40 (s, 2H), 5.38-5.33 (m, 1H), 3.68 (s, 3H), 3.45 (s, 2H), 2.78-2.61 (m, 2H), 2.59-2.43 (m, 2H), 2.01-1.83 (m, 2H); MS (ES+): 509.2 (M+1); (ES−): 507.1 (M−1).

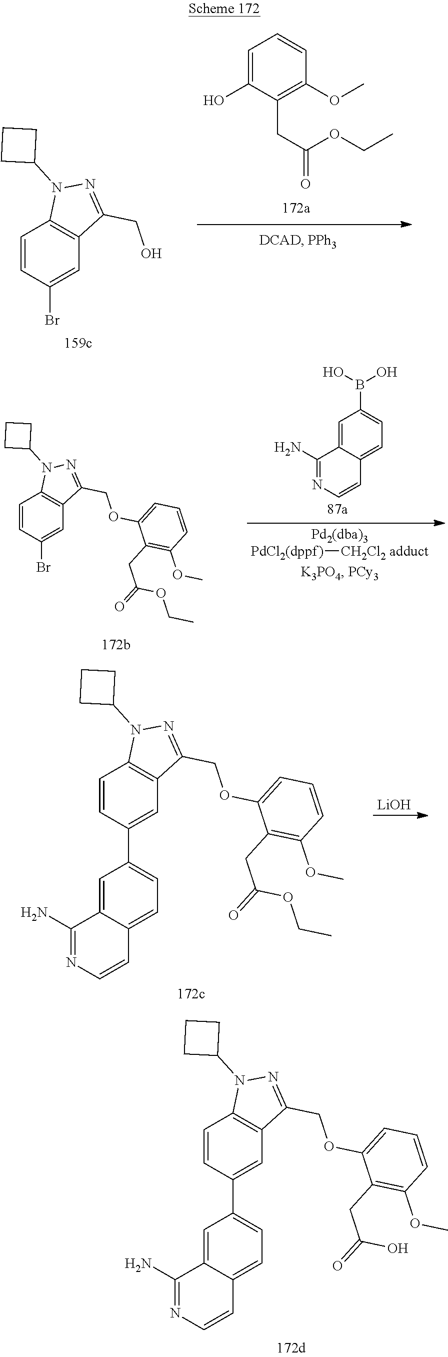

### Preparation of 2-(2-((5-(1-aminoisoquinolin-7-yl)-1-cyclobutyl-1H-indazol-3-yl)methoxy)-6-methoxyphenyl)acetic acid (172d)

### Step-1: Preparation of ethyl 2-(2-((5-bromo-1-cyclobutyl-1H-indazol-3-yl)methoxy)-6-methoxyphenyl)acetate (172b)

Compound 172b was prepared according to the procedure reported in step-2 of scheme 2, from (5-bromo-1-cyclobutyl-1H-indazol-3-yl)methanol (159c) (535 mg, 1.903 mmol) in DCM (10 mL) using PPh₃ (549 mg, 2.093 mmol), ethyl 2-(2-hydroxy-6-methoxyphenyl)acetate (172a) (440 mg, 2.093 mmol; CAS #187968-46-3) and a solution of DCAD (769 mg, 2.093 mmol) in DCM (10 mL) to afford after workup and purification using method-AU, ethyl 2-(2-((5-bromo-1-cyclobutyl-1H-indazol-3-yl)methoxy)-6-methoxyphenyl)acetate (172b) (320 mg, 35.5% yield) as a white solid; MS (ES+): 473.1, 475.1 (M+1).

### Step-2: Preparation of ethyl 2-(2-((5-(1-aminoisoquinolin-7-yl)-1-cyclobutyl-1H-indazol-3-yl)methoxy)-6-methoxyphenyl)acetate (172c)

Compound 172c was prepared according to the procedure reported in step-5 of scheme 1 from ethyl 2-(2-((5-bromo-1-cyclobutyl-1H-indazol-3-yl)methoxy)-6-methoxyphenyl)acetate (172b) (145 mg, 0.306 mmol) in dioxane/MeTHF (10 mL, 1:1) using 1-aminoisoquinolin-7-ylboronic acid (87a) (74.7 mg, 0.398 mmol), K₃PO₄ (2M aqueous solution, 0.306 mL, 1.225 mmol), PCy₃ (17.18 mg, 0.061 mmol), Pd₂(dba)₃ (28.1 mg, 0.031 mmol) and PdCl₂(dppf)-CH₂Cl₂ adduct (25.02 mg, 0.031 mmol) to afford after workup and purification using method-AE, ethyl 2-(2-((5-(1-aminoisoquinolin-7-yl)-1-cyclobutyl-1H-indazol-3-yl)methoxy)-6-methoxyphenyl)acetate (172c) (110 mg, 66.9% yield) as a yellow solid; MS (ES+): 537.2 (M+1).

### Step-3: Preparation of 2-(2-((5-(1-aminoisoquinolin-7-yl)-1-cyclobutyl-1H-indazol-3-yl)methoxy)-6-methoxyphenyl)acetic acid (172d)

Compound 172d was prepared according to the procedure reported step-2 of scheme 1 from ethyl 2-(2-((5-(1-aminoisoquinolin-7-yl)-1-cyclobutyl-1H-indazol-3-yl)methoxy)-6-methoxyphenyl)acetate (172c) (110 mg, 0.205 mmol) in THF (3 mL) using a solution of LiOH·H₂O (25.8 mg, 0.615 mmol) in water (1 mL) to afford after workup and purification using method-M, 2-(2-((5-(1-aminoisoquinolin-7-yl)-1-cyclobutyl-1H-indazol-3-yl)methoxy)-6-methoxyphenyl)acetic acid (172d) (56.8 mg, 54.5% yield) HCl salt as a light tan solid; 1H NMR (300 MHz, DMSO-d₆) δ 13.14 (s, 1H, D₂O exchangeable), 11.98 (s, 1H, D₂O exchangeable), 9.08 (s, 2H, D₂O) exchangeable), 8.91 (s, 1H), 8.37 (dd, J=8.5, 1.6 Hz, 1H), 8.26 (d, 1H), 8.06-7.95 (m, 2H), 7.88 (d, J=8.9 Hz, 1H), 7.67 (d, J=6.9 Hz, 1H), 7.29-7.23 (m, 2H), 6.95 (d, J=8.3 Hz, 1H), 6.66 (d, J=8.3 Hz, 1H), 5.48 (s, 2H), 5.44-5.25 (m, 1H), 3.72 (s, 3H), 3.47 (s, 2H), 2.78-2.56 (m, 2H), 2.52-2.45 (m, 2H), 2.00-1.81 (m, 2H); MS (ES+): 509.2 (M+1); (ES−): 507.1 (M−1).

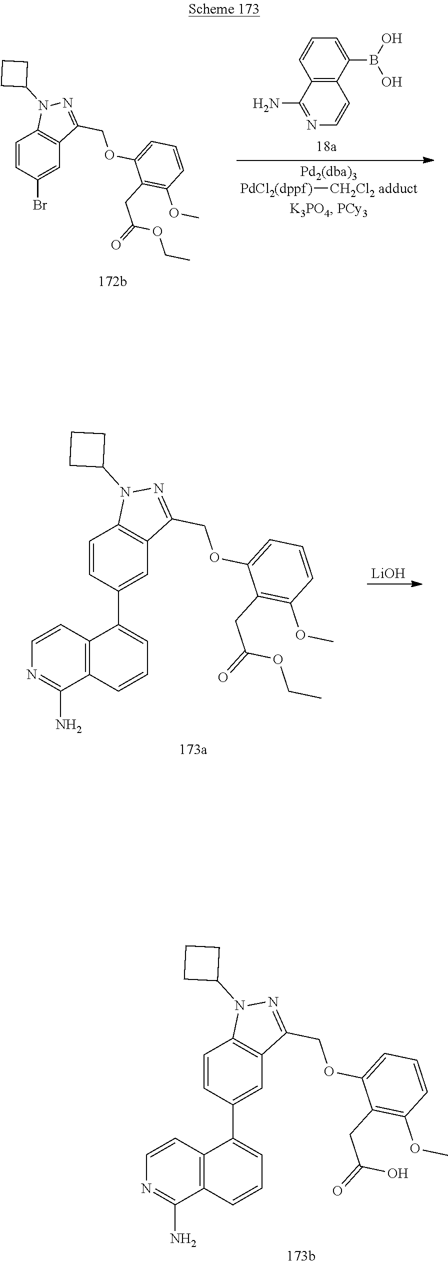

### Preparation of 2-(2-((5-(1-aminoisoquinolin-5-yl)-1-cyclobutyl-1H-indazol-3-yl)methoxy)-6-methoxyphenyl)acetic acid (173b)

### Step-1: Preparation of ethyl 2-(2-((5-(1-aminoisoquinolin-5-yl)-1-cyclobutyl-1H-indazol-3-yl)methoxy)-6-methoxyphenyl)acetate (173a)

Compound 173a was prepared according to the procedure reported in step-5 of scheme 1 from ethyl 2-(2-((5-bromo-1-cyclobutyl-1H-indazol-3-yl)methoxy)-6-methoxyphenyl)acetate (172b) (145 mg, 0.306 mmol) in dioxane/MeTHF (10 mL, 1:1) using (I-aminoisoquinolin-5-yl)boronic acid (18a) (74.7 mg, 0.397 mmol), K₃PO₄ (2M aqueous solution, 0.306 mL, 1.225 mmol), PCy₃ (17.18 mg, 0.061 mmol), Pd₂(dba)₃ (28.1 mg, 0.031 mmol) and PdCl₂(dppf)-CH₂Cl₂ adduct (25.02 mg, 0.031 mmol) to afford after workup and purification using method-AE, ethyl 2-(2-((5-(1-aminoisoquinolin-5-yl)-1-cyclobutyl-1H-indazol-3-yl)methoxy)-6-methoxyphenyl)acetate (173a) (75 mg, 45.6% yield) as a yellow solid; (ES+): 537.2 (M+1).

### Step-2: Preparation of 2-(2-((5-(1-aminoisoquinolin-5-yl)-1-cyclobutyl-1H-indazol-3-yl)methoxy)-6-methoxyphenyl)acetic acid (173b)

Compound 173b was prepared according to the procedure reported step-2 of scheme 1 from ethyl 2-(2-((5-(1-aminoisoquinolin-5-yl)-1-cyclobutyl-1H-indazol-3-yl)methoxy)-6-methoxyphenyl)acetate (173a) (75 mg, 0.147 mmol) in THF (3 mL) using a solution of LiOH·H₂O (18.57 mg, 0.442 mmol) in water (I mL)) to afford after workup and purification using method-M, 2-(2-((5-(1-aminoisoquinolin-5-yl)-1-cyclobutyl-1H-indazol-3-yl)methoxy)-6-methoxyphenyl)acetic acid (173b) (18.6 mg, 24.8% yield) HCl salt as a white solid; ¹H NMR (300 MHz, DMSO-d₆) δ 13.10 (s, 1H, D₂O exchangeable), 11.89 (s, 1H, D₂O exchangeable), 9.07 (s, 2H, D₂O exchangeable), 8.59 (d, J=8.2 Hz, 1H), 7.95-7.90 (m, 1H), 7.90-7.80 (m, 3H), 7.61 (d, J=7.2 Hz, 1H), 7.46 (dd, J=8.7, 1.6 Hz, 1H), 7.22 (t, J=8.3 Hz, 1H), 6.94 (dd, J=12.0, 7.8 Hz, 2H), 6.65 (d, J=8.3 Hz, 1H), 5.45 (s, 2H), 5.42-5.30 (m, 1H), 3.72 (s, 3H), 3.44 (s, 2H), 2.78-2.62 (m, 2H), 2.54-2.46 (m, 2H), 2.00-1.84 (m, 2H); MS (ES+): 509.2 (M+1); (ES−): 507.1 (M−1).

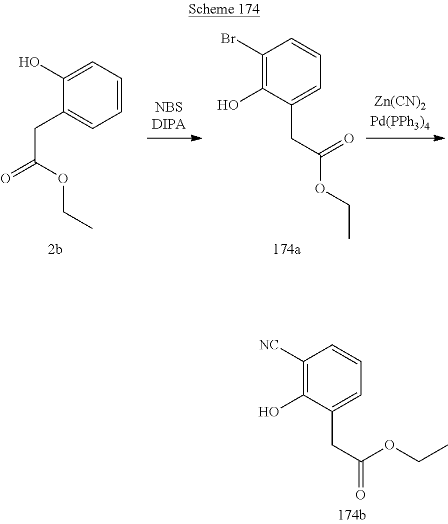

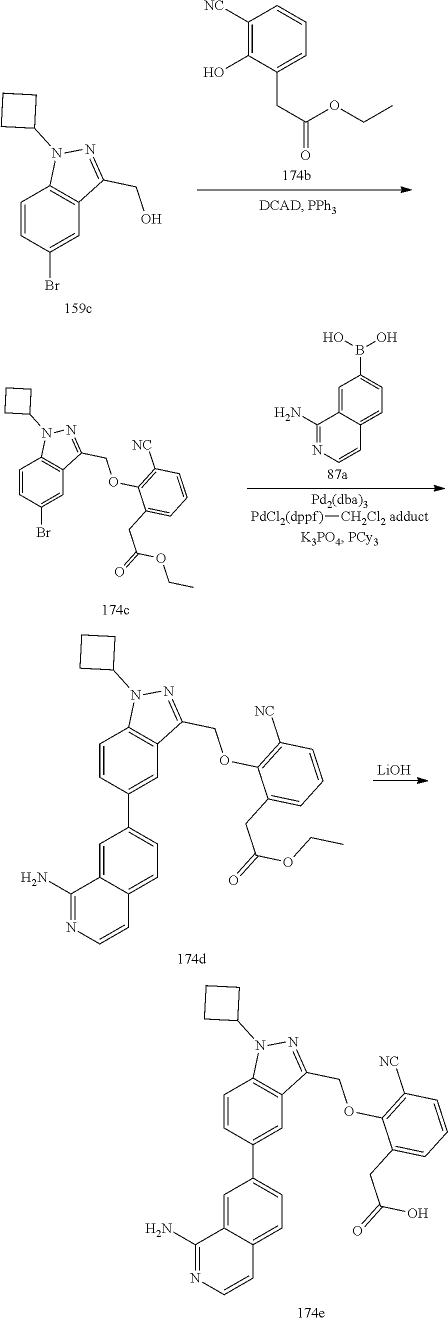

### Preparation of 2-(2-((5-(1-aminoisoquinolin-7-yl)-1-cyclobutyl-1H-indazol-3-yl)methoxy)-3-cyanophenyl)acetic acid (174e)

### Step-1: Preparation of ethyl 2-(3-bromo-2-hydroxyphenyl)acetate (174a)

To a stirred solution of ethyl 2-(2-hydroxyphenyl)acetate (2b) (6.0 g, 33.29 mmol) in DCM (150 mL) was added diisopropylamine (DIPA, 3.78 g, 37.36 mmol), followed by N-bromosuccinimide (NBS) (portion wise) (2.06 g, 11.57 mmol) at 0° C. and stirred at RT for 1 h. The mixture was poured into water (100, mL) and extracted with ethyl acetate (2×250 mL). Combined organics were washed with brine (100 mL), dried, filtered and concentrated in vacuum. The residue obtained was purified using method-AY to give ethyl 2-(3-bromo-2-hydroxyphenyl)acetate (174a) (5.0 g, 57.9%) as a white solid; 1H NMR (300 MHz, DMSO-de) δ 9.84 (s, 1H), 7.37-7.18 (m, 2H), 6.75 (d, J=8.6 Hz, 1H), 4.06 (q, J=7.1 Hz, 2H), 3.55 (s, 2H), 1.17 (t, J=7.1 Hz, 3H).

### Step-2: Preparation of ethyl 2-(3-cyano-2-hydroxyphenyl)acetate (174b)

Compound 174b was prepared according to the procedure reported in step-1 of scheme 108 from ethyl 2-(3-bromo-2-hydroxyphenyl)acetate (174a) (35 g, 135.08 mmol) in DMF (350 mL) using Zn(CN)₂ (15.86 g, 135.08 mmol), Pd(PPb₃)₄ (15.61 g, 13.51 mmol) and heating at 120° C. for 16 h. This gave after workup and purification using method-AY, ethyl 2-(3-cyano-2-hydroxyphenyl)acetate (174b) (11.1 g, 40% yield) as a white solid; 1H NMR (300 MHz, DMSO-d₆) δ 10.42 (s, 1H), 7.54 (dd, J=7.7, 1.7 Hz, 1H), 7.45 (dd, J=7.6, 1.7 Hz, 1H), 6.95 (t, J=7.6 Hz, 1H), 4.08 (q, J=7.1 Hz, 2H), 3.71 (s, 2H), 1.19 (t, J=7.1 Hz, 3H); ES (MS+): 206.1 (M+1).

### Step-3: Preparation of ethyl 2-(2-((5-bromo-1-cyclobutyl-1H-indazol-3-yl)methoxy)-3-cyanophenyl)acetate (174c)

Compound 174c was prepared according to the procedure reported in step-2 of scheme 2, from (5-bromo-1-cyclobutyl-1H-indazol-3-yl)methanol (159c) (2.5 g, 8.89 mmol) in DCM (20 mL) using PPh₃ (2.57 g, 9.78 mmol), ethyl 2-(3-cyano-2-hydroxyphenyl)acetate (174b) (2.190 g, 10.67 mmol) and a solution of DCAD (3.59 g, 9.78 mmol) in DCM (20 mL) to afford after workup and purification using method-AU, ethyl 2-(2-((5-bromo-1-cyclobutyl-1H-indazol-3-yl)methoxy)-3-cyanophenyl)acetate (174c) (3.1 g, 74.4% yield) as a white solid; MS (ES+): 490.0, 492.1 (M+Na).

### Step-4: Preparation of ethyl 2-(2-((5-(1-aminoisoquinolin-7-yl)-1-cyclobutyl-1H-indazol-3-yl)methoxy)-3-cyanophenyl)acetate (174d)

Compound 174d was prepared according to the procedure reported in step-5 of scheme 1 from ethyl 2-(2-((5-bromo-1-cyclobutyl-1H-indazol-3-yl)methoxy)-3-cyanophenyl)acetate (174c) (1.5 g, 3.20 mmol) in dioxane/MeTHF (15 mL, 1:1) using 1-aminoisoquinolin-7-ylboronic acid (87a) (0.783 g, 4.16 mmol), K₃PO₄ (2M aqueous solution, 3.20 mL, 12.81 mmol), PCy₃ (0.180 g, 0.641 mmol). Pd₂(dba)₃ (0.293 g, 0.320 mmol) and PdCl₂(dppf)-CH₂Cl₂ adduct (0.262 g, 0.320 mmol) to afford after workup and purification using method-AE, ethyl 2-(2-((5-(1-aminoisoquinolin-7-yl)-1-cyclobutyl-1H-indazol-3-yl)methoxy)-3-cyanophenyl)acetate (174d) (605 mg, 35.5% yield) as a yellow solid; MS (ES+): 532.1 (M−1).

### Step-5: Preparation of 2-(2-((5-(1-aminoisoquinolin-7-yl)-1-cyclobutyl-1H-indazol-3-yl)methoxy)-3-cyanophenyl)acetic acid (174e)

Compound 174e was prepared according to the procedure reported step-2 of scheme 1 from ethyl 2-(2-((5-(1-aminoisoquinolin-7-yl)-1-cyclobutyl-1H-indazol-3-yl)methoxy)-3-cyanophenyl)acetate (174d) (0.605 mg, 1.14 mmol) in THF (3 mL) using a solution of LiOH·H₂O (87.5 mg, 3.44 mmol) in water (1 mL) to afford after workup and purification using method-M, 2-(2-((5-(1-aminoisoquinolin-7-yl)-1-cyclobutyl-1H-indazol-3-yl)methoxy)-3-cyanophenyl)acetic acid (174e) (72.3 mg, 0.144 mmol) HCl salt as a white solid; ¹H NMR (300 MHz, DMSO-d₆) δ 12.97 (s, 1H, D₂O exchangeable), 9.12 (s, 2H, D₂O exchangeable), 8.96 (s, 1H), 8.40-8.28 (m, 2H), 8.11-7.99 (m, 2H), 7.91 (d, J=8.9 Hz, 1H), 7.82-7.61 (m, 3H), 7.35-7.22 (m, 2H), 5.56 (s, 2H), 5.45-5.28 (m, 1H), 3.67 (s, 2H), 2.70-2.58 (m, 2H), 2.52-2.51 (m, 2H), 1.98-1.82 (m, 2H); MS (ES+): 504.2 (M+1); (ES−): 502.1 (M−1).

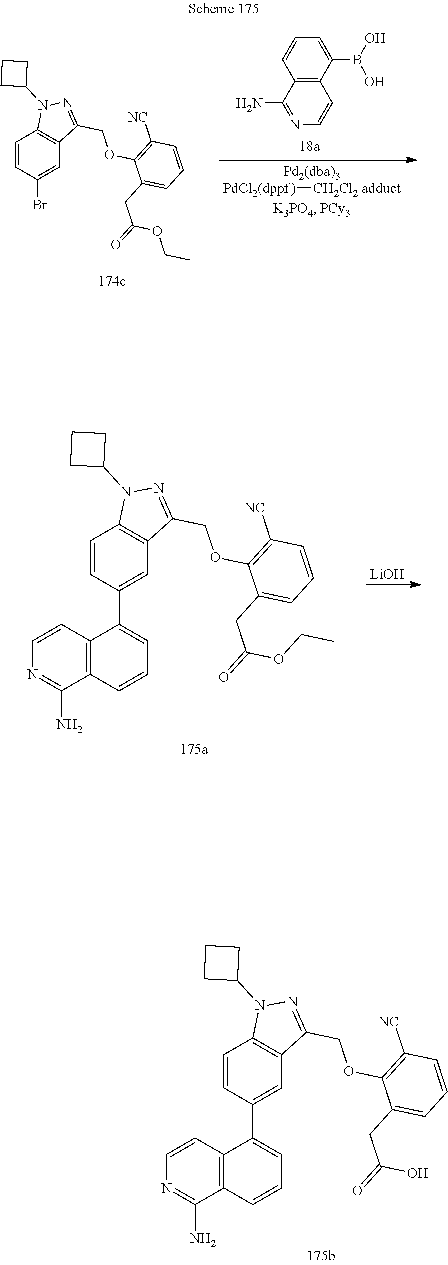

### Preparation of 2-(2-((5-(1-aminoisoquinolin-5-yl)-1-cyclobutyl-1H-indazol-3-yl)methoxy)-3-cyanophenyl)acetic acid (175b)

### Step-1: Preparation of ethyl 2-(2-((5-(1-aminoisoquinolin-5-yl)-1-cyclobutyl-1H-indazol-3-yl)methoxy)-3-cyanophenyl)acetate (175a)

Compound 175a was prepared according to the procedure reported in step-5 of scheme 1 from ethyl 2-(2-((5-bromo-1-cyclobutyl-1H-indazol-3-yl)methoxy)-3-cyanophenyl)acetate (174c) (1.5 g, 3.20 mmol) in dioxane/MeTHF (15 mL, 1:1) using (I-aminoisoquinolin-5-yl)boronic acid (18a) (0.602 g, 3.2 mmol), K₃PO₄ (2M aqueous solution, 3.20 mL, 12.81 mmol), PCy₃ (0.180 g, 0.641 mmol), Pd₂(dba)₃ (0.293 g, 0.320 mmol) and PdCl₂(dppf)-CH₂Cl₂ adduct (0.262 g, 0.320 mmol) to afford after workup and purification using method-AE, ethyl 2-(2-((5-(1-aminoisoquinolin-5-yl)-1-cyclobutyl-1H-indazol-3-yl)methoxy)-3-cyanophenyl)acetate (175a) (337 mg, 19.79% yield) as a yellow solid; (ES+): 532.2 (M+1).

### Step-2: Preparation of 2-(2-((5-(1-aminoisoquinolin-5-yl)-1-cyclobutyl-1H-indazol-3-yl)methoxy)-3-cyanophenyl)acetic acid (175b)

Compound 175b was prepared according to the procedure reported step-2 of scheme 1 from ethyl 2-(2-((5-(1-aminoisoquinolin-5-yl)-1-cyclobutyl-1H-indazol-3-yl)methoxy)-3-cyanophenyl)acetate (175a) (0.337 mg, 0.634 mmol) in THF (3 mL) using a solution of LiOH·H₂O (47.34 mg, 1.902 mmol) in water (1 mL) to afford after workup and purification using method-M, 2-(2-((5-(1-aminoisoquinolin-5-yl)-1-cyclobutyl-1H-indazol-3-yl)methoxy)-3-cyanophenyl)acetic acid (175b) (52.7 mg, 0.105 mmol) HCl salt as a white solid; ¹H NMR (300 MHz, DMSO-d₆) δ 13.20 (s, 1H, D₂O exchangeable), 12.38 (s, 1H, D₂O exchangeable), 9.06 (s, 2H, D₂O exchangeable), 8.64-8.52 (m, 1H), 7.86-7.75 (m, 4H), 7.65 (dd, J=7.7, 1.7 Hz, 1H), 7.60-7.52 (m, 2H), 7.39 (dd, J=8.6, 1.7 Hz, 1H), 7.21 (t, J=7.7 Hz, 1H), 6.86 (d, J=7.2 Hz, 1H), 5.45 (s, 2H), 5.41-5.23 (m, 1H), 3.53 (s, 2H), 2.70-2.50 (m, 2H), 2.47-2.40 (m, 2H), 1.95-1.73 (m, 2H); MS (ES+): 504.2 (M+1).

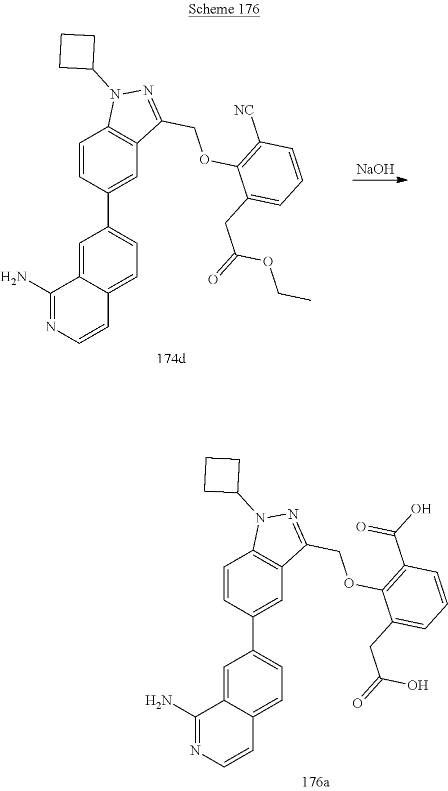

### Preparation of 2-((5-(1-aminoisoquinolin-7-yl)-1-cyclobutyl-1H-indazol-3-yl)methoxy)-3-(carboxymethyl)benzoic acid (176a)

To a solution of ethyl 2-(2-((5-(1-aminoisoquinolin-7-yl)-1-cyclobutyl-1H-indazol-3-yl)methoxy)-3-cyanophenyl)acetate (174d) (200 mg, 0.376 mmol) in ethanol (10 mL) was added NaOH (376 mg, 9.41 mmol) and heated at reflux for 18 h. The reaction mixture was diluted with water (20 mL) and acidified with conc. HCl. The solid separated was filtered and dried to give 2-((5-(1-aminoisoquinolin-7-yl)-1-cyclobutyl-1H-indazol-3-yl)methoxy)-3-(carboxymethyl)benzoic acid (176a) (11.9 mg, 6.05% yield) HCl salt as a white solid; 1H NMR (300 MHz, DMSO-d₆) δ 9.01 (s, 2H, D₂O exchangeable), 8.93 (s, 1H), 8.51-8.35 (m, 2H), 8.09-7.93 (m, 3H, 1H D₂O exchangeable), 7.89 (d, J=8.9 Hz, 1H), 7.75 (s, 1H, DO exchangeable), 7.73-7.64 (m, 1H), 7.51 (dd, J=7.6, 1.8 Hz, 1H), 7.43-7.30 (m, 1H), 7.30-7.23 (m, 1H), 7.18 (t, J=7.6 Hz, 1H), 5.37 (s, 2H), 5.45-5.25 (m, 1H), 3.65 (s, 2H), 2.75-2.59 (m, 2H), 2.54-2.46 (m, 2H), 1.99-1.84 (m, 2H); MS (ES+): 523.2 (M+1).

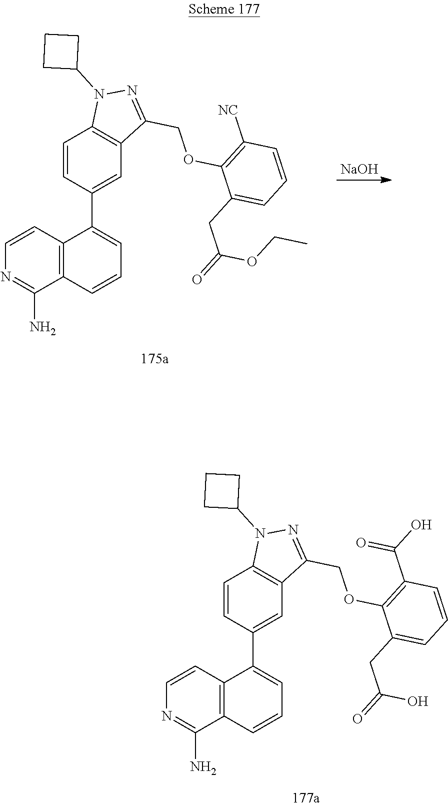

### Preparation of 2-((5-(1-aminoisoquinolin-5-yl)-1-cyclobutyl-1H-indazol-3-yl)methoxy)-3-(carboxymethyl)benzoic acid (177a)

Compound 177a was prepared according to the procedure reported in scheme 176 from ethyl 2-(2-((5-(1-aminoisoquinolin-5-yl)-1-cyclobutyl-1H-indazol-3-yl)methoxy)-3-cyanophenyl)acetate (175a) (300 mg, 0.564 mmol) in ethanol (10 mL) using NaOH (0.705 mL, 14.11 mmol) to afford after workup 2-((5-(1-aminoisoquinolin-5-yl)-1-cyclobutyl-1H-indazol-3-yl)methoxy)-3-(carboxymethyl)benzoic acid (177a) (52.8 mg, 18% yield) HCl salt as a white solid; ¹H NMR (300 MHz, DMSO-d₆) δ 13.26 (s, 1H, D₂O exchangeable), 12.26 (s, 1H, D₂O exchangeable), 9.06 (s, 2H, D₂O) exchangeable), 8.61 (d, J=8.1 Hz, 1H), 7.95-7.79 (m, 5H, 1H D₂O exchangeable), 7.67-7.60 (m, 1H), 7.60-7.54 (m, 1H, D₂O exchangeable), 7.50-7.43 (m, 2H), 7.39-7.32 (m, 1H), 7.16 (t, J=7.6 Hz, 1H), 6.99-6.93 (m, 1H), 5.45-5.36 (m, 1H), 5.34 (s, 2H), 3.58 (s, 2H), 2.80-2.61 (m, 2H), 2.55-2.52 (m, 2H), 2.01-1.81 (m, 2H); MS (ES+): 523.2 (M+1).

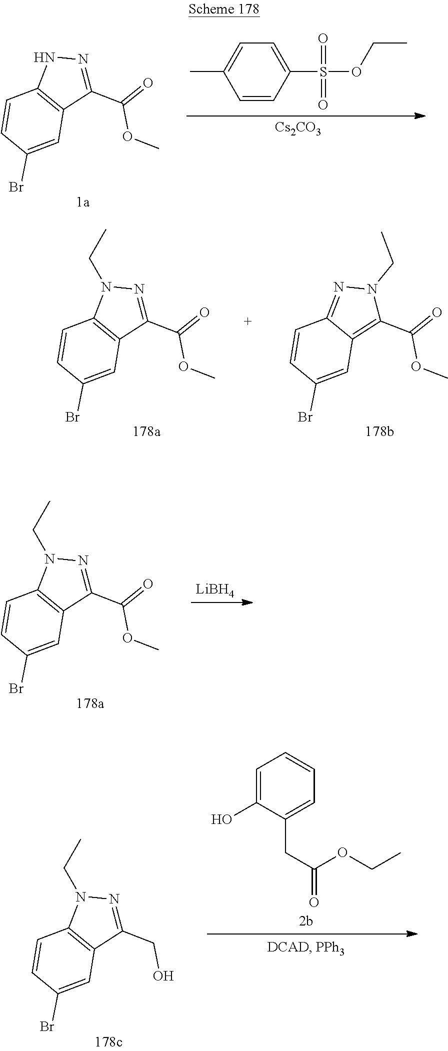

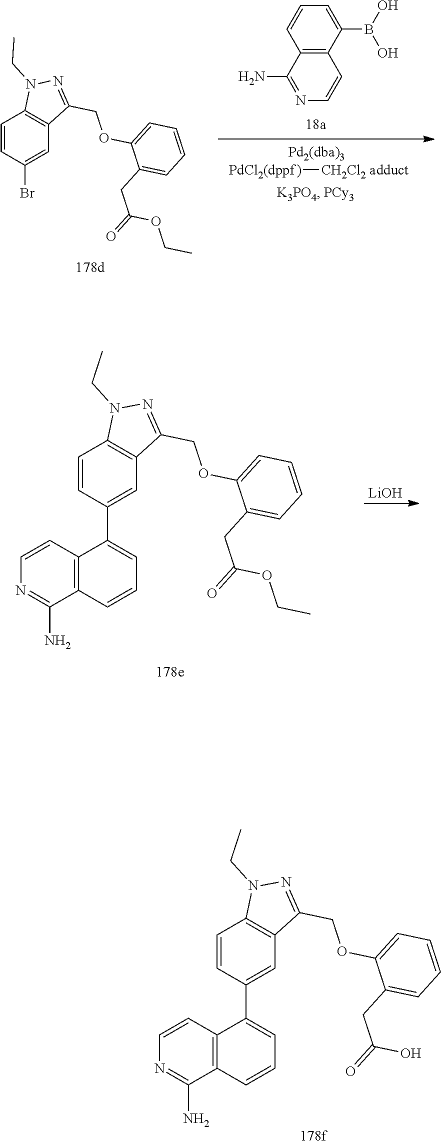

### Preparation of 2-(2-((5-(1-aminoisoquinolin-5-yl)-1-ethyl-1H-indazol-3-yl)methoxy)phenyl)acetic acid (178f)

### Step 1: Preparation of methyl 5-bromo-1-ethyl-1H-indazole-3-carboxylate (178a) and methyl 5-bromo-2-ethyl-2H-indazole-3-carboxylate (178b)

Compounds 178a and 178b were prepared according to the procedure reported in step-2 of scheme 86 from methyl 5-bromo-1H-indazole-3-carboxylate (1a) (1.274 g, 4.99 mmol) in DMF (20 mL) using Cs₂CO₃ (3.25 g, 9.99 mmol) and ethyl 4-methylbenzenesulfonate (1 g, 4.99 mmol; CAS #80-40-0) to afford after workup and purification using method-A, methyl 5-bromo-1-ethyl-1H-indazole-3-carboxylate (178a) (700 mg, 49.5% yield) as a yellow oil; JH NMR (300 MHz, DMSO-d₆) δ 8.20 (dd, J=1.9, 0.7 Hz, 1H), 7.86 (dd, J=9.0, 0.7 Hz, 1H), 7.63 (dd, J=8.9, 1.9 Hz, 1H), 4.56 (q, J=7.2 Hz, 2H), 3.93 (s, 3H), 1.43 (t, J=7.2 Hz, 3H); MS (ES+): 283.00 & 285.00 (M+1) and methyl 5-bromo-2-ethyl-2H-indazole-3-carboxylate (178b) (450 mg, 31.8% yield) as a yellow solid; 1H NMR (300 MHz, DMSO-d₆) δ 8.15 (dd, J=1.9, 0.7 Hz, 1H), 7.79 (dd, J=9.1, 0.8 Hz, 1H), 7.49 (dd, J=9.1, 1.9 Hz, 1H), 4.85 (q, J=7.2 Hz, 2H), 3.99 (s, 3H), 1.47 (t, J=7.2 Hz, 3H); MS (ES+): 283.00 & 285.00 (M+1).

### Step 2: Preparation of (5-bromo-1-ethyl-1H-indazol-3-yl)methanol (178c)

Compound 178c was prepared according to the procedure reported in step-2 of scheme 119 from methyl 5-bromo-1-ethyl-1H-indazole-3-carboxylate (178a) (1.780 g, 6.29 mmol) in THF (25 mL) using LiBH₄ (7.86 mL, 15.72 mmol) and MeOH (0.636 mL, 15.72 mmol) to afford after workup and purification using method-AW, (5-bromo-1-ethyl-1H-indazol-3-yl)methanol (178c) (1.335 g, 83% yield) as a clear oil; MS (ES+): 255.0/257.0 M+1).

### Step 3: Preparation of ethyl 2-(2-((5-bromo-1-ethyl-1H-indazol-3-yl)methoxy)phenyl)acetate (178d)

Compound 178d was prepared according to the procedure reported in step-2 of scheme 2, from (5-bromo-1-ethyl-1H-indazol-3-yl)methanol (178c) (1.300 g, 5.10 mmol) in DCM (25 mL) using PPh₃ (1.470 g, 5.61 mmol), ethyl 2-(2-hydroxyphenyl)acetate (2b) (1.102 g, 6.11 mmol) and a solution of DCAD (2.058 g, 5.61 mmol) in DCM to afford after workup and purification using method-AU, ethyl 2-(2-((5-bromo-1-ethyl-1H-indazol-3-yl)methoxy)phenyl)acetate (178d) (1.062 g, 49.9% yield) as a white solid; MS (ES+): 417.0/419.0 (M+1).

### Step 4: Preparation of ethyl 2-(2-((5-(1-aminoisoquinolin-5-yl)-1-ethyl-1H-indazol-3-yl)methoxy)phenyl)acetate (178e)

Compound 178e was prepared according to the procedure reported in step-5 of scheme 1 from ethyl 2-(2-((5-bromo-1-ethyl-1H-indazol-3-yl)methoxy)phenyl)acetate (178d) (0.3 g, 0.719 mmol) in dioxane/MeTHF (25 mL, 9:1) using (1-aminoisoquinolin-5-yl)boronic acid (18a) (0.135 g, 0.719 mmol). K₃PO₄ (2M aqueous solution, 0.719 mL, 2.88 mmol). PCy₃ (0.040 g, 0.144 mmol), Pd₂(dba)₃ (0.066 g, 0.072 mmol) and PdCl₂(dppf)-CH₂Cl₂ adduct (0.059 g, 0.072 mmol) to afford after workup and purification using method-AE, ethyl 2-(2-((5-(1-aminoisoquinolin-5-yl)-1-ethyl-1H-indazol-3-yl)methoxy)phenyl)acetate (178e) (112 mg, 32.4% yield) as a yellow solid; MS (ES+): 481.3 (M+1).

### Step 5: Preparation of 2-(2-((5-(1-aminoisoquinolin-5-yl)-1-ethyl-1H-indazol-3-yl)methoxy)phenyl)acetic acid (178f)

Compound 178f was prepared according to the procedure reported step-2 of scheme 1 from ethyl 2-(2-((5-(1-aminoisoquinolin-5-yl)-1-ethyl-1H-indazol-3-yl)methoxy)phenyl)acetate (178e) (112 mg, 0.233 mmol) in THF (3 mL) using a solution of LiOH·H₂O (29.3 mg, 0.699 mmol) in water (1 mL) to afford after workup and purification using method-M, 2-(2-((5-(1-aminoisoquinolin-5-yl)-1-ethyl-1H-indazol-3-yl)methoxy)phenyl)acetic acid (178f) (4.1 mg, 3.89% yield) HCl salt as a white solid; 1H NMR (300 MHz, DMSO-d₆) δ 13.15 (s, 1H, D₂O exchangeable), 12.03 (s, 1H, D₂O exchangeable), 9.05-8.99 (m, 2H, D₂O exchangeable), 8.62-8.53 (m, 1H), 7.99-7.91 (m, 1H), 7.90-7.84 (m, 3H), 7.62 (d, J=7.2 Hz, 1H), 7.48 (d, J=8.6 Hz, 1H), 7.25 (d, J=4.1 Hz, 2H), 7.18 (d, J=7.3 Hz, 1H), 6.99 (d, J=7.2 Hz, 1H), 6.94-6.88 (m, 1H), 5.45 (s, 2H), 4.53 (q. J=7.2 Hz, 2H), 3.48 (s, 2H), 1.46 (t, J=7.1 Hz, 3H); MS (ES+): 453.2 (M+1) (ES−): 451.1 (M−1).

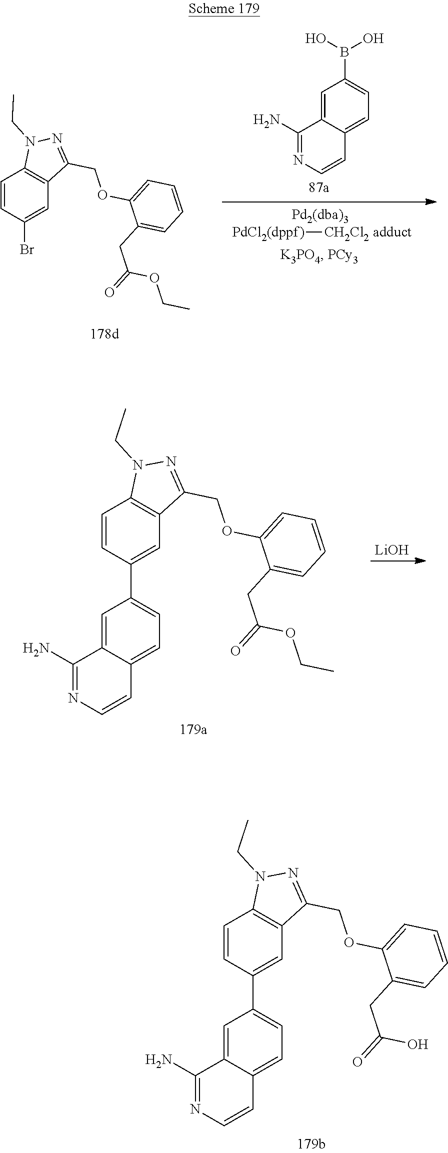

### Preparation of 2-(2-((5-(1-aminoisoquinolin-7-yl)-1-ethyl-1H-indazol-3-yl)methoxy)phenyl)acetic acid (179b)

### Step 1: Preparation of ethyl 2-(2-((5-(1-aminoisoquinolin-7-yl)-1-ethyl-1H-indazol-3-yl)methoxy)phenyl)acetate (179a)

Compound 179a was prepared according to the procedure reported in step-5 of scheme 1 from ethyl 2-(2-((5-bromo-1-ethyl-1H-indazol-3-yl)methoxy)phenyl)acetate (178d) (300 mg, 0.719 mmol) in dioxane/MeTHF (10 mL, 1:1) using 1-aminoisoquinolin-7-ylboronic acid (87a) (176 mg, 0.935 mmol), K₃PO₄ (2M aqueous solution, 0.719 ml, 2.88 mmol), PCy₃ (40 mg, 0.144 mmol), Pd₂(dba)₃ (0.066 g, 0.072 mmol) and PdCl₂(dppf)-CH₂Cl₂ adduct (59 mg, 0.072 mmol) to afford after workup and purification using method-AE, ethyl 2-(2-((5-(1-aminoisoquinolin-7-yl)-1-ethyl-1H-indazol-3-yl)methoxy)phenyl)acetate (179a) (107 mg, 31.0% yield) as a yellow solid; MS (ES+): 481.3 (M+1).

### Step 2: Preparation of 2-(2-((5-(1-aminoisoquinolin-7-yl)-1-ethyl-1H-indazol-3-yl)methoxy)phenyl)acetic acid (179b)

Compound 179b was prepared according to the procedure reported step-2 of scheme 1 from ethyl 2-(2-((5-(1-aminoisoquinolin-7-yl)-1-ethyl-1H-indazol-3-yl)methoxy)phenyl)acetate (179a) (107 mg, 0.223 mmol) in THF (3 mL) using a solution of LiOH·H₂O (28.0 mg, 0.668 mmol) in water (1 mL) to afford after workup and purification using method-M, 2-(2-((5-(1-aminoisoquinolin-7-yl)-1-ethyl-1H-indazol-3-yl)methoxy)phenyl)acetic acid (179b) (11.9 mg, 11.81% yield) HCl salt as a white solid; ¹H NMR (300 MHz, DMSO-d₆) δ 13.11 (s, 1H, D₂O exchangeable), 12.15 (s, 1H, D₂O exchangeable), 9.06 (s, 2H, D₂O exchangeable), 8.93 (d, 1H), 8.40 (dd, J=8.5, 1.7 Hz, 1H), 8.30 (d, J=1.6 Hz, 1H), 8.06-8.00 (m, 1H), 7.98 (d, J=1.7 Hz, 1H), 7.89 (d, J=8.9 Hz, 1H), 7.69 (d, J=6.9 Hz, 1H), 7.32-7.23 (m, 3H), 7.20 (d, 1H), 6.96-6.89 (m, 1H), 5.48 (s, 2H), 4.51 (q, J=7.1 Hz, 2H), 3.53 (s, 2H), 1.45 (t, J=7.2 Hz, 3H); MS (ES+): 453.2 (M+1); (ES−): 451.1 (M−1).

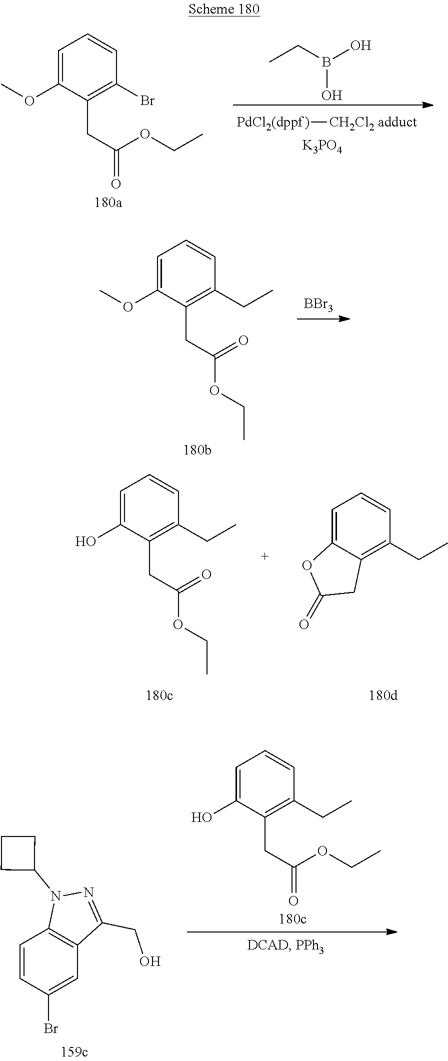

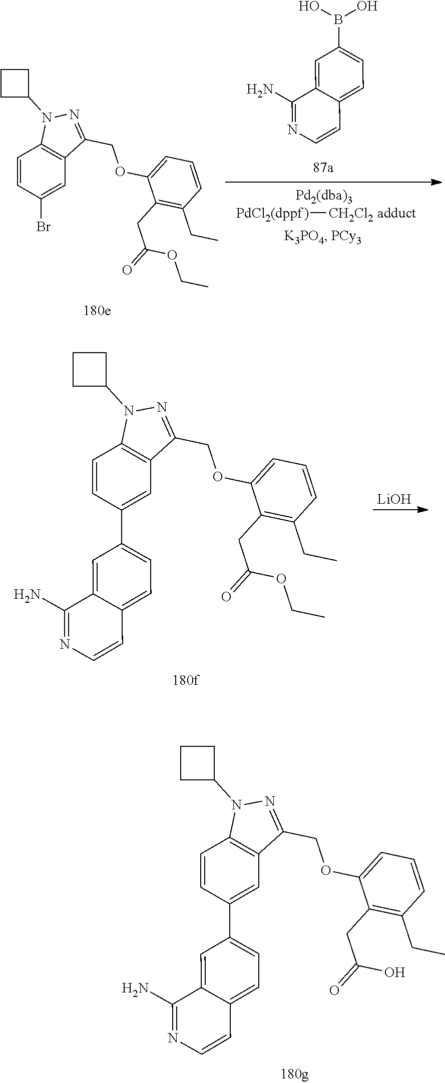

### Preparation of 2-(2-((5-(1-aminoisoquinolin-7-yl)-1-cyclobutyl-1H-indazol-3-yl)methoxy)-6-ethylphenyl)acetic acid (180g)

### Step-1: Preparation of ethyl 2-(2-ethyl-6-methoxyphenyl)acetate (180b)

To a solution of ethyl 2-(2-bromo-6-methoxyphenyl)acetate (180a) (18 g, 65.90 mmol; CAS #1261618-62-5) in 1,4-dioxane (162 mL) was added at RT ethyl boronic acid (10.95 g, 148.28 mmol), a solution of K₃PO₄ (41.96 g, 197.71 mmol) in water (50 mL), purged with N₂ gas for 15 minutes, added PdCl₂(dppf)-CH₂Cl₂ adduct (1.07 g, 1.32 mmol) and heated at 100° C. for 16 h. The reaction mixture was filtered through celite bed and the filtrate was extracted with EtOAc. The combined organic layers were washed with brine, dried, filtered and concentrated. The residue obtained was purified using method-AX, to give ethyl 2-(2-ethyl-6-methoxyphenyl)acetate (180b) (12 g, 82% yield) as a yellow liquid; 1H NMR (300 MHz. Chloroform-d) δ 7.31-7.16 (m, 1H), 6.91-6.84 (m, 1H), 6.77 (d, J=8.2 Hz, 1H), 4.17 (qd, J=7.1, 0.8 Hz, 2H), 3.83 (d, J=0.8 Hz, 3H), 3.72 (s, 2H), 2.65 (q, J=7.6 Hz, 2H), 1.31-1.16 (m, 6H).

### Step-2: Preparation of ethyl 2-(2-ethyl-6-hydroxyphenyl)acetate (180c) and 4-ethylbenzofuran-2(3H)-one (180d)

Compounds 180c and 180d were prepared according to the procedure reported in step-1 of scheme 94 from ethyl 2-(2-ethyl-6-methoxyphenyl)acetate (180b) (11 g, 49.43 mmol) in DCM (165 mL) using BBr₃ (12.38 g, 49.43 mmol) to afford after workup and purification using method-BA, ethyl 2-(2-ethyl-6-hydroxyphenyl)acetate (180c) (2 g, 19.4% yield) and 4-ethylbenzofuran-2(3H)-one (180d) (5.6 g, undesired compound). Undesired compound 4-ethylbenzofuran-2(3H)-one (180d) (5.6 g) was dissolved in ethanol (56 mL), added H₂SO₄ (1 mL) and heated at reflux for 1 h. The reaction mixture was cooled to room temperature, concentrated in vacuum and residue dissolved in EtOAc, washed with 10% NaHCO₃ solution, brine, dried, filtered and concentrated. The crude product was purified using method-BA to give additional ethyl 2-(2-ethyl-6-hydroxyphenyl)acetate (180c) (1.7 g, 16.5% yield) as a dark brown liquid; 1H NMR (300 MHz, Chloroform-d) & 7.11 (t, J=7.8 Hz, 2H), 6.82 (s, 1H), 6.79 (s, 1H), 4.20 (qd, J=7.1, 0.9 Hz, 2H), 3.72 (s, 2H), 2.68 (q, J=7.6 Hz, 2H), 1.28 (td, J=7.1, 0.9 Hz, 3H), 1.21 (td, J=7.6, 0.9 Hz, 3H); MS (ES+): 209.3 (M+1); (MS−): 207.3 (M−1).

### Step-3: Preparation of ethyl 2-(2-((5-bromo-1-cyclobutyl-1H-indazol-3-yl)methoxy)-6-ethylphenyl)acetate (180e)

Compound 180e was prepared according to the procedure reported in step-2 of scheme 2, from (5-bromo-1-cyclobutyl-1H-indazol-3-yl)methanol (159c) (1 g, 3.56 mmol) in DCM (10 mL) using PPh₃ (1.026 g, 3.91 mmol), ethyl 2-(2-ethyl-6-hydroxyphenyl)acetate (180c) (0.889 g, 4.27 mmol), and a solution of DCAD (1.437 g, 3.91 mmol) in DCM to afford after workup and purification using method-AU, ethyl 2-(2-((5-bromo-1-cyclobutyl-1H-indazol-3-yl)methoxy)-6-ethylphenyl)acetate (180e) (560 mg, 33.4% yield) as a white solid; MS (ES+): 471.1 (M+1).

### Step-4: Preparation of ethyl 2-(2-((5-(1-aminoisoquinolin-7-yl)-1-cyclobutyl-1H-indazol-3-yl)methoxy)-6-ethylphenyl)acetate (180f)

Compound 180f was prepared according to the procedure reported in step-5 of scheme 1 from ethyl 2-(2-((5-bromo-1-cyclobutyl-1H-indazol-3-yl)methoxy)-6-ethylphenyl)acetate (180e) (275 mg, 0.583 mmol) in dioxane/MeTHF (10 mL, 1:1) using 1-aminoisoquinolin-7-ylboronic acid (87a) (143 mg, 0.758 mmol), K₃PO₄ (2M aqueous solution, 0.583 mL, 2.334 mmol), PCy₃ (32.7 mg, 0.117 mmol), Pd₂(dba)₃ (53.4 mg, 0.058 mmol) and PdCl₂(dppf)-CH₂Cl₂ adduct (47.6 mg, 0.058 mmol) to afford after workup and purification using method-AE, ethyl 2-(2-((5-(1-aminoisoquinolin-7-yl)-1-cyclobutyl-1H-indazol-3-yl)methoxy)-6-ethylphenyl)acetate (180f) (75.2 mg, 24.11% yield) as a yellow solid. MS (ES+): 535.2 (M+1).

### Step-5: Preparation of 2-(2-((5-(1-aminoisoquinolin-7-yl)-1-cyclobutyl-1H-indazol-3-yl)methoxy)-6-ethylphenyl)acetic acid (180g)

Compound 180g was prepared according to the procedure and method of purification reported in step-6 of scheme 7, from ethyl 2-(2-((5-(1-aminoisoquinolin-7-yl)-1-cyclobutyl-1H-indazol-3-yl)methoxy)-6-ethylphenyl)acetate (180f) (76 mg, 0.142 mmol) in THF (3 mL) using a solution of LiOH·H₂O (17.90 mg, 0.426 mmol) in water (1 mL) to afford after workup and purification using method-M, 2-(2-((5-(1-aminoisoquinolin-7-yl)-1-cyclobutyl-1H-indazol-3-yl)methoxy)-6-ethylphenyl)acetic acid (180g) (30.3 mg, 42.1% yield) HCl salt as a white solid; H NMR (300 MHz, DMSO-d₆) δ 13.28 (s, 1H, D₂O exchangeable), 12.11 (s, 1H, D₂O exchangeable), 9.15 (s, 2H, D₂O exchangeable), 8.94 (d, 1H), 8.39 (dd, J=8.5, 1.6 Hz, 1H), 8.28 (s, 1H), 8.08-7.95 (m, 2H), 7.89 (d, J=8.9 Hz, 1H), 7.69 (d, J=6.9 Hz, 1H), 7.31-7.16 (m, 2H), 7.20-7.11 (m, 1H), 6.84 (dd, J=7.4, 1.3 Hz, 1H), 5.47 (s, 2H), 5.45-5.28 (m, 1H), 3.59 (s, 2H), 2.78-2.62 (m, 2H), 2.58-2.51 (m, 2H), 2.54-2.46 (m, 2H), 1.99-1.81 (m, 2H), 1.09 (t, J=7.5 Hz, 3H); MS (ES+): 507.3 (M+1).

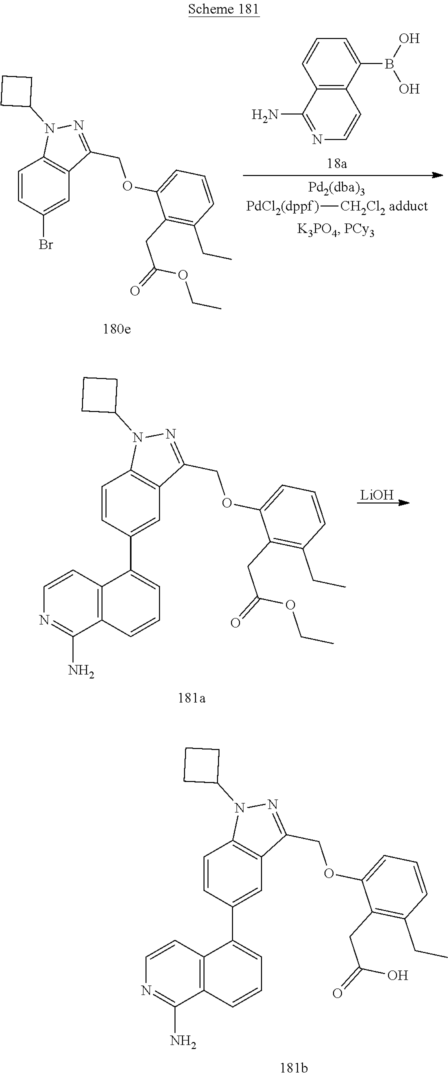

### Preparation of 2-(2-((5-(1-aminoisoquinolin-5-yl)-1-cyclobutyl-1H-indazol-3-yl)methoxy)-6-ethylphenyl)acetic acid (181b)

### Step 1: Preparation of ethyl 2-(2-((5-(1-aminoisoquinolin-5-yl)-1-cyclobutyl-1H-indazol-3-yl)methoxy)-6-ethylphenyl)acetate (181a)

Compound 181a was prepared according to the procedure reported in step-5 of scheme 1 from ethyl 2-(2-((5-bromo-1-cyclobutyl-1H-indazol-3-yl)methoxy)-6-ethylphenyl)acetate (180e) (275 mg, 0.583 mmol) in dioxane/MeTHF (25 mL, 9:1) using (1-aminoisoquinolin-5-yl)boronic acid (18a) (110 mg, 0.583 mmol), K₃PO₄ (2M aqueous solution, 0.583 mL, 2.334 mmol), PCy₃ (0.033 g, 0.117 mmol), Pd₂(dba)₃ (0.053 g, 0.058 mmol) and PdCl₂(dppf)-CH₂Cl₂ adduct (48 mg, 0.058 mmol) to afford after workup and purification using method-AE, ethyl 2-(2-((5-(1-aminoisoquinolin-5-yl)-1-cyclobutyl-1H-indazol-3-yl)methoxy)-6-ethylphenyl)acetate (181a) (46.3 mg, 14.84% yield) as a yellow solid; MS (ES+): 535.2 (M+1).

### Step 2: Preparation of 2-(2-((5-(1-aminoisoquinolin-5-yl)-1-cyclobutyl-1H-indazol-3-yl)methoxy)-6-ethylphenyl)acetic acid (181b)

Compound 181b was prepared according to the procedure reported step-2 of scheme 1 from ethyl 2-(2-((5-(1-aminoisoquinolin-5-yl)-1-cyclobutyl-1H-indazol-3-yl)methoxy)-6-ethylphenyl)acetate (181a) (46 mg, 0.086 mmol) in THF (3 mL) using a solution of LiOH·H₂O (10.83 mg, 0.258 mmol) in water (1 mL) to afford after workup and purification using method-M, 2-(2-((5-(1-aminoisoquinolin-5-yl)-1-cyclobutyl-1H-indazol-3-yl)methoxy)-6-ethylphenyl)acetic acid (181b) (11.2 mg, 25.7% yield) HCl salt as a white solid; ¹H NMR (300 MHz, DMSO-d₆) δ 13.04 (s, 1H, D₂O exchangeable), 11.99 (s, 1H, D₂O exchangeable), 9.03 (s, 2H, D₂O exchangeable), 8.58 (d, J=8.2 Hz, 1H), 7.97-7.79 (m, 4H), 7.61 (d, J=7.2 Hz, 1H), 7.46 (dd, J=8.6, 1.7 Hz, 1H), 7.24-7.14 (m, 1H), 7.14-7.07 (m, 1H), 6.98 (d, J=7.2 Hz, 1H), 6.81 (d, J=7.1 Hz, 1H), 5.43 (s, 2H), 5.41-5.28 (m, 1H), 3.54 (s, 2H), 2.72-2.59 (m, 2H), 2.54-2.52 (m, 2H), 2.54-2.45 (m, 2H), 1.99-1.82 (m, 2H), 1.06 (t, J=7.5 Hz, 3H); MS (ES+): 507.3 (M+1).

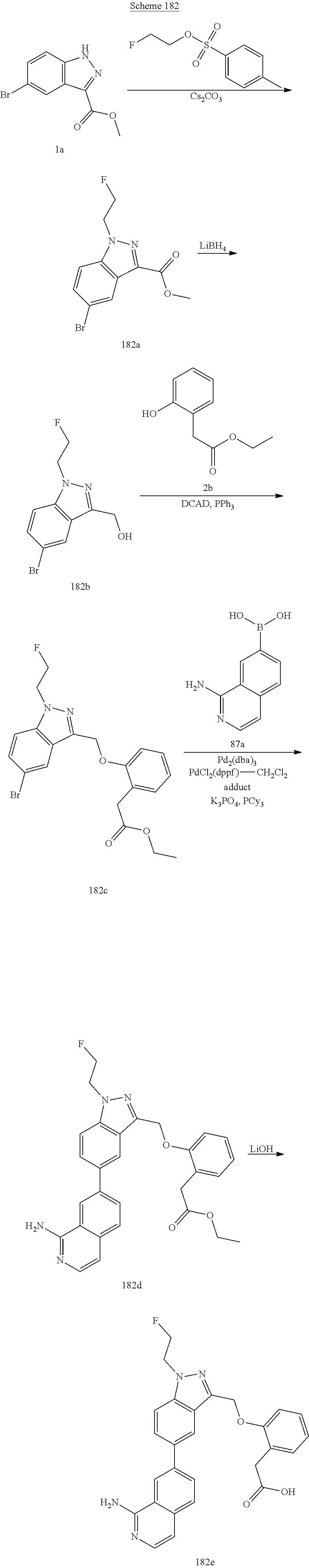

### Preparation of 2-(2-((5-(1-aminoisoquinolin-7-yl)-1-(2-fluoroethyl)-1H-indazol-3-yl)methoxy)phenyl)acetic acid (182e)

### Step-1: Preparation of methyl 5-bromo-1-(2-fluoroethyl)-1H-indazole-3-carboxylate (182a)

Compound 182a was prepared according to the procedure reported in step-2 of scheme 86 from methyl 5-bromo-1H-indazole-3-carboxylate (1a) (5.77 g, 22.60 mmol) in DMF (60 mL) using Cs₂CO₃ (14.73 g, 45.2 mmol) and 2-fluoroethyl 4-methylbenzenesulfonate (7.4 g, 33.9 mmol; CAS #383-50-6) to afford after workup and purification using method-A, methyl 5-bromo-1-(2-fluoroethyl)-1H-indazole-3-carboxylate (182a) (2.1 g, 6.97 mmol, 30.9% yield) as a yellow solid; MS (ES+): 301.00 (M+1).

### Step-2: Preparation of (5-bromo-1-(2-fluoroethyl)-1H-indazol-3-yl)methanol (182b)

Compound 182b was prepared according to the procedure reported in step-2 of scheme 119 from methyl 5-bromo-1-(2-fluoroethyl)-1H-indazole-3-carboxylate (182a) (2.08 g, 6.91 mmol) in THF (25 mL) using LiBH₄ (8.63 mL, 17.27 mmol) and MeOH (0.699 mL, 17.27 mmol) to afford after workup and purification using method-AW, (5-bromo-1-(2-fluoroethyl)-1H-indazol-3-yl)methanol (182b) (1.763 g, 93% yield) as a clear oil; MS (ES+): 273.0 (M+1).

### Step-3: Preparation of ethyl 2-(2-((5-bromo-1-(2-fluoroethyl)-1H-indazol-3-yl)methoxy)phenyl)acetate (182c)

Compound 182c was prepared according to the procedure reported in step-2 of scheme 2, from (5-bromo-1-(2-fluoroethyl)-1H-indazol-3-yl)methanol (182b) (1.700 g, 6.22 mmol) in DCM (25 mL) using PPh₃ (1.796 g, 6.85 mmol), ethyl 2-(2-hydroxyphenyl)acetate (2b) (1.346 g, 7.47 mmol) and a solution of DCAD (2.51 g, 6.85 mmol) in DCM to afford after workup and purification using method-AU, ethyl 2-(2-((5-bromo-1-(2-fluoroethyl)-1H-indazol-3-yl)methoxy)phenyl)acetate (182c) (1.315 g, 48.5% yield) as a white solid; MS (ES+): 435.0 (M+1).

### Step-4:Preparation of ethyl 2-(2-((5-(1-aminoisoquinolin-7-yl)-1-(2-fluoroethyl)-1H-indazol-3-yl)methoxy)phenyl)acetate (182d)

Compound 182d was prepared according to the procedure reported in step-5 of scheme 1 from ethyl 2-(2-((5-bromo-1-(2-fluoroethyl)-1H-indazol-3-yl)methoxy)phenyl)acetate (300 mg, 0.689 mmol) in dioxane/MeTHF (10 mL, 1:1) using 1-aminoisoquinolin-7-ylboronic acid (87a) (0.168 g, 0.896 mmol), K₃PO₄ (2M aqueous solution, 0.689 mL, 2.76 mmol), PCy₃ (0.039 g, 0.138 mmol), Pd₂(dba)₃ (63 mg, 0.069 mmol) and PdCl₂(dppf)-CH₂Cl₂ adduct (56 mg, 0.069 mmol) to afford after workup and purification using method-AE, ethyl 2-(2-((5-(1-aminoisoquinolin-7-yl)-1-(2-fluoroethyl)-1H-indazol-3-yl)methoxy)phenyl)acetate (182d) (145 mg, 42.2% yield) as a yellow solid; MS (ES+): 499.2 (M+1).

### Step-5: Preparation of 2-(2-((5-(1-aminoisoquinolin-7-yl)-1-(2-fluoroethyl)-1H-indazol-3-yl)methoxy)phenyl)acetic acid (182e)

Compound 182e was prepared according to the procedure reported step-2 of scheme 1 from ethyl 2-(2-((5-(1-aminoisoquinolin-7-yl)-1-(2-fluoroethyl)-1H-indazol-3-yl)methoxy)phenyl)acetate (182d) (145 mg, 0.291 mmol) in THF (3 mL) using a solution of LiOH·H₂O (36.6 mg, 0.873 mmol) in water (1 mL) to afford after workup and purification using method-M, 2-(2-((5-(1-aminoisoquinolin-7-yl)-1-(2-fluoroethyl)-1H-indazol-3-yl)methoxy)phenyl)acetic acid (182e) (40.1 mg, 29.3% yield) HCl salt as a white solid; ¹H NMR (300 MHz, DMSO-d₆) δ 13.16 (s, 1H, D₂O exchangeable), 12.23 (s, 1H, D₂O exchangeable), 9.13 (s, 2H, D₂O exchangeable), 8.96 (d, J=1.8 Hz, 1H), 8.42 (dd, J=8.5, 1.6 Hz, 1H, D₂O exchangeable), 8.33 (d, J=1.6 Hz, 1H), 8.09-7.97 (m, 2H), 7.91 (d, J=8.8 Hz, 1H), 7.70 (d, J=6.9 Hz, 1H), 7.36-7.16 (m, 4H), 7.00-6.89 (m, 1H), 5.50 (s, 2H), 5.03-4.83 (m, 2H), 4.79 (s, 2H), 3.54 (s, 2H); ¹⁹F NMR (282 MHz, DMSO-d₆) δ 16.30; MS (ES+): 471.2 (M+1); (ES−): 469.1 (M−1).

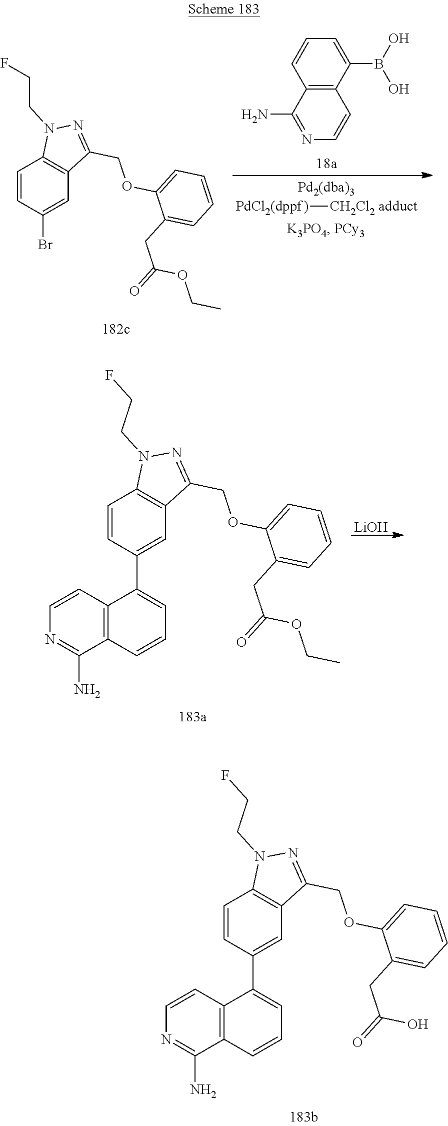

### Preparation of 2-(2-((5-(1-aminoisoquinolin-5-yl)-1-(2-fluoroethyl)-1H-indazol-3-yl)methoxy)phenyl)acetic acid (183b)

### Step-1: Preparation of ethyl 2-(2-((5-(1-aminoisoquinolin-5-yl)-1-(2-fluoroethyl)-1H-indazol-3-yl)methoxy)phenyl)acetate (183a)

Compound 183a was prepared according to the procedure reported in step-5 of scheme 1 from ethyl 2-(2-((5-bromo-1-(2-fluoroethyl)-1H-indazol-3-yl)methoxy)phenyl)acetate (182c) (300 mg, 0.689 mmol) in dioxane/MeTHF (25 mL, 9:1) using (I-aminoisoquinolin-5-yl)boronic acid (18a) (130 mg, 0.689 mmol), K₃PO₄ (2M aqueous solution, 0.689 mL, 2.76 mmol), PCy₃ (0.039 g, 0.138 mmol), Pd₂(dba)₃ (63 mg, 0.069 mmol) and PdCl₂(dppf)-CH₂Cl₂ adduct (56 mg, 0.069 mmol) to afford after workup and purification using method-AE, ethyl 2-(2-((5-(1-aminoisoquinolin-5-yl)-1-(2-fluoroethyl)-1H-indazol-3-yl)methoxy)phenyl)acetate (183a) (127 mg, 37.0% yield) as a yellow solid; MS (ES+): 499.2 (M+1).

### Step 2: Preparation of 2-(2-((5-(1-aminoisoquinolin-5-yl)-1-(2-fluoroethyl)-1H-indazol-3-yl)methoxy)phenyl)acetic acid (183b)

Compound 183b was prepared according to the procedure reported step-2 of scheme 1 from ethyl 2-(2-((5-(1-aminoisoquinolin-5-yl)-1-(2-fluoroethyl)-1H-indazol-3-yl)methoxy)phenyl)acetate (183a) (127 mg, 0.255 mmol) in THF (3 mL) using a solution of LiOH·H₂O (19 mg, 0.765 mmol) in water (1 mL) to afford after workup and purification using method-M, 2-(2-((5-(1-aminoisoquinolin-5-yl)-1-(2-fluoroethyl)-1H-indazol-3-yl)methoxy)phenyl)acetic acid (183b) (19.5 mg, 0.041 mmol) HCl salt as a white solid; 1H NMR (300 MHz, DMSO-d₆) δ 13.19 (s, 1H, D₂O exchangeable), 12.02 (s, 1H, D₂O exchangeable), 9.06 (s, 2H, D₂O exchangeable), 8.60 (d, J=8.3 Hz, 1H), 8.00-7.79 (m, 4H), 7.63 (d, J=7.2 Hz, 1H), 7.51 (dd, J=8.7, 1.7 Hz, 1H), 7.28-7.21 (m, 2H), 7.21-7.11 (m, 1H), 6.99 (d, J=7.2 Hz, 1H), 6.94-6.86 (m, 1H), 5.46 (s, 2H), 5.04-4.84 (m, 2H), 4.81 (s, 2H), 3.49 (s, 2H); ¹⁹F NMR (282 MHz, DMSO-d₆) δ 16.46; MS (ES+): 471.2 (M+1); (ES−): 469.1 (M−1).

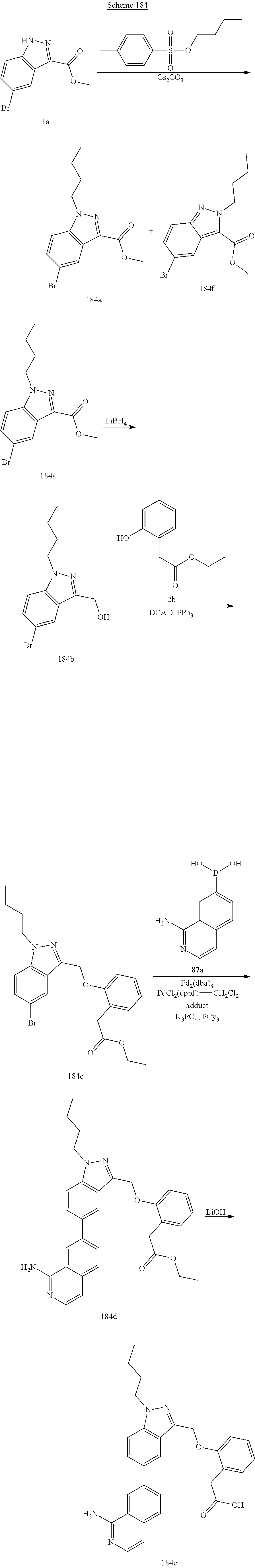

### Preparation of 2-(2-(5-(1-aminoisoquinolin-7-yl)-1-butyl-1H-indazol-3-yl)methoxy)phenyl)acetic acid (184e)

### Step-1: Preparation of methyl 5-bromo-1-butyl-1H-indazole-3-carboxylate (184a) and methyl 5-bromo-2-butyl-2H-indazole-3-carboxylate (184f)

Compounds 184a and 184f were prepared according to the procedure reported in step-2 of scheme 86 from methyl 5-bromo-1H-indazole-3-carboxylate (1a) (5.59 g, 21.90 mmol) in DMF (80 mL) using Cs₂CO₃ (14.27 g, 43.8 mmol) and butyl 4-methylbenzenesulfonate (10 g, 43.8 mmol) to afford after workup and purification using method-A, methyl 5-bromo-1-butyl-1H-indazole-3-carboxylate (184a) (3.4 g, 49.9% yield) as a yellow solid; 1H NMR (300 MHz, DMSO-d₆) δ 8.19 (dd, J=1.9, 0.7 Hz, 1H), 7.86 (dd, J=9.0, 0.7 Hz, 1H), 7.62 (dd, J=9.0, 1.9 Hz, 1H), 4.52 (t, J=7.0 Hz, 2H), 3.92 (s, 3H), 1.90-1.72 (m, 2H), 1.31-1.10 (m, 2H), 0.86 (t, J=7.3 Hz, 3H); MS (ES+): 311.00 & 313.00 (M+1); 333.00 & 335.00 (M+Na) and methyl 5-bromo-2-butyl-2H-indazole-3-carboxylate (1841) (2.1 g, 30.8% yield) as a yellow solid; ¹H NMR (300 MHz, DMSO-d₆) δ 8.15 (dd, J=1.9, 0.7 Hz, 1H), 7.79 (dd, J=9.1, 0.8 Hz, 1H), 7.49 (dd, J=9.1, 1.9 Hz, 1H), 4.82 (t, J=7.2 Hz, 2H), 3.98 (s, 3H), 1.97-1.76 (m, 2H), 1.27 (hept, J=7.3 Hz, 2H), 0.89 (t, J=7.4 Hz, 3H); MS (ES+): 311.00 & 313.00 (M+1).

### Step-2: Preparation of (5-bromo-1-butyl-1H-indazol-3-yl)methanol (184b)

Compound 184b was prepared according to the procedure reported in step-2 of scheme 119 from methyl 5-bromo-1-butyl-1H-indazole-3-carboxylate (184a) (3.4 g, 10.93 mmol) in THF (25 mL) using LiBH₄ (13.66 mL, 27.3 mmol) and MeOH (1.105 mL, 27.3 mmol) to afford after workup and purification using method-K. (5-bromo-1-butyl-1H-indazol-3-yl)methanol (184b) (1.175 g, 38.0% yield) as a clear oil; MS (ES+): 283.00/285.0 (M+1).

### Step-3: Preparation of ethyl 2-(2-((5-bromo-1-butyl-1H-indazol-3-yl)methoxy)phenyl)acetate (184c)

Compound 184c was prepared according to the procedure reported in step-2 of scheme 2 from (5-bromo-1-butyl-1H-indazol-3-yl)methanol (184b) (1.175 g, 4.15 mmol) in DCM (20 mL) using PPh₃ (1.197 g, 4.56 mmol), ethyl 2-(2-hydroxyphenyl)acetate (2b) (0.897 g, 4.98 mmol) and a solution of DCAD (1.676 g, 4.56 mmol) in DCM to afford after workup and purification using method-AV, ethyl 2-(2-((5-bromo-1-butyl-1H-indazol-3-yl)methoxy)phenyl)acetate (184c) (1.303 g, 70.5% yield) as a white solid; MS (ES+): 445.1/447.1 (M+1).

### Step-4: Preparation of ethyl 2-(2-((5-(1-aminoisoquinolin-7-yl)-1-butyl-1H-indazol-3-yl)methoxy)phenyl)acetate (184d)

Compound 184d was prepared according to the procedure reported in step-5 of scheme 1 from ethyl 2-(2-((5-bromo-1-butyl-1H-indazol-3-yl)methoxy)phenyl)acetate (184c) (650 mg, 1.460 mmol) in dioxane/MeTHF (10 ml, 1:1) using 1-aminoisoquinolin-7-ylboronic acid (87a) (357 mg, 1.897 mmol), K₃PO₄ (2M aqueous solution, 1.460 mL, 5.84 mmol), PCy₃ (82 mg, 0.292 mmol), Pd₂(dba)₃ (134 mg, 0.146 mmol) and PdCl₂(dppf)-CH₂Cl₂ adduct (119 mg, 0.146 mmol) to afford after workup and purification using method-AE, ethyl 2-(2-((5-(1-aminoisoquinolin-7-yl)-1-butyl-1H-indazol-3-yl)methoxy)phenyl)acetate (184d) (360 mg, 48.5% yield) as a yellow solid; MS (ES+): 509.3 (M+1).

### Step-5: Preparation of 2-(2-((5-(1-aminoisoquinolin-7-yl)-1-butyl-1H-indazol-3-yl)methoxy)phenyl)acetic acid (184e)

Compound 184e was prepared according to the procedure reported step-2 of scheme 1 from ethyl 2-(2-((5-(1-aminoisoquinolin-7-yl)-1-butyl-1H-indazol-3-yl)methoxy)phenyl)acetate (184d) (360 mg, 0.708 mmol) in THF (3 mL) using a solution of LiOH·H₂O (89 mg, 2.123 mmol) in water (1 mL) to afford after workup and purification using method-M, 2-(2-((5-(1-aminoisoquinolin-7-yl)-1-butyl-1H-indazol-3-yl)methoxy)phenyl)acetic acid (184e) (27 mg, 7.94% yield) HCl salt as a white solid; ¹H NMR (300 MHz, DMSO-d₆) δ 13.04 (s, 1H, DO exchangeable), 12.37 (s, 1H, D₂O exchangeable), 9.07 (s, 2H, D₂O) exchangeable), 8.94 (d, J=1.7 Hz, 1H), 8.40 (dd, J=8.5, 1.6 Hz, 1H), 8.30 (d, J=1.6 Hz, 1H), 8.06-7.96 (m, 2H), 7.88 (d, J=8.9 Hz, 1H), 7.69 (d, J=6.9 Hz, 1H), 7.30-7.23 (m, 3H), 7.23-7.17 (m, 1H), 6.95-6.88 (m, 1H), 5.48 (s, 2H), 4.47 (t, J=6.8 Hz, 2H), 3.52 (s, 2H), 1.92-1.76 (m, 2H), 1.34-1.18 (m, 2H), 0.89 (t, J=7.3 Hz, 3H); MS (ES+): 481.2 (M+1); (ES−): 479.1 (M−1).

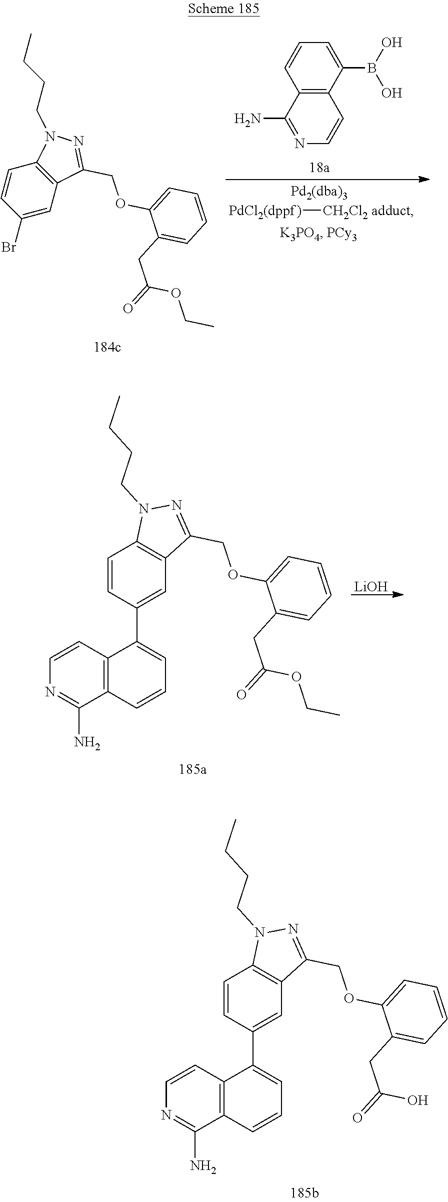

### Preparation of 2-(2-((5-(1-aminoisoquinolin-5-yl)-1-butyl-1H-indazol-3-yl)methoxy)phenyl)acetic acid (185b)

### Step-1: Preparation of ethyl 2-(2-((5-(1-aminoisoquinolin-5-yl)-1-butyl-1H-indazol-3-yl)methoxy)phenyl)acetate (185a)

Compound 185a was prepared according to the procedure reported in step-5 of scheme 1 from ethyl 2-(2-((5-bromo-1-butyl-1H-indazol-3-yl)methoxy)phenyl)acetate (184c) (650 mg, 1.460 mmol) in dioxane/MeTHF (25 mL, 9:1) using (1-aminoisoquinolin-5-yl)boronic acid (18a) (274 mg, 1.460 mmol), K₃PO₄ (2M aqueous solution, 1.460 mL, 5.84 mmol), PCy₃ (82 mg, 0.292 mmol), Pd₂(dba)₃ (134 mg, 0.146 mmol) and PdCl₂(dppf)-CH₂Cl₂ adduct (119 mg, 0.146 mmol) to afford after workup and purification using method-AE, ethyl 2-(2-((5-(1-aminoisoquinolin-5-yl)-1-butyl-1H-indazol-3-yl)methoxy)phenyl)acetate (185a) (630 mg, 85% yield) as a yellow solid; MS (ES+): 509.3 (M+1).

### Step-2: Preparation of 2-(2-((5-(1-aminoisoquinolin-5-yl)-1-butyl-1H-indazol-3-yl)methoxy)phenyl)acetic acid (185b)

Compound 185b was prepared according to the procedure reported step-2 of scheme 1 from ethyl 2-(2-((5-(1-aminoisoquinolin-5-yl)-1-butyl-1H-indazol-3-yl)methoxy)phenyl)acetate (185a) (630 mg, 1.24 mmol) in THF (4 mL) using a solution of LiOH·H₂O (156 mg, 3.72 mmol) in water (1 mL) to afford after workup and purification using method-M, 2-(2-((5-(1-aminoisoquinolin-5-yl)-1-butyl-1H-indazol-3-yl)methoxy)phenyl)acetic acid (185b) (61 mg, 10% yield) HCl salt as a white solid; ¹H NMR (300 MHz, DMSO-d₆) δ 13.24 (s, 1H, D₂O exchangeable), 11.99 (s, 1H, D₂O exchangeable), 9.07 (s, 2H, D₂O exchangeable), 8.60 (d, J=8.3 Hz, 1H), 7.95 (dd, J=7.4, 1.1 Hz, 1H), 7.90-7.81 (m, 3H), 7.62 (d, J=7.2 Hz, 1H), 7.47 (dd, J=8.7, 1.6 Hz, 1H), 7.26-7.21 (m, 2H), 7.20-7.14 (m, 1H), 6.98 (d, J=7.2 Hz, 1H), 6.94-6.85 (m, 1H), 5.45 (s, 2H), 4.48 (t, J=6.9 Hz, 2H), 3.47 (s, 2H), 1.94-1.78 (m, 2H), 1.38-1.19 (m, 2H), 0.91 (t, J==7.3 Hz, 3H); MS (ES+): 481.2 (M+1); (ES−): 479.1 (M−1); Analysis calculated for C₂₉H₂₈N₄O₃·H₂O·1.1 HCl: C, 64.66; H, 5.82; Cl, 7.24; N, 10.40; Found: C, 64.57; H, 5.71; Cl, 7.28; N, 10.33.

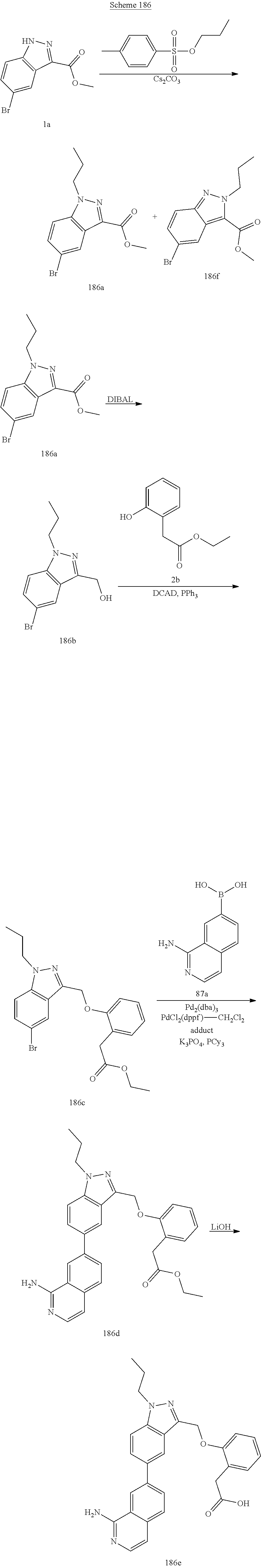

### Preparation of 2-(2-((5-(1-aminoisoquinolin-7-yl)-1-propyl-1H-indazol-3-yl)methoxy)phenyl)acetic acid (186e)

### Step-1: Preparation of methyl 5-bromo-1-propyl-1H-indazole-3-carboxylate (186a) and methyl 5-bromo-2-propyl-2H-indazole-3-carboxylate (186f)

Compounds 186a and 186f were prepared according to the procedure reported in step-2 of scheme 86 from methyl 5-bromo-1H-indazole-3-carboxylate (1a) (5.95 g, 23.33 mmol) in DMF (60 mL) using Cs₂CO₃ (15.21 g, 46.7 mmol) and propyl 4-methylbenzenesulfonate (5 g, 23.33 mmol; CAS #599-91-7) to afford after workup and purification using method-A, methyl 5-bromo-1-propyl-1H-indazole-3-carboxylate (186a) (4 g, 57.7% yield) as a white solid; 1H NMR (300 MHz, DMSO-d₆) δ 8.22-8.10 (m, 1H), 7.88-7.79 (m, 1H), 7.63-7.54 (m, 1H), 4.47 (t, J=7.0 Hz, 2H), 3.92 (s, 3H), 1.85 (h, J=7.2 Hz, 2H), 0.81 (t, J=7.4 Hz, 3H); MS (ES+): 297.00 & 299.00 (M+1); 319.00 & 321.00 (M+Na) and methyl 5-bromo-2-propyl-2H-indazole-3-carboxylate (186f) (2.7 g, 38.9% yield) as a white solid; 1H NMR (300 MHz, DMSO-d₆) δ 8.16 (dd, J=1.9, 0.8 Hz, 1H), 7.79 (dd, J=9.1, 0.8 Hz, 1H), 7.49 (dd, J=9.1, 1.9 Hz, 1H), 4.79 (t, J=7.2 Hz, 2H), 3.99 (s, 3H), 1.91 (h, J=7.3 Hz, 2H), 0.86 (t, J=7.4 Hz, 3H); MS (ES+): 297.00 & 299.00 (M+1).

### Step-2: Preparation of (5-bromo-1-propyl-1H-indazol-3-yl)methanol (186b)

Compound 186b was prepared according to the procedure reported in step-1 of scheme 2 from methyl 5-bromo-1-propyl-1H-indazole-3-carboxylate (186a) (3.85 g, 12.96 mmol) in DCM (75 mL) using DIBAL (1M solution in DCM, 32.4 mL, 32.4 mmol) to afford after workup and purification using method-F, (5-bromo-1-propyl-1H-indazol-3-yl)methanol (186b) (2.325 g, 66.7% yield) as a light yellow oil; 1H NMR (300 MHz, DMSO-d₆) δ 8.05 (dd, J=1.8, 0.7 Hz, 1H), 7.65 (dd, J=9.0, 0.7 Hz, 1H), 7.48 (dd, J=8.9, 1.9 Hz, 1H), 5.31 (d, J=6.0 Hz, 1H), 4.75 (d, J=4.7 Hz, 2H), 4.31 (t, J=6.9 Hz, 2H), 1.81 (h, J=7.2 Hz, 2H), 0.82 (t, J=7.4 Hz, 3H); MS (ES+): 269.0 (M+1).

### Step-3: Preparation of ethyl 2-(2-((5-bromo-1-propyl-1H-indazol-3-yl)methoxy)phenyl)acetate (186c)

Compound 186c was prepared according to the procedure reported in step-2 of scheme 2 from (5-bromo-1-propyl-1H-indazol-3-yl)methanol (186b) (2.325 g, 8.64 mmol) in DCM (50 mL) using PPh₃ (2.492 g, 9.50 mmol), ethyl 2-(2-hydroxyphenyl)acetate (2b) (1.868 g, 10.37 mmol) and a solution of DCAD (3.49 g, 9.50 mmol) in DCM to afford after workup and purification using method-AU, ethyl 2-(2-((5-bromo-1-propyl-1H-indazol-3-yl)methoxy)phenyl)acetate (186c) (2.651 g, 71.1% yield) as a white solid. MS (ES+): 431.1/433.0 (M+1).

### Step-4: Preparation of ethyl 2-(2-((5-(1-aminoisoquinolin-7-yl)-1-propyl-1H-indazol-3-yl)methoxy)phenyl)acetate (186d)

Compound 186d was prepared according to the procedure reported in step-5 of scheme 1 from ethyl 2-(2-((5-bromo-1-propyl-1H-indazol-3-yl)methoxy)phenyl)acetate (186c) (550 mg, 1.275 mmol) in dioxane/MeTHF (10 mL, 1:1) using 1-aminoisoquinolin-7-ylboronic acid (87a) (312 mg, 1.66 mmol), K₃PO₄ (2M aqueous solution, 1.275 mL, 5.10 mmol), PCy₃ (71.5 mg, 0.255 mmol), Pd₂(dba)₃ (117 mg, 0.128 mmol) and PdCl₂(dppf)-CH₂Cl₂ adduct (104 mg, 0.128 mmol) to afford after workup and purification using method-AE, ethyl 2-(2-((5-(1-aminoisoquinolin-7-yl)-1-propyl-1H-indazol-3-yl)methoxy)phenyl)acetate (186d) (203 mg, 32.2% yield) as a yellow solid; MS (ES+): 495.20 (M+1).

### Step-5: Preparation of 2-(2-((5-(1-aminoisoquinolin-7-yl)-1-propyl-1H-indazol-3-yl)methoxy)phenyl)acetic acid (186e)

Compound 186e was prepared according to the procedure reported step-2 of scheme 1 from ethyl 2-(2-((5-(1-aminoisoquinolin-7-yl)-1-propyl-1H-indazol-3-yl)methoxy)phenyl)acetate (186d) (203 mg, 0.410 mmol) in THF (3 mL) using a solution of LiOH·H₂O ($1.7 mg, 1.231 mmol) in water (1 mL) to afford after workup and purification using method-M, 2-(2-((5-(1-aminoisoquinolin-7-yl)-1-propyl-1H-indazol-3-yl)methoxy)phenyl)acetic acid (186e) (29.4 mg, 15.35% yield) HCl salt as a white solid; ¹H NMR (300 MHz, DMSO-d₆) δ 12.91 (s, 1H, D₂O exchangeable), 12.24 (s, 1H, D₂O exchangeable), 9.07 (s, 2H, D₂O exchangeable), 8.94 (d, J=1.7 Hz, 1H), 8.40 (dd, J=8.5, 1.6 Hz, 1H), 8.30 (d, J=1.6 Hz, 1H), 8.08-7.95 (m, 2H), 7.89 (d, J=8.9 Hz, 1H), 7.69 (d, J=6.9 Hz, 1H), 7.33-7.23 (m, 3H), 7.23-7.15 (m, 1H), 6.97-6.86 (m, 1H), 5.48 (s, 2H), 4.44 (t, J=6.8 Hz, 2H), 3.53 (s, 2H), 1.98-1.79 (m, 2H), 0.85 (t, J=7.4 Hz, 3H); MS (ES+): 467.2 (M+1); (ES−): 465.2 (M−1).

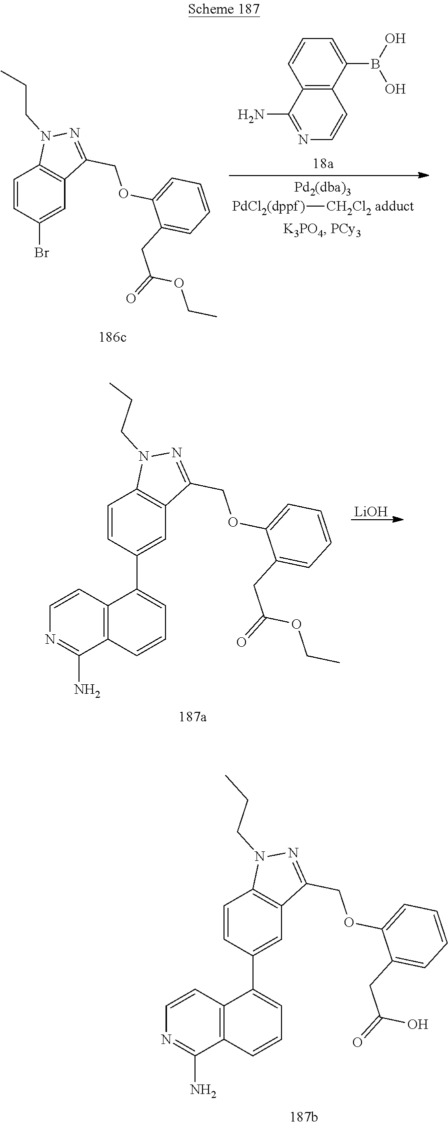

### Preparation of 2-(2-((5-(1-aminoisoquinolin-5-yl)-1-propyl-1H-indazol-3-yl)methoxy)phenyl)acetic acid (187b)

### Step-1: Preparation of ethyl 2-(2-((5-(1-aminoisoquinolin-5-yl)-1-propyl-1H-indazol-3-yl)methoxy)phenyl)acetate (187a)

Compound 187a was prepared according to the procedure reported in step-5 of scheme 1 from ethyl 2-(2-((5-bromo-1-propyl-1H-indazol-3-yl)methoxy)phenyl)acetate (186c) (550 mg, 0.128 mmol) in dioxane/MeTHF (25 mL, 9:1) using (1-aminoisoquinolin-5-yl)boronic acid (18a) (240 mg, 0.128 mmol), K₃PO₄ (2M aqueous solution, 1.275 mL, 5.101 mmol), PCy₃ (71.5 mg, 0.256 mmol), Pd₂(dba)₃ (117 mg, 0.13 mmol) and PdCl₂(dppf)-CH₂Cl₂ adduct (104 mg, 0.13 mmol) to afford after workup and purification using method-AE, ethyl 2-(2-((5-(1-aminoisoquinolin-5-yl)-1-propyl-1H-indazol-3-yl)methoxy)phenyl)acetate (187a) (259 mg, 0.041% yield) as a yellow solid; MS (ES+): 495.20 (M+1); (ES−): 493.10 (M−1).

### Step-2: Preparation of 2-(2-((5-(1-aminoisoquinolin-5-yl)-1-propyl-1H-indazol-3-yl)methoxy)phenyl)acetic acid (187b)

Compound 187b was prepared according to the procedure reported step-2 of scheme 1 from ethyl 2-(2-((5-(1-aminoisoquinolin-5-yl)-1-propyl-1H-indazol-3-yl)methoxy)phenyl)acetate (187a) (259 mg, 0.524 mmol) in THF (3 mL) using a solution of LiOH·H₂O (65.9 mg, 1.571 mmol) in water (1 mL) to afford after workup and purification using method-M, 2-(2-((5-(1-aminoisoquinolin-5-yl)-1-propyl-1H-indazol-3-yl)methoxy)phenyl)acetic acid (187b) (78.4 mg, 32.1% yield) HCl salt as a white solid; ¹H NMR (300 MHz, DMSO-d₆) δ 13.25 (s, 1H, D₂O exchangeable), 12.00 (s, 1H, D₂O exchangeable), 9.11 (s, 2H, D₂O exchangeable), 8.61 (d, J=8.3 Hz, 1H), 7.96 (dd, J=7.4, 1.1 Hz, 1H), 7.90-7.81 (m, 3H), 7.62 (d, J=7.2 Hz, 1H), 7.47 (dd, J=8.6, 1.7 Hz, 1H), 7.27-7.21 (m, 2H), 7.21-7.14 (m, 1H), 6.98 (d, J=7.3 Hz, 1H), 6.94-6.86 (m, 1H), 5.45 (s, 2H), 4.45 (t, J=6.9 Hz, 2H), 3.47 (s, 2H), 2.00-1.81 (m, 2H), 0.88 (t, J=7.4 Hz, 3H); MS (ES+): 467.2 (M+1); (ES−): 465.1 (M−1).

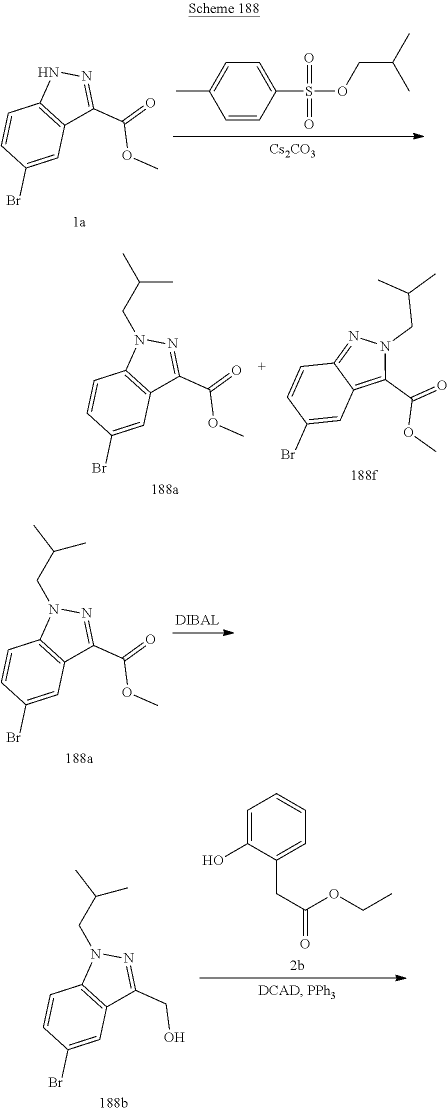

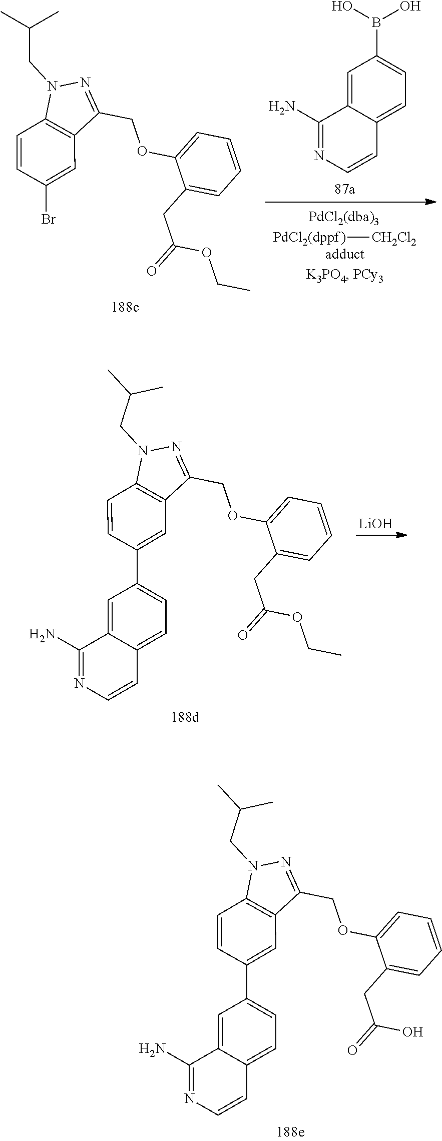

### Preparation of 2-(2-((5-(1-aminoisoquinolin-7-yl)-1-isobutyl-1H-indazol-3-yl)methoxy)phenyl)acetic acid (188e)

### Step-1: Preparation of methyl 5-bromo-1-isobutyl-1H-indazole-3-carboxylate (188a) and methyl 5-bromo-2-isobutyl-2H-indazole-3-carboxylate (188f)

Compounds 188a and 188f were prepared according to the procedure reported in step-2 of scheme 86 from methyl 5-bromo-1H-indazole-3-carboxylate (1a) (5.59 g, 21.90 mmol) in DMF (60 mL) using Cs₂CO₃ (14.27 g, 43.8 mmol) and isobutyl 4-methylbenzenesulfonate (5 g, 21.90 mmol) to afford after workup and purification using method-A, methyl 5-bromo-1-isobutyl-1H-indazole-3-carboxylate (188a) (4.2 g, 61.6% yield) as a white solid; ¹H NMR (300 MHz, DMSO-d₆) δ 8.20 (dd, J=1.9, 0.7 Hz, 1H), 7.89 (dd, J=9.0, 0.7 Hz, 1H), 7.62 (dd, J=9.0, 1.9 Hz, 1H), 4.35 (d, J=7.3 Hz, 2H), 3.93 (s, 3H), 2.23 (hept, J=6.8 Hz, 1H), 0.84 (d, J=6.7 Hz, 6H); MS (ES+): 311.00 & 313.00 (M+1); 333.00 & 335.00 (M+Na) and methyl 5-bromo-2-isobutyl-2H-indazole-3-carboxylate (188f) (2.4 g, 7.71 mmol, 35.2% yield) as a white solid; 1H NMR (300 MHz, DMSO-d₆) δ 8.20-8.09 (m, 1H), 7.83-7.73 (m, 1H), 7.53-7.42 (m, 1H), 4.66 (d, J=7.3 Hz, 2H), 3.98 (s, 3H), 2.26 (hept, J=6.8 Hz, 1H), 0.86 (d, J=6.7 Hz, 6H); MS (ES+): 311.00 & 313.05 (M+1).

### Step-2: Preparation of (5-bromo-1-isobutyl-1H-indazol-3-yl)methanol (188b)

Compound 188b was prepared according to the procedure reported in step-1 of scheme 2 from methyl 5-bromo-1-isobutyl-1H-indazole-3-carboxylate (188a) (4.2 g, 13.50 mmol) in DCM (75 mL) using DIBAL (1M solution in DCM, 33.7 mL, 33.7 mmol) to afford after workup and purification using method-F, (5-bromo-1-isobutyl-1H-indazol-3-yl)methanol (188b) (2.555 g, 66.9% yield) as a light yellow oil; 1H NMR (300 MHz, DMSO-d₆) δ 8.05 (dd, J=1.9, 0.7 Hz, 1H), 7.65 (dd, J=8.9, 0.7 Hz, 1H), 7.48 (dd, J=8.9, 1.9 Hz, 1H), 5.32 (t, J=5.9 Hz, 1H), 4.75 (d, J=5.9 Hz, 2H), 4.16 (d, J=7.2 Hz, 2H), 2.18 (dt, J=13.6, 6.8 Hz, 1H), 0.84 (d, J=6.7 Hz, 6H); MS (ES+): 283.0/285.0 (M+1).

### Step-3: Preparation of ethyl 2-(2-((5-bromo-1-isobutyl-1H-indazol-3-yl)methoxy)phenyl)acetate (188c)

Compound 188c was prepared according to the procedure reported in step-2 of scheme 2 from (5-bromo-1-isobutyl-1H-indazol-3-yl)methanol (188b) (2.555 g, 9.02 mmol) in DCM (50 mL) using PPh₃ (2.60 g, 9.93 mmol) and ethyl 2-(2-hydroxyphenyl)acetate (2b) (1.951 g, 10.83 mmol) and a solution of DCAD (3.64 g, 9.93 mmol) in DCM to afford after workup and purification using method-AU, ethyl 2-(2-((5-bromo-1-isobutyl-1H-indazol-3-yl)methoxy)phenyl)acetate (188c) (2.530 g, 63.0% yield) as a white solid; MS (ES+): 445.1/447.1.

### Step-4: Preparation of ethyl 2-(2-((5-(1-aminoisoquinolin-7-yl)-1-isobutyl-1H-indazol-3-yl)methoxy)phenyl)acetate (188d)

Compound 188d was prepared according to the procedure reported in step-5 of scheme 1 from ethyl 2-(2-((5-bromo-1-isobutyl-1H-indazol-3-yl)methoxy)phenyl)acetate (188c) (550 mg, 1.235 mmol) in dioxane/MeTHF (25 mL, 1:1) using 1-aminoisoquinolin-7-ylboronic acid (87a) (302 mg, 1.605 mmol), K₃PO₄ (2M aqueous solution, 1.235 mL, 4.94 mmol), PCy₃ (69.3 mg, 0.247 mmol), Pd₂(dba)₃ (113 mg, 0.123 mmol) and PdCl₂(dppf)-CH₂Cl₂ adduct (101 mg, 0.123 mmol) to afford after workup and purification using method-AE, ethyl 2-(2-((5-(1-aminoisoquinolin-7-yl)-1-isobutyl-1H-indazol-3-yl)methoxy)phenyl)acetate (188d) (300 mg, 47.8% yield) as a yellow solid; MS (ES+): 509.2 (M+1).

### Step-5: Preparation of 2-(2-((5-(1-aminoisoquinolin-7-yl)-1-isobutyl-1H-indazol-3-yl)methoxy)phenyl)acetic acid (188e)

Compound 188e was prepared according to the procedure reported step-2 of scheme 1 from ethyl 2-(2-((5-(1-aminoisoquinolin-7-yl)-1-isobutyl-1H-indazol-3-yl)methoxy)phenyl)acetate (188d) (300 mg, 0.590 mmol) in THF (3 mL) using a solution of LiOH·H₂O (74.3 mg, 1.770 mmol) in water (I mL) to afford after workup and purification using method-M, 2-(2-((5-(1-aminoisoquinolin-7-yl)-1-isobutyl-1H-indazol-3-yl)methoxy)phenyl)acetic acid (188e) (83.7 mg, 29.5% yield) HCl salt as a white solid; 1H NMR (300 MHz, DMSO-d₆) δ 13.14 (s, 1H, D₂O exchangeable), 12.50 (s, 1H, D₂O exchangeable), 9.13 (s, 2H, D₂O exchangeable), 8.99-8.90 (m, 1H), 8.40 (dd, J=8.5, 1.7 Hz, 1H), 8.34-8.27 (m, 1H), 8.08-7.94 (m, 2H), 7.89 (d, J=8.9 Hz, 1H), 7.69 (d, J=6.9 Hz, 1H), 7.32-7.23 (m, 3H), 7.23-7.16 (m, 1H), 6.99-6.82 (m, 1H), 5.49 (s, 2H), 4.29 (d, J=7.2 Hz, 2H), 3.52 (s, 2H), 2.35-2.16 (m, 1H), 0.88 (d, J=6.7 Hz, 6H); MS (ES+): 481.2 (M+1); (ES−): 479.2 (M−1).

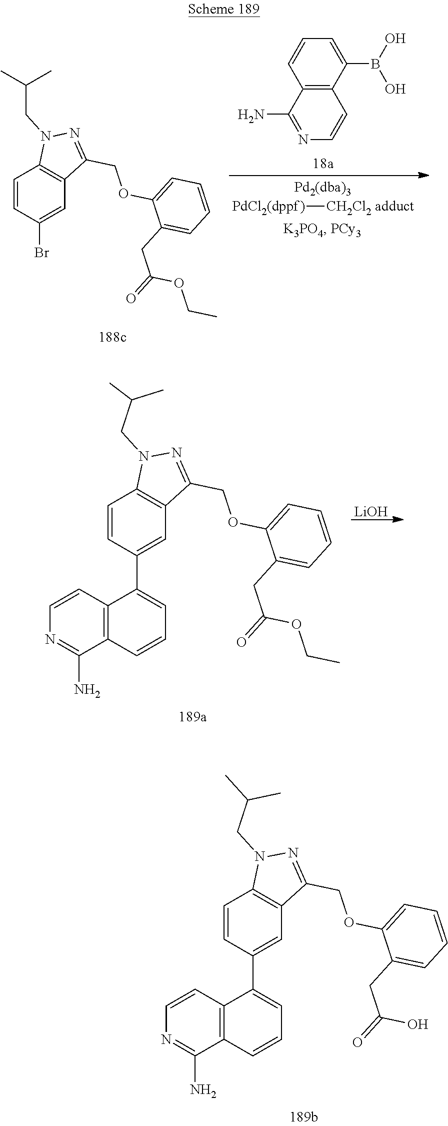

### Preparation of 2-(2-((5-(1-aminoisoquinolin-5-yl)-1-isobutyl-1H-indazol-3-yl)methoxy)phenyl)acetic acid (189b)

### Step-1:Preparation of ethyl 2-(2-((5-(1-aminoisoquinolin-5-yl)-1-isobutyl-1H-indazol-3-yl)methoxy)phenyl)acetate (189a)

Compound 189a was prepared according to the procedure reported in step-5 of scheme 1 from ethyl 2-(2-((5-bromo-1-isobutyl-1H-indazol-3-yl)methoxy)phenyl)acetate (188c) (550 mg, 1.235 mmol) in dioxane/MeTHF (25 mL, 9:1) using (1-aminoisoquinolin-5-yl)boronic acid (18a) (232 mg, 1.235 mmol), K₃PO₄ (2M aqueous solution, 1.235 mL, 4.94 mmol), PCy₃ (69.3 mg, 0.247 mmol), Pd₂(dba)₃ (113 mg, 0.123 mmol) and PdCl₂(dppf)-CH₂Cl₂ adduct (101 mg, 0.123 mmol) to afford after workup and purification using method-AE, ethyl 2-(2-((5-(1-aminoisoquinolin-5-yl)-1-isobutyl-1H-indazol-3-yl)methoxy)phenyl)acetate (189a) (193 mg, 30.7% yield) as a yellow solid; MS (ES+): 509.3 (M+1).

### Step 2: Preparation of 2-(2-((5-(1-aminoisoquinolin-5-yl)-1-isobutyl-1H-indazol-3-yl)methoxy)phenyl)acetic acid (189b)

Compound 189b was prepared according to the procedure reported step-2 of scheme 1 from ethyl 2-(2-((5-(1-aminoisoquinolin-5-yl)-1-isobutyl-1H-indazol-3-yl)methoxy)phenyl)acetate (189a) (193 mg, 0.379 mmol) in THF (3 mL) using a solution of LiOH·H₂O (47.8 mg, 1.138 mmol) in water (1 mL) to afford after workup and purification using method-M, 2-(2-((5-(1-aminoisoquinolin-5-yl)-1-isobutyl-1H-indazol-3-yl)methoxy)phenyl)acetic acid (189b) (59 mg, 32.4% yield) HCl salt as a white solid; 1H NMR (300 MHz, DMSO-d₆) δ 13.34 (s, 1H, D₂O exchangeable), 12.00 (s, 1H, D₂O exchangeable), 9.13 (s, 2H, D₂O exchangeable), 8.62 (d, J=8.3 Hz, 1H), 7.96 (dd, J=7.3, 1.1 Hz, 1H), 7.91-7.80 (m, 3H), 7.63 (d, J=7.2 Hz, 1H), 7.47 (dd, J=8.6, 1.7 Hz, 1H), 7.28-7.20 (m, 2H), 7.17 (dd, J=7.3, 1.4 Hz, 1H), 6.98 (d, J=7.2 Hz, 1H), 6.93-6.86 (m, 1H), 5.46 (s, 2H), 4.29 (d, J=7.1 Hz, 2H), 3.47 (s, 2H), 2.37-2.17 (m, 1H), 0.91 (d, J=6.7 Hz, 6H); MS (ES+): 481.2 (M+1); (ES−): 479.2 (M−1).

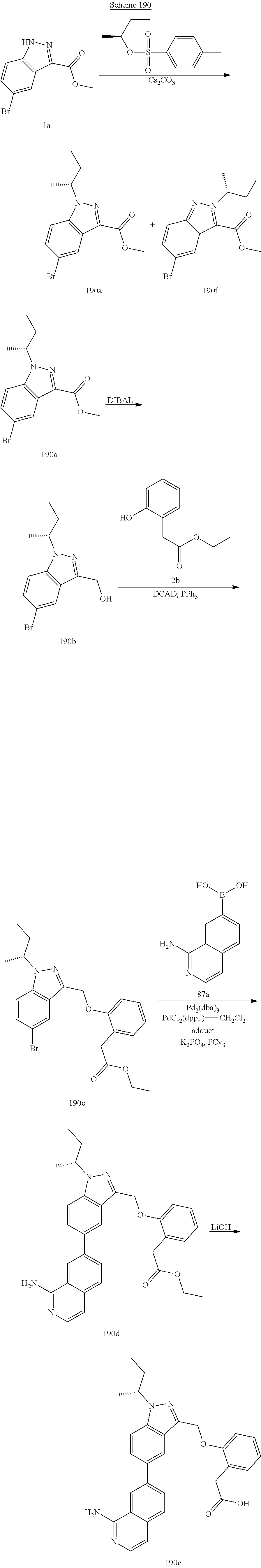

### Preparation of (R)-2-(2-((5-(1-aminoisoquinolin-7-yl)-1-(sec-butyl)-1H-indazol-3-yl)methoxy)phenyl)acetic acid (190e)

### Step-1: Preparation of (R)-methyl 5-bromo-1-(sec-butyl)-1H-indazole-3-carboxylate (190a) and (R)-methyl 5-bromo-2-(sec-butyl)-2H-indazole-3-carboxylate (190f)

Compounds 190a and 190f were prepared according to the procedure reported in step-2 of scheme 86 from methyl 5-bromo-1H-indazole-3-carboxylate (1a) (6.70 g, 26.3 mmol) in DMF (60 mL) using Cs₂CO₃ (17.13 g, 52.6 mmol) and (S)-(sec-butyl) 4-methylbenzenesulfonate (6 g, 26.3 mmol) to afford after workup and purification using method-A, (R)-methyl 5-bromo-1-(sec-butyl)-1H-indazole-3-carboxylate (190a) (4.05 g, 49.5% yield) as a white solid; 1H NMR (300 MHz, DMSO-d₆) δ 8.22 (dd, J=1.9, 0.7 Hz, 1H), 7.91 (dd, J=9.1, 0.7 Hz, 1H), 7.62 (dd, J=9.0, 1.9 Hz, 1H), 5.03-4.79 (m, 1H), 3.93 (s, 3H), 2.03-1.77 (m, 2H), 1.50 (d, J=6.6 Hz, 3H), 0.67 (t, J=7.4 Hz, 3H); MS (ES+): 311.00 & 313.00 (M+1); 333.00 & 335.00 (M+Na) and (R)-methyl 5-bromo-2-(sec-butyl)-2H-indazole-3-carboxylate (190f) (3.0 g, 36.7% yield) as a white solid; ¹H NMR (300 MHZ, DMSO-d₆) δ 8.16 (dd, J=1.9, 0.7 Hz, 1H), 7.81 (dd, J=9.0, 0.8 Hz, 1H), 7.48 (dd, J=9.1, 1.9 Hz, 1H), 5.79-5.59 (m, 1H), 3.99 (s, 3H), 2.13-1.81 (m, 2H), 1.53 (d, J=6.6 Hz, 3H), 0.70 (t, J=7.4 Hz, 3H); MS (ES+): 311.00 & 313.05 (M+1).

### Step-2: Preparation of (R)-(5-bromo-1-(sec-butyl)-1H-indazol-3-yl)methanol (190b)

Compound 190b was prepared according to the procedure reported in step-1 of scheme 2 from (R)-methyl 5-bromo-1-(sec-butyl)-1H-indazole-3-carboxylate (190a) (4.0 g, 12.85 mmol) in DCM (75 mL) using DIBAL (1M solution in DCM, 32.1 mL, 32.1 mmol) to afford after workup and purification using method-R, (R)-(5-bromo-1-(sec-butyl)-1H-indazol-3-yl)methanol (190b) (2.125 g, 58.4% yield) as a light yellow oil; MS (ES+): 283.00 (M+1).

### Step-3: Preparation of (R)-ethyl 2-(2-((5-bromo-1-(sec-butyl)-1H-indazol-3-yl)methoxy)phenyl)acetate (190c)

Compound 190c was prepared according to the procedure reported in step-2 of scheme 2 from (R)-(5-bromo-1-(sec-butyl)-1H-indazol-3-yl)methanol (190b) (2.0 g, 7.06 mmol) in DCM (50 mL) using PPh₃ (2.038 g, 7.77 mmol), ethyl 2-(2-hydroxyphenyl)acetate (2b) (1.527 g, 8.48 mmol) and a solution of DCAD (2.85 g, 7.77 mmol) in DCM to afford after workup and purification using method-AU. (R)-ethyl 2-(2-((5-bromo-1-(sec-butyl)-1H-indazol-3-yl)methoxy)phenyl)acetate (190c) (1.258 g, 40.0% yield) as a white solid. MS (ES+): 445.1/447.1 (M+1); (ES−): 443.0/445.0 (M−1).

### Step-4: Preparation of (R)-ethyl 2-(2-((5-(1-aminoisoquinolin-7-yl)-1-(sec-butyl)-1H-indazol-3-yl)methoxy)phenyl)acetate (190d)

Compound 190d was prepared according to the procedure reported in step-5 of scheme 1 from (R)-ethyl 2-(2-((5-bromo-1-(sec-butyl)-1H-indazol-3-yl)methoxy)phenyl)acetate (190c) (600 mg, 1.347 mmol) in dioxane/MeTHF (20 mL, 1:1) using 1-aminoisoquinolin-7-ylboronic acid (87a) (329 mg, 1.751 mmol), K₃PO₄ (2M aqueous solution, 1.347 mL, 5.39 mmol). PCy₃ (76 mg, 0.269 mmol), Pd₂(dba)₃ (123 mg, 0.135 mmol) and PdCl₂(dppf)-CH₂Cl₂ adduct (110 mg, 0.135 mmol) to afford after workup and purification using method-AE, (R)-ethyl 2-(2-((5-(1-aminoisoquinolin-7-yl)-1-(sec-butyl)-1H-indazol-3-yl)methoxy)phenyl)acetate (190d) (270 mg, 39.4% yield) as a yellow solid. MS (ES+): 509.30 (M+1).

### Step-5: Preparation of (R)-2-(2-((5-(1-aminoisoquinolin-7-yl)-1-(sec-butyl)-1H-indazol-3-yl)methoxy)phenyl)acetic acid (190e)

Compound 190e was prepared according to the procedure reported step-2 of scheme 1 from (R)-ethyl 2-(2-((5-(1-aminoisoquinolin-7-yl)-1-(sec-butyl)-1H-indazol-3-yl)methoxy)phenyl)acetate (190d) (270 mg, 0.531 mmol) in THF (3 mL) using a solution of LiOH·H₂O (66.8 mg, 1.593 mmol) in water (1 mL) to afford after workup and purification using method-M, (R)-2-(2-((5-(1-aminoisoquinolin-7-yl)-1-(sec-butyl)-1H-indazol-3-yl)methoxy)phenyl)acetic acid (190e) (11.4 mg, 4.47% yield) HCl salt as a white solid; ¹H NMR (300 MHz, DMSO-d₆) δ 13.36 (s, 1H, D₂O exchangeable), 12.14 (s, 1H, D₂O exchangeable), 9.19 (s, 2H, D₂O exchangeable), 8.96 (s, 1H), 8.41 (dd, J=8.5, 1.6 Hz, 1H), 8.30 (d, J=1.6 Hz, 1H), 8.07-7.98 (m, 1H), 7.98-7.86 (m, 2H), 7.69 (d, J=6.9 Hz, 1H), 7.36-7.24 (m, 3H), 7.24-7.16 (m, 1H), 6.91 (td, J=7.0, 1.7 Hz, 1H), 5.49 (s, 2H), 4.90-4.77 (m, 1H), 3.53 (s, 2H), 2.08-1.81 (m, 2H), 1.52 (d, J=6.5 Hz, 3H), 0.69 (t, J=7.3 Hz, 3H); MS (ES+): 481.2 (M+1); (ES−): 479.1 (M−1); Optical rotation: −23.158 [0.095, MeOH].

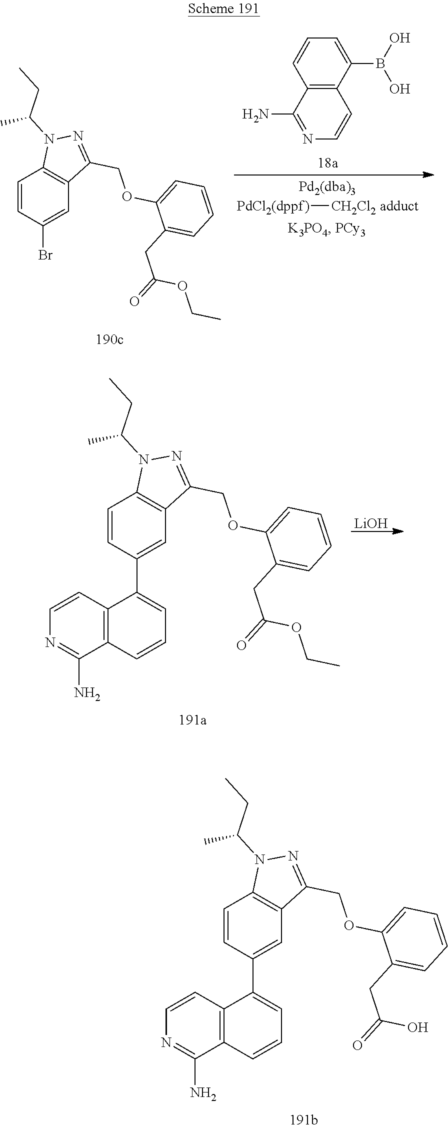

### Preparation of (R)-2-(2-((5-(1-aminoisoquinolin-5-yl)-1-(sec-butyl)˜1H-indazol-3-yl)methoxy)phenyl)acetic acid (191b)

### Step-1: Preparation of (R)-ethyl 2-(2-((5-(1-aminoisoquinolin-5-yl)-1-(sec-butyl)-1H-indazol-3-yl)methoxy)phenyl)acetate (191a)

Compound 191a was prepared according to the procedure reported in step-5 of scheme 1 from (R)-ethyl 2-(2-((5-bromo-1-(sec-butyl)-1H-indazol-3-yl)methoxy)phenyl)acetate (190c) (600 mg, 1.347 mmol) in dioxane/MeTHF (25 mL, 9:1) using (1-aminoisoquinolin-S-yl)boronic acid (18a) (253 mg, 1.347 mmol), K₃PO₄ (2M aqueous solution, 1.347 mL, 5.39 mmol), PCy₃ (76 mg, 0.269 mmol), Pd₂(dba)₃ (123 mg, 0.135 mmol) and PdCl₂(dppf)-CH₂Cl₂ adduct (110 mg, 0.135 mmol) to afford after workup and purification using method-AE, (R)-ethyl 2-(2-((5-(1-aminoisoquinolin-5-yl)-1-(sec-butyl)-1H-indazol-3-yl)methoxy phenyl)acetate (191a) (251 mg, 36.6% yield) as a yellow solid; MS (ES+): 509.20 (M+1).

### Step-2: Preparation of (R)-2-(2-((5-(1-aminoisoquinolin-5-yl)-1-(sec-butyl)-1H-indazol-3-yl)methoxy)phenyl)acetic acid (191b)

Compound 191b was prepared according to the procedure reported step-2 of scheme 1 from (R)-ethyl 2-(2-((5-(1-aminoisoquinolin-5-yl)-1-(sec-butyl)-1H-indazol-3-yl)methoxy)phenyl)acetate (191a) (251 mg, 0.494 mmol) in THF (3 mL) using a solution of LiOH·H₂O (62.1 mg, 1.481 mmol) in water (1 mL) to afford after workup and purification using method-M, (R)-2-(2-((5-(1-aminoisoquinolin-5-yl)-1-(sec-butyl)-1H-indazol-3-yl)methoxy)phenyl)acetic acid (191b) (56.8 mg, 23.95% yield) HCl salt as a white solid; ¹H NMR (300 MHz, DMSO-d₆) δ 13.50 (s, 1H, D₂O exchangeable), 12.05 (s, 1H, D₂O exchangeable), 9.26 (s, 2H, D₂O exchangeable), 8.65 (d, J=8.3 Hz, 1H), 7.96 (dd, J=7.4, 1.1 Hz, 1H), 7.92-7.81 (m, 3H), 7.63 (d, J=7.3 Hz, 1H), 7.45 (dd, J=8.6, 1.7 Hz, 1H), 7.28-7.20 (m, 2H), 7.20-7.14 (m, 1H), 6.99 (d, J=7.2 Hz, 1H), 6.92-6.85 (m, 1H), 5.46 (s, 2H), 4.90-4.75 (m, 1H), 3.48 (s, 2H), 2.12-1.96 (m, 1H), 1.96-1.80 (m, 1H), 1.53 (d, J=6.5 Hz, 3H), 0.72 (t, J=7.3 Hz, 3H); MS (ES+): 481.2 (M+1); (ES−): 479.1 (M−1); Optical rotation: −11.667 [0.12, MeOH].

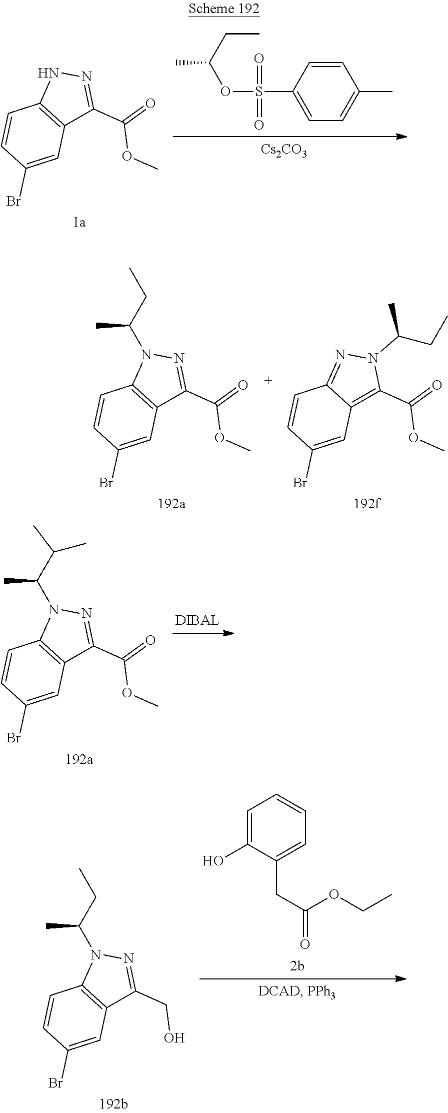

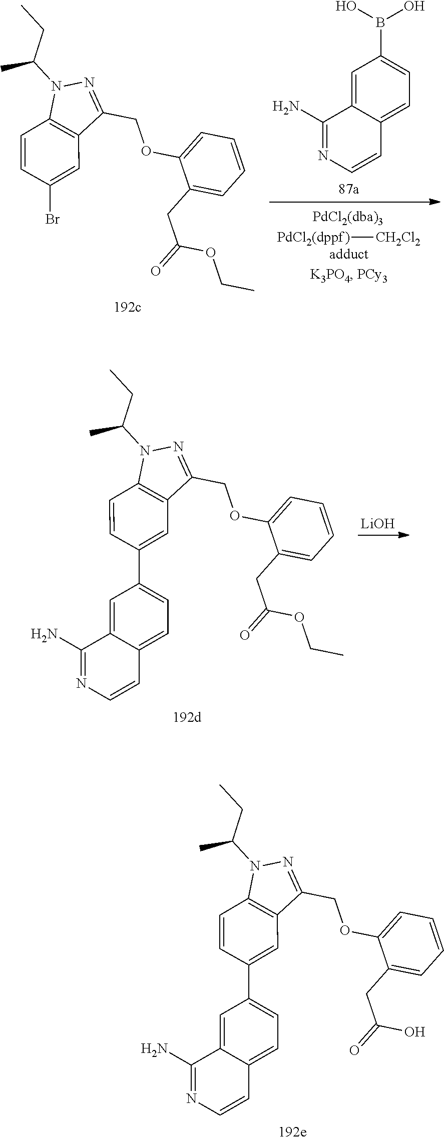

### Preparation of (S)-2-(2-((5-(1-aminoisoquinolin-7-yl)-1-(sec-butyl)-1H-indazol-3-yl)methoxy)phenyl)acetic acid (192e)

### Step-1: Preparation of (S)-methyl 5-bromo-1-(sec-butyl)-1H-indazole-3-carboxylate (192a) and (S)-methyl 5-bromo-2-(sec-butyl)-2H-indazole-3-carboxylate (192f)

Compounds 192a and 192f were prepared according to the procedure reported in step-2 of scheme 86 from methyl 5-bromo-1H-indazole-3-carboxylate (1a) (6.70 g, 26.3 mmol) in DMF (60 mL) using Cs₂CO₃ (17.13 g, 52.6 mmol) and (S)-(sec-butyl) 4-methylbenzenesulfonate (6 g, 26.3 mmol) to afford after workup and purification using method-A, (S)-methyl 5-bromo-1-(sec-butyl)-1H-indazole-3-carboxylate (192a) (4.03 g, 49.3% yield) as a white solid; 1H NMR (300 MHz, DMSO-d₆) δ 8.22 (dd, J=1.9, 0.7 Hz, 1H), 7.91 (dd, J=9.1, 0.7 Hz, 1H), 7.62 (dd, J=9.0, 1.9 Hz, 1H), 4.97-4.82 (m, 1H), 3.93 (s, 3H), 2.05-1.78 (m, 2H), 1.50 (d, J=6.7 Hz, 3H), 0.67 (t, J=7.4 Hz, 3H); MS (ES+): 311.05 & 313.00 (M+1); 333.00 & 335.00 (M+Na) and (S)-methyl 5-bromo-2-(sec-butyl)-2H-indazole-3-carboxylate (192f) (2.5 g, 30.6% yield) as a white solid; 1H NMR (300 MHZ, DMSO-d₆) δ 8.15 (dt, J=1.5, 0.7 Hz, 1H), 7.79 (dt, J=9.1, 0.8 Hz, 1H), 7.46 (ddd, J=9.0, 1.9, 0.8 Hz, 1H), 5.76-5.59 (m, 1H), 3.98 (s, 3H), 2.11-1.77 (m, 2H), 1.53 (d, J=6.6 Hz, 3H), 0.69 (t, J=7.4 Hz, 3H); MS (ES+): 311.05 & 313.00 (M+1).

### Step-2: Preparation of (S)-(5-bromo-1-(sec-butyl)-1H-indazol-3-yl)methanol (192b)

Compound 192b was prepared according to the procedure reported in step-1 of scheme 2 from (S)-methyl 5-bromo-1-(sec-butyl)-1H-indazole-3-carboxylate (192a) (4.0 g, 12.85 mmol) in DCM (75 mL) using DIBAL (1M solution in DCM, 32.1 mL, 32.1 mmol) to afford after workup and purification using method-R, (S)-(5-bromo-1-(sec-butyl)-1H-indazol-3-yl)methanol (192b) (2.770 g, 76% yield) as a light yellow oil. MS (ES+): 283.0/285.0 (M+1).

### Step-3: Preparation of (S)-ethyl 2-(2-((5-bromo-1-(sec-butyl)-1H-indazol-3-yl)methoxy)phenyl)acetate (192c)

Compound 192c was prepared according to the procedure reported in step-2 of scheme 2 from (S)-(5-bromo-1-(sec-butyl)-1H-indazol-3-yl)methanol (192b) (2.650 g, 9.36 mmol) in DCM (50 mL) using PPh₃ (2.70 g, 10.29 mmol), ethyl 2-(2-hydroxyphenyl)acetate (2b) (2.024 g, 11.23 mmol) and a solution of DCAD (3.78 g, 10.29 mmol) in DCM to afford after workup and purification using method-AU. (S)-ethyl 2-(2-((5-bromo-1-(sec-butyl)-1H-indazol-3-yl)methoxy)phenyl)acetate (192c) (1.407 g, 33.8% yield) as a white solid. MS (ES+): 445.0/447.0 (M+1).

### Step-4: Preparation of (S)-ethyl 2-(2-((5-(1-aminoisoquinolin-7-yl)-1-(sec-butyl)-1H-indazol-3-yl)methoxy)phenyl)acetate (192d)

Compound 192d was prepared according to the procedure reported in step-5 of scheme 1 from (S)-ethyl 2-(2-((5-bromo-1-(sec-butyl)-1H-indazol-3-yl)methoxy)phenyl)acetate (192c) (600 mg, 1.347 mmol) in dioxane/MeTHF (20 mL, 1:1) using 1-aminoisoquinolin-7-ylboronic acid (87a) (329 mg, 1.751 mmol), K₃PO₄ (2M aqueous solution, 1.347 mL, 5.39 mmol). PCy₃ (76 mg, 0.269 mmol), Pd₂(dba)₃ (123 mg, 0.135 mmol) and PdCl₂(dppf)-CH₂Cl₂ adduct (110 mg, 0.135 mmol) to afford after workup and purification using method-AE, (S)-ethyl 2-(2-((5-(1-aminoisoquinolin-7-yl)-1-(sec-butyl)-1H-indazol-3-yl)methoxy)phenyl)acetate (192d) (283 mg, 41.3% yield) as a yellow solid. MS (ES+): 509.20 (M+1).

### Step-5: Preparation of (S)-2-(2-((5-(1-aminoisoquinolin-7-yl)-1-(sec-butyl)-1H-indazol-3-yl)methoxy)phenyl)acetic acid (192e)

Compound 192e was prepared according to the procedure reported step-2 of scheme 1 from (S)-ethyl 2-(2-((5-(1-aminoisoquinolin-7-yl)-1-(sec-butyl)-1H-indazol-3-yl)methoxy)phenyl)acetate (192d) (283 mg, 0.556 mmol) in THF (3 mL) using a solution of LiOH·H₂O (70 mg, 1.669 mmol) in water (1 mL) to afford after workup and purification using method-M, (S)-2-(2-((5-(1-aminoisoquinolin-7-yl)-1-(sec-butyl)-1H-indazol-3-yl)methoxy)phenyl)acetic acid (192e) (58.7 mg, 21.95% yield) HCl salt as a white solid; 1H NMR (300 MHz, DMSO-d₆) δ 13.39 (s, 1H, D₂O exchangeable), 12.13 (s, 1H, D₂O exchangeable), 9.21 (s, 2H, D₂O exchangeable), 8.96 (d, J=1.8 Hz, 1H), 8.41 (dd, J=8.5, 1.6 Hz, 1H), 8.30 (d, J=1.6 Hz, 1H), 8.07-7.99 (m, 1H), 7.99-7.88 (m, 2H), 7.69 (d, J=6.9 Hz, 1H), 7.34-7.23 (m, 3H), 7.23-7.16 (m, 1H), 6.95-6.87 (m, 1H), 5.50 (s, 2H), 4.90-4.76 (m, 1H), 3.53 (s, 2H), 2.10-1.79 (m, 2H), 1.53 (d, J=6.6 Hz, 3H), 0.69 (t, J=7.3 Hz, 3H); MS (ES+): 481.2 (M+1); (ES−): 479.1 (M−1); Optical rotation: +20.00 [0.14, MeOH].

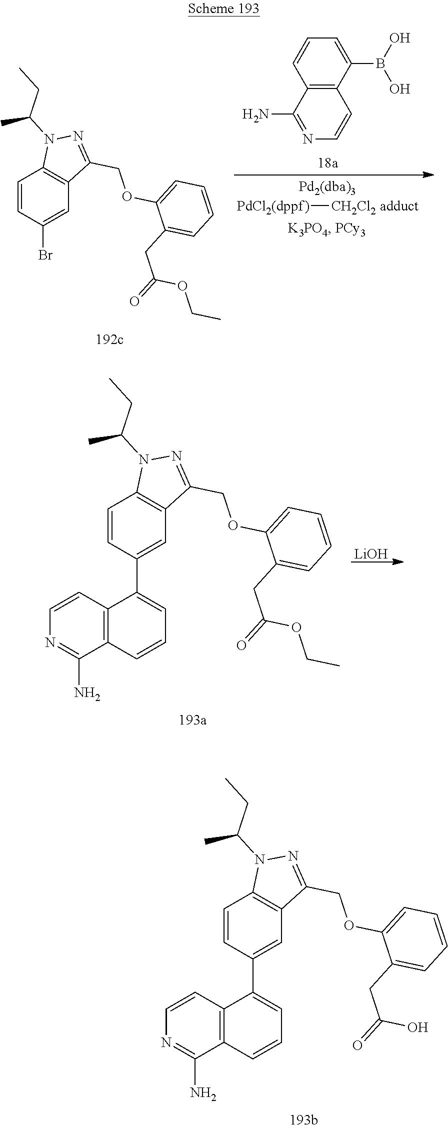

### Preparation of (S)-2-(2-((5-(1-aminoisoquinolin-5-yl)-1-(sec-butyl)-1H-indazol-3-yl)methoxy)phenyl)acetic acid (193b)

### Step-1: Preparation of (S)-ethyl 2-(2-((5-(1-aminoisoquinolin-5-yl)-1-(sec-butyl)-1H-indazol-3-yl)methoxy)phenyl)acetate (193a)

Compound 193a was prepared according to the procedure reported in step-5 of scheme 1 from (S)-ethyl 2-(2-((5-bromo-1-(sec-butyl)-1H-indazol-3-yl)methoxy)phenyl)acetate (192c) (600 mg, 1.347 mmol) in dioxane/MeTHF (25 mL, 9:1) using (1-aminoisoquinolin-5-yl)boronic acid (18a) (253 mg, 1.347 mmol), K₃PO₄ (2M aqueous solution, 1.347 mL, 5.39 mmol), PCy₃ (76 mg, 0.269 mmol), Pd₂(dba)₃ (123 mg, 0.135 mmol) and PdCl₂(dppf)-CH₂Cl₂ adduct (110 mg, 0.135 mmol) to afford after workup and purification using method-AE, (S)-ethyl 2-(2-((5-(1-aminoisoquinolin-5-yl)-1-(sec-butyl)-1H-indazol-3-yl)methoxy)phenyl)acetate (193a) (208 mg, 30.4% yield) as a yellow solid. MS (ES+): 509.30 (M+1).

### Step-2: Preparation of (S)-2-(2-((5-(1-aminoisoquinolin-5-yl)-1-(sec-butyl)-1H-indazol-3-yl)methoxy)phenyl)acetic acid (193b)

Compound 193b was prepared according to the procedure reported step-2 of scheme 1 from (S)-ethyl 2-(2-((5-(1-aminoisoquinolin-5-yl)-1-(sec-butyl)-1H-indazol-3-yl)methoxy)phenyl)acetate (193a) in THF (3 mL) using a solution of LiOH·H₂O (51.5 mg, 1.227 mmol) in water (1 mL) to afford after workup and purification using method-M, (S)-2-(2-((5-(1-aminoisoquinolin-5-yl)-1-(sec-butyl)-1H-indazol-3-yl)methoxy)phenyl)acetic acid (193b) (55 mg, 28% yield) HCl salt as a white solid; 1H NMR (300 MHz, DMSO-d₆) δ 13.43 (s, 1H, D₂O exchangeable), 12.03 (s, 1H, D₂O exchangeable), 9.25 (s, 2H, D₂O exchangeable), 8.63 (d, J=8.3 Hz, 1H), 7.96 (dd, J=7.4, 1.1 Hz, 1H), 7.92-7.81 (m, 3H), 7.62 (d, J=7.2 Hz, 1H), 7.45 (dd, J=8.7, 1.7 Hz, 1H), 7.28-7.20 (m, 2H), 7.20-7.13 (m, 1H), 6.99 (d, J=7.2 Hz, 1H), 6.92-6.84 (m, 1H), 5.46 (s, 2H), 4.92-4.75 (m, 1H), 3.47 (s, 2H), 2.12-1.79 (m, 2H), 1.52 (d, J=6.6 Hz, 3H), 0.72 (t, J=7.3 Hz, 3H); MS (ES+): 481.2 (M+1); (ES−): 479.1 (M−1); Optical rotation: +15.00 [0.12, MeOH].

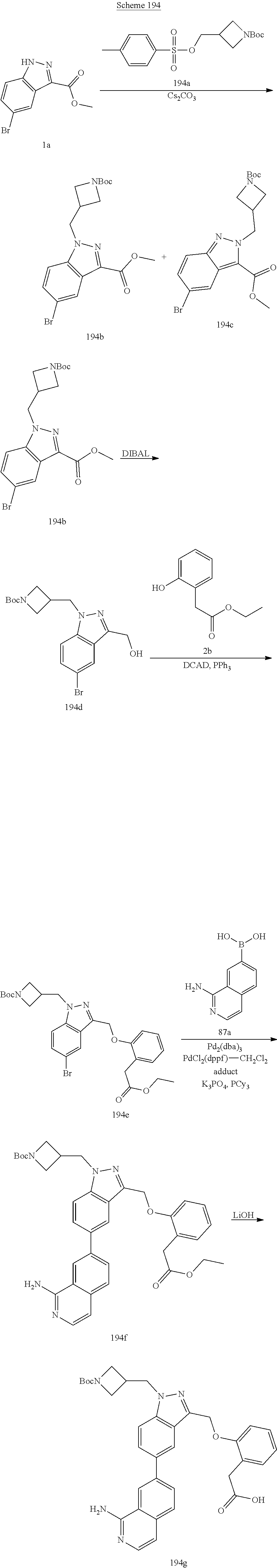

### Preparation of 2-(2-((5-(1-aminoisoquinolin-7-yl)-1-((1-(tert-butoxycarbonyl)azetidin-3-yl)methyl)-1H-indazol-3-yl)methoxy)phenyl)acetic acid (194g)

### Step-1: Preparation of methyl 5-bromo-1-((1-(tert-butoxycarbonyl)azetidin-3-yl)methyl)-1H-indazole-3-carboxylate (194b) and methyl 5-bromo-2-((1-(tert-butoxycarbonyl)azetidin-3-yl)methyl)-2H-indazole-3-carboxylate (194c)

Compounds 194b and 194c were prepared according to the procedure reported in step-2 of scheme 86 from methyl 5-bromo-1H-indazole-3-carboxylate (1a) (13.00 g, 51.0 mmol) in DMF (60 mL) using Cs₂CO₃ (33.2 g, 102 mmol) and tert-butyl 3-((tosyloxy)methyl)azetidine-1-carboxylate (194a) (17.4 g, 51.0 mmol, CAS #892408-42-3) to afford after workup and purification using method-A, methyl 5-bromo-1-((1-(tert-butoxycarbonyl)azetidin-3-yl)methyl)-1H-indazole-3-carboxylate (194b) (8.574 g, 39.7% yield) as a white solid; MS (ES+): 424.1 and 426.1 (M+1), 446.1 and 448.0 (M+Na); and methyl 5-bromo-2-((1-(tert-butoxycarbonyl)azetidin-3-yl)methyl)-2H-indazole-3-carboxylate (194c) (5.27 g, 24.37% yield) as a white solid; MS (ES+): 424.1 and 426.1 (M+1), 446.0 and 448.1 (M+Na).

### Step-2: Preparation of tert-butyl 3-((5-bromo-3-(hydroxymethyl)-1H-indazol-1-yl)methyl)azetidine-1-carboxylate (194d)

Compound 194d was prepared according to the procedure reported in step-1 of scheme 2 from methyl 5-bromo-1-((1-(tert-butoxycarbonyl)azetidin-3-yl)methyl)-1H-indazole-3-carboxylate (194b) (8.574 g, 20.21 mmol) in DCM (75 mL) using DIBAL (1M solution in DCM, 50.5 mL, 50.5 mmol) to afford after workup and purification using method-R, fert-butyl 3-((5-bromo-3-(hydroxymethyl)-1H-indazol-1-yl)methyl)azetidine-1-carboxylate (194d) (4.182 g, 52% yield) as a light yellow oil; MS (ES+): 419.00 (M+Na).

### Step-3: Preparation of tert-butyl 3-((5-bromo-3-((2-(2-ethoxy-2-oxoethyl)phenoxy)methyl)-1H-indazol-1-yl)methyl)azetidine-1-carboxylate (194e)

Compound 194e was prepared according to the procedure reported in step-2 of scheme 2 from tert-butyl 3-((5-bromo-3-(hydroxymethyl)-1H-indazol-1-yl)methyl)azetidine-1-carboxylate (194d) (3.882 g, 9.80 mmol) in DCM (50 mL) using PPh₃ (2.83 g, 10.78 mmol), ethyl 2-(2-hydroxyphenyl)acetate (2b) (2.118 g, 11.76 mmol) and a solution of DCAD (3.96 g, 10.78 mmol) in DCM to afford after workup and purification using method-AV, tert-butyl 3-((5-bromo-3-((2-(2-ethoxy-2-oxoethyl)phenoxy)methyl)-1H-indazol-1-yl)methyl)azetidine-1-carboxylate (194e) (471 mg, 9% yield) as a white solid; MS (ES+): 558.2 (M+1).

### Step-4: Preparation of tert-butyl 3-((5-(1-aminoisoquinolin-7-yl)-3-((2-(2-ethoxy-2-oxoethyl)phenoxy)methyl)-1H-indazol-1-yl)methyl)azetidine-1-carboxylate (194f)

Compound 194f was prepared according to the procedure reported in step-5 of scheme 1 from tert-butyl 3-((5-bromo-3-((2-(2-ethoxy-2-oxoethyl)phenoxy)methyl)-1H-indazol-1-yl)methyl)azetidine-1-carboxylate (194e) (275 mg, 0.492 mmol) in dioxane/MeTHF (10 mL, 1:1) using (1-aminoisoquinolin-7-yl)boronic acid (87a) (120 mg, 0.640 mmol), K₃PO₄ (2M aqueous solution, 0.492 mL, 1.970 mmol), PCy₃ (27.6 mg, 0.098 mmol). Pd₂(dba)₃ (45.1 mg, 0.049 mmol) and PdCl₂(dppf)-CH₂Cl₂ adduct (40.2 mg, 0.049 mmol) to afford after workup and purification using method-AE, tert-butyl 3-((5-(1-aminoisoquinolin-7-yl)-3-((2-(2-ethoxy-2-oxoethyl)phenoxy)methyl)-1H-indazol-1-yl)methyl)azetidine-1-carboxylate (194f) (283 mg, 92% yield) as a yellow solid; (ES+): 622.30 (M+Na).

### Step-5: Preparation of 2-(2-((5-(1-aminoisoquinolin-7-yl)-1-((1-(tert-butoxycarbonyl)azetidin-3-yl)methyl)-1H-indazol-3-yl)methoxy)phenyl)acetic acid (194g)

Compound 194g was prepared according to the procedure reported step-2 of scheme 1 from tert-butyl 3-((5-(1-aminoisoquinolin-7-yl)-3-((2-(2-ethoxy-2-oxoethyl)phenoxy)methyl)-1H-indazol-1-yl)methyl)azetidine-1-carboxylate (194f) (83 mg, 0.133 mmol) in THF (3 mL) using a solution of LiOH·H₂O (16.81 mg, 0.400 mmol) in water (1 mL) to afford after workup and purification using method-M, 2-(2-((5-(1-aminoisoquinolin-7-yl)-1-((1-(tert-butoxycarbonyl)azetidin-3-yl)methyl)-1H-indazol-3-yl)methoxy)phenyl)acetic acid (194g) (31.2 mg, 39.4% yield) HCl salt as a white solid; ¹H NMR (300 MHz, DMSO-d₆) δ 13.20 (s, 1H, D₂O exchangeable), 12.12 (s, 1H, D₂O exchangeable), 9.11 (s, 2H, D₂O exchangeable), 8.94 (s, 1H), 8.46-8.37 (m, 1H), 8.31 (s, 1H), 8.07-7.95 (m, 3H), 7.69 (d, J=6.9 Hz, 1H), 7.31-7.24 (m, 3H), 7.24-7.16 (m, 1H), 6.96-6.87 (m, 1H), 5.48 (s, 2H), 4.71 (d, J=7.2 Hz, 2H), 3.90 (t, J=8.4 Hz, 2H), 3.75 (s, 2H), 3.52 (s, 2H), 3.20-3.01 (m, 1H), 1.37 (s, 9H); MS (ES+): 594.3 (M+1); (ES−): 592.2 (M−1).

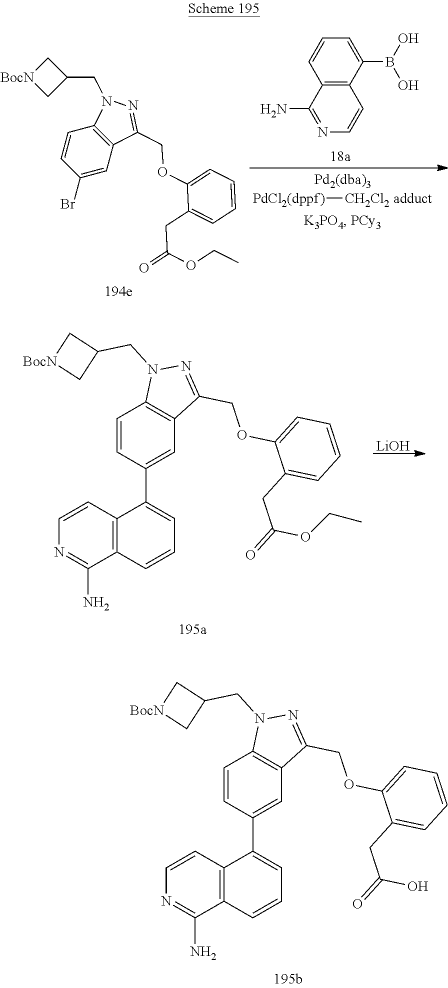

### Preparation of 2-(2-((5-(1-aminoisoquinolin-5-yl)-1-((1-(tert-butoxycarbonyl)azetidin-3-yl)methyl)-1H-indazol-3-yl)methoxy)phenyl)acetic acid (195b)

### Step-1: Preparation of ter-butyl 3-((5-(1-aminoisoquinolin-5-yl)-3-((2-(2-ethoxy-2-oxoethyl)phenoxy)methyl)-1H-indazol-1-yl)methyl)azetidine-1-carboxylate (195a)

Compound 195a was prepared according to the procedure reported in step-5 of scheme 1 from tert-butyl 3-((5-bromo-3-((2-(2-ethoxy-2-oxoethyl)phenoxy)methyl)-1H-indazol-1-yl)methyl)azetidine-1-carboxylate (194e) (275 mg, 0.492 mmol) in dioxane/MeTHF (25 mL, 9:1) using (1-aminoisoquinolin-5-yl)boronic acid (18a) (93 mg, 0.492 mmol), K₃PO₄ (2M aqueous solution, 0.492 mL, 1.970 mmol), PCy₃ (27.6 mg, 0.098 mmol), Pd₂(dba)₃ (45.1 mg, 0.049 mmol) and PdCl₂(dppf)-CH₂Cl₂ adduct (40.2 mg, 0.049 mmol) to afford after workup and purification using method-AE, tert-butyl 3-((5-(1-aminoisoquinolin-5-yl)-3-((2-(2-ethoxy-2-oxoethyl)phenoxy)methyl)-1H-indazol-1-yl)methyl)azetidine-1-carboxylate (195a) (92 mg, 30.1%) as a yellow solid; MS (ES+): 622.3 (M+1).

### Step-2: Preparation of 2-(2-((5-(1-aminoisoquinolin-5-yl)-1-((1-(tert-butoxycarbonyl)azetidin-3-yl)methyl)-1H-indazol-3-yl)methoxy)phenyl)acetic acid (195b)

Compound 195b was prepared according to the procedure reported step-2 of scheme 1 from tert-butyl 3-((5-(1-aminoisoquinolin-5-yl)-3-((2-(2-ethoxy-2-oxoethyl)phenoxy)methyl)-1H-indazol-1-yl)methyl)azetidine-1-carboxylate (195a) (92 mg, 0.148 mmol) in THF (3 mL) using a solution of LiOH·H₂O (18.63 mg, 0.444 mmol) in water (1 mL) to afford after workup and purification using method-M, 2-(2-((5-(1-aminoisoquinolin-5-yl)-1-((1-(tert-butoxycarbonyl)azetidin-3-yl)methyl)-1H-indazol-3-yl)methoxy)phenyl)acetic acid (195b) (43.7 mg, 49.7% yield) HCl salt as a white solid; ¹H NMR (300 MHz, DMSO-d₆) δ 13.23 (s, 1H, D₂O exchangeable), 12.02 (s, 1H, D₂O exchangeable), 9.12 (s, 2H, D₂O exchangeable), 8.61 (d, J=8.3 Hz, 1H), 7.95 (t, J=7.3 Hz, 2H), 7.90-7.81 (m, 2H), 7.62 (d, J=7.3 Hz, 1H), 7.53-7.47 (m, 1H), 7.27-7.20 (m, 2H), 7.20-7.13 (m, 1H), 6.98 (d, J=7.2 Hz, 1H), 6.94-6.86 (m, 1H), 5.45 (s, 2H), 4.71 (d, J=7.2 Hz, 2H), 4.00-3.85 (m, 2H), 3.77 (s, 2H), 3.47 (s, 2H), 3.18-3.05 (m, 1H), 1.38 (s, 9H); MS (ES+): 594.3 (M+1); (ES−): 592.3 (M−1).

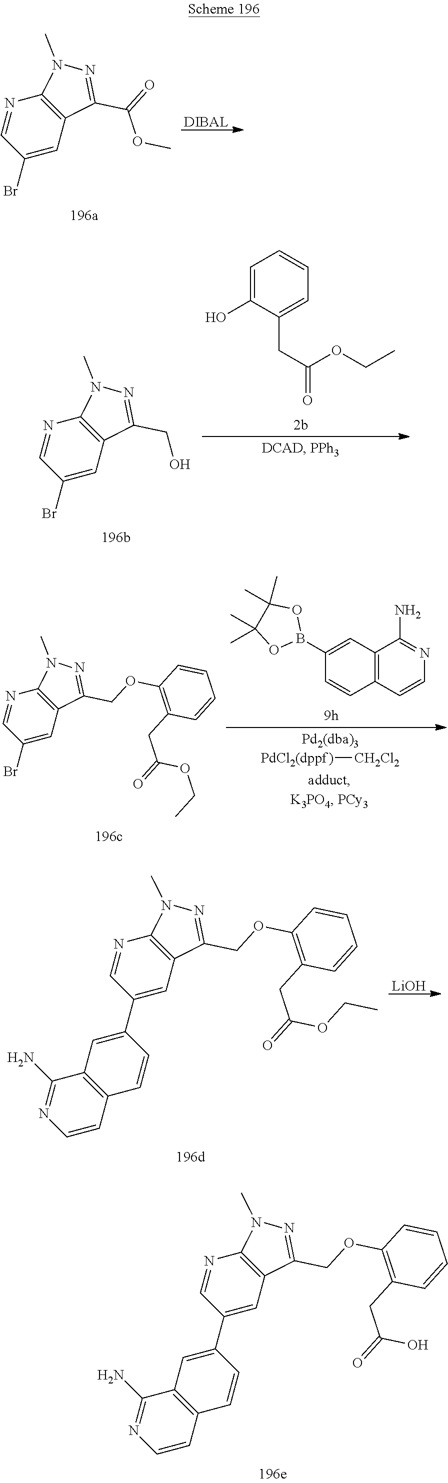

### Preparation of 2-(2-((5-(1-aminoisoquinolin-7-yl)-1-methyl-1H-pyrazolo[3,4-b]pyridin-3-yl)methoxy)phenyl)acetic acid (196e)

### Step-1: Preparation of (5-bromo-1-methyl-1H-pyrazolo[3,4-b]pyridin-3-yl)methanol (196b)

Compound 196b was prepared according to the procedure reported in step-1 of scheme 2 from methyl 5-bromo-1-methyl-1H-pyrazolo[3,4-b]pyridine-3-carboxylate (196a) (600 mg, 2.222 mmol; CAS #: 916326-80-2) in DCM (20 mL) using DIBAL (1M solution in Dichloromethane, 5.55 mL, 5.55 mmol) to afford after workup and purification using method-J, (5-bromo-1-methyl-1H-pyrazolo[3,4-b]pyridin-3-yl)methanol (196b) (400 mg, 74.4% yield) as a white solid; ¹H NMR (300 MHz, DMSO-d₆) δ 8.62 (d, J=2.2 Hz, 1H), 8.55 (d, J=2.2 Hz, 1H), 5.48-5.37 (m, 1H), 4.74 (d, J=4.9 Hz, 2H), 4.00 (s, 3H); MS (ES+): 242.00 & 244.00 (M+1).

### Step-2: Preparation of ethyl 2-(2-((5-bromo-1-methyl-1H-pyrazolo[3,4-b]pyridin-3-yl)methoxy)phenyl)acetate (196c)

Compound 196c was prepared according to the procedure reported in step-2 of scheme 2 from (5-bromo-1-methyl-1H-pyrazolo[3,4-b]pyridin-3-yl)methanol (196b) (400 mg, 1.652 mmol) in DCM (50 mL) using PPb₃ (520 mg, 1.983 mmol), ethyl 2-(2-hydroxyphenyl)acetate (2b) (328 mg, 1.818 mmol) and a solution of DCAD (728 mg, 1.983 mmol) in DCM (10 mL) to afford after workup and purification using method-N, ethyl 2-(2-((5-bromo-1-methyl-1H-pyrazolo[3,4-b]pyridin-3-yl)methoxy)phenyl)acetate (196c) (450 mg, 67.4% yield) as a white oil; 1H NMR (300 MHz, DMSO-d₆) δ 8.67 (d, J=2.2 Hz, 1H), 8.48 (d, J=2.2 Hz, 1H), 7.29 (ddd, J=8.7, 7.2, 1.7 Hz, 1H), 7.21 (ddd, J=7.4, 3.3, 1.4 Hz, 2H), 6.93 (td, J=7.3, 1.3 Hz, 1H), 5.37 (s, 2H), 4.06 (s, 3H), 3.89 (q. J=7.1 Hz, 2H), 3.54 (s, 2H), 0.94 (t, J=7.1 Hz, 3H); MS (ES+): 404.10 & 406.10 (M+1); 426.00 & 428.00 (M+Na); MS (ES−): 402.10 & 404.00 (M−1).

### Step-3: Preparation of ethyl 2-(2-((5-(1-aminoisoquinolin-7-yl)-1-methyl-1H-pyrazolo[3,4-b]pyridin-3-yl)methoxy)phenyl)acetate (196d)

Compound 196d was prepared according to the procedure reported in step-5 of scheme 1 from ethyl 2-(2-((5-bromo-1-methyl-1H-pyrazolo[3,4-b]pyridin-3-yl)methoxy)phenyl)acetate (196c) (220 mg, 0.544 mmol) in dioxane/THF (4 mL, 1:1) using 7-(4,4,5,5-tetramethyl-1,3,2-dioxaborolan-2-yl)isoquinolin-1-amine (9h) (221 mg, 0.816 mmol). K₃PO₄ (2M aqueous solution, 1.088 mL, 2.177 mmol), PCy₃ (30.5 mg, 0.109 mmol), PdCl₂(dppf)-CH₂Cl₂ adduct (44.4 mg, 0.054 mmol) and Pd₂(dba)₃ (49.8 mg, 0.054 mmol) to afford after workup and purification using method-AY, ethyl 2-(2-((5-(1-aminoisoquinolin-7-yl)-1-methyl-1H-pyrazolo[3,4-b]pyridin-3-yl)methoxy)phenyl)acetate (196d) (80 mg, 31.4% yield) as a clear oil; 1H NMR (300 MHz, DMSO-d₆) δ 9.12 (d, J=2.1 Hz, 1H), 8.63 (d, J=2.2 Hz, 2H), 8.09 (d, J=8.3 Hz, 1H), 7.87-7.78 (m, 2H), 7.34-7.23 (m, 2H), 7.20 (d, J=7.4 Hz, 1H), 7.03 (s, 2H), 6.99-6.88 (m, 2H), 5.45 (s, 2H), 4.12 (s, 3H), 3.63 (q, J=7.1 Hz, 2H), 3.55 (s, 2H), 0.72 (t, J=7.1 Hz, 3H); MS (ES+): 468.15 (M+1).

### Step-4: Preparation of 2-(2-((5-(1-aminoisoquinolin-7-yl)-1-methyl-1H-pyrazolo[3,4-b]pyridin-3-yl)methoxy)phenyl)acetic acid (196e)

Compound 196e was prepared according to the procedure reported step-2 of scheme 1 from ethyl 2-(2-((5-(1-aminoisoquinolin-7-yl)-1-methyl-1H-pyrazolo[3,4-b]pyridin-3-yl)methoxy)phenyl)acetate (196d) (80 mg, 0.171 mmol) in THF/MeOH (4 mL) using a solution of LiOH·H₂O (43.1 mg, 1.027 mmol) in water (1 mL) to afford after workup and purification using method-G, 2-(2-((5-(1-aminoisoquinolin-7-yl)-1-methyl-1H-pyrazolo[3,4-b]pyridin-3-yl)methoxy)phenyl)acetic acid (196e) (32 mg, 42.6% yield) HCl salt as a white solid; ¹H NMR (300 MHz, DMSO-d₆) δ 13.39 (s, 1H, D₂O exchangeable), 9.18 (s, 2H, D₂O exchangeable), 9.16 (d, J=2.1 Hz, 1H), 9.06 (s, 1H), 8.76 (d, J=2.2 Hz, 1H), 8.45 (dd, J=8.5, 1.6 Hz, 1H), 8.09 (d, J=8.5 Hz, 1H), 7.71 (d, J=7.0 Hz, 1H), 7.34-7.24 (m, 3H), 7.22 (d, 1H), 6.98-6.87 (m, 1H), 5.47 (s, 2H), 4.13 (s, 3H), 3.54 (s, 2H); MS (ES+): 440.15 (M+1); (ES−): 438.10 (M−1).

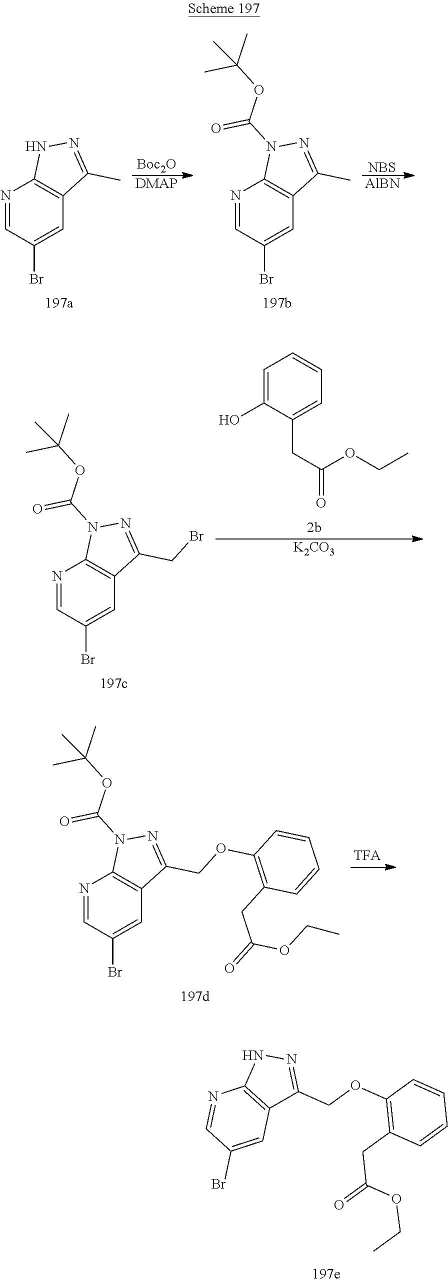

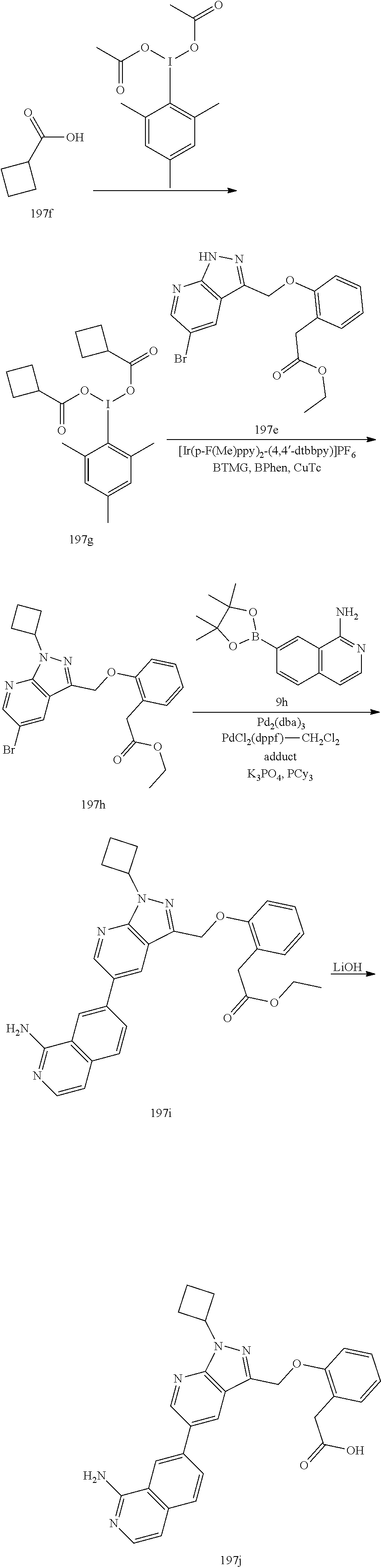

### Preparation of 2-(2-((5-(1-aminoisoquinolin-7-yl)-1-cyclobutyl-1H-pyrazolo[3,4-b]pyridin-3-yl)methoxyphenyl)acetic acid (197j)

### Step-1: Preparation of ter-butyl 5-bromo-3-methyl-1H-pyrazolo[3,4-b]pyridine-1-carboxylate (197b)

To a solution of 5-bromo-3-methyl-1H-pyrazolo[3,4-b]pyridine (197a) (3 g, 14.15 mmol; CAS #: 885223-65-4) in acetonitrile (60 mL) was added Boc-Anhydride (3.28 mL, 14.15 mmol), DMAP (1.728 g, 14.15 mmol) and stirred at room temperature for 15 h. The mixture was poured into EtOAc and washed with water and brine. The organic layer was dried, filtered and concentrated in vacuum. The obtained residue was purified using method-I to give tert-butyl 5-bromo-3-methyl-1H-pyrazolo[3,4-b]pyridine-1-carboxylate (197b) (4 g, 91% yield) as a white solid; ¹H NMR (300 MHz, DMSO-d₆) δ 8.76 (d, J=2.3 Hz, 1H), 8.68 (d, J=2.2 Hz, 1H), 2.51 (s, 3H), 1.61 (s, 9H); MS (ES+): 334.00 & 336.00 (M+Na).

### Step-2: Preparation of tert-butyl 5-bromo-3-(bromomethyl)-1H-pyrazolo[3,4-b]pyridine-1-carboxylate (197c)

A mixture of tert-butyl 5-bromo-3-methyl-1H-pyrazolo[3,4-b]pyridine-1-carboxylate (197b) (2 g, 6.41 mmol), NBS (1.254 g, 7.05 mmol), AIBN (0.105 g, 0.641 mmol) in CCl₄ (30 mL) was heated at reflux for 4 h. The mixture was cooled to room temperature, filtered and diluted with EtOAc. The organic layer was washed with water, brine, dried, filtered, and concentrated in vacuum. The obtained residue was purified using method-I to give tert-butyl 5-bromo-3-(bromomethyl)-1H-pyrazolo[3,4-b]pyridine-1-carboxylate (197c) (1.25 g, 49.9% yield) as a white solid; 1H NMR (300 MHz, DMSO-d₆) δ 8.84 (d, J=2.2 Hz, 1H), 8.77 (d, J=2.2 Hz, 1H), 5.03 (s, 2H), 1.63 (s, 9H); MS (ES+): 289.80 & 291.85 (M-Boc+1).

### Step-3: Preparation of tert-butyl 5-bromo-3-((2-(2-ethoxy-2-oxoethyl)phenoxy)methyl)-1H-pyrazolo[3,4-b]pyridine-1-carboxy late (197d)

Compound 197d was prepared according to the procedure reported in step-3 of scheme 106 from tert-butyl 5-bromo-3-(bromomethyl)-1H-pyrazolo[3,4-b]pyridine-1-carboxylate (197c) (1.2 g, 3.07 mmol) in DMF (30 mL) using K₂CO₃ (1.27 g, 9.21 mmol) and ethyl 2-(2-hydroxyphenyl)acetate (2b) (0.664 g, 3.68 mmol) to afford after workup and purification using method-BB, tert-butyl 5-bromo-3-((2-(2-ethoxy-2-oxoethyl)phenoxy)methyl)-1H-pyrazolo[3,4-b]pyridine-1-carboxy late (197d) (500 mg, 33.2% yield) as a light yellow solid; 1H NMR (300 MHz, DMSO-d₆) δ 8.84 (d, J=2.3 Hz, 1H), 8.59 (d, J=2.2 Hz, 1H), 7.31 (td, J=7.8, 7.3, 1.7 Hz, 1H), 7.22 (ddd, J=7.7, 5.2, 3.0 Hz, 2H), 6.96 (td, J=7.4, 1.1 Hz, 1H), 5.43 (s, 2H), 3.89 (q. J=7.1 Hz, 2H), 3.57 (s, 2H), 1.63 (s, 9H), 0.94 (t, J=7.1 Hz, 3H); MS (ES+): 390.05 & 392.10 (M-Boc+1); 512.10 & 514.10 (M+Na).

### Step-4: Preparation of ethyl 2-(2-((5-bromo-1H-pyrazolo[3,4-b]pyridin-3-yl)methoxy)phenyl)acetate (197e)

Compound 197e was prepared according to the procedure reported in step-4 of scheme 9 from tert-butyl 5-bromo-3-((2-(2-ethoxy-2-oxoethyl)phenoxy)methyl)-1H-pyrazolo[3,4-b]pyridine-1-carboxylate (197d) (550 mg, 1.122 mmol) in DCM (20 mL) using TFA (0.86 mL, 11.22 mmol) to afford after workup and purification using method-T, ethyl 2-(2-((5-bromo-1H-pyrazolo[3,4-b]pyridin-3-yl)methoxy)phenyl)acetate (197e) (438 mg, 100% yield) as a yellow solid; MS (ES+): 390.00 & 392.00 (M+1); 412.00 & 414.00 (M+Na); (ES−): 388.00 & 390.00 (M−1).

### Step-5: Preparation of iodomesitylene bis(cyclobutanecarboxylate) (197g)

Compound 197g was prepared according to the procedure reported in step-5 of scheme 9, from Iodomesitylene diacetate (2 g, 5.49 mmol; CAS #33035-41-5) in toluene (40 mL) using cyclobutanecarboxylic acid (197f) (1.1 g, 10.98 mmol; CAS #3721-95-7) to afford after workup iodomesitylene bis(cyclobutanecarboxylate) (197g) (2.440 g, 100% yield), which was used the next step without further purification.

### Step-6: Preparation of ethyl 2-(2-((5-bromo-1-cyclobutyl-1H-pyrazolo[3,4-b]pyridin-3-yl)methoxy)phenyl)acetate (197h)

Compound 197h was prepared according to the procedure reported in step-6 of scheme 9 from ethyl 2-(2-((5-bromo-1H-pyrazolo[3,4-b]pyridin-3-yl)methoxy)phenyl)acetate (197e) (450 mg, 1.153 mmol) in 1,4-dioxane (10 mL) using iodomesitylene bis(cyclobutanecarboxylate) (197g) (1025 mg, 2.306 mmol), [Ir(p-F(Me)ppy)2-(4,4′-dtbbpy)]PF₆ (11.28 mg, 0.012 mmol), 2-tert-butyl-1,1,3,3-tetramethylguanidine (BTMG, 395 mg, 2.306 mmol), 4,7-diphenyl-1,10-phenanthroline (BPhen, 115 mg, 0.346 mmol) and copper(I) thiophene-2-carboxylate (CuTC) (44 mg, 0.231 mmol) to afford after workup and purification using method-J, ethyl 2-(2-((5-bromo-1-cyclobutyl-1H-pyrazolo[3,4-b]pyridin-3-yl)methoxy)phenyl)acetate (197h) (200 mg, 39.0% yield) as a clear oil; MS (ES+): 444.10 & 446.10 (M+1).

### Step-7: Preparation of ethyl 2-(2-((5-(1-aminoisoquinolin-7-yl)-1-cyclobutyl-1H-pyrazolo[3,4-b]pyridin-3-yl)methoxy)phenyl)acetate (197i)

Compound 197i was prepared according to the procedure and method of purification reported in step-5 of scheme 1 from ethyl 2-(2-((5-bromo-1-cyclobutyl-1H-pyrazolo[3,4-b]pyridin-3-yl)methoxy)phenyl)acetate (197h) (100 mg, 0.225 mmol) in dioxane/THF (4 mL, 1:1) using 7-(4,4,5,5-tetramethyl-1,3,2-dioxaborolan-2-yl)isoquinolin-1-amine (9h) (73.0 mg, 0.27 mmol), K₃PO₄ (2M aqueous solution, 0.450 mL, 0.900 mmol), PCy₃ (12.62 mg, 0.045 mmol), PdCl₂(dppf)-CH₂Cl₂ adduct (18.38 mg, 0.023 mmol) and Pd₂(dba)₃ (20.61 mg, 0.023 mmol) to afford after workup and purification using method-Y, ethyl 2-(2-((5-(1-aminoisoquinolin-7-yl)-1-cyclobutyl-1H-pyrazolo[3,4-b]pyridin-3-yl)methoxy)phenyl)acetate (197i) (80 mg, 70.0% yield) as a clear oil; MS (ES+): 508.20 (M+1).

### Step-8: Preparation of 2-(2-((5-(1-aminoisoquinolin-7-yl)-1-cyclobutyl-1H-pyrazolo[3,4-b]pyridin-3-yl)methoxy)phenyl)acetic acid (197j)

Compound 197j was prepared according to the procedure reported in step-2 of scheme 1 from ethyl 2-(2-((5-(1-aminoisoquinolin-7-yl)-1-cyclobutyl-1H-pyrazolo[3,4-b]pyridin-3-yl)methoxy)phenyl)acetate (197i) (80 mg, 0.158 mmol) in MeOH/THF (6 mL, 1:1) using a solution of LiOH·H₂O (39.7 mg, 0.946 mmol) in water (I mL) to afford after workup and purification using method-G, 2-(2-((5-(1-aminoisoquinolin-7-yl)-1-cyclobutyl-1H-pyrazolo[3,4-b]pyridin-3-yl)methoxy)phenyl)acetic acid (197j) (32 mg, 42.3% yield) HCl salt as a white solid; ¹H NMR (300 MHz, DMSO-d₆) δ 13.59 (s, 1H, D₂O exchangeable), 9.32 (s, 2H, D₂O exchangeable), 9.15 (d, J=2.1 Hz, 1H), 9.10 (s, 1H), 8.75 (d, J=2.1 Hz, 1H), 8.44 (dd, J=8.5, 1.6 Hz, 1H), 8.07 (d, J=8.5 Hz, 1H), 7.71 (d, J=7.0 Hz, 1H), 7.34-7.24 (m, 3H), 7.21 (d, J=7.3 Hz, 1H), 6.99-6.86 (m, 1H), 5.59-5.51 (m, 1H), 5.50 (s, 2H), 3.55 (s, 2H), 2.85-2.64 (m, 2H), 2.59-2.50 (m, 2H), 2.01-1.84 (m, 2H); MS (ES+): 480.20 (M+1); (ES−): 478.10 (M−1).

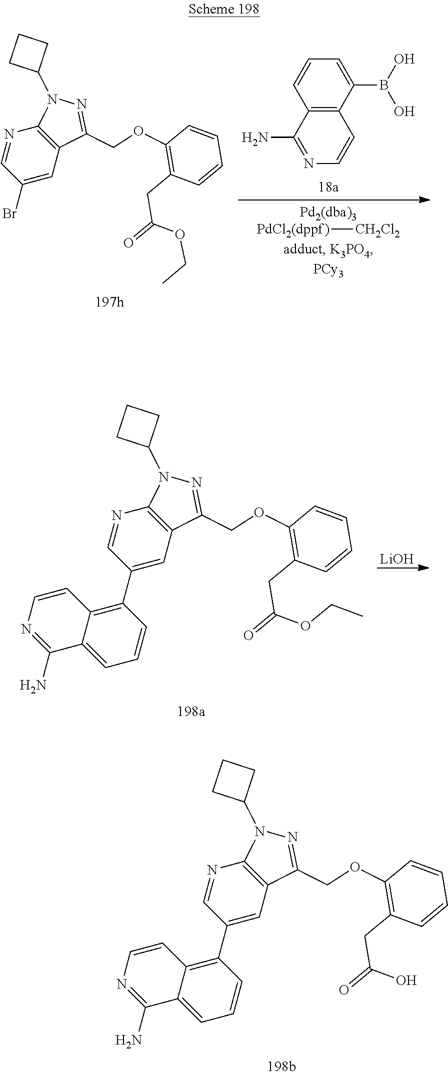

### Preparation of 2-(2-((5-(1-aminoisoquinolin-5-yl)-1-cyclobutyl-1H-pyrazolo[3,4-b]pyridin-3-yl)methoxy)phenyl)acetic acid (198b)

### Step-1: Preparation of ethyl 2-(2-((5-(1-aminoisoquinolin-5-yl)-1-cyclobutyl-1H-pyrazolo[3,4-b]pyridin-3-yl)methoxy)phenyl)acetate (198a)

Compound 198a was prepared according to the procedure and method of purification reported in step-5 of scheme 1 from ethyl 2-(2-((5-bromo-1-cyclobutyl-1H-pyrazolo[3,4-b]pyridin-3-yl)methoxy)phenyl)acetate (197h) (100 mg, 0.225 mmol) in dioxane/THF (4 ml, 1:1) using (1-aminoisoquinolin-5-yl)boronic acid (18a) (63.5 mg, 0.338 mmol), PdCl₂(dppf)-CH₂Cl₂ adduct (18.38 mg, 0.023 mmol), K₃PO₄ (2M aqueous solution, 0.450 mL, 0.900 mmol), PCy₃ (12.62 mg, 0.045 mmol) and Pd₂(dba)₃ (20.61 mg, 0.023 mmol) to afford after workup and purification using method-Y, ethyl 2-(2-((5-(1-aminoisoquinolin-5-yl)-1-cyclobutyl-1H-pyrazolo[3,4-b]pyridin-3-yl)methoxy)phenyl)acetate (198a) (80 mg, 070.0% yield) as a clear oil; MS (ES+): 508.20 (M+1).

### Step-2: Preparation of 2-(2-((5-(1-aminoisoquinolin-5-yl)-1-cyclobutyl-1H-pyrazolo[3,4-b]pyridin-3-yl)methoxy)phenyl)acetic acid (198b)

Compound 198b was prepared according to the procedure reported in step-2 of scheme 1 from ethyl 2-(2-((5-(1-aminoisoquinolin-5-yl)-1-cyclobutyl-1H-pyrazolo[3,4-b]pyridin-3-yl)methoxy)phenyl)acetate (198a) (80 mg, 0.158 mmol) in MeOH/THF (6 mL, 1:1) using a solution of LiOH·H₂O (39.7 mg, 0.946 mmol) in water (2 mL) to afford after workup and purification using method-G, 2-(2-((5-(1-aminoisoquinolin-5-yl)-1-cyclobutyl-1H-pyrazolo[3,4-b]pyridin-3-yl)methoxy)phenyl)acetic acid (198b) (20 mg, 26.5% yield) HCl salt as a white solid; 1H NMR (300 MHz, DMSO-d₆) δ 13.34 (s, 1H, D₂O exchangeable), 9.25 (s, 2H, D₂O exchangeable), 8.68 (d, J=8.4 Hz, 1H), 8.63 (d, J=2.0 Hz, 1H), 8.38 (d, J=2.1 Hz, 1H), 8.03 (d, J=7.2 Hz, 1H), 7.89 (t, J=7.8 Hz, 1H), 7.62 (d, J=7.2 Hz, 1H), 7.31-7.22 (m, 2H), 7.19 (d, J=7.3 Hz, 1H), 6.97 (d, J=7.2 Hz, 1H), 6.95-6.88 (m, 1H), 5.64-5.51 (m, 1H), 5.49 (s, 2H), 3.49 (s, 2H), 2.84-2.69 (m, 2H), 2.57-2.51 (m, 2H), 2.00-1.87 (m, 2H); MS (ES+): 480.20 (M+1); (ES−): 478.10 (M−1).

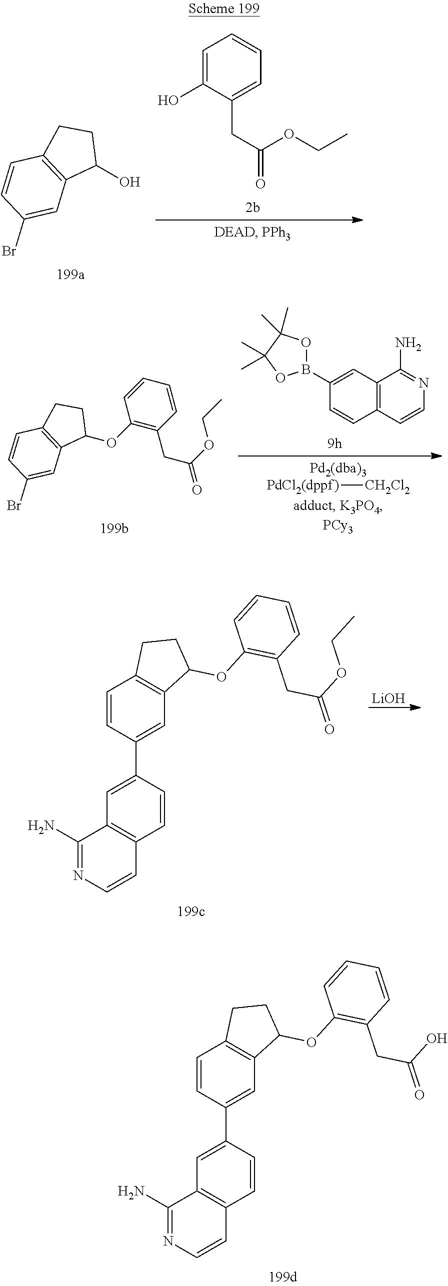

### Preparation of 2-(2-((6-(1-aminoisoquinolin-7-yl)-2,3-dihydro-1H-inden-1-yl)oxy)phenyl)acetic acid (199d)

### Step-1: Preparation of ethyl 2-(2-((6-bromo-2,3-dihydro-1H-inden-1-yl)oxy)phenyl)acetate (199b)

Compound 199b was prepared according to the procedure reported in step-1 of scheme 8 from 6-bromo-2,3-dihydro-1H-inden-1-ol (199a) (400 mg, 1.877 mmol; CAS #75476-86-7) in DCM (10 mL) using ethyl 2-(2-hydroxyphenyl)acetate (2b) (372 mg, 2.065 mmol), PPh₃ (591 mg, 2.253 mmol) and a solution of DEAD (392 mg, 2.253 mmol) in DCM (10 mL) to afford after workup and purification using method-P, ethyl 2-(2-((6-bromo-2,3-dihydro-1H-inden-1-yl)oxy)phenyl)acetate (199b) (380 mg, 53.9% yield) as a white oil; MS (ES+): 397.00 & 399.00 (M+Na); (ES−): 373.00 & 375.00 (M−1).

### Step-2: Preparation of ethyl 2-(2-((6-(1-aminoisoquinolin-7-yl)-2,3-dihydro-1H-inden-1-yl)oxy)phenyl)acetate (199c)

Compound 199c was prepared according to the procedure reported in step-5 of scheme 1 from ethyl 2-(2-((6-bromo-2,3-dihydro-1H-inden-1-yl)oxy)phenyl)acetate (199b) (190 mg, 0.506 mmol) in dioxane/THF (4 mL, 1:1) using 7-(4,4,5,5-tetramethyl-1,3,2-dioxaborolan-2-yl)isoquinolin-1-amine (9h) (205 mg, 0.759 mmol), K₃PO₄ (2M aqueous solution, 1.013 mL, 2.025 mmol), PCy₃ (28.4 mg, 0.101 mmol), PdCl₂(dppf)-CH₂Cl₂ adduct (41.3 mg, 0.051 mmol) and Pd₂(dba)₃ (46.4 mg, 0.051 mmol) to afford after workup and purification using method-Y, ethyl 2-(2-((6-(1-aminoisoquinolin-7-yl)-2,3-dihydro-1H-inden-1-yl)oxy)phenyl)acetate (199c) (120 mg, 54.0% yield) as a clear oil; MS (ES+): 439.20 (M+1).

### Step-3: Preparation of 2-(2-((6-(1-aminoisoquinolin-7-yl)-2,3-dihydro-1H-inden-1-yl)oxy)phenyl)acetic acid (199d)

Compound 199d was prepared according to the procedure reported step-2 of scheme 1 from ethyl 2-(2-((6-(1-aminoisoquinolin-7-yl)-2,3-dihydro-1H-inden-1-yl)oxy)phenyl)acetate (199c) (120 mg, 0.274 mmol) in MeOH/THF (6 mL, 1:1) using a solution of LiOH·H₂O (68.9 mg, 1.642 mmol) in water (2 mL) to afford after workup and purification using method-G, 2-(2-((6-(1-aminoisoquinolin-7-yl)-2,3-dihydro-1H-inden-1-yl)oxy)phenyl)acetic acid (199d) (45 mg, 40.1% yield) HCl salt as a white solid; ¹H NMR (300 MHz, DMSO-d₆) δ 13.34 (s, 1H, D₂O exchangeable), 12.15 (s, 1H, D₂O exchangeable), 9.17 (s, 2H, D₂O exchangeable), 8.91 (s, 1H), 8.31 (d, J=8.4 Hz, 1H), 8.03 (d, J=8.5 Hz, 1H), 7.87 (d, J=7.9 Hz, 2H), 7.69 (d, J=6.9 Hz, 1H), 7.51 (d, J=7.9 Hz, 1H), 7.34-7.17 (m, 4H), 6.93 (t, J=7.1 Hz, 1H), 5.91 (t, J=5.7 Hz, 1H), 3.48 (s, 2H), 3.18-3.04 (m, 1H), 3.02-2.87 (m, 1H), 2.77-2.61 (m, 1H), 2.11-1.95 (m, 1H); MS (ES+): 411.10 (M+1); (ES−): 409.10 (M−1); Analysis calculated for C₂₆H₂₂N₂O₃·HCl, 1.25H₂O: C, 66.52; H, 5.48; Cl, 7.55; N, 5.97; Found: C, 66.53; H, 5.41; Cl, 7.29; N, 5.96.

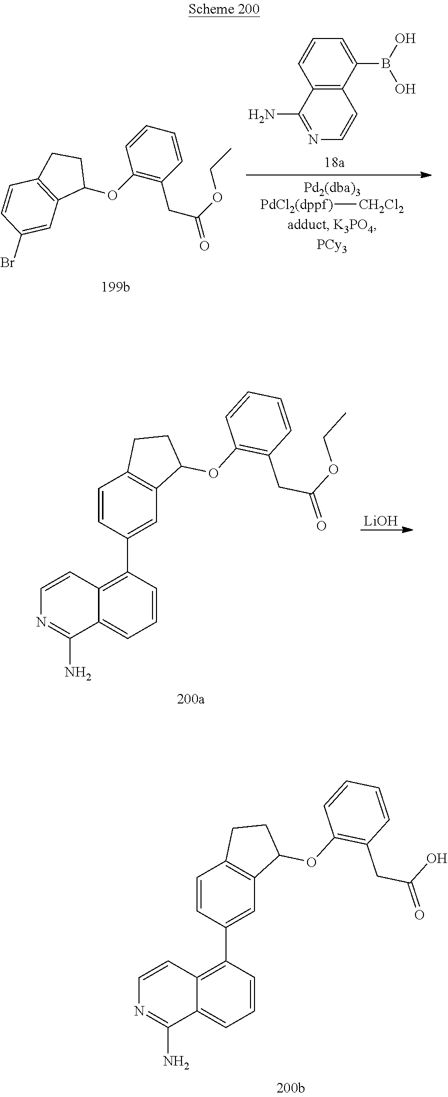

### Preparation of 2-(2-((6-(1-aminoisoquinolin-5-yl)-2,3-dihydro-1H-inden-1-yl)oxy)phenyl)acetic acid (200b)

### Step-1: Preparation of ethyl 2-(2-((6-(1-aminoisoquinolin-5-yl)-2,3-dihydro-1H-inden-1-yl)oxy)phenyl)acetate (200a)

Compound 200a was prepared according to the procedure reported in step-5 of scheme 1 from ethyl 2-(2-((6-bromo-2,3-dihydro-1H-inden-1-yl)oxy)phenyl)acetate (199b) (190 mg, 0.506 mmol) in dioxane/THF (4 mL, 1:1) using (1-aminoisoquinolin-5-yl)boronic acid (18a) (143 mg, 0.759 mmol), K₃PO₄ (2 M aqueous solution, 1.013 mL, 2.025 mmol), PCy₃ (28.4 mg, 0.101 mmol), PdCl₂(dppf)-CH₂Cl₂ adduct (41.3 mg, 0.051 mmol) and Pd₂(dba)₃ (46.4 mg, 0.051 mmol) to afford after workup and purification using method-Y, ethyl 2-(2-((6-(1-aminoisoquinolin-5-yl)-2,3-dihydro-1H-inden-1-yl)oxy)phenyl)acetate (200a) (120 mg, 54.0% yield) as a clear oil; MS (ES+): 439.20 (M+1).

### Step-2: Preparation of 2-(2-((6-(1-aminoisoquinolin-5-yl)-2,3-dihydro-1H-inden-1-yl)oxy)phenyl)acetic acid (200b)

Compound 200b was prepared according to the procedure reported in step-2 of scheme 1 from ethyl 2-(2-((6-(1-aminoisoquinolin-5-yl)-2,3-dihydro-1H-inden-1-yl)oxy)phenyl)acetate (200a) (120 mg, 0.274 mmol) in MeOH/THF (6 mL, 1:1) using a solution of LiOH·H₂O (68.9 mg, 1.642 mmol) in water (2 mL) to afford after workup and purification using method-G, 2-(2-((6-(1-aminoisoquinolin-5-yl)-2,3-dihydro-1H-inden-1-yl)oxy)phenyl)acetic acid (200b) (30 mg, 26.7% yield) HCl salt as a white solid; 3H NMR (300 MHz, DMSO-d₆) δ 13.16 (s, 1H, D₂O exchangeable), 12.01 (s, 1H, D₂O exchangeable), 9.10 (s, 2H, D₂O exchangeable), 8.59 (d, J=8.0 Hz, 1H), 7.94-7.77 (m, 2H), 7.60 (d, J=7.3 Hz, 1H), 7.51 (d, J=7.9 Hz, 1H), 7.38 (d, J=7.4 Hz, 2H), 7.30-7.22 (m, 2H), 7.19 (d, J=7.8 Hz, 1H), 6.99-6.86 (m, 2H), 5.90 (t, J=5.9 Hz, 1H), 3.44 (d, J=3.3 Hz, 2H), 3.21-3.07 (m, 1H), 3.05-2.91 (m, 1H), 2.79-2.65 (m, 1H), 2.15-1.98 (m, 1H); MS (ES+): 411.15 (M+1); (ES−): 409.10 (M−1); Analysis calculated for C₂₆H₂₂N₂O₃·HCl·2H₂O: C, 64.66; H, 5.64; N, 5.80; Found: C, 64.91; H, 5.43; N, 5.93.

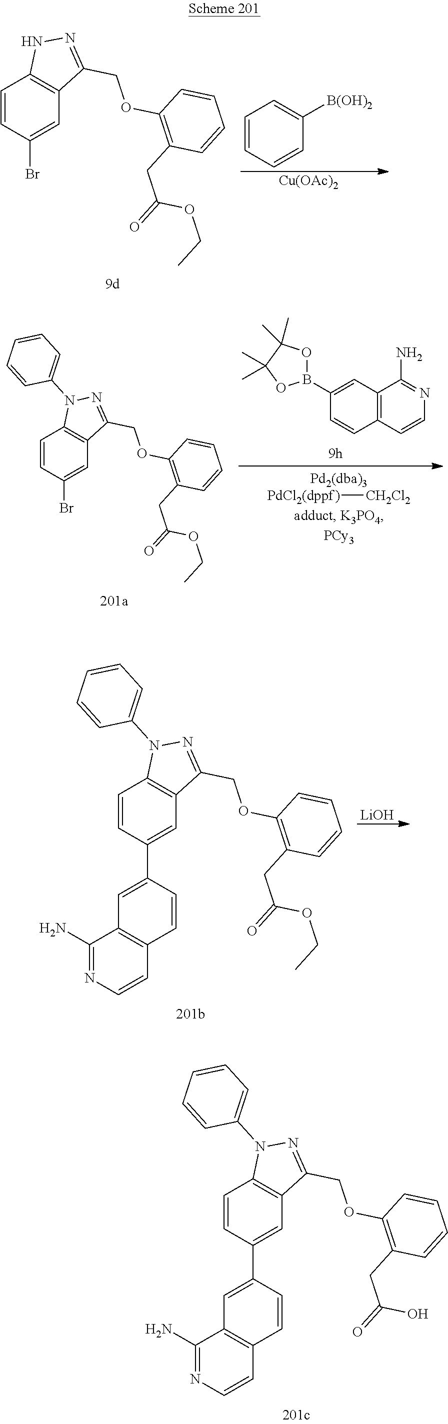

### Preparation of 2-(2-((5-(1-aminoisoquinolin-7-yl)-1-phenyl-1H-indazol-3-yl)methoxy)phenyl)acetic acid (201c)

### Step-1: Preparation of ethyl 2-(2-(5-bromo-1-phenyl-1H-indazol-3-yl)methoxy)phenyl)acetate (201a)

To a solution of ethyl 2-(2-((5-bromo-1H-indazol-3-yl)methoxy)phenyl)acetate (9d) (1 g, 2.57 mmol) in THF (20 mL) was added phenylboronic acid (0.626 g, 5.14 mmol), pyridine (0.208 mL, 2.57 mmol), copper (II) acetate (0.233 g, 1.285 mmol) and 4° A molecular sieves (1 g). The mixture was heated in open air for 15 h at 50° ° C. The reaction mixture was filtered through a pad of celite and the filtrate was concentrated in vacuo. The residue obtained was purified using method-K to give ethyl 2-(2-((5-bromo-1-phenyl-1H-indazol-3-yl)methoxy)phenyl)acetate (201a) (0.85 g, 71.1% yield) as a clear oil; MS (ES+): 465.00 & 467.10 (M+1).

### Step-2: Preparation of ethyl 2-(2-((5-(1-aminoisoquinolin-7-yl)-1-phenyl-1H-indazol-3-yl)methoxy)phenyl)acetate (201b)

Compound 201b was prepared according to the procedure reported in step-5 of scheme 1 from ethyl 2-(2-((5-bromo-1-phenyl-1H-indazol-3-yl)methoxy)phenyl)acetate (201a) (200 mg, 0.430 mmol) in dioxane/THF (4 mL, 1:1) using 7-(4,4,5,5-tetramethyl-1,3,2-dioxaborolan-2-yl)isoquinolin-1-amine (9h) (174 mg, 0.645 mmol), K₃PO₄ (2 M aqueous solution, 0.860 mL, 1.719 mmol), PCy₃ (24.11 mg, 0.086 mmol), PdCl₂(dppf)-CH₂Cl₂ adduct (35.1 mg, 0.043 mmol) and Pd₂(dba)₃ (39.4 mg, 0.043 mmol) to afford after workup and purification using method-Y, ethyl 2-(2-((5-(1-aminoisoquinolin-7-yl)-1-phenyl-1H-indazol-3-yl)methoxy)phenyl)acetate (201b) (120 mg, 52.8% yield) as a clear oil; 1H NMR (300 MHz, DMSO-d₆) δ 8.60 (s, 1H), 8.32 (s, 1H), 8.12-8.04 (m, 2H), 8.00 (d, J=8.8 Hz, 1H), 7.88-7.78 (m, 4H), 7.65 (t, J=7.7 Hz, 2H), 7.46 (t, J=7.3 Hz, 1H), 7.36-7.31 (m, 2H), 7.21 (d, J=7.2 Hz, 1H), 7.01 (s, 2H, D₂O exchangeable), 6.94 (t, J=6.8 Hz, 2H), 5.58 (s, 2H), 3.63 (q, J=7.2 Hz, 2H), 3.57 (s, 2H), 0.72 (dd, J=7.7, 6.5 Hz, 3H); MS (ES+): 529.20 (M+1).

### Step-3: Preparation of 2-(2-((5-(1-aminoisoquinolin-7-yl)-1-phenyl-1H-indazol-3-yl)methoxy)phenyl)acetic acid (201c)

Compound 201c was prepared according to the procedure reported in step-2 of scheme 1 from ethyl 2-(2-((5-(1-aminoisoquinolin-7-yl)-1-phenyl-1H-indazol-3-yl)methoxy)phenyl)acetate (201b) (120 mg, 0.227 mmol) in MeOH/THF (6 mL, 1:1) using a solution of LiOH·H₂O (57.2 mg, 1.362 mmol) in water (2 mL) to afford after workup and purification using method-G, 2-(2-((5-(1-aminoisoquinolin-7-yl)-1-phenyl-1H-indazol-3-yl)methoxy)phenyl)acetic acid (201c) (65 mg, 57.2% yield) HCl salt as a white solid; ¹H NMR (300 MHz, DMSO-d₆) δ 13.31 (s, 1H, D₂O exchangeable), 12.17 (s, 1H, D₂O) exchangeable), 9.17 (s, 2H, D₂O exchangeable), 9.01 (s, 1H), 8.49-8.39 (m, 2H), 8.16-7.98 (m, 3H), 7.90-7.79 (m, 2H), 7.75-7.58 (m, 3H), 7.47 (t, J=7.4 Hz, 1H), 7.37-7.26 (m, 3H), 7.22 (d, J=7.3 Hz, 1H), 6.94 (t, J=7.2 Hz, 1H), 5.61 (s, 2H), 3.57 (s, 2H); MS (ES+): 501.20 (M+1); MS (ES−): 499.10 (M−1).

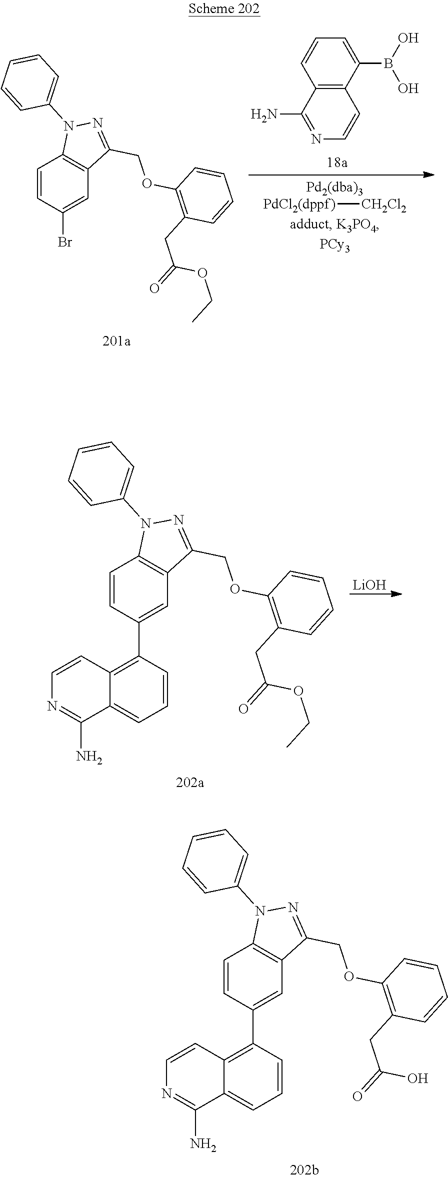

### Preparation of 2-(2-((5-(1-aminoisoquinolin-5-yl)-1-phenyl-1H-indazol-3-yl)methoxy)phenyl)acetic acid (202b)

### Step-1: Preparation of ethyl 2-(2-((5-(1-aminoisoquinolin-5-yl)-1-phenyl-1H-indazol-3-yl)methoxy)phenyl)acetate (202a)

Compound 202a was prepared according to the procedure reported in step-5 of scheme 1 from ethyl 2-(2-((5-bromo-1-phenyl-1H-indazol-3-yl)methoxy)phenyl)acetate (201a) (200 mg, 0.430 mmol) in dioxane/THF (4 mL, 1:1) using (1-aminoisoquinolin-5-yl)boronic acid (18a) (121 mg, 0.645 mmol), K₃PO₄ (2 M aqueous solution, 0.860 mL, 1.719 mmol), PCy₃ (24.11 mg, 0.086 mmol), PdCl₂(dppf)-CH₂Cl₂ adduct (35.1 mg, 0.043 mmol) and Pd₂(dba)₃ (39.4 mg, 0.043 mmol) to afford after workup and purification using method-Y, ethyl 2-(2-((5-(1-aminoisoquinolin-5-yl)-1-phenyl-1H-indazol-3-yl)methoxy)phenyl)acetate (202a) (120 mg, 52.8% yield) as a clear oil; ¹H NMR (300 MHz, DMSO-d₆) δ 8.25 (d, J=8.2 Hz, 1H), 8.02-7.92 (m, 2H), 7.85 (d, J=8.4 Hz, 2H), 7.77 (d, J=6.0 Hz, 1H), 7.69-7.51 (m, 5H), 7.46 (t, J=7.4 Hz, 1H), 7.33-7.24 (m, 2H), 7.18 (d, J=7.3 Hz, 1H), 6.97-6.87 (m, 3H, 2H exchangeable), 6.84 (d, J=6.1 Hz, 1H), 5.55 (s, 2H), 3.63-3.48 (m, 4H), 0.71 (t, J=7.1 Hz, 3H); MS (ES+): 529.25 (M+1).

### Step-2: Preparation of 2-(2-((5-(1-aminoisoquinolin-5-yl)-1-phenyl-1H-indazol-3-yl)methoxy)phenyl)acetic acid (202b)

Compound 202b was prepared according to the procedure reported in step-2 of scheme 1 from ethyl 2-(2-((5-(1-aminoisoquinolin-5-yl)-1-phenyl-1H-indazol-3-yl)methoxy)phenyl)acetate (202a) (120 mg, 0.227 mmol) in MeOH/THF (6 mL, 1:1) using a solution of LiOH·H₂O (57.2 mg, 1.362 mmol) in water (2 mL) to afford after workup and purification using method-G, 2-(2-((5-(1-aminoisoquinolin-5-yl)-1-phenyl-1H-indazol-3-yl)methoxy)phenyl)acetic acid (202b) (60 mg, 52.8% yield) HCl salt as a white solid; ¹H NMR (300 MHz, DMSO-d₆) δ 13.34 (s, 1H, D₂O exchangeable), 12.05 (s, 1H, D₂O exchangeable), 9.15 (s, 2H, D₂O exchangeable), 8.64 (d, J=8.3 Hz, 1H), 8.07-7.94 (m, 3H), 7.91-7.81 (m, 3H), 7.70-7.62 (m, 3H), 7.59 (dd, J=8.8, 1.7 Hz, 1H), 7.47 (t, J=7.4 Hz, 1H), 7.34-7.22 (m, 2H), 7.19 (d, J=7.3 Hz, 1H), 7.00 (d, J=7.3 Hz, 1H), 6.97-6.87 (m, 1H), 5.57 (s, 2H), 3.52 (s, 2H); MS (ES+): 501.15 (M+1); MS (ES−): 499.10 (M−1).

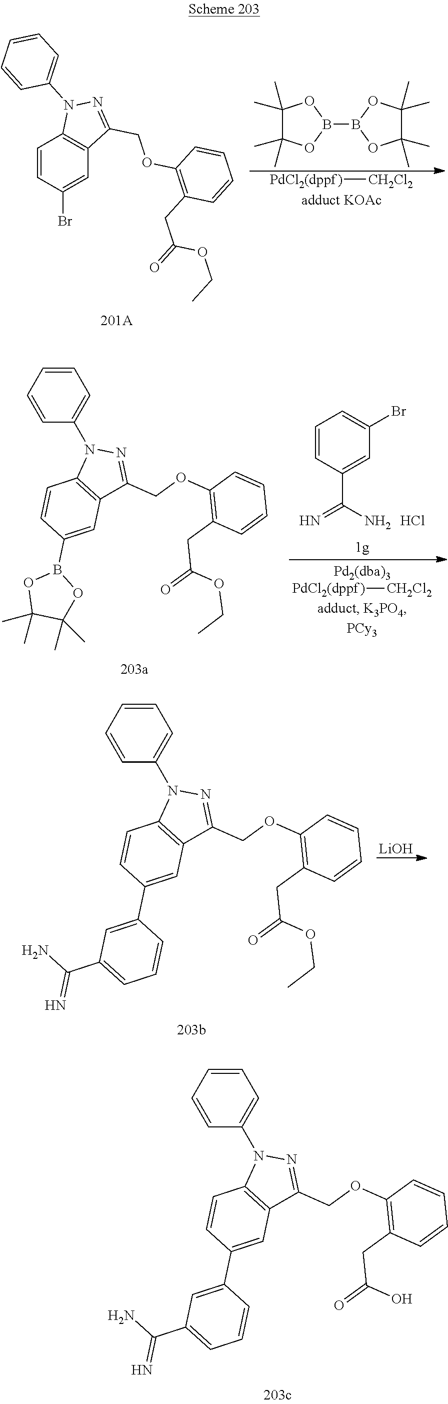

### Preparation of 2-(2-((5-(3-carbamimidoylphenyl)-1-phenyl-1H-indazol-3-yl)methoxy)phenyl)acetic acid (203c)

### Step-1: Preparation of ethyl 2-(2-((1-phenyl-5-(4,4,5,5-tetramethyl-1,3,2-dioxaborolan-2-yl)-1H-indazol-3-yl)methoxy)phenyl)acetate (203a)

Compound 203a was prepared according to the procedure reported in step-4 of scheme 1 from ethyl 2-(2-((5-bromo-1-phenyl-1H-indazol-3-yl)methoxy)phenyl)acetate (201a) (450 mg, 0.967 mmol) in anhydrous dioxane (20 mL) using BISPIN (491 mg, 1.934 mmol), KOAc (237 mg, 2.418 mmol) and PdCl₂(dppf)-CH₂Cl₂ adduct (47.4 mg, 0.058 mmol) to afford after workup and purification using method-AA, ethyl 2-(2-((1-phenyl-5-(4,4,5,5-tetramethyl-1,3,2-dioxaborolan-2-yl)-1H-indazol-3-yl)methoxy)phenyl)acetate (203a) (400 mg, 81% yield) as a clear oil; 1H NMR (300 MHz, DMSO-d₆) δ 8.29 (s, 1H), 7.85 (d, J=8.5 Hz, 1H), 7.81-7.73 (m, 3H), 7.63 (t, J=7.8 Hz, 2H), 7.44 (t, J=7.3 Hz, 1H), 7.32 (d, J=4.0 Hz, 2H), 7.22 (d, J=7.4 Hz, 1H), 6.99-6.90 (m, 1H), 5.53 (s, 2H), 3.86 (q, J=7.2 Hz, 2H), 3.55 (s, 2H), 1.32 (s, 12H), 0.87 (t, J=7.1 Hz, 3H); MS (ES+): 513.20 (M+1).

### Step-2: Preparation of ethyl 2-(2-((5-(3-carbamimidoylphenyl)-1-phenyl-1H-indazol-3-yl)methoxy)phenyl)acetate (203b)

Compound 203b was prepared according to the procedure reported in step-5 of scheme 1 from ethyl 2-(2-((1-phenyl-5-(4,4,5,5-tetramethyl-1,3,2-dioxaborolan-2-yl)-1H-indazol-3-yl)methoxy)phenyl)acetate (203a) (400 mg, 0.781 mmol) in dioxane/THF (4 mL, 1:1) using 3-bromobenzimidamide hydrochloride (1g) (368 mg, 1.561 mmol), K₃PO₄ (2 M aqueous solution, 1.561 mL, 3.12 mmol), PCy₃ (43.8 mg, 0.156 mmol), PdCl₂(dppf)-CH₂Cl₂ adduct (63.7 mg, 0.078 mmol) and Pd₂(dba)₃ (71.5 mg, 0.078 mmol) to afford after workup and purification using method-AK, ethyl 2-(2-((5-(3-carbamimidoylphenyl)-1-phenyl-1H-indazol-3-yl)methoxy)phenyl)acetate (203b) (200 mg, 50.8% yield) as a clear oil; 1H NMR (300 MHz, DMSO-d₆) δ 9.09 (s, 4H), 8.30 (s, 1H), 8.18-8.07 (m, 2H), 8.01-7.96 (m, 1H), 7.86-7.70 (m, 4H), 7.68-7.60 (m, 3H), 7.46 (t, J=7.5 Hz, 1H), 7.36-7.26 (m, 2H), 7.21 (d, J=7.3 Hz, 1H), 6.98-6.89 (m, 1H), 5.57 (s, 2H), 3.67 (q, J=7.1 Hz, 2H), 3.59-3.54 (m, 2H), 0.78 (t, J=7.1 Hz, 3H); MS (ES+): 505.20 (M+1).

### Step-3: Preparation of 2-(2-((5-(3-carbamimidoylphenyl)-1-phenyl-1H-indazol-3-yl)methoxy)phenyl)acetic acid (203c)

Compound 203c was prepared according to the procedure reported in step-2 of scheme 1 from ethyl 2-(2-((5-(3-carbamimidoylphenyl)-1-phenyl-1H-indazol-3-yl)methoxy)phenyl)acetate (203b) (200 mg, 0.396 mmol) in MeOH/THF (6 mL, 1:1) using a solution of LiOH·H₂O (100 mg, 2.378 mmol) in water (2 mL) to afford after workup and purification using method-G, 2-(2-((5-(3-carbamimidoylphenyl)-1-phenyl-1H-indazol-3-yl)methoxy)phenyl)acetic acid (203c) (45 mg, 23.82% yield) HCl salt as a white solid; ¹H NMR (300 MHz, DMSO-d₆) δ 12.13 (s, 1H, D₂O exchangeable), 9.51 (s, 2H, D₂O exchangeable), 9.25 (s, 2H, D₂O exchangeable), 8.37 (s, 1H), 8.21 (s, 1H), 8.14 (d, J=7.5 Hz, 1H), 8.00 (s, 2H), 7.89-7.79 (m, 3H), 7.73 (t, J=7.7 Hz, 1H), 7.64 (t, J=7.7 Hz, 2H), 7.46 (t, J=7.4 Hz, 1H), 7.38-7.26 (m, 2H), 7.22 (d, J=7.3 Hz, 1H), 6.94 (t, J=7.3 Hz, 1H), 5.59 (s, 2H), 3.57 (s, 2H); MS (ES+): 477.20 (M+1); (ES−): 475.10 (M−1).

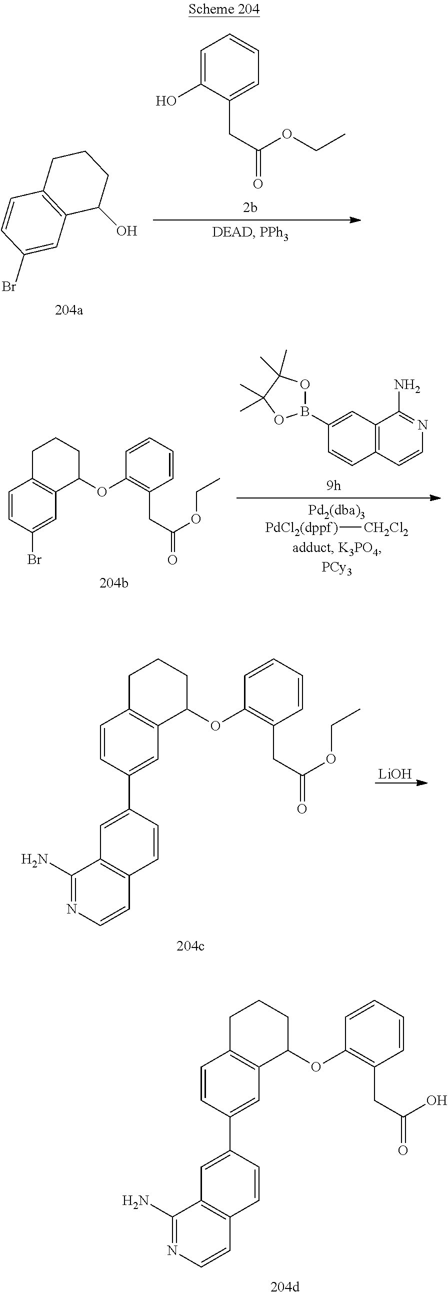

### Preparation of 2-(2-((7-(1-aminoisoquinolin-7-yl)-1,2,3,4-tetrahydronaphthalen-1-yl)oxy)phenyl)acetic acid (204d)

### Step-1: Preparation of ethyl 2-(2-((7-bromo-1,2,3,4-tetrahydronaphthalen-1-yl)oxy)phenyl)acetate (204b)

Compound 204b was prepared according to the procedure reported in step-1 of scheme 8 from 7-bromo-1,2,3,4-tetrahydronaphthalen-1-ol (204a) (500 mg, 2.202 mmol; CAS #75693-15-1) in DCM (10 mL) using ethyl 2-(2-hydroxyphenyl)acetate (2b) (436 mg, 2.422 mmol), PPh₃ (693 mg, 2.64 mmol) and a solution of DEAD (460 mg, 2.64 mmol) in DCM (10 mL) to afford after workup and purification using method-P, ethyl 2-(2-((7-bromo-1,2,3,4-tetrahydronaphthalen-1-yl)oxy)phenyl)acetate (204b) (160 mg, 18.67% yield) as a white oil; ¹H NMR (300 MHz, DMSO-d₆) δ 7.44-7.36 (m, 2H), 7.33-7.25 (m, 1H), 7.25-7.19 (m, 2H), 7.14 (d, J=8.0 Hz, 1H), 6.95-6.88 (m, 1H), 5.53 (t, J=5.0 Hz, 1H), 3.94 (g, J=7.1 Hz, 2H), 3.48 (s, 2H), 2.81-2.65 (m, 2H), 1.99-1.73 (m, 4H), 1.06-0.97 (m, 3H); MS (ES+): 411.00 & 413.00 (M+Na); MS (ES−): 387.10 & 389.00 (M−1).

### Step-2: Preparation of ethyl 2-(2-((7-(1-aminoisoquinolin-7-yl)-1,2,3,4-tetrahydronaphthalen-1-yl)oxy)phenyl)acetate (204c)

Compound 204c was prepared according to the procedure reported in step-5 of scheme 1 from ethyl 2-(2-((7-bromo-1,2,3,4-tetrahydronaphthalen-1-yl)oxy)phenyl)acetate (204b) (160 mg, 0.411 mmol) in dioxane/THF (4 mL, 1:1) using 7-(4,4,5,5-tetramethyl-1,3,2-dioxaborolan-2-yl)isoquinolin-1-amine (9h) (167 mg, 0.617 mmol), K₃PO₄ (2 M aqueous solution, 0.822 mL, 1.644 mmol), PCy₃ (23.05 mg, 0.082 mmol), PdCl₂(dppf)-CH₂Cl₂ adduct (33.6 mg, 0.041 mmol) and Pd₂(dba)₃ (37.6 mg, 0.041 mmol) to afford after workup and purification using method-Y, ethyl 2-(2-((7-(1-aminoisoquinolin-7-yl)-1,2,3,4-tetrahydronaphthalen-1-yl)oxy)phenyl)acetate (204c) (140 mg, 75% yield) as a clear oil; ¹H NMR (300 MHz, DMSO-d₆) δ 8.46 (s, 1H), 7.89 (d, J=8.5 Hz, 1H), 7.79-7.71 (m, 3H), 7.69 (s, 1H), 7.34-7.27 (m, 3H), 7.21 (d, J=7.4 Hz, 1H), 6.97-6.86 (m, 4H, 2H exchangeable), 5.61 (m, 1H), 3.82-3.57 (m, 2H), 3.47 (s, 2H), 2.96-2.70 (m, 2H), 2.06-1.97 (m, 2H), 1.68-1.63 (m, 2H), 0.84 (t, J=7.1 Hz, 3H); MS (ES+): 453.20 (M+1).

### Step-3: Preparation of 2-(2-((7-(1-aminoisoquinolin-7-yl)-1,2,3,4-tetrahydronaphthalen-1-yl)oxy)phenyl)acetic acid (204d)

Compound 204d was prepared according to the procedure reported step-2 of scheme 1 from ethyl 2-(2-((7-(1-aminoisoquinolin-7-yl)-1,2,3,4-tetrahydronaphthalen-1-yl)oxy)phenyl)acetate (204c) (140 mg, 0.309 mmol) in MeOH/THF (6 mL, 1:1) using a solution of LiOH·H₂O (78 mg, 1.856 mmol) in water (2 mL) to afford after workup and purification using method-G, 2-(2-((7-(1-aminoisoquinolin-7-yl)-1,2,3,4-tetrahydronaphthalen-1-yl)oxy)phenyl)acetic acid (204d) (55 mg, 41.9% yield) HCl salt as a white solid; ¹H NMR (300 MHz, DMSO-d₆) δ 13.98-11.45 (m, 2H, D₂O exchangeable), 9.17 (s, 2H, D₂O exchangeable), 8.89 (s, 1H), 8.26 (d, J=8.5 Hz, 1H), 8.05-7.96 (m, 1H), 7.86-7.76 (m, 2H), 7.73-7.63 (m, 1H), 7.40-7.27 (m, 3H), 7.27-7.18 (m, 2H), 6.97-6.84 (m, 1H), 5.67-5.55 (m, 1H), 3.46 (s, 2H), 3.00-2.71 (m, 2H), 2.11-1.70 (m, 4H); MS (ES+): 425.20 (M+1); (ES−): 423.10 (M−1).

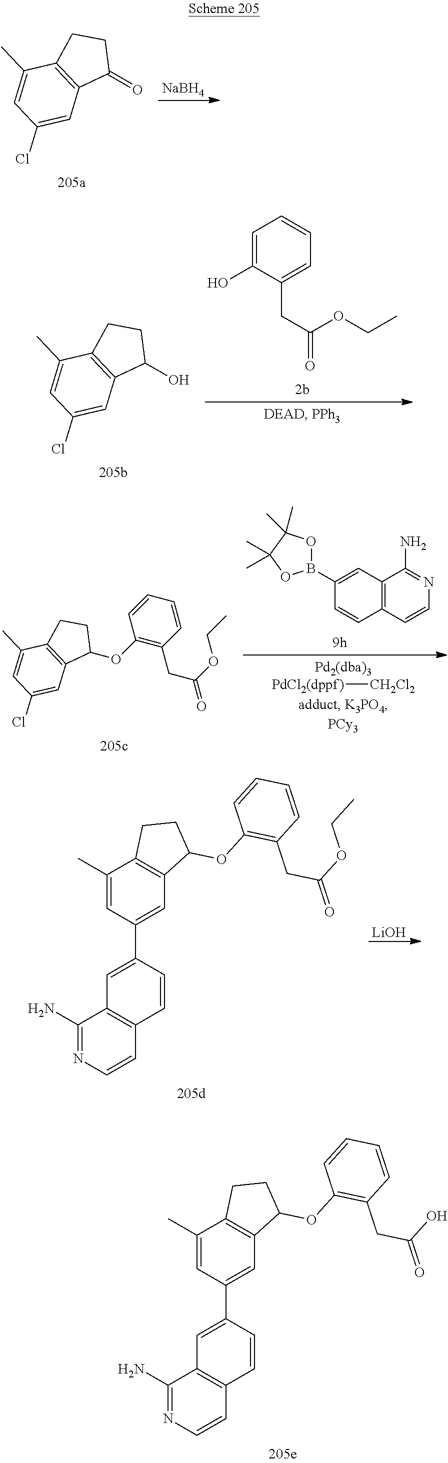

### Preparation of 2-(2-((6-(1-aminoisoquinolin-7-yl)-4-methyl-2,3-dihydro-1H-inden-1-yl)oxy)phenyl)acetic acid (205e)

### Step-1: Preparation of 6-chloro-4-methyl-2,3-dihydro-1H-inden-1-ol (205b)

To a stirred solution of 6-chloro-4-methyl-2,3-dihydro-1H-inden-1-one (205a) (900 mg, 4.98 mmol; CAS #938-35-2) in anhydrous MeOH (30 mL) was added NaBH₄ (283 mg, 7.47 mmol) at 0° C., in small portions over a period of 10 min and stirred for 1 h. The reaction mixture was treated with brine (100 mL) and extracted with ethyl acetate (2×150 mL). Organic layer were combined, dried, filtered and concentrated in vacuum to give 6-chloro-4-methyl-2,3-dihydro-1H-inden-1-ol (205b) (830 mg, 91% yield) as a white oil; 1H NMR (300 MHz, DMSO-d₆) δ 7.13 (s, 1H), 7.09 (s, 1H), 5.32 (d, J=5.8 Hz, 1H), 5.02 (q, J=6.6 Hz, 1H), 2.89-2.73 (m, 1H), 2.66-2.53 (m, 1H), 2.42-2.27 (m, 1H), 2.20 (s, 3H), 1.86-1.67 (m, 1H).

### Step-2: Preparation of ethyl 2-(2-((6-chloro-4-methyl-2,3-dihydro-1H-inden-1-yl)oxy)phenyl)acetate (205c)

Compound 205c was prepared according to the procedure reported in step-1 of scheme 8 from 6-chloro-4-methyl-2,3-dihydro-1H-inden-1-ol (205b) (830 mg, 4.54 mmol) in THF (30 mL) using ethyl 2-(2-hydroxyphenyl)acetate (2b) (1638 mg, 9.09 mmol), PPh₃ (2384 mg, 9.09 mmol) and a solution of DEAD (1583 mg, 9.09 mmol) in THF to afford after workup and purification using method-P, ethyl 2-(2-((6-chloro-4-methyl-2,3-dihydro-1H-inden-1-yl)oxy)phenyl)acetate (205c) (600 mg, 38.3% yield) as a white oil; 1H NMR (300 MHZ, DMSO-d₆) ¿ 7.29 (t, J=7.8 Hz, 1H), 7.24-7.11 (m, 4H), 6.96-6.86 (m, 1H), 5.81 (t, J=5.7 Hz, 1H), 4.01-3.86 (m, 2H), 3.49 (s, 2H), 2.98-2.85 (m, 1H), 2.84-2.70 (m, 1H), 2.70-2.57 (m, 1H), 2.25 (s, 3H), 2.03-1.89 (m, 1H), 1.04 (t, J=7.0 Hz, 3H); MS (ES+): 345.10 (M+1); 367.10 & 369.10 (M+Na).

### Step-3: Preparation of ethyl 2-(2-((6-(1-aminoisoquinolin-7-yl)-4-methyl-2,3-dihydro-1H-inden-1-yl)oxy)phenyl)acetate (205d)

Compound 205d was prepared according to the procedure reported in step-5 of scheme 1 from ethyl 2-(2-((6-chloro-4-methyl-2,3-dihydro-1H-inden-1-yl)oxy)phenyl)acetate (205c) (200 mg, 0.58 mmol) in dioxane/THF (4 mL, 1:1) using 7-(4,4,5,5-tetramethyl-1,3,2-dioxaborolan-2-yl)isoquinolin-1-amine (9h) (235 mg, 0.870 mmol), K₃PO₄ (2 M aqueous solution, 1.160 mL, 2.32 mmol), PCy₃ (32.5 mg, 0.116 mmol), PdCl₂(dppf)-CH₂Cl₂ adduct (47.4 mg, 0.058 mmol) and Pd₂(dba)₃ (53.1 mg, 0.058 mmol) to afford after workup and purification using method-Y, ethyl 2-(2-((6-(1-aminoisoquinolin-7-yl)-4-methyl-2,3-dihydro-1H-inden-1-yl)oxy)phenyl)acetate (205d) (200 mg, 76% yield) as a clear oil; 1H NMR (300 MHz, DMSO-d₆) δ 8.50 (s, 1H), 7.93 (d, J=9.4 Hz, 1H), 7.77 (d, J=7.0 Hz, 2H), 7.63 (d, J=15.2 Hz, 2H), 7.31 (t, J=7.8 Hz, 1H), 7.27-7.17 (m, 2H), 7.02 (s, 2H), 6.93 (d, J=6.6 Hz, 2H), 5.89 (d, J=6.0 Hz, 1H), 3.78 (q. J=7.0 Hz, 2H), 3.49 (s, 2H), 3.07-2.92 (m, 1H), 2.92-2.78 (m, 1H), 2.75-2.61 (m, 1H), 2.37 (s, 3H), 2.07-1.91 (m, 1H), 0.79 (t, J=7.1 Hz, 3H); MS (ES+): 453.20 (M+1);

### Step-4: Preparation of 2-(2-((6-(1-aminoisoquinolin-7-yl)-4-methyl-2,3-dihydro-1H-inden-1-yl)oxy)phenyl)acetic acid (205e)

Compound 205e was prepared according to the procedure reported step-2 of scheme 1 from ethyl 2-(2-((6-(1-aminoisoquinolin-7-yl)-4-methyl-2,3-dihydro-1H-inden-1-yl)oxy)phenyl)acetate (205d) (200 mg, 0.442 mmol) in MeOH/THF (6 mL, 1:1) using a solution of LiOH·H₂O (111 mg, 2.65 mmol) in water (2 mL) to afford after workup and purification using method-G, 2-(2-((6-(1-aminoisoquinolin-7-yl)-4-methyl-2,3-dihydro-1H-inden-1-yl)oxy)phenyl)acetic acid (205e) (50 mg, 26.7% yield) HCl salt as a white solid; 1H NMR (300 MHz, DMSO-d₆) 13.29 (s, 1H, D₂O exchangeable), 12.11 (s, 1H, D₂O exchangeable), 9.16 (s, 2H, D₂O exchangeable), 8.88 (s, 1H), 8.30 (d, J=8.5 Hz, 1H), 8.03 (d, J=8.4 Hz, 1H), 7.75-7.63 (m, 3H), 7.33-7.16 (m, 4H), 6.92 (t, J=7.2 Hz, 1H), 5.95-5.82 (m, 1H), 3.47 (s, 2H), 3.11-2.96 (m, 1H), 2.93-2.79 (m, 1H), 2.76-2.63 (m, 1H), 2.38 (s, 3H), 2.10-1.95 (m, 1H); MS (ES+): 425.15 (M+1); MS (ES−): 423.10 (M−1).

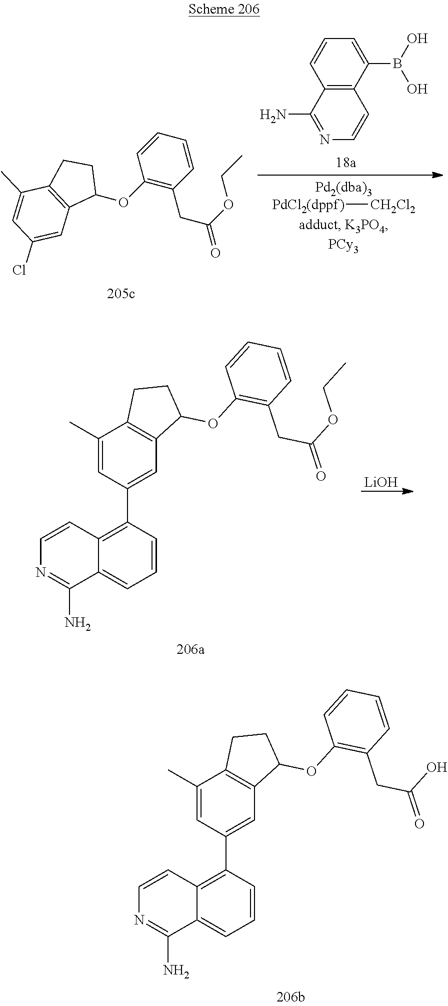

### Preparation of 2-(2-((6-(1-aminoisoquinolin-5-yl)-4-methyl-2,3-dihydro-1H-inden-1-yl)oxy)phenyl)acetic acid (206b)

### Step-1: Preparation of ethyl 2-(2-((6-(1-aminoisoquinolin-5-yl)-4-methyl-2,3-dihydro-1H-inden-1-yl)oxy)phenyl)acetate (206a)

Compound 206a was prepared according to the procedure reported in step-5 of scheme 1 from ethyl 2-(2-((6-chloro-4-methyl-2,3-dihydro-1H-inden-1-yl)oxy)phenyl)acetate acetate (205c) (200 mg, 0.58 mmol) in dioxane/THF (4 mL, 1:1) using (1-aminoisoquinolin-5-yl)boronic acid (18a) (164 mg, 0.87 mmol), K₃PO₄ (2 M aqueous solution, 1.160 mL, 2.32 mmol), PCy₃ (32.5 mg, 0.116 mmol), PdCl₂(dppf)-CH₂Cl₂ adduct (47.4 mg, 0.058 mmol) and Pd₂(dba)₃ (53.1 mg, 0.058 mmol) to afford after workup and purification using method-Y, ethyl 2-(2-((6-(1-aminoisoquinolin-5-yl)-4-methyl-2,3-dihydro-1H-inden-1-yl)oxy)phenyl)acetate acetate (206a) (100 mg, 38.1% yield) as a clear oil; MS (ES+): 453.20 (M+1).

### Step-2: Preparation of 2-(2-((6-(1-aminoisoquinolin-5-yl)-4-methyl-2,3-dihydro-1H-inden-1-yl)oxy)phenyl)acetic acid (206b)

Compound 206b was prepared according to the procedure reported step-2 of scheme 1 from ethyl 2-(2-((6-(1-aminoisoquinolin-5-yl)-4-methyl-2,3-dihydro-1H-inden-1-yl)oxy)phenyl)acetate acetate (206a) (100 mg, 0.221 mmol) in MeOH/THF (6 mL, 1:1) using LiOH·H₂O (55.6 mg, 1.326 mmol) in water (2 mL) to afford after workup and purification using method-G, 2-(2-((6-(1-aminoisoquinolin-5-yl)-4-methyl-2,3-dihydro-1H-inden-1-yl)oxy)phenyl)acetic acid (206b) (26 mg, 27.7% yield) HCl salt as a white solid; 1H NMR (300 MHz, DMSO-d₆) δ 13.17 (s, 1H, D₂O) exchangeable), 12.01 (s, 1H, D₂O exchangeable), 9.11 (s, 2H, D₂O exchangeable), 8.58 (d, J=8.0 Hz, 1H), 7.93-7.76 (m, 2H), 7.60 (d, J=7.2 Hz, 1H), 7.30-7.13 (m, 5H), 6.97 (d, J=7.2 Hz, 1H), 6.90 (t, J=7.2 Hz, 1H), 5.94-5.83 (m, 1H), 3.43 (s, 2H), 3.13-3.00 (m, 1H), 2.96-2.82 (m, 1H), 2.81-2.66 (m, 1H), 2.35 (s, 3H), 2.12-1.97 (m, 1H); MS (ES+): 425.20 (M+1); (ES−): 423.10 (M−1).

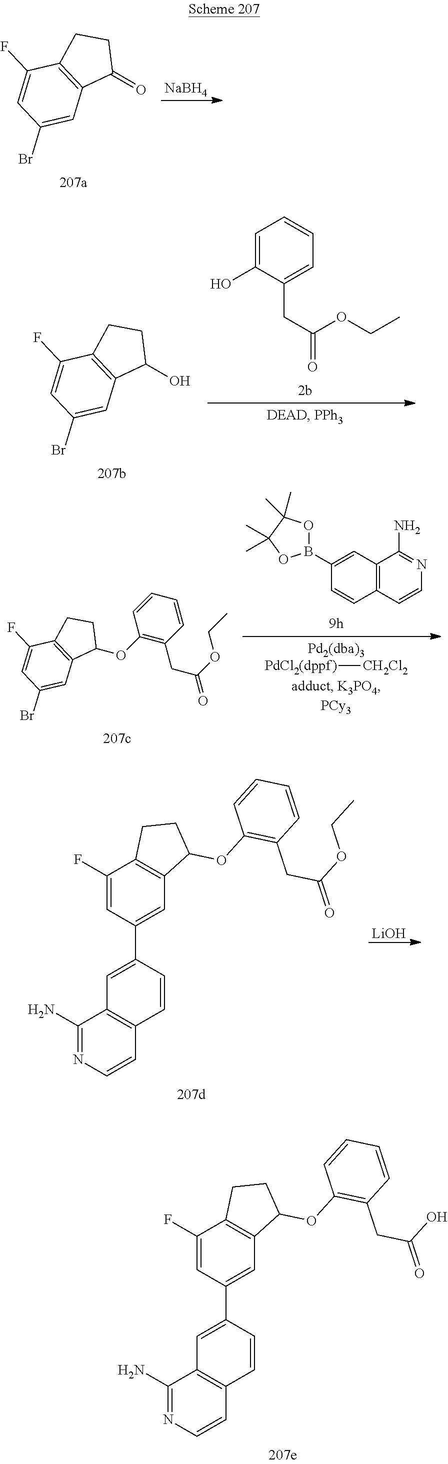

### Preparation of 2-(2-((6-(1-aminoisoquinolin-7-yl)-4-fluoro-2,3-dihydro-1H-inden-1-yl)oxy)phenyl)acetic acid (207e)

### Step-1: Preparation of 6-bromo-4-fluoro-2,3-dihydro-1H-inden-1-ol (207b)

Compound 207b was prepared according to the procedure reported in step-1 of scheme 205 from 6-bromo-4-fluoro-2,3-dihydro-1H-inden-1-one (207a) (950 mg, 4.15 mmol) in anhydrous MeOH (20 mL) using NaBH₄ (235 mg, 6.22 mmol) to afford after workup, 6-bromo-4-fluoro-2,3-dihydro-1H-inden-1-ol (207b) (840 mg, 88% yield) as a white oil; ¹H NMR (300 MHz, DMSO-d₆) δ 7.34 (d, J=8.2 Hz, 2H), 5.50 (dd, J=5.9, 1.6 Hz, 1H), 5.07 (q, J=6.5 Hz, 1H), 2.98-2.79 (m, 1H), 2.75-2.59 (m, 1H), 2.43-2.29 (m, 1H), 1.91-1.70 (m, 1H).

### Step-2: Preparation of ethyl 2-(2-((6-bromo-4-fluoro-2,3-dihydro-1H-inden-1-yl)oxy)phenyl)acetate (207c)

Compound 207c was prepared according to the procedure reported in step-1 of scheme 8 from 6-bromo-4-fluoro-2,3-dihydro-1H-inden-1-ol (207b) (840 mg, 3.64 mmol) in THF (20 mL) using ethyl 2-(2-hydroxyphenyl)acetate (2b) (1310 mg, 7.27 mmol), PPh₃ (1907 mg, 7.27 mmol) and a solution of DEAD (1266 mg, 7.27 mmol) in THF to afford after workup and purification using method-P, ethyl 2-(2-((6-bromo-4-fluoro-2,3-dihydro-1H-inden-1-yl)oxy)phenyl)acetate (207c) (800 mg, 56.0% yield) as a white oil; ¹H NMR (300 MHZ, DMSO-d₆) δ 7.49 (d, J=8.8 Hz, 1H), 7.36-7.25 (m, 2H), 7.20 (t, J=8.6 Hz, 2H), 6.93 (t, J=7.4 Hz, 1H), 5.88 (t, J=5.8 Hz, 1H), 3.96 (q, J=7.1 Hz, 2H), 3.50 (s, 2H), 3.06-2.94 (m, 1H), 2.94-2.78 (m, 1H), 2.73-2.59 (m, 1H), 2.07-1.93 (m, 1H), 1.05 (t, J=7.1 Hz, 3H); MS (ES+): 393.00 & 395.00 (M+1).

### Step-3: Preparation of ethyl 2-(2-((6-(1-aminoisoquinolin-7-yl)-4-fluoro-2,3-dihydro-1H-inden-1-yl)oxy)phenyl)acetate (207d)

Compound 207d was prepared according to the procedure reported in step-5 of scheme 1 from ethyl 2-(2-((6-bromo-4-fluoro-2,3-dihydro-1H-inden-1-yl)oxy)phenyl)acetate (207c) (200 mg, 0.509 mmol) in dioxane/THF (4 mL, 1:1) using 7-(4,4,5,5-tetramethyl-1,3,2-dioxaborolan-2-yl)isoquinolin-1-amine (9h) (206 mg, 0.763 mmol). K₃PO₄ (2 M aqueous solution, 1.017 mL, 2.034 mmol), PCy₃ (28.5 mg, 0.102 mmol), PdCl₂(dppf)-CH₂Cl₂ adduct (41.5 mg, 0.051 mmol) and Pd₂(dba)₃ (46.6 mg, 0.051 mmol) to afford after workup and purification using method-Y, ethyl 2-(2-((6-(1-aminoisoquinolin-7-yl)-4-fluoro-2,3-dihydro-1H-inden-1-yl)oxy)phenyl)acetate (207d) (100 mg, 43.1% yield) as a clear oil; MS (ES+): 457.20 (M+1).

### Step-4: Preparation of 2-(2-((6-(1-aminoisoquinolin-7-yl)-4-fluoro-2,3-dihydro-1H-inden-1-yl)oxy)phenyl)acetic acid (207e)

Compound 207e was prepared according to the procedure reported step-2 of scheme 1 from ethyl 2-(2-((6-(1-aminoisoquinolin-7-yl)-4-fluoro-2,3-dihydro-1H-inden-1-yl)oxy)phenyl)acetate (207d) (100 mg, 0.219 mmol) in MeOH/THF (6 mL, 1:1) using a solution of LiOH·H₂O (55.2 mg, 1.314 mmol) in water (2 mL) to afford after workup and purification using method-G, 2-(2-((6-(1-aminoisoquinolin-7-yl)-4-fluoro-2,3-dihydro-1H-inden-1-yl)oxy)phenyl)acetic acid (207e) (65 mg, 69.3% yield) HCl salt as a white solid; 1H NMR (300 MHz, DMSO-d₆) δ 13.41 (s, 1H, D₂O exchangeable), 12.16 (s, 1H, D₂O) exchangeable), 9.18 (s, 2H, D₂O exchangeable), 8.95 (s, 1H), 8.35 (d, J=8.5 Hz, 1H), 8.04 (d, J=8.5 Hz, 1H), 7.78 (m, 2H), 7.71 (d, J=6.9 Hz, 1H), 7.36-7.17 (m, 4H), 6.94 (t, J=7.2 Hz, 1H), 5.95 (t, J=5.7 Hz, 1H), 3.48 (s, 2H), 3.20-3.06 (m, 1H), 3.03-2.90 (m, 1H), 2.81-2.65 (m, 1H), 2.15-2.00 (m, 1H); ¹⁹F NMR (282 MHz, DMSO-d₆) δ −117.55; MS (ES+): 429.10 (M+1); (ES−): 427.05 (M−1).

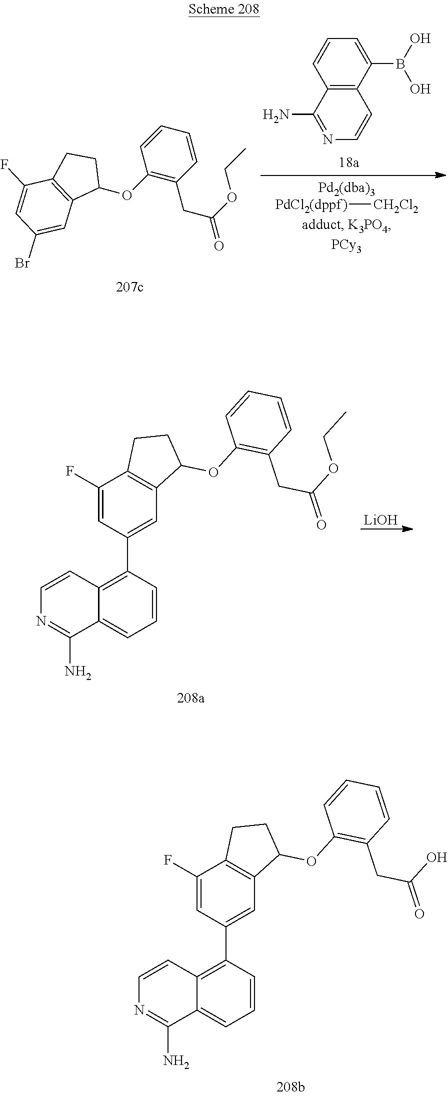

### Preparation of 2-(2-((6-(1-aminoisoquinolin-5-yl)-4-fluoro-2,3-dihydro-1H-inden-1-yl)oxy)phenyl)acetic acid (208b)

### Step-1: Preparation of ethyl 2-(2-((6-(1-aminoisoquinolin-5-yl)-4-fluoro-2,3-dihydro-1H-inden-1-yl)oxy)phenyl)acetate (208a)

Compound 208a was prepared according to the procedure reported in step-5 of scheme 1 from ethyl 2-(2-((6-bromo-4-fluoro-2,3-dihydro-1H-inden-1-yl)oxy)phenyl)acetate (207c) (200 mg, 0.509 mmol) in dioxane/THF (4 mL, 1:1) using (1-aminoisoquinolin-5-yl)boronic acid (18a) (143 mg, 0.763 mmol), K₃PO₄ (2 M aqueous solution, 1.017 mL, 2.034 mmol), PCy₃ (28.5 mg, 0.102 mmol), PdCl₂(dppf)-CH₂Cl₂ adduct (41.5 mg, 0.051 mmol) and Pd₂(dba)₃ (46.6 mg, 0.051 mmol) to afford after workup and purification using method-Y, ethyl 2-(2-((6-(1-aminoisoquinolin-5-yl)-4-fluoro-2,3-dihydro-1H-inden-1-yl)oxy)phenyl)acetate (208a) (100 mg, 43.1% yield) as a clear oil; MS (ES+): 457.20 (M+1).

### Step-2: Preparation of 2-(2-((6-(1-aminoisoquinolin-5-yl)-4-fluoro-2,3-dihydro-1H-inden-1-yl)oxy)phenyl)acetic acid (208b)

Compound 208b was prepared according to the procedure reported step-2 of scheme 1 from ethyl 2-(2-((6-(1-aminoisoquinolin-5-yl)-4-fluoro-2,3-dihydro-1H-inden-1-yl)oxy)phenyl)acetate (208a) (100 mg, 0.219 mmol) in MeOH/THF (6 mL, 1:1) using a solution of LiOH·H₂O (55.2 mg, 1.314 mmol) in water (2 mL) to afford after workup and purification using method-G, 2-(2-((6-(1-aminoisoquinolin-5-yl)-4-fluoro-2,3-dihydro-1H-inden-1-yl)oxy)phenyl)acetic acid (208b) (80 mg, 85% yield) HCl salt as a white solid; 1H NMR (300 MHz, DMSO-d₆) δ 13.39 (s, 1H, D₂O exchangeable), 12.03 (s, 1H, D₂O exchangeable), 9.24 (s, 2H, D₂O exchangeable), 8.64 (d, J=8.2 Hz, 1H), 7.92 (d, J=7.3 Hz, 1H), 7.85 (t, J=7.8 Hz, 1H), 7.62 (d, J=7.2 Hz, 1H), 7.32-7.22 (m, 4H), 7.20 (d, J=7.1 Hz, 1H), 7.00-6.85 (m, 2H), 5.94 (t, J=5.9 Hz, 1H), 3.44 (d, J=5.4 Hz, 2H), 3.23-3.10 (m, 1H), 3.07-2.93 (m, 1H), 2.85-2.69 (m, 1H), 2.19-2.02 (m, 1H); ¹⁹F NMR (282 MHZ, DMSO-d₆) δ −117.57; MS (ES+): 429.10 (M+1); (ES−): 427.00 (M−1).

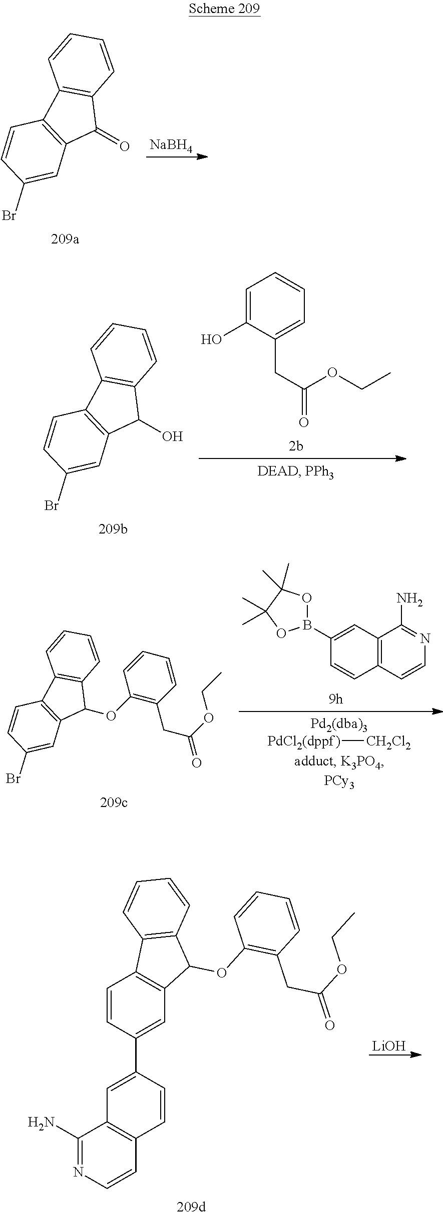

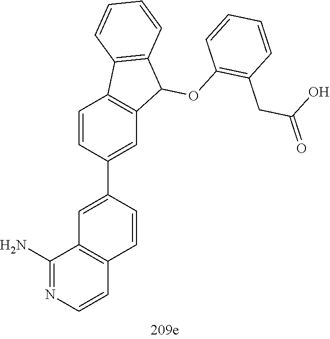 [SMILES: Nc1nccc2ccc(-c3ccc4c(c3)C(Oc3ccccc3CC(=O)O)c3ccccc3-4)cc12]

### Preparation of 2-(2-((2-(1-aminoisoquinolin-7-yl)-9H-fluoren-9-yl)oxy)phenyl)acetic acid (209e)

### Step-1: Preparation of 2-bromo-9H-fluoren-9-ol (209b)

Compound 209b was prepared according to the procedure reported in step-1 of scheme 205 from 2-bromo-9H-fluoren-9-one (209a) (3 g, 11.58 mmol) in anhydrous MeOH (50 mL) using NaBH₄ (0.657 g, 17.37 mmol) to afford after workup, 2-bromo-9H-fluoren-9-ol (209b) (2.7 g, 89% yield) as a yellow solid; ¹H NMR (300 MHz, DMSO-d₆) δ 7.87-7.68 (m, 3H), 7.64-7.52 (m, 2H), 7.46-7.29 (m, 2H), 6.06-5.78 (m, 1H), 5.58-5.40 (m, 1H).

### Step-2: Preparation of ethyl 2-(2-((2-bromo-9H-fluoren-9-yl)oxy)phenyl)acetate (209c)

Compound 209c was prepared according to the procedure reported in step-1 of scheme 8 from 2-bromo-9H-fluoren-9-ol (209b) (1000 mg, 3.83 mmol) in THF (30 mL) using ethyl 2-(2-hydroxyphenyl)acetate (2b) (1380 mg, 7.66 mmol), PPh₃ (2009 mg, 7.66 mmol) and a solution of DEAD (1334 mg, 7.66 mmol) in THE to afford after workup and purification using method-AO, ethyl 2-(2-((2-bromo-9H-fluoren-9-yl)oxy)phenyl)acetate (209c) (200 mg, 12.34% yield) as a white oil; ¹H NMR (300 MHz, DMSO-d₆) δ 7.93-7.83 (m, 2H), 7.70-7.62 (m, 1H), 7.57 (s, 1H), 7.51-7.40 (m, 3H), 7.34 (t, J=7.6 Hz, 2H), 7.27 (d, J=7.2 Hz, 1H), 6.99 (t, J=7.3 Hz, 1H), 6.61 (s, 1H), 3.91 (q, J=7.1 Hz, 2H), 3.50 (s, 2H), 0.96 (t, J=7.1 Hz, 3H); MS (ES+): 423.00 & 425.00 (M+1); 445.00 & 447.00 (M+Na); MS (ES−): 421.00 & 423.00 (M−1).

### Step-3: Preparation of ethyl 2-(2-((2-(1-aminoisoquinolin-7-yl)-9H-fluoren-9-yl)oxy)phenyl)acetate (209d)

Compound 209d was prepared according to the procedure reported in step-5 of scheme 1 from ethyl 2-(2-((2-bromo-9H-fluoren-9-yl)oxy)phenyl)acetate (209c) (100 mg, 0.236 mmol) in dioxane/THF (4 mL, 1:1) using 7-(4,4,5,5-tetramethyl-1,3,2-dioxaborolan-2-yl)isoquinolin-1-amine (9h) (96 mg, 0.354 mmol), K₃PO₄ (2 M aqueous solution, 0.472 mL, 0.945 mmol), PCy₃ (13.25 mg, 0.047 mmol), PdCl₂(dppf)-CH₂Cl₂ adduct (19.29 mg, 0.024 mmol) and Pd₂(dba)₃ (21.63 mg, 0.024 mmol) to afford after workup and purification using method-Y, ethyl 2-(2-((2-(1-aminoisoquinolin-7-yl)-9H-fluoren-9-yl)oxy)phenyl)acetate (209d) (80 mg, 69.6% yield) as a clear oil; MS (ES+): 487.20 (M+1).

### Step-4: Preparation of 2-(2-((2-(1-aminoisoquinolin-7-yl)-9H-fluoren-9-yl)oxy)phenyl)acetic acid (209e)

Compound 209e was prepared according to the procedure reported step-2 of scheme 1 from ethyl 2-(2-((2-(1-aminoisoquinolin-7-yl)-9H-fluoren-9-yl)oxy)phenyl)acetate (209d) (80 mg, 0.164 mmol) in MeOH/THF (6 mL, 1:1) using a solution of LiOH·H₂O (41.4 mg, 0.987 mmol) in water (2 mL) to afford after workup and purification using method-G, 2-(2-((2-(1-aminoisoquinolin-7-yl)-9H-fluoren-9-yl)oxy)phenyl)acetic acid (209e) (10 mg, 13.26% yield) HCl salt as a white solid; ¹H NMR (300 MHz, DMSO-d₆) δ 13.31 (s, 1H, D₂O exchangeable), 12.15 (s, 1H, D₂O exchangeable), 9.20 (s, 2H, D₂O exchangeable), 8.95 (s, 1H), 8.33 (d, J=8.5 Hz, 1H), 8.12-8.00 (m, 4H), 7.98 (d, J=7.5 Hz, 1H), 7.70 (d, J=6.9 Hz, 1H), 7.58-7.46 (m, 2H), 7.36 (t, J=7.9 Hz, 2H), 7.32-7.22 (m, 3H), 6.96 (t, J=7.4 Hz, 1H), 6.64 (s, 1H), 3.51 (s, 2H); MS (ES+): 459.10 (M+1); (ES−): 457.00 (M−1).

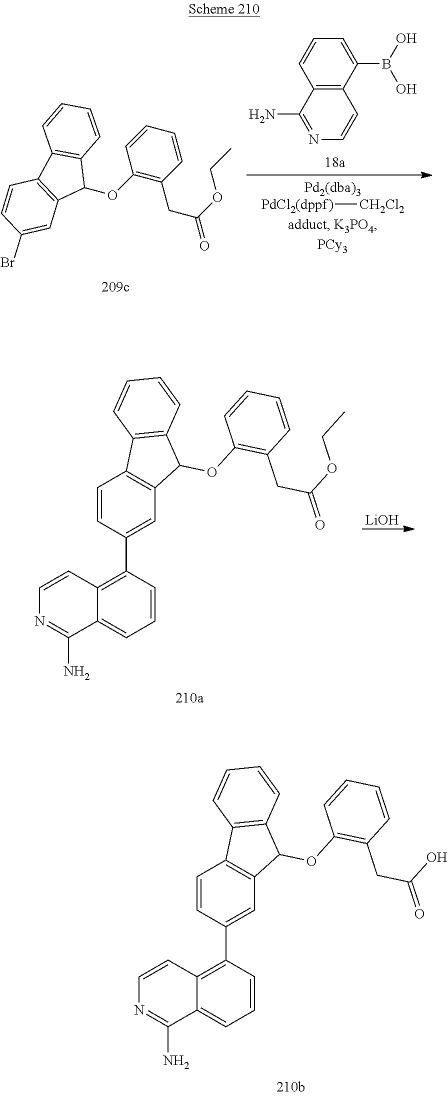

### Preparation of 2-(2-((2-(1-aminoisoquinolin-5-yl)-9H-fluoren-9-yl)oxy)phenyl)acetic acid (210b)

### Step-1: Preparation of ethyl 2-(2-((2-(1-aminoisoquinolin-5-yl)-9H-fluoren-9-yl)oxy)phenyl)acetate (210a)

Compound 210a was prepared according to the procedure reported in step-5 of scheme 1 from ethyl 2-(2-((2-bromo-9H-fluoren-9-yl)oxy)phenyl)acetate (209c) (100 mg, 0.236 mmol) in dioxane/THF (4 mL, 1:1) using (I-aminoisoquinolin-5-yl)boronic acid (18a) (66.6 mg, 0.354 mmol), K₃PO₄ (2 M aqueous solution, 0.472 mL, 0.945 mmol), PCy₃ (13.25 mg, 0.047 mmol), PdCl₂(dppf)-CH₂Cl₂ adduct (19.29 mg, 0.024 mmol) and Pd₂(dba)₃ (21.63 mg, 0.024 mmol) to afford after workup and purification using method-Y, ethyl 2-(2-((2-(1-aminoisoquinolin-5-yl)-9H-fluoren-9-yl)oxy)phenyl)acetate (210a) (80 mg, 69.6% yield) as a clear oil; ¹H NMR (300 MHz, DMSO-d₆) δ 8.21 (d, J=7.8 Hz, 1H), 8.01 (d, J=7.8 Hz, 1H), 7.95 (d, J=7.5 Hz, 1H), 7.73 (d, J=6.1 Hz, 1H), 7.56-7.45 (m, 7H), 7.38-7.21 (m, 3H), 6.94 (t, J=7.4 Hz, 1H), 6.88 (s, 2H), 6.77 (d, J=6.1 Hz, 1H), 6.68 (s, 1H), 3.79-3.60 (m, 2H), 3.50 (s, 2H), 0.81 (t, J=7.1 Hz, 3H); MS (ES+): 487.20 (M+1).

### Step-2: Preparation of 2-(2-((2-(1-aminoisoquinolin-5-yl)-9H-fluoren-9-yl)oxy)phenyl)acetic acid (210b)

Compound 210b was prepared according to the procedure reported step-2 of scheme 1 from ethyl 2-(2-((2-(1-aminoisoquinolin-5-yl)-9H-fluoren-9-yl)oxy)phenyl)acetate (210a) (80 mg, 0.164 mmol) in MeOH/THF (6 mL1:1) using a solution of LiOH·H₂O (41.4 mg, 0.987 mmol) in water (2 mL) to afford after workup and purification using method-G, 2-(2-((2-(1-aminoisoquinolin-5-yl)-9H-fluoren-9-yl)oxy)phenyl)acetic acid (210b) (8 mg, 10.61% yield) HCl salt as a white solid; ¹H NMR (300 MHz, DMSO-d₆) δ 13.16 (s, 1H, D₂O exchangeable), 12.07 (s, 1H, D₂O exchangeable), 9.10 (s, 2H, D₂O exchangeable), 8.59 (d, J=8.3 Hz, 1H), 8.07 (d, J=7.8 Hz, 1H), 8.02-7.91 (m, 2H), 7.84 (t, J=7.8 Hz, 1H), 7.62-7.49 (m, 5H), 7.43-7.31 (m, 2H), 7.29-7.20 (m, 2H), 6.99-6.90 (m, 2H), 6.65 (s, 1H), 3.48 (s, 2H); MS (ES+): 459.10 (M+1); MS (ES−): 457.10 (M−1).

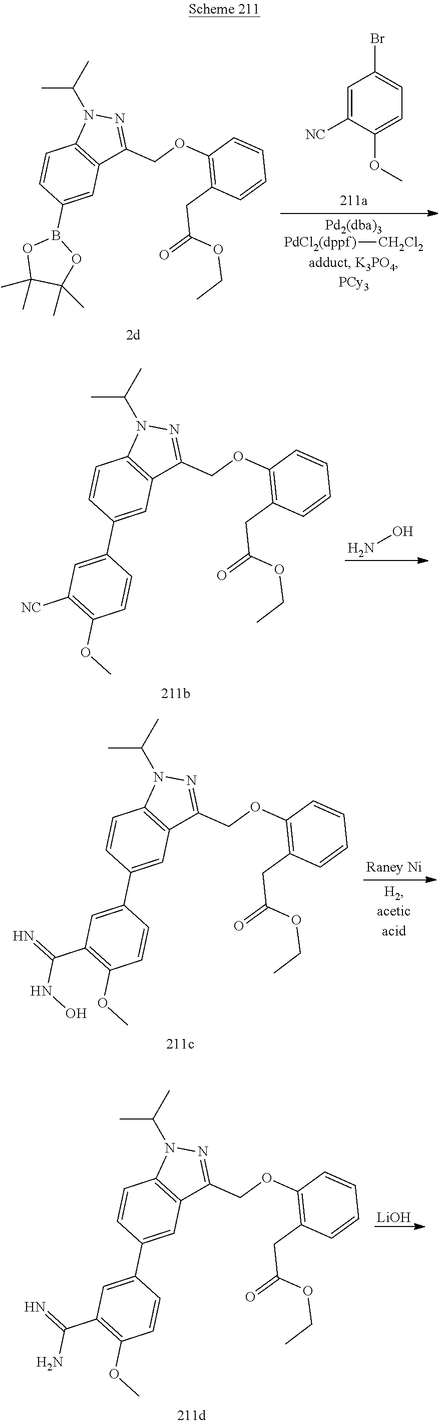

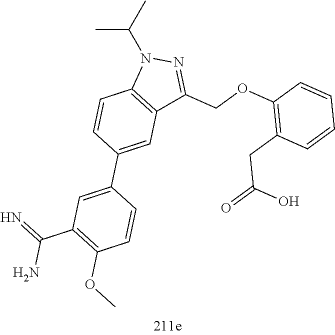 [SMILES: COc1ccc(-c2ccc3c(c2)c(COc2ccccc2CC(=O)O)nn3C(C)C)cc1C(=N)N]

### Preparation of 2-(2-((5-(3-carbamimidoyl-4-methoxyphenyl)-1-isopropyl-1H-indazol-3-yl)methoxy)phenyl)acetic acid (211e)

### Step-1: Preparation of ethyl 2-(2-((5-(3-cyano-4-methoxyphenyl)-1-isopropyl-1H-indazol-3-yl)methoxy)phenyl)acetate (211b)

Compound 211b was prepared according to the procedure reported in step-5 of scheme 1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (6 mL, Ratio: 1:1) using 5-bromo-2-methoxybenzonitrile (211a) (332 mg, 1.568 mmol; CAS #144649-99-0), K₃PO₄ (2 M aqueous solution, 2.090 mL, 4.18 mmol), PCy₃ (58.6 mg, 0.209 mmol), PdCl₂(dppf)-CH₂Cl₂ adduct (85 mg, 0.105 mmol) and Pd₂(dba)₃ (96 mg, 0.105 mmol) to afford after workup and purification using method-P, ethyl 2-(2-((5-(3-cyano-4-methoxyphenyl)-1-isopropyl-1H-indazol-3-yl)methoxy)phenyl)acetate (211b) (340 mg, 67.3% yield) as a clear oil; ¹H NMR (300 MHz, DMSO-d₆) δ 8.13-7.97 (m, 3H), 7.86-7.65 (m, 2H), 7.39-7.23 (m, 3H), 7.19 (d, J=7.4 Hz, 1H), 6.97-6.84 (m, 1H), 5.42 (s, 2H), 5.03 (p, J=6.6 Hz, 1H), 3.95 (s, 3H), 3.68 (q, J=7.1 Hz, 2H), 3.53 (s, 2H), 1.50 (d, J=6.5 Hz, 6H), 0.79 (t, J=7.0 Hz, 3H); MS (ES+): 484.15 (M+1).

### Step-2: Preparation of ethyl 2-(2-((5-(3-(N-hydroxycarbamimidoyl)-4-methoxyphenyl)-1-isopropyl-1H-indazol-3-yl)methoxy)phenyl)acetate (211c)

Compound 211c was prepared according to the procedure reported in step-5 of scheme 23 from ethyl 2-(2-((5-(3-cyano-4-methoxyphenyl)-1-isopropyl-1H-indazol-3-yl)methoxy)phenyl)acetate (211b) (340 mg, 0.703 mmol) in EtOH (10 mL) using hydroxylamine (0.464 mL, 7.03 mmol) to afford after workup and purification using method-E, ethyl 2-(2-((5-(3-(N-hydroxycarbamimidoyl)-4-methoxyphenyl)-1-isopropyl-1H-indazol-3-yl)methoxy)phenyl)acetate (211c) (250 mg, 68.8% yield) as a white solid; ¹H NMR (300 MHz, DMSO-d₆) δ 9.44 (s, 1H), 7.90 (s, 1H), 7.77 (d, J=8.8 Hz, 1H), 7.73-7.62 (m, 3H), 7.28 (d, J=3.9 Hz, 2H), 7.23-7.10 (m, 2H), 6.91 (t, J=6.4 Hz, 1H), 5.66 (s, 2H), 5.42 (s, 2H), 5.03 (q, J=6.6 Hz, 1H), 3.84 (s, 3H), 3.67 (q, J=7.1 Hz, 2H), 3.51 (s, 2H), 1.50 (d, J=6.5 Hz, 6H), 0.77 (t, J=7.1 Hz, 3H); MS (ES+): 517.25 (M+1).

### Step-3: Preparation of ethyl 2-(2-((5-(3-carbamimidoyl-4-methoxyphenyl)-1-isopropyl-1H-indazol-3-yl)methoxy)phenyl)acetate (211d)

Compound 211d was prepared according to the procedure reported in step-1 of scheme 24 from ethyl 2-(2-((5-(3-(N-hydroxycarbamimidoyl)-4-methoxyphenyl)-1-isopropyl-1H-indazol-3-yl)methoxy)phenyl)acetate (211c) (250 mg, 0.484 mmol) in EtOH (10 mL) using acetic acid (0.028 mL, 0.484 mmol), Raney Nickel (0.484 mmol) and hydrogen balloon (1 atm) to afford after workup and purification using method-AI, ethyl 2-(2-((5-(3-carbamimidoyl-4-methoxyphenyl)-1-isopropyl-1H-indazol-3-yl)methoxy)phenyl)acetate (211d) (200 mg, 83% yield) as a colorless oil; MS (ES+): 501.20 (M+1).

### Step-4: Preparation of 2-(2-((5-(3-carbamimidoyl-4-methoxyphenyl)-1-isopropyl-1H-indazol-3-yl)methoxy)phenyl)acetic acid (211e)

Compound 211e was prepared according to the procedure reported step-2 of scheme 1 from ethyl 2-(2-((5-(3-carbamimidoyl-4-methoxyphenyl)-1-isopropyl-1H-indazol-3-yl)methoxy)phenyl)acetate (211d) (200 mg, 0.400 mmol) in MeOH/THF (6 mL, 1:1) using a solution of LiOH·H₂O (101 mg, 2.397 mmol) in water (2 mL) to afford after workup and purification using method-G, 2-(2-((5-(3-carbamimidoyl-4-methoxyphenyl)-1-isopropyl-1H-indazol-3-yl)methoxy)phenyl)acetic acid (211e) (85 mg, 45% yield) HCl salt as a white solid; ¹H NMR (300 MHz, DMSO-d₆) δ 12.07 (s, 1H, D₂O exchangeable), 9.21 (s, 2H, D₂O exchangeable), 8.99 (m, 2H, D₂O exchangeable), 8.06 (s, 1H), 8.01-7.93 (m, 1H), 7.89-7.82 (m, 1H), 7.82-7.72 (m, 2H), 7.33 (d, J=8.9 Hz, 1H), 7.30-7.24 (m, 2H), 7.20 (d, J=7.4 Hz, 1H), 6.97-6.86 (m, 1H), 5.45 (s, 2H), 5.11-4.92 (m, 1H), 3.91 (s, 3H), 3.52 (s, 2H), 1.51 (d, J=6.5 Hz, 6H); MS (ES+): 473.20 (M+1); (ES−): 471.10 (M−1).

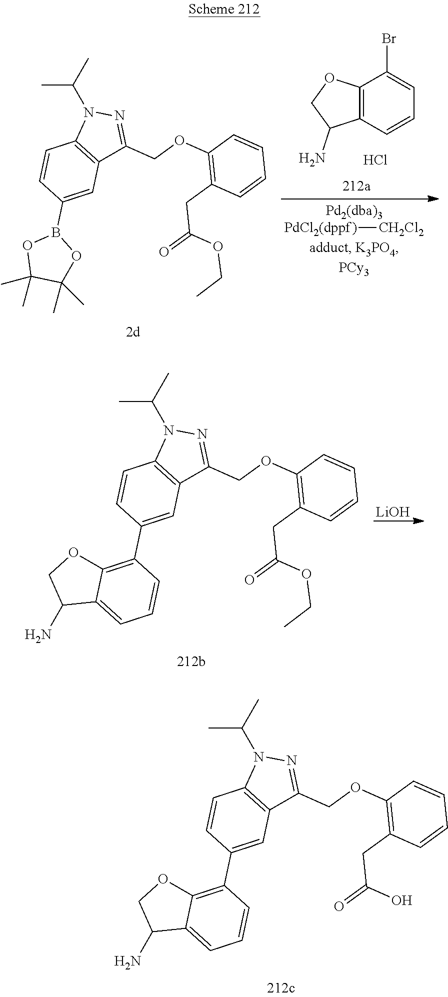

### Preparation of 2-(2-((5-(3-amino-2,3-dihydrobenzofuran-7-yl)-1-isopropyl-1H-indazol-3-yl)methoxy)phenyl)acetic acid (212c)

### Step-1: Preparation of ethyl 2-(2-((5-(3-amino-2,3-dihydrobenzofuran-7-yl)-1-isopropyl-1H-indazol-3-yl)methoxy)phenyl)acetate (212b)

Compound 212b was prepared according to the procedure reported in step-5 of scheme 1 from ethyl 2-(2-((1-isopropyl-5-(4,4,5,5-tetramethyl-1,3,2-dioxaborolan-2-yl)-1H-indazol-3-yl)methoxy)phenyl)acetate acid (2d) (200 mg, 0.418 mmol) in dioxane/THF (4 mL, 1:1) using 7-chloro-2,3-dihydrobenzofuran-3-amine hydrochloride (212a) (129 mg, 0.627 mmol; CAS #1384265-56-8), K₃PO₄ (2 M aqueous solution, 0.836 mL, 1.672 mmol), PCy₃ (23.45 mg, 0.084 mmol), PdCl₂(dppf)-CH₂Cl₂ adduct (34.1 mg, 0.042 mmol) and Pd₂(dba)₃ (38.3 mg, 0.042 mmol) to afford after workup and purification using method-E, ethyl 2-(2-((5-(3-amino-2,3-dihydrobenzofuran-7-yl)-1-isopropyl-1H-indazol-3-yl)methoxy)phenyl)acetate (212b) (180 mg, 89% yield) as a clear oil; MS (ES+): 486.20 (M+1); 508.20 (M+Na).

### Step-2: Preparation of 2-(2-((5-(3-amino-2,3-dihydrobenzofuran-7-yl)-1-isopropyl-1H-indazol-3-yl)methoxy)phenyl)acetic acid (212c)

Compound 212c was prepared according to the procedure reported step-2 of scheme 1 from ethyl 2-(2-((5-(3-amino-2,3-dihydrobenzofuran-7-yl)-1-isopropyl-1H-indazol-3-yl)methoxy)phenyl)acetate (212b) (180 mg, 0.371 mmol) in MeOH/THF (6 mL, 1:1) using a solution of LiOH·H₂O (93 mg, 2.224 mmol) in water (2 mL) to afford after workup and purification using method-G, 2-(2-((5-(3-amino-2,3-dihydrobenzofuran-7-yl)-1-isopropyl-1H-indazol-3-yl)methoxy)phenyl)acetic acid (212c) (40 mg, 23.58% yield) HCl salt as a white solid; ¹H NMR (300 MHz, DMSO-d₆) δ 12.14 (s, 1H, D₂O exchangeable), 8.81-8.60 (m, 3H, D₂O exchangeable), 8.10 (s, 1H), 7.82-7.70 (m, 2H), 7.59 (d, J=7.6 Hz, 2H), 7.29-7.23 (m, 2H), 7.20 (d, J=7.4 Hz, 1H), 7.10 (t, J=7.6 Hz, 1H), 6.97-6.82 (m, 1H), 5.44 (s, 2H), 5.16-5.06 (m, 1H), 5.01 (m, 1H), 4.82-4.69 (m, 1H), 4.65-4.49 (m, 1H), 3.56 (s, 2H), 1.51 (d, J=6.6 Hz, 6H); MS (ES+): 458.20 (M+1); (ES−): 456.10 (M−1).

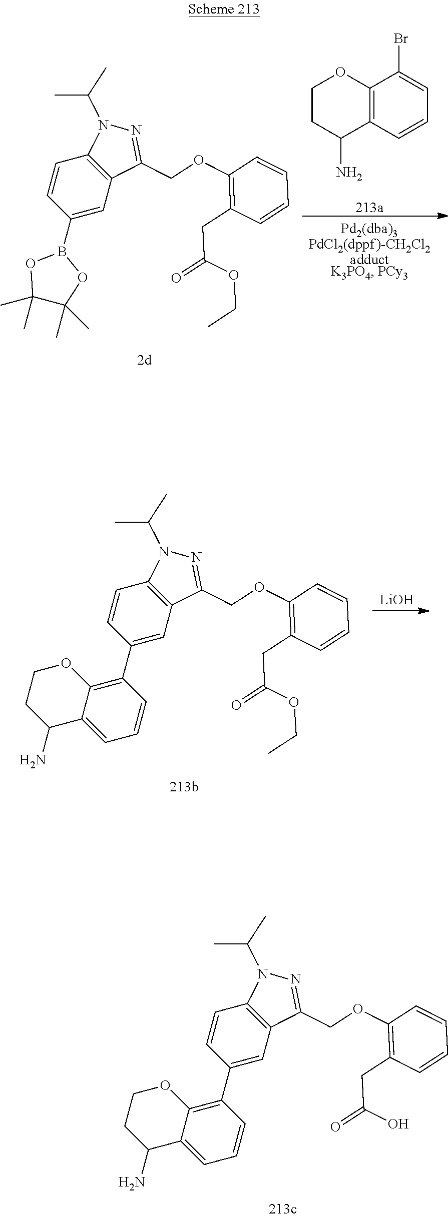

### Preparation of 2-(2-((5-(4-aminochroman-8-yl)-1-isopropyl-1H-indazol-3-yl)methoxy)phenyl)acetic acid (213c)

### Step-1: Preparation of ethyl 2-(2-((5-(4-aminochroman-8-yl)-1-isopropyl-1H-indazol-3-yl)methoxy)phenyl)acetate (213b)

Compound 213b was prepared according to the procedure reported in step-5 of scheme 1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 8-bromochroman-4-amine (213a) (143 mg, 0.627 mmol; CAS #886762-91-0), K₃PO₄ (2 M aqueous solution, 0.836 mL, 1.672 mmol), PCy₃ (23.45 mg, 0.084 mmol), PdCl₂(dppf)-CH₂Cl₂ adduct (34.1 mg, 0.042 mmol) and Pd₂(dba)₃ (38.3 mg, 0.042 mmol) to afford after workup and purification using method-AC, ethyl 2-(2-(5-(4-aminochroman-8-yl)-1-isopropyl-1H-indazol-3-yl)methoxy)phenyl)acetate (213b) (180 mg, 86% yield) as a clear oil; MS (ES+): 500.20 (M+1).

### Step-2: Preparation of Preparation of 2-(2-((5-(4-aminochroman-8-yl)-1-isopropyl-1H-indazol-3-yl)methoxy)phenyl)acetic acid (213c)

Compound 213c was prepared according to the procedure reported step-2 of scheme 1 from ethyl 2-(2-((5-(4-aminochroman-8-yl)-1-isopropyl-1H-indazol-3-yl)methoxy)phenyl)acetate (213b) (180 mg, 0.360 mmol) in MeOH/THF (6 mL, 1:1) using a solution of LiOH·H₂O (91 mg, 2.162 mmol) in water (2 mL) to afford after workup and purification using method-G, 2-(2-((5-(4-aminochroman-8-yl)-1-isopropyl-1H-indazol-3-yl)methoxy)phenyl)acetic acid (213c) (80 mg, 47.1% yield) HCl salt as a white solid; ¹H NMR (300 MHz, DMSO-d₆) δ12.13 (s, 1H, D₂O exchangeable), 8.81-8.57 (m, 3H, D₂O exchangeable), 7.84-7.76 (m, 1H), 7.72 (d, J=8.8 Hz, 1H), 7.60-7.43 (m, 2H), 7.38-7.30 (m, 1H), 7.29-7.22 (m, 2H), 7.22-7.14 (m, 1H), 7.06 (t, J=7.6 Hz, 1H), 6.95-6.84 (m, 1H), 5.43 (s, 2H), 5.01 (m, 1H), 4.62-4.48 (m, 1H), 4.30-4.16 (m, 2H), 3.52 (s, 2H), 2.40-2.24 (m, 1H), 2.24-2.09 (m, 1H), 1.51 (d, J=6.6 Hz, 6H); MS (ES+): 472.20 (M+1); (ES−): 470.10 (M−1).

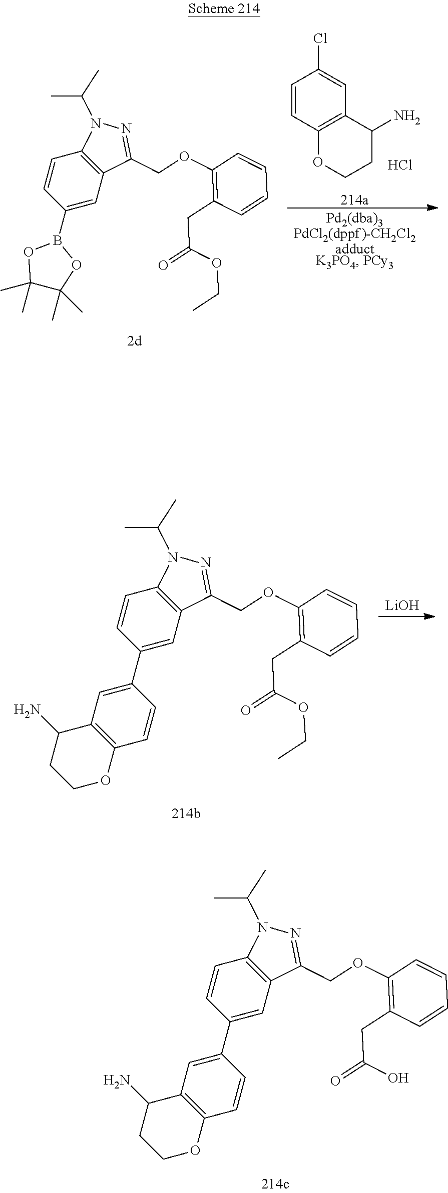

### Preparation of 2-(2-((5-(4-aminochroman-6-yl)-1-isopropyl-1H-indazol-3-yl)methoxy)phenyl)acetic acid (214c)

### Step-1: Preparation of ethyl 2-(2-((5-(4-aminochroman-6-yl)-1-isopropyl-1H-indazol-3-yl)methoxy)phenyl)acetate (214b)

Compound 214b was prepared according to the procedure reported in step-S of scheme 1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 6-chlorochroman-4-amine hydrochloride (214a) (138 mg, 0.627 mmol; CAS #191608-09-0), K₃PO₄ (2 M aqueous solution, 0.836 mL, 1.672 mmol), PCy₃ (23.45 mg, 0.084 mmol), PdCl₂(dppf)-CH₂Cl₂ adduct (34.1 mg, 0.042 mmol) and Pd₂(dba)₃ (38.3 mg, 0.042 mmol) to afford after workup and purification using method-AC, ethyl 2-(2-((5-(4-aminochroman-6-yl)-1-isopropyl-1H-indazol-3-yl)methoxy)phenyl)acetate (214b) (180 mg, 86% yield) as a clear oil; MS (ES+): 500.20 (M+1); 522.20 (M+Na).

### Step-2: Preparation of 2-(2-((5-(4-aminochroman-6-yl)-1-isopropyl-1H-indazol-3-yl)methoxy)phenyl)acetic acid (214c)

Compound 214c was prepared according to the procedure reported step-2 of scheme 1 from ethyl 2-(2-((5-(4-aminochroman-6-yl)-1-isopropyl-1H-indazol-3-yl)methoxy)phenyl)acetate (214b) (180 mg, 0.360 mmol) in MeOH/THF (6 mL, 1:1) using a solution of LiOH·H₂O (91 mg, 2.162 mmol) in water (2 mL) to afford after workup and purification using method-G, 2-(2-((5-(4-aminochroman-6-yl)-1-isopropyl-1H-indazol-3-yl)methoxy)phenyl)acetic acid (214c) (15 mg, 8.83% yield) HCl salt as a white solid; ¹H NMR (300 MHz, DMSO-d₆) δ 12.12 (s, 1H, D₂O exchangeable), 8.52 (s, 3H, D₂O exchangeable), 8.03-7.98 (m, 1H), 7.90-7.85 (m, 1H), 7.82 (m, 1H), 7.72 (m, 1H), 7.68-7.60 (m, 1H), 7.30-7.23 (m, 2H), 7.20 (d, J=7.4 Hz, 1H), 6.98-6.88 (m, 2H), 5.43 (s, 2H), 5.10-4.94 (m, 1H), 4.65-4.52 (m, 1H), 4.36-4.21 (m, 2H), 3.52 (s, 2H), 2.32-2.24 (m, 1H), 2.18-2.09 (m, 1H), 1.51 (d, J=6.5 Hz, 6H); MS (ES+): 472.20 (M+1); (ES−): 470.10 (M−1).

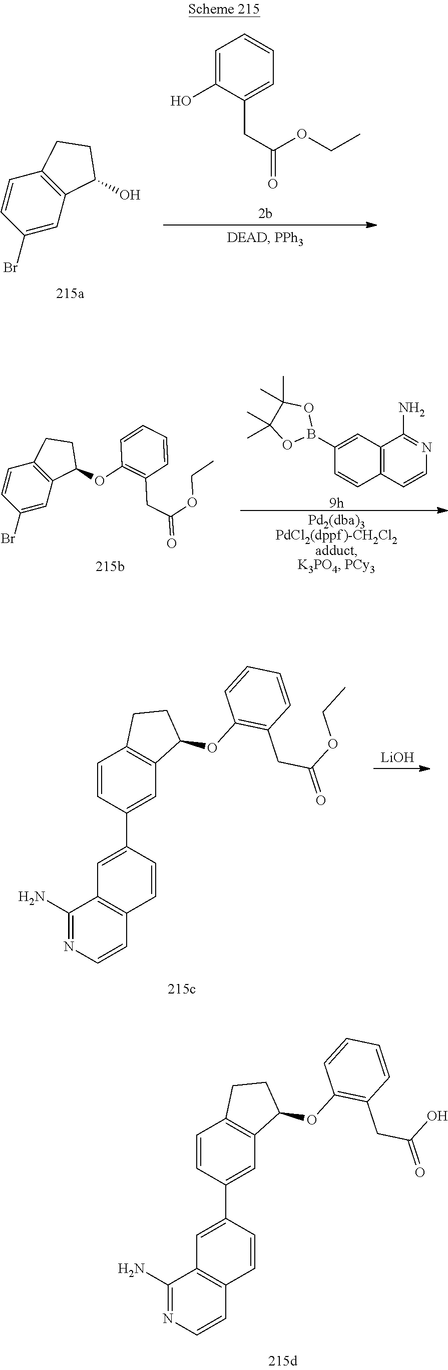

### Preparation of (R)-2-(2-((6-(1-aminoisoquinolin-7-yl)-2,3-dihydro-1H-inden-1-yl)oxy)phenyl)acetic acid (215d)

### Step-1: Preparation of (R)-ethyl 2-(2-((6-bromo-2,3-dihydro-1H-inden-1-yl)oxy)phenyl)acetate (215b)

Compound 215b was prepared according to the procedure reported in step-1 of scheme 8 from (S)-6-bromo-2,3-dihydro-1H-inden-1-ol (215a) (500 mg, 2.347 mmol; CAS #1096537-29-9) in DCM (10 mL) using ethyl 2-(2-hydroxyphenyl)acetate (2b) (846 mg, 4.69 mmol), PPh₃ (1231 mg, 4.69 mmol) and a solution of DEAD (817 mg, 4.69 mmol) in DCM to afford after workup and purification using method-L, (R)-ethyl 2-(2-((6-bromo-2,3-dihydro-1H-inden-1-yl)oxy)phenyl)acetate (215b) (360 mg, 40.9% yield) as a yellow oil; ¹H NMR (300 MHz, DMSO-d₆) δ 7.49 (dd, J=8.0, 2.0 Hz, 1H), 7.46-7.43 (m, 1H), 7.33-7.25 (m, 2H), 7.23-7.16 (m, 2H), 6.95-6.89 (m, 1H), 5.89-5.78 (m, 1H), 4.00-3.90 (m, 2H), 3.49 (s, 2H), 3.04-2.92 (m, 1H), 2.88-2.78 (m, 1H), 2.68-2.56 (m, 1H), 2.04-1.89 (m, 1H), 1.09-0.99 (m, 3H); MS (ES+): 375.00 & 377.00 (M+1); 397.00 & 399.00 (M+Na).

### Step-2: Preparation of (R)-ethyl 2-(2-((6-(1-aminoisoquinolin-7-yl)-2,3-dihydro-1H-inden-1-yl)oxy)phenyl)acetate (215c)

Compound 215c was prepared according to the procedure reported in step-5 of scheme 1 from (R)-ethyl 2-(2-((6-bromo-2,3-dihydro-1H-inden-1-yl)oxy)phenyl)acetate (215b) (180 mg, 0.480 mmol) in dioxane/THF (4 mL, 1:1) using 7-(4,4,5,5-tetramethyl-1,3,2-dioxaborolan-2-yl)isoquinolin-1-amine (9h) (194 mg, 0.720 mmol), K₃PO₄ (2 M aqueous solution, 0.959 mL, 1.919 mmol), PCy₃ (26.9 mg, 0.096 mmol), PdCl₂(dppf)-CH₂Cl₂ adduct (39.2 mg, 0.048 mmol) and Pd₂(dba)₃ (43.9 mg, 0.048 mmol) to afford after workup and purification using method-Y, (R)-ethyl 2-(2-((6-(1-aminoisoquinolin-7-yl)-2,3-dihydro-1H-inden-1-yl)oxy)phenyl)acetate acetate (215c) (120 mg, 57.0% yield) as a clear oil; ¹H NMR (300 MHz, DMSO-d₆) δ 8.51 (s, 1H), 7.97-7.90 (m, 1H), 7.85-7.73 (m, 4H), 7.46 (d, J=7.8 Hz, 1H), 7.36-7.25 (m, 2H), 7.25-7.17 (m, 1H), 6.98 (s, 2H), 6.95-6.88 (m, 2H), 5.97-5.86 (m, 1H), 3.78 (q, J=7.1 Hz, 2H), 3.50 (s, 2H), 3.15-3.01 (m, 1H), 3.01-2.87 (m, 1H), 2.76-2.60 (m, 1H), 2.09-1.93 (m, 1H), 0.79 (t, J=7.1 Hz, 3H); MS (ES+): 439.20 (M+1):

### Step-3: (R)-2-(2-((6-(1-aminoisoquinolin-7-yl)-2,3-dihydro-1H-inden-1-yl)oxy)phenyl)acetic acid (215d)

Compound 215d was prepared according to the procedure reported step-2 of scheme 1 from (R)-ethyl 2-(2-((6-(1-aminoisoquinolin-7-yl)-2,3-dihydro-1H-inden-1-yl)oxy)phenyl)acetate acetate (215c) (120 mg, 0.274 mmol) in MeOH/THF (6 mL, 1:1) using a solution of LiOH·H₂O (68.9 mg, 1.642 mmol) in water (2 mL) to afford after workup and purification using method-G, (R)-2-(2-((6-(1-aminoisoquinolin-7-yl)-2,3-dihydro-1H-inden-1-yl)oxy)phenyl)acetic acid (215d) (32 mg, 28.5% yield) HCl salt as a white solid; ¹H NMR (300 MHz, DMSO-d₆) δ 12.15 (s, 1H), 8.52 (s, 1H), 7.97 (dd, J=8.5, 1.7 Hz, 1H), 7.87-7.73 (m, 4H), 7.46 (d, J=7.8 Hz, 1H), 7.36-7.17 (m, 3H), 7.04 (s, 2H), 6.98-6.86 (m, 2H), 5.90 (t, J=5.7 Hz, 1H), 3.47 (s, 2H), 3.16-3.03 (m, 1H), 3.01-2.85 (m, 1H), 2.77-2.57 (m, 1H), 2.11-1.95 (m, 1H); MS (ES+): 411.20 (M+1); MS (ES−): 409.10 (M−1).

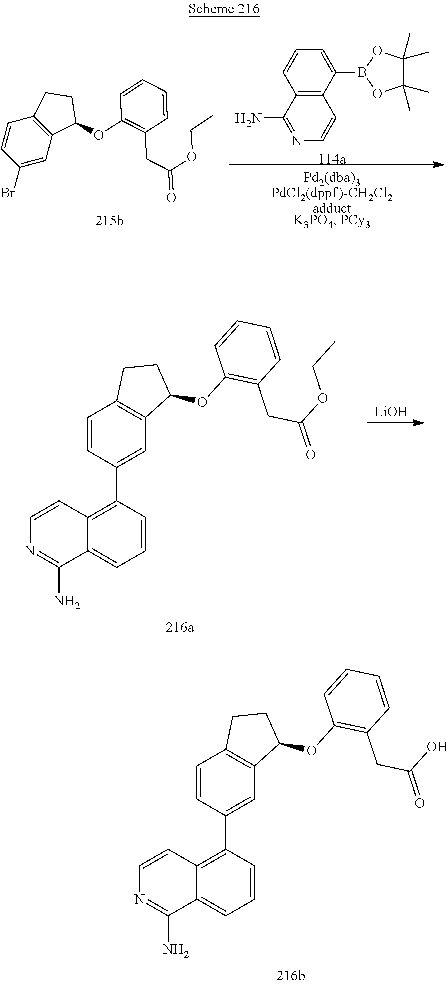

### Preparation of (R)-2-(2-((6-(1-aminoisoquinolin-5-yl)-2,3-dihydro-1H-inden-1-yl)oxy)phenyl)acetic acid (216b)

### Step-1: Preparation of (R)-ethyl 2-(2-((6-(1-aminoisoquinolin-5-yl)-2,3-dihydro-1H-inden-1-yl)oxy)phenyl)acetate (216a)

Compound 216a was prepared according to the procedure reported in step-5 of scheme 1 from (R)-ethyl 2-(2-((6-bromo-2,3-dihydro-1H-inden-1-yl)oxy)phenyl)acetate (215b) (180 mg, 0.480 mmol) in dioxane/THF (4 mL, 1:1) using 5-(4,4,5,5-tetramethyl-1,3,2-dioxaborolan-2-yl)isoquinolin-1-amine (114a) (194 mg, 0.72 mmol), K₃PO₄ (2 M aqueous solution, 0.959 mL, 1.919 mmol), PCy₃ (26.9 mg, 0.096 mmol), PdCl₂(dppf)-CH₂Cl₂ adduct (39.2 mg, 0.048 mmol) and Pd₂(dba)₃ (43.9 mg, 0.048 mmol) to afford after workup and purification using method-Y, (R)-ethyl 2-(2-((6-(1-aminoisoquinolin-5-yl)-2,3-dihydro-1H-inden-1-yl)oxy)phenyl)acetate (216a) (140 mg, 66.6% yield) as a clear oil; ¹H NMR (300 MHz, DMSO-d₆) δ 8.26-8.15 (m, 1H), 7.73 (d, J=6.1 Hz, 1H), 7.53-7.49 (m, 2H), 7.46 (d, J=7.8 Hz, 1H), 7.40-7.35 (m, 1H), 7.35-7.32 (m, 1H), 7.32-7.22 (m, 2H), 7.22-7.16 (m, 1H), 6.94-6.89 (m, 1H), 6.87 (s, 2H), 6.78-6.74 (m, 1H), 5.91 (t, J=5.8 Hz, 1H), 3.80-3.55 (m, 2H), 3.47 (d, J=5.6 Hz, 2H), 3.19-3.05 (m, 1H), 3.05-2.89 (m, 1H), 2.79-2.64 (m, 1H), 2.11-1.97 (m, 1H), 0.78 (t, J=7.1 Hz, 3H); MS (ES+): 439.20 (M+1); (ES−): 437.10 (M−1).

### Step-2: Preparation of (R)-2-(2-((6-(1-aminoisoquinolin-5-yl)-2,3-dihydro-1H-inden-1-yloxy)phenyl)acetic acid (216b)

Compound 216b was prepared according to the procedure reported step-2 of scheme 1 from (R)-ethyl 2-(2-((6-(1-aminoisoquinolin-5-yl)-2,3-dihydro-1H-inden-1-yl)oxy)phenyl)acetate (216a) (120 mg, 0.274 mmol) in MeOH/THF (6 mL, 1:1) using a solution of LiOH·H₂O (68.9 mg, 1.642 mmol) in water (2 mL) to afford after workup and purification using method-G, (R)-2-(2-((6-(1-aminoisoquinolin-5-yl)-2,3-dihydro-1H-inden-1-yl)oxy)phenyl)acetic acid (216b) (25 mg, 22.26% yield) HCl salt as a white solid; ¹H NMR (300 MHz, DMSO-d₆) δ 12.05 (s, 1H, D₂O exchangeable), 8.25-8.16 (m, 1H), 7.73 (d, J=6.1 Hz, 1H), 7.55-7.49 (m, 2H), 7.49-7.43 (m, 1H), 7.40-7.33 (m, 2H), 7.29-7.22 (m, 2H), 7.22-7.15 (m, 1H), 6.95-6.84 (m, 3H, 2H D₂O exchangeable), 6.79-6.73 (m, 1H), 5.91 (t, J=5.8 Hz, 1H), 3.43 (s, 2H), 3.21-3.07 (m, 1H), 3.04-2.88 (m, 1H), 2.76-2.61 (m, 1H), 2.15-1.98 (m, 1H); MS (ES+): 411.10 (M+1); (ES−): 409.10 (M−1).

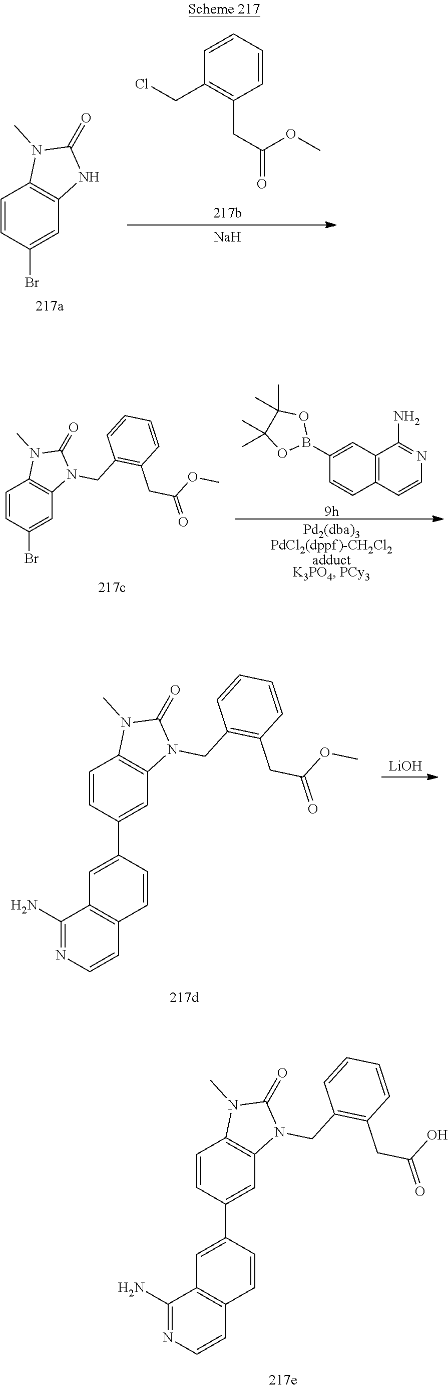

### Preparation of 2-(2-((6-(1-aminoisoquinolin-7-yl)-3-methyl-2-oxo-2,3-dihydro-1H-benzo[d]imidazol-1-yl)methyl)phenyl)acetic acid (217e)

### Step-1: Preparation of methyl 2-(2-((6-bromo-3-methyl-2-oxo-2,3-dihydro-1H-benzo[d]imidazol-1-yl)methyl)phenyl)acetate (217c)

Compound 217c was prepared according to the procedure reported in step-1 of scheme 1 from 5-bromo-1-methyl-1H-benzo[d]imidazol-2(3H)-one (217a) (500 mg, 2.202 mmol; CAS #84712-08-3) in DMF (15 mL) using sodium hydride (132 mg, 3.30 mmol) and methyl 2-(2-(chloromethyl)phenyl)acetate (217b) (437 mg, 2.202 mmol; CAS #95360-33-1) to afford after workup and purification using method-J, methyl 2-(2-((6-bromo-3-methyl-2-oxo-2,3-dihydro-1H-benzo[d]imidazol-1-yl)methyl)phenyl)acetate (217c) (360 mg, 42.0% yield) as a white solid; ¹H NMR (300 MHz, DMSO-d₆) δ 7.30-7.24 (m, 2H), 7.24-7.19 (m, 2H), 7.19-7.16 (m, 1H), 7.16-7.10 (m, 1H), 6.96-6.88 (m, 1H), 5.07 (s, 2H), 3.93 (s, 2H), 3.57 (s, 3H), 3.37 (s, 3H); MS (ES+): 389.00 & 411.00 (M+1).

### Step-2: Preparation of methyl 2-(2-((6-(1-aminoisoquinolin-7-yl)-3-methyl-2-oxo-2,3-dihydro-1H-benzo[d]imidazol-1-yl)methyl)phenyl)acetate (217d)

Compound 217d was prepared according to the procedure reported in step-5 of scheme 1 from methyl 2-(2-((6-bromo-3-methyl-2-oxo-2,3-dihydro-1H-benzo[d]imidazol-1-yl)methyl)phenyl)acetate (217c) (180 mg, 0.462 mmol) in dioxane/THF (4 mL, 1:1) using 7-(4,4,5,5-tetramethyl-1,3,2-dioxaborolan-2-yl)isoquinolin-1-amine (9h) (187 mg, 0.694 mmol), K₃PO₄ (2 M aqueous solution, 0.925 mL, 1.850 mmol), PCy₃ (25.9 mg, 0.092 mmol), PdCl₂(dppf)-CH₂Cl₂ adduct (37.8 mg, 0.046 mmol) and Pd₂(dba)₃ (42.3 mg, 0.046 mmol) to afford after workup and purification using method-Y, methyl 2-(2-((6-(1-aminoisoquinolin-7-yl)-3-methyl-2-oxo-2,3-dihydro-1H-benzo[d]imidazol-1-yl)methyl)phenyl)acetate (217d) (120 mg, 57.3% yield) as a clear oil; ¹H NMR (300 MHz, DMSO-d₆) δ 8.46-8.38 (m, 1H), 7.90-7.83 (m, 1H), 7.80-7.69 (m, 2H), 7.64-7.58 (m, 1H), 7.46 (d, J=1.7 Hz, 1H), 7.33 (d, J=8.2 Hz, 1H), 7.30-7.24 (m, 1H), 7.24-7.18 (m, 2H), 7.06-7.00 (m, 1H), 6.91 (s, 1H), 6.89 (s, 2H), 5.16 (s, 2H), 4.00 (s, 2H), 3.60 (s, 3H), 3.44 (s, 3H); MS (ES+): 453.20 (M+1); (ES−): 451.10 (M−1).

### Step-3: Preparation of 2-(2-((6-(1-aminoisoquinolin-7-yl)-3-methyl-2-oxo-2,3-dihydro-1H-benzo[d]imidazol-1-yl)methyl)phenyl)acetic acid (217e)

Compound 217e was prepared according to the procedure reported step-2 of scheme 1 from methyl 2-(2-((6-(1-aminoisoquinolin-7-yl)-3-methyl-2-oxo-2,3-dihydro-1H-benzo[d]imidazol-1-yl)methyl)phenyl)acetate (217d) (120 mg, 0.265 mmol) in MeOH/THF (6 mL, 1:1) using a solution of LiOH·H₂O (66.8 mg, 1.591 mmol) in water (2 mL) to afford after workup and purification using method-G, 2-(2-((6-(1-aminoisoquinolin-7-yl)-3-methyl-2-oxo-2,3-dihydro-1H-benzo[d]imidazol-1-yl)methyl)phenyl)acetic acid (217e) (50 mg, 43% yield) HCl salt as a white solid; ¹H NMR (300 MHz, DMSO-d₆) δ 12.91 (s, 1H, DO exchangeable), 9.16 (s, 2H, D₂O exchangeable), 8.92 (s, 1H), 8.20 (dd. J=8.6, 1.6 Hz, 1H), 7.96 (d, J=8.5 Hz, 1H), 7.67 (t, J=7.4 Hz, 2H), 7.59 (s, 1H), 7.37 (d, J=8.2 Hz, 1H), 7.27 (d, J=7.2 Hz, 1H), 7.23-7.09 (m, 3H), 6.92 (d, J=7.4 Hz, 1H), 5.21 (s, 2H), 3.93 (s, 2H), 3.45 (s, 3H); MS (ES+): 439.10 (M+1); MS (ES−): 437.10 (M−1).

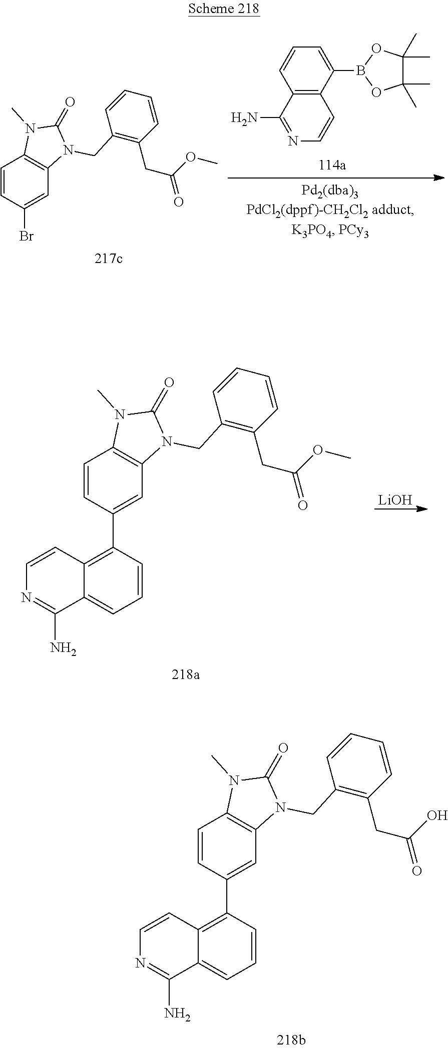 [SMILES: COC(=O)Cc1ccccc1Cn1c(=O)n(C)c2ccc(Br)cc21 ; CC1(C)OB(c2cccc3c(N)nccc23)OC1(C)C ; COC(=O)Cc1ccccc1Cn1c(=O)n(C)c2ccc(-c3cccc4c(N)nccc34)cc21 ; "LiOH" ; Cn1c(=O)n(Cc2ccccc2CC(=O)O)c2cc(-c3cccc4c(N)nccc34)ccc21]

### Preparation of 2-(2-((6-(1-aminoisoquinolin-5-yl)-3-methyl-2-oxo-2,3-dihydro-1H-benzo[d]imidazol-1-yl)methyl)phenyl)acetic acid (218b)

### Step-1: Preparation of methyl 2-(2-((6-(1-aminoisoquinolin-5-yl)-3-methyl-2-oxo-2,3-dihydro-1H-benzo[d]imidazol-1-yl)methyl)phenyl)acetate (218a)

Compound 218a was prepared according to the procedure reported in step-5 of scheme 1 from methyl 2-(2-((6-bromo-3-methyl-2-oxo-2,3-dihydro-1H-benzo[d]imidazol-1-yl)methyl)phenyl)acetate (217c) (180 mg, 0.462 mmol) in dioxane/THF (4 mL, 1:1) using 5-(4,4,5,5-tetramethyl-1,3,2-dioxaborolan-2-yl)isoquinolin-1-amine (114a) (187 mg, 0.694 mmol), K₃PO₄ (2 M aqueous solution, 0.925 mL, 1.850 mmol), PCy₃ (25.9 mg, 0.092 mmol), PdCl₂(dppf)-CH₂Cl₂ adduct (37.8 mg, 0.046 mmol) and Pd₂(dba)₃ (42.3 mg, 0.046 mmol) to afford after workup and purification using method-Y, methyl 2-(2-((6-(1-aminoisoquinolin-5-yl)-3-methyl-2-oxo-2,3-dihydro-1H-benzo[d]imidazol-1-yl)methyl)phenyl)acetate (218a) (150 mg, 71.7% yield) as a clear oil; ¹H NMR (300 MHz, DMSO-d₆) ¿ 8.21-8.11 (m, 1H), 7.63 (d, J=6.1 Hz, 1H), 7.51-7.40 (m, 2H), 7.31 (d, J=8.0 Hz, 1H), 7.27-7.23 (m, 2H), 7.23-7.16 (m, 1H), 7.12 (dd, J=8.0, 1.6 Hz, 1H), 7.01 (d, J=7.0 Hz, 1H), 6.97 (d, J=1.5 Hz, 1H), 6.83 (s, 2H), 6.61 (dd, J=6.1, 0.8 Hz, 1H), 5.12 (s, 2H), 3.89 (s, 2H), 3.46 (s, 3H), 3.45 (s, 3H); MS (ES+): 453.20 (M+1).

### Step-2: Preparation of 2-(2-((6-(1-aminoisoquinolin-5-yl)-3-methyl-2-oxo-2,3-dihydro-1H-benzo[d]imidazol-1-yl)methyl)phenyl)acetic acid (218b)

Compound 218b was prepared according to the procedure reported step-2 of scheme 1 from methyl 2-(2-((6-(1-aminoisoquinolin-5-yl)-3-methyl-2-oxo-2,3-dihydro-1H-benzo[d]imidazol-1-yl)methyl)phenyl)acetate (218a) (150 mg, 0.331 mmol) in MeOH/THF (6 mL, 1:1) using a solution of LiOH·H₂O (83 mg, 1.989 mmol) in water (2 mL) to afford after workup and purification using method-G, 2-(2-((6-(1-aminoisoquinolin-5-yl)-3-methyl-2-oxo-2,3-dihydro-1H-benzo[d]imidazol-1-yl)methyl)phenyl)acetic acid (218b) (85 mg, 58.5% yield) HCl salt as a white solid; ¹H NMR (300 MHz, DMSO-d₆) δ 13.43 (s, 1H, D₂O exchangeable), 12.42 (s, 1H, D₂O exchangeable), 9.21 (s, 2H, D₂O exchangeable), 8.69-8.44 (m, 1H), 7.84-7.72 (m, 2H), 7.53 (d, J=7.3 Hz, 1H), 7.36 (d, J=8.1 Hz, 1H), 7.27-7.20 (m, 2H), 7.20-7.15 (m, 1H), 7.15-7.09 (m, 1H), 7.03-6.95 (m, 1H), 6.93-6.86 (m, 1H), 6.81 (d, J=7.2 Hz, 1H), 5.13 (s, 2H), 3.81 (s, 2H), 3.46 (s, 3H); MS (ES+): 439.10 (M+1); (ES−): 437.10 (M−1).

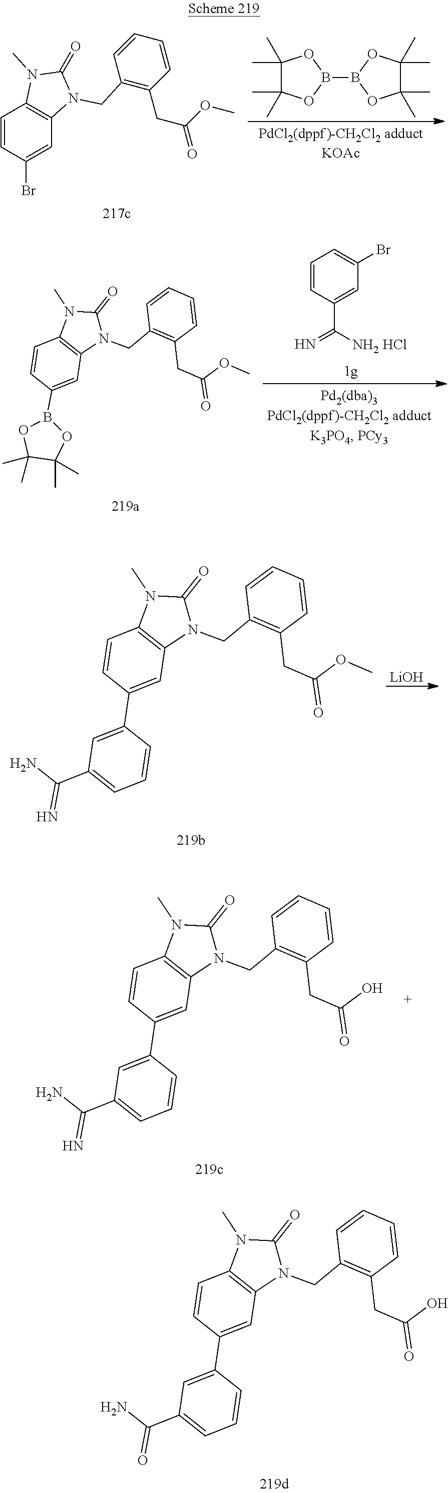

### Preparation of 2-(2-((6-(3-carbamimidoylphenyl)-3-methyl-2-oxo-2,3-dihydro-1H-benzo[d]imidazol-1-yl)methyl)phenyl)acetic acid (219c) and 2-(2-((6-(3-carbamoylphenyl)-3-methyl-2-oxo-2,3-dihydro-1H-benzo[d]imidazol-1-yl)methyl)phenyl)acetic acid (219d)

### Step-1: Preparation of methyl 2-(2-((3-methyl-2-oxo-6-(4,4,5,5-tetramethyl-1,3,2-dioxaborolan-2-yl)-2,3-dihydro-1H-benzo[d]imidazol-1-yl)methyl)phenyl)acetate (219a)

Compound 219a was prepared according to the procedure reported in step-4 of scheme 1 from methyl 2-(2-((6-bromo-3-methyl-2-oxo-2,3-dihydro-1H-benzo[d]imidazol-1-yl)methyl)phenyl)acetate (217c) (360 mg, 0.925 mmol) in anhydrous dioxane (20 mL) using BISPIN (470 mg, 1.850 mmol), KOAc (227 mg, 2.312 mmol) and PdCl₂(dppf)-CH₂Cl₂ adduct (45.3 mg, 0.055 mmol) to afford after workup and purification using method-L, methyl 2-(2-((3-methyl-2-oxo-6-(4,4,5,5-tetramethyl-1,3,2-dioxaborolan-2-yl)-2,3-dihydro-1H-benzo[d]imidazol-1-yl)methyl)phenyl)acetate (219a) (340 mg, 84% yield) as a clear oil; MS (ES+): 437.20 (M+1); 459.20 (M+Na).

### Step-2: Preparation of methyl 2-(2-((6-(3-carbamimidoylphenyl)-3-methyl-2-oxo-2,3-dihydro-1H-benzo[d]imidazol-1-yl)methyl)phenyl)acetate (219b)

Compound 219b was prepared according to the procedure reported in step-5 of scheme 1 from methyl 2-(2-((3-methyl-2-oxo-6-(4,4,5,5-tetramethyl-1,3,2-dioxaborolan-2-yl)-2,3-dihydro-1H-benzo[d]imidazol-1-yl)methyl)phenyl)acetate (219a) (340 mg, 0.779 mmol) in dioxane/THF (4 mL, 1:1) using 3-bromobenzimidamide hydrochloride (1g) (367 mg, 1.559 mmol), K₃PO₄ (2 M aqueous solution, 1.559 mL, 3.12 mmol), PCy₃ (43.7 mg, 0.156 mmol). PdCl₂(dppf)-CH₂Cl₂ adduct (63.6 mg, 0.078 mmol) and Pd₂(dba)₃ (71.4 mg, 0.078 mmol) to afford after workup and purification using method-AI, methyl 2-(2-((6-(3-carbamimidoylphenyl)-3-methyl-2-oxo-2,3-dihydro-1H-benzo[d]imidazol-1-yl)methyl)phenyl)acetate (219b) (200 mg, 59.9% yield) as a clear oil; MS (ES+): 429.20 (M+1); (ES−): 427.10 (M−1).

### Step-3: Preparation of 2-(2-((6-(3-carbamimidoylphenyl)-3-methyl-2-oxo-2,3-dihydro-1H-benzo[d]imidazol-1-yl)methyl)phenyl)acetic acid (219c) and 2-(2-((6-(3-carbamoylphenyl)-3-methyl-2-oxo-2,3-dihydro-1H-benzo[d]imidazol-1-yl)methyl)phenyl)acetic acid (219d)

Compounds 219c and 219d were prepared according to the procedure reported in step-2 of scheme 1 from methyl 2-(2-((6-(3-carbamimidoylphenyl)-3-methyl-2-oxo-2,3-dihydro-1H-benzo[d]imidazol-1-yl)methyl)phenyl)acetate (219b) (200 mg, 0.467 mmol) in MeOH/THF (6 mL, 1:1) using a solution of LiOH·H₂O (118 mg, 2.80 mmol) in water (2 mL) to afford after workup and purification using method-G, 2-(2-((6-(3-carbamimidoylphenyl)-3-methyl-2-oxo-2,3-dihydro-1H-benzo[d]imidazol-1-yl)methyl)phenyl)acetic acid (219c) (22 mg, 11.37% yield) HCl salt as a white solid; ¹H NMR (300 MHz, DMSO-d₆) δ 10.02 (s, 2H, D₂O exchangeable), 8.91 (s, 2H, D₂O exchangeable), 8.21 (s, 1H), 7.97-7.89 (m, 1H), 7.75-7.68 (m, 1H), 7.65-7.56 (m, 2H), 7.56-7.49 (m, 1H), 7.30 (d, J=8.2 Hz, 1H), 7.25-7.19 (m, 1H), 7.19-7.04 (m, 2H), 6.90-6.80 (m, 1H), 5.17 (s, 2H), 3.73 (s, 2H), 3.45 (s, 3H); MS (ES+): 415.20 (M+1); (ES−): 413.10 (M−1) and 2-(2-((6-(3-carbamoylphenyl)-3-methyl-2-oxo-2,3-dihydro-1H-benzo[d]imidazol-1-yl)methyl)phenyl)acetic acid (219d) (10 mg, 5.16% yield) free base as a white solid; ¹H NMR (300 MHz, DMSO-d₆) δ 12.50 (s, 1H, D₂O exchangeable), 8.10-8.02 (m, 2H, 1H D₂O exchangeable), 7.81-7.74 (m, 1H), 7.74-7.65 (m, 1H), 7.51-7.40 (m, 3H, 1H D₂O exchangeable), 7.35-7.29 (m, 2H), 7.28-7.24 (m, 1H), 7.24-7.11 (m, 2H), 6.92-6.84 (m, 1H), 5.16 (s, 2H), 3.88 (s, 2H), 3.43 (s, 3H); MS (ES+): 416.10 (M+1); (ES−): 414.10 (M−1).

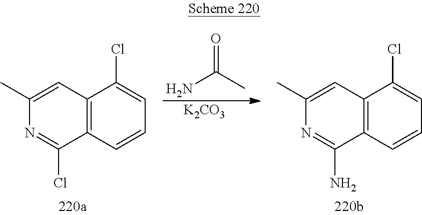

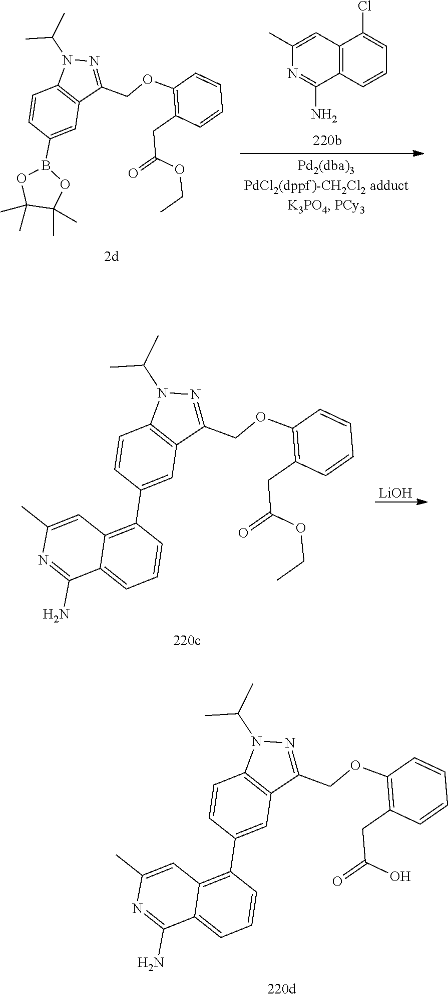

### Preparation of 2-(2-((5-(1-amino-3-methylisoquinolin-5-yl)-1-isopropyl-1H-indazol-3-yl)methoxy)phenyl)acetic acid (220d)

### Step-1: Preparation of 5-chloro-3-methylisoquinolin-1-amine (220b)

Compound 220b was prepared according to the procedure reported in step-1 of scheme 74 from 1,5-dichloro-3-methylisoquinoline (220a)(500 mg, 2.358 mmol; CAS #1206973-83-2) using acetamide (2785 mg, 47.2 mmol) and K₂CO₃ (978 mg, 7.07 mmol) to afford after workup and purification using method-E, 5-chloro-3-methylisoquinolin-1-amine (220b) (230 mg, 50.6% yield) as a white solid; ¹H NMR (300 MHz, DMSO-d₆) δ 8.14 (dt, J=8.4, 1.0 Hz, 1H), 7.72 (dd, J=7.6, 1.0 Hz, 1H), 7.34 (dd, J=8.4, 7.5 Hz, 1H), 7.00 (s, 2H), 6.93 (t, J=0.9 Hz, 1H), 2.37 (d, J=0.7 Hz, 3H); MS (ES+): 193.05 & 195.05 (M+1).

### Step-2: Preparation of ethyl 2-(2-((5-(1-amino-3-methylisoquinolin-5-yl)-1-isopropyl-1H-indazol-3-yl)methoxy)phenyl)acetate (220c)

Compound 220c was prepared according to the procedure reported in step-5 of scheme 1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (200 mg, 0.418 mmol) in dioxane/THF (4 mL, 1:1) using 5-chloro-3-methylisoquinolin-1-amine (220b) (161 mg, 0.836 mmol), K₃PO₄ (2 M aqueous solution, 0.836 mL, 1.672 mmol), PCy₃ (23.45 mg, 0.084 mmol), PdCl₂(dppf)-CH₂Cl₂ adduct (34.3 mg, 0.042 mmol) and Pd₂(dba)₃ (38.3 mg, 0.042 mmol) to afford after workup and purification using method-P, ethyl 2-(2-((5-(1-amino-3-methylisoquinolin-5-yl)-1-isopropyl-1H-indazol-3-yl)methoxy)phenyl)acetate (220c) (150 mg, 70.5% yield) as a clear oil; MS (ES+): 509.30 (M+1).

### Step-3: Preparation of 2-(2-((5-(1-amino-3-methylisoquinolin-5-yl)-1-isopropyl-1H-indazol-3-yl)methoxy)phenyl)acetic acid (220d)

Compound 220d was prepared according to the procedure reported in step-2 of scheme 1 from ethyl 2-(2-((5-(1-amino-3-methylisoquinolin-5-yl)-1-isopropyl-1H-indazol-3-yl)methoxy)phenyl)acetate (220c) (150 mg, 0.295 mmol) in MeOH/THF (6 mL, 1:1) using a solution of LiOH·H₂O (74.3 mg, 1.770 mmol) in water (2 mL) to afford after workup and purification using method-G, 2-(2-((5-(1-amino-3-methylisoquinolin-5-yl)-1-isopropyl-1H-indazol-3-yl)methoxy)phenyl)acetic acid (220d) (15 mg, 10.58% yield) free base as a white solid; ¹H NMR (300 MHz, DMSO-d₆) δ 8.27 (d, J=8.2 Hz, 1H), 7.87-7.77 (m, 2H), 7.67-7.58 (m, 1H), 7.56-7.34 (m, 4H, 2H D₂O exchangeable), 7.30-7.21 (m, 2H), 7.21-7.11 (m, 1H), 6.95-6.82 (m, 1H), 6.66 (s, 1H), 5.44 (s, 2H), 5.14-4.95 (m, 1H), 3.48 (s, 2H), 2.27 (s, 3H), 1.55 (d, J=6.5 Hz, 6H); MS (ES+): 481.20 (M+1); (ES−): 479.20 (M−1).

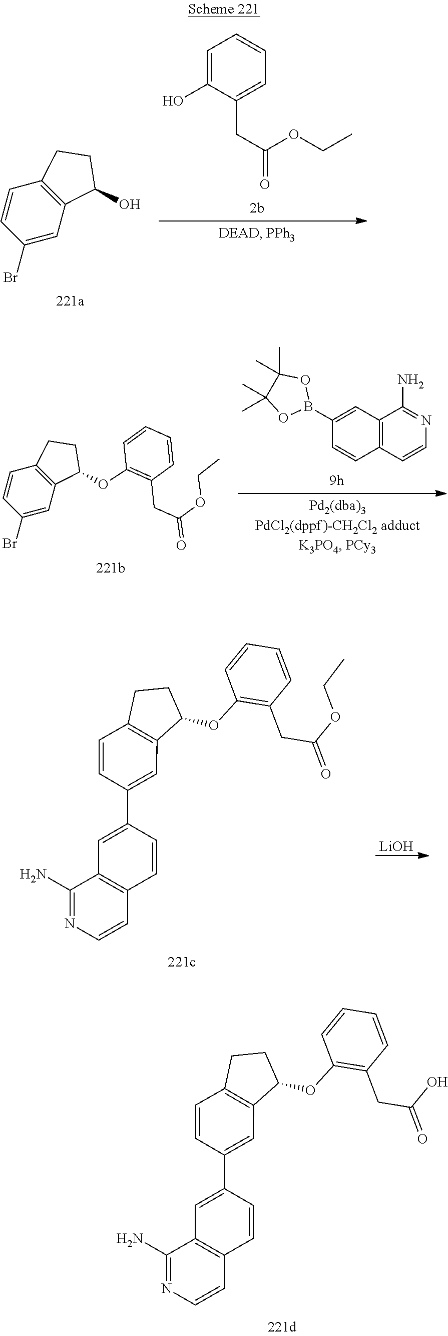

### Preparation of (S)-2-(2-((6-(1-aminoisoquinolin-7-yl)-2,3-dihydro-1H-inden-1-yl)oxy)phenyl)acetic acid (221d)

### Step-1: Preparation of (S)-ethyl 2-(2-((6-bromo-2,3-dihydro-1H-inden-1-yl)oxy)phenyl)acetate (221b)

Compound 221b was prepared according to the procedure reported in step-1 of scheme 8 from (R)-6-bromo-2,3-dihydro-1H-inden-1-ol (221a) (500 mg, 2.347 mmol; CAS #1270291-42-3) in DCM (10 mL) using ethyl 2-(2-hydroxyphenyl)acetate (2b) (846 mg, 4.69 mmol), PPh₃ (1231 mg, 4.69 mmol) and a solution of DEAD (817 mg, 4.69 mmol) in DCM to afford after workup and purification using method-L, (S)-ethyl 2-(2-((6-bromo-2,3-dihydro-1H-inden-1-yl)oxy)phenyl)acetate (221b) (310 mg, 35.2% yield) as a yellow oil; ¹H NMR (300 MHz, DMSO-d₆) δ 7.52-7.42 (m, 2H), 7.35-7.25 (m, 2H), 7.25-7.15 (m, 2H), 6.96-6.88 (m, 1H), 5.88-5.77 (m, 1H), 4.01-3.87 (m, 2H), 3.49 (s, 2H), 3.05-2.91 (m, 1H), 2.91-2.76 (m, 1H), 2.69-2.56 (m, 1H), 2.03-1.89 (m, 1H), 1.04 (t, J=7.1 Hz, 3H); MS (ES+): 397.00 & 399.00 (M+Na):

### Step-2: Preparation of (S)-ethyl 2-(2-((6-(1-aminoisoquinolin-7-yl)-2,3-dihydro-1H-inden-1-yloxy)phenyl)acetate (221c)

Compound 221c was prepared according to the procedure reported in step-5 of scheme 1 from (S)-ethyl 2-(2-((6-bromo-2,3-dihydro-1H-inden-1-yl)oxy)phenyl)acetate (221b) (200 mg, 0.533 mmol) in dioxane/THF (4 mL, 1:1) using 7-(4,4,5,5-tetramethyl-1,3,2-dioxaborolan-2-yl)isoquinolin-1-amine (9h) (216 mg, 0.799 mmol), K₃PO₄ (2 M aqueous solution, 1.066 mL, 2.132 mmol), PCy₃ (29.9 mg, 0.107 mmol), PdCl₂(dppf)-CH₂Cl₂ adduct (43.5 mg, 0.053 mmol) and Pd₂(dba)₃ (48.8 mg, 0.053 mmol) to afford after workup and purification using method-Y, (S)-ethyl 2-(2-((6-(1-aminoisoquinolin-7-yl)-2,3-dihydro-1H-inden-1-yl)oxy)phenyl)acetate (221c) (150 mg, 64.2% yield) as a clear oil; ¹H NMR (300 MHz, DMSO-d₆) δ 8.51 (s, 1H), 7.96-7.90 (m, 1H), 7.86-7.74 (m, 4H), 7.46 (d, J=7.9 Hz, 1H), 7.34-7.18 (m, 3H), 7.00-6.88 (m, 4H, 2H D₂O exchangeable), 5.92 (t, J=5.6 Hz, 1H), 3.78 (q, J=7.1 Hz, 2H), 3.50 (s, 2H), 3.15-3.02 (m, 1H), 3.02-2.84 (m, 1H), 2.76-2.61 (m, 1H), 2.07-1.93 (m, 1H), 0.79 (t, J=7.1 Hz, 3H); MS (ES+): 439.20 (M+1).

### Step-3: Preparation of (S)-2-(2-((6-(1-aminoisoquinolin-7-yl)-2,3-dihydro-1H-inden-1-yl)oxy)phenyl)acetic acid (221d)

Compound 221d was prepared according to the procedure reported step-2 of scheme 1 from (S)-ethyl 2-(2-((6-(1-aminoisoquinolin-7-yl)-2,3-dihydro-1H-inden-1-yl)oxy)phenyl)acetate (221c) (200 mg, 0.456 mmol) in MeOH/THF (6 mL, 1:1) using a solution of LiOH·H₂O (115 mg, 2.74 mmol) in water (2 mL) to afford after workup and purification using method-G, (S)-2-(2-((6-(1-aminoisoquinolin-7-yl)-2,3-dihydro-1H-inden-1-yl)oxy)phenyl)acetic acid (221d) (30 mg, 16.03% yield) HCl salt as a white solid; ¹H NMR (300 MHz, DMSO-d₆) δ 12.23 (s. 1H, D₂O exchangeable), 8.57-8.45 (m, 1H), 8.01-7.92 (m, 1H), 7.86-7.74 (m, 4H), 7.49-7.42 (m, 1H), 7.33-7.25 (m, 2H), 7.25-7.17 (m, 1H), 7.10 (s, 2H, D₂O exchangeable), 6.98-6.87 (m, 2H), 5.90 (t, J=5.7 Hz, 1H), 3.47 (s, 2H), 3.16-3.03 (m, 1H), 3.01-2.87 (m, 1H), 2.76-2.60 (m, 1H), 2.11-1.96 (m, 1H); MS (ES+): 411.15 (M+1); (ES−): 409.10 (M−1).

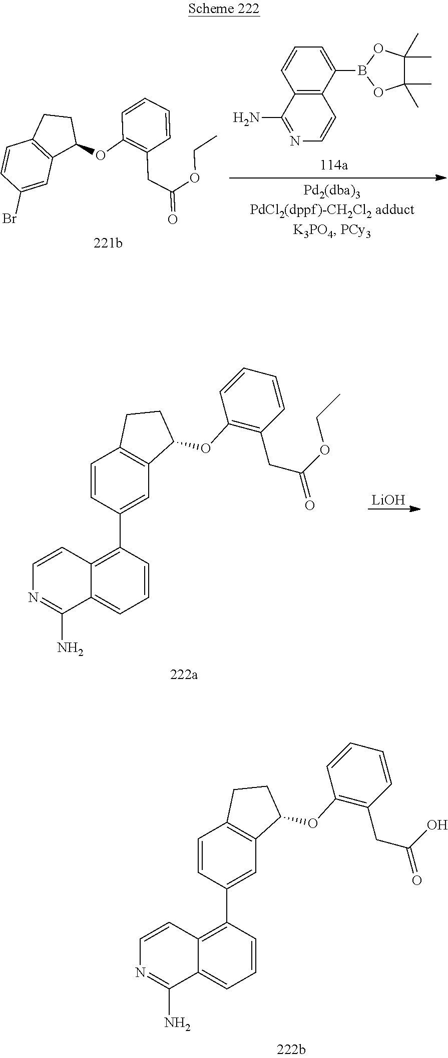

### Preparation of (S)-2-(2-((6-(1-aminoisoquinolin-5-yl)-2,3-dihydro-1H-inden-1-yl)oxy)phenyl)acetic acid (222b)

### Step-1: Preparation of (S)-ethyl 2-(2-((6-(1-aminoisoquinolin-5-yl)-2,3-dihydro-1H-inden-1-yl)oxy)phenyl)acetate (222a)

Compound 222a was prepared according to the procedure reported in step-5 of scheme 1 from (S)-ethyl 2-(2-((6-bromo-2,3-dihydro-1H-inden-1-yl)oxy)phenyl)acetate (221b) (200 mg, 0.533 mmol) in dioxane/THF (4 mL, 1:1) using 5-(4,4,5,5-tetramethyl-1,3,2-dioxaborolan-2-yl)isoquinolin-1-amine (114a) (216 mg, 0.799 mmol), K₃PO₄ (2 M aqueous solution, 1.066 mL, 2.132 mmol), PCy₃ (29.9 mg, 0.107 mmol), PdCl₂(dppf)-CH₂Cl₂ adduct (43.5 mg, 0.053 mmol) and Pd₂(dba)₃ (48.8 mg, 0.053 mmol) to afford after workup and purification using method-Y, (S)-ethyl 2-(2-((6-(1-aminoisoquinolin-5-yl)-2,3-dihydro-1H-inden-1-yl)oxy)phenyl)acetate (222a) (150 mg, 64.2% yield) as a clear oil; ¹H NMR (300 MHz, DMSO-d₆) δ 8.27-8.15 (m, 1H), 7.73 (d, J=6.1 Hz, 1H), 7.54-7.42 (m, 3H), 7.43-7.31 (m, 2H), 7.31-7.16 (m, 3H), 6.95-6.81 (m, 3H, 2H D₂O exchangeable), 6.81-6.73 (m, 1H), 5.91 (t, J=5.8 Hz, 1H), 3.75-3.57 (m, 2H), 3.47 (d, J=5.7 Hz, 2H), 3.19-3.05 (m, 1H), 3.05-2.89 (m, 1H), 2.80-2.63 (m, 1H), 2.12-1.95 (m, 1H), 0.78 (t, J=7.1 Hz, 3H); MS (ES+): 439.20 (M+1).

### Step-2: Preparation of (S)-2-(2-((6-(1-aminoisoquinolin-5-yl)-2,3-dihydro-1H-inden-1-yl)oxy)phenyl)acetic acid (222b)

Compound 222b was prepared according to the procedure reported step-2 of scheme 1 from (S)-ethyl 2-(2-((6-(1-aminoisoquinolin-5-yl)-2,3-dihydro-1H-inden-1-yl)oxy)phenyl)acetate (222a) (200 mg, 0.456 mmol) in MeOH/THF (6 mL, 1:1) using a solution of LiOH·H₂O (115 mg, 2.74 mmol) in water (2 mL) to afford after workup and purification using method-G, (S)-2-(2-((6-(1-aminoisoquinolin-5-yl)-2,3-dihydro-1H-inden-1-yl)oxy)phenyl)acetic acid (222b) (65 mg, 34.7% yield) HCl salt as a white solid; ¹H NMR (300 MHz, DMSO-d₆) δ 12.05 (s, 1H, D₂O exchangeable), 8.27-8.18 (m, 1H), 7.72 (d, J=6.2 Hz, 1H), 7.59-7.51 (m, 2H), 7.49-7.43 (m, 1H), 7.42-7.33 (m, 2H), 7.29-7.22 (m, 2H), 7.22-7.14 (m, 1H), 7.07 (s, 2H, D₂O) exchangeable), 6.93-6.86 (m, 1H), 6.78 (d, J=6.2 Hz, 1H), 5.90 (t, J=5.8 Hz, 1H), 3.43 (s, 2H), 3.20-3.05 (m, 1H), 3.04-2.89 (m, 1H), 2.78-2.62 (m, 1H), 2.15-1.98 (m, 1H); MS (ES+): 411.10 (M+1); (ES−): 409.10 (M−1).

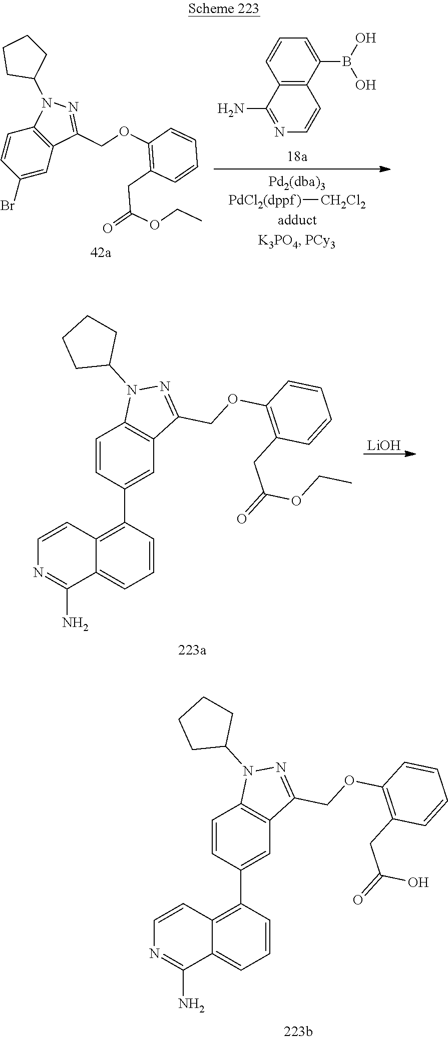

### Preparation of 2-(2-((5-(1-aminoisoquinolin-5-yl)-1-cyclopentyl-1H-indazol-3-yl)methoxy)phenyl)acetic acid (223b)

### Step-1: Preparation of ethyl 2-(2-((5-(1-aminoisoquinolin-5-yl)-1-cyclopentyl-1H-indazol-3-yl)methoxy)phenyl)acetate (223a)

Compound 223a was prepared according to the procedure reported in step-5 of scheme 1 from ethyl 2-(2-((5-bromo-1-cyclopentyl-1H-indazol-3-yl)methoxy)phenyl)acetate (42a) (200 mg, 0.437 mmol) in dioxane/THF (4 mL, 1:1) using (I-aminoisoquinolin-5-yl)boronic acid (18a) (123 mg, 0.656 mmol), K₃PO₄ (2 M aqueous solution, 0.875 mL, 1.749 mmol), PCy₃ (24.53 mg, 0.087 mmol), PdCl₂(dppf)-CH₂Cl₂ adduct (35.7 mg, 0.044 mmol) and Pd₂(dba)₃ (40.0 mg, 0.044 mmol) to afford after workup and purification using method-Y, ethyl 2-(2-((5-(1-aminoisoquinolin-5-yl)-1-cyclopentyl-1H-indazol-3-yl)methoxy)phenyl)acetate (223a) (150 mg, 65.9% yield) as a clear oil; ¹H NMR (300 MHz, DMSO-d₆) ¿ 8.22 (d, J=8.1 Hz, 1H), 7.88-7.70 (m, 3H), 7.61-7.46 (m, 3H), 7.25 (d, J=4.2 Hz, 2H), 7.15 (d, J=7.4 Hz, 1H), 6.94-6.85 (m, 3H), 6.82 (d, J=6.1 Hz, 1H), 5.42 (s, 2H), 5.23 (p. J=7.2 Hz, 1H), 3.60-3.52 (m, 2H), 3.50 (s, 2H), 2.23-2.13 (m, 2H), 2.13-2.03 (m, 2H), 1.96-1.87 (m, 2H), 1.68-1.60 (m, 2H), 0.70 (t, J=7.1 Hz, 3H); MS (ES+): 521.25 (M+1).

### Step-2: Preparation of 2-(2-((5-(1-aminoisoquinolin-5-yl)-1-cyclopentyl-1H-indazol-3-yl)methoxy)phenyl)acetic acid (223b)

Compound 223b was prepared according to the procedure reported step-2 of scheme 1 from ethyl 2-(2-((5-(1-aminoisoquinolin-5-yl)-1-cyclopentyl-1H-indazol-3-yl)methoxy)phenyl)acetate (223a) (150 mg, 0.288 mmol) in MeOH/THF (6 mL, 1:1) using a solution of LiOH·H₂O (72.5 mg, 1.729 mmol) in water (2 mL) to afford after workup and purification using method-G, 2-(2-((5-(1-aminoisoquinolin-5-yl)-1-cyclopentyl-1H-indazol-3-yl)methoxy)phenyl)acetic acid (223b) (45 mg, 31.7% yield) HCl salt as a white solid; ¹H NMR (300 MHz, DMSO-d₆) δ 13.30 (s, 1H, D₂O exchangeable), 12.10 (s, 1H, D₂O exchangeable), 9.15 (s, 2H, D₂O exchangeable), 8.62 (d, J=8.3 Hz, 1H), 7.95 (d, J=7.2 Hz, 1H), 7.92-7.78 (m, 3H), 7.62 (d, J=7.2 Hz, 1H), 7.51-7.42 (m, 1H), 7.31-7.20 (m, 2H), 7.20-7.13 (m, 1H), 6.99 (d, J=7.2 Hz, 1H), 6.95-6.83 (m, 1H), 5.44 (s, 2H), 5.32-5.13 (m, 1H), 3.47 (s, 2H), 2.25-2.11 (m, 2H), 2.11-1.99 (m, 2H), 1.99-1.83 (m, 2H), 1.82-1.62 (m, 2H); MS (ES+): 493.20 (M+1); (ES−): 491.20 (M−1); Analysis calculated for C₃₀H₂₈N₄O₃·HCl·1.75H₂O: C, 64.28; H, 5.84; Cl, 6.32; N, 9.99; Found: C, 64.22; H, 5.72; Cl, 6.09; N, 9.94.

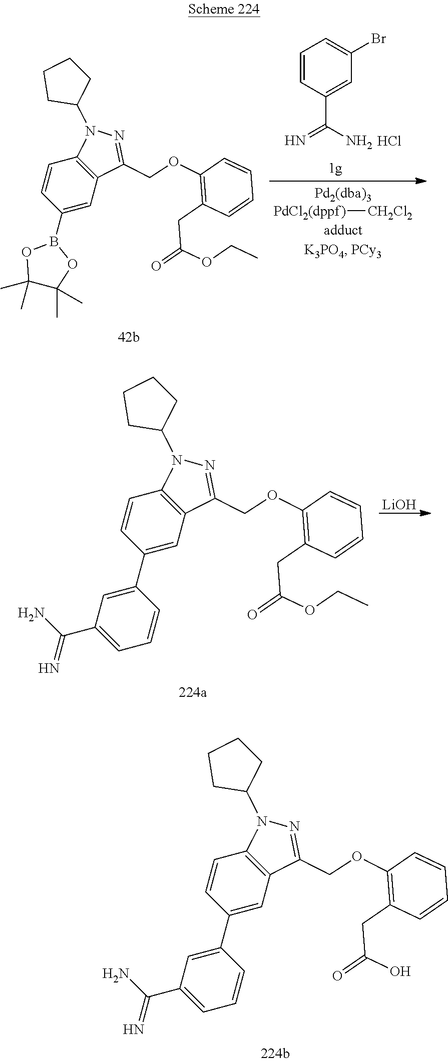

### Preparation of 2-(2-((5-(3-carbamimidoylphenyl)-1-cyclopentyl-1H-indazol-3-yl)methoxy)phenyl)acetic acid (224b)

### Step-1: Preparation of ethyl 2-(2-((5-(3-carbamimidoylphenyl)-1-cyclopentyl-1H-indazol-3-yl)methoxy)phenyl)acetate (224a)

Compound 224a was prepared according to the procedure reported in step-5 of scheme 1 from ethyl 2-(2-((1-cyclopentyl-5-(4,4,5,5-tetramethyl-1,3,2-dioxaborolan-2-yl)-1H-indazol-3-yl)methoxy)phenyl)acetate (42b) (500 mg, 0.991 mmol) in dioxane/THF (6 mL, 1:1) using 3-bromobenzimidamide hydrochloride (1g) (467 mg, 1.982 mmol), K₃PO₄ (2 M aqueous solution, 1.982 mL, 3.96 mmol), PCy₃ (55.6 mg, 0.198 mmol), PdCl₂(dppf)-CH₂Cl₂ adduct (81 mg, 0.099 mmol) and Pd₂(dba)₃ (91 mg, 0.099 mmol) to afford after workup and purification using method-AI, ethyl 2-(2-((5-(3-carbamimidoylphenyl)-1-cyclopentyl-1H-indazol-3-yl)methoxy)phenyl)acetate (224a) (300 mg, 60.9% yield) as a clear oil; ¹H NMR (300 MHz, DMSO-d₆) δ 9.39 (s, 3H), 8.22-8.05 (m, 3H), 7.91-7.84 (m, 2H), 7.81-7.74 (m, 1H), 7.74-7.65 (m, 1H), 7.34-7.25 (m, 2H), 7.19 (d, J=7.4 Hz, 1H), 6.97-6.88 (m, 1H), 5.43 (s, 2H), 5.23 (p, J=7.2 Hz, 1H), 3.66 (q, J=7.1 Hz, 2H), 3.53 (s, 2H), 2.21-2.09 (m, 2H), 2.09-1.98 (m, 2H), 1.94-1.85 (m, 2H), 1.76-1.69 (m, 2H), 0.78 (t, J=7.1 Hz, 3H); MS (ES+): 497.20 (M+1).

### Step-2: Preparation of 2-(2-((5-(3-carbamimidoylphenyl)-1-cyclopentyl-1H-indazol-3-yl)methoxy)phenyl)acetic acid (224b)

Compound 224b was prepared according to the procedure reported step-2 of scheme 1 from ethyl 2-(2-((5-(3-carbamimidoylphenyl)-1-cyclopentyl-1H-indazol-3-yl)methoxy)phenyl)acetate (224a) (300 mg, 0.604 mmol) in MeOH/THF (6 mL, 1:1) using a solution of LiOH·H₂O (152 mg, 3.62 mmol in water (2 mL) to afford after workup and purification using method-G, 2-(2-((5-(3-carbamimidoylphenyl)-1-cyclopentyl-1H-indazol-3-yl)methoxy)phenyl)acetic acid (224b) (55 mg, 19.43% yield) HCl salt as a white solid; ¹H NMR (300 MHz, DMSO-d₆) δ 12.09 (s, 1H, D₂O exchangeable), 9.48 (s, 2H, D₂O exchangeable), 9.23 (s, 2H, D₂O exchangeable), 8.18 (d, J=9.0 Hz, 2H), 8.09 (d, J=7.7 Hz, 1H), 7.92-7.82 (m, 2H), 7.82-7.74 (m, 1H), 7.70 (t, J=7.7 Hz, 1H), 7.33-7.23 (m, 2H), 7.23-7.14 (m, 1H), 6.99-6.83 (m, 1H), 5.46 (s, 2H), 5.30-5.10 (m, 1H), 3.52 (s, 2H), 2.26-2.10 (m, 2H), 2.10-1.97 (m, 2H), 1.97-1.82 (m, 2H), 1.80-1.63 (m, 2H); MS (ES+): 469.20 (M+1); (ES−): 467.20 (M−1).

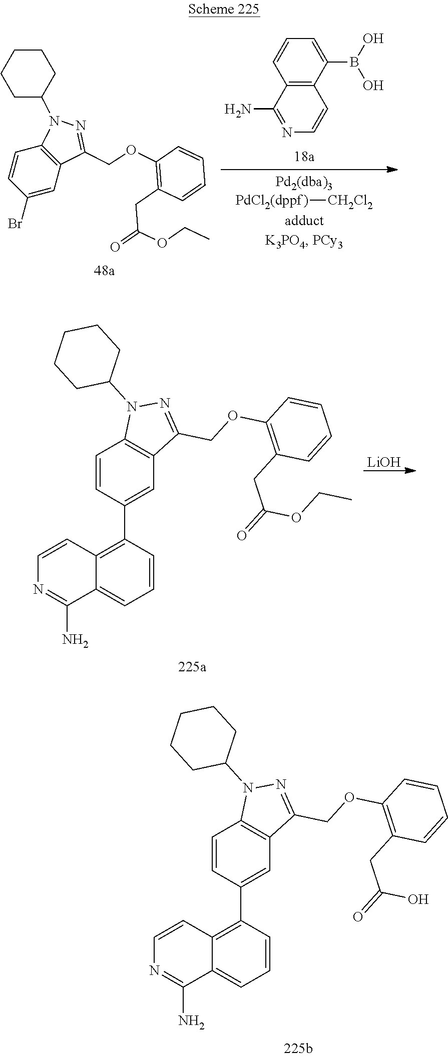

### Preparation of 2-(2-((5-(1-aminoisoquinolin-5-yl)-1-cyclohexyl-1H-indazol-3-yl)methoxy)phenyl)acetic acid (225b)

### Step-1: Preparation of ethyl 2-(2-((5-(1-aminoisoquinolin-5-yl)-1-cyclohexyl-1H-indazol-3-yl)methoxy)phenyl)acetate (225a)

Compound 225a was prepared according to the procedure reported in step-5 of scheme 1 from ethyl 2-(2-((5-bromo-1-cyclohexyl-1H-indazol-3-yl)methoxy)phenyl)acetate (48a) (300 mg, 0.636 mmol) in dioxane/THF (4 mL, 1:1) using (I-aminoisoquinolin-5-yl)boronic acid (18a) (179 mg, 0.955 mmol), K₃PO₄ (2 M aqueous solution, 1.273 mL, 2.55 mmol), PCy₃ (35.7 mg, 0.127 mmol), PdCl₂(dppf)-CH₂Cl₂ adduct (52.0 mg, 0.064 mmol) and Pd₂(dba)₃ (58.3 mg, 0.064 mmol) to afford after workup and purification using method-Y, ethyl 2-(2-((5-(1-aminoisoquinolin-5-yl)-1-cyclohexyl-1H-indazol-3-yl)methoxy)phenyl)acetate (225a) (250 mg, 73.5% yield) as a clear oil; ¹H NMR (300 MHz, DMSO-d₆) δ 8.22 (d, J=8.1 Hz, 1H), 7.86 (d, J=8.8 Hz, 1H), 7.78 (s, 1H), 7.74 (d, J=6.1 Hz, 1H), 7.62-7.44 (m, 3H), 7.28-7.24 (m, 2H), 7.16 (d, J=7.6 Hz, 1H), 6.96-6.86 (m, 3H), 6.83 (d, J=6.2 Hz, 1H), 5.41 (s, 2H), 4.78-4.59 (m, 1H), 3.58-3.46 (m, 4H), 2.04-1.68 (m, 10H), 0.69 (t, J=7.1 Hz, 3H); MS (ES+): 535.30 (M+1).

### Step-2: Preparation of 2-(2-((5-(1-aminoisoquinolin-5-yl)˜1-cyclohexyl-1H-indazol-3-yl)methoxy)phenyl)acetic acid (225b)

Compound 225b was prepared according to the procedure reported step-2 of scheme 1 from ethyl 2-(2-((5-(1-aminoisoquinolin-5-yl)-1-cyclohexyl-1H-indazol-3-yl)methoxy)phenyl)acetate (225a) (250 mg, 0.468 mmol) in MeOH/THF (6 mL, 1:1) using a solution of LiOH·H₂O (118 mg, 2.81 mmol) in water (2 mL) to afford after workup and purification using method-G, 2-(2-((5-(1-aminoisoquinolin-5-yl)-1-cyclohexyl-1H-indazol-3-yl)methoxy)phenyl)acetic acid (225b) (75 mg, 31.7% yield) HCl salt as a white solid; ¹H NMR (300 MHz, DMSO-d₆) δ 13.29 (s, 1H, D₂O exchangeable), 12.03 (s, 1H, D₂O exchangeable), 9.14 (s, 2H, D₂O exchangeable), 8.61 (d, J=8.3 Hz, 1H), 8.00-7.89 (m, 2H), 7.89-7.80 (m, 2H), 7.62 (d, J=7.2 Hz, 1H), 7.45 (dd, J=8.7, 1.6 Hz, 1H), 7.28-7.20 (m, 2H), 7.20-7.13 (m, 1H), 7.00 (d, J=7.3 Hz, 1H), 6.94-6.85 (m, 1H), 5.44 (s, 2H), 4.78-4.58 (m, 1H), 3.48 (s, 2H), 2.07-1.81 (m, 6H), 1.81-1.64 (m, 1H), 1.64-1.43 (m, 2H), 1.43-1.19 (m, 1H); MS (ES+): 507.20 (M+1); (ES−): 505.20 (M−1).

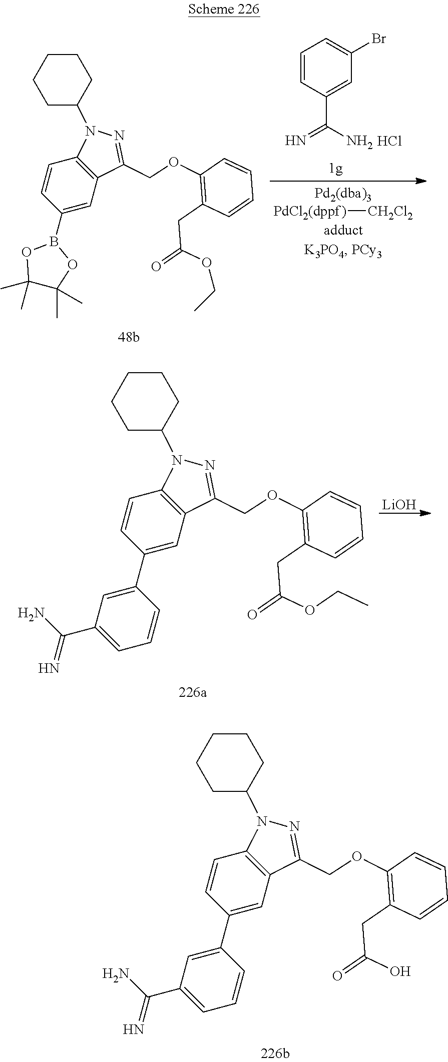

### Preparation of 2-(2-((5-(3-carbamimidoylphenyl)-1-cyclohexyl-1H-indazol-3-yl)methoxy)phenyl)acetic acid (226b)

### Step-1: Preparation of ethyl 2-(2-((5-(3-carbamimidoylphenyl)-1-cyclohexyl-1H-indazol-3-yl)methoxy)phenyl)acetate (226a)

Compound 226a was prepared according to the procedure reported in step-5 of scheme-1 from ethyl 2-(2-((1-cyclohexyl-5-(4,4,5,5-tetramethyl-1,3,2-dioxaborolan-2-yl)-1H-indazol-3-yl)methoxy)phenyl)acetate (48b) (430 mg, 0.829 mmol) in dioxane/THF (6 mL, 1:1) using 3-bromobenzimidamide hydrochloride (1g) (391 mg, 1.659 mmol), K₃PO₄ (2 M aqueous solution, 1.659 mL, 3.32 mmol), PCy₃ (46.5 mg, 0.166 mmol), PdCl₂(dppf)-CH₂Cl₂ adduct (67.7 mg, 0.083 mmol) and Pd₂(dba)₃ (76 mg, 0.083 mmol) to afford after workup and purification using method-AI, ethyl 2-(2-((5-(3-carbamimidoylphenyl)-1-cyclohexyl-1H-indazol-3-yl)methoxy)phenyl)acetate (226a)(160 mg, 37.8% yield) as a clear oil; 3H NMR (300 MHz, DMSO-d₆) δ 9.24 (s, 3H), 8.19-8.01 (m, 3H), 7.92-7.81 (m, 2H), 7.81-7.73 (m, 1H), 7.73-7.64 (m, 1H), 7.34-7.24 (m, 2H), 7.19 (d, J=7.3 Hz, 1H), 6.98-6.86 (m, 1H), 5.43 (s, 2H), 4.76-4.58 (m, 1H), 3.70-3.62 (m, 2H), 3.54 (s, 2H), 1.96-1.69 (m, 10H), 0.77 (t, J=7.1 Hz, 3H); MS (ES+): 511.30 (M+1).

### Step-2: Preparation of 2-(2-((5-(3-carbamimidoylphenyl)-1-cyclohexyl-1H-indazol-3-yl)methoxy)phenyl)acetic acid (226b)

Compound 226b was prepared according to the procedure reported step-2 of scheme-1 from ethyl 2-(2-((5-(3-carbamimidoylphenyl)-1-cyclohexyl-1H-indazol-3-yl)methoxy)phenyl)acetate (226a) (160 mg, 0.313 mmol) in MeOH/THF (6 mL, 1:1) using a solution of LiOH·H₂O (79 mg, 1.880 mmol) in water (2 mL) to afford after workup and purification using method-G, 2-(2-((5-(3-carbamimidoylphenyl)-1-cyclohexyl-1H-indazol-3-yl)methoxy)phenyl)acetic acid (226b) (60 mg, 39.7% yield) HCl salt as a white solid; ¹H NMR (300 MHz, DMSO-d₆) δ 12.12 (s, 1H, D₂O exchangeable), 9.54 (s, 2H, D₂O exchangeable), 9.33 (s, 2H, D₂O exchangeable), 8.23-8.14 (m, 2H), 8.09 (dt, 1H), 7.89 (d, 2H), 7.83-7.75 (m, 1H), 7.69 (t, J=7.8 Hz, 1H), 7.32-7.23 (m, 2H), 7.23-7.13 (m, 1H), 6.98-6.84 (m, 1H), 5.45 (s, 2H), 4.77-4.55 (m, 1H), 3.54 (s, 2H), 2.05-1.79 (m, 6H), 1.79-1.16 (m, 4H); MS (ES+): 483.20 (M+1); (ES−): 481.20 (M−1).

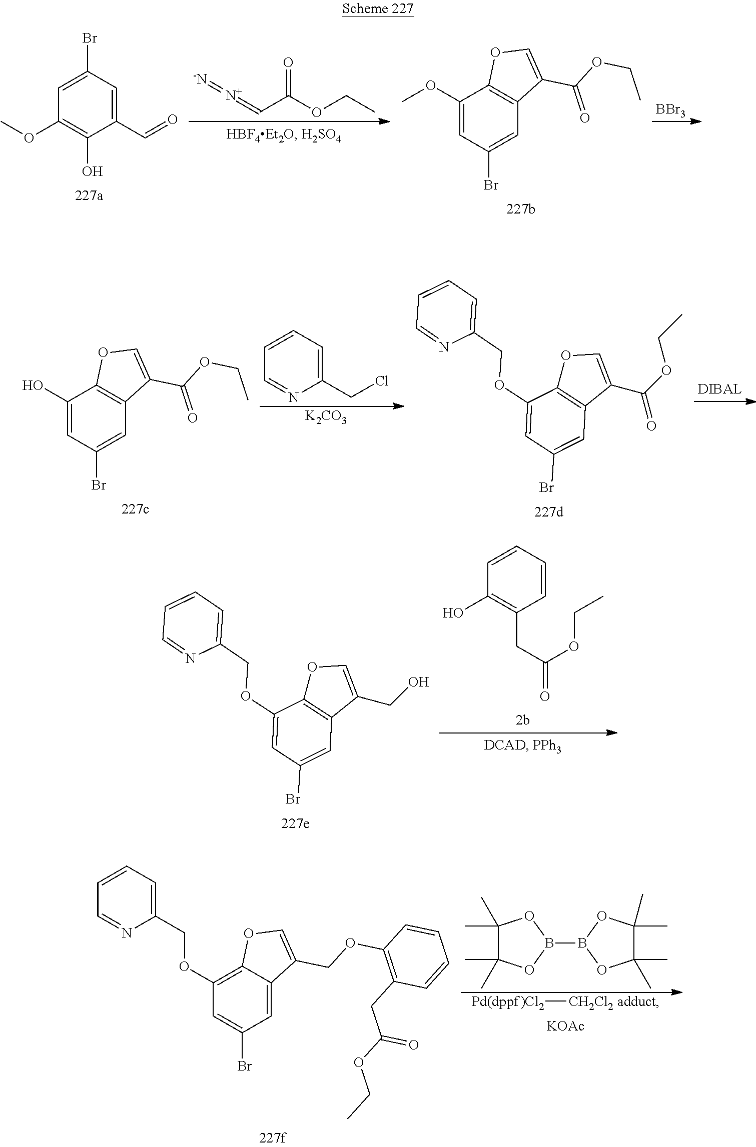

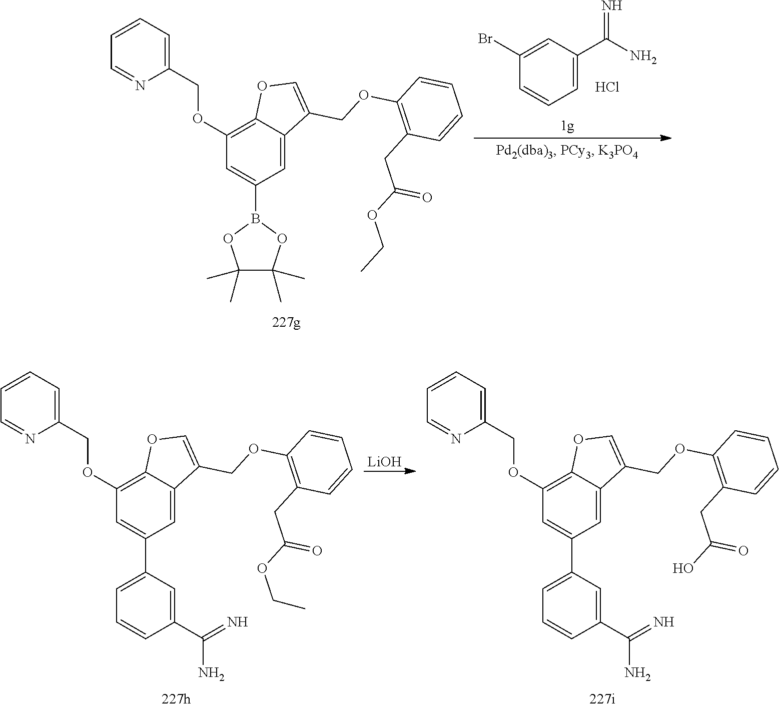

### Preparation of 2-(2-((5-(3-carbamimidoylphenyl)-7-(pyridin-2-ylmethoxy)benzofuran-3-yl)methoxy)phenyl)acetic acid (227i)

### Step-1: Preparation of ethyl 5-bromo-7-methoxybenzofuran-3-carboxylate (227b)

To a solution of 5-bromo-2-hydroxy-3-methoxybenzaldehyde (227a) (10 g, 43.3 mmol; CAS #5034-74-2) in DCM (50 mL) was added HBF₄·Et₂O (1.071 mL, 4.33 mmol), followed by a solution of ethyl diazoacetate (15% in toluene, 62.9 mL, 91 mmol) at RT (gas evolved after addition). The resulting mixture was stirred for 20 min at RT and was concentrated to dryness to afford a brown thick oil. To the resulting oil was added concentrated sulfuric acid (5.77 mL, 108 mmol) slowly and was stirred for 10 min at RT. The mixture was diluted with DCM (100 mL) and NaHCO₃ (20 g) was added in portions. The mixture was stirred for 20 h at RT, filtered through a pad of Celite and the filtrate was concentrated in vacuum. The residue obtained was purified using method-AO to afford ethyl 5-bromo-7-methoxybenzofuran-3-carboxylate (227b) (5.9 g, 45.6% yield) as a white solid; ¹H NMR (300 MHz, DMSO-d₆) δ 8.79 (s, 1H), 7.65 (d, J=1.7 Hz, 1H), 7.24 (d, J=1.8 Hz, 1H), 4.35 (q, J=7.1 Hz, 2H), 3.98 (s, 3H), 1.34 (t, J=7.1 Hz, 3H).

### Step-2: Preparation of ethyl 5-bromo-7-hydroxybenzofuran-3-carboxylate (227c)

Compound 227c was prepared according to the procedure reported in step-1 of scheme-94 from ethyl 5-bromo-7-methoxybenzofuran-3-carboxylate (227b) (2 g, 6.69 mmol) in DCM (50 mL) using boron tribromide (1.454 mL, 15.38 mmol) to afford after workup and purification using method-K, ethyl 5-bromo-7-hydroxybenzofuran-3-carboxylate (227c) (1.25 g, 66% yield) as a colorless oil; ¹H NMR (300 MHz, DMSO-d₆) δ 10.95 (s, 1H), 8.76 (s, 1H), 7.51 (d, J=1.9 Hz, 1H), 6.99 (d, J=1.9 Hz, 1H), 4.34 (q, J=7.1 Hz, 2H), 1.34 (t, J=7.1 Hz, 3H).

### Step-3: Preparation of ethyl 5-bromo-7-(pyridin-2-ylmethoxy)benzofuran-3-carboxylate (227d)

Compound 227d was prepared according to the procedure reported in step-3 of scheme-106 from ethyl 5-bromo-7-hydroxybenzofuran-3-carboxylate (227c) (450 mg, 1.578 mmol) in DMF (5 mL) using 2-(chloromethyl)pyridine. HCl (259 mg, 1.578 mmol), K₂CO₃ (654 mg, 4.74 mmol) and stirring at rt for 14 h. This gave after workup ethyl 5-bromo-7-(pyridin-2-ylmethoxy)benzofuran-3-carboxylate (227d) (568 mg, 96% yield) as an off-white solid; ¹H NMR (300 MHz, DMSO-d₆) δ 8.82 (s, 1H), 8.62 (ddd, J=4.9, 1.8, 0.9 Hz, 1H), 7.89 (td, J=7.7, 1.8 Hz, 1H), 7.68 (d, J=1.7 Hz, 1H), 7.60 (d, J=8.0 Hz, 1H), 7.45-7.34 (m, 2H), 5.42 (s, 2H), 4.36 (q, J=7.1 Hz, 2H), 1.35 (t, J=7.1 Hz, 3H); MS (ES+): 376.0 (M+1).

### Step-4: Preparation of (5-bromo-7-(pyridin-2-ylmethoxy)benzofuran-3-yl)methanol (227e)

Compound 227e was prepared according to the procedure reported in step-1 of scheme-2 from ethyl 5-bromo-7-(pyridin-2-ylmethoxy)benzofuran-3-carboxylate (227d) (500 mg, 1.329 mmol) in DCM (2.66 mL), using 1.0 M solution of DIBAL in DCM (3.99 mL, 3.99 mmol) to afford after workup and purification using method-BC, (5-bromo-7-(pyridin-2-ylmethoxy)benzofuran-3-yl)methanol (227e) (0.141 g, 32% yield); ¹H NMR (300 MHz. DMSO-d₆) δ 8.63-8.59 (m, 1H), 7.92 (s, 1H), 7.88 (td, J=7.7, 1.8 Hz, 1H), 7.58 (d, J=7.9 Hz, 1H), 7.49 (d, J=1.7 Hz, 1H), 7.42-7.35 (m, 1H), 7.21 (d, J=1.8 Hz, 1H), 5.39 (s, 2H), 5.22 (t, J=5.6 Hz, 1H), 4.59 (dd. J=5.6, 1.1 Hz, 2H); MS (ES+): 334.0 (M+1).

### Step-5: Preparation of ethyl 2-(2-((5-bromo-7-(pyridin-2-ylmethoxy)benzofuran-3-yl)methoxy)phenyl)acetate (227f)

Compound 227f was prepared according to the procedure reported in step-2 of scheme-2 from (5-bromo-7-(pyridin-2-ylmethoxy)benzofuran-3-yl)methanol (227e) (130 mg, 0.389 mmol) in DCM (3 mL) using PPh₃ (117 mg, 0.447 mmol), ethyl 2-(2-hydroxyphenyl)acetate (2b) (77 mg, 0.428 mmol) and a solution of DCAD (164 mg, 0.447 mmol) in DCM (3 mL) to afford after workup and purification using method-AV, ethyl 2-(2-((5-bromo-7-(pyridin-2-ylmethoxy)benzofuran-3-yl)methoxy)phenyl)acetate (227f) (148 mg, 77% yield) as a white solid; MS (ES+): 496.1 (M+1).

### Step-6: Preparation of ethyl 2-(2-((7-(pyridin-2-ylmethoxy)-5-(4,4,5,5-tetramethyl-1,3,2-dioxaborolan-2-yl)benzofuran-3-yl)methoxy)phenyl)acetate (227g)

Compound 227g was prepared according to the procedure reported in step-4 of scheme-1 from ethyl 2-(2-((5-bromo-7-(pyridin-2-ylmethoxy)benzofuran-3-yl)methoxy)phenyl)acetate (227f) (0.3 g, 0.604 mmol) in anydrous dioxane (20 mL) using BISPIN (0.307 g, 1.209 mmol), KOAc (0.178 g, 1.813 mmol) and PdCl₂(dppf)-CH₂Cl₂ adduct (0.074 g, 0.091 mmol) to afford after workup and purification using method-BB, ethyl 2-(2-((7-(pyridin-2-ylmethoxy)-5-(4,4,5,5-tetramethyl-1,3,2-dioxaborolan-2-yl)benzofuran-3-yl)methoxy)phenyl)acetate (227g) (245 mg, 74.6% yield) as a clear oil; MS (ES+): 543.7 (M+1).

### Step-7: Preparation of ethyl 2-(2-((5-(3-carbamimidoylphenyl)-7-(pyridin-2-ylmethoxy)benzofuran-3-yl)methoxy)phenyl)acetate (227h)

Compound 227h was prepared according to the procedure reported in step-3 of scheme-112 from ethyl 2-(2-((7-(pyridin-2-ylmethoxy)-5-(4,4,5,5-tetramethyl-1,3,2-dioxaborolan-2-yl)benzofuran-3-yl)methoxy)phenyl)acetate (227g) (240 mg, 0.442 mmol) in dioxane (15 mL) using 3-bromobenzimidamide hydrochloride (1g) (156 mg, 0.662 mmol), K₃PO₄ (4M aqueous solution, 0.442 mL, 1.767 mmol), PCy₃ (37.2 mg, 0.132 mmol) and Pd₂(dba)₃ (40.4 mg, 0.044 mmol) to afford after workup and purification using method-F, ethyl 2-(2-((5-(3-carbamimidoylphenyl)-7-(pyridin-2-ylmethoxy)benzofuran-3-yl)methoxy)phenyl)acetate (227h) (95 mg, 0.177 mmol, 40.2% yield) as a yellow oil; MS (ES+): 535.7 (M+1).

### Step-8: Preparation of 2-(2-((5-(3-carbamimidoylphenyl)-7-(pyridin-2-ylmethoxy)benzofuran-3-yl)methoxy)phenyl)acetic acid (227i)

Compound 227i was prepared according to the procedure reported step-2 of scheme-1 from ethyl 2-(2-((5-(3-carbamimidoylphenyl)-7-(pyridin-2-ylmethoxy)benzofuran-3-yl)methoxy)phenyl)acetate (227h) (90 mg, 0.168 mmol) in THF (1 mL), ACN (0.5 mL) using a 1N solution of LiOH·H₂O (0.504 mL, 0.504 mmol) to afford after workup and purification using method-M, 2-(2-((5-(3-carbamimidoylphenyl)-7-(pyridin-2-ylmethoxy)benzofuran-3-yl)methoxy)phenyl)acetic acid (227i) (16 mg, 18.76% yield) HCl salt as a white solid; ¹H NMR (300 MHz, DMSO-d₆) δ 9.44 (s, 2H, D₂O exchangeable), 9.09 (s, 2H, D₂O exchangeable), 8.65 (d, J=4.9 Hz, 1H), 8.14 (s, 2H), 8.09 (d, J=7.7 Hz, 1H), 7.96 (t, J=7.8 Hz, 1H), 7.82-7.66 (m, 4H), 7.53 (s, 1H), 7.45 (t, J=6.4 Hz, 1H), 7.32-7.16 (m, 3H), 6.93 (t, J=7.5 Hz, 1H), 5.55 (s, 2H), 5.32 (s, 2H), 3.54 (s, 2H). MS (ES+): 508.2 (M+1); (ES−): 506.1 (M−1).

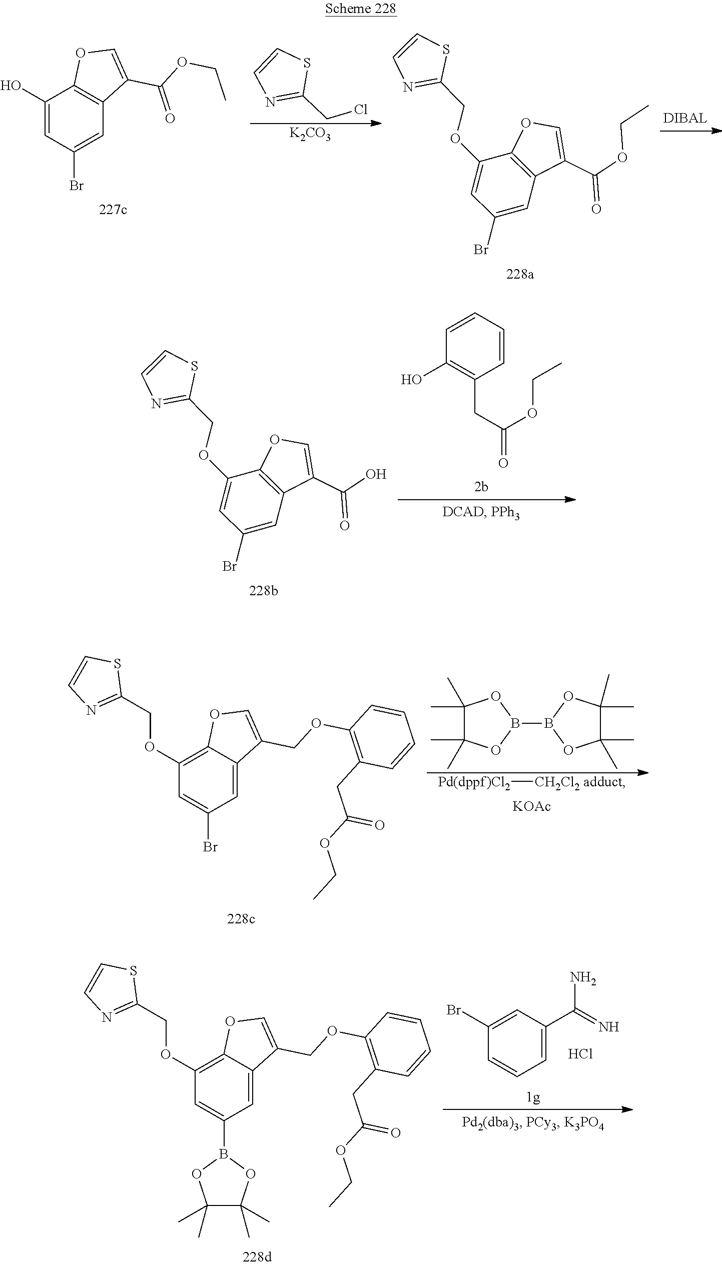

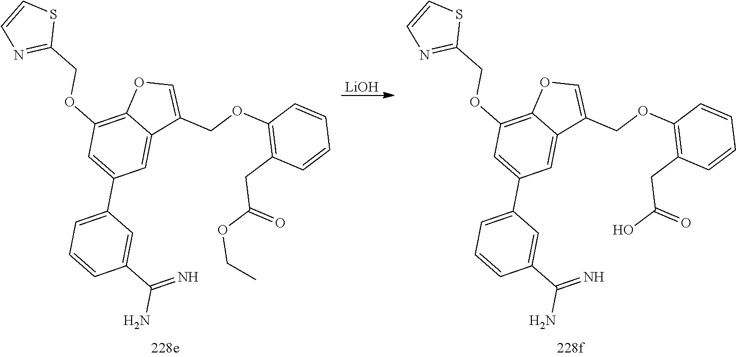

### Preparation of 2-(2-((5-(3-carbamimidoylphenyl)-7-(thiazol-2-ylmethoxy)benzofuran-3-yl)methoxy)phenyl)acetic acid (228f)

### Step-1: Preparation of ethyl 5-bromo-7-(thiazol-2-ylmethoxy)benzofuran-3-carboxylate (228a)

Compound 228a was prepared according to the procedure reported in step-3 of scheme-106 from ethyl 5-bromo-7-hydroxybenzofuran-3-carboxylate (227c) (450 mg, 1.578 mmol) in DMF (5 mL) using 2-(chloromethyl)thiazole (211 mg, 1.578 mmol; CAS #3364-78-1), K₂CO₃ (654 mg, 4.74 mmol) and stirring at RT for 14 h. This gave after workup ethyl 5-bromo-7-(thiazol-2-ylmethoxy)benzofuran-3-carboxylate (228a) (587 mg, 97% yield) as an off-white solid; ¹H NMR (300 MHz, DMSO-d₆) δ 8.83 (s, 1H), 7.90 (d, J=3.2 Hz, 1H), 7.84 (d, J=3.3 Hz, 1H), 7.70 (d, J=1.7 Hz, 1H), 7.47 (d, J=1.7 Hz, 1H), 5.71 (s, 2H), 4.36 (q, J=7.1 Hz, 2H), 1.35 (t, J=7.1 Hz, 3H); MS (ES+): 381.98 (M+1).

### Step-2: Preparation of (5-bromo-7-(thiazol-2-ylmethoxy)benzofuran-3-yl)methanol (228b)

Compound 228b was prepared according to the procedure and method of purification reported in step-1 of scheme-2 from ethyl 5-bromo-7-(thiazol-2-ylmethoxy)benzofuran-3-carboxylate (228a) (500 mg, 1.308 mmol) in DCM (2.61 mL) using 1.0 M solution of DIBAL in DCM (3.92 mL, 3.92 mmol) to afford after workup and purification using method-BC, (5-bromo-7-(thiazol-2-ylmethoxy)benzofuran-3-yl)methanol (228b) (0.243 g, 55% yield) as an off-white solid; 1H NMR (300 MHz, DMSO-d₆) δ 7.93 (s, 1H), 7.89 (d, J=3.2 Hz, 1H), 7.83 (d, J=3.2 Hz, 1H), 7.53 (d, J=1.7 Hz, 1H), 7.31 (d, J=1.7 Hz, 1H), 5.67 (s, 2H), 5.23 (t, J=5.6 Hz, 1H), 4.59 (dd, J=5.6, 1.1 Hz, 2H); MS (ES+): 339.9 (M+1).

### Step-3: Preparation of ethyl 2-(2-((5-bromo-7-(thiazol-2-ylmethoxy)benzofuran-3-yl)methoxy)phenyl)acetate (228c)

Compound 228c was prepared according to the procedure and method of purification reported in step-2 of scheme-2 from (5-bromo-7-(thiazol-2-ylmethoxy)benzofuran-3-yl)methanol (228b) (235 mg, 0.691 mmol) in DCM (5 mL) using PPh₃ (208 mg, 0.794 mmol), ethyl 2-(2-hydroxyphenyl)acetate (2b) (137 mg, 0.760 mmol) and a solution of DCAD (292 mg, 0.794 mmol) in DCM (5 mL) to afford after workup and purification using method-AV, ethyl 2-(2-((5-bromo-7-(thiazol-2-ylmethoxy)benzofuran-3-yl)methoxy)phenyl)acetate (228c) (285 mg, 82% yield) as a white solid; ¹H NMR (300 MHz, DMSO-d₆) δ 8.14 (s, 1H), 7.89 (d, J=3.3 Hz, 1H), 7.83 (d, J=3.2 Hz, 1H), 7.48 (d, J=1.7 Hz, 1H), 7.35 (d, J=1.7 Hz, 1H), 7.33-7.26 (m, 1H), 7.23-7.15 (m, 2H), 6.97-6.88 (m, 1H), 5.69 (s, 2H), 5.22 (s, 2H), 3.94 (q, J=7.1 Hz, 2H), 3.56 (s, 2H), 0.99 (t, J=7.1 Hz, 3H); MS (ES+): 502.0 (M+1).

### Step-4: Preparation of ethyl 2-(2-((5-(4,4,5,5-tetramethyl-1,3,2-dioxaborolan-2-yl)-7-(thiazol-2-ylmethoxy)benzofuran-3-yl)methoxy)phenyl)acetate (228d)

Compound 228d was prepared according to the procedure and method of purification reported in step-4 of scheme-1 from ethyl 2-(2-((5-bromo-7-(thiazol-2-ylmethoxy)benzofuran-3-yl)methoxy)phenyl)acetate (228c) (0.3 g, 0.597 mmol) in anydrous dioxane (20 mL) using BISPIN (0.303 g, 1.194 mmol), KOAc (0.176 g, 1.791 mmol), PdCl₂(dppf)-CH₂Cl₂ adduct (0.073 g, 0.090 mmol) to afford after workup and purification using method-BB, ethyl 2-(2-((5-(4,4,5,5-tetramethyl-1,3,2-dioxaborolan-2-yl)-7-(thiazol-2-ylmethoxy)benzofuran-3-yl)methoxy)phenyl)acetate (228d) (203 mg, 61.9% yield) as a clear oil; MS (ES+): 549.7 (M+1).

### Step-5: Preparation of ethyl 2-(2-((5-(3-carbamimidoylphenyl)-7-(thiazol-2-ylmethoxy)benzofuran-3-yl)methoxy)phenyl)acetate (228e)

Compound 228e was prepared according to the procedure reported in step-3 of scheme-112 from ethyl 2-(2-((5-(4,4,5,5-tetramethyl-1,3,2-dioxaborolan-2-yl)-7-(thiazol-2-ylmethoxy)benzofuran-3-yl)methoxy)phenyl)acetate (228d) (190 mg, 0.346 mmol) in dioxane (12 mL) using 3-bromobenzimidamide hydrochloride (1g) (122 mg, 0.519 mmol), 4 M solution of K₃PO₄ (0.346 mL, 1.383 mmol), PCy₃ (29.1 mg, 0.104 mmol) and Pd₂(dba)₃ (31.7 mg, 0.035 mmol) to afford after workup and purification using method-F, ethyl 2-(2-((5-(3-carbamimidoylphenyl)-7-(thiazol-2-ylmethoxy)benzofuran-3-yl)methoxy)phenyl)acetate (228e) (43 mg, 23% yield) as a yellow oil; ¹H NMR (300 MHz, DMSO-d₆) δ 8.14 (s, 1H), 8.07 (s, 1H), 7.90-7.79 (m, 3H), 7.75 (d, J=7.9 Hz, 1H), 7.62 (s, 1H), 7.55 (t, J=7.7 Hz, 1H), 7.50 (s, 1H), 7.28 (d, J=8.3 Hz, 1H), 7.25-7.15 (m, 2H), 6.93 (t, J=7.4 Hz, 1H), 5.78 (s, 2H), 5.29 (s, 2H), 3.73 (q, J=7.1 Hz, 2H), 3.56 (s, 2H), 0.85 (t, J=7.1 Hz, 3H); MS (ES+): 541.7 (M+1).

### Step-6: Preparation of 2-(2-((5-(3-carbamimidoylphenyl)-7-(thiazol-2-ylmethoxy)benzofuran-3-yl)methoxy)phenyl)acetic acid (228f)

Compound 228f was prepared according to the procedure reported step-2 of scheme-1 from ethyl 2-(2-((5-(3-carbamimidoylphenyl)-7-(thiazol-2-ylmethoxy)benzofuran-3-yl)methoxy)phenyl)acetate (228e) (40 mg, 0.074 mmol) in THF (0.5 mL), acetonitrile (0.25 mL) using a 1N solution of LiOH·H₂O (0.222 mL, 0.222 mmol) to afford after workup and purification using method-M, 2-(2-((5-(3-carbamimidoylphenyl)-7-(thiazol-2-ylmethoxy)benzofuran-3-yl)methoxy)phenyl)acetic acid (228f) (18 mg, 48% yield) HCl salt as a white solid; ¹H NMR (300 MHz, DMSO-d₆) δ 9.44 (s, 2H, D₂O exchangeable), 9.10 (s, 2H, D₂O exchangeable), 8.20-8.14 (m, 2H), 8.11 (d, J=7.8 Hz, 1H), 7.89 (d, J=3.2 Hz, 1H), 7.83 (d, J=3.2 Hz, 1H), 7.81-7.67 (m, 3H), 7.59 (d, J=1.6 Hz, 1H), 7.32-7.18 (m, 3H), 6.98-6.87 (m, 1H), 5.79 (s, 2H), 5.32 (s, 2H), 3.54 (s, 2H). MS (ES+): 514.1 (M+1); (ES−): 512.1 (M−1).

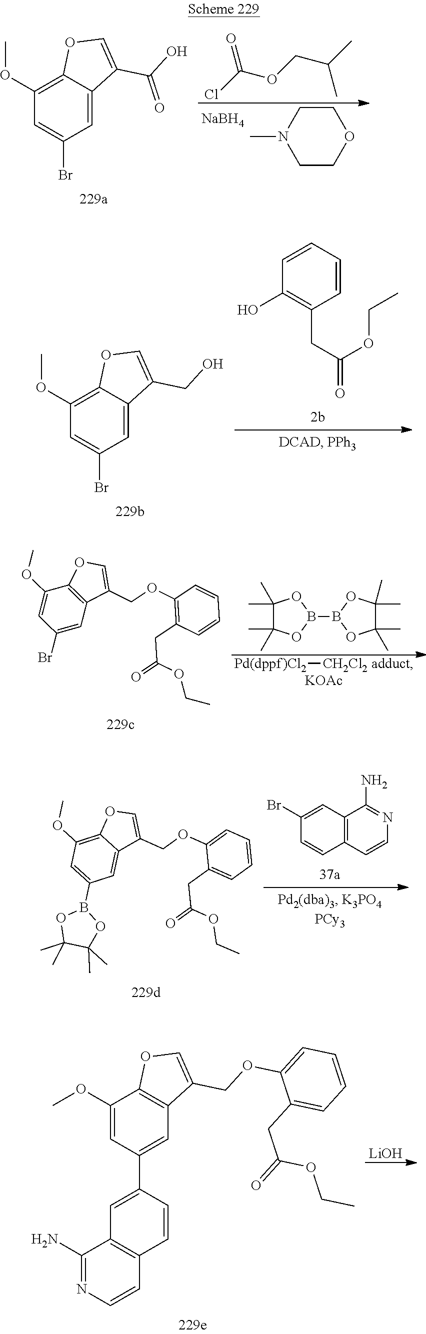

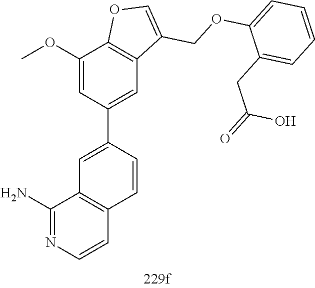 [SMILES: COc1cc(-c2ccc3ccnc(N)c3c2)cc2c(COc3ccccc3CC(=O)O)coc12]

### Preparation of 2-(2-((5-(1-aminoisoquinolin-7-yl)-7-methoxybenzofuran-3-yl)methoxy)phenyl)acetic acid (229f)

### Step-1: Preparation of (5-bromo-7-methoxybenzofuran-3-yl)methanol (229b)

Compound 229b was prepared according to the procedure and method of purification reported in step-1 of scheme-12 from 5-bromo-7-methoxybenzofuran-3-carboxylic acid (229a) (880 mg, 3.25 mmol; CAS #: 1875597-44-6) in THF (20 mL) using N-Methylmorpholine (0.428 mL, 3.90 mmol), isobutyl chloroformate (0.512 mL, 3.90 mmol) a solution of NaBH₄ (368 mg, 9.74 mmol) in water (2.0 mL). This gave after workup and purification using method-AW, (S-bromo-7-methoxybenzofuran-3-yl)methanol (229b) (458 mg, 55% yield) as a white solid; ¹H NMR (300 MHz, DMSO-d₆) δ 7.89 (s, 1H), 7.46 (d, J=1.8 Hz, 1H), 7.09 (d, J=1.8 Hz, 1H), 5.20 (t, J=5.6 Hz, 1H), 4.57 (dd, J=5.6, 1.1 Hz, 2H), 3.94 (s, 3H).

### Step-2: Preparation of ethyl 2-(2-((5-bromo-7-methoxybenzofuran-3-yl)methoxy)phenyl)acetate (229c)

Compound 229c was prepared according to the procedure and method of purification reported in step-2 of scheme-2 from (5-bromo-7-methoxybenzofuran-3-yl)methanol (229b) (300 mg, 1.167 mmol) in DCM (10 mL) using PPh₃ (352 mg, 1.342 mmol), ethyl 2-(2-hydroxyphenyl)acetate (2b) (231 mg, 1.284 mmol) and a solution of DCAD (493 mg, 1.342 mmol) in DCM (5 mL) to afford after workup and purification using method-BD, ethyl 2-(2-((5-bromo-7-methoxybenzofuran-3-yl)methoxy)phenyl)acetate (229c) (365 mg, 74.6% yield) as a white solid. ¹H NMR (300 MHz, DMSO-d₆) δ 8.10 (s, 1H), 7.42 (d, J=1.8 Hz, 1H), 7.36-7.12 (m, 4H), 6.97-6.89 (m, 1H), 5.21 (s, 2H), 4.01-3.90 (m, 5H), 3.56 (s, 2H), 1.01 (t, J=7.1 Hz, 3H); MS (ES+): 441.1/443.0 (M+Na).

### Step-3: Preparation of ethyl 2-(2-((7-methoxy-5-(4,4,5,5-tetramethyl-1,3,2-dioxaborolan-2-yl)benzofuran-3-yl)methoxy)phenyl)acetate (229d)

Compound 229d was prepared according to the procedure and method of purification reported in step-4 of scheme-1 from ethyl 2-(2-((5-bromo-7-methoxy benzofuran-3-yl)methoxy)phenyl)acetate (229c) (350 mg, 0.835 mmol) in anhydrous dioxane (30 mL) using BISPIN (424 mg, 1.670 mmol), KOAc (246 mg, 2.504 mmol) and PdCl₂(dppf)-CH₂Cl₂ adduct (102 mg, 0.125 mmol) to afford after workup and purification using method-L, ethyl 2-(2-((7-methoxy-5-(4,4,5,5-tetramethyl-1,3,2-dioxaborolan-2-yl)benzofuran-3-yl)methoxy)phenyl)acetate (229d) (217 mg, 55.7% yield) as a clear oil; MS (ES+): 489.2 (M+Na).

### Step-4: Preparation of ethyl 2-(2-((5-(1-aminoisoquinolin-7-yl)-7-methoxybenzofuran-3-yl)methoxy)phenyl)acetate (229e)

Compound 229e was prepared according to the procedure reported in step-3 of scheme-112 from ethyl 2-(2-((7-methoxy-5-(4,4,5,5-tetramethyl-1,3,2-dioxaborolan-2-yl)benzofuran-3-yl)methoxy)phenyl)acetate (229d) (200 mg, 0.429 mmol) in dioxane (15 mL) using 7-bromoisoquinolin-1-amine (37a) (191 mg, 0.858 mmol). K₃PO₄ (4M aqueous solution, 0.429 mL, 1.716 mmol), PCy₃ (36.1 mg, 0.129 mmol), and Pd₂(dba)₃ (58.9 mg, 0.064 mmol) to afford after workup and purification using method-E, ethyl 2-(2-((5-(1-aminoisoquinolin-7-yl)-7-methoxybenzofuran-3-yl)methoxy)phenyl)acetate (229e) (195 mg, 94% yield) as a clear oil; MS (ES+): 483.2 (M+1).

### Step-5: Preparation of 2-(2-((5-(1-aminoisoquinolin-7-yl)-7-methoxybenzofuran-3-yl)methoxy)phenyl)acetic acid (229f)

Compound 229f was prepared according to the procedure reported step-2 of scheme-1 from ethyl 2-(2-((5-(1-aminoisoquinolin-7-yl)-7-methoxybenzofuran-3-yl)methoxy)phenyl)acetate (229e) (190 mg, 0.394 mmol) in THF (2.4 mL), ACN (1.2 mL) using a 1N solution of LiOH·H₂O (1.181 mL, 1.181 mmol) to afford after workup and purification using method-G, 2-(2-((5-(1-aminoisoquinolin-7-yl)-7-methoxybenzofuran-3-yl)methoxy)phenyl)acetic acid (229f) (50 mg, 27.9% yield) hydrochloride salt as a white solid; ¹H NMR (300 MHz, DMSO-d₆) δ 13.19 (s, 1H, D₂O exchangeable), 12.19 (s, 1H, D₂O exchangeable), 9.18 (s, 2H, DO exchangeable), 8.96 (s, 1H), 8.42 (dd, J=8.5, 1.7 Hz, 1H), 8.13 (s, 1H), 8.06 (d, J=8.5 Hz, 1H), 7.79 (d, J=1.5 Hz, 1H), 7.70 (d, J=6.9 Hz, 1H), 7.48 (d, J=1.6 Hz, 1H), 7.33-7.16 (m, 4H), 7.05-6.73 (m, 1H), 5.34 (s, 2H), 4.12 (s, 3H), 3.55 (s, 2H). MS (ES+): 455.1 (M+1); (ES−): 453.1 (M−1).

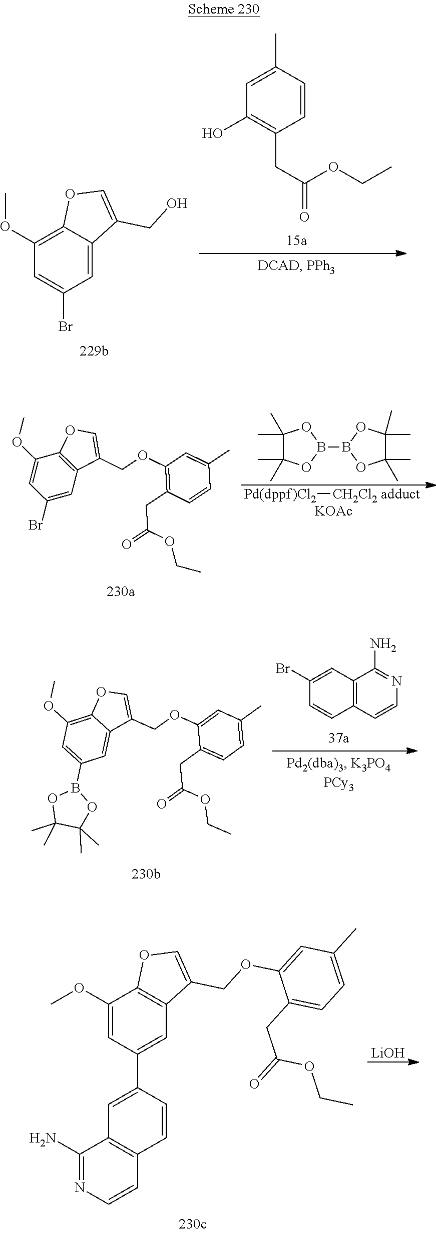

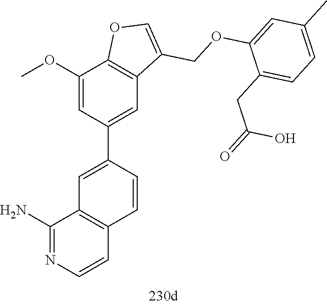

### Preparation of 2-(2-((5-(1-aminoisoquinolin-7-yl)-7-methoxybenzofuran-3-yl)methoxy)-4-methylphenyl)acetic acid (230d)

### Step-1: Preparation of ethyl 2-(2-((5-bromo-7-methoxybenzofuran-3-yl)methoxy)-4-methylphenyl)acetate (230a)

Compound 230a was prepared according to the procedure reported in step-2 of scheme-2 from (5-bromo-7-methoxybenzofuran-3-yl)methanol (229b) (300 mg, 1.167 mmol) in DCM (10 mL) using PPh₃ (352 mg, 1.342 mmol), ethyl 2-(2-hydroxy-4-methylphenyl)acetate (15a) (249 mg, 1.284 mmol) and a solution of DCAD (493 mg, 1.342 mmol) in DCM (5 mL) to afford after workup and purification using method-BD, ethyl 2-(2-((5-bromo-7-methoxybenzofuran-3-yl)methoxy)-4-methylphenyl)acetate (230a) (430 mg, 85% yield) as a white solid; MS (ES+): 433.0/435.0 (M+1); 455.0/457.0 (M+Na).

### Step-2: Preparation of ethyl 2-(2-((7-methoxy-5-(4,4,5,5-tetramethyl-1,3,2-dioxaborolan-2-yl)benzofuran-3-yl)methoxy)-4-methylphenyl)acetate (230b)

Compound 230b was prepared according to the procedure reported in step-4 of scheme-1 from ethyl 2-(2-((5-bromo-7-methoxybenzofuran-3-yl)methoxy)-4-methylphenyl)acetate (230a) (380 mg, 0.877 mmol) in anhydrous dioxane (30 mL) using BISPIN (445 mg, 1.754 mmol), KOAc (258 mg, 2.63 mmol) and PdCl₂(dppf)-CH₂Cl₂ adduct (107 mg, 0.132 mmol) to afford after workup and purification using method-L, ethyl 2-(2-((7-methoxy-5-(4,4,5,5-tetramethyl-1,3,2-dioxaborolan-2-yl)benzofuran-3-yl)methoxy)-4-methylphenyl)acetate (230b) (320 mg, 76% yield) as a clear oil; MS (ES+): 503.2 (M+Na).

### Step-3: Preparation of ethyl 2-(2-((5-(1-aminoisoquinolin-7-yl)-7-methoxybenzofuran-3-yl)methoxy)-4-methylphenyl)acetate (230c)

Compound 230c was prepared according to the procedure reported in step-3 of scheme-112 from ethyl 2-(2-((7-methoxy-5-(4,4,5,5-tetramethyl-1,3,2-dioxaborolan-2-yl)benzofuran-3-yl)methoxy)-4-methylphenyl)acetate (230b) (0.3 g, 0.625 mmol) in dioxane (20 mL) using 7-bromoisoquinolin-1-amine (37a) (0.223 g, 0.999 mmol), K₃PO₄ (4M aqueous solution, 0.625 mL, 2.498 mmol). PCy₃ (0.053 g, 0.187 mmol), and Pd₂(dba)₃ (0.086 g, 0.094 mmol) to afford after workup and purification using method-AM, ethyl 2-(2-((5-(1-aminoisoquinolin-7-yl)-7-methoxybenzofuran-3-yl)methoxy)-4-methylphenyl)acetate (230c) (245 mg, 79% yield) as a clear oil. MS (ES+): 497.2 (M+1).

### Step 4: Preparation of 2-(2-((5-(1-aminoisoquinolin-7-yl)-7-methoxybenzofuran-3-yl)methoxy)-4-methylphenyl)acetic acid (230d)

Compound 230d was prepared according to the procedure reported step-2 of scheme-1 from ethyl 2-(2-((5-(1-aminoisoquinolin-7-yl)-7-methoxybenzofuran-3-yl)methoxy)-4-methylphenyl)acetate (230c) (240 mg, 0.483 mmol) in THF (3 mL). ACN (1.5 mL) using a 1N solution of LiOH·H₂O (1.45 mL, 1.45 mmol) to afford after workup and purification using method-G, 2-(2-((5-(1-aminoisoquinolin-7-yl)-7-methoxybenzofuran-3-yl)methoxy)-4-methylphenyl)acetic acid (230d) (116 mg, 51.2% yield) hydrochloride salt as a pale yellow solid; ¹H NMR (300 MHz, DMSO-d₆) δ 13.31 (s, 1H, DO exchangeable), 12.14 (s, 1H, D₂O exchangeable), 9.29 (s, 2H, D₂O exchangeable), 9.00 (s, 1H), 8.43 (dd, J=8.5, 1.6 Hz, 1H), 8.12 (s, 1H), 8.06 (d, J=8.5 Hz, 1H), 7.80 (d, J=1.5 Hz, 1H), 7.70 (d, J=6.9 Hz, 1H), 7.50 (d, J=1.5 Hz, 1H), 7.28 (d, J=7.0 Hz, 1H), 7.12-7.04 (m, 2H), 6.75 (d, J==7.4 Hz, 1H), 5.31 (s, 2H), 4.12 (s, 3H), 3.49 (s, 2H), 2.32 (s, 3H). MS (ES+): 469.1 (M+1); (ES−): 467.1 (M−1).

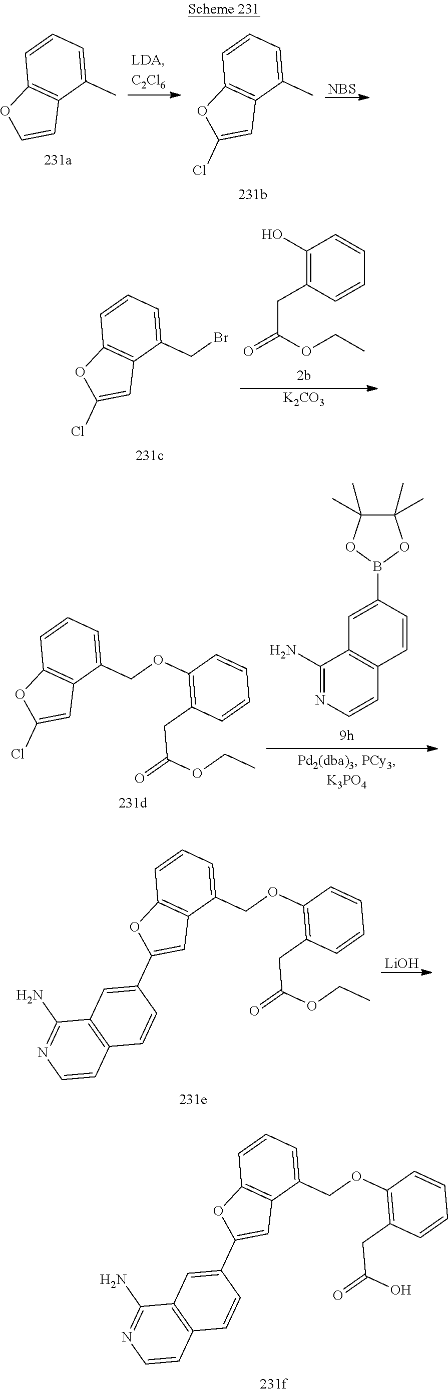

### Preparation of 2-(2-((2-(1-aminoisoquinolin-7-yl)benzofuran-4-yl)methoxy)phenyl)acetic acid (231f)

### Step-1: Preparation of 2-chloro-4-methylbenzofuran (231b)

To a solution of 4-methylbenzofuran (231a) (620 mg, 4.69 mmol; CAS #5670-23-5) in THF (12 mL) cooled to −78° C. under N₂ was added dropwise lithium diisopropylamide (LDA) (1.5 M in hexanes, 4.69 mL, 7.04 mmol) and stirred at −78° C. for 1.5 h. A solution of perchloroethane (1666 mg, 7.04 mmol) in dry THF (5 mL) was added at −78° C. and the mixture was allowed to warm to RT overnight. The solvent was evaporated, and the residue was quenched with 2 M HCl, and extracted with ether. The organic layers were combined, washed with brine, dried filtered and concentrated in vacuum. The residue obtained was purified using method-BE to afford 2-chloro-4-methylbenzofuran (231b) as a colorless oil; JH NMR (300 MHz, DMSO-d₆) δ 7.40 (d, J=8.3 Hz, 1H), 7.29-7.25 (m, 1H), 7.23-7.20 (m, 1H), 7.12 (d, J=0.9 Hz, 1H), 2.45 (s, 3H).

### Step-2: Preparation of 4-(bromomethyl)-2-chlorobenzofuran (231c)

To a solution of 2-chloro-4-methylbenzofuran (231b) (780 mg, 4.68 mmol) in CCl₄ (10 mL) was added NBS (917 mg, 5.15 mmol) and benzoyl peroxide (170 mg, 0.702 mmol) and heated at reflux overnight. The solid was removed by filtration and washed with CCl₄. The solvent was removed in vacuo to provide 4-(bromomethyl)-2-chlorobenzofuran (231c) (1.14 g, 99% yield) as a yellow oil which was used as such in the next step without further purification; MS (ES+): 245.0 (M+1).

### Step-3: Preparation of ethyl 2-(2-((2-chlorobenzofuran-4-yl)methoxy)phenyl)acetate (231d)

Compound 231d was prepared according to the procedure reported in step-3 of scheme-106 from 4-(bromomethyl)-2-chlorobenzofuran (231c) (1.1 g, 4.48 mmol) in acetone (12 mL) using K₂CO₃ (1.858 g, 13.44 mmol), ethyl 2-(2-hydroxyphenyl)acetate (2b) (0.888 g, 4.93 mmol) and heating at 45° C. for 2 h. This gave after workup and purification using method-BF, ethyl 2-(2-((2-chlorobenzofuran-4-yl)methoxy)phenyl)acetate (231d) (188 mg, 12.17% yield) as an orange oil; ¹H NMR (300 MHz, DMSO-d₆) δ 7.62-7.55 (m, 1H), 7.39-7.34 (m, 2H), 7.28-7.20 (m, 2H), 7.16-7.09 (m, 2H), 6.93 (td, J=7.4, 1.2 Hz, 1H), 5.32 (s, 2H), 3.97 (q, J=7.1 Hz, 2H), 3.61 (s, 2H), 1.06 (t, J=7.1 Hz, 3H).

### Step-4: Preparation of ethyl 2-(2-((2-(1-aminoisoquinolin-7-yl)benzofuran-4-yl)methoxy)phenyl)acetate (231e)

Compound 231e was prepared according to the procedure reported in step-3 of scheme-112 from ethyl 2-(2-((2-chlorobenzofuran-4-yl)methoxy)phenyl)acetate (231d) (180 mg, 0.522 mmol) in dioxane/2Me-THF (9 mL) using 7-(4,4,5,5-tetramethyl-1,3,2-dioxaborolan-2-yl)isoquinolin-1-amine (9h) (212 mg, 0.783 mmol), K₃PO₄ (4M aqueous solution, 0.522 mL, 2.088 mmol), Pd₂(dba)₃ (96 mg, 0.104 mmol) and PCy₃ (58.6 mg, 0.209 mmol) to afford after workup and purification using method-BG, ethyl 2-(2-((2-(1-aminoisoquinolin-7-yl)benzofuran-4-yl)methoxy)phenyl)acetate (231e) (110 mg, 46.6% yield) as a yellow solid; MS (ES+): 453.2 (M+1).

### Step-5: Preparation of 2-(2-((2-(1-aminoisoquinolin-7-yl)benzofuran-4-yl)methoxy)phenyl)acetic acid (231f)

Compound 231f was prepared according to the procedure reported step-2 of scheme-1 from ethyl 2-(2-((2-(1-aminoisoquinolin-7-yl)benzofuran-4-yl)methoxy)phenyl)acetate (231e) (108 mg, 0.239 mmol) in MeOH/THF (6 mL, 1:1) using a solution of LiOH·H₂O (50 mg, 1.192 mmol) in water (2 mL) to afford after workup and purification using method-G, 2-(2-((2-(1-aminoisoquinolin-7-yl)benzofuran-4-yl)methoxy)phenyl)acetic acid (231f) (65 mg, 64.2% yield) HCl salt as a white solid; ¹H NMR (300 MHz, DMSO-d₆) δ 12.33 (s, 2H, D₂O exchangeable), 9.25 (s, 2H, D₂O exchangeable), 9.14 (s, 1H), 8.47 (dd, J=8.5, 1.5 Hz, 1H), 8.07 (d, J=8.6 Hz, 1H), 7.85 (s, 1H), 7.74 (d, J=6.9 Hz, 1H), 7.70-7.59 (m, 1H), 7.50-7.36 (m, 2H), 7.34-7.21 (m, 3H), 7.16 (d, J=8.1 Hz, 1H), 6.94 (t, J=7.3 Hz, 1H), 5.43 (s, 2H), 3.61 (s, 2H); ¹H NMR (300 MHz, DMSO-d₆) δ 9.06 (s, 1H), 8.44 (dd, J=8.5, 1.7 Hz, 1H), 8.04 (d, J=8.6 Hz, 1H), 7.75 (s, 1H), 7.71-7.57 (m, 2H), 7.41 (q, J=4.0, 3.4 Hz, 2H), 7.23 (dd, J=8.5, 6.5 Hz, 3H), 7.14 (d, J=8.1 Hz, 1H), 6.93 (t, J=7.3 Hz, 1H), 5.40 (s, 2H), 3.59 (s, 2H); MS (ES+): 425.2 (M+1); (ES−): 423.1 (M−1).

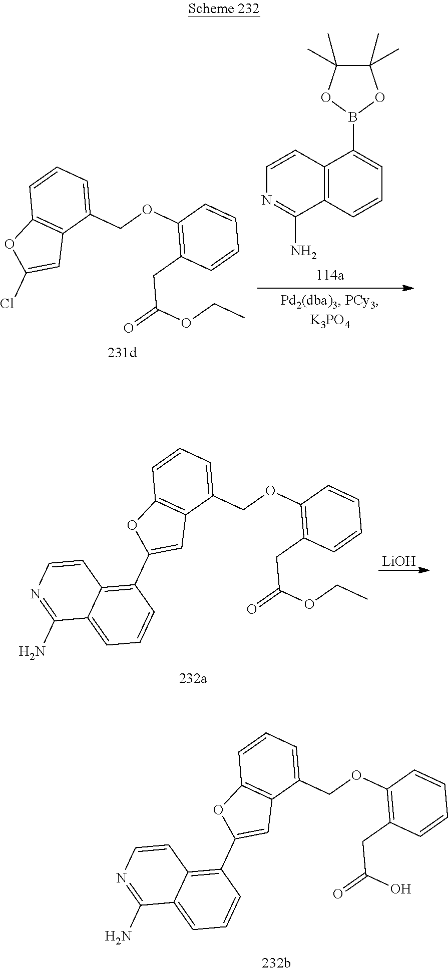

### Preparation of 2-(2-((2-(1-aminoisoquinolin-5-yl)benzofuran-4-yl)methoxy)phenyl)acetic acid (232b)

### Step-1: Preparation of ethyl 2-(2-((2-(1-aminoisoquinolin-5-yl)benzofuran-4-yl)methoxy)phenyl)acetate (232a)

Compound 232a was prepared according to the procedure reported in step-3 of scheme-112 from ethyl 2-(2-((2-chlorobenzofuran-4-yl)methoxy)phenyl)acetate (231d) (190 mg, 0.551 mmol) in Dioxane/2Me-THF (9 mL, 2:1) using 5-(4,4,5,5-tetramethyl-1,3,2-dioxaborolan-2-yl)isoquinolin-1-amine (114a) (223 mg, 0.827 mmol), K₃PO₄ (4M aqueous solution, 0.551 mL, 2.204 mmol), Pd₂(dba)₃ (101 mg, 0.110 mmol), and PCy₃ (61.8 mg, 0.220 mmol) to afford after workup and purification using method-BG, ethyl 2-(2-((2-(1-aminoisoquinolin-5-yl)benzofuran-4-yl)methoxy)phenyl)acetate (232a) (156 mg, 62.6% yield) as a yellow oil; MS (ES+): 453.2 (M+1).

### Step-2: Preparation of 2-(2-((2-(1-aminoisoquinolin-5-yl)benzofuran-4-yl)methoxy)phenyl)acetic acid (232b)

Compound 232b was prepared according to the procedure reported step-2 of scheme-1 from ethyl 2-(2-((2-(1-aminoisoquinolin-5-yl)benzofuran-4-yl)methoxy)phenyl)acetate (232a) (156 mg, 0.345 mmol) in MeOH/THF (6 mL, 1:1) using a solution of LiOH·H₂O (70 mg, 1.668 mmol) in water (2 mL) to afford after workup and purification using method-G, 2-(2-((2-(1-aminoisoquinolin-5-yl)benzofuran-4-yl)methoxy)phenyl)acetic acid (232b) (69 mg, 47.2% yield) as a yellow solid; ¹H NMR (300 MHz, DMSO-d₆) δ 13.62 (s, 1H, D₂O exchangeable), 12.20 (s, 1H, D₂O exchangeable), 9.36 (s, 2H, D₂O exchangeable), 8.73 (d, J=8.4 Hz, 1H), 8.38 (d, J=7.5 Hz, 1H), 7.91 (t, J=7.9 Hz, 1H), 7.80 (d, J=7.4 Hz, 1H), 7.67 (dd, J=7.6, 4.6 Hz, 2H), 7.62 (s, 1H), 7.49-7.38 (m, 2H), 7.29-7.13 (m, 3H), 6.92 (t, J=7.3 Hz, 1H), 5.46 (s, 2H), 3.60 (s, 2H); ¹H NMR (300 MHz, DMSO-d₆) δ 8.58 (d, J=8.3 Hz, 1H), 8.35 (d, J=7.6 Hz, 1H), 7.88 (t, J=8.0 Hz, 1H), 7.68 (p, J=7.0 Hz, 3H), 7.52 (d, J=4.5 Hz, 1H), 7.40 (d, J=7.2 Hz, 2H), 7.28-7.10 (m, 3H), 6.91 (t, J=7.4 Hz, 1H), 5.41 (s, 2H), 3.57 (s, 2H); MS (ES+): 425.2 (M+1); (ES−): 423.1 (M−1).

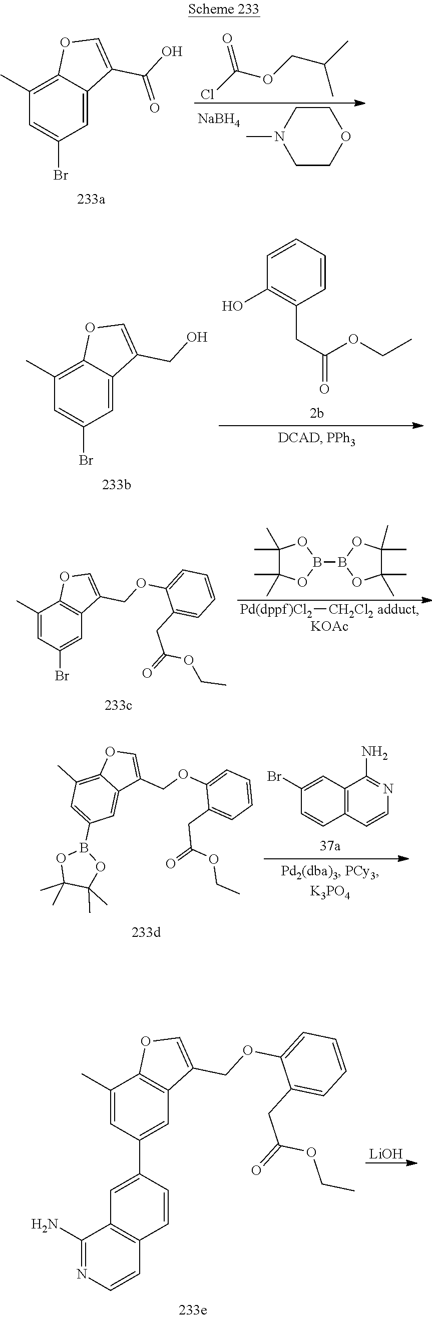

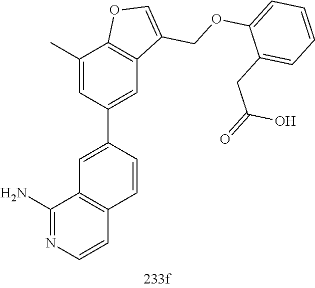 [SMILES: Cc1cc(-c2ccc3ccnc(N)c3c2)cc2c(COc3ccccc3CC(=O)O)coc12]

### Preparation of 2-(2-((5-(1-aminoisoquinolin-7-yl)-7-methylbenzofuran-3-yl)methoxy)phenyl)acetic acid (233f)

### Step-1: Preparation of (5-bromo-7-methylbenzofuran-3-yl)methanol (233b)

Compound 233b was prepared according to the procedure and method of purification reported in step-1 of scheme-12 from 5-bromo-7-methylbenzofuran-3-carboxylic acid (233a) (750 mg, 2.94 mmol; CAS #: 1492450-22-2) in THF (20 mL) using N-Methylmorpholine (0.388 mL, 3.53 mmol), isobutyl chloroformate (0.463 mL, 3.53 mmol) a solution of NaBH₄ (334 mg, 8.82 mmol) in water (2.0 mL). This gave after workup and purification using method-BH, (5-bromo-7-methylbenzofuran-3-yl)methanol (233b) (602 mg, 85% yield) as a white solid; ¹H NMR (300 MHz, DMSO-d₆) δ 7.91 (s, 1H), 7.70 (d, J=2.1 Hz, 1H), 7.32 (d, J=2.1 Hz, 1H), 5.19 (t, J=5.6 Hz, 1H), 4.59 (dd, J=5.6, 1.1 Hz, 2H), 2.45 (s, 3H).

### Step-2: Preparation of ethyl 2-(2-((5-bromo-7-methylbenzofuran-3-yl)methoxy)phenyl)acetate (233c)

Compound 233c was prepared according to the procedure and method of purification reported in step-2 of scheme-2 from (5-bromo-7-methylbenzofuran-3-yl)methanol (233b) (286 mg, 1.186 mmol) in DCM (20 mL) using PPh₃ (342 mg, 1.305 mmol), ethyl 2-(2-hydroxyphenyl)acetate (2b) (235 mg, 1.305 mmol) and a solution of DCAD (479 mg, 1.305 mmol) in DCM (20 mL) to afford after workup and purification using method-V, ethyl 2-(2-((S-bromo-7-methylbenzofuran-3-yl)methoxy)phenyl)acetate (233c) (242 mg, 51% yield) as a yellow oil; ¹H NMR (300 MHz, DMSO-d₆) δ 8.14 (s, 1H), 7.67 (d, J=2.0 Hz, 1H), 7.37 (dd. J=2.0, 1.0 Hz, 1H), 7.29 (ddd, J=9.0, 7.4, 1.8 Hz, 1H), 7.20 (td, J=8.1, 1.4 Hz, 2H), 6.93 (td, J=7.4, 1.2 Hz, 1H), 5.23 (d, J=0.9 Hz, 2H), 3.95 (q, J=7.1 Hz, 2H), 3.56 (s, 2H), 2.47 (s, 3H), 1.00 (t, J=7.1 Hz, 3H).

### Step-3: Preparation of ethyl 2-(2-((7-methyl-5-(4,4,5,5-tetramethyl-1,3,2-dioxaborolan-2-yl)benzofuran-3-yl)methoxy)phenyl)acetate (233d)

Compound 233d was prepared according to the procedure and method of purification reported in step-4 of scheme-1 from ethyl 2-(2-((5-bromo-7-methylbenzofuran-3-yl)methoxy)phenyl)acetate (233c) (350 mg, 0.868 mmol) in anhydrous dioxane (30 mL) using BISPIN (441 mg, 1.736 mmol) KOAc (256 mg, 2.60 mmol) and PdCl₂(dppf)-CH₂Cl₂ adduct (106 mg, 0.130 mmol) to afford after workup and purification using method-B, ethyl 2-(2-((7-methyl-5-(4,4,5,5-tetramethyl-1,3,2-dioxaborolan-2-yl)benzofuran-3-yl)methoxy)phenyl)acetate (233d) (330 mg, 0.733 mmol, 84% yield) as a clear oil; MS (ES+): 473.2 (M+Na).

### Step-4: Preparation of ethyl 2-(2-((5-(1-aminoisoquinolin-7-yl)-7-methylbenzofuran-3-yl)methoxy)phenyl)acetate (233e)

Compound 233e was prepared according to the procedure reported in step-3 of scheme-112 from ethyl 2-(2-((7-methyl-5-(4,4,5,5-tetramethyl-1,3,2-dioxaborolan-2-yl)benzofuran-3-yl)methoxy)phenyl)acetate (233d) (0.285 g, 0.633 mmol) in dioxane (20 mL) using 7-bromoisoquinolin-1-amine (37a) (0.226 g, 1.013 mmol), K₃PO₄ (4M aqueous solution, 0.633 mL, 2.53 mmol), PCy₃ (0.053 g, 0.190 mmol) and Pd₂(dba)₃ (0.087 g, 0.095 mmol) to afford after workup and purification using method-AM, ethyl 2-(2-((5-(1-aminoisoquinolin-7-yl)-7-methylbenzofuran-3-yl)methoxy)phenyl)acetate (233e) (225 mg, 76% yield) as a clear oil; MS (ES+): 467.2 (M+1).

### Step-5: Preparation of 2-(2-((5-(1-aminoisoquinolin-7-yl)-7-methylbenzofuran-3-yl)methoxy)phenyl)acetic acid (233f)

Compound 233f was prepared according to the procedure reported step-2 of scheme-1 from ethyl 2-(2-((5-(1-aminoisoquinolin-7-yl)-7-methylbenzofuran-3-yl)methoxy)phenyl)acetate (233e) (220 mg, 0.472 mmol) in THF (3 mL), ACN (1.5 mL) using a 1N solution of LiOH·H₂O (1.415 mL, 1.415 mmol) to afford after workup and purification using method-G, 2-(2-((5-(1-aminoisoquinolin-7-yl)-7-methylbenzofuran-3-yl)methoxy)phenyl)acetic acid (233f) (65 mg, 31.4% yield) HCl salt as an off white solid; ¹H NMR (300 MHz, DMSO-d₆) δ 9.15 (s, 2H, D₂O exchangeable), 8.94 (s, 1H), 8.39 (dd, J=8.5, 1.6 Hz, 1H), 8.16 (s, 1H), 8.09-8.02 (m, 2H), 7.76 (s, 1H), 7.70 (d, J=6.9 Hz, 1H), 7.31-7.18 (m, 4H), 6.93 (td, J=7.2, 1.3 Hz, 1H), 5.35 (s, 2H), 3.55 (s, 2H), 2.60 (s, 3H). MS (ES+): 439.2 (M+1); (ES−): 437.1 (M−1).

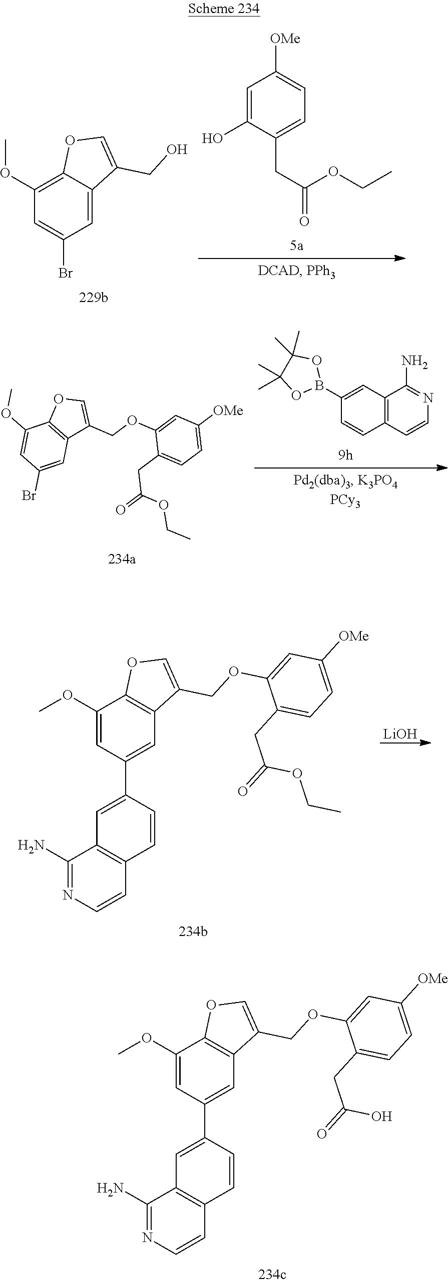

### Preparation of 2-(2-((5-(1-aminoisoquinolin-7-yl)-7-methoxybenzofuran-3-yl)methoxy)-4-methoxyphenyl)acetic acid (234c)

### Step-1: Preparation of ethyl 2-(2-((5-bromo-7-methoxybenzofuran-3-yl)methoxy)-4-methoxyphenyl)acetate (234a)

Compound 234a was prepared according to the procedure and method of purification reported in step-2 of scheme-2 from (5-bromo-7-methoxybenzofuran-3-yl)methanol (229b) (350 mg, 1.361 mmol) in DCM (10 mL) using PPh₃ (411 mg, 1.566 mmol), ethyl 2-(2-hydroxy-4-methoxyphenyl)acetate (5a) (315 mg, 1.498 mmol) and a solution of DCAD (575 mg, 1.566 mmol) in DCM (5 mL) to afford after workup and purification using method-BD, ethyl 2-(2-((5-bromo-7-methoxybenzofuran-3-yl)methoxy)-4-methoxyphenyl)acetate (234a) (310 mg, 50.7% yield) as a white solid; ¹H NMR (300 MHz, DMSO-d₆) δ 8.10 (s, 1H), 7.42 (d, J=1.7 Hz, 1H), 7.15 (d, J=1.7 Hz, 1H), 7.10 (d, J=8.3 Hz, 1H), 6.77 (d, J=2.4 Hz, 1H), 6.50 (dd. J=8.3, 2.4 Hz, 1H), 5.21 (s, 2H), 4.00-3.89 (m, 5H), 3.77 (s, 3H), 3.47 (s, 2H), 1.01 (t, J=7.1 Hz, 3H); MS (ES+): 471.0/473.0 (M+Na).

### Step-2: Preparation of ethyl 2-(2-((5-(1-aminoisoquinolin-7-yl)-7-methoxybenzofuran-3-yl)methoxy)-4-methoxyphenyl)acetate (234b)

Compound 234b was prepared according to the procedure reported in step-3 of scheme-112 from ethyl 2-(2-((5-bromo-7-methoxybenzofuran-3-yl)methoxy)-4-methoxyphenyl)acetate (234a) (150 mg, 0.334 mmol) in dioxane (12 mL) using 7-(4,4,5,5-tetramethyl-1,3,2-dioxaborolan-2-yl)isoquinolin-1-amine (9h) (135 mg, 0.501 mmol), K₃PO₄ (4M aqueous solution, 0.334 mL, 1.335 mmol), PCy₃ (28.1 mg, 0.100 mmol) and Pd₂(dba)₃ (45.9 mg, 0.050 mmol) to afford after workup and purification using method-E, ethyl 2-(2-((5-(1-aminoisoquinolin-7-yl)-7-methoxybenzofuran-3-yl)methoxy)-4-methoxyphenyl)acetate (234b) (101 mg, 59.0% yield) as a clear oil; MS (ES+): 513.2 (M+1).

### Step-3: Preparation of 2-(2-((5-(1-aminoisoquinolin-7-yl)-7-methoxybenzofuran-3-yl)methoxy)-4-methoxyphenyl)acetic acid (234c)

Compound 234c was prepared according to the procedure reported step-2 of scheme-1 from ethyl 2-(2-((5-(1-aminoisoquinolin-7-yl)-7-methoxybenzofuran-3-yl)methoxy)-4-methoxyphenyl)acetate (234b) (95 mg, 0.185 mmol) in THF (1.2 mL), ACN (0.6 mL) using a 1N solution of LiOH·H₂O (0.556 mL, 0.556 mmol) to afford after workup and purification using method-G, 2-(2-((5-(1-aminoisoquinolin-7-yl)-7-methoxybenzofuran-3-yl)methoxy)-4-methoxyphenyl)acetic acid (234c) (48 mg, 53.5% yield) HCl salt as an off white solid; ¹H NMR (300 MHz, DMSO-d₆) δ 13.24 (s, 1H, D₂O exchangeable), 9.26 (s, 2H, D₂O exchangeable), 8.98 (s, 1H), 8.43 (dd, J=8.5, 1.6 Hz, 1H), 8.12 (s, 1H), 8.05 (d, 1H), 7.80 (d, J=1.5 Hz, 1H), 7.70 (d, J=6.9 Hz, 1H), 7.49 (d, J=1.6 Hz, 1H), 7.29 (d, J=7.0 Hz, 1H), 7.11 (d, J=8.3 Hz, 1H), 6.81 (d, J=2.4 Hz, 1H), 6.51 (dd, J=8.3, 2.4 Hz, 1H), 5.33 (s, 2H), 4.12 (s, 3H), 3.77 (s, 3H), 3.46 (s, 2H). MS (ES+): 485.1 (M+1); (ES−): 483.1 (M−1).

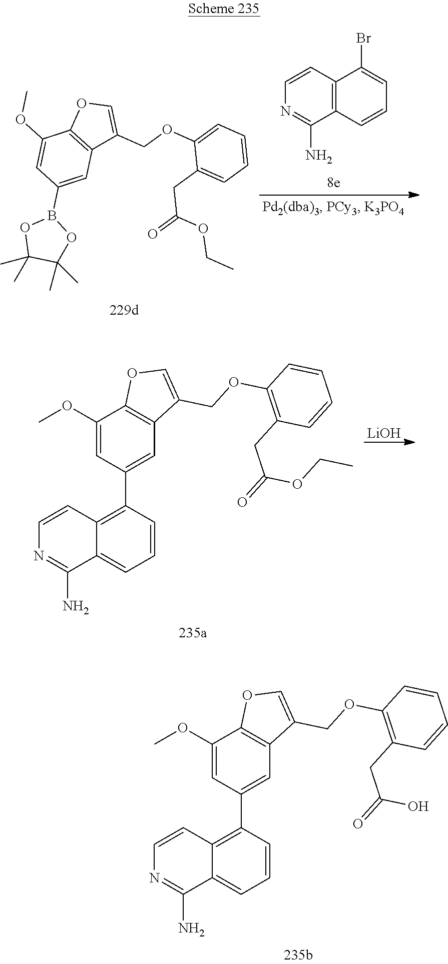

### Preparation of 2-(2-((5-(1-aminoisoquinolin-5-yl)-7-methoxybenzofuran-3-yl)methoxy)phenyl)acetic acid (235b)

### Step-1: Preparation of ethyl 2-(2-((5-(1-aminoisoquinolin-5-yl)-7-methoxybenzofuran-3-yl)methoxy)phenyl)acetate (235a)

Compound 235a was prepared according to the procedure reported in step-3 of scheme-112 from ethyl 2-(2-((7-methoxy-5-(4,4,5,5-tetramethyl-1,3,2-dioxaborolan-2-yl)benzofuran-3-yl)methoxy)phenyl)acetate (229d) (0.24 g, 0.515 mmol) in dioxane (18 mL) using 5-bromoisoquinolin-1-amine (8e) (0.172 g, 0.772 mmol), K₃PO₄ (4M aqueous solution, 0.515 mL, 2.059 mmol). PCy₃ (0.043 g, 0.154 mmol) and Pd₂(dba)₃ (0.071 g, 0.077 mmol) to afford after workup and purification using method-AM, ethyl 2-(2-((5-(1-aminoisoquinolin-5-yl)-7-methoxybenzofuran-3-yl)methoxy)phenyl)acetate (235a) (0.213 g, 86% yield) as a clear oil. ¹H NMR (300 MHz, DMSO-d₆) δ 8.23 (d, J=8.2 Hz, 1H), 8.15 (s, 1H), 7.77 (d, J=6.1 Hz, 1H), 7.68-7.57 (m, 1H), 7.54 (d, J=7.9 Hz, 1H), 7.30-7.22 (m, 2H), 7.22-7.16 (m, 2H), 7.01 (d, J=1.4 Hz, 1H), 6.91-6.82 (m, 3H), 5.26 (s, 2H), 3.97 (s, 2H), 3.95 (s, 3H), 3.67 (q, J=7.1 Hz, 2H), 3.54 (s, 2H), 0.83 (t, J=7.1 Hz, 3H). MS (ES+): 483.2 (M+1).

### Step-2: Preparation of 2-(2-((5-(1-aminoisoquinolin-5-yl)-7-methoxybenzofuran-3-yl)methoxy)phenyl)acetic acid (235b)

Compound 235b was prepared according to the procedure reported step-2 of scheme-1 from ethyl 2-(2-((5-(1-aminoisoquinolin-5-yl)-7-methoxybenzofuran-3-yl)methoxy)phenyl)acetate (235a) (190 mg, 0.394 mmol) in THF (2.4 mL), ACN (1.2 mL) using a 1N solution of LiOH·H₂O (1.181 mL, 1.181 mmol) to afford after workup and purification using method-G, 2-(2-((5-(1-aminoisoquinolin-5-yl)-7-methoxybenzofuran-3-yl)methoxy)phenyl)acetic acid (235b) (105 mg, 58.7% yield) HCl salt as a pale yellow solid; ¹H NMR (300 MHz, DMSO-d₆) δ 13.32 (s, 1H, D₂O exchangeable), 12.11 (s, 1H, D₂O exchangeable), 9.17 (s, 2H, D₂O exchangeable), 8.64 (d, J=8.3 Hz, 1H), 8.16 (s, 1H), 7.99 (dd, J=7.3, 1.1 Hz, 1H), 7.90-7.81 (m, 1H), 7.64 (d, J=7.2 Hz, 1H), 7.33 (d, J=1.4 Hz, 1H), 7.29-7.13 (m, 3H), 7.07 (d, J=7.2 Hz, 1H), 7.02 (d, J=1.4 Hz, 1H), 6.95-6.86 (m, 1H), 5.28 (s, 2H), 3.98 (s, 3H), 3.51 (s, 2H); MS (ES+): 455.1 (M+1); (ES−): 453.1 (M−1).

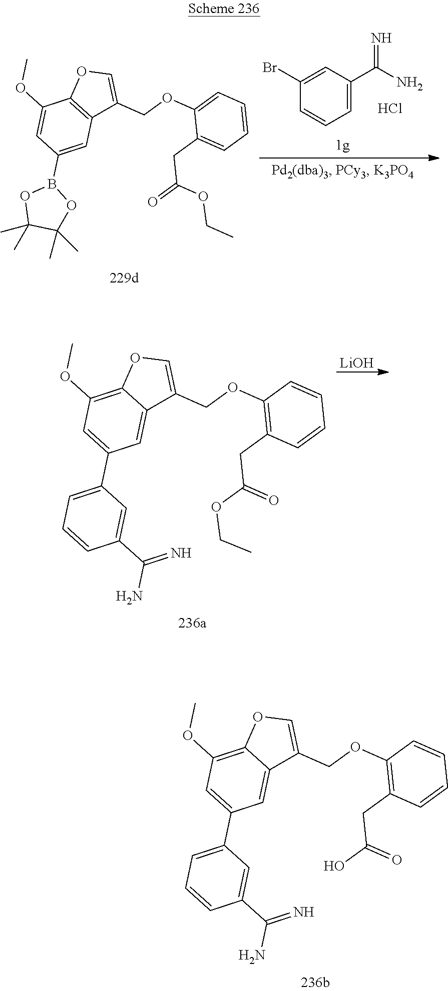

### Preparation of 2-(2-((5-(3-carbamimidoylphenyl)-7-methoxybenzofuran-3-yl)methoxy)phenyl)acetic acid (236b)

### Step-1: Preparation of ethyl 2-(2-((5-(3-carbamimidoylphenyl)-7-methoxybenzofuran-3-yl)methoxy)phenyl)acetate (236a)

Compound 236a was prepared according to the procedure reported in step-3 of scheme-112 from ethyl 2-(2-((7-methoxy-5-(4,4,5,5-tetramethyl-1,3,2-dioxaborolan-2-yl)benzofuran-3-yl)methoxy)phenyl)acetate (229d) (240 mg, 0.515 mmol) in dioxane (18 mL) using 3-bromobenzimidamide hydrochloride (1g) (182 mg, 0.772 mmol), K₃PO₄ (4M aqueous solution, 0.515 mL, 2.059 mmol), PCy₃ (43.3 mg, 0.154 mmol) and Pd₂(dba)₃ (70.7 mg, 0.077 mmol) to afford after workup and purification using method-F, ethyl 2-(2-((5-(3-carbamimidoylphenyl)-7-methoxybenzofuran-3-yl)methoxy)phenyl)acetate (236a) (71 mg, 30.1% yield) as a pale yellow solid; ¹H NMR (300 MHz, DMSO-d₆) δ 8.17-8.07 (m, 2H), 7.99 (d, J=7.6 Hz, 1H), 7.77 (d, J=7.9 Hz, 1H), 7.64 (d, J=7.8 Hz, 1H), 7.59 (d, J=1.5 Hz, 1H), 7.33 (s, 1H), 7.29 (d, J=7.5 Hz, 1H), 7.21 (dd, J=9.2, 3.5 Hz, 2H), 6.93 (t, J=7.2 Hz, 1H), 5.29 (s, 2H), 4.06 (s, 3H), 3.75 (q, J=7.0 Hz, 2H), 3.56 (s, 2H), 0.88 (t, J=7.1 Hz, 3H); MS (ES+): 459.1 (M+1).

### Step-2: Preparation of 2-(2-((5-(3-carbamimidoylphenyl)-7-methoxybenzofuran-3-yl)methoxy)phenyl)acetic acid (236b)

Compound 236b was prepared according to the procedure reported step-2 of scheme-1 from ethyl 2-(2-((5-(3-carbamimidoylphenyl)-7-methoxybenzofuran-3-yl)methoxy)phenyl)acetate (236a) (70 mg, 0.153 mmol) in THF (1 mL), ACN (0.5 mL) using a 1N solution of LiOH·H₂O (0.458 mL, 0.458 mmol) to afford after workup and purification using method-G, 2-(2-((5-(3-carbamimidoylphenyl)-7-methoxybenzofuran-3-yl)methoxy)phenyl)acetic acid (236b) (42 mg, 63.9% yield) HCl salt as a white solid; ¹H NMR (300 MHz, DMSO-d₆) δ 12.14 (s, 1H, D₂O exchangeable), 9.52 (s, 2H, D₂O exchangeable), 9.16 (s, 2H, D₂O exchangeable), 8.26-8.16 (m, 1H), 8.16-8.03 (m, 2H), 7.84-7.63 (m, 3H), 7.40 (d, J=1.5 Hz, 1H), 7.31-7.17 (m, 3H), 6.93 (td, J=7.3, 1.2 Hz, 1H), 5.31 (s, 2H), 4.07 (s, 3H), 3.54 (s, 2H); MS (ES+): 431.2 (M+1); (ES−): 429.1 (M−1).

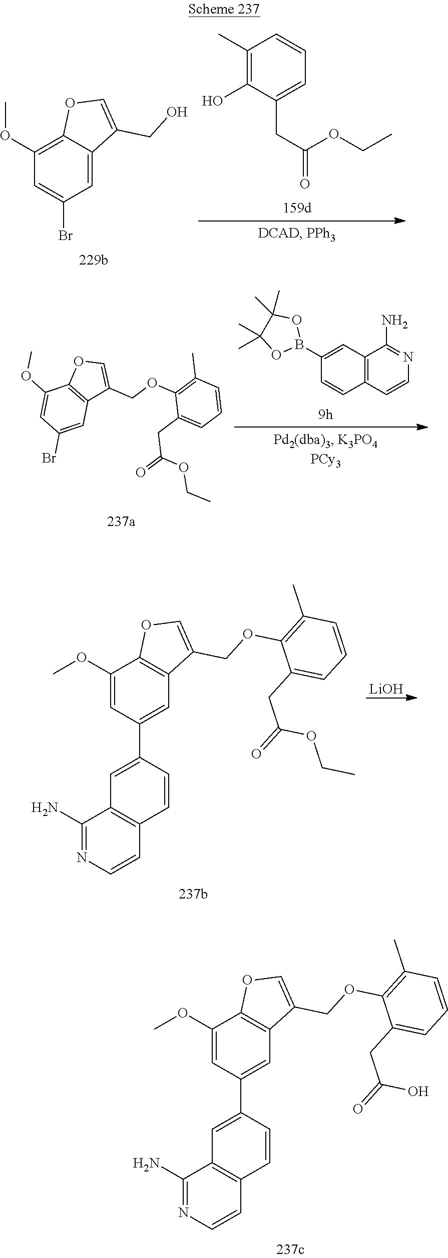

### Preparation of 2-(2-(5-(1-aminoisoquinolin-7-yl)-7-methoxybenzofuran-3-yl)methoxy)-3-methylphenyl)acetic acid (237c)

### Step-1: Preparation of ethyl 2-(2-((5-bromo-7-methoxybenzofuran-3-yl)methoxy)-3-methylphenyl)acetate (237a)

Compound 237a was prepared according to the procedure and method of purification reported in step-2 of scheme-2 from (5-bromo-7-methoxybenzofuran-3-yl)methanol (229b) (0.5 g, 1.945 mmol) in DCM (15 mL) using PPh₃ (0.587 g, 2.237 mmol), ethyl 2-(2-hydroxy-3-methylphenyl)acetate (159d) (0.416 g, 2.139 mmol) and a solution of DCAD (0.821 g, 2.237 mmol) in DCM (5 mL) to afford after workup and purification using method-BD, ethyl 2-(2-((5-bromo-7-methoxybenzofuran-3-yl)methoxy)-3-methylphenyl)acetate (237a) (430 mg, 51.0% yield) as a clear oil. ¹H NMR (300 MHz, DMSO-d₆) δ 8.11 (s, 1H), 7.43 (d, J=1.8 Hz, 1H), 7.20-6.99 (m, 4H), 4.95 (s, 2H), 4.05-3.94 (m, 5H), 3.65 (s, 2H), 2.30 (s, 3H), 1.10 (t, J=7.1 Hz, 3H). MS (ES+): 433.0 (M+1).

### Step-2: Preparation of ethyl 2-(2-((5-(1-aminoisoquinolin-7-yl)-7-methoxybenzofuran-3-yl)methoxy)-3-methylphenyl)acetate (237b)

Compound 237b was prepared according to the procedure reported in step-3 of scheme-112 from ethyl 2-(2-((5-bromo-7-methoxybenzofuran-3-yl)methoxy)-3-methylphenyl)acetate (237a) (150 mg, 0.346 mmol) in dioxane (12 mL) using 7-(4,4,5,5-tetramethyl-1,3,2-dioxaborolan-2-yl)isoquinolin-1-amine (9h) (140 mg, 0.519 mmol), potassium phosphate (0.346 mL, 1.385 mmol), PCy₃ (29.1 mg, 0.104 mmol) and Pd₂(dba)₃ (47.6 mg, 0.052 mmol) to afford after workup and purification using method-E, ethyl 2-(2-((5-(1-aminoisoquinolin-7-yl)-7-methoxybenzofuran-3-yl)methoxy)-3-methylphenyl)acetate (237b) (105 mg, 61.1% yield) as a clear oil. MS (ES+): 497.2 (M+1).

### Step-3: Preparation of 2-(2-((5-(1-aminoisoquinolin-7-yl)-7-methoxybenzofuran-3-yl)methoxy)-3-methylphenyl)acetic acid (237c)

Compound 237c was prepared according to the procedure reported step-2 of scheme-1 from ethyl 2-(2-((5-(1-aminoisoquinolin-7-yl)-7-methoxybenzofuran-3-yl)methoxy)-3-methylphenyl)acetate (237b) (100 mg, 0.201 mmol) in THF (1.2 mL), ACN (0.6 mL) using a 1N solution of LiOH·H₂O (0.604 mL, 0.604 mmol)) to afford after workup and purification using method-G, 2-(2-((5-(1-aminoisoquinolin-7-yl)-7-methoxybenzofuran-3-yl)methoxy)-3-methylphenyl)acetic acid (237c) (53 mg, 56.2% yield) HCl salt as an off white solid; 1H NMR (300 MHz, DMSO-d₆) δ 13.04 (s, 1H, D₂O exchangeable), 12.30 (s, 1H, D₂O exchangeable), 9.12 (s, 1H, D₂O exchangeable), 8.92 (s, 1H), 8.38 (d, J=8.5 Hz, 1H), 8.17 (s, 1H), 8.08 (d, J=8.5 Hz, 1H), 7.75 (s, 1H), 7.70 (d, J=6.9 Hz, 1H), 7.46 (s, 1H), 7.30 (d, J=7.0 Hz, 1H), 7.15 (t, J=8.1 Hz, 2H), 7.03 (t, J=7.5 Hz, 1H), 5.05 (s, 2H), 4.12 (s, 3H), 3.65 (s, 2H), 2.32 (s, 3H). MS (ES+): 469.1 (M+1); (ES−): 467.1 (M−1).

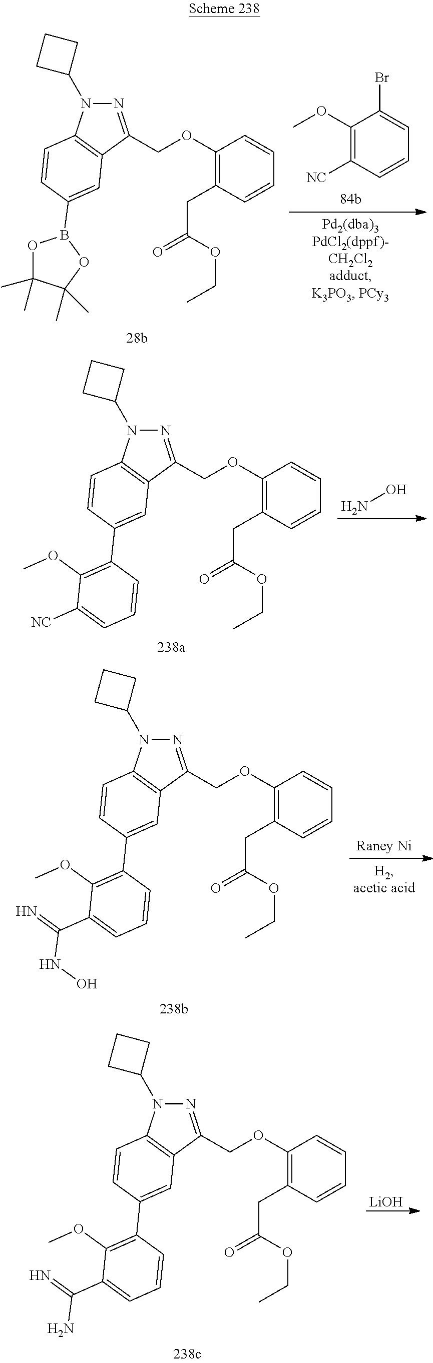

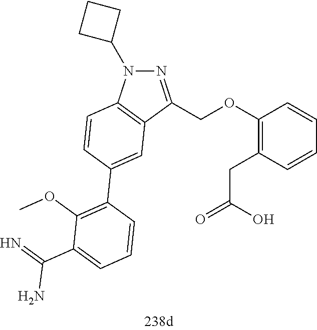 [SMILES: COc1c(C(=N)N)cccc1-c1ccc2c(c1)c(COc1ccccc1CC(=O)O)nn2C1CCC1]

### Preparation of 2-(2-((5-(3-carbamimidoyl-2-methoxyphenyl)-1-cyclobutyl-1H-indazol-3-yl)methoxy)phenyl)acetic acid (238d)

### Step-1: Preparation of ethyl 2-(2-((5-(3-cyano-2-methoxyphenyl)-1-cyclobutyl-1H-indazol-3-yl)methoxy)phenyl)acetate (238a)

Compound 238a was prepared according to the procedure reported in step-5 of scheme-1 from ethyl 2-(2-((1-cyclobutyl-5-(4,4,5,5-tetramethyl-1,3,2-dioxaborolan-2-yl)-1H-indazol-3-yl)methoxy)phenyl)acetate (28b) (350 mg, 0.714 mmol) in dioxane/THF (6 mL, 1:1) using 3-bromo-2-methoxybenzonitrile (84b) (227 mg, 1.071 mmol), K₃PO₄ (2 M aqueous solution, 1.427 mL, 2.85 mmol), PCy₃ (40.0 mg, 0.143 mmol), PdCl₂(dppf)-CH₂Cl₂ adduct (58.3 mg, 0.071 mmol) and Pd₂(dba)₃ (65.4 mg, 0.071 mmol) to afford after workup and purification using method-P, ethyl 2-(2-((5-(3-cyano-2-methoxyphenyl)-1-cyclobutyl-1H-indazol-3-yl)methoxy)phenyl)acetate (238a) (300 mg, 85% yield) as a clear oil; ¹H NMR (300 MHz, DMSO-d₆) δ 7.93-7.89 (m, 1H), 7.83-7.73 (m, 3H), 7.59 (dd, J=8.8, 1.6 Hz, 1H), 7.39 (t, J=7.7 Hz, 1H), 7.31-7.25 (m, 2H), 7.18 (d, J=7.3 Hz, 1H), 6.98-6.83 (m, 1H), 5.45 (s, 2H), 5.40-5.23 (m, 1H), 3.70-3.63 (m, 2H), 3.55 (s, 3H), 3.53 (s, 2H), 2.76-2.58 (m, 2H), 2.49 (s, 2H), 1.97-1.82 (m, 2H), 0.81 (t, J=7.1 Hz, 3H); MS (ES+): 496.20 (M+1); 518.20 (M+Na).

### Step-2: Preparation of ethyl 2-(2-((1-cyclobutyl-5-(3-(N-hydroxycarbamimidoyl)-2-methoxyphenyl)-1H-indazol-3-yl)methoxy)phenyl)acetate (238b)

Compound 238b was prepared according to the procedure reported in step-5 of scheme-23 from ethyl 2-(2-((5-(3-cyano-2-methoxyphenyl)-1-cyclobutyl-1H-indazol-3-yl)methoxy)phenyl)acetate (238a) (300 mg, 0.605 mmol) in EtOH (10 mL) using hydroxylamine (0.400 mL, 6.05 mmol) to afford after workup and purification using method-E, ethyl 2-(2-((1-cyclobutyl-5-(3-(N-hydroxycarbamimidoyl)-2-methoxyphenyl)-1H-indazol-3-yl)methoxy)phenyl)acetate (238b) (220 mg, 68.8% yield) as a white solid; MS (ES+): 529.20 (M+1):

### Step-3: Preparation of ethyl 2-(2-((5-(3-carbamimidoyl-2-methoxyphenyl)-1-cyclobutyl-1H-indazol-3-yl)methoxy)phenyl)acetate (238c)

Compound 238c was prepared according to the procedure reported in step-1 of scheme-24 from ethyl 2-(2-((1-cyclobutyl-5-(3-(N-hydroxycarbamimidoyl)-2-methoxyphenyl)-1H-indazol-3-yl)methoxy)phenyl)acetate (238b) (220 mg, 0.416 mmol) in EtOH (10 mL) using AcOH (0.024 mL, 0.416 mmol). Raney Nickel (0.416 mmol) and hydrogen (1 atm) to afford after workup and purification using method-AI, ethyl 2-(2-((5-(3-carbamimidoyl-2-methoxyphenyl)-1-cyclobutyl-1H-indazol-3-yl)methoxy)phenyl)acetate (238c) (180 mg, 84% yield) as a colorless oil; MS (ES+): 513.20 (M+1).

### Step-4: Preparation of 2-(2-((5-(3-carbamimidoyl-2-methoxyphenyl)-1-cyclobutyl-1H-indazol-3-yl)methoxy)phenyl)acetic acid (238d)

Compound 238d was prepared according to the procedure reported step-2 of scheme-1 from ethyl 2-(2-((5-(3-carbamimidoyl-2-methoxyphenyl)-1-cyclobutyl-1H-indazol-3-yl)methoxy)phenyl)acetate (238c) (180 mg, 0.351 mmol) in MeOH/THF (6 mL, 1:1) using a solution of LiOH·H₂O (88 mg, 2.107 mmol) in water (2 mL) to afford after workup and purification using method-G, 2-(2-((5-(3-carbamimidoyl-2-methoxyphenyl)-1-cyclobutyl-1H-indazol-3-yl)methoxy)phenyl)acetic acid (238d) (105 mg, 61.7% yield) HCl salt as a white solid; ¹H NMR (300 MHz, DMSO-d₆) δ 12.09 (s, 1H, D₂O exchangeable), 9.41 (s, 4H, D₂O exchangeable), 8.00-7.93 (m, 1H), 7.83 (d, 1H), 7.72-7.59 (m, 2H), 7.53 (dd, J=7.7, 1.7 Hz, 1H), 7.39 (t, J=7.6 Hz, 1H), 7.31-7.22 (m, 2H), 7.22-7.16 (m, 1H), 6.97-6.83 (m, 1H), 5.47 (s, 2H), 5.41-5.26 (m, 1H), 3.52 (s, 2H), 3.37 (s, 3H), 2.77-2.55 (m, 2H), 2.50-2.43 (m, 2H), 1.98-1.79 (m, 2H); MS (ES+): 485.20 (M+1); (ES−): 483.10 (M−1).

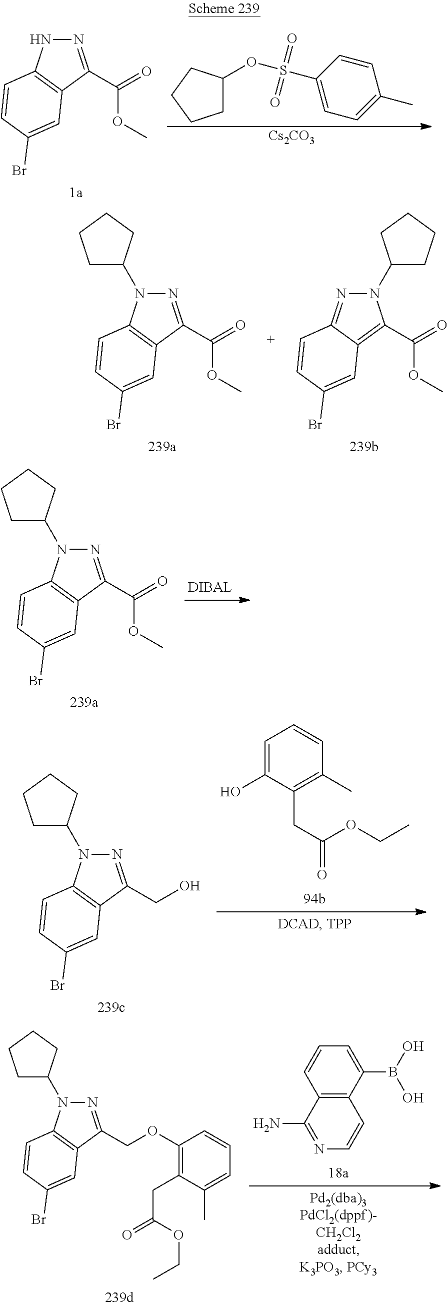

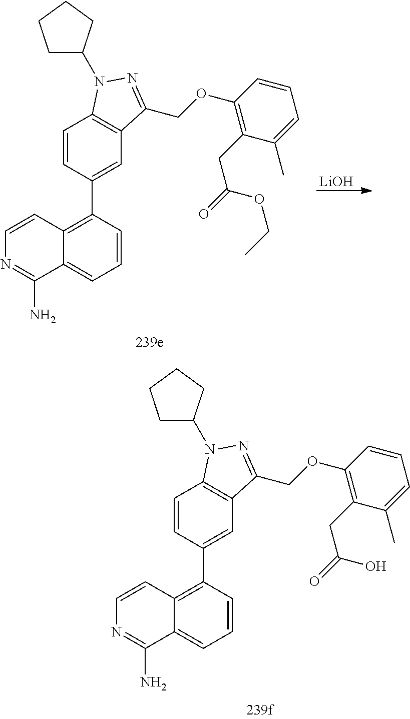

### Preparation of 2-(2-((5-(1-aminoisoquinolin-5-yl)-1-cyclopentyl-1H-indazol-3-yl)methoxy)-6-methylphenyl)acetic acid (239f)

### Step-1: Preparation of methyl 5-bromo-1-cyclopentyl-1H-indazole-3-carboxylate (239a) and methyl 5-bromo-2-cyclopentyl-2H-indazole-3-carboxylate (239b)

Compounds 239a and 239b were prepared according to the procedure reported in step-2 of scheme-86 from methyl 5-bromo-1H-indazole-3-carboxylate (1a) (2.502 g, 9.81 mmol) in DMF (40 mL) using Cs₂CO₃ (6.39 g, 19.62 mmol) and cyclopentyl 4-methylbenzenesulfonate (3.3 g, 13.73 mmol; CAS #3558-06-3) to afford after workup and purification using method-AO, methyl 5-bromo-1-cyclopentyl-1H-indazole-3-carboxylate (239a) (1.85 g, 58.4% yield) as a yellow oil; ¹H NMR (300 MHz, DMSO-d₆) δ 8.23-8.16 (m, 1H), 7.92-7.84 (m, 1H), 7.62 (dd, J=9.0, 1.9 Hz, 1H), 5.40-5.21 (m, 1H), 3.92 (s, 3H), 2.25-2.11 (m, 2H), 2.07-1.98 (m, 2H), 1.93-1.81 (m, 2H), 1.78-1.61 (m, 2H); MS (ES+): 323.00 & 325.00 (M+1); 345.00 & 347.00 (M+Na) and methyl 5-bromo-2-cyclopentyl-2H-indazole-3-carboxylate (239b) (0.62 g, 1.918 mmol, 19.56% yield) as a yellow solid; ¹H NMR (300 MHz, DMSO-d₆) δ 8.16-8.10 (m, 1H), 7.82-7.74 (m, 1H), 7.51-7.42 (m, 1H), 6.02-5.89 (m, 1H), 3.98 (s, 3H), 2.23-2.16 (m, 2H), 2.13-2.05 (m, 2H), 1.91-1.87 (m, 2H), 1.73-1.68 (m, 2H); MS (ES+): 323.00 & 325.00 (M+1).

### Step-2: Preparation of (5-bromo-1-cyclopentyl-1H-indazol-3-yl)methanol (239c)

Compound 239c was prepared according to the procedure reported in step-1 of scheme-2 from methyl 5-bromo-1-cyclopentyl-1H-indazole-3-carboxylate (239a) (1.85 g, 5.72 mmol) in DCM (100 mL) using DIBAL (1M solution in DCM, 14.31 mL, 14.31 mmol) to afford after workup and purification using method-F, (5-bromo-1-cyclopentyl-1H-indazol-3-yl)methanol (239c) (1.23 g, 72.8% yield) as a light yellow oil; ¹H NMR (300 MHz, DMSO-d₆) δ 8.08-7.99 (m, 1H), 7.68-7.61 (m, 1H), 7.47 (dd, J=8.9, 1.9 Hz, 1H), 5.31 (s, 1H), 5.16-5.03 (m, 1H), 4.75 (s, 2H), 2.16-2.03 (m, 2H), 2.03-1.90 (m, 2H), 1.90-1.76 (m, 2H), 1.76-1.57 (m, 2H); MS (ES+): 295.00 & 297.05 (M+1).

### Step-3: Preparation of ethyl 2-(2-((5-bromo-1-cyclopentyl-1H-indazol-3-yl)methoxy)-6-methylphenyl)acetate (239d)

Compound 239d was prepared according to the procedure reported in step-2 of scheme-2 from (5-bromo-1-cyclopentyl-1H-indazol-3-yl)methanol (239c) (1 g, 3.39 mmol) in DCM (20 mL) using ethyl 2-(2-hydroxy-6-methylphenyl)acetate (94b) (0.691 g, 3.56 mmol), PPh₃ (1.066 g, 4.07 mmol) and a solution of DCAD (1.493 g, 4.07 mmol) in DCM (20 mL) to afford after workup and purification using method-N, ethyl 2-(2-((5-bromo-1-cyclopentyl-1H-indazol-3-yl)methoxy)-6-methylphenyl)acetate (239d) (0.7 g, 1.485 mmol, 43.8% yield) as a white oil; ¹H NMR (300 MHz, DMSO-d₆) δ 7.96-7.88 (m, 1H), 7.76-7.68 (m, 1H), 7.51 (dd, J=9.0, 1.8 Hz, 1H), 7.19-7.04 (m, 2H), 6.82 (d, J=7.3 Hz, 1H), 5.35 (s, 2H), 5.24-5.06 (m, 1H), 3.96 (q, J=7.1 Hz, 2H), 3.58 (s, 2H), 2.20 (s, 3H), 2.16-2.06 (m, 2H), 2.06-1.93 (m, 2H), 1.93-1.79 (m, 2H), 1.76-1.63 (m, 2H), 0.99 (t, J=7.1 Hz, 3H); MS (ES+): 471.10 & 473.10 (M+1).

### Step-4: Preparation of ethyl 2-(2-((5-(1-aminoisoquinolin-5-yl)-1-cyclopentyl-1H-indazol-3-yl)methoxy)-6-methylphenyl)acetate (239e)

Compound 239e was prepared according to the procedure reported in step-5 of scheme-1 from ethyl 2-(2-((5-bromo-1-cyclopentyl-1H-indazol-3-yl)methoxy)-6-methylphenyl)acetate (239d) (350 mg, 0.742 mmol) in dioxane/THF (6 mL) using (1-aminoisoquinolin-5-yl)boronic acid (18a) (209 mg, 1.114 mmol), K₃PO₄ (2 M aqueous solution, 1.485 mL, 2.97 mmol), PCy₃ (41.6 mg, 0.148 mmol), PdCl₂(dppf)-CH₂Cl₂ adduct (60.6 mg, 0.074 mmol) and Pd₂(dba)₃ (68.0 mg, 0.074 mmol) to afford after workup and purification using method-Y, ethyl 2-(2-((5-(1-aminoisoquinolin-5-yl)-1-cyclopentyl-1H-indazol-3-yl)methoxy)-6-methylphenyl)acetate (239e) (200 mg, 50.4% yield) as a clear oil; MS (ES+): 535.30 (M+1).

### Step-5: Preparation of 2-(2-((5-(1-aminoisoquinolin-5-yl)-1-cyclopentyl-1H-indazol-3-yl)methoxy)-6-methylphenyl)acetic acid (239f)

Compound 239f was prepared according to the procedure reported in step-2 of scheme-1 from ethyl 2-(2-((5-(1-aminoisoquinolin-5-yl)-1-cyclopentyl-1H-indazol-3-yl)methoxy)-6-methylphenyl)acetate (239e) (200 mg, 0.374 mmol) in MeOH/THF (6 mL, 1:1) using a solution of LiOH·H₂O (94 mg, 2.244 mmol) in water (2 mL) to afford after workup and purification using method-G, 2-(2-((5-(1-aminoisoquinolin-5-yl)-1-cyclopentyl-1H-indazol-3-yl)methoxy)-6-methylphenyl)acetic acid (239f) (85 mg, 44.9% yield) HCl salt as a white solid; ¹H NMR (300 MHz, DMSO-d₆) δ 13.53 (s, 1H, D₂O exchangeable), 12.05 (s, 1H, D₂O exchangeable), 9.29 (s, 2H, DO exchangeable), 8.66 (d, J=8.2 Hz, 1H), 7.98-7.77 (m, 4H), 7.64 (d, J=7.3 Hz, 1H), 7.46 (dd, J=8.6, 1.6 Hz, 1H), 7.17-7.06 (m, 2H), 6.97 (d, J=7.2 Hz, 1H), 6.83-6.71 (m, 1H), 5.41 (s, 2H), 5.25 (m, J=7.1 Hz, 1H), 3.54 (s, 2H), 2.24-2.11 (m, 5H), 2.11-1.98 (m, 2H), 1.98-1.82 (m, 2H), 1.82-1.63 (m, 2H); MS (ES+): 507.25 (M+1); (ES−): 505.10 (M−1).

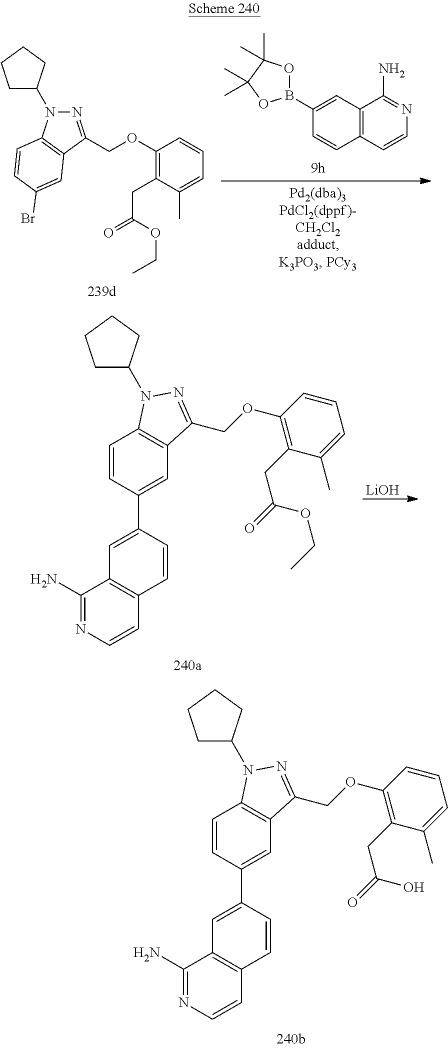

### Preparation of 2-(2-((5-(1-aminoisoquinolin-7-yl)-1-cyclopentyl-1H-indazol-3-yl)methoxy)-6-methylphenyl)acetic acid (240b)

### Step-1: Preparation of ethyl 2-(2-((5-(1-aminoisoquinolin-7-yl)-1-cyclopentyl-1H-indazol-3-yl)methoxy)-6-methylphenyl)acetate (240a)

Compound 240a was prepared according to the procedure reported in step-5 of scheme-1 from ethyl 2-(2-((5-bromo-1-cyclopentyl-1H-indazol-3-yl)methoxy)-6-methylphenyl)acetate (239d) (350 mg, 0.742 mmol) in dioxane/THF (6 mL, 1:1) using 7-(4,4,5,5-tetramethyl-1,3,2-˜ 509-dioxaborolan-2-yl)isoquinolin-1-amine (9h) (301 mg, 1.114 mmol), K₃PO₄ (2 M aqueous solution, 1.485 mL, 2.97 mmol), PCy₃ (41.6 mg, 0.148 mmol), PdCl₂(dppf)-CH₂Cl₂ adduct (60.6 mg, 0.074 mmol) and Pd₂(dba)₃ (68.0 mg, 0.074 mmol) to afford after workup and purification using method-Y, ethyl 2-(2-((5-(1-aminoisoquinolin-7-yl)-1-cyclopentyl-1H-indazol-3-yl)methoxy)-6-methylphenyl)acetate (240a) (250 mg, 63.0% yield) as a clear oil; MS (ES+): 535.25 (M+1).

### Step-2: Preparation of 2-(2-((5-(1-aminoisoquinolin-7-yl)-1-cyclopentyl-1H-indazol-3-yl)methoxy)-6-methylphenyl)acetic acid (240b)

Compound 240b was prepared according to the procedure reported in step-6 of scheme-1 from ethyl 2-(2-((5-(1-aminoisoquinolin-7-yl)-1-cyclopentyl-1H-indazol-3-yl)methoxy)-6-methylphenyl)acetate (240a) (250 mg, 0.468 mmol) in MeOH/THF (6 mL, 1:1) using a solution of LiOH·H₂O (118 mg, 2.81 mmol) in water (2 mL) to afford after workup and purification using method-G, 2-(2-((5-(1-aminoisoquinolin-7-yl)-1-cyclopentyl-1H-indazol-3-yl)methoxy)-6-methylphenyl)acetic acid (240b) (115 mg, 48.5% yield) HCl salt as a white solid; ¹H NMR (300 MHz, DMSO-d₆) δ 9.28 (s, 2H, D₂O exchangeable), 8.98 (d, J=1.8 Hz, 1H), 8.37 (dd, J=8.5, 1.6 Hz, 1H), 8.29 (d, J=1.6 Hz, 1H), 8.07-7.96 (m, 2H), 7.89 (d, J=8.9 Hz, 1H), 7.69 (d, J=6.9 Hz, 1H), 7.25 (d, J=7.0 Hz, 1H), 7.14 (d, J=4.5 Hz, 2H), 6.87-6.74 (m, 1H), 5.45 (s, 2H), 5.30-5.16 (m, 1H), 3.59 (s, 2H), 2.25-2.11 (m, 5H), 2.11-1.97 (m, 2H), 1.97-1.83 (m, 2H), 1.81-1.64 (m, 2H); MS (ES+): 507.20 (M+1); (ES−): 505.20 (M−1).

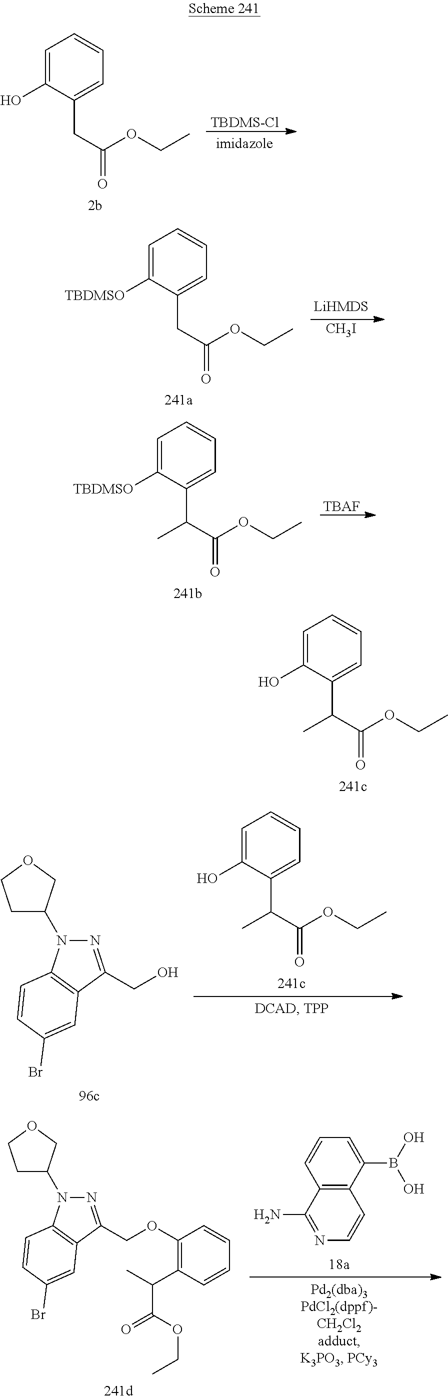

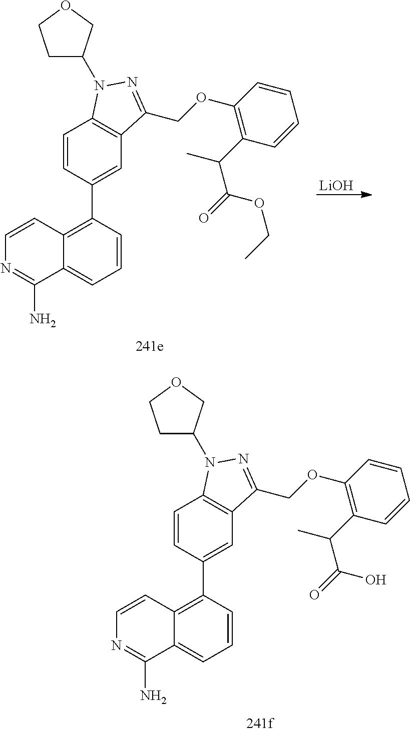 [SMILES: CCOC(=O)C(C)c1ccccc1OCc1nn(C2CCOC2)c2ccc(-c3cccc4c(N)nccc34)cc12 ; "LiOH" ; CC(C(=O)O)c1ccccc1OCc1nn(C2CCOC2)c2ccc(-c3cccc4c(N)nccc34)cc12]

### Preparation of 2-(2-((5-(1-aminoisoquinolin-5-yl)-1-(tetrahydrofuran-3-yl)-1H-indazol-3-yl)methoxy)phenyl)propanoic acid (241f)

### Step-1: Preparation of ethyl 2-(2-(tert-butyldimethylsilyloxy)phenyl)acetate (241a)

To a solution of ethyl 2-(2-hydroxyphenyl)acetate (2b) (3 g, 16.65 mmol) in DCM (50 mL) was added imidazole (2.267 g, 33.3 mmol) and TBDMS-Cl (3.76 g, 24.97 mmol) at 0° C. and the mixture was stirred at RT for 18 h. The reaction mixture was concentrated in vacuo and the residue obtained was dissolved in EtOAc. The organic layer was washed with water, brine, dried, filtered and concentrated in vacuo. The residue obtained was purified using method-BB to give ethyl 2-(2-(tert-butyldimethylsilyloxy)phenyl)acetate (241a) (4 g, 82% yield) as a clear oil; ¹H NMR (300 MHz, DMSO-d₆) δ 7.23-7.11 (m, 2H), 6.94-6.79 (m, 2H), 4.04 (q, J=7.1 Hz, 2H), 3.57 (s, 2H), 1.16 (t, J=7.1 Hz, 3H), 0.95 (s, 9H), 0.20 (s, 6H).

### Step-2: Preparation of ethyl 2-(2-(tert-butyldimethylsilyloxy)phenyl)propanoate (241b)

To a solution of ethyl 2-(2-(tert-butyldimethylsilyloxy)phenyl)acetate (241a) (500 mg, 1.698 mmol) in THF (15 mL) at −78° C. was added LiHMDS (1N in THF, 1.868 mL, 1.868 mmol) and stirred for 30 min. The reaction mixture was treated with a solution of iodomethane (241 mg, 1.698 mmol) in THF (3 mL), stirred at −78° C. for 1 h and warmed to 0° C. over a period of 1 h. The reaction was quenched with 1N HCl, concentrated in vacuum to remove THE and extracted with EtOAc (3×). The combined organic layers were washed with water, brine, dried, filtered and concentrated in vacuo. The residue obtained was purified using method-J to give ethyl 2-(2-(tert-butyldimethylsilyloxy)phenyl)propanoate (241b) (150 mg, 28.6% yield) as a clear oil; MS (ES+): 309.20 (M+1).

### Step-3: Preparation of ethyl 2-(2-hydroxyphenyl)propanoate (241c)

To a solution of ethyl 2-(2-(tert-butyldimethylsilyloxy)phenyl)propanoate (241b) (150 mg, 0.486 mmol) in THF (10 mL) at 0° C. was added N,N-dibutyl-N-propylbutan-1-aminium fluoride (TBAF, 0.608 mL, 0.608 mmol), stirred at 0° C. for 1 h and concentrated in vacuum. The residue was taken up with EtOAc, washed with water, brine, dried, filtered and concentrated in vacuo. The residue obtained was purified using method-J to give ethyl 2-(2-hydroxyphenyl)propanoate (241c) (80 mg, 85% yield) as a white oil; ¹H NMR (300 MHZ, DMSO-d₆) δ 9.51 (s, 1H), 7.14-6.95 (m, 2H), 6.85-6.67 (m, 2H), 4.04 (qd, J=7.1, 1.4 Hz, 2H), 3.91 (q, J=7.1 Hz, 1H), 1.31 (d, J=7.1 Hz, 3H), 1.12 (t, J=7.1 Hz, 3H); MS (ES+): 195.10 (M+1).

### Step-4: Preparation of ethyl 2-(2-((5-bromo-1-(tetrahydrofuran-3-yl)-1H-indazol-3-yl)methoxy)phenyl)propanoate (241d)

Compound 241d was prepared according to the procedure reported in step-2 of scheme-2 from (5-bromo-1-(tetrahydrofuran-3-yl)-1H-indazol-3-yl)methanol (96c) (500 mg, 1.683 mmol) in DCM (20 mL) using ethyl 2-(2-hydroxyphenyl)propanoate (241c) (360 mg, 1.851 mmol), PPh₃ (530 mg, 2.019 mmol) and a solution of DCAD (741 mg, 2.019 mmol) in DCM (20 mL) to afford after workup and purification using method-X, ethyl 2-(2-((5-bromo-1-(tetrahydrofuran-3-yl)-1H-indazol-3-yl)methoxy)phenyl)propanoate (241d) (580 mg, 72.8% yield) as a white solid. ¹H NMR (300 MHz, DMSO-d₆) δ 8.00 (d, J=1.8 Hz, 1H), 7.76 (d, J=9.0 Hz, 1H), 7.56 (dd, J=9.0, 1.8 Hz, 1H), 7.27-7.23 (m, 2H), 7.20-7.14 (m, 1H), 7.00-6.89 (m, 1H), 5.60-5.45 (m, 1H), 5.43-5.37 (m, 2H), 4.16-3.99 (m, 2H), 3.99-3.79 (m, 5H), 2.48-2.37 (m, 1H), 2.37-2.21 (m, 1H), 1.29 (d, J=7.2 Hz, 3H), 0.95 (t, J=7.1 Hz, 3H); MS (ES+): 473.05 & 475.10 (M+1); 495.05 & 497.10 (M+Na); (ES−): 471.00 & 473. (M−1).

### Step-5: Preparation of ethyl 2-(2-((5-(1-aminoisoquinolin-5-yl)-1-(tetrahydrofuran-3-yl)-1H-indazol-3-yl)methoxy)phenyl)propanoate (241e)

Compound 241e was prepared according to the procedure reported in step-5 of scheme-1 from ethyl 2-(2-((5-bromo-1-(tetrahydrofuran-3-yl)-1H-indazol-3-yl)methoxy)phenyl)propanoate (241d) (290 mg, 0.613 mmol) in dioxane/THF (6 mL, 1:1) using (1-aminoisoquinolin-5-yl)boronic acid (18a) (173 mg, 0.919 mmol), K₃PO₄ (2 M aqueous solution, 1.225 mL, 2.451 mmol), PCy₃ (34.4 mg, 0.123 mmol), PdCl₂(dppf)-CH₂Cl₂ adduct (50.0 mg, 0.061 mmol) and Pd₂(dba)₃ (56.1 mg, 0.061 mmol) to afford after workup and purification using method-AP, ethyl 2-(2-((5-(1-aminoisoquinolin-5-yl)-1-(tetrahydrofuran-3-yl)-1H-indazol-3-yl)methoxy)phenyl)propanoate (241e) (180 mg, 54.8% yield) as a clear oil; MS (ES+): 537.30 (M+1).

### Step-6: Preparation of 2-(2-((5-(1-aminoisoquinolin-5-yl)-1-(tetrahydrofuran-3-yl)-1H-indazol-3-yl)methoxy)phenyl)propanoic acid (241f)

Compound 241f was prepared according to the procedure reported in step-2 of scheme-1 from ethyl 2-(2-((5-(1-aminoisoquinolin-5-yl)-1-(tetrahydrofuran-3-yl)-1H-indazol-3-yl)methoxy)phenyl)propanoate (241e) (180 mg, 0.335 mmol) in MeOH/THF (6 mL, 1:1) using a solution of LiOH·H₂O (84 mg, 2.013 mmol) in water (2 mL) to afford after workup and purification using method-G, 2-(2-((5-(1-aminoisoquinolin-5-yl)-1-(tetrahydrofuran-3-yl)-1H-indazol-3-yl)methoxy)phenyl)propanoic acid (241f) (80 mg, 46.9% yield) HCl salt as a white solid; ¹H NMR (300 MHz, DMSO-d₆) δ 13.46 (s, 1H, D₂O exchangeable), 12.00 (s, 1H, D₂O exchangeable), 9.24 (s, 2H, D₂O exchangeable), 8.65 (d, J=8.2 Hz, 1H), 7.95-7.89 (m, 3H), 7.89-7.79 (m, 1H), 7.61 (d, J=7.2 Hz, 1H), 7.52-7.46 (m, 1H), 7.30-7.18 (m, 2H), 7.18-7.11 (m, 1H), 6.97-6.87 (m, 2H), 5.67-5.54 (m, 1H), 5.48 (s, 2H), 4.24-4.05 (m, 2H), 4.05-3.84 (m, 3H), 2.49-2.45 (m, 1H), 2.45-2.31 (m, 1H), 1.24-1.08 (m, 3H); MS (ES+): 509.20 (M+1); (ES−): 507.15 (M−1).

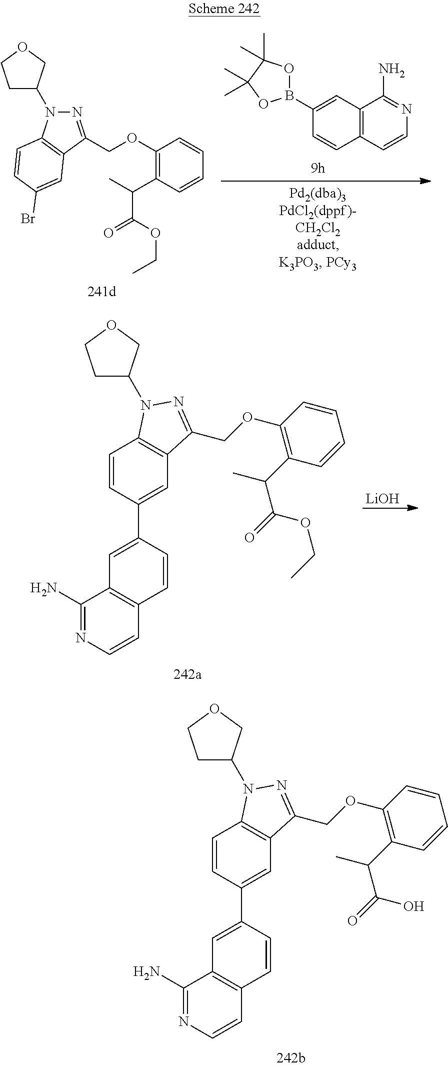

### Preparation of 2-(2-((5-(1-aminoisoquinolin-7-yl)-1-(tetrahydrofuran-3-yl)-1H-indazol-3-yl)methoxy)phenyl)propanoic acid (242b)

### Step-1: Preparation of ethyl 2-(2-((5-(1-aminoisoquinolin-7-yl)-1-(tetrahydrofuran-3-yl)-1H-indazol-3-yl)methoxy)phenyl)propanoate (242a)

Compound 242a was prepared according to the procedure reported in step-5 of scheme-1 from ethyl 2-(2-((5-bromo-1-(tetrahydrofuran-3-yl)-1H-indazol-3-yl)methoxy)phenyl)propanoate (241d) (290 mg, 0.613 mmol) in dioxane/THF (6 mL, 1:1) using 7-(4,4,5,5-tetramethyl-1,3,2-dioxaborolan-2-yl)isoquinolin-1-amine (9h) (248 mg, 0.919 mmol), K₃PO₄ (2 M aqueous solution, 1.225 mL, 2.451 mmol), PCy₃ (34.4 mg, 0.123 mmol), PdCl₂(dppf)-CH₂Cl₂ adduct (50 mg, 0.061 mmol) and Pd₂(dba)₃ (56.1 mg, 0.061 mmol) and Pd₂(dba)₃ (56.1 mg, 0.061 mmol) to afford after workup and purification using method-T, ethyl 2-(2-((5-(1-aminoisoquinolin-7-yl)-1-(tetrahydrofuran-3-yl)-1H-indazol-3-yl)methoxy)phenyl)propanoate (242a) (180 mg, 54.8% yield) as a clear oil; MS (ES+): 537.30 (M+1).

### Step-2: Preparation of 2-(2-((5-(1-aminoisoquinolin-7-yl)-1-(tetrahydrofuran-3-yl)-1H-indazol-3-yl)methoxy)phenyl)propanoic acid (242b)

Compound 242b was prepared according to the procedure reported in step-2 of scheme-1 from ethyl 2-(2-((5-(1-aminoisoquinolin-7-yl)-1-(tetrahydrofuran-3-yl)-1H-indazol-3-yl)methoxy)phenyl)propanoate (242a) (180 mg, 0.335 mmol) in MeOH/THF (6 mL, 1:1) using a solution of LiOH·H₂O (84 mg, 2.013 mmol) in water (2 mL) to afford after workup and purification using method-G, 2-(2-((5-(1-aminoisoquinolin-7-yl)-1-(tetrahydrofuran-3-yl)-1H-indazol-3-yl)methoxy)phenyl)propanoic acid (242b) (75 mg, 44.0% yield) HCl salt as a white solid; ¹H NMR (300 MHz, DMSO-d₆) δ 13.40 (s, 1H, D₂O exchangeable), 12.09 (s, 1H, D₂O exchangeable), 9.21 (s, 2H, D₂O exchangeable), 8.99 (d, 1H), 8.46-8.31 (m, 2H), 8.10-7.99 (m, 2H), 7.99-7.88 (m, 1H), 7.69 (d, J=6.9 Hz, 1H), 7.33-7.23 (m, 3H), 7.23-7.13 (m, 1H), 7.00-6.88 (m, 1H), 5.65-5.54 (m, 1H), 5.52 (s, 2H), 4.23-4.06 (m, 2H), 4.06-3.84 (m, 3H), 2.49-2.43 (m, 1H), 2.42-2.29 (m, 1H), 1.22 (dd, J=7.2, 1.4 Hz, 3H); MS (ES+): 509.25 (M+1); MS (ES−): 507.20 (M−1).

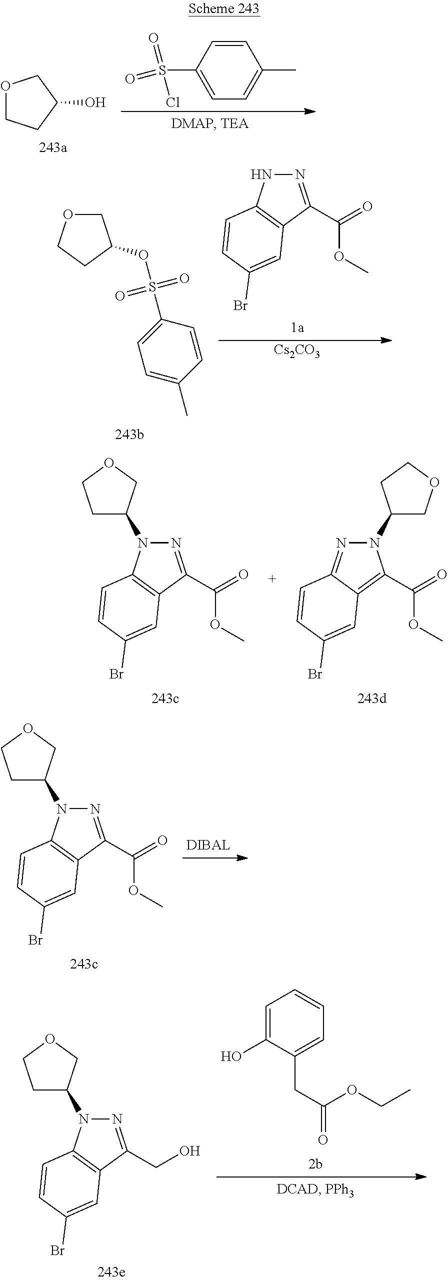

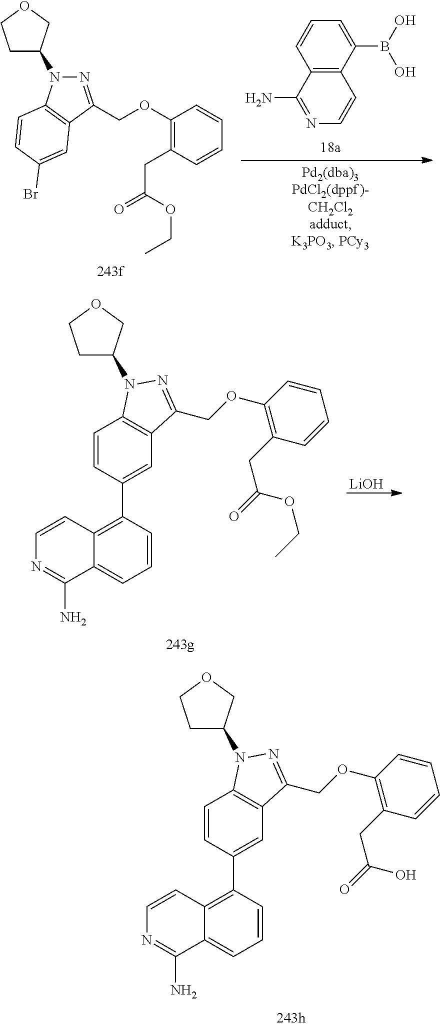 [SMILES: CCOC(=O)Cc1ccccc1OCc1nn([C@H]2CCOC2)c2ccc(Br)cc12 ; Nc1nccc2c(B(O)O)cccc12 ; CCOC(=O)Cc1ccccc1OCc1nn([C@H]2CCOC2)c2ccc(-c3cccc4c(N)nccc34)cc12 ; "LiOH" ; Nc1nccc2c(-c3ccc4c(c3)c(COc3ccccc3CC(=O)O)nn4[C@H]3CCOC3)cccc12]

### Preparation of (S)-2-(2-((5-(1-aminoisoquinolin-5-yl)-1-(tetrahydrofuran-3-yl)-1H-indazol-3-yl)methoxy)phenyl)acetic acid (243h)

### Step-1: Preparation of (R)-tetrahydrofuran-3-yl 4-methylbenzenesulfonate (243b)

Compound 243b was prepared according to the procedure reported in step-1 of scheme-86 from (R)-tetrahydrofuran-3-ol (243a) (8 g, 91 mmol; CAS #86087-24-3) in anhydrous DCM (200 mL) using DMAP (0.555 g, 4.54 mmol), TEA (18.98 mL, 136 mmol) and p-toluenesulfonylchloride (34.6 g, 182 mmol) to afford after workup and purification using method-AJ, to afford after workup and purification using method-AO, (R)-tetrahydrofuran-3-yl 4-methylbenzenesulfonate (243b) (16.5 g, 75.0% yield) as a clear oil; ¹H NMR (300 MHz, DMSO-d₆) δ 7.90-7.72 (m, 2H), 7.55-7.41 (m, 2H), 5.18-5.03 (m, 1H), 3.81-3.60 (m, 4H), 2.42 (s, 3H), 2.14-2.00 (m, 1H), 1.95-1.79 (m, 1H); MS (ES+): 265.00 (M+Na).

### Step-2: Preparation of (S)-methyl 5-bromo-1-(tetrahydrofuran-3-yl)-1H-indazole-3-carboxylate (243c) and (S)-methyl 5-bromo-2-(tetrahydrofuran-3-yl)-2H-indazole-3-carboxylate (243d)

Compounds 243c and 243d were prepared according to the procedure reported in step-2 of scheme-86 from methyl 5-bromo-1H-indazole-3-carboxylate (1a) (15.70 g, 61.5 mmol) in DMF (100 mL) using Cs₂CO₃ (40.1 g, 123 mmol) and (R)-tetrahydrofuran-3-yl 4-methylbenzenesulfonate (243b) (16.4 g, 67.7 mmol) to afford after workup and purification using method-AO. (S)-methyl 5-bromo-1-(tetrahydrofuran-3-yl)-1H-indazole-3-carboxylate (243c) (8.6 g, 43.0% yield) as a white solid; ¹H NMR (300 MHz, DMSO-d₆) δ 8.20 (dd, J=1.9, 0.7 Hz, 1H), 7.89 (dd, J=9.0, 0.7 Hz, 1H), 7.65 (dd, J=9.0, 1.9 Hz, 1H), 5.74-5.44 (m, 1H), 4.17-4.00 (m, 2H), 4.00-3.94 (m, 1H), 3.93 (s, 3H), 3.92-3.81 (m, 1H), 2.49-2.26 (m, 2H); MS (ES+): 325.00 & 327.00 (M+1); 347.00 & 349.00 (M+Na) and (S)-methyl 5-bromo-2-(tetrahydrofuran-3-yl)-2H-indazole-3-carboxylate (243d) (3.6 g, 17.99% yield) as a white solid; MS (ES+): 325.00 & 327.00 (M+1); 347.00 & 349.00 (M+Na).

### Step-3: Preparation of (S)-(5-bromo-1-(tetrahydrofuran-3-yl)-1H-indazol-3-yl)methanol (243e)

Compound 243e was prepared according to the procedure reported in step-1 of scheme-2 from (S)-methyl 5-bromo-1-(tetrahydrofuran-3-yl)-1H-indazole-3-carboxylate (243c) (8 g, 24.60 mmol) in DCM (100 mL) using DIBAL (1M solution in DCM, 61.5 mL, 61.5 mmol) to afford after workup and purification using method-F. (S)-(5-bromo-1-(tetrahydrofuran-3-yl)-1H-indazol-3-yl)methanol (243e) (5.42 g, 74.1% yield) as a light yellow oil; ¹H NMR (300 MHz, DMSO-d₆) δ 8.07 (dd, J=1.9, 0.7 Hz, 1H), 7.69 (dd, J=9.0, 0.6 Hz, 1H), 7.51 (dd, J=8.9, 1.9 Hz, 1H), 5.52-5.39 (m, 1H), 5.35 (t, J=5.9 Hz, 1H), 4.75 (d, J=5.9 Hz, 2H), 4.14-3.96 (m, 2H), 3.93-3.78 (m, 2H), 2.47-2.34 (m, 1H), 2.33-2.18 (m, 1H); MS (ES+): 297.00 & 299.00 (M+1); 319.00 & 321.00 (M+Na).

### Step-4: Preparation of (S)-ethyl 2-(2-((5-bromo-1-(tetrahydrofuran-3-yl)-1H-indazol-3-yl)methoxy)phenyl)acetate (243f)

Compound 243f was prepared according to the procedure reported in step-2 of scheme-2 from (S)-(5-bromo-1-(tetrahydrofuran-3-yl)-1H-indazol-3-yl)methanol (243e) (1 g, 3.37 mmol) in DCM (20 mL) using ethyl 2-(2-hydroxyphenyl)acetate (2b) (0.667 g, 3.70 mmol), PPh₃ (1.059 g, 4.04 mmol) and a solution of DCAD (1.483 g, 4.04 mmol) in DCM (20 mL) to afford after workup and purification using method-N, (S)-ethyl 2-(2-((5-bromo-1-(tetrahydrofuran-3-yl)-1H-indazol-3-yl)methoxy)phenyl)acetate (243f) (1.2 g, 78% yield) as a white oil; ¹H NMR (300 MHz, DMSO-d₆) δ 8.00-7.93 (m, 1H), 7.78-7.73 (m, 1H), 7.56 (dd. J=9.0, 1.8 Hz, 1H), 7.46-7.42 (m, 1H), 7.29-7.25 (m, 1H), 7.22-7.18 (m, 1H), 6.98-6.88 (m, 1H), 5.56-5.44 (m, 1H), 5.37 (s, 2H), 4.14-4.05 (m, 2H), 3.96-3.82 (m, 4H), 3.53 (s, 2H), 2.47-2.21 (m, 2H), 0.94 (t, J=7.1 Hz, 3H); MS (ES+): 459.10 & 461.10 (M+1); 481.00 & 483.00 (M+Na); (ES−): 457.00 & 459.00 (M−1).

### Step-5: Preparation of (S)-ethyl 2-(2-((5-(1-aminoisoquinolin-5-yl)-1-(tetrahydrofuran-3-yl)-1H-indazol-3-yl)methoxy)phenyl)acetate (243g)

Compound 243g was prepared according to the procedure reported in step-5 of scheme-1 from (S)-ethyl 2-(2-((5-bromo-1-(tetrahydrofuran-3-yl)-1H-indazol-3-yl)methoxy)phenyl)acetate (243f) (300 mg, 0.653 mmol) in dioxane/THF (6 mL) using (1-aminoisoquinolin-5-yl)boronic acid (18a) (184 mg, 0.980 mmol), K₃PO₄ (2 M aqueous solution, 1.306 mL, 2.61 mmol), PCy₃ (36.6 mg, 0.131 mmol). PdCl₂(dppf)-CH₂Cl₂ adduct (53.3 mg, 0.065 mmol) and Pd₂(dba)₃ (59.8 mg, 0.065 mmol) to afford after workup and purification using method-Y, (S)-ethyl 2-(2-((5-(1-aminoisoquinolin-5-yl)-1-(tetrahydrofuran-3-yl)-1H-indazol-3-yl)methoxy)phenyl)acetate (243g) (200 mg, 58.6% yield) as a clear oil; MS (ES+): 523.20 (M+1).

### Step-6: Preparation of (S)-2-(2-((5-(1-aminoisoquinolin-5-yl)-1-(tetrahydrofuran-3-yl)-1H-indazol-3-yl)methoxy)phenyl)acetic acid (243h)

Compound 243h was prepared according to the procedure reported in step-2 of scheme-1 from (S)-ethyl 2-(2-((5-(1-aminoisoquinolin-5-yl)-1-(tetrahydrofuran-3-yl)-1H-indazol-3-yl)methoxy)phenyl)acetate (243g) (200 mg, 0.383 mmol) in MeOH/THF (6 mL, 1:1) using a solution of LiOH·H₂O (96 mg, 2.296 mmol) in water (2 mL) to afford after workup and purification using method-G. (S)-2-(2-((5-(1-aminoisoquinolin-5-yl)-1-(tetrahydrofuran-3-yl)-1H-indazol-3-yl)methoxy)phenyl)acetic acid (243h) (40 mg, 21.13% yield) HCl salt as a white solid; ¹H NMR (300 MHz, DMSO-d₆) δ 13.30 (s, 1H, D₂O exchangeable), 12.01 (s, 1H, D₂O exchangeable), 9.16 (s, 2H, D₂O exchangeable), 8.62 (d, J=8.3 Hz, 1H), 7.99-7.81 (m, 4H), 7.62 (d, J=7.3 Hz, 1H), 7.50 (dd. J=8.7, 1.6 Hz, 1H), 7.28-7.21 (m, 2H), 7.21-7.14 (m, 1H), 6.98 (d, J=7.2 Hz, 1H), 6.95-6.85 (m, 1H), 5.68-5.52 (m, 1H), 5.45 (s, 2H), 4.21-4.07 (m, 2H), 4.03-3.87 (m, 2H), 3.48 (s, 2H), 2.48-2.23 (m, 2H); MS (ES+): 495.20 (M+1); (ES−): 493.10 (M−1); Analysis calculated for C₂₉H₂₆N₄O₄·1.05HCl·1.5H₂O: C, 62.22; H, 5.41; Cl, 6.65; N, 10.01; Found: C, 62.14; H, 5.13; Cl, 6.99; N, 9.78.

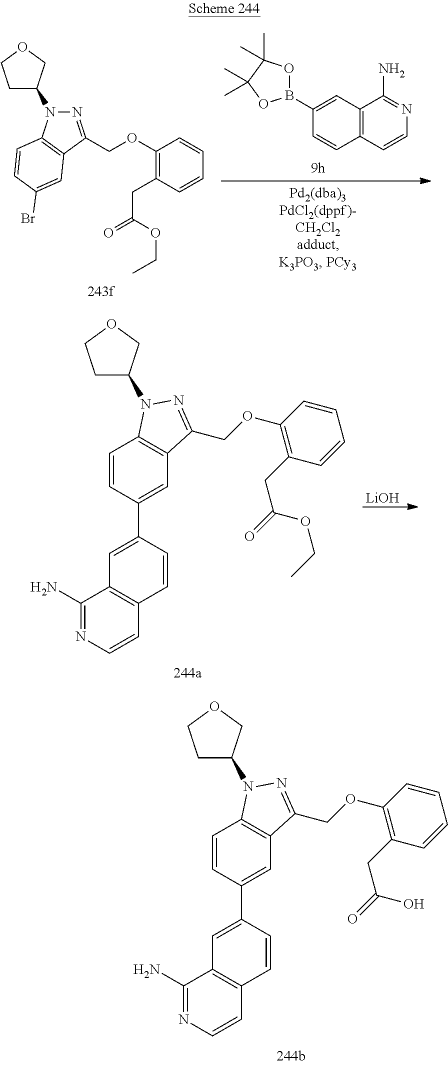

### Preparation of (S)-2-(2-((5-(1-aminoisoquinolin-7-yl)-1-(tetrahydrofuran-3-yl)-1H-indazol-3-yl)methoxy)phenyl)acetic acid (244b)

### Step-1: Preparation of (S)-ethyl 2-(2-((5-(1-aminoisoquinolin-7-yl)-1-(tetrahydrofuran-3-yl)-1H-indazol-3-yl)methoxy)phenyl)acetate (244a)

Compound 244a was prepared according to the procedure reported in step-5 of scheme-1 from (S)-ethyl 2-(2-((5-bromo-1-(tetrahydrofuran-3-yl)-1H-indazol-3-yl)methoxy)phenyl)acetate (243f) (300 mg, 0.653 mmol) in dioxane/THF (6 mL, 1:1) using 7-(4,4,5,5-tetramethyl-1,3,2-dioxaborolan-2-yl)isoquinolin-1-amine (9h) (265 mg, 0.980 mmol), K₃PO₄ (2 M aqueous solution, 1.306 mL, 2.61 mmol), PCy₃ (36.6 mg, 0.131 mmol), PdCl₂(dppf)-CH₂Cl₂ adduct (53.3 mg, 0.065 mmol) and Pd₂(dba)₃ (59.8 mg, 0.065 mmol) to afford after workup and purification using method-Y, (S)-ethyl 2-(2-((5-(1-aminoisoquinolin-7-yl)-1-(tetrahydrofuran-3-yl)-1H-indazol-3-yl)methoxy)phenyl)acetate (244a) (200 mg, 58.6% yield) as a clear oil; MS (ES+): 523.30 (M+1).

### Step-2: Preparation of (S)-2-(2-((5-(1-aminoisoquinolin-7-yl)-1-(tetrahydrofuran-3-yl)-1H-indazol-3-yl)methoxy)phenyl)acetic acid (244b)

Compound 244b was prepared according to the procedure reported in step-2 of scheme-1 from (S)-ethyl 2-(2-((5-(1-aminoisoquinolin-7-yl)-1-(tetrahydrofuran-3-yl)-1H-indazol-3-yl)methoxy)phenyl)acetate (244a) (200 mg, 0.383 mmol) in MeOH/THF (6 mL, 1:1) using a solution of LiOH·H₂O (96 mg, 2.296 mmol) in water (2 mL) to afford after workup and purification using method-G, (S)-2-(2-((5-(1-aminoisoquinolin-7-yl)-1-(tetrahydrofuran-3-yl)-1H-indazol-3-yl)methoxy)phenyl)acetic acid (244b) (65 mg, 34.3% yield) HCl salt as a white solid; ¹H NMR (300 MHz, DMSO-d₆) δ 13.23 (s, 1H, D₂O exchangeable), 12.11 (s, 1H, D₂O exchangeable), 9.11 (s, 2H, D₂O exchangeable), 8.98-8.91 (m, 1H), 8.41 (dd, J=8.5, 1.6 Hz, 1H), 8.32 (d, 1H), 8.09-7.98 (m, 2H), 7.98-7.91 (m, 1H), 7.69 (d, J=6.9 Hz, 1H), 7.32-7.23 (m, 3H), 7.23-7.16 (m, 1H), 6.98-6.87 (m, 1H), 5.65-5.52 (m, 1H), 5.48 (s, 2H), 4.22-4.06 (m, 2H), 4.02-3.84 (m, 2H), 3.53 (s, 2H), 2.48-2.23 (m, 2H); MS (ES+): 495.20 (M+1); (ES−): 493.10 (M−1); Analysis calculated for C₂₉H₂₆N₄O₄·1.1HCl·1.25H₂O: C, 62.52; H, 5.36; Cl, 7.00; N, 10.06; Found: C, 62.74; H, 5.26; Cl, 7.15; N, 10.03.

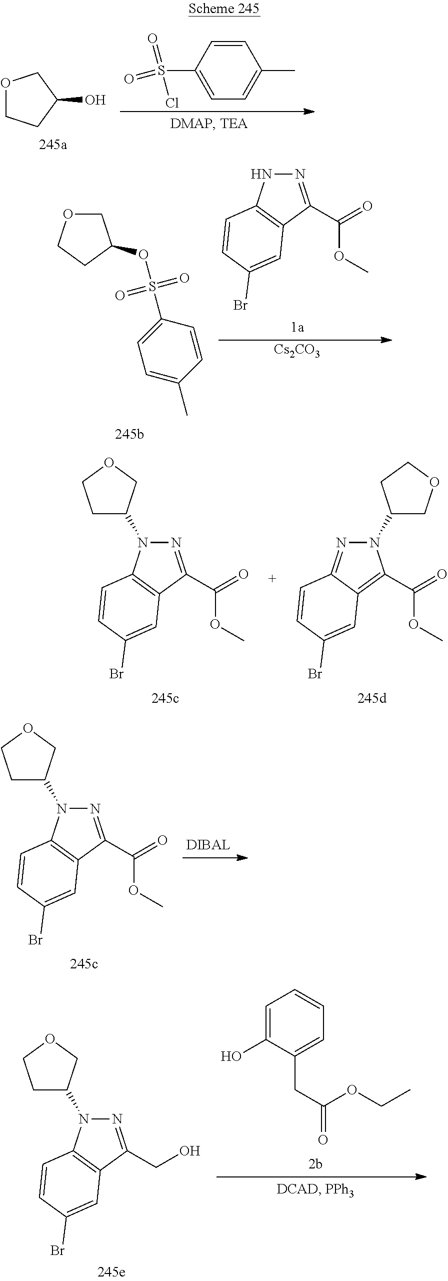

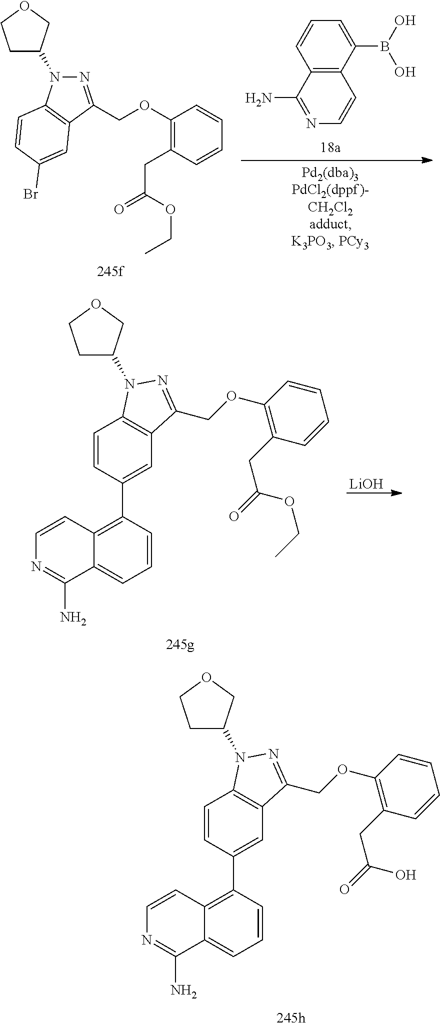

### Preparation of (R)-2-(2-((5-(1-aminoisoquinolin-5-yl)-1-(tetrahydrofuran-3-yl)-1H-indazol-3-yl)methoxy)phenyl)acetic acid (245h)

### Step-1: Preparation of (S)-tetrahydrofuran-3-yl 4-methylbenzenesulfonate (245b)

Compound 245b was prepared according to the procedure reported in step-1 of scheme-86 from (S)-tetrahydrofuran-3-ol (245a) (8 g, 91 mmol; CAS #86087-23-2) in anhydrous DCM (200 mL) using DMAP (0.555 g, 4.54 mmol), TEA (18.98 mL, 136 mmol) and p-toluenesulfonylchloride (34.6 g, 182 mmol) to afford after workup and purification using method-AJ, (S)-tetrahydrofuran-3-yl 4-methylbenzenesulfonate (245b) (15.5 g, 64.0 mmol, 70.5% yield) as a clear oil. ¹H NMR (300 MHz, DMSO-d₆) δ 7.88-7.76 (m, 2H), 7.56-7.41 (m, 2H), 5.20-5.04 (m, 1H), 3.80-3.61 (m, 4H), 2.42 (s, 3H), 2.16-2.00 (m, 1H), 1.94-1.79 (m, 1H); MS (ES+): 265.00 (M+Na).

### Step-2: Preparation of (R)-methyl 5-bromo-1-(tetrahydrofuran-3-yl)-1H-indazole-3-carboxylate (245c) and (R)-methyl 5-bromo-2-(tetrahydrofuran-3-yl)-2H-indazole-3-carboxylate (245d)

Compounds 245c and 245d were prepared according to the procedure reported in step-2 of scheme-86 from methyl 5-bromo-1H-indazole-3-carboxylate (1a) (14.74 g, 57.8 mmol) in DMF (100 mL) using Cs₂CO₃ (37.7 g, 116 mmol) and (S)-tetrahydrofuran-3-yl 4-methylbenzenesulfonate (245b) (15.4 g, 63.6 mmol) to afford after workup and purification using method-AO. (R)-methyl 5-bromo-1-(tetrahydrofuran-3-yl)-1H-indazole-3-carboxylate (245c) (8 g, 42.6% yield) as a white solid; H NMR (300 MHz, DMSO-d₆) δ 8.20 (dd, J=1.9, 0.7 Hz, 1H), 7.90 (dd, J=9.0, 0.7 Hz, 1H), 7.65 (dd, J=9.0, 1.9 Hz, 1H), 5.63 (ddt, J=8.1, 6.6, 3.9 Hz, 1H), 4.18-4.03 (m, 2H), 4.00-3.81 (m, 5H), 2.49-2.25 (m, 2H); MS (ES+): 325.00 & 327.00 (M+1); 347.00 & 349.00 (M+Na) and (R)-methyl 5-bromo-2-(tetrahydrofuran-3-yl)-2H-indazole-3-carboxylate (245d) (3 g, 15.97% yield) as a white solid; MS (ES+): 325.00 & 327.00 (M+1); 347.00 & 349.00 (M+Na).

### Step-3: Preparation of (R)-(5-bromo-1-(tetrahydrofuran-3-yl)-1H-indazol-3-yl)methanol (245e)

Compound 245e was prepared according to the procedure reported in step-1 of scheme-2 from (R)-methyl 5-bromo-1-(tetrahydrofuran-3-yl)-1H-indazole-3-carboxylate (245c) (8 g, 24.60 mmol) in DCM (100 mL) using DIBAL (1M solution in DCM, 61.5 mL, 61.5 mmol) to afford after workup and purification using method-F, (R)-(5-bromo-1-(tetrahydrofuran-3-yl)-1H-indazol-3-yl)methanol (245e) (5.65 g, 77% yield) as a light yellow solid; ¹H NMR (300 MHz, DMSO-d₆) δ 8.07 (dd, J=1.9, 0.7 Hz, 1H), 7.69 (dd, J=9.0, 0.7 Hz, 1H), 7.50 (dd, J=8.9, 1.9 Hz, 1H), 5.51-5.39 (m, 1H), 5.36 (t, J=5.9 Hz, 1H), 4.75 (d, J=5.9 Hz, 2H), 4.12-3.97 (m, 2H), 3.92-3.80 (m, 2H), 2.48-2.34 (m, 1H), 2.34-2.17 (m, 1H); MS (ES+): 297.00 & 299.00 (M+1); 319.00 & 321.00 (M+Na).

### Step-4: Preparation of (R)-ethyl 2-(2-((5-bromo-1-(tetrahydrofuran-3-yl)-1H-indazol-3-yl)methoxy)phenyl)acetate (245f)

Compound 245f was prepared according to the procedure reported in step-2 of scheme-2 from (R)-(5-bromo-1-(tetrahydrofuran-3-yl)-1H-indazol-3-yl)methanol (245e) (1 g, 3.37 mmol) in DCM (20 mL) using ethyl 2-(2-hydroxyphenyl)acetate (2b) (0.667 g, 3.70 mmol), PPh₃ (1.059 g, 4.04 mmol) and a solution of DCAD (1.483 g, 4.04 mmol) in DCM (20 mL) to afford after workup and purification using method-N. (R)-ethyl 2-(2-((5-bromo-1-(tetrahydrofuran-3-yl)-1H-indazol-3-yl)methoxy)phenyl)acetate (245f) (1.2 g, 78% yield) as a white oil; ¹H NMR (300 MHz, DMSO-d₆) δ7.99-7.93 (m, 1H), 7.79-7.72 (m, 1H), 7.56 (dd, J=9.0, 1.9 Hz, 1H), 7.45-7.39 (m, 1H), 7.29-7.25 (m, 1H), 7.23-7.17 (m, 1H), 6.97-6.88 (m, 1H), 5.60-5.42 (m, 1H), 5.37 (s, 2H), 4.07-3.98 (m, 2H), 3.96-3.83 (m, 4H), 3.53 (s, 2H), 2.47-2.23 (m, 2H), 0.94 (t, J=7.1 Hz, 3H); MS (ES+): 459.10 & 461.10 (M+1); 481.00 & 483.10 (M+Na); (ES−): 457.00 & 459.00 (M−1).

### Step-5: Preparation of (R)-ethyl 2-(2-((5-(1-aminoisoquinolin-5-yl)-1-(tetrahydrofuran-3-yl)-1H-indazol-3-yl)methoxy)phenyl)acetate (245g)

Compound 245g was prepared according to the procedure reported in step-5 of scheme-1 from (R)-ethyl 2-(2-((5-bromo-1-(tetrahydrofuran-3-yl)-1H-indazol-3-yl)methoxy)phenyl)acetate (245f) (300 mg, 0.653 mmol) in dioxane/THF (6 mL, 1:1) using (1-aminoisoquinolin-5-yl)boronic acid (18a) (184 mg, 0.980 mmol), K₃PO₄ (2 M aqueous solution, 1.306 mL, 2.61 mmol), PCy₃ (36.6 mg, 0.131 mmol). PdCl₂(dppf)-CH₂Cl₂ adduct (53.3 mg, 0.065 mmol) and Pd₂(dba)₃ (59.8 mg, 0.065 mmol) to afford after workup and purification using method-Y. (R)-ethyl 2-(2-((5-(1-aminoisoquinolin-5-yl)-1-(tetrahydrofuran-3-yl)-1H-indazol-3-yl)methoxy)phenyl)acetate (245g) (200 mg, 58.6% yield) as a clear oil; MS (ES+): 523.20 (M+1).

### Step-6: Preparation of (R)-2-(2-((5-(1-aminoisoquinolin-5-yl)-1-(tetrahydrofuran-3-yl)-1H-indazol-3-yl)methoxy)phenyl)acetic acid (245h)

Compound 245h was prepared according to the procedure reported in step-2 of scheme-1 from (R)-ethyl 2-(2-((5-(1-aminoisoquinolin-5-yl)-1-(tetrahydrofuran-3-yl)-1H-indazol-3-yl)methoxy)phenyl)acetate (245g) (200 mg, 0.383 mmol) in MeOH/THF (6 mL, 1:1) using a solution of LiOH·H₂O (96 mg, 2.296 mmol) in water (2 mL) to afford after workup and purification using method-G. (R)-2-(2-((5-(1-aminoisoquinolin-5-yl)-1-(tetrahydrofuran-3-yl)-1H-indazol-3-yl)methoxy)phenyl)acetic acid (245h) (45 mg, 23.78% yield) HCl salt as a white solid; ¹H NMR (300 MHz, DMSO-d₆) δ 13.16 (s, 1H, D₂O exchangeable), 12.01 (s, 1H, DO exchangeable), 9.09 (s, 2H, D₂O exchangeable), 8.60 (d, J=8.3 Hz, 1H), 8.02-7.80 (m, 4H), 7.62 (d, J=7.2 Hz, 1H), 7.50 (dd. J=8.7, 1.6 Hz, 1H), 7.29-7.22 (m, 2H), 7.19-7.14 (m, 1H), 6.98 (d, J=7.2 Hz, 1H), 6.94-6.86 (m, 1H), 5.67-5.52 (m, 1H), 5.45 (s, 2H), 4.21-4.06 (m, 2H), 4.06-3.86 (m, 2H), 3.47 (s, 2H), 2.48-2.30 (m, 2H); MS (ES+): 495.20 (M+1); (ES−): 493.10 (M−1).

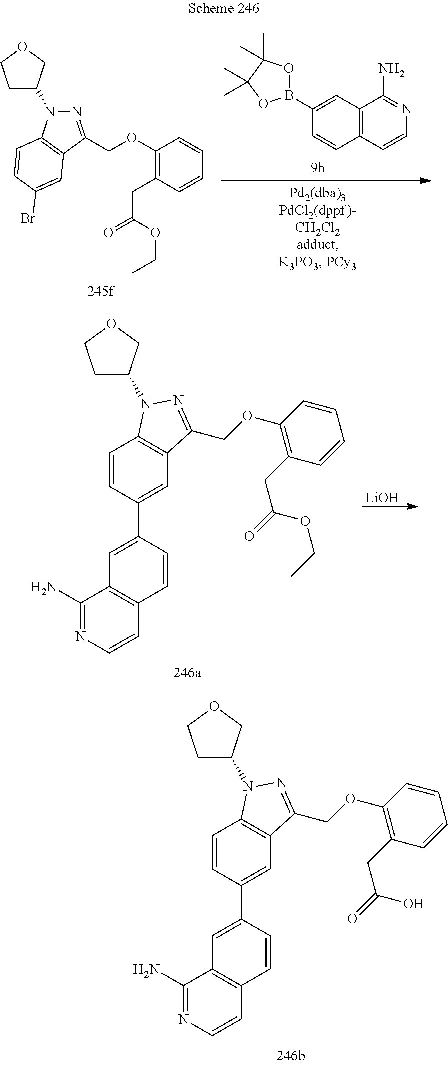

### Preparation of (R)-2-(2-(5-(1-aminoisoquinolin-7-yl)-1-(tetrahydrofuran-3-yl)-1H-indazol-3-yl)methoxy)phenyl)acetic acid (246b)

### Step-1: Preparation of (R)-ethyl 2-(2-((5-(1-aminoisoquinolin-7-yl)-1-(tetrahydrofuran-3-yl)-1H-indazol-3-yl)methoxy)phenyl)acetate (246a)

Compound 246a was prepared according to the procedure reported in step-5 of scheme-1 from (R)-ethyl 2-(2-((5-bromo-1-(tetrahydrofuran-3-yl)-1H-indazol-3-yl)methoxy)phenyl)acetate (245f) (300 mg, 0.653 mmol) in dioxane/THF (6 mL, 1:1) using 7-(4,4,5,5-tetramethyl-1,3,2-dioxaborolan-2-yl)isoquinolin-1-amine (9h) (265 mg, 0.980 mmol), K₃PO₄ (2 M aqueous solution, 1.306 mL, 2.61 mmol), PCy₃ (36.6 mg, 0.131 mmol), PdCl₂(dppf)-CH₂Cl₂ adduct (53.3 mg, 0.065 mmol) and Pd₂(dba)₃ (59.8 mg, 0.065 mmol) to afford after workup and purification using method-Y, (R)-ethyl 2-(2-((5-(1-aminoisoquinolin-7-yl)-1-(tetrahydrofuran-3-yl)-1H-indazol-3-yl)methoxy)phenyl)acetate (246a) (200 mg, 58.6% yield) as a clear oil; MS (ES+): 523.30 (M+1); (ES−): 521.15 (M−1).

### Step-2: Preparation of (R)-2-(2-((5-(1-aminoisoquinolin-7-yl)-1-(tetrahydrofuran-3-yl)-1H-indazol-3-yl)methoxy)phenyl)acetic acid (246b)

Compound 246b was prepared according to the procedure reported in step-2 of scheme-1 from (R)-ethyl 2-(2-((5-(1-aminoisoquinolin-7-yl)-1-(tetrahydrofuran-3-yl)-1H-indazol-3-yl)methoxy)phenyl)acetate (246a) (200 mg, 0.383 mmol) in MeOH/THF (6 mL, 1:1) using a solution of LiOH·H₂O (96 mg, 2.296 mmol) in water (2 mL) to afford after workup and purification using method-G, (R)-2-(2-((5-(1-aminoisoquinolin-7-yl)-1-(tetrahydrofuran-3-yl)-1H-indazol-3-yl)methoxy)phenyl)acetic acid (246b) (55 mg, 29.1% yield) HCl salt as a white solid; ¹H NMR (300 MHz, DMSO-d₆) δ 13.29 (s, 1H, D₂O exchangeable), 12.17 (s, 1H, D₂O exchangeable), 9.17 (s, 2H, D₂O exchangeable), 8.97 (d, 1H), 8.41 (dd, J=8.5, 1.6 Hz, 1H), 8.33 (d, 1H), 8.12-7.99 (m, 2H), 7.99-7.90 (m, 1H), 7.69 (d, J=6.9 Hz, 1H), 7.33-7.16 (m, 4H), 6.98-6.86 (m, 1H), 5.68-5.54 (m, 1H), 5.48 (s, 2H), 4.22-4.05 (m, 2H), 4.05-3.84 (m, 2H), 3.53 (s, 2H), 2.49-2.25 (m, 2H); MS (ES+): 495.20 (M+1); MS (ES−): 493.10 (M−1); Analysis calculated for C₂₉H₂₆N₄O₄·1.1HCl·1.25H₂O: C, 62.52; H, 5.36; Cl, 7.00; N, 10.06; Found: C, 62.64; H, 5.30; Cl, 7.27; N, 10.06.

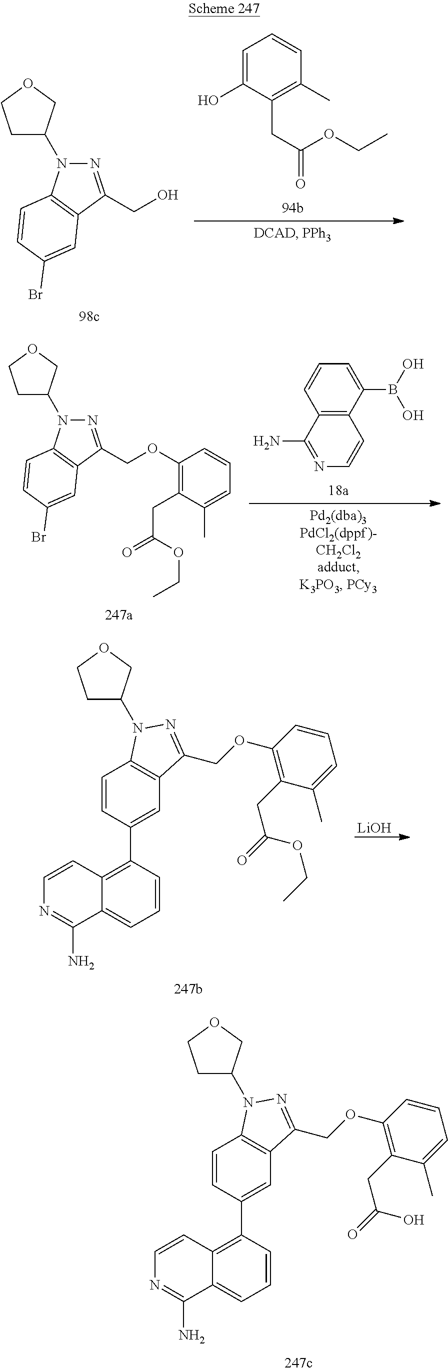

### Preparation of 2-(2-((5-(1-aminoisoquinolin-5-yl)-1-(tetrahydrofuran-3-yl)-1H-indazol-3-yl)methoxy)-6-methylphenyl)acetic acid (247c)

### Step-1: Preparation of ethyl 2-(2-((5-bromo-1-(tetrahydrofuran-3-yl)-1H-indazol-3-yl)methoxy)-6-methylphenyl)acetate (247a)

Compound 247a was prepared according to the procedure reported in step-2 of scheme-2 from (5-bromo-1-(tetrahydrofuran-3-yl)-1H-indazol-3-yl)methanol (96c) (500 mg, 1.683 mmol) in DCM (10 mL) using ethyl 2-(2-hydroxy-6-methylphenyl)acetate (94b) (343 mg, 1.767 mmol) PPh₃ (530 mg, 2.019 mmol) and a solution of DCAD (741 mg, 2.019 mmol) in DCM (10 mL) to afford after workup and purification using method-N, ethyl 2-(2-((5-bromo-1-(tetrahydrofuran-3-yl)-1H-indazol-3-yl)methoxy)-6-methylphenyl)acetate (247a) (500 mg, 62.8% yield) as a white oil; MS (ES+): 473.10 & 475.10 (M+1); 495.05 & 497.05 (M+Na).

### Step-2: Preparation of ethyl 2-(2-((5-(1-aminoisoquinolin-5-yl)-1-(tetrahydrofuran-3-yl)-1H-indazol-3-yl)methoxy)-6-methylphenyl)acetate (247b)

Compound 247b was prepared according to the procedure reported in step-5 of scheme-1 from ethyl 2-(2-((5-bromo-1-(tetrahydrofuran-3-yl)-1H-indazol-3-yl)methoxy)-6-methylphenyl)acetate (247a) (250 mg, 0.528 mmol) in dioxane/THF (6 mL, 1:1) using (1-aminoisoquinolin-5-yl)boronic acid (18a) (149 mg, 0.792 mmol). K₃PO₄ (2 M aqueous solution, 1.056 mL, 2.113 mmol), PCy₃ (29.6 mg, 0.106 mmol), PdCl₂(dppf)-CH₂Cl₂ adduct (43.1 mg, 0.053 mmol) and Pd₂(dba)₃ (48.4 mg, 0.053 mmol) to afford after workup and purification using method-Y, ethyl 2-(2-((5-(1-aminoisoquinolin-5-yl)-1-(tetrahydrofuran-3-yl)-1H-indazol-3-yl)methoxy)-6-methylphenyl)acetate (247b) (120 mg, 42.3% yield) as a clear oil; ¹H NMR (300 MHz, DMSO-d₆) δ 9.29 (s, 1H), 7.95 (d, J=1.8 Hz, 1H), 7.75 (d, J=9.0 Hz, 1H), 7.55 (dd, J=9.0, 1.8 Hz, 1H), 7.48-7.42 (m, 2H), 7.40-7.31 (m, 4H), 7.21-7.04 (m, 2H), 6.82 (d, J=7.3 Hz, 1H), 5.58-5.42 (m, 1H), 5.36 (s, 2H), 4.14-4.01 (m, 2H), 4.01-3.82 (m, 4H), 3.59 (s, 2H), 2.47-2.38 (m, 1H), 2.38-2.22 (m, 1H), 2.20 (s, 3H), 1.00 (t, J=7.1 Hz, 3H); MS (ES+): 537.30 (M+1).

### Step-3: Preparation of 2-(2-((5-(1-aminoisoquinolin-5-yl)-1-(tetrahydrofuran-3-yl)-1H-indazol-3-yl)methoxy)-6-methylphenyl)acetic acid (247c)

Compound 247c was prepared according to the procedure reported in step-2 of scheme-1 from ethyl 2-(2-((5-(1-aminoisoquinolin-5-yl)-1-(tetrahydrofuran-3-yl)-1H-indazol-3-yl)methoxy)-6-methylphenyl)acetate (247b) (120 mg, 0.224 mmol) in MeOH/THF (6 mL, 1:1) using a solution of LiOH·H₂O (56.3 mg, 1.342 mmol) in water (2 mL) to afford after workup and purification using method-G, 2-(2-((5-(1-aminoisoquinolin-5-yl)-1-(tetrahydrofuran-3-yl)-1H-indazol-3-yl)methoxy)-6-methylphenyl)acetic acid (247c) (65 mg, 57.2% yield) HCl salt as a white solid; ¹H NMR (300 MHz, DMSO-d₆) δ 13.34 (s, 1H, D₂O exchangeable), 12.01 (s, 1H, D₂O exchangeable), 9.18 (s, 2H, D₂O exchangeable), 8.63 (d, J=8.3 Hz, 1H), 7.97-7.89 (m, 2H), 7.89-7.80 (m, 2H), 7.63 (d, J=7.2 Hz, 1H), 7.50 (dd, J=8.7, 1.6 Hz, 1H), 7.17-7.05 (m, 2H), 6.97 (d, J=7.2 Hz, 1H), 6.83-6.75 (m, 1H), 5.67-5.51 (m, 1H), 5.42 (s, 2H), 4.23-4.06 (m, 2H), 4.06-3.84 (m, 2H), 3.54 (s, 2H), 2.49-2.29 (m, 2H), 2.17 (s, 3H); MS (ES+): 509.20 (M+1); (ES−): 507.15 (M−1); Analysis calculated for C₃₀H₂₈N₄O₄·HCl·1.5H2O: C, 62.99; H, 5.64; Cl, 6.20; N, 9.79; Found: C, 62.88; H, 5.52; Cl, 6.26; N, 9.77.

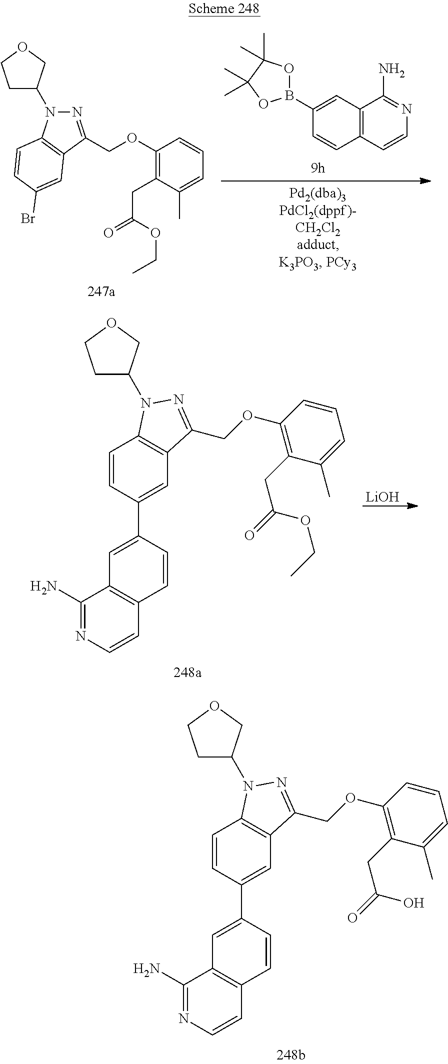

### Preparation of 2-(2-((5-(1-aminoisoquinolin-7-yl)-1-(tetrahydrofuran-3-yl)-1H-indazol-3-yl)methoxy)-6-methylphenyl)acetic acid (248b)

### Step-1: Preparation of ethyl 2-(2-((5-(1-aminoisoquinolin-7-yl)-1-(tetrahydrofuran-3-yl)-1H-indazol-3-yl)methoxy)-6-methylphenyl)acetate (248a)

Compound 248a was prepared according to the procedure reported in step-5 of scheme-1 from ethyl 2-(2-((5-bromo-1-(tetrahydrofuran-3-yl)-1H-indazol-3-yl)methoxy)-6-methylphenyl)acetate (247a) (250 mg, 0.528 mmol) in dioxane/THF (6 mL, 1:1) using 7-(4,4,5,5-tetramethyl-1,3,2-dioxaborolan-2-yl)isoquinolin-1-amine (9h) (214 mg, 0.792 mmol), K₃PO₄ (2 M aqueous solution, 1.056 mL, 2.113 mmol), PCy₃ (29.6 mg, 0.106 mmol), PdCl₂(dppf)-CH₂Cl₂ adduct (43.5 mg, 0.053 mmol) and Pd₂(dba)₃ (48.4 mg, 0.053 mmol) to afford after workup and purification using method-Y, ethyl 2-(2-((5-(1-aminoisoquinolin-7-yl)-1-(tetrahydrofuran-3-yl)-1H-indazol-3-yl)methoxy)-6-methylphenyl)acetate (248a) (120 mg, 42.3% yield) as a clear oil; MS (ES+): 537.30 (M+1).

### Step-2: Preparation of 2-(2-((5-(1-aminoisoquinolin-7-yl)-1-(tetrahydrofuran-3-yl)-1H-indazol-3-yl)methoxy)-6-methylphenyl)acetic acid (248b)

Compound 248b was prepared according to the procedure reported in step-2 of scheme-1 from ethyl 2-(2-((5-(1-aminoisoquinolin-7-yl)-1-(tetrahydrofuran-3-yl)-1H-indazol-3-yl)methoxy)-6-methylphenyl)acetate (248a) (120 mg, 0.224 mmol) in MeOH/THF (6 mL, 1:1) using a solution of LiOH·H₂O (56.3 mg, 1.342 mmol) in water (2 mL) to afford after workup and purification using method-G, 2-(2-((5-(1-aminoisoquinolin-7-yl)-1-(tetrahydrofuran-3-yl)-1H-indazol-3-yl)methoxy)-6-methylphenyl)acetic acid (248b) (65 mg, 57.2% yield) HCl salt as a white solid; ¹H NMR (300 MHz, DMSO-d₆) δ 13.27 (s, 1H, D₂O exchangeable), 12.21 (s, 1H, D₂O exchangeable), 9.17 (s, 2H, D₂O) exchangeable), 8.96 (d, J=1.8 Hz, 1H), 8.39 (dd, J=8.5, 1.6 Hz, 1H), 8.34-8.26 (m, 1H), 8.11-7.99 (m, 2H), 7.94 (d, J=8.9 Hz, 1H), 7.69 (d, J=6.9 Hz, 1H), 7.27 (d, J=6.9 Hz, 1H), 7.22-7.07 (m, 2H), 6.87-6.74 (m, 1H), 5.68-5.52 (m, 1H), 5.46 (s, 2H), 4.24-4.03 (m, 2H), 4.03-3.84 (m, 2H), 3.58 (s, 2H), 2.49-2.29 (m, 2H), 2.19 (s, 3H); MS (ES+): 509.20 (M+1); (ES−): 507.10 (M−1).

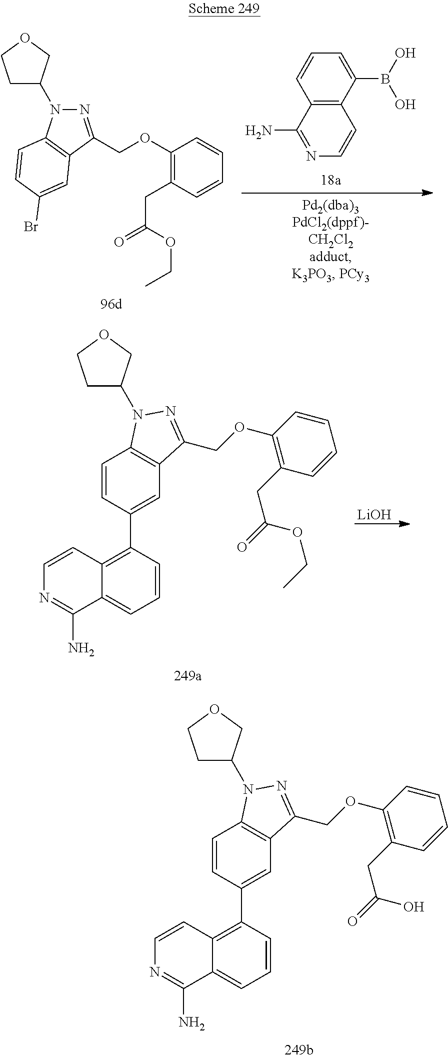 [SMILES: CCOC(=O)Cc1ccccc1OCc1nn(C2CCOC2)c2ccc(Br)cc12 ; Nc1nccc2c(B(O)O)cccc12 ; CCOC(=O)Cc1ccccc1OCc1nn(C2CCOC2)c2ccc(-c3cccc4c(N)nccc34)cc12 ; "LiOH" ; Nc1nccc2c(-c3ccc4c(c3)c(COc3ccccc3CC(=O)O)nn4C3CCOC3)cccc12]

### Preparation of 2-(2-((5-(1-aminoisoquinolin-5-yl)-1-(tetrahydrofuran-3-yl)-1H-indazol-3-yl)methoxy)phenyl)acetic acid (249b)

### Step-1: Preparation of ethyl 2-(2-((5-(1-aminoisoquinolin-5-yl)-1-(tetrahydrofuran-3-yl)-1H-indazol-3-yl)methoxy)phenyl)acetate (249a)

Compound 249a was prepared according to the procedure reported in step-5 of scheme-1 from ethyl 2-(2-((5-bromo-1-(tetrahydrofuran-3-yl)-1H-indazol-3-yl)methoxy)phenyl)acetate (96d) (500 mg, 1.089 mmol) in dioxane/THF (6 mL, 1:1) using (1-aminoisoquinolin-5-yl)boronic acid (18a) (307 mg, 1.633 mmol), K₃PO₄ (2 M aqueous solution, 2.177 mL, 4.35 mmol), PCy₃ (61.1 mg, 0.218 mmol). PdCl₂(dppf)-CH₂Cl₂ adduct (89 mg, 0.109 mmol) and Pd₂(dba)₃ (100 mg, 0.109 mmol) to afford after workup and purification using method-Y, ethyl 2-(2-((5-(1-aminoisoquinolin-5-yl)-1-(tetrahydrofuran-3-yl)-1H-indazol-3-yl)methoxy)phenyl)acetate (249a) (300 mg, 52.7% yield) as a clear oil; MS (ES+): 523.25 (M+1);

### Step-6: Preparation of 2-(2-((5-(1-aminoisoquinolin-5-yl)-1-(tetrahydrofuran-3-yl)-1H-indazol-3-yl)methoxy)phenyl)acetic acid (249b)

Compound 249b was prepared according to the procedure reported in step-2 of scheme-1 from ethyl 2-(2-((5-(1-aminoisoquinolin-5-yl)-1-(tetrahydrofuran-3-yl)-1H-indazol-3-yl)methoxy)phenyl)acetate (249a) (300 mg, 0.574 mmol) in MeOH/THF (6 mL, 1:1) using a solution of LiOH·H₂O (145 mg, 3.44 mmol) in water (2 mL) to afford after workup and purification using method-G, 2-(2-((5-(1-aminoisoquinolin-5-yl)-1-(tetrahydrofuran-3-yl)-1H-indazol-3-yl)methoxy)phenyl)acetic acid (249b) (112 mg, 39.5% yield) HCl salt as a white solid; ¹H NMR (300 MHz, DMSO-d₆) δ 13.70 (s, 1H, D₂O exchangeable), 9.40 (s, 2H, D₂O exchangeable), 8.71 (d, J=8.3 Hz, 1H), 8.00-7.78 (m, 4H), 7.71-7.59 (m, 1H), 7.55-7.44 (m, 1H), 7.28-7.20 (m, 2H), 7.20-7.13 (m, 1H), 6.96 (d, J=7.3 Hz, 1H), 6.93-6.84 (m, 1H), 5.65-5.52 (m, 1H), 5.42 (s, 2H), 4.21-4.04 (m, 2H), 4.04-3.83 (m, 2H), 3.48 (s, 2H), 2.58-2.50 (m, 1H), 2.48-2.28 (m, 1H); MS (ES+): 495.20 (M+1); (ES−): 493.10 (M−1).

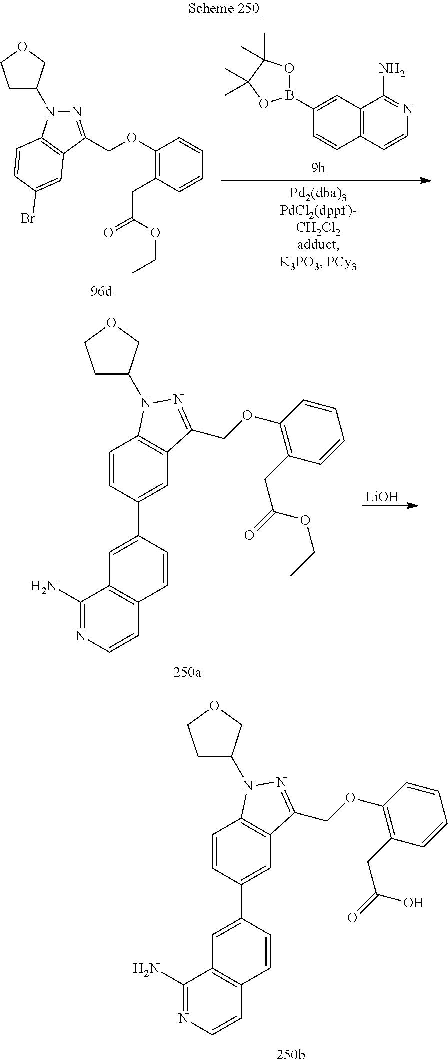

### Preparation of 2-(2-((5-(1-aminoisoquinolin-7-yl)-1-(tetrahydrofuran-3-yl)-1H-indazol-3-yl)methoxy)phenyl)acetic acid (250b)

### Step-1: Preparation of ethyl 2-(2-((5-(1-aminoisoquinolin-7-yl)-1-(tetrahydrofuran-3-yl)˜1H-indazol-3-yl)methoxy)phenyl)acetate (250a)

Compound 250a was prepared according to the procedure reported in step-5 of scheme-1 from ethyl 2-(2-((5-bromo-1-(tetrahydrofuran-3-yl)-1H-indazol-3-yl)methoxy)phenyl)acetate (96d) (500 mg, 1.089 mmol) in dioxane/THF (6 mL, 1:1) using 7-(4,4,5,5-tetramethyl-1,3,2-dioxaborolan-2-yl)isoquinolin-1-amine (9h) (441 mg, 1.633 mmol), K₃PO₄ (2 M aqueous solution, 2.177 mL, 4.35 mmol), PCy₃ (61.1 mg, 0.218 mmol), PdCl₂(dppf)-CH₂Cl₂ adduct (89 mg, 0.109 mmol) and Pd₂(dba)₃ (100 mg, 0.109 mmol) to afford after workup and purification using method-Y, ethyl 2-(2-((5-(1-aminoisoquinolin-7-yl)-1-(tetrahydrofuran-3-yl)-1H-indazol-3-yl)methoxy)phenyl)acetate (250a) (300 mg, 52.7% yield) as a clear oil; MS (ES+): 523.20 (M+1).

### Step-2: Preparation of 2-(2-((5-(1-aminoisoquinolin-7-yl)-1-(tetrahydrofuran-3-yl)-1H-indazol-3-yl)methoxy)phenyl)acetic acid (250b)

Compound 250b was prepared according to the procedure reported in step-2 of scheme-1 from ethyl 2-(2-((5-(1-aminoisoquinolin-7-yl)-1-(tetrahydrofuran-3-yl)-1H-indazol-3-yl)methoxy)phenyl)acetate (250a) (300 mg, 0.574 mmol) in MeOH/THF (6 mL, 1:1) using a solution of LiOH·H₂O (145 mg, 3.44 mmol) in water (2 mL) to afford after workup and purification using method-G, 2-(2-((5-(1-aminoisoquinolin-7-yl)-1-(tetrahydrofuran-3-yl)-˜527-1H-indazol-3-yl)methoxy)phenyl)acetic acid (250b) (190 mg, 66.9% yield) HCl salt as a white solid; ¹H NMR (300 MHz, DMSO-d₆) δ 13.27 (s, 1H, D₂O exchangeable), 12.31 (s, 1H, D₂O exchangeable), 9.26 (s, 2H, D₂O exchangeable), 9.04-8.94 (m, 1H), 8.41 (dd, J=8.5, 1.6 Hz, 1H), 8.35-8.27 (m, 1H), 8.09-7.98 (m, 2H), 7.93 (d, J=8.9 Hz, 1H), 7.69 (d, J=6.9 Hz, 1H), 7.33-7.22 (m, 3H), 7.22-7.16 (m, 1H), 6.97-6.85 (m, 1H), 5.65-5.51 (m, 1H), 5.48 (s, 2H), 4.21-4.03 (m, 2H), 4.03-3.83 (m, 2H), 3.53 (s, 2H), 2.49-2.42 (m, 1H), 2.42-2.29 (m, 1H); MS (ES+): 495.20 (M+1); (ES−): 493.10 (M−1).

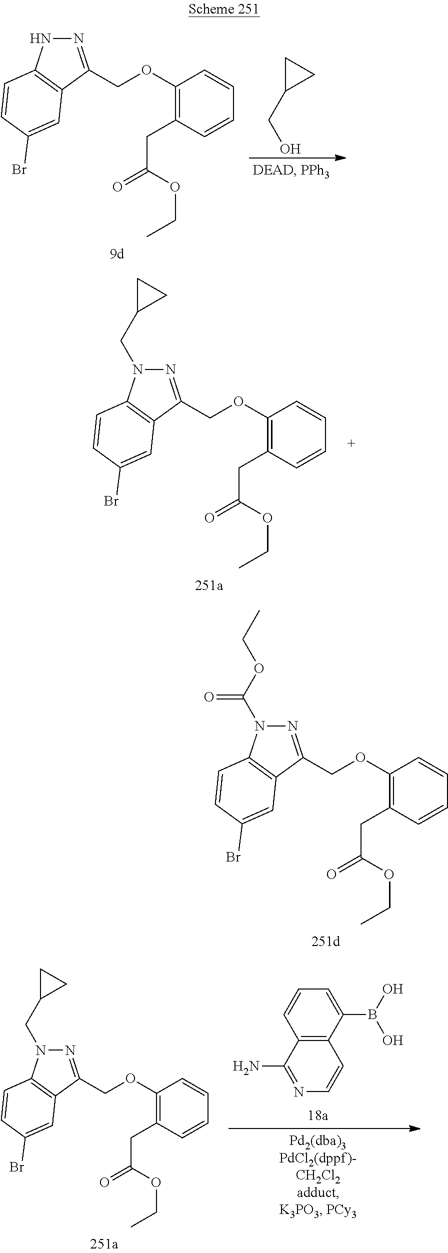

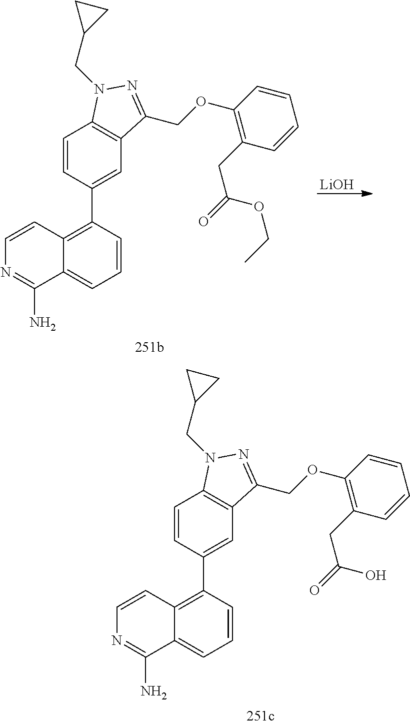 [SMILES: CCOC(=O)Cc1ccccc1OCc1nn(CC2CC2)c2ccc(-c3cccc4c(N)nccc34)cc12 ; "LiOH" ; Nc1nccc2c(-c3ccc4c(c3)c(COc3ccccc3CC(=O)O)nn4CC3CC3)cccc12]

### Preparation of 2-(2-((5-(1-aminoisoquinolin-5-yl)-1-(cyclopropylmethyl)-1H-indazol-3-yl)methoxy)phenyl)acetic acid (251c)

### Step-1: Preparation of ethyl 2-(2-((5-bromo-1-(cyclopropylmethyl)-1H-indazol-3-yl)methoxy)phenyl)acetate (251a) and ethyl 5-bromo-3-((2-(2-ethoxy-2-oxoethyl)phenoxy)methyl)-1H-indazole-1-carboxylate (251d)

Compounds 251a and 251d were prepared according to the procedure reported in step-1 of scheme-8 from ethyl 2-(2-((5-bromo-1H-indazol-3-yl)methoxy)phenyl)acetate (9d) (500 mg, 1.285 mmol) in DCM (10 mL) using cyclopropylmethanol (185 mg, 2.57 mmol; CAS #2516-33-8), PPh₃ (674 mg, 2.57 mmol) using a solution of DEAD (447 mg, 2.57 mmol) in DCM (10 mL) to afford after workup and purification using method-L, ethyl 2-(2-((5-bromo-1-(cyclopropylmethyl)-1H-indazol-3-yl)methoxy)phenyl)acetate (251a) (80 mg, 14.05% yield) as a yellow oil; ¹H NMR (300 MHz, DMSO-d₆) δ 7.96-7.92 (m, 1H), 7.78-7.70 (m, 1H), 7.52 (dd, J=8.9, 1.9 Hz, 1H), 7.31-7.17 (m, 3H), 6.97-6.87 (m, 1H), 5.38 (s, 2H), 4.31 (d, J=7.0 Hz, 2H), 3.92 (q, J=7.1 Hz, 2H), 3.53 (s, 2H), 1.34-1.17 (m, 1H), 0.95 (t, J=7.1 Hz, 3H), 0.55-0.33 (m, 4H); MS (ES+): 443.10 & 445.05 (M+1) and ethyl 5-bromo-3-((2-(2-ethoxy-2-oxoethyl)phenoxy)methyl)-1H-indazole-1-carboxylate (251d) (200 mg, 33.8% yield) as a yellow oil.

### Step-2: Preparation of ethyl 2-(2-((5-(1-aminoisoquinolin-5-yl)-1-(cyclopropylmethyl)-1H-indazol-3-yl)methoxy)phenyl)acetate (251b)

Compound 251b was prepared according to the procedure reported in step-5 of scheme-1 from ethyl 2-(2-((5-bromo-1-(cyclopropylmethyl)-1H-indazol-3-yl)methoxy)phenyl)acetate (251a) (80 mg, 0.180 mmol) in dioxane/THF (6 mL, 1:1) using (1-aminoisoquinolin-5-yl)boronic acid (18a) (50.9 mg, 0.271 mmol), K₃PO₄ (2 M aqueous solution, 0.361 mL, 0.722 mmol), PCy₃ (10.12 mg, 0.036 mmol), PdCl₂(dppf)-CH₂Cl₂ adduct (14.74 mg, 0.018 mmol) and Pd₂(dba)₃ (16.52 mg, 0.018 mmol) to afford after workup and purification using method-Y, ethyl 2-(2-((5-(1-aminoisoquinolin-5-yl)-1-(cyclopropylmethyl)-1H-indazol-3-yl)methoxy)phenyl)acetate (251b) (80 mg, 88% yield) as a clear oil; MS (ES+): 507.20 (M+1).

### Step-3: Preparation of 2-(2-((5-(1-aminoisoquinolin-5-yl)-1-(cyclopropylmethyl)-1H-indazol-3-yl)methoxy)phenyl)acetic acid (251c)

Compound 251c was prepared according to the procedure reported in step-2 of scheme-1 from ethyl 2-(2-((5-(1-aminoisoquinolin-5-yl)-1-(cyclopropylmethyl)-1H-indazol-3-yl)methoxy)phenyl)acetate (251b) (80 mg, 0.158 mmol) in MeOH/THF (6 mL, 1:1) using a solution of LiOH·H₂O (39.8 mg, 0.948 mmol) in water (2 mL) to afford after workup and purification using method-G, 2-(2-((5-(1-aminoisoquinolin-5-yl)-1-(cyclopropylmethyl)-1H-indazol-3-yl)methoxy)phenyl)acetic acid (251c) (20 mg, 26.5% yield) HCl salt as a white solid; ¹H NMR (300 MHz, DMSO-d₆) δ 13.17 (s, 1H, D₂O) exchangeable), 12.02 (s, 1H, D₂O exchangeable), 9.08 (s, 2H, D₂O exchangeable), 8.60 (d, J=8.3 Hz, 1H), 8.02-7.80 (m, 4H), 7.62 (d, J=7.2 Hz, 1H), 7.47 (dd, J=8.6, 1.7 Hz, 1H), 7.30-7.20 (m, 2H), 7.20-7.13 (m, 1H), 7.00 (d, J=7.2 Hz, 1H), 6.94-6.85 (m, 1H), 5.45 (s, 2H), 4.39 (d, J=7.0 Hz, 2H), 3.48 (s, 2H), 1.43-1.24 (m, 1H), 0.59-0.41 (m, 4H); MS (ES+): 479.20 (M+1); (ES−): 477.10 (M−1).

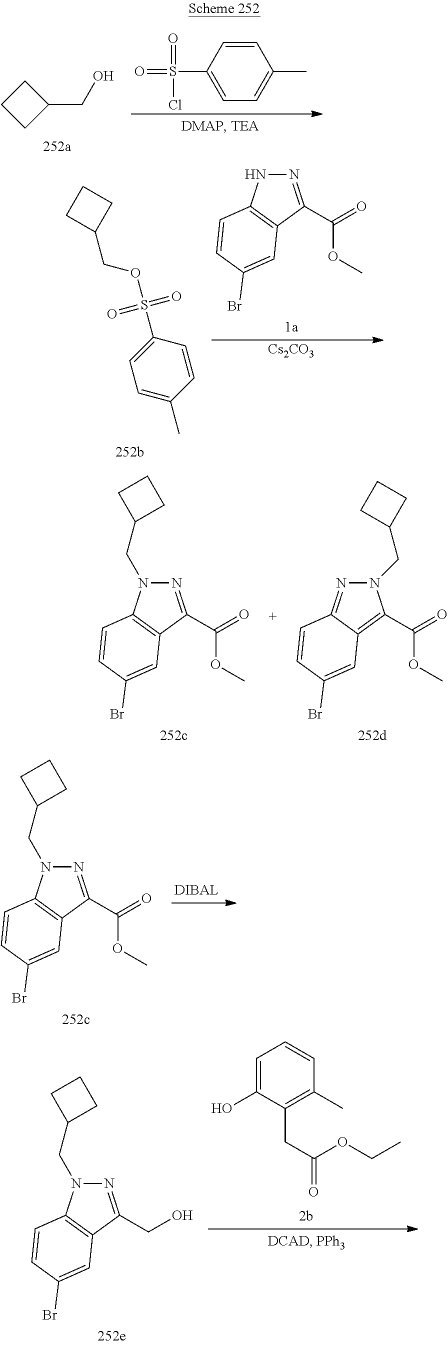

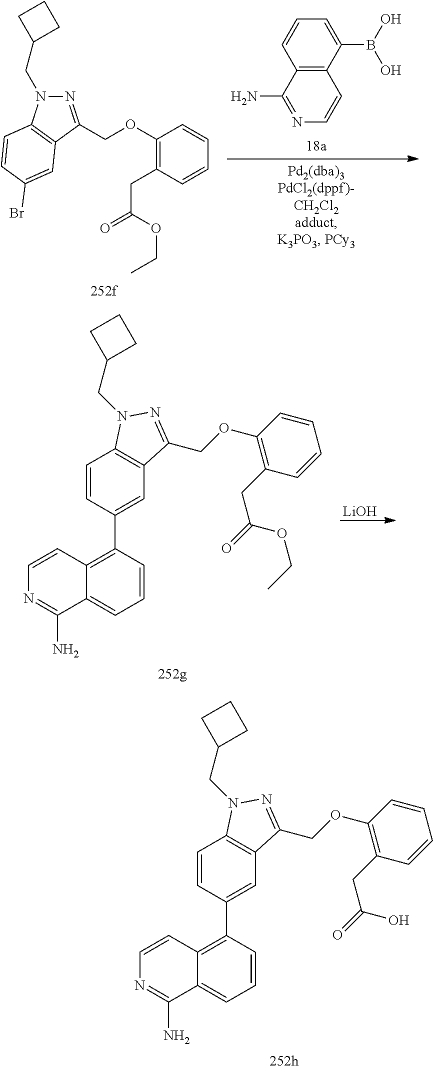

### Preparation of 2-(2-((5-(1-aminoisoquinolin-5-yl)-1-(cyclobutylmethyl)-1H-indazol-3-yl)methoxy)phenyl)acetic acid (252h)

### Step-1: Preparation of cyclobutylmethyl 4-methylbenzenesulfonate (252b)

Compound 252b was prepared according to the procedure reported in step-1 of scheme-86 from cyclobutylmethanol (252a) (3 g, 34.8 mmol; CAS #4415-82-1) in anhydrous DCM (80 mL) using DMAP (0.213 g, 1.742 mmol), TEA (7.28 mL, 52.2 mmol) and p-toluenesulfonylchloride (13.28 g, 69.7 mmol) to afford after workup and purification using method-O, cyclobutylmethyl 4-methylbenzenesulfonate (252b) (7.3 g, 87% yield) as a clear oil; ¹H NMR (300 MHz, DMSO-d₆) δ 7.91-7.67 (m, 2H), 7.57-7.33 (m, 2H), 3.97 (d, J=6.7 Hz, 2H), 2.58-2.51 (m, 1H), 2.41 (s, 3H), 2.00-1.53 (m, 6H); MS (ES+): 263.00 (M+Na).

### Step-2: Preparation of methyl 5-bromo-1-(cyclobutylmethyl)-1H-indazole-3-carboxylate (252c) and methyl 5-bromo-2-(cyclobutylmethyl)-2H-indazole-3-carboxy late (252d)

Compounds 252c and 252d were prepared according to the procedure reported in step-2 of scheme-86 from methyl 5-bromo-1H-indazole-3-carboxylate (1a) (3.87 g, 15.19 mmol) in DMF (40 mL) using Cs₂CO₃ (9.90 g, 30.4 mmol) and cyclobutylmethyl 4-methylbenzenesulfonate (252b) (7.3 g, 30.4 mmol) to afford after workup and purification using method-A, methyl 5-bromo-1-(cyclobutylmethyl)-1H-indazole-3-carboxylate (252c) (2.3 g, 46.9% yield) as a yellow solid; ¹H NMR (300 MHz, DMSO-d₆) δ 8.19 (dd, J=1.9, 0.7 Hz, 1H), 7.90 (dd, J=9.0, 0.7 Hz, 1H), 7.62 (dd, J=9.0, 1.9 Hz, 1H), 4.55 (d, J=7.3 Hz, 2H), 3.92 (s, 3H), 2.96-2.74 (m, 1H), 1.98-1.69 (m, 6H); MS (ES+): 323.00 & 325.00 (M+1); 345.00 & 347.00 (M+Na) and methyl 5-bromo-2-(cyclobutylmethyl)-2H-indazole-3-carboxylate (252d) (1.0 g, 20.37% yield) as a yellow solid; ¹H NMR (300 MHz, DMSO-d₆) δ 8.14 (dd, J=1.9, 0.8 Hz, 1H), 7.78 (dd, J=9.0, 0.8 Hz, 1H), 7.47 (dd, J=9.1, 1.9 Hz, 1H), 4.86 (d, J=7.2 Hz, 2H), 3.98 (s, 3H), 2.98-2.79 (m, 1H), 2.08-1.70 (m, 6H); MS (ES+): 323.00 & 325.00 (M+1); 345.00 & 347.00 (M+Na).

### Step-3: Preparation of (5-bromo-1-(cyclobutylmethyl)-1H-indazol-3-yl)methanol (252e)

Compound 252e was prepared according to the procedure reported in step-1 of scheme-2 from methyl 5-bromo-1-(cyclobutylmethyl)-1H-indazole-3-carboxylate (252c) (2.2 g, 6.81 mmol) in DCM (40 mL) using DIBAL (1M solution in DCM, 17.02 mL, 17.02 mmol) to afford after workup and purification using method-F, (5-bromo-1-(cyclobutylmethyl)-1H-indazol-3-yl)methanol (252e) (1.7 g, 85% yield) as a light yellow oil; ¹H NMR (300 MHz, DMSO-d₆) δ 8.04 (d, J=1.9 Hz, 1H), 7.67 (d, J=8.9 Hz, 1H), 7.47 (dd. J=8.9, 1.9 Hz, 1H), 5.32 (s, 1H), 4.74 (s, 2H), 4.36 (d, J=7.2 Hz, 2H), 2.87-2.68 (m, 1H), 1.94-1.71 (m, 6H); MS (ES+): 295.00 & 297.00 (M+1).

### Step-4: Preparation of ethyl 2-(2-((5-bromo-1-(cyclobutylmethyl)-1H-indazol-3-yl)methoxy)phenyl)acetate (252f)

Compound 252f was prepared according to the procedure reported in step-2 of scheme-2 from (5-bromo-1-(cyclobutylmethyl)-1H-indazol-3-yl)methanol (252e) (1.7 g, 5.76 mmol) in DCM (20 mL) using ethyl 2-(2-hydroxyphenyl)acetate (2b) (1.09 g, 6.05 mmol), PPh₃ (1.813 g, 6.91 mmol) and a solution of DCAD (2.54 g, 6.91 mmol) in DCM (20 mL) to afford after workup and purification using method-N, ethyl 2-(2-((5-bromo-1-(cyclobutylmethyl)-1H-indazol-3-yl)methoxy)phenyl)acetate (252f) (1.5 g, 56.9% yield) as a white oil; ¹H NMR (300 MHz, DMSO-d₆) δ 7.94 (dd, J=1.8, 0.7 Hz, 1H), 7.73 (dd, J=8.9, 0.7 Hz, 1H), 7.52 (dd. J=8.9, 1.8 Hz, 1H), 7.27-7.17 (m, 2H), 6.95-6.86 (m, 1H), 6.81-6.69 (m, 1H), 5.37 (s, 2H), 4.43 (d, J=7.1 Hz, 2H), 3.91 (q, J=7.1 Hz, 2H), 3.53 (s, 3H), 2.81 (p. J=7.3 Hz, 1H), 1.98-1.70 (m, 6H), 0.94 (t, J=7.1 Hz, 3H); MS (ES+): 457.10 & 459.10 (M+1); 479.10 & 481.10 (M+Na).

### Step-5: Preparation of ethyl 2-(2-((5-(1-aminoisoquinolin-5-yl)-1-(cyclobutylmethyl)-1H-indazol-3-yl)methoxy)phenyl)acetate (252g)

Compound 252g was prepared according to the procedure reported in step-5 of scheme-1 from ethyl 2-(2-((5-bromo-1-(cyclobutylmethyl)-1H-indazol-3-yl)methoxy)phenyl)acetate (252f) (200 mg, 0.437 mmol) in dioxane/THF (4 mL, 1:1) was added (1-aminoisoquinolin-5-yl)boronic acid (18a) (123 mg, 0.656 mmol), K₃PO₄ (2 M aqueous solution, 0.875 mL, 1.749 mmol), PCy₃ (24.53 mg, 0.087 mmol), PdCl₂(dppf)-CH₂Cl₂ adduct (35.7 mg, 0.044 mmol) and Pd₂(dba)₃ (40.0 mg, 0.044 mmol) to afford after workup and purification using method-Y, ethyl 2-(2-((5-(1-aminoisoquinolin-5-yl)-1-(cyclobutylmethyl)-1H-indazol-3-yl)methoxy)phenyl)acetate (252g) (170 mg, 74.7% yield) as a clear oil; ¹H NMR (300 MHZ, DMSO-d₆) δ 8.22 (d, J=8.2 Hz, 1H), 7.86-7.72 (m, 3H), 7.63-7.57 (m, 1H), 7.54 (d, J=7.9 Hz, 1H), 7.52-7.45 (m, 1H), 7.27-7.21 (m, 2H), 7.15 (d, J=7.2 Hz, 1H), 6.94-6.85 (m, 3H), 6.82 (d, J=6.1 Hz, 1H), 5.42 (s, 2H), 4.49 (d, J=7.0 Hz, 2H), 3.55 (q, J=7.1 Hz, 2H), 3.50 (s, 2H), 2.88 (p, J=7.1 Hz, 1H), 2.02-1.82 (m, 6H), 0.71 (t, J=7.1 Hz, 3H); MS (ES+): 521.25 (M+1).

### Step-6: Preparation of 2-(2-((5-(1-aminoisoquinolin-5-yl)-1-(cyclobutylmethyl)-1H-indazol-3-yl)methoxy)phenyl)acetic acid (252h)

Compound 252h was prepared according to the procedure reported in step-2 of scheme-1 from ethyl 2-(2-((5-(1-aminoisoquinolin-5-yl)-1-(cyclobutylmethyl)-1H-indazol-3-yl)methoxy)phenyl)acetate (252g) (170 mg, 0.327 mmol) in MeOH/THF (6 mL, 1:1) using a solution of LiOH·H₂O (82 mg, 1.959 mmol) in water (2 mL) to afford after workup and purification using method-G, 2-(2-((5-(1-aminoisoquinolin-5-yl)-1-(cyclobutylmethyl)-1H-indazol-3-yl)methoxy)phenyl)acetic acid (252h) (60 mg, 37.3% yield) HCl salt as a white solid; ¹H NMR (300 MHz, DMSO-d₆) δ 13.43 (s, 1H, D₂O exchangeable), 12.04 (s, 1H, D₂O exchangeable), 9.22 (s, 2H, D₂O exchangeable), 8.64 (d, J=8.3 Hz, 1H), 8.00-7.92 (m, 1H), 7.91-7.79 (m, 3H), 7.63 (d, J=7.2 Hz, 1H), 7.51-7.42 (m, 1H), 7.27-7.20 (m, 2H), 7.20-7.12 (m, 1H), 6.97 (d, J=7.2 Hz, 1H), 6.94-6.82 (m, 1H), 5.44 (s, 2H), 4.50 (d, J=7.1 Hz, 2H), 3.47 (s, 2H), 2.97-2.78 (m, 1H), 2.04-1.77 (m, 6H); MS (ES+): 493.20 (M+1); (ES−): 491.10 (M−1).

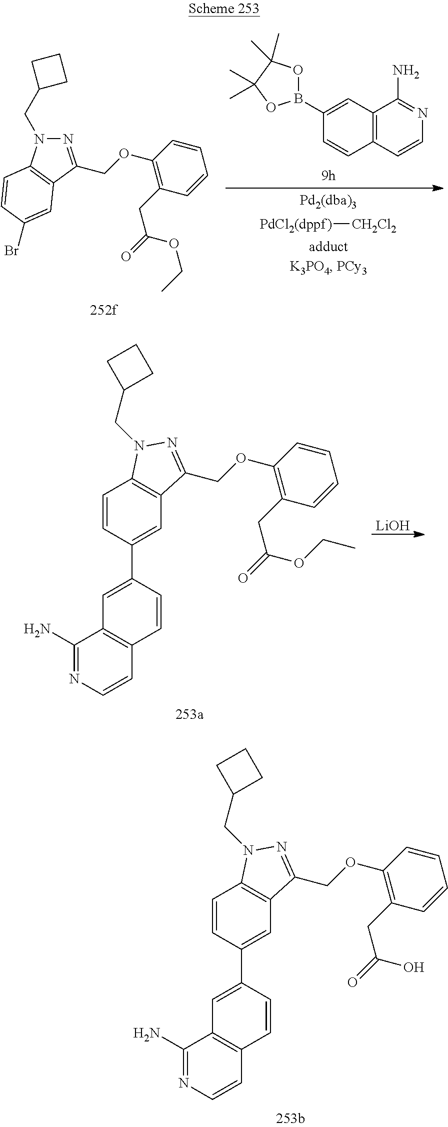

### Preparation of 2-(2-((5-(1-aminoisoquinolin-7-yl)-1-(cyclobutylmethyl)-1H-indazol-3-yl)methoxy)phenyl)acetic acid (253b)

### Step-1: Preparation of ethyl 2-(2-((5-(1-aminoisoquinolin-7-yl)-1-(cyclobutylmethyl)-1H-indazol-3-yl)methoxy)phenyl)acetate (253a)

Compound 253a was prepared according to the procedure reported in step-5 of scheme-1 from ethyl 2-(2-((5-bromo-1-(cyclobutylmethyl)-1H-indazol-3-yl)methoxy)phenyl)acetate (252f) (200 mg, 0.437 mmol) in dioxane/THF (4 mL, 1:1) using 7-(4,4,5,5-tetramethyl-1,3,2-dioxaborolan-2-yl)isoquinolin-1-amine (9h) (177 mg, 0.656 mmol), K₃PO₄ (2 M aqueous solution, 0.875 mL, 1.749 mmol), PCy₃ (24.53 mg, 0.087 mmol), PdCl₂(dppf)-CH₂Cl₂ adduct (35.7 mg, 0.044 mmol) and Pd₂(dba)₃ (40.0 mg, 0.044 mmol) to afford after workup and purification using method-Y, ethyl 2-(2-((5-(1-aminoisoquinolin-7-yl)-1-(cyclobutylmethyl)˜ 1H-indazol-3-yl)methoxy)phenyl)acetate (253a) (170 mg, 74.7% yield) as a clear oil; MS (ES+): 521.30 (M+1); MS (ES−): 519.20 (M−1).

### Step-2: Preparation of 2-(2-((5-(1-aminoisoquinolin-7-yl)-1-(cyclobutylmethyl)-1H-indazol-3-yl)methoxy)phenyl)acetic acid (253b)

Compound 253b was prepared according to the procedure reported in step-2 of scheme-1 from ethyl 2-(2-((5-(1-aminoisoquinolin-7-yl)-1-(cyclobutylmethyl)-1H-indazol-3-yl)methoxy)phenyl)acetate (253a) (170 mg, 0.327 mmol) in MeOH/THF (6 mL, 1:1) using a solution of LiOH·H₂O (82 mg, 1.959 mmol) in water (2 mL) to afford after workup and purification using method-G, 2-(2-((5-(1-aminoisoquinolin-7-yl)-1-(cyclobutylmethyl)-1H-indazol-3-yl)methoxy)phenyl)acetic acid (253b) (60 mg, 37.3% yield) HCl salt as a white solid; ¹H NMR (300 MHz, DMSO-d₆) δ 9.25 (s, 2H, D₂O exchangeable), 8.98 (d, 1H), 8.40 (dd, 1H), 8.31 (d, J=1.6 Hz, 1H), 8.08-7.95 (m, 2H), 7.95-7.85 (m, 1H), 7.70 (d, J=6.9 Hz, 1H), 7.33-7.22 (m, 3H), 7.22-7.15 (m, 1H), 6.97-6.82 (m, 1H), 5.48 (s, 2H), 4.49 (d, J=7.1 Hz, 2H), 3.52 (s, 2H), 2.96-2.74 (m, 1H), 2.01-1.75 (m, 6H); MS (ES+): 493.20 (M+1); (ES−): 491.15 (M−1).

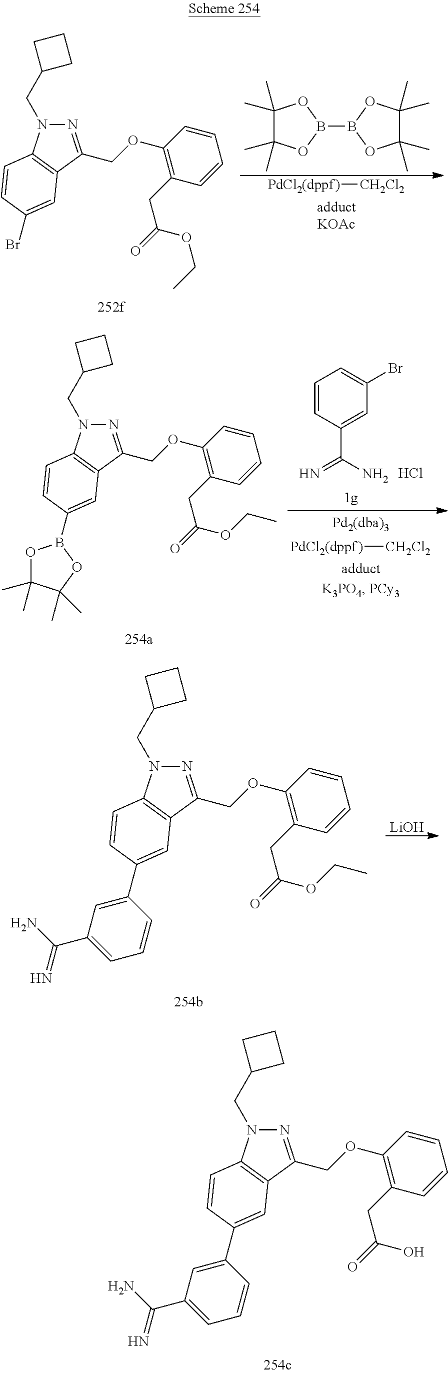

### Preparation of 2-(2-((5-(3-carbamimidoylphenyl)-1-(cyclobutylmethyl)-1H-indazol-3-yl)methoxy)phenyl)acetic acid (254c)

### Step-1: Preparation of ethyl 2-(2-((1-(cyclobutylmethyl)-5-(4,4,5,5-tetramethyl-1,3,2-dioxaborolan-2-yl)-1H-indazol-3-yl)methoxy)phenyl)acetate (254a)

Compound 254a was prepared according to the procedure reported in step-4 of scheme-1 from ethyl 2-(2-((5-bromo-1-(cyclobutylmethyl)-1H-indazol-3-yl)methoxy)phenyl)acetate (252f) (1.1 g, 2.405 mmol) in anhydrous dioxane (20 mL) using BISPIN (1.221 g, 4.81 mmol), KOAc (0.590 g, 6.01 mmol) and PdCl₂(dppf)-CH₂Cl₂ adduct (0.118 g, 0.144 mmol) to afford after workup and purification using method-L, ethyl 2-(2-((1-(cyclobutylmethyl)-5-(4,4,5,5-tetramethyl-1,3,2-dioxaborolan-2-yl)-1H-indazol-3-yl)methoxy)phenyl)acetate (254a) (1.1 g, 91% yield) as a clear oil; ¹H NMR (300 MHz, DMSO-d₆) δ 8.15 (s, 1H), 7.69-7.65 (m, 1H), 7.28-7.26 (m, 1H), 7.19 (d, J=7.3 Hz, 1H), 7.13-7.02 (m, 1H), 6.96-6.87 (m, 1H), 6.80-6.72 (m, 1H), 5.39 (s, 2H), 4.43 (d, J=7.1 Hz, 2H), 3.86 (q, J=7.1 Hz, 2H), 3.50 (s, 2H), 2.94-2.68 (m, 1H), 1.96-1.76 (m, 6H), 1.29 (s, 12H), 0.89 (t, J=7.1 Hz, 3H); MS (ES+): 505.30 (M+1).

### Step-2: Preparation of ethyl 2-(2-((5-(3-carbamimidoylphenyl)-1-(cyclobutylmethyl)-1H-indazol-3-yl)methoxy)phenyl)acetate (254b)

Compound 254b was prepared according to the procedure reported in step-5 of scheme-1 from ethyl 2-(2-((1-(cyclobutylmethyl)-5-(4,4,5,5-tetramethyl-1,3,2-dioxaborolan-2-yl)-1H-indazol-3-yl)methoxy)phenyl)acetate (254a) (500 mg, 0.991 mmol) in dioxane/THF (8 mL, 1:1) using 3-bromobenzimidamide hydrochloride (1g) (467 mg, 1.982 mmol), K₃PO₄ (2 M aqueous solution, 1.982 mL, 3.96 mmol), PCy₃ (55.6 mg, 0.198 mmol). PdCl₂(dppf)-CH₂Cl₂ adduct (81 mg, 0.099 mmol) and Pd₂(dba)₃ (91 mg, 0.099 mmol) to afford after workup and purification using method-AI, to give ethyl 2-(2-((5-(3-carbamimidoylphenyl)-1-(cyclobutylmethyl)-1H-indazol-3-yl)methoxy)phenyl)acetate (254b) (400 mg, 81% yield) as a clear oil; MS (ES+): 497.30 (M+1); 519.20 (M+Na).

### Step-3: Preparation of 2-(2-((5-(3-carbamimidoylphenyl)-1-(cyclobutylmethyl)-1H-indazol-3-yl)methoxy)phenyl)acetic acid (254c)

Compound 254c was prepared according to the procedure reported in step-2 of scheme-1 from ethyl 2-(2-((5-(3-carbamimidoylphenyl)-1-(cyclobutylmethyl)-1H-indazol-3-yl)methoxy)phenyl)acetate (254b) (400 mg, 0.805 mmol) in MeOH/THF (6 mL, 1:1) using a solution of LiOH·H₂O (203 mg, 4.83 mmol) in water (2 mL) to afford after workup and purification using method-G, 2-(2-((5-(3-carbamimidoylphenyl)-1-(cyclobutylmethyl)-1H-indazol-3-yl)methoxy)phenyl)acetic acid (254c) (60 mg, 15.90% yield) HCl salt as a white solid; ¹H NMR (300 MHz, DMSO-d₆) δ 12.10 (s, 1H, D₂O exchangeable), 9.51 (s, 2H, D₂O exchangeable), 9.30 (s, 2H, D₂O exchangeable), 8.23-8.14 (m, 2H), 8.13-8.04 (m, 1H), 7.94-7.83 (m, 2H), 7.83-7.75 (m, 1H), 7.70 (t, J=7.8 Hz, 1H), 7.32-7.22 (m, 2H), 7.22-7.13 (m, 1H), 6.96-6.84 (m, 1H), 5.46 (s, 2H), 4.48 (d, J=7.1 Hz, 2H), 3.52 (s, 2H), 2.93-2.78 (m, 1H), 2.03-1.70 (m, 6H); MS (ES+): 469.20 (M+1); (ES−): 467.20 (M−1).

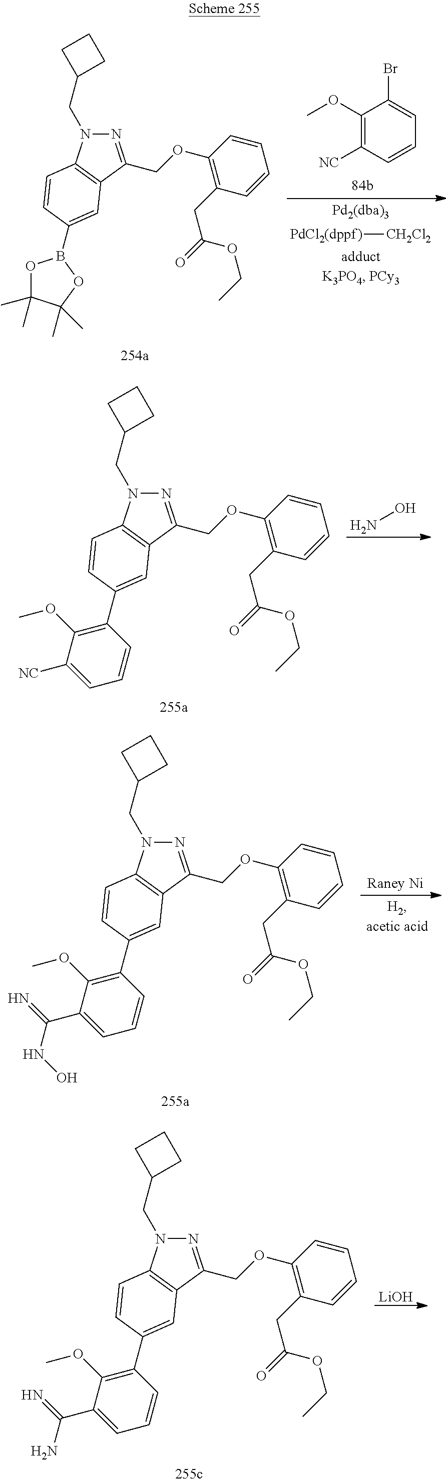

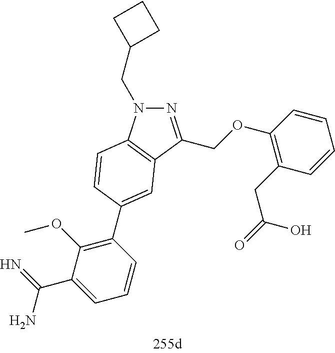 [SMILES: COc1c(C(=N)N)cccc1-c1ccc2c(c1)c(COc1ccccc1CC(=O)O)nn2CC1CCC1]

### Preparation of 2-(2-((5-(3-carbamimidoyl-2-methoxyphenyl)-1-(cyclobutylmethyl)-1H-indazol-3-yl)methoxy)phenyl)acetic acid (255d)

### Step-1: Preparation of ethyl 2-(2-((5-(3-cyano-2-methoxyphenyl)-1-(cyclobutylmethyl)-1H-indazol-3-yl)methoxy)phenyl)acetate (255a)

Compound 255a was prepared according to the procedure reported in step-5 of scheme-1 from ethyl 2-(2-((1-(cyclobutylmethyl)-5-(4,4,5,5-tetramethyl-1,3,2-dioxaborolan-2-yl)-1H-indazol-3-yl)methoxy)phenyl)acetate (254a) (600 mg, 1.189 mmol) in dioxane/THF (8 mL, 1:1) using 3-bromo-2-methoxybenzonitrile (84b) (378 mg, 1.784 mmol), K₃PO₄ (2 M aqueous solution, 2.379 mL, 4.76 mmol), PCy₃ (66.7 mg, 0.238 mmol), PdCl₂(dppf)-CH₂Cl₂ adduct (97 mg, 0.119 mmol) and Pd₂(dba)₃ (109 mg, 0.119 mmol) to afford after workup and purification using method-P, ethyl 2-(2-((5-(3-cyano-2-methoxyphenyl)-1-(cyclobutylmethyl)-1H-indazol-3-yl)methoxy)phenyl)acetate (255a) (440 mg, 72.6% yield) as a clear oil; MS (ES+): 510.15 (M+1); 532.15 (M+Na).

### Step-2: Preparation of ethyl 2-(2-((1-(cyclobutylmethyl)-5-(3-(N-hydroxycarbamimidoyl)-2-methoxyphenyl)-1H-indazol-3-yl)methoxy)phenyl)acetate (255b)

Compound 255b was prepared according to the procedure reported in step-5 of scheme-23 from ethyl 2-(2-((5-(3-cyano-2-methoxyphenyl)-1-(cyclobutylmethyl)-1H-indazol-3-yl)methoxy)phenyl)acetate (255a) (440 mg, 0.863 mmol) in EtOH (10 mL) using hydroxylamine (0.570 mL, 8.63 mmol) to afford after workup and purification using method-E, ethyl 2-(2-((1-(cyclobutylmethyl)-5-(3-(N-hydroxycarbamimidoyl)-2-methoxyphenyl)-1H-indazol-3-yl)methoxy)phenyl)acetate (255b) (350 mg, 74.7% yield) as a white solid; MS (ES+): 543.30 (M+1).

### Step-3: Preparation of ethyl 2-(2-((5-(3-carbamimidoyl-2-methoxyphenyl)-1-(cyclobutylmethyl)-1H-indazol-3-yl)methoxy)phenyl)acetate (255c)

Compound 255c was prepared according to the procedure reported in step-1 of scheme-24 from ethyl 2-(2-((1-(cyclobutylmethyl)-5-(3-(N-hydroxycarbamimidoyl)-2-methoxyphenyl)-1H-indazol-3-yl)methoxy)phenyl)acetate (255b) (350 mg, 0.645 mmol) in EtOH (10 mL) using AcOH (0.037 mL, 0.645 mmol) Raney Nickel (0.645 mmol) and hydrogen (1 atm) to afford after workup and purification using method-AI, ethyl 2-(2-((5-(3-carbamimidoyl-2-methoxyphenyl)-1-(cyclobutylmethyl)-1H-indazol-3-yl)methoxy)phenyl)acetate (255c) (300 mg, 88% yield) as a colorless oil; MS (ES+): 527.30 (M+1).

### Step-4: Preparation of 2-(2-((5-(3-carbamimidoyl-2-methoxyphenyl)-1-(cyclobutylmethyl)-1H-indazol-3-yl)methoxy)phenyl)acetic acid (255d)

Compound 255d was prepared according to the procedure reported step-2 of scheme-1 from ethyl 2-(2-((5-(3-carbamimidoyl-2-methoxyphenyl)-1-(cyclobutylmethyl)-1H-indazol-3-yl)methoxy)phenyl)acetate (255c) (300 mg, 0.570 mmol) in MeOH/THF (6 mL, 1:1) using a solution of LiOH·H₂O (143 mg, 3.42 mmol) in water (2 mL) to afford after workup and purification using method-G, 2-(2-((5-(3-carbamimidoyl-2-methoxyphenyl)-1-(cyclobutylmethyl)-1H-indazol-3-yl)methoxy)phenyl)acetic acid (255d) (180 mg, 63.4% yield) HCl salt as a white solid; ¹H NMR (300 MHz, DMSO-d₆) δ 12.10 (s, 1H, D₂O exchangeable), 9.40 (s, 2H, D₂O exchangeable), 9.36 (s, 2H, D₂O exchangeable), 8.02-7.90 (m, 1H), 7.91-7.79 (m, 1H), 7.70 (dd, J=7.7, 1.8 Hz, 1H), 7.62 (dd, J=8.8, 1.6 Hz, 1H), 7.53 (dd, J=7.7, 1.7 Hz, 1H), 7.40 (t, J=7.7 Hz, 1H), 7.29-7.21 (m, 2H), 7.21-7.13 (m, 1H), 6.97-6.84 (m, 1H), 5.44 (s, 2H), 4.47 (d, J=7.1 Hz, 2H), 3.50 (s, 2H), 3.37 (s, 3H), 2.96-2.72 (m, 1H), 2.03-1.68 (m, 6H); MS (ES+): 499.20 (M+1); (ES−): 497.10 (M−1).

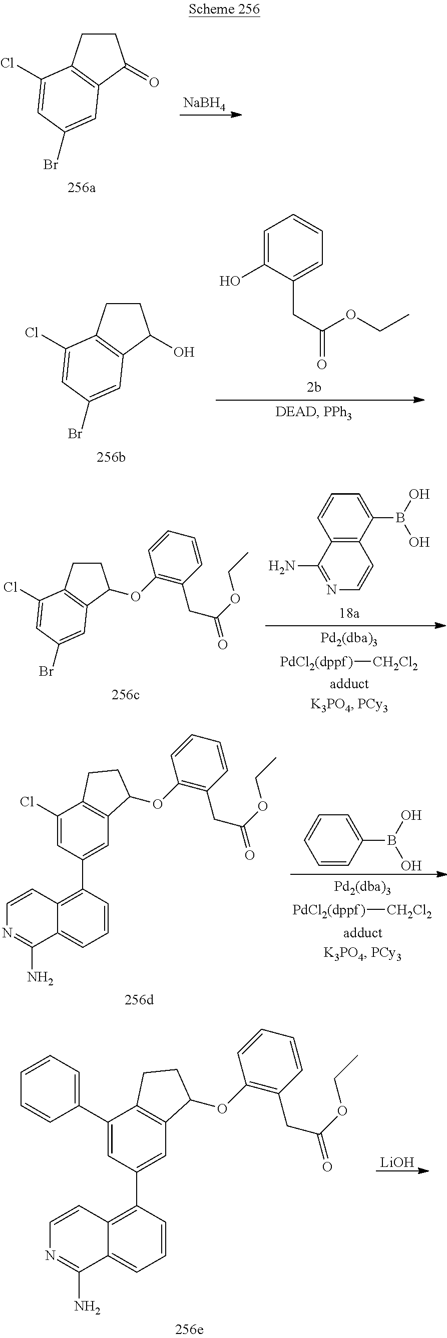

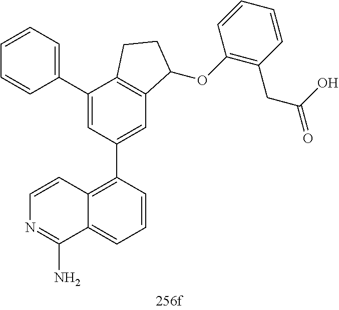 [SMILES: Nc1nccc2c(-c3cc(-c4ccccc4)c4c(c3)C(Oc3ccccc3CC(=O)O)CC4)cccc12]

### Preparation of 2-(2-((6-(1-aminoisoquinolin-5-yl)-4-phenyl-2,3-dihydro-1H-inden-1-yl)oxy)phenyl)acetic acid (256f)

### Step-1: Preparation of 6-bromo-4-chloro-2,3-dihydro-1H-inden-1-ol (256b)

Compound 256b was prepared according to the procedure reported in step-1 of scheme-205 from 6-bromo-4-chloro-2,3-dihydro-1H-inden-1-one (256a) (1 g, 4.07 mmol; CAS #1260017-17-1) in anhydrous MeOH (30 mL) using NaBH₄ (0.231 g, 6.11 mmol) to afford after workup and purification using method-X, 6-bromo-4-chloro-2,3-dihydro-1H-inden-1-ol (256b) (900 mg, 89% yield) as a white solid; ¹H NMR (300 MHz, DMSO-d₆) ¿ 7.57-7.51 (m, 1H), 7.47-7.40 (m, 1H), 5.53 (s, 1H), 5.09 (t, J=6.9 Hz, 1H), 2.96-2.81 (m, 1H), 2.77-2.59 (m, 1H), 2.44-2.29 (m, 1H), 1.88-1.71 (m, 1H).

### Step-2: Preparation of ethyl 2-(2-((6-bromo-4-chloro-2,3-dihydro-1H-inden-1-yl)oxy)phenyl)acetate (256c)

Compound 256c was prepared according to the procedure reported in step-1 of scheme-8 from 6-bromo-4-chloro-2,3-dihydro-1H-inden-1-ol (256b) (900 mg, 3.64 mmol) in THF (30 mL) using ethyl 2-(2-hydroxyphenyl)acetate (2b) (1310 mg, 7.27 mmol), PPh₃ (1907 mg, 7.27 mmol) and a solution of DEAD (1266 mg, 7.27 mmol) in THF to afford after workup and purification using method-P, ethyl 2-(2-((6-bromo-4-chloro-2,3-dihydro-1H-inden-1-yl)oxy)phenyl)acetate (256c) (400 mg, 26.9% yield) as a white oil; ¹H NMR (300 MHz, DMSO-d₆) δ 7.68 (d, J=1.7 Hz, 1H), 7.47-7.42 (m, 1H), 7.34-7.25 (m, 1H), 7.25-7.14 (m, 2H), 6.97-6.88 (m, 1H), 5.94-5.86 (m, 1H), 3.99-3.90 (m, 2H), 3.50 (s, 2H), 3.05-2.92 (m, 1H), 2.92-2.79 (m, 1H), 2.72-2.57 (m, 1H), 2.06-1.91 (m, 1H), 1.04 (t, J=7.1 Hz, 3H); MS (ES+): 431.00 & 433.00 (M+Na).

### Step-3: Preparation of ethyl 2-(2-((6-(1-aminoisoquinolin-5-yl)-4-chloro-2,3-dihydro-1H-inden-1-yl)oxy)phenyl)acetate (256d)

Compound 256d was prepared according to the procedure reported in step-5 of scheme-1 from ethyl 2-(2-((6-bromo-4-chloro-2,3-dihydro-1H-inden-1-yl)oxy)phenyl)acetate (256c) (190 mg, 0.464 mmol) in dioxane/THF (4 mL, 1:1) using (1-aminoisoquinolin-5-yl)boronic acid (18a) (87 mg, 0.464 mmol), K₃PO₄ (2 M aqueous solution, 0.928 mL, 1.855 mmol), PCy₃ (26.0 mg, 0.093 mmol), PdCl₂(dppf)-CH₂Cl₂ adduct (37.9 mg, 0.046 mmol) and Pd₂(dba)₃ (42.5 mg, 0.046 mmol) to afford after workup and purification using method-AM, ethyl 2-(2-((6-(1-aminoisoquinolin-5-yl)-4-chloro-2,3-dihydro-1H-inden-1-yl)oxy)phenyl)acetate (256d) (120 mg, 54.7% yield) as a clear oil; ¹H NMR (300 MHZ, DMSO-d₆) δ 8.24 (d, J=7.6 Hz, 1H), 7.75 (d, J=6.0 Hz, 1H), 7.56-7.53 (m, 2H), 7.46 (s, 1H), 7.32-7.29 (m, 1H), 7.29-7.24 (m, 1H), 7.23-7.18 (m, 2H), 7.00-6.87 (m, 3H), 6.71 (d, J=6.1 Hz, 1H), 5.98 (t, J=5.9 Hz, 1H), 3.86-3.59 (m, 2H), 3.47 (d, J=7.6 Hz, 2H), 3.14-3.07 (m, 1H), 3.06-2.94 (m, 1H), 2.82-2.68 (m, 1H), 2.14-1.99 (m, 1H), 0.79 (t, J=7.1 Hz, 3H); MS (ES+): 473.20 & 475.10 (M+1).

### Step-4: Preparation of ethyl 2-(2-((6-(1-aminoisoquinolin-5-yl)-4-phenyl-2,3-dihydro-1H-inden-1-yl)oxy)phenyl)acetate (256e)

Compound 256e was prepared according to the procedure reported in step-5 of scheme-1 from ethyl 2-(2-((6-(1-aminoisoquinolin-5-yl)-4-chloro-2,3-dihydro-1H-inden-1-yl)oxy)phenyl)acetate (256d) (60 mg, 0.127 mmol) in dioxane/THF (4 mL, 1:1) using phenylboronic acid (30.9 mg, 0.254 mmol), K₃PO₄ (2 M aqueous solution, 0.254 mL, 0.507 mmol), PCy₃ (7.12 mg, 0.025 mmol), PdCl₂(dppf)-CH₂Cl₂ adduct (10.36 mg, 0.013 mmol) and Pd₂(dba)₃ (11.62 mg, 0.013 mmol)) to afford after workup and purification using method-AP, ethyl 2-(2-((6-(1-aminoisoquinolin-5-yl)-4-phenyl-2,3-dihydro-1H-inden-1-yl)oxy)phenyl)acetate (256e) (30 mg, 46.0% yield) as a clear oil; MS (ES+): 515.20 (M+1).

### Step-5: Preparation of 2-(2-((6-(1-aminoisoquinolin-5-yl)-4-phenyl-2,3-dihydro-1H-inden-1-yl)oxy)phenyl)acetic acid (256f)

Compound 256f was prepared according to the procedure reported in step-2 of scheme-1 from ethyl 2-(2-((6-(1-aminoisoquinolin-5-yl)-4-phenyl-2,3-dihydro-1H-inden-1-yl)oxy)phenyl)acetate (256e) (30 mg, 0.058 mmol) in MeOH/THF (6 mL, 1:1) using a solution of LiOH·H₂O (14.68 mg, 0.350 mmol) in water (2 mL) to afford after workup and purification using method-G, 2-(2-((6-(1-aminoisoquinolin-5-yl)-4-phenyl-2,3-dihydro-1H-inden-1-yl)oxy)phenyl)acetic acid (256f) (8 mg, 28.2% yield) HCl salt as a white solid; 1H NMR (300 MHz, DMSO-d₆) δ 13.13 (s, 1H, D₂O exchangeable), 9.09 (s, 2H, D₂O) exchangeable), 8.60 (d, J=8.3 Hz, 1H), 8.06-7.94 (m, 1H), 7.94-7.79 (m, 1H), 7.66-7.57 (m, 3H), 7.55-7.45 (m, 2H), 7.45-7.37 (m, 3H), 7.32-7.24 (m, 2H), 7.24-7.17 (m, 1H), 7.07 (d, J=7.2 Hz, 1H), 6.97-6.86 (m, 1H), 5.97 (t, J=6.1 Hz, 1H), 3.48 (d, J=3.2 Hz, 2H), 3.16-3.06 (m, 2H), 2.80-2.65 (m, 1H), 2.11-1.96 (m, 1H); MS (ES+): 487.20 (M+1).

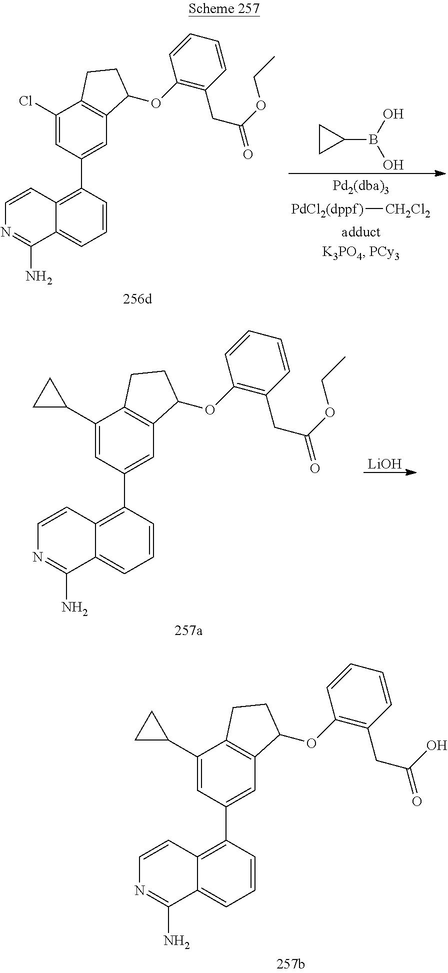

### Preparation of 2-(2-((6-(1-aminoisoquinolin-5-yl)-4-cyclopropyl-2,3-dihydro-1H-inden-1-yl)oxy)phenyl)acetic acid (257b)

### Step-1: Preparation of ethyl 2-(2-((6-(1-aminoisoquinolin-5-yl)-4-cyclopropyl-2,3-dihydro-1H-inden-1-yl)oxy)phenyl)acetate (257a)

Compound 257a was prepared according to the procedure reported in step-5 of scheme-1 from ethyl 2-(2-((6-(1-aminoisoquinolin-5-yl)-4-chloro-2,3-dihydro-1H-inden-1-yl)oxy)phenyl)acetate (256d) (60 mg, 0.127 mmol) in dioxane/THF (4 mL, 1:1) using cyclopropyl boronic acid (54.5 mg, 0.634 mmol), K₃PO₄ (2 M aqueous solution, 0.254 mL, 0.507 mmol), PCy₃ (7.12 mg, 0.025 mmol), PdCl₂(dppf)-CH₂Cl₂ adduct (10.36 mg, 0.013 mmol) and Pd₂(dba)₃ (11.62 mg, 0.013 mmol) to afford after workup and purification using method-AP, ethyl 2-(2-((6-(1-aminoisoquinolin-5-yl)-4-cyclopropyl-2,3-dihydro-1H-inden-1-yl)oxy)phenyl)acetate (257a) (30 mg, 49.4% yield) as a clear oil; MS (ES+): 479.20 (M+1).

### Step-2: Preparation of 2-(2-((6-(1-aminoisoquinolin-5-yl)-4-cyclopropyl-2,3-dihydro-1H-inden-1-yl)oxy)phenyl)acetic acid (257b)

Compound 257b was prepared according to the procedure reported in step-2 of scheme-1 from ethyl 2-(2-((6-(1-aminoisoquinolin-5-yl)-4-cyclopropyl-2,3-dihydro-1H-inden-1-yl)oxy)phenyl)acetate (257a) (30 mg, 0.063 mmol) in MeOH/THF (6 mL, 1:1) using a solution of LiOH·H₂O (15.78 mg, 0.376 mmol) in water (2 mL) to afford after workup and purification using method-G, 2-(2-((6-(1-aminoisoquinolin-5-yl)-4-cyclopropyl-2,3-dihydro-1H-inden-1-yl)oxy)phenyl)acetic acid (257b) (12 mg, 42.5% yield) HCl salt as a white solid; ¹H NMR (300 MHz, DMSO-d₆) δ 13.14 (s, 1H, D₂O exchangeable), 12.01 (s, 1H, D₂O exchangeable), 9.10 (s, 2H, D₂O exchangeable), 8.57 (d, J=7.8 Hz, 1H), 7.91-7.77 (m, 2H), 7.59 (d, J=7.3 Hz, 1H), 7.30-7.15 (m, 4H), 6.97-6.89 (m, 2H), 6.89-6.83 (m, 1H), 5.88 (t, J=6.0 Hz, 1H), 3.44 (d, J=3.2 Hz, 2H), 3.24-3.14 (m, 1H), 3.08-2.96 (m, 1H), 2.84-2.68 (m, 1H), 2.15-1.93 (m, 2H), 1.03-0.94 (m, 2H), 0.81-0.69 (m, 2H); MS (ES+): 451.15 (M+1); (ES−): 449.10 (M−1).

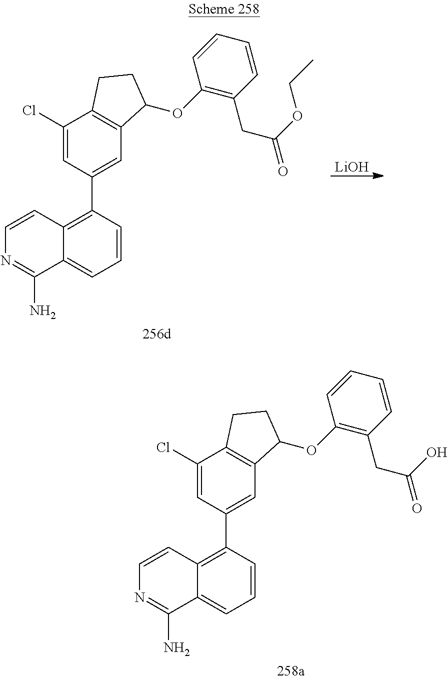

### Preparation of 2-(2-((6-(1-aminoisoquinolin-5-yl)-4-chloro-2,3-dihydro-1H-inden-1-yl)oxy)phenyl)acetic acid (258a)

Compound 258a was prepared according to the procedure reported in step-2 of scheme-1 from ethyl 2-(2-((6-(1-aminoisoquinolin-5-yl)-4-chloro-2,3-dihydro-1H-inden-1-yl)oxy)phenyl)acetate (256d) (50 mg, 0.106 mmol) in MeOH/THF (6 mL, 1:1) using a solution of LiOH·H₂O (26.6 mg, 0.634 mmol) in water (2 mL) to afford after workup and purification using method-G, 2-(2-((6-(1-aminoisoquinolin-5-yl)-4-chloro-2,3-dihydro-1H-inden-1-yl)oxy)phenyl)acetic acid (258a) (15 mg, 31.9% yield) HCl salt as a white solid; ¹H NMR (300 MHz, DMSO-d₆) δ 13.29 (s, 1H, D₂O exchangeable), 12.01 (s, 1H, D₂O exchangeable), 9.12 (s, 2H, D₂O exchangeable), 8.62 (d, J=8.2 Hz, 1H), 7.95-7.88 (m, 1H), 7.84 (t, J=7.8 Hz, 1H), 7.62 (d, J=7.2 Hz, 1H), 7.49 (d, 1H), 7.36 (s, 1H), 7.31-7.15 (m, 3H), 6.96-6.86 (m, 2H), 5.96 (t, J=5.9 Hz, 1H), 3.46-3.42 (m, 2H), 3.20-3.10 (m. 1H), 3.07-2.91 (m, 1H), 2.84-2.69 (m, 1H), 2.17-2.02 (m, 1H); MS (ES+): 445.10 & 447.10 (M+1); (ES−): 443.05 &445.10 (M−1).

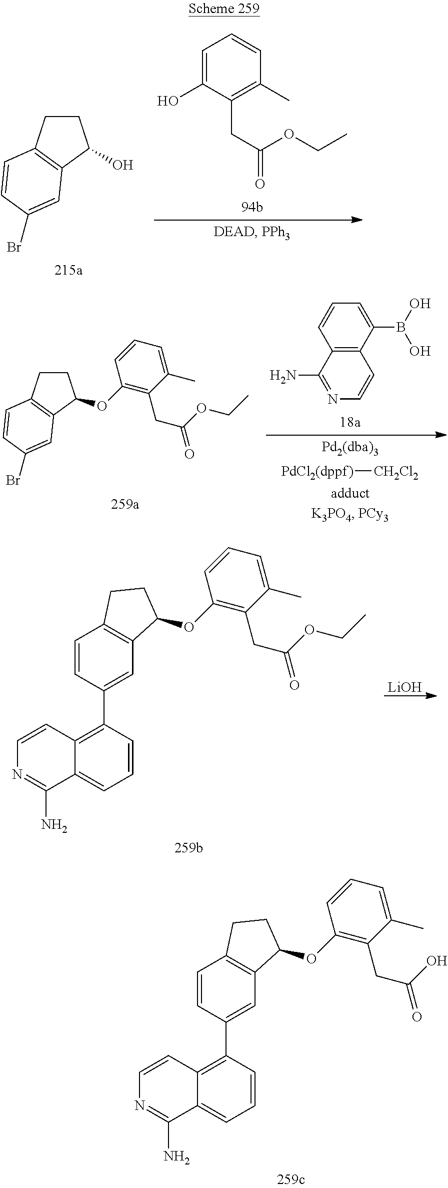

### Preparation of (R)-2-(2-((6-(1-aminoisoquinolin-5-yl)-2,3-dihydro-1H-inden-1-yl)oxy)-6-methylphenyl)acetic acid (259c)

### Step-1: Preparation of (R)-ethyl 2-(2-((6-bromo-2,3-dihydro-1H-inden-1-yl)oxy)-6-methylphenyl)acetate (259a)

Compound 259a was prepared according to the procedure reported in step-1 of scheme-8 from (S)-6-bromo-2,3-dihydro-1H-inden-1-ol (215a) (500 mg, 2.347 mmol; CAS #1096537-29-9) in DCM (10 mL) using ethyl 2-(2-hydroxy-6-methylphenyl)acetate (94b) (593 mg, 3.05 mmol), PPh₃ (1231 mg, 4.69 mmol) and a solution of DEAD (817 mg, 4.69 mmol) in DCM to afford after workup and purification using method-L, (R)-ethyl 2-(2-((6-bromo-2,3-dihydro-1H-inden-1-yl)oxy)-6-methylphenyl)acetate (259a) (250 mg, 27.4% yield) as a yellow oil; MS (ES+): 411.00 & 413.05 (M+Na).

### Step-2: Preparation of (R)-ethyl 2-(2-((6-(1-aminoisoquinolin-5-yl)-2,3-dihydro-1H-inden-1-yl)oxy)-6-methylphenyl)acetate (259b)

Compound 259b was prepared according to the procedure reported in step-5 of scheme-1 from (R)-ethyl 2-(2-((6-bromo-2,3-dihydro-1H-inden-1-yl)oxy)-6-methylphenyl)acetate (259a) (250 mg, 0.642 mmol) in dioxane/THF (4 mL, 1:1) using (1-aminoisoquinolin-5-yl)boronic acid (18a) (181 mg, 0.963 mmol), K₃PO₄ (2 M aqueous solution, 1.284 mL, 2.57 mmol), PCy₃ (36.0 mg, 0.128 mmol), PdCl₂(dppf)-CH₂Cl₂ adduct (52.4 mg, 0.064 mmol) and Pd₂(dba)₃ (58.8 mg, 0.064 mmol) to afford after workup and purification using method-Y, (R)-ethyl 2-(2-((6-(1-aminoisoquinolin-5-yl)-2,3-dihydro-1H-inden-1-yl)oxy)-6-methylphenyl)acetate (259b) (200 mg, 68.8% yield) as a clear oil; ¹H NMR (300 MHz, DMSO-d₆) δ 8.26-8.17 (m, 1H), 7.72 (d, J=6.1 Hz, 1H), 7.56-7.42 (m, 4H), 7.40-7.31 (m, 2H), 7.15 (t, J=7.8 Hz, 1H), 7.07 (d, J=8.2 Hz, 1H), 6.98 (s, 2H), 6.81-6.75 (m, 1H), 5.87 (t, J=5.7 Hz, 1H), 3.83-3.61 (m, 2H), 3.52 (d, J=5.1 Hz, 2H), 3.18-3.06 (m, 1H), 3.05-2.90 (m, 1H), 2.77-2.62 (m, 1H), 2.19 (s, 3H), 2.10-1.96 (m, 1H), 0.82 (t, J=7.1 Hz, 3H); MS (ES+): 453.20 (M+1).

### Step-3: Preparation of (R)-2-(2-((6-(1-aminoisoquinolin-5-yl)-2,3-dihydro-1H-inden-1-yl)oxy)-6-methylphenyl)acetic acid (259c)

Compound 259c was prepared according to the procedure reported step-2 of scheme-1 from (R)-ethyl 2-(2-(6-(1-aminoisoquinolin-5-yl)-2,3-dihydro-1H-inden-1-yloxy)-6-methylphenyl)acetate (259b) (200 mg, 0.442 mmol) in MeOH/THF (6 mL, 1:1) using a solution of LiOH·H₂O (111 mg, 2.65 mmol) in water (2 mL) to afford after workup and purification using method-G. (R)-2-(2-((6-(1-aminoisoquinolin-5-yl)-2,3-dihydro-1H-inden-1-yl)oxy)-6-methylphenyl)acetic acid (259c) (50 mg, 26.7% yield) HCl salt as a white solid; ¹H NMR (300 MHz, DMSO-d₆) δ 13.43 (s, 1H, D₂O exchangeable), 12.05 (s, 1H, D₂O exchangeable), 9.21 (s, 2H, D₂O exchangeable), 8.62 (d, J=8.0 Hz, 1H), 7.92-7.78 (m, 2H), 7.61 (d, J=7.3 Hz, 1H), 7.55-7.44 (m, 1H), 7.43-7.34 (m, 2H), 7.20-7.10 (m, 1H), 7.10-7.03 (m, 1H), 6.94 (d, J=7.3 Hz, 1H), 6.80 (d, J=7.3 Hz, 1H), 5.86 (t, J=5.8 Hz, 1H), 3.50 (d, J=3.5 Hz, 2H), 3.20-3.07 (m, 1H), 3.05-2.90 (m, 1H), 2.78-2.62 (m, 1H), 2.20 (s, 3H), 2.14-1.96 (m, 1H); MS (ES+): 425.20 (M+1); (ES−): 423.10 (M−1).

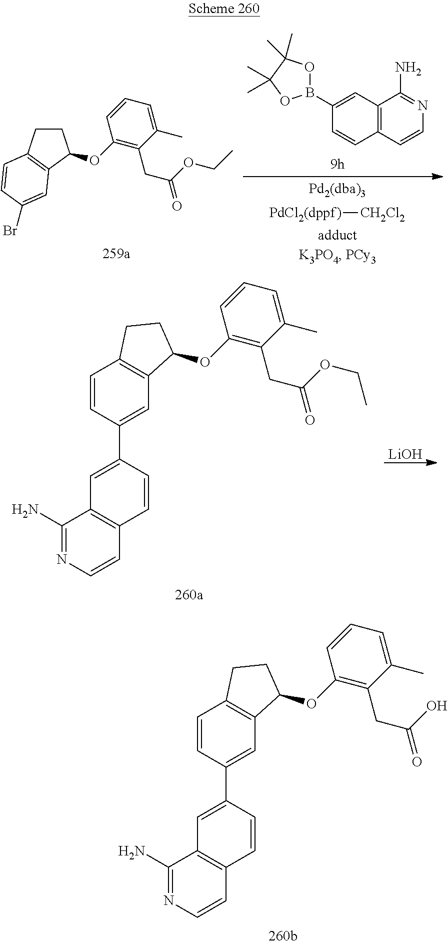

### Preparation of (R)-2-(2-((6-(1-aminoisoquinolin-7-yl)-2,3-dihydro-1H-inden-1-yl)oxy)-6-methylphenyl)acetic acid (260b)

### Step-1: Preparation of (R)-ethyl 2-(2-((6-(1-aminoisoquinolin-7-yl)-2,3-dihydro-1H-inden-1-yl)oxy)-6-methylphenyl)acetate (260a)

Compound 260a was prepared according to the procedure reported in step-5 of scheme-1 from (R)-ethyl 2-(2-((6-bromo-2,3-dihydro-1H-inden-1-yl)oxy)-6-methylphenyl)acetate (259a) (250 mg, 0.642 mmol) in dioxane/THF (4 mL, 1:1) using 7-(4,4,5,5-tetramethyl-1,3,2-dioxaborolan-2-yl)isoquinolin-1-amine (9h) (260 mg, 0.963 mmol), K₃PO₄ (2 M aqueous solution, 1.284 mL, 2.57 mmol), PCy₃ (36.0 mg, 0.128 mmol), PdCl₂(dppf)-CH₂Cl₂ adduct (52.4 mg, 0.064 mmol) and Pd₂(dba)₃ (58.8 mg, 0.064 mmol) to afford after workup and purification using method-Y. (R)-ethyl 2-(2-((6-(1-aminoisoquinolin-7-yl)-2,3-dihydro-1H-inden-1-yl)oxy)-6-methylphenyl)acetate (260a) (200 mg, 68.8% yield) as a clear oil; ¹H NMR (300 MHz, DMSO-d₆) δ 8.52 (s, 1H), 7.99-7.90 (m, 1H), 7.82-7.75 (m, 3H), 7.50-7.43 (m, 1H), 7.22-7.14 (m, 1H), 7.14-6.97 (m, 4H), 6.97-6.90 (m, 1H), 6.85-6.78 (m, 1H), 5.93-5.78 (m, 1H), 3.82 (q, J=7.3 Hz, 2H), 3.55 (s, 2H), 3.15-3.02 (m, 1H), 3.02-2.88 (m, 1H), 2.74-2.61 (m, 1H), 2.24-2.17 (m, 3H), 2.05-1.95 (m, 1H), 0.82 (t, J=7.4 Hz, 3H); MS (ES+): 453.20 (M+1).

### Step-2: Preparation of (R)-2-(2-((6-(1-aminoisoquinolin-7-yl)-2,3-dihydro-1H-inden-1-yl)oxy)-6-methylphenyl)acetic acid (260b)

Compound 260b was prepared according to the procedure reported step-2 of scheme-1 from (R)-ethyl 2-(2-((6-(1-aminoisoquinolin-7-yl)-2,3-dihydro-1H-inden-1-yl)oxy)-6-methylphenyl)acetate (260a) (200 mg, 0.442 mmol) in MeOH/THF (6 mL, 1:1) using a solution of LiOH·H₂O (111 mg, 2.65 mmol) in water (2 mL) to afford after workup and purification using method-G, (R)-2-(2-((6-(1-aminoisoquinolin-7-yl)-2,3-dihydro-1H-inden-1-yl)oxy)-6-methylphenyl)acetic acid (260b) (55 mg, 29.3% yield) HCl salt as a white solid; ¹H NMR (300 MHz, DMSO-d₆) δ 13.24 (s, 1H, DO exchangeable), 12.21 (s, 1H, D₂O exchangeable), 9.05 (s, 2H, D₂O exchangeable), 8.88 (d, 1H), 8.28 (dd, 1H), 8.02 (d, J=8.6 Hz, 1H), 7.92-7.82 (m, 2H), 7.70 (d, J=6.9 Hz, 1H), 7.55-7.46 (m, 1H), 7.25 (d, J=6.9 Hz, 1H), 7.22-7.14 (m, 1H), 7.14-7.06 (m, 1H), 6.82 (d, J=7.3 Hz, 1H), 5.88 (t, J=5.7 Hz, 1H), 3.53 (d, J=3.0 Hz, 2H), 3.18-3.03 (m, 1H), 3.03-2.86 (m, 1H), 2.73-2.58 (m, 1H), 2.22 (s, 3H), 2.09-1.94 (m, 1H); MS (ES+): 425.20 (M+1); (ES−): 423.10 (M−1).

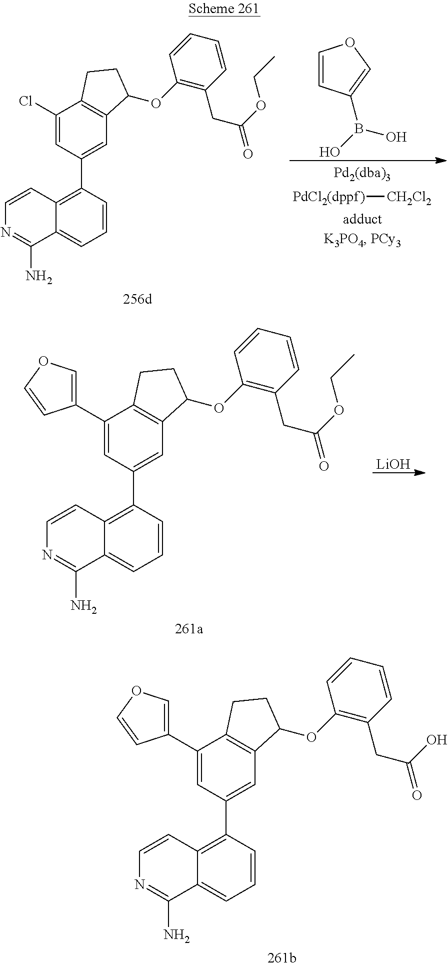

### Preparation of 2-(2-((6-(1-aminoisoquinolin-5-yl)-4-(furan-3-yl)-2,3-dihydro-1H-inden-1-yl)oxy)phenyl)acetic acid (261b)

### Step-1: Preparation of ethyl 2-(2-((6-(1-aminoisoquinolin-5-yl)-4-(furan-3-yl)-2,3-dihydro-1H-inden-1-yl)oxy)phenyl)acetate (261a)

Compound 261a was prepared according to the procedure reported in step-5 of scheme-1 from ethyl 2-(2-((6-(1-aminoisoquinolin-5-yl)-4-chloro-2,3-dihydro-1H-inden-1-yl)oxy)phenyl)acetate (256d) (60 mg, 0.127 mmol) in dioxane/THF (4 mL, 1:1) using furan-3-ylboronic acid (42.6 mg, 0.381 mmol), K₃PO₄ (2 M aqueous solution, 0.254 mL, 0.507 mmol), PCy₃ (7.12 mg, 0.025 mmol) PdCl₂(dppf)-CH₂Cl₂ adduct (10.6 mg, 0.013 mmol) and Pd₂(dba)₃ (11.62 mg, 0.013 mmol) to afford after workup and purification using method-AP, ethyl 2-(2-((6-(1-aminoisoquinolin-5-yl)-4-(furan-3-yl)-2,3-dihydro-1H-inden-1-yl)oxy)phenyl)acetate (261a) (50 mg, 78% yield) as a clear oil; MS (ES+): 505.20 (M+1).

### Step-2: Preparation of 2-(2-((6-(1-aminoisoquinolin-5-yl)-4-(furan-3-yl)-2,3-dihydro-1H-inden-1-yl)oxy)phenyl)acetic acid (261b)

Compound 261b was prepared according to the procedure reported step-2 of scheme-1 from ethyl 2-(2-((6-(1-aminoisoquinolin-5-yl)-4-(furan-3-yl)-2,3-dihydro-1H-inden-1-yl)oxy)phenyl)acetate (261a) (50 mg, 0.099 mmol) in MeOH/THF (6 mL, 1:1) using a solution of LiOH·H₂O (24.95 mg, 0.595 mmol) in water (2 mL) to afford after workup and purification using method-G, 2-(2-((6-(1-aminoisoquinolin-5-yl)-4-(furan-3-yl)-2,3-dihydro-1H-inden-1-yl)oxy)phenyl)acetic acid (261b) (8 mg, 16.94% yield) HCl salt as a white solid; ¹H NMR (300 MHz, DMSO-d₆) δ 13.10 (s, 1H, D₂O exchangeable), 12.02 (s, 1H, DO exchangeable), 8.99 (s, 2H, D₂O exchangeable), 8.58 (d, J=8.3 Hz, 1H), 8.17-8.09 (m, 1H), 8.01-7.91 (m, 1H), 7.91-7.77 (m, 2H), 7.66-7.57 (m, 2H), 7.38-7.15 (m, 4H), 7.05-6.96 (m, 2H), 6.96-6.86 (m, 1H), 5.94 (t, J=6.0 Hz, 1H), 3.46 (m, 2H), 3.31-3.02 (m, 2H), 2.89-2.69 (m, 1H), 2.15-2.05 (m, 1H); (ES+): 477.10 (M+1).

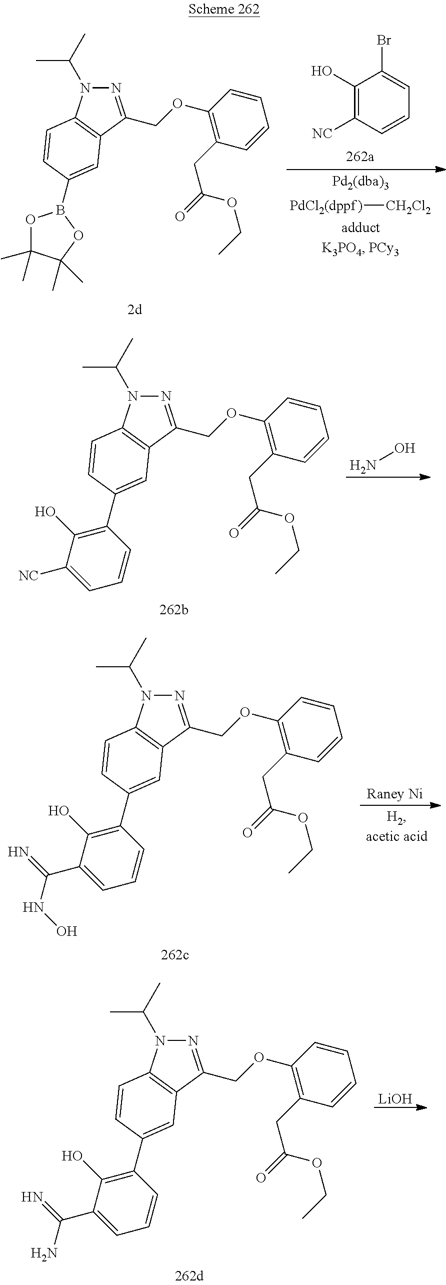

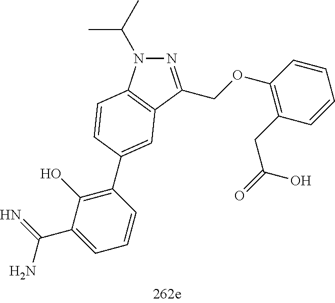 [SMILES: CC(C)n1nc(COc2ccccc2CC(=O)O)c2cc(-c3cccc(C(=N)N)c3O)ccc21]

### Preparation of 2-(2-((5-(3-carbamimidoyl-2-hydroxyphenyl)-1-isopropyl-1H-indazol-3-yl)methoxy)phenyl)acetic acid (262e)

### Step-1: Preparation of ethyl 2-(2-((5-(3-cyano-2-hydroxyphenyl)-1-isopropyl-1H-indazol-3-yl)methoxy)phenyl)acetate (262b)

Compound 262b was prepared according to the procedure reported in step-S of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (600 mg, 1.254 mmol) in dioxane/THF (6 mL, 1:1) using 3-bromo-2-hydroxybenzonitrile (262a) (323 mg, 1.630 mmol; CAS #13073-28-4), K₃PO₄ (2 M aqueous solution, 2.508 mL, 5.02 mmol), PCy₃ (70.3 mg, 0.251 mmol), PdCl₂(dppf)-CH₂Cl₂ adduct (102 mg, 0.125 mmol) and Pd₂(dba)₃ (115 mg, 0.125 mmol) to afford after workup and purification using method-P, ethyl 2-(2-((5-(3-cyano-2-hydroxyphenyl)-1-isopropyl-1H-indazol-3-yl)methoxy)phenyl)acetate (262b) (370 mg, 62.8% yield) as a clear oil; ¹H NMR (300 MHz, DMSO-d₆) δ 10.17 (s, 1H), 7.85-7.76 (m, 2H), 7.66-7.55 (m, 2H), 7.55-7.47 (m, 1H), 7.27 (d, J=3.8 Hz, 2H), 7.18 (d, J=7.3 Hz, 1H), 7.08 (t, J=7.7 Hz, 1H), 6.94-6.86 (m, 1H), 5.41 (s, 2H), 5.03 (h, J=6.6 Hz, 1H), 3.69 (q, J=7.1 Hz, 2H), 3.53 (s, 2H), 1.52 (d, J=6.5 Hz, 6H), 0.86 (t, J=7.1 Hz, 3H); MS (ES+): 470.20 (M+1); 492.20 (M+Na); MS (ES−): 468.20 (M−1).

### Step-2: Preparation of ethyl 2-(2-((5-(2-hydroxy-3-(N-hydroxycarbamimidoyl)phenyl)-1-isopropyl-1H-indazol-3-yl)methoxy)phenyl)acetate (262c)

Compound 262c was prepared according to the procedure reported in step-5 of scheme-23 from ethyl 2-(2-((5-(3-cyano-2-hydroxyphenyl)-1-isopropyl-1H-indazol-3-yl)methoxy)phenyl)acetate (262b) (370 mg, 0.788 mmol) in EtOH (10 mL) using hydroxylamine (0.521 mL, 7.88 mmol) to afford after workup and purification using method-E, ethyl 2-(2-((5-(2-hydroxy-3-(N-hydroxycarbamimidoyl)phenyl)-1-isopropyl-1H-indazol-3-yl)methoxy)phenyl)acetate (262c) (250 mg, 0.497 mmol, 63.1% yield) as a white solid; MS (ES+): 503.20 (M+1); 525.20 (M+Na).

### Step-3: Preparation of ethyl 2-(2-((5-(3-carbamimidoyl-2-hydroxyphenyl)-1-isopropyl-1H-indazol-3-yl)methoxy)phenyl)acetate (262d)

Compound 262d was prepared according to the procedure reported in step-1 of scheme-24 from ethyl 2-(2-((5-(2-hydroxy-3-(N-hydroxycarbamimidoyl)phenyl)-1-isopropyl-1H-indazol-3-yl)methoxy)phenyl)acetate (262c) (250 mg, 0.497 mmol) in EtOH (10 mL) using AcOH (0.028 mL, 0.497 mmol), Raney Nickel (0.497 mmol) and hydrogen (1 atm) to afford after workup and purification using method-AI, ethyl 2-(2-((5-(3-carbamimidoyl-2-hydroxyphenyl)-1-isopropyl-1H-indazol-3-yl)methoxy)phenyl)acetate (262d) (200 mg, 83% yield) as a colorless oil; MS (ES+): 487.20 (M+1); (ES−): 485.20 (M−1).

### Step-4: Preparation of 2-(2-((5-(3-carbamimidoyl-2-hydroxyphenyl)-1-isopropyl-1H-indazol-3-yl)methoxy)phenyl)acetic acid (262e)

Compound 262e was prepared according to the procedure reported step-2 of scheme-1 from ethyl 2-(2-((5-(3-carbamimidoyl-2-hydroxyphenyl)-1-isopropyl-1H-indazol-3-yl)methoxy)phenyl)acetate (262d) (200 mg, 0.411 mmol) in MeOH/THF (6 mL, 1:1) using a solution of LiOH·H₂O (103 mg, 2.466 mmol) in water (2 mL) to afford after workup and purification using method-G, 2-(2-((5-(3-carbamimidoyl-2-hydroxyphenyl)-1-isopropyl-1H-indazol-3-yl)methoxy)phenyl)acetic acid (262e) (75 mg, 39.8% yield) HCl salt as a white solid; ¹H NMR (300 MHz, DMSO-d₆) δ 12.11 (s, 1H, D₂O exchangeable), 9.61 (s, 1H, D₂O exchangeable), 9.22 (s, 2H, D₂O exchangeable), 9.16 (s, 2H, D₂O exchangeable), 7.90-7.86 (m, 1H), 7.84-7.76 (m, 1H), 7.60-7.50 (m, 2H), 7.43 (dd, J=7.7, 1.7 Hz, 1H), 7.32-7.21 (m, 2H), 7.21-7.15 (m, 1H), 7.11 (t, J=7.7 Hz, 1H), 6.95-6.85 (m, 1H), 5.43 (s, 2H), 5.16-4.89 (m, 1H), 3.51 (s, 2H), 1.52 (d, J=6.6 Hz, 6H); MS (ES+): 459.20 (M+1); (ES−): 457.10 (M−1).

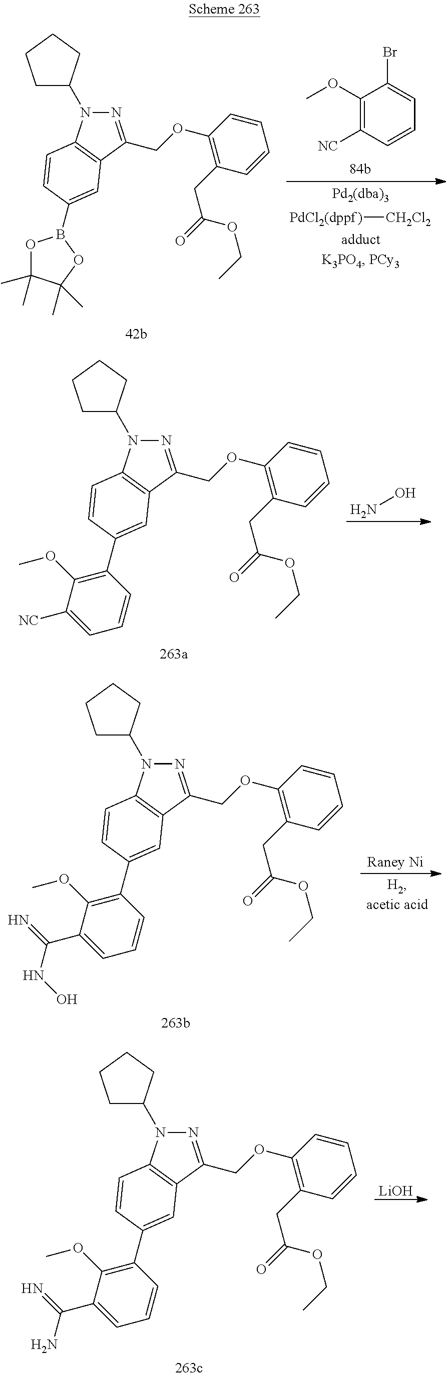

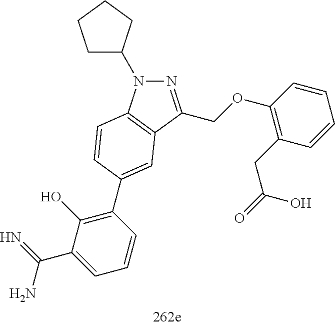 [SMILES: N=C(N)c1cccc(-c2ccc3c(c2)c(COc2ccccc2CC(=O)O)nn3C2CCCC2)c1O]

### Preparation of 2-(2-((5-(3-carbamimidoyl-2-methoxyphenyl)-1-cyclopentyl-1H-indazol-3-yl)methoxy)phenyl)acetic acid (263d)

### Step-1: Preparation of ethyl 2-(2-((5-(3-cyano-2-methoxyphenyl)-1-cyclopentyl-1H-indazol-3-yl)methoxy)phenyl)acetate (263a)

Compound 263a was prepared according to the procedure reported in step-5 of scheme-1 from ethyl 2-(2-((1-cyclopentyl-5-(4,4,5,5-tetramethyl-1,3,2-dioxaborolan-2-yl)-1H-indazol-3-yl)methoxy)phenyl)acetate (42b) (240 mg, 0.476 mmol) in dioxane/THF (4 mL, 1:1) using 3-bromo-2-methoxybenzonitrile (84b) (151 mg, 0.714 mmol), K₃PO₄ (2 M aqueous solution, 0.952 mL, 1.903 mmol), PCy₃ (26.7 mg, 0.095 mmol), PdCl₂(dppf)-CH₂Cl₂ adduct (38.9 mg, 0.048 mmol) and Pd₂(dba)₃ (43.6 mg, 0.048 mmol) to afford after workup and purification using method-P, ethyl 2-(2-((5-(3-cyano-2-methoxyphenyl)-1-cyclopentyl-1H-indazol-3-yl)methoxy)phenyl)acetate (263a) (200 mg, 82% yield) as a clear oil; MS (ES+): 510.20 (M+1).

### Step-2: Preparation of ethyl 2-(2-((1-cyclopentyl-5-(3-(N-hydroxycarbamimidoyl)-2-methoxyphenyl)-1H-indazol-3-yl)methoxy)phenyl)acetate (263b)

Compound 263b was prepared according to the procedure reported in step-5 of scheme-23 from ethyl 2-(2-((5-(3-cyano-2-methoxyphenyl)-1-cyclopentyl-1H-indazol-3-yl)methoxy)phenyl)acetate (263a) (200 mg, 0.392 mmol) in EtOH (10 mL) using hydroxylamine (0.259 mL, 3.92 mmol) to afford after workup and purification using method-E, ethyl 2-(2-((1-cyclopentyl-5-(3-(N-hydroxycarbamimidoyl)-2-methoxyphenyl)-1H-indazol-3-yl)methoxy)phenyl)acetate (263b) (130 mg, 61.0% yield) as a white solid; MS (ES+): 543.25 (M+1).

### Step-3: Preparation of ethyl 2-(2-((5-(3-carbamimidoyl-2-methoxyphenyl)-1-cyclopentyl-1H-indazol-3-yl)methoxy)phenyl)acetate (263c)

Compound 263c was prepared according to the procedure reported in step-1 of scheme-24 from ethyl 2-(2-((1-cyclopentyl-5-(3-(N-hydroxycarbamimidoyl)-2-methoxyphenyl)-1H-indazol-3-yl)methoxy)phenyl)acetate (263b) (130 mg, 0.240 mmol) in EtOH (10 mL) using AcOH (0.014 mL, 0.240 mmol), Raney Nickel (0.240 mmol) and hydrogen (1 atm) for 16 h to afford after workup and purification using method-AI, ethyl 2-(2-((5-(3-carbamimidoyl-2-methoxyphenyl)-1-cyclopentyl-1H-indazol-3-yl)methoxy)phenyl)acetate (263c) (100 mg, 79% yield) as a colorless oil; MS (ES+): 527.25 (M+1).

### Step-4: Preparation of 2-(2-((5-(3-carbamimidoyl-2-methoxyphenyl)-1-cyclopentyl-1H-indazol-3-yl)methoxy)phenyl)acetic acid (263d)

Compound 263d was prepared according to the procedure reported step-2 of scheme-1 from ethyl 2-(2-((5-(3-carbamimidoyl-2-methoxyphenyl)-1-cyclopentyl-1H-indazol-3-yl)methoxy)phenyl)acetate (263c) (100 mg, 0.190 mmol) in MeOH/THF (6 mL, 1:1) using a solution of LiOH·H₂O (47.8 mg, 1.139 mmol) in water (2 mL) to afford after workup and purification using method-G, 2-(2-((5-(3-carbamimidoyl-2-methoxyphenyl)-1-cyclopentyl-1H-indazol-3-yl)methoxy)phenyl)acetic acid (263d) (65 mg, 68.7% yield) HCl salt as a white solid; ¹H NMR (300 MHz, DMSO-d₆) δ 12.09 (s, 1H, D₂O exchangeable), 9.40 (s, 2H, D₂O exchangeable), 9.35 (s, 2H, D₂O exchangeable), 8.00-7.94 (m, 1H), 7.83 (d, J=8.8 Hz, 1H), 7.69 (dd, J=7.7, 1.8 Hz, 1H), 7.63 (dd, J=8.8, 1.6 Hz, 1H), 7.53 (dd, J=7.7, 1.7 Hz, 1H), 7.40 (t, J=7.7 Hz, 1H), 7.31-7.23 (m, 2H), 7.23-7.16 (m, 1H), 7.01-6.79 (m, 1H), 5.44 (s, 2H), 5.29-5.10 (m, 1H), 3.51 (s, 2H), 3.45 (s, 2H), 3.38 (s, 3H), 2.24-2.10 (m, 2H), 2.10-1.98 (m, 2H), 1.98-1.83 (m, 2H), 1.80-1.60 (m, 2H); MS (ES+): 499.20 (M+1); (ES−): 497.10 (M−1).

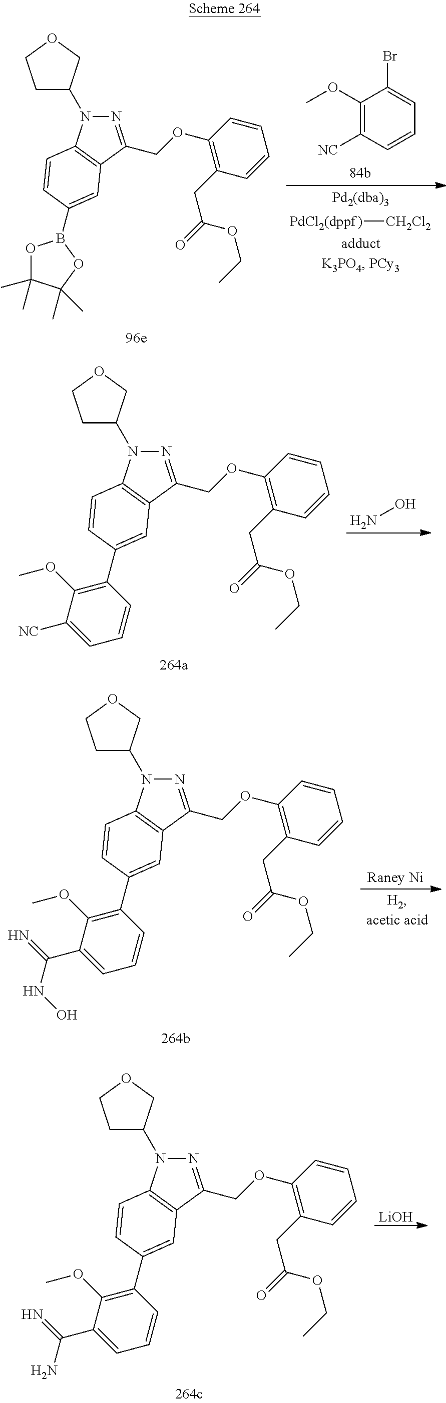

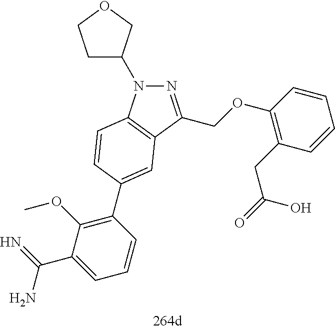 [SMILES: COc1c(C(=N)N)cccc1-c1ccc2c(c1)c(COc1ccccc1CC(=O)O)nn2C1CCOC1]

### Preparation of 2-(2-((5-(3-carbamimidoyl-2-methoxyphenyl)-1-(tetrahydrofuran-3-yl)-1H-indazol-3-yl)methoxy)phenyl)acetic acid (264d)

### Step-1: Preparation of ethyl 2-(2-((5-(3-cyano-2-methoxyphenyl)-1-(tetrahydrofuran-3-yl)-1H-indazol-3-yl)methoxy)phenyl)acetate (264a)

Compound 264a was prepared according to the procedure reported in step-5 of scheme-1 from ethyl 2-(2-((1-(tetrahydrofuran-3-yl)-5-(4,4,5,5-tetramethyl-1,3,2-dioxaborolan-2-yl)-1H-indazol-3-yl)methoxy)phenyl)acetate (96e) (800 mg, 1.580 mmol) in dioxane/THF (8 mL, 1:1) using 3-bromo-2-methoxybenzonitrile (84b) (502 mg, 2.370 mmol), K₃PO₄ (2 M aqueous solution, 3.16 mL, 6.32 mmol), PCy₃ (89 mg, 0.316 mmol), PdCl₂(dppf)-CH₂Cl₂ adduct (129 mg, 0.158 mmol) and Pd₂(dba)₃ (145 mg, 0.158 mmol) to afford after workup and purification using method-P, ethyl 2-(2-((5-(3-cyano-2-methoxyphenyl)-1-(tetrahydrofuran-3-yl)-1H-indazol-3-yl)methoxy)phenyl)acetate (264a) (470 mg, 58.2% yield) as a clear oil; MS (ES+): 512.20 (M+1).

### Step-2: Preparation of ethyl 2-(2-((5-(3-(N-hydroxycarbamimidoyl)-2-methoxyphenyl)-1-(tetrahydrofuran-3-yl)-1H-indazol-3-yl)methoxy)phenyl)acetate (264b)

Compound 264b was prepared according to the procedure reported in step-5 of scheme-23 from ethyl 2-(2-((5-(3-cyano-2-methoxyphenyl)-1-(tetrahydrofuran-3-yl)-1H-indazol-3-yl)methoxy)phenyl)acetate (264a) (470 mg, 0.919 mmol) in EtOH (10 mL) using hydroxylamine (0.607 mL, 9.19 mmol) to afford after workup and purification using method-E, ethyl 2-(2-((5-(3-(N-hydroxycarbamimidoyl)-2-methoxyphenyl)-1-(tetrahydrofuran-3-yl)-1H-indazol-3-yl)methoxy)phenyl)acetate (264b) (450 mg, 90% yield) as a white solid; MS (ES+): 545.25 (M+1).

### Step-3: Preparation of ethyl 2-(2-((5-(3-carbamimidoyl-2-methoxyphenyl)-1-(tetrahydrofuran-3-yl)-1H-indazol-3-yl)methoxy)phenyl)acetate (264c)

Compound 264c was prepared according to the procedure reported in step-1 of scheme-24 from ethyl 2-(2-((5-(3-(N-hydroxycarbamimidoyl)-2-methoxyphenyl)-1-(tetrahydrofuran-3-yl)-1H-indazol-3-yl)methoxy)phenyl)acetate (264b) (450 mg, 0.826 mmol) in EtOH (10 mL) using AcOH (0.047 mL, 0.826 mmol), Raney Nickel (0.826 mmol) and hydrogen (1 atm) to afford after workup and purification using method-AI, ethyl 2-(2-((5-(3-carbamimidoyl-2-methoxyphenyl)-1-(tetrahydrofuran-3-yl)-1H-indazol-3-yl)methoxy)phenyl)acetate (264c) (400 mg, 92% yield) as a colorless oil; MS (ES+): 529.25 (M+1).

### Step-4: Preparation of 2-(2-((5-(3-carbamimidoyl-2-methoxyphenyl)-1-(tetrahydrofuran-3-yl)-1H-indazol-3-yl)methoxy)phenyl)acetic acid (264d)

Compound 264d was prepared according to the procedure reported step-2 of scheme-1 from ethyl 2-(2-((5-(3-carbamimidoyl-2-methoxyphenyl)-1-(tetrahydrofuran-3-yl)-1H-indazol-3-yl)methoxy)phenyl)acetate (264c) (400 mg, 0.757 mmol) in MeOH/THF (6 mL, 1:1) using a solution of LiOH·H₂O (191 mg, 4.54 mmol) in water (2 mL) to afford after workup and purification using method-G, 2-(2-((5-(3-carbamimidoyl-2-methoxyphenyl)-1-(tetrahydrofuran-3-yl)-1H-indazol-3-yl)methoxy)phenyl)acetic acid (264d) (95 mg, 25.08% yield) HCl salt as a white solid; ¹H NMR (300 MHz, DMSO-d₆) δ 12.09 (s, 1H, D₂O exchangeable), 9.40 (s, 2H, D₂O exchangeable), 9.35 (s, 2H, D₂O exchangeable), 7.99 (d, 1H), 7.87 (d, J=8.8 Hz, 1H), 7.74-7.61 (m, 2H), 7.53 (dd. J=7.7, 1.7 Hz, 1H), 7.44-7.31 (m, 1H), 7.31-7.23 (m, 2H), 7.23-7.16 (m, 1H), 6.97-6.83 (m, 1H), 5.63-5.50 (m, 1H), 5.45 (s, 2H), 4.22-4.04 (m, 2H), 4.04-3.84 (m, 2H), 3.51 (s, 2H), 3.37 (s, 3H), 2.49-2.29 (m, 2H); MS (ES+): 501.20 (M+1); (ES−): 499.10 (M−1).

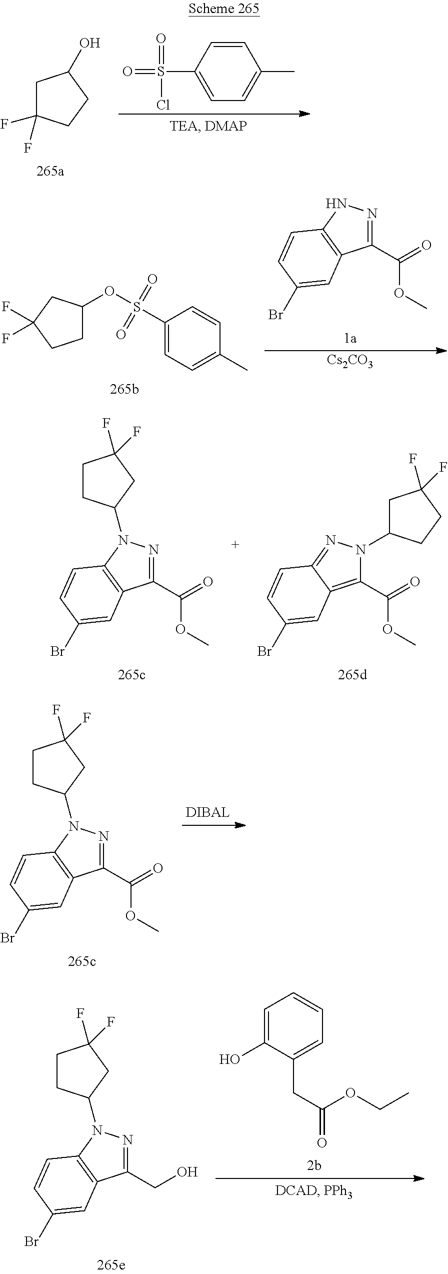

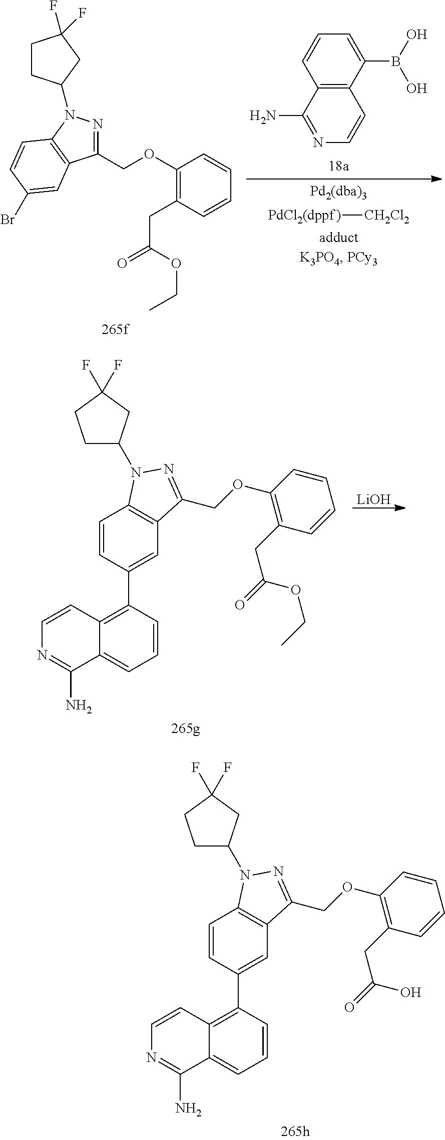

### Preparation of 2-(2-((5-(1-aminoisoquinolin-5-yl)-1-(3,3-difluorocyclopentyl)-1H-indazol-3-yl)methoxy)phenyl)acetic acid (265h)

### Step-1: Preparation of 3,3-difluorocyclopentyl 4-methylbenzenesulfonate (265b)

Compound 265b was prepared according to the procedure reported in step-1 of scheme-86 from 3,3-difluorocyclopentanol (265a) (1 g, 8.19 mmol; CAS #: 883731-65-5) in anhydrous DCM (50 mL) using DMAP (0.050 g, 0.409 mmol), TEA (1.712 mL, 12.28 mmol) and p-toluenesulfonylchloride (3.12 g, 16.38 mmol) to afford after workup and purification using method-AL, 3,3-difluorocyclopentyl 4-methylbenzenesulfonate (265b) (1.5 g, 66.3% yield) as a clear oil; ¹H NMR (300 MHz, DMSO-d₆) δ 7.87-7.75 (m, 2H), 7.57-7.42 (m, 2H), 5.17-5.03 (m, 1H), 3.81-3.61 (m, 4H), 2.43 (s, 3H), 2.16-2.00 (m, 1H), 1.95-1.81 (m, 1H).

### Step-2: Preparation of methyl 5-bromo-1-(3,3-difluorocyclopentyl)-1H-indazole-3-carboxylate (265c) and methyl 5-bromo-2-(3,3-difluorocyclopentyl)-2H-indazole-3-carboxylate (265d)

Compounds 265c and 265d were prepared according to the procedure reported in step-2 of scheme-86 from methyl 5-bromo-1H-indazole-3-carboxylate (1a) (1.385 g, 5.43 mmol) in DMF (20 mL) using Cs₂CO₃ (3.54 g, 10.86 mmol) and 3,3-difluorocyclopentyl 4-methylbenzenesulfonate (265b) (1.5 g, 5.43 mmol) to afford after workup and purification using method-S, methyl 5-bromo-1-(3,3-difluorocyclopentyl)-1H-indazole-3-carboxylate (265c) (650 mg, 33.3% yield) as a white solid; ¹H NMR (300 MHz, DMSO-d₆) δ 8.21 (d, J=1.8 Hz, 1H), 7.91 (d, J=9.0 Hz, 1H), 7.67 (dd. J=9.0, 1.9 Hz, 1H), 5.68-5.51 (m, 1H), 3.94 (s, 3H), 2.99-2.54 (m, 2H), 2.47-2.34 (m, 2H), 2.32-2.12 (m, 2H); MS (ES+): 359.00 & 361.00 (M+1); MS (ES+): 381.00 & 383.00 (M+Na) and methyl 5-bromo-2-(3,3-difluorocyclopentyl)-2H-indazole-3-carboxylate (265d) (420 mg, 21.54% yield) as a white solid; ¹H NMR (300 MHz, DMSO-d₆) δ 8.17 (dd, J=1.9, 0.7 Hz, 1H), 7.83 (dd, J=9.1, 0.8 Hz, 1H), 7.51 (dd. J=9.1, 1.9 Hz, 1H), 6.23-6.04 (m, 1H), 4.00 (s, 3H), 2.95-2.69 (m, 2H), 2.48-2.13 (m, 4H); MS (ES+): 359.10 & 361.10 (M+1).

### Step-3: Preparation of (5-bromo-1-(3,3-difluorocyclopentyl)-1H-indazol-3-yl)methanol (265e)

Compound 265e was prepared according to the procedure reported in step-1 of scheme-2 from methyl 5-bromo-1-(3,3-difluorocyclopentyl)-1H-indazole-3-carboxylate (265c) (650 mg, 1.810 mmol) in DCM (20 mL) using DIBAL (1M solution in DCM, 4.52 mL, 4.52 mmol) to afford after workup and purification using method-F, (5-bromo-1-(3,3-difluorocyclopentyl)-1H-indazol-3-yl)methanol (265e) (580 mg, 97% yield) as a light yellow oil; ¹H NMR (300 MHz, DMSO-d₆) δ 8.07 (d, J=1.8 Hz, 1H), 7.69 (dd, J=8.9, 1.6 Hz, 1H), 7.52 (dt, J=8.9, 1.8 Hz, 1H), 5.46-5.32 (m, 2H), 4.80-4.70 (m, 2H), 2.83-2.56 (m, 2H), 2.44-2.14 (m, 4H); MS (ES+): 331.00 & 333.00 (M+1).

### Step-4: Preparation of ethyl 2-(2-((5-bromo-1-(3,3-difluorocyclopentyl)-1H-indazol-3-yl)methoxy)phenyl)acetate (265f)

Compound 265f was prepared according to the procedure reported in step-2 of scheme-2 from (5-bromo-1-(3,3-difluorocyclopentyl)-1H-indazol-3-yl)methanol (265e) (290 mg, 0.876 mmol) in DCM (10 mL) using ethyl 2-(2-hydroxyphenyl)acetate (2b) (166 mg, 0.920 mmol). PPh₃ (276 mg, 1.051 mmol) and a solution of DCAD (386 mg, 1.051 mmol) in DCM (10 mL) to afford after workup and purification using method-N, ethyl 2-(2-((5-bromo-1-(3,3-difluorocyclopentyl)-1H-indazol-3-yl)methoxy)phenyl)acetate (265f) (340 mg, 79% yield) as a white oil. MS (ES+): 493.10 & 495.10 (M+1).

### Step-5: Preparation of ethyl 2-(2-((5-(1-aminoisoquinolin-5-yl)-1-(3,3-difluorocyclopentyl)-1H-indazol-3-yl)methoxy)phenyl)acetate (265g)

Compound 265g was prepared according to the procedure reported in step-5 of scheme-1 from ethyl 2-(2-((5-bromo-1-(3,3-difluorocyclopentyl)-1H-indazol-3-yl)methoxy)phenyl)acetate (265f) (170 mg, 0.345 mmol) in dioxane/THF (6 mL, 1:1) using (1-aminoisoquinolin-5-yl)boronic acid (18a) (97 mg, 0.517 mmol), K₃PO₄ (2 M aqueous solution, 0.689 mL, 1.378 mmol), PCy₃ (19.33 mg, 0.069 mmol), PdCl₂(dppf)-CH₂Cl₂ adduct (28.1 mg, 0.034 mmol) and Pd₂(dba)₃ (31.6 mg, 0.034 mmol) to afford after workup and purification using method-AM, ethyl 2-(2-((5-(1-aminoisoquinolin-5-yl)-1-(3,3-difluorocyclopentyl)-1H-indazol-3-yl)methoxy)phenyl)acetate (265g) (150 mg, 78% yield) as a clear oil; MS (ES+): 557.25 (M+1).

### Step-6: Preparation of 2-(2-((5-(1-aminoisoquinolin-5-yl)-1-(3,3-difluorocyclopentyl)-1H-indazol-3-yl)methoxy)phenyl)acetic acid (265h)

Compound 265h was prepared according to the procedure reported in step-2 of scheme-1 from ethyl 2-(2-((5-(1-aminoisoquinolin-5-yl)-1-(3,3-difluorocyclopentyl)-1H-indazol-3-yl)methoxy)phenyl)acetate (265g) (150 mg, 0.269 mmol) in MeOH/THF (6 mb, 1:1) using a solution of LiOH·H₂O (67.9 mg, 1.617 mmol) in water (2 mL) to afford after workup and purification using method-G, 2-(2-((5-(1-aminoisoquinolin-5-yl)-1-(3,3-difluorocyclopentyl)-1H-indazol-3-yl)methoxy)phenyl)acetic acid (265h) (45 mg, 31.6% yield) HCl salt as a white solid; ¹H NMR (300 MHz, DMSO-d₆) δ 13.21 (s, 1H, D₂O exchangeable), 12.02 (s, 1H, D₂O exchangeable), 9.13 (s, 2H, D₂O exchangeable), 8.61 (d, J=8.3 Hz, 1H), 7.99-7.80 (m, 4H), 7.62 (d, J=7.2 Hz, 1H), 7.57-7.47 (m, 1H), 7.32-7.21 (m, 2H), 7.21-7.13 (m, 1H), 6.98 (d, J=7.2 Hz, 1H), 6.95-6.82 (m, 1H), 5.63-5.49 (m, 1H), 5.46 (s, 2H), 3.48 (s, 2H), 2.99-2.58 (m, 2H), 2.48-2.14 (m, 4H); 1ºF NMR (282 MHz, DMSO-d₆) δ −87.25-−90.0 (m); MS (ES+): 529.20 (M+1); (ES−): 527.10 (M−1).

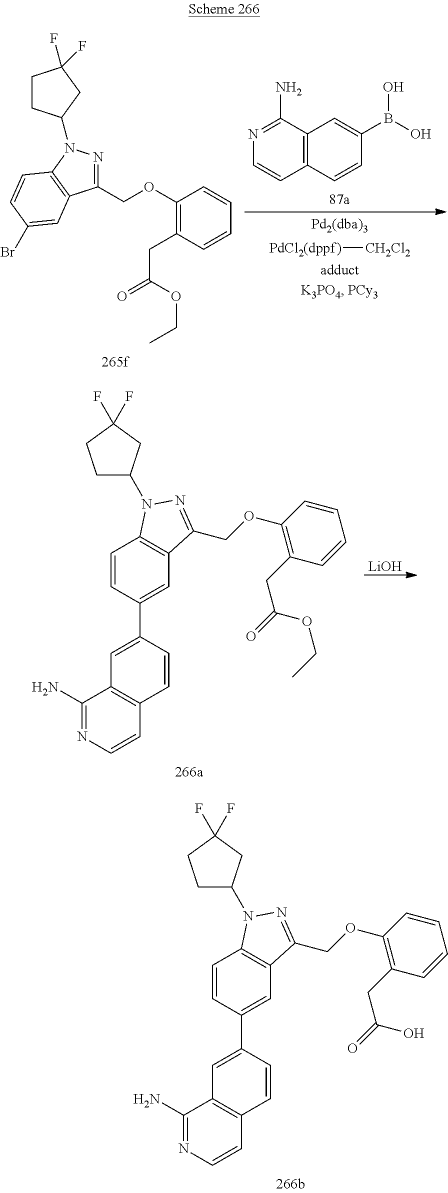

### Preparation of 2-(2-((5-(1-aminoisoquinolin-7-yl)-1-(3,3-difluorocyclopentyl)-1H-indazol-3-yl)methoxy)phenyl)acetic acid (266b)

### Step-1: Preparation of ethyl 2-(2-((5-(1-aminoisoquinolin-7-yl)-1-(3,3-difluorocyclopentyl)-1H-indazol-3-yl)methoxy)phenyl)acetate (266a)

Compound 266a was prepared according to the procedure reported in step-5 of scheme-1 from ethyl 2-(2-((5-bromo-1-(3,3-difluorocyclopentyl)-1H-indazol-3-yl)methoxy)phenyl)acetate (265f) (170 mg, 0.345 mmol) in dioxane/THF (6 mL, 1:1) using 1-aminoisoquinolin-7-ylboronic acid (87a) (97 mg, 0.517 mmol). K₃PO₄ (2 M aqueous solution, 0.689 mL, 1.378 mmol), PCy₃ (19.33 mg, 0.069 mmol), PdCl₂(dppf)-CH₂Cl₂ adduct (28.1 mg, 0.034 mmol) and Pd₂(dba)₃ (31.6 mg, 0.034 mmol) to afford after workup and purification using method-AM, ethyl 2-(2-((5-(1-aminoisoquinolin-7-yl)-1-(3,3-difluorocyclopentyl)-1H-indazol-3-yl)methoxy)phenyl)acetate (266a) (150 mg, 78% yield) as a clear oil; MS (ES+): 557.30 (M+1).

### Step-2: Preparation of 2-(2-((5-(1-aminoisoquinolin-7-yl)-1-(3,3-difluorocyclopentyl)-1H-indazol-3-yl)methoxy)phenyl)acetic acid (266b)

Compound 266b was prepared according to the procedure reported in step-2 of scheme-1 from ethyl 2-(2-((5-(1-aminoisoquinolin-7-yl)-1-(3,3-difluorocyclopentyl)-1H-indazol-3-yl)methoxy)phenyl)acetate (266a) (150 mg, 0.269 mmol) in MeOH/THF (6 mL, 1:1) using a solution of LiOH·H₂O (67.9 mg, 1.617 mmol) in water (2 mL) to afford after workup and purification using method-G, 2-(2-((5-(1-aminoisoquinolin-7-yl)-1-(3,3-difluorocyclopentyl)-1H-indazol-3-yl)methoxy)phenyl)acetic acid (266b) (40 mg, 28.1% yield) HCl salt as a white solid; ¹H NMR (300 MHz, DMSO-d₆) δ 13.13 (s, 1H, D₂O exchangeable), 12.09 (s, 1H, D₂O exchangeable), 9.06 (s, 2H, D₂O exchangeable), 8.94 (s, 1H), 8.46-8.37 (m, 1H), 8.32 (s, 1H), 8.09-8.00 (m, 2H), 8.00-7.90 (m, 1H), 7.69 (d, J=6.9 Hz, 1H), 7.34-7.23 (m, 3H), 7.20 (d, J=7.4 Hz, 1H), 6.97-6.87 (m, 1H), 5.61-5.51 (m, 1H), 5.50 (s, 2H), 3.53 (s, 2H), 2.98-2.59 (m, 2H), 2.48-2.07 (m, 4H); ¹⁹F NMR (282 MHz, DMSO-d₆) δ −87.18-90.0 (m); MS (ES+): 529.20 (M+1); (ES−): 527.10 (M−1).

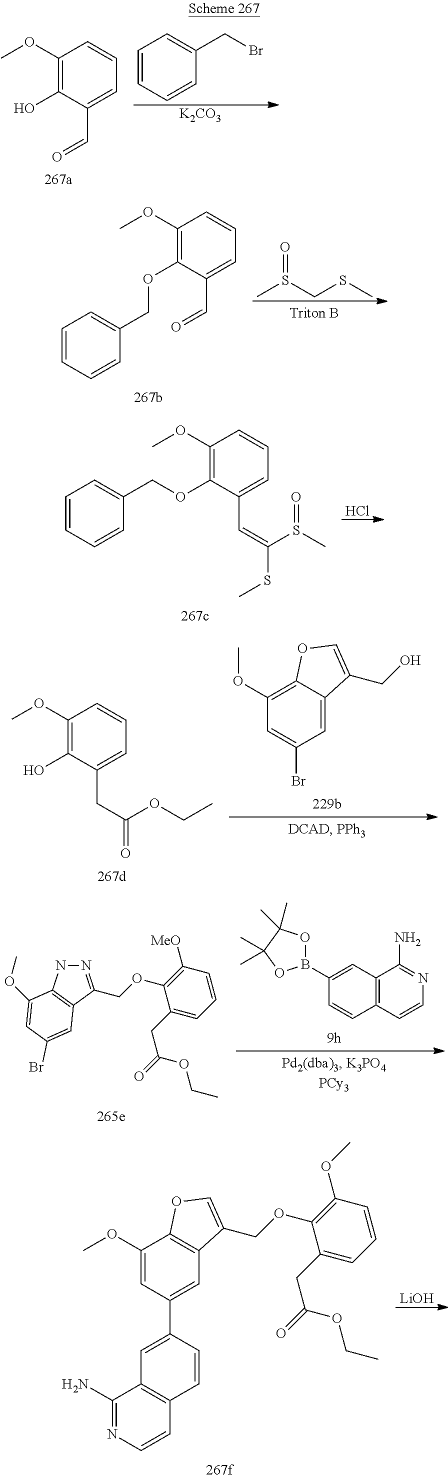

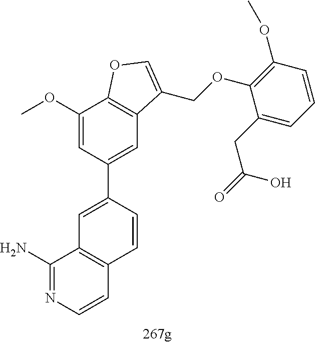 [SMILES: COc1cccc(CC(=O)O)c1OCc1coc2c(OC)cc(-c3ccc4ccnc(N)c4c3)cc12]

### Preparation of 2-(2-((5-(1-aminoisoquinolin-7-yl)-7-methoxybenzofuran-3-yl)methoxy)-3-methoxyphenyl)acetic acid (267g)

### Step-1: Preparation of 2-(benzyloxy)-3-methoxybenzaldehyde (267b)

Compound 267b was prepared according to the procedure reported in step-3 of scheme-106 from 2-hydroxy-3-methoxybenzaldehyde (267a) (5.0 g, 32.86 mmol) in DMF (50 mL) using benzyl bromide (6.74 g, 39.41 mmol), K₂CO₃ (13.62 g, 98.59 mmol) and stirring at RT for 12 h) to afford after workup and purification using method-AY, 2-(benzyloxy)-3-methoxybenzaldehyde (267b) (6.8 g, 85% yield) as an oil; ¹H NMR (300 MHz, DMSO-d₆) δ 10.13 (d, J=0.7 Hz, 1H), 7.55-7.31 (m, 6H), 7.30-7.09 (m, 2H), 5.17 (s, 2H), 3.93 (s, 3H).

### Step-2: Preparation of (Z)-(2-(2-(benzyloxy)-3-methoxyphenyl)-1-(methylsulfinyl)vinyl)(methyl)sulfane (267c)

To a stirred solution of 2-(benzyloxy)-3-methoxybenzaldehyde (267b) (6.8 g, 28.07 mmol) in THF (136 mL) was added methyl((methylsulfinyl)methyl)sulfane (5.58 g, 44.91 mmol) at RT 25% Triton-B in MeOH (9.38 mL, 14.03 mmol) and heated at reflux for 20 h. The reaction mixture was concentrated in vacuum and the residue obtained was dissolved in water and extracted with EtOAc. The organic layers were dried, filtered and concentrated. The residue obtained was purified using method-BJ to afford (E)-(3-(2-(benzyloxy)-3-methoxyphenyl)-1-(methylsulfinyl)allyl)(methyl)sulfane (267c) (8 g, 82% yield) as an oil; ¹H NMR (300 MHz, DMSO-d₆) δ 7.76 (s, 1H), 7.70-7.57 (m, 1H), 7.46-7.36 (m, 2H), 7.41-7.26 (m, 3H), 7.23-7.11 (m, 2H), 4.96 (s, 2H), 3.88 (s, 3H), 2.68 (s, 3H), 2.20 (s, 3H).

### Step-3: Preparation of ethyl 2-(2-hydroxy-3-methoxyphenyl)acetate (267d)

To a stirred solution of (E)-(3-(2-(benzyloxy)-3-methoxyphenyl)-1-(methylsulfinyl)allyl)(methyl)sulfane (267c) (8.0 g, 22.96 mmol) in ethanol (40 mL) was added at RT HCl in EtOH (40 mL) and heated at reflux for 2 h. The reaction mixture was concentrated, diluted with water and extracted with EtOAc. The combined organic layers were dried, filtered and concentrated. The residue obtained was purified using method-AY to give ethyl 2-(2-hydroxy-3-methoxyphenyl)acetate (267d) (2.6 g, 54% yield) as an oil; ¹H NMR (300 MHz, DMSO-d₆) δ 8.67 (s, 1H), 6.85 (dt, J=7.8, 3.9 Hz, 1H), 6.71 (q, J=3.3, 2.6 Hz, 2H), 4.05 (q, J=7.0 Hz, 2H), 3.78 (s, 3H), 3.54 (s, 2H), 1.17 (t, J=7.1 Hz, 3H).

### Step-4: Preparation of ethyl 2-(2-((5-bromo-7-methoxybenzofuran-3-yl)methoxy)-3-methoxyphenyl)acetate (267e)

Compound 267e was prepared according to the procedure reported in step-2 of scheme-2 from (5-bromo-7-methoxybenzofuran-3-yl)methanol (229b) (350 mg, 1.361 mmol) in DCM (10) mL) using PPh₃ (411 mg, 1.566 mmol), ethyl 2-(2-hydroxy-3-methoxyphenyl)acetate (267d) (315 mg, 1.498 mmol) in DCM (10 mL) and a solution of DCAD (575 mg, 1.566 mmol) in DCM (5 mL) to afford after workup and purification using method-BD, ethyl 2-(2-((5-bromo-7-methoxybenzofuran-3-yl)methoxy)-3-methoxyphenyl)acetate (267e) (370 mg, 60.5% yield) as a white solid; MS (ES+): 449.1 (M+1).

### Step-5: Preparation of ethyl 2-(2-((5-(1-aminoisoquinolin-7-yl)-7-methoxybenzofuran-3-yl)methoxy)-3-methoxyphenyl)acetate (267f)

Compound 267f was prepared according to the procedure reported in step-3 of scheme-112 from ethyl 2-(2-((5-bromo-7-methoxybenzofuran-3-yl)methoxy)-3-methoxyphenyl)acetate (267e) (150 mg, 0.334 mmol) in dioxane (12 mL) using 7-(4,4,5,5-tetramethyl-1,3,2-dioxaborolan-2-yl)isoquinolin-1-amine (9h) (135 mg, 0.501 mmol). K₃PO₄ (4M aqueous solution, 0.334 mL, 1.335 mmol), PCy₃ (28.1 mg, 0.100 mmol) and Pd₂(dba)₃ (45.9 mg, 0.050 mmol) to afford after workup and purification using method-E, ethyl 2-(2-((5-(1-aminoisoquinolin-7-yl)-7-methoxybenzofuran-3-yl)methoxy)-3-methoxyphenyl)acetate (267f) (103 mg, 0.201 mmol, 60.2% yield) as a clear oil; MS (ES+): 513.2 (M+1).

### Step-6: Preparation of 2-(2-((5-(1-aminoisoquinolin-7-yl)-7-methoxybenzofuran-3-yl)methoxy)-3-methoxyphenyl)acetic acid (267g)

Compound 267g was prepared according to the procedure reported in step-2 of scheme-1 from ethyl 2-(2-((5-(1-aminoisoquinolin-7-yl)-7-methoxy benzofuran-3-yl)methoxy)-3-methoxyphenyl)acetate (267f) (94 mg, 0.183 mmol) in THF (1.2 mL). ACN (0.6 mL) using a 1N solution of LiOH·H₂O (0.55 mL, 0.55 mmol) to afford after workup and purification using method-G, 2-(2-((5-(1-aminoisoquinolin-7-yl)-7-methoxybenzofuran-3-yl)methoxy)-3-methoxyphenyl)acetic acid (267g) (43 mg, 48% yield) HCl salt as an off white solid; ¹H NMR (300 MHz, DMSO-d₆) δ 13.17 (s, 1H, D₂O exchangeable), 12.22 (s, 1H, D₂O exchangeable), 9.21 (s, 1H, D₂O exchangeable), 8.95 (s, 1H), 8.38 (dd, J=8.5, 1.6 Hz, 1H), 8.08 (t, J=4.3 Hz, 2H), 7.78 (d, J=1.5 Hz, 1H), 7.71 (d, J=6.9 Hz, 1H), 7.46 (d, J=1.6 Hz, 1H), 7.30 (d, J=7.0 Hz, 1H), 7.09-7.00 (m, 2H), 6.82 (dd, J=5.6, 3.6 Hz, 1H), 5.17 (s, 2H), 4.12 (s, 3H), 3.88 (s, 3H), 3.51 (s, 2H); MS (ES+): 485.2 (M+1); (ES−): 483.1 (M−1).

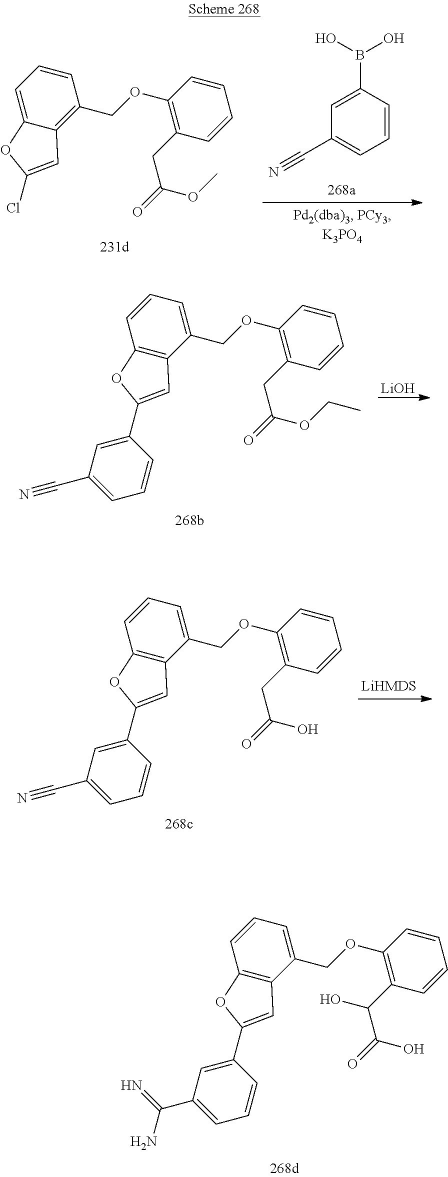

### Preparation of 2-(2-((2-(3-carbamimidoylphenyl)benzofuran-4-yl)methoxy)phenyl)-2-hydroxyacetic acid (268d)

### Step-1: Preparation of ethyl 2-(2-((2-(3-cyanophenyl)benzofuran-4-yl)methoxy)phenyl)acetate (268b)

Compound 268b was prepared according to the procedure reported in step-3 of scheme-112 from ethyl 2-(2-((2-chlorobenzofuran-4-yl)methoxy)phenyl)acetate (231d) (200 mg, 0.580 mmol) in Dioxane/2Me-THF (12 mL, 1:2) using 3-cyanophenylboronic acid (268a) (170 mg, 1.160 mmol; CAS #150255-96-2), K₃PO₄ (4M aqueous solution, 0.580 mL, 2.32 mmol), Pd₂(dba)₃ (106 mg, 0.116 mmol) and PCy₃ (65.1 mg, 0.232 mmol) to afford after workup and purification using method-C, ethyl 2-(2-((2-(3-cyanophenyl)benzofuran-4-yl)methoxy)phenyl)acetate (268b) (149 mg, 62.4% yield) as a yellow solid; ¹H NMR (300 MHz, DMSO-d₆) δ 8.41 (t, J=1.6 Hz, 1H), 8.25 (dt, J=8.1, 1.4 Hz, 1H), 7.88 (dt, J=7.7, 1.4 Hz, 1H), 7.81 (d, J=1.0 Hz, 1H), 7.74 (t, J=7.8 Hz, 1H), 7.66 (dd, J=5.7, 3.6 Hz, 1H), 7.42-7.36 (m, 2H), 7.33-7.22 (m, 2H), 7.16 (d, J=8.1 Hz, 1H), 6.99-6.88 (m, 1H), 5.38 (s, 2H), 3.90 (q, J=7.1 Hz, 2H), 3.64 (s, 2H), 0.97 (t, J=7.1 Hz, 3H).

### Step-2: Preparation of 2-(2-((2-(3-cyanophenyl)benzofuran-4-yl)methoxy)phenyl)acetic acid (268c)

Compound 268c was prepared according to the procedure reported in step-2 of scheme-1 from ethyl 2-(2-((2-(3-cyanophenyl)benzofuran-4-yl)methoxy)phenyl)acetate (268b) (146 mg, 0.355 mmol) in MeOH/THF (6 mL, 1:1) using a solution of LiOH·H₂O (89 mg, 2.129 mmol) in water (2 mL) to afford after workup and purification using method-BG, 2-(2-((2-(3-cyanophenyl)benzofuran-4-yl)methoxy)phenyl)acetic acid (268c) as a white solid; MS (ES−): 382.1 (M−1).

### Step-3: Preparation of 2-(2-((2-(3-carbamimidoylphenyl)benzofuran-4-yl)methoxy)phenyl)-2-hydroxyacetic acid (268d)

To a solution of 2-(2-((2-(3-cyanophenyl)benzofuran-4-yl)methoxy)phenyl)acetic acid (268c) (132 mg, 0.344 mmol) in diethyl ether (20 mL) was added LiHMDS (1 M in THF) (4.75 mL, 4.75 mmol) and stirred at RT overnight. The reaction mixture was quenched with 2 M HCl (4 ml) concentrated in vacuum and purified using method-G, to afford 2-(2-((2-(3-carbamimidoylphenyl)benzofuran-4-yl)methoxy)phenyl)-2-hydroxyacetic acid (268d) (28 mg, 19.53% yield) HCl salt as a white solid. ¹H NMR (300 MHz, DMSO-d₆) δ 12.52 (s, 1H, D₂O exchangeable), 9.55 (s, 2H, D₂O exchangeable), 9.30 (s, 2H, D₂O exchangeable), 8.44-8.32 (m, 1H), 8.32-8.17 (m, 1H), 7.91-7.81 (m, 2H), 7.75 (t, J=7.8 Hz, 1H), 7.66 (d, J=7.9 Hz, 1H), 7.51-7.35 (m, 3H), 7.29 (td, J=7.7, 1.8 Hz, 1H), 7.15 (d, J=8.2 Hz, 1H), 6.98 (t, J=7.4 Hz, 1H), 5.76 (s, 1H, D₂O exchangeable), 5.54-5.32 (m, 3H); ¹H NMR (300) MHz, DMSO-de, D₂O exchange) δ 8.28 (d, J=1.8 Hz, 1H), 8.23 (dt, J=7.3, 1.7 Hz, 1H), 7.82-7.67 (m, 3H), 7.62 (d, J=7.6 Hz, 1H), 7.45-7.23 (m, 4H), 7.12 (d, J=8.3 Hz, 1H), 6.96 (t, J=7.4 Hz, 1H), 5.45-5.33 (m, 3H); MS (ES+): 417.1 (M+1); (ES−): 415.1 (M−1).

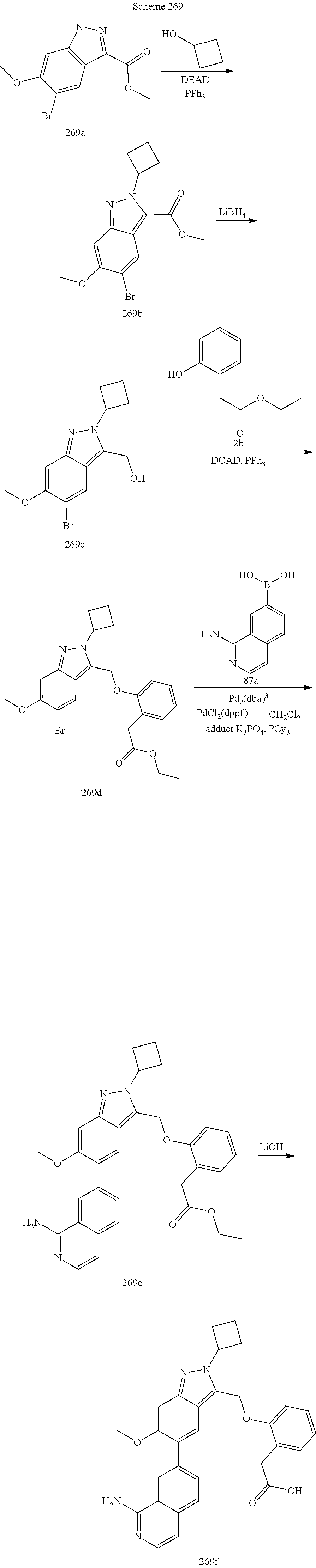

### Preparation of 2-(2-((5-(1-aminoisoquinolin-7-yl)-2-cyclobutyl-6-methoxy-2H-indazol-3-yl)methoxy)phenyl)acetic acid (269f)

### Step-1: Preparation of methyl 5-bromo-2-cyclobutyl-6-methoxy-2H-indazole-3-carboxylate (269b)

Compound 269b was prepared according to the procedure reported in step-1 of scheme-8 from methyl 5-bromo-6-methoxy-1H-indazole-3-carboxylate (269a) (2 g, 7.02 mmol; CAS #1134328-15-6) in THF using PPh₃ (3.68 g, 14.03 mmol), cyclobutanol (1.098 mL, 14.03 mmol) and a solution of DEAD (2.201 mL, 14.03 mmol) in THF to afford after workup and purification using method-N, methyl 5-bromo-2-cyclobutyl-6-methoxy-2H-indazole-3-carboxylate (269b) (1.64 g, 68.9% yield) as yellow solid; MS (ES+) 339.0 (M+1).

### Step-2: Preparation of (5-bromo-2-cyclobutyl-6-methoxy-2H-indazol-3-yl)methanol (269c)

Compound 269c was prepared according to the procedure reported in step-2 of scheme-119 from methyl 5-bromo-2-cyclobutyl-6-methoxy-2H-indazole-3-carboxylate (269b) (1.64 g, 4.84 mmol) in THF (100 mL) using LiBH₄ (6.04 mL, 12.09 mmol) and MeOH (0.489 mL, 12.09 mmol) to afford after workup and purification using method-AW. (5-bromo-2-cyclobutyl-6-methoxy-2H-indazol-3-yl)methanol (269c) (1.15 g, 3.70 mmol, 76% yield) as a clear oil; MS (ES+) 311.0 (M+1).

### Step-3: Preparation of ethyl 2-(2-((5-bromo-2-cyclobutyl-6-methoxy-2H-indazol-3-yl)methoxy)phenyl)acetate (269d)

Compound 269d was prepared according to the procedure reported in step-2 of scheme-2 from (5-bromo-2-cyclobutyl-6-methoxy-2H-indazol-3-yl)methanol (269c) (240 mg, 0.771 mmol) in DCM (20 mL) using PPh₃ (243 mg, 0.926 mmol) ethyl 2-(2-hydroxyphenyl)acetate (2b) (167 mg, 0.926 mmol) and a solution of DCAD (0.182 mL, 0.926 mmol) in DCM (10 mL) to afford after workup and purification using method-P, ethyl 2-(2-((5-bromo-2-cyclobutyl-6-methoxy-2H-indazol-3-yl)methoxy)phenyl)acetate (269d) (750 mg) as a clear oil; MS (ES+): 473.1 (M+1).

### Step-4: Preparation of ethyl 2-(2-((5-(1-aminoisoquinolin-7-yl)-2-cyclobutyl-6-methoxy-2H-indazol-3-yl)methoxy)phenyl)acetate (269e)

Compound 269e was prepared according to the procedure reported in step-5 of scheme-1 from ethyl 2-(2-((5-bromo-2-cyclobutyl-6-methoxy-2H-indazol-3-yl)methoxy)phenyl)acetate (269d) (200 mg, 0.423 mmol) in dioxane/THF (8 mL, 1:1) using 1-aminoisoquinolin-7-ylboronic acid (87a) (103 mg, 0.549 mmol), K₃PO₄ (2 M aqueous solution, 0.423 mL, 1.690 mmol), PCy₃ (23.70 mg, 0.085 mmol), Pd₂(dba)₃ (38.7 mg, 0.042 mmol) and PdCl₂(dppf)-CH₂Cl₂ adduct (34.5 mg, 0.042 mmol) to afford after workup and purification using method-AE, ethyl 2-(2-((5-(1-aminoisoquinolin-7-yl)-2-cyclobutyl-6-methoxy-2H-indazol-3-yl)methoxy)phenyl)acetate (269e) (65 mg, 28.7% yield) as a yellow solid; MS (ES+): $37.2 (M+1).

### Step-5: Preparation of 2-(2-((5-(1-aminoisoquinolin-7-yl)-2-cyclobutyl-6-methoxy-2H-indazol-3-yl)methoxy)phenyl)acetic acid (269f)

Compound 269f was prepared according to the procedure reported step-2 of scheme-1 from ethyl 2-(2-((5-(1-aminoisoquinolin-7-yl)-2-cyclobutyl-6-methoxy-2H-indazol-3-yl)methoxy)phenyl)acetate (269e) (37 mg, 0.069 mmol) in THF (3 mL) using a solution of LiOH·H₂O (8.68 mg, 0.207 mmol) in water (1 mL) to afford after workup and purification using method-M, 2-(2-((5-(1-aminoisoquinolin-7-yl)-2-cyclobutyl-6-methoxy-2H-indazol-3-yl)methoxy)phenyl)acetic acid (269f) HCl salt as a white solid; ¹H NMR (300 MHz, DMSO-d₆) δ 13.29-12.96 (m, 1H, D₂O exchangeable), 9.27-8.84 (m, 2H, D₂O exchangeable), 8.68 (s, 1H), 8.15-8.03 (m, 1H), 8.02-7.93 (m, 1H), 7.93-7.86 (m, 1H), 7.75-7.61 (m, 1H), 7.40-7.12 (m, 5H), 7.04-6.87 (m, 1H), 5.53 (s, 2H), 5.24 (p. J=8.3, 7.7 Hz, 1H), 3.84 (s, 3H), 3.49 (s, 2H), 2.85-2.54 (m, 4H), 2.04-1.74 (m, 2H); MS (ES+) 509.2 (M+1).

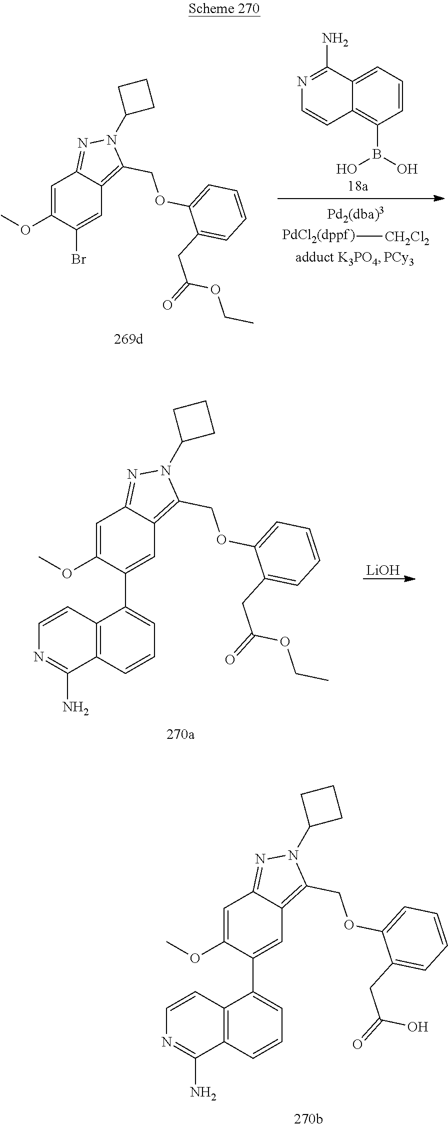

### Preparation of 2-(2-((5-(1-aminoisoquinolin-5-yl)-2-cyclobutyl-6-methoxy-2H-indazol-3-yl)methoxy)phenyl)acetic acid (270b)

### Step-1: Preparation of ethyl 2-(2-((5-(1-aminoisoquinolin-5-yl)-2-cyclobutyl-6-methoxy-2H-indazol-3-yl)methoxy)phenyl)acetate (270a)

Compound 270a was prepared according to the procedure reported in step-5 of scheme-1 from ethyl 2-(2-((5-bromo-2-cyclobutyl-6-methoxy-2H-indazol-3-yl)methoxy)phenyl)acetate (269d) (145 mg, 0.306 mmol) in dioxane/THF (8 mL, 1:1) using (1-aminoisoquinolin-5-yl)boronic acid (18a) (74.9 mg, 0.398 mmol), K₃PO₄ (2 M aqueous solution, 0.306 mL, 1.225 mmol). PCy₃ (17.18 mg, 0.061 mmol). Pd₂(dba)₃ (28.1 mg, 0.031 mmol) and PdCl₂(dppf)-CH₂Cl₂ adduct (25.02 mg, 0.031 mmol) to afford after workup and purification using method-AE, ethyl 2-(2-((5-(1-aminoisoquinolin-5-yl)-2-cyclobutyl-6-methoxy-2H-indazol-3-yl)methoxy)phenyl)acetate (270a) (30 mg, 18.25% yield) as a yellow solid; MS (ES+): 537.3 (M+1).

### Step-2: Preparation of 2-(2-((5-(1-aminoisoquinolin-5-yl)-2-cyclobutyl-6-methoxy-2H-indazol-3-yl)methoxy)phenyl)acetic acid (270b)

Compound 270b was prepared according to the procedure reported in step-5 of scheme-1 from ethyl 2-(2-((5-(1-aminoisoquinolin-5-yl)-2-cyclobutyl-6-methoxy-2H-indazol-3-yl)methoxy)phenyl)acetate (270a) (65 mg, 0.121 mmol) in THF (3 mL) using a solution of LiOH·H₂O (15.25 mg, 0.363 mmol) in water (1 mL) to afford after workup and purification using method-M, 2-(2-((5-(1-aminoisoquinolin-5-yl)-2-cyclobutyl-6-methoxy-2H-indazol-3-yl)methoxy)phenyl)acetic acid (270b) (4.0 mg, 7.87 μmol, 6.49% yield) HCl salt as a white solid; ¹H NMR (300 MHz, DMSO-d₆) δ 13.21 (s, 1H, D₂O exchangeable), 9.15 (s, 2H, D₂O exchangeable), 8.60 (d, J=7.6 Hz, 1H), 7.91-7.78 (m, 2H), 7.75 (s, 1H), 7.63-7.52 (m, 1H), 7.30-7.13 (m, 4H), 7.00-6.85 (m, 1H), 6.63 (d, J=7.2 Hz, 1H), 5.51 (s, 2H), 5.35-5.16 (m, 1H), 3.70 (s, 3H), 3.48 (s, 2H), 2.88-2.52 (m, 4H), 2.02-1.79 (m, 2H); MS (ES+): 509.2 (M+1).

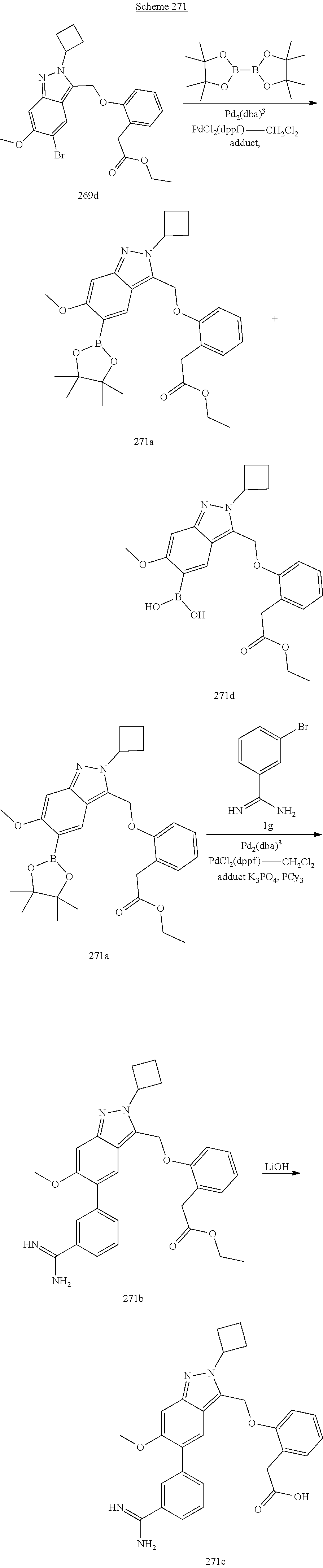

### Preparation of 2-(2-((5-(3-carbamimidoylphenyl)-2-cyclobutyl-6-methoxy-2H-indazol-3-yl)methoxy)phenyl)acetic acid (271c)

### Step-1: Preparation of ethyl 2-(2-((2-cyclobutyl-6-methoxy-5-(4,4,5,5-tetramethyl-1,3,2-dioxaborolan-2-yl)-2H-indazol-3-yl)methoxy)phenyl)acetate (271a)

Compound 271a was prepared according to the procedure reported in step-4 of scheme-1 from ethyl 2-(2-((5-bromo-2-cyclobutyl-6-methoxy-2H-indazol-3-yl)methoxy)phenyl)acetate (269d) (45 mg, 0.095 mmol) in anhydrous dioxane (3 mL) using BISPIN (48.3 mg, 0.190 mmol), KOAc (23.32 mg, 0.238 mmol) and PdCl₂(dppf)-CH₂Cl₂ adduct (4.66 mg, 5.70 μmol) to afford after workup and purification using method-Z, a mixture of ethyl 2-(2-((2-cyclobutyl-6-methoxy-5-(4,4,5,5-tetramethyl-1,3,2-dioxaborolan-2-yl)-2H-indazol-3-yl)methoxy)phenyl)acetate (271a) (40 mg, 81% yield) and 2-cyclobutyl-3-((2-(2-ethoxy-2-oxoethyl)phenoxy)methyl)-6-methoxy-2H-indazol-5-ylboronic acid (271d); MS (ES+) 521.3 (M+1).

### Step-2: Preparation of ethyl 2-(2-((5-(3-carbamimidoylphenyl)-2-cyclobutyl-6-methoxy-2H-indazol-3-yl)methoxy)phenyl)acetate (271b)

Compound 271b was prepared according to the procedure reported in step-5 of scheme-1 from ethyl 2-(2-((2-cyclobutyl-6-methoxy-5-(4,4,5,5-tetramethyl-1,3,2-dioxaborolan-2-yl)-2H-indazol-3-yl)methoxy)phenyl)acetate (271a) (40 mg, 0.077 mmol) in dioxane/THF (8 mL, 1:1) using 3-bromobenzimidamide (1g) (15.3 mg, 0.077 mmol), K₃PO₄ (2 M aqueous solution, 0.077 mL, 0.307 mmol), PCy₃ (4.31 mg, 0.015 mmol), Pd₂(dba)₃ (7.04 mg, 7.69 μmol) and PdCl₂(dppf)-CH₂Cl₂ adduct (6.28 mg, 7.69 μmol) to afford after workup and purification using method-BK, ethyl 2-(2-((5-(3-carbamimidoylphenyl)-2-cyclobutyl-6-methoxy-2H-indazol-3-yl)methoxy)phenyl)acetate (271b) (37 mg, 94% yield) as a yellow solid; MS (ES+): 513.2 (M+1).

### Step-3: Preparation of 2-(2-((5-(3-carbamimidoylphenyl)-2-cyclobutyl-6-methoxy-2H-indazol-3-yl)methoxy)phenyl)acetic acid (271c)

Compound 271e was prepared according to the procedure reported in step-5 of scheme-1 from ethyl 2-(2-((5-(3-carbamimidoylphenyl)-2-cyclobutyl-6-methoxy-2H-indazol-3-yl)methoxy)phenyl)acetate (271b) (30 mg, 0.059 mmol) in THF (3 mL) using a solution of LiOH·H₂O (7.37 mg, 0.176 mmol) in water (1 mL) in water (1 mL) to afford after workup and purification using method-M, 2-(2-((5-(3-carbamimidoylphenyl)-2-cyclobutyl-6-methoxy-2H-indazol-3-yl)methoxy)phenyl)acetic acid (271c) (4.6 mg, 16.22% yield) HCl salt as a white solid; ¹H NMR (300 MHz, DMSO-d₆) δ 9.37 (s, 2H, D₂O exchangeable), 9.11-8.90 (m, 2H, D₂O) exchangeable), 7.96-7.82 (m, 3H), 7.82-7.71 (m, 1H), 7.71-7.59 (m, 1H), 7.37-7.25 (m, 2H), 7.25-7.17 (m, 2H), 7.02-6.89 (m, 1H), 5.52 (s, 2H), 5.33-5.16 (m, 1H), 3.84 (s, 3H), 3.48 (s, 2H), 2.84-2.56 (m, 4H), 2.00-1.79 (m, 2H); MS (ES+): 485.2 (M+1).

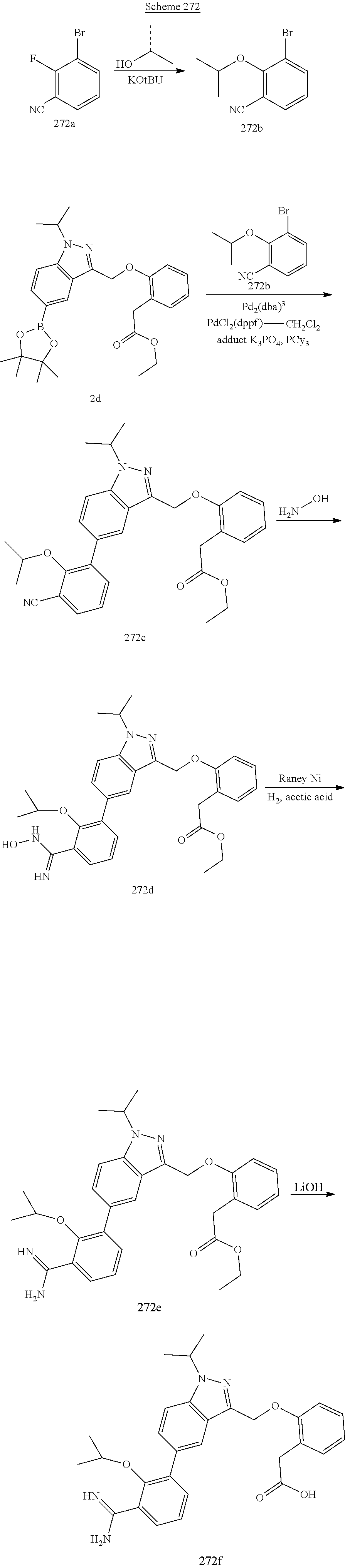

### Preparation of 2-(2-((5-(3-carbamimidoyl-2-isopropoxyphenyl)-1-isopropyl-1H-indazol-3-yl)methoxy)phenyl)acetic acid (272f)

### Step-1: Preparation of 3-bromo-2-isopropoxybenzonitrile (272b)

To a solution of propan-2-ol (3005 mg, 50 mmol) in anhydrous THF (20 mL) at 0° C. was added a solution of potassium 2-methylpropan-2-olate (617 mg, 5.50 mmol) in anhydrous THF (20 mL) under a nitrogen atmosphere and stirred for 10 min at RT. To this mixture was added 3-bromo-2-fluorobenzonitrile (272a) (1000 mg, 5.00 mmol; CAS #840481-82-5) and stirred at RT for 15 h. Solvent was partially removed, and ether was added to this mixture. The organic layer was washed with saturated NaHCO₃, brine, dried, filtered and concentrated in vacuum. The residue obtained was purified using method-AC to give 3-bromo-2-isopropoxybenzonitrile (272b) (800 mg, 66.6% yield) as a white solid; ¹H NMR (300 MHz, DMSO-d₆) δ 8.00 (dd, J=8.1, 1.6 Hz, 1H), 7.84 (dd, J=7.8, 1.6 Hz, 1H), 7.24 (t, J=7.9 Hz, 1H), 4.66 (hept, J=6.1 Hz, 1H), 1.34 (d, J=6.1 Hz, 6H); MS (ES+): 240.00 & 242.00 (M+1).

### Step-2: Preparation of ethyl 2-(2-((5-(3-cyano-2-isopropoxyphenyl)-1-isopropyl-1H-indazol-3-yl)methoxy)phenyl)acetate (272c)

Compound 272c was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (6 mL, 1:1) using 3-bromo-2-isopropoxybenzonitrile (272b) (326 mg, 1.359 mmol). K₃PO₄ (2 M aqueous solution, 2.090 mL, 4.18 mmol), PCy₃ (58.6 mg, 0.209 mmol), PdCl₂(dppf)-CH₂Cl₂ adduct (85 mg, 0.105 mmol) and Pd₂(dba)₃ (96 mg, 0.105 mmol) to afford after workup and purification using method-P, ethyl 2-(2-((5-(3-cyano-2-isopropoxyphenyl)-1-isopropyl-1H-indazol-3-yl)methoxy)phenyl)acetate (272c) (340 mg, 63.6% yield) as a clear oil; ¹H NMR (300 MHz, DMSO-d₆) δ 7.95-7.91 (m, 1H), 7.85-7.72 (m, 3H), 7.62 (dd, J=8.8, 1.6 Hz, 1H), 7.37 (t, J=7.7 Hz, 1H), 7.30-7.24 (m, 2H), 7.18 (d, J=7.3 Hz, 1H), 6.95-6.87 (m, 1H), 5.42 (s, 2H), 5.05 (p, J=6.6 Hz, 1H), 3.88 (p, J=6.0 Hz, 1H), 3.68 (q, J=7.1 Hz, 2H), 3.52 (s, 2H), 1.52 (d, J=6.6 Hz, 6H), 0.99 (d, J=6.1 Hz, 6H), 0.81 (t, J=7.1 Hz, 3H); MS (ES+): 512.20 (M+1); 534.20 (M+Na).

### Step-3: Preparation of ethyl 2-(2-((5-(3-(N-hydroxycarbamimidoyl)-2-isopropoxyphenyl)-1-isopropyl-1H-indazol-3-yl)methoxy)phenyl)acetate (272d)

Compound 272d was prepared according to the procedure reported in step-5 of scheme-23 from ethyl 2-(2-((5-(3-cyano-2-isopropoxyphenyl)-1-isopropyl-1H-indazol-3-yl)methoxy)phenyl)acetate (272c) (340 mg, 0.665 mmol) in EtOH (10 mL) using hydroxylamine (0.439 mL, 6.65 mmol) to afford after workup and purification using method-E, ethyl 2-(2-((5-(3-(N-hydroxycarbamimidoyl)-2-isopropoxyphenyl)-1-isopropyl-1H-indazol-3-yl)methoxy)phenyl)acetate (272d) (200 mg, 55.3% yield) as a white solid; MS (ES+): 545.30 (M+1).

### Step-4: Preparation of ethyl 2-(2-((5-(3-carbamimidoyl-2-isopropoxyphenyl)-1-isopropyl-1H-indazol-3-yl)methoxy)phenyl)acetate (272e)

Compound 272e was prepared according to the procedure reported in step-1 of scheme-24 from ethyl 2-(2-((5-(3-(N-hydroxycarbamimidoyl)-2-isopropoxyphenyl)-1-isopropyl-1H-indazol-3-yl)methoxy)phenyl)acetate (272d) (200 mg, 0.367 mmol) in EtOH (10 mL) using AcOH (0.021 mL, 0.367 mmol), Raney Nickel (0.367 mmol) and hydrogen (1 atm) to afford after workup and purification using method-AI, ethyl 2-(2-((5-(3-carbamimidoyl-2-isopropoxyphenyl)-1-isopropyl-1H-indazol-3-yl)methoxy)phenyl)acetate (272e) (150 mg, 77% yield) as a colorless oil; MS (ES+): 529.30 (M+1).

### Step-5: Preparation of 2-(2-((5-(3-carbamimidoyl-2-isopropoxyphenyl)-1-isopropyl-1H-indazol-3-yl)methoxy)phenyl)acetic acid (272f)

Compound 272f was prepared according to the procedure reported step-2 of scheme-1 from ethyl 2-(2-((5-(3-carbamimidoyl-2-isopropoxyphenyl)-1-isopropyl-1H-indazol-3-yl)methoxy)phenyl)acetate (272e) (150 mg, 0.284 mmol) in MeOH/THF (6 mL, 1:1) using a solution of LiOH·H₂O (71.4 mg, 1.702 mmol) in water (2 mL) to afford after workup and purification using method-G, 2-(2-((5-(3-carbamimidoyl-2-isopropoxyphenyl)-1-isopropyl-1H-indazol-3-yl)methoxy)phenyl)acetic acid (272f) (105 mg, 73.9% yield) HCl salt as a white solid; ¹H NMR (300 MHz, DMSO-d₆) δ 12.10 (s, 1H, D₂O exchangeable), 9.28 (s, 2H, D₂O exchangeable), 9.18 (s, 2H, D₂O exchangeable), 7.97 (d, J=1.5 Hz, 1H), 7.83 (d, J=8.8 Hz, 1H), 7.73-7.63 (m, 2H), 7.52 (dd, J=7.7, 1.8 Hz, 1H), 7.38 (t, J=7.7 Hz, 1H), 7.30-7.22 (m, 2H), 7.22-7.15 (m, 1H), 6.97-6.84 (m, 1H), 5.43 (s, 2H), 5.14-4.94 (m, 1H), 3.77-3.62 (m, 1H), 3.49 (s, 2H), 1.52 (d, J=6.5 Hz, 6H), 0.89 (d, J=6.1 Hz, 6H); MS (ES+): 501.20 (M+1); MS (ES−): 499.20 (M−1); Analysis calculated for C₂₉H₃₂N₄O₄·HCl·1.5H₂O: C, 61.75; H, 6.43; Cl, 6.28; N, 9.93; Found: C, 61.61; H, 6.47; Cl, 6.30; N, 9.96.

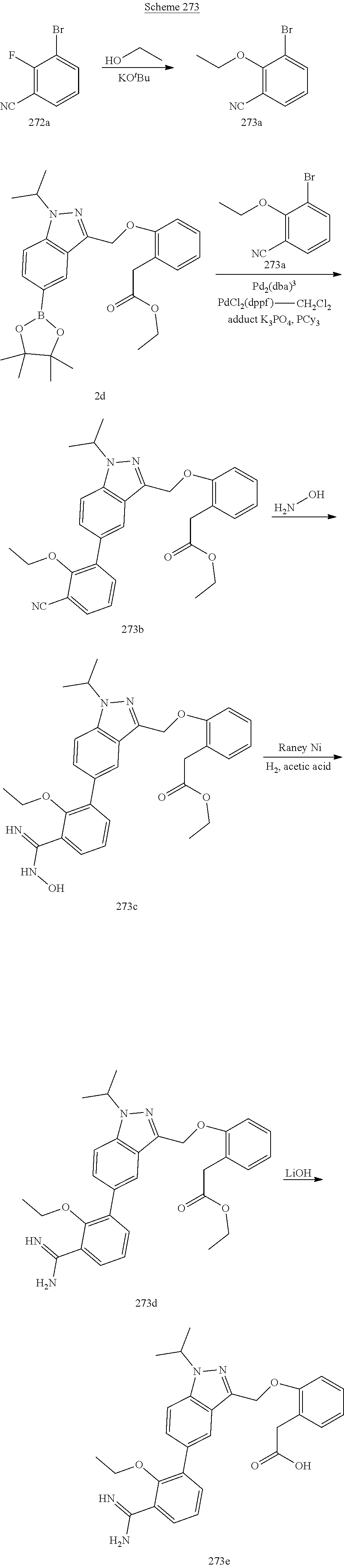

### Preparation of 2-(2-((5-(3-carbamimidoyl-2-ethoxyphenyl)-1-isopropyl-1H-indazol-3-yl)methoxy)phenyl)acetic acid (273e)

### Step-1: Preparation of 3-bromo-2-ethoxybenzonitrile (273a)

Compound 273a was prepared according to the procedure reported in step-1 of scheme-272 from ethanol (2303 mg, 50.0 mmol) in anhydrous THF (20 mL) using a solution of potassium 2-methylpropan-2-olate (617 mg, 5.50 mmol) in anhydrous THF (20 mL) and 3-bromo-2-fluorobenzonitrile (272a) (1000 mg, 5.00 mmol) to afford after workup and purification using method-P, 3-bromo-2-ethoxybenzonitrile (273a) (1 g, 88% yield) as a white solid. ¹H NMR (300 MHz, DMSO-d₆) δ 8.00 (dd, J=8.1, 1.5 Hz, 1H), 7.85 (dd, J=7.8, 1.6 Hz, 1H), 7.25 (t, J=7.9 Hz, 1H), 4.20 (q, J=7.0 Hz, 2H), 1.40 (t, J=7.0 Hz, 3H).

### Step-2: Preparation of ethyl 2-(2-((5-(3-cyano-2-ethoxyphenyl)-1-isopropyl-1H-indazol-3-yl)methoxy)phenyl)acetate (273b)

Compound 273b was prepared according to the procedure reported in step-5 of scheme-1 from ethyl 2-(2-((1-isopropyl-5-(4,4,5,5-tetramethyl-1,3,2-dioxaborolan-2-yl)-1H-indazol-3-yl)methoxy)phenyl)acetate (2d) (500 mg, 1.045 mmol) in dioxane/THF (6 mL, 1:1) using 3-bromo-2-ethoxybenzonitrile (273a) (307 mg, 1.359 mmol), K₃PO₄ (2 M aqueous solution, 2.090 mL, 4.18 mmol), PCy₃ (58.6 mg, 0.209 mmol). PdCl₂(dppf)-CH₂Cl₂ adduct (85 mg, 0.105 mmol) and Pd₂(dba)₃ (96 mg, 0.105 mmol) to afford after workup and purification using method-P, ethyl 2-(2-((5-(3-cyano-2-ethoxyphenyl)-1-isopropyl-1H-indazol-3-yl)methoxy)phenyl)acetate (273b) (430 mg, 83% yield) as a clear oil; ¹H NMR (300 MHZ, DMSO-d₆) δ 7.94-7.90 (m, 1H), 7.85-7.73 (m, 3H), 7.62 (dd. J=8.8, 1.6 Hz, 1H), 7.38 (t, J=7.7 Hz, 1H), 7.30-7.24 (m, 2H), 7.18 (d, J=7.4 Hz, 1H), 6.95-6.86 (m, 1H), 5.42 (s, 2H), 5.05 (p, J=6.6 Hz, 1H), 4.03 (q, J=7.1 Hz, 2H), 3.78-3.69 (m, 2H), 3.52 (s, 2H), 1.52 (d, J=6.6 Hz, 6H), 1.17 (t, J=7.1 Hz, 3H), 0.80 (t, J=7.1 Hz, 3H); MS (ES+): 498.20 (M+1); 520.20 (M+Na);

### Step-3: Preparation of ethyl 2-(2-((5-(2-ethoxy-3-(N-hydroxycarbamimidoyl)phenyl)-1-isopropyl-1H-indazol-3-yl)methoxy)phenyl)acetate (273c)

Compound 273c was prepared according to the procedure reported in step-5 of scheme-23 from ethyl 2-(2-((5-(3-cyano-2-ethoxyphenyl)-1-isopropyl-1H-indazol-3-yl)methoxy)phenyl)acetate (273b) (430 mg, 0.864 mmol) in EtOH (10 mL) using hydroxylamine (0.571 mL, 8.64 mmol) to afford after workup and purification using method-E, ethyl 2-(2-((5-(2-ethoxy-3-(N-hydroxycarbamimidoyl)phenyl)-1-isopropyl-1H-indazol-3-yl)methoxy)phenyl)acetate (273c) (250 mg, 54.5% yield) as a white solid; MS (ES+): 531.25 (M+1):

### Step-4: Preparation of ethyl 2-(2-((5-(3-carbamimidoyl-2-ethoxyphenyl)-1-isopropyl-1H-indazol-3-yl)methoxy)phenyl)acetate (273d)

Compound 273d was prepared according to the procedure reported in step-1 of scheme-24 from ethyl 2-(2-((5-(2-ethoxy-3-(N-hydroxycarbamimidoyl)phenyl)-1-isopropyl-1H-indazol-3-yl)methoxy)phenyl)acetate (273c) (250 mg, 0.471 mmol) in EtOH (10 mL) using AcOH (0.027 mL, 0.471 mmol), Raney Nickel (0.471 mmol) and hydrogen (1 atm) to afford after workup and purification using method-AI, ethyl 2-(2-((5-(3-carbamimidoyl-2-ethoxyphenyl)-1-isopropyl-1H-indazol-3-yl)methoxy)phenyl)acetate (273d) (200 mg, 82% yield) as a colorless oil; MS (ES+): 515.25 (M+1).

### Step-5: Preparation of 2-(2-((5-(3-carbamimidoyl-2-ethoxyphenyl)-1-isopropyl-1H-indazol-3-yl)methoxy)phenyl)acetic acid (273e)

Compound 273e was prepared according to the procedure reported step-2 of scheme-1 from ethyl 2-(2-((5-(3-carbamimidoyl-2-ethoxyphenyl)-1-isopropyl-1H-indazol-3-yl)methoxy)phenyl)acetate (273d) (200 mg, 0.389 mmol) in MeOH/THF (6 mL, 1:1) using a solution of LiOH·H₂O (98 mg, 2.332 mmol) in water (2 mL) to afford after workup and purification using method-G, 2-(2-((5-(3-carbamimidoyl-2-ethoxyphenyl)-1-isopropyl-1H-indazol-3-yl)methoxy)phenyl)acetic acid (273e) (85 mg, 45.0% yield) HCl salt as a white solid; ¹H NMR (300 MHz, DMSO-d₆) δ 12.08 (s, 1H, D₂O exchangeable), 9.34 (s, 2H, D₂O exchangeable), 9.20 (s, 2H, D₂O exchangeable), 8.00-7.93 (m, 1H), 7.83 (d, J=8.8 Hz, 1H), 7.73-7.62 (m, 2H), 7.52 (dd, J=7.6, 1.7 Hz, 1H), 7.39 (t, J=7.7 Hz, 1H), 7.30-7.22 (m, 2H), 7.22-7.16 (m, 1H), 6.97-6.87 (m, 1H), 5.44 (s, 2H), 5.16-4.89 (m, 1H), 3.59-3.46 (m, 4H), 1.53 (d, J=6.5 Hz, 6H), 0.98 (t, J=7.0 Hz, 3H); MS (ES+): 487.20 (M+1); MS (ES−): 485.10 (M−1); Analysis calculated for C₂₈H₃₀N₄O₄·HCl·1.5H₂O: C, 61.14; H, 6.23; Cl, 6.44; N, 10.19 Found: C, 61.18; H, 6.29; Cl, 6.36; N, 10.17.

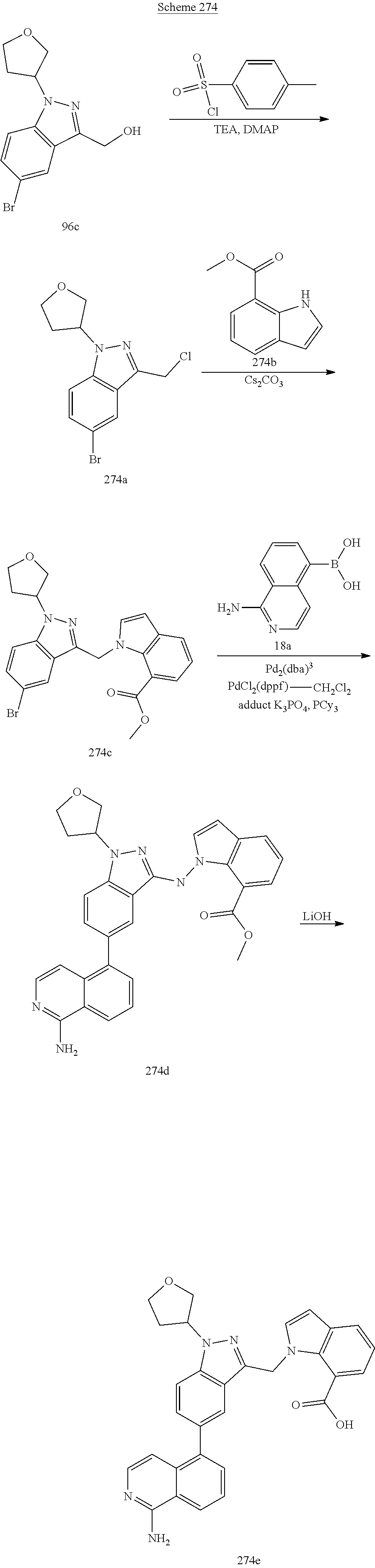

### Preparation of 1-((5-(1-aminoisoquinolin-5-yl)-1-(tetrahydrofuran-3-yl)-1H-indazol-3-yl)methyl)-1H-indole-7-carboxylic acid (274e)

### Step-1: Preparation of 5-bromo-3-(chloromethyl)-1-(tetrahydrofuran-3-yl)-1H-indazole (274a)

Compound 274a was prepared according to the procedure reported in step-1 of scheme-86 from (5-bromo-1-(tetrahydrofuran-3-yl)-1H-indazol-3-yl)methanol (96c) (500 mg, 1.683 mmol) in anhydrous DCM (20 mL) using DMAP (10.28 mg, 0.084 mmol), TEA (0.352 mL, 2.52 mmol) and p-toluenesulfonylchloride (642 mg, 3.37 mmol) to afford after workup and purification using method-AL, 5-bromo-3-(chloromethyl)-1-(tetrahydrofuran-3-yl)-1H-indazole (274a) (220 mg, 41.4% yield) as a clear oil; ¹H NMR (300 MHz, DMSO-d₆) δ 8.12 (d, J=1.8 Hz, 1H), 7.76 (d, J=8.9 Hz, 1H), 7.58 (dd. J=9.0, 1.8 Hz, 1H), 5.57-5.42 (m, 1H), 5.13 (s, 2H), 4.13-3.99 (m, 2H), 3.96-3.77 (m, 2H), 2.46-2.36 (m, 1H), 2.34-2.18 (m, 1H); MS (ES+): 315.00 & 317.00 (M+1).

### Step-2: Preparation of methyl 1-((5-bromo-1-(tetrahydrofuran-3-yl)-1H-indazol-3-yl)methyl)-1H-indole-7-carboxylate (274c)

Compound 274c was prepared according to the procedure reported in step-2 of scheme-86 from 5-bromo-3-(chloromethyl)-1-(tetrahydrofuran-3-yl)-1H-indazole (274a) (220 mg, 0.697 mmol) in DMF (10 mL) using Cs₂CO₃ (681 mg, 2.091 mmol) and 1H-indole-7-carboxylate (274b) (366 mg, 2.091 mmol) to afford after workup and purification using method-P, methyl 1-((5-bromo-1-(tetrahydrofuran-3-yl)-1H-indazol-3-yl)methyl)-1H-indole-7-carboxylate (274c) (210 mg, 66.3% yield) as a white solid; ¹H NMR (300 MHz, DMSO-d₆) δ 7.89 (d, J=3.2 Hz, 1H), 7.80-7.74 (m, 1H), 7.60 (d, J=8.9 Hz, 1H), 7.45-7.36 (m, 3H), 7.04 (t, J=7.6 Hz, 1H), 6.67 (d, J=3.2 Hz, 1H), 5.91 (s, 2H), 5.46-5.30 (m, 1H), 4.07-4.01 (m, 2H), 3.89 (s, 3H), 3.87-3.78 (m, 2H), 2.42-2.13 (m, 2H); MS (ES+): 454.10 & 456.10 (M+1).

### Step-3: Preparation of methyl 1-((5-(1-aminoisoquinolin-5-yl)-1-(tetrahydrofuran-3-yl)-1H-indazol-3-yl)methyl)-1H-indole-7-carboxylate (274d)

Compound 274d was prepared according to the procedure reported in step-5 of scheme-1 from methyl 1-((5-bromo-1-(tetrahydrofuran-3-yl)-1H-indazol-3-yl)methyl)-1H-indole-7-carboxylate (274c) (100 mg, 0.22 mmol) in dioxane/THF (6 mL, 1:1) using (1-aminoisoquinolin-5-yl)boronic acid (18a) (62.1 mg, 0.33 mmol), K₃PO₄ (2 M aqueous solution, 0.44 mL, 0.88 mmol). PCy₃ (12.35 mg, 0.044 mmol). PdCl₂(dppf)-CH₂Cl₂ adduct (17.98 mg, 0.022 mmol) and Pd₂(dba)₃ (20.16 mg, 0.022 mmol) to afford after workup and purification using method-AM, methyl 1-((5-(1-aminoisoquinolin-5-yl)-1-(tetrahydrofuran-3-yl)-1H-indazol-3-yl)methyl)-1H-indole-7-carboxylate (274d) (70 mg, 61.4% yield) as a clear oil; MS (ES+): 518.20 (M+1).

### Step-4: Preparation of 1-((5-(1-aminoisoquinolin-5-yl)-1-(tetrahydrofuran-3-yl)-1H-indazol-3-yl)methyl)-1H-indole-7-carboxylic acid (274e)

Compound 274e was prepared according to the procedure reported in step-2 of scheme-1 from methyl 1-((5-(1-aminoisoquinolin-5-yl)-1-(tetrahydrofuran-3-yl)-1H-indazol-3-yl)methyl)-1H-indole-7-carboxylate (274d) (70 mg, 0.135 mmol) in MeOH/THF (6 mL, 1:1) using a solution of LiOH·H₂O (34.1 mg, 0.811 mmol) in water (2 mL) to afford after workup and purification using method-G, 1-((5-(1-aminoisoquinolin-5-yl)-1-(tetrahydrofuran-3-yl)-1H-indazol-3-yl)methyl)-1H-indole-7-carboxylic acid (274e) (20 mg, 29.4% yield) HCl salt as a white solid; ¹H NMR (300 MHz, DMSO-d₆) δ 13.09 (s, 1H, D₂O exchangeable), 9.11 (s, 2H, D₂O exchangeable), 8.58 (d, J=8.4 Hz, 1H), 7.87-7.67 (m, 5H), 7.60 (d, J=7.1 Hz, 1H), 7.49 (d, J=7.3 Hz, 1H), 7.34 (d, J=8.7 Hz, 1H), 7.28 (s, 1H), 7.04 (t, J=7.6 Hz, 1H), 6.66 (d, J=7.2 Hz, 1H), 6.58 (d, J=3.2 Hz, 1H), 6.09-5.98 (m, 2H), 5.52-5.34 (m, 1H), 4.15-4.02 (m, 2H), 3.93-3.86 (m, 2H), 2.40-2.33 (m, 2H); MS (ES+): 504.20 (M+1); (ES−): 502.10 (M−1).

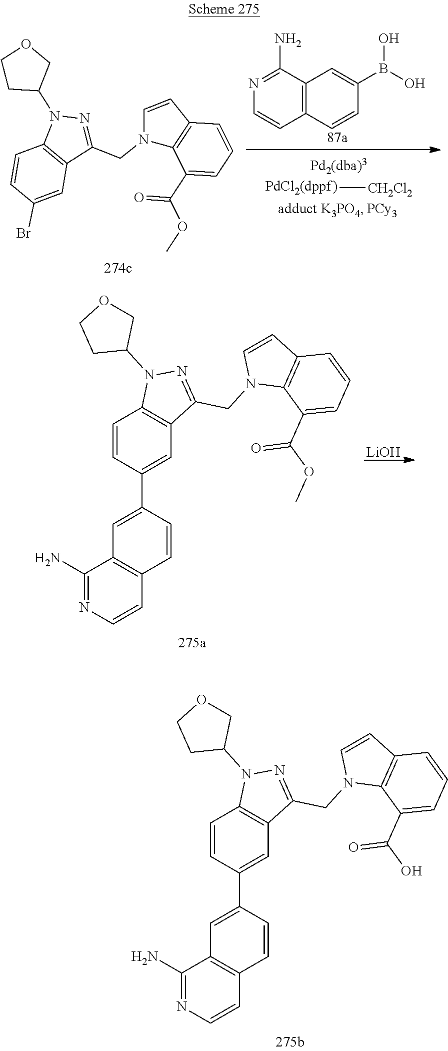

### Preparation of 1-((5-(1-aminoisoquinolin-7-yl)-1-(tetrahydrofuran-3-yl)-1H-indazol-3-yl)methyl)-1H-indole-7-carboxylic acid (275b)

### Step-1: Preparation of methyl 1-((5-(1-aminoisoquinolin-7-yl)-1-(tetrahydrofuran-3-yl)-1H-indazol-3-yl)methyl)-1H-indole-7-carboxylate (275a)

Compound 275a was prepared according to the procedure reported in step-5 of scheme-1 from methyl 1-((5-bromo-1-(tetrahydrofuran-3-yl)-1H-indazol-3-yl)methyl)-1H-indole-7-carboxylate (274c) (100 mg, 0.22 mmol) in dioxane/THF (6 mL, 1:1) using 1-aminoisoquinolin-7-ylboronic acid (87a) (62.1 mg, 0.330 mmol), K₃PO₄ (2 M aqueous solution, 0.440 mL, 0.880 mmol), PCy₃ (12.35 mg, 0.044 mmol). PdCl₂(dppf)-CH₂Cl₂ adduct (17.98 mg, 0.022 mmol) and Pd₂(dba)₃ (20.16 mg, 0.022 mmol) to afford after workup and purification using method-AM, methyl 1-((5-(1-aminoisoquinolin-7-yl)-1-(tetrahydrofuran-3-yl)-1H-indazol-3-yl)methyl)-1H-indole-7-carboxylate (275a) (70 mg, 61.4% yield) as a clear oil; MS (ES+): 518.20 (M+1).

### Step-2: Preparation of 1-((5-(1-aminoisoquinolin-7-yl)-1-(tetrahydrofuran-3-yl)-1H-indazol-3-yl)methyl)-1H-indole-7-carboxylic acid (275b)

Compound 275b was prepared according to the procedure reported in step-2 of scheme-1 from methyl 1-((5-(1-aminoisoquinolin-7-yl)-1-(tetrahydrofuran-3-yl)-1H-indazol-3-yl)methyl)-1H-indole-7-carboxylate (275a) (70 mg, 0.135 mmol) in MeOH/THF (6 mL, 1:1) using a solution of LiOH·H₂O (34.1 mg, 0.811 mmol) in water (2 mL) to afford after workup and purification using method-G, 1-((5-(1-aminoisoquinolin-7-yl)-1-(tetrahydrofuran-3-yl)-1H-indazol-3-yl)methyl)-1H-indole-7-carboxylic acid (275b) (28 mg, 41.1% yield) HCl salt as a white solid; 3H NMR (300 MHz, DMSO-d₆) δ 13.14 (s, 1H, D₂O exchangeable), 9.19 (s, 2H, D₂O exchangeable), 8.93 (s, 1H), 8.18-8.08 (m, 1H), 8.04 (d, J=8.5 Hz, 1H), 7.95-7.84 (m, 3H), 7.80 (d, J=8.9 Hz, 1H), 7.77-7.71 (m, 1H), 7.68 (d, J=7.0 Hz, 1H), 7.56-7.49 (m, 1H), 7.26 (d, J=7.0 Hz, 1H), 7.04 (t, J=7.6 Hz, 1H), 6.62 (d, J=3.2 Hz, 1H), 6.15-6.07 (m, 2H), 5.50-5.31 (m, 1H), 4.13-3.98 (m, 2H), 3.91-3.77 (m, 2H), 2.39-2.26 (m, 2H); MS (ES+): 504.20 (M+1); (ES−): 502.10 (M−1).

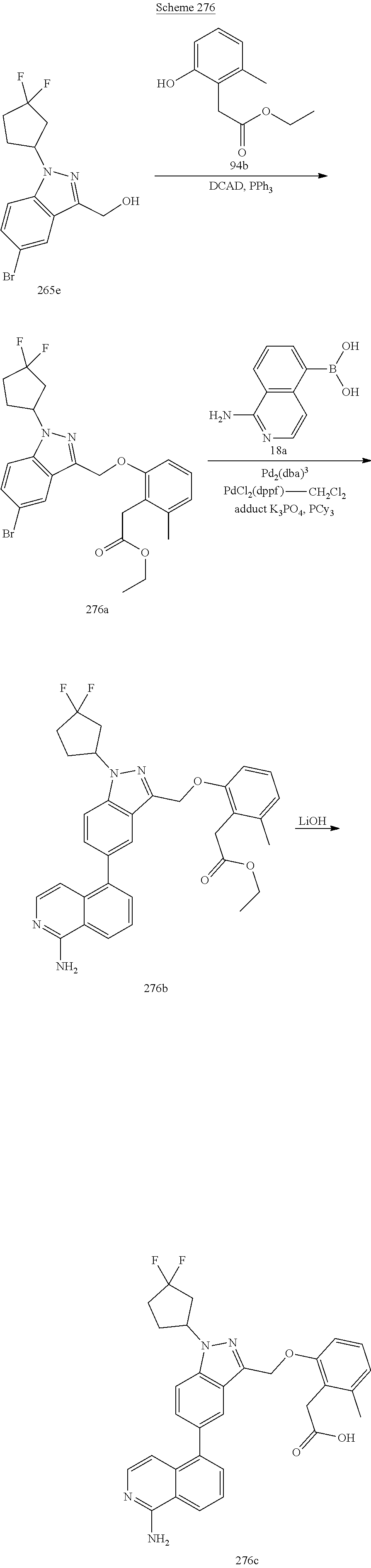

### Preparation of 2-(2-((5-(1-aminoisoquinolin-5-yl)-1-(3,3-difluorocyclopentyl)-1H-indazol-3-yl)methoxy)-6-methylphenyl)acetic acid (276c)

### Step-1: Preparation of ethyl 2-(2-((5-bromo-1-(3,3-difluorocyclopentyl)-1H-indazol-3-yl)methoxy)-6-methylphenyl)acetate (276a)

Compound 276a was prepared according to the procedure reported in step-2 of scheme-2 from (5-bromo-1-(3,3-difluorocyclopentyl)-1H-indazol-3-yl)methanol (265e) (290 mg, 0.876 mmol) in DCM (10 mL) using ethyl 2-(2-hydroxy-6-methylphenyl)acetate (94b) (179 mg, 0.920 mmol), PPh₃ (276 mg, 1.051 mmol) and a solution of DCAD (386 mg, 1.051 mmol) in DCM (10 mL) to afford after workup and purification using method-N, ethyl 2-(2-((5-bromo-1-(3,3-difluorocyclopentyl)-1H-indazol-3-yl)methoxy)-6-methylphenyl)acetate (276a) (250 mg, 56.3% yield) as a white oil; H NMR (300 MHz, DMSO-d₆) δ 7.95 (d, J=1.8 Hz, 1H), 7.76 (d, J=8.9 Hz, 1H), 7.57 (dd, J=8.9, 1.8 Hz, 1H), 7.21-7.12 (m, 1H), 7.08 (d, J=8.2 Hz, 1H), 6.82 (d, J=7.3 Hz, 1H), 5.54-5.40 (m, 1H), 5.37 (s, 2H), 3.96 (q, J=7.1 Hz, 2H), 3.59 (s, 2H), 2.92-2.55 (m, 2H), 2.49-2.20 (m, 4H), 2.20 (s, 3H), 1.00 (t, J=7.1 Hz, 3H); MS (ES+): 507.10 & 509.10 (M+1).

### Step-2: Preparation of ethyl 2-(2-((5-(1-aminoisoquinolin-5-yl)-1-(3,3-difluorocyclopentyl)-1H-indazol-3-yl)methoxy)-6-methylphenyl)acetate (276b)

Compound 276b was prepared according to the procedure reported in step-S of scheme-1 from ethyl 2-(2-((5-bromo-1-(3,3-difluorocyclopentyl)-1H-indazol-3-yl)methoxy)-6-methylphenyl)acetate (276a) (125 mg, 0.246 mmol) in dioxane/THF (6 mL, 1:1) using (1-aminoisoquinolin-5-yl)boronic acid (18a) (69.5 mg, 0.37 mmol), K₃PO₄ (2 M aqueous solution, 0.493 mL, 0.985 mmol), PCy₃ (13.82 mg, 0.049 mmol), PdCl₂(dppf)-CH₂Cl₂ adduct (20.12 mg, 0.025 mmol) and Pd₂(dba)₃ (22.56 mg, 0.025 mmol) to afford after workup and purification using method-AM, ethyl 2-(2-((5-(1-aminoisoquinolin-5-yl)-1-(3,3-difluorocyclopentyl)-1H-indazol-3-yl)methoxy)-6-methylphenyl)acetate (276b) (80 mg, 56.9% yield) as a clear oil; MS (ES+): 571.20 (M+1).

### Step-3: Preparation of 2-(2-((5-(1-aminoisoquinolin-5-yl)-1-(3,3-difluorocyclopentyl)-1H-indazol-3-yl)methoxy)-6-methylphenyl)acetic acid (276c)

Compound 276c was prepared according to the procedure reported in step-2 of scheme-1 from ethyl 2-(2-((5-(1-aminoisoquinolin-5-yl)-1-(3,3-difluorocyclopentyl)-1H-indazol-3-yl)methoxy)-6-methylphenyl)acetate (276b) (80 mg, 0.14 mmol) in MeOH/THF (6 mL, 1:1) using a solution of LiOH·H₂O (35.3 mg, 0.841 mmol) in water (2 mL) to afford after workup and purification using method-G, 2-(2-((5-(1-aminoisoquinolin-5-yl)-1-(3,3-difluorocyclopentyl)-1H-indazol-3-yl)methoxy)-6-methylphenyl)acetic acid (276c) (25 mg, 32.9% yield) HCl salt as a white solid; ¹H NMR (300 MHz, DMSO-d₆) δ 13.26 (s, 1H, D₂O exchangeable), 12.02 (s, 1H, D₂O exchangeable), 9.15 (s, 2H, D₂O exchangeable), 8.62 (d, J=8.2 Hz, 1H), 7.99-7.79 (m, 4H), 7.63 (d, J=7.2 Hz, 1H), 7.51 (dd, 1H), 7.20-7.04 (m, 2H), 6.97 (d, J=7.2 Hz, 1H), 6.86-6.71 (m, 1H), 5.64-5.48 (m, 1H), 5.43 (s, 2H), 3.54 (s, 2H), 2.94-2.59 (m, 2H), 2.48-2.19 (m, 4H), 2.17 (s, 3H); 1ºF NMR (282 MHz, DMSO-d₆) δ −87.28-89.83 (m); MS (ES+): 543.20 (M+1); (ES−): 541.20 (M−1).

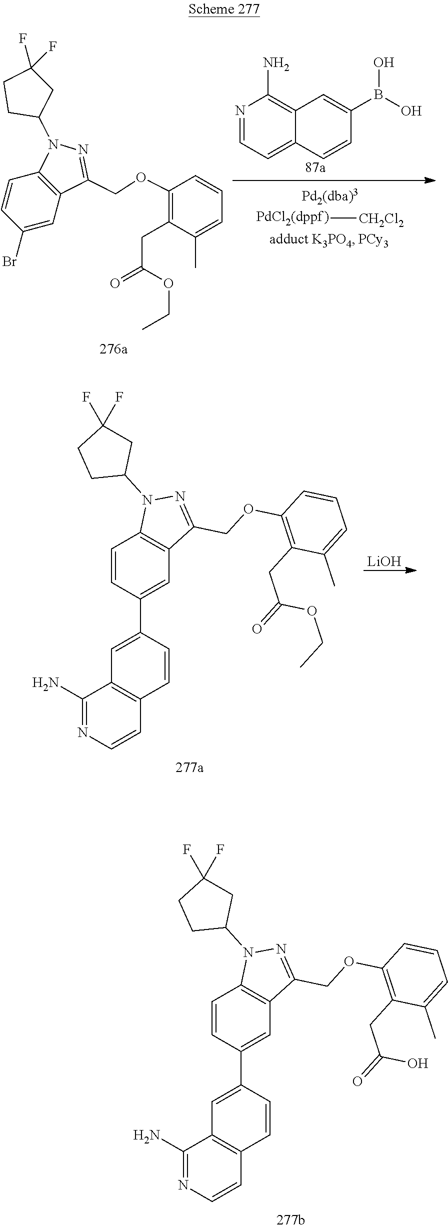

### Preparation of 2-(2-((5-(1-aminoisoquinolin-7-yl)-1-(3,3-difluorocyclopentyl)-1H-indazol-3-yl)methoxy)-6-methylphenyl)acetic acid (277b)

### Step-1: Preparation of ethyl 2-(2-((5-(1-aminoisoquinolin-7-yl)-1-(3,3-difluorocyclopentyl)-1H-indazol-3-yl)methoxy)-6-methylphenyl)acetate (277a)

Compound 277a was prepared according to the procedure reported in step-5 of scheme-1 from ethyl 2-(2-((5-bromo-1-(3,3-difluorocyclopentyl)-1H-indazol-3-yl)methoxy)-6-methylphenyl)acetate (276a) (125 mg, 0.246 mmol) in dioxane/THF (6 mL, 1:1) using 1-aminoisoquinolin-7-ylboronic acid (87a) (69.5 mg, 0.37 mmol), K₃PO₄ (2 M aqueous solution, 0.493 mL, 0.985 mmol), PCy₃ (13.82 mg, 0.049 mmol), PdCl₂(dppf)-CH₂Cl₂ adduct (20.12 mg, 0.025 mmol) and Pd₂(dba)₃ (22.56 mg, 0.025 mmol) to afford after workup and purification using method-AM, ethyl 2-(2-((5-(1-aminoisoquinolin-7-yl)-1-(3,3-difluorocyclopentyl)-1H-indazol-3-yl)methoxy)-6-methylphenyl)acetate (277a) (80 mg, 56.9% yield) as a clear oil; MS (ES+): 571.20 (M+1).

### Step-2: Preparation of 2-(2-((5-(1-aminoisoquinolin-7-yl)-1-(3,3-difluorocyclopentyl)-1H-indazol-3-yl)methoxy)-6-methylphenyl)acetic acid (277b)

Compound 277b was prepared according to the procedure reported in step-2 of scheme-1 from ethyl 2-(2-((5-(1-aminoisoquinolin-7-yl)-1-(3,3-difluorocyclopentyl)-1H-indazol-3-yl)methoxy)-6-methylphenyl)acetate (277a) (80 mg, 0.14 mmol) in MeOH/THF (6 mL, 1:1) using a solution of LiOH·H₂O (35.3 mg, 0.841 mmol) in water (2 mL) to afford after workup and purification using method-G, 2-(2-((5-(1-aminoisoquinolin-7-yl)-1-(3,3-difluorocyclopentyl)-1H-indazol-3-yl)methoxy)-6-methylphenyl)acetic acid (277b) (20 mg, 26.3% yield) HCl salt as a white solid; ¹H NMR (300 MHz, DMSO-d₆) & 13.18 (s, 1H, D₂O exchangeable), 12.12 (s, 1H, D₂O exchangeable), 9.11 (s, 2H, D₂O exchangeable), 8.94 (s, 1H), 8.39 (dd, 1H), 8.31 (d, 1H), 8.09-7.99 (m, 2H), 7.99-7.91 (m, 1H), 7.69 (d, J=6.9 Hz, 1H), 7.28 (d, J=7.0 Hz, 1H), 7.19-7.08 (m, 2H), 6.87-6.74 (m, 1H), 5.61-5.50 (m, 1H), 5.47 (s, 2H), 3.58 (s, 2H), 2.94-2.65 (m, 2H), 2.48-2.21 (m, 4H), 2.19 (s, 3H); 1ºF NMR (282 MHz, DMSO-d₆) δ −85.63-91.83 (m); MS (ES+): 543.20 (M+1); (ES−): 541.20 (M−1).

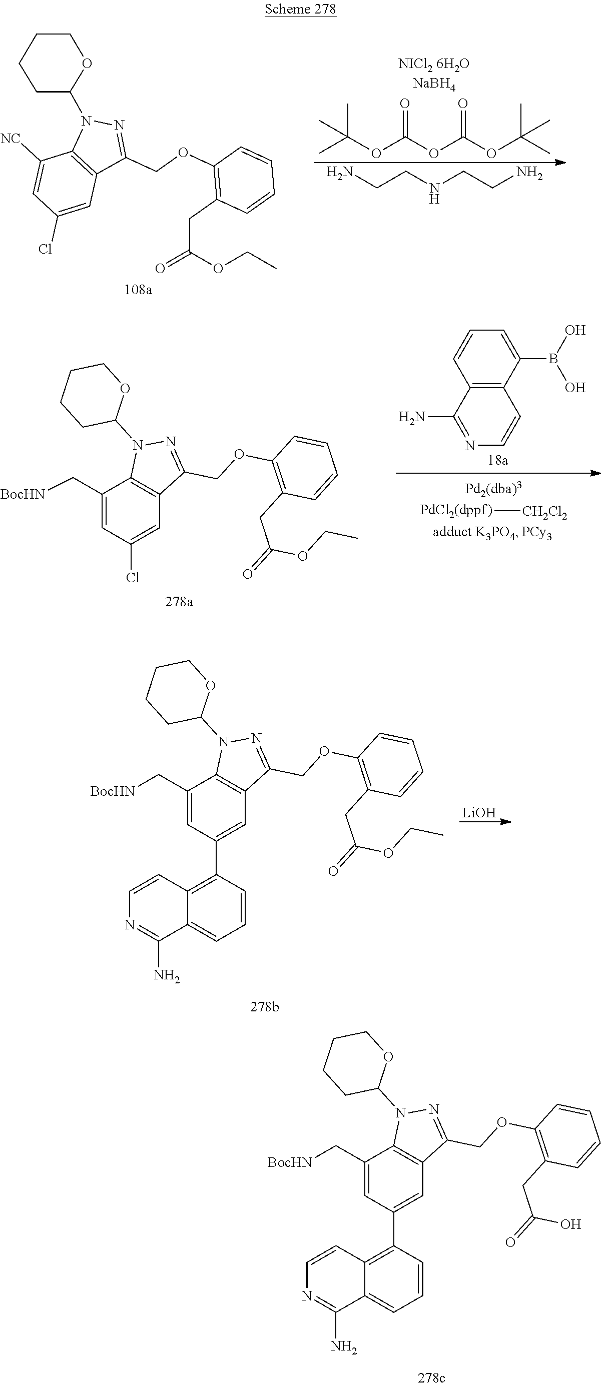

### Preparation of 2-(2-((5-(1-aminoisoquinolin-5-yl)-7-(((tert-butoxycarbonyl)amino)methyl)-1-(tetrahydro-2H-pyran-2-yl)-1H-indazol-3-yl)methoxy)phenyl)acetic acid (278c)

### Step-1: Preparation of ethyl 2-(2-((7-((tert-butoxycarbonylamino)methyl)-5-chloro-1-(tetrahydro-2H-pyran-2-yl)-1H-indazol-3-yl)methoxy)phenyl)acetate (278a)

To a stirred solution of ethyl 2-(2-((5-chloro-7-cyano-1-(tetrahydro-2H-pyran-2-yl)-1H-indazol-3-yl)methoxy)phenyl)acetate (108a) (440 mg, 0.969 mmol) and (Boc)₂O (423 mg, 1.939 mmol) in anhydrous ethanol (10 mL) at 0° C. was added NiCl_{2.6}H₂O (57.6 mg, 0.242 mmol) followed by NaBH₄ (110 mg, 2.91 mmol) (in small portions over a period of 10 min. The reaction was exothermic and effervescent) and stirred for 24 h. The reaction was quenched with N1-(2-aminoethyl)ethane-1,2-diamine (0.209 mL, 1.939 mmol) allowed to stir for 1 h and concentrated in vacuum. To the residue was added brine (100 mL) and extracted with ethyl acetate (2×150 mL). The combined organic layer was dried, filtered and concentrated in vacuum. The residue obtained was purified using method-BI to furnish ethyl 2-(2-((7-((tert-butoxycarbonylamino)methyl)-5-chloro-1-(tetrahydro-2H-pyran-2-yl)-1H-indazol-3-yl)methoxy)phenyl)acetate (278a) (400 mg, 73.9% yield) as a white solid; MS (ES+): 580.20 & 582.20 (M+Na).

### Step-2: Preparation of ethyl 2-(2-((5-(1-aminoisoquinolin-5-yl)-7-((tert-butoxycarbonylamino)methyl)-1-(tetrahydro-2H-pyran-2-yl)-1H-indazol-3-yl)methoxy)phenyl)acetate (278b)

Compound 278b was prepared according to the procedure reported in step-5 of scheme-1 from ethyl 2-(2-((7-((tert-butoxycarbonylamino)methyl)-5-chloro-1-(tetrahydro-2H-pyran-2-yl)-1H-indazol-3-yl)methoxy)phenyl)acetate (278a) (400 mg, 0.717 mmol) in dioxane/THF (6 mL, 1:1) using (1-aminoisoquinolin-5-yl)boronic acid (18a) (202 mg, 1.075 mmol), K₃PO₄ (2 M aqueous solution, 1.434 mL, 2.87 mmol), PCy₃ (40.2 mg, 0.143 mmol), PdCl₂(dppf)-CH₂Cl₂ adduct (58.5 mg, 0.072 mmol) and Pd₂(dba)₃ (65.6 mg, 0.072 mmol) to afford after workup and purification using method-AM, ethyl 2-(2-((5-(1-aminoisoquinolin-5-yl)-7-((tert-butoxycarbonylamino)methyl)-1-(tetrahydro-2H-pyran-2-yl)-1H-indazol-3-yl)methoxy)phenyl)acetate (278b) (200 mg, 41.9% yield) as a clear oil; MS (ES+): 666.30 (M+1);

### Step-3: Preparation of 2-(2-((5-(1-aminoisoquinolin-5-yl)-7-((tert-butoxycarbonylamino)methyl)-1-(tetrahydro-2H-pyran-2-yl)-1H-indazol-3-yl)methoxy)phenyl)acetic acid (278c)

Compound 278c was prepared according to the procedure reported in step-2 of scheme-1 from ethyl 2-(2-((5-(1-aminoisoquinolin-5-yl)-7-((tert-butoxycarbonylamino)methyl)-1-(tetrahydro-2H-pyran-2-yl)-1H-indazol-3-yl)methoxy)phenyl)acetate (278b) (80 mg, 0.120 mmol) in MeOH/THF (6 ml, 1:1) using a solution of LiOH. H₂O (30.3 mg, 0.721 mmol) in water (2 mL) to afford after workup and purification using method-G, 2-(2-((5-(1-aminoisoquinolin-5-yl)-7-((tert-butoxycarbonylamino)methyl)-1-(tetrahydro-2H-pyran-2-yl)-1H-indazol-3-yl)methoxy)phenyl)acetic acid (278c) (35 mg, 45.7% yield) HCl salt as a white solid; ¹H NMR (300 MHz, DMSO-d₆) δ 13.29 (s, 1H, D₂O exchangeable), 12.01 (s, 1H, D₂O exchangeable), 9.19 (s, 2H, D₂O exchangeable), 8.63 (d, J=8.2 Hz, 1H), 7.98-7.78 (m, 3H), 7.59 (d, J=7.2 Hz, 1H), 7.57-7.47 (m, 1H, D₂O exchangeable), 7.36 (d, 1H), 7.24 (d, J=4.1 Hz, 2H), 7.17 (d, J=7.4 Hz, 1H), 7.03 (d, J=7.2 Hz, 1H), 6.96-6.85 (m, 1H), 5.88 (d, J=9.1 Hz, 1H), 5.44 (s, 2H), 4.82 (dd, J=15.5, 6.5 Hz, 1H), 4.69-4.36 (m, 1H), 4.03-3.71 (m, 2H), 3.47 (s, 2H), 2.63-2.53 (m, 1H), 2.19-2.01 (m, 2H), 1.85-1.50 (m, 3H), 1.39 (s, 9H); MS (ES+): 638.30 (M+1); (ES−): 636.20 (M−1).

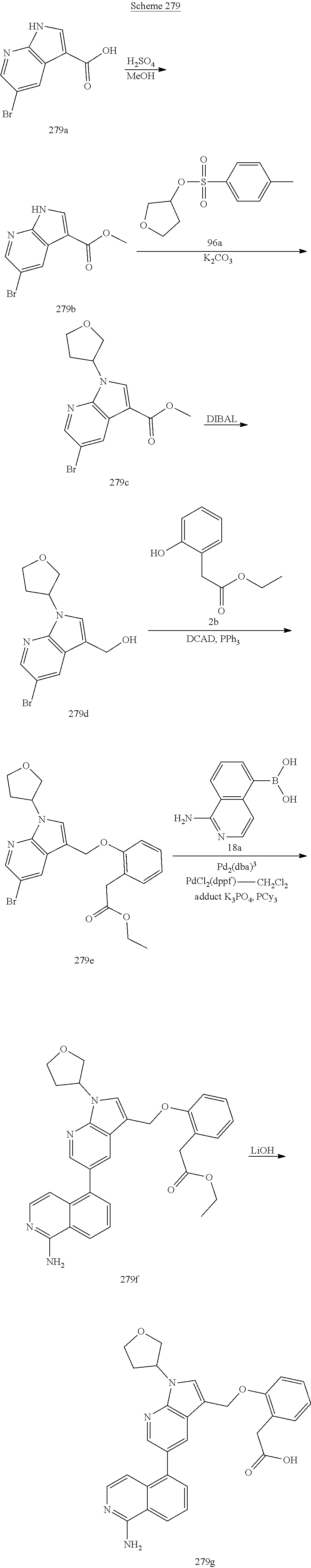

### Preparation of 2-(2-((5-(1-aminoisoquinolin-5-yl)-1-(tetrahydrofuran-3-yl)-1H-pyrrolo[2,3-b]pyridin-3-yl)methoxy)phenyl)acetic acid (279g)

### Step-1: Preparation of methyl 5-bromo-1H-pyrrolo[2,3-b]pyridine-3-carboxylate (279b)

To a solution of 5-bromo-1H-pyrrolo[2,3-b]pyridine-3-carboxylic acid (279a) (5 g, 20.74 mmol; CAS #849068-61-7) in MeOH (50 mL) was added sulfuric acid (1.106 mL, 20.74 mmol) and heated at reflux for 48 h. The reaction mixture was cooled to room temperature concentrated in vacuum and residue obtained was purified using method-S to afford methyl 5-bromo-1H-pyrrolo[2,3-b]pyridine-3-carboxylate (279b) (4.6 g, 87% yield) as a white solid; ¹H NMR (300 MHz, DMSO-d₆) δ 12.79 (s, 1H), 8.41 (s, 2H), 8.31 (d, J=3.1 Hz, 1H), 3.83 (s, 3H); MS (ES+): 254.95 & 256.95 (M+1); (ES−): 252.90 & 254.90 (M−1).

### Step-2: Preparation of methyl 5-bromo-1-(tetrahydrofuran-3-yl)-1H-pyrrolo[2,3-b]pyridine-3-carboxylate (279c)

Compound 279c was prepared according to the procedure reported in step-3 of scheme-106 from methyl 5-bromo-1H-pyrrolo[2,3-b]pyridine-3-carboxylate (279b) (2.5 g, 9.80 mmol) in DMF (30 mL) using K₂CO₃ (2.71 g, 19.60 mmol), tetrahydrofuran-3-yl 4-methylbenzenesulfonate (96a) (4.75 g, 19.60 mmol) and heating at 60° C. overnight to afford after workup and purification using method-AO, methyl 5-bromo-1-(tetrahydrofuran-3-yl)-1H-pyrrolo[2,3-b]pyridine-3-carboxylate (279c) (3 g, 94% yield) as a white solid; MS (ES+): 325.00 & 327.00 (M+1).

### Step-3: Preparation of (5-bromo-1-(tetrahydrofuran-3-yl)-1H-pyrrolo[2,3-b]pyridin-3-yl)methanol (279d)

Compound 279d was prepared according to the procedure reported in step-1 of scheme-2 from methyl 5-bromo-1-(tetrahydrofuran-3-yl)˜1H-pyrrolo[2,3-b]pyridine-3-carboxylate (279c) (2 g, 6.15 mmol) in DCM (30 mL) using DIBAL (1M solution in DCM, 15.38 mL, 15.38 mmol) to afford after workup and purification using method-D, (5-bromo-1-(tetrahydrofuran-3-yl)-1H-pyrrolo[2,3-b]pyridin-3-yl)methanol (279d) (300 mg, 16.41% yield) as a light yellow oil; ¹H NMR (300 MHz, DMSO-d₆) δ 8.32 (d, J=2.3 Hz, 1H), 8.25 (d, J=2.2 Hz, 1H), 7.54 (s, 1H), 5.52-5.31 (m, 1H), 5.03-4.94 (m, 1H), 4.61 (d, J=5.4 Hz, 2H), 4.16-4.04 (m, 1H), 4.03-3.91 (m, 1H), 3.91-3.78 (m, 2H), 2.49-2.41 (m, 1H), 2.20-2.02 (m, 1H); MS (ES+): 297.00 & 299.00 (M+1).

### Step-4: Preparation of ethyl 2-(2-((5-bromo-1-(tetrahydrofuran-3-yl)-1H-pyrrolo[2,3-b]pyridin-3-yl)methoxy)phenyl)acetate (279e)

Compound 279e was prepared according to the procedure reported in step-2 of scheme-2 from (5-bromo-1-(tetrahydrofuran-3-yl)-1H-pyrrolo[2,3-b]pyridin-3-yl)methanol (279d) (300 mg, 1.010 mmol) in DCM (10 mL) using ethyl 2-(2-hydroxyphenyl)acetate (2b) (218 mg, 1.212 mmol). PPh₃ (397 mg, 1.514 mmol) a solution of DCAD (556 mg, 1.514 mmol) in DCM (10 mL) to afford after workup and purification using method-S, ethyl 2-(2-((5-bromo-1-(tetrahydrofuran-3-yl)-1H-pyrrolo[2,3-b]pyridin-3-yl)methoxy)phenyl)acetate (279e) (180 mg, 38.8% yield) as a white oil; MS (ES+): 459.10 & 461.10 (M+1); (ES+): 481.10 & 483.00 (M+Na).

### Step-5: Preparation of ethyl 2-(2-((5-(1-aminoisoquinolin-5-yl)-1-(tetrahydrofuran-3-yl)-1H-pyrrolo[2,3-b]pyridin-3-yl)methoxy)phenyl)acetate (279f)

Compound 279f was prepared according to the procedure reported in step-5 of scheme-1 from ethyl 2-(2-((5-bromo-1-(tetrahydrofuran-3-yl)-1H-pyrrolo[2,3-b]pyridin-3-yl)methoxy)phenyl)acetate (279e) (180 mg, 0.392 mmol) in dioxane/THF (6 mL, 1:1) using (1-aminoisoquinolin-5-yl)boronic acid (18a) (111 mg, 0.588 mmol), K₃PO₄ (2 M aqueous solution, 0.784 mL, 1.567 mmol), PCy₃ (21.98 mg, 0.078 mmol). PdCl₂(dppf)-CH₂Cl₂Adduct (32.0 mg, 0.039 mmol) and Pd₂(dba)₃ (35.9 mg, 0.039 mmol) to afford after workup and purification using method-AM, ethyl 2-(2-((5-(1-aminoisoquinolin-5-yl)-1-(tetrahydrofuran-3-yl)-1H-pyrrolo[2,3-b]pyridin-3-yl)methoxy)phenyl)acetate (279f) (55 mg, 26.9% yield) as a clear oil; MS (ES+): 523.25 (M+1).

### Step-6: Preparation of 2-(2-((5-(1-aminoisoquinolin-5-yl)-1-(tetrahydrofuran-3-yl)-1H-pyrrolo[2,3-b]pyridin-3-yl)methoxy)phenyl)acetic acid (279g)

Compound 279g was prepared according to the procedure reported in step-2 of scheme-1 from ethyl 2-(2-((5-(1-aminoisoquinolin-5-yl)-1-(tetrahydrofuran-3-yl)-1H-pyrrolo[2,3-b]pyridin-3-yl)methoxy)phenyl)acetate (279f) (50 mg, 0.096 mmol) in MeOH/THF (4 mL, 1:1) using a solution of LiOH·H₂O (12.04 mg, 0.287 mmol) in water (1 mL) to afford after workup and purification using method-G, 2-(2-((5-(1-aminoisoquinolin-5-yl)-1-(tetrahydrofuran-3-yl)-1H-pyrrolo[2,3-b]pyridin-3-yl)methoxy)phenyl)acetic acid (279g) (6 mg, 12.68% yield) HCl salt as a white solid; ¹H NMR (300 MHz, DMSO-d₆) δ 12.09 (s, 1H, D₂O exchangeable), 8.34 (d, J=2.0 Hz, 1H), 8.25 (d, J=8.2 Hz, 1H), 8.11 (d, J=2.1 Hz, 1H), 7.84-7.74 (m, 2H), 7.67-7.60 (m, 1H), 7.59-7.53 (m, 1H), 7.27-7.14 (m, 3H), 6.93 (s, 2H, D₂O exchangeable), 6.91-6.85 (m, 1H), 6.79 (d, J=6.2 Hz, 1H), 5.65-5.48 (m, 1H), 5.29 (s, 2H), 4.20-3.99 (m, 2H), 3.99-3.83 (m, 2H), 3.50 (s, 2H), 2.64-2.52 (m, 1H), 2.30-2.06 (m, 1H); MS (ES+): 495.20 (M+1); (ES−): 493.10 (M−1).

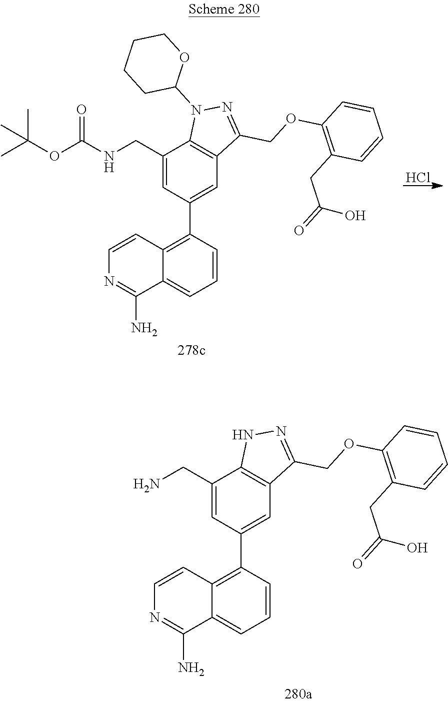 [SMILES: CC(C)(C)OC(=O)NCc1cc(-c2cccc3c(N)nccc23)cc2c(COc3ccccc3CC(=O)O)nn(C3CCCCO3)c12 ; Cl ; NCc1cc(-c2cccc3c(N)nccc23)cc2c(COc3ccccc3CC(=O)O)n[nH]c12]

### Preparation of 2-(2-((5-(1-aminoisoquinolin-5-yl)-7-(aminomethyl)-1H-indazol-3-yl)methoxy)phenyl)acetic acid (280a)

To a solution of 2-(2-((5-(1-aminoisoquinolin-5-yl)-7-((tert-butoxycarbonylamino)methyl)-1-(tetrahydro-2H-pyran-2-yl)-1H-indazol-3-yl)methoxy)phenyl)acetic acid (278c) (40 mg, 0.063 mmol) in dioxane (5 mL) was added HCl (4 M HCl in dioxane) (0.157 mL, 0.627 mmol) and stirred at RT for 15 h. The reaction mixture was cooled to room temperature concentrated in vacuum and residue obtained was purified using method-G to afford 2-(2-((5-(1-aminoisoquinolin-5-yl)-7-(aminomethyl)-1H-indazol-3-yl)methoxy)phenyl)acetic acid (280a) (20 mg, 70.3% yield) HCl salt as a white solid; ¹H NMR (300 MHz, DMSO-d₆) δ 13.71 (s, 1H, D₂O exchangeable), 13.38 (s, 1H, D₂O exchangeable), 12.04 (s, 1H, D₂O exchangeable), 9.24 (s, 3H, D₂O exchangeable), 8.71-8.48 (m, 4H, 3H D₂O exchangeable), 8.01-7.84 (m, 3H), 7.69-7.58 (m, 2H), 7.30-7.21 (m, 2H), 7.17 (d, J=7.4 Hz, 1H), 7.07 (d, J=7.3 Hz, 1H), 6.95-6.83 (m, 1H), 5.49 (s, 2H), 4.52-4.40 (m, 2H), 3.47 (s, 2H); MS (ES+): 454.20 (M+1).

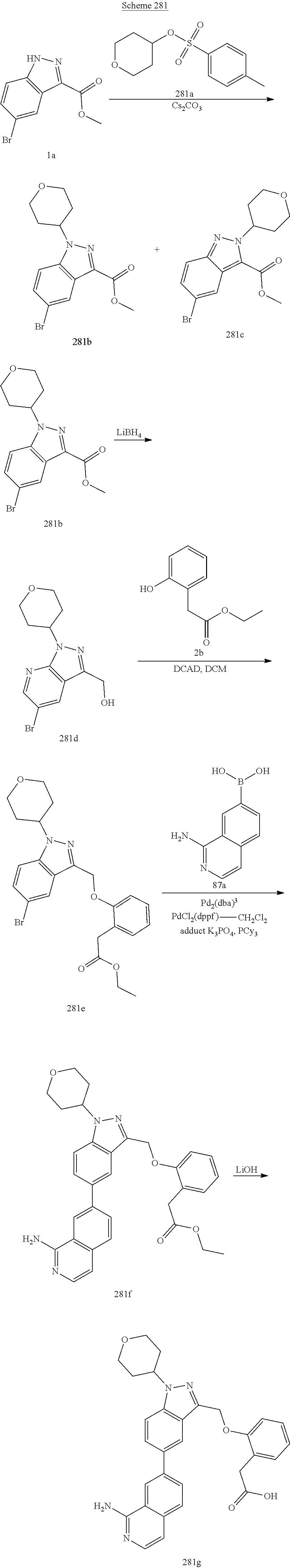

### Preparation of 2-(2-((5-(1-aminoisoquinolin-7-yl)-1-(tetrahydro-2H-pyran-4-yl)-1H-indazol-3-yl)methoxy)phenyl)acetic acid (281g)

### Step-1: Preparation of methyl 5-bromo-1-(tetrahydro-2H-pyran-4-yl)-1H-indazole-3-carboxylate (281b) and methyl 5-bromo-2-(tetrahydro-2H-pyran-4-yl)-2H-indazole-3-carboxylate (281c)

Compounds 281b and 281c were prepared according to the procedure reported in step-2 of scheme-86 from methyl 5-bromo-1H-indazole-3-carboxylate (1a) (10.17 g, 39.9 mmol) in DMF (60 mL) using Cs₂CO₃ (26.0 g, 80 mmol) and tetrahydro-2H-pyran-4-yl 4-methylbenzenesulfonate (281a) (10.22 g, 39.9 mmol; CAS #97986-34-0) to afford after workup and purification using method-A, methyl 5-bromo-1-(tetrahydro-2H-pyran-4-yl)-1H-indazole-3-carboxylate (281b) (6.354 g, 47.0% yield) as a white solid; ¹H NMR (300 MHz, DMSO-d₆) δ 7.97 (d, J=9.0 Hz, 1H), 7.69-7.63 (m, 2H), 5.07 (td, J=11.3, 5.4 Hz, 1H), 4.07-3.98 (m, 2H), 3.93 (s, 3H), 3.57 (td, J=11.8, 2.1 Hz, 2H), 2.09 (qd, J=12.1, 4.5 Hz, 2H), 2.03-1.83 (m, 2H); MS (ES+): 339.00 (M+1) and methyl 5-bromo-2-(tetrahydro-2H-pyran-4-yl)-2H-indazole-3-carboxylate (281c) (2.135 g, 6.29 mmol, 15.79% yield) as a white solid; MS (ES+): 339.10 (M+1).

### Step-2: Preparation of (5-bromo-1-(tetrahydro-2H-pyran-4-yl)-1H-indazol-3-yl)methanol (281d)

Compound 281d was prepared according to the procedure reported in step-2 of scheme-119 from methyl 5-bromo-1-(tetrahydro-2H-pyran-4-yl)-1H-indazole-3-carboxylate (281b) (6.354 g, 18.73 mmol) in THF (150 mL) using LiBH₄ (23.42 mL, 46.8 mmol) and MeOH (1.895 mL, 46.8 mmol) to afford after workup and purification using method-AW, (5-bromo-1-(tetrahydro-2H-pyran-4-yl)-1H-indazol-3-yl)methanol (281d) (3.216 g, 55.2% yield) as a clear oil; ¹H NMR (300 MHz, DMSO-d₆) δ 8.06 (d, J=1.9 Hz, 1H), 7.73 (d, J=9.0 Hz, 1H), 7.50 (dd, J=8.9, 1.9 Hz, 1H), 5.34 (t, J=5.9 Hz, 1H), 4.93-4.78 (m, 1H), 4.75 (d, J=5.9 Hz, 2H), 4.05-3.94 (m, 2H), 3.54 (td, J=12.0, 2.0 Hz, 2H), 2.18-1.96 (m, 2H), 1.86 (dd, 2H); MS (ES+): 311.00 (M+1).

### Step-3: Preparation of ethyl 2-(2-((5-bromo-1-(tetrahydro-2H-pyran-4-yl)-1H-indazol-3-yl)methoxy)phenyl)acetate (281e)

Compound 281e was prepared according to the procedure reported in step-2 of scheme-2 from (5-bromo-1-(tetrahydro-2H-pyran-4-yl)-1H-indazol-3-yl)methanol (281d) (3.216 g, 10.34 mmol) in DCM (10 mL) using PPh₃ (2.98 g, 11.37 mmol), ethyl 2-(2-hydroxyphenyl)acetate (2b) (2.235 g, 12.40 mmol) and a solution of DCAD (4.17 g, 11.37 mmol) in DCM (10 mL) to afford after workup and purification using method-AU, ethyl 2-(2-((5-bromo-1-(tetrahydro-2H-pyran-4-yl)-1H-indazol-3-yl)methoxy)phenyl)acetate (281e) (3.104 g, 63.4% yield) as a white solid; MS (ES+): 473.1 and 475.1 (M+1); (ES−): 471.1 and 473.0 (M−1).

### Step-4: Preparation of ethyl 2-(2-((5-(1-aminoisoquinolin-7-yl)-1-(tetrahydro-2H-pyran-4-yl)-1H-indazol-3-yl)methoxy)phenyl)acetate (281f)

Compound 281f was prepared according to the procedure reported in step-5 of scheme-1 from ethyl 2-(2-((5-bromo-1-(tetrahydro-2H-pyran-4-yl)-1H-indazol-3-yl)methoxy)phenyl)acetate (281e) (700 mg, 1.479 mmol) in dioxane/MeTHF (10 mL, 1:1) using 1-aminoisoquinolin-7-ylboronic acid (87a) (361 mg, 1.922 mmol), K₃PO₄ (2 M aqueous solution, 1.479 mL, 5.92 mmol), PCy₃ (83 mg, 0.296 mmol), Pd₂(dba)₃ (135 mg, 0.148 mmol) and PdCl₂(dppf)-CH₂Cl₂ adduct (121 mg, 0.148 mmol) to afford after workup and purification using method-AE, ethyl 2-(2-((5-(1-aminoisoquinolin-7-yl)-1-(tetrahydro-2H-pyran-4-yl)-1H-indazol-3-yl)methoxy)phenyl)acetate (281f) (331 mg, 41.7% yield) as a yellow solid; MS (ES+): 537.30 (M+1).

### Step-5: Preparation of 2-(2-((5-(1-aminoisoquinolin-7-yl)-1-(tetrahydro-2H-pyran-4-yl)-1H-indazol-3-yl)methoxy)phenyl)acetic acid (281g)

Compound 281g was prepared according to the procedure reported in step-2 of scheme-1 from ethyl 2-(2-((5-(1-aminoisoquinolin-7-yl)-1-(tetrahydro-2H-pyran-4-yl)-1H-indazol-3-yl)methoxy)phenyl)acetate (2811) (331 mg, 0.617 mmol) in THF (3 mL) using a solution of LiOH·H₂O (78 mg, 1.850 mmol) in water (1 mL) in water (1 mL) to afford after workup and purification using method-M, 2-(2-((5-(1-aminoisoquinolin-7-yl)-1-(tetrahydro-2H-pyran-4-yl)-1H-indazol-3-yl)methoxy)phenyl)acetic acid (281g) (80.1 mg, 25.5% yield) HCl salt as a white solid; ¹H NMR (300 MHz, DMSO-d₆) δ 13.54 (s, 1H, D₂O exchangeable), 12.18 (s, 1H, D₂O exchangeable), 9.20 (s, 2H, D₂O exchangeable), 9.01 (s, 1H), 8.47-8.38 (m, 1H), 8.33 (s, 1H), 8.04 (d, J=8.6 Hz, 2H), 8.01-7.93 (m, 1H), 7.70 (d, J=6.9 Hz, 1H), 7.33-7.24 (m, 3H), 7.21 (d, J=7.3 Hz, 1H), 6.98-6.88 (m, 1H), 5.49 (s, 2H), 5.08-4.92 (m, 1H), 4.10-3.97 (m, 2H), 3.66-3.57 (m, 2H), 3.54 (s, 2H), 2.25-2.05 (m, 2H), 2.03-1.85 (m, 2H); MS (ES+): 509.10 (M+1); (ES−): 507.10 (M−1).

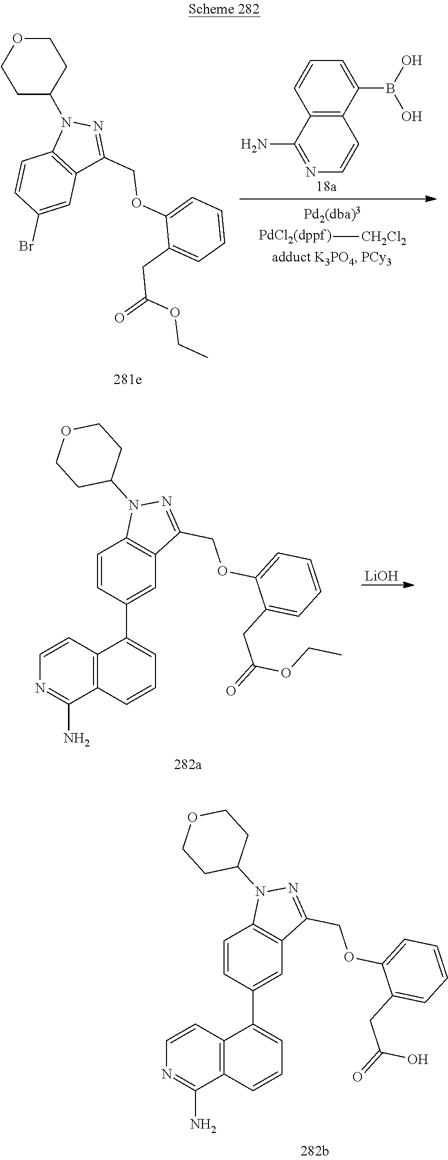

### Preparation of 2-(2-((5-(1-aminoisoquinolin-5-yl)-1-(tetrahydro-2H-pyran-4-yl)-1H-indazol-3-yl)methoxy)phenyl)acetic acid (282b)

### Step-1: Preparation of ethyl 2-(2-((5-(1-aminoisoquinolin-5-yl)-1-(tetrahydro-2H-pyran-4-yl)-1H-indazol-3-yl)methoxy)phenyl)acetate (282a)

Compound 282a was prepared according to the procedure reported in step-5 of scheme-1 from ethyl 2-(2-((5-bromo-1-(tetrahydro-2H-pyran-4-yl)-1H-indazol-3-yl)methoxy)phenyl)acetate (281e) (700 mg, 1.479 mmol) in dioxane/MeTHF (10 mL, 1:1) using (1-aminoisoquinolin-5-yl)boronic acid (18a) (361 mg, 1.922 mmol), K₃PO₄ (2 M aqueous solution, 1.479 mL, 5.92 mmol), PCy₃ (83 mg, 0.296 mmol), Pd₂(dba)₃ (135 mg, 0.148 mmol) and PdCl₂(dppf)-CH₂Cl₂ adduct (121 mg, 0.148 mmol) to afford after workup and purification using method-AE, ethyl 2-(2-((5-(1-aminoisoquinolin-5-yl)-1-(tetrahydro-2H-pyran-4-yl)-1H-indazol-3-yl)methoxy)phenyl)acetate (282a) (168 mg, 21.17% yield) as a yellow solid. MS (ES+): 537.20 (M+1).

### Step-2: Preparation of 2-(2-((5-(1-aminoisoquinolin-5-yl)-1-(tetrahydro-2H-pyran-4-yl)-1H-indazol-3-yl)methoxy)phenyl)acetic acid (282b)

Compound 282b was prepared according to the procedure reported in step-2 of scheme-1 from ethyl 2-(2-((5-(1-aminoisoquinolin-5-yl)-1-(tetrahydro-2H-pyran-4-yl)-1H-indazol-3-yl)methoxy)phenyl)acetate (282a) (168 mg, 0.313 mmol) in THF (3 mL) using a solution of LiOH·H₂O (39.4 mg, 0.939 mmol) in water (1 mL) to afford after workup and purification using method-M, 2-(2-((5-(1-aminoisoquinolin-5-yl)-1-(tetrahydro-2H-pyran-4-yl)-1H-indazol-3-yl)methoxy)phenyl)acetic acid (282b) (118.1 mg, 0.232 mmol, 74.2% yield) HCl salt as a white solid; ¹H NMR (300 MHz, DMSO-d₆) δ 13.30 (s, 1H, D₂O exchangeable), 12.05 (s, 1H, D₂O exchangeable), 9.14 (s, 2H, D₂O exchangeable), 8.62 (d, J=8.3 Hz, 1H), 8.00-7.93 (m, 2H), 7.91-7.81 (m, 2H), 7.63 (d, J=7.3 Hz, 1H), 7.49 (dd, J=8.6, 1.6 Hz, 1H), 7.30-7.22 (m, 2H), 7.18 (d, J=7.3 Hz, 1H), 7.00 (d, J=7.3 Hz, 1H), 6.95-6.87 (m, 1H), 5.46 (s, 2H), 5.08-4.92 (m, 1H), 4.12-3.99 (m, 2H), 3.66-3.56 (m, 2H), 3.49 (s, 2H), 2.30-2.08 (m, 2H), 2.05-1.87 (m, 2H); MS (ES+): 509.20 (M+1); (ES−): 507.20 (M−1).

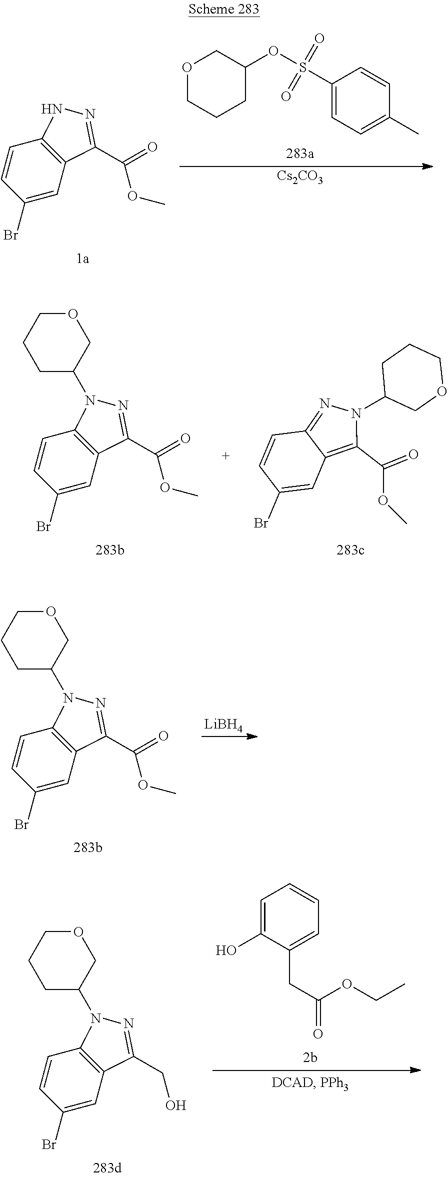

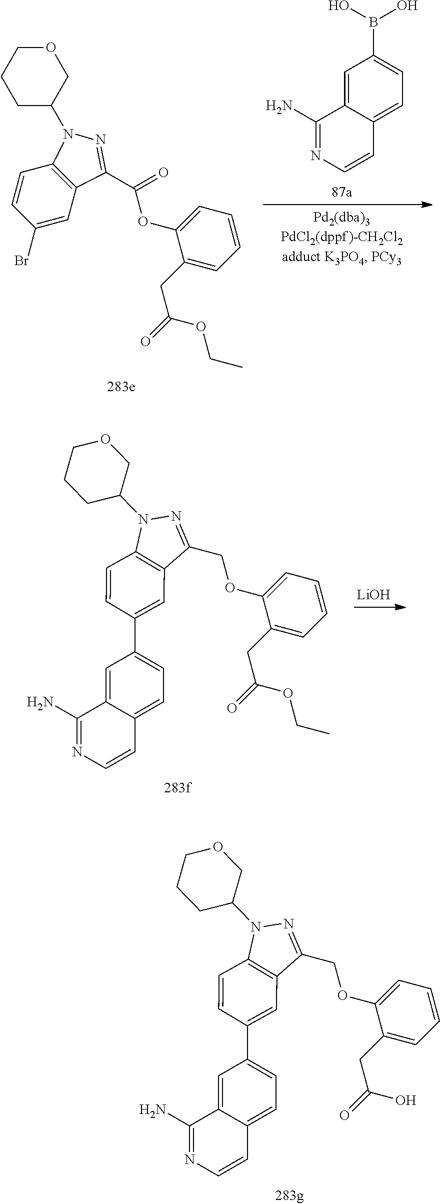

### Preparation of 2-(2-((5-(1-aminoisoquinolin-7-yl)-1-(tetrahydro-2H-pyran-3-yl)-1H-indazol-3-yl)methoxy)phenyl)acetic acid (283g)

### Step-1: Preparation of methyl 5-bromo-1-(tetrahydro-2H-pyran-3-yl)-1H-indazole-3-carboxylate (283b) and methyl 5-bromo-2-(tetrahydro-2H-pyran-3-yl)-2H-indazole-3-carboxylate (283c)

Compounds 283b and 283c was prepared according to the procedure reported in step-2 of scheme-86 from methyl 5-bromo-1H-indazole-3-carboxylate (1a) (22.39 g, 88 mmol) in DMF (60 mL) using Cs₂CO₃ (57.2 g, 176 mmol) and tetrahydro-2H-pyran-3-yl 4-methylbenzenesulfonate (22.5 g, 88 mmol) to afford after workup and purification using method-A, methyl 5-bromo-1-(tetrahydro-2H-pyran-3-yl)-1H-indazole-3-carboxylate (283b) (3.233 g, 10.86% yield) as a white solid; ¹H NMR (300 MHz, DMSO-d₆) δ 8.21 (d, J=1.8 Hz, 1H), 7.98 (d, J=9.0 Hz, 1H), 7.64 (dd, J=9.0, 1.9 Hz, 1H), 5.02-4.86 (m, 1H), 4.01 (dd, J=10.7, 4.3 Hz, 1H), 3.93 (s, 3H), 3.89 (t, J=3.3 Hz, 1H), 3.67 (t, J=10.4 Hz, 1H), 3.50-3.44 (m, 1H), 2.17 (q, J=8.2, 7.4 Hz, 2H), 1.84 (dd, J=7.8, 4.3 Hz, 2H); MS (ES+): 339.00 and 341.1 (M+1) and methyl 5-bromo-2-(tetrahydro-2H-pyran-3-yl)-2H-indazole-3-carboxylate (283c) as a white solid; MS (ES+): 339.00 and 341.0 (M+1).

### Step-2: Preparation of (5-bromo-1-(tetrahydro-2H-pyran-3-yl)-1H-indazol-3-yl)methanol (283d)

Compound 283d was prepared according to the procedure reported in step-2 of scheme-119 from methyl 5-bromo-1-(tetrahydro-2H-pyran-3-yl)-1H-indazole-3-carboxylate (283b) (3.233 g, 9.53 mmol) in THF (75 mL) using LiBH₄ (11.91 mL, 23.83 mmol) and MeOH (0.964 mL, 23.83 mmol) to afford after workup and purification using method-AW. (5-bromo-1-(tetrahydro-2H-pyran-3-yl)-1H-indazol-3-yl)methanol (283d) (2.408 g, 81% yield) as a clear oil. ¹H NMR (300 MHz, DMSO-d₆) δ 8.05 (d, J=1.8 Hz, 1H), 7.74 (d, J=9.0 Hz, 1H), 7.49 (dd. J=8.9, 1.9 Hz, 1H), 5.35 (t, J=5.9 Hz, 1H), 4.72 (dd. J=11.8, 5.4 Hz, 3H), 3.92 (ddd, J=11.9, 8.2, 4.3 Hz, 2H), 3.63 (t, J=10.5 Hz, 1H), 3.48-3.33 (m, 1H), 2.13 (dd. J=19.3, 7.7 Hz, 2H), 1.79 (dp, J=10.3, 4.3 Hz, 2H); MS (ES+): 311.00 (M+1).

### Step-3: Preparation of ethyl 2-(2-((5-bromo-1-(tetrahydro-2H-pyran-3-yl)-1H-indazol-3-yl)methoxy)phenyl)acetate (283e)

Compound 283e was prepared according to the procedure reported in step-2 of scheme-2 from (5-bromo-1-(tetrahydro-2H-pyran-3-yl)-1H-indazol-3-yl)methanol (283d) (2.408 g, 7.74 mmol) in DCM (10 mL) using PPh₃ (2.233 g, 8.51 mmol), ethyl 2-(2-hydroxyphenyl)acetate (2b) (1.673 g, 9.29 mmol) and a solution of DCAD (3.13 g, 8.51 mmol) in DCM (10 mL) to afford after workup and purification using method-AU, ethyl 2-(2-((5-bromo-1-(tetrahydro-2H-pyran-3-yl)-1H-indazol-3-yl)methoxy)phenyl)acetate (283e) (2.002 g, 54.7% yield) as a white solid; MS (ES+): 473.10 (M+1).

### Step-4: Preparation of ethyl 2-(2-((5-(1-aminoisoquinolin-7-yl)-1-(tetrahydro-2H-pyran-3-yl)-1H-indazol-3-yl)methoxy)phenyl)acetate (283f)

Compound 283f was prepared according to the procedure reported in step-5 of scheme-1 from ethyl 2-(2-((5-bromo-1-(tetrahydro-2H-pyran-3-yl)-1H-indazol-3-yl)methoxy)phenyl)acetate (283e) (700 mg, 1.479 mmol) in dioxane/MeTHF (10 mL, 1:1) using 1-aminoisoquinolin-7-ylboronic acid (87a) (361 mg, 1.922 mmol), K₃PO₄ (2 M aqueous solution, 1.479 mL, 5.92 mmol). PCy₃ (83 mg, 0.296 mmol), Pd₂(dba)₃ (135 mg, 0.148 mmol) and PdCl₂(dppf)-CH₂Cl₂ adduct (121 mg, 0.148 mmol) to afford after workup and purification using method-AE, ethyl 2-(2-((5-(1-aminoisoquinolin-7-yl)-1-(tetrahydro-2H-pyran-3-yl)-1H-indazol-3-yl)methoxy)phenyl)acetate (283f) (398 mg, 50.2% yield) as a yellow solid. MS (ES+): 537.30 (M+1).

### Step-5: Preparation of 2-(2-((5-(1-aminoisoquinolin-7-yl)-1-(tetrahydro-2H-pyran-3-yl)-1H-indazol-3-yl)methoxy)phenyl)acetic acid (283g)

Compound 283g was prepared according to the procedure reported in step-2 of scheme-1 from ethyl2-(2-((5-(1-aminoisoquinolin-7-yl)-1-(tetrahydro-2H-pyran-3-yl)-1H-indazol-3-yl)methoxy)phenyl)acetate (283f) (398 mg, 0.742 mmol) in THF (3 mL) using a solution of LiOH·H₂O (93 mg, 2.225 mmol) in water (1 mL) in water (1 mL) to afford after workup and purification using method-M, 2-(2-((5-(1-aminoisoquinolin-7-yl)-1-(tetrahydro-2H-pyran-3-yl)-1H-indazol-3-yl)methoxy)phenyl)acetic acid (283g) (83.9 mg, 22.24% yield) HCl salt as a white solid; ¹H NMR (300 MHz, DMSO-d₆) δ 13.24 (s, 1H, D₂O exchangeable), 12.09 (s, 1H, D₂O exchangeable), 9.13 (s, 2H, D₂O exchangeable), 8.95 (s, 1H), 8.42 (dd, J=8.4, 1.6 Hz, 1H), 8.31 (s, 1H), 8.04 (d, J=8.5 Hz, 1H), 8.01 (s, 2H), 7.69 (d, J=6.9 Hz, 1H), 7.33-7.23 (m, 3H), 7.20 (d, J=7.4 Hz, 1H), 6.97-6.87 (m, 1H), 5.47 (s, 2H), 4.94-4.78 (m, 1H), 4.07-3.87 (m, 2H), 3.72 (t, J=10.5 Hz, 1H), 3.53 (s, 2H), 3.51-3.41 (m, 1H), 2.31-2.10 (m, 2H), 1.93-1.76 (m, 2H); MS (ES+): 509.20 (M+1); (ES−): 507.10 (M−1).

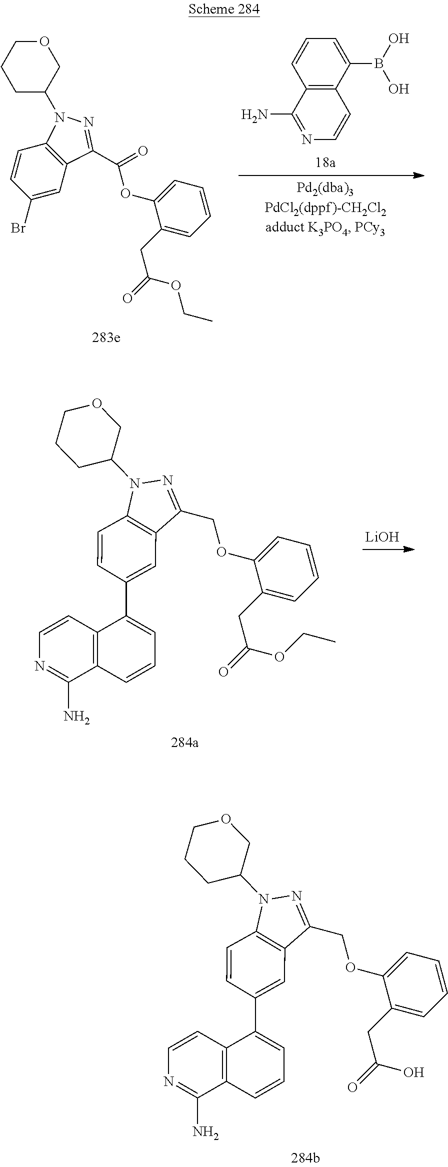

### Preparation of 2-(2-((5-(1-aminoisoquinolin-5-yl)-1-(tetrahydro-2H-pyran-3-yl)-1H-indazol-3-yl)methoxy)phenyl)acetic acid (284b)

### Step-1: Preparation of ethyl 2-(2-((5-(1-aminoisoquinolin-5-yl)-1-(tetrahydro-2H-pyran-3-yl)-1H-indazol-3-yl)methoxy)phenyl)acetate (284a)

Compound 284a was prepared according to the procedure reported in step-5 of scheme-1 from ethyl 2-(2-((5-bromo-1-(tetrahydro-2H-pyran-3-yl)-1H-indazol-3-yl)methoxy)phenyl)acetate (283e) (700 mg, 1.479 mmol) in dioxane/MeTHF (10 mL, 1:1) using (I-aminoisoquinolin-5-yl)boronic acid (18a) (361 mg, 1.922 mmol), K₃PO₄ (2 M aqueous solution, 1.479 mL, 5.92 mmol), PCy₃ (83 mg, 0.296 mmol), Pd₂(dba)₃ (135 mg, 0.148 mmol) and PdCl₂(dppf)-CH₂Cl₂ adduct (121 mg, 0.148 mmol) to afford after workup and purification using method-AE, ethyl 2-(2-((5-(1-aminoisoquinolin-5-yl)-1-(tetrahydro-2H-pyran-3-yl)-1H-indazol-3-yl)methoxy)phenyl)acetate (284a) (120 mg, 15.12% yield) as a yellow solid; MS (ES+): 537.30 (M+1).

### Step-2: Preparation of 2-(2-((5-(1-aminoisoquinolin-5-yl)-1-(tetrahydro-2H-pyran-3-yl)-1H-indazol-3-yl)methoxy)phenyl)acetic acid (284b)

Compound 284b was prepared according to the procedure reported in step-2 of scheme-1 from ethyl 2-(2-((5-(1-aminoisoquinolin-5-yl)-1-(tetrahydro-2H-pyran-3-yl)-1H-indazol-3-yl)methoxy)phenyl)acetate (284a) (120 mg, 0.224 mmol) in THF (3 mL) using a solution of LiOH·H₂O (28.2 mg, 0.671 mmol) in water (1 mL) in water (1 mL) to afford after workup and purification using method-M, 2-(2-((5-(1-aminoisoquinolin-5-yl)-1-(tetrahydro-2H-pyran-3-yl)-1H-indazol-3-yl)methoxy)phenyl)acetic acid (284b) (144.4 mg, 127% yield) HCl salt as a white solid; ¹H NMR (300 MHz, DMSO-d₆) $13.37 (s, 1H, D₂O Exchangeable), 12.03 (s, 1H, D₂O Exchangeable), 9.20 (s, 2H, D₂O Exchangeable), 8.63 (d, J=8.3 Hz, 1H), 8.01-7.91 (m, 2H), 7.91-7.77 (m, 2H), 7.62 (d, J=7.3 Hz, 1H), 7.48 (dd, J=8.7, 1.6 Hz, 1H), 7.24 (d, J=4.1 Hz, 2H), 7.17 (d, J=7.4 Hz, 1H), 6.98 (d, J=7.2 Hz, 1H), 6.94-6.84 (m, 1H), 5.44 (s, 2H), 4.93-4.76 (m, 1H), 4.07-3.99 (m, 1H), 3.97-3.87 (m, 1H), 3.75 (t, J=10.5 Hz, 1H), 3.53-3.43 (m, 3H), 2.33-2.11 (m, 2H), 1.94-1.74 (m, 2H); MS (ES+): 509.10 (M+1); (ES−): 507.10 (M−1).

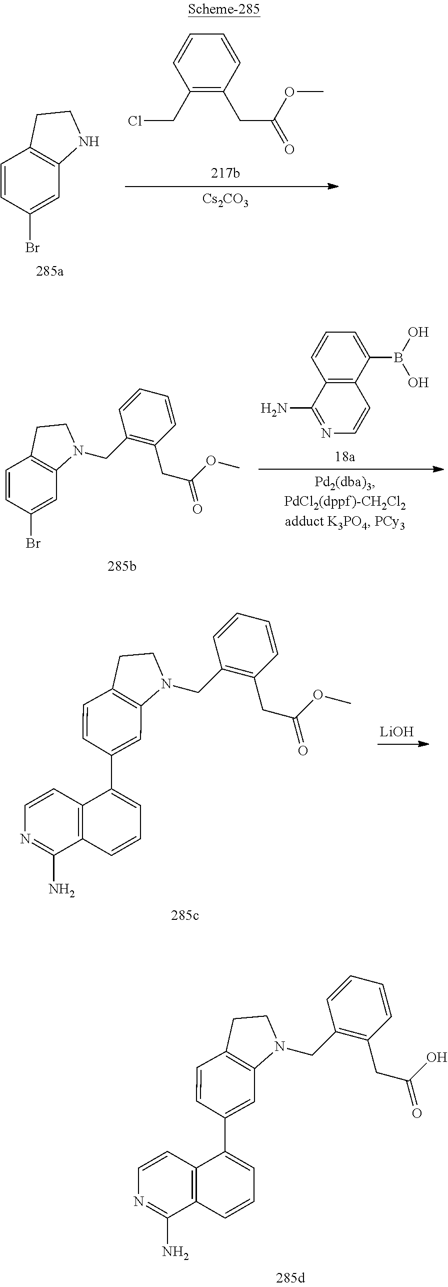

### Preparation of 2-(2-((6-(1-aminoisoquinolin-5-yl)indolin-1-yl)methyl)phenyl)acetic acid (285d)

### Step-1: Preparation of methyl 2-(2-((6-bromoindolin-1-yl)methyl)phenyl)acetate (285b)

To a solution of 6-bromoindoline (285a) (200 mg, 1.01 mmol) in DMF (10 mL) at 0° C. was added cesium carbonate (987 mg, 3.03 mmol) stirred for 15 min and added methyl 2-(2-(chloromethyl)phenyl)acetate (217b) (221 mg, 1.111 mmol) at 0° C. The reaction was warmed to 60° C., stirred for 15 h, poured into EtOAc and washed with water and brine. The organic layer was dried, filtered and concentrated in vacuo. The residue obtained was purified using method-J, to give methyl 2-(2-((6-bromoindolin-1-yl)methyl)phenyl)acetate (285b) as a white solid; MS (ES+): 360.00 & 362.00 (M+1).

### Step-2: Preparation of methyl 2-(2-((6-(1-aminoisoquinolin-5-yl)indolin-1-vi)methyl)phenyl)acetate (285c)

Prepared according to the procedure reported in step-5 of scheme-1, from methyl 2-(2-((6-bromoindolin-1-yl)methyl)phenyl)acetate (285b) (200 mg, 0.555 mmol) in dioxane/THF (6 mL; ratio 1:1) using (1-aminoisoquinolin-5-yl)boronic acid (18a) (157 mg, 0.833 mmol), 2 M aqueous solution of tripotassium phosphate (1.11 mL, 2.221 mmol), tricyclohexylphosphine (31.1 mg, 0.111 mmol), PdCl₂(dppf)-CH₂Cl₂Adduct (45.3 mg, 0.056 mmol) and Pd₂(dba)₃ (50.8 mg, 0.056 mmol) and heating at 90° C. for 2 h. This gave after work up and purification using method-T, methyl 2-(2-((6-(1-aminoisoquinolin-5-yl)indolin-1-yl)methyl)phenyl)acetate (285c) (120 mg, 57.3% yield) as a clear oil; MS (ES+): 424.20 (M+1).

### Step-3: Preparation of 2-(2-((6-(1-aminoisoquinolin-5-yl)indolin-1-yl)methyl)phenyl)acetic acid (285d)

Prepared according to the procedure reported in step-2 of scheme-1, from methyl 2-(2-((6-(1-aminoisoquinolin-5-yl)indolin-1-yl)methyl)phenyl)acetate (285c) (150 mg, 0.354 mmol) in MeOH/THF (6 mL) using lithium hydroxide hydrate (89 mg, 2.125 mmol) in water (2 mL) to afford after work up and purification using method-G, 2-(2-((6-(1-aminoisoquinolin-5-yl)indolin-1-yl)methyl)phenyl)acetic acid (285d) (35 mg, 24% yield) HCl salt as a white solid; ¹H NMR (300 MHz, DMSO-d₆) δ 13.72-13.31 (m, 1H, DO exchangeable), 9.26 (s, 2H, D₂O exchangeable), 8.61 (d, J=8.0 Hz, 1H), 7.89-7.73 (m, 2H), 7.66-7.55 (m, 1H), 7.41-7.30 (m, 1H), 7.30-7.14 (m, 4H), 7.01 (d, J=7.2 Hz, 1H), 6.63 (dd, 1H), 6.57-6.46 (m, 1H), 4.28 (s, 2H), 3.68 (s, 2H), 3.30 (t, J=8.3 Hz, 2H), 2.98 (t, J=8.3 Hz, 2H); MS (ES+): 410.10 (M+1); (ES−): 408.10 (M−1).

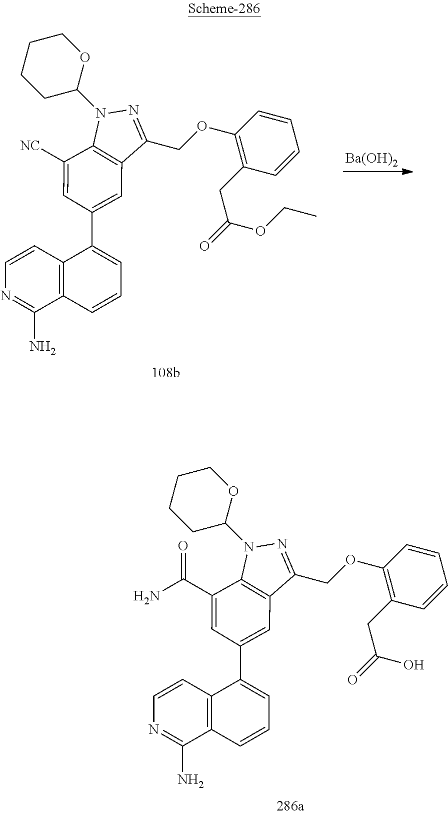

### Preparation of 2-(2-((5-(1-aminoisoquinolin-5-yl)-7-carbamoyl-1-(tetrahydro-2H-pyran-2-yl)-1H-indazol-3-yl)methoxy)phenyl)acetic acid (286a)

To a solution of ethyl 2-(2-((5-(1-aminoisoquinolin-5-yl)-7-cyano-1-(tetrahydro-2H-pyran-2-yl)-1H-indazol-3-yl)methoxy)phenyl)acetate (108b) (130 mg, 0.231 mmol) in 2-propanol (4 mL) and water (12 mL) was added barium hydroxide (198 mg, 1.157 mmol) and heated at 100° C. for 24 h. The reaction was cooled to RT, acidified with sat. aq. KHSO₄, and the resulting mixture was stirred for 30 min. The reaction mixture was extracted with EtOAc, washed with water, brine, dried, filtered and concentrated in vacuo. The residue obtained was purified using method-G to afford 2-(2-((5-(1-aminoisoquinolin-5-yl)-7-carbamoyl-1-(tetrahydro-2H-pyran-2-yl)-1H-indazol-3-yl)methoxy)phenyl)acetic acid (286a) (30 mg, 24% yield) HCl salt as a white solid; ¹H NMR (300 MHz, DMSO-d₆) δ 13.50-12.97 (m, 1H, D₂O exchangeable), 12.01 (s, 1H, D₂O exchangeable), 9.14 (s, 2H, D₂O exchangeable), 8.63 (d, J=8.3 Hz, 1H), 8.18 (s, 1H, D₂O exchangeable), 8.12-7.95 (m, 2H), 7.87 (t, J=7.9 Hz, 1H), 7.75 (s, 1H, D₂O exchangeable), 7.62 (d, J=7.2 Hz, 1H), 7.57 (d, 1H), 7.31-7.21 (m, 2H), 7.18 (d, J=7.4 Hz, 1H), 7.11 (d, J=7.3 Hz, 1H), 6.97-6.82 (m, 1H), 6.15-6.04 (m, 1H), 5.47 (s, 2H), 3.82-3.70 (m, 1H), 3.67-3.53 (m, 1H), 3.48 (s, 2H), 2.46-2.31 (m, 1H), 2.31-2.13 (m, 1H), 2.13-1.99 (m, 1H), 1.77-1.44 (m, 3H); MS (ES+): 552.20 (M+1); (ES−): 550.10 (M−1).

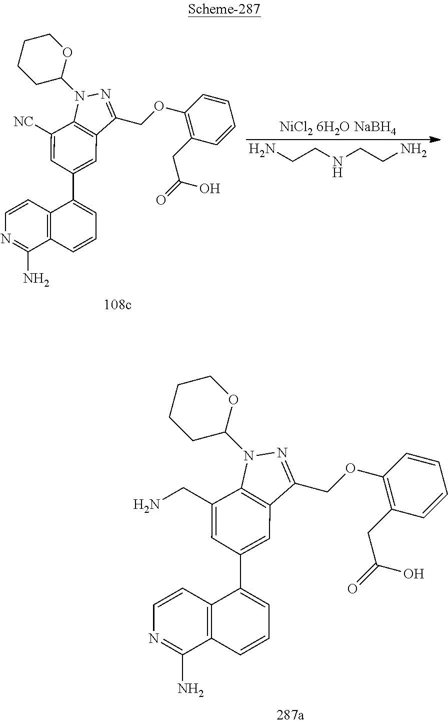

### Preparation of 2-(2-((5-(1-aminoisoquinolin-5-yl)-7-(aminomethyl)-1-(tetrahydro-2H-pyran-2-yl)-1H-indazol-3-yl)methoxy)phenyl)acetic acid (287a)

To a solution of 2-(2-((5-(1-aminoisoquinolin-5-yl)-7-cyano-1-(tetrahydro-2H-pyran-2-yl)-1H-indazol-3-yl)methoxy)phenyl)acetic acid (108c) (120 mg, 0.225 mmol) in anhydrous ethanol (10 mL), cooled to 0° C., was added nickel(II) chloride hexahydrate (13.36 mg, 0.056 mmol) followed by sodium borohydride (25.5 mg, 0.675 mmol) in small portions over a period of 10 min (caution: reaction is exothermic and effervescent) and stirred at room temperature for 24 h. Once the reaction was complete N1-(2-aminoethyl)ethane-1,2-diamine (0.049 mL, 0.450 mmol) was added stirred for 1 h and concentrated in vacuum. The residue obtained was treated with brine, extracted with ethyl acetate (2×150 mL), dried filtered and concentrated in vacuum. The residue obtained was purified using method-BI to furnish crude product, which was further purified using method-G to give 2-(2-((5-(1-aminoisoquinolin-5-yl)-7-(aminomethyl)-1-(tetrahydro-2H-pyran-2-yl)-1H-indazol-3-yl)methoxy)phenyl)acetic acid (287a) (12 mg, 10% yield) HCl salt as a white solid; ¹H NMR (300 MHz, DMSO-d₆) δ 13.42 (s, 1H, D₂O exchangeable), 12.03 (s, 1H, D₂O exchangeable), 9.28 (s, 2H, D₂O exchangeable), 8.78-8.58 (m, 4H, 3H D₂O exchangeable), 8.29 (s, 1H, D₂O exchangeable), 8.06-7.95 (m, 2H), 7.89 (t, J=7.8 Hz, 1H), 7.69-7.58 (m, 2H), 7.25 (d, J=4.1 Hz, 2H), 7.18 (d, J=7.4 Hz, 1H), 7.09 (d, J=7.2 Hz, 1H), 6.97-6.84 (m, 1H), 5.97-5.84 (m, 1H), 5.46 (s, 2H), 4.68-4.37 (m, 2H), 3.93-3.75 (m, 2H), 3.47 (s, 2H), 2.59-2.53 (m, 1H), 2.25-2.05 (m, 2H), 1.85-1.71 (m, 1H), 1.69-1.52 (m, 2H); MS (ES+): 538.20 (M+1); MS (ES−): 536.20 (M−1).

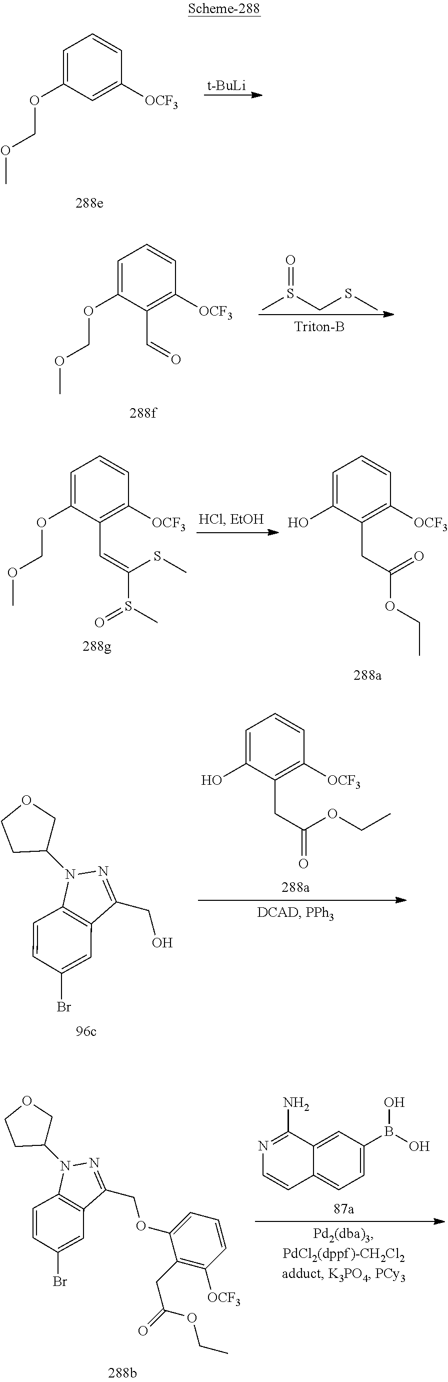

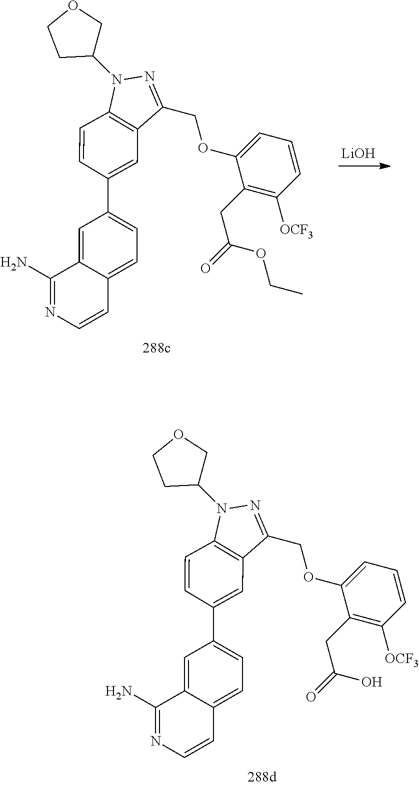

### Preparation of 2-(2-((5-(1-aminoisoquinolin-7-yl)-1-(tetrahydrofuran-3-yl)-1H-indazol-3-yl)methoxy)-6-(trifluoromethoxy)phenyl)acetic acid (288d)

### Step-1: Preparation of 2-(methoxymethoxy)-6-(trifluoromethoxy)benzaldehyde (288f)

To a solution of 1-(methoxymethoxy)-3-(trifluoromethoxy)benzene (288f) (25.0 g, 112.53 mmol; CAS #851341-38-3) in THF (200 mL) at −78° C. was slowly added tert-butyl lithium in pentane (1.5 M, 75.0 mL, 112.02 mmol) and stirred at −78° C. for 2 h. To this DMF (27 mL) was added at −78° C. and was stirred at −78° C. for 1 h. Reaction mixture was slowly quenched with saturated NH₄Cl solution (500 mL) and extracted with ethyl acetate (3×500 mL), dried, filtered and concentrated in vacuo. The residue obtained was purified using column chromatography [silica gel, eluting with 20% EtOAc in n-heptane] to give 2-(methoxymethoxy)-6-(trifluoromethoxy)benzaldehyde (288f) (28.02 g, 99.5% yield) as a colorless liquid; ¹H NMR (300 MHz, DMSO-d₆) δ 10.40 (s, 1H), 7.83-7.61 (m, 1H), 7.48-7.28 (m, 1H), 7.21-7.04 (m, 1H), 5.39 (d, J=0.8 Hz, 2H), 3.45 (d, J=0.8 Hz, 3H).

### Step-2: Preparation of (E)-(2-(2-(methoxymethoxy)-6-(trifluoromethoxy)phenyl)-1-(methylsulfinyl)vinyl)(methyl)sulfane (288g)

Compound 288g was prepared according to the procedure reported in step-2 of scheme-267, from 2-(methoxymethoxy)-6-(trifluoromethoxy)benzaldehyde (288f) (28.0 g, 111.92 mmol) in THF (200 mL) using methyl((methylsulfinyl)methyl)sulfane (22.24 g, 179.07 mmol) 25% Triton-B in MeOH (44.93 g, 67.152 mmol) and heating at 66° C. overnight to afford after workup and purification using column chromatography [silica gel, eluting with 40% EtOAc in n-heptane] (E)-(2-(2-(methoxymethoxy)-6-(trifluoromethoxy)phenyl)-1-(methylsulfinyl)vinyl)(methyl)sulfane (288g) (26.2 g, 66% yield) as a colorless liquid; 1H NMR (300 MHz, DMSO-d₆) δ 7.48 (td, J=8.4, 0.7 Hz, 1H), 7.34 (s, 1H), 7.23 (dd, J=8.5, 0.9 Hz, 1H), 7.10 (dt, J=8.3, 1.3 Hz, 1H), 5.28 (s, 2H), 3.39 (s, 3H), 2.77 (s, 3H), 2.20 (s, 3H).

### Step-3: Preparation of ethyl 2-(2-hydroxy-6-(trifluoromethoxy)phenyl)acetate (288a)

Compound 288a was prepared according to the procedure reported in step-3 of scheme-267, from (E)-(2-(2-(methoxymethoxy)-6-(trifluoromethoxy)phenyl)-1-(methylsulfinyl)vinyl)(methyl)sulfane (288g) (26.2 g, 73.52 mmol) in ethanol (131 mL) using HCl in EtOH (131 mL) and heating at reflux for 1 h to afford after workup and purification using column chromatography [silica gel, eluting with 20% EtOAc in n-heptane] ethyl 2-(2-hydroxy-6-(trifluoromethoxy)phenyl)acetate (288a) (14.8 g, 76% yield) as a light yellow solid; H NMR (300 MHz, DMSO-d₆) δ 10.20 (s, 1H), 7.21 (t, J=8.3 Hz, 1H), 6.85 (dd, J=8.3, 1.0 Hz, 1H), 6.82-6.73 (m, 1H), 4.07 (q, J=7.1 Hz, 2H), 3.59 (s, 2H), 1.16 (t, J=7.1 Hz, 3H); MS (ES−): 263.1 (M−1).

### Step-4: Preparation of ethyl 2-(2-((5-bromo-1-(tetrahydrofuran-3-yl)-1H-indazol-3-yl)methoxy)-6-(trifluoromethoxy)phenyl)acetate (288b)

Prepared according to the procedure reported in step-2 of scheme-2, from (5-bromo-1-(tetrahydrofuran-3-yl)-1H-indazol-3-yl)methanol (96c) (2.0 g, 6.73 mmol) in DCM (50 mL) using ethyl 2-(2-hydroxy-6-(trifluoromethoxy)phenyl)acetate (288a) (2.134 g, 8.08 mmol), PPh₃ (1.942 g, 7.40 mmol) a solution of DCAD (2.72 g, 7.40 mmol) in DCM (15 mL) to afford after workup and purification using method-AU, ethyl 2-(2-((5-bromo-1-(tetrahydrofuran-3-yl)-1H-indazol-3-yl)methoxy)-6-(trifluoromethoxy)phenyl) acetate (288b) (1.47 g, 40% yield) as a white solid; MS (ES+): 543.10 (M+1); (ES−): 541.00 (M−1).

### Step-5: Preparation of ethyl 2-(2-((5-(1-aminoisoquinolin-7-yl)-1-(tetrahydrofuran-3-yl)-1H-indazol-3-yl)methoxy)-6-(trifluoromethoxy)phenyl)acetate (288c)

Prepared according to the procedure reported in step-2 of scheme-18, from ethyl 2-(2-((5-bromo-1-(tetrahydrofuran-3-yl)-1H-indazol-3-yl)methoxy)-6-(trifluoromethoxy)phenyl)acetate (288b) (700 mg, 1.288 mmol) in dioxane/MeTHF (10 mL, Ratio: 1:1) using (1-aminoisoquinolin-7-yl)boronic acid (87a) (315 mg, 1.675 mmol), aqueous tripotassium phosphate (2M, 1288 μl, 5.15 mmol), tricyclohexylphosphine (72.3 mg, 0.258 mmol), Pd₂(dba)₃ (118 mg, 0.129 mmol) and PdCl₂(dppf)-CH₂Cl₂Adduct (105 mg, 0.129 mmol) to afford after workup and purification using method-AE, ethyl 2-(2-((5-(1-aminoisoquinolin-7-yl)-1-(tetrahydrofuran-3-yl)-1H-indazol-3-yl)methoxy)-6-(trifluoromethoxy)phenyl)acetate (288c) (283 mg, 36% yield) as a yellow solid; MS (ES+): 607.20 (M+1).

### Step-6: Preparation of 2-(2-((5-(1-aminoisoquinolin-7-yl)-1-(tetrahydrofuran-3-yl)-1H-indazol-3-yl)methoxy)-6-(trifluoromethoxy)phenyl)acetic acid (288d)

Compound 288d was prepared according to the procedure reported in step-2 of scheme-1, from ethyl 2-(2-(5-(1-aminoisoquinolin-7-yl)-1-(tetrahydrofuran-3-yl)-1H-indazol-3-yl)methoxy)-6-(trifluoromethoxy)phenyl)acetate (288c) (348 mg, 0.574 mmol) in THF (3 mL) using lithium hydroxide hydrate (72.2 mg, 1.721 mmol) in water (1 mL) to afford after work up and purification using method-M, 2-(2-((5-(1-aminoisoquinolin-7-yl)-1-(tetrahydrofuran-3-yl)-1H-indazol-3-yl)methoxy)-6-(trifluoromethoxy)phenyl)acetic acid (288d) (26.1 mg, 8% yield) HCl salt as a white solid; ¹H NMR (300 MHz, DMSO-d₆) δ 13.02 (s, 1H, D₂O exchangeable), 12.22 (s, 1H, DO exchangeable), 9.01 (s, 2H, D₂O exchangeable), 8.59 (d, J=8.3 Hz, 1H), 7.99-7.92 (m, 2H), 7.92-7.82 (m, 2H), 7.63 (d, J=7.2 Hz, 1H), 7.51 (dd, J=8.6, 1.6 Hz, 1H), 7.45-7.33 (m, 2H), 7.02-6.95 (m, 2H), 5.67-5.56 (m, 1H), 5.53 (s, 2H), 4.21-4.08 (m, 2H), 4.03-3.95 (m, 1H), 3.95-3.87 (m, 1H), 3.53 (s, 2H), 2.50-2.24 (m, 2H); ¹⁹F NMR (282 MHz, DMSO-d₆) δ −56.04; MS (ES+): 579.20 (M+1); (ES−): 577.10 (M−1).

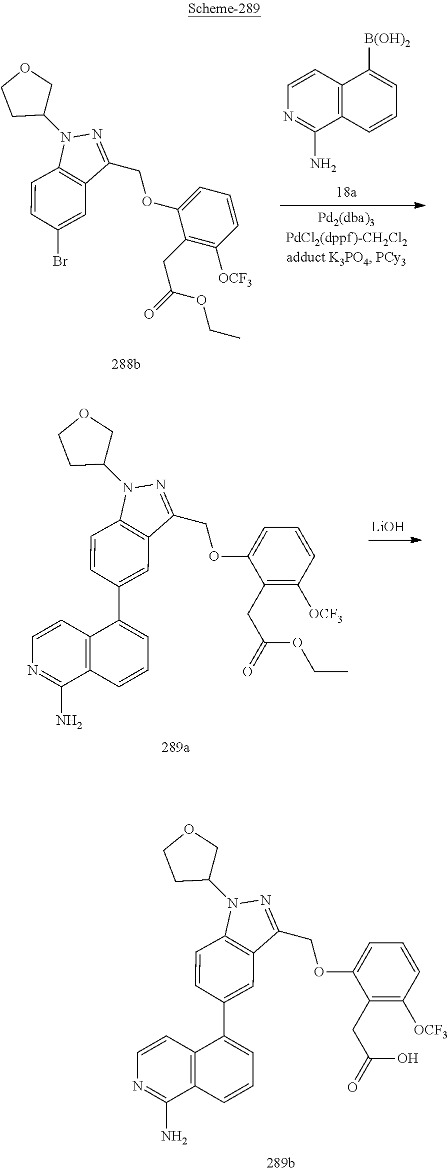 [SMILES: CCOC(=O)Cc1c(OCc2nn(C3CCOC3)c3ccc(Br)cc23)cccc1OC(F)(F)F ; Nc1nccc2c(B(O)O)cccc12 ; CCOC(=O)Cc1c(OCc2nn(C3CCOC3)c3ccc(-c4cccc5c(N)nccc45)cc23)cccc1OC(F)(F)F ; "LiOH" ; Nc1nccc2c(-c3ccc4c(c3)c(COc3cccc(OC(F)(F)F)c3CC(=O)O)nn4C3CCOC3)cccc12]

### Preparation of 2-(2-((5-(1-aminoisoquinolin-5-yl)-1-(tetrahydrofuran-3-yl)-1H-indazol-3-yl)methoxy)-6-(trifluoromethoxy)phenyl)acetic acid (289b)

### Step-1: Preparation of ethyl 2-(2-((5-(1-aminoisoquinolin-5-yl)-1-(tetrahydrofuran-3-yl)-1H-indazol-3-yl)methoxy)-6-(trifluoromethoxy)phenyl)acetate (289a)

Compound 289a was prepared according to the procedure reported in step-2 of scheme-18, from ethyl 2-(2-((5-bromo-1-(tetrahydrofuran-3-yl)-1H-indazol-3-yl)methoxy)-6-(trifluoromethoxy)phenyl)acetate (288b) (700 mg, 1.288 mmol) in dioxane/MeTHF (10 mL, Ratio: 1:1) using (1-aminoisoquinolin-5-yl)boronic acid (18a) (315 mg, 1.675 mmol), aqueous tripotassium phosphate (2M, 1288 μl, 5.15 mmol), tricyclohexylphosphine (72.3 mg, 0.258 mmol) Pd₂(dba)₃ (118 mg, 0.129 mmol) and PdCl₂(dppf)-CH₂Cl₂Adduct (105 mg, 0.129 mmol) to afford after workup and purification using method-AE, ethyl 2-(2-((5-(1-aminoisoquinolin-5-yl)-1-(tetrahydrofuran-3-yl)-1H-indazol-3-yl)methoxy)-6-(trifluoromethoxy)phenyl)acetate (289a) (156 mg, 20% yield) as a yellow solid; MS (ES+): 607.20 (M+1).

### Step-3: Preparation of 2-(2-((5-(1-aminoisoquinolin-5-yl)-1-(tetrahydrofuran-3-yl)-1H-indazol-3-yl)methoxy)-6-(trifluoromethoxy)phenyl)acetic acid (289b)

Compound 289b was prepared according to the procedure reported in step-2 of scheme-1, from ethyl 2-(2-((5-(1-aminoisoquinolin-5-yl)-1-(tetrahydrofuran-3-yl)-1H-indazol-3-yl)methoxy)-6-(trifluoromethoxy)phenyl)acetate (289a) (245 mg, 0.404 mmol) in THF (3 mL) using lithium hydroxide hydrate (50.8 mg, 1.212 mmol) in water (1 mL) to afford after work up and purification using method-M, 2-(2-((5-(1-aminoisoquinolin-5-yl)-1-(tetrahydrofuran-3-yl)-1H-indazol-3-yl)methoxy)-6-(trifluoromethoxy)phenyl)acetic acid (289b) (25.0 mg, 11% yield) HCl salt as a white solid; ¹H NMR (300 MHz, DMSO-d₆) δ 13.20 (s, 1H, D₂O exchangeable), 12.23 (s, 1H, D₂O exchangeable), 9.10 (s, 2H, D₂O exchangeable), 8.61 (d, J=8.3 Hz, 1H), 8.00-7.79 (m, 4H), 7.63 (d, J=7.2 Hz, 1H), 7.50 (d, J=8.7 Hz, 1H), 7.44-7.32 (m, 2H), 6.97 (d, J=7.3 Hz, 2H), 5.65-5.52 (m, 1H), 5.52 (s, 2H), 4.21-4.07 (m, 2H), 4.04-3.94 (m, 1H), 3.94-3.87 (m, 1H), 3.52 (s, 2H), 2.43-2.30 (m, 2H); 1ºF NMR (282 MHz, DMSO-d₆) δ −56.04; MS (ES+): 579.20 (M+1); (ES−): 577.10 (M−1).

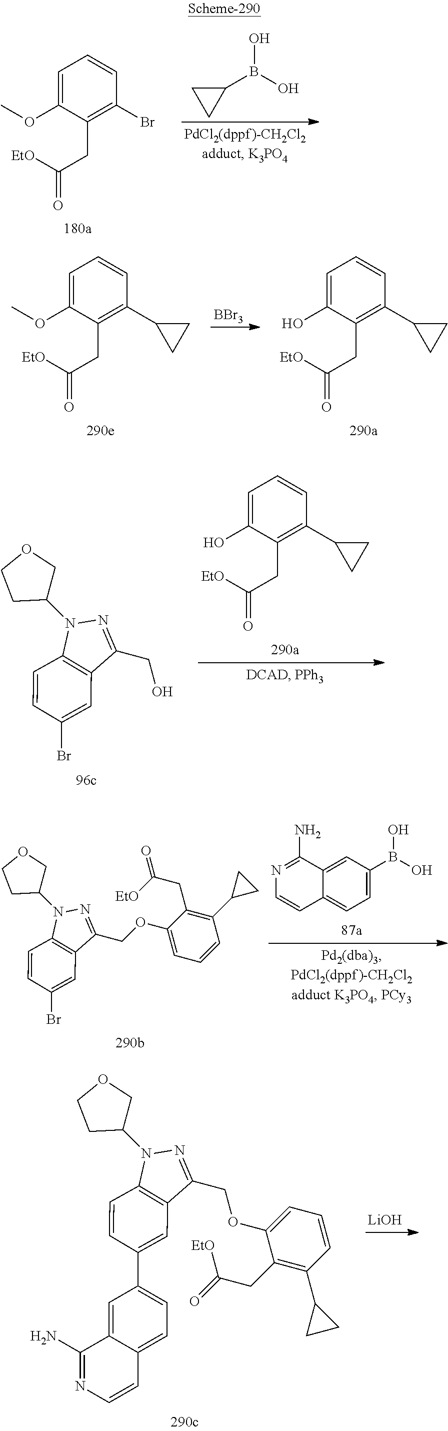

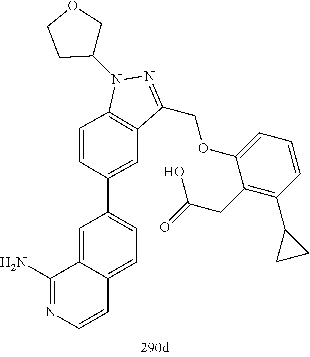 [SMILES: Nc1nccc2ccc(-c3ccc4c(c3)c(COc3cccc(C5CC5)c3CC(=O)O)nn4C3CCOC3)cc12]

### Preparation of 2-(2-((5-(1-aminoisoquinolin-7-yl)-1-(tetrahydrofuran-3-yl)-1H-indazol-3-yl)methoxy)-6-cyclopropylphenyl)acetic acid (290d)

### Step-1: Preparation of ethyl 2-(2-cyclopropyl-6-methoxyphenyl)acetate (290e)

Compound 290e was prepared according to the procedure reported in step-1 of scheme-180, from ethyl 2-(2-bromo-6-methoxyphenyl)acetate (180a)(30 g, 109.84 mmol) in 1,4-dioxane (300 mL) using cyclopropyl boronic acid (14.15 g, 164.7 mmol), K₃PO₄ (58.28g, 274.6 mmol), PdCl₂(dppf)-CH₂Cl₂ adduct (1.79 g, 2.19 mmol) and heating at 90° C. for 16 h to afford after work up and purification using column chromatography [silica gel, eluting with EtOAc in n-heptane] ethyl 2-(2-cyclopropyl-6-methoxyphenyl)acetate (290e)(12 g, 47% yield) as a light yellow liquid; ¹H NMR (300 MHz, Chloroform-d) δ 7.21 (td, J=8.0, 1.2 Hz, 1H), 6.77 (dd, J=12.7, 7.9 Hz, 2H), 4.20 (qd, J=7.1, 1.3 Hz, 2H), 3.95 (d, J=1.2 Hz, 2H), 3.84 (d, J=1.3 Hz, 3H), 1.98-1.85 (m, 1H), 1.29 (td, J=7.1, 1.3 Hz, 3H), 1.00-0.89 (m, 2H), 0.73-0.61 (m, 2H).

### Step-2: Preparation of ethyl 2-(2-cyclopropyl-6-hydroxyphenyl)acetate (290a)

Compound 290a was prepared according to the procedure reported in step-1 of scheme-94, from ethyl 2-(2-cyclopropyl-6-methoxyphenyl)acetate (290e) (12 g, 51.22 mmol) in DCM (187.2 mL) using BBr₃ in DCM (12.83 g, 51.22 mmol) stirring at −78° C. for 1 h and at RT for 2 h to afford after work up and purification using column chromatography [silica gel, eluting with EtOAc in n-heptane] ethyl 2-(2-cyclopropyl-6-hydroxyphenyl)acetate (290a) (5.1 g, 45% yield) as a light yellow liquid; ¹H NMR (300 MHz, DMSO-d₆) δ 9.39 (s, 1H), 6.96 (t, J=7.9 Hz, 1H), 6.65 (dd, J=8.1, 1.2 Hz, 1H), 6.47 (dt, J=7.6, 1.0 Hz, 1H), 4.06 (q, J=7.1 Hz, 2H), 3.76 (s, 2H), 1.87-1.74 (m, 1H), 1.17 (t, J=7.1 Hz, 3H), 0.89-0.77 (m, 2H), 0.57-0.47 (m, 2H).

### Step-3: Preparation of ethyl 2-(2-((5-bromo-1-(tetrahydrofuran-3-yl)-1H-indazol-3-yl)methoxy)-6-cyclopropylphenyl)acetate (290b)

Compound 290b was prepared according to the procedure reported in step-2 of scheme-2, from (5-bromo-1-(tetrahydrofuran-3-yl)-1H-indazol-3-yl)methanol (96c) (3.0 g, 10.10 mmol) in DCM (50 mL) using ethyl 2-(2-cyclopropyl-6-hydroxyphenyl)acetate (290a) (2.67 g, 12.12 mmol), PPh₃ (2.91 g, 11.11 mmol) a solution of DCAD (4.08 g, 11.11 mmol) in DCM (20 mL) to afford after workup and purification using method-AU, ethyl 2-(2-((5-bromo-1-(tetrahydrofuran-3-yl)-1H-indazol-3-yl)methoxy)-6-cyclopropylphenyl)acetate (290b) (1.652 g, 33% yield) as a white solid; MS (ES+): 499.10 (M+1).

### Step-4: Preparation of ethyl 2-(2-((5-(1-aminoisoquinolin-7-yl)-1-(tetrahydrofuran-3-yl)-1H-indazol-3-yl)methoxy)-6-cyclopropylphenyl)acetate (290c)

Compound 290c was prepared according to the procedure reported in step-2 of scheme-18, from ethyl 2-(2-((5-bromo-1-(tetrahydrofuran-3-yl)-1H-indazol-3-yl)methoxy)-6-cyclopropylphenyl)acetate (290b) (700 mg, 1.402 mmol) in dioxane/MeTHF (10 mL, Ratio: 1:1) using (1-aminoisoquinolin-7-yl)boronic acid (87a) (343 mg, 1.822 mmol), aqueous tripotassium phosphate (2M, 1402 μl, 5.61 mmol), tricyclohexylphosphine (79 mg, 0.280 mmol), Pd₂(dba)₃ (128 mg, 0.140 mmol) and PdCl₂(dppf)-CH₂Cl₂Adduct (114 mg, 0.14 mmol) to afford after workup and purification using method-AE, ethyl 2-(2-((5-(1-aminoisoquinolin-7-yl)-1-(tetrahydrofuran-3-yl)-1H-indazol-3-yl)methoxy)-6-cyclopropylphenyl)acetate (290c) (283 mg, 36% yield) as a yellow solid; MS (ES+): 563.30 (M+1).

### Step-5: Preparation of 2-(2-((5-(1-aminoisoquinolin-7-yl)-1-(tetrahydrofuran-3-yl)-1H-indazol-3-yl)methoxy)-6-cyclopropylphenyl)acetic acid (290d)

Compound 290d was prepared according to the procedure reported in step-2 of scheme-1, from ethyl 2-(2-((5-(1-aminoisoquinolin-7-yl)-1-(tetrahydrofuran-3-yl)-1H-indazol-3-yl)methoxy)-6-cyclopropylphenyl)acetate (290c) (368 mg, 0.654 mmol) in THF (3 mL) using lithium hydroxide hydrate (27.4 mg, 0.654 mmol) in water (1 mL) to afford after work up and purification using method-M, 2-(2-((5-(1-aminoisoquinolin-7-yl)-1-(tetrahydrofuran-3-yl)-1H-indazol-3-yl)methoxy)-6-cyclopropylphenyl)acetic acid (290d) (64.2 mg, 18% yield) HCl salt as a white solid; ¹H NMR (300 MHz, DMSO-d₆) δ 13.25 (s, 1H, D₂O exchangeable), 12.12 (s, 1H, D₂O exchangeable), 9.15 (s, 2H, D₂O exchangeable), 8.96 (s, 1H), 8.40 (d, J=8.5 Hz, 1H), 8.30 (s, 1H), 8.04 (t, 2H), 8.00-7.92 (m, 1H), 7.70 (d, J=6.9 Hz, 1H), 7.28 (d, J=6.9 Hz, 1H), 7.22-7.13 (m, 2H), 6.67 (d, J=6.7 Hz, 1H), 5.66-5.54 (m, 1H), 5.47 (s, 2H), 4.21-4.06 (m, 2H), 4.02-3.88 (m, 2H), 3.76 (s, 2H), 2.49-2.25 (m, 2H), 1.90-1.78 (m, 1H), 0.90-0.81 (m, 2H), 0.63-0.51 (m, 2H); MS (ES+): 535.20 (M+1); (ES−): 533.10 (M−1).

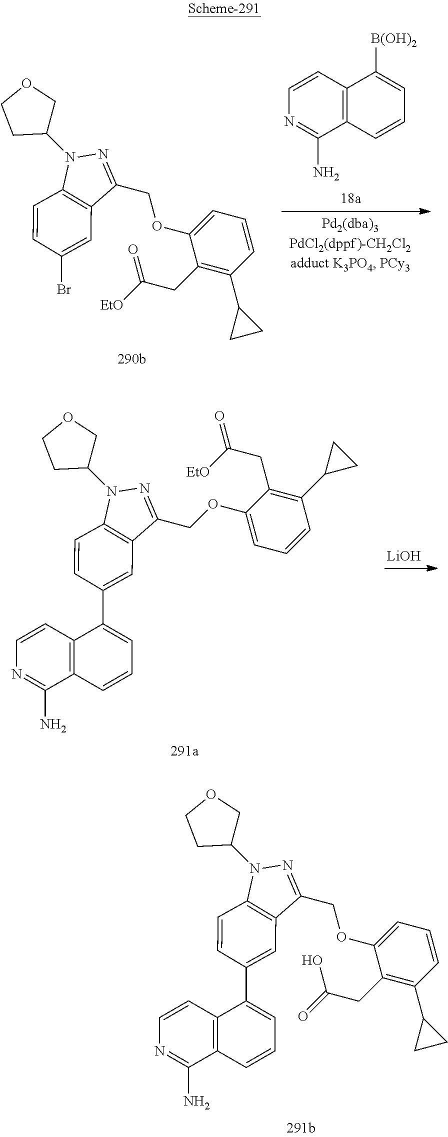 [SMILES: CCOC(=O)Cc1c(OCc2nn(C3CCOC3)c3ccc(Br)cc23)cccc1C1CC1 ; Nc1nccc2c(B(O)O)cccc12 ; CCOC(=O)Cc1c(OCc2nn(C3CCOC3)c3ccc(-c4cccc5c(N)nccc45)cc23)cccc1C1CC1 ; "LiOH" ; Nc1nccc2c(-c3ccc4c(c3)c(COc3cccc(C5CC5)c3CC(=O)O)nn4C3CCOC3)cccc12]

### Preparation of 2-(2-((5-(1-aminoisoquinolin-5-yl)-1-(tetrahydrofuran-3-yl)-1H-indazol-3-yl)methoxy)-6-cyclopropylphenyl)acetic acid (291b)

### Step-1: Preparation of ethyl 2-(2-((5-(1-aminoisoquinolin-5-yl)-1-(tetrahydrofuran-3-yl)-1H-indazol-3-yl)methoxy)-6-cyclopropylphenyl)acetate (291a)

Compound 291a was prepared according to the procedure reported in step-2 of scheme-18, from ethyl 2-(2-((5-bromo-1-(tetrahydrofuran-3-yl)-1H-indazol-3-yl)methoxy)-6-cyclopropylphenyl)acetate (290b) (700 mg, 1.402 mmol) in dioxane/MeTHF (10 mL, Ratio: 1:1) using (1-aminoisoquinolin-5-yl)boronic acid (18a) (343 mg, 1.822 mmol), aqueous tripotassium phosphate (2M) (1402 μl, 5.61 mmol), tricyclohexylphosphine (79 mg, 0.280 mmol), Pd₂(dba)₃ (128 mg, 0.140 mmol) and PdCl₂(dppf)-CH₂Cl₂Adduct (114 mg, 0.140 mmol) to afford after workup and purification using method-AE, ethyl 2-(2-((5-(1-aminoisoquinolin-5-yl)-1-(tetrahydrofuran-3-yl)-1H-indazol-3-yl)methoxy)-6-cyclopropylphenyl)acetate (291a) (156 mg, 20% yield) as a yellow solid; MS (ES+): 563.30 (M+1).

### Step-2: Preparation of 2-(2-((5-(1-aminoisoquinolin-5-yl)-1-(tetrahydrofuran-3-yl)-1H-indazol-3-yl)methoxy)-6-cyclopropylphenyl)acetic acid (291b)

Compound 291b was prepared according to the procedure reported in step-2 of scheme-1, from ethyl 2-(2-((5-(1-aminoisoquinolin-5-yl)-1-(tetrahydrofuran-3-yl)-1H-indazol-3-yl)methoxy)-6-cyclopropylphenyl)acetate (291a) (272 mg, 0.483 mmol) in THF (3 mL) using lithium hydroxide hydrate (20.28 mg, 0.483 mmol) in water (1 mL) to afford after work up and purification using method-M, 2-(2-((5-(1-aminoisoquinolin-5-yl)-1-(tetrahydrofuran-3-yl)-1H-indazol-3-yl)methoxy)-6-cyclopropylphenyl)acetic acid (291b) (43.6 mg, 17% yield) HCl salt as a white solid; ¹H NMR (300 MHz, DMSO-d₆) δ 13.28 (s, 1H, D₂O exchangeable), 12.02 (s, 1H, D₂O exchangeable), 9.17 (s, 2H, D₂O exchangeable), 8.63 (d, J=8.3 Hz, 1H), 7.99-7.91 (m, 2H), 7.91-7.83 (m, 2H), 7.64 (d, J=7.2 Hz, 1H), 7.51 (d, J=8.7 Hz, 1H), 7.20-7.08 (m, 2H), 6.98 (d, J=7.2 Hz, 1H), 6.64 (d, J=6.8 Hz, 1H), 5.67-5.55 (m, 1H), 5.43 (s, 2H), 4.21-4.09 (m, 2H), 4.04-3.96 (m, 1H), 3.96-3.87 (m, 1H), 3.71 (s, 2H), 2.49-2.31 (m, 2H), 1.87-1.76 (m, 1H), 0.89-0.80 (m, 2H), 0.58-0.49 (m, 2H); MS (ES+): 535.20 (M+1); (ES−): 533.20 (M−1).

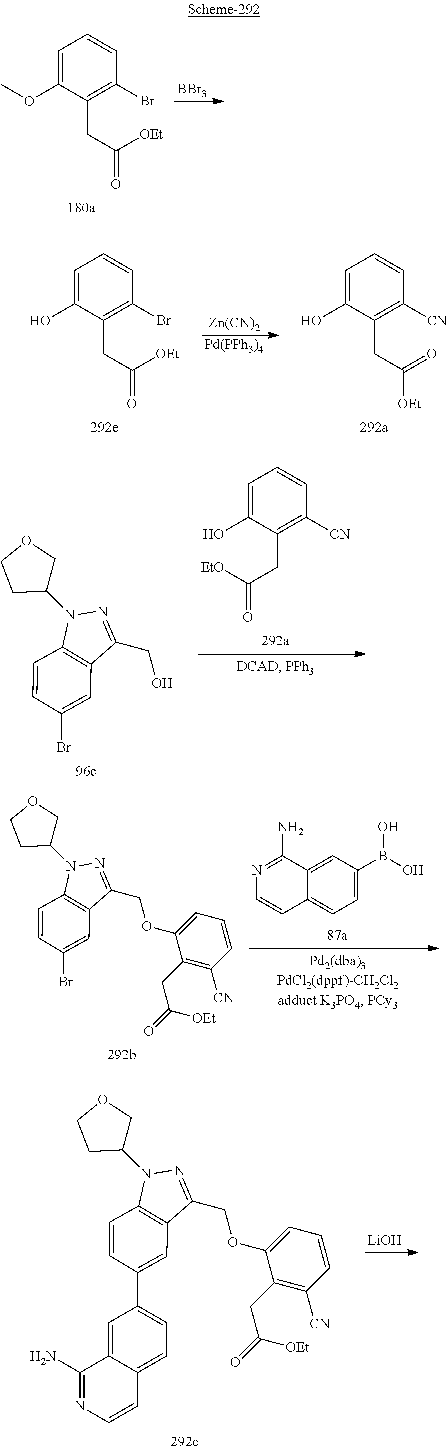

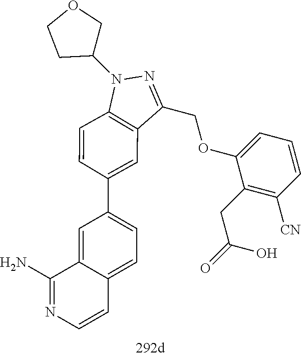 [SMILES: N#Cc1cccc(OCc2nn(C3CCOC3)c3ccc(-c4ccc5ccnc(N)c5c4)cc23)c1CC(=O)O]

### Preparation of 2-(2-((5-(1-aminoisoquinolin-7-yl)-1-(tetrahydrofuran-3-yl)-1H-indazol-3-yl)methoxy)-6-cyanophenyl)acetic acid (292d)

### Step-1: Preparation of ethyl 2-(2-bromo-6-hydroxyphenyl)acetate (292e)

Compound 292e was prepared according to the procedure reported in step-1 of scheme-94, from ethyl 2-(2-bromo-6-methoxyphenyl)acetate (180a) (27 g, 98.85 mmol) in DCM (421.2 mL) using BBr₃ in DCM (49.52 g, 197.7 mmol) stirring at −78 º C for 1 h and at RT for 3 h to afford after work up and purification using column chromatography [silica gel, eluting with EtOAc in heptane] ethyl 2-(2-bromo-6-hydroxyphenyl)acetate (292e) (19.5 g, 76% yield) as pale yellow solid; ¹H NMR (300 MHz, Chloroform-d) δ 7.32-7.14 (m, 1H), 7.07 (dt, J=8.0, 1.2 Hz, 1H), 6.90 (td, J=8.1, 1.1 Hz, 1H), 6.70 (dt, J=8.1, 1.2 Hz, 1H), 4.15 (qd, J=7.2, 1.1 Hz, 2H), 3.86 (d, J=1.1 Hz, 2H), 1.23 (td, J=7.1, 1.1 Hz, 3H).

### Step-2: Preparation of ethyl 2-(2-cyano-6-hydroxyphenyl)acetate (292a)

To a stirred solution of ethyl 2-(2-bromo-6-hydroxyphenyl)acetate (292e) (19.5 g, 75.26 mmol) in DMF (195 mL) was added Zn(CN)₂ (8.84 g, 75.26 mmol), Pd(PPh₃)+(8.69 g, 7.52 mmol) at RT and the mixture was purged with N₂ for 15 minute and heated at 120° C. overnight. Reaction mixture was cooled to RT, filtered through a pad of Celite and the filtrate was extracted with EtOAc (3×100 mL). Combined organic layer were washed with brine, dried, filtered and concentrated in vacuo. The residue obtained was purified using column chromatography [silica gel, eluting with EtOAc in n-heptane] to afford ethyl 2-(2-cyano-6-hydroxyphenyl)acetate (292a) (5 g, 32% yield) as a light yellow solid; ¹H NMR (300 MHz, Chloroform-d) δ 7.90 (s, 1H), 7.31-7.22 (m, 2H), 7.18-7.09 (m, 1H), 4.27 (qd, J=7.2, 1.0 Hz, 2H), 3.97 (d, J=1.1 Hz, 2H), 1.34 (td, J=7.1, 1.1 Hz, 3H).

### Step-3: Preparation of ethyl 2-(2-((5-bromo-1-(tetrahydrofuran-3-yl)-1H-indazol-3-yl)methoxy)-6-cyanophenyl)acetate (292b)

Compound 292b was prepared according to the procedure reported in step-2 of scheme-2, from (5-bromo-1-(tetrahydrofuran-3-yl)-1H-indazol-3-yl)methanol (96c) (7.5 g, 25.2 mmol) in DCM (50 mL) using ethyl 2-(2-cyano-6-hydroxyphenyl)acetate (292a) (6.22 g, 30.3 mmol), PPh₃ (6.22 g, 30.3 mmol) a solution of DCAD (10.19 g, 27.8 mmol) in DCM (15 mL) to afford after workup and purification using method-AU, ethyl 2-(2-((5-bromo-1-(tetrahydrofuran-3-yl)-1H-indazol-3-yl)methoxy)-6-cyanophenyl)acetate (292b) (3.65 g, 30% yield) as a white solid; MS (ES+): 485.10 (M+1).

### Step-4: Preparation of ethyl 2-(2-((5-(1-aminoisoquinolin-7-yl)-1-(tetrahydrofuran-3-yl)-1H-indazol-3-yl)methoxy)-6-cyanophenyl)acetate (292c)

Compound 292c was prepared according to the procedure reported in step-2 of scheme-18, from ethyl 2-(2-((5-bromo-1-(tetrahydrofuran-3-yl)-1H-indazol-3-yl)methoxy)-6-cyanophenyl)acetate (292b) (1.4 g, 2.89 mmol) in dioxane/MeTHF (10 mL, Ratio: 1:1) using (1-aminoisoquinolin-7-yl)boronic acid (87a) (0.706 g, 3.76 mmol), aqueous tripotassium phosphate (2M, 2.89 mL, 11.56 mmol), tricyclohexylphosphine (0.162 g, 0.578 mmol), Pd₂(dba)₃ (0.265 g, 0.289 mmol) and PdCl₂(dppf)-CH₂Cl₂Adduct (0.236 g, 0.289 mmol) to afford after workup and purification using method-AE, ethyl 2-(2-((5-(1-aminoisoquinolin-7-yl)-1-(tetrahydrofuran-3-yl)-1H-indazol-3-yl)methoxy)-6-cyanophenyl)acetate (292c) (963 mg, 61% yield) as a yellow solid; MS (ES+): 548.30 (M+1).

### Step-5: Preparation of 2-(2-((5-(1-aminoisoquinolin-7-yl)-1-(tetrahydrofuran-3-yl)-1H-indazol-3-yl)methoxy)-6-cyanophenyl)acetic acid (292d)

Compound 292d was prepared according to the procedure reported in step-2 of scheme-1, from ethyl 2-(2-((5-(1-aminoisoquinolin-7-yl)-1-(tetrahydrofuran-3-yl)-1H-indazol-3-yl)methoxy)-6-cyanophenyl)acetate (292c) (300 mg, 0.548 mmol) in THF (3 mL) using lithium hydroxide (13.12 mg, 0.548 mmol) in water (1 mL) to afford after work up and purification using method-M, 2-(2-((5-(1-aminoisoquinolin-7-yl)-1-(tetrahydrofuran-3-yl)-1H-indazol-3-yl)methoxy)-6-cyanophenyl)acetic acid (292d) (39.9 mg, 14% yield) HCl salt as a white solid; ¹H NMR (300 MHz, DMSO-d₆) δ 13.06 (s, 1H, DO exchangeable), 12.57 (s, 1H, D₂O exchangeable), 9.10 (s, 2H, D₂O exchangeable), 8.95 (s, 1H), 8.42 (d, J=8.5 Hz, 1H), 8.34 (s, 1H), 8.05 (t, 2H), 8.00-7.91 (m, 1H), 7.74-7.64 (m, 2H), 7.56-7.39 (m, 2H), 7.28 (d, J=6.9 Hz, 1H), 5.61-5.55 (m, 3H), 4.21-4.04 (m, 2H), 4.01-3.85 (m, 2H), 3.73 (s, 2H), 2.43-2.15 (m, 2H); MS (ES+): 520.20 (M+1); FT-1R; 2227 cm⁻¹.

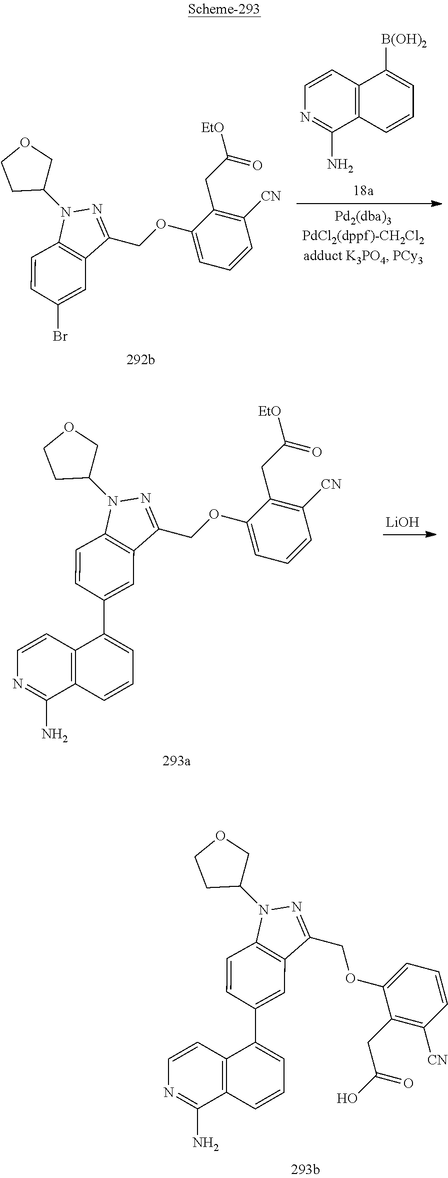 [SMILES: CCOC(=O)Cc1c(C#N)cccc1OCc1nn(C2CCOC2)c2ccc(Br)cc12 ; Nc1nccc2c(B(O)O)cccc12 ; CCOC(=O)Cc1c(C#N)cccc1OCc1nn(C2CCOC2)c2ccc(-c3cccc4c(N)nccc34)cc12 ; "LiOH" ; N#Cc1cccc(OCc2nn(C3CCOC3)c3ccc(-c4cccc5c(N)nccc45)cc23)c1CC(=O)O]

### Preparation of 2-(2-((5-(1-aminoisoquinolin-5-yl)-1-(tetrahydrofuran-3-yl)-1H-indazol-3-yl)methoxy)-6-cyanophenyl)acetic acid (293b)

### Step-1: Preparation of ethyl 2-(2-((5-(1-aminoisoquinolin-5-yl)-1-(tetrahydrofuran-3-yl)-1H-indazol-3-yl)methoxy)-6-cyanophenyl)acetate (293a)

Compound 293a was prepared according to the procedure reported in step-2 of scheme-18, from ethyl 2-(2-((5-bromo-1-(tetrahydrofuran-3-yl)-1H-indazol-3-yl)methoxy)-6-cyanophenyl)acetate (292b) (1.4 g, 2.89 mmol) in dioxane/MeTHF (10 mL, Ratio: 1:1) using (I-aminoisoquinolin-5-yl)boronic acid (18a) (0.706 g, 3.76 mmol), aqueous tripotassium phosphate (2M, 2.89 mL, 11.56 mmol), tricyclohexylphosphine (0.162 g, 0.578 mmol) Pd₂(dba)₃ (0.265 g, 0.289 mmol) and PdCl₂(dppf)-CH₂Cl₂Adduct (0.236 g, 0.289 mmol) to afford after workup and purification using method-AE, ethyl 2-(2-((5-(1-aminoisoquinolin-5-yl)-1-(tetrahydrofuran-3-yl)-1H-indazol-3-yl)methoxy)-6-cyanophenyl)acetate (293a) (764 mg, 48% yield) as a yellow solid; MS (ES+): 548.30 (M+1).

### Step-2: Preparation of 2-(2-((5-(1-aminoisoquinolin-5-yl)-1-(tetrahydrofuran-3-yl)-1H-indazol-3-yl)methoxy)-6-cyanophenyl)acetic acid (293b)

Compound 293b was prepared according to the procedure reported in step-2 of scheme-1, ethyl 2-(2-((5-(1-aminoisoquinolin-5-yl)-1-(tetrahydrofuran-3-yl)-1H-indazol-3-yl)methoxy)-6-cyanophenyl)acetate (293a) (0.150 g, 0.274 mmol) in THF (3 mL) using lithium hydroxide (6.56 mg, 0.274 mmol) in water (1 mL) to afford after work up and purification using method-M, 2-(2-((5-(1-aminoisoquinolin-5-yl)-1-(tetrahydrofuran-3-yl)-1H-indazol-3-yl)methoxy)-6-cyanophenyl)acetic acid (293b) (31.2 mg, 22% yield) HCl salt as a white solid; ¹H NMR (300 MHz, DMSO-d₆) δ 13.22 (s, 1H, DO exchangeable), 12.61 (s, 1H, D₂O exchangeable), 9.09 (s, 2H, D₂O exchangeable), 8.61 (d, J=8.3 Hz, 1H), 8.03-7.91 (m, 2H), 7.91-7.78 (m, 2H), 7.72-7.60 (m, 2H), 7.53 (t, J=7.9 Hz, 1H), 7.51-7.42 (m, 1H), 7.33 (d, J=8.9 Hz, 1H), 6.97 (d, J=7.3 Hz, 1H), 5.77 (s, 2H), 5.58-5.52 (m, 1H), 4.25-4.12 (m, 2H), 4.03-3.86 (m, 2H), 3.69 (s, 2H), 2.48-2.40 (m, 2H); MS (ES+): 520.20 (M+1); FT−1R; 2229 cm⁻¹.

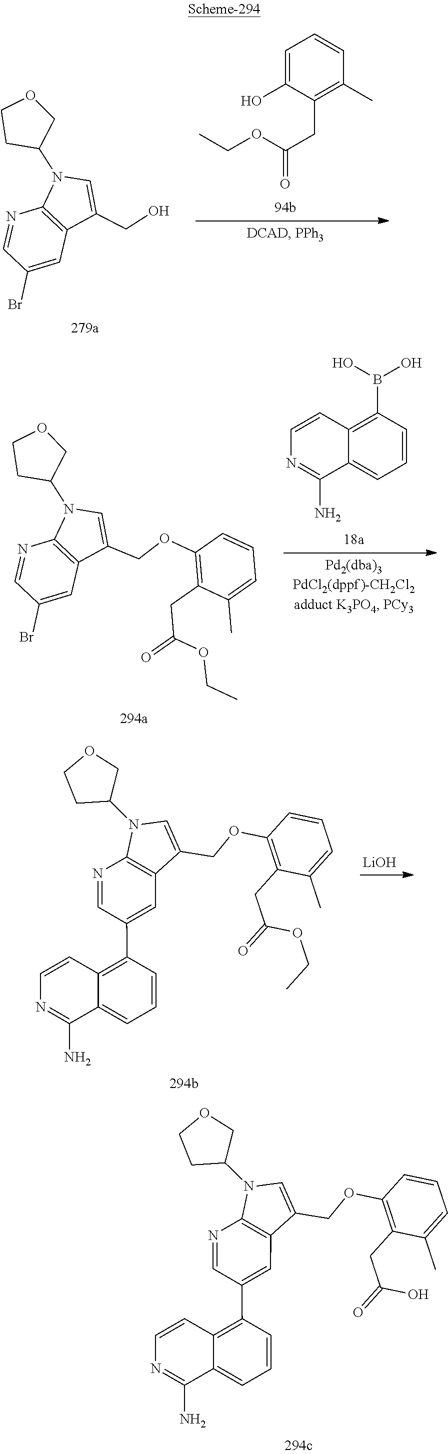

### Preparation of 2-(2-((5-(1-aminoisoquinolin-5-yl)-1-(tetrahydrofuran-3-yl)-1H-pyrrolo[2,3-b]pyridin-3-yl)methoxy)-6-methylphenyl)acetic acid (294c)

### Step-1: Preparation of ethyl 2-(2-((5-bromo-1-(tetrahydrofuran-3-yl)-1H-pyrrolo[2,3-b]pyridin-3-yl)methoxy)-6-methylphenyl)acetate (294a)

Compound 294a was prepared according to the procedure reported in step-2 of scheme-2, from (5-bromo-1-(tetrahydrofuran-3-yl)-1H-pyrrolo[2,3-b]pyridin-3-yl)methanol (279a) (800 mg, 2.69 mmol) in DCM (10 mL) using ethyl 2-(2-hydroxy-6-methylphenyl)acetate (94b) (800 mg, 2.69 mmol), PPh₃ (1059 mg, 4.04 mmol) a solution of DCAD (1483 mg, 4.04 mmol) in DCM (10 mL) to afford after workup and purification using method-S, ethyl 2-(2-((5-bromo-1-(tetrahydrofuran-3-yl)-1H-pyrrolo[2,3-b]pyridin-3-yl)methoxy)-6-methylphenyl)acetate (294a) (800 mg, 63% yield) as a white oil; MS (ES+): 473.10 & 475.10 (M+1).

### Step-2: Preparation of ethyl 2-(2-((5-(1-aminoisoquinolin-5-yl)-1-(tetrahydrofuran-3-yl)-1H-pyrrolo[2,3-b]pyridin-3-yl)methoxy)-6-methylphenyl)acetate (294b)

Compound 294b was prepared according to the procedure reported in step-2 of scheme-18, from ethyl 2-(2-((5-bromo-1-(tetrahydrofuran-3-yl)-1H-pyrrolo[2,3-b]pyridin-3-yl)methoxy)-6-methylphenyl)acetate (294a) (700 mg, 1.479 mmol) in dioxane/THF (8 mL, Ratio: 1:1) using (1-aminoisoquinolin-5-yl)boronic acid (18a) (417 mg, 2.218 mmol), aqueous tripotassium phosphate (2M, 2.96 mL, 5.92 mmol), tricyclohexylphosphine (83 mg, 0.296 mmol), Pd₂(dba)₃ (135 mg, 0.148 mmol) and PdCl₂(dppf)-CH₂Cl₂Adduct (121 mg, 0.148 mmol) heating at 90° C. for 2 h to afford after workup and purification using method-AM, ethyl 2-(2-((5-(1-aminoisoquinolin-5-yl)-1-(tetrahydrofuran-3-yl)-1H-pyrrolo[2,3-b]pyridin-3-yl)methoxy)-6-methylphenyl)acetate (294b) (300 mg, 38% yield) as a clear oil; MS (ES+): 537.30 (M+1).

### Step-3: Preparation of 2-(2-((5-(1-aminoisoquinolin-5-yl)-1-(tetrahydrofuran-3-yl)-1H-pyrrolo[2,3-b]pyridin-3-yl)methoxy)-6-methylphenyl)acetic acid (294c)

Compound 294c was prepared according to the procedure reported in step-2 of scheme-1, ethyl 2-(2-((5-(1-aminoisoquinolin-5-yl)-1-(tetrahydrofuran-3-yl)-1H-pyrrolo[2,3-b]pyridin-3-yl)methoxy)-6-methylphenyl)acetate (294b) (300 mg, 0.559 mmol) in MeOH/THF (6 mL) using lithium hydroxide hydrate (70.4 mg, 1.677 mmol) in water (2 mL) to afford after work up and purification using method-G, 2-(2-((5-(1-aminoisoquinolin-5-yl)-1-(tetrahydrofuran-3-yl)-1H-pyrrolo[2,3-b]pyridin-3-yl)methoxy)-6-methylphenyl)acetic acid (294c) (95 mg, 33% yield) HCl salt as a white solid; ¹H NMR (300 MHz, DMSO-d₆) δ 8.45 (d, J=8.3 Hz, 1H), 8.40-8.31 (m, 1H), 8.20-7.99 (m, 3H, 2H exchangeable), 7.88-7.78 (m, 2H), 7.78-7.66 (m, 2H), 7.13 (t, J=7.8 Hz, 1H), 7.08-7.01 (m, 1H), 6.90 (d, J=6.7 Hz, 1H), 6.78 (d, J=7.4 Hz, 1H), 5.66-5.52 (m, 1H), 5.26 (s, 2H), 4.22-3.99 (m, 2H), 3.99-3.82 (m, 2H), 3.55 (s, 2H), 2.62-2.52 (m, 1H), 2.29-2.20 (m, 1H), 2.18 (s, 3H); MS (ES+): 509.20 (M+1); (ES−): 507.20 (M−1).

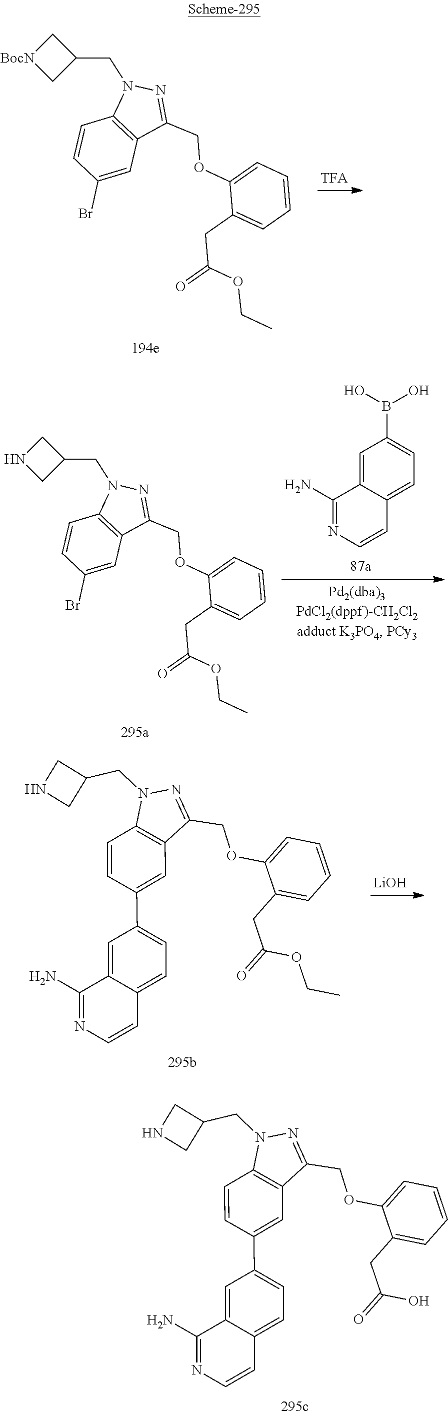

### Preparation of 2-(2-((5-(1-aminoisoquinolin-7-yl)-1-(azetidin-3-ylmethyl)-1H-indazol-3-yl)methoxy)phenyl)acetic acid (295c)

### Step-1: Preparation of ethyl 2-(2-((1-(azetidin-3-ylmethyl)-5-bromo-1H-indazol-3-yl)methoxy)phenyl)acetate (295a)

Compound 295a was prepared according to the procedure reported in step-4 of scheme-9, from tert-butyl 3-((5-bromo-3-((2-(2-ethoxy-2-oxoethyl)phenoxy)methyl)-1H-indazol-1-yl)methyl)azetidine-1-carboxylate (194e) (6.05 g, 10.83 mmol) in DCM (100 mL) using TFA (8.35 mL, 108 mmol) and stirring at RT for 16 h to afford after work up ethyl 2-(2-((1-(azetidin-3-ylmethyl)-5-bromo-1H-indazol-3-yl)methoxy)phenyl)acetate (295a) (4.541 g, 91% yield) and was used as such for the next step; (ES+): 458.1 (M+1).

### Step-2: Preparation of ethyl 2-(2-((5-(1-aminoisoquinolin-7-yl)-1-(azetidin-3-ylmethyl)-1H-indazol-3-yl)methoxy)phenyl)acetate (295b)

Compound 295b was prepared according to the procedure reported in step-2 of scheme-18, from ethyl 2-(2-((1-(azetidin-3-ylmethyl)-5-bromo-1H-indazol-3-yl)methoxy)phenyl)acetate (295a) (300 mg, 0.655 mmol) in dioxane/MeTHF (10 mL, Ratio: 1:1) using (1-aminoisoquinolin-7-yl)boronic acid (87a) (160 mg, 0.851 mmol), aqueous tripotassium phosphate (4 M, 655 μl mL, 2.62 mmol), tricyclohexylphosphine (36.7 mg, 0.131 mmol), Pd₂(dba)₃ (59.9 mg, 0.065 mmol) and PdCl₂(dppf)-CH₂Cl₂Adduct (53.4 mg, 0.065 mmol) to afford after workup and purification using method-AE, ethyl 2-(2-((5-(1-aminoisoquinolin-7-yl)-1-(azetidin-3-ylmethyl)-1H-indazol-3-yl)methoxy)phenyl)acetate (295b) (217 mg, 64% yield) as a yellow solid; MS (ES+): 522.2 (M+1).

### Step-3: Preparation of 2-(2-((5-(1-aminoisoquinolin-7-yl)-1-(azetidin-3-ylmethyl)-1H-indazol-3-yl)methoxy)phenyl)acetic acid (295c)

Compound 295c was prepared according to the procedure reported in step-2 of scheme-1, from ethyl 2-(2-((5-(1-aminoisoquinolin-7-yl)-1-(azetidin-3-ylmethyl)-1H-indazol-3-yl)methoxy)phenyl)acetate (295b) (80 mg, 0.153 mmol) in THF (3 mL) using lithium hydroxide hydrate (19.31 mg, 0.460 mmol) in water (1 mL) to afford after work up and purification using method-M, 2-(2-((5-(1-aminoisoquinolin-7-yl)-1-(azetidin-3-ylmethyl)-1H-indazol-3-yl)methoxy)phenyl)acetic acid (295c) (5.3 mg, 7% yield) HCl salt as a white solid; ¹H NMR (300 MHz, DMSO-d₆) δ 13.23 (s, 1H, D₂O exchangeable), 12.09 (s, 1H, DO exchangeable), 9.05 (s, 3H, D₂O exchangeable), 8.97 (s, 1H), 8.41 (d, J=8.4 Hz, 1H), 8.35 (d, 1H), 8.10-7.96 (m, 3H), 7.69 (d, J=6.7 Hz, 1H), 7.31-7.24 (m, 3H), 7.21 (d, J=7.4 Hz, 1H), 6.99-6.88 (m, 1H), 5.49 (s, 2H), 4.78 (d, J=7.2 Hz, 2H), 4.09-3.95 (m, 2H), 3.95-3.78 (m, 2H), 3.53 (s, 2H), 3.33-3.30 (m, 1H); MS (ES+): 494.2 (M+1); (ES−): 492.2 (M−1).

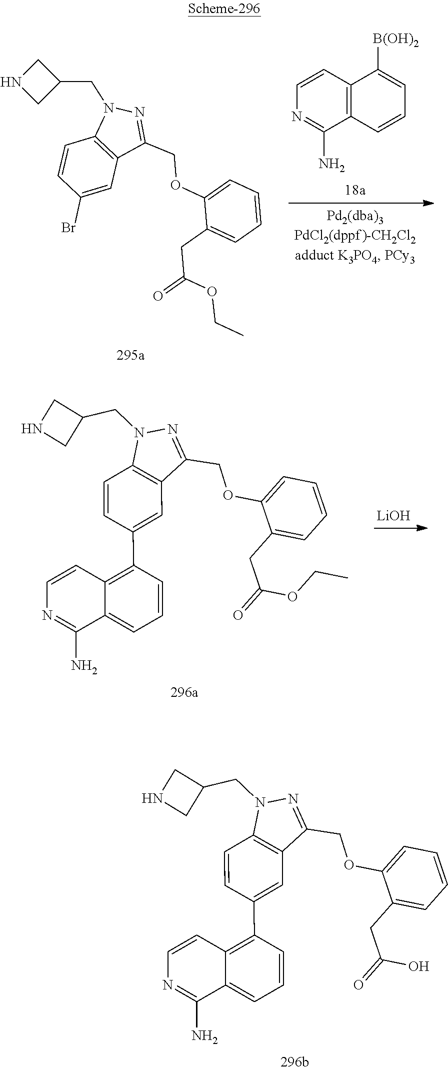 [SMILES: CCOC(=O)Cc1ccccc1OCc1nn(CC2CNC2)c2ccc(Br)cc12 ; Nc1nccc2c(B(O)O)cccc12 ; CCOC(=O)Cc1ccccc1OCc1nn(CC2CNC2)c2ccc(-c3cccc4c(N)nccc34)cc12 ; "LiOH" ; Nc1nccc2c(-c3ccc4c(c3)c(COc3ccccc3CC(=O)O)nn4CC3CNC3)cccc12]

### Preparation of 2-(2-((5-(1-aminoisoquinolin-5-yl)-1-(azetidin-3-ylmethyl)-1H-indazol-3-yl)methoxy)phenyl)acetic acid (296b)

### Step-1: Preparation of ethyl 2-(2-((5-(1-aminoisoquinolin-5-yl)-1-(azetidin-3-ylmethyl)-1H-indazol-3-yl)methoxy)phenyl)acetate (296a)

Compound 296a was prepared according to the procedure reported in step-2 of scheme-18, from ethyl 2-(2-((1-(azetidin-3-ylmethyl)-5-bromo-1H-indazol-3-yl)methoxy)phenyl)acetate (295a) (300 mg, 0.655 mmol) in dioxane/MeTHF (15 mL, Ratio: 1:1) using (1-aminoisoquinolin-5-yl)boronic acid (18a) (160 mg, 0.851 mmol), aqueous tripotassium phosphate (2M, 556 mg, 2.62 mmol), tricyclohexylphosphine (36.7 mg, 0.131 mmol), Pd₂(dba)₃ (59.9 mg, 0.065 mmol) and PdCl₂(dppf)-CH₂Cl₂Adduct (53.4 mg, 0.065 mmol) to afford after workup and purification using Method-BL, ethyl 2-(2-((5-(1-aminoisoquinolin-5-yl)-1-(azetidin-3-ylmethyl)-1H-indazol-3-yl)methoxy)phenyl)acetate (296a) (175 mg, 51% yield) as a yellow solid; MS (ES+): 522.2 (M+1).

### Step-2: Preparation of 2-(2-((5-(1-aminoisoquinolin-5-yl)-1-(azetidin-3-ylmethyl)-1H-indazol-3-yl)methoxy)phenyl)acetic acid (296b)

Compound 296b was prepared according to the procedure reported in step-2 of scheme-1, from ethyl 2-(2-((5-(1-aminoisoquinolin-5-yl)-1-(azetidin-3-ylmethyl)-1H-indazol-3-yl)methoxy)phenyl)acetate (296a) (115 mg, 0.220 mmol) in THF (3 mL) using lithium hydroxide hydrate (27.8 mg, 0.661 mmol) in water (1 mL) to afford after work up and purification using Method-B, 2-(2-((5-(1-aminoisoquinolin-5-yl)-1-(azetidin-3-ylmethyl)-1H-indazol-3-yl)methoxy)phenyl)acetic acid (296b) (15.8 mg, 15% yield) HCl salt as a white solid; ¹H NMR (300 MHz, DMSO-d₆) δ 13.47 (s, 1H, D₂O exchangeable), 12.06 (s, 1H, D₂O exchangeable), 9.29 (s, 2H, D₂O exchangeable), 9.13 (s, 1H, DO exchangeable), 8.65 (d, J=8.3 Hz, 1H), 8.01-7.79 (m, 4H), 7.64 (d, J=7.2 Hz, 1H), 7.53 (d, J=8.7 Hz, 1H), 7.24 (d, J=4.2 Hz, 2H), 7.17 (d, J=7.4 Hz, 1H), 6.96 (d, J=7.2 Hz, 1H), 6.93-6.83 (m, 1H), 5.44 (s, 2H), 4.82 (d, J=7.3 Hz, 2H), 4.11-3.94 (m, 2H), 3.93-3.77 (m, 2H), 3.47 (s, 2H), 3.30-3.25 (m, 1H); MS (ES+): 494.2 (M+1); (ES−): 492.1 (M−1).

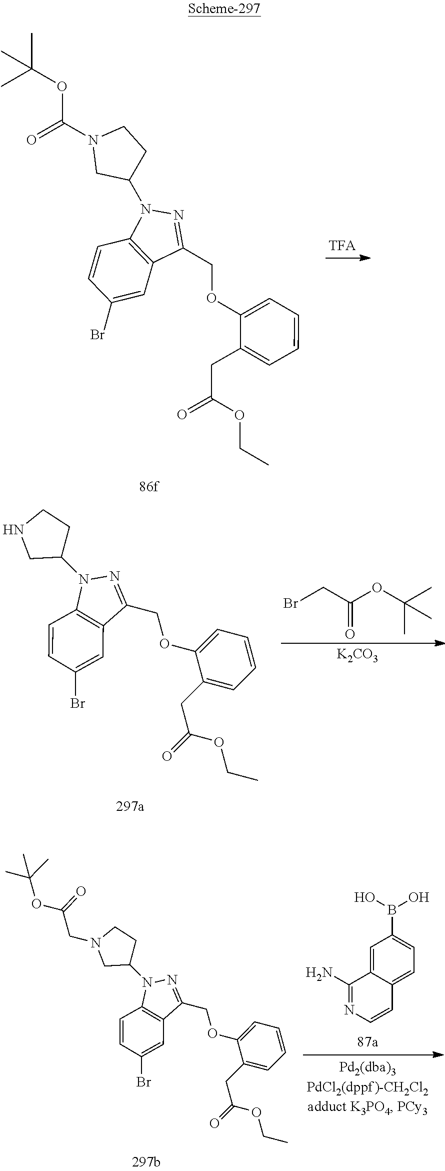

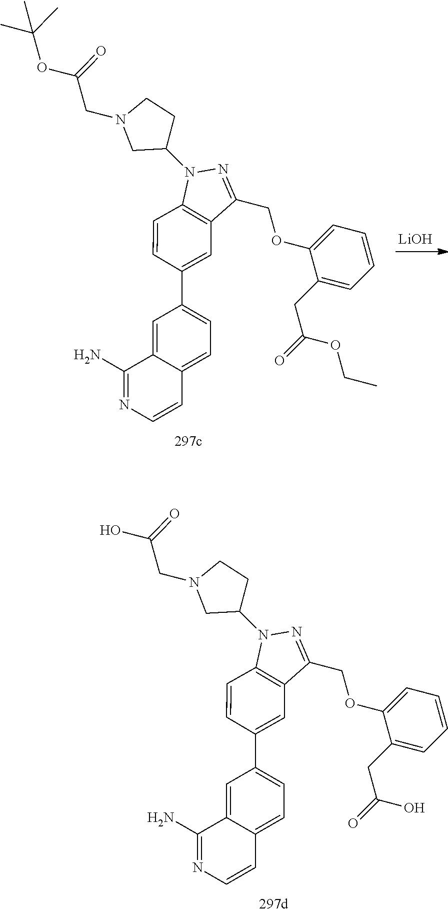

### Preparation of 2-(2-((5-(1-aminoisoquinolin-7-yl)-1-(1-(carboxymethyl)pyrrolidin-3-yl)-1H-indazol-3-yl)methoxy)phenyl)acetic acid (297d)

### Step-1: Preparation of ethyl 2-(2-((5-bromo-1-(pyrrolidin-3-yl)-1H-indazol-3-yl)methoxy)phenyl)acetate (297a)

Compound 297a was prepared according to the procedure reported in step-4 of scheme-9, from tert-butyl 3-(5-bromo-3-((2-(2-ethoxy-2-oxoethyl)phenoxy)methyl)-1H-indazol-1-yl)pyrrolidine-1-carboxylate (86f) (7.98 g, 14.29 mmol) in DCM (150 mL) using TFA (11.01 mL, 143 mmol) and stirring at RT for 16 h to afford after work up ethyl 2-(2-((5-bromo-1-(pyrrolidin-3-yl)-1H-indazol-3-yl)methoxy)phenyl)acetate (297a) (5.277 g, 81% yield) which was used as such for the next step; MS (ES+): 458.1 (M+1).

### Step-2: Preparation of tert-butyl 2-(3-(5-bromo-3-((2-(2-ethoxy-2-oxoethyl)phenoxy)methyl)-1H-indazol-1-yl)pyrrolidin-1-yl)acetate (297b)

To a solution of ethyl 2-(2-((5-bromo-1-(pyrrolidin-3-yl)-1H-indazol-3-yl)methoxy)phenyl)acetate (297a) (1 g, 2.182 mmol) in MeCN (10 mL) was added at RT tert-butyl 2-bromoacetate (0.511 g, 2.62 mmol) and K₂CO₃ (0.754 g, 5.45 mmol) and heated overnight at 70° C. The solid was removed via filtration and the filtrate was concentrated in vacuo. The residue obtained was purified using method-P, to give tert-butyl 2-(3-(5-bromo-3-((2-(2-ethoxy-2-oxoethyl)phenoxy)methyl)-1H-indazol-1-yl)pyrrolidin-1-yl)acetate (297b) (860 mg, 69% yield); MS (ES+): 572.2 (M+1).

### Step-3: Preparation of tert-butyl 2-(3-(5-(1-aminoisoquinolin-7-yl)-3-((2-(2-ethoxy-2-oxoethyl)phenoxy)methyl)-1H-indazol-1-yl)pyrrolidin-1-yl)acetate (297c)

Compound 297c was prepared according to the procedure reported in step-2 of scheme-18, from tert-butyl 2-(3-(5-bromo-3-((2-(2-ethoxy-2-oxoethyl)phenoxy)methyl)-1H-indazol-1-yl)pyrrolidin-1-yl)acetate (297b) (321 mg, 0.561 mmol) in dioxane/MeTHF (10 mL, Ratio: 1:1) using (1-aminoisoquinolin-7-yl)boronic acid (87a) (137 mg, 0.729 mmol), aqueous tripotassium phosphate (4 M, 0.561 mL, 2.243 mmol), tricyclohexylphosphine (31.4 mg, 0.112 mmol) Pd₂(dba)₃ (51.3 mg, 0.056 mmol) and PdCl₂(dppf)-CH₂Cl₂Adduct (45.8 mg, 0.056 mmol) to afford after workup and purification using method-AE, tert-butyl 2-(3-(5-(1-aminoisoquinolin-7-yl)-3-((2-(2-ethoxy-2-oxoethyl)phenoxy)methyl)-1H-indazol-1-yl)pyrrolidin-1-yl)acetate (297c) (127 mg, 36% yield) as a yellow solid; MS (ES+): 636.3 (M+1).

### Step-4: Preparation of 2-(2-((5-(1-aminoisoquinolin-7-yl)-1-(1-(carboxymethyl)pyrrolidin-3-yl)-1H-indazol-3-yl)methoxy)phenyl)acetic acid (297d)

Compound 297d was prepared according to the procedure reported in step-2 of scheme-1, from tert-butyl 2-(3-(5-(1-aminoisoquinolin-7-yl)-3-((2-(2-ethoxy-2-oxoethyl)phenoxy)methyl)-1H-indazol-1-yl)pyrrolidin-1-yl)acetate (297c) (127 mg, 0.2 mmol) in THF (3 mL) using lithium hydroxide hydrate (25.1 mg, 0.599 mmol) in water (1 mL) to afford after work up and purification using method-M, 2-(2-((5-(1-aminoisoquinolin-7-yl)-1-(1-(carboxymethyl)pyrrolidin-3-yl)-1H-indazol-3-yl)methoxy)phenyl)acetic acid (297d) (16.7 mg, 15% yield) HCl salt as a white solid; ¹H NMR (300 MHz, DMSO-d₆) δ 13.42 (s, 1H, D₂O Exchangeable), 9.27 (s, 3H, D₂O Exchangeable), 9.03 (s, 1H), 8.47-8.33 (m, 2H), 8.15-8.01 (m, 2H), 7.95 (d, J=8.9 Hz, 1H), 7.70 (d, J=6.9 Hz, 1H), 7.34-7.24 (m, 3H), 7.21 (d, J=7.4 Hz, 1H), 6.93 (t, J=6.8 Hz, 1H), 5.78 (s, 1H), 5.54 (s, 2H), 4.37 (s, 2H), 3.54 (s, 2H), 4.01-3.34 (m, 4H), 2.78-2.52 (m, 1H), 2.38-2.32 (m, 1H); MS (ES+): 552.2 (M+1); (ES−): 550.1 (M−1).

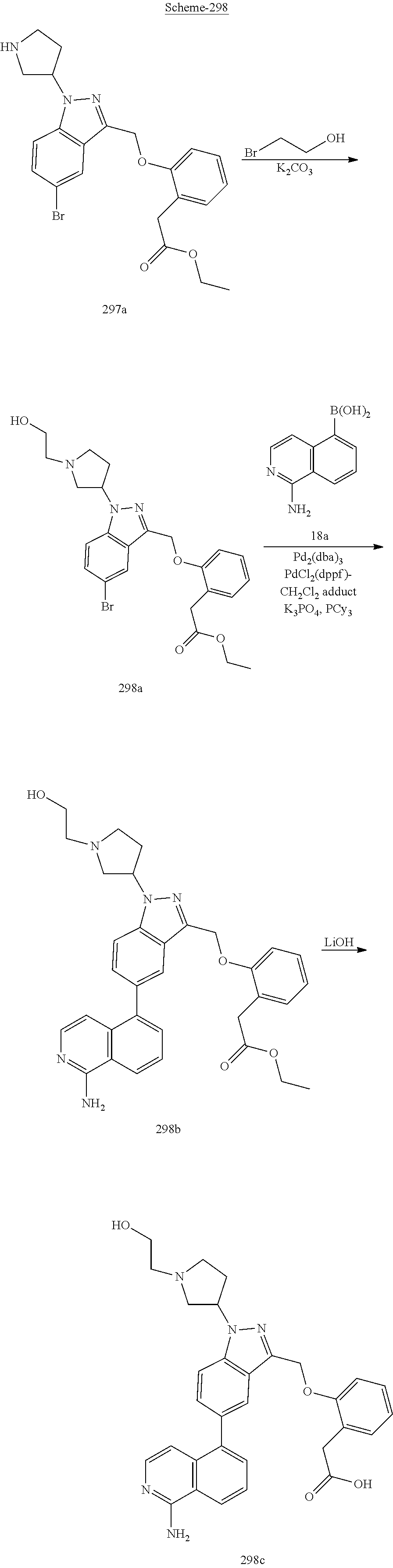

### Preparation of 2-(2-((5-(1-aminoisoquinolin-5-yl)-1-(1-(2-hydroxyethyl)pyrrolidin-3-yl)-1H-indazol-3-yl)methoxy)phenyl)acetic acid (298c)

### Step-1: Preparation of ethyl 2-(2-((5-bromo-1-(1-(2-hydroxyethyl)pyrrolidin-3-yl)-1H-indazol-3-yl)methoxy)phenyl)acetate (298a)

Compound 298a was prepared according to the procedure reported in step-2 of scheme-297, from ethyl 2-(2-((5-bromo-1-(pyrrolidin-3-yl)-1H-indazol-3-yl)methoxy)phenyl)acetate (297a) (1 g, 2.182 mmol) in MeCN (10 mL) using 2-bromoethanol (0.327 g, 2.62 mmol) and K₂CO₃ (0.754 g, 5.45 mmol) to afford after work up and purification using method-P, ethyl 2-(2-((5-bromo-1-(1-(2-hydroxyethyl)pyrrolidin-3-yl)-1H-indazol-3-yl)methoxy)phenyl)acetate (298a) (748 mg, 68% yield); MS (ES+): 502.2 (M+1).

### Step-2: Preparation of ethyl 2-(2-((5-(1-aminoisoquinolin-5-yl)-1-(1-(2-hydroxyethyl)pyrrolidin-3-yl)-1H-indazol-3-yl)methoxy)phenyl)acetate (298b)

Compound 298b was prepared according to the procedure reported in step-2 of scheme-18, from ethyl 2-(2-((5-bromo-1-(1-(2-hydroxyethyl)pyrrolidin-3-yl)-1H-indazol-3-yl)methoxy)phenyl)acetate (298a) (348 mg, 0.693 mmol) in dioxane/MeTHF (10 mL, Ratio: 1:1) using (1-aminoisoquinolin-5-yl)boronic acid (18a) (169 mg, 0.9 mmol), aqueous tripotassium phosphate (2 M, 588 mg, 2.77 mmol), tricyclohexylphosphine (38.8 mg, 0.139 mmol) Pd₂(dba)₃ (63.4 mg, 0.069 mmol) and PdCl₂(dppf)-CH₂Cl₂Adduct (56.6 mg, 0.069 mmol) to afford after workup and purification using method-AE, ethyl 2-(2-((5-(1-aminoisoquinolin-5-yl)-1-(1-(2-hydroxyethyl)pyrrolidin-3-yl)-1H-indazol-3-yl)methoxy)phenyl)acetate (298b) (137 mg, 35% yield) as a yellow solid; MS (ES+): 566.3 (M+1).

### Step-3: Preparation of 2-(2-((5-(1-aminoisoquinolin-5-yl)-1-(1-(2-hydroxyethyl)pyrrolidin-3-yl)-1H-indazol-3-yl)methoxy)phenyl)acetic acid (298c)

Compound 298c was prepared according to the procedure reported in step-2 of scheme-1, from ethyl 2-(2-((5-(1-aminoisoquinolin-5-yl)-1-(1-(2-hydroxyethyl)pyrrolidin-3-yl)-1H-indazol-3-yl)methoxy)phenyl)acetate (298b) (132 mg, 0.233 mmol) in THF (3 mL) using lithium hydroxide hydrate (29.4 mg, 0.700 mmol) in water (1 mL) to afford after work up and purification using method-M, 2-(2-((5-(1-aminoisoquinolin-5-yl)-1-(1-(2-hydroxyethyl)pyrrolidin-3-yl)-1H-indazol-3-yl)methoxy)phenyl)acetic acid (298c) (69.2 mg, 55% yield) HCl salt as a white solid; ¹H NMR (300 MHz, DMSO-d₆) δ 13.64 (s, 1H, D₂O Exchangeable), 12.07 (s, 1H, D₂O Exchangeable), 11.12 (s, 1H, D₂O Exchangeable), 9.36 (s, 2H, D₂O Exchangeable), 8.69 (d, J=8.3 Hz, 1H), 8.01-7.90 (m, 3H), 7.86 (t, J=7.8 Hz, 1H), 7.65 (d, J=7.2 Hz, 1H), 7.55 (d, J=8.8 Hz, 1H), 7.25 (d, J=4.3 Hz, 2H), 7.18 (d, J=7.4 Hz, 1H), 6.98-6.86 (m, 2H), 5.91-5.68 (m, 1H), 5.49 (s, 2H), 4.28-4.11 (m, 1H), 4.06-3.86 (m, 1H), 3.87-3.72 (m, 2H), 3.73-3.60 (m, 1H), 3.49 (s, 2H), 3.40-3.31 (m, 3H), 2.79-2.53 (m, 1H), 2.35 (m, 1H); MS (ES+): 538.2 (M+1); (ES−): 536.2 (M−1).

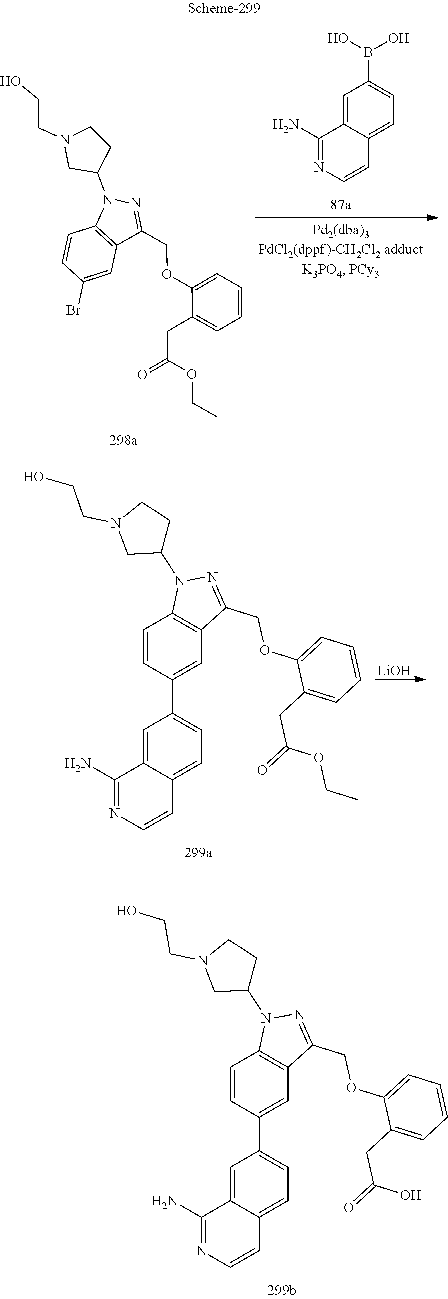

### Preparation of 2-(2-((5-(1-aminoisoquinolin-7-yl)-1-(1-(2-hydroxyethyl)pyrrolidin-3-yl)-1H-indazol-3-yl)methoxy)phenyl)acetic acid (299b)

### Step-1: Preparation of ethyl 2-(2-((5-(1-aminoisoquinolin-7-yl)-1-(1-(2-hydroxyethyl)pyrrolidin-3-yl)-1H-indazol-3-yl)methoxy)phenyl)acetate (299a)

Compound 299a was prepared according to the procedure reported in step-2 of scheme-18, from ethyl 2-(2-((5-bromo-1-(1-(2-hydroxyethyl)pyrrolidin-3-yl)-1H-indazol-3-yl)methoxy)phenyl)acetate (298a) (348 mg, 0.693 mmol) in dioxane/MeTHF (10 mL, Ratio: 1:1) using (1-aminoisoquinolin-7-yl)boronic acid (87a) (169 mg, 0.9 mmol), tripotassium phosphate (4 M aqueous, 693 μl, 2.77 mmol), tricyclohexylphosphine (38.8 mg, 0.139 mmol), Pd₂(dba)₃ (63 mg, 0.069 mmol) and PdCl₂(dppf)-CH₂Cl₂Adduct (57 mg, 0.069 mmol) to afford after workup and purification using method-AE, ethyl 2-(2-((5-(1-aminoisoquinolin-7-yl)-1-(1-(2-hydroxyethyl)pyrrolidin-3-yl)-1H-indazol-3-yl)methoxy)phenyl)acetate (299a) (145 mg, 37% yield) as a yellow solid; MS (ES+): 566.3 (M+1).

### Step-3: Preparation of 2-(2-((5-(1-aminoisoquinolin-7-yl)-1-(1-(2-hydroxyethyl)pyrrolidin-3-yl)-1H-indazol-3-yl)methoxy)phenyl)acetic acid (299b)

Compound 299b was prepared according to the procedure reported in step-2 of scheme-1, from ethyl 2-(2-((5-(1-aminoisoquinolin-7-yl)-1-(1-(2-hydroxyethyl)pyrrolidin-3-yl)-1H-indazol-3-yl)methoxy)phenyl)acetate (299a) (145 mg, 0.256 mmol) in THF (3 mL) using lithium hydroxide hydrate (32.3 mg, 0.769 mmol) in water (1 mL) to afford after work up and purification using method-M, 2-(2-((5-(1-aminoisoquinolin-7-yl)-1-(1-(2-hydroxyethyl)pyrrolidin-3-yl)-1H-indazol-3-yl)methoxy)phenyl)acetic acid (299b) (31.9 mg, 23% yield) HCl salt as a white solid; H NMR (300 MHz, DMSO-d₆) δ 13.52 (s, 1H, D₂O exchangeable), 12.11 (s, 1H, D₂O exchangeable), 10.99 (s, 1H, D₂O exchangeable), 9.33 (s, 2H, D₂O exchangeable), 9.06 (s, 1H), 8.53-8.36 (m, 2H), 8.17-8.02 (m, 2H), 8.02-7.90 (m, 1H), 7.71 (d, J=6.9 Hz, 1H), 7.34-7.24 (m, 3H), 7.22 (d, 7.4 Hz, 1H), 6.98-6.89 (m, 1H), 5.90-5.70 (m, 1H), 5.55 (s, 2H), 4.29-4.12 (m, 1H), 4.03-3.85 (m, 1H), 3.85-3.73 (m, 2H), 3.54 (s, 2H), 3.48-3.33 (m, 4H), 2.80-2.62 (m, 1H), 2.40-2.26 (m, 1H); MS (ES+): 538.2 (M+1); (ES−): 536.2 (M−1).

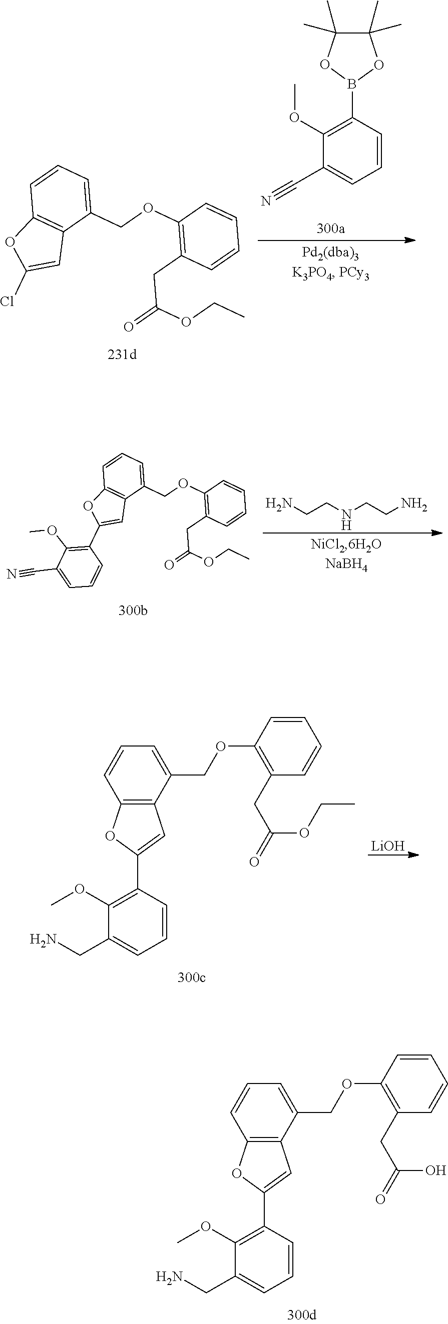

### Preparation of 2-(2-((2-(3-(aminomethyl)-2-methoxyphenyl)benzofuran-4-yl)methoxy)phenyl)acetic acid (300d)

### Step-1: Preparation of ethyl 2-(2-((2-(3-cyano-2-methoxyphenyl)benzofuran-4-yl)methoxy)phenyl)acetate (300b)

Compound 300b was prepared according to the procedure reported in step-3 of scheme-112, from ethyl 2-(2-((2-chlorobenzofuran-4-yl)methoxy)phenyl)acetate (231d) (150 mg, 0.435 mmol) in dioxane (6 mL) and 2Me-THF (3 mL) using 2-methoxy-3-(4,4,5,5-tetramethyl-1,3,2-dioxaborolan-2-yl)benzonitrile (300a) (322 mg, 0.870 mmol; CAS #874472-84-1), Pd₂(dba)₃ (80 mg, 0.087 mmol), aqueous tripotassium phosphate (4M, 0.435 mL, 1.740 mmol), tricyclohexylphosphine (48.8 mg, 0.174 mmol) and beating at 115° C. for 4.5 h. Solvent was removed in vacuo and residue was purified using method-V, ethyl 2-(2-((2-(3-cyano-2-methoxyphenyl)benzofuran-4-yl)methoxy)phenyl)acetate (300b) (162 mg, 84% yield) as a yellow semi-solid; MS (ES+): 464.1 (M+Na).

### Step-2: Preparation of ethyl 2-(2-((2-(3-(aminomethyl)-2-methoxyphenyl)benzofuran-4-yl)methoxy)phenyl)acetate (300c)

A solution of ethyl 2-(2-((2-(3-cyano-2-methoxyphenyl)benzofuran-4-yl)methoxy)phenyl)acetate (300b) (160 mg, 0.362 mmol) in anhydrous MeOH (10 mL) was added nickel(II) chloride hexahydrate (21.54 mg, 0.091 mmol) followed by sodium borohydride (200 mg, 5.29 mmol) in five portions in one hour interval. (Need to add excessive amount in many portions to make the reaction completed). The reaction mixture was then treated with N1-(2-aminoethyl)ethane-1,2-diamine (0.078 mL, 0.725 mmol) and was stirred for 10 min before the solvent was evaporated. The residue was purified using method-BM, to furnish ethyl 2-(2-((2-(3-(aminomethyl)-2-methoxyphenyl)benzofuran-4-yl)methoxy)phenyl)acetate (300c) (64 mg, 0.144 mmol, 39.6% yield) as a yellow oil; MS (ES+): 446.2 (M+1).

### Step-3: Preparation of 2-(2-((2-(3-(aminomethyl)-2-methoxyphenyl)benzofuran-4-yl)methoxy)phenyl)acetic acid (300d)

Compound 300d was prepared according to the procedure reported in step-2 of scheme-1, from ethyl 2-(2-((2-(3-(aminomethyl)-2-methoxyphenyl)benzofuran-4-yl)methoxy)phenyl)acetate (300c) (65 mg, 0.146 mmol) in MeOH/THF (3 mL cach) using a solution of lithium hydroxide monohydrate (50 mg, 1.192 mmol) in water (2 mL) and stirring at RT for 5 h. This gave after workup and purification using method-G, 2-(2-((2-(3-(aminomethyl)-2-methoxyphenyl)benzofuran-4-yl)methoxy)phenyl)acetic acid (300d) (49 mg, 80% yield) HCl salt as a white solid; JH NMR (300 MHz, DMSO-d₆) δ 12.19 (s, 1H), 8.58 (s, 3H), 7.98 (dd, J=7.9, 1.7 Hz, 1H), 7.69-7.59 (m, 2H), 7.57 (s, 1H), 7.45-7.33 (m, 3H), 7.29-7.15 (m, 3H), 6.92 (t, J=7.2 Hz, 1H), 5.44 (s, 2H), 4.15 (q, J=5.8 Hz, 2H), 3.77 (s, 3H), 3.59 (s, 2H); MS (ES+): 418.1 (M+1); (ES−): 416.1 (M−1).

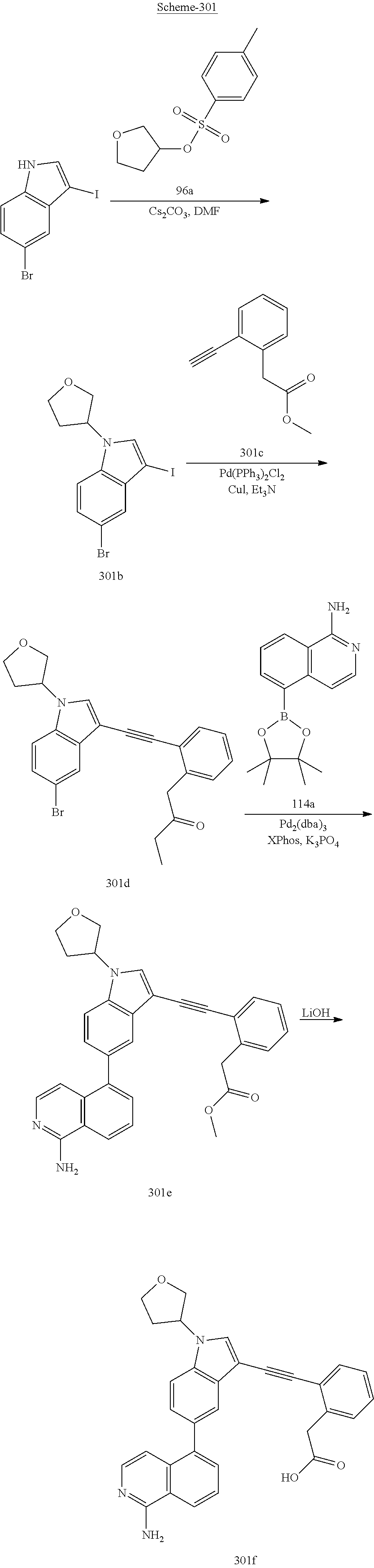

### Preparation of 2-(2-((5-(1-aminoisoquinolin-5-yl)-1-(tetrahydrofuran-3-yl)-1H-indol-3-yl)ethynyl)phenyl)acetic acid (301f)

### Step-1: Preparation of 2-(2-((5-(1-aminoisoquinolin-5-yl)-1-(tetrahydrofuran-3-yl)-1H-indol-3-yl)ethynyl)phenyl)acetic acid (301b)

Compound 301b Prepared according to the procedure reported in step-2 of scheme-86, from 5-bromo-3-iodo-1H-indole (301a) (2.1 g, 6.52 mmol; CAS #868694-19-3) in DMF (35 mL) using tetrahydrofuran-3-yl 4-methylbenzenesulfonate (96a) (1.897 g, 7.83 mmol), Cs₂CO₃ (4.25 g, 13.05 mmol) and heating at 90° C. for 22 h to afford after work up and purification using method-O, 2-(2-((5-(1-aminoisoquinolin-5-yl)-1-(tetrahydrofuran-3-yl)-1H-indol-3-yl)ethynyl)phenyl)acetic acid (301b) (1.9 g, 74.3% yield) as a yellow oil; ¹H NMR (300 MHz, DMSO-d₆) δ7.66 (s, 1H), 7.61 (d, J=8.7 Hz, 1H), 7.42 (d, J=2.0 Hz, 1H), 7.36 (dd, J=8.8, 1.9 Hz, 1H), 5.33-5.21 (m, 1H), 4.16-4.03 (m, 1H), 3.97-3.86 (m, 2H), 3.86-3.72 (m, 1H), 2.49-2.41 (m, 1H), 2.19-2.04 (m, 1H).

### Step-2: Preparation of methyl 2-(2-((5-bromo-1-(tetrahydrofuran-3-yl)-1H-indol-3-yl)ethynyl)phenyl)acetate (301d)

To a degassed mixture of copper (I) iodide (24.29 mg, 0.128 mmol) and methyl 2-(2-ethynylphenyl)acetate (301c) (222 mg, 1.275 mmol; CAS #637348-19-7) in anhydrous THF (20 mL) was added Pd(PPh₃)₂Cl₂ (90 mg, 0.128 mmol) under Ar stirred for 5 min, followed by sequential addition of triethylamine (1778 μl, 12.75 mmol) and 5-bromo-3-iodo-1-(tetrahydrofuran-3-yl)-1H-indole (301b) (500 mg, 1.275 mmol). The reaction mixture was stirred at RT for 5 h, filtered through a pad of Celite and the filtrate was concentrated in vacuo. The crude oil was poured into water and was extracted with DCM. Combined organics were washed with brine, dried, filtered and concentrated to dryness in vacuo. The residue obtained was purified using method-O, to give methyl 2-(2-((5-bromo-1-(tetrahydrofuran-3-yl)-1H-indol-3-yl)ethynyl)phenyl)acetate (301d) (233 mg, 42% yield) as yellow oil; ¹H NMR (300 MHz, DMSO-d₆) ¿ 7.89-7.80 (m, 2H), 7.68 (d, J=8.8 Hz, 1H), 7.58-7.52 (m, 1H), 7.50-7.32 (m, 4H), 5.35-5.24 (m, 1H), 4.11 (q, J=7.7 Hz, 1H), 4.00-3.91 (m, 4H), 3.89-3.77 (m, 1H), 3.65 (s, 3H), 2.58-2.52 (m, 1H), 2.25-2.11 (m, 1H).

### Step-3: Preparation of methyl 2-(2-((5-(1-aminoisoquinolin-5-yl)-1-(tetrahydrofuran-3-yl)-1H-indol-3-yl)ethynyl)phenyl)acetate (301e)

Compound 301e was prepared according to the procedure and method of purification reported in step-3 of scheme-12, from methyl 2-(2-((5-bromo-1-(tetrahydrofuran-3-yl)-1H-indol-3-ylethynyl)phenyl)acetate (301d) (230 mg, 0.525 mmol) in dioxane/2-MeTHF (4 mL, Ratio: 3:1) using 5-(4,4,5,5-tetramethyl-1,3,2-dioxaborolan-2-yl)isoquinolin-1-amine (114a) (283 mg, 1.049 mmol), tripotassium phosphate (223 mg, 1.049 mmol), XPhos (25.02 mg, 0.052 mmol) and Pd₂(dba)₃ (96 mg, 0.105 mmol) to afford after work up and purification using method-U, methyl 2-(2-((5-(1-aminoisoquinolin-5-yl)-1-(tetrahydrofuran-3-yl)-1H-indol-3-yl)ethynyl)phenyl)acetate (301e) (152 mg, 58% yield) as a clear oil; MS (ES+): 502.2 (M+1).

### Step-4: Preparation of 2-(2-((5-(1-aminoisoquinolin-5-yl)-1-(tetrahydrofuran-3-yl)-1H-indol-3-yl)ethynyl)phenyl)acetic acid (301f)

Compound 301f was prepared according to the procedure and method of purification reported in step-2 of scheme-1, from methyl 2-(2-((5-(1-aminoisoquinolin-5-yl)-1-(tetrahydrofuran-3-yl)-1H-indol-3-yl)ethynyl)phenyl)acetate (301e) (152 mg, 0.303 mmol) in THE (3 mL) using lithium hydroxide (37.7 mg, 1.574 mmol) in water (1 mL) to afford after work up and purification using method-M, 2-(2-((5-(1-aminoisoquinolin-5-yl)-1-(tetrahydrofuran-3-yl)-1H-indol-3-yl)ethynyl)phenyl)acetic acid (3011) (21 mg, 8% yield) HCl salt as a white solid; H NMR (300 MHz, DMSO-d₆) δ 13.26 (s, 1H, D₂O exchangeable), 12.30 (s, 1H, D₂O exchangeable), 9.17 (s, 2H, D₂O exchangeable), 8.62 (d, J=8.4 Hz, 1H), 7.99 (d, J=7.2 Hz, 1H), 7.94 (s, 1H), 7.90-7.82 (m, 2H), 7.79 (s, 1H), 7.64 (d, J=7.3 Hz, 1H), 7.55-7.45 (m, 1H), 7.38-7.24 (m, 4H), 7.05 (d, J=7.2 Hz, 1H), 5.48-5.34 (m, 1H), 4.21-4.11 (m, 1H), 4.02 (d, J=4.4 Hz, 2H), 3.92-3.85 (m, 1H), 3.81 (s, 2H), 2.65-2.53 (m, 1H), 2.32-2.18 (m, 1H); MS (ES+): 488.2 (M+1).

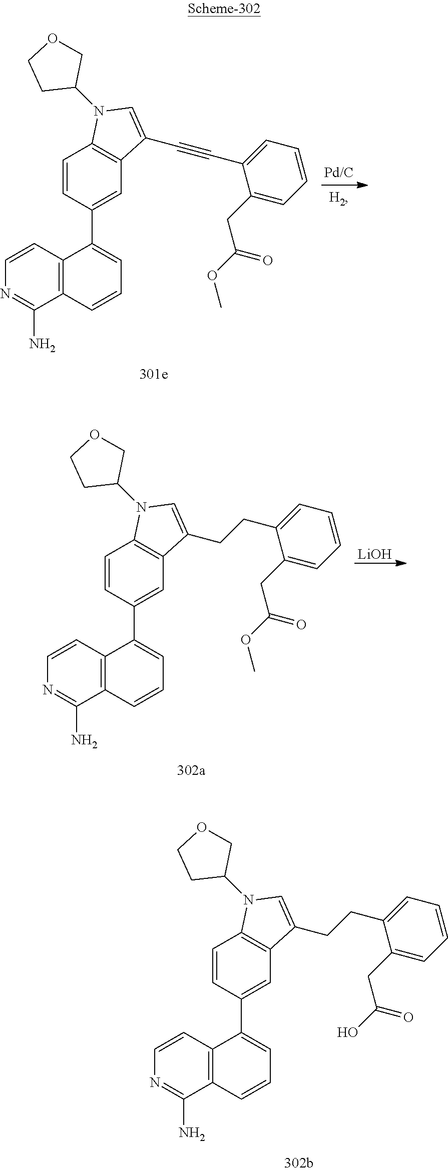

### Preparation of 2-(2-(2-(5-(1-aminoisoquinolin-5-yl)-1-(tetrahydrofuran-3-yl)-1H-indol-3-yl)ethyl)phenyl)acetic acid (302b)

### Step-1: Preparation of methyl 2-(2-(2-(5-(1-aminoisoquinolin-5-yl)-1-(tetrahydrofuran-3-yl)-1H-indol-3-yl)ethyl)phenyl)acetate (302a)

To a degassed solution of methyl 2-(2-((5-(1-aminoisoquinolin-5-yl)-1-(tetrahydrofuran-3-yl)-1H-indol-3-yl)ethynyl)phenyl)acetate (301e) (100 mg, 0.199 mmol) in MeOH (10 mL) was added Pd/C (42.4 mg, 0.04 mmol). The mixture was degassed and filled with H₂ balloon and stirred for 30 min at RT. The reaction mixture was diluted with EtOAc and filtered through a pad of Celite. The solvent was removed to afford methyl 2-(2-(2-(5-(1-aminoisoquinolin-5-yl)-1-(tetrahydrofuran-3-yl)-1H-indol-3-yl)ethyl)phenyl)acetate (302a) (13 mg, 13% yield) which was used as such for the next step; MS (ES+): 506.2 (M+1).

### Step-2: Preparation of 2-(2-(2-(5-(1-aminoisoquinolin-5-yl)-1-(tetrahydrofuran-3-yl)-1H-indol-3-yl)ethyl)phenyl)acetic acid (302b)

Compound 302b was prepared according to the procedure and method of purification reported in step-2 of scheme-1, from methyl 2-(2-(2-(5-(1-aminoisoquinolin-5-yl)-1-(tetrahydrofuran-3-yl)-1H-indol-3-yl)ethyl)phenyl)acetate (302a) (13 mg, 0.026 mmol) in THF/MeOH (6 mL) using lithium hydroxide (14.32 mg, 0.598 mmol) in water (1 mL) to afford after work up and purification using method-M, 2-(2-(2-(5-(1-aminoisoquinolin-5-yl)-1-(tetrahydrofuran-3-yl)-1H-indol-3-yl)ethyl)phenyl)acetic acid (302b) (2.4 mg, 2% yield) HCl salt as a white solid; ¹H NMR (300 MHz, DMSO-d₆) δ 13.10 (s, 1H, D₂O exchangeable), 12.28 (s, 1H, D₂O exchangeable), 9.04 (s, 2H, D₂O exchangeable), 8.57 (d, J=8.2 Hz, 1H), 7.97-7.90 (m, 1H), 7.85 (t, J=7.8 Hz, 1H), 7.70 (d, J=8.5 Hz, 1H), 7.67-7.59 (m, 2H), 7.32 (s, 1H), 7.27-7.11 (m, 5H), 7.08 (d, J==7.2 Hz, 1H), 5.33-5.24 (m, 1H), 4.13-3.98 (m, 2H), 3.95-3.82 (m, 2H), 3.57 (s, 2H), 2.94 (s, 4H), 2.58-2.54 (m, 1H), 2.18-2.03 (m, 1H); MS (ES+): 492.3 (M+1); (ES−): 490.1 (M−1).

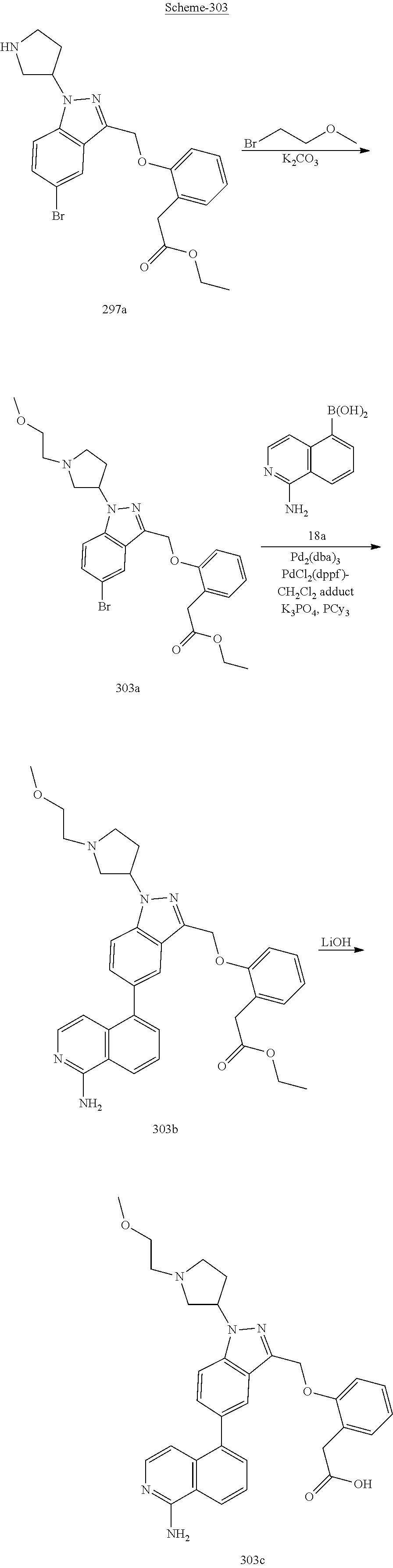

### Preparation of 2-(2-((5-(1-aminoisoquinolin-5-yl)-1-(1-(2-methoxyethyl)pyrrolidin-3-yl)-1H-indazol-3-yl)methoxy)phenyl)acetic acid (303c)

### Step-1: Preparation of ethyl 2-(2-((5-bromo-1-(1-(2-methoxyethyl)pyrrolidin-3-yl)-1H-indazol-3-yl)methoxy)phenyl)acetate (303a)

Compound 303a was prepared according to the procedure reported in step-2 of scheme-297, from ethyl 2-(2-((5-bromo-1-(pyrrolidin-3-yl)-1H-indazol-3-yl)methoxy)phenyl)acetate (297a) (1 g, 2.182 mmol) in MeCN (10 mL) using 1-bromo-2-methoxyethane (0.364 g, 2.62 mmol) and K₂CO₃ (0.754 g, 5.45 mmol) to afford after work up and purification using method-P, ethyl 2-(2-((5-bromo-1-(1-(2-methoxyethyl)pyrrolidin-3-yl)-1H-indazol-3-yl)methoxy)phenyl)acetate (303a) (600 mg, 53% yield); MS (ES+): 516.1 (M+1).

### Step-2: Preparation of ethyl 2-(2-((5-(1-aminoisoquinolin-5-yl)-1-(1-(2-methoxyethyl)pyrrolidin-3-yl)-1H-indazol-3-yl)methoxy)phenyl)acetate (303b)

Compound 303b was prepared according to the procedure reported in step-2 of scheme-18, from ethyl 2-(2-((5-bromo-1-(1-(2-methoxyethyl)pyrrolidin-3-yl)-1H-indazol-3-yl)methoxy)phenyl)acetate (303a) (285 mg, 0.552 mmol) in dioxane/MeTHF (5 mL, Ratio: 1:1) using (1-aminoisoquinolin-5-yl)boronic acid (18a) (135 mg, 0.717 mmol), aqueous tripotassium phosphate (2 M, 469 mg, 2.207 mmol), tricyclohexylphosphine (31 mg, 0.110 mmol), Pd₂(dba)₃ (50.5 mg, 0.055 mmol) and PdCl₂(dppf)-CH₂Cl₂Adduct (45.1 mg, 0.055 mmol) to afford after workup and purification using method-AE, ethyl 2-(2-((5-(1-aminoisoquinolin-5-yl)-1-(1-(2-methoxyethyl)pyrrolidin-3-yl)-1H-indazol-3-yl)methoxy)phenyl)acetate (303b) (119 mg, 37% yield) as a yellow solid; MS (ES+): 580.3 (M+1).

### Step-3: Preparation of 2-(2-((5-(1-aminoisoquinolin-5-yl)-1-(1-(2-methoxyethyl)pyrrolidin-3-yl)-1H-indazol-3-yl)methoxy)phenyl)acetic acid (303c)

Compound 303c was prepared according to the procedure reported in step-2 of scheme-1, from ethyl 2-(2-((5-(1-aminoisoquinolin-5-yl)-1-(1-(2-methoxyethyl)pyrrolidin-3-yl)-1H-indazol-3-yl)methoxy)phenyl)acetate (303b) (119 mg, 0.205 mmol) in THF (3 mL) using lithium hydroxide hydrate (25.8 mg, 0.616 mmol) in water (1 mL) to afford after work up and purification using method-M, 2-(2-((5-(1-aminoisoquinolin-5-yl)-1-(1-(2-methoxyethyl)pyrrolidin-3-yl)-1H-indazol-3-yl)methoxy)phenyl)acetic acid (303c) (15.2 mg, 13% yield) HCl salt as a white solid; ¹H NMR (300 MHz, DMSO-d₆) δ 13.31 (s, 1H, D₂O exchangeable), 12.05 (s, 1H, D₂O exchangeable), 10.89 (s, 1H, D₂O exchangeable), 9.19 (s, 2H, D₂O exchangeable), 8.65 (d, J=8.3 Hz, 1H), 8.00-7.82 (m, 4H), 7.65 (d, J=7.3 Hz, 1H), 7.57 (d, J=8.7 Hz, 1H), 7.26 (d, J=4.2 Hz, 2H), 7.20 (d, J=7.4 Hz, 1H), 6.99-6.85 (m, 2H), 5.89-5.68 (m, 1H), 5.51 (s, 2H), 4.22-4.11 (m, 1H), 3.88-3.66 (m, 3H), 3.60-3.55 (m, 2H), 3.50 (s, 2H), 3.37-3.34 (m, 5H), 2.57-2.55 (m, 1H), 2.43-2.31 (m, 1H); MS (ES+): 552.3 (M+1); (ES−): 550.2 (M−1).

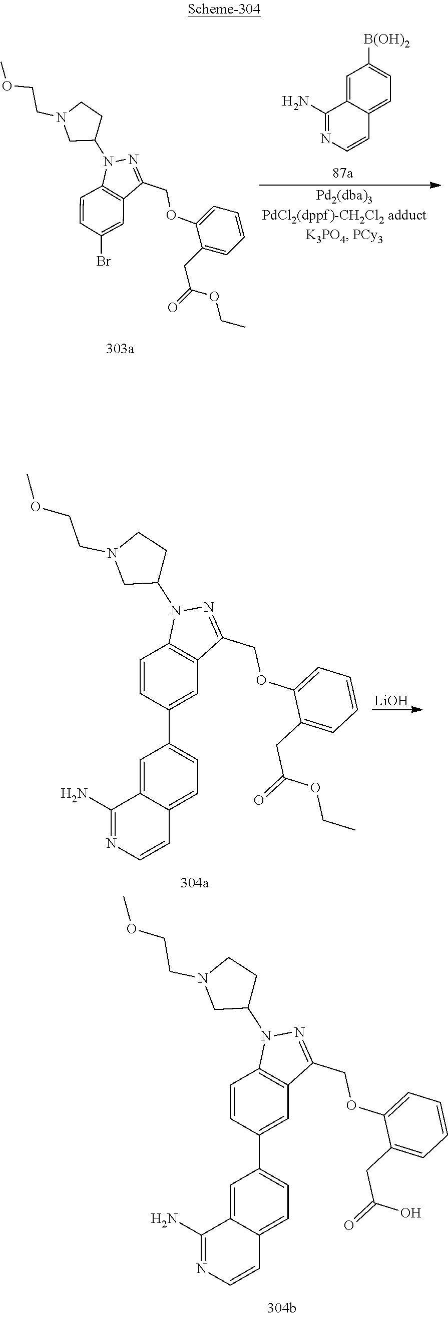

### Preparation of 2-(2-((5-(1-aminoisoquinolin-7-yl)˜1-(1-(2-methoxyethyl)pyrrolidin-3-yl)-1H-indazol-3-yl)methoxy)phenyl)acetic acid (304b)

### Step-1: Preparation of ethyl 2-(2-((5-(1-aminoisoquinolin-7-yl)-1-(1-(2-methoxyethyl)pyrrolidin-3-yl)-1H-indazol-3-yl)methoxy)phenyl)acetate (304a)

Compound 304a was prepared according to the procedure reported in step-2 of scheme-18, from ethyl 2-(2-((5-bromo-1-(1-(2-methoxyethyl)pyrrolidin-3-yl)-1H-indazol-3-yl)methoxy)phenyl)acetate (303a) (287 mg, 0.556 mmol) in dioxane/MeTHF (5 mL, Ratio: 1:1) using (1-aminoisoquinolin-7-yl)boronic acid (87a) (136 mg, 0.722 mmol), aqueous tripotassium phosphate (4 M, 556 μl, 2.223 mmol), tricyclohexylphosphine (31.2 mg, 0.111 mmol), Pd₂(dba)₃ (50.9 mg, 0.056 mmol) and PdCl₂(dppf)-CH₂Cl₂Adduct (45.4 mg, 0.056 mmol) to afford after workup and purification using method-AE, ethyl 2-(2-((5-(1-aminoisoquinolin-7-yl)-1-(1-(2-methoxyethyl)pyrrolidin-3-yl)-1H-indazol-3-yl)methoxy)phenyl)acetate (304a) (128 mg, 40% yield) as a yellow solid; MS (ES+): 580.3 (M+1).

### Step-3: Preparation of 2-(2-((5-(1-aminoisoquinolin-7-yl)-1-(1-(2-methoxyethyl)pyrrolidin-3-yl)-1H-indazol-3-yl)methoxy)phenyl)acetic acid (304b)

Compound 304b was prepared according to the procedure reported in step-2 of scheme-1, from ethyl 2-(2-((5-(1-aminoisoquinolin-7-yl)-1-(1-(2-methoxyethyl)pyrrolidin-3-yl)-1H-indazol-3-yl)methoxy)phenyl)acetate (304a) (127 mg, 0.219 mmol) in THF (3 mL) using lithium hydroxide hydrate (27.6 mg, 0.657 mmol) in water (1 mL) to afford after work up and purification using method-M, 2-(2-((5-(1-aminoisoquinolin-7-yl)-1-(1-(2-methoxyethyl)pyrrolidin-3-yl)-1H-indazol-3-yl)methoxy)phenyl)acetic acid (304b) (26.0 mg, 22% yield) HCl salt as a white solid; ¹H NMR (300 MHz, DMSO-d₆) δ 13.48 (s, 1H, D₂O) exchangeable), 12.14 (s, 1H, D₂O exchangeable), 11.20 (s, 1H, D₂O exchangeable), 9.32 (s, 2H, D₂O exchangeable), 9.05 (s, 1H), 8.49-8.37 (m, 2H), 8.18-8.00 (m, 2H), 8.00-7.90 (m, 1H), 7.70 (d, J=6.9 Hz, 1H), 7.31-7.24 (m, 3H), 7.21 (d, J=7.4 Hz, 1H), 6.98-6.89 (m, 1H), 5.82-5.70 (m, 1H), 5.54 (s, 2H), 4.24-4.10 (m, 1H), 3.97-3.82 (m, 2H), 3.80-3.68 (m, 2H), 3.53 (s, 3H), 3.34-3.29 (m, 5H), 2.79-2.55 (m, 1H), 2.38-2.24 (m, 1H); MS (ES+): 552.20 (M+1); (ES−): 550.1 (M−1).

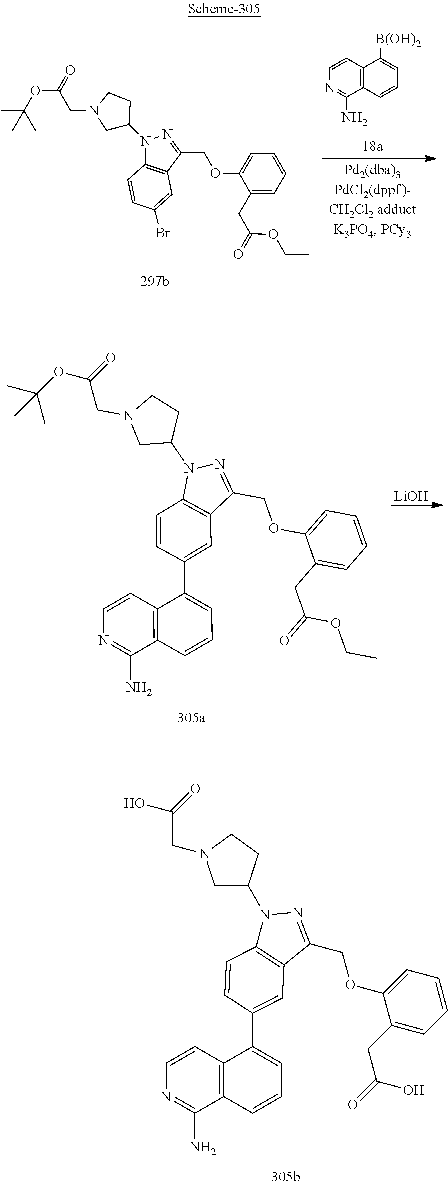

### Preparation of 2-(2-((5-(1-aminoisoquinolin-5-yl)-1-(1-(carboxymethyl)pyrrolidin-3-yl)-1H-indazol-3-yl)methoxy)phenyl)acetic acid (305b)

### Step-1: Preparation of tert-butyl 2-(3-(5-(1-aminoisoquinolin-5-yl)-3-((2-(2-ethoxy-2-oxoethyl)phenoxy)methyl)-1H-indazol-1-yl)pyrrolidin-1-yl)acetate (305a)

Compound 305a was prepared according to the procedure reported in step-2 of scheme-18, from tert-butyl 2-(3-(5-bromo-3-((2-(2-ethoxy-2-oxoethyl)phenoxy)methyl)-1H-indazol-1-yl)pyrrolidin-1-yl)acetate (297b) (321 mg, 0.561 mmol) in dioxane/MeTHF (5 mL, Ratio: 1:1) using (1-aminoisoquinolin-5-yl)boronic acid (18a) (137 mg, 0.729 mmol), aqueous tripotassium phosphate (2 M, 476 mg, 2.243 mmol), tricyclohexylphosphine (31.4 mg, 0.112 mmol) Pd₂(dba)₃ (51.3 mg, 0.056 mmol) and PdCl₂(dppf)-CH₂Cl₂Adduct (45.8 mg, 0.056 mmol) to afford after workup and purification using method-AE, tert-butyl 2-(3-(5-(1-aminoisoquinolin-5-yl)-3-((2-(2-ethoxy-2-oxoethyl)phenoxy)methyl)-1H-indazol-1-yl)pyrrolidin-1-yl)acetate (305a) (107 mg, 30% yield) as a yellow solid; MS (ES+): 636.3 (M+1).

### Step-2: Preparation of 2-(2-((5-(1-aminoisoquinolin-5-yl)-1-(1-(carboxymethyl)pyrrolidin-3-yl)-1H-indazol-3-yl)methoxy)phenyl)acetic acid (305b)

Compound 305b was prepared according to the procedure reported in step-2 of scheme-1, from tert-butyl 2-(3-(5-(1-aminoisoquinolin-5-yl)-3-((2-(2-ethoxy-2-oxoethyl)phenoxy)methyl)-1H-indazol-1-yl)pyrrolidin-1-yl)acetate (305a) (107 mg, 0.168 mmol) in THF (3 mL) using lithium hydroxide hydrate (21.19 mg, 0.505 mmol) in water (1 mL) to afford after work up and purification using method-M, 2-(2-((5-(1-aminoisoquinolin-5-yl)-1-(1-(carboxymethyl)pyrrolidin-3-yl)-1H-indazol-3-yl)methoxy)phenyl)acetic acid (305b) (24.2 mg, 26% yield) HCl salt as a white solid; ¹H NMR (300 MHz, DMSO-d₆) δ 9.12 (s, 2H, D₂O exchangeable), 8.62 (d, J=8.3 Hz, 1H), 7.99-7.90 (m, 3H), 7.85 (t, J=7.8 Hz, 1H), 7.63 (d, J=7.1 Hz, 1H), 7.54 (d, J=8.8 Hz, 1H), 7.29-7.22 (m, 2H), 7.18 (d, J=7.4 Hz, 1H), 6.98-6.86 (m, 2H), 5.80-5.65 (m, 1H), 5.49 (s, 2H), 4.22 (s, 2H), 4.04-3.87 (m, 1H), 3.86-3.64 (m, 1H), 3.66-3.51 (m, 2H), 3.48 (s, 2H), 2.75-2.57 (m, 1H), 2.44-2.23 (m, 1H); MS (ES+): 552.20 (M+1); (ES−): 550.2 (M−1).

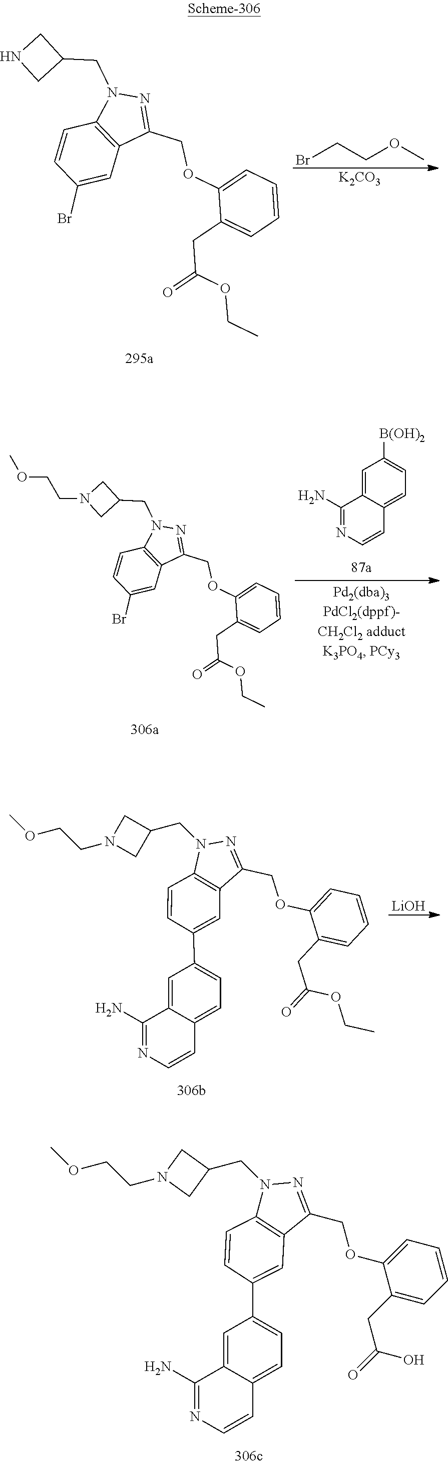

### Preparation of 2-(2-((5-(1-aminoisoquinolin-7-yl)-1-((1-(2-methoxyethyl)azetidin-3-yl)methyl)-1H-indazol-3-yl)methoxy)phenyl)acetic acid (306c)

### Step-1: Preparation of ethyl 2-(2-((5-bromo-1-((1-(2-methoxyethyl)azetidin-3-yl)methyl)-1H-indazol-3-yl)methoxy)phenyl)acetate (306a)

Compound 306a was prepared according to the procedure reported in step-2 of scheme-297, from ethyl 2-(2-((1-(azetidin-3-ylmethyl)-5-bromo-1H-indazol-3-yl)methoxy)phenyl)acetate (295a) (1 g, 2.182 mmol) in MeCN (15 mL) using 1-bromo-2-methoxyethane (0.364 g, 2.62 mmol) and K₂CO₃ (0.754 g, 5.45 mmol) to afford after work up and purification using method-P, ethyl 2-(2-((5-bromo-1-((1-(2-methoxyethyl)azetidin-3-yl)methyl)-1H-indazol-3-yl)methoxy)phenyl)acetate (306a) (387 mg, 34% yield); MS (ES+): 516.1 (M+1).

### Step-2: Preparation of ethyl 2-(2-((5-(1-aminoisoquinolin-7-yl)-1-((1-(2-methoxyethyl)azetidin-3-yl)methyl)-1H-indazol-3-yl)methoxy)phenyl)acetate (306b)

Compound 306b was prepared according to the procedure reported in step-2 of scheme-18, from ethyl 2-(2-((5-bromo-1-((1-(2-methoxyethyl)azetidin-3-yl)methyl)-1H-indazol-3-yl)methoxy)phenyl)acetate (306a) (275 mg, 0.532 mmol) in dioxane/MeTHF (5 mL, Ratio: 1:1) using (1-aminoisoquinolin-7-yl)boronic acid (87a) (130 mg, 0.692 mmol), aqueous tripotassium phosphate (4 M, 532 μL, 2.13 mmol), tricyclohexylphosphine (29.9 mg, 0.106 mmol), Pd₂(dba)₃ (48.8 mg, 0.053 mmol) and PdCl₂(dppf)-CH₂Cl₂Adduct (43.5 mg, 0.053 mmol) to afford after workup and purification using method-AE, ethyl 2-(2-((5-(1-aminoisoquinolin-7-yl)-1-((1-(2-methoxyethyl)azetidin-3-yl)methyl)-1H-indazol-3-yl)methoxy)phenyl)acetate (306b) (111 mg, 36% yield) as a yellow solid; MS (ES+): 580.30 (M+1).

### Step-3: Preparation of 2-(2-((5-(1-aminoisoquinolin-7-yl)-1-((1-(2-methoxyethyl)azetidin-3-yl)methyl)-1H-indazol-3-yl)methoxy)phenyl)acetic acid (306c)

Compound 306c was prepared according to the procedure reported in step-2 of scheme-1, from ethyl 2-(2-((5-(1-aminoisoquinolin-7-yl)-1-((1-(2-methoxyethyl)azetidin-3-yl)methyl)-1H-indazol-3-yl)methoxy)phenyl)acetate (306b) (110 mg, 0.190 mmol) in THF (3 mL) using lithium hydroxide hydrate (23.89 mg, 0.569 mmol) in water (1 mL) to afford after work up and purification using method-M, 2-(2-((5-(1-aminoisoquinolin-7-yl)-1-((1-(2-methoxyethyl)azetidin-3-yl)methyl)-1H-indazol-3-yl)methoxy)phenyl)acetic acid (306c) (38.0 mg, 36% yield) HCl salt as a white solid; ¹H NMR (300 MHz, DMSO-d₆) δ 9.04 (s, 2H, D₂O Exchangeable), 8.98 (s, 1H), 8.47-8.32 (m, 2H), 8.14-7.97 (m, 2H), 8.01 (s, 1H), 7.71 (d, J=6.8 Hz, 1H), 7.32-7.15 (m, 4H), 6.98-6.87 (m, 1H), 5.49 (s, 2H), 4.86-4.80 (m, 2H), 4.12 (t, J=9.4 Hz, 2H), 4.01 (t, J=8.7 Hz, 2H), 3.59-3.50 (m, 4H), 3.35-3.29 (m, 3H), 3.26 (s, 3H); MS (ES+): 552.2 (M+1); (ES−): 550.2 (M−1).

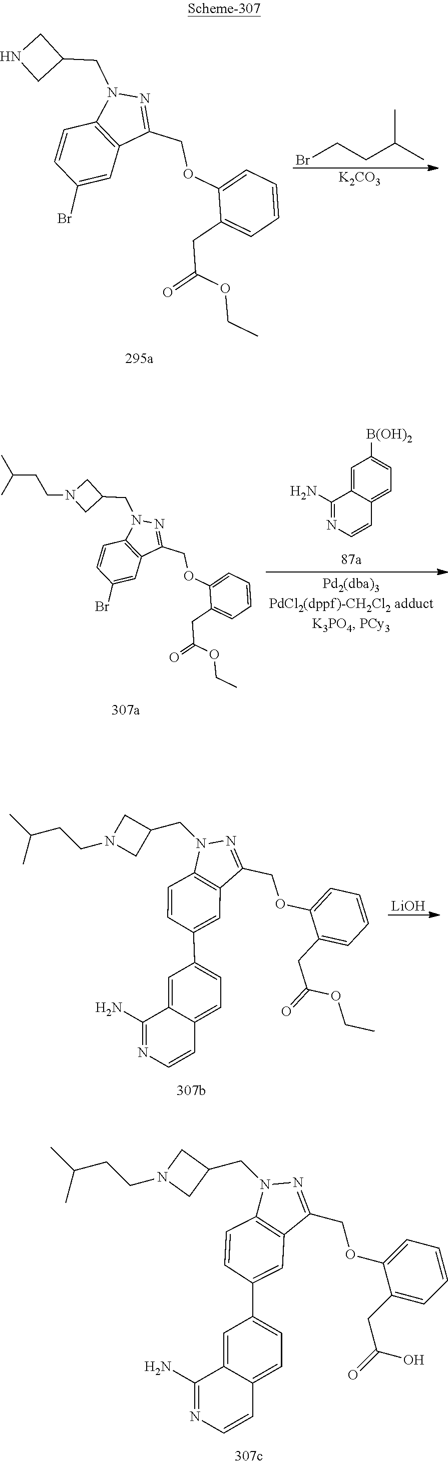

### Preparation of 2-(2-((5-(1-aminoisoquinolin-7-yl)-1-((1-isopentylazetidin-3-yl)methyl)-1H-indazol-3-yl)methoxy)phenyl)acetic acid (307c)

### Step-1: Preparation of ethyl 2-(2-((5-bromo-1-((1-isopentylazetidin-3-yl)methyl)-1H-indazol-3-yl)methoxy)phenyl)acetate (307a)

Compound 307a was prepared according to the procedure reported in step-2 of scheme-297, from ethyl 2-(2-((1-(azetidin-3-ylmethyl)-5-bromo-1H-indazol-3-yl)methoxy)phenyl)acetate (295a) (1 g, 2.182 mmol) in MeCN (15 mL) using 1-bromo-3-methylbutane (0.395 g, 2.62 mmol) and K₂CO₃ (0.754 g, 5.45 mmol) to afford after work up and purification using method-P, ethyl 2-(2-((5-bromo-1-((1-isopentylazetidin-3-yl)methyl)-1H-indazol-3-yl)methoxy)phenyl)acetate (307a) (796 mg, 69% yield); MS (ES+): 528.2 (M+1).

### Step-2: Preparation of ethyl 2-(2-((5-(1-aminoisoquinolin-7-yl)-1-((1-isopentylazetidin-3-yl)methyl)-1H-indazol-3-yl)methoxy)phenyl)acetate (307b)

Compound 307b was prepared according to the procedure reported in step-2 of scheme-18, from ethyl 2-(2-((5-bromo-1-((1-isopentylazetidin-3-yl)methyl)-1H-indazol-3-yl)methoxy)phenyl)acetate (307a) (275 mg, 0.520 mmol) in dioxane/MeTHF (5 mL, Ratio: 1:1) using (1-aminoisoquinolin-7-yl)boronic acid (87a) (127 mg, 0.676 mmol), aqueous tripotassium phosphate (4 M, 520 μL, 2.081 mmol), tricyclohexylphosphine (29.2 mg, 0.104 mmol), Pd₂(dba)₃ (47.7 mg, 0.052 mmol) and PdCl₂(dppf)-CH₂Cl₂Adduct (42.5 mg, 0.052 mmol) to afford after workup and purification using method-AE, ethyl 2-(2-((5-(1-aminoisoquinolin-7-yl)-1-((1-isopentylazetidin-3-yl)methyl)-1H-indazol-3-yl)methoxy)phenyl)acetate (307b) (158 mg, 51% yield) as a yellow solid; MS (ES+): 592.30 (M+1).

### Step-3: Preparation of 2-(2-((5-(1-aminoisoquinolin-7-yl)-1-((1-isopentylazetidin-3-yl)methyl)-1H-indazol-3-yl)methoxy)phenyl)acetic acid (307c)

Compound 307c was prepared according to the procedure reported in step-2 of scheme-1, from ethyl 2-(2-((5-(1-aminoisoquinolin-7-yl)-1-((1-isopentylazetidin-3-yl)methyl)-1H-indazol-3-yl)methoxy)phenyl)acetate (307b) (158 mg, 0.267 mmol) in THF (3 mL) using lithium hydroxide hydrate (33.6 mg, 0.801 mmol) in water (1 mL) to afford after work up and purification using method-M, 2-(2-((5-(1-aminoisoquinolin-7-yl)-1-((1-isopentylazetidin-3-yl)methyl)-1H-indazol-3-yl)methoxy)phenyl)acetic acid (307c) (108.5 mg, 72% yield) HCl salt as a white solid; ¹H NMR (300 MHz, DMSO-d₆) δ 13.34 (s, 1H, D₂O Exchangeable), 12.12 (s, 1H, D₂O Exchangeable), 11.44 (s, 1H, D₂O Exchangeable), 9.19 (s, 2H, D₂O Exchangeable), 9.00 (s, 1H), 8.42 (d, J=8.5, 1.6 Hz, 1H), 8.37 (s, 1H), 8.28-7.86 (m, 3H), 7.71 (d, J=6.9 Hz, 1H), 7.34-7.21 (m, 3H), 7.21 (d, J=7.3 Hz, 1H), 7.01-6.88 (m, 1H), 5.50 (s, 2H), 4.99-4.93 (m, 1H), 4.78-4.72 (m, 1H), 4.11-4.05 (m, 2H), 3.96 (d, J=9.7 Hz, 2H), 3.54 (s, 2H), 3.15-3.09 (m, 3H), 1.63-1.57 (m, 1H), 1.39-1.33 (m, 2H), 0.88 (d, J=6.4 Hz, 6H); MS (ES+): 564.3 (M+1); (ES−): 562.2 (M−1).

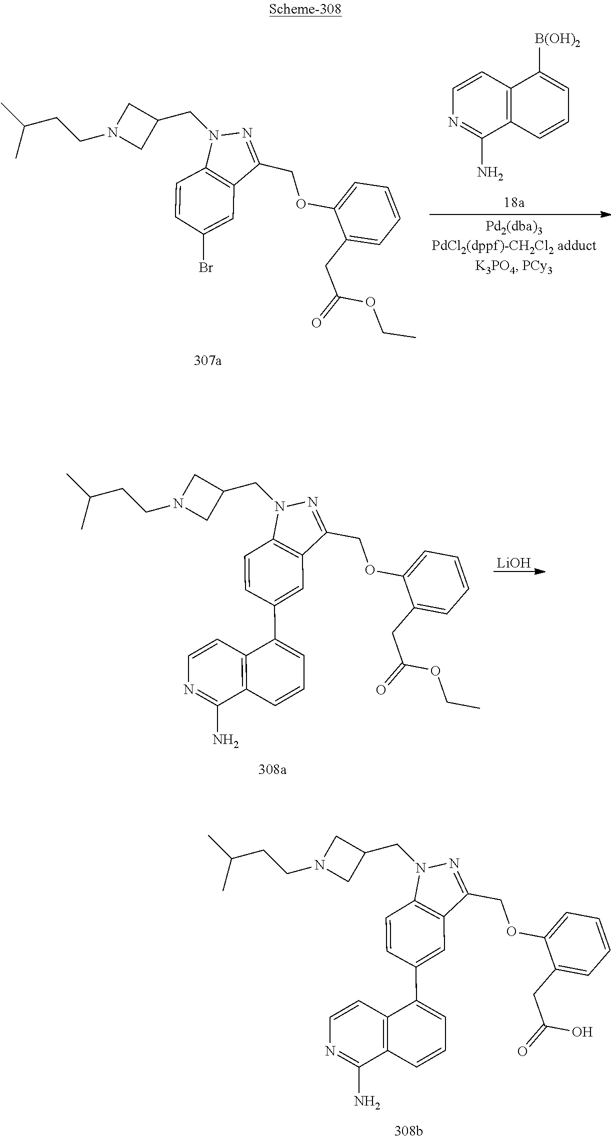

### Preparation of 2-(2-((5-(1-aminoisoquinolin-5-yl)-1-((1-isopentylazetidin-3-yl)methyl)-1H-indazol-3-yl)methoxy)phenyl)acetic acid (308b)

### Step-1: Preparation of ethyl 2-(2-((5-(1-aminoisoquinolin-5-yl)-1-((1-isopentylazetidin-3-yl)methyl)-1H-indazol-3-yl)methoxy)phenyl)acetate (308a)

Compound 308a was prepared according to the procedure reported in step-2 of scheme-18, from ethyl 2-(2-((5-bromo-1-((1-isopentylazetidin-3-yl)methyl)-1H-indazol-3-yl)methoxy)phenyl)acetate (307a) (275 mg, 0.520 mmol) in dioxane/MeTHF (S mL, Ratio: 1:1) using (I-aminoisoquinolin-5-yl)boronic acid (18a) (98 mg, 0.520 mmol), aqueous tripotassium phosphate (4 M, 520 μL, 2.081 mmol), tricyclohexylphosphine (29.2 mg, 0.104 mmol), Pd₂(dba)₃ (47.7 mg, 0.052 mmol) and PdCl₂(dppf)-CH₂Cl₂Adduct (42.5 mg, 0.052 mmol) to afford after workup and purification using method-AE, ethyl 2-(2-((5-(1-aminoisoquinolin-5-yl)-1-((1-isopentylazetidin-3-yl)methyl)-1H-indazol-3-yl)methoxy)phenyl)acetate (308a) (168 mg, 55% yield) as a yellow solid; MS (ES+): 592.4 (M+1).

### Step-2: Preparation of 2-(2-((5-(1-aminoisoquinolin-5-yl)-1-((1-isopentylazetidin-3-yl)methyl)-1H-indazol-3-yl)methoxy)phenyl)acetic acid (308b)

Compound 308b was prepared according to the procedure reported in step-2 of scheme-1, from ethyl 2-(2-((5-(1-aminoisoquinolin-5-yl)-1-((1-isopentylazetidin-3-yl)methyl)-1H-indazol-3-yl)methoxy)phenyl)acetate (308a) (165 mg, 0.279 mmol) in THF (3 mL) using lithium hydroxide hydrate (35.1 mg, 0.836 mmol) in water (1 mL) to afford after work up and purification using method-M, 2-(2-((5-(1-aminoisoquinolin-5-yl)-1-((1-isopentylazetidin-3-yl)methyl)-1H-indazol-3-yl)methoxy)phenyl)acetic acid (308b) (19.2 mg, 12% yield) HCl salt as a white solid; ¹H NMR (300 MHz, DMSO-d₆) δ 13.29 (s, 1H, D₂O exchangeable), 12.01 (s, 1H, DO exchangeable), 9.10 (s, 2H, D₂O exchangeable), 8.62 (d, J=8.3 Hz, 1H), 8.08 (d, J=8.8 Hz, 1H), 7.99-7.80 (m, 3H), 7.64 (d, J=7.2 Hz, 1H), 7.54 (dd, J=8.7, 1.6 Hz, 1H), 7.25 (d, J=4.0 Hz, 2H), 7.19 (d, J=7.4 Hz, 1H), 7.01-6.86 (m, 2H), 5.46 (s, 2H), 4.94 (d, J=7.9 Hz, 1H), 4.75 (d, J=6.9 Hz, 1H), 4.27-3.84 (m, 4H), 3.48 (s, 2H), 3.23-3.03 (m, 3H), 1.63-1.57 (m, 1H), 1.38-1.32 (m, 2H), 0.88 (t, J=6.3 Hz, 6H); MS (ES+): 564.3 (M+1); (ES−): 562.2 (M−1).

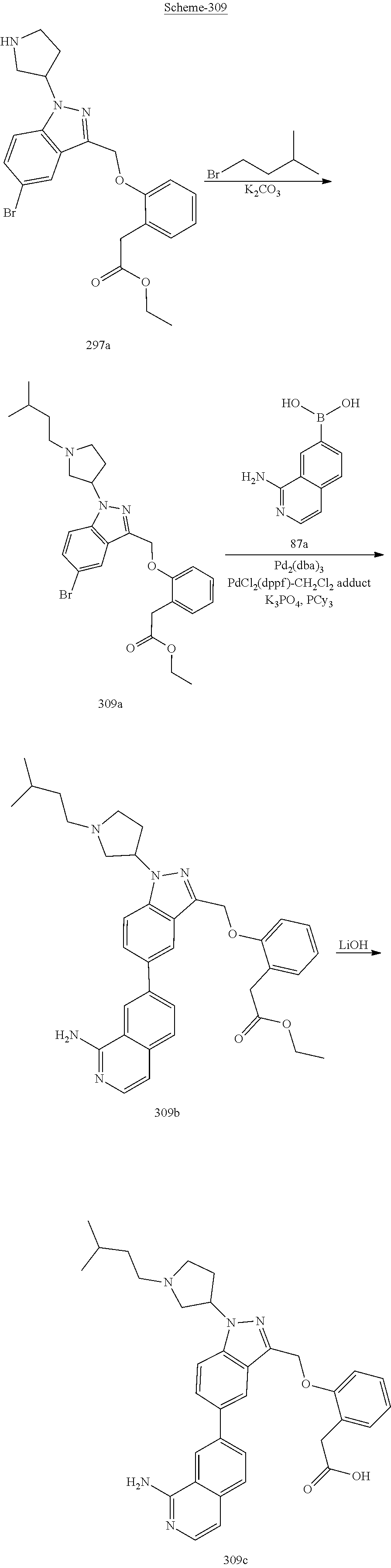

### Preparation of 2-(2-((5-(1-aminoisoquinolin-7-yl)-1-(1-isopentylpyrrolidin-3-yl)-1H-indazol-3-yl)methoxy)phenyl)acetic acid (309c)

### Step-1: Preparation of ethyl 2-(2-((5-bromo-1-(1-isopentylpyrrolidin-3-yl)-1H-indazol-3-yl)methoxy)phenyl)acetate (309a)

Compound 309a was prepared according to the procedure reported in step-2 of scheme-297, from ethyl 2-(2-((5-bromo-1-(pyrrolidin-3-yl)-1H-indazol-3-yl)methoxy)phenyl)acetate (297a) (1 g, 2.182 mmol) in MeCN (10 mL) using 1-bromo-3-methylbutane (0.395 g, 2.62 mmol) and K₂CO₃ (0.754 g, 5.45 mmol) to afford after work up and purification using method-P, ethyl 2-(2-((5-bromo-1-(1-isopentylpyrrolidin-3-yl)-1H-indazol-3-yl)methoxy)phenyl)acetate (309a) (670 mg, 58% yield); MS (ES+): 528.2 (M+1).

### Step-2: Preparation of ethyl 2-(2-((5-(1-aminoisoquinolin-7-yl)-1-(1-isopentylpyrrolidin-3-yl)-1H-indazol-3-yl)methoxy)phenyl)acetate (309b)

Compound 309b was prepared according to the procedure reported in step-2 of scheme-18, from ethyl 2-(2-((5-bromo-1-(1-isopentylpyrrolidin-3-yl)-1H-indazol-3-yl)methoxy)phenyl)acetate (309a) (220 mg, 0.416 mmol) in dioxane/MeTHF (5 mL, Ratio: 1:1) using (1-aminoisoquinolin-7-yl)boronic acid (87a) (102 mg, 0.541 mmol), aqueous tripotassium phosphate (4 M, 416 μl, 1.665 mmol), tricyclohexylphosphine (23.35 mg, 0.083 mmol), Pd₂(dba)₃ (38.1 mg, 0.042 mmol) and PdCl₂(dppf)-CH₂Cl₂Adduct (34.0 mg, 0.042 mmol) to afford after workup and purification using method-AE, ethyl 2-(2-((5-(1-aminoisoquinolin-7-yl)-1-(1-isopentylpyrrolidin-3-yl)-1H-indazol-3-yl)methoxy)phenyl)acetate (309b) (98 mg, 0.166 mmol, 39.8% yield) as a yellow solid; MS (ES+): 592.3 (M+1).

### Step-3: Preparation of 2-(2-((5-(1-aminoisoquinolin-7-yl)-1-(1-isopentylpyrrolidin-3-yl)-1H-indazol-3-yl)methoxy)phenyl)acetic acid (309c)

Compound 309c was prepared according to the procedure reported in step-2 of scheme-1, from ethyl 2-(2-((5-(1-aminoisoquinolin-7-yl)-1-(1-isopentylpyrrolidin-3-yl)-1H-indazol-3-yl)methoxy)phenyl)acetate (309b) (98 mg, 0.166 mmol) in THF (3 mL) using lithium hydroxide hydrate (20.85 mg, 0.497 mmol) in water (1 mL) to afford after work up and purification using method-M, 2-(2-((5-(1-aminoisoquinolin-7-yl)-1-(1-isopentylpyrrolidin-3-yl)-1H-indazol-3-yl)methoxy)phenyl)acetic acid (309c) (46.8 mg, 50% yield) HCl salt as a white solid; ¹H NMR (300 MHz, DMSO-d₆) δ 9.12 (s, 2H, D₂O exchangeable), 9.02-8.88 (m, 1H), 8.48-8.32 (m, 2H), 8.13-8.02 (m, 2H), 7.96 (d, J=8.9 Hz, 1H), 7.71 (d, J=6.9 Hz, 1H), 7.33-7.27 (m, 2H), 7.27-7.19 (m, 2H), 7.04-6.85 (m, 1H), 5.88-5.68 (m, 1H), 5.54 (s, 2H), 4.29-3.65 (m, 4H), 3.55 (s, 2H), 3.41-3.35 (m, 2H), 2.76-2.55 (m, 1H), 2.47-2.27 (m, 1H), 1.78-1.56 (m, 3H), 0.93 (d, J=5.9 Hz, 6H); MS (ES+): 564.3 (M+1); (ES−): 562.2 (M−1).

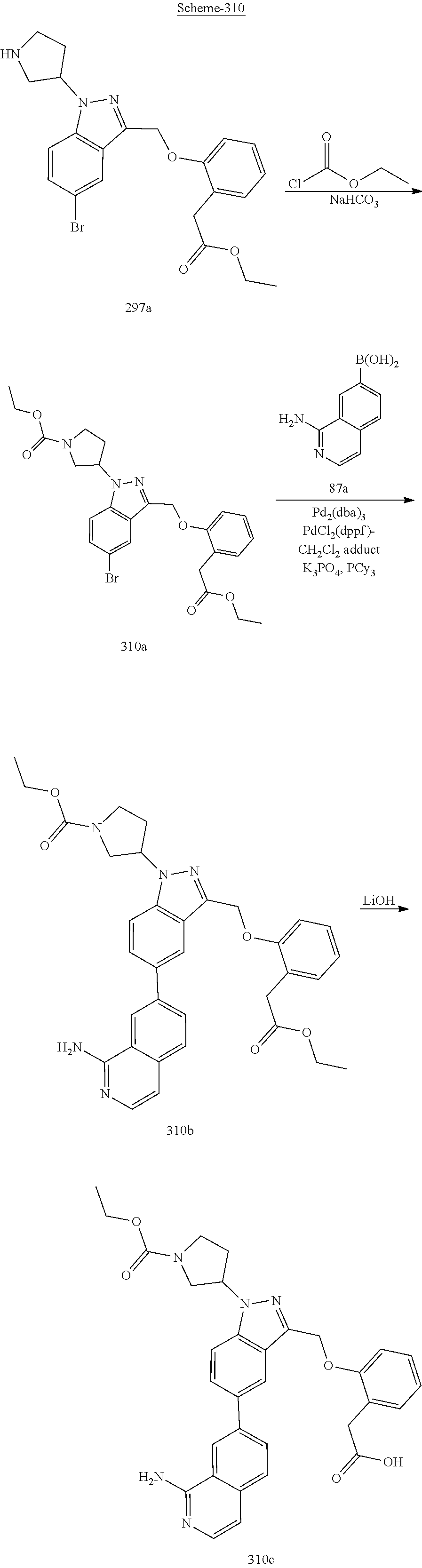

### Preparation of 2-(2-((5-(1-aminoisoquinolin-7-yl)-1-(1-(ethoxycarbonyl)pyrrolidin-3-yl)-1H-indazol-3-yl)methoxy)phenyl)acetic acid (310c)

### Step-1: Preparation of ethyl 3-(5-bromo-3-((2-(2-ethoxy-2-oxoethyl)phenoxy)methyl)-1H-indazol-1-yl)pyrrolidine-1-carboxylate (310a)

Compound 310a was prepared according to the procedure reported in step-1 of scheme-126, from ethyl 2-(2-((5-bromo-1-(pyrrolidin-3-yl)-1H-indazol-3-yl)methoxy)phenyl)acetate (297a) (1.75 g, 3.82 mmol) in THF (5 mL) and water (5 mL) using sodium bicarbonate (0.481 g, 5.73 mmol) and ethyl carbonochloridate (0.621 g, 5.73 mmol) to afford after workup and purification using method-J, ethyl 3-(5-bromo-3-((2-(2-ethoxy-2-oxoethyl)phenoxy)methyl)-1H-indazol-1-yl)pyrrolidine-1-carboxylate (310a) (743 mg, 37% yield) as a clear oil; (ES+): 530.2 (M+1)

### Step-2: Preparation of ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (310b)

Compound 310b was prepared according to the procedure reported in step-2 of scheme-18, from ethyl 3-(5-bromo-3-((2-(2-ethoxy-2-oxoethyl)phenoxy)methyl)-1H-indazol-1-yl)pyrrolidine-1-carboxylate (310a) (248 mg, 0.468 mmol) in dioxane/MeTHF (5 mL, Ratio: 1:1) using (1-aminoisoquinolin-7-yl)boronic acid (87a) (114 mg, 0.608 mmol), aqueous tripotassium phosphate (4 M, 468 μl, 1.870 mmol), tricyclohexylphosphine (26.2 mg, 0.094 mmol), Pd₂(dba)₃ (42.8 mg, 0.047 mmol) and PdCl₂(dppf)-CH₂Cl₂Adduct (38.2 mg, 0.047 mmol) to afford after workup and purification using method-AE, ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (310b) (156 mg, 56.2% yield) as a yellow solid; MS (ES+): 594.3 (M+1).

### Step-3: Preparation of 2-(2-((5-(1-aminoisoquinolin-7-yl)-1-(1-(ethoxycarbonyl)pyrrolidin-3-yl)-1H-indazol-3-yl)methoxy)phenyl)acetic acid (310c)

Compound 310c was prepared according to the procedure reported in step-2 of scheme-1, from ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (310b) (156 mg, 0.263 mmol) in THF (3 mL) using lithium hydroxide hydrate (33.1 mg, 0.788 mmol) in water (1 mL) to afford after work up and purification using method-M, 2-(2-((5-(1-aminoisoquinolin-7-yl)-1-(1-(ethoxycarbonyl)pyrrolidin-3-yl)-1H-indazol-3-yl)methoxy)phenyl)acetic acid (310c) (52.0 mg, 35% yield) HCl salt as a white solid; ¹H NMR (300 MHz, DMSO-d₆) δ 13.14 (s, 1H, D₂O Exchangeable), 12.11 (s, 1H, D₂O Exchangeable) 9.04 (s, 2H, D₂O Exchangeable), 8.94 (s, 1H), 8.41 (dd, J=8.5, 1.6 Hz, 1H), 8.32 (d, J=1.6 Hz, 1H), 8.08-7.99 (m, 2H), 7.99-7.90 (m, 1H), 7.69 (d, J=6.9 Hz, 1H), 7.33-7.19 (m, 3H), 7.19 (d, 1H), 6.97-6.86 (m, 1H), 5.58-5.52 (m, 1H), 5.48 (s, 2H), 4.07 (t, J=7.0 Hz, 2H), 3.91-3.85 (m, 1H), 3.75-3.59 (m, 2H), 3.59-3.51 (m, 1H), 3.53 (s, 2H), 2.49-2.39 (m, 2H), 1.28-1.13 (m, 3H); MS (ES+): 566.2 (M+1); (ES−): 564.2 (M−1).

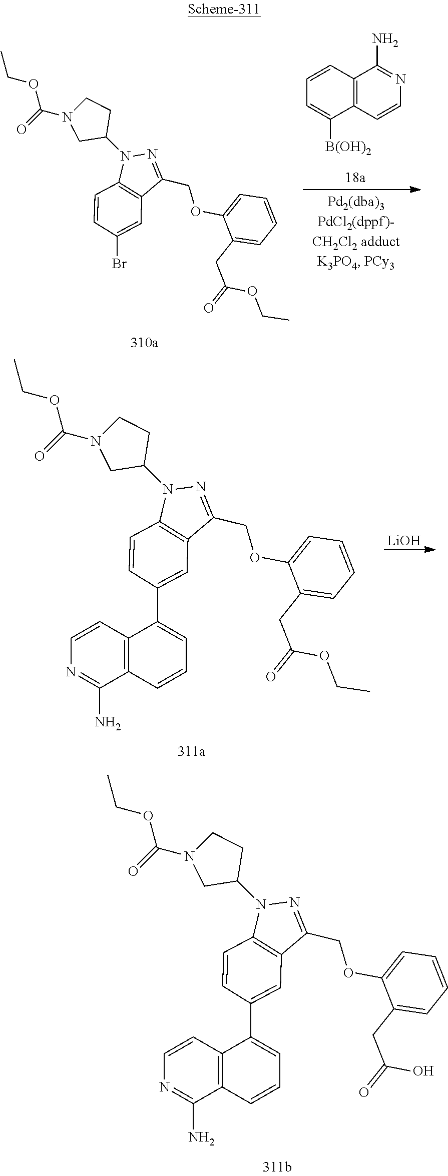

### Preparation of 2-(2-((5-(1-aminoisoquinolin-5-yl)-1-(1-(ethoxycarbonyl)pyrrolidin-3-yl)-1H-indazol-3-yl)methoxy)phenyl)acetic acid (311b)

### Step-1: Preparation of ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (311a)

Compound 311a was prepared according to the procedure reported in step-2 of scheme-18, from ethyl 3-(5-bromo-3-((2-(2-ethoxy-2-oxoethyl)phenoxy)methyl)-1H-indazol-1-yl)pyrrolidine-1-carboxylate (310a) (248 mg, 0.468 mmol) in dioxane/MeTHF (5 mL, Ratio: 1:1) using (1-aminoisoquinolin-5-yl)boronic acid (18a) (88 mg, 0.468 mmol), aqueous tripotassium phosphate (4 M, 468 μl, 1.870 mmol), tricyclohexylphosphine (26.2 mg, 0.094 mmol), Pd₂(dba)₃ (42.8 mg, 0.047 mmol) and PdCl₂(dppf)-CH₂Cl₂Adduct (38.2 mg, 0.047 mmol) to afford after workup and purification using method-AE, ethyl 3-(5-(1-aminoisoquinolin-5-yl)˜3-((2-(2-ethoxy-2-oxoethyl)phenoxy)methyl)-1H-indazol-1-yl)pyrrolidine-1-carboxylate (311a) (172 mg, 62% yield) as a yellow solid; MS (ES+): 594.3 (M+1).

### Step-2: Preparation of 2-(2-((5-(1-aminoisoquinolin-5-yl)-1-(1-(ethoxycarbonyl)pyrrolidin-3-yl)-1H-indazol-3-yl)methoxy)phenyl)acetic acid (311b)

Compound 311b was prepared according to the procedure reported in step-2 of scheme-1, from ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (311a) (174.0 mg, 293 mmol) in THF (3 mL) using lithium hydroxide hydrate (36.9 mg, 879 mmol) in water (1 mL) to afford after work up and purification using method-M, 2-(2-((5-(1-aminoisoquinolin-5-yl)-1-(1-(ethoxycarbonyl)pyrrolidin-3-yl)-1H-indazol-3-yl)methoxy)phenyl)acetic acid (311b) (151.3 mg, 91% yield) HCl salt as a white solid; ¹H NMR (300 MHz, DMSO-d₆) δ 13.36 (s, 1H, D₂O Exchangeable), 11.99 (s, 1H, D₂O Exchangeable), 9.16 (s, 2H, D₂O Exchangeable), 8.63 (d, J=8.3 Hz, 1H), 8.00-7.87 (m, 3H), 7.91-7.79 (m, 1H), 7.63 (d, J=7.2 Hz, 1H), 7.51 (dd, J=8.6, 1.7 Hz, 1H), 7.30-7.16 (m, 2H), 7.21-7.12 (m, 1H), 6.97 (d, J=7.2 Hz, 1H), 6.94-6.84 (m, 1H), 5.59-5.52 (m, 1H), 5.45 (s, 2H), 4.12-4.02 (m, 2H), 3.93-3.85 (m, 1H), 3.71 (dd, J=11.1, 4.7 Hz, 1H), 3.67-3.62 (m, 1H), 3.57-3.51 (m, 1H), 3.47 (s, 2H), 2.45-2.39 (m, 2H), 1.28-1.13 (m, 3H); MS (ES+): 566.2 (M+1); (ES−): 564.2 (M−1).

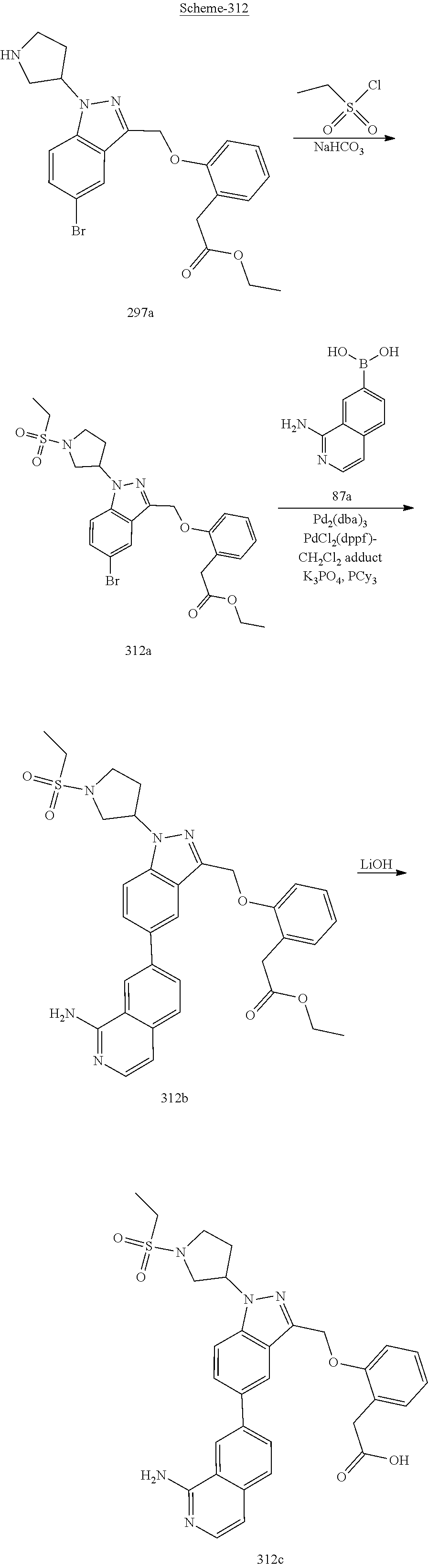

### Preparation of 2-(2-((5-(1-aminoisoquinolin-7-yl)-1-(1-(ethylsulfonyl)pyrrolidin-3-yl)-1H-indazol-3-yl)methoxy)phenyl)acetic acid (312c)

### Step-1: Preparation of ethyl 2-(2-((5-bromo-1-(1-(ethylsulfonyl)pyrrolidin-3-yl)-1H-indazol-3-yl)methoxy)phenyl)acetate (312a)

To a solution of ethyl 2-(2-((5-bromo-1-(pyrrolidin-3-yl)-1H-indazol-3-yl)methoxy)phenyl)acetate (297a) (1 g, 2.182 mmol) in THF (10 mL) and water (10 mL) was added sodium bicarbonate (0.275 g, 3.27 mmol) and ethane sulfonyl chloride (0.281 g, 2.182 mmol) at 0° C. and the mixture was stirred overnight at RT. The reaction mixture was diluted with EtOAc (100 mL), washed with water, brine, dried, filtered and concentrated in vacuo. The residue obtained was purified using method-J, ethyl 2-(2-((5-bromo-1-(1-(ethylsulfonyl)pyrrolidin-3-yl)-1H-indazol-3-yl)methoxy)phenyl)acetate (312a) (956 mg, 80% yield) as a clear oil; MS (ES+): 550.1 (M+1).

### Step-2: Preparation of ethyl 2-(2-((5-(1-aminoisoquinolin-7-yl)-1-(1-(ethylsulfonyl)pyrrolidin-3-yl)-1H-indazol-3-yl)methoxy)phenyl)acetate (312b)

Compound 312b was prepared according to the procedure reported in step-2 of scheme-18, from ethyl 2-(2-((5-bromo-1-(1-(ethylsulfonyl)pyrrolidin-3-yl)-1H-indazol-3-yl)methoxy)phenyl)acetate (312a) (265 mg, 0.481 mmol) in dioxane/MeTHF (5 mL, Ratio: 1:1) using (1-aminoisoquinolin-7-yl)boronic acid (87a) (118 mg, 0.626 mmol), aqueous tripotassium phosphate (4 M, 481 μl, 1.926 mmol), tricyclohexylphosphine (27.0 mg, 0.096 mmol), Pd₂(dba)₃ (44.1 mg, 0.048 mmol) and PdCl₂(dppf)-CH₂Cl₂Adduct (39.3 mg, 0.048 mmol) to afford after workup and purification using method-AE, ethyl 2-(2-((5-(1-aminoisoquinolin-7-yl)-1-(1-(ethylsulfonyl)pyrrolidin-3-yl)-1H-indazol-3-yl)methoxy)phenyl)acetate (312b) (198 mg, 67% yield) as a yellow solid; MS (ES+): 614.2 (M+1).

### Step-3: Preparation of 2-(2-((5-(1-aminoisoquinolin-7-yl)-1-(1-(ethylsulfonyl)pyrrolidin-3-yl)-1H-indazol-3-yl)methoxy)phenyl)acetic acid (312c)

Compound 312c was prepared according to the procedure reported in step-2 of scheme-1, from ethyl 2-(2-((5-(1-aminoisoquinolin-7-yl)-1-(1-(ethylsulfonyl)pyrrolidin-3-yl)-1H-indazol-3-yl)methoxy)phenyl)acetate (312b) (150 mg, 0.244 mmol) in THF (3 mL) using lithium hydroxide hydrate (30.8 mg, 0.733 mmol) in water (1 mL) to afford after work up and purification using method-M, 2-(2-((5-(1-aminoisoquinolin-7-yl)-1-(1-(ethylsulfonyl)pyrrolidin-3-yl)-1H-indazol-3-yl)methoxy)phenyl)acetic acid (312c) (38.1 mg, 27% yield) HCl salt as a white solid; ¹H NMR (300 MHz, DMSO-d₆) δ 13.13 (s, 1H, D₂O Exchangeable), 12.09 (s, 1H, D₂O Exchangeable) 9.04 (s, 2H, D₂O Exchangeable), 8.95 (s, 1H), 8.42 (dd, J=8.5, 1.6 Hz, 1H), 8.38-8.32 (m, 1H), 8.10-8.01 (m, 2H), 7.97 (d, J=8.9 Hz, 1H), 7.70 (d, J=6.9 Hz, 1H), 7.34-7.24 (m, 3H), 7.24-7.17 (m, 1H), 6.99-6.88 (m, 1H), 5.68-5.56 (m, 1H), 5.51 (s, 2H), 3.98-3.86 (m, 1H), 3.74-3.60 (m, 2H), 3.60-3.48 (m, 3H), 3.27-3.07 (m, 2H), 2.60-2.37 (m, 2H), 1.25 (t, J=7.4 Hz, 3H); MS (ES+): 586.2 (M+1); (ES−): 584.2 (M−1).

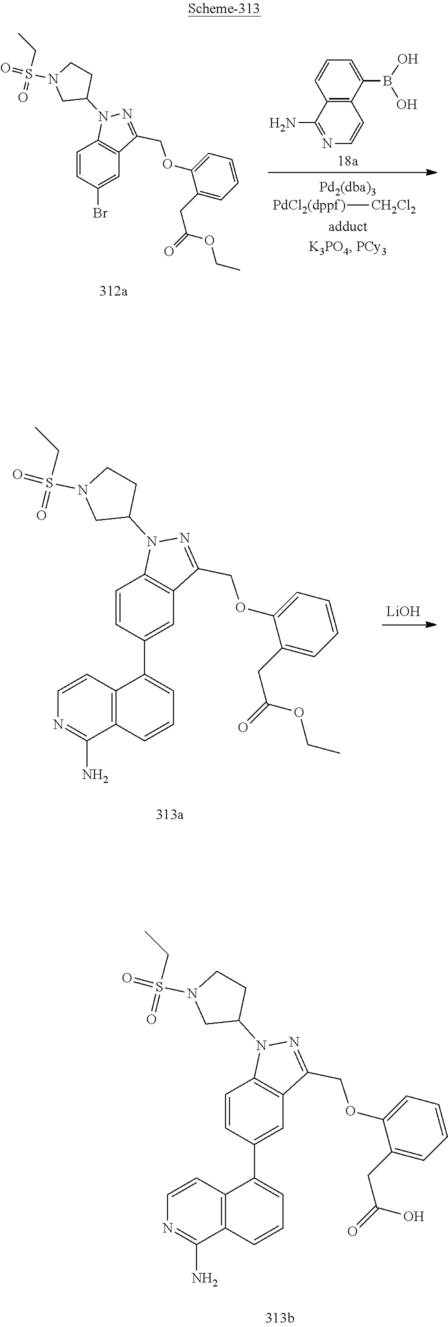

### Preparation of 2-(2-((5-(1-aminoisoquinolin-5-yl)-1-(1-(ethylsulfonyl)pyrrolidin-3-yl)-1H-indazol-3-yl)methoxy)phenyl)acetic acid (313b)

### Step-1: Preparation of ethyl 2-(2-((5-(1-aminoisoquinolin-5-yl)˜1-(1-(ethylsulfonyl)pyrrolidin-3-yl)-1H-indazol-3-yl)methoxy)phenyl)acetate (313a)

Compound 313a was prepared according to the procedure reported in step-2 of scheme-18, from ethyl 2-(2-((5-bromo-1-(1-(ethylsulfonyl)pyrrolidin-3-yl)-1H-indazol-3-yl)methoxy)phenyl)acetate (312a) (265 mg, 0.481 mmol) in dioxane/MeTHF (5 mL, Ratio: 1:1) using (1-aminoisoquinolin-5-yl)boronic acid (18a) (91 mg, 0.481 mmol), aqueous tripotassium phosphate (4 M, 481 μl, 1.926 mmol), tricyclohexylphosphine (27.0 mg, 0.096 mmol) Pd₂(dba)₃ (44.1 mg, 0.048 mmol) and PdCl₂(dppf)-CH₂Cl₂Adduct (39.3 mg, 0.048 mmol) to afford after workup and purification using method-AE, ethyl 2-(2-((5-(1-aminoisoquinolin-5-yl)-1-(1-(ethylsulfonyl)pyrrolidin-3-yl)-1H-indazol-3-yl)methoxy)phenyl)acetate (313a) (181 mg, 61% yield) as a yellow solid; MS (ES+): 614.2 (M+1).

### Step-2: Preparation of 2-(2-((5-(1-aminoisoquinolin-5-yl)-1-(1-(ethylsulfonyl)pyrrolidin-3-yl)-1H-indazol-3-yl)methoxy)phenyl)acetic acid (313b)

Compound 313b was prepared according to the procedure reported in step-2 of scheme-1, from ethyl 2-(2-((5-(1-aminoisoquinolin-5-yl)-1-(1-(ethylsulfonyl)pyrrolidin-3-yl)-1H-indazol-3-yl)methoxy)phenyl)acetate (313a) (181 mg, 0.295 mmol) in THF (3 mL) using lithium hydroxide hydrate (37.1 mg, 0.885 mmol) in water (1 mL) to afford after work up and purification using method-M, 2-(2-((5-(1-aminoisoquinolin-5-yl)-1-(1-(ethylsulfonyl)pyrrolidin-3-yl)-1H-indazol-3-yl)methoxy)phenyl)acetic acid (313b) (40.0 mg, 23% yield) HCl salt as a white solid; ¹H NMR (300 MHz, DMSO-d₆) δ 13.17 (s, 1H, D₂O Exchangeable), 12.00 (s, 1H, D₂O Exchangeable), 9.08 (s, 2H, D₂O Exchangeable), 8.61 (d, J=8.3 Hz, 1H), 8.00-7.90 (m, 3H), 7.90-7.80 (m, 1H), 7.62 (d, J=7.3 Hz, 1H), 7.52 (dd, J=8.7, 1.7 Hz, 1H), 7.31-7.18 (m, 2H), 7.22-7.13 (m, 1H), 6.98 (d, J=7.2 Hz, 1H), 6.95-6.85 (m, 1H), 5.69-5.57 (m, 1H), 5.47 (s, 2H), 3.99-3.87 (m, 1H), 3.75-3.61 (m, 2H), 3.61-3.50 (m, 1H), 3.48 (s, 2H), 3.29-3.09 (m, 2H), 2.50-2.38 (m, 2H), 1.26 (t, J=7.4 Hz, 3H); MS (ES+): 586.2 (M+1); (ES−): 584.2 (M−1).

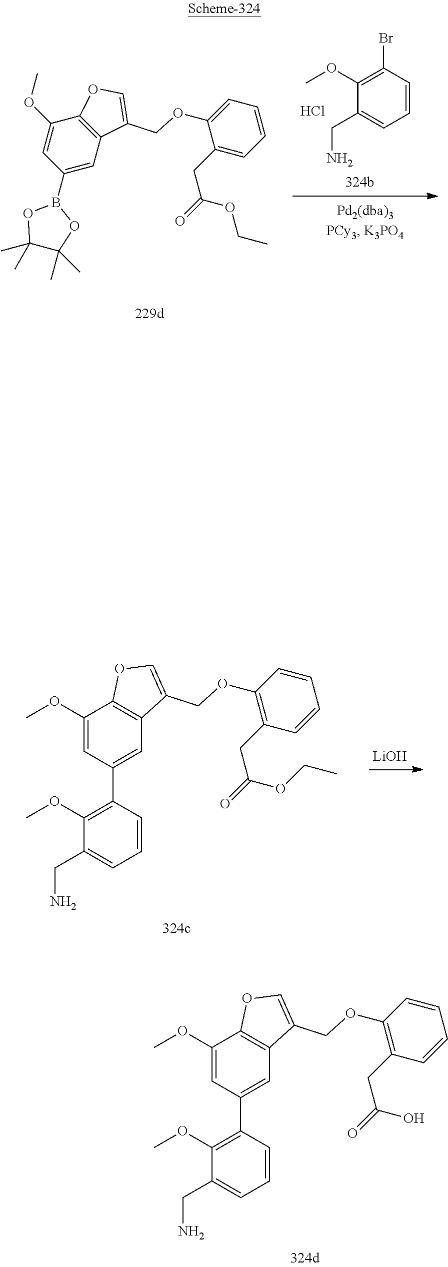

### Preparation of 2-(2-((5-(3-(aminomethyl)-2-methoxyphenyl)-7-methoxybenzofuran-3-yl)methoxy)phenyl)acetic acid (324d)

### Step-1: Preparation of ethyl 2-(2-((5-(3-(aminomethyl)-2-methoxyphenyl)-7-methoxybenzofuran-3-yl)methoxy)phenyl)acetate (324c)

Compound 324c was prepared according to the procedure reported in step-3 of scheme 112, from ethyl 2-(2-((7-methoxy-5-(4,4,5,5-tetramethyl-1,3,2-dioxaborolan-2-yl)benzofuran-3-yl)methoxy)phenyl)acetate (229d) (0.24 g, 0.515 mmol) in dioxane (18 mL) using (3-bromo-2-methoxyphenyl)methanamine hydrochloride (324b) (0.195 g, 0.772 mmol), 4 M aqueous solution of K₃PO₄ (0.515 mL, 2.059 mmol), tricyclohexylphosphine (0.043 g, 0.154 mmol), Pd₂(dba)₃ (0.071 g, 0.077 mmol) and heating at 115° C. for 3 h to afford after workup and purification using method-AM, ethyl 2-(2-((5-(3-(aminomethyl)-2-methoxyphenyl)-7-methoxybenzofuran-3-yl)methoxy)phenyl)acetate (324c) (166 mg, 68% yield) as a clear oil; MS (ES+): 476.2 (M+1).

### Step-2: Preparation of 2-(2-((5-(3-(aminomethyl)-2-methoxyphenyl)-7-methoxybenzofuran-3-yl)methoxy)phenyl)acetic acid (324d)

Compound 324d was prepared according to the procedure reported in step-2 of scheme-1, from ethyl 2-(2-((5-(3-(aminomethyl)-2-methoxyphenyl)-7-methoxybenzofuran-3-yl)methoxy)phenyl)acetate (324c) (160 mg, 0.336 mmol) in THF (2 mL) and acetonitrile (1 mL) using a 1N solution of lithium hydroxide monohydrate (1.009 mL, 1.009 mmol) and stirring for 25 h at RT to afford after workup and purification using method-G, 2-(2-((5-(3-(aminomethyl)-2-methoxyphenyl)-7-methoxybenzofuran-3-yl)methoxy)phenyl)acetic acid (324d) (73 mg, 49% yield) HCl salt as an off-white solid; ¹H NMR (300 MHz, DMSO-d₆) & 12.11 (s, 1H, D₂O exchangeable), 8.36 (s, 3H, D₂O exchangeable), 8.11 (s, 1H), 7.55-7.47 (m, 2H), 7.45 (d, J=1.4 Hz, 1H), 7.33-7.13 (m, 5H), 6.92 (td, J=7.3, 1.2 Hz, 1H), 5.28 (s, 2H), 4.10 (s, 2H), 3.98 (s, 3H), 3.53 (s, 2H), 3.37 (s, 3H); MS (ES+): 448.1 (M+1); (ES−): 446.1 (M−1).

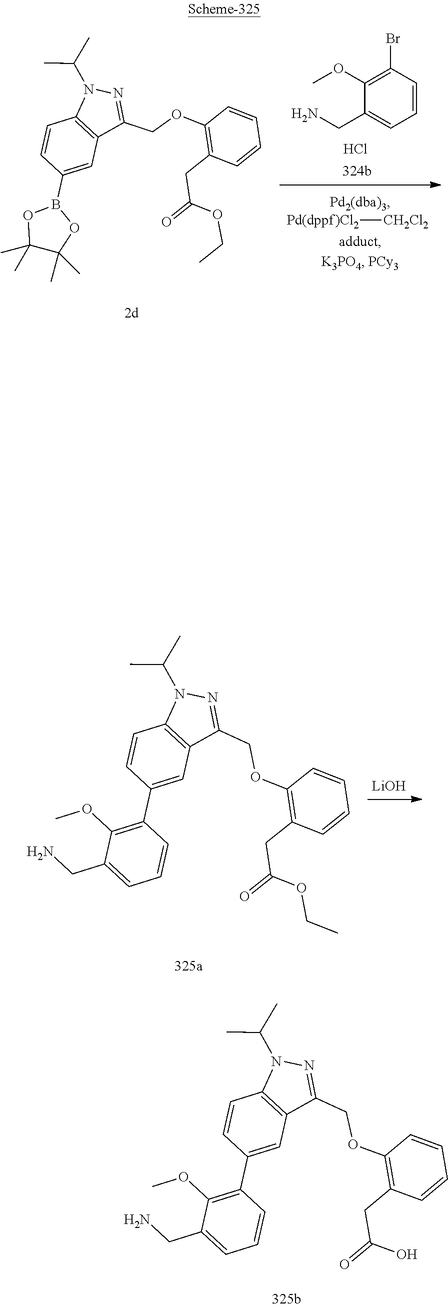

### Preparation of 2-(2-((5-(3-(aminomethyl)-2-methoxyphenyl)-1-isopropyl-1H-indazol-3-yl)methoxy)phenyl)acetic acid (325b)

### Step-1: Preparation of ethyl 2-(2-((5-(3-(aminomethyl)-2-methoxyphenyl)-1-isopropyl-1H-indazol-3-yl)methoxy)phenyl)acetate (325a)

Compound 325a was prepared according to the procedure reported in step-5 of scheme 1, from ethyl 2-(2-((1-isopropyl-5-(4,4,5,5-tetramethyl-1,3,2-dioxaborolan-2-yl)-1H-indazol-3-yl)methoxy) phenyl)acetate (2d) (200 mg, 0.418 mmol) in dioxane (2 mL) and THF (2 mL) using (3-bromo-2-methoxyphenyl)methanamine hydrochloride (324b) (211 mg, 0.836 mmol), 2 M solution of K₃PO₄ (0.836 mL, 1.672 mmol), tricyclohexylphosphine (23.45 mg, 0.084 mmol), Pd₂(dba)₃ (38.3 mg, 0.042 mmol), PdCl₂(dppf)-CH₂Cl₂ adduct (34.1 mg, 0.042 mmol) and heating at 100° C. for 2 h to afford after work up and purification using method-F, ethyl 2-(2-((5-(3-(aminomethyl)-2-methoxyphenyl)-1-isopropyl-1H-indazol-3-yl)methoxy)phenyl)acetate (325a) (150 mg, 74% yield) as a clear oil; ¹H NMR (300 MHZ, DMSO-d₆) δ 7.88 (s, 1H), 7.77 (d, J=8.9 Hz, 1H), 7.62 (t, J=7.8 Hz, 1H), 7.49-7.44 (m, 2H), 7.27 (d, J=3.9 Hz, 1H), 7.20-7.14 (m, 2H), 7.06 (t, J=7.6 Hz, 1H), 6.95-6.85 (m, 1H), 5.42 (s, 2H), 5.12-4.95 (m, 1H), 3.83-3.81 (m, 2H), 3.75 (s, 3H), 3.72-3.62 (m, 2H), 3.52 (s, 2H), 1.52 (d, J=6.5 Hz, 6H), 0.86-0.75 (m, 3H); MS (ES+): 488.30 (M+1).

### Step-2: Preparation of 2-(2-((5-(3-(aminomethyl)-2-methoxyphenyl)-1-isopropyl-1H-indazol-3-yl)methoxy)phenyl)acetic acid (325b)

Compound 325b was prepared according to the procedure reported in step-2 of scheme-1, from ethyl 2-(2-((5-(3-(aminomethyl)-2-methoxyphenyl)-1-isopropyl-1H-indazol-3-yl)methoxy)phenyl)acetate (325a) (150 mg, 0.308 mmol) in THF (2 mL) and MeOH (2 mL) using lithium hydroxide hydrate (77 mg, 1.846 mmol) in water (1 mL) and stirring at RT for 15 h to afford after work up and purification using method-M, 2-(2-((5-(3-(aminomethyl)-2-methoxyphenyl)-1-isopropyl-1H-indazol-3-yl)methoxy)phenyl)acetic acid (325b) (55 mg, 39% yield) HCl salt as a white solid; H NMR (300 MHz, DMSO-d₆) δ 12.07 (s, 1H, D₂O exchangeable), 8.33 (s, 3H, D₂O exchangeable), 7.95 (s, 1H), 7.81 (d, J=8.8 Hz, 1H), 7.61 (dd, J=8.8, 1.6 Hz, 1H), 7.47 (td, J=7.7, 1.7 Hz, 2H), 7.32-7.23 (m, 3H), 7.23-7.14 (m, 1H), 6.97-6.86 (m, 1H), 5.43 (s, 2H), 5.11-4.95 (m, 1H), 4.15-4.04 (m, 2H), 3.51 (s, 2H), 3.31 (s, 3H), 1.53 (d, J=6.6 Hz, 6H); MS (ES+): 460.20 (M+1); (ES−): 458.20 (M−1).

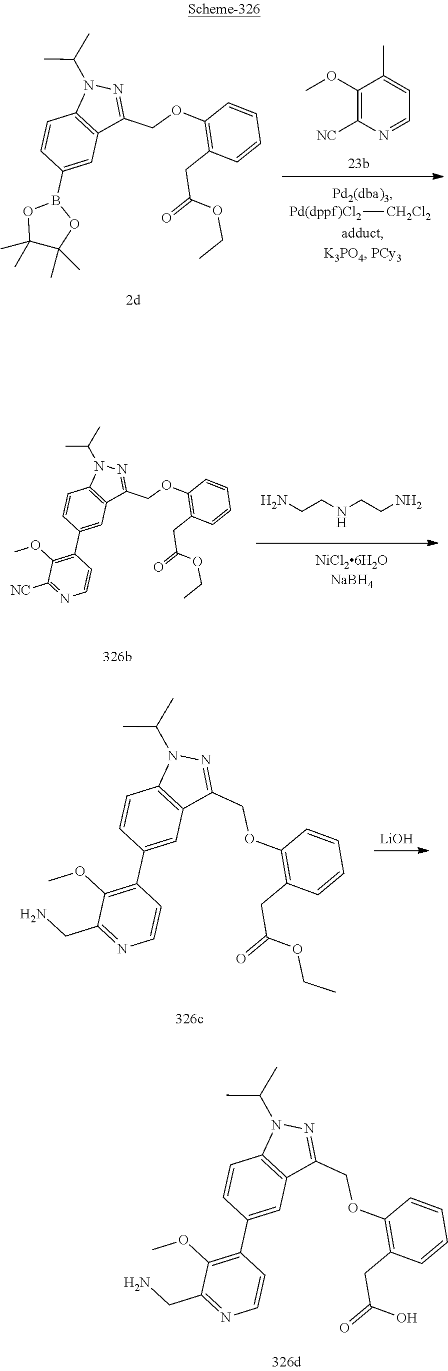

### Preparation of 2-(2-((5-(2-(aminomethyl)-3-methoxypyridin-4-yl)-1-isopropyl-1H-indazol-3-yl)methoxy)phenyl)acetic acid (326d)

### Step-1: Preparation of ethyl 2-(2-((5-(2-cyano-3-methoxypyridin-4-yl)-1-isopropyl-1H-indazol-3-yl)methoxy)phenyl)acetate (326b)

Compound 326b was prepared according to the procedure reported in step-5 of scheme 1, from ethyl 2-(2-(1-isopropyl-5-(4,4,5,5-tetramethyl-1,3,2-dioxaborolan-2-yl)-1H-indazol-3-yl)methoxy)phenyl)acetate (2d) (300 mg, 0.627 mmol) in dioxane (2 mL) and THF (2 mL) using 4-iodo-3-methoxypicolinonitrile (23b) (196 mg, 0.753 mmol), 2 M aqueous solution of K₃PO₄ (1.254 mL, 2.508 mmol), tricyclohexylphosphine (35.2 mg, 0.125 mmol), Pd₂(dba)₃ (57.4 mg, 0.063 mmol), PdCl₂(dppf)-CH₂Cl₂ adduct (51.2 mg, 0.063 mmol) and heating at 100° C. for 2 h to afford after workup and purification using method-AC, ethyl 2-(2-((5-(2-cyano-3-methoxypyridin-4-yl)-1-isopropyl-1H-indazol-3-yl)methoxy)phenyl)acetate (326b) (180 mg, 59% yield) as a clear oil; ¹H NMR (300 MHz, DMSO-d₆) δ 8.55 (d, J=4.9 Hz, 1H), 8.12-8.04 (m, 1H), 7.93-7.87 (m, 1H), 7.84 (d, J=4.9 Hz, 1H), 7.70 (dd, J=8.8, 1.6 Hz, 1H), 7.33-7.23 (m, 2H), 7.23-7.15 (m, 1H), 6.99-6.84 (m, 1H), 5.44 (s, 2H), 5.14-4.99 (m, 1H), 3.72-3.66 (m, 2H), 3.65 (s, 3H), 3.54 (s, 2H), 1.52 (d, J=6.6 Hz, 6H), 0.80 (t, J=7.1 Hz, 3H); MS (ES+): 485.20 (M+1); (ES−): 483.05 (M−1).

### Step-2: Preparation of ethyl 2-(2-((5-(2-(aminomethyl)-3-methoxypyridin-4-yl)-1-isopropyl-1H-indazol-3-yl)methoxy)phenyl)acetate (326c)

Compound 326c was prepared according to the procedure reported in scheme-287, from ethyl 2-(2-((5-(2-cyano-3-methoxypyridin-4-yl)-1-isopropyl-1H-indazol-3-yl)methoxy)phenyl)acetate (326b) (180 mg, 0.371 mmol) in anhydrous ethanol (10 mL) using nickel(II) chloride hexahydrate (22.07 mg, 0.093 mmol), sodium borohydride (42.2 mg, 1.114 mmol), N1-(2-aminoethyl)ethane-1,2-diamine (0.08 mL, 0.743 mmol) to afford after workup and purification using method-BI, to furnish ethyl 2-(2-((5-(2-(aminomethyl)-3-methoxypyridin-4-yl)-1-isopropyl-1H-indazol-3-yl)methoxy)phenyl)acetate (326c) (60 mg, 33% yield) as a white solid; ¹H NMR (300 MHz, DMSO-d₆) δ 8.35 (d, J=4.9 Hz, 1H), 8.02 (d, J=1.3 Hz, 1H), 7.84 (d, J=8.8 Hz, 1H), 7.74-7.65 (m, 1H), 7.37 (d, J=5.0 Hz, 1H), 7.28 (dd, J=4.8, 1.5 Hz, 2H), 7.18 (d, J=7.4 Hz, 1H), 6.95-6.85 (m, 1H), 5.43 (s, 2H), 5.05 (t, J=6.6 Hz, 1H), 3.90 (s, 2H), 3.67 (q, J=7.2 Hz, 2H), 3.56-3.50 (m, 2H), 3.37 (s, 3H), 1.52 (d, J=6.6 Hz, 6H), 0.78 (q, J=7.2 Hz, 3H); MS (ES+): 489.30 (M+1).

### Step-3: Preparation of 2-(2-((5-(2-(aminomethyl)-3-methoxypyridin-4-yl)-1-isopropyl-1H-indazol-3-yl)methoxy)phenyl)acetic acid (326d)

Compound 326d was prepared according to the procedure reported in step-2 of scheme 1, from ethyl 2-(2-((5-(2-(aminomethyl)-3-methoxypyridin-4-yl)-1-isopropyl-1H-indazol-3-yl)methoxy)phenyl)acetate (326c) (60 mg, 0.123 mmol) in THF (2 mL) and MeOH (2 mL) using lithium hydroxide hydrate (30.9 mg, 0.737 mmol) in water (I mL) and stirring at RT for 15 h. This gave after workup and purification using method-G, 2-(2-((5-(2-(aminomethyl)-3-methoxypyridin-4-yl)-1-isopropyl-1H-indazol-3-yl)methoxy)phenyl)acetic acid (326d) (25 mg, 44% yield) HCl salt as a light yellow solid; ¹H NMR (300 MHz, DMSO-d₆) δ 8.45 (d, J=5.0 Hz, 1H), 8.42-8.29 (m, 3H, D₂O exchangeable), 8.10 (d, J=1.5 Hz, 1H), 7.88 (d, J=8.9 Hz, 1H), 7.70 (dd, J=8.8, 1.6 Hz, 1H), 7.55 (d, J=5.0 Hz, 1H), 7.30-7.23 (m, 2H), 7.20 (d, J=7.3 Hz, 1H), 6.98-6.86 (m, 1H), 5.45 (s, 2H), 5.14-4.98 (m, 1H), 4.34-4.24 (m, 2H), 3.52 (s, 2H), 3.39 (s, 3H), 1.53 (d, J=6.6 Hz, 6H); MS (ES+): 461.20 (M+1); (ES−): 459.20 (M−1).

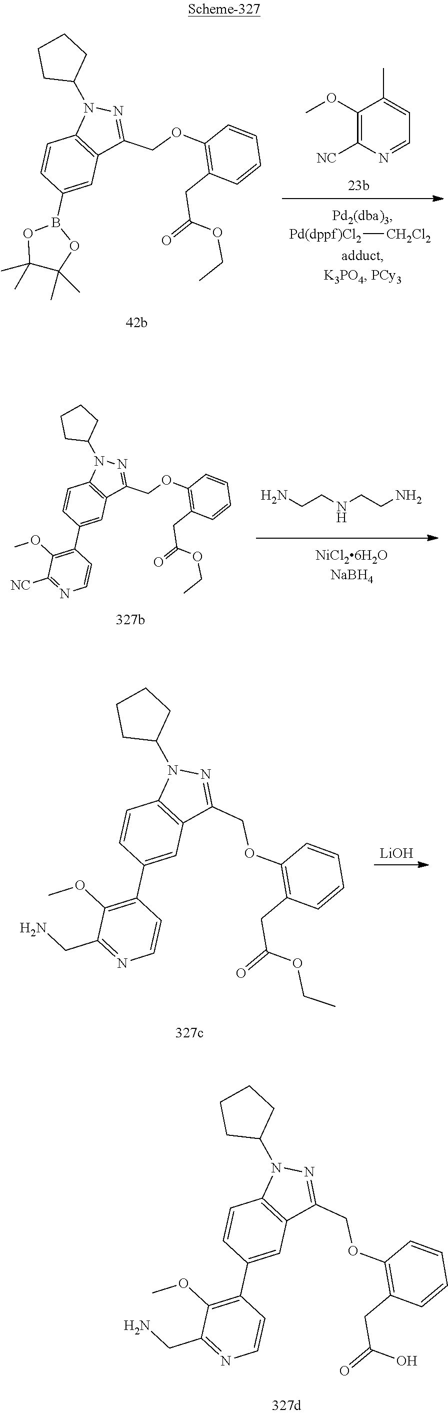

### Preparation of 2-(2-((5-(2-(aminomethyl)-3-methoxypyridin-4-yl)-1-cyclopentyl-1H-indazol-3-yl)methoxy)phenyl)acetic acid (327d)

### Step-1: Preparation of ethyl 2-(2-((5-(2-cyano-3-methoxypyridin-4-yl)-1-cyclopentyl-1H-indazol-3-yl)methoxy)phenyl)acetate (327b)

Compound 327b was prepared according to the procedure reported in step-5 of scheme 1, from ethyl 2-(2-((1-cyclopentyl-5-(4,4,5,5-tetramethyl-1,3,2-dioxaborolan-2-yl)-1H-indazol-3-yl)methoxy)phenyl)acetate (42b) (300 mg, 0.595 mmol) in dioxane (2 mL) and THF (2 mL) using 4-iodo-3-methoxypicolinonitrile (23b) (186 mg, 0.714 mmol), 2 M aqueous solution of K₃PO₄ (1.189 mL, 2.379 mmol), tricyclohexylphosphine (33.4 mg, 0.119 mmol), Pd₂(dba)₃ (54.5 mg, 0.059 mmol)), PdCl₂(dppf)-CH₂Cl₂ adduct (48.6 mg, 0.059 mmol) and heating at 100° C. for 2 h to afford after workup and purification using method-AC, ethyl 2-(2-((5-(2-cyano-3-methoxypyridin-4-yl)-1-cyclopentyl-1H-indazol-3-yl)methoxy)phenyl)acetate (327b) (200 mg, 66% yield) as a clear oil; ¹H NMR (300 MHz, DMSO-d₆) δ 8.55 (d, J=4.9 Hz, 1H), 8.08 (d, J=1.7, 0.8 Hz, 1H), 7.93-7.80 (m, 2H), 7.70 (dd, J=8.8, 1.6 Hz, 1H), 7.34-7.22 (m, 2H), 7.22-7.13 (m, 1H), 6.97-6.84 (m, 1H), 5.44 (s, 2H), 5.31-5.16 (m, 1H), 3.69 (q, J=7.1 Hz, 2H), 3.64 (s, 3H), 3.54 (s, 2H), 2.24-2.10 (m, 2H), 2.10-1.96 (m, 2H), 1.96-1.81 (m, 2H), 1.81-1.60 (m, 2H), 0.81 (t, J=7.1 Hz, 3H); MS (ES+): 511.20 (M+1); (ES−): 509.05 (M−1).

### Step-2: Preparation of ethyl 2-(2-((5-(2-(aminomethyl)-3-methoxypyridin-4-yl)-1-cyclopentyl-1H-indazol-3-yl)methoxy)phenyl)acetate (327c)

Compound 327e was prepared according to the procedure reported in scheme-287, from ethyl 2-(2-((5-(2-cyano-3-methoxypyridin-4-yl)-1-cyclopentyl-1H-indazol-3-yl)methoxy)phenyl)acetate (327b) (200 mg, 0.392 mmol) in anhydrous ethanol (10 mL) using nickel(II) chloride hexahydrate (23.28 mg, 0.098 mmol), sodium borohydride (44.5 mg, 1.175 mmol), N1-(2-aminoethyl)ethane-1,2-diamine (81 mg, 0.783 mmol) to afford after work up and purification using method-BI, ethyl 2-(2-((5-(2-(aminomethyl)-3-methoxypyridin-4-yl)-1-cyclopentyl-1H-indazol-3-yl)methoxy)phenyl)acetate (327c) (60 mg, 30% yield) as a white solid; ¹H NMR (300 MHz, DMSO-d₆) δ 8.35 (d, J=4.9 Hz, 1H), 8.02 (s, 1H), 7.83 (d, J=8.8 Hz, 1H), 7.69 (dd, J=8.8, 1.6 Hz, 1H), 7.36 (d, J=5.0 Hz, 1H), 7.27 (d, J=4.0 Hz, 2H), 7.18 (d, J=7.3 Hz, 1H), 6.95-6.86 (m, 1H), 5.43 (s, 2H), 5.29-5.15 (m, 1H), 3.90 (s, 2H), 3.67 (q, J=7.1 Hz, 2H), 3.53 (s, 2H), 3.36 (s, 3H), 2.22-2.11 (m, 2H), 2.09-1.99 (m, 2H), 1.94-1.84 (m, 2H), 1.79-1.65 (m, 2H), 0.80 (t, J=7.1 Hz, 3H); MS (ES+): 515.30 (M+1).

### Step-3: Preparation of 2-(2-((5-(2-(aminomethyl)-3-methoxypyridin-4-yl)-1-cyclopentyl-1H-indazol-3-yl)methoxy)phenyl)acetic acid (327d)

Compound 327d was prepared according to the procedure reported in step-2 of scheme-1, from ethyl 2-(2-((5-(2-(aminomethyl)-3-methoxypyridin-4-yl)-1-cyclopentyl-1H-indazol-3-yl)methoxy)phenyl)acetate (327c) (60 mg, 0.117 mmol) in THF (2 mL) and MeOH (2 mL) using a solution of lithium hydroxide hydrate (29.4 mg, 0.7 mmol) in water (1 mL) and stirring at RT for 15 h. This gave after workup and purification using method-G, 2-(2-((5-(2-(aminomethyl)-3-methoxypyridin-4-yl)-1-cyclopentyl-1H-indazol-3-yl)methoxy)phenyl)acetic acid (327d) (28 mg, 49% yield) HCl salt as a light yellow solid; H NMR (300 MHz, DMSO-d₆) δ 8.45 (d, J=5.0 Hz, 1H), 8.30 (s, 2H, D₂O exchangeable), 8.10 (s, 1H), 7.87 (d, J=8.8 Hz, 1H), 7.73-7.66 (m, 1H), 7.56 (d, J=5.0 Hz, 1H), 7.30-7.23 (m, 2H), 7.19 (d, J=7.2 Hz, 1H), 6.97-6.86 (m, 1H), 5.45 (s, 2H), 5.29-5.16 (m, 1H), 4.33-4.27 (m, 2H), 3.51 (s, 2H), 3.39 (s, 3H), 2.24-2.10 (m, 2H), 2.10-1.96 (m, 2H), 1.96-1.84 (m, 2H), 1.80-1.66 (m, 2H); MS (ES+): 487.20 (M+1); (ES−): 485.20 (M−1).

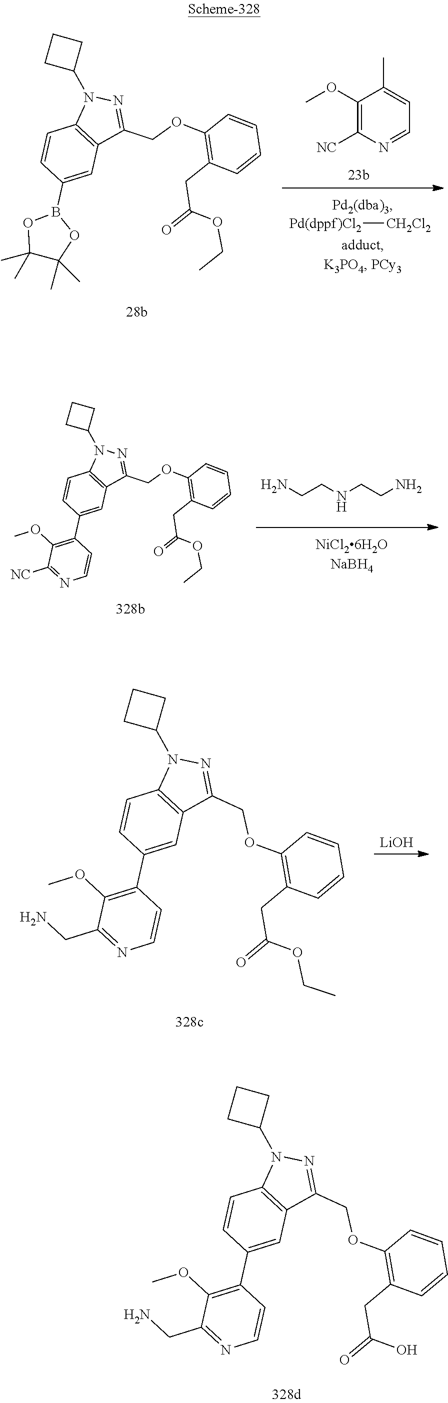

### Preparation of 2-(2-((5-(2-(aminomethyl)-3-methoxypyridin-4-yl)-1-cyclobutyl-1H-indazol-3-yl)methoxy)phenyl)acetic acid (328d)

### Step-1: Preparation of ethyl 2-(2-((5-(2-cyano-3-methoxypyridin-4-yl)-1-cyclobutyl-1H-indazol-3-yl)methoxy)phenyl)acetate (328b)

Compound 328b was prepared according to the procedure reported in step-5 of scheme 1, from ethyl 2-(2-((1-cyclobutyl-5-(4,4,5,5-tetramethyl-1,3,2-dioxaborolan-2-yl)-1H-indazol-3-yl)methoxy)phenyl)acetate (328a) (500 mg, 1.02 mmol) in dioxane (3 mL) and THF (3 mL) using 4-iodo-3-methoxypicolinonitrile (326a) (398 mg, 1.529 mmol), 2 M aqueous solution of K₃PO₄ (2.039 mL, 4.08 mmol), tricyclohexylphosphine (57.2 mg, 0.204 mmol), Pd₂(dba)₃ (93 mg, 0.102 mmol), PdCl₂(dppf)-CH₂Cl₂ adduct (83 mg, 0.102 mmol) and heating at 100° C. for 2 h to afford after workup and purification using method-AC, ethyl 2-(2-((5-(2-cyano-3-methoxypyridin-4-yl)-1-cyclobutyl-1H-indazol-3-yl)methoxy)phenyl)acetate (328b) (350 mg, 69% yield) as a clear oil; ¹H NMR (300 MHz, DMSO-d₆) δ 8.55 (d, J=4.9 Hz, 1H), 8.09 (d, J=1.7, 0.8 Hz, 1H), 7.90-7.80 (m, 2H), 7.70 (dd, J=8.9, 1.6 Hz, 1H), 7.32-7.24 (m, 2H), 7.22-7.15 (m, 1H), 6.96-6.86 (m, 1H), 5.46 (s, 2H), 5.43-5.27 (m, 1H), 3.68 (q, J=7.0 Hz, 2H), 3.63 (s, 3H), 3.54 (s, 2H), 2.77-2.58 (m, 2H), 2.50-2.42 (m, 2H), 1.97-1.82 (m, 2H), 0.81 (t, J=7.1 Hz, 3H); MS (ES+): 497.20 (M+1); (ES−): 495.05 (M−1).

### Step-2: Preparation of ethyl 2-(2-((5-(2-(aminomethyl)-3-methoxypyridin-4-yl)-1-cyclobutyl-1H-indazol-3-yl)methoxy)phenyl)acetate (328c)

Compound 328c was prepared according to the procedure reported in scheme-287, from ethyl 2-(2-((5-(2-cyano-3-methoxypyridin-4-yl)-1-cyclobutyl-1H-indazol-3-yl)methoxy)phenyl)acetate (328b) (350 mg, 0.705 mmol) in anhydrous ethanol (10 mL) using nickel(II) chloride hexahydrate (41.9 mg, 0.176 mmol), sodium borohydride (80 mg, 2.115 mmol), N1-(2-aminoethyl)ethane-1,2-diamine (145 mg, 1.41 mmol) to afford after work up and purification using method-BI, ethyl 2-(2-((5-(2-(aminomethyl)-3-methoxypyridin-4-yl)-1-cyclobutyl-1H-indazol-3-yl)methoxy)phenyl)acetate (328c) (100 mg, 28% yield) as a white solid; MS (ES+): 501.20 (M+1).

### Step-3: Preparation of 2-(2-((5-(2-(aminomethyl)-3-methoxypyridin-4-yl)-1-cyclobutyl-1H-indazol-3-yl)methoxy)phenyl)acetic acid (328d)

Compound 328d was prepared according to the procedure reported in step-2 of scheme-1, from ethyl 2-(2-((5-(2-(aminomethyl)-3-methoxypyridin-4-yl)-1-cyclobutyl-1H-indazol-3-yl)methoxy)phenyl)acetate (328c) (100 mg, 0.200 mmol) in THF (2 mL) and MeOH (2 mL) using a solution of lithium hydroxide hydrate (50.3 mg, 1.199 mmol) in water (1 mL) and stirring at RT for 15 h to afford after workup and purification using method-G, 2-(2-((5-(2-(aminomethyl)-3-methoxypyridin-4-yl)-1-cyclobutyl-1H-indazol-3-yl)methoxy)phenyl)acetic acid (328d) (35 mg, 37% yield) HCl salt as a yellow solid; ¹H NMR (300 MHz, DMSO-d₆) δ 8.52-8.45 (m, 2H, D₂O exchangeable), 8.44 (d, J=5.3 Hz, 1H), 8.10 (s, 1H), 7.85 (d, J=8.8 Hz, 1H), 7.70 (d, J=8.8 Hz, 1H), 7.55 (d, J=5.1 Hz, 1H), 7.27 (d, J=4.2 Hz, 2H), 7.20 (d, J=7.3 Hz, 1H), 6.98-6.85 (m, 1H), 5.48 (s, 2H), 5.41-5.27 (m, 1H), 4.33-4.23 (m, 2H), 3.52 (s, 2H), 3.37 (s, 3H), 2.76-2.58 (m, 2H), 2.58-2.51 (m, 2H), 2.01-1.80 (m, 2H); MS (ES+): 473.20 (M+1); (ES−): 471.20 (M−1); Analysis calculated for C₂₇H₂₈N₄O₄·2.25HCl·2.75H₂O: C, 53.68; H, 5.97; N, 9.27; Found: C, 53.73; H, 5.79; N, 9.29.

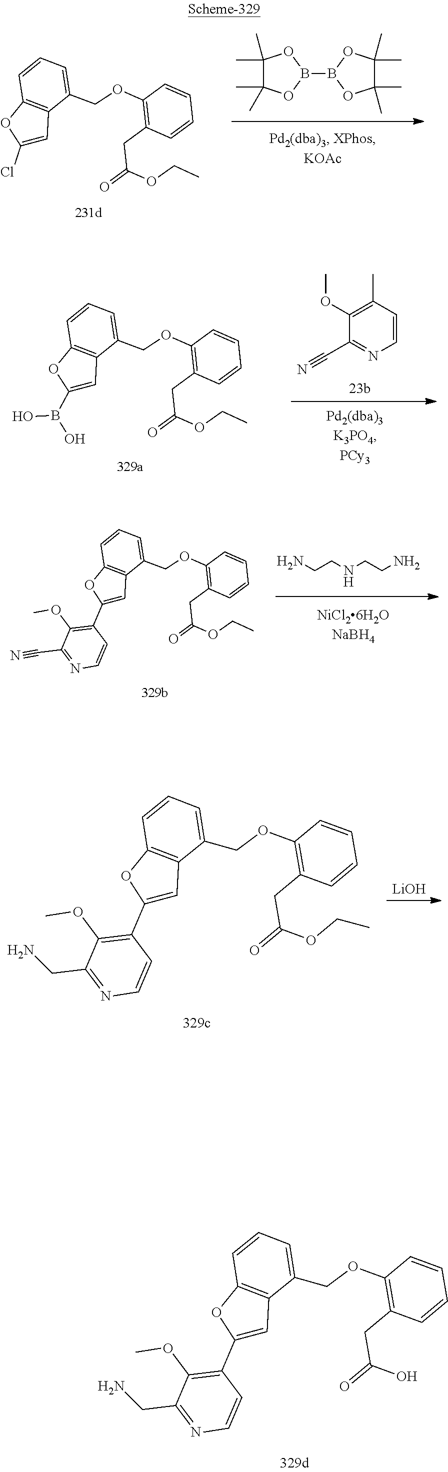

### Preparation of 2-(2-((2-(2-(aminomethyl)-3-methoxypyridin-4-yl)benzofuran-4-yl)methoxy)phenyl)acetic acid (329d)

### Step-1: Preparation of (4-((2-(2-ethoxy-2-oxoethyl)phenoxy)methyl)benzofuran-2-yl)boronic acid (329a)

A mixture of ethyl 2-(2-((2-chlorobenzofuran-4-yl)methoxy)phenyl)acetate (231d) (200 mg, 0.580 mmol), BISPIN (295 mg, 1.160 mmol), KOAc (228 mg, 2.320 mmol), X-Phos (55.3 mg, 0.116 mmol) and Pd₂(dba)₃ (53.1 mg, 0.058 mmol) in dry dioxane (10 mL) was heated to 90° C. under argon for 24 h. The solvent was removed in vacuo. The residue obtained was purified using method-BG to afford 4-((2-(2-ethoxy-2-oxoethyl)phenoxy)methyl)benzofuran-2-ylboronic acid (329a) (205 mg, >99% yield) as a dark oil; MS (ES+): 377.1 (M+Na); (ES−): 389.0 (M+Cl).

### Step-2: Preparation of ethyl 2-(2-((2-(2-cyano-3-methoxypyridin-4-yl)benzofuran-4-yl)methoxy)phenyl)acetate (329b)

Compound 329b was prepared according to the procedure reported in step-3 of scheme-112, from (4-((2-(2-ethoxy-2-oxoethyl)phenoxy)methyl)benzofuran-2-yl)boronic acid (329a) (205 mg, 0.579 mmol) in dioxane (6 mL) and 2Me-THF (3 mL) using 4-iodo-3-methoxypicolinonitrile (326a) (176 mg, 0.677 mmol), Pd₂(dba)₃ (106 mg, 0.116 mmol), tripotassium phosphate (4M aqueous, 0.579 mL, 2.315 mmol), tricyclohexylphosphine (64.9 mg, 0.232 mmol) and heating at 115° C. for 4.5 h to afford after workup and purification using method-AQ, ethyl 2-(2-((2-(2-cyano-3-methoxypyridin-4-yl)benzofuran-4-yl)methoxy)phenyl)acetate (329b) (100 mg, 39.0% yield) as a yellow oil; MS (ES+): 443.1 (M+H).

### Step-3: Preparation of ethyl 2-(2-((2-(2-(aminomethyl)-3-methoxypyridin-4-yl)benzofuran-4-yl)methoxy)phenyl)acetate (329c)

Compound 329c was prepared according to the procedure reported in scheme-287, from ethyl 2-(2-((2-(2-cyano-3-methoxypyridin-4-yl)benzofuran-4-yl)methoxy)phenyl)acetate (329b) (100 mg, 0.226 mmol) in anhydrous methanol (10 mL) using nickel(II) chloride hexahydrate (21.49 mg, 0.090 mmol), sodium borohydride (90 mg, 2.379 mmol). N1-(2-aminoethyl)ethane-1,2-diamine (0.073 mL, 0.678 mmol) to afford after workup and purification using method-BP, ethyl 2-(2-((2-(2-(aminomethyl)-3-methoxypyridin-4-yl)benzofuran-4-yl)methoxy)phenyl)acetate (329c) (51 mg, 51% yield) as a yellow oil; MS (ES+): 447.2 (M+1).

### Step-4: Preparation of 2-(2-((2-(2-(aminomethyl)-3-methoxypyridin-4-yl)benzofuran-4-yl)methoxy)phenyl)acetic acid (329d)

Compound 329d was prepared according to the procedure reported in step-2 of scheme-1, from ethyl 2-(2-((2-(2-(aminomethyl)-3-methoxypyridin-4-yl)benzofuran-4-yl)methoxy)phenyl)acetate (329c) (51 mg, 0.114 mmol) in MeOH/THF (3 mL each) using a solution of lithium hydroxide monohydrate (40 mg, 0.953 mmol) in water (2 mL) and stirring at RT for 5 h. This gave after workup and purification using method-G, 2-(2-((2-(2-(aminomethyl)-3-methoxypyridin-4-yl)benzofuran-4-yl)methoxy)phenyl)acetic acid (329d) (42 mg, 88% yield) HCl salt as a yellow solid; ¹H NMR (300 MHz, DMSO-d₆) δ 8.72-8.43 (m, 4H, 3H D₂O exchangeable), 7.95 (d, J=5.1 Hz, 1H), 7.85 (s, 1H), 7.69 (dd, J=6.9, 2.2 Hz, 1H), 7.59-7.42 (m, 2H), 7.33-7.15 (m, 3H), 6.93 (td, J=7.3, 1.4 Hz, 1H), 5.49 (s, 2H), 4.41-4.28 (m, 2H), 3.87 (s, 3H), 3.60 (s, 2H); MS (ES+): 419.10 (M+1); MS (ES−): 417.10 (M−1).

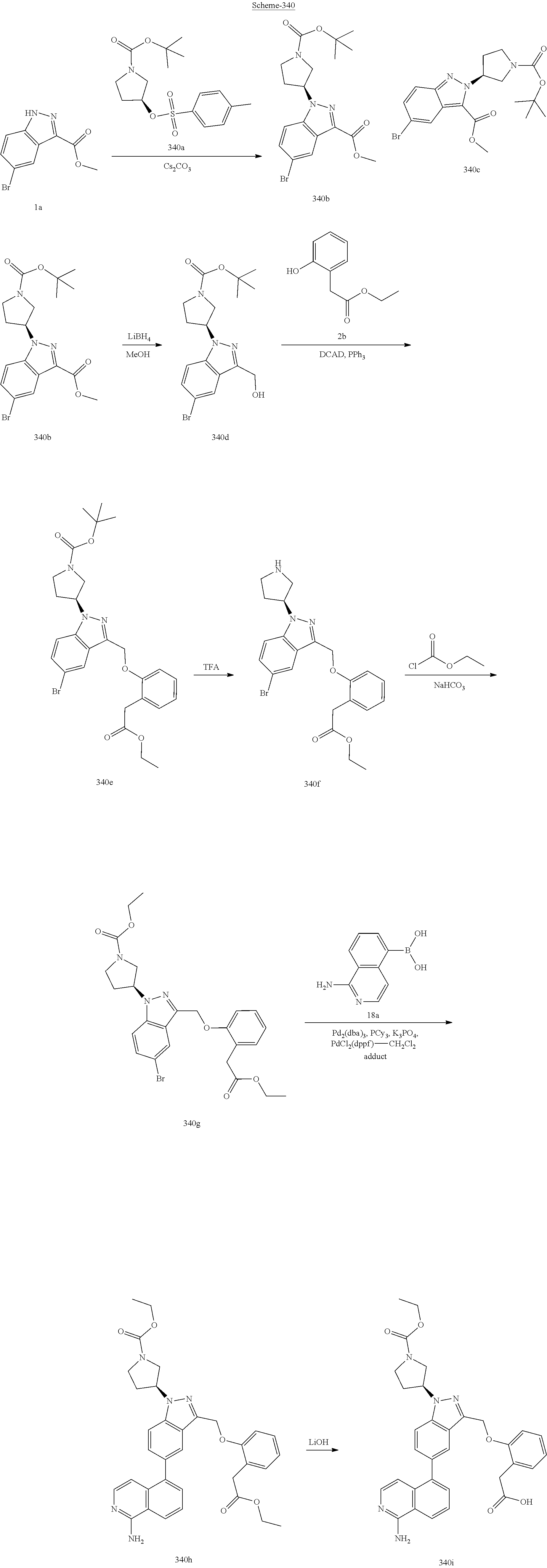

### Preparation of (R)-2-(2-((5-(1-aminoisoquinolin-5-yl)-1-(1-(ethoxycarbonyl)pyrrolidin-3-yl)-1H-indazol-3-yl)methoxy)phenyl)acetic acid (340i)

### Step-1: Preparation of (R)-methyl 5-bromo-1-(1-(tert-butoxycarbonyl)pyrrolidin-3-yl)-1H-indazole-3-carboxylate (340b) and (R)-methyl 5-bromo-2-(1-(tert-butoxycarbonyl)pyrrolidin-3-yl)-2H-indazole-3-carboxylate (340c)

Compound 340b was prepared according to the procedure reported in step-2 of scheme-86, from methyl 5-bromo-1H-indazole-3-carboxylate (1a) (23.16 g, 91 mmol) in DMF (20 mL) using Cs₂CO₃ (59.2 g, 182 mmol), (S)-tert-butyl 3-(tosyloxy)pyrrolidine-1-carboxylate (340a) (31 g, 91 mmol; CAS #371240-55-0) and stirring at 80° C. for 2 h to afford after work up and purification using method-AL (R)-methyl 5-bromo-1-(1-(tert-butoxycarbonyl)pyrrolidin-3-yl)-1H-indazole-3-carboxylate (340b) (13.8 g, 36% yield) as a white solid and (R)-methyl 5-bromo-2-(1-(tert-butoxycarbonyl)pyrrolidin-3-yl)-2H-indazole-3-carboxylate (340c) (3.2 g, 8% yield) as a white solid; MS (ES+): 446.1 (M+Na).

### Step-2: Preparation of (R)-tert-butyl 3-(5-bromo-3-(hydroxymethyl)-1H-indazol-1-yl)pyrrolidine-1-carboxylate (340d)

Compound 340d was prepared according to the procedure reported in step-2 of scheme-119, from (R)-methyl 5-bromo-1-(1-(tert-butoxycarbonyl)pyrrolidin-3-yl)-1H-indazole-3-carboxylate (340b) (13.5 g, 31.8 mmol) in THF (250 mL) using LiBH₄ (2 M solution of THF, 15.91 mL, 31.8 mmol) and MeOH (3.22 mL, 80 mmol) to afford after work up and purification using method-BQ (R)-tert-butyl 3-(5-bromo-3-(hydroxymethyl)-1H-indazol-1-yl)pyrrolidine-1-carboxylate (340d) (9.8 g, 78% yield) as a clear oil; MS (ES+): 418.1 (M+Na).

### Step-3: Preparation of (R)-tert-butyl 3-(5-bromo-3-((2-(2-ethoxy-2-oxoethyl)phenoxy)methyl)-1H-indazol-1-yl)pyrrolidine-1-carboxylate (340e)

Compound 340e was prepared according to the procedure reported in step-2 of scheme-2, from (R)-tert-butyl 3-(5-bromo-3-(hydroxymethyl)-1H-indazol-1-yl)pyrrolidine-1-carboxylate (340d) (1.5 g, 3.79 mmol) in DCM (25 mL) using ethyl 2-(2-hydroxyphenyl)acetate (2b) (0.819 g, 4.54 mmol), PPh₃ (1.092 g, 4.16 mmol) and a solution of DCAD (1.529 g, 4.16 mmol) in DCM (5 mL) to afford after workup and purification using method-BR. (R)-tert-butyl 3-(5-bromo-3-((2-(2-ethoxy-2-oxoethyl)phenoxy)methyl)-1H-indazol-1-yl)pyrrolidine-1-carboxylate (340e) (1.1 g, 52% yield) as a white solid. MS (ES+): 558.1 (M+1).

### Step-4: Preparation of (R)-ethyl 2-(2-((5-bromo-1-(pyrrolidin-3-yl)-1H-indazol-3-yl)methoxy)phenyl)acetate (340f)

Compound 340f was prepared according to the procedure reported in step-4 of scheme-9, from (R)-tert-butyl 3-(5-bromo-3-((2-(2-ethoxy-2-oxoethyl)phenoxy)methyl)-1H-indazol-1-yl)pyrrolidine-1-carboxylate (340e) (1 g, 1.791 mmol) in DCM (100 mL) using TFA (1.380 mL, 17.91 mmol) and stirring at RT for 16 h. The reaction mixture was concentrated and the residue was neutralized with sodium bicarbonate, extracted with ethyl acetate (2×50 mL), washed with water (100 mL), brine (50 mL) dried and concentrated in vacuum to afford (R)-ethyl 2-(2-((5-bromo-1-(pyrrolidin-3-yl)-1H-indazol-3-yl)methoxy)phenyl)acetate (340f) (742 mg, 90% yield) and was used as such without further purification; MS (ES+): 458.1 (M+1).

### Step-5: Preparation of (R)-ethyl 3-(5-bromo-3-((2-(2-ethoxy-2-oxoethyl)phenoxy)methyl)-1H-indazol-1-yl)pyrrolidine-1-carboxylate (340g)

Compound 340g was prepared according to the procedure reported in step-1 of scheme-126, from (R)-ethyl 2-(2-((5-bromo-1-(pyrrolidin-3-yl)-1H-indazol-3-yl)methoxy)phenyl)acetate (340f) (905 mg, 1.974 mmol) in THF (5 mL) and water (5 mL) using sodium bicarbonate (0.249 g, 2.96 mmol) and ethyl carbonochloridate (0.321 g, 2.96 mmol) to afford after workup and purification using method-J. (R)-ethyl 3-(5-bromo-3-((2-(2-ethoxy-2-oxoethyl)phenoxy)methyl)-1H-indazol-1-yl)pyrrolidine-1-carboxylate (340g) (743 mg, 71% yield) as a clear oil; MS (ES+): 530.1 (M+1).

### Step-6: Preparation of (R)-ethyl 3-(5-(1-aminoisoquinolin-5-yl)-3-((2-(2-ethoxy-2-oxoethyl)phenoxy) methyl)-1H-indazol-1-yl)pyrrolidine-1-carboxylate (340h)

Compound 340h was prepared according to the procedure reported in step-5 of scheme-1, from (R)-ethyl 3-(5-bromo-3-((2-(2-ethoxy-2-oxoethyl)phenoxy)methyl)-1H-indazol-1-yl)pyrrolidine-1-carboxylate (340g) (200 mg, 0.377 mmol) in dioxane/MeTHF (10 mL, ratio 9:1) using 1-aminoisoquinolin-5-ylboronic acid (18a) (92 mg, 0.490 mmol), K₃PO₄ (2M aqueous solution, 754 μl, 1.508 mmol). PCy₃ (21.15 mg, 0.075 mmol), Pd₂(dba)₃ (34.5 mg, 0.038 mmol) and PdCl₂(dppf)-CH₂Cl₂Adduct (30.8 mg, 0.038 mmol) to afford after workup and purification using method-AE, (R)-ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (340h) (142 mg, 63% yield) as a yellow solid; MS (ES+): 594.3 (M+1).

### Step-7: Preparation of (R)-2-(2-((5-(1-aminoisoquinolin-5-yl)-1-(1-(ethoxycarbonyl)pyrrolidin-3-yl)-1H-indazol-3-yl)methoxy)phenyl)acetic acid (340i)

Compound 340i was prepared according to the procedure reported in step-2 of scheme-1, from (R)-ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (340h) (140 mg, 0.236 mmol) in THF (3 mL) using lithium hydroxide hydrate (29.7 mg, 0.707 mmol) in water (1 mL) and stirring overnight at RT to afford after work up and purification using method-M. (R)-2-(2-((5-(1-aminoisoquinolin-5-yl)-1-(1-(ethoxycarbonyl)pyrrolidin-3-yl)-1H-indazol-3-yl)methoxy)phenyl)acetic acid (340i) (46.0 mg, 35% yield) HCl salt as a white solid; ¹H NMR (300 MHz, DMSO-d₆) δ 13.21 (s, 1H, D₂O Exchangeable), 11.99 (s, 1H, D₂O Exchangeable), 9.08 (s, 2H, D₂O Exchangeable), 8.61 (d, J=8.3 Hz, 1H), 7.99-7.79 (m, 4H), 7.62 (d, J=7.2 Hz, 1H), 7.51 (dd, J=8.6, 1.7 Hz, 1H), 7.29-7.20 (m, 2H), 7.20-7.12 (m, 1H), 6.98 (d, J=7.2 Hz, 1H), 6.94-6.84 (m, 1H), 5.59-5.53 (m, 1H), 5.45 (s, 2H), 4.10-4.04 (m, 2H), 3.94-3.85 (m, 1H), 3.77-3.65 (m, 1H), 3.68-3.62 (m, 1H), 3.57-3.51 (m, 1H), 3.47 (s, 2H), 2.49-2.39 (m, 2H), 1.28-1.13 (m, 3H); MS (ES+): 566.3 (M+1); (ES−): 564.3 (M−1); Optical Rotation: +11.852 [0.135, MeOH].

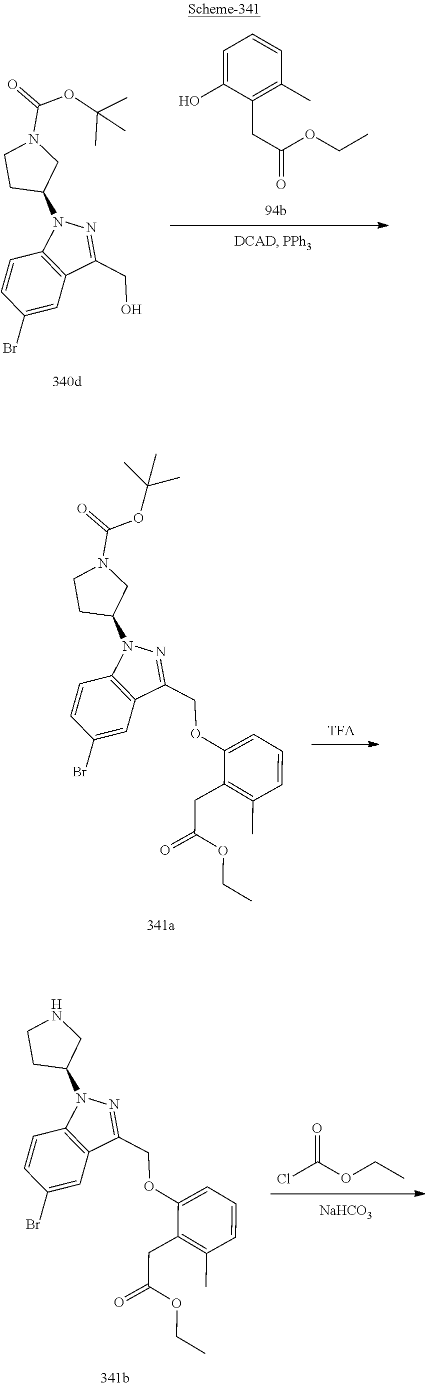

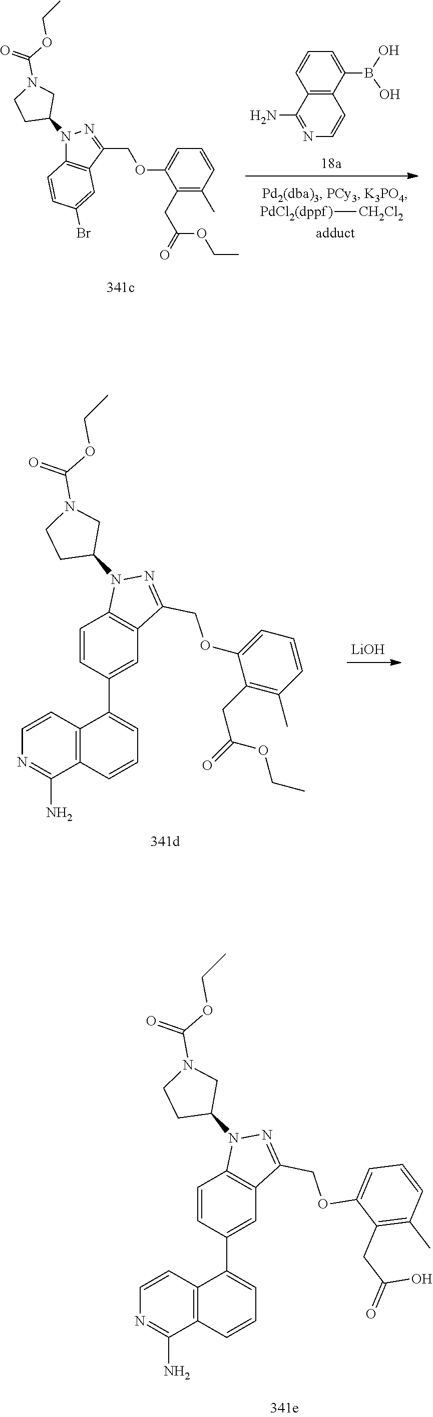

### Preparation of (R)-2-(2-((5-(1-aminoisoquinolin-5-yl)-1-(1-(ethoxycarbonyl)pyrrolidin-3-yl)-1H-indazol-3-yl)methoxy)-6-methylphenyl)acetic acid (341e)

### Step-1: Preparation of (R)-tert-butyl 3-(5-bromo-3-((2-(2-ethoxy-2-oxoethyl)-3-methylphenoxy)methyl)-1H-indazol-1-yl)pyrrolidine-1-carboxylate (341a)

Compound 341a was prepared according to the procedure reported in step-2 of scheme-2, from (R)-tert-butyl 3-(5-bromo-3-(hydroxymethyl)-1H-indazol-1-yl)pyrrolidine-1-carboxylate (340d) (1.5 g, 3.79 mmol) in DCM (25 mL) using ethyl 2-(2-hydroxy-6-methylphenyl)acetate (94b) (0.882 g, 4.54 mmol), PPh₃ (1.092 g, 4.16 mmol) and a solution of DCAD (1.529 g, 4.16 mmol) in DCM (5 mL) to afford after workup and purification using method-BR, (R)-tert-butyl 3-(5-bromo-3-((2-(2-ethoxy-2-oxoethyl)-3-methylphenoxy)methyl)-1H-indazol-1-yl)pyrrolidine-1-carboxylate (341a) (1.41 g, 65% yield) as a white solid; MS (ES+): 572.2 (M+1).

### Step-2: Preparation of (R)-ethyl 2-(2-((5-bromo-1-(pyrrolidin-3-yl)-1H-indazol-3-yl)methoxy)-6-methylphenyl)acetate (341b)

Compound 341b was prepared according to the procedure reported in step-4 of scheme-9, from (R)-tert-butyl 3-(5-bromo-3-((2-(2-ethoxy-2-oxoethyl)-3-methylphenoxy)methyl)-1H-indazol-1-yl)pyrrolidine-1-carboxylate (341a) (1.31 g, 2.288 mmol) in DCM (100 mL) using TFA (1.763 mL, 22.88 mmol) and stirring at RT for 16 h. The reaction mixture was concentrated and the residue was neutralized with sodium bicarbonate and was extracted with ethyl acetate (2×50 mL), washed with water (100 mL), brine (50 mL) dried and concentrated to afford (R)-ethyl 2-(2-((5-bromo-1-(pyrrolidin-3-yl)-1H-indazol-3-yl)methoxy)-6-methylphenyl)acetate (341b) (1.02 g, 94% yield) and was used as such without further purification; MS (ES+): 472.2 (M+1).

### Step-3: Preparation of (R)-ethyl 3-(5-bromo-3-((2-(2-ethoxy-2-oxoethyl)-3-methylphenoxy)methyl)-1H-indazol-1-yl)pyrrolidine-1-carboxylate (341c)

Compound 341c was prepared according to the procedure reported in step-1 of scheme-126, from

(R)-ethyl 2-(2-((5-bromo-1-(pyrrolidin-3-yl)-1H-indazol-3-yl)methoxy)-6-methylphenyl)acetate (341b) (1.01 g, 2.138 mmol) in THF (5 mL) and water (5 mL) using sodium bicarbonate (0.269 g, 3.21 mmol) and ethyl carbonochloridate (0.348 g, 3.21 mmol) to afford after workup and purification using method-J, (R)-ethyl 3-(5-bromo-3-((2-(2-ethoxy-2-oxoethyl)-3-methylphenoxy)methyl)-1H-indazol-1-yl)pyrrolidine-1-carboxylate (341c) (841 mg, 72% yield) as a clear oil; MS (ES+): 544.2 (M+1).

### Step-4: Preparation of (R)-ethyl 3-(5-(1-aminoisoquinolin-5-yl)-3-((2-(2-ethoxy-2-oxoethyl)-3-methylphenoxy)methyl)-1H-indazol-1-yl)pyrrolidine-1-carboxylate (341d)

Compound 341d was prepared according to the procedure reported in step-5 of scheme-1, from (R)-ethyl 3-(5-bromo-3-((2-(2-ethoxy-2-oxoethyl)-3-methylphenoxy)methyl)-1H-indazol-1-yl)pyrrolidine-1-carboxylate (341c) (200 mg, 0.367 mmol) in dioxane/MeTHF (10 mL, ratio 9:1) using 1-aminoisoquinolin-5-ylboronic acid (18a) (90 mg, 0.478 mmol), K₃PO₄ (2M aqueous solution, 735 μl, 1.469 mmol), PCy₃ (20.60 mg, 0.073 mmol), Pd₂(dba)₃ (33.6 mg, 0.037 mmol) and PdCl₂(dppf)-CH₂Cl₂Adduct (30.0 mg, 0.037 mmol) to afford after workup and purification using method-AE, (R)-ethyl 3-(5-(1-aminoisoquinolin-5-yl)-3-((2-(2-ethoxy-2-oxoethyl)-3-methylphenoxy)methyl)-1H-indazol-1-yl)pyrrolidine-1-carboxylate (341d) (141 mg, 63% yield) as a yellow solid; MS (ES+): 608.3 (M+1).

### Step-5: Preparation of (R)-2-(2-((5-(1-aminoisoquinolin-5-yl)-1-(1-(ethoxycarbonyl)pyrrolidin-3-yl)-1H-indazol-3-yl)methoxy)-6-methylphenyl)acetic acid (341e)

Compound 341e was prepared according to the procedure reported in step-2 of scheme-1, from (R)-ethyl 3-(5-(1-aminoisoquinolin-5-yl)-3-(2-(2-ethoxy-2-oxoethyl)-3-methylphenoxy)methyl)-1H-indazol-1-yl)pyrrolidine-1-carboxylate (341d) (135 mg, 0.222 mmol) in THF (3 mL) using lithium hydroxide hydrate (28.0 mg, 0.666 mmol) in water (1 mL) and stirring overnight at RT to afford after work up and purification using method-M, (R)-2-(2-((5-(1-aminoisoquinolin-5-yl)-1-(1-(ethoxycarbonyl)pyrrolidin-3-yl)-1H-indazol-3-yl)methoxy)-6-methylphenyl)acetic acid (341e) (63.6 mg, 49% yield) HCl salt as a white solid; ¹H NMR (300 MHz, DMSO-d₆) δ 13.20 (s, 1H, D₂O exchangeable), 12.00 (s, 1H, D₂O exchangeable), 9.15 (s, 2H, D₂O exchangeable), 8.62 (d, J=8.2 Hz, 1H), 7.99-7.80 (m, 4H), 7.63 (d, J=7.2 Hz, 1H), 7.51 (dd, J=8.7, 1.6 Hz, 1H), 7.17-7.05 (m, 2H), 6.97 (d, J=7.2 Hz, 1H), 6.85-6.74 (m, 1H), 5.60-5.52 (m, 1H), 5.42 (s, 2H), 4.12-4.02 (m, 2H), 3.93-3.87 (m, 1H), 3.76-3.60 (m, 2H), 3.60-3.46 (m, 3H), 2.47-2.32 (m, 2H), 2.17 (s, 3H), 1.32-1.09 (m, 3H); MS (ES+): 580.3 (M+1); (ES−): 578.2 (M−1); Optical Rotation: +10.286 [0.175, MeOH].

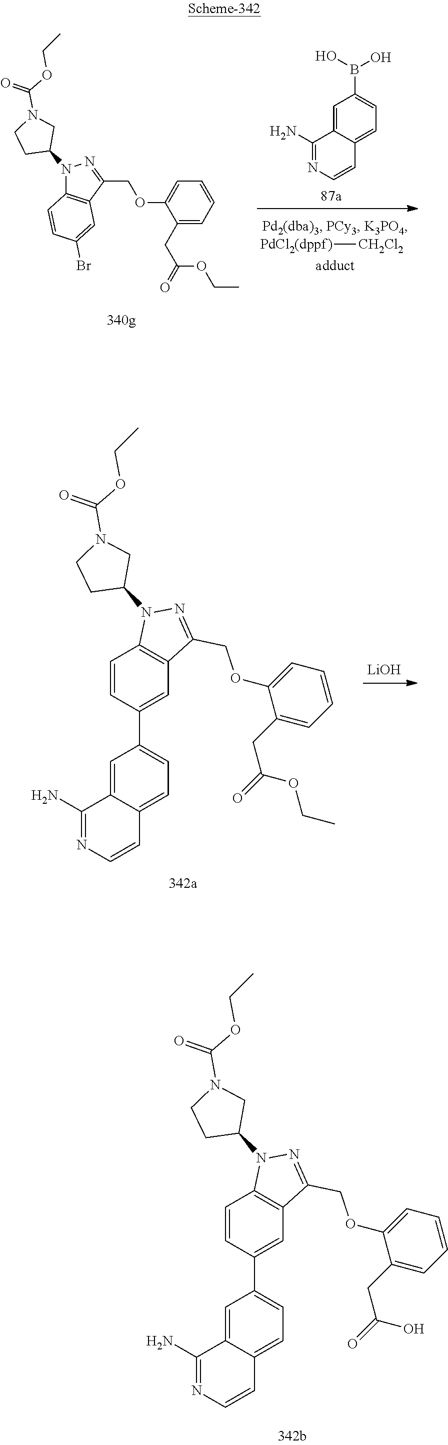

### Preparation of (R)-2-(2-((5-(1-aminoisoquinolin-7-yl)-1-(1-(ethoxycarbonyl)pyrrolidin-3-yl)-1H-indazol-3-yl)methoxy)phenyl)acetic acid (342b)

### Step-1: Preparation of (R)-ethyl 3-(5-(1-aminoisoquinolin-7-yl)-3-((2-(2-ethoxy-2-oxoethyl)phenoxy)methyl)˜1H-indazol-1-yl)pyrrolidine-1-carboxylate (342a)

Compound 342a was prepared according to the procedure reported in step-5 of scheme-1, from (R)-ethyl 3-(5-bromo-3-((2-(2-ethoxy-2-oxoethyl)phenoxy)methyl)-1H-indazol-1-yl)pyrrolidine-1-carboxylate (340g) (200 mg, 0.377 mmol) in dioxane/MeTHF (10 mL, ratio 9:1) using (1-aminoisoquinolin-7-yl)boronic acid (87a) (92 mg, 0.49 mmol), K₃PO₄ (4M aqueous solution, 377 μl, 1.508 mmol), PCy₃ (21 mg, 0.075 mmol), Pd₂(dba)₃ (35 mg, 0.038 mmol) and PdCl₂(dppf)-CH₂Cl₂Adduct (31 mg, 0.038 mmol) to afford after workup and purification using method-AE, (R)-ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (342a) (118 mg, 53% yield) as a yellow solid; MS (ES+): 594.3 (M+1).

### Step-2: Preparation of (R)-2-(2-((5-(1-aminoisoquinolin-7-yl)-1-(1-(ethoxycarbonyl)pyrrolidin-3-yl)-1H-indazol-3-yl)methoxy)phenyl)acetic acid (342b)

Compound 342b was prepared according to the procedure reported in step-2 of scheme-1, from (R)-ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (342a) (115 mg, 0.194 mmol) in THF (3 mL) using lithium hydroxide hydrate (24.38 mg, 0.581 mmol) in water (1 mL) and stirring overnight at RT to afford after work up and purification using method-M, (R)-2-(2-((5-(1-aminoisoquinolin-7-yl)-1-(1-(ethoxycarbonyl)pyrrolidin-3-yl)-1H-indazol-3-yl)methoxy)phenyl)acetic acid (342b) (34.7 mg, 32% yield) HCl salt as a white solid; ¹H NMR (300 MHz, DMSO-d₆) δ 13.05 (s, 1H, D₂O Exchangeable), 12.06 (s, 1H, D₂O Exchangeable), 9.03 (s, 2H, D₂O Exchangeable), 8.94 (d, J=1.6 Hz, 1H), 8.41 (dd, J=8.5, 1.6 Hz, 1H), 8.32 (d, J=1.5 Hz, 1H), 8.08-7.99 (m, 2H), 7.98-7.91 (m, 1H), 7.69 (d, J=6.9 Hz, 1H), 7.33-7.23 (m, 3H), 7.23-7.15 (m, 1H), 6.96-6.87 (m, 1H), 5.61-5.50 (m, 1H), 5.48 (s, 2H), 4.07 (t, J=7.0 Hz, 2H), 3.94-3.81 (m, 1H), 3.78-3.62 (m, 2H), 3.60-3.46 (m, 3H), 2.48-2.31 (m, 2H), 1.29-1.12 (m, 3H); MS (ES+): 566.3 (M+1); (ES−): 564.3 (M−1); Optical Rotation: −13.846 [0.130, MeOH].

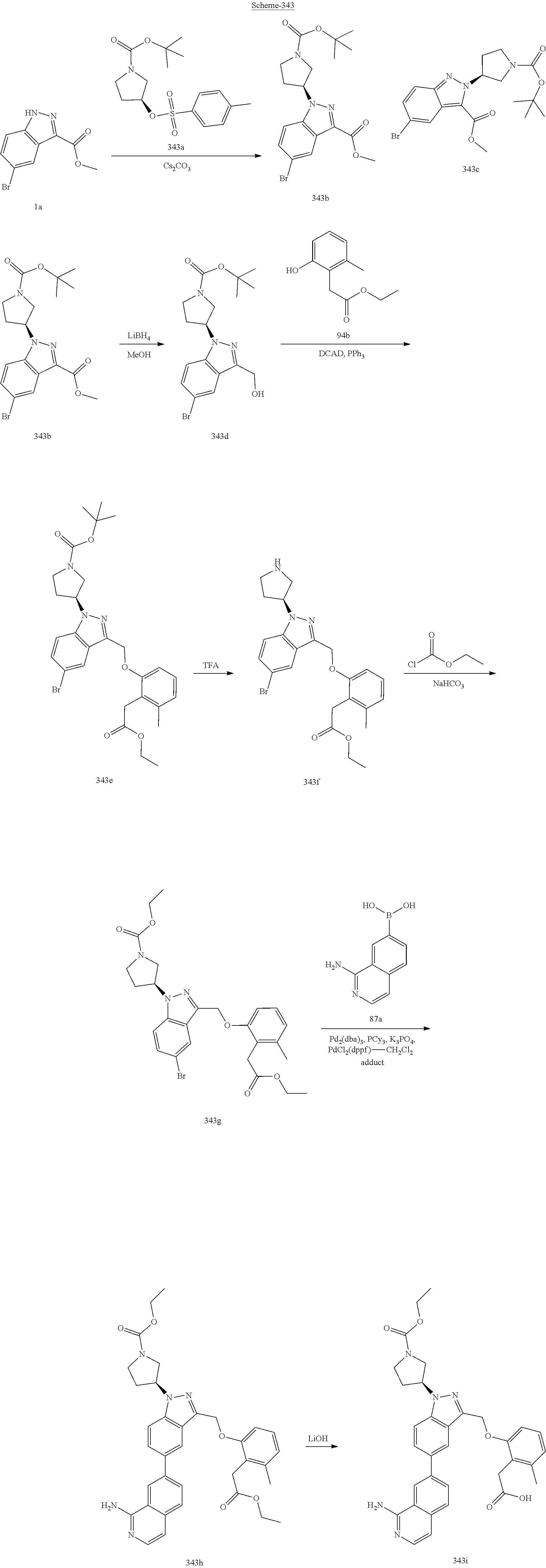

### Preparation of (S)-2-(2-((5-(1-aminoisoquinolin-7-yl)-1-(1-(ethoxycarbonyl)pyrrolidin-3-yl)-1H-indazol-3-yl)methoxy)-6-methylphenyl)acetic acid (343i)

### Step-1: Preparation of (S)-methyl 5-bromo-1-(1-(tert-butoxycarbonyl)pyrrolidin-3-yl)-1H-indazole-3-carboxylate (343b) and (S)-methyl 5-bromo-2-(1-(tert-butoxycarbonyl)pyrrolidin-3-yl)-2H-indazole-3-carboxylate (343c)

Compound 343b was prepared according to the procedure reported in step-2 of scheme-86, from methyl 5-bromo-1H-indazole-3-carboxylate (1a) (27.9 g, 110 mmol) in DMF (150 mL) using Cs₂CO₃ (71.4 g, 219 mmol), (R)-tert-butyl 3-(tosyloxy)pyrrolidine-1-carboxylate (343a) (37.4 g, 110 mmol; CAS #139986-03-1) and stirring at 80° C. for 2 h to afford after work up and purification using method-AL, (S)-methyl 5-bromo-1-(1-(tert-butoxycarbonyl)pyrrolidin-3-yl)-1H-indazole-3-carboxylate (343b) (14.7 g, 32% yield) as a white solid; MS (ES+): 446.1 (M+Na), and (S)-methyl 5-bromo-2-(1-(tert-butoxycarbonyl)pyrrolidin-3-yl)-2H-indazole-3-carboxylate (343c) (4.3 g, 9% yield) as a white solid; MS (ES+): 446.1 (M+Na).

### Step-2: Preparation of (S)-tert-butyl 3-(5-bromo-3-(hydroxymethyl)-1H-indazol-1-yl)pyrrolidine-1-carboxylate (343d)

Compound 343d was prepared according to the procedure reported in step-2 of scheme-119, from (S)-methyl 5-bromo-1-(1-(tert-butoxycarbonyl)pyrrolidin-3-yl)-1H-indazole-3-carboxylate (343b) (14.7 g, 34.6 mmol) in THF (250 mL) using LiBH₄ (2 M solution of THF, 17.32 mL, 34.6 mmol) and MeOH (3.5 mL, 87 mmol) to afford after work up and purification using method-BQ, (S)-tert-butyl 3-(5-bromo-3-(hydroxymethyl)-1H-indazol-1-yl)pyrrolidine-1-carboxylate (343d) (10.3 g, 75% yield) as a clear oil; MS (ES+): 418.1 (M+Na).

### Step-3: Preparation of (S)-tert-butyl 3-(5-bromo-3-((2-(2-ethoxy-2-oxoethyl)-3-methylphenoxy)methyl)-1H-indazol-1-yl)pyrrolidine-1-carboxylate (343e)

Compound 343e was prepared according to the procedure reported in step-2 of scheme-2, from (S)-tert-butyl 3-(5-bromo-3-(hydroxymethyl)-1H-indazol-1-yl)pyrrolidine-1-carboxylate (343d) (1.5 g, 3.79 mmol) in DCM (25 mL) using ethyl 2-(2-hydroxy-6-methylphenyl)acetate (94b) (0.882 g, 4.54 mmol). PPh₃ (1.092 g, 4.16 mmol) and a solution of DCAD (1.529 g, 4.16 mmol) in DCM (5 mL) to afford after workup and purification using method-BR, (S)-tert-butyl 3-(5-bromo-3-((2-(2-ethoxy-2-oxoethyl)-3-methylphenoxy)methyl)-1H-indazol-1-yl)pyrrolidine-1-carboxylate (343e) (1.32 g, 61% yield) as a white solid; MS (ES+): 572.2 (M+1).

### Step-4: Preparation of (S)-ethyl 2-(2-((5-bromo-1-(pyrrolidin-3-yl)-1H-indazol-3-yl)methoxy)-6-methylphenyl)acetate (343f)

Compound 343f was prepared according to the procedure reported in step-4 of scheme-9, from (S)-tert-butyl 3-(5-bromo-3-((2-(2-ethoxy-2-oxoethyl)-3-methylphenoxy)methyl)-1H-indazol-1-yl)pyrrolidine-1-carboxylate (343e) (1.31 g, 2.288 mmol) in DCM (100 mL) using TFA (1.763 mL, 22.88 mmol) and stirring at RT for 16 h. The reaction mixture was concentrated and the residue was neutralized with sodium bicarbonate and was extracted with ethyl acetate (2×50 mL), washed with water (100 mL), brine (50 mL) dried and concentrated to afford (S)-ethyl 2-(2-((5-bromo-1-(pyrrolidin-3-yl)-1H-indazol-3-yl)methoxy)-6-methylphenyl)acetate (343f) (1.06 g, 98% yield) and was used as such without further purification; MS (ES+): 472.2 (M+1).

### Step-5: Preparation of 3-(5-bromo-3-((2-(2-ethoxy-2-oxoethyl)-3-(S)-ethyl methylphenoxy)methyl)-1H-indazol-1-yl)pyrrolidine-1-carboxylate (343g)

Compound 343g was prepared according to the procedure reported in step-1 of scheme-126, from (S)-ethyl 2-(2-((5-bromo-1-(pyrrolidin-3-yl)-1H-indazol-3-yl)methoxy)-6-methylphenyl)acetate (343f) (740 mg, 1.567 mmol) in THF (5 mL) and water (5 mL) using sodium bicarbonate (197 mg, 2.35 mmol) and ethyl carbonochloridate (213 μl, 2.35 mmol) to afford after workup and purification using method-J. (S)-ethyl 3-(5-bromo-3-((2-(2-ethoxy-2-oxoethyl)-3-methylphenoxy)methyl)-1H-indazol-1-yl)pyrrolidine-1-carboxylate (343g) (710 mg, 83% yield) as a clear oil; MS (ES+): 544.1 (M+1).

### Step-6: Preparation of (S)-ethyl 3-(5-(1-aminoisoquinolin-7-yl)-3-((2-(2-ethoxy-2-oxoethyl)-3-methylphenoxy)methyl)-1H-indazol-1-yl)pyrrolidine-1-carboxylate (343h)

Compound 343h was prepared according to the procedure reported in step-5 of scheme-1, from (S)-ethyl 3-(5-bromo-3-((2-(2-ethoxy-2-oxoethyl)-3-methylphenoxy)methyl)-1H-indazol-1-yl)pyrrolidine-1-carboxylate (343g) (200 mg, 0.367 mmol) in dioxane/MeTHF (10 mL, ratio 9:1) using (1-aminoisoquinolin-7-yl)boronic acid (87a) (90 mg, 0.478 mmol). K₃PO₄ (4 M aqueous solution, 367 μl, 1.469 mmol). PCy₃ (20.60 mg, 0.073 mmol), Pd₂(dba)₃ (33.6 mg, 0.037 mmol) and PdCl₂(dppf)-CH₂Cl₂Adduct (30.0 mg, 0.037 mmol) to afford after workup and purification using method-AE, (S)-ethyl 3-(5-(1-aminoisoquinolin-7-yl)-3-((2-(2-ethoxy-2-oxoethyl)-3-methylphenoxy)methyl)-1H-indazol-1-yl)pyrrolidine-1-carboxylate (343h) (101 mg, 45% yield) as a yellow solid; MS (ES+): 608.3 (M+1).

### Step-7: Preparation of (S)-2-(2-((5-(1-aminoisoquinolin-7-yl)-1-(1-(ethoxycarbonyl)pyrrolidin-3-yl)-1H-indazol-3-yl)methoxy)-6-methylphenyl)acetic acid (343i)

Compound 343i was prepared according to the procedure reported in step-2 of scheme-1, from (S)-ethyl 3-(5-(1-aminoisoquinolin-7-yl)-3-((2-(2-ethoxy-2-oxoethyl)-3-methylphenoxy)methyl)-1H-indazol-1-yl)pyrrolidine-1-carboxylate (343h) (120 mg, 0.202 mmol) in THF (3 mL) using lithium hydroxide hydrate (25.4 mg, 0.606 mmol) in water (1 mL) and stirring overnight at RT to afford after work up and purification using method-M. (S)-2-(2-((5-(1-aminoisoquinolin-7-yl)-1-(1-(ethoxycarbonyl)pyrrolidin-3-yl)-1H-indazol-3-yl)methoxy)-6-methylphenyl)acetic acid (343i) (20.2 mg, 22% yield) HCl salt as a white solid; ¹H NMR (300 MHz, DMSO-d₆) δ 13.19 (s, 1H, D₂O Exchangeable), 12.10 (s, 1H, D₂O Exchangeable), 9.07 (s, 2H, D₂O Exchangeable), 8.94 (s, 1H), 8.38 (dd, J=8.5, 1.6 Hz, 1H), 8.31 (d, J=1.6 Hz, 1H), 8.08-8.00 (m, 2H), 7.99-7.90 (m, 1H), 7.69 (d, J=6.9 Hz, 1H), 7.27 (d, J=6.9 Hz, 1H), 7.17-7.08 (m, 2H), 6.85-6.77 (m, 1H), 5.65-5.49 (m, 1H), 5.46 (s, 2H), 4.07 (t, J=7.0 Hz, 2H), 3.97-3.81 (m, 1H), 3.75-3.63 (m, 2H), 3.58 (s, 2H), 3.57-3.48 (m, 1H), 2.47-2.31 (m, 2H), 2.19 (s, 3H), 1.29-1.12 (m, 3H); MS (ES+): 580.2 (M+1); (ES−): 578.2 (M−1); Optical Rotation: −3.636 [0.055. MeOH].

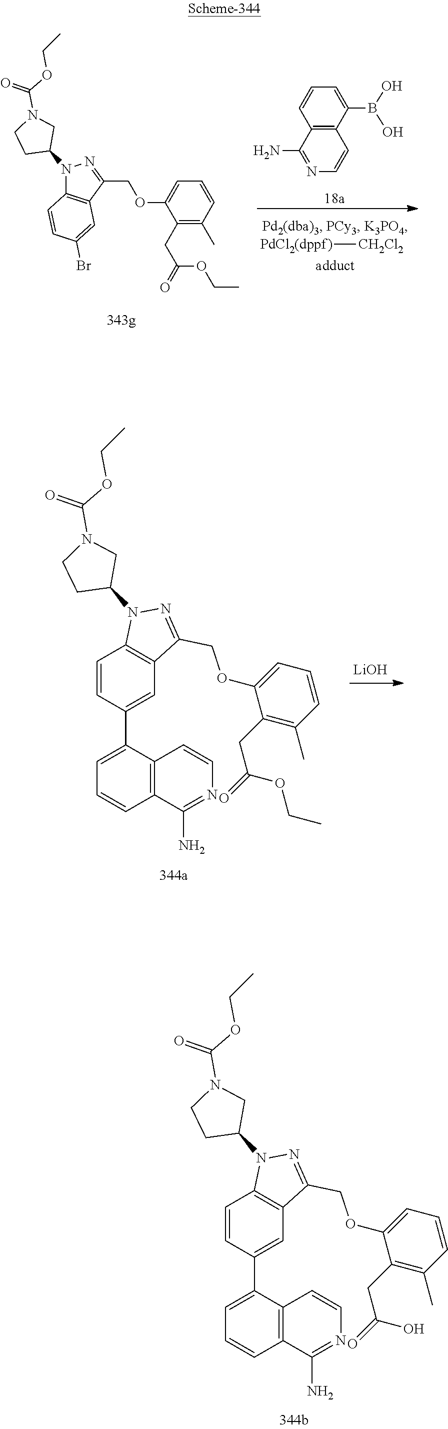

### Preparation of (S)-2-(2-((5-(1-aminoisoquinolin-5-yl)-1-(1-(ethoxycarbonyl)pyrrolidin-3-yl)-1H-indazol-3-yl)methoxy)-6-methylphenyl)acetic acid (344b)

### Step-1: Preparation of ethyl (S)-3-(5-(1-aminoisoquinolin-5-yl)-3-((2-(2-ethoxy-2-oxoethyl)-3-methylphenoxy)methyl)-1H-indazol-1-yl)pyrrolidine-1-carboxylate (344a)

Compound 344a was prepared according to the procedure reported in step-5 of scheme-1, from (S)-ethyl 3-(5-bromo-3-((2-(2-ethoxy-2-oxoethyl)-3-methylphenoxy)methyl)-1H-indazol-1-yl)pyrrolidine-1-carboxylate (343g) (200 mg, 0.367 mmol) in dioxane/MeTHF (10 mL, ratio 9:1) using (1-aminoisoquinolin-5-yl)boronic acid (18a) (90 mg, 0.478 mmol), K₃PO₄ (2 M aqueous solution, 735 al, 1.469 mmol), PCy₃ (21 mg, 0.073 mmol), Pd₂(dba)₃ (34 mg, 0.037 mmol) and PdCl₂(dppf)-CH₂Cl₂Adduct (30 mg, 0.037 mmol) to afford after workup and purification using method-AE, ethyl (S)-3-(5-(1-aminoisoquinolin-5-yl)-3-((2-(2-ethoxy-2-oxoethyl)-3-methylphenoxy)methyl)-1H-indazol-1-yl)pyrrolidine-1-carboxylate (344a) (98 mg, 44% yield) as a yellow solid; MS (ES+): 608.3 (M+1).

### Step-2: Preparation of (S)-2-(2-((5-(1-aminoisoquinolin-5-yl)-1-(1-(ethoxycarbonyl)pyrrolidin-3-yl)-1H-indazol-3-yl)methoxy)-6-methylphenyl)acetic acid (344b)

Compound 344b was prepared according to the procedure reported in step-2 of scheme-1, from ethyl (S)-3-(5-(1-aminoisoquinolin-5-yl)-3-((2-(2-ethoxy-2-oxoethyl)-3-methylphenoxy)methyl)-1H-indazol-1-yl)pyrrolidine-1-carboxylate (344a) (95 mg, 0.156 mmol) in THF (3 mL) using lithium hydroxide hydrate (19.68 mg, 0.469 mmol) in water (1 mL), stirring overnight at RT to afford after work up and purification using method-M, (S)-2-(2-((5-(1-aminoisoquinolin-5-yl)-1-(1-(ethoxycarbonyl)pyrrolidin-3-yl)-1H-indazol-3-yl)methoxy)-6-methylphenyl)acetic acid (344b) (21.3 mg, 24% yield) HCl salt as a white solid; ¹H NMR (300 MHz, DMSO-d₆) δ 13.11 (s, 1H, D₂O Exchangeable), 11.97 (s, 1H, D₂O Exchangeable), 8.99 (s, 2H, D₂O Exchangeable), 8.59 (d, J=8.3 Hz, 1H), 7.97-7.80 (m, 4H), 7.62 (d, J=7.2 Hz, 1H), 7.51 (dd, J=8.7, 1.6 Hz, 1H), 7.14-7.06 (m, 2H), 6.97 (d, J=7.2 Hz, 1H), 6.82-6.74 (m, 1H), 5.63-5.48 (m, 1H), 5.42 (s, 2H), 4.15-4.00 (m, 2H), 3.98-3.82 (m, 1H), 3.77-3.61 (m, 2H), 3.54 (s, 3H), 2.47-2.34 (m, 2H), 2.17 (s, 3H), 1.26-1.10 (m, 3H); MS (ES+): 580.3 (M+1); Optical Rotation: −8.00 [0.05, MeOH].

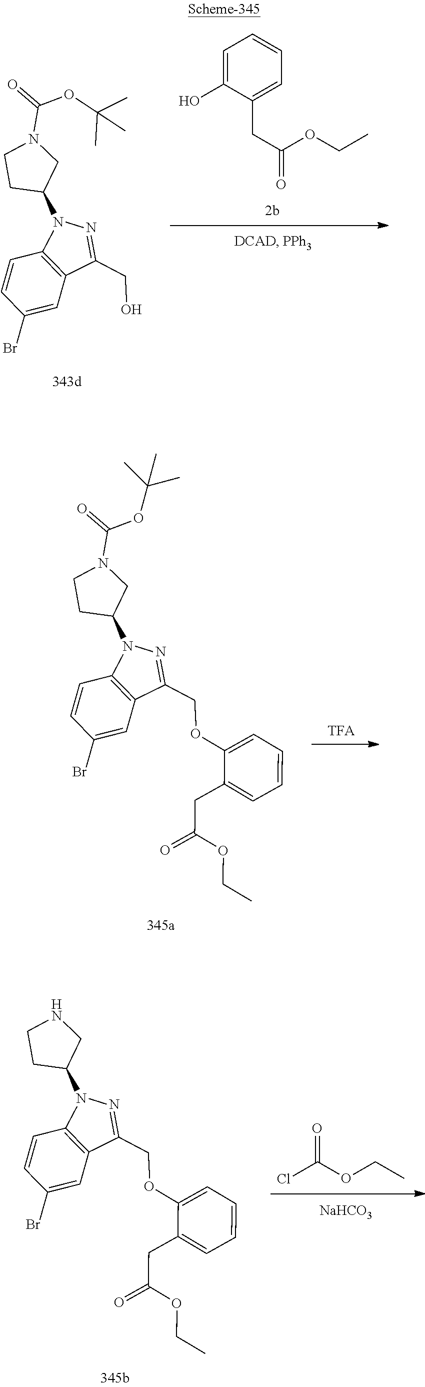

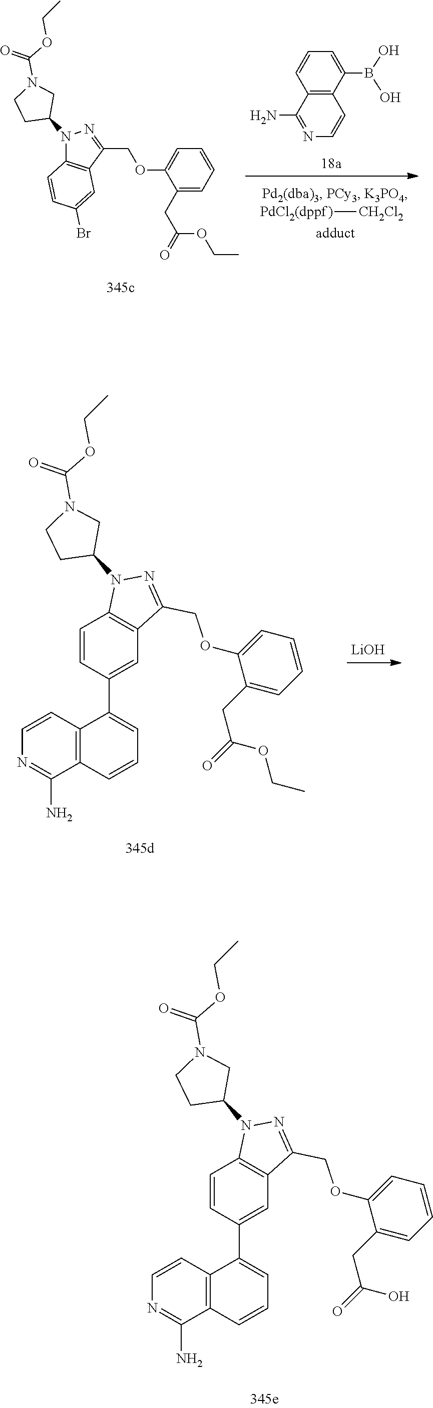

### Preparation of (S)-2-(2-((5-(1-aminoisoquinolin-5-yl)-1-(1-(ethoxycarbonyl)pyrrolidin-3-yl)-1H-indazol-3-yl)methoxy)phenyl)acetic acid (345e)

### Step-1: Preparation of (S)-tert-butyl 3-(5-bromo-3-((2-(2-ethoxy-2-oxoethyl)phenoxy)methyl)-1H-indazol-1-yl)pyrrolidine-1-carboxylate (345a)

Compound 345a was prepared according to the procedure reported in step-2 of scheme-2, from (S)-tert-butyl 3-(5-bromo-3-(hydroxymethyl)-1H-indazol-1-yl)pyrrolidine-1-carboxylate (343d) (1.5 g, 3.79 mmol) in DCM (25 mL) using ethyl 2-(2-hydroxyphenyl)acetate (2b) (0.819 g, 4.54 mmol), PPh₃ (1.092 g, 4.16 mmol) and a solution of DCAD (1.529 g, 4.16 mmol) in DCM (5 mL) to afford after workup and purification using method-BR, (S)-tert-butyl 3-(5-bromo-3-((2-(2-ethoxy-2-oxoethyl)phenoxy)methyl)-1H-indazol-1-yl)pyrrolidine-1-carboxylate (345a) (1.2 g, 57% yield) as a white solid; MS (ES+): 558.2 (M+1).

### Step-2: Preparation of (S)-ethyl 2-(2-((5-bromo-1-(pyrrolidin-3-yl)-1H-indazol-3-yl)methoxy)phenyl)acetate (345b)

Compound 345b was prepared according to the procedure reported in step-4 of scheme-9, from (S)-tert-butyl 3-(5-bromo-3-((2-(2-ethoxy-2-oxoethyl)phenoxy)methyl)-1H-indazol-1-yl)pyrrolidine-1-carboxylate (345a) (1.2 g, 2.153 mmol) in DCM (100 mL) using TFA (1.658 mL, 21.53 mmol) and stirring at RT for 16 h. The reaction mixture was concentrated and the residue was neutralized with sodium bicarbonate, extracted with ethyl acetate (2×50 mL), washed with water (100 mL), brine (50 mL) dried and concentrated in vacuum to afford (S)-ethyl 2-(2-((5-bromo-1-(pyrrolidin-3-yl)-1H-indazol-3-yl)methoxy)phenyl)acetate (345b) (947 mg, 96% yield) and was used as such without further purification; MS (ES+): 458.1 (M+1).

### Step-3: Preparation of (S)-ethyl 3-(5-bromo-3-((2-(2-ethoxy-2-oxoethyl)phenoxy)methyl)-1H-indazol-1-yl)pyrrolidine-1-carboxylate (345c)

Compound 345c was prepared according to the procedure reported in step-1 of scheme-126, from (S)-ethyl 2-(2-((5-bromo-1-(pyrrolidin-3-yl)-1H-indazol-3-yl)methoxy)phenyl)acetate (345b) (730 mg, 1.593 mmol) in THF (5 mL) and water (5 mL) using sodium bicarbonate (201 mg, 2.389 mmol) and ethyl carbonochloridate (216 μl, 2.389 mmol) to afford after workup and purification using method-J, (S)-ethyl 3-(5-bromo-3-((2-(2-ethoxy-2-oxoethyl)phenoxy)methyl)-1H-indazol-1-yl)pyrrolidine-1-carboxylate (345c) (743 mg, 88% yield) as a clear oil; MS (ES+): 530.1 (M+1).

### Step-4: Preparation of (S)-ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (345d)

Compound 345d was prepared according to the procedure reported in step-5 of scheme-1, from (S)-ethyl 3-(5-bromo-3-((2-(2-ethoxy-2-oxoethyl)phenoxy)methyl)-1H-indazol-1-yl)pyrrolidine-1-carboxylate (345c) (200 mg, 0.377 mmol) in dioxane/MeTHF (10 mL, ratio 9:1) using (1-aminoisoquinolin-5-yl)boronic acid (18a) (92 mg, 0.49 mmol), K₃PO₄ (2 M aqueous solution, 754 μl, 1.508 mmol), PCy₃ (21.15 mg, 0.075 mmol), Pd₂(dba)₃ (34.5 mg, 0.038 mmol) and PdCl₂(dppf)-CH₂Cl₂Adduct (30.8 mg, 0.038 mmol) to afford after workup and purification using method-AE, (S)-ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (345d) (114 mg, 51% yield) as a yellow solid; MS (ES+): 594.3 (M+1).

### Step-5: Preparation of (S)-2-(2-((5-(1-aminoisoquinolin-5-yl)-1-(1-(ethoxycarbonyl)pyrrolidin-3-yl)-1H-indazol-3-yl)methoxy)phenyl)acetic acid (345e)

Compound 345e was prepared according to the procedure reported in step-2 of scheme-1, from (S)-ethyl 3-(5-(1-aminoisoquinolin-5-yl)-3-((2-(2-ethoxy-2-oxoethyl)phenoxy)methyl)-1H-indazol-1-yl)pyrrolidine-1-carboxylate (345d) (110 mg, 0.185 mmol) in THF (3 mL) using lithium hydroxide hydrate (23.32 mg, 0.556 mmol) in water (1 mL) and stirring overnight at RT to afford after work up and purification using method-M. (S)-2-(2-((5-(1-aminoisoquinolin-5-yl)-1-(1-(ethoxycarbonyl)pyrrolidin-3-yl)-1H-indazol-3-yl)methoxy)phenyl)acetic acid (345e) (31.3 mg, 30% yield) HCl salt as a white solid; ¹H NMR (300 MHz, DMSO-d₆) δ 13.12 (s, 1H, D₂O Exchangeable), 11.99 (s, 1H, D₂O Exchangeable), 9.03 (s, 2H, D₂O Exchangeable), 8.60 (d, J=8.3 Hz, 1H), 7.98-7.78 (m, 4H), 7.62 (d, J=7.2 Hz, 1H), 7.51 (dd, J=8.6, 1.7 Hz, 1H), 7.27-7.21 (m, 2H), 7.19-7.14 (m, 1H), 6.98 (d, J=7.3 Hz, 1H), 6.93-6.86 (m, 1H), 5.61-5.51 (m, 1H), 5.45 (s, 2H), 4.13-3.98 (m, 2H), 3.94-3.83 (m, 1H), 3.76-3.60 (m, 2H), 3.60-3.50 (m, 1H), 3.47 (s, 2H), 2.48-2.34 (m, 2H), 1.31-1.07 (m, 3H); MS (ES+): 566.2 (M+1); (ES−): 564.2 (M−1); Optical Rotation: −14.118 [0.085, MeOH].

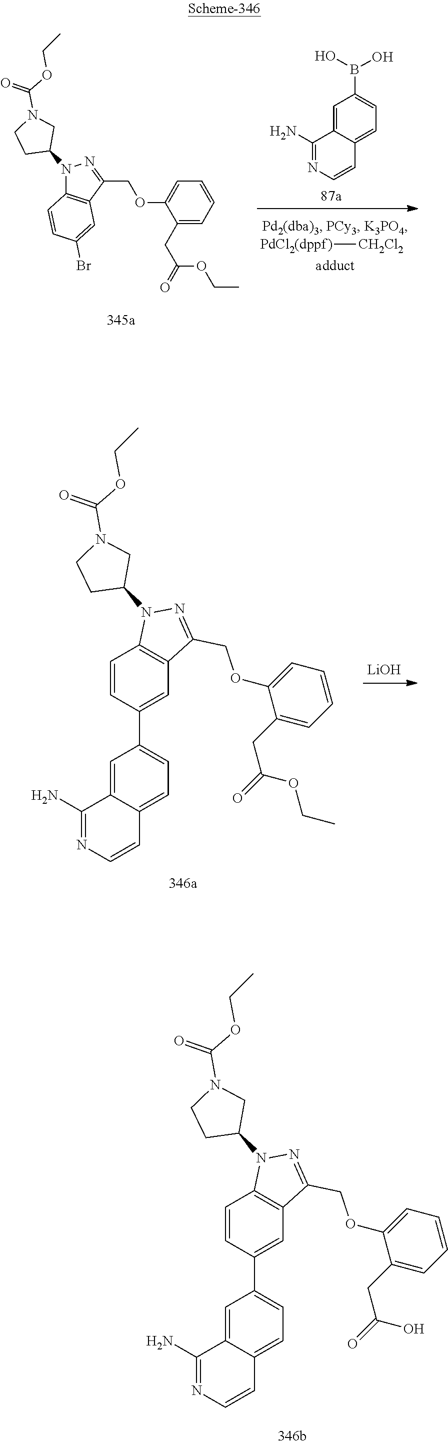

### Preparation of (S)-2-(2-((5-(1-aminoisoquinolin-7-yl)-1-(1-(ethoxycarbonyl)pyrrolidin-3-yl)-1H-indazol-3-yl)methoxy)phenyl)acetic acid (346b)

### Step-1: Preparation of (S)-ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (346a)

Compound 346a was prepared according to the procedure reported in step-5 of scheme-1, from (S)-ethyl 3-(5-bromo-3-((2-(2-ethoxy-2-oxoethyl)phenoxy)methyl)-1H-indazol-1-yl)pyrrolidine-1-carboxylate (345a) (200 mg, 0.377 mmol) in dioxane/MeTHF (10 mL, ratio 9:1) using (I-aminoisoquinolin-7-yl)boronic acid (87a) (92 mg, 0.49 mmol), K₃PO₄ (4M aqueous solution, 377 μl, 1.508 mmol), PCy₃ (21.15 mg, 0.075 mmol), Pd₂(dba)₃ (34.5 mg, 0.038 mmol) and PdCl₂(dppf)-CH₂Cl₂Adduct (30.8 mg, 0.038 mmol) to afford after workup and purification using method-AE, (S)-ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (346a) (121 mg, 54% yield) as a yellow solid; MS (ES+): 594.3 (M+1).

### Step-2: Preparation of (S)-2-(2-((5-(1-aminoisoquinolin-7-yl)-1-(1-(ethoxycarbonyl)pyrrolidin-3-yl)-1H-indazol-3-yl)methoxy)phenyl)acetic acid (346b)

Compound 346b was prepared according to the procedure reported in step-2 of scheme-1, from (S)-ethyl 3-(5-(1-aminoisoquinolin-7-yl)-3-((2-(2-ethoxy-2-oxoethyl)phenoxy)methyl)-1H-indazol-1-yl)pyrrolidine-1-carboxylate (346a) (120 mg, 0.202 mmol) in THF (3 mL) using lithium hydroxide hydrate (25.4 mg, 0.606 mmol) in water (1 mL) and stirring overnight at RT to afford after work up and purification using method-M, (S)-2-(2-((5-(1-aminoisoquinolin-7-yl)-1-(1-(ethoxycarbonyl)pyrrolidin-3-yl)-1H-indazol-3-yl)methoxy)phenyl)acetic acid (346b) (25.4 mg, 22% yield) HCl salt as a white solid; ¹H NMR (300 MHz, DMSO-d₆) δ 13.20 (s, 1H, D₂O Exchangeable), 12.15 (s, 1H, D₂O Exchangeable), 9.10 (s, 2H, D₂O Exchangeable), 8.95 (s, 1H), 8.42 (dd, J=8.7, 1.6 Hz, 1H), 8.33 (s, 1H), 8.09-8.00 (m, 2H), 7.99-7.91 (m, 1H), 7.69 (d, J=6.9 Hz, 1H), 7.34-7.24 (m, 3H), 7.24-7.15 (m, 1H), 6.98-6.87 (m, 1H), 5.63-5.52 (m, 1H), 5.49 (s, 2H), 4.07 (t, J=7.0 Hz, 2H), 3.97-3.84 (m, 1H), 3.75-3.61 (m, 2H), 3.60-3.46 (m, 3H), 2.47-2.29 (m, 2H), 1.27-1.15 (m, 3H); MS (ES+): 566.2 (M+1); (ES−): 564.2 (M−1); Optical Rotation: −7.41 [0.135, MeOH].

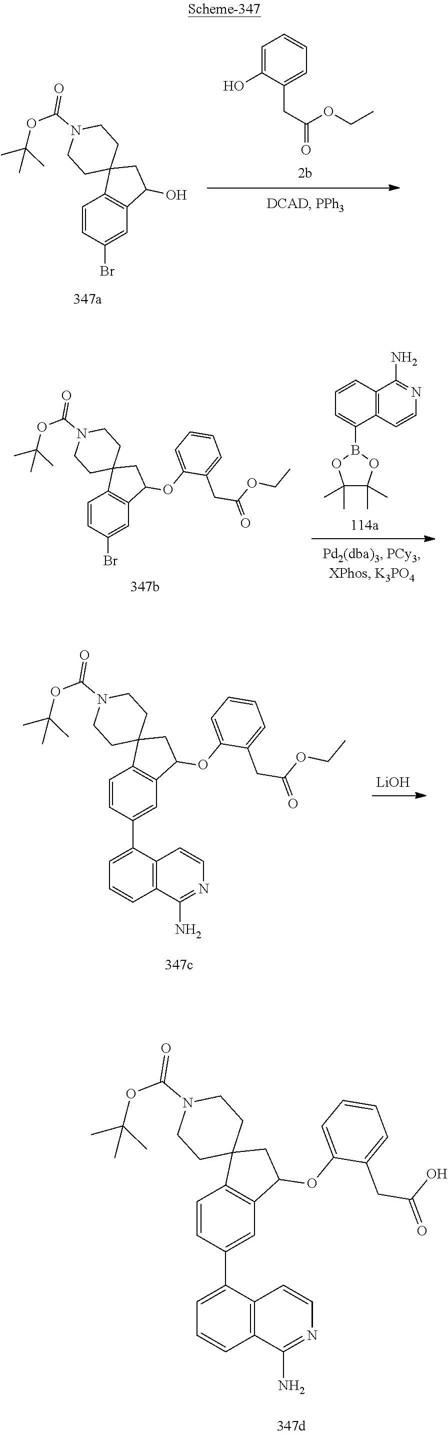

### Preparation of 2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (347d)

### Step-1: Preparation of tert-butyl 5-bromo-3-(2-(2-ethoxy-2-oxoethyl)phenoxy)-2,3-dihydrospiro[indene-1,4′-piperidine]-1′-carboxylate (347b)

Compound 347b was prepared according to the procedure reported in step-2 of scheme-2, from tert-butyl 5-bromo-3-hydroxy-2,3-dihydrospiro[indene-1,4′-piperidine]-1′-carboxylate (347a) (814 mg, 2.129 mmol; CAS #2660312-83-2) in DCM (100 mL) using PPh₃ (670 mg, 2.56 mmol), ethyl 2-(2-hydroxyphenyl)acetate (2b) (460 mg, 2.56 mmol) and a solution of DCAD (938 mg, 2.56 mmol) in DCM (60 mL) to afford after workup and purification using method-BS, tert-butyl 5-bromo-3-(2-(2-ethoxy-2-oxoethyl)phenoxy)-2,3-dihydrospiro[indene-1,4′-piperidine]-1′-carboxylate (347b) (540 mg, 47% yield) as a white solid; MS (ES+): 566.1 and 568.2 (M+Na)

### Step-2: Preparation of tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (347c)

Compound 347c was prepared according to the procedure reported in step-3 of scheme-112, from tert-butyl 5-bromo-3-(2-(2-ethoxy-2-oxoethyl)phenoxy)-2,3-dihydrospiro[indene-1,4′-piperidine]-1′-carboxylate (347b) (130 mg, 0.239 mmol) in dioxane/2-MeTHF (2 mL, 3:1) using 5-(4,4,5,5-tetramethyl-1,3,2-dioxaborolan-2-yl)isoquinolin-1-amine (114a) (129 mg, 0.478 mmol), K₃PO₄ (3M aqueous solution, 159 μl, 0.478 mmol), PCy₃ (26.8 mg, 0.096 mmol), XPhos (11.38 mg, 0.024 mmol), Pd₂(dba)₃ (43.7 mg, 0.048 mmol) and heating at 100° C. for 10 h to afford after workup and purification using method-U, tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (347c) (85 mg, 59% yield) as a clear oil; MS (ES+): 608.3 (M+1).

### Step-3: Preparation of 2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (347d)

Compound 347d was prepared according to the procedure reported in step-2 of scheme-1, from tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (347c) (85 mg, 0.140 mmol) in THF (3 mL) using lithium hydroxide (17.15 mg, 0.716 mmol) in water (1 mL) to afford after work up and purification using method-M, 2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (347d) (67 mg, 48% yield) HCl salt as a white solid; ¹H NMR (300 MHz, DMSO-d₆) δ 13.43 (s, 1H, D₂O exchangeable), 12.03 (s, 1H, D₂O exchangeable), 9.18 (s, 2H, D₂O exchangeable), 8.63 (d, J=8.2 Hz, 1H), 7.96-7.88 (m, 1H), 7.87-7.79 (m, 1H), 7.63 (d, J=7.2 Hz, 1H), 7.58-7.50 (m, 1H), 7.47-7.39 (m, 2H), 7.32-7.25 (m, 2H), 7.24-7.16 (m, 1H), 7.00-6.85 (m, 2H), 5.94 (dd, J=6.8, 4.5 Hz, 1H), 4.09-3.90 (m, 2H), 3.45 (d, J=1.8 Hz, 2H), 3.14-2.86 (m, 2H), 2.80 (dd, J=13.7, 6.9 Hz, 1H), 2.10 (dd, J=13.8, 4.5 Hz, 1H), 1.92 (td, J=12.8, 4.4 Hz, 1H), 1.85-1.72 (m, 1H), 1.72-1.63 (m, 1H), 1.64-1.53 (m, 1H), 1.45 (s, 9H); MS (ES+): 580.3 (M+1); (ES−): 578.2 (M−1).

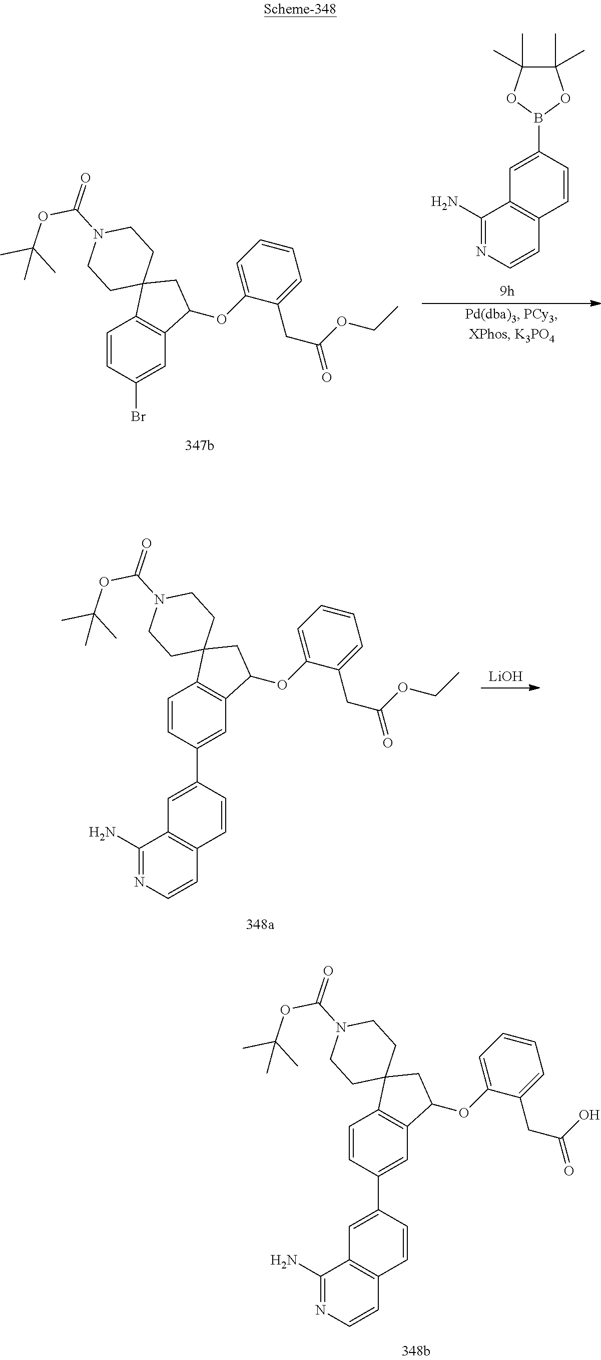

### Preparation of 2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (348b)

### Step-1: Preparation of tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (348a)

Compound 348a was prepared according to the procedure reported in step-3 of scheme-112, from tert-butyl 5-bromo-3-(2-(2-ethoxy-2-oxoethyl)phenoxy)-2,3-dihydrospiro[indene-1,4′-piperidine]-1′-carboxylate (347b) (130 mg, 0.239 mmol) in dioxane/2-MeTHF (2 mL, 3:1) using 7-(4,4,5,5-tetramethyl-1,3,2-dioxaborolan-2-yl)isoquinolin-1-amine (9h) (129 mg, 0.478 mmol), K₃PO₄ (3 M aqueous solution, 159 μl, 0.478 mmol), PCy₃ (27 mg, 0.096 mmol), XPhos (11.38 mg, 0.024 mmol). Pd₂(dba)₃ (44 mg, 0.048 mmol) and heating at 100° C. for 10 h to afford after workup and purification using method-U, tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (348a) (101 mg, 69.6% yield) as a clear oil; MS (ES+): 608.3 (M+1).

### Step-2: Preparation of 2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (348b)

Compound 348b was prepared according to the procedure reported in step-2 of scheme-1, from tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (348a) (101 mg, 0.166 mmol) in THF (3 mL) using lithium hydroxide (17.15 mg, 0.716 mmol) in water (1 mL) to afford after work up and purification using method-M, 2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (348b) (51 mg, 37% yield) HCl salt as a white solid; ¹H NMR (300 MHz, DMSO-d₆) δ 13.34 (s, 1H, D₂O exchangeable), 12.11 (s, 1H, D₂O exchangeable), 9.14 (s, 2H, D₂O exchangeable), 8.94-8.87 (m, 1H), 8.31 (dd, J=8.5, 1.6 Hz, 1H), 8.04 (d, J=8.5 Hz, 1H), 7.95-7.84 (m, 2H), 7.70 (d, J=6.9 Hz, 1H), 7.56 (d, J=8.0 Hz, 1H), 7.35-7.30 (m, 2H), 7.27 (d, J=6.9 Hz, 1H), 7.25-7.20 (m, 1H), 6.99-6.88 (m, 1H), 5.95 (dd, J=6.8, 4.2 Hz, 1H), 4.10-3.88 (m, 2H), 3.48 (d, J=2.9 Hz, 2H), 3.12-2.82 (m, 2H), 2.75 (dd, J=13.8, 6.9 Hz, 1H), 2.08 (dd, J=13.8, 4.3 Hz, 1H), 1.94-1.72 (m, 2H), 1.67-1.51 (m, 2H), 1.44 (s, 9H); MS (ES+): 580.3 (M+1); (ES−): 578.3 (M−1).

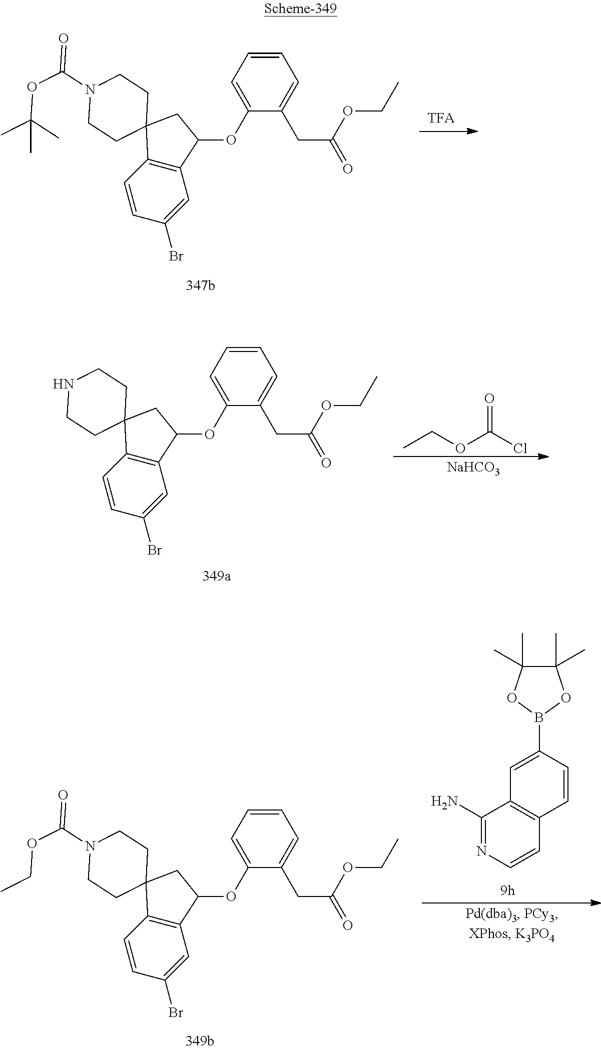

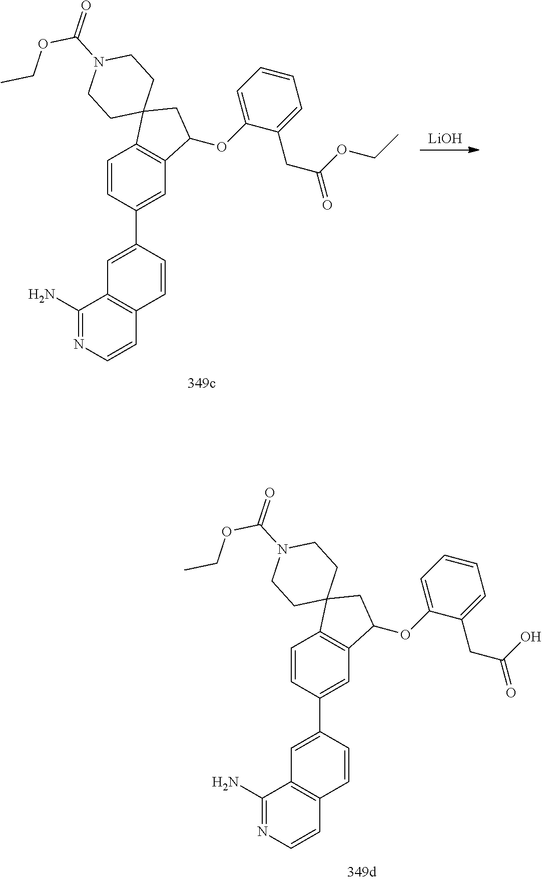

### Preparation of 2-(2-((5-(1-aminoisoquinolin-7-yl)-1′-(ethoxycarbonyl)-2,3-dihydrospiro[indene-1,4′-piperidin]-3-yl)oxy)phenyl)acetic acid (349d)

### Step-1: Preparation of ethyl 2-(2-((5-bromo-2,3-dihydrospiro[indene-1,4′-piperidin]-3-yl)oxy)phenyl)acetate (349a)

Compound 349a was prepared according to the procedure reported in step-4 of scheme-9, from tert-butyl 5-bromo-3-(2-(2-ethoxy-2-oxoethyl)phenoxy)-2,3-dihydrospiro[indene-1,4′-piperidine]-1′-carboxylate (347b) (350 mg, 0.643 mmol) in DCM (5 mL) using TFA (495 μl, 6.43 mmol) and stirring at RT for 2 h. The reaction mixture was concentrated to afford ethyl 2-(2-((5-bromo-2,3-dihydrospiro[indene-1,4′-piperidin]-3-yl)oxy)phenyl)acetate (349a) (270 mg, 95% yield) and was used as such without further purification; MS (ES+): 444.1 and 446.1 (M+1); (ES−): 442.0 and 444.1 (M−1).

### Step-2: Preparation of ethyl 5-bromo-3-(2-(2-ethoxy-2-oxoethyl)phenoxy)-2,3-dihydrospiro[indene-1,4′-piperidine]-1′-carboxylate (349b)

Compound 349b was prepared according to the procedure reported in step-1 of scheme-126, from ethyl 2-(2-((5-bromo-2,3-dihydrospiro[indene-1,4′-piperidin]-3-yl)oxy)phenyl)acetate (349a) (260 mg, 0.585 mmol) in THF (5 mL) and water (5 mL) using sodium bicarbonate (1463 μL, 2.93 mmol) and ethyl carbonochloridate (95 mg, 0.878 mmol) to afford after workup and purification using method-U, ethyl 5-bromo-3-(2-(2-ethoxy-2-oxoethyl)phenoxy)-2,3-dihydrospiro[indene-1,4′-piperidine]-1′-carboxylate (349b) (147 mg, 49% yield) as a clear oil; MS (ES+): 516.1 and $18.1 (M+1); 538.1 and 540.1 (M+Na).

### Step-3: Preparation of ethyl 5-(1-aminoisoquinolin-7-yl)-3-(2-(2-ethoxy-2-oxoethyl)phenoxy)-2,3-dihydrospiro[indene-1,4′-piperidine]-1′-carboxylate (349c)

Compound 349c was prepared according to the procedure reported in step-3 of scheme-112, from ethyl 5-bromo-3-(2-(2-ethoxy-2-oxoethyl)phenoxy)-2,3-dihydrospiro[indene-1,4′-piperidine]-1′-carboxylate (349b) (147 mg, 0.285 mmol) in dioxane/2-MeTHF (2 mL) using 7-(4,4,5,5-tetramethyl-1,3,2-dioxaborolan-2-yl)isoquinolin-1-amine (9h) (154 mg, 0.569 mmol), K₃PO₄ (3M aqueous solution, 190 μl, 0.569 mmol), PCy₃ (31.9 mg, 0.114 mmol), XPhos (13.57 mg, 0.028 mmol) and Pd₂(dba)₃ (52.1 mg, 0.057 mmol) and heating at 100° C. for 10 h to afford after workup and purification using method-M, ethyl 5-(1-aminoisoquinolin-7-yl)-3-(2-(2-ethoxy-2-oxoethyl)phenoxy)-2,3-dihydrospiro[indene-1,4′-piperidine]-1′-carboxylate (349c) (77 mg, 47% yield) as a white solid; ¹H NMR (300 MHZ, DMSO-d₆) δ 13.25 (s, 1H, D₂O exchangeable), 9.14 (s, 2H, D₂O exchangeable), 8.91 (s, 1H), 8.36-8.24 (m, 1H), 8.10-7.99 (m, 1H), 7.92 (dd, J=8.0, 1.8 Hz, 1H), 7.83 (d, J=1.7 Hz, 1H), 7.70 (d, J=6.9 Hz, 1H), 7.59 (d, J=8.1 Hz, 1H), 7.39-7.15 (m, 4H), 6.95 (ddd, J=7.8, 5.4, 3.0 Hz, 1H), 5.97 (d, J=4.4 Hz, 1H), 4.15-3.93 (m, 4H), 3.86-3.68 (m, 2H), 3.52 (s, 2H), 3.18-2.85 (m, 2H), 2.85-2.70 (m, 1H), 2.12-1.72 (m, 3H), 1.59 (t, J=12.6 Hz, 2H), 1.22 (t, J=7.0 Hz, 3H), 0.82 (t, J=7.1 Hz, 3H); MS (ES+): 580.3 (M+1); (ES−): 578.2 (M−1).

### Step-4: Preparation of 2-(2-((5-(1-aminoisoquinolin-7-yl)-1′-(ethoxycarbonyl)-2,3-dihydrospiro[indene-1,4′-piperidin]-3-yl)oxy)phenyl)acetic acid (349d)

Compound 349d was prepared according to the procedure reported in step-2 of scheme-1, from ethyl 5-(1-aminoisoquinolin-7-yl)-3-(2-(2-ethoxy-2-oxoethyl)phenoxy)-2,3-dihydrospiro[indene-1,4′-piperidine]-1′-carboxylate (349c) (50 mg, 0.086 mmol) in THF (3 mL) using lithium hydroxide (6.20 mg, 0.259 mmol) in water (1 mL) to afford after work up and purification using method-M, 2-(2-((5-(1-aminoisoquinolin-7-yl)-1′-(ethoxycarbonyl)-2,3-dihydrospiro[indene-1,4′-piperidin]-3-yl)oxy)phenyl)acetic acid (349d) (35 mg, 74% yield) HCl salt as a white solid; ¹H NMR (300 MHz, DMSO-d₆) δ 13.27 (s, 1H, D₂O exchangeable), 12.08 (s, 1H, D₂O exchangeable), 9.10 (s, 2H, D₂O exchangeable), 8.89 (d, J=1.7 Hz, 1H), 8.32 (dd, J=8.5, 1.6 Hz, 1H), 8.04 (d, J=8.5 Hz, 1H), 7.96-7.85 (m, 2H), 7.70 (d, J=6.9 Hz, 1H), 7.58 (d, J=8.0 Hz, 1H), 7.35-7.17 (m, 4H), 7.02-6.88 (m, 1H), 6.02-5.87 (m, 1H), 4.15-3.93 (m, 4H), 3.56-3.41 (m, 2H), 3.18-2.82 (m, 2H), 2.82-2.68 (m, 1H), 2.10 (dd, J=13.8, 4.1 Hz, 1H), 1.97-1.74 (m, 2H), 1.71-1.48 (m, 2H), 1.22 (t. J=7.1 Hz, 3H); MS (ES+): 552.3 (M+1); (ES−): 550.3 (M−1).

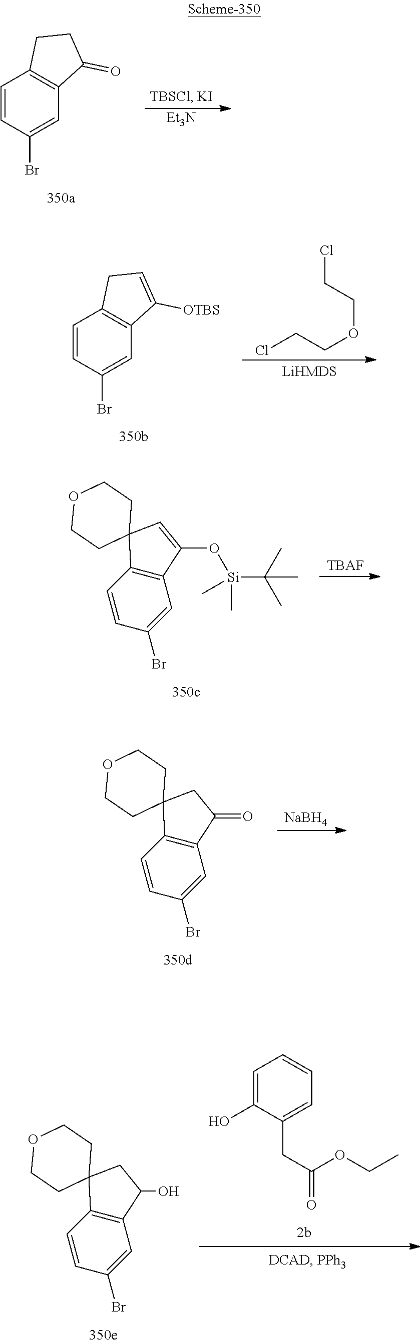

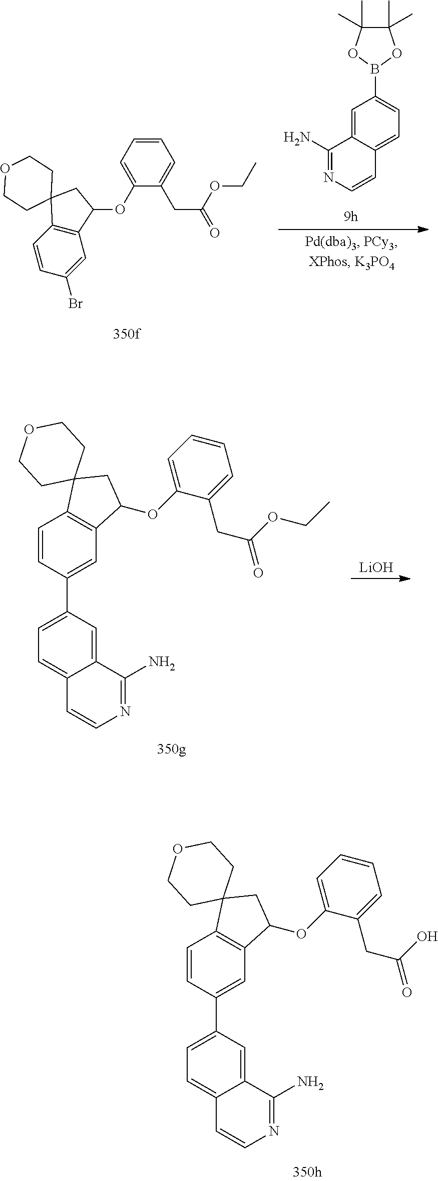 [SMILES: CCOC(=O)Cc1ccccc1OC1CC2(CCOCC2)c2ccc(Br)cc21 ; CC1(C)OB(c2ccc3ccnc(N)c3c2)OC1(C)C ; CCOC(=O)Cc1ccccc1OC1CC2(CCOCC2)c2ccc(-c3ccc4ccnc(N)c4c3)cc21 ; "LiOH" ; Nc1nccc2ccc(-c3ccc4c(c3)C(Oc3ccccc3CC(=O)O)CC43CCOCC3)cc12]

### Preparation of 2-(2-((5-(1-aminoisoquinolin-7-yl)-2,2′,3,3′,5′,6′-hexahydrospiro[indene-1,4′-pyran]-3-yl)oxy)phenyl)acetic acid (350h)

### Step-1: Preparation of ((5-bromo-1H-inden-3-yl)oxy)(tert-butyl)dimethylsilane (350b)

To a solution of 6-bromo-2,3-dihydro-1H-inden-1-one (10 g, 47.4 mmol; CAS #14548-39-1) in MeCN (100 mL) at RT was added triethylamine (6.60 mL, 47.4 mmol) followed by K1 (7.87 g, 47.4 mmol) and stirred for 10 min. To this mixture was added TBS-CI (7.14 g, 47.4 mmol) and heated at reflux for 4 h. The reaction mixture was cooled to RT and the solid was removed via filtration. The filtrate was purified by chromatography using method-A, to give ((5-bromo-1H-inden-3-yl)oxy)(tert-butyl)dimethylsilane (8.2 g, 53% yield) as a white solid; ME (ES+): 325.1 and 327.1 (M+1).

### Step-2: Preparation of ((5-bromo-2′,3′,5′,6′-tetrahydrospiro[indene-1,4′-pyran]-3-yl)oxy)(tert-butyl)dimethylsilane (350c)

To a solution of ((5-bromo-1H-inden-3-yl)oxy)(tert-butyl)dimethylsilane (350b) (4 g, 12.30 mmol) in anhydrous THF (25 mL) at −78° C. was added LiHMDS (1.0 M in THF) (30.7 mL, 30.7 mmol) and stirred at the same temperature for 30 min. A solution of 1-chloro-2-(2-chloroethoxy)ethane (2.11 g, 14.75 mmol) in anhydrous THF (2 mL) was added to the mixture allowed to warm to 0° C. and stirred at 0° C. for 8 h. The reaction was quenched with saturated NH₄Cl (20 mL) and extracted with EtOAc. The organic layer was separated washed with H₂O, brine, dried, filtered and concentrated in vacuo. The residue obtained was purified using method-J, to afford ((5-bromo-2′,3′,5′,6′-tetrahydrospiro[indene-1,4′-pyran]-3-yl)oxy)(tert-butyl)dimethylsilane (350c) (1.9 g, 39% yield) as a colorless oil; MS (ES+): 395.1 and 397.2 (M+1).

### Step-3: Preparation of 5-bromo-2′,3′,5′,6′-tetrahydrospiro[indene-1,4′-pyran]-3(2H)-one (350d)

To a solution of ((5-bromo-2′,3′,5′,6′-tetrahydrospiro[indene-1,4′-pyran]-3-yl)oxy)(tert-butyl)dimethylsilane (350c) (1.9 g, 4.81 mmol) in anhydrous THF (50 mL) was added TBAF (7.21 mL, 7.21 mmol) and stirred at RT for 30 min. The reaction mixture was quenched with saturated NH₄Cl (30 mL) and extracted with EtOAc (3×). The Combined organic layer was washed with H₂O, brine, dried, filtered and concentrated in vacuo. The residue obtained was purified using method-I, to afford 5-bromo-2′,3′,5′,6′-tetrahydrospiro[indene-1,4′-pyran]-3(2H)-one (350d) (1.01 g, 75% yield) as an off white solid; ¹H NMR (300 MHz, DMSO-d₆) δ 7.89 (dd, J=8.3, 1.9 Hz, 1H), 7.77-7.71 (m, 2H), 3.96-3.82 (m, 2H), 3.48 (td, J=12.2, 1.9 Hz, 2H), 2.76 (s, 2H), 2.03 (td, J=12.9, 4.7 Hz, 2H), 1.49-1.37 (m, 2H).

### Step-4: Preparation of 5-bromo-2.2′,3,3′5′,6′-hexahydrospiro[indene-1,4′-pyran]-3-ol (350e)

Compound 350e was prepared according to the procedure reported in step-1 of scheme-205, from 5-bromo-2′,3′5′,6′-tetrahydrospiro[indene-1,4′-pyran]-3(2H)-one (350d) (1 g, 3.56 mmol) in anhydrous MeOH (20 mL) using sodium borohydride (0.202 g, 5.34 mmol) to afford after work up and purification using method-1, 5-bromo-2,2′,3,3′,5′,6′-hexahydrospiro[indene-1,4′-pyran]-3-ol (350e) (1.01 g, >99% yield) as a yellow oil; ¹H NMR (300 MHz, DMSO-d₆) δ 7.49-7.39 (m, 2H), 7.25 (d, J=8.1 Hz, 1H), 5.46-5.31 (m, 1H), 5.09 (t, J=6.7 Hz, 1H), 3.89-3.77 (m, 2H), 3.65-3.42 (m, 2H), 2.61-2.53 (m, 1H), 2.08-1.91 (m, 1H), 1.83-1.63 (m, 2H), 1.50-1.39 (m, 1H), 1.33-1.24 (m, 1H).

### Step-5: Preparation of ethyl 2-(2-((5-bromo-2,2′3,3′,5′6′-hexahydrospiro[indene-1,4′-pyran]-3-yl)oxy)phenyl)acetate (350f)

Compound 350f was prepared according to the procedure reported in step-2 of scheme-2, from 5-bromo-2,2′,3,3′,5′,6′-hexahydrospiro[indene-1,4′-pyran]-3-ol (350e) (500 mg, 1.766 mmol) in DCM (20 mL) using PPh₃ (556 mg, 2.119 mmol), ethyl 2-(2-hydroxyphenyl)acetate (2b) (382 mg, 2.119 mmol) and a solution of DCAD (778 mg, 2.119 mmol) in DCM (10 mL) to afford after workup and purification using method-BS, ethyl 2-(2-((5-bromo-2,2′,3,3′,5′,6′-hexahydrospiro[indene-1,4′-pyran]-3-yl)oxy)phenyl)acetate (350f) (242.3 mg, 31% yield) as clear oil; MS (ES+): 445.1 and 447.1 (M+1); (ES−): 443.2 and 445.1 (M−1).

### Step-6: Preparation of ethyl 2-(2-((5-(1-aminoisoquinolin-7-yl)-2,2′,3,3′,5′,6′-hexahydrospiro[indene-1,4′-pyran]-3-yl)oxy)phenyl)acetate (350g)

Compound 350g was prepared according to the procedure reported in step-3 of scheme-112, from ethyl 2-(2-((5-bromo-2,2′,3,3′,5′,6′-hexahydrospiro[indene-1,4′-pyran]-3-yloxy)phenyl)acetate (350f) (120 mg, 0.269 mmol) in dioxane/2-MeTHF (2 mL) using 7-(4,4,5,5-tetramethyl-1,3,2-dioxaborolan-2-yl)isoquinolin-1-amine (9h) (146 mg, 0.539 mmol), K₃PO₄ (3 M aqueous solution, 180 μl, 0.539 mmol), PCy₃ (30.2 mg, 0.108 mmol), XPhos (25.7 mg, 0.054 mmol) and Pd₂(dba)₃ (49.3 mg, 0.054 mmol) and beating at 100° C. for 10 h to afford after workup and purification using method-U, ethyl 2-(2-((5-(1-aminoisoquinolin-7-yl)-2,2′,3,3′,5′,6′-hexahydrospiro[indene-1,4′-pyran]-3-yl)oxy)phenyl)acetate (350g) (45 mg, 33% yield) as a clear oil; MS (ES+): 509.3 (M+1).

### Step-7: Preparation of 2-(2-((5-(1-aminoisoquinolin-7-yl)-2,2′,3,3′,5′,6′-hexahydrospiro[indene-1,4′-pyran]-3-yl)oxy)phenyl)acetic acid (350h)

Compound 350h was prepared according to the procedure reported in step-2 of scheme-1, from ethyl 2-(2-((5-(1-aminoisoquinolin-7-yl)-2,2′,3,3′,5′,6′-hexahydrospiro[indene-1,4′-pyran]-3-yl)oxy)phenyl)acetate (350g) (45 mg, 0.088 mmol) in THF (3 mL) using lithium hydroxide (19.36 mg, 0.808 mmol) in water (1 mL) to afford after work up and purification using method-M, 2-(2-((5-(1-aminoisoquinolin-7-yl)-2,2′,3,3′,5′,6′-hexahydrospiro[indene-1,4′-pyran]-3-yl)oxy)phenyl)acetic acid (350h) (40 mg, 31% yield) HCl salt as a white solid; ¹H NMR (300 MHz, DMSO-d₆) δ 13.30 (s, 1H, D₂O exchangeable), 12.10 (s, 1H, D₂O exchangeable), 9.12 (s, 2H, D₂O) exchangeable), 8.90 (d, J=1.7 Hz, 1H), 8.31 (dd, J=8.4, 1.6 Hz, 1H), 8.03 (d, J=8.5 Hz, 1H), 7.93 (dd. J=8.0, 1.8 Hz, 1H), 7.90-7.83 (m, 1H), 7.70 (d, J=6.9 Hz, 1H), 7.58 (d, J=8.0 Hz, 1H), 7.31 (d, J=4.1 Hz, 2H), 7.29-7.18 (m, 2H), 7.00-6.88 (m, 1H), 5.99-5.89 (m, 1H), 3.98-3.79 (m, 2H), 3.68-3.56 (m, 1H), 3.54-3.42 (m, 3H), 2.86-2.74 (m, 1H), 2.18-1.87 (m, 3H), 1.62-1.39 (m, 2H); MS (ES+): 481.2 (M+1); (ES−): 479.3 (M−1).

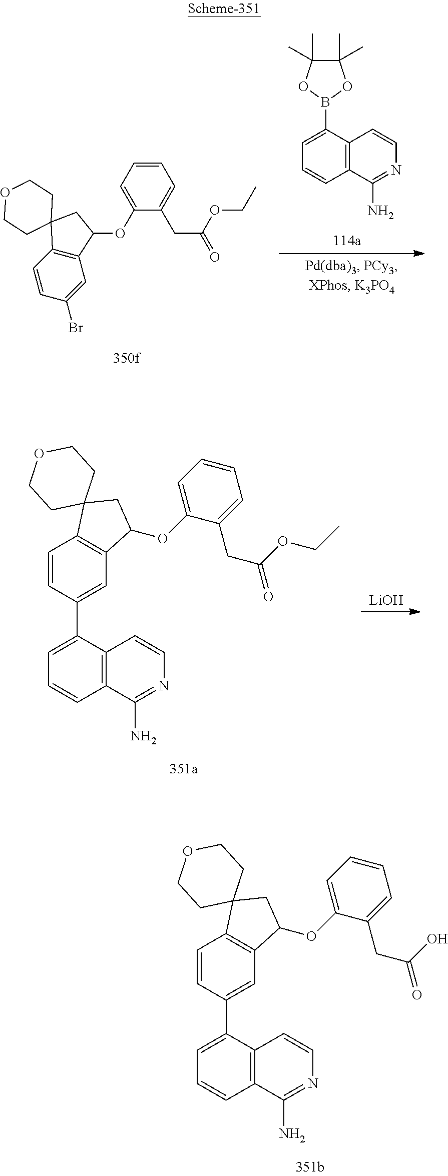 [SMILES: CCOC(=O)Cc1ccccc1OC1CC2(CCOCC2)c2ccc(Br)cc21 ; CC1(C)OB(c2cccc3c(N)nccc23)OC1(C)C ; CCOC(=O)Cc1ccccc1OC1CC2(CCOCC2)c2ccc(-c3cccc4c(N)nccc34)cc21 ; "LiOH" ; Nc1nccc2c(-c3ccc4c(c3)C(Oc3ccccc3CC(=O)O)CC43CCOCC3)cccc12]

### Preparation of 2-(2-((5-(1-aminoisoquinolin-5-yl)-2,2′,3,3′,5′,6′-hexahydrospiro[indene-1,4′-pyran]-3-yl)oxy)phenyl)acetic acid (351b)

### Step-1: Preparation of ethyl 2-(2-((5-(1-aminoisoquinolin-5-yl)-2,2′,3,3′,5′6′-hexahydrospiro[indene-1,4′-pyran]-3-yl)oxy)phenyl)acetate (351a)

Compound 351a was prepared according to the procedure reported in step-3 of scheme-112, from ethyl 2-(2-((5-bromo-2,2′,3,3′,5′,6′-hexahydrospiro[indene-1,4′-pyran]-3-yl)oxy)phenyl)acetate (350f) (120 mg, 0.269 mmol) in dioxane/2-MeTHF (2 mL) using 5-(4,4,5,5-tetramethyl-1,3,2-dioxaborolan-2-yl)isoquinolin-1-amine (114a) (146 mg, 0.539 mmol). K₃PO₄ (3 M aqueous solution, 180 μl, 0.539 mmol). PCy₃ (30.2 mg, 0.108 mmol). Xphos (25.7 mg, 0.054 mmol) and Pd₂(dba)₃ (49.3 mg, 0.054 mmol) and heating at 100° C. for 10 h to afford after workup and purification using method-U followed by method-M, ethyl 2-(2-((5-(1-aminoisoquinolin-5-yl)-2,2′,3,3′,5′,6′-hexahydrospiro[indene-1,4′-pyran]-3-yl)oxy)phenyl)acetate (351a) (91 mg, 66% yield) HCl salt as a white solid; ¹H NMR (300 MHz, DMSO-d₆) δ 13.24 (s, 1H, D₂O exchangeable), 9.15 (s, 2H, D₂O) exchangeable), 8.61 (d, J=8.2 Hz, 1H), 7.96-7.80 (m, 2H), 7.67-7.54 (m, 2H), 7.47 (dd, J=7.9, 1.7 Hz, 1H), 7.39-7.32 (m, 1H), 7.32-7.24 (m, 2H), 7.24-7.15 (m, 1H), 6.99 (d, J=7.3 Hz, 1H), 6.96-6.84 (m, 1H), 5.99-5.91 (m, 1H), 3.96-3.82 (m, 2H), 3.81-3.71 (m, 1H), 3.70-3.59 (m, 2H), 3.56-3.45 (m, 3H), 2.94-2.81 (m, 1H), 2.17-2.03 (m, 2H), 2.02-1.86 (m, 1H), 1.53 (t, J=12.1 Hz, 2H), 0.80 (t, J=7.1 Hz, 3H); MS (ES+): 509.2 (M+1).

### Step-2: Preparation of 2-(2-((5-(1-aminoisoquinolin-5-yl)-2,2′,3,3′,5′,6′-hexahydrospiro [indene-1,4′-pyran]-3-yl)oxy)phenyl)acetic acid (351b)

Compound 351b was prepared according to the procedure reported in step-2 of scheme-1, from ethyl 2-(2-((5-(1-aminoisoquinolin-5-yl)-2,2′,3,3′,5′,6′-hexahydrospiro[indene-1,4′-pyran]-3-yl)oxy)phenyl)acetate (351a) (91 mg, 0.179 mmol) in THF (3 mL) using lithium hydroxide (19.36 mg, 0.808 mmol) in water (1 mL) to afford after work up and purification using method-M, 2-(2-((5-(1-aminoisoquinolin-5-yl)-2,2′,3,3′,5′,6′-hexahydrospiro[indene-1,4′-pyran]-3-yl)oxy)phenyl)acetic acid (351b) (53 mg, 41% yield) HCl salt as a white solid; ¹H NMR (300 MHz, DMSO-d₆) δ 13.27 (s, 1H, D₂O exchangeable), 11.99 (s, 1H, D₂O exchangeable), 9.10 (s, 2H, D₂O exchangeable), 8.60 (d, J=8.2 Hz, 1H), 7.96-7.77 (m, 2H), 7.65-7.53 (m, 2H), 7.49-7.43 (m, 1H), 7.41 (d, J=1.7 Hz, 1H), 7.32-7.23 (m, 2H), 7.19 (d, J=7.3 Hz, 1H), 6.97 (d, J=7.2 Hz, 1H), 6.94-6.86 (m, 1H), 5.98-5.89 (m, 1H), 3.97-3.80 (m, 2H), 3.69-3.58 (m, 1H), 3.57-3.41 (m, 3H), 2.91-2.79 (m, 1H), 2.19-2.02 (m, 2H), 2.02-1.88 (m, 1H), 1.64-1.43 (m, 2H); MS (ES+): 481.2 (M+1); (ES−): 479.2 (M−1).

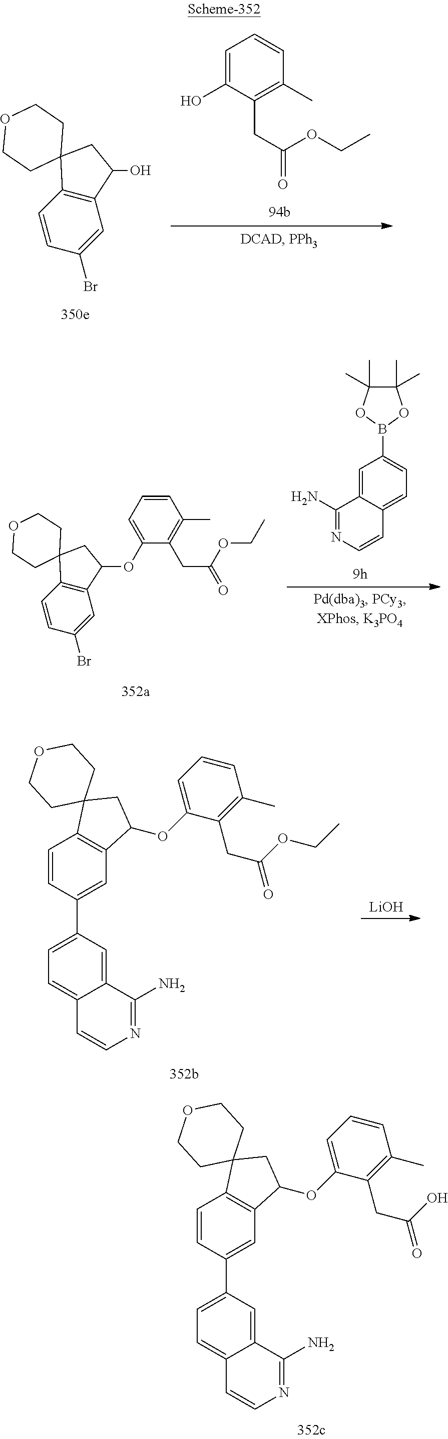

### Preparation of 2-(2-((5-(1-aminoisoquinolin-7-yl)-2,2′,3,3′,5′,6′-hexahydrospiro[indene-1,4′-pyran]-3-yl)oxy)-6-methylphenyl)acetic acid (352c)

### Step-1: Preparation of ethyl 2-(2-((5-bromo-2,2′,3,3′,5′,6′-hexahydrospiro[indene-1,4′-pyran]-3-yl)oxy)-6-methylphenyl)acetate (352a)

Compound 352a was prepared according to the procedure reported in step-2 of scheme-2, from 5-bromo-2,2′,3,3′,5′,6′-hexahydrospiro[indene-1,4′-pyran]-3-ol (350e) (500 mg, 1.766 mmol) in DCM (20 mL) using PPh₃ (556 mg, 2.119 mmol), ethyl 2-(2-hydroxy-6-methylphenyl)acetate (94b) (412 mg, 2.119 mmol) and a solution of DCAD (778 mg, 2.119 mmol) in DCM (60 mL) to afford after workup and purification using method-BS, ethyl 2-(2-((5-bromo-2,2′,3,3′,5′,6′-hexahydrospiro[indene-1,4′-pyran]-3-yl)oxy)-6-methylphenyl)acetate (352a) (161 mg, 20% yield) as clear oil; MS (ES+): 459.2 and 461.2 (M+1); 481.1 and 483.1 (M+Na).

### Step-2: Preparation of ethyl 2-(2-((5-(1-aminoisoquinolin-7-yl)-2,2′,3,3′,5′,6′-hexahydrospiro[indene-1,4′-pyran]-3-yl)oxy)-6-methylphenyl)acetate (352b)

Compound 352b was prepared according to the procedure reported in step-3 of scheme-112, from ethyl 2-(2-((5-bromo-2,2′,3,3′,5′,6′-hexahydrospiro[indene-1,4′-pyran]-3-yl)oxy)-6-methylphenyl)acetate (351a) (80 mg, 0.174 mmol) in dioxane/2-MeTHF (2 mL) using 7-(4,4,5,5-tetramethyl-1,3,2-dioxaborolan-2-yl)isoquinolin-1-amine (9h) (19.53 mg, 0.070 mmol), K₃PO₄ (3 M aqueous solution, 116 μl, 0.348 mmol). PCy₃ (19.53 mg, 0.070 mmol), XPhos (16.60 mg, 0.035 mmol), Pd₂(dba)₃ (31.9 mg, 0.035 mmol) and beating at 100° C. for 10 h to afford after workup and purification using method-U, ethyl 2-(2-((5-(1-aminoisoquinolin-7-yl)-2,2′,3,3′,5′,6′-hexahydrospiro[indene-1,4′-pyran]-3-yl)oxy)-6-methylphenyl)acetate (352b) (46 mg, 51% yield) as a clear oil; MS (ES+): 523.3 (M+1).

### Step-3: Preparation of 2-(2-((5-(1-aminoisoquinolin-7-yl)-2,2′,3,3′,5′,6′-hexahydrospiro[indene-1,4′-pyran]-3-yl)oxy)-6-methylphenyl)acetic acid (352c)

Compound 352c was prepared according to the procedure reported in step-2 of scheme-1, from ethyl 2-(2-((5-(1-aminoisoquinolin-7-yl)-2,2′,3,3′,5′,6′-hexahydrospiro[indene-1,4′-pyran]-3-yl)oxy)-6-methylphenyl)acetate (352b) (46 mg, 0.088 mmol) in THF (3 mL) using lithium hydroxide (12.51 mg, 0.522 mmol) in water (1 mL) to afford after work up and purification using method-M, 2-(2-((5-(1-aminoisoquinolin-7-yl)-2,2′,3,3′,5′,6′-hexahydrospiro[indene-1,4′-pyran]-3-yl)oxy)-6-methylphenyl)acetic acid (352c) (34 mg, 40% yield) HCl salt as a white solid; ¹H NMR (300 MHz, DMSO-d₆) δ 13.34 (s, 1H, D₂O exchangeable), 12.08 (s, 1H, D₂O exchangeable), 9.16 (s, 2H, DO exchangeable), 8.90 (d, J=1.7 Hz, 1H), 8.30 (dd, J=8.5, 1.6 Hz, 1H), 8.04 (d, J=8.5 Hz, 1H), 7.93 (dd, J=8.0, 1.8 Hz, 1H), 7.87 (d, J=1.7 Hz, 1H), 7.70 (d, J=6.9 Hz, 1H), 7.58 (d, J=8.0 Hz, 1H), 7.27 (d, J=7.0 Hz, 1H), 7.24-7.12 (m, 2H), 6.83 (dd, J=7.0, 1.7 Hz, 1H), 5.97-5.87 (m, 1H), 3.96-3.79 (m, 2H), 3.68-3.41 (m, 4H), 2.81-2.68 (m, 1H), 2.22 (s, 3H), 2.16-1.90 (m, 3H), 1.59-1.41 (m, 2H); MS (ES+): 495.2 (M+1); (ES−): 493.3 (M−1).

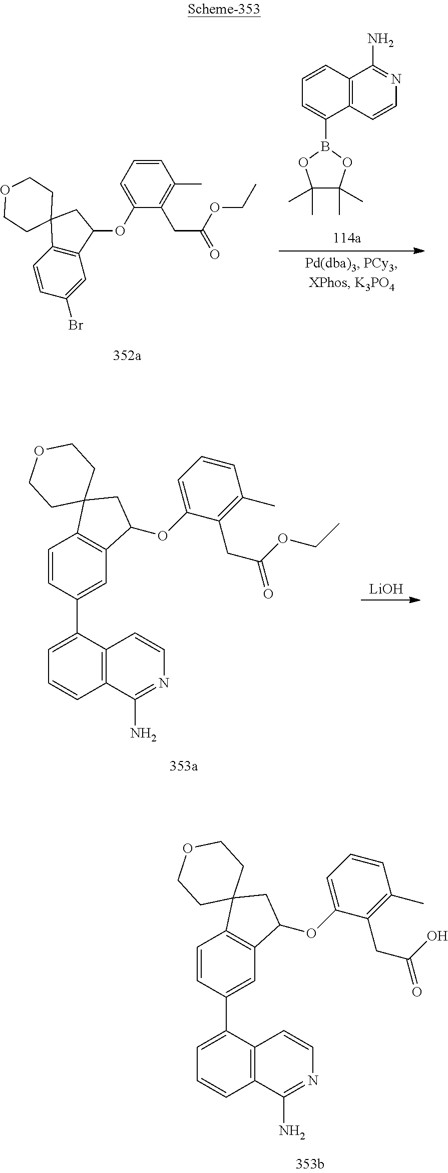

### Preparation of 2-(2-((5-(1-aminoisoquinolin-5-yl)-2,2′,3,3′,5′,6′-hexahydrospiro[indene-1,4′-pyran]-3-yl)oxy)-6-methylphenyl)acetic acid (353b)

### Step-1: Preparation of ethyl 2-(2-((5-(1-aminoisoquinolin-5-yl)-2,2′,3,3′,5′,6′-hexahydrospiro[indene-1,4′-pyran]-3-yl)oxy)-6-methylphenyl)acetate (353a)

Compound 353a was prepared according to the procedure reported in step-3 of scheme-112, from ethyl 2-(2-((5-bromo-2,2′,3,3′,5′,6′-hexahydrospiro[indene-1,4′-pyran]-3-yl)oxy)-6-methylphenyl)acetate (352a) (80 mg, 0.174 mmol) in dioxane/2-MeTHF (2 mL) using 5-(4,4,5,5-tetramethyl-1,3,2-dioxaborolan-2-yl)isoquinolin-1-amine (114a) (94 mg, 0.348 mmol). K₃PO₄ (3M aqueous solution, 116 μl, 0.348 mmol), PCy₃ (19.53 mg, 0.070 mmol), Xphos (16.60 mg, 0.035 mmol), Pd₂(dba)₃ (31.9 mg, 0.035 mmol) and heating at 100° C. for 10 h to afford after workup and purification using method-U, ethyl 2-(2-((5-(1-aminoisoquinolin-5-yl)-2,2′,3,3′,5′,6′-hexahydrospiro[indene-1,4′-pyran]-3-yl)oxy)-6-methylphenyl)acetate (353a) (61 mg, 67% yield) as a clear oil; ¹H NMR (300 MHz, DMSO-d₆) δ 13.14 (s, 1H, DO) exchangeable), 9.07 (s, 2H, D₂O exchangeable), 8.54 (d, J=8.2 Hz, 1H), 7.88-7.71 (m, 2H), 7.59-7.46 (m, 2H), 7.40 (dd. J=7.9, 1.7 Hz, 1H), 7.27 (d, J=1.6 Hz, 1H), 7.16-7.03 (m, 2H), 6.92 (d, J=7.2 Hz, 1H), 6.79-6.70 (m, 1H), 5.91-5.79 (m, 1H), 3.91-3.76 (m, 2H), 3.76-3.51 (m, 3H), 3.50-3.37 (m, 3H), 2.84-2.71 (m, 1H), 2.13 (s, 3H), 2.10-1.96 (m, 2H), 1.97-1.81 (m, 1H), 1.46 (t, J=13.1 Hz, 2H), 0.76 (t, J=7.1 Hz, 3H); MS (ES+): 523.3 (M+1).

### Step-2: Preparation of 2-(2-((5-(1-aminoisoquinolin-5-yl)-2,2′,3,3′,5′,6′-hexahydrospiro[indene-1,4′-pyran]-3-yl)oxy)-6-methylphenyl)acetic acid (353b)

Compound 353b was prepared according to the procedure reported in step-2 of scheme-1, from ethyl 2-(2-((5-(1-aminoisoquinolin-5-yl)-2,2′,3,3′5′,6′-hexahydrospiro[indene-1,4′-pyran]-3-yl)oxy)-6-methylphenyl)acetate (353a) (61 mg, 0.117 mmol) in THF (3 mL) using lithium hydroxide (12.51 mg, 0.522 mmol) in water (1 mL) to afford after work up and purification using method-M, 2-(2-((5-(1-aminoisoquinolin-5-yl)-2,2′,3,3′5′,6′-hexahydrospiro[indene-1,4′-pyran]-3-yl)oxy)-6-methylphenyl)acetic acid (353b) (30 mg, 35% yield) HCl salt as a white solid; ¹H NMR (300 MHz, DMSO-d₆) δ 13.37 (s, 1H, D₂O exchangeable), 12.01 (s, 1H, D₂O exchangeable), 9.13 (s, 2H, D₂O exchangeable), 8.61 (d, J=8.2 Hz, 1H), 7.95-7.87 (m, 1H), 7.87-7.78 (m, 1H), 7.66-7.53 (m, 2H), 7.46 (dd, J=7.8, 1.7 Hz, 1H), 7.40 (d, J=1.6 Hz, 1H), 7.22-7.06 (m, 2H), 6.97 (d, J=7.2 Hz, 1H), 6.84-6.76 (m, 1H), 5.96-5.82 (m, 1H), 3.97-3.81 (m, 2H), 3.69-3.58 (m, 1H), 3.57-3.43 (m, 3H), 2.87-2.76 (m, 1H), 2.20 (s, 3H), 2.18-1.89 (m, 3H), 1.63-1.44 (m, 2H); MS (ES+): 495.2 (M+1); (ES−): 493.2 (M−1).

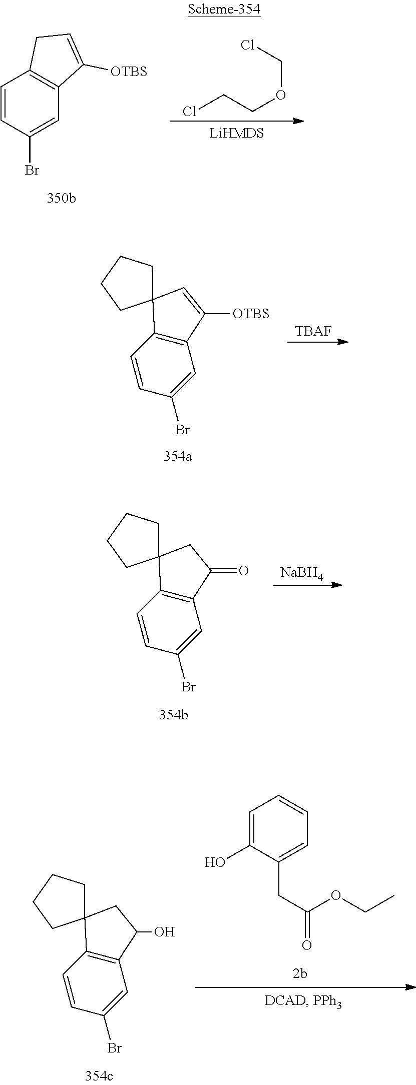

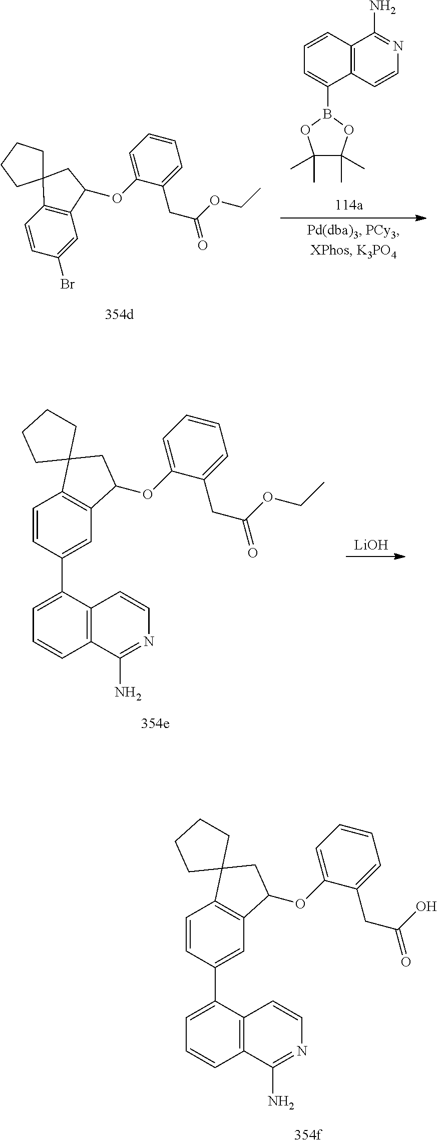

### Preparation of 2-(2-((5′-(1-aminoisoquinolin-5-yl)-2′,3′-dihydrospiro[cyclopentane-1,1′-inden]-3′-yl)oxy)phenyl)acetic acid (354f)

### Step-1: Preparation of ((5′-bromospiro[cyclopentane-1,1′-inden]-3′-yl)oxy)(tert-butyl)dimethylsilane (354a)

Compound 354a was prepared according to the procedure reported in step-2 of scheme-350, from ((5-bromo-1H-inden-3-yl)oxy)(ter-butyl)dimethylsilane (350b) (4 g, 12.30 mmol) in anhydrous THF (30 mL) using LiHMDS (1.0 M in THF) (30.7 mL, 30.7 mmol), a solution of 1,4-dichlorobutane (1.874 g, 14.75 mmol) in anhydrous THF (2 mL) to afford after work up and purification using method-I, ((5′-bromospiro[cyclopentane-1,1′-inden]-3′-yl)oxy)(tert-butyl)dimethylsilane (354a) (1.5 g, 3.95 mmol) as a colorless oil; ¹H NMR (300 MHz, DMSO-d₆) δ 7.17-7.06 (m, 2H), 6.99 (d, J=1.7 Hz, 1H), 5.36 (s, 1H), 1.76-1.56 (m, 6H), 1.52-1.39 (m, 2H), 0.74 (s, 9H), 0.00 (s, 6H).

### Step-2: Preparation of 5′-bromospiro[cyclopentane-1,1′-inden]-3′(2′H)-one (354b)

Compound 354b was prepared according to the procedure reported in step-3 of scheme-350, from ((5′-bromospiro[cyclopentane-1,1′-inden]-3′-yl)oxy)(ter-butyl)dimethylsilane (354a) (1.5 g, 3.95 mmol) in anhydrous THF (50 mL) using TBAF (5.93 mL, 5.93 mmol) to afford after work up and purification using method-I, 5′-bromospiro[cyclopentane-1,1′-inden]-3′(2′H)-one (354b) (930 mg, 3.51 mmol) as a yellow oil; ¹H NMR (300 MHz, DMSO-d₆) δ 7.84 (dd, J=8.2, 2.0 Hz, 1H), 7.68 (d, J=2.0 Hz, 1H), 7.64 (d, J=8.3 Hz, 1H), 2.57 (s, 2H), 1.93-1.80 (m, 4H), 1.80-1.64 (m, 4H); MS (ES+): 265.0 and 267.0 (M+1).

### Step-3: Preparation of 5′-bromo-2′,3′-dihydrospiro[cyclopentane-1,1′-inden]-3′-ol (354c)

Compound 354c was prepared according to the procedure reported in step-1 of scheme-205, from 5′-bromospiro[cyclopentane-1,1′-inden]-3′(2′H)-one (354b) (2.4 g, 9.05 mmol) in anhydrous MeOH (20 mL) using sodium borohydride (0.514 g, 13.58 mmol) to afford after work up and purification using method-J, 5′-bromo-2′,3′-dihydrospiro[cyclopentane-1,1′-inden]-3′-ol (354c) (2.3 g, 95% yield) as a white solid; ¹H NMR (300 MHz, DMSO-d₆) δ 7.44-7.35 (m, 2H), 7.15 (d, J=8.0 Hz, 1H), 5.45-5.17 (m, 1H), 5.04 (t, J=7.0 Hz, 1H), 2.33-2.22 (m, 1H), 1.94-1.61 (m, 8H), 1.55-1.42 (m, 1H).

### Step-4: Preparation of ethyl 2-(2-((5′-bromo-2′,3′-dihydrospiro[cyclopentane-1,1′-inden]-3′-yl)oxy)phenyl)acetate (354d)

Compound 354d was prepared according to the procedure reported in step-2 of scheme-2, from 5′-bromo-2′,3′-dihydrospiro[cyclopentane-1,1′-inden]-3′-ol (354c) (1.1 g, 4.12 mmol) in DCM (20 mL) using PPh₃ (1.296 g, 4.94 mmol), ethyl 2-(2-hydroxyphenyl)acetate (2b) (0.890 g, 4.94 mmol) and a solution of DCAD (1.814 g, 4.94 mmol) in DCM (60 mL) to afford after workup and purification using method-BS, ethyl 2-(2-((5′-bromo-2′,3′-dihydrospiro[cyclopentane-1,1′-inden]-3′-yl)oxy)phenyl)acetate (354d) (1.1 g, 62% yield) as clear oil; MS (ES+): 429.3 and 431.1 (M+1).

### Step-5: Preparation of ethyl 2-(2-((5′-(1-aminoisoquinolin-5-yl)-2′,3′-dihydrospiro[cyclopentane-1,1′-inden]-3′-yl)oxy)phenyl)acetate (354e)

Compound 354e was prepared according to the procedure reported in step-3 of scheme-112, from ethyl 2-(2-((5′-bromo-2′3′-dihydrospiro[cyclopentane-1,1′-inden]-3′-yl)oxy)phenyl)acetate (354d) (200 mg, 0.466 mmol) in dioxane/2-MeTHF (2 mL) using 5-(4,4,5,5-tetramethyl-1,3,2-dioxaborolan-2-yl)isoquinolin-1-amine (114a) (252 mg, 0.932 mmol), K₃PO₄ (3 M aqueous solution, 311 μl, 0.932 mmol), PCy₃ (52.3 mg, 0.186 mmol). XPhos (44.4 mg, 0.093 mmol). Pd₂(dba)₃ (85 mg, 0.093 mmol) and heating at 100° C. for 10 h to afford after workup and purification using method-U, ethyl 2-(2-((5′-(1-aminoisoquinolin-5-yl)-2′,3′-dihydrospiro[cyclopentane-1,1′-inden]-3′-yl)oxy)phenyl)acetate (354e) (200 mg, 87% yield) as a clear oil; MS (ES+): 493.2 (M+1).

### Step-6: Preparation of 2-(2-((5′-(1-aminoisoquinolin-5-yl)-2′,3′-dihydrospiro[cyclopentane-1,1′-inden]-3′-yl)oxy)phenyl)acetic acid (354f)

Compound 354f was prepared according to the procedure reported in step-2 of scheme-1, from ethyl 2-(2-((5′-(1-aminoisoquinolin-5-yl)-2′,3′-dihydrospiro[cyclopentane-1,1′-inden]-3′-yl)oxy)phenyl)acetate (354e) (200 mg, 0.406 mmol) in THF (3 mL) using lithium hydroxide (33.5 mg, 1.397 mmol) in water (1 mL) to afford after work up and purification using method-M, 2-(2-((5′-(1-aminoisoquinolin-5-yl)-2′,3′-dihydrospiro[cyclopentane-1,1′-inden]-3′-yl)oxy)phenyl)acetic acid (354f) (30 mg, 14% yield) HCl salt as a white solid; ¹H NMR (300 MHz, DMSO-d₆) & 13.22 (s, 1H, D₂O exchangeable), 11.99 (s, 1H, D₂O exchangeable), 9.08 (s, 2H, D₂O exchangeable), 8.59 (d, J=8.2 Hz, 1H), 7.91 (dd, J=7.4, 1.2 Hz, 1H), 7.83 (t, J=7.8 Hz, 1H), 7.61 (d, J=7.3 Hz, 1H), 7.49 (d, J=7.9 Hz, 1H), 7.46-7.40 (m, 1H), 7.37 (d, J=1.7 Hz, 1H), 7.30-7.15 (m, 3H), 6.98 (d, J=7.3 Hz, 1H), 6.94-6.82 (m, 1H), 5.89 (t, J=5.9 Hz, 1H), 3.45 (s, 2H), 2.71-2.59 (m, 1H), 2.07-1.94 (m, 2H), 1.93-1.66 (m, 7H); MS (ES+): 465.2 (M+1); (ES): 463.1 (M−1).

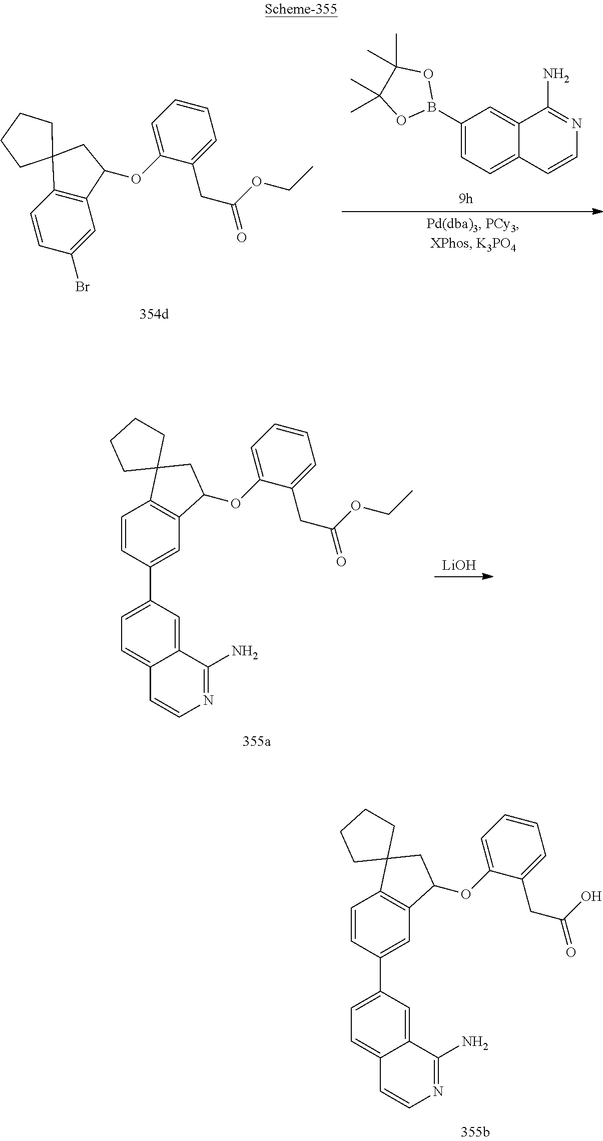 [SMILES: CCOC(=O)Cc1ccccc1OC1CC2(CCCC2)c2ccc(Br)cc21 ; CC1(C)OB(c2ccc3ccnc(N)c3c2)OC1(C)C ; CCOC(=O)Cc1ccccc1OC1CC2(CCCC2)c2ccc(-c3ccc4ccnc(N)c4c3)cc21 ; "LiOH" ; Nc1nccc2ccc(-c3ccc4c(c3)C(Oc3ccccc3CC(=O)O)CC43CCCC3)cc12]

### Preparation of 2-(2-((5′-(1-aminoisoquinolin-7-yl)-2′,3′-dihydrospiro[cyclopentane-1,1′-inden]-3′-yl)oxy)phenyl)acetic acid (355b)

### Step-1: Preparation of ethyl 2-(2-((5′-(1-aminoisoquinolin-7-yl)-2′3′-dihydrospiro[cyclopentane-1,1′-inden]-3′-yl)oxy)phenyl)acetate (355a)

Compound 355a was prepared according to the procedure reported in step-3 of scheme-112, from ethyl 2-(2-((5′-bromo-2′,3′-dihydrospiro[cyclopentane-1,1′-inden]-3′-yl)oxy)phenyl)acetate (354d) (200 mg, 0.466 mmol) in dioxane/2-MeTHF (2 mL) using 7-(4,4,5,5-tetramethyl-1,3,2-dioxaborolan-2-yl)isoquinolin-1-amine (9h) (252 mg, 0.932 mmol), K₃PO₄ (3M aqueous solution, 311 μl, 0.932 mmol), PCy₃ (52.3 mg, 0.186 mmol), XPhos (44.4 mg, 0.093 mmol). Pd₂(dba)₃ (85 mg, 0.093 mmol) and heating at 100° C. for 10 h to afford after workup and purification using method-U, ethyl 2-(2-((5′-(1-aminoisoquinolin-7-yl)-2′,3′-dihydrospiro[cyclopentane-1,1′-inden]-3′-yl)oxy)phenyl)acetate (355a) (70 mg, 31% yield) as a clear oil; MS (ES+): 493.2 (M+1).

### Step-2: Preparation of 2-(2-((5′-(1-aminoisoquinolin-7-yl)-2′,3′-dihydrospiro[cyclopentane-1,1′-inden]-3′-yl)oxy)phenyl)acetic acid (355b)

Compound 355b was prepared according to the procedure reported in step-2 of scheme-1, from ethyl 2-(2-((5′-(1-aminoisoquinolin-7-yl)-2′,3′-dihydrospiro[cyclopentane-1,1′-inden]-3′-yl)oxy)phenyl)acetate (355a) (70 mg, 0.142 mmol) in THF (3 mL) using lithium hydroxide (33.5 mg, 1.397 mmol) in water (1 mL) to afford after work up and purification using method-M, 2-(2-((5′-(1-aminoisoquinolin-7-yl)-2′,3′-dihydrospiro[cyclopentane-1,1′-inden]-3′-yl)oxy)phenyl)acetic acid (355b) (45 mg, 21% yield) HCl salt as a white solid; ¹H NMR (300 MHz, DMSO-d₆) δ 13.30 (s, 1H, D₂O exchangeable), 12.10 (s, 1H, D₂O exchangeable), 9.13 (s, 2H, D₂O exchangeable), 8.89 (d, J=1.7 Hz, 1H), 8.30 (dd, J=8.5, 1.6 Hz, 1H), 8.03 (d, J=8.5 Hz, 1H), 7.90 (dd, J=8.0, 1.8 Hz, 1H), 7.84 (d, J=1.7 Hz, 1H), 7.69 (d, J=6.9 Hz, 1H), 7.49 (d, J=8.0 Hz, 1H), 7.33-7.18 (m, 4H), 6.97-6.86 (m, 1H), 5.95-5.82 (m, 1H), 3.49 (d, J=3.1 Hz, 2H), 2.67-2.56 (m, 1H), 2.03-1.91 (m, 2H), 1.91-1.67 (m, 7H); MS (ES+): 465.2 (M+1); (ES−): 463.3 (M−1).

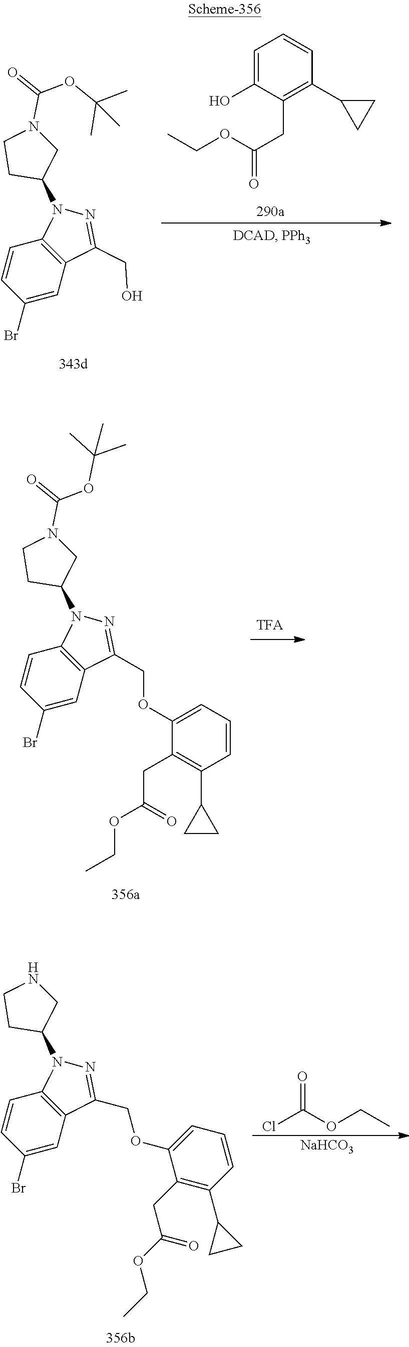

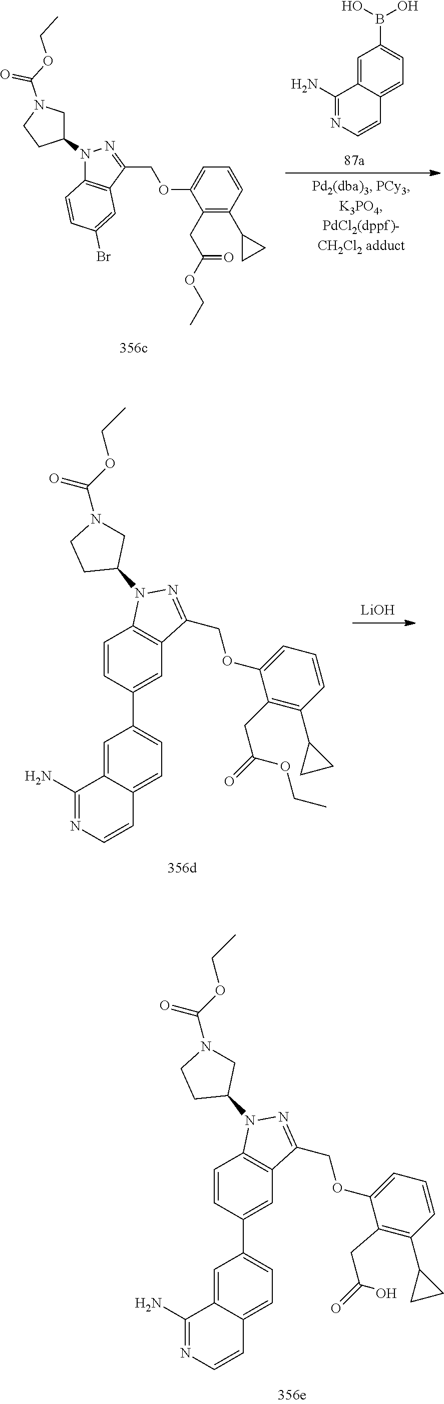 [SMILES: CCOC(=O)Cc1c(OCc2nn([C@H]3CCN(C(=O)OCC)C3)c3ccc(Br)cc23)cccc1C1CC1 ; Nc1nccc2ccc(B(O)O)cc12 ; CCOC(=O)Cc1c(OCc2nn([C@H]3CCN(C(=O)OCC)C3)c3ccc(-c4ccc5ccnc(N)c5c4)cc23)cccc1C1CC1 ; "LiOH" ; CCOC(=O)N1CC[C@H](n2nc(COc3cccc(C4CC4)c3CC(=O)O)c3cc(-c4ccc5ccnc(N)c5c4)ccc32)C1]

### Preparation of (S)-2-(2-((5-(1-aminoisoquinolin-7-yl)-1-(1-(ethoxycarbonyl)pyrrolidin-3-yl)-1H-indazol-3-yl)methoxy)-6-cyclopropylphenyl)acetic acid (356e)

### Step-1: Preparation of (S)-tert-butyl 3-(5-bromo-3-((3-cyclopropyl-2-(2-ethoxy-2-oxoethyl)phenoxy)methyl)-1H-indazol-1-yl)pyrrolidine-1-carboxylate (356a)

Compound 356a was prepared according to the procedure reported in step-2 of scheme-2, from (S)-tert-butyl 3-(5-bromo-3-(hydroxymethyl)-1H-indazol-1-yl)pyrrolidine-1-carboxylate (343d) (1.649 g, 4.16 mmol) in DCM (50 mL) using ethyl 2-(2-cyclopropyl-6-hydroxyphenyl)acetate (290a) (1.1 g, 4.99 mmol), PPh₃ (1.201 g, 4.58 mmol) and a solution of DCAD (1.681 g, 4.58 mmol) in DCM (10 mL) to afford after workup and purification using method-BR. (S)-tert-butyl 3-(5-bromo-3-((3-cyclopropyl-2-(2-ethoxy-2-oxoethyl)phenoxy)methyl)-1H-indazol-1-yl)pyrrolidine-1-carboxylate (356a) (1.620 g, 65% yield) as a white solid; MS (ES+): 620.2 (M+Na).

### Step-2: Preparation of (S)-ethyl 2-(2-((5-bromo-1-(pyrrolidin-3-yl)-1H-indazol-3-yl)methoxy)-6-cyclopropylphenyl)acetate (356b)

Compound 356b was prepared according to the procedure reported in step-4 of scheme-9, from (S)-tert-butyl 3-(5-bromo-3-((3-cyclopropyl-2-(2-ethoxy-2-oxoethyl)phenoxy)methyl)-1H-indazol-1-yl)pyrrolidine-1-carboxylate (356a) (1.520 g, 2.54 mmol) in DCM (60 mL) using TFA (1.957 mL, 25.4 mmol) and stirring at RT for 16 h. The reaction mixture was concentrated, and the residue was neutralized with sodium bicarbonate and extracted with ethyl acetate (2×50 mL). The combined organic layer was washed with water (100 mL), brine (50 mL) dried and concentrated in vacuum to afford (S)-ethyl 2-(2-((5-bromo-1-(pyrrolidin-3-yl)-1H-indazol-3-yl)methoxy)-6-cyclopropylphenyl)acetate (356b) (560 mg, 44% yield) and was used as such without further purification; MS (ES+): 498.1 (M+1).

### Step-3: Preparation of (S)-ethyl 3-(5-bromo-3-((3-cyclopropyl-2-(2-ethoxy-2-oxoethyl)phenoxy)methyl)-1H-indazol-1-yl)pyrrolidine-1-carboxylate (356c)

Compound 356c was prepared according to the procedure reported in step-1 of scheme-126, from (S)-ethyl 2-(2-((5-bromo-1-(pyrrolidin-3-yl)-1H-indazol-3-yl)methoxy)-6-cyclopropylphenyl)acetate (356b) (555 mg, 1.114 mmol) in THF (5 mL) and water (5 mL) using sodium bicarbonate (140 mg, 1.670 mmol) and ethyl carbonochloridate (181 mg, 1.670 mmol) to afford after workup and purification using method-J. (S)-ethyl 3-(5-bromo-3-((3-cyclopropyl-2-(2-ethoxy-2-oxoethyl)phenoxy)methyl)-1H-indazol-1-yl)pyrrolidine-1-carboxylate (356c) (453 mg, 71% yield) as a clear oil; MS (ES+): 570.2 (M+1).

### Step-4: Preparation of (S)-ethyl 3-(5-(1-aminoisoquinolin-7-yl)-3-((3-cyclopropyl-2-(2-ethoxy-2-oxoethyl)phenoxy)methyl)-1H-indazol-1-yl)pyrrolidine-1-carboxylate (356d)

Compound 356d was prepared according to the procedure reported in step-5 of scheme-1, from (S)-ethyl 3-(5-bromo-3-((3-cyclopropyl-2-(2-ethoxy-2-oxoethyl)phenoxy)methyl)-1H-indazol-1-yl)pyrrolidine-1-carboxylate (356c) (225 mg, 0.394 mmol) in Dioxane/MeTHF (10 mL; ratio 9:1) using (1-aminoisoquinolin-7-yl)boronic acid (87a) (96 mg, 0.513 mmol), K₃PO₄ (4M aqueous solution, 394 μl, 1.578 mmol). PCy₃ (22.12 mg, 0.079 mmol). Pd₂(dba)₃ (36.1 mg, 0.039 mmol) and PdCl₂(dppf)-CH₂Cl₂Adduct (32.2 mg, 0.039 mmol) to afford after workup and purification using method-AE. (S)-ethyl 3-(5-(1-aminoisoquinolin-7-yl)-3-((3-cyclopropyl-2-(2-ethoxy-2-oxoethyl)phenoxy)methyl)-1H-indazol-1-yl)pyrrolidine-1-carboxylate (356d) (115 mg, 46% yield) as a yellow solid; MS (ES+): 634.3 (M+1).

### Step-7: Preparation of (S)-2-(2-((5-(1-aminoisoquinolin-7-yl)-1-(1-(ethoxycarbonyl)pyrrolidin-3-yl)-1H-indazol-3-yl)methoxy)-6-cyclopropylphenyl)acetic acid (356e)

Compound 356e was prepared according to the procedure reported in step-2 of scheme-1, from (S)-ethyl 3-(5-(1-aminoisoquinolin-7-yl)-3-((3-cyclopropyl-2-(2-ethoxy-2-oxoethyl)phenoxy)methyl)-1H-indazol-1-yl)pyrrolidine-1-carboxylate (356d) (115 mg, 0.181 mmol) in THF (5 mL) using lithium hydroxide hydrate (22.84 mg, 0.544 mmol) in water (1 mL) and stirring overnight at RT to afford after work up and purification using method-M, (S)-2-(2-((5-(1-aminoisoquinolin-7-yl)-1-(1-(ethoxycarbonyl)pyrrolidin-3-yl)-1H-indazol-3-yl)methoxy)-6-cyclopropylphenyl)acetic acid (356e) (38.5 mg, 35% yield) HCl salt as a white solid; ¹H NMR (300 MHz, DMSO-d₆) δ 13.31 (s, 1H, D₂O exchangeable), 12.11 (s, 1H, D₂O exchangeable), 9.15 (s, 2H, D₂O exchangeable), 8.96 (s, 1H), 8.39 (dd, J=8.6, 1.6 Hz, 1H), 8.30 (d, J=1.6 Hz, JH), 8.09-7.99 (m, 2H), 7.98-7.90 (m, 1H), 7.69 (d, J=6.9 Hz, 1H), 7.27 (d, J=6.9 Hz, 1H), 7.21-7.11 (m, 2H), 6.71-6.62 (m, 1H), 5.62-5.50 (m, 1H), 5.46 (s, 2H), 4.15-3.98 (m, 2H), 3.95-3.83 (m, 1H), 3.76 (s, 2H), 3.73-3.60 (m, 2H), 3.59-3.45 (m, 1H), 2.47-2.30 (m, 2H), 1.89-1.76 (m, 1H), 1.27-1.10 (m, 3H), 0.90-0.80 (m, 2H), 0.61-0.49 (m, 2H); MS (ES+): 606.3 (M+1); (ES−): 604.2 (M−1); Optical Rotation: +3.636 [0.055, MeOH].

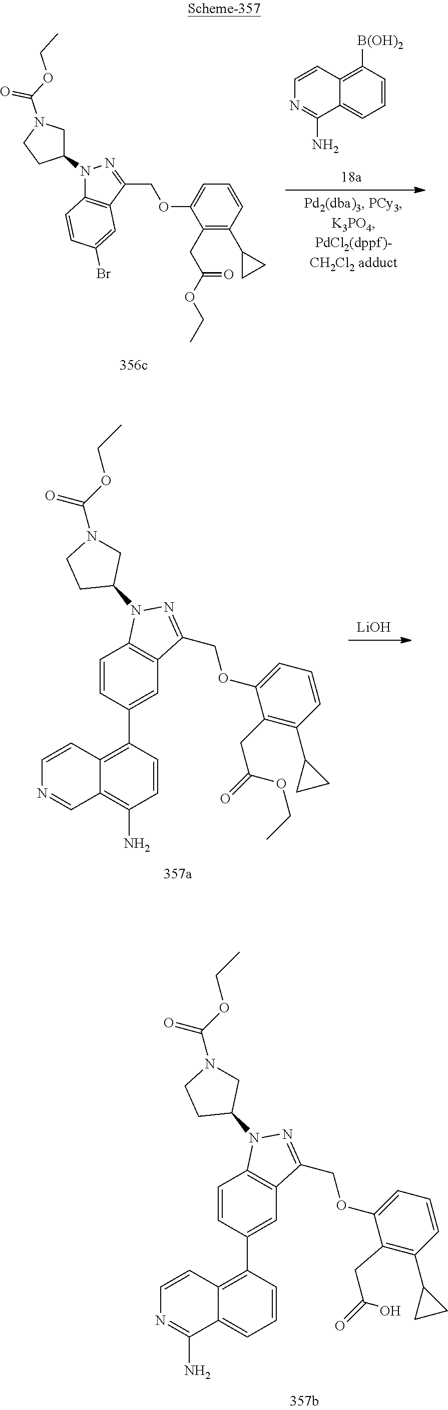 [SMILES: CCOC(=O)Cc1c(OCc2nn([C@H]3CCN(C(=O)OCC)C3)c3ccc(Br)cc23)cccc1C1CC1 ; Nc1nccc2c(B(O)O)cccc12 ; CCOC(=O)Cc1c(OCc2nn([C@H]3CCN(C(=O)OCC)C3)c3ccc(-c4ccc(N)c5cnccc45)cc23)cccc1C1CC1 ; "LiOH" ; CCOC(=O)N1CC[C@H](n2nc(COc3cccc(C4CC4)c3CC(=O)O)c3cc(-c4cccc5c(N)nccc45)ccc32)C1]

### Preparation of (S)-2-(2-((5-(1-aminoisoquinolin-5-yl)-1-(1-(ethoxycarbonyl)pyrrolidin-3-yl)-1H-indazol-3-yl)methoxy)-6-cyclopropylphenyl)acetic acid (357b)

### Step-1: Preparation of (S)-ethyl 3-(5-(1-aminoisoquinolin-5-yl)-3-((3-cyclopropyl-2-(2-ethoxy-2-oxoethyl)phenoxy)methyl)-1H-indazol-1-yl)pyrrolidine-1-carboxylate (357a)

Compound 357a was prepared according to the procedure reported in step-5 of scheme-1, from (S)-ethyl 3-(5-bromo-3-((3-cyclopropyl-2-(2-ethoxy-2-oxoethyl)phenoxy)methyl)-1H-indazol-1-yl)pyrrolidine-1-carboxylate (356c) (225 mg, 0.394 mmol) in Dioxane/MeTHF (10 mL; ratio 9:1) using (1-aminoisoquinolin-5-yl)boronic acid (18a) (96 mg, 0.513 mmol), K₃PO₄ (2M aqueous solution, 335 mg, 1.578 mmol), PCy₃ (22.12 mg, 0.079 mmol), Pd₂(dba)₃ (36.1 mg, 0.039 mmol) and PdCl₂(dppf)-CH₂Cl₂Adduct (32.2 mg, 0.039 mmol) to afford after workup and purification using method-AE, (S)-ethyl 3-(5-(1-aminoisoquinolin-5-yl)-3-((3-cyclopropyl-2-(2-ethoxy-2-oxoethyl)phenoxy)methyl)-1H-indazol-1-yl)pyrrolidine-1-carboxylate (357a) (111 mg, 44% yield) as a yellow solid; MS (ES+): 634.3 (M+1).

### Step-2: Preparation of (S)-2-(2-((5-(1-aminoisoquinolin-5-yl)-1-(1-(ethoxycarbonyl)pyrrolidin-3-yl)-1H-indazol-3-yl)methoxy)-6-cyclopropylphenyl)acetic acid (357b)

Compound 357b was prepared according to the procedure reported in step-2 of scheme-1, from (S)-ethyl 3-(5-(1-aminoisoquinolin-5-yl)-3-((3-cyclopropyl-2-(2-ethoxy-2-oxoethyl)phenoxy)methyl)-1H-indazol-1-yl)pyrrolidine-1-carboxylate (357a) (111 mg, 0.175 mmol) in THF (5 mL) using lithium hydroxide hydrate (22.05 mg, 0.525 mmol) in water (1 mL) and stirring overnight at RT to afford after work up and purification using method-M, (S)-2-(2-((5-(1-aminoisoquinolin-5-yl)-1-(1-(ethoxycarbonyl)pyrrolidin-3-yl)-1H-indazol-3-yl)methoxy)-6-cyclopropylphenyl)acetic acid (357b) (18.2 mg, 17% yield) HCl salt as a white solid; ¹H NMR (300 MHz, DMSO-d₆) δ 13.30 (s, 1H, D₂O exchangeable), 11.97 (s, 1H, D₂O exchangeable), 9.13 (s, 2H, D₂O exchangeable), 8.63 (d, J=8.2 Hz, 1H), 7.99-7.81 (m, 4H), 7.64 (d, J=7.3 Hz, 1H), 7.52 (dd, J=8.7, 1.6 Hz, 1H), 7.19-7.07 (m, 2H), 6.98 (d, J=7.2 Hz, 1H), 6.63 (dd, J=6.3, 2.5 Hz, 1H), 5.63-5.50 (m, 1H), 5.43 (s, 2H), 4.15-4.01 (m, 2H), 3.96-3.85 (m, 1H), 3.77-3.62 (m, 4H), 3.60-3.48 (m, 1H), 2.48-2.31 (m, 2H), 1.87-1.75 (m, 1H), 1.29-1.14 (m, 3H), 0.91-0.80 (m, 2H), 0.59-0.49 (m, 2H); MS (ES+): 606.3 (M+1); (ES−): 604.2 (M−1); Optical Rotation: +5.333 [0.075, MeOH].

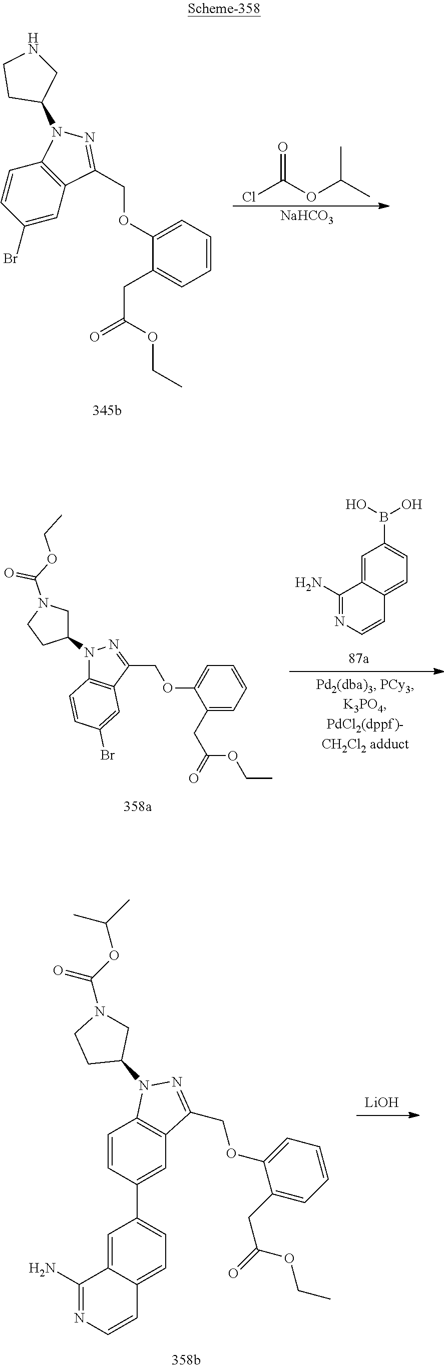

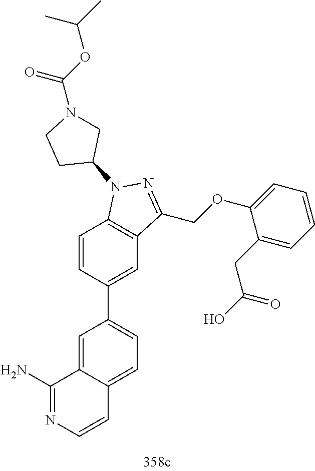 [SMILES: CC(C)OC(=O)N1CC[C@H](n2nc(COc3ccccc3CC(=O)O)c3cc(-c4ccc5ccnc(N)c5c4)ccc32)C1]

### Preparation of (S)-2-(2-((5-(1-aminoisoquinolin-7-yl)-1-(1-(isopropoxycarbonyl)pyrrolidin-3-yl)-1H-indazol-3-yl)methoxy)phenyl)acetic acid (358c)

### Step-1: Preparation of (S)-isopropyl 3-(5-bromo-3-((2-(2-ethoxy-2-oxoethyl)phenoxy)methyl)-1H-indazol-1-yl)pyrrolidine-1-carboxylate (358a)

Compound 358a was prepared according to the procedure reported in step-1 of scheme-126, from

(S)-ethyl 2-(2-((5-bromo-1-(pyrrolidin-3-yl)-1H-indazol-3-yl)methoxy)phenyl)acetate (345b) (480 mg, 1.047 mmol) in THF (5 mL) and water (5 mL) using sodium bicarbonate (132 mg, 1.571 mmol) and isopropyl carbonochloridate (128 mg, 1.047 mmol) to afford after workup and purification using method-J, (S)-isopropyl 3-(5-bromo-3-((2-(2-ethoxy-2-oxoethyl)phenoxy)methyl)-1H-indazol-1-yl)pyrrolidine-1-carboxylate (358a) (427 mg, 75% yield) as a clear oil; MS (ES+): 544.1 (M+1).

### Step-2: Preparation of (S)-isopropyl 3-(5-(1-aminoisoquinolin-7-yl)-3-((2-(2-ethoxy-2-oxoethyl)phenoxy)methyl)-1H-indazol-1-yl)pyrrolidine-1-carboxylate (358b)

Compound 358b was prepared according to the procedure reported in step-5 of scheme-1, from (S)-isopropyl 3-(5-bromo-3-((2-(2-ethoxy-2-oxoethyl)phenoxy)methyl)-1H-indazol-1-yl)pyrrolidine-1-carboxylate (358a) (210 mg, 0.386 mmol) in dioxane/MeTHF (10 mL; ratio 9:1) using (1-aminoisoquinolin-7-yl)boronic acid (87a) (94 mg, 0.501 mmol), K₃PO₄ (4M aqueous solution, 0.386 mL, 0.154 mmol), PCy₃ (21.63 mg, 0.077 mmol), Pd₂(dba)₃ (35.3 g, 0.039 mmol) and PdCl₂(dppf)-CH₂Cl₂Adduct (31.5 mg, 0.039 mmol) to afford after workup and purification using method-AE. (S)-isopropyl 3-(5-(1-aminoisoquinolin-7-yl)-3-((2-(2-ethoxy-2-oxoethyl)phenoxy)methyl)-1H-indazol-1-yl)pyrrolidine-1-carboxylate (358b) (162 mg, 69% yield) as a yellow solid; MS (ES+): 608.3 (M+1).

### Step-3: Preparation of (S)-2-(2-((5-(1-aminoisoquinolin-7-yl)-1-(1-(isopropoxycarbonyl)pyrrolidin-3-yl)-1H-indazol-3-yl)methoxy)phenyl)acetic acid (358c)

Compound 358c was prepared according to the procedure reported in step-2 of scheme-1, from (S)-isopropyl 3-(5-(1-aminoisoquinolin-7-yl)-3-((2-(2-ethoxy-2-oxoethyl)phenoxy)methyl)-1H-indazol-1-yl)pyrrolidine-1-carboxy late (358b) (162 mg, 0.267 mmol) in THF (5 mL) using lithium hydroxide hydrate (33.6 mg, 0.800 mmol) in water (1 mL) and stirring overnight at RT to afford after work up and purification using method-M. (S)-2-(2-((5-(1-aminoisoquinolin-7-yl)-1-(1-(isopropoxycarbonyl)pyrrolidin-3-yl)-1H-indazol-3-yl)methoxy)phenyl)acetic acid (358c) (68.2 mg, 44% yield) HCl salt as a white solid; ¹H NMR (300 MHz, DMSO-d₆) δ 13.35 (s, 1H, D₂O exchangeable), 12.15 (s, 1H, D₂O exchangeable), 9.18 (s, 2H, D₂O exchangeable), 8.98 (d, J=1.8 Hz, 1H), 8.42 (dd, J=8.5, 1.6 Hz, 1H), 8.34 (d, J=1.6 Hz, 1H), 8.05 (dd, J=8.8, 1.5 Hz, 2H), 7.99-7.90 (m, 1H), 7.70 (d, J=6.9 Hz, 1H), 7.34-7.24 (m, 3H), 7.24-7.18 (m, 1H), 6.97-6.87 (m, 1H), 5.63-5.52 (m, 1H), 5.49 (s, 2H), 4.88-4.74 (m, 1H), 3.94-3.83 (m, 1H), 3.73-3.60 (m, 2H), 3.58-3.46 (m, 3H), 2.48-2.30 (m, 2H), 1.30-1.12 (m, 6H); MS (ES+): 580.3 (M+1); (ES−): 578.2 (M−1); Optical Rotation: +7.0 [0.2, MeOH].

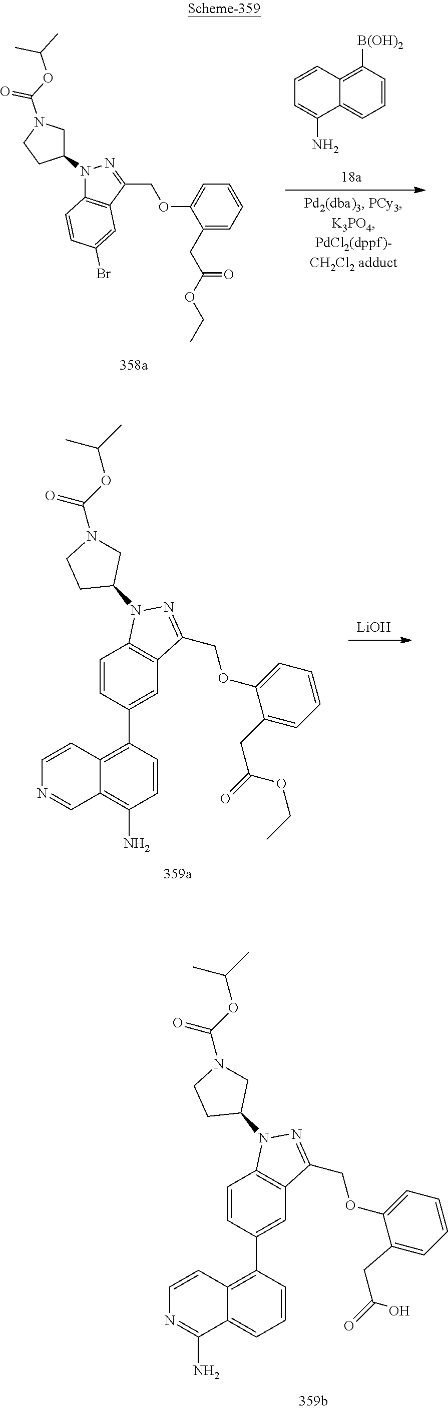 [SMILES: CCOC(=O)Cc1ccccc1OCc1nn([C@H]2CCN(C(=O)OC(C)C)C2)c2ccc(Br)cc12 ; Nc1cccc2c(B(O)O)cccc12 ; CCOC(=O)Cc1ccccc1OCc1nn([C@H]2CCN(C(=O)OC(C)C)C2)c2ccc(-c3ccc(N)c4cnccc34)cc12 ; "LiOH" ; CC(C)OC(=O)N1CC[C@H](n2nc(COc3ccccc3CC(=O)O)c3cc(-c4cccc5c(N)nccc45)ccc32)C1]

### Preparation of (S)-2-(2-((5-(1-aminoisoquinolin-5-yl)-1-(1-(isopropoxycarbonyl)pyrrolidin-3-yl)-1H-indazol-3-yl)methoxy)phenyl)acetic acid (359b)

### Step-1: Preparation of (S)-isopropyl 3-(5-(1-aminoisoquinolin-5-yl)-3-((2-(2-ethoxy-2-oxoethyl)phenoxy)methyl)-1H-indazol-1-yl)pyrrolidine-1-carboxylate (359a)

Compound 359a was prepared according to the procedure reported in step-5 of scheme-1, from (S)-isopropyl 3-(5-bromo-3-((2-(2-ethoxy-2-oxoethyl)phenoxy)methyl)-1H-indazol-1-yl)pyrrolidine-1-carboxylate (358a) (210 mg, 0.386 mmol) in dioxane/MeTHF (10 mL; ratio 9:1) using (1-aminoisoquinolin-5-yl)boronic acid (18a) (94 g, 501 mmol), K₃PO₄ (2 M aqueous solution, 327 mg, 1.543 mmol), PCy₃ (21.63 mg, 0.077 mmol), Pd₂(dba)₃ (35.3 mg, 0.039 mmol) and PdCl₂(dppf)-CH₂Cl₂Adduct (31.5 mg, 0.039 mmol) to afford after workup and purification using method-AE, (S)-isopropyl 3-(5-(1-aminoisoquinolin-5-yl)-3-((2-(2-ethoxy-2-oxoethyl)phenoxy)methyl)-1H-indazol-1-yl)pyrrolidine-1-carboxylate (359a) (152 mg, 65% yield) as a yellow solid; MS (ES+): 608.3 (M+1).

### Step-2: Preparation of (S)-2-(2-((5-(1-aminoisoquinolin-5-yl)-1-(1-(isopropoxycarbonyl)pyrrolidin-3-yl)-1H-indazol-3-yl)methoxy)phenyl)acetic acid (359b)

Compound 359b was prepared according to the procedure reported in step-2 of scheme-1, from (S)-isopropyl 3-(5-(1-aminoisoquinolin-5-yl)-3-((2-(2-ethoxy-2-oxoethyl)phenoxy)methyl)-1H-indazol-1-yl)pyrrolidine-1-carboxylate (359a) (152 mg, 0.25 mmol) in THF (5 mL) using lithium hydroxide hydrate (31.5 mg, 0.75 mmol) in water (1 mL) and stirring overnight at RT to afford after work up and purification using method-M, (S)-2-(2-((5-(1-aminoisoquinolin-5-yl)-1-(1-(isopropoxycarbonyl)pyrrolidin-3-yl)-1H-indazol-3-yl)methoxy)phenyl)acetic acid (359b) (44.1 mg, 30% yield) HCl salt as a white solid; ¹H NMR (300 MHz, DMSO-d₆) δ 13.25 (s, 1H, D₂O exchangeable), 11.99 (s, 1H, D₂O exchangeable), 9.11 (s, 2H, D₂O exchangeable), 8.61 (d, J=8.3 Hz, 1H), 7.99-7.79 (m, 4H), 7.62 (d, J=7.2 Hz, 1H), 7.51 (dd, J=8.6, 1.7 Hz, 1H), 7.30-7.21 (m, 2H), 7.21-7.14 (m, 1H), 6.98 (d, J=7.3 Hz, 1H), 6.95-6.86 (m, 1H), 5.62-5.50 (m, 1H), 5.45 (s, 2H), 4.89-4.74 (m, 1H), 3.95-3.83 (m, 1H), 3.74-3.59 (m, 2H), 3.59-3.50 (m, 1H), 3.47 (s, 2H), 2.47-2.32 (m, 2H), 1.29-1.10 (m, 6H); MS (ES+): 580.2 (M+1); (ES−): 578.2 (M−1); Optical Rotation: +5.333 [0.075, MeOH].

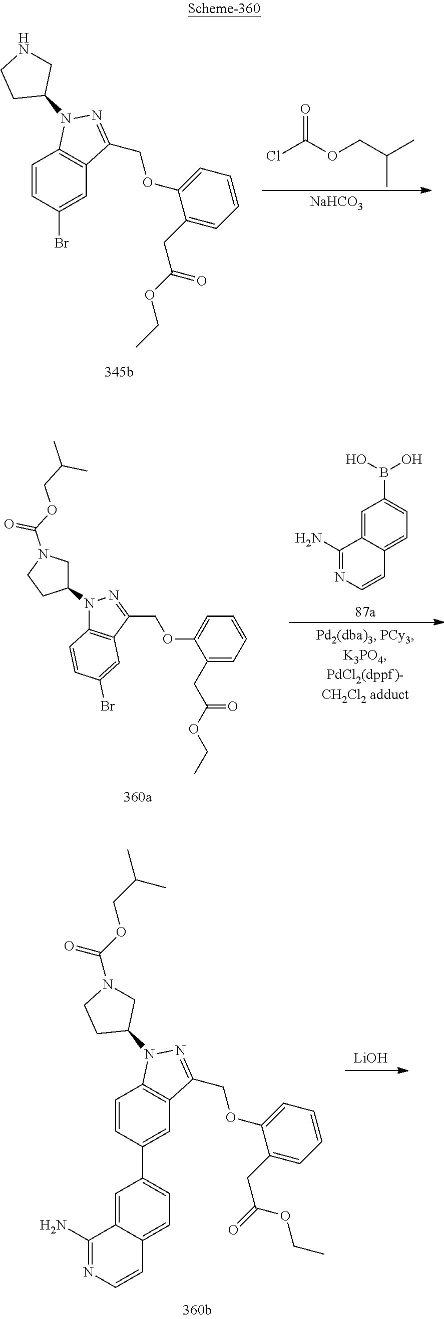

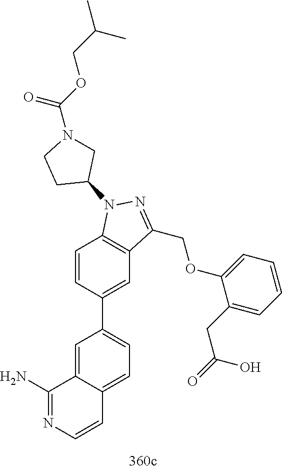 [SMILES: CC(C)COC(=O)N1CC[C@H](n2nc(COc3ccccc3CC(=O)O)c3cc(-c4ccc5ccnc(N)c5c4)ccc32)C1]

### Preparation of (S)-2-(2-((5-(1-aminoisoquinolin-7-yl)-1-(1-(isobutoxycarbonyl)pyrrolidin-3-yl)-1H-indazol-3-yl)methoxy)phenyl)acetic acid (360c)

### Step-1: Preparation of (S)-isobutyl 3-(5-bromo-3-((2-(2-ethoxy-2-oxoethyl)phenoxy)methyl)-1H-indazol-1-yl)pyrrolidine-1-carboxylate (360a)

Compound 360a was prepared according to the procedure reported in step-1 of scheme-126, from (S)-ethyl 2-(2-((5-bromo-1-(pyrrolidin-3-yl)-1H-indazol-3-yl)methoxy)phenyl)acetate (345b) (480 mg, 1.047 mmol) in THF (5 mL) and water (5 mL) using sodium bicarbonate (132 mg, 1.571 mmol) and isobutyl carbonochloridate (143 mg, 1.047 mmol) to afford after workup and purification using method-J. (S)-isobutyl 3-(5-bromo-3-((2-(2-ethoxy-2-oxoethyl)phenoxy)methyl)-1H-indazol-1-yl)pyrrolidine-1-carboxylate (360a) (385 mg, 66% yield) as a clear oil; MS (ES+): 558.2 (M+1).

### Step-2: Preparation of (S)-isobutyl 3-(5-(1-aminoisoquinolin-7-yl)-3-((2-(2-ethoxy-2-oxoethyl)phenoxy)methyl)-1H-indazol-1-yl)pyrrolidine-1-carboxylate (360b)

Compound 360b was prepared according to the procedure reported in step-5 of scheme-1, from (S)-isobutyl 3-(5-bromo-3-((2-(2-ethoxy-2-oxoethyl)phenoxy)methyl)-1H-indazol-1-yl)pyrrolidine-1-carboxylate (360a) (190 mg, 0.340 mmol) in dioxane/MeTHF (10 mL; ratio 9:1) using (1-aminoisoquinolin-7-yl)boronic acid (87a) (83 mg, 0.442 mmol), K₃PO₄ (4M aqueous solution, 340 μl, 1.361 mmol), PCy₃ (19.08 mg, 0.068 mmol). Pd₂(dba)₃ (31.2 mg, 0.034 mmol) and PdCl₂(dppf)-CH₂Cl₂Adduct (27.8 mg, 0.034 mmol) to afford after workup and purification using method-AE, (S)-isobutyl 3-(5-(1-aminoisoquinolin-7-yl)-3-((2-(2-ethoxy-2-oxoethyl)phenoxy)methyl)-1H-indazol-1-yl)pyrrolidine-1-carboxylate (360b) (142 mg, 67% yield) as a yellow solid; MS (ES+): 622.3 (M+1).

### Step-3: Preparation of (S)-2-(2-((5-(1-aminoisoquinolin-7-yl)-1-(1-(isobutoxycarbonyl)pyrrolidin-3-yl)-1H-indazol-3-yl)methoxy)phenyl)acetic acid (360c)

Compound 360c was prepared according to the procedure reported in step-2 of scheme-1, from (S)-isobutyl 3-(5-(1-aminoisoquinolin-7-yl)-3-((2-(2-ethoxy-2-oxoethyl)phenoxy)methyl)-1H-indazol-1-yl)pyrrolidine-1-carboxylate (360b) (142 mg, 0.228 mmol) in THF (5 mL) using lithium hydroxide hydrate (28.8 mg, 0.685 mmol) in water (1 mL) and stirring overnight at RT to afford after work up and purification using method-M. (S)-2-(2-((5-(1-aminoisoquinolin-7-yl)-1-(1-(isobutoxycarbonyl)pyrrolidin-3-yl)-1H-indazol-3-yl)methoxy)phenyl)acetic acid (360c) (21.4 mg, 16% yield) HCl salt as a white solid; ¹H NMR (300 MHz, DMSO-d₆) δ 13.18 (s, 1H, D₂O exchangeable), 12.07 (s, 1H, D₂O exchangeable), 9.09 (s, 2H, D₂O exchangeable), 8.95 (s, 1H), 8.41 (dd. J=8.5, 1.7 Hz, 1H), 8.32 (d, J=1.6 Hz, 1H), 8.09-8.00 (m, 2H), 7.98-7.91 (m, 1H), 7.69 (d, J=6.9 Hz, 1H), 7.33-7.24 (m, 3H), 7.23-7.16 (m, 1H), 6.96-6.86 (m, 1H), 5.63-5.51 (m, 1H), 5.48 (s, 2H), 4.00-3.88 (m, 1H), 3.87-3.80 (m, 2H), 3.75-3.64 (m, 2H), 3.61-3.46 (m, 3H), 2.46-2.29 (m, 2H), 1.95-1.82 (m, 1H), 0.99-0.81 (m, 6H); MS (ES+): 594.3 (M+1); (ES−): 592.2 (M−1); Optical Rotation: +4.0 [0.05. MeOH].

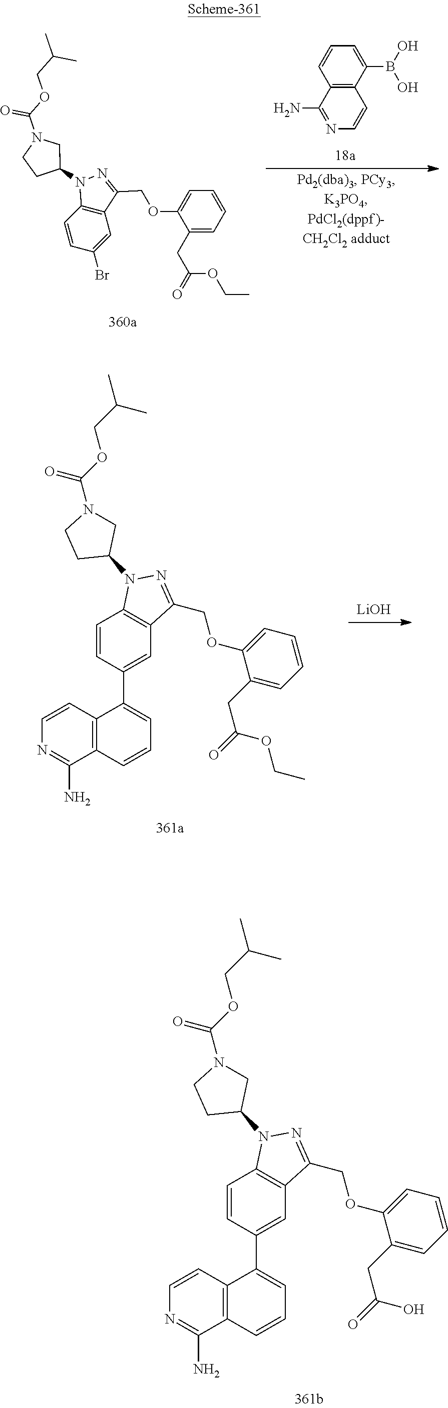 [SMILES: CCOC(=O)Cc1ccccc1OCc1nn([C@H]2CCN(C(=O)OCC(C)C)C2)c2ccc(Br)cc12 ; Nc1nccc2c(B(O)O)cccc12 ; CCOC(=O)Cc1ccccc1OCc1nn([C@H]2CCN(C(=O)OCC(C)C)C2)c2ccc(-c3cccc4c(N)nccc34)cc12 ; "LiOH" ; CC(C)COC(=O)N1CC[C@H](n2nc(COc3ccccc3CC(=O)O)c3cc(-c4cccc5c(N)nccc45)ccc32)C1]

### Preparation of (S)-2-(2-((5-(1-aminoisoquinolin-5-yl)-1-(1-(isobutoxycarbonyl)pyrrolidin-3-yl)-1H-indazol-3-yl)methoxy)phenyl)acetic acid (361b)

### Step-1: Preparation of (S)-isobutyl 3-(5-(1-aminoisoquinolin-5-yl)-3-((2-(2-ethoxy-2-oxoethyl)phenoxy)methyl)-1H-indazol-1-yl)pyrrolidine-1-carboxylate (361a)

Compound 361a was prepared according to the procedure reported in step-5 of scheme-1, from (S)-isobutyl 3-(5-bromo-3-((2-(2-ethoxy-2-oxoethyl)phenoxy)methyl)-1H-indazol-1-yl)pyrrolidine-1-carboxylate (360a) (190 mg, 0.340 mmol) in dioxane/MeTHF (10 mL; ratio 9:1) using (1-aminoisoquinolin-5-yl)boronic acid (18a) (64.0 mg, 0.340 mmol), K₃PO₄ (2 M aqueous solution, 289 mg, 1.361 mmol), PCy₃ (19.08 mg, 0.068 mmol), Pd₂(dba)₃ (31.2 mg, 0.034 mmol) and PdCl₂(dppf)-CH₂Cl₂Adduct (27.8 mg, 0.034 mmol) to afford after workup and purification using method-AE, (S)-isobutyl 3-(5-(1-aminoisoquinolin-5-yl)-3-((2-(2-ethoxy-2-oxoethyl)phenoxy)methyl)-1H-indazol-1-yl)pyrrolidine-1-carboxylate (361a) (131 mg, 62% yield) as a yellow solid; MS (ES+): 622.3 (M+1).

### Step-2: Preparation of (S)-2-(2-((5-(1-aminoisoquinolin-5-yl)-1-(1-(isobutoxycarbonyl)pyrrolidin-3-yl)-1H-indazol-3-yl)methoxy)phenyl)acetic acid (361b)

Compound 361b was prepared according to the procedure reported in step-2 of scheme-1, from (S)-isobutyl 3-(5-(1-aminoisoquinolin-5-yl)-3-((2-(2-ethoxy-2-oxoethyl)phenoxy)methyl)-1H-indazol-1-yl)pyrrolidine-1-carboxylate (361a) (131 mg, 0.211 mmol) in THF (5 mL) using lithium hydroxide hydrate (26.5 mg, 0.632 mmol) in water (1 mL) and stirring overnight at RT to afford after work up and purification using method-M, (S)-2-(2-((5-(1-aminoisoquinolin-5-yl)-1-(1-(isobutoxycarbonyl)pyrrolidin-3-yl)-1H-indazol-3-yl)methoxy)phenyl)acetic acid (361b) (34.2 mg, 27% yield) HCl salt as a white solid; 1H NMR (300 MHz, DMSO-d₆) δ 13.20 (s, 1H, D₂O exchangeable), 11.97 (s, 1H, DO exchangeable), 9.07 (s, 2H, D₂O) exchangeable), 8.61 (d, J=8.2 Hz, 1H), 7.98-7.80 (m, 4H), 7.62 (d, J=7.2 Hz, 1H), 7.51 (dd. J=8.7, 1.7 Hz, 1H), 7.27-7.20 (m, 2H), 7.20-7.13 (m, 1H), 6.98 (d, J=7.2 Hz, 1H), 6.94-6.85 (m, 1H), 5.63-5.50 (m, 1H), 5.45 (s, 2H), 3.97-3.87 (m, 1H), 3.86-3.78 (m, 2H), 3.77-3.65 (m, 2H), 3.58-3.50 (m, 1H), 3.47 (s, 2H), 2.48-2.35 (m, 2H), 1.99-1.81 (m, 1H), 0.96-0.83 (m, 6H); MS (ES+): 594.3 (M+1); (ES−): 592.2 (M−1); Optical Rotation: +8.0 [0.05, MeOH].

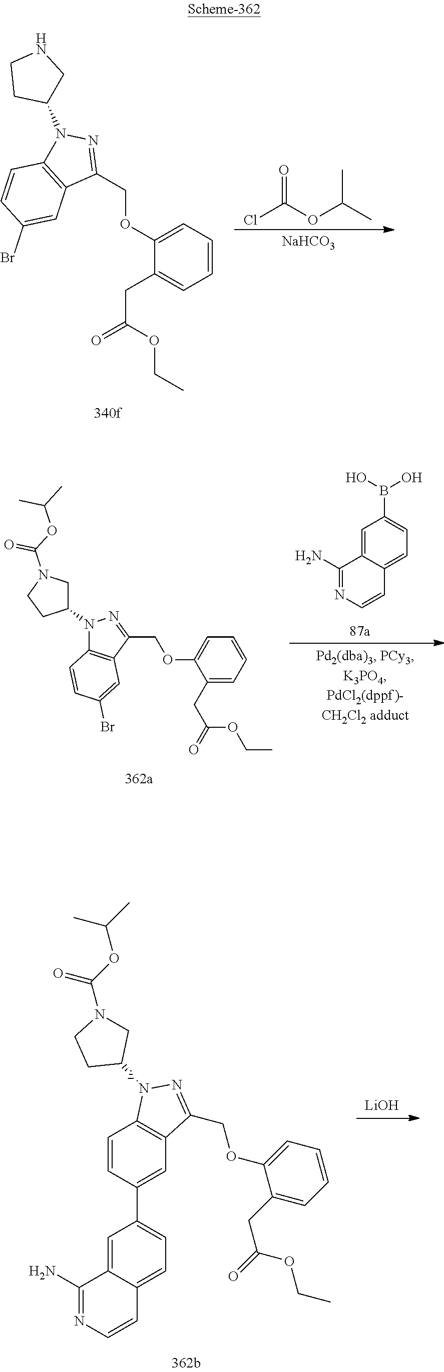

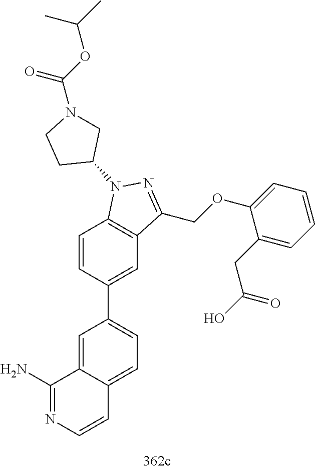 [SMILES: CC(C)OC(=O)N1CC[C@@H](n2nc(COc3ccccc3CC(=O)O)c3cc(-c4ccc5ccnc(N)c5c4)ccc32)C1]

### Preparation of (R)-2-(2-((5-(1-aminoisoquinolin-7-yl)-1-(1-(isopropoxycarbonyl)pyrrolidin-3-yl)-1H-indazol-3-yl)methoxy)phenyl)acetic acid (362c)

### Step-1: Preparation of (R)-isopropyl 3-(5-bromo-3-((2-(2-ethoxy-2-oxoethyl)phenoxy)methyl)-1H-indazol-1-yl)pyrrolidine-1-carboxylate (362a)

Compound 362a was prepared according to the procedure reported in step-1 of scheme-126, from (R)-ethyl 2-(2-((5-bromo-1-(pyrrolidin-3-yl)-1H-indazol-3-yl)methoxy)phenyl)acetate (340f) (675 mg, 1.473 mmol) in THF (5 mL) and water (5 mL) using sodium bicarbonate (186 mg, 2.209 mmol) and isopropyl carbonochloridate (180 mg, 1.473 mmol) to afford after workup and purification using method-J, (R)-isopropyl 3-(5-bromo-3-((2-(2-ethoxy-2-oxoethyl)phenoxy)methyl)-1H-indazol-1-yl)pyrrolidine-1-carboxylate (362a) (595 mg, 74% yield) as a clear oil; MS (ES+): 544.1 (M+1).

### Step-2: Preparation of (R)-isopropyl 3-(5-(1-aminoisoquinolin-7-yl)-3-((2-(2-ethoxy-2-oxoethyl)phenoxy)methyl)-1H-indazol-1-yl)pyrrolidine-1-carboxylate (362b)

Compound 358b was prepared according to the procedure reported in step-5 of scheme-1, from (R)-isopropyl 3-(5-bromo-3-((2-(2-ethoxy-2-oxoethyl)phenoxy)methyl)-1H-indazol-1-yl)pyrrolidine-1-carboxylate (362a) (280 mg, 0.514 mmol) in dioxane/MeTHF (10 mL; ratio 9:1) using (1-aminoisoquinolin-7-yl)boronic acid (87a) (126 mg, 0.669 mmol), K₃PO₄ (4 M aqueous solution, 514 μl, 2.057 mmol), PCy₃ (28.8 mg, 0.103 mmol), Pd₂(dba)₃ (47.1 mg, 0.051 mmol) and PdCl₂(dppf)-CH₂Cl₂Adduct (42.0 mg, 0.051 mmol) to afford after workup and purification using method-AE, (R)-isopropyl 3-(5-(1-aminoisoquinolin-7-yl)-3-((2-(2-ethoxy-2-oxoethyl)phenoxy)methyl)-1H-indazol-1-yl)pyrrolidine-1-carboxylate (362b) (124 mg, 40% yield) as a yellow solid; MS (ES+): 608.3 (M+1).

### Step-3: Preparation of (R)-2-(2-((5-(1-aminoisoquinolin-7-yl)-1-(1-(isopropoxycarbonyl)pyrrolidin-3-yl)-1H-indazol-3-yl)methoxy)phenyl)acetic acid (362c)

Compound 362c was prepared according to the procedure reported in step-2 of scheme-1, from (R)-isopropyl 3-(5-(1-aminoisoquinolin-7-yl)-3-((2-(2-ethoxy-2-oxoethyl)phenoxy)methyl)-1H-indazol-1-yl)pyrrolidine-1-carboxylate (362b) (174 mg, 0.286 mmol) in THF (5 mL) using lithium hydroxide hydrate (36.0 mg, 0.859 mmol) in water (1 mL) and stirring overnight at RT to afford after work up and purification using method-M, (R)-2-(2-((5-(1-aminoisoquinolin-7-yl)-1-(1-(isopropoxycarbonyl)pyrrolidin-3-yl)-1H-indazol-3-yl)methoxy)phenyl)acetic acid (362c) (30.7 mg, 19% yield) HCl salt as a white solid; ¹H NMR (300 MHz, DMSO-d₆) δ 13.18 (s, 1H, D₂O exchangeable), 12.07 (s, 1H, DO exchangeable), 9.11 (s, 2H, D₂O exchangeable), 8.96 (s, 1H), 8.43 (dd, J=8.5, 1.6 Hz, 1H), 8.33 (d, J=1.6 Hz, 1H), 8.09-8.00 (m, 2H), 7.99-7.92 (m, 1H), 7.69 (d, J=6.9 Hz, 1H), 7.33-7.24 (m, 3H), 7.24-7.18 (m, 1H), 6.95-6.88 (m, 1H), 5.62-5.52 (m, 1H), 5.49 (s, 2H), 4.88-4.75 (m, 1H), 3.94-3.81 (m, 1H), 3.73-3.60 (m, 2H), 3.58-3.46 (m, 3H), 2.46-2.34 (m, 2H), 1.26-1.16 (m, 6H); MS (ES+): 580.3 (M+1); Optical Rotation: −6.67 [0.09, MeOH].

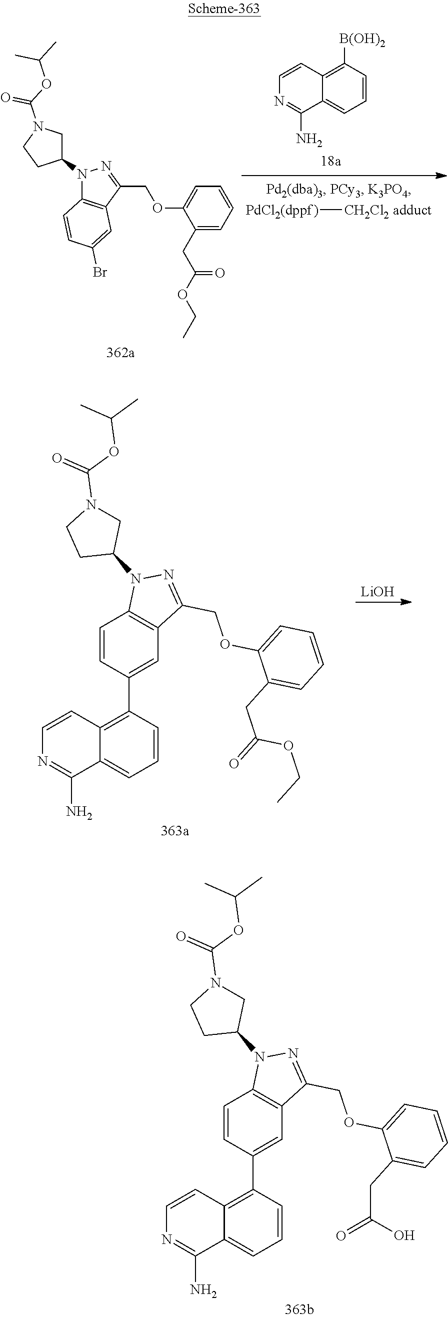

### Preparation of (R)-2-(2-((5-(1-aminoisoquinolin-5-yl)-1-(1-(isopropoxycarbonyl)pyrrolidin-3-yl)-1H-indazol-3-yl)methoxy)phenyl)acetic acid (363b)

### Step-1: Preparation of (R)-isopropyl 3-(5-(1-aminoisoquinolin-5-yl)-3-((2-(2-ethoxy-2-oxoethyl)phenoxy)methyl)-1H-indazol-1-yl)pyrrolidine-1-carboxylate (363a)

Compound 363a was prepared according to the procedure reported in step-5 of scheme-1, from (R)-isopropyl 3-(5-bromo-3-((2-(2-ethoxy-2-oxoethyl)phenoxy)methyl)-1H-indazol-1-yl)pyrrolidine-1-carboxylate (362a) (280 mg, 0.514 mmol) in dioxane/MeTHF (10 mL, ratio 9:1) using (1-aminoisoquinolin-5-yl)boronic acid (18a) (126 mg, 0.669 mmol), K₃PO₄ (2M aqueous solution, 437 mg, 2.057 mmol), PCy₃ (28.8 mg, 0.103 mmol), Pd₂(dba)₃ (47.1 mg, 0.051 mmol) and PdCl₂(dppf)-CH₂Cl₂Adduct (42.0 mg, 0.051 mmol) to afford after workup and purification using method-AE, (R)-isopropyl 3-(5-(1-aminoisoquinolin-5-yl)-3-((2-(2-ethoxy-2-oxoethyl)phenoxy)methyl)-1H-indazol-1-yl)pyrrolidine-1-carboxylate (363a) (186 mg, 60% yield) as a yellow solid; MS (ES+): 608.3 (M+1).

### Step-2: Preparation of (R)-2-(2-((5-(1-aminoisoquinolin-5-yl)-1-(1-(isopropoxycarbonyl)pyrrolidin-3-yl)-1H-indazol-3-yl)methoxy)phenyl)acetic acid (363b)

Compound 363b was prepared according to the procedure reported in step-2 of scheme-1, from (R)-isopropyl 3-(5-(1-aminoisoquinolin-5-yl)-3-((2-(2-ethoxy-2-oxoethyl)phenoxy)methyl)-1H-indazol-1-yl)pyrrolidine-1-carboxylate (363a) (186 mg, 0.306 mmol) in THF (5 mL) using lithium hydroxide hydrate (38.5 mg, 0.918 mmol) in water (1 mL) and stirring overnight at RT to afford after work up and purification using method-M, (R)-2-(2-((5-(1-aminoisoquinolin-5-yl)-1-(1-(isopropoxycarbonyl)pyrrolidin-3-yl)-1H-indazol-3-yl)methoxy)phenyl)acetic acid (363b) (79 mg, 44% yield) HCl salt as a white solid; ¹H NMR (300 MHz, DMSO-d₆) δ 13.27 (s, 1H, D₂O exchangeable), 11.98 (s, 1H, D₂O exchangeable), 9.15 (s, 2H, D₂O exchangeable), 8.62 (d, J=8.3 Hz, 1H), 7.99-7.81 (m, 4H), 7.62 (d, J=7.3 Hz, 1H), 7.51 (dd, J=8.6, 1.7 Hz, 1H), 7.28-7.21 (m, 2H), 7.20-7.14 (m, 1H), 6.98 (d, J=7.2 Hz, 1H), 6.94-6.83 (m, 1H), 5.61-5.49 (m, 1H), 5.45 (s, 2H), 4.85-4.75 (m, 1H), 3.93-3.83 (m, 1H), 3.75-3.60 (m, 2H), 3.56-3.49 (m, 1H), 3.47 (s, 2H), 2.47-2.33 (m, 2H), 1.28-1.16 (m, 6H); MS (ES+): 580.3 (M+1); (ES−): 578.2 (M−1); Optical Rotation: −1.90 [0.105, MeOH].

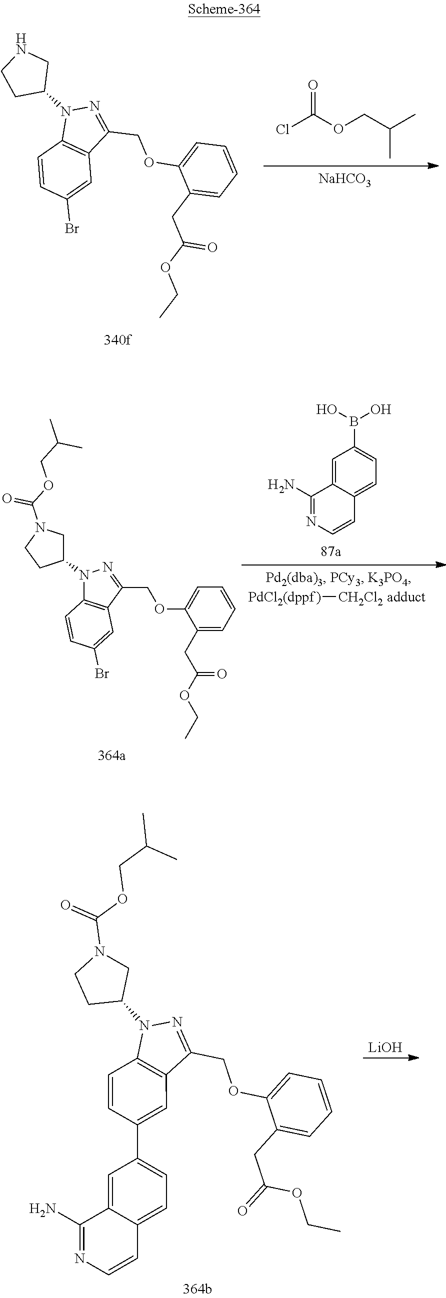

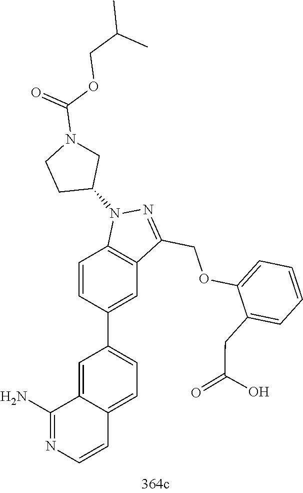 [SMILES: CC(C)COC(=O)N1CC[C@@H](n2nc(COc3ccccc3CC(=O)O)c3cc(-c4ccc5ccnc(N)c5c4)ccc32)C1]

### Preparation of (R)-2-(2-((5-(1-aminoisoquinolin-7-yl)-1-(1-(isobutoxycarbonyl)pyrrolidin-3-yl)-1H-indazol-3-yl)methoxy)phenyl)acetic acid (364c)

### Step-1: Preparation of (R)-isobutyl 3-(5-bromo-3-((2-(2-ethoxy-2-oxoethyl)phenoxy)methyl)-1H-indazol-1-yl)pyrrolidine-1-carboxylate (364a)

Compound 364a was prepared according to the procedure reported in step-1 of scheme-126, from

(R)-ethyl 2-(2-((5-bromo-1-(pyrrolidin-3-yl)-1H-indazol-3-yl)methoxy)phenyl)acetate (340f) (675 mg, 1.473 mmol) in THF (5 mL) and water (5 mL) using sodium bicarbonate (186 mg, 2.209 mmol) and isobutyl carbonochloridate (201 mg, 1.473 mmol) to afford after workup and purification using method-J, (R)-isobutyl 3-(5-bromo-3-((2-(2-ethoxy-2-oxoethyl)phenoxy)methyl)-1H-indazol-1-yl)pyrrolidine-1-carboxylate (364a) (590 mg, 72% yield) as a clear oil; MS (ES+): 558.2 (M+1).

### Step-2: Preparation of (R)-isobutyl 3-(5-(1-aminoisoquinolin-7-yl)-3-((2-(2-ethoxy-2-oxoethyl)phenoxy)methyl)-1H-indazol-1-yl)pyrrolidine-1-carboxylate (364b)

Compound 364b was prepared according to the procedure reported in step-5 of scheme-1, from (R)-isobutyl 3-(5-bromo-3-((2-(2-ethoxy-2-oxoethyl)phenoxy)methyl)-1H-indazol-1-yl)pyrrolidine-1-carboxylate (364a) (270 mg, 0.483 mmol) in dioxane/MeTHF (10 mL) using (1-aminoisoquinolin-7-yl)boronic acid (87a) (118 mg, 0.628 mmol), K₃PO₄ (4 M aqueous solution, 483 μl, 1.934 mmol), PCy₃ (27.1 mg, 0.097 mmol), Pd₂(dba)₃ (44.3 mg, 0.048 mmol) and PdCl₂(dppf)-CH₂Cl₂Adduct (40 mg, 0.048 mmol) to afford after workup and purification using method-AE, (R)-isobutyl 3-(5-(1-aminoisoquinolin-7-yl)-3-((2-(2-ethoxy-2-oxoethyl)phenoxy)methyl)-1H-indazol-1-yl)pyrrolidine-1-carboxylate (364b) (136 mg, 45% yield) as a yellow solid; MS (ES+): 622.3 (M+1).

### Step-3: Preparation of (R)-2-(2-((5-(1-aminoisoquinolin-7-yl)-1-(1-(isobutoxycarbonyl)pyrrolidin-3-yl)-1H-indazol-3-yl)methoxy)phenyl)acetic acid (364c)

Compound 364c was prepared according to the procedure reported in step-2 of scheme-1, from (R)-isobutyl 3-(5-(1-aminoisoquinolin-7-yl)-3-((2-(2-ethoxy-2-oxoethyl)phenoxy)methyl)-1H-indazol-1-yl)pyrrolidine-1-carboxylate (364b) (136 mg, 0.219 mmol) in THF (5 mL) using lithium hydroxide hydrate (28 mg, 0.656 mmol) in water (1 mL) and stirring overnight at RT to afford after work up and purification using method-M, (R)-2-(2-((5-(1-aminoisoquinolin-7-yl)-1-(1-(isobutoxycarbonyl)pyrrolidin-3-yl)-1H-indazol-3-yl)methoxy)phenyl)acetic acid (364c) (6.4 mg, 5% yield) HCl salt as a white solid; ¹H NMR (300 MHz, DMSO-d₆) δ 13.31 (s, 1H, D₂O exchangeable), 12.11 (s, 1H, D₂O exchangeable), 9.16 (s, 2H, D₂O exchangeable), 8.98 (s, 1H), 8.42 (dd, J=8.5, 1.6 Hz, 1H), 8.34 (s, 1H), 8.05 (d, J=8.6 Hz, 2H), 7.99-7.90 (m, 1H), 7.70 (d, J=6.9 Hz, 1H), 7.32-7.24 (m, 3H), 7.24-7.17 (m, 1H), 6.99-6.87 (m, 1H), 5.64-5.52 (m, 1H), 5.49 (s, 2H), 3.98-3.88 (m, 1H), 3.88-3.79 (m, 2H), 3.77-3.63 (m, 2H), 3.60-3.47 (m, 3H), 2.47-2.35 (m, 2H), 1.95-1.83 (m, 1H), 0.98-0.85 (m, 6H); MS (ES+): 594.3 (M+1); (ES−): 592.2 (M−1); Optical Rotation: −6.67 [0.06, MeOH].

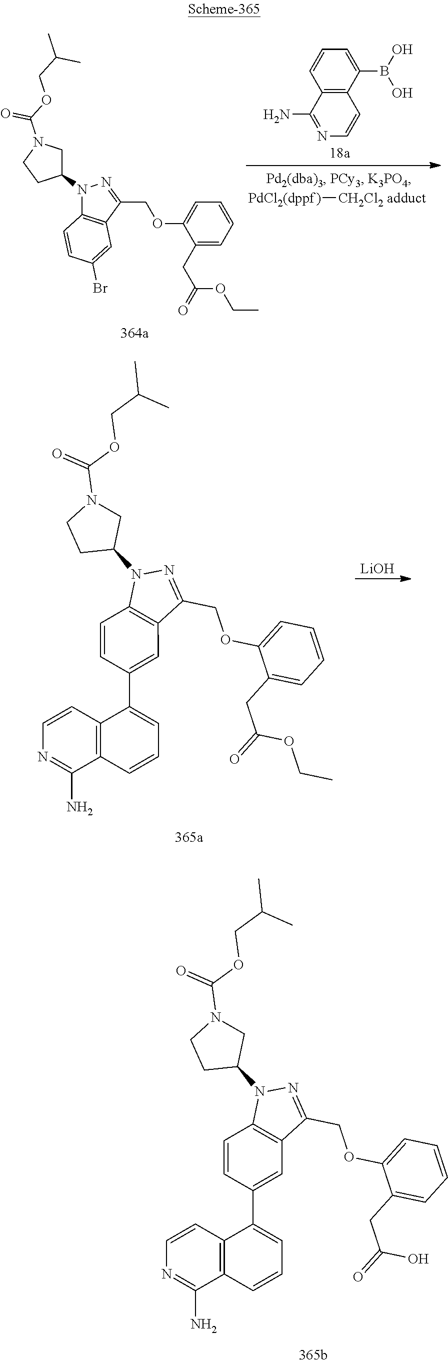

### Preparation of (R)-2-(2-((5-(1-aminoisoquinolin-5-yl)-1-(1-(isobutoxycarbonyl)pyrrolidin-3-yl)-1H-indazol-3-yl)methoxy)phenyl)acetic acid (365b)

### Step-1: Preparation of (R)-isobutyl 3-(5-(1-aminoisoquinolin-5-yl)-3-((2-(2-ethoxy-2-oxoethyl)phenoxy)methyl)-1H-indazol-1-yl)pyrrolidine-1-carboxylate (365a)

Compound 365a was prepared according to the procedure reported in step-5 of scheme-1, from (R)-isobutyl 3-(5-bromo-3-((2-(2-ethoxy-2-oxoethyl)phenoxy)methyl)-1H-indazol-1-yl)pyrrolidine-1-carboxylate (364a) (270 mg, 0.483 mmol) in dioxane/MeTHF (10 mL; ratio 9:1) using (1-aminoisoquinolin-5-yl)boronic acid (18a) (118 mg, 0.628 mmol), K₃PO₄ (2M aqueous solution, 410 mg, 1.934 mmol), PCy₃ (27.1 mg, 0.097 mmol), Pd₂(dba)₃ (44.3 mg, 0.048 mmol) and PdCl₂(dppf)-CH₂Cl₂Adduct (39.5 mg, 0.048 mmol) to afford after workup and purification using method-AE. (R)-isobutyl 3-(5-(1-aminoisoquinolin-5-yl)-3-((2-(2-ethoxy-2-oxoethyl)phenoxy)methyl)-1H-indazol-1-yl)pyrrolidine-1-carboxylate (365a) (147 mg, 49% yield) as a yellow solid; MS (ES+): 622.3 (M+1).

### Step-2: Preparation of (R)-2-(2-((5-(1-aminoisoquinolin-5-yl)-1-(1-(isobutoxycarbonyl)pyrrolidin-3-yl)-1H-indazol-3-yl)methoxy)phenyl)acetic acid (365b)

Compound 365b was prepared according to the procedure reported in step-2 of scheme-1, from (R)-isobutyl 3-(5-(1-aminoisoquinolin-5-yl)-3-((2-(2-ethoxy-2-oxoethyl)phenoxy)methyl)-1H-indazol-1-yl)pyrrolidine-1-carboxylate (365a) (143 mg, 0.230 mmol) in THF (5 mL) using lithium hydroxide hydrate (29 mg, 0.690 mmol) in water (1 mL) and stirring overnight at RT to afford after work up and purification using method-M, (R)-2-(2-((5-(1-aminoisoquinolin-5-yl)-1-(1-(isobutoxycarbonyl)pyrrolidin-3-yl)-1H-indazol-3-yl)methoxy)phenyl)acetic acid (365b) (34.4 mg, 25% yield) HCl salt as a white solid; ¹H NMR (300 MHz, DMSO-d₆) δ 13.17 (s, 1H, D₂O exchangeable), 12.00 (s, 1H, D₂O exchangeable), 9.07 (s, 2H, D₂O exchangeable), 8.61 (d, J=8.3 Hz, 1H), 7.99-7.82 (m, 4H), 7.63 (d, J=7.2 Hz, 1H), 7.52 (dd. J=8.7, 1.7 Hz, 1H), 7.28-7.21 (m, 2H), 7.21-7.14 (m, 1H), 6.99 (d, J=7.2 Hz, 1H), 6.94-6.87 (m, 1H), 5.63-5.51 (m, 1H), 5.45 (s, 2H), 3.98-3.88 (m, 1H), 3.88-3.80 (m, 2H), 3.78-3.64 (m, 2H), 3.61-3.51 (m, 1H), 3.48 (s, 2H), 2.48-2.34 (m, 2H), 1.98-1.80 (m, 1H), 0.97-0.84 (m, 6H); MS (ES+): 594.3 (M+1); (ES−): 592.2 (M−1); Optical Rotation: −8.0 [C 0.05, MeOH].

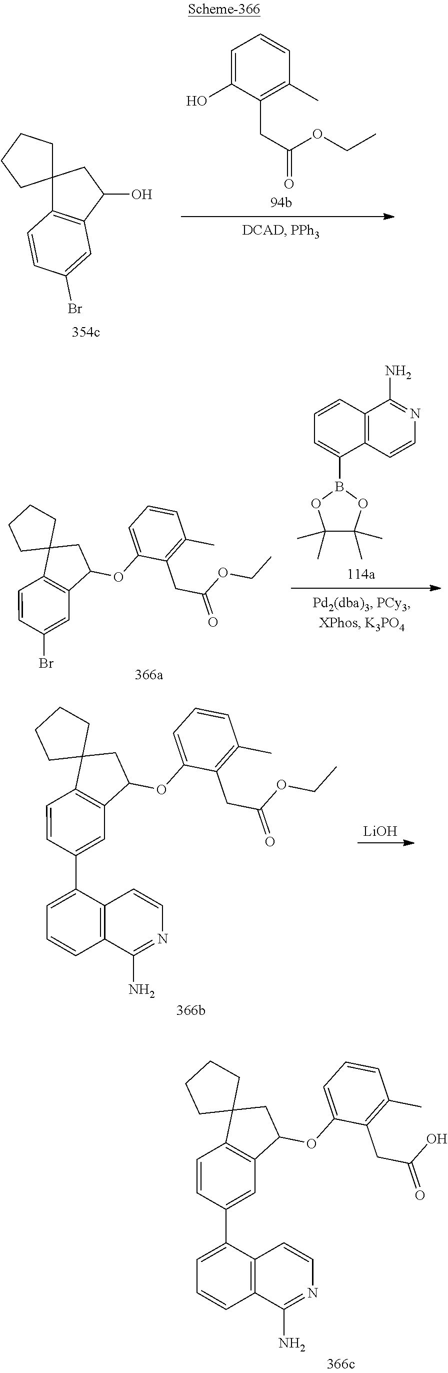

### Preparation of 2-(2-((5′-(1-aminoisoquinolin-5-yl)-2′,3′-dihydrospiro[cyclopentane-1,1′-inden]-3′-yl)oxy)-6-methylphenyl)acetic acid (366c)

### Step-1: Preparation of ethyl 2-(2-((5′-bromo-2′,3′-dihydrospiro[cyclopentane-1,1′-inden]-3′-yl)oxy)-6-methylphenyl)acetate (366a)

Compound 366a was prepared according to the procedure reported in step-2 of scheme-2, from 5′-bromo-2′,3′-dihydrospiro[cyclopentane-1,1′-inden]-3′-ol (354c) (1.1 g, 4.12 mmol) in DCM (20 mL) using PPh₃ (1.296 g, 4.94 mmol), ethyl 2-(2-hydroxy-6-methylphenyl)acetate (94b) 0.960 g, 4.94 mmol) and a solution of DCAD (1.814 g, 4.94 mmol) in DCM (60 mL) to afford after workup and purification using method-BS, ethyl 2-(2-((5′-bromo-2′,3′-dihydrospiro[cyclopentane-1,1′-inden]-3′-yl)oxy)-6-methylphenyl)acetate (366a) (750 mg, 41% yield) as clear oil; MS (ES+): 443.1 and 445.1 (M+1); 465.1 and 467.1 (M+Na); (ES−): 441.1 and 443.0 (M−1).

### Step-2: Preparation of ethyl 2-(2-((5′-(1-aminoisoquinolin-5-yl)-2′,3′-dihydrospiro[cyclopentane-1,1′-inden]-3′-yl)oxy)-6-methylphenyl)acetate (366b)

Compound 366b was prepared according to the procedure reported in step-3 of scheme-112, from ethyl 2-(2-((5′-bromo-2′,3′-dihydrospiro[cyclopentane-1,1′-inden]-3′-yl)oxy)-6-methylphenyl)acetate (366a) (150 mg, 0.338 mmol) in dioxane/2-MeTHF (2 mL) using 5-(4,4,5,5-tetramethyl-1,3,2-dioxaborolan-2-yl)isoquinolin-1-amine (114a) (183 mg, 0.677 mmol), K₃PO₄ (3 M aqueous solution, 226 μl, 0.677 mmol), PCy₃ (37.9 mg, 0.135 mmol), XPhos (32.3 mg, 0.068 mmol), Pd₂(dba)₃ (62.0 mg, 0.068 mmol) and heating at 100° C. for 10 h to afford after workup and purification using method-U, ethyl 2-(2-((5′-(1-aminoisoquinolin-5-yl)-2′,3′-dihydrospiro[cyclopentane-1,1′-inden]-3′-yl)oxy)-6-methylphenyl)acetate (366b) (88 mg, 51% yield) as a clear oil; MS (ES+): 507.3 (M+1).

### Step-3: Preparation of 2-(2-((5′-(1-aminoisoquinolin-5-yl)-2′,3′-dihydrospiro[cyclopentane-1,1′-inden]-3′-yl)oxy)-6-methylphenyl)acetic acid (366c)

Compound 366c was prepared according to the procedure reported in step-2 of scheme-1, from ethyl 2-(2-((5′-(1-aminoisoquinolin-5-yl)-2′,3′-dihydrospiro[cyclopentane-1,1′-inden]-3′-yl)oxy)-6-methylphenyl)acetate (366b) (88 mg, 0.174 mmol) in THF (3 mL) using lithium hydroxide (24 mg, 1.015 mmol) in water (1 mL) to afford after work up and purification using method-M, 2-(2-((5′-(1-aminoisoquinolin-5-yl)-2′,3′-dihydrospiro[cyclopentane-1,1′-inden]-3′-yl)oxy)-6-methylphenyl)acetic acid (366c) (26 mg, 16% yield) HCl salt as a white solid; ¹H NMR (300 MHz, DMSO-d₆) δ 13.26 (s, 1H, D₂O) exchangeable), 12.00 (s, 1H, D₂O exchangeable), 9.10 (s, 2H, D₂O exchangeable), 8.59 (d, J=8.2 Hz, 1H), 7.94-7.87 (m, 1H), 7.87-7.78 (m, 1H), 7.61 (d, J=7.3 Hz, 1H), 7.53-7.45 (m, 1H), 7.45-7.39 (m, 1H), 7.36 (d, J=1.6 Hz, 1H), 7.18-7.03 (m, 2H), 6.98 (d, J=7.2 Hz, 1H), 6.79 (d, J=7.2 Hz, 1H), 5.85 (t, J=5.7 Hz, 1H), 3.51 (s, 2H), 2.66-2.56 (m, 1H), 2.20 (s, 3H), 2.05-1.93 (m, 2H), 1.92-1.67 (m, 7H); MS (ES+): 479.3 (M+1); (ES−): 477.3 (M−1).

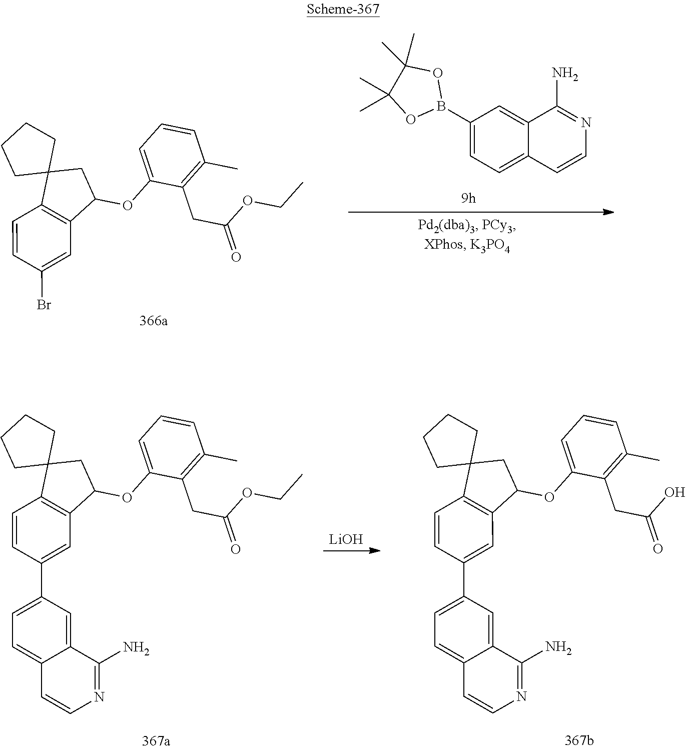 [SMILES: CCOC(=O)Cc1c(C)cccc1OC1CC2(CCCC2)c2ccc(Br)cc21 ; CC1(C)OB(c2ccc3ccnc(N)c3c2)OC1(C)C ; CCOC(=O)Cc1c(C)cccc1OC1CC2(CCCC2)c2ccc(-c3ccc4ccnc(N)c4c3)cc21 ; "LiOH" ; Cc1cccc(OC2CC3(CCCC3)c3ccc(-c4ccc5ccnc(N)c5c4)cc32)c1CC(=O)O]

### Preparation of 2-(2-((5′-(1-aminoisoquinolin-7-yl)-2′,3′-dihydrospiro[cyclopentane-1,1′-inden]-3′-yl)oxy)-6-methylphenyl)acetic acid (367b)

### Step-1: Preparation of ethyl 2-(2-((5′-(1-aminoisoquinolin-7-yl)-2′,3′-dihydrospiro[cyclopentane-1,1′-inden]-3′-yl)oxy)-6-methylphenyl)acetate (367a)

Compound 367a was prepared according to the procedure reported in step-3 of scheme-112, from ethyl 2-(2-((5′-bromo-2′,3′-dihydrospiro[cyclopentane-1,1′-inden]-3′-yl)oxy)-6-methylphenyl)acetate (366a) (150 mg, 0.338 mmol) in dioxane/2-MeTHF (2 mL) using 7-(4,4,5,5-tetramethyl-1,3,2-dioxaborolan-2-yl)isoquinolin-1-amine (9h) (183 mg, 0.677 mmol), K₃PO₄ (3M aqueous solution, 226 μl, 0.677 mmol), PCy₃ (38 mg, 0.135 mmol), XPhos (32 mg, 0.068 mmol), Pd₂(dba)₃ (62 mg, 0.068 mmol) and heating at 100° C. for 10 h to afford after workup and purification using method-U, ethyl 2-(2-((5′-(1-aminoisoquinolin-7-yl)-2′,3′-dihydrospiro[cyclopentane-1,1′-inden]-3′-yl)oxy)-6-methylphenyl)acetate (367a) (68 mg, 40% yield) as a clear oil; MS (ES+): 507.3 (M+1).

### Step-2: Preparation of 2-(2-((5′-(1-aminoisoquinolin-7-yl)-2′,3′-dihydrospiro[cyclopentane-1,1′-inden]-3′-yl)oxy)-6-methylphenyl)acetic acid (367b)

Compound 367b was prepared according to the procedure reported in step-2 of scheme-1, from ethyl 2-(2-((5′-(1-aminoisoquinolin-7-yl)-2′,3′-dihydrospiro[cyclopentane-1,1′-inden]-3′-yl)oxy)-6-methylphenyl)acetate (367a) (68 mg, 0.134 mmol) in THF (3 mL) using lithium hydroxide (24.30 mg, 1.015 mmol) in water (I mL) to afford after work up and purification using method-M, 2-(2-((5′-(1-aminoisoquinolin-7-yl)-2′,3′-dihydrospiro[cyclopentane-1,1′-inden]-3′-yl)oxy)-6-methylphenyl)acetic acid (367b) (31 mg, 19% yield) HCl salt as a white solid; ¹H NMR (300 MHz, DMSO-d₆) δ 13.26 (s, 1H, D₂O exchangeable), 12.13 (s, 1H, D₂O exchangeable), 9.08 (s, 2H, D₂O exchangeable), 8.88 (s, 1H), 8.29 (d, J=8.5 Hz, 1H), 8.03 (d, J=8.3 Hz, 1H), 7.89 (d, J=8.0 Hz, 1H), 7.84 (s, 1H), 7.73-7.67 (m, 1H), 7.49 (d, J=8.0 Hz, 1H), 7.26 (d, J=6.9 Hz, 1H), 7.22-7.06 (m, 2H), 6.82 (d, J=7.3 Hz, 1H), 5.86 (t, J=5.7 Hz, 1H), 3.61-3.46 (m, 2H), 2.62-2.52 (m, 1H), 2.21 (s, 3H), 2.03-1.91 (m, 2H), 1.89-1.60 (m, 7H); MS (ES+): 479.2 (M+1); (ES−): 477.2 (M−1).

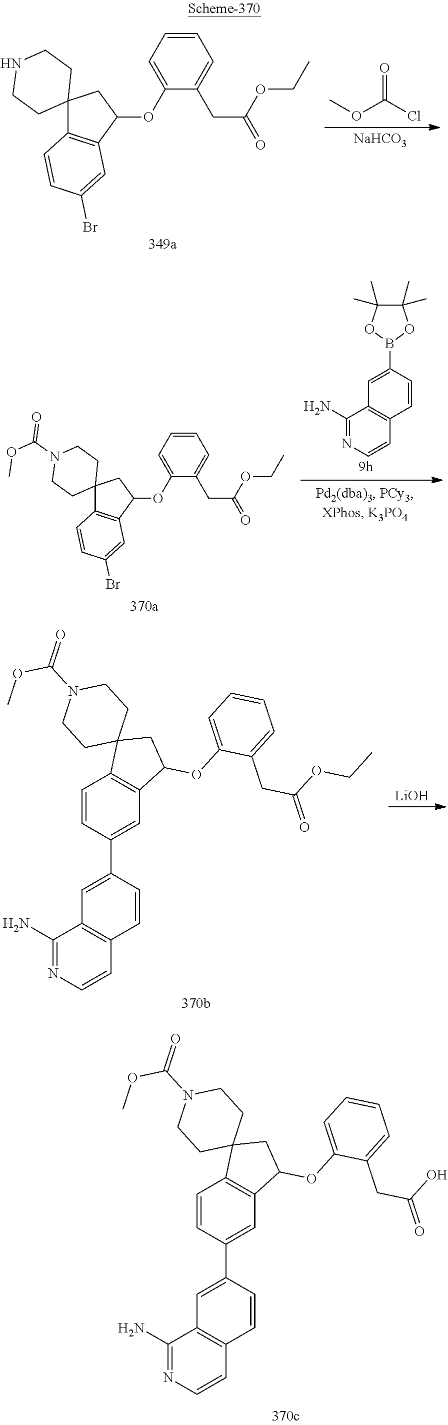

### Preparation of 2-(2-((5-(1-aminoisoquinolin-7-yl)-1′-(methoxycarbonyl)-2,3-dihydrospiro[indene-1,4′-piperidin]-3-yl)oxy)phenyl)acetic acid (370c)

### Step-1: Preparation of methyl 5-bromo-3-(2-(2-ethoxy-2-oxoethyl)phenoxy)-2,3-dihydrospiro[indene-1,4′-piperidine]-1′-carboxylate (370a)

Compound 370a was prepared according to the procedure reported in step-1 of scheme-126, from ethyl 2-(2-((5-bromo-2,3-dihydrospiro[indene-1,4′-piperidin]-3-yl)oxy)phenyl)acetate, TFA (349a) (0.56 g, 1.003 mmol) in THF (10 mL) using sodium bicarbonate (2.507 mL, 5.01 mmol), methyl carbonochloridate (0.116 mL, 1.504 mmol) and stirring at RT for 12 h to afford after workup and purification using method-P, methyl 5-bromo-3-(2-(2-ethoxy-2-oxoethyl)phenoxy)-2,3-dihydrospiro[indene-1,4′-piperidine]-1′-carboxylate (370a) (0.31 g, 62% yield) as a clear oil; MS (ES+): 524.1 (M+Na).

### Step-2: Preparation of methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (370b)

Compound 370b was prepared according to the procedure reported in step-3 of scheme-112, from methyl 5-bromo-3-(2-(2-ethoxy-2-oxoethyl)phenoxy)-2,3-dihydrospiro[indene-1,4′-piperidine]-1′-carboxylate (370a) (150 mg, 0.299 mmol) in dioxane/2-MeTHF (2 mL) using 7-(4,4,5,5-tetramethyl-1,3,2-dioxaborolan-2-yl)isoquinolin-1-amine (9h) (161 mg, 0.597 mmol), K₃PO₄ (3M aqueous solution, 199 μl, 0.597 mmol), PCy₃ (33.5 mg, 0.119 mmol), XPhos (14.23 mg, 0.03 mmol), Pd₂(dba)₃ (54.7 mg, 0.06 mmol) and heating at 100° C. for 10 h to afford after workup and purification using method-M, methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (370b) (70 mg, 41% yield) as a white solid; MS (ES+): 566.3 (M+1); (ES−): 564.3 (M−1).

### Step-3: Preparation of 2-(2-((5-(1-aminoisoquinolin-7-yl)-1′-(methoxycarbonyl)-2,3-dihydrospiro[indene-1,4′-piperidin]-3-yl)oxy)phenyl)acetic acid (370c)

Compound 370c was prepared according to the procedure reported in step-2 of scheme-1, from methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (370b) (45 mg, 0.08 mmol) in MeOH (1 mL) and THF (I mL) using lithium hydroxide (1N solution, 239 μl, 0.239 mmol) to afford after work up and purification using method-M, 2-(2-((5-(1-aminoisoquinolin-7-yl)-1′-(methoxycarbonyl)-2,3-dihydrospiro[indene-1,4′-piperidin]-3-yl)oxy)phenyl)acetic acid (370c) (28 mg, 66% yield) HCl salt as a white solid; ¹H NMR (300 MHz, DMSO-d₆) δ 13.17 (s, 1H, D₂O exchangeable), 12.09 (s, 1H, D₂O exchangeable), 9.05 (s, 2H, DO exchangeable), 8.88 (s, 1H), 8.31 (dd, J=8.5, 1.6 Hz, 1H), 8.03 (d, J=8.5 Hz, 1H), 7.95-7.83 (m, 2H), 7.69 (d, J=6.9 Hz, 1H), 7.57 (d, J=8.0 Hz, 1H), 7.37-7.18 (m, 4H), 6.97-6.88 (m, 1H), 5.95 (dd, J=6.8, 4.1 Hz, 1H), 4.17-3.89 (m, 2H), 3.63 (s, 3H), 3.47 (d, J=3.6 Hz, 2H), 3.19-2.80 (m, 2H), 2.74 (dd, J=13.7, 6.8 Hz, 1H), 2.08 (dd, J=13.7, 4.1 Hz, 1H), 1.97-1.74 (m, 2H), 1.67-1.50 (m, 2H); MS (ES+): 538.2 (M+1); (ES−): 536.2 (M−1).

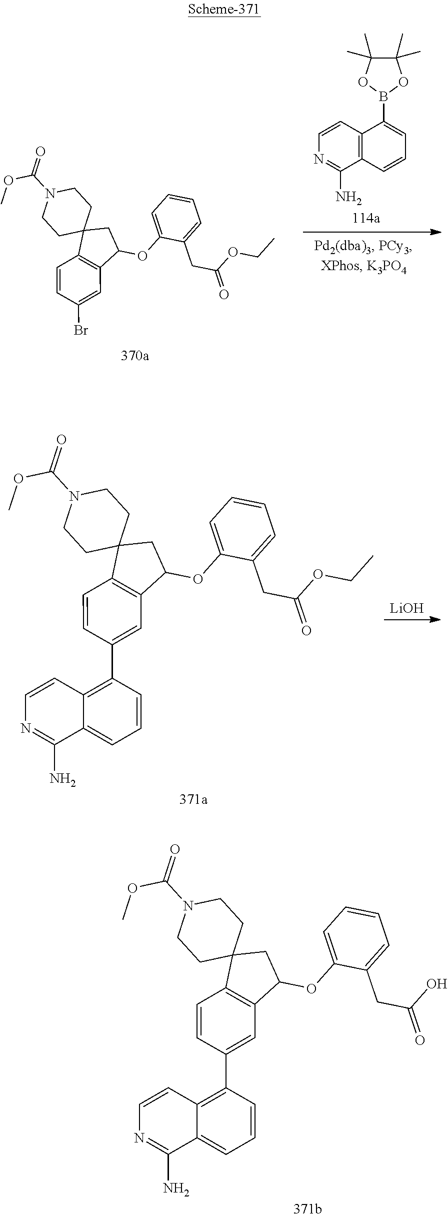

### Preparation of 2-(2-((5-(1-aminoisoquinolin-5-yl)-1′-(methoxycarbonyl)-2,3-dihydrospiro[indene-1,4′-piperidin]-3-yl)oxy)phenyl)acetic acid (371b)

### Step-1: Preparation of methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (371a)

Compound 371a was prepared according to the procedure reported in step-3 of scheme-112, from methyl 5-bromo-3-(2-(2-ethoxy-2-oxoethyl)phenoxy)-2,3-dihydrospiro[indene-1,4′-piperidine]-1′-carboxylate (370a) (150 mg, 0.299 mmol) in dioxane/2-MeTHF (2 mL) using 5-(4,4,5,5-tetramethyl-1,3,2-dioxaborolan-2-yl)isoquinolin-1-amine (114a) (161 mg, 0.597 mmol), K₃PO₄ (3M aqueous solution, 199 μl, 0.597 mmol), PCy₃ (33.5 mg, 0.119 mmol), XPhos (14.23 mg, 0.030 mmol), Pd₂(dba)₃ (54.7 mg, 0.06 mmol) and heating at 100° C. for 10 h to afford after workup and purification using method-M, methyl 5-(1-aminoisoquinolin-S-yl)-3-(2-(2-ethoxy-2-oxoethyl)phenoxy)-2,3-dihydrospiro[indene-1,4′-piperidine]-1′-carboxylate (371a) (72 mg, 42.6% yield) as a pale yellow solid; MS (ES+): 566.3 (M+1).

### Step-3: Preparation of 2-(2-((5-(1-aminoisoquinolin-5-yl)-1′-(methoxycarbonyl)-2,3-dihydrospiro[indene-1,4′-piperidin]-3-yl)oxy)phenyl)acetic acid (371b)

Compound 371b was prepared according to the procedure reported in step-2 of scheme-1, from methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (371a) (50 mg, 0.088 mmol) in MeOH (1 ml) and THF (1 mL) using lithium hydroxide (1 N solution, 265 μl, 0.265 mmol) to afford after work up and purification using method-M, 2-(2-((5-(1-aminoisoquinolin-5-yl)-1′-(methoxycarbonyl)-2,3-dihydrospiro[indene-1,4′-piperidin]-3-yl)oxy)phenyl)acetic acid (371b) (37 mg, 78% yield) HCl salt as a white solid; ¹H NMR (300 MHz, DMSO-d₆) δ 13.32 (s, 1H, D₂O exchangeable), 12.03 (s, 1H, D₂O exchangeable), 9.12 (s, 2H, D₂O exchangeable), 8.60 (d, J=8.2 Hz, 1H), 7.95-7.78 (m, 2H), 7.61 (d, J=7.2 Hz, 1H), 7.55 (d, J=7.8 Hz, 1H), 7.47-7.38 (m, 2H), 7.34-7.22 (m, 2H), 7.22-7.16 (m, 1H), 6.96 (d, J=7.2 Hz, 1H), 6.94-6.86 (m, 1H), 5.94 (dd, J=6.8, 4.5 Hz, 1H), 4.04 (s, 2H), 3.63 (s, 3H), 3.44 (d, J=1.5 Hz, 2H), 3.20-2.87 (m, 2H), 2.80 (dd, J=13.7, 6.9 Hz, 1H), 2.09 (dd, J=13.7, 4.5 Hz, 1H), 2.03-1.73 (m, 2H), 1.73-1.49 (m, 2H); MS (ES+): 538.3 (M+1); (ES−): 536.3 (M−1).

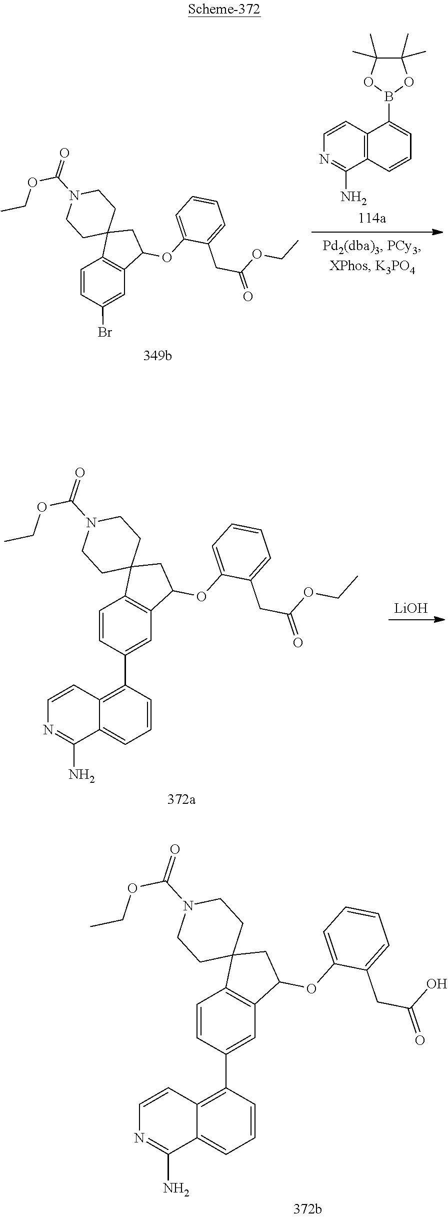

### Preparation of 2-(2-((5-(1-aminoisoquinolin-5-yl)-1′-(ethoxycarbonyl)-2,3-dihydrospiro[indene-1,4′-piperidin]-3-yl)oxy)phenyl)acetic acid (372b)

### Step-1: Preparation of ethyl 5-(1-aminoisoquinolin-5-yl)-3-(2-(2-ethoxy-2-oxoethyl)phenoxy)-2,3-dihydrospiro[indene-1,4′-piperidine]-1′-carboxylate (372a)

Compound 372a was prepared according to the procedure reported in step-3 of scheme-112, from ethyl 5-bromo-3-(2-(2-ethoxy-2-oxoethyl)phenoxy)-2,3-dihydrospiro[indene-1,4′-piperidine]-1′-carboxylate (349b) (155 mg, 0.300 mmol) in dioxane/2-MeTHF (2 mL) using 5-(4,4,5,5-tetramethyl-1,3,2-dioxaborolan-2-yl)isoquinolin-1-amine (114a) (162 mg, 0.6 mmol), K₃PO₄ (3 M aqueous solution, 200 μl, 0.600 mmol), PCy₃ (33.7 mg, 0.120 mmol), XPhos (14 mg, 0.030 mmol), Pd₂(dba)₃ (55 mg, 0.06 mmol) and heating at 100° C. for 10 h to afford after workup and purification using method-M, ethyl 5-(1-aminoisoquinolin-5-yl)-3-(2-(2-ethoxy-2-oxoethyl)phenoxy)-2,3-dihydrospiro[indene-1,4′-piperidine]-1′-carboxylate (372a) (35 mg, 20% yield) as a pale yellow solid; MS (ES+): 580.3 (M+1).

### Step-2: Preparation of 2-(2-((5-(1-aminoisoquinolin-5-yl)-1′-(ethoxycarbonyl)-2,3-dihydrospiro[indene-1,4′-piperidin]-3-yl)oxy)phenyl)acetic acid (372b)

Compound 372b was prepared according to the procedure reported in step-2 of scheme-1, from ethyl 5-(1-aminoisoquinolin-5-yl)-3-(2-(2-ethoxy-2-oxoethyl)phenoxy)-2,3-dihydrospiro[indene-1,4′-piperidine]-1′-carboxylate (372a) (35 mg, 0.06 mmol) in MeOH (1 ml) and THF (1 mL) using lithium hydroxide (1N solution, 181 μl, 0.181 mmol) to afford after work up and purification using method-M, 2-(2-((5-(1-aminoisoquinolin-5-yl)-1′-(ethoxycarbonyl)-2,3-dihydrospiro[indene-1,4′-piperidin]-3-yl)oxy)phenyl)acetic acid (372b) (12 mg, 36% yield) HCl salt as a pale yellow solid; ¹H NMR (300 MHz, DMSO-d₆) δ 13.15 (s, 1H, D₂O exchangeable), 12.02 (s, 1H, D₂O) exchangeable), 9.08 (s, 2H, D₂O exchangeable), 8.59 (d, J=8.2 Hz, 1H), 7.95-7.78 (m, 2H), 7.61 (d, J=7.3 Hz, 1H), 7.56 (d, J=7.8 Hz, 1H), 7.49-7.38 (m, 2H), 7.34-7.23 (m, 2H), 7.23-7.15 (m, 1H), 6.96 (d, J=7.3 Hz, 1H), 6.94-6.86 (m, 1H), 5.94 (t, J=5.6 Hz, 1H), 4.16-3.96 (m, 4H), 3.44 (s, 2H), 3.19-3.01 (m, 1H), 3.01-2.86 (m, 1H), 2.80 (dd, J=13.7, 6.9 Hz, 1H), 2.09 (dd, J=13.6, 4.5 Hz, 1H), 2.00-1.87 (m, 1H), 1.87-1.73 (m, 1H), 1.67 (d, J=12.8 Hz, 1H), 1.58 (d, J=12.8 Hz, 1H), 1.21 (t, J=7.1 Hz, 3H). MS (ES+): 552.2 (M+1); (ES−): 550.3 (M−1).

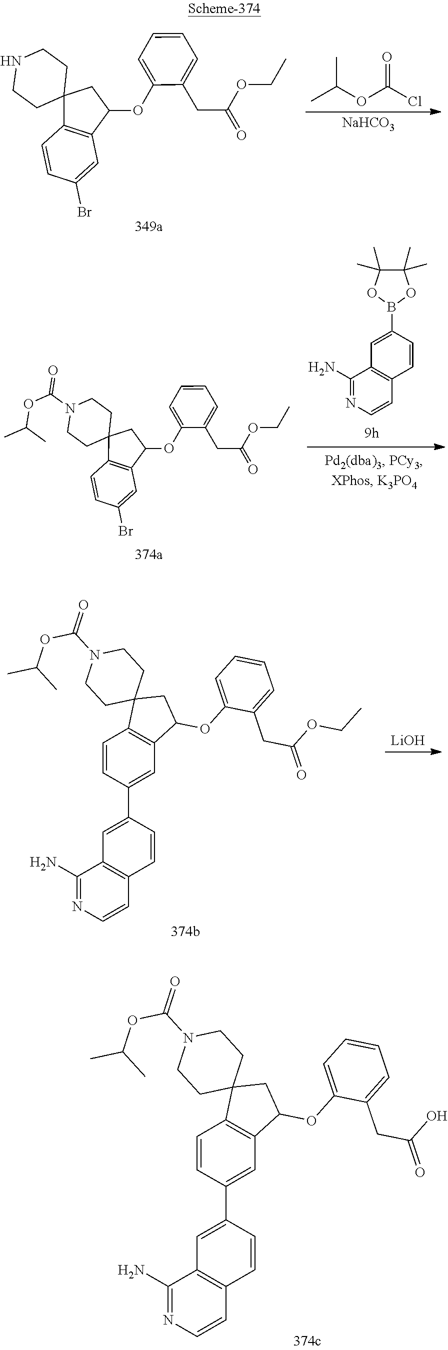

### Preparation of 2-(2-((5-(1-aminoisoquinolin-7-yl)-1′-(isopropoxycarbonyl)-2,3-dihydrospiro[indene-1,4′-piperidin]-3-yl)oxy)phenyl)acetic acid (374c)

### Step-1: Preparation of isopropyl 5-bromo-3-(2-(2-ethoxy-2-oxoethyl)phenoxy)-2,3-dihydrospiro[indene-1,4′-piperidine]-1′-carboxylate (374a)

Compound 374a was prepared according to the procedure reported in step-1 of scheme-126, from a solution of ethyl 2-(2-((5-bromo-2,3-dihydrospiro[indene-1,4′-piperidin]-3-yl)oxy)phenyl)acetate, TFA (349a) (560 mg, 1.003 mmol) in THF (10 mL) using sodium bicarbonate (2507 μl, 5.01 mmol) and isopropyl carbonochloridate (1504 μl, 1.504 mmol) to afford after workup and purification using method-P, isopropyl 5-bromo-3-(2-(2-ethoxy-2-oxoethyl)phenoxy)-2,3-dihydrospiro[indene-1,4′-piperidine]-1′-carboxylate (374a) (0.395 g, 74% yield) as a clear oil; MS (ES+): 552.2 (M+Na).

### Step-2: Preparation of isopropyl 5-(1-aminoisoquinolin-7-yl)-3-(2-(2-ethoxy-2-oxoethyl)phenoxy)-2,3-dihydrospiro[indene-1,4′-piperidine]-1′-carboxylate (374b)

Compound 374b was prepared according to the procedure reported in step-3 of scheme-112, from isopropyl 5-bromo-3-(2-(2-ethoxy-2-oxoethyl)phenoxy)-2,3-dihydrospiro[indene-1,4′-piperidine]-1′-carboxylate (374a) (191 mg, 0.36 mmol) in dioxane/2-MeTHF (2 mL) using 7-(4,4,5,5-tetramethyl-1,3,2-dioxaborolan-2-yl)isoquinolin-1-amine (9h) (195 mg, 0.72 mmol), K₃PO₄ (3 M aqueous solution, 240 μl, 0.72 mmol), PCy₃ (40 mg, 0.144 mmol), XPhos (17 mg, 0.036 mmol), Pd₂(dba)₃ (65.9 mg, 0.072 mmol) and heating at 100° C. for 10 h to afford after workup and purification using method-M, isopropyl 5-(1-aminoisoquinolin-7-yl)-3-(2-(2-ethoxy-2-oxoethyl)phenoxy)-2,3-dihydrospiro[indene-1,4′-piperidine]-1′-carboxylate (374b) (71 mg, 33% yield) as a white solid; MS (ES+): 594.3 (M+1).

### Step-3: Preparation of 2-(2-((5-(1-aminoisoquinolin-7-yl)-1′-(isopropoxycarbonyl)-2,3-dihydrospiro[indene-1,4′-piperidin]-3-yl)oxy)phenyl)acetic acid (374c)

Compound 374c was prepared according to the procedure reported in step-2 of scheme-1, from isopropyl 5-(1-aminoisoquinolin-7-yl)-3-(2-(2-ethoxy-2-oxoethyl)phenoxy)-2,3-dihydrospiro[indene-1,4′-piperidine]-1′-carboxylate (374b) (50 mg, 0.084 mmol) in MeOH (1 ml) and THF (1 mL) using lithium hydroxide (1N solution, 253 μl, 0.253 mmol) to afford after work up and purification using method-M, 2-(2-((5-(1-aminoisoquinolin-7-yl)-1′-(isopropoxycarbonyl)-2,3-dihydrospiro[indene-1,4′-piperidin]-3-yl)oxy)phenyl)acetic acid (374c) (31 mg, 65% yield) HCl salt as a white solid; ¹H NMR (300 MHz, DMSO-d₆) δ 13.21 (s, 1H, D₂O exchangeable), 12.12 (s, 1H, D₂O exchangeable), 9.03 (s, 2H, D₂O exchangeable), 8.87 (d, J=1.8 Hz, 1H), 8.30 (dd, J=8.5, 1.6 Hz, 1H), 8.03 (d, J=8.5 Hz, 1H), 7.96-7.81 (m, 2H), 7.69 (d, J=6.9 Hz, 1H), 7.57 (d, J=8.0 Hz, 1H), 7.38-7.15 (m, 4H), 6.98-6.86 (m, 1H), 5.94 (dd, J=6.9, 4.1 Hz, 1H), 4.88-4.71 (m, 1H), 4.13-3.93 (m, 2H), 3.47 (d, J=3.5 Hz, 2H), 3.17-2.79 (m, 2H), 2.74 (dd, J=13.9, 6.9 Hz, 1H), 2.07 (dd, J=13.8, 4.2 Hz, 1H), 1.96-1.70 (m, 2H), 1.70-1.45 (m, 2H), 1.21 (d, J=6.2 Hz, 6H); MS (ES+): 566.3 (M+1); (ES−): 564.3 (M−1).

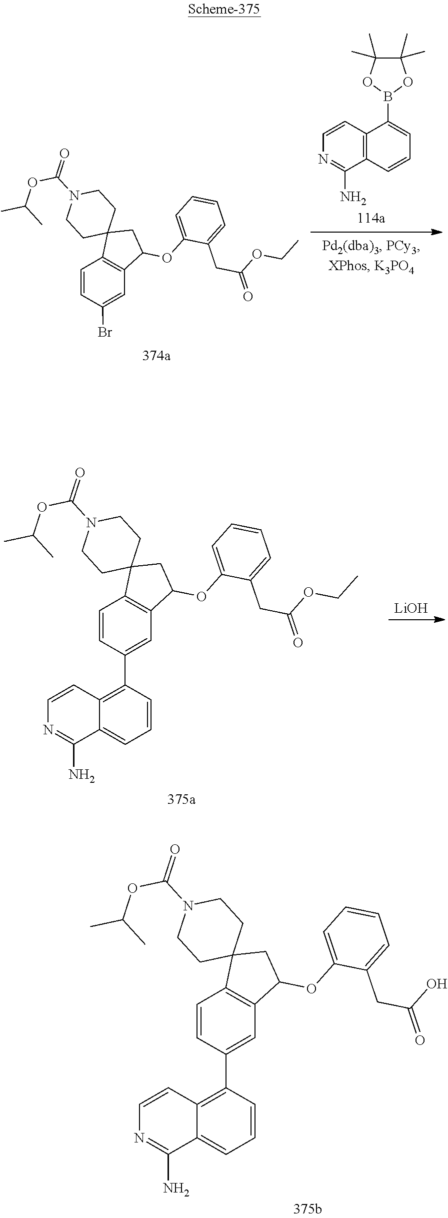

### Preparation of 2-(2-((5-(1-aminoisoquinolin-5-yl)-1′-(isopropoxycarbonyl)-2,3-dihydrospiro[indene-1,4′-piperidin]-3-yl)oxy)phenyl)acetic acid (375b)

### Step-1: Preparation of isopropyl 5-(1-aminoisoquinolin-5-yl)-3-(2-(2-ethoxy-2-oxoethyl)phenoxy)-2,3-dihydrospiro[indene-1,4′-piperidine]-1′-carboxylate (375a)

Compound 375a was prepared according to the procedure reported in step-3 of scheme-112, from isopropyl 5-bromo-3-(2-(2-ethoxy-2-oxoethyl)phenoxy)-2,3-dihydrospiro[indene-1,4′-piperidine]-1′-carboxylate (374a) (191 mg, 0.36 mmol) in dioxane/2-MeTHF (2 mL) using 5-(4,4,5,5-tetramethyl-1,3,2-dioxaborolan-2-yl)isoquinolin-1-amine (114a) (195 mg, 0.72 mmol), K₃PO₄ (3M aqueous solution, 240 μl, 0.72 mmol), PCy₃ (40 mg, 0.144 mmol), XPhos (17 mg, 0.036 mmol), Pd₂(dba)₃ (66 mg, 0.072 mmol) and heating at 100° C. for 10 h to afford after workup and purification using method-M, isopropyl 5-(1-aminoisoquinolin-5-yl)-3-(2-(2-ethoxy-2-oxoethyl)phenoxy)-2,3-dihydrospiro[indene-1,4′-piperidine]-1′-carboxylate (375a) (92 mg, 43.0% yield) as a white solid; MS (ES+): 594.3 (M+1).

### Step-2: Preparation of 2-(2-((5-(1-aminoisoquinolin-5-yl)-1′-(isopropoxycarbonyl)-2,3-dihydrospiro[indene-1,4′-piperidin]-3-yl)oxy)phenyl)acetic acid (375b)

Compound 375b was prepared according to the procedure reported in step-2 of scheme-1, from isopropyl 5-(1-aminoisoquinolin-5-yl)-3-(2-(2-ethoxy-2-oxoethyl)phenoxy)-2,3-dihydrospiro[indene-1,4′-piperidine]-1′-carboxylate (375a) (70 mg, 0.118 mmol) in MeOH (1 ml) and THF (1 mL) using lithium hydroxide (1N solution, 354 μl, 0.354 mmol) to afford after work up and purification using method-M, 2-(2-((5-(1-aminoisoquinolin-5-yl)-1′-(isopropoxycarbonyl)-2,3-dihydrospiro[indene-1,4′-piperidin]-3-yl)oxy)phenyl)acetic acid (375b) (53 mg, 79% yield) HCl salt as a white solid; ¹H NMR (300 MHz, DMSO-d₆) δ 13.32 (s, 1H, D₂O exchangeable), 12.04 (s, 1H, D₂O exchangeable), 9.11 (s, 2H, D₂O exchangeable), 8.60 (d, J=8.2 Hz, 1H), 7.95-7.78 (m, 2H), 7.62 (d, J=7.2 Hz, 1H), 7.55 (d, J=7.8 Hz, 1H), 7.47-7.37 (m, 2H), 7.31-7.22 (m, 2H), 7.22-7.16 (m, 1H), 7.00-6.86 (m, 2H), 5.94 (dd, J=6.8, 4.5 Hz, 1H), 4.91-4.71 (m, 1H), 4.21-3.80 (m, 2H), 3.44 (d, J=1.5 Hz, 2H), 3.19-2.86 (m, 2H), 2.80 (dd, J=13.7, 6.9 Hz, 1H), 2.16-2.03 (m, 1H), 1.99-1.86 (m, 1H), 1.86-1.72 (m, 1H), 1.73-1.52 (m, 2H), 1.22 (d, J=6.2 Hz, 6H). MS (ES+): 566.3 (M+1); (ES−): 564.3 (M−1).

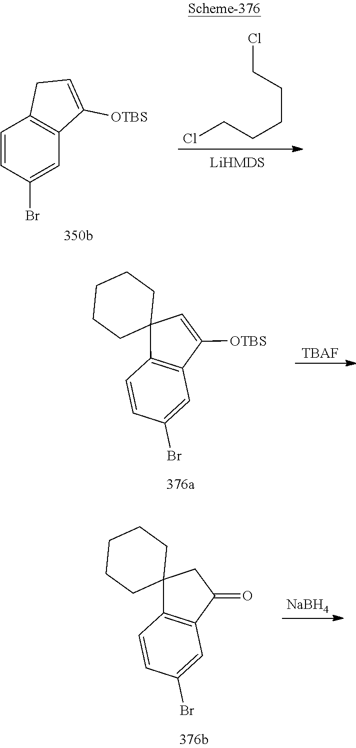

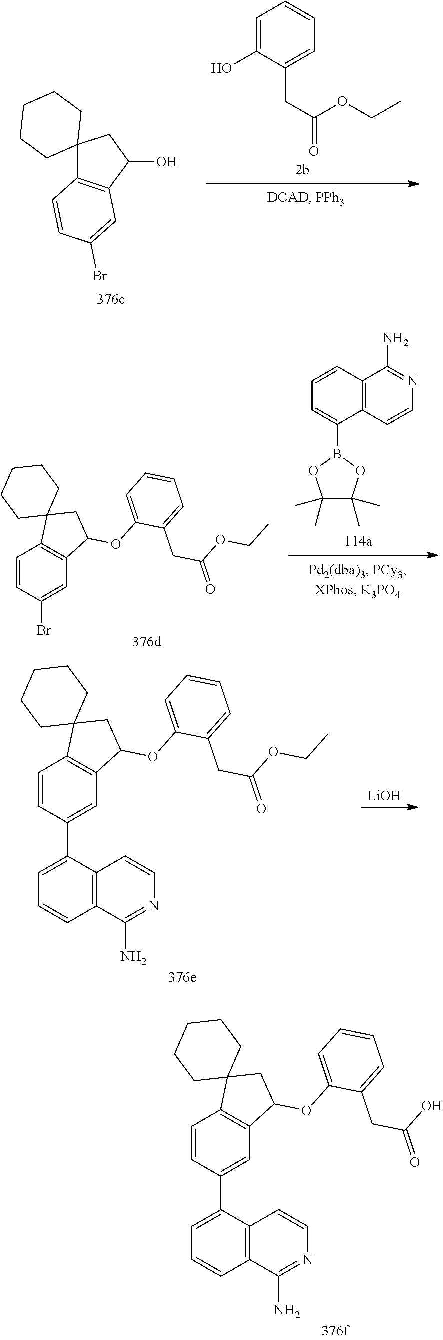

### Preparation of 2-(2-((5′-(1-aminoisoquinolin-5-yl)-2′,3′-dihydrospiro[cyclohexane-1,1′-inden]-3′-yl)oxy)phenyl)acetic acid (376f)

### Step-1: Preparation of ((5′-bromospiro[cyclohexane-1,1′-inden]-3′-yl)oxy)(tert-butyl)dimethylsilane (376a)

Compound 376a was prepared according to the procedure reported in step-2 of scheme-350, from ((5-bromo-1H-inden-3-yl)oxy)(tert-butyl)dimethylsilane (350b) (4 g, 12.30 mmol) in anhydrous THF (30 mL) using LiHMDS (1.0 M in THF, 30.7 mL, 30.7 mmol) and a solution of 1,5-dichloropentane (2.081 g, 14.75 mmol) in anhydrous THF (2 mL) to afford after work up and purification using method-I, to afford ((5′-bromospiro[cyclohexane-1,1′-inden]-3′-yl)oxy)(tert-butyl)dimethylsilane (376a) (820 mg, 17% yield) as a colorless oil, which became a white solid after standing at RT; ¹H NMR (300 MHz, DMSO-d₆) δ 7.37 (d, J=1.2 Hz, 2H), 7.25 (t, J=1.2 Hz, 1H), 5.92 (s, 1H), 1.89-1.69 (m, 5H), 1.62-1.32 (m, 3H), 1.22-1.11 (m, 2H), 0.97 (s, 9H), 0.24 (s, 6H).

### Step-2: Preparation of 5′-bromospiro[cyclohexane-1,1′-inden]-3′(2′H)-one (376b)

Compound 376b was prepared according to the procedure reported in step-3 of scheme-350, from ((5′-bromospiro[cyclohexane-1,1′-inden]-3′-yl)oxy)(tert-butyl)dimethylsilane (376a) (6 g, 15.25 mmol) in anhydrous THF (50 mL) using TBAF (22.88 mL, 22.88 mmol) to afford after work up and purification using method-I, to afford 5′-bromospiro[cyclohexane-1,1′-inden]-3′(2′H)-one (376b) (2.2 g, 51% yield) as a yellow solid; ¹H NMR (300 MHz, DMSO-d₆) δ 7.85 (dd. J=8.2, 2.0 Hz, 1H), 7.72-7.65 (m, 2H), 2.60 (s, 2H), 1.80-1.64 (m, 5H), 1.50-1.26 (m, 5H).

### Step-3: Preparation of 5′-bromo-2′,3′-dihydrospiro[cyclohexane-1,1′inden]-3′-ol (376c)

Compound 376c was prepared according to the procedure reported in step-1 of scheme-205, from 5′-bromospiro[cyclohexane-1,1′-inden]-3′(2′H)-one (376b) (2.2 g, 7.88 mmol) in anhydrous MeOH (20 mL) using sodium borohydride (0.447 g, 11.82 mmol) to afford after work up and purification using method-J, 5′-bromo-2′,3′-dihydrospiro[cyclohexane-1,1′-inden]-3′-ol (376c) (1.9 g, 86% yield) as a yellow oil; ¹H NMR (300 MHz, DMSO-d₆) δ 7.49-7.39 (m, 2H), 7.22 (d, J=8.0 Hz, 1H), 5.35 (d, J=5.7 Hz, 1H), 5.08 (q, J=6.5 Hz, 1H), 2.51-2.41 (m, 1H), 1.77-1.62 (m, 5H), 1.60-1.49 (m, 2H), 1.45-1.37 (m, 3H), 1.33-1.25 (m, 1H).

### Step-4: Preparation of ethyl 2-(2-((5′-bromo-2′,3′-dihydrospiro[cyclohexane-1,1′-inden]-3′-yl)oxy)phenyl)acetate (376d)

Compound 376d was prepared according to the procedure reported in step-2 of scheme-2, from 5′-bromo-2′,3′-dihydrospiro[cyclohexane-1,1′-inden]-3′-ol (376c) (950 mg, 3.38 mmol) in DCM (20 mL) using PPh₃ (1063 mg, 4.05 mmol), ethyl 2-(2-hydroxyphenyl)acetate (2b) (731 mg, 4.05 mmol) and a solution of DCAD (1489 mg, 4.05 mmol) in DCM (60 mL) to afford after workup and purification using method-BS, ethyl 2-(2-((5′-bromo-2′,3′-dihydrospiro[cyclohexane-1,1′-inden]-3′-yl)oxy)phenyl)acetate (376d) (428 mg, 28.6% yield) as a clear oil; MS (ES+): 443.1 and 445.1 (M+1), 465.1 and 467.1 (M+Na).

### Step-5: Preparation of ethyl 2-(2-((5′-(1-aminoisoquinolin-5-yl)-2′,3′-dihydrospiro[cyclohexane-1,1′-inden]-3′-yl)oxy)phenyl)acetate (376e)

Compound 376e was prepared according to the procedure reported in step-3 of scheme-112, from ethyl 2-(2-((5′-bromo-2′,3′-dihydrospiro[cyclohexane-1,1′-inden]-3′-yl)oxy)phenyl)acetate (376d) (120 mg, 0.271 mmol) in dioxane/2-MeTHF (2 mL) using 5-(4,4,5,5-tetramethyl-1,3,2-dioxaborolan-2-yl)isoquinolin-1-amine (114a) (146 mg, 0.541 mmol), K₃PO₄ (3M aqueous solution, 180 μl, 0.541 mmol). PCy₃ (30.4 mg, 0.108 mmol). XPhos (25.8 mg, 0.054 mmol) and Pd₂(dba)₃ (49.6 mg, 0.054 mmol) and heating at 100° C. for 10 h to afford after workup and purification using method-U, ethyl 2-(2-((5′-(1-aminoisoquinolin-5-yl)-2′,3′-dihydrospiro[cyclohexane-1,1′-inden]-3′-yl)oxy)phenyl)acetate (376e) (96 mg, 70% yield) as a clear oil; MS (ES+): 507.3 (M+1); (ES−): 505.2 (M−1).

### Step-6: Preparation of 2-(2-((5′-(1-aminoisoquinolin-5-yl)-2′,3′-dihydrospiro[cyclohexane-1,1′-inden]-3′-yl)oxy)phenyl)acetic acid (376f)

Compound 376f was prepared according to the procedure reported in step-2 of scheme-1, from ethyl 2-(2-((5′-(1-aminoisoquinolin-5-yl)-2′,3′-dihydrospiro[cyclohexane-1,1′-inden]-3′-yl)oxy)phenyl)acetate (376e) (96 mg, 0.189 mmol) in THF (3 mL) using lithium hydroxide (19.44 mg, 0.812 mmol) in water (1 mL) to afford after work up and purification using method-M, 2-(2-((5′-(1-aminoisoquinolin-5-yl)-2′,3′-dihydrospiro[cyclohexane-1,1′-inden]-3′-yl)oxy)phenyl)acetic acid (376f) (67 mg, 52% yield) HCl salt as a white solid; ¹H NMR (300 MHz, DMSO-d₆) δ 13.29 (s, 1H, D₂O exchangeable), 12.03 (s, 1H, D₂O exchangeable), 9.12 (s, 2H, D₂O exchangeable), 8.60 (d, J=8.2 Hz, 1H), 7.95-7.88 (m, 1H), 7.83 (t, J=7.8 Hz, 1H), 7.65-7.58 (m, 1H), 7.55-7.47 (m, 1H), 7.46-7.35 (m, 2H), 7.32-7.23 (m, 2H), 7.23-7.16 (m, 1H), 6.98 (d, J=7.2 Hz, 1H), 6.95-6.86 (m, 1H), 5.94-5.80 (m, 1H), 3.45 (d, J=1.4 Hz, 2H), 2.78-2.66 (m, 1H), 1.98 (dd, J=13.6, 4.8 Hz, 1H), 1.85-1.52 (m, 8H), 1.46-1.24 (m, 2H); MS (ES+): 479.2 (M+1); (ES−): 477.3 (M−1).

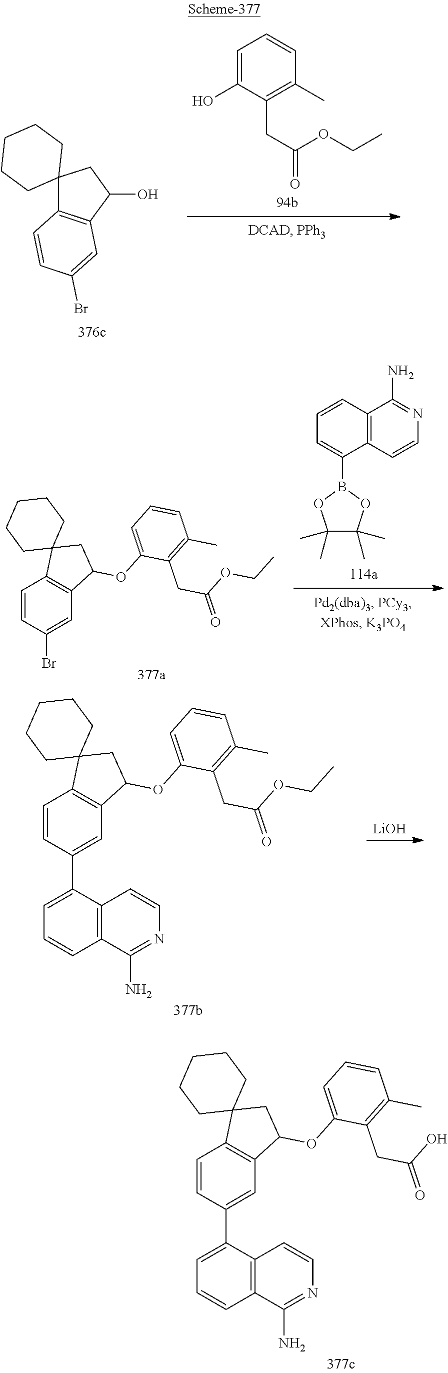

### Preparation of 2-(2-((5′-(1-aminoisoquinolin-5-yl)-2′,3′-dihydrospiro[cyclohexane-1,1′-inden]-3′-yl)oxy)-6-methylphenyl)acetic acid (377c)

### Step-1: Preparation of ethyl 2-(2-((5′-bromo-2′,3′-dihydrospiro[cyclohexane-1,1′-inden]-3′-yl)oxy)-6-methylphenyl)acetate (377a)

Compound 377a was prepared according to the procedure reported in step-2 of scheme-2, from 5′-bromo-2′,3′-dihydrospiro[cyclohexane-1,1′-inden]-3′-ol (376c) (950 mg, 3.38 mmol) in DCM (20 mL) using PPh₃ (1063 mg, 4.05 mmol), ethyl 2-(2-hydroxy-6-methylphenyl)acetate (94b) (787 mg, 4.05 mmol) and a solution of DCAD (1489 mg, 4.05 mmol) in DCM (60 mL) to afford after workup and purification using method-BS, ethyl 2-(2-((5′-bromo-2′,3′-dihydrospiro[cyclohexane-1,1′-inden]-3′-yl)oxy)-6-methylphenyl)acetate (377a) (300 mg, 19% yield) as a clear oil; MS (ES+): 479.1 and 481.1 (M+Na); (ES−): 455.0 and 457.1 (M−1).

### Step-2: Preparation of ethyl 2-(2-((5′-(1-aminoisoquinolin-5-yl)-2′,3′-dihydrospiro[cyclohexane-1,1′-inden]-3′-yl)oxy)-6-methylphenyl)acetate (377b)

Compound 377b was prepared according to the procedure reported in step-3 of scheme-112, from ethyl 2-(2-((5′-bromo-2′,3′-dihydrospiro[cyclohexane-1,1′-inden]-3′-yl)oxy)-6-methylphenyl)acetate (377a) (100 mg, 0.219 mmol) in dioxane/2-MeTHF (2 mL) using 5-(4,4,5,5-tetramethyl-1,3,2-dioxaborolan-2-yl)isoquinolin-1-amine (114a) (118 mg, 0.437 mmol). K₃PO₄ (3 M aqueous solution, 146 μl, 0.437 mmol), PCy₃ (24.52 mg, 0.087 mmol), XPhos (25.8 mg, 0.054 mmol), Pd₂(dba)₃ (40.0 mg, 0.044 mmol) and heating at 100° C. for 10 h to afford after workup and purification using method-U, ethyl 2-(2-((5′-(1-aminoisoquinolin-5-yl)-2′,3′-dihydrospiro[cyclohexane-1,1′-inden]-3′-yl)oxy)-6-methylphenyl)acetate (377b) (57 mg, 50% yield) as a clear oil; MS (ES+): 521.3 (M+1).

### Step-3: Preparation of 2-(2-((5′-(1-aminoisoquinolin-5-yl)-2′,3′-dihydrospiro[cyclohexane-1,1′-inden]-3′-yl)oxy)-6-methylphenyl)acetic acid (377c)

Compound 377c was prepared according to the procedure reported in step-2 of scheme-1, from ethyl 2-(2-((5′-(1-aminoisoquinolin-5-yl)-2′,3′-dihydrospiro[cyclohexane-1,1′-inden]-3′-yl)oxy)-6-methylphenyl)acetate (377b) (57 mg, 0.109 mmol) in THF (3 mL) using lithium hydroxide (15.71 mg, 0.656 mmol) in water (I mL) to afford after work up and purification using method-M, 2-(2-((5′-(1-aminoisoquinolin-5-yl)-2′,3′-dihydrospiro[cyclohexane-1,1′-inden]-3′-yl)oxy)-6-methylphenyl)acetic acid (377c) (35 mg, 32% yield) HCl salt as a white solid; ¹H NMR (300 MHz, DMSO-d₆) δ 13.34 (s, 1H, D₂O exchangeable), 12.04 (s, 1H, D₂O exchangeable), 9.14 (s, 2H, DO exchangeable), 8.60 (d, J=8.2 Hz, 1H), 7.94-7.87 (m, 1H), 7.83 (t, J=7.8 Hz, 1H), 7.62 (d, J=7.3 Hz, 1H), 7.54-7.47 (m, 1H), 7.47-7.40 (m, 1H), 7.40-7.32 (m, 1H), 7.19-7.05 (m, 2H), 6.98 (d, J=7.2 Hz, 1H), 6.80 (d, J=7.1 Hz, 1H), 5.95-5.72 (m, 1H), 3.50 (s, 2H), 2.76-2.61 (m, 1H), 2.20 (s, 3H), 2.00 (dd, J=13.6, 4.7 Hz, 1H), 1.87-1.49 (m, 8H), 1.44-1.19 (m, 2H); MS (ES+): 493.2 (M+1); (ES−): 491.2 (M−1).

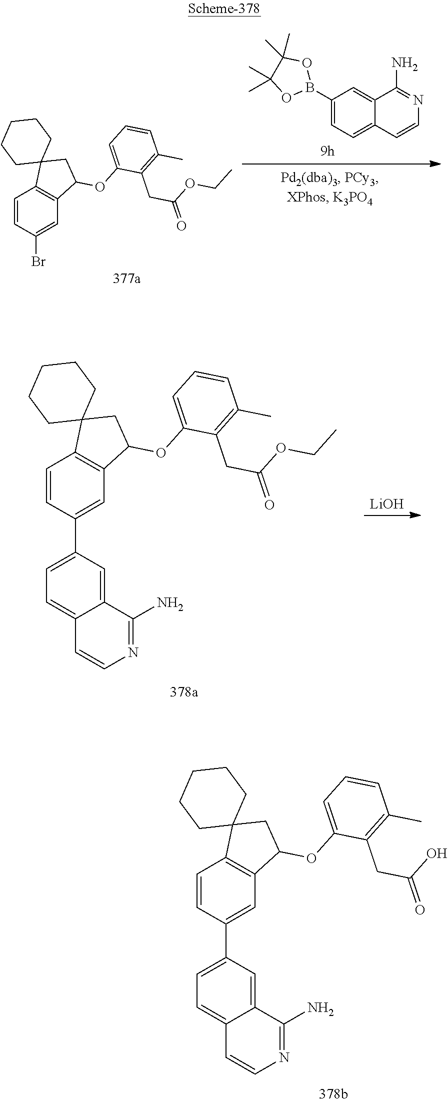 [SMILES: CCOC(=O)Cc1c(C)cccc1OC1CC2(CCCCC2)c2ccc(Br)cc21 ; CC1(C)OB(c2ccc3ccnc(N)c3c2)OC1(C)C ; CCOC(=O)Cc1c(C)cccc1OC1CC2(CCCCC2)c2ccc(-c3ccc4ccnc(N)c4c3)cc21 ; "LiOH" ; Cc1cccc(OC2CC3(CCCCC3)c3ccc(-c4ccc5ccnc(N)c5c4)cc32)c1CC(=O)O]

### Preparation of 2-(2-((5′-(1-aminoisoquinolin-7-yl)-2′,3′-dihydrospiro[cyclohexane-1,1′-inden]-3′-yl)oxy)-6-methylphenyl)acetic acid (378b)

### Step-1: Preparation of ethyl 2-(2-((5′-(1-aminoisoquinolin-7-yl)-2′,3′-dihydrospiro[cyclohexane-1,1′-inden]-3′-yl)oxy)-6-methylphenyl)acetate (378a)

Compound 378a was prepared according to the procedure reported in step-3 of scheme-112, from ethyl 2-(2-((5′-bromo-2′3′-dihydrospiro[cyclohexane-1,1′-inden]-3′-yl)oxy)-6-methylphenyl)acetate (377a) (100 mg, 0.219 mmol) in dioxane/2-MeTHF (2 mL) using 7-(4,4,5,5-tetramethyl-1,3,2-dioxaborolan-2-yl)isoquinolin-1-amine (9h) (118 mg, 0.437 mmol), K₃PO₄ (3M aqueous solution, 146 μl, 0.437 mmol), PCy₃ (24.52 mg, 0.087 mmol), XPhos (20.84 mg, 0.044 mmol), Pd₂(dba)₃ (40.0 mg, 0.044 mmol) and heating at 100° C. for 10 b to afford after workup and purification using method-U, ethyl 2-(2-((5′-(1-aminoisoquinolin-7-yl)-2′,3′-dihydrospiro[cyclohexane-1,1′-inden]-3′-yl)oxy)-6-methylphenyl)acetate (378a) (58 mg, 51% yield) as a clear oil; MS (ES+): 521.3 (M+1).

### Step-2: Preparation of 2-(2-((5′-(1-aminoisoquinolin-7-yl)-2′,3′-dihydrospiro[cyclohexane-1,1′-inden]-3′-yl)oxy)-6-methylphenyl)acetic acid (378b)

Compound 378b was prepared according to the procedure reported in step-2 of scheme-1, from ethyl 2-(2-((5′-(1-aminoisoquinolin-7-yl)-2′,3′-dihydrospiro[cyclohexane-1,1′-inden]-3′-yl)oxy)-6-methylphenyl)acetate (378a) (58 mg, 0.111 mmol) in THF (3 mL) using lithium hydroxide (15.71 mg, 0.656 mmol) in water (1 mL) to afford after work up and purification using method-M, 2-(2-((5′-(1-aminoisoquinolin-7-yl)-2′3′-dihydrospiro[cyclohexane-1,1′-inden]-3′-yl)oxy)-6-methylphenyl)acetic acid (378b) (26 mg, 24% yield) HCl salt as a white solid; ¹H NMR (300 MHz, DMSO-d₆) δ 13.21 (s, 1H, D₂O exchangeable), 12.09 (s, 1H, D₂O exchangeable), 9.08 (s, 2H, D₂O exchangeable), 8.87 (s, 1H), 8.29 (dd, J=8.5, 1.6 Hz, 1H), 8.03 (d, J=8.5 Hz, 1H), 7.89 (dd, J=8.0, 1.8 Hz, 1H), 7.83 (d, J=1.7 Hz, 1H), 7.69 (d, J=6.9 Hz, 1H), 7.52 (d, J=8.0 Hz, 1H), 7.27 (d, J=6.9 Hz, 1H), 7.23-7.10 (m, 2H), 6.82 (d, J=7.3 Hz, 1H), 5.92-5.82 (m, 1H), 3.62-3.48 (m, 2H), 2.69-2.59 (m, 1H), 2.22 (s, 3H), 1.97 (dd, J=13.7, 4.3 Hz, 1H), 1.81-1.45 (m, 8H), 1.41-1.21 (m, 2H); MS (ES+): 493.2 (M+1); (ES−): 491.2 (M−1).

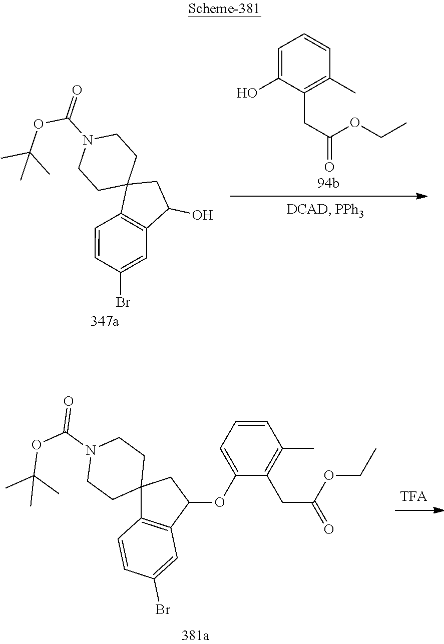 [SMILES: CC(C)(C)OC(=O)N1CCC2(CC1)CC(O)c1cc(Br)ccc12 ; CCOC(=O)Cc1c(C)cccc1O ; CCOC(=O)Cc1c(C)cccc1OC1CC2(CCN(C(=O)OC(C)(C)C)CC2)c2ccc(Br)cc21]

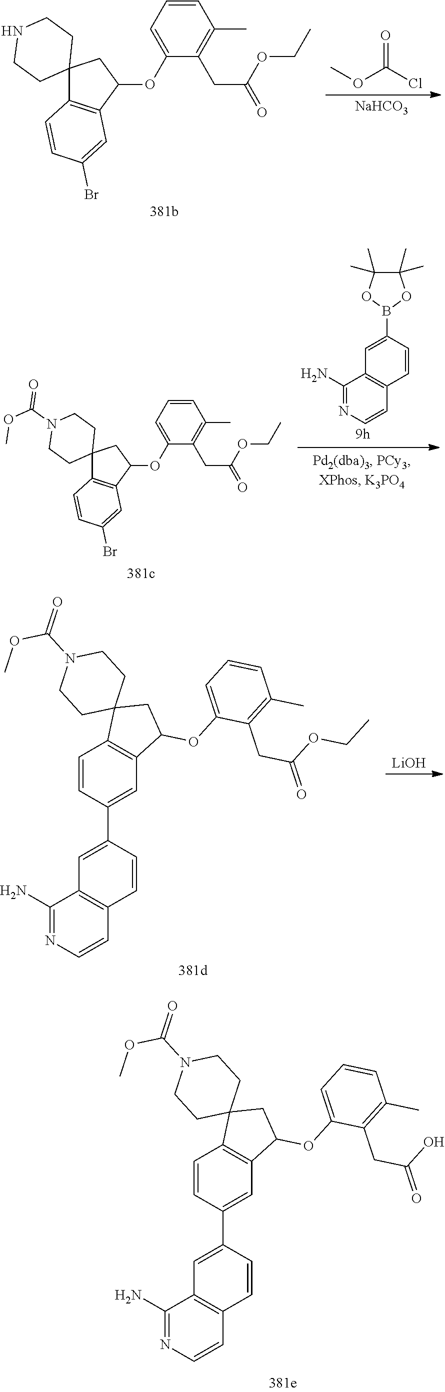

### Preparation of 2-(2-((5-(1-aminoisoquinolin-7-yl)-1′-(methoxycarbonyl)-2,3-dihydrospiro[indene-1,4′-piperidin]-3-yl)oxy)-6-methylphenyl)acetic acid (381e)

### Step-1: Preparation of tert-butyl 5-bromo-3-(2-(2-ethoxy-2-oxoethyl)-3-methylphenoxy)-2,3-dihydrospiro[indene-1,4′-piperidine]-1′-carboxylate (381a)

Compound 381a was prepared according to the procedure reported in step-2 of scheme-2, from tert-butyl 5-bromo-3-hydroxy-2,3-dihydrospiro[indene-1,4′-piperidine]-1′-carboxylate (347a) (4.6 g, 12.03 mmol) in DCM (210 mL) using PPh₃ (3.79 g, 14.44 mmol), ethyl 2-(2-hydroxy-6-methylphenyl)acetate (94b) (2.80 g, 14.44 mmol) and a solution of DCAD (5.30 g, 14.44 mmol) in DCM (70 mL) to afford after workup and purification using method-BT, tert-butyl 5-bromo-3-(2-(2-ethoxy-2-oxoethyl)-3-methylphenoxy)-2,3-dihydrospiro[indene-1,4′-piperidine]-1′-carboxylate (381a) (2.9 g, 43% yield) as a white solid; MS (ES+): 580.2 (M+Na).

### Step-2: Preparation of ethyl 2-(2-((5-bromo-2,3-dihydrospiro[indene-1,4′-piperidin]-3-yl)oxy)-6-methylphenyl)acetate (381b)

Compound 381b was prepared according to the procedure reported in step-4 of scheme-9, from tert-butyl 5-bromo-3-(2-(2-ethoxy-2-oxoethyl)-3-methylphenoxy)-2,3-dihydrospiro[indene-1,4′-piperidine]-1′-carboxylate (381a) (2.8 g, 5.01 mmol) in DCM (30 mL) using TFA (2.70 mL, 35.1 mmol) and stirring at RT for 2 h to afford after workup and purification using method-BU, ethyl 2-(2-((5-bromo-2,3-dihydrospiro[indene-1,4′-piperidin]-3-yl)oxy)-6-methylphenyl)acetate, TFA (381b) (2.25 g, 78% yield) and was used as such without further purification; MS (ES+): 458.2 (M+1).

### Step-3: Preparation of methyl 5-bromo-3-(2-(2-ethoxy-2-oxoethyl)-3-methylphenoxy)-2,3-dihydrospiro[indene-1,4′-piperidine]-1′-carboxylate (381c)

Compound 381c was prepared according to the procedure reported in step-1 of scheme-126, from ethyl 2-(2-((5-bromo-2,3-dihydrospiro[indene-1,4′-piperidin]-3-yl)oxy)-6-methylphenyl)acetate. TFA (381b) (0.572 g, 0.999 mmol) in THF (10 mL) using sodium bicarbonate (2.498 mL, 5.00 mmol) and methyl carbonochloridate (0.116 mL, 1.499 mmol) to afford after workup and purification using method-P, methyl 5-bromo-3-(2-(2-ethoxy-2-oxoethyl)-3-methylphenoxy)-2,3-dihydrospiro[indene-1,4′-piperidine]-1′-carboxylate (381c) (0.45 g, 87% yield) as a clear oil; MS (ES+): 538.1 (M+Na).

### Step-4: Preparation of methyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (381d)

Compound 381d was prepared according to the procedure reported in step-3 of scheme-112, from methyl 5-bromo-3-(2-(2-ethoxy-2-oxoethyl)-3-methylphenoxy)-2,3-dihydrospiro[indene-1,4′-piperidine]-1′-carboxylate (381c) (220 mg, 0.426 mmol) in dioxane/2-MeTHF (2 mL) using 7-(4,4,5,5-tetramethyl-1,3,2-dioxaborolan-2-yl)isoquinolin-1-amine (9h) (230 mg, 0.852 mmol), K₃PO₄ (3M aqueous solution, 284 μl, 0.852 mmol), PCy₃ (47.8 mg, 0.170 mmol), XPhos (20.31 mg, 0.043 mmol), Pd₂(dba)₃ (78 mg, 0.085 mmol) and heating at 100° C. for 10 h to afford after workup and purification using method-BV, methyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (381d) (41 mg, 17% yield) as a white solid; MS (ES+): 580.3 (M+1).

### Step-5: Preparation of 2-(2-((5-(1-aminoisoquinolin-7-yl)-1′-(methoxycarbonyl)-2,3-dihydrospiro[indene-1,4′-piperidin]-3-yl)oxy)-6-methylphenyl)acetic acid (381e)

Compound 381e was prepared according to the procedure reported in step-2 of scheme-1, from methyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (381d) (40 mg, 0.069 mmol) in MeOH (1 ml) and THF (1 mL) using lithium hydroxide (1 N solution, 207 μl, 0.207 mmol) to afford after work up and purification using method-BV, 2-(2-((5-(1-aminoisoquinolin-7-yl)-1′-(methoxycarbonyl)-2,3-dihydrospiro[indene-1,4′-piperidin]-3-yl)oxy)-6-methylphenyl)acetic acid (381e) (20 mg, 53% yield) HCl salt as a white solid; ¹H NMR (300 MHz, DMSO-d₆) & 13.16 (s, 1H, D₂O exchangeable), 12.11 (s, 1H, D₂O exchangeable), 9.08 (s, 2H, D₂O exchangeable), 8.88 (s, 1H), 8.31 (dd. J=8.5, 1.6 Hz, 1H), 8.04 (d, J=8.5 Hz, 1H), 7.95-7.81 (m, 2H), 7.69 (d, J=6.9 Hz, 1H), 7.57 (d, J=8.0 Hz, 1H), 7.32-7.08 (m, 3H), 6.86-6.77 (m, 1H), 5.99-5.84 (m, 1H), 4.17-3.90 (m, 2H), 3.62 (s, 3H), 3.55-3.42 (m, 2H), 3.18-2.77 (m, 2H), 2.69 (dd, J=13.7, 6.7 Hz, 1H), 2.22 (s, 3H), 2.12-2.01 (m, 1H), 1.97-1.69 (m, 2H), 1.67-1.42 (m, 2H); MS (ES+): 552.3 (M+1); (ES−): 550.2 (M−1).

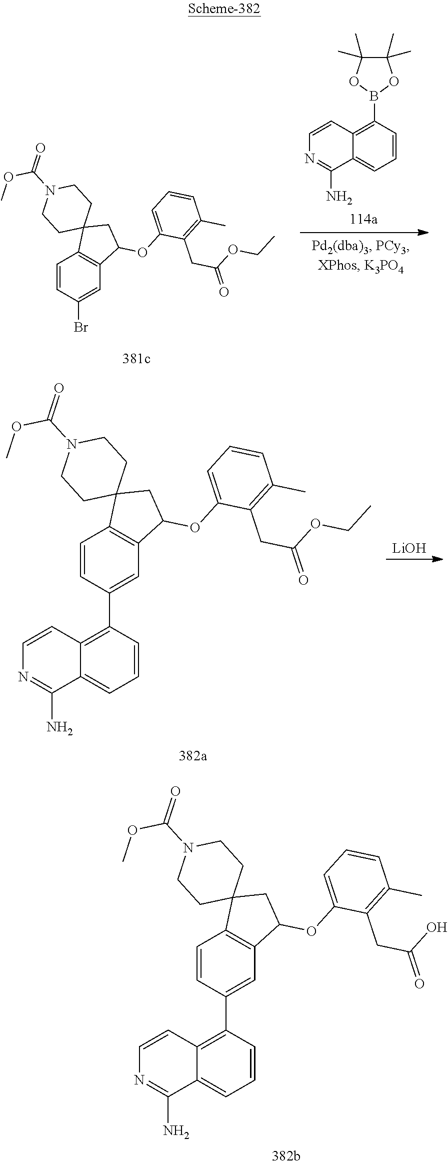

### Preparation of 2-(2-((5-(1-aminoisoquinolin-5-yl)-1′-(methoxycarbonyl)-2,3-dihydrospiro[indene-1,4′-piperidin]-3-yl)oxy)-6-methylphenyl)acetic acid (382b)

### Step-1: Preparation of methyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (382a)

Compound 382a was prepared according to the procedure reported in step-3 of scheme-112, from methyl 5-bromo-3-(2-(2-ethoxy-2-oxoethyl)-3-methylphenoxy)-2,3-dihydrospiro[indene-1,4′-piperidine]-1′-carboxylate (381c) (220 mg, 0.426 mmol) in dioxane/2-MeTHF (2 mL) using 5-(4,4,5,5-tetramethyl-1,3,2-dioxaborolan-2-yl)isoquinolin-1-amine (114a) (230 mg, 0.852 mmol), K₃PO₄ (3M aqueous solution, 284 μl, 0.852 mmol), PCy₃ (47.8 mg, 0.170 mmol), XPhos (20.31 mg, 0.043 mmol), Pd₂(dba)₃ (78 mg, 0.085 mmol) and heating at 100° C. for 10 h to afford after workup and purification using method-BV, methyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (382a) (90 mg, 36% yield) as a white solid; MS (ES+): 580.3 (M+1).

Step-2: Preparation of 2-(2-((5-(1-aminoisoquinolin-5-yl)-1′-(methoxycarbonyl)-2,3-dihydrospiro[indene-1,4′-piperidin]-3-yl)oxy)-6-methylphenyl)acetic acid (382b) Compound 382b was prepared according to the procedure reported in step-2 of scheme-1, from methyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (382a) (65 mg, 0.112 mmol) in MeOH (1 mL) and THF (1 mL) using lithium hydroxide (1 N solution, 336 μl, 0.336 mmol) to afford after work up and purification using method-BV, 2-(2-((5-(1-aminoisoquinolin-5-yl)-1′-(methoxycarbonyl)-2,3-dihydrospiro[indene-1,4′-piperidin]-3-yl)oxy)-6-methylphenyl)acetic acid (382b) (53 mg, 86% yield) HCl salt as a pale yellow solid. ¹H NMR (300 MHz, DMSO-d₆) δ 13.41 (s, 1H, D₂O exchangeable), 12.05 (s, 1H, D₂O exchangeable), 9.20 (s, 2H, D₂O exchangeable), 8.62 (d, J=8.2 Hz, 1H), 7.94-7.79 (m, 2H), 7.62 (d, J=7.2 Hz, 1H), 7.55 (d, J=7.9 Hz, 1H), 7.47-7.35 (m, 2H), 7.20-7.07 (m, 2H), 6.96 (d, J=7.3 Hz, 1H), 6.80 (dd, J=6.6, 2.1 Hz, 1H), 5.99-5.79 (m, 1H), 4.03 (s, 2H), 3.63 (s, 3H), 3.50 (s, 2H), 3.17-2.85 (m, 2H), 2.76 (dd, J=13.7, 6.9 Hz, 1H), 2.20 (s, 3H), 2.15-2.01 (m, 1H), 2.01-1.73 (m, 2H), 1.73-1.49 (m, 2H); MS (ES+): 552.3 (M+1); (ES−): 550.2 (M−1).

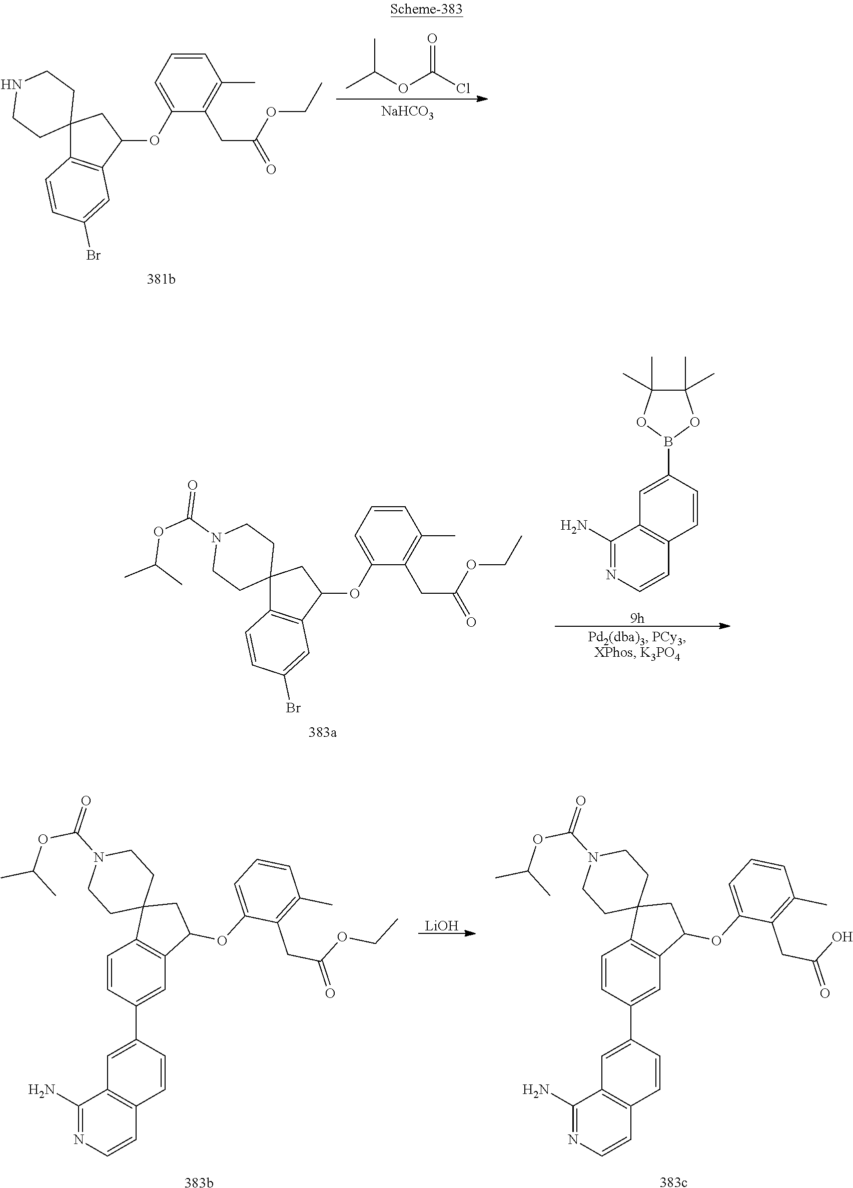

### Preparation of 2-(2-((5-(1-aminoisoquinolin-7-yl)-1′-(isopropoxycarbonyl)-2,3-dihydrospiro[indene-1,4′-piperidin]-3-yl)oxy)-6-methylphenyl)acetic acid (383c)

### Step-1: Preparation of isopropyl 5-bromo-3-(2-(2-ethoxy-2-oxoethyl)-3-methylphenoxy)-2,3-dihydrospiro[indene-1,4′-piperidine]-1′-carboxylate (383a)

Compound 383a was prepared according to the procedure reported in step-1 of scheme-126, from ethyl 2-(2-((5-bromo-2,3-dihydrospiro[indene-1,4′-piperidin]-3-yl)oxy)-6-methylphenyl)acetate, TFA (381b) (572 mg, 0.999 mmol) in THF (10 mL) using sodium bicarbonate (2498 μl, 5.00 mmol) and isopropyl carbonochloridate (1499 μl, 1.499 mmol) to afford after workup and purification using method-P, isopropyl 5-bromo-3-(2-(2-ethoxy-2-oxoethyl)-3-methylphenoxy)-2,3-dihydrospiro[indene-1,4′-piperidine]-1′-carboxylate (383a) (0.47 g, 86% yield) as a clear oil; MS (ES+): 566.1 (M+Na).

### Step-2: Preparation of isopropyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (383b)

Compound 383b was prepared according to the procedure reported in step-3 of scheme-112, from isopropyl 5-bromo-3-(2-(2-ethoxy-2-oxoethyl)-3-methylphenoxy)-2,3-dihydrospiro[indene-1,4′-piperidine]-1′-carboxylate (383a) (230 mg, 0.422 mmol) in dioxane/2-MeTHF (2 mL) using 7-(4,4,5,5-tetramethyl-1,3,2-dioxaborolan-2-yl)isoquinolin-1-amine (9h) (228 mg, 0.845 mmol), K₃PO₄ (3M aqueous solution, 282 μl, 0.845 mmol), PCy₃ (47.4 mg, 0.169 mmol), XPhos (20.14 mg, 0.042 mmol), Pd₂(dba)₃ (77 mg, 0.084 mmol) and heating at 100° C. for 10 h to afford after workup and purification using method-BV, isopropyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (383b) (52 mg, 20.26% yield) as a white solid; MS (ES+): 608.3 (M+1).

### Step-3: Preparation of 2-(2-((5-(1-aminoisoquinolin-7-yl)-1′-(isopropoxycarbonyl)-2,3-dihydrospiro[indene-1,4′-piperidin]-3-yl)oxy)-6-methylphenyl)acetic acid (383c)

Compound 383c was prepared according to the procedure reported in step-2 of scheme-1, from isopropyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (383b) (50 mg, 0.082 mmol) in MeOH (1 mL) and THF (1 mL) using lithium hydroxide (1 N solution, 247 μl, 0.247 mmol) to afford after work up and purification using method-BV, 2-(2-((5-(1-aminoisoquinolin-7-yl)-1′-(isopropoxycarbonyl)-2,3-dihydrospiro[indene-1,4′-piperidin]-3-yl)oxy)-6-methylphenyl)acetic acid (383c) (25 mg, 52% yield) HCl salt as an off white solid. ¹H NMR (300 MHz, DMSO-d₆) δ 13.22 (s, 1H, D₂O exchangeable), 12.12 (s, 1H, D₂O exchangeable), 9.12 (s, 2H, D₂O exchangeable), 8.95-8.84 (m, 1H), 8.31 (dd, J=8.5, 1.6 Hz, 1H), 8.04 (d, J=8.6 Hz, 1H), 7.96-7.81 (m, 2H), 7.69 (d, J=6.9 Hz, 1H), 7.57 (d, J=8.0 Hz, 1H), 7.34-7.04 (m, 3H), 6.83 (dd, J=7.2, 1.7 Hz, 1H), 5.96-5.85 (m, 1H), 4.85-4.74 (m, 1H), 4.16-3.83 (m, 2H), 3.62-3.40 (m, 2H), 3.15-2.77 (m, 2H), 2.75-2.60 (m, 1H), 2.22 (s, 3H), 2.13-1.98 (m, 1H), 1.97-1.34 (m, 4H), 1.21 (d, J=6.2 Hz, 6H); MS (ES+): 580.3 (M+1); (ES−): 578.2 (M−1).

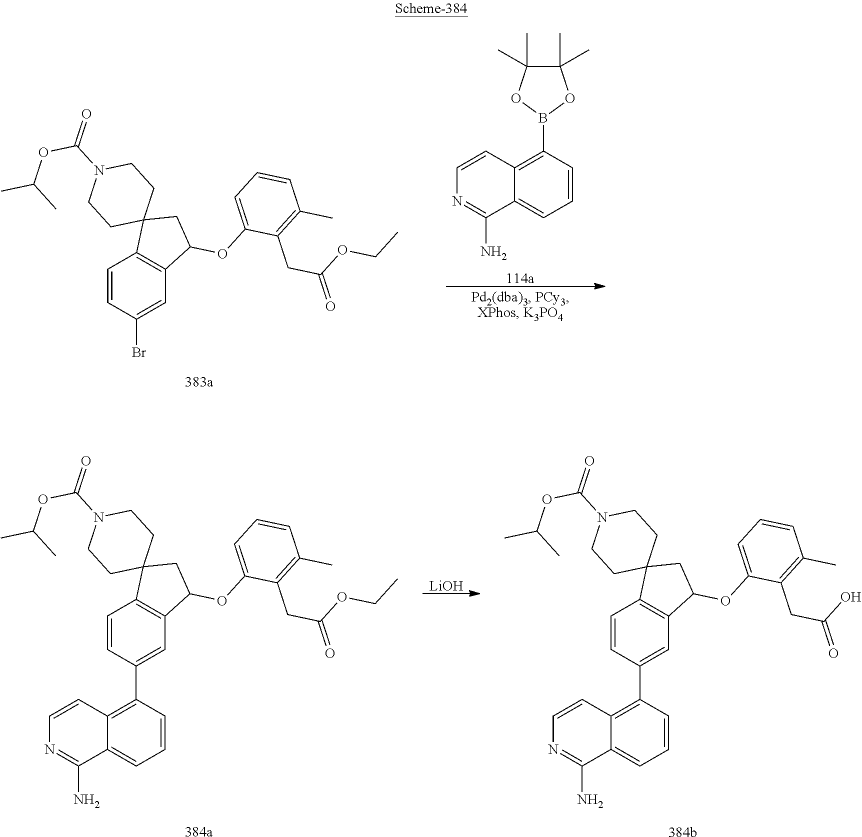

### Preparation of 2-(2-((5-(1-aminoisoquinolin-5-yl)-1′-(isopropoxycarbonyl)-2,3-dihydrospiro[indene-1,4′-piperidin]-3-yl)oxy)-6-methylphenyl)acetic acid (384b)

### Step-1: Preparation of isopropyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (384a)

Compound 384a was prepared according to the procedure reported in step-3 of scheme-112, from isopropyl 5-bromo-3-(2-(2-ethoxy-2-oxoethyl)-3-methylphenoxy)-2,3-dihydrospiro[indene-1,4′-piperidine]-1′-carboxylate (383a) (230 mg, 0.422 mmol) in dioxane/2-MeTHF (2 mL) using 5-(4,4,5,5-tetramethyl-1,3,2-dioxaborolan-2-yl)isoquinolin-1-amine (114a) (228 mg, 0.845 mmol), K₃PO₄ (3 M aqueous solution, 282 μl, 0.845 mmol), PCy₃ (47.4 mg, 0.169 mmol), XPhos (20.14 mg, 0.042 mmol), Pd₂(dba)₃ (77 mg, 0.084 mmol) and beating at 100° C. for 10 h to afford after workup and purification using method-BV, 5-(1-aminoisoquinolin-5-yl)-3-(2-(2-ethoxy-2-oxoethyl)-3-methylphenoxy)-2,3-isopropyl dihydrospiro[indene-1,4′-piperidine]-1′-carboxylate (384a) (121 mg, 47% yield) as a pale yellow solid; MS (ES+): 608.3 (M+1).

### Step-2: Preparation of 2-(2-((5-(1-aminoisoquinolin-5-yl)-1′-(isopropoxycarbonyl)-2,3-dihydrospiro[indene-1,4′-piperidin]-3-yl)oxy)-6-methylphenyl)acetic acid (384b)

Compound 384b was prepared according to the procedure reported in step-2 of scheme-1, from isopropyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (384a) (95 mg, 0.156 mmol) in MeOH (1 mL) and THF (1 mL) using lithium hydroxide (1 N solution, 469 μl, 0.469 mmol) to afford after work up and purification using method-BV, 2-(2-((5-(1-aminoisoquinolin-5-yl)-1′-(isopropoxycarbonyl)-2,3-dihydrospiro[indene-1,4′-piperidin]-3-yl)oxy)-6-methylphenyl)acetic acid (384b) (69 mg, 76% yield) HCl salt as a white solid; ¹H NMR (300 MHz, DMSO-d₆) δ 13.24 (s, 1H, D₂O exchangeable), 12.05 (s, 1H, D₂O exchangeable), 9.13 (s, 2H, D₂O exchangeable), 8.60 (d, J=8.2 Hz, 1H), 7.95-7.88 (m, 1H), 7.88-7.78 (m, 1H), 7.67-7.51 (m, 2H), 7.48-7.37 (m, 2H), 7.21-7.08 (m, 2H), 6.96 (d, J=7.3 Hz, 1H), 6.85-6.76 (m, 1H), 5.91 (t, J=5.5 Hz, 1H), 4.89-4.73 (m, 1H), 4.16-3.84 (m, 2H), 3.50 (s, 2H), 3.19-2.83 (m, 2H), 2.76 (dd, J=13.8, 7.0 Hz, 1H), 2.20 (s, 3H), 2.10 (dd, J=13.7, 4.4 Hz, 1H), 2.01-1.49 (m, 4H), 1.21 (d, J=6.2 Hz, 6H); MS (ES+): 580.3 (M+1); (ES−): 578.2 (M−1).

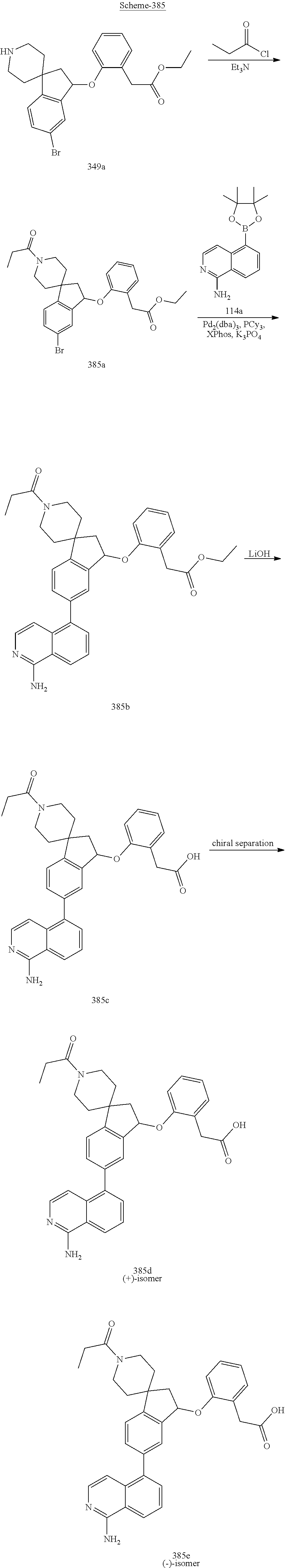

### Preparation of 2-(2-((5-(1-aminoisoquinolin-5-yl)-1′-propionyl-2,3-dihydrospiro[indene-1,4′-piperidin]-3-yl)oxy)phenyl)acetic acid (385c), (+)-2-(2-((5-(1-aminoisoquinolin-5-yl)-1′-propionyl-2,3-dihydrospiro[indene-1,4′-piperidin]-3-yl)oxy)phenyl)acetic acid (385d) and (−)-2-(2-((5-(1-aminoisoquinolin-5-yl)-1′-propionyl-2,3-dihydrospiro[indene-1,4′-piperidin]-3-yl)oxy)phenyl)acetic acid (385e)

### Step-1: Preparation of ethyl 2-(2-((5-bromo-1′-propionyl-2,3-dihydrospiro[indene-1,4′-piperidin]-3-yl)oxy)phenyl)acetate (385a)

To an ice-cold solution of ethyl 2-(2-((5-bromo-2,3-dihydrospiro[indene-1,4′-piperidin]-3-yl)oxy)phenyl)acetate (349a) (0.6 g, 1.35 mmol) in DCM (10 mL) was added triethylamine (0.414 mL, 2.97 mmol), propionyl chloride (0.153 mL, 1.755 mmol) and the reaction mixture was allowed to warm to RT over a period of 5 h. The solvent was removed, and the obtained residue was purified using method-P, to give ethyl 2-(2-((5-bromo-1′-propionyl-2,3-dihydrospiro[indene-1,4′-piperidin]-3-yl)oxy)phenyl)acetate (385a) (0.4 g, 59% yield) as clear oil; MS (ES+): 500.1 (M+1).

### Step-2: Preparation of ethyl 2-(2-((5-(1-aminoisoquinolin-5-yl)-1′-propionyl-2,3-dihydrospiro[indene-1,4′-piperidin]-3-yl)oxy)phenyl)acetate (385b)

Compound 385b was prepared according to the procedure reported in step-3 of scheme-112, from ethyl 2-(2-((5-bromo-1′-propionyl-2,3-dihydrospiro[indene-1,4′-piperidin]-3-yloxy)phenyl)acetate (385a) (200 mg, 0.400 mmol) in dioxane/2-MeTHF (2 mL) using 5-(4,4,5,5-tetramethyl-1,3,2-dioxaborolan-2-yl)isoquinolin-1-amine (114a) (216 mg, 0.799 mmol), K₃PO₄ (3M aqueous solution, 266 μl, 0.799 mmol), PCy₃ (44.8 mg, 0.16 mmol), XPhos (19.05 mg, 0.04 mmol). Pd₂(dba)₃ (73.2 mg, 0.08 mmol) and heating at 100° C. for 10 h to afford after workup and purification using method-BV, ethyl 2-(2-((5-(1-aminoisoquinolin-5-yl)-1′-propionyl-2,3-dihydrospiro[indene-1,4′-piperidin]-3-yl)oxy)phenyl)acetate (385b) (161 mg, 72% yield) as a white solid; MS (ES+): 564.4 (M+1).

### Step-3: Preparation of 2-(2-((5-(1-aminoisoquinolin-5-yl)-1′-propionyl-2,3-dihydrospiro[indene-1,4′-piperidin]-3-yl)oxy)phenyl)acetic acid (385c)

Compound 385c was prepared according to the procedure reported in step-2 of scheme-1, from ethyl 2-(2-((5-(1-aminoisoquinolin-5-yl)-1′-propionyl-2,3-dihydrospiro[indene-1,4′-piperidin]-3-yl)oxy)phenyl)acetate (385b) (140 mg, 0.248 mmol) in MeOH (1.5 mL) and THF (1.5 mL) using lithium hydroxide (1 N solution, 745 μl, 0.745 mmol) to afford after work up and purification using method-BV, 2-(2-((5-(1-aminoisoquinolin-5-yl)-1′-propionyl-2,3-dihydrospiro[indene-1,4′-piperidin]-3-yl)oxy)phenyl)acetic acid (385c) (65 mg, 49% yield) HCl salt as a white solid; ¹H NMR (300 MHz, DMSO) δ 13.38 (s, 1H, D₂O exchangeable), 12.02 (s, 1H, D₂O exchangeable), 9.21 (s, 2H, D₂O exchangeable), 8.62 (d, J=8.2 Hz, 1H), 7.96-7.78 (m, 2H), 7.62 (d, J=7.3 Hz, 1H), 7.57-7.50 (m, 1H), 7.48-7.36 (m, 2H), 7.35-7.12 (m, 3H), 7.02-6.85 (m, 2H), 6.00-5.76 (m, 1H), 4.54-4.40 (m, 1H), 3.90 (t, J=13.2 Hz, 1H), 3.45 (s, 2H), 3.16 (t, J=13.1 Hz, 1H), 2.95-2.58 (m, 2H), 2.45-2.27 (m, 2H), 2.12 (dd, J=13.6, 4.5 Hz, 1H), 2.04-1.46 (m, 4H), 1.03 (t, J=7.4 Hz, 3H); MS (ES+): 536.3 (M+1); (ES−): 534.2 (M−1).

### Step-4: Preparation of (+)-2-(2-((5-(1-aminoisoquinolin-5-yl)-1′-propionyl-2,3-dihydrospiro[indene-1,4′-piperidin]-3-yl)oxy)phenyl)acetic acid (385d) and (−)-2-(2-((5-(1-aminoisoquinolin-5-yl)-1′-propionyl-2,3-dihydrospiro[indene-1,4′-piperidin]-3-yl)oxy)phenyl)acetic acid (385e)

Compounds 385d and 385e was obtained by purification of 2-(2-((5-(1-aminoisoquinolin-5-yl)-1′-propionyl-2,3-dihydrospiro[indene-1,4′-piperidin]-3-yl)oxy)phenyl)acetic acid (385c) using chiral HPLC. [Diacel CHIRAL PAK-IA (250*30 mm, 5μ), Mobile phase A: 0.1% TFA in n-hexane, Mobile phase B: DCM:MeOH (1:1), Isocratic (A:B): 70:30, Flowrate: 30 mL/min] to give (+)-2-(2-((5-(1-aminoisoquinolin-5-yl)-1′-propionyl-2,3-dihydrospiro[indene-1,4′-piperidin]-3-yl)oxy)phenyl)acetic acid, TFA salt (385d) as a white solid; ¹H NMR (300 MHz, DMSO-d₆) δ 13.11 (s, 1H, D₂O exchangeable), 9.12 (s, 2H, D₂O exchangeable), 8.58 (d, J=8.3 Hz, 1H), 7.97-7.80 (m, 2H), 7.63-7.49 (m, 2H), 7.49-7.37 (m, 2H), 7.35-7.16 (m, 3H), 7.02-6.84 (m, 2H), 6.00-5.91 (m, 1H), 4.57-4.41 (m, 1H), 3.90 (s, 1H), 3.45 (s, 2H), 3.36-3.06 (m, 1H), 2.94-2.59 (m, 2H), 2.38 (s, 2H), 2.20-1.55 (m, 5H), 1.03 (t, J=7.3 Hz, 3H); ¹⁹F NMR (282 MHz, DMSO-d₆) δ −74.12; [α]D=+23.808 (C=0.25, MeOH); MS (ES+): 536.20 (M+1) and (−)-2-(2-((5-(1-aminoisoquinolin-5-yl)-1′-propionyl-2,3-dihydrospiro[indene-1,4′-piperidin]-3-yl)oxy)phenyl)acetic acid, TFA salt (385e) as a white solid; H NMR (300 MHz, DMSO-d₆) δ 13.08 (s, 1H, D₂O exchangeable), 9.11 (s, 2H, D₂O exchangeable), 8.57 (d, J=8.2 Hz, 1H), 7.96-7.78 (m, 2H), 7.63-7.49 (m, 2H), 7.46-7.38 (m, 2H), 7.34-7.22 (m, 2H), 7.22-7.16 (m, 1H), 7.04-6.86 (m, 2H), 6.00-5.90 (m, 1H), 4.55-4.39 (m, 1H), 3.98-3.81 (m, 1H), 3.45 (s, 2H), 3.36-3.07 (m, 1H), 2.92-2.60 (m, 2H), 2.38 (t, J=6.1 Hz, 2H), 2.19-1.51 (m, 5H), 1.03 (t, J=7.4 Hz, 3H); [α]D=˜ 22.448 (C=0.25, MeOH); MS (ES+): 536.20 (M+1).

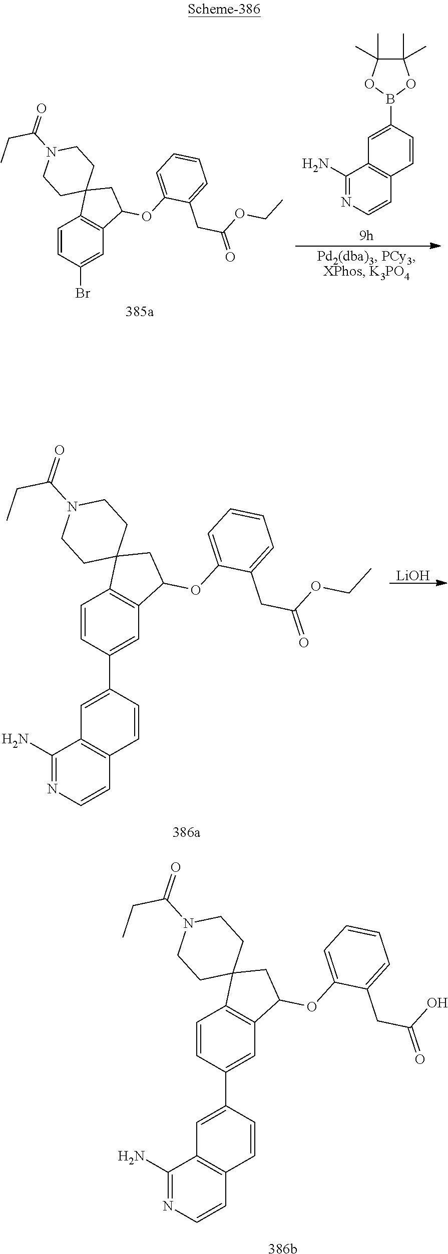

### Preparation of 2-(2-((5-(1-aminoisoquinolin-7-yl)-1′-propionyl-2,3-dihydrospiro[indene-1,4′-piperidin]-3-yl)oxy)phenyl)acetic acid (386b)

### Step-1: Preparation of ethyl 2-(2-((5-(1-aminoisoquinolin-7-yl)-1′-propionyl-2,3-dihydrospiro[indene-1,4′-piperidin]-3-yl)oxy)phenyl)acetate (386a)

Compound 386a was prepared according to the procedure reported in step-3 of scheme-112, from ethyl 2-(2-((5-bromo-1′-propionyl-2,3-dihydrospiro[indene-1,4′-piperidin]-3-yl)oxy)phenyl)acetate (385a) (200 mg, 0.4 mmol) in dioxane/2-MeTHF (2 mL) using 7-(4,4,5,5-tetramethyl-1,3,2-dioxaborolan-2-yl)isoquinolin-1-amine (9h) (216 mg, 0.8 mmol), K₃PO₄ (3 M aqueous solution, 266 μl, 0.8 mmol), PCy₃ (44.8 mg, 0.16 mmol), XPhos (19.05 mg, 0.04 mmol) and Pd₂(dba)₃ (73.2 mg, 0.08 mmol) and heating at 100° C. for 10 h to afford after workup and purification using method-F, ethyl 2-(2-((5-(1-aminoisoquinolin-7-yl)-1′-propionyl-2,3-dihydrospiro[indene-1,4′-piperidin]-3-yl)oxy)phenyl)acetate (386a)(43 mg, 19% yield) as a clear gel; MS (ES+): 564.3 (M+1).

### Step-2: Preparation of 2-(2-((5-(1-aminoisoquinolin-7-yl)-1′-propionyl-2,3-dihydrospiro[indene-1,4′-piperidin]-3-yl)oxy)phenyl)acetic acid (386b)

Compound 386b was prepared according to the procedure reported in step-2 of scheme-1, from ethyl 2-(2-((5-(1-aminoisoquinolin-7-yl)-1′-propionyl-2,3-dihydrospiro[indene-1,4′-piperidin]-3-yl)oxy)phenyl)acetate (386a) (40 mg, 0.071 mmol) in MeOH (0.5 mL) and THF (0.5 mL) using lithium hydroxide (1N solution, 213 μl, 0.213 mmol) to afford after work up and purification using method-BV, 2-(2-((5-(1-aminoisoquinolin-7-yl)-1′-propionyl-2,3-dihydrospiro[indene-1,4′-piperidin]-3-yl)oxy)phenyl)acetic acid (386b) (19 mg, 50% yield) HCl salt as an off white solid; ¹H NMR (300 MHz, DMSO-d₆) δ 13.17 (s, 1H, D₂O exchangeable), 12.06 (s, 1H, D₂O exchangeable), 9.11 (s, 2H, D₂O exchangeable), 8.89 (d, J=1.7 Hz, 1H), 8.32 (dd, J=8.5, 1.6 Hz, 1H), 8.04 (d, J=8.5 Hz, 1H), 7.95-7.84 (m, 2H), 7.69 (d, J=6.9 Hz, 1H), 7.55 (d, J=8.0 Hz, 1H), 7.38-7.15 (m, 4H), 7.00-6.87 (m, 1H), 6.01-5.91 (m, 1H), 4.58-4.36 (m, 1H), 3.98-3.77 (m, 1H), 3.47 (d, J=3.3 Hz, 2H), 3.20-3.03 (m, 1H), 2.89-2.57 (m, 2H), 2.44-2.29 (m, 2H), 2.20-2.04 (m, 1H), 2.00-1.46 (m, 4H), 1.03 (t, J==7.3 Hz, 3H); MS (ES+): 536.2 (M+1); (ES−): 534.2 (M−1).

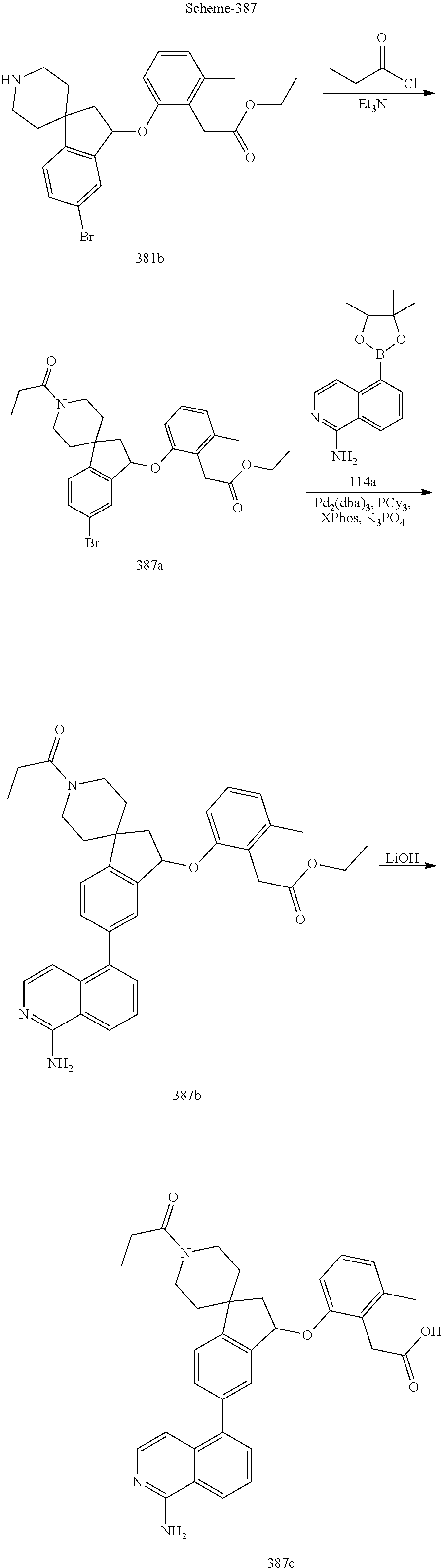

### Preparation of 2-(2-((5-(1-aminoisoquinolin-5-yl)-1′-propionyl-2,3-dihydrospiro[indene-1,4′-piperidin]-3-yl)oxy)-6-methylphenyl)acetic acid (387c)

### Step-1: Preparation of ethyl 2-(2-((5-bromo-1′-propionyl-2,3-dihydrospiro[indene-1,4′-piperidin]-3-yl)oxy)-6-methylphenyl)acetate (387a)

Compound 387a was prepared according to the procedure reported in step-1 of scheme-385, from ethyl 2-(2-((5-bromo-2,3-dihydrospiro[indene-1,4′-piperidin]-3-yl)oxy)-6-methylphenyl)acetate (381b) (0.57 g, 1.243 mmol) in DCM (10 mL) using triethylamine (0.381 mL, 2.74 mmol) and propionyl chloride (0.141 mL, 1.617 mmol) to afford after work up and purification using method-P, to give ethyl 2-(2-((5-bromo-1′-propionyl-2,3-dihydrospiro[indene-1,4′-piperidin]-3-yl)oxy)-6-methylphenyl)acetate (387a) (0.45 g, 70% yield) as a clear oil; MS (ES+): 514.2 (M+1).

### Step-2: Preparation of ethyl 2-(2-((5-(1-aminoisoquinolin-5-yl)-1′-propionyl-2,3-dihydrospiro[indene-1,4′-piperidin]-3-yl)oxy)-6-methylphenyl)acetate (387b)

Compound 387b was prepared according to the procedure reported in step-3 of scheme-112, from ethyl 2-(2-((5-bromo-1′-propionyl-2,3-dihydrospiro[indene-1,4′-piperidin]-3-yl)oxy)-6-methylphenyl)acetate (387a) (220 mg, 0.428 mmol) in dioxane/2-MeTHF (2 mL) using 5-(4,4,5,5-tetramethyl-1,3,2-dioxaborolan-2-yl)isoquinolin-1-amine (114a) (231 mg, 0.855 mmol). K₃PO₄ (3 M aqueous solution, 285 μl, 0.855 mmol), PCy₃ (48 mg, 0.171 mmol), XPhos (20 mg, 0.043 mmol) and Pd₂(dba)₃ (78 mg, 0.086 mmol) and heating at 100° C. for 10 h to afford after workup and purification using method-BV, ethyl 2-(2-((5-(1-aminoisoquinolin-5-yl)-1′-propionyl-2,3-dihydrospiro[indene-1,4′-piperidin]-3-yl)oxy)-6-methylphenyl)acetate (387b) (55 mg, 22% yield) as a white solid; MS (ES+), 578.3 (M+1).

### Step-3: Preparation of 2-(2-((5-(1-aminoisoquinolin-5-yl)-1′-propionyl-2,3-dihydrospiro[indene-1,4′-piperidin]-3-yl)oxy)-6-methylphenyl)acetic acid (387c)

Compound 387c was prepared according to the procedure reported in step-2 of scheme-1, from ethyl 2-(2-((5-(1-aminoisoquinolin-5-yl)-1′-propionyl-2,3-dihydrospiro[indene-1,4′-piperidin]-3-yl)oxy)-6-methylphenyl)acetate (387b) (45 mg, 0.078 mmol) in MeOH (1 mL) and THF (1 mL) using lithium hydroxide (1 N solution, 389 μl, 0.389 mmol) to afford after work up and purification using method-BV, 2-(2-((5-(1-aminoisoquinolin-5-yl)-1′-propionyl-2,3-dihydrospiro[indene-1,4′-piperidin]-3-yl)oxy)-6-methylphenyl)acetic acid (387c) (23 mg, 54% yield) HCl salt as a white solid; ¹H NMR (300 MHz, DMSO-d₆) δ 13.20 (s, 1H. DO exchangeable), 12.01 (s, 1H, D₂O exchangeable), 9.07 (s, 2H, D₂O exchangeable), 8.59 (d, J=8.2 Hz, 1H), 7.94-7.78 (m, 2H), 7.61 (d, J=7.2 Hz, 1H), 7.57-7.48 (m, 1H), 7.46-7.37 (m, 2H), 7.20-7.08 (m, 2H), 6.97 (d, J=7.3 Hz, 1H), 6.81 (dd, J=6.3, 2.2 Hz, 1H), 5.97-5.87 (m, 1H), 4.54-4.37 (m, 1H), 3.89 (t, J=13.9 Hz, 1H), 3.50 (s, 2H), 3.29-3.06 (m, 1H), 2.90-2.59 (m, 2H), 2.44-2.29 (m, 2H), 2.21 (s, 3H), 2.18-2.04 (m, 1H), 2.02-1.42 (m, 4H), 1.03 (t, J=7.4 Hz, 3H); MS (ES+): 550.3 (M+1); (ES−): 548.3 (M−1).

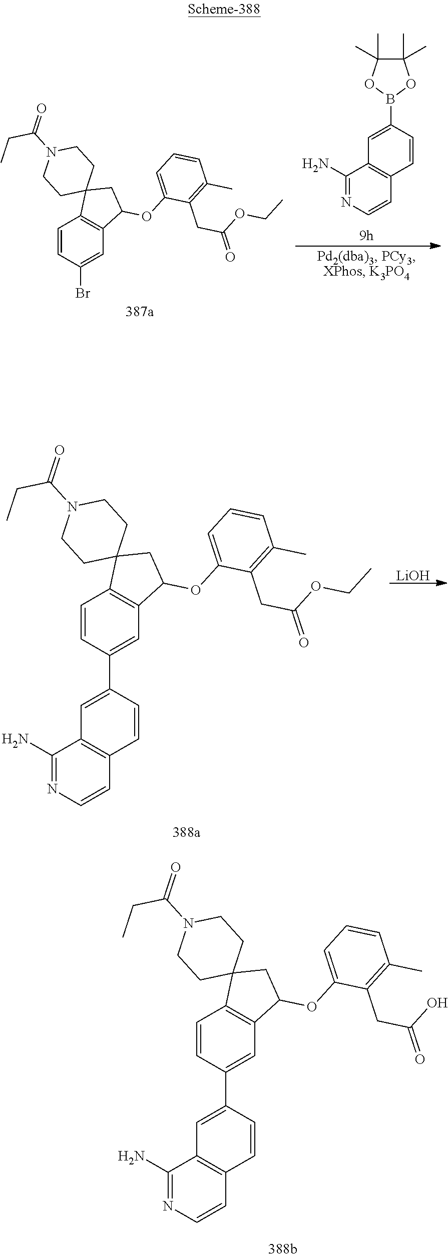

### Preparation of 2-(2-((5-(1-aminoisoquinolin-7-yl)-1′-propionyl-2,3-dihydrospiro[indene-1,4′ piperidin]-3-yl)oxy)-6-methylphenyl)acetic acid (388b)

### Step-1: Preparation of ethyl 2-(2-((5-(1-aminoisoquinolin-7-yl)-1′-propionyl-2,3-dihydrospiro[indene-1,4′-piperidin]-3-yl)oxy)-6-methylphenyl)acetate (388a)

Compound 388a was prepared according to the procedure reported in step-3 of scheme-112, from ethyl 2-(2-((5-bromo-1′-propionyl-2,3-dihydrospiro[indene-1,4′-piperidin]-3-yl)oxy)-6-methylphenyl)acetate (387a) (220 mg, 0.428 mmol) in dioxane/2-MeTHF (2 mL) using 7-(4,4,5,5-tetramethyl-1,3,2-dioxaborolan-2-yl)isoquinolin-1-amine (9h) (231 mg, 0.855 mmol), K₃PO₄ (3 M aqueous solution, 285 μl, 0.855 mmol), PCy₃ (48.0 mg, 0.171 mmol), XPhos (20.39 mg, 0.043 mmol), Pd₂(dba)₃ (78 mg, 0.086 mmol) and heating at 100° C. for 10 h to afford after workup and purification using method-F followed by method-BV, ethyl 2-(2-((5-(1-aminoisoquinolin-7-yl)-1′-propionyl-2,3-dihydrospiro[indene-1,4′-piperidin]-3-yl)oxy)-6-methylphenyl)acetate (388a) (50 mg, 20% yield) as a white solid; MS (ES+): 578.3 (M+1).

### Step-2: Preparation of 2-(2-((5-(1-aminoisoquinolin-7-yl)-1′-propionyl-2,3-dihydrospiro[indene-1,4′-piperidin]-3-yl)oxy)-6-methylphenyl)acetic acid (388b)

Compound 388b was prepared according to the procedure reported in step-2 of scheme-1, from ethyl 2-(2-((5-(1-aminoisoquinolin-7-yl)-1′-propionyl-2,3-dihydrospiro[indene-1,4′-piperidin]-3-yl)oxy)-6-methylphenyl)acetate (388a) (40 mg, 0.069 mmol) in MeOH (1 mL) and THF (1 mL) using lithium hydroxide (1 N solution, 346 μl, 0.346 mmol) to afford after work up and purification using method-BV, 2-(2-((5-(1-aminoisoquinolin-7-yl)-1′-propionyl-2,3-dihydrospiro[indene-1,4′-piperidin]-3-yl)oxy)-6-methylphenyl)acetic acid (388b) (25 mg, 66% yield) HCl salt as a white solid; ¹H NMR (300 MHz, DMSO-d₆) δ 13.22 (s, 1H, D₂O exchangeable), 12.01 (s, 1H, D₂O exchangeable), 9.10 (s, 2H, D₂O exchangeable), 8.89 (s, 1H), 8.31 (dd, J=8.5, 1.6 Hz, 1H), 8.04 (d, J=8.5 Hz, 1H), 7.96-7.83 (m, 2H), 7.69 (d, J=6.9 Hz, 1H), 7.55 (d, J=8.0 Hz, 1H), 7.32-7.12 (m, 3H), 6.83 (dd, J=6.8, 1.9 Hz, 1H), 5.93 (dd, J=6.8, 4.0 Hz, 1H), 4.46 (t, J=12.5 Hz, 1H), 3.88 (t, J=16.0 Hz, 1H), 3.61-3.41 (m, 2H), 3.28-3.02 (m, 1H), 2.88-2.57 (m, 2H), 2.44-2.29 (m, 2H), 2.22 (s, 3H), 2.18-2.02 (m, 1H), 2.01-1.40 (m, 4H), 1.03 (t, J=7.4 Hz, 3H); MS (ES+): 550.3 (M+1); (ES−): 548.3 (M−1).

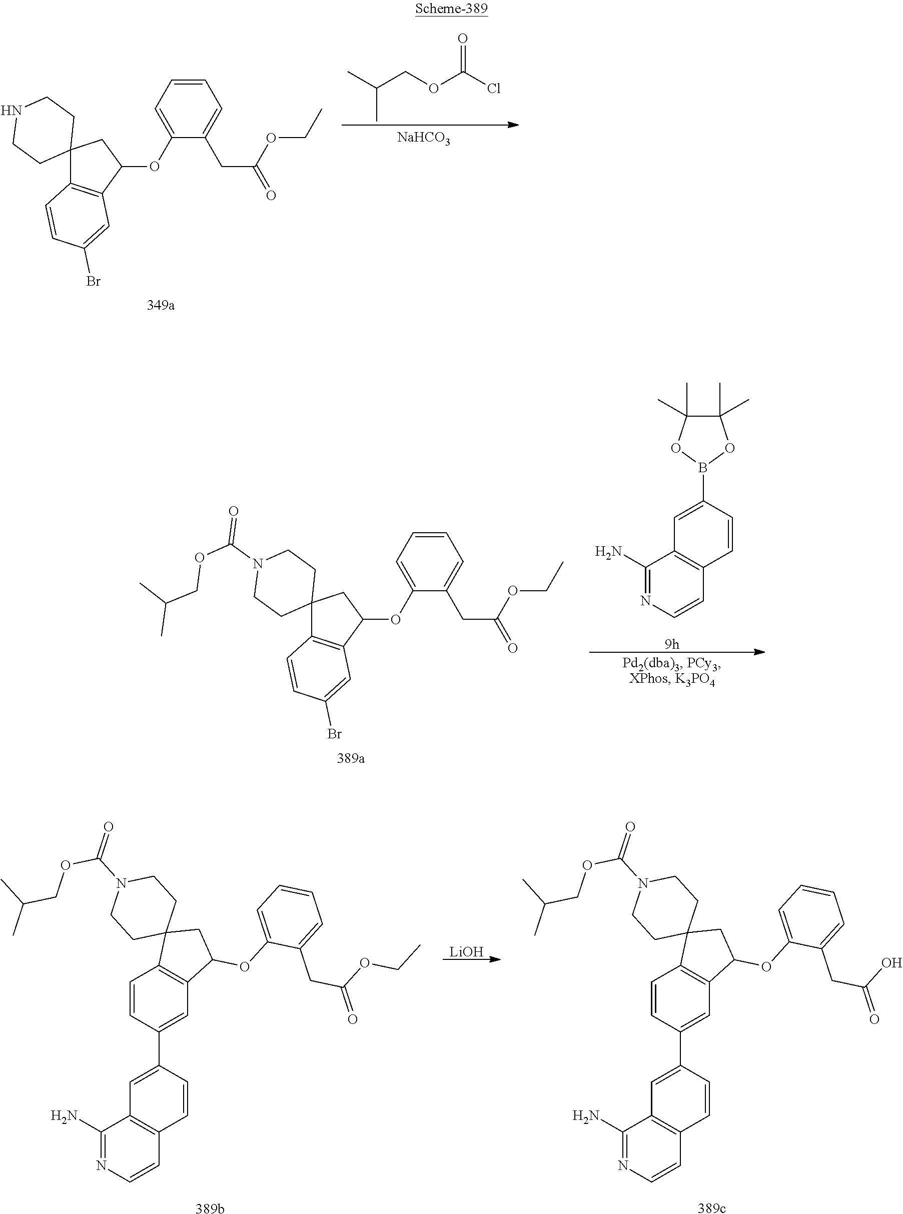

### Preparation of 2-(2-((5-(1-aminoisoquinolin-7-yl)-1′-(isobutoxycarbonyl)-2,3-dihydrospiro[indene-1,4′-piperidin]-3-yl)oxy)phenyl)acetic acid (389c)

### Step-1: Preparation of isobutyl 5-bromo-3-(2-(2-ethoxy-2-oxoethyl)phenoxy)-2,3-dihydrospiro[indene-1,4′-piperidine]-1′-carboxylate (389a)

Compound 389a was prepared according to the procedure reported in step-1 of scheme-126, from ethyl 2-(2-((5-bromo-2,3-dihydrospiro[indene-1,4′-piperidin]-3-yl)oxy)phenyl)acetate, TFA (349a) (0.56 g, 1.003 mmol) in THF (10 mL) using sodium bicarbonate (2.507 mL, 5.01 mmol) and isobutyl carbonochloridate (0.205 g, 1.504 mmol) to afford after workup and purification using method-P, isobutyl 5-bromo-3-(2-(2-ethoxy-2-oxoethyl)phenoxy)-2,3-dihydrospiro[indene-1,4′-piperidine]-1′-carboxylate (389a) (0.49 g, 90% yield) as a clear oil; MS (ES+): 566.1 (M+Na).

### Step-2: Preparation of isobutyl 5-(1-aminoisoquinolin-7-yl)-3-(2-(2-ethoxy-2-oxoethyl)phenoxy)-2,3-dihydrospiro[indene-1,4′-piperidine]-1′-carboxylate (389b)

Compound 389b was prepared according to the procedure reported in step-3 of scheme-112, from isobutyl 5-bromo-3-(2-(2-ethoxy-2-oxoethyl)phenoxy)-2,3-dihydrospiro[indene-1,4′-piperidine]-1′-carboxylate (389a) (220 mg, 0.404 mmol) in dioxane/2-MeTHF (2 mL) using 7-(4,4,5,5-tetramethyl-1,3,2-dioxaborolan-2-yl)isoquinolin-1-amine (9h) (218 mg, 0.808 mmol), K₃PO₄ (3 M aqueous solution, 269 μl, 0.808 mmol), PCy₃ (45.3 mg, 0.162 mmol), XPhos (19.26 mg, 0.040 mmol), Pd₂(dba)₃ (74.0 mg, 0.081 mmol) and heating at 100° C. for 10 h to afford after workup and purification using method-F followed by method-BV, isobutyl 5-(1-aminoisoquinolin-7-yl)-3-(2-(2-ethoxy-2-oxoethyl)phenoxy)-2,3-dihydrospiro[indene-1,4′-piperidine]-1′-carboxylate (389b) (65 mg, 27% yield) as a white solid; MS (ES+): 608.3 (M+1).

### Step-3: Preparation of 2-(2-((5-(1-aminoisoquinolin-7-yl)-1′-(isobutoxycarbonyl)-2,3-dihydrospiro [indene-1,4′-piperidin]-3-yl)oxy)phenyl)acetic acid (389c)

Compound 389c was prepared according to the procedure reported in step-2 of scheme-1, from isobutyl 5-(1-aminoisoquinolin-7-yl)-3-(2-(2-ethoxy-2-oxoethyl)phenoxy)-2,3-dihydrospiro[indene-1,4′-piperidine]-1′-carboxylate (389b) (60 mg, 0.099 mmol) in MeOH (0.6 mL) and THF (0.6 mL) using lithium hydroxide (1N solution, 296 μl, 0.296 mmol) to afford after work up and purification using method-BV, 2-(2-((5-(1-aminoisoquinolin-7-yl)-1′-(isobutoxycarbonyl)-2,3-dihydrospiro[indene-1,4′-piperidin]-3-yl)oxy)phenyl)acetic acid (389c) (15 mg, 26% yield) HCl salt as an off white solid; ¹H NMR (300 MHz, DMSO-d₆) δ 13.11 (s, 1H, D₂O exchangeable), 12.07 (s, 1H, D₂O exchangeable), 9.04 (s, 2H, D₂O exchangeable), 8.88 (s, 1H), 8.31 (dd, J=8.5, 1.6 Hz, 1H), 8.04 (d, J=8.5 Hz, 1H), 7.95-7.82 (m, 2H), 7.69 (d, J=6.9 Hz, 1H), 7.57 (d, J=8.0 Hz, 1H), 7.36-7.17 (m, 4H), 6.98-6.85 (m, 1H), 6.01-5.86 (m, 1H), 4.04 (t, J=12.0 Hz, 2H), 3.82 (d, J=6.5 Hz, 2H), 3.47 (d, J=3.2 Hz, 2H), 3.19-2.82 (m, 2H), 2.82-2.66 (m, 1H), 2.09 (dd, J=13.6, 4.2 Hz, 1H), 1.99-1.72 (m, 3H), 1.70-1.45 (m, 2H), 0.92 (d, J=6.7 Hz, 6H); MS (ES+): 580.3 (M+1); (ES−): 578.2 (M−1).

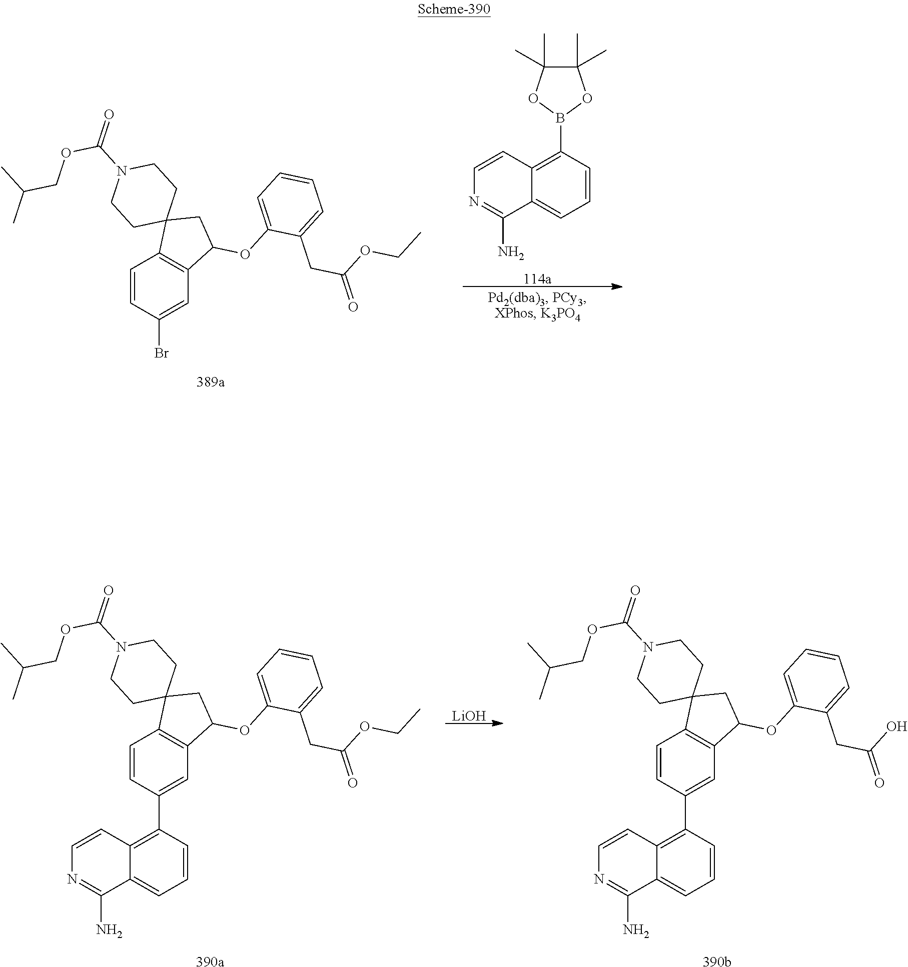

### Preparation of 2-(2-((5-(1-aminoisoquinolin-5-yl)-1′-(isobutoxycarbonyl)-2,3-dihydrospiro[indene-1,4′-piperidin]-3-yl)oxy)phenyl)acetic acid (390b)

### Step-1: Preparation of isobutyl 5-(1-aminoisoquinolin-5-yl)-3-(2-(2-ethoxy-2-oxoethyl)phenoxy)-2,3-dihydrospiro[indene-1,4′-piperidine]-1′-carboxylate (390a)

Compound 390a was prepared according to the procedure reported in step-3 of scheme-112, from isobutyl 5-bromo-3-(2-(2-ethoxy-2-oxoethyl)phenoxy)-2,3-dihydrospiro[indene-1,4′-piperidine]-1′-carboxylate (389a) (220 mg, 0.404 mmol) in dioxane/2-MeTHF (2 mL) using 5-(4,4,5,5-tetramethyl-1,3,2-dioxaborolan-2-yl)isoquinolin-1-amine (114a) (218 mg, 0.808 mmol), K₃PO₄ (3M aqueous solution, 269 μl, 0.808 mmol). PCy₃ (45.3 mg, 0.162 mmol), XPhos (19.26 mg, 0.04 mmol) and Pd₂(dba)₃ (74 mg, 0.081 mmol) and heating at 100° C. for 10 h to afford after workup and purification using method-F followed by method-BV, isobutyl 5-(1-aminoisoquinolin-5-yl)-3-(2-(2-ethoxy-2-oxoethyl)phenoxy)-2,3-dihydrospiro[indene-1,4′-piperidine]-1′-carboxylate (390a) (140 mg, 57% yield) as a white solid; MS (ES+): 608.3 (M+1).

### Step-2: Preparation of 2-(2-((5-(1-aminoisoquinolin-5-yl)-1′-(isobutoxycarbonyl)-2,3-dihydrospiro[indene-1,4′-piperidin]-3-yl)oxy)phenyl)acetic acid (390b)

Compound 390b was prepared according to the procedure reported in step-2 of scheme-1, from isobutyl 5-(1-aminoisoquinolin-5-yl)-3-(2-(2-ethoxy-2-oxoethyl)phenoxy)-2,3-dihydrospiro[indene-1,4′-piperidine]-1′-carboxylate (390a) (120 mg, 0.197 mmol) in MeOH (2 mL) and THF (2 mL) using lithium hydroxide (1N solution, 987 μl, 0.987 mmol) to afford after work up and purification using method-BV, 2-(2-((5-(1-aminoisoquinolin-5-yl)-1′-(isobutoxycarbonyl)-2,3-dihydrospiro[indene-1,4′-piperidin]-3-yl)oxy)phenyl)acetic acid (390b) (54 mg, 47% yield) HCl salt as a white solid; ¹H NMR (300 MHz, DMSO-d₆) δ 13.03 (s, 1H, D₂O exchangeable), 11.97 (s, 1H, D₂O exchangeable), 8.97 (s, 2H, D₂O exchangeable), 8.57 (d, J=8.3 Hz, 1H), 7.94-7.77 (m, 2H), 7.60 (d, J=7.2 Hz, 1H), 7.55 (d, J=7.8 Hz, 1H), 7.47-7.37 (m, 2H), 7.34-7.22 (m, 2H), 7.19 (d, J=7.4 Hz, 1H), 7.00-6.86 (m, 2H), 5.94 (t, J=5.6 Hz, 1H), 4.19-3.93 (m, 2H), 3.82 (d, J=6.5 Hz, 2H), 3.44 (s, 2H), 3.21-2.88 (m, 2H), 2.81 (dd, J=13.6, 6.8 Hz, 1H), 2.10 (dd, J=13.6, 4.5 Hz, 1H), 2.01-1.75 (m, 3H), 1.72-1.53 (m, 2H), 0.92 (d, J=6.7 Hz, 6H); MS (ES+):580.3 (M+1); (ES−): 578.2 (M−1).

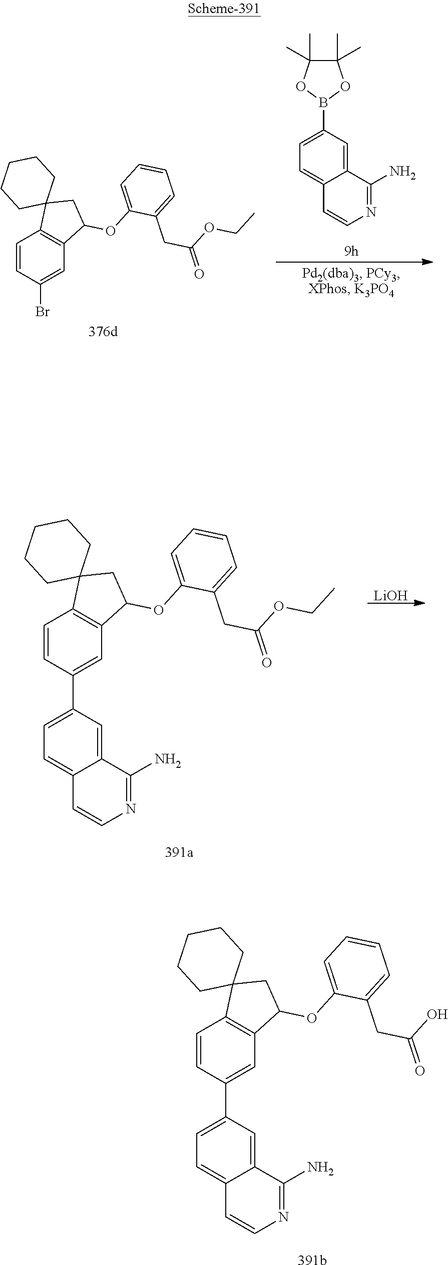

### Preparation of 2-(2-((5′-(1-aminoisoquinolin-7-yl)-2′,3′-dihydrospiro[cyclohexane-1,1′-inden]-3′-yl)oxy)phenyl)acetic acid (391b)

### Step-1: Preparation of ethyl 2-(2-((5′-(1-aminoisoquinolin-7-yl)-2′,3′-dihydrospiro[cyclohexane-1,1′-inden]-3′-yl)oxy)phenyl)acetate (391a)

Compound 391a was prepared according to the procedure reported in step-3 of scheme-112, from ethyl 2-(2-((5′-bromo-2′,3′-dihydrospiro[cyclohexane-1,1′-inden]-3′-yl)oxy)phenyl)acetate (376d) (120 mg, 0.271 mmol) in dioxane/2-MeTHF (2 mL) using 7-(4,4,5,5-tetramethyl-1,3,2-dioxaborolan-2-yl)isoquinolin-1-amine (9h) (146 mg, 0.541 mmol), K₃PO₄ (3M aqueous solution, 180 μl, 0.541 mmol), PCy₃ (30.4 mg, 0.108 mmol), XPhos (25.8 mg, 0.054 mmol), Pd₂(dba)₃ (49.6 mg, 0.054 mmol) and heating at 100° C. for 10 h to afford after workup and purification using method-U, ethyl 2-(2-((5′-(1-aminoisoquinolin-7-yl)-2′,3′-dihydrospiro[cyclohexane-1,1′-inden]-3′-yl)oxy)phenyl)acetate (391a) (62 mg, 45% yield) as a clear oil; MS (ES+): 507.3 (M+1); (ES−): 505.2 (M−1).

### Step-2: Preparation of 2-(2-((5′-(1-aminoisoquinolin-7-yl)-2′,3′-dihydrospiro[cyclohexane-1,1′-inden]-3′-yl)oxy)phenyl)acetic acid (391b)

Compound 391b was prepared according to the procedure reported in step-2 of scheme-1, from ethyl 2-(2-((5′-(1-aminoisoquinolin-7-yl)-2′,3′-dihydrospiro[cyclohexane-1,1′-inden]-3′-yl)oxy)phenyl)acetate (391a) (62 mg, 0.122 mmol) in THF (3 mL) using lithium hydroxide (19.44 mg, 0.812 mmol) in water (1 mL) to afford after work up and purification using method-M, 2-(2-((5′-(1-aminoisoquinolin-7-yl)-2′,3′-dihydrospiro[cyclohexane-1,1′-inden]-3′-yl)oxy)phenyl)acetic acid (391b) (28 mg, 22% yield) HCl salt as a white solid; ¹H NMR (300 MHz, DMSO-d₆) δ 12.76 (s, 1H, D₂O exchangeable), 9.16 (s, 2H, D₂O exchangeable), 8.90 (d, J=1.7 Hz, 1H), 8.29 (dd, J=8.5, 1.6 Hz, 1H), 8.02 (d, J=8.5 Hz, 1H), 7.90 (dd, J=8.0, 1.8 Hz, 1H), 7.86-7.81 (m, 1H), 7.70 (d, J=6.9 Hz, 1H), 7.51 (d, J=8.0 Hz, 1H), 7.33-7.17 (m, 4H), 6.98-6.86 (m, 1H), 5.96-5.80 (m, 1H), 3.48 (d, J=3.0 Hz, 2H), 2.68 (dd, J=13.7, 7.1 Hz, 1H), 1.96 (dd, J=13.7, 4.5 Hz, 1H), 1.78-1.49 (m, 8H), 1.42-1.22 (m, 2H); MS (ES+): 479.2 (M+1); (ES−): 477.3 (M−1).

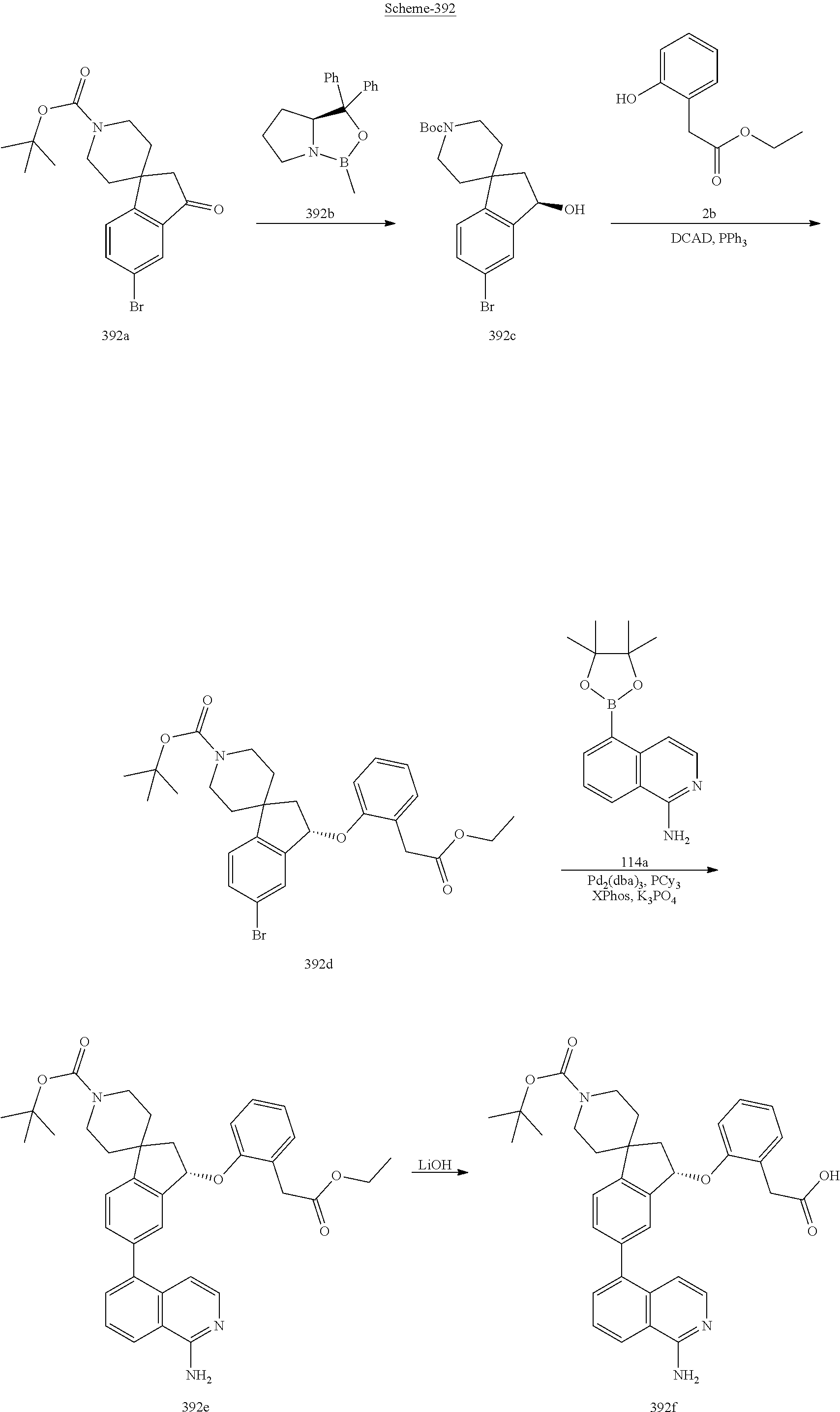

### Preparation of (S)-2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (392f)

### Step-1: Preparation of tert-butyl (R)-5-bromo-3-hydroxy-2,3-dihydrospiro[indene-1,4′-piperidine]-1′-carboxylate (392c)

Compound 392c was prepared according to the procedure reported by Nakamura, Yuji; Jojima, Takaaki; Suzuki, Chie; Miyazaki, Shojiro; and Nishi, Takahide in Synlett (2009). (15), 2521-2523, as follows. To a solution of (S)-2-Methyl-CBS-oxazaborolidine (392b) (0.146 g, 0.526 mmol; CAS #: 112022-81-8) in anhydrous THF (10 mL) at RT was added tert-butyl 5-bromo-3-oxo-2,3-dihydrospiro[indene-1,4′-piperidine]-1′-carboxylate (392a) (2 g, 5.26 mmol; CAS #1160247-30-2) in THF (2 mL) followed by BH₃. THF (1.0 M solution in THF; 3.16 mL, 3.16 mmol) and stirred for 30 min at RT. The reaction was quenched by cold MeOH and water (10 mL, 1:4, v/v) and extracted with EtOAc (3×). Combined organics were washed with brine, dried, filtered and concentrated in vacuo. The residue obtained was purified using method-J to afford tert-butyl (R)-5-bromo-3-hydroxy-2,3-dihydrospiro[indene-1,4′-piperidine]-1′-carboxylate (392c) (1.2 g, 60% yield) as a white solid; ¹H NMR (300 MHz, DMSO-d₆) δ 7.46 (d, J=1.9 Hz, 1H), 7.42 (dd, J=8.1, 2.0 Hz, 1H), 7.23 (d, J=8.1 Hz, 1H), 5.46-5.33 (m, 1H), 5.17-5.04 (m, 1H), 4.07-3.86 (m, 2H), 3.06-2.80 (m, 2H), 1.93-1.78 (m, 1H), 1.77-1.65 (m, 1H), 1.60-1.46 (m, 2H), 1.42 (s, 9H), 1.41-1.29 (m, 2H); [α]_{D}=−12.8 [MeOH, 0.25].

### Step-2: Preparation of (S)-tert-butyl 5-bromo-3-(2-(2-ethoxy-2-oxoethyl)phenoxy)-2,3-dihydrospiro[indene-1,4′-piperidine]-1′-carboxylate (392d)

Compound 392d was prepared according to the procedure reported in step-2 of scheme-2, from tert-butyl (R)-5-bromo-3-hydroxy-2,3-dihydrospiro[indene-1,4′-piperidine]-1′-carboxylate (392c) (1.18 g, 3.09 mmol) in DCM (30 mL) using PPh₃ (0.891 g, 3.40 mmol), ethyl 2-(2-hydroxyphenyl)acetate (2b) (0.612 g, 3.40 mmol) and a solution of DCAD (1.247 g, 3.40 mmol) in DCM (60 mL) to afford after workup and purification using method-V. (S)-tert-butyl 5-bromo-3-(2-(2-ethoxy-2-oxoethyl)phenoxy)-2,3-dihydrospiro[indene-1,4′-piperidine]-1′-carboxylate (392d) (758 mg, 45% yield) as a clear oil; MS (ES+): 566.2 and 568.2 (M+Na); (ES−) 542.1 and 544.1 (M−1); [α]_{D}=+53.585 [MeOH, 0.265].

### Step-3: Preparation of (S)-tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (392e)

Compound 392e was prepared according to the procedure reported in step-3 of scheme-112, from (S)-tert-butyl 5-bromo-3-(2-(2-ethoxy-2-oxoethyl)phenoxy)-2,3-dihydrospiro[indene-1,4′-piperidine]-1′-carboxylate (392d) (100 mg, 0.184 mmol) in dioxane/2-MeTHF (2 mL) using 5-(4,4,5,5-tetramethyl-1,3,2-dioxaborolan-2-yl)isoquinolin-1-amine (114a) (99 mg, 0.367 mmol), K₃PO₄ (3M aqueous solution, 122 μl, 0.367 mmol), PCy₃ (20.60 mg, 0.073 mmol), XPhos (17.51 mg, 0.037 mmol), Pd₂(dba)₃ (33.6 mg, 0.037 mmol) and heating at 100° C. for 10 h to afford after workup and purification using method-U. (S)-tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (392e) (76 mg, 68% yield) as a clear oil; MS (ES+): 608.3 (M+1); (ES−): 606.3 (M−1).

### Step-4: Preparation of (S)-2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (392f)

Compound 392f was prepared according to the procedure reported in step-2 of scheme-1, from (S)-tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (392e) (76 mg, 0.125 mmol) in THF (3 mL) using lithium hydroxide (13.19 mg, 0.551 mmol) in water (1 mL) to afford after work up and purification using method-M, (S)-2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (392f) (49 mg, 46% yield) HCl salt as a white solid; ¹H NMR (300 MHz, DMSO-d₆) δ 13.32 (s, 1H, D₂O exchangeable), 12.04 (s, 1H, D₂O exchangeable), 9.14 (s, 2H, D₂O exchangeable), 8.61 (d, J=8.2 Hz, 1H), 7.94-7.87 (m, 1H), 7.83 (t, J=7.8 Hz, 1H), 7.62 (d, J=7.2 Hz, 1H), 7.54 (d, J=7.8 Hz, 1H), 7.48-7.38 (m, 2H), 7.33-7.24 (m, 2H), 7.22-7.14 (m, 1H), 7.00-6.87 (m, 2H), 5.98-5.88 (m, 1H), 4.11-3.87 (m, 2H), 3.44 (s, 2H), 3.12-2.72 (m, 3H), 2.14-2.01 (m, 1H), 1.97-1.86 (m, 1H), 1.83-1.72 (m, 1H), 1.71-1.62 (m, 1H), 1.62-1.53 (m, 1H), 1.43 (s, 9H); MS (ES+): 580.3 (M+1); (ES−): 578.3 (M−1); [α]_{D}=+21.176 [MeOH, 0.255]

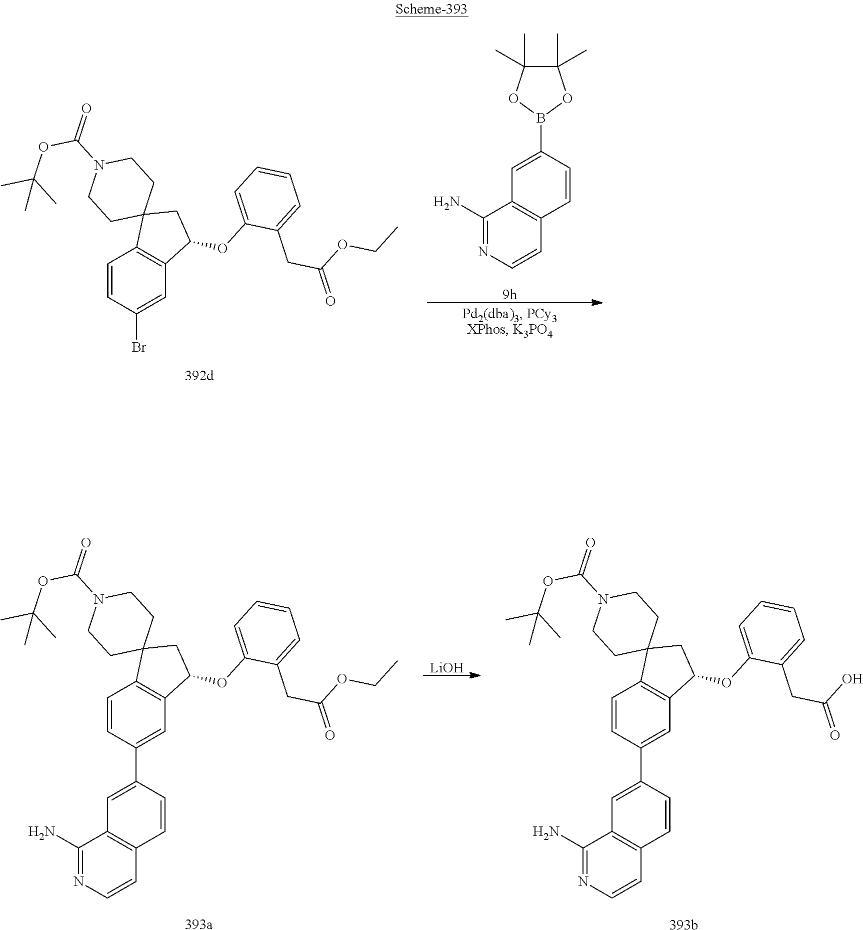

### Preparation of (S)-2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (393b)

### Step-1: Preparation of (S)-tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (393a)

Compound 393a was prepared according to the procedure reported in step-3 of scheme-112, from (S)-tert-butyl 5-bromo-3-(2-(2-ethoxy-2-oxoethyl)phenoxy)-2,3-dihydrospiro[indene-1,4′-piperidine]-1′-carboxylate (392d) (100 mg, 0.184 mmol) in dioxane/2-MeTHF (2 mL) using 7-(4,4,5,5-tetramethyl-1,3,2-dioxaborolan-2-yl)isoquinolin-1-amine (9h) (99 mg, 0.367 mmol), K₃PO₄ (3M aqueous solution, 122 μl, 0.367 mmol), PCy₃ (21 mg, 0.073 mmol), XPhos (9 mg, 0.018 mmol). Pd₂(dba)₃ (34 mg, 0.037 mmol) and heating at 100° C. for 10 h to afford after workup and purification using method-U, (S)-tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (393a) (66 mg, 59% yield) as a clear oil; MS (ES+): 608.4 (M+1); (ES−): 606.3 (M−1).

### Step-2: Preparation of (S)-2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (393b)

Compound 393b was prepared according to the procedure reported in step-2 of scheme-1, from (S)-tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (393a) (66 mg, 0.109 mmol) in THF (3 mL) using lithium hydroxide (13.19 mg, 0.551 mmol) in water (1 mL) to afford after work up and purification using method-M, (S)-2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (393b) (33 mg, 31% yield) HCl salt as a white solid; ¹H NMR (300 MHz, DMSO-d₆) δ 13.29 (s, 1H, D₂O exchangeable), 12.12 (s, 1H, D₂O exchangeable), 9.14 (s, 2H, D₂O exchangeable), 8.90 (s, 1H), 8.32 (dd, J=8.5, 1.6 Hz, 1H), 8.04 (d, J=8.5 Hz, 1H), 7.96-7.83 (m, 2H), 7.74-7.66 (m, 1H), 7.56 (d, J=8.0 Hz, 1H), 7.35-7.17 (m, 4H), 6.99-6.88 (m, 1H), 6.00-5.88 (m, 1H), 4.10-3.88 (m, 2H), 3.48 (d, J=3.1 Hz, 2H), 3.11-2.81 (m, 2H), 2.81-2.70 (m, 1H), 2.07 (dd, J=13.7, 4.2 Hz, 1H), 1.95-1.71 (m, 2H), 1.68-1.50 (m, 2H), 1.44 (s, 9H); MS (ES+): 580.3 (M+1); (ES−): 578.3 (M−1); [α]_{D}=+80.0 [MeOH, 0.255].

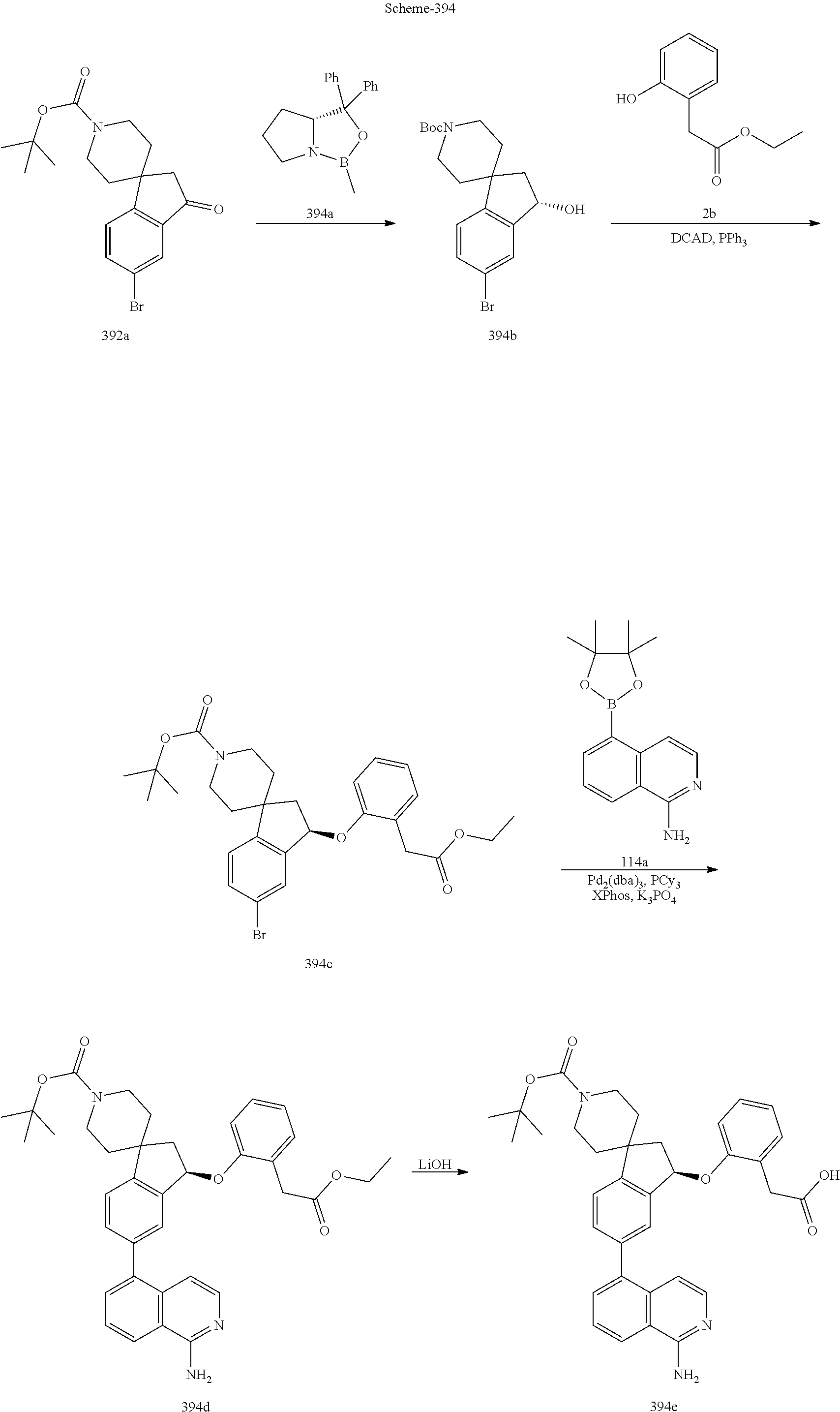

### Preparation of (R)-2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (394e)

### Step-1: Preparation of (S)-tert-butyl 5-bromo-3-hydroxy-2,3-dihydrospiro[indene-1,4′-piperidine]-1′-carboxylate (394b)

Compound 394b was prepared according to the procedure reported by Nakamura, Yuji; Jojima, Takaaki; Suzuki, Chie; Miyazaki, Shojiro; and Nishi, Takahide in Synlett (2009), (15), 2521-2523, as described in step-1 of scheme-392, from tert-butyl 5-bromo-3-oxo-2,3-dihydrospiro[indene-1,4′-piperidine]-1′-carboxylate (392a) (2 g, 5.26 mmol) in THF (2 mL) using (R)-2-Methyl-CBS-oxazaborolidine (394a) (1.458 g, 5.26 mmol; CAS #: 112022-83-0) in anhydrous THF (20 mL) and BH₃. THF (1.0 M solution in THF; 3.16 mL, 3.16 mmol) to afford after work up and purification using method-J, (S)-tert-butyl 5-bromo-3-hydroxy-2,3-dihydrospiro[indene-1,4′-piperidine]-1′-carboxylate (394b) (1.3 g, 65% yield) as a white solid; ¹H NMR (300 MHz, DMSO-d₆) δ 7.50-7.36 (m, 2H), 7.21 (dd. J=8.1, 2.2 Hz, 1H), 5.48-5.32 (m, 1H), 5.14-5.00 (m, 1H), 4.05-3.80 (m, 2H), 3.05-2.72 (m, 2H), 1.91-1.76 (m, 1H), 1.75-1.64 (m, 1H), 1.55-1.31 (m, 13H); [α]_{D}=+13.091 [MeOH, 0.275].

### Step-2: Preparation of (R)-tert-butyl 5-bromo-3-(2-(2-ethoxy-2-oxoethyl)phenoxy)-2,3-dihydrospiro[indene-1,4′-piperidine]-1′-carboxylate (394c)

Compound 394c was prepared according to the procedure reported in step-2 of scheme-2, from (S)-tert-butyl 5-bromo-3-hydroxy-2,3-dihydrospiro[indene-1,4′-piperidine]-1′-carboxylate (394b) (1.1 g, 2.88 mmol) in DCM (30 mL) using PPh₃ (0.830 g, 3.17 mmol), ethyl 2-(2-hydroxyphenyl)acetate (2b) (0.570 g, 3.17 mmol) and a solution of DCAD (1.162 g, 3.17 mmol) in DCM (60 mL) to afford after workup and purification using method-V. (R)-tert-butyl 5-bromo-3-(2-(2-ethoxy-2-oxoethyl)phenoxy)-2,3-dihydrospiro[indene-1,4′-piperidine]-1′-carboxylate (394c) (601 mg, 1.104 mmol, 38.4% yield) as a white solid; MS (ES+): 566.2 and 568.1 (M+Na); (ES−): 542.1 and 544.1 (M−1); [α]_{D}=−62.857 [MeOH, 0.245].

### Step-3: Preparation of (R)-tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (394d)

Compound 394d was prepared according to the procedure reported in step-3 of scheme-112, from (R)-tert-butyl 5-bromo-3-(2-(2-ethoxy-2-oxoethyl)phenoxy)-2,3-dihydrospiro[indene-1,4′-piperidine]-1′-carboxylate (394c) (100 mg, 0.184 mmol) in dioxane/2-MeTHF (2 mL) using 5-(4,4,5,5-tetramethyl-1,3,2-dioxaborolan-2-yl)isoquinolin-1-amine (114a) (99 mg, 0.367 mmol), K₃PO₄ (3 M aqueous solution, 122 μl, 0.367 mmol), PCy₃ (21 mg, 0.073 mmol). XPhos (18 mg, 0.037 mmol) and Pd₂(dba)₃ (34 mg, 0.037 mmol) and heating at 100° C. for 10 h to afford after workup and purification using method-U, (R)-tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (394d) (80 mg, 72% yield) as a clear oil; MS (ES+): 608.3 (M+1); (ES−): 606.3 (M−1).

### Step-4: Preparation of (R)-2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (394e)

Compound 394e was prepared according to the procedure reported in step-2 of scheme-1, from (R)-tert-butyl 5-(1-aminoisoquinolin-5-yl)-3-(2-(2-ethoxy-2-oxoethyl)phenoxy)-2,3-dihydrospiro[indene-1,4′-piperidine]-1′-carboxylate (394d) (80 mg, 0.132 mmol) in THF (3 mL) using lithium hydroxide (13.19 mg, 0.551 mmol) in water (1 mL) to afford after work up and purification using method-M, (R)-2-(2-((5-(1-aminoisoquinolin-5-yl)-1′-(tert-butoxycarbonyl)-2,3-dihydrospiro[indene-1,4′-piperidin]-3-yl)oxy)phenyl)acetic acid (394e) (51 mg, 48% yield) HCl salt as a white solid; ¹H NMR (300 MHz, DMSO-d₆) δ 13.29 (s, 1H, D₂O exchangeable), 12.04 (s, 1H, D₂O exchangeable), 9.13 (s, 2H, DO exchangeable), 8.60 (d, J=8.2 Hz, 1H), 7.91 (dd, J=7.3, 1.2 Hz, 1H), 7.87-7.79 (m, 1H), 7.61 (d, J=7.3 Hz, 1H), 7.57-7.50 (m, 1H), 7.47-7.38 (m, 2H), 7.31-7.24 (m, 2H), 7.22-7.15 (m, 1H), 6.96 (d, J=7.2 Hz, 1H), 6.94-6.86 (m, 1H), 6.06-5.83 (m, 1H), 4.11-3.87 (m, 2H), 3.44 (s, 2H), 3.12-2.72 (m, 3H), 2.15-2.02 (m, 1H), 1.98-1.86 (m, 1H), 1.85-1.72 (m, 1H), 1.72-1.63 (m, 1H), 1.63-1.52 (m, 1H), 1.43 (s, 9H); MS (ES+): 580.3 (M+1); (ES−): 578.3 (M−1); [α]_{D}=−28.462 [MeOH, 0.26].

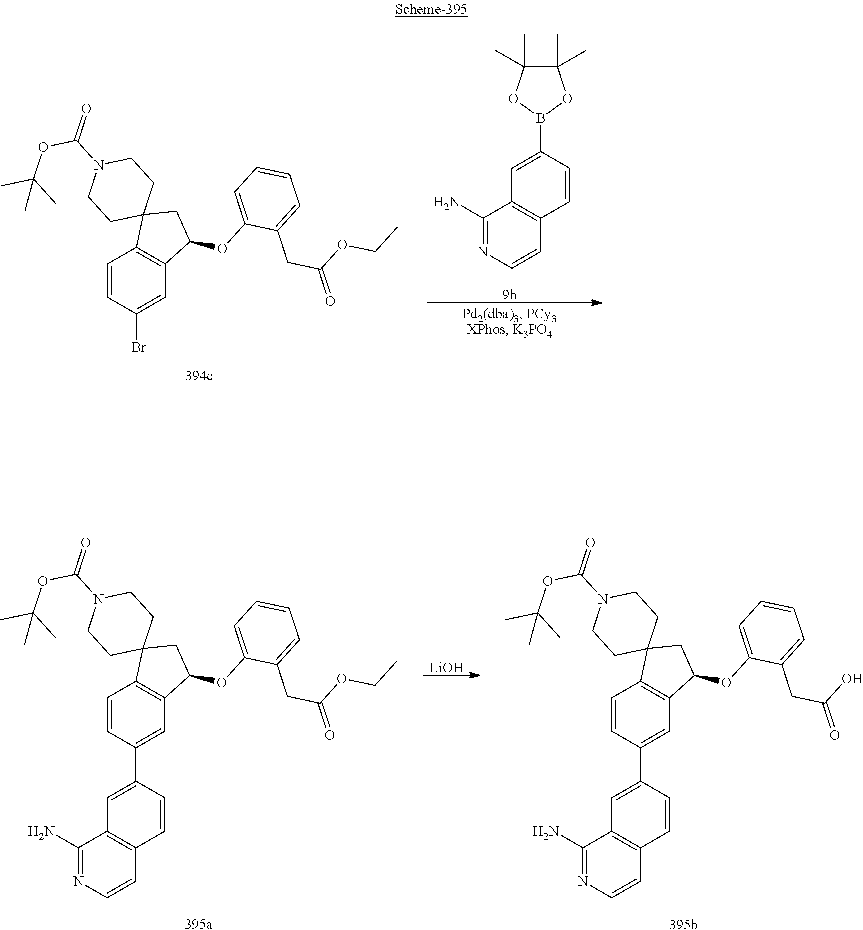

### Preparation of (R)-2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (395b)

### Step-1: Preparation of (R)-tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (395a)

Compound 395a was prepared according to the procedure reported in step-3 of scheme-112, from (R)-tert-butyl 5-bromo-3-(2-(2-ethoxy-2-oxoethyl)phenoxy)-2,3-dihydrospiro[indene-1,4′-piperidine]-1′-carboxylate (394c) (100 mg, 0.184 mmol) in dioxane/2-MeTHF (2 mL) using 7-(4,4,5,5-tetramethyl-1,3,2-dioxaborolan-2-yl)isoquinolin-1-amine (9h) (99 mg, 0.367 mmol), K₃PO₄ (3 M aqueous solution, 122 μl, 0.367 mmol). PCy₃ (21 mg, 0.073 mmol), XPhos (18 mg, 0.037 mmol), Pd₂(dba)₃ (34 mg, 0.037 mmol) and heating at 100° C. for 10 h to afford after workup and purification using method-U, (R)-tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (395a) (63 mg, 56% yield) as a clear oil; MS (ES+): 608.3 (M+1); (ES−): 606.3 (M−1).

### Step-2: Preparation of (R)-2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (395b)

Compound 395b was prepared according to the procedure reported in step-2 of scheme-1, from (R)-tert-butyl 5-(1-aminoisoquinolin-7-yl)-3-(2-(2-ethoxy-2-oxoethyl)phenoxy)-2,3-dihydrospiro[indene-1,4′-piperidine]-1′-carboxylate (395a) (63 mg, 0.104 mmol) in THF (3 mL) using lithium hydroxide (13 mg, 0.551 mmol) in water (1 mL) to afford after work up and purification using method-M, (R)-2-(2-((5-(1-aminoisoquinolin-7-yl)-1′-(tert-butoxycarbonyl)-2,3-dihydrospiro[indene-1,4′-piperidin]-3-yl)oxy)phenyl)acetic acid (395b) (27 mg, 25% yield) HCl salt as a white solid; ¹H NMR (300 MHz, DMSO-d₆) δ 13.27 (s, 1H, D₂O exchangeable), 12.11 (s, 1H, D₂O exchangeable), 9.13 (s, 2H, D₂O exchangeable), 8.89 (d, J=1.7 Hz, 1H), 8.31 (dd, J=8.5, 1.6 Hz, 1H), 8.04 (d, J=8.5 Hz, 1H), 7.94-7.83 (m, 2H), 7.69 (d, J=6.9 Hz, 1H), 7.56 (d, J=8.0 Hz, 1H), 7.36-7.16 (m, 4H), 6.97-6.86 (m, 1H), 5.98-5.89 (m, 1H), 4.11-3.88 (m, 2H), 3.47 (d, J=3.2 Hz, 2H), 3.12-2.81 (m, 2H), 2.80-2.67 (m, 1H), 2.11-2.01 (m, 1H), 1.94-1.72 (m, 2H), 1.66-1.49 (m, 2H), 1.43 (s, 9H); MS (ES+): 580.3 (M+1); (ES−): 578.4 (M−1); [α]_{D}=−98.462 [MeOH, 0.26].

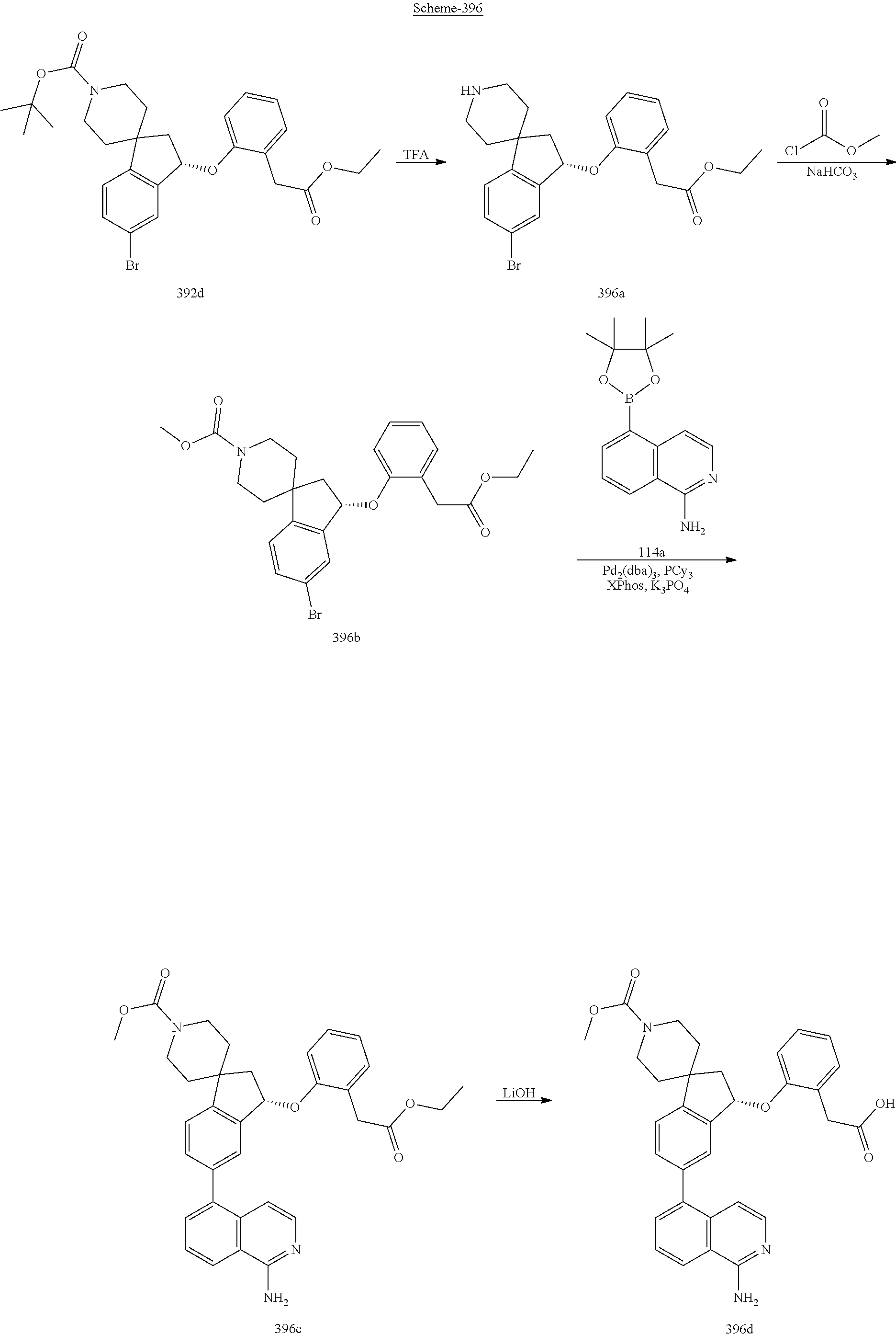

### Preparation of (S)-2-(2-((5-(1-aminoisoquinolin-5-yl)-1′-(methoxycarbonyl)-2,3-dihydrospiro[indene-1,4′-piperidin]-3-yl)oxy)phenyl)acetic acid (396d)

### Step-1: Preparation of (S)-ethyl 2-(2-((5-bromo-2,3-dihydrospiro[indene-1,4′-piperidin]-3-yl)oxy)phenyl)acetate (396a)

Compound 396a was prepared according to the procedure reported in step-4 of scheme-9, from (S)-tert-butyl 5-bromo-3-(2-(2-ethoxy-2-oxoethyl)phenoxy)-2,3-dihydrospiro[indene-1,4′-piperidine]-1′-carboxylate (392d) (500 mg, 0.918 mmol) in DCM (5 mL) using TFA (707 μl, 9.18 mmol) to afford after work up (S)-ethyl 2-(2-((5-bromo-2,3-dihydrospiro[indene-1,4′-piperidin]-3-yl)oxy)phenyl)acetate (396a) (400 mg, 0.900 mmol) and was used as such without further purification; MS (ES+): 444.1 and 446.1 (M+1); (ES−): 442.2 and 444.2 (M−1).

### Step-2: Preparation of (S)-methyl 5-bromo-3-(2-(2-ethoxy-2-oxoethyl)phenoxy)-2,3-dihydrospiro[indene-1,4′-piperidine]-1′-carboxylate (396b)

Compound 396b was prepared according to the procedure reported in step-1 of scheme-126, from (S)-ethyl 2-(2-((5-bromo-2,3-dihydrospiro[indene-1,4′-piperidin]-3-yl)oxy)phenyl)acetate, TFA (396a) (400 mg, 0.716 mmol) in THF (10 mL) using sodium bicarbonate (1791 μl, 3.58 mmol) and methyl carbonochloridate (83 μl, 1.075 mmol) to afford after workup and purification using method-P. (S)-methyl 5-bromo-3-(2-(2-ethoxy-2-oxoethyl)phenoxy)-2,3-dihydrospiro[indene-1,4′-piperidine]-1′-carboxylate (396b) (350 mg, 97% yield) as a clear oil; MS (ES+): 524.2 and 526.1 (M+Na).

### Step-3: Preparation of (S)-methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (396c)

Compound 396c was prepared according to the procedure reported in step-3 of scheme-112, from (S)-methyl 5-bromo-3-(2-(2-ethoxy-2-oxoethyl)phenoxy)-2,3-dihydrospiro[indene-1,4′-piperidine]-1′-carboxylate (396b) (100 mg, 0.199 mmol) in dioxane/2-MeTHF (2 mL) using 5-(4,4,5,5-tetramethyl-1,3,2-dioxaborolan-2-yl)isoquinolin-1-amine (114a) (108 mg, 0.398 mmol), K₃PO₄ (3M aqueous solution, 133 μl, 0.398 mmol), PCy₃ (22 mg, 0.08 mmol), XPhos (19 mg, 0.04 mmol) and Pd₂(dba)₃ (37 mg, 0.04 mmol) and heating at 100° C. for 10 h to afford after workup and purification using method-U, (S)-methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (396c) (60 mg, 53% yield) as a clear oil; MS (ES+): 566.3 (M+1).

### Step-4: Preparation of (S)-2-(2-((5-(1-aminoisoquinolin-5-yl)-1′-(methoxycarbonyl)-2,3-dihydrospiro[indene-1,4′-piperidin]-3-yl)oxy)phenyl)acetic acid (396d)

Compound 396d was prepared according to the procedure reported in step-2 of scheme-1, from (S)-methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (396c) (60 mg, 0.106 mmol) in THF (3 mL) using lithium hydroxide (14.30 mg, 0.597 mmol) in water (1 mL) to afford after work up and purification using method-M, (S)-2-(2-((5-(1-aminoisoquinolin-5-yl)-1′-(methoxycarbonyl)-2,3-dihydrospiro[indene-1,4′-piperidin]-3-yl)oxy)phenyl)acetic acid (396d) (35 mg, 33% yield) HCl salt as a white solid; ¹H NMR (300 MHz, DMSO-d₆) δ13.27 (s, 1H, D₂O exchangeable), 12.00 (s, 1H, D₂O exchangeable), 9.14 (s, 2H, D₂O exchangeable), 8.60 (d, J=8.2 Hz, 1H), 7.95-7.88 (m, 1H), 7.88-7.78 (m, 1H), 7.61 (d, J=7.3 Hz, 1H), 7.55 (d, J=7.8 Hz, 1H), 7.45-7.38 (m, 2H), 7.32-7.24 (m, 2H), 7.22-7.14 (m, 1H), 7.00-6.93 (m, 1H), 6.93-6.85 (m, 1H), 5.94 (dd, J=6.8, 4.5 Hz, 1H), 4.14-3.92 (m, 2H), 3.63 (s, 3H), 3.44 (d, J=1.4 Hz, 2H), 3.18-2.88 (m, 2H), 2.80 (dd, J=13.7, 6.9 Hz, 1H), 2.10 (dd, J=13.7, 4.5 Hz, 1H), 2.00-1.74 (m, 2H), 1.74-1.49 (m, 2H); MS (ES+) 538.2 (M+1); (ES−) 536.3 (M−1); [α]_{D}=+50.4 [MeOH, 0.25].

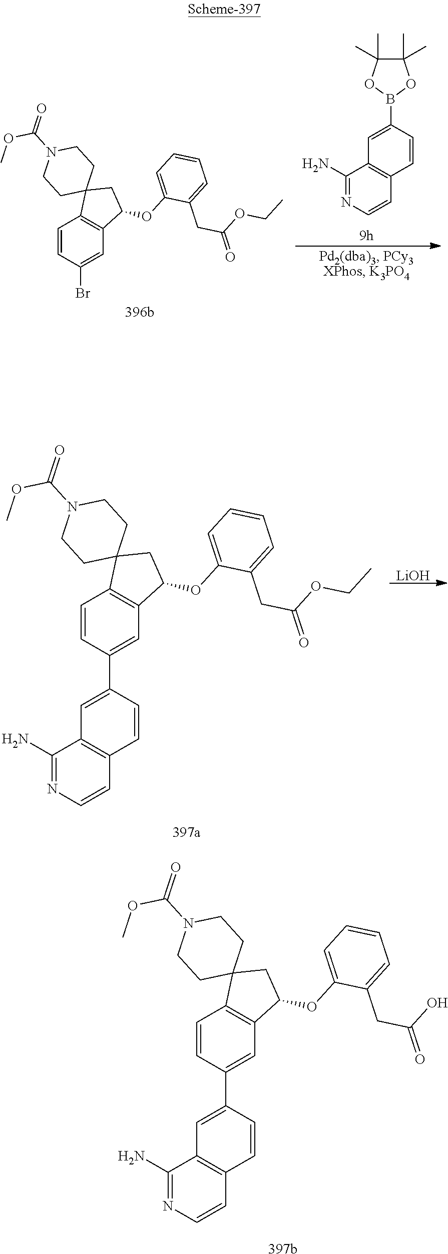

### Preparation of (S)-2-(2-((5-(1-aminoisoquinolin-7-yl)-1′-(methoxycarbonyl)-2,3-dihydrospiro[indene-1,4′-piperidin]-3-yl)oxy)phenyl)acetic acid (397b)

### Step-1: Preparation of (S)-methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (397a)

Compound 397a was prepared according to the procedure reported in step-3 of scheme-112, from (S)-methyl 5-bromo-3-(2-(2-ethoxy-2-oxoethyl)phenoxy)-2,3-dihydrospiro[indene-1,4′-piperidine]-1′-carboxylate (396b) (100 mg, 0.2 mmol) in dioxane/2-MeTHF (2 mL) using 7-(4,4,5,5-tetramethyl-1,3,2-dioxaborolan-2-yl)isoquinolin-1-amine (9h) (108 mg, 0.398 mmol), K₃PO₄ (3 M aqueous solution, 133 μl, 0.398 mmol), PCy₃ (22 mg, 0.08 mmol), XPhos (10 mg, 0.02 mmol), Pd₂(dba)₃ (36.5 mg, 0.04 mmol) and heating at 100° C. for 10 h to afford after workup and purification using method-U. (S)-methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (397a) (63 mg, 56% yield) as a clear oil; MS (ES+): 566.3 (M+1); (ES−): 564.2 (M−1).

### Step-2: Preparation of (S)-2-(2-((5-(1-aminoisoquinolin-7-yl)-1′-(methoxycarbonyl)-2,3-dihydrospiro[indene-1,4′-piperidin]-3-yl)oxy)phenyl)acetic acid (397b)

Compound 397b was prepared according to the procedure reported in step-2 of scheme-1, from (S)-methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (397a) (63 mg, 0.111 mmol) in THF (3 mL) using lithium hydroxide (14.30 mg, 0.597 mmol) in water (1 mL) to afford after work up and purification using method-M, (S)-2-(2-((5-(1-aminoisoquinolin-7-yl)-1′-(methoxycarbonyl)-2,3-dihydrospiro[indene-1,4′-piperidin]-3-yl)oxy)phenyl)acetic acid (397b) (38 mg, 36% yield) HCl salt as a white solid; ¹H NMR (300 MHz, DMSO-d₆) δ 13.23 (s, 1H, D₂O exchangeable), 12.08 (s, 1H, D₂O exchangeable), 9.11 (s, 2H, D₂O exchangeable), 8.89 (d, J=1.7 Hz, 1H), 8.32 (dd, J=8.5, 1.6 Hz, 1H), 8.04 (d, J=8.5 Hz, 1H), 7.94-7.84 (m, 2H), 7.69 (d, J=6.9 Hz, 1H), 7.57 (d, J=8.0 Hz, 1H), 7.36-7.19 (m, 4H), 6.98-6.88 (m, 1H), 5.95 (dd, J=6.8, 4.1 Hz, 1H), 4.12-3.93 (m, 2H), 3.63 (s, 3H), 3.47 (d, J=3.5 Hz, 2H), 3.15-2.82 (m, 2H), 2.74 (dd, J=13.8, 6.8 Hz, 1H), 2.08 (dd, J=13.7, 4.2 Hz, 1H), 1.98-1.74 (m, 2H), 1.71-1.47 (m, 2H); MS (ES+): 538.2 (M+1); (ES−): 536.2 (M−1); [α]_{D}=+89.6 [MeOH, 0.25].

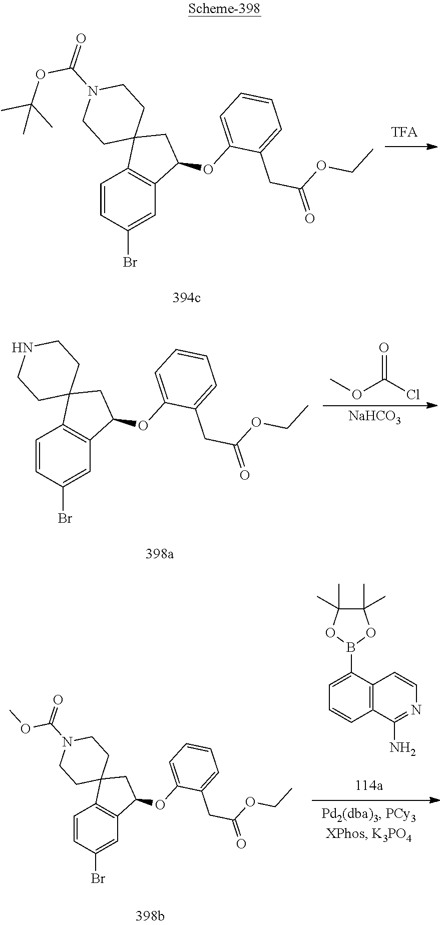

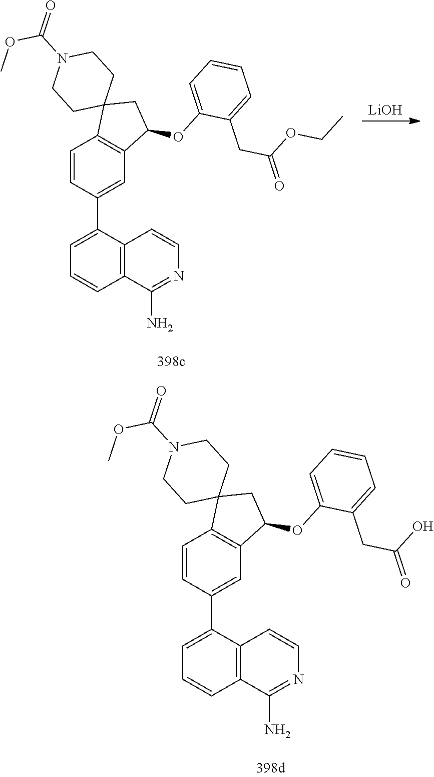

### Preparation of (R)-2-(2-((5-(1-aminoisoquinolin-5-yl)-1′-(methoxycarbonyl)-2,3-dihydrospiro[indene-1,4′-piperidin]-3-yl)oxy)phenyl)acetic acid (398d)

### Step-1: Preparation of (R)-ethyl 2-(2-((5-bromo-2,3-dihydrospiro[indene-1,4′-piperidin]-3-yl)oxy)phenyl)acetate (398a)

Compound 398a was prepared according to the procedure reported in step-4 of scheme-9, from (R)-tert-butyl 5-bromo-3-(2-(2-ethoxy-2-oxoethyl)phenoxy)-2,3-dihydrospiro[indene-1,4′-piperidine]-1′-carboxylate (394c) (450 mg, 0.826 mmol) in DCM (5 mL) using TFA (637 μl, 8.26 mmol) and stirring at RT for 2 h. The reaction mixture was concentrated to afford (R)-ethyl 2-(2-((5-bromo-2,3-dihydrospiro[indene-1,4′-piperidin]-3-yl)oxy)phenyl)acetate (398a) (360 mg, 98% yield) as clear oil which was used as such without further purification; MS (ES+): 444.1 and 446.1 (M+1); (ES−): 442.2 and 444.1 (M−1).

### Step-2: Preparation of (R)-methyl 5-bromo-3-(2-(2-ethoxy-2-oxoethyl)phenoxy)-2,3-dihydrospiro[indene-1,4′-piperidine]-1′-carboxylate (398b)

Compound 398b was prepared according to the procedure reported in step-1 of scheme-126, from (R)-ethyl 2-(2-((5-bromo-2,3-dihydrospiro[indene-1,4′-piperidin]-3-yl)oxy)phenyl)acetate, TFA (398a) (400 mg, 0.716 mmol) in THF (10 mL) using sodium bicarbonate (1791 μl, 3.58 mmol) and methyl carbonochloridate (83 μl, 1.075 mmol) to afford after workup and purification using method-P, (R)-methyl 5-bromo-3-(2-(2-ethoxy-2-oxoethyl)phenoxy)-2,3-dihydrospiro[indene-1,4′-piperidine]-1′-carboxylate (398b) (230 mg, 64% yield) as a clear oil; MS (ES+): 524.2 and 526.1 (M+Na).

### Step-3: Preparation of (R)-methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (398c)

Compound 398c was prepared according to the procedure reported in step-3 of scheme-112, from (R)-methyl 5-bromo-3-(2-(2-ethoxy-2-oxoethyl)phenoxy)-2,3-dihydrospiro[indene-1,4′-piperidine]-1′-carboxylate (398b) (100 mg, 0.199 mmol) in dioxane/2-MeTHF (2 mL) using 5-(4,4,5,5-tetramethyl-1,3,2-dioxaborolan-2-yl)isoquinolin-1-amine (114a) (108 mg, 0.398 mmol). K₃PO₄ (3M aqueous solution, 133 μl, 0.398 mmol), PCy₃ (22.33 mg, 0.080 mmol), XPhos (18.98 mg, 0.040 mmol), Pd₂(dba)₃ (36.5 mg, 0.040 mmol) and heating at 100° C. for 10 h to afford after workup and purification using method-U, (R)-methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (398c) (65 mg, 58% yield) as a clear oil; MS (ES+): 566.3 (M+1); [α]_{D}=−38.82 [MeOH, 0.17].

### Step-4: Preparation of (R)-2-(2-((5-(1-aminoisoquinolin-5-yl)-1′-(methoxycarbonyl)-2,3-dihydrospiro[indene-1,4′-piperidin]-3-yl)oxy)phenyl)acetic acid (398d)

Compound 398d was prepared according to the procedure reported in step-2 of scheme-1, from (R)-methyl 5-(1-aminoisoquinolin-5-yl)-3-(2-(2-ethoxy-2-oxoethyl)phenoxy)-2,3-dihydrospiro[indene-1,4′-piperidine]-1′-carboxylate (398c) (65 mg, 0.115 mmol) in THF (3 mL) using lithium hydroxide (14.30 mg, 0.597 mmol) in water (1 mL) to afford after work up and purification using method-M, (R)-2-(2-((5-(1-aminoisoquinolin-5-yl)-1′-(methoxycarbonyl)-2,3-dihydrospiro[indene-1,4′-piperidin]-3-yl)oxy)phenyl)acetic acid (398d) (44 mg, 41% yield) HCl salt as a white solid; ¹H NMR (300 MHz, DMSO-d₆) δ13.29 (s, 1H, D₂O exchangeable), 12.00 (s, 1H, D₂O exchangeable), 9.14 (s, 2H, D₂O exchangeable), 8.60 (d, J=8.2 Hz, 1H), 7.96-7.88 (m, 1H), 7.88-7.77 (m, 1H), 7.61 (d, J=7.2 Hz, 1H), 7.55 (d, J=7.8 Hz, 1H), 7.48-7.37 (m, 2H), 7.32-7.23 (m, 2H), 7.22-7.15 (m, 1H), 6.96 (d, J=7.3 Hz, 1H), 6.93-6.87 (m, 1H), 6.00-5.89 (m, 1H), 4.20-3.93 (m, 2H), 3.63 (s, 3H), 3.44 (d, J=1.4 Hz, 2H), 3.19-2.87 (m, 2H), 2.80 (dd, J=13.7, 6.9 Hz, 1H), 2.10 (dd, J=13.7, 4.5 Hz, 1H), 2.00-1.73 (m, 2H), 1.73-1.53 (m, 2H); MS (ES+): 538.2 (M+1); (ES−): 536.3 (M−1); [α]_{D}=−34.62 [MeOH, 0.26].

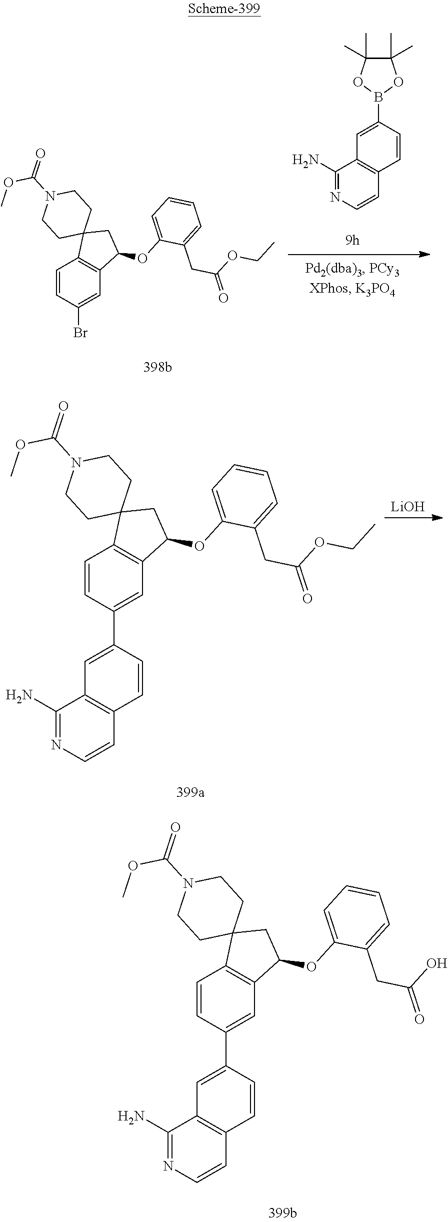

### Preparation of (R)-2-(2-((5-(1-aminoisoquinolin-7-yl)-1′-(methoxycarbonyl)-2,3-dihydrospiro[indene-1,4′-piperidin]-3-yl)oxy)phenyl)acetic acid (399b)

### Step-1: Preparation of (R)-methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (399a)

Compound 399a was prepared according to the procedure reported in step-3 of scheme-112, from (R)-methyl 5-bromo-3-(2-(2-ethoxy-2-oxoethyl)phenoxy)-2,3-dihydrospiro[indene-1,4′-piperidine]-1′-carboxylate (398b) (100 mg, 0.199 mmol) in dioxane/2-MeTHF (2 mL) using 7-(4,4,5,5-tetramethyl-1,3,2-dioxaborolan-2-yl)isoquinolin-1-amine (9h) (108 mg, 0.398 mmol), K₃PO₄ (3M aqueous solution, 133 μl, 0.398 mmol), PCy₃ (22.33 mg, 0.080 mmol), XPhos (18.98 mg, 0.040 mmol), Pd₂(dba)₃ (36.5 mg, 0.04 mmol) and heating at 100° C. for 10 h to afford after workup and purification using method-U, (R)-methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (399a) (63 mg, 56% yield) as a clear oil; MS (ES+): 566.3 (M+1); [α]_{D}=−93.6 [MeOH, 0.25].

### Step-2: Preparation of (R)-2-(2-((5-(1-aminoisoquinolin-7-yl)-1′-(methoxycarbonyl)-2,3-dihydrospiro[indene-1,4′-piperidin]-3-yl)oxy)phenyl)acetic acid (399b)

Compound 399b was prepared according to the procedure reported in step-2 of scheme-1, from (R)-methyl 5-(1-aminoisoquinolin-7-yl)-3-(2-(2-ethoxy-2-oxoethyl)phenoxy)-2,3-dihydrospiro[indene-1,4′-piperidine]-1′-carboxylate (399a) (63 mg, 0.111 mmol) in THF (3 mL) using lithium hydroxide (14.30 mg, 0.597 mmol) in water (I mL) to afford after work up and purification using method-M, (R)-2-(2-((5-(1-aminoisoquinolin-7-yl)-1′-(methoxycarbonyl)-2,3-dihydrospiro[indene-1,4′-piperidin]-3-yl)oxy)phenyl)acetic acid (399b) (33 mg, 31% yield) HCl salt as a white solid; JH NMR (300 MHz, DMSO-d₆) δ 13.28 (s, 1H, D₂O exchangeable), 12.08 (s, 1H, D₂O exchangeable), 9.13 (s, 2H, D₂O exchangeable), 8.89 (s, 1H), 8.31 (dd, J=8.6, 1.6 Hz, 1H), 8.03 (d, J=8.4 Hz, 1H), 7.95-7.83 (m, 2H), 7.69 (d, J=6.9 Hz, 1H), 7.57 (d, J=7.9 Hz, 1H), 7.37-7.16 (m, 4H), 7.02-6.85 (m, 1H), 6.04-5.80 (m, 1H), 4.12-3.93 (m, 2H), 3.63 (s, 3H), 3.47 (d, J=3.5 Hz, 2H), 3.15-2.82 (m, 2H), 2.80-2.66 (m, 1H), 2.08 (dd, J=13.6, 4.3 Hz, 1H), 1.95-1.71 (m, 2H), 1.69-1.46 (m, 2H); MS (ES+): 538.2 (M+1); (ES−): 536.2 (M−1); [α]_{D}=−106.4 [MeOH, 0.25].

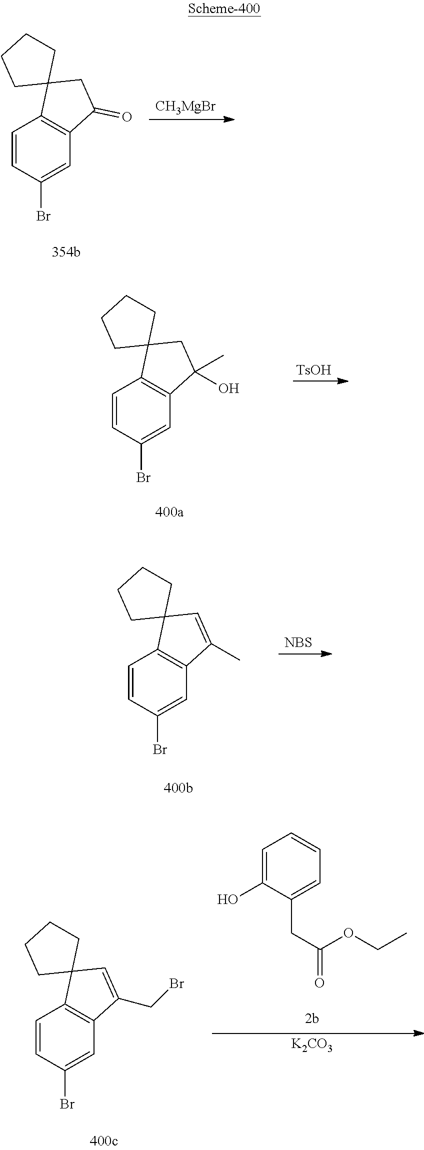

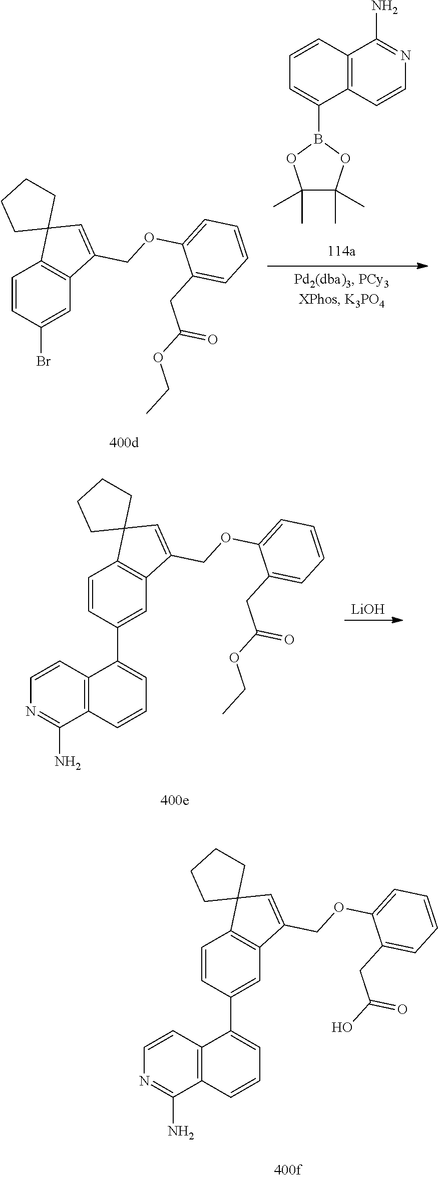

### Preparation of 2-(2-((5′-(1-aminoisoquinolin-5-yl)spiro[cyclopentane-1,1′-inden]-3′-yl)methoxy)phenyl)acetic acid (400f)

### Step-1: Preparation of 5′-bromo-3′-methyl-2′,3′-dihydrospiro[cyclopentane-1,1′-inden]-3′-ol (400a)

To a solution of methylmagnesium bromide (1M in THF; 15.09 mL, 15.09 mmol) was added 5′-bromospiro[cyclopentane-1,1′-inden]-3′(2′H)-one (354b) (2 g, 7.54 mmol) in THF (10 ml) at RT and the mixture was stirred overnight. The reaction mixture was cooled in an ice water bath, quenched carefully with NH₄Cl (sat.) (30 mL) and extracted with EtOAc (3×). Combined organics were washed with brine, dried, filtered and concentrated in vacuo. The residue obtained was purified using method-J to afford 5′-bromo-3′-methyl-2′,3′-dihydrospiro[cyclopentane-1,1′-inden]-3′-ol (400a) (1.8 g, 85% yield) as a clear oil; ¹H NMR (300 MHz, DMSO-d₆) δ 7.47-7.39 (m, 2H), 7.19 (d, J=8.7 Hz, 1H), 5.09 (s, 1H, D₂O exchangeable), 2.14-1.98 (m, 2H), 1.89-1.77 (m, 4H), 1.77-1.65 (m, 4H), 1.47 (s, 3H)

### Step-2: Preparation of 5′-bromo-3′-methylspiro[cyclopentane-1,1′-indene] (400b)

To a solution of 5′-bromo-3′-methyl-2′,3′-dihydrospiro[cyclopentane-1,1′-inden]-3′-ol (400a) (1.8 g, 6.40 mmol) in DCM (10 mL) was added TsOH (0.061 g, 0.320 mmol) at RT and stirred overnight at room temperature. The reaction mixture was washed with saturated NaHCO₃ solution, brine, dried, filtered and concentrated in vacuo. The residue obtained was purified using method-J to afford 5′-bromo-3′-methylspiro[cyclopentane-1,1′-indene] (400b) (1.35 g, 80% yield) as a clear oil; ¹H NMR (300 MHz, DMSO-d₆) δ 7.42-7.36 (m, 1H), 7.36-7.27 (m, 2H), 6.32-6.25 (m, 1H), 2.04 (d, J=1.6 Hz, 3H), 2.00-1.77 (m, 6H), 1.75-1.64 (m, 2H).

### Step-3: Preparation of 5′-bromo-3′-(bromomethyl)spiro[cyclopentane-1,1′-indene] (400c)

Compound 400c was prepared according to the procedure reported in step-2 of scheme-231, from 5′-bromo-3′-methylspiro[cyclopentane-1,1′-indene] (400b) (500 mg, 1.900 mmol) in carbon tetrachloride (10 mL) using NBS (338 mg, 1.900 mmol) and benzoyl peroxide (46.0 mg, 0.190 mmol) to afford after work up and purification using method-J, 5′-bromo-3′-(bromomethyl)spiro[cyclopentane-1,1′-indene] (400c) (200 mg, 31% yield); ¹H NMR (300 MHz, DMSO-d₆) δ 7.58-7.54 (m, 1H), 7.46-7.34 (m, 2H), 6.76 (s, 1H), 4.66 (s, 2H), 2.01-1.84 (m, 6H), 1.78-1.66 (m, 2H).

### Step-4: Preparation of ethyl 2-(2-((5′-bromospiro[cyclopentane-1,1′-inden]-3′-yl)methoxy)phenyl)acetate (400d)

Compound 400d was prepared according to the procedure reported in step-3 of scheme-106, from 5′-bromo-3′-(bromomethyl)spiro[cyclopentane-1,1′-indene] (400c) (200 mg, 0.585 mmol) in DMF (2 mL) using ethyl 2-(2-hydroxyphenyl)acetate (2b) (105 mg, 0.585 mmol), K₂CO₃ (162 mg, 1.169 mmol) and stirring at 60° C. for 1 h to afford after work up and purification using method-J, ethyl 2-(2-((5′-bromospiro[cyclopentane-1,1′-inden]-3′-yl)methoxy)phenyl)acetate (400d) (90 mg, 35% yield) as a white solid; ¹H NMR (300 MHZ, DMSO-d₆) δ 7.52 (d, J=1.2 Hz, 1H), 7.38 (d, J=1.1 Hz, 2H), 7.32-7.27 (m, 1H), 7.27-7.19 (m, 1H), 7.18-7.13 (m, 1H), 6.96-6.88 (m, 1H), 6.65-6.60 (m, 1H), 5.02 (d, J=1.5 Hz, 2H), 3.99 (q, J=7.1 Hz, 2H), 3.61 (s, 2H), 2.07-1.81 (m, 6H), 1.81-1.68 (m, 2H), 1.05 (t, J=7.1 Hz, 3H).

### Step-5: Preparation of ethyl 2-(2-((5′-(1-aminoisoquinolin-5-yl)spiro[cyclopentane-1,1′-inden]-3′-yl)methoxy)phenyl)acetate (400e)

Compound 400e was prepared according to the procedure reported in step-3 of scheme-112, from ethyl 2-(2-((5′-bromospiro[cyclopentane-1,1′-inden]-3′-yl)methoxy)phenyl)acetate (400d) (90 mg, 0.204 mmol) in dioxane/2-MeTHF (2 mL) using 5-(4,4,5,5-tetramethyl-1,3,2-dioxaborolan-2-yl)isoquinolin-1-amine (114a) (110 mg, 0.408 mmol), K₃PO₄ (3M aqueous solution, 136 μl, 0.408 mmol), PCy₃ (22.87 mg, 0.082 mmol), XPhos (19.44 mg, 0.041 mmol), Pd₂(dba)₃ (37.3 mg, 0.041 mmol) and heating at 100° C. for 10 h to afford after workup and purification using method-U, ethyl 2-(2-((5′-(1-aminoisoquinolin-5-yl)spiro[cyclopentane-1,1′-inden]-3′-yl)methoxy)phenyl)acetate (400e) (61 mg, 59% yield) as a clear oil; MS (ES+): 505.3 (M+1).

### Step-6: Preparation of 2-(2-((5′-(1-aminoisoquinolin-5-yl)spiro[cyclopentane-1,1′-inden]-3′-yl)methoxy)phenyl)acetic acid (400f)

Compound 400f was prepared according to the procedure reported in step-2 of scheme-1, from ethyl 2-(2-((5′-(1-aminoisoquinolin-5-yl)spiro[cyclopentane-1,1′-inden]-3′-yl)methoxy)phenyl)acetate (400e) (61 mg, 0.121 mmol) in THF (3 mL) using lithium hydroxide (14.65 mg, 0.612 mmol) in water (I mL) to afford after work up and purification using method-M, 2-(2-((5′-(1-aminoisoquinolin-5-yl)spiro[cyclopentane-1,1′-inden]-3′-yl)methoxy)phenyl)acetic acid (400f) (38 mg, 39% yield) HCl salt as a white solid; ¹H NMR (300 MHz, DMSO-d₆) δ 13.16 (s, 1H, D₂O exchangeable), 12.10 (s, 1H, D₂O exchangeable), 9.09 (s, 2H, DO exchangeable), 8.60 (d, J=8.3 Hz, 1H), 7.98-7.76 (m, 2H), 7.60 (t, J=9.2 Hz, 2H), 7.44 (s, 1H), 7.31-6.98 (m, 5H), 6.96-6.83 (m, 1H), 6.78-6.65 (m, 1H), 5.07 (s, 2H), 3.54 (s, 2H), 2.14-1.89 (m, 6H), 1.87-1.72 (m, 2H); MS (ES+): 477.2 (M+1); (ES−): 475.3 (M−1).

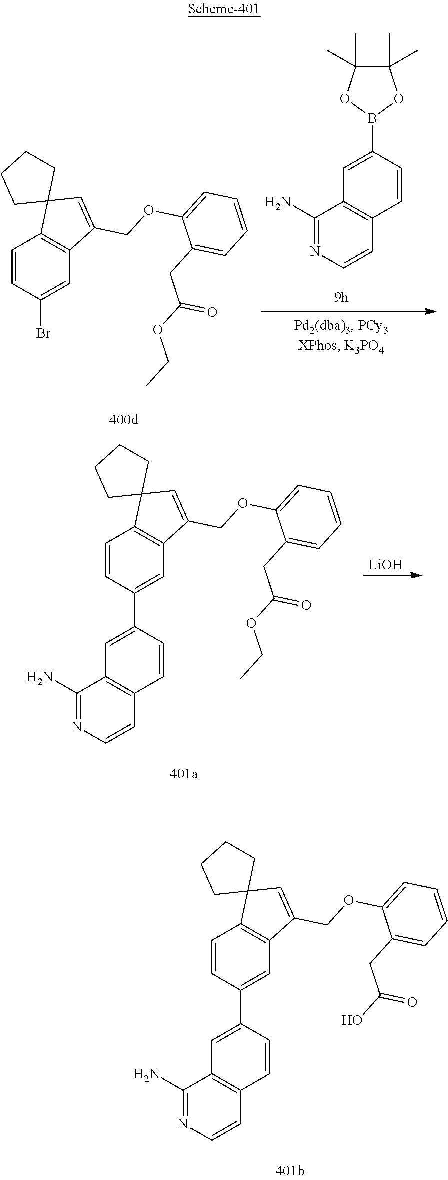

### Preparation of 2-(2-((5′-(1-aminoisoquinolin-7-yl)spiro[cyclopentane-1,1′-inden]-3′-yl)methoxy)phenyl)acetic acid (401b)

### Step-1: Preparation of ethyl 2-(2-((5′-(1-aminoisoquinolin-7-yl)spiro[cyclopentane-1,1′-inden]-3′-yl)methoxy)phenyl)acetate (401a)

Compound 401a was prepared according to the procedure reported in step-3 of scheme-112, from ethyl 2-(2-((5′-bromospiro[cyclopentane-1,1′-inden]-3′-yl)methoxy)phenyl)acetate (400d) (110 mg, 0.249 mmol) in dioxane/2-MeTHF (2 mL) using 7-(4,4,5,5-tetramethyl-1,3,2-dioxaborolan-2-yl)isoquinolin-1-amine (9h) (135 mg, 0.498 mmol), K₃PO₄ (3M aqueous solution, 166 μl, 0.498 mmol), PCy₃ (28.0 mg, 0.10 mmol), XPhos (23.76 mg, 0.050 mmol), Pd₂(dba)₃ (45.6 mg, 0.050 mmol) and heating at 100° C. for 10 h to afford after workup and purification using method-U, ethyl 2-(2-((5′-(1-aminoisoquinolin-7-yl)spiro[cyclopentane-1,1′-inden]-3′-yl)methoxy)phenyl)acetate (401a) (52 mg, 41% yield) as a clear oil; MS (ES+): 505.2 (M+1).

### Step-2: Preparation of 2-(2-((5′-(1-aminoisoquinolin-7-yl)spiro[cyclopentane-1,1′-inden]-3′-yl)methoxy)phenyl)acetic acid (401b)

Compound 400f was prepared according to the procedure reported in step-2 of scheme-1, from ethyl 2-(2-((5′-(1-aminoisoquinolin-7-yl)spiro[cyclopentane-1,1′-inden]-3′-yl)methoxy)phenyl)acetate (401a) (52 mg, 0.103 mmol) in THF (3 mL) using lithium hydroxide (17.90 mg, 0.748 mmol) in water (I mL) to afford after work up and purification using method-M, 2-(2-((5′-(1-aminoisoquinolin-7-yl)spiro[cyclopentane-1,1′-inden]-3′-yl)methoxy)phenyl)acetic acid (401b) (28 mg, 24% yield) HCl salt as a white solid; ¹H NMR (300 MHz, DMSO-d₆) & 13.14 (s, 1H, DO exchangeable), 12.17 (s, 1H, DO exchangeable), 9.12 (s, 2H, D₂O exchangeable), 8.94 (s, 1H), 8.36 (dd, J=8.5, 1.6 Hz, 1H), 8.05 (d, J=8.5 Hz, 1H), 7.87 (d, J=1.7 Hz, 1H), 7.75 (dd, J=7.9, 1.7 Hz, 1H), 7.69 (d, J=6.9 Hz, 1H), 7.59 (d, J=7.8 Hz, 1H), 7.32-7.14 (m, 4H), 6.92 (td, J=7.3, 1.2 Hz, 1H), 6.73-6.68 (m, 1H), 5.17 (s, 2H), 3.59 (s, 2H), 2.07-1.91 (m, 6H), 1.86-1.73 (m, 2H); MS (ES+): 477.2 (M+1); (ES−): 475.2 (M−1).

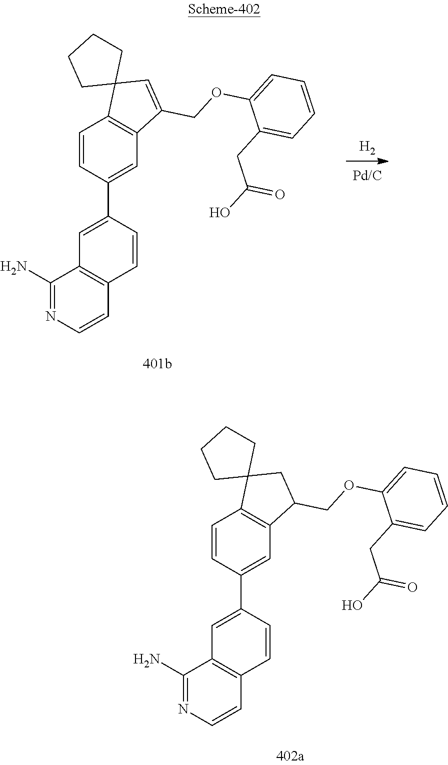 [SMILES: Nc1nccc2ccc(-c3ccc4c(c3)C(COc3ccccc3CC(=O)O)=CC43CCCC3)cc12 ; "H2" ; Nc1nccc2ccc(-c3ccc4c(c3)C(COc3ccccc3CC(=O)O)CC43CCCC3)cc12]

### Preparation of 2-(2-((5′-(1-aminoisoquinolin-7-yl)-2′,3′-dihydrospiro[cyclopentane-1,1′-inden]-3′-yl)methoxy)phenyl)acetic acid (402a)

Compound 402a was prepared according to the procedure reported in step-1 of scheme-302, from 2-(2-((5′-(1-aminoisoquinolin-7-yl)spiro[cyclopentane-1,1′-inden]-3′-yl)methoxy)phenyl)acetic acid (401b) (22 mg, 0.046 mmol) in MeOH (10 mL) using Pd/C (10% wt, 9.83 mg) and stirring for 2 h at RT under a H₂ atmosphere to afford after work up and purification using method-G, 2-(2-((5′-(1-aminoisoquinolin-7-yl)-2′,3′-dihydrospiro[cyclopentane-1,1′-inden]-3′-yl)methoxy)phenyl)acetic acid (402a) (20 mg, 91% yield) HCl salt as a white solid; ¹H NMR (300 MHz, DMSO-d₆) δ 13.27 (s, 1H, D₂O exchangeable), 12.10 (s, 1H, D₂O exchangeable), 9.17 (s, 2H, DO exchangeable), 8.96-8.88 (m, 1H), 8.31 (dd, J=8.5, 1.6 Hz, 1H), 8.02 (d, J=8.5 Hz, 1H), 7.89 (s, 1H), 7.78 (dd, J=8.0, 1.8 Hz, 1H), 7.68 (d, J=6.8 Hz, 1H), 7.40 (d, J=7.9 Hz, 1H), 7.29-7.15 (m, 3H), 7.10 (d, J=8.1 Hz, 1H), 6.89 (td, J=7.4, 1.1 Hz, 1H), 4.57-4.45 (m, 1H), 4.03 (t, J=8.7 Hz, 1H), 3.71-3.61 (m, 1H), 3.54 (s, 2H), 2.41-2.27 (m, 1H), 2.09-1.96 (m, 1H), 1.90-1.71 (m, 8H); MS (ES+): 479.3 (M+1); (ES−): 477.3 (M−1).

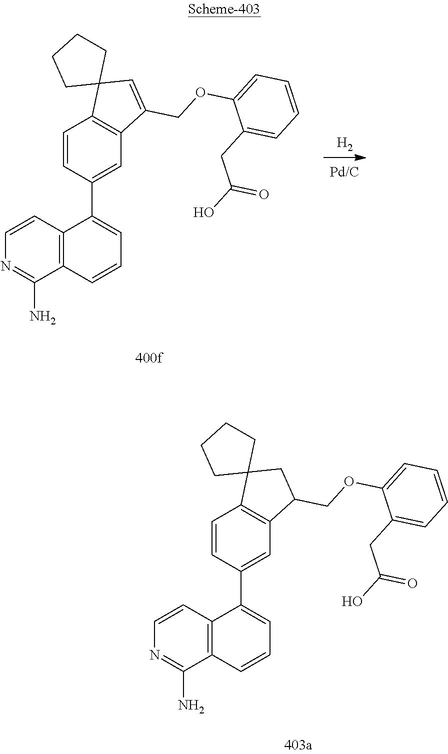 [SMILES: Nc1nccc2c(-c3ccc4c(c3)C(COc3ccccc3CC(=O)O)=CC43CCCC3)cccc12 ; "H2" ; Nc1nccc2c(-c3ccc4c(c3)C(COc3ccccc3CC(=O)O)CC43CCCC3)cccc12]

### Preparation of 2-(2-((5′-(1-aminoisoquinolin-5-yl)-2′,3′-dihydrospiro[cyclopentane-1,1′-inden]-3′-yl)methoxy)phenyl)acetic acid (403a)

Compound 403a was prepared according to the procedure reported in step-1 of scheme-302, from 2-(2-((5′-(1-aminoisoquinolin-5-yl)spiro[cyclopentane-1,1′-inden]-3′-yl)methoxy)phenyl)acetic acid (400f) (23 mg, 0.048 mmol) in MeOH (10 mL) using Pd/C (10% wt, 10.27 mg) and stirring for 2 h at RT under a H₂ atmosphere to afford after work up and purification using method-G, 2-(2-((5′-(1-aminoisoquinolin-5-yl)-2′,3′-dihydrospiro[cyclopentane-1, I′-inden]-3′-yl)methoxy)phenyl)acetic acid (403a) (5 mg, 22% yield) HCl salt as a white solid; ¹H NMR (300 MHz, DMSO-d₆) δ 13.11 (s, 1H, D₂O exchangeable), 12.04 (s, 1H, D₂O exchangeable), 9.04 (s, 2H, D₂O) exchangeable), 8.57 (d, J=8.1 Hz, 1H), 7.93-7.86 (m, 1H), 7.86-7.77 (m, 1H), 7.56 (d, J=7.2 Hz, 1H), 7.45 (s, 1H), 7.40 (d, J=7.8 Hz, 1H), 7.29 (dd, J=7.8, 1.6 Hz, 1H), 7.25-7.12 (m, 2H), 7.07-6.95 (m, 2H), 6.87 (td, J=7.4, 1.1 Hz, 1H), 4.43-4.32 (m, 1H), 4.12-3.99 (m, 1H), 3.72-3.59 (m, 1H), 3.44 (d, J=3.4 Hz, 2H), 2.39-2.29 (m, 1H), 2.10-1.97 (m, 1H), 1.92-1.57 (m, 8H); MS (ES+): 479.2 (M+1); (ES−): 477.2 (M−1).

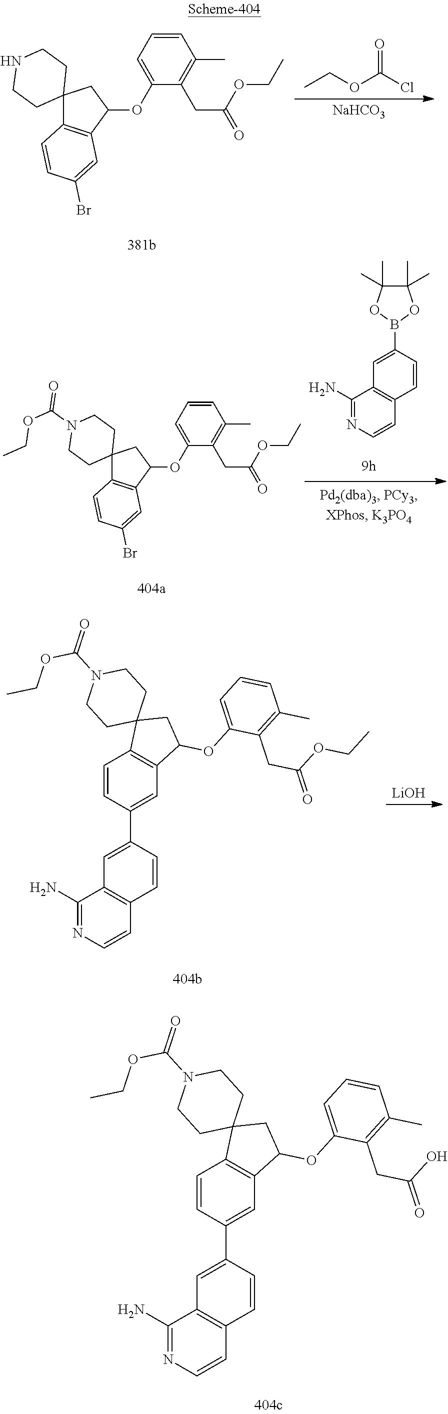

### Preparation of 2-(2-((5-(1-aminoisoquinolin-7-yl)-1′-(ethoxycarbonyl)-2,3-dihydrospiro[indene-1,4′-piperidin]-3-yl)oxy)-6-methylphenyl)acetic acid (404c)

### Step-1: Preparation of ethyl 5-bromo-3-(2-(2-ethoxy-2-oxoethyl)-3-methylphenoxy)-2,3-dihydrospiro[indene-1,4′-piperidine]-1′-carboxylate (404a)

Compound 404a was prepared according to the procedure reported in step-1 of scheme-370, from ethyl 2-(2-((5-bromo-2,3-dihydrospiro[indene-1,4′-piperidin]-3-yl)oxy)-6-methylphenyl)acetate, TFA (381b) (0.572 g, 1 mmol) in THF (10 mL) using sodium bicarbonate (2.500 mL, 5.00 mmol) and ethyl carbonochloridate (0.143 mL, 1.50 mmol) to afford after workup and purification using method-P, ethyl 5-bromo-3-(2-(2-ethoxy-2-oxoethyl)-3-methylphenoxy)-2,3-dihydrospiro[indene-1,4′-piperidine]-1′-carboxylate (404a) (0.32 g, 60% yield) as a clear oil; MS (ES+): 552.1 (M+Na).

### Step-2: Preparation of ethyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (404b)

Compound 404b was prepared according to the procedure reported in step-3 of scheme-112, from ethyl 5-bromo-3-(2-(2-ethoxy-2-oxoethyl)-3-methylphenoxy)-2,3-dihydrospiro[indene-1,4′-piperidine]-1′-carboxylate (404a) (160 mg, 0.302 mmol) in dioxane/2-MeTHF (2 mL) using 7-(4,4,5,5-tetramethyl-1,3,2-dioxaborolan-2-yl)isoquinolin-1-amine (9h) (163 mg, 0.603 mmol), K₃PO₄ (3M aqueous solution, 201 μl, 0.603 mmol), PCy₃ (33.8 mg, 0.121 mmol), XPhos (14.38 mg, 0.03 mmol). Pd₂(dba)₃ (55.2 mg, 0.06 mmol) and heating at 100° C. for 10 h to afford after workup and purification using method-F followed by method-M, ethyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (404b) (42 mg, 23% yield) as a white solid; MS (ES+): 594.3 (M+1).

### Step-3: Preparation of 2-(2-((5-(1-aminoisoquinolin-7-yl)-1′-(ethoxycarbonyl)-2,3-dihydrospiro[indene-1,4′-piperidin]-3-yl)oxy)-6-methylphenyl)acetic acid (404c)

Compound 404c was prepared according to the procedure reported in step-2 of scheme-1, from ethyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (404b) (40 mg, 0.067 mmol) in MeOH (1 mL) and THF (1 mL) using lithium hydroxide (1 N solution, 202 μl, 0.202 mmol) to afford after work up and purification using method-G, 2-(2-((5-(1-aminoisoquinolin-7-yl)-1′-(ethoxycarbonyl)-2,3-dihydrospiro[indene-1,4′-piperidin]-3-yl)oxy)-6-methylphenyl)acetic acid (404c) (22 mg, 58% yield) HCl salt as a white solid; ¹H NMR (300 MHz, DMSO-d₆) δ 13.23 (s, 1H, D₂O exchangeable), 12.12 (s, 1H, D₂O) exchangeable), 9.11 (s, 2H, D₂O exchangeable), 8.89 (s, 1H), 8.31 (dd, J=8.5, 1.6 Hz, 1H), 8.04 (d, J=8.5 Hz, 1H), 7.95-7.82 (m, 2H), 7.69 (d, J=6.9 Hz, 1H), 7.57 (d, J=8.0 Hz, 1H), 7.32-7.10 (m, 3H), 6.85-6.76 (m, 1H), 6.05-5.83 (m, 1H), 4.16-3.89 (m, 4H), 3.62-3.41 (m, 2H), 3.14-2.77 (m, 2H), 2.70 (dd, J=13.8, 6.8 Hz, 1H), 2.22 (s, 3H), 2.16-1.99 (m, 1H), 1.97-1.72 (m, 2H), 1.69-1.44 (m, 2H), 1.21 (t, J=7.1 Hz, 3H); MS (ES+): 566.3 (M+1); (ES−): 564.3 (M−1).

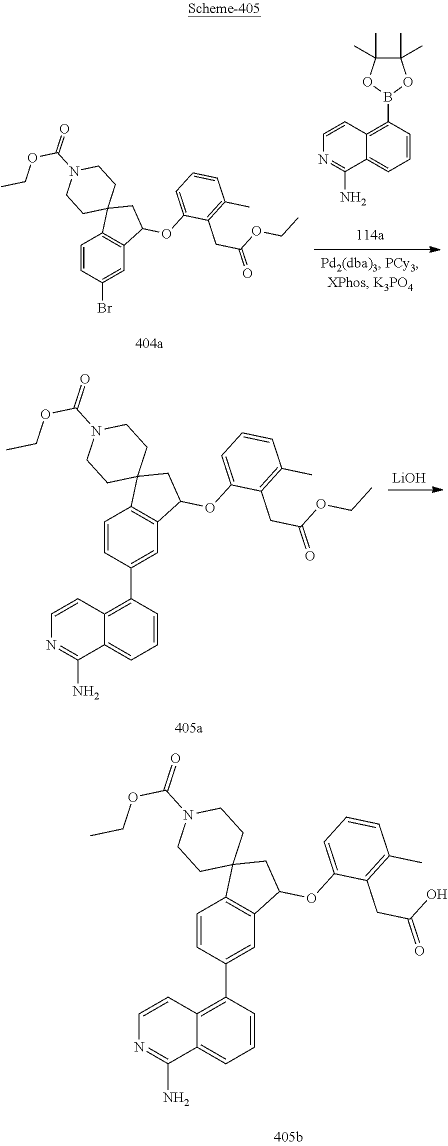

### Preparation of 2-(2-((5-(1-aminoisoquinolin-5-yl)-1′-(ethoxycarbonyl)-2,3-dihydrospiro[indene-1,4′-piperidin]-3-yl)oxy)-6-methylphenyl)acetic acid (405b)

### Step-1: Preparation of ethyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (405a)

Compound 405a was prepared according to the procedure reported in step-3 of scheme-112, from ethyl 5-bromo-3-(2-(2-ethoxy-2-oxoethyl)-3-methylphenoxy)-2,3-dihydrospiro[indene-1,4′-piperidine]-1′-carboxylate (404a) (160 mg, 0.302 mmol) in dioxane/2-MeTHF (2 mL) using 5-(4,4,5,5-tetramethyl-1,3,2-dioxaborolan-2-yl)isoquinolin-1-amine (114a) (163 mg, 0.603 mmol), K₃PO₄ (3M aqueous solution, 201 μl, 0.603 mmol), PCy₃ (33.8 mg, 0.121 mmol), XPhos (14.38 mg, 0.030 mmol), Pd₂(dba)₃ (55.2 mg, 0.06 mmol) and heating at 100° C. for 10 h to afford after workup and purification using method-M, ethyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (405a) (65 mg, 36% yield) as a pale yellow solid; MS (ES+): 594.3 (M+1).

### Step-2: Preparation of 2-(2-((5-(1-aminoisoquinolin-5-yl)-1′-(ethoxycarbonyl)-2,3-dihydrospiro[indene-1,4′-piperidin]-3-yl)oxy)-6-methylphenyl)acetic acid (405b)

Compound 405b was prepared according to the procedure reported in step-2 of scheme-1, from ethyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (405a) (50 mg, 0.084 mmol) in MeOH (1 mL) and THF (1 mL) using lithium hydroxide (1 N solution, 253 μl, 0.253 mmol) to afford after work up and purification using method-G, 2-(2-((5-(1-aminoisoquinolin-5-yl)-1′-(ethoxycarbonyl)-2,3-dihydrospiro[indene-1,4′-piperidin]-3-yl)oxy)-6-methylphenyl)acetic acid (405b) (32 mg, 67% yield) HCl salt as a white solid; ¹H NMR (300 MHz, DMSO-d₆) δ 13.26 (s, 1H, D₂O exchangeable), 12.06 (s, 1H, D₂O exchangeable), 9.11 (s, 2H, D₂O) exchangeable), 8.60 (d, J=8.2 Hz, 1H), 7.94-7.75 (m, 2H), 7.61 (d, J=7.2 Hz, 1H), 7.56 (d, J=7.9 Hz, 1H), 7.47-7.35 (m, 2H), 7.21-7.04 (m, 2H), 6.96 (d, J=7.3 Hz, 1H), 6.80 (dd, J=6.8, 2.0 Hz, 1H), 5.97-5.85 (m, 1H), 4.19-3.86 (m, 4H), 3.50 (s, 2H), 3.20-2.82 (m, 2H), 2.76 (dd, J=13.8, 6.9 Hz, 1H), 2.20 (s, 3H), 2.10 (dd, J=13.1, 3.9 Hz, 1H), 2.00-1.86 (m, 1H), 1.86-1.72 (m, 1H), 1.71-1.53 (m, 2H), 1.21 (t, J=7.1 Hz, 3H); MS (ES+): 566.3 (M+1); (ES−): 564.3 (M−1).

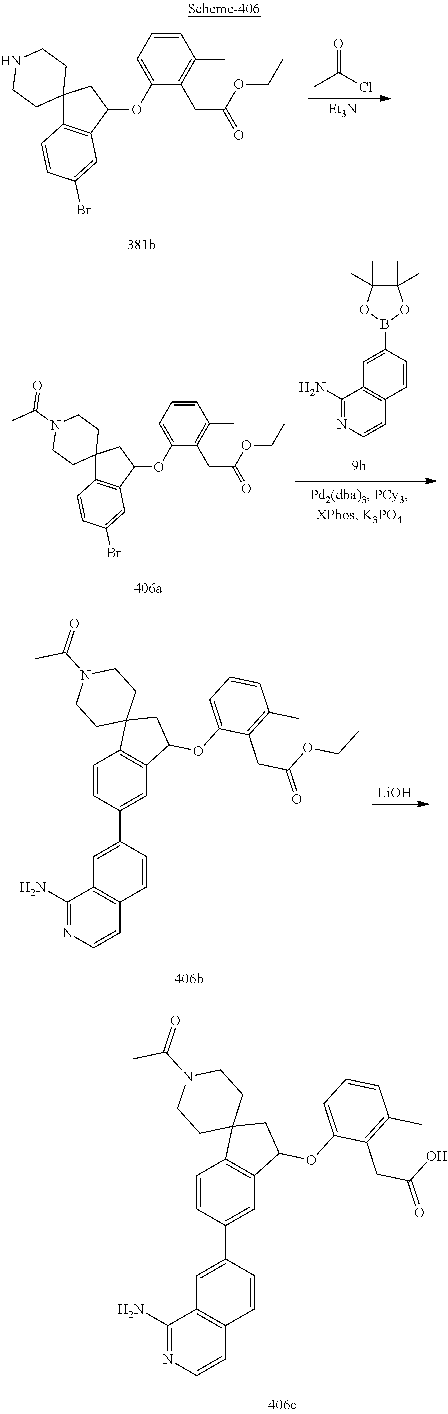

### Preparation of 2-(2-((1′-acetyl-5-(1-aminoisoquinolin-7-yl)-2,3-dihydrospiro[indene-1,4′-piperidin]-3-yl)oxy)-6-methylphenyl)acetic acid (406c)

### Step-1: Preparation of ethyl 2-(2-((1′-acetyl-5-bromo-2,3-dihydrospiro[indene-1,4′-piperidin]-3-yl)oxy)-6-methylphenyl)acetate (406a)

Compound 406a was prepared according to the procedure reported in step-1 of scheme-385, from ethyl 2-(2-((5-bromo-2,3-dihydrospiro[indene-1,4′-piperidin]-3-yl)oxy)-6-methylphenyl)acetate, TFA (381b) (0.7 g, 1.223 mmol) using triethylamine (0.545 mL, 3.91 mmol) and stirring for 2 h at RT to afford after work up and purification using method-O, ethyl 2-(2-((1′-acetyl-5-bromo-2,3-dihydrospiro[indene-1,4′-piperidin]-3-yl)oxy)-6-methylphenyl)acetate (406a) (0.49 g, 80% yield) as a clear gel; MS (ES+): 500.1 (M+1).

### Step-2: Preparation of ethyl 2-(2-((1′-acetyl-5-(1-aminoisoquinolin-7-yl)-2,3-dihydrospiro[indene-1,4′-piperidin]-3-yl)oxy)-6-methylphenyl)acetate (406b)

Compound 406b was prepared according to the procedure reported in step-3 of scheme-112, from ethyl 2-(2-((1′-acetyl-5-bromo-2,3-dihydrospiro[indene-1,4′-piperidin]-3-yl)oxy)-6-methylphenyl)acetate (406a) (240 mg, 0.48 mmol) in dioxane/2-MeTHF (3 mL) using 7-(4,4,5,5-tetramethyl-1,3,2-dioxaborolan-2-yl)isoquinolin-1-amine (9h) (259 mg, 0.959 mmol), K₃PO₄ (3M aqueous solution, 320 μl, 0.959 mmol), PCy₃ (53.8 mg, 0.192 mmol), XPhos (22.86 mg, 0.048 mmol), Pd₂(dba)₃ (88 mg, 0.096 mmol) and heating at 100° C. for 10 b to afford after workup and purification using method-F ethyl 2-(2-((1′-acetyl-5-(1-aminoisoquinolin-7-yl)-2,3-dihydrospiro[indene-1,4′-piperidin]-3-yl)oxy)-6-methylphenyl)acetate (406b) (51 mg, 19% yield) as a pale yellow solid; MS (ES+): 564.3 (M+1).

### Step-3: Preparation of 2-(2-((1′-acetyl-5-(1-aminoisoquinolin-7-yl)-2,3-dihydrospiro[indene-1,4′-piperidin]-3-yl)oxy)-6-methylphenyl)acetic acid (406c)

Compound 406c was prepared according to the procedure reported in step-2 of scheme-1, from ethyl 2-(2-((1′-acetyl-5-(1-aminoisoquinolin-7-yl)-2,3-dihydrospiro[indene-1,4′-piperidin]-3-yl)oxy)-6-methylphenyl)acetate (406b)(47 mg, 0.083 mmol) in MeOH (I mL) and THF (1 mL) using lithium hydroxide (1N solution, 334 μl, 0.334 mmol) to afford after work up and purification using method-G, 2-(2-((1′-acetyl-5-(1-aminoisoquinolin-7-yl)-2,3-dihydrospiro[indene-1,4′-piperidin]-3-yl)oxy)-6-methylphenyl)acetic acid (406c) (25 mg, 56% yield) HCl salt as a white solid; ¹H NMR (300 MHz, DMSO-d₆) δ 13.00 (s, 1H, D₂O exchangeable), 12.10 (s, 1H, D₂O exchangeable), 9.04 (s, 2H, D₂O exchangeable), 8.88 (s, 1H), 8.38-8.23 (m, 1H), 8.04 (d, J=8.5 Hz, 1H), 7.98-7.80 (m, 2H), 7.68 (d, J=6.9 Hz, 1H), 7.56 (d, J=8.0 Hz, 1H), 7.28 (d, J=7.0 Hz, 1H), 7.24-7.11 (m, 2H), 6.87-6.79 (m, 1H), 5.98-5.89 (m, 1H), 4.53-4.33 (m, 1H), 3.97-3.73 (m, 1H), 3.65-3.03 (m, 4H), 2.88-2.53 (m, 2H), 2.22 (s, 3H), 2.18-2.08 (m, 1H), 2.05 (d, J=4.3 Hz, 3H), 1.99-1.71 (m, 1H), 1.71-1.48 (m, 2H); MS (ES+): 536.2 (M+1); (ES−): 534.3 (M−1).

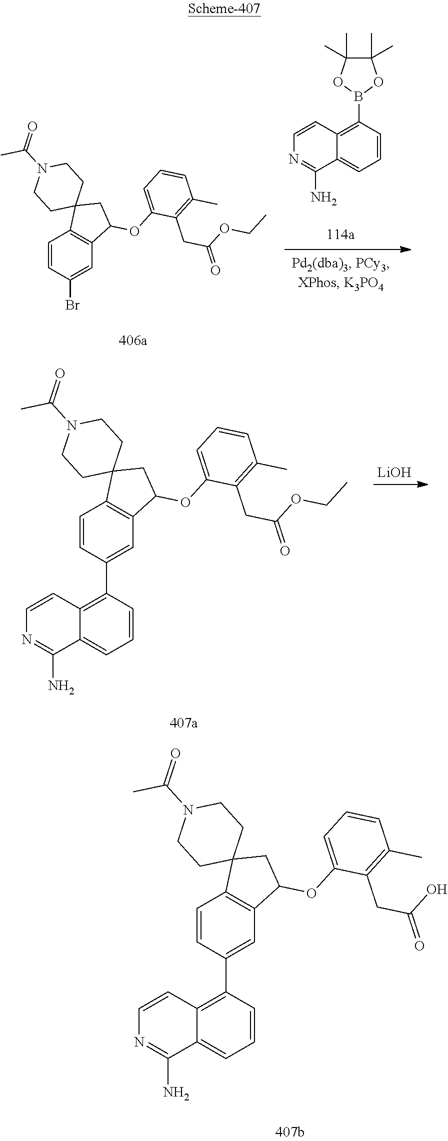

### Preparation of 2-(2-((1′-acetyl-5-(1-aminoisoquinolin-5-yl)-2,3-dihydrospiro[indene-1,4′-piperidin]-3-yl)oxy)-6-methylphenyl)acetic acid (407b)

### Step-1: Preparation of ethyl 2-(2-((1′-acetyl-5-(1-aminoisoquinolin-5-yl)-2,3-dihydrospiro[indene-1,4′-piperidin]-3-yl)oxy)-6-methylphenyl)acetate (407a)

Compound 407a was prepared according to the procedure reported in step-3 of scheme-112, from ethyl 2-(2-((1′-acetyl-5-bromo-2,3-dihydrospiro[indene-1,4′-piperidin]-3-yl)oxy)-6-methylphenyl)acetate (406a) (240 mg, 0.480 mmol) in dioxane/2-MeTHF (3 mL) using 5-(4,4,5,5-tetramethyl-1,3,2-dioxaborolan-2-yl)isoquinolin-1-amine (114a) (259 mg, 0.959 mmol), K₃PO₄ (3M aqueous solution, 320 μl, 0.959 mmol), PCy₃ (53.8 mg, 0.192 mmol), XPhos (22.86 mg, 0.048 mmol), Pd₂(dba)₃ (88 mg, 0.096 mmol) and heating at 100° C. for 10 b to afford after workup and purification using method-F, ethyl 2-(2-((1′-acetyl-5-(1-aminoisoquinolin-5-yl)-2,3-dihydrospiro[indene-1,4′-piperidin]-3-yl)oxy)-6-methylphenyl)acetate (407a) (145 mg, 54% yield) as a pale yellow solid; MS (ES+): 564.3 (M+1).

### Step-2: Preparation of 2-(2-((1′-acetyl-5-(1-aminoisoquinolin-5-yl)-2,3-dihydrospiro[indene-1,4′-piperidin]-3-yl)oxy)-6-methylphenyl)acetic acid (407b)

Compound 407b was prepared according to the procedure reported in step-2 of scheme-1, from ethyl 2-(2-((1′-acetyl-5-(1-aminoisoquinolin-5-yl)-2,3-dihydrospiro[indene-1,4′-piperidin]-3-yl)oxy)-6-methylphenyl)acetate (407a) (140 mg, 0.248 mmol) in MeOH (2 mL) and THF (2 mL) using lithium hydroxide (1 N solution, 993 μl, 0.993 mmol) to afford after work up and purification using method-G, 2-(2-((1′-acetyl-5-(1-aminoisoquinolin-5-yl)-2,3-dihydrospiro[indene-1,4′-piperidin]-3-yl)oxy)-6-methylphenyl)acetic acid (407b) (81 mg, 61% yield) HCl salt as a white solid; 3H NMR (300 MHz, DMSO-d₆) δ 13.37 (s, 1H, D₂O exchangeable), 12.01 (s, 1H, D₂O) exchangeable), 9.15 (s, 2H, D₂O exchangeable), 8.61 (d, J=8.2 Hz, 1H), 7.91 (dd, J=7.3, 1.2 Hz, 1H), 7.87-7.77 (m, 1H), 7.62 (d, J=7.2 Hz, 1H), 7.54 (d, J=7.8 Hz, 1H), 7.49-7.37 (m, 2H), 7.21-7.08 (m, 2H), 6.96 (d, J=7.2 Hz, 1H), 6.80 (dd, J=6.4, 2.2 Hz, 1H), 5.92 (dd, J=6.8, 4.4 Hz, 1H), 4.55-4.34 (m, 1H), 3.85 (t, J=13.5 Hz, 1H), 3.50 (s, 2H), 3.29-3.09 (m, 2H), 2.91-2.56 (m, 2H), 2.20 (s, 3H), 2.17-2.02 (m, 4H), 1.98-1.77 (m, 1H), 1.75-1.50 (m, 2H); MS (ES+): 536.3 (M+1); (ES−): 534.3 (M−1).

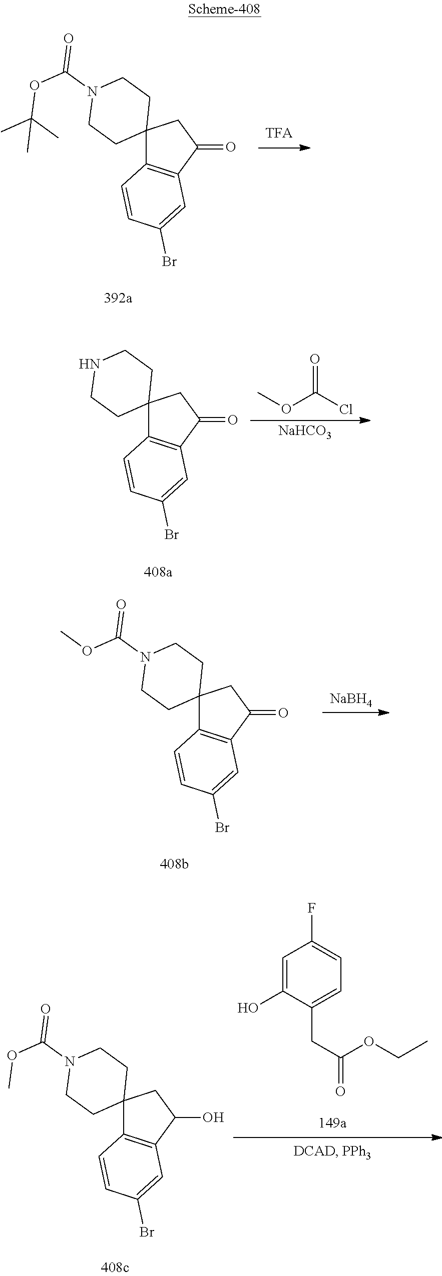

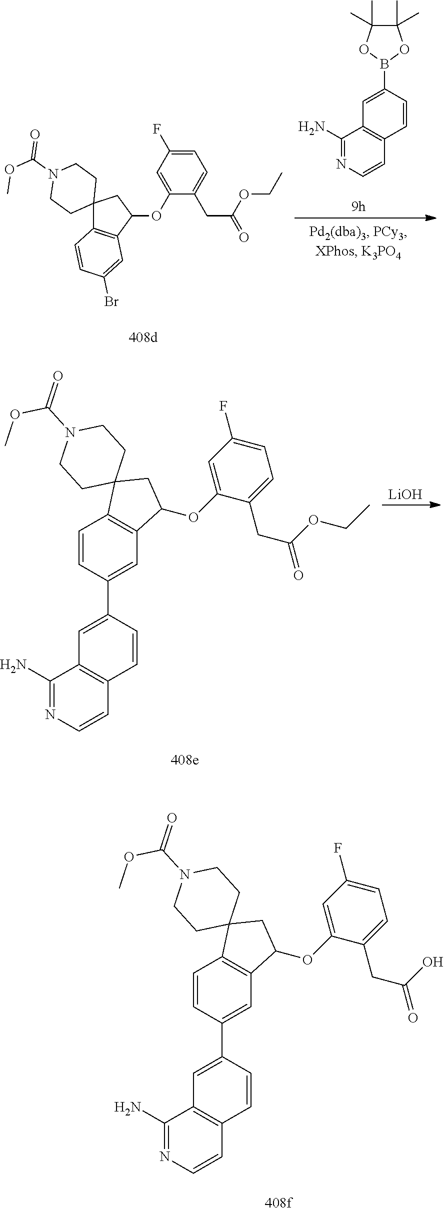

### Preparation of 2-(2-((5-(1-aminoisoquinolin-7-yl)-1′-(methoxycarbonyl)-2,3-dihydrospiro[indene-1,4′-piperidin]-3-yl)oxy)-4-fluorophenyl)acetic acid (408f)

### Step-1: Preparation of 5-bromospiro[indene-1,4′-piperidin]-3(2H)-one (408a)

Compound 408a was prepared according to the procedure reported in step-4 of scheme-9, from tert-butyl 5-bromo-3-oxo-2,3-dihydrospiro[indene-1,4′-piperidine]-1′-carboxylate (392a) (10 g, 26.3 mmol) in DCM (100 mL) using TFA (10.13 mL, 131 mmol) to afford after work up and purification using method-BU, 5-bromospiro[indene-1,4′-piperidin]-3(2H)-one (408a), 2TFA (13.36 g, >99% yield); MS (ES+): 280.1 (M+1).

### Step-2: Preparation of methyl 5-bromo-3-oxo-2,3-dihydrospiro[indene-1,4′-piperidine]-1′-carboxylate (408b)

Compound 408b was prepared according to the procedure reported in step-1 of scheme-126, from 5-bromospiro[indene-1,4′-piperidin]-3(2H)-one (408a), 2TFA (13.21 g, 26 mmol) in THF (200 mL) using sodium bicarbonate (91 mL, 182 mmol) and methyl carbonochloridate (3.62 mL, 46.8 mmol) to afford after workup and purification using method-I, methyl 5-bromo-3-oxo-2,3-dihydrospiro[indene-1,4′-piperidine]-1′-carboxylate (408b) (8.05 g, 92% yield) as a pale yellow solid; MS (ES+): 338.1 (M+1).

### Step-3: Preparation of methyl 5-bromo-3-hydroxy-2,3-dihydrospiro[indene-1,4′-piperidine]-1′-carboxylate (408c)

Compound 408c was prepared according to the procedure reported in step-1 of scheme-205, from methyl 5-bromo-3-oxo-2,3-dihydrospiro[indene-1,4′-piperidine]-1′-carboxylate (408b) (6 g, 17.74 mmol) in anhydrous MeOH (75 mL) using sodium borohydride (0.839 g, 22.18 mmol) to afford after work up and purification using method-I, methyl 5-bromo-3-hydroxy-2,3-dihydrospiro[indene-1,4′-piperidine]-1′-carboxylate (408c) (5.95 g, 99% yield) as a pale yellow oil; MS (ES+): 340.0 (M+1).

### Step-4: Preparation of methyl 5-bromo-3-(2-(2-ethoxy-2-oxoethyl)-5-fluorophenoxy)-2,3-dihydrospiro[indene-1,4′-piperidine]-1′-carboxylate (408d)

Compound 408d was prepared according to the procedure reported in step-2 of scheme-2, from methyl 5-bromo-3-hydroxy-2,3-dihydrospiro[indene-1,4′-piperidine]-1′-carboxylate (408c) (0.6 g, 1.764 mmol) in DCM (30 mL) using PPh₃ (0.555 g, 2.116 mmol), ethyl 2-(4-fluoro-2-hydroxyphenyl)acetate (149a) (0.419 g, 2.116 mmol) and a solution of DCAD (0.777 g, 2.116 mmol) in DCM (10 mL) to afford after workup and purification using method-O, methyl 5-bromo-3-(2-(2-ethoxy-2-oxoethyl)-5-fluorophenoxy)-2,3-dihydrospiro[indene-1,4′-piperidine]-1′-carboxylate (408d) (0.5 g, 0.961 mmol, 54.5% yield) as a clear gel; MS (ES+): 520.1 (M+1).

### Step-5: Preparation of methyl 5-(1-aminoisoquinolin-7-yl)-3-(2-(2-ethoxy-2-oxoethyl)-5-fluorophenoxy)-2,3-dihydrospiro[indene-1,4′-piperidine]-1′-carboxylate (408e)

Compound 408e was prepared according to the procedure reported in step-3 of scheme-112, from methyl 5-bromo-3-(2-(2-ethoxy-2-oxoethyl)-5-fluorophenoxy)-2,3-dihydrospiro[indene-1,4′-piperidine]-1′-carboxylate (408d) (250 mg, 0.480 mmol) in dioxane/2-MeTHF (3 mL) using 7-(4,4,5,5-tetramethyl-1,3,2-dioxaborolan-2-yl)isoquinolin-1-amine (9h) (260 mg, 0.961 mmol), K₃PO₄ (3M aqueous solution, 320 μl, 0.961 mmol), PCy₃ (53.9 mg, 0.192 mmol). XPhos (22.90 mg, 0.048 mmol) and Pd₂(dba)₃ (88 mg, 0.096 mmol) and heating at 100° C. for 7 h to afford after workup and purification using method-F, methyl 5-(1-aminoisoquinolin-7-yl)-3-(2-(2-ethoxy-2-oxoethyl)-5-fluorophenoxy)-2,3-dihydrospiro[indene-1,4′-piperidine]-1′-carboxylate (408e) (65 mg, 23% yield) as a pale yellow solid; MS (ES+): 584.2 (M+1).

### Step-6: Preparation of 2-(2-((5-(1-aminoisoquinolin-7-yl)-1′-(methoxycarbonyl)-2,3-dihydrospiro[indene-1,4′-piperidin]-3-yl)oxy)-4-fluorophenyl)acetic acid (4081)

Compound 408f was prepared according to the procedure reported in step-2 of scheme-1, from methyl 5-(1-aminoisoquinolin-7-yl)-3-(2-(2-ethoxy-2-oxoethyl)-5-fluorophenoxy)-2,3-dihydrospiro[indene-1,4′-piperidine]-1′-carboxylate (408e) (62 mg, 0.106 mmol) in MeOH (1 mL) and THF (1 mL) using lithium hydroxide (1 N solution, 531 μl, 0.531 mmol) to afford after work up and purification using method-G, 2-(2-((5-(1-aminoisoquinolin-7-yl)-1′-(methoxycarbonyl)-2,3-dihydrospiro[indene-1,4′-piperidin]-3-yl)oxy)-4-fluorophenyl)acetic acid (408f) (27 mg, 46% yield) HCl salt as a white solid; ¹H NMR (300 MHz, DMSO-d₆) δ 13.16 (s, 1H, D₂O exchangeable), 12.15 (s, 1H, D₂O exchangeable), 8.99 (s, 2H, D₂O exchangeable), 8.87 (s, 1H), 8.30 (dd, J=8.5, 1.6 Hz, 1H), 8.03 (d, J=8.5 Hz, 1H), 7.95-7.82 (m, 2H), 7.69 (d, J=6.9 Hz, 1H), 7.57 (d, J=8.0 Hz, 1H), 7.31-7.16 (m, 3H), 6.75 (td, J=8.5, 2.4 Hz, 1H), 5.98 (dd, J=6.8, 4.0 Hz, 1H), 4.17-3.88 (m, 2H), 3.63 (s, 3H), 3.44 (d, J=2.8 Hz, 2H), 3.20-3.01 (m, 1H), 3.01-2.83 (m, 1H), 2.77 (dd, J=13.9, 7.0 Hz, 1H), 2.12-1.99 (m, 1H), 1.97-1.72 (m, 2H), 1.57 (t, J=14.0 Hz, 2H). ¹⁹F NMR (282 MHz, DMSO-d₆) δ −112.76; MS (ES+): 556.3 (M+1); (ES−): 554.3 (M−1).

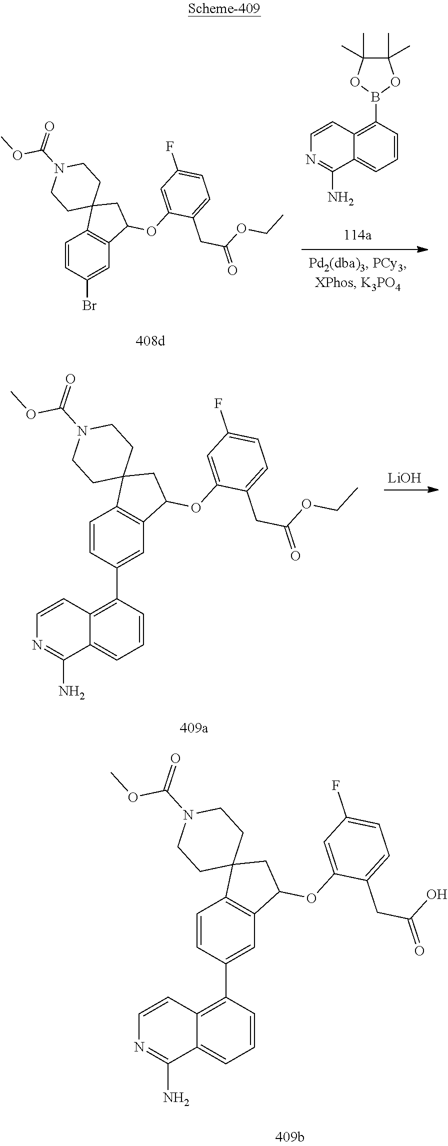

### Preparation of 2-(2-((5-(1-aminoisoquinolin-5-yl)-1′-(methoxycarbonyl)-2,3-dihydrospiro[indene-1,4′-piperidin]-3-yl)oxy)-4-fluorophenyl)acetic acid (409b)

### Step-1: Preparation of methyl 5-(1-aminoisoquinolin-5-yl)-3-(2-(2-ethoxy-2-oxoethyl)-5-fluorophenoxy)-2,3-dihydrospiro[indene-1,4′-piperidine]-1′-carboxylate (409a)

Compound 409a was prepared according to the procedure reported in step-3 of scheme-112, from methyl 5-bromo-3-(2-(2-ethoxy-2-oxoethyl)-5-fluorophenoxy)-2,3-dihydrospiro[indene-1,4′-piperidine]-1′-carboxylate (408d) (250 mg, 0.480 mmol) in dioxane/2-MeTHF (3 mL) using 5-(4,4,5,5-tetramethyl-1,3,2-dioxaborolan-2-yl)isoquinolin-1-amine (114a) (260 mg, 0.961 mmol), K₃PO₄ (3M aqueous solution, 320 μl, 0.961 mmol), PCy₃ (53.9 mg, 0.192 mmol), XPhos (22.90 mg, 0.048 mmol), Pd₂(dba)₃ (88 mg, 0.096 mmol) and heating at 100° C. for 7 h to afford after workup and purification using method-F, methyl 5-(1-aminoisoquinolin-5-yl)-3-(2-(2-ethoxy-2-oxoethyl)-5-fluorophenoxy)-2,3-dihydrospiro[indene-1,4′-piperidine]-1′-carboxylate (409a) (195 mg, 70% yield) as a pale yellow solid; MS (ES+): 584.2 (M+1).

### Step-2: Preparation of 2-(2-((5-(1-aminoisoquinolin-5-yl)-1′-(methoxycarbonyl)-2,3-dihydrospiro[indene-1,4′-piperidin]-3-yl)oxy)-4-fluorophenyl)acetic acid (409b)

Compound 409b was prepared according to the procedure reported in step-2 of scheme-1, from methyl 5-(1-aminoisoquinolin-5-yl)-3-(2-(2-ethoxy-2-oxoethyl)-5-fluorophenoxy)-2,3-dihydrospiro[indene-1,4′-piperidine]-1′-carboxylate (409a) (190 mg, 0.326 mmol) in MeOH (3.2 mL) and THF (3.2 mL) using lithium hydroxide (1 N solution, 1628 μl, 1.628 mmol) and stirring for 28 h at RT to afford after work up and purification using method-G, 2-(2-((5-(1-aminoisoquinolin-5-yl)-1′-(methoxycarbonyl)-2,3-dihydrospiro[indene-1,4′-piperidin]-3-yl)oxy)-4-fluorophenyl)acetic acid (409b) (117 mg, 65% yield) HCl salt as a white solid; ¹H NMR (300 MHz, DMSO-d₆) δ 13.39 (s, 1H, D₂O exchangeable), 12.05 (s, 1H, D₂O) exchangeable), 9.12 (s, 2H, D₂O exchangeable), 8.61 (d, J=8.2 Hz, 1H), 8.03-7.72 (m, 2H), 7.67-7.48 (m, 2H), 7.48-7.36 (m, 2H), 7.31-7.11 (m, 2H), 6.95 (d, J=7.2 Hz, 1H), 6.73 (td, J=8.4, 2.4 Hz, 1H), 5.98 (dd, J=6.8, 4.4 Hz, 1H), 4.19-3.89 (m, 2H), 3.63 (s, 3H), 3.42 (s, 2H), 3.21-3.03 (m, 1H), 3.02-2.88 (m, 1H), 2.83 (dd, J=13.8, 6.9 Hz, 1H), 2.06 (dd, J=13.7, 4.4 Hz, 1H), 1.99-1.71 (m, 2H), 1.62 (t, J=15.1 Hz, 2H); IF NMR (282 MHz, DMSO-d₆) δ −112.82; MS (ES+): 556.3 (M+1); (ES−): 554.3 (M−1).

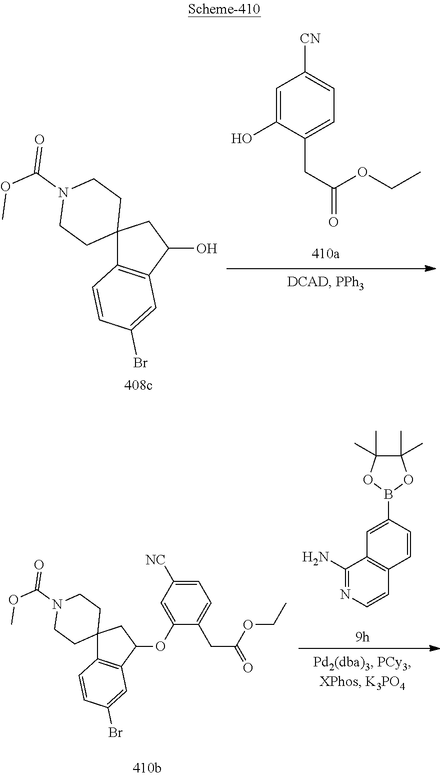 [SMILES: COC(=O)N1CCC2(CC1)CC(O)c1cc(Br)ccc12 ; CCOC(=O)Cc1ccc(C#N)cc1O ; CCOC(=O)Cc1ccc(C#N)cc1OC1CC2(CCN(C(=O)OC)CC2)c2ccc(Br)cc21 ; CC1(C)OB(c2ccc3ccnc(N)c3c2)OC1(C)C]

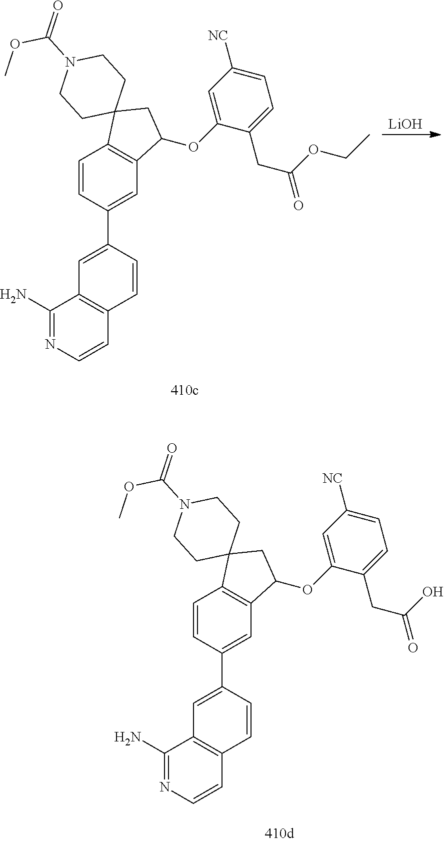

### Preparation of 2-(2-((5-(1-aminoisoquinolin-7-yl)-1′-(methoxycarbonyl)-2,3-dihydrospiro[indene-1,4′-piperidin]-3-yl)oxy)-4-cyanophenyl)acetic acid (410d)

### Step-1: Preparation of methyl 5-bromo-3-(5-cyano-2-(2-ethoxy-2-oxoethyl)phenoxy)-2,3-dihydrospiro[indene-1,4′-piperidine]-1′-carboxylate (410b)

Compound 410b was prepared according to the procedure reported in step-2 of scheme-2, from methyl 5-bromo-3-hydroxy-2,3-dihydrospiro[indene-1,4′-piperidine]-1′-carboxylate (408c) (0.6 g, 1.764 mmol) in DCM (30 mL) using PPh₃ (0.555 g, 2.116 mmol), ethyl 2-(4-cyano-2-hydroxyphenyl)acetate (410a) (0.434 g, 2.116 mmol; CAS #1261647-89-5) and a solution of DCAD (0.777 g, 2.116 mmol) in DCM (10 mL) to afford after workup and purification using method-O, methyl 5-bromo-3-(5-cyano-2-(2-ethoxy-2-oxoethyl)phenoxy)-2,3-dihydrospiro[indene-1,4′-piperidine]-1′-carboxylate (410b) (0.85 g, 91% yield) as a clear gel; MS (ES+): 549.1 (M+Na).

### Step-2: Preparation of methyl 5-(1-aminoisoquinolin-7-yl)-3-(5-cyano-2-(2-ethoxy-2-oxoethyl)phenoxy)-2,3-dihydrospiro[indene-1,4′-piperidine]-1′-carboxylate (410c)

Compound 410c was prepared according to the procedure reported in step-3 of scheme-112, from methyl 5-bromo-3-(5-cyano-2-(2-ethoxy-2-oxoethyl)phenoxy)-2,3-dihydrospiro[indene-1,4′-piperidine]-1′-carboxylate (410b) (0.4 g, 0.758 mmol) in dioxane/2-MeTHF (5 mL) using 7-(4,4,5,5-tetramethyl-1,3,2-dioxaborolan-2-yl)isoquinolin-1-amine (9h) (0.410 g, 1.517 mmol), K₃PO₄ (3M aqueous solution, 0.506 mL, 1.517 mmol), PCy₃ (0.064 g, 0.228 mmol), XPhos (0.036 g, 0.076 mmol). Pd₂(dba)₃ (0.139 g, 0.152 mmol) and heating at 100° C. for 3 h to afford after workup and purification method-BW, methyl 5-(1-aminoisoquinolin-7-yl)-3-(5-cyano-2-(2-ethoxy-2-oxoethyl)phenoxy)-2,3-dihydrospiro[indene-1,4′-piperidine]-1′-carboxylate (410c) (0.16 g, 36% yield) as a pale yellow solid; MS (ES+): 591.3 (M+1).

### Step-3: Preparation of 2-(2-((5-(1-aminoisoquinolin-7-yl)-1′-(methoxycarbonyl)-2,3-dihydrospiro[indene-1,4′-piperidin]-3-yl)oxy)-4-cyanophenyl)acetic acid (410d)

Compound 410d was prepared according to the procedure reported in step-2 of scheme-1, from methyl 5-(1-aminoisoquinolin-7-yl)-3-(5-cyano-2-(2-ethoxy-2-oxoethyl)phenoxy)-2,3-dihydrospiro[indene-1,4′-piperidine]-1′-carboxylate (410c) (0.16 g, 0.271 mmol) in MeOH (2 mL) and THF (2 mL) using lithium hydroxide (1 N solution, 1.083 mL, 1.083 mmol) to afford after work up and purification using method-G, 2-(2-((5-(1-aminoisoquinolin-7-yl)-1′-(methoxycarbonyl)-2,3-dihydrospiro[indene-1,4′-piperidin]-3-yl)oxy)-4-cyanophenyl)acetic acid (410d) (55 mg, 36% yield) HCl salt as a white solid; ¹H NMR (300 MHz, DMSO-d₆) 13.31 (s, 1H, D₂O exchangeable), 12.29 (s, 1H, D₂O exchangeable), 9.16 (s, 2H, D₂O exchangeable), 8.90 (d, J=1.8 Hz, 1H), 8.32 (dd, J=8.5, 1.6 Hz, 1H), 8.04 (d, J=8.5 Hz, 1H), 7.96-7.83 (m, 2H), 7.81 (s, 1H), 7.70 (d, J=6.9 Hz, 1H), 7.58 (d, J=8.0 Hz, 1H), 7.50-7.37 (m, 2H), 7.27 (d, J=6.9 Hz, 1H), 6.07 (dd, J=6.8, 4.2 Hz, 1H), 4.13-3.88 (m, 2H), 3.63 (s, 3H), 3.58 (d, J=2.7 Hz, 2H), 3.23-2.77 (m, 3H), 2.10-1.71 (m, 3H), 1.66-1.38 (m, 2H); MS (ES+): 563.3 (M+1); (ES−): 561.1 (M−1); FT-1R: 2228.9 cm⁻¹.

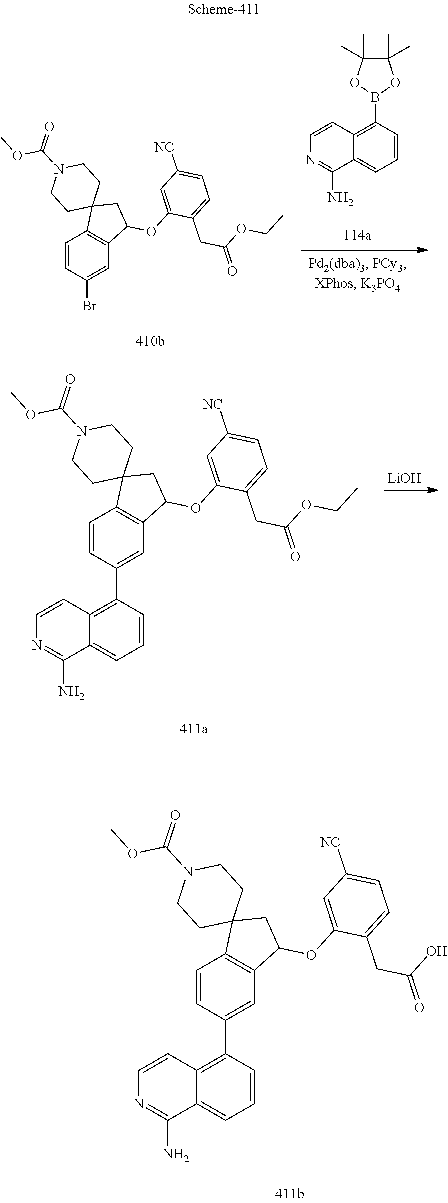

### Preparation of 2-(2-((5-(1-aminoisoquinolin-5-yl)-1′-(methoxycarbonyl)-2,3-dihydrospiro[indene-1,4′-piperidin]-3-yl)oxy)-4-cyanophenyl)acetic acid (411b)

### Step-1: Preparation of methyl 5-(1-aminoisoquinolin-5-yl)-3-(5-cyano-2-(2-ethoxy-2-oxoethyl)phenoxy)-2,3-dihydrospiro[indene-1,4′-piperidine]-1′-carboxylate (411a)

Compound 411a was prepared according to the procedure reported in step-3 of scheme-112, from methyl 5-bromo-3-(5-cyano-2-(2-ethoxy-2-oxoethyl)phenoxy)-2,3-dihydrospiro[indene-1,4′-piperidine]-1′-carboxylate (410b) (0.4 g, 0.758 mmol) in dioxane/2-MeTHF (5 mL) using 5-(4,4,5,5-tetramethyl-1,3,2-dioxaborolan-2-yl)isoquinolin-1-amine (114a) (0.410 g, 1.517 mmol), K₃PO₄ (3M aqueous solution, 0.506 mL, 1.517 mmol), PCy₃ (0.064 g, 0.228 mmol), XPhos (0.036 g, 0.076 mmol), Pd₂(dba)₃ (0.139 g, 0.152 mmol) and beating at 100° C. for 3 h to afford after workup and purification using method-BW, methyl 5-(1-aminoisoquinolin-5-yl)-3-(5-cyano-2-(2-ethoxy-2-oxoethyl)phenoxy)-2,3-dihydrospiro[indene-1,4′-piperidine]-1′-carboxylate (411a) (0.21 g, 47% yield) as a pale yellow solid; MS (ES+): 591.3 (M+1).

### Step-2: Preparation of 2-(2-((5-(1-aminoisoquinolin-5-yl)-1′-(methoxycarbonyl)-2,3-dihydrospiro[indene-1,4′-piperidin]-3-yl)oxy)-4-cyanophenyl)acetic acid (411b)

Compound 411b was prepared according to the procedure reported in step-2 of scheme-1, from methyl 5-(1-aminoisoquinolin-5-yl)-3-(5-cyano-2-(2-ethoxy-2-oxoethyl)phenoxy)-2,3-dihydrospiro[indene-1,4′-piperidine]-1′-carboxylate (411a) (0.2 g, 0.339 mmol) in MeOH (2.5 mL) and THF (2.5 mL) using lithium hydroxide (1 N solution, 1.354 mL, 1.354 mmol) to afford after work up and purification using method-G, 2-(2-((5-(1-aminoisoquinolin-5-yl)-1′-(methoxycarbonyl)-2,3-dihydrospiro[indene-1,4′-piperidin]-3-yl)oxy)-4-cyanophenyl)acetic acid (411b) (123 mg, 65% yield) HCl salt as a white solid; ¹H NMR (300 MHz, DMSO-d₆) δ 13.49 (s, 1H, D₂O exchangeable), 12.23 (s, 1H, D₂O exchangeable), 9.24 (s, 2H, D₂O exchangeable), 8.64 (d, J=8.2 Hz, 1H), 7.96-7.74 (m, 3H), 7.67-7.51 (m, 2H), 7.49-7.35 (m, 4H), 6.96 (d, J=7.2 Hz, 1H), 6.05 (dd, J=6.8, 4.6 Hz, 1H), 4.20-3.92 (m, 2H), 3.63 (s, 3H), 3.59-3.52 (m, 2H), 3.28-2.77 (m, 3H), 2.15-1.86 (m, 2H), 1.86-1.70 (m, 1H), 1.70-1.51 (m, 2H); MS (ES+): 563.3 (M+1); (ES−): 1123.3 (2M−1); FT-1R: 2228.9 cm⁻¹.

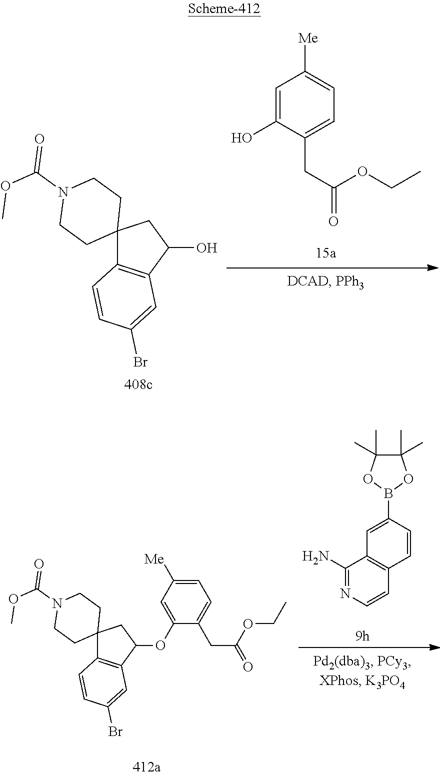 [SMILES: COC(=O)N1CCC2(CC1)CC(O)c1cc(Br)ccc12 ; CCOC(=O)Cc1ccc(C)cc1O ; CCOC(=O)Cc1ccc(C)cc1OC1CC2(CCN(C(=O)OC)CC2)c2ccc(Br)cc21 ; CC1(C)OB(c2ccc3ccnc(N)c3c2)OC1(C)C]

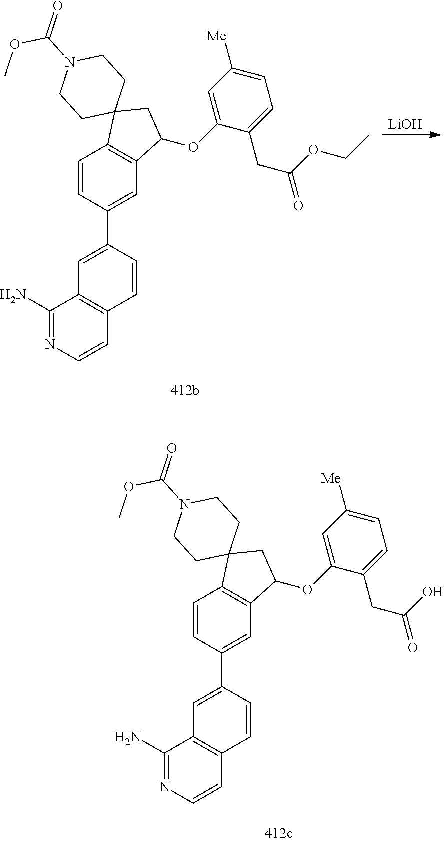

### Preparation of 2-(2-((5-(1-aminoisoquinolin-7-yl)-1′-(methoxycarbonyl)-2,3-dihydrospiro[indene-1,4′-piperidin]-3-yl)oxy)-4-methylphenyl)acetic acid (412c)

### Step-1: Preparation of methyl 5-bromo-3-(2-(2-ethoxy-2-oxoethyl)-5-methylphenoxy)-2,3-dihydrospiro[indene-1,4′-piperidine]-1′-carboxylate (412a)

Compound 412a was prepared according to the procedure reported in step-2 of scheme-2, from methyl 5-bromo-3-hydroxy-2,3-dihydrospiro[indene-1,4′-piperidine]-1′-carboxylate (408c) (0.6 g, 1.764 mmol) in DCM (30 mL) using PPh₃ (0.555 g, 2.116 mmol), ethyl 2-(2-hydroxy-4-methylphenyl)acetate (15a) (0.411 g, 2.116 mmol) and a solution of DCAD (0.777 g, 2.116 mmol) in DCM (10 mL) to afford after workup and purification using method-O, methyl 5-bromo-3-(2-(2-ethoxy-2-oxoethyl)-5-methylphenoxy)-2,3-dihydrospiro[indene-1,4′-piperidine]-1′-carboxylate (412a) (0.62 g, 68% yield) as a clear gel; MS (ES+): 538.1 (M+Na).

### Step-2: Preparation of methyl 5-(1-aminoisoquinolin-7-yl)-3-(2-(2-ethoxy-2-oxoethyl)-5-methylphenoxy)-2,3-dihydrospiro[indene-1,4′-piperidine]-1′-carboxylate (412b)

Compound 412b was prepared according to the procedure reported in step-3 of scheme-112, from methyl 5-bromo-3-(2-(2-ethoxy-2-oxoethyl)-5-methylphenoxy)-2,3-dihydrospiro[indene-1,4′-piperidine]-1′-carboxylate (412a) (0.3 g, 0.581 mmol) in dioxane/2-MeTHF (4 mL) using 7-(4,4,5,5-tetramethyl-1,3,2-dioxaborolan-2-yl)isoquinolin-1-amine (9h) (0.314 g, 1.162 mmol), K₃PO₄ (3M aqueous solution, 0.387 mL, 1.162 mmol), PCy₃ (0.049 g, 0.174 mmol), XPhos (0.028 g, 0.058 mmol), Pd₂(dba)₃ (0.106 g, 0.116 mmol) and heating at 100° C. for 2 h to afford after workup and purification method-BW, methyl 5-(1-aminoisoquinolin-7-yl)-3-(2-(2-ethoxy-2-oxoethyl)-5-methylphenoxy)-2,3-dihydrospiro[indene-1,4′-piperidine]-1′-carboxylate (412b) (0.1g, 30% yield) as a pale yellow solid; MS (ES+): 580.3 (M+1).

### Step-3: Preparation of 2-(2-((5-(1-aminoisoquinolin-7-yl)-1′-(methoxycarbonyl)-2,3-dihydrospiro[indene-1,4′-piperidin]-3-yl)oxy)-4-methylphenyl)acetic acid (412c)

Compound 412c was prepared according to the procedure reported in step-2 of scheme-1, from methyl 5-(1-aminoisoquinolin-7-yl)-3-(2-(2-ethoxy-2-oxoethyl)-5-methylphenoxy)-2,3-dihydrospiro[indene-1,4′-piperidine]-1′-carboxylate (412b) (0.1 g, 0.173 mmol) in MeOH (1.5 mL) and THF (1.5 mL) using lithium hydroxide (1N solution, 0.69 mL, 0.69 mmol) to afford after work up and purification using method-G, 2-(2-((5-(1-aminoisoquinolin-7-yl)-1′-(methoxycarbonyl)-2,3-dihydrospiro[indene-1,4′-piperidin]-3-yl)oxy)-4-methylphenyl)acetic acid (412c) (48 mg, 50% yield) HCl salt as a white solid; ¹H NMR (300 MHz, DMSO-d₆) δ 13.41 (s, 1H, D₂O exchangeable), 12.08 (s, 1H, D₂O exchangeable), 9.22 (s, 3H, D₂O exchangeable), 8.92 (d, J=1.7 Hz, 1H), 8.32 (dd, J=8.5, 1.6 Hz, 1H), 8.04 (d, J=8.5 Hz, 1H), 7.95-7.84 (m, 2H), 7.71 (d, J=6.9 Hz, 1H), 7.57 (d, J=8.0 Hz, 1H), 7.27 (d, J=7.0 Hz, 1H), 7.16-7.02 (m, 2H), 6.79-6.68 (m, 1H), 5.94 (dd, J=6.9, 4.1 Hz, 1H), 4.18-3.92 (m, 2H), 3.64 (d, J=1.9 Hz, 3H), 3.42 (d, J=3.5 Hz, 2H), 3.21-3.04 (m, 1H), 3.01-2.85 (m, 1H), 2.75 (dd, J=13.8, 6.9 Hz, 1H), 2.37 (s, 3H), 2.14-2.01 (m, 1H), 1.98-1.75 (m, 2H), 1.67-1.49 (m, 2H); MS (ES+): 552.3 (M+1); (ES−): 550.3 (M−1).

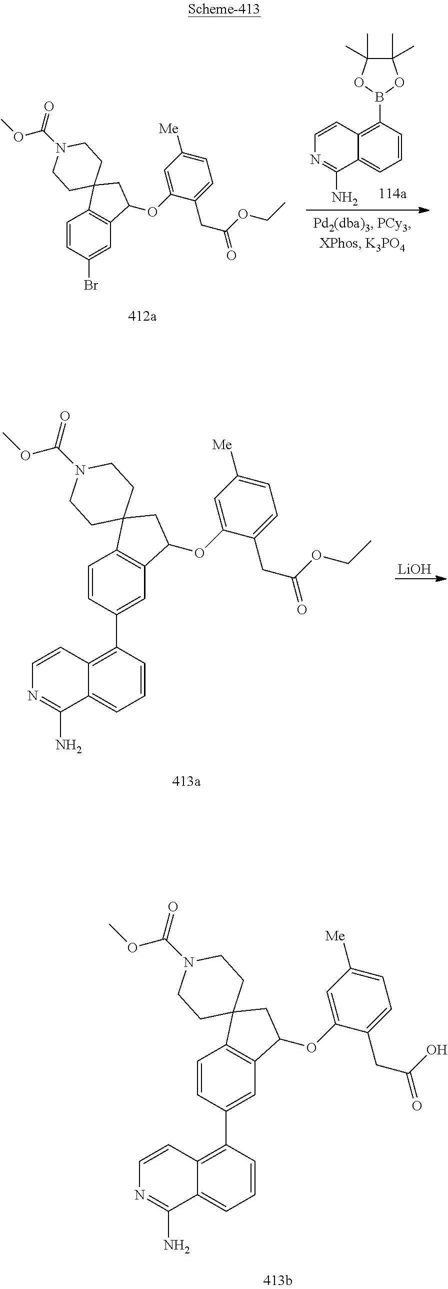

### Preparation of 2-(2-((5-(1-aminoisoquinolin-5-yl)-1′-(methoxycarbonyl)-2,3-dihydrospiro[indene-1,4′-piperidin]-3-yl)oxy)-4-methylphenyl)acetic acid (413b)

### Step-1: Preparation of methyl 5-(1-aminoisoquinolin-5-yl)-3-(2-(2-ethoxy-2-oxoethyl)-5-methylphenoxy)-2,3-dihydrospiro[indene-1,4′-piperidine]-1′-carboxylate (413a)

Compound 413a was prepared according to the procedure reported in step-3 of scheme-112, from methyl 5-bromo-3-(2-(2-ethoxy-2-oxoethyl)-5-methylphenoxy)-2,3-dihydrospiro[indene-1,4′-piperidine]-1′-carboxylate (412a) (0.3 g, 0.581 mmol) in dioxane/2-MeTHF (4 mL) using 5-(4,4,5,5-tetramethyl-1,3,2-dioxaborolan-2-yl)isoquinolin-1-amine (114a) (0.314 g, 1.162 mmol). K₃PO₄ (3M aqueous solution, 0.387 mL, 1.162 mmol), PCy₃ (0.049 g, 0.174 mmol), XPhos (0.028 g, 0.058 mmol), Pd₂(dba)₃ (0.106 g, 0.116 mmol) and heating at 100° C. for 3 h to afford after workup and purification using method-BW, methyl 5-(1-aminoisoquinolin-5-yl)-3-(2-(2-ethoxy-2-oxoethyl)-5-methylphenoxy)-2,3-dihydrospiro[indene-1,4′-piperidine]-1′-carboxylate (413a) (0.07 g, 21% yield) as a pale yellow solid; MS (ES+): 580.3 (M+1).

### Step-2: Preparation of 2-(2-((5-(1-aminoisoquinolin-5-yl)-1′-(methoxycarbonyl)-2,3-dihydrospiro[indene-1,4′-piperidin]-3-yl)oxy)-4-methylphenyl)acetic acid (413b)

Compound 413b was prepared according to the procedure reported in step-2 of scheme-1, from methyl 5-(1-aminoisoquinolin-5-yl)-3-(2-(2-ethoxy-2-oxoethyl)-5-methylphenoxy)-2,3-dihydrospiro[indene-1,4′-piperidine]-1′-carboxylate (413a) (0.07 g, 0.121 mmol) in MeOH (1 mL) and THF (1 mL) using lithium hydroxide (1N solution, 0.483 mL, 0.483 mmol) to afford after work up and purification using method-G, 2-(2-((5-(1-aminoisoquinolin-5-yl)-1′-(methoxycarbonyl)-2,3-dihydrospiro[indene-1,4′-piperidin]-3-yl)oxy)-4-methylphenyl)acetic acid (413b) (25 mg, 38% yield) HCl salt as a white solid; ¹H NMR (300 MHz, DMSO-d₆) δ 13.36 (s, 1H, D₂O exchangeable), 11.98 (s, 1H, D₂O exchangeable), 9.19 (s, 2H, D₂O exchangeable), 8.62 (d, J=8.2 Hz, 1H), 7.95-7.79 (m, 2H), 7.65-7.50 (m, 2H), 7.47-7.37 (m, 2H), 7.14-7.00 (m, 2H), 6.97 (d, J=7.3 Hz, 1H), 6.71 (d, J=7.6 Hz, 1H), 5.92 (dd, J==6.8, 4.5 Hz, 1H), 4.15-3.91 (m, 2H), 3.63 (s, 3H), 3.38 (s, 2H), 3.17-2.87 (m, 2H), 2.80 (dd, J=13.7, 7.0 Hz, 1H), 2.32 (s, 3H), 2.15-2.02 (m, 1H), 2.00-1.72 (m, 2H), 1.72-1.53 (m, 2H); MS (ES+): 552.3 (M+1); (ES−): 550.3 (M−1).

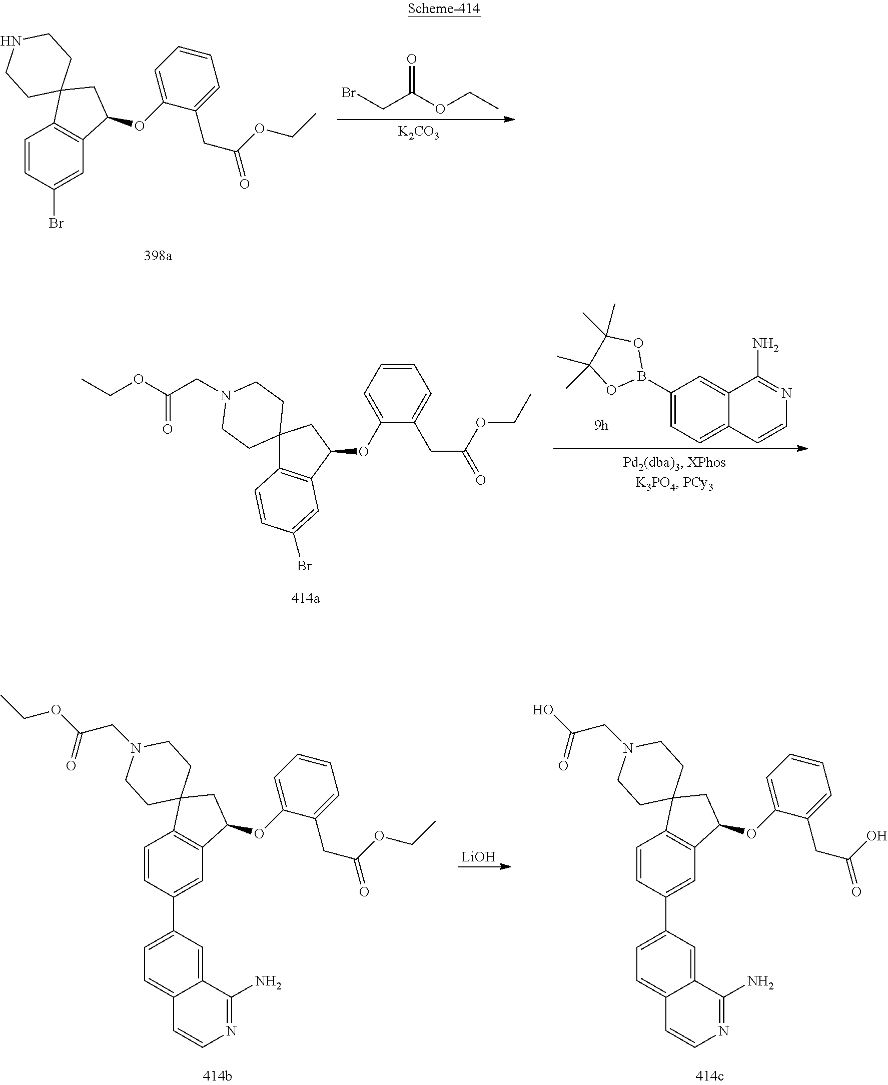

### Preparation of (R)-2-(2-((5-(1-aminoisoquinolin-7-yl)-1′-(carboxymethyl)-2,3-dihydrospiro[indene-1,4′-piperidin]-3-yl)oxy)phenyl)acetic acid (414c)

### Step-1: Preparation of (R)-ethyl 2-(2-((5-bromo-1′-(2-ethoxy-2-oxoethyl)-2,3-dihydrospiro[indene-1,4′-piperidin]-3-yl)oxy)phenyl)acetate (414a)

Compound 414a was prepared according to the procedure reported in step-2 of scheme-297, from (R)-ethyl 2-(2-((5-bromo-2,3-dihydrospiro[indene-1,4′-piperidin]-3-yl)oxy)phenyl)acetate (398a) (600 mg, 1.350 mmol) in DMF (5 mL) using ethyl 2-bromoacetate (271 mg, 1.620 mmol) and K₂CO₃ (560 mg, 4.05 mmol) stirring for 4 h at 50° C. to afford after work up and purification using method-P, (R)-ethyl 2-(2-((5-bromo-1′-(2-ethoxy-2-oxoethyl)-2,3-dihydrospiro[indene-1,4′-piperidin]-3-yl)oxy)phenyl)acetate (414a) (400 mg, 56% yield) as a yellow oil; MS (ES+) 530.1 and 532.1 (M+1).

### Step-2: Preparation of (R)-ethyl 2-(2-((5-(1-aminoisoquinolin-7-yl)-1′-(2-ethoxy-2-oxoethyl)-2,3-dihydrospiro[indene-1,4′-piperidin]-3-yl)oxy)phenyl)acetate (414b)

Compound 414b was prepared according to the procedure reported in step-3 of scheme-112, from (R)-ethyl 2-(2-((5-bromo-1′-(2-ethoxy-2-oxoethyl)-2,3-dihydrospiro[indene-1,4′-piperidin]-3-yl)oxy)phenyl)acetate (414a) (200 mg, 0.377 mmol) in dioxane/2-MeTHF (2 mL) using 7-(4,4,5,5-tetramethyl-1,3,2-dioxaborolan-2-yl)isoquinolin-1-amine (9h) (204 mg, 0.754 mmol), K₃PO₄ (3M aqueous solution, 251 μl, 0.754 mmol), PCy₃ (42.3 mg, 0.151 mmol), XPhos (35.9 mg, 0.075 mmol), Pd₂(dba)₃ (69.1 mg, 0.075 mmol) and heating at 100° C. for 10 b to afford after workup and purification using method-U. (R)-ethyl 2-(2-((5-(1-aminoisoquinolin-7-yl)-1′-(2-ethoxy-2-oxoethyl)-2,3-dihydrospiro[indene-1,4′-piperidin]-3-yl)oxy)phenyl)acetate (414b) (63 mg, 28% yield) as a clear oil; MS (ES+): 594.4 (M+1); (ES−) 592.3 (M−1).

### Step-3: Preparation of (R)-2-(2-((5-(1-aminoisoquinolin-7-yl)-1′-(carboxymethyl)-2,3-dihydrospiro[indene-1,4′-piperidin]-3-yl)oxy)phenyl)acetic acid (414c)

Compound 414c was prepared according to the procedure reported in step-2 of scheme-1, from (R)-ethyl 2-(2-(5-(1-aminoisoquinolin-7-yl)-J′-(2-ethoxy-2-oxoethyl)-2,3-dihydrospiro[indene-1,4′-piperidin]-3-yl)oxy)phenyl)acetate (414b) (63 mg, 0.106 mmol) in THF (3 mL) using lithium hydroxide (27.1 mg, 1.131 mmol) in water (1 mL) to afford after work up and purification using method-M, (R)-2-(2-((5-(1-aminoisoquinolin-7-yl)-1′-(carboxymethyl)-2,3-dihydrospiro[indene-1,4′-piperidin]-3-yl)oxy)phenyl)acetic acid (414c) (15 mg, 7% yield) HCl salt as a white solid; ¹H NMR (300 MHz, DMSO-d₆) δ 12.09 (s, 1H, D₂O exchangeable), 9.09 (s, 2H, D₂O exchangeable), 8.93 (s, 1H), 8.32 (dd, J=8.5, 1.6 Hz, 1H), 8.10-7.90 (m, 3H), 7.72 (d, J=6.9 Hz, 1H), 7.49 (d, J=7.9 Hz, 1H), 7.33 (d, J=4.1 Hz, 2H), 7.25 (dd, J=10.9, 7.1 Hz, 2H), 7.01-6.88 (m, 1H), 5.99 (dd, J=6.7, 3.7 Hz, 1H), 4.07 (s, 2H), 3.50-3.44 (m, 4H), 3.18-3.07 (m, 2H), 2.79-2.67 (m, 1H), 2.41-2.24 (m, 2H), 2.15 (dd, J=13.8, 3.6 Hz, 1H), 1.79 (t, J=14.9 Hz, 2H); MS (ES+): 538.2 (M+1); (ES−): 536.2 (M−1).

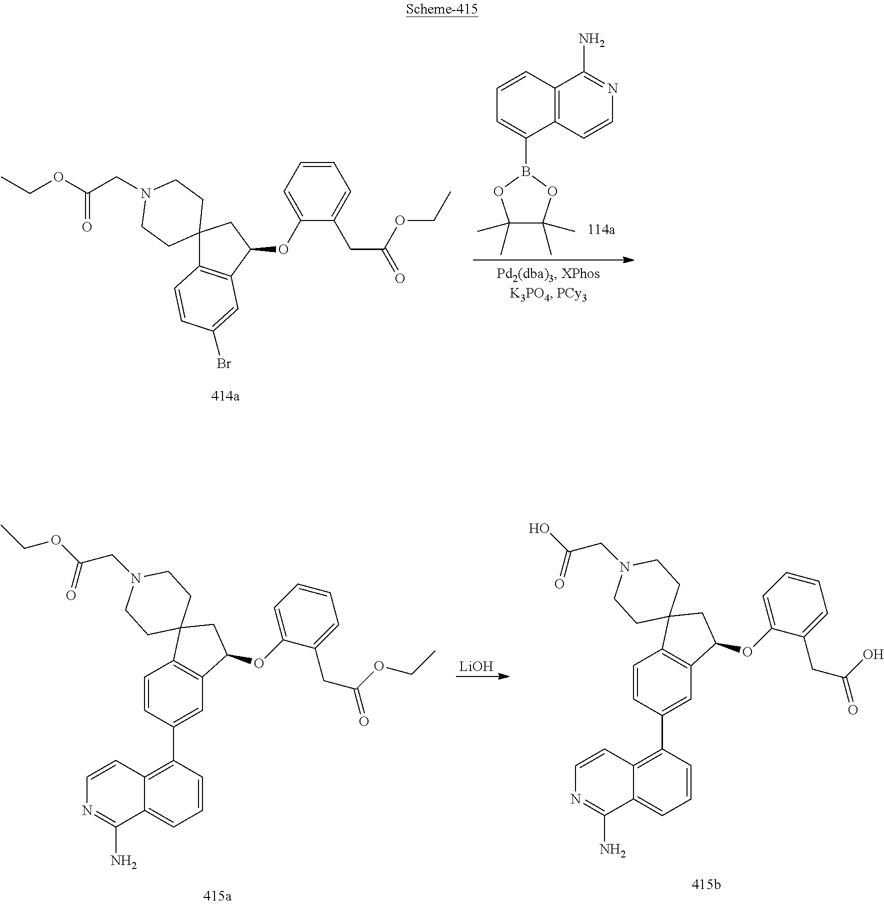

### Preparation of (R)-2-(2-((5-(1-aminoisoquinolin-5-yl)-1′-(carboxymethyl)-2,3-dihydrospiro[indene-1,4′-piperidin]-3-yl)oxy)phenyl)acetic acid (415b)

### Step-1: Preparation of (R)-ethyl 2-(2-((5-(1-aminoisoquinolin-5-yl)-1′-(2-ethoxy-2-oxoethyl)-2,3-dihydrospiro[indene-1,4′-piperidin]-3-yl)oxy)phenyl)acetate (415a)

Compound 415a was prepared according to the procedure reported in step-3 of scheme-112, from (R)-ethyl 2-(2-((5-bromo-1′-(2-ethoxy-2-oxoethyl)-2,3-dihydrospiro[indene-1,4′-piperidin]-3-yl)oxy)phenyl)acetate (414a) (200 mg, 0.377 mmol) in dioxane/2-MeTHF (2 mL) using 5-(4,4,5,5-tetramethyl-1,3,2-dioxaborolan-2-yl)isoquinolin-1-amine (114a) (204 mg, 0.754 mmol), K₃PO₄ (3M aqueous solution, 251 μl, 0.754 mmol), PCy₃ (42.3 mg, 0.151 mmol), XPhos (35.9 mg, 0.075 mmol), Pd₂(dba)₃ (69.1 mg, 0.075 mmol) and heating at 100° C. for 10 h to afford after workup and purification using method-U, (R)-ethyl 2-(2-((5-(1-aminoisoquinolin-5-yl)-1′-(2-ethoxy-2-oxoethyl)-2,3-dihydrospiro[indene-1,4′-piperidin]-3-yl)oxy)phenyl)acetate (415a) (103 mg, 46% yield) as a clear oil; MS (ES+): 594.3 (M+1); (ES−): 592.3 (M−1).

### Step-2: Preparation of (R)-2-(2-((5-(1-aminoisoquinolin-5-yl)-1′-(carboxymethyl)-2,3-dihydrospiro[indene-1,4′-piperidin]-3-yl)oxy)phenyl)acetic acid (415b)

Compound 415b was prepared according to the procedure reported in step-2 of scheme-1, from (R)-ethyl 2-(2-((5-(1-aminoisoquinolin-5-yl)-1′-(2-ethoxy-2-oxoethyl)-2,3-dihydrospiro[indene-1,4′-piperidin]-3-yl)oxy)phenyl)acetate (415a) (103 mg, 0.173 mmol) in THF (3 mL) using lithium hydroxide (27.1 mg, 1.131 mmol) in water (1 mL) to afford after work up and purification using method-M, (R)-2-(2-((5-(1-aminoisoquinolin-5-yl)-1′-(carboxymethyl)-2,3-dihydrospiro[indene-1,4′-piperidin]-3-yl)oxy)phenyl)acetic acid (415b) (76 mg, 38% yield) HCl salt as a white solid; H NMR (300 MHz, DMSO-d₆) δ 12.08 (s, 2H, D₂O exchangeable), 9.08 (s, 2H, D₂O exchangeable), 8.92 (s, 1H), 8.31 (dd, J=8.5, 1.6 Hz, 1H), 8.09-7.87 (m, 3H), 7.71 (d, J=6.9 Hz, 1H), 7.49 (d, J=7.9 Hz, 1H), 7.32 (d, J=4.1 Hz, 2H), 7.29-7.18 (m, 2H), 7.00-6.87 (m, 1H), 6.04-5.92 (m, 1H), 4.07 (s, 2H), 3.55-3.42 (m, 4H), 3.19-3.07 (m, 2H), 2.80-2.66 (m, 1H), 2.40-2.25 (m, 2H), 2.14 (dd, J=13.9, 3.6 Hz, 1H), 1.78 (t, J=14.9 Hz, 2H); MS (ES+): 538.2 (M+1); (ES−): 536.2 (M−1).

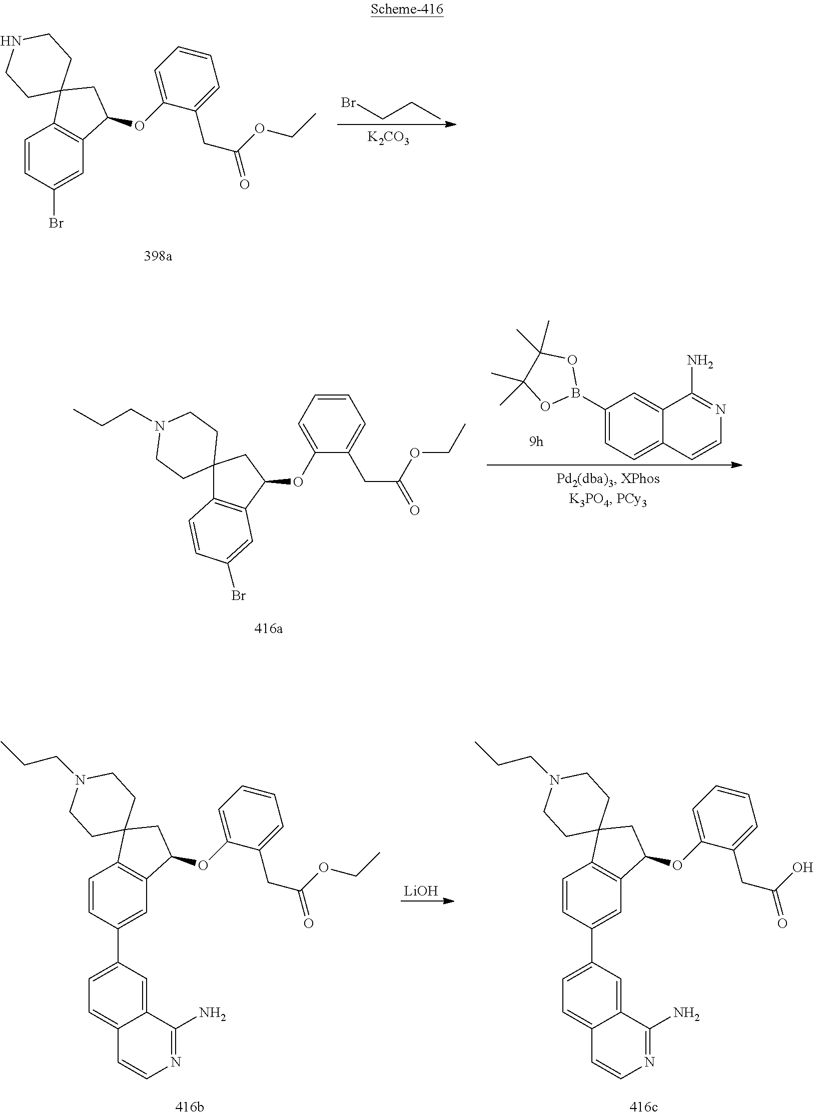

### Preparation of (R)-2-(2-((5-(1-aminoisoquinolin-7-yl)-1′-propyl-2,3-dihydrospiro[indene-1,4′-piperidin]-3-yl)oxy)phenyl)acetic acid (416c)

### Step-1: Preparation of (R)-ethyl 2-(2-((5-bromo-1′-propyl-2,3-dihydrospiro[indene-1,4′-piperidin]-3-yl)oxy)phenyl)acetate (416a)

Compound 416a was prepared according to the procedure reported in step-2 of scheme-297, from (R)-ethyl 2-(2-((5-bromo-2,3-dihydrospiro[indene-1,4′-piperidin]-3-yl)oxy)phenyl)acetate (398a) (600 mg, 1.350 mmol) in DMF (5 ml) using 1-bromopropane (199 mg, 1.620 mmol) and K₂CO₃ (560 mg, 4.05 mmol) stirring for 2 h at 50° C. to afford after work up and purification using method-P, (R)-ethyl 2-(2-((5-bromo-1′-propyl-2,3-dihydrospiro[indene-1,4′-piperidin]-3-yl)oxy)phenyl)acetate (416a) (300 mg, 0.617 mmol, 45.7% yield) as a yellow oil; MS (ES+): 486.1 and 488.1 (M+1); (ES−): 484.0 and 486.0 (M−1).

### Step-2: Preparation of (R)-ethyl 2-(2-((5-(1-aminoisoquinolin-7-yl)-1′-propyl-2,3-dihydrospiro[indene-1,4′-piperidin]-3-yl)oxy)phenyl)acetate (416b)

Compound 416b was prepared according to the procedure reported in step-3 of scheme-112, from (R)-ethyl 2-(2-((5-bromo-1′-propyl-2,3-dihydrospiro[indene-1,4′-piperidin]-3-yl)oxy)phenyl)acetate (416a) (200 mg, 0.377 mmol) in dioxane/2-MeTHF (2 mL) using 7-(4,4,5,5-tetramethyl-1,3,2-dioxaborolan-2-yl)isoquinolin-1-amine (9h) (167 mg, 0.617 mmol), K₃PO₄ (3M aqueous solution, 206 μl, 0.617 mmol), PCy₃ (34.6 mg, 0.123 mmol), XPhos (29.4 mg, 0.062 mmol) and Pd₂(dba)₃ (56.5 mg, 0.062 mmol) and heating at 100° C. for 10 h to afford after workup and purification using method-U. (R)-ethyl 2-(2-((5-(1-aminoisoquinolin-7-yl)-1′-propyl-2,3-dihydrospiro[indene-1,4′-piperidin]-3-yl)oxy)phenyl)acetate (416b) (65 mg, 38% yield) as a clear oil; MS (ES+): 550.3 (M+1).

### Step-3: Preparation of (R)-2-(2-((5-(1-aminoisoquinolin-7-yl)-1′-propyl-2,3-dihydrospiro[indene-1,4′-piperidin]-3-yl)oxy)phenyl)acetic acid (416c)

Compound 416c was prepared according to the procedure reported in step-2 of scheme-1, from (R)-ethyl 2-(2-((5-(1-aminoisoquinolin-7-yl)-1′-propyl-2,3-dihydrospiro[indene-1,4′-piperidin]-3-yl)oxy)phenyl)acetate (416b) (65 mg, 0.118 mmol) in THF (3 mL) using lithium hydroxide (22.15 mg, 0.925 mmol) in water (1 mL) to afford after work up and purification using method-M, (R)-2-(2-((5-(1-aminoisoquinolin-7-yl)-1′-propyl-2,3-dihydrospiro[indene-1,4′-piperidin]-3-yl)oxy)phenyl)acetic acid (416c) (22 mg, 14% yield) HCl salt as a white solid; ¹H NMR (300 MHz, DMSO-d₆) δ 13.42 (s, 1H, D₂O exchangeable), 12.02 (s, 1H, D₂O exchangeable), 10.63 (s, 1H, D₂O exchangeable), 9.17 (s, 2H, D₂O exchangeable), 8.88 (d, J=1.6 Hz, 1H), 8.26 (dd, J=8.5, 1.6 Hz, 1H), 8.02-7.89 (m, 2H), 7.89-7.81 (m, 1H), 7.64 (d, J=6.9 Hz, 1H), 7.36 (d, J=8.0 Hz, 1H), 7.29-7.23 (m, 2H), 7.23-7.13 (m, 2H), 6.94-6.80 (m, 1H), 5.98-5.86 (m, 1H), 3.51-3.32 (m, 4H), 3.15-3.03 (m, 1H), 3.03-2.88 (m, 3H), 2.77-2.62 (m, 1H), 2.40-2.30 (m, 2H), 2.07 (dd, J=14.0, 3.9 Hz, 1H), 1.81-1.61 (m, 4H), 0.86 (t, J=7.4 Hz, 3H); MS (ES+): 522.3 (M+1); (ES−): 520.3 (M−1).

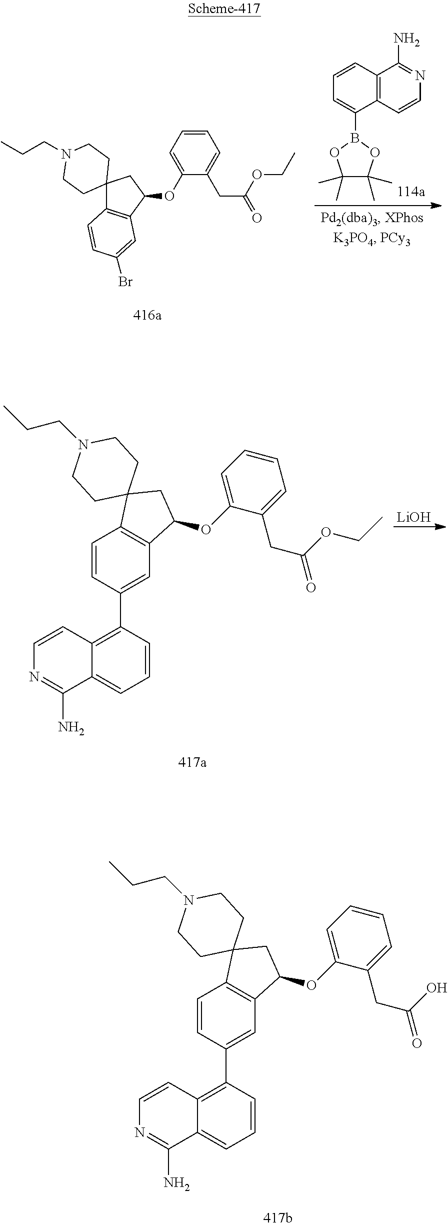

### Preparation of (R)-2-(2-((5-(1-aminoisoquinolin-5-yl)-1′-propyl-2,3-dihydrospiro[indene-1,4′-piperidin]-3-yl)oxy)phenyl)acetic acid (417b)

### Step-1: Preparation of (R)-ethyl 2-(2-((5-(1-aminoisoquinolin-5-yl)-1′-propyl-2,3-dihydrospiro[indene-1,4′-piperidin]-3-yl)oxy)phenyl)acetate (417a)

Compound 417a was prepared according to the procedure reported in step-3 of scheme-112, from (R)-ethyl 2-(2-((5-bromo-1′-propyl-2,3-dihydrospiro[indene-1,4′-piperidin]-3-yl)oxy)phenyl)acetate (416a) (150 mg, 0.308 mmol) in dioxane/2-MeTHF (2 mL) using 5-(4,4,5,5-tetramethyl-1,3,2-dioxaborolan-2-yl)isoquinolin-1-amine (114a) (167 mg, 0.617 mmol), K₃PO₄ (3M aqueous solution, 206 μl, 0.617 mmol), PCy₃ (34.6 mg, 0.123 mmol), XPhos (29.4 mg, 0.062 mmol), Pd₂(dba)₃ (56.5 mg, 0.062 mmol) and heating at 100° C. for 10 b to afford after workup and purification using method-U. (R)-ethyl 2-(2-((5-(1-aminoisoquinolin-5-yl)-1′-propyl-2,3-dihydrospiro[indene-1,4′-piperidin]-3-yl)oxy)phenyl)acetate (417a) (102 mg, 60% yield) as a clear oil; MS (ES+): 550.3 (M+1).

### Step-2: Preparation of (R)-2-(2-((5-(1-aminoisoquinolin-5-yl)-1′-propyl-2,3-dihydrospiro[indene-1,4′-piperidin]-3-yl)oxy)phenyl)acetic acid (417b)

Compound 417b was prepared according to the procedure reported in step-2 of scheme-1, from (R)-ethyl 2-(2-((5-(1-aminoisoquinolin-5-yl)-1′-propyl-2,3-dihydrospiro[indene-1,4′-piperidin]-3-yl)oxy)phenyl)acetate (417a) (102 mg, 0.186 mmol) in THF (3 mL) using lithium hydroxide (22.15 mg, 0.925 mmol) in water (1 mL) to afford after work up and purification using method-M, (R)-2-(2-((5-(1-aminoisoquinolin-5-yl)-1′-propyl-2,3-dihydrospiro[indene-1,4′-piperidin]-3-yl)oxy)phenyl)acetic acid (417b) (73 mg, 45% yield) HCl salt as a white solid; ¹H NMR (300 MHz, DMSO-d₆) δ 13.55 (s, 1H, D₂O exchangeable), 12.00 (s, 1H, D₂O exchangeable), 10.75 (s, 1H, D₂O exchangeable), 9.25 (s, 2H, D₂O exchangeable), 8.66 (d, J=8.2 Hz, 1H), 7.96-7.89 (m, 1H), 7.85 (t, J=7.8 Hz, 1H), 7.64 (d, J=7.3 Hz, 1H), 7.57-7.49 (m, 1H), 7.50-7.40 (m, 2H), 7.33-7.26 (m, 2H), 7.24-7.16 (m, 1H), 7.02-6.85 (m, 2H), 6.03-5.92 (m, 1H), 3.61-3.42 (m, 4H), 3.25-3.13 (m, 2H), 3.09-3.01 (m, 2H), 2.85 (dd, J=13.8, 6.9 Hz, 1H), 2.63-2.54 (m, 1H), 2.48-2.38 (m, 1H), 2.15 (dd, J=13.8, 4.4 Hz, 1H), 1.92-1.70 (m, 4H), 0.95 (t, J=7.4 Hz, 3H); MS (ES+): 522.3 (M+1); (ES−): 520.4 (M−1).

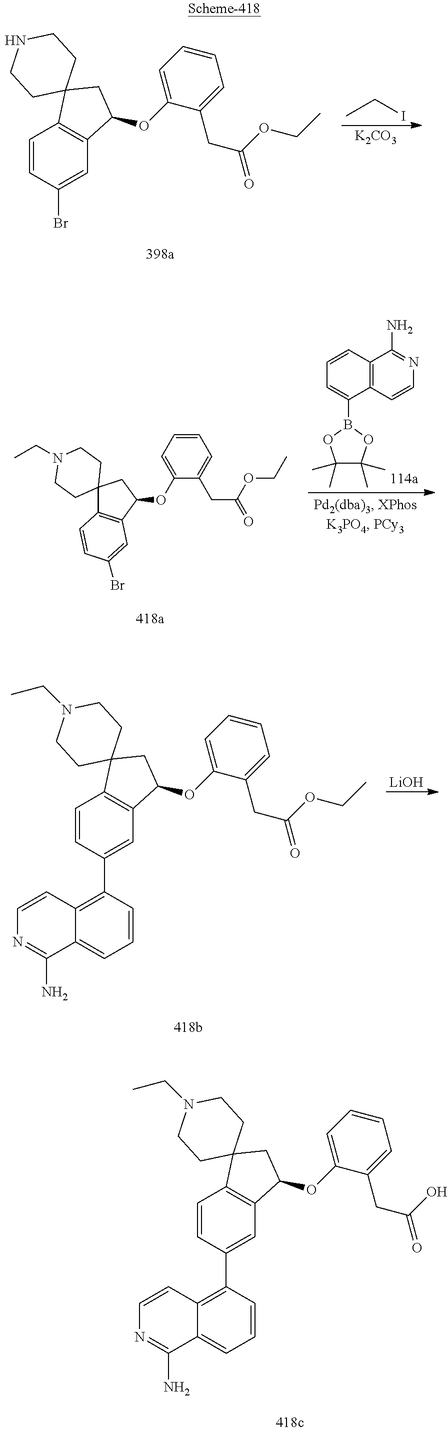

### Preparation of (R)-2-(2-((5-(1-aminoisoquinolin-5-yl)-1′-ethyl-2,3-dihydrospiro[indene-1,4′-piperidin]-3-yl)oxy)phenyl)acetic acid (418c)

### Step-1: Preparation of (R)-ethyl 2-(2-((5-bromo-1′-ethyl-2,3-dihydrospiro[indene-1,4′-piperidin]-3-yl)oxy)phenyl)acetate (418a)

Compound 418a was prepared according to the procedure reported in step-2 of scheme-297, from (R)-ethyl 2-(2-((5-bromo-2,3-dihydrospiro[indene-1,4′-piperidin]-3-yl)oxy)phenyl)acetate (398a) (600 mg, 1.350 mmol) in DMF (5 mL) using iodoethane (253 mg, 1.620 mmol) and K₂CO₃ (560 mg, 4.05 mmol) stirring for 2 h at 50° C. to afford after work up and purification using method-P, (R)-ethyl 2-(2-((5-bromo-1′-ethyl-2,3-dihydrospiro[indene-1,4′-piperidin]-3-yl)oxy)phenyl)acetate (418a) (300 mg, 47% yield) as a yellow oil; MS (ES+): 472.2 (M+1).

### Step-2: Preparation of (R)-ethyl 2-(2-((5-(1-aminoisoquinolin-5-yl)-1′-ethyl-2,3-dihydrospiro[indene-1,4′-piperidin]-3-yl)oxy)phenyl)acetate (418b)

Compound 418b was prepared according to the procedure reported in step-3 of scheme-112, from (R)-ethyl 2-(2-((5-bromo-1′-ethyl-2,3-dihydrospiro[indene-1,4′-piperidin]-3-yl)oxy)phenyl)acetate (418a) (150 mg, 0.318 mmol) in dioxane/2-MeTHF (2 mL) using 5-(4,4,5,5-tetramethyl-1,3,2-dioxaborolan-2-yl)isoquinolin-1-amine (114a) (172 mg, 0.635 mmol), K₃PO₄ (3M aqueous solution, 212 μl, 0.635 mmol), PCy₃ (35.6 mg, 0.127 mmol), XPhos (30.3 mg, 0.064 mmol) and Pd₂(dba)₃ (58.2 mg, 0.064 mmol) and heating at 100° C. for 10 h to afford after workup and purification using method-U, (R)-ethyl 2-(2-((5-(1-aminoisoquinolin-5-yl)-1′-ethyl-2,3-dihydrospiro[indene-1,4′-piperidin]-3-yl)oxy)phenyl)acetate (418b) (62 mg, 37% yield) as a clear oil; MS (ES+): 536.3 (M+1).

### Step-3: Preparation of (R)-2-(2-((5-(1-aminoisoquinolin-5-yl)-1′-ethyl-2,3-dihydrospiro[indene-1,4′-piperidin]-3-yl)oxy)phenyl)acetic acid (418c)

Compound 418c was prepared according to the procedure reported in step-2 of scheme-1, from (R)-ethyl 2-(2-((5-(1-aminoisoquinolin-5-yl)-1′-ethyl-2,3-dihydrospiro[indene-1,4′-piperidin]-3-yl)oxy)phenyl)acetate (418b) (62 mg, 0.116 mmol) in THF (3 mL) using lithium hydroxide (22.81 mg, 0.953 mmol) in water (1 mL) to afford after work up and purification using method-M, (R)-2-(2-((5-(1-aminoisoquinolin-5-yl)-1′-ethyl-2,3-dihydrospiro[indene-1,4′-piperidin]-3-yl)oxy)phenyl)acetic acid (418c) (28 mg, 17% yield) HCl salt as a white solid; ¹H NMR (300 MHz, DMSO-d₆) δ 13.44 (s, 1H, D₂O exchangeable), 11.92 (s, 1H, D₂O exchangeable), 10.58 (s, 1H, D₂O) exchangeable), 9.06 (s, 2H, D₂O exchangeable), 8.61 (d, J=8.2 Hz, 1H), 7.95-7.76 (m, 2H), 7.64 (d, J=7.1 Hz, 1H), 7.59-7.50 (m, 1H), 7.47-7.40 (m, 2H), 7.32-7.26 (m, 2H), 7.21 (d, J=7.2 Hz, 1H), 7.00-6.86 (m, 2H), 6.03-5.90 (m, 1H), 3.58-3.43 (m, 4H), 3.22-3.07 (m, 3H), 3.07-2.96 (m, 1H), 2.84 (dd, J=13.9, 6.9 Hz, 1H), 2.46-2.26 (m, 2H), 2.14 (dd, J=13.7, 4.3 Hz, 1H), 1.84 (t, J=15.6 Hz, 2H), 1.30 (t, J=7.2 Hz, 3H); MS (ES+): 508.3 (M+1).

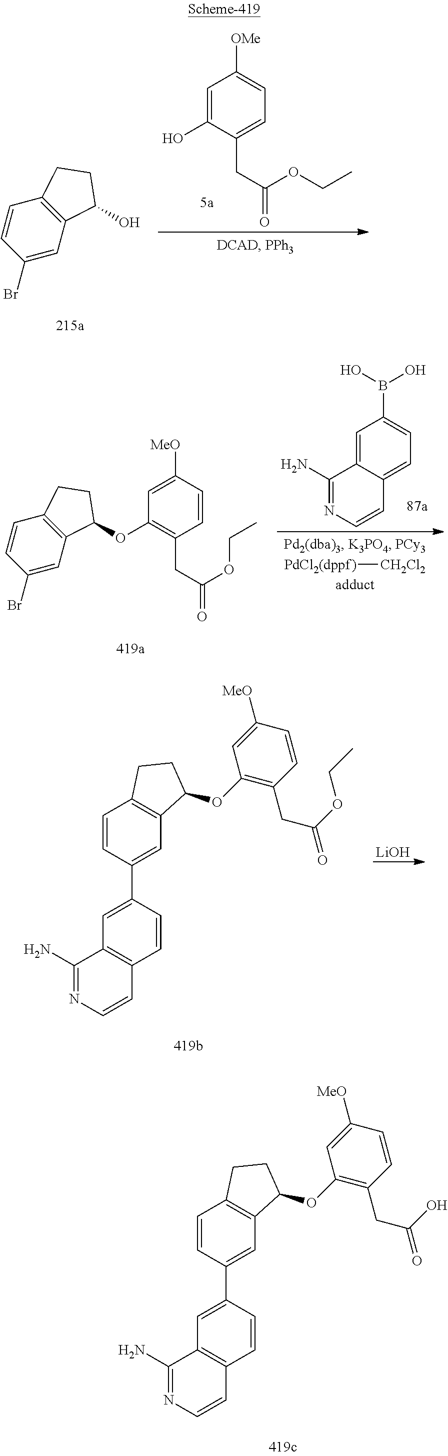

### Preparation of (R)-2-(2-((6-(1-aminoisoquinolin-7-yl)-2,3-dihydro-1H-inden-1-yl)oxy)-4-methoxyphenyl)acetic acid (419c)

### Step-1: Preparation of (R)-ethyl 2-(2-((6-bromo-2,3-dihydro-1H-inden-1-yl)oxy)-4-methoxyphenyl)acetate (419a)

Compound 419a was prepared according to the procedure reported in step-2 of scheme-2, from (S)-6-bromo-2,3-dihydro-1H-inden-1-ol (215a) (1.15 g, 5.40 mmol) in DCM (40 mL) using ethyl 2-(2-hydroxy-4-methoxyphenyl)acetate (5a) (1.362 g, 6.48 mmol). PPh₃ (1.557 g, 5.94 mmol) and a solution of DCAD (2.180 g, 5.94 mmol) in DCM (8 mL) to afford after workup and purification using method-BR, (R)-ethyl 2-(2-((6-bromo-2,3-dihydro-1H-inden-1-yl)oxy)-4-methoxyphenyl)acetate (419a) (843 mg, 2.080 mmol, 38.5% yield) as a white solid; MS (ES+): 405.1 (M+Na); (ES−): 403.1 (M−1).

### Step-2: Preparation of (R)-ethyl 2-(2-((6-(1-aminoisoquinolin-7-yl)-2,3-dihydro-1H-inden-1-yl)oxy)-4-methoxyphenyl)acetate (419b)

Compound 419b was prepared according to the procedure reported in step-5 of scheme-1 from (R)-ethyl 2-(2-((6-bromo-2,3-dihydro-1H-inden-1-yl)oxy)-4-methoxyphenyl)acetate (419a) (200 mg, 0.493 mmol) in dioxane/Me-THF (10 mL, ratio 9:1) using (1-aminoisoquinolin-7-yl)boronic acid (87a) (121 mg, 0.642 mmol), K₃PO₄ (4M aqueous solution, 493 μl, 1.974 mmol), PCy₃ (27.7 mg, 0.099 mmol). PdCl₂(dppf)-CH₂Cl₂Adduct (40.3 mg, 0.049 mmol), Pd₂(dba)₃ (45.2 mg, 0.049 mmol) to afford after workup and purification using method-AE. (R)-ethyl 2-(2-((6-(1-aminoisoquinolin-7-yl)-2,3-dihydro-1H-inden-1-yl)oxy)-4-methoxyphenyl)acetate (419b) (110 mg, 48% yield) as a brown gel; MS (ES+): 469.2 (M+1); (ES−): 467.2 (M−1).

### Step-3: Preparation of (R)-2-(2-((6-(1-aminoisoquinolin-7-yl)-2,3-dihydro-1H-inden-1-yl)oxy)-4-methoxyphenyl)acetic acid (419c)

Compound 419c was prepared according to the procedure reported in step-2 of scheme-1, from (R)-ethyl 2-(2-((6-(1-aminoisoquinolin-7-yl)-2,3-dihydro-1H-inden-1-yl)oxy)-4-methoxyphenyl)acetate (419b) (110 mg, 0.235 mmol) in THF (5 mL) using lithium hydroxide hydrate (29.6 mg, 0.704 mmol) in water (1 mL) to afford after work up and purification using method-M. (R)-2-(2-((6-(1-aminoisoquinolin-7-yl)-2,3-dihydro-1H-inden-1-yl)oxy)-4-methoxyphenyl)acetic acid (419c) (48.7 mg, 47% yield) HCl salt as a white solid; ¹H NMR (300 MHz, DMSO-d₆) δ 13.31 (s, 1H, D₂O Exchangeable), 12.03 (s, 1H, D₂O Exchangeable), 9.14 (s, 2H, D₂O Exchangeable), 8.93-8.87 (m, 1H), 8.31 (dd. J=8.5, 1.6 Hz, 1H), 8.04 (d, J=8.5 Hz, 1H), 7.91-7.82 (m, 2H), 7.69 (d, J=6.9 Hz, 1H), 7.51 (d, J=8.4 Hz, 1H), 7.27 (d, J=6.9 Hz, 1H), 7.11 (d, J=8.3 Hz, 1H), 6.81 (d, J=2.4 Hz, 1H), 6.51 (dd, J=8.3, 2.4 Hz, 1H), 5.92 (t, J=5.7 Hz, 1H), 3.78 (s, 3H), 3.39 (s, 2H), 3.18-3.04 (m, 1H), 3.02-2.87 (m, 1H), 2.77-2.61 (m, 1H), 2.10-1.94 (m, 1H); MS (ES+): 441.2 (M+1); (ES−): 439.1 (M−1); Optical Rotation: −86.545 [MeOH, 0.275].

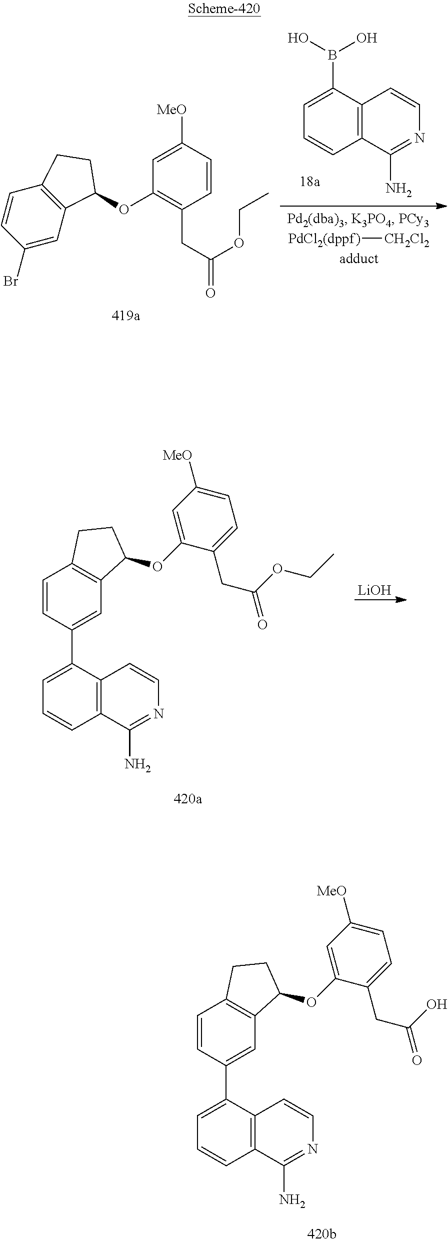

### Preparation of (R)-2-(2-((6-(1-aminoisoquinolin-5-yl)-2,3-dihydro-1H-inden-1-yl)oxy)-4-methoxyphenyl)acetic acid (420b)

### Step-1: Preparation of (R)-ethyl 2-(2-((6-(1-aminoisoquinolin-5-yl)-2,3-dihydro-1H-inden-1-yl)oxy)-4-methoxyphenyl)acetate (420a)

Compound 420a was prepared according to the procedure reported in step-5 of scheme-1 from (R)-ethyl 2-(2-((6-bromo-2,3-dihydro-1H-inden-1-yl)oxy)-4-methoxyphenyl)acetate (419a) (200 mg, 0.493 mmol) in dioxane/Me-THF (10 mL, ratio 9:1) using (1-aminoisoquinolin-5-yl)boronic acid (18a) (121 mg, 0.642 mmol), K₃PO₄ (2 M aqueous solution, (419 mg, 1.974 mmol), PCy₃ (27.7 mg, 0.099 mmol), PdCl₂(dppf)-CH₂Cl₂Adduct (40.3 mg, 0.049 mmol), Pd₂(dba)₃ (45.2 mg, 0.049 mmol) to afford after workup and purification using method-AE, (R)-ethyl 2-(2-((6-(1-aminoisoquinolin-5-yl)-2,3-dihydro-1H-inden-1-yl)oxy)-4-methoxyphenyl)acetate (420a) (97.3 mg, 42% yield) as a brown gel; MS (ES+): 469.2 (M+1); (ES−): 467.2 (M−1).

### Step-2: Preparation of (R)-2-(2-((6-(1-aminoisoquinolin-5-yl)-2,3-dihydro-1H-inden-1-yl)oxy)-4-methoxyphenyl)acetic acid (420b)

Compound 420b was prepared according to the procedure reported in step-2 of scheme-1, from (R)-ethyl 2-(2-((6-(1-aminoisoquinolin-5-yl)-2,3-dihydro-1H-inden-1-yl)oxy)-4-methoxyphenyl)acetate (420a) (97.3 mg, 0.208 mmol) in THF (5 mL) using lithium hydroxide hydrate (26.1 mg, 0.623 mmol) in water (1 mL) to afford after work up and purification using method-M, (R)-2-(2-((6-(1-aminoisoquinolin-5-yl)-2,3-dihydro-1H-inden-1-yl)oxy)-4-methoxyphenyl)acetic acid (420b) (31.2 mg, 34% yield) HCl salt as a white solid; ¹H NMR (300 MHz, DMSO-d₆) δ 13.14 (s, 1H, D₂O exchangeable), 11.91 (s, 1H, D₂O exchangeable), 9.05 (s, 2H, D₂O exchangeable), 8.58 (d, J=8.0 Hz, 1H), 7.92-7.78 (m, 2H), 7.60 (d, J=7.2 Hz, 1H), 7.50 (d, J=8.2 Hz, 1H), 7.41-7.34 (m, 2H), 7.08 (d, J=8.3 Hz, 1H), 6.94 (d, J=7.2 Hz, 1H), 6.78 (d, J=2.4 Hz, 1H), 6.49 (dd, J=8.3, 2.4 Hz, 1H), 5.91 (dd, J=6.6, 5.1 Hz, 1H), 3.75 (s, 3H), 3.35 (s, 2H), 3.22-3.07 (m, 1H), 3.04-2.91 (m, 1H), 2.79-2.64 (m, 1H), 2.14-1.99 (m, 1H); MS (ES+): 441.2 (M+1); (ES−): 439.1 (M−1); Optical Rotation: −80.0 [MeOH, 0.055].

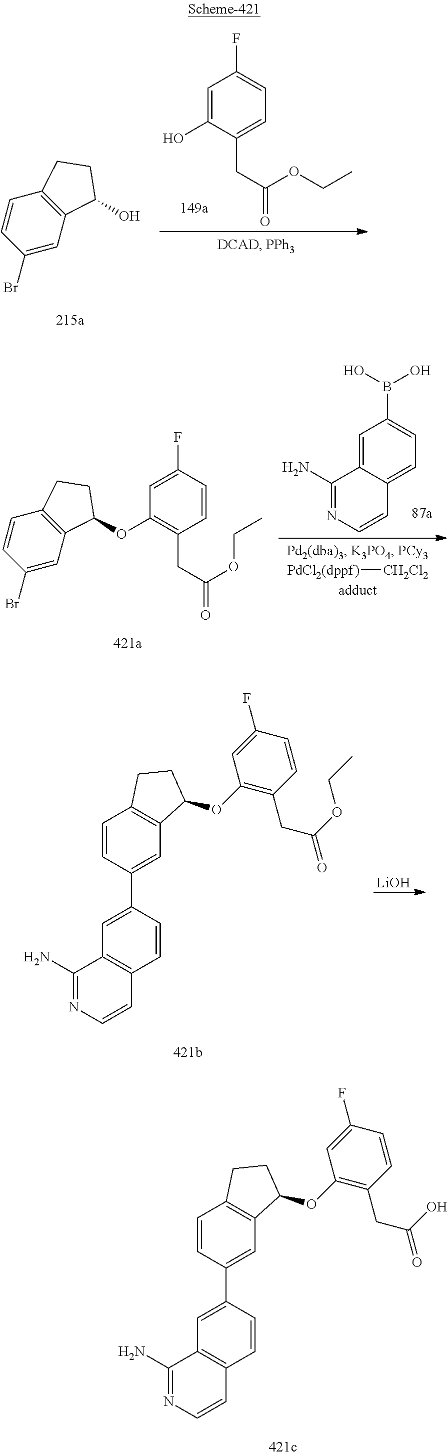

### Preparation of (R)-2-(2-((6-(1-aminoisoquinolin-7-yl)-2,3-dihydro-1H-inden-1-yl)oxy)-4-fluorophenyl)acetic acid (421c)

### Step-1: Preparation of (R)-ethyl 2-(2-((6-bromo-2,3-dihydro-1H-inden-1-yl)oxy)-4-fluorophenyl)acetate (421a)

Compound 421a was prepared according to the procedure reported in step-2 of scheme-2, from (S)-6-bromo-2,3-dihydro-1H-inden-1-ol (215a) (1.2 g, 5.63 mmol) in DCM (40 mL) using ethyl 2-(4-fluoro-2-hydroxyphenyl)acetate (149a) (1.116 g, 5.63 mmol), PPh₃ (1.625 g, 6.20 mmol) and a solution of DCAD (2.275 g, 6.20 mmol) in DCM (8 mL) to afford after workup and purification using method-BX, (R)-ethyl 2-(2-((6-bromo-2,3-dihydro-1H-inden-1-yl)oxy)-4-fluorophenyl)acetate (421a) (1.217 g, 55% yield) as a pale yellow solid; MS (ES+): 393.1 (M+1); (ES−): 391.1 (M−1).

### Step-2: Preparation of (R)-ethyl 2-(2-((6-(1-aminoisoquinolin-7-yl)-2,3-dihydro-1H-inden-1-yl)oxy)-4-fluorophenyl)acetate (421b)

Compound 421b was prepared according to the procedure reported in step-5 of scheme-1 from (R)-ethyl 2-(2-((6-bromo-2,3-dihydro-1H-inden-1-yl)oxy)-4-fluorophenyl)acetate (421a) (300 mg, 0.763 mmol) in dioxane/Me-THF (10 mL, ratio 9:1) using (1-aminoisoquinolin-7-yl)boronic acid (87a) (186 mg, 0.992 mmol), K₃PO₄ (4M aqueous solution, 763 μl, 3.05 mmol), PCy₃ (42.8 mg, 0.153 mmol). PdCl₂(dppf)-CH₂Cl₂Adduct (62.3 mg, 0.076 mmol), Pd₂(dba)₃ (69.9 mg, 0.076 mmol) to afford after workup and purification using method-AE, (R)-ethyl 2-(2-((6-(1-aminoisoquinolin-7-yl)-2,3-dihydro-1H-inden-1-yl)oxy)-4-fluorophenyl)acetate (421b) (111 mg, 32% yield) as a brown gel; MS (ES+): 457.2 (M+1); (ES−): 455.3 (M−1).

### Step-3: Preparation of (R)-2-(2-((6-(1-aminoisoquinolin-7-yl)-2,3-dihydro-1H-inden-1-yl)oxy)-4-fluorophenyl)acetic acid (421c)

Compound 421c was prepared according to the procedure reported in step-2 of scheme-1, from (R)-ethyl 2-(2-((6-(1-aminoisoquinolin-7-yl)-2,3-dihydro-1H-inden-1-yl)oxy)-4-fluorophenyl)acetate (421b) (111 mg, 0.243 mmol) in THF (5 mL) using lithium hydroxide hydrate (30.6 mg, 0.729 mmol) in Water (1 mL), to afford after work up and purification using method-M, (R)-2-(2-((6-(1-aminoisoquinolin-7-yl)-2,3-dihydro-1H-inden-1-yl)oxy)-4-fluorophenyl)acetic acid (421c) (27.1 mg, 26% yield) HCl salt as a white solid; ¹H NMR (300 MHz, DMSO-d₆) δ 13.19 (s, 1H, D₂O Exchangeable), 12.10 (s, 1H, D₂O Exchangeable), 9.09 (s, 2H, D₂O Exchangeable), 8.89 (s, 1H), 8.31 (dd, J=8.5, 1.7 Hz, 1H), 8.04 (d, J=8.5 Hz, 1H), 7.91-7.84 (m, 2H), 7.69 (d, J=6.9 Hz, 1H), 7.52 (d, J=8.5 Hz, 1H), 7.29-7.22 (m, 2H), 7.22-7.14 (m, 1H), 6.75 (td, J=8.5, 2.5 Hz, 1H), 5.96 (t, 1H), 3.45 (s, 2H), 3.19-3.03 (m, 1H), 3.03-2.87 (m, 1H), 2.79-2.61 (m, 1H), 2.09-1.95 (m, 1H); ¹⁹F NMR (282 MHz, DMSO-d₆) δ −112.86; MS (ES+): 429.2 (M+1); (ES−): 427.1 (M−1); Optical Rotation: −154.29 [MeOH, 0.07].

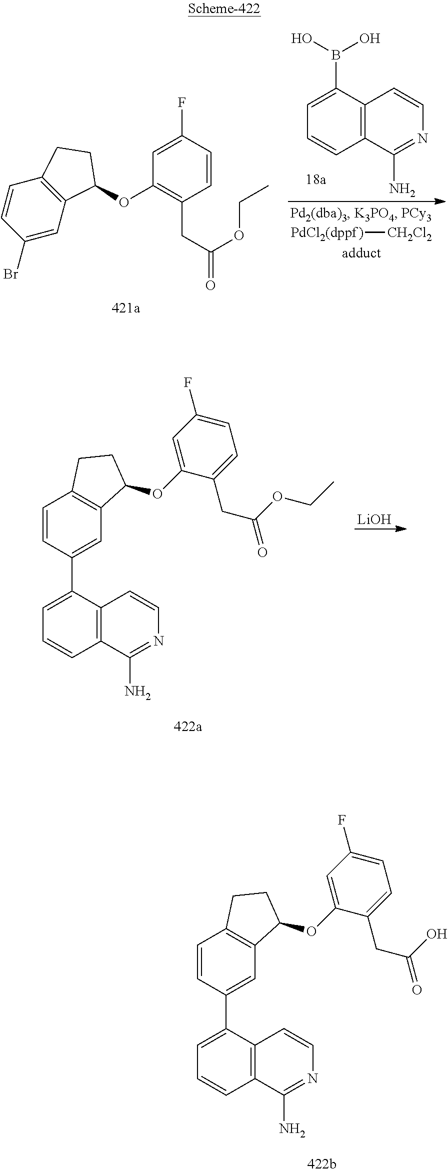

### Preparation of (R)-2-(2-((6-(1-aminoisoquinolin-5-yl)-2,3-dihydro-1H-inden-1-yl)oxy)-4-fluorophenyl)acetic acid (422b)

### Step-1: Preparation of (R)-ethyl 2-(2-((6-(1-aminoisoquinolin-5-yl)-2,3-dihydro-1H-inden-1-yl)oxy)-4-fluorophenyl)acetate (422a)

Compound 422a was prepared according to the procedure reported in step-5 of scheme-1 from (R)-ethyl 2-(2-((6-bromo-2,3-dihydro-1H-inden-1-yl)oxy)-4-fluorophenyl)acetate (421a) (300 mg, 0.763 mmol) in dioxane/Me-THF (10 mL, ratio 9:1) using (1-aminoisoquinolin-5-yl)boronic acid (18a) (186 mg, 0.992 mmol), K₃PO₄ (2 M aqueous solution, 648 mg, 3.05 mmol), PCy₃ (42.8 mg, 0.153 mmol), PdCl₂(dppf)-CH₂Cl₂Adduct (62.3 mg, 0.076 mmol), Pd₂(dba)₃ (69.9 mg, 0.076 mmol) to afford after workup and purification using method-AE, (R)-ethyl 2-(2-((6-(1-aminoisoquinolin-5-yl)-2,3-dihydro-1H-inden-1-yl)oxy)-4-fluorophenyl)acetate (422a) (127 mg, 37% yield); MS (ES+): 457.2 (M+1); (ES−): 455.2 (M−1).

### Step-2: Preparation of (R)-2-(2-((6-(1-aminoisoquinolin-5-yl)-2,3-dihydro-1H-inden-1-yl)oxy)-4-fluorophenyl)acetic acid (422b)

Compound 422b was prepared according to the procedure reported in step-2 of scheme-1, from (R)-ethyl 2-(2-((6-(1-aminoisoquinolin-5-yl)-2,3-dihydro-1H-inden-1-yl)oxy)-4-fluorophenyl)acetate (422a) (127 mg, 0.278 mmol) in THF (5 mL) using lithium hydroxide hydrate (35.0 mg, 0.835 mmol) in water (1 mL) to afford after work up and purification using method-M, (R)-2-(2-((6-(1-aminoisoquinolin-5-yl)-2,3-dihydro-1H-inden-1-yl)oxy)-4-fluorophenyl)acetic acid (422b) (42.4 mg, 36% yield) HCl salt as a white solid; ¹H NMR (300 MHz, DMSO-d₆) δ 13.19 (s, 1H, D₂O Exchangeable), 12.00 (s, 1H, D₂O Exchangeable), 9.11 (s, 2H, D₂O Exchangeable), 8.60 (d, J=8.0 Hz, 1H), 7.93-7.78 (m, 2H), 7.60 (d, J=7.3 Hz, 1H), 7.51 (d, J=8.4 Hz, 1H), 7.43-7.35 (m, 2H), 7.27-7.08 (m, 2H), 6.95 (d, J=7.2 Hz, 1H), 6.73 (td, J=8.4, 2.4 Hz, 1H), 5.94 (t, J=5.8 Hz, 1H), 3.42 (d, J=2.7 Hz, 2H), 3.20-3.08 (m, 1H), 3.06-2.90 (m, 1H), 2.80-2.68 (m, 1H), 2.10-1.97 (m, 1H); ¹⁹F NMR (282 MHz, DMSO) δ −112.88; MS (ES+): 429.1 (M+1); (ES−): 427.1 (M−1); Optical Rotation: −65.263 [MeOH, 0.095].

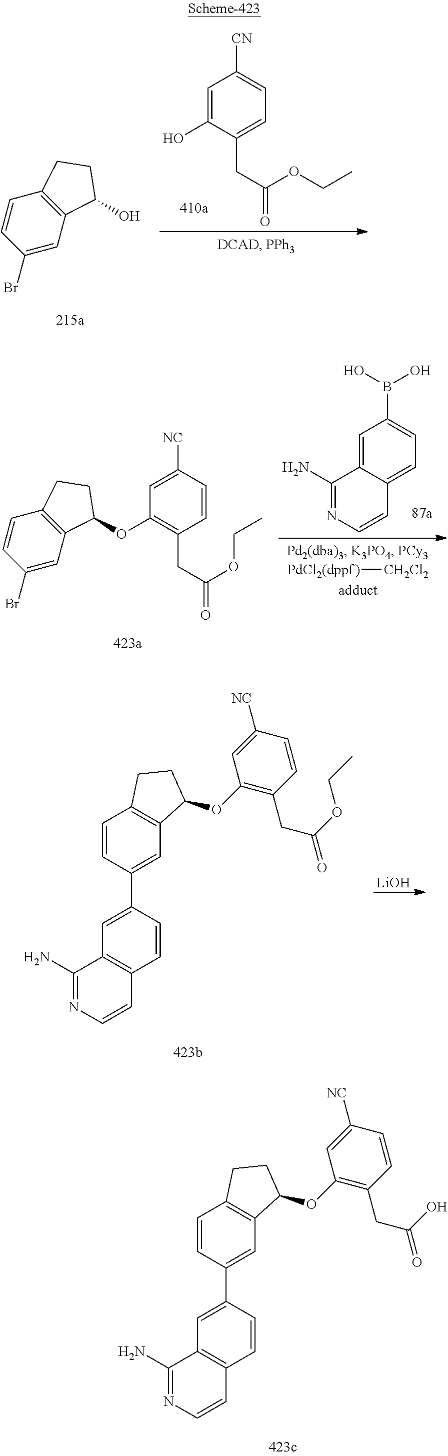

### Preparation of (R)-2-(2-((6-(1-aminoisoquinolin-7-yl)-2,3-dihydro-1H-inden-1-yl)oxy)-4-cyanophenyl)acetic acid (423c)

### Step-1: Preparation of (R)-ethyl 2-(2-((6-bromo-2,3-dihydro-1H-inden-1-yl)oxy)-4-cyanophenyl)acetate (423a)

Compound 423a was prepared according to the procedure reported in step-2 of scheme-2, from (S)-6-bromo-2,3-dihydro-1H-inden-1-ol (215a) (1.2 g, 5.63 mmol) in DCM (40 mL) using ethyl 2-(4-cyano-2-hydroxyphenyl)acetate (410a) (1.156 g, 5.63 mmol), PPh₃ (1.625 g, 6.20 mmol) and a solution of DCAD (2.275 g, 6.20 mmol) in DCM (8 mL) to afford after workup and purification using method-BX, (R)-ethyl 2-(2-((6-bromo-2,3-dihydro-1H-inden-1-yl)oxy)-4-cyanophenyl)acetate (423a) (1.315 g, 58% yield) as a white solid; MS (ES+): 422.1 (M+Na).

### Step-2: Preparation of (R)-ethyl 2-(2-((6-(1-aminoisoquinolin-7-yl)-2,3-dihydro-1H-inden-1-yl)oxy)-4-cyanophenyl)acetate (423b)

Compound 423b was prepared according to the procedure reported in step-5 of scheme-1 from (R)-ethyl 2-(2-((6-bromo-2,3-dihydro-1H-inden-1-yl)oxy)-4-cyanophenyl)acetate (423a) (300 mg, 0.749 mmol) in dioxane/Me-THF (10 mL, ratio 9:1) using (1-aminoisoquinolin-7-yl)boronic acid (87a) (183 mg, 0.974 mmol), K₃PO₄ (4M aqueous solution, 749 μl, 3.00 mmol), PCy₃ (42.0 mg, 0.150 mmol), PdCl₂(dppf)-CH₂Cl₂Adduct (61.2 mg, 0.075 mmol), Pd₂(dba)₃ (68.6 mg, 0.075 mmol) to afford after workup and purification using method-AE, (R)-ethyl 2-(2-((6-(1-aminoisoquinolin-7-yl)-2,3-dihydro-1H-inden-1-yl)oxy)-4-cyanophenyl)acetate (423b) (125 mg, 36% yield); MS (ES+): 464.2 (M+1); (ES−): 462.2 (M−1).

### Step-3: Preparation of (R)-2-(2-(6-(1-aminoisoquinolin-7-yl)-2,3-dihydro-1H-inden-1-yl)oxy)-4-cyanophenyl)acetic acid (423c)

Compound 423c was prepared according to the procedure reported in step-2 of scheme-1, from (R)-ethyl 2-(2-((6-(1-aminoisoquinolin-7-yl)-2,3-dihydro-1H-inden-1-yl)oxy)-4-cyanophenyl)acetate (423b) (125 mg, 0.270 mmol) in THF (5 mL) using lithium hydroxide hydrate (33.9 mg, 0.809 mmol) in water (1 mL) to afford after work up and purification using method-M, (R)-2-(2-((6-(1-aminoisoquinolin-7-yl)-2,3-dihydro-1H-inden-1-yl)oxy)-4-cyanophenyl)acetic acid (423c) (23.5 mg, 20% yield) HCl salt as a white solid; ¹H NMR (300 MHz, DMSO-d₆) δ 12.88 (s, 1H, D₂O Exchangeable), 9.10 (s, 2H, D₂O Exchangeable), 8.90 (s, 1H), 8.31 (dd, J=8.5, 1.6 Hz, 1H), 8.03 (d, J=8.5 Hz, 1H), 7.91-7.85 (m, 2H), 7.75 (s, 1H), 7.70 (d, J=6.8 Hz, 1H), 7.57-7.49 (m, 1H), 7.48-7.38 (m, 2H), 7.26 (d, J=6.8 Hz, 1H), 6.09-6.00 (m, 1H), 3.57 (s, 2H), 3.19-3.04 (m, 1H), 3.03-2.89 (m, 1H), 2.83-2.68 (m, 1H), 2.08-1.94 (m, 1H). MS (ES+): 436.2 (M+1); Optical Rotation: −129.23 [MeOH, 0.06]; FT-1R: 2230.8 cm⁻¹.

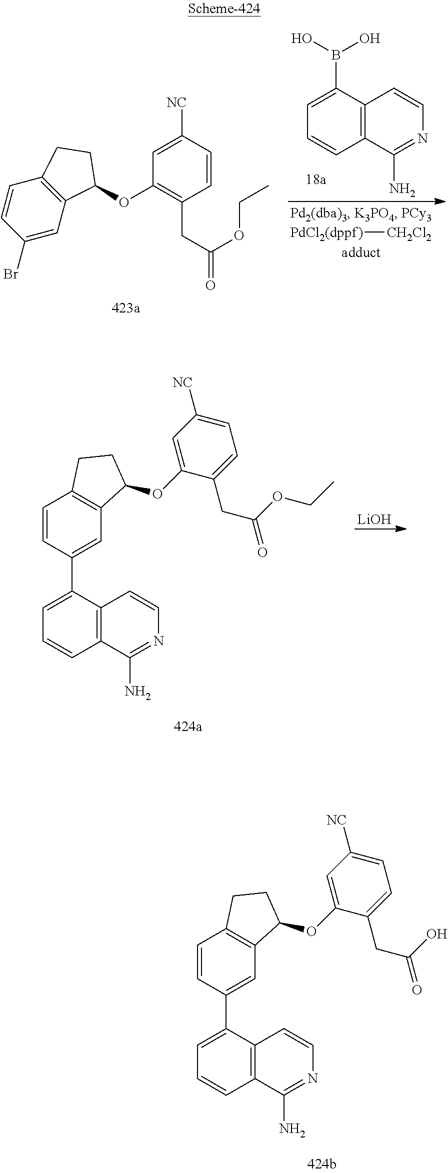

### Preparation of (R)-2-(2-((6-(1-aminoisoquinolin-5-yl)-2,3-dihydro-1H-inden-1-yl)oxy)-4-cyanophenyl)acetic acid (424b)

### Step-1: Preparation of (R)-ethyl 2-(2-((6-(1-aminoisoquinolin-5-yl)-2,3-dihydro-1H-inden-1-yl)oxy)-4-cyanophenyl)acetate (424a)

Compound 424a was prepared according to the procedure reported in step-5 of scheme-1 from (R)-ethyl 2-(2-((6-bromo-2,3-dihydro-1H-inden-1-yl)oxy)-4-cyanophenyl)acetate (423a) (300 mg, 0.749 mmol) in dioxane/Me-THF (10 mL, ratio 9:1) using (1-aminoisoquinolin-5-yl)boronic acid (18a) (183 mg, 0.974 mmol). K₃PO₄ (2 M aqueous solution, 636 mg, 3.00 mmol), PCy₃ (42.0 mg, 0.150 mmol), PdCl₂(dppf)-CH₂Cl₂Adduct (61.2 mg, 0.075 mmol) and Pd₂(dba)₃ (68.6 mg, 0.075 mmol) to afford after workup and purification using method-AE, (R)-ethyl 2-(2-((6-(1-aminoisoquinolin-5-yl)-2,3-dihydro-1H-inden-1-yl)oxy)-4-cyanophenyl)acetate (424a) (116 mg, 33% yield); MS (ES+): 464.2 (M+1); (ES−): 462.3 (M−1).

### Step-2: Preparation of (R)-2-(2-((6-(1-aminoisoquinolin-5-yl)-2,3-dihydro-1H-inden-1-yl)oxy)-4-cyanophenyl)acetic acid (424b)

Compound 424b was prepared according to the procedure reported in step-2 of scheme-1, from (R)-ethyl 2-(2-((6-(1-aminoisoquinolin-5-yl)-2,3-dihydro-1H-inden-1-yl)oxy)-4-cyanophenyl)acetate (424a) (116 mg, 0.250 mmol) in THF (5 mL) using lithium hydroxide hydrate (31.5 mg, 0.751 mmol) in water (1 mL) to afford after work up and purification using method-M, (R)-2-(2-((6-(1-aminoisoquinolin-5-yl)-2,3-dihydro-1H-inden-1-yl)oxy)-4-cyanophenyl)acetic acid (424b) (34.3 mg, 32% yield) HCl salt as a white solid; ¹H NMR (300 MHz, DMSO-d₆) δ 13.22 (s, 1H, D₂O Exchangeable), 12.17 (s, 1H, D₂O Exchangeable), 9.12 (s, 2H, D₂O Exchangeable), 8.60 (d, J=8.0 Hz, 1H), 7.93-7.80 (m, 2H), 7.72 (s, 1H), 7.61 (d, J=7.3 Hz, 1H), 7.52 (d, J=7.9 Hz, 1H), 7.45-7.35 (m, 4H), 6.95 (d, J=7.3 Hz, 1H), 6.02 (t, J=5.7 Hz, 1H), 3.55 (d, J=2.3 Hz, 2H), 3.22-3.08 (m, 1H), 3.05-2.91 (m, 1H), 2.87-2.70 (m, 1H), 2.10-1.97 (m, 1H); MS (ES+): 436.2 (M+1); Optical Rotation: − 40.0 [MeOH, 0.105]; FT-1R: 2227.1 cm⁻¹.

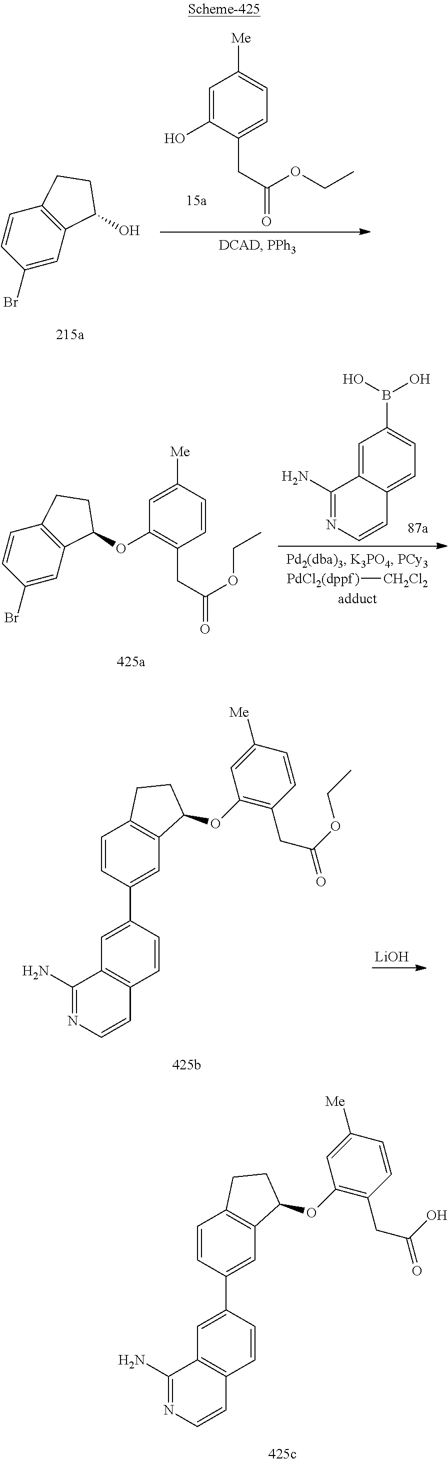

### Preparation of (R)-2-(2-((6-(1-aminoisoquinolin-7-yl)-2,3-dihydro-1H-inden-1-yl)oxy)-4-methylphenyl)acetic acid (425c)

### Step-1: Preparation of (R)-ethyl 2-(2-((6-bromo-2,3-dihydro-H-inden-1-yl)oxy)-4-methylphenyl)acetate (425a)

Compound 425a was prepared according to the procedure reported in step-2 of scheme-2, from (S)-6-bromo-2,3-dihydro-1H-inden-1-ol (215a) (1.2 g, 5.63 mmol) in DCM (40 mL) ethyl 2-(2-hydroxy-4-methylphenyl)acetate (15a) (1.094 g, 5.63 mmol), PPh₃ (1.625 g, 6.20 mmol) and a solution of DCAD (2.275 g, 6.20 mmol) in DCM (8 mL) to afford after workup and purification using method-BR. (R)-ethyl 2-(2-((6-bromo-2,3-dihydro-1H-inden-1-yl)oxy)-4-methylphenyl)acetate (425a) (1.231 g, 56% yield) as a white solid; MS (ES+): 389.1 (M+1); (ES−): 387.1 (M−1).

### Step-2: Preparation of (R)-ethyl 2-(2-((6-(1-aminoisoquinolin-7-yl)-2,3-dihydro-1H-inden-1-yl)oxy)-4-methylphenyl)acetate (425b)

Compound 425b was prepared according to the procedure reported in step-5 of scheme-1, from (R)-ethyl 2-(2-((6-bromo-2,3-dihydro-1H-inden-1-yl)oxy)-4-methylphenyl)acetate (425a) (300 mg, 0.771 mmol) in dioxane/Me-THF (10 mL, ratio 9:1) using (1-aminoisoquinolin-7-yl)boronic acid (87a) (188 mg, 1.002 mmol), K₃PO₄ (4M aqueous solution, 771 μl, 3.08 mmol), PCy₃ (43.2 mg, 0.154 mmol), PdCl₂(dppf)-CH₂Cl₂Adduct (62.9 mg, 0.077 mmol) and Pd₂(dba)₃ (70.6 mg, 0.077 mmol) to afford after workup and purification using method-AE, (R)-ethyl 2-(2-((6-(1-aminoisoquinolin-7-yl)-2,3-dihydro-1H-inden-1-yl)oxy)-4-methylphenyl)acetate (425b) (127 mg, 36% yield); MS (ES+): 453.2 (M+1); (ES−): 451.3 (M−1).

### Step-3: Preparation of (R)-2-(2-((6-(1-aminoisoquinolin-7-yl)-2,3-dihydro-1H-inden-1-yl)oxy)-4-methylphenyl)acetic acid (425c)

Compound 425c was prepared according to the procedure reported in step-2 of scheme-1, from (R)-ethyl 2-(2-((6-(1-aminoisoquinolin-7-yl)-2,3-dihydro-1H-inden-1-yl)oxy)-4-methylphenyl)acetate (425b) (127 mg, 0.281 mmol) in THF (5 mL) using lithium hydroxide hydrate (35.3 mg, 0.842 mmol) in water (1 mL) to afford after work up and purification using method-M, (R)-2-(2-((6-(1-aminoisoquinolin-7-yl)-2,3-dihydro-1H-inden-1-yl)oxy)-4-methylphenyl)acetic acid (425c) (32.3 mg, 27% yield) HCl salt as a white solid; ¹H NMR (300 MHz, DMSO-d₆) δ 13.12 (s, 1H, D₂O exchangeable), 12.06 (s, 1H, D₂O exchangeable), 9.08 (s, 2H, D₂O Exchangeable), 8.89 (s, 1H), 8.30 (dd, J=8.4, 1.6 Hz, 1H), 8.03 (d, J=8.5 Hz, 1H), 7.91-7.82 (m, 2H), 7.69 (d, J=6.9 Hz, 1H), 7.51 (d, J=8.2 Hz, 1H), 7.26 (d, J=6.9 Hz, 1H), 7.11-7.04 (m, 2H), 6.74 (d, J=7.6 Hz, 1H), 5.89 (t, J=5.7 Hz, 1H), 3.42 (s, 2H), 3.18-3.05 (m, 1H), 3.01-2.89 (m, 1H), 2.76-2.63 (m, 1H), 2.34 (s, 3H), 2.09-1.96 (m, 1H); MS (ES+): 425.2 (M+1); (ES−): 423.1 (M−1); Optical Rotation: −137.85 [MeOH, 0.13].

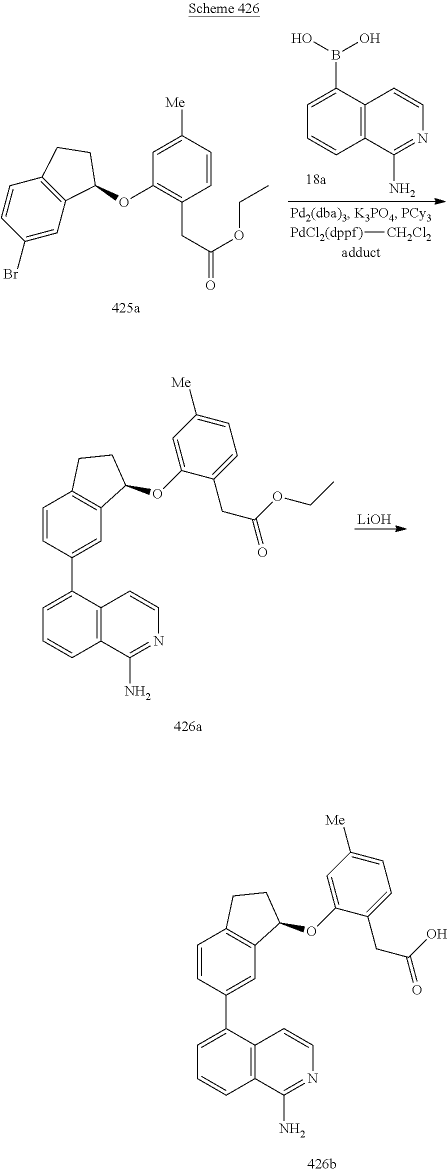

### Preparation of (R)-2-(2-((6-(1-aminoisoquinolin-5-yl)-2,3-dihydro-1H-inden-1-yl)oxy)-4-methylphenyl)acetic acid (426b)

### Step-1: Preparation of (R)-ethyl 2-(2-((6-(1-aminoisoquinolin-5-yl)-2,3-dihydro-1H-inden-1-yl)oxy)-4-methylphenyl)acetate (426a)

Compound 426a was prepared according to the procedure reported in step-5 of scheme 1, from (R)-ethyl 2-(2-((6-bromo-2,3-dihydro-1H-inden-1-yl)oxy)-4-methylphenyl)acetate (425a) (300 mg, 0.771 mmol) in dioxane/Me-THF (10 mL, ratio 9:1) using (1-aminoisoquinolin-5-yl)boronic acid (18a) (188 mg, 1.002 mmol), K₃PO₄ (2 M aqueous solution, 654 mg, 3.08 mmol), PCy₃ (43.2 mg, 0.154 mmol), PdCl₂(dppf)-CH₂Cl₂ adduct (62.9 mg, 0.077 mmol) and Pd₂(dba)₃ (70.6 mg, 0.077 mmol) to afford after workup and purification using method-AE, (R)-ethyl 2-(2-((6-(1-aminoisoquinolin-5-yl)-2,3-dihydro-1H-inden-1-yl)oxy)-4-methylphenyl)acetate (426a) (117 mg, 34% yield); MS (ES+): 453.2 (M+1); (ES−): 451.2 (M−1).

### Step-2: Preparation of (R)-2-(2-((6-(1-aminoisoquinolin-5-yl)-2,3-dihydro-1H-inden-1-yl)oxy)-4-methylphenyl)acetic acid (426b)

Compound 426b was prepared according to the procedure reported in step-2 of scheme 1, from (R)-ethyl 2-(2-((6-(1-aminoisoquinolin-5-yl)-2,3-dihydro-1H-inden-1-yl)oxy)-4-methylphenyl)acetate (426a) (117 mg, 0.259 mmol) in THF (S mL) using lithium hydroxide hydrate (32.5 mg, 0.776 mmol) in water (1 mL) to afford after work up and purification using method-M, (R)-2-(2-((6-(1-aminoisoquinolin-5-yl)-2,3-dihydro-1H-inden-1-yl)oxy)-4-methylphenyl)acetic acid (426b) (28.7 mg, 26% yield) HCl salt as a white solid; ¹H NMR (300 MHz, DMSO-d₆) δ 13.16 (s, 1H, D₂O Exchangeable), 11.93 (s, 1H, D₂O Exchangeable), 9.08 (s, 2H, D₂O Exchangeable), 8.59 (d, J=7.8 Hz, 1H), 7.93-7.79 (m, 2H), 7.60 (d, J=7.3 Hz, 1H), 7.50 (d, J=8.2 Hz, 1H), 7.42-7.34 (m, 2H), 7.09-7.02 (m, 2H), 6.96 (d, J=7.2 Hz, 1H), 6.72 (d, J=7.6 Hz, 1H), 5.88 (t, J=6.0 Hz, 1H), 3.38 (d, J=3.1 Hz, 2H), 3.20-3.08 (m, 1H), 3.04-2.91 (m, 1H), 2.81-2.66 (m, 1H), 2.30 (s, 3H), 2.13-1.98 (m, 1H); MS (ES+): 425.2 (M+1); Optical Rotation: −80.0 [MeOH, 0.095].

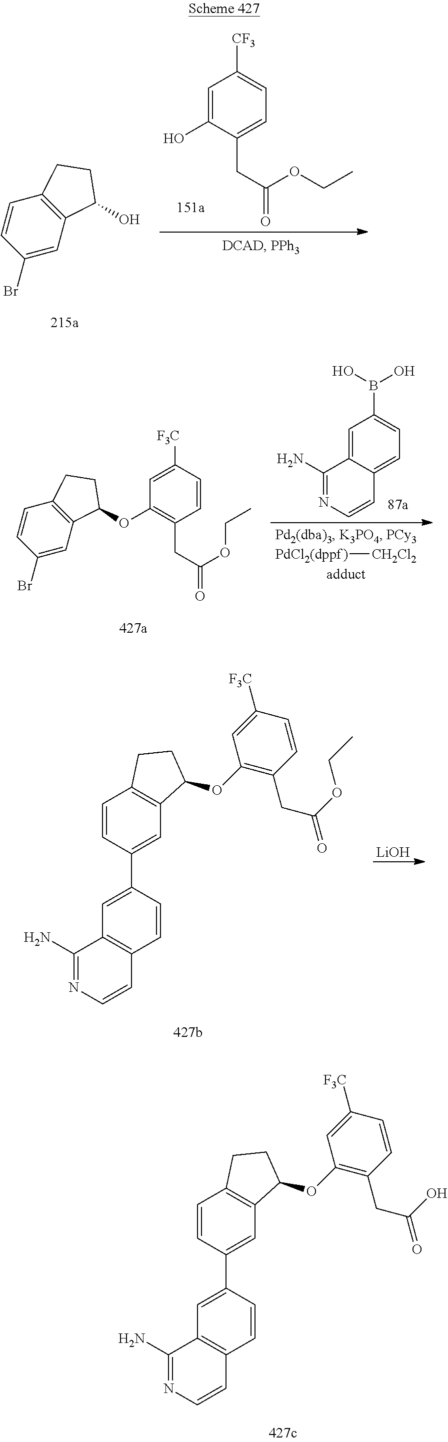

### Preparation of (R)-2-(2-((6-(1-aminoisoquinolin-7-yl)-2,3-dihydro-1H-inden-1-yl)oxy)-4-(trifluoromethyl)phenyl)acetic acid (427c)

### Step-1: Preparation of (R)-ethyl 2-(2-((6-bromo-2,3-dihydro-1H-inden-1-yl)oxy)-4-(trifluoromethyl)phenyl)acetate (427a)

Compound 427a was prepared according to the procedure reported in step-2 of scheme 2, from (S)-6-bromo-2,3-dihydro-1H-inden-1-ol (215a) (1.2 g, 5.63 mmol) in DCM (40 mL) using ethyl 2-(2-hydroxy-4-(trifluoromethyl)phenyl)acetate (151a) (1.398 g, 5.63 mmol), PPh₃ (1.625 g, 6.20 mmol) and a solution of DCAD (2.275 g, 6.20 mmol) in DCM (8 mL) to afford after workup and purification using method-BR, (R)-ethyl 2-(2-((6-bromo-2,3-dihydro-1H-inden-1-yl)oxy)-4-(trifluoromethyl)phenyl)acetate (427a) (987 mg, 40% yield) as an off-white solid; MS (ES−): 441.1 (M−1).

### Step-2: Preparation of (R)-ethyl 2-(2-((6-(1-aminoisoquinolin-7-yl)-2,3-dihydro-1H-inden-1-yl)oxy)-4-(trifluoromethyl)phenyl)acetate (427b)

Compound 427b was prepared according to the procedure reported in step-5 of scheme 1, from (R)-ethyl 2-(2-((6-bromo-2,3-dihydro-1H-inden-1-yl)oxy)-4-(trifluoromethyl)phenyl)acetate (427a) (300 mg, 0.677 mmol) in dioxane/Me-THF (10 mL, ratio 9:1) using (1-aminoisoquinolin-7-yl)boronic acid (87a) (165 mg, 0.880 mmol), K₃PO₄ (4M aqueous solution, 677 μl, 2.71 mmol), PCy₃ (38.0 mg, 0.135 mmol), PdCl₂(dppf)-CH₂Cl₂ adduct (55.3 mg, 0.068 mmol) and Pd₂(dba)₃ (62.0 mg, 0.068 mmol) to afford after workup and purification using method-AE, (R)-ethyl 2-(2-((6-(1-aminoisoquinolin-7-yl)-2,3-dihydro-1H-inden-1-yl)oxy)-4-(trifluoromethyl)phenyl)acetate (427b) (131 mg, 38% yield); MS (ES+): 507.2 (M+1); (ES−): 505.1 (M−1).

### Step-3: Preparation of (R)-2-(2-((6-(1-aminoisoquinolin-7-yl)-2,3-dihydro-1H-inden-1-yl)oxy)-4-(trifluoromethyl)phenyl)acetic acid (427c)

Compound 427c was prepared according to the procedure reported in step-2 of scheme 1, from (R)-ethyl 2-(2-((6-(1-aminoisoquinolin-7-yl)-2,3-dihydro-1H-inden-1-yl)oxy)-4-(trifluoromethyl)phenyl)acetate (427b) (131 mg, 0.259 mmol) in THF (5 mL) using lithium hydroxide hydrate (32.6 mg, 0.776 mmol) in water (1 mL) to afford after work up and purification using method-M, followed by purification method-R, (R)-2-(2-((6-(1-aminoisoquinolin-7-yl)-2,3-dihydro-1H-inden-1-yl)oxy)-4-(trifluoromethyl)phenyl)acetic acid (427c) (32.2 mg, 26% yield) as a free base; ¹H NMR (300 MHz, DMSO-d₆) δ 12.30 (s, 1H), 8.52 (s, 1H), 7.99-7.91 (m, 1H), 7.87-7.81 (m, 2H), 7.81-7.75 (m, 2H), 7.55 (s, 1H), 7.47 (dd, J=7.8, 4.2 Hz, 2H), 7.31 (d, J=7.8 Hz, 1H), 7.03-6.86 (m, 3H), 6.08 (t, J=5.5 Hz, 1H), 3.58 (s, 2H), 3.19-3.05 (m, 1H), 3.03-2.90 (m, 1H), 2.75-2.70 (m, 1H), 2.09-1.96 (m, 1H); 1ºF NMR (282 MHz, DMSO-d₆) δ −60.64; MS (ES+): 479.1 (M+1); (ES−): 477.1 (M−1); Optical Rotation: −145.714 [MeOH, 0.07].

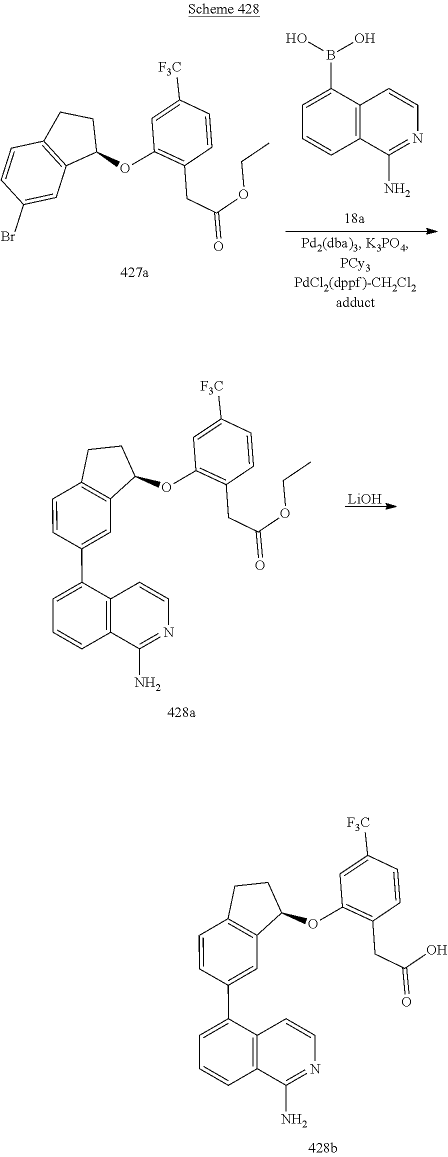

### Preparation of (R)-2-(2-((6-(1-aminoisoquinolin-5-yl)-2,3-dihydro-1H-inden-1-yl)oxy)-4-(trifluoromethyl)phenyl)acetic acid (428b)

### Step-1: Preparation of (R)-ethyl 2-(2-((6-(1-aminoisoquinolin-5-yl)-2,3-dihydro-1H-inden-1-yl)oxy)-4-(trifluoromethyl)phenyl)acetate (428a)

Compound 428a was prepared according to the procedure reported in step-5 of scheme 1, from (R)-ethyl 2-(2-((6-bromo-2,3-dihydro-1H-inden-1-yl)oxy)-4-(trifluoromethyl)phenyl)acetate (427a) (300 mg, 0.677 mmol) in dioxane/Me-THF (10 mL, ratio 9:1) using (I-aminoisoquinolin-5-yl)boronic acid (18a) (165 mg, 0.880 mmol), K₃PO₄ (2 M aqueous solution, 575 mg, 2.71 mmol), PCy₃ (38.0 mg, 0.135 mmol), PdCl₂(dppf)-CH₂Cl₂ adduct (55.3 mg, 0.068 mmol) and Pd₂(dba)₃ (62.0 mg, 0.068 mmol) to afford after workup and purification using method-AE. (R)-ethyl 2-(2-((6-(1-aminoisoquinolin-5-yl)-2,3-dihydro-1H-inden-1-yl)oxy)-4-(trifluoromethyl)phenyl)acetate (428a) (133 mg, 39% yield); MS (ES+): 507.2 (M+1); (ES−): 505.1 (M−1).

### Step-2: Preparation of (R)-2-(2-((6-(1-aminoisoquinolin-5-yl)-2,3-dihydro-1H-inden-1-yl)oxy)-4-(trifluoromethyl)phenyl)acetic acid (428b)

Compound 428b was prepared according to the procedure reported in step-2 of scheme 1, from (R)-ethyl 2-(2-((6-(1-aminoisoquinolin-5-yl)-2,3-dihydro-1H-inden-1-yl)oxy)-4-(trifluoromethyl)phenyl)acetate (428a) (133 mg, 0.263 mmol) in THF (5 ml) using lithium hydroxide hydrate (33.1 mg, 0.788 mmol) in water (1 mL) to afford after work up and purification using method-M, (R)-2-(2-((6-(1-aminoisoquinolin-5-yl)-2,3-dihydro-1H-inden-1-yl)oxy)-4-(trifluoromethyl)phenyl)acetic acid (428b)(41.3 mg, 0.086 mmol, 32.9% yield) HCl salt as a white solid; ¹H NMR (300 MHz, DMSO-d₆) δ 13.21 (s, 1H, D₂O Exchangeable), 12.18 (s, 1H, D₂O Exchangeable), 9.03 (s, 2H, D₂O Exchangeable), 8.59 (d, J=7.9 Hz, 1H), 7.90-7.84 (m, 1H), 7.84-7.78 (m, 1H), 7.61 (d, J=7.2 Hz, 1H), 7.56-7.48 (m, 2H), 7.47-7.36 (m, 3H), 7.28 (d, J=7.8 Hz, 1H), 6.94 (d, J=7.2 Hz, 1H), 6.08 (t, J=5.8 Hz, 1H), 3.55 (d, J=2.5 Hz, 2H), 3.21-3.09 (m, 1H), 3.06-2.93 (m, 1H), 2.82-2.67 (m, 1H), 2.13-1.99 (m, 1H); ¹⁹F NMR (282 MHz, DMSO-d₆) δ −60.70; MS (ES+): 479.2 (M+1); Optical Rotation: −60.80 [MeOH, 0.125].

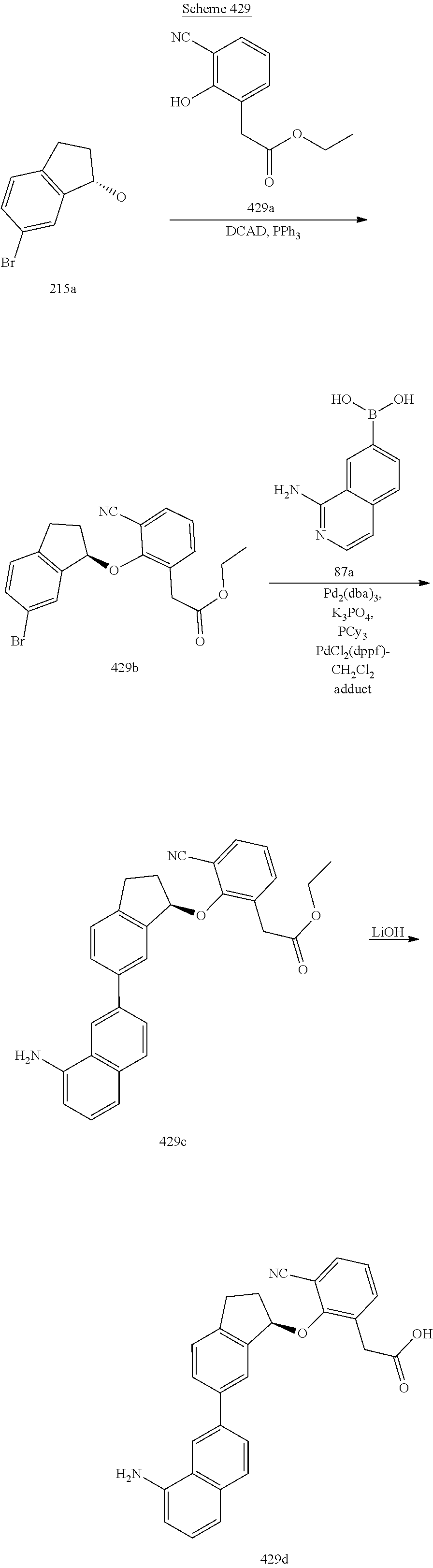

### Preparation of (R)-2-(2-((6-(1-aminoisoquinolin-7-yl)-2,3-dihydro-1H-inden-1-yl)oxy)-3-cyanophenyl)acetic acid (429d)

### Step-1: Preparation of (R)-ethyl 2-(2-((6-bromo-2,3-dihydro-1H-inden-1-yl)oxy)-3-cyanophenyl)acetate (429b)

Compound 429b was prepared according to the procedure reported in step-2 of scheme 2, from (S)-6-bromo-2,3-dihydro-1H-inden-1-ol (215a) (1.3 g, 6.10 mmol) in DCM (40 mL) using ethyl 2-(3-cyano-2-hydroxyphenyl)acetate (429a) (1.252 g, 6.10 mmol; CAS #1261879-19-9), PPh₃ (1.760 g, 6.71 mmol) and a solution of DCAD (2.464 g, 6.71 mmol) in DCM (8 mL) to afford after workup and purification using method-BR, (R)-ethyl 2-(2-((6-bromo-2,3-dihydro-1H-inden-1-yl)oxy)-3-cyanophenyl)acetate (429b) (1.237 g, 51% yield) as a pale yellow solid; MS (ES+): 422.0 (M+Na).

### Step-2: Preparation of (R)-ethyl 2-(2-((6-(1-aminoisoquinolin-7-yl)-2,3-dihydro-1H-inden-1-yl)oxy)-3-cyanophenyl)acetate (429c)

Compound 429c was prepared according to the procedure reported in step-5 of scheme 1, from (R)-ethyl 2-(2-((6-bromo-2,3-dihydro-1H-inden-1-yl)oxy)-3-cyanophenyl)acetate (429b) (300 mg, 0.749 mmol) in dioxane/Me-THF (10 mL, ratio 9:1) using (1-aminoisoquinolin-7-yl)boronic acid (87a) (183 mg, 0.974 mmol). K₃PO₄ (4M aqueous solution, 749 μl, 3.00 mmol), PCy₃ (42.0 mg, 0.150 mmol), PdCl₂(dppf)-CH₂Cl₂ adduct (61.2 mg, 0.075 mmol) and Pd₂(dba)₃ (68.6 mg, 0.075 mmol) to afford after workup and purification using method-AE, (R)-ethyl 2-(2-((6-(1-aminoisoquinolin-7-yl)-2,3-dihydro-1H-inden-1-yl)oxy)-3-cyanophenyl)acetate (429c) (122 mg, 35% yield); MS (ES+): 464.2 (M+1); (ES−): 462.2 (M−1).

### Step-3: Preparation of (R)-2-(2-((6-(1-aminoisoquinolin-7-yl)-2,3-dihydro-1H-inden-1-yl)oxy)-3-cyanophenyl)acetic acid (429d)

Compound 429d was prepared according to the procedure reported in step-2 of scheme 1, from (R)-ethyl 2-(2-((6-(1-aminoisoquinolin-7-yl)-2,3-dihydro-1H-inden-1-yl)oxy)-3-cyanophenyl)acetate (429c) (122 mg, 0.263 mmol) in THF (5 mL) using lithium hydroxide hydrate (33.1 mg, 0.790 mmol) in water (1 mL) to afford after work up and purification using method-M, (R)-2-(2-((6-(1-aminoisoquinolin-7-yl)-2,3-dihydro-1H-inden-1-yl)oxy)-3-cyanophenyl)acetic acid (429d) (24.9 mg, 22% yield) HCl salt as a white solid; ¹H NMR (300 MHz, DMSO-d₆) δ 13.06 (s, 1H, D₂O Exchangeable), 12.44 (s, 1H, D₂O Exchangeable), 9.05 (s, 2H, D₂O Exchangeable), 8.84 (d, J=1.8 Hz, 1H), 8.10 (dd, J=8.5, 1.6 Hz, 1H), 8.01 (d, J=8.4 Hz, 1H), 7.89 (dd, J=7.9, 1.8 Hz, 1H), 7.78-7.65 (m, 2H), 7.65-7.53 (m, 3H), 7.32-7.20 (m, 2H), 5.88 (dd, J=5.3, 3.2 Hz, 1H), 3.60-3.40 (m, 2H), 3.29-3.22 (m, 1H), 3.05-2.89 (m, 1H), 2.44-2.28 (m, 2H); MS (ES+): 436.2 (M+1); Optical Rotation: −56.67 [MeOH, 0.12]; FT-1R: 2230.8 cm−1

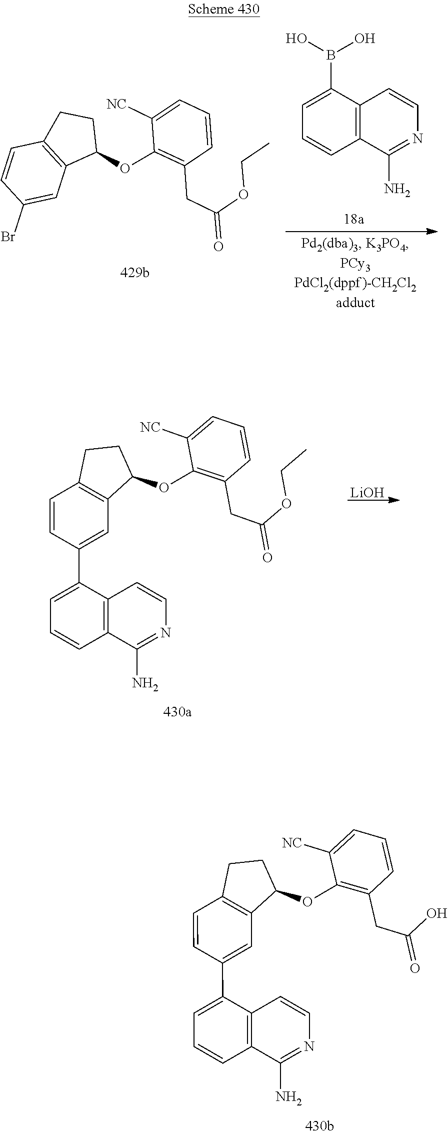

### Preparation of (R)-2-(2-((6-(1-aminoisoquinolin-5-yl)-2,3-dihydro-1H-inden-1-yl)oxy)-3-cyanophenyl)acetic acid (430b)

### Step-1: Preparation of (R)-ethyl 2-(2-((6-(1-aminoisoquinolin-5-yl)-2,3-dihydro-1H-inden-1-yl)oxy)-3-cyanophenyl)acetate (430a)

Compound 430a was prepared according to the procedure reported in step-5 of scheme 1, from (R)-ethyl 2-(2-((6-bromo-2,3-dihydro-1H-inden-1-yl)oxy)-3-cyanophenyl)acetate (429b) (300 mg, 0.749 mmol) in dioxane/Me-THF (10 mL, ratio 9:1) using (1-aminoisoquinolin-5-yl)boronic acid (18a) (183 mg, 0.974 mmol), K₃PO₄ (2 M aqueous solution, 636 mg, 3.00 mmol), PCy₃ (42.0 mg, 0.150 mmol), PdCl₂(dppf)-CH₂Cl₂ adduct (61.2 mg, 0.075 mmol) and Pd₂(dba)₃ (68.6 mg, 0.075 mmol) to afford after workup and purification using method-AE, (R)-ethyl 2-(2-((6-(1-aminoisoquinolin-5-yl)-2,3-dihydro-1H-inden-1-yl)oxy)-3-cyanophenyl)acetate (430a) (130 mg, 0.280 mmol, 37.4% yield); MS (ES+): 464.2 (M+1); (ES−): 462.0 (M−1).

### Step-2: Preparation of (R)-2-(2-((6-(1-aminoisoquinolin-5-yl)-2,3-dihydro-1H-inden-1-yl)oxy)-3-cyanophenyl)acetic acid (430b)

Compound 430b was prepared according to the procedure reported in step-2 of scheme 1, from (R)-ethyl 2-(2-((6-(1-aminoisoquinolin-5-yl)-2,3-dihydro-1H-inden-1-yl)oxy)-3-cyanophenyl)acetate (430a) (130 mg, 0.280 mmol) in THF (5 mL) using lithium hydroxide hydrate (35.3 mg, 0.841 mmol) in water (1 mL) to afford after work up and purification using method-M, (R)-2-(2-((6-(1-aminoisoquinolin-5-yl)-2,3-dihydro-1H-inden-1-yl)oxy)-3-cyanophenyl)acetic acid (430b) (32.1 mg, 26% yield) HCl salt as a white solid; ¹H NMR (300 MHz, DMSO-d₆) δ 13.19 (s, 1H, D₂O Exchangeable), 12.36 (s, 1H, D₂O Exchangeable), 9.09 (s, 2H, D₂O Exchangeable), 8.57 (d, J=8.2 Hz, 1H), 7.85-7.76 (m, 1H), 7.76-7.69 (m, 2H), 7.65-7.59 (m, 2H), 7.56 (d, J=7.8 Hz, 1H), 7.43-7.35 (m, 1H), 7.27 (t, J=7.7 Hz, 1H), 7.05 (d, J=1.7 Hz, 1H), 6.69 (d, J=7.2 Hz, 1H), 5.83 (t, J=3.9 Hz, 1H), 3.58-3.41 (m, 2H), 3.40-3.36 (m, 1H), 3.09-2.91 (m, 1H), 2.46-2.36 (m, 2H); MS (ES+): 436.2 (M+1); Optical Rotation: −48.755 [MeOH, 0.16]; FT-1R: 2230.8 cm·1.

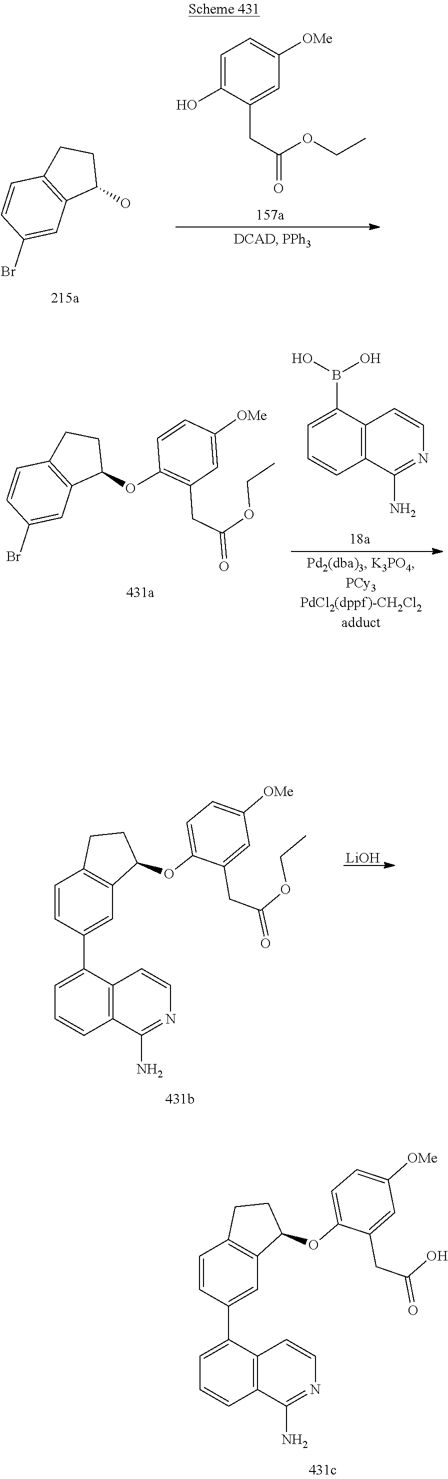

### Preparation of (R)-2-(2-((6-(1-aminoisoquinolin-5-yl)-2,3-dihydro-1H-inden-1-yl)oxy)-5-methoxyphenyl)acetic acid (431c)

### Step-1: Preparation of (R)-ethyl 2-(2-((6-bromo-2,3-dihydro-1H-inden-1-yl)oxy)-5-methoxyphenyl)acetate (431a)

Compound 431a was prepared according to the procedure reported in step-2 of scheme 2, from (S)-6-bromo-2,3-dihydro-1H-inden-1-ol (215a) (1.3 g, 6.10 mmol) in DCM (40 mL) using ethyl 2-(2-hydroxy-5-methoxyphenyl)acetate (157a) (1.283 g, 6.10 mmol). PPh₃ (1.760 g, 6.71 mmol) and a solution of DCAD (2.464 g, 6.71 mmol) in DCM (8 mL) to afford after workup and purification using method-BR. (R)-ethyl 2-(2-((6-bromo-2,3-dihydro-1H-inden-1-yl)oxy)-5-methoxyphenyl)acetate (431a) (1.345 g, 54% yield) as a white solid; MS (ES+): 405.0 (M+1); (ES−): 403.1 (M−1).

### Step-2: Preparation of (R)-ethyl 2-(2-((6-(1-aminoisoquinolin-5-yl)-2,3-dihydro-1H-inden-1-yl)oxy)-5-methoxyphenyl)acetate (431b)

Compound 431b was prepared according to the procedure reported in step-5 of scheme 1, from (R)-ethyl 2-(2-((6-bromo-2,3-dihydro-1H-inden-1-yl)oxy)-5-methoxyphenyl)acetate (431a) (300 mg, 0.740 mmol) in dioxane/Me-THF (10 mL, ratio 9:1) using (1-aminoisoquinolin-5-yl)boronic acid (18a) (181 mg, 0.962 mmol). K₃PO₄ (2M aqueous solution, 628 mg, 2.96 mmol), PCy₃ (41.5 mg, 0.148 mmol). PdCl₂(dppf)-CH₂Cl₂ adduct (60.4 mg, 0.074 mmol) and Pd₂(dba)₃ (67.8 mg, 0.074 mmol) to afford after workup and purification using method-AE. (R)-ethyl 2-(2-((6-(1-aminoisoquinolin-5-yl)-2,3-dihydro-1H-inden-1-yl)oxy)-5-methoxyphenyl)acetate (431b) (115 mg, 33% yield); MS (ES+): 469.2 (M+1).

### Step-3: Preparation of (R)-2-(2-((6-(1-aminoisoquinolin-5-yl)-2,3-dihydro-1H-inden-1-yloxy)-5-methoxyphenyl)acetic acid (431c)

Compound 431c was prepared according to the procedure reported in step-2 of scheme 1, from (R)-ethyl 2-(2-((6-(1-aminoisoquinolin-5-yl)-2,3-dihydro-1H-inden-1-yl)oxy)-5-methoxyphenyl)acetate (431b) (115 mg, 0.245 mmol) in THF (5 mL) using lithium hydroxide hydrate (30.9 mg, 0.736 mmol) in water (1 mL) to afford after work up and purification using method-M. (R)-2-(2-((6-(1-aminoisoquinolin-5-yl)-2,3-dihydro-1H-inden-1-yl)oxy)-5-methoxyphenyl)acetic acid (431c) (13.2 mg, 12% yield) HCl salt as a pale yellow solid; ¹H NMR (300 MHz, DMSO-d₆) δ 13.15 (s, 1H, D₂O Exchangeable), 12.01 (s, 1H, D₂O Exchangeable), 9.08 (s, 2H, D₂O Exchangeable), 8.58 (dd, J=7.8, 1.8 Hz, 1H), 7.91-7.78 (m, 2H), 7.58 (d, J=7.2 Hz, 1H), 7.50 (d, J=7.7 Hz, 1H), 7.39-7.30 (m, 2H), 7.17-7.09 (m, 1H), 6.90 (d, J=7.2 Hz, 1H), 6.85-6.75 (m, 2H), 5.81-5.71 (m, 1H), 3.69 (s, 3H), 3.39 (d, J=4.9 Hz, 2H), 3.22-3.08 (m, 1H), 3.04-2.88 (m, 1H), 2.72-2.56 (m, 1H), 2.16-2.04 (m, 1H); MS (ES+): 441.2 (M+1); (ES−): 439.2 (M−1); Optical Rotation: −172.0 [MeOH, 0.05].

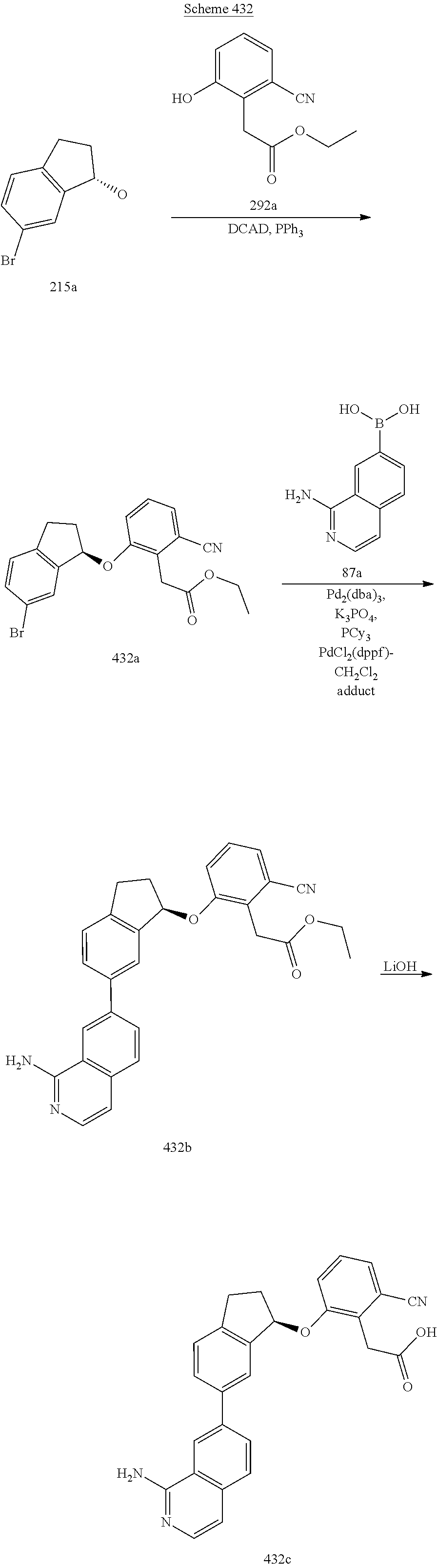

### Preparation of (R)-2-(2-((6-(1-aminoisoquinolin-7-yl)-2,3-dihydro-1H-inden-1-yl)oxy)-6-cyanophenyl)acetic acid (432c)

### Step-1: Preparation of (R)-ethyl 2-(2-((6-bromo-2,3-dihydro-1H-inden-1-yl)oxy)-6-cyanophenyl)acetate (432a)

Compound 432a was prepared according to the procedure reported in step-2 of scheme 2, from (S)-6-bromo-2,3-dihydro-1H-inden-1-ol (215a) (725 mg, 3.40 mmol) in DCM (40 mL) using ethyl 2-(2-cyano-6-hydroxyphenyl)acetate (292a) (698 mg, 3.40 mmol), PPh₃ (982 mg, 3.74 mmol) and a solution of DCAD (1374 mg, 3.74 mmol) in DCM (8 mL) to afford after workup and purification using method-BR, (R)-ethyl 2-(2-((6-bromo-2,3-dihydro-1H-inden-1-yl)oxy)-6-cyanophenyl)acetate (432a) (945 mg, 69% yield) as an off-white solid; MS (ES+): 422.1 (M+Na); (ES−): 398.1 (M−1).

### Step-2: Preparation of (R)-ethyl 2-(2-((6-(1-aminoisoquinolin-7-yl)-2,3-dihydro-1H-inden-1-yl)oxy)-6-cyanophenyl)acetate (432b)

Compound 432b was prepared according to the procedure reported in step-S of scheme 1, from (R)-ethyl 2-(2-((6-bromo-2,3-dihydro-1H-inden-1-yl)oxy)-6-cyanophenyl)acetate (432a) (300 mg, 0.749 mmol) in dioxane/Me-THF (10 mL, ratio 9:1) using (1-aminoisoquinolin-7-yl)boronic acid (87a) (183 mg, 0.974 mmol), K₃PO₄ (4M aqueous solution, 749 μl, 3.00 mmol), PCy₃ (42.0 mg, 0.15 mmol), PdCl₂(dppf)-CH₂Cl₂ adduct (61.2 mg, 0.075 mmol) and Pd₂(dba)₃ (68.6 mg, 0.075 mmol) to afford after workup and purification using method-AE, (R)-ethyl 2-(2-((6-(1-aminoisoquinolin-7-yl)-2,3-dihydro-1H-inden-1-yl)oxy)-6-cyanophenyl)acetate (432b) (129 mg, 37% yield); MS (ES+): 464.2 (M+1); (ES−): 462.1 (M−1).

### Step-3: Preparation of (R)-2-(2-((6-(1-aminoisoquinolin-7-yl)-2,3-dihydro-1H-inden-1-yl)oxy)-6-cyanophenyl)acetic acid (432c)

Compound 432c was prepared according to the procedure reported in step-2 of scheme 1, from (R)-ethyl 2-(2-((6-(1-aminoisoquinolin-7-yl)-2,3-dihydro-1H-inden-1-yl)oxy)-6-cyanophenyl)acetate (432b) (129 mg, 0.278 mmol) in THF (5 mL) using lithium hydroxide hydrate (35.0 mg, 0.835 mmol) in water (1 mL) to afford after work up and purification using method-M, (R)-2-(2-((6-(1-aminoisoquinolin-7-yl)-2,3-dihydro-1H-inden-1-yl)oxy)-6-cyanophenyl)acetic acid (432c) (30.3 mg, 25% yield) HCl salt as a white solid; ¹H NMR (300 MHz, DMSO-d₆) δ 12.85 (s, 1H, D₂O Exchangeable), 8.99 (s, 2H, D₂O Exchangeable), 8.88 (s, 1H), 8.29 (dd, J=8.5, 1.6 Hz, 1H), 8.03 (d, J=8.5 Hz, 1H), 7.92-7.86 (m, 2H), 7.73-7.65 (m, 2H), 7.56-7.48 (m, 2H), 7.44 (dd, J=7.7, 1.1 Hz, 1H), 7.26 (d, J=6.9 Hz, 1H), 6.08-5.96 (m, 1H), 3.68 (d, J=2.9 Hz, 2H), 3.19-3.04 (m, 1H), 3.05-2.88 (m, 1H), 2.79-2.63 (m, 1H), 2.11-1.96 (m, 1H); MS (ES+): 436.2 (M+1); (ES−): 434.2 (M−1); Optical Rotation: −160.0 [MeOH, 0.15]; FT-1R: 2227.1 cm⁻¹.

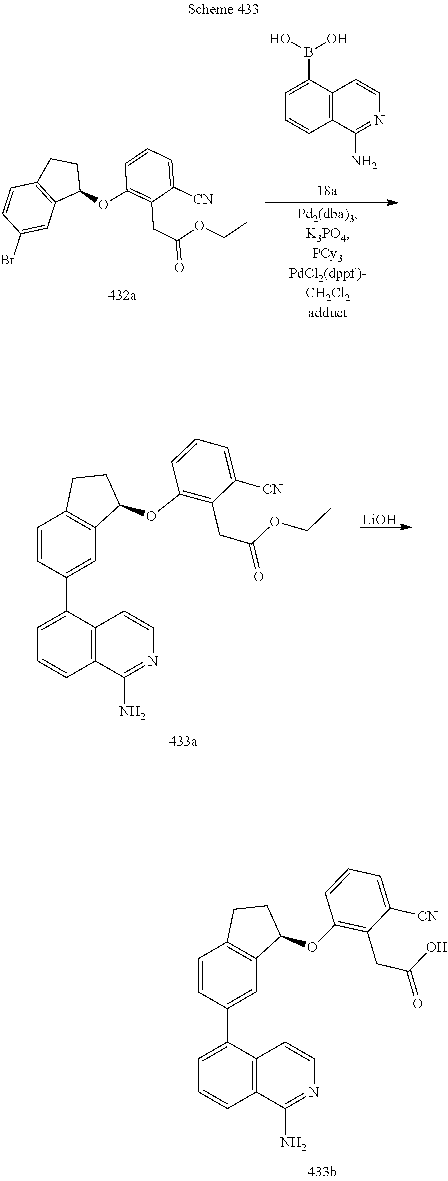

### Preparation of (R)-2-(2-((6-(1-aminoisoquinolin-5-yl)-2,3-dihydro-1H-inden-1-yl)oxy)-6-cyanophenyl)acetic acid (433b)

### Step-1: Preparation of (R)-ethyl 2-(2-((6-(1-aminoisoquinolin-5-yl)-2,3-dihydro-1H-inden-1-yl)oxy)-6-cyanophenyl)acetate (433a)

Compound 433a was prepared according to the procedure reported in step-5 of scheme 1, from (R)-ethyl 2-(2-((6-bromo-2,3-dihydro-1H-inden-1-yl)oxy)-6-cyanophenyl)acetate (432a) (300 mg, 0.749 mmol) in dioxane/Me-THF (10 mL, ratio 9:1) using (1-aminoisoquinolin-S-yl)boronic acid (18a) (183 mg, 0.974 mmol), K₃PO₄ (2 M aqueous solution, 636 mg, 3.00 mmol), PCy₃ (42.0 mg, 0.150 mmol), PdCl₂(dppf)-CH₂Cl₂ adduct (61.2 mg, 0.075 mmol) and Pd₂(dba)₃ (68.6 mg, 0.075 mmol) to afford after workup and purification using method-AE, (R)-ethyl 2-(2-((6-(1-aminoisoquinolin-5-yl)-2,3-dihydro-1H-inden-1-yl)oxy)-6-cyanophenyl)acetate (433a) (114 mg, 33% yield); MS (ES+): 464.2 (M+1).

### Step-2: Preparation of (R)-2-(2-((6-(1-aminoisoquinolin-5-yl)-2,3-dihydro-1H-inden-1-yl)oxy)-6-cyanophenyl)acetic acid (433b)

Compound 433b was prepared according to the procedure reported in step-2 of scheme 1, from (R)-ethyl 2-(2-((6-(1-aminoisoquinolin-5-yl)-2,3-dihydro-1H-inden-1-yl)oxy)-6-cyanophenyl)acetate (433a) (114 mg, 0.246 mmol) in THF (5 mL) using lithium hydroxide hydrate (31.0 mg, 0.738 mmol) in water (1 mL) to afford after work up and purification using method-M, (R)-2-(2-((6-(1-aminoisoquinolin-5-yl)-2,3-dihydro-1H-inden-1-yl)oxy)-6-cyanophenyl)acetic acid (433b) (30.1 mg, 28% yield) HCl salt as a pale yellow solid; 1H NMR (300 MHz, DMSO-d₆) δ 13.21 (s, 1H, D₂O Exchangeable), 12.45 (s, 1H, D₂O Exchangeable), 9.10 (s, 2H, D₂O Exchangeable), 8.65-8.54 (m, 1H), 7.91-7.85 (m, 1H), 7.85-7.80 (m, 1H), 7.68-7.59 (m, 2H), 7.55-7.45 (m, 2H), 7.45-7.37 (m, 3H), 6.93 (d, J=7.2 Hz, 1H), 6.01 (t, J=5.7 Hz, 1H), 3.66 (d, J=2.6 Hz, 2H), 3.22-3.09 (m, 1H), 3.08-2.91 (m, 1H), 2.82-2.68 (m, 1H), 2.14-2.00 (m, 1H); MS (ES+): 436.2 (M+1); Optical Rotation: −137.14 [MeOH, 0.105]; FT-1R: 2227.1 cm⁻¹.

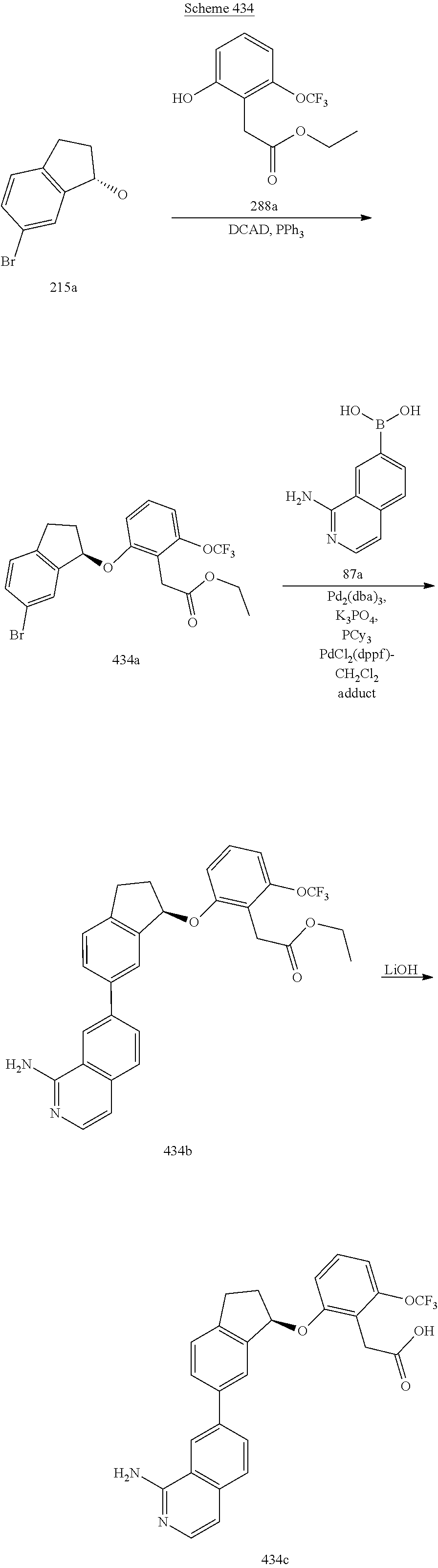

### Preparation of (R)-2-(2-((6-(1-aminoisoquinolin-7-yl)-2,3-dihydro-1H-inden-1-yl)oxy)-6-(trifluoromethoxy)phenyl)acetic acid (434c)

### Step-1: Preparation of (R)-ethyl 2-(2-((6-bromo-2,3-dihydro-1H-inden-1-yl)oxy)-6-(trifluoromethoxy)phenyl)acetate (434a)

Compound 434a was prepared according to the procedure reported in step-2 of scheme 2, from (S)-6-bromo-2,3-dihydro-1H-inden-1-ol (215a) (1.5 g, 7.04 mmol) in DCM (40 mL) using ethyl 2-(2-hydroxy-6-(trifluoromethoxy)phenyl)acetate (288a) (1.860 g, 7.04 mmol), PPh₃ (2.031 g, 7.74 mmol) and a solution of DCAD (2.84 g, 7.74 mmol) in DCM (8 mL) to afford after workup and purification using method-BR. (R)-ethyl 2-(2-((6-bromo-2,3-dihydro-1H-inden-1-yl)oxy)-6-(trifluoromethoxy)phenyl)acetate (434a) (1.213 g, 2.64 mmol, 37.5% yield) as a white solid; MS (ES+): 481.00 (M+Na).

### Step-2: Preparation of (R)-ethyl 2-(2-((6-(1-aminoisoquinolin-7-yl)-2,3-dihydro-1H-inden-1-yl)oxy)-6-(trifluoromethoxy)phenyl)acetate (434b)

Compound 434b was prepared according to the procedure reported in step-5 of scheme 1, from (R)-ethyl 2-(2-((6-bromo-2,3-dihydro-1H-inden-1-yl)oxy)-6-(trifluoromethoxy)phenyl)acetate (434a) (300 mg, 0.653 mmol) in dioxane/Me-THF (10 mL, ratio 9:1) using (1-aminoisoquinolin-7-yl)boronic acid (87a) (160 mg, 0.849 mmol), K₃PO₄ (4M aqueous solution, 653 μl, 2.61 mmol), PCy₃ (36.6 mg, 0.131 mmol), PdCl₂(dppf)-CH₂Cl₂ adduct (53.3 mg, 0.065 mmol) and Pd₂(dba)₃ (59.8 mg, 0.065 mmol) to afford after workup and purification using method-AE, (R)-ethyl 2-(2-((6-(1-aminoisoquinolin-7-yl)-2,3-dihydro-1H-inden-1-yl)oxy)-6-(trifluoromethoxy)phenyl)acetate (434b) (127 mg, 37% yield); MS (ES+): 523.2 (M+1); (ES−): 521.1 (M−1).

### Step-3: Preparation of (R)-2-(2-((6-(1-aminoisoquinolin-7-yl)-2,3-dihydro-1H-inden-1-yl)oxy)-6-(trifluoromethoxy)phenyl)acetic acid (434c)

Compound 434c was prepared according to the procedure reported in step-2 of scheme 1, from (R)-ethyl 2-(2-((6-(1-aminoisoquinolin-7-yl)-2,3-dihydro-1H-inden-1-yl)oxy)-6-(trifluoromethoxy)phenyl)acetate (434b) (127 mg, 0.243 mmol) in THF (5 mL) using lithium hydroxide hydrate (30.6 mg, 0.729 mmol) in water (1 mL) to afford after work up and purification using method-M. (R)-2-(2-((6-(1-aminoisoquinolin-7-yl)-2,3-dihydro-1H-inden-1-yl)oxy)-6-(trifluoromethoxy)phenyl)acetic acid (434c) (19.1 mg, 0.039 mmol, 15.89% yield) HCl salt as a white solid; ¹H NMR (300 MHz, DMSO-d₆) δ 12.94 (s, 1H, D₂O Exchangeable), 12.45 (s, 1H, D₂O Exchangeable), 8.98 (s, 2H, D₂O Exchangeable), 8.87 (s, 1H), 8.30 (dd. J=8.5, 1.7 Hz, 1H), 8.03 (d, J=8.5 Hz, 1H), 7.94-7.83 (m, 2H), 7.69 (d, J=6.9 Hz, 1H), 7.53 (d, J=8.3 Hz, 1H), 7.45 (t, J=8.3 Hz, 1H), 7.40-7.33 (m, 1H), 7.26 (d, J=6.9 Hz, 1H), 7.00 (dt, J=8.3, 1.5 Hz, 1H), 6.00 (t, 1H), 3.52 (d, J=2.2 Hz, 2H), 3.20-3.04 (m, 1H), 3.05-2.87 (m, 1H), 2.80-2.61 (m, 1H), 2.15-1.96 (m, 1H); ¹⁹F NMR (282 MHz, DMSO-d₆) δ −56.08 (d, J=1.8 Hz); MS (ES+): 495.2 (M+1); (ES−): 493.2 (M−1); Optical Rotation: −131.43 [MeOH, 0.105].

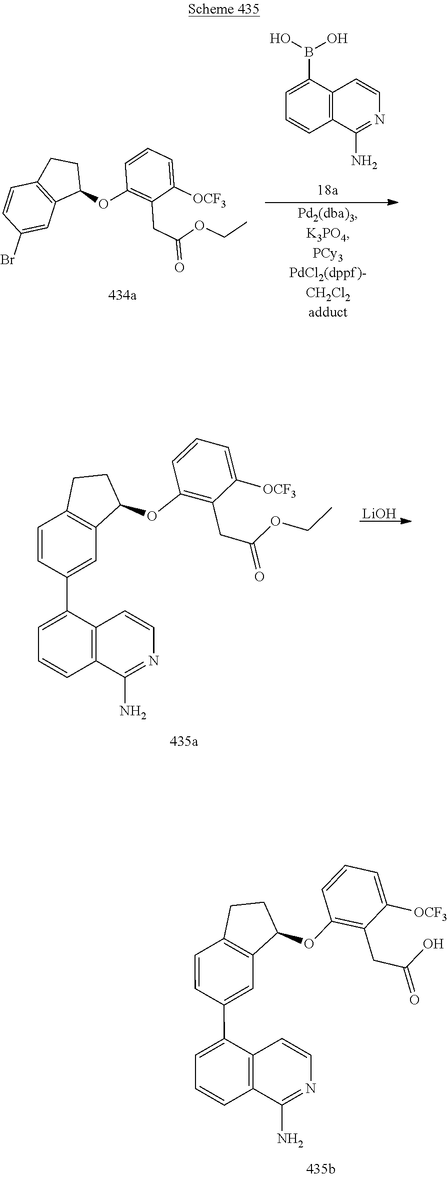

### Preparation of (R)-2-(2-((6-(1-aminoisoquinolin-5-yl)-2,3-dihydro-1H-inden-1-yl)oxy)-6-(trifluoromethoxy)phenyl)acetic acid (435b)

### Step-1: Preparation of (R)-ethyl 2-(2-((6-(1-aminoisoquinolin-5-yl)-2,3-dihydro-1H-inden-1-yl)oxy)-6-(trifluoromethoxy)phenyl)acetate (435a)

Compound 435a was prepared according to the procedure reported in step-5 of scheme 1, from (R)-ethyl 2-(2-((6-bromo-2,3-dihydro-1H-inden-1-yl)oxy)-6-(trifluoromethoxy)phenyl)acetate (434a) (300 mg, 0.653 mmol) in dioxane/Me-THF (10 mL, ratio 9:1) using (1-aminoisoquinolin-5-yl)boronic acid (18a) (160 mg, 0.849 mmol). K₃PO₄ (2 M aqueous solution, 555 mg, 2.61 mmol), PCy₃ (36.6 mg, 0.131 mmol). PdCl₂(dppf)-CH₂Cl₂ adduct (53.3 mg, 0.065 mmol) and Pd₂(dba)₃ (59.8 mg, 0.065 mmol) to afford after workup and purification using method-AE, (R)-ethyl 2-(2-((6-(1-aminoisoquinolin-5-yl)-2,3-dihydro-1H-inden-1-yl)oxy)-6-(trifluoromethoxy)phenyl)acetate (435a) (136 mg, 40% yield); MS (ES+): 523.2 (M+1); (ES−): 521.0 (M−1).

### Step-2: Preparation of (R)-2-(2-((6-(1-aminoisoquinolin-5-yl)-2,3-dihydro-1H-inden-1-yl)oxy)-6-(trifluoromethoxy)phenyl)acetic acid (435b)

Compound 435b was prepared according to the procedure reported in step-2 of scheme 1, from (R)-ethyl 2-(2-((6-(1-aminoisoquinolin-5-yl)-2,3-dihydro-1H-inden-1-yl)oxy)-6-(trifluoromethoxy)phenyl)acetate (435a) (136 mg, 0.260 mmol) in THF (5 mL) using lithium hydroxide hydrate (32.8 mg, 0.781 mmol) in water (1 mL) to afford after work up and purification using method-M, (R)-2-(2-((6-(1-aminoisoquinolin-5-yl)-2,3-dihydro-1H-inden-1-yl)oxy)-6-(trifluoromethoxy)phenyl)acetic acid (435b) (40.0 mg, 0.081 mmol, 31.1% yield) HCl salt as an off-white solid; ¹H NMR (300 MHz, DMSO-d₆) δ 13.12 (s, 1H, D₂O Exchangeable), 12.21 (s, 1H, D₂O Exchangeable), 9.04 (s, 2H, D₂O Exchangeable), 8.58 (dd, J=7.9, 1.4 Hz, 1H), 7.91-7.80 (m, 2H), 7.60 (d, J=7.2 Hz, 1H), 7.52 (d, J=7.7 Hz, 1H), 7.44-7.36 (m, 3H), 7.36-7.30 (m, 1H), 7.01-6.92 (m, 2H), 5.98 (t, J=5.8 Hz, 1H), 3.50 (s, 2H), 3.22-3.09 (m, 1H), 3.07-2.91 (m, 1H), 2.83-2.68 (m, 1H), 2.15-2.01 (m, 1H); ¹⁹F NMR (282 MHz, DMSO-d₆) δ −56.10 (d, J=1.9 Hz); MS (ES+): 495.2 (M+1); (ES−): 493.1 (M−1); Optical Rotation: −66.67 [MeOH, 0.06].

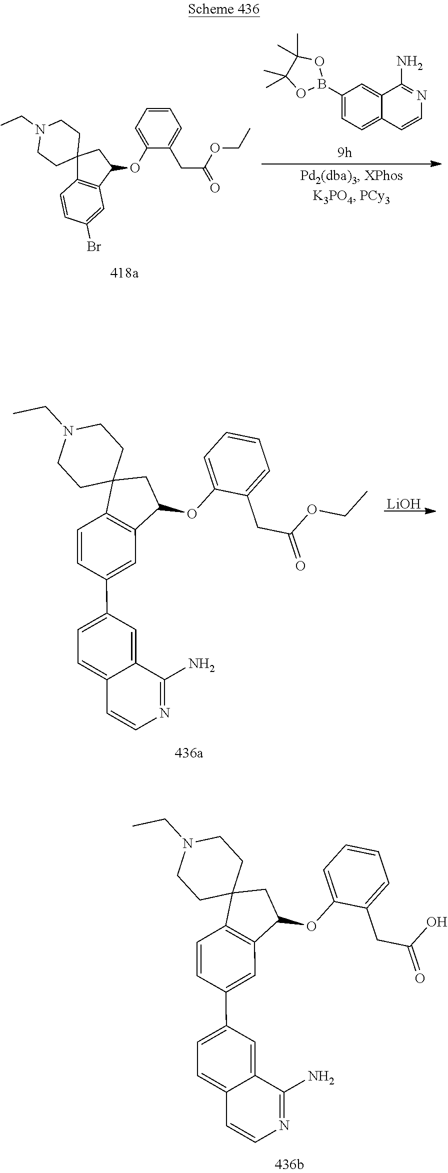

### Preparation of (R)-2-(2-((5-(1-aminoisoquinolin-7-yl)-1′-ethyl-2,3-dihydrospiro[indene-1,4′-piperidin]-3-yl)oxy)phenyl)acetic acid (436b)

### Step-1: Preparation of (R)-ethyl 2-(2-((5-(1-aminoisoquinolin-7-yl)-1′-ethyl-2,3-dihydrospiro[indene-1,4′-piperidin]-3-yl)oxy)phenyl)acetate (436a)

Compound 436a was prepared according to the procedure reported in step-3 of scheme 112, from (R)-ethyl 2-(2-((5-bromo-1′-ethyl-2,3-dihydrospiro[indene-1,4′-piperidin]-3-yl)oxy)phenyl)acetate (418a) (85 mg, 0.180 mmol) in dioxane/2-MeTHF (2 mL) using 7-(4,4,5,5-tetramethyl-1,3,2-dioxaborolan-2-yl)isoquinolin-1-amine (9h) (97 mg, 0.36 mmol), K₃PO₄ (3M aqueous solution, 120 μl, 0.36 mmol). PCy₃ (20.18 mg, 0.072 mmol). XPhos (17.15 mg, 0.036 mmol), Pd₂(dba)₃ (33.0 mg, 0.036 mmol) and heating at 100° C. for 10 h to afford after workup and purification using method-U, (R)-ethyl 2-(2-((5-(1-aminoisoquinolin-7-yl)-1′-ethyl-2,3-dihydrospiro[indene-1,4′-piperidin]-3-yl)oxy)phenyl)acetate (436a) (56 mg, 58% yield) as a clear oil; MS (ES+): 536.4 (M+1).

### Step-2: Preparation of (R)-2-(2-((5-(1-aminoisoquinolin-7-yl)-1′-ethyl-2,3-dihydrospiro[indene-1,4′-piperidin]-3-yl)oxy)phenyl)acetic acid (436b)

Compound 436b was prepared according to the procedure reported in step-2 of scheme 1, from (R)-ethyl 2-(2-((5-(1-aminoisoquinolin-7-yl)-1′-ethyl-2,3-dihydrospiro[indene-1,4′-piperidin]-3-yl)oxy)phenyl)acetate (436a) (56 mg, 0.105 mmol) in THF (3 mL) using lithium hydroxide (12.93 mg, 0.540 mmol) in water (1 mL) to afford after work up and purification using method-M, (R)-2-(2-((5-(1-aminoisoquinolin-7-yl)-1′-ethyl-2,3-dihydrospiro[indene-1,4′-piperidin]-3-yl)oxy)phenyl)acetic acid (436b) (10 mg, 11% yield) HCl salt as a white solid; ¹H NMR (300 MHz, DMSO-d₆) δ 11.98 (s, 1H, D₂O exchangeable), 10.21 (s, 1H, D₂O exchangeable), 8.82 (s, 1H), 8.61 (s, 2H, D₂O exchangeable), 8.24 (d, J=8.6 Hz, 1H), 8.03-7.93 (m, 2H), 7.92 (d, J=1.7 Hz, 1H), 7.72 (d, J=6.7 Hz, 1H), 7.41 (d, J=8.1 Hz, 1H), 7.36-7.27 (m, 2H), 7.26-7.15 (m, 2H), 7.00-6.90 (m, 1H), 6.03-5.92 (m, 1H), 3.56-3.40 (m, 4H), 3.21-3.08 (m, 3H), 3.05-2.93 (m, 1H), 2.81-2.67 (m, 1H), 2.42-2.24 (m, 2H), 2.22-2.08 (m, 1H), 1.90-1.72 (m, 2H), 1.29 (t, J=7.2 Hz, 3H); MS (ES+): 508.2 (M+1); (ES−): 506.3 (M−1).

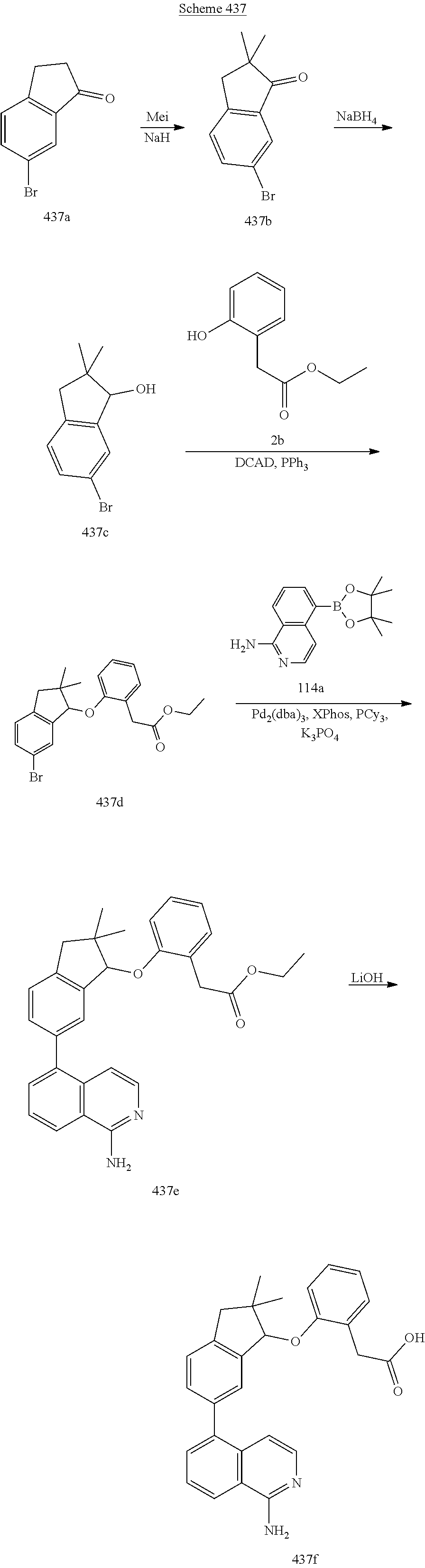

### Preparation of 2-(2-((6-(1-aminoisoquinolin-5-yl)-2,2-dimethyl-2,3-dihydro-1H-inden-1-yl)oxy)phenyl)acetic acid (437f)

### Step-1: Preparation of 6-bromo-2,2-dimethyl-2,3-dihydro-1H-inden-1-one (437b)

To a solution of 6-bromo-2,3-dihydro-1H-inden-1-one (437a) (15 g, 71.1 mmol) in THF (300 mL) was added methyl iodide (11.11 mL, 178 mmol) stirred for 20 min followed by the addition of sodium hydride (60% dispersion in mineral oil) (7.11 g, 178 mmol) in portions and stirred for 12 h at RT. The reaction mixture was cooled to 0° C. quenched with water and extracted with EtOAc (3×300 mL). Combined organics were washed with brine, dried, filtered and concentrated. The residue obtained was purified using method-P, to afford 6-bromo-2,2-dimethyl-2,3-dihydro-1H-inden-1-one (437b) (16.03 g, 94% yield) as a yellow oil; MS (ES+): 239.0 and 241.0 (M+1).

### Step-2: Preparation of 6-bromo-2,2-dimethyl-2,3-dihydro-1H-inden-1-ol (437c)

Compound 437c was prepared according to the procedure reported in step-1 of scheme 205, from 6-bromo-2,2-dimethyl-2,3-dihydro-1H-inden-1-one (437b) (12 g, 50.2 mmol) in anhydrous MeOH (20 mL) using sodium borohydride (2.85 g, 75 mmol) to afford after work up and purification method-J, 6-bromo-2,2-dimethyl-2,3-dihydro-1H-inden-1-ol (437c) (8.4 g, 69% yield) as a white solid; ¹H NMR (300 MHz, DMSO-d₆) δ 7.38 (d, J=1.9 Hz, 1H), 7.32 (dd. J=7.9, 2.0 Hz, 1H), 7.12 (d, J=7.9 Hz, 1H), 5.34 (d, J=6.0 Hz, 1H), 4.55 (d, J=6.0 Hz, 1H), 2.56 (d, J=6.6 Hz, 2H), 1.12 (s, 3H), 0.84 (s, 3H).

### Step-3: Preparation of ethyl 2-(2-((6-bromo-2,2-dimethyl-2,3-dihydro-1H-inden-1-yl)oxy)phenyl)acetate (437d)

Compound 437d was prepared according to the procedure reported in step-2 of scheme 2, from 6-bromo-2,2-dimethyl-2,3-dihydro-1H-inden-1-ol (437c) (1 g, 4.15 mmol) in DCM (30 mL) using ethyl 2-(2-hydroxyphenyl)acetate (2b) (0.747 g, 4.15 mmol), PPh₃ (1.088 g, 4.15 mmol) and a solution of DCAD (1.523 g, 4.15 mmol) in DCM (60 mL) to afford after workup and purification using method-V, ethyl 2-(2-((6-bromo-2,2-dimethyl-2,3-dihydro-1H-inden-1-yl)oxy)phenyl)acetate (437d) (480 mg, 29% yield) as clear oil; MS (ES+): 425.1 and 427.0 (M+Na).

### Step-4: Preparation of ethyl 2-(2-((6-(1-aminoisoquinolin-5-yl)-2,2-dimethyl-2,3-dihydro-1H-inden-1-yl)oxy)phenyl)acetate (437e)

Compound 437e was prepared according to the procedure reported in step-5 of scheme 1, from ethyl 2-(2-((6-bromo-2,2-dimethyl-2,3-dihydro-1H-inden-1-yl)oxy)phenyl)acetate (437d) (160 mg, 0.397 mmol) in dioxane/2Me-THF (2 mL) using 5-(4,4,5,5-tetramethyl-1,3,2-dioxaborolan-2-yl)isoquinolin-1-amine (114a) (214 mg, 0.793 mmol), K₃PO₄ (3M aqueous solution, 264 μl, 0.793 mmol), PCy₃ (44.5 mg, 0.159 mmol). Pd₂(dba)₃ (72.7 mg, 0.079 mmol) and XPhos (37.8 mg, 0.079 mmol) to afford after workup and purification using method-U, ethyl 2-(2-((6-(1-aminoisoquinolin-5-yl)-2,2-dimethyl-2,3-dihydro-1H-inden-1-yl)oxy)phenyl)acetate (437e) (96 mg, 52% yield) as a clear oil; MS (ES+): 467.3 (M+1); (ES−): 465.3 (M−1).

### Step-5: Preparation of 2-(2-((6-(1-aminoisoquinolin-5-yl)-2,2-dimethyl-2,3-dihydro-1H-inden-1-yl)oxy)phenyl)acetic acid (437f)

Compound 437f was prepared according to the procedure reported in step-2 of scheme 1, from ethyl 2-(2-((6-(1-aminoisoquinolin-5-yl)-2,2-dimethyl-2,3-dihydro-1H-inden-1-yl)oxy)phenyl)acetate (437e) (96 mg, 0.206 mmol) in THF (3 mL) using lithium hydroxide (28.5 mg, 1.190 mmol) in water (1 mL) to afford after workup and purification method-M, 2-(2-((6-(1-aminoisoquinolin-5-yl)-2,2-dimethyl-2,3-dihydro-1H-inden-1-yl)oxy)phenyl)acetic acid (4371) (66 mg, 38% yield) HCl salt as a white solid; ¹H NMR (300 MHz, DMSO-d₆) δ 13.38 (s, 1H, D₂O exchangeable), 12.05 (s, 1H, D₂O exchangeable), 9.18 (s, 2H, D₂O exchangeable), 8.57 (dd, J=8.0, 1.7 Hz, 1H), 7.87-7.72 (m, 2H), 7.58 (d, J=7.3 Hz, 1H), 7.45 (d, J=7.7 Hz, 1H), 7.35-7.15 (m, 4H), 7.14-7.09 (m, 1H), 6.91-6.86 (m, 1H), 6.86-6.78 (m, 1H), 5.53 (s, 1H), 3.43 (d, J=2.6 Hz, 2H), 2.88 (q, J==15.6 Hz, 2H), 1.24 (s, 3H), 1.13 (s, 3H); MS (ES+): 439.2 (M+1); (ES−): 437.3 (M−1).

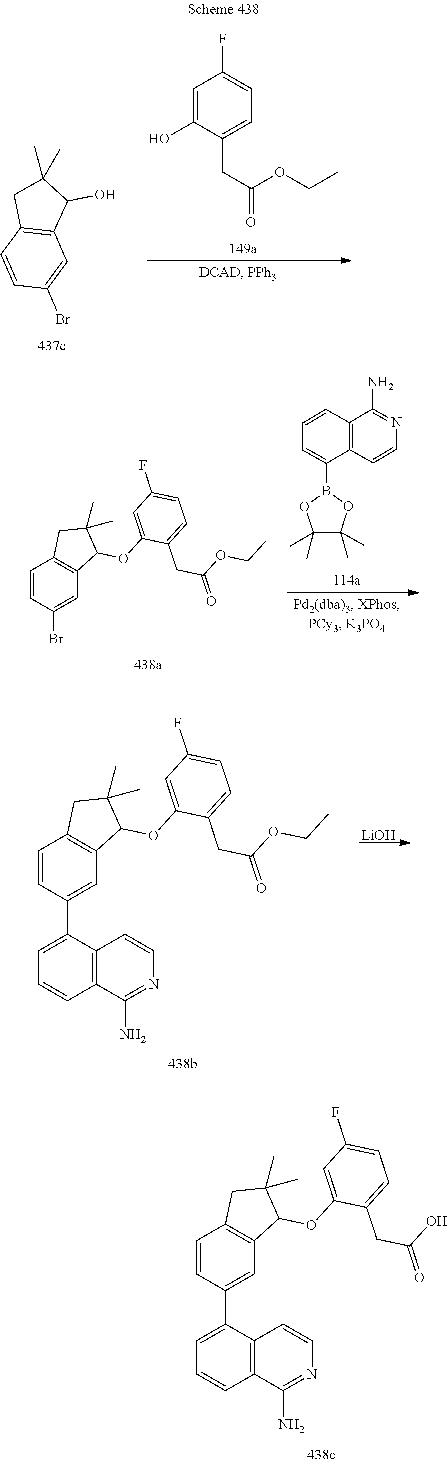

### Preparation of 2-(2-((6-(1-aminoisoquinolin-5-yl)-2,2-dimethyl-2,3-dihydro-1H-inden-1-yl)oxy)-4-fluorophenyl)acetic acid (438c)

### Step-1: Preparation of ethyl 2-(2-((6-bromo-2,2-dimethyl-2,3-dihydro-1H-inden-1-yl)oxy)-4-fluorophenyl)acetate (438a)

Compound 438a was prepared according to the procedure reported in step-2 of scheme 2, from 6-bromo-2,2-dimethyl-2,3-dihydro-1H-inden-1-ol (437c) (1 g, 4.15 mmol) in DCM (30 mL) using ethyl 2-(4-fluoro-2-hydroxyphenyl)acetate (149a) (0.822 g, 4.15 mmol), PPh₃ (1.088 g, 4.15 mmol) and a solution of DCAD (1.523 g, 4.15 mmol) in DCM (60 mL) to afford after workup and purification using method-V, ethyl 2-(2-((6-bromo-2,2-dimethyl-2,3-dihydro-1H-inden-1-yl)oxy)-4-fluorophenyl)acetate (438a) (600 mg, 34% yield) as a clear oil; MS (ES+): 421.1 and 423.1 (M+1); (ES−): 419.0 and 421.0 (M−1).

### Step-2: Preparation of ethyl 2-(2-((6-(1-aminoisoquinolin-5-yl)-2,2-dimethyl-2,3-dihydro-1H-inden-1-yl)oxy)-4-fluorophenyl)acetate (438b)

Compound 438b was prepared according to the procedure reported in step-5 of scheme 1, from ethyl 2-(2-((6-bromo-2,2-dimethyl-2,3-dihydro-1H-inden-1-yl)oxy)-4-fluorophenyl)acetate (438a) 200 mg, 0.475 mmol) in dioxane/2Me-THF (2 mL) using 5-(4,4,5,5-tetramethyl-1,3,2-dioxaborolan-2-yl)isoquinolin-1-amine (114a) (256 mg, 0.949 mmol), K₃PO₄ (3M aqueous solution, 316 μl, 0.949 mmol), PCy₃ (53.3 mg, 0.190 mmol), Pd₂(dba)₃ (87 mg, 0.095 mmol) and XPhos (45.3 mg, 0.095 mmol) to afford after workup and purification using method-U, ethyl 2-(2-((6-(1-aminoisoquinolin-5-yl)-2,2-dimethyl-2,3-dihydro-1H-inden-1-yl)oxy)-4-fluorophenyl)acetate (438b) (121 mg, 53% yield) as a clear oil; MS (ES+): 485.3 (M+1); (ES−): 483.3 (M−1).

### Step-3: Preparation of 2-(2-((6-(1-aminoisoquinolin-5-yl)-2,2-dimethyl-2,3-dihydro-1H-inden-1-yl)oxy)-4-fluorophenyl)acetic acid (438c)

Compound 438c was prepared according to the procedure reported in step-2 of scheme 1, from ethyl 2-(2-((6-(1-aminoisoquinolin-5-yl)-2,2-dimethyl-2,3-dihydro-1H-inden-1-yl)oxy)-4-fluorophenyl)acetate (438b) (121 mg, 0.250 mmol) in THF (3 mL) using lithium hydroxide (34.1 mg, 1.424 mmol) in water (1 mL) to afford after workup and purification method-M, 2-(2-((6-(1-aminoisoquinolin-S-yl)-2,2-dimethyl-2,3-dihydro-1H-inden-1-yl)oxy)-4-fluorophenyl)acetic acid (438c) (86 mg, 40% yield) HCl salt as a white solid; ¹H NMR (300 MHz, DMSO-d₆) δ 13.46 (s, 1H, D₂O exchangeable), 12.09 (s, 1H, D₂O exchangeable), 9.22 (s, 2H, D₂O exchangeable), 8.59 (dd, J=7.9, 1.7 Hz, 1H), 7.90-7.68 (m, 2H), 7.56 (d, J=7.3 Hz, 1H), 7.45 (d, J=7.7 Hz, 1H), 7.32 (dd, J=7.7, 1.7 Hz, 1H), 7.27-7.17 (m, 2H), 7.17-7.10 (m, 1H), 6.83 (d, J=7.2 Hz, 1H), 6.71 (td, J=8.4, 2.4 Hz, 1H), 5.61 (s, 1H), 3.49-3.37 (m, 2H), 3.01-2.74 (m, 2H), 1.23 (s, 3H), 1.12 (s, 3H); ¹⁹F NMR (282 MHz, DMSO-de) 8-112.79; MS (ES+): 457.3 (M+1); (ES−) 455.2 (M−1).

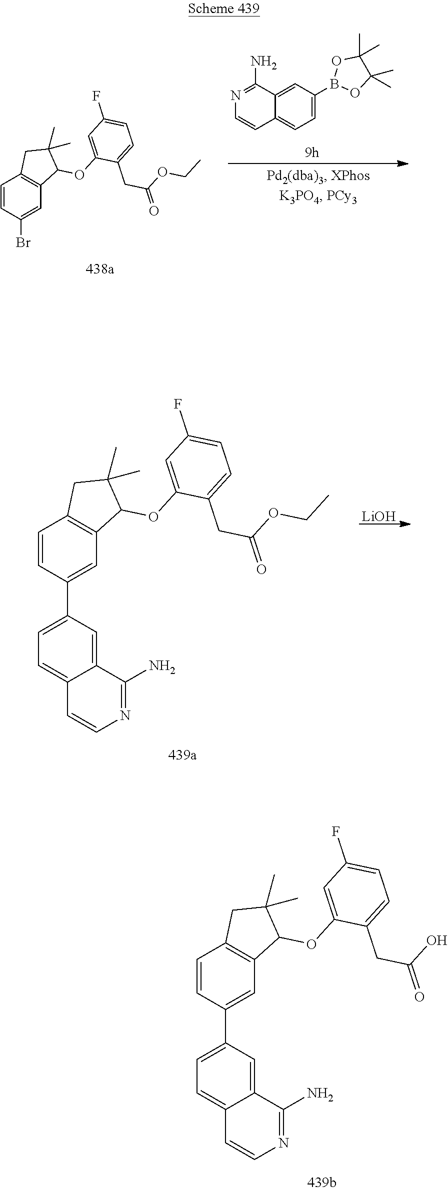

### Preparation of 2-(2-((6-(1-aminoisoquinolin-7-yl)-2,2-dimethyl-2,3-dihydro-1H-inden-1-yl)oxy)-4-fluorophenyl)acetic acid (439b)

### Step-1: Preparation of ethyl 2-(2-((6-(1-aminoisoquinolin-7-yl)-2,2-dimethyl-2,3-dihydro-1H-inden-1-yl)oxy)-4-fluorophenyl)acetate (439a)

Compound 439a was prepared according to the procedure reported in step-5 of scheme 1, from ethyl 2-(2-((6-bromo-2,2-dimethyl-2,3-dihydro-1H-inden-1-yl)oxy)-4-fluorophenyl)acetate (438a) (200 mg, 0.475 mmol) in dioxane/2Me-THF (2 mL) using 7-(4,4,5,5-tetramethyl-1,3,2-dioxaborolan-2-yl)isoquinolin-1-amine (9h) (256 mg, 0.949 mmol). K₃PO₄ (3M aqueous solution, 316 μl, 0.949 mmol), PCy₃ (53.3 mg, 0.190 mmol), Pd₂(dba)₃ (87 mg, 0.095 mmol) and XPhos (45.3 mg, 0.095 mmol) to afford after workup and purification using method-U, ethyl 2-(2-((6-(1-aminoisoquinolin-7-yl)-2,2-dimethyl-2,3-dihydro-1H-inden-1-yl)oxy)-4-fluorophenyl)acetate (439a) (55 mg, 0.114 mmol, 23.91% yield) as a clear oil; MS (ES+): 485.3 (M+1); (ES−): 483.2 (M−1).

### Step-2: Preparation of 2-(2-((6-(1-aminoisoquinolin-7-yl)-2,2-dimethyl-2,3-dihydro-1H-inden-1-yl)oxy)-4-fluorophenyl)acetic acid (439b)

Compound 439b was prepared according to the procedure reported in step-2 of scheme 1, from ethyl 2-(2-((6-(1-aminoisoquinolin-7-yl)-2,2-dimethyl-2,3-dihydro-1H-inden-1-yl)oxy)-4-fluorophenyl)acetate (439a) (55 mg, 0.114 mmol) in THF (3 mL) using lithium hydroxide (34.1 mg, 1.424 mmol) in water (1 mL) to afford after workup and purification method-M, 2-(2-((6-(1-aminoisoquinolin-7-yl)-2,2-dimethyl-2,3-dihydro-1H-inden-1-yl)oxy)-4-fluorophenyl)acetic acid (439b) (20 mg, 9% yield) HCl salt as an off-white solid; ¹H NMR (300 MHz, DMSO-d₆) δ 13.15 (s, 1H, D₂O exchangeable), 12.12 (s, 1H, DO exchangeable), 9.06 (s, 2H, D₂O exchangeable), 8.81 (d, J=1.7 Hz, 1H), 8.17 (dd, J=8.5, 1.7 Hz, 1H), 8.00 (d, J=8.5 Hz, 1H), 7.78 (dd, J=7.8, 1.8 Hz, 1H), 7.67 (d, J=6.9 Hz, 1H), 7.59 (d, J=1.7 Hz, 1H), 7.46 (d, J=7.9 Hz, 1H), 7.28-7.17 (m, 3H), 6.78-6.66 (m, 1H), 5.65 (s, 1H), 3.46 (s, 2H), 2.85 (q, J=15.6 Hz, 2H), 1.23 (s, 3H), 1.08 (s, 3H); ¹⁹F NMR (282 MHz, DMSO-d₆) δ −112.69; MS (ES+): 457.2 (M+1); (ES−): 455.2 (M−1).

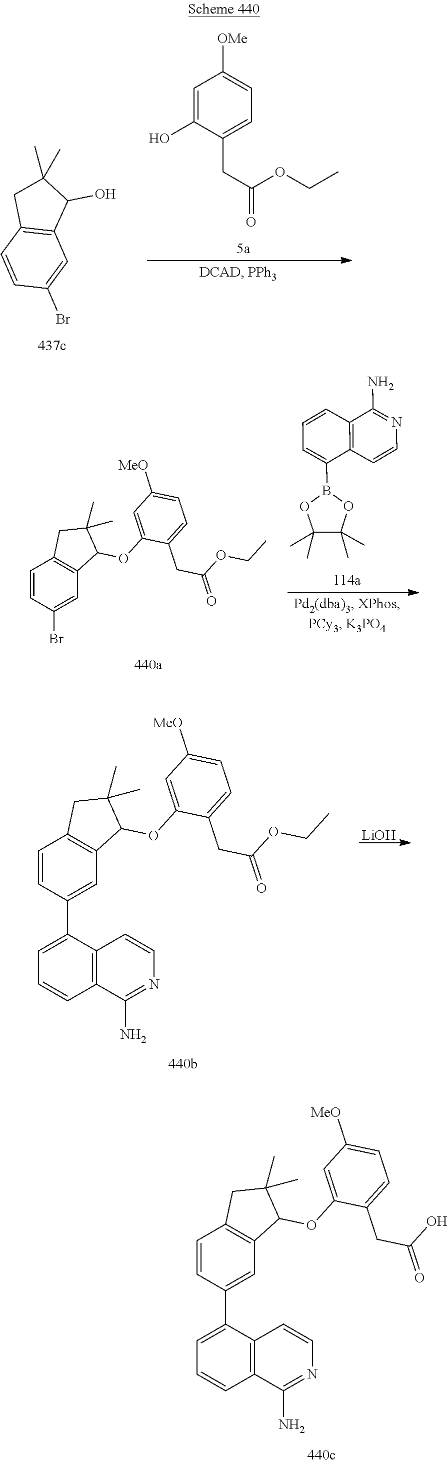

### Preparation of 2-(2-((6-(1-aminoisoquinolin-5-yl)-2,2-dimethyl-2,3-dihydro-1H-inden-1-yl)oxy)-4-methoxyphenyl)acetic acid (440c)

### Step-1: Preparation of ethyl 2-(2-((6-bromo-2,2-dimethyl-2,3-dihydro-1H-inden-1-yl)oxy)-4-methoxyphenyl)acetate (440a)

Compound 440a was prepared according to the procedure reported in step-2 of scheme 2, from 6-bromo-2,2-dimethyl-2,3-dihydro-1H-inden-1-ol (437c) (1 g, 4.15 mmol) in DCM (60 mL) using ethyl 2-(2-hydroxy-4-methoxyphenyl)acetate (5a) (0.872 g, 4.15 mmol), PPh₃ (1.088 g, 4.15 mmol) and a solution of DCAD (1.523 g, 4.15 mmol) in DCM (20 mL) to afford after workup and purification using method-P, ethyl 2-(2-((6-bromo-2,2-dimethyl-2,3-dihydro-1H-inden-1-yl)oxy)-4-methoxyphenyl)acetate (440a) (1.2 g, 67% yield) as a clear oil; MS (ES+): 433.1 and 435.1 (M+1); (ES−): 431.2 and 433.1 (M−1).

### Step-2: Preparation of ethyl 2-(2-((6-(1-aminoisoquinolin-5-yl)-2,2-dimethyl-2,3-dihydro-1H-inden-1-yl)oxy)-4-methoxyphenyl)acetate (440b)

Compound 440b was prepared according to the procedure reported in step-5 of scheme 1, from ethyl 2-(2-((6-bromo-2,2-dimethyl-2,3-dihydro-1H-inden-1-yl)oxy)-4-methoxyphenyl)acetate (440a) (200 mg, 0.462 mmol) in dioxane/2Me-THF (2 mL) using 5-(4,4,5,5-tetramethyl-1,3,2-dioxaborolan-2-yl)isoquinolin-1-amine (114a) (249 mg, 0.923 mmol), K₃PO₄ (3M aqueous solution, 308 μl, 0.923 mmol), PCy₃ (51.8 mg, 0.185 mmol). Pd₂(dba)₃ (85 mg, 0.092 mmol) and XPhos (44.0 mg, 0.092 mmol) to afford after workup and purification using method-U, ethyl 2-(2-((6-(1-aminoisoquinolin-5-yl)-2,2-dimethyl-2,3-dihydro-1H-inden-1-yl)oxy)-4-methoxyphenyl)acetate (440b) (52 mg, 23% yield) as a clear oil; MS (ES+): 497.2 (M+1); (ES−): 495.2 (M−1).

### Step-3: Preparation of 2-(2-((6-(1-aminoisoquinolin-5-yl)-2,2-dimethyl-2,3-dihydro-1H-inden-1-yl)oxy)-4-methoxyphenyl)acetic acid (440c)

Compound 440c was prepared according to the procedure reported in step-2 of scheme 1, from ethyl 2-(2-((6-(1-aminoisoquinolin-5-yl)-2,2-dimethyl-2,3-dihydro-1H-inden-1-yl)oxy)-4-methoxyphenyl)acetate (440b) (52 mg, 0.105 mmol) in THF (3 mL) using lithium hydroxide (33.2 mg, 1.385 mmol) in water (1 mL) to afford after workup and purification method-M, 2-(2-((6-(1-aminoisoquinolin-5-yl)-2,2-dimethyl-2,3-dihydro-1H-inden-1-yl)oxy)-4-methoxyphenyl)acetic acid (440c) (24 mg, 11% yield) HCl salt as a white solid; ¹H NMR (300 MHz, DMSO-d₆) δ 13.22 (s, 1H, D₂O exchangeable), 11.97 (s, 1H, D₂O exchangeable), 9.11 (s, 2H, D₂O exchangeable), 8.56 (dd, J=7.8, 1.8 Hz, 1H), 7.88-7.73 (m, 2H), 7.55 (d, J=7.3 Hz, 1H), 7.45 (d, J=7.7 Hz, 1H), 7.31 (dd, J=7.6, 1.7 Hz, 1H), 7.12 (d, J=1.7 Hz, 1H), 7.07 (d, J=8.3 Hz, 1H), 6.90-6.74 (m, 2H), 6.46 (dd, J=8.3, 2.3 Hz, 1H), 5.57 (s, 1H), 3.70 (s, 3H), 3.38-3.34 (m, 2H), 2.98-2.78 (m, 2H), 1.24 (s, 3H), 1.12 (s, 3H); MS (ES+): 469.2 (M+1); (ES−): 467.2 (M−1).

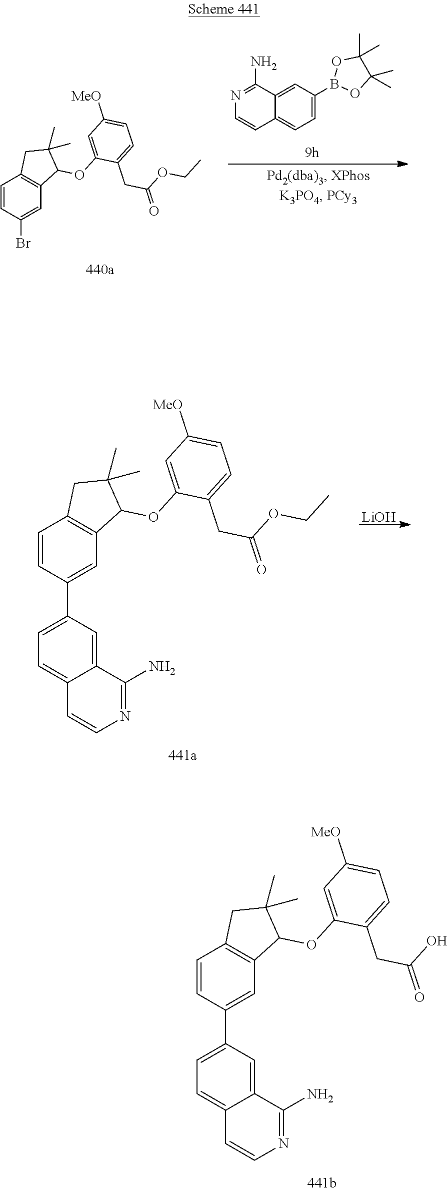

### Preparation of 2-(2-((6-(1-aminoisoquinolin-7-yl)-2,2-dimethyl-2,3-dihydro-1H-inden-1-yl)oxy)-4-methoxyphenyl)acetic acid (441b)

### Step-1: Preparation of ethyl 2-(2-((6-(1-aminoisoquinolin-7-yl)-2,2-dimethyl-2,3-dihydro-1H-inden-1-yl)oxy)-4-methoxyphenyl)acetate (441a)

Compound 441a was prepared according to the procedure reported in step-5 of scheme 1, from ethyl 2-(2-((6-bromo-2,2-dimethyl-2,3-dihydro-1H-inden-1-yl)oxy)-4-methoxyphenyl)acetate (440a) (150 mg, 0.346 mmol) in dioxane/2Me-THF (2 mL) using 7-(4,4,5,5-tetramethyl-1,3,2-dioxaborolan-2-yl)isoquinolin-1-amine (9h) (187 mg, 0.692 mmol), K₃PO₄ (3M aqueous solution, 231 μl, 0.692 mmol), PCy₃ (38.8 mg, 0.138 mmol), Pd₂(dba)₃ (63.4 mg, 0.069 mmol) and XPhos (33.0 mg, 0.069 mmol) to afford after workup and purification using method-U, ethyl 2-(2-((6-(1-aminoisoquinolin-7-yl)-2,2-dimethyl-2,3-dihydro-1H-inden-1-yl)oxy)-4-methoxyphenyl)acetate (441a) (86 mg, 50% yield) as a clear oil; MS (ES+): 497.3 (M+1); (ES−): 495.2 (M−1).

### Step-2: Preparation of 2-(2-((6-(1-aminoisoquinolin-7-yl)-2,2-dimethyl-2,3-dihydro-1H-inden-1-yl)oxy)-4-methoxyphenyl)acetic acid (441b)

Compound 441b was prepared according to the procedure reported in step-2 of scheme 1, from ethyl 2-(2-((6-(1-aminoisoquinolin-7-yl)-2,2-dimethyl-2,3-dihydro-1H-inden-1-yl)oxy)-4-methoxyphenyl)acetate (441a) (86 mg, 0.173 mmol) in THF (3 mL) using lithium hydroxide (24.87 mg, 1.038 mmol) in water (1 mL) to afford after workup and purification method-M, 2-(2-((6-(1-aminoisoquinolin-7-yl)-2,2-dimethyl-2,3-dihydro-1H-inden-1-yl)oxy)-4-methoxyphenyl)acetic acid (441b) (27 mg, 17% yield) HCl salt as a white solid; ¹H NMR (300 MHz, DMSO-d₆) δ 13.21 (s, 1H, D₂O exchangeable), 12.03 (s, 1H, D₂O) exchangeable), 9.08 (s, 2H, D₂O exchangeable), 8.81 (s, 1H), 8.15 (dd, J=8.5, 1.6 Hz, 1H), 8.00 (d, J=8.5 Hz, 1H), 7.77 (dd, J=7.8, 1.8 Hz, 1H), 7.67 (d, J=6.9 Hz, 1H), 7.57 (d, J=1.8 Hz, 1H), 7.45 (d, J=7.8 Hz, 1H), 7.24 (d, J=6.9 Hz, 1H), 7.10 (d, J=8.3 Hz, 1H), 6.85 (d, J=2.4 Hz, 1H), 6.49 (dd, J=8.3, 2.3 Hz, 1H), 5.61 (s, 1H), 3.75 (s, 3H), 3.40 (s, 2H), 2.93-2.76 (m, 2H), 1.24 (s, 3H), 1.08 (s, 3H); MS (ES+): 469.2 (M+1); (ES−): 467.3 (M−1).

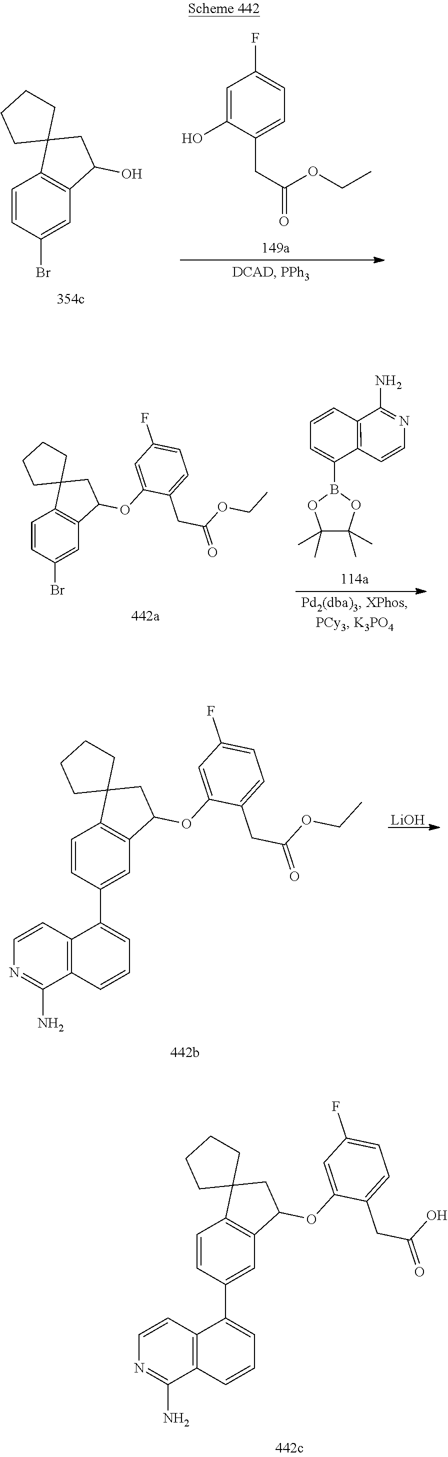

### Preparation of 2-(2-((5′-(1-aminoisoquinolin-5-yl)-2′,3′-dihydrospiro[cyclopentane-1,1′-inden]-3′-yl)oxy)-4-fluorophenyl)acetic acid (442c)

### Step-1: Preparation of ethyl 2-(2-((5′-bromo-2′,3′-dihydrospiro[cyclopentane-1,1′-inden]-3′-yl)oxy)-4-fluorophenyl)acetate (442a)

Compound 442a was prepared according to the procedure reported in step-2 of scheme 2, from 5′-bromo-2′,3′-dihydrospiro[cyclopentane-1,1′-inden]-3′-ol (354c) (1 g, 3.74 mmol) in DCM (30 mL) using ethyl 2-(4-fluoro-2-hydroxyphenyl)acetate (149a) (0.742 g, 3.74 mmol). PPh₃ (0.982 g, 3.74 mmol) and a solution of DCAD (1.374 g, 3.74 mmol) in DCM (10 mL) to afford after workup and purification using method-P, ethyl 2-(2-((5′-bromo-2′,3′-dihydrospiro[cyclopentane-1,1′-inden]-3′-yl)oxy)-4-fluorophenyl)acetate (442a) (1 g, 60% yield) as a clear oil; MS (ES+): 447.1 and 449.1 (M+1); (ES−): 445.0 and 447.0 (M−1).

### Step-2: Preparation of ethyl 2-(2-((5′-(1-aminoisoquinolin-5-yl)-2′,3′-dihydrospiro[cyclopentane-1,1′-inden]-3′-yl)oxy)-4-fluorophenyl)acetate (442b)

Compound 442b was prepared according to the procedure reported in step-5 of scheme 1, from ethyl 2-(2-((5′-bromo-2′,3′-dihydrospiro[cyclopentane-1,1′-inden]-3′-yl)oxy)-4-fluorophenyl)acetate (442a) (160 mg, 0.358 mmol) in dioxane/2Me-THF (2 mL) using 5-(4,4,5,5-tetramethyl-1,3,2-dioxaborolan-2-yl)isoquinolin-1-amine (114a) (193 mg, 0.715 mmol). K₃PO₄ (3M aqueous solution, 238 μl, 0.715 mmol), PCy₃ (40.1 mg, 0.143 mmol), Pd₂(dba)₃ (65.5 mg, 0.072 mmol) and XPhos (34.1 mg, 0.072 mmol) to afford after workup and purification using method-U, ethyl 2-(2-((5′-(1-aminoisoquinolin-5-yl)-2′,3′-dihydrospiro[cyclopentane-1,1′-inden]-3′-yl)oxy)-4-fluorophenyl)acetate (442b) (98 mg, 0.192 mmol, 53.7% yield) as a clear oil; MS (ES+): 511.3 (M+1); (ES−): 509.2 (M−1).

### Step-3: Preparation of 2-(2-((5′-(1-aminoisoquinolin-5-yl)-2′,3′-dihydrospiro[cyclopentane-1,1′-inden]-3′-yl)oxy)-4-fluorophenyl)acetic acid (442c)

Compound 442c was prepared according to the procedure reported in step-2 of scheme 1, from ethyl 2-(2-((5′-(1-aminoisoquinolin-5-yl)-2′,3′-dihydrospiro[cyclopentane-1,1′-inden]-3′-yl)oxy)-4-fluorophenyl)acetate (442b) (98 mg, 0.192 mmol) in THF (3 mL) using lithium hydroxide (25.7 mg, 1.073 mmol) in water (1 mL) to afford after workup and purification method-M, 2-(2-((5′-(1-aminoisoquinolin-5-yl)-2′,3′-dihydrospiro[cyclopentane-1,1′-inden]-3′-yl)oxy)-4-fluorophenyl)acetic acid (442c) (67 mg, 39% yield) HCl salt as a white solid; ¹H NMR (300 MHz, DMSO-d₆) ¿ 13.47 (s, 1H, D₂O exchangeable), 12.07 (s, 1H, D₂O exchangeable), 9.17 (s, 2H, D₂O exchangeable), 8.62 (d, J=8.2 Hz, 1H), 7.90 (dd. J=7.4, 1.2 Hz, 1H), 7.86-7.76 (m, 1H), 7.62 (d, J=7.2 Hz, 1H), 7.49 (d, J=7.9 Hz, 1H), 7.43 (dd, J=7.8, 1.7 Hz, 1H), 7.37 (d, J=1.6 Hz, 1H), 7.26-7.10 (m, 2H), 6.97 (d, J=7.2 Hz, 1H), 6.72 (td, J=8.5, 2.5 Hz, 1H), 5.97-5.88 (m, 1H), 3.43 (s, 2H), 2.73-2.62 (m, 1H), 1.99-1.94 (m, 1H), 1.94-1.67 (m, 8H); ¹⁹F NMR (282 MHz, DMSO-d₆) δ −112.90; MS (ES+): 483.2 (M+1); (ES−): 481.3 (M−1).

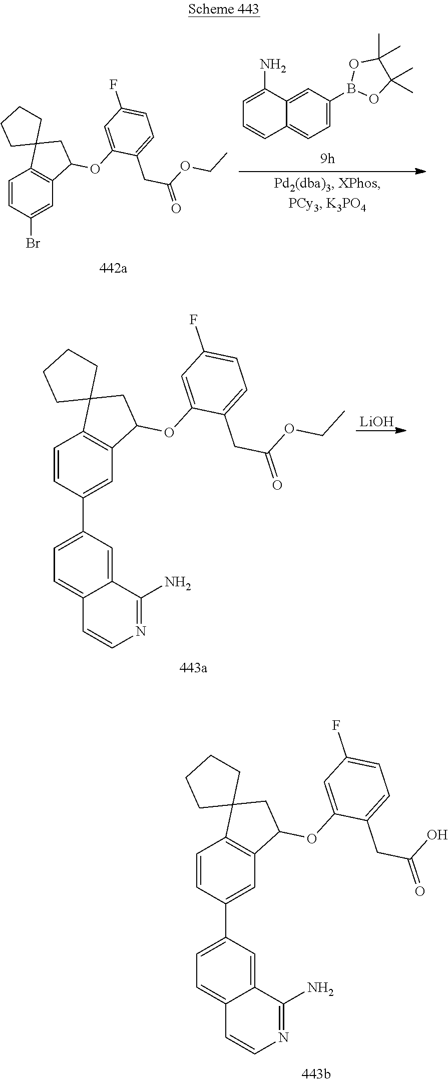

### Preparation of 2-(2-((5′-(1-aminoisoquinolin-7-yl)-2′,3′-dihydrospiro[cyclopentane-1,1′-inden]-3′-yl)oxy)-4-fluorophenyl)acetic acid (443b)

### Step-1: Preparation of ethyl 2-(2-((5′-(1-aminoisoquinolin-7-yl)-2′,3′-dihydrospiro[cyclopentane-1,1′-inden]-3′-yl)oxy)-4-fluorophenyl)acetate (443a)

Compound 443a was prepared according to the procedure reported in step-5 of scheme 1, from ethyl 2-(2-((5′-bromo-2′,3′-dihydrospiro[cyclopentane-1,1′-inden]-3′-yl)oxy)-4-fluorophenyl)acetate (442a) (140 mg, 0.313 mmol) in dioxane/2Me-THF (2 mL) using 7-(4,4,5,5-tetramethyl-1,3,2-dioxaborolan-2-yl)isoquinolin-1-amine (9h) (169 mg, 0.626 mmol), K₃PO₄ (3M aqueous solution, 209 μl, 0.626 mmol), PCy₃ (35.1 mg, 0.125 mmol), Pd₂(dba)₃ (57.3 mg, 0.063 mmol), XPhos (29.8 mg, 0.063 mmol) to afford after workup and purification using method-U, ethyl 2-(2-((5′-(1-aminoisoquinolin-7-yl)-2′3′-dihydrospiro[cyclopentane-1,1′-inden]-3′-yl)oxy)-4-fluorophenyl)acetate (443a) (62 mg, 39% yield) as a clear oil; MS (ES+): 511.3 (M+1); (ES−): 509.3 (M−1).

### Step-2: Preparation of 2-(2-((5′-(1-aminoisoquinolin-7-yl)-2′,3′-dihydrospiro[cyclopentane-1,1′-inden]-3′-yl)oxy)-4-fluorophenyl)acetic acid (443b)

Compound 443b was prepared according to the procedure reported in step-2 of scheme 1, from ethyl 2-(2-((5′-(1-aminoisoquinolin-7-yl)-2′,3′-dihydrospiro[cyclopentane-1,1′-inden]-3′-yl)oxy)-4-fluorophenyl)acetate (443a) (62 mg, 0.121 mmol) in THF (3 mL) using lithium hydroxide (22.48 mg, 0.939 mmol) in water (1 mL) to afford after workup and purification method-M, 2-(2-((5′-(1-aminoisoquinolin-7-yl)-2′,3′-dihydrospiro[cyclopentane-1,1′-inden]-3′-yl)oxy)-4-fluorophenyl)acetic acid (443b) (23 mg, 15% yield) HCl salt as a white solid; 1H NMR (300 MHz, DMSO-d₆) δ 13.22 (s, 1H, D₂O exchangeable), 12.18 (s, 1H, D₂O exchangeable), 9.06 (s, 2H, D₂O exchangeable), 8.88 (d, J=1.7 Hz, 1H), 8.30 (dd, J=8.5, 1.6 Hz, 1H), 8.03 (d, J=8.5 Hz, 1H), 7.90 (dd, J=8.0, 1.8 Hz, 1H), 7.83 (d, J=1.7 Hz, 1H), 7.70 (d, J=6.9 Hz, 1H), 7.49 (d, J=8.0 Hz, 1H), 7.30-7.12 (m, 3H), 6.75 (td, J=8.5, 2.4 Hz, 1H), 5.98-5.87 (m, 1H), 3.45 (d, J=3.8 Hz, 2H), 2.69-2.59 (m, 1H), 1.98-1.93 (m, 1H), 1.92-1.67 (m, 8H); ¹⁹F NMR (282 MHZ, DMSO-d₆) δ −112.84; MS (ES+): 483.2 (M+1); (ES−): 481.1 (M−1).

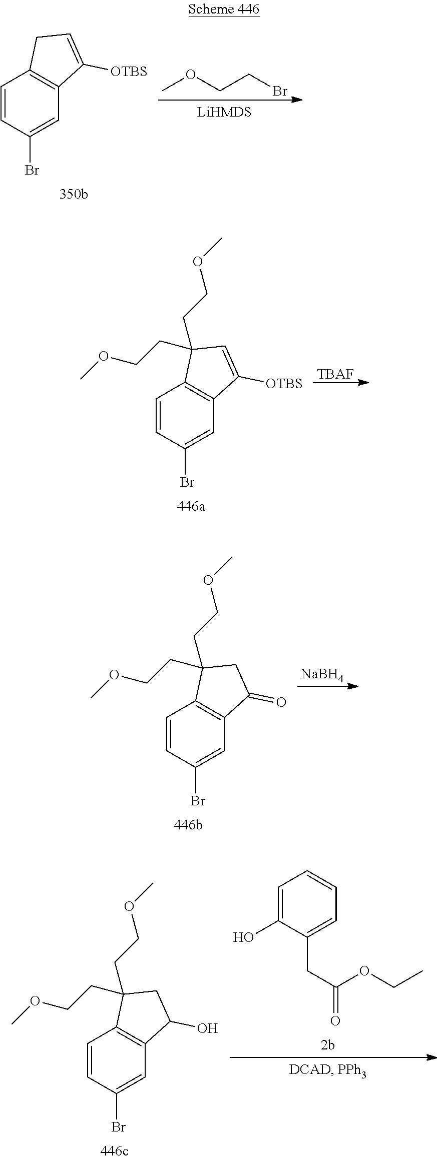

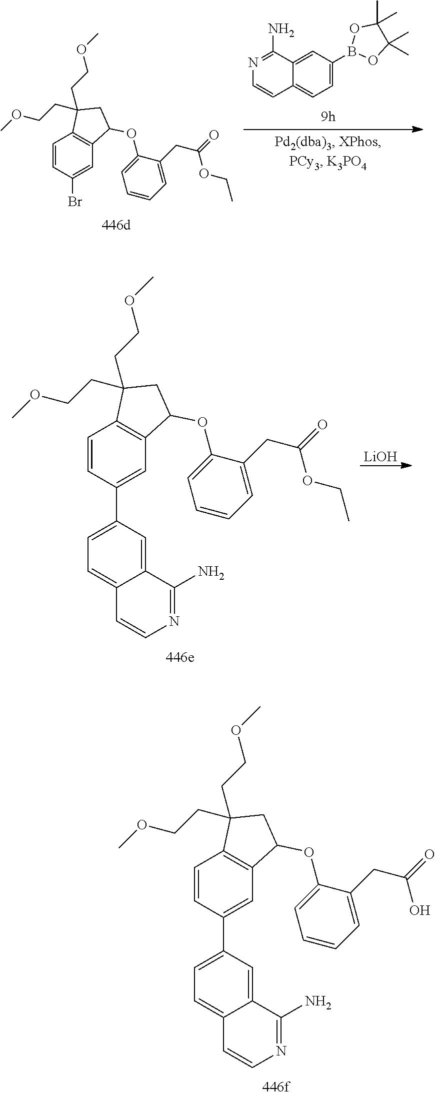

### Preparation of 2-(2-((6-(1-aminoisoquinolin-7-yl)-3,3-bis(2-methoxyethyl)-2,3-dihydro-1H-inden-1-yl)oxy)phenyl)acetic acid (446f)

### Step-1: Preparation of ((5-bromo-1,1-bis(2-methoxyethyl)-1H-inden-3-yl)oxy)(tert-butyl)dimethylsilane (446a)

Compound 446a was prepared according to the procedure reported in step-2 of scheme 350, from ((5-bromo-1H-inden-3-yl)oxy)(tert-butyl)dimethylsilane (350b) (6 g, 18.44 mmol) in anhydrous THF (10 mL), using LiHMDS (1.0 M in THF) (46.1 mL, 46.1 mmol), a solution of 1-bromo-2-methoxyethane (5.64 g, 40.6 mmol) in anhydrous THF (10 mL) stirring at −78° C. for 3 h to afford after work up and purification using method-V, ((5-bromo-1,1-bis(2-methoxyethyl)-1H-inden-3-yl)oxy)(tert-butyl)dimethylsilane (446a) (3.5 g, 43% yield) as a colorless oil; ¹H NMR (300 MHz, DMSO-d₆) δ 7.18-7.10 (m, 2H), 7.00 (t, J=1.1 Hz, 1H), 5.20 (s, 1H), 2.87-2.74 (m, 8H), 2.64-2.51 (m, 2H), 1.89-1.64 (m, 4H), 0.75 (s, 9H), 0.00 (s, 6H).

### Step-2: Preparation of 6-bromo-3,3-bis(2-methoxyethyl)-2,3-dihydro-1H-inden-1-one (446b)

Compound 446b was prepared according to the procedure reported in step-3 of scheme 350, from ((5-bromo-1,1-bis(2-methoxyethyl)-1H-inden-3-yl)oxy)(tert-butyl)dimethylsilane (446a) (3.5 g, 7.93 mmol) in anhydrous THF (100 mL) using TBAF (1.0 M in THF; 11.89 mL, 11.89 mmol) to afford after work up and purification using method-O, 6-bromo-3,3-bis(2-methoxyethyl)-2,3-dihydro-1H-inden-1-one (446b) (2.01 g, 77% yield) as a light brown oil; MS (ES+): 327.0 and 329.0 (M+1).

### Step-3: Preparation of 6-bromo-3,3-bis(2-methoxyethyl)-2,3-dihydro-1H-inden-1-ol (446c)

Compound 446c was prepared according to the procedure reported in step-1 of scheme 205, from 6-bromo-3,3-bis(2-methoxyethyl)-2,3-dihydro-1H-inden-1-one (446b) (2 g, 6.11 mmol) in anhydrous MeOH (20 mL) using sodium borohydride (0.347 g, 9.17 mmol) to afford after work up and purification using method-I, 6-bromo-3,3-bis(2-methoxyethyl)-2,3-dihydro-1H-inden-1-ol (446c) (1.5 g, 75% yield) as a clear oil; ¹H NMR (300 MHz, DMSO-d₆) δ 7.49-7.34 (m, 2H), 7.16 (d, J=8.0 Hz, 1H), 5.37 (d, J=5.8 Hz, 1H), 5.05 (q, J=6.8 Hz, 1H), 3.38-3.29 (m, 1H), 3.26-3.20 (m, 1H), 3.18 (s, 3H), 3.16-3.09 (m, 5H), 2.37-2.26 (m, 1H), 2.05-1.99 (m, 1H), 1.88-1.68 (m, 4H).

### Step-4: Preparation of ethyl 2-(2-((6-bromo-3,3-bis(2-methoxyethyl)-2,3-dihydro-1H-inden-1-yl)oxy)phenyl)acetate (446d)

Compound 446d was prepared according to the procedure reported in step-2 of scheme 2, from 6-bromo-3,3-bis(2-methoxyethyl)-2,3-dihydro-1H-inden-1-ol (446c) (1.5 g, 4.56 mmol) in DCM (30 mL) using ethyl 2-(2-hydroxyphenyl)acetate (2b) (0.821 g, 4.56 mmol), PPh₃ (1.195 g, 4.56 mmol) and a solution of DCAD (1.673 g, 4.56 mmol) in DCM (60 mL) to afford after workup and purification using method-V, ethyl 2-(2-((6-bromo-3,3-bis(2-methoxyethyl)-2,3-dihydro-1H-inden-1-yl)oxy)phenyl)acetate (446d) (1.01 g, 45% yield) as a clear oil; ¹H NMR (300 MHz, DMSO-d₆) δ 7.55 (dd, J=8.1, 2.0 Hz, 1H), 7.43 (d, J=1.9 Hz, 1H), 7.34-7.25 (m, 2H), 7.23 (dd, J=7.5, 1.7 Hz, 1H), 7.15 (d, J=8.1 Hz, 1H), 6.93 (td, J=7.4, 1.1 Hz, 1H), 5.79 (t, J=6.2 Hz, 1H), 4.03-3.91 (m, 2H), 3.54 (s, 2H), 3.33-3.17 (m, 4H), 3.16 (s, 3H), 3.14 (s, 3H), 2.69 (dd. J=13.8, 7.3 Hz, 1H), 1.99-1.76 (m, 5H), 1.05 (t, J=7.1 Hz, 3H); MS (ES+) 513.1 and 515.1 (M+Na).

### Step-5: Preparation of ethyl 2-(2-((6-(1-aminoisoquinolin-7-yl)-3,3-bis(2-methoxyethyl)-2,3-dihydro-1H-inden-1-yl)oxy)phenyl)acetate (446e)

Compound 446e was prepared according to the procedure reported in step-5 of scheme 1, from ethyl 2-(2-((6-bromo-3,3-bis(2-methoxyethyl)-2,3-dihydro-1H-inden-1-yl)oxy)phenyl)acetate (446d) (200 mg, 0.407 mmol) in dioxane/2Me-THF (2 mL) using 7-(4,4,5,5-tetramethyl-1,3,2-dioxaborolan-2-yl)isoquinolin-1-amine (9h) (220 mg, 0.814 mmol), K₃PO₄ (3M aqueous solution, 271 μl, 0.814 mmol), PCy₃ (45.7 mg, 0.163 mmol), Pd₂(dba)₃ (74.5 mg, 0.081 mmol) and XPhos (38.8 mg, 0.081 mmol) to afford after workup and purification using method-U, ethyl 2-(2-((6-(1-aminoisoquinolin-7-yl)-3,3-bis(2-methoxyethyl)-2,3-dihydro-1H-inden-1-yl)oxy)phenyl)acetate (446e) (71 mg, 32% yield) as a clear oil; MS (ES+): 555.3 (M+1); (ES−): 553.2 (M−1).

### Step-6: Preparation of 2-(2-((6-(1-aminoisoquinolin-7-yl)-3,3-bis(2-methoxyethyl)-2,3-dihydro-1H-inden-1-yl)oxy)phenyl)acetic acid (446f)

Compound 446f was prepared according to the procedure reported in step-2 of scheme 1, from ethyl 2-(2-((6-(1-aminoisoquinolin-7-yl)-3,3-bis(2-methoxyethyl)-2,3-dihydro-1H-inden-1-yl)oxy)phenyl)acetate (446e) (71 mg, 0.128 mmol) in THF (3 mL) using lithium hydroxide (29.2 mg, 1.221 mmol) in water (1 mL) to afford after workup and purification method-M, 2-(2-((6-(1-aminoisoquinolin-7-yl)-3,3-bis(2-methoxyethyl)-2,3-dihydro-1H-inden-1-yl)oxy)phenyl)acetic acid (446f) (22 mg, 10% yield) HCl salt as a white solid; ¹H NMR (300 MHz, DMSO-d₆) δ 13.17 (s, 1H, D₂O exchangeable), 12.11 (s, 1H, D₂O exchangeable), 9.05 (s, 2H, D₂O exchangeable), 8.89 (s, 1H), 8.32 (dd, J=8.4, 1.6 Hz, 1H), 8.04 (d, J=8.5 Hz, 1H), 7.90 (dd, J=8.1, 1.8 Hz, 1H), 7.83 (d, J=1.8 Hz, 1H), 7.70 (d, J=6.9 Hz, 1H), 7.50 (d, J=8.0 Hz, 1H), 7.35-7.15 (m, 4H), 6.93 (t, J=7.3 Hz, 1H), 5.86 (t, J=6.2 Hz, 1H), 3.51 (d, J=1.6 Hz, 2H), 3.30-3.20 (m, 4H), 3.18 (s, 3H), 3.16 (s, 3H), 2.81-2.71 (m, 1H), 2.10-1.84 (m, 5H); MS (ES+): 527.2 (M+1); (ES−): 525.2 (M−1).

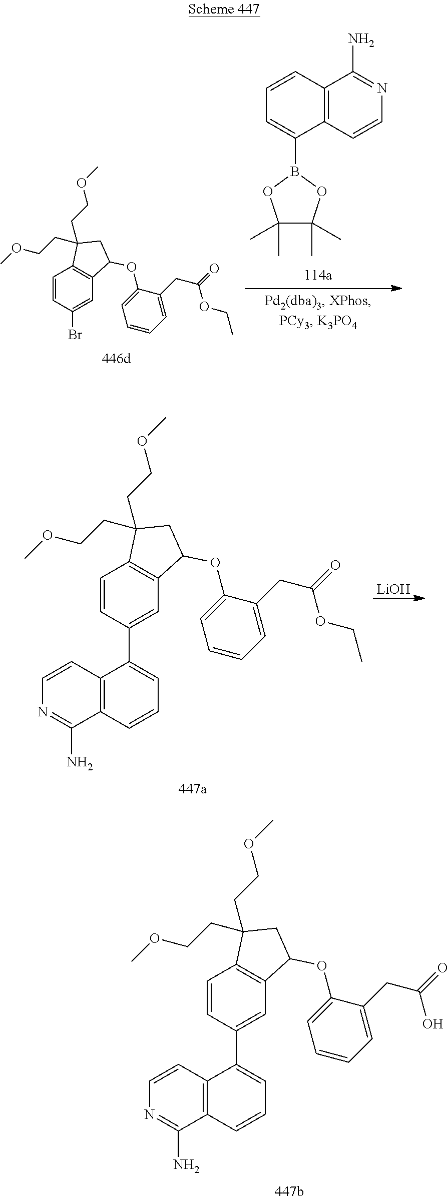

### Preparation of 2-(2-((6-(1-aminoisoquinolin-5-yl)-3,3-bis(2-methoxyethyl)-2,3-dihydro-1H-inden-1-yl)oxy)phenyl)acetic acid (447b)

### Step-1: Preparation of ethyl 2-(2-((6-(1-aminoisoquinolin-5-yl)-3,3-bis(2-methoxyethyl)-2,3-dihydro-1H-inden-1-yl)oxy)phenyl)acetate (447a)

Compound 447a was prepared according to the procedure reported in step-5 of scheme 1, from ethyl 2-(2-((6-bromo-3,3-bis(2-methoxyethyl)-2,3-dihydro-1H-inden-1-yl)oxy)phenyl)acetate (446d) (200 mg, 0.407 mmol) in dioxane/2Me-THF (2 mL) using 5-(4,4,5,5-tetramethyl-1,3,2-dioxaborolan-2-yl)isoquinolin-1-amine (114a) (220 mg, 0.814 mmol), K₃PO₄ (3M aqueous solution, 271 μl, 0.814 mmol), PCy₃ (45.7 mg, 0.163 mmol), Pd₂(dba)₃ (74.5 mg, 0.081 mmol) and XPhos (38.8 mg, 0.081 mmol) to afford after workup and purification using method-U, ethyl 2-(2-((6-(1-aminoisoquinolin-5-yl)-3,3-bis(2-methoxyethyl)-2,3-dihydro-1H-inden-1-yl)oxy)phenyl)acetate (447a) (162 mg, 0.292 mmol, 71.8% yield) as a clear oil; MS (ES+): 555.3 (M+1); (ES−): 553.2 (M−1).

### Step-2: Preparation of 2-(2-((6-(1-aminoisoquinolin-5-yl)-3,3-bis(2-methoxyethyl)-2,3-dihydro-1H-inden-1-yl)oxy)phenyl)acetic acid (447b)

Compound 447b was prepared according to the procedure reported in step-2 of scheme 1, from ethyl 2-(2-((6-(1-aminoisoquinolin-5-yl)-3,3-bis(2-methoxyethyl)-2,3-dihydro-1H-inden-1-yl)oxy)phenyl)acetate (447a) (162 mg, 0.292 mmol) in THF (3 mL) using lithium hydroxide (29.2 mg, 1.221 mmol) in water (1 mL) to afford after workup and purification method-M, 2-(2-((6-(1-aminoisoquinolin-5-yl)-3,3-bis(2-methoxyethyl)-2,3-dihydro-1H-inden-1-yl)oxy)phenyl)acetic acid (447b) (112 mg, 52% yield) HCl salt as a white solid; ¹H NMR (300 MHz, DMSO-d₆) δ 13.55 (s, 1H, D₂O) exchangeable), 12.02 (s, 1H, D₂O exchangeable), 9.26 (s, 2H, D₂O exchangeable), 8.65 (d, J=8.3 Hz, 1H), 7.99-7.88 (m, 1H), 7.84 (t, J=7.8 Hz, 1H), 7.65 (d, J=7.3 Hz, 1H), 7.53-7.35 (m, 3H), 7.27 (td, J=7.7, 7.1, 1.7 Hz, 1H), 7.23-7.14 (m, 2H), 6.99 (d, J=7.2 Hz, 1H), 6.95-6.85 (m, 1H), 5.86 (t, J=6.2 Hz, 1H), 3.48 (d, J=2.7 Hz, 2H), 3.32-3.23 (m, 4H), 3.20 (s, 3H), 3.18 (s, 3H), 2.84-2.72 (m, 1H), 2.18-1.82 (m, 5H); MS (ES+): 527.3 (M+1); (ES−) 525.2 (M−1).

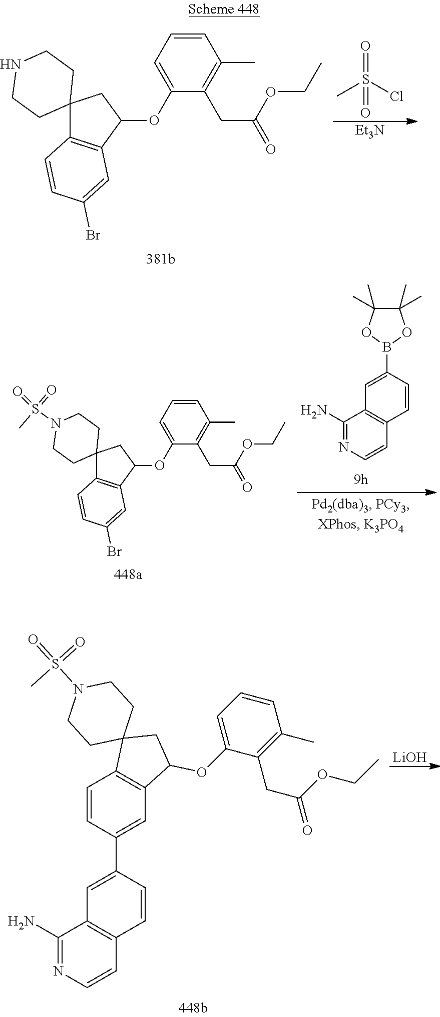

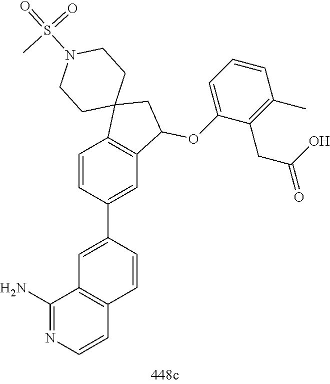 [SMILES: Cc1cccc(OC2CC3(CCN(S(C)(=O)=O)CC3)c3ccc(-c4ccc5ccnc(N)c5c4)cc32)c1CC(=O)O]

### Preparation of 2-(2-((5-(1-aminoisoquinolin-7-yl)-1′-(methylsulfonyl)-2,3-dihydrospiro[indene-1,4′-piperidin]-3-yl)oxy)-6-methylphenyl)acetic acid (448c)

### Step-1: Preparation of ethyl 2-(2-((5-bromo-1′-(methylsulfonyl)-2,3-dihydrospiro[indene-1,4′-piperidin]-3-yl)oxy)-6-methylphenyl)acetate (448a)

To an ice cold solution of ethyl 2-(2-((5-bromo-2,3-dihydrospiro[indene-1,4′-piperidin]-3-yl)oxy)-6-methylphenyl)acetate, TFA (381b) (0.7 g, 1.223 mmol) in DCM (10 mL) was added triethylamine (0.545 mL, 3.91 mmol) followed by methanesulfonyl chloride (0.182 g, 1.590 mmol) and was allowed to warm to RT stirring for 2 h. Reaction mixture was quenched with NH₄Cl solution (3 mL) and was extracted with CH₂Cl₂. Combined organics were washed with H₂O, brine, dried, filtered and concentrated under vacuum. The residue obtained was purified using method-O to afford ethyl 2-(2-((5-bromo-1′-(methylsulfonyl)-2,3-dihydrospiro[indene-1,4′-piperidin]-3-yl)oxy)-6-methylphenyl)acetate (448a) (0.42 g, 64% yield) as clear gel; MS (ES+): 558.1 (M+Na).

### Step-2: Preparation of ethyl 2-(2-((5-(1-aminoisoquinolin-7-yl)-1′-(methylsulfonyl)-2,3-dihydrospiro[indene-1,4′-piperidin]-3-yl)oxy)-6-methylphenyl)acetate (448b)

Compound 448b was prepared according to the procedure reported in step-3 of scheme 112, from ethyl 2-(2-((5-bromo-1′-(methylsulfonyl)-2,3-dihydrospiro[indene-1,4′-piperidin]-3-yl)oxy)-6-methylphenyl)acetate (448a) (0.21 g, 0.391 mmol) in dioxane/2-MeTHF (3 mL) using 7-(4,4,5,5-tetramethyl-1,3,2-dioxaborolan-2-yl)isoquinolin-1-amine (9h) (0.211 g, 0.783 mmol), K₃PO₄ (3M aqueous solution, 0.261 mL, 0.783 mmol), PCy₃ (0.044 g, 0.157 mmol), XPhos (0.019 g, 0.039 mmol) and Pd₂(dba)₃ (0.072 g, 0.078 mmol) and heating at 100° C. for 4 h to afford after workup and purification method-F, ethyl 2-(2-((5-(1-aminoisoquinolin-7-yl)-1′-(methylsulfonyl)-2,3-dihydrospiro[indene-1,4′-piperidin]-3-yl)oxy)-6-methylphenyl)acetate (448b) (82 mg, 35% yield) as a pale yellow solid; MS (ES+): 600.2 (M+1).

### Step-3: Preparation of 2-(2-((5-(1-aminoisoquinolin-7-yl)-1′-(methylsulfonyl)-2,3-dihydrospiro[indene-1,4′-piperidin]-3-yl)oxy)-6-methylphenyl)acetic acid (448c)

Compound 448c was prepared according to the procedure reported in step-2 of scheme 1, from ethyl 2-(2-((5-(1-aminoisoquinolin-7-yl)-1′-(methylsulfonyl)-2,3-dihydrospiro[indene-1,4′-piperidin]-3-yl)oxy)-6-methylphenyl)acetate (448b) (80 mg, 0.133 mmol) in MeOH (1 mL) and THF (1 mL) using lithium hydroxide (1N solution, 534 μl, 0.534 mmol) to afford after work up and purification using method-G, 2-(2-((5-(1-aminoisoquinolin-7-yl)-1′-(methylsulfonyl)-2,3-dihydrospiro[indene-1,4′-piperidin]-3-yl)oxy)-6-methylphenyl)acetic acid (448c) (23 mg, 30% yield) HCl salt as a white solid; ¹H NMR (300 MHz, DMSO-d₆) δ 13.17 (s, 1H, D₂O exchangeable), 12.11 (s, 1H, D₂O exchangeable), 9.09 (s, 2H, DO exchangeable), 8.90 (s, 1H), 8.32 (dd, J=8.5, 1.6 Hz, 1H), 8.04 (d, J=8.5 Hz, 1H), 7.98-7.82 (m, 2H), 7.69 (d, J=6.9 Hz, 1H), 7.63 (d, J=8.0 Hz, 1H), 7.33-7.11 (m, 3H), 6.90-6.77 (m, 1H), 5.94 (dd, J=6.8, 3.7 Hz, 1H), 3.70-3.45 (m, 4H), 3.05-2.75 (m, 5H), 2.65 (dd, J=13.9, 6.8 Hz, 1H), 2.22 (s, 3H), 2.13-1.93 (m, 3H), 1.82-1.60 (m, 2H); MS (ES+): 572.2 (M+1); (ES−): 570.2 (M−1).

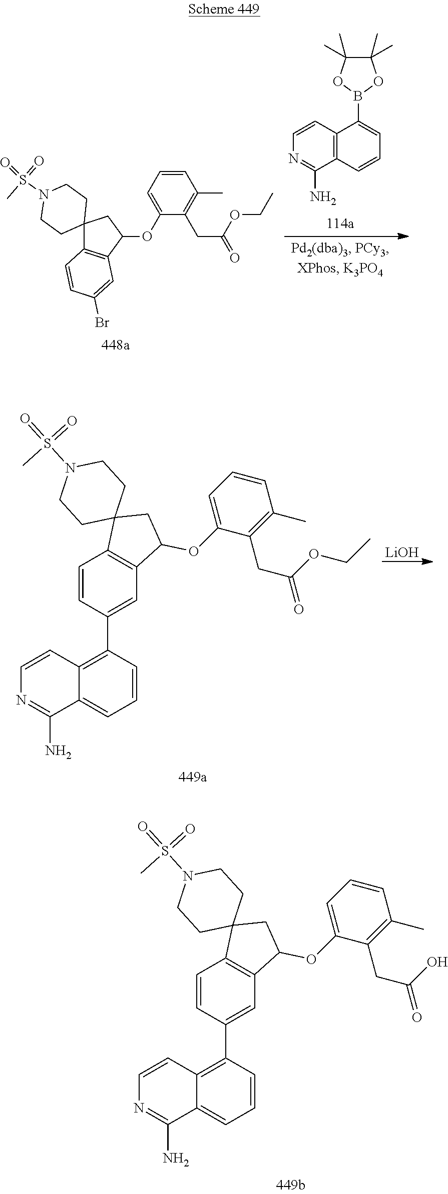

### Preparation of 2-(2-((5-(1-aminoisoquinolin-5-yl)-1′-(methylsulfonyl)-2,3-dihydrospiro[indene-1,4′-piperidin]-3-yl)oxy)-6-methylphenyl)acetic acid (449b)

### Step-1: Preparation of ethyl 2-(2-((5-(1-aminoisoquinolin-5-yl)-1′-(methylsulfonyl)-2,3-dihydrospiro[indene-1,4′-piperidin]-3-yl)oxy)-6-methylphenyl)acetate (449a)

Compound 449a was prepared according to the procedure reported in step-5 of scheme 1, from ethyl 2-(2-((5-bromo-1′-(methylsulfonyl)-2,3-dihydrospiro[indene-1,4′-piperidin]-3-yl)oxy)-6-methylphenyl)acetate (448a) (210 mg, 0.391 mmol) in dioxane/2Me-THF (3 mL) using 5-(4,4,5,5-tetramethyl-1,3,2-dioxaborolan-2-yl)isoquinolin-1-amine (114a) (211 mg, 0.783 mmol), K₃PO₄ (3M aqueous solution, 261 μl, 0.783 mmol). PCy₃ (43.9 mg, 0.157 mmol), Pd₂(dba)₃ (71.7 mg, 0.078 mmol) and XPhos (18.66 mg, 0.039 mmol) to afford after workup and purification using method-F, ethyl 2-(2-((5-(1-aminoisoquinolin-5-yl)-1′-(methylsulfonyl)-2,3-dihydrospiro[indene-1,4′-piperidin]-3-yl)oxy)-6-methylphenyl)acetate (449a) (123 mg, 0.205 mmol, 52.4% yield) as a pale yellow solid; MS (ES+): 600.2 (M+1).

### Step-2: Preparation of 2-(2-((5-(1-aminoisoquinolin-5-yl)-1′-(methylsulfonyl)-2,3-dihydrospiro[indene-1,4′-piperidin]-3-yl)oxy)-6-methylphenyl)acetic acid (449b)

Compound 449b was prepared according to the procedure reported in step-2 of scheme 1, from ethyl 2-(2-((5-(1-aminoisoquinolin-5-yl)-1′-(methylsulfonyl)-2,3-dihydrospiro[indene-1,4′-piperidin]-3-yl)oxy)-6-methylphenyl)acetate (449a) (120 mg, 0.2 mmol) in MeOH (1.6 mL) and THF (1.6 mL) using lithium hydroxide (1N aqueous solution; 800 μl, 0.8 mmol) stirring for 28 h to afford after workup and purification using method-G, 2-(2-((5-(1-aminoisoquinolin-5-yl)-1′-(methylsulfonyl)-2,3-dihydrospiro[indene-1,4′-piperidin]-3-yloxy)-6-methylphenyl)acetic acid (449b) (45 mg, 39% yield) HCl salt as a white solid; 1H NMR (300 MHz, DMSO-d₆) δ 13.43 (s, 1H, D₂O exchangeable), 12.06 (s, 1H, D₂O exchangeable), 9.21 (s, 2H, DO exchangeable), 8.63 (d, J=8.2 Hz, 1H), 7.97-7.77 (m, 2H), 7.69-7.55 (m, 2H), 7.51-7.39 (m, 2H), 7.20-7.07 (m, 2H), 6.97 (d, J=7.3 Hz, 1H), 6.80 (dd, J=7.4, 1.5 Hz, 1H), 5.92 (dd, J=6.8, 4.1 Hz, 1H), 3.60 (t, J=12.1 Hz, 2H), 3.50 (s, 2H), 3.06-2.79 (m, 5H), 2.72 (dd, J=13.7, 6.9 Hz, 1H), 2.20 (s, 3H), 2.14-1.89 (m, 3H), 1.85-1.64 (m, 2H); MS (ES+): 572.2 (M+1); (ES−): 570.2 (M−1).

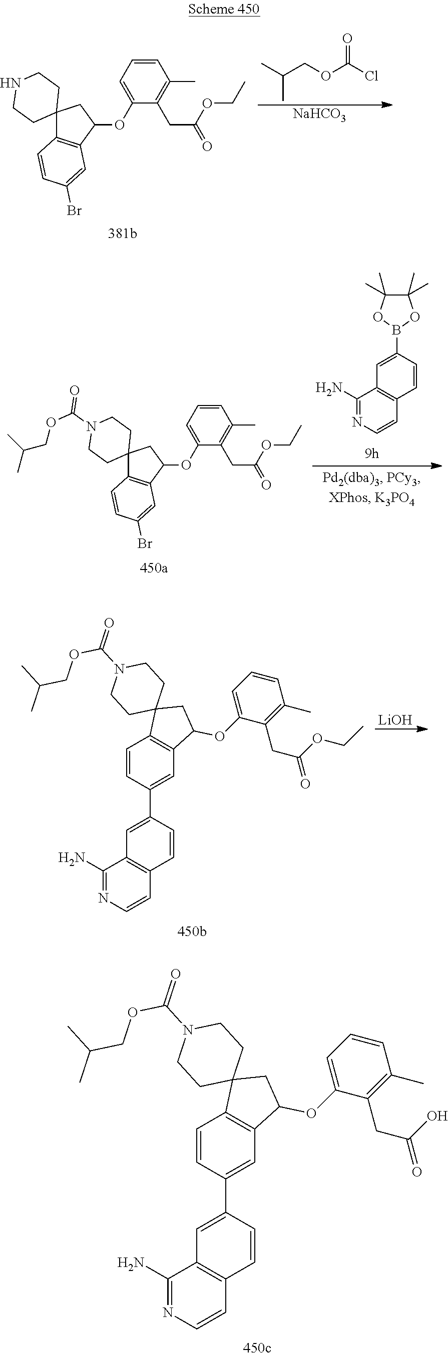

### Preparation of 2-(2-((5-(1-aminoisoquinolin-7-yl)-1′-(isobutoxycarbonyl)-2,3-dihydrospiro[indene-1,4′-piperidin]-3-yl)oxy)-6-methylphenyl)acetic acid (450c)

### Step-1: Preparation of isobutyl 5-bromo-3-(2-(2-ethoxy-2-oxoethyl)-3-methylphenoxy)-2,3-dihydrospiro[indene-1,4′-piperidine]-1′-carboxylate (450a)

Compound 450a was prepared according to the procedure reported in step-1 of scheme 370, from ethyl 2-(2-((5-bromo-2,3-dihydrospiro[indene-1,4′-piperidin]-3-yl)oxy)-6-methylphenyl)acetate, TFA (381b) (0.7 g, 1.223 mmol) in THF (10 mL) using sodium bicarbonate (3.06 mL, 6.11 mmol) and isobutyl carbonochloridate (0.251 g, 1.834 mmol) to afford after workup and purification using method-P, isobutyl 5-bromo-3-(2-(2-ethoxy-2-oxoethyl)-3-methylphenoxy)-2,3-dihydrospiro[indene-1,4′-piperidine]-1′-carboxylate (450a) (0.54 g, 79% yield) as a clear oil; MS (ES+): 580.3 (M+Na).

### Step-2: Preparation of isobutyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (450b)

Compound 450b was prepared according to the procedure reported in step-3 of scheme 112, from isobutyl 5-bromo-3-(2-(2-ethoxy-2-oxoethyl)-3-methylphenoxy)-2,3-dihydrospiro[indene-1,4′-piperidine]-1′-carboxylate (450a) (0.27 g, 0.483 mmol) in dioxane/2-MeTHF (4 mL) using 7-(4,4,5,5-tetramethyl-1,3,2-dioxaborolan-2-yl)isoquinolin-1-amine (9h) (0.261 g, 0.967 mmol), K₃PO₄ (3M aqueous solution, 0.322 mL, 0.967 mmol). PCy₃ (0.054 g, 0.193 mmol), XPhos (0.023 g, 0.048 mmol), Pd₂(dba)₃ (0.089 g, 0.097 mmol) and heating at 100° C. for 4 h to afford after workup and purification using method-F, isobutyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (450b) (142 mg, 47% yield) as a pale yellow solid; MS (ES+): 622.3 (M+1).

### Step-3: Preparation of 2-(2-((5-(1-aminoisoquinolin-7-yl)-1′-(isobutoxycarbonyl)-2,3-dihydrospiro[indene-1,4′-piperidin]-3-yl)oxy)-6-methylphenyl)acetic acid (450c)

Compound 450c was prepared according to the procedure reported in step-2 of scheme 1, from isobutyl 5-(1-aminoisoquinolin-7-yl)-3-(2-(2-ethoxy-2-oxoethyl)-3-methylphenoxy)-2,3-dihydrospiro[indene-1,4′-piperidine]-1′-carboxylate (450b) (140 mg, 0.225 mmol) in MeOH (1.8 mL) and THF (1.8 mL) using lithium hydroxide (1 N solution, 901 μl, 0.901 mmol) to afford after work up and purification using method-BV, 2-(2-((5-(1-aminoisoquinolin-7-yl)-1′-(isobutoxycarbonyl)-2,3-dihydrospiro[indene-1,4′-piperidin]-3-yl)oxy)-6-methylphenyl)acetic acid (450c) (28 mg, 21% yield) HCl salt as an off-white solid; ¹H NMR (300 MHz, DMSO-d₆) δ 13.13 (s, 1H, D₂O exchangeable), 12.05 (s, 1H, D₂O exchangeable), 9.00 (s, 2H, D₂O exchangeable), 8.87 (s, 1H), 8.30 (dd, J=8.7, 1.6 Hz, 1H), 8.03 (d, J=8.6 Hz, 1H), 7.93-7.82 (m, 2H), 7.69 (d, J=6.9 Hz, 1H), 7.57 (d, J=8.0 Hz, 1H), 7.28-7.11 (m, 3H), 6.86-6.79 (m, 1H), 5.97-5.87 (m, 1H), 4.14-3.92 (m, 2H), 3.81 (d, 2H), 3.61-3.42 (m, 2H), 3.21-2.90 (m, 2H), 2.77-2.62 (m, 1H), 2.22 (s, 3H), 2.17-2.03 (m, 1H), 1.99-1.73 (m, 3H), 1.68-1.48 (m, 2H), 0.91 (d, J=6.7 Hz, 6H); MS (ES+): 594.3 (M+1); (ES−): 592.2 (M−1).

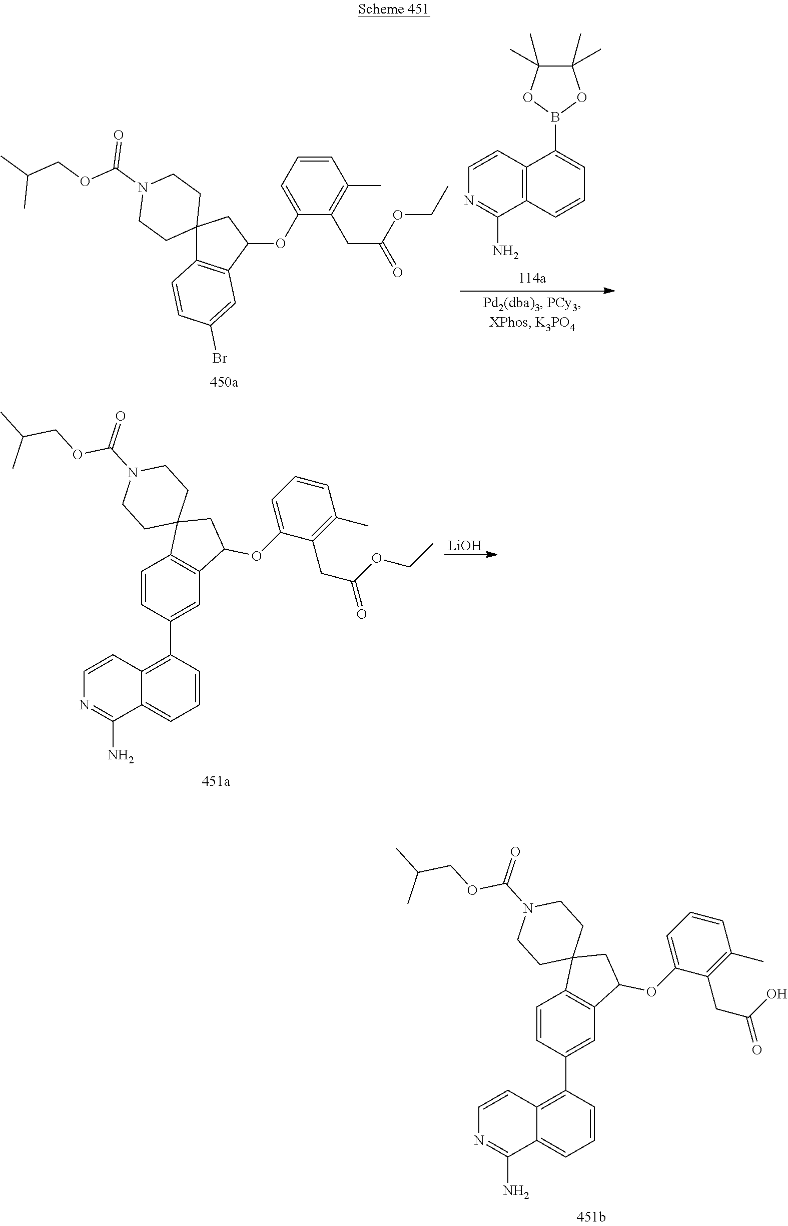

### Preparation of 2-(2-((5-(1-aminoisoquinolin-5-yl)-1′-(isobutoxycarbonyl)-2,3-dihydrospiro[indene-1,4′-piperidin]-3-yl)oxy)-6-methylphenyl)acetic acid (451b)

### Step-1: Preparation of isobutyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (451a)

Compound 451a was prepared according to the procedure reported in step-5 of scheme 1, from isobutyl 5-bromo-3-(2-(2-ethoxy-2-oxoethyl)-3-methylphenoxy)-2,3-dihydrospiro[indene-1,4′-piperidine]-1′-carboxylate (450a) (270 mg, 0.483 mmol) in dioxane/2Me-THF (3 mL) using 5-(4,4,5,5-tetramethyl-1,3,2-dioxaborolan-2-yl)isoquinolin-1-amine (114a) (261 mg, 0.967 mmol), K₃PO₄ (3M aqueous solution, 322 μl, 0.967 mmol), PCy₃ (54.2 mg, 0.193 mmol), Pd₂(dba)₃ (89 mg, 0.097 mmol) and XPhos (23.05 mg, 0.048 mmol) stirring at 100° C. for 4 h to afford after workup and purification using method-F, isobutyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (451a) as a pale yellow solid; MS (ES+): 622.3 (M+1).

### Step-2: Preparation of 2-(2-((5-(1-aminoisoquinolin-5-yl)-1′-(isobutoxycarbonyl)-2,3-dihydrospiro[indene-1,4′-piperidin]-3-yl)oxy)-6-methylphenyl)acetic acid (451b)

Compound 451b was prepared according to the procedure reported in step-2 of scheme 1, from isobutyl 5-(1-aminoisoquinolin-5-yl)-3-(2-(2-ethoxy-2-oxoethyl)-3-methylphenoxy)-2,3-dihydrospiro[indene-1,4′-piperidine]-1′-carboxylate (451a) (135 mg, 0.217 mmol) in MeOH (1.8 mL) and THF (1.8 mL) using lithium hydroxide (1N aqueous solution; 868 μl, 0.868 mmol) to afford after workup and purification method-G, 2-(2-((5-(1-aminoisoquinolin-5-yl)-1′-(isobutoxycarbonyl)-2,3-dihydrospiro[indene-1,4′-piperidin]-3-yl)oxy)-6-methylphenyl)acetic acid (451b) (67 mg, 52% yield) HCl salt as a white solid; 1H NMR (300 MHz, DMSO-d₆) δ 13.46 (s, 1H, D₂O exchangeable), 12.06 (s, 1H, D₂O exchangeable), 9.22 (s, 2H, D₂O exchangeable), 8.63 (d, J=8.2 Hz, 1H), 7.93-7.79 (m, 2H), 7.62 (d, J=7.3 Hz, 1H), 7.55 (d, J=7.9 Hz, 1H), 7.47-7.36 (m, 2H), 7.20-7.06 (m), 2H), 6.96 (d, J=7.2 Hz, 1H), 6.80 (dd, J=6.5, 2.1 Hz, 1H), 5.98-5.83 (m, 1H), 4.16-3.92 (m, 2H), 3.82 (d, J=6.5 Hz, 2H), 3.50 (s, 2H), 3.13-2.92 (m, 2H), 2.77 (dd, J=13.9, 6.9 Hz, 1H), 2.20 (s, 3H), 2.16-2.04 (m, 1H), 1.99-1.73 (m, 3H), 1.73-1.50 (m, 2H), 0.91 (d, J=6.7 Hz, 6H); MS (ES+): 594.3 (M+1); (ES−): 592.2 (M−1).

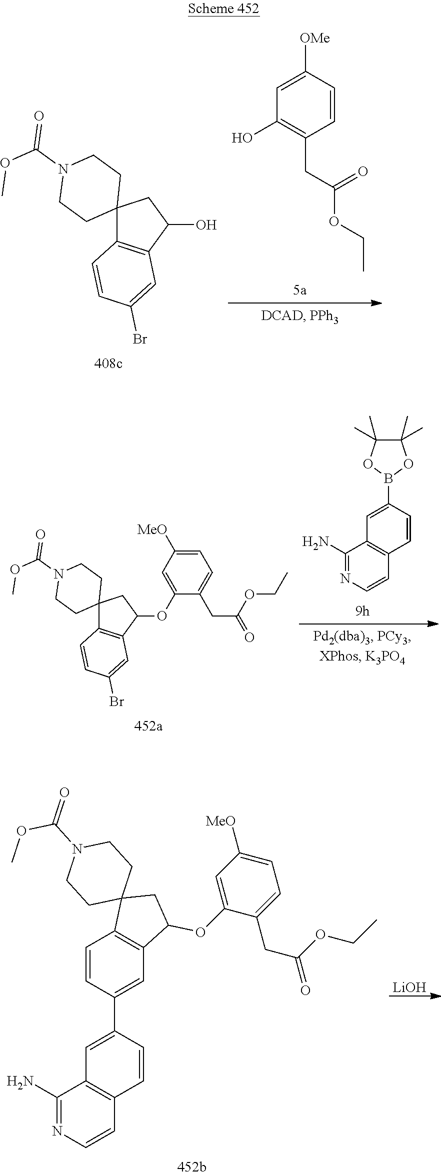

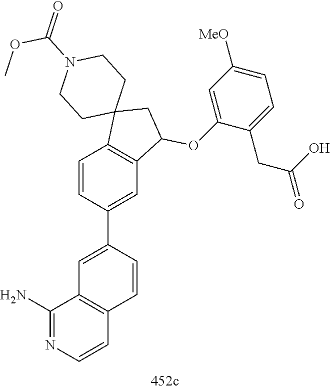 [SMILES: COC(=O)N1CCC2(CC1)CC(Oc1cc(OC)ccc1CC(=O)O)c1cc(-c3ccc4ccnc(N)c4c3)ccc12]

### Preparation of 2-(2-((5-(1-aminoisoquinolin-7-yl)-1′-(methoxycarbonyl)-2,3-dihydrospiro[indene-1,4′-piperidin]-3-yl)oxy)-4-methoxyphenyl)acetic acid (452c)

### Step-1: Preparation of methyl 5-bromo-3-(2-(2-ethoxy-2-oxoethyl)-5-methoxyphenoxy)-2,3-dihydrospiro[indene-1,4′-piperidine]-1′-carboxylate (452a)

Compound 452a was prepared according to the procedure reported in step-2 of scheme 2, from methyl 5-bromo-3-hydroxy-2,3-dihydrospiro[indene-1,4′-piperidine]-1′-carboxylate (408c) (0.6 g, 1.764 mmol) in DCM (30 mL) using PPh₃ (0.555 g, 2.116 mmol), ethyl 2-(2-hydroxy-4-methoxyphenyl)acetate (5a) (0.445 g, 2.116 mmol) and a solution of DCAD (0.777 g, 2.116 mmol) in DCM (10 mL) to afford after workup and purification using method-O, methyl 5-bromo-3-(2-(2-ethoxy-2-oxoethyl)-5-methoxyphenoxy)-2,3-dihydrospiro[indene-1,4′-piperidine]-1′-carboxylate (452a) (0.51 g, 0.958 mmol, 54.3% yield) as a white solid; MS (ES+): 554.1 (M+Na).

### Step-2: Preparation of methyl 5-(1-aminoisoquinolin-7-yl)-3-(2-(2-ethoxy-2-oxoethyl)-5-methoxyphenoxy)-2,3-dihydrospiro[indene-1,4′-piperidine]-1′-carboxylate (452b)

Compound 452b was prepared according to the procedure reported in step-3 of scheme 112, from methyl 5-bromo-3-(2-(2-ethoxy-2-oxoethyl)-5-methoxyphenoxy)-2,3-dihydrospiro[indene-1,4′-piperidine]-1′-carboxylate (452a) (0.25 g, 0.470 mmol) in dioxane/2-MeTHF (4 mL) using 7-(4,4,5,5-tetramethyl-1,3,2-dioxaborolan-2-yl)isoquinolin-1-amine (9h) (0.254 g, 0.939 mmol), K₃PO₄ (3M aqueous solution, 0.313 mL, 0.939 mmol). PCy₃ (0.040 g, 0.141 mmol), XPhos (0.022 g, 0.047 mmol) and Pd₂(dba)₃ (0.086 g, 0.094 mmol) and heating at 100° C. for 6 h to afford after workup and purification method-BW, methyl 5-(1-aminoisoquinolin-7-yl)-3-(2-(2-ethoxy-2-oxoethyl)-5-methoxyphenoxy)-2,3-dihydrospiro[indene-1,4′-piperidine]-1′-carboxylate (452b) (60 mg, 21% yield) as a pale yellow solid; MS (ES+): 596.3 (M+1).

### Step-3: Preparation of 2-(2-((5-(1-aminoisoquinolin-7-yl)-1′-(methoxycarbonyl)-2,3-dihydrospiro[indene-1,4′-piperidin]-3-yl)oxy)-4-methoxyphenyl)acetic acid (452c)

Compound 452c was prepared according to the procedure reported in step-2 of scheme 1, from methyl 5-(1-aminoisoquinolin-7-yl)-3-(2-(2-ethoxy-2-oxoethyl)-5-methoxyphenoxy)-2,3-dihydrospiro[indene-1,4′-piperidine]-1′-carboxylate (452b) (60 mg, 0.101 mmol) in MeOH (0.8 mL) and THF (0.8 mL) using lithium hydroxide (1N aqueous solution; 403 μl, 0.403 mmol) to afford after workup and purification method-G twice, 2-(2-((5-(1-aminoisoquinolin-7-yl)-1′-(methoxycarbonyl)-2,3-dihydrospiro[indene-1,4′-piperidin]-3-yl)oxy)-4-methoxyphenyl)acetic acid (452c) (5 mg, 9% yield) HCl salt as a white solid; ¹H NMR (300 MHz, DMSO-d₆) δ 13.12 (s, 1H, D₂O exchangeable), 11.99 (s, 1H, D₂O exchangeable), 9.00 (s, 2H, D₂O exchangeable), 8.87 (s, 1H), 8.30 (d, J=8.3 Hz, 1H), 8.03 (d, J=8.5 Hz, 1H), 7.93-7.81 (m, 2H), 7.69 (d, J=6.9 Hz, 1H), 7.57 (d, J=8.0 Hz, 1H), 7.26 (d, J=6.9 Hz, 1H), 7.11 (d, J=8.3 Hz, 1H), 6.85 (d, J=2.4 Hz, 1H), 6.51 (dd, J=8.3, 2.3 Hz, 1H), 6.05-5.81 (m, 1H), 4.14-3.86 (m, 2H), 3.79 (s, 3H), 3.62 (s, 3H), 3.38 (s, 2H), 3.14-2.64 (m, 3H), 2.13-1.99 (m, 1H), 1.95-1.74 (m, 2H), 1.66-1.48 (m, 2H); MS (ES+): 568.3 (M+1); (ES−): 566.2 (M−1).

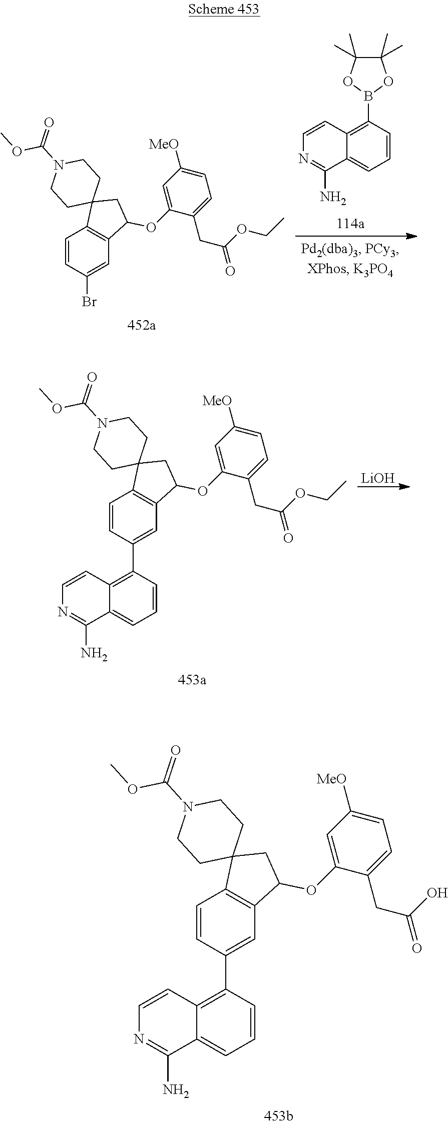

### Preparation of 2-(2-((5-(1-aminoisoquinolin-5-yl)-1′-(methoxycarbonyl)-2,3-dihydrospiro[indene-1,4′-piperidin]-3-yl)oxy)-4-methoxyphenyl)acetic acid (453b)

### Step-1: Preparation of methyl 5-(1-aminoisoquinolin-5-yl)-3-(2-(2-ethoxy-2-oxoethyl)-5-methoxyphenoxy)-2,3-dihydrospiro[indene-1,4′-piperidine]-1′-carboxylate (453a)

Compound 453a was prepared according to the procedure reported in step-3 of scheme 112, from methyl 5-bromo-3-(2-(2-ethoxy-2-oxoethyl)-5-methoxyphenoxy)-2,3-dihydrospiro[indene-1,4′-piperidine]-1′-carboxylate (452a) (0.25 g, 0.470 mmol) in dioxane/2-MeTHF (4 mL) using 5-(4,4,5,5-tetramethyl-1,3,2-dioxaborolan-2-yl)isoquinolin-1-amine (114a) (0.254 g, 0.939 mmol), K₃PO₄ (3M aqueous solution, 313 mL, 0.961 mmol), PCy₃ (0.040 g, 0.141 mmol), XPhos (0.022 g, 0.047 mmol) and Pd₂(dba)₃ (0.086 g, 0.094 mmol) and heating at 100° C. for 6 h to afford after workup and purification using method-BW, methyl 5-(1-aminoisoquinolin-5-yl)-3-(2-(2-ethoxy-2-oxoethyl)-5-methoxyphenoxy)-2,3-dihydrospiro[indene-1,4′-piperidine]-1′-carboxylate (453a) (130 mg, 0.218 mmol, 46.5% yield) as a pale yellow solid; MS (ES+): 596.3 (M+1).

### Step-2: Preparation of 2-(2-((5-(1-aminoisoquinolin-5-yl)-1′-(methoxycarbonyl)-2,3-dihydrospiro[indene-1,4′-piperidin]-3-yl)oxy)-4-methoxyphenyl)acetic acid (453b)

Compound 453b was prepared according to the procedure reported in step-2 of scheme 1, from methyl 5-(1-aminoisoquinolin-5-yl)-3-(2-(2-ethoxy-2-oxoethyl)-5-methoxyphenoxy)-2,3-dihydrospiro[indene-1,4′-piperidine]-1′-carboxylate (453a) (130 mg, 0.218 mmol) in MeOH (1.6 mL) and THF (1.6 mL) using lithium hydroxide (1N aqueous solution; 873 μl, 0.873 mmol) to afford after workup and purification method-G, 2-(2-((5-(1-aminoisoquinolin-5-yl)-1′-(methoxycarbonyl)-2,3-dihydrospiro[indene-1,4′-piperidin]-3-yloxy)-4-methoxyphenyl)acetic acid (453b)(57 mg, 46% yield) HCl salt as a white solid; ¹H NMR (300 MHz, DMSO-d₆) δ 13.32 (s, 1H, D₂O exchangeable), 11.96 (s, 1H, D₂O exchangeable), 9.17 (s, 2H, D₂O exchangeable), 8.61 (d, J=8.2 Hz, 1H), 7.95-7.78 (m, 2H), 7.61 (d, J=7.3 Hz, 1H), 7.55 (d, J=7.9 Hz, 1H), 7.47-7.32 (m, 2H), 7.08 (d, J=8.3 Hz, 1H), 6.96 (d, J=7.2 Hz, 1H), 6.82 (d, J=2.4 Hz, 1H), 6.48 (dd, J=8.3, 2.3 Hz, 1H), 6.02-5.88 (m, 1H), 4.15-3.91 (m, 2H), 3.76 (s, 3H), 3.63 (s, 3H), 3.29 (s, 2H), 3.23-2.85 (m, 2H), 2.76 (dd, J=13.7, 6.9 Hz, 1H), 2.08 (dd. J=13.7, 4.4 Hz, 1H), 1.98-1.72 (m, 2H), 1.72-1.50 (m, 2H); MS (ES+): 568.2 (M+1); (ES−): 566.2 (M−1).

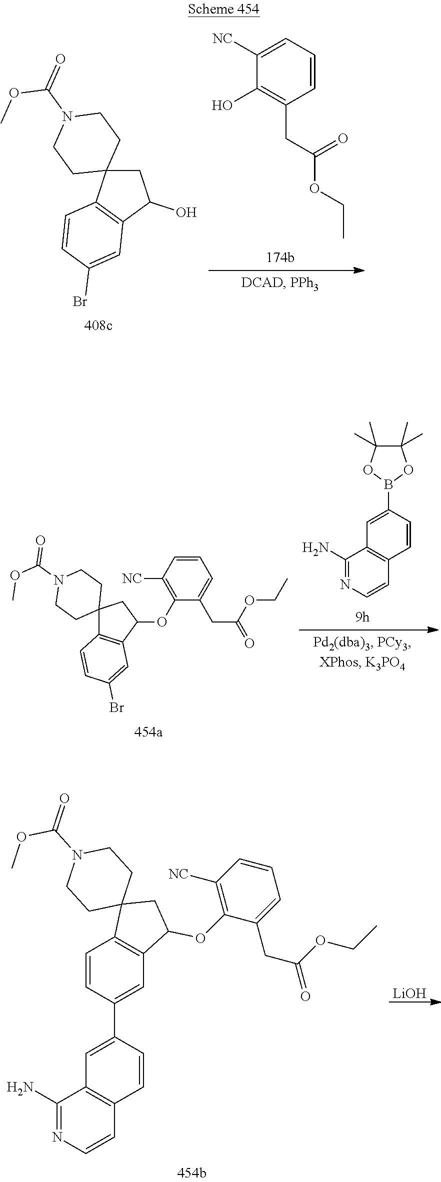

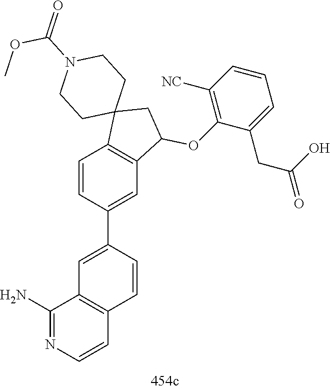 [SMILES: COC(=O)N1CCC2(CC1)CC(Oc1c(C#N)cccc1CC(=O)O)c1cc(-c3ccc4ccnc(N)c4c3)ccc12]

### Preparation of 2-(2-((5-(1-aminoisoquinolin-7-yl)-1′-(methoxycarbonyl)-2,3-dihydrospiro[indene-1,4′-piperidin]-3-yl)oxy)-3-cyanophenyl)acetic acid (454c)

### Step-1: Preparation of methyl 5-bromo-3-(2-cyano-6-(2-ethoxy-2-oxoethyl)phenoxy)-2,3-dihydrospiro[indene-1,4′-piperidine]-1′-carboxylate (454a)

Compound 454a was prepared according to the procedure reported in step-2 of scheme 2, from methyl 5-bromo-3-hydroxy-2,3-dihydrospiro[indene-1,4′-piperidine]-1′-carboxylate (408c) (0.6 g, 1.764 mmol) in DCM (30 mL) using PPh₃ (0.555 g, 2.116 mmol), ethyl 2-(3-cyano-2-hydroxyphenyl)acetate (174b) (0.434 g, 2.116 mmol) and a solution of DCAD (0.777 g, 2.116 mmol) in DCM (10 mL) to afford after workup and purification using method-O, methyl 5-bromo-3-(2-cyano-6-(2-ethoxy-2-oxoethyl)phenoxy)-2,3-dihydrospiro[indene-1,4′-piperidine]-1′-carboxylate (454a) (0.89 g, 96% yield) as a clear gel; MS (ES+): 549.1 (M+Na).

### Step-2: Preparation of methyl 5-(1-aminoisoquinolin-7-yl)-3-(2-cyano-6-(2-ethoxy-2-oxoethyl)phenoxy)-2,3-dihydrospiro[indene-1,4′-piperidine]-1′-carboxylate (454b)

Compound 454b was prepared according to the procedure reported in step-3 of scheme 112, from methyl 5-bromo-3-(2-cyano-6-(2-ethoxy-2-oxoethyl)phenoxy)-2,3-dihydrospiro[indene-1,4′-piperidine]-1′-carboxylate (454a) (0.41 g, 0.777 mmol) in dioxane/2-MeTHF (5 mL) using 7-(4,4,5,5-tetramethyl-1,3,2-dioxaborolan-2-yl)isoquinolin-1-amine (9h) (0.420 g, 1.555 mmol), K₃PO₄ (3M aqueous solution, 0.518 mL, 1.555 mmol), PCy₃ (0.065 g, 0.233 mmol), XPhos (0.037 g, 0.078 mmol) and Pd₂(dba)₃ (0.142 g, 0.155 mmol) and heating at 100° C. for 4 h to afford after workup and purification method-BW, methyl 5-(1-aminoisoquinolin-7-yl)-3-(2-cyano-6-(2-ethoxy-2-oxoethyl)phenoxy)-2,3-dihydrospiro[indene-1,4′-piperidine]-1′-carboxylate (454b)(172 mg, 38% yield) as a pale yellow solid; MS (ES+): 591.3 (M+1).

### Step-3: Preparation of 2-(2-((5-(1-aminoisoquinolin-7-yl)-1′-(methoxycarbonyl)-2,3-dihydrospiro[indene-1,4′-piperidin]-3-yl)oxy)-3-cyanophenyl)acetic acid (454c)

Compound 454c was prepared according to the procedure reported in step-2 of scheme 1, from methyl 5-(1-aminoisoquinolin-7-yl)-3-(2-cyano-6-(2-ethoxy-2-oxoethyl)phenoxy)-2,3-dihydrospiro[indene-1,4′-piperidine]-1′-carboxylate (454b) (170 mg, 0.288 mmol) in MeOH (2.5 mL) and THF (2.5 mL) using lithium hydroxide (1N aqueous solution; 1151 μl, 1.151 mmol) to afford after workup and purification method-G, 2-(2-((5-(1-aminoisoquinolin-7-yl)-1′-(methoxycarbonyl)-2,3-dihydrospiro[indene-1,4′-piperidin]-3-yl)oxy)-3-cyanophenyl)acetic acid (454c) (83 mg, 51% yield) HCl salt as a white solid; ¹H NMR (300 MHz, DMSO-d₆) δ 13.24 (s, 1H, D₂O exchangeable), 12.48 (s, 1H, D₂O exchangeable), 9.11 (s, 2H, D₂O) exchangeable), 8.85 (s, 1H), 8.10 (dd, J=8.5, 1.6 Hz, 1H), 8.05-7.89 (m, 2H), 7.79 (dd, J=7.7, 1.7 Hz, 1H), 7.69 (d, J=6.9 Hz, 1H), 7.66-7.61 (m, 2H), 7.56 (d, J=1.7 Hz, 1H), 7.36-7.21 (m, 2H), 5.97-5.84 (m, 1H), 4.17-3.92 (m, 2H), 3.64 (s, 3H), 3.56-3.38 (m, 2H), 3.22-2.88 (m, 2H), 2.59-2.53 (m, 1H), 2.47-2.39 (m, 1H), 2.00-1.75 (m, 3H), 1.45 (d, J=12.8 Hz, 1H); MS (ES+): 563.2 (M+1); (ES−): 561.2 (M−1); FT-1R: 2230.8 cm⁻¹.

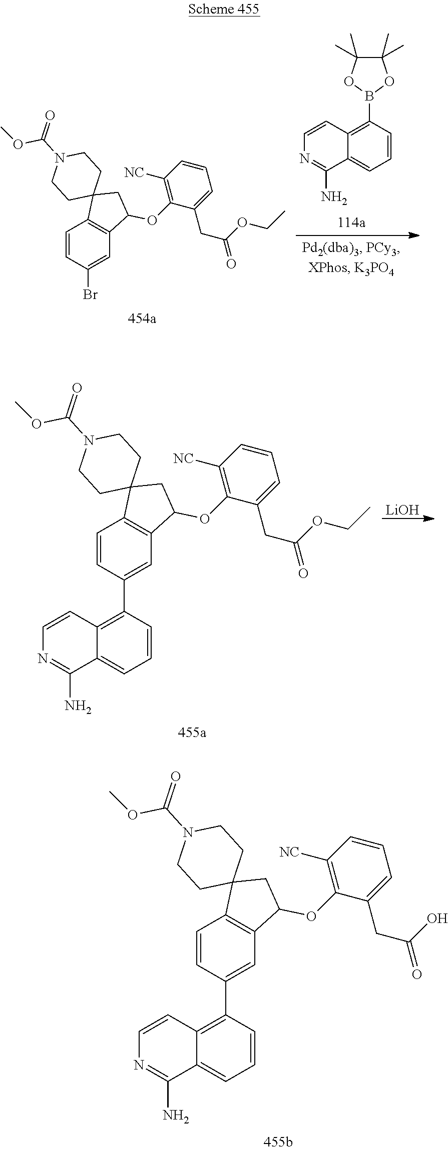

### Preparation of 2-(2-((5-(1-aminoisoquinolin-5-yl)-1′-(methoxycarbonyl)-2,3-dihydrospiro[indene-1,4′-piperidin]-3-yl)oxy)-3-cyanophenyl)acetic acid (455b)

### Step-1: Preparation of methyl 5-(1-aminoisoquinolin-5-yl)-3-(2-cyano-6-(2-ethoxy-2-oxoethyl)phenoxy)-2,3-dihydrospiro[indene-1,4′-piperidine]-1′-carboxylate (455a)

Compound 455a was prepared according to the procedure reported in step-3 of scheme 112, from methyl 5-bromo-3-(2-cyano-6-(2-ethoxy-2-oxoethyl)phenoxy)-2,3-dihydrospiro[indene-1,4′-piperidine]-1′-carboxylate (454a) (0.41 g, 0.777 mmol) in dioxane/2-MeTHF (5 mL) using 5-(4,4,5,5-tetramethyl-1,3,2-dioxaborolan-2-yl)isoquinolin-1-amine (114a) (0.420 g, 1.555 mmol), K₃PO₄ (3M aqueous solution, 0.518 mL, 1.555 mmol), PCy₃ (0.065 g, 0.233 mmol), XPhos (0.037 g, 0.078 mmol). Pd₂(dba)₃ (0.142 g, 0.155 mmol) and heating at 100° C. for 4 h to afford after workup and purification using method-BW, methyl 5-(1-aminoisoquinolin-5-yl)-3-(2-cyano-6-(2-ethoxy-2-oxoethyl)phenoxy)-2,3-dihydrospiro[indene-1,4′-piperidine]-1′-carboxylate (455a) (130 mg, 46.5% yield) as a pale yellow solid; MS (ES+): 596.3 (M+1).

### Step-2: Preparation of 2-(2-((5-(1-aminoisoquinolin-5-yl)-1′-(methoxycarbonyl)-2,3-dihydrospiro[indene-1,4′-piperidin]-3-yl)oxy)-3-cyanophenyl)acetic acid (455b)

Compound 455b was prepared according to the procedure reported in step-2 of scheme 1, from methyl 5-(1-aminoisoquinolin-5-yl)-3-(2-cyano-6-(2-ethoxy-2-oxoethyl)phenoxy)-2,3-dihydrospiro[indene-1,4′-piperidine]-1′-carboxylate (455a) (220 mg, 0.372 mmol) in MeOH (3 mL) and THF (3 mL) using lithium hydroxide (1N aqueous solution; 1490 μl, 1.490 mmol) to afford after workup and purification using method-G, 2-(2-((5-(1-aminoisoquinolin-5-yl)-1′-(methoxycarbonyl)-2,3-dihydrospiro[indene-1,4′-piperidin]-3-yl)oxy)-3-cyanophenyl)acetic acid (455b) (147 mg, 70% yield) HCl salt as a white solid; H NMR (300 MHz, DMSO-d₆) δ 13.38 (s, 1H, D₂O exchangeable), 12.47 (s, 1H, D₂O exchangeable), 9.15 (s, 2H, D₂O exchangeable), 8.65-8.51 (m, 1H), 7.85-7.71 (m, 3H), 7.69-7.57 (m, 3H), 7.46 (dd, J=7.9, 1.7 Hz, 1H), 7.29-7.20 (m, 1H), 6.99 (d, J=1.6 Hz, 1H), 6.80 (d, J=7.2 Hz, 1H), 5.85 (dd, J=6.7, 2.3 Hz, 1H), 4.22-3.94 (m, 2H), 3.64 (s, 3H), 3.60-3.37 (m, 2H), 3.25-2.90 (m, 2H), 2.69-2.56 (m, 1H), 2.48-2.38 (m, 1H), 2.05-1.80 (m, 3H), 1.55-1.43 (m, 1H); MS (ES+): 563.3 (M+1); (ES−): 561.2 (M−1); FT-1R: 2230.8 cm⁻¹.

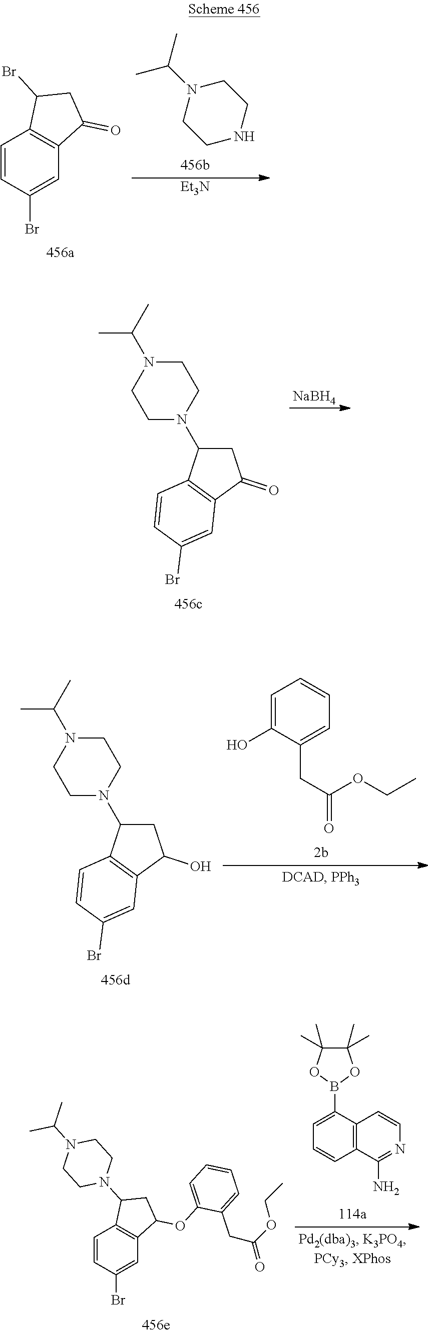

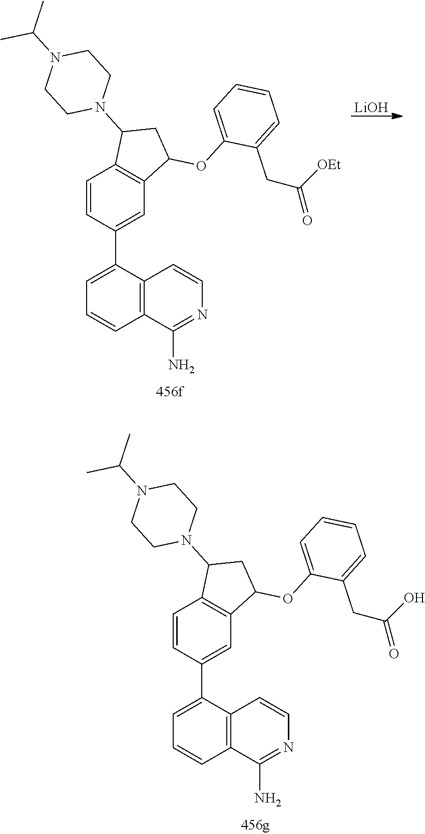 [SMILES: CCOC(=O)Cc1ccccc1OC1CC(N2CCN(C(C)C)CC2)c2ccc(-c3cccc4c(N)nccc34)cc21 ; "LiOH" ; CC(C)N1CCN(C2CC(Oc3ccccc3CC(=O)O)c3cc(-c4cccc5c(N)nccc45)ccc32)CC1]

### Preparation of 2-(2-((6-(1-aminoisoquinolin-5-yl)-3-(4-isopropylpiperazin-1-yl)-2,3-dihydro-1H-inden-1-yl)oxy)phenyl)acetic acid (456g)

### Step-1: Preparation of 6-bromo-3-(4-isopropylpiperazin-1-yl)-2,3-dihydro-1H-inden-1-one (456c)

To a stirred solution of 3,6-dibromo-2,3-dihydro-1H-inden-1-one (456a) (3.0 g, 10.42 mmol; CAS #1443227-18-6) in DCM (10 mL) under nitrogen was added Et₃N (4.26 mL, 31.26 mmol), 1-isopropylpiperazine (456b) (2.0 g, 15.63 mmol; CAS #4318-42-7) and stirred at RT for 4 h. The reaction mixture was diluted with water and DCM and extracted 2× with DCM. The combined organics was dried, filtered, and concentrated in vacuum. The residue obtained was purified using method-BY, to afford 6-bromo-3-(4-isopropylpiperazin-1-yl)-2,3-dihydro-1H-inden-1-one (456c) (1.2 g, 35% yield) as colorless syrup. ¹H NMR (400 MHz, CDCl₃): δ 7.87 (d, J=1.6 Hz, 1H), 7.74 (dd, J=2.0, 1.6 Hz, 1H), 7.59 (d, J=8.4 Hz, 1H), 4.51 (dd, J=3.2, 3.6 Hz, 1H), 2.85-2.37 (m, 9H), 2.38 (t, J=7.6 Hz, 2H), 1.04 (d, J=6.8, Hz, 6H); MS (ES+): 337.0 (M+1).

### Step-2: Preparation of 6-bromo-3-(4-isopropylpiperazin-1-yl)-2,3-dihydro-1H-inden-1-ol (456d)

Compound 456d was prepared according to the procedure reported in step-1 of scheme 205, from 6-bromo-3-(4-isopropylpiperazin-1-yl)-2,3-dihydro-1H-inden-1-one (456c) (1.2 g, 3.56 mmol) in anhydrous MeOH (24 mL) using sodium borohydride (0.269 g, 7.12 mmol) to afford after work up and purification using purification method-BY, 6-bromo-3-(4-isopropylpiperazin-1-yl)-2,3-dihydro-1H-inden-1-ol (456d) (1.0 g, 83% yield) as a colorless syrup; ¹H NMR (400 MHZ, DMSO-d₆): δ 7.46 (s, 1H), 7.41 (d, J=8.4, Hz, 1H), 7.18 (d, J=8.0 Hz, 1H), 5.49 (d, J=5.6 Hz, 1H), 4.87 (q, J=6.8 Hz, 2H), 4.09 (t, J=8.0 Hz, 1H), 2.67-2.32 (m, 9H), 1.77-1.70 (m, 1H), 1.02 (d, J=5.6 Hz, 6H); MS (ES+): 339.20 (M+1).

### Step-3: Preparation of ethyl 2-(2-((6-bromo-3-(4-isopropylpiperazin-1-yl)-2,3-dihydro-1H-inden-1-yl)oxy)phenyl)acetate (456e)

Compound 456e was prepared according to the procedure reported in step-2 of scheme 2, from 6-bromo-3-(4-isopropylpiperazin-1-yl)-2,3-dihydro-1H-inden-1-ol (456d) (1.0 g, 2.94 mmol) in DCM (20 mL) using PPh₃ (1.54 g, 5.88 mmol), ethyl 2-(2-hydroxyphenyl)acetate (2b) (0.637 g, 3.52 mmol) and a solution of DCAD (2.16 g, 5.88 mmol) in DCM (10 mL) to afford after workup and purification method-BY, ethyl 2-(2-((6-bromo-3-(4-isopropylpiperazin-1-yl)-2,3-dihydro-1H-inden-1-yl)oxy)phenyl)acetate (456e) (0.8 g, 54% yield) as a colorless gel; ¹H NMR (400 MHz, DMSO-d₆): δ 7.56 (d, J=7.2 Hz, 1H), 7.47 (s, 1H), 7.29 (d, J=8.0 Hz, 1H), 7.19 (d, J=9.2 Hz, 1H), 7.09-7.04 (m, 1H), 6.92 (t, J=7.6 Hz, 1H), 6.79-6.70 (m, 1H), 5.83 (t, J=6.4 Hz, 1H), 4.46-4.42 (m, 1H), 3.99-2.88 (m, 2H), 3.46 (s, 2H), 2.66-2.25 (m, 10H), 1.97-1.90 (m, 1H), 1.05 (d, J=6.8 Hz, 3H), 1.02 (d, J=6.4 Hz, 6H); MS (ES+): 503.30 (M+3).

### Step-4: Preparation of ethyl 2-(2-((6-(1-aminoisoquinolin-5-yl)-3-(4-isopropylpiperazin-1-yl)-2,3-dihydro-1H-inden-1-yl)oxy)phenyl)acetate (456f)

Compound 456f was prepared according to the procedure reported in step-3 of scheme 112, from ethyl 2-(2-((6-bromo-3-(4-isopropylpiperazin-1-yl)-2,3-dihydro-1H-inden-1-yl)oxy)phenyl)acetate (456e) (400 mg, 0.798 mmol) in dioxane/2-MeTHF (10 mL) using 5-(4,4,5,5-tetramethyl-1,3,2-dioxaborolan-2-yl)isoquinolin-1-amine (114a) (323 mg, 1.19 mmol). K₃PO₄ (338 mg, 1.596 mmol), PCy₃ (111 mg, 0.399 mmol), XPhos (38 mg, 0.039 mmol) and Pd₂(dba)₃ (146 mg, 0.715 mmol) and heating at 100° C. for 16 h to afford after workup and purification Method-BZ, ethyl 2-(2-((6-(1-aminoisoquinolin-5-yl)-3-(4-isopropylpiperazin-1-yl)-2,3-dihydro-1H-inden-1-yl)oxy)phenyl)acetate (456f) (250 mg, 56% yield) as a brown solid; MS (ES+): 565.01 (M+1).

### Step-5: Preparation of 2-(2-((6-(1-aminoisoquinolin-5-yl)-3-(4-isopropylpiperazin-1-yl)-2,3-dihydro-1H-inden-1-yl)oxy)phenyl)acetic acid (456g)

Compound 456g was prepared according to the procedure reported in step-2 of scheme 1, from ethyl 2-(2-((6-(1-aminoisoquinolin-5-yl)-3-(4-isopropylpiperazin-1-yl)-2,3-dihydro-1H-inden-1-yl)oxy)phenyl)acetate (456f) (250 mg, 0.448 mmol) in MeOH (4 mL), THF (4 mL) and water (2 mL) using lithium hydroxide (55 mg, 0.134 mmol) and stirring overnight to afford after work up and purification method-CA, 2-(2-((6-(1-aminoisoquinolin-5-yl)-3-(4-isopropylpiperazin-1-yl)-2,3-dihydro-1H-inden-1-yl)oxy)phenyl)acetic acid (456g) (92 mg, 39% yield) TFA salt as an off-white solid; ¹H NMR (300 MHz, DMSO-d₆) δ 13.25 (s, 1H, D₂O exchangeable), 11.96 (s, 1H, D₂O exchangeable), 9.20 (s, 3H, D₂O exchangeable), 8.59 (dd, J=7.8, 1.6 Hz, 1H), 7.96-7.79 (m, 2H), 7.65-7.55 (m, 2H), 7.55-7.42 (m, 2H), 7.33-7.17 (m, 3H), 6.97-6.86 (m, 2H), 5.97 (t, J=5.4 Hz, 1H), 4.82-4.65 (m, 1H), 3.52-2.55 (m, 12H), 2.32-2.11 (m, 1H), 1.26 (d, J=6.5 Hz, 6H); MS (ES+): 537.20 (M+1); Analysis calculated for C₃₃H₃₆N₄O₃·2H₂O·3.25CF₃COOH: C, 50.30; H, 4.62; F, 19.64; N, 5.94; Found: C, 49.99; H, 4.25; N, 5.93.

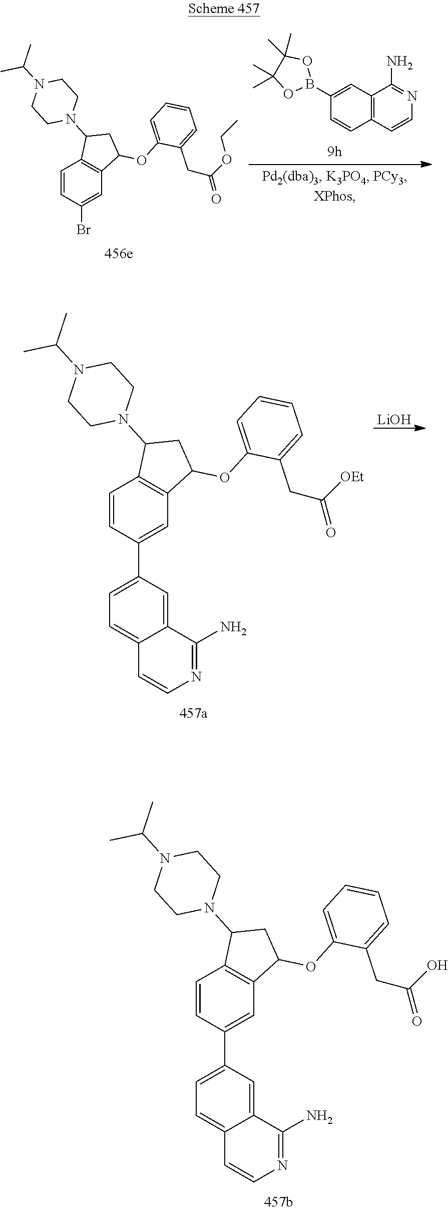

### Preparation of 2-(2-(6-(1-aminoisoquinolin-7-yl)-3-(4-isopropylpiperazin-1-yl)-2,3-dihydro-1H-inden-1-yl)oxy)phenyl)acetic acid (457b)

### Step-1: Preparation of ethyl 2-(2-((6-(1-aminoisoquinolin-7-yl)-3-(4-isopropylpiperazin-1-yl)-2,3-dihydro-1H-inden-1-yl)oxy)phenyl)acetate (457a)

Compound 457a was prepared according to the procedure reported in step-3 of scheme 112, from ethyl 2-(2-((6-bromo-3-(4-isopropylpiperazin-1-yl)-2,3-dihydro-1H-inden-1-yl)oxy)phenyl)acetate (456e) (400 mg, 0.798 mmol) in dioxane/2-MeTHF (10 mL) using 7-(4,4,5,5-tetramethyl-1,3,2-dioxaborolan-2-yl)isoquinolin-1-amine (9h) (323 mg, 1.19 mmol), K₃PO₄ (338 mg, 1.596 mmol), PCy₃ (111 mg, 0.399 mmol), XPhos (38 mg, 0.039 mmol), Pd₂(dba)₃ (146 mg, 0.715 mmol) and heating at 100° C. for 16 h to afford after workup and purification using method-BZ, ethyl 2-(2-((6-(1-aminoisoquinolin-7-yl)-3-(4-isopropylpiperazin-1-yl)-2,3-dihydro-1H-inden-1-yl)oxy)phenyl)acetate (457a) (250 mg, 56% yield) as a brown solid; MS (ES+): 565.01 (M+1).

### Step-2: Preparation of 2-(2-(6-(1-aminoisoquinolin-7-yl)-3-(4-isopropylpiperazin-1-yl)-2,3-dihydro-1H-inden-1-yl)oxy)phenyl)acetic acid (457b)

Compound 457b was prepared according to the procedure reported in step-2 of scheme 1, from ethyl 2-(2-((6-(1-aminoisoquinolin-7-yl)-3-(4-isopropylpiperazin-1-yl)-2,3-dihydro-1H-inden-1-yl)oxy)phenyl)acetate (457a) (250 mg, 0.448 mmol) in MeOH (4 mL), THF (4 mL) and water (2 mL) using lithium hydroxide (55 mg, 0.134 mmol) and stirring overnight to afford after work up and purification using method-CB, 2-(2-((6-(1-aminoisoquinolin-7-yl)-3-(4-isopropylpiperazin-1-yl)-2,3-dihydro-1H-inden-1-yl)oxy)phenyl)acetic acid (457b) (21 mg, 11% yield) TFA salt as an off-white solid; ¹H NMR (300 MHz, DMSO-d₆) δ 13.08 (s, 1H, D₂O exchangeable), 12.12 (s, 1H, D₂O exchangeable), 9.07 (s, 3H, D₂O exchangeable), 8.89 (d, J=1.6 Hz, 1H), 8.35 (d, J=8.5 Hz, 1H), 8.07 (d, J=8.5 Hz, 1H), 8.00-7.88 (m, 2H), 7.70 (d, J=7.0 Hz, 1H), 7.58 (d, J=8.2 Hz, 1H), 7.36-7.18 (m, 4H), 6.95 (t, J=7.1 Hz, 1H), 6.03-5.92 (m, 1H), 4.73-4.64 (m, 1H), 3.57-2.55 (m, 12H), 2.21-2.09 (m, 1H), 1.26 (d, J=6.8 Hz, 6H); MS (ES+): 537.20 (M+1).

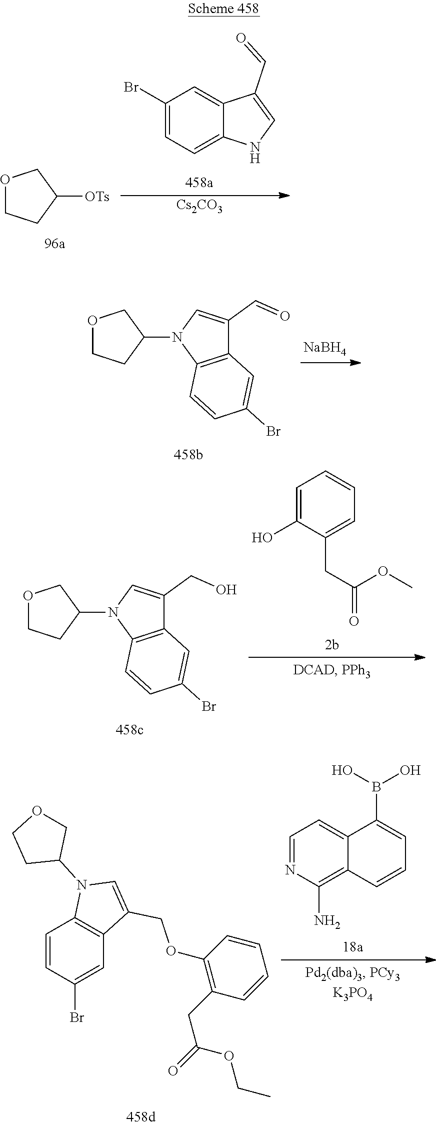

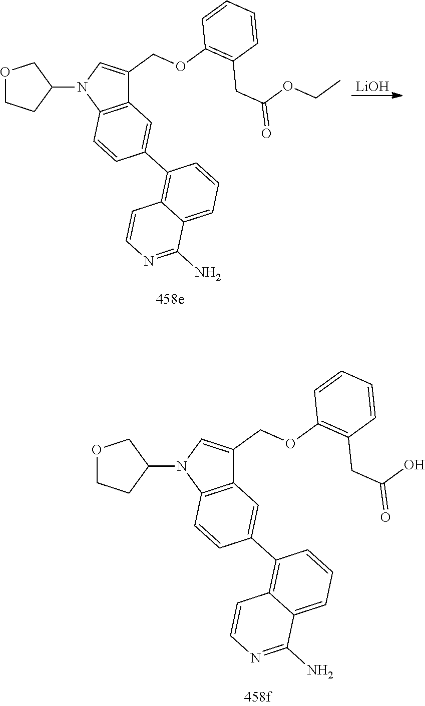

### Preparation of 2-(2-((5-(1-aminoisoquinolin-5-yl)-1-(tetrahydrofuran-3-yl)-1H-indol-3-yl)methoxy)phenyl)acetic acid (458f)

### Step-1: Preparation of 5-bromo-1-(tetrahydrofuran-3-yl)-1H-indole-3-carbaldehyde (458b)

Compound 458b was prepared according to the procedure reported in step-2 of scheme 86, from 5-bromo-1H-indole-3-carbaldehyde (458a) (6.4 g, 28.56 mmol; CAS #877-03-2) in DMF (77 mL) using Cs₂CO₃ (18.61, 57.126 mmol) and tetrahydrofuran-3-yl 4-methylbenzenesulfonate (96a) (7.69 g, 31.74 mmol) to afford after workup and purification using column chromatography [silica gel, eluting with EtOAc in n-heptane from 0-60%] 5-bromo-1-(tetrahydrofuran-3-yl)-1H-indole-3-carbaldehyde (458b) (6 g, 64% yield) as a light pink solid; ¹H NMR (300 MHz, DMSO-d₆) δ 9.92 (d, J=0.8 Hz, 1H), 8.41 (s, 1H), 8.25 (d, J=2.0 Hz, 1H), 7.72 (d, J=8.8 Hz, 1H), 7.48 (dd. J=8.8, 2.0 Hz, 1H), 5.41-5.28 (m, 1H), 4.18-4.05 (m, 1H), 4.05-3.93 (m, 2H), 3.89-3.77 (m, 1H), 2.64-2.53 (m, 1H), 2.26-2.13 (m, 1H).

### Step-2: Preparation of (5-bromo-1-(tetrahydrofuran-3-yl)-1H-indol-3-yl)methanol (458c)

Compound 458c was prepared according to the procedure reported in step-1 of scheme 205, from 5-bromo-1-(tetrahydrofuran-3-yl)-1H-indole-3-carbaldehyde (458b) (2 g, 6.80 mmol) in anhydrous MeOH (5 mL) using sodium borohydride (0.39 g, 10.2 mmol) to afford after work up (5-bromo-1-(tetrahydrofuran-3-yl)-1H-indol-3-yl)methanol (458c) (2 g) as a colorless liquid and was used as such for the next step; ¹H NMR (300 MHz, DMSO-d₆) δ 7.80 (d, J=2.0 Hz, 1H), 7.51 (d, J=8.7 Hz, 1H), 7.37 (s, 1H), 7.26 (dd, J=8.7, 2.0 Hz, 1H), 5.27-5.15 (m, 1H), 4.89 (t, J=5.5 Hz, 1H), 4.65-4.54 (m, 2H), 4.14-4.01 (m, 1H), 3.99-3.76 (m, 3H), 2.49-2.37 (m, 1H), 2.15-1.97 (m, 1H).

### Step-3: Preparation of ethyl 2-(2-((5-bromo-1-(tetrahydrofuran-3-yl)-1H-indol-3-yl)methoxy)phenyl)acetate (458d)

Compound 458d was prepared according to the procedure reported in step-2 of scheme 2, from (5-bromo-1-(tetrahydrofuran-3-yl)-1H-indol-3-yl)methanol (458c) (1.9 g, 6.42 mmol) in DCM (80 mL) using PPh₃ (2.03 g, 7.70 mmol), ethyl 2-(2-hydroxyphenyl)acetate (2b) (1.213 g, 6.74 mmol) and a solution of DCAD (2.83 g, 7.70 mmol) in DCM (40 mL) to afford after workup and purification using column chromatography [silica gel, eluting with EtOAc in n-heptane from 0-15%] ethyl 2-(2-((5-bromo-1-(tetrahydrofuran-3-yl)-1H-indol-3-yl)methoxy)phenyl)acetate (458d) (1.5 g, 51% yield) as a white solid; ¹H NMR (300 MHZ, DMSO-d₆) δ 7.65-7.54 (m, 1H), 7.50-7.17 (m, 6H), 7.00-6.85 (m, 1H), 5.27-5.17 (m, 1H), 5.08 (s, 2H), 4.19-3.66 (m, 6H), 3.34 (d, J=1.1 Hz, 2H), 2.52-2.42 (m, 1H), 2.17-2.00 (m, 1H), 1.03-0.80 (m, 3H).

### Step-4: Preparation of ethyl 2-(2-((5-(1-aminoisoquinolin-5-yl)-1-(tetrahydrofuran-3-yl)-1H-indol-3-yl)methoxy)phenyl)acetate (458e)

Compound 458e was prepared according to the procedure reported in step-5 of scheme 1, from ethyl 2-(2-((5-bromo-1-(tetrahydrofuran-3-yl)-1H-indol-3-yl)methoxy)phenyl)acetate (458d) (1.0 g, 2.18 mmol) in 1,4-dioxane (10 mL) and THF (10 mL) using 1-aminoisoquinolin-5-ylboronic acid (18a) (0.615 g, 3.273 mmol), K₃PO₄ (1.852 g, 8.724 mmol), PCy₃ (0.122 g, 0.436 mmol), Pd₂(dba)₃ (0.199 g, 0.218 mmol) to afford after workup and purification using column chromatography [silica gel, eluting with MeOH in DCM from 0-5%] ethyl 2-(2-((5-(1-aminoisoquinolin-5-yl)-1-(tetrahydrofuran-3-yl)-1H-indol-3-yl)methoxy)phenyl)acetate (458e) (0.2 g, 18% yield) as a white solid; MS (ES+): 522.5 (M+1).

### Step-5: Preparation of 2-(2-((5-(1-aminoisoquinolin-5-yl)-1-(tetrahydrofuran-3-yl)-1H-indol-3-yl)methoxy)phenyl)acetic acid (458f)

Compound 458f was prepared according to the procedure reported in step-2 of scheme 1, from ethyl 2-(2-((5-(1-aminoisoquinolin-5-yl)-1-(tetrahydrofuran-3-yl)-1H-indol-3-yl)methoxy)phenyl)acetate (458e) (0.020 g, 38 μmol) in THF (4 mL) and MeOH (2 mL) using lithium hydroxide (4.6 mg, 0.19 mmol) in water (1 mL) to afford after work up and purification using method-CC, 2-(2-((5-(1-aminoisoquinolin-5-yl)-1-(tetrahydrofuran-3-yl)-1H-indol-3-yl)methoxy)phenyl)acetic acid (458f) (0.0087 g, 46% yield) free base as a white solid; ¹H NMR (300 MHz, DMSO-d₆) δ 8.18 (d, J=8.1 Hz, 1H), 7.75 (d, J=6.1 Hz, 1H), 7.71-7.64 (m, 2H), 7.61-7.55 (m, 1H), 7.54-7.47 (m, 1H), 7.29-7.22 (m, 1H), 7.18-7.09 (m, 2H), 6.88 (d, J=6.1 Hz, 1H), 6.85-6.74 (m, 3H), 5.34-5.26 (m, 1H), 5.24 (s, 2H), 4.17-4.07 (m, 1H), 4.05-3.93 (m, 2H), 3.91-3.80 (m, 1H), 3.33-3.10 (m, 2H), 2.60-2.54 (m, 1H), 2.33-2.03 (m, 1H); MS (ES+): 494.20 (M+1); (ES−): 492.10 (M−1).

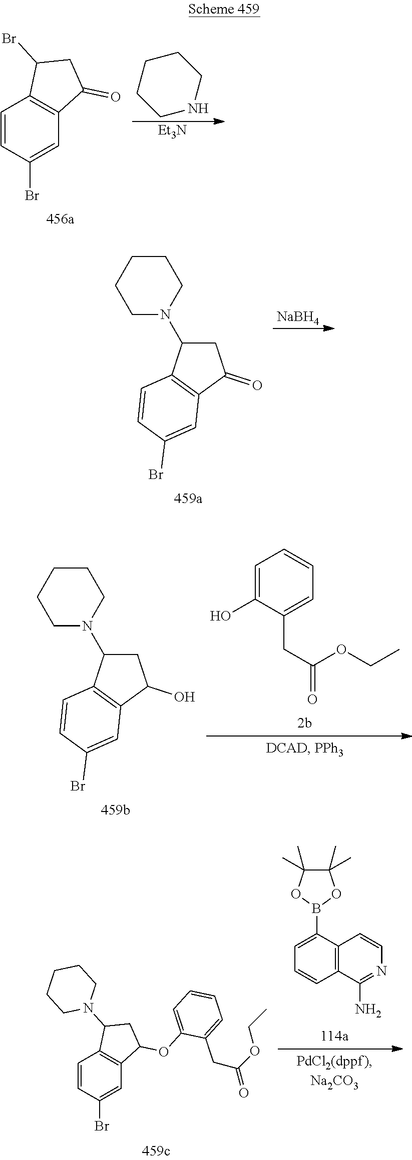

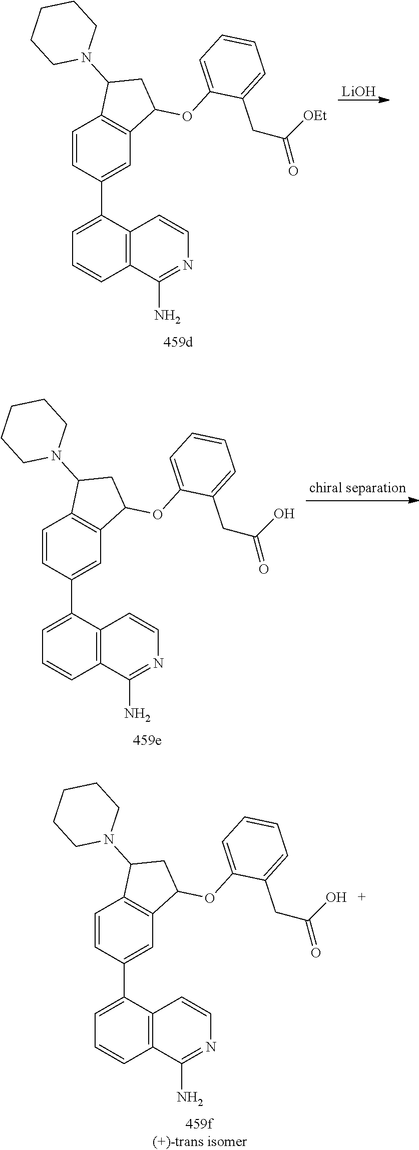

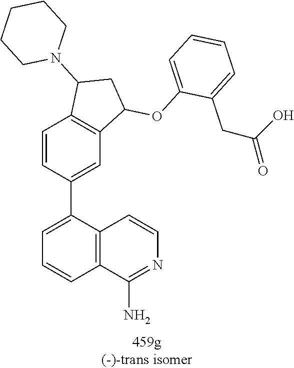 [SMILES: Nc1nccc2c(-c3ccc4c(c3)C(Oc3ccccc3CC(=O)O)CC4N3CCCCC3)cccc12]

### Preparation of (+)-trans 2-(2-(((1R,3R)-6-(1-aminoisoquinolin-5-yl)-3-(piperidin-1-yl)-2,3-dihydro-1H-inden-1-yl)oxy)phenyl)acetic acid (459f) and (−)-trans 2-(2-(((1S,3S)-6-(1-aminoisoquinolin-5-yl)-3-(piperidin-1-yl)-2,3-dihydro-1H-inden-1-yl)oxy)phenyl)acetic acid (459g)

### Step-1: Preparation of 6-bromo-3-(piperidin-1-yl)-2,3-dihydro-1H-inden-1-one (459a)

Compound 459a was prepared according to the procedure reported in step-1 of scheme 456, from 3,6-dibromo-2,3-dihydro-1H-inden-1-one (456a) (1.0 g, 3.46 mmol) in DCM (10 mL) using Et₃N (0.34 mL, 6.92 mmol), piperidine (0.294 g, 3.46 mmol) and stirring at RT for 2 h to afford after work up and purification method-CD, to afford 6-bromo-3-(piperidin-1-yl)-2,3-dihydro-1H-inden-1-one (459a) (800 mg, 78% yield) as a pale-yellow solid; ¹H NMR (400 MHz, CDCl₃): δ 7.87 (s, 1H), 7.76 (d, J=6.8 Hz, 2H), 4.62 (br s, 1H), 2.81-2.35 (m, 6H), 1.62-1.46 (m, 6H); MS (ES+): 295.90 (M+3).

### Step-2: Preparation of 6-bromo-3-(piperidin-1-yl)-2,3-dihydro-1H-inden-1-ol (459b)

Compound 459b was prepared according to the procedure reported in step-1 of scheme 205, from 6-bromo-3-(piperidin-1-yl)-2,3-dihydro-1H-inden-1-one (459a) (800 mg, 2.71 mmol) in anhydrous MeOH (20 mL) using sodium borohydride (0.123 g, 3.25 mmol) to afford after work up 6-bromo-3-(piperidin-1-yl)-2,3-dihydro-1H-inden-1-ol (459b) (800 mg, 87% yield) as a colorless syrup and was used as such for the next step; ¹H NMR (400 MHZ, CDCl₃): δ 7.58 (s, 1H), 7.43 (q, J=4.0 Hz, 1H), 7.25 (d, J=8.04 Hz, 1H), 4.99 (t, J=6.0 Hz, 1H), 3.90 (t, J=6.0 Hz, 1H), 2.53-2.36 (m, 5H), 2.11-2.06 (m, 1H), 1.59-1.55 (m, 5H), 1.46 (d, J=4.8 Hz, 2H); MS (ES+): 296.0 (M+1).

### Step-3: Preparation of ethyl 2-(2-((6-bromo-3-(piperidin-1-yl)-2,3-dihydro-1H-inden-1-yl)oxy)phenyl)acetate (459c)

Compound 459c was prepared according to the procedure reported in step-2 of scheme 2, from 6-bromo-3-(piperidin-1-yl)-2,3-dihydro-1H-inden-1-ol (459b) (1.6 g, 5.42 mmol) in DCM (50 mL) using PPh₃ (2.82 g, 10.88 mmol), ethyl 2-(2-hydroxyphenyl)acetate (2b) (0.975 g, 5.42 mmol) and DCAD (3.96 g, 10.88 mmol) to afford after workup and purification method-CD, ethyl 2-(2-((6-bromo-3-(piperidin-1-yl)-2,3-dihydro-1H-inden-1-yl)oxy)phenyl)acetate (459c) (1.0 g, 41% yield) as a colorless liquid; ¹H NMR (400 MHZ, DMSO-d₆): 7.49-7.45 (m, 2H), 7.32-7.25 (m, 2H), 7.19 (d, J=7.2 Hz, 1H), 6.99-6.92 (m, 2H), 5.73 (br s, 1H), 4.47 (br s, 1H), 4.07-3.99 (m, 2H), 3.55 (q, J=16.0 Hz, 2H), 2.59 (s, 1H), 2.43-2.34 (m, 4H), 2.14 (s, 1H), 1.57-1.47 (m, 6H), 1.15 (t, J=5.6 Hz, 3H); MS (ES+): 459.60 (M+2).

### Step-4: Preparation of ethyl 2-(2-((6-(1-aminoisoquinolin-5-yl)-3-(piperidin-1-yl)-2,3-dihydro-1H-inden-1-yl)oxy)phenyl)acetate (459d)

To a degassed solution of ethyl 2-(2-((6-bromo-3-(piperidin-1-yl)-2,3-dihydro-1H-inden-1-yl)oxy)phenyl)acetate (459c) (800 mg, 1.75 mmol) in water/1,4-Dioxane (10 mL; ratio 1:9) was added Na₂CO₃ (555 mg, 5.25 mmol), 5-(4,4,5,5-tetramethyl-1,3,2-dioxaborolan-2-yl)isoquinolin-1-amine (114a) (708 mg, 2.62 mmol) and PdCl₂(dppf) (73 mg, 0.0875 mmol) and the reaction mixture was degassed with Argon for 15 min, then stirred at 80° C. for 4 h. The suspension was filtered through Celite, washed with DCM, and was concentrated. The residue obtained was purified using method-CE, to give ethyl 2-(2-((6-(1-aminoisoquinolin-5-yl)-3-(piperidin-1-yl)-2,3-dihydro-1H-inden-1-yl)oxy)phenyl)acetate (459d) (405 mg, 45% yield) as a white solid; MS (ES+): 522.07 (M+1).

### Step-5: Preparation of 2-(2-((6-(1-aminoisoquinolin-5-yl)-3-(piperidin-1-yl)-2,3-dihydro-1H-inden-1-yl)oxy)phenyl)acetic acid (459e)

Compound 459e was prepared according to the procedure reported in step-5 of scheme 2, from ethyl 2-(2-((6-(1-aminoisoquinolin-5-yl)-3-(piperidin-1-yl)-2,3-dihydro-1H-inden-1-yl)oxy)phenyl)acetate (459d) (400 mg, 0.76 mmol) in THF/water (10 mL; ratio 9:1) using lithium hydroxide monohydrate (124 mg, 3.04 mmol) and stirring at 45° C. for 18 h. The reaction mixture was concentrated and the residue obtained was acidified (˜pH 5) using HCl (1 N). The obtained solid material was filtered, washed with 80% DCM/heptane and dried to afford racemic 2-(2-((6-(1-aminoisoquinolin-5-yl)-3-(piperidin-1-yl)-2,3-dihydro-1H-inden-1-yl)oxy)phenyl)acetic acid (459e) and was used as such for chiral separation.

### Step-6: Preparation of (+)-trans 2-(2-(((1R,3R)-6-(1-aminoisoquinolin-5-yl)-3-(piperidin-1-yl)-2,3-dihydro-1H-inden-1-yl)oxy)phenyl)acetic acid (459f) and (−)-trans 2-(2-(((1S,3S)-6-(1-aminoisoquinolin-5-yl)-3-(piperidin-1-yl)-2,3-dihydro-1H-inden-1-yl)oxy)phenyl)acetic acid (459g)

Compounds 459f and 459g were obtained by purification of 2-(2-((6-(1-aminoisoquinolin-5-yl)-3-(piperidin-1-yl)-2,3-dihydro-1H-inden-1-yl)oxy)phenyl)acetic acid (459e) using chiral HPLC. [Diacel CHIRAL PAK-IG (250*30 mm, 5μ), Mobile phase A: 0.1% DEA in hexane, Mobile phase B: DCM:MeOH (50:50), Isocratic (A:B): 75:25, Flowrate: 35 mL/min] to give (+)-trans 2-(2-(((1R,3R)-6-(1-aminoisoquinolin-5-yl)-3-(piperidin-1-yl)-2,3-dihydro-1H-inden-1-yl)oxy)phenyl)acetic acid (459f) (53 mg, 14% yield) as a white solid; ¹H NMR (300 MHz, DMSO-d₆) δ 13.46 (s, 1H, D₂O exchangeable), 12.02 (s, 1H, D₂O exchangeable), 11.28 (s, 1H, D₂O exchangeable), 9.18 (s, 2H, D₂O exchangeable), 8.65 (d, J=8.2 Hz, 1H), 8.16 (d, J=8.0 Hz, 1H), 7.94 (dd. J=7.5, 1.2 Hz, 1H), 7.86 (t, J=7.8 Hz, 1H), 7.65 (d, J=7.2 Hz, 1H), 7.62-7.50 (m, 2H), 7.41-7.26 (m, 2H), 7.22 (dd, J=7.4, 1.6 Hz, 1H), 7.03-6.87 (m, 2H), 6.21 (t, J=6.3 Hz, 1H), 5.17 (d, J=8.3 Hz, 1H), 3.51-3.44 (m, 2H), 3.22-3.10 (m, 3H), 3.10-2.92 (m, 2H), 2.41-2.25 (m, 1H), 2.17-1.98 (m, 1H), 1.97-1.66 (m, 4H), 1.61-1.36 (m, 1H); MS (ES+): 494.06 (M+1); Analysis calculated for C₃₁H₃₁N₃O₃, 2H₂O·2.5HCl: C, 59.98; H, 6.09; N, 6.77; Found: C, 59.93; H, 5.88; N, 6.64; [α]_{D}=+2.74 (C=0.25. MeOH); and (−)-trans 2-(2-(((1S,3S)-6-(1-aminoisoquinolin-5-yl)-3-(piperidin-1-yl)-2,3-dihydro-1H-inden-1-yl)oxy)phenyl)acetic acid (459g) (34 mg, 9% yield) as an off-white solid; ¹H NMR (300 MHz, DMSO-d₆) δ 11.37 (s, 1H, D₂O exchangeable), 8.24-8.15 (m, 1H), 7.75 (d, J=6.1 Hz, 1H), 7.58-7.35 (m, 5H), 7.29-7.13 (m, 3H), 6.96-6.71 (m, 4H, 2H D₂O exchangeable), 5.93-5.85 (m, 1H), 4.63-4.52 (m, 1H), 3.38 (s, 2H), 2.77-2.57 (m, 1H), 2.57-2.49 (m, 2H), 2.43-2.29 (m, 2H), 2.14-2.00 (m, 1H), 1.62-1.37 (m, 6H); MS (ES+): 494.06 (M+1); [α]_{D}=−1.46 (C=0.25, MeOH); the relative trans stereochemistry was confirmed by NOESY analysis.

### Example 460

### Kallikrein (KLK) 5 Enzymatic Assay:

Kallikrein-5 (KLK5) is a member of the serine protease family of proteolytic enzymes, confirmed to have trypsin-like activity (Michael I P, et al. Biochemical and enzymatic characterization of human kallikrein 5 (hK5), a novel serine protease potentially involved in cancer progression (J. Biol. Chem. 2005 Apr. 15; 280 (15): 14628-35). Boc-VPR-AMC is a fluorogenic peptide substrate composed of the short peptide chain, Valine-Proline-Arginine (VPR) and the fluorophore, 7-amino-4-methylcoumarin (AMC). KLK5 is able to cleave the fluorogenic peptide substrate Boc-VPR-AMC, releasing the AMC fluorophore, which causes a fluorescent emission in proportion with the enzymatic activity of KLK5. The KLK5 enzymatic activity is monitored by measuring fluorescent emission at wavelengths of 380 nm (excitation) and 460 nm (emission).

The IC₅₀ value of compounds against KLK5 (i.e., the concentration of the compound that inhibits 50% of the enzymatic activity) was calculated according to the procedure reported by R&D systems (Recombinant human KLK5, Catalog Number: 1108-SE), with the exception the pH of the assay was modified from 8.0 to 7.5. Specifically, recombinant human KLK5 (Novoprotein Catalog #C415 or R&D systems Catalog #1108-SE) and fluorogenic peptide Boc-VPR-AMC substrate (R&D Catalog #ES011) were each separately diluted in assay buffer (0.1 M NaH₂PO₄ buffer, pH 7.5). The compounds of the disclosure were dissolved in DMSO at a concentration of 10 mM. A portion of this stock solution was added to the recombinant human KLK5 solution to provide a desired concentration of the compounds and the mixture was incubated at room temperature. The reaction was initiated by the addition of the fluorogenic peptide substrate at room temperature. The final concentration of recombinant human KLK5 was 0.1 μg/well and the final concentration of the fluorogenic substrate was 100 μM (final volume of 100 μL, 1% DMSO). The assays were performed in 96 well F16 Black Maxisorp Plate (Nunc Catalog #475515). Controls included assay buffer alone and enzyme solutions to which DMSO was added. The reaction rates were measured spectrophotometrically by excitation at 380 nm and emission at 460 nm on a Biotek Synergy H1 plate reader.

The reaction rate for enzyme alone was compared to the reaction rate of enzyme in the presence of compound and the percent inhibition was calculated as shown below:

Percent Inhibition=[Rate with inhibitor/Rate without inhibitor)]×100

IC₅₀ values for compounds of the disclosure were determined and are provided in the table below. An IC₅₀ indicates a compound concentration that inhibits 50% of the enzymatic activity. Compounds were tested a minimum of three times. In the table below, three plus symbols (+++) are used to indicate compounds with an IC₅₀ value of less than 1 micromolar; two plus symbols (++) indicate compounds with an IC₅₀ value between 1 and 10 micromolar; and one plus symbol (+) indicates compounds with an IC₅₀ value greater than 10 micromolar.

| Compound | IC₅₀ | Compound | IC₅₀ | Compound | IC₅₀ |
|---|---|---|---|---|---|
| 227i | +++ | 29c | + | 43d | + |
| 228f | +++ | 59d | +++ | 44d | + |
| 7 g | +++ | 28d | +++ | 45c | + |
| 3b | + | 30c | + | 47c | + |
| 1i | ++ | 31c | ++ | 46d | + |
| 2g | + | 32c | + | 48d | +++ |
| 6b | +++ | 33c | + | 8g | +++ |
| 100b | +++ | 34c | + | 9j | +++ |
| 99c | ++ | 35c | + | 10e | +++ |
| 101b | +++ | 36c | + | 109b | ++ |
| 4e | +++ | 85c | + | 49a | + |
| 5e | +++ | 229f | ++ | 50c | + |
| 102c | ++ | 37c | +++ | 51c | + |
| 104d | +++ | 38c | + | 52c | + |
| 103c | +++ | 40b | +++ | 53c | + |
| 105c | + | 39c | + | 54c | + |
| 26c | ++ | 41c | + | 56c | + |
| 27c | + | 42d | +++ | 57b | +++ |
| 58b | +++ | 68c | + | 197j | +++ |
| 111e | +++ | 234c | + | 18c | ++ |
| 11e | +++ | 267g | + | 198b | +++ |
| 110e | +++ | 14f | ++ | 75c | + |
| 12e | + | 15d | + | 113h | ++ |
| 13c | ++ | 16c | ++ | 114d | +++ |
| 13d | + | 196e | +++ | 1156 | ++ |
| 65b | +++ | 69b | +++ | 116b | ++ |
| 60b | +++ | 70c | + | 117c | ++ |
| 63d | ++ | 25c | ++ | 118b | ++ |
| 231f | ++ | 112e | +++ | 268d | ++ |
| 62c | +++ | 71d | +++ | 232b | + |
| 61b | +++ | 72b | +++ | 119h | ++ |
| 66e | +++ | 73d | + | 120c | +++ |
| 233f | ++ | 235b | ++ | 121c | +++ |
| 230d | ++ | 236b | +++ | 122b | ++ |
| 55c | + | 74d | ++ | 123b | ++ |
| 67c | + | 17e | + | 124b | ++ |
| 19c | ++ | 131h | +++ | 153d | + |
| 20b | ++ | 134c | +++ | 154b | ++ |
| 76c | + | 201c | +++ | 149d | ++ |
| 77c | + | 202b | +++ | 150b | +++ |
| 78d | ++ | 203c | +++ | 151d | + |
| 79c | + | 135f | +++ | 152b | ++ |
| 21e | +++ | 136 | ++ | 205e | ++ |
| 22c | +++ | 139f | +++ | 206b | +++ |
| 199d | +++ | 140b | ++ | 23g | ++ |
| 200b | +++ | 137b | ++ | 24b | +++ |
| 125b | +++ | 138b | ++ | 155d | ++ |
| 126c | ++ | 141b | ++ | 156b | ++ |
| 128c | ++ | 269f | +++ | 157d | ++ |
| 127b | ++ | 270b | + | 158b | ++ |
| 129b | ++ | 271c | ++ | 207e | +++ |
| 130c | +++ | 80d | ++ | 208b | +++ |
| 132b | +++ | 81d | +++ | 210b | +++ |
| 133b | + | 204d | ++ | 237c | + |
| 209e | +++ | 214c | + | 220d | ++ |
| 142b | +++ | 163c | +++ | 172d | ++ |
| 211e | ++ | 164c | ++ | 173b | +++ |
| 82e | + | 165b | +++ | 223b | +++ |
| 83e | ++ | 166d | +++ | 225b | +++ |
| 159g | ++ | 167b | +++ | 224b | +++ |
| 160b | ++ | 215d | +++ | 226b | +++ |
| 161c | +++ | 216b | +++ | 272f | ++ |
| 162b | +++ | 217e | + | 273e | +++ |
| 143d | +++ | 218b | ++ | 174c | ++ |
| 144c | +++ | 168c | +++ | 175b | ++ |
| 145b | +++ | 169c | +++ | 256f | ++ |
| 146b | +++ | 170c | +++ | 257b | +++ |
| 147c | +++ | 171b | +++ | 258a | +++ |
| 148b | +++ | 221d | ++ | 252h | +++ |
| 84f | +++ | 222b | ++ | 253b | +++ |
| 212c | ++ | 219d | ++ | 259c | +++ |
| 213c | + | 219c | ++ | 260b | +++ |
| 261b | +++ | 178f | +++ | 88h | +++ |
| 254c | +++ | 182c | +++ | 89b | +++ |
| 238d | +++ | 183b | +++ | 190e | +++ |
| 262e | +++ | 245h | +++ | 191b | +++ |
| 64d | +++ | 246b | +++ | 192e | +++ |
| 239f | +++ | 243h | +++ | 193b | +++ |
| 240b | +++ | 244b | +++ | 90a | +++ |
| 249b | +++ | 241f | +++ | 91a | +++ |
| 250b | +++ | 242b | +++ | 92b | +++ |
| 251c | +++ | 179b | +++ | 93b | +++ |
| 176a | +++ | 184e | +++ | 94e | +++ |
| 177a | ++ | 185b | +++ | 95b | +++ |
| 180g | ++ | 187b | +++ | 106g | +++ |
| 181b | +++ | 188e | +++ | 107d | +++ |
| 255d | +++ | 189b | +++ | 108c | +++ |
| 263d | +++ | 186e | +++ | 96g | +++ |
| 247c | +++ | 86h | +++ | 97a | +++ |
| 248b | +++ | 87c | +++ | 98a | +++ |

| Compound | IC₅₀ | Compound | IC₅₀ |
|---|---|---|---|
| 194g | +++ | 281g | +++ |
| 195b | +++ | 282b | +++ |
| 264d | +++ | 283g | +++ |
| 265h | +++ | 284b | +++ |
| 266b | +++ | 300d | + |
| 274e | ++ | 324d | + |
| 275b | + | 325b | +++ |
| 276c | +++ | 326d | +++ |
| 277b | +++ | 327d | +++ |
| 278c | ++ | 328d | ++ |
| 280a | ++ | 329d | ++ |
| 279g | +++ | | |

| Compound | IC₅₀ | Compound | IC₅₀ | Compound | IC₅₀ |
|---|---|---|---|---|---|
| 285d | ++ | 301f | +++ | 308b | +++ |
| 286a | ++ | 302b | +++ | 309c | +++ |
| 287a | ++ | 297d | +++ | 312c | +++ |
| 288d | +++ | 298c | +++ | 313b | +++ |
| 289b | +++ | 296b | +++ | 310c | +++ |
| 290d | +++ | 305b | +++ | 311b | +++ |
| 291b | +++ | 299b | +++ | 345e | +++ |
| 292d | ++ | 303c | +++ | 343i | +++ |
| 293b | +++ | 304b | +++ | 344b | +++ |
| 294c | +++ | 306c | +++ | 342b | +++ |
| 295c | +++ | 307c | +++ | 340i | +++ |
| 341e | +++ | 352c | +++ | 384b | +++ |
| 346b | +++ | 354f | +++ | 392f | +++ |
| 347d | +++ | 366c | +++ | 394e | +++ |
| 348b | +++ | 355b | +++ | 393b | +++ |
| 356e | +++ | 367b | +++ | 395b | +++ |
| 357b | +++ | 376f | +++ | 396d | +++ |
| 358c | +++ | 391b | +++ | 398d | +++ |
| 359b | +++ | 377c | +++ | 400f | +++ |
| 360c | +++ | 378b | +++ | 401b | +++ |
| 361b | +++ | 370c | +++ | 397b | +++ |
| 362c | +++ | 371b | +++ | 399b | +++ |
| 363b | +++ | 372b | +++ | 402a | ++ |
| 364c | +++ | 374c | +++ | 403a | +++ |
| 365b | +++ | 375b | +++ | 386b | +++ |
| 349d | +++ | 381e | +++ | 389c | +++ |
| 351b | +++ | 382b | +++ | 385c | +++ |
| 353b | +++ | 383c | +++ | 390b | +++ |
| 350h | +++ | 441b | ++ | 388b | +++ |
| 404c | +++ | 440c | +++ | 387c | +++ |
| 405b | +++ | 421c | +++ | 429d | ++ |
| 414c | +++ | 422b | +++ | 430b | ++ |
| 415b | +++ | 423c | +++ | 431c | +++ |
| 417b | +++ | 424b | +++ | 432c | ++ |
| 418c | +++ | 425c | +++ | 433b | +++ |
| 416c | +++ | 426b | +++ | 434c | ++ |
| 436b | +++ | 427c | +++ | 435b | +++ |
| 437f | +++ | 428b | +++ | 446f | +++ |
| 419c | +++ | 445b | ++ | 447b | +++ |
| 420b | +++ | 444d | ++ | 410d | +++ |
| 438c | +++ | 385d | +++ | 411b | +++ |
| 459f | +++ | 385e | +++ | 412c | +++ |
| 459g | ++ | 406c | +++ | 413b | +++ |
| 458f | +++ | 407b | +++ | 448c | +++ |
| 442c | +++ | 408f | +++ | 449b | +++ |
| 443b | +++ | 409b | +++ | 450c | +++ |
| 439b | ++ | 456g | +++ | 451b | +++ |
| 453b | +++ | 457b | +++ | 452c | +++ |
| 454c | ++ | 455b | ++ | | |

### INCORPORATION BY REFERENCE

All of the U.S. patents and U.S. and PCT published patent applications cited herein are hereby incorporated by reference, except for any definitions, subject matter disclaimers or disavowals, and except to the extent that the incorporated material is inconsistent with the express disclosure herein, in which case the language in this disclosure controls.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A compound of formula (I), or a pharmaceutically acceptable salt thereof: 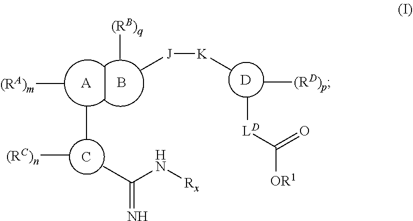
wherein:
ring 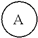
is arylene or heteroarylene;
ring 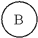
is arylene or heteroarylene;
ring 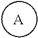
is fused to ring 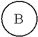
at two and only two adjacent positions;
ring is 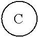
aryl or heteroaryl;
ring 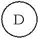
is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or 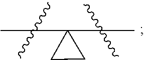 [SMILES: *C1(*)CC1]
K represents a bond, —O—, —NH—, —C(O)—, —CH₂—, —S—, —S(O)—, —SO₂—, —N(alkyl)-, —CH(alkyl)-, or —CH(cycloalkyl)-;
wherein at least one of J and K is a bond, —C(O)—, —CH₂—, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl), —C(O)O(alkyl), —C(O)NH(optionally substituted alkyl), —C(O)NH(cycloalkyl)alkyl), —NHC(O)((alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{x} is H or OH;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

2. The compound of claim 1, wherein:
ring 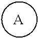
is arylene or heteroarylene;
ring 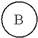
is arylene or heteroarylene;
ring 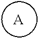
is fused to ring 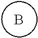
at two and only two adjacent positions;
ring 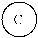
is aryl or heteroaryl;
ring 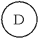
is aryl;
J represents —CH₂— or —C(O)—;
K represents —O—, —NH—, or a bond;
L^{D} represents —CH₂—, —CH(OH)—, or a bond;
R^{A}, independently for each occurrence, represents H, optionally substituted alkoxy, or optionally substituted (heteroaryl)alkoxy;
R^{B}, independently for each occurrence, represents H, oxo, or is optionally substituted alkyl, aryl, cycloalkyl, (cycloalkyl)alkyl, or heterocycloalkyl;
R^{C}, independently for each occurrence, represents H, halo, —OH, or alkoxy;
R^{x} is H or OH;
R^{D}, independently for each occurrence, represents H, or is optionally substituted alkyl or alkoxy;
R¹ represents H; and
m, n, p, and q are each independently 0, 1, or 2.

3. The compound of claim 1 or 2, having the structure of formula (Ia):

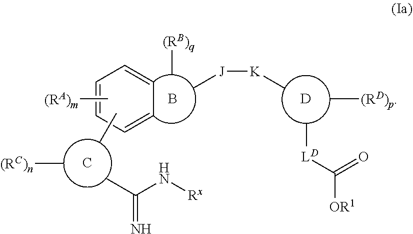

4. The compound of any one of claims 1-3, having the structure of formula (Ia-1):

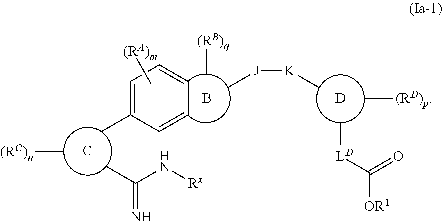

5. The compound of claim 1 or 2, having the structure of formula (Ib):

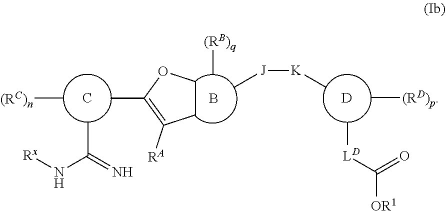

6. The compound of claim 1 or 2, having the structure of formula (Ic):

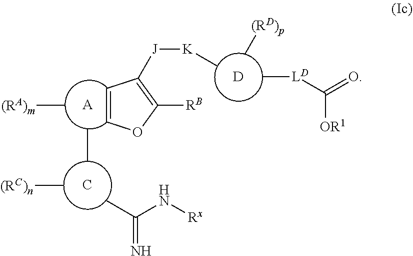

7. The compound of claim 1 or 2, having the structure of formula (Id): 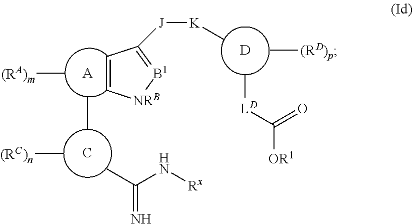
wherein B¹ represents N or CH.

8. The compound of claim 1 or 2, having the structure of formula (Ic):

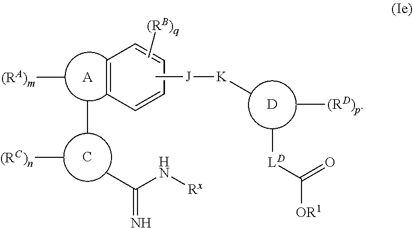

9. The compound of claim 1 or 2, having the structure of formula (If):

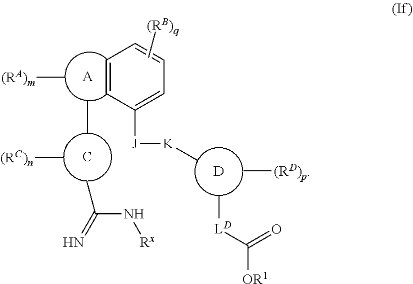

10. The compound of claim 1 or 2, having the structure of formula (Ig):

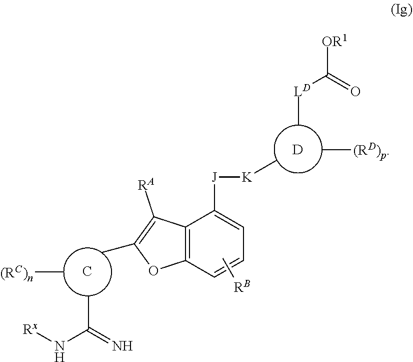

11. The compound of claim 1 or 2, having the structure of formula (Ih):

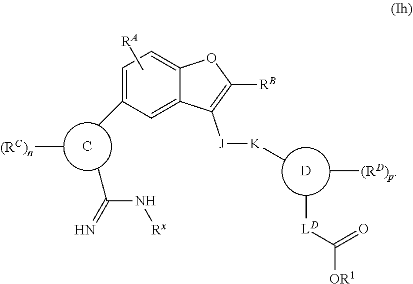

12. The compound of claim 1 or 2, having the structure of formula (Ij):

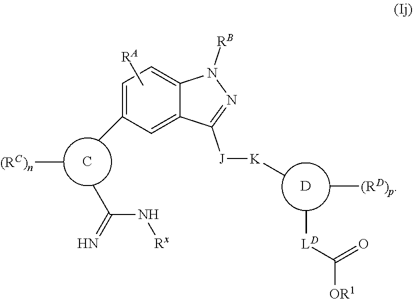

13. The compound of claim 1 or 2, having the structure of formula (Ik):

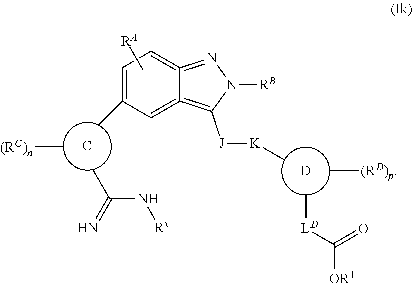

14. The compound of any one of claims 1-13, wherein ring 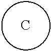
represents a 6-membered aryl or heteroaryl.

15. The compound of any one of claims 1-14, wherein: 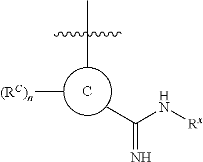
represents 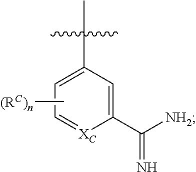
and
X_{C} represents CH or N.

16. The compound of any one of claims 1-15, wherein 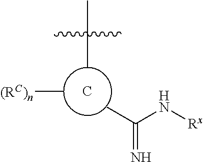
represents

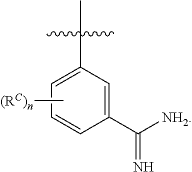

17. The compound of any one of claims 1-16, wherein R^{C} represents H.

18. The compound of any one of claims 1-16, wherein R^{C} represents alkoxy (e.g., methoxy).

19. The compound of any one of claims 1 and 3-18, wherein J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

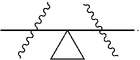 [SMILES: *C1(*)CC1]

20. The compound of any one of claims 1-19, wherein -J-K- represents —CH₂—O— or —C(O)—NH—.

21. The compound of any one of claims 1-20, wherein 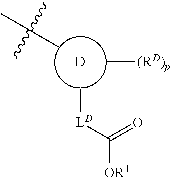
represents

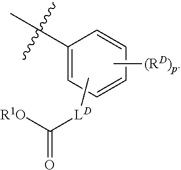

22. The compound of any one of claims 1-21, wherein 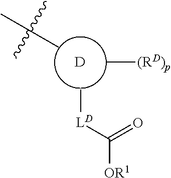
represents

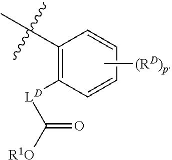

23. The compound of claim 1, wherein ring 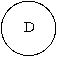
is bicyclic heteroaryl.

24. The compound of claim 23, wherein 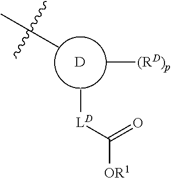
represents

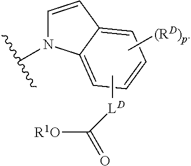

25. The compound of claim 23, wherein 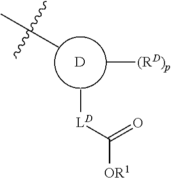
represents

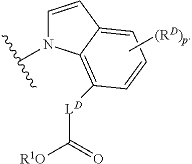

26. The compound of any one of claims 1-25, wherein R¹ is H.

27. The compound of any one of claims 1-26, wherein L^{D} represents —CH₂— or a bond.

28. The compound of any one of claims 1-27, wherein L^{D} represents —CH₂—.

29. The compound of any one of claims 1-28, wherein R^{D}, independently for each occurrence, represents H, alkyl, or alkoxy.

30. The compound of any one of claims 1-29, wherein R^{D} represents H.

31. The compound of any one of claims 1-30, wherein R^{B}, independently for each occurrence, represents H, alkyl, cycloalkyl, (cycloalkyl)alkyl, or heterocycloalkyl.

32. The compound of any one of claims 1-31, wherein R^{B} represents H.

33. The compound of any one of claims 1-31, wherein R^{B}, independently for each occurrence, represents alkyl or cycloalkyl.

34. The compound of any one of claims 1-33, wherein R^{A}, independently for each occurrence, represents H, optionally substituted alkoxy, or optionally substituted (heteroaryl)alkoxy.

35. The compound of any one of claims 1-34, wherein R^{A} represents H.

36. The compound of claim 1, selected from the following table:

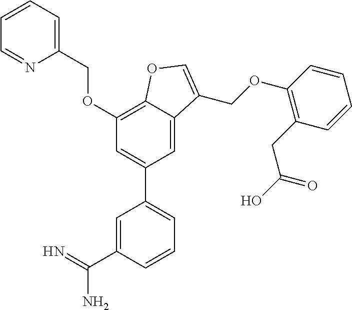 [SMILES: N=C(N)c1cccc(-c2cc(OCc3ccccn3)c3occ(COc4ccccc4CC(=O)O)c3c2)c1]

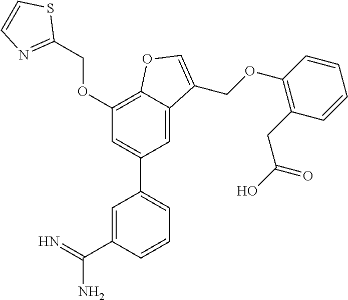 [SMILES: N=C(N)c1cccc(-c2cc(OCc3nccs3)c3occ(COc4ccccc4CC(=O)O)c3c2)c1]

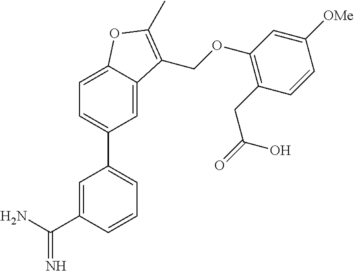 [SMILES: COc1ccc(CC(=O)O)c(OCc2c(C)oc3ccc(-c4cccc(C(=N)N)c4)cc23)c1]

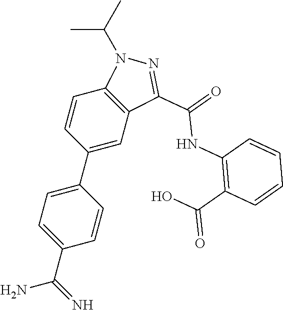 [SMILES: CC(C)n1nc(C(=O)Nc2ccccc2C(=O)O)c2cc(-c3ccc(C(=N)N)cc3)ccc21]

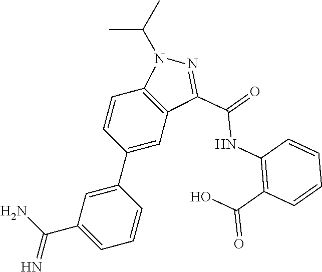 [SMILES: CC(C)n1nc(C(=O)Nc2ccccc2C(=O)O)c2cc(-c3cccc(C(=N)N)c3)ccc21]

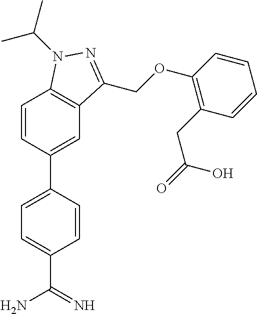 [SMILES: CC(C)n1nc(COc2ccccc2CC(=O)O)c2cc(-c3ccc(C(=N)N)cc3)ccc21]

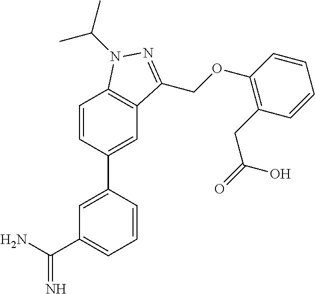 [SMILES: CC(C)n1nc(COc2ccccc2CC(=O)O)c2cc(-c3cccc(C(=N)N)c3)ccc21]

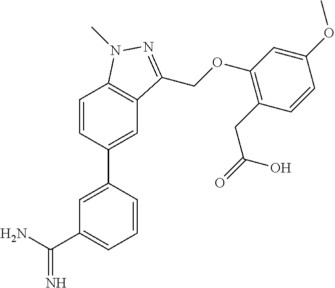 [SMILES: COc1ccc(CC(=O)O)c(OCc2nn(C)c3ccc(-c4cccc(C(=N)N)c4)cc23)c1]

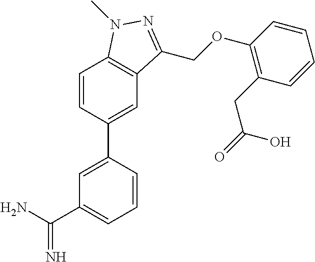 [SMILES: Cn1nc(COc2ccccc2CC(=O)O)c2cc(-c3cccc(C(=N)N)c3)ccc21]

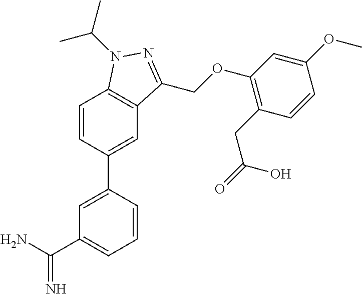 [SMILES: COc1ccc(CC(=O)O)c(OCc2nn(C(C)C)c3ccc(-c4cccc(C(=N)N)c4)cc23)c1]

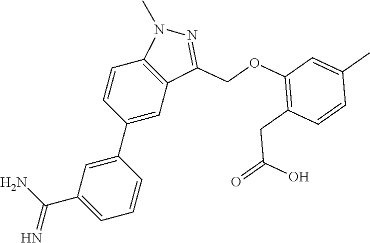 [SMILES: Cc1ccc(CC(=O)O)c(OCc2nn(C)c3ccc(-c4cccc(C(=N)N)c4)cc23)c1]

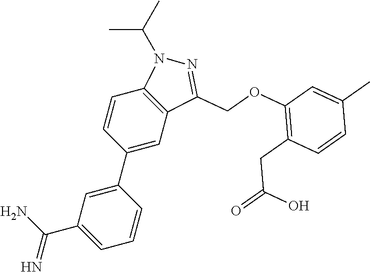 [SMILES: Cc1ccc(CC(=O)O)c(OCc2nn(C(C)C)c3ccc(-c4cccc(C(=N)N)c4)cc23)c1]

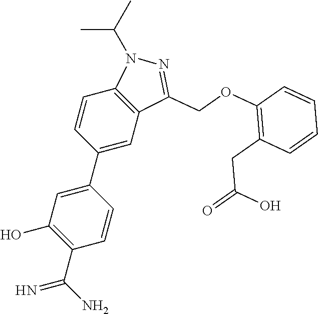 [SMILES: CC(C)n1nc(COc2ccccc2CC(=O)O)c2cc(-c3ccc(C(=N)N)c(O)c3)ccc21]

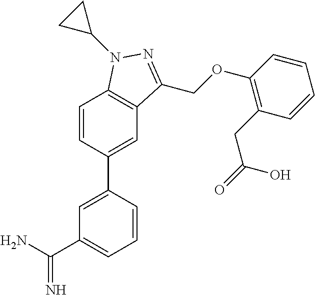 [SMILES: N=C(N)c1cccc(-c2ccc3c(c2)c(COc2ccccc2CC(=O)O)nn3C2CC2)c1]

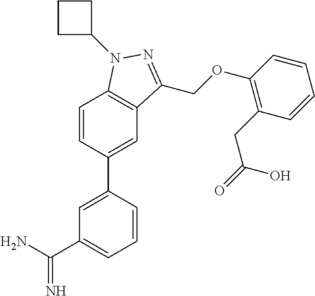 [SMILES: N=C(N)c1cccc(-c2ccc3c(c2)c(COc2ccccc2CC(=O)O)nn3C2CCC2)c1]

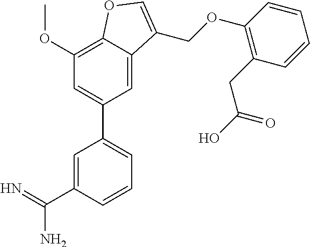 [SMILES: COc1cc(-c2cccc(C(=N)N)c2)cc2c(COc3ccccc3CC(=O)O)coc12]

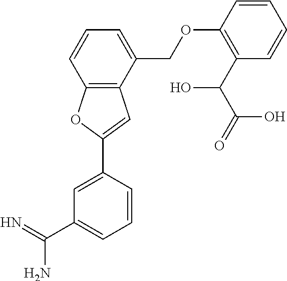 [SMILES: N=C(N)c1cccc(-c2cc3c(COc4ccccc4C(O)C(=O)O)cccc3o2)c1]

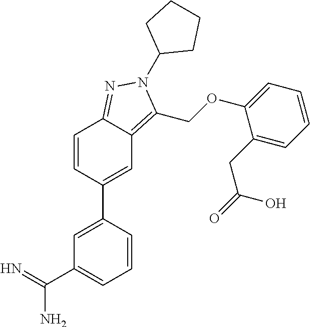 [SMILES: N=C(N)c1cccc(-c2ccc3nn(C4CCCC4)c(COc4ccccc4CC(=O)O)c3c2)c1]

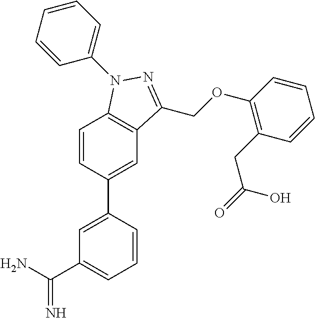 [SMILES: N=C(N)c1cccc(-c2ccc3c(c2)c(COc2ccccc2CC(=O)O)nn3-c2ccccc2)c1]

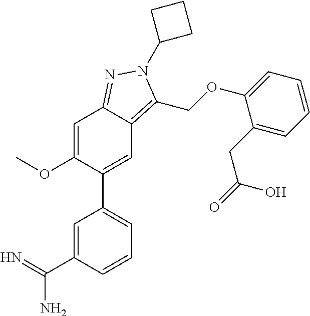 [SMILES: COc1cc2nn(C3CCC3)c(COc3ccccc3CC(=O)O)c2cc1-c1cccc(C(=N)N)c1]

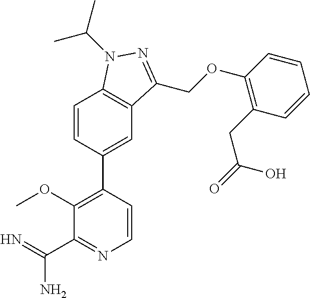 [SMILES: COc1c(-c2ccc3c(c2)c(COc2ccccc2CC(=O)O)nn3C(C)C)ccnc1C(=N)N]

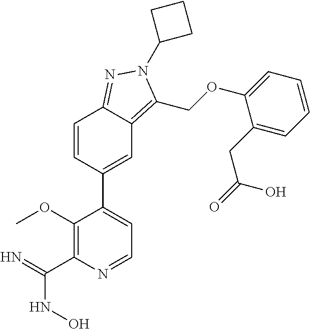 [SMILES: COc1c(-c2ccc3nn(C4CCC4)c(COc4ccccc4CC(=O)O)c3c2)ccnc1C(=N)NO]

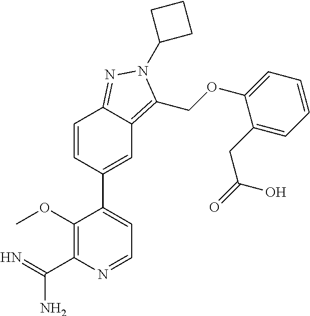 [SMILES: COc1c(-c2ccc3nn(C4CCC4)c(COc4ccccc4CC(=O)O)c3c2)ccnc1C(=N)N]

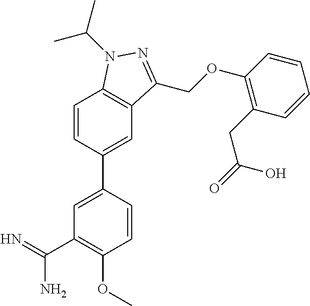 [SMILES: COc1ccc(-c2ccc3c(c2)c(COc2ccccc2CC(=O)O)nn3C(C)C)cc1C(=N)N]

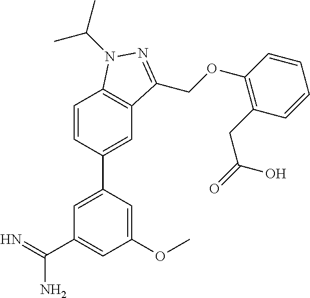 [SMILES: COc1cc(C(=N)N)cc(-c2ccc3c(c2)c(COc2ccccc2CC(=O)O)nn3C(C)C)c1]

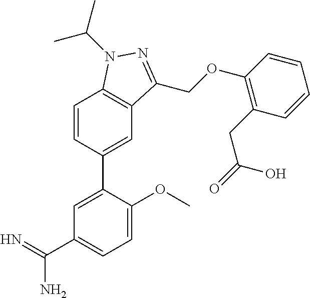 [SMILES: COc1ccc(C(=N)N)cc1-c1ccc2c(c1)c(COc1ccccc1CC(=O)O)nn2C(C)C]

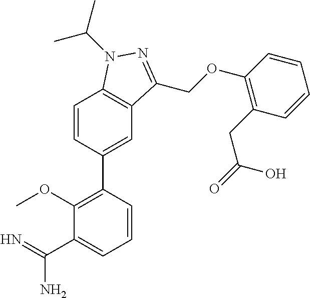 [SMILES: COc1c(C(=N)N)cccc1-c1ccc2c(c1)c(COc1ccccc1CC(=O)O)nn2C(C)C]

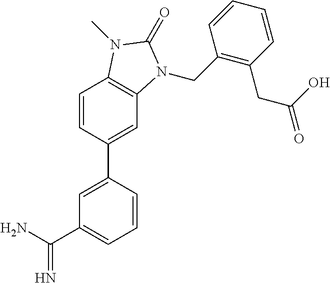 [SMILES: Cn1c(=O)n(Cc2ccccc2CC(=O)O)c2cc(-c3cccc(C(=N)N)c3)ccc21]

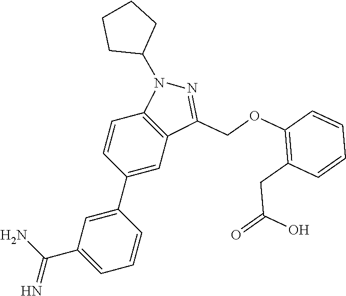 [SMILES: N=C(N)c1cccc(-c2ccc3c(c2)c(COc2ccccc2CC(=O)O)nn3C2CCCC2)c1]

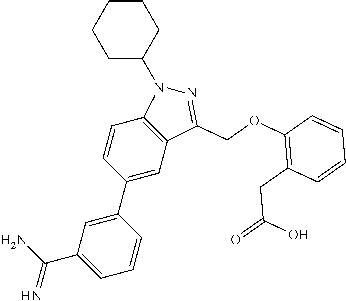 [SMILES: N=C(N)c1cccc(-c2ccc3c(c2)c(COc2ccccc2CC(=O)O)nn3C2CCCCC2)c1]

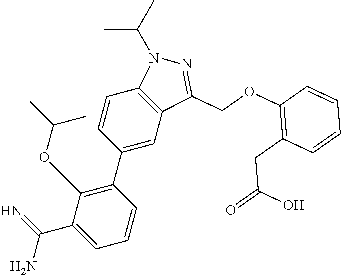 [SMILES: CC(C)Oc1c(C(=N)N)cccc1-c1ccc2c(c1)c(COc1ccccc1CC(=O)O)nn2C(C)C]

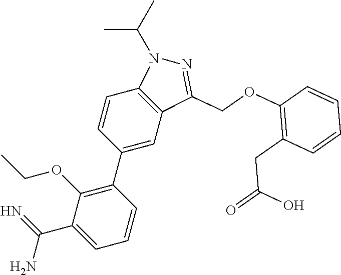 [SMILES: CCOc1c(C(=N)N)cccc1-c1ccc2c(c1)c(COc1ccccc1CC(=O)O)nn2C(C)C]

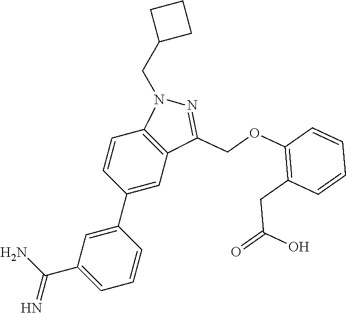 [SMILES: N=C(N)c1cccc(-c2ccc3c(c2)c(COc2ccccc2CC(=O)O)nn3CC2CCC2)c1]

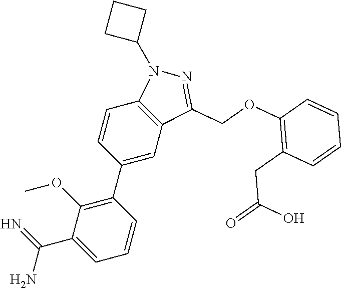 [SMILES: COc1c(C(=N)N)cccc1-c1ccc2c(c1)c(COc1ccccc1CC(=O)O)nn2C1CCC1]

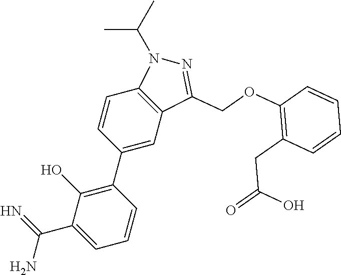 [SMILES: CC(C)n1nc(COc2ccccc2CC(=O)O)c2cc(-c3cccc(C(=N)N)c3O)ccc21]

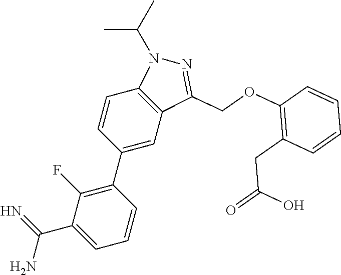 [SMILES: CC(C)n1nc(COc2ccccc2CC(=O)O)c2cc(-c3cccc(C(=N)N)c3F)ccc21]

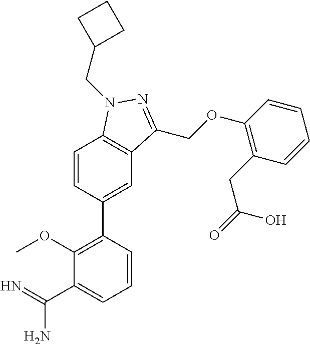 [SMILES: COc1c(C(=N)N)cccc1-c1ccc2c(c1)c(COc1ccccc1CC(=O)O)nn2CC1CCC1]

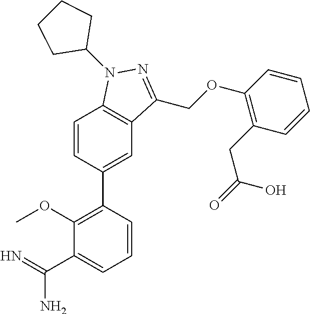 [SMILES: COc1c(C(=N)N)cccc1-c1ccc2c(c1)c(COc1ccccc1CC(=O)O)nn2C1CCCC1]

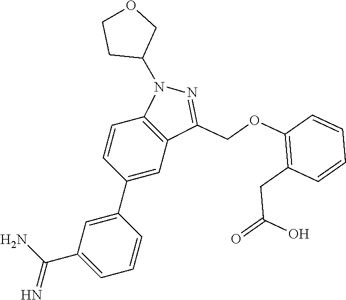 [SMILES: N=C(N)c1cccc(-c2ccc3c(c2)c(COc2ccccc2CC(=O)O)nn3C2CCOC2)c1]

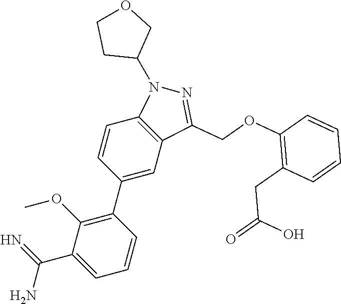 [SMILES: COc1c(C(=N)N)cccc1-c1ccc2c(c1)c(COc1ccccc1CC(=O)O)nn2C1CCOC1]

37. A compound of formula (II), or a pharmaceutically acceptable salt thereof: 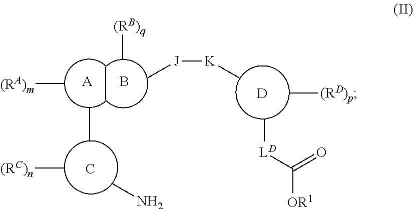
wherein:
ring 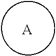
is arylene or heteroarylene;
ring 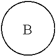
is arylene or heteroarylene;
ring 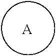
is fused to ring 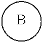
at two and only two adjacent positions;
ring 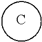
is a bicyclic ring system, where the ring attached to ring 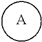
is aryl or heteroaryl;
ring 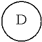
is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or 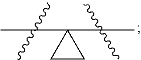 [SMILES: *C1(*)CC1]
K represents a bond, —O—, —NH—, —C(O)—, —CH₂—, —S—, —S(O)—, —SO₂—, —N(alkyl)-, —CH(alkyl)-, or —CH(cycloalkyl)-;
wherein at least one of J and K is a bond, —C(O)—, —CH₂—, —CH₂CH₂—, —CH(alkyl)-, or —CH(aryl)-:
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl), —C(O)O(alkyl), —C(O)NH(optionally substituted alkyl), —C(O)NH((cycloalkyl)alkyl), —NHC(O)((alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

38. The compound of claim 37, wherein:
ring 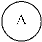
is arylene or heteroarylene;
ring 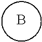
is arylene or heteroarylene;
ring 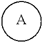
is fused to ring 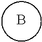
at two and only two adjacent positions;
ring 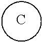
is a bicyclic ring system, where the ring attached to ring 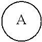
is aryl or heteroaryl;
ring 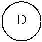
is aryl;
J represents —CH₂— or —C(O)—;
K represents —O— or —NH—;
L^{D} represents —CH₂— or —CH(alkyl)-;
R^{A}, independently for each occurrence, represents H, cyano, alkyl, optionally substituted alkoxy, optionally substituted heteroaryl, optionally substituted aminoalkyl, —C(O)NH₂, or —NH((cycloalkyl)alkyl);
R^{B}, independently for each occurrence, represents H, oxo, alkoxyalkyl, or haloalkyl, or is optionally substituted alkyl, aryl, cycloalkyl, (cycloalkyl)alkyl, spirocycloalkyl, heterocycloalkyl, or (heterocycloalkyl)alkyl;
R^{C}, independently for each occurrence, represents H or halo, or is optionally substituted alkoxy or alkyl;
R^{D}, independently for each occurrence, represents H, halo, cyano, or —C(O)OH, or is optionally substituted alkyl, cycloalkyl, alkoxy, haloalkoxy, or haloalkyl;
R¹ represents H or optionally substituted alkyl.

39. The compound of claim 37 or 38, having the structure of formula (IIa):

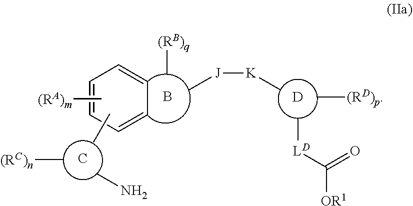

40. The compound of any one of claims 37-39, having the structure of formula (IIa-1):

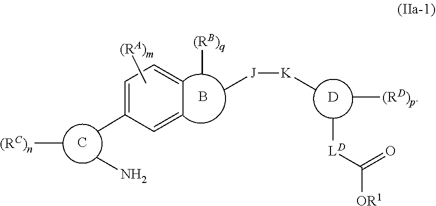

41. The compound of claim 37 or 38, having the structure of formula (IIb):

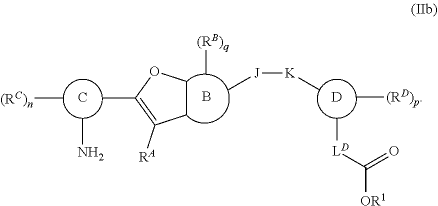

42. The compound of claim 37 or 38, having the structure of formula (IIc):

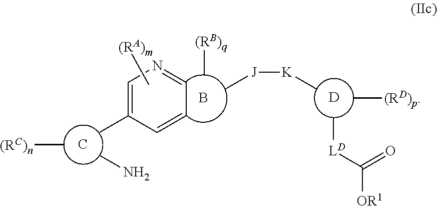

43. The compound of claim 37 or 38, having the structure of formula (IId):

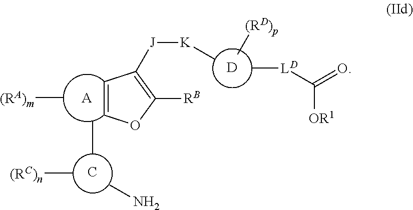

44. The compound of claim 37 or 38, having the structure of formula (IIe): 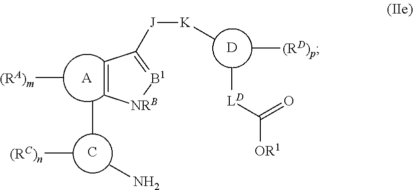
wherein B¹ represents N or CH.

45. The compound of claim 37 or 38, having the structure of formula (IIf):

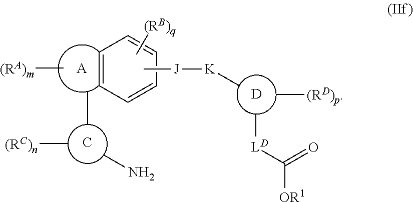

46. The compound of claim 37 or 38, having the structure of formula (IIf-1):

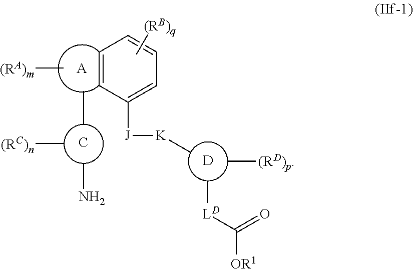

47. The compound of claim 37 or 38, having the structure of formula (Ig):

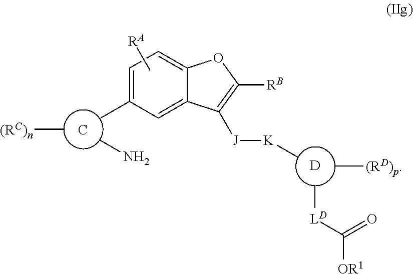

48. The compound of claim 37 or 38, having the structure of formula (IIh):

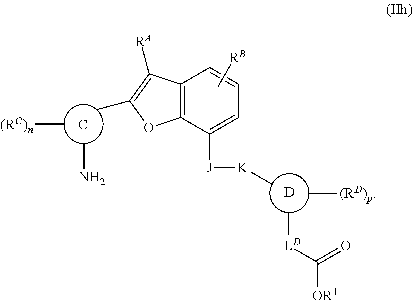

49. The compound of claim 37 or 38, having the structure of formula (IIj):

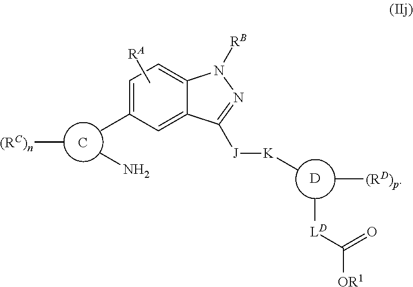

50. The compound of claim 37 or 38, having the structure of formula (IIk):

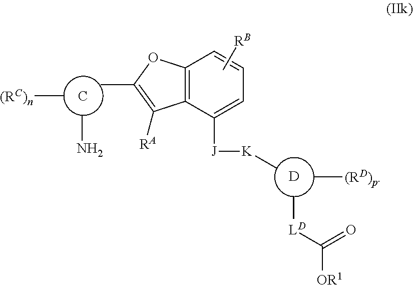

51. The compound of claim 37 or 38, having the structure of formula (IIm):

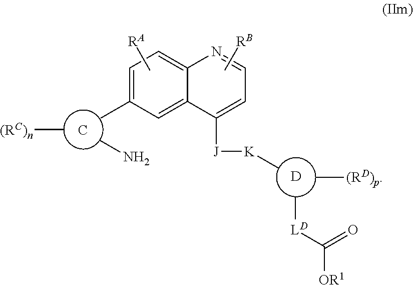

52. The compound of claim 37 or 38, having the structure of formula (IIn):

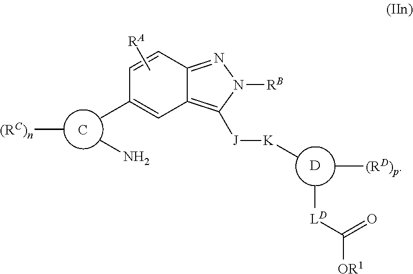

53. The compound of claim 37 or 38, having the structure of formula (IIo): 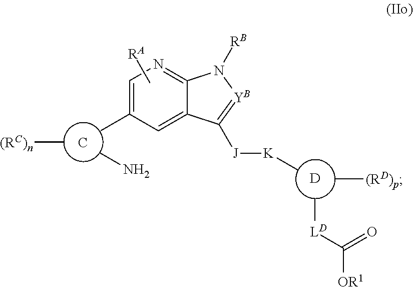
wherein Y^{B} is H or CH.

54. The compound of any one of claims 37-53, wherein ring 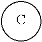
is a bicyclic ring system, where the ring attached to ring 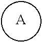
is aryl.

55. The compound of any one of claims 37-53, wherein ring 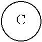
is a bicyclic ring system, where the ring attached to ring 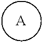
is heteroaryl.

56. The compound of any one of claims 37-54, wherein 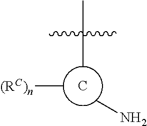
represents 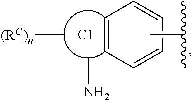
wherein ring 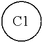
represents aryl, heteroaryl, or heterocycloalkyl.

57. The compound of claim 56, wherein ring 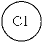
represents heteroaryl.

58. The compound of claim 56 or 57, wherein ring 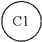
represents a 5- or 6-membered heteroaryl containing at least one nitrogen atom.

59. The compound of any one of claims 37-53 and 56-58, wherein 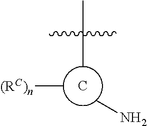
represents

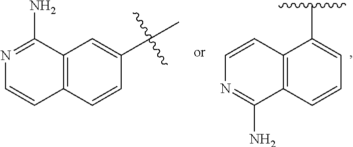 [SMILES: *c1ccc2ccnc(N)c2c1 ; *c1cccc2c(N)nccc12]

60. The compound of any one of claims 37-53 and 55, wherein 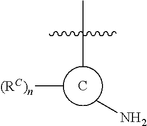
represents 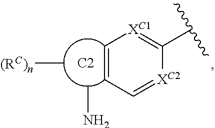
wherein ring 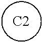
represents aryl, heteroaryl, or heterocycloalkyl; and
X^{C1} and X^{C2} each independently represent CH or N.

61. The compound of claim 60, wherein ring 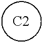
represents aryl or heteroaryl.

62. The compound of claim 60 or 61, wherein 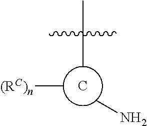
represents

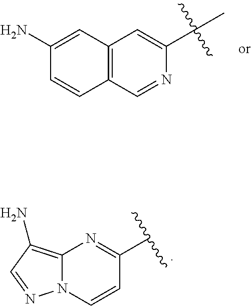 [SMILES: *c1cc2cc(N)ccc2cn1 ; *c1ccn2ncc(N)c2n1]

63. The compound of any one of claims 37-58 and 60-61, wherein R^{C} represents H.

64. The compound of any one of claims 37-58 and 60-61, wherein R^{C}, independently for each occurrence, represents alkoxy or alkyl.

65. The compound of any one of claims 37 and 39-64, wherein J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

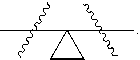 [SMILES: *C1(*)CC1]

66. The compound of any one of claims 37-65, wherein -J-K- represents —CH₂—O— or —C(O)—NH—.

67. The compound of any one of claims 37-66, wherein ring 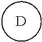
is aryl.

68. The compound of any one of claims 37-67, wherein 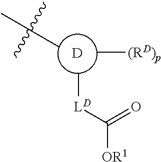
represents

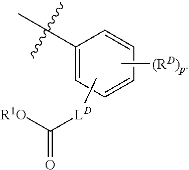

69. The compound of any one of claims 37-68, wherein 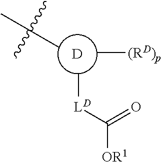
represents

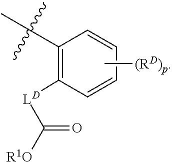

70. The compound of claim 37, wherein ring 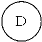
is bicyclic heteroaryl.

71. The compound of claim 70, wherein 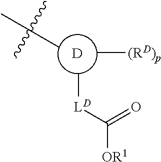
represents

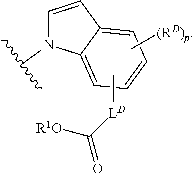

72. The compound of claim 70, wherein 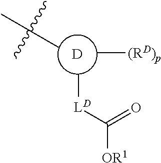
represents

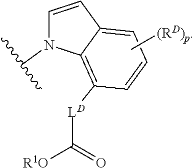

73. The compound of any one of claims 37-72, wherein R¹ is H.

74. The compound of any one of claims 37-73, wherein L^{D} represents —CH₂— or —CH(alkyl)-.

75. The compound of any one of claims 37-74, wherein L^{D} represents —CH₂—.

76. The compound of any one of claims 37-75, wherein R^{D}, independently for each occurrence, represents H, alkyl, or alkoxy.

77. The compound of any one of claims 37-76, wherein R^{D} represents H.

78. The compound of any one of claims 37-77, wherein R^{B}, independently for each occurrence, represents H, alkoxyalkyl, or haloalkyl, or is optionally substituted alkyl, aryl, cycloalkyl, (cycloalkyl)alkyl, heterocycloalkyl, or (heterocycloalkyl)alkyl.

79. The compound of any one of claims 37-78, wherein R^{B}, independently for each occurrence, represents optionally substituted alkyl, cycloalkyl, or heterocycloalkyl, e.g., heterocycloalkyl substituted with alkyl, hydroxyalkyl, alkoxyalkyl.

80. The compound of any one of claims 37-79, wherein R^{A}, independently for each occurrence, represents H, cyano, alkyl, optionally substituted alkoxy, optionally substituted heteroaryl, or —NH((cycloalkyl)alkyl).

81. The compound of any one of claims 37-80, wherein R^{A} represents H.

82. The compound of claim 37, selected from the following table:

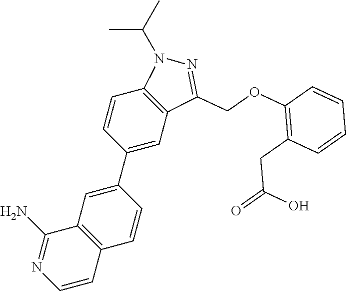 [SMILES: CC(C)n1nc(COc2ccccc2CC(=O)O)c2cc(-c3ccc4ccnc(N)c4c3)ccc21]

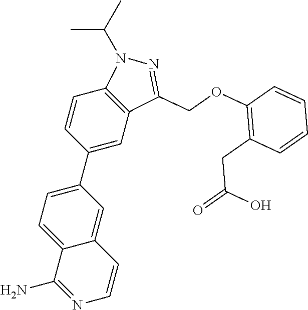 [SMILES: CC(C)n1nc(COc2ccccc2CC(=O)O)c2cc(-c3ccc4c(N)nccc4c3)ccc21]

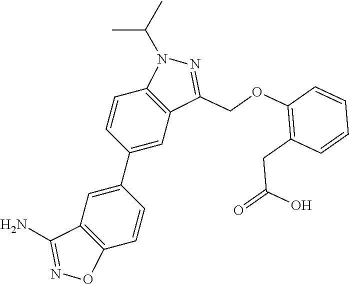 [SMILES: CC(C)n1nc(COc2ccccc2CC(=O)O)c2cc(-c3ccc4onc(N)c4c3)ccc21]

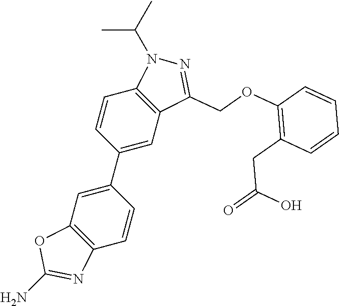 [SMILES: CC(C)n1nc(COc2ccccc2CC(=O)O)c2cc(-c3ccc4nc(N)oc4c3)ccc21]

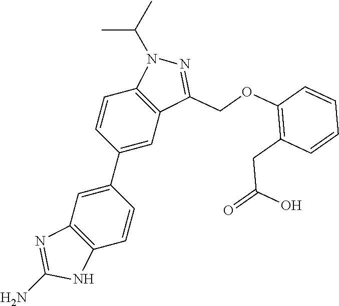 [SMILES: CC(C)n1nc(COc2ccccc2CC(=O)O)c2cc(-c3ccc4[nH]c(N)nc4c3)ccc21]

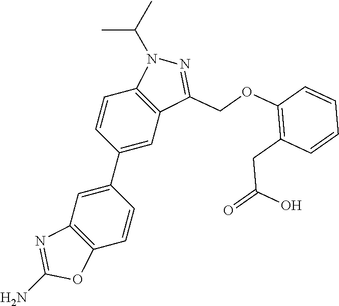 [SMILES: CC(C)n1nc(COc2ccccc2CC(=O)O)c2cc(-c3ccc4oc(N)nc4c3)ccc21]

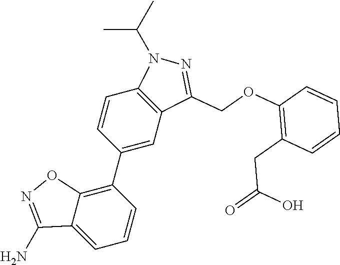 [SMILES: CC(C)n1nc(COc2ccccc2CC(=O)O)c2cc(-c3cccc4c(N)noc34)ccc21]

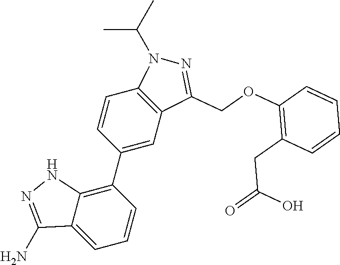 [SMILES: CC(C)n1nc(COc2ccccc2CC(=O)O)c2cc(-c3cccc4c(N)n[nH]c34)ccc21]

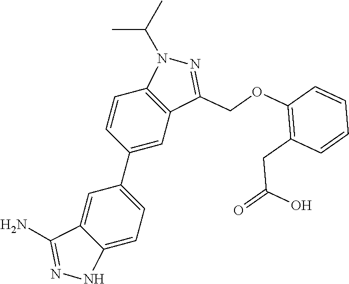 [SMILES: CC(C)n1nc(COc2ccccc2CC(=O)O)c2cc(-c3ccc4[nH]nc(N)c4c3)ccc21]

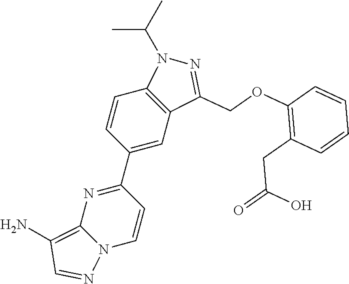 [SMILES: CC(C)n1nc(COc2ccccc2CC(=O)O)c2cc(-c3ccn4ncc(N)c4n3)ccc21]

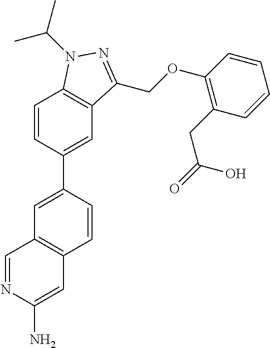 [SMILES: CC(C)n1nc(COc2ccccc2CC(=O)O)c2cc(-c3ccc4cc(N)ncc4c3)ccc21]

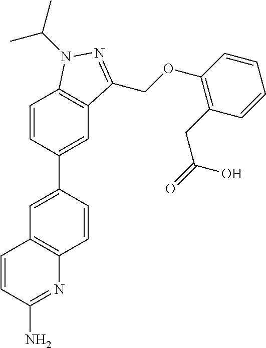 [SMILES: CC(C)n1nc(COc2ccccc2CC(=O)O)c2cc(-c3ccc4nc(N)ccc4c3)ccc21]

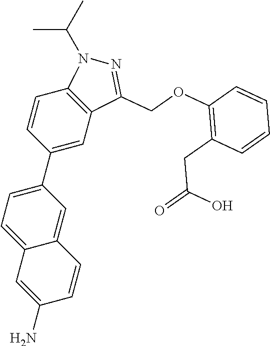 [SMILES: CC(C)n1nc(COc2ccccc2CC(=O)O)c2cc(-c3ccc4cc(N)ccc4c3)ccc21]

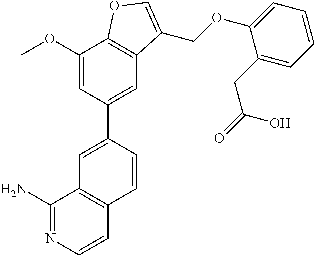 [SMILES: COc1cc(-c2ccc3ccnc(N)c3c2)cc2c(COc3ccccc3CC(=O)O)coc12]

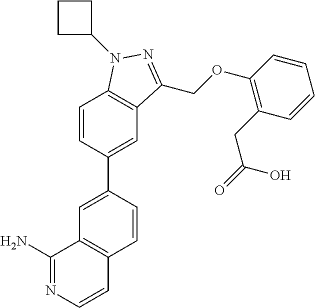 [SMILES: Nc1nccc2ccc(-c3ccc4c(c3)c(COc3ccccc3CC(=O)O)nn4C3CCC3)cc12]

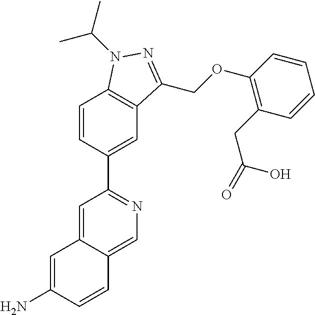 [SMILES: CC(C)n1nc(COc2ccccc2CC(=O)O)c2cc(-c3cc4cc(N)ccc4cn3)ccc21]

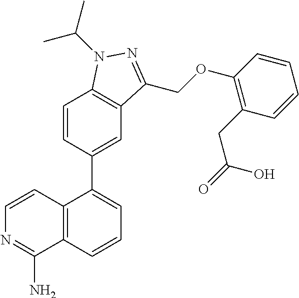 [SMILES: CC(C)n1nc(COc2ccccc2CC(=O)O)c2cc(-c3cccc4c(N)nccc34)ccc21]

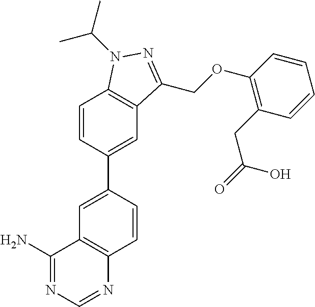 [SMILES: CC(C)n1nc(COc2ccccc2CC(=O)O)c2cc(-c3ccc4ncnc(N)c4c3)ccc21]

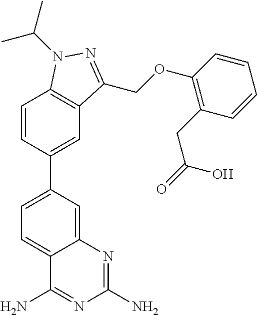 [SMILES: CC(C)n1nc(COc2ccccc2CC(=O)O)c2cc(-c3ccc4c(N)nc(N)nc4c3)ccc21]

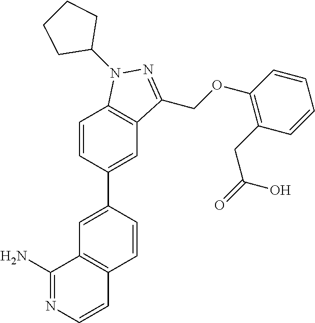 [SMILES: Nc1nccc2ccc(-c3ccc4c(c3)c(COc3ccccc3CC(=O)O)nn4C3CCCC3)cc12]

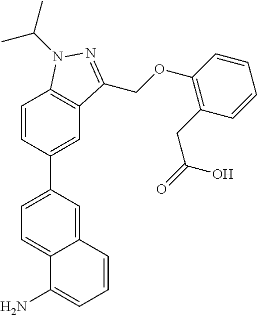 [SMILES: CC(C)n1nc(COc2ccccc2CC(=O)O)c2cc(-c3ccc4c(N)cccc4c3)ccc21]

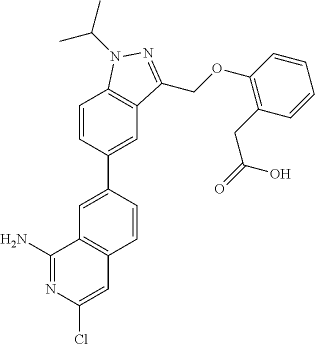 [SMILES: CC(C)n1nc(COc2ccccc2CC(=O)O)c2cc(-c3ccc4cc(Cl)nc(N)c4c3)ccc21]

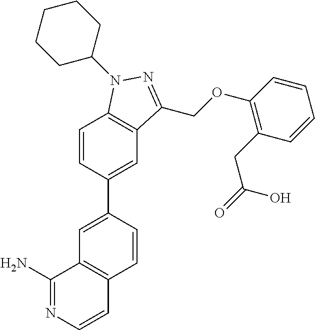 [SMILES: Nc1nccc2ccc(-c3ccc4c(c3)c(COc3ccccc3CC(=O)O)nn4C3CCCCC3)cc12]

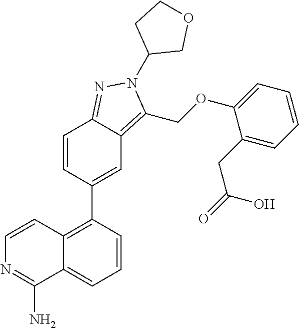 [SMILES: Nc1nccc2c(-c3ccc4nn(C5CCOC5)c(COc5ccccc5CC(=O)O)c4c3)cccc12]

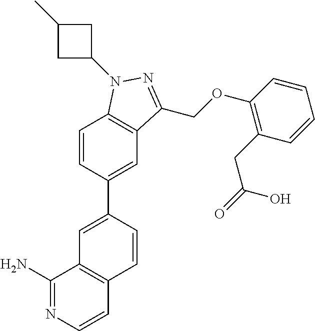 [SMILES: CC1CC(n2nc(COc3ccccc3CC(=O)O)c3cc(-c4ccc5ccnc(N)c5c4)ccc32)C1]

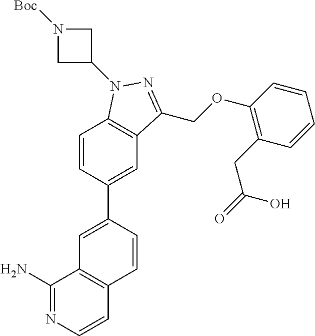 [SMILES: CC(C)(C)OC(=O)N1CC(n2nc(COc3ccccc3CC(=O)O)c3cc(-c4ccc5ccnc(N)c5c4)ccc32)C1]

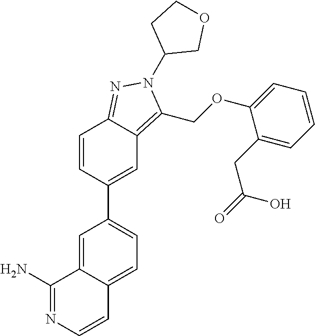 [SMILES: Nc1nccc2ccc(-c3ccc4nn(C5CCOC5)c(COc5ccccc5CC(=O)O)c4c3)cc12]

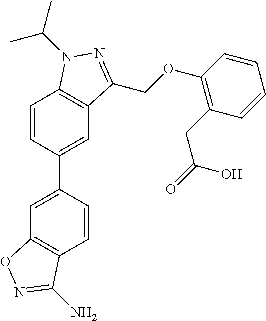 [SMILES: CC(C)n1nc(COc2ccccc2CC(=O)O)c2cc(-c3ccc4c(N)noc4c3)ccc21]

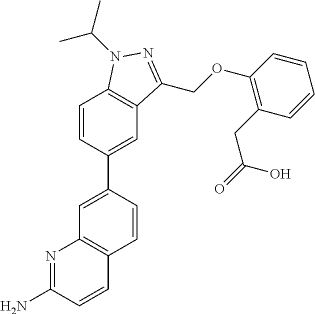 [SMILES: CC(C)n1nc(COc2ccccc2CC(=O)O)c2cc(-c3ccc4ccc(N)nc4c3)ccc21]

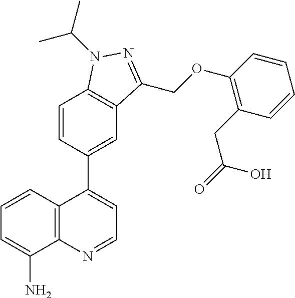 [SMILES: CC(C)n1nc(COc2ccccc2CC(=O)O)c2cc(-c3ccnc4c(N)cccc34)ccc21]

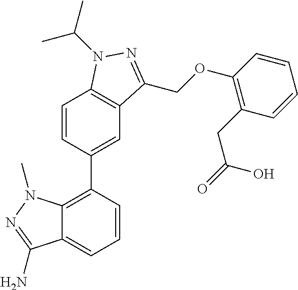 [SMILES: CC(C)n1nc(COc2ccccc2CC(=O)O)c2cc(-c3cccc4c(N)nn(C)c34)ccc21]

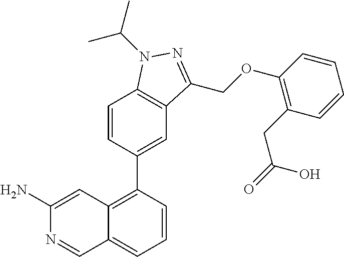 [SMILES: CC(C)n1nc(COc2ccccc2CC(=O)O)c2cc(-c3cccc4cnc(N)cc34)ccc21]

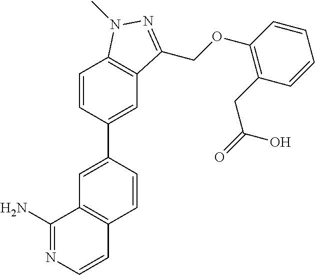 [SMILES: Cn1nc(COc2ccccc2CC(=O)O)c2cc(-c3ccc4ccnc(N)c4c3)ccc21]

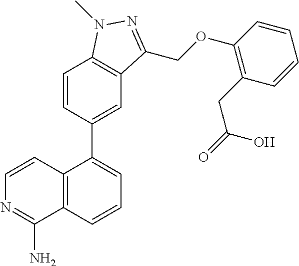 [SMILES: Cn1nc(COc2ccccc2CC(=O)O)c2cc(-c3cccc4c(N)nccc34)ccc21]

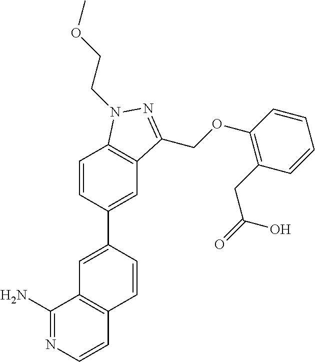 [SMILES: COCCn1nc(COc2ccccc2CC(=O)O)c2cc(-c3ccc4ccnc(N)c4c3)ccc21]

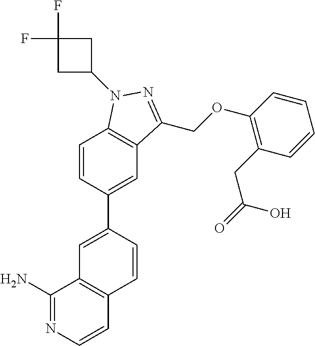 [SMILES: Nc1nccc2ccc(-c3ccc4c(c3)c(COc3ccccc3CC(=O)O)nn4C3CC(F)(F)C3)cc12]

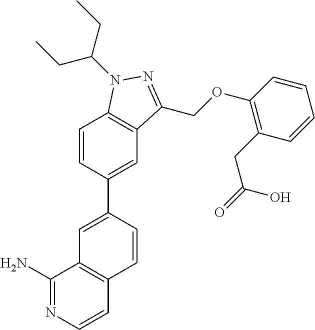 [SMILES: CCC(CC)n1nc(COc2ccccc2CC(=O)O)c2cc(-c3ccc4ccnc(N)c4c3)ccc21]

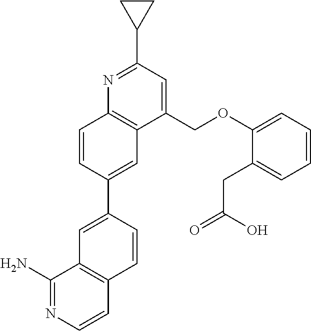 [SMILES: Nc1nccc2ccc(-c3ccc4nc(C5CC5)cc(COc5ccccc5CC(=O)O)c4c3)cc12]

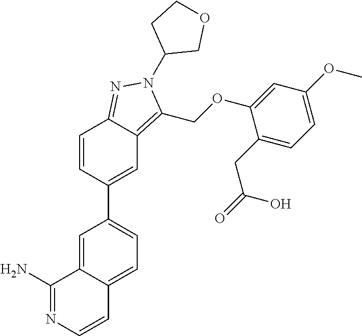 [SMILES: COc1ccc(CC(=O)O)c(OCc2c3cc(-c4ccc5ccnc(N)c5c4)ccc3nn2C2CCOC2)c1]

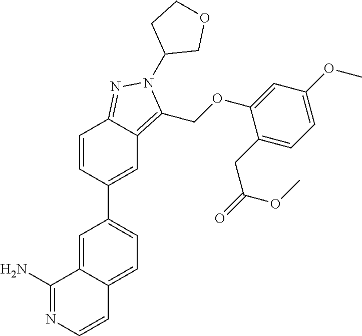 [SMILES: COC(=O)Cc1ccc(OC)cc1OCc1c2cc(-c3ccc4ccnc(N)c4c3)ccc2nn1C1CCOC1]

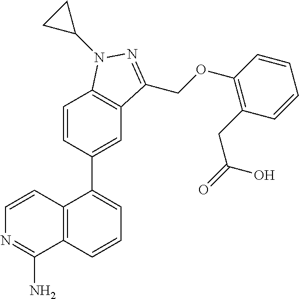 [SMILES: Nc1nccc2c(-c3ccc4c(c3)c(COc3ccccc3CC(=O)O)nn4C3CC3)cccc12]

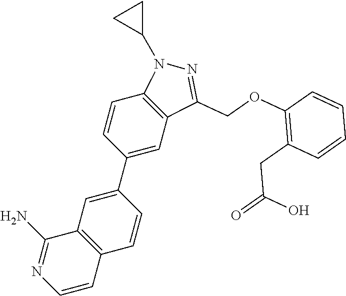 [SMILES: Nc1nccc2ccc(-c3ccc4c(c3)c(COc3ccccc3CC(=O)O)nn4C3CC3)cc12]

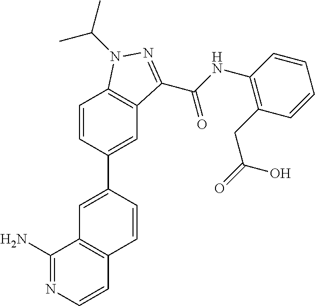 [SMILES: CC(C)n1nc(C(=O)Nc2ccccc2CC(=O)O)c2cc(-c3ccc4ccnc(N)c4c3)ccc21]

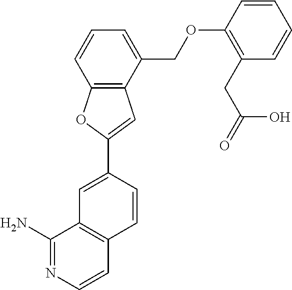 [SMILES: Nc1nccc2ccc(-c3cc4c(COc5ccccc5CC(=O)O)cccc4o3)cc12]

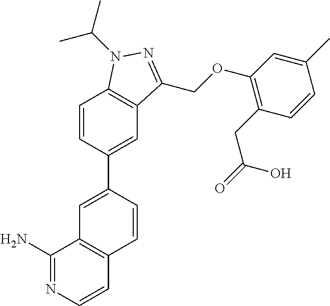 [SMILES: Cc1ccc(CC(=O)O)c(OCc2nn(C(C)C)c3ccc(-c4ccc5ccnc(N)c5c4)cc23)c1]

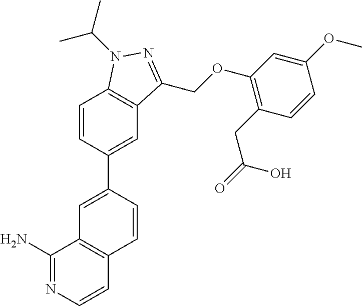 [SMILES: COc1ccc(CC(=O)O)c(OCc2nn(C(C)C)c3ccc(-c4ccc5ccnc(N)c5c4)cc23)c1]

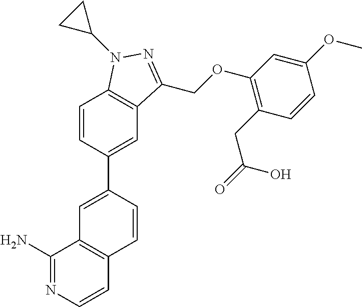 [SMILES: COc1ccc(CC(=O)O)c(OCc2nn(C3CC3)c3ccc(-c4ccc5ccnc(N)c5c4)cc23)c1]

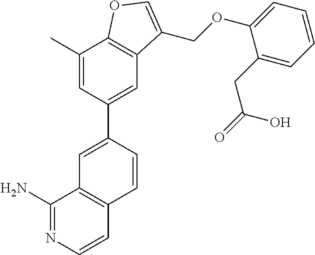 [SMILES: Cc1cc(-c2ccc3ccnc(N)c3c2)cc2c(COc3ccccc3CC(=O)O)coc12]

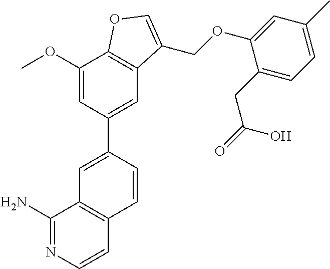 [SMILES: COc1cc(-c2ccc3ccnc(N)c3c2)cc2c(COc3cc(C)ccc3CC(=O)O)coc12]

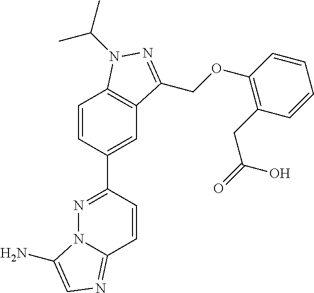 [SMILES: CC(C)n1nc(COc2ccccc2CC(=O)O)c2cc(-c3ccc4ncc(N)n4n3)ccc21]

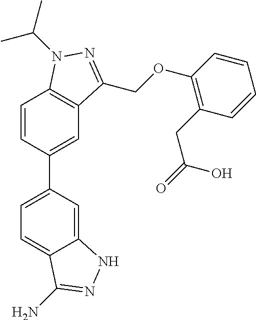 [SMILES: CC(C)n1nc(COc2ccccc2CC(=O)O)c2cc(-c3ccc4c(N)n[nH]c4c3)ccc21]

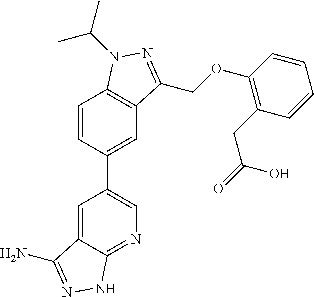 [SMILES: CC(C)n1nc(COc2ccccc2CC(=O)O)c2cc(-c3cnc4[nH]nc(N)c4c3)ccc21]

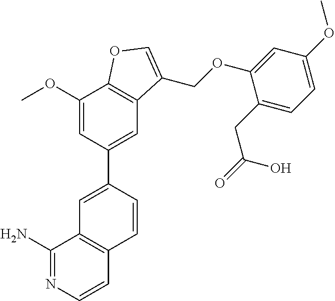 [SMILES: COc1ccc(CC(=O)O)c(OCc2coc3c(OC)cc(-c4ccc5ccnc(N)c5c4)cc23)c1]

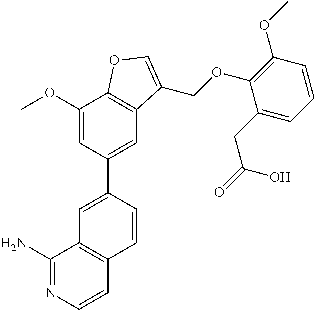 [SMILES: COc1cccc(CC(=O)O)c1OCc1coc2c(OC)cc(-c3ccc4ccnc(N)c4c3)cc12]

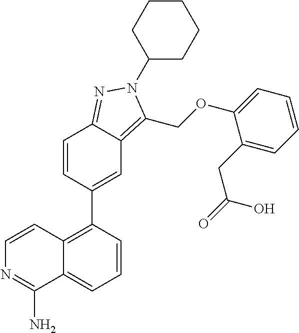 [SMILES: Nc1nccc2c(-c3ccc4nn(C5CCCCC5)c(COc5ccccc5CC(=O)O)c4c3)cccc12]

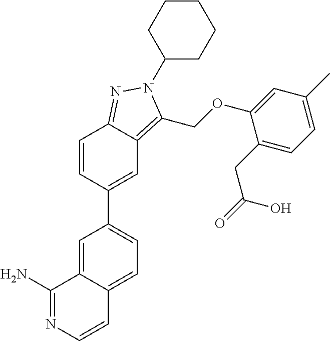 [SMILES: Cc1ccc(CC(=O)O)c(OCc2c3cc(-c4ccc5ccnc(N)c5c4)ccc3nn2C2CCCCC2)c1]

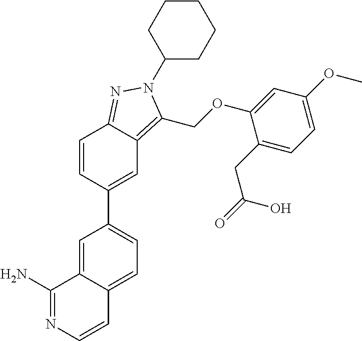 [SMILES: COc1ccc(CC(=O)O)c(OCc2c3cc(-c4ccc5ccnc(N)c5c4)ccc3nn2C2CCCCC2)c1]

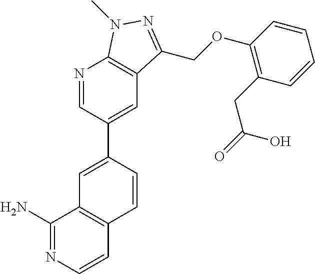 [SMILES: Cn1nc(COc2ccccc2CC(=O)O)c2cc(-c3ccc4ccnc(N)c4c3)cnc21]

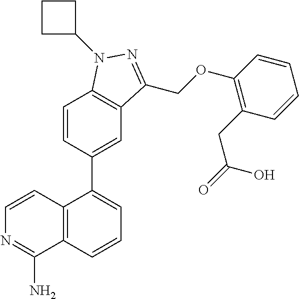 [SMILES: Nc1nccc2c(-c3ccc4c(c3)c(COc3ccccc3CC(=O)O)nn4C3CCC3)cccc12]

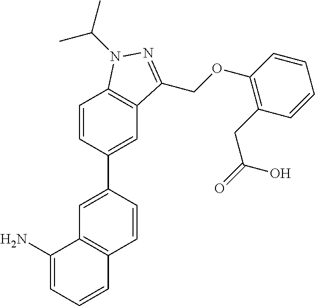 [SMILES: CC(C)n1nc(COc2ccccc2CC(=O)O)c2cc(-c3ccc4cccc(N)c4c3)ccc21]

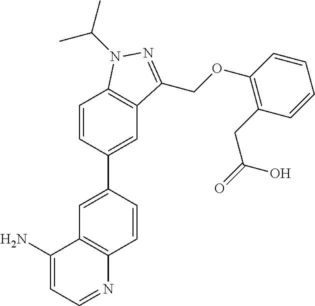 [SMILES: CC(C)n1nc(COc2ccccc2CC(=O)O)c2cc(-c3ccc4nccc(N)c4c3)ccc21]

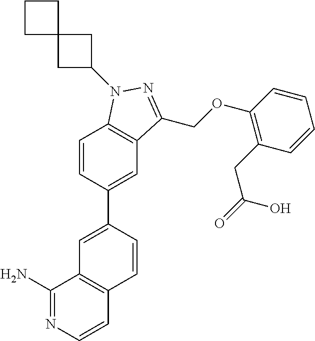 [SMILES: Nc1nccc2ccc(-c3ccc4c(c3)c(COc3ccccc3CC(=O)O)nn4C3CC4(CCC4)C3)cc12]

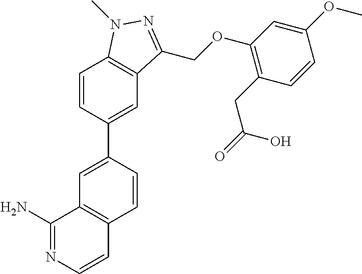 [SMILES: COc1ccc(CC(=O)O)c(OCc2nn(C)c3ccc(-c4ccc5ccnc(N)c5c4)cc23)c1]

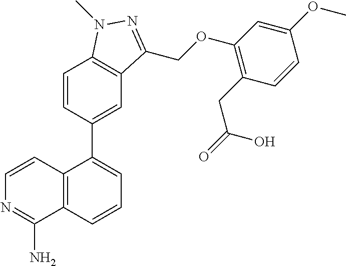 [SMILES: COc1ccc(CC(=O)O)c(OCc2nn(C)c3ccc(-c4cccc5c(N)nccc45)cc23)c1]

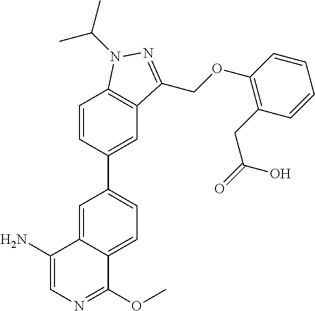 [SMILES: COc1ncc(N)c2cc(-c3ccc4c(c3)c(COc3ccccc3CC(=O)O)nn4C(C)C)ccc12]

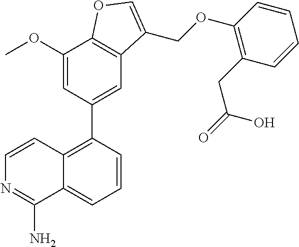 [SMILES: COc1cc(-c2cccc3c(N)nccc23)cc2c(COc3ccccc3CC(=O)O)coc12]

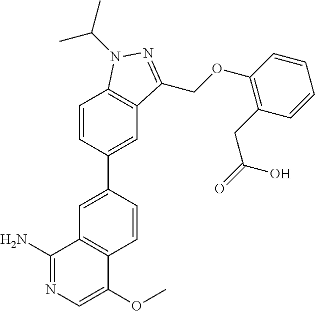 [SMILES: COc1cnc(N)c2cc(-c3ccc4c(c3)c(COc3ccccc3CC(=O)O)nn4C(C)C)ccc12]

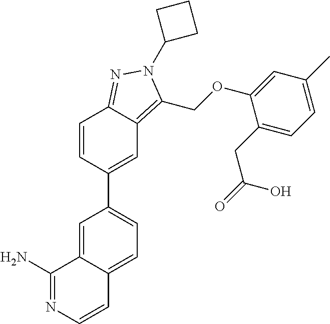 [SMILES: Cc1ccc(CC(=O)O)c(OCc2c3cc(-c4ccc5ccnc(N)c5c4)ccc3nn2C2CCC2)c1]

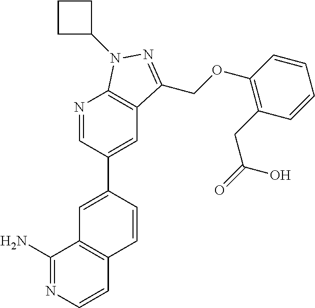 [SMILES: Nc1nccc2ccc(-c3cnc4c(c3)c(COc3ccccc3CC(=O)O)nn4C3CCC3)cc12]

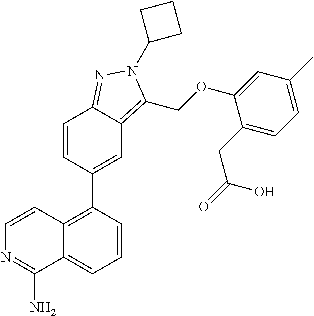 [SMILES: Cc1ccc(CC(=O)O)c(OCc2c3cc(-c4cccc5c(N)nccc45)ccc3nn2C2CCC2)c1]

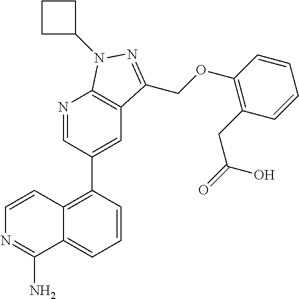 [SMILES: Nc1nccc2c(-c3cnc4c(c3)c(COc3ccccc3CC(=O)O)nn4C3CCC3)cccc12]

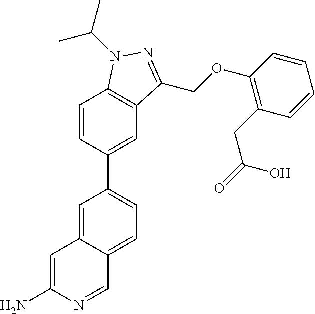 [SMILES: CC(C)n1nc(COc2ccccc2CC(=O)O)c2cc(-c3ccc4cnc(N)cc4c3)ccc21]

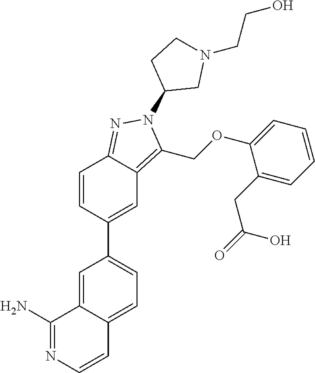 [SMILES: Nc1nccc2ccc(-c3ccc4nn([C@H]5CCN(CCO)C5)c(COc5ccccc5CC(=O)O)c4c3)cc12]

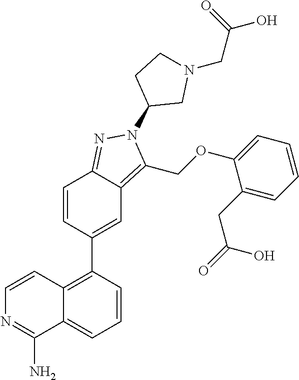 [SMILES: Nc1nccc2c(-c3ccc4nn([C@H]5CCN(CC(=O)O)C5)c(COc5ccccc5CC(=O)O)c4c3)cccc12]

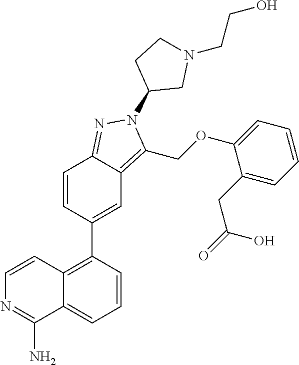 [SMILES: Nc1nccc2c(-c3ccc4nn([C@H]5CCN(CCO)C5)c(COc5ccccc5CC(=O)O)c4c3)cccc12]

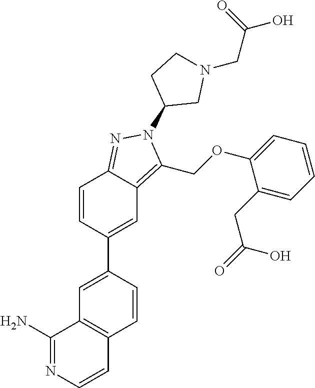 [SMILES: Nc1nccc2ccc(-c3ccc4nn([C@H]5CCN(CC(=O)O)C5)c(COc5ccccc5CC(=O)O)c4c3)cc12]

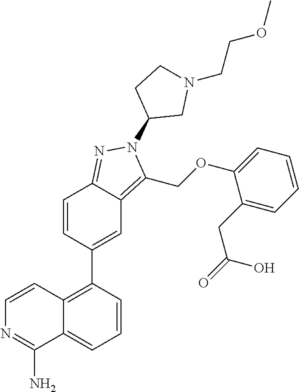 [SMILES: COCCN1CC[C@H](n2nc3ccc(-c4cccc5c(N)nccc45)cc3c2COc2ccccc2CC(=O)O)C1]

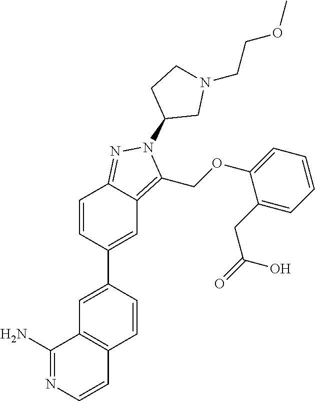 [SMILES: COCCN1CC[C@H](n2nc3ccc(-c4ccc5ccnc(N)c5c4)cc3c2COc2ccccc2CC(=O)O)C1]

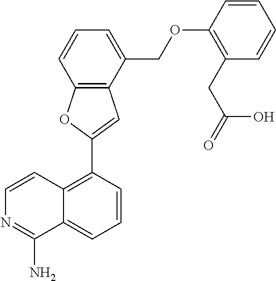 [SMILES: Nc1nccc2c(-c3cc4c(COc5ccccc5CC(=O)O)cccc4o3)cccc12]

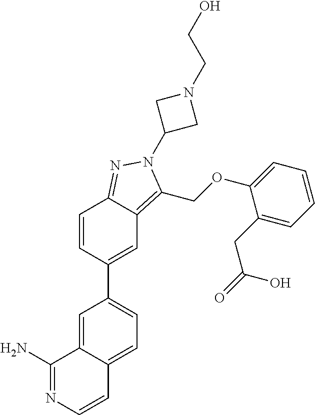 [SMILES: Nc1nccc2ccc(-c3ccc4nn(C5CN(CCO)C5)c(COc5ccccc5CC(=O)O)c4c3)cc12]

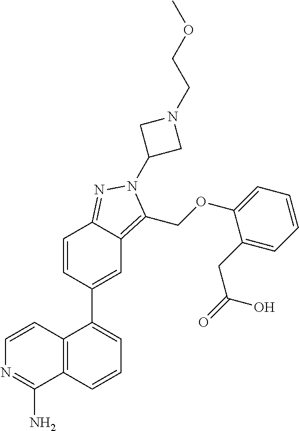 [SMILES: COCCN1CC(n2nc3ccc(-c4cccc5c(N)nccc45)cc3c2COc2ccccc2CC(=O)O)C1]

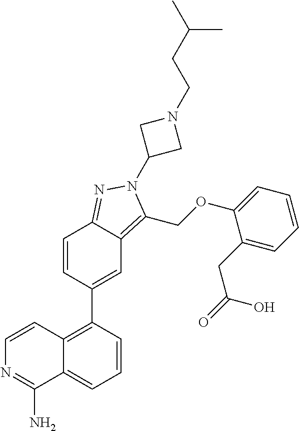 [SMILES: CC(C)CCN1CC(n2nc3ccc(-c4cccc5c(N)nccc45)cc3c2COc2ccccc2CC(=O)O)C1]

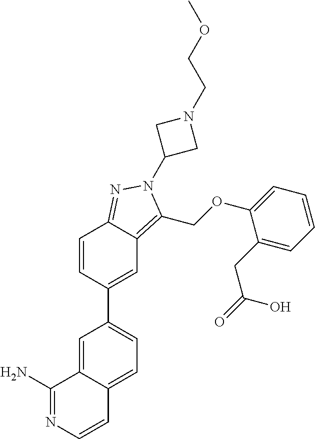 [SMILES: COCCN1CC(n2nc3ccc(-c4ccc5ccnc(N)c5c4)cc3c2COc2ccccc2CC(=O)O)C1]

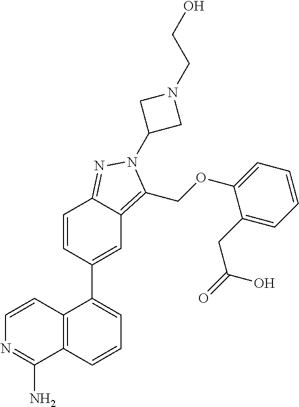 [SMILES: Nc1nccc2c(-c3ccc4nn(C5CN(CCO)C5)c(COc5ccccc5CC(=O)O)c4c3)cccc12]

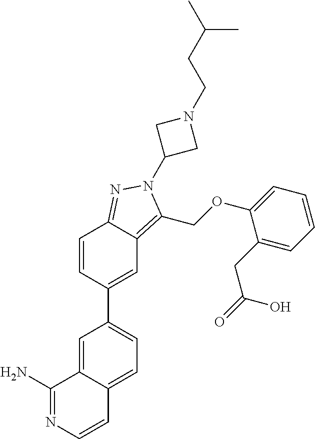 [SMILES: CC(C)CCN1CC(n2nc3ccc(-c4ccc5ccnc(N)c5c4)cc3c2COc2ccccc2CC(=O)O)C1]

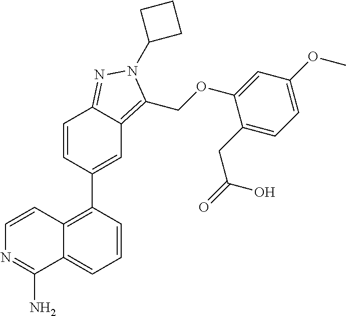 [SMILES: COc1ccc(CC(=O)O)c(OCc2c3cc(-c4cccc5c(N)nccc45)ccc3nn2C2CCC2)c1]

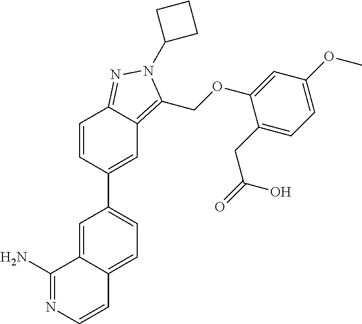 [SMILES: COc1ccc(CC(=O)O)c(OCc2c3cc(-c4ccc5ccnc(N)c5c4)ccc3nn2C2CCC2)c1]

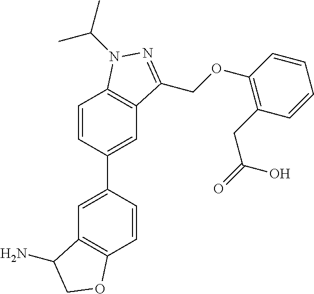 [SMILES: CC(C)n1nc(COc2ccccc2CC(=O)O)c2cc(-c3ccc4c(c3)C(N)CO4)ccc21]

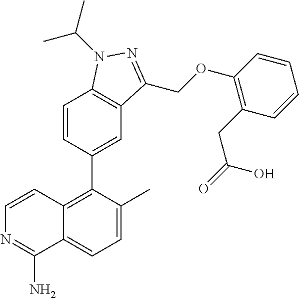 [SMILES: Cc1ccc2c(N)nccc2c1-c1ccc2c(c1)c(COc1ccccc1CC(=O)O)nn2C(C)C]

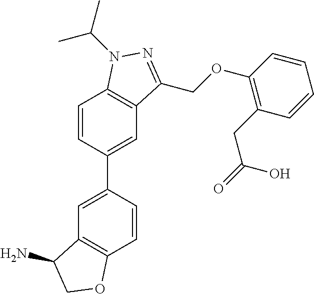 [SMILES: CC(C)n1nc(COc2ccccc2CC(=O)O)c2cc(-c3ccc4c(c3)[C@H](N)CO4)ccc21]

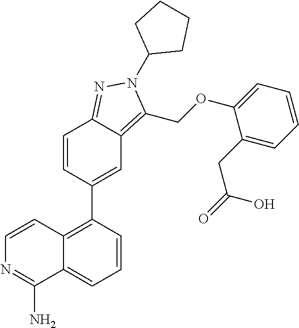 [SMILES: Nc1nccc2c(-c3ccc4nn(C5CCCC5)c(COc5ccccc5CC(=O)O)c4c3)cccc12]

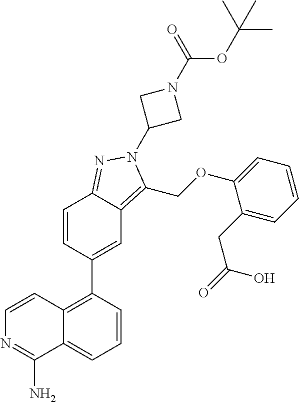 [SMILES: CC(C)(C)OC(=O)N1CC(n2nc3ccc(-c4cccc5c(N)nccc45)cc3c2COc2ccccc2CC(=O)O)C1]

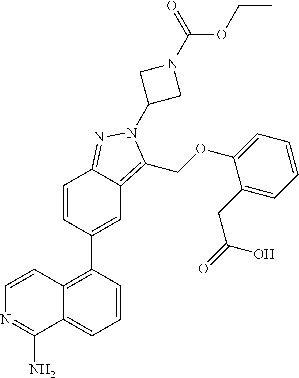 [SMILES: CCOC(=O)N1CC(n2nc3ccc(-c4cccc5c(N)nccc45)cc3c2COc2ccccc2CC(=O)O)C1]

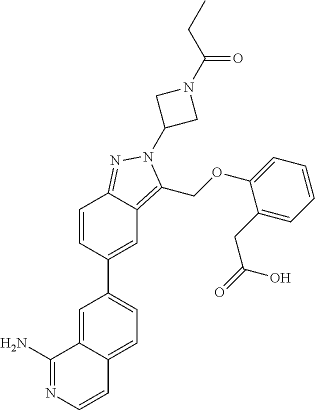 [SMILES: CCC(=O)N1CC(n2nc3ccc(-c4ccc5ccnc(N)c5c4)cc3c2COc2ccccc2CC(=O)O)C1]

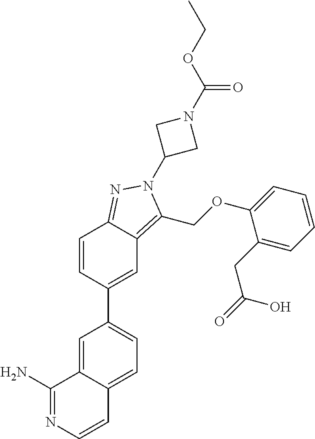 [SMILES: CCOC(=O)N1CC(n2nc3ccc(-c4ccc5ccnc(N)c5c4)cc3c2COc2ccccc2CC(=O)O)C1]

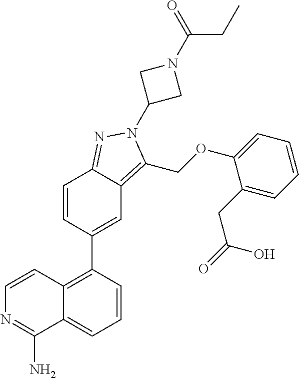 [SMILES: CCC(=O)N1CC(n2nc3ccc(-c4cccc5c(N)nccc45)cc3c2COc2ccccc2CC(=O)O)C1]

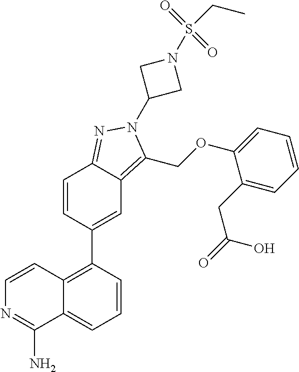 [SMILES: CCS(=O)(=O)N1CC(n2nc3ccc(-c4cccc5c(N)nccc45)cc3c2COc2ccccc2CC(=O)O)C1]

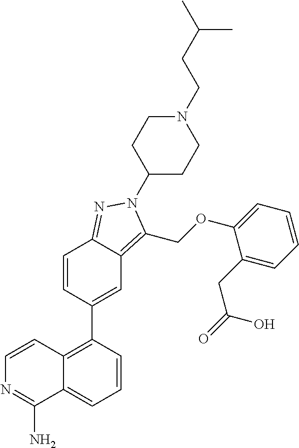 [SMILES: CC(C)CCN1CCC(n2nc3ccc(-c4cccc5c(N)nccc45)cc3c2COc2ccccc2CC(=O)O)CC1]

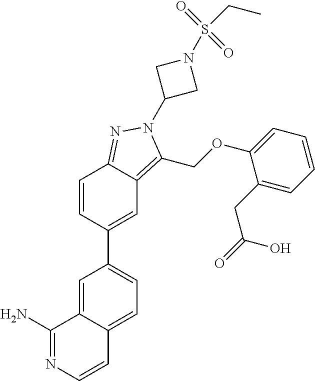 [SMILES: CCS(=O)(=O)N1CC(n2nc3ccc(-c4ccc5ccnc(N)c5c4)cc3c2COc2ccccc2CC(=O)O)C1]

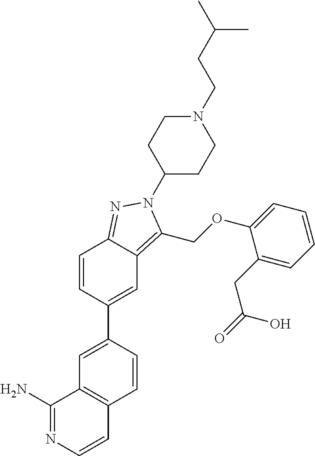 [SMILES: CC(C)CCN1CCC(n2nc3ccc(-c4ccc5ccnc(N)c5c4)cc3c2COc2ccccc2CC(=O)O)CC1]

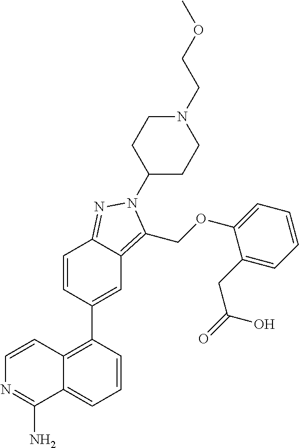 [SMILES: COCCN1CCC(n2nc3ccc(-c4cccc5c(N)nccc45)cc3c2COc2ccccc2CC(=O)O)CC1]

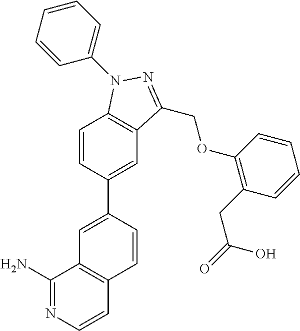 [SMILES: Nc1nccc2ccc(-c3ccc4c(c3)c(COc3ccccc3CC(=O)O)nn4-c3ccccc3)cc12]

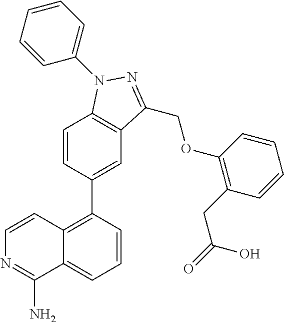 [SMILES: Nc1nccc2c(-c3ccc4c(c3)c(COc3ccccc3CC(=O)O)nn4-c3ccccc3)cccc12]

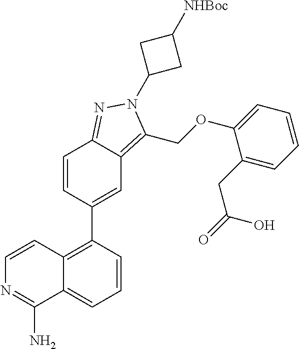 [SMILES: CC(C)(C)OC(=O)NC1CC(n2nc3ccc(-c4cccc5c(N)nccc45)cc3c2COc2ccccc2CC(=O)O)C1]

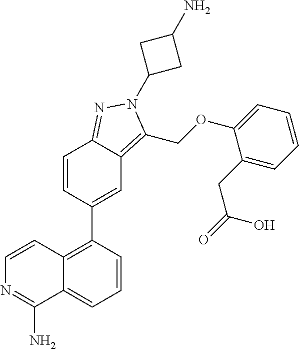 [SMILES: Nc1nccc2c(-c3ccc4nn(C5CC(N)C5)c(COc5ccccc5CC(=O)O)c4c3)cccc12]

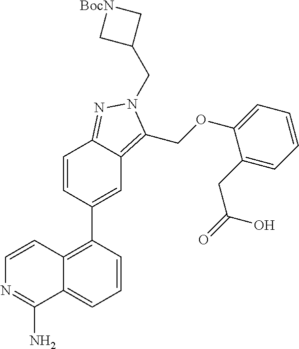 [SMILES: CC(C)(C)OC(=O)N1CC(Cn2nc3ccc(-c4cccc5c(N)nccc45)cc3c2COc2ccccc2CC(=O)O)C1]

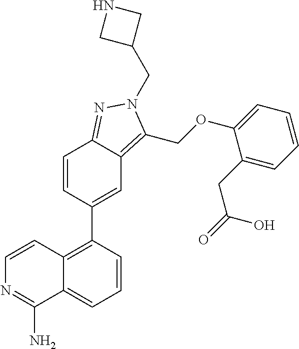 [SMILES: Nc1nccc2c(-c3ccc4nn(CC5CNC5)c(COc5ccccc5CC(=O)O)c4c3)cccc12]

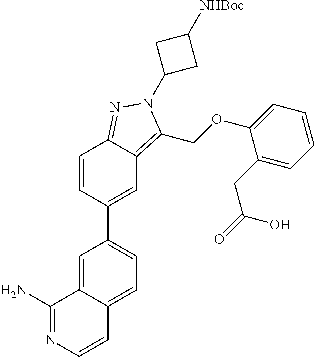 [SMILES: CC(C)(C)OC(=O)NC1CC(n2nc3ccc(-c4ccc5ccnc(N)c5c4)cc3c2COc2ccccc2CC(=O)O)C1]

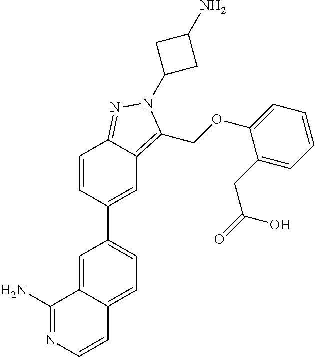 [SMILES: Nc1nccc2ccc(-c3ccc4nn(C5CC(N)C5)c(COc5ccccc5CC(=O)O)c4c3)cc12]

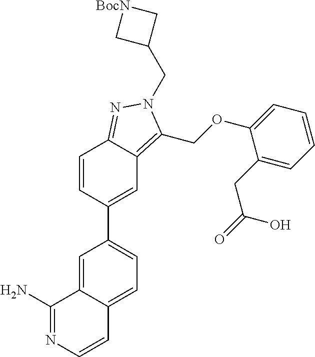 [SMILES: CC(C)(C)OC(=O)N1CC(Cn2nc3ccc(-c4ccc5ccnc(N)c5c4)cc3c2COc2ccccc2CC(=O)O)C1]

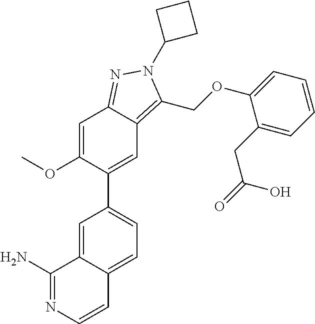 [SMILES: COc1cc2nn(C3CCC3)c(COc3ccccc3CC(=O)O)c2cc1-c1ccc2ccnc(N)c2c1]

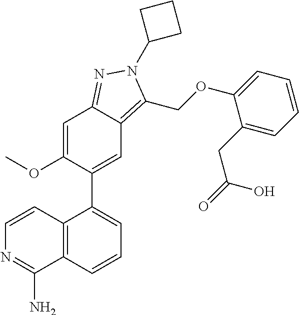 [SMILES: COc1cc2nn(C3CCC3)c(COc3ccccc3CC(=O)O)c2cc1-c1cccc2c(N)nccc12]

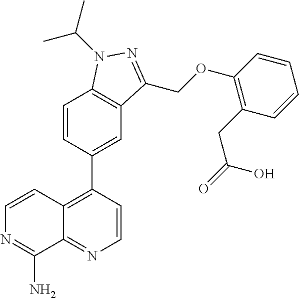 [SMILES: CC(C)n1nc(COc2ccccc2CC(=O)O)c2cc(-c3ccnc4c(N)nccc34)ccc21]

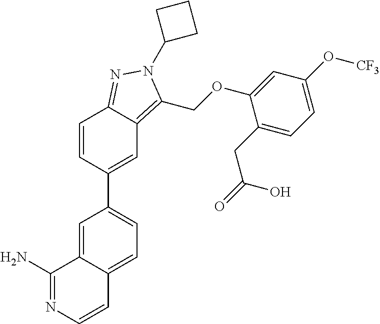 [SMILES: Nc1nccc2ccc(-c3ccc4nn(C5CCC5)c(COc5cc(OC(F)(F)F)ccc5CC(=O)O)c4c3)cc12]

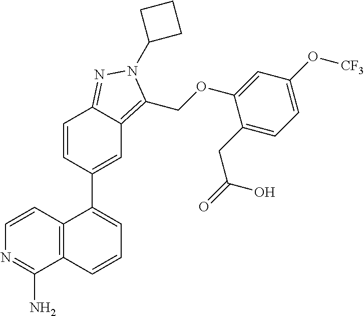 [SMILES: Nc1nccc2c(-c3ccc4nn(C5CCC5)c(COc5cc(OC(F)(F)F)ccc5CC(=O)O)c4c3)cccc12]

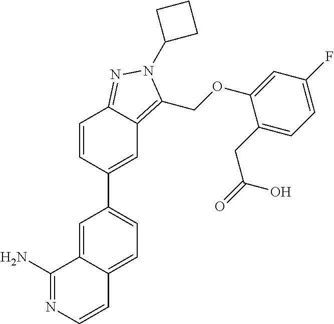 [SMILES: Nc1nccc2ccc(-c3ccc4nn(C5CCC5)c(COc5cc(F)ccc5CC(=O)O)c4c3)cc12]

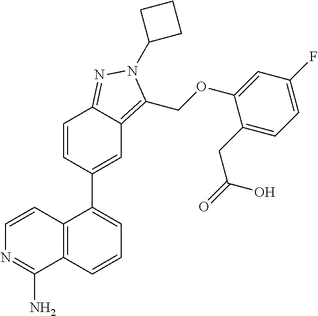 [SMILES: Nc1nccc2c(-c3ccc4nn(C5CCC5)c(COc5cc(F)ccc5CC(=O)O)c4c3)cccc12]

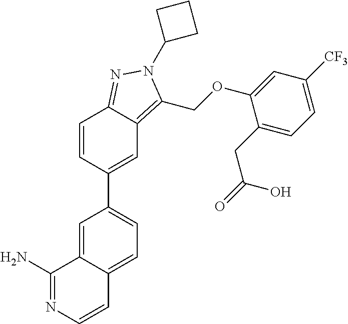 [SMILES: Nc1nccc2ccc(-c3ccc4nn(C5CCC5)c(COc5cc(C(F)(F)F)ccc5CC(=O)O)c4c3)cc12]

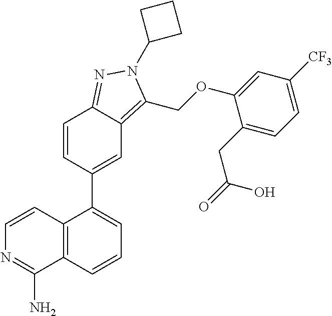 [SMILES: Nc1nccc2c(-c3ccc4nn(C5CCC5)c(COc5cc(C(F)(F)F)ccc5CC(=O)O)c4c3)cccc12]

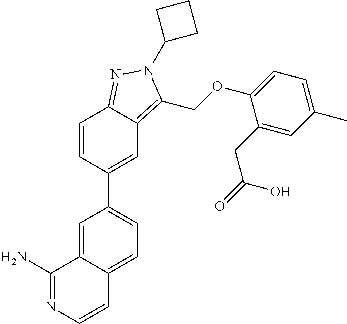 [SMILES: Cc1ccc(OCc2c3cc(-c4ccc5ccnc(N)c5c4)ccc3nn2C2CCC2)c(CC(=O)O)c1]

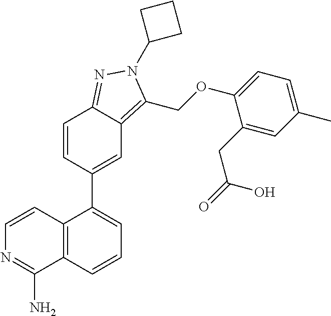 [SMILES: Cc1ccc(OCc2c3cc(-c4cccc5c(N)nccc45)ccc3nn2C2CCC2)c(CC(=O)O)c1]

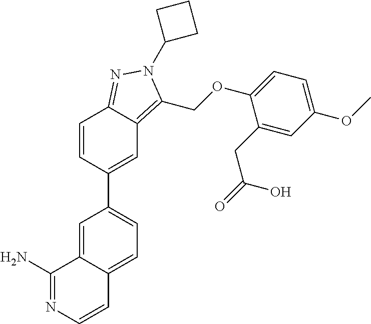 [SMILES: COc1ccc(OCc2c3cc(-c4ccc5ccnc(N)c5c4)ccc3nn2C2CCC2)c(CC(=O)O)c1]

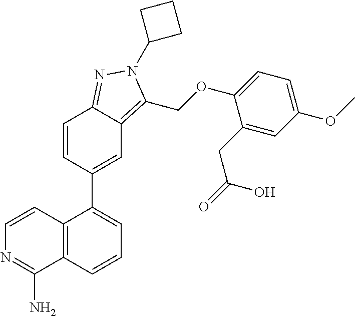 [SMILES: COc1ccc(OCc2c3cc(-c4cccc5c(N)nccc45)ccc3nn2C2CCC2)c(CC(=O)O)c1]

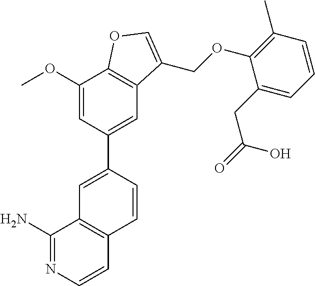 [SMILES: COc1cc(-c2ccc3ccnc(N)c3c2)cc2c(COc3c(C)cccc3CC(=O)O)coc12]

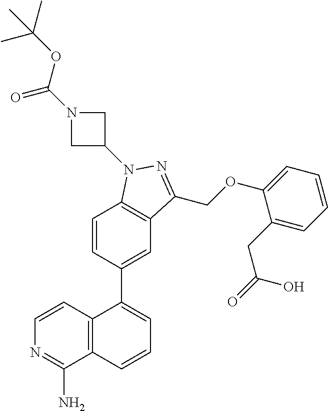 [SMILES: CC(C)(C)OC(=O)N1CC(n2nc(COc3ccccc3CC(=O)O)c3cc(-c4cccc5c(N)nccc45)ccc32)C1]

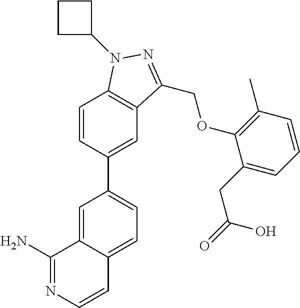 [SMILES: Cc1cccc(CC(=O)O)c1OCc1nn(C2CCC2)c2ccc(-c3ccc4ccnc(N)c4c3)cc12]

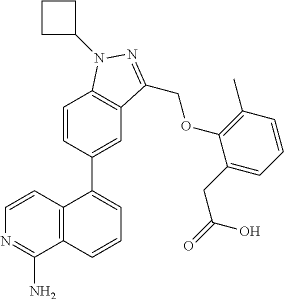 [SMILES: Cc1cccc(CC(=O)O)c1OCc1nn(C2CCC2)c2ccc(-c3cccc4c(N)nccc34)cc12]

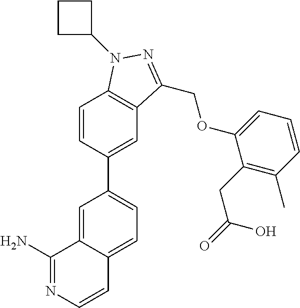 [SMILES: Cc1cccc(OCc2nn(C3CCC3)c3ccc(-c4ccc5ccnc(N)c5c4)cc23)c1CC(=O)O]

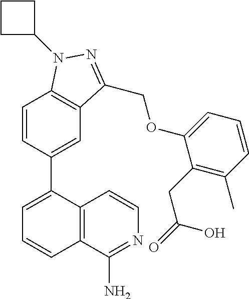 [SMILES: Cc1cccc(OCc2nn(C3CCC3)c3ccc(-c4cccc5c(N)nccc45)cc23)c1CC(=O)O]

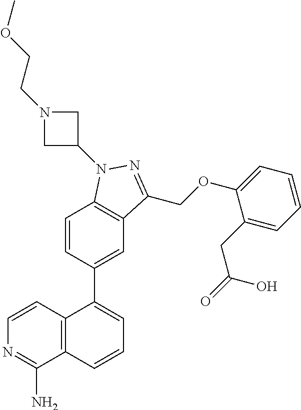 [SMILES: COCCN1CC(n2nc(COc3ccccc3CC(=O)O)c3cc(-c4cccc5c(N)nccc45)ccc32)C1]

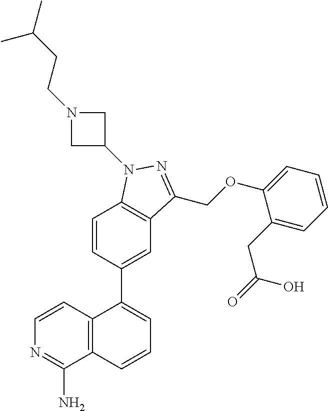 [SMILES: CC(C)CCN1CC(n2nc(COc3ccccc3CC(=O)O)c3cc(-c4cccc5c(N)nccc45)ccc32)C1]

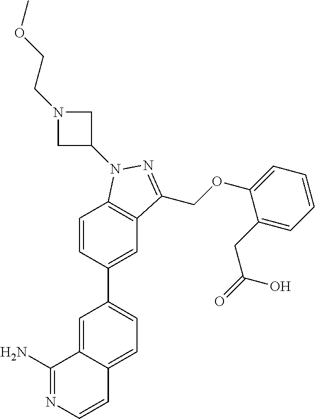 [SMILES: COCCN1CC(n2nc(COc3ccccc3CC(=O)O)c3cc(-c4ccc5ccnc(N)c5c4)ccc32)C1]

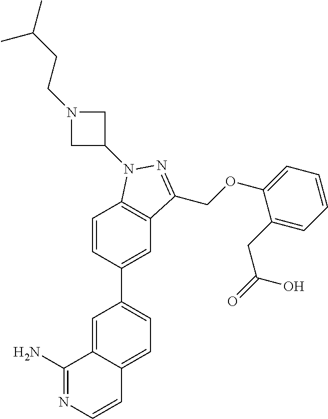 [SMILES: CC(C)CCN1CC(n2nc(COc3ccccc3CC(=O)O)c3cc(-c4ccc5ccnc(N)c5c4)ccc32)C1]

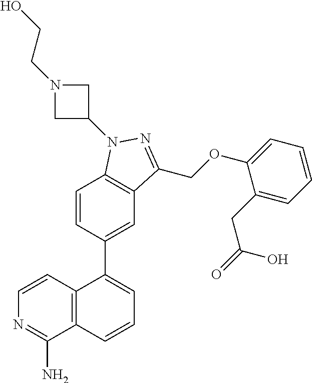 [SMILES: Nc1nccc2c(-c3ccc4c(c3)c(COc3ccccc3CC(=O)O)nn4C3CN(CCO)C3)cccc12]

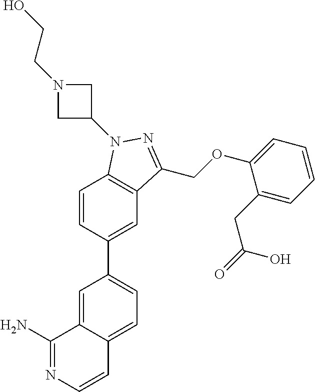 [SMILES: Nc1nccc2ccc(-c3ccc4c(c3)c(COc3ccccc3CC(=O)O)nn4C3CN(CCO)C3)cc12]

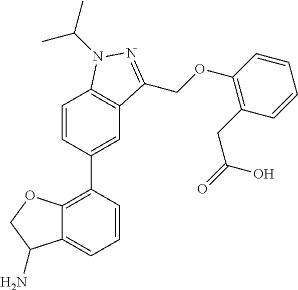 [SMILES: CC(C)n1nc(COc2ccccc2CC(=O)O)c2cc(-c3cccc4c3OCC4N)ccc21]

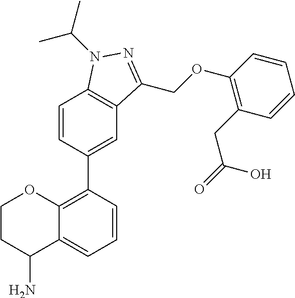 [SMILES: CC(C)n1nc(COc2ccccc2CC(=O)O)c2cc(-c3cccc4c3OCCC4N)ccc21]

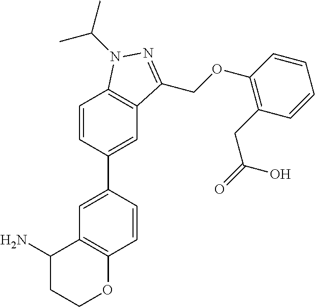 [SMILES: CC(C)n1nc(COc2ccccc2CC(=O)O)c2cc(-c3ccc4c(c3)C(N)CCO4)ccc21]

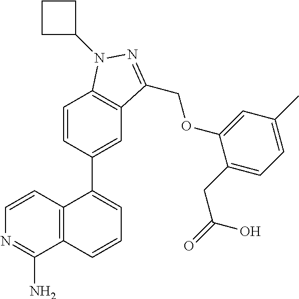 [SMILES: Cc1ccc(CC(=O)O)c(OCc2nn(C3CCC3)c3ccc(-c4cccc5c(N)nccc45)cc23)c1]

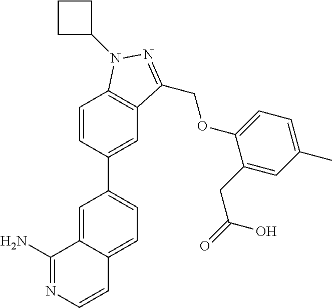 [SMILES: Cc1ccc(OCc2nn(C3CCC3)c3ccc(-c4ccc5ccnc(N)c5c4)cc23)c(CC(=O)O)c1]

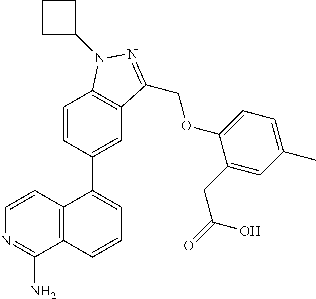 [SMILES: Cc1ccc(OCc2nn(C3CCC3)c3ccc(-c4cccc5c(N)nccc45)cc23)c(CC(=O)O)c1]

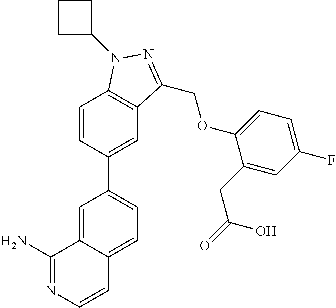 [SMILES: Nc1nccc2ccc(-c3ccc4c(c3)c(COc3ccc(F)cc3CC(=O)O)nn4C3CCC3)cc12]

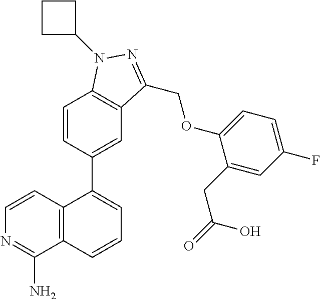 [SMILES: Nc1nccc2c(-c3ccc4c(c3)c(COc3ccc(F)cc3CC(=O)O)nn4C3CCC3)cccc12]

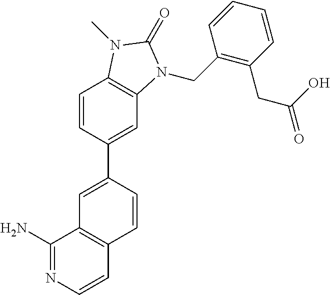 [SMILES: Cn1c(=O)n(Cc2ccccc2CC(=O)O)c2cc(-c3ccc4ccnc(N)c4c3)ccc21]

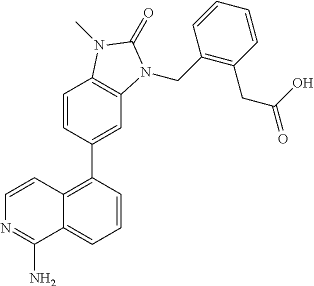 [SMILES: Cn1c(=O)n(Cc2ccccc2CC(=O)O)c2cc(-c3cccc4c(N)nccc34)ccc21]

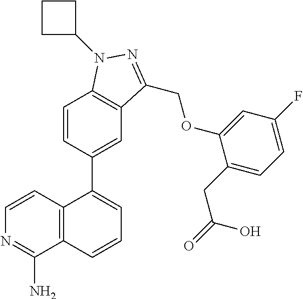 [SMILES: Nc1nccc2c(-c3ccc4c(c3)c(COc3cc(F)ccc3CC(=O)O)nn4C3CCC3)cccc12]

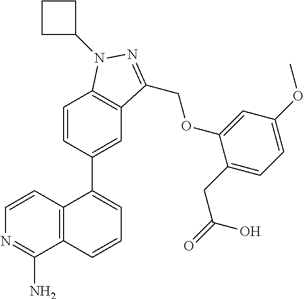 [SMILES: COc1ccc(CC(=O)O)c(OCc2nn(C3CCC3)c3ccc(-c4cccc5c(N)nccc45)cc23)c1]

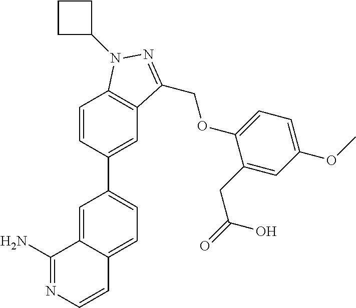 [SMILES: COc1ccc(OCc2nn(C3CCC3)c3ccc(-c4ccc5ccnc(N)c5c4)cc23)c(CC(=O)O)c1]

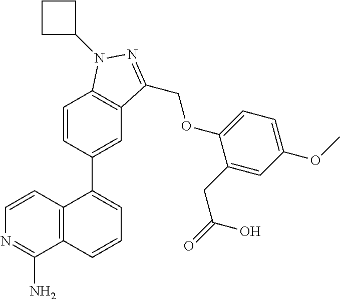 [SMILES: COc1ccc(OCc2nn(C3CCC3)c3ccc(-c4cccc5c(N)nccc45)cc23)c(CC(=O)O)c1]

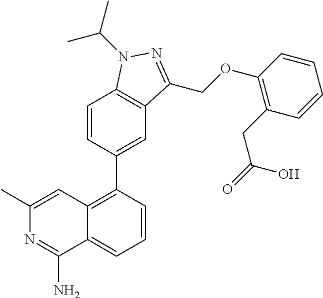 [SMILES: Cc1cc2c(-c3ccc4c(c3)c(COc3ccccc3CC(=O)O)nn4C(C)C)cccc2c(N)n1]

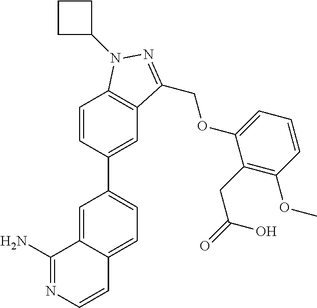 [SMILES: COc1cccc(OCc2nn(C3CCC3)c3ccc(-c4ccc5ccnc(N)c5c4)cc23)c1CC(=O)O]

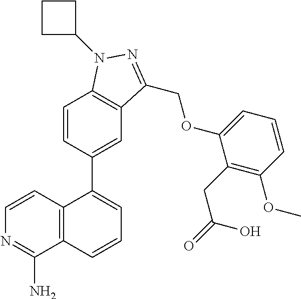 [SMILES: COc1cccc(OCc2nn(C3CCC3)c3ccc(-c4cccc5c(N)nccc45)cc23)c1CC(=O)O]

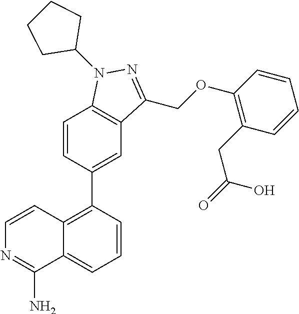 [SMILES: Nc1nccc2c(-c3ccc4c(c3)c(COc3ccccc3CC(=O)O)nn4C3CCCC3)cccc12]

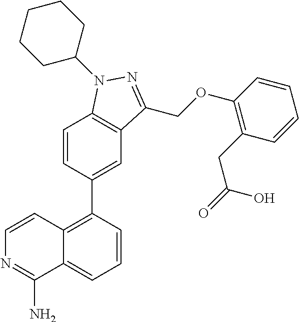 [SMILES: Nc1nccc2c(-c3ccc4c(c3)c(COc3ccccc3CC(=O)O)nn4C3CCCCC3)cccc12]

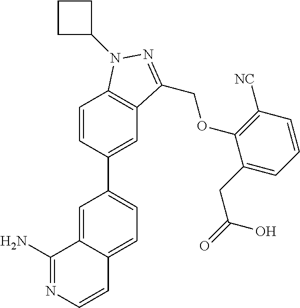 [SMILES: N#Cc1cccc(CC(=O)O)c1OCc1nn(C2CCC2)c2ccc(-c3ccc4ccnc(N)c4c3)cc12]

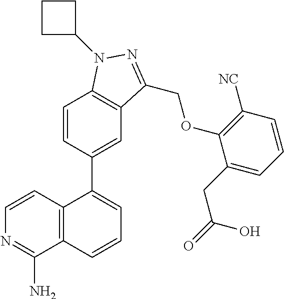 [SMILES: N#Cc1cccc(CC(=O)O)c1OCc1nn(C2CCC2)c2ccc(-c3cccc4c(N)nccc34)cc12]

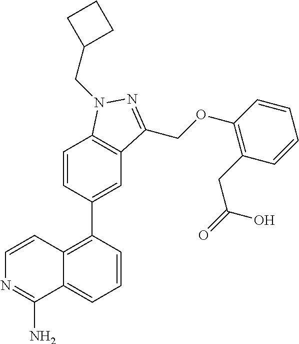 [SMILES: Nc1nccc2c(-c3ccc4c(c3)c(COc3ccccc3CC(=O)O)nn4CC3CCC3)cccc12]

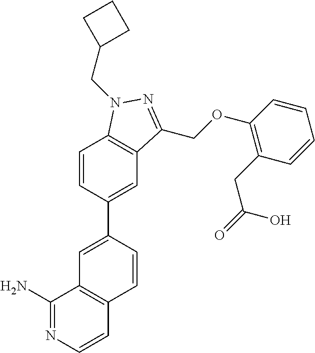 [SMILES: Nc1nccc2ccc(-c3ccc4c(c3)c(COc3ccccc3CC(=O)O)nn4CC3CCC3)cc12]

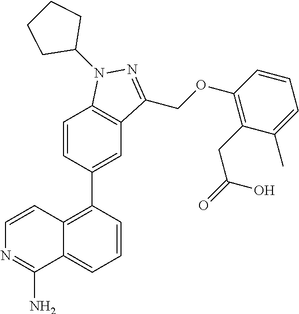 [SMILES: Cc1cccc(OCc2nn(C3CCCC3)c3ccc(-c4cccc5c(N)nccc45)cc23)c1CC(=O)O]

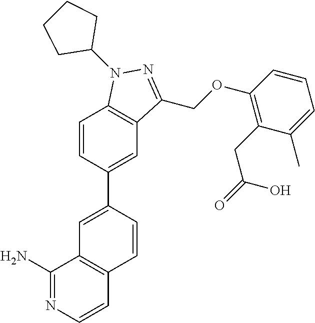 [SMILES: Cc1cccc(OCc2nn(C3CCCC3)c3ccc(-c4ccc5ccnc(N)c5c4)cc23)c1CC(=O)O]

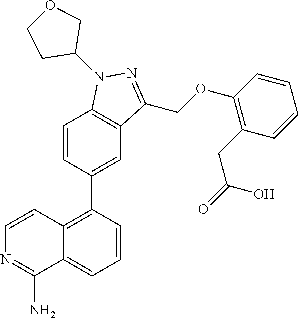 [SMILES: Nc1nccc2c(-c3ccc4c(c3)c(COc3ccccc3CC(=O)O)nn4C3CCOC3)cccc12]

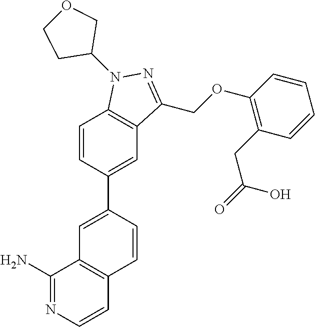 [SMILES: Nc1nccc2ccc(-c3ccc4c(c3)c(COc3ccccc3CC(=O)O)nn4C3CCOC3)cc12]

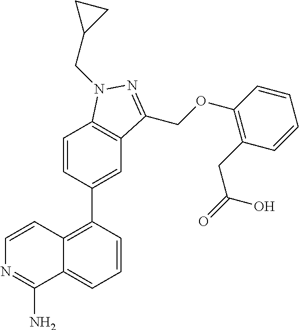 [SMILES: Nc1nccc2c(-c3ccc4c(c3)c(COc3ccccc3CC(=O)O)nn4CC3CC3)cccc12]

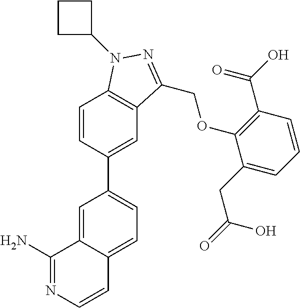 [SMILES: Nc1nccc2ccc(-c3ccc4c(c3)c(COc3c(CC(=O)O)cccc3C(=O)O)nn4C3CCC3)cc12]

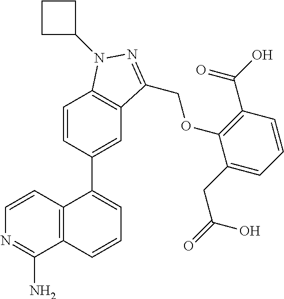 [SMILES: Nc1nccc2c(-c3ccc4c(c3)c(COc3c(CC(=O)O)cccc3C(=O)O)nn4C3CCC3)cccc12]

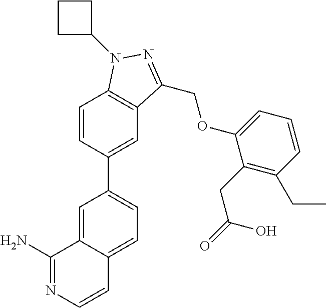 [SMILES: CCc1cccc(OCc2nn(C3CCC3)c3ccc(-c4ccc5ccnc(N)c5c4)cc23)c1CC(=O)O]

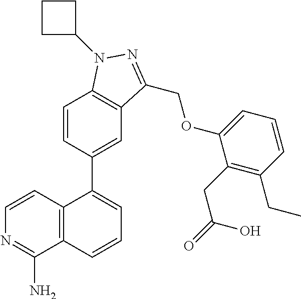 [SMILES: CCc1cccc(OCc2nn(C3CCC3)c3ccc(-c4cccc5c(N)nccc45)cc23)c1CC(=O)O]

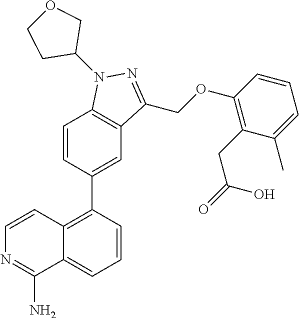 [SMILES: Cc1cccc(OCc2nn(C3CCOC3)c3ccc(-c4cccc5c(N)nccc45)cc23)c1CC(=O)O]

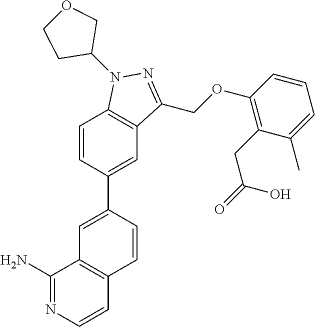 [SMILES: Cc1cccc(OCc2nn(C3CCOC3)c3ccc(-c4ccc5ccnc(N)c5c4)cc23)c1CC(=O)O]

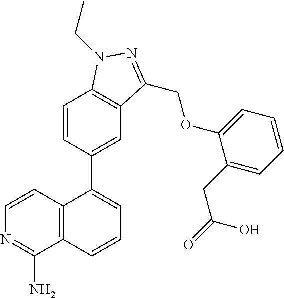 [SMILES: CCn1nc(COc2ccccc2CC(=O)O)c2cc(-c3cccc4c(N)nccc34)ccc21]

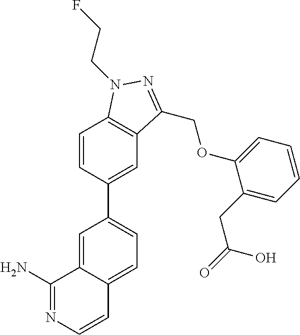 [SMILES: Nc1nccc2ccc(-c3ccc4c(c3)c(COc3ccccc3CC(=O)O)nn4CCF)cc12]

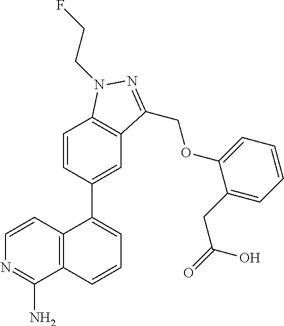 [SMILES: Nc1nccc2c(-c3ccc4c(c3)c(COc3ccccc3CC(=O)O)nn4CCF)cccc12]

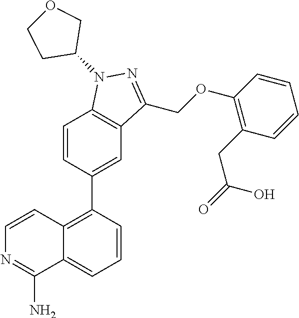 [SMILES: Nc1nccc2c(-c3ccc4c(c3)c(COc3ccccc3CC(=O)O)nn4[C@@H]3CCOC3)cccc12]

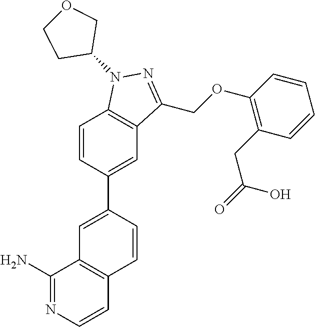 [SMILES: Nc1nccc2ccc(-c3ccc4c(c3)c(COc3ccccc3CC(=O)O)nn4[C@@H]3CCOC3)cc12]

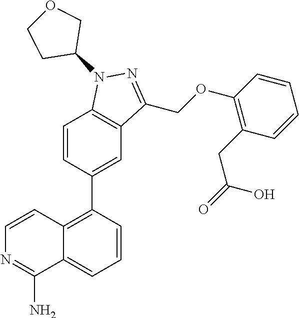 [SMILES: Nc1nccc2c(-c3ccc4c(c3)c(COc3ccccc3CC(=O)O)nn4[C@H]3CCOC3)cccc12]

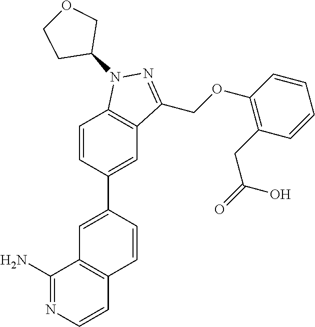 [SMILES: Nc1nccc2ccc(-c3ccc4c(c3)c(COc3ccccc3CC(=O)O)nn4[C@H]3CCOC3)cc12]

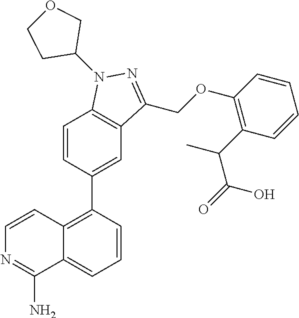 [SMILES: CC(C(=O)O)c1ccccc1OCc1nn(C2CCOC2)c2ccc(-c3cccc4c(N)nccc34)cc12]

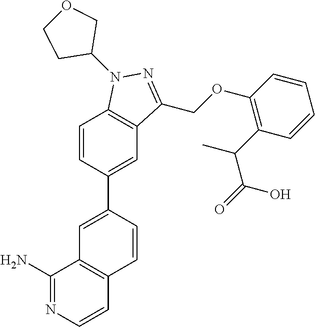 [SMILES: CC(C(=O)O)c1ccccc1OCc1nn(C2CCOC2)c2ccc(-c3ccc4ccnc(N)c4c3)cc12]

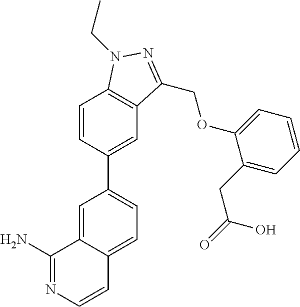 [SMILES: CCn1nc(COc2ccccc2CC(=O)O)c2cc(-c3ccc4ccnc(N)c4c3)ccc21]

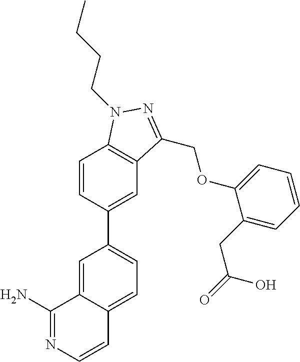 [SMILES: CCCCn1nc(COc2ccccc2CC(=O)O)c2cc(-c3ccc4ccnc(N)c4c3)ccc21]

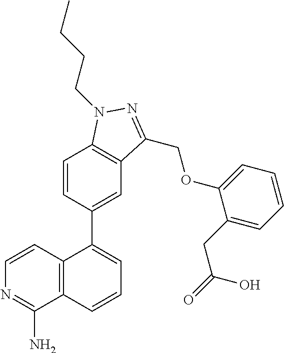 [SMILES: CCCCn1nc(COc2ccccc2CC(=O)O)c2cc(-c3cccc4c(N)nccc34)ccc21]

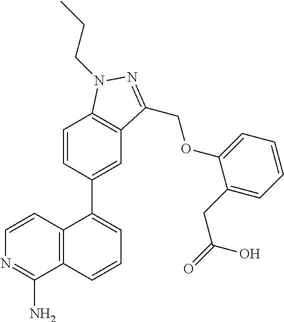 [SMILES: CCCn1nc(COc2ccccc2CC(=O)O)c2cc(-c3cccc4c(N)nccc34)ccc21]

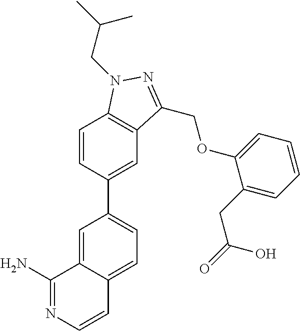 [SMILES: CC(C)Cn1nc(COc2ccccc2CC(=O)O)c2cc(-c3ccc4ccnc(N)c4c3)ccc21]

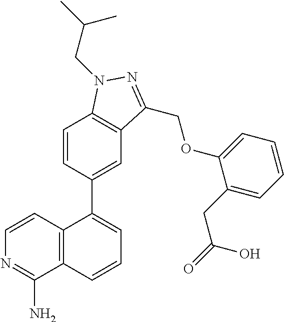 [SMILES: CC(C)Cn1nc(COc2ccccc2CC(=O)O)c2cc(-c3cccc4c(N)nccc34)ccc21]

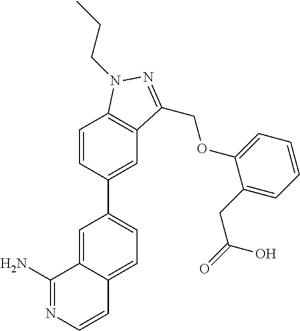 [SMILES: CCCn1nc(COc2ccccc2CC(=O)O)c2cc(-c3ccc4ccnc(N)c4c3)ccc21]

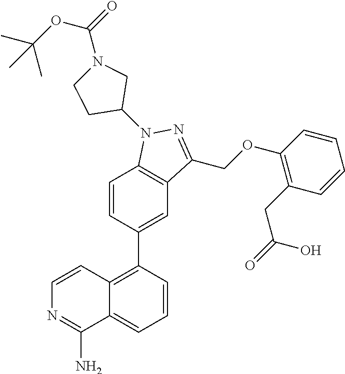 [SMILES: CC(C)(C)OC(=O)N1CCC(n2nc(COc3ccccc3CC(=O)O)c3cc(-c4cccc5c(N)nccc45)ccc32)C1]

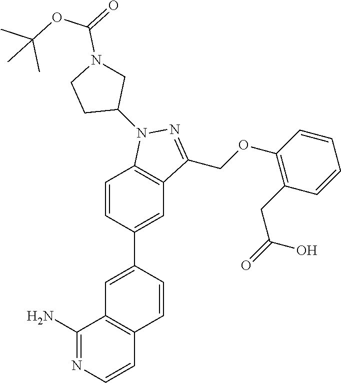 [SMILES: CC(C)(C)OC(=O)N1CCC(n2nc(COc3ccccc3CC(=O)O)c3cc(-c4ccc5ccnc(N)c5c4)ccc32)C1]

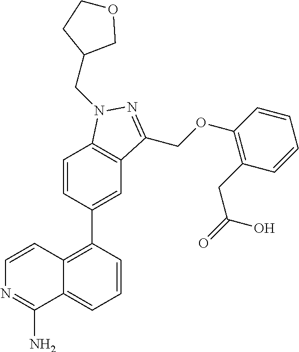 [SMILES: Nc1nccc2c(-c3ccc4c(c3)c(COc3ccccc3CC(=O)O)nn4CC3CCOC3)cccc12]

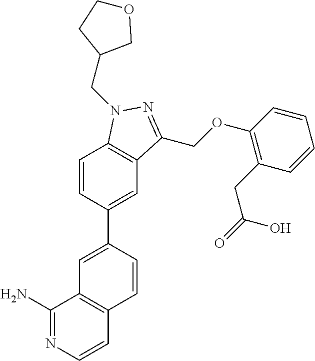 [SMILES: Nc1nccc2ccc(-c3ccc4c(c3)c(COc3ccccc3CC(=O)O)nn4CC3CCOC3)cc12]

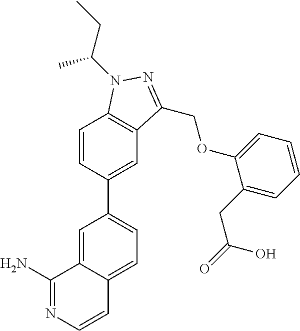 [SMILES: CC[C@@H](C)n1nc(COc2ccccc2CC(=O)O)c2cc(-c3ccc4ccnc(N)c4c3)ccc21]

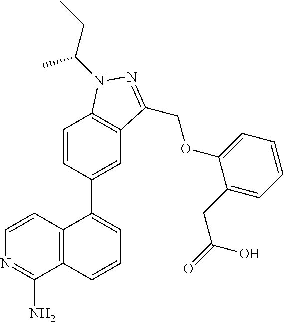 [SMILES: CC[C@@H](C)n1nc(COc2ccccc2CC(=O)O)c2cc(-c3cccc4c(N)nccc34)ccc21]

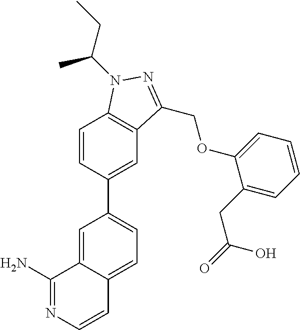 [SMILES: CC[C@H](C)n1nc(COc2ccccc2CC(=O)O)c2cc(-c3ccc4ccnc(N)c4c3)ccc21]

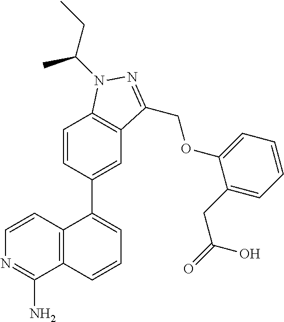 [SMILES: CC[C@H](C)n1nc(COc2ccccc2CC(=O)O)c2cc(-c3cccc4c(N)nccc34)ccc21]

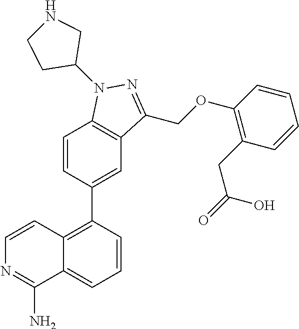 [SMILES: Nc1nccc2c(-c3ccc4c(c3)c(COc3ccccc3CC(=O)O)nn4C3CCNC3)cccc12]

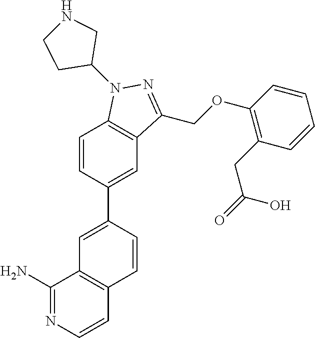 [SMILES: Nc1nccc2ccc(-c3ccc4c(c3)c(COc3ccccc3CC(=O)O)nn4C3CCNC3)cc12]

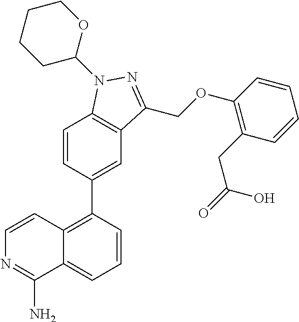 [SMILES: Nc1nccc2c(-c3ccc4c(c3)c(COc3ccccc3CC(=O)O)nn4C3CCCCO3)cccc12]

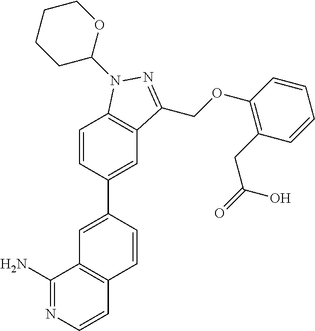 [SMILES: Nc1nccc2ccc(-c3ccc4c(c3)c(COc3ccccc3CC(=O)O)nn4C3CCCCO3)cc12]

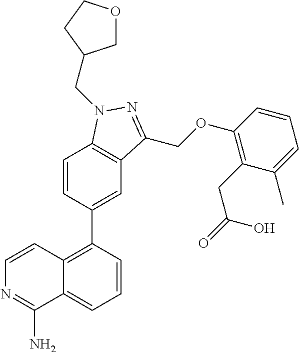 [SMILES: Cc1cccc(OCc2nn(CC3CCOC3)c3ccc(-c4cccc5c(N)nccc45)cc23)c1CC(=O)O]

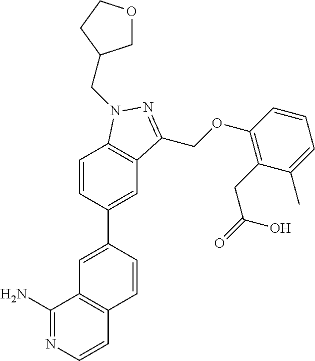 [SMILES: Cc1cccc(OCc2nn(CC3CCOC3)c3ccc(-c4ccc5ccnc(N)c5c4)cc23)c1CC(=O)O]

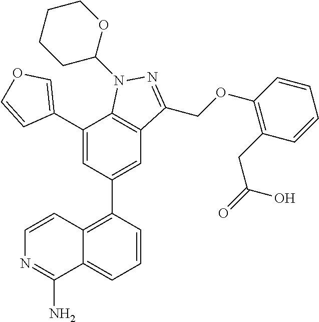 [SMILES: Nc1nccc2c(-c3cc(-c4ccoc4)c4c(c3)c(COc3ccccc3CC(=O)O)nn4C3CCCCO3)cccc12]

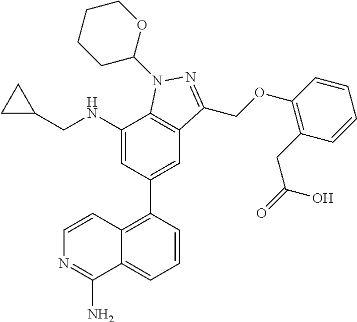 [SMILES: Nc1nccc2c(-c3cc(NCC4CC4)c4c(c3)c(COc3ccccc3CC(=O)O)nn4C3CCCCO3)cccc12]

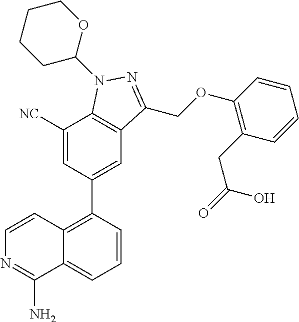 [SMILES: N#Cc1cc(-c2cccc3c(N)nccc23)cc2c(COc3ccccc3CC(=O)O)nn(C3CCCCO3)c12]

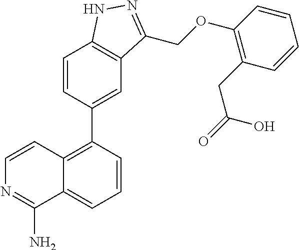 [SMILES: Nc1nccc2c(-c3ccc4[nH]nc(COc5ccccc5CC(=O)O)c4c3)cccc12]

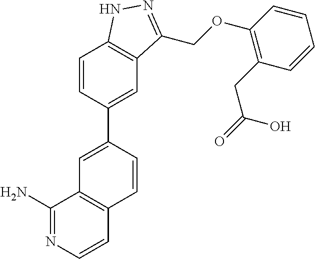 [SMILES: Nc1nccc2ccc(-c3ccc4[nH]nc(COc5ccccc5CC(=O)O)c4c3)cc12]

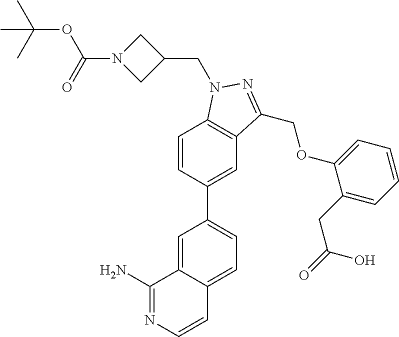 [SMILES: CC(C)(C)OC(=O)N1CC(Cn2nc(COc3ccccc3CC(=O)O)c3cc(-c4ccc5ccnc(N)c5c4)ccc32)C1]

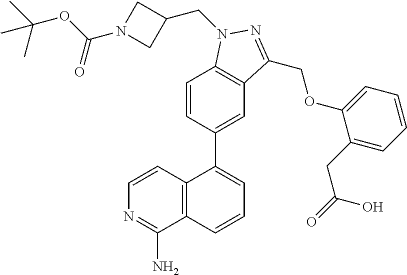 [SMILES: CC(C)(C)OC(=O)N1CC(Cn2nc(COc3ccccc3CC(=O)O)c3cc(-c4cccc5c(N)nccc45)ccc32)C1]

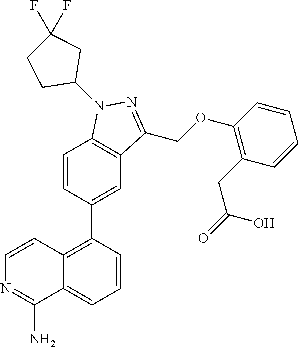 [SMILES: Nc1nccc2c(-c3ccc4c(c3)c(COc3ccccc3CC(=O)O)nn4C3CCC(F)(F)C3)cccc12]

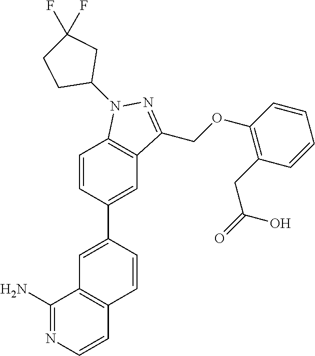 [SMILES: Nc1nccc2ccc(-c3ccc4c(c3)c(COc3ccccc3CC(=O)O)nn4C3CCC(F)(F)C3)cc12]

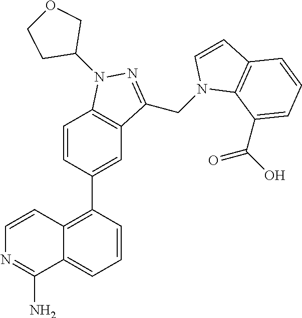 [SMILES: Nc1nccc2c(-c3ccc4c(c3)c(Cn3ccc5cccc(C(=O)O)c53)nn4C3CCOC3)cccc12]

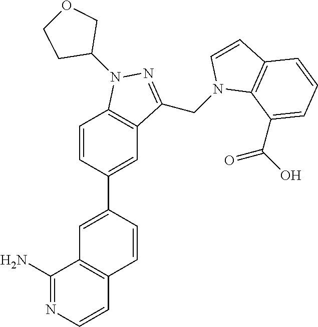 [SMILES: Nc1nccc2ccc(-c3ccc4c(c3)c(Cn3ccc5cccc(C(=O)O)c53)nn4C3CCOC3)cc12]

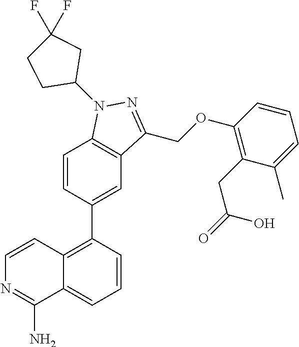 [SMILES: Cc1cccc(OCc2nn(C3CCC(F)(F)C3)c3ccc(-c4cccc5c(N)nccc45)cc23)c1CC(=O)O]

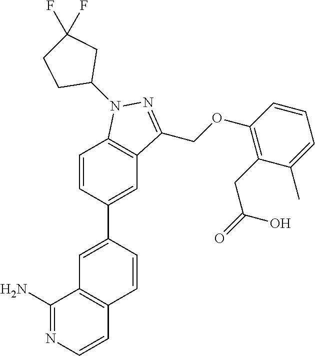 [SMILES: Cc1cccc(OCc2nn(C3CCC(F)(F)C3)c3ccc(-c4ccc5ccnc(N)c5c4)cc23)c1CC(=O)O]

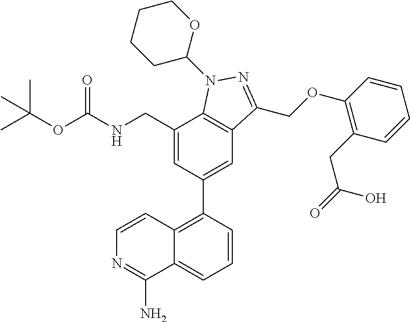 [SMILES: CC(C)(C)OC(=O)NCc1cc(-c2cccc3c(N)nccc23)cc2c(COc3ccccc3CC(=O)O)nn(C3CCCCO3)c12]

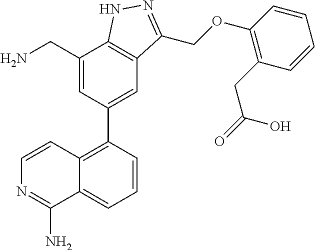 [SMILES: NCc1cc(-c2cccc3c(N)nccc23)cc2c(COc3ccccc3CC(=O)O)n[nH]c12]

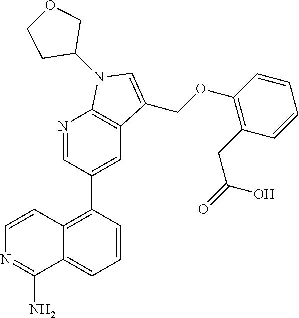 [SMILES: Nc1nccc2c(-c3cnc4c(c3)c(COc3ccccc3CC(=O)O)cn4C3CCOC3)cccc12]

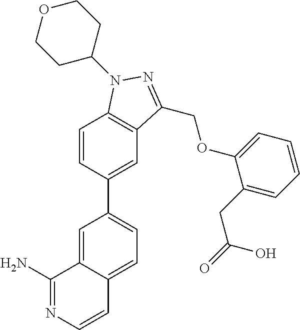 [SMILES: Nc1nccc2ccc(-c3ccc4c(c3)c(COc3ccccc3CC(=O)O)nn4C3CCOCC3)cc12]

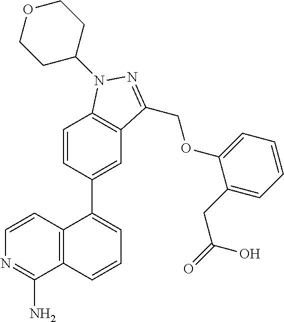 [SMILES: Nc1nccc2c(-c3ccc4c(c3)c(COc3ccccc3CC(=O)O)nn4C3CCOCC3)cccc12]

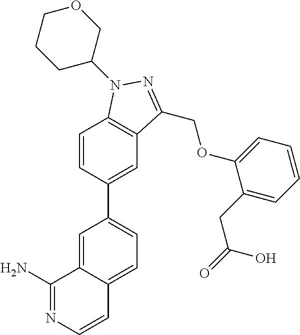 [SMILES: Nc1nccc2ccc(-c3ccc4c(c3)c(COc3ccccc3CC(=O)O)nn4C3CCCOC3)cc12]

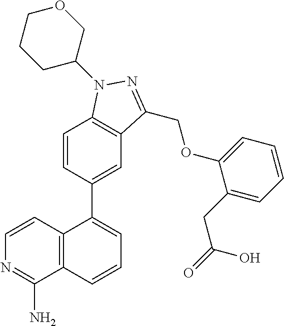 [SMILES: Nc1nccc2c(-c3ccc4c(c3)c(COc3ccccc3CC(=O)O)nn4C3CCCOC3)cccc12]

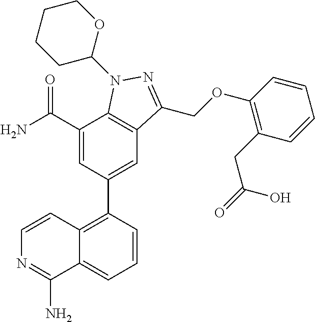 [SMILES: NC(=O)c1cc(-c2cccc3c(N)nccc23)cc2c(COc3ccccc3CC(=O)O)nn(C3CCCCO3)c12]

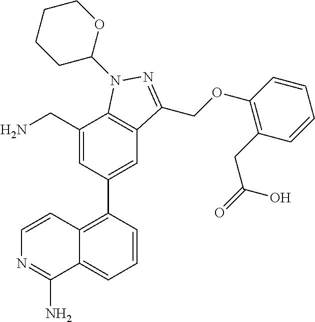 [SMILES: NCc1cc(-c2cccc3c(N)nccc23)cc2c(COc3ccccc3CC(=O)O)nn(C3CCCCO3)c12]

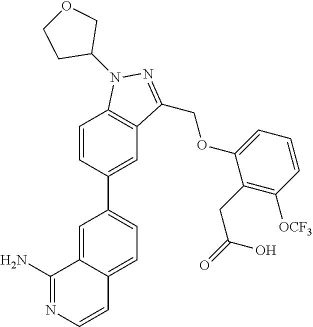 [SMILES: Nc1nccc2ccc(-c3ccc4c(c3)c(COc3cccc(OC(F)(F)F)c3CC(=O)O)nn4C3CCOC3)cc12]

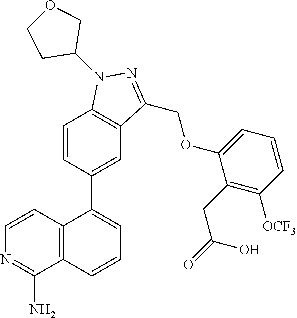 [SMILES: Nc1nccc2c(-c3ccc4c(c3)c(COc3cccc(OC(F)(F)F)c3CC(=O)O)nn4C3CCOC3)cccc12]

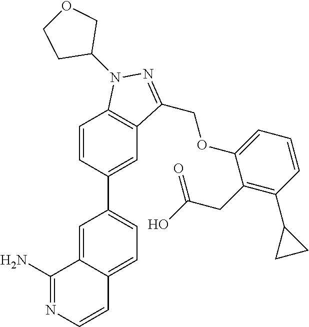 [SMILES: Nc1nccc2ccc(-c3ccc4c(c3)c(COc3cccc(C5CC5)c3CC(=O)O)nn4C3CCOC3)cc12]

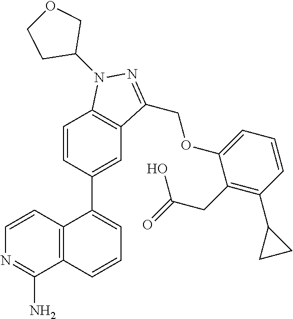 [SMILES: Nc1nccc2c(-c3ccc4c(c3)c(COc3cccc(C5CC5)c3CC(=O)O)nn4C3CCOC3)cccc12]

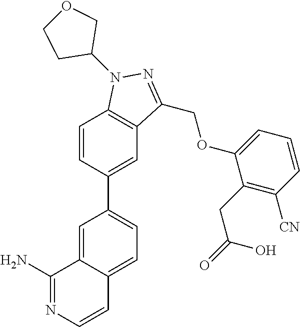 [SMILES: N#Cc1cccc(OCc2nn(C3CCOC3)c3ccc(-c4ccc5ccnc(N)c5c4)cc23)c1CC(=O)O]

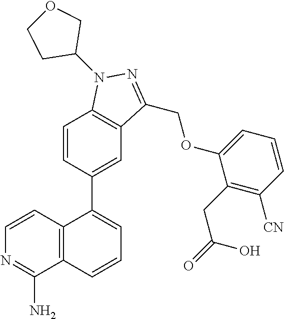 [SMILES: N#Cc1cccc(OCc2nn(C3CCOC3)c3ccc(-c4cccc5c(N)nccc45)cc23)c1CC(=O)O]

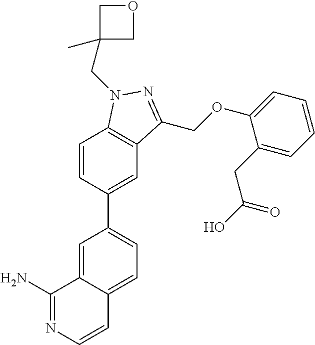 [SMILES: CC1(Cn2nc(COc3ccccc3CC(=O)O)c3cc(-c4ccc5ccnc(N)c5c4)ccc32)COC1]

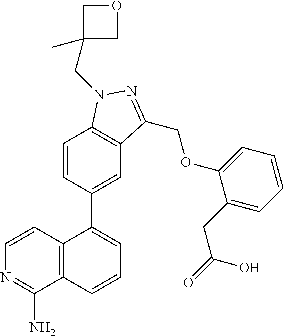 [SMILES: CC1(Cn2nc(COc3ccccc3CC(=O)O)c3cc(-c4cccc5c(N)nccc45)ccc32)COC1]

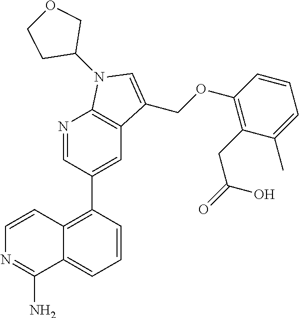 [SMILES: Cc1cccc(OCc2cn(C3CCOC3)c3ncc(-c4cccc5c(N)nccc45)cc23)c1CC(=O)O]

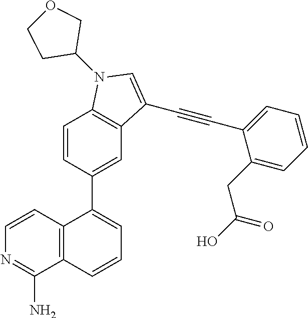 [SMILES: Nc1nccc2c(-c3ccc4c(c3)c(C#Cc3ccccc3CC(=O)O)cn4C3CCOC3)cccc12]

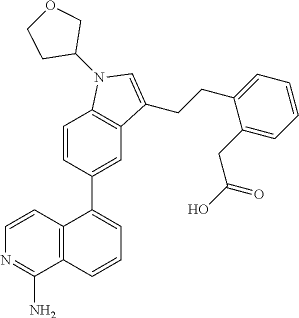 [SMILES: Nc1nccc2c(-c3ccc4c(c3)c(CCc3ccccc3CC(=O)O)cn4C3CCOC3)cccc12]

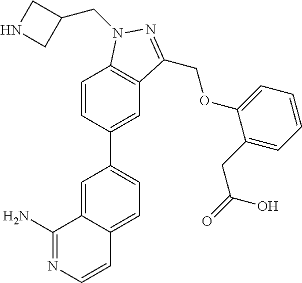 [SMILES: Nc1nccc2ccc(-c3ccc4c(c3)c(COc3ccccc3CC(=O)O)nn4CC3CNC3)cc12]

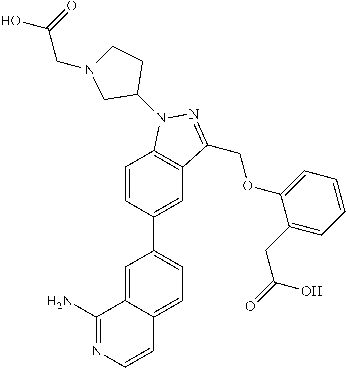 [SMILES: Nc1nccc2ccc(-c3ccc4c(c3)c(COc3ccccc3CC(=O)O)nn4C3CCN(CC(=O)O)C3)cc12]

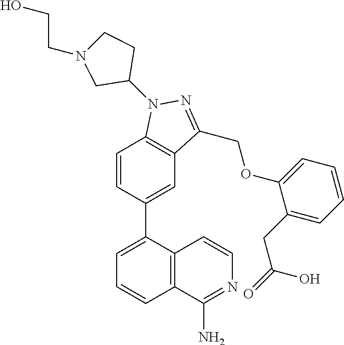 [SMILES: Nc1nccc2c(-c3ccc4c(c3)c(COc3ccccc3CC(=O)O)nn4C3CCN(CCO)C3)cccc12]

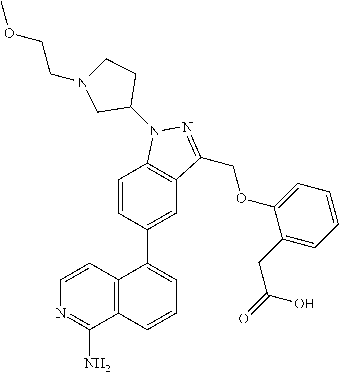 [SMILES: COCCN1CCC(n2nc(COc3ccccc3CC(=O)O)c3cc(-c4cccc5c(N)nccc45)ccc32)C1]

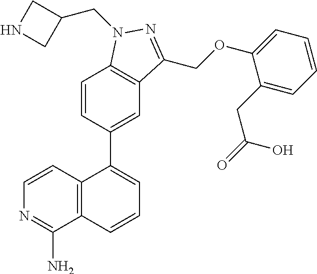 [SMILES: Nc1nccc2c(-c3ccc4c(c3)c(COc3ccccc3CC(=O)O)nn4CC3CNC3)cccc12]

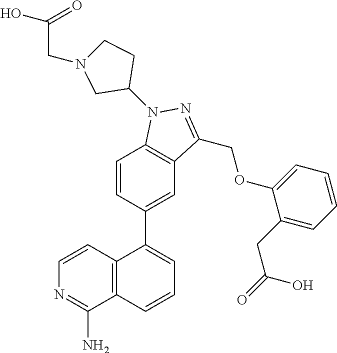 [SMILES: Nc1nccc2c(-c3ccc4c(c3)c(COc3ccccc3CC(=O)O)nn4C3CCN(CC(=O)O)C3)cccc12]

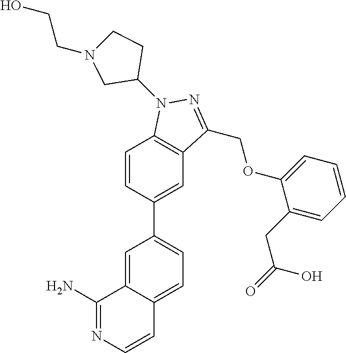 [SMILES: Nc1nccc2ccc(-c3ccc4c(c3)c(COc3ccccc3CC(=O)O)nn4C3CCN(CCO)C3)cc12]

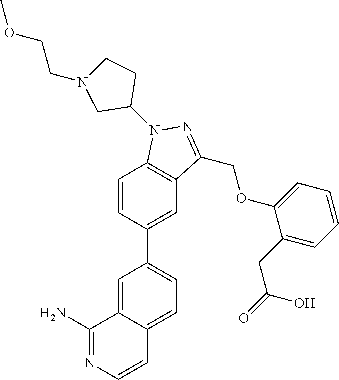 [SMILES: COCCN1CCC(n2nc(COc3ccccc3CC(=O)O)c3cc(-c4ccc5ccnc(N)c5c4)ccc32)C1]

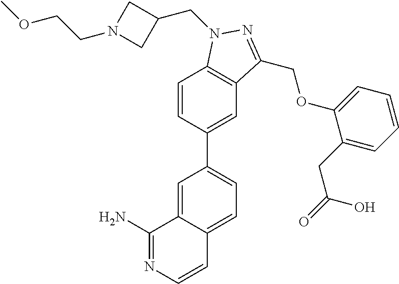 [SMILES: COCCN1CC(Cn2nc(COc3ccccc3CC(=O)O)c3cc(-c4ccc5ccnc(N)c5c4)ccc32)C1]

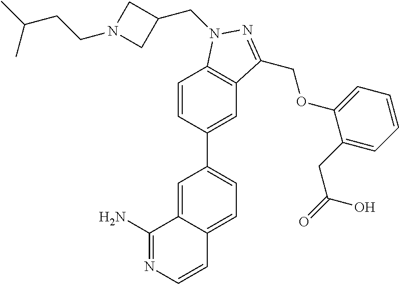 [SMILES: CC(C)CCN1CC(Cn2nc(COc3ccccc3CC(=O)O)c3cc(-c4ccc5ccnc(N)c5c4)ccc32)C1]

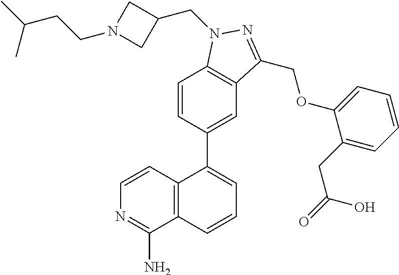 [SMILES: CC(C)CCN1CC(Cn2nc(COc3ccccc3CC(=O)O)c3cc(-c4cccc5c(N)nccc45)ccc32)C1]

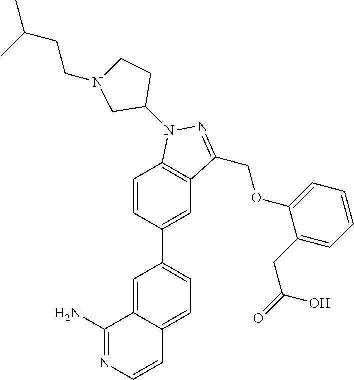 [SMILES: CC(C)CCN1CCC(n2nc(COc3ccccc3CC(=O)O)c3cc(-c4ccc5ccnc(N)c5c4)ccc32)C1]

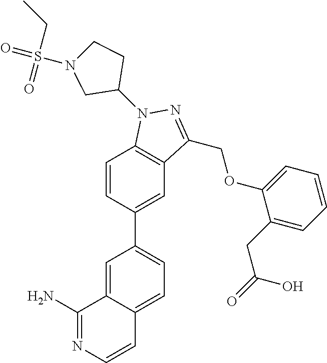 [SMILES: CCS(=O)(=O)N1CCC(n2nc(COc3ccccc3CC(=O)O)c3cc(-c4ccc5ccnc(N)c5c4)ccc32)C1]

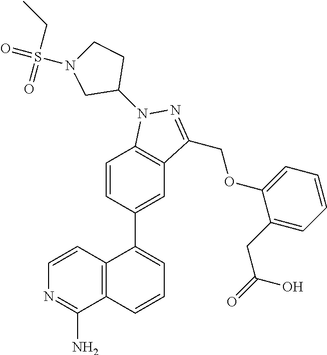 [SMILES: CCS(=O)(=O)N1CCC(n2nc(COc3ccccc3CC(=O)O)c3cc(-c4cccc5c(N)nccc45)ccc32)C1]

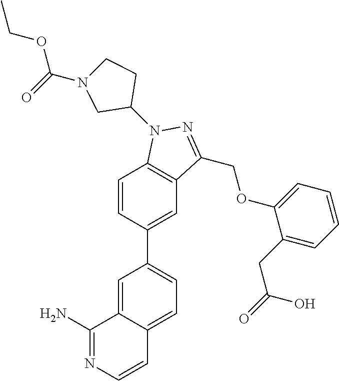 [SMILES: CCOC(=O)N1CCC(n2nc(COc3ccccc3CC(=O)O)c3cc(-c4ccc5ccnc(N)c5c4)ccc32)C1]

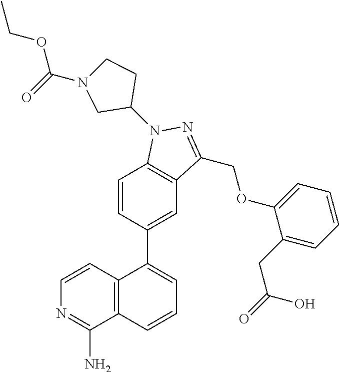 [SMILES: CCOC(=O)N1CCC(n2nc(COc3ccccc3CC(=O)O)c3cc(-c4cccc5c(N)nccc45)ccc32)C1]

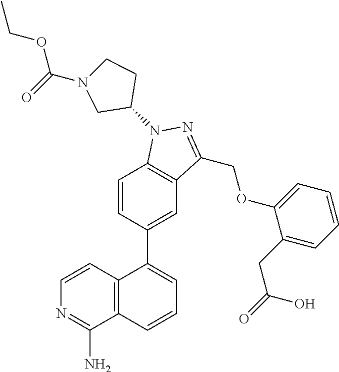 [SMILES: CCOC(=O)N1CC[C@H](n2nc(COc3ccccc3CC(=O)O)c3cc(-c4cccc5c(N)nccc45)ccc32)C1]

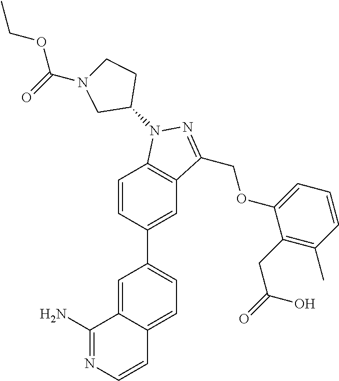 [SMILES: CCOC(=O)N1CC[C@H](n2nc(COc3cccc(C)c3CC(=O)O)c3cc(-c4ccc5ccnc(N)c5c4)ccc32)C1]

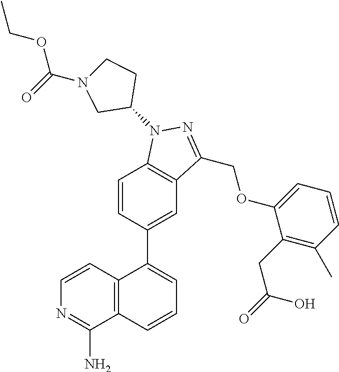 [SMILES: CCOC(=O)N1CC[C@H](n2nc(COc3cccc(C)c3CC(=O)O)c3cc(-c4cccc5c(N)nccc45)ccc32)C1]

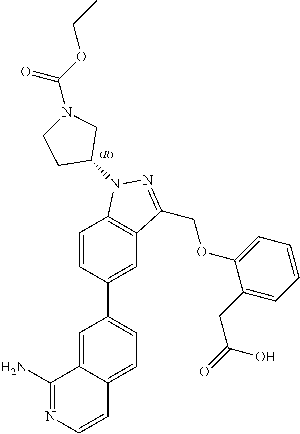

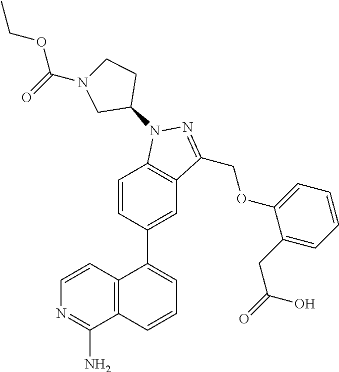 [SMILES: CCOC(=O)N1CC[C@@H](n2nc(COc3ccccc3CC(=O)O)c3cc(-c4cccc5c(N)nccc45)ccc32)C1]

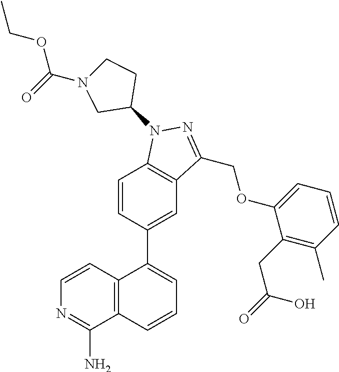 [SMILES: CCOC(=O)N1CC[C@@H](n2nc(COc3cccc(C)c3CC(=O)O)c3cc(-c4cccc5c(N)nccc45)ccc32)C1]

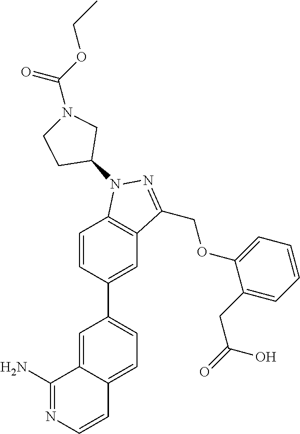 [SMILES: CCOC(=O)N1CC[C@H](n2nc(COc3ccccc3CC(=O)O)c3cc(-c4ccc5ccnc(N)c5c4)ccc32)C1]

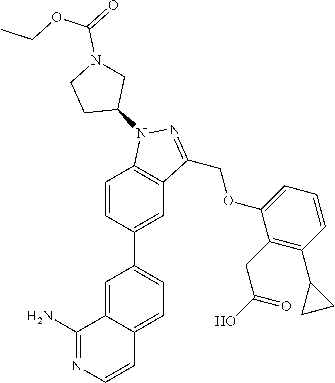 [SMILES: CCOC(=O)N1CC[C@H](n2nc(COc3cccc(C4CC4)c3CC(=O)O)c3cc(-c4ccc5ccnc(N)c5c4)ccc32)C1]

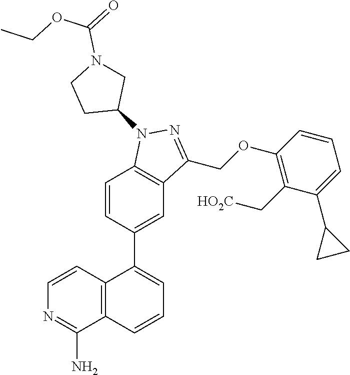 [SMILES: CCOC(=O)N1CC[C@H](n2nc(COc3cccc(C4CC4)c3CC(=O)O)c3cc(-c4cccc5c(N)nccc45)ccc32)C1]

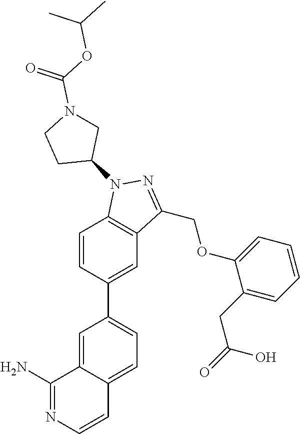 [SMILES: CC(C)OC(=O)N1CC[C@H](n2nc(COc3ccccc3CC(=O)O)c3cc(-c4ccc5ccnc(N)c5c4)ccc32)C1]

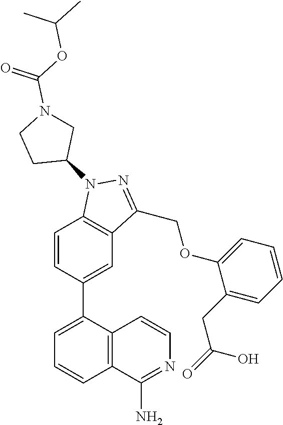 [SMILES: CC(C)OC(=O)N1CC[C@H](n2nc(COc3ccccc3CC(=O)O)c3cc(-c4cccc5c(N)nccc45)ccc32)C1]

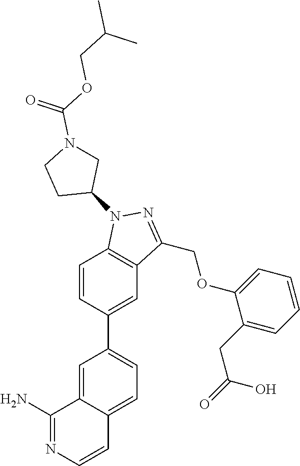 [SMILES: CC(C)COC(=O)N1CC[C@H](n2nc(COc3ccccc3CC(=O)O)c3cc(-c4ccc5ccnc(N)c5c4)ccc32)C1]

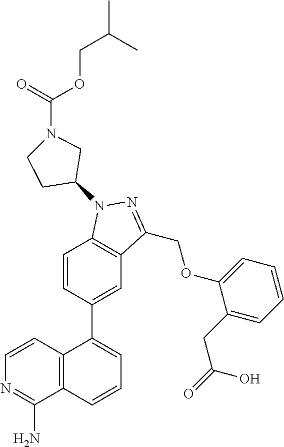 [SMILES: CC(C)COC(=O)N1CC[C@H](n2nc(COc3ccccc3CC(=O)O)c3cc(-c4cccc5c(N)nccc45)ccc32)C1]

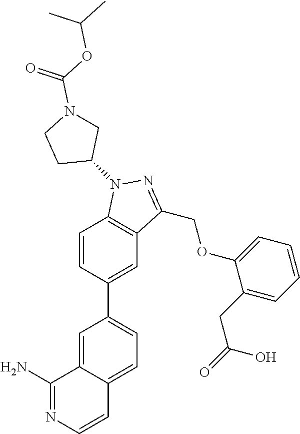 [SMILES: CC(C)OC(=O)N1CC[C@@H](n2nc(COc3ccccc3CC(=O)O)c3cc(-c4ccc5ccnc(N)c5c4)ccc32)C1]

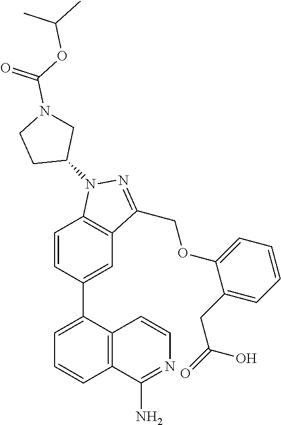 [SMILES: CC(C)OC(=O)N1CC[C@@H](n2nc(COc3ccccc3CC(=O)O)c3cc(-c4cccc5c(N)nccc45)ccc32)C1]

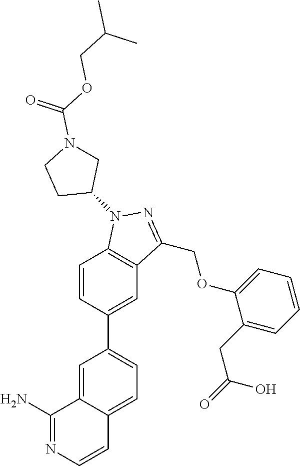 [SMILES: CC(C)COC(=O)N1CC[C@@H](n2nc(COc3ccccc3CC(=O)O)c3cc(-c4ccc5ccnc(N)c5c4)ccc32)C1]

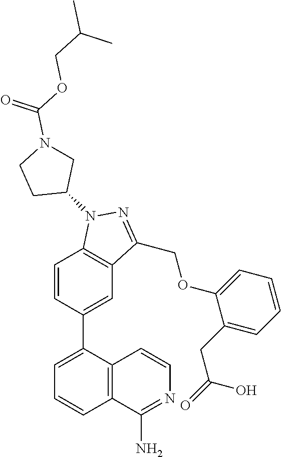 [SMILES: CC(C)COC(=O)N1CC[C@@H](n2nc(COc3ccccc3CC(=O)O)c3cc(-c4cccc5c(N)nccc45)ccc32)C1]

83. A compound of formula (III), or a pharmaceutically acceptable salt thereof: 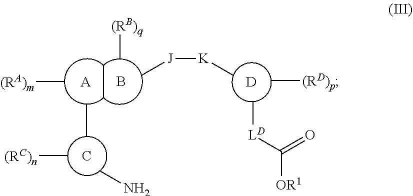
wherein:
ring 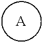
is arylene or heteroarylene;
ring 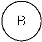
is cycloalkylene, heterocycloalkylene, cycloalkenylene, or heterocycloalkenylene;
ring 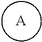
is fused to ring 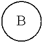
at two and only two adjacent positions;
ring 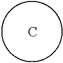
is a bicyclic ring system, where the ring attached to ring 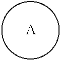
is aryl or heteroaryl;
ring 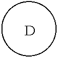
is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or 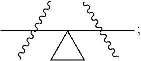 [SMILES: *C1(*)CC1]
K represents a bond, —O—, —NH—, —C(O)—, —CH₂—, —S—, —S(O)—, —SO₂—, —N(alkyl)-, —CH(alkyl)-, or —CH(cycloalkyl)-;
wherein at least one of J and K is a bond, —C(O)—, —CH₂—, —CH₂CH₂—, —CH(alkyl), or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))—, —CH(NH(cycloalkyl))—, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂N(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CH₂O(optionally substituted aryl), —C(O)O(alkyl), —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two adjacent occurrences of R^{B} taken together with the intervening atoms form an aromatic ring; or two geminal occurrences of R^{B} taken together with the atom to which they are attached form an optionally substituted spirocycloalkyl or a spiroheterocycloalkyl;
R^{C}, independently for each occurrence, represents H, halo, —OH, cyano, or amino, or is optionally substituted alkoxy, haloalkoxy, alkyl, cycloalkyl, heterocycloalkyl, or (heteroaryl)alkoxy;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, n, p, and q are each independently 0, 1, or 2.

84. The compound of claim 83, wherein ring 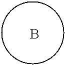
is cycloalkylene or heterocycloalkylene.

85. The compound of claim 83 or 84, wherein:
ring 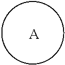
is arylene;
ring 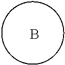
is cycloalkylene;
ring 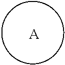
is fused to ring 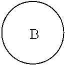
at two and only two adjacent positions;
ring 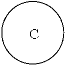
is a bicyclic ring system, where the ring attached to ring 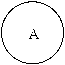
is aryl;
ring 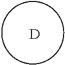
is aryl;
J represents —O—;
K represents a bond;
L^{D} represents —CH₂—;
R^{A}, independently for each occurrence, represents H, halo, alkyl, optionally substituted aryl, optionally substituted heteroaryl, or optionally substituted cycloalkyl;
R^{B}, independently for each occurrence, represents H, or two adjacent occurrences of R^{B} taken together with the intervening atoms form an aromatic ring; or two geminal occurrences of R^{B} taken together with the atom to which they are attached form an optionally substituted spirocycloalkyl or a spiroheterocycloalkyl;
R^{C} represents H;
R^{D} represents H or optionally substituted alkyl;
R¹ represents H.

86. The compound of any one of claims 83-85, having the structure of formula (IIIa):

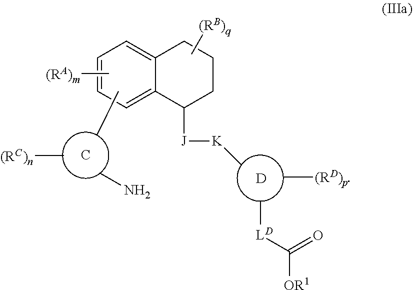

87. The compound of any one of claims 83-86, having the structure of formula (IIIa-1):

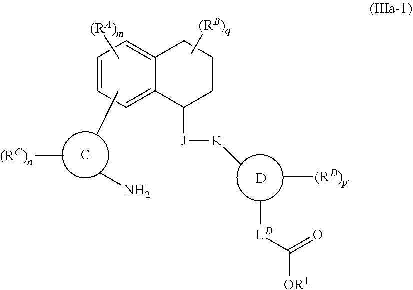

88. The compound of any one of claims 83-85, having the structure of formula (IIIb): 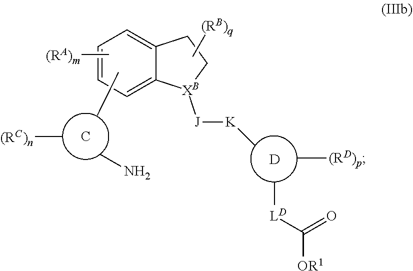
wherein X^{B} represents CH or N.

89. The compound of claim 88, having the structure of formula (IIIb-1): 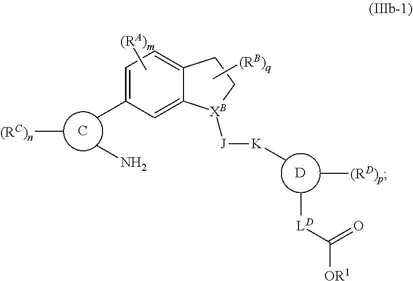
wherein X^{B} represents CH or N.

90. The compound of any one of claims 83-85, having the structure of formula (IIIc): 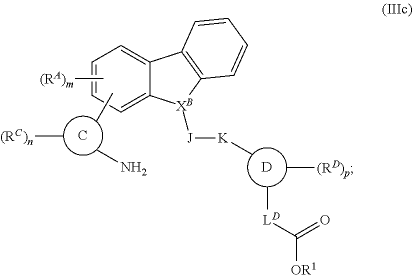
wherein X^{B} represents CH or N.

91. The compound of claim 90, having the structure of formula (IIIc-1): 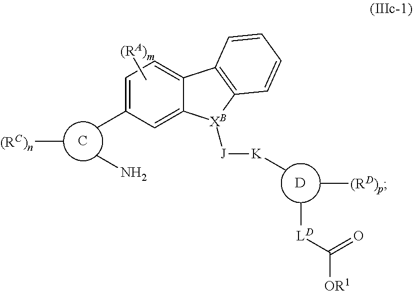
wherein X^{B} represents CH or N.

92. The compound of any one of claims 83-85, having the structure of formula (IIId):

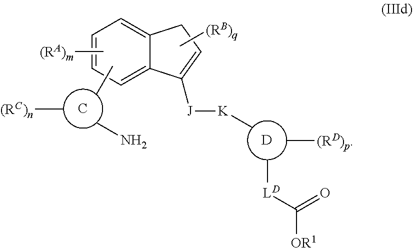

93. The compound of claim 92, having the structure of formula (IIId-1):

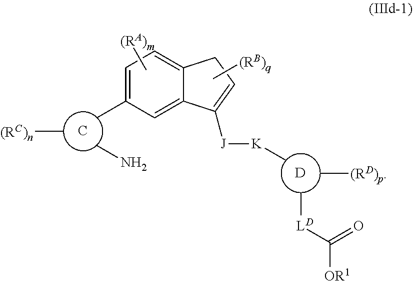

94. The compound of any one of claims 83-93, wherein ring 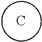
is a bicyclic ring system, where the ring attached to ring 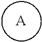
is aryl.

95. The compound of any one of claims 83-94, wherein 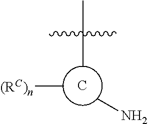
represents 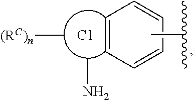
wherein ring 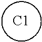
represents heteroaryl.

96. The compound of any one of claims 83-95, wherein 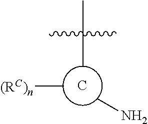
represents

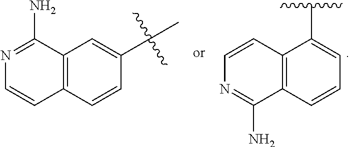 [SMILES: *c1ccc2ccnc(N)c2c1 ; *c1cccc2c(N)nccc12]

97. The compound of any one of claims 83-96, wherein R^{C} represents H.

98. The compound of any one of claims 83-84 and 86-97, wherein J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

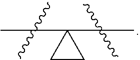 [SMILES: *C1(*)CC1]

99. The compound of any one of claims 83-98, wherein -J-K- represents —O—.

100. The compound of any one of claims 83-99, wherein ring 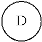
is aryl.

101. The compound of any one of claims 83-100, wherein 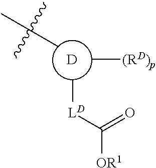
represents

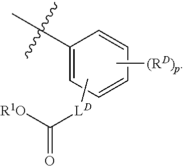

102. The compound of any one of claims 83-101, wherein 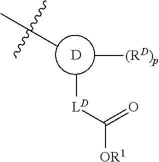
represents

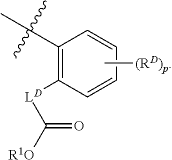

103. The compound of claim 83, wherein ring 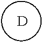
is bicyclic heteroaryl.

104. The compound of any one of claims 83-103, wherein R¹ is H.

105. The compound of any one of claims 83-104, wherein L^{D} represents —CH₂—.

106. The compound of any one of claims 83-105, wherein R^{D}, independently for each occurrence, represents H or alkyl.

107. The compound of any one of claims 83-106, wherein two geminal occurrences of R^{B} taken together with the atom to which they are attached form an optionally substituted spirocycloalkyl or a spiroheterocycloalkyl.

108. The compound of any one of claims 83-107, wherein two geminal occurrences of R^{B} taken together with the atom to which they are attached form a substituted spiroheterocycloalkyl.

109. The compound of any one of claims 83-108, wherein R^{B} represents H.

110. The compound of any one of claims 83-109, wherein R^{A}, independently for each occurrence, represents H, halo, alkyl, aryl, heteroaryl, or cycloalkyl.

111. The compound of any one of claims 83-110, wherein R^{A} represents H.

112. The compound of claim 83, selected from the following table:

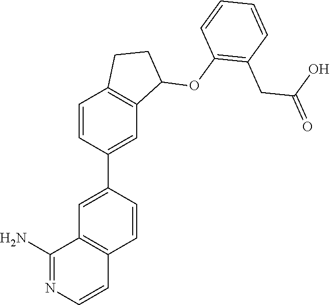 [SMILES: Nc1nccc2ccc(-c3ccc4c(c3)C(Oc3ccccc3CC(=O)O)CC4)cc12]

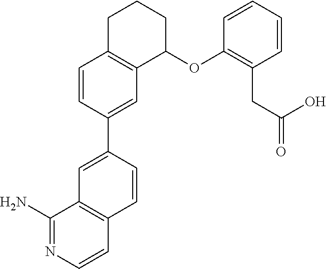 [SMILES: Nc1nccc2ccc(-c3ccc4c(c3)C(Oc3ccccc3CC(=O)O)CCC4)cc12]

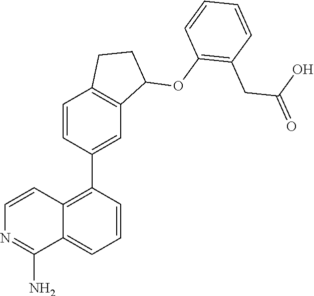 [SMILES: Nc1nccc2c(-c3ccc4c(c3)C(Oc3ccccc3CC(=O)O)CC4)cccc12]

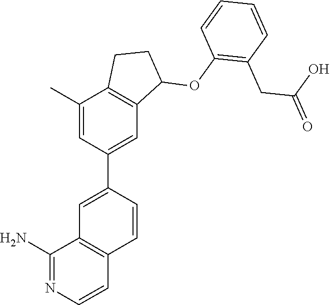 [SMILES: Cc1cc(-c2ccc3ccnc(N)c3c2)cc2c1CCC2Oc1ccccc1CC(=O)O]

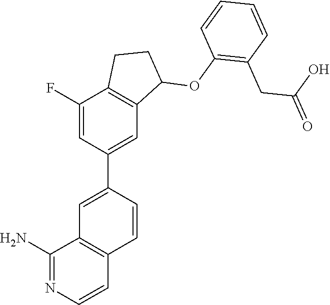 [SMILES: Nc1nccc2ccc(-c3cc(F)c4c(c3)C(Oc3ccccc3CC(=O)O)CC4)cc12]

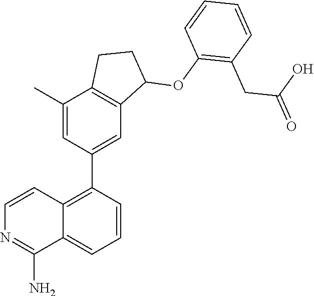 [SMILES: Cc1cc(-c2cccc3c(N)nccc23)cc2c1CCC2Oc1ccccc1CC(=O)O]

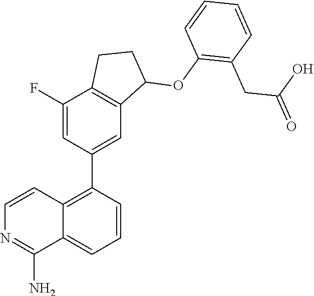 [SMILES: Nc1nccc2c(-c3cc(F)c4c(c3)C(Oc3ccccc3CC(=O)O)CC4)cccc12]

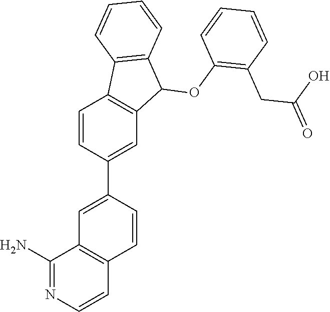 [SMILES: Nc1nccc2ccc(-c3ccc4c(c3)C(Oc3ccccc3CC(=O)O)c3ccccc3-4)cc12]

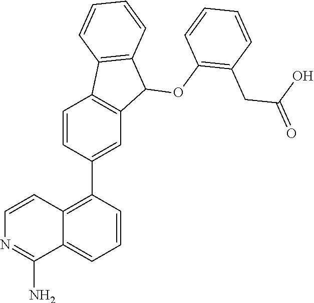 [SMILES: Nc1nccc2c(-c3ccc4c(c3)C(Oc3ccccc3CC(=O)O)c3ccccc3-4)cccc12]

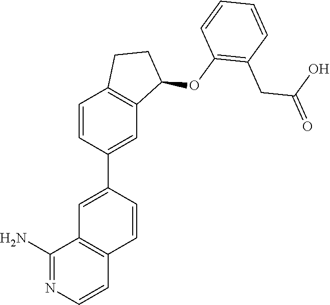 [SMILES: Nc1nccc2ccc(-c3ccc4c(c3)[C@H](Oc3ccccc3CC(=O)O)CC4)cc12]

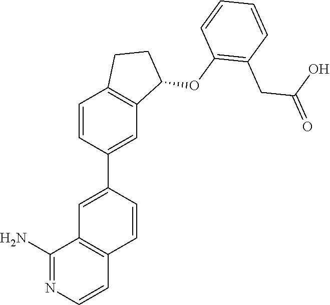 [SMILES: Nc1nccc2ccc(-c3ccc4c(c3)[C@@H](Oc3ccccc3CC(=O)O)CC4)cc12]

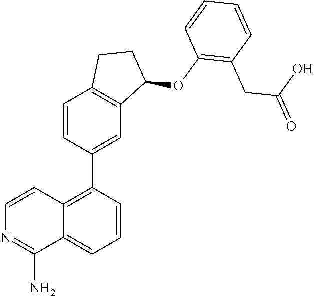 [SMILES: Nc1nccc2c(-c3ccc4c(c3)[C@H](Oc3ccccc3CC(=O)O)CC4)cccc12]

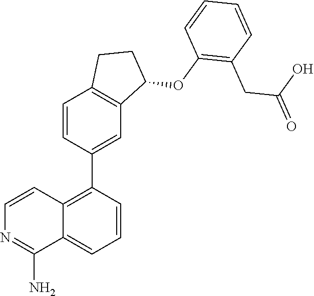 [SMILES: Nc1nccc2c(-c3ccc4c(c3)[C@@H](Oc3ccccc3CC(=O)O)CC4)cccc12]

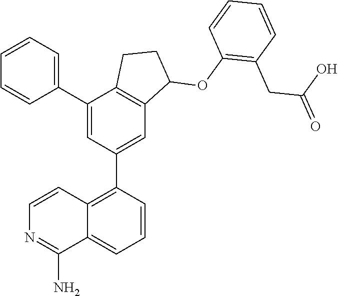 [SMILES: Nc1nccc2c(-c3cc(-c4ccccc4)c4c(c3)C(Oc3ccccc3CC(=O)O)CC4)cccc12]

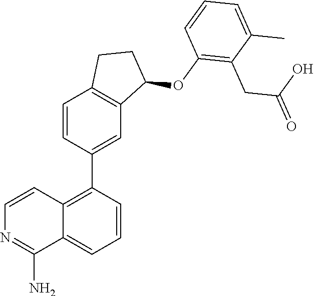 [SMILES: Cc1cccc(O[C@@H]2CCc3ccc(-c4cccc5c(N)nccc45)cc32)c1CC(=O)O]

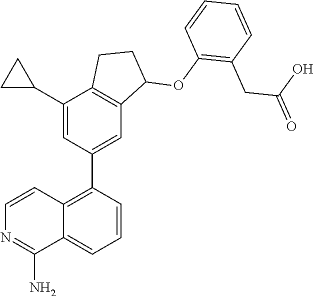 [SMILES: Nc1nccc2c(-c3cc(C4CC4)c4c(c3)C(Oc3ccccc3CC(=O)O)CC4)cccc12]

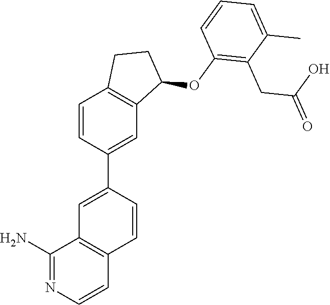 [SMILES: Cc1cccc(O[C@@H]2CCc3ccc(-c4ccc5ccnc(N)c5c4)cc32)c1CC(=O)O]

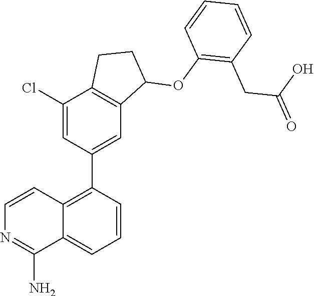 [SMILES: Nc1nccc2c(-c3cc(Cl)c4c(c3)C(Oc3ccccc3CC(=O)O)CC4)cccc12]

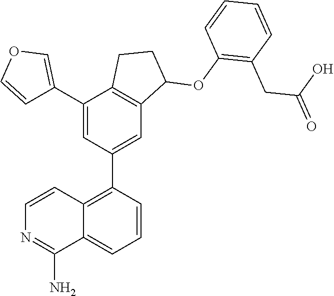 [SMILES: Nc1nccc2c(-c3cc(-c4ccoc4)c4c(c3)C(Oc3ccccc3CC(=O)O)CC4)cccc12]

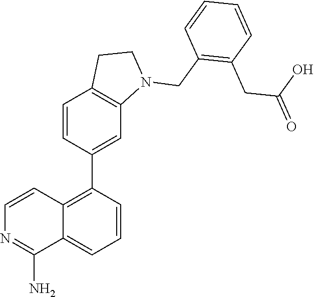 [SMILES: Nc1nccc2c(-c3ccc4c(c3)N(Cc3ccccc3CC(=O)O)CC4)cccc12]

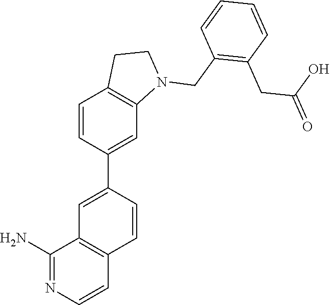 [SMILES: Nc1nccc2ccc(-c3ccc4c(c3)N(Cc3ccccc3CC(=O)O)CC4)cc12]

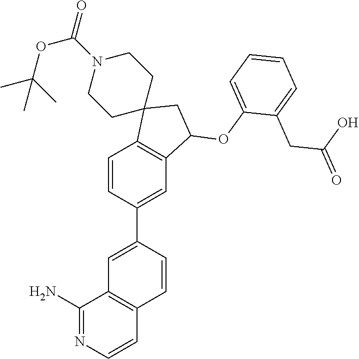 [SMILES: CC(C)(C)OC(=O)N1CCC2(CC1)CC(Oc1ccccc1CC(=O)O)c1cc(-c3ccc4ccnc(N)c4c3)ccc12]

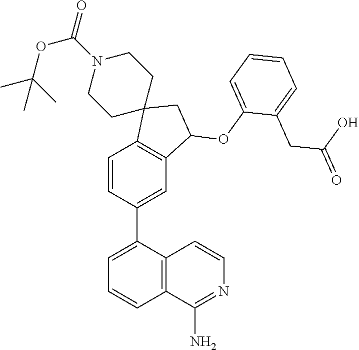 [SMILES: CC(C)(C)OC(=O)N1CCC2(CC1)CC(Oc1ccccc1CC(=O)O)c1cc(-c3cccc4c(N)nccc34)ccc12]

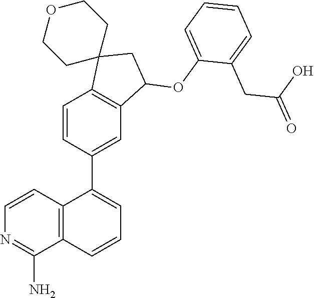 [SMILES: Nc1nccc2c(-c3ccc4c(c3)C(Oc3ccccc3CC(=O)O)CC43CCOCC3)cccc12]

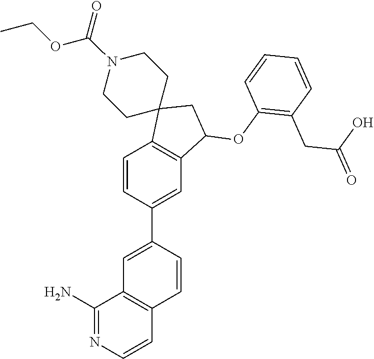 [SMILES: CCOC(=O)N1CCC2(CC1)CC(Oc1ccccc1CC(=O)O)c1cc(-c3ccc4ccnc(N)c4c3)ccc12]

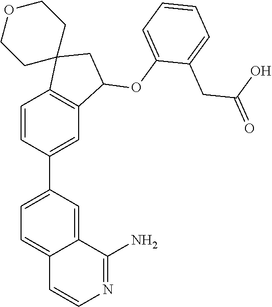 [SMILES: Nc1nccc2ccc(-c3ccc4c(c3)C(Oc3ccccc3CC(=O)O)CC43CCOCC3)cc12]

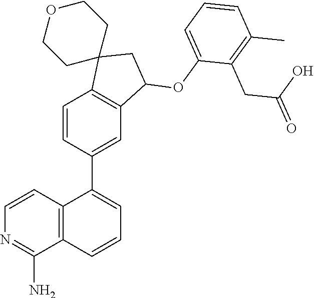 [SMILES: Cc1cccc(OC2CC3(CCOCC3)c3ccc(-c4cccc5c(N)nccc45)cc32)c1CC(=O)O]

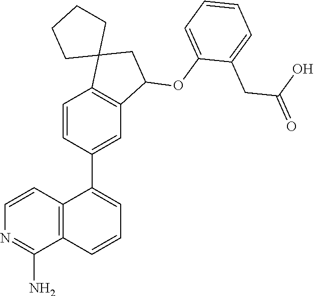 [SMILES: Nc1nccc2c(-c3ccc4c(c3)C(Oc3ccccc3CC(=O)O)CC43CCCC3)cccc12]

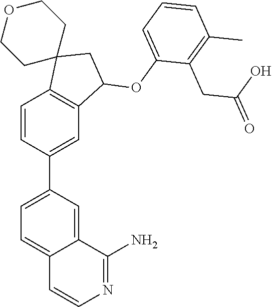 [SMILES: Cc1cccc(OC2CC3(CCOCC3)c3ccc(-c4ccc5ccnc(N)c5c4)cc32)c1CC(=O)O]

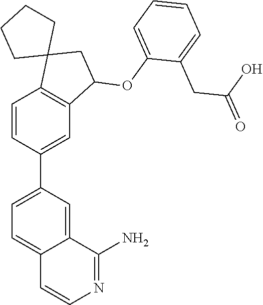 [SMILES: Nc1nccc2ccc(-c3ccc4c(c3)C(Oc3ccccc3CC(=O)O)CC43CCCC3)cc12]

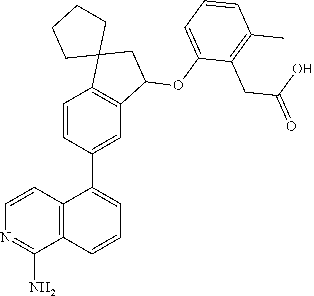 [SMILES: Cc1cccc(OC2CC3(CCCC3)c3ccc(-c4cccc5c(N)nccc45)cc32)c1CC(=O)O]

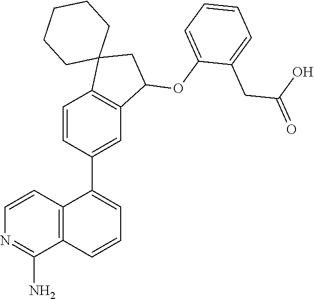 [SMILES: Nc1nccc2c(-c3ccc4c(c3)C(Oc3ccccc3CC(=O)O)CC43CCCCC3)cccc12]

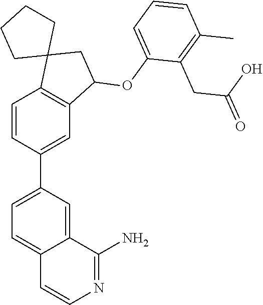 [SMILES: Cc1cccc(OC2CC3(CCCC3)c3ccc(-c4ccc5ccnc(N)c5c4)cc32)c1CC(=O)O]

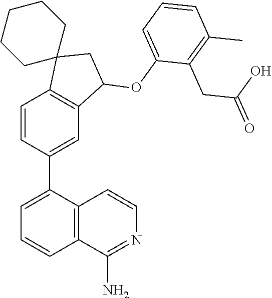 [SMILES: Cc1cccc(OC2CC3(CCCCC3)c3ccc(-c4cccc5c(N)nccc45)cc32)c1CC(=O)O]

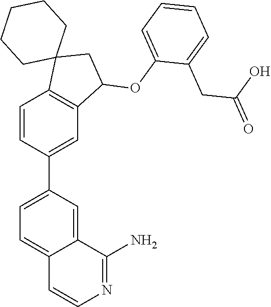 [SMILES: Nc1nccc2ccc(-c3ccc4c(c3)C(Oc3ccccc3CC(=O)O)CC43CCCCC3)cc12]

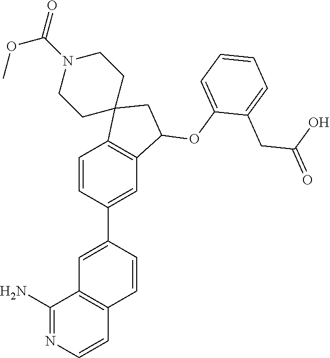 [SMILES: COC(=O)N1CCC2(CC1)CC(Oc1ccccc1CC(=O)O)c1cc(-c3ccc4ccnc(N)c4c3)ccc12]

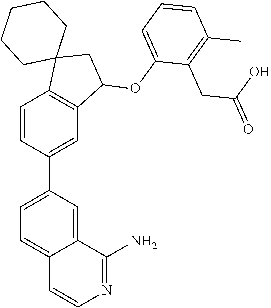 [SMILES: Cc1cccc(OC2CC3(CCCCC3)c3ccc(-c4ccc5ccnc(N)c5c4)cc32)c1CC(=O)O]

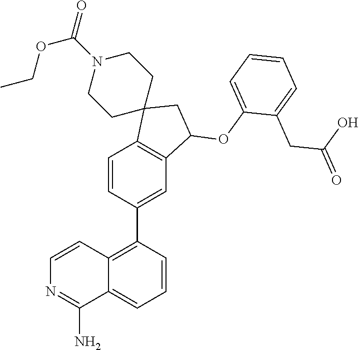 [SMILES: CCOC(=O)N1CCC2(CC1)CC(Oc1ccccc1CC(=O)O)c1cc(-c3cccc4c(N)nccc34)ccc12]

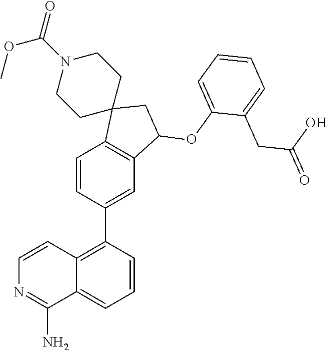 [SMILES: COC(=O)N1CCC2(CC1)CC(Oc1ccccc1CC(=O)O)c1cc(-c3cccc4c(N)nccc34)ccc12]

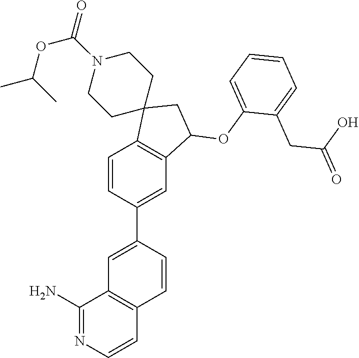 [SMILES: CC(C)OC(=O)N1CCC2(CC1)CC(Oc1ccccc1CC(=O)O)c1cc(-c3ccc4ccnc(N)c4c3)ccc12]

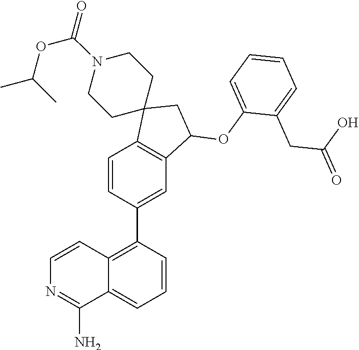 [SMILES: CC(C)OC(=O)N1CCC2(CC1)CC(Oc1ccccc1CC(=O)O)c1cc(-c3cccc4c(N)nccc34)ccc12]

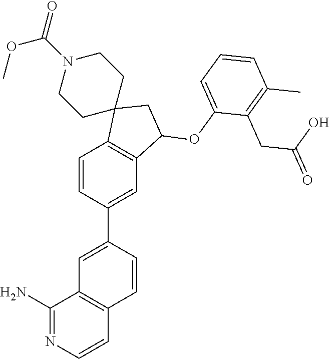 [SMILES: COC(=O)N1CCC2(CC1)CC(Oc1cccc(C)c1CC(=O)O)c1cc(-c3ccc4ccnc(N)c4c3)ccc12]

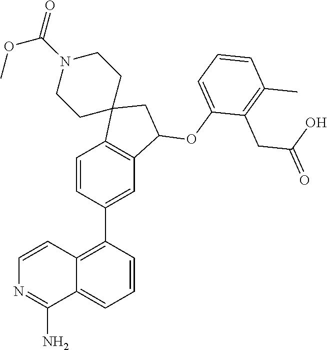 [SMILES: COC(=O)N1CCC2(CC1)CC(Oc1cccc(C)c1CC(=O)O)c1cc(-c3cccc4c(N)nccc34)ccc12]

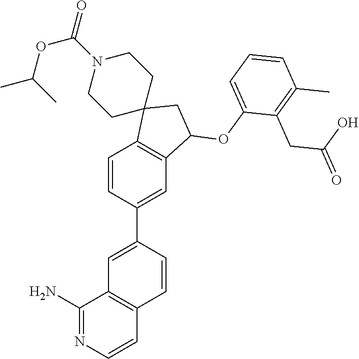 [SMILES: Cc1cccc(OC2CC3(CCN(C(=O)OC(C)C)CC3)c3ccc(-c4ccc5ccnc(N)c5c4)cc32)c1CC(=O)O]

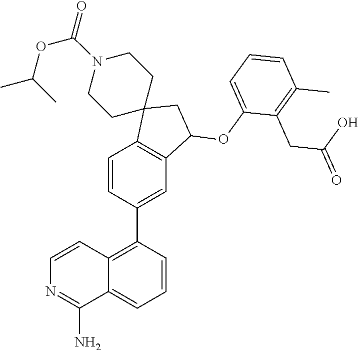 [SMILES: Cc1cccc(OC2CC3(CCN(C(=O)OC(C)C)CC3)c3ccc(-c4cccc5c(N)nccc45)cc32)c1CC(=O)O]

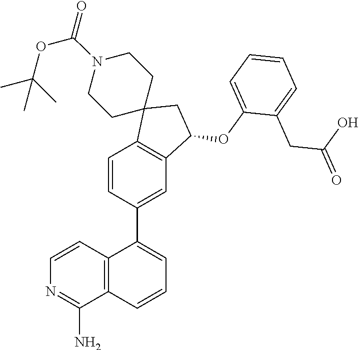 [SMILES: CC(C)(C)OC(=O)N1CCC2(CC1)C[C@H](Oc1ccccc1CC(=O)O)c1cc(-c3cccc4c(N)nccc34)ccc12]

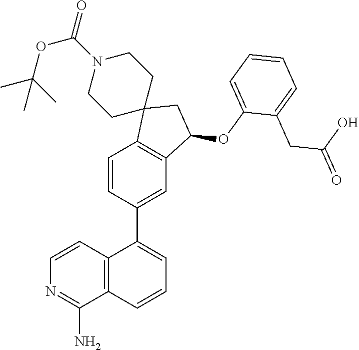 [SMILES: CC(C)(C)OC(=O)N1CCC2(CC1)C[C@@H](Oc1ccccc1CC(=O)O)c1cc(-c3cccc4c(N)nccc34)ccc12]

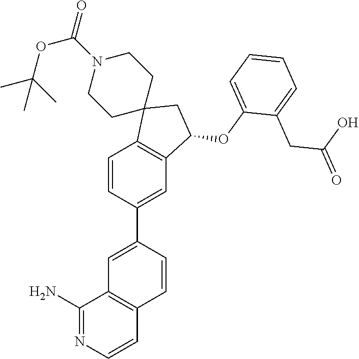 [SMILES: CC(C)(C)OC(=O)N1CCC2(CC1)C[C@H](Oc1ccccc1CC(=O)O)c1cc(-c3ccc4ccnc(N)c4c3)ccc12]

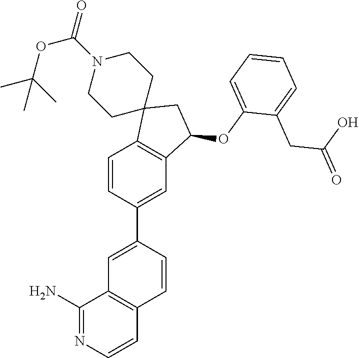 [SMILES: CC(C)(C)OC(=O)N1CCC2(CC1)C[C@@H](Oc1ccccc1CC(=O)O)c1cc(-c3ccc4ccnc(N)c4c3)ccc12]

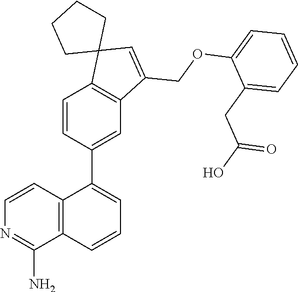 [SMILES: Nc1nccc2c(-c3ccc4c(c3)C(COc3ccccc3CC(=O)O)=CC43CCCC3)cccc12]

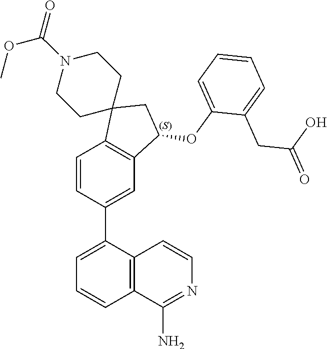

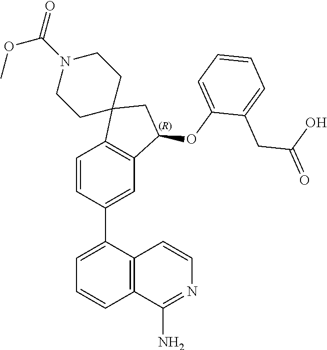

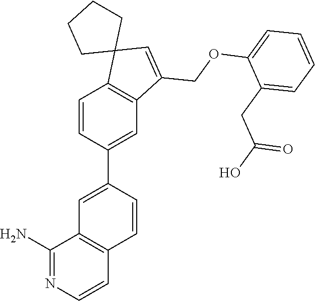 [SMILES: Nc1nccc2ccc(-c3ccc4c(c3)C(COc3ccccc3CC(=O)O)=CC43CCCC3)cc12]

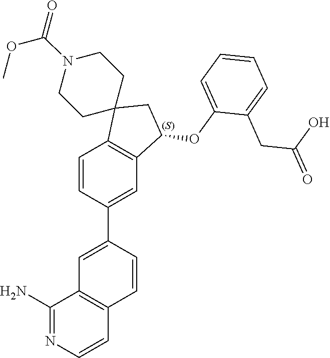

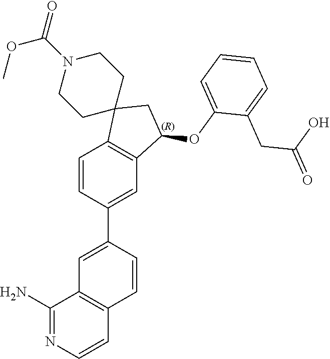

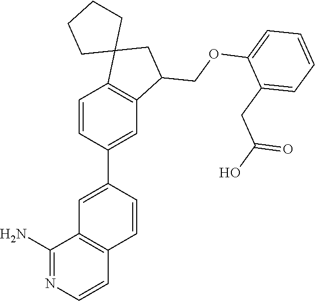 [SMILES: Nc1nccc2ccc(-c3ccc4c(c3)C(COc3ccccc3CC(=O)O)CC43CCCC3)cc12]

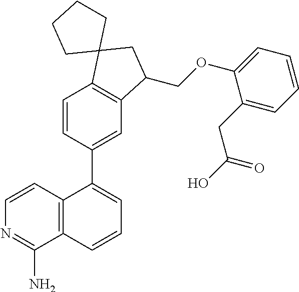 [SMILES: Nc1nccc2c(-c3ccc4c(c3)C(COc3ccccc3CC(=O)O)CC43CCCC3)cccc12]

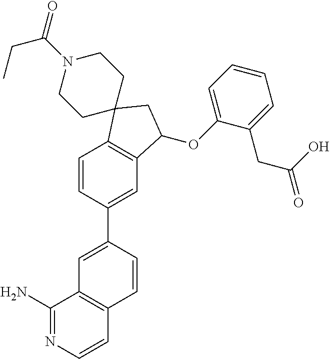 [SMILES: CCC(=O)N1CCC2(CC1)CC(Oc1ccccc1CC(=O)O)c1cc(-c3ccc4ccnc(N)c4c3)ccc12]

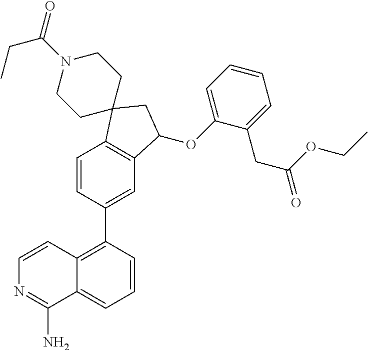 [SMILES: CCOC(=O)Cc1ccccc1OC1CC2(CCN(C(=O)CC)CC2)c2ccc(-c3cccc4c(N)nccc34)cc21]

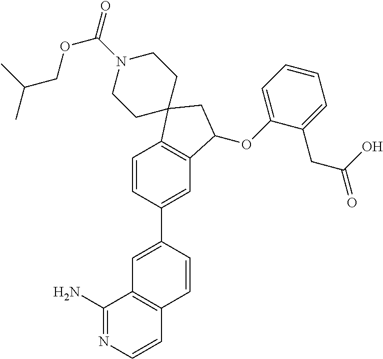 [SMILES: CC(C)COC(=O)N1CCC2(CC1)CC(Oc1ccccc1CC(=O)O)c1cc(-c3ccc4ccnc(N)c4c3)ccc12]

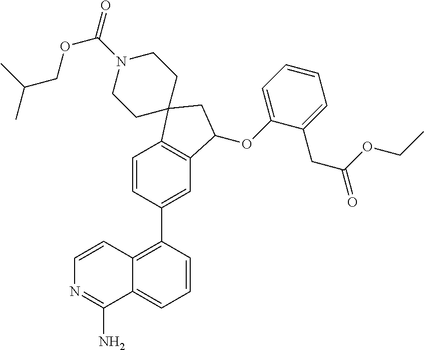 [SMILES: CCOC(=O)Cc1ccccc1OC1CC2(CCN(C(=O)OCC(C)C)CC2)c2ccc(-c3cccc4c(N)nccc34)cc21]

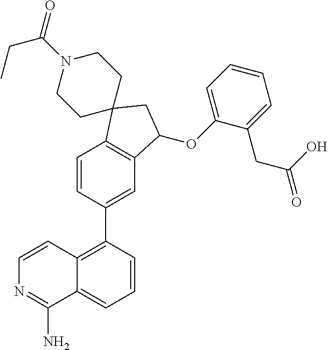 [SMILES: CCC(=O)N1CCC2(CC1)CC(Oc1ccccc1CC(=O)O)c1cc(-c3cccc4c(N)nccc34)ccc12]

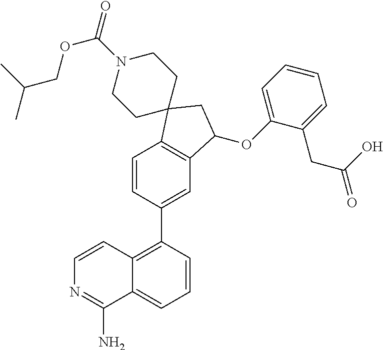 [SMILES: CC(C)COC(=O)N1CCC2(CC1)CC(Oc1ccccc1CC(=O)O)c1cc(-c3cccc4c(N)nccc34)ccc12]

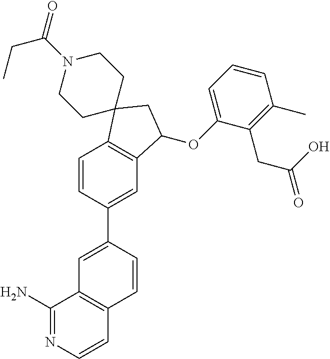 [SMILES: CCC(=O)N1CCC2(CC1)CC(Oc1cccc(C)c1CC(=O)O)c1cc(-c3ccc4ccnc(N)c4c3)ccc12]

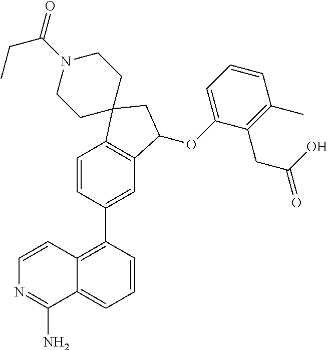 [SMILES: CCC(=O)N1CCC2(CC1)CC(Oc1cccc(C)c1CC(=O)O)c1cc(-c3cccc4c(N)nccc34)ccc12]

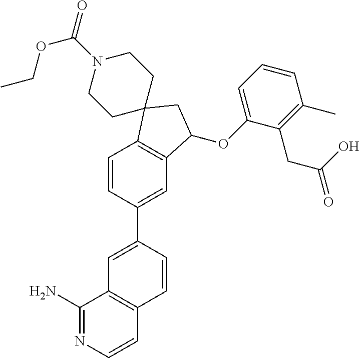 [SMILES: CCOC(=O)N1CCC2(CC1)CC(Oc1cccc(C)c1CC(=O)O)c1cc(-c3ccc4ccnc(N)c4c3)ccc12]

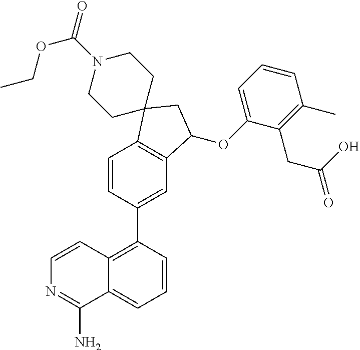 [SMILES: CCOC(=O)N1CCC2(CC1)CC(Oc1cccc(C)c1CC(=O)O)c1cc(-c3cccc4c(N)nccc34)ccc12]

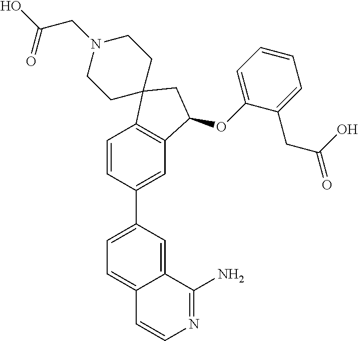 [SMILES: Nc1nccc2ccc(-c3ccc4c(c3)[C@H](Oc3ccccc3CC(=O)O)CC43CCN(CC(=O)O)CC3)cc12]

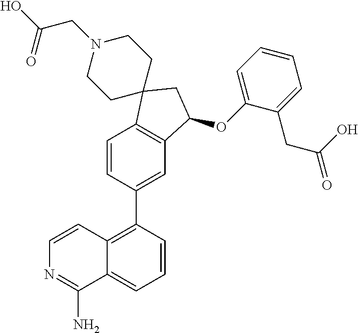 [SMILES: Nc1nccc2c(-c3ccc4c(c3)[C@H](Oc3ccccc3CC(=O)O)CC43CCN(CC(=O)O)CC3)cccc12]

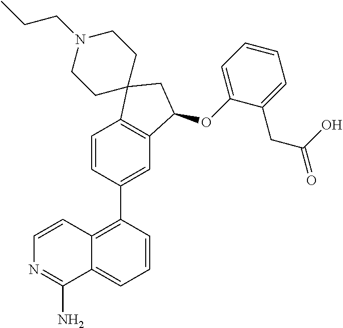 [SMILES: CCCN1CCC2(CC1)C[C@@H](Oc1ccccc1CC(=O)O)c1cc(-c3cccc4c(N)nccc34)ccc12]

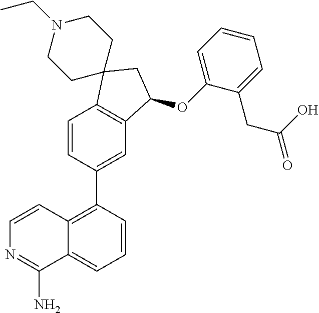 [SMILES: CCN1CCC2(CC1)C[C@@H](Oc1ccccc1CC(=O)O)c1cc(-c3cccc4c(N)nccc34)ccc12]

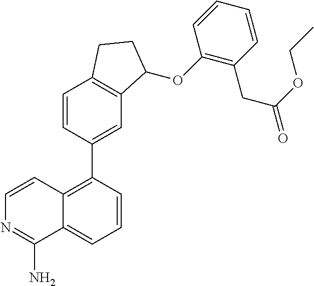 [SMILES: CCOC(=O)Cc1ccccc1OC1CCc2ccc(-c3cccc4c(N)nccc34)cc21]

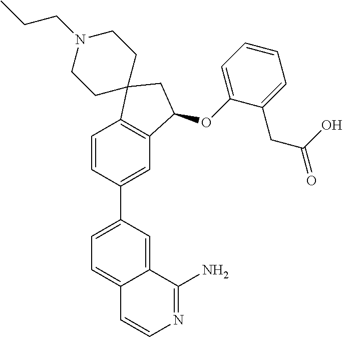 [SMILES: CCCN1CCC2(CC1)C[C@@H](Oc1ccccc1CC(=O)O)c1cc(-c3ccc4ccnc(N)c4c3)ccc12]

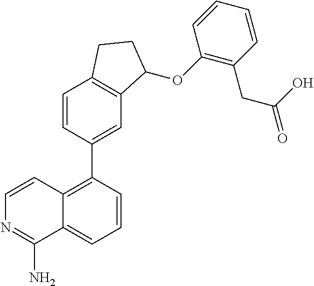 [SMILES: Nc1nccc2c(-c3ccc4c(c3)C(Oc3ccccc3CC(=O)O)CC4)cccc12]

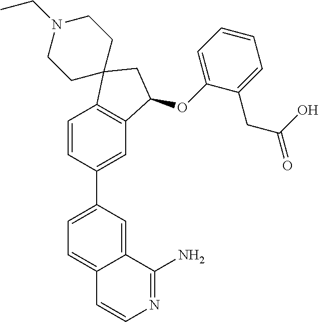 [SMILES: CCN1CCC2(CC1)C[C@@H](Oc1ccccc1CC(=O)O)c1cc(-c3ccc4ccnc(N)c4c3)ccc12]

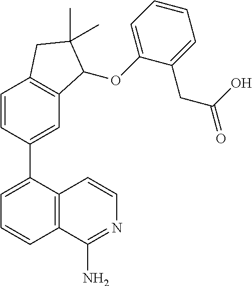 [SMILES: CC1(C)Cc2ccc(-c3cccc4c(N)nccc34)cc2C1Oc1ccccc1CC(=O)O]

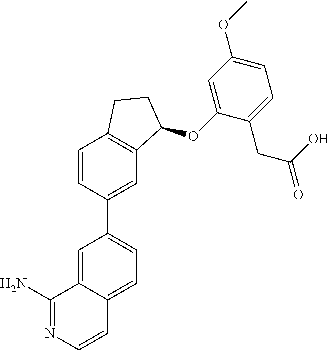 [SMILES: COc1ccc(CC(=O)O)c(O[C@@H]2CCc3ccc(-c4ccc5ccnc(N)c5c4)cc32)c1]

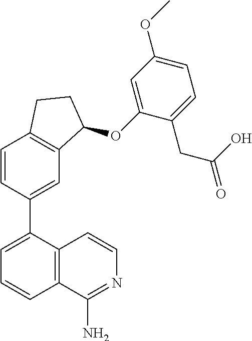 [SMILES: COc1ccc(CC(=O)O)c(O[C@@H]2CCc3ccc(-c4cccc5c(N)nccc45)cc32)c1]

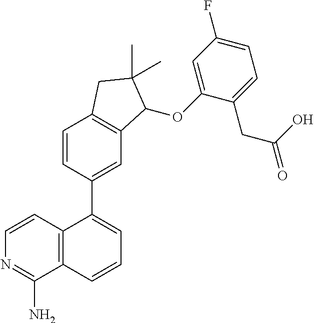 [SMILES: CC1(C)Cc2ccc(-c3cccc4c(N)nccc34)cc2C1Oc1cc(F)ccc1CC(=O)O]

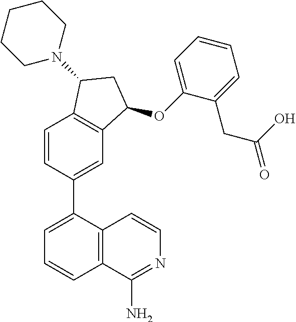 [SMILES: Nc1nccc2c(-c3ccc4c(c3)[C@H](Oc3ccccc3CC(=O)O)C[C@H]4N3CCCCC3)cccc12]

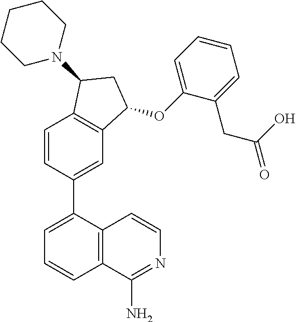 [SMILES: Nc1nccc2c(-c3ccc4c(c3)[C@@H](Oc3ccccc3CC(=O)O)C[C@@H]4N3CCCCC3)cccc12]

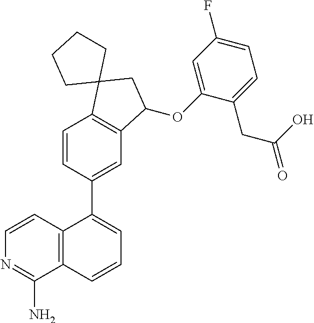 [SMILES: Nc1nccc2c(-c3ccc4c(c3)C(Oc3cc(F)ccc3CC(=O)O)CC43CCCC3)cccc12]

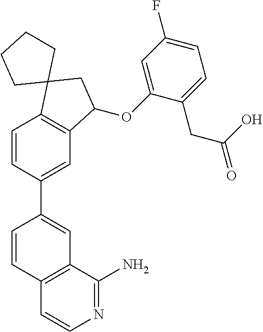 [SMILES: Nc1nccc2ccc(-c3ccc4c(c3)C(Oc3cc(F)ccc3CC(=O)O)CC43CCCC3)cc12]

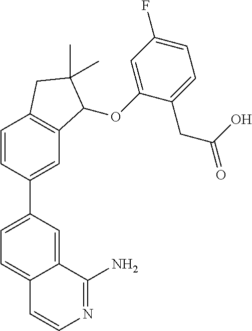 [SMILES: CC1(C)Cc2ccc(-c3ccc4ccnc(N)c4c3)cc2C1Oc1cc(F)ccc1CC(=O)O]

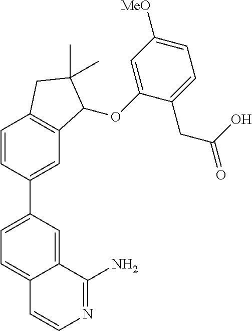 [SMILES: COc1ccc(CC(=O)O)c(OC2c3cc(-c4ccc5ccnc(N)c5c4)ccc3CC2(C)C)c1]

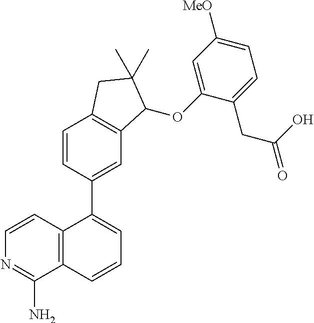 [SMILES: COc1ccc(CC(=O)O)c(OC2c3cc(-c4cccc5c(N)nccc45)ccc3CC2(C)C)c1]

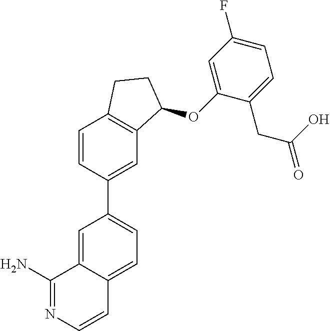 [SMILES: Nc1nccc2ccc(-c3ccc4c(c3)[C@H](Oc3cc(F)ccc3CC(=O)O)CC4)cc12]

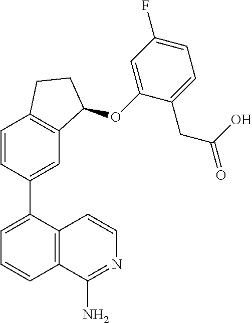 [SMILES: Nc1nccc2c(-c3ccc4c(c3)[C@H](Oc3cc(F)ccc3CC(=O)O)CC4)cccc12]

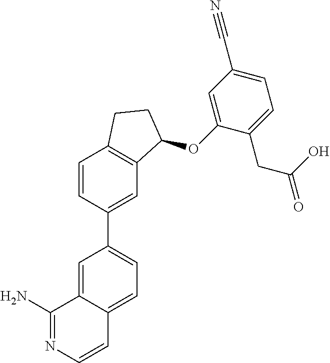 [SMILES: N#Cc1ccc(CC(=O)O)c(O[C@@H]2CCc3ccc(-c4ccc5ccnc(N)c5c4)cc32)c1]

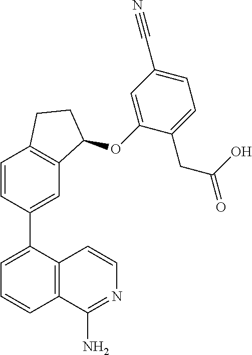 [SMILES: N#Cc1ccc(CC(=O)O)c(O[C@@H]2CCc3ccc(-c4cccc5c(N)nccc45)cc32)c1]

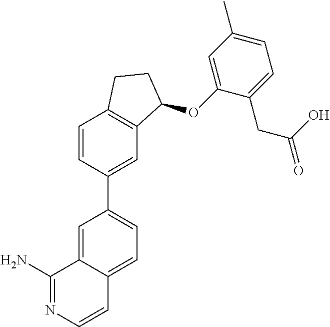 [SMILES: Cc1ccc(CC(=O)O)c(O[C@@H]2CCc3ccc(-c4ccc5ccnc(N)c5c4)cc32)c1]

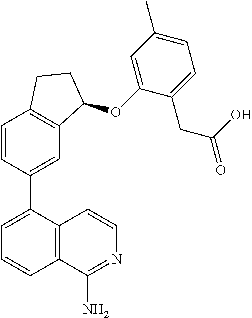 [SMILES: Cc1ccc(CC(=O)O)c(O[C@@H]2CCc3ccc(-c4cccc5c(N)nccc45)cc32)c1]

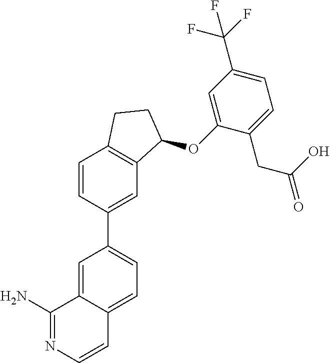 [SMILES: Nc1nccc2ccc(-c3ccc4c(c3)[C@H](Oc3cc(C(F)(F)F)ccc3CC(=O)O)CC4)cc12]

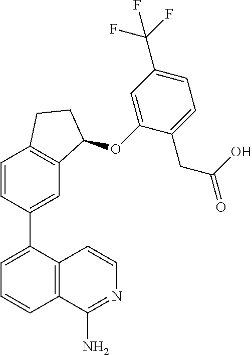 [SMILES: Nc1nccc2c(-c3ccc4c(c3)[C@H](Oc3cc(C(F)(F)F)ccc3CC(=O)O)CC4)cccc12]

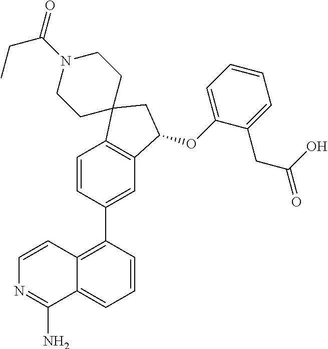 [SMILES: CCC(=O)N1CCC2(CC1)C[C@H](Oc1ccccc1CC(=O)O)c1cc(-c3cccc4c(N)nccc34)ccc12]

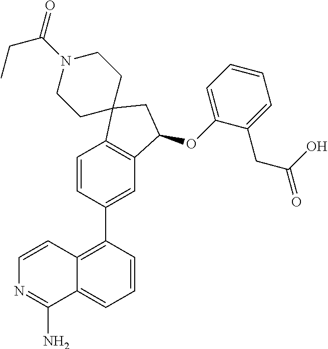 [SMILES: CCC(=O)N1CCC2(CC1)C[C@@H](Oc1ccccc1CC(=O)O)c1cc(-c3cccc4c(N)nccc34)ccc12]

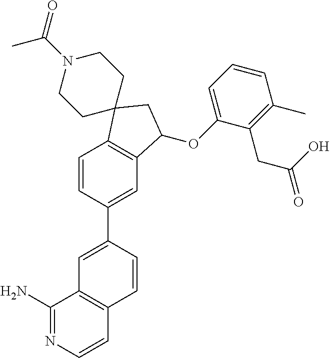 [SMILES: CC(=O)N1CCC2(CC1)CC(Oc1cccc(C)c1CC(=O)O)c1cc(-c3ccc4ccnc(N)c4c3)ccc12]

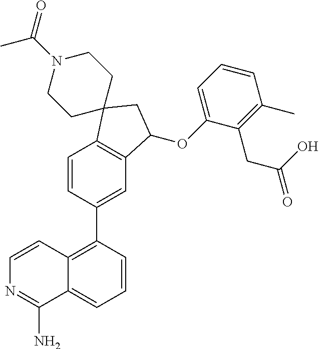 [SMILES: CC(=O)N1CCC2(CC1)CC(Oc1cccc(C)c1CC(=O)O)c1cc(-c3cccc4c(N)nccc34)ccc12]

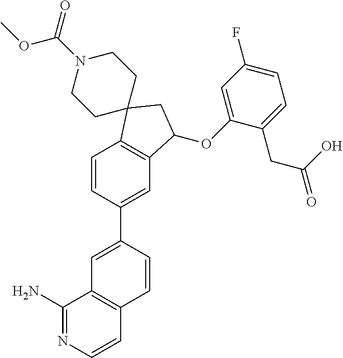 [SMILES: COC(=O)N1CCC2(CC1)CC(Oc1cc(F)ccc1CC(=O)O)c1cc(-c3ccc4ccnc(N)c4c3)ccc12]

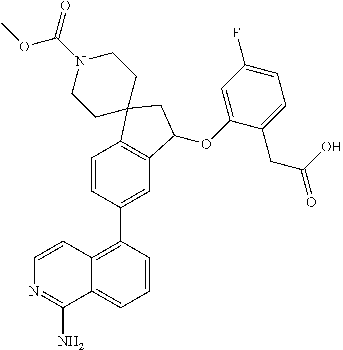 [SMILES: COC(=O)N1CCC2(CC1)CC(Oc1cc(F)ccc1CC(=O)O)c1cc(-c3cccc4c(N)nccc34)ccc12]

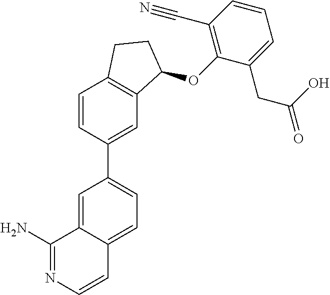 [SMILES: N#Cc1cccc(CC(=O)O)c1O[C@@H]1CCc2ccc(-c3ccc4ccnc(N)c4c3)cc21]

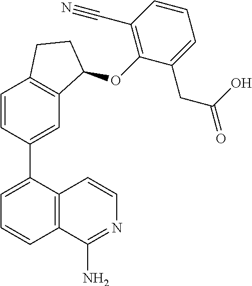 [SMILES: N#Cc1cccc(CC(=O)O)c1O[C@@H]1CCc2ccc(-c3cccc4c(N)nccc34)cc21]

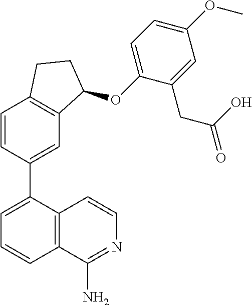 [SMILES: COc1ccc(O[C@@H]2CCc3ccc(-c4cccc5c(N)nccc45)cc32)c(CC(=O)O)c1]

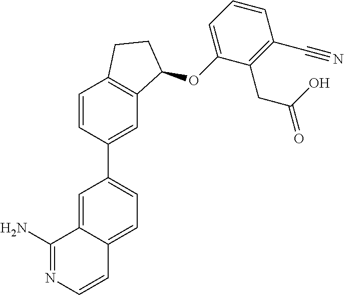 [SMILES: N#Cc1cccc(O[C@@H]2CCc3ccc(-c4ccc5ccnc(N)c5c4)cc32)c1CC(=O)O]

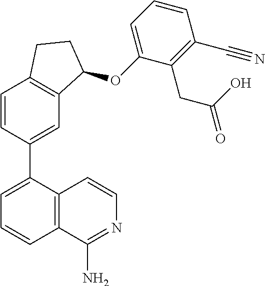 [SMILES: N#Cc1cccc(O[C@@H]2CCc3ccc(-c4cccc5c(N)nccc45)cc32)c1CC(=O)O]

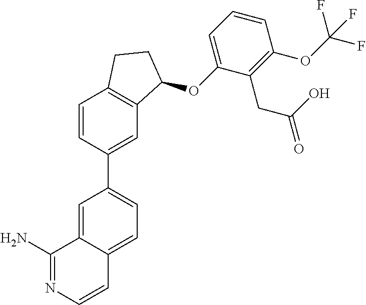 [SMILES: Nc1nccc2ccc(-c3ccc4c(c3)[C@H](Oc3cccc(OC(F)(F)F)c3CC(=O)O)CC4)cc12]

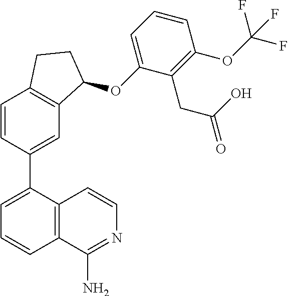 [SMILES: Nc1nccc2c(-c3ccc4c(c3)[C@H](Oc3cccc(OC(F)(F)F)c3CC(=O)O)CC4)cccc12]

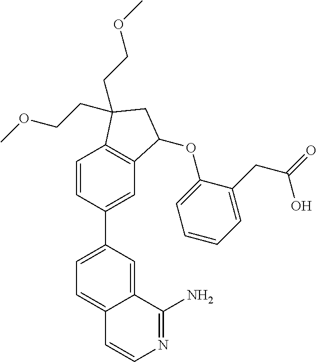 [SMILES: COCCC1(CCOC)CC(Oc2ccccc2CC(=O)O)c2cc(-c3ccc4ccnc(N)c4c3)ccc21]

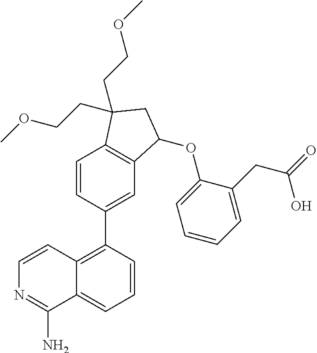 [SMILES: COCCC1(CCOC)CC(Oc2ccccc2CC(=O)O)c2cc(-c3cccc4c(N)nccc34)ccc21]

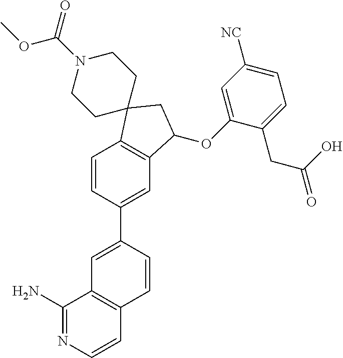 [SMILES: COC(=O)N1CCC2(CC1)CC(Oc1cc(C#N)ccc1CC(=O)O)c1cc(-c3ccc4ccnc(N)c4c3)ccc12]

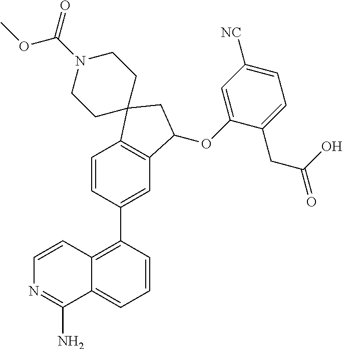 [SMILES: COC(=O)N1CCC2(CC1)CC(Oc1cc(C#N)ccc1CC(=O)O)c1cc(-c3cccc4c(N)nccc34)ccc12]

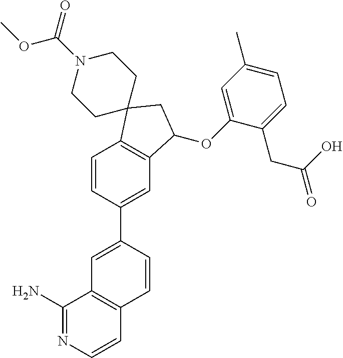 [SMILES: COC(=O)N1CCC2(CC1)CC(Oc1cc(C)ccc1CC(=O)O)c1cc(-c3ccc4ccnc(N)c4c3)ccc12]

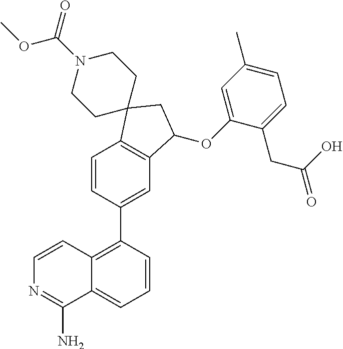 [SMILES: COC(=O)N1CCC2(CC1)CC(Oc1cc(C)ccc1CC(=O)O)c1cc(-c3cccc4c(N)nccc34)ccc12]

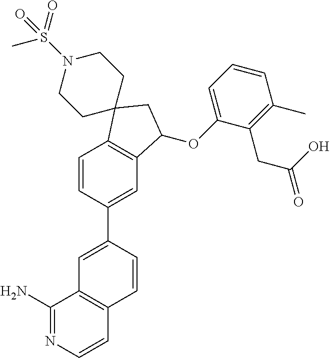 [SMILES: Cc1cccc(OC2CC3(CCN(S(C)(=O)=O)CC3)c3ccc(-c4ccc5ccnc(N)c5c4)cc32)c1CC(=O)O]

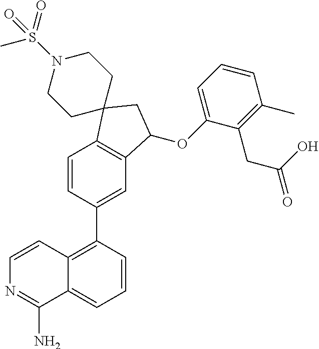 [SMILES: Cc1cccc(OC2CC3(CCN(S(C)(=O)=O)CC3)c3ccc(-c4cccc5c(N)nccc45)cc32)c1CC(=O)O]

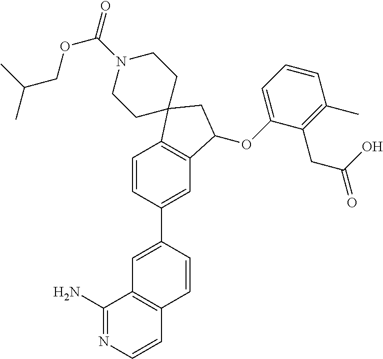 [SMILES: Cc1cccc(OC2CC3(CCN(C(=O)OCC(C)C)CC3)c3ccc(-c4ccc5ccnc(N)c5c4)cc32)c1CC(=O)O]

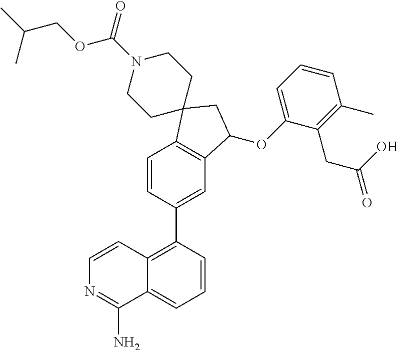 [SMILES: Cc1cccc(OC2CC3(CCN(C(=O)OCC(C)C)CC3)c3ccc(-c4cccc5c(N)nccc45)cc32)c1CC(=O)O]

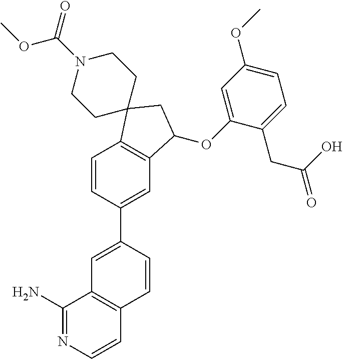 [SMILES: COC(=O)N1CCC2(CC1)CC(Oc1cc(OC)ccc1CC(=O)O)c1cc(-c3ccc4ccnc(N)c4c3)ccc12]

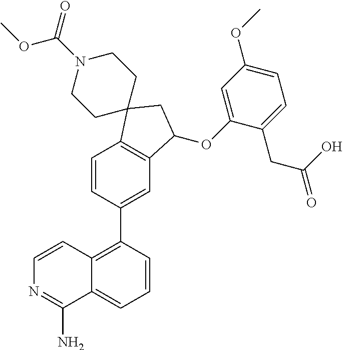 [SMILES: COC(=O)N1CCC2(CC1)CC(Oc1cc(OC)ccc1CC(=O)O)c1cc(-c3cccc4c(N)nccc34)ccc12]

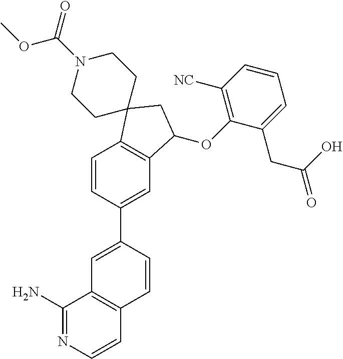 [SMILES: COC(=O)N1CCC2(CC1)CC(Oc1c(C#N)cccc1CC(=O)O)c1cc(-c3ccc4ccnc(N)c4c3)ccc12]

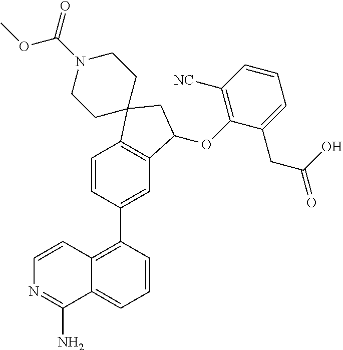 [SMILES: COC(=O)N1CCC2(CC1)CC(Oc1c(C#N)cccc1CC(=O)O)c1cc(-c3cccc4c(N)nccc34)ccc12]

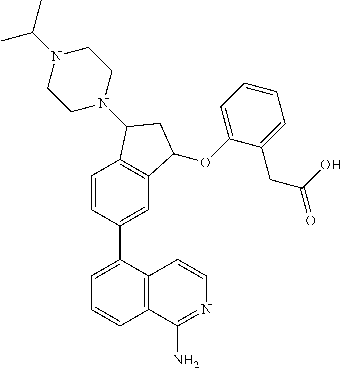 [SMILES: CC(C)N1CCN(C2CC(Oc3ccccc3CC(=O)O)c3cc(-c4cccc5c(N)nccc45)ccc32)CC1]

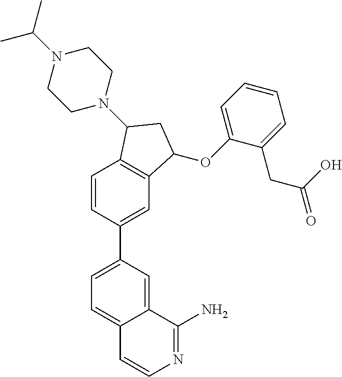 [SMILES: CC(C)N1CCN(C2CC(Oc3ccccc3CC(=O)O)c3cc(-c4ccc5ccnc(N)c5c4)ccc32)CC1]

113. A compound of formula (IV), or a pharmaceutically acceptable salt thereof: 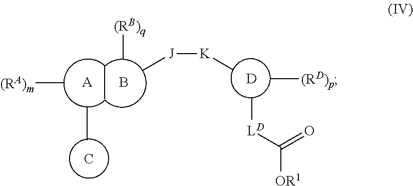
wherein:
ring 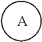
is arylene or heteroarylene;
ring 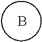
is arylene or heteroarylene;
ring 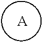
is fused to ring 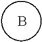
at two and only two adjacent positions;
ring 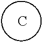
is selected from the group consisting of 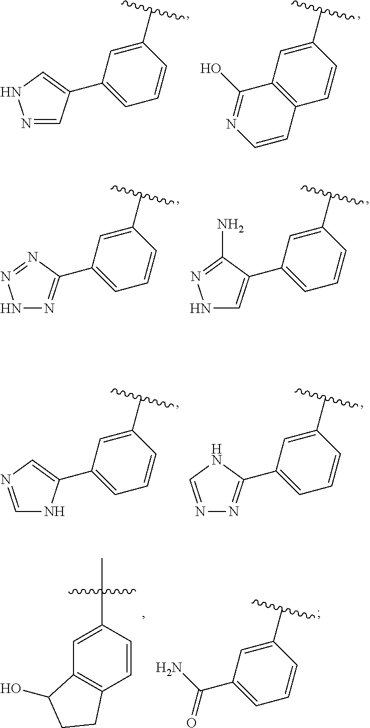
ring 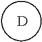
is aryl or heteroaryl;
J represents —CH₂—, —NH—, —CH₂CH₂—, —C(O)—, —C≡C—, —O—, —S—, —S(O)—, —SO₂—, —N(alkyl)-, —CH(alkyl)-, —CH(aryl)-, —C(alkyl)₂-, —CH(cycloalkyl)-, or 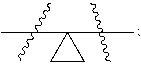 [SMILES: *C1(*)CC1]
K represents a bond, —O—, —NH—, —C(O)—, —CH₂—, —S—, —S(O)—, —SO₂—, —N(alkyl)-, —CH(alkyl)-, or —CH(cycloalkyl)-;
wherein at least one of J and K is a bond, —C(O)—, —CH₂—, —CH₂CH₂—, —CH(alkyl)-, or —CH(aryl)₂-;
L^{D} represents —CH₂—, —CH₂CH₂—, —CF₂—, —CH(F)—, —CD₂-, —CH(D)-, —CH(OH)—, —CH(alkyl)-, —CH(cycloalkyl)-, —CHNH₂—, —CH(NH(alkyl))-, —CH(NH(cycloalkyl))-, or a bond;
R^{A}, independently for each occurrence, represents H, halo, hydroxyl, cyano, amino, alkyl, optionally substituted alkoxy, hydroxyalkyl, optionally substituted aryloxy, (aryloxy)alkyl, (cycloalkyl)alkoxy, (heterocycloalkyl)alkoxy, optionally substituted (heteroaryl)alkoxy, haloalkyl, haloalkoxy, (hydroxy)haloalkyl, alkoxyalkyl, optionally substituted aminoalkyl, optionally substituted alkynyl, optionally substituted aryl, optionally substituted heteroaryl, optionally substituted heteroarylalkyl, optionally substituted cycloalkyl, optionally substituted (cycloalkyl)alkyl, optionally substituted (cycloalkyl)alkenyl, optionally substituted heterocycloalkyl, optionally substituted (heterocycloalkyl)alkyl, —C(O)OH, —C(O)NH₂—, —C(O)N(alkyl)₂-, —CH₂C(O)OH, —NO₂, —CH₂NH(optionally substituted alkyl), —CH₂NH(Boc), —CH₂N(Boc)(optionally substituted alkyl), —CH₂NH((cycloalkyl)alkyl), —CH₂N(alkyl)(cycloalkyl), —CH₂N(alkyl)((cycloalkyl)alkyl), —NH(optionally substituted alkyl), —NH(cycloalkyl), —NH((cycloalkyl)alkyl), —NH((heterocycloalkyl)alkyl), —N(alkyl)₂, —N(alkyl)((cycloalkyl)alkyl, —N(alkyl)((heterocycloalkyl)alkyl, —NH(heteroarylalkyl), —CHO(optionally substituted aryl), —C(O)O(alkyl), —C(O)NH(optionally substituted alkyl), —C(O)NH((cycloalkyl)alkyl), —NHC(O)O(alkyl), or —CH₂N(alkyl)₂;
R^{B}, independently for each occurrence, represents H, oxo, —C(O)O(alkyl), halogen, cyano, amino, —C(O)OH, —CH₂C(O)OH, —C(O)NH₂, —C(O)NH(cycloalkyl), —C(O)NH(alkyl), —C(O)NH(aryl), —C(O)NH(heteroaryl), —C(O)(alkyl), —S(O)₂alkyl, alkylaminoalkyl, alkylaminocycloalkyl, alkoxyalkyl, hydroxyalkyl, haloalkyl, (hydroxy)haloalkyl, or tosyl, or is optionally substituted alkyl, aryl, heteroaryl, cycloalkyl, spirocycloalkyl, halocycloalkyl, (cycloalkyl)alkyl, heterocycloalkyl, spiroheterocycloalkyl, or (heterocycloalkyl)alkyl; or two geminal occurrences of R^{B} taken together with the atom to which they are attached form an optionally substituted spirocycloalkyl or a spiroheterocycloalkyl;
R^{D}, independently for each occurrence, represents H, halo, hydroxyl, cyano, —NH₂, —NH(Ac), —NH(alkyl), —N(alkyl)₂, —NH(CO)(alkyl), —CH₂NH₂, —CH₂NHC(O)(alkyl), —C(O)NH₂, —C(O)OH, or —NHC(O)O(alkyl), or is optionally substituted alkyl, alkoxy, cycloalkyl, (cycloalkyl)alkyl, hydroxyalkyl, aminoalkyl, haloalkoxy, or haloalkyl;
R¹ represents H or optionally substituted alkyl; and
m, p, and q are each independently 0, 1, or 2.

114. The compound of claim 113, wherein:
ring 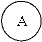
is arylene;
ring 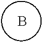
is heteroarylene;
ring 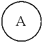
is fused to ring 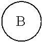
at two and only two adjacent positions;
ring 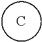
is selected from the group consisting of 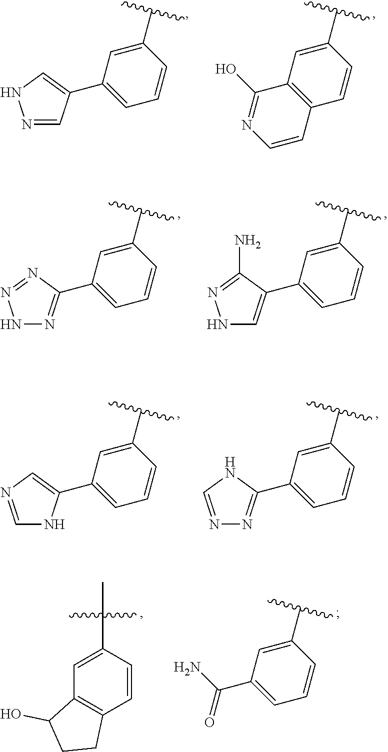
ring 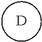
is aryl;
J represents —CH₂—;
K represents a bond or —O—;
LP represents —CH₂—;
R^{A} represents H;
R^{B}, independently for each occurrence, represents H, oxo, or alkyl;
R^{D} represents H;
R¹ represents H; and
m, p, and q are each independently 0, 1, or 2.

115. The compound of claim 113 or 114, having the structure of formula (IVa):

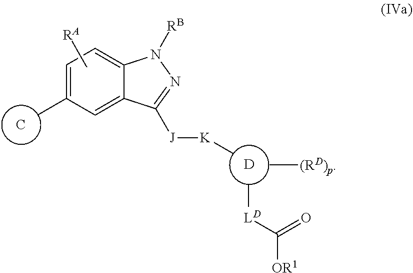

116. The compound of any one of claim 113 or 115, wherein J represents —CH₂—, —NH—, —CH₂CH₂—, —C(O)—, —O—, —S—, —S(O)—, —SO₂—, —N(alkyl)-, —CH(alkyl)-, —CH(aryl)-, —C(alkyl)₂-, —CH(cycloalkyl)-, or

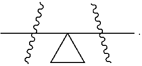 [SMILES: *C1(*)CC1]

117. The compound of any one of claims 113-116, wherein -J-K- represents —CH₂—O— or CH₂—.

118. The compound of any one of claims 113-117, wherein 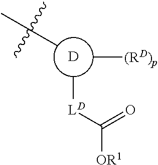
represents

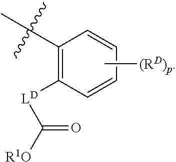

119. The compound of claim 113, wherein ring 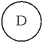
is bicyclic heteroaryl.

120. The compound of claim 119, wherein 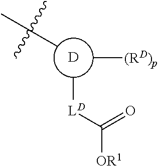
represents

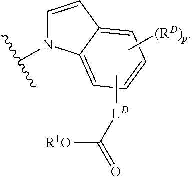

121. The compound of claim 119, wherein 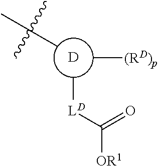
represents

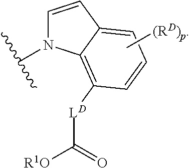

122. The compound of any one of claims 113-121, wherein R¹ is H.

123. The compound of any one of claims 113-122, wherein L^{D} represents —CH₂—.

124. The compound of any one of claims 113-123, wherein R^{D} represents H.

125. The compound of any one of claims 113-124, wherein R^{B}, independently for each occurrence, represents H, oxo, or alkyl.

126. The compound of any one of claims 113-125, wherein R^{A} represents H.

127. The compound of claim 113, selected from the following table:

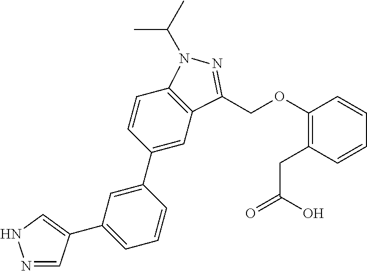 [SMILES: CC(C)n1nc(COc2ccccc2CC(=O)O)c2cc(-c3cccc(-c4cn[nH]c4)c3)ccc21]

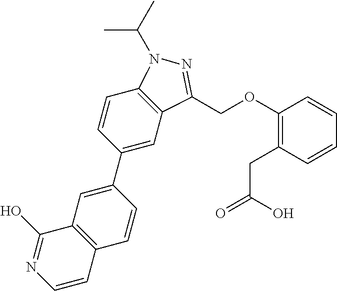 [SMILES: CC(C)n1nc(COc2ccccc2CC(=O)O)c2cc(-c3ccc4ccnc(O)c4c3)ccc21]

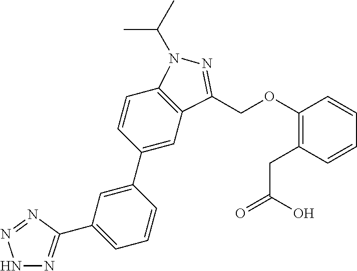 [SMILES: CC(C)n1nc(COc2ccccc2CC(=O)O)c2cc(-c3cccc(-c4nn[nH]n4)c3)ccc21]

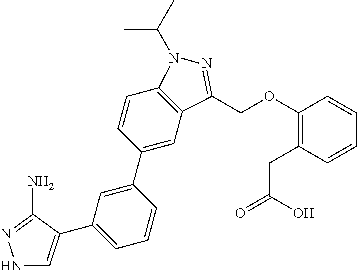 [SMILES: CC(C)n1nc(COc2ccccc2CC(=O)O)c2cc(-c3cccc(-c4c[nH]nc4N)c3)ccc21]

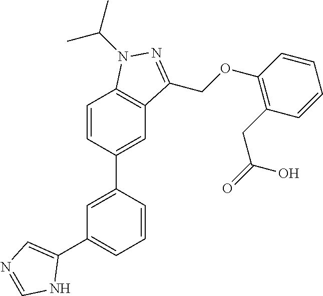 [SMILES: CC(C)n1nc(COc2ccccc2CC(=O)O)c2cc(-c3cccc(-c4cnc[nH]4)c3)ccc21]

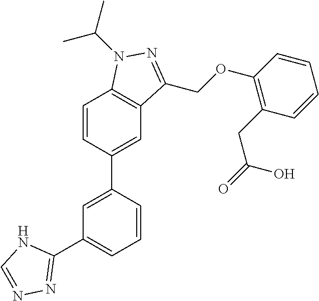 [SMILES: CC(C)n1nc(COc2ccccc2CC(=O)O)c2cc(-c3cccc(-c4nnc[nH]4)c3)ccc21]

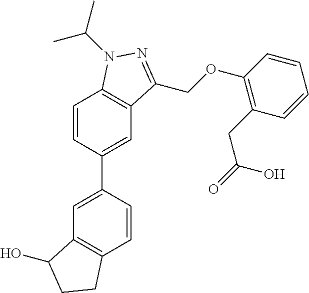 [SMILES: CC(C)n1nc(COc2ccccc2CC(=O)O)c2cc(-c3ccc4c(c3)C(O)CC4)ccc21]

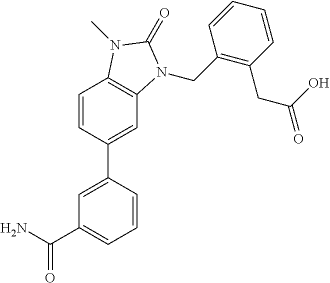 [SMILES: Cn1c(=O)n(Cc2ccccc2CC(=O)O)c2cc(-c3cccc(C(N)=O)c3)ccc21]

128. A pharmaceutical composition, comprising a compound of any one of claims 1-127, or a pharmaceutically acceptable salt thereof; and a pharmaceutically acceptable carrier.

129. A method of treating or preventing a disease or condition characterized by aberrant kallikrein activity, comprising administering to a subject in need thereof a therapeutically effective amount of a compound of any one of claims 1-127, or a pharmaceutically acceptable salt thereof.

130. The method of claim 129, wherein the disease or condition is characterized by aberrant kallikrein-related peptidase 5 (KLK5) activity.

131. The method of claim 129 or 130, wherein the disease or condition is a skin disease.

132. The method of any one of claims 129-131, wherein the disease or condition is Netherton Syndrome.

133. The method of claim 131, wherein the skin disease is eczema, atopic eczema, atopic dermatitis, ichthyosiform (scaly) erythroderma (IE), rosacea, or a skin infection.

134. The method of claim 129 or 130, wherein the disease or condition is selected from the group consisting of hypersensitivity of the immune system (atopy), hyper IgE syndrome, allergies (including allergies to food and airborne agents), asthma, allergic asthma, chronic inflammation, rhinitis, conjunctivitis, angioedema, cosinophilia, eosinophilic esophagitis, growth delay, failure to thrive, trichorrhexis invaginata (TI), respiratory tract infections, systemic infections, and gastrointestinal disorders.

135. The method of claim 129 or 130, wherein the disease or condition is cancer.

136. The method of claim 135, wherein the cancer is selected from the group consisting of ovarian cancer, breast cancer, prostate cancer, bladder cancer, cervical cancer, glioblastoma multiforme, and neuroblastoma.

137. A method of treating or preventing a disease or condition characterized by aberrant kallikrein activity, comprising administering to a subject in need thereof a therapeutically effective amount of a compound, or a pharmaceutically acceptable salt thereof; wherein the compound is selected from the group consisting of:

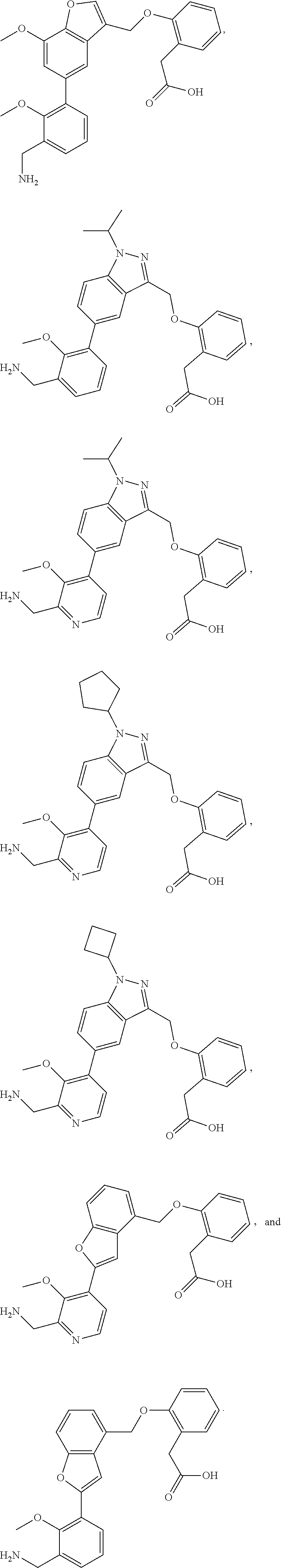

138. The method of claim 137, wherein the disease or condition is characterized by aberrant kallikrein-related peptidase 5 (KLK5) activity.

139. The method of claim 137 or 138, wherein the disease or condition is a skin disease.

140. The method of any one of claims 137-139, wherein the disease or condition is Netherton Syndrome.

141. The method of claim 139, wherein the skin disease is eczema, atopic eczema, atopic dermatitis, ichthyosiform (scaly) erythroderma (IE), rosacea, or a skin infection.

142. The method of claim 137 or 138, wherein the disease or condition is selected from the group consisting of rhinitis, conjunctivitis, angioedema, eosinophilia, eosinophilic esophagitis, growth delay, failure to thrive, trichorrhexis invaginata (TI), respiratory tract infections, systemic infections and gastrointestinal disorders.

143. The method of claim 137 or 138, wherein the disease or condition is selected from the group consisting of hypersensitivity of the immune system (atopy), hyper IgE syndrome, allergies (including allergies to food and airborne agents), asthma, allergic asthma, and chronic inflammation.

144. The method of claim 137 or 138, wherein the disease or condition is cancer.

145. The method of claim 144, wherein the cancer is selected from the group consisting of ovarian cancer, breast cancer, prostate cancer, bladder cancer, cervical cancer, glioblastoma multiforme, and neuroblastoma.
